(19) United States
(12) Patent Application Publication
(10) Pub. No.: US 2024/0294499 A1
(43) Pub. Date: Sep. 5, 2024

(54) TARGETED PROTEIN DEGRADATION USING BIFUNCTIONAL COMPOUNDS THAT BIND UBIQUITIN LIGASE AND TARGET MCL-1 PROTEIN

(71) Applicant: Captor Therapeutics S.A., Wroclaw (PL)

(72) Inventors: Sylvain COTTENS, Witterswil (CH); Magda DREWNIAK-SWITALSKA, Wroclaw (PL); Katarzyna KACZANOWSKA, Wroclaw (PL); Tomasz TOMCZYK, Kamieniec Wroclawski (PL); Andrzej TRACZ, Wilczyce (PL); Michal WALCZAK, Wroclaw (PL); Karolina WOJCIK, Wroclaw (PL)

(21) Appl. No.: 18/565,630

(22) PCT Filed: May 27, 2022
(86) PCT No.: PCT/EP2022/064481

§ 371 Date: Nov. 30, 2023

(30) Foreign Application Priority Data
Jun. 1, 2021 (WO) PCT/PL2021/000030

(51) Int. Cl. C07D 401/14; A61K 45/06; A61K 47/55; A61P 35/00; A61P 35/02; C07D 401/04; C07D 413/14; C07D 471/08; C07D 471/10; C07D 487/04; C07D 487/08; C07D 487/10; C07D 495/04; C07D 497/22; C07D 498/04

(52) U.S. Cl. CPC C07D 401/14; A61K 45/06; A61K 47/55; A61P 35/00; A61P 35/02; C07D 401/04; C07D 413/14; C07D 471/08; C07D 471/10; C07D 487/04; C07D 487/08; C07D 487/10; C07D 495/04; C07D 497/22; C07D 498/04

(57) ABSTRACT

A compound of formula (I); [MCL-1 ligand moiety]-[linker]-[ligase ligand moiety] (I); or a salt, solvate, hydrate, isomer or prodrug thereof, wherein [MCL-1 ligand moiety] is a compound of Formula (A), Formula (B) or Formula (C), and its use in the treatment of cancer.

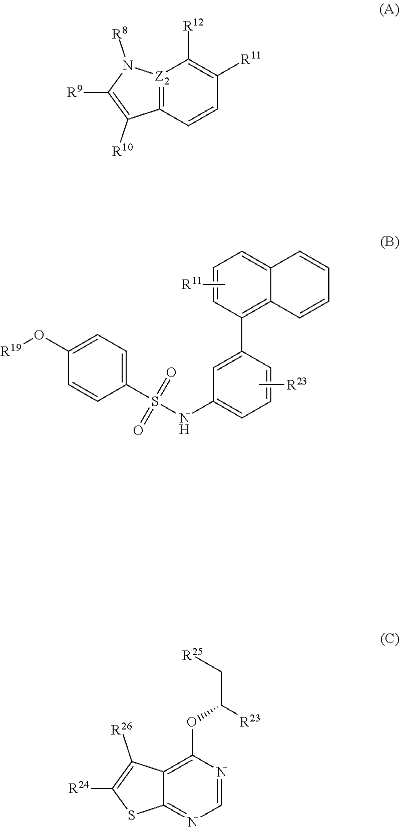

### FIELD OF THE INVENTION

The present invention relates to bifunctional compounds which can bind to a ubiquitin ligase and also to a target protein, such that the target protein is placed in proximity to the ubiquitin ligase in order to induce its degradation.

### BACKGROUND

The Ubiquitin-Proteasome System (UPS) is responsible for the maintenance of healthy and well-balanced proteome. In the process of ubiquitination, ubiquitin units are covalently attached to the protein, forming a polyubiquitin chain, which marks the protein for degradation via the proteasome. Ubiquitination is central to the regulation of nearly all cellular processes and is also tightly regulated itself. Ubiquitin ligases facilitate ubiquitination of different proteins in vivo and contribute to precise regulation of the system. Upon recognition, the ubiquitin ligases mediate the attachment of ubiquitin moieties to the target protein, which label it for degradation by the proteasome.

The idea of selective target protein degradation (TPD) by modulation of UPS was first described in 1999 (US2002173049 A1 (PROTEINIX INC) 21 Nov. 2002). One approach to TPD is by the use of bifunctional molecules that bind the ubiquitin ligase and the target protein simultaneously, allowing for efficient ubiquitin transfer to the latter. This concept was first described by Sakamoto K M et al. (Proc Natl Acad Sci USA. 2001 Jul. 17; 98(15):8554-9) and more recently reviewed by Burslem G M and Crews C M (Cell. 2020 Apr. 2; 181(1):102-114).

Oncogenic stress, such as DNA damage, may result in programmed cell death, the cellular response meant to prevent the oncogenic transformation. This mechanism depends on an interplay between pro-apoptotic and anti-apoptotic Bcl-2 proteins, and the balance of these proteins is essential for the proper functioning of the cell.

BCL-2, BCL-xL and MCL-1 are BH3-domain-containing anti-apoptotic proteins. These proteins bind to effector Bcl-2 proteins Bak and Bax (via their BH3 domains), preventing their pro-apoptotic activity. Inhibition of BH3 domain—BH3 pocket binding interface is a well-known approach to cancer therapy (Leber B, Kale J, Andrews D W. Cancer Discov. 2018 December; 8(12):1511-1514).

High expression of induced myeloid leukaemia cell differentiation protein (MCL-1) is observed in many human cancers and is associated with resistance to cytotoxic drugs. Research shows that inhibition of MCL-1 protein in some malignancies leads to the release of pro-apoptotic proteins and induction of apoptosis. Therefore, targeting MCL-1 can be applied as a therapeutic strategy in these types of cancer which are MCL-1 dependent, such as multiple myeloma, acute myeloid leukaemia, chronic myeloid leukaemia, B-cell acute lymphoblastic leukaemia, hepatocellular carcinoma and non-small cell lung cancers. This concept was confirmed in vitro and in vivo (Tron A E et al. Nat Commun. 2018 Dec. 17; 9(1):5341). Also, treatment with Bcl-2 inhibitors and MEK inhibitors often elicits MCL-1 dependence and subsequent inactivation of MCL-1 results in synthetic lethality (Leber B, Kale J, Andrews D W. Cancer Discov. 2018 December; 8(12):1511-1514). As demonstrated by Montero, J. et al. (Nat. Commun. 10, 5157 (2019)) and Sale, M. J. et al. (Nat. Commun. 10, 5167 (2019)), MCL-1 is a driver of adaptive survival in tumor cells treated with oncogene targeted therapies, therefore MCL-1 targeting drugs are likely to overcome cancer resistance to these therapeutics.

In parallel to the efforts focused on inhibition of MCL-1, targeted degradation appears as an attractive therapeutic alternative. Both Papatzimas et al. (J. Med. Chem. 2019, 62, 11, 5522-5540) and Wang Z et al. (J. Med. Chem. 2019, 62, 17, 8152-8163) have demonstrated degradation of the MCL-1 protein. However, the potency of reported compounds in terms of cellular degradation and the ability to induce apoptosis remains suboptimal. Therefore, alternative chemotypes with improved potency are needed to develop therapeutically applicable MCL-1 degraders.

One of the challenges in the development of MCL-1 targeted therapeutics is related to safety, as MCL-1 has been shown to be essential for cardiac homeostasis in adult murine models, and the absence of MCL-1 led to loss of cardiomyocytes. Clinical trials involving MCL-1 inhibitors are currently on clinical hold to evaluate a safety signal for cardiac toxicity (Wei A H et al. Blood Rev. 2020 November; 44: 100672).

### SUMMARY OF INVENTION

In accordance with a first aspect of the invention, there is provided a compound of formula (I)

[MCL-1 ligand moiety]-linker-[ligase ligand moiety] (I)

or a salt, solvate, hydrate, isomer or prodrug thereof,
wherein [ligase ligand moiety] is:

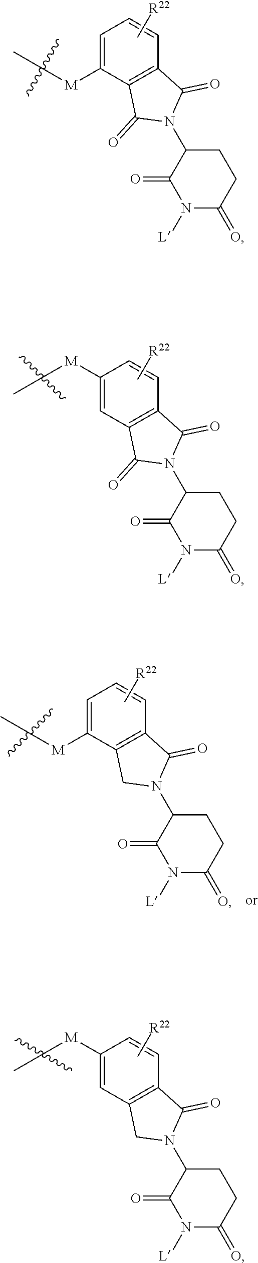

wherein
M is O, S or NH, or is absent;
[symbol] indicates attachment to R¹⁸ of the linker;
R²² is hydrogen, halogen or an amino group; and
L′ is hydrogen, alkyl, benzyl, acetyl or pivaloyl;
   [MCL-1 ligand moiety] is a compound of Formula (A), Formula (B) or Formula (C)

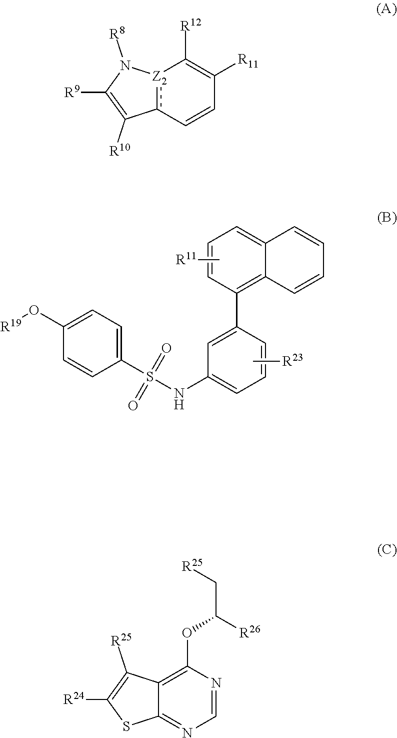

wherein
[symbol] is a single bond or a double bond;
R⁸ is H, R¹⁹, or C₁-C₆ alkyl optionally substituted with morpholine;
R⁹ is —C(O)OH, —C(O)OC₁-C₆alkyl; —C(O)NH₂; —C(O)OR¹⁹ or —C(O)NHR¹⁹,
R¹⁰ is —C₂₋₅alkyl-O—R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹¹ is H, halogen or C₁-C₆ alkyl,
R¹² is H,

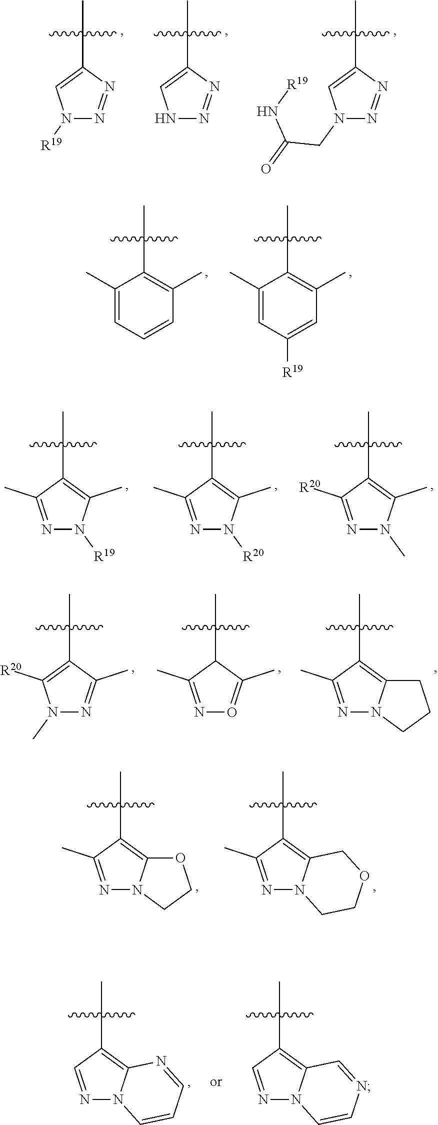

wherein R²⁰ s Me, —CH₂—OMe, —CH₂—O-bromobenzaldehyde, or

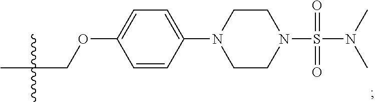 [SMILES: *COc1ccc(N2CCN(S(=O)(=O)N(C)C)CC2)cc1]

or when R¹² is

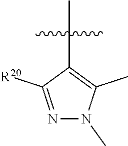

and R¹⁰ is —O-naphthyl substituted with —O— or —S—, then R²⁰ is

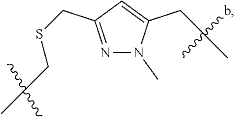

wherein [symbol] indicates attachment to —O— or —S— of R¹⁰;
and wherein
R¹⁹ is a bond connected to R¹⁴ of the linker;
R²³ is —C(O)OH or —C(O)OC₁-C₆alkyl;
Z₂ is N or C, wherein when Z₂ is N, then [symbol] is a single bond; and when Z₂ is C, then [symbol] is a double bond,
R²⁴ is furan optionally substituted with at least one halogen,
each R²⁵ is independently phenyl substituted with —OR²⁸ and optionally further substituted with at least one substituent selected from halogen and C₁-C₆ alkyl;
R²⁶ is —C(O)OR¹⁹ or —C(O)NHR¹⁹; and
each R²⁸ is independently —C₁₋₃alkyl-(N-alkyl piperazine) or —C₁₋₃alkyl-(N-haloalkylpyrazole)
and wherein each of Formula (A), Formula (B) and Formula (C) contains a single R¹⁹;
   and wherein [linker] has the following formula

R¹⁴—R¹⁵—R¹⁶—R¹⁷—R¹⁸

wherein
R¹⁴ is —C₁₋₆alkyl, —C₂₋₆alkenyl, —C₂₋₆alkynyl, C₁₋₆ alkyl-N(C₁₋₆ alkyl)-, —C(O)—, —SO₂— or is absent
R¹⁵ is cycloalkyl, heterocycloalkyl, aryl, heteroaryl, —C₁₋₆ alkyl-NH—, C₁₋₆ alkyl-N(C₁₋₆ alkyl)-, -cycloalkyl-NH—, -heterocycloalkyl-NH—, or is absent
R¹⁶ is —C₁₋₆alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, (C₂H₄—O)ₓ, (C₃H₆—O)ₓ, or is absent
x is 1-10
y is 2-10
R¹⁸ is —C₁₋₆ alkyl, heterocycloalkyl, or is absent
wherein at least one of R¹⁴-R¹⁸ is present
   with the proviso that:
   when
R¹⁰ is —C₃H₆—O-naphthyl,
R¹² is

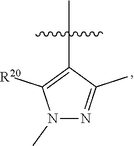

and
R²⁰ is

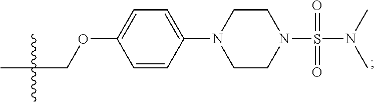 [SMILES: *COc1ccc(N2CCN(S(=O)(=O)N(C)C)CC2)cc1]

then R⁹ is —C(O)OH, —C(O)OC₁-C₆alkyl or —C(O)NH₂, and [ligase ligand moiety] is

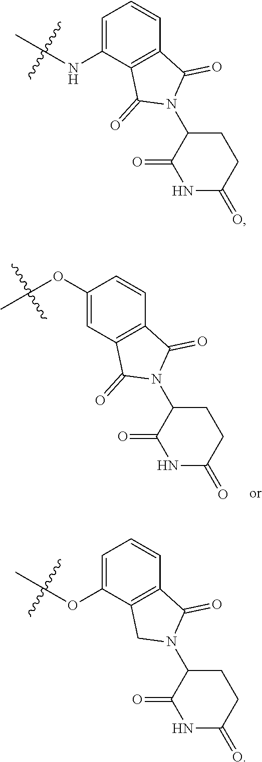 [SMILES: *Nc1cccc2c1C(=O)N(C1CCC(=O)NC1=O)C2=O ; *Oc1ccc2c(c1)C(=O)N(C1CCC(=O)NC1=O)C2=O ; "or" ; *Oc1cccc2c1CN(C1CCC(=O)NC1=O)C2=O]

In some embodiments, R²² is hydrogen or an amino group. In some embodiments, R²² is hydrogen.

In some embodiments, L′ is hydrogen or methyl. In some embodiments, L′ is hydrogen.

In some embodiments, M is O or NH, or is absent.

In some embodiments, [ligase ligand moiety] is:

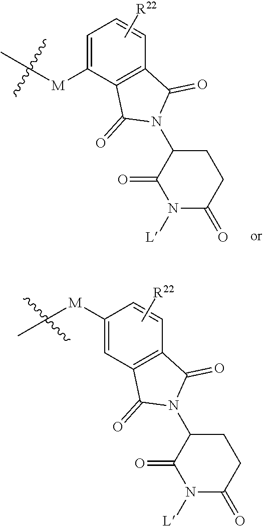

In some embodiments, [ligase ligand moiety] is:

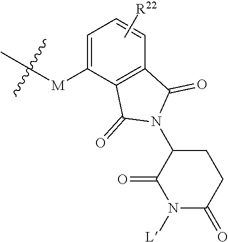

In some embodiments, [ligase ligand moiety] is:

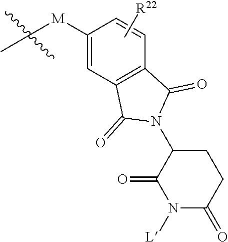

In some embodiments, [ligase ligand moiety] is

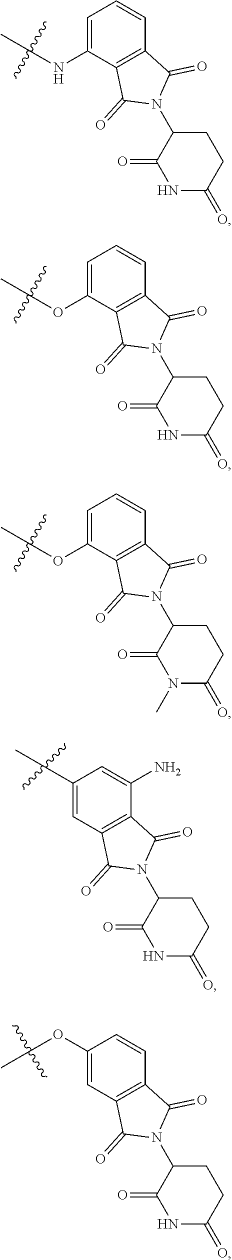 [SMILES: *Nc1cccc2c1C(=O)N(C1CCC(=O)NC1=O)C2=O ; *Oc1cccc2c1C(=O)N(C1CCC(=O)NC1=O)C2=O ; *Oc1cccc2c1C(=O)N(C1CCC(=O)N(C)C1=O)C2=O ; *c1cc(N)c2c(c1)C(=O)N(C1CCC(=O)NC1=O)C2=O ; *Oc1ccc2c(c1)C(=O)N(C1CCC(=O)NC1=O)C2=O]

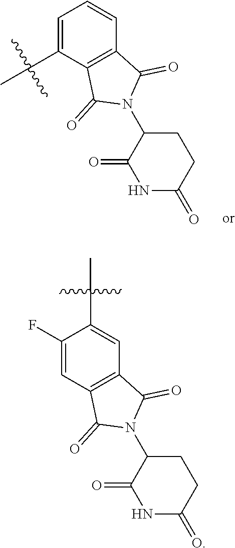 [SMILES: *c1cccc2c1C(=O)N(C1CCC(=O)NC1=O)C2=O ; "or" ; *c1cc2c(cc1F)C(=O)N(C1CCC(=O)NC1=O)C2=O]

In some embodiments, [ligase ligand moiety] is

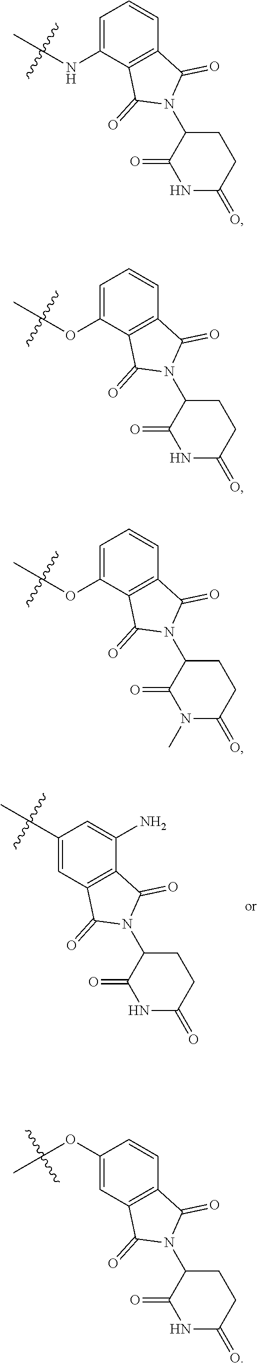 [SMILES: *Nc1cccc2c1C(=O)N(C1CCC(=O)NC1=O)C2=O ; *Oc1cccc2c1C(=O)N(C1CCC(=O)NC1=O)C2=O ; *Oc1cccc2c1C(=O)N(C1CCC(=O)N(C)C1=O)C2=O ; *c1cc(N)c2c(c1)C(=O)N(C1CCC(=O)NC1=O)C2=O ; *Oc1ccc2c(c1)C(=O)N(C1CCC(=O)NC1=O)C2=O]

In some embodiments, [ligase ligand moiety] is:

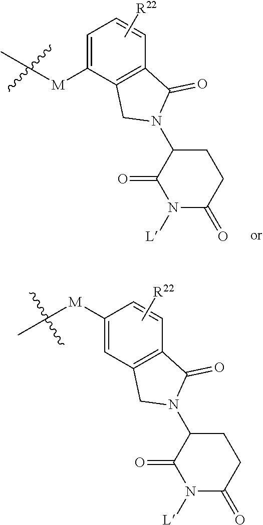

In some embodiments, [ligase ligand moiety] is:

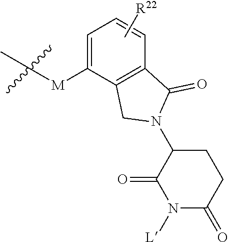

In some embodiments, [ligase ligand moiety] is:

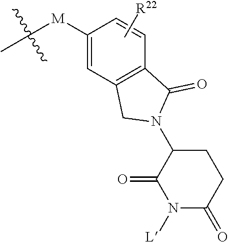

In some embodiments, [ligase ligand moiety] is

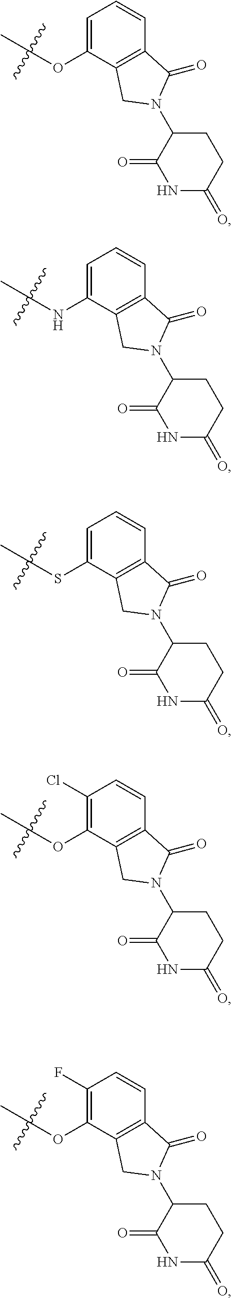 [SMILES: *Oc1cccc2c1CN(C1CCC(=O)NC1=O)C2=O ; *Nc1cccc2c1CN(C1CCC(=O)NC1=O)C2=O ; *Sc1cccc2c1CN(C1CCC(=O)NC1=O)C2=O ; *Oc1c(Cl)ccc2c1CN(C1CCC(=O)NC1=O)C2=O ; *Oc1c(F)ccc2c1CN(C1CCC(=O)NC1=O)C2=O]

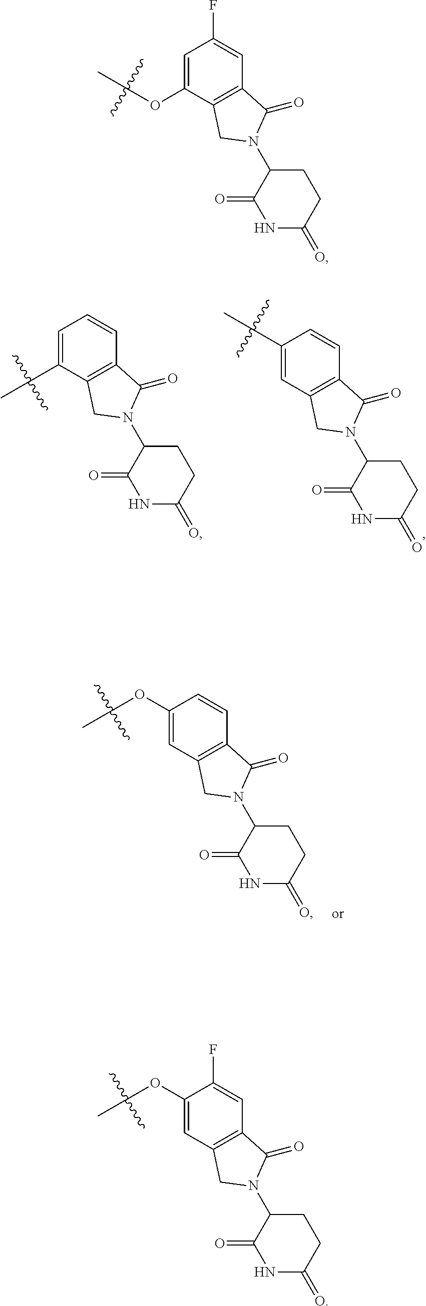 [SMILES: *Oc1cc(F)cc2c1CN(C1CCC(=O)NC1=O)C2=O ; *c1cccc2c1CN(C1CCC(=O)NC1=O)C2=O ; *c1ccc2c(c1)CN(C1CCC(=O)NC1=O)C2=O ; *Oc1ccc2c(c1)CN(C1CCC(=O)NC1=O)C2=O ; *Oc1cc2c(cc1F)C(=O)N(C1CCC(=O)NC1=O)C2]

In some embodiments, [ligase ligand moiety] is

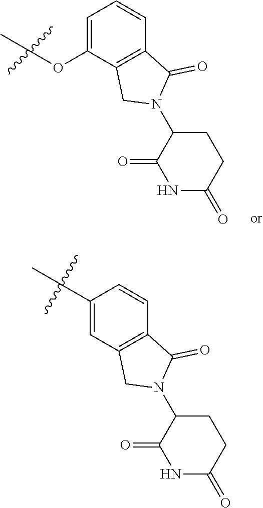 [SMILES: *Oc1cccc2c1CN(C1CCC(=O)NC1=O)C2=O ; "or" ; *c1ccc2c(c1)CN(C1CCC(=O)NC1=O)C2=O]

In some embodiments, R¹⁴ is —C₁₋₆ alkyl, —C₂₋₆ alkenyl, —C₂₋₆ alkynyl, —C(O)—, —SO₂— or is absent.

In some embodiments, R¹⁵ is cycloalkyl, heterocycloalkyl, aryl, heteroaryl, —C₁₋₆ alkyl-NH—, -cycloalkyl-NH— or is absent.

In some embodiments,
R¹⁴ is —C₁₋₆ alkyl, —C₁₋₆ alkyl-N(Me)-, —SO₂— or is absent;
R¹⁵ is piperazine, bridged piperazine, piperazine N-oxide, piperazine cation, —C₁₋₆ alkyl-NH—, —C₁₋₆ alkyl-N(Me)-,

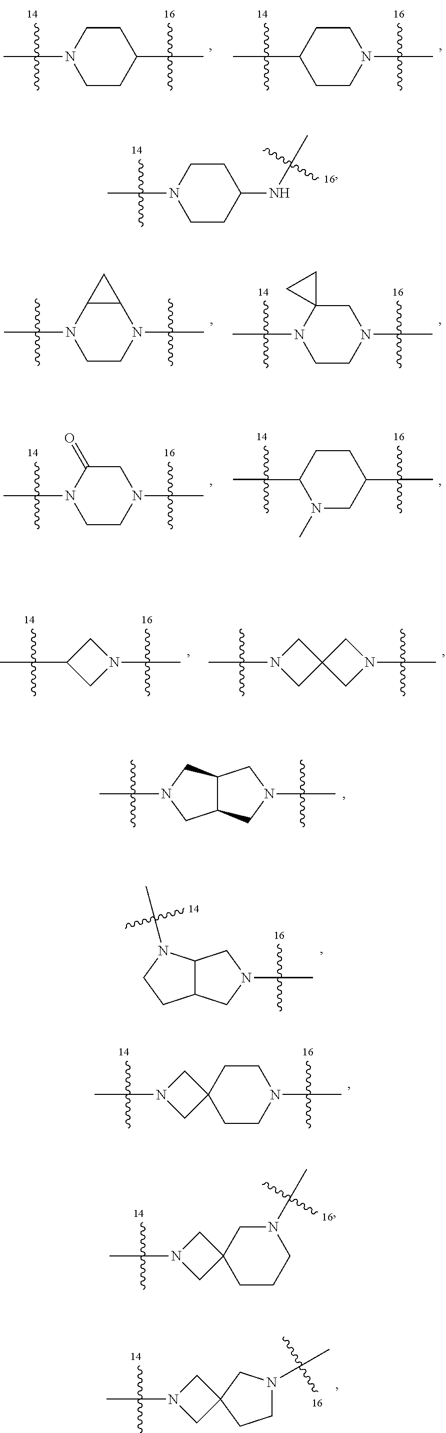

or is absent, wherein [symbol] indicates attachment to R¹⁴ and [symbol] indicates attachment to R¹⁶,
R¹⁷ is —CH₂(C₂H₄—O)_{y}, (C₂H₄—O)ₓ, (C₃H₆—O)ₓ, or is absent, wherein x is 1-6 and y is 2-6; and
R¹⁸ is —C₁₋₆ alkyl, piperazine,

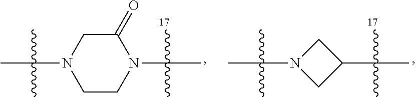 [SMILES: *N1CCN(*)C(=O)C1 ; *C1CN(*)C1]

or is absent, wherein
[symbol] indicates attachment to R¹⁷,
and wherein at least one of R¹⁴-R¹⁸ is present.

In some embodiments,
R¹⁴ is —C₁₋₆ alkyl, —SO₂— or is absent
R¹⁵ is piperazine, bridged piperazine, piperazine N-oxide, piperazine cation, —C₁₋₆ alkyl-NH—,

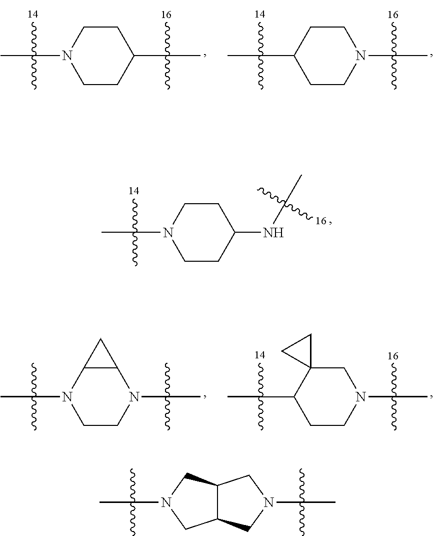 [SMILES: *C1CCN(*)CC1 ; *C1CCN(*)CC1 ; *NC1CCN(*)CC1 ; *N1CCN(*)C2CC21 ; *C1CCN(*)CC12CC2 ; *N1C[C@H]2CN(*)C[C@H]2C1]

or is absent, wherein [symbol] indicates attachment to R¹⁴ and [symbol] indicates attachment to R¹⁶,
R¹⁶ is —C₁₋₆ alkyl, —C(O)—, —C(O)—NH—, —CH₂—C(O)—NH— or is absent
R¹⁷ is —CH₂(C₂H₄—O)_{y}, (C₂H₄—O)ₓ, (C₃H₆—O)ₓ, or is absent
x is 1-6
y is 2-6
R¹⁸ is —C₁₋₆ alkyl, piperazine, or is absent
   wherein at least one of R¹⁴-R¹⁸ is present.

In some embodiments, R¹⁸ is —C₁₋₆ alkyl or is absent.

In some embodiments, when R¹⁴ is —SO₂—, at least two of R¹⁵-R¹⁸ are present, and at least one of R¹⁵-R¹⁸ is not C₁₋₆ alkyl.

In some embodiments, R¹⁴ is —SO₂—; R¹⁵ is —C₁₋₆ alkyl-NH—; R¹⁶ is —C(O)—; R¹⁷ is —CH₂(C₂H₄—O)_{y}, (C₂H₄—O)ₓ or is absent; and R¹⁸ is —C₂₋₄ alkyl. In some such embodiments, R¹⁵ is —C₂alkyl-NH—; x is 1 or 2; and y is 1.

In some embodiments, when R¹⁵ is piperazine, bridged piperazine, piperazine N-oxide, piperazine cation, —C₁₋₆ alkyl-NH—,

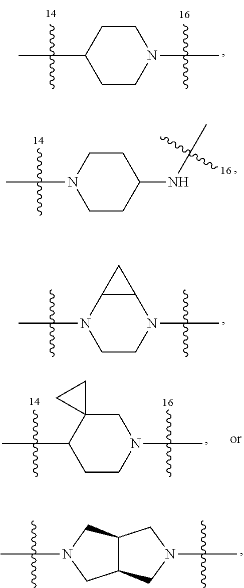

then R¹⁴ is —C₁₋₆ alkyl.

In some embodiments,
R¹⁴ is —C₁₋₆ alkyl,
R¹⁵ is piperazine, bridged piperazine, piperazine N-oxide,

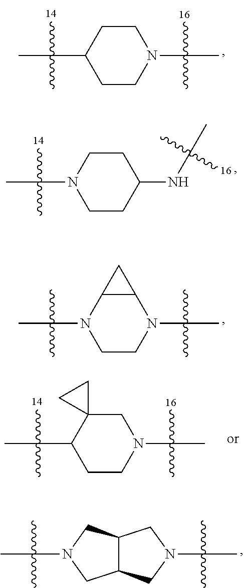

R¹⁶ is —C(O)—, —CH₂—C(O)—NH—, or is absent
R¹⁷ is —CH₂(C₂H₄—O)_{y}, (C₂H₄—O)ₓ, (C₃H₆—O)ₓ, or is absent, and
R¹⁸ is —C₁₋₆ alkyl.
   wherein when R¹⁶ and R¹⁷ are absent, R¹⁸ is —C₃₋₆ alkyl.

In some such embodiments, R¹⁴ is —C₂ alkyl; x is 1, 2 or 6; and y is 2.

In some embodiments, R¹⁴ is absent, R¹⁵ is absent, R¹⁶ is —C(O)—NH— or is absent; R¹⁷ is —CH₂(C₂H₄—O)_{y}, (C₂H₄—O)ₓ, (C₃H₆—O)ₓ, or is absent; and R¹⁸ is —C₁₋₆ alkyl.

In some embodiments, at least one of R¹⁴-R¹⁸ is not —C₁₋₆ alkyl.

In some embodiments, x is 1, 2 or 3; y is 2; and R¹⁸ is —C₂₋₆ alkyl.

In some embodiments, when R¹⁵ is —C₁₋₆ alkyl-NH—, at least one of R¹⁶—R¹⁸ is present.

In some embodiments, when R¹⁷ is —CH₂(C₂H₄—O)_{y}, (C₂H₄—O)ₓ or (C₃H₆—O)ₓ, at least one of R¹⁴—R¹⁶ and R¹⁸ is present, wherein at least one of R¹⁴ and R¹⁸ is not —C₁₋₆ alkyl.

In some embodiments, [linker] is selected from

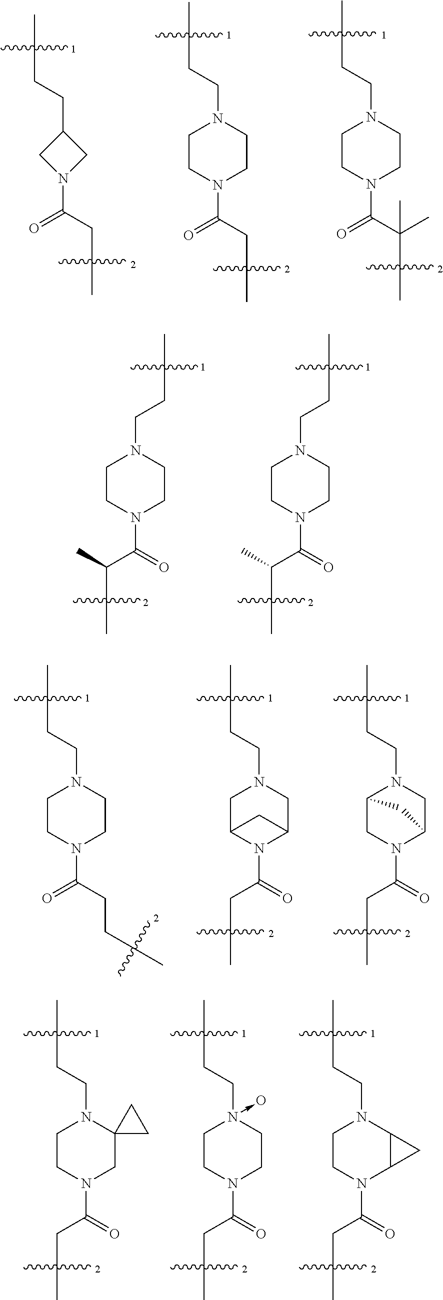

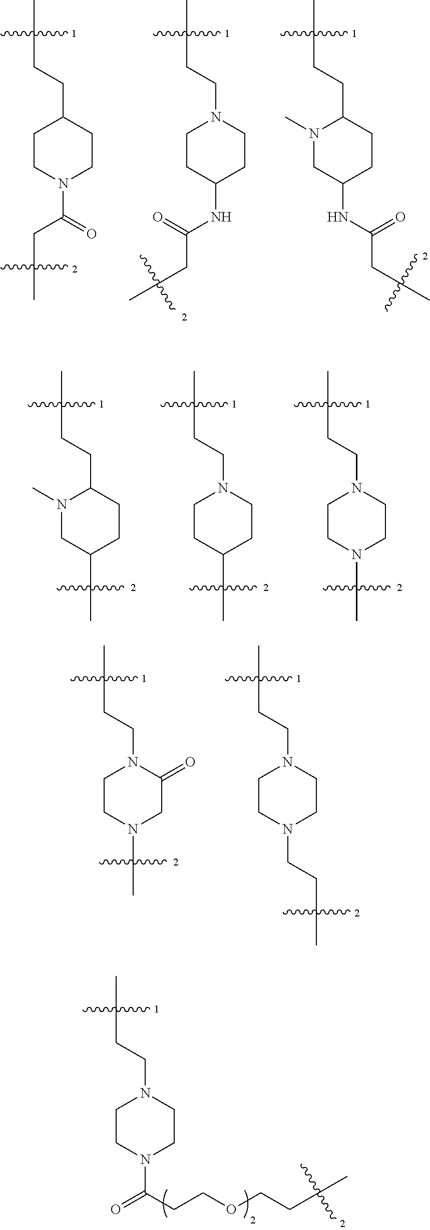

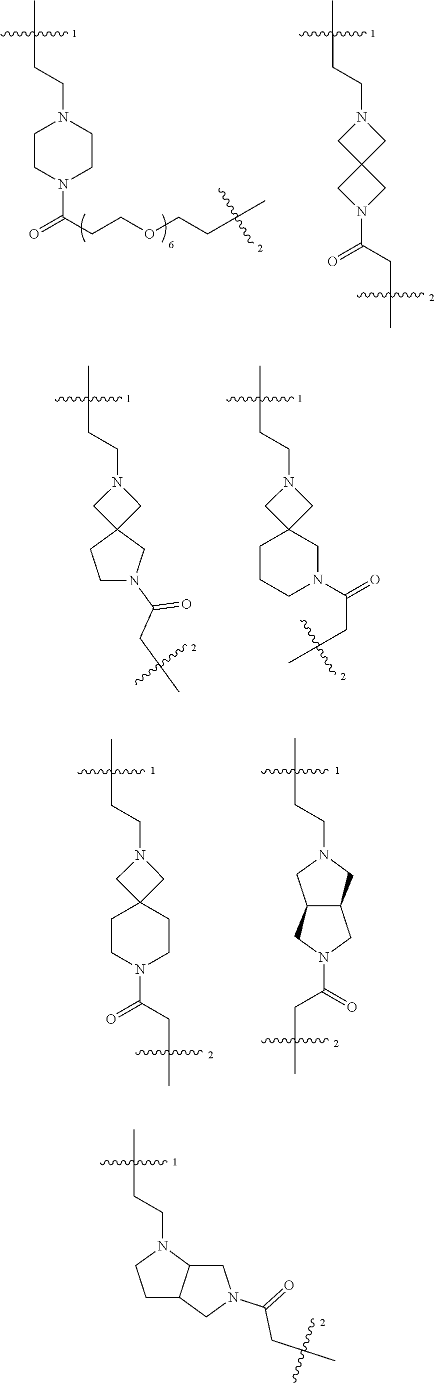

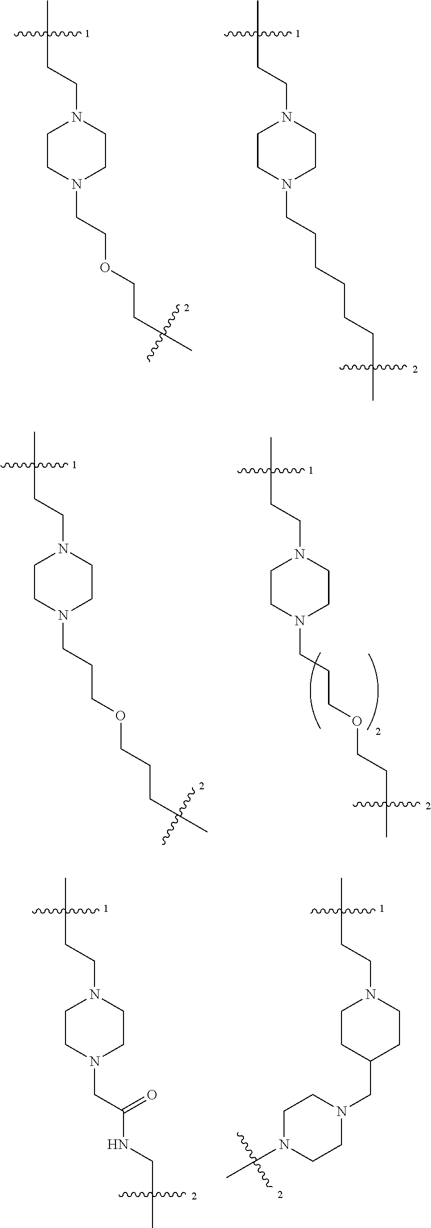

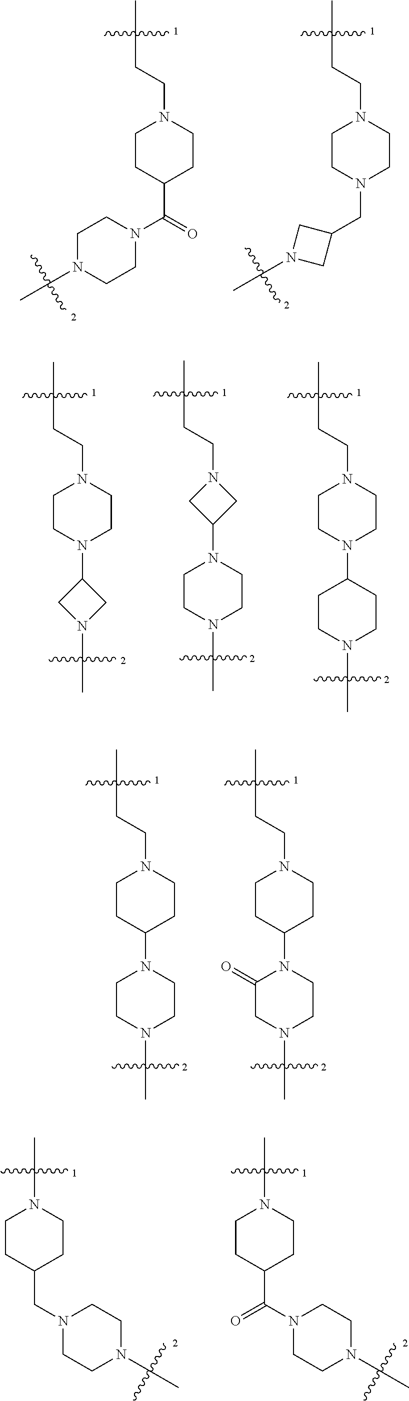

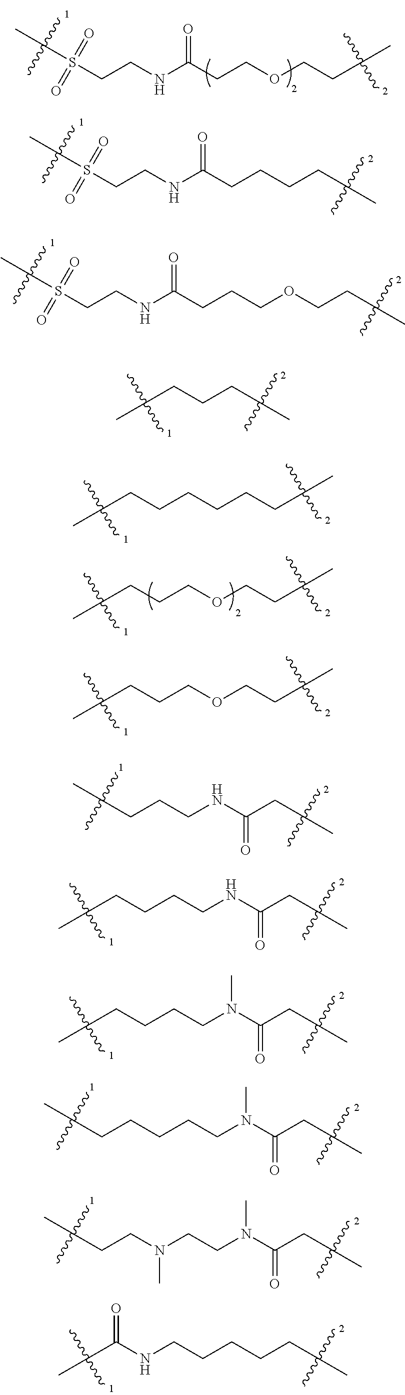

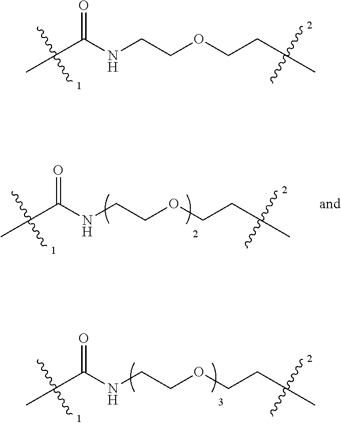

wherein
[symbol] indicates attachment to [MCL-1 ligand moiety] and
[symbol] indicates attachment to [ligase ligand moiety].

In some embodiments, [linker] is selected from

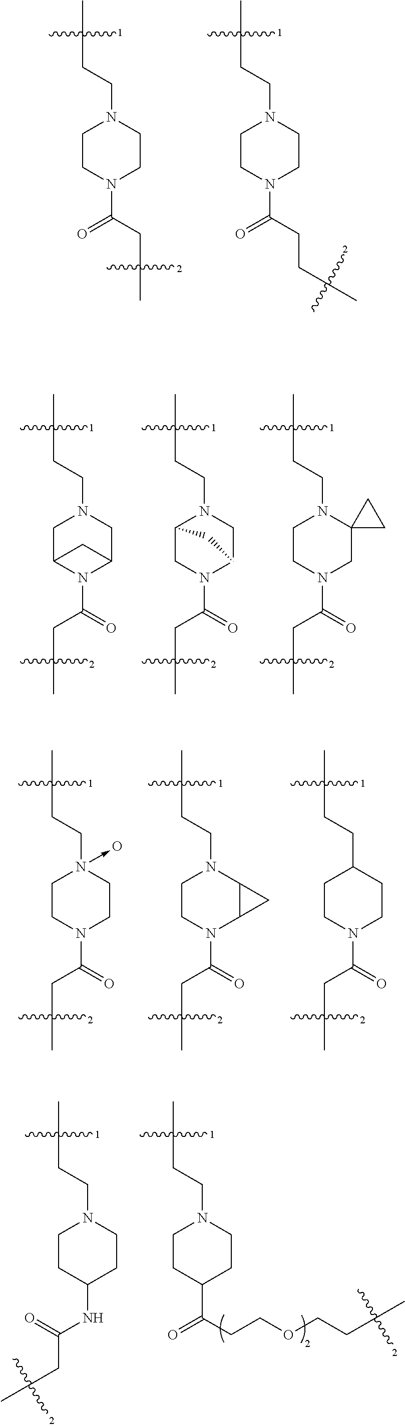

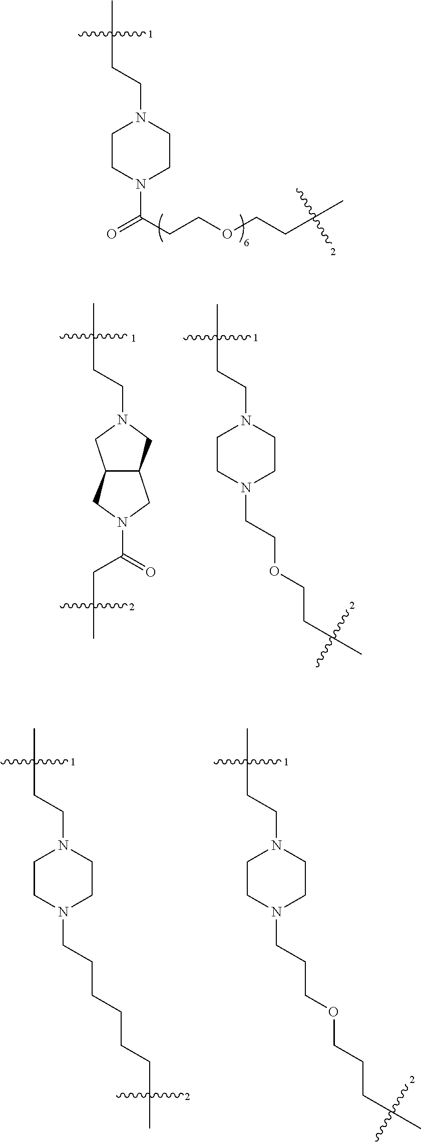

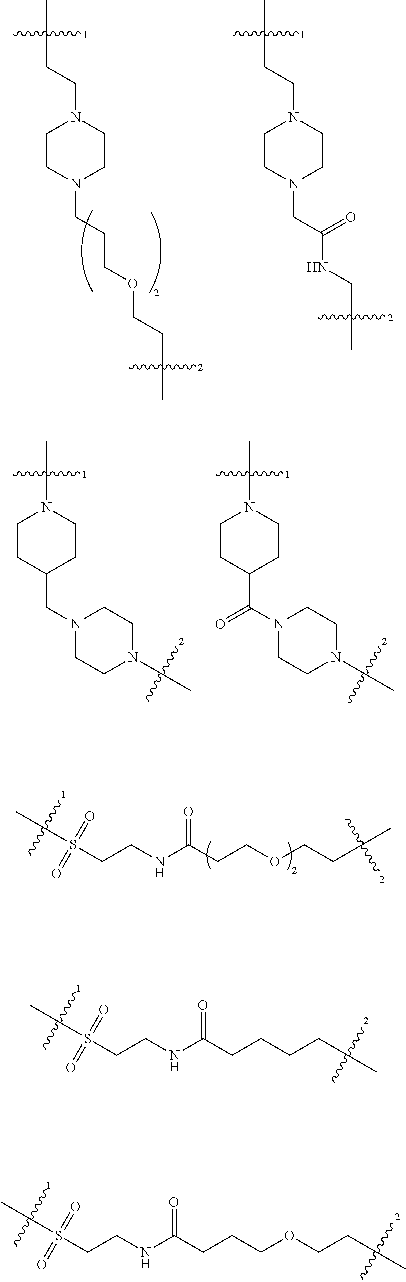

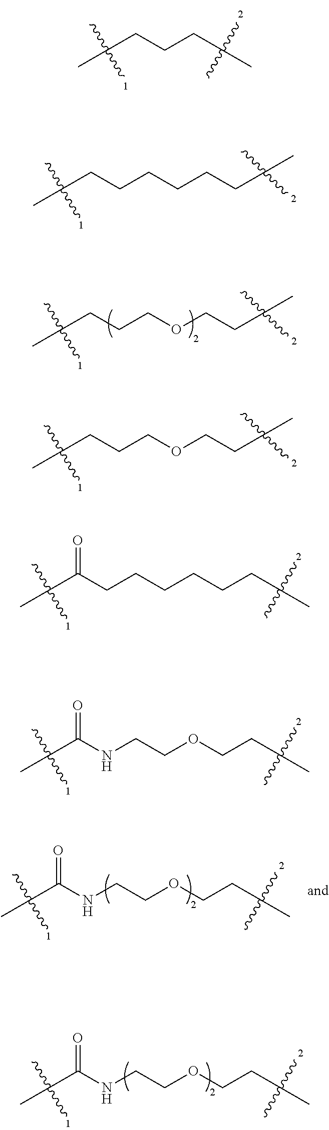

wherein
[symbol] indicates attachment to [MCL-1 ligand moiety] and
[symbol] indicates attachment to [ligase ligand moiety].

In some embodiments, R¹⁰ is —C₂₋₅alkyl-O—R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the naphthyl is optionally substituted with —O— or —S—.

In some embodiments, R¹² is H,

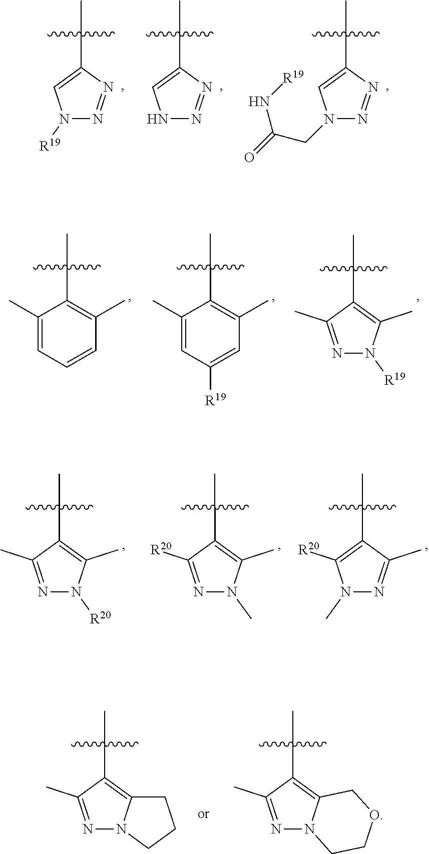

In some embodiments, R²⁰ is Me, —CH₂—O-bromobenzaldehyde, or

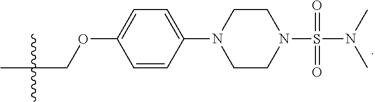 [SMILES: *COc1ccc(N2CCN(S(=O)(=O)N(C)C)CC2)cc1]

In some embodiments, when R⁸ is H, R¹³ is

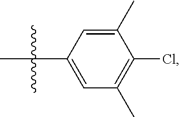 [SMILES: *c1cc(C)c(Cl)c(C)c1]

In some embodiments, R⁸ is H, R¹⁹, methyl, or —CH₂CH₂-morpholine; R⁹ is —C(O)OH or —C(O)NHR¹⁹; R¹⁰ is —C₃H₆O—R¹³ wherein R¹³ is

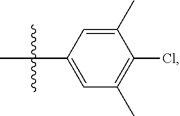 [SMILES: *c1cc(C)c(Cl)c(C)c1]

tetraline, or naphthyl optionally substituted with fluorine;
R¹¹ is H, Cl, F or methyl, and
R¹² is

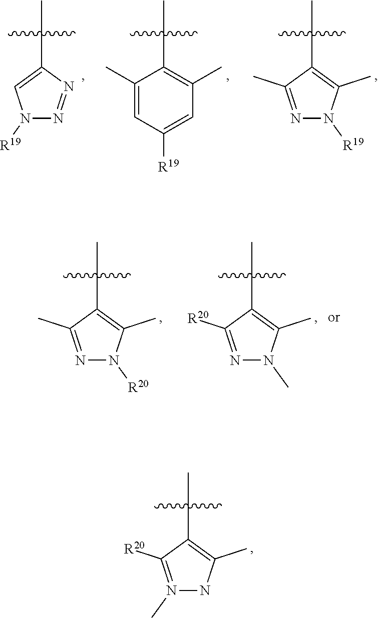

wherein R²⁰ is Me, —CH₂—O-bromobenzaldehyde, or

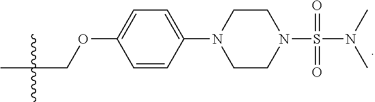 [SMILES: *COc1ccc(N2CCN(S(=O)(=O)N(C)C)CC2)cc1]

In some embodiments, Z₂ is N and [symbol] is a single bond. In other embodiments, Z₂ is C and [symbol] is a double bond.

In some embodiments, R¹¹ is hydrogen. In other embodiments, R¹¹ is halogen or C₁-C₆ alkyl. In some embodiments, R¹¹ is halogen.

In some embodiments, [MCL-1 ligand moiety] is selected from:

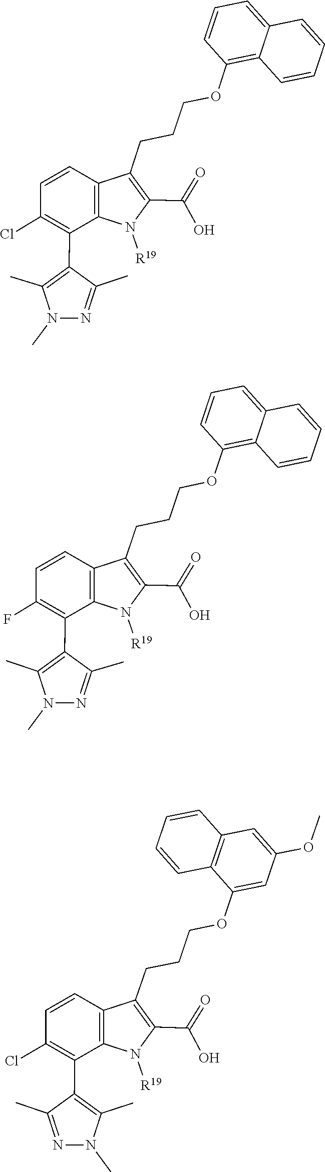

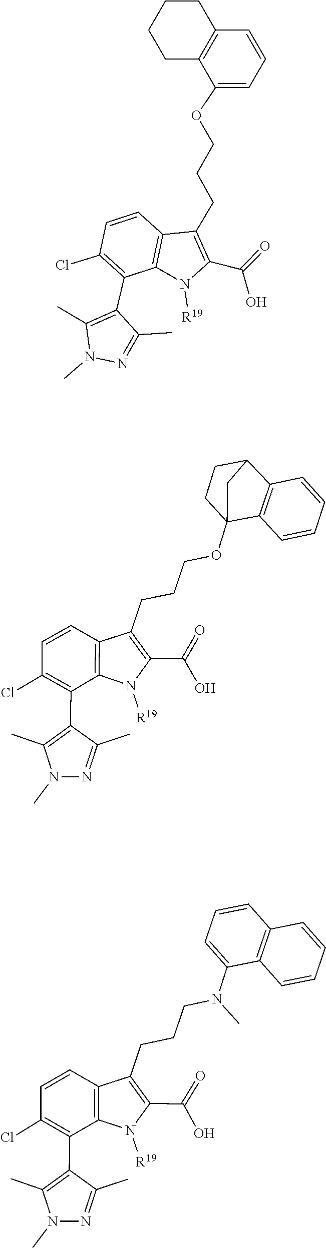

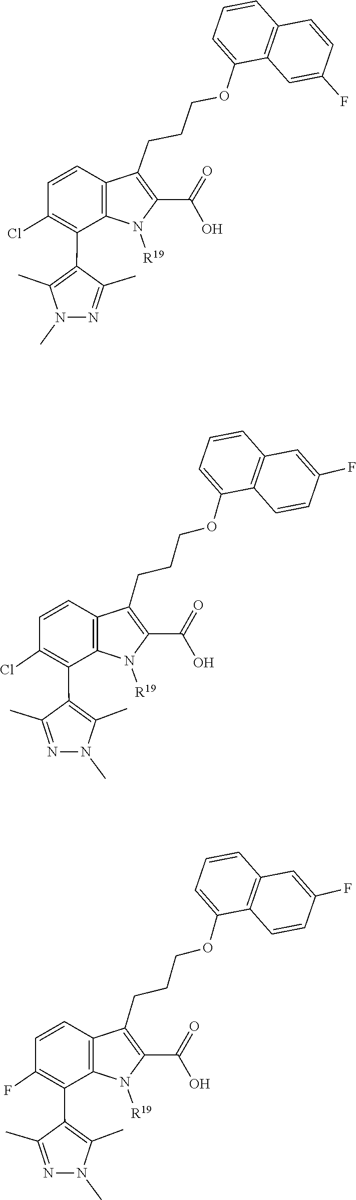

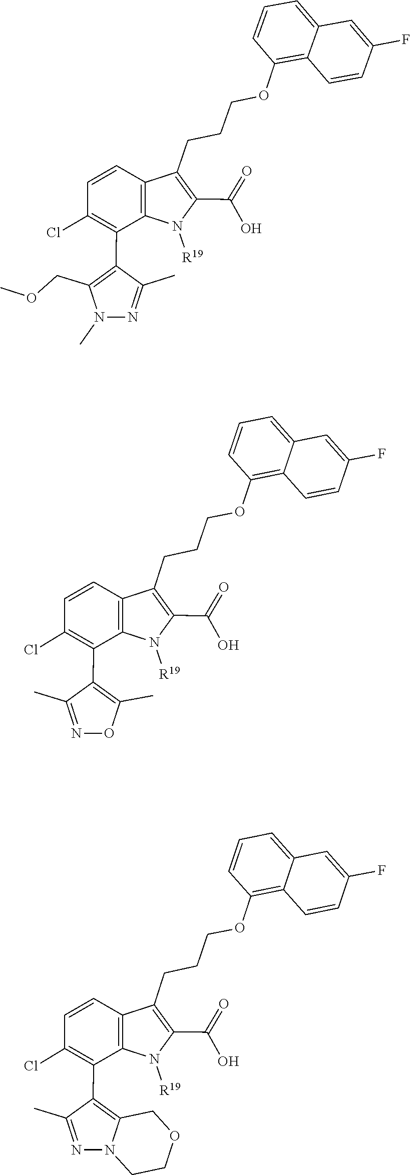

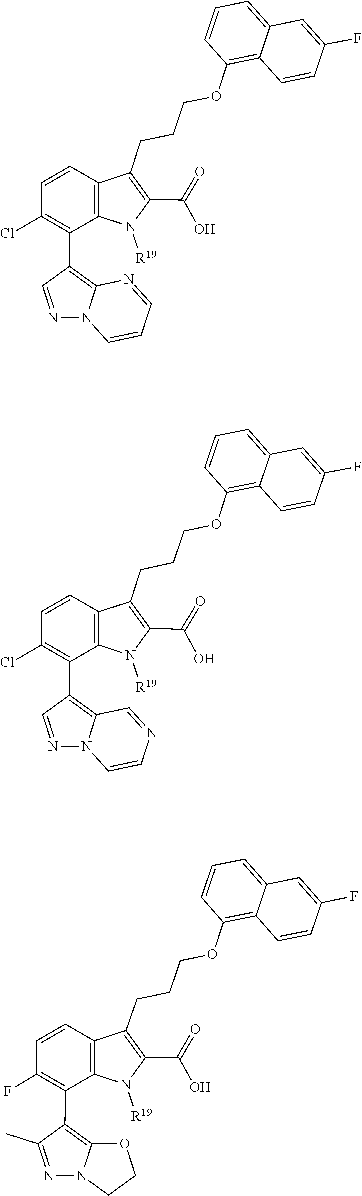

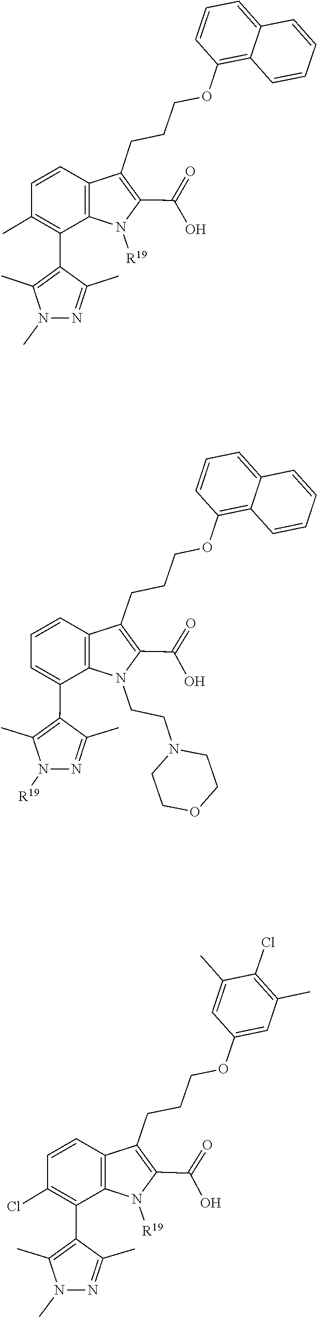

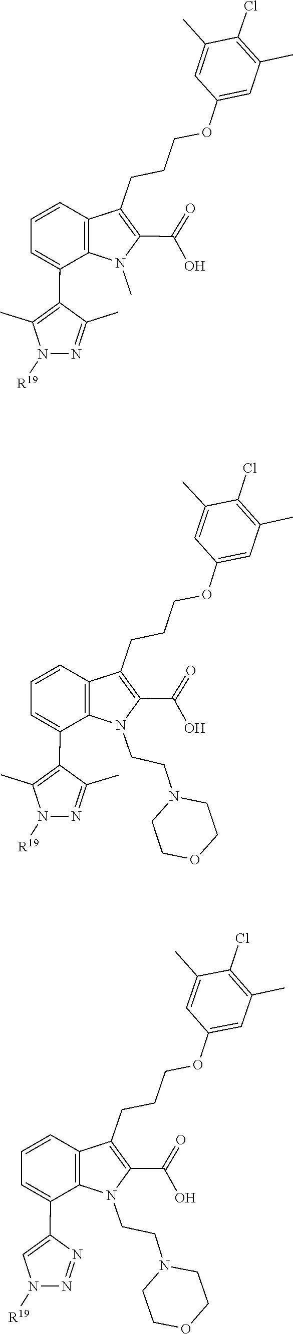

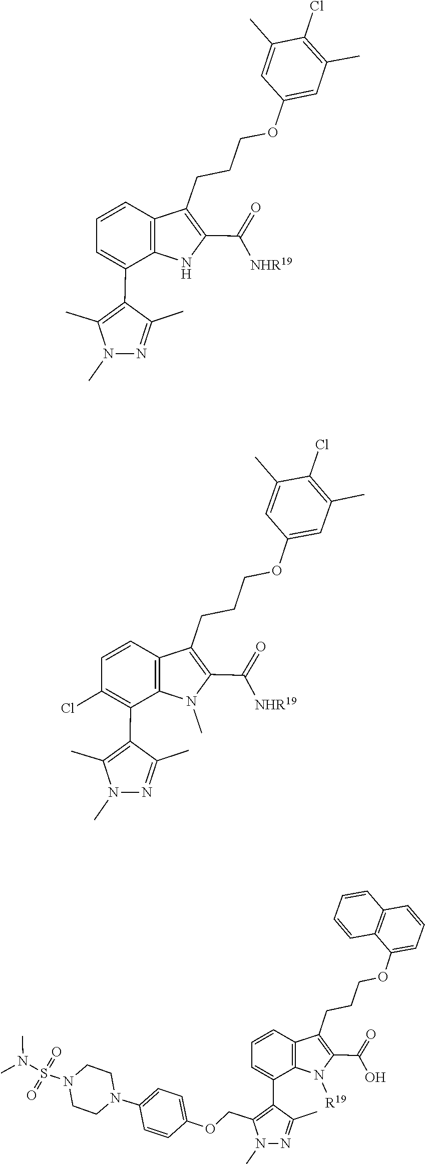

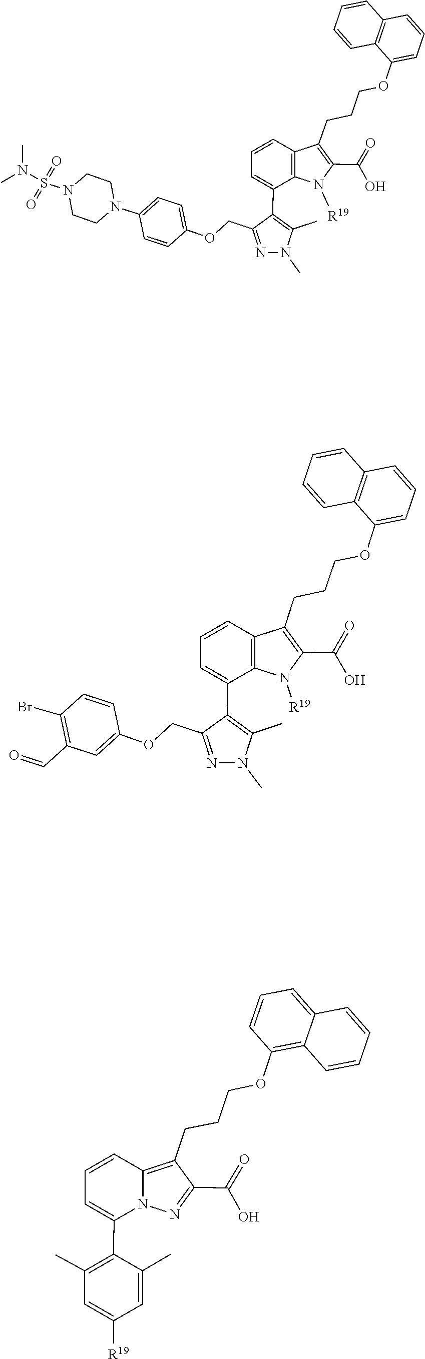

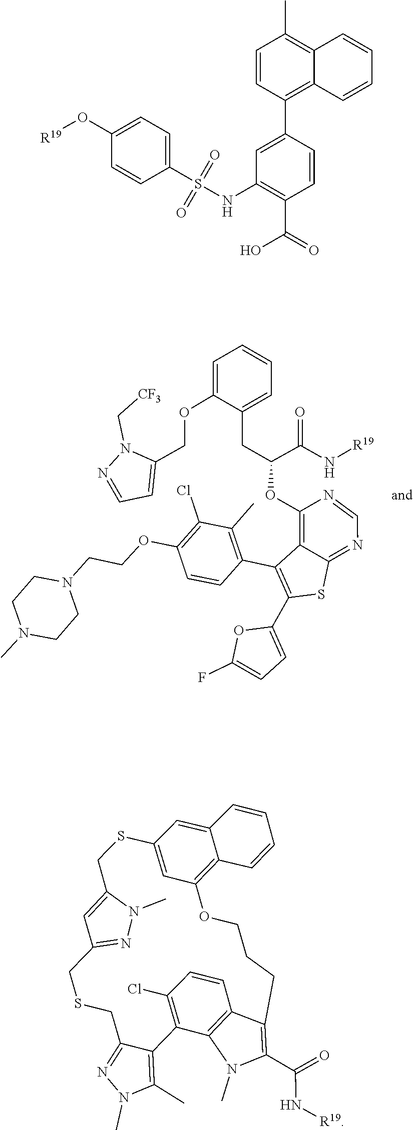

In some embodiments, [MCL-1 ligand moiety] is selected from:

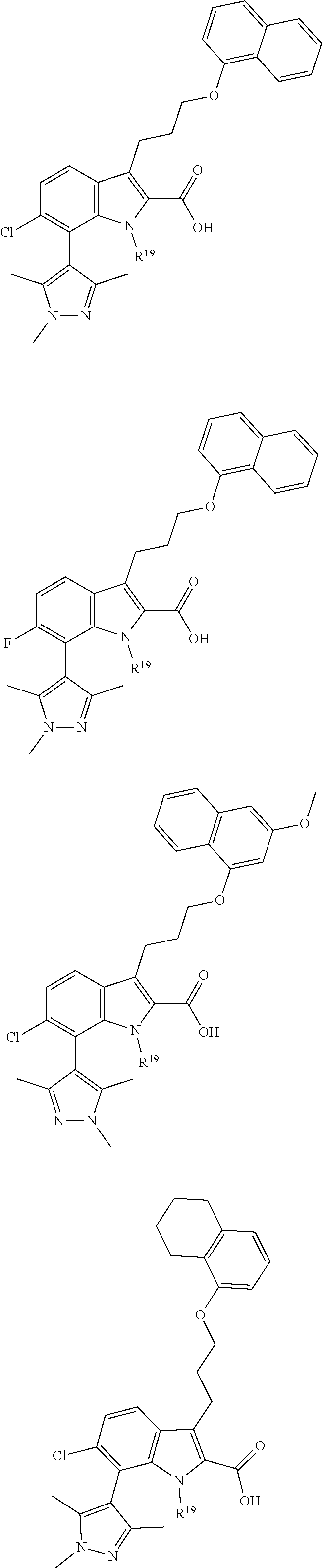

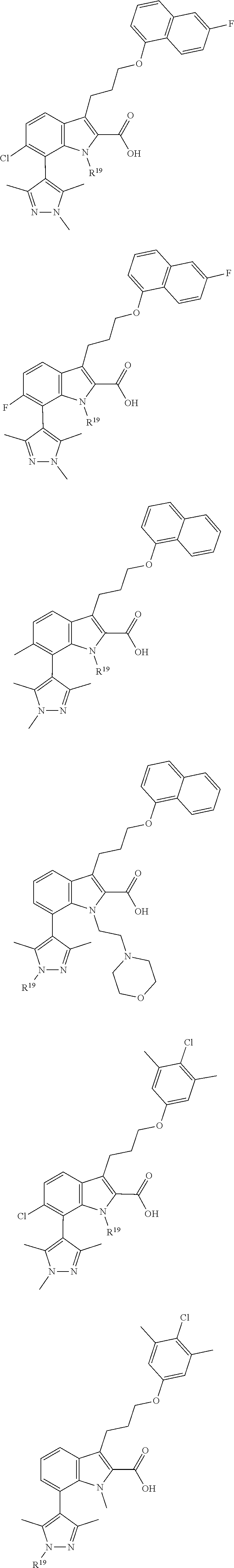

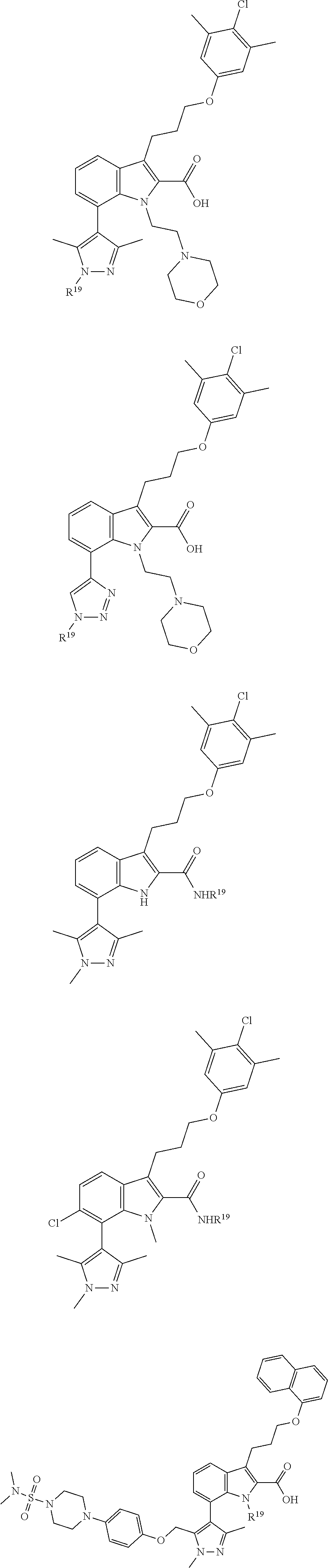

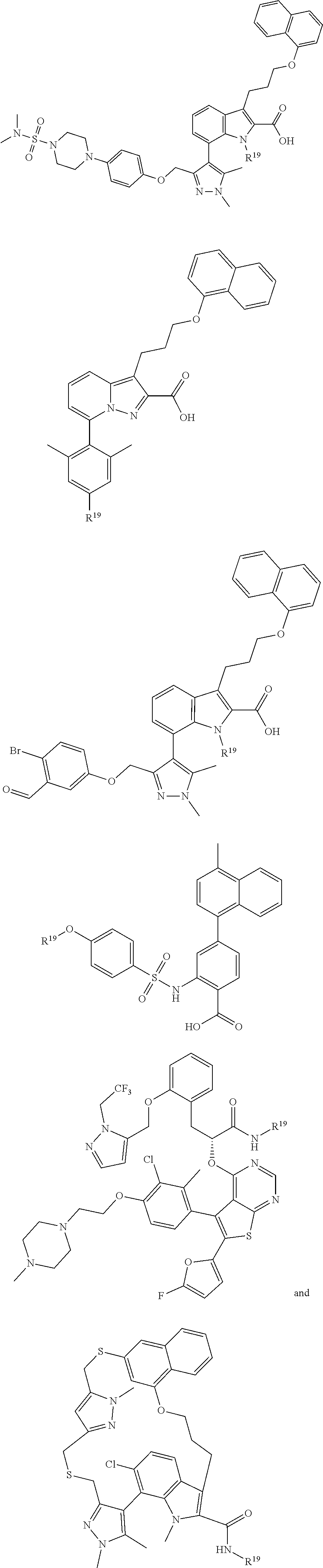

In some embodiments, the compound is selected from:

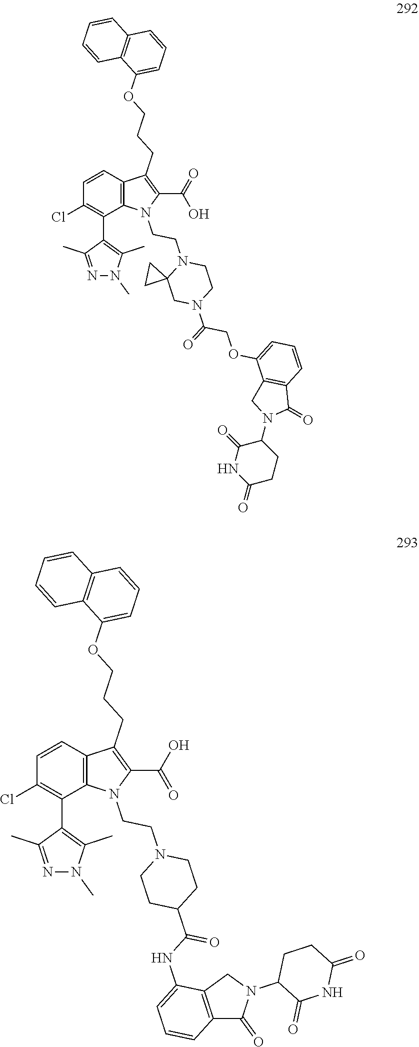 [SMILES: Cc1nn(C)c(C)c1-c1c(Cl)ccc2c(CCCOc3cccc4ccccc34)c(C(=O)O)n(CCN3CCN(C(=O)COc4cccc5c4CN(C4CCC(=O)NC4=O)C5=O)CC34CC4)c12 ; Cc1nn(C)c(C)c1-c1c(Cl)ccc2c(CCCOc3cccc4ccccc34)c(C(=O)O)n(CCN3CCC(C(=O)Nc4cccc5c4CN(C4CCC(=O)NC4=O)C5=O)CC3)c12]

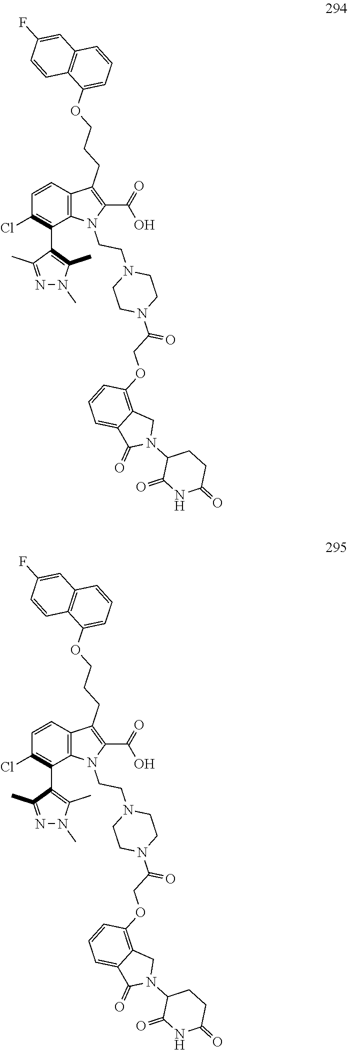

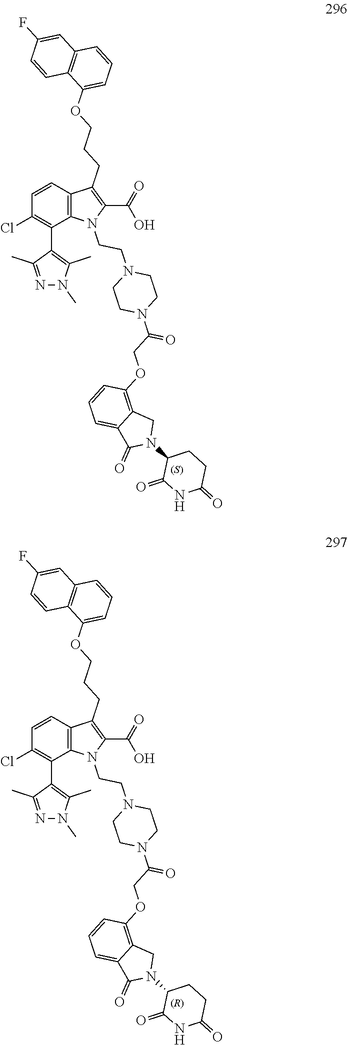

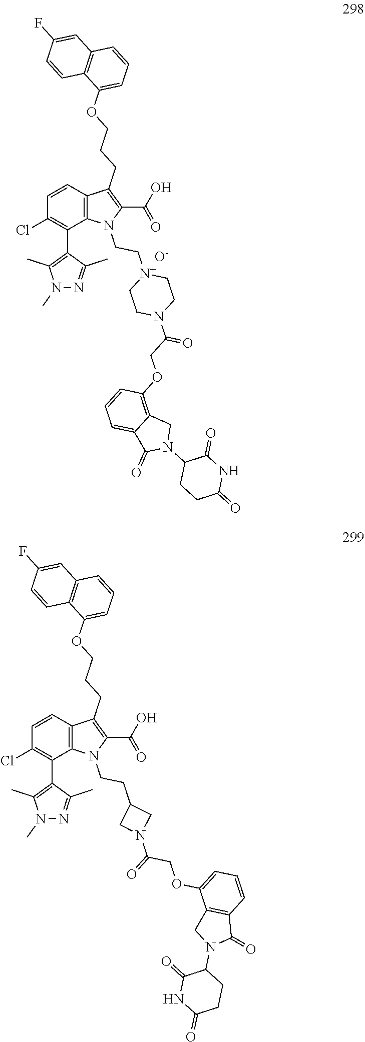

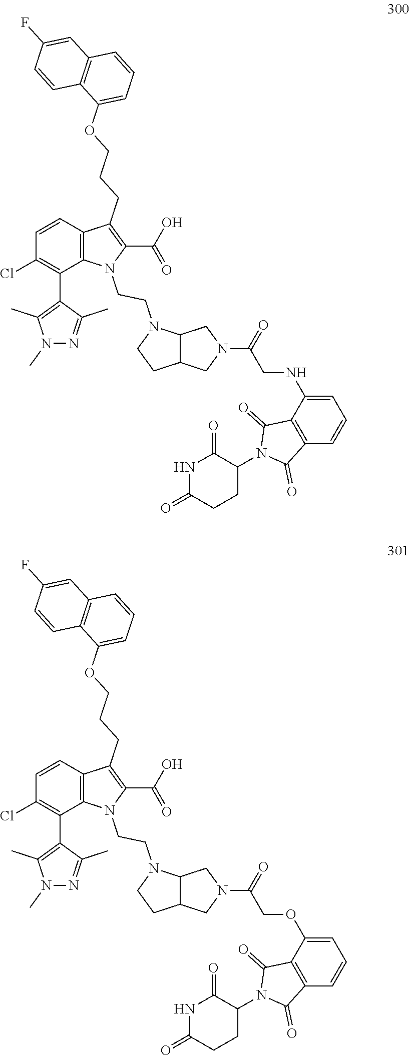 [SMILES: Cc1nn(C)c(C)c1-c1c(Cl)ccc2c(CCCOc3cccc4cc(F)ccc34)c(C(=O)O)n(CCN3CCC4CN(C(=O)CNc5cccc6c5C(=O)N(C5CCC(=O)NC5=O)C6=O)CC43)c12 ; Cc1nn(C)c(C)c1-c1c(Cl)ccc2c(CCCOc3cccc4cc(F)ccc34)c(C(=O)O)n(CCN3CCC4CN(C(=O)COc5cccc6c5C(=O)N(C5CCC(=O)NC5=O)C6=O)CC43)c12]

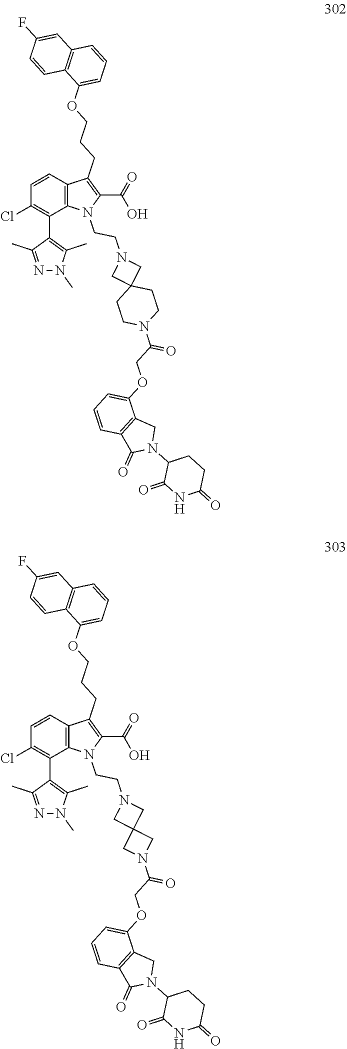 [SMILES: Cc1nn(C)c(C)c1-c1c(Cl)ccc2c(CCCOc3cccc4cc(F)ccc34)c(C(=O)O)n(CCN3CC4(CCN(C(=O)COc5cccc6c5CN(C5CCC(=O)NC5=O)C6=O)CC4)C3)c12 ; Cc1nn(C)c(C)c1-c1c(Cl)ccc2c(CCCOc3cccc4cc(F)ccc34)c(C(=O)O)n(CCN3CC4(C3)CN(C(=O)COc3cccc5c3CN(C3CCC(=O)NC3=O)C5=O)C4)c12]

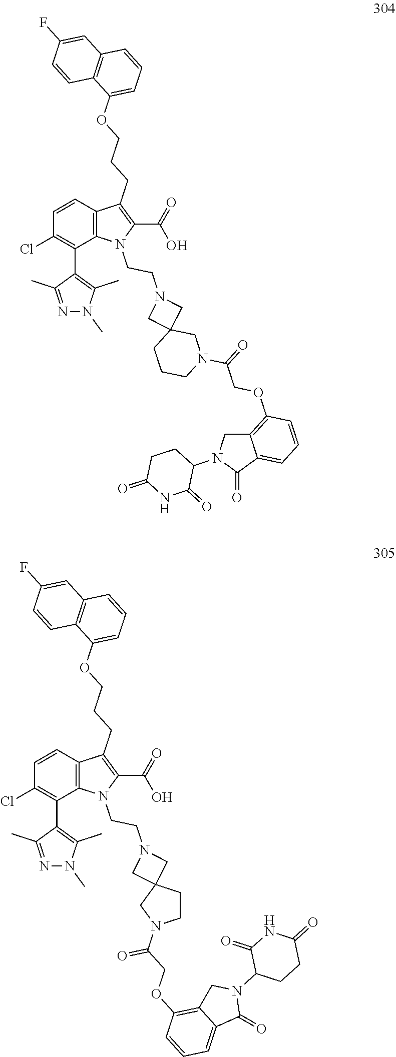 [SMILES: Cc1nn(C)c(C)c1-c1c(Cl)ccc2c(CCCOc3cccc4cc(F)ccc34)c(C(=O)O)n(CCN3CC4(CCCN(C(=O)COc5cccc6c5CN(C5CCC(=O)NC5=O)C6=O)C4)C3)c12 ; Cc1nn(C)c(C)c1-c1c(Cl)ccc2c(CCCOc3cccc4cc(F)ccc34)c(C(=O)O)n(CCN3CC4(CCN(C(=O)COc5cccc6c5CN(C5CCC(=O)NC5=O)C6=O)C4)C3)c12]

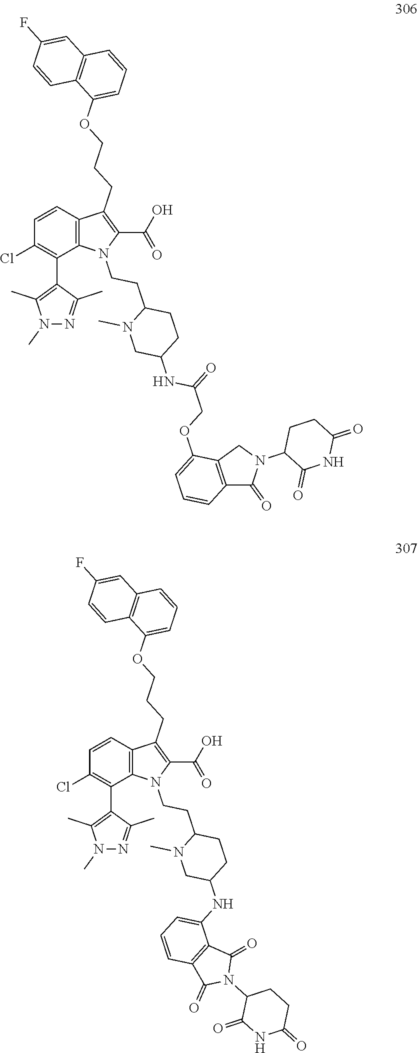 [SMILES: Cc1nn(C)c(C)c1-c1c(Cl)ccc2c(CCCOc3cccc4cc(F)ccc34)c(C(=O)O)n(CCC3CCC(NC(=O)COc4cccc5c4CN(C4CCC(=O)NC4=O)C5=O)CN3C)c12 ; Cc1nn(C)c(C)c1-c1c(Cl)ccc2c(CCCOc3cccc4cc(F)ccc34)c(C(=O)O)n(CCC3CCC(Nc4cccc5c4C(=O)N(C4CCC(=O)NC4=O)C5=O)CN3C)c12]

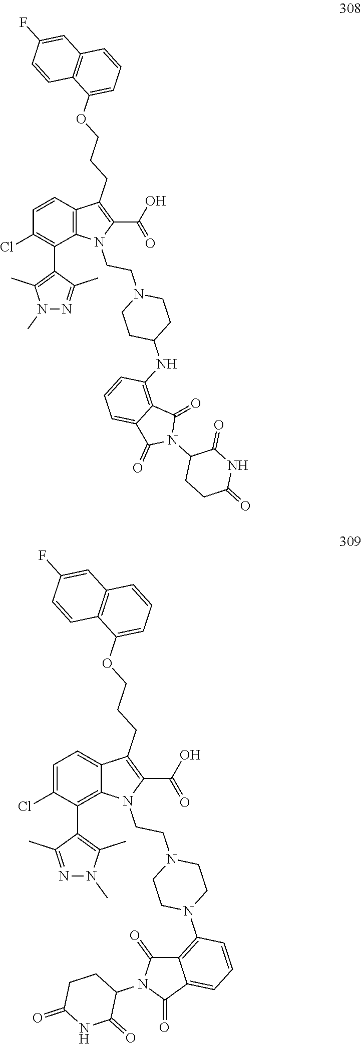 [SMILES: Cc1nn(C)c(C)c1-c1c(Cl)ccc2c(CCCOc3cccc4cc(F)ccc34)c(C(=O)O)n(CCN3CCC(Nc4cccc5c4C(=O)N(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(c4cccc5c4C(=O)N(C4CCC(=O)NC4=O)C5=O)CC3)c12]

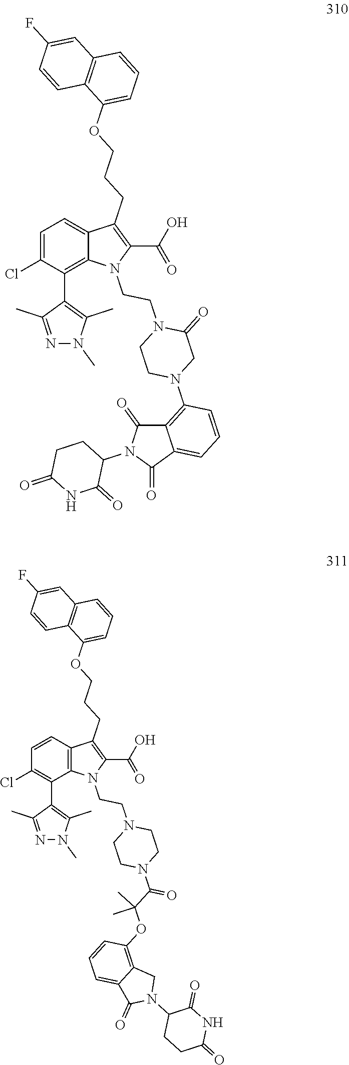 [SMILES: Cc1nn(C)c(C)c1-c1c(Cl)ccc2c(CCCOc3cccc4cc(F)ccc34)c(C(=O)O)n(CCN3CCN(c4cccc5c4C(=O)N(C4CCC(=O)NC4=O)C5=O)CC3=O)c12 ; Cc1nn(C)c(C)c1-c1c(Cl)ccc2c(CCCOc3cccc4cc(F)ccc34)c(C(=O)O)n(CCN3CCN(C(=O)C(C)(C)Oc4cccc5c4CN(C4CCC(=O)NC4=O)C5=O)CC3)c12]

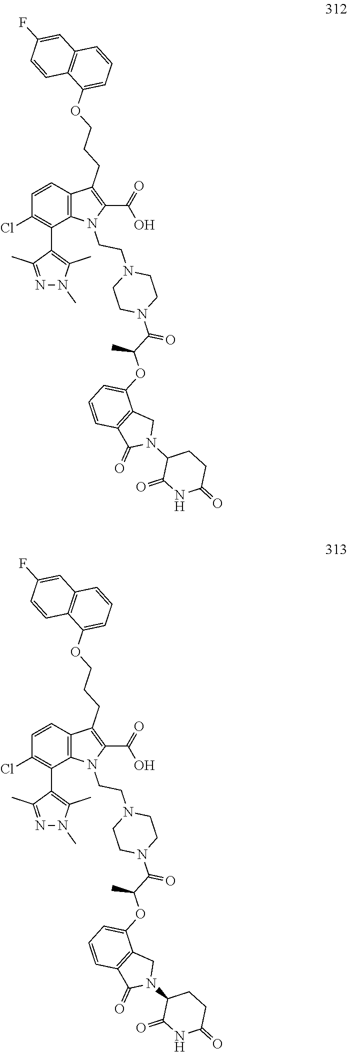 [SMILES: Cc1nn(C)c(C)c1-c1c(Cl)ccc2c(CCCOc3cccc4cc(F)ccc34)c(C(=O)O)n(CCN3CCN(C(=O)[C@H](C)Oc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)[C@H](C)Oc4cccc5c4CN([C@H]4CCC(=O)NC4=O)C5=O)CC3)c12]

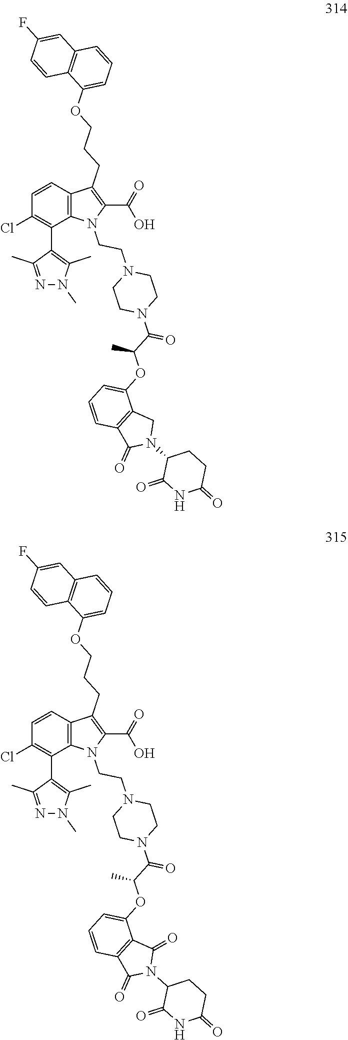 [SMILES: Cc1nn(C)c(C)c1-c1c(Cl)ccc2c(CCCOc3cccc4cc(F)ccc34)c(C(=O)O)n(CCN3CCN(C(=O)[C@H](C)Oc4cccc5c4CN([C@@H]4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)[C@@H](C)Oc4cccc5c4C(=O)N(C4CCC(=O)NC4=O)C5=O)CC3)c12]

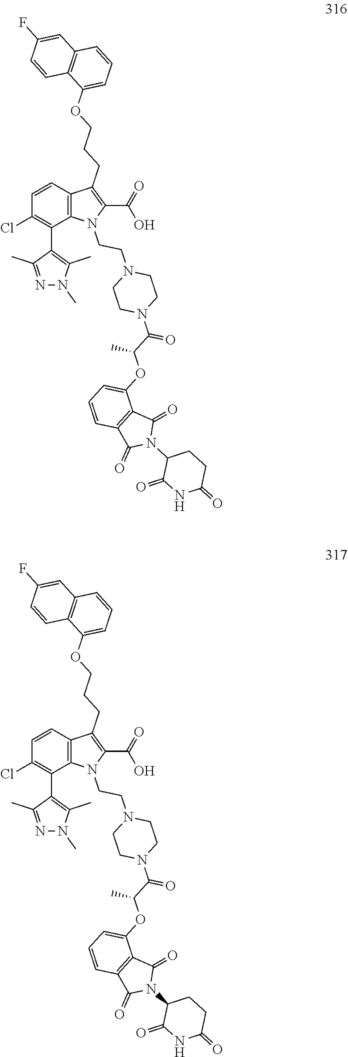 [SMILES: Cc1nn(C)c(C)c1-c1c(Cl)ccc2c(CCCOc3cccc4cc(F)ccc34)c(C(=O)O)n(CCN3CCN(C(=O)[C@@H](C)Oc4cccc5c4C(=O)N(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)[C@@H](C)Oc4cccc5c4C(=O)N([C@H]4CCC(=O)NC4=O)C5=O)CC3)c12]

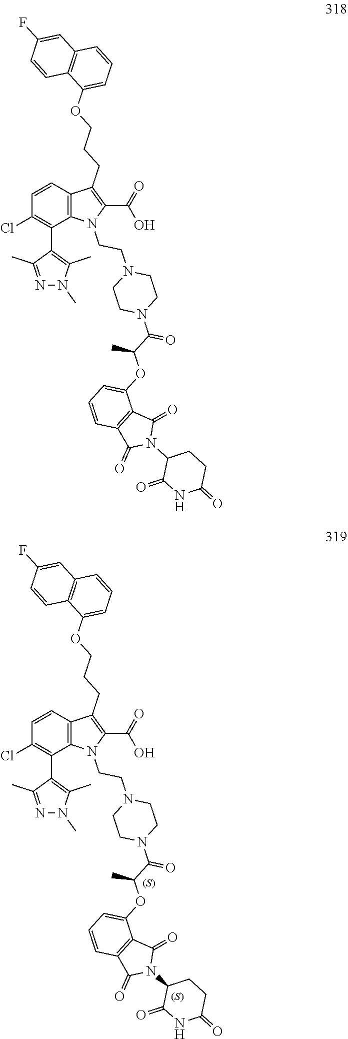

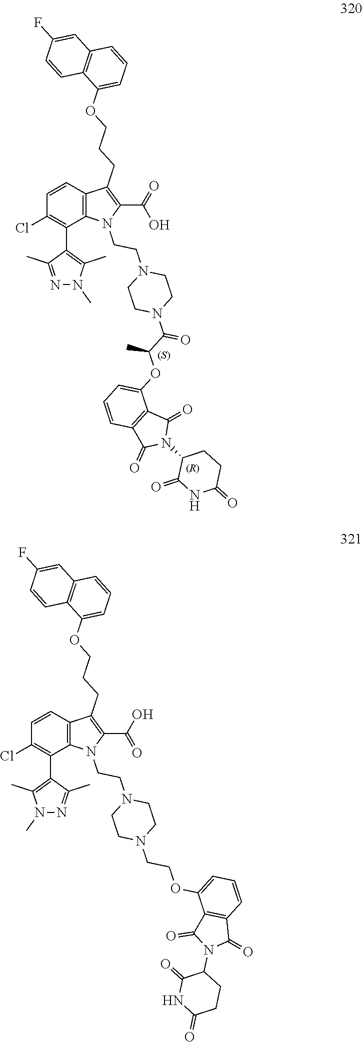

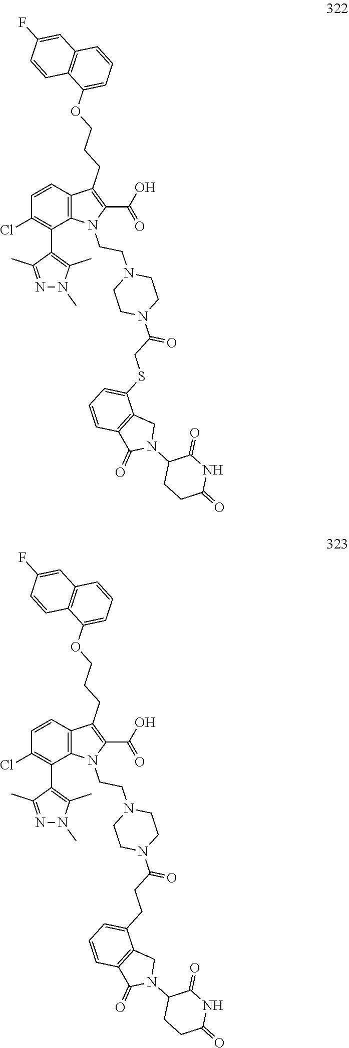 [SMILES: Cc1nn(C)c(C)c1-c1c(Cl)ccc2c(CCCOc3cccc4cc(F)ccc34)c(C(=O)O)n(CCN3CCN(C(=O)CSc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)CCc4cccc5c4CN(C4CCC(=O)NC4=O)C5=O)CC3)c12]

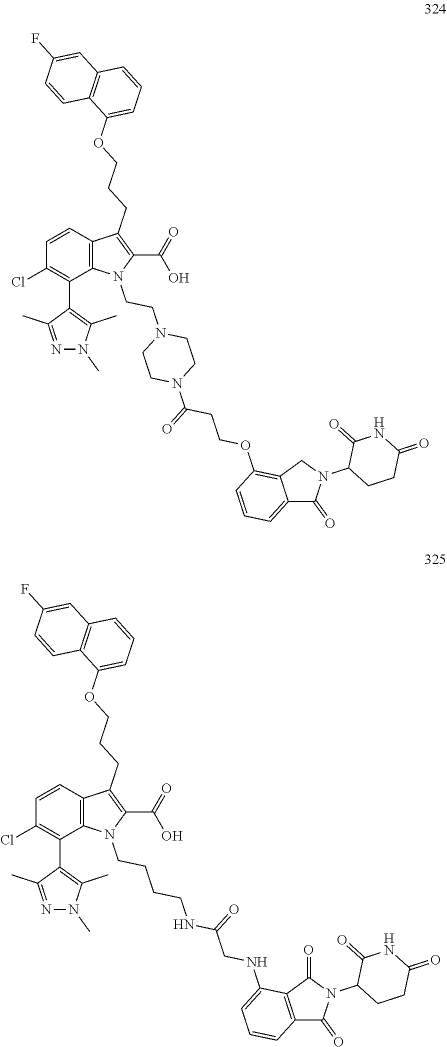 [SMILES: Cc1nn(C)c(C)c1-c1c(Cl)ccc2c(CCCOc3cccc4cc(F)ccc34)c(C(=O)O)n(CCN3CCN(C(=O)CCOc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCCCNC(=O)CNc3cccc4c3C(=O)N(C3CCC(=O)NC3=O)C4=O)c12]

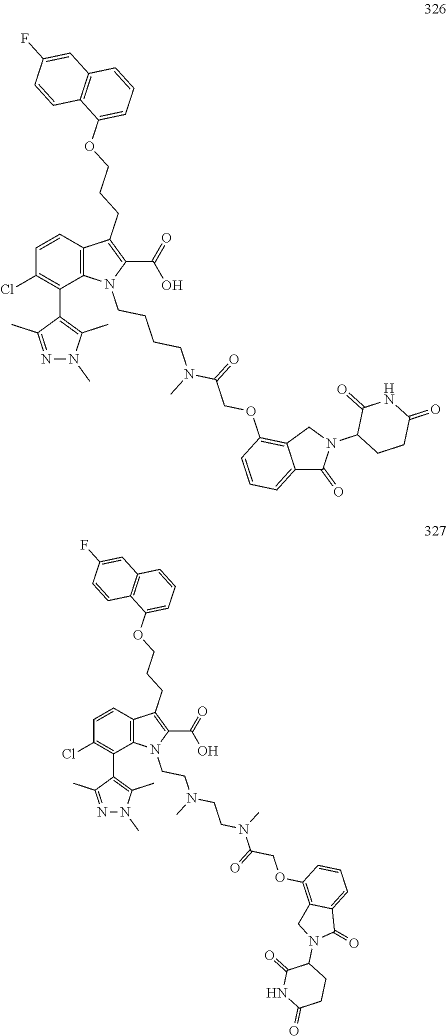 [SMILES: Cc1nn(C)c(C)c1-c1c(Cl)ccc2c(CCCOc3cccc4cc(F)ccc34)c(C(=O)O)n(CCCCN(C)C(=O)COc3cccc4c3CN(C3CCC(=O)NC3=O)C4=O)c12 ; Cc1nn(C)c(C)c1-c1c(Cl)ccc2c(CCCOc3cccc4cc(F)ccc34)c(C(=O)O)n(CCN(C)CCN(C)C(=O)COc3cccc4c3CN(C3CCC(=O)NC3=O)C4=O)c12]

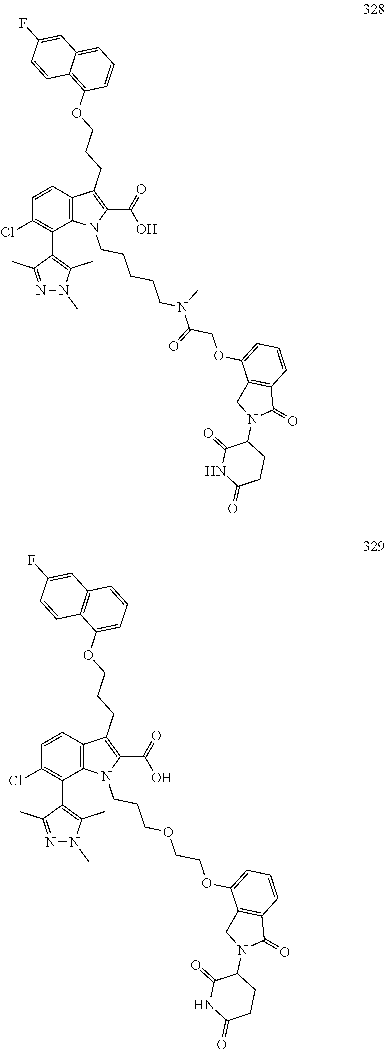 [SMILES: Cc1nn(C)c(C)c1-c1c(Cl)ccc2c(CCCOc3cccc4cc(F)ccc34)c(C(=O)O)n(CCCCCN(C)C(=O)COc3cccc4c3CN(C3CCC(=O)NC3=O)C4=O)c12 ; Cc1nn(C)c(C)c1-c1c(Cl)ccc2c(CCCOc3cccc4cc(F)ccc34)c(C(=O)O)n(CCCOCCOc3cccc4c3CN(C3CCC(=O)NC3=O)C4=O)c12]

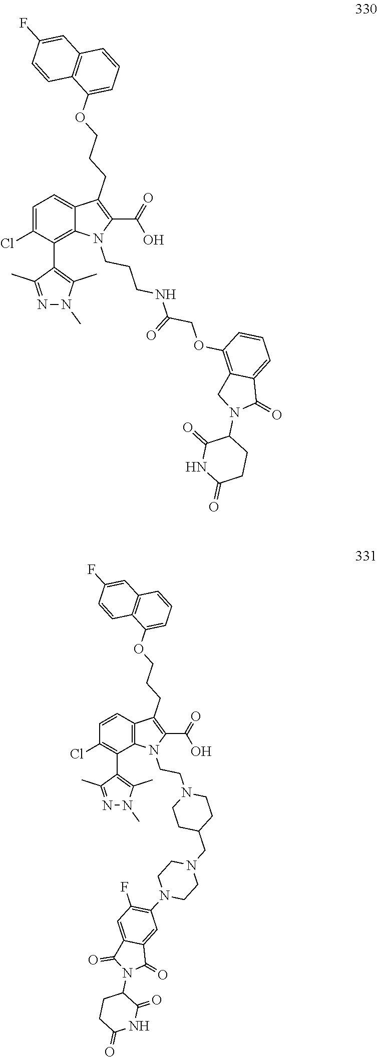 [SMILES: Cc1nn(C)c(C)c1-c1c(Cl)ccc2c(CCCOc3cccc4cc(F)ccc34)c(C(=O)O)n(CCCNC(=O)COc3cccc4c3CN(C3CCC(=O)NC3=O)C4=O)c12 ; Cc1nn(C)c(C)c1-c1c(Cl)ccc2c(CCCOc3cccc4cc(F)ccc34)c(C(=O)O)n(CCN3CCC(CN4CCN(c5cc6c(cc5F)C(=O)N(C5CCC(=O)NC5=O)C6=O)CC4)CC3)c12]

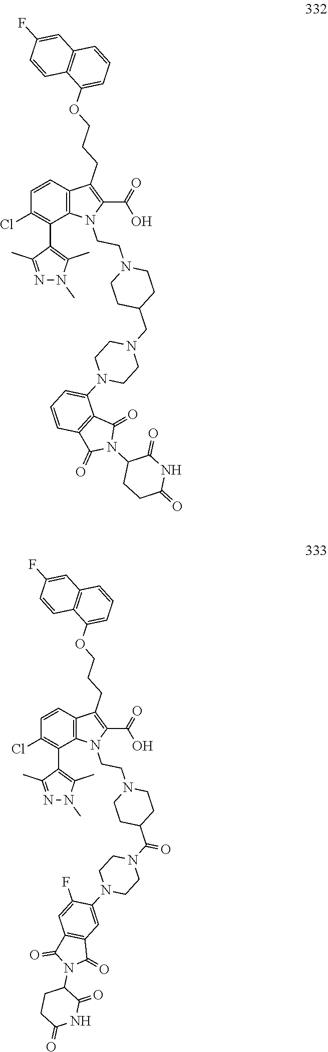 [SMILES: Cc1nn(C)c(C)c1-c1c(Cl)ccc2c(CCCOc3cccc4cc(F)ccc34)c(C(=O)O)n(CCN3CCC(CN4CCN(c5cccc6c5C(=O)N(C5CCC(=O)NC5=O)C6=O)CC4)CC3)c12 ; Cc1nn(C)c(C)c1-c1c(Cl)ccc2c(CCCOc3cccc4cc(F)ccc34)c(C(=O)O)n(CCN3CCC(C(=O)N4CCN(c5cc6c(cc5F)C(=O)N(C5CCC(=O)NC5=O)C6=O)CC4)CC3)c12]

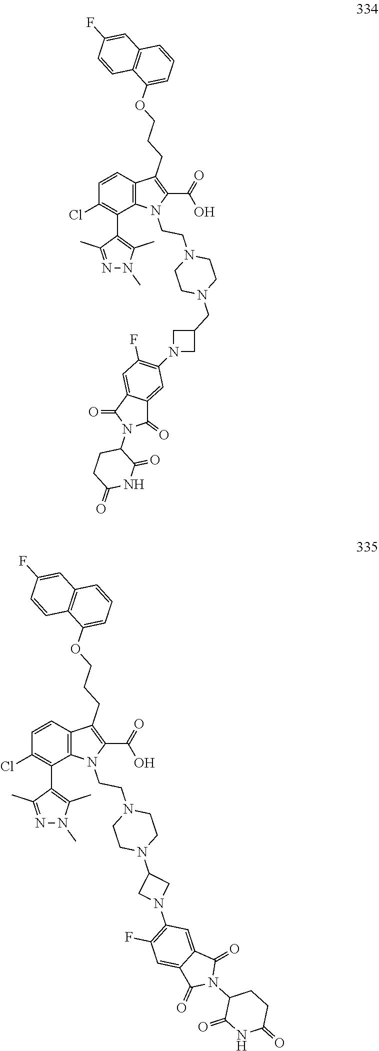 [SMILES: Cc1nn(C)c(C)c1-c1c(Cl)ccc2c(CCCOc3cccc4cc(F)ccc34)c(C(=O)O)n(CCN3CCN(CC4CN(c5cc6c(cc5F)C(=O)N(C5CCC(=O)NC5=O)C6=O)C4)CC3)c12 ; Cc1nn(C)c(C)c1-c1c(Cl)ccc2c(CCCOc3cccc4cc(F)ccc34)c(C(=O)O)n(CCN3CCN(C4CN(c5cc6c(cc5F)C(=O)N(C5CCC(=O)NC5=O)C6=O)C4)CC3)c12]

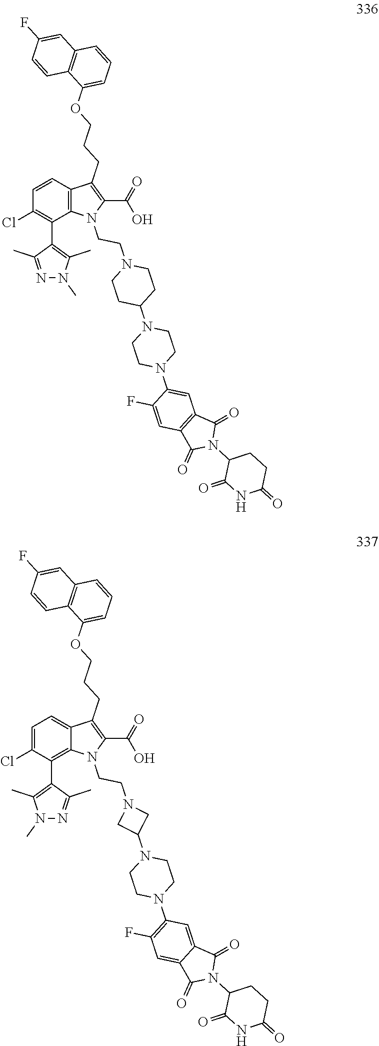 [SMILES: Cc1nn(C)c(C)c1-c1c(Cl)ccc2c(CCCOc3cccc4cc(F)ccc34)c(C(=O)O)n(CCN3CCC(N4CCN(c5cc6c(cc5F)C(=O)N(C5CCC(=O)NC5=O)C6=O)CC4)CC3)c12 ; Cc1nn(C)c(C)c1-c1c(Cl)ccc2c(CCCOc3cccc4cc(F)ccc34)c(C(=O)O)n(CCN3CC(N4CCN(c5cc6c(cc5F)C(=O)N(C5CCC(=O)NC5=O)C6=O)CC4)C3)c12]

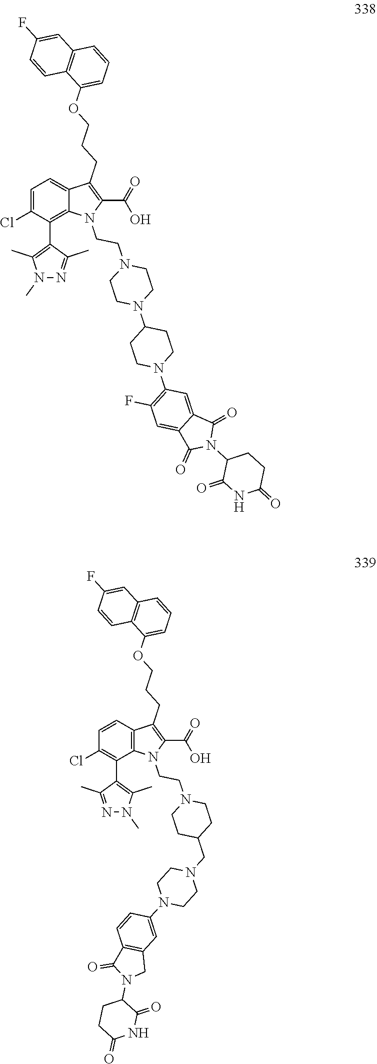 [SMILES: Cc1nn(C)c(C)c1-c1c(Cl)ccc2c(CCCOc3cccc4cc(F)ccc34)c(C(=O)O)n(CCN3CCN(C4CCN(c5cc6c(cc5F)C(=O)N(C5CCC(=O)NC5=O)C6=O)CC4)CC3)c12 ; Cc1nn(C)c(C)c1-c1c(Cl)ccc2c(CCCOc3cccc4cc(F)ccc34)c(C(=O)O)n(CCN3CCC(CN4CCN(c5ccc6c(c5)CN(C5CCC(=O)NC5=O)C6=O)CC4)CC3)c12]

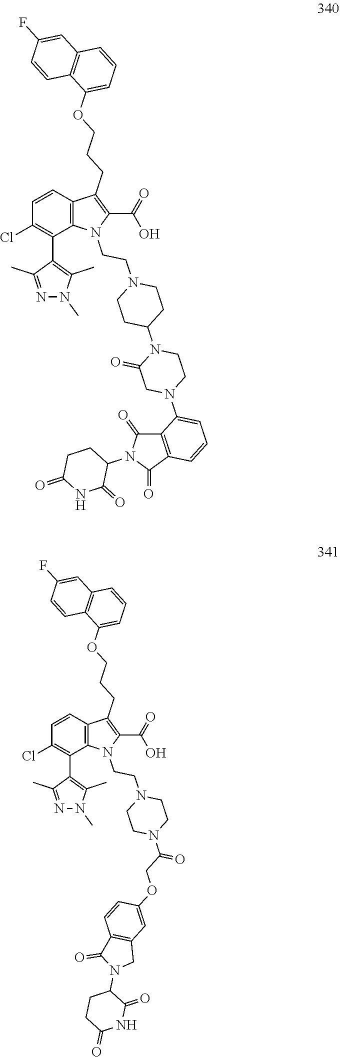 [SMILES: Cc1nn(C)c(C)c1-c1c(Cl)ccc2c(CCCOc3cccc4cc(F)ccc34)c(C(=O)O)n(CCN3CCC(N4CCN(c5cccc6c5C(=O)N(C5CCC(=O)NC5=O)C6=O)CC4=O)CC3)c12 ; Cc1nn(C)c(C)c1-c1c(Cl)ccc2c(CCCOc3cccc4cc(F)ccc34)c(C(=O)O)n(CCN3CCN(C(=O)COc4ccc5c(c4)CN(C4CCC(=O)NC4=O)C5=O)CC3)c12]

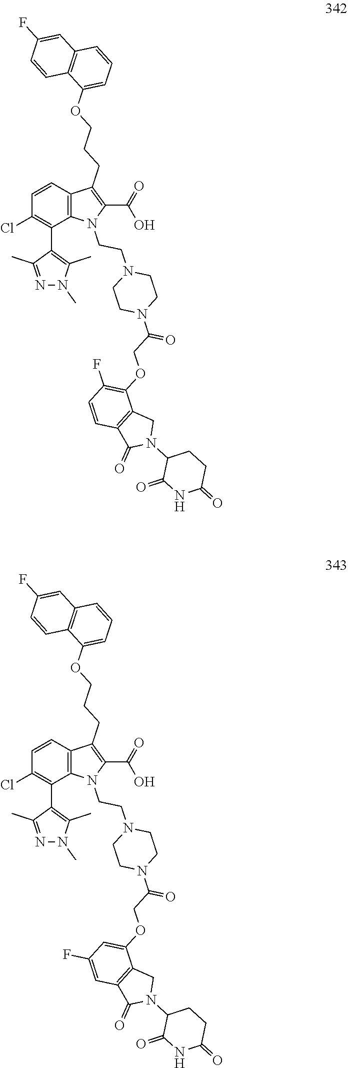 [SMILES: Cc1nn(C)c(C)c1-c1c(Cl)ccc2c(CCCOc3cccc4cc(F)ccc34)c(C(=O)O)n(CCN3CCN(C(=O)COc4c(F)ccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4cc(F)cc5c4CN(C4CCC(=O)NC4=O)C5=O)CC3)c12]

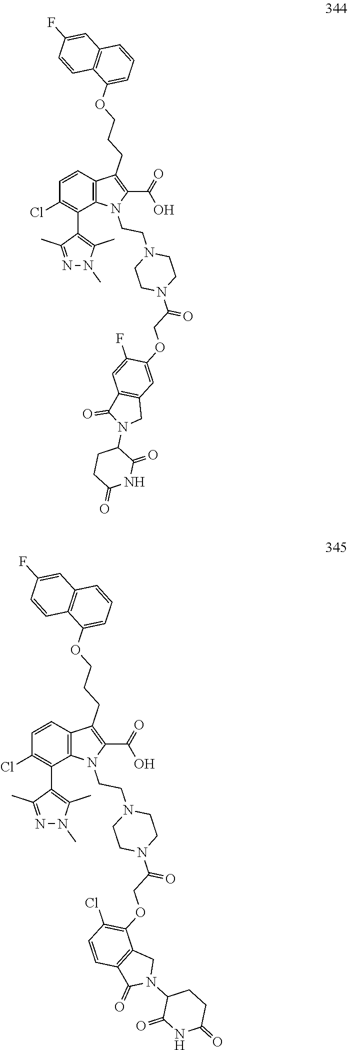 [SMILES: Cc1nn(C)c(C)c1-c1c(Cl)ccc2c(CCCOc3cccc4cc(F)ccc34)c(C(=O)O)n(CCN3CCN(C(=O)COc4cc5c(cc4F)C(=O)N(C4CCC(=O)NC4=O)C5)CC3)c12 ; Cc1nn(C)c(C)c1-c1c(Cl)ccc2c(CCCOc3cccc4cc(F)ccc34)c(C(=O)O)n(CCN3CCN(C(=O)COc4c(Cl)ccc5c4CN(C4CCC(=O)NC4=O)C5=O)CC3)c12]

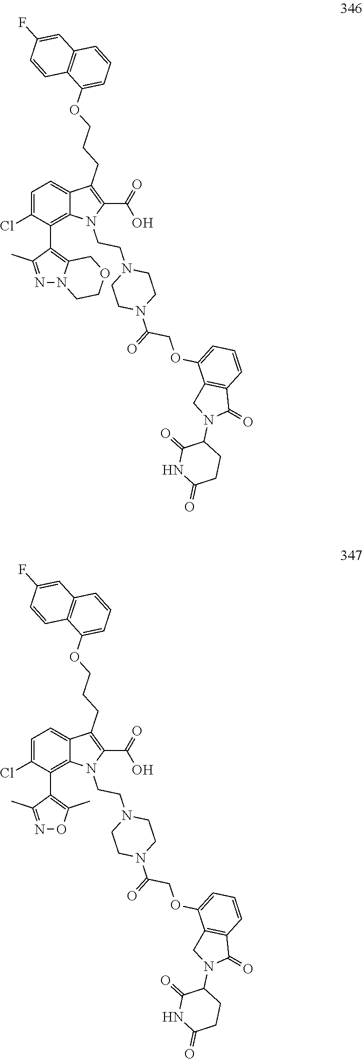 [SMILES: Cc1nn2c(c1-c1c(Cl)ccc3c(CCCOc4cccc5cc(F)ccc45)c(C(=O)O)n(CCN4CCN(C(=O)COc5cccc6c5CN(C5CCC(=O)NC5=O)C6=O)CC4)c13)COCC2 ; Cc1noc(C)c1-c1c(Cl)ccc2c(CCCOc3cccc4cc(F)ccc34)c(C(=O)O)n(CCN3CCN(C(=O)COc4cccc5c4CN(C4CCC(=O)NC4=O)C5=O)CC3)c12]

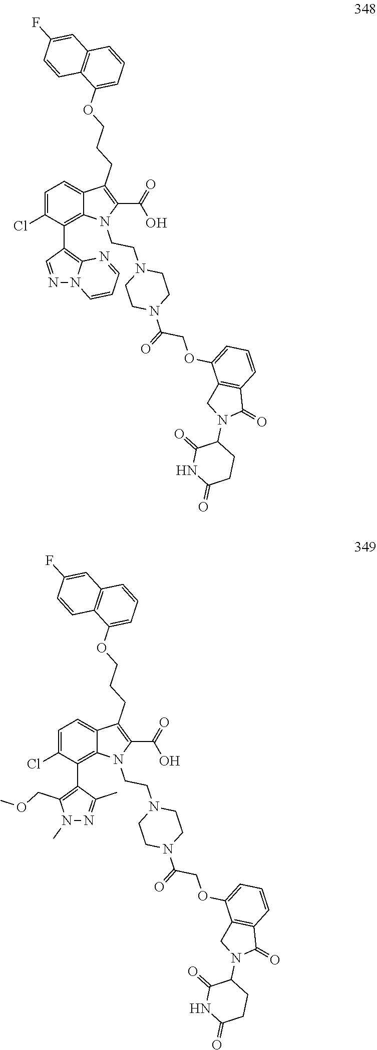 [SMILES: O=C1CCC(N2Cc3c(OCC(=O)N4CCN(CCn5c(C(=O)O)c(CCCOc6cccc7cc(F)ccc67)c6ccc(Cl)c(-c7cnn8cccnc78)c65)CC4)cccc3C2=O)C(=O)N1 ; COCc1c(-c2c(Cl)ccc3c(CCCOc4cccc5cc(F)ccc45)c(C(=O)O)n(CCN4CCN(C(=O)COc5cccc6c5CN(C5CCC(=O)NC5=O)C6=O)CC4)c23)c(C)nn1C]

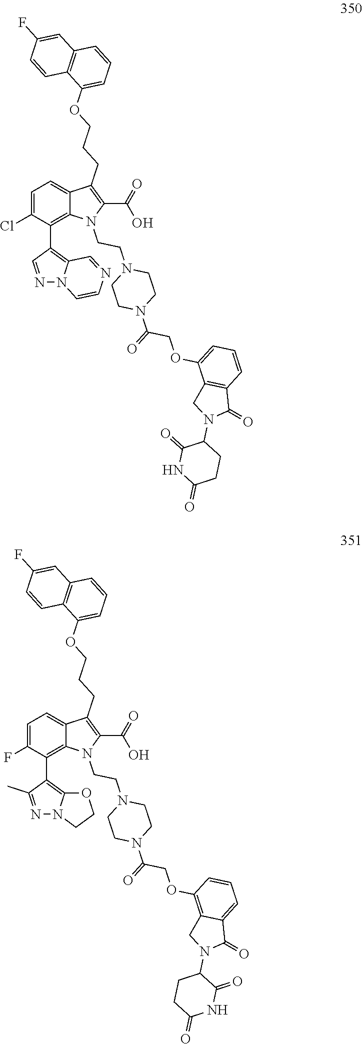 [SMILES: O=C1CCC(N2Cc3c(OCC(=O)N4CCN(CCn5c(C(=O)O)c(CCCOc6cccc7cc(F)ccc67)c6ccc(Cl)c(-c7cnn8ccncc78)c65)CC4)cccc3C2=O)C(=O)N1 ; Cc1nn2c(c1-c1c(F)ccc3c(CCCOc4cccc5cc(F)ccc45)c(C(=O)O)n(CCN4CCN(C(=O)COc5cccc6c5CN(C5CCC(=O)NC5=O)C6=O)CC4)c13)OCC2]

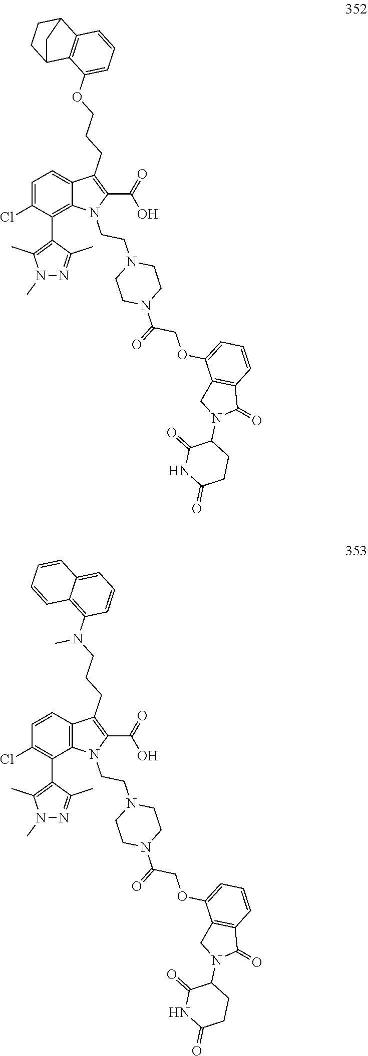 [SMILES: Cc1nn(C)c(C)c1-c1c(Cl)ccc2c(CCCOc3cccc4c3C3CCC4C3)c(C(=O)O)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCN(C)c3cccc4ccccc34)c(C(=O)O)n(CCN3CCN(C(=O)COc4cccc5c4CN(C4CCC(=O)NC4=O)C5=O)CC3)c12]

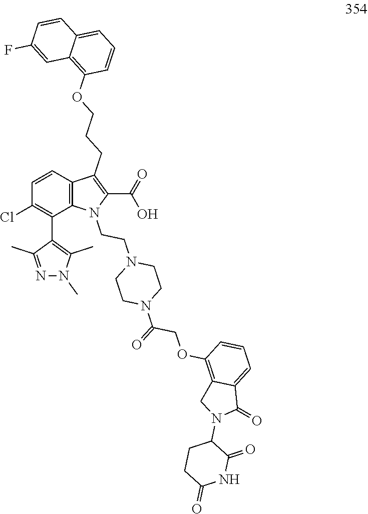 [SMILES: Cc1nn(C)c(C)c1-c1c(Cl)ccc2c(CCCOc3cccc4ccc(F)cc34)c(C(=O)O)n(CCN3CCN(C(=O)COc4cccc5c4CN(C4CCC(=O)NC4=O)C5=O)CC3)c12]

In some embodiments, the compound is selected from:

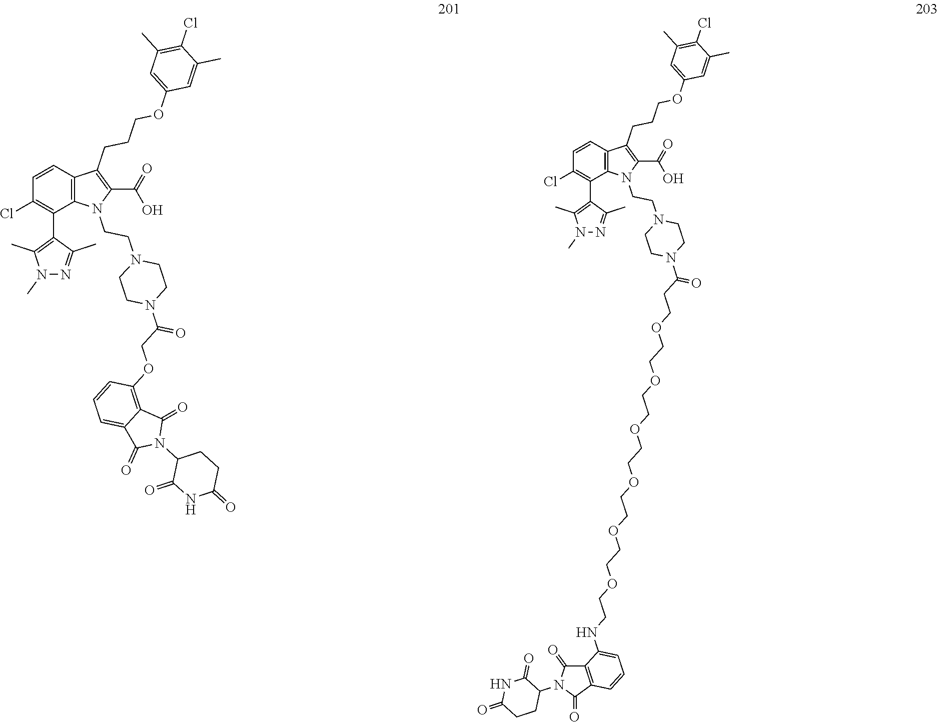 [SMILES: Cc1cc(OCCCc2c(C(=O)O)n(CCN3CCN(C(=O)COc4cccc5c4C(=O)N(C4CCC(=O)NC4=O)C5=O)CC3)c3c(-c4c(C)nn(C)c4C)c(Cl)ccc23)cc(C)c1Cl ; Cc1cc(OCCCc2c(C(=O)O)n(CCN3CCN(C(=O)CCOCCOCCOCCOCCOCCOCCNc4cccc5c4C(=O)N(C4CCC(=O)NC4=O)C5=O)CC3)c3c(-c4c(C)nn(C)c4C)c(Cl)ccc23)cc(C)c1Cl]

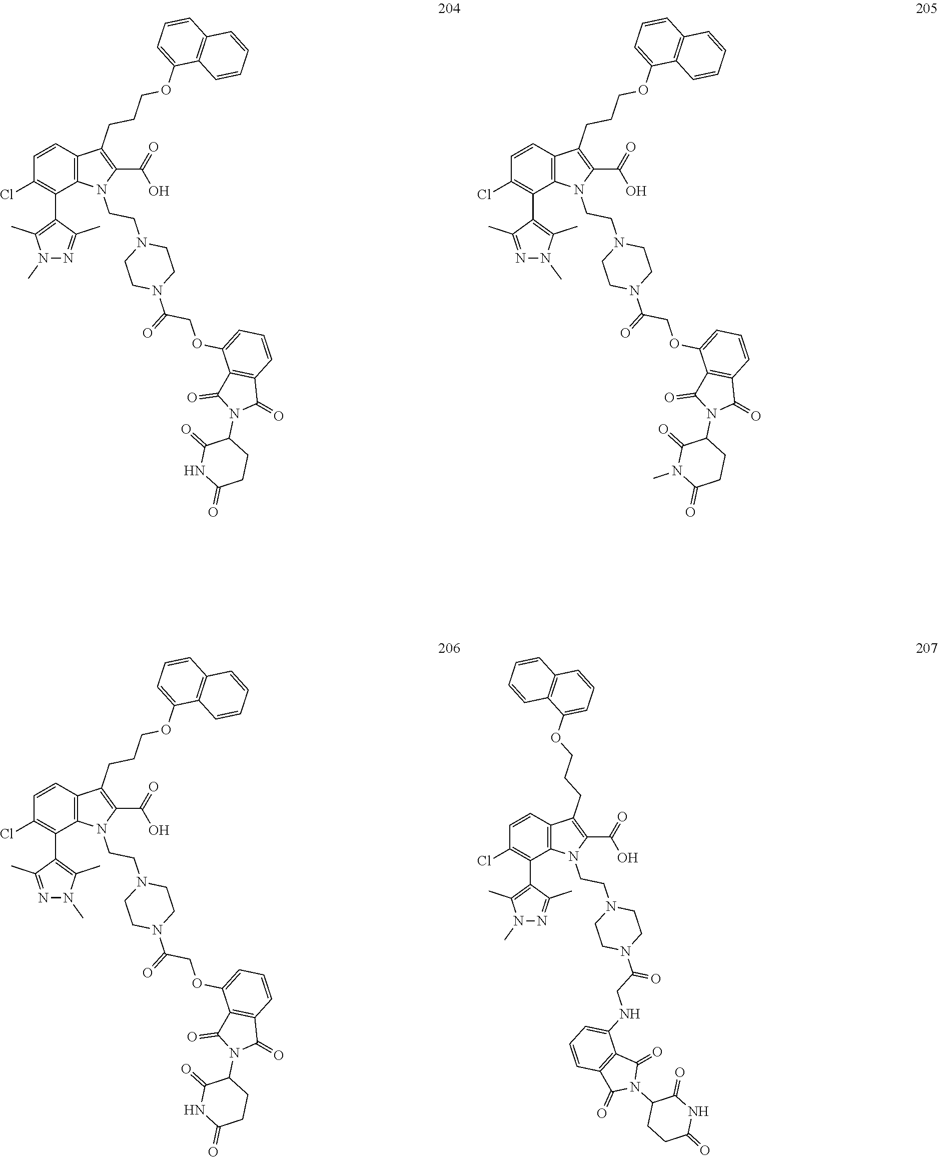 [SMILES: Cc1nn(C)c(C)c1-c1c(Cl)ccc2c(CCCOc3cccc4ccccc34)c(C(=O)O)n(CCN3CCN(C(=O)COc4cccc5c4C(=O)N(C4CCC(=O)NC4=O)C5=O)CC3)c12 ; Cc1nn(C)c(C)c1-c1c(Cl)ccc2c(CCCOc3cccc4ccccc34)c(C(=O)O)n(CCN3CCN(C(=O)COc4cccc5c4C(=O)N(C4CCC(=O)N(C)C4=O)C5=O)CC3)c12 ; Cc1nn(C)c(C)c1-c1c(Cl)ccc2c(CCCOc3cccc4ccccc34)c(C(=O)O)n(CCN3CCN(C(=O)COc4cccc5c4C(=O)N(C4CCC(=O)NC4=O)C5=O)CC3)c12 ; Cc1nn(C)c(C)c1-c1c(Cl)ccc2c(CCCOc3cccc4ccccc34)c(C(=O)O)n(CCN3CCN(C(=O)CNc4cccc5c4C(=O)N(C4CCC(=O)NC4=O)C5=O)CC3)c12]

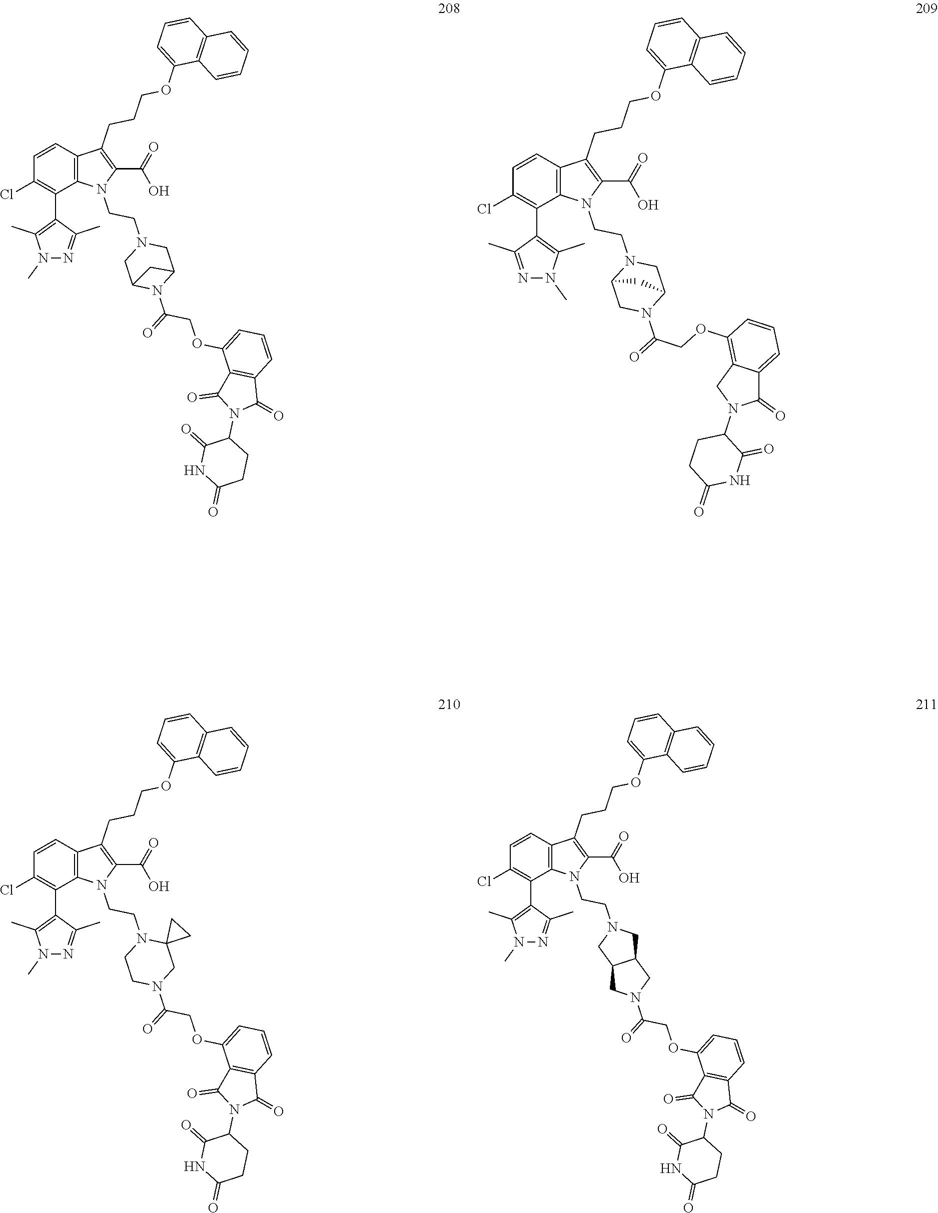 [SMILES: Cc1nn(C)c(C)c1-c1c(Cl)ccc2c(CCCOc3cccc4ccccc34)c(C(=O)O)n(CCN3CC4CC(C3)N4C(=O)COc3cccc4c3C(=O)N(C3CCC(=O)NC3=O)C4=O)c12 ; Cc1nn(C)c(C)c1-c1c(Cl)ccc2c(CCCOc3cccc4ccccc34)c(C(=O)O)n(CCN3C[C@@H]4C[C@H]3CN4C(=O)COc3cccc4c3CN(C3CCC(=O)NC3=O)C4=O)c12 ; Cc1nn(C)c(C)c1-c1c(Cl)ccc2c(CCCOc3cccc4ccccc34)c(C(=O)O)n(CCN3CCN(C(=O)COc4cccc5c4C(=O)N(C4CCC(=O)NC4=O)C5=O)CC34CC4)c12 ; Cc1nn(C)c(C)c1-c1c(Cl)ccc2c(CCCOc3cccc4ccccc34)c(C(=O)O)n(CCN3C[C@@H]4CN(C(=O)COc5cccc6c5C(=O)N(C5CCC(=O)NC5=O)C6=O)C[C@@H]4C3)c12]

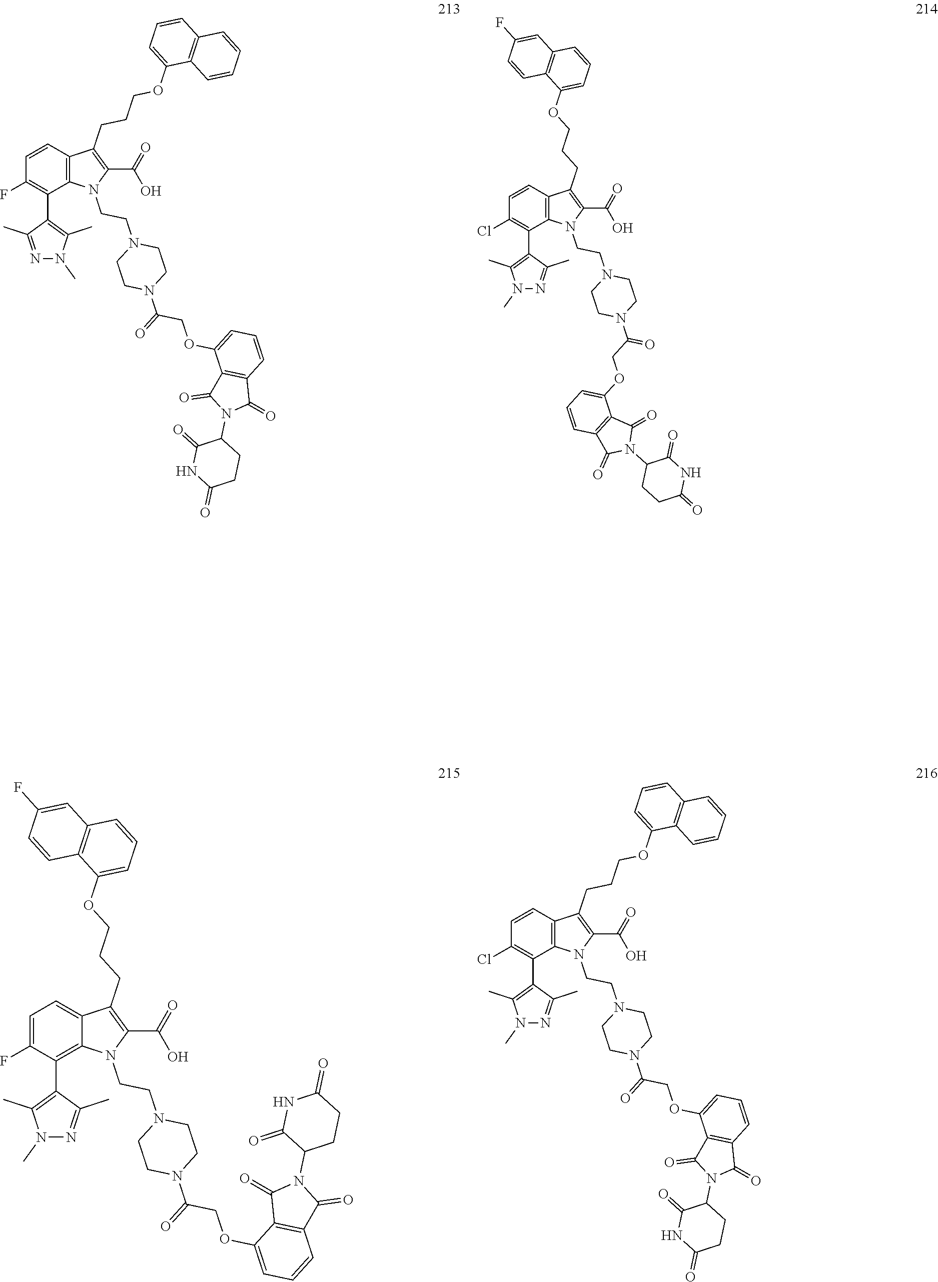 [SMILES: Cc1nn(C)c(C)c1-c1c(F)ccc2c(CCCOc3cccc4ccccc34)c(C(=O)O)n(CCN3CCN(C(=O)COc4cccc5c4C(=O)N(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4cccc5c4C(=O)N(C4CCC(=O)NC4=O)C5=O)CC3)c12 ; Cc1nn(C)c(C)c1-c1c(F)ccc2c(CCCOc3cccc4cc(F)ccc34)c(C(=O)O)n(CCN3CCN(C(=O)COc4cccc5c4C(=O)N(C4CCC(=O)NC4=O)C5=O)CC3)c12 ; Cc1nn(C)c(C)c1-c1c(Cl)ccc2c(CCCOc3cccc4ccccc34)c(C(=O)O)n(CCN3CCN(C(=O)COc4cccc5c4C(=O)N(C4CCC(=O)NC4=O)C5=O)CC3)c12]

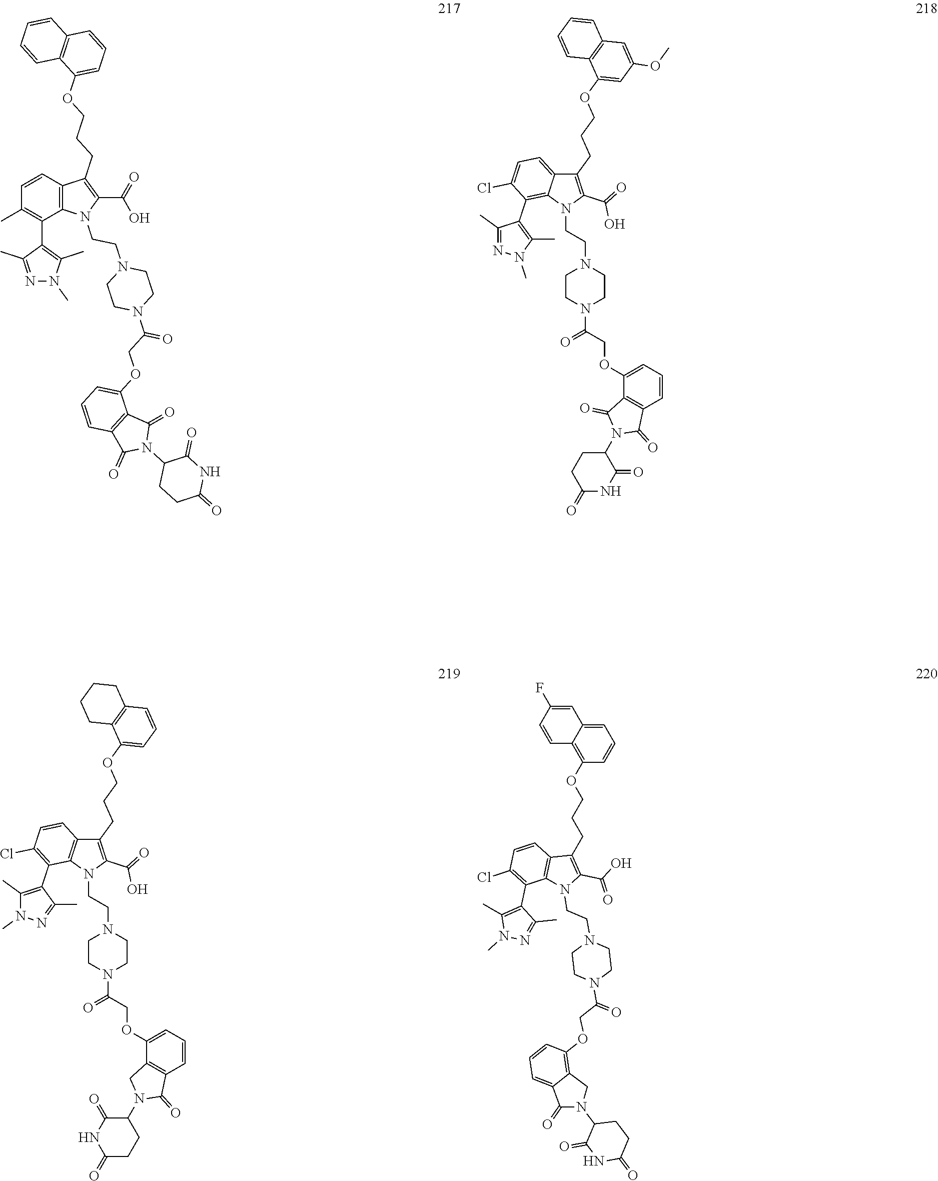

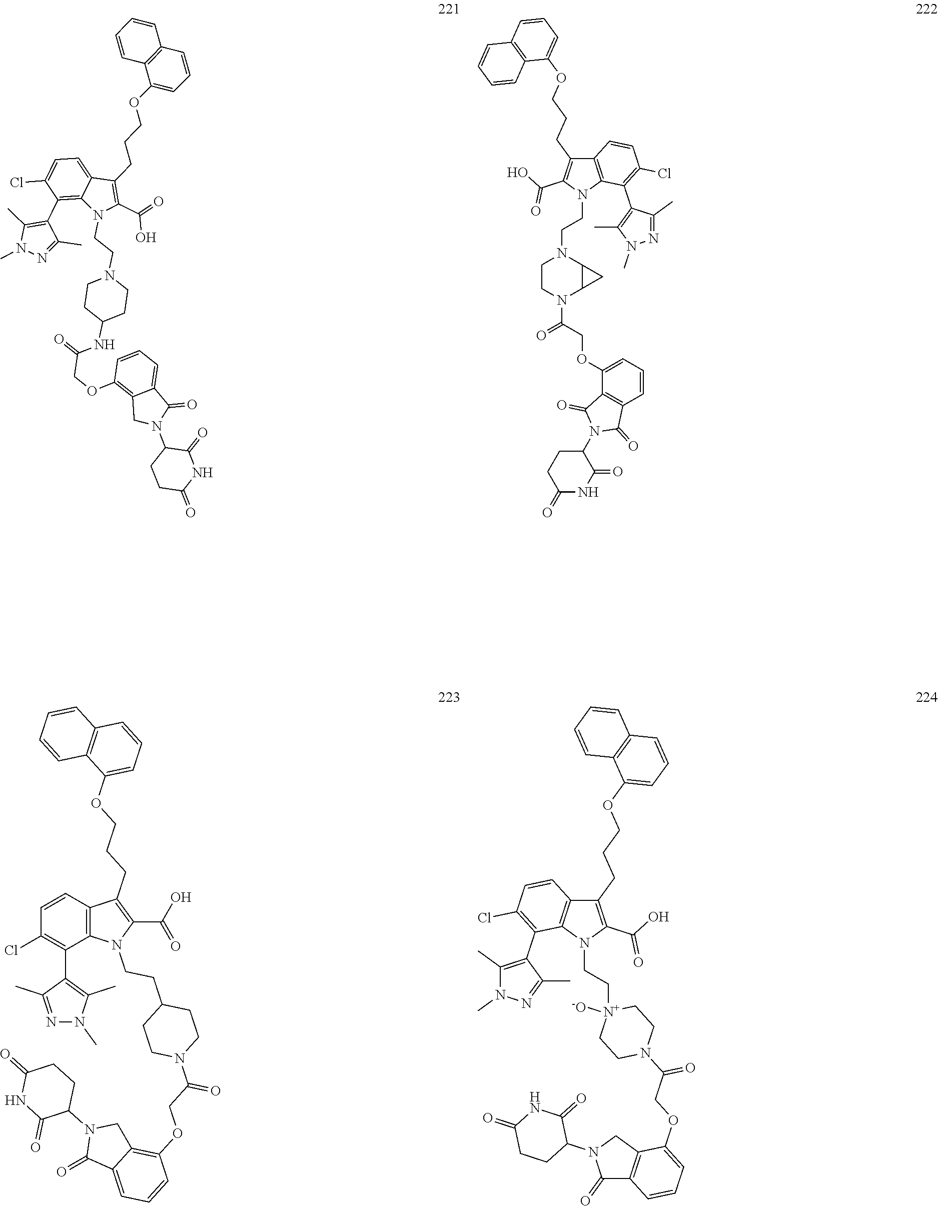

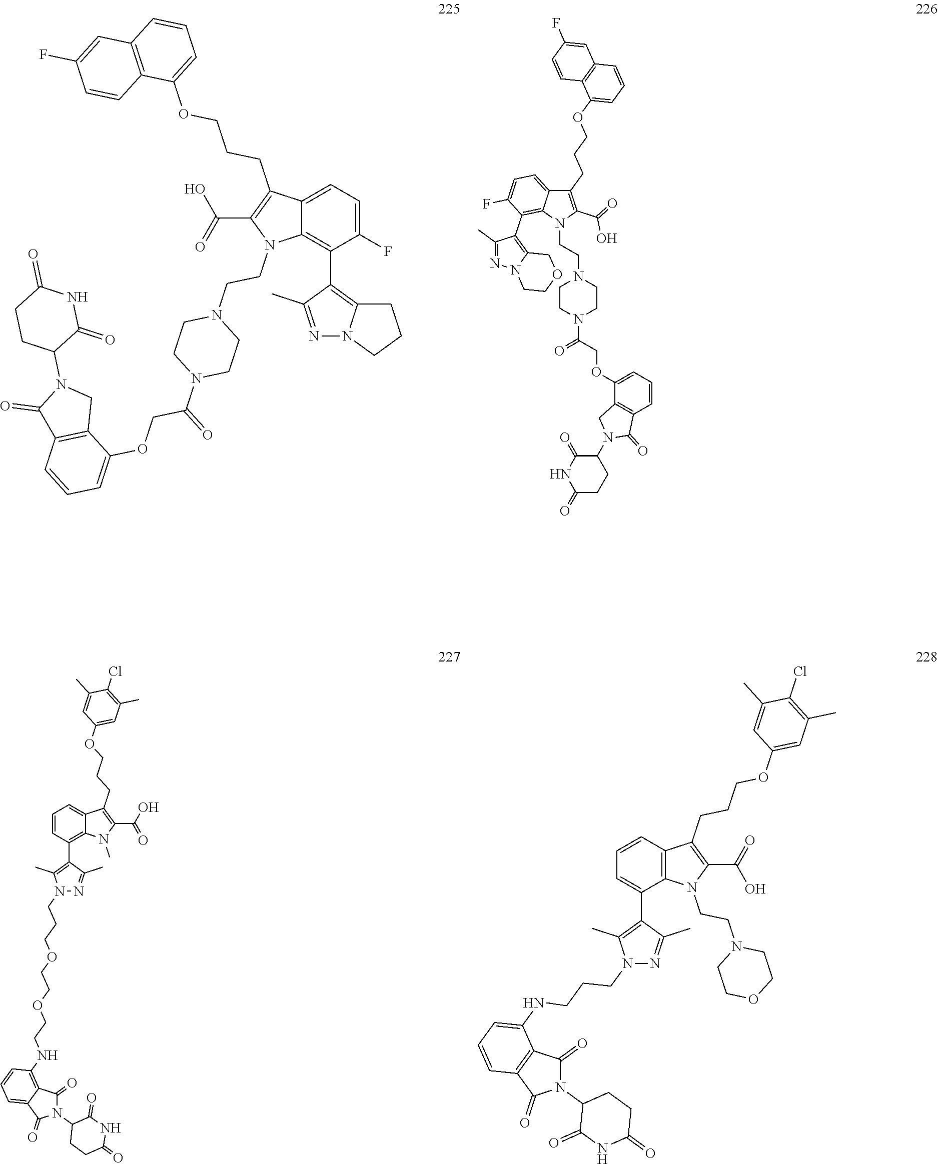 [SMILES: Cc1nn2c(c1-c1c(F)ccc3c(CCCOc4cccc5cc(F)ccc45)c(C(=O)O)n(CCN4CCN(C(=O)COc5cccc6c5CN(C5CCC(=O)NC5=O)C6=O)CC4)c13)CCC2 ; Cc1nn2c(c1-c1c(F)ccc3c(CCCOc4cccc5cc(F)ccc45)c(C(=O)O)n(CCN4CCN(C(=O)COc5cccc6c5CN(C5CCC(=O)NC5=O)C6=O)CC4)c13)COCC2 ; Cc1cc(OCCCc2c(C(=O)O)n(C)c3c(-c4c(C)nn(CCCOCCOCCNc5cccc6c5C(=O)N(C5CCC(=O)NC5=O)C6=O)c4C)cccc23)cc(C)c1Cl ; Cc1cc(OCCCc2c(C(=O)O)n(CCN3CCOCC3)c3c(-c4c(C)nn(CCCNc5cccc6c5C(=O)N(C5CCC(=O)NC5=O)C6=O)c4C)cccc23)cc(C)c1Cl]

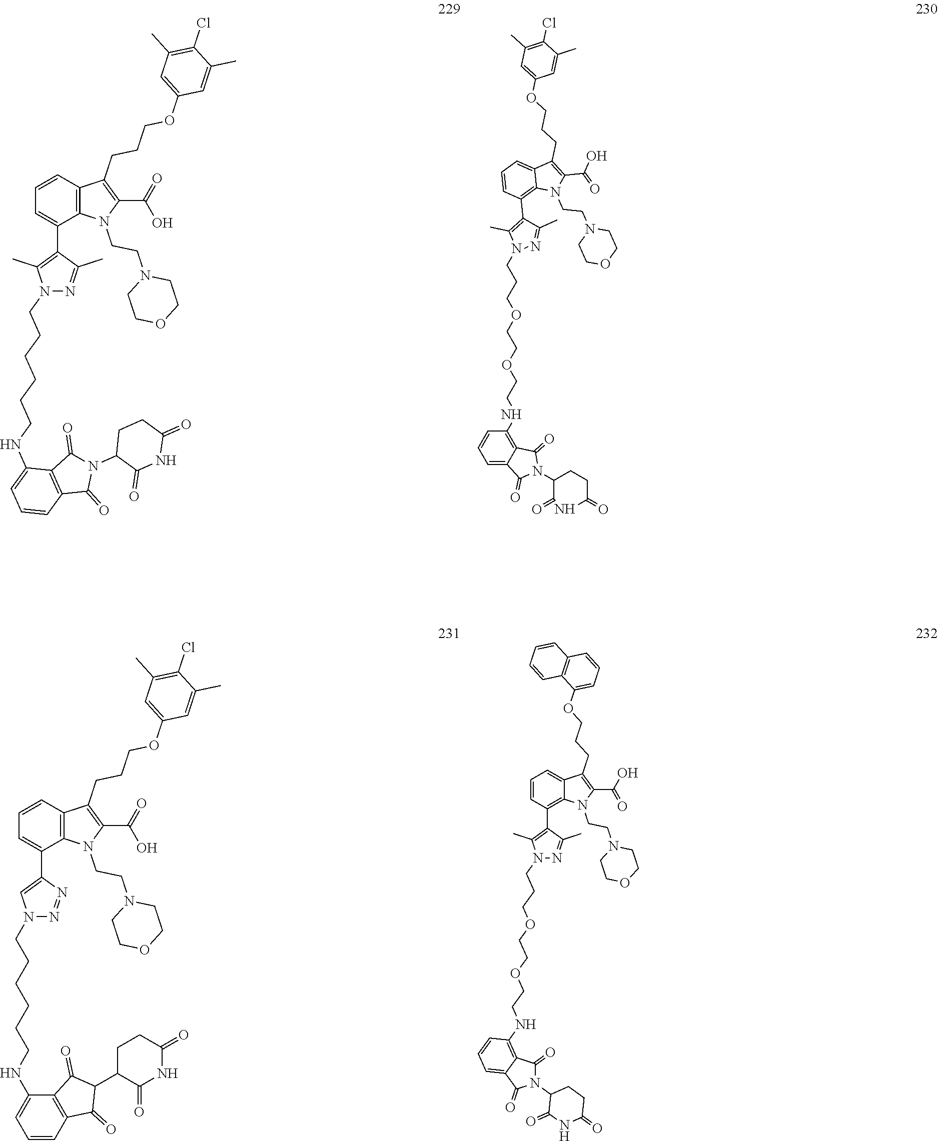 [SMILES: Cc1cc(OCCCc2c(C(=O)O)n(CCN3CCOCC3)c3c(-c4c(C)nn(CCCCCCNc5cccc6c5C(=O)N(C5CCC(=O)NC5=O)C6=O)c4C)cccc23)cc(C)c1Cl ; Cc1cc(OCCCc2c(C(=O)O)n(CCN3CCOCC3)c3c(-c4c(C)nn(CCCOCCOCCNc5cccc6c5C(=O)N(C5CCC(=O)NC5=O)C6=O)c4C)cccc23)cc(C)c1Cl ; Cc1cc(OCCCc2c(C(=O)O)n(CCN3CCOCC3)c3c(-c4cn(CCCCCCNc5cccc6c5C(=O)C(C5CCC(=O)NC5=O)C6=O)nn4)cccc23)cc(C)c1Cl ; Cc1nn(CCCOCCOCCNc2cccc3c2C(=O)N(C2CCC(=O)NC2=O)C3=O)c(C)c1-c1cccc2c(CCCOc3cccc4ccccc34)c(C(=O)O)n(CCN3CCOCC3)c12]

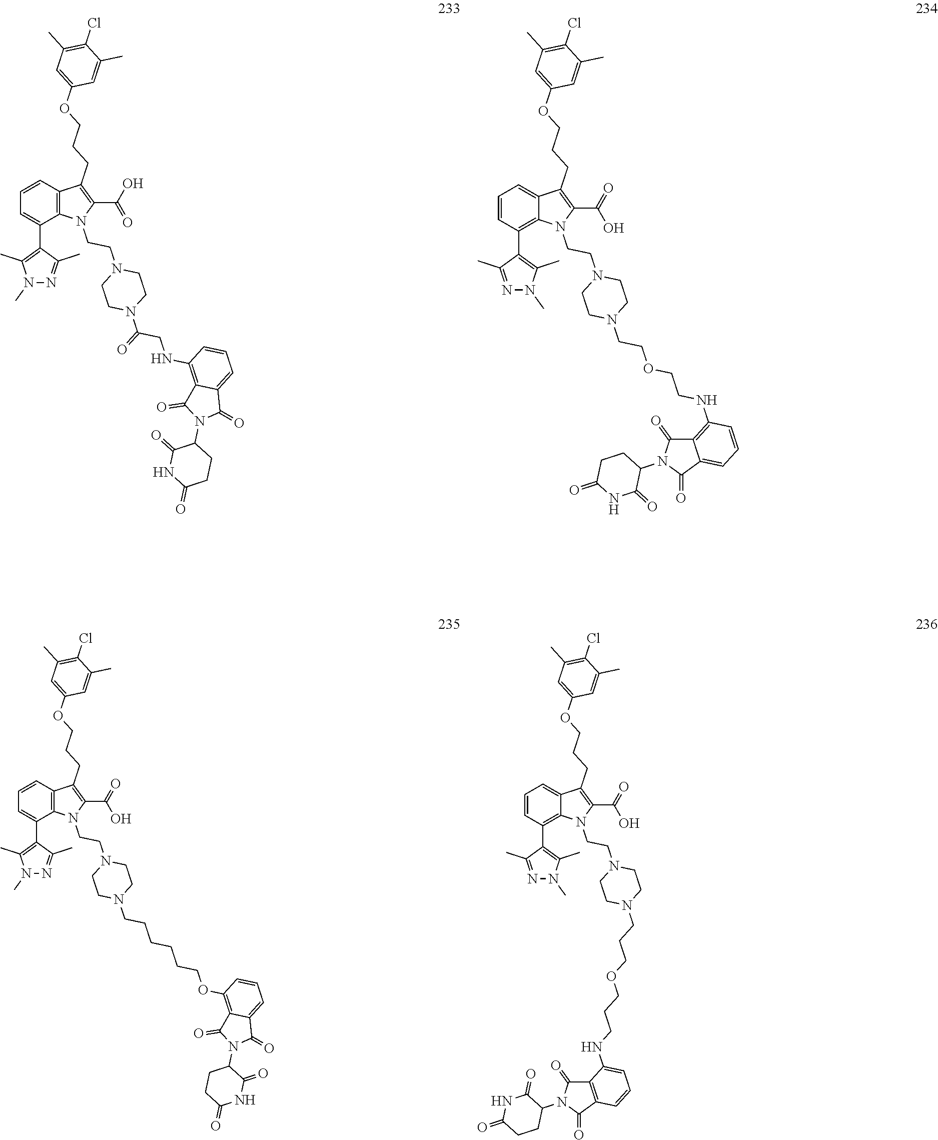 [SMILES: Cc1cc(OCCCc2c(C(=O)O)n(CCN3CCN(C(=O)CNc4cccc5c4C(=O)N(C4CCC(=O)NC4=O)C5=O)CC3)c3c(-c4c(C)nn(C)c4C)cccc23)cc(C)c1Cl ; Cc1cc(OCCCc2c(C(=O)O)n(CCN3CCN(CCOCCNc4cccc5c4C(=O)N(C4CCC(=O)NC4=O)C5=O)CC3)c3c(-c4c(C)nn(C)c4C)cccc23)cc(C)c1Cl ; Cc1cc(OCCCc2c(C(=O)O)n(CCN3CCN(CCCCCCOc4cccc5c4C(=O)N(C4CCC(=O)NC4=O)C5=O)CC3)c3c(-c4c(C)nn(C)c4C)cccc23)cc(C)c1Cl ; Cc1cc(OCCCc2c(C(=O)O)n(CCN3CCN(CCCOCCCNc4cccc5c4C(=O)N(C4CCC(=O)NC4=O)C5=O)CC3)c3c(-c4c(C)nn(C)c4C)cccc23)cc(C)c1Cl]

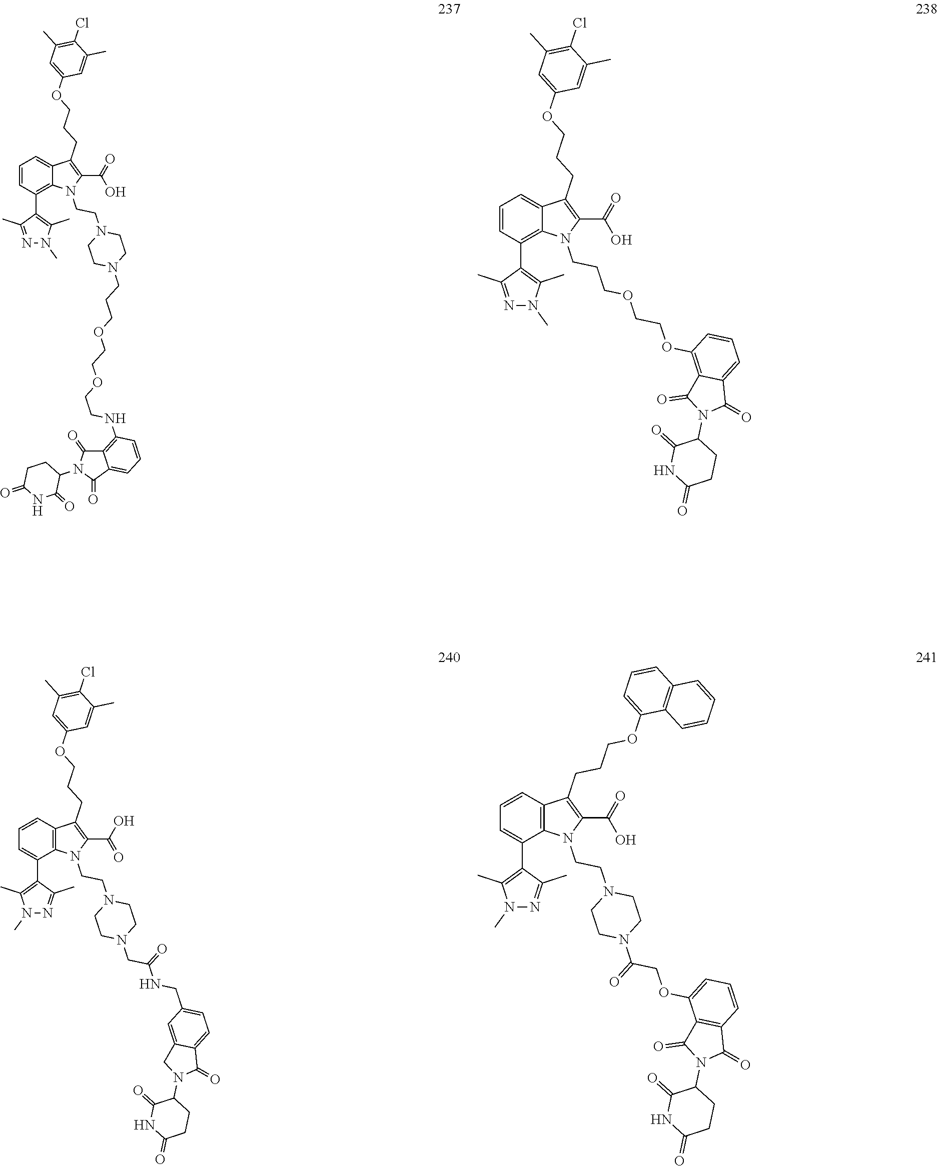 [SMILES: Cc1cc(OCCCc2c(C(=O)O)n(CCN3CCN(CCCOCCOCCNc4cccc5c4C(=O)N(C4CCC(=O)NC4=O)C5=O)CC3)c3c(-c4c(C)nn(C)c4C)cccc23)cc(C)c1Cl ; Cc1cc(OCCCc2c(C(=O)O)n(CCCOCCOc3cccc4c3C(=O)N(C3CCC(=O)NC3=O)C4=O)c3c(-c4c(C)nn(C)c4C)cccc23)cc(C)c1Cl ; Cc1cc(OCCCc2c(C(=O)O)n(CCN3CCN(CC(=O)NCc4ccc5c(c4)CN(C4CCC(=O)NC4=O)C5=O)CC3)c3c(-c4c(C)nn(C)c4C)cccc23)cc(C)c1Cl ; Cc1nn(C)c(C)c1-c1cccc2c(CCCOc3cccc4ccccc34)c(C(=O)O)n(CCN3CCN(C(=O)COc4cccc5c4C(=O)N(C4CCC(=O)NC4=O)C5=O)CC3)c12]

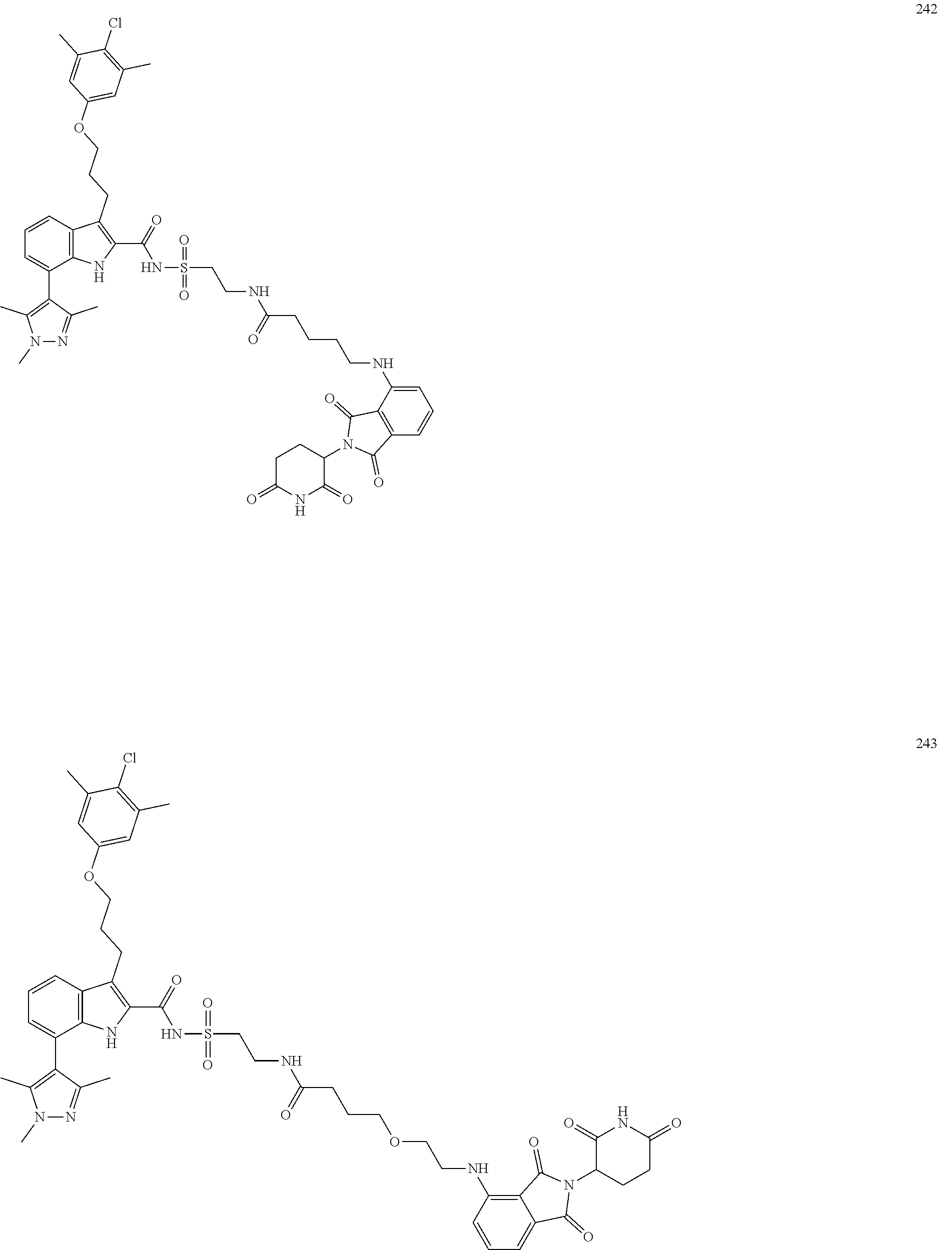 [SMILES: Cc1cc(OCCCc2c(C(=O)NS(=O)(=O)CCNC(=O)CCCCNc3cccc4c3C(=O)N(C3CCC(=O)NC3=O)C4=O)[nH]c3c(-c4c(C)nn(C)c4C)cccc23)cc(C)c1Cl ; Cc1cc(OCCCc2c(C(=O)NS(=O)(=O)CCNC(=O)CCCOCCNc3cccc4c3C(=O)N(C3CCC(=O)NC3=O)C4=O)[nH]c3c(-c4c(C)nn(C)c4C)cccc23)cc(C)c1Cl]

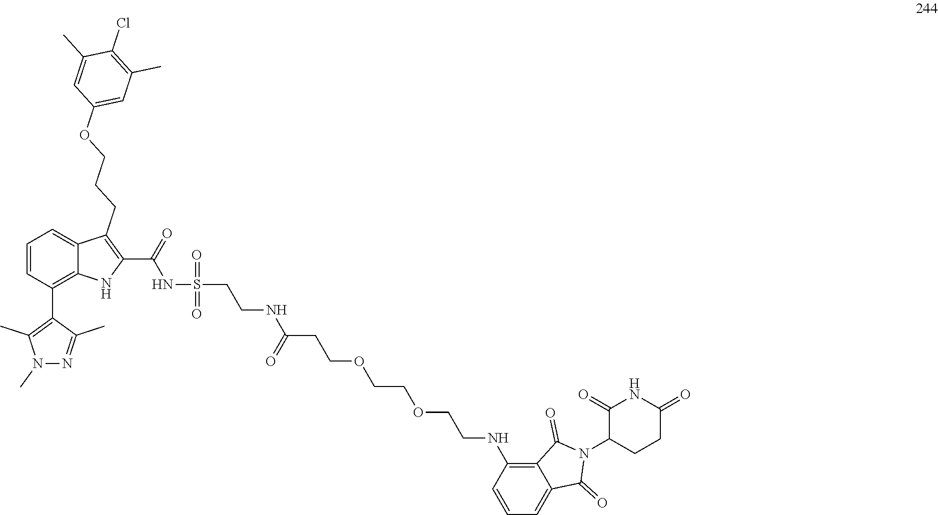 [SMILES: Cc1cc(OCCCc2c(C(=O)NS(=O)(=O)CCNC(=O)CCOCCOCCNc3cccc4c3C(=O)N(C3CCC(=O)NC3=O)C4=O)[nH]c3c(-c4c(C)nn(C)c4C)cccc23)cc(C)c1Cl]

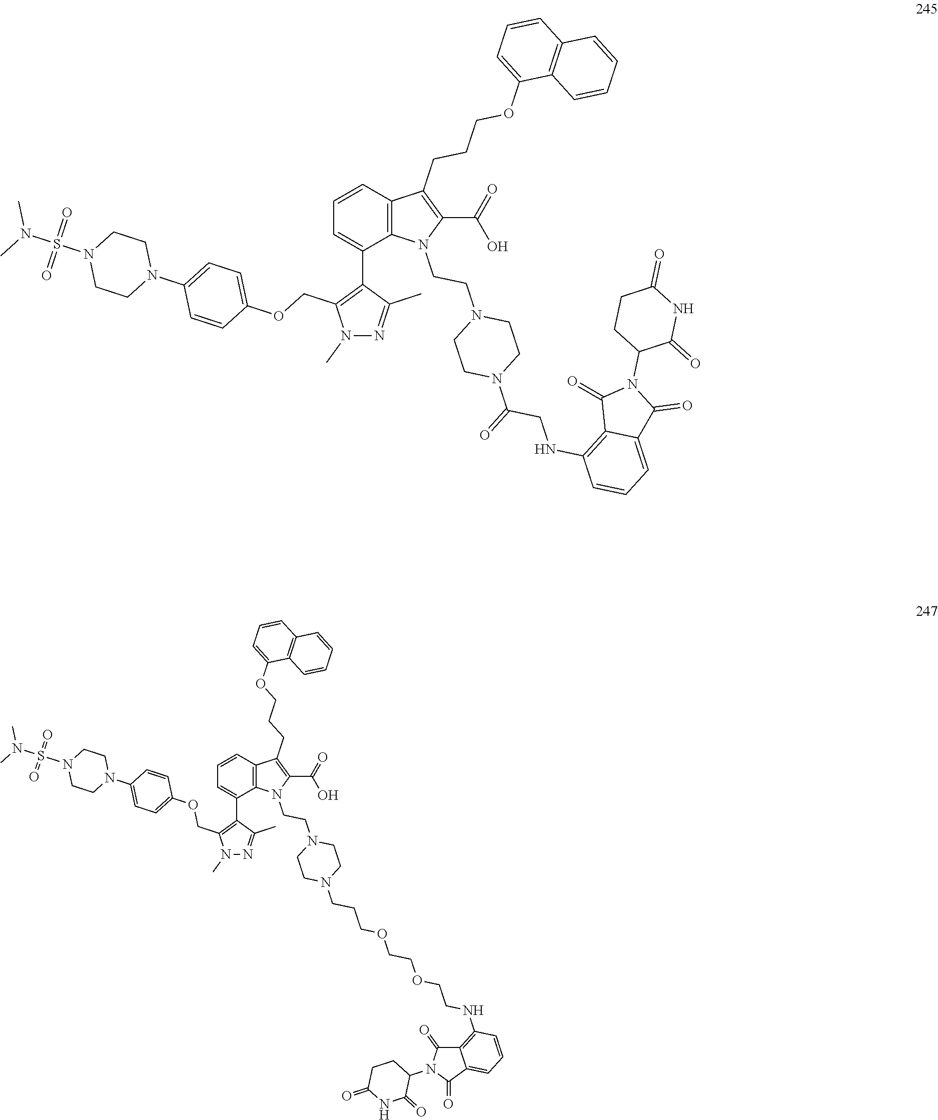 [SMILES: Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(C(=O)CNc4cccc5c4C(=O)N(C4CCC(=O)NC4=O)C5=O)CC3)c12 ; Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(CCCOCCOCCNc4cccc5c4C(=O)N(C4CCC(=O)NC4=O)C5=O)CC3)c12]

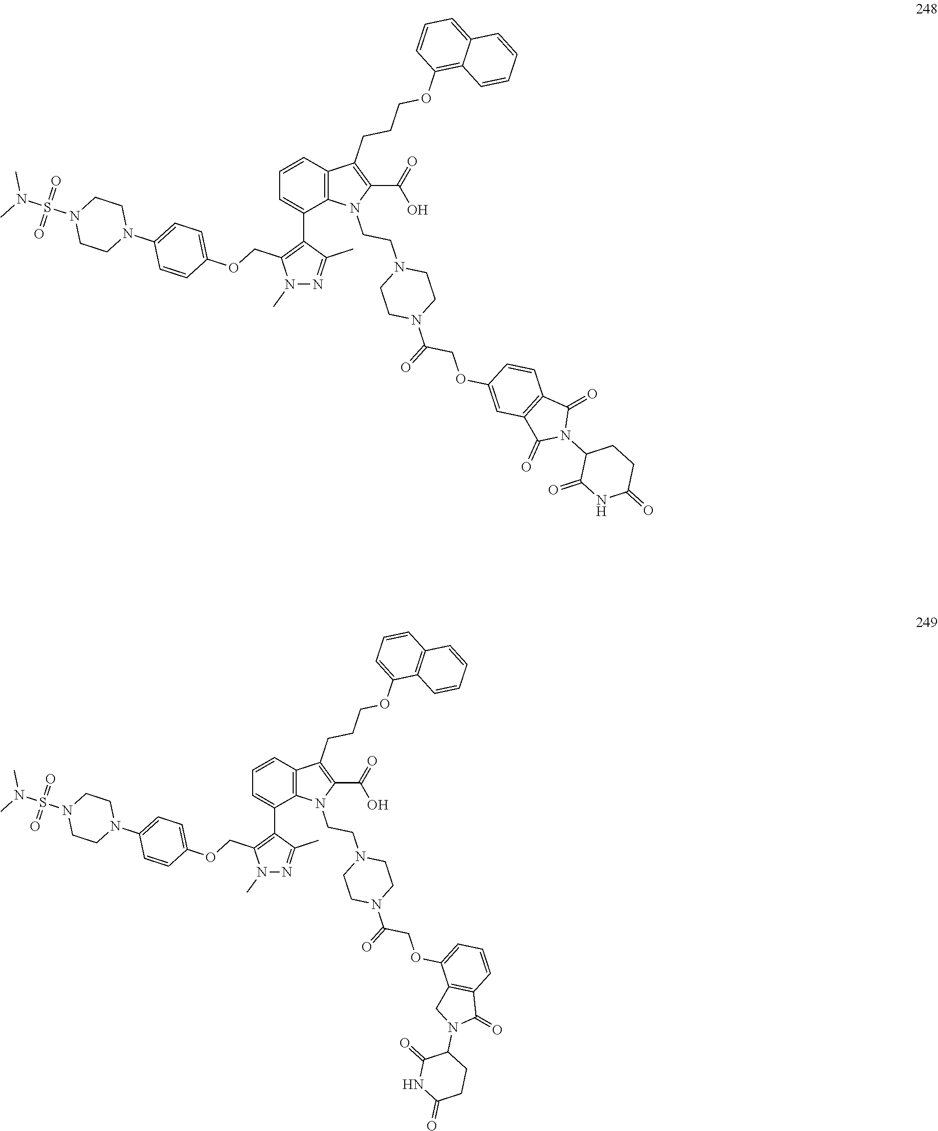 [SMILES: Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(C(=O)COc4ccc5c(c4)C(=O)N(C4CCC(=O)NC4=O)C5=O)CC3)c12 ; Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(C(=O)COc4cccc5c4CN(C4CCC(=O)NC4=O)C5=O)CC3)c12]

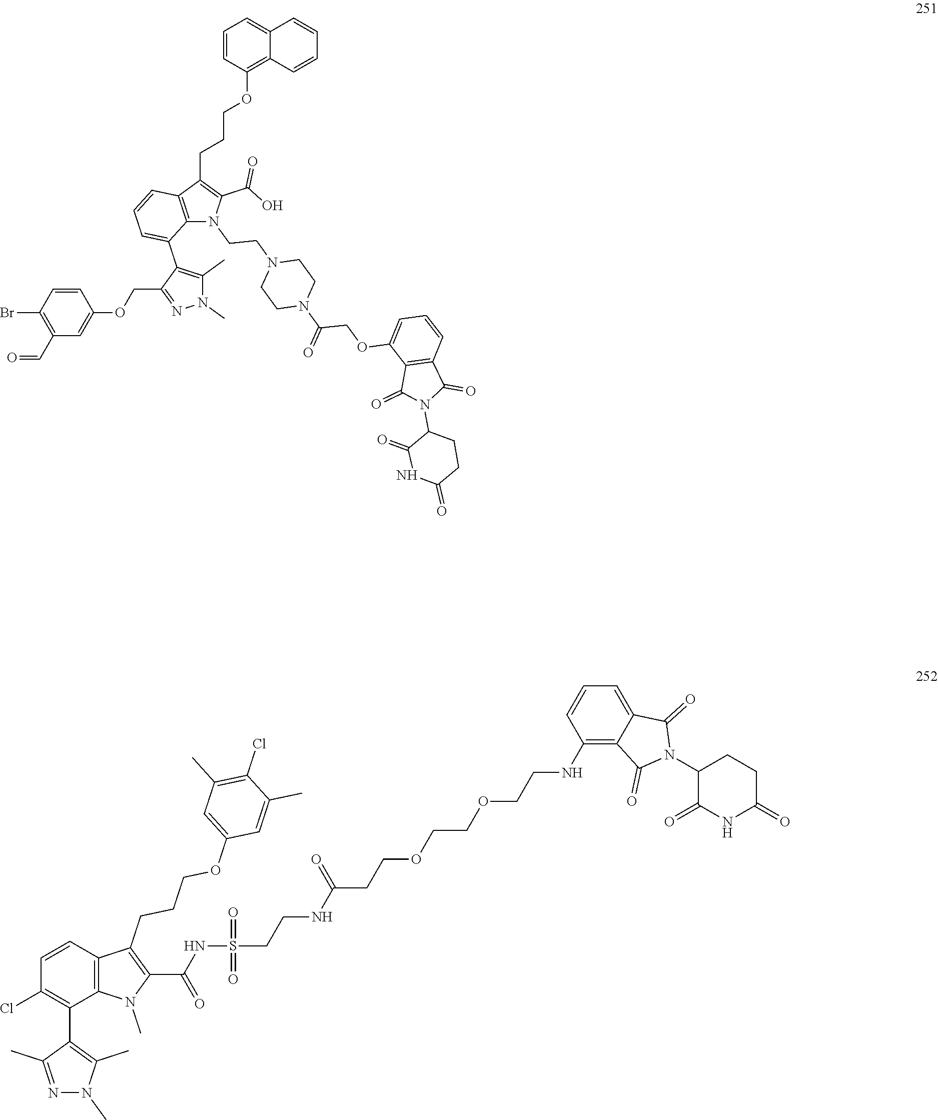 [SMILES: Cc1c(-c2cccc3c(CCCOc4cccc5ccccc45)c(C(=O)O)n(CCN4CCN(C(=O)COc5cccc6c5C(=O)N(C5CCC(=O)NC5=O)C6=O)CC4)c23)c(COc2ccc(Br)c(C=O)c2)nn1C ; Cc1cc(OCCCc2c(C(=O)NS(=O)(=O)CCNC(=O)CCOCCOCCNc3cccc4c3C(=O)N(C3CCC(=O)NC3=O)C4=O)n(C)c3c(-c4c(C)nn(C)c4C)c(Cl)ccc23)cc(C)c1Cl]

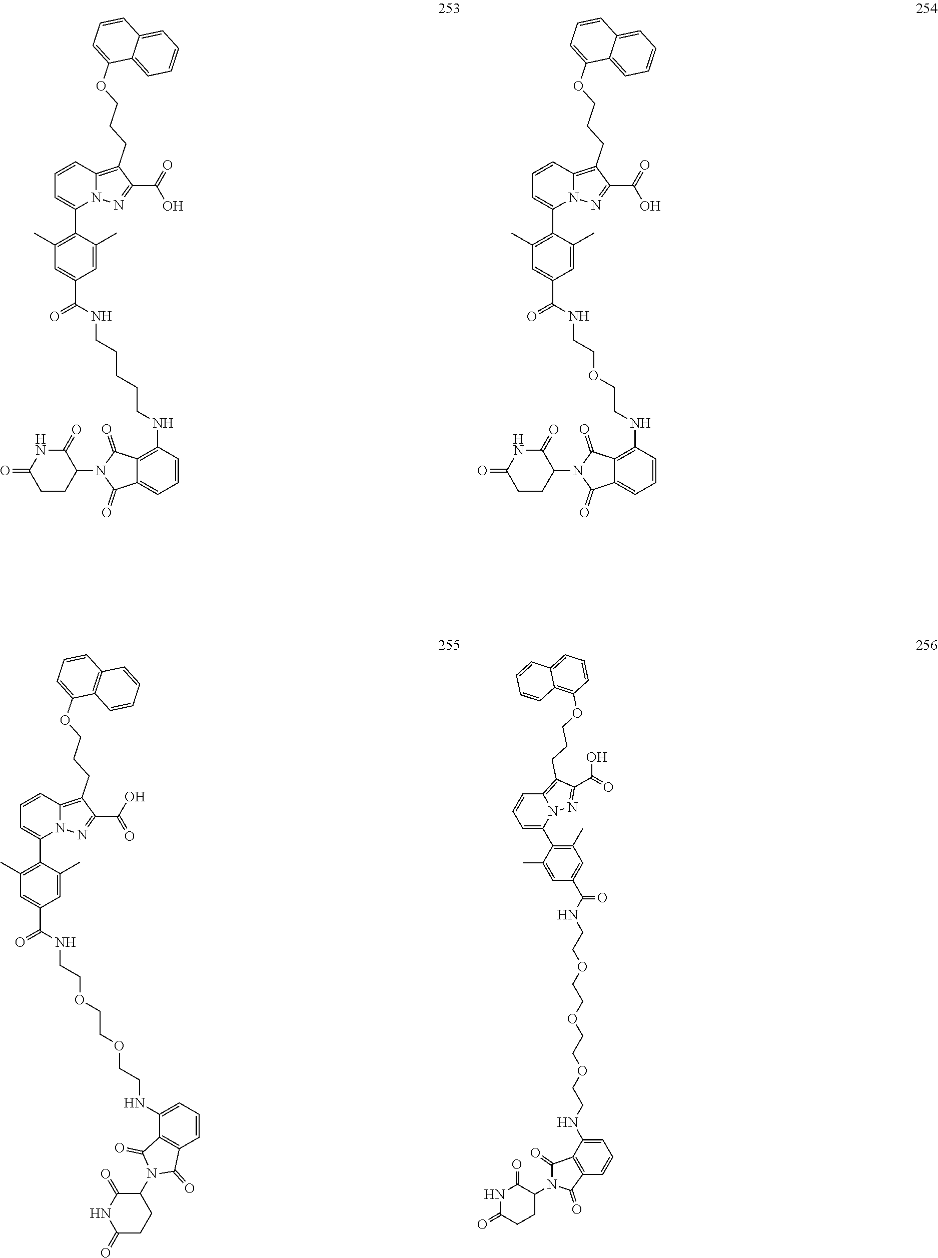 [SMILES: Cc1cc(C(=O)NCCCCCNc2cccc3c2C(=O)N(C2CCC(=O)NC2=O)C3=O)cc(C)c1-c1cccc2c(CCCOc3cccc4ccccc34)c(C(=O)O)nn12 ; Cc1cc(C(=O)NCCOCCNc2cccc3c2C(=O)N(C2CCC(=O)NC2=O)C3=O)cc(C)c1-c1cccc2c(CCCOc3cccc4ccccc34)c(C(=O)O)nn12 ; Cc1cc(C(=O)NCCOCCOCCNc2cccc3c2C(=O)N(C2CCC(=O)NC2=O)C3=O)cc(C)c1-c1cccc2c(CCCOc3cccc4ccccc34)c(C(=O)O)nn12 ; Cc1cc(C(=O)NCCOCCOCCOCCNc2cccc3c2C(=O)N(C2CCC(=O)NC2=O)C3=O)cc(C)c1-c1cccc2c(CCCOc3cccc4ccccc34)c(C(=O)O)nn12]

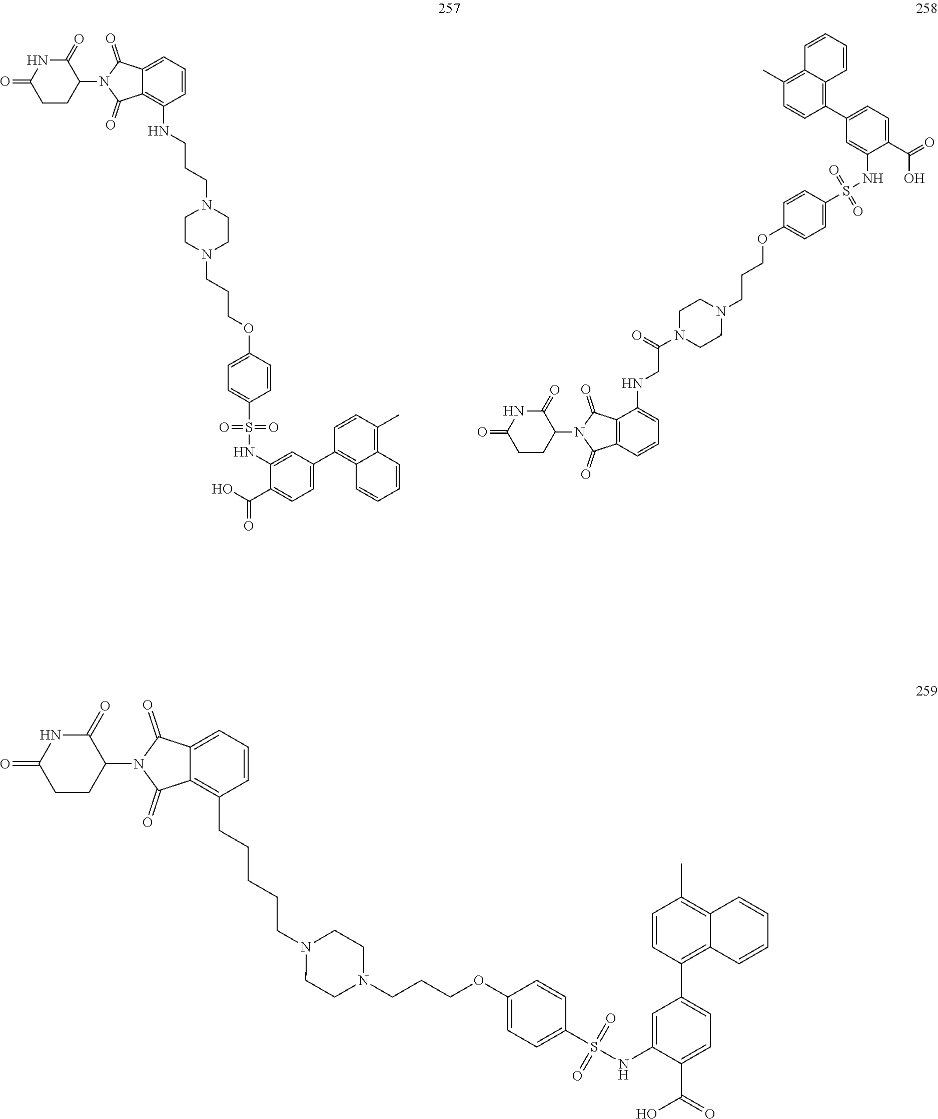 [SMILES: Cc1ccc(-c2ccc(C(=O)O)c(NS(=O)(=O)c3ccc(OCCCN4CCN(CCCNc5cccc6c5C(=O)N(C5CCC(=O)NC5=O)C6=O)CC4)cc3)c2)c2ccccc12 ; Cc1ccc(-c2ccc(C(=O)O)c(NS(=O)(=O)c3ccc(OCCCN4CCN(C(=O)CNc5cccc6c5C(=O)N(C5CCC(=O)NC5=O)C6=O)CC4)cc3)c2)c2ccccc12 ; Cc1ccc(-c2ccc(C(=O)O)c(NS(=O)(=O)c3ccc(OCCCN4CCN(CCCCCc5cccc6c5C(=O)N(C5CCC(=O)NC5=O)C6=O)CC4)cc3)c2)c2ccccc12]

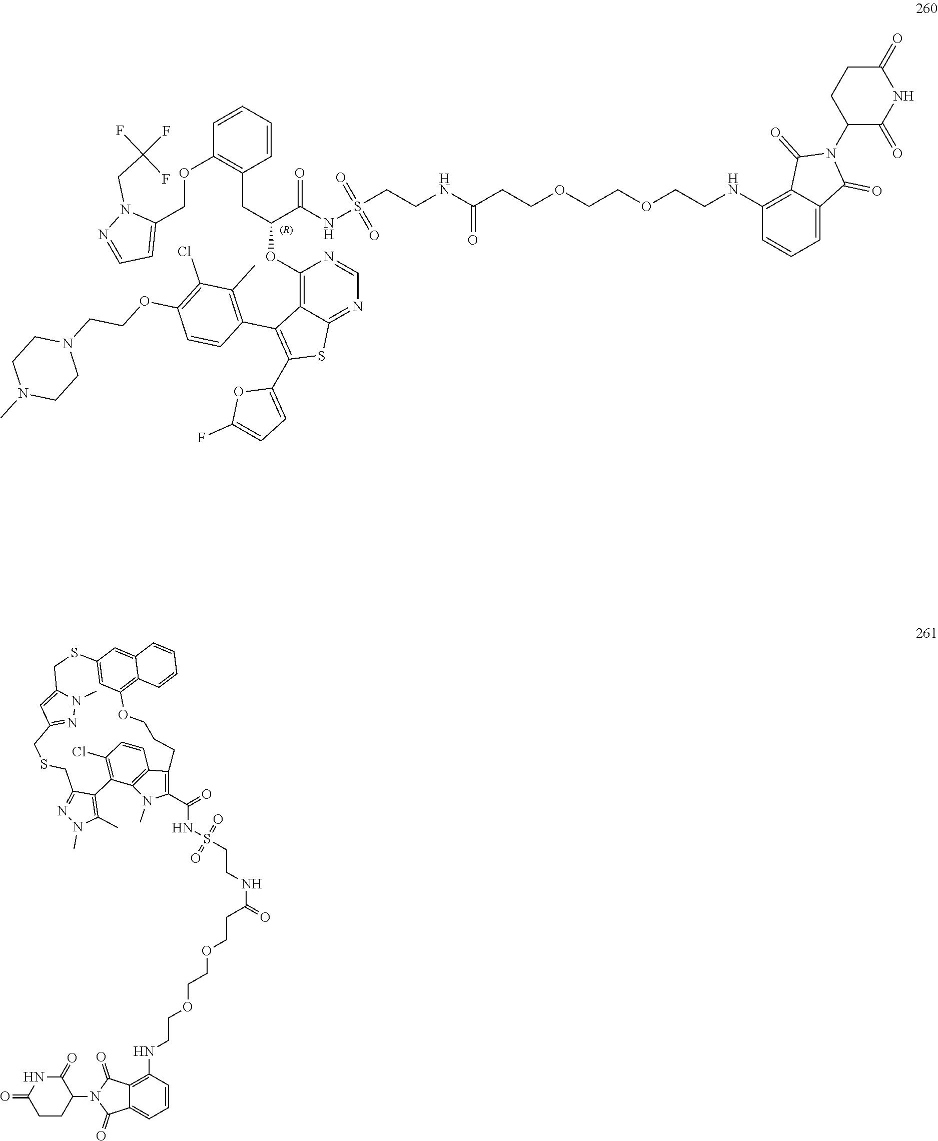

In some embodiments, the compound is selected from:

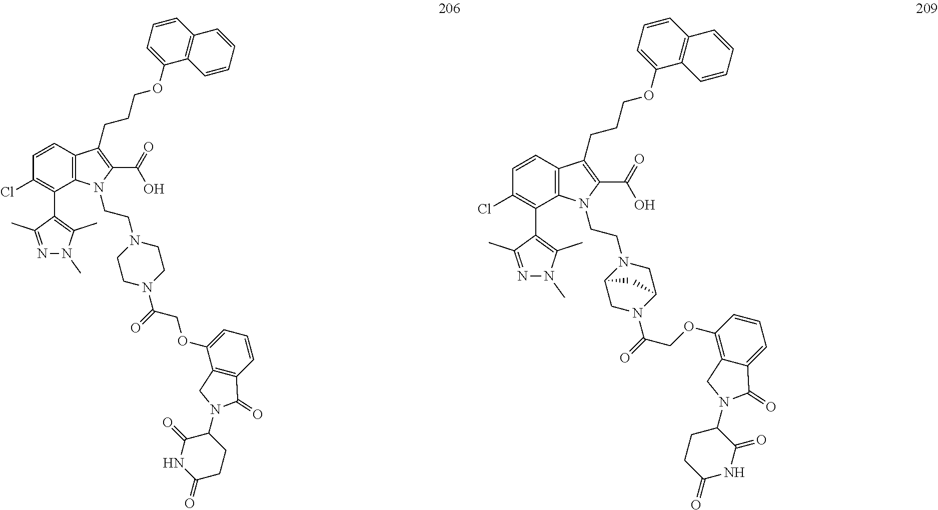 [SMILES: Cc1nn(C)c(C)c1-c1c(Cl)ccc2c(CCCOc3cccc4ccccc34)c(C(=O)O)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4ccccc34)c(C(=O)O)n(CCN3C[C@@H]4C[C@H]3CN4C(=O)COc3cccc4c3CN(C3CCC(=O)NC3=O)C4=O)c12]

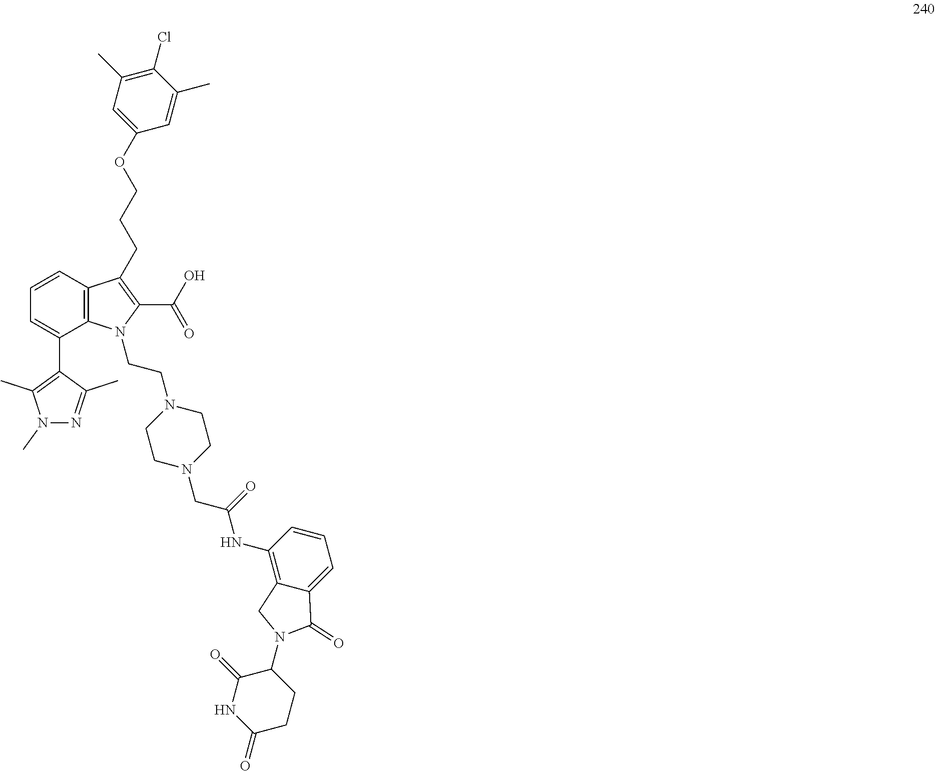 [SMILES: Cc1cc(OCCCc2c(C(=O)O)n(CCN3CCN(CC(=O)Nc4cccc5c4CN(C4CCC(=O)NC4=O)C5=O)CC3)c3c(-c4c(C)nn(C)c4C)cccc23)cc(C)c1Cl]

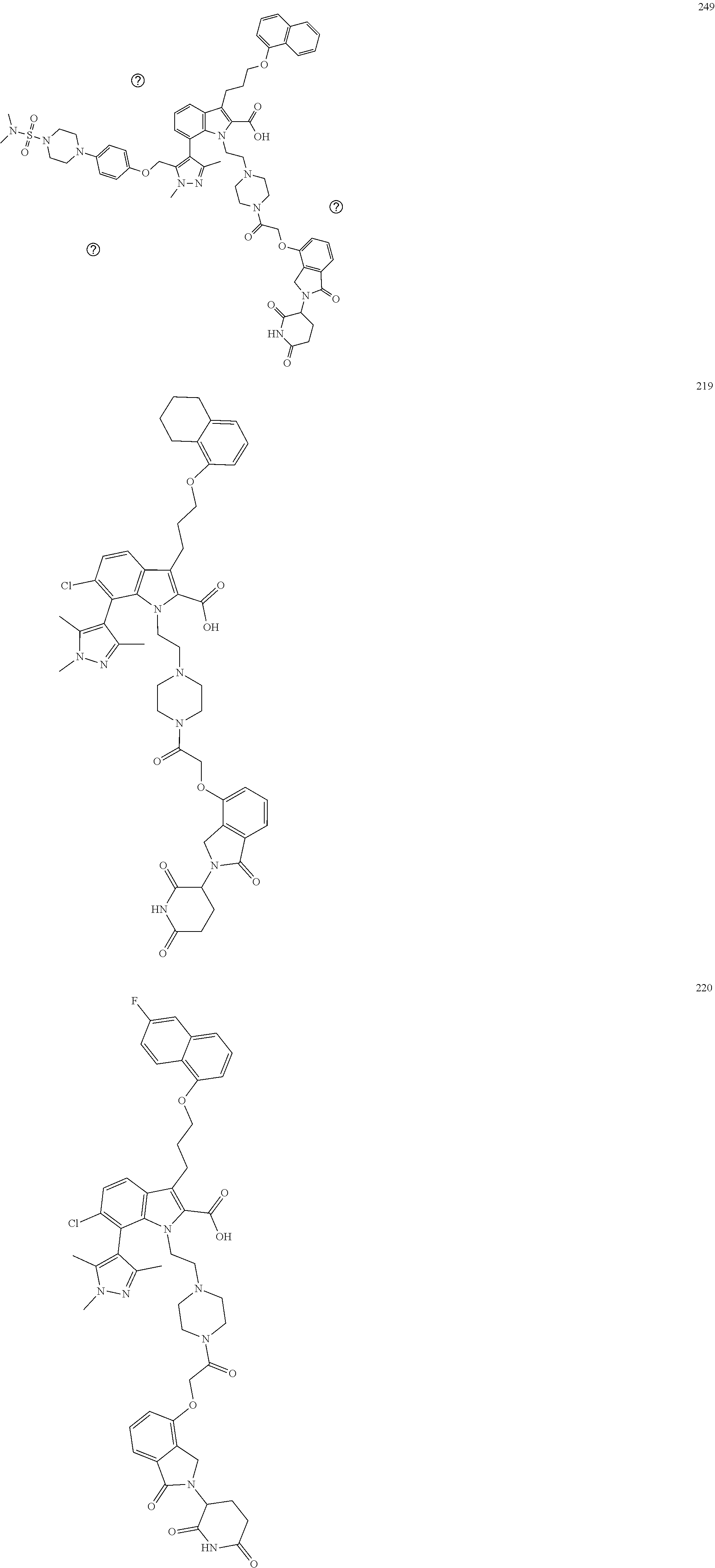 [SMILES: Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4c3CCCC4)c(C(=O)O)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4cccc5c4CN(C4CCC(=O)NC4=O)C5=O)CC3)c12]

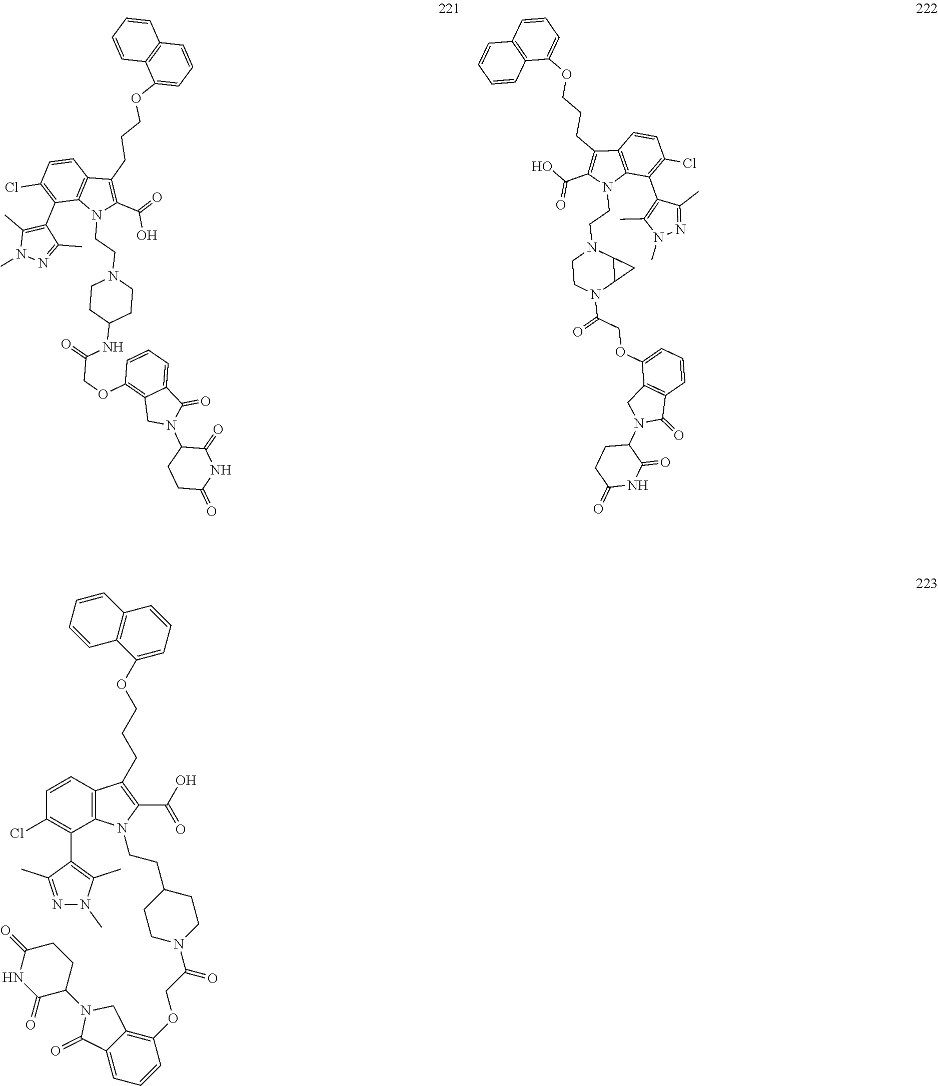 [SMILES: Cc1nn(C)c(C)c1-c1c(Cl)ccc2c(CCCOc3cccc4ccccc34)c(C(=O)O)n(CCN3CCC(NC(=O)COc4cccc5c4CN(C4CCC(=O)NC4=O)C5=O)CC3)c12 ; Cc1nn(C)c(C)c1-c1c(Cl)ccc2c(CCCOc3cccc4ccccc34)c(C(=O)O)n(CCN3CCN(C(=O)COc4cccc5c4CN(C4CCC(=O)NC4=O)C5=O)C4CC43)c12 ; Cc1nn(C)c(C)c1-c1c(Cl)ccc2c(CCCOc3cccc4ccccc34)c(C(=O)O)n(CCC3CCN(C(=O)COc4cccc5c4CN(C4CCC(=O)NC4=O)C5=O)CC3)c12]

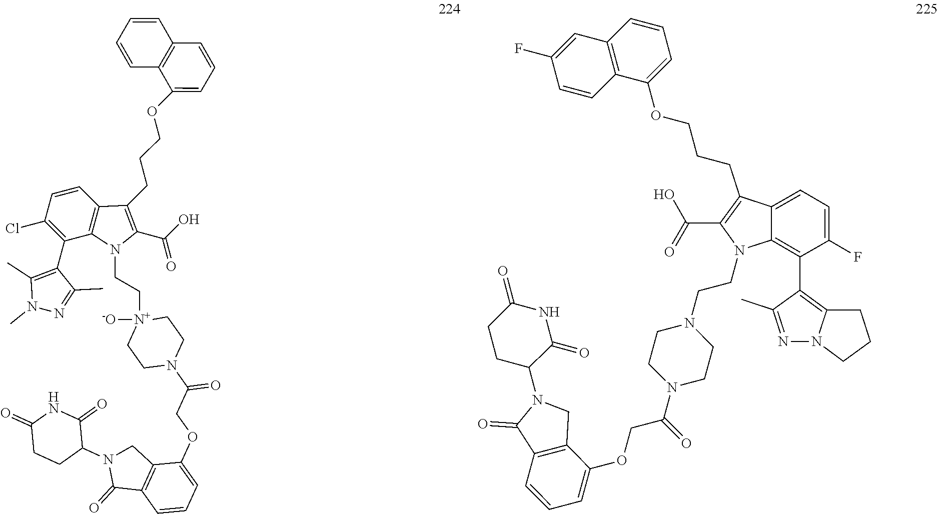

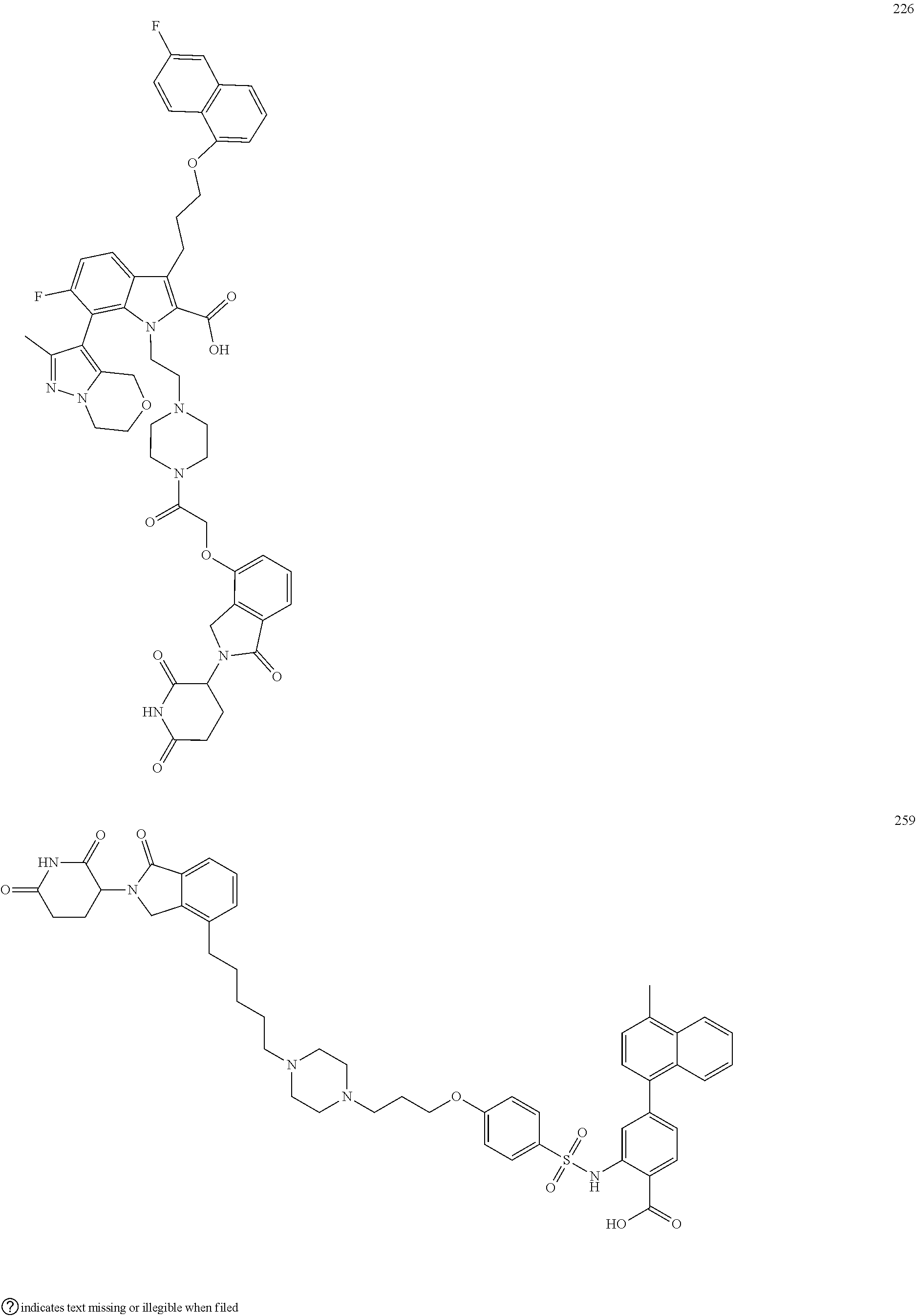 [SMILES: Cc1nn2c(c1-c1c(F)ccc3c(CCCOc4cccc5cc(F)ccc45)c(C(=O)O)n(CCN4CCN(C(=O)COc5cccc6c5CN(C5CCC(=O)NC5=O)C6=O)CC4)c13)COCC2 ; Cc1ccc(-c2ccc(C(=O)O)c(NS(=O)(=O)c3ccc(OCCCN4CCN(CCCCCc5cccc6c5CN(C5CCC(=O)NC5=O)C6=O)CC4)cc3)c2)c2ccccc12]

In some embodiments, the compound is selected from:

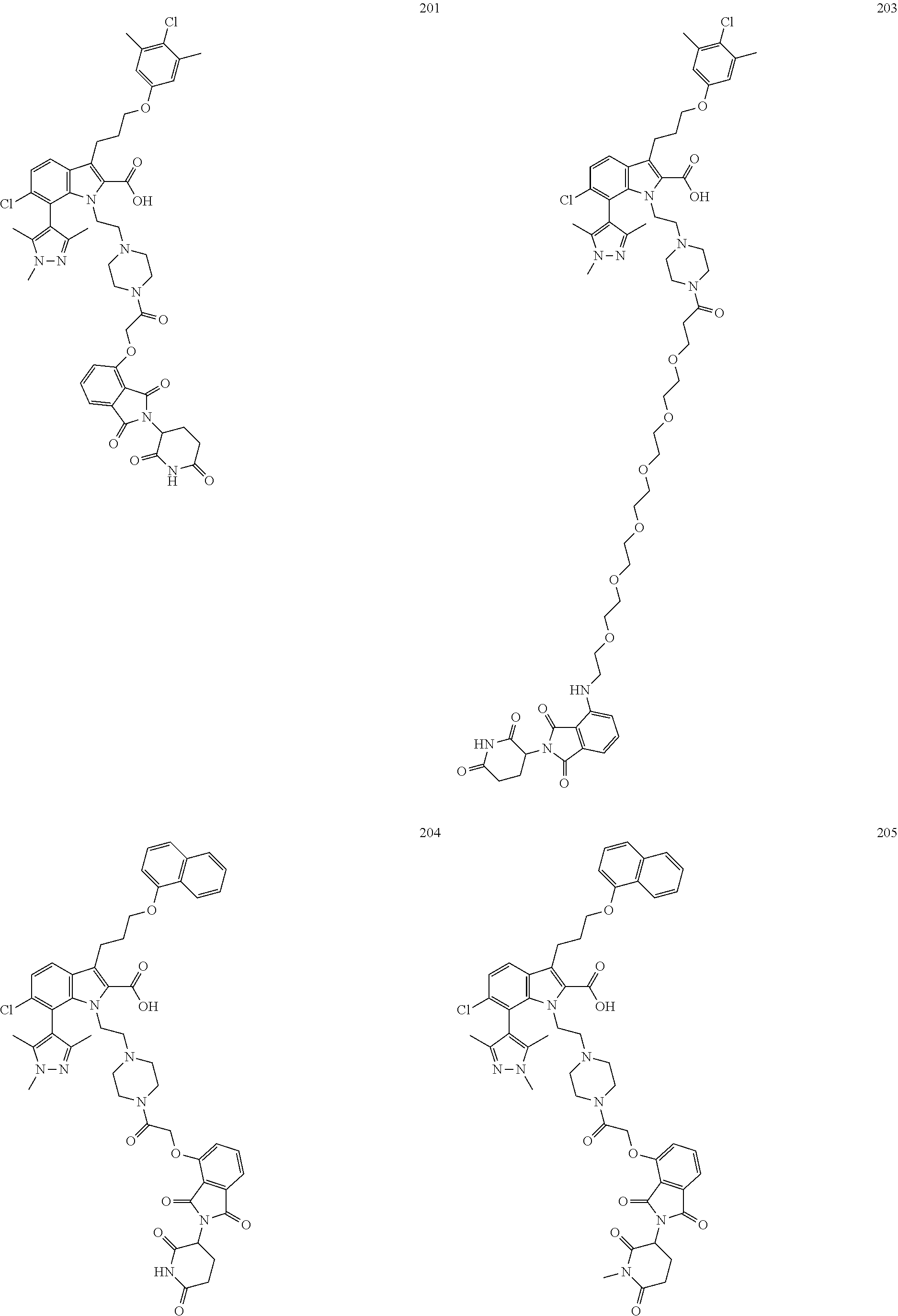 [SMILES: Cc1cc(OCCCc2c(C(=O)O)n(CCN3CCN(C(=O)COc4cccc5c4C(=O)N(C4CCC(=O)NC4=O)C5=O)CC3)c3c(-c4c(C)nn(C)c4C)c(Cl)ccc23)cc(C)c1Cl ; Cc1cc(OCCCc2c(C(=O)O)n(CCN3CCN(C(=O)CCOCCOCCOCCOCCOCCOCCNc4cccc5c4C(=O)N(C4CCC(=O)NC4=O)C5=O)CC3)c3c(-c4c(C)nn(C)c4C)c(Cl)ccc23)cc(C)c1Cl ; Cc1nn(C)c(C)c1-c1c(Cl)ccc2c(CCCOc3cccc4ccccc34)c(C(=O)O)n(CCN3CCN(C(=O)COc4cccc5c4C(=O)N(C4CCC(=O)NC4=O)C5=O)CC3)c12 ; Cc1nn(C)c(C)c1-c1c(Cl)ccc2c(CCCOc3cccc4ccccc34)c(C(=O)O)n(CCN3CCN(C(=O)COc4cccc5c4C(=O)N(C4CCC(=O)N(C)C4=O)C5=O)CC3)c12]

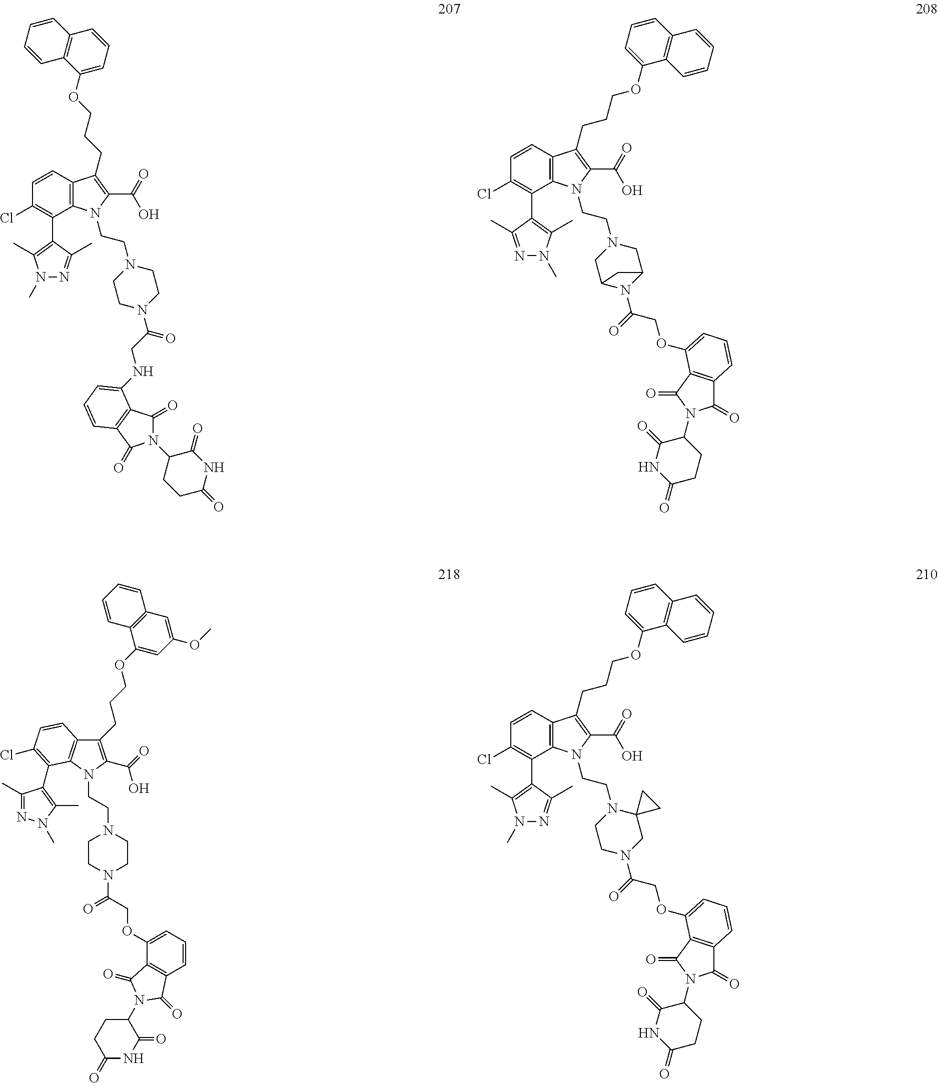

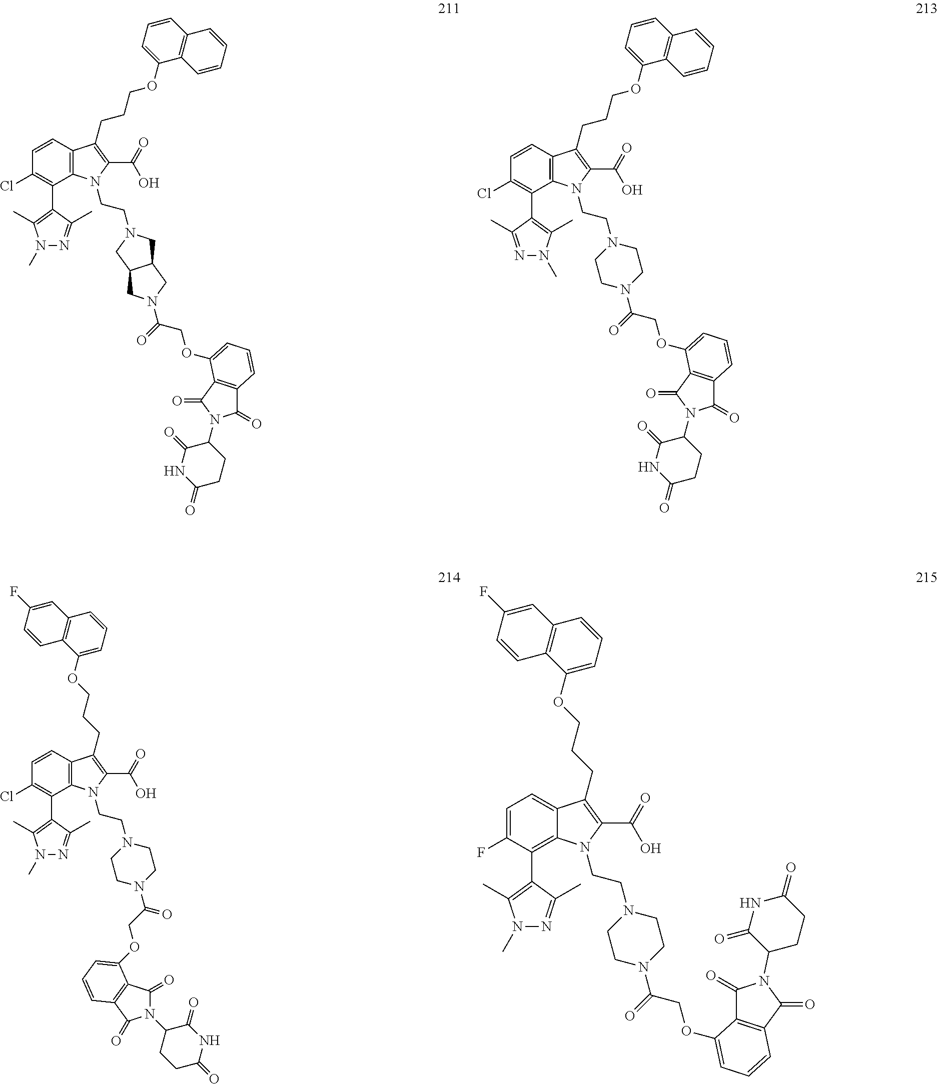 [SMILES: Cc1nn(C)c(C)c1-c1c(Cl)ccc2c(CCCOc3cccc4ccccc34)c(C(=O)O)n(CCN3C[C@@H]4CN(C(=O)COc5cccc6c5C(=O)N(C5CCC(=O)NC5=O)C6=O)C[C@@H]4C3)c12 ; Cc1nn(C)c(C)c1-c1c(Cl)ccc2c(CCCOc3cccc4ccccc34)c(C(=O)O)n(CCN3CCN(C(=O)COc4cccc5c4C(=O)N(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4cccc5c4C(=O)N(C4CCC(=O)NC4=O)C5=O)CC3)c12 ; Cc1nn(C)c(C)c1-c1c(F)ccc2c(CCCOc3cccc4cc(F)ccc34)c(C(=O)O)n(CCN3CCN(C(=O)COc4cccc5c4C(=O)N(C4CCC(=O)NC4=O)C5=O)CC3)c12]

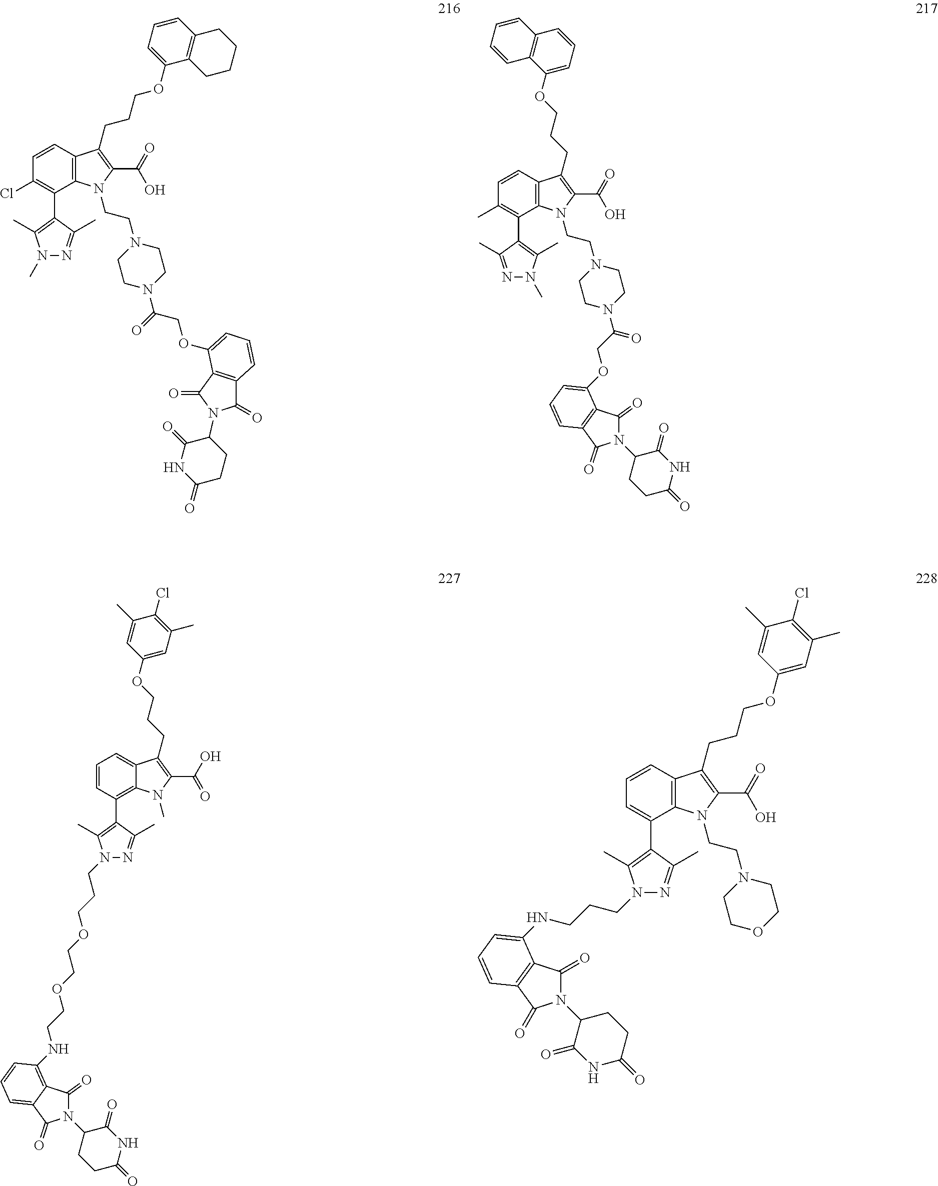 [SMILES: Cc1nn(C)c(C)c1-c1c(Cl)ccc2c(CCCOc3cccc4c3CCCC4)c(C(=O)O)n(CCN3CCN(C(=O)COc4cccc5c4C(=O)N(C4CCC(=O)NC4=O)C5=O)CC3)c12 ; Cc1ccc2c(CCCOc3cccc4ccccc34)c(C(=O)O)n(CCN3CCN(C(=O)COc4cccc5c4C(=O)N(C4CCC(=O)NC4=O)C5=O)CC3)c2c1-c1c(C)nn(C)c1C ; Cc1cc(OCCCc2c(C(=O)O)n(C)c3c(-c4c(C)nn(CCCOCCOCCNc5cccc6c5C(=O)N(C5CCC(=O)NC5=O)C6=O)c4C)cccc23)cc(C)c1Cl ; Cc1cc(OCCCc2c(C(=O)O)n(CCN3CCOCC3)c3c(-c4c(C)nn(CCCNc5cccc6c5C(=O)N(C5CCC(=O)NC5=O)C6=O)c4C)cccc23)cc(C)c1Cl]

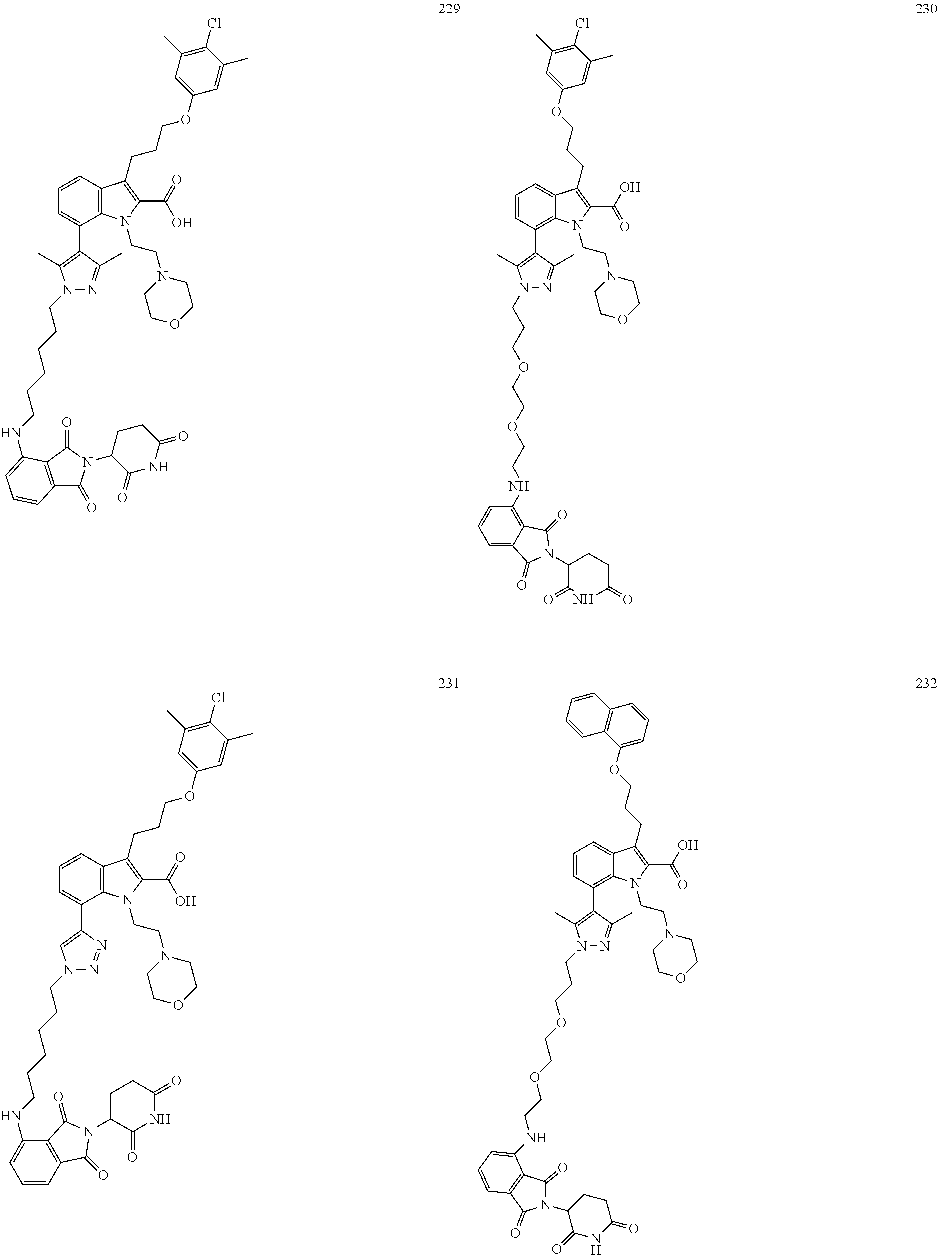 [SMILES: Cc1cc(OCCCc2c(C(=O)O)n(CCN3CCOCC3)c3c(-c4c(C)nn(CCCCCCNc5cccc6c5C(=O)N(C5CCC(=O)NC5=O)C6=O)c4C)cccc23)cc(C)c1Cl ; Cc1cc(OCCCc2c(C(=O)O)n(CCN3CCOCC3)c3c(-c4c(C)nn(CCCOCCOCCNc5cccc6c5C(=O)N(C5CCC(=O)NC5=O)C6=O)c4C)cccc23)cc(C)c1Cl ; Cc1cc(OCCCc2c(C(=O)O)n(CCN3CCOCC3)c3c(-c4cn(CCCCCCNc5cccc6c5C(=O)N(C5CCC(=O)NC5=O)C6=O)nn4)cccc23)cc(C)c1Cl ; Cc1nn(CCCOCCOCCNc2cccc3c2C(=O)N(C2CCC(=O)NC2=O)C3=O)c(C)c1-c1cccc2c(CCCOc3cccc4ccccc34)c(C(=O)O)n(CCN3CCOCC3)c12]

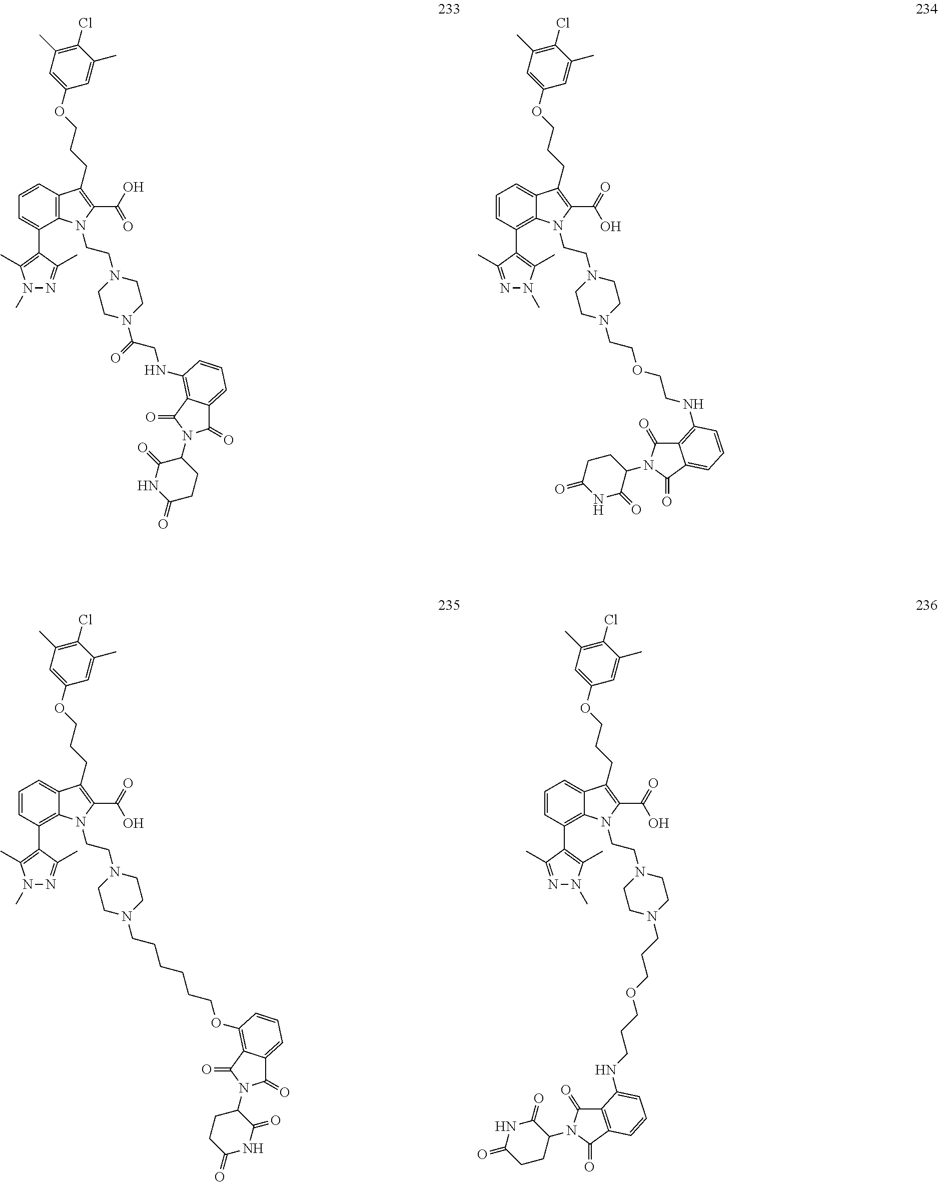 [SMILES: Cc1cc(OCCCc2c(C(=O)O)n(CCN3CCN(C(=O)CNc4cccc5c4C(=O)N(C4CCC(=O)NC4=O)C5=O)CC3)c3c(-c4c(C)nn(C)c4C)cccc23)cc(C)c1Cl ; Cc1cc(OCCCc2c(C(=O)O)n(CCN3CCN(CCOCCNc4cccc5c4C(=O)N(C4CCC(=O)NC4=O)C5=O)CC3)c3c(-c4c(C)nn(C)c4C)cccc23)cc(C)c1Cl ; Cc1cc(OCCCc2c(C(=O)O)n(CCN3CCN(CCCCCCOc4cccc5c4C(=O)N(C4CCC(=O)NC4=O)C5=O)CC3)c3c(-c4c(C)nn(C)c4C)cccc23)cc(C)c1Cl ; Cc1cc(OCCCc2c(C(=O)O)n(CCN3CCN(CCCOCCCNc4cccc5c4C(=O)N(C4CCC(=O)NC4=O)C5=O)CC3)c3c(-c4c(C)nn(C)c4C)cccc23)cc(C)c1Cl]

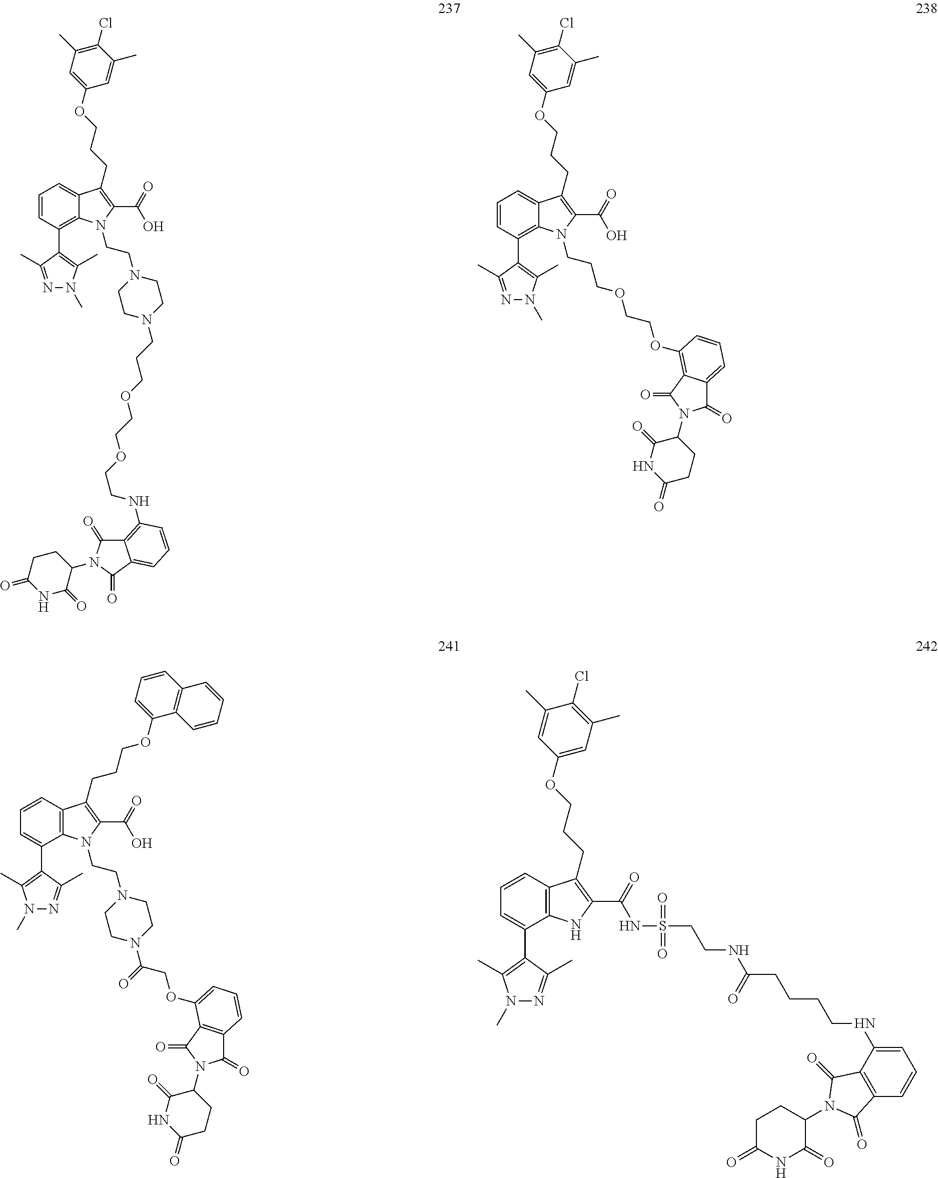 [SMILES: Cc1cc(OCCCc2c(C(=O)O)n(CCN3CCN(CCCOCCOCCNc4cccc5c4C(=O)N(C4CCC(=O)NC4=O)C5=O)CC3)c3c(-c4c(C)nn(C)c4C)cccc23)cc(C)c1Cl ; Cc1cc(OCCCc2c(C(=O)O)n(CCCOCCOc3cccc4c3C(=O)N(C3CCC(=O)NC3=O)C4=O)c3c(-c4c(C)nn(C)c4C)cccc23)cc(C)c1Cl ; Cc1nn(C)c(C)c1-c1cccc2c(CCCOc3cccc4ccccc34)c(C(=O)O)n(CCN3CCN(C(=O)COc4cccc5c4C(=O)N(C4CCC(=O)NC4=O)C5=O)CC3)c12 ; Cc1cc(OCCCc2c(C(=O)NS(=O)(=O)CCNC(=O)CCCCNc3cccc4c3C(=O)N(C3CCC(=O)NC3=O)C4=O)[nH]c3c(-c4c(C)nn(C)c4C)cccc23)cc(C)c1Cl]

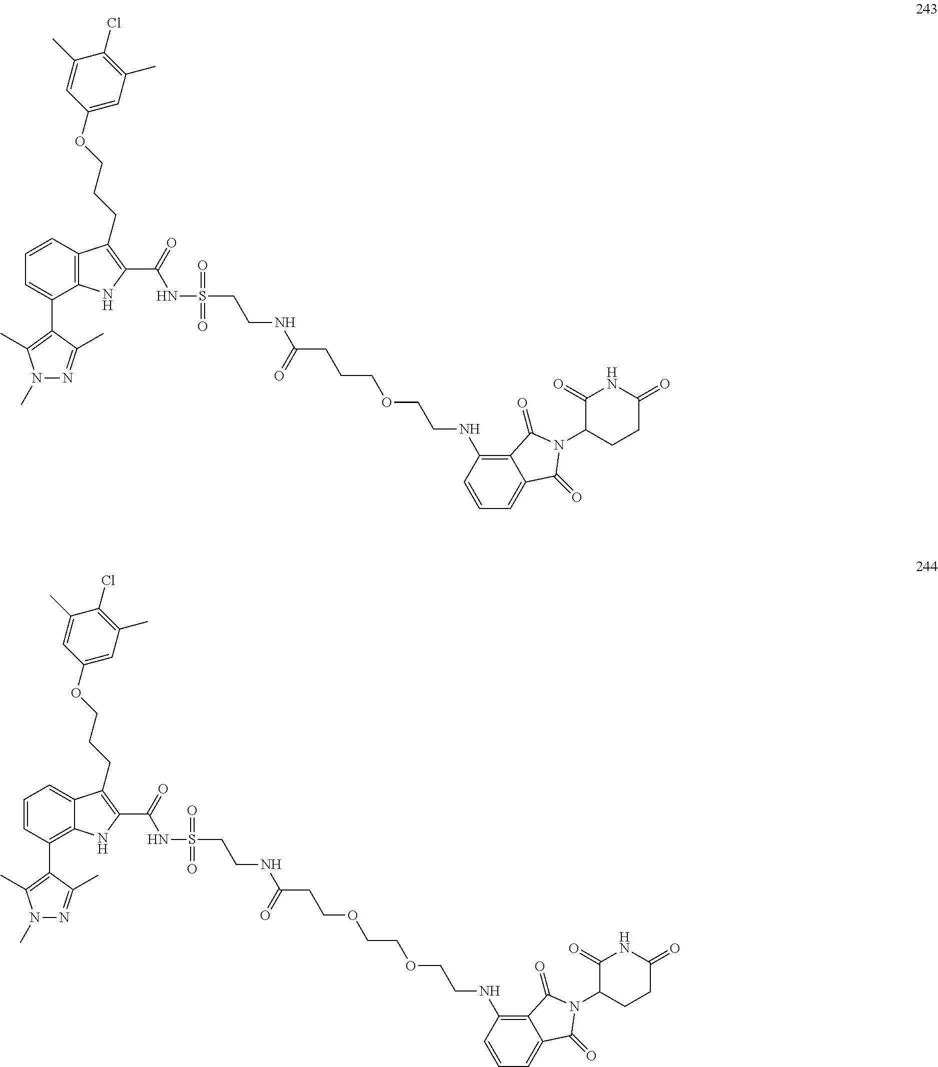 [SMILES: Cc1cc(OCCCc2c(C(=O)NS(=O)(=O)CCNC(=O)CCCOCCNc3cccc4c3C(=O)N(C3CCC(=O)NC3=O)C4=O)[nH]c3c(-c4c(C)nn(C)c4C)cccc23)cc(C)c1Cl ; Cc1cc(OCCCc2c(C(=O)NS(=O)(=O)CCNC(=O)CCOCCOCCNc3cccc4c3C(=O)N(C3CCC(=O)NC3=O)C4=O)[nH]c3c(-c4c(C)nn(C)c4C)cccc23)cc(C)c1Cl]

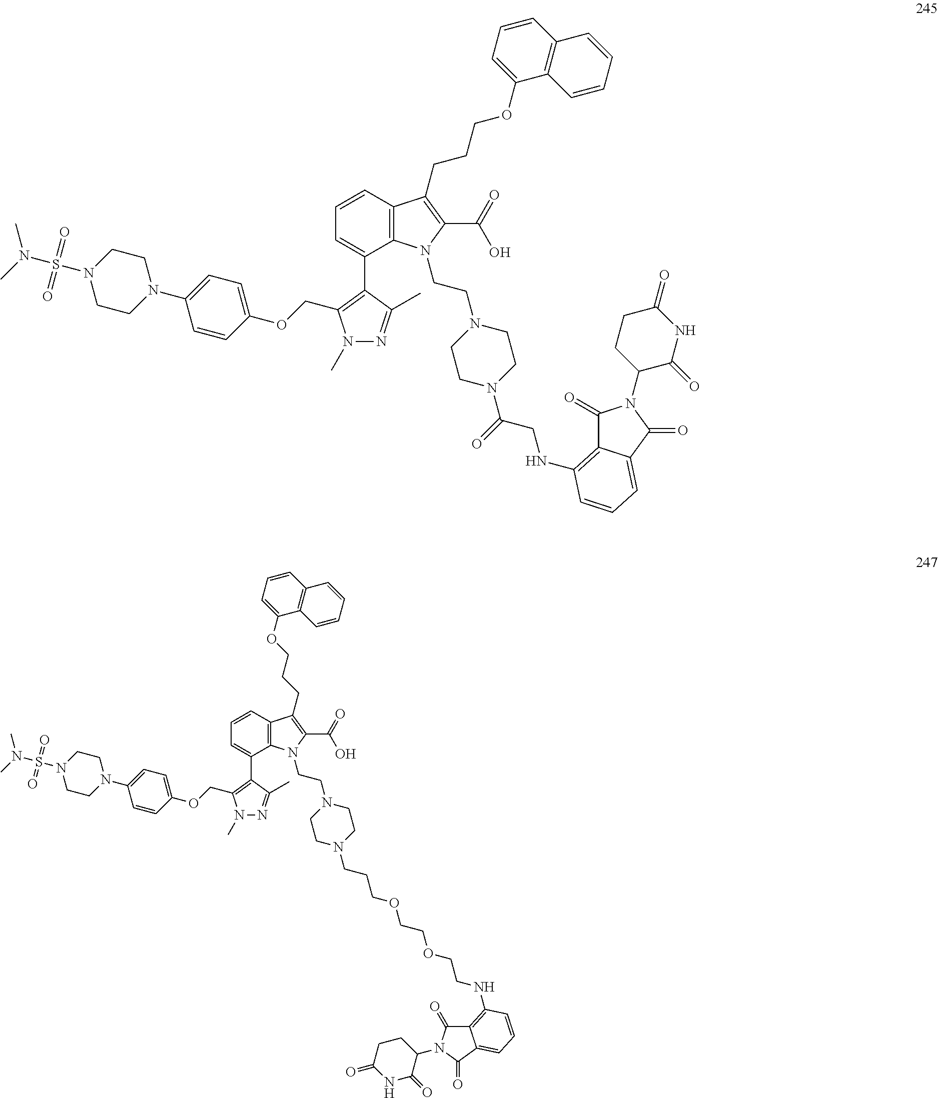 [SMILES: Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(C(=O)CNc4cccc5c4C(=O)N(C4CCC(=O)NC4=O)C5=O)CC3)c12 ; Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(CCCOCCOCCNc4cccc5c4C(=O)N(C4CCC(=O)NC4=O)C5=O)CC3)c12]

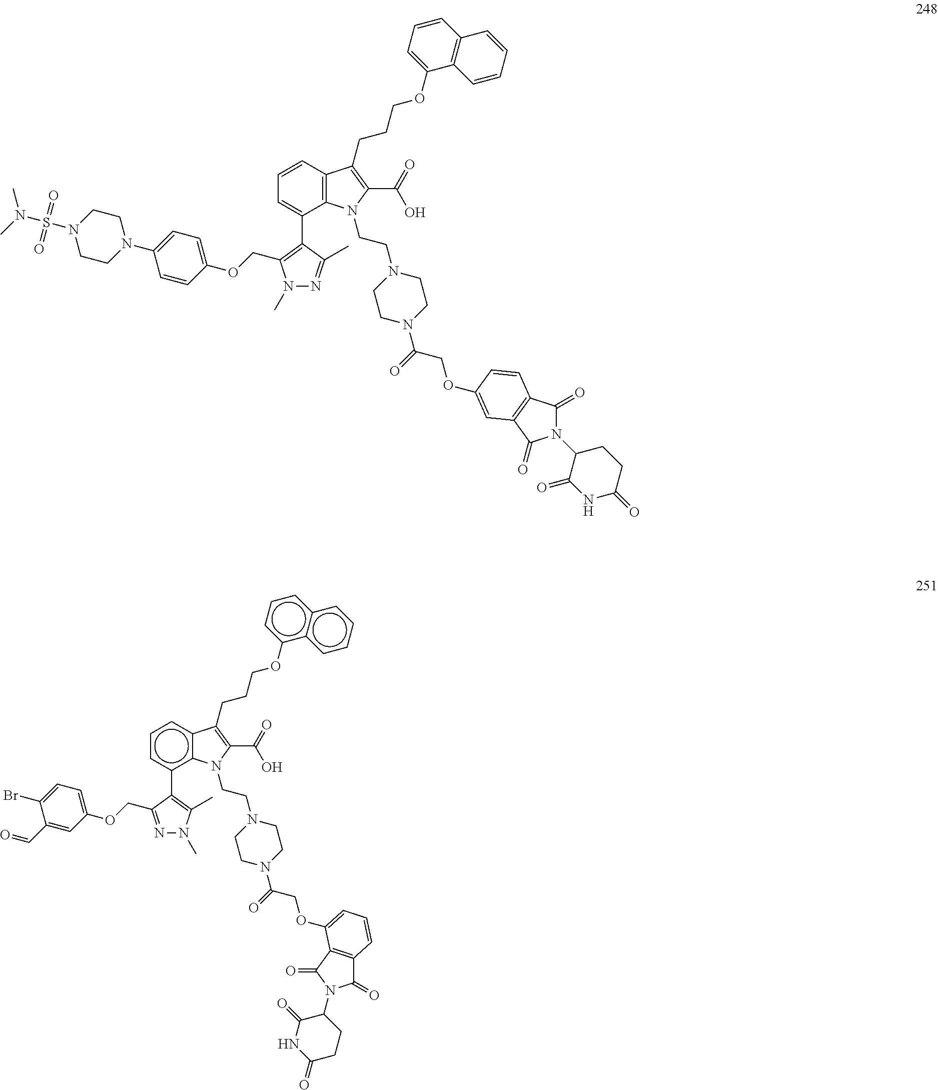

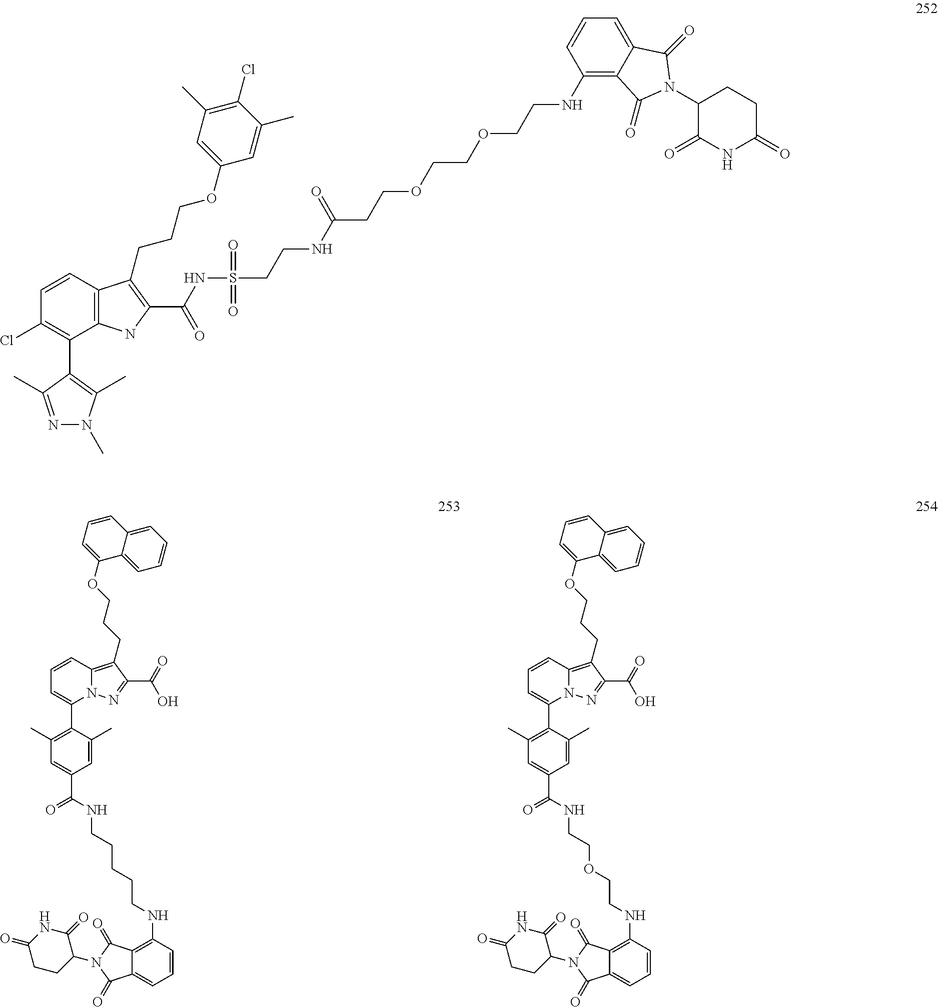 [SMILES: Cc1cc(OCCCc2c(C(=O)NS(=O)(=O)CCNC(=O)CCOCCOCCNc3cccc4c3C(=O)N(C3CCC(=O)NC3=O)C4=O)[nH]c3c(-c4c(C)nn(C)c4C)c(Cl)ccc23)cc(C)c1Cl ; Cc1cc(C(=O)NCCCCCNc2cccc3c2C(=O)N(C2CCC(=O)NC2=O)C3=O)cc(C)c1-c1cccc2c(CCCOc3cccc4ccccc34)c(C(=O)O)nn12 ; Cc1cc(C(=O)NCCOCCNc2cccc3c2C(=O)N(C2CCC(=O)NC2=O)C3=O)cc(C)c1-c1cccc2c(CCCOc3cccc4ccccc34)c(C(=O)O)nn12]

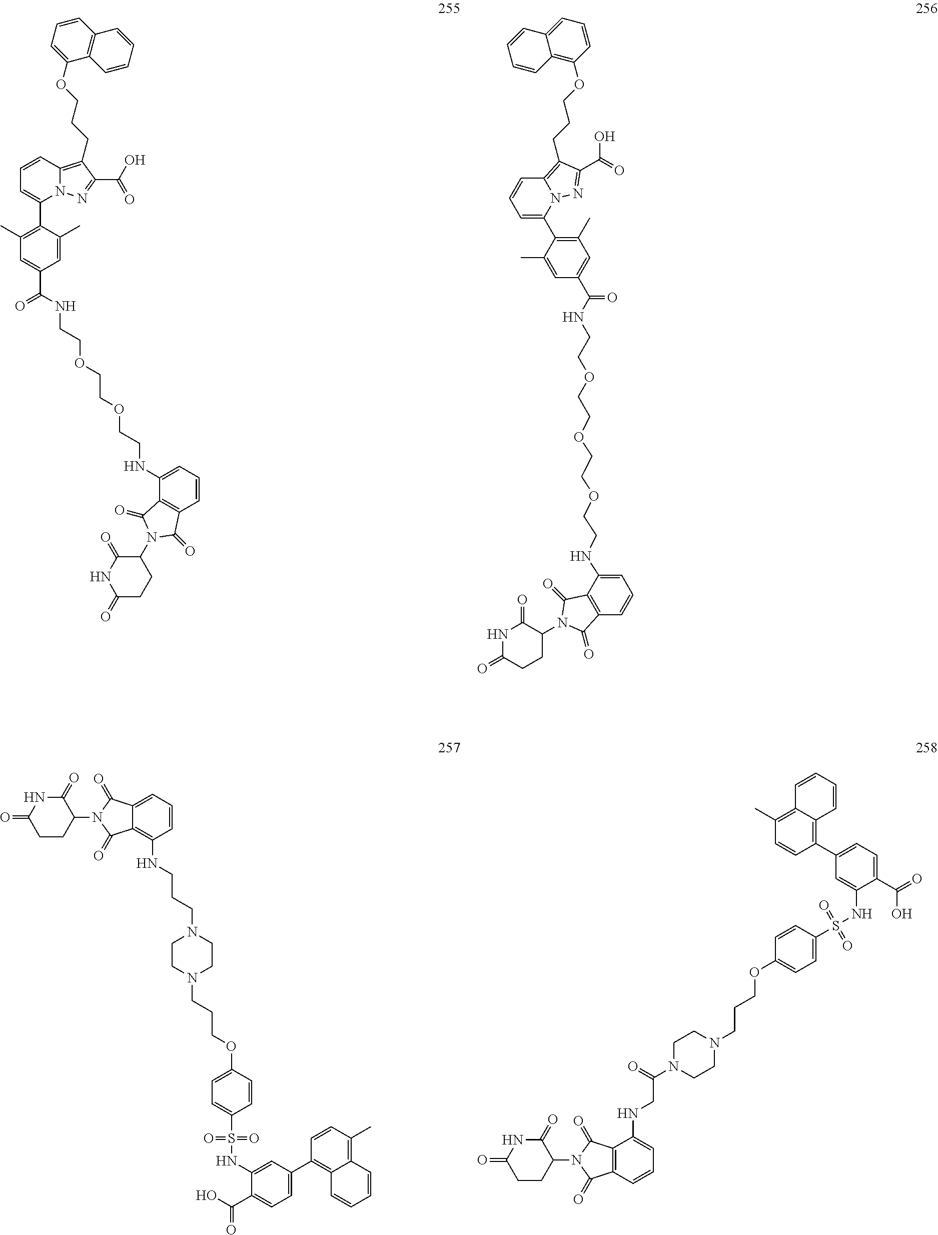 [SMILES: Cc1cc(C(=O)NCCOCCOCCNc2cccc3c2C(=O)N(C2CCC(=O)NC2=O)C3=O)cc(C)c1-c1cccc2c(CCCOc3cccc4ccccc34)c(C(=O)O)nn12 ; Cc1cc(C(=O)NCCOCCOCCOCCNc2cccc3c2C(=O)N(C2CCC(=O)NC2=O)C3=O)cc(C)c1-c1cccc2c(CCCOc3cccc4ccccc34)c(C(=O)O)nn12 ; Cc1ccc(-c2ccc(C(=O)O)c(NS(=O)(=O)c3ccc(OCCCN4CCN(CCCNc5cccc6c5C(=O)N(C5CCC(=O)NC5=O)C6=O)CC4)cc3)c2)c2ccccc12 ; Cc1ccc(-c2ccc(C(=O)O)c(NS(=O)(=O)c3ccc(OCCCN4CCN(C(=O)CNc5cccc6c5C(=O)N(C5CCC(=O)NC5=O)C6=O)CC4)cc3)c2)c2ccccc12]

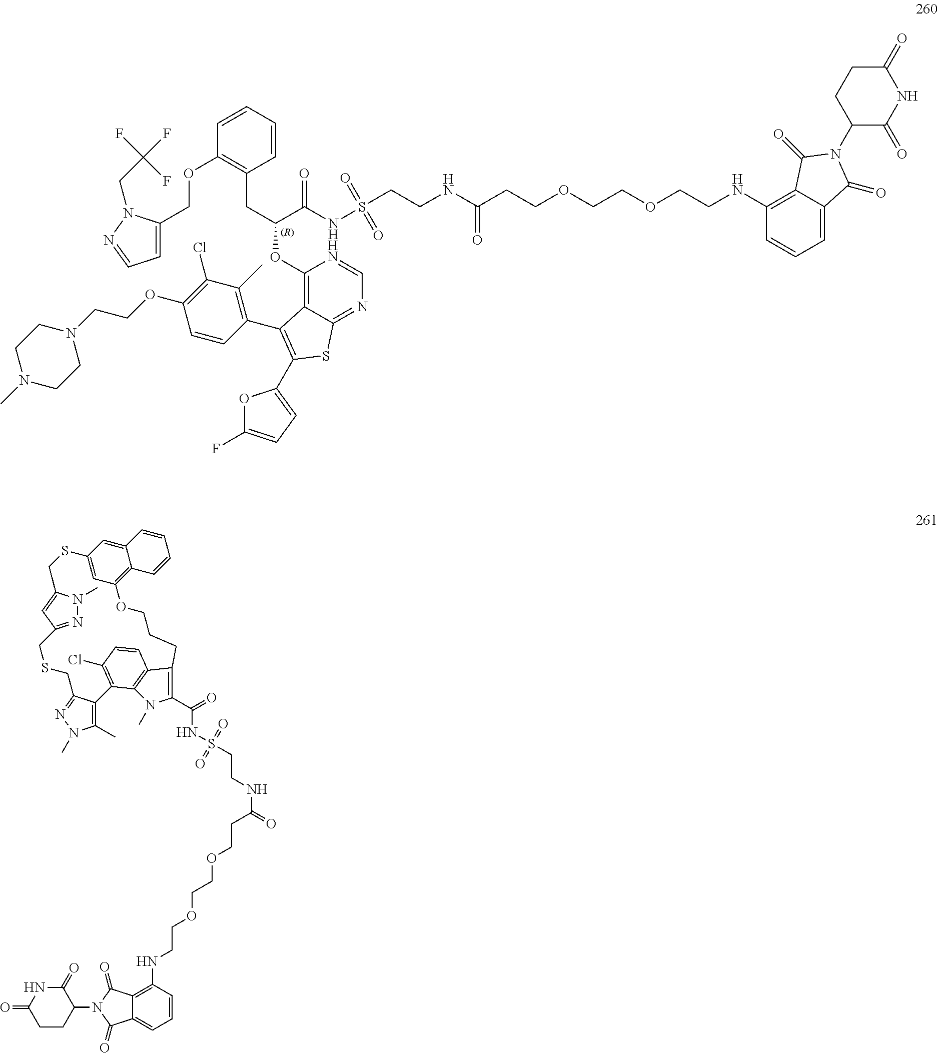

In some embodiments, each alkyl, alkenyl, alkynyl, aryl, heteroaryl and benzyl is unsubstituted.

In accordance with a second aspect of the invention, there is provided a compound of formula (I)

[MCL-1 ligand moiety]-[linker]-[ligase ligand moiety] (I)

or a salt, solvate, hydrate, isomer or prodrug thereof,
wherein [ligase ligand moiety] is:
(a) Formula (IV)

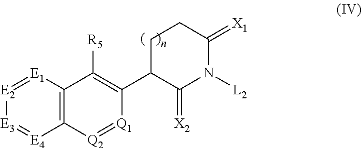

wherein:
each of X₁ and X₂ is independently O or S;
each of Q₁ and Q₂ is independently N or CR⁵, wherein at least one of Q₁ and Q₂ is N;
each of E₁, E₂, E₃ and E₄ is independently N or CR′;
n is 0, 1 or 2;
L₂ is hydrogen, alkyl, alkenyl, aryl, heteroaryl, benzyl, haloalkyl, haloalkenyl, —C(O)R′″, —C(O)OR′″, —C(O)NH₂, —C(O)NHR′″, —C(O)NR′″₂, —OR′″, —NR′″₂, or —S(O)₂R′;
each R⁵ is independently hydrogen, halogen, alkyl, alkenyl, alkynyl, aryl, heteroaryl, benzyl, haloalkyl, haloalkenyl, —NH₂, —NHR′″, —NR″₂, —NR′″C(O)R′″, —NR′″C(O)OR′″, —NO₂, —CN, —C(O)R′″, —C(O)OR′″, —C(O)NH₂, —C(O)NHR′″, —C(O)NR′″₂, —OR′″, —OC(O)R′″, —OC(O)OR′″, —OC(O)NH₂, —OC(O)NHR′″, —OC(O)NR′″₂, —SR′″, —S(O)₂R′″, —S(O)₂OR′″, —S(O)₂NH₂, —S(O)₂NHR′″, —S(O)₂NR′″₂; —O—R²¹, —NH—R²¹, —C(O)—NH—R²¹, or —CH₂—NH—C(O)—R²¹;
each R′ is independently hydrogen, halogen, alkyl, alkenyl, alkynyl, aryl, heteroaryl, benzyl, haloalkyl, haloalkenyl, —NH₂, —NHR′″, —NR′″₂, —NR′″C(O)R′″, —NR′″C(O)OR′″, —NO₂, —CN, —C(O)R′″, —C(O)OR′″, —C(O)NH₂, —C(O)NHR′″, —C(O)NR′″₂, —OR′″, —OC(O)R′″, —OC(O)OR′″, —OC(O)NH₂, —OC(O)NHR′″, —OC(O)NR′″₂, —SR′″, —S(O)₂R′″, —S(O)₂OR′″, S(O)₂NH₂, —S(O)₂NHR′″, —S(O)₂NR′″₂, —R²¹, —O—R²¹, —NH—R²¹, —C(O)—R²¹, —C(O)—NH—R²¹, or —CH₂—NH—C(O)—R²¹;
and
each R′″ is independently hydrogen, alkyl, alkenyl, aryl, heteroaryl, or benzyl;
   wherein R²¹ is a bond connected to R¹⁸ of the linker, and wherein Formula (IV) contains a single R²¹; or
(b) Formula (Va) or (Vb):

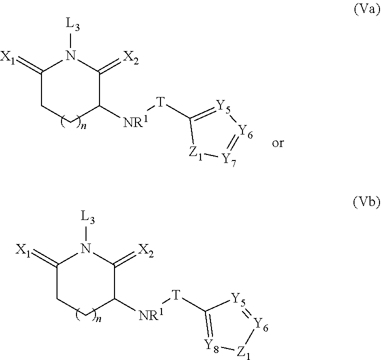

or a pharmaceutically acceptable salt or tautomer thereof,
wherein
each of X₁ and X₂ is independently O or S;
Z₁ is O, S or NR⁶;
T is is C═O or SO₂;
R′ is hydrogen, alkyl, cycloalkyl, alkenyl, cycloalkenyl, aryl, heteroaryl, or benzyl;
each of Y₅, Y₆, Y₇, and Y₈ is independently N or CR⁷,
wherein at least one of Y₅, Y₆ and Y₇ in Formula (Va) is CR⁷, and at least one of Y₅, Y₅ and Y₈ in Formula (Vb) is CR⁷;
n is 0, 1 or 2;
L₃ is hydrogen, alkyl, alkenyl, aryl, heteroaryl, benzyl, haloalkyl, haloalkenyl, —C(O)R″″, —CH₂C(O)OR″″, —C(O)OR″″, —C(O)NH₂, —C(O)NHR″″, —C(O)NR″″₂, —OR″″, —NR″″₂, or —S(O)₂R″″;
each R⁷ is independently hydrogen, halogen, alkyl, cycloalkyl, heterocycloalkyl, alkenyl, cycloalkenyl, alkynyl, aryl, heteroaryl, benzyl, haloalkyl, haloalkenyl, —NH₂, —NHR″″, —NR″″₂, —CH₂NR″″₂, —NR″″C(O)R″″, —NR″″C(O)CH₂NR″″₂, —NR″″C(O)CH₂-heterocycloalkyl, —NR″″C(O)CH(OH)R″″, —CH₂NR″″C(O)OR″″, —NR″″C(O)OR″″, —NR″″SO₂R″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —NHC(S)NHR″″, SR″″, or —S(O)₂R″″, —S(O)₂OR″″, —S(O)₂NH₂, —S(O)₂NHR″″, —S(O)₂NR″″₂, —O—R²¹, —NH—R²¹, —C(O)—NH—R²¹, or —CH₂—NH—C(O)—R²¹;
each R″″ is independently hydrogen, alkyl, cycloalkyl, alkenyl, cycloalkenyl, aryl, heteroaryl, or benzyl;
R⁶ is hydrogen, alkyl, cycloalkyl, alkenyl, cycloalkenyl, alkynyl, aryl, heteroaryl, benzyl, haloalkyl, haloalkenyl, —NH₂, —NHR″″, —NR″″₂, —NR″″C(O)R″″, —N[C(O)R″″]₂, —NR″″C(O)OR″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —SR″″, or —S(O)₂R″″, —S(O)₂OR″″, —S(O)₂NH₂, —S(O)₂NHR″″, —S(O)₂NR″″₂, —R²¹, —C(O)—NH—R²¹, or —CH₂—NH—C(O)—R²¹;
wherein R²¹ is a bond connected to R¹⁸ of the linker, and wherein formula (Va) and formula (Vb) each contain a single R²¹;
wherein when Z₁ is O, then Y₆ is CR⁷ and
   wherein when the compound is of Formula (Va), then
(i) when each of Y₅, Y₆ and Y₇ is CR⁷, then at least one of R⁷ is not H;
(ii) when Z₁ is NR⁶, then Y₆ and Y₇ are CR⁷;
(iii) when Z₁ is S, then Y₅ is not C—OMe and Y₆ is not C—OMe;
(iv) when Z₁ is S and Y₅ is C—NHCOMe, then Y₇ is not C—CH₂NR″″C(O)OR″″;
(v) when Z₁ is S and Y₅ is N, then Y₆ is not C—H, C-aryl or C—C(O)OR″″; and
(vi) when Z₁ is S and Y₆ is N, then Y₇ is C—NH₂, C—NHR″″, C—NR″″₂, C—NR″″C(O)OR″″, C—CH₂NR″″C(O)OR″″, C-haloalkyl, C-^{t}Butyl, C—OR″″, C—COOR″″ or C—SR″″; wherein when Y₇ is C—NH₂, C—NHR″″ or C—NR″″₂, then Y₅ is C—H;
   and when the compound is of Formula (Vb), then:
(vii) when each of Y₅, Y₆ and Y₈ is CR⁷, then at least one of R⁷ is not H;
(viii) when Z₁ is S, then Y₅ is not C—COOH or C—NHC(O)Me, and Y₈ is not C—Br;
(ix) when Z₁ is S and Y₆ is C—Br, then Y₈ is C—OR″″
(x) when Z₁ is S, Y₅ is N and Y₆ is C—H or C—NH₂, then Y₈ is not C—H
(xi) when Z₁ is S and Y₅ is N, then Y₆ is not C— halogen, C-alkyl, C-cycloalkyl, C-aryl, C-heteroaryl, C—CH₂NH₂, C—COOalkyl, or C—NHC(O)alkyl; (xii) when Z₁ is NR⁶, then Y₅, Y₆ and Y₈ are CR⁷.
   or
(c) Formula (IIa) or (IIb):

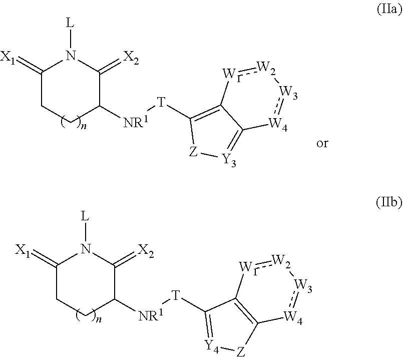

wherein
each of X₁ and X₂ is independently O or S;
Z is O, S or NR²;
T is C═O or SO₂;
Y₃ is N or CR;
Y₄ is N or CR;
[symbol] indicates a single or double bond, wherein
   when each [symbol] is a double bond, each of W₁, W₂, W₃ and W₄ is independently N or CR^{a}, wherein at least one of W₁, W₂, W₃ and W₄ is N, and
   when each [symbol] is a single bond, W₁, W₂, W₃ and W₄ are each CR^{a}₂ and Y₄ is CR; n is 0, 1 or 2;
L is hydrogen, alkyl, alkenyl, aryl, heteroaryl, benzyl, haloalkyl, haloalkenyl, —C(O)R^{h}, —C(O)OR^{h}, —C(O)NH₂, —C(O)NHR^{h}, —C(O)NR^{h}₂, —OR^{h}, —NR^{h}₂, or —S(O)₂R^{h};
each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH₂R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, or —S(O)₂R^{h}, —S(O)₂OR^{h}, —S(O)₂NH₂, —S(O)₂NHR^{h}, or —S(O)₂NR^{h}₂;
each R^{a} is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, —S(O)₂NR^{h}₂, —O—R²¹, —NH—R²¹, —C(O)—NH—R²¹, or —CH₂—NH—C(O)—R²¹;
each R^{h} is independently hydrogen, alkyl, cycloalkyl, heterocycloalkyl, alkenyl, cycloalkenyl, aryl, heteroaryl, or benzyl;
R² is hydrogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —N[C(O)R^{h}]₂, —NR^{h}C(O)OR^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, or —S(O)₂NR^{h}₂; and
R¹ is hydrogen, alkyl, cycloalkyl, alkenyl, cycloalkenyl, aryl, heteroaryl, or benzyl;
R²¹ is a bond connected to R¹⁸ of the linker, and wherein formula (IIa) and formula (IIb) each contain a single R²¹;
wherein when each [symbol] is a double bond, Z is NR², R² is hydrogen, and each R^{a} is hydrogen, then W₄ is CR^{a};
   wherein
   [MCL-1 ligand moiety] is a compound of Formula (A), Formula (B) or Formula (C)

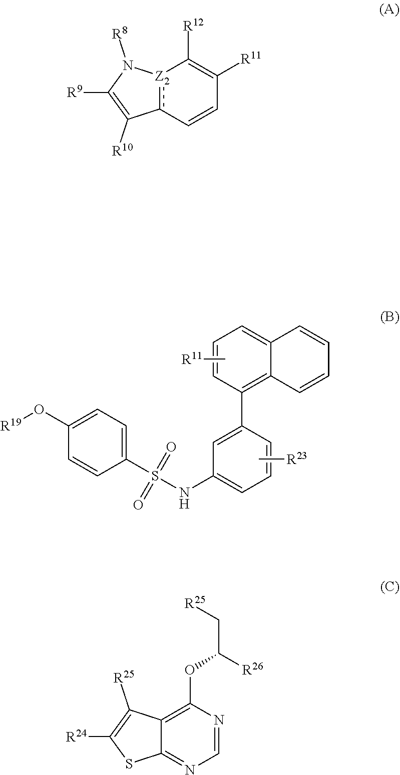

wherein
[symbol] is a single bond or a double bond;
R⁸ is H, R¹⁹, or C₁-C₆ alkyl optionally substituted with morpholine;
R⁹ is —C(O)OH, —C(O)OC₁-C₆alkyl, —C(O)NH₂, —C(O)OR¹⁹ or —C(O)NHR¹⁹,
R¹⁰ is —C₂₋₅alkyl-O—R¹³ or —C₂₋₅alkyl-NMe-R¹³ wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹¹ is H, halogen or C₁-C₆ alkyl,
R¹² is H,

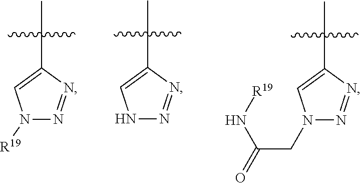

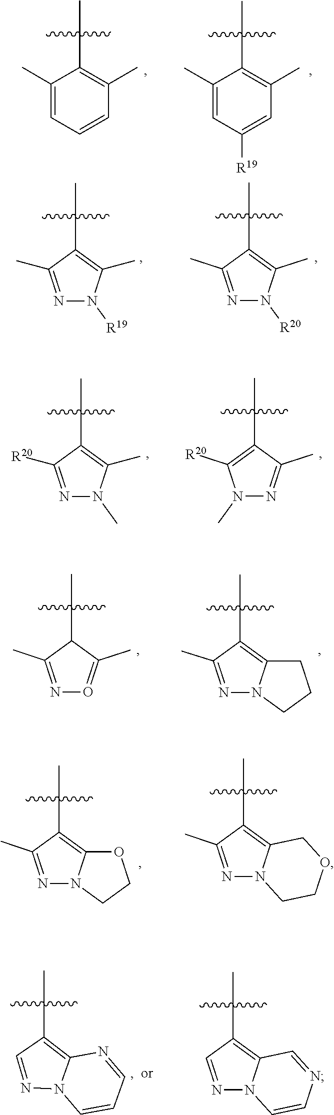

wherein R²⁰ is Me, —CH₂—OMe, —CH₂—O-bromobenzaldehyde, or

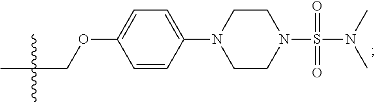 [SMILES: *COc1ccc(N2CCN(S(=O)(=O)N(C)C)CC2)cc1]

or when R¹² is

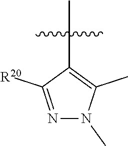

and R¹⁰ is —O-naphthyl substituted with —O— or —S—, then R²⁰ is

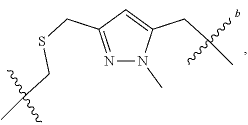 [SMILES: *CSCc1cc(C*)n(C)n1]

wherein [symbol] indicates attachment to —O— or —S— of R¹⁰;
and wherein
R¹⁹ is a bond connected to R¹⁴ of the linker;
R²³ is —C(O)OH or —C(O)OC₁-C₆alkyl;
Z₂ is N or C, wherein when Z₂ is N, then [symbol] is a single bond; and when Z₂ is C, then [symbol] is a double bond,
R²⁴ is furan optionally substituted with at least one halogen,
each R²⁵ is independently phenyl substituted with —OR²⁸ and optionally further substituted with at least one substituent selected from halogen and C₁-C₆ alkyl;
R²⁶ is —C(O)OR¹⁹ or —C(O)NHR¹⁹; and
each R²⁸ is independently —C₁₋₃alkyl-(N-alkyl piperazine) or —C₁₋₃alkyl-(N-haloalkylpyrazole)
and wherein each of Formula (A), Formula (B) and Formula (C) contains a single R¹⁹;
   and wherein [linker] has the following formula

R¹⁴—R¹⁵—R¹⁶—R¹⁷—R¹⁸

wherein
R¹⁴ is —C₁₋₆alkyl, —C₂₋₆alkenyl, —C₂₋₆alkynyl, C₁₋₆ alkyl-N(C₁₋₆ alkyl)-, —C(O)—, —SO₂— or is absent
R¹⁵ is cycloalkyl, heterocycloalkyl, aryl, heteroaryl, —C₁₋₆ alkyl-NH—, C₁₋₆ alkyl-N(C₁₋₆ alkyl)-, -cycloalkyl-NH—, -heterocycloalkyl-NH—, or is absent
R¹⁶ is —C₁₋₆alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, (C₂H₄—O)ₓ, (C₃H₆—O)ₓ, or is absent
x is 1-10
y is 2-10
R¹⁸ is —C₁₋₆ alkyl, heterocycloalkyl, or is absent
   wherein at least one of R¹⁴-R¹⁸ is present.

In some embodiments, each alkyl, alkenyl, alkynyl, aryl, heteroaryl and benzyl group is unsubstituted.

In some embodiments, each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR″″, —NR″″₂, —NR″″C(O)R″″, —NR″″C(O)CH(OH)R″″, —NR″″C(O)OR″″, —NR″″SO₂R″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —SR″″, or —S(O)₂R″″, —S(O)₂OR″″, —S(O)₂NH₂, —S(O)₂NHR″″, or —S(O)₂NR″″₂, —O—R²¹, —NH—R²¹, —C(O)—NH—R²¹, or —CH₂—NH—C(O)—R²¹.

In some embodiments, each R′ is independently hydrogen, halogen, alkyl, alkenyl, alkynyl, aryl, heteroaryl, benzyl, haloalkyl, haloalkenyl, —NH₂, —NHR′″, —NR′″₂, —NR′″C(O)R′″, —NR′″C(O)OR′″, —NO₂, —CN, —C(O)R′″, —C(O)OR′″, —C(O)NH₂, —C(O)NHR′″, —C(O)NR′″₂, —OR′″, —OC(O)R′″, —OC(O)OR′″, —OC(O)NH₂, —OC(O)NHR′″, —OC(O)NR′″₂, —SR′″, —S(O)₂R′″, —S(O)₂OR′″, S(O)₂NH₂, —S(O)₂NHR′″, —S(O)₂NR′″₂, —O—R²¹, —NH—R¹, —C(O)—NH—R¹, or —CH₂—NH—C(O)—R¹.

In some embodiments, R¹ is hydrogen.

In some embodiments, R⁶ is hydrogen.

In some embodiments, when Z₁ is S in Formula (Vb), then Y₅ is not C—NHC(O)R″″ or —C(O)OR″″.

In some embodiments, Z₁ is NR⁶.

In some embodiments, [ligase ligand moiety] is of Formula (Va) and Y₅, Y₆ and Y₇ are each CR⁷. In some such embodiments, Y₅ is —C—NHC(O)R″″, Y₆ is CH, and Y₇ is CH or CCl. In some such embodiments, L₃ is hydrogen; Z₁ is S; R¹ is hydrogen; T is C═O; and Y₇ is CH.

In some embodiments, the compound is of Formula (Vb) and Y₅, Y₆ and Y₅ are each CR⁷. In some such embodiments: L₃ is hydrogen; Z₁ is S; R¹ is H; T is C═O; Y₅ is CH, C—OR″″, CCl, C—CN, or C—NHC(O)R″″; Y₆ is CH, CCl, C-alkyl, C-cycloalkyl, or C-haloalkyl; and Y₅ is CH, C—OR″″, C—NHC(O)R″″, C—NHC(O)OR″″, C—NHR″″, C—NH₂, or C—NHSO₂R″″; wherein, when Y₅ is CCl, then Y₆ is CH, C-alkyl, C-cycloalkyl, or C-haloalkyl. In some such embodiments, each R″″ is independently alkyl, cycloalkyl, aryl or benzyl. In some embodiments, Y₅ is CH; Y₆ is CH or CCl; and Y₅ is C—OR″″ or C—NH₂. In some such embodiments, Y₅ is C—OMe or C—NH₂.

In some embodiments, Z is NR². In other embodiments, Z is S.

In some embodiments, each [symbol] is a double bond.

In some embodiments, L is hydrogen.

In some embodiments, one of W₁, W₂, W₃ and W₄ is N, and the remaining three of W₁, W₂, W₃ and W₄ are each CR^{a}. In some such embodiments, W₄ is CR^{a}. In other embodiments, two of W₁, W₂, W₃ and W₄ is N, and the remaining two of W₁, W₂, W₃ and W₄ are each CR^{a}. In other embodiments, one of W₁, W₂, W₃ and W₄ is CR^{a}, and the remaining three of W₁, W₂, W₃ and W₄ are each N.

In some embodiments, each R is independently hydrogen, halogen or —NR^{h}C(O)R^{h}.

In some embodiments, [ligase ligand moiety] is

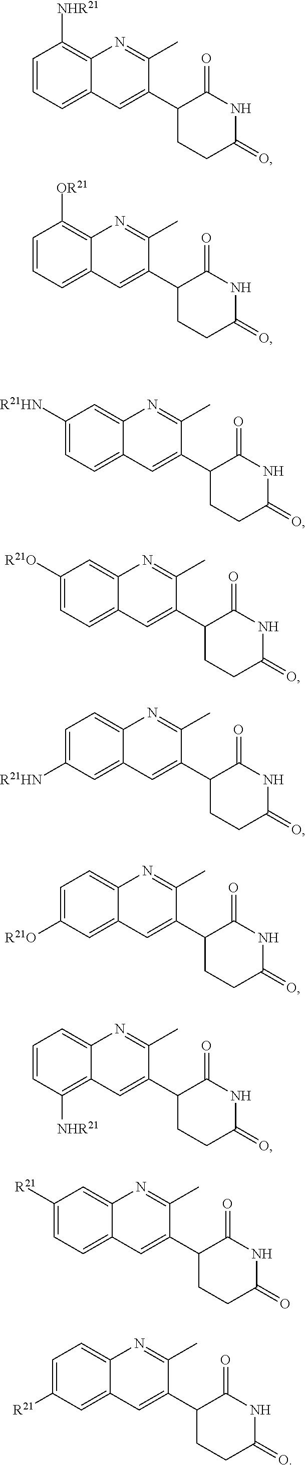

In some embodiments, [ligase ligand moiety] is:

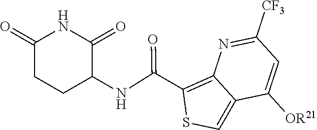

In some embodiments, E₁, E₂, E₃ and E₄ are each CR′.

In some embodiments, one of E₁, E₂, E₃ and E₄ is N and the remaining three of E₁, E₂, E₃ and E₄ are each CR′.

In some embodiments, Q₁ is CR⁵. In other embodiments, Q₂ is CR⁵

In some embodiments, R¹⁴ is —C₁₋₆alkyl, —C₂₋₆alkenyl, —C₂₋₆alkynyl, —C(O)—, —SO₂— or is absent.

In some embodiments, R¹⁵ is cycloalkyl, heterocycloalkyl, aryl, heteroaryl, —C₁₋₆ alkyl-NH—, -cycloalkyl-NH— or is absent.

In some embodiments,
R¹⁴ is —C₁₋₆ alkyl, —SO₂— or is absent
R¹⁵ is piperazine, bridged piperazine, piperazine N-oxide, piperazine cation, —C₁₋₆ alkyl-NH—,

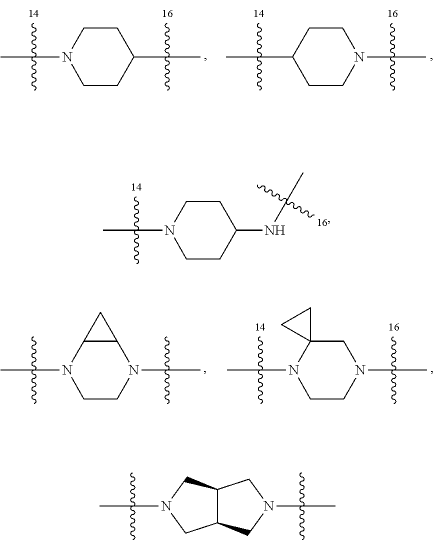 [SMILES: *C1CCN(*)CC1 ; *C1CCN(*)CC1 ; *NC1CCN(*)CC1 ; *N1CCN(*)C2CC21 ; *N1CCN(*)C2(CC2)C1 ; *N1C[C@H]2CN(*)C[C@H]2C1]

or is absent, wherein [symbol] indicates attachment to R¹⁴ and [symbol] indicates attachment to R¹⁶,
R¹⁶ is —C₁₋₆ alkyl, —C(O)—, —C(O)—NH—, —CH₂—C(O)—NH— or is absent
R¹⁷ is —CH₂(C₂H₄—O)_{y}, (C₂H₄—O)ₓ, (C₃H₆—O)ₓ, or is absent
x is 1-6
y is 2-6
R¹⁸ is —C₁₋₆ alkyl, piperazine, or is absent
   wherein at least one of R¹⁴-R¹⁸ is present.

In some embodiments, R¹⁸ is —C₁₋₆ alkyl or is absent.

In some embodiments, when R¹⁵ is piperazine, bridged piperazine, piperazine N-oxide, piperazine cation, —C₁₋₆ alkyl-NH—,

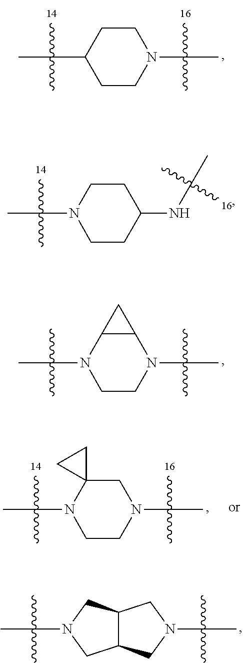 [SMILES: *C1CCN(*)CC1 ; *NC1CCN(*)CC1 ; *N1CCN(*)C2CC21 ; *N1CCN(*)C2(CC2)C1 ; *N1C[C@H]2CN(*)C[C@H]2C1]

then R¹⁴ is —C₁₋₆ alkyl.

In some embodiments,
R¹⁴ is —C₁₋₆ alkyl,
R¹⁵ is piperazine, bridged piperazine, piperazine N-oxide,

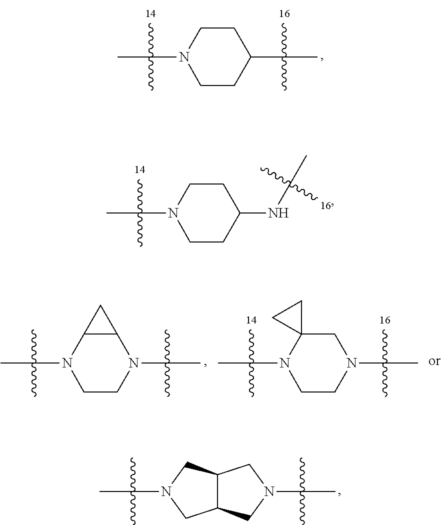 [SMILES: *C1CCN(*)CC1 ; *NC1CCN(*)CC1 ; *N1CCN(*)C2CC21 ; *N1CCN(*)C2(CC2)C1 ; *N1C[C@H]2CN(*)C[C@H]2C1]

R¹⁶ is —C(O)—, —CH₂—C(O)—NH—, or is absent
R¹⁷ is —CH₂(C₂H₄—O)_{y}, (C₂H₄—O)ₓ, (C₃H₆—O)ₓ, or is absent
R¹⁸ is —C₁₋₆ alkyl,
   wherein when R¹⁶ and R¹⁷ are absent, R¹⁸ is —C₃₋₆ alkyl. In some such embodiments, R¹⁴ is —C₂ alkyl; x is 1, 2 or 6; and y is 2. In some such embodiments, R¹⁵ is piperazine, R¹⁶ is —C(O)—, and R¹⁷ is absent. In some such embodiments, R¹⁴ is —C₂ alkyl, and R¹⁸ is —C₁₋₂ alkyl.

In some embodiments, when R¹⁴ is —SO₂—, at least two of R¹⁵-R¹⁸ are present, and at least one of R¹⁵—R¹⁸ is not C₁₋₆ alkyl.

In some embodiments, R¹⁴ is —SO₂—; R¹⁵ is —C₁₋₆ alkyl-NH—; R¹⁶ is —C(O)—; R¹⁷ is —CH₂(C₂H₄—O)_{y}, (C₂H₄—O)ₓ or is absent; and R¹⁸ is —C₂₋₄ alkyl. In some such embodiments, R¹⁵ is —C₂alkyl-NH—, x is 1 or 2, y is 1 and R¹⁸ is —C₂₋₄ alkyl.

In some embodiments, R¹⁴ is absent; R¹⁵ is absent; R¹⁶ is —C(O)—NH—, or is absent; R¹⁷ is —CH₂(C₂H₄—O)_{y}, (C₂H₄—O)ₓ, (C₃H₆—O)ₓ, or is absent; and R¹⁸ is —C₁₋₆ alkyl.

In some embodiments, at least one of R¹⁴-R¹⁸ is not —C₁₋₆ alkyl.

In some embodiments, x is 1, 2 or 3; y is 2 and R¹⁸ is —C₂₋₆ alkyl.

In some embodiments, when R¹⁵ is —C₁₋₆ alkyl-NH—, at least one of R¹⁶—R¹⁸ is present.

In some embodiments, when R¹⁷ is —CH₂(C₂H₄—O)_{y}, (C₂H₄—O)ₓ or (C₃H₆—O)ₓ, at least one of R¹⁴—R¹⁶ and R¹⁸ is present, wherein at least one of R¹⁴ and R¹⁸ is not —C₁₋₆ alkyl.

In some embodiments, [linker] is selected from

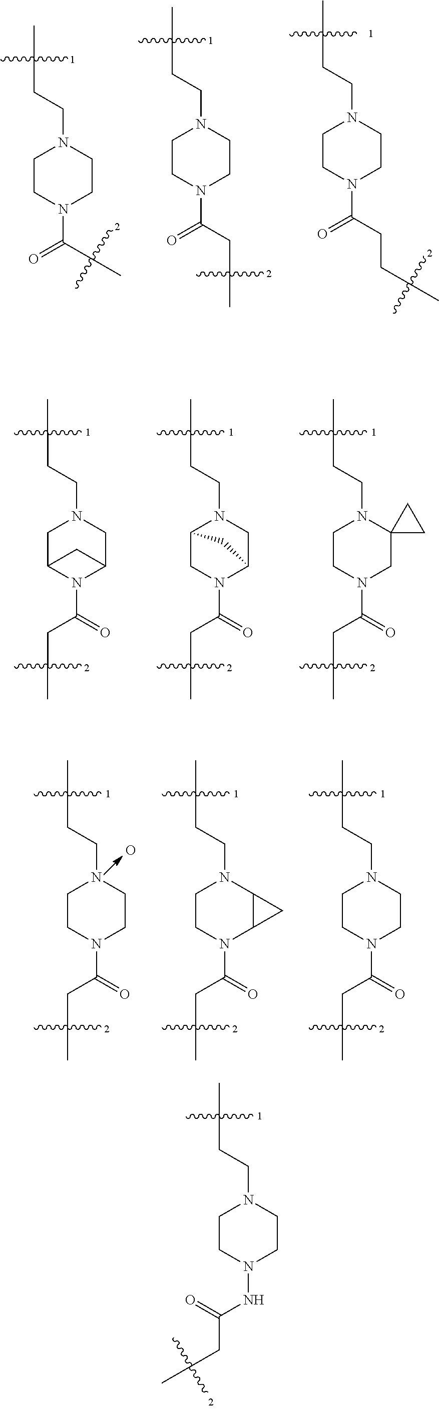

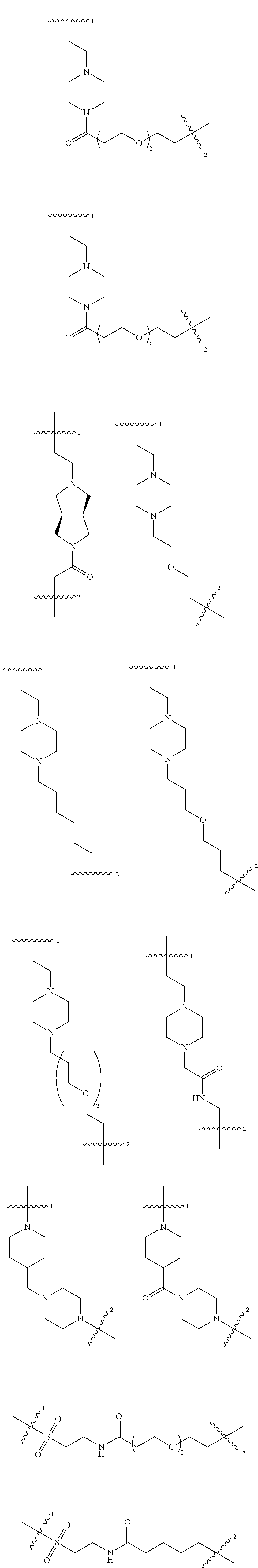

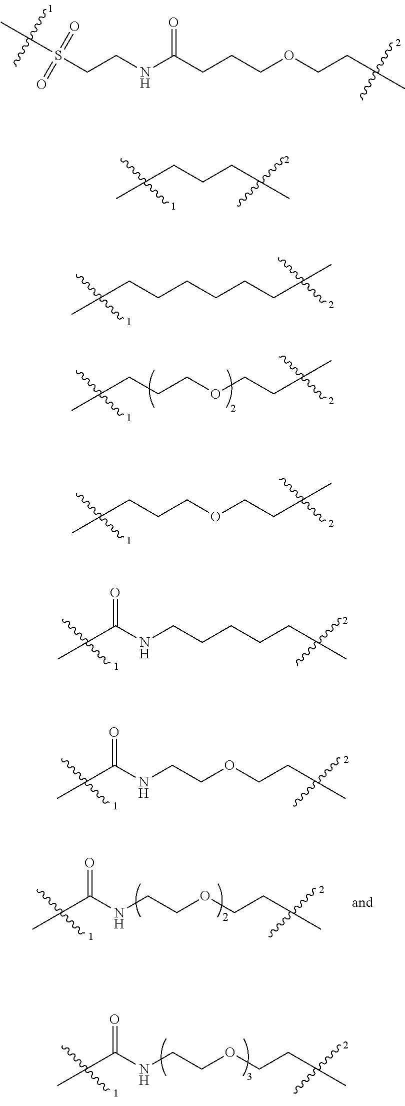

wherein
[symbol] indicates attachment to [MCL-1 ligand moiety] and
[symbol] indicates attachment to [ligase ligand moiety].

In some embodiments, [linker] is selected from

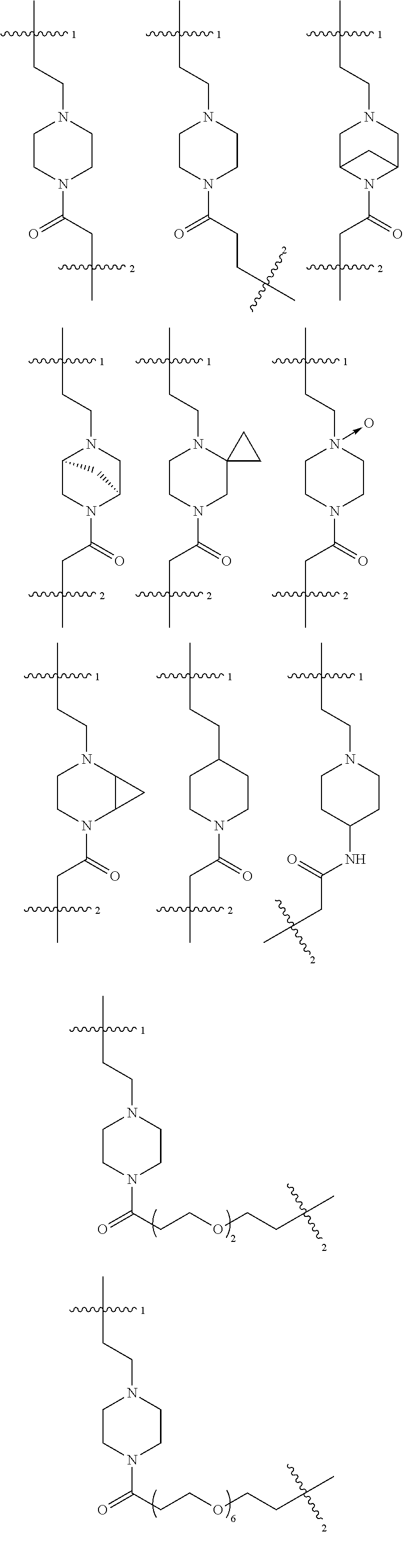

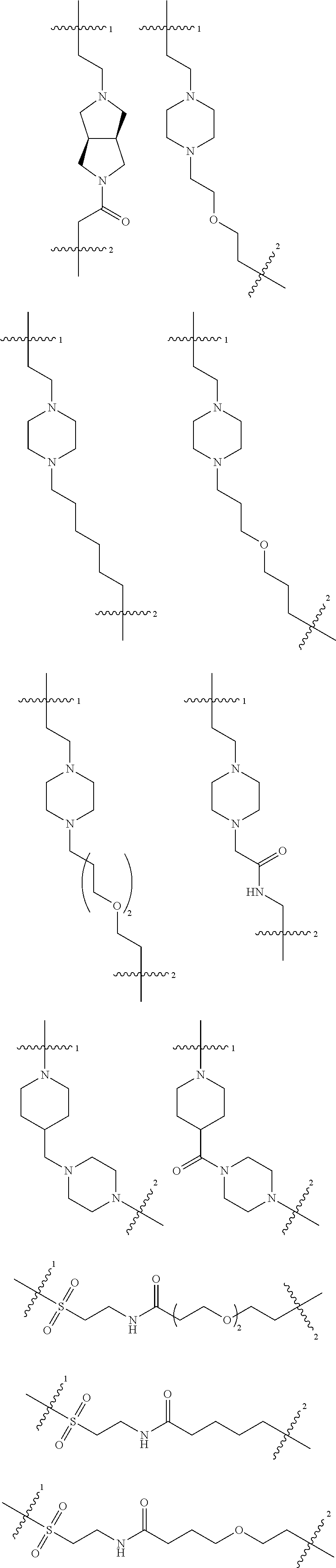

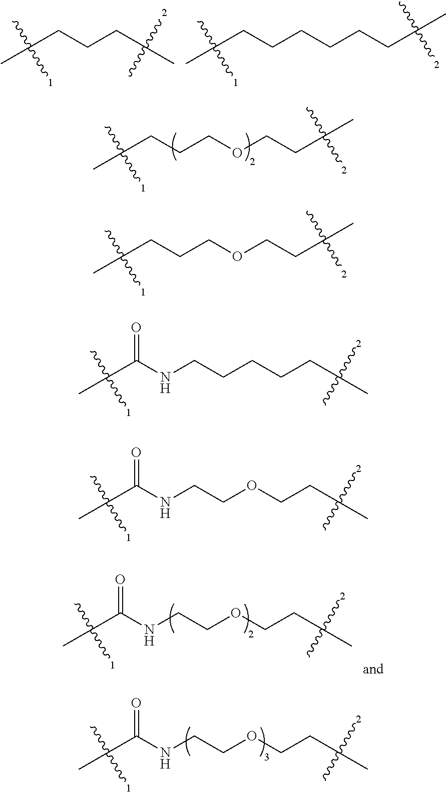

wherein
[symbol] indicates attachment to [MCL-1 ligand moiety] and
[symbol] indicates attachment to [ligase ligand moiety].

In some embodiments, [linker] is

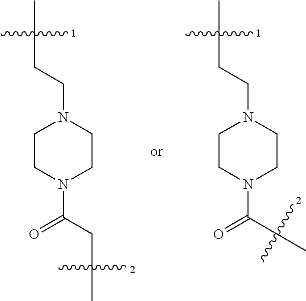 [SMILES: *CCN1CCN(C(=O)C*)CC1 ; *CCN1CCN(C(*)=O)CC1]

wherein
[symbol] indicates attachment to [MCL-1 ligand moiety] and
[symbol] indicates attachment to [ligase ligand moiety].

In some embodiments, R¹⁰ is —C₂₋₅alkyl-O—R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the naphthyl is optionally substituted with —O— or —S—.

In some embodiments, R¹² is H,

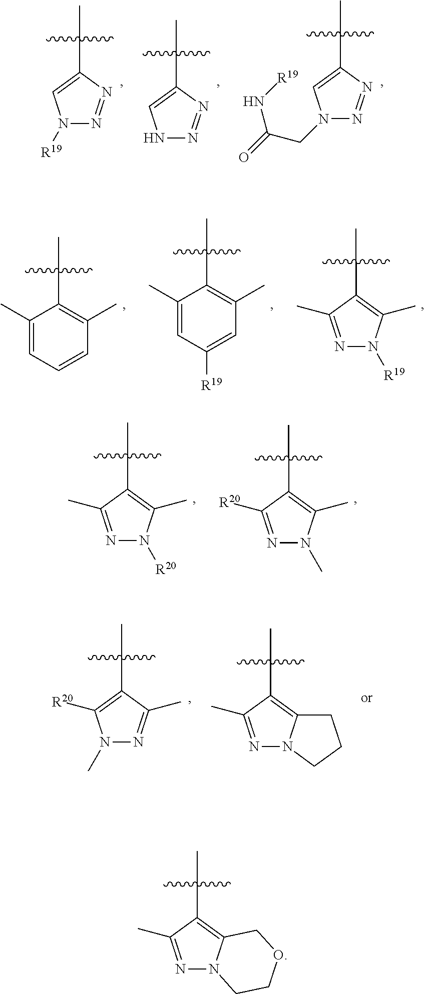

In some embodiments, R²⁰ is Me, —CH₂—O-bromobenzaldehyde, or

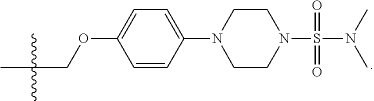 [SMILES: *COc1ccc(N2CCN(S(=O)(=O)N(C)C)CC2)cc1]

In some embodiments, when R⁸ is H, R¹³ is

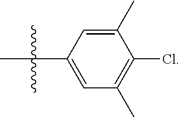 [SMILES: *c1cc(C)c(Cl)c(C)c1]

In some embodiments,
R⁸ is H, R¹⁹, methyl, or —CH₂CH₂-morpholine;
R⁹ is —C(O)OH or —C(O)NHR¹⁹,
R¹⁰ is —C₃H₆O—R¹³,
wherein R¹³ is

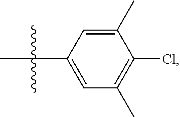 [SMILES: *c1cc(C)c(Cl)c(C)c1]

tetraline, or naphthyl optionally substituted with fluorine;
R¹¹ is H, Cl, F or methyl,
R¹² is

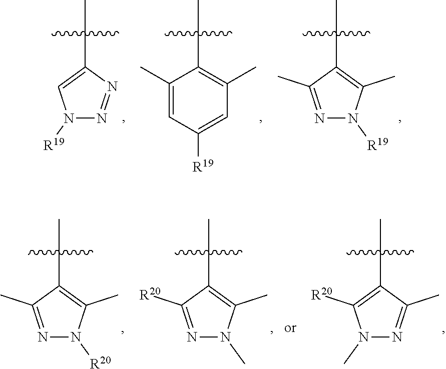

wherein R²⁰ is Me, —CH₂—O-bromobenzaldehyde, or

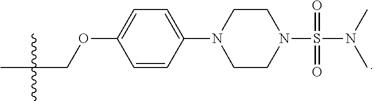 [SMILES: *COc1ccc(N2CCN(S(=O)(=O)N(C)C)CC2)cc1]

In some such embodiments, R⁸ is R¹⁹ or methyl; R¹⁰ is —C₃HO—R³, wherein R¹³ is naphthyl optionally substituted with fluorine; R¹¹ is Cl or F, and R¹² is

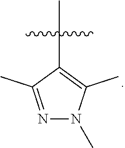

In some embodiments, Z₂ is C and

[symbol]

is a double bond.

In some embodiments, [MCL-1 ligand moiety] is

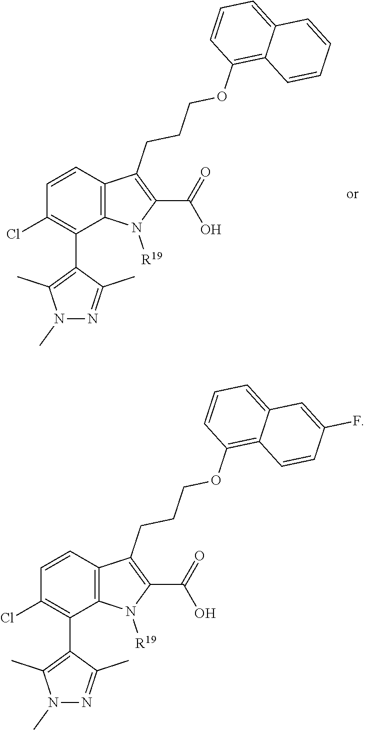

In some embodiments, the compound is selected from:

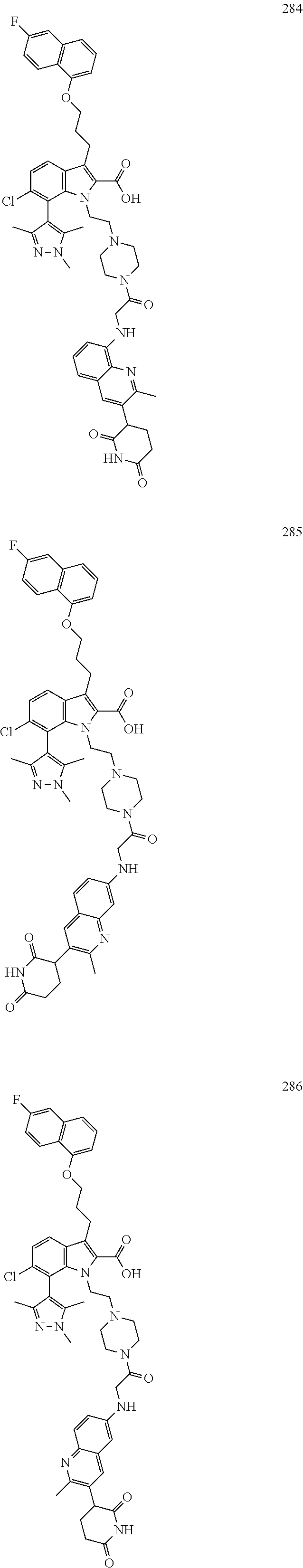

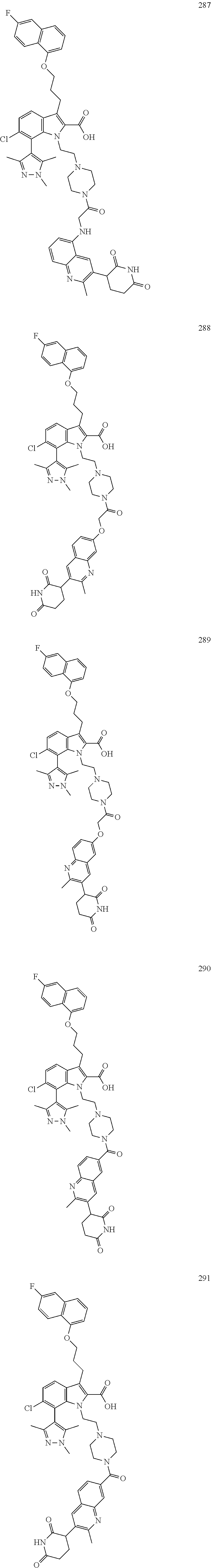

In some embodiments, the compound is:

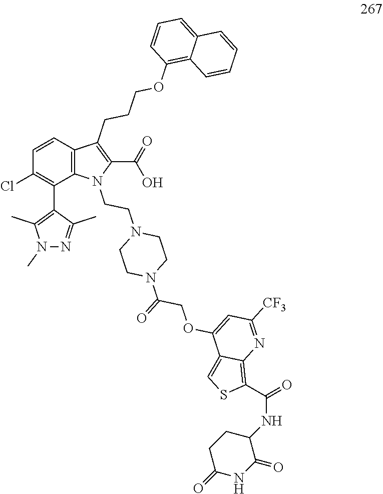 [SMILES: Cc1nn(C)c(C)c1-c1c(Cl)ccc2c(CCCOc3cccc4ccccc34)c(C(=O)O)n(CCN3CCN(C(=O)COc4cc(C(F)(F)F)nc5c(C(=O)NC6CCC(=O)NC6=O)scc45)CC3)c12]

In accordance with a third aspect of the invention, there is provided a compound of formula (I)

[MCL-11ligand moiety]-[linker]-[ligase ligand moiety] (I)

or a salt, solvate, hydrate, isomer or prodrug thereof,
wherein [ligase ligand moiety] is:
(a) Formula (II):

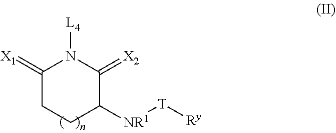

wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L₄ is hydrogen, alkyl, alkenyl, aryl, heteroaryl, benzyl, haloalkyl, haloalkenyl, —C(O)H, —C(O)R″, —C(O)OH, —C(O)OR″, —C(O)NH₂, —C(O)NHR″, —C(O)NR″₂, —OH, —OR″, —NH₂, —NHR″, —NR″₂, —S(O)₂H or —S(O)₂R″;
R^{y} is selected from

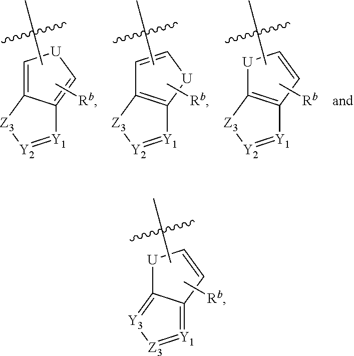

wherein [symbol] indicates attachment to T,
Z₃ is O, S or NR³;
U is O, S, NR^{b} or CR^{b}₂;
each of Y₁, Y₂ and Y₃ is independently N or CR^{d};
each R^{d} is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OH, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, —S(O)₂NR″₂, —O—R²¹, —NH—R²¹, —C(O)—NH—R²¹, or —CH₂—NH—C(O)—R²¹;
each R^{b} is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OH, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, or —S(O)₂NR″2;
each R³ is independently hydrogen, alkyl, cycloalkyl, alkenyl, cycloalkenyl, alkynyl, aryl, heteroaryl, benzyl, haloalkyl, haloalkenyl, —NH₂, —NHR″, —NR″₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OH, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, —S(O)₂NR″₂, —R²¹, —C(O)—NH—R²¹, or —CH₂—NH—C(O)—R²¹;
each R″ is independently alkyl, cycloalkyl, alkenyl, cycloalkenyl, aryl, heteroaryl, or benzyl;
R²¹ is a bond connected to R¹⁸ of the linker, wherein Formula (II) contains a single R²¹;
wherein,
(i) when R^{y} is

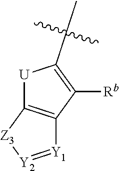

then Y₂ is CR^{d}; and
(ii) when R^{y} is

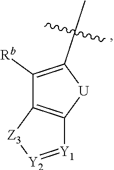

then R^{b} in CR^{b}₂ is not hydrogen or
(b) Formula (III):

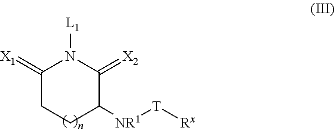

wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L₁ is hydrogen, alkenyl, aryl, heteroaryl, benzyl, haloalkyl, haloalkenyl, —C(O)H, —C(O)R″, —C(O)OH, —C(O)OR″, —CH₂C(O)OR″, —C(O)NH₂, —C(O)NHR″, —C(O)NR″₂, —OH, —OR″, —NH₂, —NHR″, —NR″₂, —S(O)₂H or —S(O)₂R′;
R^{x} is selected from

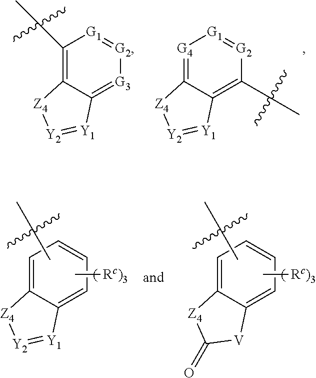

wherein [symbol] indicates attachment to T,
Z₄ is O, S or NR⁴;
V is CR^{f}₂, NR⁴ or S;
each of G₁, G₂, G₃ and G₄ is independently N or CR^{c},
each of Y₁ and Y₂ is independently N or CR^{f},
each R^{f} is independently hydrogen, halogen, alkyl, cycloalkyl, alkenyl, cycloalkenyl, alkynyl, aryl, fused aryl-cycloalkyl, fused aryl-heterocycloalkyl, heteroaryl, heteroaryl substituted with at least one aryl group, benzyl, haloalkyl, haloalkenyl, —NH₂, —NHR″, —NR″₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OH, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, —S(O)₂NR″₂, —R²¹, —O—R²¹, —NH—R²¹, —C(O)—NH—R²¹, or —CH₂—NH—C(O)—R²¹; or when Y₁ and Y₂ are CR^{f} then each R^{f}, together with the carbon atom to which it is attached, forms a 5- or 6-membered ring;
each R^{c} is independently hydrogen, halogen, alkyl, cycloalkyl, alkenyl, cycloalkenyl, alkynyl, aryl, aryl substituted with at least one —OR″, heteroaryl, benzyl, haloalkyl, haloalkenyl, —NH₂, —NHR″, —NR″₂, —CH₂NH₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, —S(O)₂NR″₂, —O—R²¹, —NH—R²¹, —C(O)—NH—R²¹, or —CH₂—NH—C(O)—R²¹;
each R⁴ is independently hydrogen, halogen, alkyl, cycloalkyl, alkenyl, cycloalkenyl, alkynyl, aryl, heteroaryl, benzyl, haloalkyl, haloalkenyl, —C(O)H, C(O)R″, —C(O)OH, —C(O)OR″, —C(O)NH₂, —C(O)NHR″, —C(O)NR″₂, —OH, —OR″, —NH₂, —NHR″, —NR″₂, —S(O)₂H, —S(O)₂R″, —R²¹, —C(O)—NH—R²¹, or —CH₂—NH—C(O)—R²¹; and
each R″ is independently alkyl, cycloalkyl, alkenyl, cycloalkenyl, aryl, heteroaryl, or benzyl;
R²¹ is a bond connected to R¹⁸ of the linker, wherein Formula (III) contains a single R²¹;
wherein, when n=2, each R^{c} is hydrogen, and each of G₁, G₂, G₃ and G₄ is CR^{c}, then C═X₁ may be replaced by CH;
   and wherein:
(i) when R^{x} is

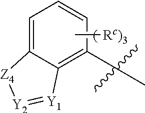

and Z₄ is NH, then L₁ is hydrogen, —CH₂C(O)OR″, or —OR″;
(ii) when R^{x} is

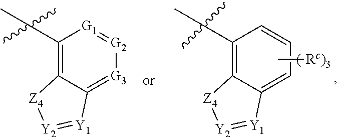

Z₄ is NR⁴, Y₁ is CR^{f}, and Y₂ is N, then R⁴ is not alkyl and at least one of R² and R is not H;
(iii) when R^{x} is

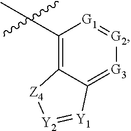

Z₄ is NR⁴, and Y₁ and Y₂ are CR^{f}, then at least one of G₁, G₂ and G₃ is N;
(iv) when Z₄ is NR⁴, and Y₁ and Y₂ are CR^{f}, then R^{x} is not

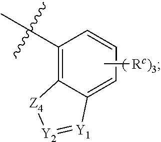

(v) when R^{x} is

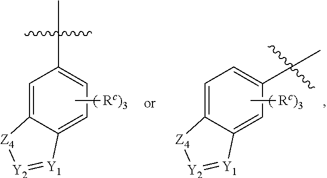

Z₄ is NR⁴, and Y₁ or Y₂ is N, then R⁴ is not alkyl;
(vi) when R^{x} is

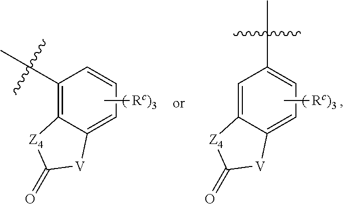

then n=1 or 2; and
(vii) when R^{x} is

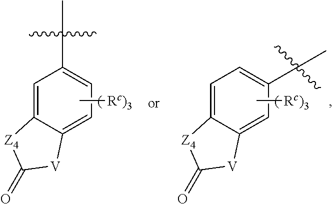

then Z₄═O or S
   wherein
   [MCL-1 ligand moiety] is a compound of Formula (A), Formula (B) or Formula (C)

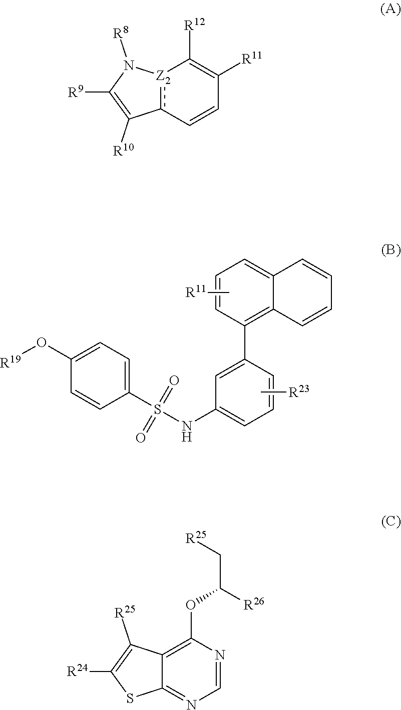

wherein
[symbol] is a single bond or a double bond;
R⁸ is H, R¹⁹, or C₁-C₆ alkyl optionally substituted with morpholine;
R⁹ is —C(O)OH, —C(O)OC₁-C₆alkyl; —C(O)NH₂; —C(O)OR¹⁹ or —C(O)NHR¹⁹,
R¹⁰ is —C₂₋₅alkyl-O—R¹³ or —C₂₋₅alkyl-NMe-R¹³ wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹¹ is H, halogen or C₁-C₆ alkyl,
R¹² is H,

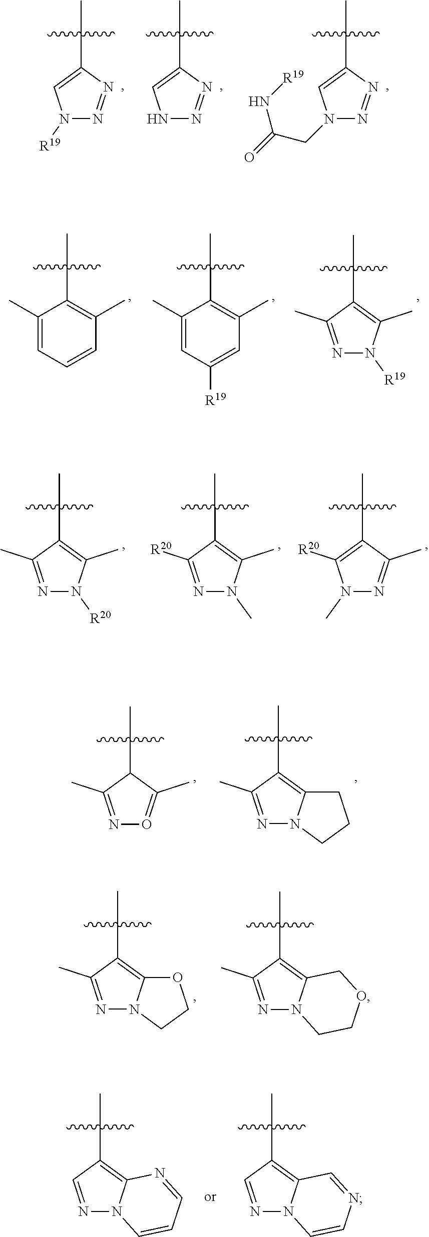

wherein R²⁰ is Me, —CH₂—OMe, —CH₂—O-bromobenzaldehyde, or

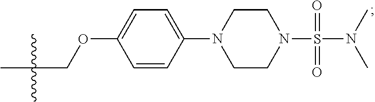 [SMILES: *COc1ccc(N2CCN(S(=O)(=O)N(C)C)CC2)cc1]

or when R¹² is

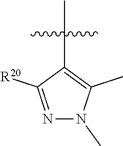

and R¹⁰ is —O-naphthyl substituted with —O— or —S—, then R²⁰ is

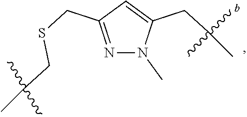 [SMILES: *CSCc1cc(C*)n(C)n1]

wherein [symbol] indicates attachment to —O— or —S— of R¹⁰;
and wherein
R¹⁹ is a bond connected to R¹⁴ of the linker;
R²³ is —C(O)OH or —C(O)OC₁-C₆ alkyl;
Z₂ is N or C, wherein when Z₂ is N, then [symbol] is a single bond; and when Z₂ is C, then [symbol] is a double bond,
R²⁴ is furan optionally substituted with at least one halogen,
each R²⁵ is independently phenyl substituted with —OR²⁸ and optionally further substituted with at least one substituent selected from halogen and C₁-C₆ alkyl;
R²⁶ is —C(O)OR¹⁹ or —C(O)NHR¹⁹; and
each R²⁸ is independently —C₁₋₃alkyl-(N-alkyl piperazine) or —C₁₋₃alkyl-(N-haloalkylpyrazole)
and wherein each of Formula (A), Formula (B) and Formula (C) contains a single R¹⁹;
   and wherein [linker] has the following formula

R¹⁴—R¹⁵—R¹⁶—R¹⁷—R¹⁸

wherein
R¹⁴ is —C₁₋₆alkyl, —C₂₋₆alkenyl, —C₂₋₆alkynyl, C₁₋₆ alkyl-N(C₁₋₆ alkyl)-, —C(O)—, —SO₂— or is absent
R¹⁵ is cycloalkyl, heterocycloalkyl, aryl, heteroaryl, —C₁₋₆ alkyl-NH—, C₁₋₆ alkyl-N(C₁₋₆ alkyl)-, -cycloalkyl-NH—, heterocycloalkyl-NH—, or is absent
R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, (C₂H₄—O)ₓ, (C₃H₆—O)ₓ, or is absent
x is 1-10
y is 2-10
R¹⁸ is —C₁₋₆ alkyl, heterocycloalkyl, or is absent
wherein at least one of R¹⁴-R¹⁸ is present

In some embodiments, each alkyl, alkenyl, alkynyl, aryl, heteroaryl and benzyl is unsubstituted.

In some embodiments, in Formula (III): each of X₁ and X₂ is O; T is C═O; R¹ is hydrogen, L₁ is hydrogen, R^{x} is

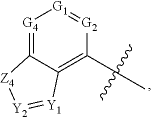

Z₄ is NR⁴; each of G₁, G₂ and G₄ is CR^{c}, Y₁ is N, and Y₂ is CR^{f}, wherein R^{f} is not hydrogen.

In some embodiments, [ligase ligand moiety] is Formula (III):

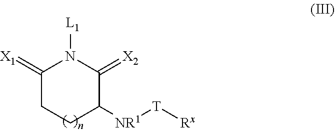

In some embodiments, one of R is —O—R²¹, —NH—R²¹, —C(O)—NH—R²¹, or —CH₂—NH—C(O)—R²¹.

In some embodiments, G₁ is C—O—R²¹, C—NH—R²¹, C—C(O)—NH—R²¹, or C—CH₂—NH—C(O)—R²¹. In other embodiments, G₂ is C—O—R²¹, C—NH—R²¹, C—C(O)—NH—R²¹, or C—CH₂—NH—C(O)—R²¹.

In some embodiments, R⁴ is R²¹, —C(O)—NH—R²¹, or —CH₂—NH—C(O)—R²¹.

In some embodiments, one of R^{f} is —R²¹, —O—R²¹, —NH—R²¹, —C(O)—NH—R²¹, or —CH₂—NH—C(O)—R²¹.

In some embodiments, Y₂ is C—R²¹, CO—R²¹, C—NH—R²¹, C—C(O)—NH—R²¹, or C—CH₂—NH—C(O)—R²¹.

In some embodiments, [ligase ligand moiety] is selected from

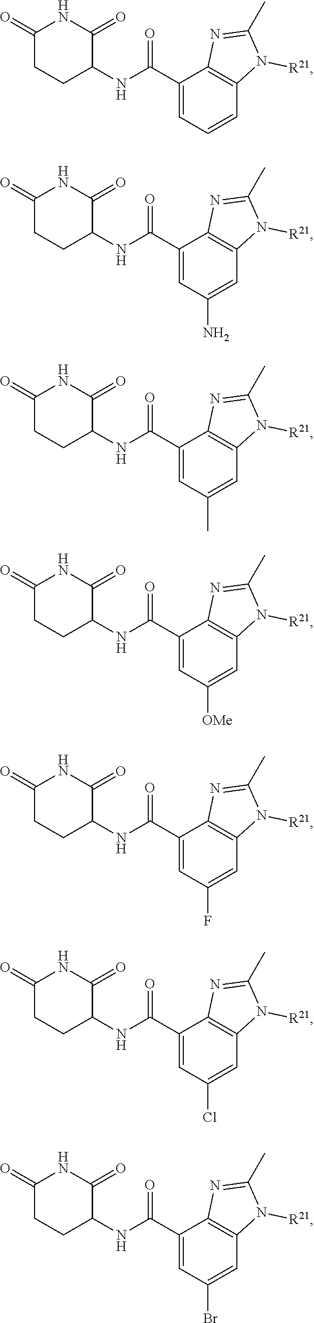

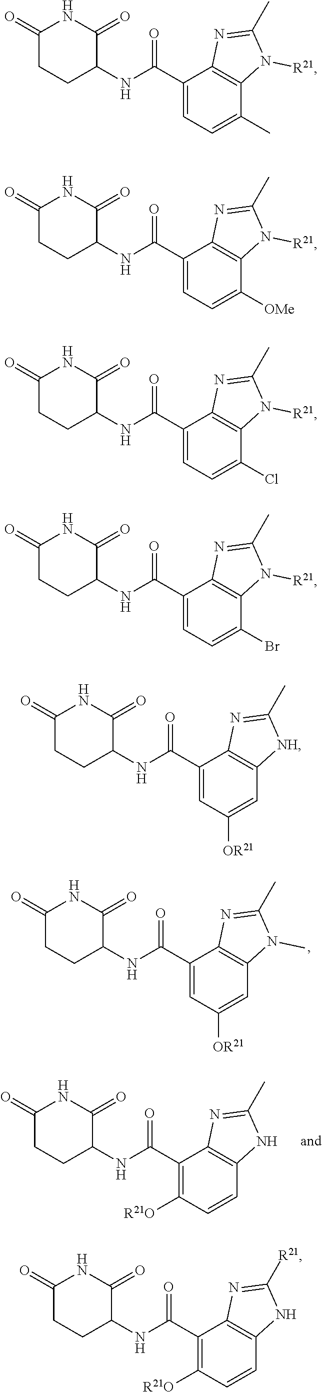

In some embodiments, [ligase ligand moiety] is selected from

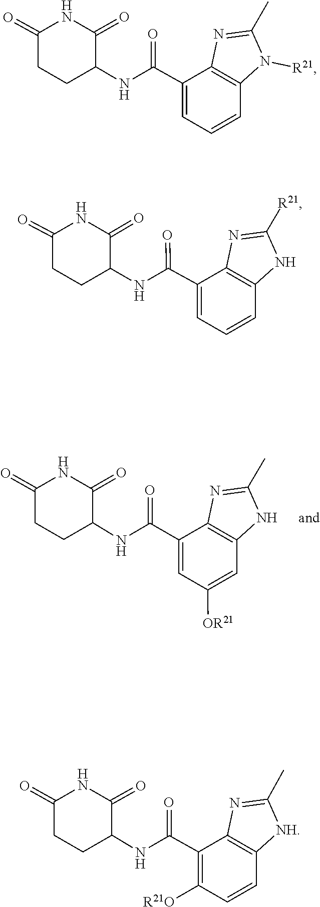

In some embodiments, [ligase ligand moiety] is of Formula (II):

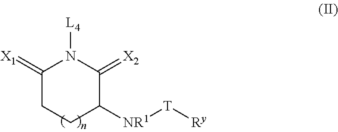

In some embodiments, R^{y} is selected from

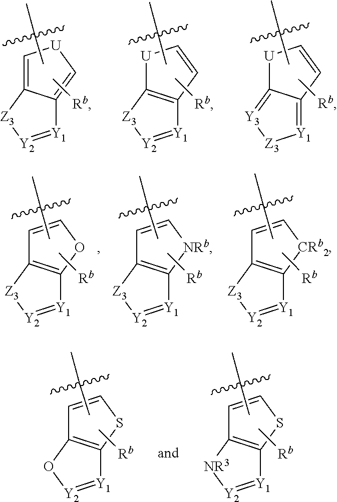

In some embodiments, Z₃ is S or NR³; U is O or S; and each of Y₁, Y₂ and Y₃ is independently N or CR^{d}.

In some embodiments, R^{b} is hydrogen or alkyl.

In some embodiments, R³ is hydrogen, alkyl, cycloalkyl, —R²¹, —C(O)—NH—R²¹, or —CH₂—NH—C(O)—R²¹.

In some embodiments, each R^{d} is independently hydrogen, alkyl, —O—R²¹, —NH—R²¹, —C(O)—NH—R²¹, or —CH₂—NH—C(O)—R²¹.

In some embodiments, R¹⁴ is —C₁₋₆ alkyl, —C₂₋₆ alkenyl, —C₂₋₆ alkynyl, —C(O)—, —SO₂— or is absent.

In some embodiments, R¹⁵ is cycloalkyl, heterocycloalkyl, aryl, heteroaryl, —C₁₋₆ alkyl-NH—, -cycloalkyl-NH— or is absent.

In some embodiments,
R¹⁴ is —C₁₋₆ alkyl, —SO₂— or is absent
R¹⁵ is piperazine, bridged piperazine, piperazine N-oxide, piperazine cation, —C₁₋₆ alkyl-NH—,

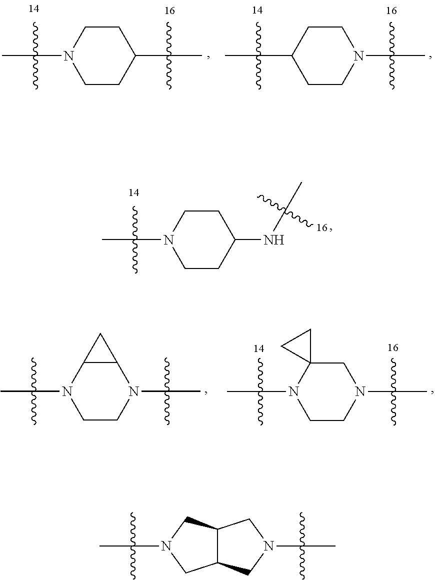 [SMILES: *C1CCN(*)CC1 ; *C1CCN(*)CC1 ; *NC1CCN(*)CC1 ; *N1CCN(*)C2CC21 ; *N1CCN(*)C2(CC2)C1 ; *N1C[C@H]2CN(*)C[C@H]2C1]

or is absent, wherein [symbol] indicates attachment to R¹⁴ and [symbol] indicates attachment to R¹⁶,
R¹⁶ is —C₁₋₆ alkyl, —C(O)—, —C(O)—NH—, —CH₂—C(O)—NH— or is absent
R¹⁷ is —CH₂(C₂H₄—O)_{y}, (C₂H₄—O)ₓ, (C₃H₆—O)ₓ, or is absent
x is 1-6
y is 2-6
R¹⁸ is —C₁₋₆ alkyl, piperazine, or is absent
   wherein at least one of R¹⁴-R¹⁸ is present.

In some embodiments, R¹⁸ is —C₁₋₆ alkyl or is absent.

In some embodiments, when R¹⁵ is piperazine, bridged piperazine, piperazine N-oxide, piperazine cation, —C₁₋₆ alkyl-NH—,

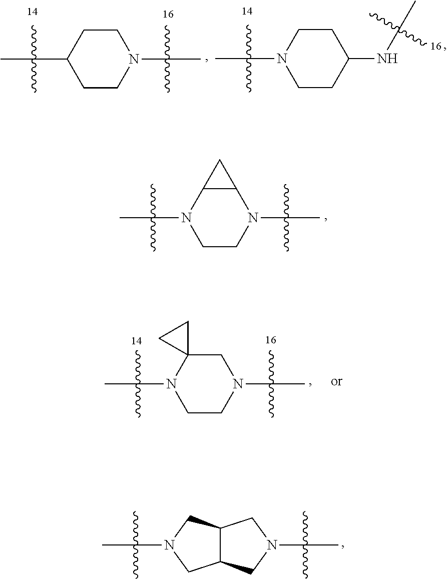 [SMILES: *C1CCN(*)CC1 ; *NC1CCN(*)CC1 ; *N1CCN(*)C2CC21 ; *N1CCN(*)C2(CC2)C1 ; *N1C[C@H]2CN(*)C[C@H]2C1]

then R¹⁴ is —C₁₋₆ alkyl.

In some embodiments, R¹⁴ is —C₁₋₆ alkyl,
R¹⁵ is piperazine, bridged piperazine, piperazine N-oxide,

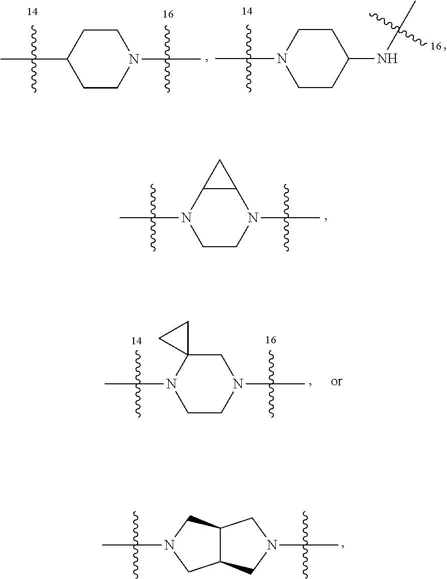 [SMILES: *C1CCN(*)CC1 ; *NC1CCN(*)CC1 ; *N1CCN(*)C2CC21 ; *N1CCN(*)C2(CC2)C1 ; *N1C[C@H]2CN(*)C[C@H]2C1]

R¹⁶ is —C(O)—, —CH₂—C(O)—NH—, or is absent
R¹⁷ is —CH₂(C₂H₄—O)_{y}, (C₂H₄—O)ₓ, (C₃H₆—O)ₓ, or is absent
R¹⁸ is —C₁₋₆ alkyl.
   wherein when R¹⁶ and R¹⁷ are absent, R¹⁸ is —C₃₋₆ alkyl. In some such embodiments, R¹⁴ is —C₂ alkyl; x is 1, 2 or 6; and y is 2. In other such embodiments, R¹⁵ is piperazine, R¹⁶ is —C(O)—, and R¹⁷ is - absent. In some such embodiments, R¹⁴ is —C₂ alkyl, and R¹⁸ is —C₁₋₂ alkyl.

In some embodiments, when R¹⁴ is —SO₂—, at least two of R¹⁵-R¹⁸ are present, and at least one of R¹⁵—R¹⁸ is not C₁₋₆ alkyl.

In some embodiments, R¹⁴ is —SO₂—; R¹⁵ is —C₁₋₆ alkyl-NH—; R¹⁶ is —C(O)—; R¹⁷ is —CH₂(C₂H₄—O)_{y}, (C₂H₄—O)ₓ or is absent; and R¹⁸ is —C₂₋₄ alkyl. In some such embodiments, R¹⁵ is —C₂alkyl-NH—; x is 1 or 2; y is 1; and R¹⁸ is —C₂₋₄ alkyl

In some embodiments, R¹⁴ is absent; R¹⁵ is absent; R¹⁶ is —C(O)—NH—, or is absent; R¹⁷ is —CH₂(C₂H₄—O)_{y}, (C₂H₄—O)ₓ, (C₃H₆—O)ₓ, or is absent; and R¹⁸ is —C₁₋₆ alkyl.

In some embodiments, at least one of R¹⁴-R¹⁸ is not —C₁₋₆ alkyl.

In some embodiments, x is 1, 2 or 3; y is 2; and R¹⁸ is —C₂₋₆ alkyl.

In some embodiments, R¹⁵ is —C₁₋₆ alkyl-NH—, at least one of R¹⁶—R¹⁸ is present.

In some embodiments, when R¹⁷ is —CH₂(C₂H₄—O)_{y}, (C₂H₄—O)ₓ or (C₃H₆—O)ₓ, at least one of R¹⁴—R¹⁶ and R¹⁸ is present, wherein at least one of R¹⁴ and R¹⁸ is not —C₁₋₆ alkyl.

In some embodiments, [linker] is selected from

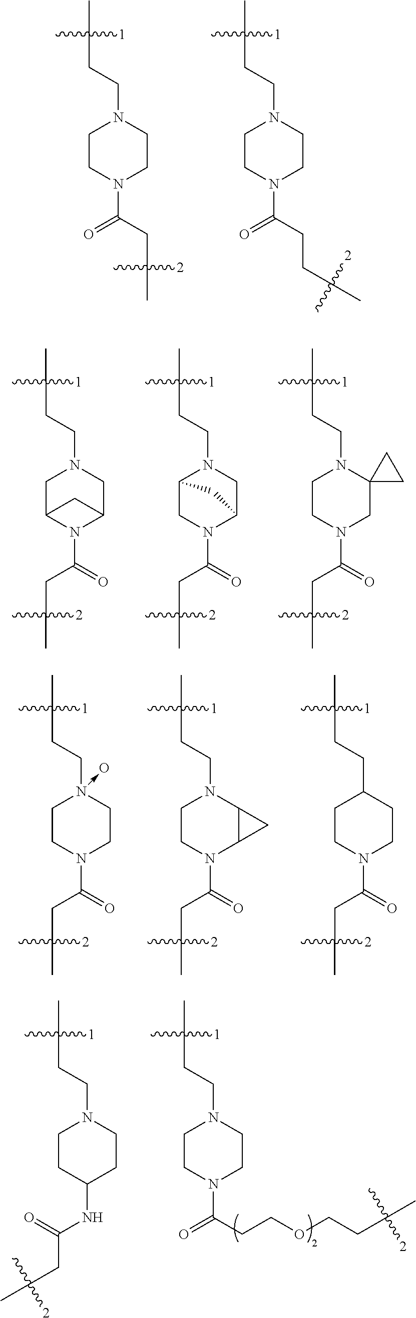

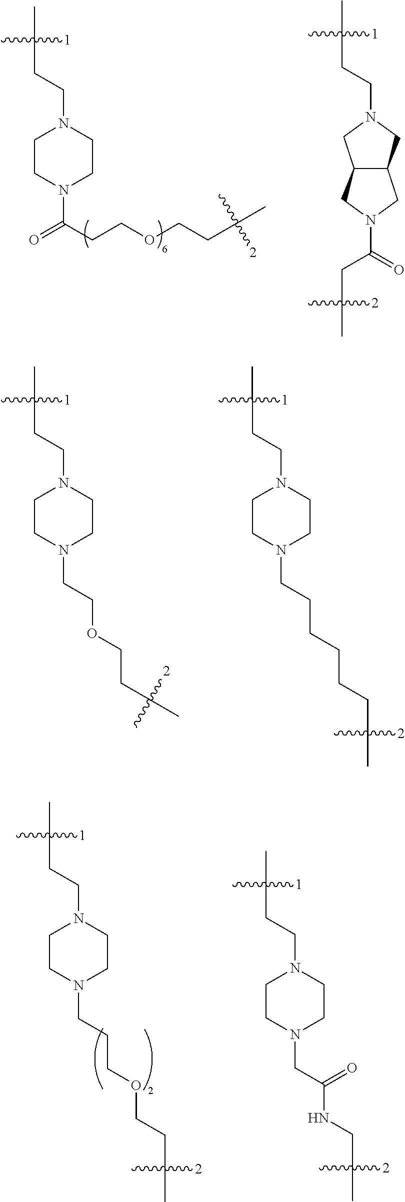

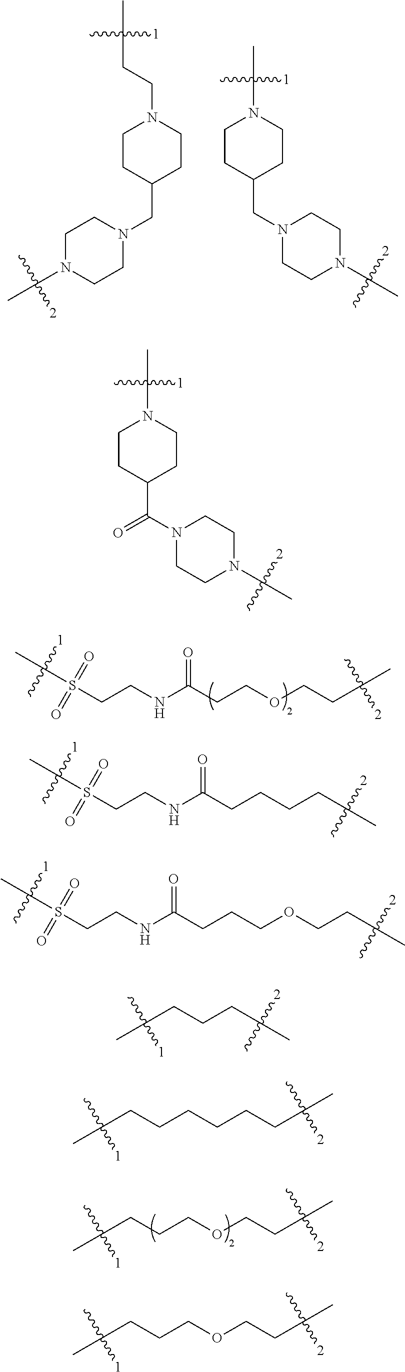

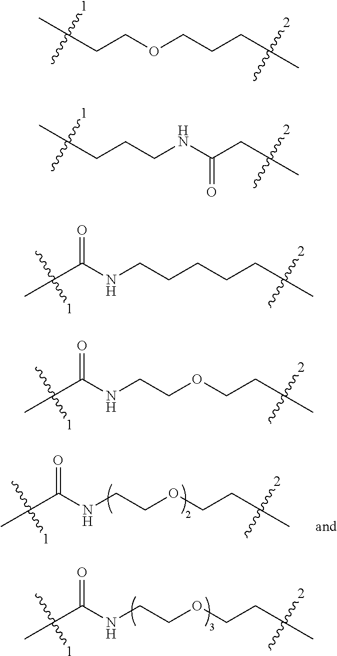

wherein
[symbol] indicates attachment to [MCL-1 ligand moiety] and
[symbol] indicates attachment to [ligase ligand moiety].

In some embodiments, [linker] is selected from

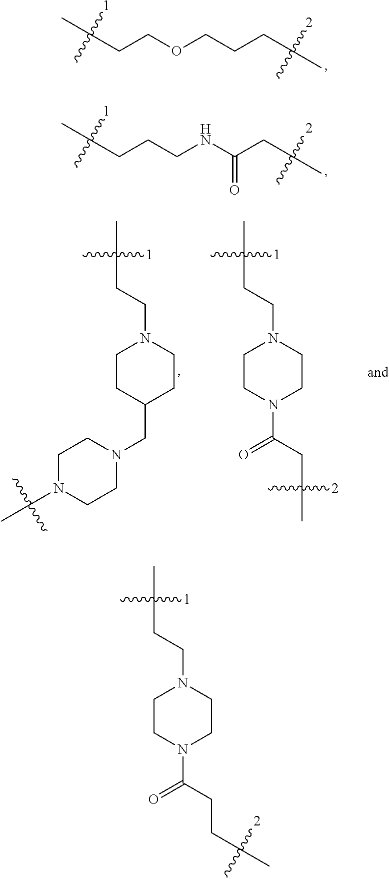

wherein
[symbol] indicates attachment to [MCL-1 ligand moiety] and
[symbol] indicates attachment to [ligase ligand moiety].

In some embodiments, R¹⁰ is —C₂₋₅alkyl-O—R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the naphthyl is optionally substituted with —O— or —S—.

In some embodiments, R¹² is H,

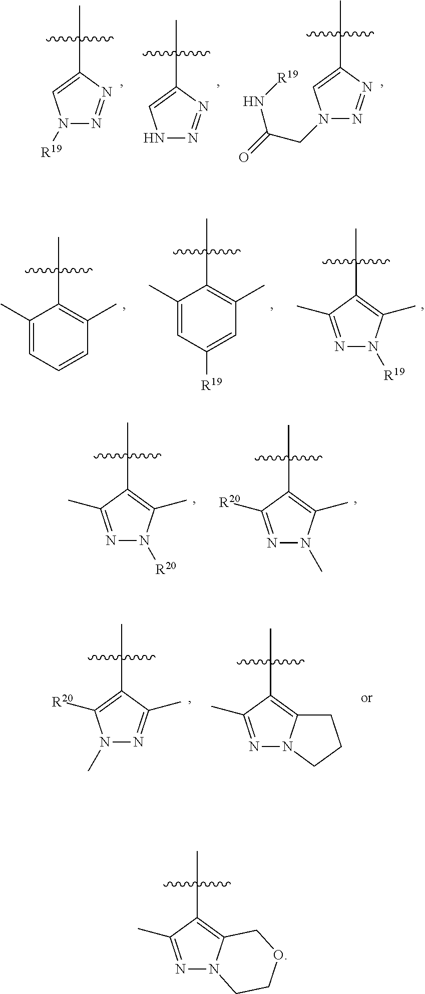

In some embodiments, R²⁰ is Me, —CH₂—O-bromobenzaldehyde, or

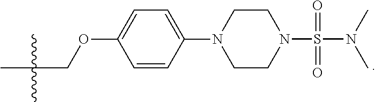 [SMILES: *COc1ccc(N2CCN(S(=O)(=O)N(C)C)CC2)cc1]

In some embodiments, when R⁸ is H, R¹³ is

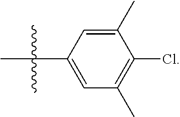 [SMILES: *c1cc(C)c(Cl)c(C)c1]

In some embodiments,
R⁸ is H, R¹⁹, methyl, or —CH₂CH₂-morpholine;
R⁹ is —C(O)OH or —C(O)NHR¹⁹,
R¹⁰ is —C₃H₆O—R¹³,
wherein R¹³ is

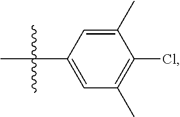 [SMILES: *c1cc(C)c(Cl)c(C)c1]

tetraline or naphthyl optionally substituted with fluorine;
R¹¹ is H, Cl, F or methyl,
R¹² is

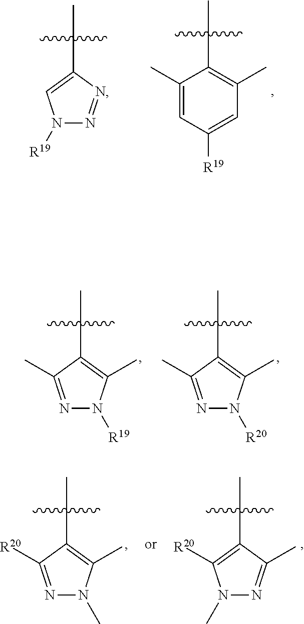

wherein R²⁰ is Me, —CH₂—O-bromobenzaldehyde, or

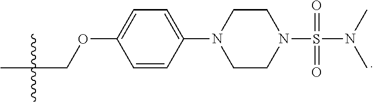 [SMILES: *COc1ccc(N2CCN(S(=O)(=O)N(C)C)CC2)cc1]

In some such embodiments, R⁸ is R¹⁹ or methyl; R¹⁰ is —C₃HO—R¹³, wherein R¹³ is naphthyl optionally substituted with fluorine; R¹¹ is Cl or F, and R¹² is

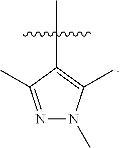

In some embodiments, Z₂ is C and

[symbol]

is a double bond.

In some embodiments, [MCL-1 ligand moiety] is

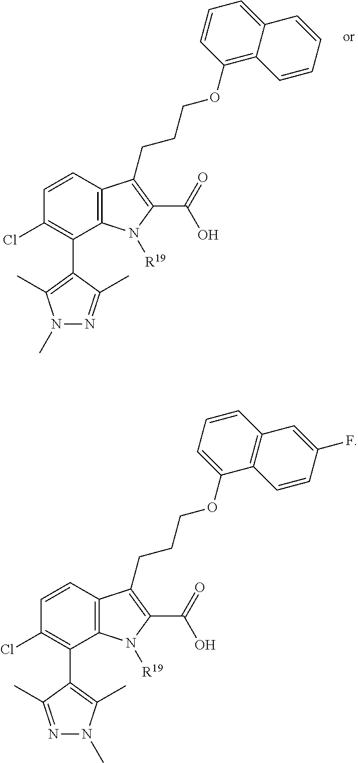

In some embodiments, the compound is selected from

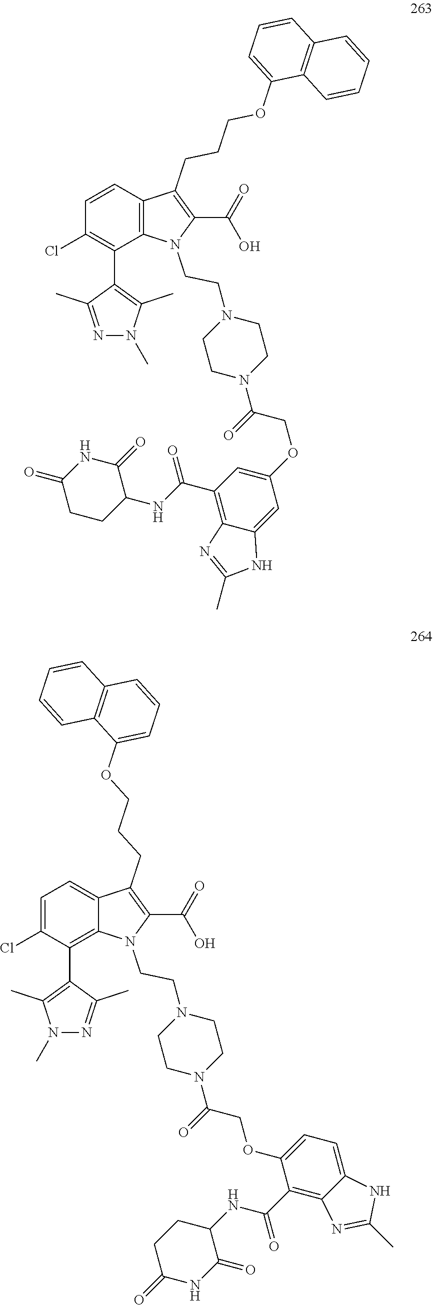

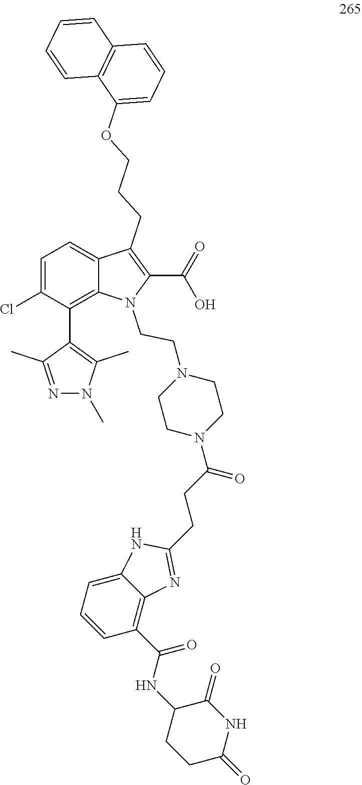 [SMILES: Cc1nn(C)c(C)c1-c1c(Cl)ccc2c(CCCOc3cccc4ccccc34)c(C(=O)O)n(CCN3CCN(C(=O)CCc4nc5c(C(=O)NC6CCC(=O)NC6=O)cccc5[nH]4)CC3)c12]

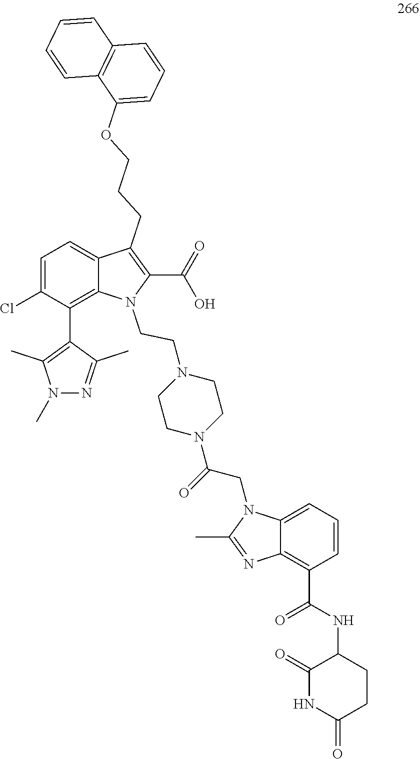 [SMILES: Cc1nn(C)c(C)c1-c1c(Cl)ccc2c(CCCOc3cccc4ccccc34)c(C(=O)O)n(CCN3CCN(C(=O)Cn4c(C)nc5c(C(=O)NC6CCC(=O)NC6=O)cccc54)CC3)c12]

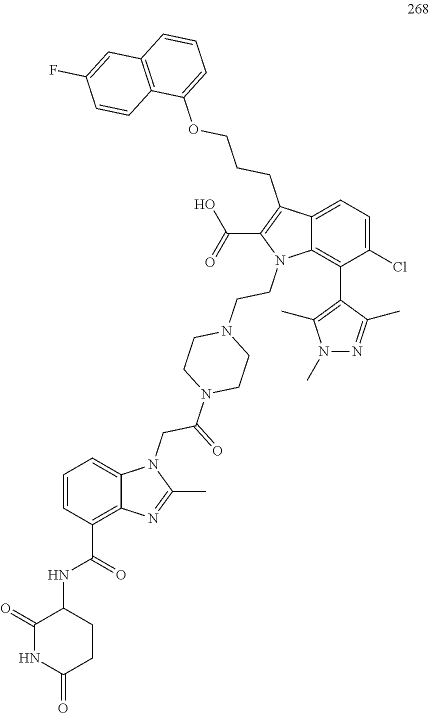 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cccc54)CC3)c12]

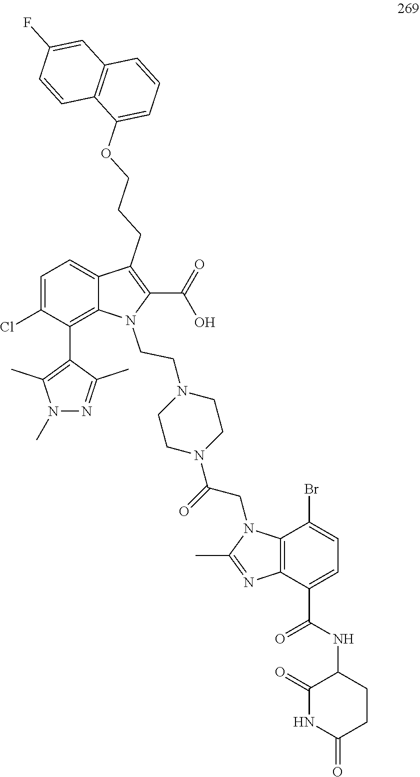 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)ccc(Br)c54)CC3)c12]

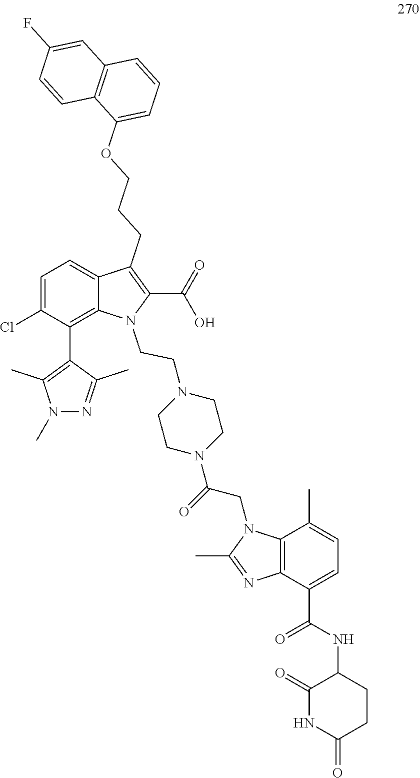 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)ccc(C)c54)CC3)c12]

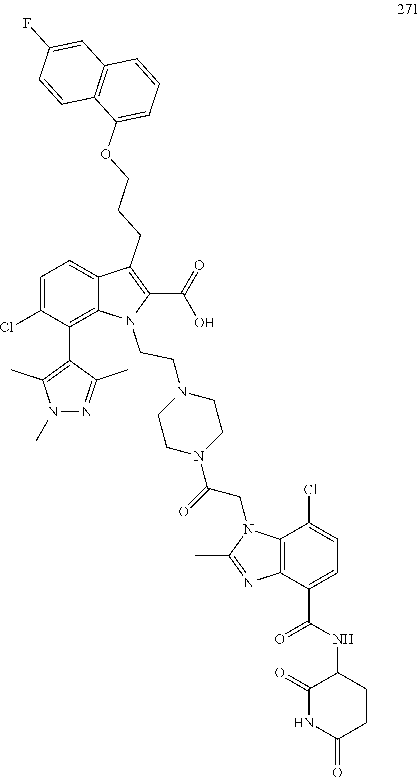 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)ccc(Cl)c54)CC3)c12]

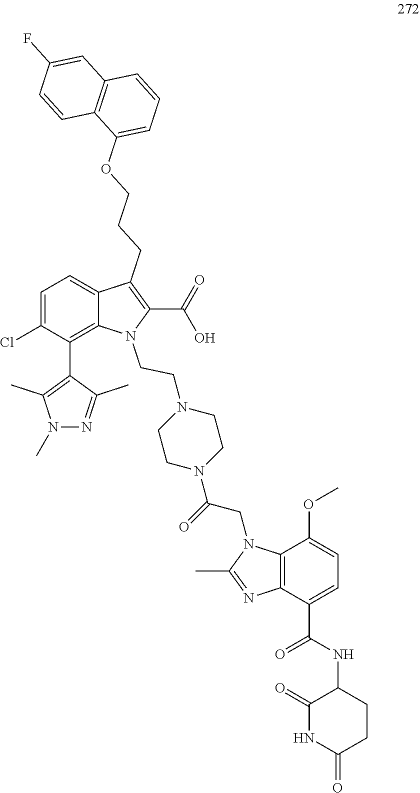 [SMILES: COc1ccc(C(=O)NC2CCC(=O)NC2=O)c2nc(C)n(CC(=O)N3CCN(CCn4c(C(=O)O)c(CCCOc5cccc6cc(F)ccc56)c5ccc(Cl)c(-c6c(C)nn(C)c6C)c54)CC3)c12]

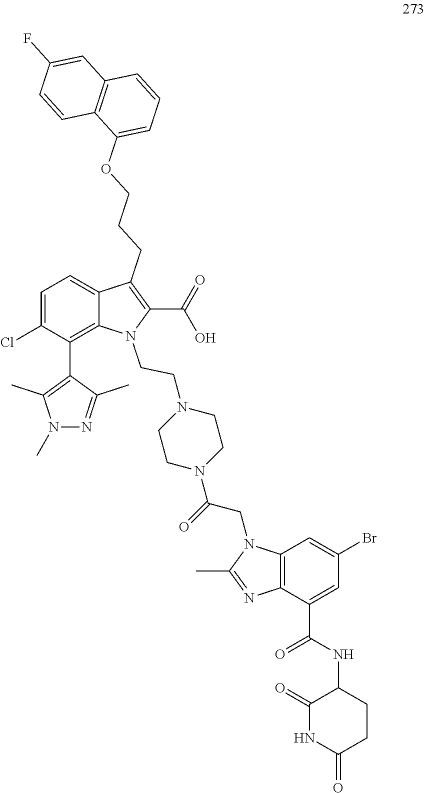 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cc(Br)cc54)CC3)c12]

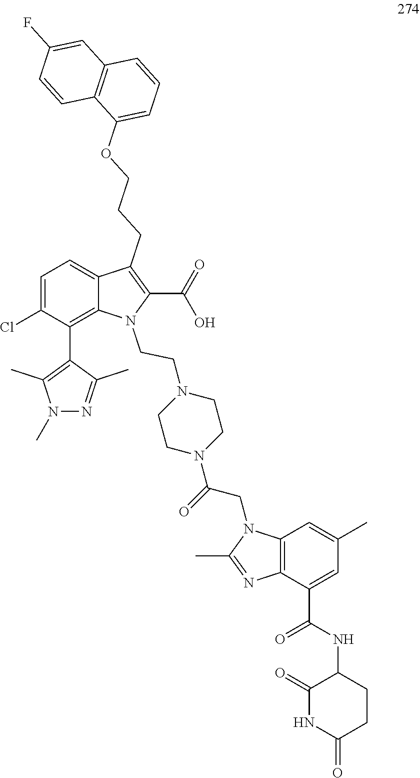

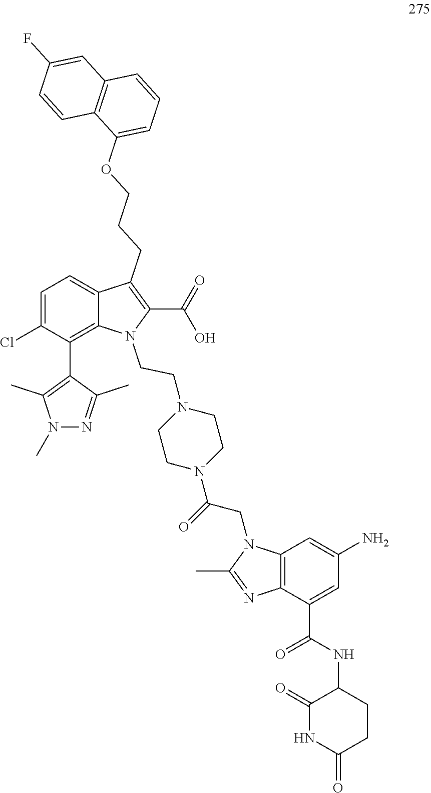 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cc(N)cc54)CC3)c12]

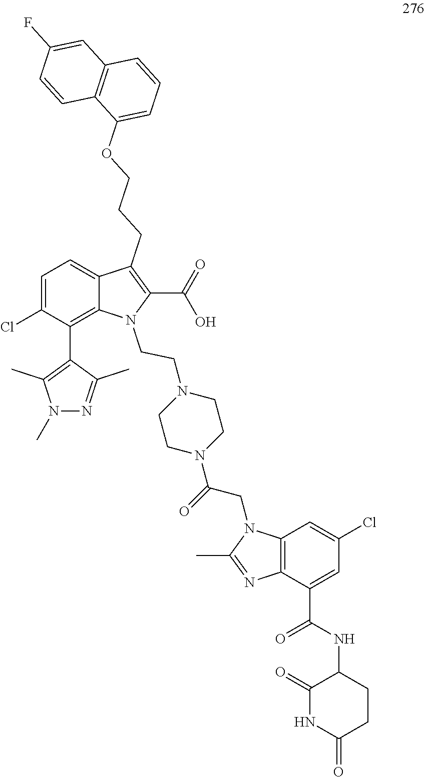 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cc(Cl)cc54)CC3)c12]

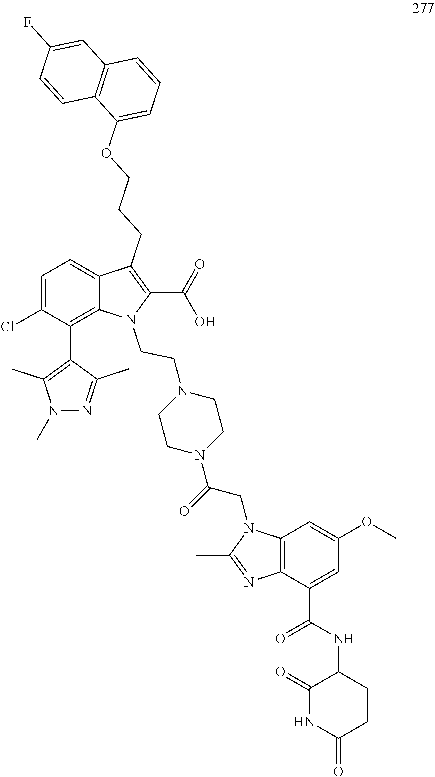

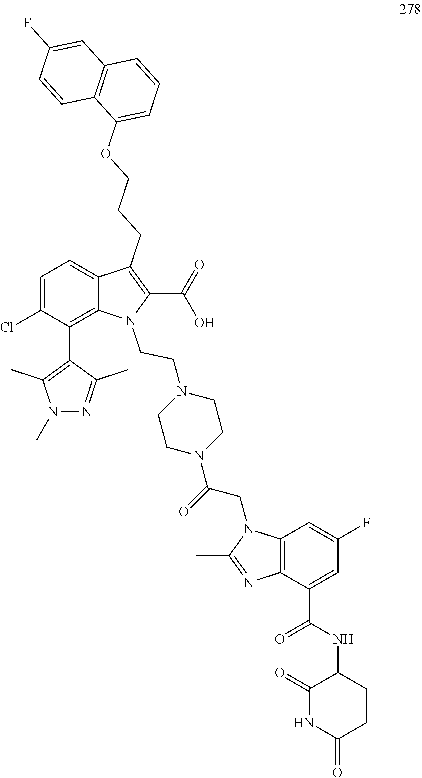 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cc(F)cc54)CC3)c12]

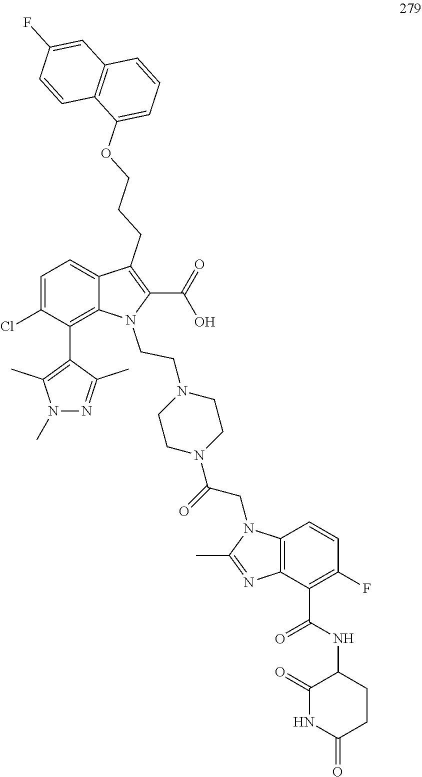 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)c(F)ccc54)CC3)c12]

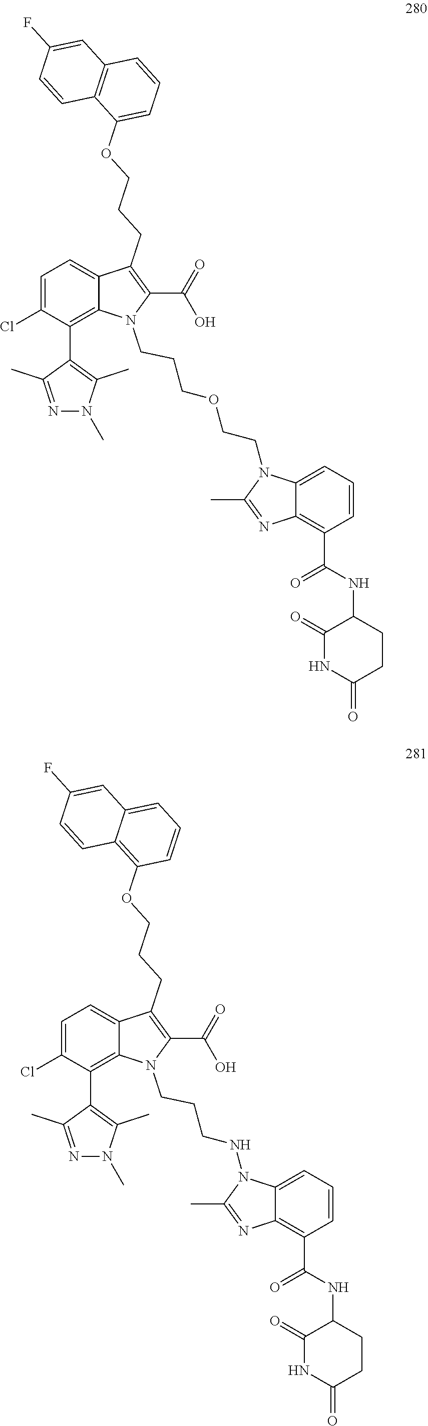 [SMILES: Cc1nn(C)c(C)c1-c1c(Cl)ccc2c(CCCOc3cccc4cc(F)ccc34)c(C(=O)O)n(CCCOCCn3c(C)nc4c(C(=O)NC5CCC(=O)NC5=O)cccc43)c12 ; Cc1nn(C)c(C)c1-c1c(Cl)ccc2c(CCCOc3cccc4cc(F)ccc34)c(C(=O)O)n(CCCNn3c(C)nc4c(C(=O)NC5CCC(=O)NC5=O)cccc43)c12]

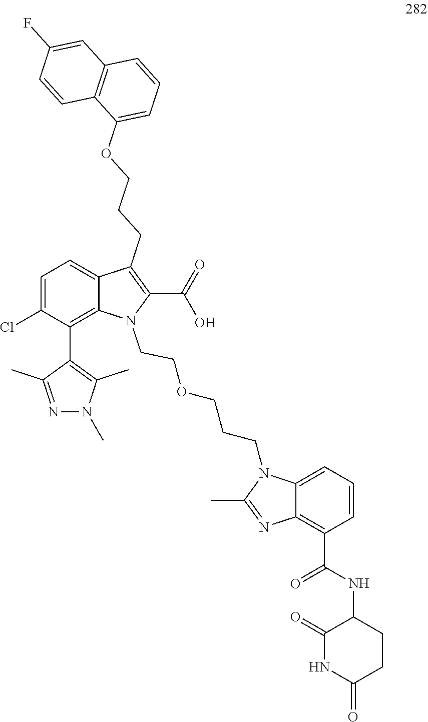 [SMILES: Cc1nn(C)c(C)c1-c1c(Cl)ccc2c(CCCOc3cccc4cc(F)ccc34)c(C(=O)O)n(CCOCCCn3c(C)nc4c(C(=O)NC5CCC(=O)NC5=O)cccc43)c12]

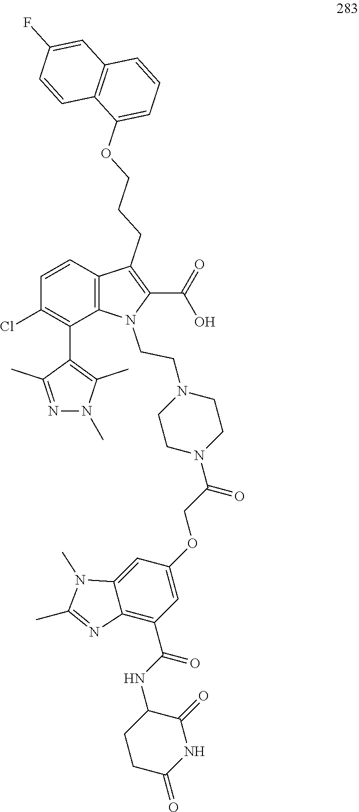 [SMILES: Cc1nn(C)c(C)c1-c1c(Cl)ccc2c(CCCOc3cccc4cc(F)ccc34)c(C(=O)O)n(CCN3CCN(C(=O)COc4cc(C(=O)NC5CCC(=O)NC5=O)c5nc(C)n(C)c5c4)CC3)c12]

In some embodiments, the compound is selected from:

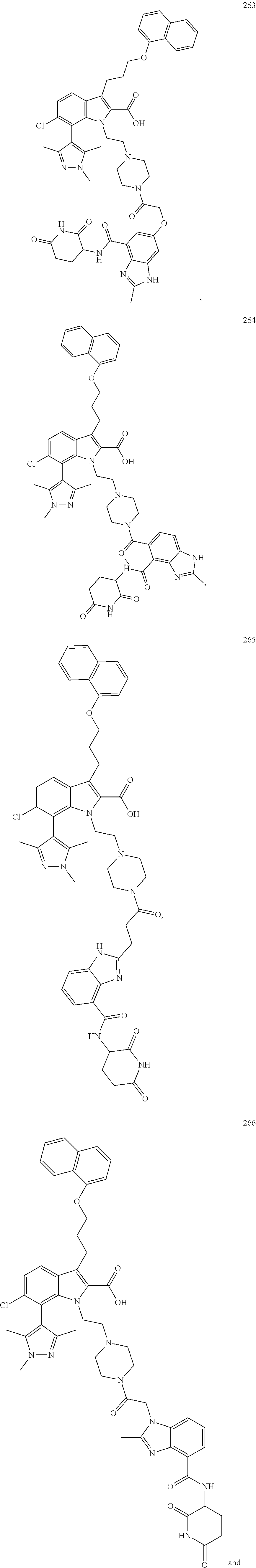

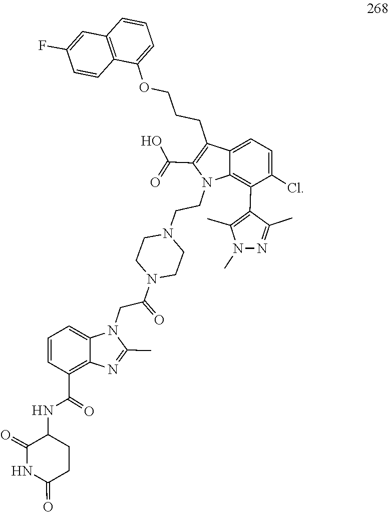 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cccc54)CC3)c12]

In some embodiments of any of the above aspects, T is C═O. In other embodiments, T is SO₂.

In some embodiments of any of the above aspects, X₁ and X₂ are O. In other embodiments, X₁ is O and X₂ is S. In other embodiments, X₁ is S and X₂ is O. In other embodiments, X₁ and X₂ are S.

In some embodiments of any of the above aspects, n is 0. In other embodiments, n is 1 or 2. In some embodiments, n is 1. In other embodiments, n is 2.

In some embodiments of any of the above aspects, [MCL-1 ligand moiety] is a compound of Formula (A), and R¹⁰ is —C₂₋₅alkyl-O—R¹³,

In some embodiments of any of the above aspects, R¹⁰ is —C₃H₆—O—R¹³.

In accordance with a fourth aspect of the invention, there is provided a pharmaceutical composition comprising a compound according to any of the above aspects of the present invention.

The invention also provides a compound or composition according to any of the above aspects of the present invention, for use in medicine.

The invention also provides a compound or composition according to any of the above aspects of the present invention, for use in the treatment of cancer. In some embodiments the cancer is selected from breast cancer, triple negative breast cancer, colorectal cancer, pancreatic cancer, skin cancer, melanoma, ovarian cancer, kidney cancer, lung cancer, small-cell lung cancer, non-small-cell lung cancer, lymphoma, non-Hodgkin's lymphoma, multiple myeloma, cervical cancer, leukaemia, chronic lymphocytic leukaemia (CLL), acute myeloid leukaemia (AML), chronic myelogenous leukaemia (CML), acute lymphoblastic leukaemia (ALL), bladder cancer, and prostate cancer. In some embodiments, the cancer is multiple myeloma or acute myeloid leukaemia.

The present invention also provides a method of treating cancer in a subject in need thereof, the method comprising administering to the subject an effective amount of a compound or composition according to any of the above aspects of the present invention. In some embodiments, the cancer is selected from breast cancer, triple negative breast cancer, colorectal cancer, pancreatic cancer, skin cancer, melanoma, ovarian cancer, kidney cancer, lung cancer, small-cell lung cancer, non-small-cell lung cancer, lymphoma, non-Hodgkin's lymphoma, multiple myeloma, cervical cancer, leukaemia, chronic lymphocytic leukaemia (CLL), acute myeloid leukaemia (AML), chronic myelogenous leukaemia (CML), acute lymphoblastic leukaemia (ALL), bladder cancer, and prostate cancer. In some embodiments, the cancer is multiple myeloma or acute myeloid leukaemia. In some embodiments, the administration does not result in cytotoxicity in cardiomyocytes in the subject. In some embodiments, the method further comprises administering at least one additional active agent to the subject. In some embodiments, the at least one additional active agent is an anti-cancer agent selected from eribulin; fulvestrant; midostaurin; an immune checkpoint inhibitor selected from anti-pd-1 antibody, anti-pd-11 antibody, and anti pd-1/pd-11 interaction inhibitor; nivolumab; pembrolizumab; atezolizumab; pidilizumab; carfilzomib; venetoclax; cytarabine; anthracyclines; a taxane compound; and hypomethylating agents.

The invention also provides a compound or composition according to any of the above aspects of the present invention, for use in reversing resistance to chemotherapy or targeted cancer therapies.

The invention also provides a method of reversing resistance to chemotherapy or targeted cancer therapies in a subject in need thereof, the method comprising administering to the subject an effective amount of a compound or composition of the present invention.

The invention also provides a combined preparation of a compound of the present invention and at least one additional active agent, for simultaneous, separate or sequential use in therapy. In some embodiments, the at least one additional active agent is an anti-cancer agent selected from eribulin; fulvestrant; midostaurin; an immune checkpoint inhibitor selected from anti-pd-1 antibody, anti-pd-11 antibody, and anti pd-1/pd-11 interaction inhibitor; nivolumab; pembrolizumab; atezolizumab; pidilizumab; carfilzomib; venetoclax; cytarabine; anthracyclines; a taxane compound; and hypomethylating agents. In some embodiments, the therapy is the treatment of cancer.

The invention also provides a compound of formula (X):

[MCL-1 inhibitor]-L-[cereblon binding moiety] (X)

wherein L is a bond or a linker compound.

In some embodiments, the cereblon binding moiety is a [ligase ligand moiety] of the present invention. In some embodiments, the MCL-1 inhibitor is an [MCL-1 ligand moiety] of the present invention. In some embodiments, the cereblon binding moiety is coupled to the MCL-1 inhibitor by a linker compound, wherein the linker compound is covalently attached to the cereblon binding moiety and the MCL-1 inhibitor. In some embodiments, the linker compound is a [linker] of the present invention.

The invention also provides a method of reducing the cardiac cytotoxicity of an MCL-1 inhibitor, comprising coupling a cereblon binding moiety to the MCL-1 inhibitor. In some embodiments, the cereblon binding moiety is a [ligase ligand moiety] of the present invention. In some embodiments, the MCL-1 inhibitor is an [MCL-1 ligand moiety] of the present invention. In some embodiments, the cereblon binding moiety is coupled to the MCL-1 inhibitor by a linker compound, wherein the linker compound is covalently attached to the cereblon binding moiety and the MCL-1 inhibitor. In some embodiments, the linker compound is a [linker] of the present invention.

As used herein the term “alkyl” is intended to include both unsubstituted alkyl groups, and alkyl groups which are substituted by one or more additional groups. In some embodiments, the alkyl group is an unsubstituted alkyl group. In some embodiments, the alkyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the alkyl group is a C₁-C₁₂ alkyl, a C₁-C₁₀ alkyl, a C₁-C₈ alkyl, a C₁-C₆ alkyl, or a C₁-C₄ alkyl group. In some embodiments the alkyl group is a linear alkyl group. In some embodiments the alkyl group is an unsubstituted linear alkyl group. In some embodiments the alkyl group is a linear alkyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments the alkyl group is a branched alkyl group. In some embodiments the alkyl group is an unsubstituted branched alkyl group. In some embodiments the alkyl group is a branched alkyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl.

As used herein the term “alkenyl” is intended to include both unsubstituted alkenyl groups, and alkenyl groups which are substituted by one or more additional groups. In some embodiments, the alkenyl group is an unsubstituted alkenyl group. In some embodiments, the alkenyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the alkenyl group is a C₂-C₁₂ alkenyl, a C₂-C₁₀ alkenyl, a C₂-C₈ alkenyl, a C₂-C₆ alkenyl, or a C₂-C₄ alkenyl group. In some embodiments the alkenyl group is a linear alkenyl group. In some embodiments the alkenyl group is an unsubstituted linear alkenyl group. In some embodiments the alkenyl group is a linear alkenyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments the alkenyl group is a branched alkenyl group. In some embodiments the alkenyl group is an unsubstituted branched alkenyl group. In some embodiments the alkenyl group is a branched alkenyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl.

As used herein the term “alkynyl” is intended to include both unsubstituted alkynyl groups, and alkynyl groups which are substituted by one or more additional groups. In some embodiments, the alkynyl group is an unsubstituted alkynyl group. In some embodiments, the alkynyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the alkynyl group is a C₂-C₁₂ alkynyl, a C₂-C₁₀ alkynyl, a C₂-C₈ alkynyl, a C₂-C₆ alkynyl, or a C₂-C₄ alkynyl group. In some embodiments the alkynyl group is a linear alkynyl group. In some embodiments the alkynyl group is an unsubstituted linear alkynyl group. In some embodiments the alkynyl group is a linear alkynyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments the alkynyl group is a branched alkynyl group. In some embodiments the alkynyl group is an unsubstituted branched alkynyl group. In some embodiments the alkynyl group is a branched alkynyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl.

As used herein the term “cycloalkyl” is intended to include both unsubstituted cycloalkyl groups, and cycloalkyl groups which are substituted by one or more additional groups. The term “cycloalkyl” is also intended to include monocyclic and bicyclic ring systems (including spirocyclic ring systems, in which the two rings share a single atom; fused bicyclic ring systems, in which the two rings share two adjacent atoms; and bridged bicyclic ring systems, in which the two rings share three or more atoms). In some embodiments, the cycloalkyl group is an unsubstituted cycloalkyl group. In some embodiments, the cycloalkyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the cycloalkyl group is a C₃-C₁₂ cycloalkyl, a C₄-C₁₂ cycloalkyl, a C₅-C₁₂ cycloalkyl, a C₃-C₁₀ cycloalkyl, a C₄-C₁₀ cycloalkyl, a C₅-C₁₀ cycloalkyl, a C₃-C₈ cycloalkyl, a C₄-C₈ cycloalkyl, a C₅-C₈ cycloalkyl, a C₃-C₆ cycloalkyl, a C₄-C₆ cycloalkyl, a C₅-C₆ cycloalkyl, a C₃-C₄ cycloalkyl, or a C₄-C₅ cycloalkyl group.

As used herein the term “cycloalkenyl” is intended to include both unsubstituted cycloalkenyl groups, and cycloalkenyl groups which are substituted by one or more additional groups. In some embodiments, the cycloalkenyl group is an unsubstituted cycloalkenyl group. In some embodiments, the cycloalkenyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the cycloalkenyl group is a C₄-C₁₂ cycloalkenyl, a C₅-C₁₂ cycloalkenyl, a C₄-C₁₀ cycloalkenyl, a C₅-C₁₀ cycloalkenyl, a C₄-C₈ cycloalkenyl, a C₅-C₈ cycloalkenyl, a C₄-C₆ cycloalkenyl, a C₅-C₆ cycloalkenyl, or a C₄-C₅ cycloalkenyl group.

As used herein the term “heterocycloalkyl” is intended to include both unsubstituted heterocycloalkyl groups, and heterocycloalkyl groups which are substituted by one or more additional groups. The term “heterocycloalkyl” is also intended to include monocyclic and bicyclic ring systems (including spirocyclic ring systems, in which the two rings share a single atom; fused bicyclic ring systems, in which the two rings share two adjacent atoms; and bridged bicyclic ring systems, in which the two rings share three or more atoms). In some embodiments, the heterocycloalkyl group is a monocyclic ring system, a spirocyclic ring system, or a fused bicyclic ring system. In some embodiments, the heterocycloalkyl group is an unsubstituted heterocycloalkyl group. In some embodiments, the heterocycloalkyl group is substituted by one or more groups selected from —R^{W}, —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, one or more —CH₂— groups of the heterocycloalkyl ring may be replaced with a —C(O)— group, In some embodiments, the heterocycloalkyl group is a C₃-C₁₂ heterocycloalkyl, a C₄-C₁₂ heterocycloalkyl, a C₅-C₁₂ heterocycloalkyl, a C₃-C₁₀ heterocycloalkyl, a C₄-C₁₀ heterocycloalkyl, a C₅-C₁₀ heterocycloalkyl, a C₃-C₈ heterocycloalkyl, a C₄-C₈ heterocycloalkyl, a C₅-C₈ heterocycloalkyl, a C₃-C₆ heterocycloalkyl, a C₄-C₆ heterocycloalkyl, a C₅-C₆ heterocycloalkyl, a C₃-C₄ heterocycloalkyl, or a C₄-C₅ heterocycloalkyl group.

As used herein the term “aryl” is intended to include both unsubstituted aryl groups, and aryl groups which are substituted by one or more additional groups. In some embodiments, the aryl group is an unsubstituted aryl group. In some embodiments, the aryl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the aryl group is a C₆-C₁₀ aryl, a C₆-C₈ aryl, or a C₆ aryl.

As used herein the term “heteroaryl” is intended to include both unsubstituted heteroaryl groups, and heteroaryl groups which are substituted by one or more additional groups. In some embodiments, the heteroaryl group is an unsubstituted heteroaryl group. In some embodiments, the heteroaryl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the heteroaryl group is a C₆-C₁₀ heteroaryl, a C₆-C₉ heteroaryl, a C₆-C₈ heteroaryl, or a C₆ heteroaryl.

As used herein the term “fused heterocycloalkyl-heteroaryl” is intended to mean a bicyclic ring system in which one ring is a heterocycloalkyl ring and the other is a heteroaryl ring, and in which the two rings share two adjacent atoms. Of the two adjacent atoms shared by the two rings, both may be carbon atoms; both may be heteroatoms (e. g. independently O, N or S); or one may be a carbon atom and the other a heteroatom (e. g. O, N or S). The fused heterocycloalkyl-heteroaryl may be unsubstituted or may be substituted by one or more additional groups. In some embodiments, the fused heterocycloalkyl-heteroaryl group is an unsubstituted cycloalkenyl group. In some embodiments, the fused heterocycloalkyl-heteroaryl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl.

As used herein the term “benzyl” is intended to include both unsubstituted benzyl groups, and benzyl groups which are substituted by one or more additional groups. In some embodiments, the benzyl group is an unsubstituted benzyl group. In some embodiments, the benzyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl.

In some embodiments of any of the above aspects of the invention, all alkyl, alkenyl, alkynyl, cycloalkyl, cycloalkenyl, heterocycloalkyl, aryl, heteroaryl and benzyl groups in the compounds are unsubstituted.

### BRIEF DESCRIPTION OF THE FIGURES

FIG. 1 is a schematic illustration of the general principle for targeted protein degradation upon treatment with a bifunctional compound. Bifunctional compounds of the present invention comprise an E3 ligase binding moiety (LBM) on the one end and an MCL-1 ligand moiety on the other end (MLM).

FIG. 2 is an assay showing the dose-dependent effect of various compounds of the invention on the level of MCL-1 protein in the OPM-2 cell line after 6 h treatment (2A) and 24 h treatment (2B)

FIG. 3 is an assay showing the dose-dependent effect of compounds of the invention and reference compounds on the level of MCL-1 protein in the OPM-2 (FIG. 3A, 3C, 3D), MV-4-11 (FIG. 3B) DMS 114 (FIG. 3E) cell lines after 3 h, 6 h, and/or 24 h treatment, as indicated.

FIG. 4 is an assay showing the dose-dependent effect of compound 204 of the invention on the viability of OPM-2, MV-4-11 and ARH-77 cells

FIG. 5 is an assay showing the effect of compound of the invention and the reference compound on caspase 3/7 activity in iPSC-derived cardiomyocytes (iPSC-CMs) (NB 100 μM Concentration point for AZD-5991 is not displayed because of the excessive cell death)

FIG. 6 is an assay showing the dose-dependent effect of compounds of the invention on the population of late apoptotic/cell death (Annexin+/PI+) cells after 24 hours of treatment.

FIG. 7 presents the effect of a compound of the invention on the mitochondrial potential of MV-4-11 cells.

FIG. 8 shows the concentration-dependent decrease (compared to DMSO control) in the population of MV-4-11 cells with normal polarized mitochondria after treatment of the cells with the compounds of the present invention.

### DETAILED DESCRIPTION OF THE INVENTION

As discussed above, the present invention provides a compound of formula (I)

[MCL-1ligand moiety]-linker-[ligase ligand moiety] (I)

or a salt, solvate, hydrate, isomer or prodrug thereof,
wherein [ligase ligand moiety] is:

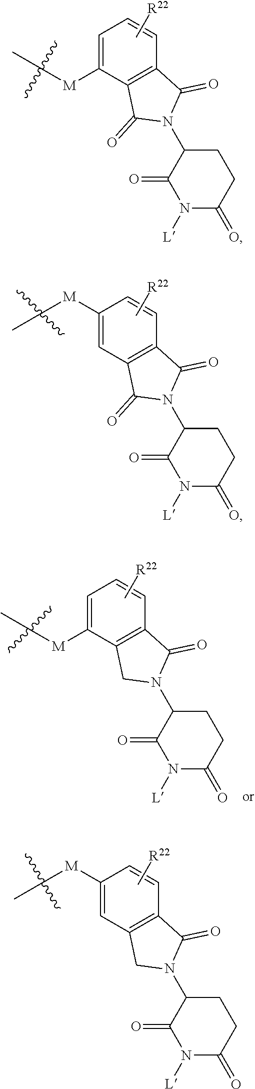

or
Formula (IV)

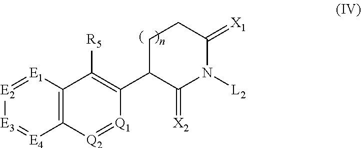

or
Formula (Va) or (Vb):

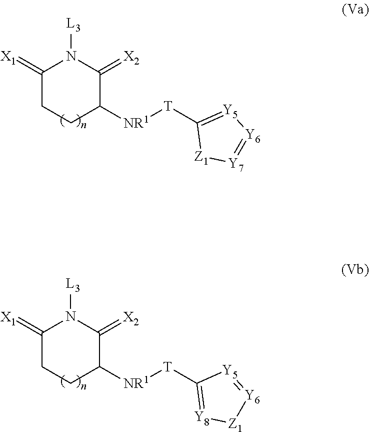

or
Formula (IIa) or (IIb):

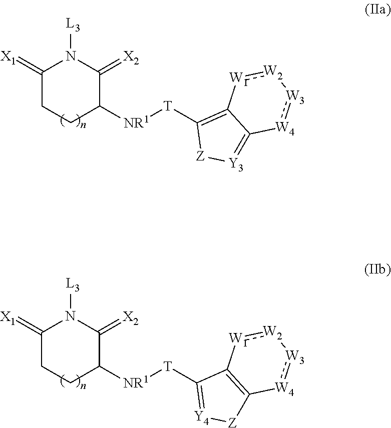

or
Formula (II):

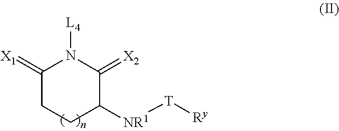

or
Formula (III):

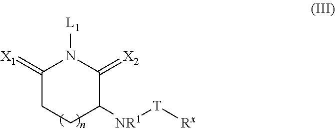

wherein [MCL-1 ligand moiety] is a compound of Formula (A), Formula (B) or Formula (C)

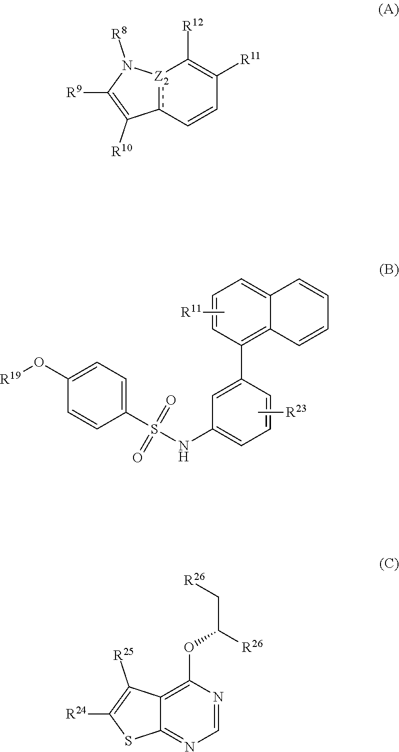

and wherein [linker] has the following formula

R¹⁴—R¹⁵—R¹⁶—R¹⁷—R¹⁸

wherein
R¹⁴ is —C₁₋₆alkyl, —C₂₋₆alkenyl, —C₂₋₆alkynyl, C₁₋₆ alkyl-N(C₁₋₆ alkyl)-, —C(O)—, —SO₂— or is absent
R¹⁵ is cycloalkyl, heterocycloalkyl, aryl, heteroaryl, —C₁₋₆ alkyl-NH—, C₁₋₆ alkyl-N(C₁₋₆ alkyl)-, -cycloalkyl-NH—, heterocycloalkyl-NH—, or is absent
R¹⁶ is —C₁₋₆alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, (C₂H₄—O)ₓ, (C₃H₆—O)ₓ, or is absent
x is 1-10
y is 2-10
R¹⁸ is —C₁₋₆ alkyl, heterocycloalkyl, or is absent
   wherein at least one of R¹⁴-R¹⁸ is present

### Ligase Ligand Moieties

Ligase Ligand Moieties with Thalidomide-Type Structure

In one aspect, the ligase ligand moiety is:

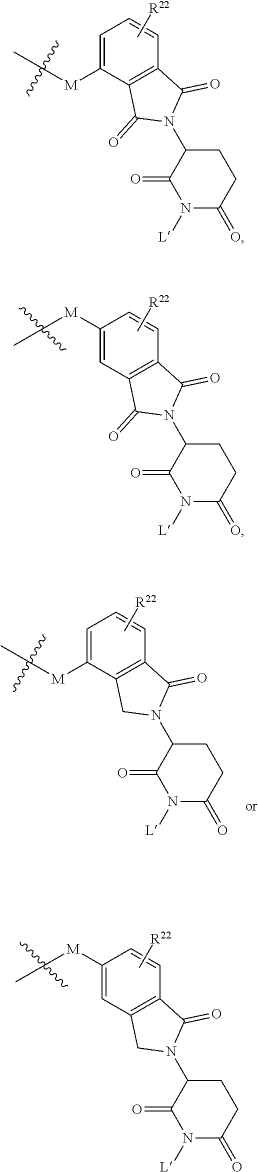

wherein
M is O, S, or NH, or is absent;
[symbol] indicates attachment to R¹⁸ of the linker;
R²² is hydrogen, halogen or an amino group; and
L′ is hydrogen, alkyl, benzyl, acetyl or pivaloyl.

In some embodiments, M is O or NH, or is absent.

Examples of the above ligase ligand moieties are shown in Table 1 below:

TABLE 1

| Compound number | Structure |
|---|---|
| 108 | 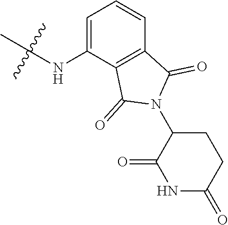 [SMILES: *Nc1cccc2c1C(=O)N(C1CCC(=O)NC1=O)C2=O] |
| 109 | 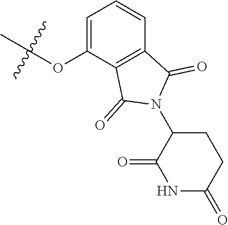 [SMILES: *Oc1cccc2c1C(=O)N(C1CCC(=O)NC1=O)C2=O] |
| 110 | 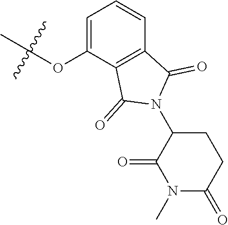 [SMILES: *Oc1cccc2c1C(=O)N(C1CCC(=O)N(C)C1=O)C2=O] |
| 111 | 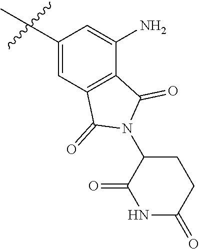 [SMILES: *c1cc(N)c2c(c1)C(=O)N(C1CCC(=O)NC1=O)C2=O] |
| 112 | 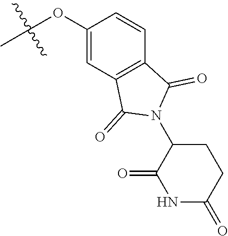 [SMILES: *Oc1ccc2c(c1)C(=O)N(C1CCC(=O)NC1=O)C2=O] |
| 113 | 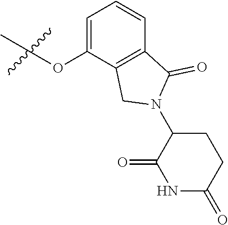 [SMILES: *Oc1cccc2c1CN(C1CCC(=O)NC1=O)C2=O] |
| 114 | 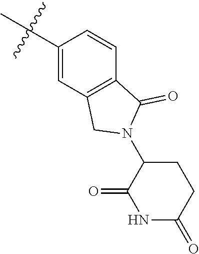 [SMILES: *c1ccc2c(c1)CN(C1CCC(=O)NC1=O)C2=O] |
| 115 | 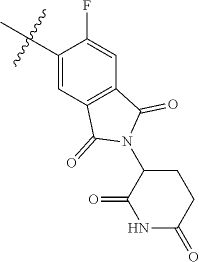 [SMILES: *c1cc2c(cc1F)C(=O)N(C1CCC(=O)NC1=O)C2=O] |
| | 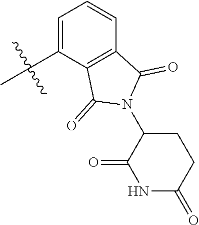 [SMILES: *c1cccc2c1C(=O)N(C1CCC(=O)NC1=O)C2=O] |
| | 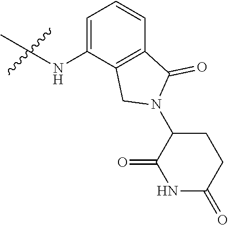 [SMILES: *Nc1cccc2c1CN(C1CCC(=O)NC1=O)C2=O] |
| | 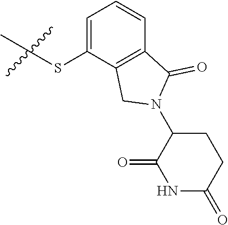 [SMILES: *Sc1cccc2c1CN(C1CCC(=O)NC1=O)C2=O] |
| | 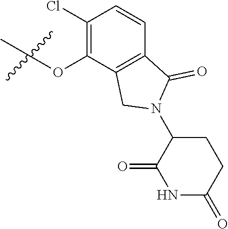 [SMILES: *Oc1c(Cl)ccc2c1CN(C1CCC(=O)NC1=O)C2=O] |
| | 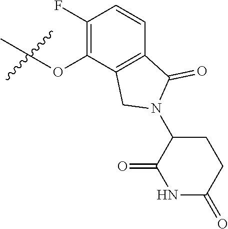 [SMILES: *Oc1c(F)ccc2c1CN(C1CCC(=O)NC1=O)C2=O] |
| | 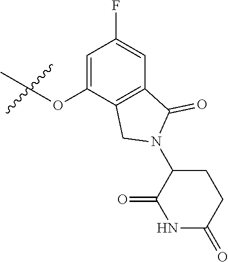 [SMILES: *Oc1cc(F)cc2c1CN(C1CCC(=O)NC1=O)C2=O] |
| | 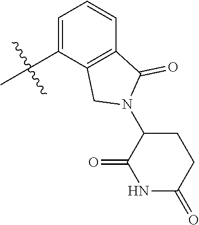 [SMILES: *c1cccc2c1CN(C1CCC(=O)NC1=O)C2=O] |
| | 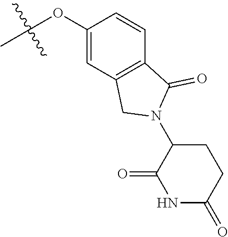 [SMILES: *Oc1ccc2c(c1)CN(C1CCC(=O)NC1=O)C2=O] |
| | 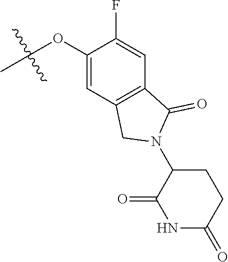 [SMILES: *Oc1cc2c(cc1F)C(=O)N(C1CCC(=O)NC1=O)C2] |

Compounds 108-110 and 112-115 are commercially available in the forms shown below. The synthesis of compound 111 is disclosed in the Examples section, below.

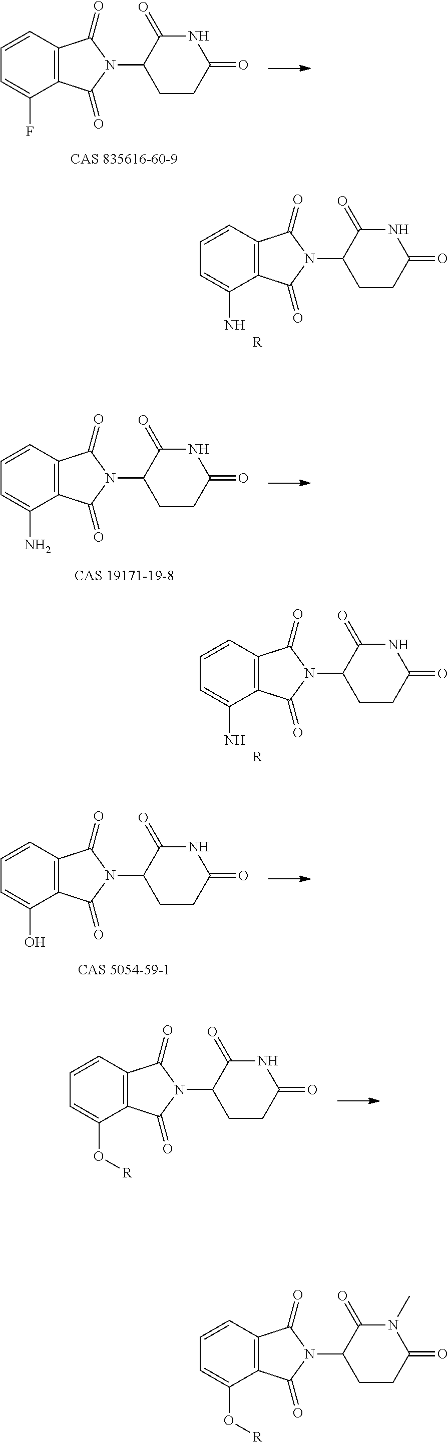

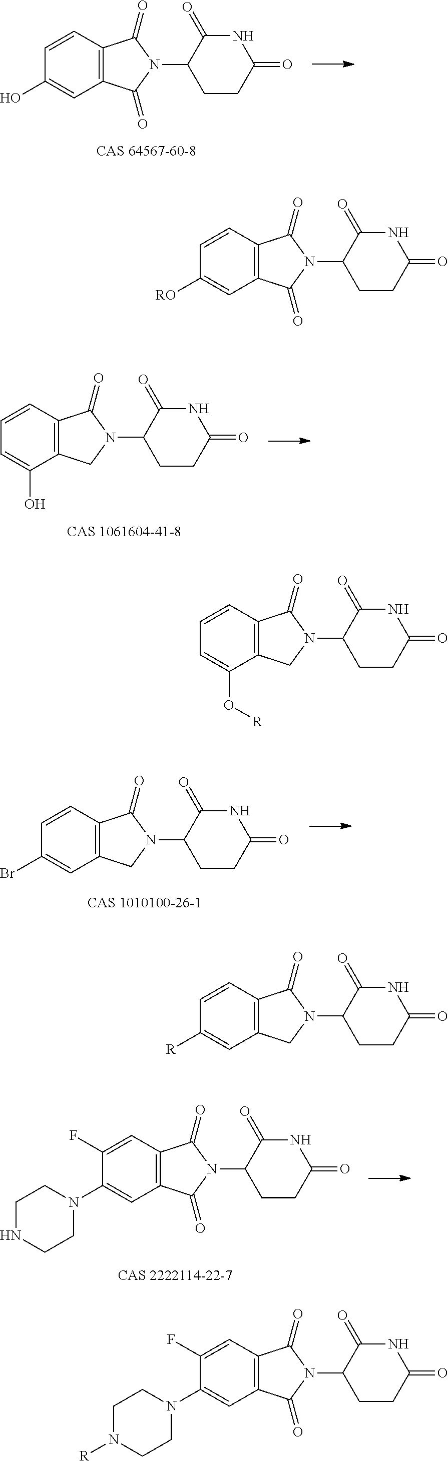

In some embodiments of the above ligase ligands, the

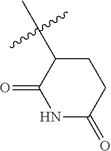 [SMILES: *C1CCC(=O)NC1=O]

moiety may be replaced with one of the following moieties:
wherein

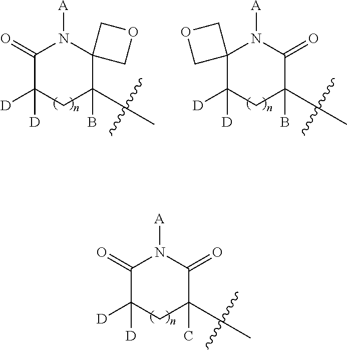

A is, hydrogen, alkyl, alkenyl, benzyl, aryl, heteroaryl, haloalkyl, haloalkenyl, —CH₂OC(O)^{t}Bu, —CH₂C(O)OR²⁷, —C(O)R²⁷, —C(O)OR²⁷, —C(O)NH₂, —C(O)NHR²⁷, —C(O)NR²⁷², —OR²⁷, —NR²⁷², —S(O)₂R²⁷ or P(O)(OR²⁷)(OR²⁷), wherein each R²⁷ is independently hydrogen, alkyl, cycloalkyl, alkenyl, cycloalkenyl, aryl, heteroaryl, or benzyl,
B is hydrogen, deuterium or alkyl,
C is hydrogen, deuterium or alkyl.

### Ligase Ligand Moieties of Formula (II) and Formula (III)

The synthesis of the ligase ligand moieties of Formula (II) and Formula (III) (as defined above) can be summarized as follows:

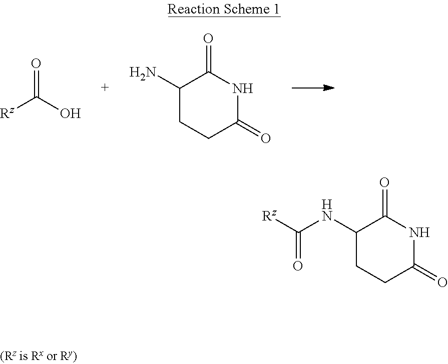

Example ligase ligand moieties of Formula (II) and Formula (III) are shown in Table 2 below. Compounds 4-6, 29, 39-41, 50-54, 58 and 62 could be modified to allow attachment to the [linker] (for example, by bromination of the aromatic ring followed by attachment—by palladium coupling—of either the [linker] itself, or of a functional group to which the [linker] could be attached).

TABLE 2

| Compound number | Structure |
|---|---|
| 1 | 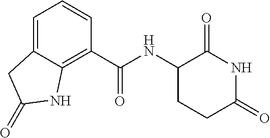 [SMILES: O=C1CCC(NC(=O)c2cccc3c2NC(=O)C3)C(=O)N1] |
| 2 | 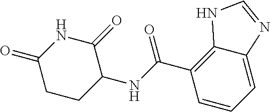 [SMILES: O=C1CCC(NC(=O)c2cccc3nc[nH]c23)C(=O)N1] |
| 3 | 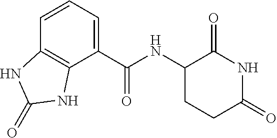 [SMILES: O=C1CCC(NC(=O)c2cccc3[nH]c(=O)[nH]c23)C(=O)N1] |
| 4 | 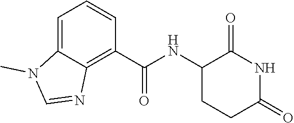 [SMILES: Cn1cnc2c(C(=O)NC3CCC(=O)NC3=O)cccc21] |
| 5 | 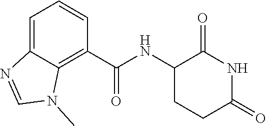 [SMILES: Cn1cnc2cccc(C(=O)NC3CCC(=O)NC3=O)c21] |
| 6 | 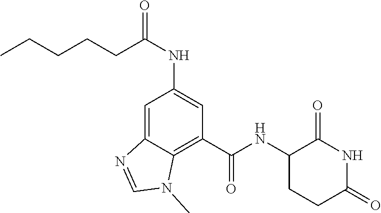 [SMILES: CCCCCC(=O)Nc1cc(C(=O)NC2CCC(=O)NC2=O)c2c(c1)ncn2C] |
| 7 | 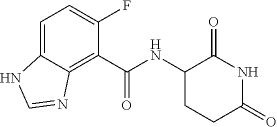 [SMILES: O=C1CCC(NC(=O)c2c(F)ccc3[nH]cnc23)C(=O)N1] |
| 8 | 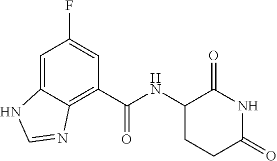 [SMILES: O=C1CCC(NC(=O)c2cc(F)cc3[nH]cnc23)C(=O)N1] |
| 9 | 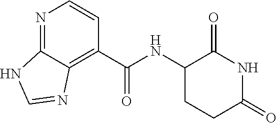 [SMILES: O=C1CCC(NC(=O)c2ccnc3[nH]cnc23)C(=O)N1] |
| 10 | 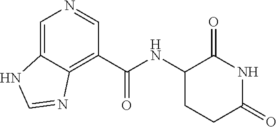 [SMILES: O=C1CCC(NC(=O)c2cncc3[nH]cnc23)C(=O)N1] |
| 11 | 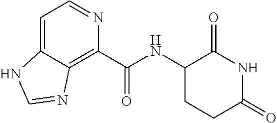 [SMILES: O=C1CCC(NC(=O)c2nccc3[nH]cnc23)C(=O)N1] |
| 12 | 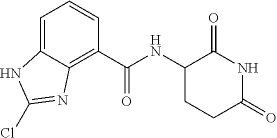 [SMILES: O=C1CCC(NC(=O)c2cccc3[nH]c(Cl)nc23)C(=O)N1] |
| 14 | 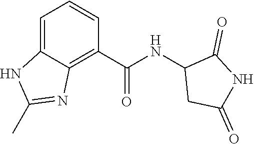 [SMILES: Cc1nc2c(C(=O)NC3CC(=O)NC3=O)cccc2[nH]1] |
| 15 | 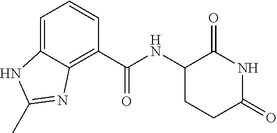 [SMILES: Cc1nc2c(C(=O)NC3CCC(=O)NC3=O)cccc2[nH]1] |
| 17 | 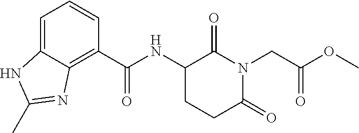 |
| 19 | 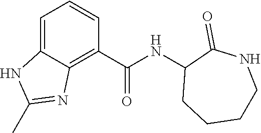 [SMILES: Cc1nc2c(C(=O)NC3CCCCNC3=O)cccc2[nH]1] |
| 20 | 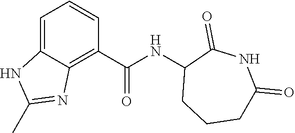 [SMILES: Cc1nc2c(C(=O)NC3CCCC(=O)NC3=O)cccc2[nH]1] |
| 22 | 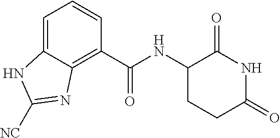 [SMILES: N#Cc1nc2c(C(=O)NC3CCC(=O)NC3=O)cccc2[nH]1] |
| 23 | 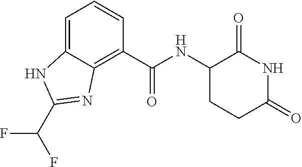 [SMILES: O=C1CCC(NC(=O)c2cccc3[nH]c(C(F)F)nc23)C(=O)N1] |
| 24 | 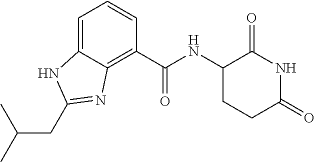 [SMILES: CC(C)Cc1nc2c(C(=O)NC3CCC(=O)NC3=O)cccc2[nH]1] |
| 25 | 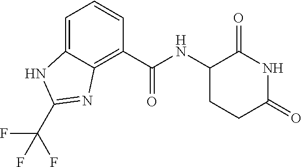 [SMILES: O=C1CCC(NC(=O)c2cccc3[nH]c(C(F)(F)F)nc23)C(=O)N1] |
| 26 | 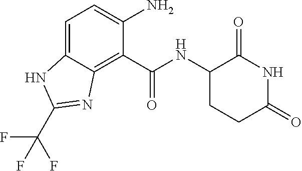 [SMILES: Nc1ccc2[nH]c(C(F)(F)F)nc2c1C(=O)NC1CCC(=O)NC1=O] |
| 27 | 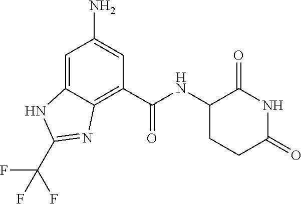 [SMILES: Nc1cc(C(=O)NC2CCC(=O)NC2=O)c2nc(C(F)(F)F)[nH]c2c1] |
| 28 | 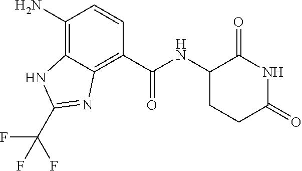 [SMILES: Nc1ccc(C(=O)NC2CCC(=O)NC2=O)c2nc(C(F)(F)F)[nH]c12] |
| 29 | 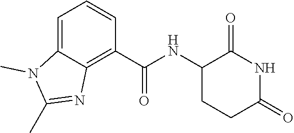 [SMILES: Cc1nc2c(C(=O)NC3CCC(=O)NC3=O)cccc2n1C] |
| 30 | 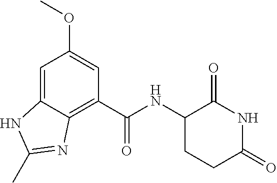 [SMILES: COc1cc(C(=O)NC2CCC(=O)NC2=O)c2nc(C)[nH]c2c1] |
| 31 | 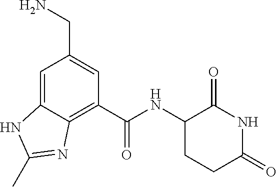 [SMILES: Cc1nc2c(C(=O)NC3CCC(=O)NC3=O)cc(CN)cc2[nH]1] |
| 32 | 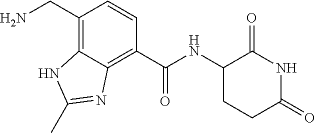 [SMILES: Cc1nc2c(C(=O)NC3CCC(=O)NC3=O)ccc(CN)c2[nH]1] |
| 33 | 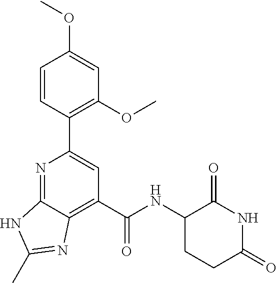 [SMILES: COc1ccc(-c2cc(C(=O)NC3CCC(=O)NC3=O)c3nc(C)[nH]c3n2)c(OC)c1] |
| 35 | 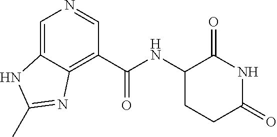 [SMILES: Cc1nc2c(C(=O)NC3CCC(=O)NC3=O)cncc2[nH]1] |
| 36 | 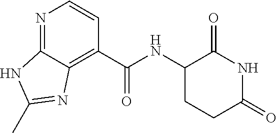 [SMILES: Cc1nc2c(C(=O)NC3CCC(=O)NC3=O)ccnc2[nH]1] |
| 37 | 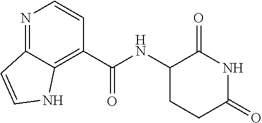 [SMILES: O=C1CCC(NC(=O)c2ccnc3cc[nH]c23)C(=O)N1] |
| 38 | 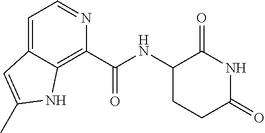 [SMILES: Cc1cc2ccnc(C(=O)NC3CCC(=O)NC3=O)c2[nH]1] |
| 39 | 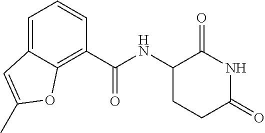 [SMILES: Cc1cc2cccc(C(=O)NC3CCC(=O)NC3=O)c2o1] |
| 40 | 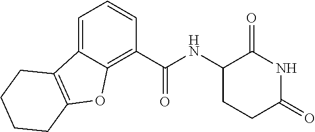 [SMILES: O=C1CCC(NC(=O)c2cccc3c4c(oc23)CCCC4)C(=O)N1] |
| 41 | 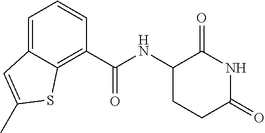 [SMILES: Cc1cc2cccc(C(=O)NC3CCC(=O)NC3=O)c2s1] |
| 42 | 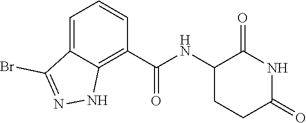 [SMILES: O=C1CCC(NC(=O)c2cccc3c(Br)n[nH]c23)C(=O)N1] |
| 43 | 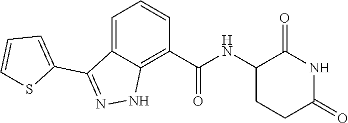 [SMILES: O=C1CCC(NC(=O)c2cccc3c(-c4cccs4)n[nH]c23)C(=O)N1] |
| 45 | 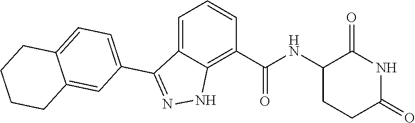 [SMILES: O=C1CCC(NC(=O)c2cccc3c(-c4ccc5c(c4)CCCC5)n[nH]c23)C(=O)N1] |
| 46 | 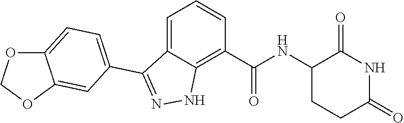 [SMILES: O=C1CCC(NC(=O)c2cccc3c(-c4ccc5c(c4)OCO5)n[nH]c23)C(=O)N1] |
| 47 | 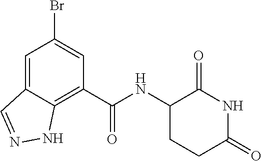 [SMILES: O=C1CCC(NC(=O)c2cc(Br)cc3cn[nH]c23)C(=O)N1] |
| 48 | 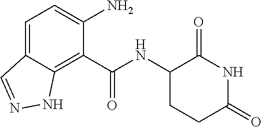 [SMILES: Nc1ccc2cn[nH]c2c1C(=O)NC1CCC(=O)NC1=O] |
| 50 | 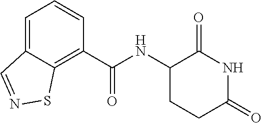 [SMILES: O=C1CCC(NC(=O)c2cccc3cnsc23)C(=O)N1] |
| 51 | 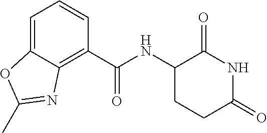 [SMILES: Cc1nc2c(C(=O)NC3CCC(=O)NC3=O)cccc2o1] |
| 52 | 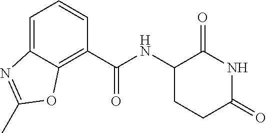 [SMILES: Cc1nc2cccc(C(=O)NC3CCC(=O)NC3=O)c2o1] |
| 53 | 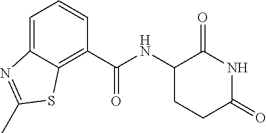 [SMILES: Cc1nc2cccc(C(=O)NC3CCC(=O)NC3=O)c2s1] |
| 54 | 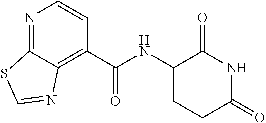 [SMILES: O=C1CCC(NC(=O)c2ccnc3scnc23)C(=O)N1] |
| 55 | 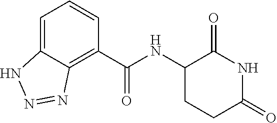 [SMILES: O=C1CCC(NC(=O)c2cccc3[nH]nnc23)C(=O)N1] |
| 57 | 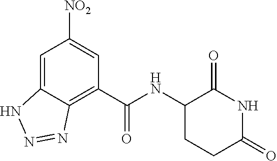 [SMILES: O=C1CCC(NC(=O)c2cc([N+](=O)[O-])cc3[nH]nnc23)C(=O)N1] |
| 58 | 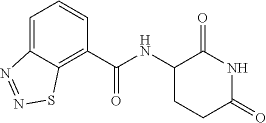 [SMILES: O=C1CCC(NC(=O)c2cccc3nnsc23)C(=O)N1] |
| 59 | 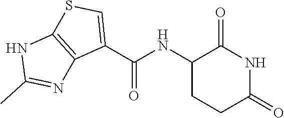 [SMILES: Cc1nc2c(C(=O)NC3CCC(=O)NC3=O)csc2[nH]1] |
| 60 | 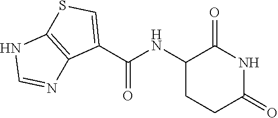 [SMILES: O=C1CCC(NC(=O)c2csc3[nH]cnc23)C(=O)N1] |
| 61 | 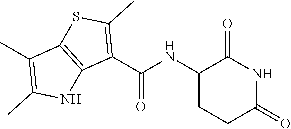 [SMILES: Cc1[nH]c2c(C(=O)NC3CCC(=O)NC3=O)c(C)sc2c1C] |
| 62 | 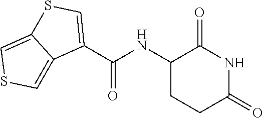 [SMILES: O=C1CCC(NC(=O)c2csc3cscc23)C(=O)N1] |

In some embodiments of Formula (II) and Formula (III), the

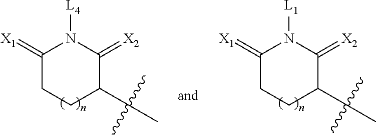

moieties may be replaced with one of the following moieties:

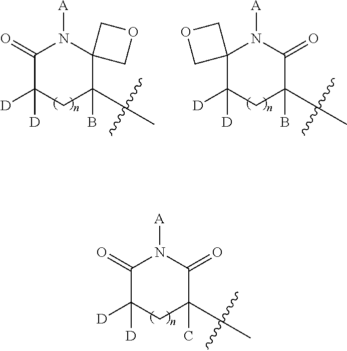

wherein
A is, hydrogen, alkyl, alkenyl, benzyl, aryl, heteroaryl, haloalkyl, haloalkenyl, —CH₂OC(O)^{t}Bu, —CH₂C(O)OR²⁷, —C(O)R²⁷, —C(O)OR²⁷, —C(O)NH₂, —C(O)NHR²⁷, —C(O)NR²⁷², —OR²⁷, —NR²⁷², —S(O)₂R²⁷ or P(O)(OR²⁷)(OR²⁷), wherein each R²⁷ is independently hydrogen, alkyl, cycloalkyl, alkenyl, cycloalkenyl, aryl, heteroaryl, or benzyl,
B is hydrogen, deuterium or alkyl,
C is hydrogen, deuterium or alkyl,
each D is independently deuterium or hydrogen,
   as shown, for example, in the compounds below:

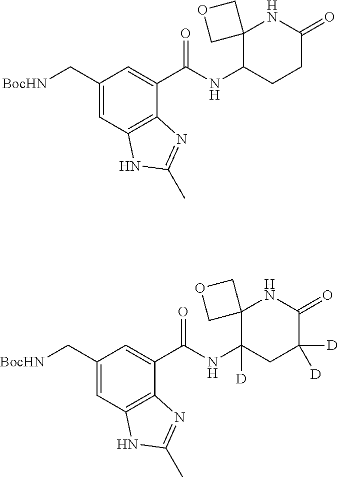 [SMILES: Cc1nc2c(C(=O)NC3CCC(=O)NC34COC4)cc(CNC(=O)OC(C)(C)C)cc2[nH]1 ; [2H]C1([2H])CC([2H])(NC(=O)c2cc(CNC(=O)OC(C)(C)C)cc3[nH]c(C)nc23)C2(COC2)NC1=O]

Ligase ligand moieties of Formula (IV)

The synthesis of the ligase ligand moieties of Formula (IV) (as defined above) can be summarized as follows:

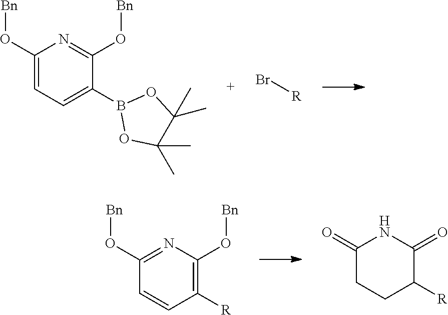

Example ligase ligand moieties of Formula (IV) are shown in Table 3 below. Compound 65 could be modified to allow attachment to the [linker] (e.g. by nucleophilic aromatic substitution; or by exchange of fluorine for bromine followed by attachment—by palladium coupling—of either the [linker] itself, or of a functional group to which the [linker] could be attached).

TABLE 3

| Compound number | Structure |
|---|---|
| 63 | 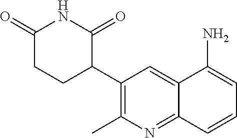 [SMILES: Cc1nc2cccc(N)c2cc1C1CCC(=O)NC1=O] |
| 64 | 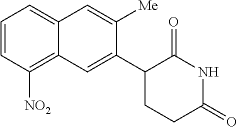 [SMILES: Cc1cc2cccc([N+](=O)[O-])c2cc1C1CCC(=O)NC1=O] |
| 65 | 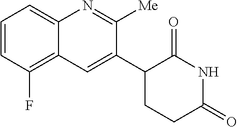 [SMILES: Cc1nc2cccc(F)c2cc1C1CCC(=O)NC1=O] |

### Ligase Ligand Moieties of Formula (Va) and Formula (Vb), and Formula (IIa) and Formula (IIb)

The synthesis of the ligase ligand moieties of Formulae (Va), (Vb), (IIa) and (IIb) (as defined above) can be summarized in the following general procedure (carried out under Synthetic Conditions D, E, F or G, as set out below:

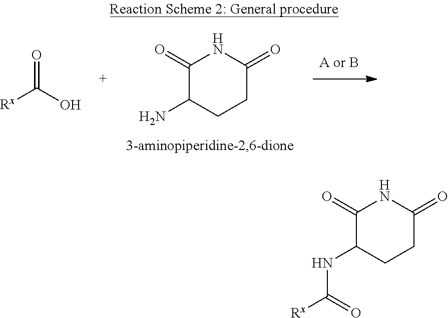

### Synthetic Conditions D

An appropriate acid (R^{x}COOH in the above reaction scheme) (1.1 eq), DMAP (0.04 eq), and EDC (1.2 eq) were added to a solution of 3-aminopiperidine-2,6-dione (1 eq) and N-hydroxybenzotriazole (1.2 eq) in DMF (0.5 M). The reaction mixture was stirred overnight at room temperature (20-25° C.). Water (2×DMF volume) was added and the obtained solution was extracted with dichloromethane (3×DMF volume). The combined organic layers were washed with water, dried over Na₂SO₄, and concentrated under reduced pressure. The crude product was purified by preparative HPLC or by column chromatography.

### Synthetic Conditions E

An appropriate acid R^{x}COOH in the above reaction scheme) (1 eq) and EDC (1.2 eq) were added to a solution of 3-aminopiperidine-2,6-dione (hydrochloride salt, 1.1 eq), triethylamine (1.2 eq) and N-hydroxybenzotriazole (1.2 eq) in DMA (0.5 M). The reaction mixture was stirred overnight at rt. Water (2×DMA volume) was added and obtained mixture was extracted with dichloromethane (3×DMA volume). The combined organic layers were washed with water, dried over Na₂SO₄, and concentrated under reduced pressure. The crude product was isolated by preparative HPLC or by column chromatography.

### Synthetic Conditions F

To a solution of appropriate acid (R^{x}COOH in the above reaction scheme) (1 eq) and HATU (1.5 eq) in dry DMF were added 3-aminopiperidine-2,6-dione (hydrochloride salt, 1.2 eq) and DIPEA (3 eq). The reaction mixture was stirred overnight at rt. The crude product was purified by preparative HPLC or/and by preparative TLC.

### Synthetic Conditions G

To a solution of appropriate acid (R^{x}COOH in the above reaction scheme) (1 eq) 3-aminopiperidine-2,6-dione (hydrochloride salt, 1.2 eq) and DMAP (0.1 eq.) in an inert atmosphere in dry DMF were added DIPEA (2.2 eq.) and HATU (1.5 eq) in dry DMF. The reaction mixture was stirred overnight at rt. The crude product was purified by preparative HPLC or/and by preparative TLC.

### Example Method 1: Formation of Chlorinated R^{x} Group of R^{x}COOH (or its Ester R^{x}COOR^{y})

NCS (1.1 eq) was added to a solution of an appropriate starting material (1 eq) in DMF (0.5 M) and the reaction mixture was stirred for 2 h at room temperature (20-25° C.). The reaction mixture was poured into water (2×DMF volume) and occurred precipitate was filtered. The solids were washed with water and dried in vacuum to give the acid, ROOH.

### Example Method 2: Synthesis of R^{x}COOH from Corresponding Ester R^{x}COOR^{y})

LiOH (1.1 eq) was added to a solution of an appropriate ester (1 eq) in THF:water mixture (3:1 or 5:1, 85 mM) and the resulting mixture was stirred overnight at room temperature (20-25° C.). The mixture was concentrated under reduced pressure, diluted with water, and acidified with concentrated HCl to pH=2-3. The precipitate was filtered, washed with water, and dried in vacuum to give the target carboxylic acid.

### Example Method 3: Formation of Acetylated R^{x} Group of R^{x}COOR^{y}

A mixture of an appropriate amine (1 eq.), Ac₂O (3 eq.), and DMAP (0.2 eq.) in dioxane (0.2 M) was heated to 80° C. for 2 h. Upon completion, the mixture was cooled down to room temperature (20-25° C.) and concentrated under reduced pressure. The residue was diluted with water (1× dioxane volume) and extracted with EtOAc (3× dioxane volume). The organic layers were washed with water, brine, dried over Na₂SO₄, and evaporated to dryness to afford an acylated product typically used without further purification.

Example ligase ligand moieties of Formula (Va) and Formula (Vb) are shown in Table 4 below. Compounds 66-74, 77-86, 88, 90-92, 96, 97 and 100 could be modified to allow attachment to the [linker] (e.g. by C—H bond activation).

TABLE 4

| Compound number | Structure |
|---|---|
| 66 | 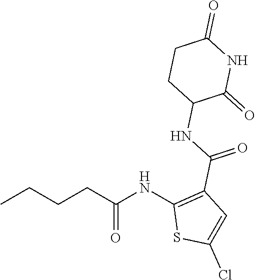 [SMILES: CCCCC(=O)Nc1sc(Cl)cc1C(=O)NC1CCC(=O)NC1=O] |
| 67 | 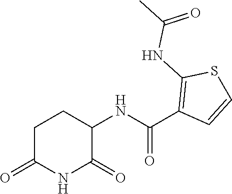 [SMILES: CC(=O)Nc1sccc1C(=O)NC1CCC(=O)NC1=O] |
| 68 | 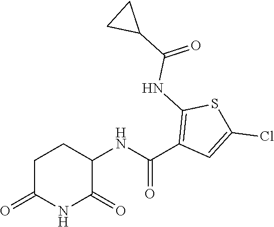 [SMILES: O=C1CCC(NC(=O)c2cc(Cl)sc2NC(=O)C2CC2)C(=O)N1] |
| 69 | 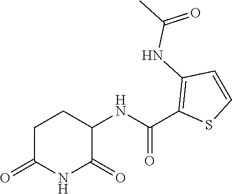 [SMILES: CC(=O)Nc1ccsc1C(=O)NC1CCC(=O)NC1=O] |
| 70 | 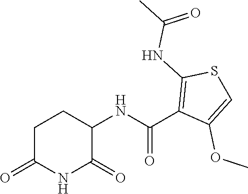 [SMILES: COc1csc(NC(C)=O)c1C(=O)NC1CCC(=O)NC1=O] |
| 71 | 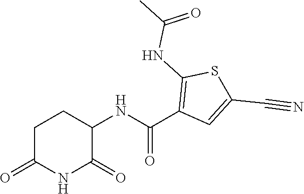 [SMILES: CC(=O)Nc1sc(C#N)cc1C(=O)NC1CCC(=O)NC1=O] |
| 72 | 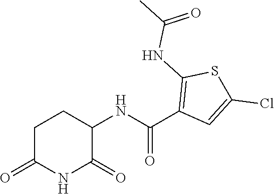 [SMILES: CC(=O)Nc1sc(Cl)cc1C(=O)NC1CCC(=O)NC1=O] |
| 73 | 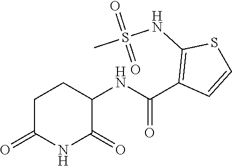 [SMILES: CS(=O)(=O)Nc1sccc1C(=O)NC1CCC(=O)NC1=O] |
| 74 | 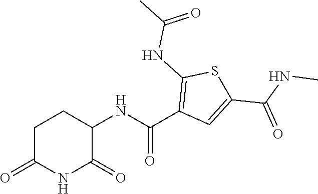 [SMILES: CNC(=O)c1cc(C(=O)NC2CCC(=O)NC2=O)c(NC(C)=O)s1] |
| 75 | 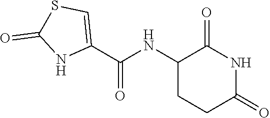 [SMILES: O=C1CCC(NC(=O)c2csc(=O)[nH]2)C(=O)N1] |
| 76 | 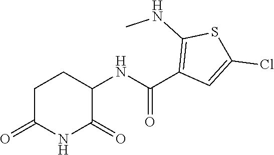 [SMILES: CNc1sc(Cl)cc1C(=O)NC1CCC(=O)NC1=O] |
| 77 | 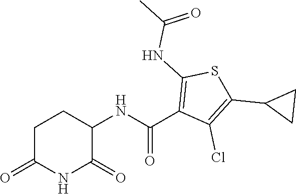 [SMILES: CC(=O)Nc1sc(C2CC2)c(Cl)c1C(=O)NC1CCC(=O)NC1=O] |
| 78 | 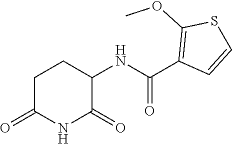 [SMILES: COc1sccc1C(=O)NC1CCC(=O)NC1=O] |
| 79 | 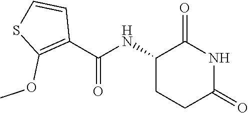 [SMILES: COc1sccc1C(=O)N[C@H]1CCC(=O)NC1=O] |
| 80 | 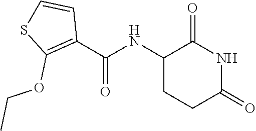 [SMILES: CCOc1sccc1C(=O)NC1CCC(=O)NC1=O] |
| 81 | 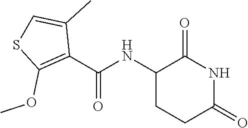 [SMILES: COc1scc(C)c1C(=O)NC1CCC(=O)NC1=O] |
| 82 | 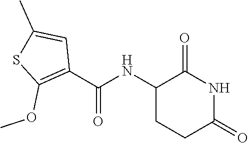 [SMILES: COc1sc(C)cc1C(=O)NC1CCC(=O)NC1=O] |
| 83 | 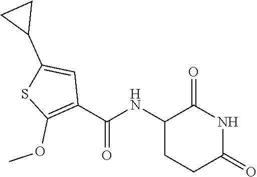 [SMILES: COc1sc(C2CC2)cc1C(=O)NC1CCC(=O)NC1=O] |
| 84 | 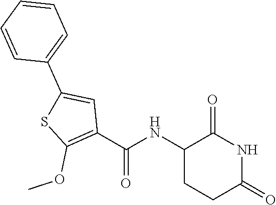 [SMILES: COc1sc(-c2ccccc2)cc1C(=O)NC1CCC(=O)NC1=O] |
| 85 | 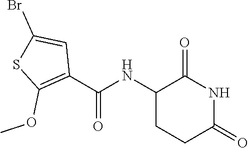 [SMILES: COc1sc(Br)cc1C(=O)NC1CCC(=O)NC1=O] |
| 86 | 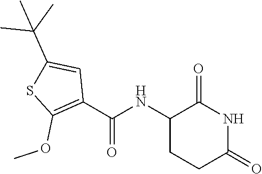 [SMILES: COc1sc(C(C)(C)C)cc1C(=O)NC1CCC(=O)NC1=O] |
| 87 | 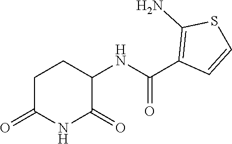 [SMILES: Nc1sccc1C(=O)NC1CCC(=O)NC1=O] |
| 88 | 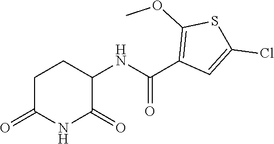 [SMILES: COc1sc(Cl)cc1C(=O)NC1CCC(=O)NC1=O] |
| 89 | 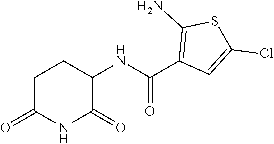 [SMILES: Nc1sc(Cl)cc1C(=O)NC1CCC(=O)NC1=O] |
| 90 | 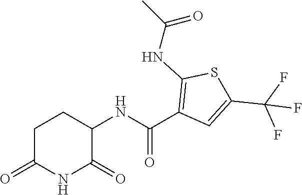 [SMILES: CC(=O)Nc1sc(C(F)(F)F)cc1C(=O)NC1CCC(=O)NC1=O] |
| 91 | 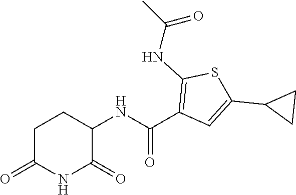 [SMILES: CC(=O)Nc1sc(C2CC2)cc1C(=O)NC1CCC(=O)NC1=O] |
| 92 | 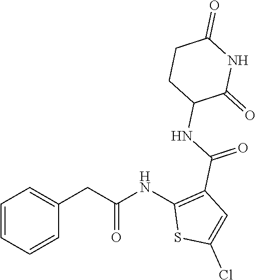 [SMILES: O=C1CCC(NC(=O)c2cc(Cl)sc2NC(=O)Cc2ccccc2)C(=O)N1] |
| 93 | 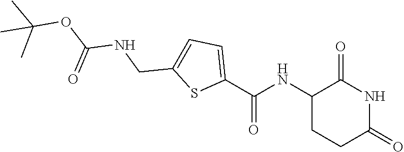 [SMILES: CC(C)(C)OC(=O)NCc1ccc(C(=O)NC2CCC(=O)NC2=O)s1] |
| 94 | 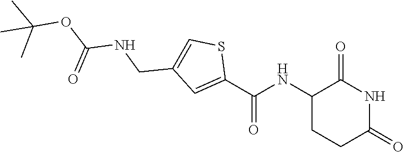 [SMILES: CC(C)(C)OC(=O)NCc1csc(C(=O)NC2CCC(=O)NC2=O)c1] |
| 95 | 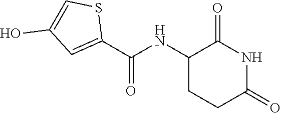 [SMILES: O=C1CCC(NC(=O)c2cc(O)cs2)C(=O)N1] |
| 96 | 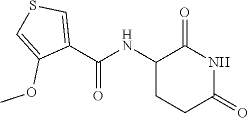 [SMILES: COc1cscc1C(=O)NC1CCC(=O)NC1=O] |
| 97 | 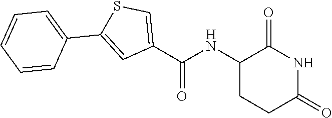 [SMILES: O=C1CCC(NC(=O)c2csc(-c3ccccc3)c2)C(=O)N1] |
| 98 | 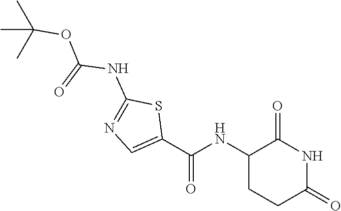 [SMILES: CC(C)(C)OC(=O)Nc1ncc(C(=O)NC2CCC(=O)NC2=O)s1] |
| 99 | 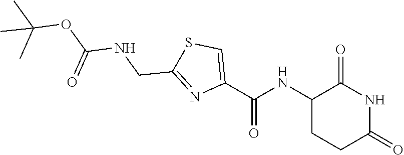 [SMILES: CC(C)(C)OC(=O)NCc1nc(C(=O)NC2CCC(=O)NC2=O)cs1] |
| 100 | 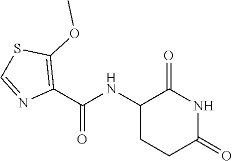 [SMILES: COc1scnc1C(=O)NC1CCC(=O)NC1=O] |
| 101 | 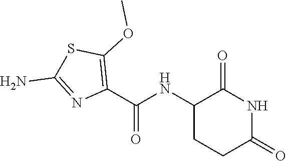 [SMILES: COc1sc(N)nc1C(=O)NC1CCC(=O)NC1=O] |
| 102 | 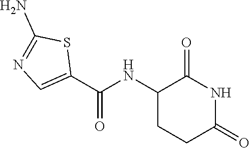 [SMILES: Nc1ncc(C(=O)NC2CCC(=O)NC2=O)s1] |

Example ligase ligand moieties of Formula (IIa) and Formula (IIb) are shown in Table 5 below. Compounds 103, 106 and 107 could be modified to allow attachment to the [linker] (e.g. by C—H bond activation).

TABLE 5

| Compound number | Structure |
|---|---|
| 103 | 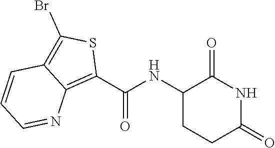 [SMILES: O=C1CCC(NC(=O)c2sc(Br)c3cccnc23)C(=O)N1] |
| 104 | 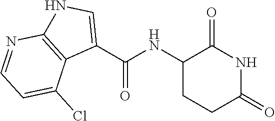 [SMILES: O=C1CCC(NC(=O)c2c[nH]c3nccc(Cl)c23)C(=O)N1] |
| 105 | 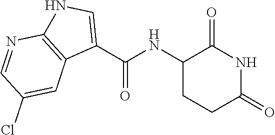 [SMILES: O=C1CCC(NC(=O)c2c[nH]c3ncc(Cl)cc23)C(=O)N1] |
| 106 | 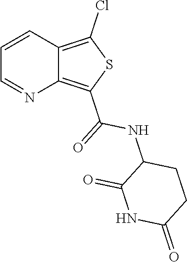 [SMILES: O=C1CCC(NC(=O)c2sc(Cl)c3cccnc23)C(=O)N1] |
| 107 | 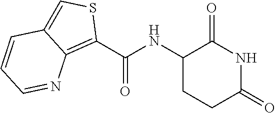 [SMILES: O=C1CCC(NC(=O)c2scc3cccnc23)C(=O)N1] |

In some embodiments of Formulas (Va), (Vb), (IIa) and (IIb) the

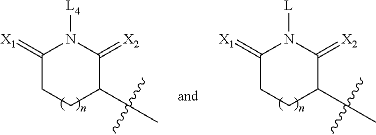

moieties may be replaced with one of the following moieties:

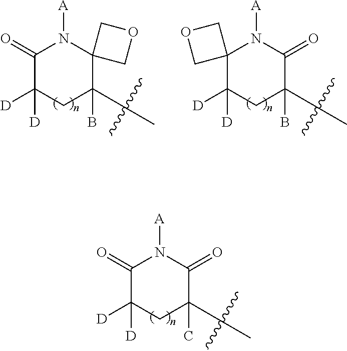

wherein
A is, hydrogen, alkyl, alkenyl, benzyl, aryl, heteroaryl, haloalkyl, haloalkenyl, —CH₂OC(O)^{t}Bu, —CH₂C(O)OR²⁷, —C(O)R²⁷, —C(O)OR²⁷, —C(O)NH₂, —C(O)NHR²⁷, —C(O)NR²⁷², —OR²⁷, —NR²⁷², —S(O)₂R²⁷ or P(O)(OR²⁷)(OR²⁷), wherein each R²⁷ is independently hydrogen, alkyl, cycloalkyl, alkenyl, cycloalkenyl, aryl, heteroaryl, or benzyl,
B is hydrogen, deuterium or alkyl,
C is hydrogen, deuterium or alkyl,
each D is independently deuterium or hydrogen,
   as shown, for example, in the compounds below:

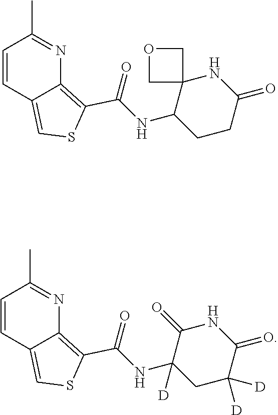 [SMILES: Cc1ccc2csc(C(=O)NC3CCC(=O)NC34COC4)c2n1 ; [2H]C1([2H])CC([2H])(NC(=O)c2scc3ccc(C)nc23)C(=O)NC1=O]

### Linkers

In the compounds of the present invention, the [linker] has the formula R¹⁴—R¹⁵—R¹⁶—R¹⁷—R¹⁸ wherein
R¹⁴ is —C₁₋₆alkyl, —C₂₋₆alkenyl, —C₂₋₆alkynyl, C₁₋₆ alkyl-N(C₁₋₆ alkyl)-, —C(O)—, —SO₂— or is absent
R¹⁵ is cycloalkyl, heterocycloalkyl, aryl, heteroaryl, —C₁₋₆ alkyl-NH—, C₁₋₆ alkyl-N(C₁₋₆ alkyl)-, -cycloalkyl-NH—, heterocycloalkyl-NH—, or is absent
R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, (C₂H₄—O)ₓ, (C₃H₆—O)ₓ, or is absent
x is 1-10
y is 2-10
R¹⁸ is —C₁₋₆ alkyl, heterocycloalkyl, or is absent
   wherein at least one of R¹⁴-R¹⁸ is present.

Linkers as used in the compounds of the present invention may be synthesized according to standard methods.

Most of the alkyl and polyethylene glycol (PEG) linkers were commercially available, or prepared due to procedures described in literature.

Examples of commercially available linkers include:

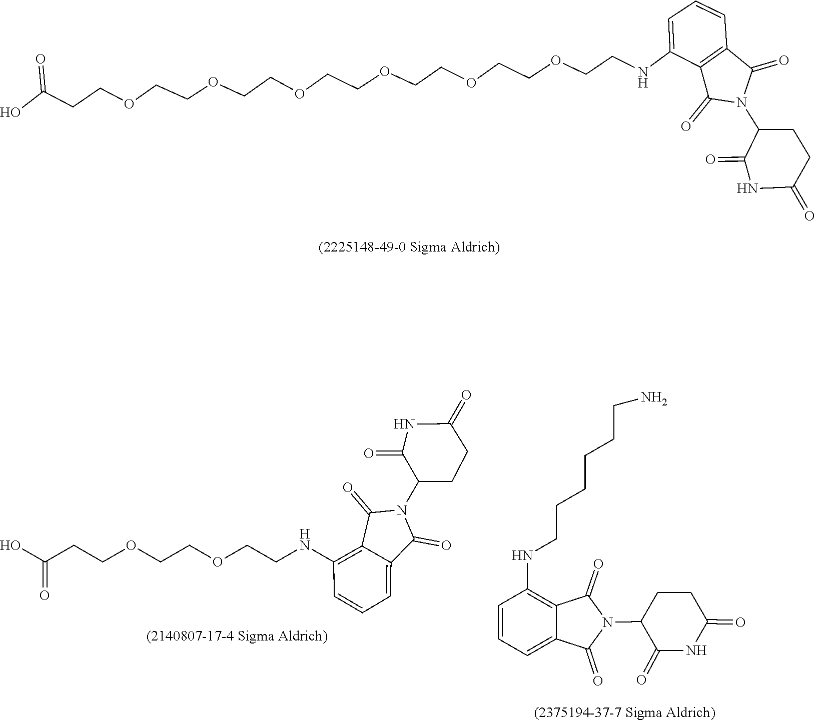 [SMILES: O=C(O)CCOCCOCCOCCOCCOCCOCCNc1cccc2c1C(=O)N(C1CCC(=O)NC1=O)C2=O ; O=C(O)CCOCCOCCNc1cccc2c1C(=O)N(C1CCC(=O)NC1=O)C2=O ; NCCCCCCNc1cccc2c1C(=O)N(C1CCC(=O)NC1=O)C2=O]

Synthesis of linkers which are not commercially available are described in the examples, e.g.: In the synthesis of 227:

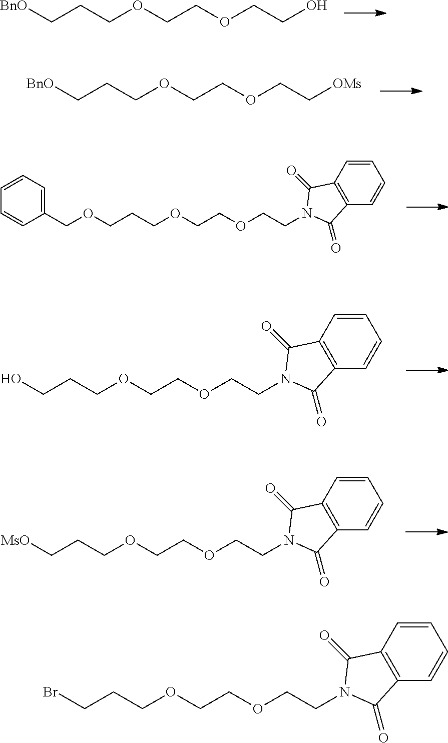 [SMILES: OCCOCCOCCCOCc1ccccc1 ; CS(=O)(=O)OCCOCCOCCCOCc1ccccc1 ; O=C1c2ccccc2C(=O)N1CCOCCOCCCOCc1ccccc1 ; O=C1c2ccccc2C(=O)N1CCOCCOCCCO ; CS(=O)(=O)OCCCOCCOCCN1C(=O)c2ccccc2C1=O ; O=C1c2ccccc2C(=O)N1CCOCCOCCCBr]

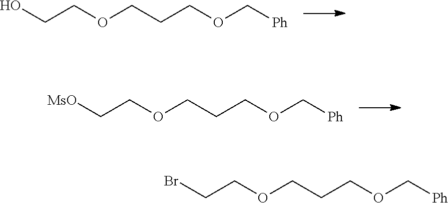 [SMILES: OCCOCCCOCc1ccccc1 ; CS(=O)(=O)OCCOCCCOCc1ccccc1 ; BrCCOCCCOCc1ccccc1]

Also:

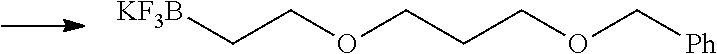

Linkers containing modifications of piperazine were prepared according to the following scheme:

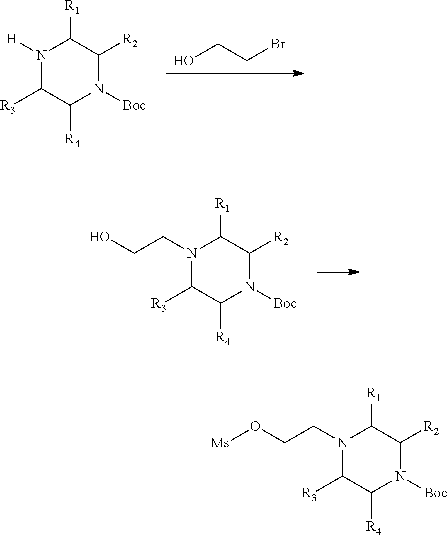

Examples of linkers which can be used in the compounds of the present invention include:

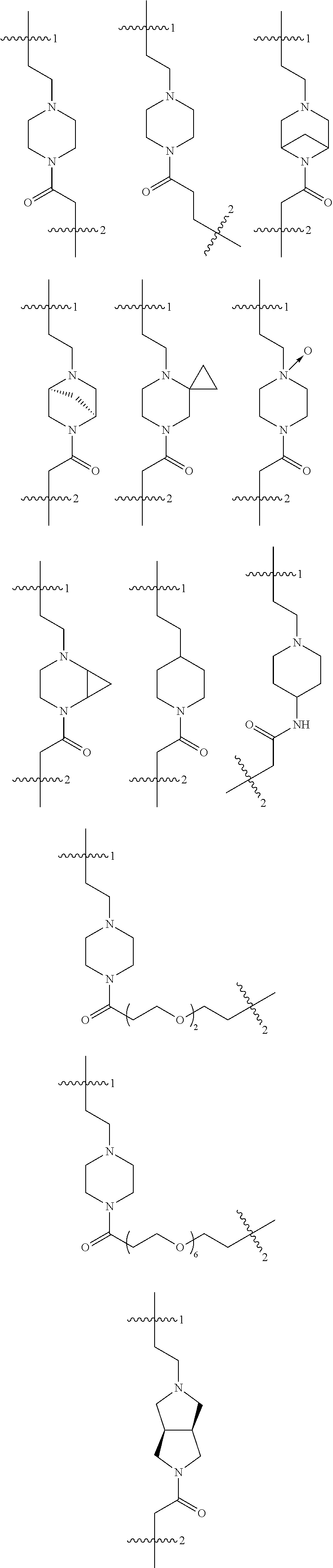

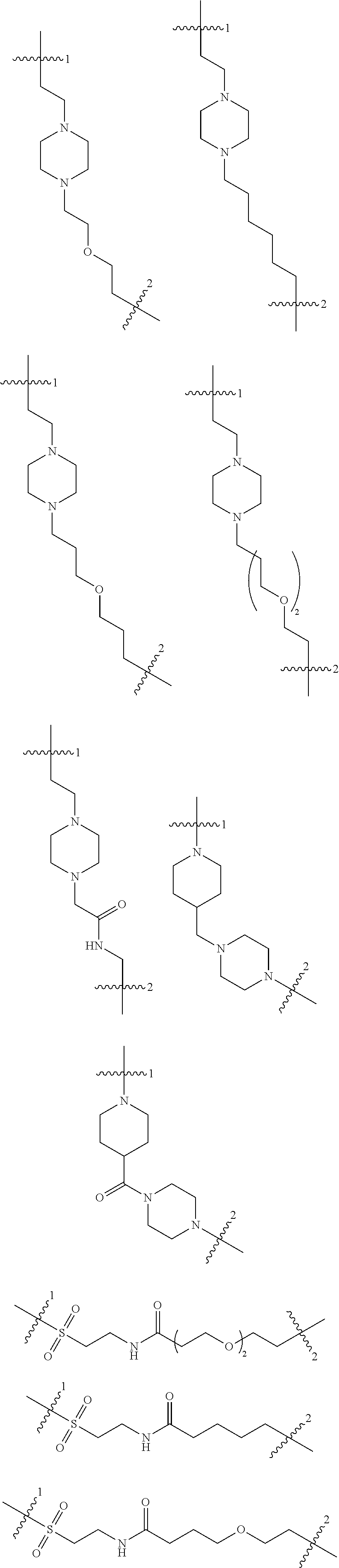

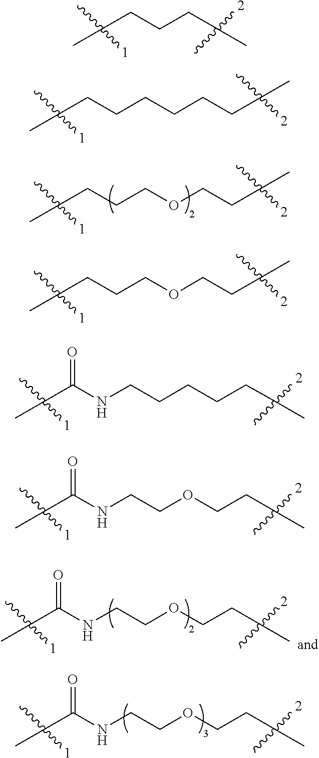

wherein
[symbol] indicates attachment to [MCL-1 ligand moiety] and
[symbol] indicates attachment to [ligase ligand moiety].

### MCL-1 Ligand Moieties

In the compounds of the present invention, the [MCL-1 ligand moiety] is a compound of Formula (A), Formula (B) or Formula (C)

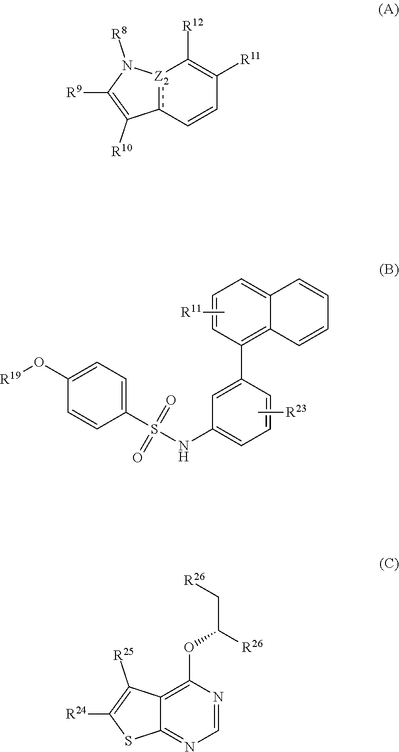

as described above.

Examples of MCL-1 ligand moieties which may be used in the compounds of the present invention include:

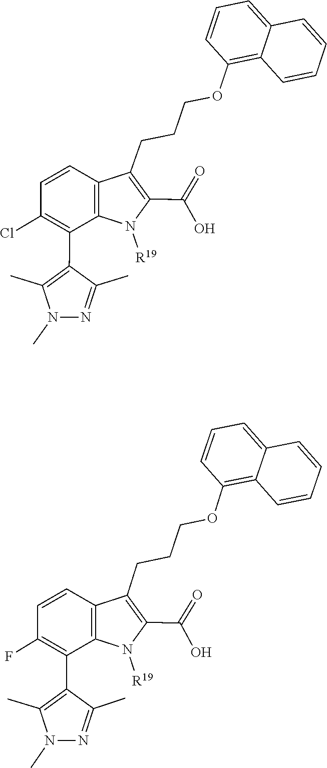

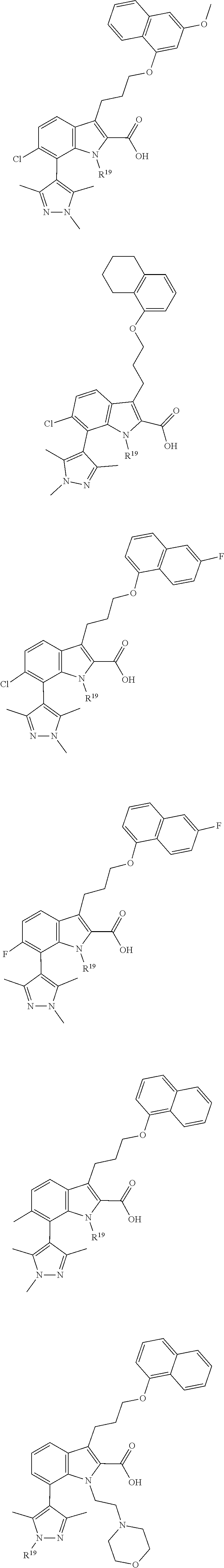

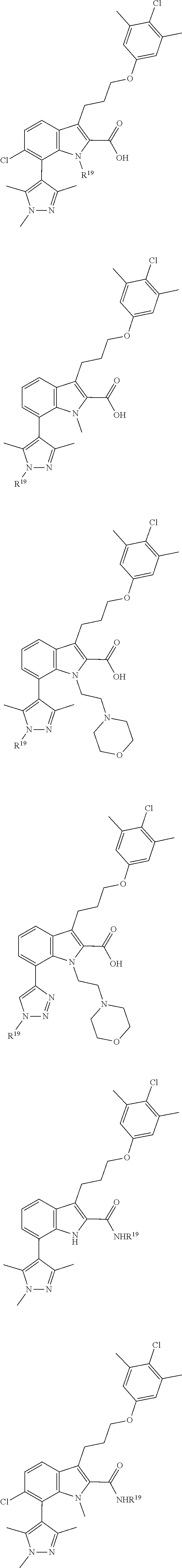

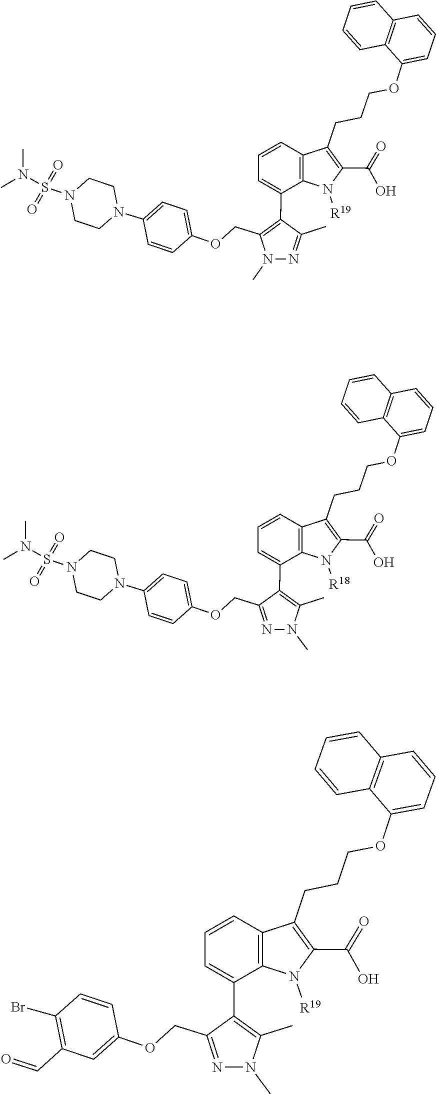

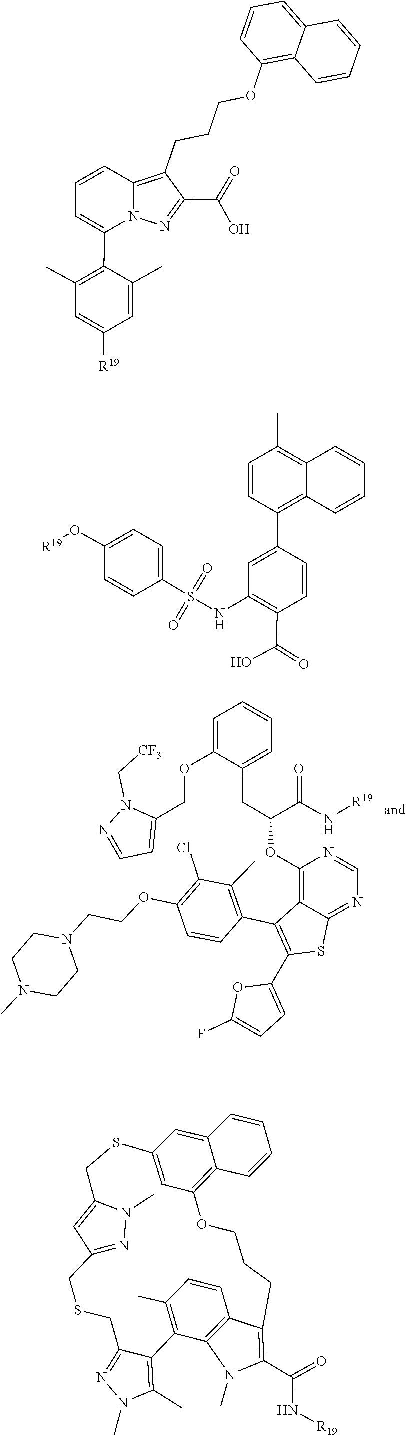

### EXAMPLES

There are a number of ways in which the bifunctional compounds [MCL-1 ligand moiety]-[linker]-[ligase ligand moiety] of the present application may be synthesized:

1. [Mcl-1 Ligand Moiety] is Coupled with linkerA, Followed by Coupling with [Ligase Ligand Moiety]-linkerB

[Mcl-1 ligand moiety]-linkerA+[ligase ligand moiety]-linkerB

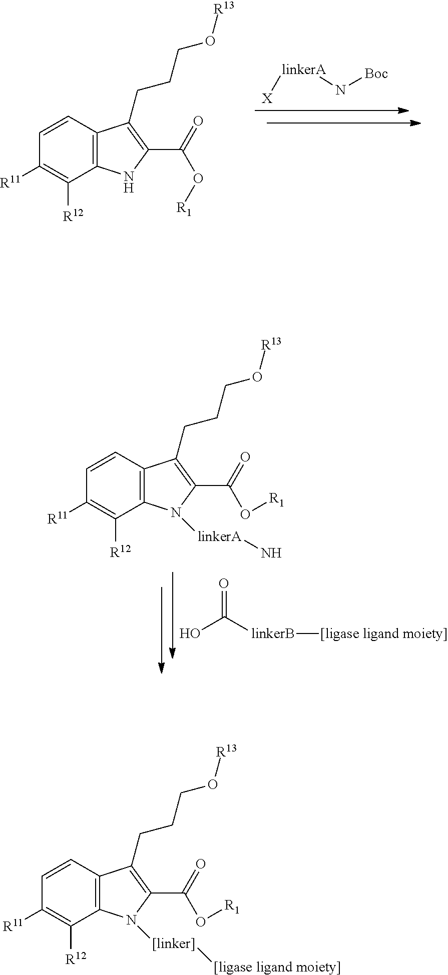

wherein R₁ is —H, —C₁-C₆alkyl, or —NH₂; X is halogen or OMs, OTs; linkerA-N-Boc corresponds to linkerA terminating with a Boc-protected primary or secondary amine; linkerA-NH corresponds to linkerA terminating with a primary or secondary amine; and R¹¹-R¹³ and [linker] are as defined herein; wherein [linker] is formed in the above synthesis by the reaction of -linkerA-NHR^{w} with -linkerB—COOH.

Examples of this method are set out below:

(a) Attachment of Linker Via R⁸ of [MCL-1 Ligand Moiety], and Coupling of linkerA and linkerB Via an Amide Bond:

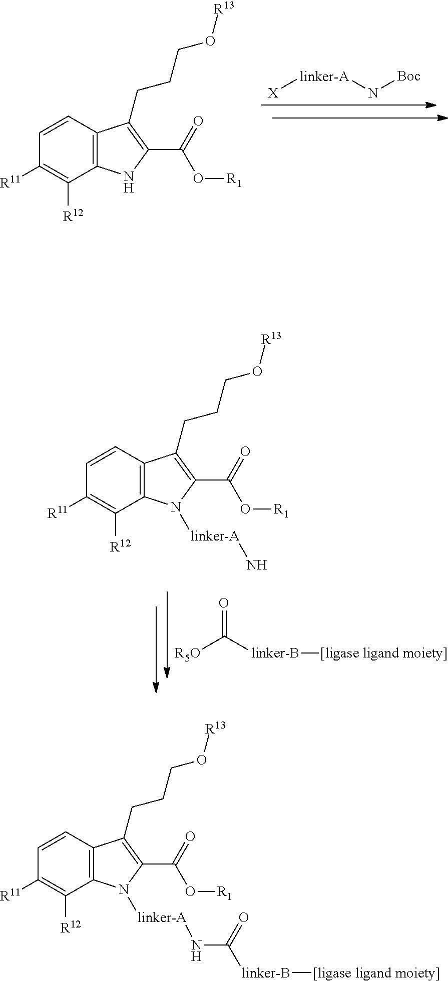

wherein R₁ is —H, —C₁-C₆alkyl, or —NH₂; X is halogen or OMs, OTs; linkerA-N-Boc corresponds to linkerA terminating with a Boc-protected primary or secondary amine; linkerA-NH corresponds to linkerA terminating with a primary or secondary amine; R₅ is succinimidyl or pentafluorophenyl; and R¹¹-R¹³ are as defined herein; and wherein linkerA-NHC(O)-linkerB corresponds to [linker].

This method was used for the synthesis of 201, 203, 204, 205, 206, 207, 211, 208, 210, 209, 214, 216, 213, 215, 217, 233, 241, 245, 248, 249 and 251, below.

(b) Attachment of Linker Via R⁸ of [MCL-1 Ligand Moiety], and Coupling of linkerA and linkerB by Alkylation or Reductive Amination

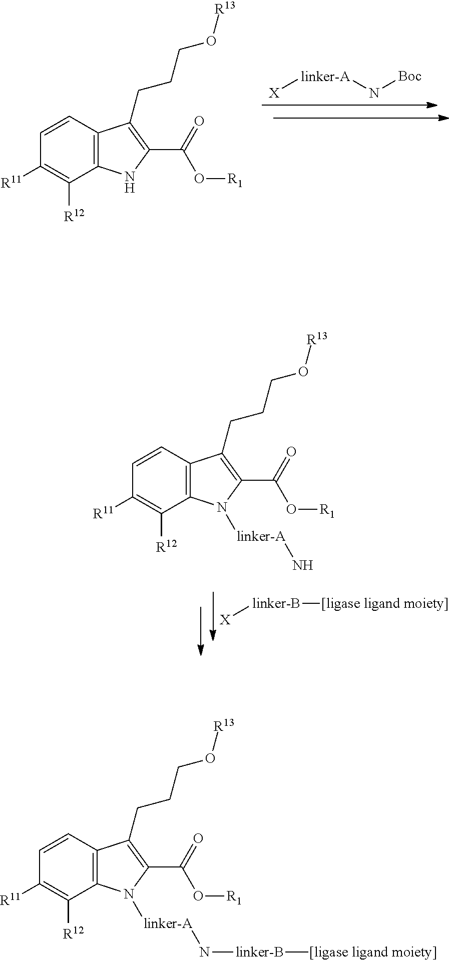

wherein R₁ is —H, —C₁-C₆alkyl, or —NH₂; X is halogen or OMs, OTs; linkerA-N-Boc corresponds to linkerA terminating with a Boc-protected primary or secondary amine; linkerA-NH corresponds to linkerA terminating with a primary or secondary amine; and R¹¹-R¹³ are as defined herein; and wherein linkerA-N-linkerB corresponds to [linker].

This method was used for the synthesis of 233, 234, 235, 236, 237 and 240, below.

(c) Attachment of Linker Via R⁹ of [MCL-1 Ligand Moiety], and Coupling of linkerA and linkerB Via an Amide Bond:

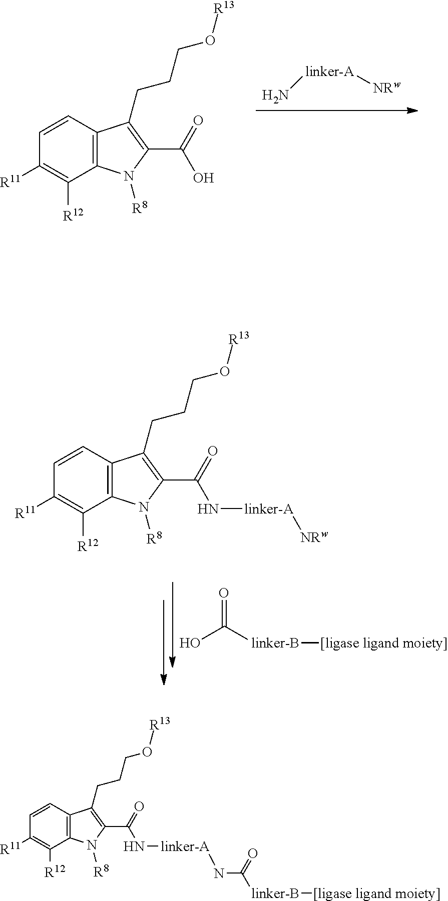

wherein linkerA-N—R^{w} corresponds to linkerA terminating with an R^{w}-protected primary or secondary amine; R⁸ and R¹¹-R¹³ are as defined herein; and wherein linkerA-NC(O)-linkerB corresponds to [linker].

This method was used for the synthesis of 242, 243 and 244, below.

2. [Mcl-1 Ligand Moiety] is Coupled with linkerA, Followed by Coupling with Linker B, Followed Coupling with [Ligase Ligand Moiety]

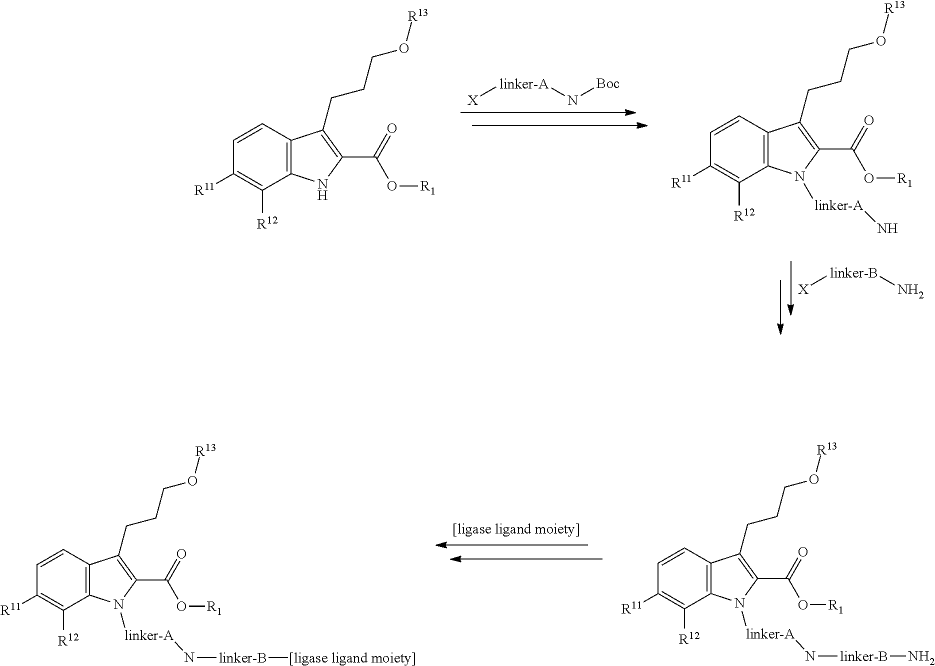

wherein R₁ is —H, —C₁-C₆alkyl, or —NH₂; X is halogen or OMs, OTs; linkerA-N-Boc corresponds to linkerA terminating with a Boc-protected primary or secondary amine; linkerA-NH corresponds to linkerA terminating with a primary or secondary amine; and R¹¹-R¹³ are as defined herein; and wherein linkerA-N-linkerB corresponds to [linker].

This method was used for the synthesis of 247, below.

3. [Mcl-1 Ligand Moiety]-[Linker] is Coupled with [Ligase Ligand Moiety]

[Mcl-1 ligand moiety]-[linker]+[ligase ligand moiety]

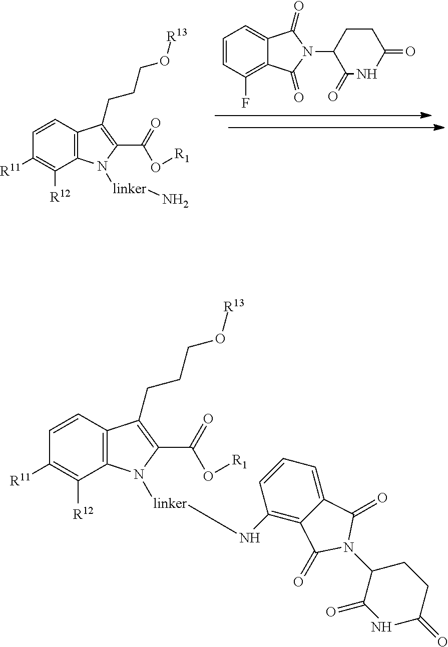

wherein R₁ is —H, —C₁-C₆alkyl, or —NH₂; and R¹¹-R¹³ are as defined herein.

Examples of this method are set out below:

### (i) (as Used for the Synthesis of 227, 228, 229, 230 and 232):

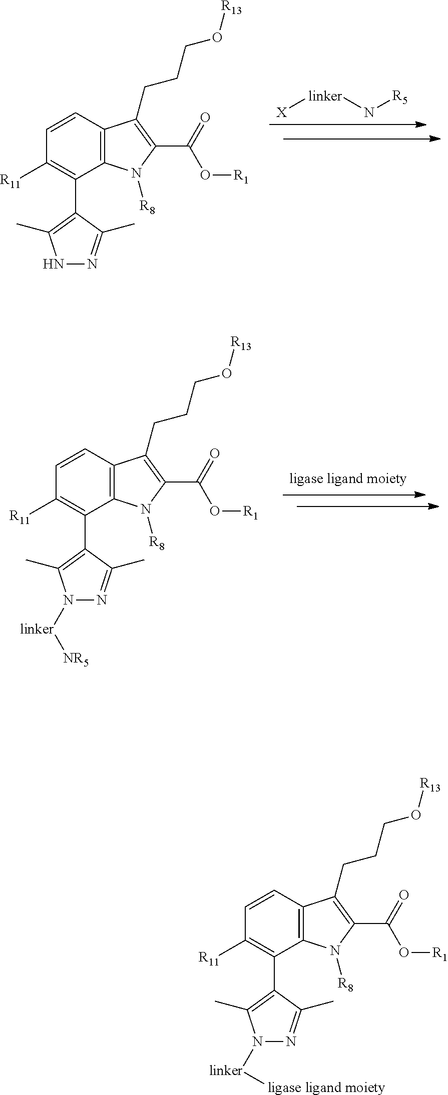

wherein R₁ is —H, —C₁-C₆alkyl, or —NH₂; R⁸, R¹¹ and R¹³ are as defined herein; and wherein linker-N—R₅ corresponds to [linker] terminating with an R₅-protected primary or secondary amine.

### (ii) (as Used for the Synthesis of 253, 254, 255 and 256)

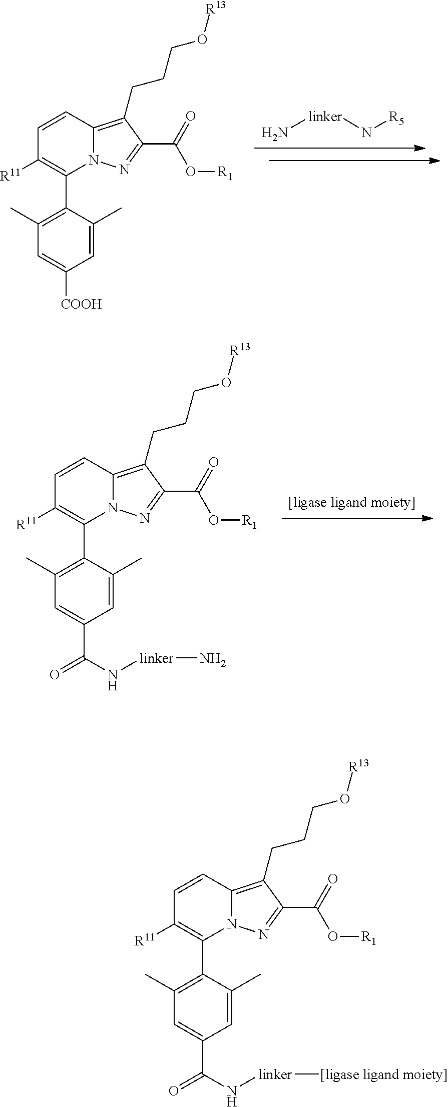

wherein R₁ is —H, —C₁-C₆alkyl, or —NH₂; R¹¹ and R¹³ are as defined herein; and wherein linker-N—R₅ corresponds to [linker] terminating with an R₅-protected primary or secondary amine.
(iii) (as Used for the Synthesis of 238)

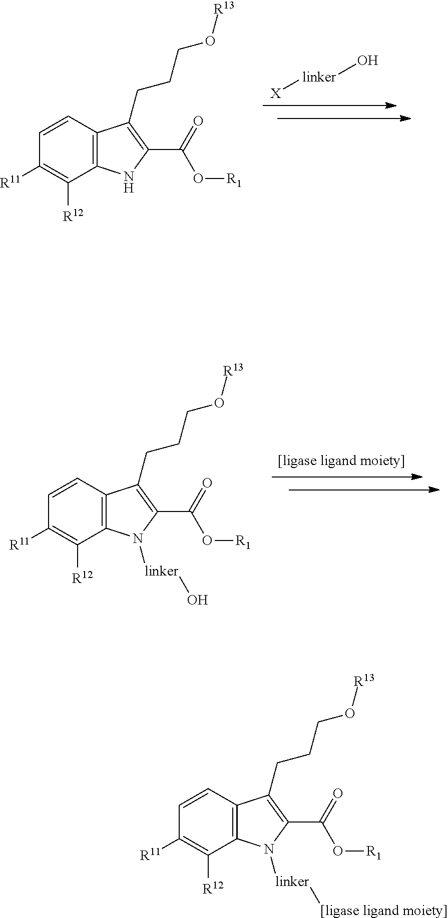

wherein R₁ is —H, —C₁-C₆alkyl, or —NH₂; and R¹¹-R¹³ are as defined herein.
4. [Mcl-1 Ligand Moiety] is Coupled with [Linker]-[Ligase Ligand Moiety]

[Mcl-1 ligand moiety]+[linker]-[ligase ligand moiety]

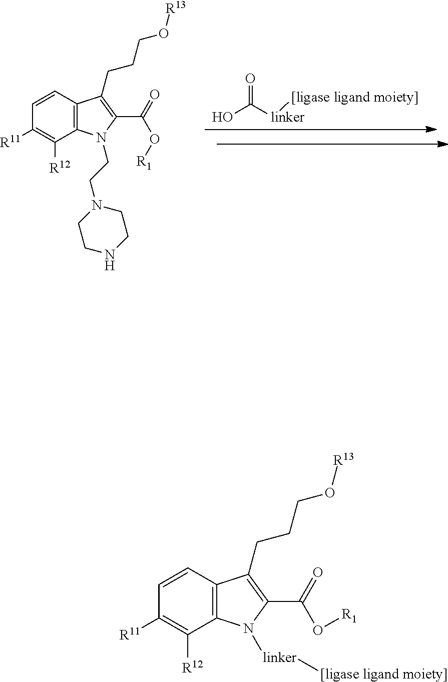

wherein R₁ is —H, —C₁-C₆alkyl, or —NH₂; and R¹¹-R¹³ are as defined herein.

Examples of this method are set out below:

### (i) (as Used for the Synthesis of 252)

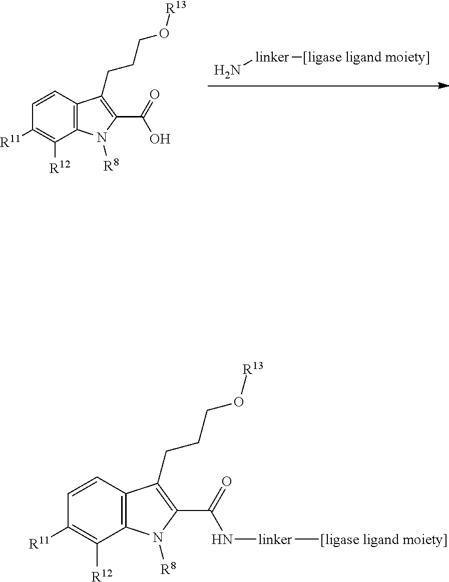

wherein R⁸, R¹¹ and R¹³ are as defined herein.

### (ii) (as Used for the Synthesis of 231)

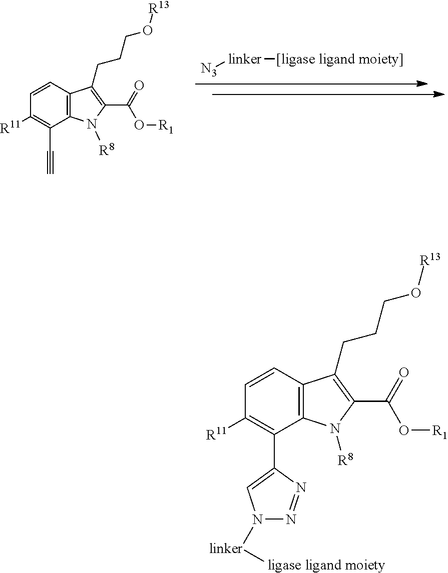

wherein R₁ is —H, —C₁-C₆alkyl, or —NH₂; and R⁸, R¹¹ and R¹³ are as defined herein.
5. [Mcl-1 Ligand Moiety]-[Linker]-R^{v} is Coupled with 3-Aminopiperidine-2,6-Dione

[Mcl-1 ligand moiety]-[linker]-R^{v}+3-Aminopiperidine-2,6-dione

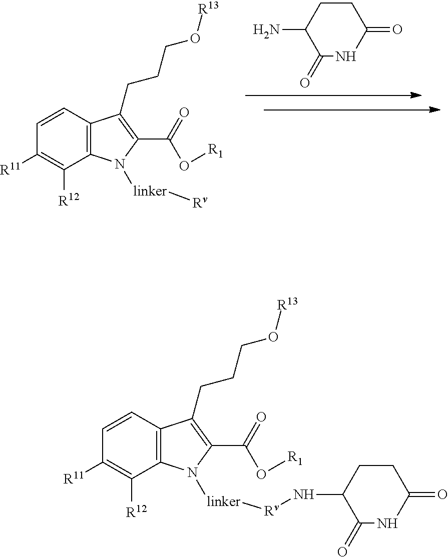

wherein R₁ is —H, —C₁-C₆alkyl, or —NH₂; R¹¹-R¹³ are as defined herein; and R^{v} is -T-R^{x}, -T-R^{y},

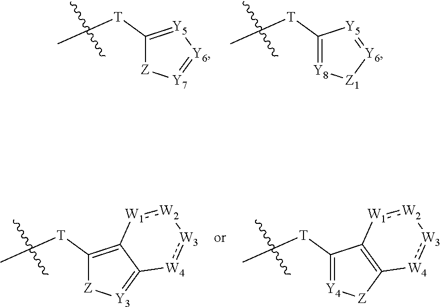

Examples of this method are set out below:

### (i) (as Used for the Synthesis of 263, 264, 265, 266 and 267)

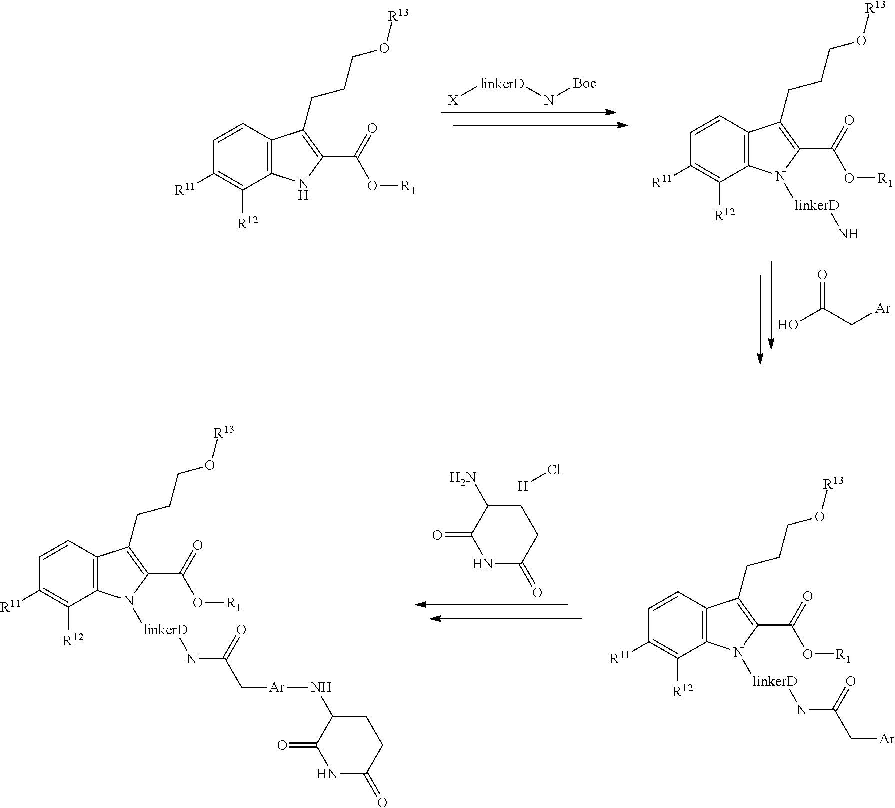

wherein R₁ is —H, —C₁-C₆alkyl, or —NH₂; R¹¹-R¹³ are as defined herein; linker-N-Boc corresponds to [linker] terminating with a Boc-protected primary or secondary amine; linkerD-NC(O)CH₂-corresponds to [linker] as defined herein; and Ar is -T-R^{x}, -T-R^{y},

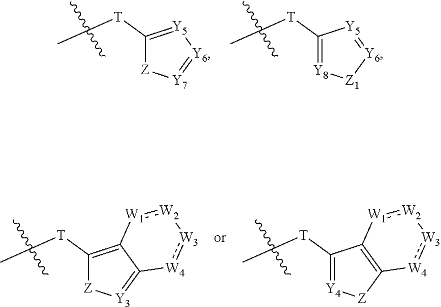

(ii)

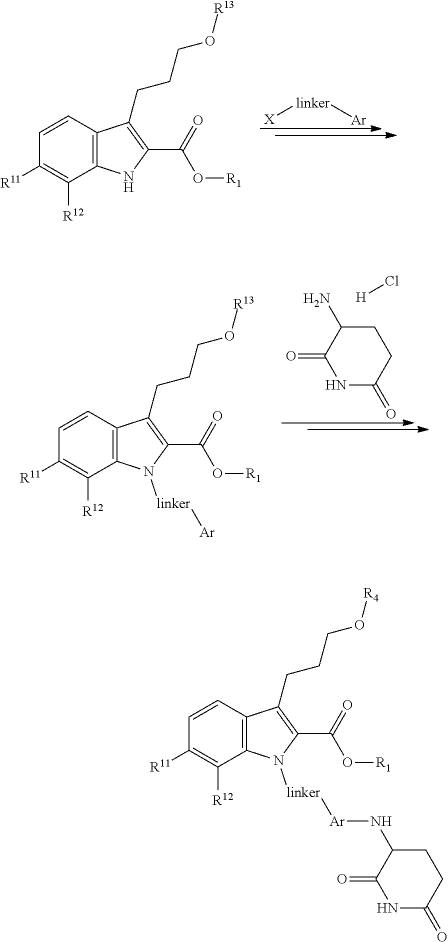

wherein R₁ is —H, —C₁-C₆alkyl, or —NH₂; R¹¹-R¹³ are as defined herein; and Ar is -T-R^{x}, -T-R^{y},

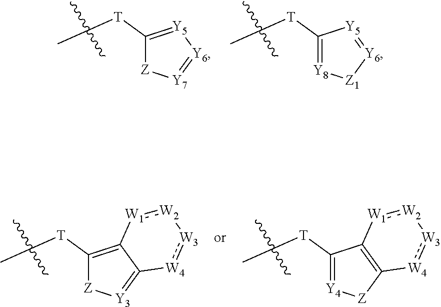

The bifunctional compounds of the present invention were prepared as follows:

### Example 1: 6-chloro-3-(3-(4-chloro-3,5-dimethylphenoxy)propyl)-1-(2-(4-(2-((2-(2,6-dioxopiperidin-3-yl)-1,3-dioxoisoindolin-4-yl)oxy)acetyl)piperazin-1-yl)ethyl)-7-(1,3,5-trimethyl-1H-pyrazol-4-yl)-1H-indole-2-carboxylic acid (201)

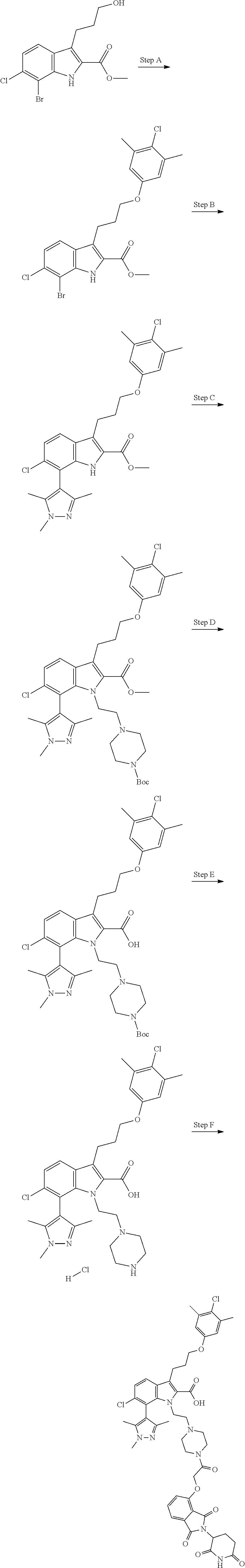

### Step A

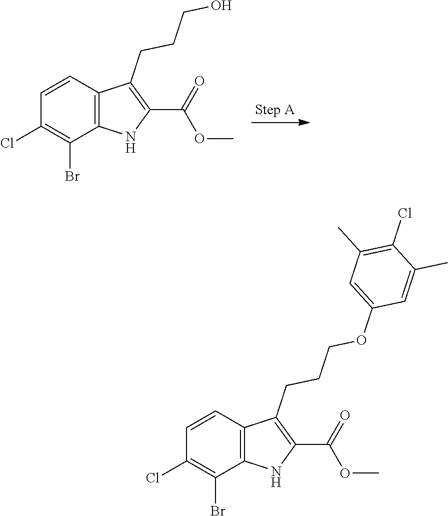 [SMILES: COC(=O)c1[nH]c2c(Br)c(Cl)ccc2c1CCCO ; COC(=O)c1[nH]c2c(Br)c(Cl)ccc2c1CCCOc1cc(C)c(Cl)c(C)c1]

To a stirred solution of methyl 7-bromo-6-chloro-3-(3-hydroxypropyl)-1H-indole-2-carboxylate (20 g, 57.7 mmol), 4-chloro-3,5-dimethylphenol (10.84 g, 69.2 mmol), PPh₃ (18.2 g, 69.4 mmol) in THE (470 mL) was added in portions (E)-N-{[(tert-butoxy)carbonyl]imino}(tert-butoxy)formamide (16 g, 69.5 mmol). The reaction mixture was stirred at room temperature overnight. Then the solvent was removed under reduced pressure and the residue was purified by flash chromatography to afford methyl 7-bromo-6-chloro-3-(3-(4-chloro-3,5-dimethylphenoxy)propyl)-1H-indole-2-carboxylate (13.8 g, 28.4 mmol, 49%).

### Step B

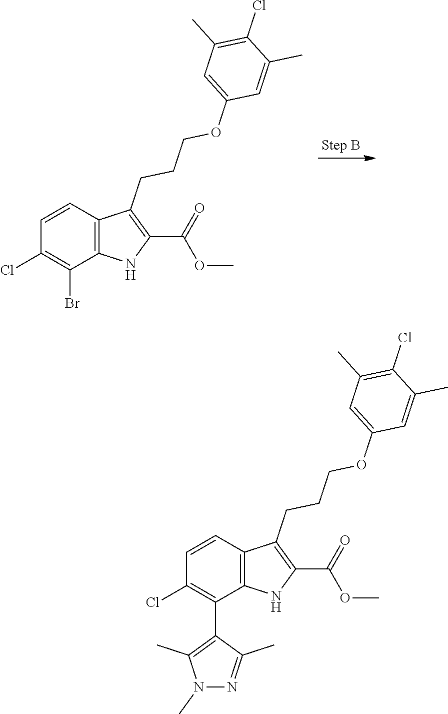

To a stirred solution of methyl 7-bromo-6-chloro-3-(3-(4-chloro-3,5-dimethylphenoxy)propyl)-1H-indole-2-carboxylate (13.8 g, 28.4 mmol) and 1,3,5-trimethyl-4-(4,4,5,5-tetramethyl-1,3,2-dioxaborolan-2-yl)-1H-pyrazole (7.4 g, 31.4 mmol) in dioxane (200 mL) was added aqueous solution of K₂CO₃ (5.5 g, 40 mmol in 30 mL water). The reaction mixture was degassed and Pd(PPh₃)₄ (2 g, 1.7 mmol) was added under argon atmosphere. The reaction mixture was heated at 80° C. for 24 h. New portion of 1,3,5-trimethyl-4-(4,4,5,5-tetramethyl-1,3,2-dioxaborolan-2-yl)-1H-pyrazole (7.4 g, 31.4 mmol), K₂CO₃ (5.5 g, 40 mmol) and Pd(PPh₃)₄ (4 g, 3.4 mmol) were added and the reaction was continued for 2 days. Then the solid was filtered, washed with EtOAc, and the filtrate was concentrated. The residue was dissolved in CH₂Cl₂, washed with water and purified by flash chromatography to afford methyl 6-chloro-3-(3-(4-chloro-3,5-dimethylphenoxy)propyl)-7-(1,3,5-trimethyl-1H-pyrazol-4-yl)-1H-indole-2-carboxylate (2.20 g, 4.28 mmol, 15%).

### Step C

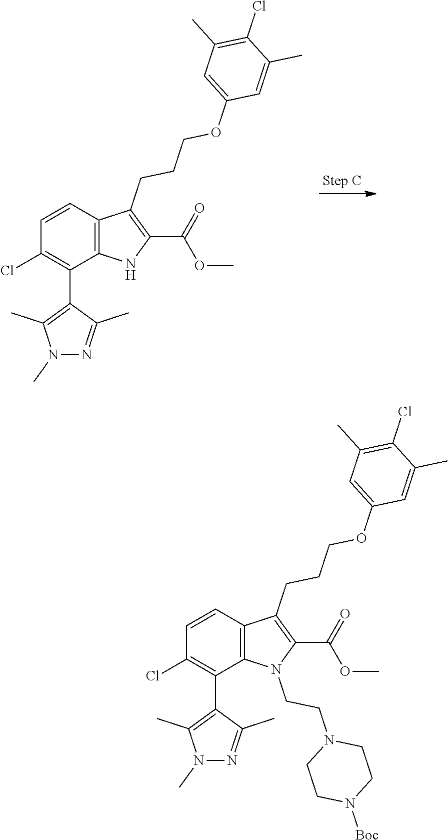

Methyl 6-chloro-3-(3-(4-chloro-3,5-dimethylphenoxy)propyl)-7-(1,3,5-trimethyl-1H-pyrazol-4-yl)-1H-indole-2-carboxylate (200.0 mg, 0.389 mmol) was dissolved in DMF (3.9 mL) and Cs₂CO₃ was added followed by tert-butyl 4-(2chloroethyl)piperazine-1-carboxylate (97.7 mg, 0.393 mmol). Mixture was heated in 80° C. for overnight. Solvent was evaporated, EtOAc and brine were added, and mixture was extracted with EtOAc. Combined organic layers were dried over MgSO₄, filtered and concentrated in vacuum to give methyl 1-(2-{4-[(tert-butoxy)carbonyl]piperazin-1-yl}ethyl)-6-chloro-3-[3-(4-chloro-3,5-dimethylphenoxy)propyl]-7-(1,3,5-trimethyl-1H-pyrazol-4-yl)-1H-indole-2-carboxylate) (273.0 mg, 0.376 mmol, 96.6%) as white foam.

LCMS (ESI+): m/z 726.2 [M+H]⁺

### Step D

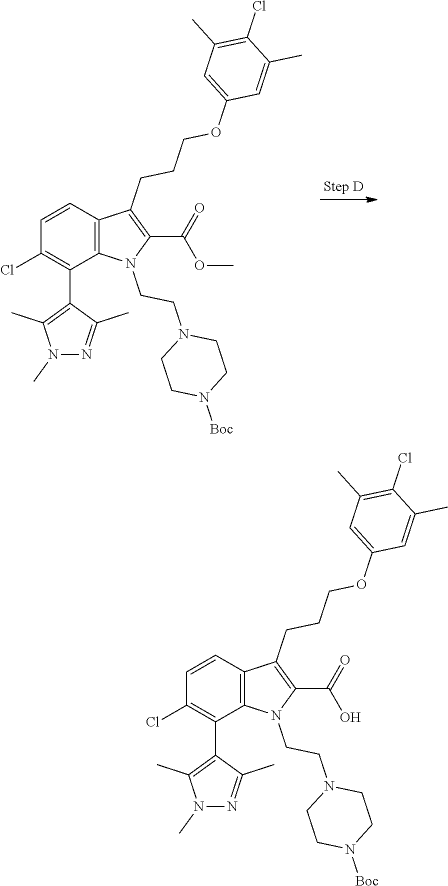

Methyl 1-(2-{4-[(tert-butoxy)carbonyl]piperazin-1-yl}ethyl)-6-chloro-3-[3-(4-chloro-3,5-dimethylphenoxy)propyl]-7-(1,3,5-trimethyl-1H-pyrazol-4-yl)-1H-indole-2-carboxylate (270.0 mg, 0.372 mmol) was dissolved in EtOH (1.9 mL) and water (1.9 mL) and 1M LiOH (1.5 mL, 1.486 mmol) was added. The reaction was allowed to stir overnight. THE (1.9 mL) was added and reaction was heated for overnight in 50° C. The reaction was diluted with water and washed with EtOAc. The water layer was then acidified with 1N HCl to pH 2. The resulting suspension was extracted with DCM. The organic layer was washed with saturated sodium chloride, dried over MgSO₄, filtered, and concentrated. Pure 1-(2-{4-[(tert-butoxy)carbonyl]piperazin-1-yl}ethyl)-6-chloro-3-[3-(4-chloro-3,5-dimethylphenoxy)propyl]-7-(1,3,5-trimethyl-1H-pyrazol-4-yl)-1H-indole-2-carboxylic acid (258.0 mg, 0.344 mmol, 92.6%) was obtained as a white solid.

LCMS (ESI⁺): m/z 712.3 [M+H]⁺

### Step E

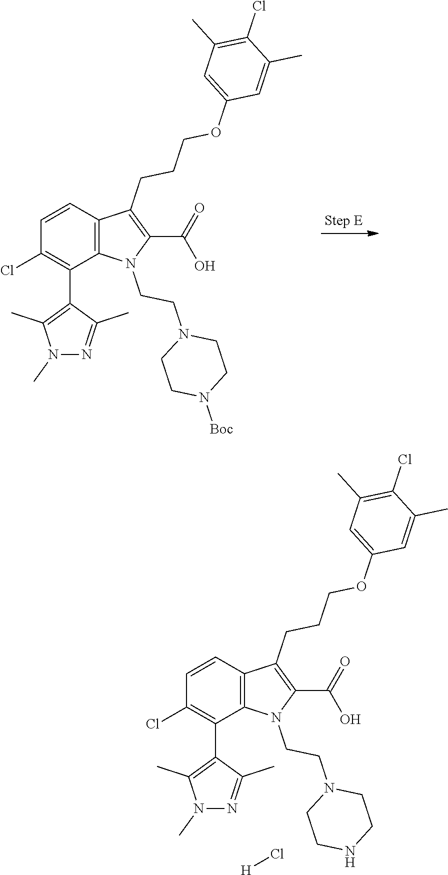

1-(2-{4-[(Tert-butoxy)carbonyl]piperazin-1-yl}ethyl)-6-chloro-3-[3-(4-chloro-3,5-dimethylphenoxy)propyl]-7-(1,3,5-trimethyl-1H-pyrazol-4-yl)-1H-indole-2-carboxylic acid (258.0 mg, 0.362 mmol) was dissolved in THE (3.6 mL) and 4M HCl in dioxane (0.271 mL, 1.086 mmol) was added. Mixture was stirred in room temperature. After 2 days solvents were removed and residue was coevaporated with Et₂O. 6-Chloro-3-[3-(4-chloro-3,5-dimethylphenoxy)propyl]-1-[2-(piperazin-1-yl)ethyl]-7-(1,3,5-trimethyl-1H-pyrazol-4-yl)-1H-indole-2-carboxylic acid hydrochloride (215.0 mg, 0.331 mmol, 91.5%) was obtained as white solid.

LCMS (ESI⁺): m/z 611.4 [M+H]⁺

### Step F

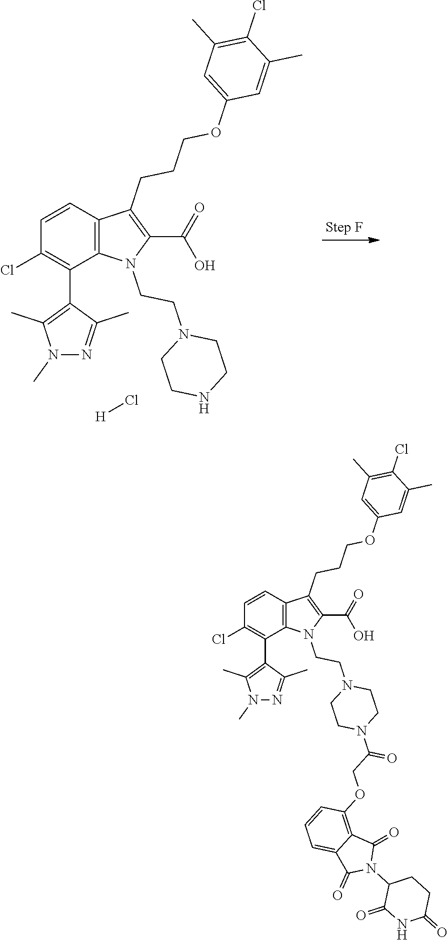

A solution of 2-((2-(2,6-dioxopiperidin-3-yl)-1,3-dioxoisoindolin-4-yl)oxy)acetic acid (25 mg, 0.075 mmol) and CDI (12.2 mg, 0.075 mmol) in DCM (0.6 mL) was stirred for 1 h in 50° C. After this time 6-chloro-3-[3-(4-chloro-3,5-dimethylphenoxy)propyl]-1-[2-(piperazin-1-yl)ethyl]-7-(1,3,5-trimethyl-1H-pyrazol-4-yl)-1H-indole-2-carboxylic acid hydrochloride (38.8 mg, 0.060 mmol), was added and the mixture was allowed to stir under nitrogen for 16 h in room temperature. The reaction mixture diluted with EtOAc, washed successively with cold water (3 times) and brine. Organic layer was dried over Na₂SO₄ and evaporated under reduced pressure. Crude was purified using preparative HPLC (H₂O:MeCN+0.1% FA) to get 6-chloro-3-(3-(4-chloro-3,5-dimethylphenoxy)propyl)-1-(2-(4-(2-((2-(2,6-dioxopiperidin-3-yl)-1,3-dioxoisoindolin-4-yl)oxy)acetyl)piperazin-1-yl)ethyl)-7-(1,3,5-trimethyl-1H-pyrazol-4-yl)-1H-indole-2-carboxylic acid (7.0 mg, 0.008 mmol, 13%) as white solid.

LCMS (ESI+): m/z 926.9 [M+H]⁺

¹H NMR (500 MHz, DMSO) δ 13.32 (s, 1H), 11.09 (s, 1H), 7.76 (dd, J=8.6, 7.3 Hz, 1H), 7.68 (d, J=8.6 Hz, 1H), 7.44 (d, J=7.2 Hz, 1H), 7.29 (d, J=8.6 Hz, 1H), 7.24 (d, J=8.5 Hz, 1H), 6.73 (s, 2H), 5.19-5.00 (m, 3H), 4.35-4.22 (m, 1H), 4.22-4.09 (m, 1H), 3.98 (t, J=6.4 Hz, 2H), 3.75 (s, 3H), 3.12 (t, J=7.4 Hz, 2H), 2.88 (ddd, J=16.8, 13.8, 5.5 Hz, 1H), 2.61-2.52 (m, 3H), 2.26 (s, 7H), 2.18-1.92 (m, 14H), 1.88 (s, 3H).

### Example 2: 6-chloro-3-[3-(4-chloro-3,5-dimethylphenoxy)propyl]-1-{2-(4-(1-{(2-(2,6-dioxopiperidin-3-yl)-1,3-dioxo-2,3-dihydro-1H-isoindol-4-yl]amino}-3,6,9,12,15,18-hexaoxahenicosanoyl)piperazin-1-yl]ethyl}-7-(1,3,5-trimethyl-1H-pyrazol-4-yl)-1H-indole-2-carboxylic acid (203)

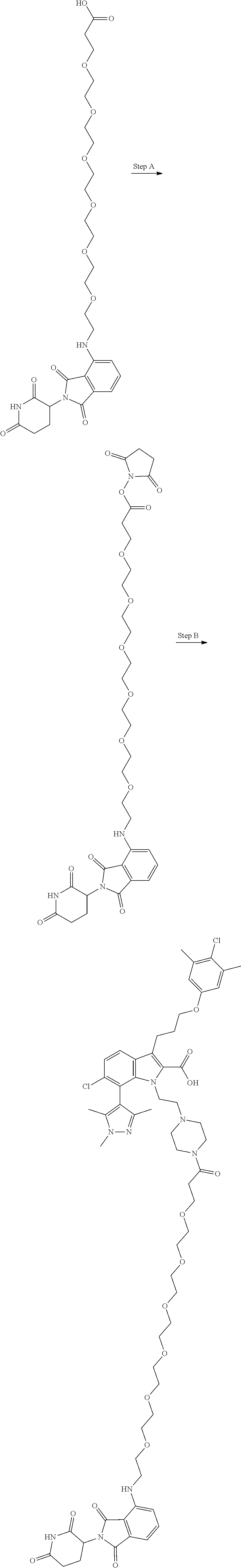 [SMILES: O=C(O)CCOCCOCCOCCOCCOCCOCCNc1cccc2c1C(=O)N(C1CCC(=O)NC1=O)C2=O ; O=C1CCC(N2C(=O)c3cccc(NCCOCCOCCOCCOCCOCCOCCC(=O)ON4C(=O)CCC4=O)c3C2=O)C(=O)N1 ; Cc1cc(OCCCc2c(C(=O)O)n(CCN3CCN(C(=O)CCOCCOCCOCCOCCOCCOCCNc4cccc5c4C(=O)N(C4CCC(=O)NC4=O)C5=O)CC3)c3c(-c4c(C)nn(C)c4C)c(Cl)ccc23)cc(C)c1Cl]

### Step A

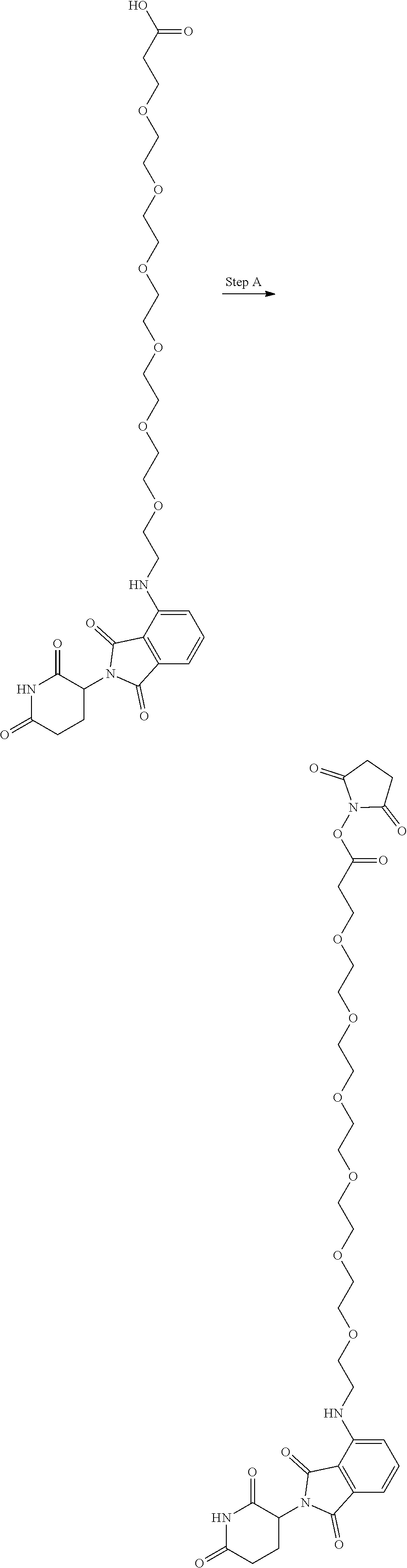 [SMILES: O=C(O)CCOCCOCCOCCOCCOCCOCCNc1cccc2c1C(=O)N(C1CCC(=O)NC1=O)C2=O ; O=C1CCC(N2C(=O)c3cccc(NCCOCCOCCOCCOCCOCCOCCC(=O)ON4C(=O)CCC4=O)c3C2=O)C(=O)N1]

N-hydroxysuccinimide (11.3 mg, 0.098 mmol) was added into a mixture of 1-{[2-(2,6-dioxopiperidin-3-yl)-1,3-dioxo-2,3-dihydro-1H-isoindol-4-yl]amino}-3,6,9,12,15,18-hexaoxahenicosan-21-oic acid (50.0 mg, 0.082 mmol) and DCM (1.6 mL) and the reaction mixture was kept cold at 0° C., DCC (20.3 mg, 0.098 mmol) in 0.5 mL DCM was added slowly and the mixture was stirred at room temperature for 4 hours under argon atmosphere. Solvent was removed under reduced pressure. Desired product was purified using flash chromatography (SiO₂, 10% MeOH in DCM). 2,5-dioxopyrrolidin-1-yl 1-{[2-(2,6-dioxopiperidin-3-yl)-1,3-dioxo-2,3-dihydro-1H-isoindol-4-yl]amino}-3,6,9,12,15,18-hexaoxahenicosan-21-oate (39.0 mg, 0.051 mmol, 61.9%) was obtained as yellow oil.

LCMS (ESI+): m/z 707.4 [M+H]⁺

### Step B

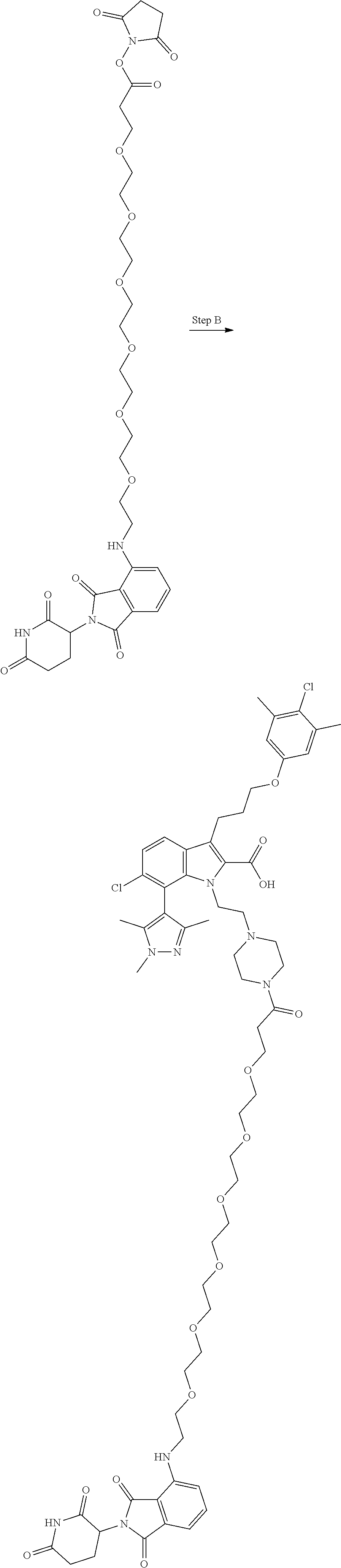 [SMILES: O=C1CCC(N2C(=O)c3cccc(NCCOCCOCCOCCOCCOCCOCCC(=O)ON4C(=O)CCC4=O)c3C2=O)C(=O)N1 ; Cc1cc(OCCCc2c(C(=O)O)n(CCN3CCN(C(=O)CCOCCOCCOCCOCCOCCOCCNc4cccc5c4C(=O)N(C4CCC(=O)NC4=O)C5=O)CC3)c3c(-c4c(C)nn(C)c4C)c(Cl)ccc23)cc(C)c1Cl]

To a stirred solution of 6-chloro-3-[3-(4-chloro-3,5-dimethylphenoxy)propyl]-1-[2-(piperazin-1-yl)ethyl]-7-(1,3,5-trimethyl-1H-pyrazol-4-yl)-1H-indole-2-carboxylic acid hydrochloride (32.1 mg, 0.050 mmol) in DMF (0.495 mL) was added DIPEA (0.035 mL, 0.198 mmol). After stirring for 5 min, the 2,5-dioxopyrrolidin-1-yl 1-{[2-(2,6-dioxopiperidin-3-yl)-1,3-dioxo-2,3-dihydro-1H-isoindol-4-yl]amino}-3,6,9,12,15,18-hexaoxahenicosan-21-oate (35.0 mg, 0.050 mmol) was added and stirred for 16 h at 25° C. Solvents were removed under reduced pressure. Desired product was purified using flash chromatography (SiO₂, 10% MeOH in DCM) and preparative HPLC (H₂O:MeCN+0.1% FA). 6-Chloro-3-[3-(4-chloro-3,5-dimethylphenoxy)propyl]-1-{2-[4-(1-{[2-(2,6-dioxopiperidin-3-yl)-1,3-dioxo-2,3-dihydro-1H-isoindol-4-yl]amino}-3,6,9,12,15,18-hexaoxahenicosanoyl)piperazin-1-yl]ethyl}-7-(1,3,5-trimethyl-1H-pyrazol-4-yl)-1H-indole-2-carboxylic acid (16.0 mg, 0.013 mmol, 27%) was obtained as yellow solid.

LCMS (ESI+): m/z 603.14 [M+2H]²⁺

1H NMR (500 MHz, DMSO) δ 7.66 (d, J=8.6 Hz, 1H), 7.55 (dd, J=8.6, 7.1 Hz, 1H), 7.22 (d, J=8.5 Hz, 1H), 7.09 (d, J=8.6 Hz, 1H), 7.01 (d, J=7.0 Hz, 1H), 6.64 (s, 2H), 5.00 (dd, J=12.9, 5.5 Hz, 1H), 4.25-4.15 (m, 1H), 4.15-4.04 (m, 1H), 3.93 (t, J=6.5 Hz, 2H), 3.58 (t, J=5.4 Hz, 2H), 3.57-3.49 (m, 5H), 3.51-3.38 (m, 20H), 3.33-3.21 (m, 4H), 3.08 (t, J=7.3 Hz, 2H), 2.83 (ddd, J=17.2, 13.9, 5.4 Hz, 1H), 2.58 (ddd, J=17.2, 4.4, 2.5 Hz, 1H), 2.56-2.50 (m, 2H), 2.49-2.40 (m, 4H), 2.21 (s, 6H), 2.10-1.93 (m, 12H), 1.84 (s, 3H).

### Example 3: 6-chloro-1-(2-(4-(2-((2-(2,6-dioxopiperidin-3-yl)-1,3-dioxoisoindolin-4-yl)oxy)acetyl)piperazin-1-yl)ethyl)-3-(3-(naphthalen-1-yloxy)propyl)-7-(1,3,5-trimethyl-1H-pyrazol-4-yl)-1H-indole-2-carboxylic acid (204)

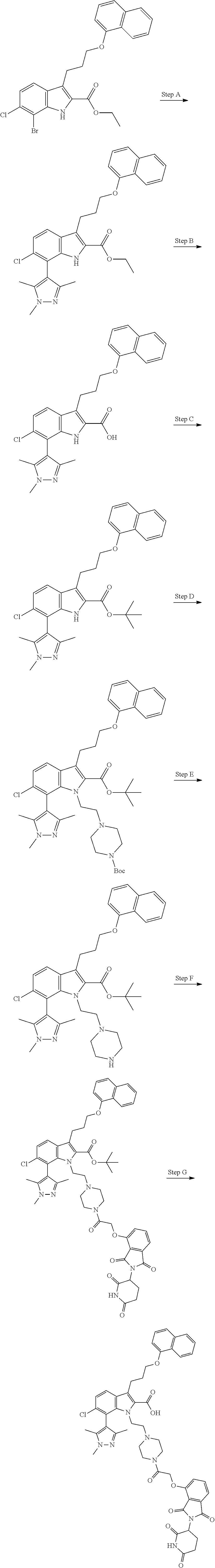

### Step A

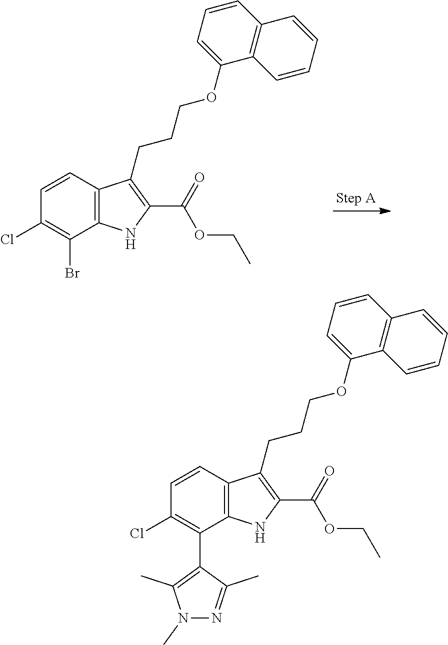 [SMILES: CCOC(=O)c1[nH]c2c(Br)c(Cl)ccc2c1CCCOc1cccc2ccccc12 ; CCOC(=O)c1[nH]c2c(-c3c(C)nn(C)c3C)c(Cl)ccc2c1CCCOc1cccc2ccccc12]

To a stirred solution of ethyl 7-bromo-6-chloro-3-(3-(naphthalen-1-yloxy)propyl)-1H-indole-2-carboxylate (6 g, 12.3 mmol) in dioxane (150 mL) and water (30 mL) were added 1,3,5-trimethyl-4-(4,4,5,5-tetramethyl-1,3,2-dioxaborolan-2-yl)-1H-pyrazole (8.8 g, 37.3 mmol) and K₂CO₃ (4.5 g, 32.6 mmol). The mixture was deoxygenated with argon and to it was added Pd(dppf)Cl₂ (1 g, 1.37 mmol) under argon atmosphere. Then the reaction mixture was heated under reflux for 16 h. After complete consumption of the starting material (monitored by TLC and LCMS) the reaction mixture was filtered through celite pad and the solvents were evaporated under reduced pressure to get the crude material. It was then diluted with EtOAc, washed successively with water and brine, the organic layer was dried over Na₂SO₄ and evaporated under reduced pressure to get the crude compound, which was then purified by flash chromatography (SiO₂, 3% MeOH in DCM) to get ethyl 6-chloro-3-(3-(naphthalen-1-yloxy)propyl)-7-(1,3,5-trimethyl-1H-pyrazol-4-yl)-1H-indole-2-carboxylate (4.2 g, 8.13 mmol, 66%) as brown sticky solid.

LCMS (ESI⁺): m/z 516.5 [M+H]⁺

### Step B

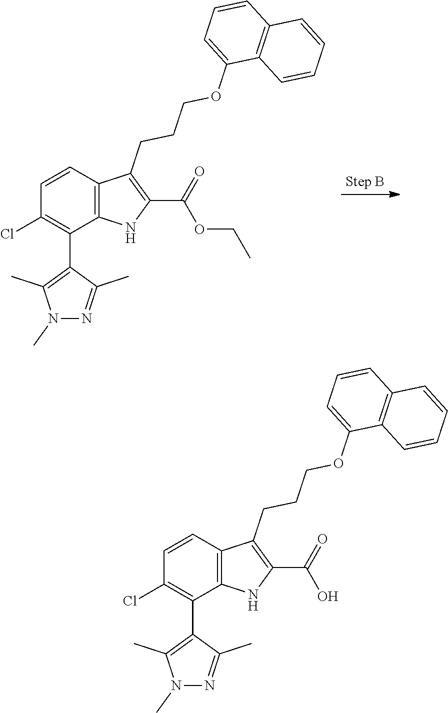 [SMILES: CCOC(=O)c1[nH]c2c(-c3c(C)nn(C)c3C)c(Cl)ccc2c1CCCOc1cccc2ccccc12 ; Cc1nn(C)c(C)c1-c1c(Cl)ccc2c(CCCOc3cccc4ccccc34)c(C(=O)O)[nH]c12]

Ethyl 6-chloro-3-(3-(naphthalen-1-yloxy)propyl)-7-(1,3,5-trimethyl-1H-pyrazol-4-yl)-1H-indole-2-carboxylate (4.2 g, 8.13 mmol) was dissolved in EtOH (100 mL) and a solution of NaOH (1.2 g, 30.0 mmol) in water (20 mL) was added to it. The mixture was heated under reflux for 3 h. The reaction mixture was cooled down to room temperature, solvents were evaporated under reduced pressure to get the crude reaction mixture. It was then diluted with water and washed with EtOAc. Aqueous layer was carefully acidified using 1 (N) HCl to pH=3, extracted with DCM (3×50 mL), dried over Na₂SO₄, filtered, and concentrated in vacuo to afford 6-chloro-3-(3-(naphthalen-1-yloxy)propyl)-7-(1,3,5-trimethyl-1H-pyrazol-4-yl)-1H-indole-2-carboxylic acid (2.3 g, 4.71 mmol, 58%) as brown gummy solid.

LCMS (ESI⁺): m/z 488.4 [M+H]⁺

### Step C

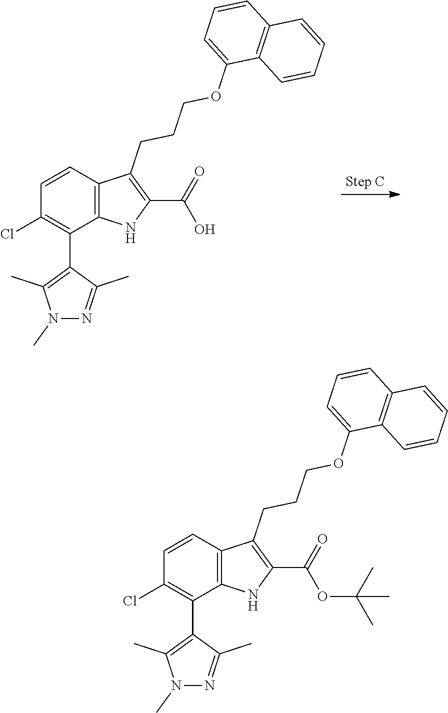 [SMILES: Cc1nn(C)c(C)c1-c1c(Cl)ccc2c(CCCOc3cccc4ccccc34)c(C(=O)O)[nH]c12 ; Cc1nn(C)c(C)c1-c1c(Cl)ccc2c(CCCOc3cccc4ccccc34)c(C(=O)OC(C)(C)C)[nH]c12]

6-Chloro-3-(3-(naphthalen-1-yloxy)propyl)-7-(1,3,5-trimethyl-1H-pyrazol-4-yl)-1H-indole-2-carboxylic acid (2.3 g, 4.71 mmol) was suspended in toluene (50 mL) and the mixture was heated to reflux under nitrogen. N,N-dimethylformamide di-tert-butyl acetal (5.6 mL, 23.4 mmol) was added drop wise to the refluxing mixture. Refluxing was continued for an additional 12 h under nitrogen. After 16 h another 3.4 mL (14.2 mmol) of N,N-dimethylformamide di-tert-butyl acetal was added to it and the reaction was continued for another 12 h. Reaction mixture was then diluted with EtOAc, washed successively with NaHCO₃ (sat), water and brine, organic layer was dried over Na₂SO₄ and evaporated under reduced pressure to get the crude compound, which was then purified by flash chromatography (SiO₂, 50% EtOAc in DCM) to get tert-butyl 6-chloro-3-(3-(naphthalen-1-yloxy)propyl)-7-(1,3,5-trimethyl-1H-pyrazol-4-yl)-1H-indole-2-carboxylate (2.1 g, 3.86 mmol, 82%) as yellow sticky solid.

LCMS (ESI⁺): m/z 544.5 [M+H]⁺

### Step D

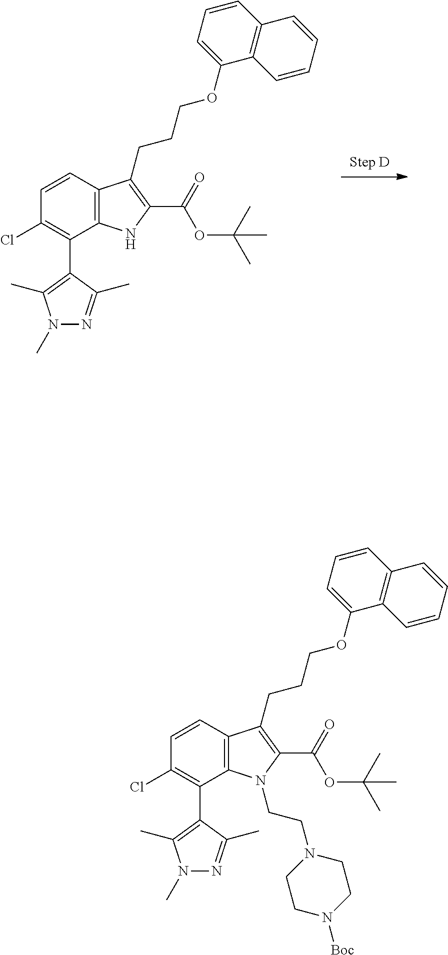 [SMILES: Cc1nn(C)c(C)c1-c1c(Cl)ccc2c(CCCOc3cccc4ccccc34)c(C(=O)OC(C)(C)C)[nH]c12 ; Cc1nn(C)c(C)c1-c1c(Cl)ccc2c(CCCOc3cccc4ccccc34)c(C(=O)OC(C)(C)C)n(CCN3CCN(C(=O)OC(C)(C)C)CC3)c12]

To a well stirred solution of tert-butyl 6-chloro-3-(3-(naphthalen-1-yloxy)propyl)-7-(1,3,5-trimethyl-1H-pyrazol-4-yl)-1H-indole-2-carboxylate (2.1 g, 3.86 mmol) in DMF (20 mL) was added tert-butyl 4-(2-chloroethyl)piperazine-1-carboxylate (1.9 g, 7.6 mmol) followed by Cs₂CO₃ (6.3 g, 19.3 mmol) in DMF and the mixture was allowed to stir at 90° C. for 16 h under nitrogen. The reaction mixture was diluted with EtOAc, washed successively with water and brine, the organic layer was dried over Na₂SO₄ and evaporated under reduced pressure to get the crude compound, which was then purified by flash chromatography (SiO₂, 70% EtOAc in hexane) to get tert-butyl 1-(2-(4-(tert-butoxycarbonyl)piperazin-1-yl)ethyl)-6-chloro-3-(3-(naphthalen-1-yloxy)propyl)-7-(1,3,5-trimethyl-1H-pyrazol-4-yl)-1H-indole-2-carboxylate (2 g, 2.64 mmol, 68%) as off light yellow solid.

LCMS (ESI): m/z 756.2 [M+H]⁺

### Step E

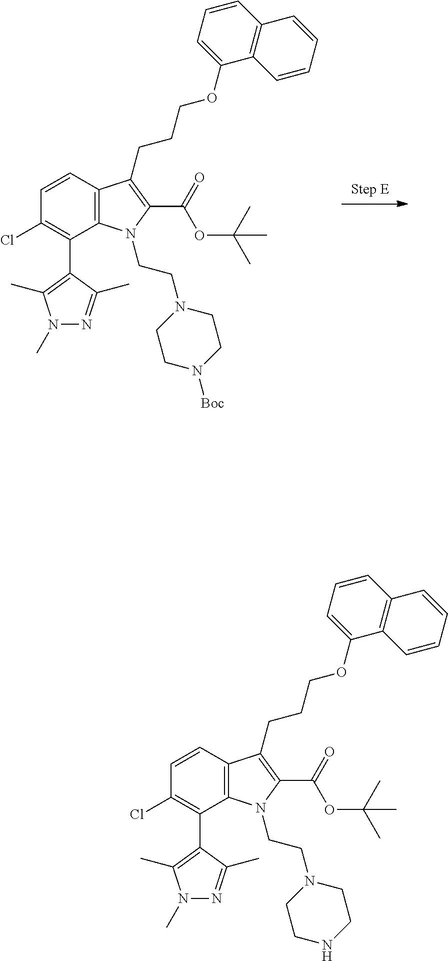 [SMILES: Cc1nn(C)c(C)c1-c1c(Cl)ccc2c(CCCOc3cccc4ccccc34)c(C(=O)OC(C)(C)C)n(CCN3CCN(C(=O)OC(C)(C)C)CC3)c12 ; Cc1nn(C)c(C)c1-c1c(Cl)ccc2c(CCCOc3cccc4ccccc34)c(C(=O)OC(C)(C)C)n(CCN3CCNCC3)c12]

To a stirred solution of tert-butyl 1-(2-(4-(tert-butoxycarbonyl)piperazin-1-yl)ethyl)-6-chloro-3-(3-(naphthalen-1-yloxy)propyl)-7-(1,3,5-trimethyl-1H-pyrazol-4-yl)-1H-indole-2-carboxylate (1.7 g, 2.24 mmol) in dioxane (20 mL) was added 20 mL of 4M HCl in dioxane at 0° C. under nitrogen and the reaction mixture was allowed to stir at room temperature for 1 h. After consuming of starting material (monitored by TLC and LCMS) the reaction mixture was quenched by drop wise addition of aq 1N NaOH at 0° C. and adjusted the pH to 7, then it was extracted with DCM (3×150 mL), dried over Na₂SO₄, filtered, and concentrated in vacuum to afford the crude material which was further purified by triturating by Et₂O and pentane to afford tert-butyl 6-chloro-3-[3-(naphthalen-1-yloxy)propyl]-1-[2-(piperazin-1-yl)ethyl]-7-(1,3,5-trimethyl-1H-pyrazol-4-yl)-1H-indole-2-carboxylate (1 g, 1.52 mmol, 68%) as off white solid.

LCMS (ESI⁺): m/z 656.4 [M+H]⁺

### Step F

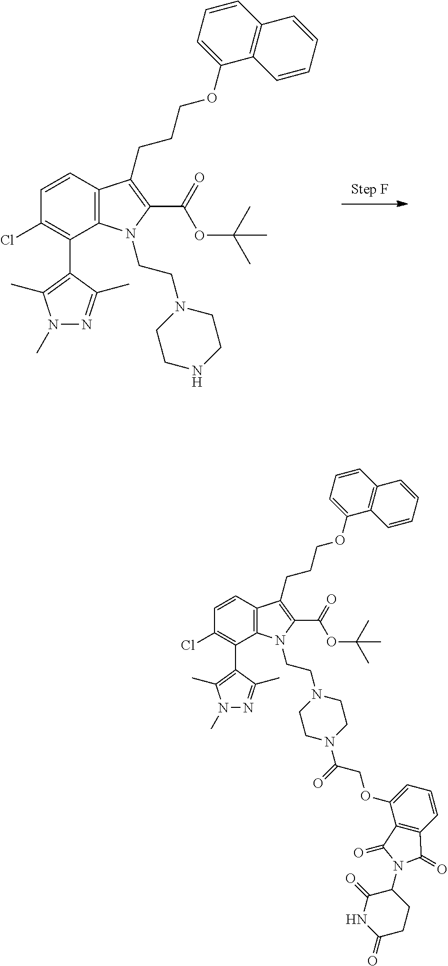 [SMILES: Cc1nn(C)c(C)c1-c1c(Cl)ccc2c(CCCOc3cccc4ccccc34)c(C(=O)OC(C)(C)C)n(CCN3CCNCC3)c12 ; Cc1nn(C)c(C)c1-c1c(Cl)ccc2c(CCCOc3cccc4ccccc34)c(C(=O)OC(C)(C)C)n(CCN3CCN(C(=O)COc4cccc5c4C(=O)N(C4CCC(=O)NC4=O)C5=O)CC3)c12]

To a well stirred solution of 2-((2-(2,6-dioxopiperidin-3-yl)-1,3-dioxoisoindolin-4-yl)oxy)acetic acid (30.4 mg, 0.091 mmol) and tert-butyl 6-chloro-3-[3-(naphthalen-1-yloxy)propyl]-1-[2-(piperazin-1-yl)ethyl]-7-(1,3,5-trimethyl-1H-pyrazol-4-yl)-1H-indole-2-carboxylate (50.0 mg, 0.072 mmol) in DMF (0.762 mL) was added DIPEA (0.040 mL, 0.229 mmol) and HATU (34.8 mg, 0.091 mmol) and the mixture was allowed to stir under nitrogen for 16 h. After complete consumption of the starting material (monitored by TLC and LCMS), then the reaction mixture diluted with EtOAc, washed successively with cold water and brine. Organic layer was dried over Na₂SO₄ and evaporated under reduced pressure. Crude material was purified using flash chromatography (SiO₂, 10% MeOH in DCM) to afford tert-butyl 6-chloro-1-(2-(4-(2-((2-(2,6-dioxopiperidin-3-yl)-1,3-dioxoisoindolin-4-yl)oxy)acetyl)piperazin-1-yl)ethyl)-3-(3-(naphthalen-1-yloxy)propyl)-7-(1,3,5-trimethyl-1H-pyrazol-4-yl)-1H-indole-2-carboxylate (65 mg, 0.067 mmol, 88%) as white solid.

LCMS (ESI⁺): m/z 970.4 [M+H]⁺

### Step G

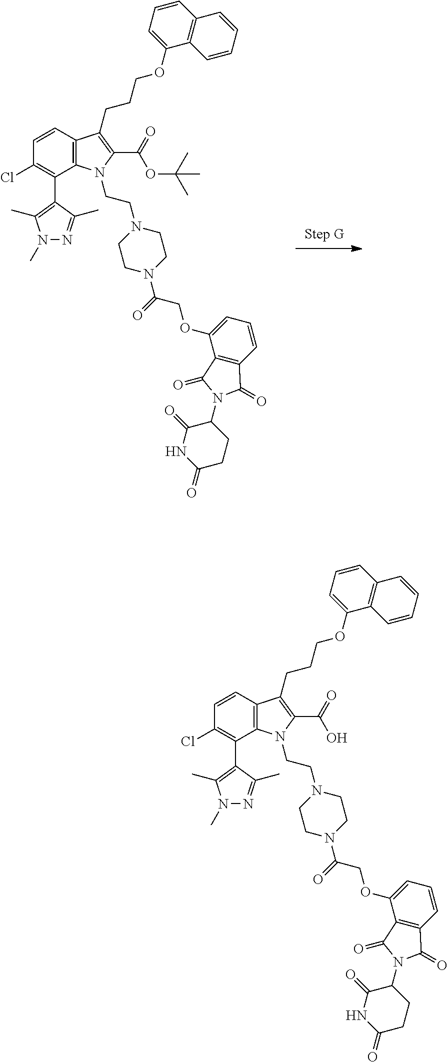 [SMILES: Cc1nn(C)c(C)c1-c1c(Cl)ccc2c(CCCOc3cccc4ccccc34)c(C(=O)OC(C)(C)C)n(CCN3CCN(C(=O)COc4cccc5c4C(=O)N(C4CCC(=O)NC4=O)C5=O)CC3)c12 ; Cc1nn(C)c(C)c1-c1c(Cl)ccc2c(CCCOc3cccc4ccccc34)c(C(=O)O)n(CCN3CCN(C(=O)COc4cccc5c4C(=O)N(C4CCC(=O)NC4=O)C5=O)CC3)c12]

Tert-butyl 6-chloro-1-(2-(4-(2-((2-(2,6-dioxopiperidin-3-yl)-1,3-dioxoisoindolin-4-yl)oxy)acetyl)piperazin-1-yl)ethyl)-3-(3-(naphthalen-1-yloxy)propyl)-7-(1,3,5-trimethyl-1H-pyrazol-4-yl)-1H-indole-2-carboxylate (110.0 mg, 0.11 mmol) was placed in a capped vial, dissolved in DCM and then TFA (0.087 mL, 1.133 mmol) was added. Reaction was stirred overnight at room temperature. Solvents were removed under reduced pressure and crude was purified using preparative HPLC (H₂O:MeCN+0.1% FA) to afford 6-chloro-1-{2-[4-(2-{[2-(2,6-dioxopiperidin-3-yl)-1,3-dioxo-2,3-dihydro-1H-isoindol-4-yl]oxy}acetyl)piperazin-1-yl]ethyl}-3-[3-(naphthalen-1-yloxy)propyl]-7-(1,3,5-trimethyl-1H-pyrazol-4-yl)-1H-indole-2-carboxylic acid (50.0 mg, 0.055 mmol, 49%) as white solid.

LCMS (ESI⁺): m/z 914.4 [M+H]⁺

¹H NMR (500 MHz, DMSO) δ 13.43 (s, 1H), 11.11 (s, 1H), 8.26-8.19 (m, 1H), 7.91-7.85 (m, 1H), 7.81-7.73 (m, 2H), 7.57-7.49 (m, 2H), 7.46 (dd, J=7.7, 6.1 Hz, 2H), 7.40 (t, J=7.9 Hz, 1H), 7.31 (d, J=8.6 Hz, 1H), 7.24 (d, J=8.5 Hz, 1H), 6.92 (dd, J=7.7, 1.0 Hz, 1H), 5.19-5.06 (m, 3H), 4.36-4.27 (m, 1H), 4.27-4.14 (m, 3H), 3.78 (s, 3H), 3.31-3.27 (m, 2H), 2.91 (ddd, J=16.8, 13.8, 5.4 Hz, 1H), 2.65-2.53 (m, 2H), 2.24 (p, J=6.5 Hz, 2H), 2.19-1.99 (m, 10H), 1.90 (s, 3H). 4 protons in aliphatic area overlaps with water.

### Example 4: 6-chloro-1-(2-(4-(2-((2-(1-methyl-2,6-dioxopiperidin-3-yl)-1,3-dioxoisoindolin-4-yl)oxy)acetyl)piperazin-1-yl)ethyl)-3-(3-(naphthalen-1-yloxy)propyl)-7-(1,3,5-trimethyl-1H-pyrazol-4-yl)-1H-indole-2-carboxylic acid (205)

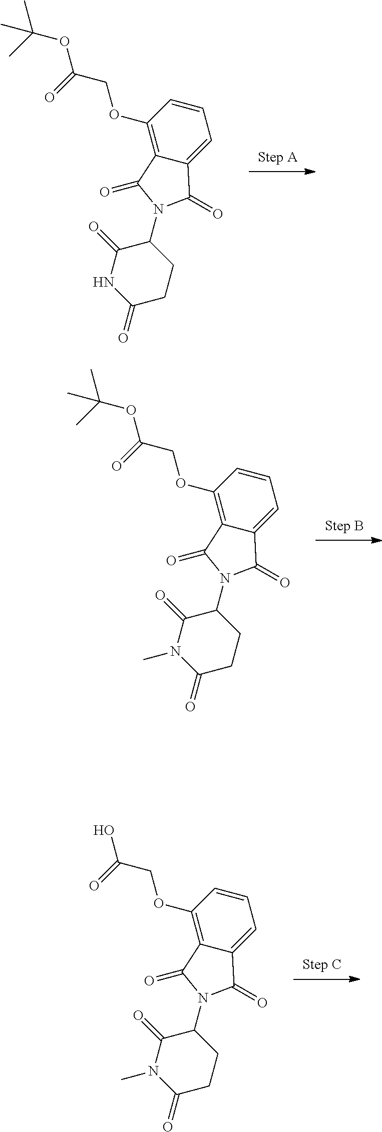

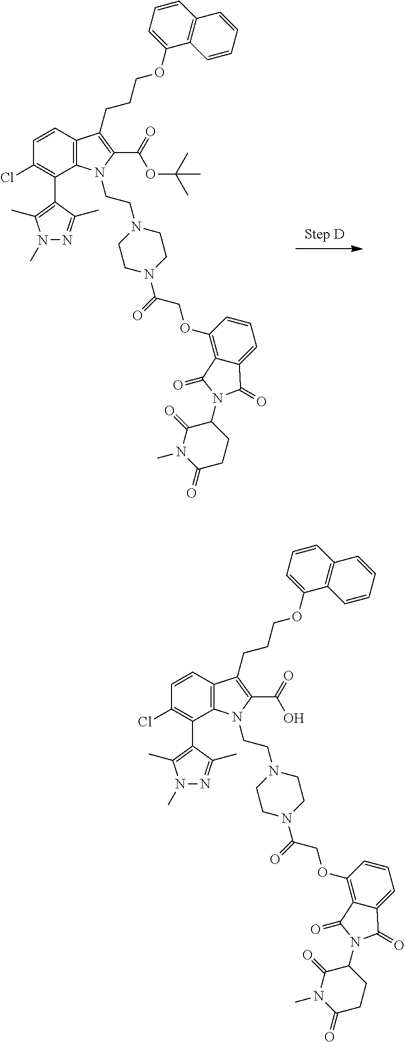 [SMILES: Cc1nn(C)c(C)c1-c1c(Cl)ccc2c(CCCOc3cccc4ccccc34)c(C(=O)OC(C)(C)C)n(CCN3CCN(C(=O)COc4cccc5c4C(=O)N(C4CCC(=O)N(C)C4=O)C5=O)CC3)c12 ; Cc1nn(C)c(C)c1-c1c(Cl)ccc2c(CCCOc3cccc4ccccc34)c(C(=O)O)n(CCN3CCN(C(=O)COc4cccc5c4C(=O)N(C4CCC(=O)N(C)C4=O)C5=O)CC3)c12]

### Step A

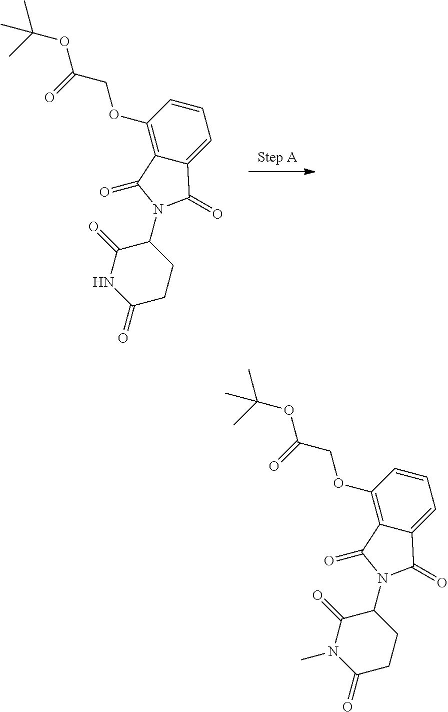 [SMILES: CC(C)(C)OC(=O)COc1cccc2c1C(=O)N(C1CCC(=O)NC1=O)C2=O ; CN1C(=O)CCC(N2C(=O)c3cccc(OCC(=O)OC(C)(C)C)c3C2=O)C1=O]

Tert-butyl 2-{[2-(2,6-dioxopiperidin-3-yl)-1,3-dioxo-2,3-dihydro-1H-isoindol-4-yl]oxy}acetate (50.0 mg, 0.129 mmol) was dissolved in acetone (1.3 mL). Potassium carbonate (53.4 mg, 0.386 mmol) was added and the reaction mixture was cooled down to 0° C. Next, iodomethane was (0.012 mL, 0.193 mmol) was slowly added and the reaction mixture was stirred at room temperature for 16 h. Water was added to the mixture and reaction product was extracted with DCM. Solvents were removed under reduced pressure and reaction product tert-butyl 2-((2-(1-methyl-2,6-dioxopiperidin-3-yl)-1,3-dioxoisoindolin-4-yl)oxy)acetate (22.0 mg, 0.055 mmol, 43%) was used to the next step.

### Step B

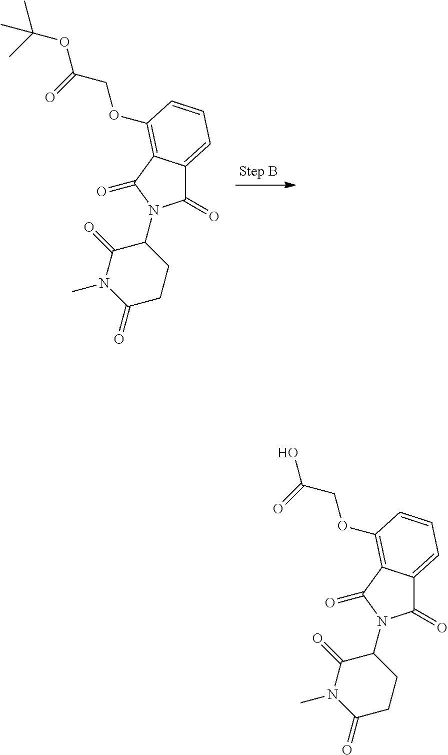 [SMILES: CN1C(=O)CCC(N2C(=O)c3cccc(OCC(=O)OC(C)(C)C)c3C2=O)C1=O ; CN1C(=O)CCC(N2C(=O)c3cccc(OCC(=O)O)c3C2=O)C1=O]

Tert-butyl 2-{[2-(1-methyl-2,6-dioxopiperidin-3-yl)-1,3-dioxo-2,3-dihydro-1H-isoindol-4-yl]oxy}acetate (21.0 mg, 0.052 mmol) was dissolved in DCM (0.522 mL), and trifluoroacetic acid (0.080 mL, 1.044 mmol) was added. The mixture was stirred for 4 h. Reaction mixture was separated between water and DCM. Organic phase was collected, solvent evaporated and the resulting product_(2-{[2-(1-methyl-2,6-dioxopiperidin-3-yl)-1,3-dioxo-2,3-dihydro-1H-isoindol-4-yl]oxy}acetic acid (13.3 mg, 0.038 mmol, 74%) was obtained as white solid and subjected to the subsequent step.

LCMS (ESI+): m/z 347.2 [M+H]⁺

### Step C

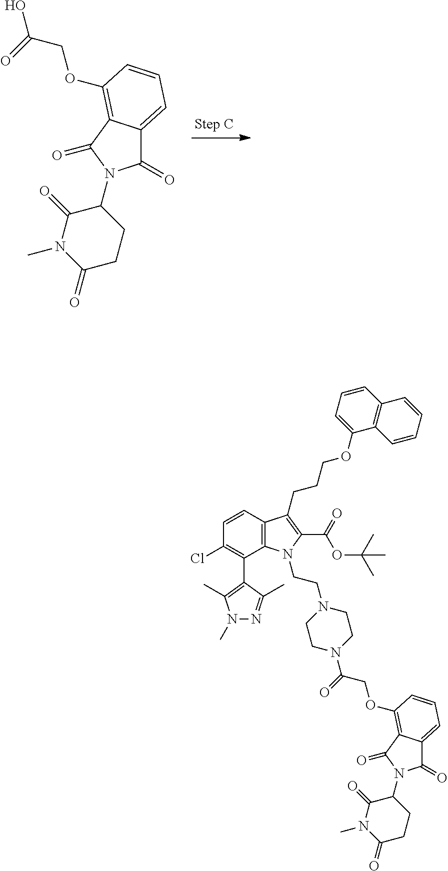 [SMILES: CN1C(=O)CCC(N2C(=O)c3cccc(OCC(=O)O)c3C2=O)C1=O ; Cc1nn(C)c(C)c1-c1c(Cl)ccc2c(CCCOc3cccc4ccccc34)c(C(=O)OC(C)(C)C)n(CCN3CCN(C(=O)COc4cccc5c4C(=O)N(C4CCC(=O)N(C)C4=O)C5=O)CC3)c12]

2-{[2-(1-methyl-2,6-dioxopiperidin-3-yl)-1,3-dioxo-2,3-dihydro-1H-isoindol-4-yl]oxy}acetic acid (13.3 mg, 0.038 mmol) was dissolved in DMF (1.3 mL). DIPEA (0.020 mL, 0.115 mmol) followed by HATU (17.5 mg, 0.046 mmol) were added and the reaction stirred for 15 min in room temperature. Next, tert-butyl 6-chloro-3-(3-(naphthalen-1-yloxy)propyl)-1-(2-(piperazin-1-yl)ethyl)-7-(1,3,5-trimethyl-1H-pyrazol-4-yl)-1H-indole-2-carboxylate (30.2 mg, 0.046 mmol) was added and the reaction stirred for 2 h. After that time DMF was removed, the resulting solid dissolved in EtOAc and washed 3× with water. Organic layer was collected, dried over Na₂SO₄, filtered and concentrated in vacuum. Solvents were evaporated and reaction product dried under reduced pressure to give 32.0 mg of crude tert-butyl 6-chloro-1-(2-(4-(2-((2-(1-methyl-2,6-dioxopiperidin-3-yl)-1,3-dioxoisoindolin-4-yl)oxy)acetyl)piperazin-1-yl)ethyl)-3-(3-(naphthalen-1-yloxy)propyl)-7-(1,3,5-trimethyl-1H-pyrazol-4-yl)-1H-indole-2-carboxylate that was used in the next step without further purification.

LCMS (ESI+): m/z 984.8 [M+H]⁺

### Step D

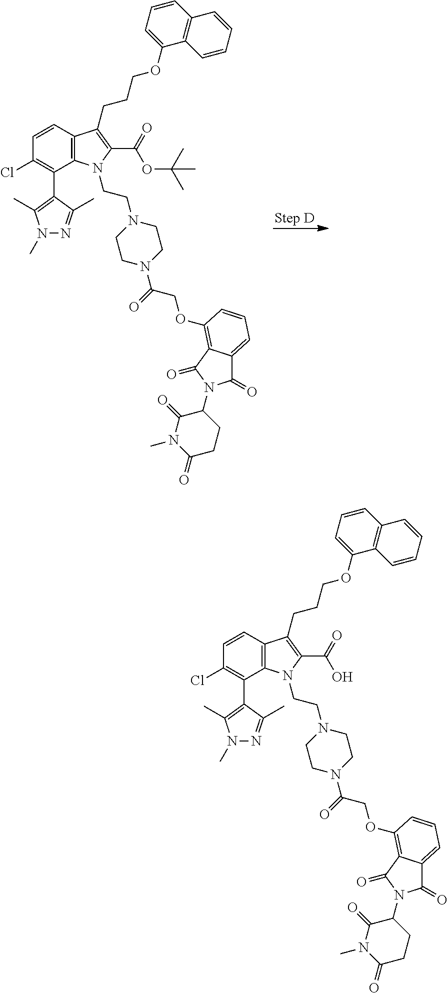 [SMILES: Cc1nn(C)c(C)c1-c1c(Cl)ccc2c(CCCOc3cccc4ccccc34)c(C(=O)OC(C)(C)C)n(CCN3CCN(C(=O)COc4cccc5c4C(=O)N(C4CCC(=O)N(C)C4=O)C5=O)CC3)c12 ; Cc1nn(C)c(C)c1-c1c(Cl)ccc2c(CCCOc3cccc4ccccc34)c(C(=O)O)n(CCN3CCN(C(=O)COc4cccc5c4C(=O)N(C4CCC(=O)N(C)C4=O)C5=O)CC3)c12]

Tert-butyl 6-chloro-1-(2-(4-(2-((2-(1-methyl-2,6-dioxopiperidin-3-yl)-1,3-dioxoisoindolin-4-yl)oxy)acetyl)piperazin-1-yl)ethyl)-3-(3-(naphthalen-1-yloxy)propyl)-7-(1,3,5-trimethyl-1H-pyrazol-4-yl)-1H-indole-2-carboxylate (32.0 mg, crude) was dissolved in dry DCM (0.25 mL) under an inert gas atmosphere. TFA (0.25 mL, 3.250 mmol) was added and the reaction was stirred in room temperature. After 18 h full conversion of the starting material was observed. DCM and TFA acid were evaporated under reduced pressure and the resulting solid dissolved in DMSO and purified by preparative HPLC (H₂O:MeCN+0.1% FA) to give a corresponding 6-chloro-1-(2-(4-(2-((2-(1-methyl-2,6-dioxopiperidin-3-yl)-1,3-dioxoisoindolin-4-yl)oxy)acetyl)piperazin-1-yl)ethyl)-3-(3-(naphthalen-1-yloxy)propyl)-7-(1,3,5-trimethyl-1H-pyrazol-4-yl)-1H-indole-2-carboxylic acid (18.0 mg, 0.019 mmol, 50% over two steps) as white solid.

LCMS (ESI+): m/z 928.8 [M+H]⁺

¹H NMR (500 MHz, DMSO) δ 13.42 (s, 1H), 8.28-8.19 (m, 1H), 7.94-7.84 (m, 1H), 7.83-7.73 (m, 2H), 7.62-7.50 (m, 2H), 7.50-7.44 (m, 2H), 7.41 (t, J=7.9 Hz, 1H), 7.33 (d, J=8.4 Hz, 1H), 7.26 (d, J=8.5 Hz, 1H), 6.93 (d, J=7.5 Hz, 1H), 5.25-5.06 (m, 3H), 4.47-4.11 (m, 4H), 3.78 (s, 3H), 3.47-3.35 (m, 6H), 3.02 (s, 3H), 2.99-2.87 (m, 1H), 2.82-2.74 (m, 1H), 2.62-2.53 (m, 1H), 2.30-1.97 (m, 12H), 1.91 (s, 3H).

### Example 5: 6-chloro-1-(2-(4-(2-((2-(2,6-dioxopiperidin-3-yl)-1-oxoisoindolin-4-yl)oxy)acetyl)piperazin-1-yl)ethyl)-3-(3-(naphthalen-1-yloxy)propyl)-7-(1,3,5-trimethyl-1H-pyrazol-4-yl)-1H-indole-2-carboxylic acid (206)

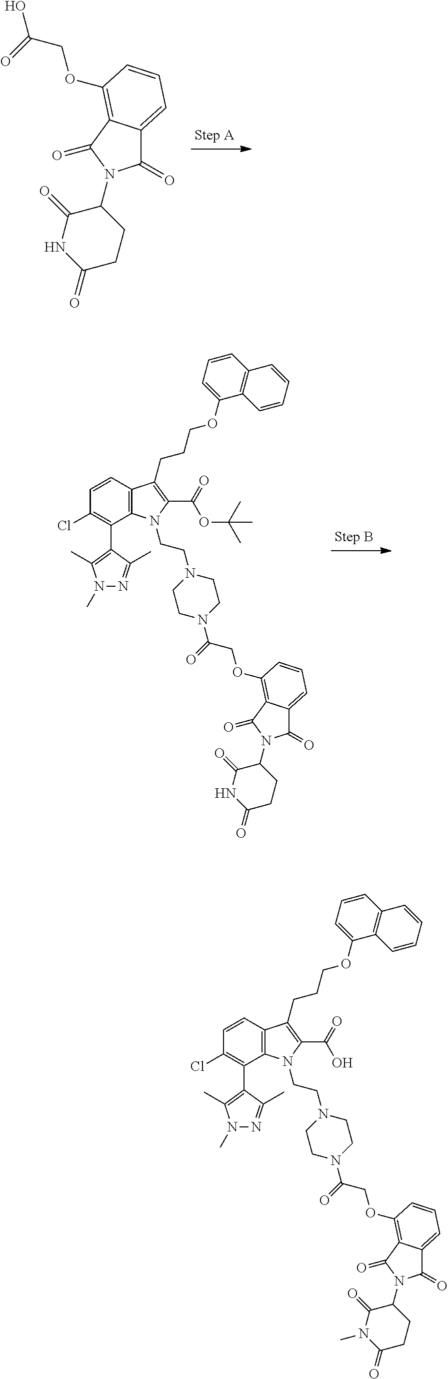 [SMILES: O=C(O)COc1cccc2c1C(=O)N(C1CCC(=O)NC1=O)C2=O ; Cc1nn(C)c(C)c1-c1c(Cl)ccc2c(CCCOc3cccc4ccccc34)c(C(=O)OC(C)(C)C)n(CCN3CCN(C(=O)COc4cccc5c4C(=O)N(C4CCC(=O)NC4=O)C5=O)CC3)c12 ; Cc1nn(C)c(C)c1-c1c(Cl)ccc2c(CCCOc3cccc4ccccc34)c(C(=O)O)n(CCN3CCN(C(=O)COc4cccc5c4C(=O)N(C4CCC(=O)N(C)C4=O)C5=O)CC3)c12]

### Step A

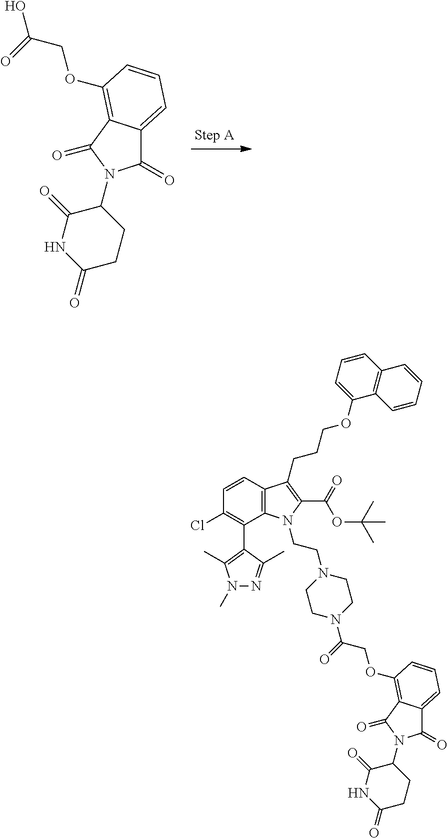 [SMILES: O=C(O)COc1cccc2c1C(=O)N(C1CCC(=O)NC1=O)C2=O ; Cc1nn(C)c(C)c1-c1c(Cl)ccc2c(CCCOc3cccc4ccccc34)c(C(=O)OC(C)(C)C)n(CCN3CCN(C(=O)COc4cccc5c4C(=O)N(C4CCC(=O)NC4=O)C5=O)CC3)c12]

2-((2-(2,6-dioxopiperidin-3-yl)-1-oxoisoindolin-4-yl)oxy)acetic acid (15.0 mg, 0.047 mmol) was dissolved in dry DMF (0.943 mL) under inert gas atmosphere. DIPEA (0.025 mL, 0.141 mmol) followed by HATU (26.9 mg, 0.071 mmol) were added and the reaction mixture stirred for 15 min in room temperature. Tert-butyl 6-chloro-3-(3-(naphthalen-1-yloxy)propyl)-1-(2-(piperazin-1-yl)ethyl)-7-(1,3,5-trimethyl-1H-pyrazol-4-yl)-1H-indole-2-carboxylate (30.9 mg, 0.047 mmol) was added and the solution stirred for additional 2 h. DMF was removed under reduced pressure, crude product dissolved in EtOAc and washed 3× with water. Organic layer was collected and dried over Na₂SO₄. Solvent was evaporated to get crude (45 mg) tert-butyl 6-chloro-1-(2-(4-(2-((2-(2,6-dioxopiperidin-3-yl)-1-oxoisoindolin-4-yl)oxy)acetyl)piperazin-1-yl)ethyl)-3-(3-(naphthalen-1-yloxy)propyl)-7-(1,3,5-trimethyl-1H-pyrazol-4-yl)-1H-indole-2-carboxylate as dark solid.

LCMS (ESI+): 956.8 m/z [M+H]⁺

### Step B

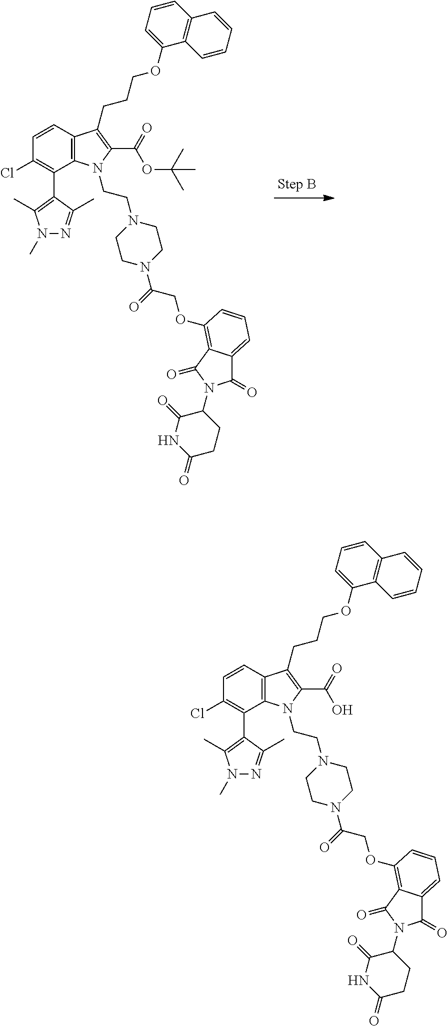 [SMILES: Cc1nn(C)c(C)c1-c1c(Cl)ccc2c(CCCOc3cccc4ccccc34)c(C(=O)OC(C)(C)C)n(CCN3CCN(C(=O)COc4cccc5c4C(=O)N(C4CCC(=O)NC4=O)C5=O)CC3)c12 ; Cc1nn(C)c(C)c1-c1c(Cl)ccc2c(CCCOc3cccc4ccccc34)c(C(=O)O)n(CCN3CCN(C(=O)COc4cccc5c4C(=O)N(C4CCC(=O)NC4=O)C5=O)CC3)c12]

Tert-butyl 6-chloro-1-(2-(4-(2-((2-(2,6-dioxopiperidin-3-yl)-1-oxoisoindolin-4-yl)oxy)acetyl)piperazin-1-yl)ethyl)-3-(3-(naphthalen-1-yloxy)propyl)-7-(1,3,5-trimethyl-1H-pyrazol-4-yl)-1H-indole-2-carboxylate (45.0 mg, crude) was dissolved in dry DCM (0.1 mL). TFA was added (0.100 mL, 1.303 mmol) and the reaction was stirred at room temperature for 16 h. DCM and TFA acid were evaporated under reduced pressure and the resulting solid dissolved in DMSO and was purified by preparative HPLC (H₂O:MeCN+0.1% FA) to give 6-chloro-1-(2-(4-(2-((2-(2,6-dioxopiperidin-3-yl)-1-oxoisoindolin-4-yl)oxy)acetyl)piperazin-1-yl)ethyl)-3-(3-(naphthalen-1-yloxy)propyl)-7-(1,3,5-trimethyl-1H-pyrazol-4-yl)-1H-indole-2-carboxylic acid (16.8 mg, 0.019 mmol, 38% yield over two steps) as white powder.

LCMS (ESI+): m/z 899.9 [M+H]⁺

¹H NMR (500 MHz, DMSO) δ 13.42 (s, 1H), 10.98 (s, 1H), 8.22 (d, J=9.0 Hz, 1H), 7.90-7.84 (m, 1H), 7.76 (d, J=8.2 Hz, 1H), 7.53 (dq, J=6.8, 5.4 Hz, 2H), 7.46 (t, J=8.0 Hz, 2H), 7.40 (t, J=7.9 Hz, 1H), 7.33 (d, J=7.4 Hz, 1H), 7.24 (d, J=8.4 Hz, 1H), 7.13 (d, J=8.2 Hz, 1H), 6.92 (d, J=7.5 Hz, 1H), 5.12 (dd, J=13.3, 5.1 Hz, 1H), 4.96 (s, 2H), 4.42-4.14 (m, 6H), 3.77 (d, J=1.7 Hz, 3H), 3.41-3.34 (m, 6H), 3.01-2.85 (m, 1H), 2.63-2.57 (m, 2H), 2.29-2.18 (m, 2H), 2.15-1.99 (m, 10H), 1.89 (s, 3H).

### Example 6: 6-chloro-1-{2-[4-(2-{[2-(2,6-dioxopiperidin-3-yl)-1,3-dioxo-2,3-dihydro-1H-isoindol-4-yl]amino}acetyl)piperazin-1-yl]ethyl}-3-[3-(naphthalen-1-yloxy)propyl]-7-(1,3,5-trimethyl-1H-pyrazol-4-yl)-1H-indole-2-carboxylic acid) (207)

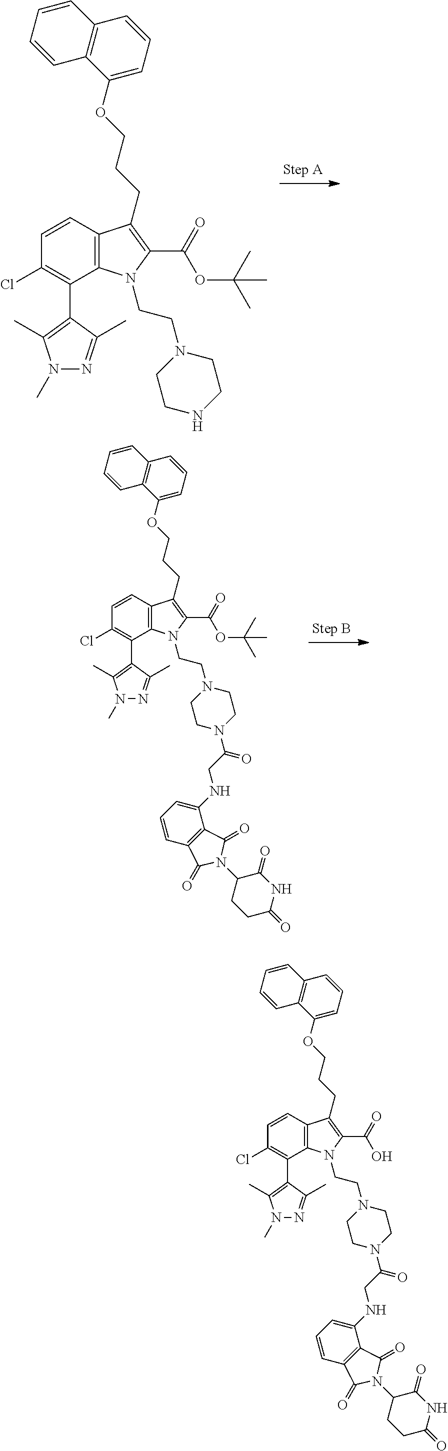 [SMILES: Cc1nn(C)c(C)c1-c1c(Cl)ccc2c(CCCOc3cccc4ccccc34)c(C(=O)OC(C)(C)C)n(CCN3CCNCC3)c12 ; Cc1nn(C)c(C)c1-c1c(Cl)ccc2c(CCCOc3cccc4ccccc34)c(C(=O)OC(C)(C)C)n(CCN3CCN(C(=O)CNc4cccc5c4C(=O)N(C4CCC(=O)NC4=O)C5=O)CC3)c12 ; Cc1nn(C)c(C)c1-c1c(Cl)ccc2c(CCCOc3cccc4ccccc34)c(C(=O)O)n(CCN3CCN(C(=O)CNc4cccc5c4C(=O)N(C4CCC(=O)NC4=O)C5=O)CC3)c12]

### Step A

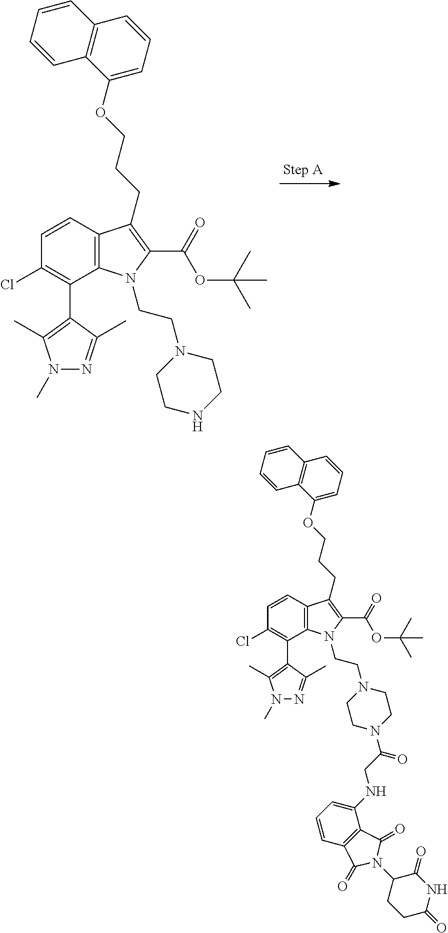 [SMILES: Cc1nn(C)c(C)c1-c1c(Cl)ccc2c(CCCOc3cccc4ccccc34)c(C(=O)OC(C)(C)C)n(CCN3CCNCC3)c12 ; Cc1nn(C)c(C)c1-c1c(Cl)ccc2c(CCCOc3cccc4ccccc34)c(C(=O)OC(C)(C)C)n(CCN3CCN(C(=O)CNc4cccc5c4C(=O)N(C4CCC(=O)NC4=O)C5=O)CC3)c12]

To a well stirred solution of 2-{[2-(2,6-dioxopiperidin-3-yl)-1,3-dioxo-2,3-dihydro-1H-isoindol-4-yl]amino}acetic acid (30.3 mg, 0.091 mmol) and tert-butyl 6-chloro-3-(3-(naphthalen-1-yloxy)propyl)-1-(2-(piperazin-1-yl)ethyl)-7-(1,3,5-trimethyl-1H-pyrazol-4-yl)-1H-indole-2-carboxylate (50.0 mg, 0.076 mmol) in DMF (0.762 mL) was added DIPEA (0.040 mL, 0.229 mmol) and HATU (43.5 mg, 0.114 mmol) and the mixture was allowed to stir under nitrogen for 2 h at room temperature. The reaction mixture diluted with EtOAc, washed successively with cold water and brine. Organic layer was dried over Na₂SO₄ and evaporated under reduced pressure. The crude tert-butyl 6-chloro-1-{2-[4-(2-{[2-(2,6-dioxopiperidin-3-yl)-1,3-dioxo-2,3-dihydro-1H-isoindol-4-yl]amino}acetyl)piperazin-1-yl]ethyl}-3-[3-(naphthalen-1-yloxy)propyl]-7-(1,3,5-trimethyl-1H-pyrazol-4-yl)-1H-indole-2-carboxylate (68.7 mg) was obtained as yellow gum, which was used for the next step without further purification.

LCMS (ESI+): m/z 969.3 [M+H]⁺

### Step B

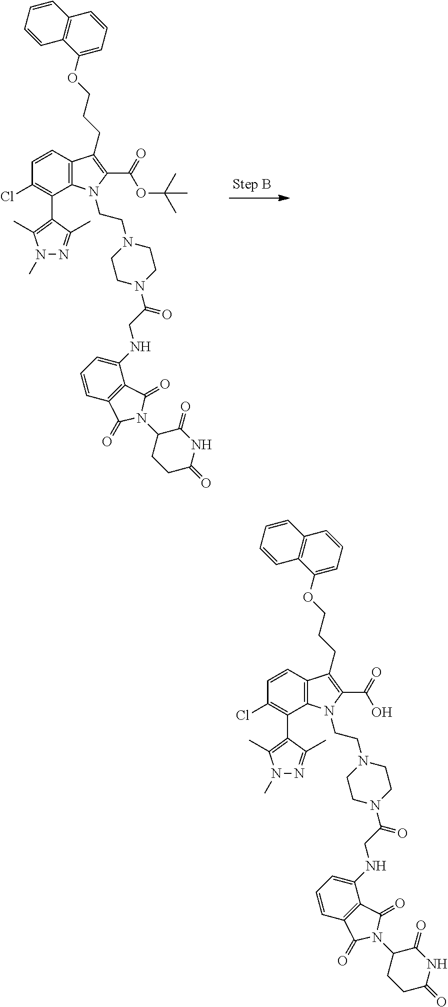 [SMILES: Cc1nn(C)c(C)c1-c1c(Cl)ccc2c(CCCOc3cccc4ccccc34)c(C(=O)OC(C)(C)C)n(CCN3CCN(C(=O)CNc4cccc5c4C(=O)N(C4CCC(=O)NC4=O)C5=O)CC3)c12 ; Cc1nn(C)c(C)c1-c1c(Cl)ccc2c(CCCOc3cccc4ccccc34)c(C(=O)O)n(CCN3CCN(C(=O)CNc4cccc5c4C(=O)N(C4CCC(=O)NC4=O)C5=O)CC3)c12]

Tert-butyl 6-chloro-1-{2-[4-(2-{[2-(2,6-dioxopiperidin-3-yl)-1,3-dioxo-2,3-dihydro-1H-isoindol-4-yl]amino}acetyl)piperazin-1-yl]ethyl}-3-[3-(naphthalen-1-yloxy)propyl]-7-(1,3,5-trimethyl-1H-pyrazol-4-yl)-1H-indole-2-carboxylate (68.7 mg, crude) was dissolved in DCM (0.15 mL) and then TFA (0.054 mL, 0.708 mmol) was added. Reaction was stirred overnight at room temperature. Solvents were removed under reduced pressure, and crude was purified using preparative HPLC (H₂O:MeCN+0.1% FA) to afford 6-chloro-1-{2-[4-(2-{[2-(2,6-dioxopiperidin-3-yl)-1,3-dioxo-2,3-dihydro-1H-isoindol-4-yl]amino}acetyl)piperazin-1-yl]ethyl}-3-[3-(naphthalen-1-yloxy)propyl]-7-(1,3,5-trimethyl-1H-pyrazol-4-yl)-1H-indole-2-carboxylic acid (21.8 mg, 0.024 mmol, 32% over two steps) as yellow solid.

LCMS (ESI+): m/z 913.2 [M+H]⁺

¹H NMR (500 MHz, DMSO) δ 13.40 (s, 1H), 11.10 (s, 1H), 8.25-8.19 (m, 1H), 7.90-7.84 (m, 1H), 7.77 (d, J=8.6 Hz, 1H), 7.66-7.56 (m, 2H), 7.56-7.50 (m, 2H), 7.47 (d, J=8.3 Hz, 1H), 7.40 (t, J=7.9 Hz, 1H), 7.25 (d, J=8.5 Hz, 1H), 7.10-7.08 (m, 1H), 7.07-7.04 (m, 1H), 6.92 (dd, J=7.7, 1.0 Hz, 1H), 5.08 (dd, J=12.8, 5.4 Hz, 1H), 4.35-4.27 (m, 1H), 4.27-4.15 (m, 3H), 4.12 (d, J=4.6 Hz, 2H), 3.79 (s, 3H), 3.48-3.40 (m, 2H), 3.40-3.35 (m, 2H), 3.30-3.25 (m, 2H), 2.90 (ddd, J=16.9, 13.8, 5.4 Hz, 1H), 2.64-2.55 (m, 2H), 2.28-2.20 (m, 2H), 2.16-2.08 (m, 5H), 2.08-2.00 (m, 5H), 1.90 (s, 3H).

### Example 7: 6-chloro-1-(2-((3aR,6aS)-5-(2-((2-(2,6-dioxopiperidin-3-yl)-1,3-dioxoisoindolin-4-yl)oxy)acetyl)hexahydropyrrolo[3,4-c]pyrrol-2(1H)-yl)ethyl)-3-(3-(naphthalen-1-yloxy)propyl)-7-(1,3,5-trimethyl-1H-pyrazol-4-yl)-1H-indole-2-carboxylic acid (211)

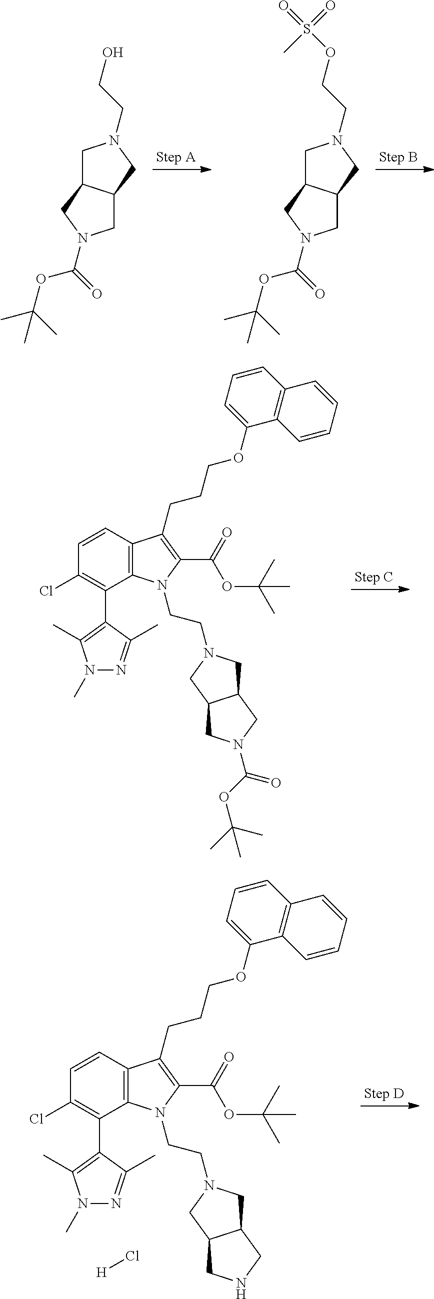

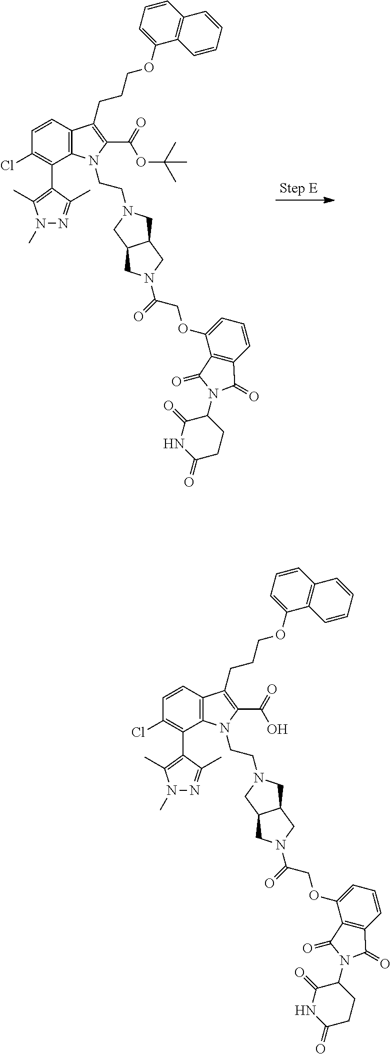 [SMILES: Cc1nn(C)c(C)c1-c1c(Cl)ccc2c(CCCOc3cccc4ccccc34)c(C(=O)OC(C)(C)C)n(CCN3C[C@@H]4CN(C(=O)COc5cccc6c5C(=O)N(C5CCC(=O)NC5=O)C6=O)C[C@@H]4C3)c12 ; Cc1nn(C)c(C)c1-c1c(Cl)ccc2c(CCCOc3cccc4ccccc34)c(C(=O)O)n(CCN3C[C@@H]4CN(C(=O)COc5cccc6c5C(=O)N(C5CCC(=O)NC5=O)C6=O)C[C@@H]4C3)c12]

### Step A

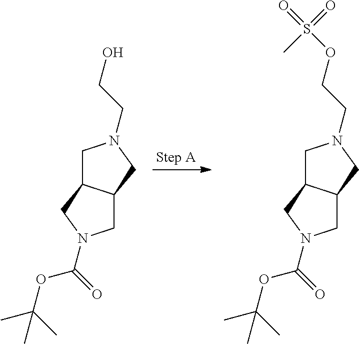 [SMILES: CC(C)(C)OC(=O)N1C[C@H]2CN(CCO)C[C@H]2C1 ; CC(C)(C)OC(=O)N1C[C@H]2CN(CCOS(C)(=O)=O)C[C@H]2C1]

Tert-butyl 5-(2-hydroxyethyl)-octahydropyrrolo[3,4-c]pyrrole-2-carboxylate (485.0 mg, 1.892 mmol) was dissolved in DCM (5.0 mL), Et₃N (0.395 mL, 2.838 mmol) and DMAP (23.1 mg, 0.189 mmol) were added and reaction mixture cooled to 0° C. Then MsCl (0.176 mL, 2.270 mmol) was added dropwise and reaction mixture was let to stir at RT for 4 h and next 36 h in a fridge (at 4° C.). The crude was extracted with brine, dried over Na₂SO₄, filtered and concentrated in vacuo. The product, tert-butyl 5-[2-(methanesulfonyloxy)ethyl]-octahydropyrrolo[3,4-c]pyrrole-2-carboxylate (520.8 mg, 1.557 mmol, 82.3%) was an orange oil.

LCMS (ESI⁺): m/z 334.8 [M+H]⁺

### Step B

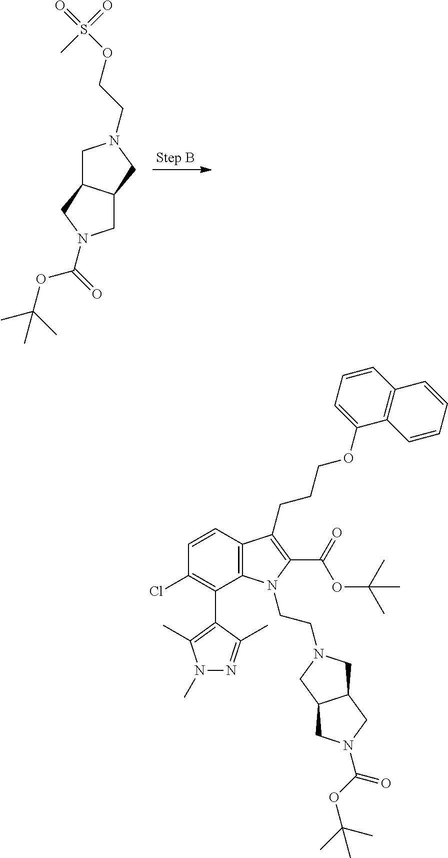 [SMILES: CC(C)(C)OC(=O)N1C[C@H]2CN(CCOS(C)(=O)=O)C[C@H]2C1 ; Cc1nn(C)c(C)c1-c1c(Cl)ccc2c(CCCOc3cccc4ccccc34)c(C(=O)OC(C)(C)C)n(CCN3C[C@@H]4CN(C(=O)OC(C)(C)C)C[C@@H]4C3)c12]

Tert-butyl 6-chloro-3-[3-(naphthalen-1-yloxy)propyl]-7-(1,3,5-trimethyl-1H-pyrazol-4-yl)-1H-indole-2-carboxylate (50.0 mg, 0.092 mmol), tert-butyl 5-[2-(methanesulfonyloxy)ethyl]-octahydropyrrolo[3,4-c]pyrrole-2-carboxylate_(36.9 mg, 0.110 mmol) and Cs₂CO₃ (89.8 mg, 0.276 mmol) were dissolved in dry DMF (2.0 mL) and stirred at 60° C. for overnight. After complete consumption of the starting material solvent was evaporated under reduced pressure, the residues were dissolved in DCM and washed with H₂O and brine. The organic layer was dried over Na₂SO₄, filtered and concentrated in vacuum. Product tert-butyl 1-(2-{5-[(tert-butoxy)carbonyl]-octahydropyrrolo[3,4-c]pyrrol-2-yl}ethyl)-6-chloro-3-[3-(naphthalen-1-yloxy)propyl]-7-(1,3,5-trimethyl-1H-pyrazol-4-yl)-1H-indole-2-carboxylate (71.7 mg, 0.092 mmol) was used to next step without further purification.

LCMS (ESI⁺): m/z 782.0 [M+H]⁺

### Step C

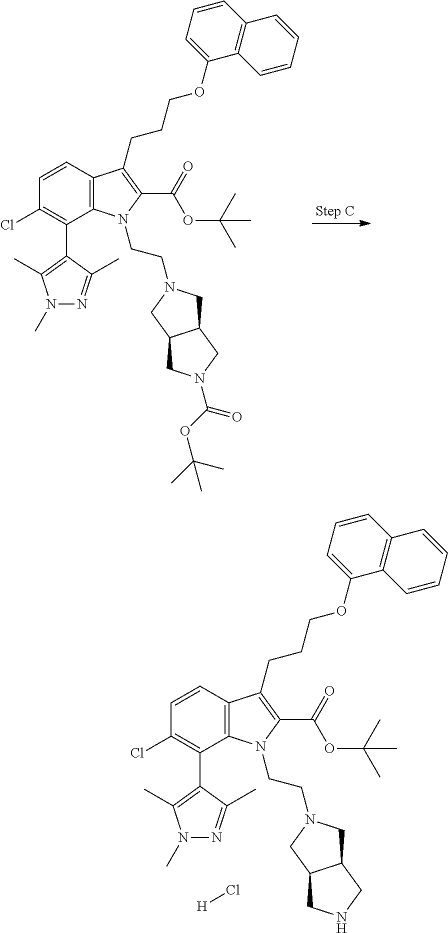 [SMILES: Cc1nn(C)c(C)c1-c1c(Cl)ccc2c(CCCOc3cccc4ccccc34)c(C(=O)OC(C)(C)C)n(CCN3C[C@@H]4CN(C(=O)OC(C)(C)C)C[C@@H]4C3)c12 ; Cc1nn(C)c(C)c1-c1c(Cl)ccc2c(CCCOc3cccc4ccccc34)c(C(=O)OC(C)(C)C)n(CCN3C[C@H]4CNC[C@H]4C3)c12 ; Cl]

To a solution of tert-butyl 1-(2-{5-[(tert-butoxy)carbonyl]-octahydropyrrolo[3,4-c]pyrrol-2-yl}ethyl)-6-chloro-3-[3-(naphthalen-1-yloxy)propyl]-7-(1,3,5-trimethyl-1H-pyrazol-4-yl)-1H-indole-2-carboxylate (71.7 mg, 0.092 mmol) in THE (3.5 mL) at 0° C. was added 4M HCl in dioxane (0.573 mL, 2.291 mmol). The mixture was stirred for next 24 h at RT. LCMS analysis after 24 h confirmed a presence of starting material, therefore next portion of 4M HCl in dioxane was added and a reaction was stirred next 18 h at RT. After complete consumption of the substrate the crude was concentrated under vacuum and a product tert-butyl 6-chloro-3-[3-(naphthalen-1-yloxy)propyl]-1-(2-{octahydropyrrolo[3,4-c]pyrrol-2-yl}ethyl)-7-(1,3,5-trimethyl-1H-pyrazol-4-yl)-1H-indole-2-carboxylate hydrochloride (77.0 mg) was used to next step without further purification.

LCMS (ESI+): m/z 682.0 [M+H]⁺

### Step D

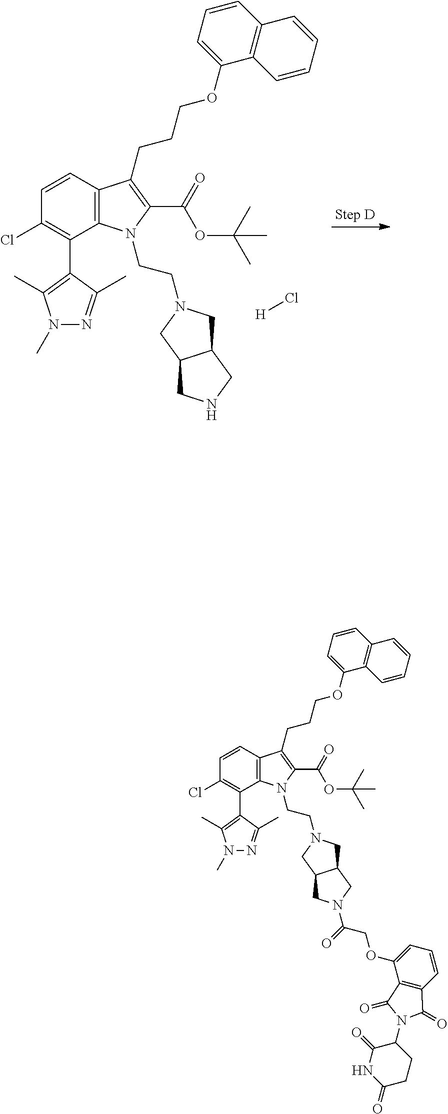 [SMILES: Cc1nn(C)c(C)c1-c1c(Cl)ccc2c(CCCOc3cccc4ccccc34)c(C(=O)OC(C)(C)C)n(CCN3C[C@H]4CNC[C@H]4C3)c12 ; Cl ; Cc1nn(C)c(C)c1-c1c(Cl)ccc2c(CCCOc3cccc4ccccc34)c(C(=O)OC(C)(C)C)n(CCN3C[C@@H]4CN(C(=O)COc5cccc6c5C(=O)N(C5CCC(=O)NC5=O)C6=O)C[C@@H]4C3)c12]

To a well stirred solution of tert-butyl 6-chloro-3-[3-(naphthalen-1-yloxy)propyl]-1-(2-{octahydropyrrolo[3,4-c]pyrrol-2-yl}ethyl)-7-(1,3,5-trimethyl-1H-pyrazol-4-yl)-1H-indole-2-carboxylate hydrochloride (50.0 mg, crude) and 2-{[2-(2,6-dioxopiperidin-3-yl)-1,3-dioxo-2,3-dihydro-1H-isoindol-4-yl]oxy}acetic acid (27.7 mg, 0.083 mmol) in DMF (2.0 mL) were added DIPEA (0.095 mL, 0.545 mmol) and HATU (52.9 mg, 0.139 mmol) and the mixture was allowed to stir under argon for 2 h at RT. After complete consumption of the starting material (monitored by LCMS) reaction mixture was diluted with DCM, washed successively with water and brine. Organic layer was dried over Na₂SO₄ and evaporated under reduced pressure. Product tert-butyl 1-{2-[(3aR,6aS)-5-(2-{[2-(2,6-dioxopiperidin-3-yl)-1,3-dioxo-2,3-dihydro-1H-isoindol-4-yl]oxy}acetyl)-octahydropyrrolo[3,4-c]pyrrol-2-yl]ethyl}-6-chloro-3-[3-(naphthalen-1-yloxy)propyl]-7-(1,3,5-trimethyl-1H-pyrazol-4-yl)-1H-indole-2-carboxylate (87.2 mg, crude) was obtained as brown oil, which was used for the next step without further purification.

LCMS (ESI+): m/z 995.7 [M+H]⁺

### Step E

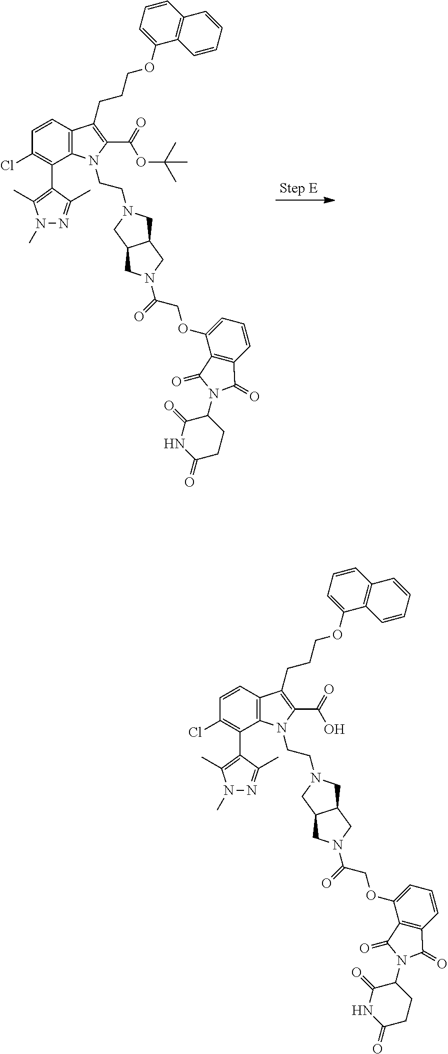 [SMILES: Cc1nn(C)c(C)c1-c1c(Cl)ccc2c(CCCOc3cccc4ccccc34)c(C(=O)OC(C)(C)C)n(CCN3C[C@@H]4CN(C(=O)COc5cccc6c5C(=O)N(C5CCC(=O)NC5=O)C6=O)C[C@@H]4C3)c12 ; Cc1nn(C)c(C)c1-c1c(Cl)ccc2c(CCCOc3cccc4ccccc34)c(C(=O)O)n(CCN3C[C@@H]4CN(C(=O)COc5cccc6c5C(=O)N(C5CCC(=O)NC5=O)C6=O)C[C@@H]4C3)c12]

To a solution of tert-butyl 1-{2-[(3aR,6aS)-5-(2-{[2-(2,6-dioxopiperidin-3-yl)-1,3-dioxo-2,3-dihydro-1H-isoindol-4-yl]oxy}acetyl)-octahydropyrrolo[3,4-c]pyrrol-2-yl]ethyl}-6-chloro-3-[3-(naphthalen-1-yloxy)propyl]-7-(1,3,5-trimethyl-1H-pyrazol-4-yl)-1H-indole-2-carboxylate (87.2 mg crude) in DCM (1.0 mL) was added TFA (1.0 mL, 13.059 mmol). The mixture was stirred for 18 h at RT. After complete consumption of the starting material (monitored by LCMS), the crude was concentrated undervacuum. The residues were dissolved in DMSO and purified by preparative HPLC (H₂O:MeCN+0.1% FA). The isolated product 1-{2-[(3aR,6aS)-5-(2-{[2-(2,6-dioxopiperidin-3-yl)-1,3-dioxo-2,3-dihydro-1H-isoindol-4-yl]oxy}acetyl)-octahydropyrrolo[3,4-c]pyrrol-2-yl]ethyl}-6-chloro-3-[3-(naphthalen-1-yloxy)propyl]-7-(1,3,5-trimethyl-1H-pyrazol-4-yl)-1H-indole-2-carboxylic acid (10.3 mg, 0.011 mmol, 12.0% over four steps) was a white solid.

LCMS (ESI+): m/z 939.8 [M+H]⁺

¹H NMR (500 MHz, DMSO) δ 13.67 (s, 1H), 11.09 (s, 1H), 8.18 (d, J=8.1 Hz, 1H), 7.88-7.82 (m, 1H), 7.79-7.73 (m, 1H), 7.69 (d, J=8.6 Hz, 1H), 7.54-7.45 (m, 2H), 7.45-7.35 (m, 4H), 7.19 (d, J=8.4 Hz, 1H), 6.87 (d, J=7.6 Hz, 1H), 5.09 (dd, J=12.8, 5.4 Hz, 1H), 5.04 (d, J=5.8 Hz, 2H), 4.35-4.23 (m, 1H), 4.23-4.11 (m, 3H), 3.74 (d, J=2.6 Hz, 3H), 3.67 (q, J=9.0 Hz, 1H), 3.57-3.48 (m, 1H), 3.27-3.20 (m, 1H), 3.19-3.10 (m, 1H), 2.89 (ddd, J=17.5, 13.6, 5.4 Hz, 1H), 2.84-2.76 (m, 1H), 2.71-2.52 (m, 4H), 2.35-2.10 (m, 8H), 2.09-1.94 (m, 5H), 1.88 (d, J=4.5 Hz, 3H).

### Example 8: 6-chloro-1-(2-(6-(2-((2-(2,6-dioxopiperidin-3-yl)-1,3-dioxoisoindolin-4-yl)oxy)acetyl)-3,6-diazabicyclo[3.1.1]heptan-3-yl)ethyl)-3-(3-(naphthalen-1-yloxy)propyl)-7-(1,3,5-trimethyl-1H-pyrazol-4-yl)-1H-indole-2-carboxylic acid (208)

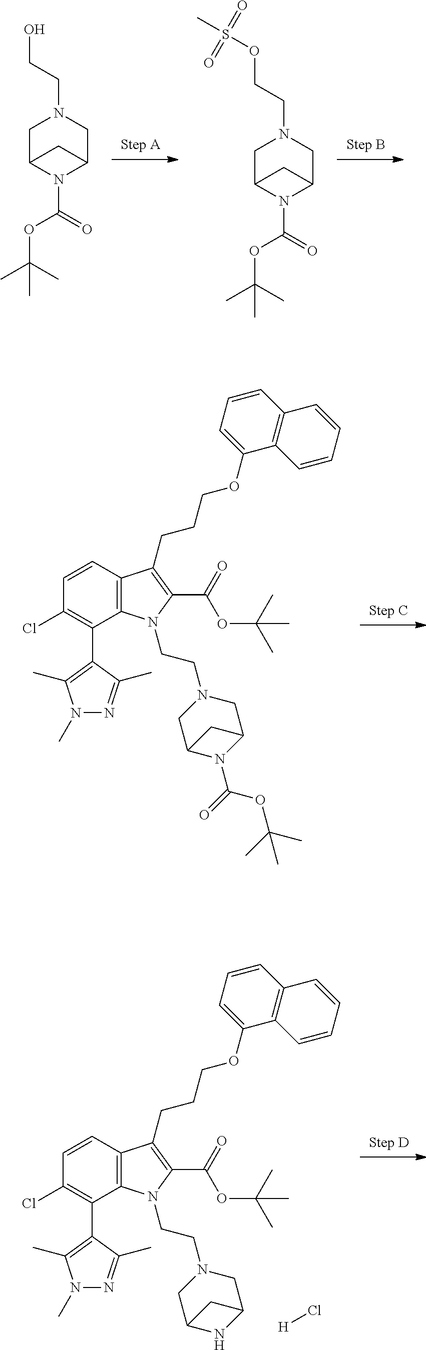

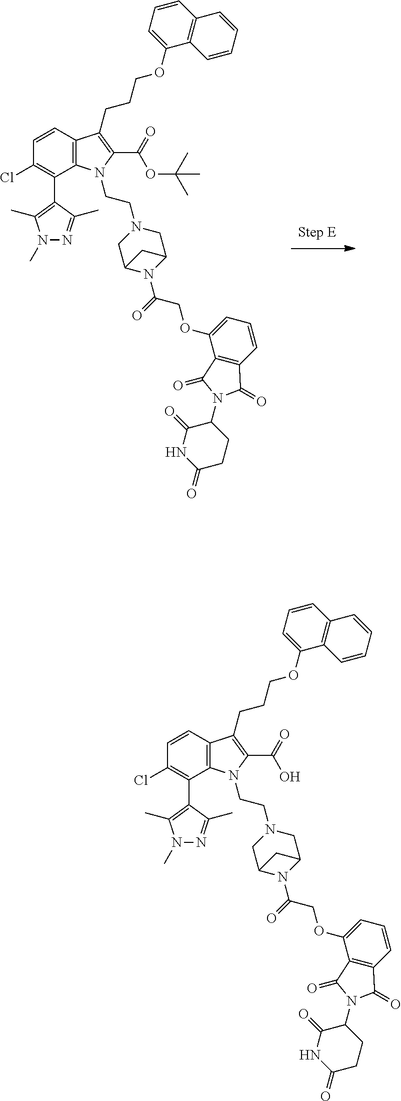 [SMILES: Cc1nn(C)c(C)c1-c1c(Cl)ccc2c(CCCOc3cccc4ccccc34)c(C(=O)OC(C)(C)C)n(CCN3CC4CC(C3)N4C(=O)COc3cccc4c3C(=O)N(C3CCC(=O)NC3=O)C4=O)c12 ; Cc1nn(C)c(C)c1-c1c(Cl)ccc2c(CCCOc3cccc4ccccc34)c(C(=O)O)n(CCN3CC4CC(C3)N4C(=O)COc3cccc4c3C(=O)N(C3CCC(=O)NC3=O)C4=O)c12]

### Step A

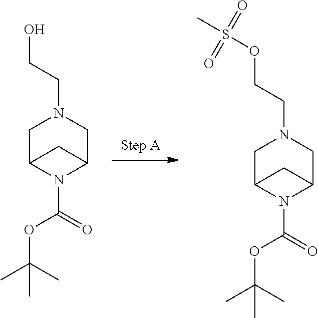 [SMILES: CC(C)(C)OC(=O)N1C2CC1CN(CCO)C2 ; CC(C)(C)OC(=O)N1C2CC1CN(CCOS(C)(=O)=O)C2]

Tert-butyl 3-(2-hydroxyethyl)-3,6-diazabicyclo[3.1.1]heptane-6-carboxylate (132.0 mg, 0.545 mmol) was dissolved in DCM (5.4 mL), Et₃N (0.114 mL, 0.817 mmol) and DMAP (6.7 mg, 0.054 mmol) were added and reaction mixture cooled to −15° C. Then MsCl (0.051 mL, 0.654 mmol) was added dropwise and reaction mixture was let to stir at RT and monitored by TLC (5% MeOH in DCM). Completion of the reaction and formation of one spot was observed after 2 hours. The reaction mixture was diluted in EtOAc and washed with brine. The organic layer was dried over MgSO₄, filtered and concentrated in vacuo to get 35.0 mg of crude tert-butyl 3-[2-(methanesulfonyloxy)ethyl]-3,6-diazabicyclo[3.1.1]heptane-6-carboxylate, which was used directly in the next step without further purification.

### Step B

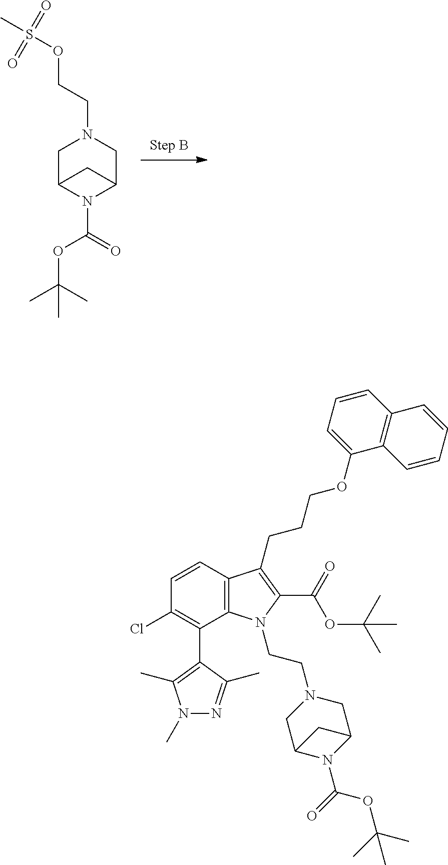 [SMILES: CC(C)(C)OC(=O)N1C2CC1CN(CCOS(C)(=O)=O)C2 ; Cc1nn(C)c(C)c1-c1c(Cl)ccc2c(CCCOc3cccc4ccccc34)c(C(=O)OC(C)(C)C)n(CCN3CC4CC(C3)N4C(=O)OC(C)(C)C)c12]

Tert-butyl 6-chloro-3-[3-(naphthalen-1-yloxy)propyl]-7-(1,3,5-trimethyl-1H-pyrazol-4-yl)-1H-indole-2-carboxylate (50.0 mg, 0.092 mmol), tert-butyl 3-[2-(methanesulfonyloxy)ethyl]-3,6-diazabicyclo[3.1.1]heptane-6-carboxylate (35.0 mg) and Cs₂CO₃ (89.8 mg, 0.276 mmol) were placed in a vial, dissolved in dry DMF (1.8 mL) and stirred at 60° C. for overnight. Another portion of tert-butyl 3-[2-(methanesulfonyloxy)ethyl]-3,6-diazabicyclo[3.1.1]heptane-6-carboxylate (30.0 mg, 0.093 mmol) was added and reaction mixture was stirred for additional 18 hours at 60° C. The reaction mixture was diluted in EtOAc and washed with brine. The organic layer was dried over MgSO₄, filtered and concentrated in vacuo to get 64.0 mg of crude tert-butyl 1-(2-{6-[(tert-butoxy)carbonyl]-3,6-diazabicyclo[3.1.1]heptan-3-yl}ethyl)-6-chloro-3-[3-(naphthalen-1-yloxy)propyl]-7-(1,3,5-trimethyl-1H-pyrazol-4-yl)-1H-indole-2-carboxylate, which was used directly to the next step.

LCMS (ESI⁺): m/z 768.3 [M+H]⁺

### Step C

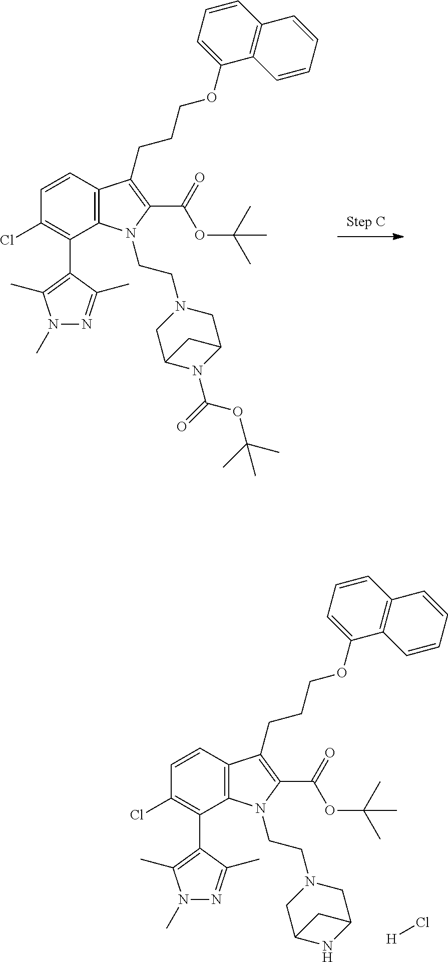

Tert-butyl 1-(2-{6-[(tert-butoxy)carbonyl]-3,6-diazabicyclo[3.1.1]heptan-3-yl}ethyl)-6-chloro-3-[3-(naphthalen-1-yloxy)propyl]-7-(1,3,5-trimethyl-1H-pyrazol-4-yl)-1H-indole-2-carboxylate (64.0 mg) was dissolved in THE (1.7 mL) and reaction mixture was cooled to 0° C. 4M HCl in dioxane (0.521 mL, 2.082 mmol) was then added dropwise and reaction was let to warm up to RT and stirred overnight. After complete consumption of the substrate (monitored by LCMS), solvent was evaporated and crude of tert-butyl 6-chloro-1-(2-{3,6-diazabicyclo[3.1.1]heptan-3-yl}ethyl)-3-[3-(naphthalen-1-yloxy)propyl]-7-(1,3,5-trimethyl-1H-pyrazol-4-yl)-1H-indole-2-carboxylate hydrochloride was used directly to the next step.

LCMS (ESI⁺): m/z 668.3 [M+H]⁺

### Step D

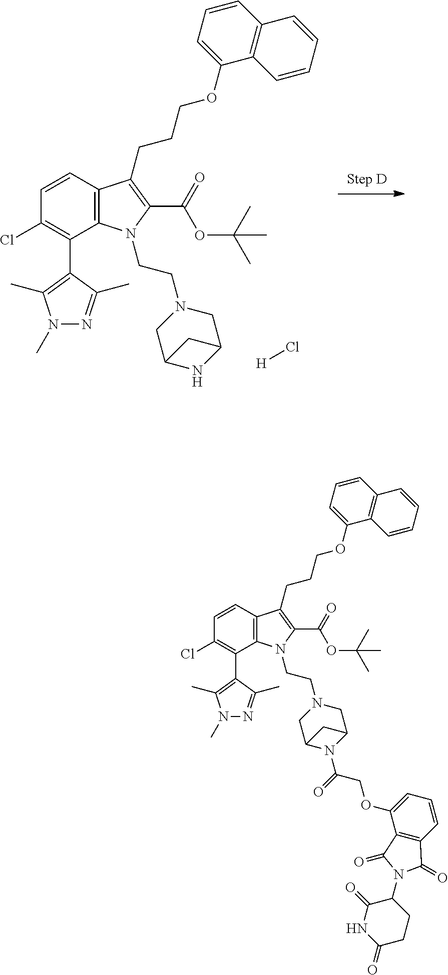 [SMILES: Cc1nn(C)c(C)c1-c1c(Cl)ccc2c(CCCOc3cccc4ccccc34)c(C(=O)OC(C)(C)C)n(CCN3CC4CC(C3)N4)c12 ; Cl ; Cc1nn(C)c(C)c1-c1c(Cl)ccc2c(CCCOc3cccc4ccccc34)c(C(=O)OC(C)(C)C)n(CCN3CC4CC(C3)N4C(=O)COc3cccc4c3C(=O)N(C3CCC(=O)NC3=O)C4=O)c12]

Tert-butyl 6-chloro-1-(2-{3,6-diazabicyclo[3.1.1]heptan-3-yl}ethyl)-3-[3-(naphthalen-1-yloxy)propyl]-7-(1,3,5-trimethyl-1H-pyrazol-4-yl)-1H-indole-2-carboxylate hydrochloride (10.0 mg, 0.014 mmol), 2-{[2-(2,6-dioxopiperidin-3-yl)-1,3-dioxo-2,3-dihydro-1H-isoindol-4-yl]oxy}acetic acid (5.7 mg, 0.017 mmol) and HATU (8.1 mg, 0.021 mmol) were placed in a vial, dissolved in dry DMF (0.167 mL) and then DIPEA (0.010 mL, 0.057 mmol) was added. Reaction was stirred at RT overnight. After complete consumption of the substrate (monitored by LCMS), solvent was evaporated and 56.0 mg crude mixture of tert-butyl 6-chloro-1-{2-[6-(2-{[2-(2,6-dioxopiperidin-3-yl)-1,3-dioxo-2,3-dihydro-1H-isoindol-4-yl]oxy}acetyl)-3,6-diazabicyclo[3.1.1]heptan-3-yl]ethyl}-3-[3-(naphthalen-1-yloxy)propyl]-7-(1,3,5-trimethyl-1H-pyrazol-4-yl)-1H-indole-2-carboxylate was used directly to the next step.

LCMS (ESI⁺): m/z 982.3 [M+H]⁺

### Step E

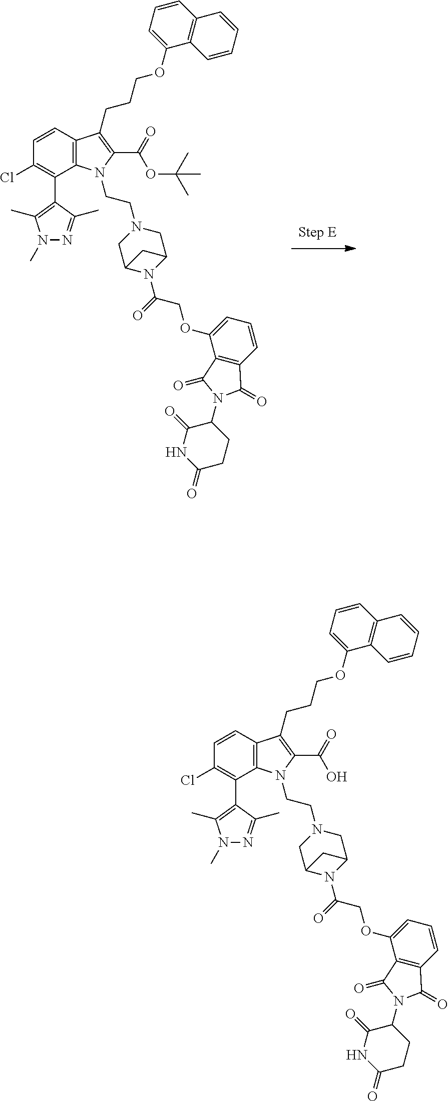 [SMILES: Cc1nn(C)c(C)c1-c1c(Cl)ccc2c(CCCOc3cccc4ccccc34)c(C(=O)OC(C)(C)C)n(CCN3CC4CC(C3)N4C(=O)COc3cccc4c3C(=O)N(C3CCC(=O)NC3=O)C4=O)c12 ; Cc1nn(C)c(C)c1-c1c(Cl)ccc2c(CCCOc3cccc4ccccc34)c(C(=O)O)n(CCN3CC4CC(C3)N4C(=O)COc3cccc4c3C(=O)N(C3CCC(=O)NC3=O)C4=O)c12]

56 mg crude mixture of tert-butyl 6-chloro-1-{2-[6-(2-{[2-(2,6-dioxopiperidin-3-yl)-1,3-dioxo-2,3-dihydro-1H-isoindol-4-yl]oxy}acetyl)-3,6-diazabicyclo[3.1.1]heptan-3-yl]ethyl}-3-[3-(naphthalen-1-yloxy)propyl]-7-(1,3,5-trimethyl-1H-pyrazol-4-yl)-1H-indole-2-carboxylate was dissolved in DCM (1.1 mL), TFA (0.109 mL, 1.425 mmol) was added and reaction was stirred at RT overnight. Another portion of TFA was added (0.500 mL, 6.537 mmol) and reaction was stirred at RT overnight. Solvent was evaporated and crude mixture purified by preparative TLC (20% MeOH in DCM) and repurified by preparative HPLC (H₂O:MeCN+0.1% FA) to get 6-chloro-1-{2-[6-(2-{[2-(2,6-dioxopiperidin-3-yl)-1,3-dioxo-2,3-dihydro-1H-isoindol-4-yl]oxy}acetyl)-3,6-diazabicyclo[3.1.1]heptan-3-yl]ethyl}-3-[3-(naphthalen-1-yloxy)propyl]-7-(1,3,5-trimethyl-1H-pyrazol-4-yl)-1H-indole-2-carboxylic acid (2.6 mg, 0.003 mmol, 21% over two steps) as a white solid.

LCMS (ESI+): m/z 926.0 [M+H]⁺

¹H NMR (500 MHz, DMSO) δ 10.77 (s, 1H), 8.24-8.18 (m, 1H), 7.90-7.82 (m, 1H), 7.82-7.77 (m, 1H), 7.72 (d, J=8.6 Hz, 1H), 7.53-7.46 (m, 3H), 7.43 (t, J=9.2 Hz, 2H), 7.38 (t, J=7.8 Hz, 1H), 7.21 (d, J=8.6 Hz, 1H), 6.90 (d, J=7.7 Hz, 1H), 5.05 (dd, J=12.5, 5.5 Hz, 1H), 4.80 (s, 2H), 4.32-4.22 (m, 4H), 3.73 (s, 3H), 3.30 (t, J=7.6 Hz, 2H), 2.93-2.89 (m, 2H), 2.90-2.82 (m, 1H), 2.82-2.75 (m, 3H), 2.62-2.57 (m, 3H), 2.44-2.39 (m, 2H), 2.30-2.19 (m, 2H), 2.13-1.95 (m, 6H), 1.88 (s, 3H).

### Example 9: 6-chloro-1-(2-(7-(2-((2-(2,6-dioxopiperidin-3-yl)-1,3-dioxoisoindolin-4-yl)oxy)acetyl)-4,7-diazaspiro[2.5]octan-4-yl)ethyl)-3-(3-(naphthalen-1-yloxy)propyl)-7-(1,3,5-trimethyl-1H-pyrazol-4-yl)-1H-indole-2-carboxylic acid (210)

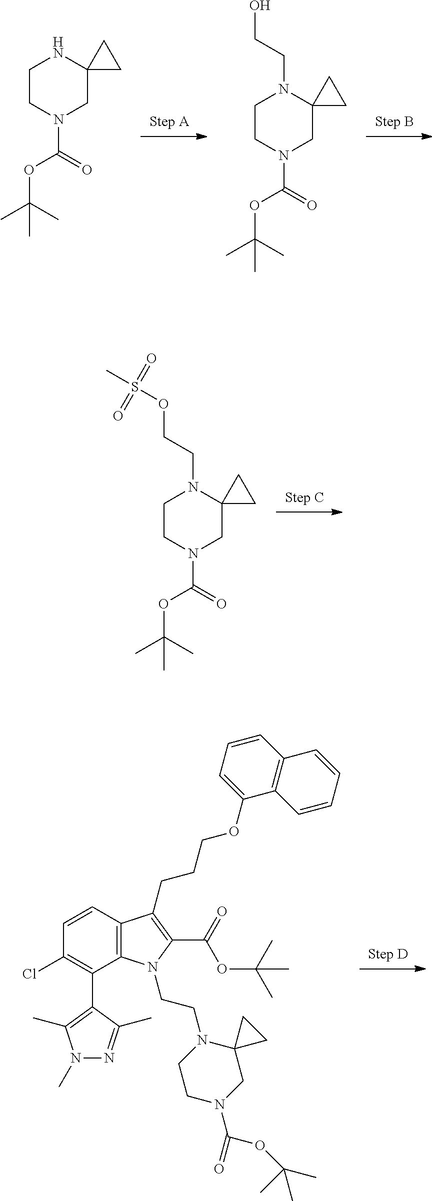 [SMILES: CC(C)(C)OC(=O)N1CCNC2(CC2)C1 ; CC(C)(C)OC(=O)N1CCN(CCO)C2(CC2)C1 ; CC(C)(C)OC(=O)N1CCN(CCOS(C)(=O)=O)C2(CC2)C1 ; Cc1nn(C)c(C)c1-c1c(Cl)ccc2c(CCCOc3cccc4ccccc34)c(C(=O)OC(C)(C)C)n(CCN3CCN(C(=O)OC(C)(C)C)CC34CC4)c12]

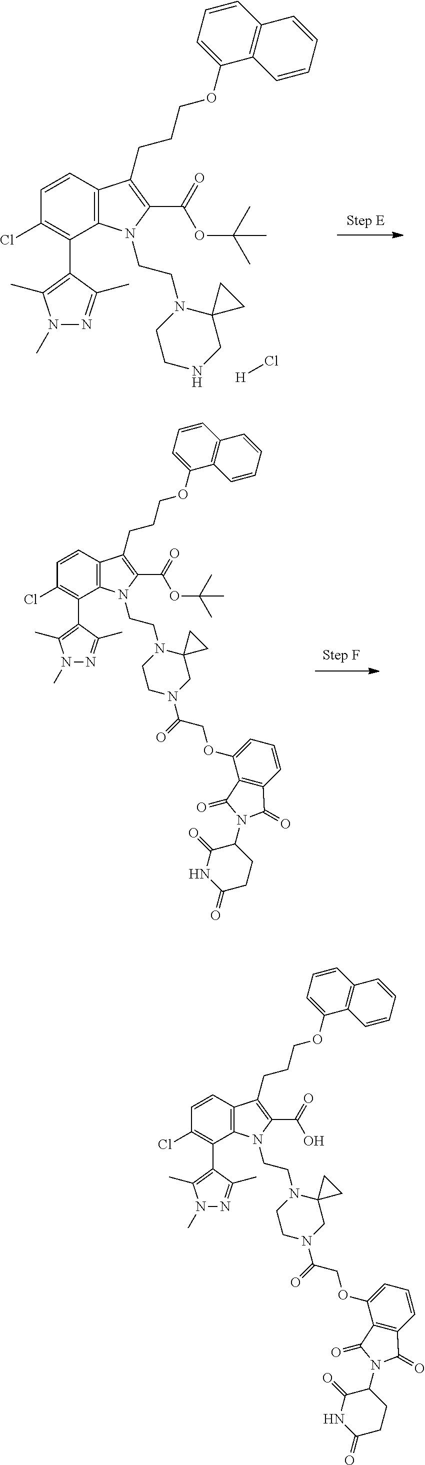 [SMILES: Cc1nn(C)c(C)c1-c1c(Cl)ccc2c(CCCOc3cccc4ccccc34)c(C(=O)OC(C)(C)C)n(CCN3CCNCC34CC4)c12 ; Cl ; Cc1nn(C)c(C)c1-c1c(Cl)ccc2c(CCCOc3cccc4ccccc34)c(C(=O)OC(C)(C)C)n(CCN3CCN(C(=O)COc4cccc5c4C(=O)N(C4CCC(=O)NC4=O)C5=O)CC34CC4)c12 ; Cc1nn(C)c(C)c1-c1c(Cl)ccc2c(CCCOc3cccc4ccccc34)c(C(=O)O)n(CCN3CCN(C(=O)COc4cccc5c4C(=O)N(C4CCC(=O)NC4=O)C5=O)CC34CC4)c12]

### Step A

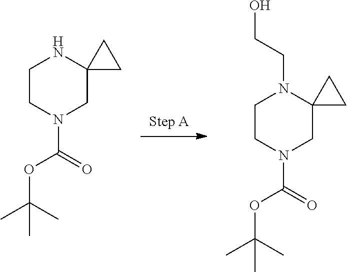 [SMILES: CC(C)(C)OC(=O)N1CCNC2(CC2)C1 ; CC(C)(C)OC(=O)N1CCN(CCO)C2(CC2)C1]

Tert-butyl 4,7-diazaspiro[2.5]octane-7-carboxylate (100.0 mg, 0.471 mmol) and K₂CO₃ (195.3 mg, 1.413 mmol) were placed in a flask, dissolved in dry DMF (5.5 mL) and then 2-bromoethanol (0.167 mL, 2.355 mmol) was added. Reaction was stirred at 80° C. and monitored by TLC (10% MeOH/DCM, R_{f}=0.6). After 18 hours full conversion was observed and formation of one spot (presumably product). The reaction mixture was diluted in EtOAc and washed with brine. The organic layer was dried over magnesium sulfate, filtered and concentrated to dryness. Tert-butyl 4-(2-hydroxyethyl)-4,7-diazaspiro[2.5]octane-7-carboxylate (115 mg, crude) was used directly in the next step without further purification.

### Step B

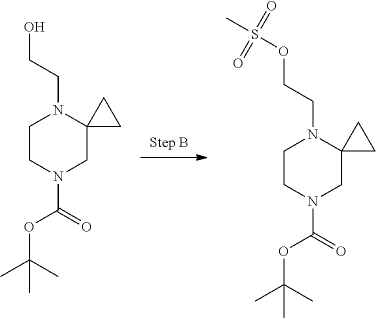 [SMILES: CC(C)(C)OC(=O)N1CCN(CCO)C2(CC2)C1 ; CC(C)(C)OC(=O)N1CCN(CCOS(C)(=O)=O)C2(CC2)C1]

Tert-butyl 4-(2-hydroxyethyl)-4,7-diazaspiro[2.5]octane-7-carboxylate (115.0 mg, crude) was dissolved in DCM (4.5 mL), Et₃N (0.094 mL, 0.673 mmol) and DMAP (5.5 mg, 0.045 mmol) were added and reaction mixture cooled to −15° C. Then MsCl (0.042 mL, 0.538 mmol) was added dropwise and reaction mixture was let to stir at RT. After 30 minutes reaction mixture was diluted in EtOAc and washed with brine. The organic layer was dried over magnesium sulfate, filtered and concentrated to dryness. Tert-butyl 4-(2-((methylsulfonyl)oxy)ethyl)-4,7-diazaspiro[2.5]octane-7-carboxylate (143.8 mg, crude) was used directly in the next step without further purification.

### Step C

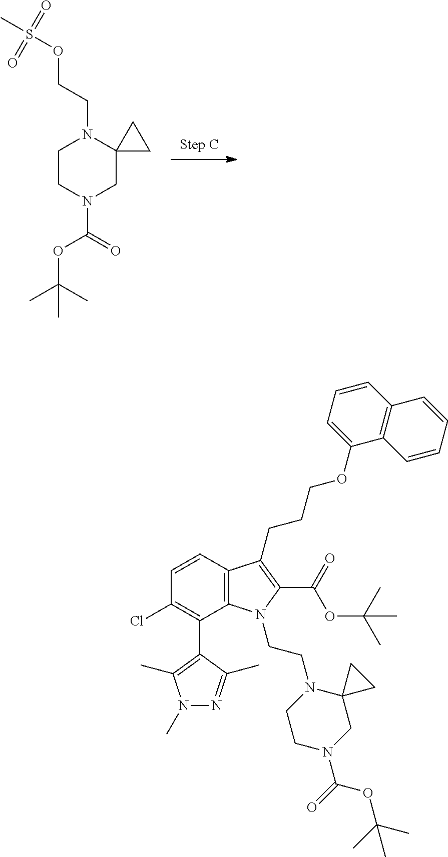 [SMILES: CC(C)(C)OC(=O)N1CCN(CCOS(C)(=O)=O)C2(CC2)C1 ; Cc1nn(C)c(C)c1-c1c(Cl)ccc2c(CCCOc3cccc4ccccc34)c(C(=O)OC(C)(C)C)n(CCN3CCN(C(=O)OC(C)(C)C)CC34CC4)c12]

Tert-butyl 6-chloro-3-[3-(naphthalen-1-yloxy)propyl]-7-(1,3,5-trimethyl-1H-pyrazol-4-yl)-1H-indole-2-carboxylate (195.0 mg, 0.358 mmol), tert-butyl 4-[2-(methanesulfonyloxy)ethyl]-4,7-diazaspiro[2.5]octane-7-carboxylate (143.8 mg, crude) and Cs₂CO₃ (350.3 mg, 1.075 mmol) were placed in a vial, dissolved in dry DMF (7.2 mL) and stirred at 60° C. overnight. The reaction mixture was diluted with EtOAc and washed with brine. The organic layer was dried over magnesium sulfate, filtered and concentrated to dryness. The crude mixture of tert-butyl 1-(2-(7-(tert-butoxycarbonyl)-4,7-diazaspiro[2.5]octan-4-yl)ethyl)-6-chloro-3-(3-(naphthalen-1-yloxy)propyl)-7-(1,3,5-trimethyl-1H-pyrazol-4-yl)-1H-indole-2-carboxylate (276.0 mg, crude) was used directly in the next step.

LCMS (ESI⁺): m/z 782.2 [M+H]⁺

### Step D

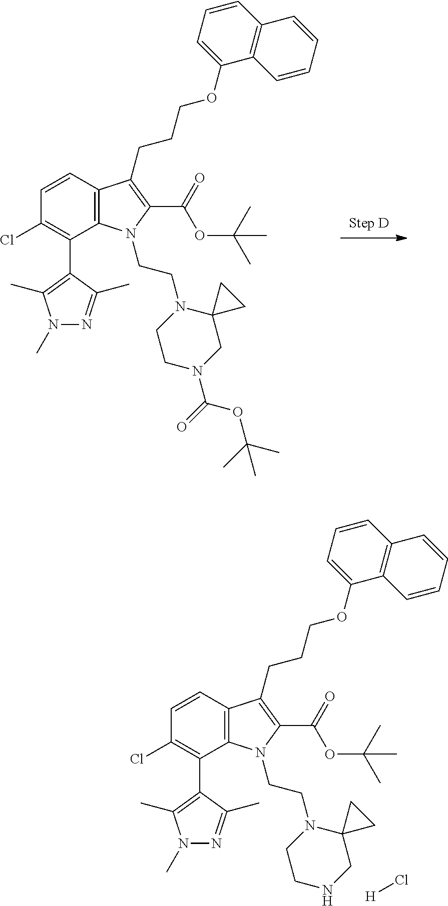

Tert-butyl 1-(2-{7-[(tert-butoxy)carbonyl]-4,7-diazaspiro[2.5]octan-4-yl}ethyl)-6-chloro-3-[3-(naphthalen-1-yloxy)propyl]-7-(1,3,5-trimethyl-1H-pyrazol-4-yl)-1H-indole-2-carboxylate (276.0 mg, crude) was dissolved in THE (3.6 mL), cooled to −15° C. and then 4 M HCl in dioxane (1.1 mL, 4.498 mmol) was added dropwise. Reaction was let to stir at RT overnight. Solvent was evaporated, and crude mixture of tert-butyl 6-chloro-1-(2-{4,7-diazaspiro[2.5]octan-4-yl}ethyl)-3-[3-(naphthalen-1-yloxy)propyl]-7-(1,3,5-trimethyl-1H-pyrazol-4-yl)-1H-indole-2-carboxylate hydrochloride (290.0 mg, crude) was used directly in the next step.

LCMS (ESI⁺): m/z 682.3 [M+H]⁺

### Step E

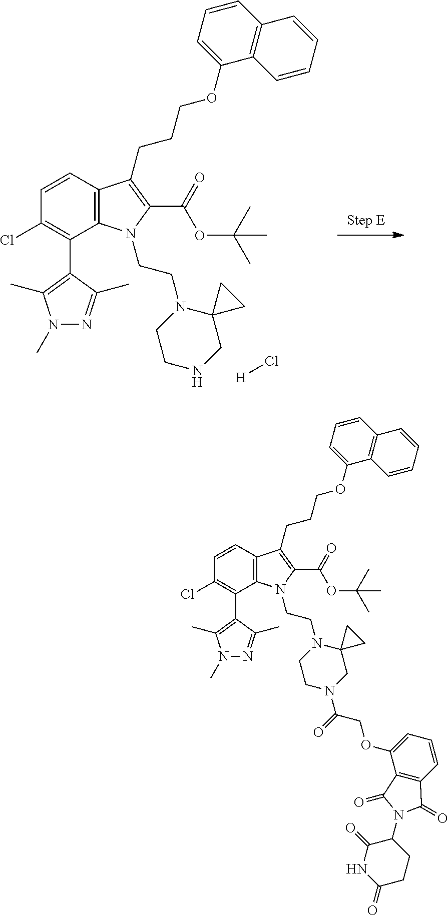 [SMILES: Cc1nn(C)c(C)c1-c1c(Cl)ccc2c(CCCOc3cccc4ccccc34)c(C(=O)OC(C)(C)C)n(CCN3CCNCC34CC4)c12 ; Cl ; Cc1nn(C)c(C)c1-c1c(Cl)ccc2c(CCCOc3cccc4ccccc34)c(C(=O)OC(C)(C)C)n(CCN3CCN(C(=O)COc4cccc5c4C(=O)N(C4CCC(=O)NC4=O)C5=O)CC34CC4)c12]

Tert-butyl 6-chloro-1-(2-{4,7-diazaspiro[2.5]octan-4-yl}ethyl)-3-[3-(naphthalen-1-yloxy)propyl]-7-(1,3,5-trimethyl-1H-pyrazol-4-yl)-1H-indole-2-carboxylate hydrochloride (290.0 mg, crude), 2-{[2-(2,6-dioxopiperidin-3-yl)-1,3-dioxo-2,3-dihydro-1H-isoindol-4-yl]oxy}acetic acid (40.2 mg, 0.121 mmol) and HATU (52.2 mg, 0.137 mmol) were placed in a vial, dissolved in dry DMF (0.949 mL) and then DIPEA (0.084 mL, 0.484 mmol) was added. Reaction was stirred at RT overnight. The reaction mixture was diluted in EtOAc and washed with NaHCO₃. The organic layer was dried MgSO₄, filtered and concentrated to dryness. The crude mixture of tert-butyl 6-chloro-1-(2-(7-(2-((2-(2,6-dioxopiperidin-3-yl)-1,3-dioxoisoindolin-4-yl)oxy)acetyl)-4,7-diazaspiro[2.5]octan-4-yl)ethyl)-3-(3-(naphthalen-1-yloxy)propyl)-7-(1,3,5-trimethyl-1H-pyrazol-4-yl)-1H-indole-2-carboxylate (330.0 mg, crude) was used directly in the next step.

LCMS (ESI⁺): m/z 995.9 [M+H]⁺

### Step F

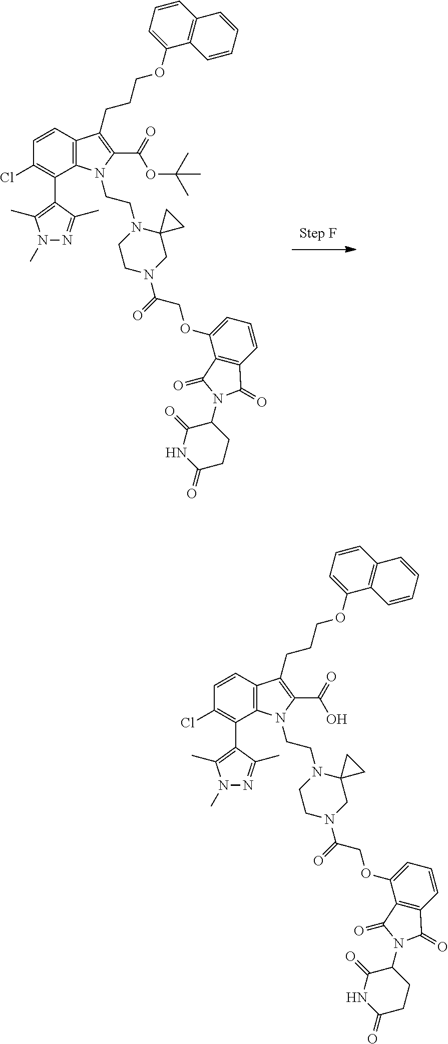 [SMILES: Cc1nn(C)c(C)c1-c1c(Cl)ccc2c(CCCOc3cccc4ccccc34)c(C(=O)OC(C)(C)C)n(CCN3CCN(C(=O)COc4cccc5c4C(=O)N(C4CCC(=O)NC4=O)C5=O)CC34CC4)c12 ; Cc1nn(C)c(C)c1-c1c(Cl)ccc2c(CCCOc3cccc4ccccc34)c(C(=O)O)n(CCN3CCN(C(=O)COc4cccc5c4C(=O)N(C4CCC(=O)NC4=O)C5=O)CC34CC4)c12]

A crude tert-butyl 6-chloro-1-{2-[7-(2-{[2-(2,6-dioxopiperidin-3-yl)-1,3-dioxo-2,3-dihydro-1H-isoindol-4-yl]oxy}acetyl)-4,7-diazaspiro[2.5]octan-4-yl]ethyl}-3-[3-(naphthalen-1-yloxy)propyl]-7-(1,3,5-trimethyl-1H-pyrazol-4-yl)-1H-indole-2-carboxylate (330.0 mg, crude) was dissolved in DCM (1.0 mL), TFA (1.0 mL, 13.246 mmol) was added and reaction was stirred at RT overnight. Solvent was evaporated and crude mixture was purified by preparative HPLC (H₂O:MeCN+0.1% FA) to get a pure product 6-chloro-1-{2-[7-(2-{[2-(2,6-dioxopiperidin-3-yl)-1,3-dioxo-2,3-dihydro-1H-isoindol-4-yl]oxy}acetyl)-4,7-diazaspiro[2.5]octan-4-yl]ethyl}-3-[3-(naphthalen-1-yloxy)propyl]-7-(1,3,5-trimethyl-1H-pyrazol-4-yl)-1H-indole-2-carboxylic acid (6.1 mg, 0.006 mmol, 9.8%) as a beige solid.

LCMS (ESI+): m/z 939.8 [M+H]⁺

¹H NMR (500 MHz, DMSO) δ 10.76 (s, 1H), 8.25-8.21 (m, 1H), 7.87-7.82 (m, 1H), 7.77-7.69 (m, 2H), 7.54-7.47 (m, 2H), 7.46-7.42 (m, 2H), 7.41-7.37 (m, 1H), 7.32 (d, J=8.5 Hz, 1H), 7.21 (d, 1H), 6.92 (d, J=7.6 Hz, 1H), 5.10-4.92 (m, 3H), 4.25 (t, J=6.3 Hz, 2H), 4.18-4.05 (m, 2H), 3.77 (s, 3H), 3.43-3.32 (m, 2H), 3.27 (t, J=7.5 Hz, 2H), 3.11 (s, 2H), 2.89-2.82 (m, 2H), 2.66-2.56 (m, 5H), 2.46-2.42 (m, 3H), 2.29-2.20 (m, 2H), 2.12-1.99 (m, 5H), 1.91 (s, 3H).

### Example 10: 6-chloro-1-{2-[(1S,4S)-5-(2-{[2-(2,6-dioxopiperidin-3-yl)-1-oxo-2,3-dihydro-1H-isoindol-4-yl]oxy}acetyl)-2,5-diazabicyclo[2.2.1]heptan-2-yl]ethyl}-3-[3-(naphthalen-1-yloxy)propyl]-7-(1,3,5-trimethyl-1H-pyrazol-4-yl)-1H-indole-2-carboxylic acid (209)

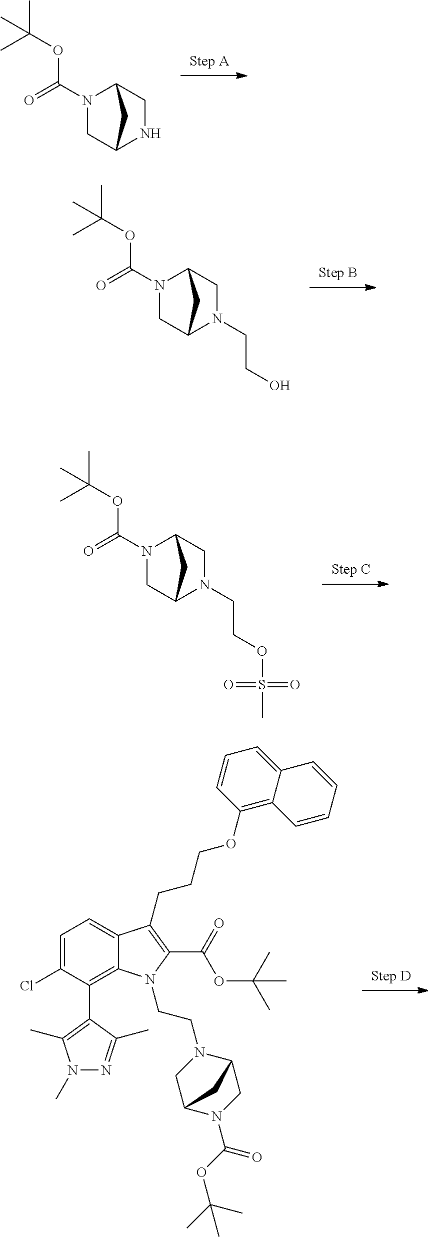

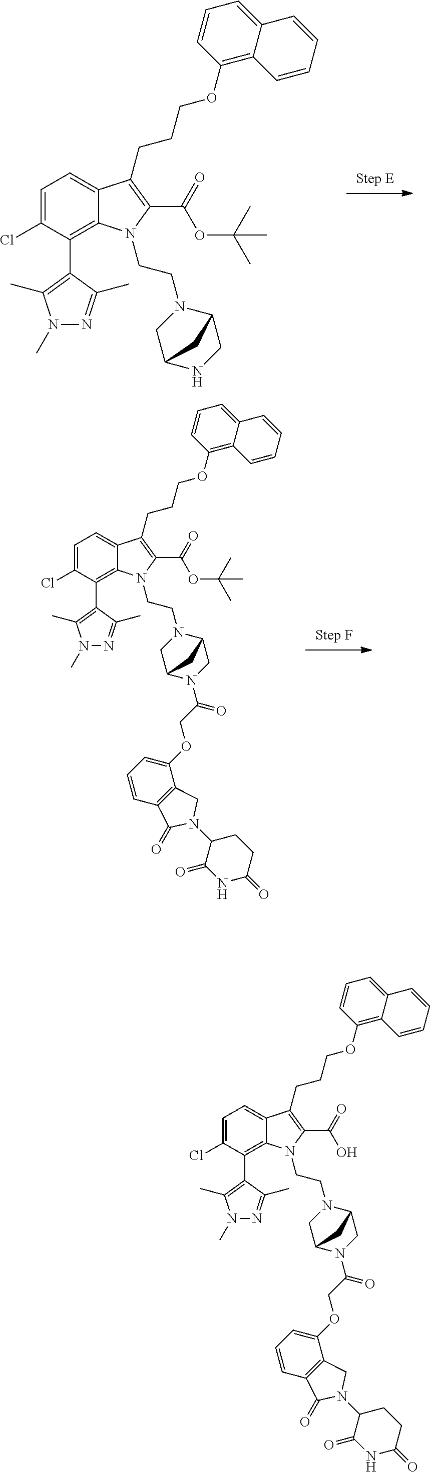 [SMILES: Cc1nn(C)c(C)c1-c1c(Cl)ccc2c(CCCOc3cccc4ccccc34)c(C(=O)OC(C)(C)C)n(CCN3C[C@@H]4C[C@H]3CN4)c12 ; Cc1nn(C)c(C)c1-c1c(Cl)ccc2c(CCCOc3cccc4ccccc34)c(C(=O)OC(C)(C)C)n(CCN3C[C@@H]4C[C@H]3CN4C(=O)COc3cccc4c3CN(C3CCC(=O)NC3=O)C4=O)c12 ; Cc1nn(C)c(C)c1-c1c(Cl)ccc2c(CCCOc3cccc4ccccc34)c(C(=O)O)n(CCN3C[C@@H]4C[C@H]3CN4C(=O)COc3cccc4c3CN(C3CCC(=O)NC3=O)C4=O)c12]

### Step A

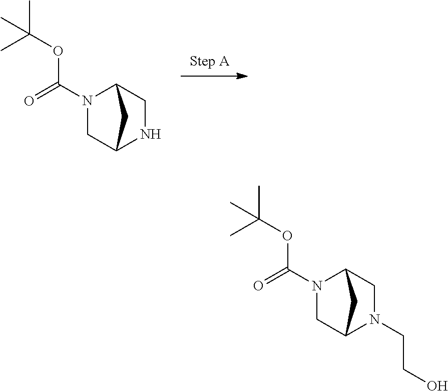 [SMILES: CC(C)(C)OC(=O)N1C[C@@H]2C[C@H]1CN2 ; CC(C)(C)OC(=O)N1C[C@@H]2C[C@H]1CN2CCO]

To a solution of tert-butyl (1S,4S)-2,5-diazabicyclo[2.2.1]heptane-2-carboxylate (0.500 g, 2.522 mmol) in DMF (4.3 mL) under argon was added K₂CO₃ (1.046 g, 7.566 mmol). The mixture was stirred for 2 h at 80° C. After complete consumption of the starting material (monitored by TLC, 10% MeOH in DCM, visualization in ninhydrin), the reaction mixture was cooled down to room temperature and solvents were evaporated under reduced pressure. The resulting residue was dissolved EtOAc and washed with water and brine. Organic layer was dried over Na₂SO₄, filtered and concentrated in vacuo to get crude tert-butyl (1S,4S)-5-(2-hydroxyethyl)-2,5-diazabicyclo[2.2.1]heptane-2-carboxylate (463.5 mg of crude) as pale yellow oil that was used in the next step without further purification.

LCMS (ESI+): 243.2 m/z [M+H]⁺

### Step B

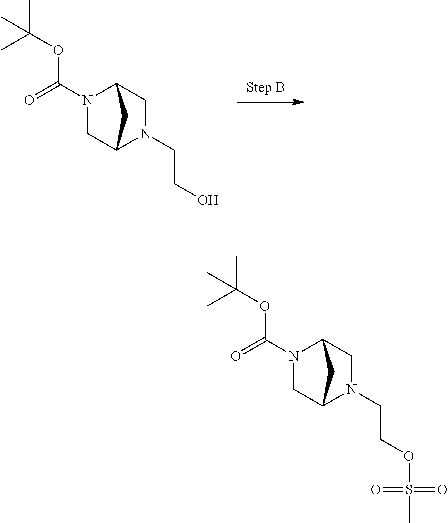 [SMILES: CC(C)(C)OC(=O)N1C[C@@H]2C[C@H]1CN2CCO ; CC(C)(C)OC(=O)N1C[C@@H]2C[C@H]1CN2CCOS(C)(=O)=O]

Tert-butyl (1S,4S)-5-(2-hydroxyethyl)-2,5-diazabicyclo[2.2.1]heptane-2-carboxylate (463.5 mg, crude) was dissolved in DCM (19.1 mL) and Et₃N (0.399 mL, 2.869 mmol), followed by DMAP (23.4 mg, 0.191 mmol) were added. Reaction mixture was cooled down to 0° C. and MsCl (0.178 mL, 2.295 mmol) was added drop-wise. The mixture was allowed to slowly reach RT and stirred for 16 h. After complete consumption of the starting material (monitored with TLC, 20% MeOH in DCM), reaction mixture was diluted with DCM (25 mL) and washed with brine and water. Organic phase was combined, dried over Na₂SO₄, filtered and concentrated in vacuo to give crude (380.5 mg) tert-butyl (1S,4S)-5-[2-(methanesulfonyloxy)ethyl]-2,5-diazabicyclo[2.2.1]heptane-2-carboxylate as yellow oil that was used in the next step without further purification.

LCMS (ESI+): 321.1 m/z [M+H]⁺

### Step C

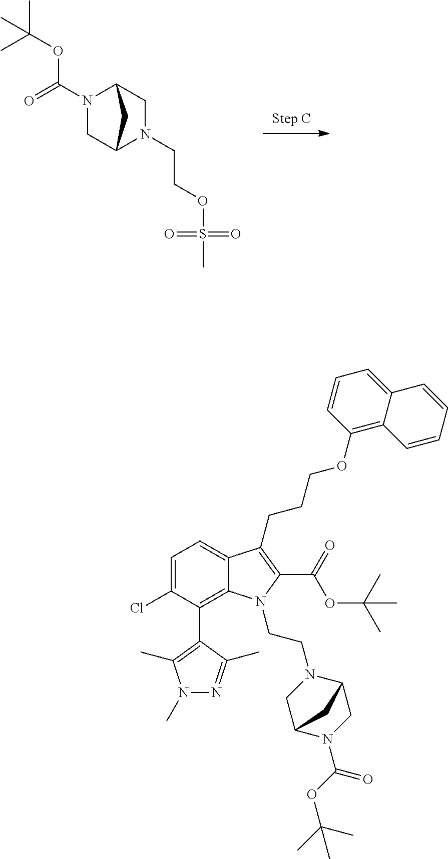 [SMILES: CC(C)(C)OC(=O)N1C[C@@H]2C[C@H]1CN2CCOS(C)(=O)=O ; Cc1nn(C)c(C)c1-c1c(Cl)ccc2c(CCCOc3cccc4ccccc34)c(C(=O)OC(C)(C)C)n(CCN3C[C@@H]4C[C@H]3CN4C(=O)OC(C)(C)C)c12]

Tert-butyl 6-chloro-3-[3-(naphthalen-1-yloxy)propyl]-7-(1,3,5-trimethyl-1H-pyrazol-4-yl)-1H-indole-2-carboxylate (50.0 mg, 0.092 mol) was dissolved in dry DMF (2.0 mL). Cs₂CO₃ (89.8 mg, 0276 mmol) along with tert-butyl (1S,4S)-5-[2-(methanesulfonyloxy)ethyl]-2,5-diazabicyclo[2.2.1]heptane-2-carboxylate (35.3 mg, crude) were added and the reaction was stirred in RT for 18 h. After complete consumption of the starting material (monitored by TLC, (5% MeOH in DCM), and LCMS), the solvent was evaporated under reduced pressure. The resulting residue was dissolved in DCM and washed with water and brine. The organic layer was dried over Na₂SO₄, filtered and concentrated under reduced pressure to give 84.5 mg crude tert-butyl 1-(2-{5-[(tert-butoxy)carbonyl]-2,5-diazabicyclo[2.2.1]heptan-2-yl}ethyl)-6-chloro-3-[3-(naphthalen-1-yloxy)propyl]-7-(1,3,5-trimethyl-1H-pyrazol-4-yl)-1H-indole-2-carboxylate as yellow oil that was used in next step without further purification.

LCMS (ESI+): 768.3 m/z [M+H]⁺

### Step D

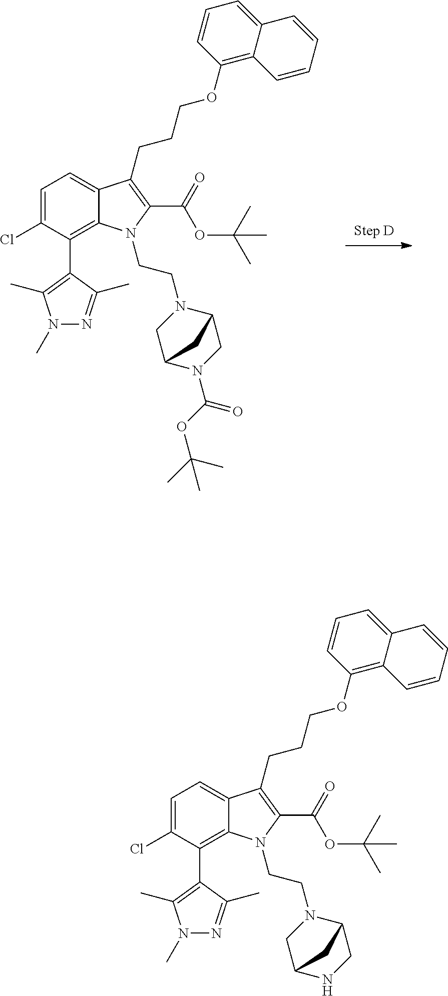 [SMILES: Cc1nn(C)c(C)c1-c1c(Cl)ccc2c(CCCOc3cccc4ccccc34)c(C(=O)OC(C)(C)C)n(CCN3C[C@@H]4C[C@H]3CN4C(=O)OC(C)(C)C)c12 ; Cc1nn(C)c(C)c1-c1c(Cl)ccc2c(CCCOc3cccc4ccccc34)c(C(=O)OC(C)(C)C)n(CCN3C[C@@H]4C[C@H]3CN4)c12]

Tert-butyl 1-(2-{5-[(tert-butoxy)carbonyl]-2,5-diazabicyclo[2.2.1]heptan-2-yl}ethyl)-6-chloro-3-[3-(naphthalen-1-yloxy)propyl]-7-(1,3,5-trimethyl-1H-pyrazol-4-yl)-1H-indole-2-carboxylate (84.5 mg, crude) was dissolved in THE (2.2 mL) under argon atmosphere and cooled down to 0° C. To it, 4M HCl in dioxane (1.4 mL, 5.495 mmol) was added and the reaction (monitored with LCMS) was allowed to reach room temperature (over 4 h). After 16 h of mixing, LCMS analysis indicated full consumption of the starting material. The solution was again cooled down to 0° C. and cool aqueous solution of 1M NaOH was added to pH=7. The solution was diluted in DCM and washed with brine and water. Organic layer was dried over Na₂SO₄, filtered, concentrated and dried under reduced pressure to give 82.0 mg crude tert-butyl 6-chloro-1-(2-{2,5-diazabicyclo[2.2.1]heptan-2-yl}ethyl)-3-[3-(naphthalen-1-yloxy)propyl]-7-(1,3,5-trimethyl-1H-pyrazol-4-yl)-1H-indole-2-carboxylate that was used in the next step without further purification.

LCMS (ESI+): 668.3 m/z [M+H]⁺

### Step E

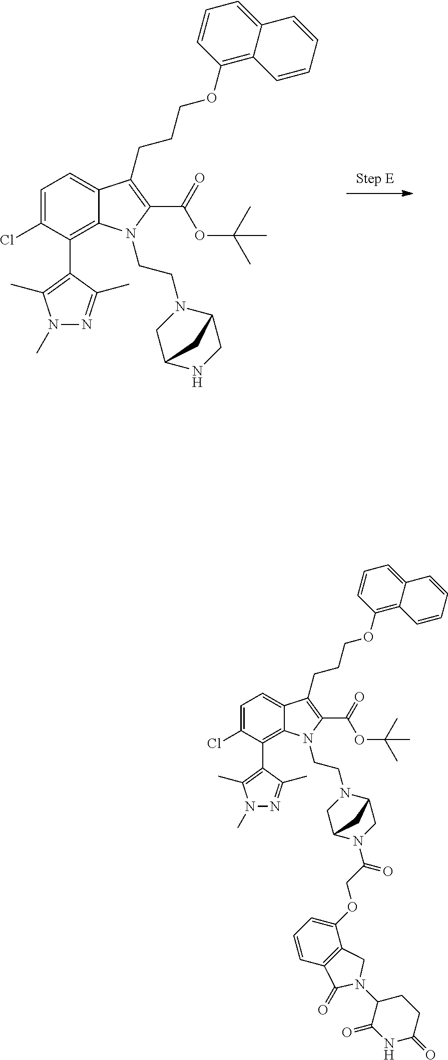 [SMILES: Cc1nn(C)c(C)c1-c1c(Cl)ccc2c(CCCOc3cccc4ccccc34)c(C(=O)OC(C)(C)C)n(CCN3C[C@@H]4C[C@H]3CN4)c12 ; Cc1nn(C)c(C)c1-c1c(Cl)ccc2c(CCCOc3cccc4ccccc34)c(C(=O)OC(C)(C)C)n(CCN3C[C@@H]4C[C@H]3CN4C(=O)COc3cccc4c3CN(C3CCC(=O)NC3=O)C4=O)c12]

2-{[2-(2,6-Dioxopiperidin-3-yl)-1-oxo-2,3-dihydro-1H-isoindol-4-yl]oxy}acetic acid (20.0 mg, 0.063 mmol) was dissolved in DMF (1.3 mL) under argon atmosphere and DIPEA (0.033 mL, 0.189 mmol) and HATU (35.8 mg, 0.094 mmol) were added. Reaction was stirred in room temperature for 15 min and tert-butyl 6-chloro-1-(2-{2,5-diazabicyclo[2.2.1]heptan-2-yl}ethyl)-3-[3-(naphthalen-1-yloxy)propyl]-7-(1,3,5-trimethyl-1H-pyrazol-4-yl)-1H-indole-2-carboxylate (42.0 mg, crude) was added. Reaction was continued in room temperature for 1 h. After consumption of the starting material (monitored with LCMS) solvent was evaporated, resulting residue dissolved in DCM and washed with aqueous NaHCO₃ (saturated), brine and water. Organic layer was collected, dried over Na₂SO₄, filtered, concentrated and dried under reduced pressure to give crude tert-butyl 6-chloro-1-{2-[5-(2-{[2-(2,6-dioxopiperidin-3-yl)-1-oxo-2,3-dihydro-1H-isoindol-4-yl]oxy}acetyl)-2,5-diazabicyclo[2.2.1]heptan-2-yl]ethyl}-3-[3-(naphthalen-1-yloxy)propyl]-7-(1,3,5-trimethyl-1H-pyrazol-4-yl)-1H-indole-2-carboxylate (60.0 mg, crude) as yellow oil, that was used in the next step without further purification.

LCMS (ESI+): 968.3 m/z [M+H]⁺

### Step F

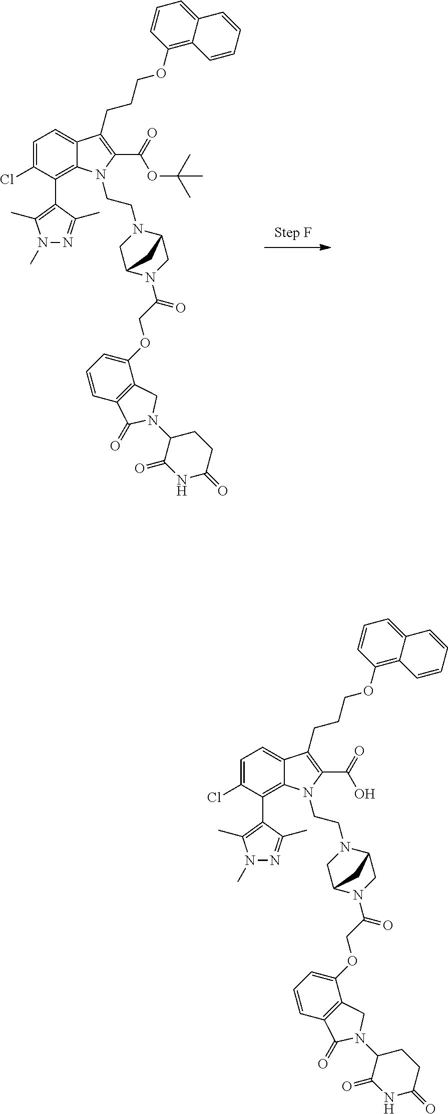 [SMILES: Cc1nn(C)c(C)c1-c1c(Cl)ccc2c(CCCOc3cccc4ccccc34)c(C(=O)OC(C)(C)C)n(CCN3C[C@@H]4C[C@H]3CN4C(=O)COc3cccc4c3CN(C3CCC(=O)NC3=O)C4=O)c12 ; Cc1nn(C)c(C)c1-c1c(Cl)ccc2c(CCCOc3cccc4ccccc34)c(C(=O)O)n(CCN3C[C@@H]4C[C@H]3CN4C(=O)COc3cccc4c3CN(C3CCC(=O)NC3=O)C4=O)c12]

Tert-butyl 6-chloro-1-{2-[5-(2-{[2-(2,6-dioxopiperidin-3-yl)-1-oxo-2,3-dihydro-1H-isoindol-4-yl]oxy}acetyl)-2,5-diazabicyclo[2.2.1]heptan-2-yl]ethyl}-3-[3-(naphthalen-1-yloxy)propyl]-7-(1,3,5-trimethyl-1H-pyrazol-4-yl)-1H-indole-2-carboxylate (60.0 mg, crude) was dissolved in dry DCM (0.4 mL) under argon atmosphere and TFA (0.4 ml, 595.6 mg, 5.224 mmol) was added. Reaction (monitored with LCMS) was stirred in room temperature for 16 h. After full conversion of the starting material, solvent along with TFA was evaporated under reduced pressure, resulting residue dissolved in DMSO and passed through a syringe filter. The filtrate was purified with reverse phase preparative HPLC (H₂O:MeCN+0.1% FA) to give the corresponding 6-chloro-1-{2-[(1S,4S)-5-(2-{[2-(2,6-dioxopiperidin-3-yl)-1-oxo-2,3-dihydro-1H-isoindol-4-yl]oxy}acetyl)-2,5-diazabicyclo[2.2.1]heptan-2-yl]ethyl}-3-[3-(naphthalen-1-yloxy)propyl]-7-(1,3,5-trimethyl-1H-pyrazol-4-yl)-1H-indole-2-carboxylic acid (2.2 mg, 0.002 mmol, 3% over two steps) as white solid.

LCMS (ESI+): 912.1 m/z [M+H]⁺

¹H NMR (500 MHz, DMSO) δ 10.65 (s, 1H), 8.22 (d, J=7.5 Hz, 1H), 7.86 (dd, J=7.5, 1.7 Hz, 1H), 7.70 (d, J=8.4 Hz, 1H), 7.56-7.42 (m, 4H), 7.39 (t, J=7.9 Hz, 1H), 7.34 (d, J=7.6 Hz, 1H), 7.21 (d, J=8.4 Hz, 1H), 7.15 (bs, 1H), 6.91 (d, J=7.6 Hz, 1H), 5.06 (dd, J=12.9, 5.3 Hz, 1H), 4.96-4.65 (m, 2H), 4.51-4.31 (m, 3H), 4.26 (t, J=6.3 Hz, 2H), 4.24-4.07 (m, 2H), 3.82-3.70 (m, 3H), 3.46-3.36 (m, 1H), 3.28 (t, 2H), 3.22-3.14 (m, 2H), 2.93-2.86 (m, 2H), 2.63-2.59 (m, 2H), 2.47-2.45 (m, 1H), 2.37-2.30 (m, 2H), 2.25 (p, J=6.7 Hz, 2H), 2.11-1.97 (m, 4H), 1.90 (d, J=5.5 Hz, 3H), 1.70-1.49 (m, 2H).

### Example 11: 6-chloro-1-(2-{4-[2-({4-[(2,6-dioxopiperidin-3-yl)carbamoyl]-2-methyl-1H-1,3-benzodiazol-6-yl}oxy)acetyl]piperazin-1-yl}ethyl)-3-[3-(naphthalen-1-yloxy)propyl]-7-(1,3,5-trimethyl-1H-pyrazol-4-yl)-1H-indole-2-carboxylic acid (263)

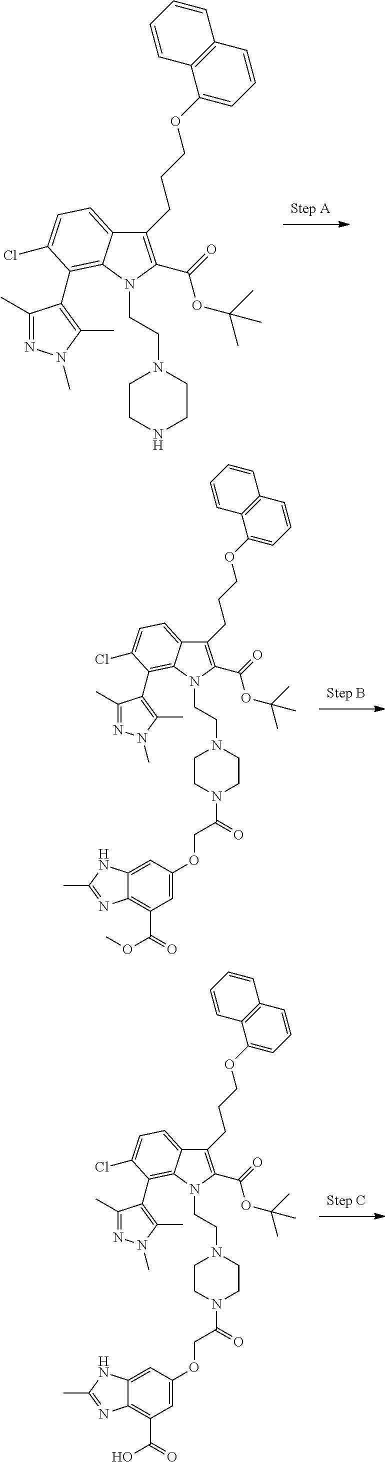

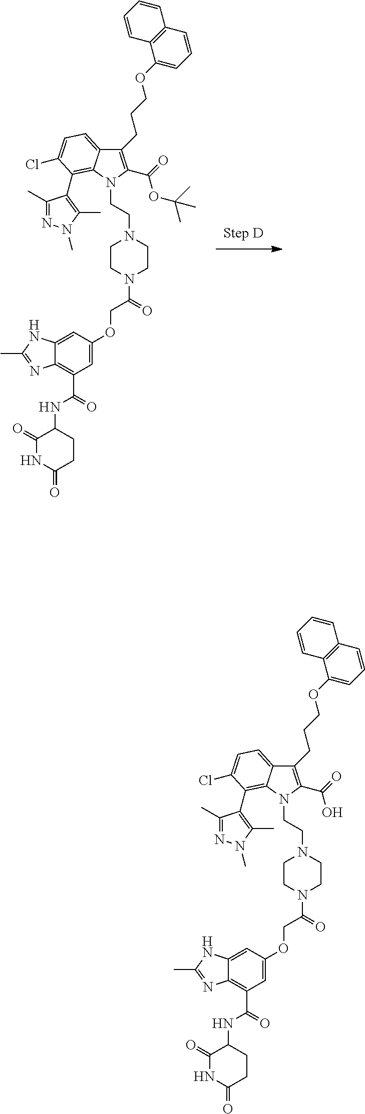 [SMILES: Cc1nc2c(C(=O)NC3CCC(=O)NC3=O)cc(OCC(=O)N3CCN(CCn4c(C(=O)OC(C)(C)C)c(CCCOc5cccc6ccccc56)c5ccc(Cl)c(-c6c(C)nn(C)c6C)c54)CC3)cc2[nH]1 ; Cc1nc2c(C(=O)NC3CCC(=O)NC3=O)cc(OCC(=O)N3CCN(CCn4c(C(=O)O)c(CCCOc5cccc6ccccc56)c5ccc(Cl)c(-c6c(C)nn(C)c6C)c54)CC3)cc2[nH]1]

### Step A

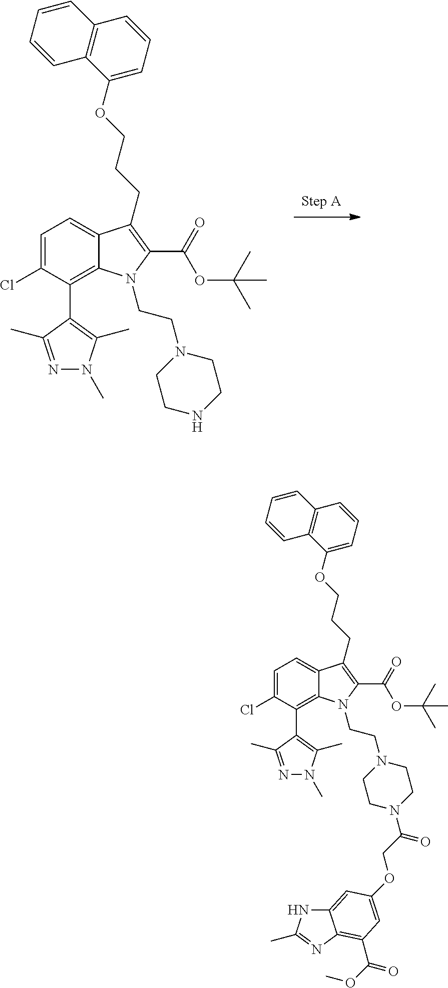 [SMILES: Cc1nn(C)c(C)c1-c1c(Cl)ccc2c(CCCOc3cccc4ccccc34)c(C(=O)OC(C)(C)C)n(CCN3CCNCC3)c12 ; COC(=O)c1cc(OCC(=O)N2CCN(CCn3c(C(=O)OC(C)(C)C)c(CCCOc4cccc5ccccc45)c4ccc(Cl)c(-c5c(C)nn(C)c5C)c43)CC2)cc2[nH]c(C)nc12]

To a well stirred solution of 2-((4-(methoxycarbonyl)-2-methyl-1H-benzo[d]imidazol-6-yl)oxy)acetic acid (24.2 mg, 0.091 mmol) and tert-butyl 6-chloro-3-(3-(naphthalen-1-yloxy)propyl)-1-(2-(piperazin-1-yl)ethyl)-7-(1,3,5-trimethyl-1H-pyrazol-4-yl)-1H-indole-2-carboxylate (50.0 mg, 0.076 mmol) in DMF (0.762 mL) was added DIPEA (0.040 mL, 0.229 mmol) and HATU (34.8 mg, 0.091 mmol) and the mixture was allowed to stir under nitrogen for 16 h. After complete consumption of the starting material solvents were removed under reduced pressure and the reaction mixture diluted with DCM washed successively with cold water (3 times) and brine. Organic layer was dried over Na₂SO₄ and evaporated under reduced pressure to afford methyl 6-{2-[4-(2-{2-[(tert-butoxy)carbonyl]-6-chloro-3-[3-(naphthalen-1-yloxy)propyl]-7-(1,3,5-trimethyl-1H-pyrazol-4-yl)-1H-indol-1-yl}ethyl)piperazin-1-yl]-2-oxoethoxy}-2-methyl-1H-1,3-benzodiazole-4-carboxylate (40.0 mg crude) as brown solid which was used for next step without further purification.

LCMS (ESI+): m/z 902.5 [M+H]⁺

### Step G

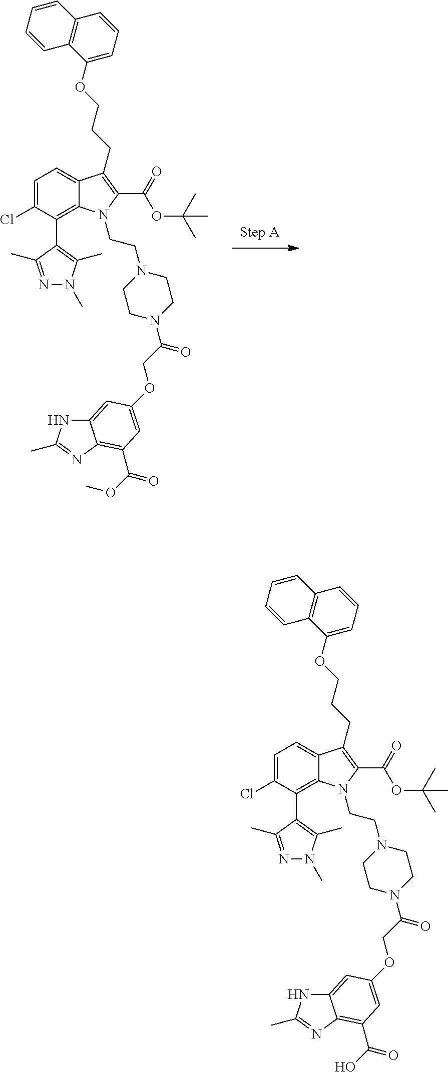 [SMILES: COC(=O)c1cc(OCC(=O)N2CCN(CCn3c(C(=O)OC(C)(C)C)c(CCCOc4cccc5ccccc45)c4ccc(Cl)c(-c5c(C)nn(C)c5C)c43)CC2)cc2[nH]c(C)nc12 ; Cc1nc2c(C(=O)O)cc(OCC(=O)N3CCN(CCn4c(C(=O)OC(C)(C)C)c(CCCOc5cccc6ccccc56)c5ccc(Cl)c(-c6c(C)nn(C)c6C)c54)CC3)cc2[nH]1]

Methyl 6-{2-[4-(2-{2-[(tert-butoxy)carbonyl]-6-chloro-3-[3-(naphthalen-1-yloxy)propyl]-7-(1,3,5-trimethyl-1H-pyrazol-4-yl)-1H-indol-1-yl}ethyl)piperazin-1-yl]-2-oxoethoxy}-2-methyl-1H-1,3-benzodiazole-4-carboxylate (40.0 mg crude) was dissolved in MeOH (4.4 mL) and few drops of DCM were added for solubility. 1M LiOH (222 μL, 0.222 mmol) was added, and mixture was stirred in RT for overnight. To mixture water and 1M HCl were added till pH ˜3 and mixture was extracted with DCM. 6-{2-[4-(2-{2-[(tert-butoxy)carbonyl]-6-chloro-3-[3-(naphthalen-1-yloxy)propyl]-7-(1,3,5-trimethyl-1H-pyrazol-4-yl)-1H-indol-1-yl}ethyl)piperazin-1-yl]-2-oxoethoxy}-2-methyl-1H-1,3-benzodiazole-4-carboxylic acid (25.0 mg of crude) was obtained as brow gum, which was used for next step without further purification.

LCMS (ESI+): m/z 887.5 [M+H]⁺

### Step C

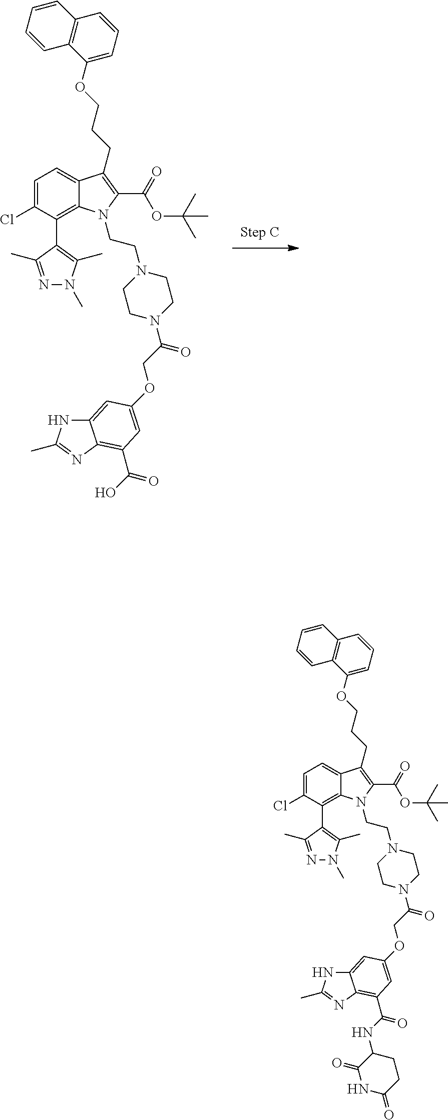 [SMILES: Cc1nc2c(C(=O)O)cc(OCC(=O)N3CCN(CCn4c(C(=O)OC(C)(C)C)c(CCCOc5cccc6ccccc56)c5ccc(Cl)c(-c6c(C)nn(C)c6C)c54)CC3)cc2[nH]1 ; Cc1nc2c(C(=O)NC3CCC(=O)NC3=O)cc(OCC(=O)N3CCN(CCn4c(C(=O)OC(C)(C)C)c(CCCOc5cccc6ccccc56)c5ccc(Cl)c(-c6c(C)nn(C)c6C)c54)CC3)cc2[nH]1]

To a well stirred solution of 3-aminopiperidine-2,6-dione (3.7 mg, 0.023 mmol) and 6-{2-[4-(2-{2-[(tert-butoxy)carbonyl]-6-chloro-3-[3-(naphthalen-1-yloxy)propyl]-7-(1,3,5-trimethyl-1H-pyrazol-4-yl)-1H-indol-1-yl}ethyl)piperazin-1-yl]-2-oxoethoxy}-2-methyl-1H-1,3-benzodiazole-4-carboxylic acid (10.0 mg, crude) in DMF (0.113 mL) was added DIPEA (0.006 mL, 0.034 mmol) and HATU (4.3 mg, 0.011 mmol) and the mixture was allowed to stir under nitrogen for 16 h. After complete consumption of the starting material the reaction mixture diluted with DCM, washed successively with brine. Organic layer was dried over Na₂SO₄ and evaporated under reduced pressure. Mixture was purified using flash chromatography (SiO₂, 7% MeOH in DCM) to afford tert-butyl 6-chloro-1-(2-{4-[2-({4-[(2,6-dioxopiperidin-3-yl)carbamoyl]-2-methyl-1H-1,3-benzodiazol-6-yl}oxy)acetyl]piperazin-1-yl}ethyl)-3-[3-(naphthalen-1-yloxy)propyl]-7-(1,3,5-trimethyl-1H-pyrazol-4-yl)-1H-indole-2-carboxylate (11.5 mg, 0.010 mmol, 57% over two steps) as brown semisolid.

LCMS (ESI+): m/z 998.4 [M+H]⁺

### Step D

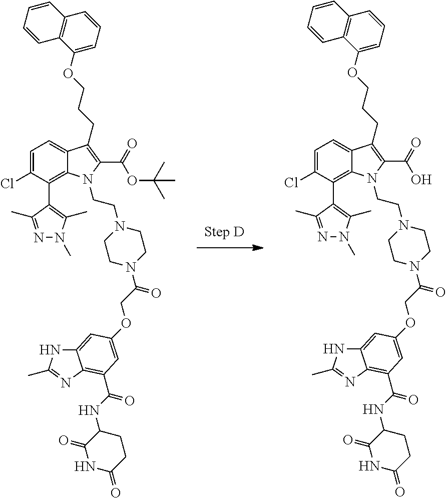 [SMILES: Cc1nc2c(C(=O)NC3CCC(=O)NC3=O)cc(OCC(=O)N3CCN(CCn4c(C(=O)OC(C)(C)C)c(CCCOc5cccc6ccccc56)c5ccc(Cl)c(-c6c(C)nn(C)c6C)c54)CC3)cc2[nH]1 ; Cc1nc2c(C(=O)NC3CCC(=O)NC3=O)cc(OCC(=O)N3CCN(CCn4c(C(=O)O)c(CCCOc5cccc6ccccc56)c5ccc(Cl)c(-c6c(C)nn(C)c6C)c54)CC3)cc2[nH]1]

Tert-butyl 6-chloro-1-(2-{4-[2-({4-[(2,6-dioxopiperidin-3-yl)carbamoyl]-2-methyl-1H-1,3-benzodiazol-6-yl}oxy)acetyl]piperazin-1-yl}ethyl)-3-[3-(naphthalen-1-yloxy)propyl]-7-(1,3,5-trimethyl-1H-pyrazol-4-yl)-1H-indole-2-carboxylate (11.0 mg, 0.011 mmol) was dissolved in DCM (0.110 mL), and TFA (0.017 mL, 0.220 mmol) was added. Mixture was stirred for 4 h, then solvents were removed under reduced pressure, and crude was purified using preparative HPLC (H₂O:MeCN+0.1% FA) to get 6-chloro-1-(2-{4-[2-({4-[(2,6-dioxopiperidin-3-yl)carbamoyl]-2-methyl-1H-1,3-benzodiazol-6-yl}oxy)acetyl]piperazin-1-yl}ethyl)-3-[3-(naphthalen-1-yloxy)propyl]-7-(1,3,5-trimethyl-1H-pyrazol-4-yl)-1H-indole-2-carboxylic acid (1.7 mg, 0.002 mmol, 16.4%) as white solid.

LCMS (ESI+): m/z 942.9 [M+H]⁺

¹H NMR (500 MHz, DMSO) δ 12.79-12.70 (m, 1H), 12.55 (s, 1H), 10.92 (s, 1H), 10.26 (d, J=7.3 Hz, 1H), 8.26-8.19 (m, 1H), 7.92-7.84 (m, 1H), 7.77 (d, J=8.6 Hz, 1H), 7.57-7.48 (m, 2H), 7.46 (d, J=8.3 Hz, 1H), 7.40 (t, J=7.9 Hz, 2H), 7.24 (d, J=8.5 Hz, 1H), 7.17 (s, 1H), 6.97-6.88 (m, 1H), 4.91-4.79 (m, 3H), 4.36-4.26 (m, 1H), 4.26-4.13 (m, 3H), 3.76 (s, 3H), 3.47-3.35 (m, 5H), 3.30-3.26 (m, 3H), 2.90-2.76 (m, 1H), 2.62-2.55 (m, 2H), 2.28-2.19 (m, 3H), 2.19-1.97 (m, 10H), 1.89 (s, 3H).

### Example 12: 6-chloro-1-(2-(4-(2-((4-((2,6-dioxopiperidin-3-yl)carbamoyl)-2-methyl-1H-benzo[d]imidazol-5-yl)oxy)acetyl)piperazin-1-yl)ethyl)-3-(3-(naphthalen-1-yloxy)propyl)-7-(1,3,5-trimethyl-1H-pyrazol-4-yl)-1H-indole-2-carboxylic acid (264)

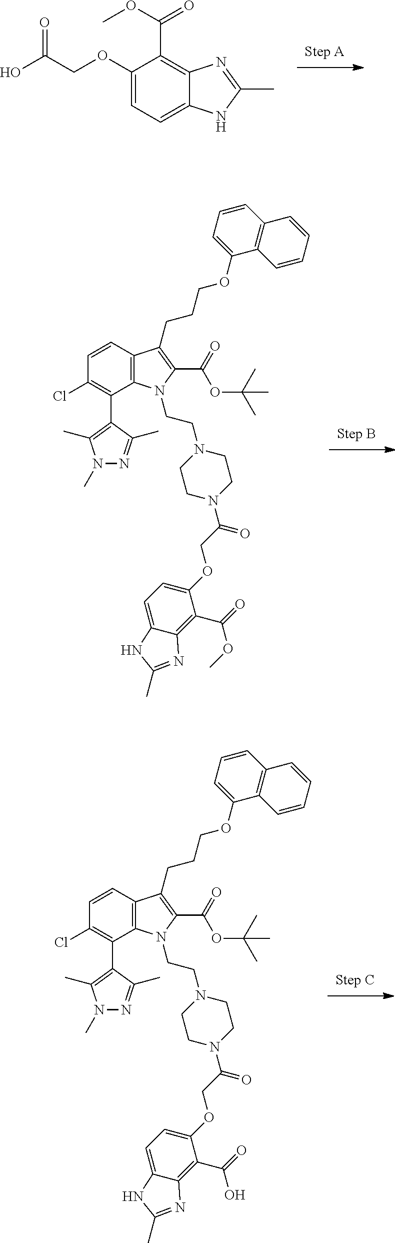

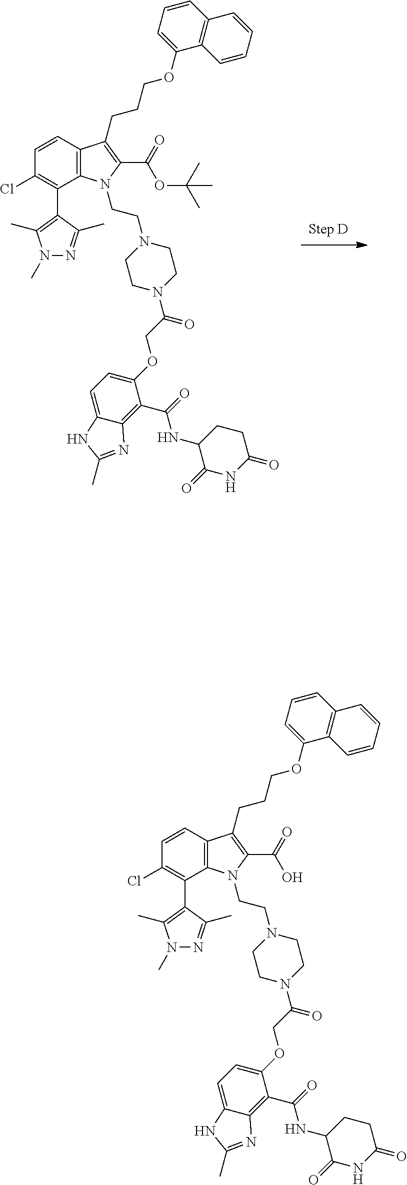 [SMILES: Cc1nc2c(C(=O)NC3CCC(=O)NC3=O)c(OCC(=O)N3CCN(CCn4c(C(=O)OC(C)(C)C)c(CCCOc5cccc6ccccc56)c5ccc(Cl)c(-c6c(C)nn(C)c6C)c54)CC3)ccc2[nH]1 ; Cc1nc2c(C(=O)NC3CCC(=O)NC3=O)c(OCC(=O)N3CCN(CCn4c(C(=O)O)c(CCCOc5cccc6ccccc56)c5ccc(Cl)c(-c6c(C)nn(C)c6C)c54)CC3)ccc2[nH]1]

### Step A

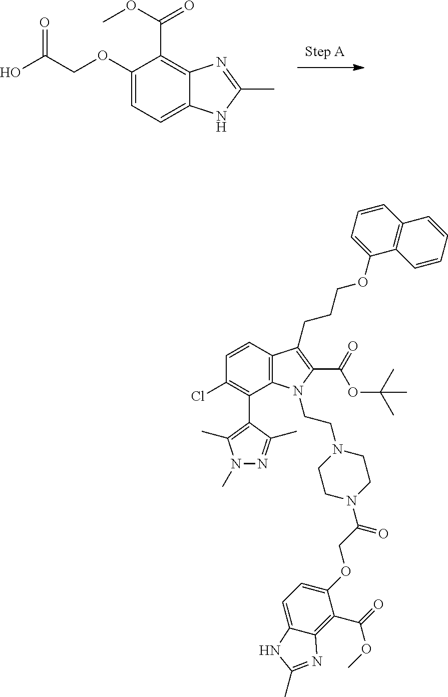 [SMILES: COC(=O)c1c(OCC(=O)O)ccc2[nH]c(C)nc12 ; COC(=O)c1c(OCC(=O)N2CCN(CCn3c(C(=O)OC(C)(C)C)c(CCCOc4cccc5ccccc45)c4ccc(Cl)c(-c5c(C)nn(C)c5C)c43)CC2)ccc2[nH]c(C)nc12]

2-((4-(methoxycarbonyl)-2-methyl-1H-benzo[d]imidazol-5-yl)oxy)acetic acid (15.0 mg, 0.057 mmol) was dissolved in dry DMF (1.1 mL) under argon atmosphere. DIPEA (0.030 mL, 0.170 mmol), followed by HATU (32.4 mg, 0.085 mmol) were added and the solution was stirred for 15 min in room temperature. To the mixture was added tert-butyl 6-chloro-3-(3-(naphthalen-1-yloxy)propyl)-1-(2-(piperazin-1-yl)ethyl)-7-(1,3,5-trimethyl-1H-pyrazol-4-yl)-1H-indole-2-carboxylate (37.3 mg, 0.057 mmol) and the reaction was stirred in room temperature. Reaction progress was monitored with LCMS. After 90 min starting material was still present (monitored by LCMS) in the reaction mixture. Additional portion of 2-{[4-(methoxycarbonyl)-2-methyl-1H-1,3-benzodiazol-5-yl]oxy}acetic acid (1.5 mg, 0.006 mmol) and HATU (3.2 mg, 0.008 mmol) were added and the reaction was continued in room temperature. After next 60 min DMF was evaporated, resulting residue dissolved in EtOAc (30 ml) and washed with brine (2×20 ml) and water (20 ml). Organic layer was dried over Na₂SO₄, filtered and dried under reduced pressure to give 50 mg crude methyl 5-{2-[4-(2-{2-[(tert-butoxy)carbonyl]-6-chloro-3-[3-(naphthalen-1-yloxy)propyl]-7-(1,3,5-trimethyl-1H-pyrazol-4-yl)-1H-indol-1-yl}ethyl)piperazin-1-yl]-2-oxoethoxy}-2-methyl-1H-1,3-benzodiazole-4-carboxylate as dark yellow sticky solid that was used in the next step without further purification.

LCMS (ESI+): m/z 902.8 [M+H]⁺

### Step B

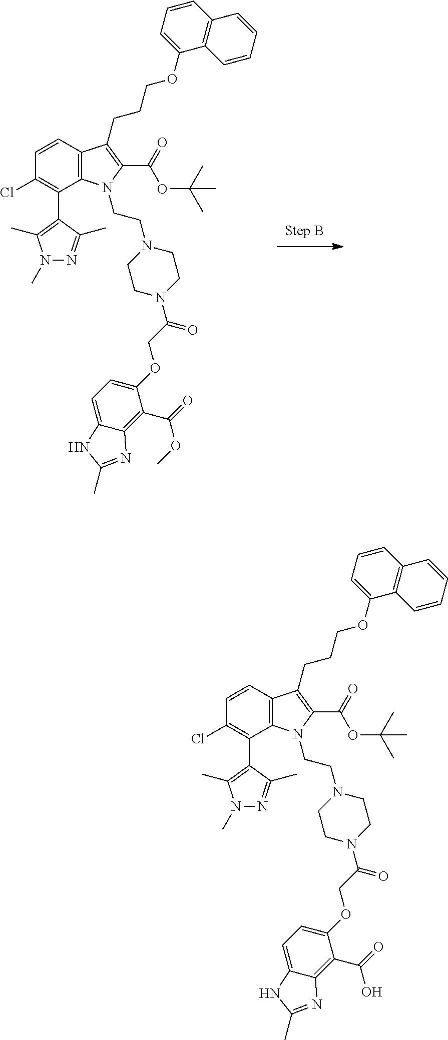 [SMILES: COC(=O)c1c(OCC(=O)N2CCN(CCn3c(C(=O)OC(C)(C)C)c(CCCOc4cccc5ccccc45)c4ccc(Cl)c(-c5c(C)nn(C)c5C)c43)CC2)ccc2[nH]c(C)nc12 ; Cc1nc2c(C(=O)O)c(OCC(=O)N3CCN(CCn4c(C(=O)OC(C)(C)C)c(CCCOc5cccc6ccccc56)c5ccc(Cl)c(-c6c(C)nn(C)c6C)c54)CC3)ccc2[nH]1]

To a solution of methyl 5-{2-[4-(2-{2-[(tert-butoxy)carbonyl]-6-chloro-3-[3-(naphthalen-1-yloxy)propyl]-7-(1,3,5-trimethyl-1H-pyrazol-4-yl)-1H-indol-1-yl}ethyl)piperazin-1-yl]-2-oxoethoxy}-2-methyl-1H-1,3-benzodiazole-4-carboxylate (50 mg, crude) in MeCN (3.0 mL), was added LiBr (192.4 mg, 2.216 mmol) in water (0.2 ml), followed by Et₃N (0.046 mL, 0.332 mmol) and the reaction was stirred for 36 h at 60° C. After that time, the addition of LiBr (192.4 mg, 2.216 mmol) and Et₃N (0.046 mL, 0.332 mmol) was repeated and the reaction stirred for the next 36 h at 60° C. After that time LCMS indicated full conversion. Solvents along with Et₃N were evaporated under reduced pressure, crude product dissolved in EtOAc and washed with water. EtOAc was evaporated and reaction product dried under vacuo to give 44.5 mg crude tert-butyl 6-chloro-1-(2-{4-[2-({4-[(2,6-dioxopiperidin-3-yl)carbamoyl]-2-methyl-1H-1,3-benzodiazol-5-yl}oxy)acetyl]piperazin-1-yl}ethyl)-3-[3-(naphthalen-1-yloxy)propyl]-7-(1,3,5-trimethyl-1H-pyrazol-4-yl)-1H-indole-2-carboxylate as brown solid that was used in the next step without further purification.

LCMS (ESI+): m/z 888.3 [M+H]⁺

### Step C

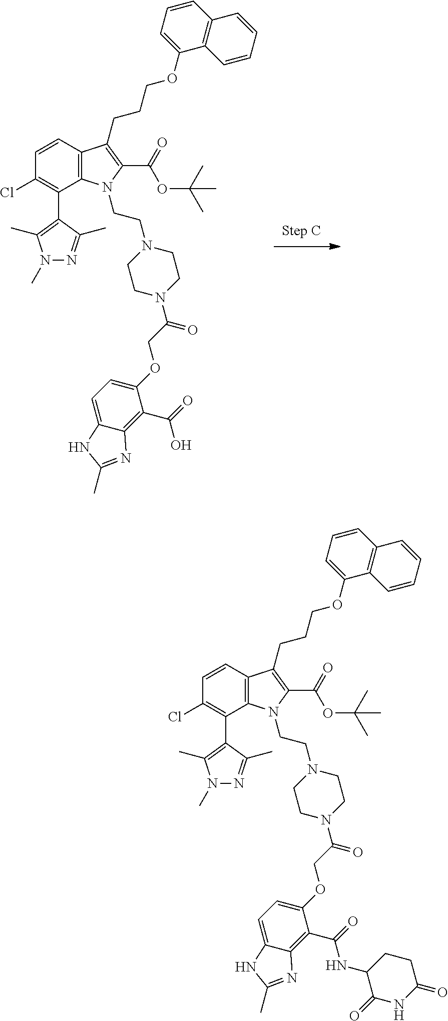 [SMILES: Cc1nc2c(C(=O)O)c(OCC(=O)N3CCN(CCn4c(C(=O)OC(C)(C)C)c(CCCOc5cccc6ccccc56)c5ccc(Cl)c(-c6c(C)nn(C)c6C)c54)CC3)ccc2[nH]1 ; Cc1nc2c(C(=O)NC3CCC(=O)NC3=O)c(OCC(=O)N3CCN(CCn4c(C(=O)OC(C)(C)C)c(CCCOc5cccc6ccccc56)c5ccc(Cl)c(-c6c(C)nn(C)c6C)c54)CC3)ccc2[nH]1]

To a solution of crude 5-(2-(4-(2-(2-(tert-butoxycarbonyl)-6-chloro-3-(3-(naphthalen-1-yloxy)propyl)-7-(1,3,5-trimethyl-1H-pyrazol-4-yl)-1H-indol-1-yl)ethyl)piperazin-1-yl)-2-oxoethoxy)-2-methyl-1H-1,3-benzodiazole-4-carboxylic acid (44.5 mg) in dry DMF (1.0 mL), was added DIPEA (0.026 mL, 0.150 mmol), followed by HATU (28.6 mg, 0.075 mmol) and solution was stirred at room temperature. After 15 min 3-aminopiperidine-2,6-dione hydrochloride (12.4 mg, 0.075 mmol) was added and the reaction was stirred for 4 h at room temperature. DMF was evaporated, residues were dissolved in EtOAc and washed 3× with water. Solvents were removed under reduced pressure and reaction product dried overnight under reduced pressure to give crude (45.1 mg) tert-butyl 6-chloro-1-(2-{4-[2-({4-[(2,6-dioxopiperidin-3-yl)carbamoyl]-2-methyl-1H-1,3-benzodiazol-5-yl}oxy)acetyl]piperazin-1-yl}ethyl)-3-[3-(naphthalen-1-yloxy)propyl]-7-(1,3,5-trimethyl-1H-pyrazol-4-yl)-1H-indole-2-carboxylate as yellow oil, which was used to the next step without further purification.

LCMS (ESI+): m/z 998.1 [M+H]⁺

### Step D

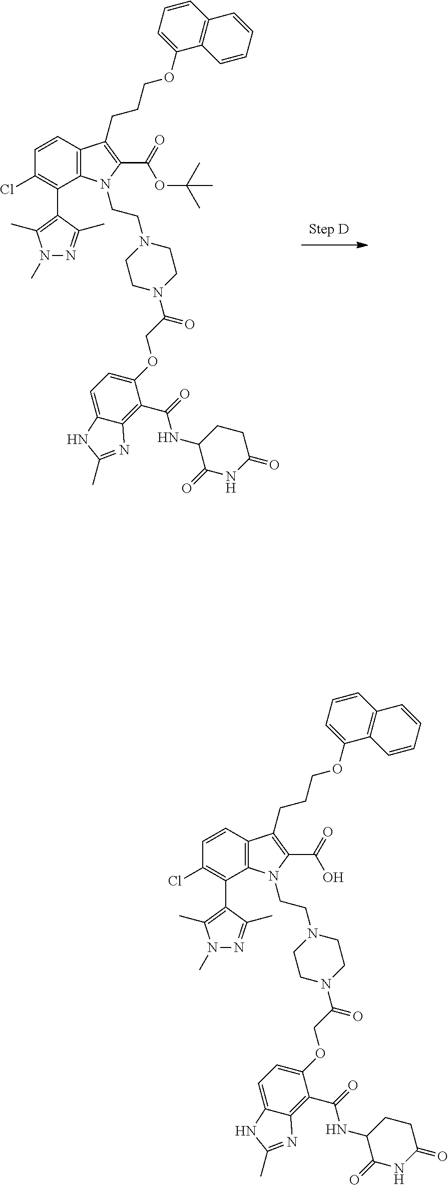 [SMILES: Cc1nc2c(C(=O)NC3CCC(=O)NC3=O)c(OCC(=O)N3CCN(CCn4c(C(=O)OC(C)(C)C)c(CCCOc5cccc6ccccc56)c5ccc(Cl)c(-c6c(C)nn(C)c6C)c54)CC3)ccc2[nH]1 ; Cc1nc2c(C(=O)NC3CCC(=O)NC3=O)c(OCC(=O)N3CCN(CCn4c(C(=O)O)c(CCCOc5cccc6ccccc56)c5ccc(Cl)c(-c6c(C)nn(C)c6C)c54)CC3)ccc2[nH]1]

The crude tert-butyl 6-chloro-1-(2-{4-[2-({4-[(2,6-dioxopiperidin-3-yl)carbamoyl]-2-methyl-1H-1,3-benzodiazol-5-yl}oxy)acetyl]piperazin-1-yl}ethyl)-3-[3-(naphthalen-1-yloxy)propyl]-7-(1,3,5-trimethyl-1H-pyrazol-4-yl)-1H-indole-2-carboxylate (45.1 mg) was dissolved in dry DCM (0.350 mL) under argon atmosphere and TFA (0.346 mL, 4.515 mmol) was added. Reaction mixture was stirred at room temperature for 48 h. Reaction mixture was concentrated and dried under reduced pressure and the resulting solid dissolved in DMSO and purified twice by preparative HPLC (H₂O:MeCN+0.1% FA) to give 6-chloro-1-(2-(4-(2-((4-((2,6-dioxopiperidin-3-yl)carbamoyl)-2-methyl-1H-benzo[d]imidazol-5-yl)oxy)acetyl)piperazin-1-yl)ethyl)-3-(3-(naphthalen-1-yloxy)propyl)-7-(1,3,5-trimethyl-1H-pyrazol-4-yl)-1H-indole-2-carboxylic acid (2.7 mg, 0.003 mmol, 5% yield over four steps) as white solid.

LCMS (ESI+): m/z 942.5 [M+H]⁺

¹H NMR (500 MHz, DMSO) δ 13.42 (s, 1H), 12.11 (s, 1H), 10.85 (d, J=5.2 Hz, 1H), 10.04 (d, J=8.1 Hz, 1H), 8.26-8.18 (m, 1H), 7.90-7.83 (m, 1H), 7.79-7.73 (m, 1H), 7.64 (d, J=8.7 Hz, 1H), 7.56-7.49 (m, 2H), 7.46 (d, J=8.2 Hz, 1H), 7.40 (t, J=7.9 Hz, 1H), 7.28-7.20 (m, 1H), 7.02 (d, J=8.8 Hz, 1H), 6.92 (d, J=7.4 Hz, 1H), 5.02 (s, 2H), 4.79 (ddd, J=12.0, 8.1, 5.5 Hz, 1H), 4.34-4.16 (m, 4H), 3.78 (s, 3H), 3.41-3.35 (m, 5H), 3.30 (s, 3H), 2.88-2.80 (m, 1H), 2.61-2.59 (m, 2H), 2.29-2.17 (m, 3H), 2.16-2.11 (m, 3H), 2.09-1.98 (m, 7H), 1.90 (s, 3H).

### Example 13: 6-chloro-1-{2-[4-(3-{4-[(2,6-dioxopiperidin-3-yl)carbamoyl]-1H-1,3-benzodiazol-2-yl}propanoyl)piperazin-1-yl]ethyl}-3-f 3-(naphthalen-1-yloxy)propyl]-7-(1,3,5-trimethyl-1H-pyrazol-4-yl)-1H-indole-2-carboxylic acid (265)

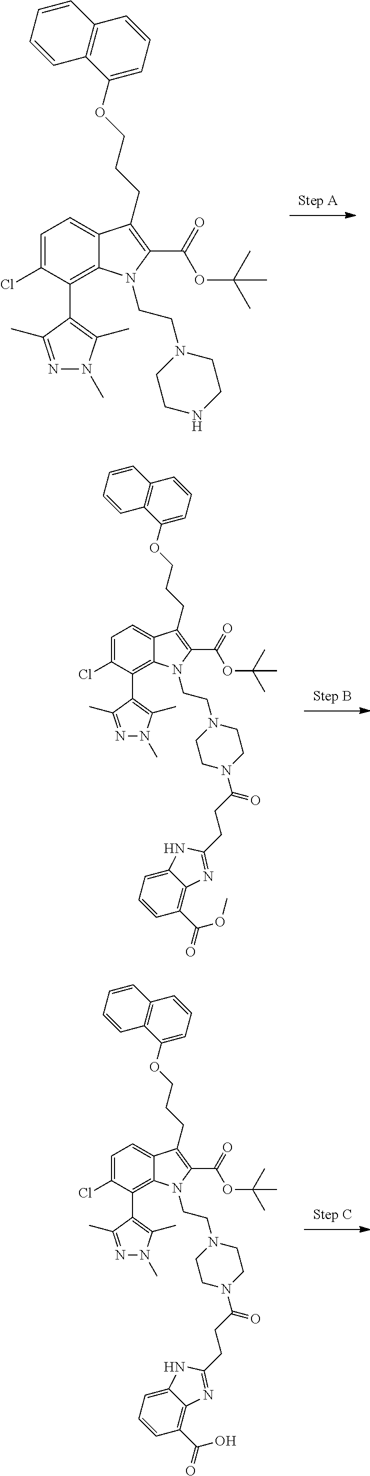

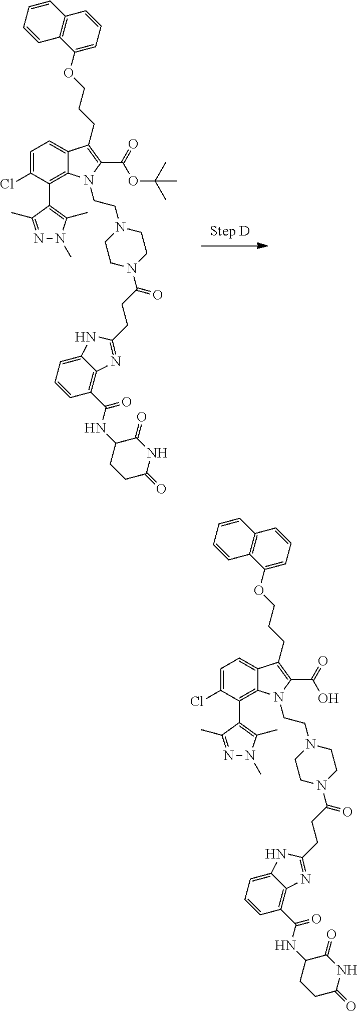 [SMILES: Cc1nn(C)c(C)c1-c1c(Cl)ccc2c(CCCOc3cccc4ccccc34)c(C(=O)OC(C)(C)C)n(CCN3CCN(C(=O)CCc4nc5c(C(=O)NC6CCC(=O)NC6=O)cccc5[nH]4)CC3)c12 ; Cc1nn(C)c(C)c1-c1c(Cl)ccc2c(CCCOc3cccc4ccccc34)c(C(=O)O)n(CCN3CCN(C(=O)CCc4nc5c(C(=O)NC6CCC(=O)NC6=O)cccc5[nH]4)CC3)c12]

### Step A

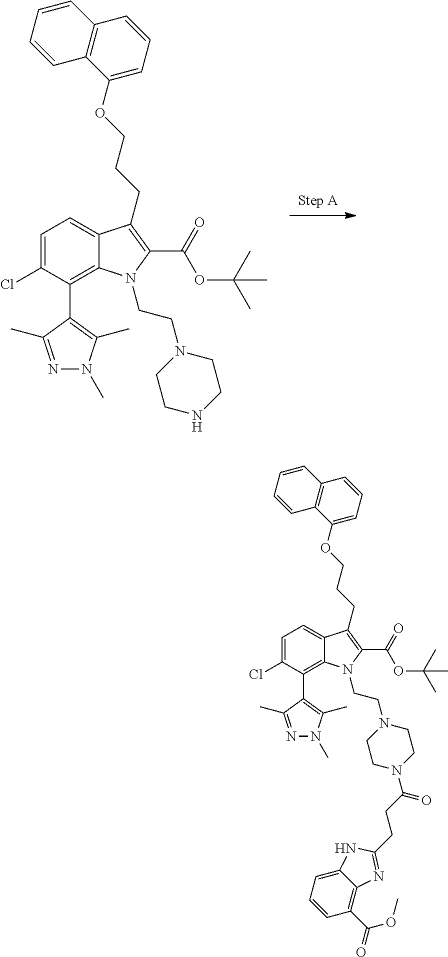 [SMILES: Cc1nn(C)c(C)c1-c1c(Cl)ccc2c(CCCOc3cccc4ccccc34)c(C(=O)OC(C)(C)C)n(CCN3CCNCC3)c12 ; COC(=O)c1cccc2[nH]c(CCC(=O)N3CCN(CCn4c(C(=O)OC(C)(C)C)c(CCCOc5cccc6ccccc56)c5ccc(Cl)c(-c6c(C)nn(C)c6C)c54)CC3)nc12]

To a well stirred solution of 3-(7-(methoxycarbonyl)-1H-benzo[d]imidazol-2-yl)propanoic acid (21.2 mg, 0.084 mmol) and tert-butyl 6-chloro-3-(3-(naphthalen-1-yloxy)propyl)-1-(2-(piperazin-1-yl)ethyl)-7-(1,3,5-trimethyl-1H-pyrazol-4-yl)-1H-indole-2-carboxylate (50.0 mg, 0.076 mmol) in DMF (0.762 mL) was added DIPEA (0.040 mL, 0.229 mmol) and HATU (34.8 mg, 0.091 mmol) and the mixture was allowed to stir under nitrogen for 16 h. After complete consumption of the starting material, solvents were removed under reduced pressure and the reaction mixture diluted with DCM washed successively with NaHCO₃ (sat.) and brine. Organic layer was dried over Na₂SO₄ and evaporated under reduced pressure to afford methyl 2-{3-[4-(2-{2-[(tert-butoxy)carbonyl]-6-chloro-3-[3-(naphthalen-1-yloxy)propyl]-7-(1,3,5-trimethyl-1H-pyrazol-4-yl)-1H-indol-1-yl}ethyl)piperazin-1-yl]-3-oxopropyl}-1H-1,3-benzodiazole-4-carboxylate (55 mg, crude) as brown semi solid which was used for next step without further purification.

LCMS (ESI+): m/z 886.1 [M+H]⁺

### Step B

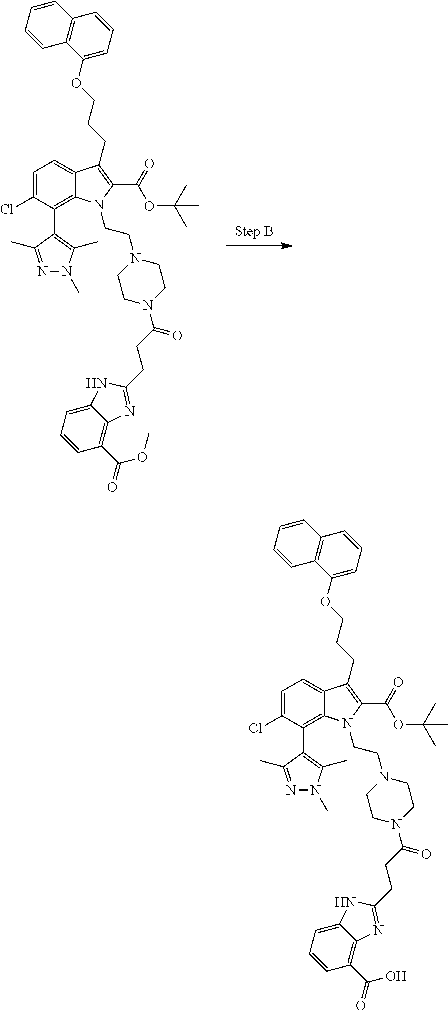 [SMILES: COC(=O)c1cccc2[nH]c(CCC(=O)N3CCN(CCn4c(C(=O)OC(C)(C)C)c(CCCOc5cccc6ccccc56)c5ccc(Cl)c(-c6c(C)nn(C)c6C)c54)CC3)nc12 ; Cc1nn(C)c(C)c1-c1c(Cl)ccc2c(CCCOc3cccc4ccccc34)c(C(=O)OC(C)(C)C)n(CCN3CCN(C(=O)CCc4nc5c(C(=O)O)cccc5[nH]4)CC3)c12]

Methyl 2-{3-[4-(2-{2-[(tert-butoxy)carbonyl]-6-chloro-3-[3-(naphthalen-1-yloxy)propyl]-7-(1,3,5-trimethyl-1H-pyrazol-4-yl)-1H-indol-1-yl}ethyl)piperazin-1-yl]-3-oxopropyl}-1H-1,3-benzodiazole-4-carboxylate (55.0 mg) was dissolved in THE (0.472 mL) and H₂O (0.472 mL). LiOH (0.236 mL, 0.236 mmol) was added, and mixture was stirred in room temperature for 4 days. In the meantime precipitate was observed, and THE was added for solubility. After reaction was finished THE was removed under reduced pressure and to mixture water and 1M HCl were added till pH ˜3 and mixture was extracted with DCM. 2-{3-[4-(2-{2-[(tert-butoxy)carbonyl]-6-chloro-3-[3-(naphthalen-1-yloxy)propyl]-7-(1,3,5-trimethyl-1H-pyrazol-4-yl)-1H-indol-1-yl}ethyl)piperazin-1-yl]-3-oxopropyl}-1H-1,3-benzodiazole-4-carboxylic acid (23.0 mg, crude) was obtained as brown gum, which was used for next step without further purification.

LCMS (ESI+): m/z 872.0 [M+H]⁺

### Step C

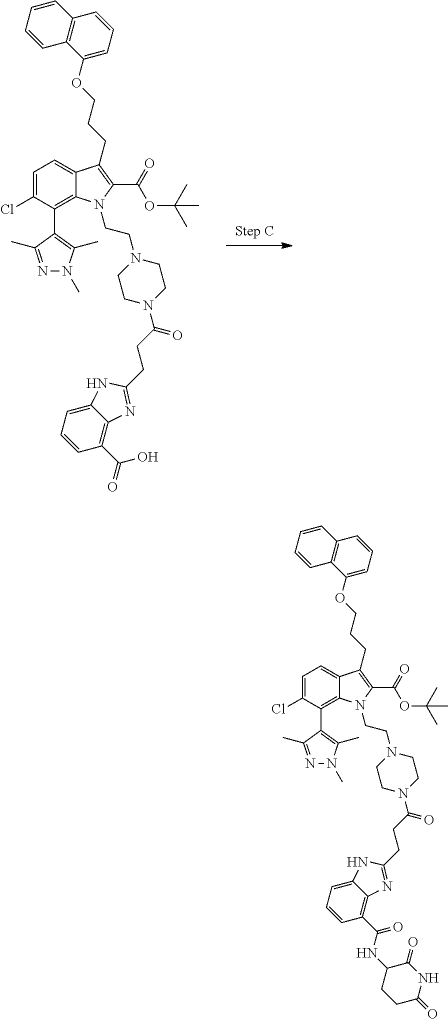 [SMILES: Cc1nn(C)c(C)c1-c1c(Cl)ccc2c(CCCOc3cccc4ccccc34)c(C(=O)OC(C)(C)C)n(CCN3CCN(C(=O)CCc4nc5c(C(=O)O)cccc5[nH]4)CC3)c12 ; Cc1nn(C)c(C)c1-c1c(Cl)ccc2c(CCCOc3cccc4ccccc34)c(C(=O)OC(C)(C)C)n(CCN3CCN(C(=O)CCc4nc5c(C(=O)NC6CCC(=O)NC6=O)cccc5[nH]4)CC3)c12]

To a well stirred solution of 3-aminopiperidine-2,6-dione hydrochloride (7.8 mg, 0.047 mmol) and 2-{3-[4-(2-{2-[(tert-butoxy)carbonyl]-6-chloro-3-[3-(naphthalen-1-yloxy)propyl]-7-(1,3,5-trimethyl-1H-pyrazol-4-yl)-1H-indol-1-yl}ethyl)piperazin-1-yl]-3-oxopropyl}-1H-1,3-benzodiazole-4-carboxylic acid (23.0 mg) in DMF (0.237 mL) was added DIPEA (0.012 mL, 0.071 mmol) and HATU (10.8 mg, 0.028 mmol) and the mixture was allowed to stir under nitrogen for 16 h at room temperature. After complete consumption of the starting material solvents were removed under reduced pressure and the reaction mixture diluted with DCM washed successively with cold water (3 times) and brine. Organic layer was dried over Na₂SO₄, filtered and evaporated under reduced pressure. Crude of tert-butyl 6-chloro-1-{2-[4-(3-{4-[(2,6-dioxopiperidin-3-yl)carbamoyl]-1H-1,3-benzodiazol-2-yl}propanoyl)piperazin-1-yl]ethyl}-3-[3-(naphthalen-1-yloxy)propyl]-7-(1,3,5-trimethyl-1H-pyrazol-4-yl)-1H-indole-2-carboxylate (15.0 mg, crude) as brownish solid was used for next step without further purification.

LCMS (ESI+): m/z 981.6 [M+H]⁺

### Step D

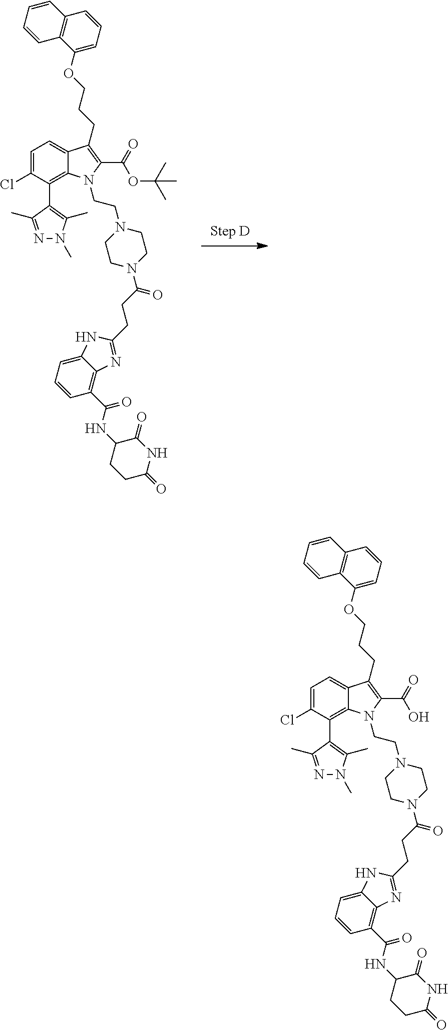 [SMILES: Cc1nn(C)c(C)c1-c1c(Cl)ccc2c(CCCOc3cccc4ccccc34)c(C(=O)OC(C)(C)C)n(CCN3CCN(C(=O)CCc4nc5c(C(=O)NC6CCC(=O)NC6=O)cccc5[nH]4)CC3)c12 ; Cc1nn(C)c(C)c1-c1c(Cl)ccc2c(CCCOc3cccc4ccccc34)c(C(=O)O)n(CCN3CCN(C(=O)CCc4nc5c(C(=O)NC6CCC(=O)NC6=O)cccc5[nH]4)CC3)c12]

Tert-butyl 6-chloro-1-{2-[4-(3-{4-[(2,6-dioxopiperidin-3-yl)carbamoyl]-1H-1,3-benzodiazol-2-yl}propanoyl)piperazin-1-yl]ethyl}-3-[3-(naphthalen-1-yloxy)propyl]-7-(1,3,5-trimethyl-1H-pyrazol-4-yl)-1H-indole-2-carboxylate (15.0 mg, crude) was dissolved in DCM (0.104 mL) and TFA (0.013 mL, 0.175 mmol) was added. Mixture was stirred for 7 days at room temperature. During this time additional amounts of TFA (1 mL) was added. After full conversion was reached solvents were removed under reduced pressure, and purified using preparative HPLC (H₂O:MeCN+0.1% FA). 6-chloro-1-{2-[4-(3-{4-[(2,6-dioxopiperidin-3-yl)carbamoyl]-1H-1,3-benzodiazol-2-yl}propanoyl)piperazin-1-yl]ethyl}-3-[3-(naphthalen-1-yloxy)propyl]-7-(1,3,5-trimethyl-1H-pyrazol-4-yl)-1H-indole-2-carboxylic acid (1.6 mg, 0.002 mmol, 3% over four steps) was obtained as white solid.

LCMS (ESI+): m/z 925.6 [M+H]⁺

¹H NMR (500 MHz, DMSO) δ 12.70 (s, 1H), 10.51 (t, J=6.3 Hz, 1H), 8.24-8.16 (m, 1H), 7.87-7.82 (m, 1H), 7.80 (dd, J=7.6, 1.1 Hz, 1H), 7.76-7.67 (m, 1H), 7.67-7.58 (m, 1H), 7.54-7.46 (m, 2H), 7.46-7.40 (m, 1H), 7.37 (t, J=7.9 Hz, 1H), 7.27 (t, J=7.7 Hz, 1H), 7.22-7.14 (m, 1H), 6.89 (d, J=7.5 Hz, 1H), 4.88-4.77 (m, 1H), 4.35-4.23 (m, 1H), 4.23-4.17 (m, 3H), 3.73 (d, J=5.0 Hz, 3H), 3.46-3.36 (m, 6H), 3.09 (t, J=7.2 Hz, 2H), 3.02-2.89 (m, 1H), 2.86-2.76 (m, 1H), 2.60-2.52 (m, 2H), 2.23-2.17 (m, 2H), 2.14-2.05 (m, 3H), 2.05-1.97 (m, 8H), 1.87 (d, J=3.7 Hz, 3H).

### Example 14: 6-chloro-1-{2-[4-(2-{4-[(2,6-dioxopiperidin-3-yl)carbamoyl]-2-methyl-1H-1,3-benzodiazol-1-yl}acetyl)piperazin-1-yl]ethyl}-3-[3-(naphthalen-1-yloxy)propyl]-7-(1,3,5-trimethyl-1H-pyrazol-4-yl)-1H-indole-2-carboxylic acid (266)

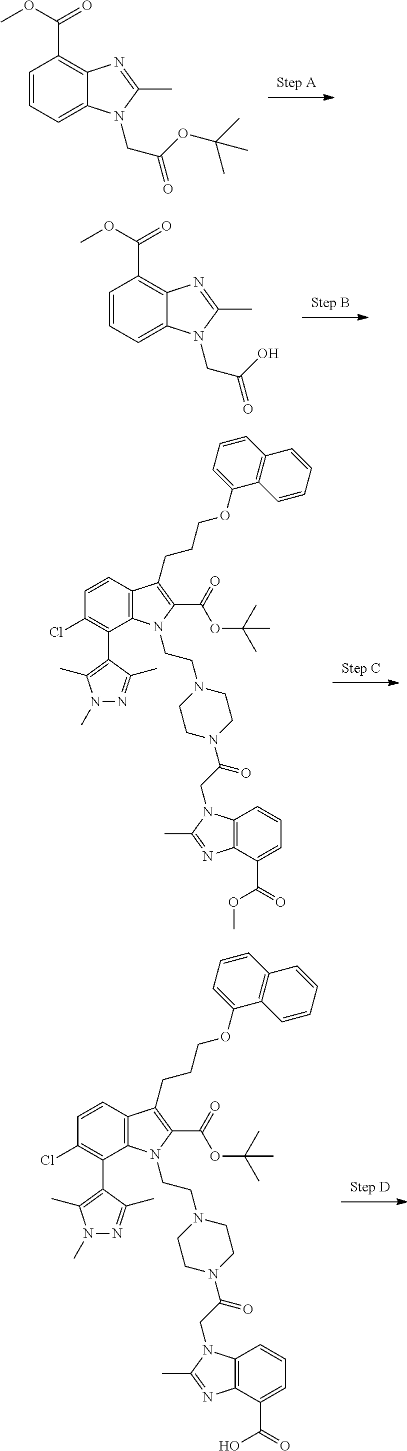

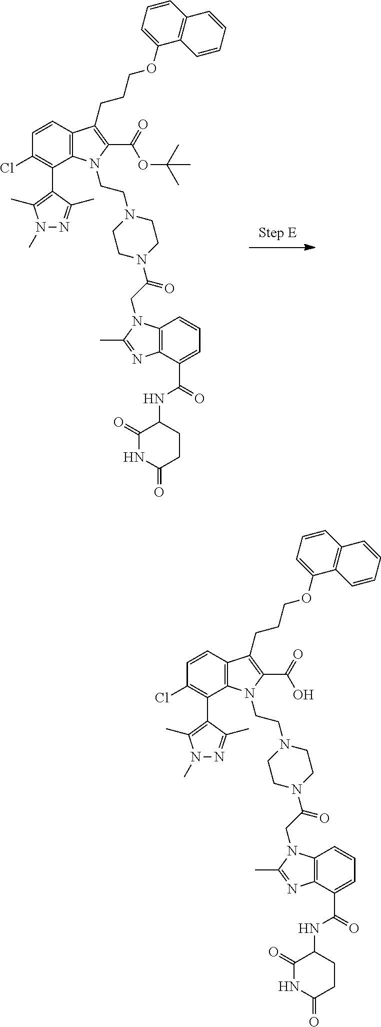 [SMILES: Cc1nn(C)c(C)c1-c1c(Cl)ccc2c(CCCOc3cccc4ccccc34)c(C(=O)OC(C)(C)C)n(CCN3CCN(C(=O)Cn4c(C)nc5c(C(=O)NC6CCC(=O)NC6=O)cccc54)CC3)c12 ; Cc1nn(C)c(C)c1-c1c(Cl)ccc2c(CCCOc3cccc4ccccc34)c(C(=O)O)n(CCN3CCN(C(=O)Cn4c(C)nc5c(C(=O)NC6CCC(=O)NC6=O)cccc54)CC3)c12]

### Step A

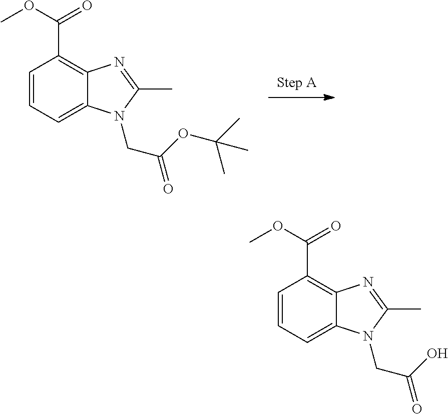 [SMILES: COC(=O)c1cccc2c1nc(C)n2CC(=O)OC(C)(C)C ; COC(=O)c1cccc2c1nc(C)n2CC(=O)O]

Methyl 1-(2-(tert-butoxy)-2-oxoethyl)-2-methyl-1H-benzo[d]imidazole-4-carboxylate (30.4 mg, 0.100 mmol) was dissolved in TFA (3.0 ml) and mixed 16 h at room temperature. Solvent was evaporated under reduced pressure to obtain 24 mg crude of 2-(4-(methoxycarbonyl)-2-methyl-1H-benzo[d]imidazol-1-yl)acetic acid as a brown oil that was used directly in the next step.

LCMS (ESI+): m/z 249.3 [M+H]⁺

### Step B

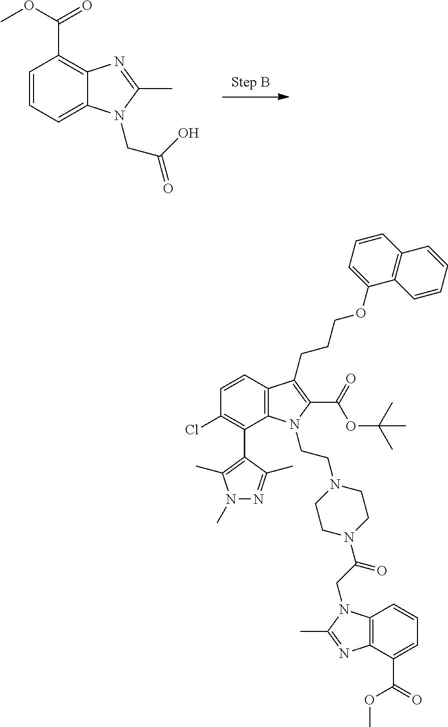 [SMILES: COC(=O)c1cccc2c1nc(C)n2CC(=O)O ; COC(=O)c1cccc2c1nc(C)n2CC(=O)N1CCN(CCn2c(C(=O)OC(C)(C)C)c(CCCOc3cccc4ccccc34)c3ccc(Cl)c(-c4c(C)nn(C)c4C)c32)CC1]

To a well stirred solution of crude 2-(4-(methoxycarbonyl)-2-methyl-1H-benzo[d]imidazol-1-yl)acetic acid (12.0 mg) and tert-butyl 6-chloro-3-(3-(naphthalen-1-yloxy)propyl)-1-(2-(piperazin-1-yl)ethyl)-7-(1,3,5-trimethyl-1H-pyrazol-4-yl)-1H-indole-2-carboxylate (31.1 mg, 0.047 mmol) in DMF (0.474 ml) was added DIPEA (0.023 ml, 0.129 mmol) and HATU (19.8 mg, 0.052 mmol). The mixture was allowed to stir under nitrogen for 16 h at room temperature. After complete consumption of the starting material, the reaction mixture was diluted with EtOAc, washed successively with cold water (3 times) and brine. Organic layer was dried over Na₂SO₄ and evaporated under reduced pressure to afford 41.0 mg of crude methyl 1-(2-(4-(2-(2-(tert-butoxycarbonyl)-6-chloro-3-(3-(naphthalen-1-yloxy)propyl)-7-(1,3,5-trimethyl-1H-pyrazol-4-yl)-1H-indol-1-yl)ethyl)piperazin-1-yl)-2-oxoethyl)-2-methyl-1H-benzo[d]imidazole-4-carboxylate as brown solid which was used for next step without further purification.

LCMS (ESI+): m/z 886.8 [M+H]⁺

### Step C

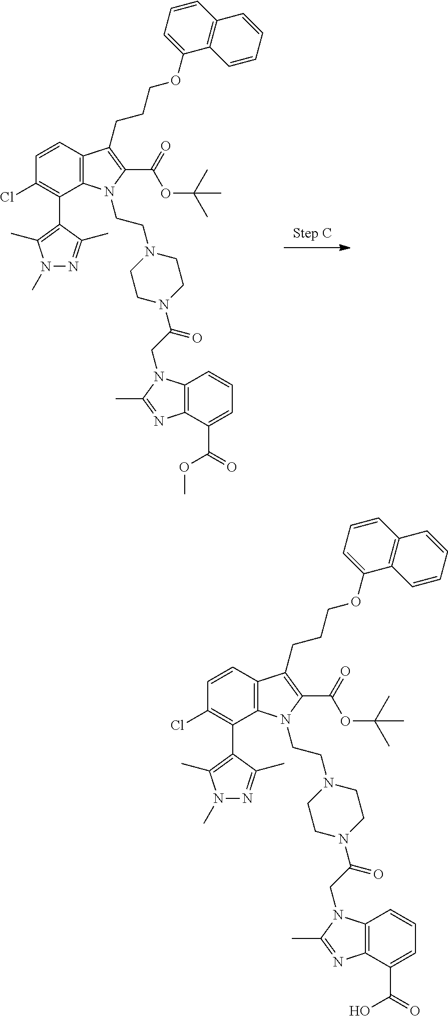 [SMILES: COC(=O)c1cccc2c1nc(C)n2CC(=O)N1CCN(CCn2c(C(=O)OC(C)(C)C)c(CCCOc3cccc4ccccc34)c3ccc(Cl)c(-c4c(C)nn(C)c4C)c32)CC1 ; Cc1nn(C)c(C)c1-c1c(Cl)ccc2c(CCCOc3cccc4ccccc34)c(C(=O)OC(C)(C)C)n(CCN3CCN(C(=O)Cn4c(C)nc5c(C(=O)O)cccc54)CC3)c12]

Methyl 1-(2-(4-(2-(2-(tert-butoxycarbonyl)-6-chloro-3-(3-(naphthalen-1-yloxy)propyl)-7-(1,3,5-trimethyl-1H-pyrazol-4-yl)-1H-indol-1-yl)ethyl)piperazin-1-yl)-2-oxoethyl)-2-methyl-1H-benzo[d]imidazole-4-carboxylate (41.0 mg, crude) was dissolved in MeOH (4.6 ml) and a few drops of DCM were added for solubility. Water (4.6 ml) was added followed by aqueous solution of 1M LiOH (0.231 mL, 0.231 mmol) and the mixture was stirred in room temperature for 16 h. To mixture water and 1M HCl were added till pH=3 and the mixture was extracted with DCM to afford 40 mg of crude 1-(2-(4-(2-(2-(tert-butoxycarbonyl)-6-chloro-3-(3-(naphthalen-1-yloxy)propyl)-7-(1,3,5-trimethyl-1H-pyrazol-4-yl)-1H-indol-1-yl)ethyl)piperazin-1-yl)-2-oxoethyl)-2-methyl-1H-benzo[d]imidazole-4-carboxylic acid as a brow gum, which was used for next step without further purification.

LCMS (ESI+): m/z 872.1 [M+H]⁺

### Step D

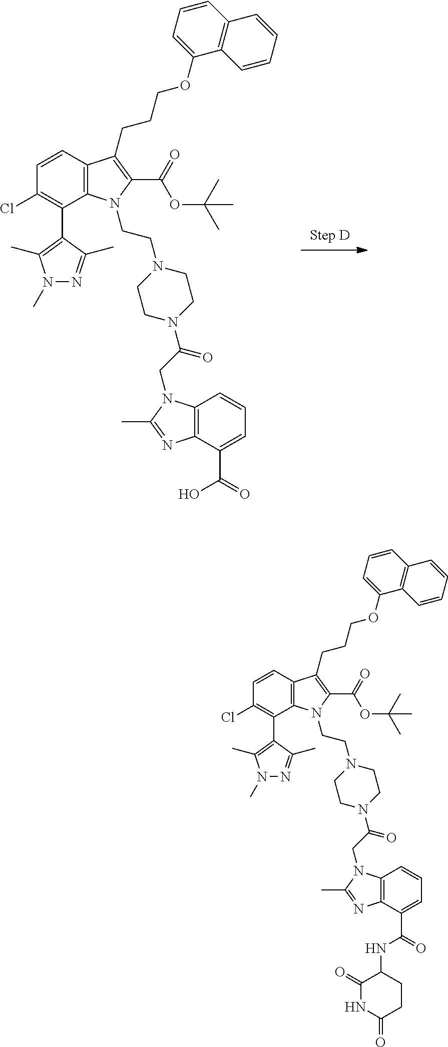 [SMILES: Cc1nn(C)c(C)c1-c1c(Cl)ccc2c(CCCOc3cccc4ccccc34)c(C(=O)OC(C)(C)C)n(CCN3CCN(C(=O)Cn4c(C)nc5c(C(=O)O)cccc54)CC3)c12 ; Cc1nn(C)c(C)c1-c1c(Cl)ccc2c(CCCOc3cccc4ccccc34)c(C(=O)OC(C)(C)C)n(CCN3CCN(C(=O)Cn4c(C)nc5c(C(=O)NC6CCC(=O)NC6=O)cccc54)CC3)c12]

To a well stirred solution of 3-aminopiperidine-2,6-dione hydrochloride (15.1 mg, 0.092 mmol) and 1-(2-(4-(2-(2-(tert-butoxycarbonyl)-6-chloro-3-(3-(naphthalen-1-yloxy)propyl)-7-(1,3,5-trimethyl-1H-pyrazol-4-yl)-1H-indol-1-yl)ethyl)piperazin-1-yl)-2-oxoethyl)-2-methyl-1H-benzo[d]imidazole-4-carboxylic acid (40.0 mg, crude) in DMF (0.458 ml) was added DIPEA (0.024 mL, 0.138 mmol) and HATU (20.9 mg, 0.055 mmol). The mixture was allowed to stir under nitrogen for 16 h. After complete consumption of the starting material, the reaction mixture was diluted with EtOAc, washed successively with cold water (3 times) and brine. Organic layer was dried over Na₂SO₄ and evaporated under reduced pressure to afford 45 mg of crude tert-butyl 6-chloro-1-{2-[4-(2-{4-[(2,6-dioxopiperidin-3-yl)carbamoyl]-2-methyl-1H-1,3-benzodiazol-1-yl}acetyl)piperazin-1-yl]ethyl}-3-[3-(naphthalen-1-yloxy)propyl]-7-(1,3,5-trimethyl-1H-pyrazol-4-yl)-1H-indole-2-carboxylate as a brown solid which was used for next step without further purification.

LCMS (ESI+): m/z 982.0 [M+H]⁺

### Step E

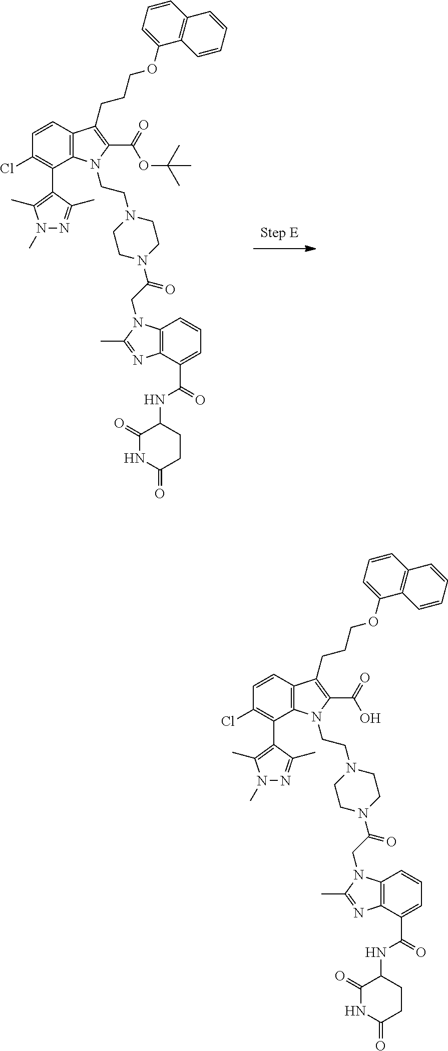 [SMILES: Cc1nn(C)c(C)c1-c1c(Cl)ccc2c(CCCOc3cccc4ccccc34)c(C(=O)OC(C)(C)C)n(CCN3CCN(C(=O)Cn4c(C)nc5c(C(=O)NC6CCC(=O)NC6=O)cccc54)CC3)c12 ; Cc1nn(C)c(C)c1-c1c(Cl)ccc2c(CCCOc3cccc4ccccc34)c(C(=O)O)n(CCN3CCN(C(=O)Cn4c(C)nc5c(C(=O)NC6CCC(=O)NC6=O)cccc54)CC3)c12]

Tert-butyl 6-chloro-1-{2-[4-(2-{4-[(2,6-dioxopiperidin-3-yl)carbamoyl]-2-methyl-1H-1,3-benzodiazol-1-yl}acetyl)piperazin-1-yl]ethyl}-3-[3-(naphthalen-1-yloxy)propyl]-7-(1,3,5-trimethyl-1H-pyrazol-4-yl)-1H-indole-2-carboxylate (45 mg, crude) was suspended in dry DCM (0.342 mL) under argon atmosphere and TFA (0.336 mL, 4.386 mmol) was added. Reaction was stirred in a sealed vial in room temperature. After 16 h full substrate conversion was observed. DCM along with TFA were evaporated under reduced pressure. Crude product was dissolved in DMSO and purified by preparative HPLC (H₂O:MeCN+0.1% FA) to give a corresponding 6-chloro-1-{2-[4-(2-{4-[(2,6-dioxopiperidin-3-yl)carbamoyl]-2-methyl-1H-1,3-benzodiazol-1-yl}acetyl)piperazin-1-yl]ethyl}-3-[3-(naphthalen-1-yloxy)propyl]-7-(1,3,5-trimethyl-1H-pyrazol-4-yl)-1H-indole-2-carboxylic acid (7.0 mg, 0.008 mmol, 17% yield over four steps) as white powder.

LCMS (ESI+): m/z 925.8 [M+H]⁺

¹H NMR (500 MHz, DMSO) δ 13.43 (s, 1H), 10.93 (s, 1H), 10.25 (d, J=7.4 Hz, 1H), 8.24 (dd, J=7.9, 1.8 Hz, 1H), 7.92-7.83 (m, 2H), 7.77 (d, J=8.6 Hz, 1H), 7.66 (dd, J=8.1, 1.1 Hz, 1H), 7.57-7.50 (m, 2H), 7.48 (d, J=8.2 Hz, 1H), 7.41 (t, J=7.9 Hz, 1H), 7.31 (t, J=7.8 Hz, 1H), 7.25 (d, J=8.5 Hz, 1H), 6.93 (d, J=7.6 Hz, 1H), 5.30 (s, 2H), 4.90 (ddd, J=12.6, 7.3, 5.3 Hz, 1H), 4.42-4.28 (m, 1H), 4.28-4.16 (m, 3H), 3.79 (s, 3H), 3.55-3.46 (m, 2H), 3.42-3.36 (m, 3H), 2.84 (ddd, J=17.4, 13.5, 5.6 Hz, 1H), 2.62-2.59 (m, 1H), 2.57-2.55 (m, 1H), 2.49 (s, 3H), 2.32-2.21 (m, 5H), 2.21-2.06 (m, 5H), 2.04 (s, 3H), 1.91 (s, 3H).

### Example 15: 6-chloro-1-(2-{4-[2-({7-[(2,6-dioxopiperidin-3-yl)carbamoyl]-2-(trifluoromethyl)thieno[3,4-b]pyridin-4-yl}oxy)acetyl]piperazin-1-yl}ethyl)-3-[3-(naphthalen-1-yloxy)propyl]-7-(1,3,5-trimethyl-1H-pyrazol-4-yl)-1H-indole-2-carboxylic acid (267)

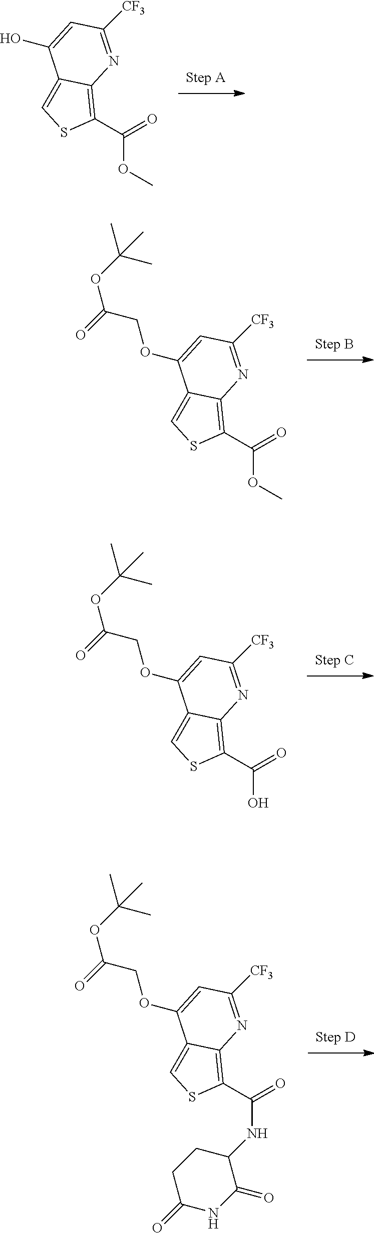 [SMILES: COC(=O)c1scc2c(O)cc(C(F)(F)F)nc12 ; COC(=O)c1scc2c(OCC(=O)OC(C)(C)C)cc(C(F)(F)F)nc12 ; CC(C)(C)OC(=O)COc1cc(C(F)(F)F)nc2c(C(=O)O)scc12 ; CC(C)(C)OC(=O)COc1cc(C(F)(F)F)nc2c(C(=O)NC3CCC(=O)NC3=O)scc12]

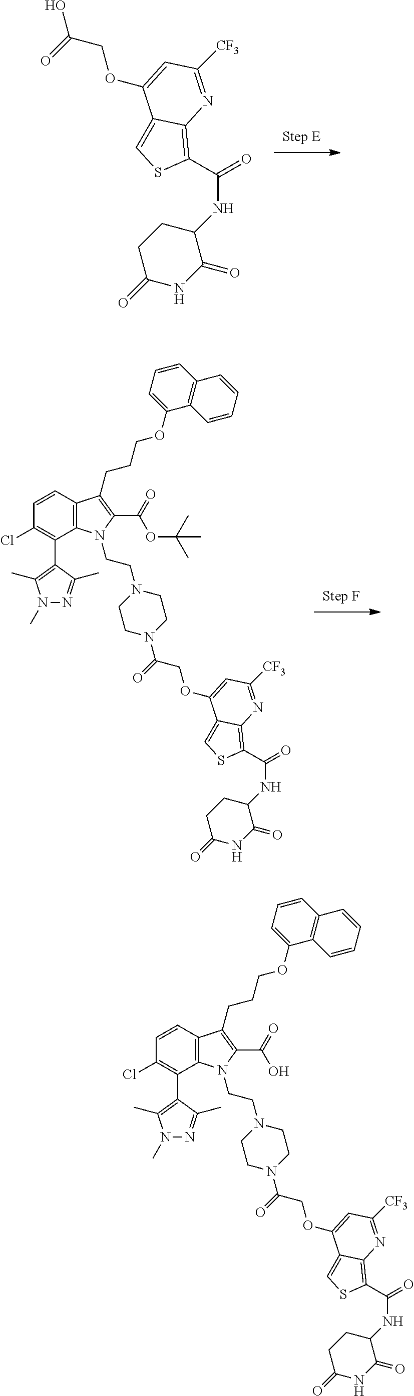 [SMILES: O=C(O)COc1cc(C(F)(F)F)nc2c(C(=O)NC3CCC(=O)NC3=O)scc12 ; Cc1nn(C)c(C)c1-c1c(Cl)ccc2c(CCCOc3cccc4ccccc34)c(C(=O)OC(C)(C)C)n(CCN3CCN(C(=O)COc4cc(C(F)(F)F)nc5c(C(=O)NC6CCC(=O)NC6=O)scc45)CC3)c12 ; Cc1nn(C)c(C)c1-c1c(Cl)ccc2c(CCCOc3cccc4ccccc34)c(C(=O)O)n(CCN3CCN(C(=O)COc4cc(C(F)(F)F)nc5c(C(=O)NC6CCC(=O)NC6=O)scc45)CC3)c12]

### Step A

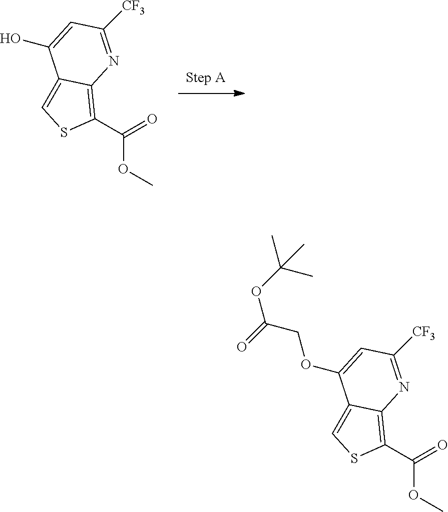 [SMILES: COC(=O)c1scc2c(O)cc(C(F)(F)F)nc12 ; COC(=O)c1scc2c(OCC(=O)OC(C)(C)C)cc(C(F)(F)F)nc12]

Methyl 4-hydroxy-2-(trifluoromethyl)thieno[3,4,b]pyridine-7-carboxylate (200.0 mg, 0.721 mmol) was dissolved in dry DMF (2.0 mL). To the mixture was added tert-butyl bromoacetate (0.160 mL, 1.082 mmol) and K₂CO₃ (199.4 mg, 1.443 mmol). The reaction was stirred for 24 h at RT. The solvent was evaporated and the crude was dissolved in EtOAc and purified by flash chromatography (SiO₂, isohaxane:EtOAc, 0-50%). Isolated fraction was concentrated in vacuo. The product, methyl 4-[2-(tert-butoxy)-2-oxoethoxy]-2-(trifluoromethyl)thieno[3,4-b]pyridine-7-carboxylate (247.0 mg, 0.631 mmol, 87.5%) was a light yellow solid.

LCMS (ESI⁺): m/z 392.0 [M+H]⁺

### Step B

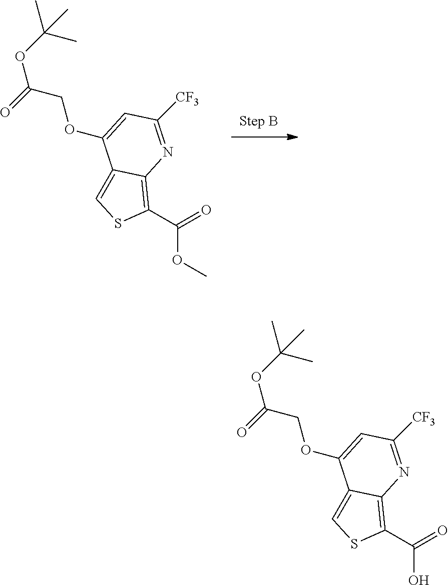 [SMILES: COC(=O)c1scc2c(OCC(=O)OC(C)(C)C)cc(C(F)(F)F)nc12 ; CC(C)(C)OC(=O)COc1cc(C(F)(F)F)nc2c(C(=O)O)scc12]

To a solution of methyl 4-[2-(tert-butoxy)-2-oxoethoxy]-2-(trifluoromethyl)thieno[3,4-b]pyridine-7-carboxylate (247.0 mg, 0.631 mmol) in a mixture of acetonitrile (15.0 mL) and water (1.5 mL) was added LiBr (548.1 mg, 6.311 mmol) and Et₃N (0.494 mL, 3.552 mmol). The mixture was stirred at 60° C. for 2 days and 24 h at RT. The crude was concentrated in vacuo. Residues was dissolved in EtOAc and was extracted with water and brine. Organic layer was dried over Na₂SO₄, filtered and concentrated in vacuo. The product, 4-[2-(tert-butoxy)-2-oxoethoxy]-2-(trifluoromethyl)thieno[3,4-b]pyridine-7-carboxylic acid (233.9 mg, crude) was a yellow solid and was used without further purification to next step.

LCMS (ESI⁺): m/z 378.0 [M+H]⁺

### Step C

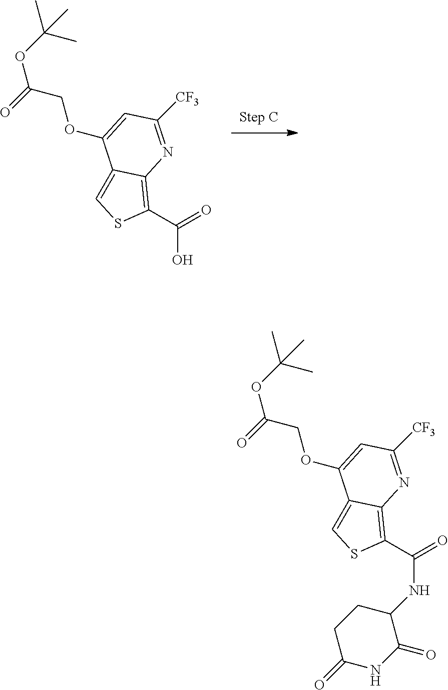 [SMILES: CC(C)(C)OC(=O)COc1cc(C(F)(F)F)nc2c(C(=O)O)scc12 ; CC(C)(C)OC(=O)COc1cc(C(F)(F)F)nc2c(C(=O)NC3CCC(=O)NC3=O)scc12]

4-[2-(Tert-butoxy)-2-oxoethoxy]-2-(trifluoromethyl)thieno[3,4-b]pyridine-7-carboxylic acid (55.0 mg, crude) and 3-aminopiperidine-2,6-dione hydrochloride (28.8 mg, 0.175 mmol) was dissolved in DMF (1.5 mL). To the mixture were added DIPEA (0.076 mL, 0.437 mmol) and HATU (83.1 mg, 0.219 mmol). The reaction was stirred at RT for 18 h. The solvent was evaporated. The residues was dissolved in EtOAc, washed two times with brine, dried over Na₂SO₄, filtered and concentrated in vacuo. The mixture was purified by flash chromatography (SiO₂, DCM:MeOH, 0-20%) to give tert-butyl 2-({7-[(2,6-dioxopiperidin-3-yl)carbamoyl]-2-(trifluoromethyl)thieno[3,4-b]pyridin-4-yl}oxy)acetate (66.0 mg, crude) as a yellow solid.

LCMS (ESI⁺): m/z 487.9 [M+H]⁺

### Step D

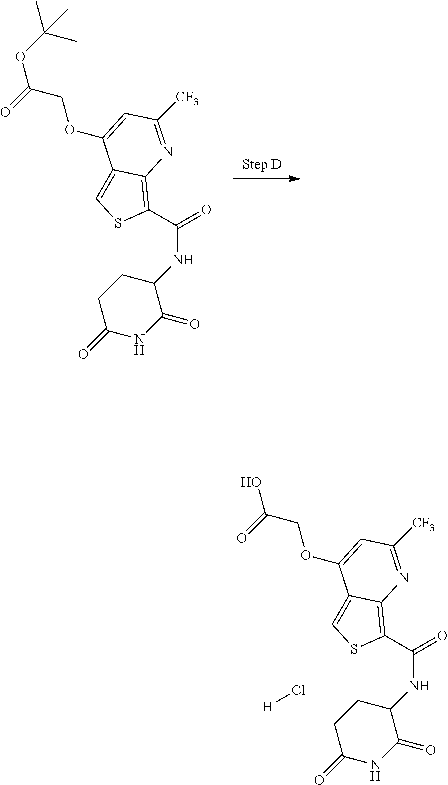 [SMILES: CC(C)(C)OC(=O)COc1cc(C(F)(F)F)nc2c(C(=O)NC3CCC(=O)NC3=O)scc12 ; O=C(O)COc1cc(C(F)(F)F)nc2c(C(=O)NC3CCC(=O)NC3=O)scc12 ; Cl]

To the mixture of tert-butyl 2-({7-[(2,6-dioxopiperidin-3-yl)carbamoyl]-2-(trifluoromethyl)thieno[3,4-b]pyridin-4-yl}oxy)acetate (66.0 mg, crude) in DCM (1 mL) was added TFA (1 mL). The mixture was stirred for 17 h at RT. The solvent was evaporated and to the crude was added HCl and mixture was concentrated in vacuo. The product, 2-({7-[(2,6-dioxopiperidin-3-yl)carbamoyl]-2-(trifluoromethyl)thieno[3,4-b]pyridin-4-yl}oxy)acetic acid hydrochloride (54.0 mg, crude) as yellow solid was used to next step without further purification.

LCMS (ESI⁺): m/z 431.8 [M+H]⁺

### Step E

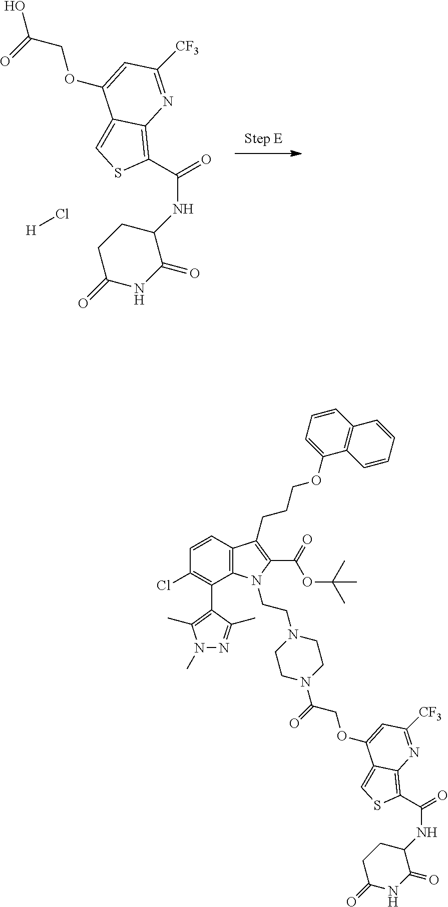 [SMILES: O=C(O)COc1cc(C(F)(F)F)nc2c(C(=O)NC3CCC(=O)NC3=O)scc12 ; Cl ; Cc1nn(C)c(C)c1-c1c(Cl)ccc2c(CCCOc3cccc4ccccc34)c(C(=O)OC(C)(C)C)n(CCN3CCN(C(=O)COc4cc(C(F)(F)F)nc5c(C(=O)NC6CCC(=O)NC6=O)scc45)CC3)c12]

To a solution of 2-({7-[(2,6-dioxopiperidin-3-yl)carbamoyl]-2-(trifluoromethyl)thieno[3,4-b]pyridin-4-yl}oxy)acetic acid hydrochloride (30.0 mg, crude) and tert-butyl 6-chloro-3-[3-(naphthalen-1-yloxy)propyl]-1-[2-(piperazin-1-yl)ethyl]-7-(1,3,5-trimethyl-1H-pyrazol-4-yl)-1H-indole-2-carboxylate (41.5 mg, 0.063 mmol) in dry DMF (2.0 mL) was added DIPEA (0.055 mL, 0.316 mmol) and HATU (48.1 mg, 0.126 mmol). The mixture was stirred for 2 h at RT. Then, the crude was concentrated in vacuo. The residues were dissolved in DCM and washed with H₂O and brine. The organic layers was dried over Na₂SO₄, filtered and evaporated under reduced pressure. Tert-butyl 6-chloro-1-(2-{4-[2-({7-[(2,6-dioxopiperidin-3-yl)carbamoyl]-2-(trifluoromethyl)thieno[3,4-b]pyridin-4-yl}oxy)acetyl]piperazin-1-yl}ethyl)-3-[3-(naphthalen-1-yloxy)propyl]-7-(1,3,5-trimethyl-1H-pyrazol-4-yl)-1H-indole-2-carboxylate (35.0 mg, crude) was used to next step without further purification.

LCMS (ESI⁺): m/z 1068.9 [M+H]⁺

### Step F

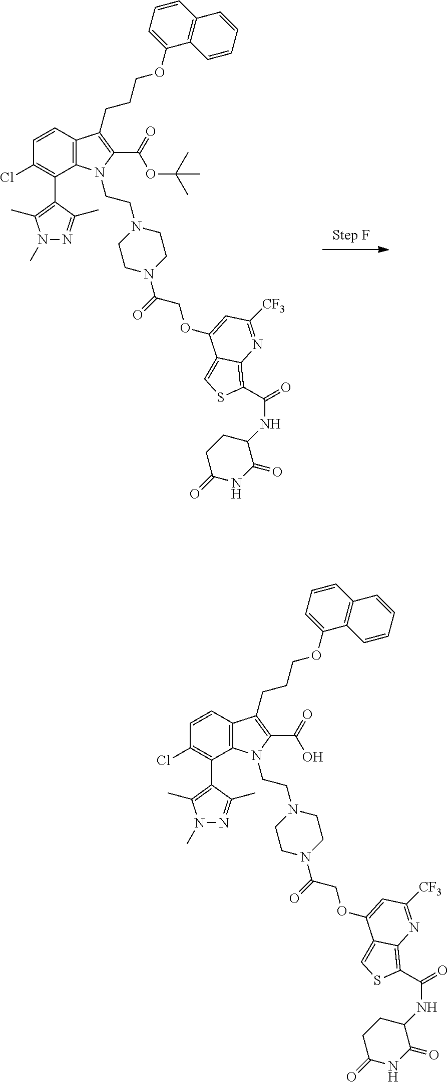 [SMILES: Cc1nn(C)c(C)c1-c1c(Cl)ccc2c(CCCOc3cccc4ccccc34)c(C(=O)OC(C)(C)C)n(CCN3CCN(C(=O)COc4cc(C(F)(F)F)nc5c(C(=O)NC6CCC(=O)NC6=O)scc45)CC3)c12 ; Cc1nn(C)c(C)c1-c1c(Cl)ccc2c(CCCOc3cccc4ccccc34)c(C(=O)O)n(CCN3CCN(C(=O)COc4cc(C(F)(F)F)nc5c(C(=O)NC6CCC(=O)NC6=O)scc45)CC3)c12]

To a solution of tert-butyl 6-chloro-1-(2-{4-[2-({7-[(2,6-dioxopiperidin-3-yl)carbamoyl]-2-(trifluoromethyl)thieno[3,4-b]pyridin-4-yl}oxy)acetyl]piperazin-1-yl}ethyl)-3-[3-(naphthalen-1-yloxy)propyl]-7-(1,3,5-trimethyl-1H-pyrazol-4-yl)-1H-indole-2-carboxylate (35.0 mg, crude) in DCM (1.0 mL) was added TFA (1.0 mL, 13.059 mmol). The reaction was stirred for 17 h at RT. The crude was concentrated in vacuo, dissolved in DMSO and purified by preparative HPLC (H₂O:MeCN+0.1% FA) to give 6-chloro-1-(2-{4-[2-({7-[(2,6-dioxopiperidin-3-yl)carbamoyl]-2-(trifluoromethyl)thieno[3,4-b]pyridin-4-yl}oxy)acetyl]piperazin-1-yl}ethyl)-3-[3-(naphthalen-1-yloxy)propyl]-7-(1,3,5-trimethyl-1H-pyrazol-4-yl)-1H-indole-2-carboxylic acid (10.6 mg, 0.010 mmol, 32.0%) as a light yellow solid.

LCMS (ESI⁺): m/z 1012.69 [M+H]⁺

¹H NMR (500 MHz, DMSO) δ 13.38 (s, 1H), 10.99 (s, 1H), 9.43 (d, J=6.5 Hz, 1H), 8.71 (s, 1H), 8.24-8.17 (m, 1H), 7.85 (dd, J=7.3, 2.0 Hz, 1H), 7.75 (d, J=8.6 Hz, 1H), 7.50 (dtd, J=8.0, 6.8, 5.2 Hz, 2H), 7.45 (d, J=8.3 Hz, 1H), 7.38 (t, J=7.9 Hz, 1H), 7.22 (d, J=8.6 Hz, 1H), 7.05 (s, 1H), 6.93-6.88 (m, 1H), 5.30 (s, 2H), 4.87 (ddd, J=12.2, 6.6, 5.2 Hz, 1H), 4.35-4.26 (m, 1H), 4.22 (t, J=6.1 Hz, 3H), 3.76 (s, 3H), 3.37 (d, J=12.9 Hz, 2H), 3.27 (d, J=10.3 Hz, 2H), 2.87-2.77 (m, 1H), 2.52-2.62 (m, 3H), 2.25-2.18 (m, 2H), 2.18-2.00 (m, 11H), 1.88 (s, 3H).

### Example 16: 6-chloro-1-{2-[4-(2-{(2-(2,6-dioxopiperidin-3-yl)-1,3-dioxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (214)

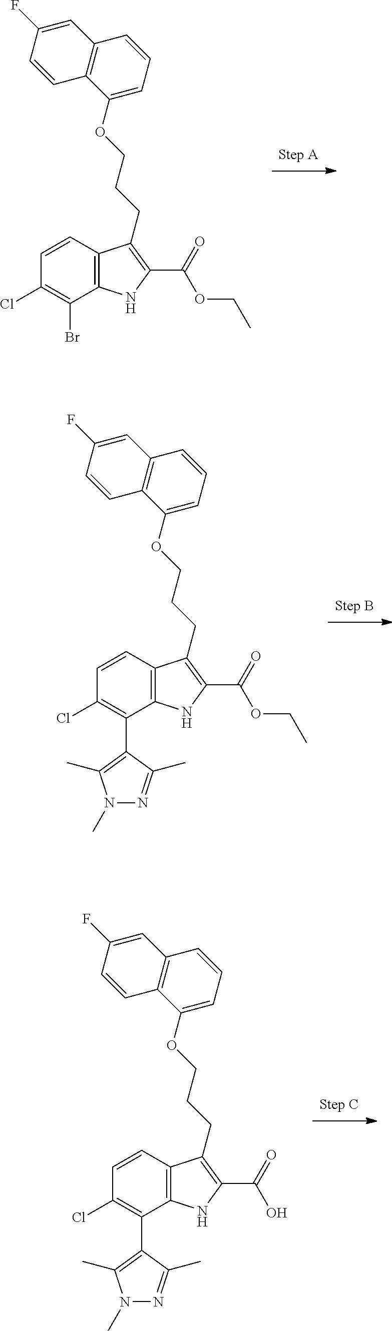

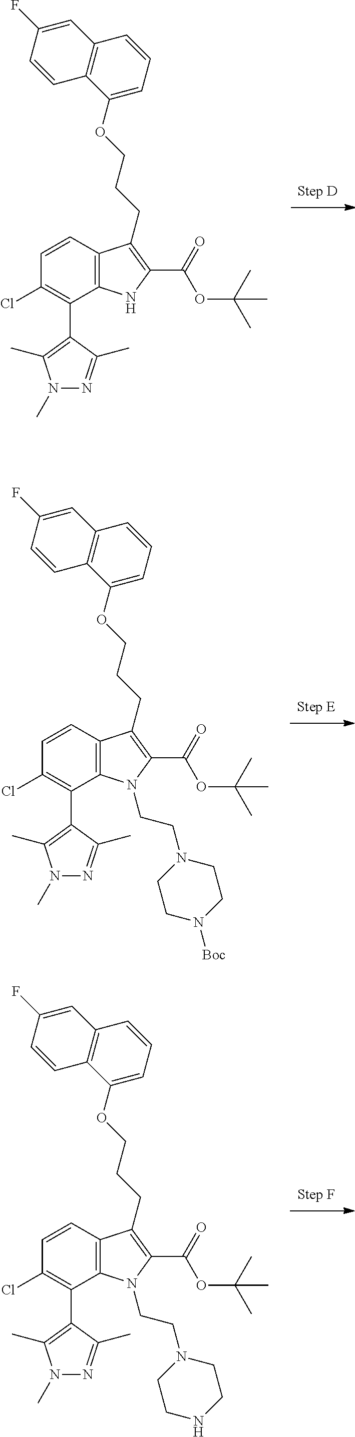

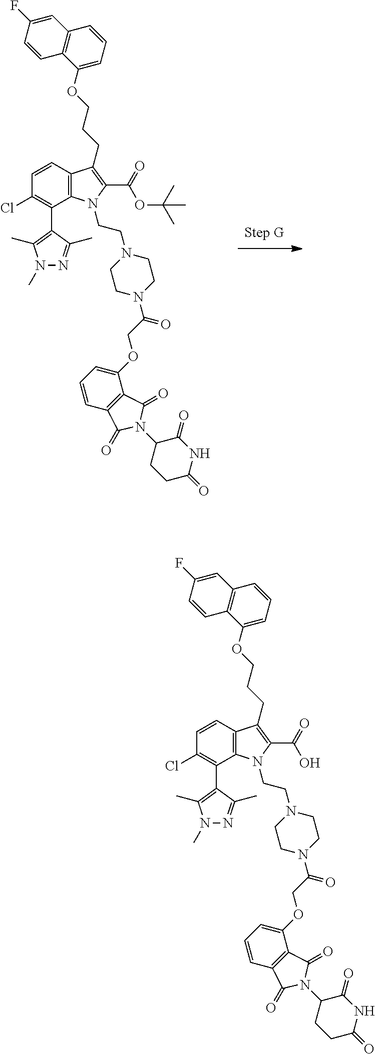 [SMILES: Cc1nn(C)c(C)c1-c1c(Cl)ccc2c(CCCOc3cccc4cc(F)ccc34)c(C(=O)OC(C)(C)C)n(CCN3CCN(C(=O)COc4cccc5c4C(=O)N(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4cccc5c4C(=O)N(C4CCC(=O)NC4=O)C5=O)CC3)c12]

### Step A

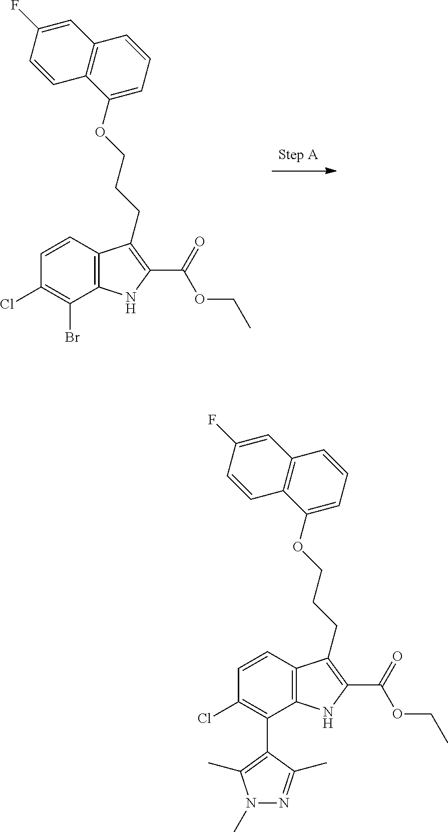 [SMILES: CCOC(=O)c1[nH]c2c(Br)c(Cl)ccc2c1CCCOc1cccc2cc(F)ccc12 ; CCOC(=O)c1[nH]c2c(-c3c(C)nn(C)c3C)c(Cl)ccc2c1CCCOc1cccc2cc(F)ccc12]

To a stirred solution of ethyl 7-bromo-6-chloro-3-(3-((6-fluoronaphthalen-1-yl)oxy)propyl)-1H-indole-2-carboxylate (1.7 g, 3.368 mmol) in dioxane (20 mL) and water (5 mL) were added 1,3,5-trimethyl-4-(4,4,5,5-tetramethyl-1,3,2-dioxaborolan-2-yl)-1H-pyrazole (2.386 g, 10.103 mmol) and K₂CO₃ (1.859 g, 13.471 mmol). The mixture was deoxygenated with argon and to it was added Pd(dppf)Cl₂ (0.369 g, 0.505 mmol) under argon atmosphere. Then the reaction mixture was heated under reflux for 16 h. After complete consumption of the starting material (monitored by TLC and LCMS) the reaction mixture was filtered through celite pad and the solvents were evaporated under reduced pressure get the crude material. It was then diluted with EtOAc, washed successively with water and brine, the organic layer was dried over Na₂SO₄ and evaporated under reduced pressure to get the crude compound, which was then purified by column chromatography (SiO₂, 50% EtOAc in hexane) to get ethyl 6-chloro-3-(3-((6-fluoronaphthalen-1-yl)oxy)propyl)-7-(1,3,5-trimethyl-1H-pyrazol-4-yl)-1H-indole-2-carboxylate (1.2 g, 2.247 mmol, 66.72%) as brown solid.

LCMS (ESI+): m/z 534.2 [M+H]⁺

### Step B

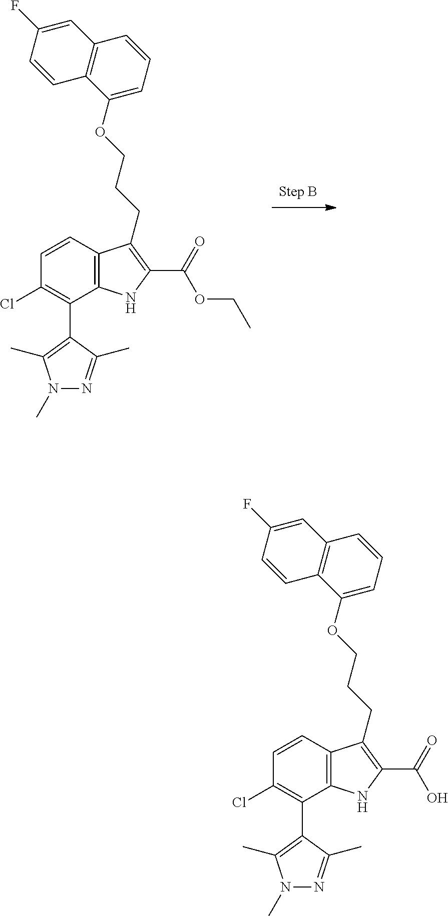 [SMILES: CCOC(=O)c1[nH]c2c(-c3c(C)nn(C)c3C)c(Cl)ccc2c1CCCOc1cccc2cc(F)ccc12 ; Cc1nn(C)c(C)c1-c1c(Cl)ccc2c(CCCOc3cccc4cc(F)ccc34)c(C(=O)O)[nH]c12]

Ethyl 6-chloro-3-(3-((6-fluoronaphthalen-1-yl)oxy)propyl)-7-(1,3,5-trimethyl-1H-pyrazol-4-yl)-1H-indole-2-carboxylate (1.2 g, 2.251 mmol) was dissolved in EtOH (20 mL) and solution of NaOH (0.315 g, 7.88 mmol) in water (10 mL) was added to it. The mixture was heated under reflux for 16 h. After complete consumption of the starting material (monitored by TLC and LCMS) the reaction mixture was cooled down to room temperature, solvents were evaporated under reduced pressure to get the crude reaction mixture. It was then diluted with water, washed with EtOAc. Aqueous layer was carefully acidified using 1M HCl to pH=3 and extracted with EtOAc (×3). The organic layer was dried over Na₂SO₄, filtered, and concentrated in vacuo to afford 6-chloro-3-(3-((6-fluoronaphthalen-1-yl)oxy)propyl)-7-(1,3,5-trimethyl-1H-pyrazol-4-yl)-1H-indole-2-carboxylic acid (0.9 g, crude) as brown liquid which was used for the next step without further purification.

LCMS (ESI+): m/z 506.3 [M+H]⁺

### Step C

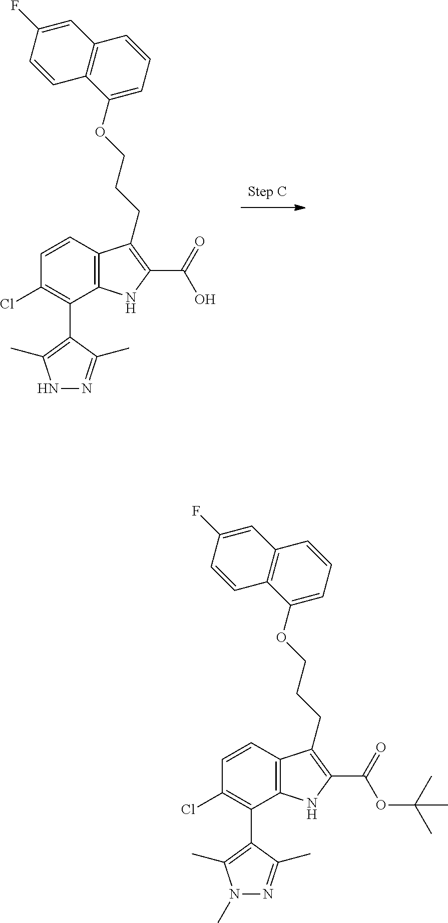 [SMILES: Cc1n[nH]c(C)c1-c1c(Cl)ccc2c(CCCOc3cccc4cc(F)ccc34)c(C(=O)O)[nH]c12 ; Cc1nn(C)c(C)c1-c1c(Cl)ccc2c(CCCOc3cccc4cc(F)ccc34)c(C(=O)OC(C)(C)C)[nH]c12]

6-chloro-3-(3-((6-fluoronaphthalen-1-yl)oxy)propyl)-7-(1,3,5-trimethyl-1H-pyrazol-4-yl)-1H-indole-2-carboxylic acid (1.2 g, 2.376 mmol) was suspended in toluene (20 mL) and the mixture was heated to reflux under nitrogen. N,N-dimethylformamide di-tert-butyl acetal (4.547 mL, 19.01 mmol) was added drop-wise to the refluxing mixture. The mixture was heated under reflux for 16 h under nitrogen. After complete consumption of the starting material (monitored by TLC and LCMS) the reaction mixture was then diluted with EtOAc, washed successively with sodium bicarbonate (aqueous, saturated), water and brine. Organic layer was dried over Na₂SO₄ and evaporated under reduced pressure to get the crude compound, which was then purified by column chromatography (SiO₂, 70% EtOAc in hexane) to get tert-butyl 6-chloro-3-(3-((6-fluoronaphthalen-1-yl)oxy)propyl)-7-(1,3,5-trimethyl-1H-pyrazol-4-yl)-1H-indole-2-carboxylate (0.772 g, 1.373 mmol, 61% over two steps) as brownish liquid.

LCMS (ESI+): m/z 561.9 [M+H]⁺

### Step D

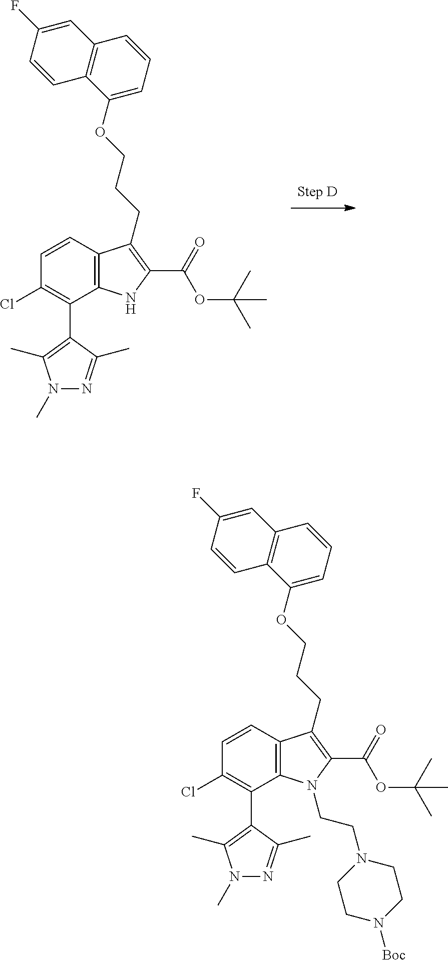 [SMILES: Cc1nn(C)c(C)c1-c1c(Cl)ccc2c(CCCOc3cccc4cc(F)ccc34)c(C(=O)OC(C)(C)C)[nH]c12 ; Cc1nn(C)c(C)c1-c1c(Cl)ccc2c(CCCOc3cccc4cc(F)ccc34)c(C(=O)OC(C)(C)C)n(CCN3CCN(C(=O)OC(C)(C)C)CC3)c12]

To solution of tert-butyl 6-chloro-3-(3-((6-fluoronaphthalen-1-yl)oxy)propyl)-7-(1,3,5-trimethyl-1H-pyrazol-4-yl)-1H-indole-2-carboxylate (0.555 g, 0.989 mmol) in DMF (10 mL) was added tert-butyl 4-(2-chloroethyl)piperazine-1-carboxylate (0.492 g, 1.979 mmol) followed by cesium carbonate (1.607 g, 4.947 mmol) in DMF (5 mL) and the mixture was allowed to stir at 90° C. for 16 h under nitrogen. After complete consumption of the starting material (monitored by TLC and LCMS) the reaction mixture was diluted with EtOAc, washed successively with water and brine. The organic layer was dried over Na₂SO₄ and evaporated under reduced pressure to get the crude compound, which was then purified by column chromatography (SiO₂, 30% EtOAc in hexane) to get tert-butyl 1-(2-(4-(tert-butoxycarbonyl)piperazin-1-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate (0.5 g, 0.645 mmol, 65.27%) as off white solid.

LCMS (ESI+): m/z 774.6 [M+H]⁺

### Step E

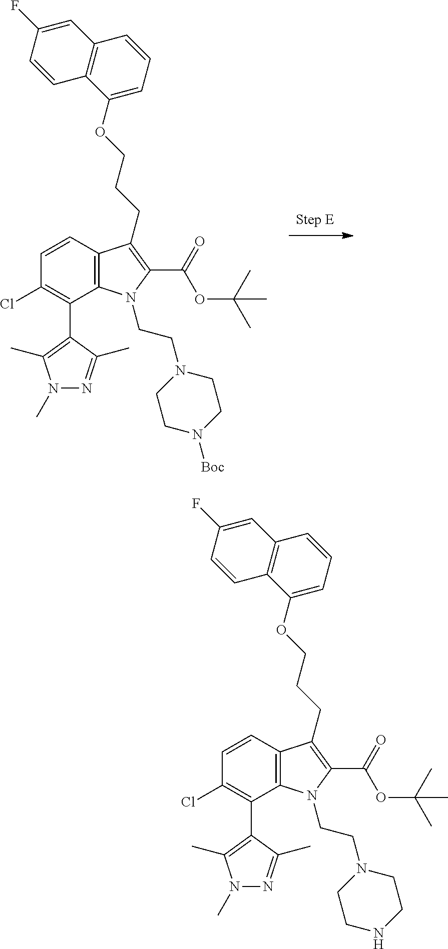

tert-butyl 1-(2-(4-(tert-butoxycarbonyl)piperazin-1-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate (0.3 g, 0.388 mmol) dissolved in 20 mL of 4M HCl in dioxan at 0° C. and the mixture was stirred for 2 h under nitrogen at the same temperature. After complete consumption of the starting material (monitored by TLC and LCMS) the reaction mixture was poured in to cold 1M NaOH solution and extracted several times with DCM. The combined organics were dried over Na₂SO₄ and concentrated in vacuo to get the crude compound which was then purified by column chromatography (amine SiO₂, 10% MeOH in DCM) to get tert-butyl 6-chloro-3-(3-((6-fluoronaphthalen-1-yl)oxy)propyl)-1-(2-(piperazin-1-yl)ethyl)-7-(1,3,5-trimethyl-1H-pyrazol-4-yl)-1H-indole-2-carboxylate (0.165 g, 0.244 mmol, 62.98%) as off white solid.

LCMS (ESI+): m/z 674.4 [M+H]⁺

### Step F

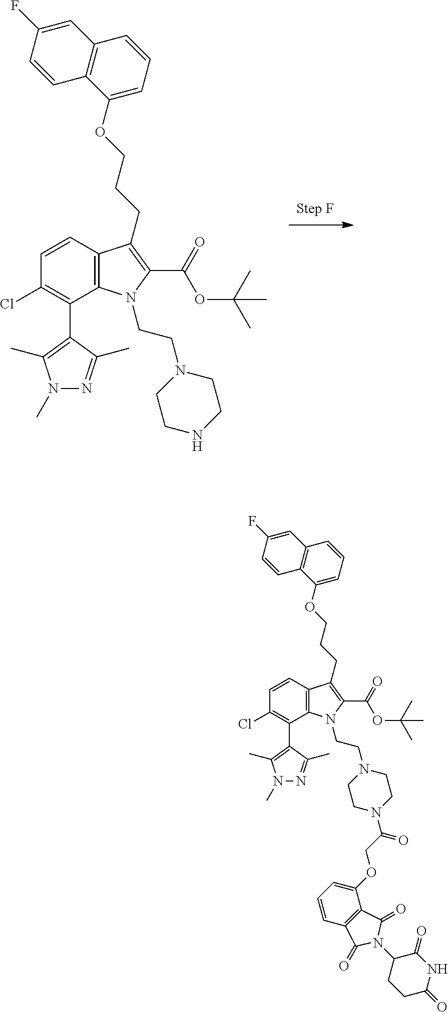 [SMILES: Cc1nn(C)c(C)c1-c1c(Cl)ccc2c(CCCOc3cccc4cc(F)ccc34)c(C(=O)OC(C)(C)C)n(CCN3CCNCC3)c12 ; Cc1nn(C)c(C)c1-c1c(Cl)ccc2c(CCCOc3cccc4cc(F)ccc34)c(C(=O)OC(C)(C)C)n(CCN3CCN(C(=O)COc4cccc5c4C(=O)N(C4CCC(=O)NC4=O)C5=O)CC3)c12]

To a well stirred solution of 2-((2-(2,6-dioxopiperidin-3-yl)-1,3-dioxoisoindolin-4-yl)oxy)acetic acid (18.9 mg, 0.057 mmol) and tert-butyl 6-chloro-3-(3-((6-fluoronaphthalen-1-yl)oxy)propyl)-1-(2-(piperazin-1-yl)ethyl)-7-(1,3,5-trimethyl-1H-pyrazol-4-yl)-1H-indole-2-carboxylate) (32.0 mg, 0.047 mmol) in DMF (0.475 mL) was added DIPEA (0.025 mL, 0.142 mmol) and HATU (21.7 mg, 0.057 mmol) and the mixture was allowed to stir under nitrogen for 2 h. After complete consumption of the starting material (monitored by TLC and LCMS), the reaction mixture diluted with EtOAc, washed successively with cold water (3 times) and brine. Organic layer was dried over Na₂SO₄ and evaporated under reduced pressure. Tert-butyl 6-chloro-1-{2-[4-(2-{[2-(2,6-dioxopiperidin-3-yl)-1,3-dioxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (39.0 mg, crude) was obtained as brown gum, which was used for the next step without further purification.

LCMS (ESI+): m/z 988.2 [M+H]⁺

### Step G

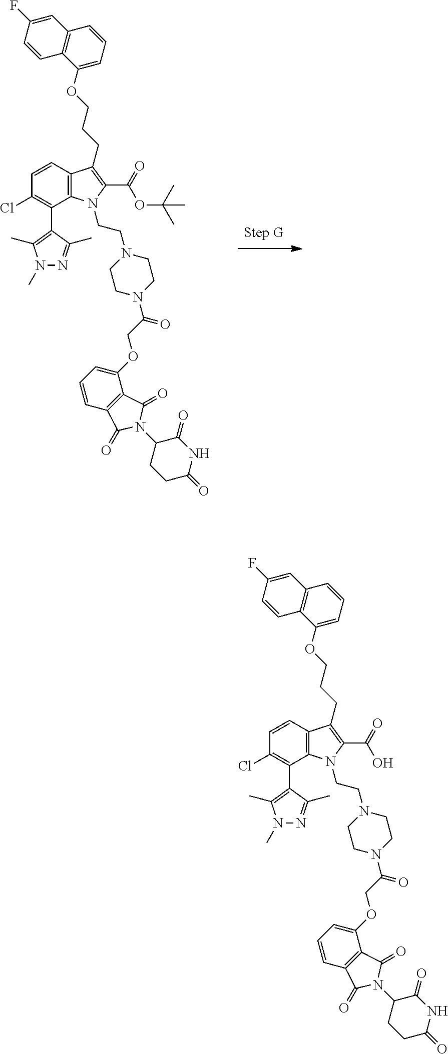 [SMILES: Cc1nn(C)c(C)c1-c1c(Cl)ccc2c(CCCOc3cccc4cc(F)ccc34)c(C(=O)OC(C)(C)C)n(CCN3CCN(C(=O)COc4cccc5c4C(=O)N(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4cccc5c4C(=O)N(C4CCC(=O)NC4=O)C5=O)CC3)c12]

Crude mixture of tert-butyl 6-chloro-1-{2-[4-(2-{[2-(2,6-dioxopiperidin-3-yl)-1,3-dioxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (39.0 mg) was placed in a capped vial, dissolved in DCM (0.28 mL) and then TFA (0.030 mL) was added. Reaction was stirred overnight at RT. Solvents were removed under reduced pressure, and crude was purified using preparative HPLC (H₂O:MeCN+0.1% FA) followed by preparative TLC to afford 6-chloro-1-{2-[4-(2-{[2-(2,6-dioxopiperidin-3-yl)-1,3-dioxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (11.8 mg, 0.013 mmol, 28% over two steps).

LCMS (ESI+): m/z 932.12 [M+H]⁺

¹H NMR (500 MHz, DMSO) δ 13.46 (bs, 1H), 11.11 (s, 1H), 8.26 (dd, J=9.2, 5.9 Hz, 1H), 7.77 (dd, J=8.6, 7.2 Hz, 1H), 7.72 (d, J=8.5 Hz, 1H), 7.67 (dd, J=10.4, 2.6 Hz, 1H), 7.45 (q, J=3.1, 2.3 Hz, 3H), 7.38 (td, J=8.9, 2.7 Hz, 1H), 7.31 (d, J=8.6 Hz, 1H), 7.22 (d, J=8.5 Hz, 1H), 6.90 (dd, J=5.6, 3.1 Hz, 1H), 5.16-5.08 (m, 3H), 4.38-4.28 (m, 1H), 4.26-4.14 (m, 3H), 3.77 (s, 3H), 3.31-3.21 (m, 4H), 2.97-2.84 (m, 1H), 2.64-2.54 (m, 2H), 2.27-2.19 (m, 2H), 2.18-2.00 (m, 12H), 1.89 (s, 3H).

### Example 17: 6-chloro-1-{2-[4-(2-{[2-(2,6-dioxopiperidin-3-yl)-1,3-dioxo-2,3-dihydro-1H-isoindol-4-yl]oxy}acetyl)piperazin-1-yl]ethyl}-3-[3-(5,6,7,8-tetrahydronaphthalen-1-yloxy)propyl]-7-(1,3,5-trimethyl-1H-pyrazol-4-yl)-1H-indole-2-carboxylic acid (216)

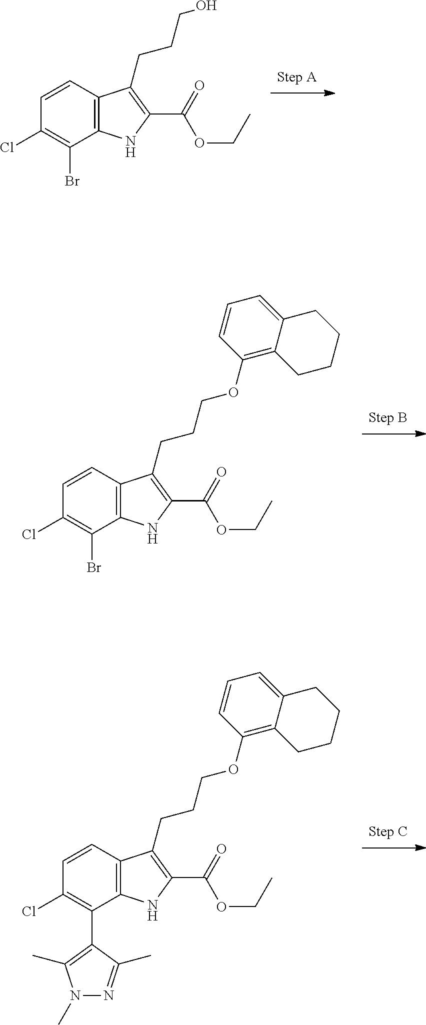

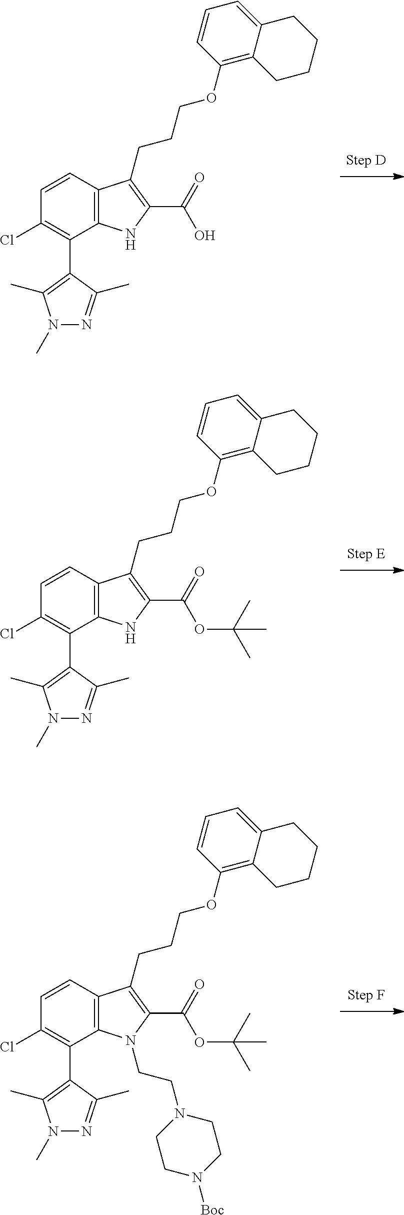 [SMILES: Cc1nn(C)c(C)c1-c1c(Cl)ccc2c(CCCOc3cccc4c3CCCC4)c(C(=O)O)[nH]c12 ; Cc1nn(C)c(C)c1-c1c(Cl)ccc2c(CCCOc3cccc4c3CCCC4)c(C(=O)OC(C)(C)C)[nH]c12 ; Cc1nn(C)c(C)c1-c1c(Cl)ccc2c(CCCOc3cccc4c3CCCC4)c(C(=O)OC(C)(C)C)n(CCN3CCN(C(=O)OC(C)(C)C)CC3)c12]

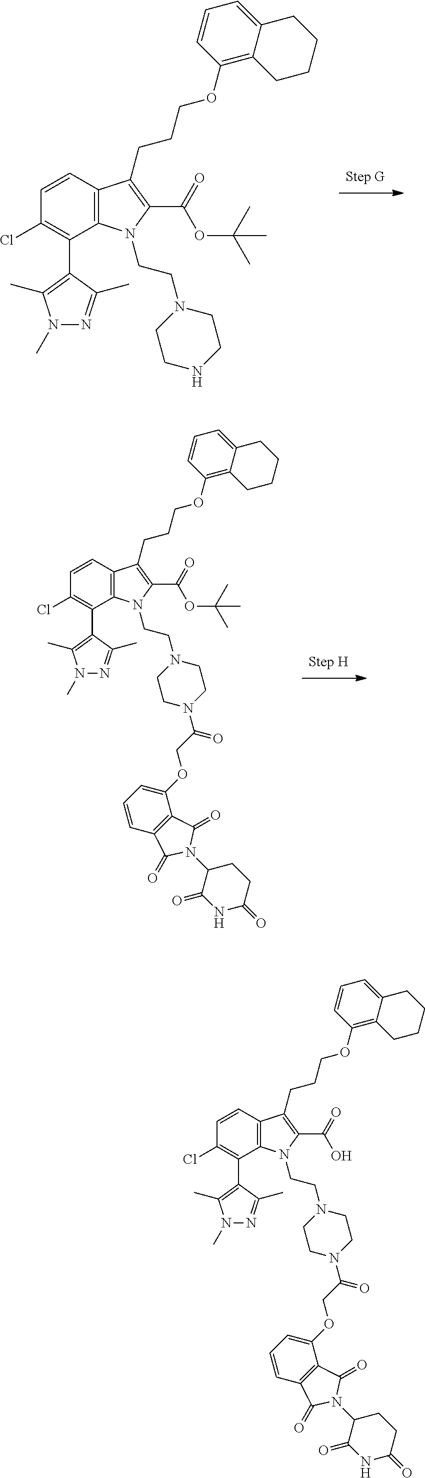 [SMILES: Cc1nn(C)c(C)c1-c1c(Cl)ccc2c(CCCOc3cccc4c3CCCC4)c(C(=O)OC(C)(C)C)n(CCN3CCNCC3)c12 ; Cc1nn(C)c(C)c1-c1c(Cl)ccc2c(CCCOc3cccc4c3CCCC4)c(C(=O)OC(C)(C)C)n(CCN3CCN(C(=O)COc4cccc5c4C(=O)N(C4CCC(=O)NC4=O)C5=O)CC3)c12 ; Cc1nn(C)c(C)c1-c1c(Cl)ccc2c(CCCOc3cccc4c3CCCC4)c(C(=O)O)n(CCN3CCN(C(=O)COc4cccc5c4C(=O)N(C4CCC(=O)NC4=O)C5=O)CC3)c12]

### Step A

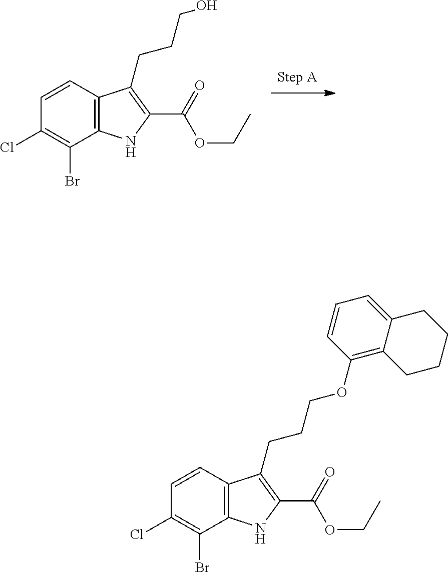 [SMILES: CCOC(=O)c1[nH]c2c(Br)c(Cl)ccc2c1CCCO ; CCOC(=O)c1[nH]c2c(Br)c(Cl)ccc2c1CCCOc1cccc2c1CCCC2]

To a well stirred solution of ethyl 7-bromo-6-chloro-3-(3-hydroxypropyl)-1H-indole-2-carboxylate (5 g, 13.9 mmol) in toluene (65 mL) were added PPh₃ (4.7 g, 18.1 mmol), 5,6,7,8-tetahydronaphthalen-1-ol (4.1 g, 27.7 mmol) and DBAD (6.4 g, 27.9 mmol) consecutively and the reaction mixture was allowed to stir at 130° C. for 16 h under nitrogen. After complete consumption of the starting material, the solvent of reaction mixture was then evaporated under reduced pressure to get the crude compound, which was then purified by flash chromatography (SiO₂, 5% EtOAC in hexane) to get 5 g (10.2 mmol, 73%) of ethyl 7-bromo-6-chloro-3-(3-((5,6,7,8-tetrahydronaphthalen-1-yl)oxy)propyl)-1H-indole-2-carboxylate as white solid.

### Step B

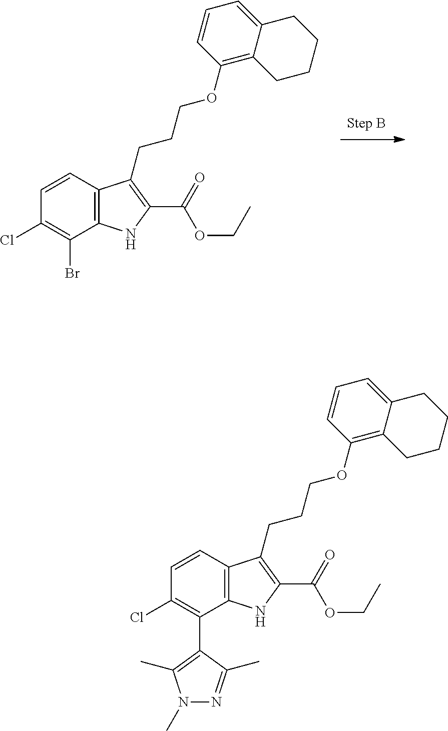 [SMILES: CCOC(=O)c1[nH]c2c(Br)c(Cl)ccc2c1CCCOc1cccc2c1CCCC2 ; CCOC(=O)c1[nH]c2c(-c3c(C)nn(C)c3C)c(Cl)ccc2c1CCCOc1cccc2c1CCCC2]

To a stirred solution of ethyl 7-bromo-6-chloro-3-(3-((5,6,7,8-tetrahydronaphthalen-1-yl)oxy)propyl)-1H-indole-2-carboxylate (5.0 g, 10.2 mmol) in dioxane (50 ml) and water (10 ml) were added 1,3,5-trimethyl-4-(4,4,5,5-tetramethyl-1,3,2-dioxaborolan-2-yl)-1H-pyrazole (7.2 g, 30.7 mmol) and K₂CO₃ (5.6 g, 40.9 mmol). The mixture was deoxygenated with argon and to it was added Pd(dppf)Cl₂ (1.12 g, 1.5 mmol) under argon atmosphere. Then the reaction mixture was heated under reflux for 16 h. After complete consumption of the starting material, the reaction mixture was filtered through celite pad and the solvents were evaporated under reduced pressure get the crude material. It was then diluted with EtOAc, washed successively with water and brine, the organic layer was dried over Na₂SO₄ and evaporated under reduced pressure to get the crude compound, which was then purified by flash chromatography (SiO₂, 50% EtOAc in hexane) to get 4 g (7.7 mmol, 75%) of ethyl 6-chloro-3-(3-((5,6,7,8-tetrahydronaphthalen-1-yl)oxy)propyl)-7-(1,3,5-trimethyl-1H-pyrazol-4-yl)-1H-indole-2-carboxylate as brown solid.

LCMS (ESI+): m/z 520.2 [M+H]⁺

### Step C

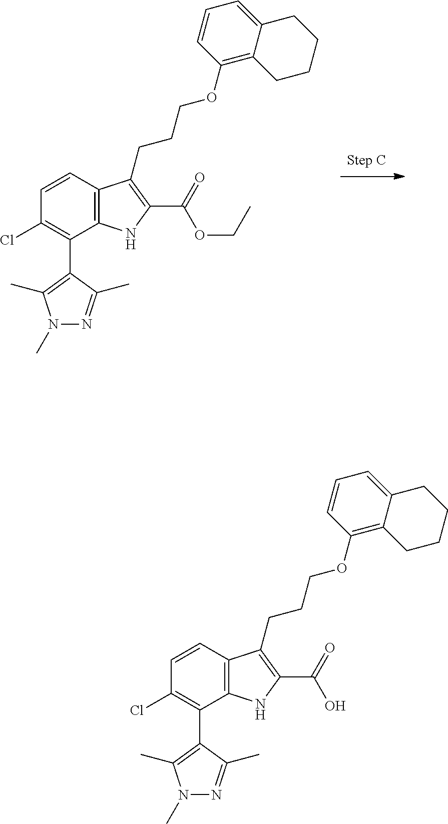 [SMILES: CCOC(=O)c1[nH]c2c(-c3c(C)nn(C)c3C)c(Cl)ccc2c1CCCOc1cccc2c1CCCC2 ; Cc1nn(C)c(C)c1-c1c(Cl)ccc2c(CCCOc3cccc4c3CCCC4)c(C(=O)O)[nH]c12]

Ethyl 6-chloro-3-(3-((5,6,7,8-tetrahydronaphthalen-1-yl)oxy)propyl)-7-(1,3,5-trimethyl-1H-pyrazol-4-yl)-1H-indole-2-carboxylate (4.0 g, 7.7 mmol) was dissolved in EtOH (50 mL) and a solution of NaOH (1.5 g, 38.5 mmol) in water (40 mL) was added to it. The mixture was heated under reflux for 16 h. After complete consumption of the starting material, the reaction mixture was cooled down to room temperature, solvents were evaporated under reduced pressure to get the crude reaction mixture. It was then diluted with water, washed with EtOAc. Aqueous layer was carefully acidified using 1M HCl to pH=3, extracted with EtOAc, the organic layer was dried over Na₂SO₄, filtered, and concentrated in vacuo to afford 3.3 g (6.7 mmol, 87%) of 6-chloro-3-(3-((5,6,7,8-tetrahydronaphthalen-1-yl)oxy)propyl)-7-(1,3,5-trimethyl-1H-pyrazol-4-yl)-1H-indole-2-carboxylic acid as off white solid.

LCMS (ESI+): m/z 492.1 [M+H]⁺

### Step D

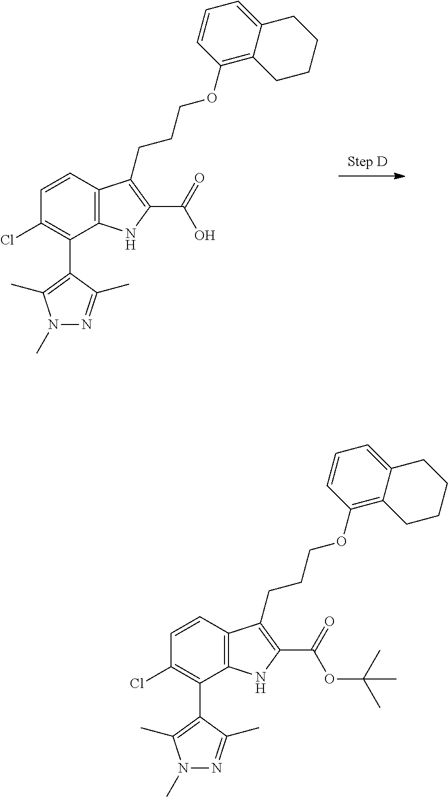 [SMILES: Cc1nn(C)c(C)c1-c1c(Cl)ccc2c(CCCOc3cccc4c3CCCC4)c(C(=O)O)[nH]c12 ; Cc1nn(C)c(C)c1-c1c(Cl)ccc2c(CCCOc3cccc4c3CCCC4)c(C(=O)OC(C)(C)C)[nH]c12]

6-chloro-7-(3,5-dimethyl-1H-pyrazol-4-yl)-3-(3-((5,6,7,8-tetrahydronaphthalen-1-yl)oxy)propyl)-1H-indole-2-carboxylic acid (3.3 g, 6.9 mmol) was suspended in toluene (50 ml) and the mixture was heated to reflux under nitrogen. N,N-dimethylformamide di-tert-butyl acetal (12 ml, 53.7 mmol) was added drop wise to the refluxing mixture. The mixture was heated under reflux for 16 h under nitrogen. After complete consumption of the starting material, the reaction mixture was then diluted with EtOAc, washed successively with aqueous NaHCO₃ (sat.), water and brine, organic layer was dried over Na₂SO₄ and evaporated under reduced pressure to get the crude compound, which was then purified by flash chromatography (SiO₂, 70% EtOAc in hexane) to get 3.3 g (6.0 mmol, 87%) of tert-butyl 6-chloro-3-(3-((5,6,7,8-tetrahydronaphthalen-1-yl)oxy)propyl)-7-(1,3,5-trimethyl-1H-pyrazol-4-yl)-1H-indole-2-carboxylate as brown solid.

LCMS (ESI+): m/z 548.4 [M+H]⁺

### Step E

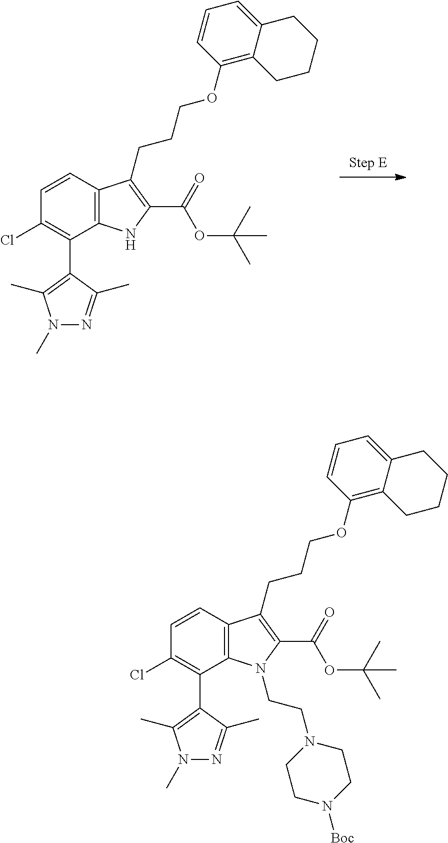 [SMILES: Cc1nn(C)c(C)c1-c1c(Cl)ccc2c(CCCOc3cccc4c3CCCC4)c(C(=O)OC(C)(C)C)[nH]c12 ; Cc1nn(C)c(C)c1-c1c(Cl)ccc2c(CCCOc3cccc4c3CCCC4)c(C(=O)OC(C)(C)C)n(CCN3CCN(C(=O)OC(C)(C)C)CC3)c12]

To a well stirred solution of tert-butyl 6-chloro-3-(3-((5,6,7,8-tetrahydronaphthalen-1-yl)oxy)propyl)-7-(1,3,5-trimethyl-1H-pyrazol-4-yl)-1H-indole-2-carboxylate (3.3 g, 6.0 mmol) in DMF (30 mL) was added tert-butyl 4-(2-chloroethyl)piperazine-1-carboxylate (3.0 g, 12.1 mmol) followed by Cs₂CO₃ (9.8 g, 30.2 mmol) in DMF (10 mL) and the mixture was allowed to stir at 90° C. for 16 h under nitrogen. After complete consumption of the starting material, the reaction mixture was diluted with EtOAc, washed successively with water and brine. The organic layer was dried over Na₂SO₄ and evaporated under reduced pressure to get the crude compound, which was then purified by flash chromatography (SiO₂, 30% EtOAc in hexane) to get 3.0 g (3.95 mmol, 66%) of tert-butyl 1-(2-(4-(tert-butoxycarbonyl)piperazin-1-yl)ethyl)-6-chloro-3-(3-((5,6,7,8-tetrahydronaphthalen-1-yl)oxy)propyl)-7-(1,3,5-trimethyl-1H-pyrazol-4-yl)-1H-indole-2-carboxylate as off white solid.

LCMS (ESI+): m/z 761.1 [M+H]⁺

### Step F

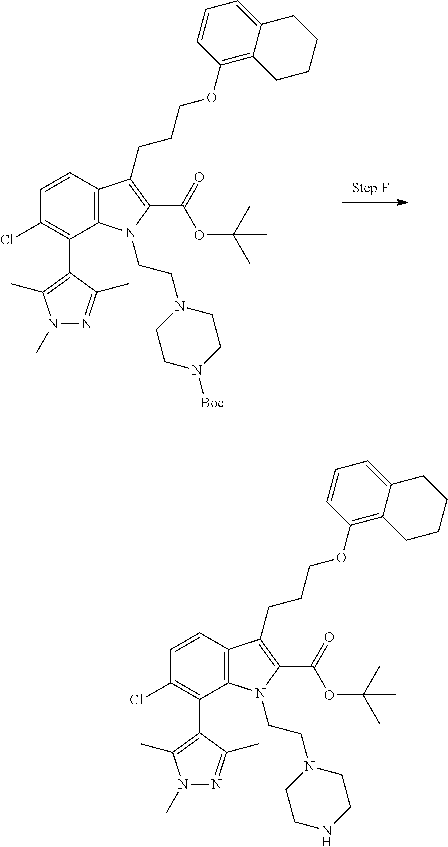 [SMILES: Cc1nn(C)c(C)c1-c1c(Cl)ccc2c(CCCOc3cccc4c3CCCC4)c(C(=O)OC(C)(C)C)n(CCN3CCN(C(=O)OC(C)(C)C)CC3)c12 ; Cc1nn(C)c(C)c1-c1c(Cl)ccc2c(CCCOc3cccc4c3CCCC4)c(C(=O)OC(C)(C)C)n(CCN3CCNCC3)c12]

Tert-butyl 1-(2-(4-(tert-butoxycarbonyl)piperazin-1-yl)ethyl)-6-chloro-3-(3-((5,6,7,8-tetrahydronaphthalen-1-yl)oxy)propyl)-7-(1,3,5-trimethyl-1H-pyrazol-4-yl)-1H-indole-2-carboxylate (3.0 g, 3.95 mmol) was dissolved in 50 mL of 4M HCl in dioxane at 0° C. and the mixture stirred for 2 h under nitrogen at same temperature. The reaction mixture was poured in to cold 1M NaOH solution and extracted several times with DCM. The combined organics were dried over Na₂SO₄ and concentrated in vacuo to get the crude compound which was then purified by flash chromatography (amine SiO₂, 10% MeOH in DCM) to get 1.54 g (2.33 mmol, 60%) of tert-butyl 6-chloro-1-(2-(piperazin-1-yl)ethyl)-3-(3-((5,6,7,8-tetrahydronaphthalen-1-yl)oxy)propyl)-7-(1,3,5-trimethyl-1H-pyrazol-4-yl)-1H-indole-2-carboxylate as white solid.

LCMS (ESI+): m/z 660.5 [M+H]⁺

### Step G

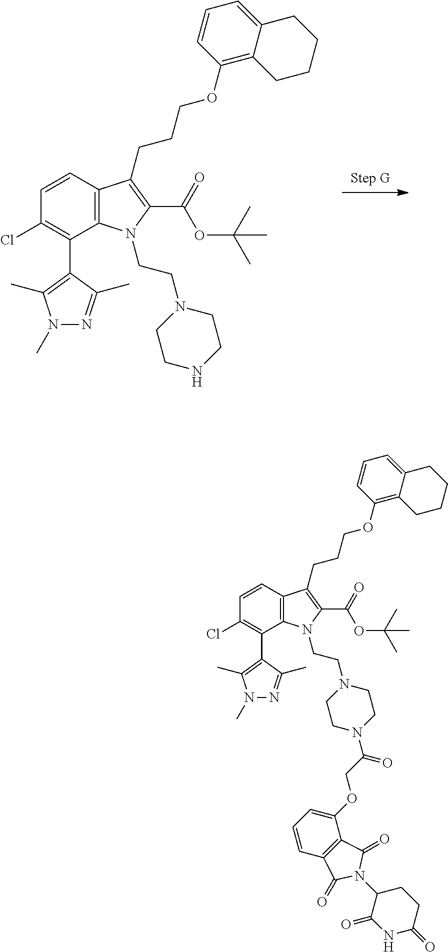 [SMILES: Cc1nn(C)c(C)c1-c1c(Cl)ccc2c(CCCOc3cccc4c3CCCC4)c(C(=O)OC(C)(C)C)n(CCN3CCNCC3)c12 ; Cc1nn(C)c(C)c1-c1c(Cl)ccc2c(CCCOc3cccc4c3CCCC4)c(C(=O)OC(C)(C)C)n(CCN3CCN(C(=O)COc4cccc5c4C(=O)N(C4CCC(=O)NC4=O)C5=O)CC3)c12]

2-{[2-(2,6-dioxopiperidin-3-yl)-1,3-dioxo-2,3-dihydro-1H-isoindol-4-yl]oxy}acetic acid (27.7 mg, 0.083 mmol) was dissolved in DMF (1.5 ml) under argon atmosphere. To it, CDI (18.4 mg, 0.114 mmol) was added and the reaction was stirred for 1 h in 60° C. Reaction mixture was then allowed to cool down to RT and a solution of tert-butyl 6-chloro-1-[2-(piperazin-1-yl)ethyl]-3-[3-(5,6,7,8-tetrahydronaphthalen-1-yloxy)propyl]-7-(1,3,5-trimethyl-1H-pyrazol-4-yl)-1H-indole-2-carboxylate (50.0 mg, 0.076 mmol) and DIPEA (0.040 mL, 0.227 mmol) was added in 1 ml of DMF. The reaction was stirred in RT for 72 h. After the reaction was completed (monitored with LCMS), DMF was evaporated, residue dissolved in DCM and washed with brine and water. Organic layer was dried over anhydrous Na₂SO₄, filtered, concentrated and dried under reduced pressure to give crude tert-butyl 6-chloro-1-{2-[4-(2-{[2-(2,6-dioxopiperidin-3-yl)-1,3-dioxo-2,3-dihydro-1H-isoindol-4-yl]oxy}acetyl)piperazin-1-yl]ethyl}-3-[3-(5,6,7,8-tetrahydronaphthalen-1-yloxy)propyl]-7-(1,3,5-trimethyl-1H-pyrazol-4-yl)-1H-indole-2-carboxylate (49.9 mg, crude) as yellow oil that was used in the next step without further purification.

LCMS (ESI+): m/z 973.9 [M+H]⁺

### Step H

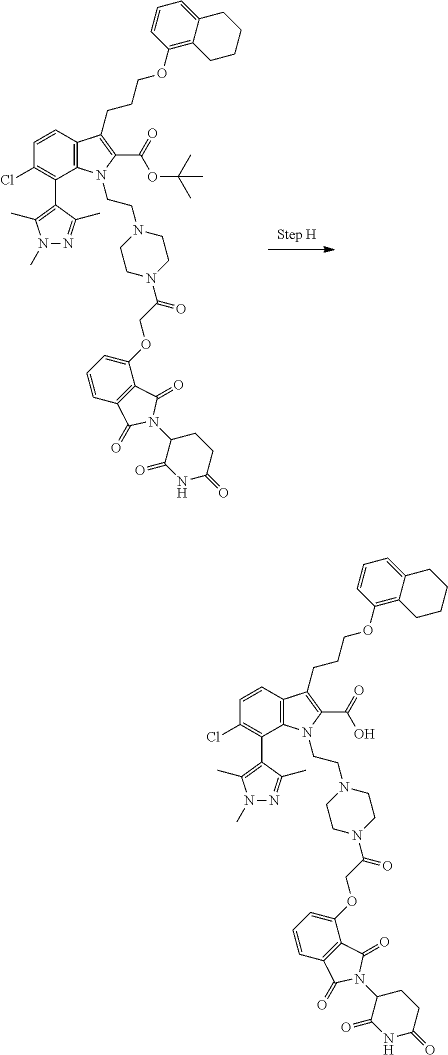 [SMILES: Cc1nn(C)c(C)c1-c1c(Cl)ccc2c(CCCOc3cccc4c3CCCC4)c(C(=O)OC(C)(C)C)n(CCN3CCN(C(=O)COc4cccc5c4C(=O)N(C4CCC(=O)NC4=O)C5=O)CC3)c12 ; Cc1nn(C)c(C)c1-c1c(Cl)ccc2c(CCCOc3cccc4c3CCCC4)c(C(=O)O)n(CCN3CCN(C(=O)COc4cccc5c4C(=O)N(C4CCC(=O)NC4=O)C5=O)CC3)c12]

Tert-butyl 6-chloro-1-{2-[4-(2-{[2-(2,6-dioxopiperidin-3-yl)-1,3-dioxo-2,3-dihydro-1H-isoindol-4-yl]oxy}acetyl)piperazin-1-yl]ethyl}-3-[3-(5,6,7,8-tetrahydronaphthalen-1-yloxy)propyl]-7-(1,3,5-trimethyl-1H-pyrazol-4-yl)-1H-indole-2-carboxylate (49.9 mg, crude) was dissolved in dry DCM (0.3 mL) under argon atmosphere and TFA (0.3 mL, 3.918 mmol) was added. Reaction was stirred for 16 h at room temperature under argon. After complete consumption of the starting material, solvent along with TFA were evaporated under reduced pressure and the resulting residue was dissolved in DMSO and purified by preparative HPLC (H₂O:MeCN+0.1% FA) to give the corresponding 6-chloro-1-{2-[4-(2-{[2-(2,6-dioxopiperidin-3-yl)-1,3-dioxo-2,3-dihydro-1H-isoindol-4-yl]oxy}acetyl)piperazin-1-yl]ethyl}-3-[3-(5,6,7,8-tetrahydronaphthalen-1-yloxy)propyl]-7-(1,3,5-trimethyl-1H-pyrazol-4-yl)-1H-indole-2-carboxylic acid (32.4 mg, 0.035 mmol, 46% over two steps) as white powder.

LCMS (ESI+): m/z 918.1 [M+H]⁺

¹H NMR (500 MHz, DMSO) δ 13.36 (s, 1H), 11.09 (s, 1H), 7.76 (dd, J=8.6, 7.3 Hz, 1H), 7.68 (d, J=8.5 Hz, 1H), 7.44 (d, J=7.2 Hz, 1H), 7.27 (dd, J=23.3, 8.6 Hz, 2H), 6.99 (t, J=7.8 Hz, 1H), 6.63 (t, J=7.2 Hz, 2H), 5.25-4.96 (m, 3H), 4.37-4.23 (m, 1H), 4.23-4.12 (m, 1H), 3.98 (t, J=6.2 Hz, 2H), 3.75 (s, 3H), 3.56-3.41 (m, 1H), 3.41-3.34 (m, 2H), 3.24-3.13 (m, 3H), 2.88 (ddd, J=16.9, 13.8, 5.5 Hz, 1H), 2.68 (t, J=6.1 Hz, 2H), 2.63-2.55 (m, 3H), 2.54-2.52 (m, 1H), 2.22-2.03 (m, 8H), 2.02 (s, 3H), 2.01-1.95 (m, 1H), 1.88 (s, 3H), 1.77-1.63 (m, 4H).

### Example 18: 6-chloro-3-(3-(4-chloro-3,5-dimethylphenoxy)propyl)-N-((2-(3-(2-(2-((2-(2,6-dioxopiperidin-3-yl)-1,3-dioxoisoindolin-4-yl)amino)ethoxy)ethoxy)propanamido)ethyl)sulfonyl)-1-methyl-7-(1,3,5-trimethyl-1H-pyrazol-4-yl)-1H-indole-2-carboxamide (252)

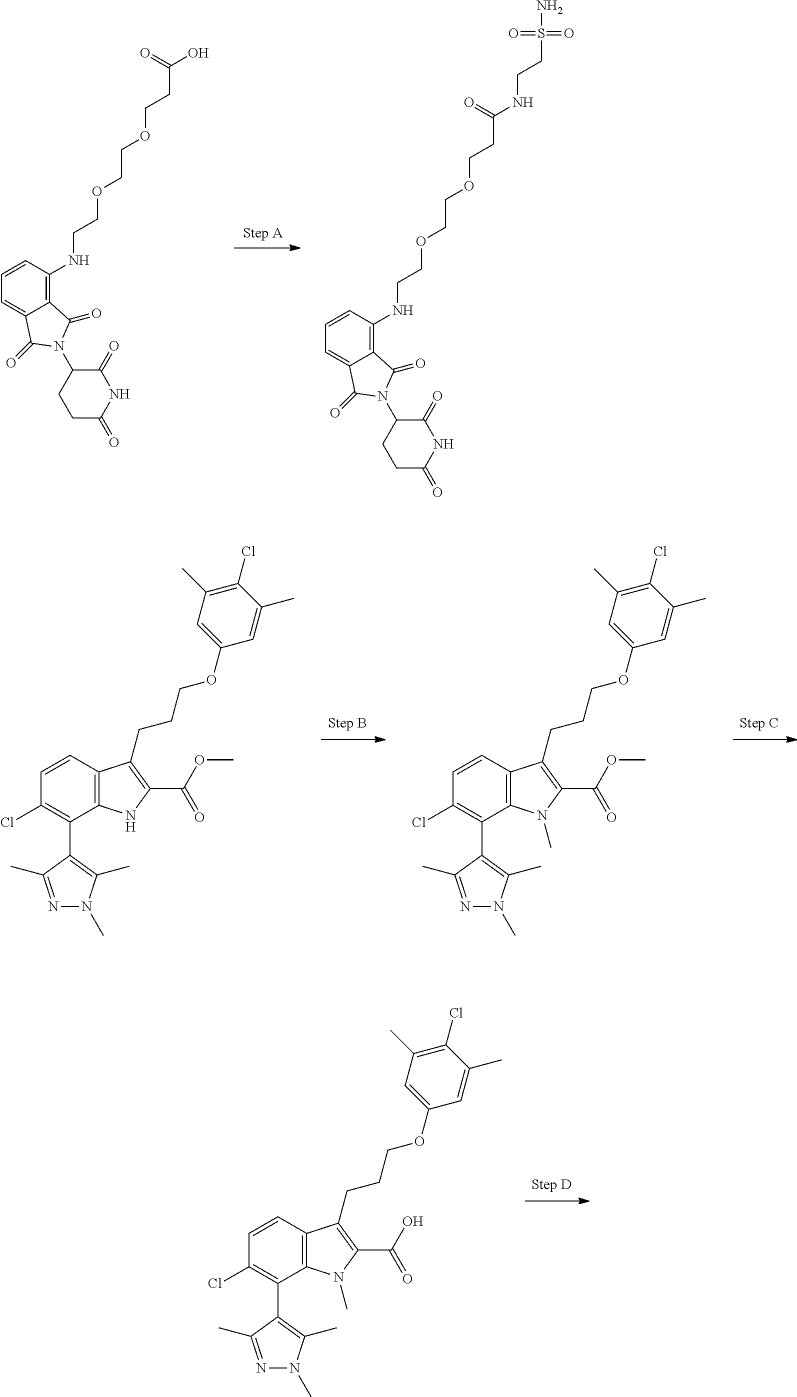

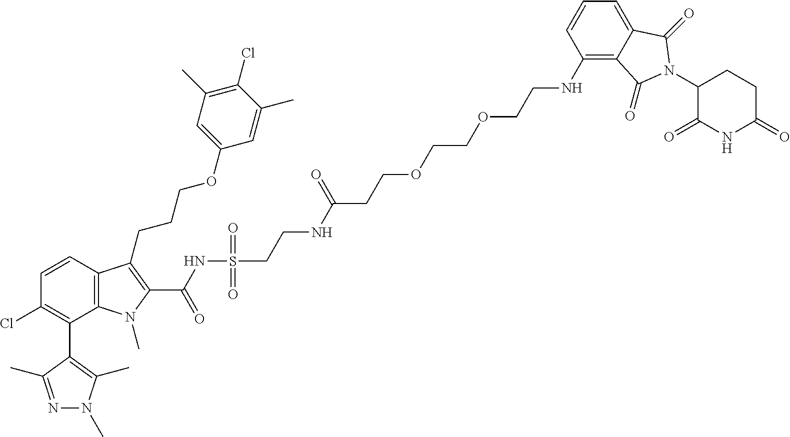 [SMILES: Cc1cc(OCCCc2c(C(=O)NS(=O)(=O)CCNC(=O)CCOCCOCCNc3cccc4c3C(=O)N(C3CCC(=O)NC3=O)C4=O)n(C)c3c(-c4c(C)nn(C)c4C)c(Cl)ccc23)cc(C)c1Cl]

### Step A

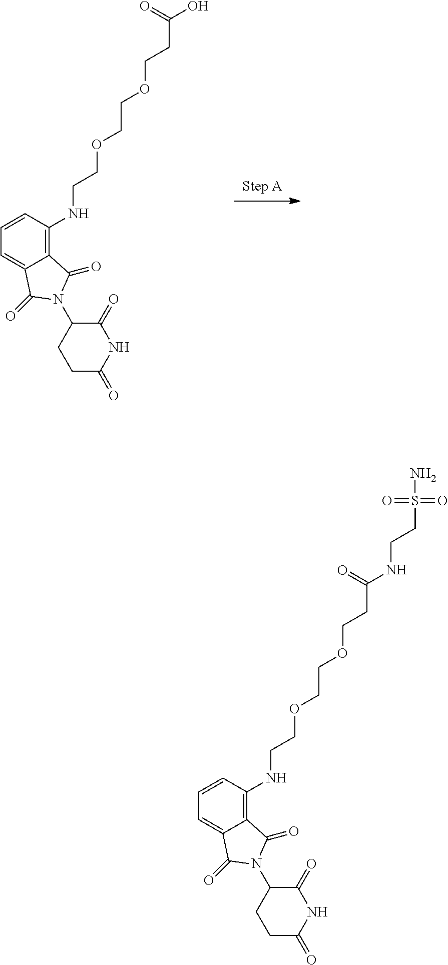 [SMILES: O=C(O)CCOCCOCCNc1cccc2c1C(=O)N(C1CCC(=O)NC1=O)C2=O ; NS(=O)(=O)CCNC(=O)CCOCCOCCNc1cccc2c1C(=O)N(C1CCC(=O)NC1=O)C2=O]

3-(2-(2-((2-(2,6-dioxopiperidin-3-yl)-1,3-dioxoisoindolin-4-yl)amino)ethoxy)ethoxy)propanoic acid (195.0 mg, 0.450 mmol) and HATU (205.3 mg, 0.540 mmol) were flashed with argon and dissolved in dry DMF (2.2 mL). DIPEA (0.235 mL, 1.350 mmol) was then added and the mixture stirred for 1 h in RT. After this time, 2-aminoethane-1-sulfonamide hydrochloride (72.3 mg, 0.450 mmol) was added and the reaction was stirred at RT for 1 hour. Solvents were removed under reduced pressure, aqueous NH₄Cl (saturated) and crude product was extracted with DCM. Desired product was purified using flash chromatography (SiO₂, DCM:MeOH, 0-10%) to get 3-(2-(2-((2-(2,6-dioxopiperidin-3-yl)-1,3-dioxoisoindolin-4-yl)amino)ethoxy)ethoxy)-N-(2-sulfamoylethyl)propanamide (135.0 mg, 0.250 mmol, 55.6%) as yellow oil.

LCMS (ESI+): m/z 540.4 [M+H]⁺

### Step B

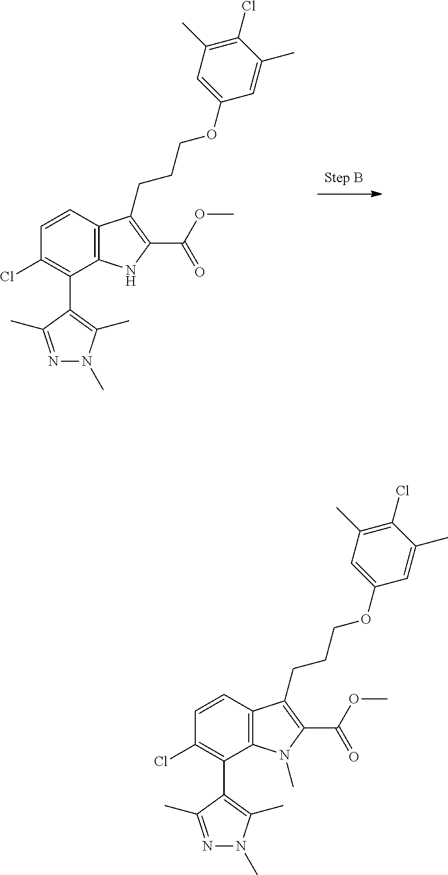

Iodomethane (0.133 mL, 2.138 mmol) was added to a stirred mixture of methyl 6-chloro-3-(3-(4-chloro-3,5-dimethylphenoxy)propyl)-7-(1,3,5-trimethyl-1H-pyrazol-4-yl)-1H-indole-2-carboxylate (1.000 g, 1.944 mmol) in DMF (6.0 mL), followed by K₂CO₃ (0.672 g, 4.860 mmol). The resulting mixture was stirred at room temperature for 24 hours. After the reaction was completed solvents were evaporated and the resulting residue was partitioned between EtOAc and H₂O. The organic layer was further washed with brine, dried over Na₂SO₄, filtered and evaporated. The methyl 6-chloro-3-(3-(4-chloro-3,5-dimethylphenoxy)propyl)-1-methyl-7-(1,3,5-trimethyl-1H-pyrazol-4-yl)-1H-indole-2-carboxylate (0.975 g, 1.845 mmol, 95%) was obtained as an orangish solid.

LCMS (ESI+): m/z 528.2 [M+H]⁺

### Step C

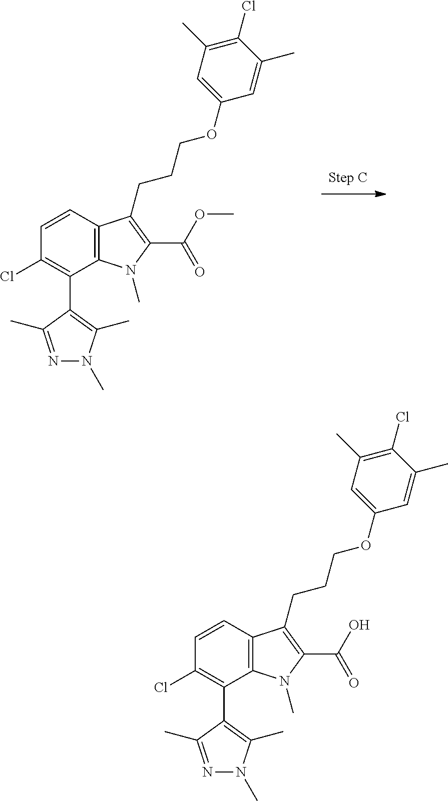

NaOH (5.4 mL, 5.450 mmol) was added to a stirred mixture of methyl 6-chloro-3-(3-(4-chloro-3,5-dimethylphenoxy)propyl)-1-methyl-7-(1,3,5-trimethyl-1H-pyrazol-4-yl)-1H-indole-2-carboxylate (0.720 g, 1.362 mmol) in MeOH (6.0 mL). The resulting mixture was stirred at room temperature for 20 hours and 4 h in 45° C. After the reaction was completed (monitored by LCMS), solvents were evaporated and the resulting residue was dissolved in DCM and H₂O. 1M HCl was added to achieve pH=˜3 of aqueous layer. The organic layer was further washed with brine, dried over Na₂SO₄, filtered and evaporated. The final product 6-chloro-3-(3-(4-chloro-3,5-dimethylphenoxy)propyl)-1-methyl-7-(1,3,5-trimethyl-1H-pyrazol-4-yl)-1H-indole-2-carboxylic acid (0.500 g, crude) was obtained as a white powder.

LCMS (ESI−): m/z 512.4 [M−H]⁻

### Step D

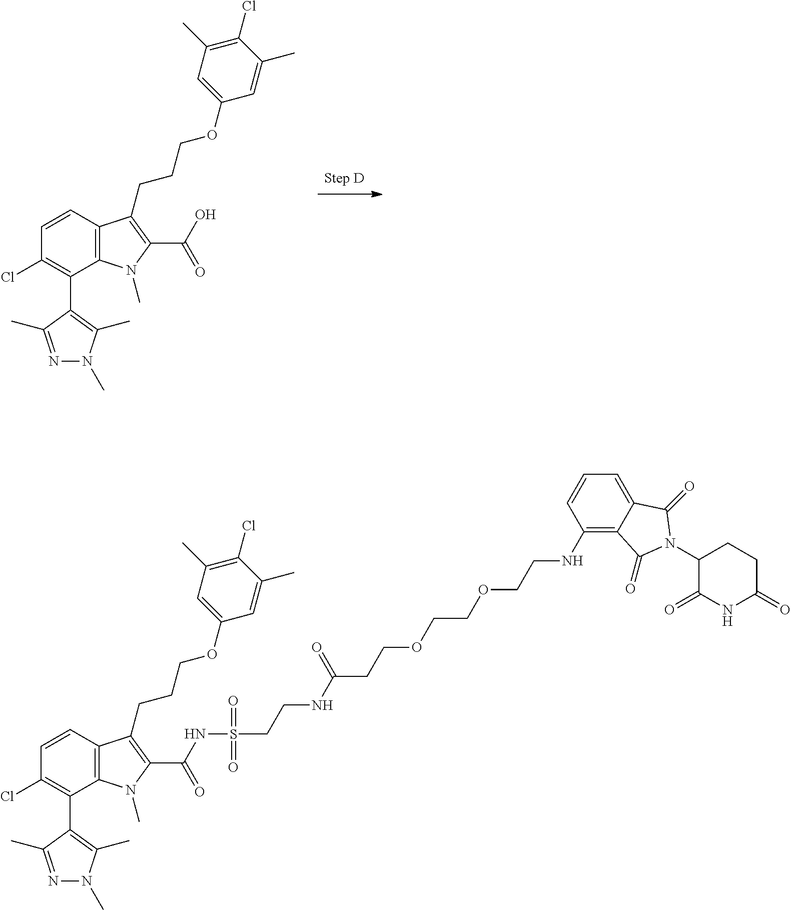

To a stirred solution of 3-(2-(2-((2-(2,6-dioxopiperidin-3-yl)-1,3-dioxoisoindolin-4-yl)amino)ethoxy)ethoxy)-N-(2-sulfamoylethyl)propanamide (20.0 mg, crude) in DCM (0.371 mL) were added EDC (7.1 mg, 0.037 mmol), DMAP (13.6 mg, 0.111 mmol), Et₃N (0.015 mL, 0.111 mmol) and 6-chloro-3-(3-(4-chloro-3,5-dimethylphenoxy)propyl)-1-methyl-7-(1,3,5-trimethyl-1H-pyrazol-4-yl)-1H-indole-2-carboxylic acid (18.1 mg, 0.035 mmol). The reaction mixture was stirred for 15 h at ambient temperature and then reaction was continued in 60° C. overnight. After this time solvents were removed under reduces pressure. The residue was dissolved in 1:1 mixture of DMSO:MeOH and filtered. The filtrate was purified by preparative HPLC (H₂O:MeCN+0.1% FA) to give a corresponding 6-chloro-3-(3-(4-chloro-3,5-dimethylphenoxy)propyl)-N-((2-(3-(2-(2-((2-(2,6-dioxopiperidin-3-yl)-1,3-dioxoisoindolin-4-yl)amino)ethoxy)ethoxy)propanamido)ethyl)sulfonyl)-1-methyl-7-(1,3,5-trimethyl-1H-pyrazol-4-yl)-1H-indole-2-carboxamide (1.0 mg, 0.001 mmol, 2.6%) as a yellow solid.

LCMS (ESI+): m/z 1034.9 [M+H]⁺

¹H NMR (500 MHz, DMSO) δ 12.28 (s, 1H), 11.07 (s, 1H), 8.09 (s, 1H), 7.67 (s, 1H), 7.56 (dd, J=8.6, 7.1 Hz, 1H), 7.24 (s, 1H), 7.12 (d, J=8.6 Hz, 1H), 7.02 (d, J=7.0 Hz, 1H), 6.75 (s, 2H), 6.58 (t, J=5.8 Hz, 1H), 5.04 (dd, J=12.7, 5.4 Hz, 1H), 3.94 (t, J=6.5 Hz, 2H), 3.75 (s, 3H), 3.72-3.54 (m, 6H), 3.54-3.48 (m, 3H), 3.48-3.42 (m, 6H), 3.03 (t, J=7.5 Hz, 2H), 2.87 (ddd, J=16.7, 13.7, 5.4 Hz, 1H), 2.60-2.54 (m, 4H), 2.30-2.24 (m, 8H), 2.05-1.96 (m, 6H), 1.88 (s, 3H).

### Example 19: 1-{2-[4-(2-{[2-(2,6-dioxopiperidin-3-yl)-1,3-dioxo-2,3-dihydro-1H-isoindol-4-yl]oxy}acetyl)piperazin-1-yl]ethyl}-6-fluoro-3-[3-(naphthalen-1-yloxy)propyl]-7-(1,3,5-trimethyl-1H-pyrazol-4-yl)-1H-indole-2-carboxylic acid (213)

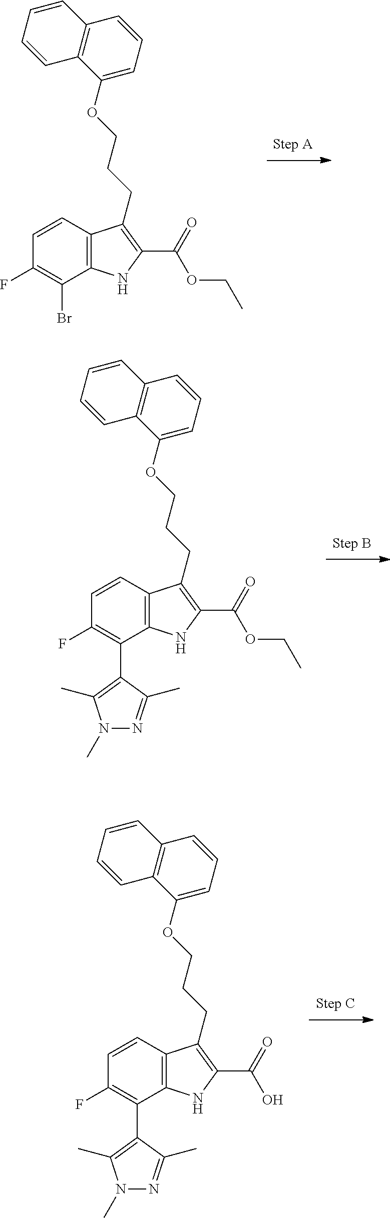

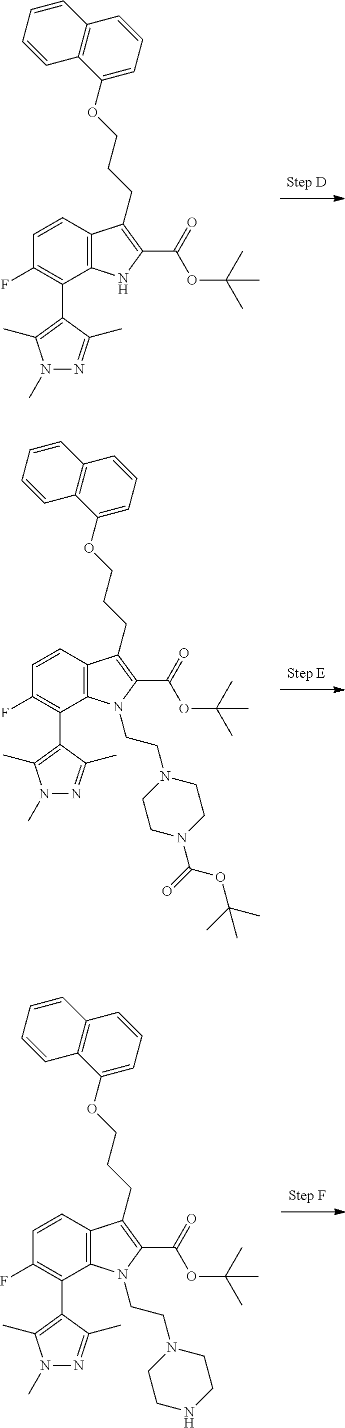

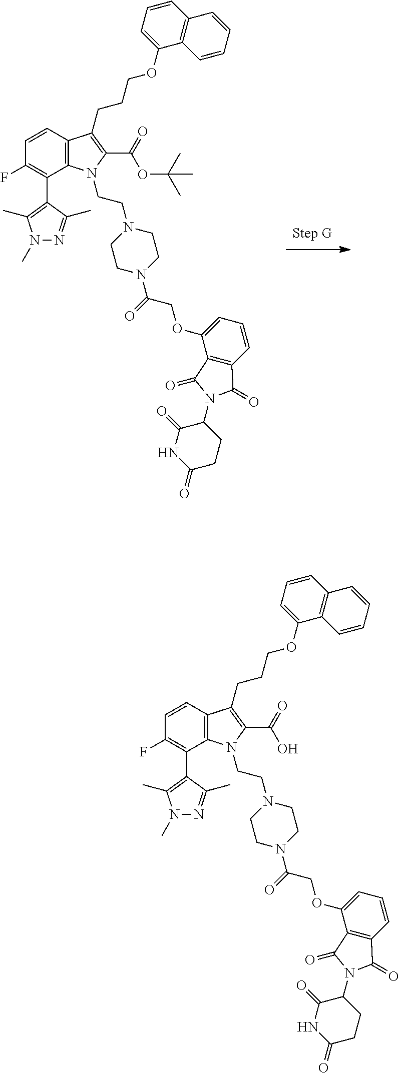 [SMILES: Cc1nn(C)c(C)c1-c1c(F)ccc2c(CCCOc3cccc4ccccc34)c(C(=O)OC(C)(C)C)n(CCN3CCN(C(=O)COc4cccc5c4C(=O)N(C4CCC(=O)NC4=O)C5=O)CC3)c12 ; Cc1nn(C)c(C)c1-c1c(F)ccc2c(CCCOc3cccc4ccccc34)c(C(=O)O)n(CCN3CCN(C(=O)COc4cccc5c4C(=O)N(C4CCC(=O)NC4=O)C5=O)CC3)c12]

### Step A

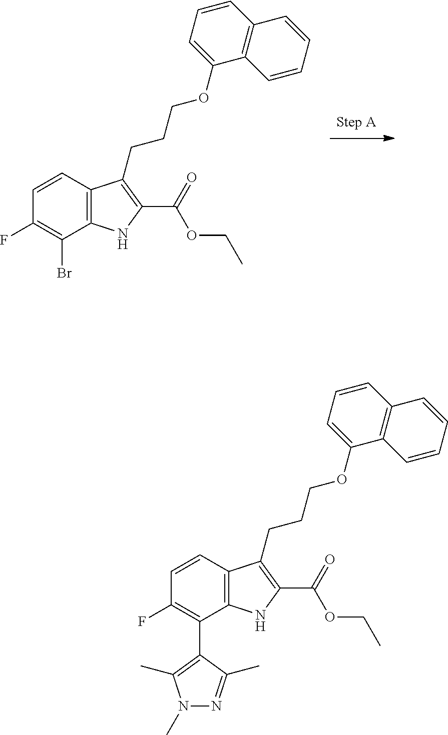 [SMILES: CCOC(=O)c1[nH]c2c(Br)c(F)ccc2c1CCCOc1cccc2ccccc12 ; CCOC(=O)c1[nH]c2c(-c3c(C)nn(C)c3C)c(F)ccc2c1CCCOc1cccc2ccccc12]

To a stirred solution of ethyl 7-bromo-6-fluoro-3-(3-(naphthalen-1-yloxy)propyl)-1H-indole-2-carboxylate (2.689 g, 5.717 mmol) in dioxane (25 mL) and water (5 mL) were added 1,3,5-trimethyl-4-(4,4,5,5-tetramethyl-1,3,2-dioxaborolan-2-yl)-1H-pyrazole (4.062 g, 17.2 mmol) and K₂CO₃ (3.165 g, 22.9 mmol) successively at RT. The mixture was deoxygenated with argon and to it was added Pd(dppf)Cl₂ (0.629 g, 0.86 mmol) under argon atmosphere. Then the reaction mixture was heated under reflux for 16 h. After complete consumption of the starting material the reaction mixture was filtered over celite and the filtrate was evaporated under reduced pressure to get the crude material. It was then diluted with EtOAc, washed successively with water and brine, the organic layer was dried over sodium sulphate and evaporated under reduced pressure to get the crude compound, which was purified by column chromatography (SiO₂, 50% EtOAc in hexane) to get ethyl 6-fluoro-3-(3-(naphthalen-1-yloxy)propyl)-7-(1,3,5-trimethyl-1H-pyrazol-4-yl)-1H-indole-2-carboxylate (2.125 g, 4.254 mmol, 74.4%) as brown solid.

LCMS (ESI+): m/z 500.0 [M+H]⁺

### Step B

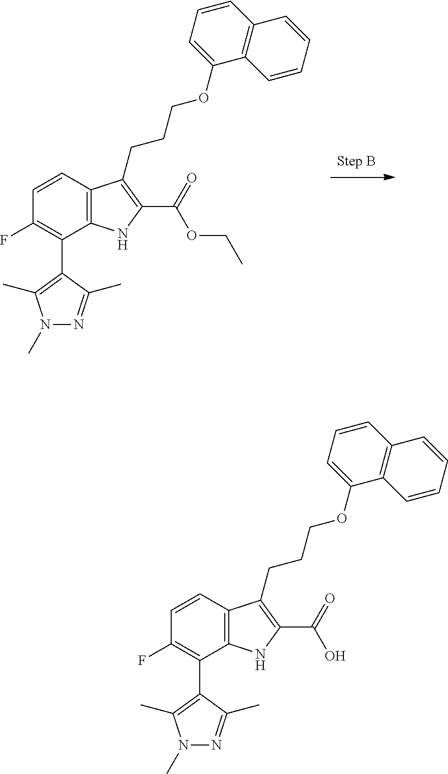 [SMILES: CCOC(=O)c1[nH]c2c(-c3c(C)nn(C)c3C)c(F)ccc2c1CCCOc1cccc2ccccc12 ; Cc1nn(C)c(C)c1-c1c(F)ccc2c(CCCOc3cccc4ccccc34)c(C(=O)O)[nH]c12]

Ethyl 6-fluoro-3-(3-(naphthalen-1-yloxy)propyl)-7-(1,3,5-trimethyl-1H-pyrazol-4-yl)-1H-indole-2-carboxylate (2.65 g, 5.30 mmol) was dissolved in EtOH (20 mL) and solution of NaOH (0.743 g, 18.58 mmol) in water (10 mL) was added to it. The mixture was heated under reflux for 16 h. After complete consumption of the starting material the reaction mixture was cooled down to room temperature, solvents were evaporated under reduced pressure to get the crude reaction mixture. It was then diluted with water, washed with EtOAc. Aqueous layer was carefully acidified using 1 (N) HCl to maintain pH=3, extracted with EtOAc, dried over Na₂SO₄, filtered, and concentrated under vacuum to afford 6-fluoro-3-(3-(naphthalen-1-yloxy)propyl)-7-(1,3,5-trimethyl-1H-pyrazol-4-yl)-1H-indole-2-carboxylic acid (2.3 g, crude) as dark brown gummy solid.

LCMS (ESI+): m/z 471.9 [M+H]⁺

### Step C

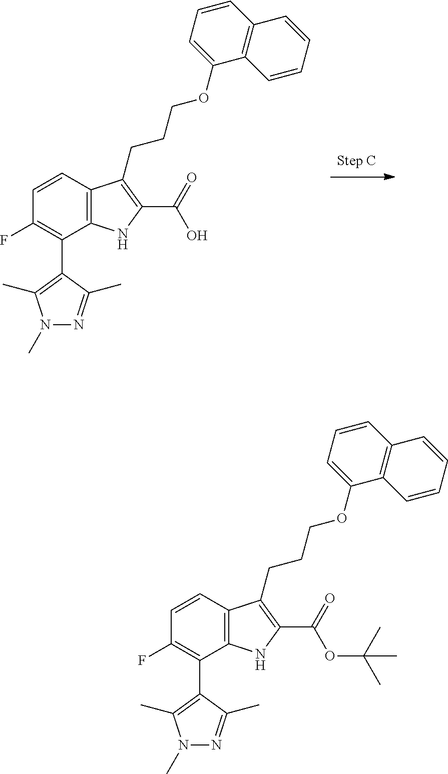 [SMILES: Cc1nn(C)c(C)c1-c1c(F)ccc2c(CCCOc3cccc4ccccc34)c(C(=O)O)[nH]c12 ; Cc1nn(C)c(C)c1-c1c(F)ccc2c(CCCOc3cccc4ccccc34)c(C(=O)OC(C)(C)C)[nH]c12]

6-fluoro-3-(3-(naphthalen-1-yloxy)propyl)-7-(1,3,5-trimethyl-1H-pyrazol-4-yl)-1H-indole-2-carboxylic acid (1.428 g, crude) was suspended in toluene (20 mL) and the mixture was heated to reflux under nitrogen. N,N-dimethylformamide di-tert-butyl acetal (5.8 ml, 24.25 mmol) was added drop wise to the refluxing mixture. The mixture was heated under reflux for 16 h under nitrogen. After complete consumption of the starting material reaction mixture was diluted with EtOAc, washed successively with NaHCO₃ (sat), water and brine, organic layer was dried over Na₂SO₄ and evaporated under reduced pressure to get the crude compound, which was then purified by column chromatography (SiO₂, 70% EtOAc in hexane) to get tert-butyl 6-fluoro-3-(3-(naphthalen-1-yloxy)propyl)-7-(1,3,5-trimethyl-1H-pyrazol-4-yl)-1H-indole-2-carboxylate (1.4 g, 2.65 mmol, 87.5%) as brown solid.

LCMS (ESI+): m/z 528.35 [M+H]⁺

### Step D

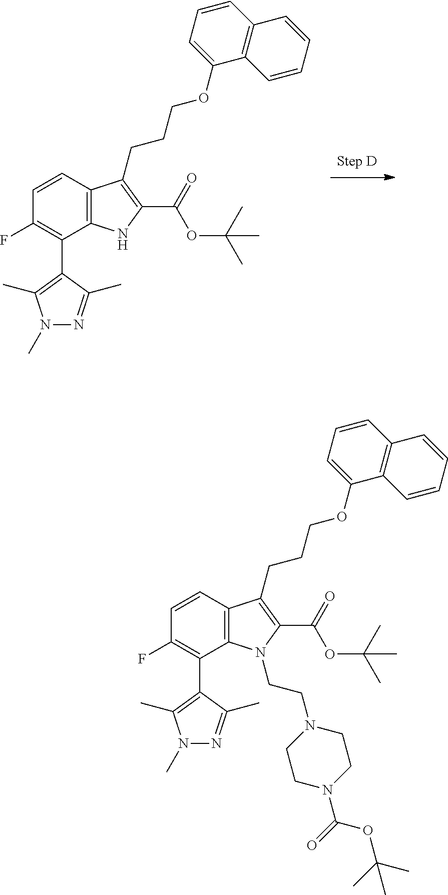 [SMILES: Cc1nn(C)c(C)c1-c1c(F)ccc2c(CCCOc3cccc4ccccc34)c(C(=O)OC(C)(C)C)[nH]c12 ; Cc1nn(C)c(C)c1-c1c(F)ccc2c(CCCOc3cccc4ccccc34)c(C(=O)OC(C)(C)C)n(CCN3CCN(C(=O)OC(C)(C)C)CC3)c12]

To a well stirred solution of tert-butyl 6-fluoro-3-(3-(naphthalen-1-yloxy)propyl)-7-(1,3,5-trimethyl-1H-pyrazol-4-yl)-1H-indole-2-carboxylate (1.86 g, 3.525 mmol) in DMF (20 mL) was added tert-butyl 4-(2-chloroethyl)piperazine-1-carboxylate (1.756 g, 7.059 mmol) followed by Cs₂CO₃ (5.735 g, 17.602 mmol) at RT under nitrogen. The reaction mixture was allowed to stir at 90° C. for 16 h under nitrogen. After complete consumption of the starting material the reaction mixture was diluted with EtOAc, washed successively with water and brine, the organic layer was dried over Na₂SO₄ and evaporated under reduced pressure to get the crude compound, which was then purified by column chromatography (SiO₂, 30% EtOAc in hexane) to get tert-butyl 1-(2-(4-(tert-butoxycarbonyl)piperazin-1-yl)ethyl)-6-fluoro-3-(3-(naphthalen-1-yloxy)propyl)-7-(1,3,5-trimethyl-1H-pyrazol-4-yl)-1H-indole-2-carboxylate (1.8 g, 2.434 mmol, 69.0%) as off white solid.

LCMS (ESI+): m/z 739.9 [M+H]⁺

### Step E

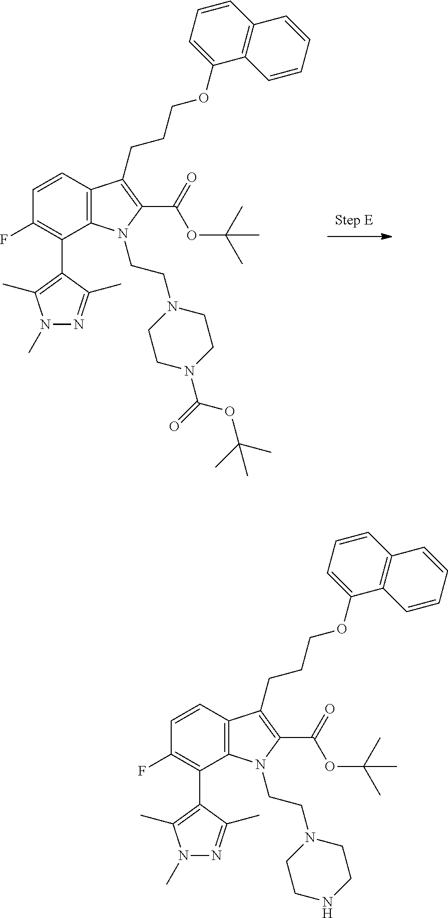 [SMILES: Cc1nn(C)c(C)c1-c1c(F)ccc2c(CCCOc3cccc4ccccc34)c(C(=O)OC(C)(C)C)n(CCN3CCN(C(=O)OC(C)(C)C)CC3)c12 ; Cc1nn(C)c(C)c1-c1c(F)ccc2c(CCCOc3cccc4ccccc34)c(C(=O)OC(C)(C)C)n(CCN3CCNCC3)c12]

Tert-butyl 1-(2-(4-(tert-butoxycarbonyl)piperazin-1-yl)ethyl)-6-fluoro-3-(3-(naphthalen-1-yloxy)propyl)-7-(1,3,5-trimethyl-1H-pyrazol-4-yl)-1H-indole-2-carboxylate (1.6 g, 2.164 mmol) was dissolved in 4 (M) HCl in dioxane (20 mL) at 0° C. and the mixture was stirred for 2 h under nitrogen at same temperature. Where upon LCMS indicated the reaction was completed, to the mixture was added drop wise cold 1M NaOH solution at 0° C. to maintain the pH˜5 to 6. The aqueous layer was extracted 2-3 times with DCM. The combined organics were dried over Na₂SO₄ and concentrated in vacuo to get the crude compound which was triturated with Et₂O to get tert-butyl 6-fluoro-3-(3-(naphthalen-1-yloxy)propyl)-1-(2-(piperazin-1-yl)ethyl)-7-(1,3,5-trimethyl-1H-pyrazol-4-yl)-1H-indole-2-carboxylate (1.03 g, 1.61 mmol, 74.4%) as white solid.

LCMS (ESI+): m/z 640.47 [M+H]⁺

### Step F

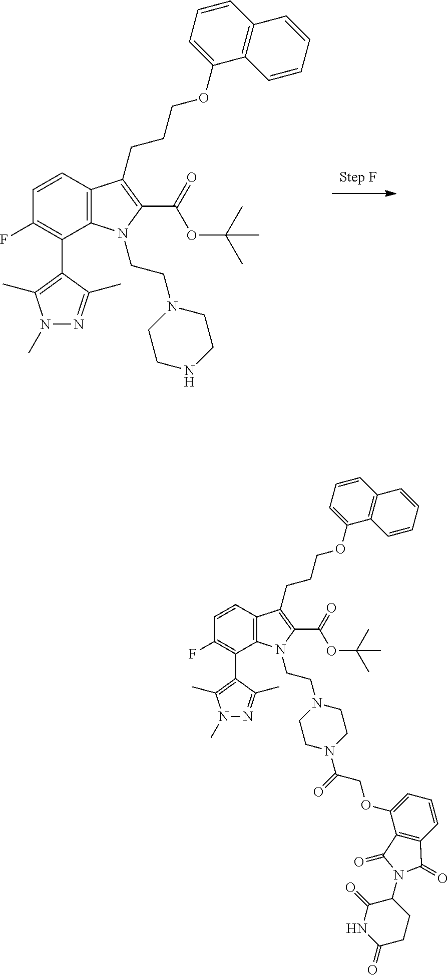 [SMILES: Cc1nn(C)c(C)c1-c1c(F)ccc2c(CCCOc3cccc4ccccc34)c(C(=O)OC(C)(C)C)n(CCN3CCNCC3)c12 ; Cc1nn(C)c(C)c1-c1c(F)ccc2c(CCCOc3cccc4ccccc34)c(C(=O)OC(C)(C)C)n(CCN3CCN(C(=O)COc4cccc5c4C(=O)N(C4CCC(=O)NC4=O)C5=O)CC3)c12]

To a solution of 2-{[2-(2,6-dioxopiperidin-3-yl)-1,3-dioxo-2,3-dihydro-1H-isoindol-4-yl]oxy}acetic acid (51.9 mg, 0.156 mmol) and tert-butyl 6-fluoro-3-[3-(naphthalen-1-yloxy)propyl]-1-[2-(piperazin-1-yl)ethyl]-7-(1,3,5-trimethyl-1H-pyrazol-4-yl)-1H-indole-2-carboxylate (50.0 mg, 0.078 mmol) in dry DMF (1.0 mL) was added DIPEA (0.041 mL, 0.234 mmol) and HATU (35.7 mg, 0.094 mmol). The mixture was stirred for 36 h at RT. After complete consumption of the starting material the solvent was evaporated and residues were dissolved in EtOAc. Organic phase was extracted with NaHCO₃ sat. and brine. Then, organic layer was dried over Na₂SO₄, filtered and concentrated under vacuum to get 30.0 mg crude of tert-butyl 1-{2-[4-(2-{[2-(2,6-dioxopiperidin-3-yl)-1,3-dioxo-2,3-dihydro-1H-isoindol-4-yl]oxy}acetyl)piperazin-1-yl]ethyl}-6-fluoro-3-[3-(naphthalen-1-yloxy)propyl]-7-(1,3,5-trimethyl-1H-pyrazol-4-yl)-1H-indole-2-carboxylate was used to next step without further purification.

LCMS (ESI+): m/z 954.23 [M+H]⁺

### Step G

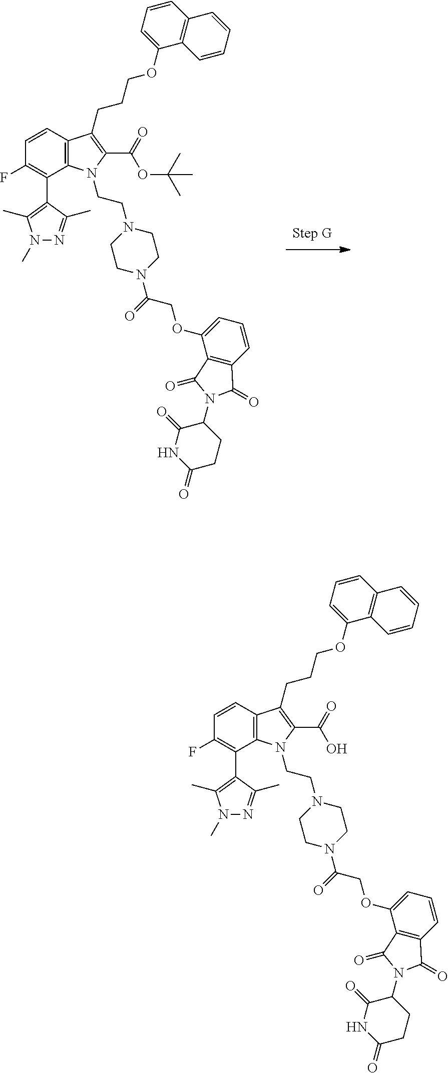 [SMILES: Cc1nn(C)c(C)c1-c1c(F)ccc2c(CCCOc3cccc4ccccc34)c(C(=O)OC(C)(C)C)n(CCN3CCN(C(=O)COc4cccc5c4C(=O)N(C4CCC(=O)NC4=O)C5=O)CC3)c12 ; Cc1nn(C)c(C)c1-c1c(F)ccc2c(CCCOc3cccc4ccccc34)c(C(=O)O)n(CCN3CCN(C(=O)COc4cccc5c4C(=O)N(C4CCC(=O)NC4=O)C5=O)CC3)c12]

To a solution of tert-butyl 1-{2-[4-(2-{[2-(2,6-dioxopiperidin-3-yl)-1,3-dioxo-2,3-dihydro-1H-isoindol-4-yl]oxy}acetyl)piperazin-1-yl]ethyl}-6-fluoro-3-[3-(naphthalen-1-yloxy)propyl]-7-(1,3,5-trimethyl-1H-pyrazol-4-yl)-1H-indole-2-carboxylate (30.0 mg, crude) in DCM (1.0 mL) was added TFA (1.0 mL, 13.059 mmol). The reaction was stirred for 18 h at RT. The crude was concentrated in vacuo, dissolved in DMSO and purified by preparative HPLC (H₂O:MeCN+0.1% FA) to give a final product 1-{2-[4-(2-{[2-(2,6-dioxopiperidin-3-yl)-1,3-dioxo-2,3-dihydro-1H-isoindol-4-yl]oxy}acetyl)piperazin-1-yl]ethyl}-6-fluoro-3-[3-(naphthalen-1-yloxy)propyl]-7-(1,3,5-trimethyl-1H-pyrazol-4-yl)-1H-indole-2-carboxylic acid (3.2 mg, 0.004 mmol, 2.6% over two steps) as a white solid.

LCMS (ESI+): m/z 898.29 [M+H]⁺

¹H NMR (500 MHz, DMSO) δ 13.32 (s, 1H), 11.09 (s, 1H), 8.21 (dd, J=7.7, 1.9 Hz, 1H), 7.89-7.84 (m, 1H), 7.82-7.73 (m, 2H), 7.55-7.48 (m, 2H), 7.45 (dd, J=7.7, 4.7 Hz, 2H), 7.43-7.37 (m, 1H), 7.31 (d, J=8.5 Hz, 1H), 7.08-6.99 (m, 1H), 6.94-6.88 (m, 1H), 5.17-5.05 (m, 3H), 4.40 (s, 1H), 4.33 (s, 1H), 4.22 (t, J=6.2 Hz, 2H), 3.76 (s, 3H), 3.30 (t, J=7.6 Hz, 4H), 2.88 (ddd, J=16.9, 14.5, 5.3 Hz, 1H), 2.62-2.52 (m, 2H), 2.28-2.16 (m, 4H), 2.08-2.00 (m, 6H), 1.93 (s, 3H).

4 protons in aliphatic area overlaps with water.

### Example 20: 1-{2-[4-(2-{[2-(2,6-dioxopiperidin-3-yl)-1,3-dioxo-2,3-dihydro-1H-isoindol-4-yl]oxy}acetyl)piperazin-1-yl]ethyl}-6-fluoro-3-{3-[(6-fluoronaphthalen-1-yl)oxy]propyl}-7-(1,3,5-trimethyl-1H-pyrazol-4-yl)-1H-indole-2-carboxylic acid (215)

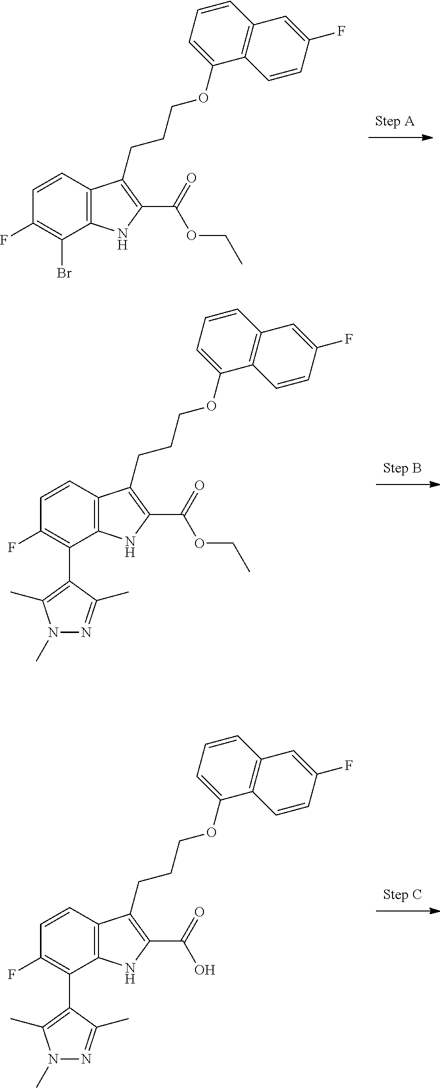

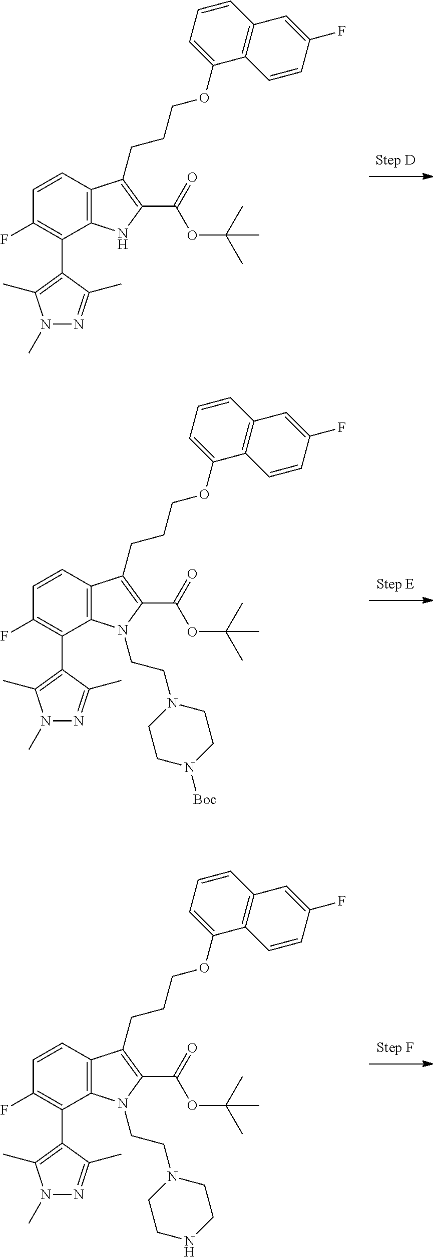 [SMILES: Cc1nn(C)c(C)c1-c1c(F)ccc2c(CCCOc3cccc4cc(F)ccc34)c(C(=O)OC(C)(C)C)[nH]c12 ; Cc1nn(C)c(C)c1-c1c(F)ccc2c(CCCOc3cccc4cc(F)ccc34)c(C(=O)OC(C)(C)C)n(CCN3CCN(C(=O)OC(C)(C)C)CC3)c12 ; Cc1nn(C)c(C)c1-c1c(F)ccc2c(CCCOc3cccc4cc(F)ccc34)c(C(=O)OC(C)(C)C)n(CCN3CCNCC3)c12]

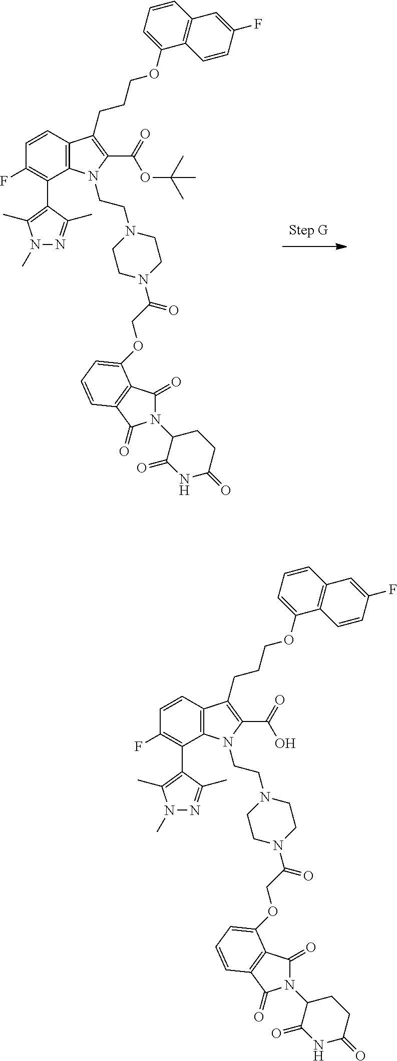 [SMILES: Cc1nn(C)c(C)c1-c1c(F)ccc2c(CCCOc3cccc4cc(F)ccc34)c(C(=O)OC(C)(C)C)n(CCN3CCN(C(=O)COc4cccc5c4C(=O)N(C4CCC(=O)NC4=O)C5=O)CC3)c12 ; Cc1nn(C)c(C)c1-c1c(F)ccc2c(CCCOc3cccc4cc(F)ccc34)c(C(=O)O)n(CCN3CCN(C(=O)COc4cccc5c4C(=O)N(C4CCC(=O)NC4=O)C5=O)CC3)c12]

### Step A

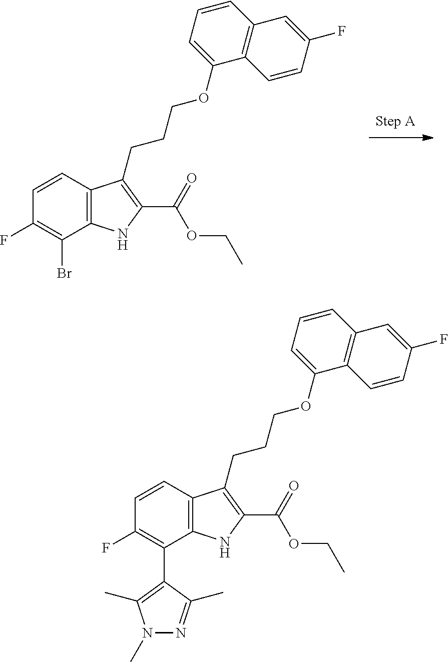 [SMILES: CCOC(=O)c1[nH]c2c(Br)c(F)ccc2c1CCCOc1cccc2cc(F)ccc12 ; CCOC(=O)c1[nH]c2c(-c3c(C)nn(C)c3C)c(F)ccc2c1CCCOc1cccc2cc(F)ccc12]

To a stirred solution of ethyl 7-bromo-6-fluoro-3-(3-((6-fluoronaphthalen-1-yl)oxy)propyl)-1H-indole-2-carboxylate (2.38 g, 4.87 mmol) in dioxane (25 mL) and water (5 mL), were added 1,3,5-trimethyl-4-(4,4,5,5-tetramethyl-1,3,2-dioxaborolan-2-yl)-1H-pyrazole (3.46 g, 14.6 mmol) and K₂CO₃ (2.7 g, 19.5 mmol) at room temperature in an inert atmosphere. To the mixture was added Pd(dppf)Cl₂ (0.54 g, 0.73 mmol). Then, the reaction mixture was heated under reflux for 16 h. After complete consumption of the starting material, the reaction mixture was filtered through celite pad and the filtrate was evaporated under reduced pressure to get the residue. It was then diluted with EtOAc and washed successively with water and brine solution. The organic layer was dried over Na₂SO₄ and evaporated under reduced pressure to get the crude compound, which was then triturated with Et₂O to get 2.2 g (crude) of ethyl 6-fluoro-3-(3-((6-fluoronaphthalen-1-yl)oxy)propyl)-7-(1,3,5-trimethyl-1H-pyrazol-4-yl)-1H-indole-2-carboxylate as a brown solid which was directly used in the next step without further purification.

LCMS (ESI+): m/z 518.3 [M+H]⁺

### Step B

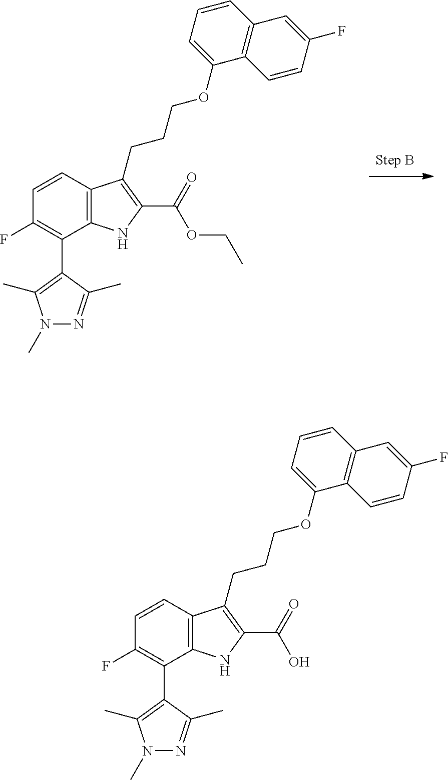 [SMILES: CCOC(=O)c1[nH]c2c(-c3c(C)nn(C)c3C)c(F)ccc2c1CCCOc1cccc2cc(F)ccc12 ; Cc1nn(C)c(C)c1-c1c(F)ccc2c(CCCOc3cccc4cc(F)ccc34)c(C(=O)O)[nH]c12]

The crude (3.8 g) of ethyl 6-fluoro-3-(3-((6-fluoronaphthalen-1-yl)oxy)propyl)-7-(1,3,5-trimethyl-1H-pyrazol-4-yl)-1H-indole-2-carboxylate was dissolved in EtOH (40 mL) and solution of NaOH (1.03 g, 25.7 mmol) in water (20 mL) was added to it. The mixture was heated under reflux for 16 h. After complete consumption of the starting material, the reaction mixture was cooled down to room temperature and solvents were evaporated under reduced pressure. It was then diluted with water and washed with EtOAc. Aqueous layer was carefully acidified using 1M HCl to pH=3, extracted with EtOAc, dried over Na₂SO₄, filtered, and concentrated in vacuo to afford 3.2 g crude of 6-fluoro-3-(3-((6-fluoronaphthalen-1-yl)oxy)propyl)-7-(1,3,5-trimethyl-1H-pyrazol-4-yl)-1H-indole-2-carboxylic acid as dark brown gummy solid which was directly used for next step.

LCMS (ESI+): m/z 490.0 [M+H]⁺

### Step C

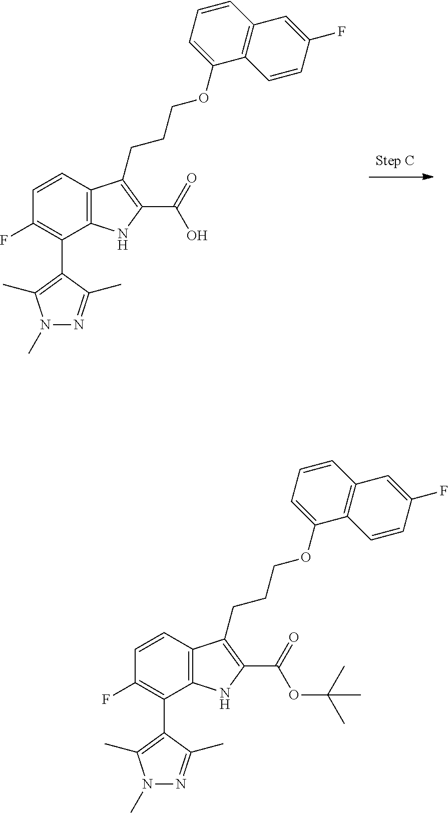 [SMILES: Cc1nn(C)c(C)c1-c1c(F)ccc2c(CCCOc3cccc4cc(F)ccc34)c(C(=O)O)[nH]c12 ; Cc1nn(C)c(C)c1-c1c(F)ccc2c(CCCOc3cccc4cc(F)ccc34)c(C(=O)OC(C)(C)C)[nH]c12]

6-fluoro-3-(3-((6-fluoronaphthalen-1-yl)oxy)propyl)-7-(1,3,5-trimethyl-1H-pyrazol-4-yl)-1H-indole-2-carboxylic acid (1.48 g of crude) was suspended in toluene (20 mL) and the mixture was heated to reflux under nitrogen. N,N-dimethylformamide di-tert-butyl acetal (5.82 ml, 24.3 mmol) was added drop wise to the refluxing mixture. The mixture was heated under reflux for 16 h under nitrogen. After complete consumption of the starting material, reaction mixture was then diluted with EtOAc, washed successively with NaHCO₃ (sat.), water and brine. Organic layer was dried over Na₂SO₄ and evaporated under reduced pressure to get the crude compound, which was then purified by flash chromatography (SiO₂, 70% EtOAc in hexane) to get tert-butyl 6-fluoro-3-(3-((6-fluoronaphthalen-1-yl)oxy)propyl)-7-(1,3,5-trimethyl-1H-pyrazol-4-yl)-1H-indole-2-carboxylate (1.42 g, 2.60 mmol, 53% over three steps) as brown solid.

LCMS (ESI+): m/z 546.5 [M+H]⁺

### Step D

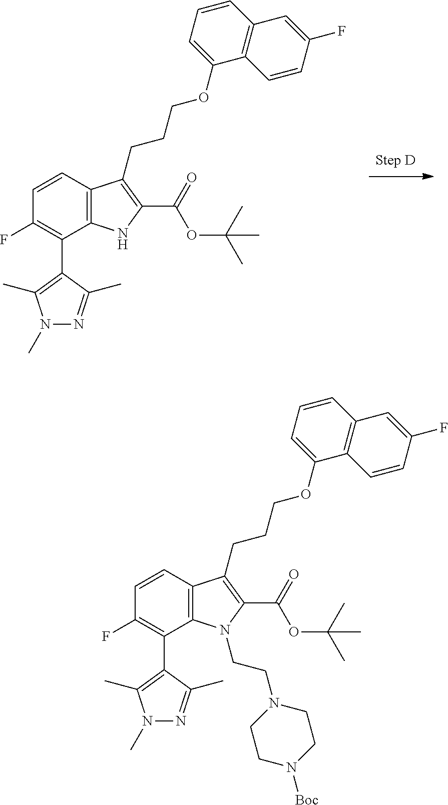 [SMILES: Cc1nn(C)c(C)c1-c1c(F)ccc2c(CCCOc3cccc4cc(F)ccc34)c(C(=O)OC(C)(C)C)[nH]c12 ; Cc1nn(C)c(C)c1-c1c(F)ccc2c(CCCOc3cccc4cc(F)ccc34)c(C(=O)OC(C)(C)C)n(CCN3CCN(C(=O)OC(C)(C)C)CC3)c12]

To a well stirred solution of tert-butyl 6-fluoro-3-(3-((6-fluoronaphthalen-1-yl)oxy)propyl)-7-(1,3,5-trimethyl-1H-pyrazol-4-yl)-1H-indole-2-carboxylate (1.0 g, 1.8 mmol) in DMF (10 mL) was added tert-butyl 4-(2-chloroethyl)piperazine-1-carboxylate (0.91 g, 3.7 mmol) followed by Cs₂CO₃ (2.98 g, 9.2 mmol) at room temperature under nitrogen. The resulting mixture was allowed to stir at 90° C. for 16 h under nitrogen. After complete consumption of the starting material, the reaction mixture was diluted with EtOAc and washed successively with water and brine. The organic layer was dried over Na₂SO₄ and evaporated under reduced pressure to get the crude compound, which was then purified by flash chromatography (SiO₂, 30% EtOAc in hexane) to get tert-butyl 1-(2-(4-(tert-butoxycarbonyl)piperazin-1-yl)ethyl)-6-fluoro-3-(3-((6-fluoronaphthalen-1-yl)oxy)propyl)-7-(1,3,5-trimethyl-1H-pyrazol-4-yl)-1H-indole-2-carboxylate (900 mg, 1.18 mmol, 65%) as off white solid.

LCMS (ESI+): m/z 757.8 [M+H]⁺

### Step E

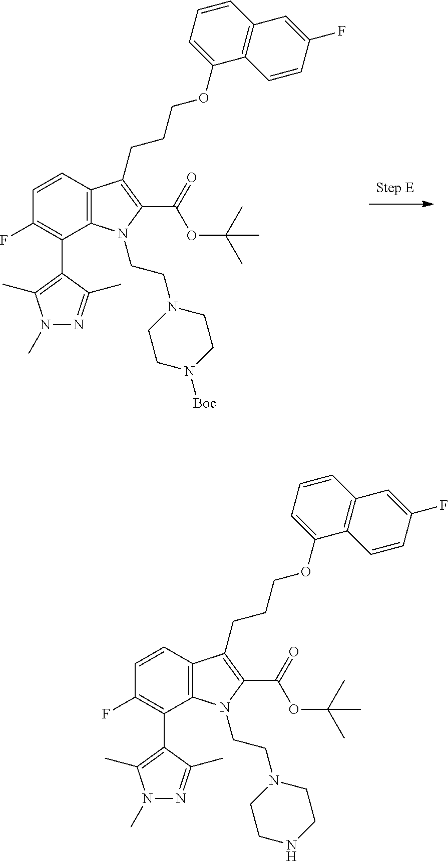 [SMILES: Cc1nn(C)c(C)c1-c1c(F)ccc2c(CCCOc3cccc4cc(F)ccc34)c(C(=O)OC(C)(C)C)n(CCN3CCN(C(=O)OC(C)(C)C)CC3)c12 ; Cc1nn(C)c(C)c1-c1c(F)ccc2c(CCCOc3cccc4cc(F)ccc34)c(C(=O)OC(C)(C)C)n(CCN3CCNCC3)c12]

Tert-butyl 1-(2-(4-(tert-butoxycarbonyl)piperazin-1-yl)ethyl)-6-fluoro-3-(3-((6-fluoronaphthalen-1-yl)oxy)propyl)-7-(1,3,5-trimethyl-1H-pyrazol-4-yl)-1H-indole-2-carboxylate (0.9 g, 1.2 mmol) dissolved in 4M HCl in dioxane (20 mL) at 0° C. and the mixture stirred for 2 h under nitrogen at the same temperature. Cold 1M NaOH was added drop-wise to the reaction mixture at 0° C. until pH=5-6 was reached. The aqueous layer was extracted 2-3 times with DCM, the combined organics were dried over Na₂SO₄ and concentrated in vacuum to get the crude compound. Reaction product was purified by preparative HPLC (10 mM ammonium acetate in H₂O:MeCN) to get of tert-butyl 6-fluoro-3-(3-((6-fluoronaphthalen-1-yl)oxy)propyl)-1-(2-(piperazin-1-yl)ethyl)-7-(1,3,5-trimethyl-1H-pyrazol-4-yl)-1H-indole-2-carboxylate (225 mg, 0.34 mmol, 28%) as off white solid.

LCMS (ESI+): m/z 658.5 [M+H]⁺

### Step F

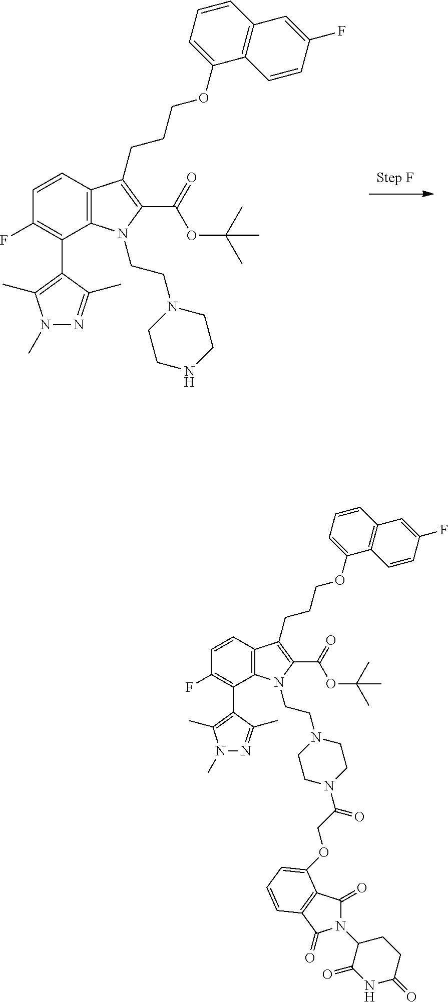 [SMILES: Cc1nn(C)c(C)c1-c1c(F)ccc2c(CCCOc3cccc4cc(F)ccc34)c(C(=O)OC(C)(C)C)n(CCN3CCNCC3)c12 ; Cc1nn(C)c(C)c1-c1c(F)ccc2c(CCCOc3cccc4cc(F)ccc34)c(C(=O)OC(C)(C)C)n(CCN3CCN(C(=O)COc4cccc5c4C(=O)N(C4CCC(=O)NC4=O)C5=O)CC3)c12]

2-{[2-(2,6-dioxopiperidin-3-yl)-1,3-dioxo-2,3-dihydro-1H-isoindol-4-yl]oxy}acetic acid (16.7 mg, 0.050 mmol) was dissolved in dry DMF (0.91 ml) under argon atmosphere and DIPEA (0.024 mL, 0.137 mmol,), followed by HATU (26.0 mg, 0.068 mmol) were added. After 15 min of mixing at room temperature tert-butyl 6-fluoro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (30.0 mg, 0.046 mmol) was added. Reaction was continued for 16 h at room temperature. Solvents were evaporated, resulting material dissolved in DCM (30 ml) and successfully washed with brine and water. Organic layer was dried over anhydrous Na₂SO₄, filtered, concentrated and dried under reduced pressure. Resulting product, tert-butyl 1-(2-(4-(2-((2-(2,6-dioxopiperidin-3-yl)-1,3-dioxoisoindolin-4-yl)oxy)acetyl)piperazin-1-yl)ethyl)-6-fluoro-3-(3-((6-fluoronaphthalen-1-yl)oxy)propyl)-7-(1,3,5-trimethyl-1H-pyrazol-4-yl)-1H-indole-2-carboxylate (44 mg, crude) was used in the next step without further purification.

LCMS (ESI+): m/z 972.2 [M+H]⁺

### Step G

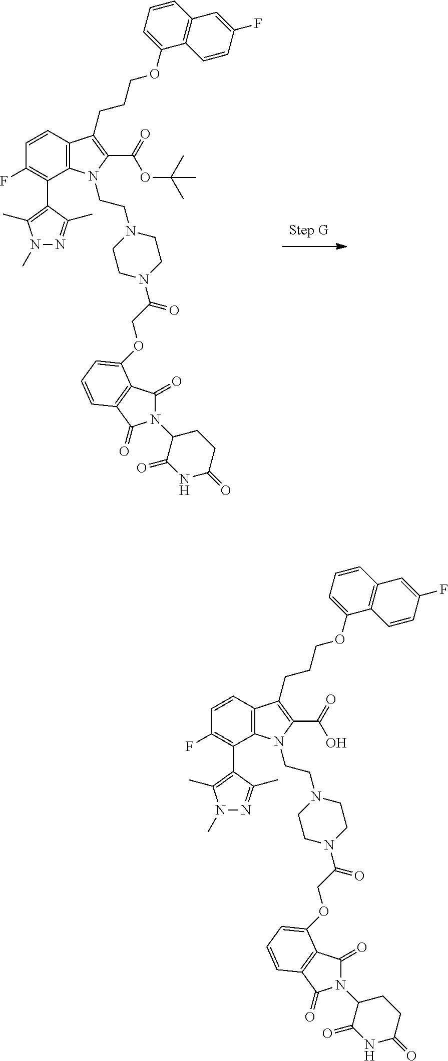 [SMILES: Cc1nn(C)c(C)c1-c1c(F)ccc2c(CCCOc3cccc4cc(F)ccc34)c(C(=O)OC(C)(C)C)n(CCN3CCN(C(=O)COc4cccc5c4C(=O)N(C4CCC(=O)NC4=O)C5=O)CC3)c12 ; Cc1nn(C)c(C)c1-c1c(F)ccc2c(CCCOc3cccc4cc(F)ccc34)c(C(=O)O)n(CCN3CCN(C(=O)COc4cccc5c4C(=O)N(C4CCC(=O)NC4=O)C5=O)CC3)c12]

Tert-butyl 1-{2-[4-(2-{[2-(2,6-dioxopiperidin-3-yl)-1,3-dioxo-2,3-dihydro-1H-isoindol-4-yl]oxy}acetyl)piperazin-1-yl]ethyl}-6-fluoro-3-{3-[(6-fluoronaphthalen-1-yl)oxy]propyl}-7-(1,3,5-trimethyl-1H-pyrazol-4-yl)-1H-indole-2-carboxylate (44.0 mg, crude) was dissolved in dry DCM (0.35 mL) and TFA (0.347 mL, 4.526 mmol) was added. Reaction was stirred for 13 h at room temperature. After full consumption of the starting material was confirmed with LCMS, the solution was concentrated and resulting material dried under reduced pressure. Crude product was purified twice with preparative TLC (20% MeOH in DCM and 10% to 20% MeOH in DCM). Reaction product was washed from silica gel with eluent solution, filtered and dried under reduced pressure to give 1-{2-[4-(2-{[2-(2,6-dioxopiperidin-3-yl)-1,3-dioxo-2,3-dihydro-1H-isoindol-4-yl]oxy}acetyl)piperazin-1-yl]ethyl}-6-fluoro-3-{3-[(6-fluoronaphthalen-1-yl)oxy]propyl}-7-(1,3,5-trimethyl-1H-pyrazol-4-yl)-1H-indole-2-carboxylic acid (3.1 mg, 0.003 mmol, 6% yield over two steps) as off white solid.

LCMS (ESI+): m/z 916.0 [M+H]⁺

¹H NMR (500 MHz, DMSO) δ 13.26 (bs, 1H), 11.11 (s, 1H), 8.27 (dd, J=9.3, 5.9 Hz, 1H), 7.77 (dd, J=8.6, 7.2 Hz, 2H), 7.67 (dd, J=10.4, 2.6 Hz, 1H), 7.46 (dd, J=5.3, 2.0 Hz, 3H), 7.39 (td, J=8.9, 2.6 Hz, 1H), 7.31 (d, J=8.6 Hz, 1H), 7.09-6.98 (m, 1H), 6.90 (dd, J=5.4, 3.3 Hz, 1H), 5.20-5.06 (m, 3H), 4.47-4.32 (m, 1H), 4.41-4.18 (m, 3H), 3.77 (s, 3H), 3.54-3.43 (m, 1H), 3.41 (s, 1H), 3.28-3.26 (m, 2H), 2.90 (ddd, J=16.9, 13.7, 5.4 Hz, 1H), 2.65-2.60 (m, 1H), 2.60-2.57 (m, 1H), 2.57-2.55 (m, 1H), 2.28-2.18 (m, 2H), 2.18-1.96 (m, 11H), 1.93 (s, 3H).

### Example 21: 1-(2-(4-(2-((2-(2,6-dioxopiperidin-3-yl)-1,3-dioxoisoindolin-4-yl)oxy)acetyl)piperazin-1-yl)ethyl)-6-methyl-3-(3-(naphthalen-1-yloxy)propyl)-7-(1,3,5-trimethyl-1H-pyrazol-4-yl)-1H-indole-2-carboxylic acid (217)

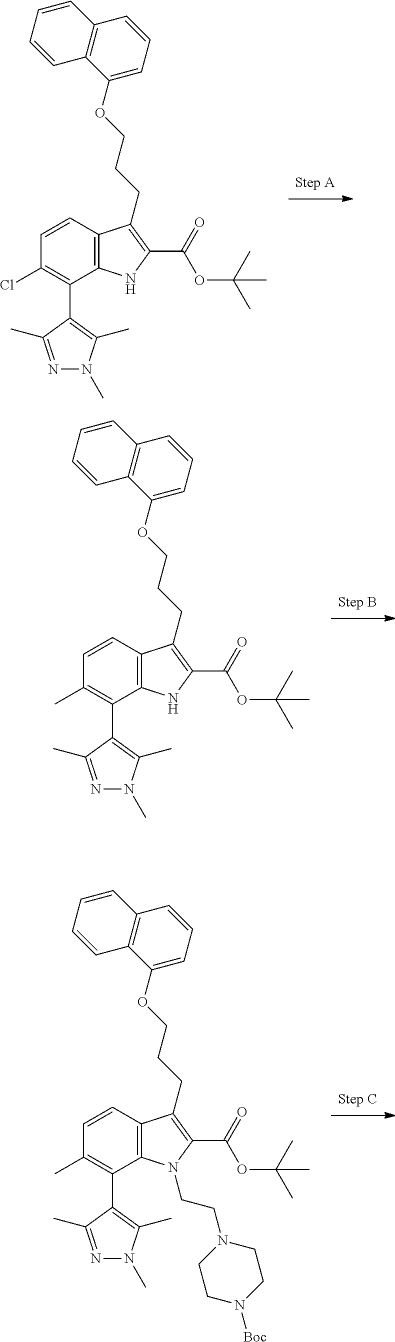

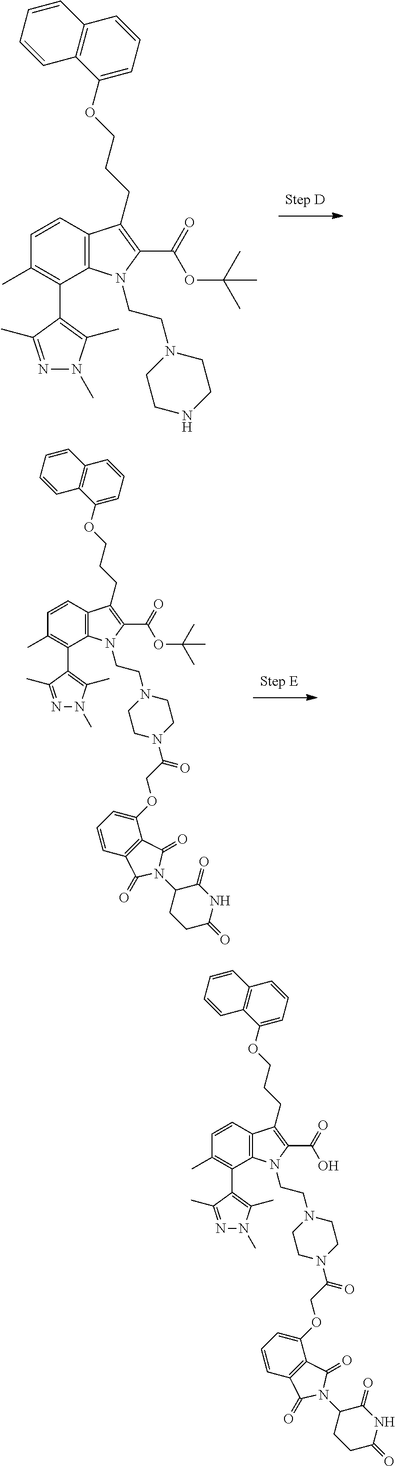

### Step A

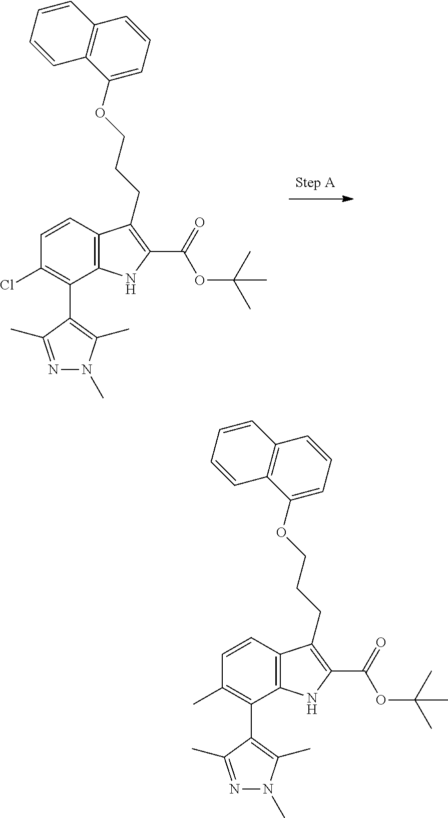 [SMILES: Cc1nn(C)c(C)c1-c1c(Cl)ccc2c(CCCOc3cccc4ccccc34)c(C(=O)OC(C)(C)C)[nH]c12 ; Cc1ccc2c(CCCOc3cccc4ccccc34)c(C(=O)OC(C)(C)C)[nH]c2c1-c1c(C)nn(C)c1C]

To a stirred solution of tert-butyl 6-chloro-3-(3-(naphthalen-1-yloxy)propyl)-7-(1,3,5-trimethyl-1H-pyrazol-4-yl)-1H-indole-2-carboxylate (2 g, 3.683 mmol) in dioxane (20 mL) were added methyl boronic acid (761 mg, 12.891 mmol) and potassium phosphate (2.343 g, 11.05 mmol), 2-dicyclohexylphosphino-2′,6′-dimethoxybiphenyl (SPhos, 453 mg, 1.105 mmol). The mixture was deoxygenated with argon and to it was added Pd(OAc)₂ (0.124 g, 0.552 mmol) under argon atmosphere. Then the reaction mixture was heated under reflux for 16 h. After complete consumption of the starting material (monitored by TLC and LCMS) the reaction mixture was filtered through celite pad and the solvents were evaporated under reduced pressure. It was then diluted with EtOAc and washed successively with water and brine. The organic layer was dried over sodium sulphate and concentrated under reduced pressure to get the crude compound, which was then purified by column chromatography (SiO₂, 30% EtOAc in DCM) to get tert-butyl 6-methyl-3-(3-(naphthalen-1-yloxy)propyl)-7-(1,3,5-trimethyl-1H-pyrazol-4-yl)-1H-indole-2-carboxylate (1.6 g, 3.05 mmol, 82.8%) as brown solid.

LCMS (ESI+): m/z 523.7 [M+H]⁺

### Step B

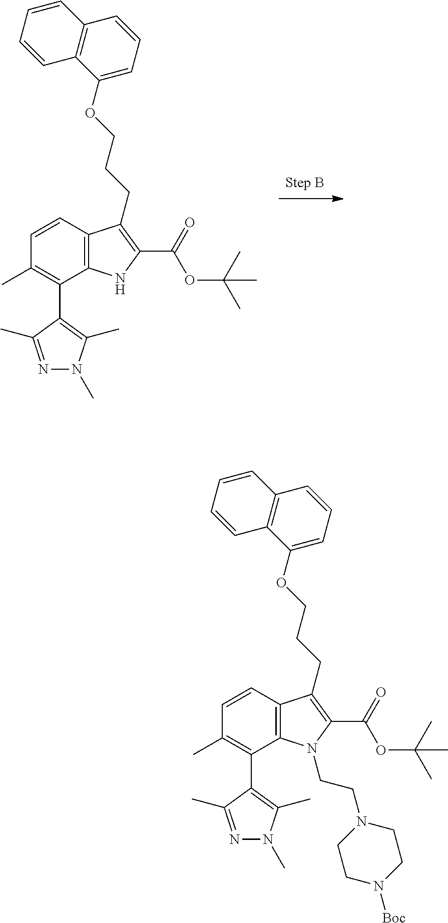 [SMILES: Cc1ccc2c(CCCOc3cccc4ccccc34)c(C(=O)OC(C)(C)C)[nH]c2c1-c1c(C)nn(C)c1C ; Cc1ccc2c(CCCOc3cccc4ccccc34)c(C(=O)OC(C)(C)C)n(CCN3CCN(C(=O)OC(C)(C)C)CC3)c2c1-c1c(C)nn(C)c1C]

To a well stirred solution of tert-butyl 6-methyl-3-(3-(naphthalen-1-yloxy)propyl)-7-(1,3,5-trimethyl-1H-pyrazol-4-yl)-1H-indole-2-carboxylate (1.6 g, 3.05 mmol) in DMF (15 mL) was added tert-butyl 4-(2-chloroethyl)piperazine-1-carboxylate (1.5 g, 6.119 mmol) followed by cesium carbonate (4.9 g, 15.296 mmol) in DMF and the mixture was allowed to stir at 90° C. for 16 h under nitrogen. After complete consumption of the starting material (monitored by TLC and LCMS) the reaction mixture was diluted with EtOAc, washed successively with water and brine, the organic layer was dried over sodium sulphate and evaporated under reduced pressure to get the crude compound, which was then purified by column chromatography (SiO₂, 30% EtOAc in DCM) to get tert-butyl 1-(2-(4-(tert-butoxycarbonyl)piperazin-1-yl)ethyl)-6-methyl-3-(3-(naphthalen-1-yloxy)propyl)-7-(1,3,5-trimethyl-1H-pyrazol-4-yl)-1H-indole-2-carboxylate (1.2 g, 1.63 mmol, 53.4%) as white solid.

LCMS (ESI+): m/z 737.4 [M+H]⁺

### Step C

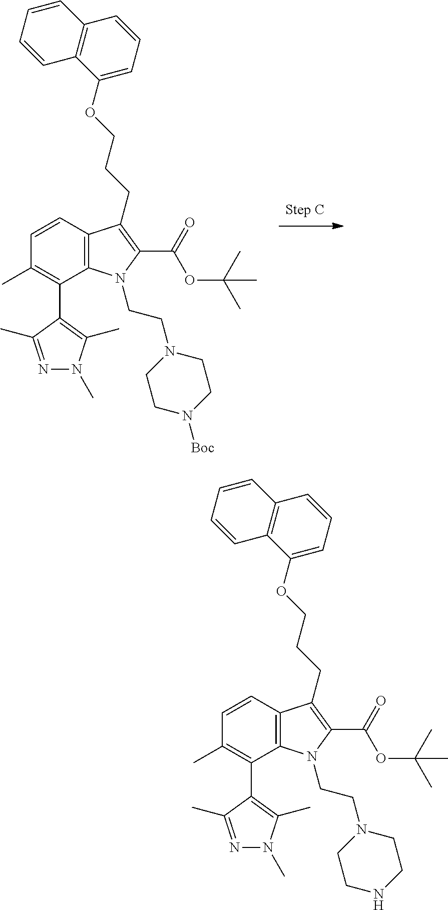 [SMILES: Cc1ccc2c(CCCOc3cccc4ccccc34)c(C(=O)OC(C)(C)C)n(CCN3CCN(C(=O)OC(C)(C)C)CC3)c2c1-c1c(C)nn(C)c1C ; Cc1ccc2c(CCCOc3cccc4ccccc34)c(C(=O)OC(C)(C)C)n(CCN3CCNCC3)c2c1-c1c(C)nn(C)c1C]

Tert-butyl 1-(2-(4-(tert-butoxycarbonyl)piperazin-1-yl)ethyl)-6-methyl-3-(3-(naphthalen-1-yloxy)propyl)-7-(1,3,5-trimethyl-1H-pyrazol-4-yl)-1H-indole-2-carboxylate (800 mg, 1.088 mmol) was dissolved in DCM (30 mL) and 4M HCl in dioxane (20 mL) was added drop-wise at 0° C. The mixture was stirred for 2 h under nitrogen at that temperature. Upon indication by LCMS full conversion of the starting material, the reaction mixture was quenched by drop wise addition of cold 1M NaOH solution at 0° C. to reach the pH˜7 to 8. The aqueous layer was extracted 2-3 times with DCM. The combined organics were dried over Na₂SO₄ and concentrated under vacuum to get the crude compound which was then purified by column chromatography (amine SiO₂, 70% EtOAc in DCM) to get tert-butyl 6-methyl-3-(3-(naphthalen-1-yloxy)propyl)-1-(2-(piperazin-1-yl)ethyl)-7-(1,3,5-trimethyl-1H-pyrazol-4-yl)-1H-indole-2-carboxylate (440 mg, 0.692 mmol, 63.58%) as white solid.

LCMS (ESI+): m/z 636.5 [M+H]⁺

### Step D

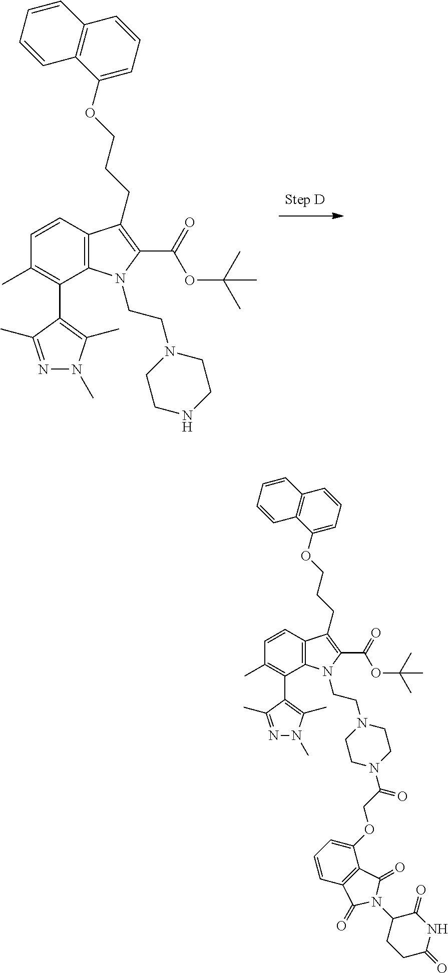 [SMILES: Cc1ccc2c(CCCOc3cccc4ccccc34)c(C(=O)OC(C)(C)C)n(CCN3CCNCC3)c2c1-c1c(C)nn(C)c1C ; Cc1ccc2c(CCCOc3cccc4ccccc34)c(C(=O)OC(C)(C)C)n(CCN3CCN(C(=O)COc4cccc5c4C(=O)N(C4CCC(=O)NC4=O)C5=O)CC3)c2c1-c1c(C)nn(C)c1C]

To a well stirred solution of 2-{[2-(2,6-dioxopiperidin-3-yl)-1,3-dioxo-2,3-dihydro-1H-isoindol-4-yl]oxy}acetic acid (30.4 mg, 0.091 mmol) and tert-butyl 6-methyl-3-(3-(naphthalen-1-yloxy)propyl)-1-(2-(piperazin-1-yl)ethyl)-7-(1,3,5-trimethyl-1H-pyrazol-4-yl)-1H-indole-2-carboxylate (48.4 mg, 0.076 mmol) in DMF (0.762 mL) was added DIPEA (0.040 mL, 0.229 mmol) and HATU (43.5 mg, 0.114 mmol) and the mixture was allowed to stir under nitrogen for 2 h. After complete consumption of the starting material (monitored by TLC and LCMS), the reaction mixture was diluted with EtOAc and washed successively with cold water and brine. Organic layer was dried over Na₂SO₄ and evaporated under reduced pressure. Crude mixture was purified by flash chromatography (SiO₂ DCM:MeOH, 0-10% MeOH) to give tert-butyl 1-(2-(4-(2-((2-(2,6-dioxopiperidin-3-yl)-1,3-dioxoisoindolin-4-yl)oxy)acetyl)piperazin-1-yl)ethyl)-6-methyl-3-(3-(naphthalen-1-yloxy)propyl)-7-(1,3,5-trimethyl-1H-pyrazol-4-yl)-1H-indole-2-carboxylate (39.0 mg, 0.036 mmol, 47.8%) as yellow gum.

LCMS (ESI+): m/z 950.4 [M+H]⁺

### Step E

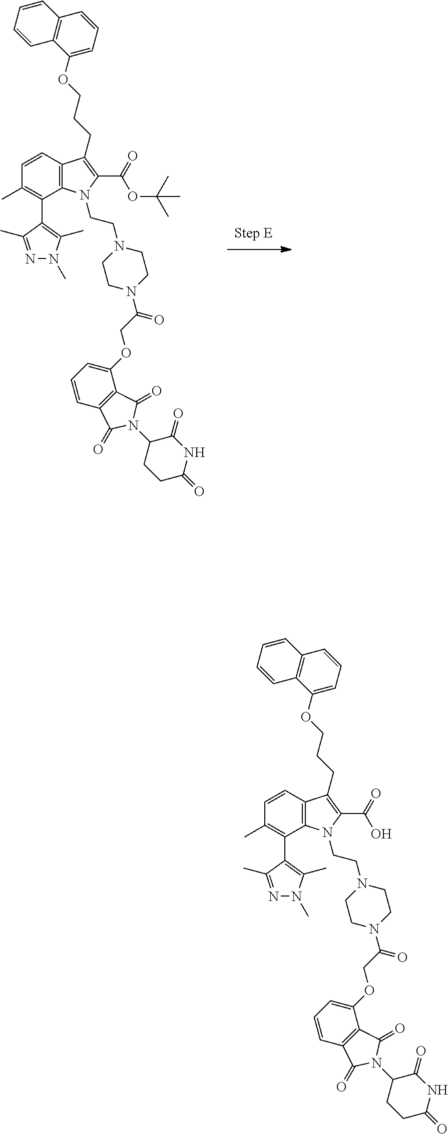 [SMILES: Cc1ccc2c(CCCOc3cccc4ccccc34)c(C(=O)OC(C)(C)C)n(CCN3CCN(C(=O)COc4cccc5c4C(=O)N(C4CCC(=O)NC4=O)C5=O)CC3)c2c1-c1c(C)nn(C)c1C ; Cc1ccc2c(CCCOc3cccc4ccccc34)c(C(=O)O)n(CCN3CCN(C(=O)COc4cccc5c4C(=O)N(C4CCC(=O)NC4=O)C5=O)CC3)c2c1-c1c(C)nn(C)c1C]

Tert-butyl 1-(2-(4-(2-((2-(2,6-dioxopiperidin-3-yl)-1,3-dioxoisoindolin-4-yl)oxy)acetyl)piperazin-1-yl)ethyl)-6-methyl-3-(3-(naphthalen-1-yloxy)propyl)-7-(1,3,5-trimethyl-1H-pyrazol-4-yl)-1H-indole-2-carboxylate (39.0 mg, 0.041 mmol) was placed in a capped vial, dissolved in DCM and then TFA (0.629 mL, 8.210 mmol) was added. Reaction was stirred overnight at RT. Solvents were removed under reduced pressure, and crude product was purified using preparative HPLC (H₂O:MeCN+0.1% FA) to afford 1-(2-(4-(2-((2-(2,6-dioxopiperidin-3-yl)-1,3-dioxoisoindolin-4-yl)oxy)acetyl)piperazin-1-yl)ethyl)-6-methyl-3-(3-(naphthalen-1-yloxy)propyl)-7-(1,3,5-trimethyl-1H-pyrazol-4-yl)-1H-indole-2-carboxylic acid (16.6 mg, 0.019 mmol, 45.2%) as yellow solid.

LCMS (ESI+): m/z 894.2 [M+H]⁺

¹H NMR (500 MHz, DMSO) δ 13.14 (s, 1H), 11.09 (s, 1H), 8.28-8.21 (m, 1H), 7.89-7.82 (m, 1H), 7.75 (dd, J=8.6, 7.2 Hz, 1H), 7.61 (d, J=8.1 Hz, 1H), 7.56-7.48 (m, 2H), 7.45 (t, 2H), 7.39 (t, J=7.9 Hz, 1H), 7.29 (d, J=8.6 Hz, 1H), 7.02 (d, J=8.2 Hz, 1H), 6.94-6.86 (m, 1H), 5.15-5.05 (m, 3H), 4.29 (dt, J=13.8, 7.0 Hz, 1H), 4.21 (t, J=6.2 Hz, 2H), 4.18-4.10 (m, 1H), 3.75 (s, 3H), 3.36-3.33 (m, 3H), 3.28-3.24 (m, 3H), 2.88 (ddd, J=16.8, 13.8, 5.4 Hz, 1H), 2.62-2.52 (m, 2H), 2.21 (p, J=6.4 Hz, 2H), 2.16-2.02 (m, 7H), 2.01 (s, 3H), 1.98 (s, 3H), 1.85 (s, 3H).

### Example 22: 3-(3-(4-chloro-3,5-dimethylphenoxy)propyl)-7-(1-(3-(2-(2-((2-(2,6-dioxopiperidin-3-yl)-1,3-dioxoisoindolin-4-yl)amino)ethoxy)ethoxy)propyl)-3,5-dimethyl-1H-pyrazol-4-yl)-1-methyl-1H-indole-2-carboxylic acid (227)

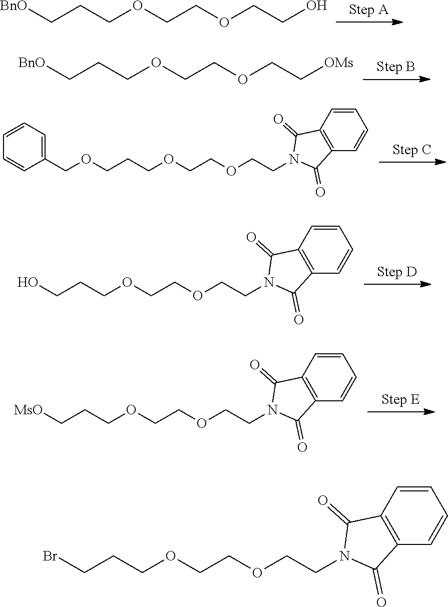

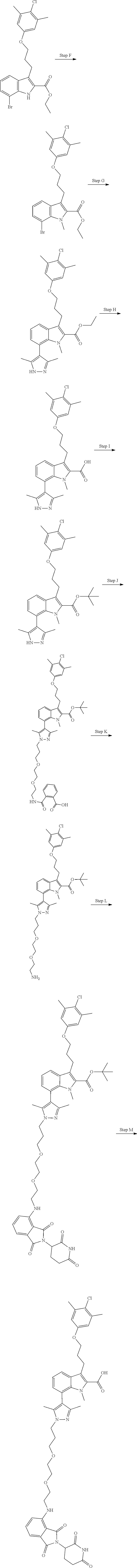

### Step A

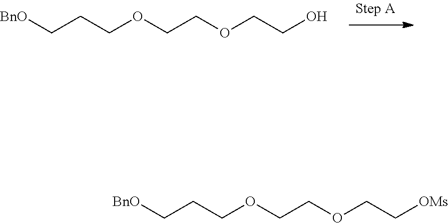 [SMILES: OCCOCCOCCCOCc1ccccc1 ; CS(=O)(=O)OCCOCCOCCCOCc1ccccc1]

To a stirred solution of 2-(2-(3-(benzyloxy)propoxy)ethoxy)ethan-1-ol (5.8 g, 22.8 mmol) in DCM (100 mL) was added MsCl (10 mL, 68.4 mmol), followed by triethyl amine (6.4 mL, 45.6 mmol) at 0° C. under nitrogen and the reaction mixture was allowed to stir at room temperature for 2 h. After complete consumption of the starting material the reaction mixture was diluted with EtOAc, washed successively with water and brine, the organic layer was dried over sodium sulphate and evaporated under reduced pressure to get the crude compound, which was then purified by column chromatography (SiO₂, 50% EtOAc in hexane) to get 2-(2-(3-(benzyloxy)propoxy)ethoxy)ethyl methanesulfonate (6.7 g, 20.2 mmol, 88%) as colorless liquid.

LCMS (ESI+): m/z 333.0 [M+H]⁺

### Step B

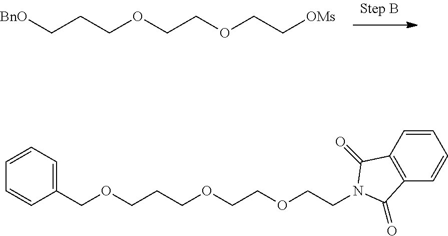 [SMILES: CS(=O)(=O)OCCOCCOCCCOCc1ccccc1 ; O=C1c2ccccc2C(=O)N1CCOCCOCCCOCc1ccccc1]

To a stirred solution of 2-(2-(3-(benzyloxy)propoxy)ethoxy)ethyl methanesulfonate (6.2 g, 18.6 mmol) in DMF (60 mL) was added lithium bromide (4.8 g, 56 mmol) and the reaction mixture was allowed to stir at 100° C. under nitrogen for 4 h. After complete consumption of the starting material reaction mixture was cooled to room temperature, to it was added potassium phthalimide (6.9 g, 37.3 mmol) and reaction mixture was stirred for another 16 h at 100° C. under nitrogen. After complete consumption of the starting material the reaction mixture was diluted with EtOAc and washed successively with water and brine. The organic layer was dried over Na₂SO₄ and evaporated under reduced pressure to get the crude compound, which was then purified by column chromatography (SiO₂, 30% EtOAc in hexane) to get 2-(2-(2-(3-(benzyloxy)propoxy)ethoxy)ethyl)isoindoline-1,3-dione (3.2 g, 8.35 mmol, 45%) as yellow dense liquid.

LCMS (ESI+): m/z 384.2 [M+H]⁺

### Step C

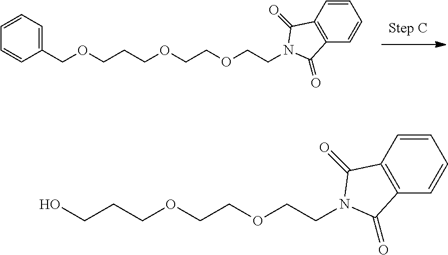 [SMILES: O=C1c2ccccc2C(=O)N1CCOCCOCCCOCc1ccccc1 ; O=C1c2ccccc2C(=O)N1CCOCCOCCCO]

To a stirred solution of 2-(2-(2-(3-(benzyloxy)propoxy)ethoxy)ethyl)isoindoline-1,3-dione (3.5 g, 9.13 mmol) in DCM (50 ml) was added boron trichloride (1M solution in DCM, 28 mL, 28 mmol) drop wise at −78° C. under nitrogen. The reaction mixture was allowed to stir at same temp for 1 h. After complete consumption of the starting material the reaction mixture was poured into ice, extracted with EtOAc and washed successively with water and brine. The organic layer was dried over sodium sulphate and evaporated under reduced pressure to get the crude compound, which was then purified by column chromatography (SiO₂, 80% EtOAc in DCM) to get 2-(2-(2-(3-hydroxypropoxy)ethoxy)ethyl)isoindoline-1,3-dione (2.1 g, 7.16 mmol, 78%) as colorless dense liquid.

LCMS (ESI+): m/z 293.8 [M+H]⁺

### Step D

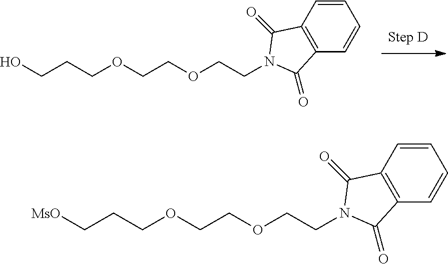 [SMILES: O=C1c2ccccc2C(=O)N1CCOCCOCCCO ; CS(=O)(=O)OCCCOCCOCCN1C(=O)c2ccccc2C1=O]

To a stirred solution of 2-(2-(2-(3-hydroxypropoxy)ethoxy)ethyl)isoindoline-1,3-dione (2.5 g, 8.5 mmol) in DCM (60 mL) was added mesyl chloride (2.5 mL, 17.06 mmol) followed by Et₃N (3.5 mL, 25.6 mmol) at 0° C. under nitrogen and the reaction mixture was allowed to stir at room temperature for 2 h. After complete consumption of the starting material the reaction mixture was diluted with EtOAc and washed successively with water and brine. The organic layer was dried over sodium sulphate and evaporated under reduced pressure to get the crude compound, which was then purified by column chromatography (SiO₂, 40% EtOAc in hexane) to get 3-(2-(2-(1,3-dioxoisoindolin-2-yl)ethoxy)ethoxy)propyl methanesulfonate (3 g, 8.08 mmol, 95%) as colorless liquid.

LCMS (ESI+): m/z 372.2 [M+H]⁺

### Step E

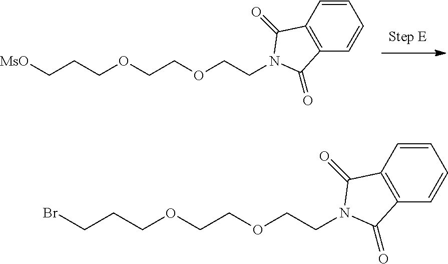 [SMILES: CS(=O)(=O)OCCCOCCOCCN1C(=O)c2ccccc2C1=O ; O=C1c2ccccc2C(=O)N1CCOCCOCCCBr]

To a stirred solution of 3-(2-(2-(1,3-dioxoisoindolin-2-yl)ethoxy)ethoxy)propyl methanesulfonate (3 g, 8.08 mmol) in DMF (40 mL) was added lithium bromide (3.5 g, 40.43 mmol) and the reaction mixture was allowed to stir at room temperature for 16 h under nitrogen. After complete consumption of the starting material (monitored by TLC and LCMS) the reaction mixture was diluted with EtOAc, and washed successively with water and brine. The organic layer was dried over Na₂SO₄ and evaporated under reduced pressure to get the crude compound, which was then purified by column chromatography (SiO₂, 30% EtOAc in hexane) to get 2-(2-(2-(3-bromopropoxy)ethoxy)ethyl)isoindoline-1,3-dione (2.5 g, 7.02 mmol, 87%) as colorless dense liquid.

LCMS (ESI+): m/z 356.2 [M+H]⁺

### Step F

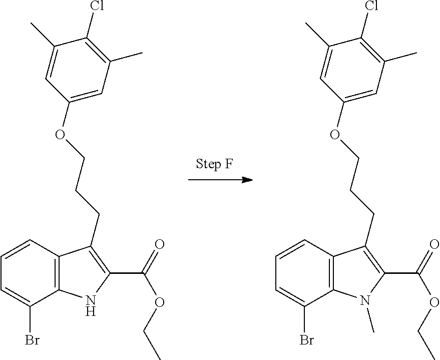 [SMILES: CCOC(=O)c1[nH]c2c(Br)cccc2c1CCCOc1cc(C)c(Cl)c(C)c1 ; CCOC(=O)c1c(CCCOc2cc(C)c(Cl)c(C)c2)c2cccc(Br)c2n1C]

To a well stirred solution of ethyl 7-bromo-3-(3-(4-chloro-3,5-dimethylphenoxy)propyl)-1H-indole-2-carboxylate (10 g, 21.55 mmol) in DMF (100 mL) was added potassium carbonate (11.9 g, 86.2 mmol) followed by methyl iodide (2.8 mL, 43.1 mmol) and the mixture was allowed to stir at 90° C. for 16 h under nitrogen. After complete consumption of the starting material the reaction mixture was diluted with EtOAc, washed successively with water and brine, the organic layer was dried over Na₂SO₄ and evaporated under reduced pressure to get the crude compound, which was then purified by column chromatography (SiO₂, EtOAc:hexane, 10-20%) to get ethyl 7-bromo-3-(3-(4-chloro-3,5-dimethylphenoxy)propyl)-1-methyl-1H-indole-2-carboxylate (10 g, 21 mmol, 97%) as off white solid.

LCMS (ESI+): m/z 477.7[M+H]⁺

### Step G

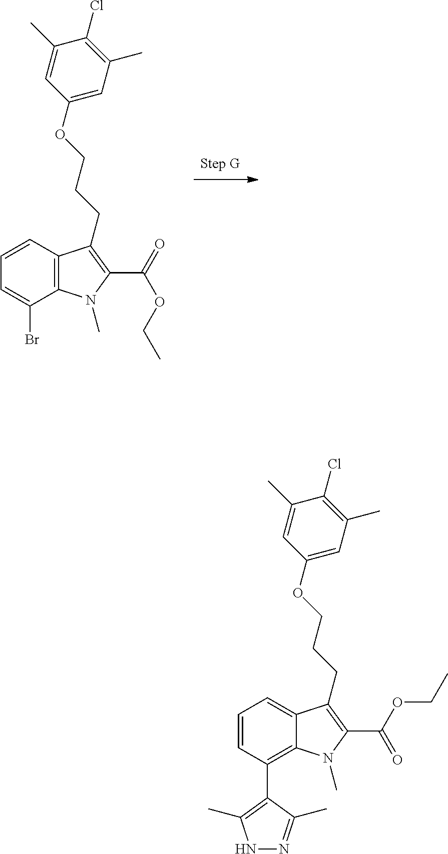 [SMILES: CCOC(=O)c1c(CCCOc2cc(C)c(Cl)c(C)c2)c2cccc(Br)c2n1C ; CCOC(=O)c1c(CCCOc2cc(C)c(Cl)c(C)c2)c2cccc(-c3c(C)n[nH]c3C)c2n1C]

To a stirred solution of ethyl 7-bromo-3-(3-(4-chloro-3,5-dimethylphenoxy)propyl)-1-methyl-1H-indole-2-carboxylate (5 g, 10.46 mmol) in dioxane (150 mL) and water (30 mL) were added 3,5-dimethyl-1-(tetrahydro-2H-pyran-2-yl)-4-(4,4,5,5-tetramethyl-1,3,2-dioxaborolan-2-yl)-1H-pyrazole (7 g, 31.4 mmol) and K₂CO₃ (5.8 g, 41.84 mmol). The mixture was deoxygenated with argon and to it was added Pd(dppf)Cl₂ (1.14 g, 1.57 mmol) under argon atmosphere. Then the reaction mixture was heated under reflux for 16 h. After complete consumption of the starting material the reaction mixture was filtered through celite pad and the solvents were evaporated under reduced pressure get the crude material. It was then diluted with EtOAc, washed successively with water and brine, the organic layer was dried over Na₂SO₄ and evaporated under reduced pressure to get the crude compound, which was then purified by column chromatography (SiO₂, 5% MeOH in DCM) to get ethyl 3-(3-(4-chloro-3,5-dimethylphenoxy)propyl)-7-(3,5-dimethyl-1H-pyrazol-4-yl)-1-methyl-1H-indole-2-carboxylate (3 g, 6.05 mmol, 58%) as brown gummy solid.

LCMS (ESI+): m/z 494.2 [M+H]⁺

### Step H

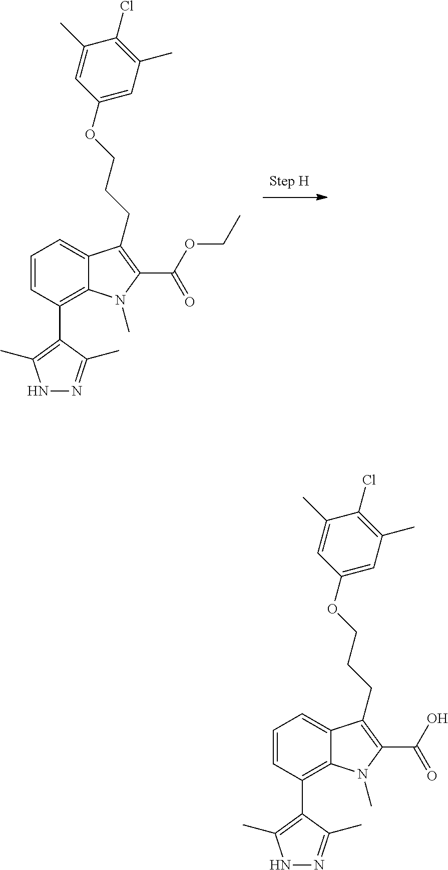 [SMILES: CCOC(=O)c1c(CCCOc2cc(C)c(Cl)c(C)c2)c2cccc(-c3c(C)n[nH]c3C)c2n1C ; Cc1cc(OCCCc2c(C(=O)O)n(C)c3c(-c4c(C)n[nH]c4C)cccc23)cc(C)c1Cl]

3-(3-(4-Chloro-3,5-dimethylphenoxy)propyl)-7-(3,5-dimethyl-1H-pyrazol-4-yl)-1-methyl-1H-indole-2-carboxylate (2 g, 4.06 mmol) was dissolved in EtOH (40 mL) and a solution of NaOH (0.57 g, 14.2 mmol) in water (8 mL) was added to it. The mixture was heated under reflux for 3 h. After complete consumption of the starting material the reaction mixture was cooled down to room temperature, solvents were evaporated under reduced pressure to get the crude reaction mixture. It was then diluted with water, washed with EtOAc. Aqueous layer was carefully acidified using 1M HCl to pH=3, extracted with DCM, dried over Na₂SO₄, filtered, and concentrated in vacuo to afford 3-(3-(4-chloro-3,5-dimethylphenoxy)propyl)-7-(3,5-dimethyl-1H-pyrazol-4-yl)-1-methyl-1H-indole-2-carboxylic acid (2 g, crude) as dark brown gummy solid.

LCMS (ESI+): m/z 466.5 [M+H]⁺

### Step I

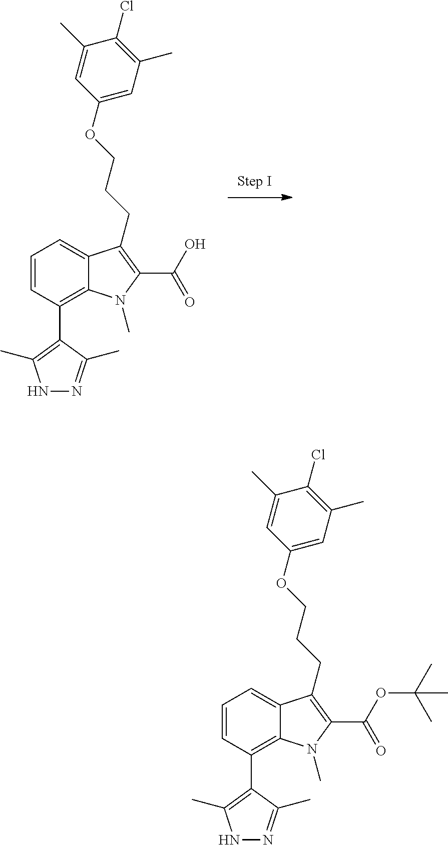 [SMILES: Cc1cc(OCCCc2c(C(=O)O)n(C)c3c(-c4c(C)n[nH]c4C)cccc23)cc(C)c1Cl ; Cc1cc(OCCCc2c(C(=O)OC(C)(C)C)n(C)c3c(-c4c(C)n[nH]c4C)cccc23)cc(C)c1Cl]

3-(3-(4-Chloro-3,5-dimethylphenoxy)propyl)-7-(3,5-dimethyl-1H-pyrazol-4-yl)-1-methyl-1H-indole-2-carboxylic acid (2 g, crude) was suspended in toluene (20 mL) and the mixture was heated to reflux under nitrogen. N,N-dimethylformamide di-tert-butyl acetal (3.5 mL, 17.2 mmol) was added drop wise to the refluxing mixture. Refluxing was continued for an additional 16 h under nitrogen. After 16 h another 1.7 mL (8.6 mmol) of N,N-dimethylformamide di-tert-butyl acetal was added to it and the reaction was continued for another 24 h. Reaction mixture was then diluted with EtOAc, washed successively with NaHCO₃ (saturated), water and brine. Organic layer was dried over Na₂SO₄ and concentrated under reduced pressure to get the crude compound, which was then purified by column chromatography (SiO₂, 2% MeOH in DCM) to get tert-butyl 3-(3-(4-chloro-3,5-dimethylphenoxy)propyl)-7-(3,5-dimethyl-1H-pyrazol-4-yl)-1-methyl-1H-indole-2-carboxylate (1.4 g, 2.68 mmol, 62%) as brown solid.

LCMS (ESI+): m/z 522.2 [M+H]⁺

### Step J

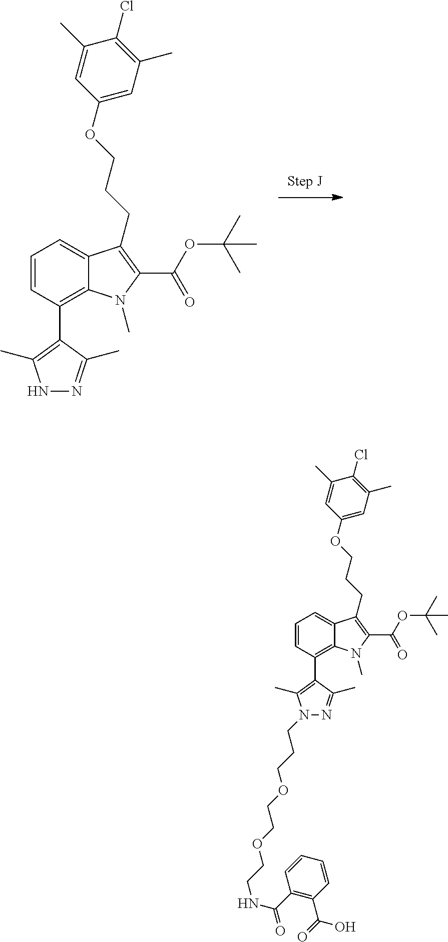 [SMILES: Cc1cc(OCCCc2c(C(=O)OC(C)(C)C)n(C)c3c(-c4c(C)n[nH]c4C)cccc23)cc(C)c1Cl ; Cc1cc(OCCCc2c(C(=O)OC(C)(C)C)n(C)c3c(-c4c(C)nn(CCCOCCOCCNC(=O)c5ccccc5C(=O)O)c4C)cccc23)cc(C)c1Cl]

To a stirred solution of tert-butyl 3-(3-(4-chloro-3,5-dimethylphenoxy)propyl)-7-(3,5-dimethyl-1H-pyrazol-4-yl)-1-methyl-1H-indole-2-carboxylate (600 mg, 1.15 mmol) in DMF (15 mL) at 0° C. was added sodium hydride (55.3 mg, 2.3 mmol) portion-wise under nitrogen and the reaction mixture was allowed to stir at the same temperature for 1 h, after that to the reaction mixture was added 2-(2-(2-(3-bromopropoxy)ethoxy)ethyl)isoindoline-1,3-dione (1.3 g, 3.45 mmol) dissolved in DMF (5 mL) at 0° C. and the reaction mixture was allowed to stir at room temperature for 16 h under nitrogen. After complete consumption of the starting material (monitored by TLC and LCMS) the reaction mixture was again cooled to 0° C. and quenched the excess of NaH with ammonium chloride solution. The volatiles were evaporated under reduced pressure to get the crude 2-((2-(2-(3-(4-(2-(tert-butoxycarbonyl)-3-(3-(4-chloro-3,5-dimethylphenoxy)propyl)-1-methyl-1H-indol-7-yl)-3,5-dimethyl-1H-pyrazol-1-yl)propoxy)ethoxy)ethyl)carbamoyl)benzoic acid which was used for the next step without further purification.

### Step K

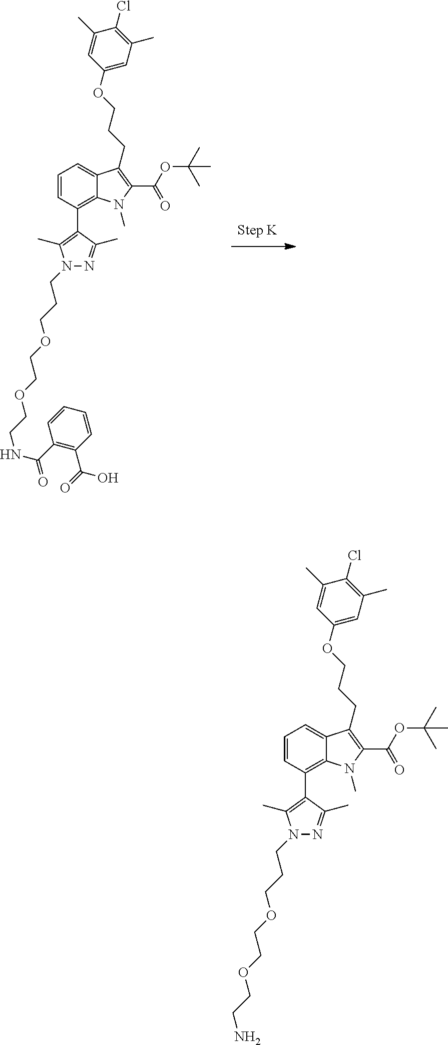 [SMILES: Cc1cc(OCCCc2c(C(=O)OC(C)(C)C)n(C)c3c(-c4c(C)nn(CCCOCCOCCNC(=O)c5ccccc5C(=O)O)c4C)cccc23)cc(C)c1Cl ; Cc1cc(OCCCc2c(C(=O)OC(C)(C)C)n(C)c3c(-c4c(C)nn(CCCOCCOCCN)c4C)cccc23)cc(C)c1Cl]

A mixture of 2-((2-(2-(3-(4-(2-(tert-butoxycarbonyl)-3-(3-(4-chloro-3,5-dimethylphenoxy)propyl)-1-methyl-1H-indol-7-yl)-3,5-dimethyl-1H-pyrazol-1-yl)propoxy)ethoxy)ethyl)carbamoyl)benzoic acid and hydrazine hydrate (1.63 mL, 33 mmol) in 30 mL of dry tert-butanol was refluxed for 16 h at 140° C. under nitrogen. Then it was allowed to cool down to room temperature. Volatiles were evaporated under reduced pressure, the resulting residue was dissolved in DCM and washed successively with water and brine. The organic layer was dried over MgSO₄, filtered and evaporated under reduced pressure to get the crude compound which was purified by column chromatography (amine SiO₂, 2% MeOH in DCM) tert-butyl 7-(1-(3-(2-(2-aminoethoxy)ethoxy)propyl)-3,5-dimethyl-1H-pyrazol-4-yl)-3-(3-(4-chloro-3,5-dimethylphenoxy)propyl)-1-methyl-1H-indole-2-carboxylate (150 mg, 0.224 mmol, 19% over 2 steps) as brown liquid.

LCMS (ESI+): m/z 667.5 [M+H]⁺

### Step L

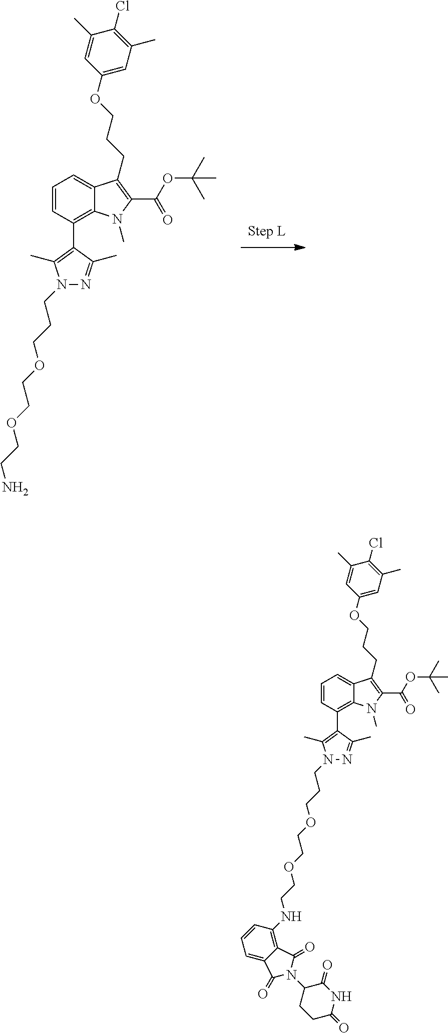 [SMILES: Cc1cc(OCCCc2c(C(=O)OC(C)(C)C)n(C)c3c(-c4c(C)nn(CCCOCCOCCN)c4C)cccc23)cc(C)c1Cl ; Cc1cc(OCCCc2c(C(=O)OC(C)(C)C)n(C)c3c(-c4c(C)nn(CCCOCCOCCNc5cccc6c5C(=O)N(C5CCC(=O)NC5=O)C6=O)c4C)cccc23)cc(C)c1Cl]

To a well stirred solution of tert-butyl 7-(1-(3-(2-(2-aminoethoxy)ethoxy)propyl)-3,5-dimethyl-1H-pyrazol-4-yl)-3-(3-(4-chloro-3,5-dimethylphenoxy)propyl)-1-methyl-1H-indole-2-carboxylate (650 mg, 0.975 mmol) in DMSO (15 mL) was added 2-(2,6-dioxopiperidin-3-yl)-4-fluoroisoindoline-1,3-dione (403 mg, 1.46 mmol), followed by DIPEA (339 μL, 1.95 mmol) and the reaction mixture was allowed to stir in 90° C. for 2 h under nitrogen. After complete consumption of the starting material the reaction mixture was diluted with EtOAc and washed successively with water and brine. The organic layer was dried over Na₂SO₄ and evaporated under reduced pressure to get the crude compound, which was then purified by column chromatography (SiO₂, 80% EtOAc in DCM) to get tert-butyl 3-(3-(4-chloro-3,5-dimethylphenoxy)propyl)-7-(1-(3-(2-(2-((2-(2,6-dioxopiperidin-3-yl)-1,3-dioxoisoindolin-4-yl)amino)ethoxy)ethoxy)propyl)-3,5-dimethyl-1H-pyrazol-4-yl)-1-methyl-1H-indole-2-carboxylate (230 mg, 0.25 mmol, 25%) as yellow solid.

LCMS (ESI+): m/z 923.9 [M+H]⁺

### Step M

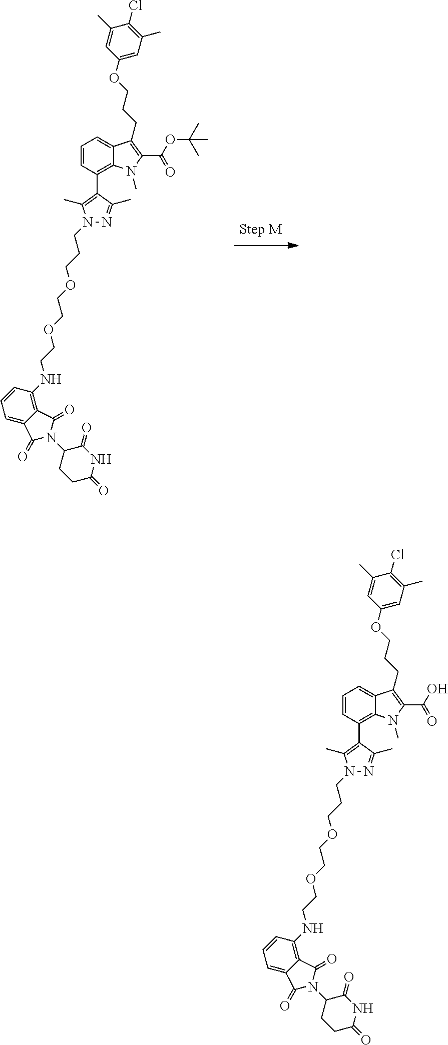 [SMILES: Cc1cc(OCCCc2c(C(=O)OC(C)(C)C)n(C)c3c(-c4c(C)nn(CCCOCCOCCNc5cccc6c5C(=O)N(C5CCC(=O)NC5=O)C6=O)c4C)cccc23)cc(C)c1Cl ; Cc1cc(OCCCc2c(C(=O)O)n(C)c3c(-c4c(C)nn(CCCOCCOCCNc5cccc6c5C(=O)N(C5CCC(=O)NC5=O)C6=O)c4C)cccc23)cc(C)c1Cl]

To a well stirred solution of tert-butyl 3-(3-(4-chloro-3,5-dimethylphenoxy)propyl)-7-(1-(3-(2-(2-((2-(2,6-dioxopiperidin-3-yl)-1,3-dioxoisoindolin-4-yl)amino)ethoxy)ethoxy)propyl)-3,5-dimethyl-1H-pyrazol-4-yl)-1-methyl-1H-indole-2-carboxylate (120 mg, 0.13 mmol) in dioxane (3 mL) was added 5 mL of 4M HCl in dioxane and the reaction mixture was allowed to stir at room temperature for 28 h. After complete consumption of the starting material the reaction mixture was evaporated under reduced pressure to get the crude compound, which was then purified by preparative HPLC (H₂O:MeCN+0.1% HCl) to get 3-(3-(4-chloro-3,5-dimethylphenoxy)propyl)-7-(1-(3-(2-(2-((2-(2,6-dioxopiperidin-3-yl)-1,3-dioxoisoindolin-4-yl)amino)ethoxy)ethoxy)propyl)-3,5-dimethyl-1H-pyrazol-4-yl)-1-methyl-1H-indole-2-carboxylic acid (18 mg, 0.02 mmol, 15%) as yellow solid.

LCMS (ESI+): m/z 867.6 [M+H]⁺

¹H NMR (400 MHz, DMSO) δ 11.10 (s, 1H), 7.67 (d, J=8.1 Hz, 1H), 7.56 (t, J=7.8 Hz, 1H), 7.12 (dd, J=15.5, 8.0 Hz, 2H), 7.03 (d, J=7.0 Hz, 1H), 6.98 (d, J=6.9 Hz, 1H), 6.78 (s, 2H), 6.67-6.55 (m, 1H), 5.06 (dd, J=12.9, 5.2 Hz, 1H), 4.08 (t, J=5.6 Hz, 2H), 4.00 (t, J=6.2 Hz, 2H), 3.71-3.64 (m, 4H), 3.53-3.45 (m, 7H), 3.42-3.30 (m, 2H), 3.18 (t, J=7.2 Hz, 2H), 2.88 (ddd, J=18.1, 14.3, 3.7 Hz, 1H), 2.62-2.55 (m, 2H), 2.29 (s, 6H), 2.11-1.97 (m, 8H), 1.96 (s, 3H).

### Example 23: 3-(3-(4-chloro-3,5-dimethylphenoxy)propyl)-7-(1-(3-((2-(2,6-dioxopiperidin-3-yl)-1,3-dioxoisoindolin-4-yl)amino)propyl)-3,5-dimethyl-1H-pyrazol-4-yl)-1-(2-morpholinoethyl)-1H-indole-2-carboxylic acid (228)

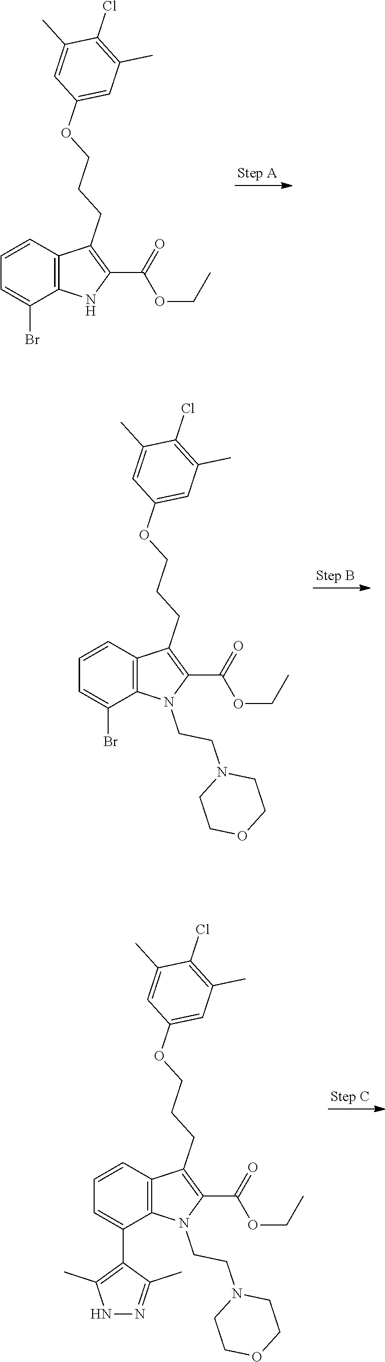

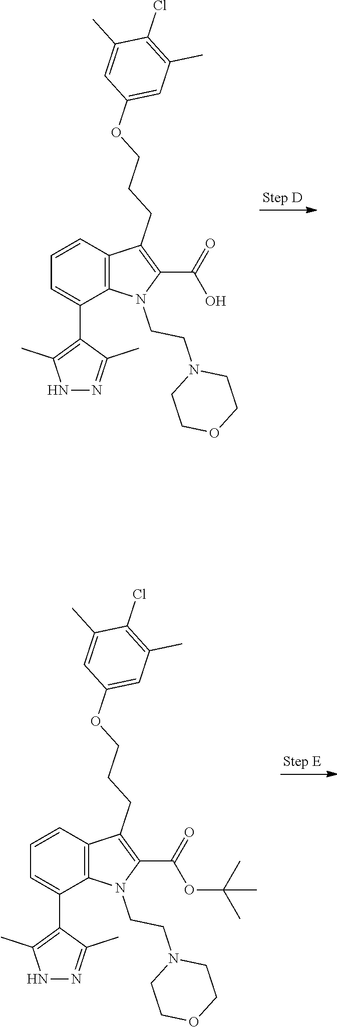 [SMILES: Cc1cc(OCCCc2c(C(=O)O)n(CCN3CCOCC3)c3c(-c4c(C)n[nH]c4C)cccc23)cc(C)c1Cl ; Cc1cc(OCCCc2c(C(=O)OC(C)(C)C)n(CCN3CCOCC3)c3c(-c4c(C)n[nH]c4C)cccc23)cc(C)c1Cl]

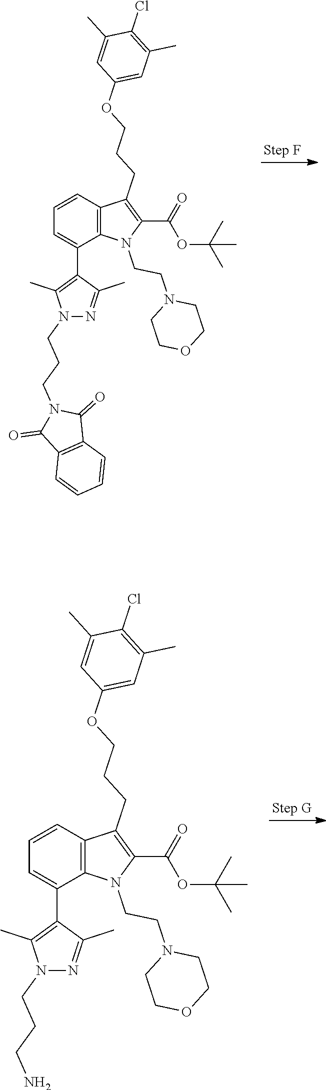

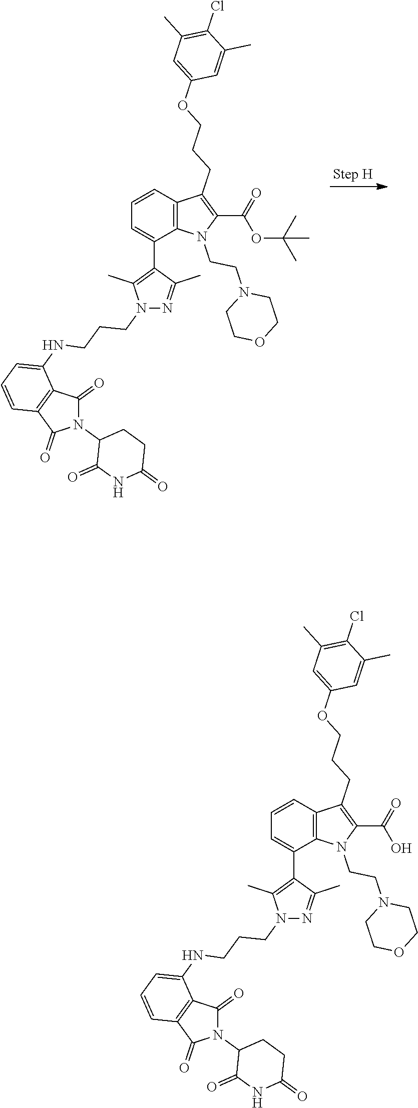 [SMILES: Cc1cc(OCCCc2c(C(=O)OC(C)(C)C)n(CCN3CCOCC3)c3c(-c4c(C)nn(CCCNc5cccc6c5C(=O)N(C5CCC(=O)NC5=O)C6=O)c4C)cccc23)cc(C)c1Cl ; Cc1cc(OCCCc2c(C(=O)O)n(CCN3CCOCC3)c3c(-c4c(C)nn(CCCNc5cccc6c5C(=O)N(C5CCC(=O)NC5=O)C6=O)c4C)cccc23)cc(C)c1Cl]

### Step A

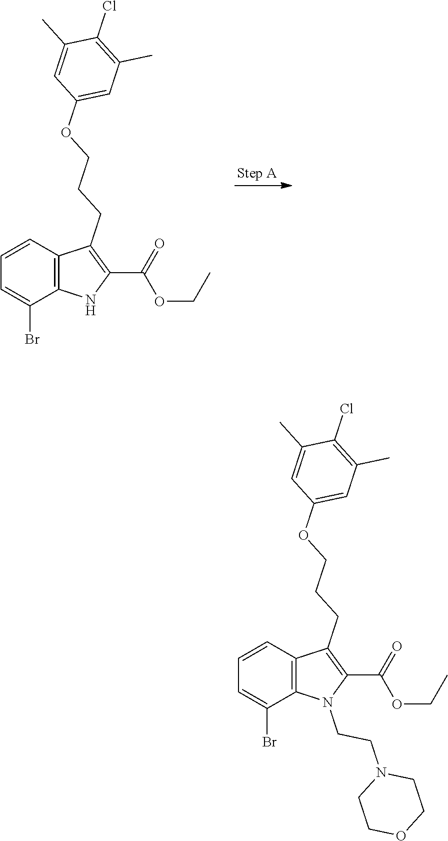 [SMILES: CCOC(=O)c1[nH]c2c(Br)cccc2c1CCCOc1cc(C)c(Cl)c(C)c1 ; CCOC(=O)c1c(CCCOc2cc(C)c(Cl)c(C)c2)c2cccc(Br)c2n1CCN1CCOCC1]

To a well stirred solution of ethyl 7-bromo-3-(3-(4-chloro-3,5-dimethylphenoxy)propyl)-1H-indole-2-carboxylate (5 g, 10.78 mmol) in DMF (50 mL) was added 4-(2-bromoethyl)morpholine (6 g, 30.92 mmol) followed by Cs₂CO₃ (17.5 g, 53.71 mmol) in DMF and the mixture was allowed to stir at 90° C. for 16 h under nitrogen. After complete consumption of the starting material the reaction mixture was diluted with EtOAc, washed successively with water and brine, the organic layer was dried over Na₂SO₄ and evaporated under reduced pressure to get the crude compound, which was then purified by column chromatography (SiO₂, 30% EtOAc in hexane) to get of ethyl 7-bromo-3-(3-(4-chloro-3,5-dimethylphenoxy)propyl)-1-(2-morpholinoethyl)-1H-indole-2-carboxylate (4.8 g, 8.32 mmol, 77%) as off white solid.

LCMS (ESI+): m/z 577.0 [M+H]⁺

### Step B

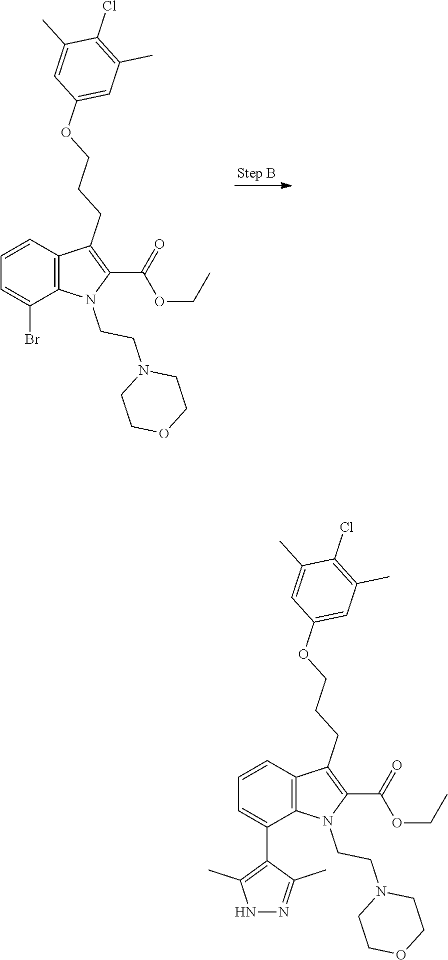 [SMILES: CCOC(=O)c1c(CCCOc2cc(C)c(Cl)c(C)c2)c2cccc(Br)c2n1CCN1CCOCC1 ; CCOC(=O)c1c(CCCOc2cc(C)c(Cl)c(C)c2)c2cccc(-c3c(C)n[nH]c3C)c2n1CCN1CCOCC1]

To a stirred solution of ethyl 7-bromo-3-(3-(4-chloro-3,5-dimethylphenoxy)propyl)-1-(2-morpholinoethyl)-1H-indole-2-carboxylate (4.8 g, 8.32 mmol) in dioxane (150 mL) and water (30 mL) were added 3,5-dimethyl-4-(4,4,5,5-tetramethyl-1,3,2-dioxaborolan-2-yl)-1H-pyrazole (5.54 g, 24.9 mmol) and K₂CO₃ (4.5 g, 32.5 mmol). The mixture was deoxygenated with argon and to it was added Pd(dppf)Cl₂ (1 g, 1.37 mmol) under argon atmosphere. Then the reaction mixture was heated under reflux for 16 h. After complete consumption of the starting material the reaction mixture was filtered through celite pad and the solvents were evaporated under reduced pressure to get the crude material. It was then diluted with EtOAc, washed successively with water and brine, the organic layer was dried over sodium sulphate and evaporated under reduced pressure to get the crude compound, which was then purified by column chromatography (SiO₂, 5% MeOH in DCM) to get ethyl 3-(3-(4-chloro-3,5-dimethylphenoxy)propyl)-7-(3,5-dimethyl-1H-pyrazol-4-yl)-1-(2-morpholinoethyl)-1H-indole-2-carboxylate (2.5 g, 4.21 mmol, 50%) as brown solid.

LCMS (ESI+): m/z 593.8 [M+H]⁺

### Step C

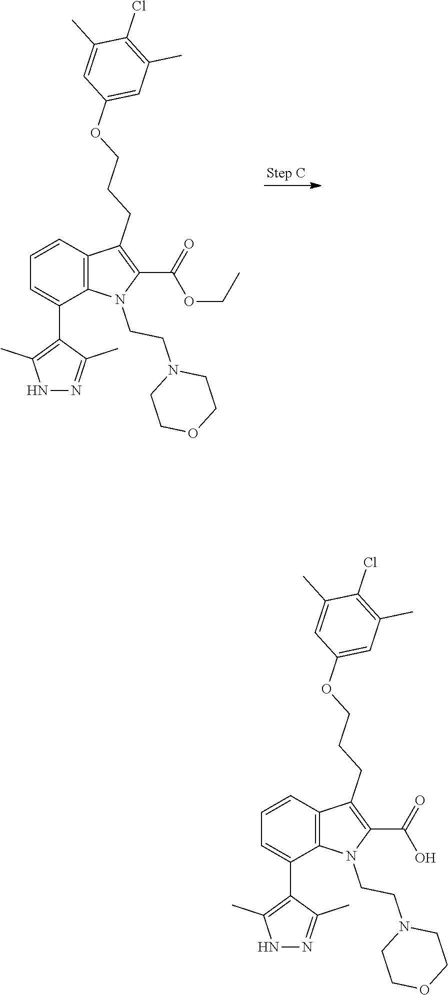 [SMILES: CCOC(=O)c1c(CCCOc2cc(C)c(Cl)c(C)c2)c2cccc(-c3c(C)n[nH]c3C)c2n1CCN1CCOCC1 ; Cc1cc(OCCCc2c(C(=O)O)n(CCN3CCOCC3)c3c(-c4c(C)n[nH]c4C)cccc23)cc(C)c1Cl]

Ethyl 3-(3-(4-chloro-3,5-dimethylphenoxy)propyl)-7-(3,5-dimethyl-1H-pyrazol-4-yl)-1-(2-morpholinoethyl)-1H-indole-2-carboxylate (1.8 g, 3.0 mmol) was dissolved in EtOH (40 mL) and a solution of NaOH (0.43 g, 10.7 mmol) in water (20 mL) was added to it. The mixture was heated under reflux for 3 h. After complete consumption of the starting material the reaction mixture was cooled down to room temperature, solvents were evaporated under reduced pressure to get the crude reaction mixture. It was then diluted with water, washed with EtOAc. Aqueous layer was carefully acidified using 1 (N) HCl to pH=3, extracted with DCM, dried over Na₂SO₄, filtered, and concentrated in vacuo to afford of 3-(3-(4-chloro-3,5-dimethylphenoxy)propyl)-7-(3,5-dimethyl-1H-pyrazol-4-yl)-1-(2-morpholinoethyl)-1H-indole-2-carboxylic acid (1.5 g, 2.65 mmol, 88%) as dark brown gummy solid.

LCMS (ESI+): m/z 565.2 [M+H]⁺

### Step D

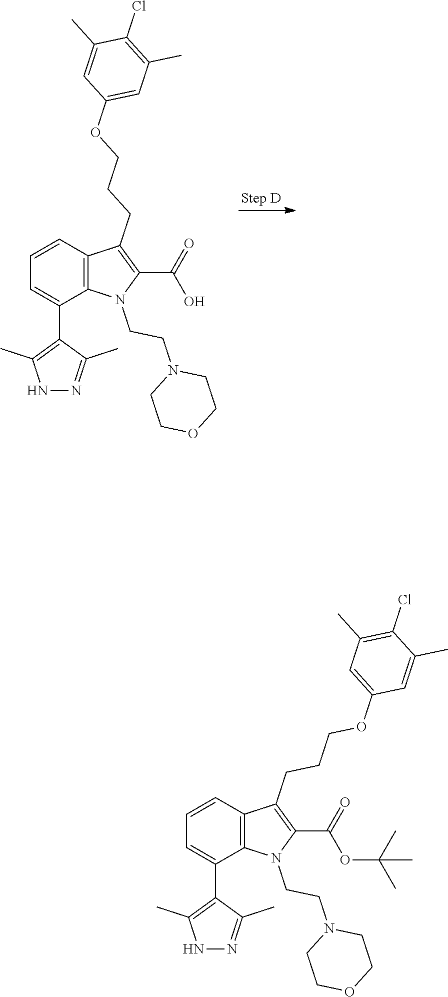

3-(3-(4-chloro-3,5-dimethylphenoxy)propyl)-7-(3,5-dimethyl-1H-pyrazol-4-yl)-1-(2-morpholinoethyl)-1H-indole-2-carboxylic acid (1.5 g, 2.65 mmol) was suspended in toluene (50 mL) and the mixture was heated to reflux under nitrogen. N,N-dimethylformamide di-tert-butyl acetal (0.848 g, 4.2 mmol) was added drop wise to the refluxing mixture. Refluxing was continued for an additional 16 h under nitrogen. After this time another 0.5 mL (2.1 mmol) of N,N-dimethylformamide di-tert-butyl acetal was added to it and the reaction was continued for another 24 h. Reaction mixture was then diluted with EtOAc, washed successively with sodium bicarbonate (saturated), water and brine, organic layer was dried over sodium sulphate and evaporated under reduced pressure to get the crude compound, which was then purified by column chromatography (SiO₂, 6% MeOH in DCM) to get tert-butyl 3-(3-(4-chloro-3,5-dimethylphenoxy)propyl)-7-(3,5-dimethyl-1H-pyrazol-4-yl)-1-(2-morpholinoethyl)-1H-indole-2-carboxylate (800 mg, 1.29 mmol, 49%) as brown solid.

LCMS (ESI+): m/z 621.7 [M+H]⁺

### Step E

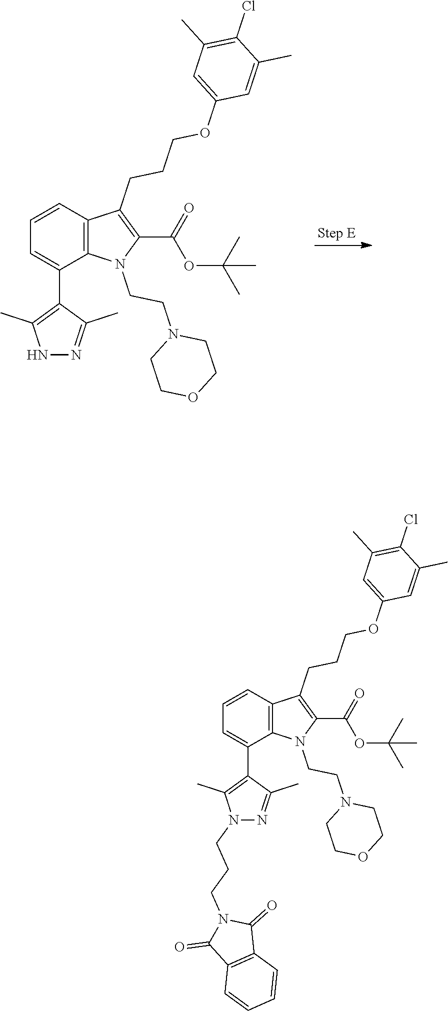

To a stirred solution of tert-butyl 3-(3-(4-chloro-3,5-dimethylphenoxy)propyl)-7-(3,5-dimethyl-1H-pyrazol-4-yl)-1-(2-morpholinoethyl)-1H-indole-2-carboxylate (200 mg, 0.32 mmol) in DMF (1.5 mL) in a pressure tube was added N-(3-bromopropyl)phthalimide (173 mg, 0.64 mmol) followed by cesium carbonate (524 mg, 1.6 mmol) and the reaction mixture was allowed to stir at 100° C. for 16 h. After complete consumption of the starting material the reaction mixture was diluted with EtOAc, washed successively with water and brine, the organic layer was dried over Na₂SO₄ and evaporated under reduced pressure to get the crude compound, which was then purified by column chromatography (SiO₂, 80% EtOAc in DCM) to get tert-butyl 3-(3-(4-chloro-3,5-dimethylphenoxy)propyl)-7-(1-(3-(1,3-dioxoisoindolin-2-yl)propyl)-3,5-dimethyl-1H-pyrazol-4-yl)-1-(2-morpholinoethyl)-1H-indole-2-carboxylate (180 mg, 0.22 mmol, 69%) as gummy solid.

LCMS (ESI+): m/z 808.9 [M+H]⁺

### Step F

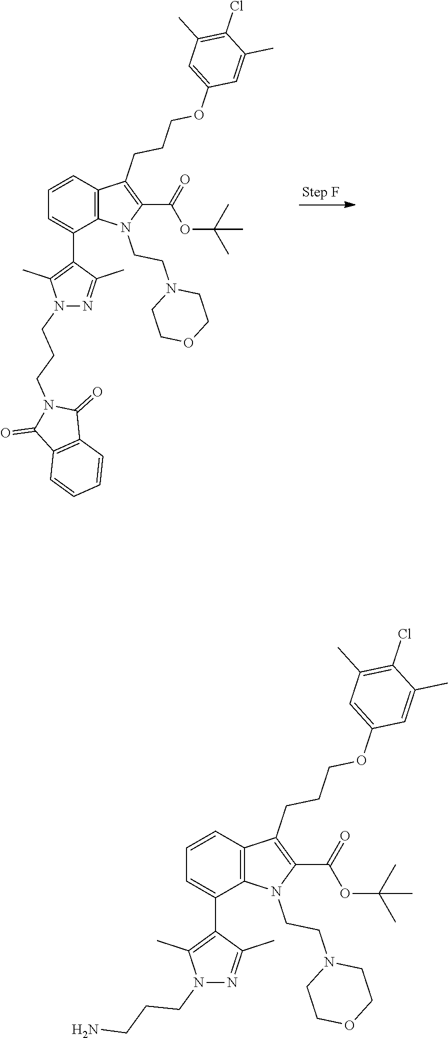 [SMILES: Cc1cc(OCCCc2c(C(=O)OC(C)(C)C)n(CCN3CCOCC3)c3c(-c4c(C)nn(CCCN5C(=O)c6ccccc6C5=O)c4C)cccc23)cc(C)c1Cl ; Cc1cc(OCCCc2c(C(=O)OC(C)(C)C)n(CCN3CCOCC3)c3c(-c4c(C)nn(CCCN)c4C)cccc23)cc(C)c1Cl]

Tert-butyl 3-(3-(4-chloro-3,5-dimethylphenoxy)propyl)-7-(1-(3-(1,3-dioxoisoindolin-2-yl)propyl)-3,5-dimethyl-1H-pyrazol-4-yl)-1-(2-morpholinoethyl)-1H-indole-2-carboxylate (270 mg, 0.33 mmol) and hydrazine hydrate (0.3 mL, 6.7 mmol) in 3 mL of dry tert-butanol was refluxed for 2 h at 140° C. under nitrogen. Then it was allowed to cool down to room temperature, volatiles were removed under reduced pressure, the residue was dissolved in DCM, washed successively with water and brine. The organic layer was dried over MgSO₄, filtered, and evaporated under reduced pressure to get the crude compound which was purified by column chromatography (amine SiO₂, 2% MeOH in DCM) to get tert-butyl 7-(1-(3-aminopropyl)-3,5-dimethyl-1H-pyrazol-4-yl)-3-(3-(4-chloro-3,5-dimethylphenoxy)propyl)-1-(2-morpholinoethyl)-1H-indole-2-carboxylate (160 mg, 0.23 mmol, 70%) as brown liquid.

LCMS (ESI+): m/z 678.8 [M+H]⁺

### Step G

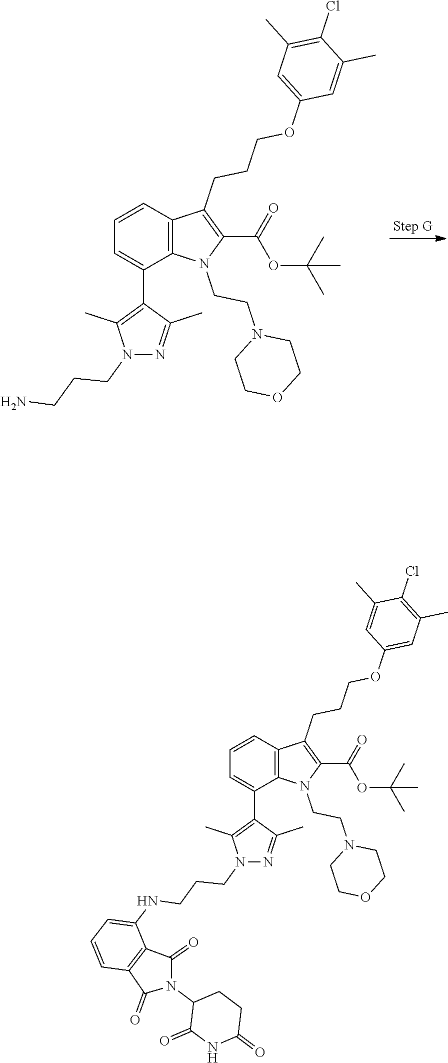 [SMILES: Cc1cc(OCCCc2c(C(=O)OC(C)(C)C)n(CCN3CCOCC3)c3c(-c4c(C)nn(CCCN)c4C)cccc23)cc(C)c1Cl ; Cc1cc(OCCCc2c(C(=O)OC(C)(C)C)n(CCN3CCOCC3)c3c(-c4c(C)nn(CCCNc5cccc6c5C(=O)N(C5CCC(=O)NC5=O)C6=O)c4C)cccc23)cc(C)c1Cl]

To a well stirred solution of tert-butyl 7-(1-(3-aminopropyl)-3,5-dimethyl-1H-pyrazol-4-yl)-3-(3-(4-chloro-3,5-dimethylphenoxy)propyl)-1-(2-morpholinoethyl)-1H-indole-2-carboxylate (170 mg, 0.25 mmol) in DMSO (1 mL) was added 2-(2,6-dioxopiperidin-3-yl)-4-fluoroisoindoline-1,3-dione (104 mg, 0.37 mmol), followed by DIPEA (87 μL, 0.5 mmol) and the reaction mixture was allowed to stir 90° C. for 16 h under nitrogen. After complete consumption of the starting material (monitored by TLC and LCMS) the reaction mixture was diluted with EtOAc, washed successively with water and brine solution, the organic layer was dried over Na₂SO₄ and evaporated under reduced pressure to get the crude compound, which was then purified by column chromatography (amine SiO₂, 80% EtOAc in DCM) to get tert-butyl 3-(3-(4-chloro-3,5-dimethylphenoxy)propyl)-7-(1-(3-((2-(2,6-dioxopiperidin-3-yl)-1,3-dioxoisoindolin-4-yl)amino)propyl)-3,5-dimethyl-1H-pyrazol-4-yl)-1-(2-morpholinoethyl)-1H-indole-2-carboxylate (180 mg, 0.19 mmol, 76%) as yellow solid.

LCMS (ESI+): m/z 935.1 [M+H]⁺

### Step H

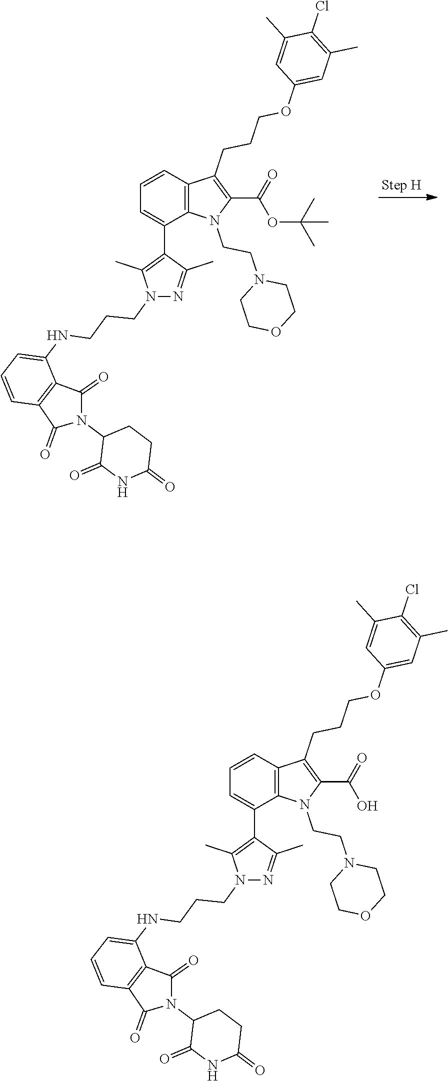 [SMILES: Cc1cc(OCCCc2c(C(=O)OC(C)(C)C)n(CCN3CCOCC3)c3c(-c4c(C)nn(CCCNc5cccc6c5C(=O)N(C5CCC(=O)NC5=O)C6=O)c4C)cccc23)cc(C)c1Cl ; Cc1cc(OCCCc2c(C(=O)O)n(CCN3CCOCC3)c3c(-c4c(C)nn(CCCNc5cccc6c5C(=O)N(C5CCC(=O)NC5=O)C6=O)c4C)cccc23)cc(C)c1Cl]

To a well stirred solution of tert-butyl 3-(3-(4-chloro-3,5-dimethylphenoxy)propyl)-7-(1-(3-((2-(2,6-dioxopiperidin-3-yl)-1,3-dioxoisoindolin-4-yl)amino)propyl)-3,5-dimethyl-1H-pyrazol-4-yl)-1-(2-morpholinoethyl)-1H-indole-2-carboxylate (180 mg, 0.19 mmol) in dioxane (5 mL) was added 5 mL of 4M HCl in dioxane and the reaction mixture was allowed to stir at room temperature for 28 h. After complete consumption of the starting material the reaction mixture was evaporated under reduced pressure to get the crude compound, which was then purified by preparative HPLC (10 mM ammonium acetate in H₂O and MeCN) to get 3-(3-(4-chloro-3,5-dimethylphenoxy)propyl)-7-(1-(3-((2-(2,6-dioxopiperidin-3-yl)-1,3-dioxoisoindolin-4-yl)amino)propyl)-3,5-dimethyl-1H-pyrazol-4-yl)-1-(2-morpholinoethyl)-1H-indole-2-carboxylic acid (45 mg, 0.05 mmol, 26%) as yellow solid.

LCMS (ESI+): m/z 878.4 [M+H]⁺

¹H NMR (400 MHz, DMSO) δ 11.08 (s, 1H), 7.67-7.57 (m, 2H), 7.14 (d, J=8.6 Hz, 1H), 7.09 (t, J=7.5 Hz, 1H), 7.05 (d, J=7.1 Hz, 1H), 6.92 (d, J=7.0 Hz, 1H), 6.75-6.69 (m, 3H), 5.06 (dd, J=12.9, 5.4 Hz, 1H), 4.39-4.20 (m, 2H), 4.12 (t, J=6.8 Hz, 2H), 3.98 (t, J=6.4 Hz, 2H), 3.45-3.37 (m, 3H), 3.14 (t, J=7.5 Hz, 2H), 2.95-2.83 (m, 1H), 2.63-2.53 (m, 2H), 2.25 (s, 6H), 2.14-1.89 (m, 18H).

3 protons in aliphatic area overlaps with water

### Example 24: 3-(3-(4-chloro-3,5-dimethylphenoxy)propyl)-7-(1-(6-((2-(2,6-dioxopiperidin-3-yl)-1,3-dioxoisoindolin-4-yl)amino)hexyl)-3,5-dimethyl-1H-pyrazol-4-yl)-1-(2-morpholinoethyl)-1H-indole-2-carboxylic acid (229)

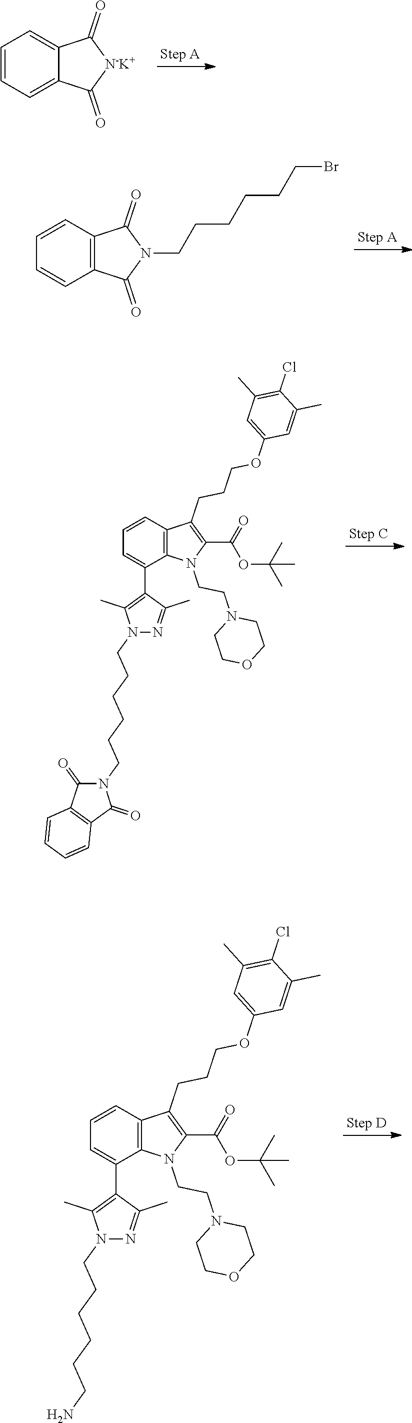

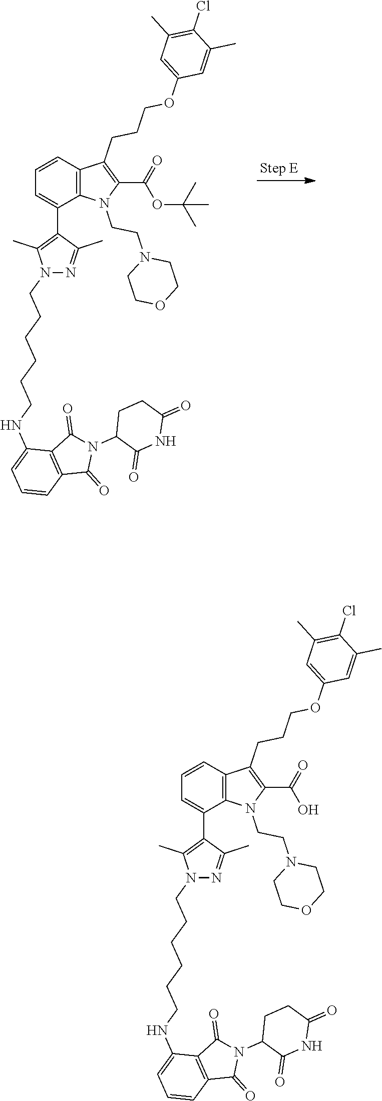 [SMILES: Cc1cc(OCCCc2c(C(=O)OC(C)(C)C)n(CCN3CCOCC3)c3c(-c4c(C)nn(CCCCCCNc5cccc6c5C(=O)N(C5CCC(=O)NC5=O)C6=O)c4C)cccc23)cc(C)c1Cl ; Cc1cc(OCCCc2c(C(=O)O)n(CCN3CCOCC3)c3c(-c4c(C)nn(CCCCCCNc5cccc6c5C(=O)N(C5CCC(=O)NC5=O)C6=O)c4C)cccc23)cc(C)c1Cl]

### Step A

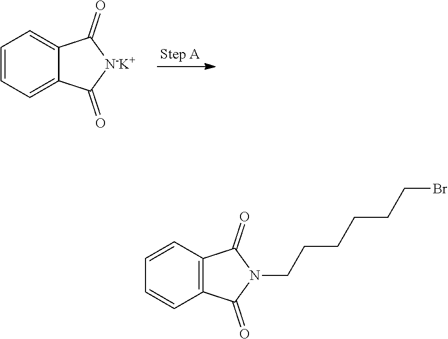

Potassium phthalimide (5 g, 27 mmol) was added in 4 parts to a solution of 1,6-dibromohexane (8.282 mL, 54 mmol) dissolved in boiling acetone (300 mL) and the reaction mixture was allowed to stir at 60° C. for 24 h under nitrogen. After complete consumption of the starting material the solution was cooled to room temperature. The white solid obtained was removed by filtration and the filtrate was concentrated under reduced pressure to get the crude compound, which was then purified by column chromatography (SiO₂, 10% EtOAc in DCM) to get (8 g, 25.8 mmol, 95%) of 2-(6-bromohexyl)isoindoline-1,3-dione as white solid.

LCMS (ESI+): m/z 310.2 [M+H]⁺

### Step B

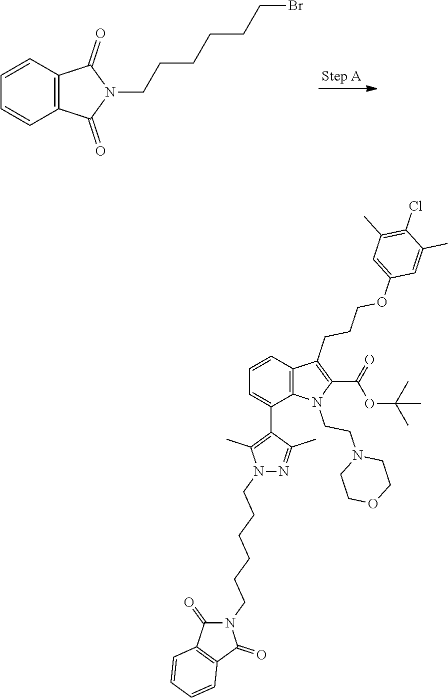 [SMILES: O=C1c2ccccc2C(=O)N1CCCCCCBr ; Cc1cc(OCCCc2c(C(=O)OC(C)(C)C)n(CCN3CCOCC3)c3c(-c4c(C)nn(CCCCCCN5C(=O)c6ccccc6C5=O)c4C)cccc23)cc(C)c1Cl]

To a stirred solution of tert-butyl 3-(3-(4-chloro-3,5-dimethylphenoxy)propyl)-7-(3,5-dimethyl-1H-pyrazol-4-yl)-1-(2-morpholinoethyl)-1H-indole-2-carboxylate (650 mg, 1.05 mmol) in DMF (8 mL) in a pressure tube was added 2-(6-bromohexyl)isoindoline-1,3-dione (650 mg, 2.1 mmol) followed by cesium carbonate (1 g, 3.15 mmol) and the reaction mixture was allowed to stir at 100° C. for 16 h. After complete consumption of the starting material the reaction mixture was diluted with EtOAc, washed successively with water and brine, the organic layer was dried over Na₂SO₄ and evaporated under reduced pressure to get the crude compound, which was then purified by column chromatography (SiO₂, 80% EtOAc in DCM) to get tert-butyl 3-(3-(4-chloro-3,5-dimethylphenoxy)propyl)-7-(1-(6-(1,3-dioxoisoindolin-2-yl)hexyl)-3,5-dimethyl-1H-pyrazol-4-yl)-1-(2-morpholinoethyl)-1H-indole-2-carboxylate (650 mg, crude) as gummy solid.

LCMS (ESI+): m/z 851.0 [M+H]⁺

### Step C

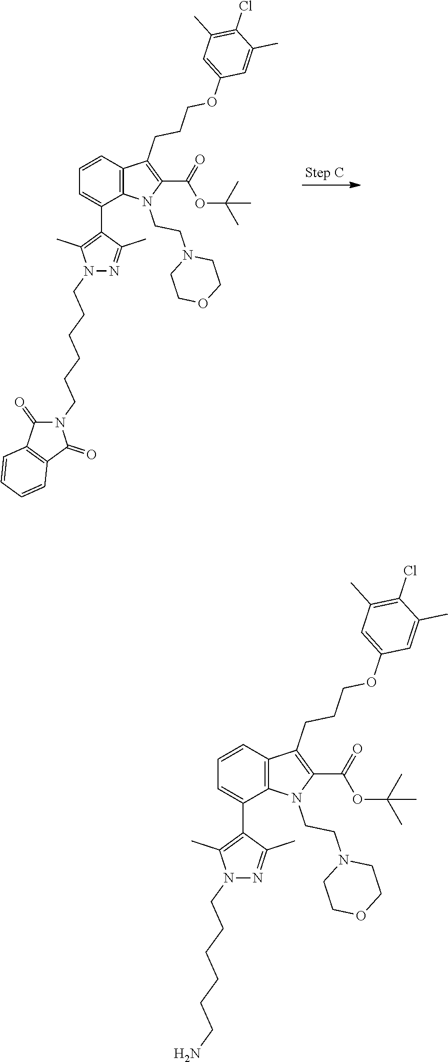

A mixture of tert-butyl 3-(3-(4-chloro-3,5-dimethylphenoxy)propyl)-7-(1-(6-(1,3-dioxoisoindolin-2-yl)hexyl)-3,5-dimethyl-1H-pyrazol-4-yl)-1-(2-morpholinoethyl)-1H-indole-2-carboxylate (500 mg, crude) and hydrazine hydrate (0.3 mL, 6.7 mmol) in 6 mL of dry tert-butanol was refluxed for 2 h at 140° C. under nitrogen. Then it was allowed to cool down to room temperature. Volatiles were evaporated under reduced pressure. The residue was then dissolved in DCM, washed successively with water and brine, the organic layer was dried over MgSO₄, filtered, and evaporated under reduced pressure to get the crude compound which was purified by column chromatography (amine SiO₂, 2% MeOH in DCM) to get tert-butyl 7-(1-(6-aminohexyl)-3,5-dimethyl-1H-pyrazol-4-yl)-3-(3-(4-chloro-3,5-dimethylphenoxy)propyl)-1-(2-morpholinoethyl)-1H-indole-2-carboxylate (300 mg, 0.42 mmol, 70%) as brown liquid.

LCMS (ESI+): m/z 720.9 [M+H]⁺

### Step D

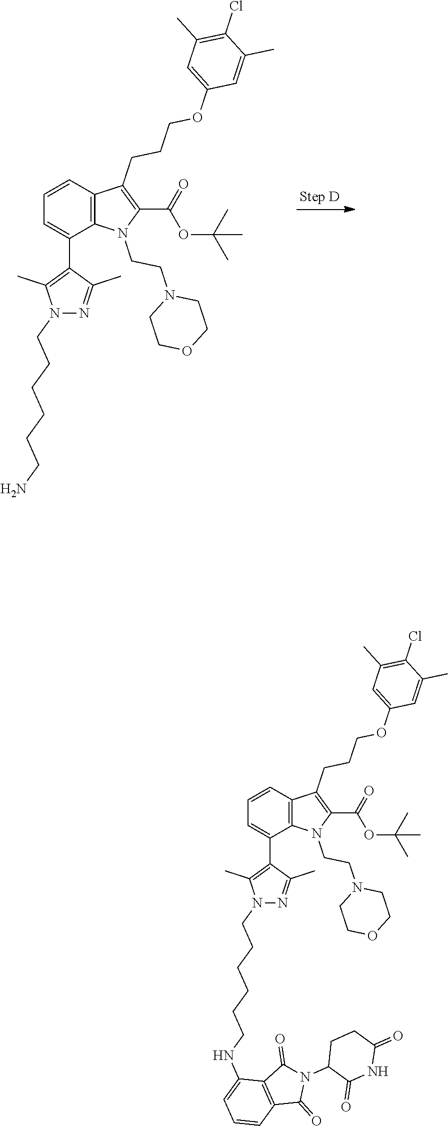

To a well stirred solution of tert-butyl 7-(1-(6-aminohexyl)-3,5-dimethyl-1H-pyrazol-4-yl)-3-(3-(4-chloro-3,5-dimethylphenoxy)propyl)-1-(2-morpholinoethyl)-1H-indole-2-carboxylate (300 mg, 0.38 mmol) in DMSO (3 mL) was added 2-(2,6-dioxopiperidin-3-yl)-4-fluoroisoindoline-1,3-dione (158 mg, 0.57 mmol), followed by DIPEA (133 μL, 0.76 mmol) and the reaction mixture was allowed to stir 90° C. for 16 h under nitrogen. After complete consumption of the starting material the reaction mixture was diluted with EtOAc, washed successively with water and brine solution, the organic layer was dried over sodium sulphate and evaporated under reduced pressure to get the crude compound, which was then purified by column chromatography (amine SiO₂, 80% MeOH in DCM) to get tert-butyl 3-(3-(4-chloro-3,5-dimethylphenoxy)propyl)-7-(1-(6-((2-(2,6-dioxopiperidin-3-yl)-1,3-dioxoisoindolin-4-yl)amino)hexyl)-3,5-dimethyl-1H-pyrazol-4-yl)-1-(2-morpholinoethyl)-1H-indole-2-carboxylate (220 mg, 0.22 mmol, 59%) as yellow solid.

LCMS (ESI+): m/z 977.1 [M+H]⁺

### Step E

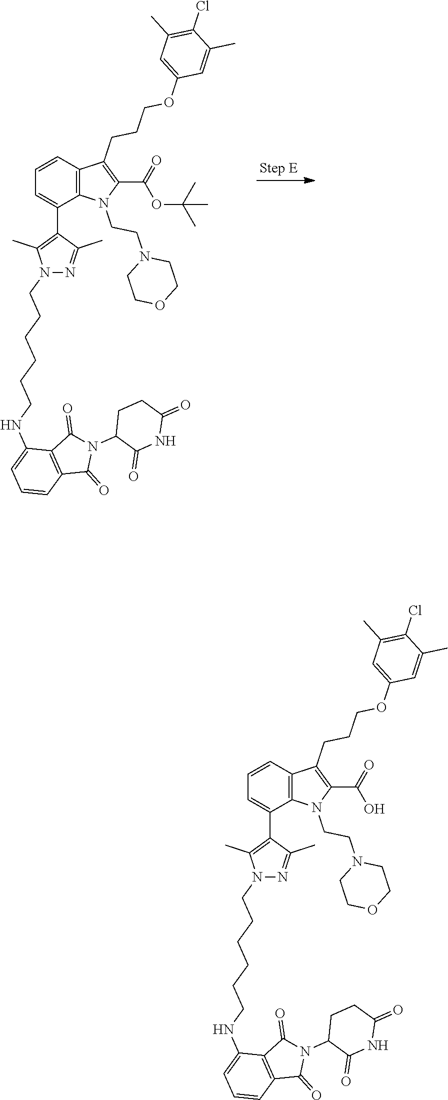 [SMILES: Cc1cc(OCCCc2c(C(=O)OC(C)(C)C)n(CCN3CCOCC3)c3c(-c4c(C)nn(CCCCCCNc5cccc6c5C(=O)N(C5CCC(=O)NC5=O)C6=O)c4C)cccc23)cc(C)c1Cl ; Cc1cc(OCCCc2c(C(=O)O)n(CCN3CCOCC3)c3c(-c4c(C)nn(CCCCCCNc5cccc6c5C(=O)N(C5CCC(=O)NC5=O)C6=O)c4C)cccc23)cc(C)c1Cl]

To a well stirred solution of tert-butyl 3-(3-(4-chloro-3,5-dimethylphenoxy)propyl)-7-(1-(6-((2-(2,6-dioxopiperidin-3-yl)-1,3-dioxoisoindolin-4-yl)amino)hexyl)-3,5-dimethyl-1H-pyrazol-4-yl)-1-(2-morpholinoethyl)-1H-indole-2-carboxylate (220 mg, 0.23 mmol) in dioxane (5 mL) was added 5 mL of 4M HCl in dioxane and the reaction mixture was allowed to stir at room temperature for 28 h. After complete consumption of the starting material the reaction mixture was evaporated under reduced pressure to get the crude compound, which was then purified by preparative HPLC (10 mM ammonium acetate in H₂O and MeCN) to get 3-(3-(4-chloro-3,5-dimethylphenoxy)propyl)-7-(1-(6-((2-(2,6-dioxopiperidin-3-yl)-1,3-dioxoisoindolin-4-yl)amino)hexyl)-3,5-dimethyl-1H-pyrazol-4-yl)-1-(2-morpholinoethyl)-1H-indole-2-carboxylic acid (70 mg, 0.08 mmol, 34%) as yellow solid.

LCMS (ESI+): m/z 920.4 [M+H]⁺

¹H NMR (400 MHz, DMSO) δ 13.29 (s, 1H), 11.08 (s, 1H), 7.64 (d, J=8.0 Hz, 1H), 7.58 (t, J=7.8 Hz, 1H), 7.09 (t, J=7.4 Hz, 2H), 7.02 (d, J=7.0 Hz, 1H), 6.92 (d, J=7.0 Hz, 1H), 6.73 (s, 2H), 6.56 (t, J=6.1 Hz, 1H), 5.04 (dd, J=12.8, 5.3 Hz, 1H), 4.41-4.22 (m, 2H), 3.99 (dt, J=12.3, 6.8 Hz, 4H), 3.37 (t, J=4.6 Hz, 4H), 3.29 (s, 2H), 3.14 (t, J=7.5 Hz, 2H), 2.88 (td, J=17.4, 15.4, 5.2 Hz, 1H), 2.63-2.53 (m, 2H), 2.25 (s, 6H), 2.08-1.89 (m, 15H), 1.76 (t, J=7.2 Hz, 2H), 1.66-1.54 (m, 2H), 1.47-1.28 (m, 4H).

### Example 25: 3-(3-(4-chloro-3,5-dimethylphenoxy)propyl)-7-(1-(3-(2-(2-((2-(2,6-dioxopiperidin-3-yl)-1,3-dioxo-2,3-dihydro-1H-inden-4-yl)amino)ethoxy)ethoxy)propyl)-3,5-dimethyl-1H-pyrazol-4-yl)-1-(2-morpholinoethyl)-1H-indole-2-carboxylic acid (230)

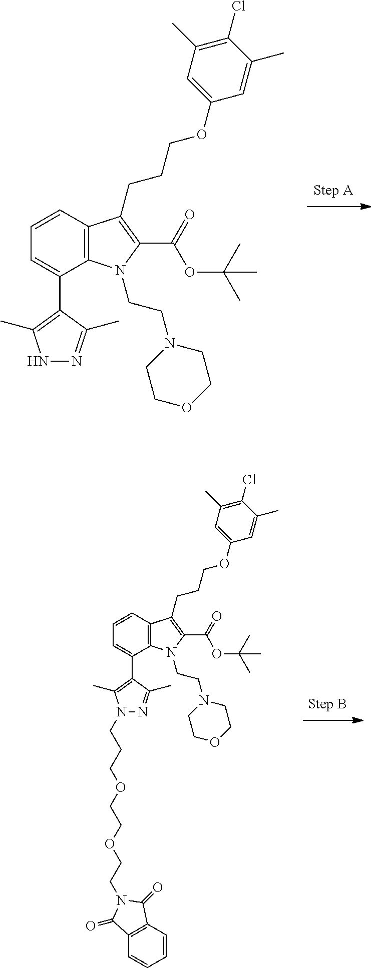

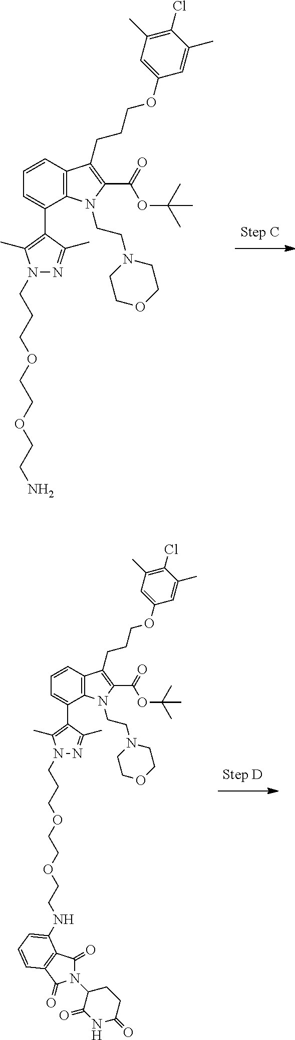

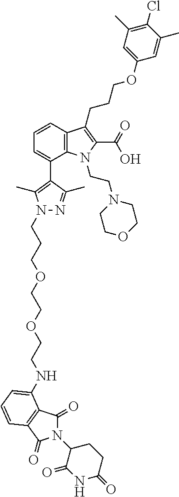 [SMILES: Cc1cc(OCCCc2c(C(=O)O)n(CCN3CCOCC3)c3c(-c4c(C)nn(CCCOCCOCCNc5cccc6c5C(=O)N(C5CCC(=O)NC5=O)C6=O)c4C)cccc23)cc(C)c1Cl]

### Step A

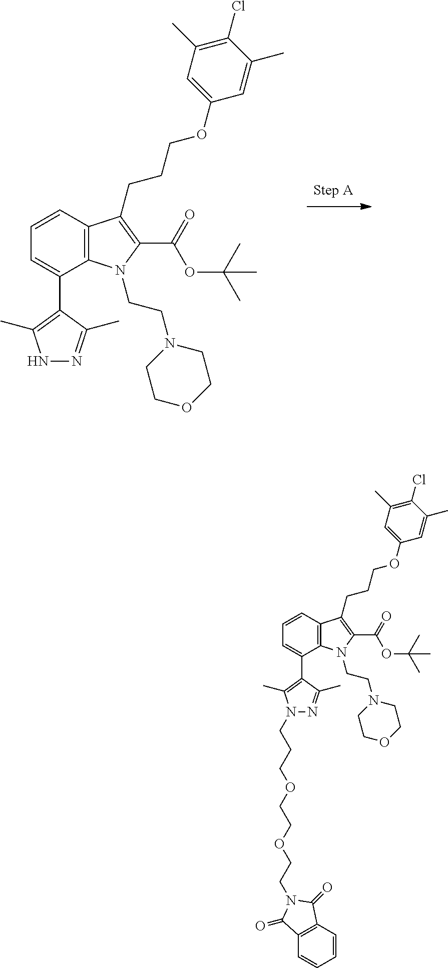

To a stirred solution of tert-butyl 3-(3-(4-chloro-3,5-dimethylphenoxy)propyl)-7-(3,5-dimethyl-1H-pyrazol-4-yl)-1-(2-morpholinoethyl)-1H-indole-2-carboxylate (600 mg, 1.15 mmol) in dry DMF (5 mL) was added NaH (56 mg, 2.3 mmol) portion wise at 0° C. under nitrogen. The reaction mixture was allowed to at the same temperature for 1 h. After that to it was added 2-(2-(2-(3-bromopropoxy)ethoxy)ethyl)isoindoline-1,3-dione (1.2 g, 3.45 mmol) at 0° C. and reaction mixture was allowed to stir at room temperature for 16 h. After complete consumption of the starting material (monitored by TLC and LCMS) the excess NaH was quenched using ice, the volatiles were evaporated under reduced pressure to get the crude compound, which was then purified by column chromatography (SiO₂, 2% MeOH in DCM) to get tert-butyl 3-(3-(4-chloro-3,5-dimethylphenoxy)propyl)-7-(1-(3-(2-(2-(1,3-dioxoisoindolin-2-yl)ethoxy)ethoxy)propyl)-3,5-dimethyl-1H-pyrazol-4-yl)-1-(2-morpholinoethyl)-1H-indole-2-carboxylate (400 mg, 0.44 mmol, 39%) as gummy solid.

### Step B

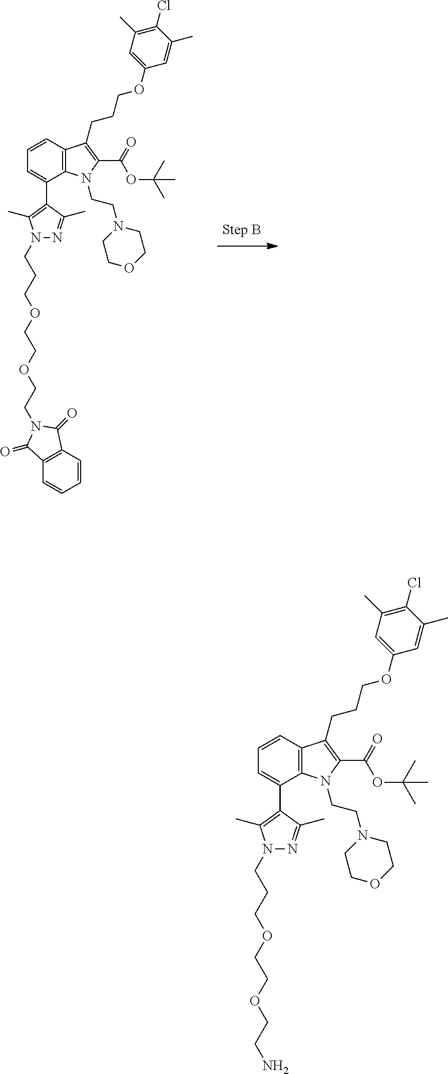

A mixture of tert-butyl 3-(3-(4-chloro-3,5-dimethylphenoxy)propyl)-7-(1-(3-(2-(2-(1,3-dioxoisoindolin-2-yl)ethoxy)ethoxy)propyl)-3,5-dimethyl-1H-pyrazol-4-yl)-1-(2-morpholinoethyl)-1H-indole-2-carboxylate (400 mg, 0.44 mmol) and hydrazine hydrate (0.43 mL, 8.75 mmol) in 10 mL of dry tert-butanol was refluxed for 16 h at 100° C. under nitrogen. Then it was allowed to cool down to room temperature, volatiles were removed under reduced pressure, the residue was dissolved in DCM, washed successively with water and brine. The organic layer was dried over MgSO₄, filtered, and evaporated under reduced pressure to get 350 mg of crude compound (tert-butyl 7-(1-(3-(2-(2-aminoethoxy)ethoxy)propyl)-3,5-dimethyl-1H-pyrazol-4-yl)-3-(3-(4-chloro-3,5-dimethylphenoxy)propyl)-1-(2-morpholinoethyl)-1H-indole-2-carboxylate) which was used for the next step without further purification.

LCMS (ESI+): m/z 767.0 [M+H]⁺

### Step C

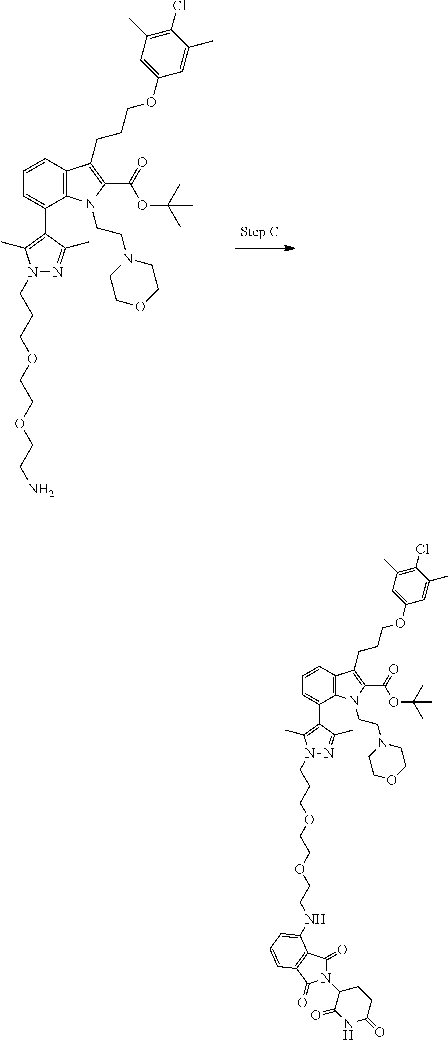

To a well stirred solution of tert-butyl 7-(1-(3-(2-(2-aminoethoxy)ethoxy)propyl)-3,5-dimethyl-1H-pyrazol-4-yl)-3-(3-(4-chloro-3,5-dimethylphenoxy)propyl)-1-(2-morpholinoethyl)-1H-indole-2-carboxylate (350 mg) in DMSO (15 mL) was added 2-(2,6-dioxopiperidin-3-yl)-4-fluoroisoindoline-1,3-dione (190 mg, 0.69 mmol), followed by DIPEA (160 μL, 0.9 mmol) and the reaction mixture was allowed to stir 90° C. for 16 h under nitrogen. After complete consumption of the starting material the reaction mixture was diluted with EtOAc, washed successively with water and brine solution, the organic layer was dried over sodium sulphate and evaporated under reduced pressure to get the crude compound, which was then purified by column chromatography (amine SiO₂, 80% EtOAc in DCM) to get of tert-butyl 3-(3-(4-chloro-3,5-dimethylphenoxy)propyl)-7-(1-(3-(2-(2-((2-(2,6-dioxopiperidin-3-yl)-1,3-dioxo-2,3-dihydro-1H-inden-4-yl)amino)ethoxy)ethoxy)propyl)-3,5-dimethyl-1H-pyrazol-4-yl)-1-(2-morpholinoethyl)-1H-indole-2-carboxylate (150 mg, 0.15 mmol, 32%) as yellow solid.

LCMS (ESI+): m/z 1023.2 [M+H]⁺

### Step D

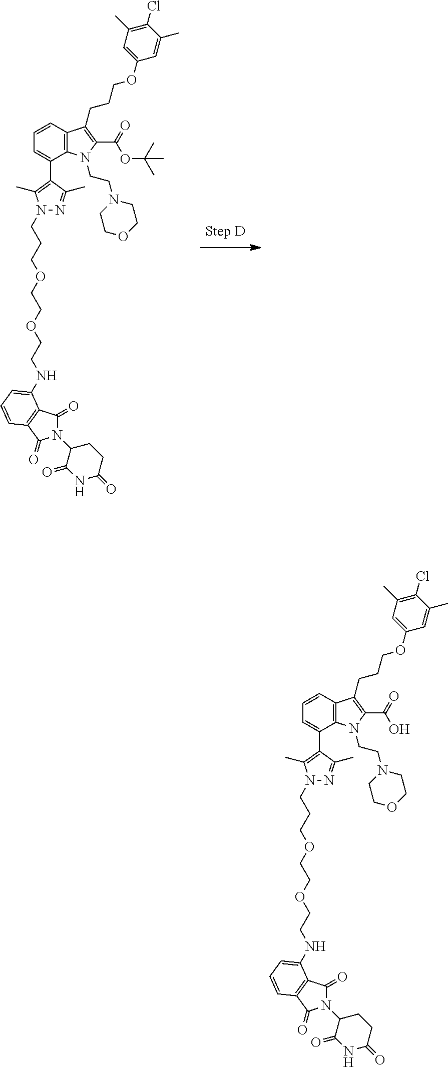 [SMILES: Cc1cc(OCCCc2c(C(=O)OC(C)(C)C)n(CCN3CCOCC3)c3c(-c4c(C)nn(CCCOCCOCCNc5cccc6c5C(=O)N(C5CCC(=O)NC5=O)C6=O)c4C)cccc23)cc(C)c1Cl ; Cc1cc(OCCCc2c(C(=O)O)n(CCN3CCOCC3)c3c(-c4c(C)nn(CCCOCCOCCNc5cccc6c5C(=O)N(C5CCC(=O)NC5=O)C6=O)c4C)cccc23)cc(C)c1Cl]

To a well stirred solution of tert-butyl 3-(3-(4-chloro-3,5-dimethylphenoxy)propyl)-7-(1-(3-(2-(2-((2-(2,6-dioxopiperidin-3-yl)-1,3-dioxo-2,3-dihydro-1H-inden-4-yl)amino)ethoxy)ethoxy)propyl)-3,5-dimethyl-1H-pyrazol-4-yl)-1-(2-morpholinoethyl)-1H-indole-2-carboxylate (150 mg, 0.19 mmol) in dioxane (5 mL) was added 5 mL of 4M HCl in dioxane and the reaction mixture was allowed to stir at room temperature for 28 h. After complete consumption of the starting material the reaction mixture was evaporated under reduced pressure to get the crude compound, which was then divided into two parts. Half of material was purified by reverse phase prep-HPLC (10 mM ammonium acetate in water and acetonitrile) to get of 3-(3-(4-chloro-3,5-dimethylphenoxy)propyl)-7-(1-(3-(2-(2-((2-(2,6-dioxopiperidin-3-yl)-1,3-dioxo-2,3-dihydro-1H-inden-4-yl)amino)ethoxy)ethoxy)propyl)-3,5-dimethyl-1H-pyrazol-4-yl)-1-(2-morpholinoethyl)-1H-indole-2-carboxylic acid as free base (9 mg, 0.009 mmol, 4.7%).

LCMS (ESI+): m/z 966.3 [M+H]⁺

¹H NMR (400 MHz, DMSO) δ 11.10 (s, 1H), 7.73 (d, J=8.0 Hz, 1H), 7.56 (t, J=7.8 Hz, 1H), 7.14 (t, J=7.9 Hz, 2H), 7.05-6.98 (m, 2H), 6.76 (s, 2H), 6.61 (t, J=5.7 Hz, 1H), 5.04 (dd, J=12.8, 5.3 Hz, 1H), 4.57 (s, 2H), 4.14-3.94 (m, 4H), 3.93-3.68 (m, 1H), 3.60 (ddd, J=24.0, 14.7, 5.1 Hz, 8H), 3.45 (dt, J=10.3, 7.9 Hz, 6H), 3.19 (t, J=7.2 Hz, 2H), 2.93-2.81 (m, 1H), 2.57 (dd, J=18.3, 2.6 Hz, 2H), 2.30-2.19 (m, 7H), 2.12-1.85 (m, 12H), 1.76-1.52 (m, 1H), 1.17-1.06 (m, 2H).

### Example 26: 3-[3-(4-chloro-3,5-dimethylphenoxy)propyl]-7-(1-{[(6-{[2-(2,6-dioxopiperidin-3-yl)-1,3-dioxo-2,3-dihydro-1H-isoindol-4-yl]amino}hexyl)carbamoyl]methyl}-1H-1,2,3-triazol-4-yl)-1-[2-(morpholin-4-yl)ethyl]-1H-indole-2-carboxylic acid (231)

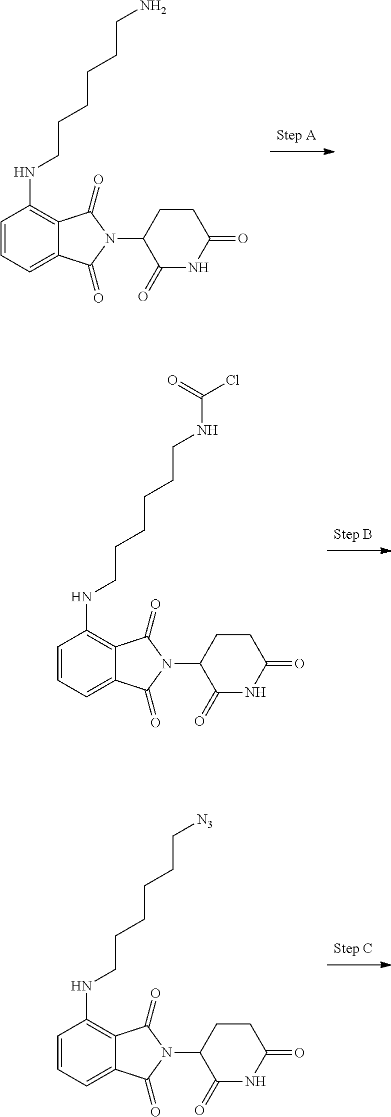 [SMILES: NCCCCCCNc1cccc2c1C(=O)N(C1CCC(=O)NC1=O)C2=O ; O=C(Cl)NCCCCCCNc1cccc2c1C(=O)N(C1CCC(=O)NC1=O)C2=O ; [N-]=[N+]=NCCCCCCNc1cccc2c1C(=O)N(C1CCC(=O)NC1=O)C2=O]

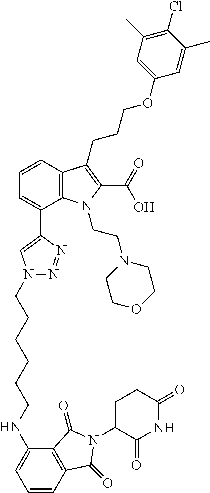 [SMILES: Cc1cc(OCCCc2c(C(=O)O)n(CCN3CCOCC3)c3c(-c4cn(CCCCCCNc5cccc6c5C(=O)N(C5CCC(=O)NC5=O)C6=O)nn4)cccc23)cc(C)c1Cl]

### Step A

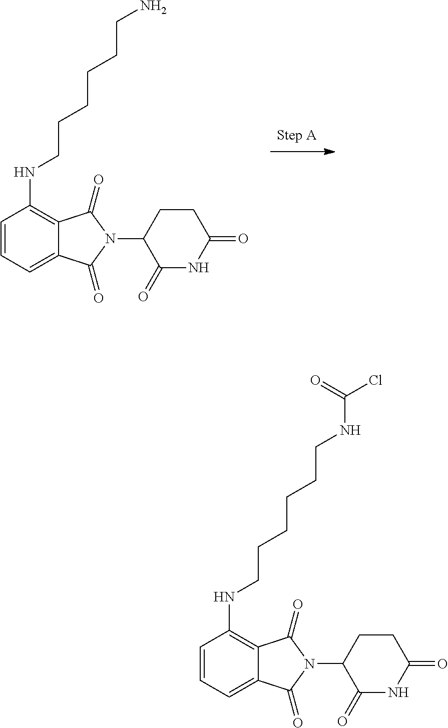 [SMILES: NCCCCCCNc1cccc2c1C(=O)N(C1CCC(=O)NC1=O)C2=O ; O=C(Cl)NCCCCCCNc1cccc2c1C(=O)N(C1CCC(=O)NC1=O)C2=O]

DIPEA (0.213 mL, 1.223 mmol) was added to a stirred solution of 4-[(6-aminohexyl)amino]-2-(2,6-dioxopiperidin-3-yl)-2,3-dihydro-1H-isoindole-1,3-dione hydrochloride (100.0 mg, 0.245 mmol) in DMF (5.0 mL). Subsequently, the mixture was purging by argon for 10 min and chloroacetyl chloride (0.021 mL, 0.269 mmol)_was added. The resulting mixture was stirring at RT for 24 h. After the reaction was completed (monitored by LCMS), the solvent was evaporated and the crude material was purified by flash chromatography (SiO₂, 20% acetone in DCM). The final product 2-chloro-N-(6-{[2-(2,6-dioxopiperidin-3-yl)-1,3-dioxo-2,3-dihydro-1H-isoindol-4-yl]amino}hexyl)acetamide (65.7 mg, 0.146 mmol, 59%) was a yellow oil.

LCMS (ESI+): m/z 449.2 [M+H]⁺

### Step B

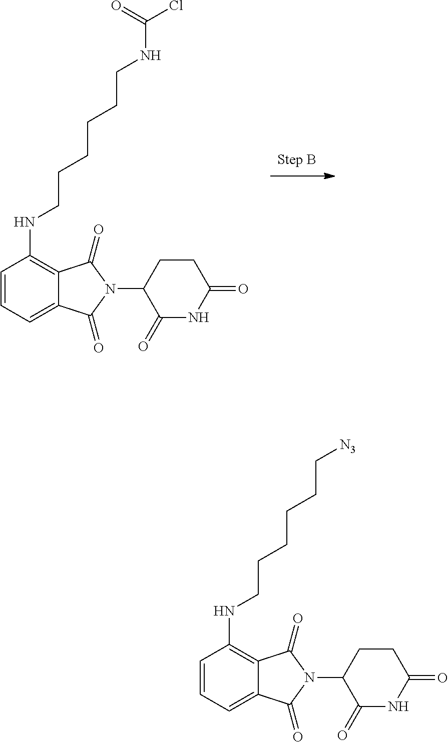 [SMILES: O=C(Cl)NCCCCCCNc1cccc2c1C(=O)N(C1CCC(=O)NC1=O)C2=O ; [N-]=[N+]=NCCCCCCNc1cccc2c1C(=O)N(C1CCC(=O)NC1=O)C2=O]

2-Chloro-N-(6-{[2-(2,6-dioxopiperidin-3-yl)-1,3-dioxo-2,3-dihydro-1H-isoindol-4-yl]amino}hexyl)acetamide (64.0 mg, 0.143 mmol) and NaN₃ (64.9 mg, 0.998 mmol) were dissolved in DMF (2.0 mL). The reaction mixture was stirring in room temperature for 24 h. After this time to the reaction mixture was added H₂O (2 mL) and stirred 15 min. DMF and H₂O was evaporated and the resultant residue was partitioned between DCM and H₂O. The organic layer was further washed with brine, dried over Na₂SO₄, filtered and evaporated. The final product 2-azido-N-(6-{[2-(2,6-dioxopiperidin-3-yl)-1,3-dioxo-2,3-dihydro-1H-isoindol-4-yl]amino}hexyl)acetamide (45.0 mg, 0.099 mmol, 69%) was a yellow solid.

LCMS (ESI+): m/z 455.9 [M+H]⁺

### Step C

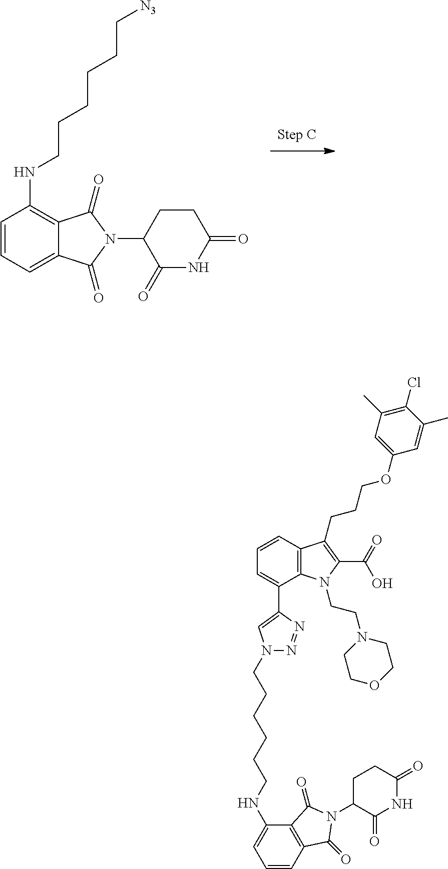 [SMILES: [N-]=[N+]=NCCCCCCNc1cccc2c1C(=O)N(C1CCC(=O)NC1=O)C2=O ; Cc1cc(OCCCc2c(C(=O)O)n(CCN3CCOCC3)c3c(-c4cn(CCCCCCNc5cccc6c5C(=O)N(C5CCC(=O)NC5=O)C6=O)nn4)cccc23)cc(C)c1Cl]

To a solution of 2-azido-N-(6-{[2-(2,6-dioxopiperidin-3-yl)-1,3-dioxo-2,3-dihydro-1H-isoindol-4-yl]amino}hexyl)acetamide (10.1 mg, 0.022 mmol) and 3-[3-(4-chloro-3,5-dimethylphenoxy)propyl]-7-ethynyl-1-[2-(morpholin-4-yl)ethyl]-1H-indole-2-carboxylic acid (10.0 mg, 0.020 mmol) in mixture of H₂O (0.500 mL), EtOH (0.500 mL) and DCM (0.500 mL) was added L-ascorbic acid (3.6 mg, 0.020 mmol) and CuSO₄×5H₂O (2.5 mg, 0.010 mmol). Reaction was stirred at RT and continued for 4 days. Therefore, the solvents were evaporated. Resultant residue was dissolved in DMSO, filtrated and purified by preparative HPLC (H₂O:MeCN+0.1% FA). The final product 3-[3-(4-chloro-3,5-dimethylphenoxy)propyl]-7-(1-{[(6-{[2-(2,6-dioxopiperidin-3-yl)-1,3-dioxo-2,3-dihydro-1H-isoindol-4-yl]amino}hexyl)carbamoyl]methyl}-1H-1,2,3-triazol-4-yl)-1-[2-(morpholin-4-yl)ethyl]-1H-indole-2-carboxylic acid (13.4 mg, 0.014 mmol, 69%) was a yellow solid.

LCMS (ESI+): m/z 950.8 [M+H]⁺

¹H NMR (500 MHz, DMSO) δ 13.44 (s, 1H), 11.08 (s, 1H), 8.35 (t, J=5.4 Hz, 1H), 8.32 (s, 1H), 7.74 (s, 1H), 7.57 (t, J=7.8 Hz, 1H), 7.14-7.07 (m, 3H), 7.01 (d, J=7.0 Hz, 1H), 6.74 (s, 2H), 6.54 (t, J=5.7 Hz, 1H), 5.16 (s, 2H), 5.04 (dd, J=12.7, 5.4 Hz, 1H), 4.40 (t, J=5.6 Hz, 2H), 3.95 (t, J=6.3 Hz, 2H), 3.40-3.37 (m, 4H), 3.18-3.11 (m, 4H), 2.92-2.83 (m, 1H), 2.62-2.53 (m, 2H), 2.26 (s, 6H), 2.07-1.99 (m, 9H), 1.62-1.55 (m, 2H), 1.51-1.44 (m, 2H), 1.36 (d, J=5.0 Hz, 4H), 1.24 (s, 2H).

### Example 27: 7-(1-(3-(2-(2-((2-(2,6-dioxopiperidin-3-yl)-1,3-dioxoisoindolin-4-yl)amino)ethoxy)ethoxy)propyl)-3,5-dimethyl-1H-pyrazol-4-yl)-1-(2-morpholinoethyl)-3-(3-(naphthalen-1-yloxy)propyl)-1H-indole-2-carboxylic acid (232)

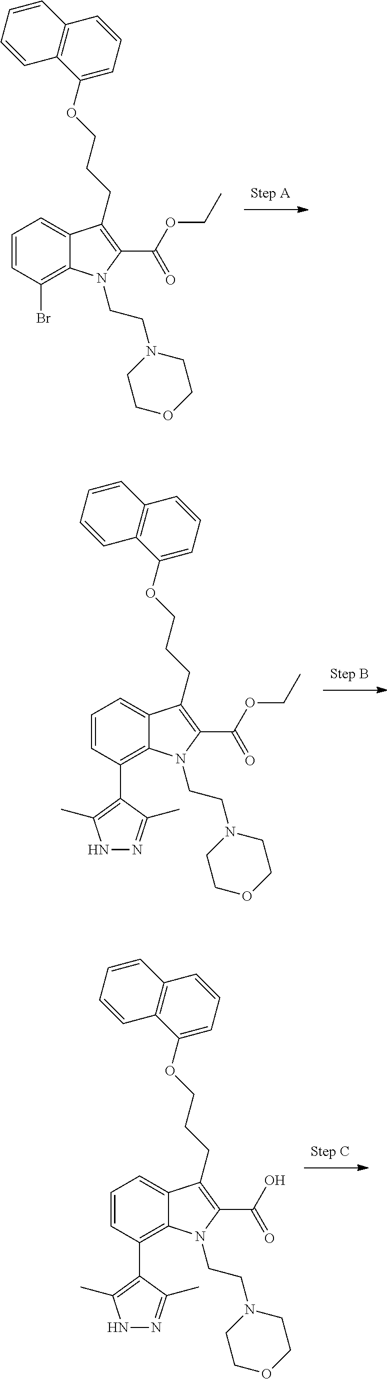

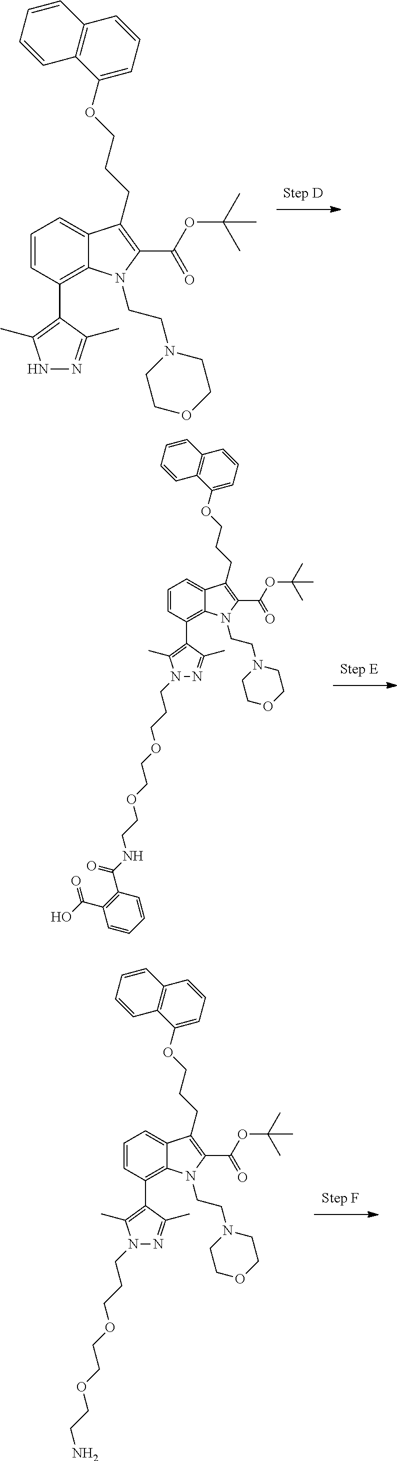

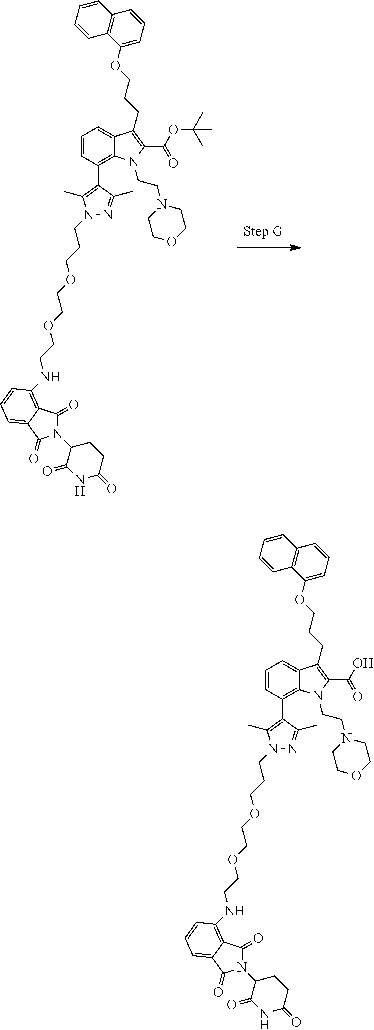 [SMILES: Cc1nn(CCCOCCOCCNc2cccc3c2C(=O)N(C2CCC(=O)NC2=O)C3=O)c(C)c1-c1cccc2c(CCCOc3cccc4ccccc34)c(C(=O)OC(C)(C)C)n(CCN3CCOCC3)c12 ; Cc1nn(CCCOCCOCCNc2cccc3c2C(=O)N(C2CCC(=O)NC2=O)C3=O)c(C)c1-c1cccc2c(CCCOc3cccc4ccccc34)c(C(=O)O)n(CCN3CCOCC3)c12]

### Step A

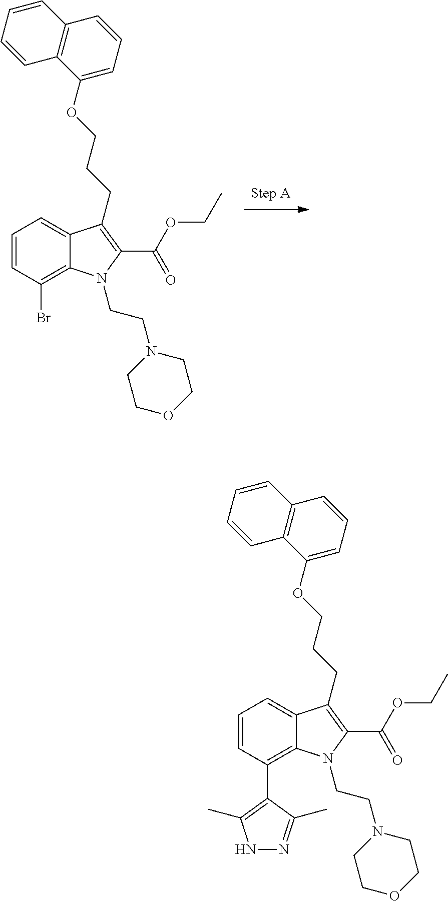 [SMILES: CCOC(=O)c1c(CCCOc2cccc3ccccc23)c2cccc(Br)c2n1CCN1CCOCC1 ; CCOC(=O)c1c(CCCOc2cccc3ccccc23)c2cccc(-c3c(C)n[nH]c3C)c2n1CCN1CCOCC1]

To a stirred solution of ethyl 7-bromo-1-(2-morpholinoethyl)-3-(3-(naphthalen-1-yloxy)propyl)-1H-indole-2-carboxylate (10 g, 17.68 mmol) in dioxane (150 mL) and water (30 mL) were added 3,5-dimethyl-1-(tetrahydro-2H-pyran-2-yl)-4-(4,4,5,5-tetramethyl-1,3,2-dioxaborolan-2-yl)-1H-pyrazole (12 g, 53.05 mmol) and K₂CO₃ (9.7 g, 70.7 mmol). The mixture was deoxygenated with argon and to it was added Pd(dppf)Cl₂ (1.9 g, 2.65 mmol) under argon atmosphere. The reaction mixture was heated under reflux for 16 h. After complete consumption of the starting material the reaction mixture was filtered through celite pad and the solvents were evaporated under reduced pressure get the crude material. It was then diluted with EtOAc and washed successively with water and brine The organic layer was dried over Na₂SO₄ and evaporated under reduced pressure to get the crude compound, which was then purified by column chromatography (SiO₂, 5% MeOH in DCM) to get ethyl 7-(3,5-dimethyl-1H-pyrazol-4-yl)-1-(2-morpholinoethyl)-3-(3-(naphthalen-1-yloxy)propyl)-1H-indole-2-carboxylate (6 g, 10.34 mmol, 58.5%) as brown solid.

LCMS (ESI+): m/z 581.0 [M+H]⁺

### Step B

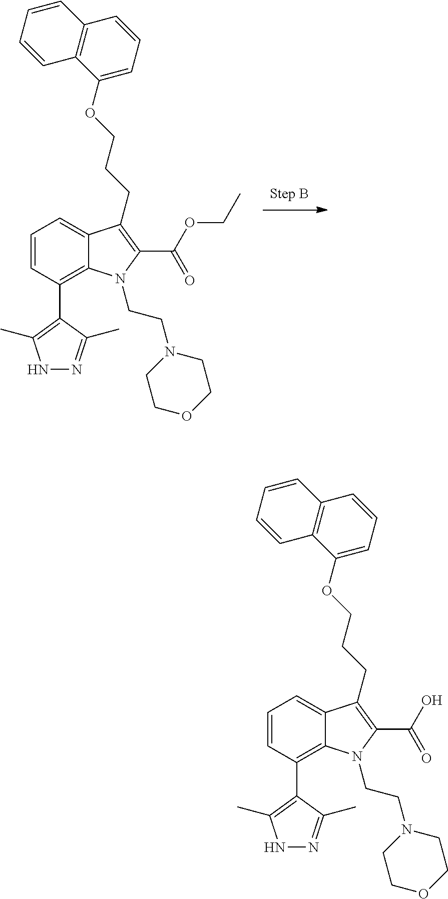 [SMILES: CCOC(=O)c1c(CCCOc2cccc3ccccc23)c2cccc(-c3c(C)n[nH]c3C)c2n1CCN1CCOCC1 ; Cc1n[nH]c(C)c1-c1cccc2c(CCCOc3cccc4ccccc34)c(C(=O)O)n(CCN3CCOCC3)c12]

Ethyl 7-(3,5-dimethyl-1H-pyrazol-4-yl)-1-(2-morpholinoethyl)-3-(3-(naphthalen-1-yloxy)propyl)-1H-indole-2-carboxylate (5 g, 8.4 mmol) was dissolved in EtOH (80 mL) and a solution of NaOH (1.2 g, 29.5 mmol) in water (20 mL) was added to it. The mixture was heated under reflux for 3 h. After complete consumption of the starting material the reaction mixture was cooled down to room temperature and solvents were evaporated under reduced pressure. It was then diluted with water, washed with EtOAc. Aqueous layer was carefully acidified using 1M HCl to pH=3, extracted with DCM (3×50 mL), dried over Na₂SO₄, filtered, and concentrated in vacuo to afford 7-(3,5-dimethyl-1H-pyrazol-4-yl)-1-(2-morpholinoethyl)-3-(3-(naphthalen-1-yloxy)propyl)-1H-indole-2-carboxylic acid (3 g, 5.43 mmol, 64%) as dark brown gummy solid.

LCMS (ESI+): m/z 553.2 [M+H]⁺

### Step C

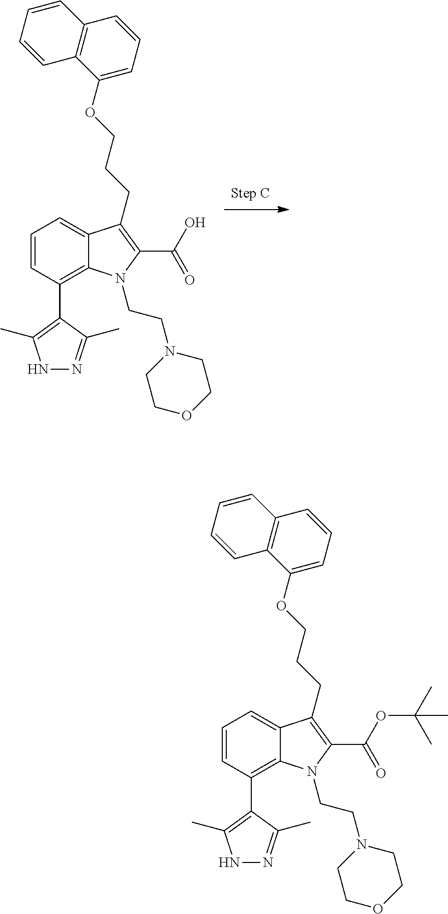 [SMILES: Cc1n[nH]c(C)c1-c1cccc2c(CCCOc3cccc4ccccc34)c(C(=O)O)n(CCN3CCOCC3)c12 ; Cc1n[nH]c(C)c1-c1cccc2c(CCCOc3cccc4ccccc34)c(C(=O)OC(C)(C)C)n(CCN3CCOCC3)c12]

7-(3,5-dimethyl-1H-pyrazol-4-yl)-1-(2-morpholinoethyl)-3-(3-(naphthalen-1-yloxy)propyl)-1H-indole-2-carboxylic acid (2 g, 3.6 mmol) was suspended in toluene (20 mL) and the mixture was heated to reflux under nitrogen. N,N-dimethylformamide di-tert-butyl acetal (3 mL, 14.5 mmol) was added drop-wise to the refluxing mixture. Refluxing was continued for an additional 16 h under nitrogen. After that time another 1.5 mL (7.3 mmol) of N,N-dimethylformamide di-tert-butyl acetal was added to it and the reaction was continued for another 24 h. Reaction mixture was then diluted with EtOAc, washed successively with sodium bicarbonate (saturated aqueous solution), water and brine. Organic layer was dried over sodium sulphate and evaporated under reduced pressure to get the crude compound, which was then purified by column chromatography (SiO₂, 6% MeOH in DCM) to get tert-butyl 7-(3,5-dimethyl-1H-pyrazol-4-yl)-1-(2-morpholinoethyl)-3-(3-(naphthalen-1-yloxy)propyl)-1H-indole-2-carboxylate (1.4 g, 2.3 mmol, 63%) as brown sticky solid.

LCMS (ESI+): m/z 609.0 [M+H]⁺

### Step D

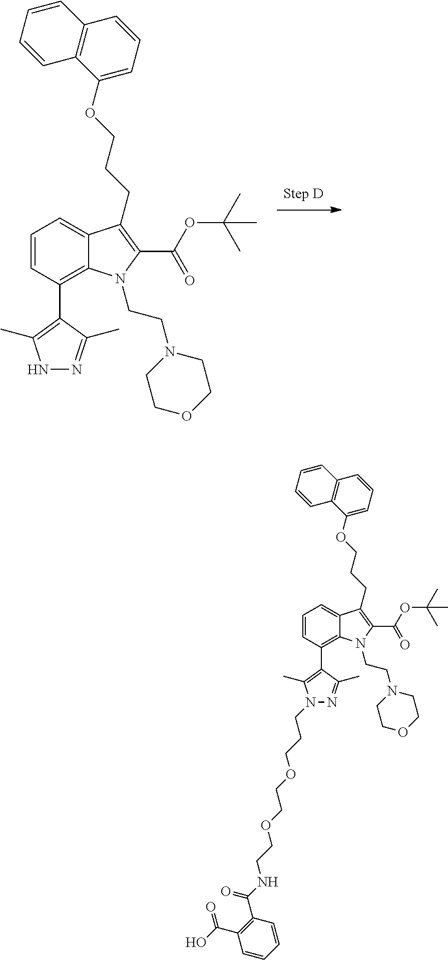 [SMILES: Cc1n[nH]c(C)c1-c1cccc2c(CCCOc3cccc4ccccc34)c(C(=O)OC(C)(C)C)n(CCN3CCOCC3)c12 ; Cc1nn(CCCOCCOCCNC(=O)c2ccccc2C(=O)O)c(C)c1-c1cccc2c(CCCOc3cccc4ccccc34)c(C(=O)OC(C)(C)C)n(CCN3CCOCC3)c12]

To a solution of tert-butyl 7-(3,5-dimethyl-1H-pyrazol-4-yl)-1-(2-morpholinoethyl)-3-(3-(naphthalen-1-yloxy)propyl)-1H-indole-2-carboxylate (500 mg, 0.82 mmol) in DMF (15 mL) at 0° C. was added sodium hydride (197 mg, 8.2 mmol) portion wise under nitrogen and the reaction mixture was allowed to stir at the same temperature for 1 h, after that to the reaction mixture was added 2-(2-(2-(3-bromopropoxy)ethoxy)ethyl)isoindoline-1,3-dione (585 mg, 1.64 mmol) dissolved in DMF (5 mL) at 0° C. and the reaction mixture was allowed to stir at room temperature for 16 h under nitrogen. After complete consumption of the starting material the reaction mixture was again cooled to 0° C. and the excess of NaH was quenched with ammonium chloride (saturated aqueous solution). The volatiles were evaporated under reduced pressure to get the crude 2-((2-(2-(3-(4-(2-(tert-butoxycarbonyl)-1-(2-morpholinoethyl)-3-(3-(naphthalen-1-yloxy)propyl)-1H-indol-7-yl)-3,5-dimethyl-1H-pyrazol-1-yl)propoxy)ethoxy)ethyl)carbamoyl)benzoic acid which was used for the next step without further purification.

LCMS (ESI+): m/z 903.1 [M+H]⁺

### Step E

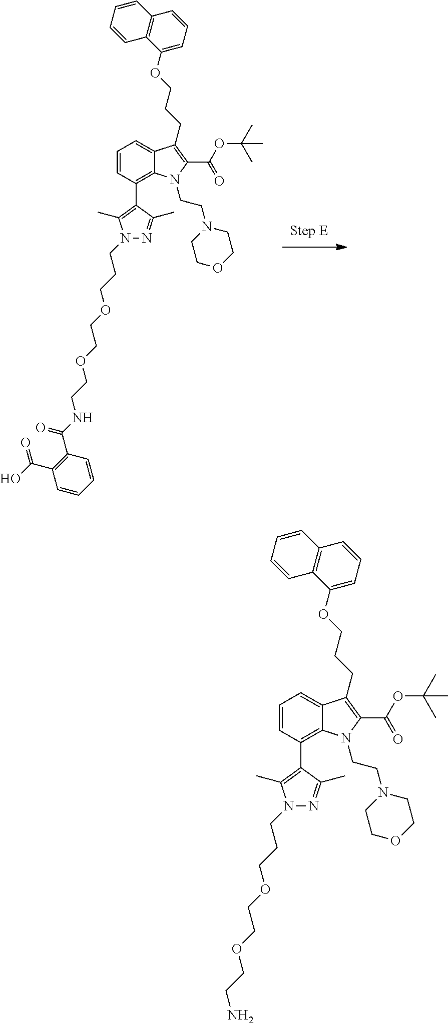 [SMILES: Cc1nn(CCCOCCOCCNC(=O)c2ccccc2C(=O)O)c(C)c1-c1cccc2c(CCCOc3cccc4ccccc34)c(C(=O)OC(C)(C)C)n(CCN3CCOCC3)c12 ; Cc1nn(CCCOCCOCCN)c(C)c1-c1cccc2c(CCCOc3cccc4ccccc34)c(C(=O)OC(C)(C)C)n(CCN3CCOCC3)c12]

A mixture of 2-((2-(2-(3-(4-(2-(tert-butoxycarbonyl)-1-(2-morpholinoethyl)-3-(3-(naphthalen-1-yloxy)propyl)-1H-indol-7-yl)-3,5-dimethyl-1H-pyrazol-1-yl)propoxy)ethoxy)ethyl)carbamoyl)benzoic acid (crude product after step D) and hydrazine hydrate (0.326 mL, 6.65 mmol) in 6 mL of dry tert-butanol was refluxed for 16 h at 140° C. under nitrogen. Then it was allowed to cool down to room temperature and volatiles were evaporated under reduced pressure. The resulting residue was then dissolved in DCM and washed successively with water and brine. The organic layer was dried over magnesium sulfate, filtered and evaporated under reduced pressure to get the crude compound which was purified by column chromatography (amine SiO₂, 2% MeOH in DCM) to get tert-butyl 7-(1-(3-(2-(2-aminoethoxy)ethoxy)propyl)-3,5-dimethyl-1H-pyrazol-4-yl)-1-(2-morpholinoethyl)-3-(3-(naphthalen-1-yloxy)propyl)-1H-indole-2-carboxylate (100 mg, 0.13 mmol, 15% over steps D and E) as brown liquid.

LCMS (ESI+): m/z 754.5 [M+H]⁺

### Step F

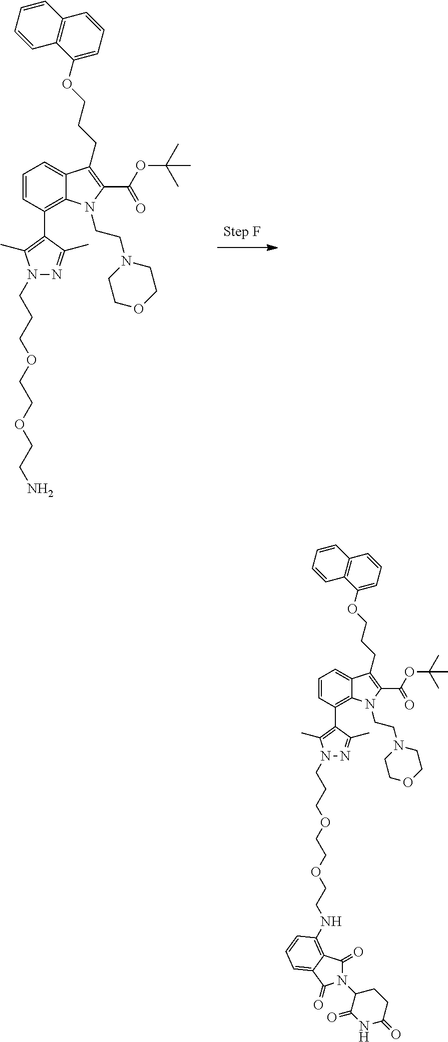 [SMILES: Cc1nn(CCCOCCOCCN)c(C)c1-c1cccc2c(CCCOc3cccc4ccccc34)c(C(=O)OC(C)(C)C)n(CCN3CCOCC3)c12 ; Cc1nn(CCCOCCOCCNc2cccc3c2C(=O)N(C2CCC(=O)NC2=O)C3=O)c(C)c1-c1cccc2c(CCCOc3cccc4ccccc34)c(C(=O)OC(C)(C)C)n(CCN3CCOCC3)c12]

To a solution of tert-butyl 7-(1-(3-(2-(2-aminoethoxy)ethoxy)propyl)-3,5-dimethyl-1H-pyrazol-4-yl)-1-(2-morpholinoethyl)-3-(3-(naphthalen-1-yloxy)propyl)-1H-indole-2-carboxylate (200 mg, 0.27 mmol) in DMSO (1 mL) was added 2-(2,6-dioxopiperidin-3-yl)-4-fluoroisoindoline-1,3-dione (124 mg, 0.45 mmol), followed by DIPEA (104 μL, 0.6 mmol) and the reaction mixture was allowed to stir in 90° C. for 16 h under nitrogen. After complete consumption of the starting material, (monitored by TLC and LCMS), the reaction mixture was diluted with EtOAc, washed successively with water and brine solution, the organic layer was dried over sodium sulphate and evaporated under reduced pressure to get the crude compound, which was then purified by column chromatography (amine SiO₂, 80% EtOAc in DCM) to get tert-butyl 7-(1-(3-(2-(2-((2-(2,6-dioxopiperidin-3-yl)-1,3-dioxoisoindolin-4-yl)amino)ethoxy)ethoxy)propyl)-3,5-dimethyl-1H-pyrazol-4-yl)-1-(2-morpholinoethyl)-3-(3-(naphthalen-1-yloxy)propyl)-1H-indole-2-carboxylate (80 mg, 0.08 mmol, 30%) as yellow solid.

LCMS (ESI+): m/z 1010.8 [M+H]⁺

### Step G

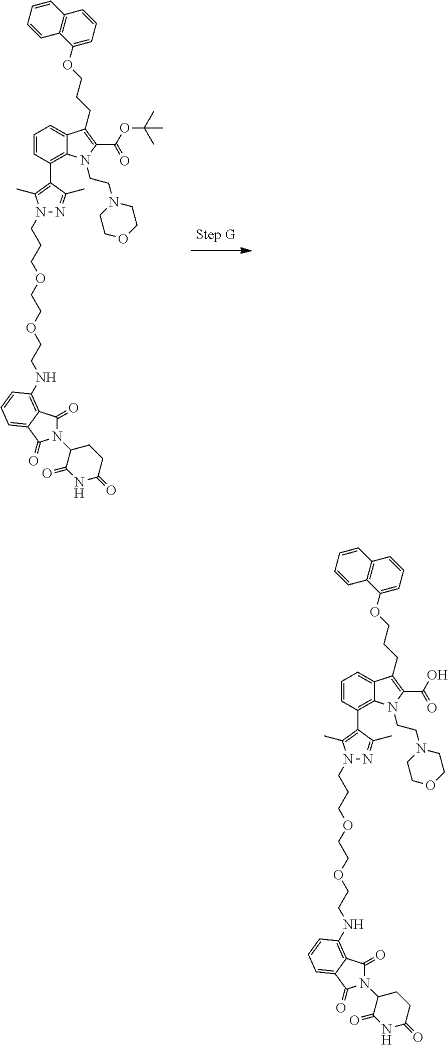 [SMILES: Cc1nn(CCCOCCOCCNc2cccc3c2C(=O)N(C2CCC(=O)NC2=O)C3=O)c(C)c1-c1cccc2c(CCCOc3cccc4ccccc34)c(C(=O)OC(C)(C)C)n(CCN3CCOCC3)c12 ; Cc1nn(CCCOCCOCCNc2cccc3c2C(=O)N(C2CCC(=O)NC2=O)C3=O)c(C)c1-c1cccc2c(CCCOc3cccc4ccccc34)c(C(=O)O)n(CCN3CCOCC3)c12]

To a well stirred solution of tert-butyl 7-(1-(3-(2-(2-((2-(2,6-dioxopiperidin-3-yl)-1,3-dioxoisoindolin-4-yl)amino)ethoxy)ethoxy)propyl)-3,5-dimethyl-1H-pyrazol-4-yl)-1-(2-morpholinoethyl)-3-(3-(naphthalen-1-yloxy)propyl)-1H-indole-2-carboxylate (90 mg, 0.089 mmol) in dioxane (2 mL) was added 4 mL of 4M HCl in dioxane and the reaction mixture was allowed to stir at room temperature for 28 h. After complete consumption of the starting material (monitored by TLC and LCMS) the reaction mixture was evaporated under reduced pressure to get the crude compound, which was then purified by preparative HPLC (H₂O:MeCN+0.1% HCl) to get 7-(1-(3-(2-(2-((2-(2,6-dioxopiperidin-3-yl)-1,3-dioxoisoindolin-4-yl)amino)ethoxy)ethoxy)propyl)-3,5-dimethyl-1H-pyrazol-4-yl)-1-(2-morpholinoethyl)-3-(3-(naphthalen-1-yloxy)propyl)-1H-indole-2-carboxylic acid (17 mg, 0.018 mmol, 20%) as yellow solid.

LCMS (ESI+): m/z 954.9 [M+H]⁺

¹H NMR (400 MHz, DMSO) δ 10.69 (s, 1H), 8.31-8.21 (m, 1H), 7.85 (dd, J=7.0, 2.1 Hz, 1H), 7.74 (d, J=8.0 Hz, 1H), 7.57 (dd, J=8.4, 7.2 Hz, 1H), 7.54-7.48 (m, 2H), 7.48-7.43 (m, 1H), 7.38 (t, J=7.8 Hz, 1H), 7.16-7.10 (m, 2H), 7.04 (d, J=7.1 Hz, 1H), 7.01-6.97 (m, 1H), 6.91 (d, J=7.4 Hz, 1H), 5.00 (dd, J=12.2, 5.4 Hz, 1H), 4.57-4.44 (m, 2H), 4.28 (t, J=6.3 Hz, 2H), 4.16-4.02 (m, 2H), 3.70 (t, J=5.5 Hz, 2H), 3.68-3.61 (m, 6H), 3.61-3.56 (m, 2H), 3.50 (dd, J=11.4, 5.7 Hz, 4H), 3.38-3.32 (m, 3H), 2.91-2.80 (m, 1H), 2.70-2.53 (m, 8H), 2.29-2.24 (m, 2H), 2.08 (s, 3H), 2.07-2.01 (m, 3H), 1.99 (s, 3H).

### Example 28: 3-(3-(4-chloro-3,5-dimethylphenoxy)propyl)-1-(2-(4-((2-(2,6-dioxopiperidin-3-yl)-1,3-dioxoisoindolin-4-yl)glycyl)piperazin-1-yl)ethyl)-7-(1,3,5-trimethyl-1H-pyrazol-4-yl)-1H-indole-2-carboxylic acid (233)

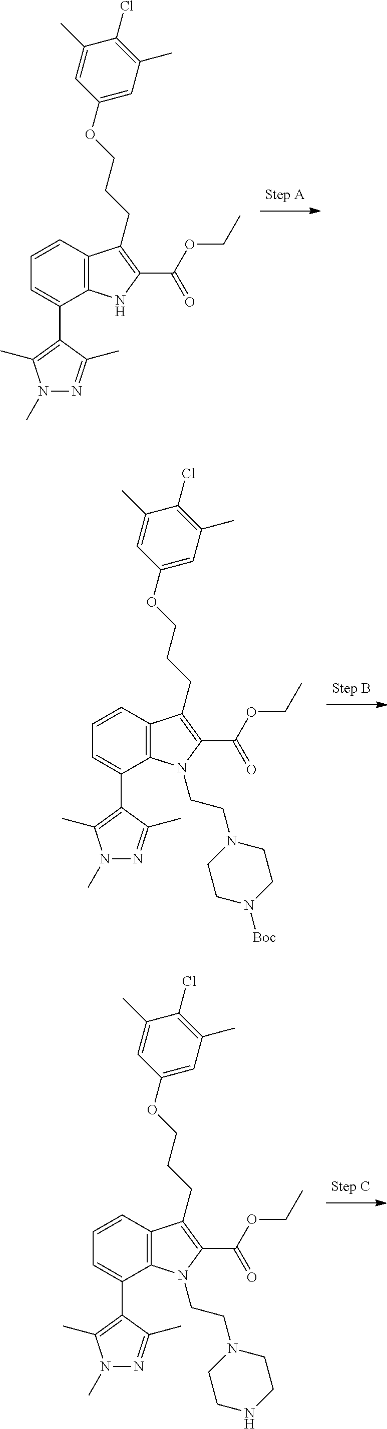

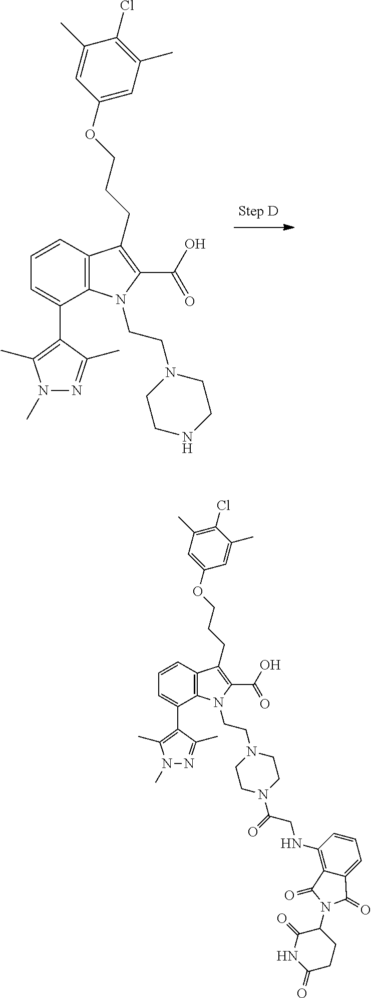 [SMILES: Cc1cc(OCCCc2c(C(=O)O)n(CCN3CCNCC3)c3c(-c4c(C)nn(C)c4C)cccc23)cc(C)c1Cl ; Cc1cc(OCCCc2c(C(=O)O)n(CCN3CCN(C(=O)CNc4cccc5c4C(=O)N(C4CCC(=O)NC4=O)C5=O)CC3)c3c(-c4c(C)nn(C)c4C)cccc23)cc(C)c1Cl]

### Step A

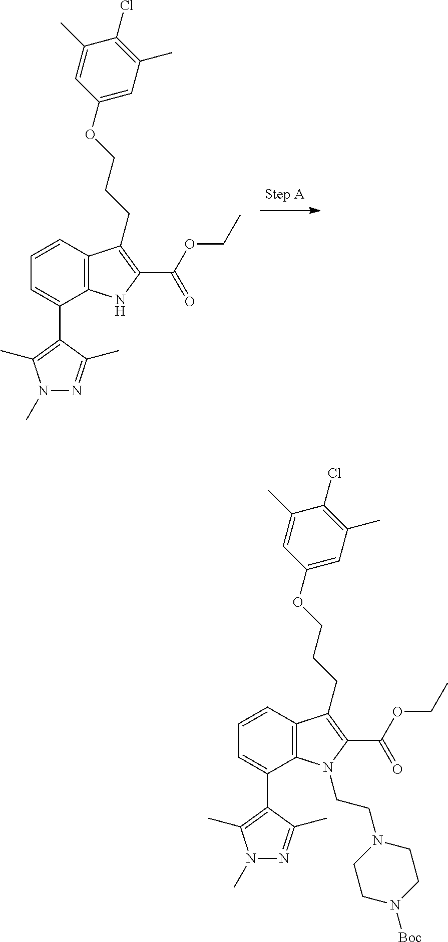 [SMILES: CCOC(=O)c1[nH]c2c(-c3c(C)nn(C)c3C)cccc2c1CCCOc1cc(C)c(Cl)c(C)c1 ; CCOC(=O)c1c(CCCOc2cc(C)c(Cl)c(C)c2)c2cccc(-c3c(C)nn(C)c3C)c2n1CCN1CCN(C(=O)OC(C)(C)C)CC1]

To a mixture of ethyl 3-(3-(4-chloro-3,5-dimethylphenoxy)propyl)-7-(1,3,5-trimethyl-1H-pyrazol-4-yl)-1H-indole-2-carboxylate (300 mg, 0.61 mmol) and K₂CO₃ (252 mg, 1.82 mmol) in DMF (3.00 mL) was added tert-butyl 4-(2-bromoethyl)piperazine-1-carboxylate (214 mg, 0.73 mmol). The reaction vessel was sealed, and heated in 80° C. for 2 days. After that time solvents were removed under reduced pressure, and the crude mixture was separated between EtOAc and brine. The organic layer was collected, concentrated and dried under reduced pressure. The crude product was purified using column chromatography (SiO₂, 10% Acetone in DCM, then 5% MeOH in DCM) to give ethyl 1-(2-(4-(tert-butoxycarbonyl)piperazin-1-yl)ethyl)-3-(3-(4-chloro-3,5-dimethylphenoxy)propyl)-7-(1,3,5-trimethyl-1H-pyrazol-4-yl)-1H-indole-2-carboxylate (185.00 mg, 0.262 mmol, 43%) as light brown semisolid.

LCMS (ESI+): m/z 706.7 [M+H]⁺

### Step B

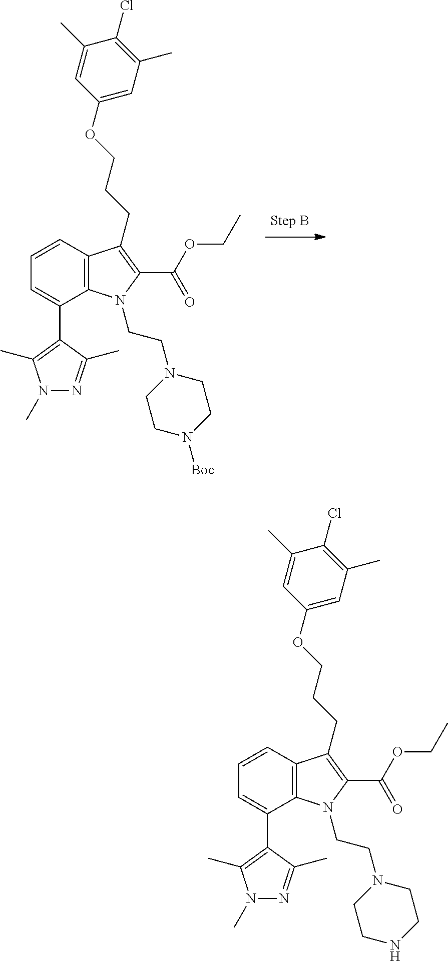 [SMILES: CCOC(=O)c1c(CCCOc2cc(C)c(Cl)c(C)c2)c2cccc(-c3c(C)nn(C)c3C)c2n1CCN1CCN(C(=O)OC(C)(C)C)CC1 ; CCOC(=O)c1c(CCCOc2cc(C)c(Cl)c(C)c2)c2cccc(-c3c(C)nn(C)c3C)c2n1CCN1CCNCC1]

To a solution of ethyl 1-(2-(4-(tert-butoxycarbonyl)piperazin-1-yl)ethyl)-3-(3-(4-chloro-3,5-dimethylphenoxy)propyl)-7-(1,3,5-trimethyl-1H-pyrazol-4-yl)-1H-indole-2-carboxylate (186 mg, 0.26 mmol) in DCM (3.72 mL) was added trifluoroacetic acid (605 μL, 7.90 mmol) and reaction mixture was stirred in RT overnight. Solvents were removed under reduced pressure, NaHCO₃ and DCM were added and crude was extracted with DCM to give ethyl 3-[3-(4-chloro-3,5-dimethylphenoxy)propyl]-1-[2-(piperazin-1-yl)ethyl]-7-(1,3,5-trimethyl-1H-pyrazol-4-yl)-1H-indole-2-carboxylate (140.00 mg, 0.231 mmol, 88%) as light brown semisolid.

LCMS (ESI+): m/z 606.6 [M+H]⁺

### Step C

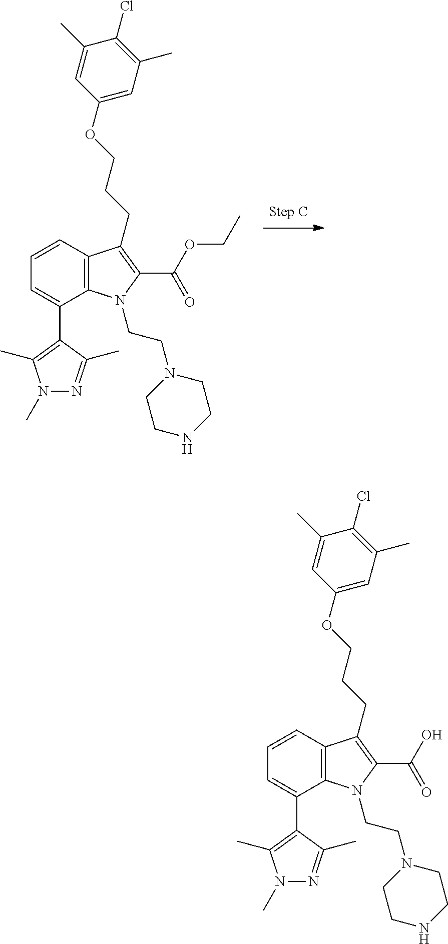 [SMILES: CCOC(=O)c1c(CCCOc2cc(C)c(Cl)c(C)c2)c2cccc(-c3c(C)nn(C)c3C)c2n1CCN1CCNCC1 ; Cc1cc(OCCCc2c(C(=O)O)n(CCN3CCNCC3)c3c(-c4c(C)nn(C)c4C)cccc23)cc(C)c1Cl]

To a solution of ethyl 3-(3-(4-chloro-3,5-dimethylphenoxy)propyl)-1-(2-(piperazin-1-yl)ethyl)-7-(1,3,5-trimethyl-1H-pyrazol-4-yl)-1H-indole-2-carboxylate (0.05 g, 0.08 mmol) in mixture of MeOH:H₂O:THF 1:1:1 (1.20 mL) was added a solution of lithium hydroxide 5% (0.11 mL, 0.23 mmol). The mixture was stirred at 65° C. overnight. The reaction was quenched with a solution of NH₄Cl in H₂O, and was stirred at ambient temperature for 1 hour. The mixture was neutralized with dropwise addition of 0.13 M HCl. Solvent was removed in vacuo and the residue was triturated with CHCl₃. The inorganic salts were filtered off and the filtrate was concentrated in vacuo to afford 3-(3-(4-chloro-3,5-dimethylphenoxy)propyl)-1-(2-(piperazin-1-yl)ethyl)-7-(1,3,5-trimethyl-1H-pyrazol-4-yl)-1H-indole-2-carboxylic acid (48.00 mg, 0.08 mmol, 100%).

LCMS (ESI+): m/z 578.4 [M+H]⁺

### Step D

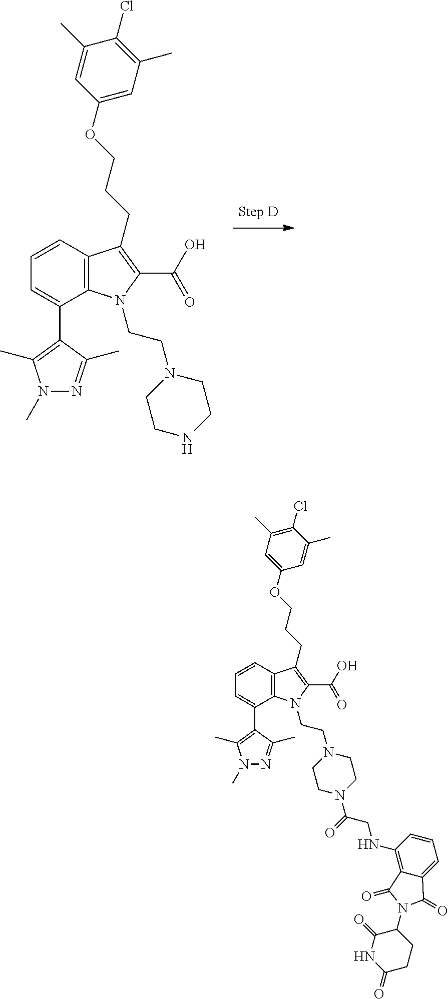 [SMILES: Cc1cc(OCCCc2c(C(=O)O)n(CCN3CCNCC3)c3c(-c4c(C)nn(C)c4C)cccc23)cc(C)c1Cl ; Cc1cc(OCCCc2c(C(=O)O)n(CCN3CCN(C(=O)CNc4cccc5c4C(=O)N(C4CCC(=O)NC4=O)C5=O)CC3)c3c(-c4c(C)nn(C)c4C)cccc23)cc(C)c1Cl]

HATU (33 mg, 0.09 mmol) was mixed with 2-{[2-(2,6-dioxopiperidin-3-yl)-1,3-dioxo-2,3-dihydro-1H-isoindol-4-yl]amino}acetic acid hydrochloride (31 mg, 0.08 mmol) and DIPEA (0.07 mL, 0.42 mmol) in DMF (0.60 mL) for 1 h and then added to the solution of 3-(3-(4-chloro-3,5-dimethylphenoxy)propyl)-1-(2-(piperazin-1-yl)ethyl)-7-(1,3,5-trimethyl-1H-pyrazol-4-yl)-1H-indole-2-carboxylic acid (48 mg, 0.08 mmol) in DMF (0.60 mL) and the mixture was stirred in 70° C. overnight. Crude product was purified using flash chromatography (SiO₂, DCM:MeOH, 0-10%) and repurified using preparative TLC (SiO₂, 10% MeOH in DCM). 3-(3-(4-chloro-3,5-dimethylphenoxy)propyl)-1-(2-(4-((2-(2,6-dioxopiperidin-3-yl)-1,3-dioxoisoindolin-4-yl)glycyl)piperazin-1-yl)ethyl)-7-(1,3,5-trimethyl-1H-pyrazol-4-yl)-1H-indole-2-carboxylic acid (15 mg, 0.017 mmol, 21%) was isolated as yellow solid.

LCMS (ESI+): m/z 891.4 [M+H]⁺

¹H NMR (500 MHz, DMSO) δ 11.09 (s, 1H), 7.65 (d, J=7.8 Hz, 1H), 7.60 (dd, J=8.4, 7.2 Hz, 1H), 7.13-7.02 (m, 4H), 6.92 (d, J=6.7 Hz, 1H), 6.74 (s, 2H), 5.07 (dd, J=12.8, 5.4 Hz, 1H), 4.48-4.39 (m, 1H), 4.31-4.23 (m, 1H), 4.10 (d, J=3.6 Hz, 2H), 4.00 (t, J=6.5 Hz, 2H), 3.76 (s, 3H), 3.42-3.34 (m, 4H), 3.16 (t, J=7.3 Hz, 2H), 2.94-2.85 (m, 1H), 2.62-2.57 (m, 1H), 2.57-2.52 (m, 1H), 2.26 (s, 6H), 2.10-1.99 (m, 12H), 1.94 (s, 3H).

### Example 29: 3-(3-(4-chloro-3,5-dimethylphenoxy)propyl)-1-(2-(4-(2-(2-((2-(2,6-dioxopiperidin-3-yl)-1,3-dioxoisoindolin-4-yl)amino)ethoxy)ethyl)piperazin-1-yl)ethyl)-7-(1,3,5-trimethyl-1H-pyrazol-4-yl)-1H-indole-2-carboxylic acid (234)

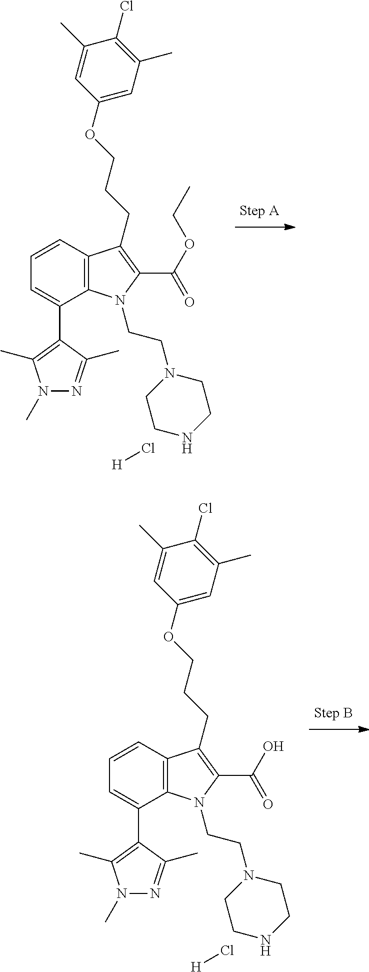

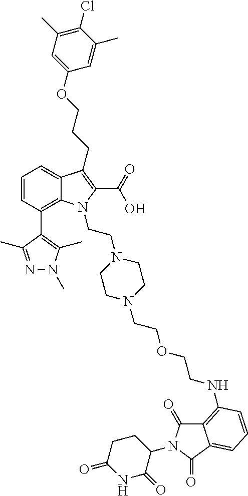 [SMILES: Cc1cc(OCCCc2c(C(=O)O)n(CCN3CCN(CCOCCNc4cccc5c4C(=O)N(C4CCC(=O)NC4=O)C5=O)CC3)c3c(-c4c(C)nn(C)c4C)cccc23)cc(C)c1Cl]

### Step A

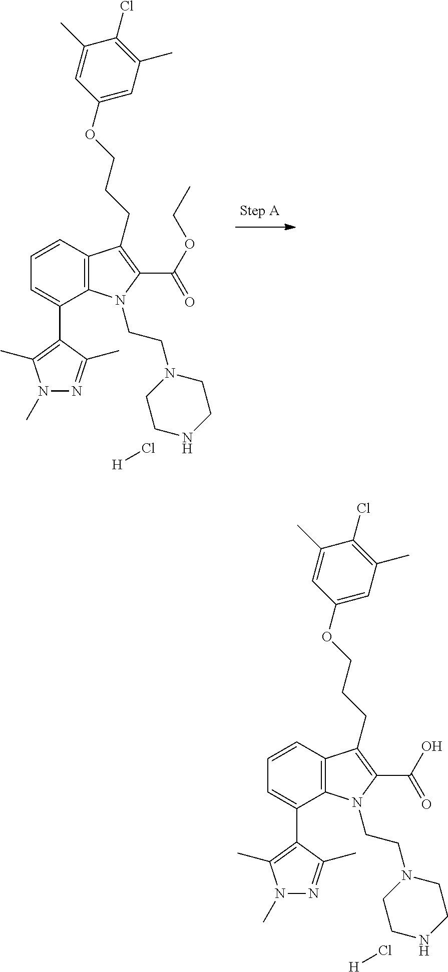

To the solution of ethyl 3-(3-(4-chloro-3,5-dimethylphenoxy)propyl)-1-(2-(piperazin-1-yl)ethyl)-7-(1,3,5-trimethyl-1H-pyrazol-4-yl)-1H-indole-2-carboxylate hydrochloride (6.2 g, 9.6 mmol) in THE (30 mL) and MeOH (100 mL) was added LiOH (3 g) in water (10 mL). The reaction mixture was heated under reflux for 2 h, cooled to RT and concentrated in vacuo. The residue was dissolved in water (200 mL) and washed with EtOAc (100 mL). Water was acidified to neutral pH and extracted with DCM. The organic layer was dried over Na₂SO₄, filtered and concentrated in vacuo. The residue was acidified with dioxane saturated with HCl, the solvent was removed under reduced pressure. The residue was recrystallized from EtOAc to give the 3-[3-(4-chloro-3,5-dimethyl phenoxy)propyl]-1-[2-(piperazin-1-yl)ethyl]-7-(1,3,5-trimethyl-1H-pyrazol-4-yl)-1H-indole-2-carboxylic acid hydrochloride (5.40 g, 8.79 mmol, 91%).

### Step B

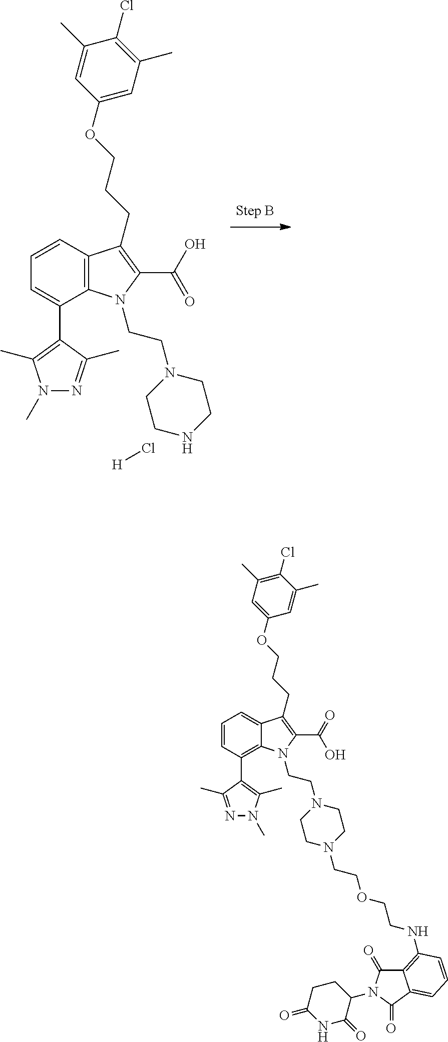

To a solution of 3-[3-(4-chloro-3,5-dimethylphenoxy)propyl]-1-[2-(piperazin-1-yl)ethyl]-7-(1,3,5-trimethyl-1H-pyrazol-4-yl)-1H-indole-2-carboxylic acid hydrochloride (1.00 g, 1.63 mmol) in DMSO (10 mL) were added DIPEA (2.05 mL, 11.73 mmol) and 2-(2-((2-(2,6-dioxopiperidin-3-yl)-1,3-dioxoisoindolin-4-yl)amino)ethoxy)ethyl methanesulfonate (0.86 g, 1.95 mmol). The reaction mixture was stirred at 80° C. (LCMS control). Upon completion, the mixture was poured into water and extracted with CHCl₃. The combined organic layers were dried over anhydrous Na₂SO₄. and concentrated under reduced pressure. Crude product was purified by column chromatography on silica gel to afford 3-(3-(4-chloro-3,5-dimethylphenoxy)propyl)-1-(2-(4-(2-(2-((2-(2,6-dioxopiperidin-3-yl)-1,3-dioxoisoindolin-4-yl)amino)ethoxy)ethyl)piperazin-1-yl)ethyl)-7-(1,3,5-trimethyl-1H-pyrazol-4-yl)-1H-indole-2-carboxylic acid (50 mg, 0.054 mmol, 3%) as yellow solid.

LCMS (ESI+): m/z 921.4 [M+H]⁺

¹H NMR (500 MHz, DMSO) δ 11.09 (s, 1H), 7.63 (d, J=8.0 Hz, 1H), 7.55 (t, J=7.8 Hz, 1H), 7.16-7.06 (m, 2H), 7.02 (d, J=7.0 Hz, 1H), 6.89 (d, 1H), 6.72 (s, 2H), 6.57 (t, J=5.9 Hz, 1H), 5.04 (dd, J=12.8, 5.4 Hz, 1H), 4.42-4.28 (m, 1H), 4.28-4.16 (m, 1H), 3.96 (t, J=6.5 Hz, 2H), 3.71 (s, 3H), 3.55 (t, J=5.4 Hz, 2H), 3.47 (t, J=5.7 Hz, 2H), 3.45-3.42 (m, 2H), 3.12 (t, J=7.4 Hz, 2H), 2.92-2.80 (m, 1H), 2.60-2.54 (m, 2H), 2.42-2.27 (m, 5H), 2.25 (s, 7H), 2.15-1.95 (m, 12H), 1.91 (s, 3H).

### Example 30: 3-(3-(4-chloro-3,5-dimethylphenoxy)propyl)-1-(2-(4-(6-((2-(2,6-dioxopiperidin-3-yl)-1,3-dioxoisoindolin-4-yl)oxy)hexyl)piperazin-1-yl)ethyl)-7-(1,3,5-trimethyl-1H-pyrazol-4-yl)-1H-indole-2-carboxylic acid (235)

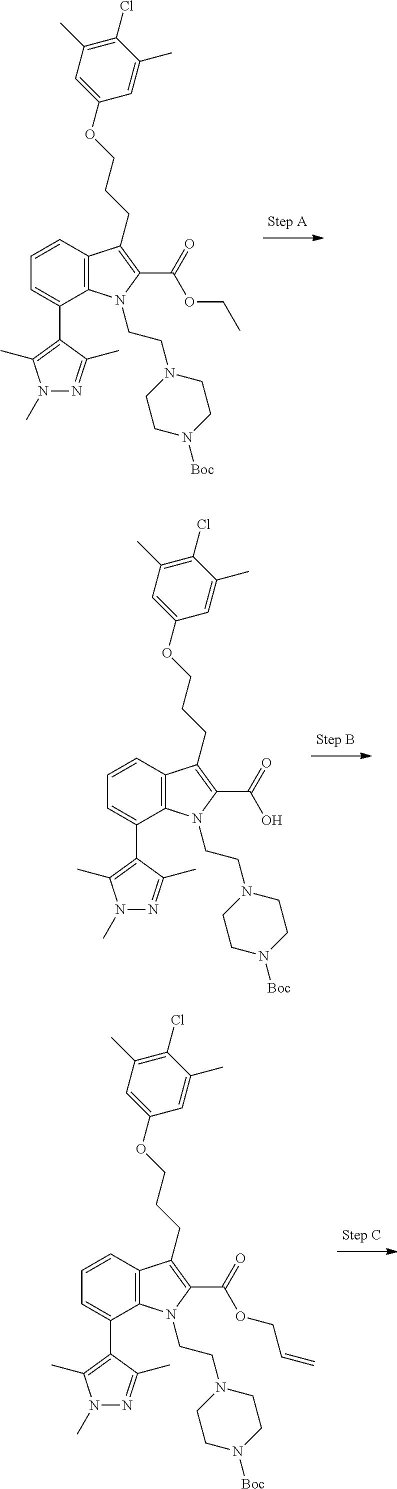

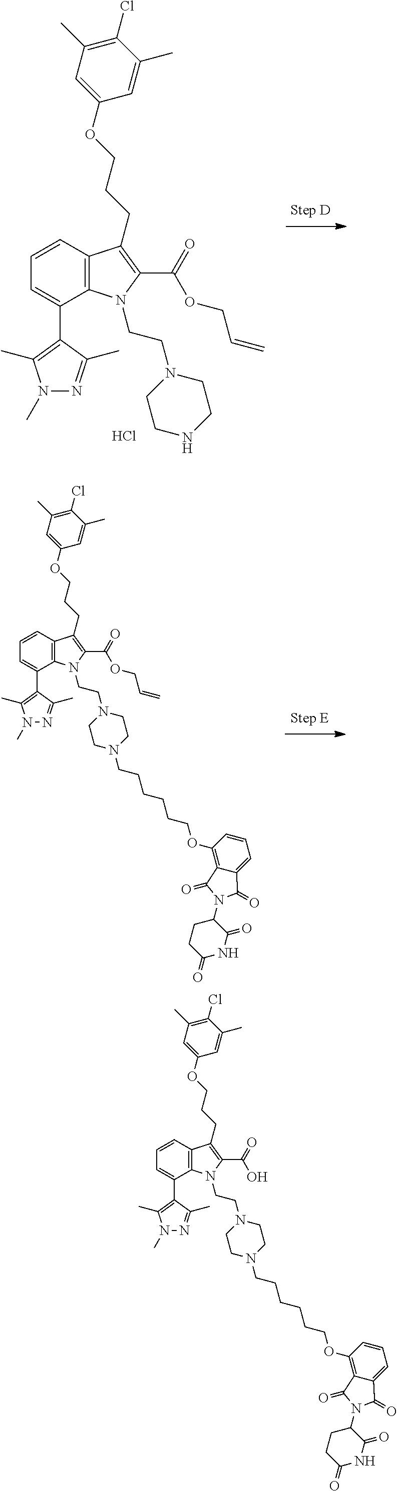

### Step A

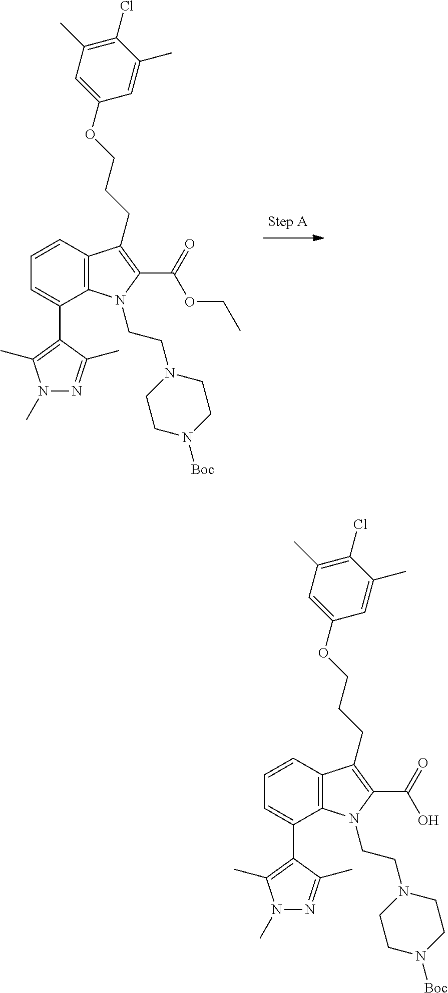 [SMILES: CCOC(=O)c1c(CCCOc2cc(C)c(Cl)c(C)c2)c2cccc(-c3c(C)nn(C)c3C)c2n1CCN1CCN(C(=O)OC(C)(C)C)CC1 ; Cc1cc(OCCCc2c(C(=O)O)n(CCN3CCN(C(=O)OC(C)(C)C)CC3)c3c(-c4c(C)nn(C)c4C)cccc23)cc(C)c1Cl]

To solution of ethyl 1-(2-(4-(tert-butoxycarbonyl)piperazin-1-yl)ethyl)-3-(3-(4-chloro-3,5-dimethylphenoxy)propyl)-7-(1,3,5-trimethyl-1H-pyrazol-4-yl)-1H-indole-2-carboxylate (60 mg, 0.085 mmol) in the mixture of MeOH and H₂O (1:1, 0.42 mL) was added NaOH (27 mg, 0.68 mmol). Mixture was stirred at 50° C. for 3 days. Methanol was removed under reduced pressure and 1M HCl was added. Mixture was extracted 3× with DCM. Organic phases were combined, dried over Na₂SO₄, and concentrated. 1-(2-(4-(tert-butoxycarbonyl)piperazin-1-yl)ethyl)-3-(3-(4-chloro-3,5-dimethylphenoxy)propyl)-7-(1,3,5-trimethyl-1H-pyrazol-4-yl)-1H-indole-2-carboxylic acid (55.00 mg, crude) was used directly in the next step.

LCMS (ESI+): m/z 678.2 [M+H]⁺

### Step B

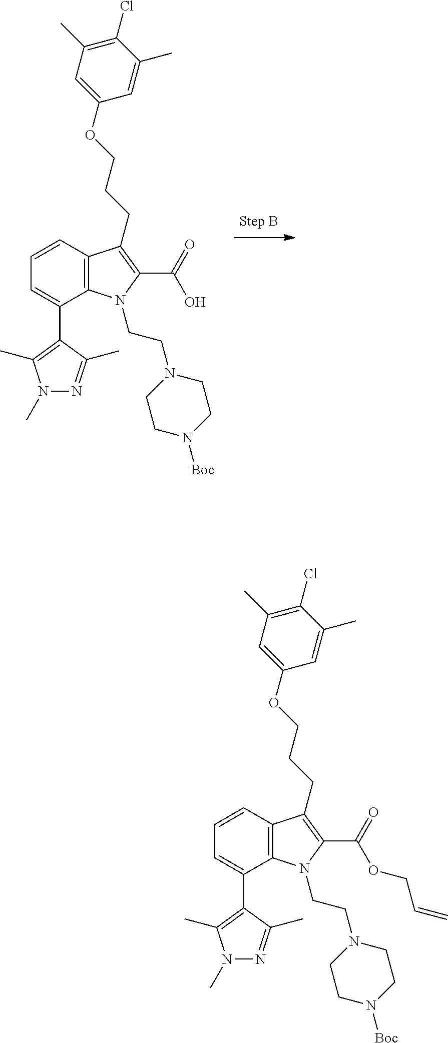 [SMILES: Cc1cc(OCCCc2c(C(=O)O)n(CCN3CCN(C(=O)OC(C)(C)C)CC3)c3c(-c4c(C)nn(C)c4C)cccc23)cc(C)c1Cl ; C=CCOC(=O)c1c(CCCOc2cc(C)c(Cl)c(C)c2)c2cccc(-c3c(C)nn(C)c3C)c2n1CCN1CCN(C(=O)OC(C)(C)C)CC1]

1-(2-(4-(tert-butoxycarbonyl)piperazin-1-yl)ethyl)-3-(3-(4-chloro-3,5-dimethylphenoxy)propyl)-7-(1,3,5-trimethyl-1H-pyrazol-4-yl)-1H-indole-2-carboxylic acid (55 mg, crude) was suspended in dry DMF (0.41 mL) and K₂CO₃ (34 mg, 0.24 mmol) was added. The reaction mixture was stirred at ambient temperature for 5 min, then allyl bromide (13 μL, 0.08 mmol) was added dropwise and the resulting solution was allowed to stir at ambient temperature for 17 h. The reaction mixture was quenched with water and extracted with 3× EtOAc. The combined organic layers were dried over anhydrous Na₂SO₄, filtered and concentrated under reduced to give crude allyl 1-(2-(4-(tert-butoxycarbonyl)piperazin-1-yl)ethyl)-3-(3-(4-chloro-3,5-dimethylphenoxy)propyl)-7-(1,3,5-trimethyl-1H-pyrazol-4-yl)-1H-indole-2-carboxylate (55.00 mg, crude) which was used in next step without further purification.

LCMS (ESI+): m/z 718.3 [M+H]⁺

### Step C

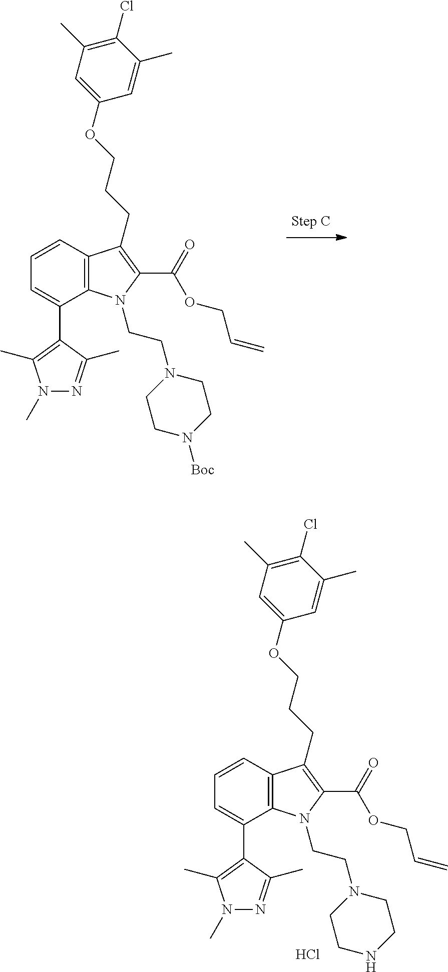 [SMILES: C=CCOC(=O)c1c(CCCOc2cc(C)c(Cl)c(C)c2)c2cccc(-c3c(C)nn(C)c3C)c2n1CCN1CCN(C(=O)OC(C)(C)C)CC1 ; C=CCOC(=O)c1c(CCCOc2cc(C)c(Cl)c(C)c2)c2cccc(-c3c(C)nn(C)c3C)c2n1CCN1CCNCC1 ; Cl]

Allyl 1-(2-(4-(tert-butoxycarbonyl)piperazin-1-yl)ethyl)-3-(3-(4-chloro-3,5-dimethylphenoxy)propyl)-7-(1,3,5-trimethyl-1H-pyrazol-4-yl)-1H-indole-2-carboxylate (55 mg, 0.08 mmol) was dissolved in THE (1.23 mL) and 4M HCl in dioxane (287 μL, 1.15 mmol) was added. Mixture was stirred overnight in RT. Solvents were removed under reduced pressure, and crude co-evaporated three times with Et₂O. Allyl 3-(3-(4-chloro-3,5-dimethylphenoxy)propyl)-1-(2-(piperazin-1-yl)ethyl)-7-(1,3,5-trimethyl-1H-pyrazol-4-yl)-1H-indole-2-carboxylate hydrochloride (50 mg, crude) was used directly in the next step.

### Step D

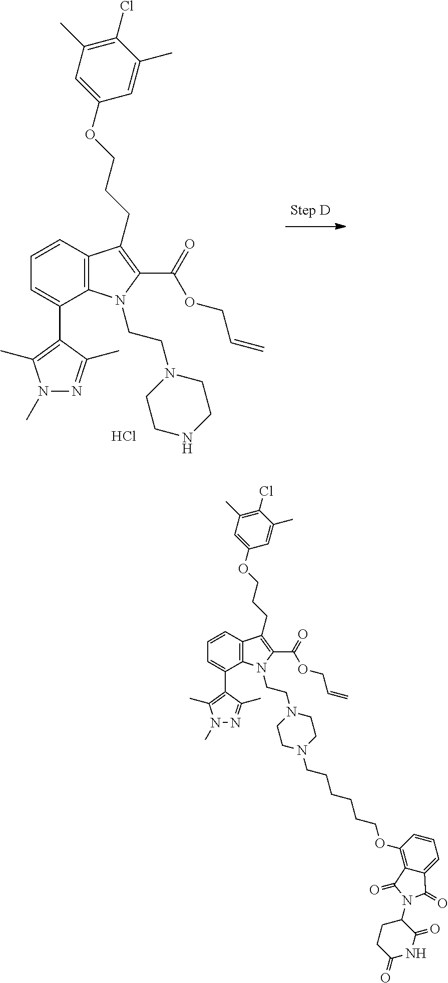 [SMILES: C=CCOC(=O)c1c(CCCOc2cc(C)c(Cl)c(C)c2)c2cccc(-c3c(C)nn(C)c3C)c2n1CCN1CCNCC1 ; Cl ; C=CCOC(=O)c1c(CCCOc2cc(C)c(Cl)c(C)c2)c2cccc(-c3c(C)nn(C)c3C)c2n1CCN1CCN(CCCCCCOc2cccc3c2C(=O)N(C2CCC(=O)NC2=O)C3=O)CC1]

To a solution of allyl 3-(3-(4-chloro-3,5-dimethylphenoxy)propyl)-1-(2-(piperazin-1-yl)ethyl)-7-(1,3,5-trimethyl-1H-pyrazol-4-yl)-1H-indole-2-carboxylate hydrochloride (59 mg, crude) KHCO₃ (36 mg, 0.36 mmol) and KI (15 mg, 0.09 mmol) in DMF (0.64 mL), was added 4-((6-chlorohexyl)oxy)-2-(2,6-dioxopiperidin-3-yl)isoindoline-1,3-dione (57 mg, 0.14 mmol). The reaction mixture was stirred at 60° C. for 2 days. Solvents were removed under reduced pressure, Mixture was suspended in DCM and filtered through Celite. DCM was removed, and solid was washed from Celite using DCM:THF 1:1. Solvents were removed under reduced pressure and allyl 3-(3-(4-chloro-3,5-dimethylphenoxy)propyl)-1-(2-(4-(6-((2-(2,6-dioxopiperidin-3-yl)-1,3-dioxoisoindolin-4-yl)oxy)hexyl)piperazin-1-yl)ethyl)-7-(1,3,5-trimethyl-1H-pyrazol-4-yl)-1H-indole-2-carboxylate (88.00 mg, crude) was used directly in the next step.

LCMS (ESI+): m/z 973.9 [M+H]⁺

### Step E

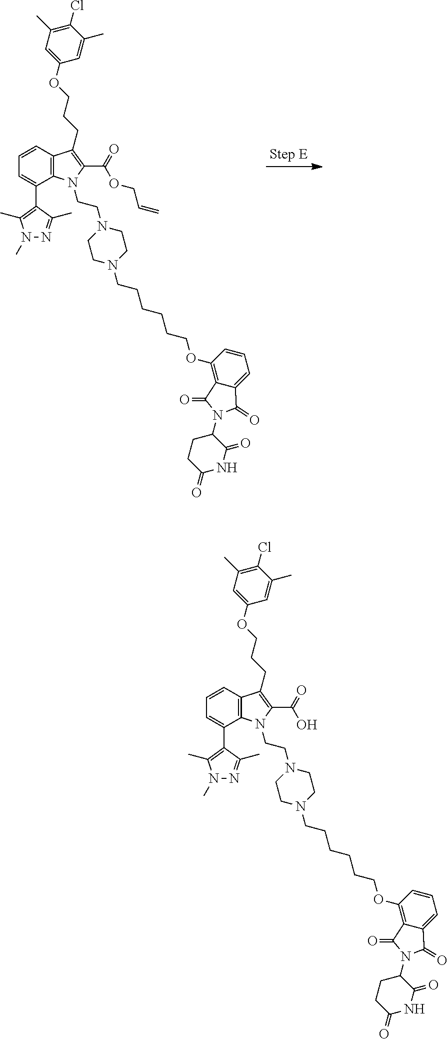 [SMILES: C=CCOC(=O)c1c(CCCOc2cc(C)c(Cl)c(C)c2)c2cccc(-c3c(C)nn(C)c3C)c2n1CCN1CCN(CCCCCCOc2cccc3c2C(=O)N(C2CCC(=O)NC2=O)C3=O)CC1 ; Cc1cc(OCCCc2c(C(=O)O)n(CCN3CCN(CCCCCCOc4cccc5c4C(=O)N(C4CCC(=O)NC4=O)C5=O)CC3)c3c(-c4c(C)nn(C)c4C)cccc23)cc(C)c1Cl]

To an ice cooled solution of allyl 3-(3-(4-chloro-3,5-dimethylphenoxy)propyl)-1-(2-(4-(6-((2-(2,6-dioxopiperidin-3-yl)-1,3-dioxoisoindolin-4-yl)oxy)hexyl)piperazin-1-yl)ethyl)-7-(1,3,5-trimethyl-1H-pyrazol-4-yl)-1H-indole-2-carboxylate (88 mg, crude) and tetrakis(triphenylphosphine)palladium(0) (10 mg, 0.01 mmol) in DCM (0.21 mL) was added morpholine (9 μL, 0.11 mmol). Reaction was stirred in RT overnight. Solvents were evaporated and crude product was purified with reverse phase flash chromatography (H₂O:MeCN+0.1% FA) to give 3-(3-(4-chloro-3,5-dimethylphenoxy)propyl)-1-(2-(4-(6-((2-(2,6-dioxopiperidin-3-yl)-1,3-dioxoisoindolin-4-yl)oxy)hexyl)piperazin-1-yl)ethyl)-7-(1,3,5-trimethyl-1H-pyrazol-4-yl)-1H-indole-2-carboxylic acid (1.58 mg, 0.002 mmol, 2% yield over five steps) as white solid.

LCMS (ESI+): m/z 934.1 [M+H]⁺

¹H NMR (500 MHz, DMSO) δ 11.09 (s, 1H), 7.79 (dd, J=8.5, 7.3 Hz, 1H), 7.63 (d, J=8.0 Hz, 1H), 7.49 (d, J=8.5 Hz, 1H), 7.43 (d, J=7.2 Hz, 1H), 7.08 (t, J=7.5 Hz, 1H), 6.89 (d, J=7.0 Hz, 1H), 6.72 (s, 2H), 5.07 (dd, J=12.8, 5.5 Hz, 1H), 4.42-4.30 (m, 1H), 4.27-4.13 (m, 3H), 3.97 (t, J=6.5 Hz, 2H), 3.72 (s, 3H), 3.12 (t, J=7.3 Hz, 2H), 2.88 (ddd, J=17.0, 13.9, 5.5 Hz, 1H), 2.62-2.56 (m, 1H), 2.56-2.51 (m, 2H), 2.25 (s, 6H), 2.24-2.07 (m, 10H), 2.06 (s, 3H), 2.05-1.99 (m, 3H), 1.92 (s, 3H), 1.73 (dt, J=14.1, 6.4 Hz, 2H), 1.50-1.38 (m, 3H), 1.38-1.30 (m, 4H).

### Example 31: 3-(3-(4-chloro-3,5-dimethylphenoxy)propyl)-1-(2-(4-(3-(3-((2-(2,6-dioxopiperidin-3-yl)-1,3-dioxoisoindolin-4-yl)amino)propoxy)propyl)piperazin-1-yl)ethyl)-7-(1,3,5-trimethyl-1H-pyrazol-4-yl)-1H-indole-2-carboxylic acid (236)

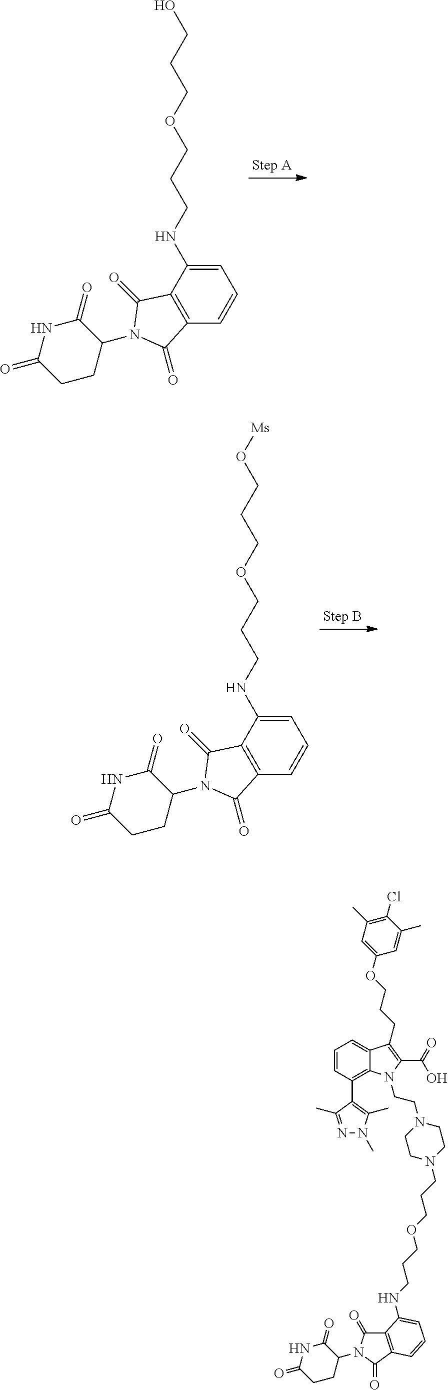 [SMILES: O=C1CCC(N2C(=O)c3cccc(NCCCOCCCO)c3C2=O)C(=O)N1 ; CS(=O)(=O)OCCCOCCCNc1cccc2c1C(=O)N(C1CCC(=O)NC1=O)C2=O ; Cc1cc(OCCCc2c(C(=O)O)n(CCN3CCN(CCCOCCCNc4cccc5c4C(=O)N(C4CCC(=O)NC4=O)C5=O)CC3)c3c(-c4c(C)nn(C)c4C)cccc23)cc(C)c1Cl]

### Step A

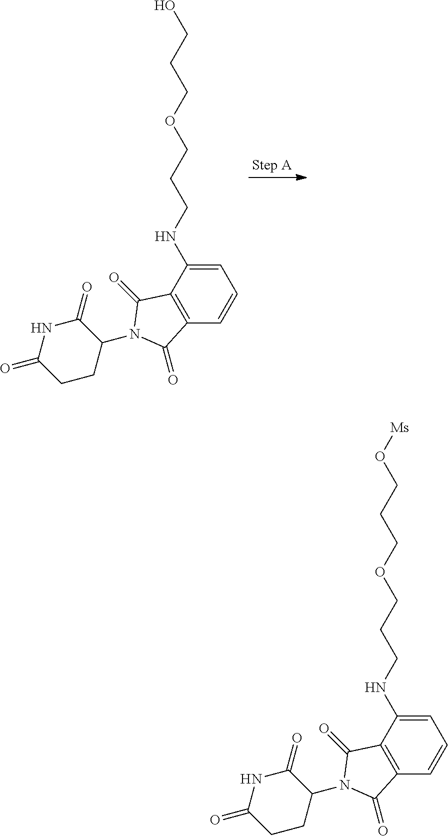 [SMILES: O=C1CCC(N2C(=O)c3cccc(NCCCOCCCO)c3C2=O)C(=O)N1 ; CS(=O)(=O)OCCCOCCCNc1cccc2c1C(=O)N(C1CCC(=O)NC1=O)C2=O]

### General Procedure 1:

Starting material and Et₃N were dissolved in DCM and cooled down to 0° C. A solution of MsCl in DCM was added dropwise in that temperature. The mixture was stirred at RT for 18 h and H₂O was added. The layers were separated and the aqueous layer was extracted with DCM. The combined organic extracts were washed with H₂O, dried over Na₂SO₄, and concentrated to obtain crude of methanesulfonate, which was used for next step without further purification.

### Step B

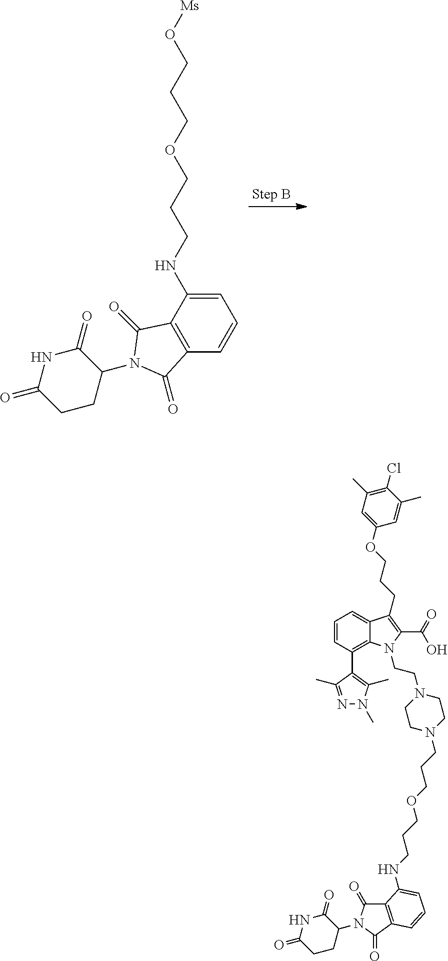 [SMILES: CS(=O)(=O)OCCCOCCCNc1cccc2c1C(=O)N(C1CCC(=O)NC1=O)C2=O ; Cc1cc(OCCCc2c(C(=O)O)n(CCN3CCN(CCCOCCCNc4cccc5c4C(=O)N(C4CCC(=O)NC4=O)C5=O)CC3)c3c(-c4c(C)nn(C)c4C)cccc23)cc(C)c1Cl]

To a solution of 3-[3-(4-chloro-3,5-dimethylphenoxy)propyl]-1-[2-(piperazin-1-yl)ethyl]-7-(1,3,5-trimethyl-1H-pyrazol-4-yl)-1H-indole-2-carboxylic acid hydrochloride (1.00 g, 1.63 mmol) in DMSO (10 mL) were added DIPEA (2.05 mL, 11.73 mmol) and 3-(3-((2-(2,6-dioxopiperidin-3-yl)-1,3-dioxoisoindolin-4-yl)amino)propoxy)propyl methanesulfonate (0.91 g, 1.95 mmol). The reaction mixture was stirred at 80° C. Upon completion, the mixture was poured into water, extracted with CHCl₃, and the combined organic layers were dried over anhydrous Na₂SO₄ and evaporated under reduced pressure. Crude product was purified by column chromatography on silica gel to afford 3-(3-(4-chloro-3,5-dimethylphenoxy)propyl)-1-(2-(4-(3-(3-((2-(2,6-dioxopiperidin-3-yl)-1,3-dioxoisoindolin-4-yl)amino)propoxy)propyl)piperazin-1-yl)ethyl)-7-(1,3,5-trimethyl-1H-pyrazol-4-yl)-1H-indole-2-carboxylic acid (36 mg, 0.038 mmol, 2% over steps A and B) as yellow solid.

LCMS (ESI+): m/z 949.4 [M+H]⁺

¹H NMR (400 MHz, CD₃OD) δ 7.62 (d, J=7.9 Hz, 1H), 7.50 (t, J=7.7 Hz, 1H), 7.09 (t, J=7.6 Hz, 1H), 7.04-6.95 (m, 2H), 6.88 (d, J=7.1 Hz, 1H), 6.63 (s, 2H), 5.05 (dd, J=12.3, 5.2 Hz, 1H), 4.38 (s, 2H), 3.95 (t, J=6.6 Hz, 2H), 3.79 (d, J=3.2 Hz, 3H), 3.54 (t, J=5.7 Hz, 2H), 3.44 (t, J=6.3 Hz, 2H), 3.37 (t, J=6.2 Hz, 2H), 3.20 (t, J=7.3 Hz, 2H), 2.85-2.68 (m, 8H), 2.51 (s, 4H), 2.41 (s, 2H), 2.26 (s, 6H), 2.11 (d, J=4.9 Hz, 6H), 2.04 (s, 3H), 1.89 (t, J=6.0 Hz, 3H), 1.85-1.76 (m, 2H).

### Example 32. 3-(3-(4-chloro-3,5-dimethylphenoxy)propyl)-1-(2-(4-(3-(2-(2-((2-(2,6-dioxopiperidin-3-yl)-1,3-dioxoisoindolin-4-yl)amino)ethoxy)ethoxy)propyl)piperazin-1-yl)ethyl)-7-(1,3,5-trimethyl-1H-pyrazol-4-yl)-1H-indole-2-carboxylic acid (237)

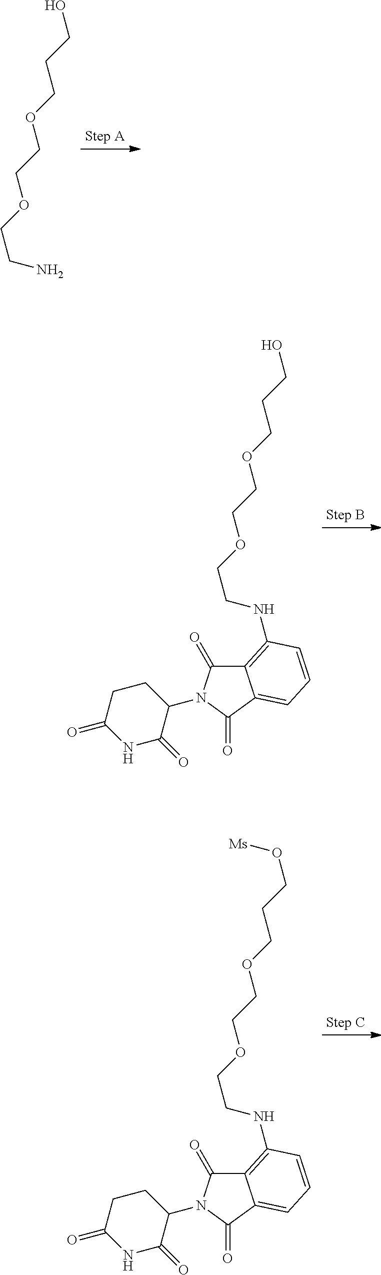 [SMILES: NCCOCCOCCCO ; O=C1CCC(N2C(=O)c3cccc(NCCOCCOCCCO)c3C2=O)C(=O)N1 ; CS(=O)(=O)OCCCOCCOCCNc1cccc2c1C(=O)N(C1CCC(=O)NC1=O)C2=O]

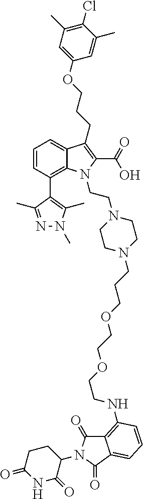 [SMILES: Cc1cc(OCCCc2c(C(=O)O)n(CCN3CCN(CCCOCCOCCNc4cccc5c4C(=O)N(C4CCC(=O)NC4=O)C5=O)CC3)c3c(-c4c(C)nn(C)c4C)cccc23)cc(C)c1Cl]

### Step A

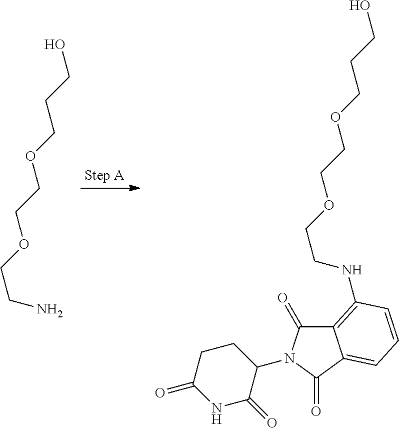 [SMILES: NCCOCCOCCCO ; O=C1CCC(N2C(=O)c3cccc(NCCOCCOCCCO)c3C2=O)C(=O)N1]

To a stirred solution of 2-(2,6-dioxopiperidin-3-yl)-4-fluoroisoindoline-1,3-dione (38.20 g, 138.31 mmol) in DMF (300 mL) were added DIPEA (48.31 mL, 276.61 mmol) and 3-(2-(2-aminoethoxy)ethoxy)propan-1-ol (22.57 g, 138.31 mmol). The reaction mixture was heated to 100° C. overnight, then cooled to room temperature, and taken up in EtOAc (300 mL) and water (300 mL). The organic layer was washed with brine, dried over Na₂SO₄, and concentrated in vacuo. The residue was purified by flash column chromatography on silica gel to give 2-(2,6-dioxopiperidin-3-yl)-4-((2-(2-(3-hydroxypropoxy)ethoxy)ethyl)amino)isoindoline-1,3-dione (9.80 g, 23.389 mmol, 17%).

### Step B

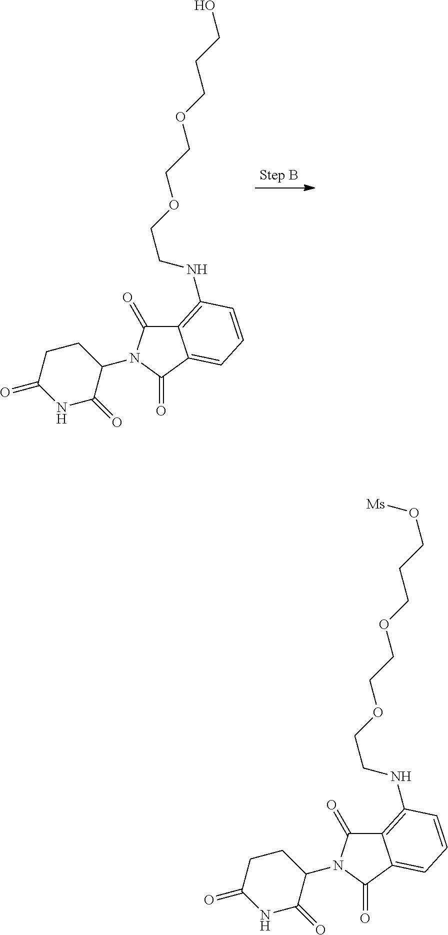 [SMILES: O=C1CCC(N2C(=O)c3cccc(NCCOCCOCCCO)c3C2=O)C(=O)N1 ; CS(=O)(=O)OCCCOCCOCCNc1cccc2c1C(=O)N(C1CCC(=O)NC1=O)C2=O]

3-(2-(2-((2-(2,6-dioxopiperidin-3-yl)-1,3-dioxoisoindolin-4-yl)amino)ethoxy)ethoxy)propyl methanesulfonate was prepared according to general procedure 1 using 2-(2,6-dioxopiperidin-3-yl)-4-((2-(2-(3-hydroxypropoxy)ethoxy)ethyl)amino)isoindoline-1,3-dione (0.7 g, 1.68 mmol), Et₃N (1 mL, 7 mmol) and MsCl (0.23 g, 2 mmol) to obtain 0.83 g (crude) of title compound.

### Step C

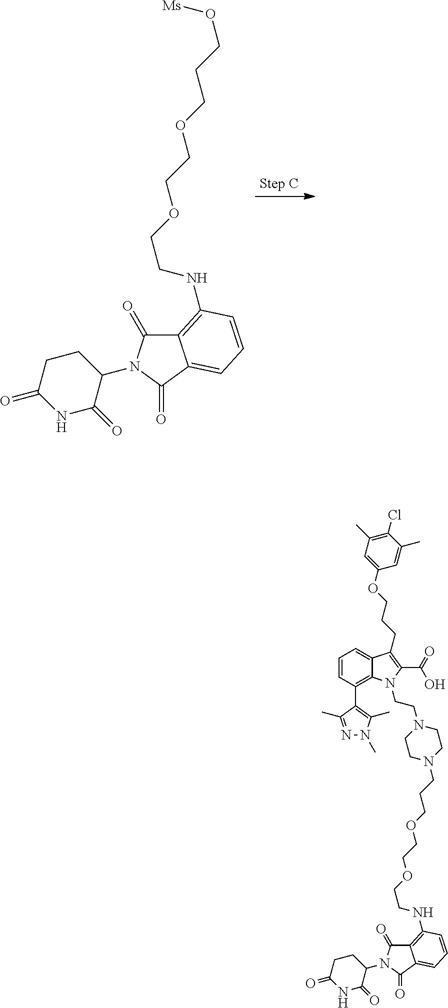 [SMILES: CS(=O)(=O)OCCCOCCOCCNc1cccc2c1C(=O)N(C1CCC(=O)NC1=O)C2=O ; Cc1cc(OCCCc2c(C(=O)O)n(CCN3CCN(CCCOCCOCCNc4cccc5c4C(=O)N(C4CCC(=O)NC4=O)C5=O)CC3)c3c(-c4c(C)nn(C)c4C)cccc23)cc(C)c1Cl]

To a solution of 3-[3-(4-chloro-3,5-dimethylphenoxy)propyl]-1-[2-(piperazin-1-yl)ethyl]-7-(1,3,5-trimethyl-1H-pyrazol-4-yl)-1H-indole-2-carboxylic acid hydrochloride (0.52 g, crude) in DMSO (5 mL) were added DIPEA (0.75 g, 5.80 mmol) and 3-(2-(2-((2-(2,6-dioxopiperidin-3-yl)-1,3-dioxoisoindolin-4-yl)amino)ethoxy)ethoxy)propyl methanesulfonate (0.51 g, 1.03 mmol). The reaction mixture was left stirred at 80° C. until accepted conversion of target compound was observed. The reaction mixture was poured in water, extracted with chloroform, dried over Na₂SO₄, and concentrated under reduced pressure. The crude product was purified by column chromatography to obtain 3-(3-(4-chloro-3,5-dimethylphenoxy)propyl)-1-(2-(4-(3-(2-(2-((2-(2,6-dioxopiperidin-3-yl)-1,3-dioxoisoindolin-4-yl)amino)ethoxy)ethoxy)propyl)piperazin-1-yl)ethyl)-7-(1,3,5-trimethyl-1H-pyrazol-4-yl)-1H-indole-2-carboxylic acid (0.016 g, 0.016 mmol, 1.9%) as yellow solid.

LCMS (ESI+): m/z 978.8 [M+H]⁺

¹H NMR (400 MHz, CDCl₃) δ 7.68-7.56 (m, 1H), 7.47 (td, J=7.8, 3.8 Hz, 1H), 7.17-7.02 (m, 2H), 6.97-6.81 (m, 2H), 6.60 (d, J=3.7 Hz, 2H), 6.57-6.47 (m, 1H), 5.16-4.82 (m, 1H), 4.80-4.17 (m, 2H), 4.05-3.88 (m, 2H), 3.77 (d, J=17.4 Hz, 3H), 3.70 (t, J=5.2 Hz, 2H), 3.65-3.57 (m, 2H), 3.57-3.48 (m, 2H), 3.46-3.32 (m, 4H), 3.27-3.13 (m, 2H), 2.94-2.63 (m, 6H), 2.61-2.30 (m, 8H), 2.30-2.24 (m, 6H), 2.24-1.93 (m, 10H), 1.93-1.67 (m, 2H).

### Example 33. 3-(3-(4-chloro-3,5-dimethylphenoxypropyl)-1-(3-(2-((2-(2,6-dioxopiperidin-3-yl)-1,3-dioxoisoindolin-4-yl)oxy)ethoxy)propyl)-7-(1,3,5-trimethyl-1H-pyrazol-4-yl)-1H-indole-2-carboxylic acid (238)

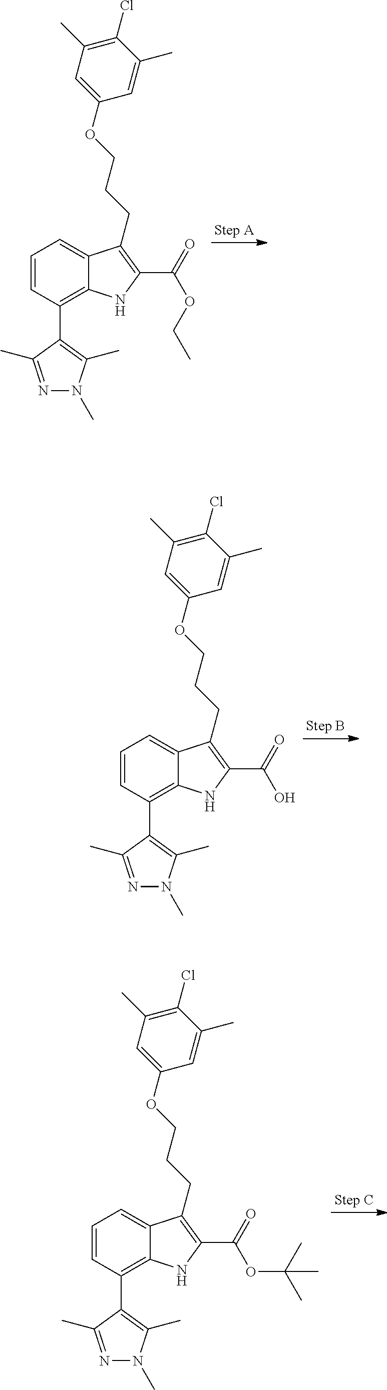 [SMILES: CCOC(=O)c1[nH]c2c(-c3c(C)nn(C)c3C)cccc2c1CCCOc1cc(C)c(Cl)c(C)c1 ; Cc1cc(OCCCc2c(C(=O)O)[nH]c3c(-c4c(C)nn(C)c4C)cccc23)cc(C)c1Cl ; Cc1cc(OCCCc2c(C(=O)OC(C)(C)C)[nH]c3c(-c4c(C)nn(C)c4C)cccc23)cc(C)c1Cl]

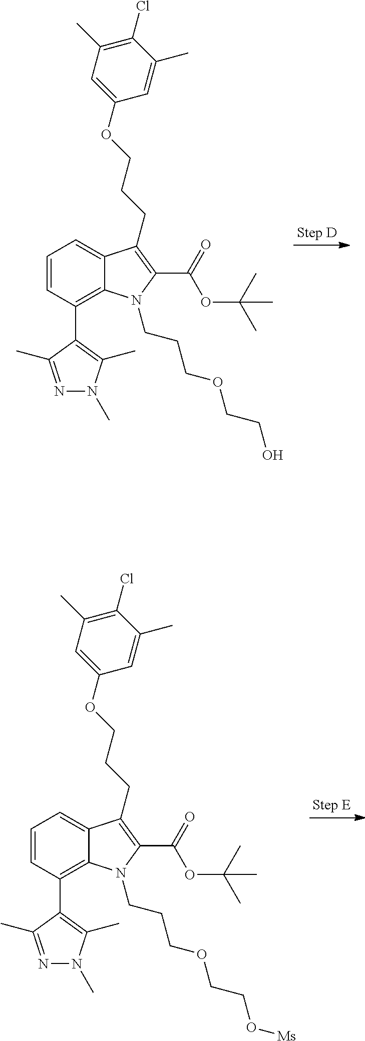

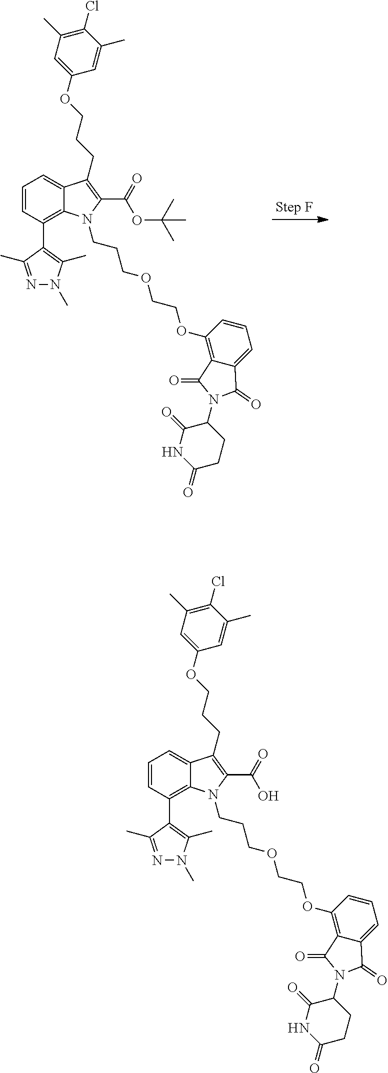 [SMILES: Cc1cc(OCCCc2c(C(=O)OC(C)(C)C)n(CCCOCCOc3cccc4c3C(=O)N(C3CCC(=O)NC3=O)C4=O)c3c(-c4c(C)nn(C)c4C)cccc23)cc(C)c1Cl ; Cc1cc(OCCCc2c(C(=O)O)n(CCCOCCOc3cccc4c3C(=O)N(C3CCC(=O)NC3=O)C4=O)c3c(-c4c(C)nn(C)c4C)cccc23)cc(C)c1Cl]

### Step A

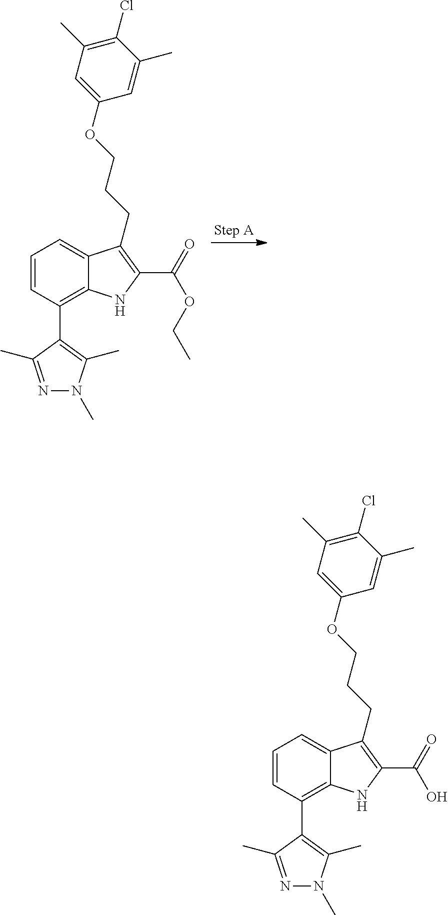 [SMILES: CCOC(=O)c1[nH]c2c(-c3c(C)nn(C)c3C)cccc2c1CCCOc1cc(C)c(Cl)c(C)c1 ; Cc1cc(OCCCc2c(C(=O)O)[nH]c3c(-c4c(C)nn(C)c4C)cccc23)cc(C)c1Cl]

To a solution of compound ethyl 3-(3-(4-chloro-3,5-dimethylphenoxy)propyl)-7-(1,3,5-trimethyl-1H-pyrazol-4-yl)-1H-indole-2-carboxylate (7 g, 14.2 mmol) in THE (50 mL) and MeOH (50 mL) was added a solution of NaOH (2.3 g, 57.5 mmol) in water (20 mL). The reaction mixture was heated under reflux for 2 h, cooled to room temperature and concentrated in vacuo. The residue was dissolved in water and washed with EtOAc. The water phase was acidified to neutral pH and extracted with DCM. The organic layer was dried over Na₂SO₄, filtered and concentrated in vacuo to give 3-(3-(4-chloro-3,5-dimethylphenoxy)propyl)-7-(1,3,5-trimethyl-1H-pyrazol-4-yl)-1H-indole-2-carboxylic acid (5.1 g, 11 mmol), which was used to next step without further purification.

### Step B

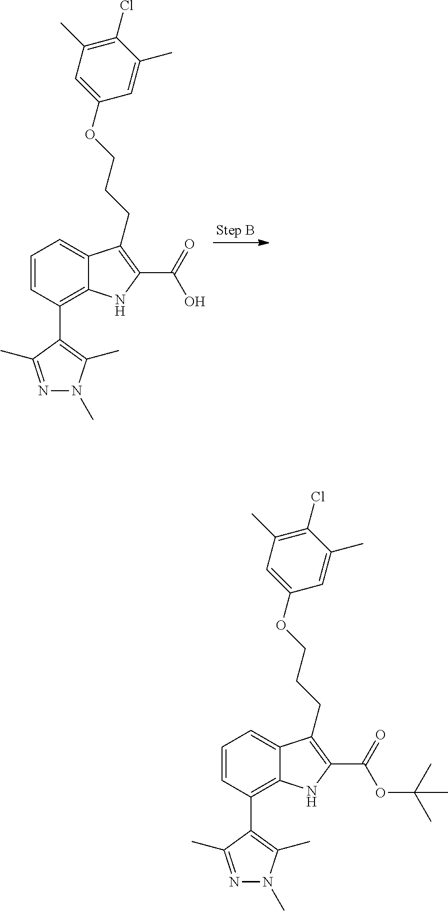 [SMILES: Cc1cc(OCCCc2c(C(=O)O)[nH]c3c(-c4c(C)nn(C)c4C)cccc23)cc(C)c1Cl ; Cc1cc(OCCCc2c(C(=O)OC(C)(C)C)[nH]c3c(-c4c(C)nn(C)c4C)cccc23)cc(C)c1Cl]

To a solution of 3-(3-(4-chloro-3,5-dimethylphenoxy)propyl)-7-(1,3,5-trimethyl-1H-pyrazol-4-yl)-1H-indole-2-carboxylic acid from the previous step (5.1 g, 11 mmol) in DCM (100 mL) was added tert-butyl 2,2,2-trichloroacetimidate (7.16 g, 33 mmol) at 20° C. The reaction mixture was stirred for 48 h at 20° C. The solution was concentrated in vacuo and the crude residue was purified by flash chromatography to give tert-butyl 3-(3-(4-chloro-3,5-dimethylphenoxy)propyl)-7-(1,3,5-trimethyl-1H-pyrazol-4-yl)-1H-indole-2-carboxylate (2.2 g, 4.2 mmol, 30% over two steps) as a solid.

### Step C

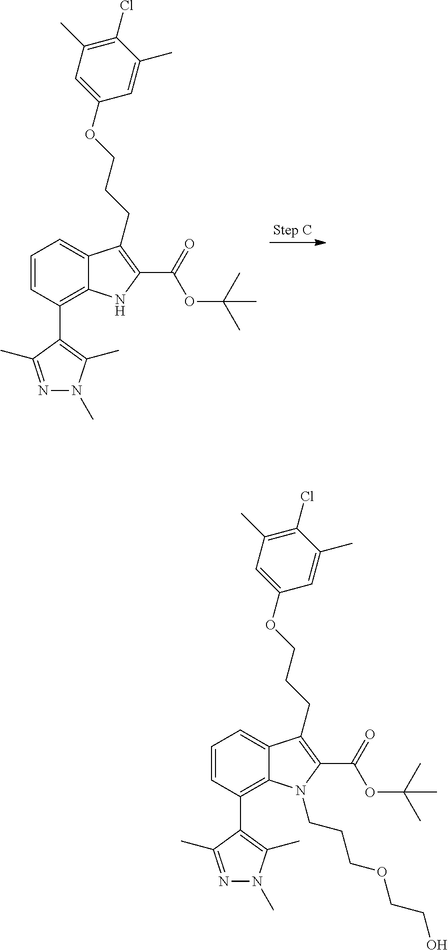 [SMILES: Cc1cc(OCCCc2c(C(=O)OC(C)(C)C)[nH]c3c(-c4c(C)nn(C)c4C)cccc23)cc(C)c1Cl ; Cc1cc(OCCCc2c(C(=O)OC(C)(C)C)n(CCCOCCO)c3c(-c4c(C)nn(C)c4C)cccc23)cc(C)c1Cl]

To a solution of the tert-butyl 3-(3-(4-chloro-3,5-dimethylphenoxy)propyl)-7-(1,3,5-trimethyl-1H-pyrazol-4-yl)-1H-indole-2-carboxylate (100 mg, 0.192 mmol) in DMF (6 mL), 2-(3-chloropropoxy)ethanol (265 mg, 1.92 mmol), KI (64 mg, 0.38 mmol), and K₂CO₃ (79 mg, 0.57 mmol) were added. Subsequently, the reaction mixture was stirring at 70° C. for 24 h. After the reaction was complete (monitored by TLC), DMF was evaporated and the resultant residue was partitioned between EtOAc and H₂O. The organic layer was further washed with brine, dried over Na₂SO₄, filtered and evaporated. The final product was purified on preparative TLC (hexane:EtOAc:MeOH, 50:47:3) to obtain tert-butyl 3-(3-(4-chloro-3,5-dimethylphenoxy)propyl)-1-(3-(2-hydroxyethoxy)propyl)-7-(1,3,5-trimethyl-1H-pyrazol-4-yl)-1H-indole-2-carboxylate (36.00 mg, 0.058 mmol, 30%).

LCMS (ESI+): m/z 624.3 [M+H]⁺

### Step D

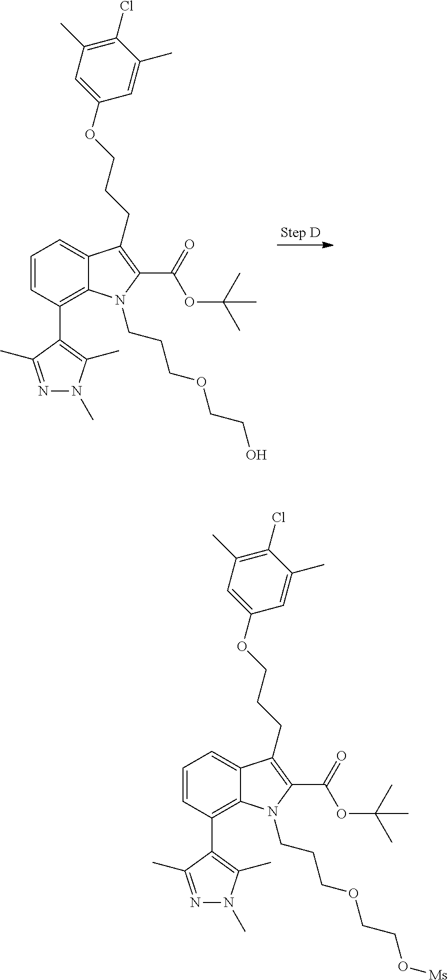 [SMILES: Cc1cc(OCCCc2c(C(=O)OC(C)(C)C)n(CCCOCCO)c3c(-c4c(C)nn(C)c4C)cccc23)cc(C)c1Cl ; Cc1cc(OCCCc2c(C(=O)OC(C)(C)C)n(CCCOCCOS(C)(=O)=O)c3c(-c4c(C)nn(C)c4C)cccc23)cc(C)c1Cl]

To a solution of tert-butyl 3-(3-(4-chloro-3,5-dimethylphenoxy)propyl)-1-(3-(2-hydroxyethoxy)propyl)-7-(1,3,5-trimethyl-1H-pyrazol-4-yl)-1H-indole-2-carboxylate (30 mg, 0.05 mmol) in dry DCM (6 mL) Et₃N (19 mg, 0.19 mmol) was added under argon atmosphere and the mixture was cooled to 0° C. Subsequently, methanesulfonyl chloride (28 mg, 0.24 mmol) was added dropwise at 0° C., followed by stirring of the mixture at room temperature for 24 h. After the reaction was complete (monitored by TLC), MeOH (20 mL) was added to the mixture carefully. The volatiles were then evaporated and the resultant residue was partitioned between EtOAc and H₂O. The organic layer was further washed with brine, dried over Na₂SO₄, filtered and evaporated to give tert-butyl 3-(3-(4-chloro-3,5-dimethylphenoxy)propyl)-1-(3-(2-((methylsulfonyl)oxy)ethoxy)propyl)-7-(1,3,5-trimethyl-1H-pyrazol-4-yl)-1H-indole-2-carboxylate (30.00 mg, crude) which was used directly to the next step.

### Step E

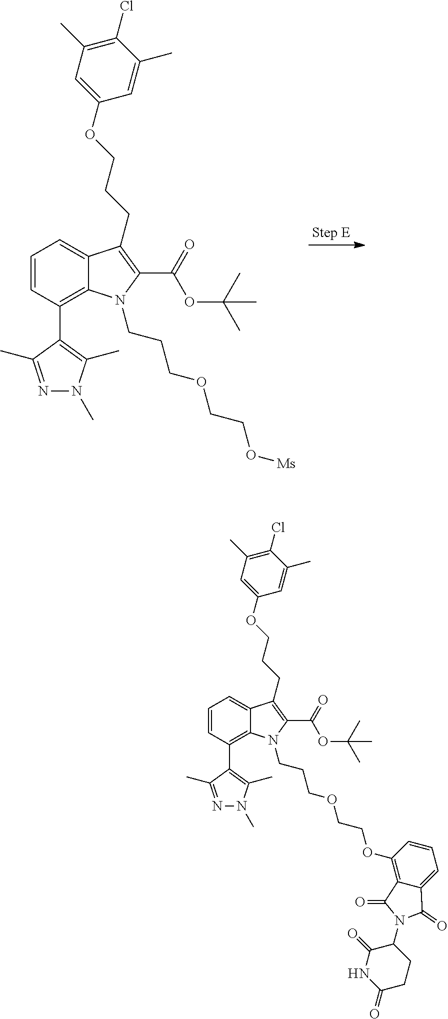 [SMILES: Cc1cc(OCCCc2c(C(=O)OC(C)(C)C)n(CCCOCCOS(C)(=O)=O)c3c(-c4c(C)nn(C)c4C)cccc23)cc(C)c1Cl ; Cc1cc(OCCCc2c(C(=O)OC(C)(C)C)n(CCCOCCOc3cccc4c3C(=O)N(C3CCC(=O)NC3=O)C4=O)c3c(-c4c(C)nn(C)c4C)cccc23)cc(C)c1Cl]

To a solution of the tert-butyl 3-(3-(4-chloro-3,5-dimethylphenoxy)propyl)-1-(3-(2-((methylsulfonyl)oxy)ethoxy)propyl)-7-(1,3,5-trimethyl-1H-pyrazol-4-yl)-1H-indole-2-carboxylate (30 mg, crude) and 2-(2,6-dioxopiperidin-3-yl)-4-hydroxyisoindoline-1,3-dione (15 mg, 0.056 mmol) in DMF (0.95 mL), KI (8 mg, 0.05 mmol) followed by KHCO₃ (11 mg, 0.11 mmol). The mixture was stirred in 60° C. for 24 h. Additional portion of K₂CO₃ (9 mg, 0.06 mmol) and KI (8 mg, 0.05 mmol) was added after this time. The reaction mixture was stirred in 60° C. for the next 24 h. The crude was purified on preparative TLC (hexane:EtOAc:MeOH, 50:42:8) to obtain tert-butyl 3-(3-(4-chloro-3,5-dimethylphenoxy)propyl)-1-(3-(2-((2-(2,6-dioxopiperidin-3-yl)-1,3-dioxoisoindolin-4-yl)oxy)ethoxy)propyl)-7-(1,3,5-trimethyl-1H-pyrazol-4-yl)-1H-indole-2-carboxylate (3.00 mg, 0.003 mmol, 6% over two steps).

### Step F

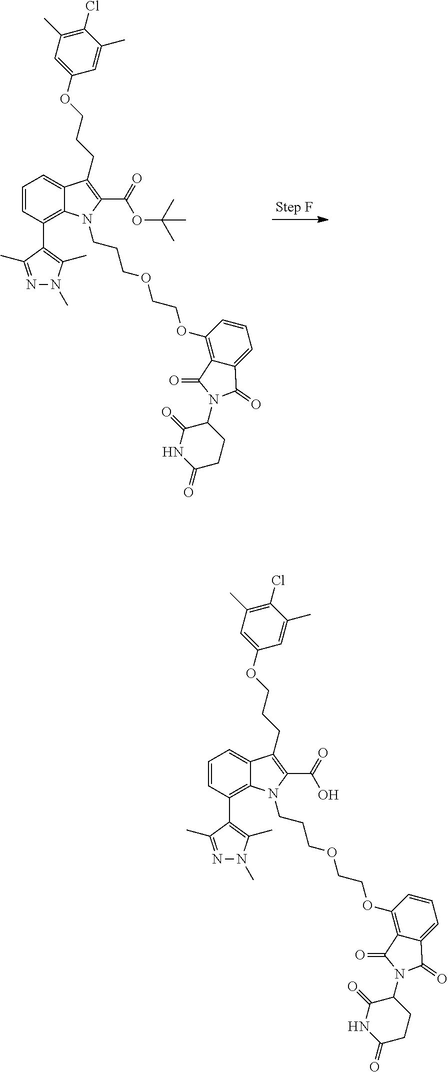 [SMILES: Cc1cc(OCCCc2c(C(=O)OC(C)(C)C)n(CCCOCCOc3cccc4c3C(=O)N(C3CCC(=O)NC3=O)C4=O)c3c(-c4c(C)nn(C)c4C)cccc23)cc(C)c1Cl ; Cc1cc(OCCCc2c(C(=O)O)n(CCCOCCOc3cccc4c3C(=O)N(C3CCC(=O)NC3=O)C4=O)c3c(-c4c(C)nn(C)c4C)cccc23)cc(C)c1Cl]

Tert-butyl 3-(3-(4-chloro-3,5-dimethylphenoxy)propyl)-1-(3-(2-((2-(2,6-dioxopiperidin-3-yl)-1,3-dioxoisoindolin-4-yl)oxy)ethoxy)propyl)-7-(1,3,5-trimethyl-1H-pyrazol-4-yl)-1H-indole-2-carboxylate (3 mg, 0.003 mmol) was dissolved in 4 M HCl in dioxane (17 μL, 0.068 mmol). Subsequently, the mixture was stirring at room temperature for 24 h. After the reaction was completed (monitored by LCMS), solvent was evaporated and the compound was purified twice on preparative TLC (hexane:EtOAc:EtOH 50:43:7). The final product 3-(3-(4-chloro-3,5-dimethylphenoxy)propyl)-1-(3-(2-((2-(2,6-dioxopiperidin-3-yl)-1,3-dioxoisoindolin-4-yl)oxy)ethoxy)propyl)-7-(1,3,5-trimethyl-1H-pyrazol-4-yl)-1H-indole-2-carboxylic acid (2.00 mg, 0.002 mmol, 71%) was obtained as white solid.

LCMS (ESI+): m/z 824.2 [M+H]⁺

¹H NMR (500 MHz, DMSO) δ 11.09 (s, 1H), 7.80 (dd, J=8.6, 7.3 Hz, 1H), 7.51 (d, J=8.5 Hz, 1H), 7.45 (d, J=7.2 Hz, 1H), 7.31 (dt, J=7.3, 1.2 Hz, 1H), 7.08 (t, J=7.5 Hz, 1H), 6.94 (ddd, J=7.7, 4.1, 1.4 Hz, 1H), 6.69 (d, J=3.2 Hz, 2H), 5.11-5.03 (m, 1H), 4.36-4.22 (m, 2H), 3.92-3.75 (m, 2H), 3.71-3.65 (m, 3H), 3.65-3.59 (m, 2H), 3.27-3.20 (m, 1H), 3.15-3.03 (m, 2H), 2.87 (ddd, J=17.1, 13.8, 5.4 Hz, 1H), 2.62-2.53 (m, 1H), 2.54-2.50 (m, 1H), 2.25 (s, 6H), 2.10-1.80 (m, 10H), 1.55-1.32 (m, 4H).

### Example 34. 3-(3-(4-chloro-3,5-dimethylphenoxy)propyl)-1-(2-(4-(2-(((2-(2,6-dioxopiperidin-3-yl)-1-oxoisoindolin-5-yl)methyl)amino)-2-oxoethyl)piperazin-1-yl)ethyl)-7-(1,3,5-trimethyl-1H-pyrazol-4-yl)-1H-indole-2-carboxylic acid (240)

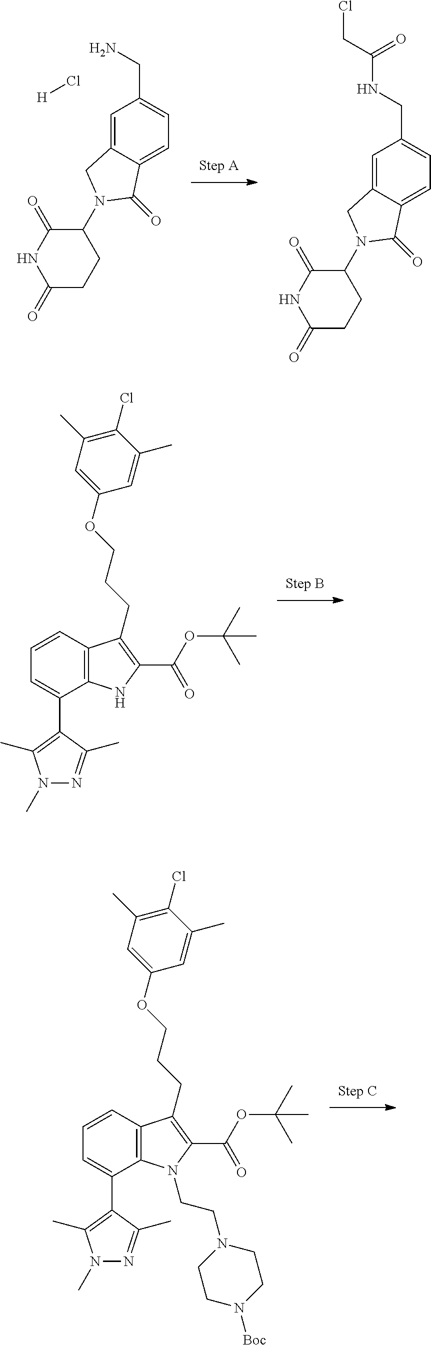 [SMILES: NCc1ccc2c(c1)CN(C1CCC(=O)NC1=O)C2=O ; Cl ; O=C(CCl)NCc1ccc2c(c1)CN(C1CCC(=O)NC1=O)C2=O ; Cc1cc(OCCCc2c(C(=O)OC(C)(C)C)[nH]c3c(-c4c(C)nn(C)c4C)cccc23)cc(C)c1Cl ; Cc1cc(OCCCc2c(C(=O)OC(C)(C)C)n(CCN3CCN(C(=O)OC(C)(C)C)CC3)c3c(-c4c(C)nn(C)c4C)cccc23)cc(C)c1Cl]

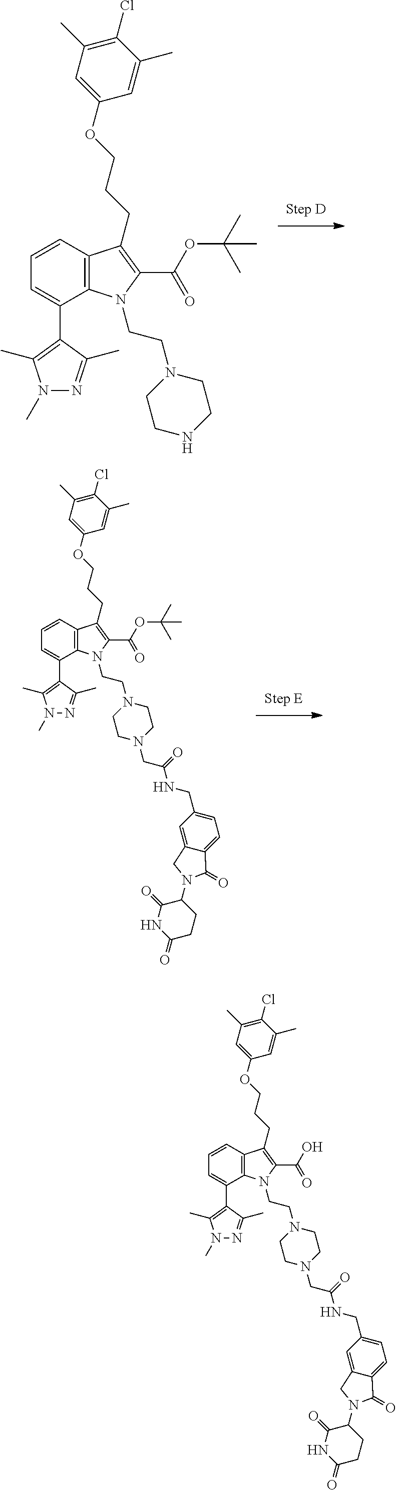

### Step A

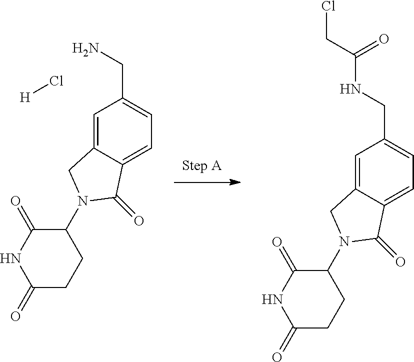 [SMILES: NCc1ccc2c(c1)CN(C1CCC(=O)NC1=O)C2=O ; Cl ; O=C(CCl)NCc1ccc2c(c1)CN(C1CCC(=O)NC1=O)C2=O]

To the solution of 3-[5-(aminomethyl)-1-oxo-2,3-dihydro-1H-isoindol-2-yl]piperidine-2,6-dione hydrochloride (50.0 mg, 0.161 mmol) in DMF (1.5 mL) was added DIPEA (0.042 mL, 0.242 mmol), followed by chloroacetyl chloride (0.014 mL, 0.178 mmol). Mixture was stirred for overnight at RT. DMF was evaporated and the resultant residue was partitioned between EtOAc and H₂O. The organic layer was further washed with brine, dried over Na₂SO₄, filtered and evaporated. 2-Chloro-N-((2-(2,6-dioxopiperidin-3-yl)-1-oxoisoindolin-5-yl)methyl)acetamide (60.0 mg, 0.161 mmol, 99.8%) was obtained as orangish solid.

LCMS (ESI+): m/z 349.9 [M+H]⁺

### Step B

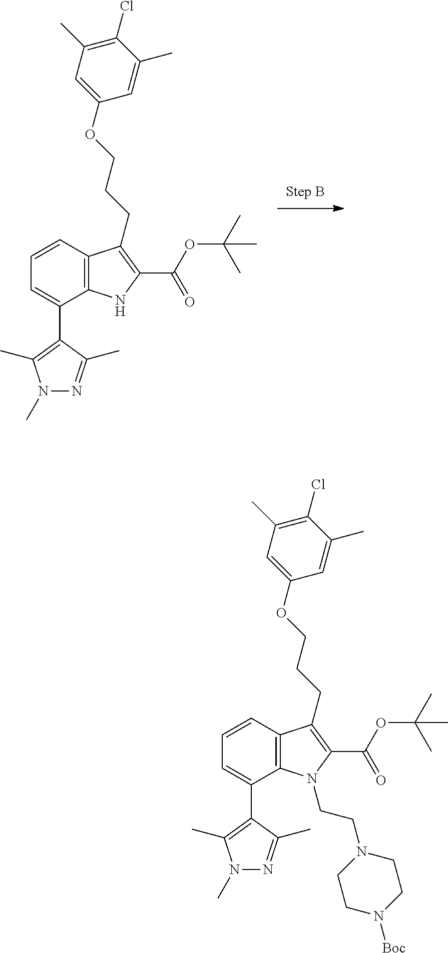 [SMILES: Cc1cc(OCCCc2c(C(=O)OC(C)(C)C)[nH]c3c(-c4c(C)nn(C)c4C)cccc23)cc(C)c1Cl ; Cc1cc(OCCCc2c(C(=O)OC(C)(C)C)n(CCN3CCN(C(=O)OC(C)(C)C)CC3)c3c(-c4c(C)nn(C)c4C)cccc23)cc(C)c1Cl]

Tert-butyl 3-(3-(4-chloro-3,5-dimethylphenoxy)propyl)-7-(1,3,5-trimethyl-1H-pyrazol-4-yl)-1H-indole-2-carboxylate (0.200 g, 0.383 mmol) and tert-butyl 4-(2-chloroethyl)piperazine-1-carboxylate (0.095 g, 0.383 mmol) were dissolved in DMF (3.8 mL). Subsequently, KI (0.064 g, 0.383 mmol) and Cs₂CO₃ (0.374 g, 1.149 mmol) were added. The reaction mixture was stirring at 70° C. for 24 h. DMF was evaporated and the resultant residue was partitioned between EtOAc and H₂O. The organic layer was further washed with brine, dried over Na₂SO₄, filtered and evaporated. Tert-butyl 1-(2-(4-(tert-butoxycarbonyl)piperazin-1-yl)ethyl)-3-(3-(4-chloro-3,5-dimethylphenoxy)propyl)-7-(1,3,5-trimethyl-1H-pyrazol-4-yl)-1H-indole-2-carboxylate (214 mg, crude) was used to the next step without purification.

LCMS (ESI+): m/z 734.2 [M+H]⁺

### Step C

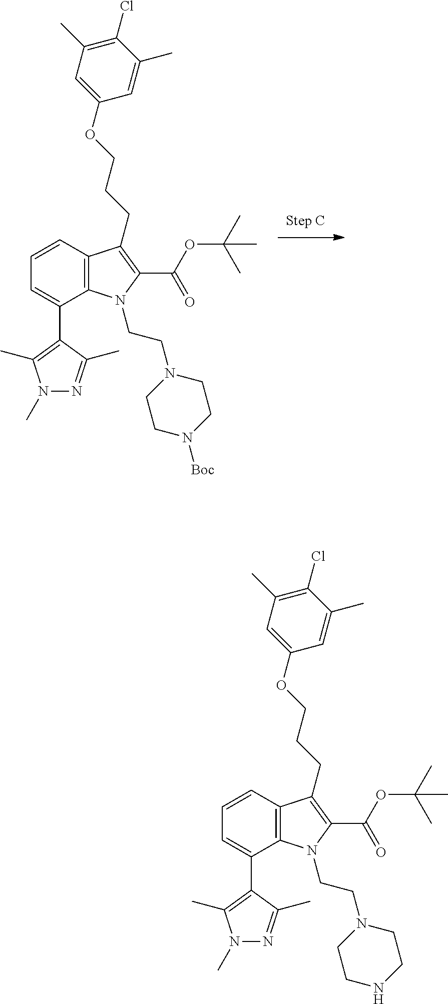 [SMILES: Cc1cc(OCCCc2c(C(=O)OC(C)(C)C)n(CCN3CCN(C(=O)OC(C)(C)C)CC3)c3c(-c4c(C)nn(C)c4C)cccc23)cc(C)c1Cl ; Cc1cc(OCCCc2c(C(=O)OC(C)(C)C)n(CCN3CCNCC3)c3c(-c4c(C)nn(C)c4C)cccc23)cc(C)c1Cl]

Tert-butyl 1-(2-(4-(tert-butoxycarbonyl)piperazin-1-yl)ethyl)-3-(3-(4-chloro-3,5-dimethylphenoxy)propyl)-7-(1,3,5-trimethyl-1H-pyrazol-4-yl)-1H-indole-2-carboxylate (214.0 mg, crude) was dissolved in THE (5.8 mL) and 4M HCl in dioxane (0.506 mL, 14.57 mmol) was added at 0° C. and the mixture stirred for 24 h. The reaction mixture was quenched by cold 0.1M NaOH solution (10 mL) and extracted several times with EtOAc and one time with acetonitrile. The combined organics were dried over Na₂SO₄ and concentrated in vacuo. Desired product was purified using flash chromatography (SiO₂, 20% MeOH in DCM). Tert-butyl 3-(3-(4-chloro-3,5-dimethylphenoxy)propyl)-1-(2-(piperazin-1-yl)ethyl)-7-(1,3,5-trimethyl-1H-pyrazol-4-yl)-1H-indole-2-carboxylate (139.0 mg, 0.210 mmol, 55% over two steps) was obtained as orange oil.

LCMS (ESI+): m/z 634.3 [M+H]⁺

### Step D

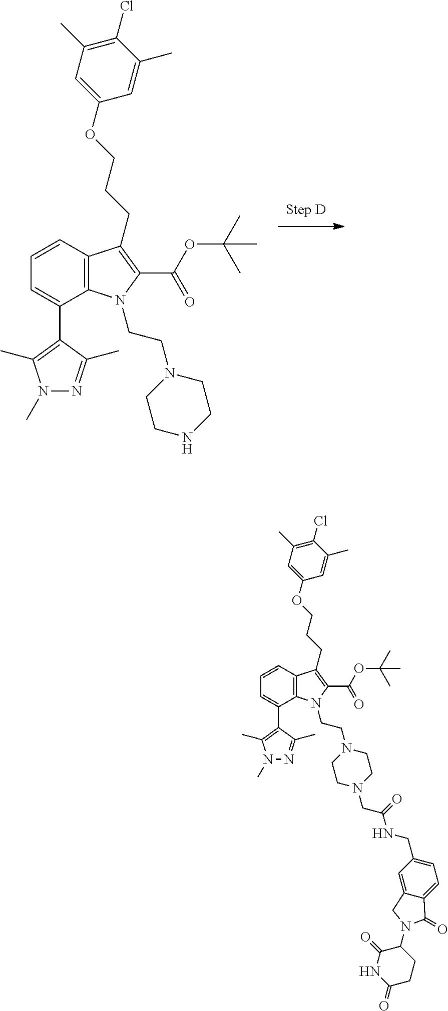 [SMILES: Cc1cc(OCCCc2c(C(=O)OC(C)(C)C)n(CCN3CCNCC3)c3c(-c4c(C)nn(C)c4C)cccc23)cc(C)c1Cl ; Cc1cc(OCCCc2c(C(=O)OC(C)(C)C)n(CCN3CCN(CC(=O)NCc4ccc5c(c4)CN(C4CCC(=O)NC4=O)C5=O)CC3)c3c(-c4c(C)nn(C)c4C)cccc23)cc(C)c1Cl]

Tert-butyl 3-(3-(4-chloro-3,5-dimethylphenoxy)propyl)-1-(2-(piperazin-1-yl)ethyl)-7-(1,3,5-trimethyl-1H-pyrazol-4-yl)-1H-indole-2-carboxylate (40.0 mg, 0.063 mmol) and 2-chloro-N-((2-(2,6-dioxopiperidin-3-yl)-1-oxoisoindolin-5-yl)methyl)acetamide (24.3 mg, 0.069 mmol) were dissolved in DMF (1.3 mL). Subsequently, KI (10.5 mg, 0.063 mmol) and DIPEA (0.033 mL, 0.189 mmol) were added and the mixture was stirring in 70° C. for 24 h. DMF was evaporated and the resultant residue was partitioned between EtOAc and H₂O. The organic layer was further washed with brine, dried over Na₂SO₄, filtered and evaporated. Desired product was purified using flash chromatography (SiO₂, 10% MeOH in DCM). Tert-butyl 3-(3-(4-chloro-3,5-dimethylphenoxy)propyl)-1-(2-(4-(2-(((2-(2,6-dioxopiperidin-3-yl)-1-oxoisoindolin-5-yl)methyl)amino)-2-oxoethyl)piperazin-1-yl)ethyl)-7-(1,3,5-trimethyl-1H-pyrazol-4-yl)-1H-indole-2-carboxylate (39.0 mg, 0.036 mmol, 57%) was obtained as orange oil.

LCMS (ESI+): m/z 948.1 [M+H]⁺

### Step E

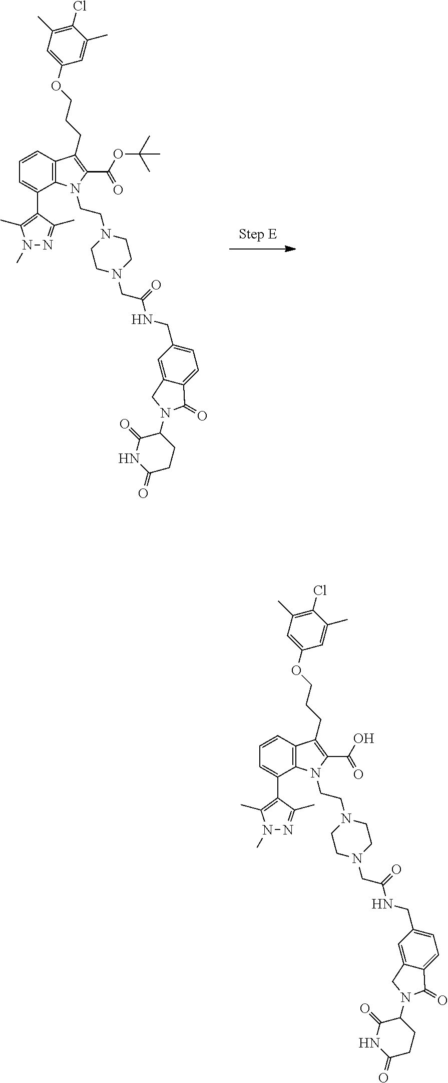 [SMILES: Cc1cc(OCCCc2c(C(=O)OC(C)(C)C)n(CCN3CCN(CC(=O)NCc4ccc5c(c4)CN(C4CCC(=O)NC4=O)C5=O)CC3)c3c(-c4c(C)nn(C)c4C)cccc23)cc(C)c1Cl ; Cc1cc(OCCCc2c(C(=O)O)n(CCN3CCN(CC(=O)NCc4ccc5c(c4)CN(C4CCC(=O)NC4=O)C5=O)CC3)c3c(-c4c(C)nn(C)c4C)cccc23)cc(C)c1Cl]

Tert-butyl 3-(3-(4-chloro-3,5-dimethylphenoxy)propyl)-1-(2-(4-(2-(((2-(2,6-dioxopiperidin-3-yl)-1-oxoisoindolin-5-yl)methyl)amino)-2-oxoethyl)piperazin-1-yl)ethyl)-7-(1,3,5-trimethyl-1H-pyrazol-4-yl)-1H-indole-2-carboxylate (40.0 mg, 0.042 mmol) was dissolved in 4 M HCl in dioxane (0.073 mL, 2.111 mmol). Subsequently, the mixture was stirring at room temperature for overnight. The solvent was evaporated and the resultant residue was dissolved in DMSO and filtered. The compound was purified by preparative HPLC (H₂O:MeCN+0.1% FA). 3-(3-(4-chloro-3,5-dimethylphenoxy)propyl)-1-(2-(4-(2-(((2-(2,6-dioxopiperidin-3-yl)-1-oxoisoindolin-5-yl)methyl)amino)-2-oxoethyl)piperazin-1-yl)ethyl)-7-(1,3,5-trimethyl-1H-pyrazol-4-yl)-1H-indole-2-carboxylic acid (13.0 mg, 0.015 mmol, 36%) was obtained as yellowish solid.

LCMS (ESI+): m/z 891.0 [M+H]⁺

¹H NMR (500 MHz, DMSO) δ 10.97 (s, 1H), 8.28 (s, 1H), 7.70-7.61 (m, 2H), 7.44 (s, 1H), 7.38 (d, J=7.8 Hz, 1H), 7.10 (t, J=7.6 Hz, 1H), 6.92 (dd, J=7.0, 1.2 Hz, 1H), 6.73 (s, 2H), 5.10 (dd, J=13.3, 5.1 Hz, 1H), 4.47-4.18 (m, 6H), 3.98 (t, J=6.4 Hz, 2H), 3.73-3.66 (m, 3H), 3.14 (t, J=7.4 Hz, 2H), 2.96-2.85 (m, 2H), 2.62-2.57 (m, 1H), 2.53-2.51 (m, 1H), 2.44-2.27 (m, 5H), 2.26 (s, 6H), 2.13 (s, 5H), 2.06 (s, 3H), 2.06-1.96 (m, 4H), 1.92 (s, 3H).

### Example 35. 1-(2-(4-(2-((2-(2,6-dioxopiperidin-3-yl)-1,3-dioxoisoindolin-4-yl)oxy)acetyl)piperazin-1-yl)ethyl)-3-(3-(naphthalen-1-yloxy)propyl)-7-(1,3,5-trimethyl-1H-pyrazol-4-yl)-1H-indole-2-carboxylic acid (241)

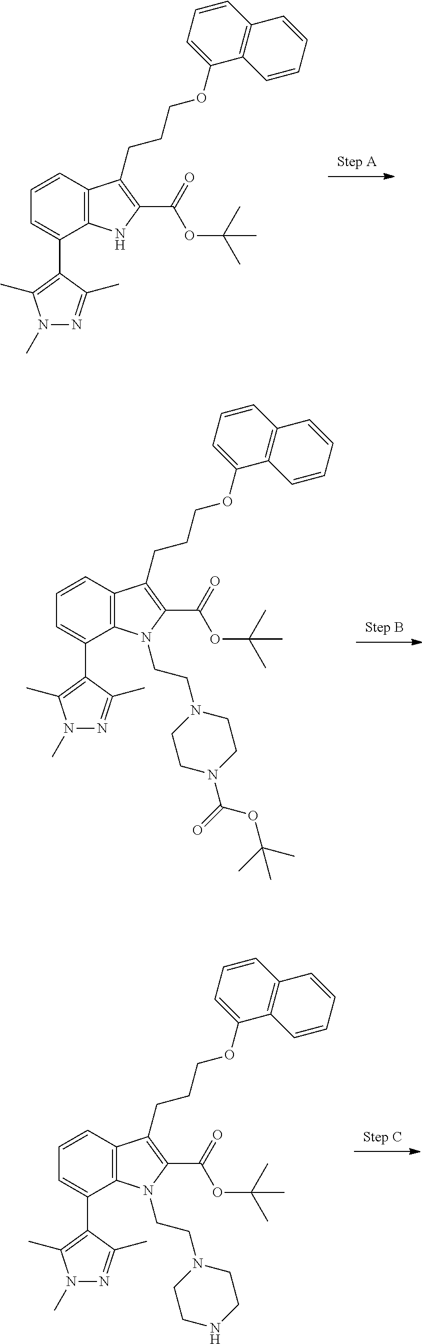 [SMILES: Cc1nn(C)c(C)c1-c1cccc2c(CCCOc3cccc4ccccc34)c(C(=O)OC(C)(C)C)[nH]c12 ; Cc1nn(C)c(C)c1-c1cccc2c(CCCOc3cccc4ccccc34)c(C(=O)OC(C)(C)C)n(CCN3CCN(C(=O)OC(C)(C)C)CC3)c12 ; Cc1nn(C)c(C)c1-c1cccc2c(CCCOc3cccc4ccccc34)c(C(=O)OC(C)(C)C)n(CCN3CCNCC3)c12]

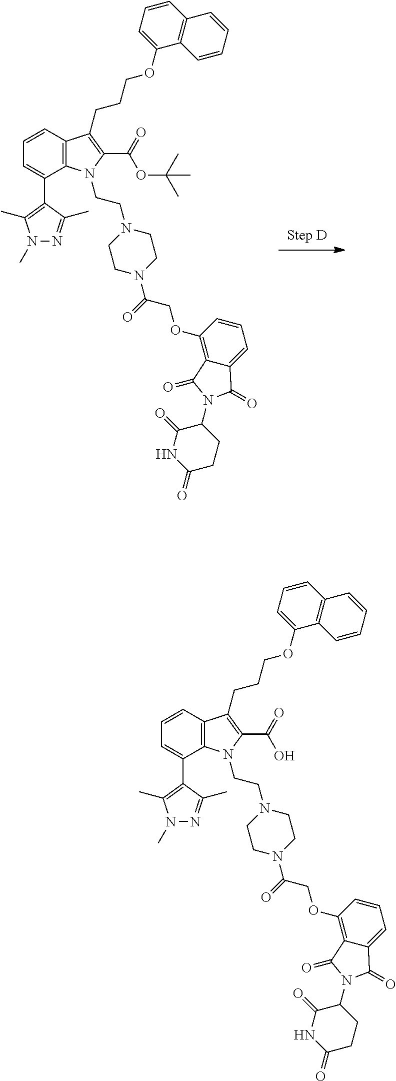 [SMILES: Cc1nn(C)c(C)c1-c1cccc2c(CCCOc3cccc4ccccc34)c(C(=O)OC(C)(C)C)n(CCN3CCN(C(=O)COc4cccc5c4C(=O)N(C4CCC(=O)NC4=O)C5=O)CC3)c12 ; Cc1nn(C)c(C)c1-c1cccc2c(CCCOc3cccc4ccccc34)c(C(=O)O)n(CCN3CCN(C(=O)COc4cccc5c4C(=O)N(C4CCC(=O)NC4=O)C5=O)CC3)c12]

### Step A

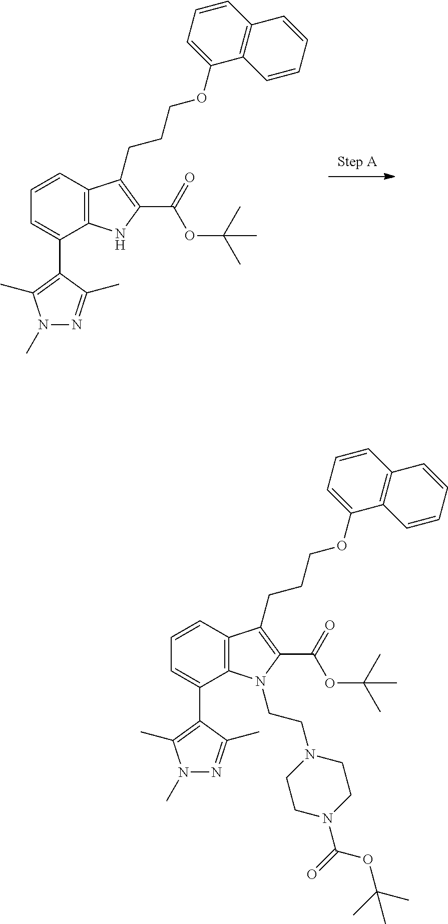 [SMILES: Cc1nn(C)c(C)c1-c1cccc2c(CCCOc3cccc4ccccc34)c(C(=O)OC(C)(C)C)[nH]c12 ; Cc1nn(C)c(C)c1-c1cccc2c(CCCOc3cccc4ccccc34)c(C(=O)OC(C)(C)C)n(CCN3CCN(C(=O)OC(C)(C)C)CC3)c12]

Tert-butyl 3-(3-(naphthalen-1-yloxy)propyl)-7-(1,3,5-trimethyl-1H-pyrazol-4-yl)-1H-indole-2-carboxylate (300.0 mg, 0.589 mmol) was dissolved in dry DMF (5.9 mL). Cs₂CO₃ (958.9 mg, 1.766 mmol), KI (97.7 mg, 0.589 mmol) and tert-butyl 4-(2-chloroethyl)piperazine-1-carboxylate (161.0 mg, 0.648 mmol) were added and the reaction was stirred in 70° C. After 20 h, DMF was evaporated, and the residues were dissolved in EtOAc. Organic layer was washed 3× with water and 3× with brine. The organic phase was collected and combined aqueous layer was 2× washed with EtOAc. Merged organic layers were once again washed with water, dried over anhydrous Na₂SO₄, filtered and the solvent was evaporated. The reaction product was dried under reduced pressure, to afford tert-butyl 1-(2-(4-(tert-butoxycarbonyl)piperazin-1-yl)ethyl)-3-(3-(naphthalen-1-yloxy)propyl)-7-(1,3,5-trimethyl-1H-pyrazol-4-yl)-1H-indole-2-carboxylate (404.0 mg, crude) as yellow oil.

LCMS (ESI+): m/z 722.0 [M+H]⁺

### Step B

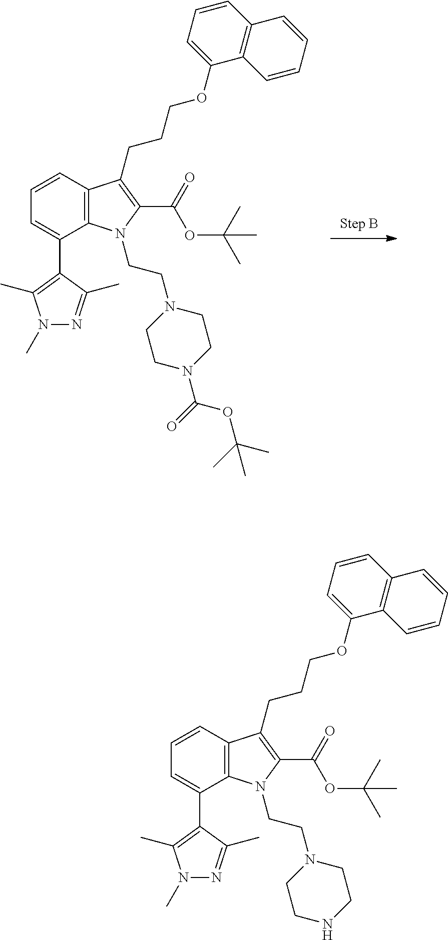 [SMILES: Cc1nn(C)c(C)c1-c1cccc2c(CCCOc3cccc4ccccc34)c(C(=O)OC(C)(C)C)n(CCN3CCN(C(=O)OC(C)(C)C)CC3)c12 ; Cc1nn(C)c(C)c1-c1cccc2c(CCCOc3cccc4ccccc34)c(C(=O)OC(C)(C)C)n(CCN3CCNCC3)c12]

Tert-butyl 1-(2-(4-(tert-butoxycarbonyl)piperazin-1-yl)ethyl)-3-(3-(naphthalen-1-yloxy)propyl)-7-(1,3,5-trimethyl-1H-pyrazol-4-yl)-1H-indole-2-carboxylate (404.0 mg, crude) was dissolved in dry THE (11.2 mL) under an inert gas atmosphere, cooled down to 0° C. and 4 M solution of HCl in dioxane (7.0 mL, 27.980 mmol) was added. After 9 h full conversion of the starting material was observed. The reaction was quenched by the addition of cold 1 M NaOH aqueous solution to neutral pH. Reaction product was extracted several times with EtOAc. The combined organics were dried over anhydrous Na₂SO₄ and concentrated in vacuo. Desired product was purified using flash chromatography (SiO₂, 20% MeOH in DCM). Tert-butyl 3-(3-(naphthalen-1-yloxy)propyl)-1-(2-(piperazin-1-yl)ethyl)-7-(1,3,5-trimethyl-1H-pyrazol-4-yl)-1H-indole-2-carboxylate (293.0 mg, 0.451 mmol, 77% yield) was obtained as orange oil.

LCMS (ESI+): m/z 622.8 [M+H]⁺

### Step C

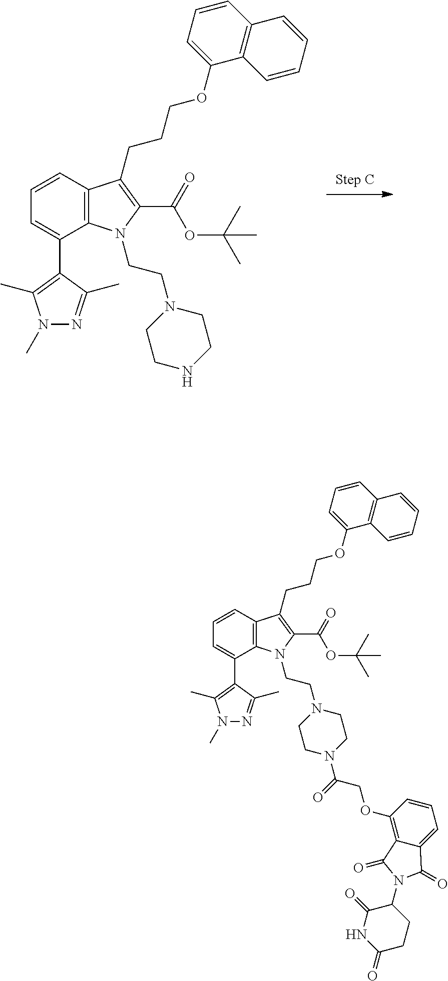 [SMILES: Cc1nn(C)c(C)c1-c1cccc2c(CCCOc3cccc4ccccc34)c(C(=O)OC(C)(C)C)n(CCN3CCNCC3)c12 ; Cc1nn(C)c(C)c1-c1cccc2c(CCCOc3cccc4ccccc34)c(C(=O)OC(C)(C)C)n(CCN3CCN(C(=O)COc4cccc5c4C(=O)N(C4CCC(=O)NC4=O)C5=O)CC3)c12]

To a solution of 2-((2-(2,6-dioxopiperidin-3-yl)-1,3-dioxoisoindolin-4-yl)oxy)acetic acid (19.2 mg, 0.058 mmol) in dry DMF (1.9 mL), DIPEA (0.025 mL, 0.145 mmol) and HATU (27.5 mg, 0.072 mmol) were added and the mixture was allowed to stir under argon atmosphere for 15 min. Next, tert-butyl 3-(3-(naphthalen-1-yloxy)propyl)-1-(2-(piperazin-1-yl)ethyl)-7-(1,3,5-trimethyl-1H-pyrazol-4-yl)-1H-indole-2-carboxylate (30.0 mg, 0.048 mmol) was added and the solution was stirred for 2 h at room temperature. DMF was evaporated, the reaction mixture diluted with EtOAc, washed successively with cold water (3 times) and brine. Organic layer was dried over Na₂SO₄ and evaporated under reduced pressure to afford tert-butyl 1-(2-(4-(2-((2-(2,6-dioxopiperidin-3-yl)-1,3-dioxoisoindolin-4-yl)oxy)acetyl)piperazin-1-yl)ethyl)-3-(3-(naphthalen-1-yloxy)propyl)-7-(1,3,5-trimethyl-1H-pyrazol-4-yl)-1H-indole-2-carboxylate (36.6 mg, crude) as a black solid which was used in next step without further purification.

LCMS (ESI+): m/z 936.87 [M+H]⁺

### Step D

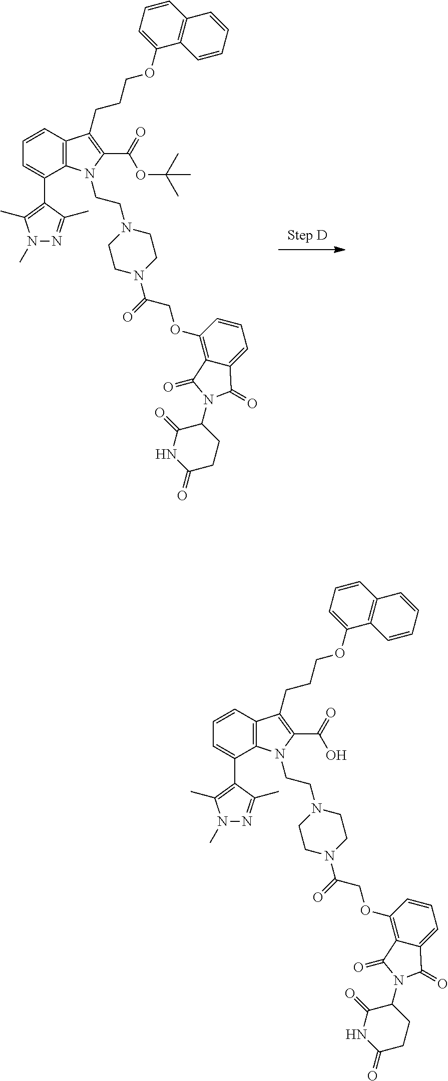 [SMILES: Cc1nn(C)c(C)c1-c1cccc2c(CCCOc3cccc4ccccc34)c(C(=O)OC(C)(C)C)n(CCN3CCN(C(=O)COc4cccc5c4C(=O)N(C4CCC(=O)NC4=O)C5=O)CC3)c12 ; Cc1nn(C)c(C)c1-c1cccc2c(CCCOc3cccc4ccccc34)c(C(=O)O)n(CCN3CCN(C(=O)COc4cccc5c4C(=O)N(C4CCC(=O)NC4=O)C5=O)CC3)c12]

Tert-butyl 1-(2-(4-(2-((2-(2,6-dioxopiperidin-3-yl)-1,3-dioxoisoindolin-4-yl)oxy)acetyl)piperazin-1-yl)ethyl)-3-(3-(naphthalen-1-yloxy)propyl)-7-(1,3,5-trimethyl-1H-pyrazol-4-yl)-1H-indole-2-carboxylate (36.6 mg, crude) was dissolved in dry DCM (0.18 mL) under argon atmosphere. TFA (0.18 mL, 0.782 mmol) was added and the reaction was stirred at room temperature in a sealed vial for 18 h. DCM and TFA were evaporated under reduced pressure. The residues were dissolved in DMSO and purified by preparative HPLC (H₂O:MeCN+0.1% FA) to give a corresponding 1-(2-(4-(2-((2-(2,6-dioxopiperidin-3-yl)-1,3-dioxoisoindolin-4-yl)oxy)acetyl)piperazin-1-yl)ethyl)-3-(3-(naphthalen-1-yloxy)propyl)-7-(1,3,5-trimethyl-1H-pyrazol-4-yl)-1H-indole-2-carboxylic acid (12.7 mg, 0.014 mmol, 29% yield over two steps) as white powder.

LCMS (ESI+): m/z 881.1 [M+H]⁺

¹H NMR (500 MHz, DMSO) δ 13.27 (s, 1H), 11.11 (s, 1H), 8.30-8.22 (m, 1H), 7.92-7.86 (m, 1H), 7.83-7.69 (m, 2H), 7.57-7.50 (m, 2H), 7.47 (t, J=6.8 Hz, 2H), 7.41 (t, J=7.9 Hz, 1H), 7.32 (d, J=7.2 Hz, 1H), 7.15-7.09 (m, 1H), 7.00-6.95 (m, 1H), 6.93 (d, J=7.5 Hz, 1H), 5.29-5.01 (m, 3H), 4.61-4.27 (m, 2H), 4.25 (t, J=6.0 Hz, 2H), 3.77 (s, 3H), 3.42-3.36 (m, 4H), 3.30-3.27 (m, 2H), 2.96-2.84 (m, 1H), 2.64-2.57 (m, 2H), 2.56-2.54 (m, 1H), 2.25 (d, J=6.8 Hz, 2H), 2.11 (s, 3H), 2.10-1.99 (m, 5H), 1.96 (s, 3H), 1.96-1.88 (m, 1H)

### Example 36. 3-(3-(4-chloro-3,5-dimethylphenoxy)propyl)-N-((2-(5-((2-(2,6-dioxopiperidin-3-yl)-1,3-dioxoisoindolin-4-yl)amino)pentanamido)ethyl)sulfonyl)-7-(1,3,5-trimethyl-1H-pyrazol-4-yl)-1H-indole-2-carboxamide (242)

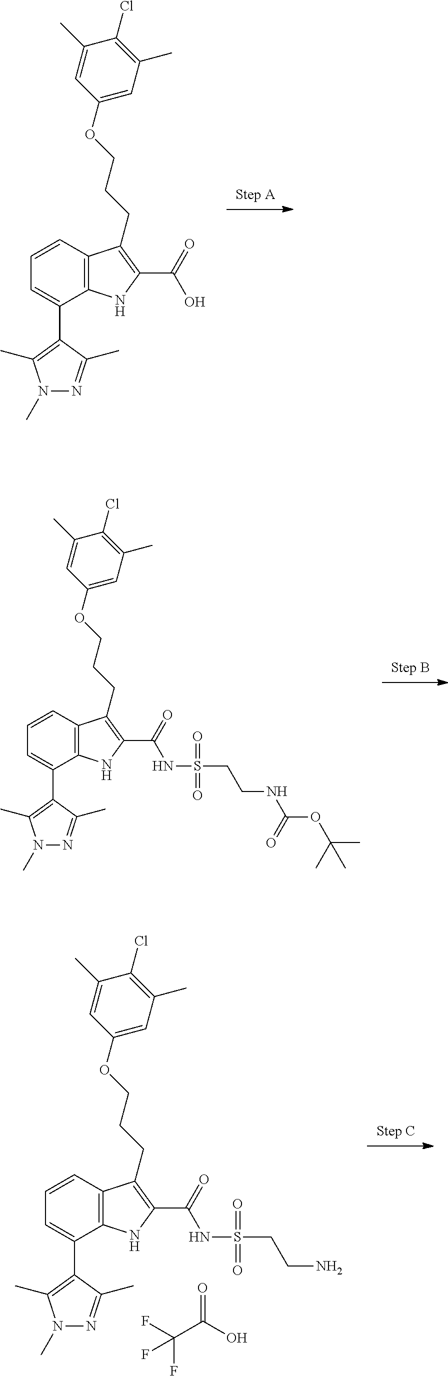

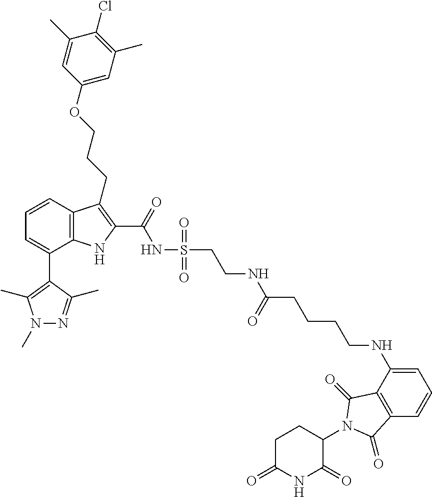 [SMILES: Cc1cc(OCCCc2c(C(=O)NS(=O)(=O)CCNC(=O)CCCCNc3cccc4c3C(=O)N(C3CCC(=O)NC3=O)C4=O)[nH]c3c(-c4c(C)nn(C)c4C)cccc23)cc(C)c1Cl]

### Step A

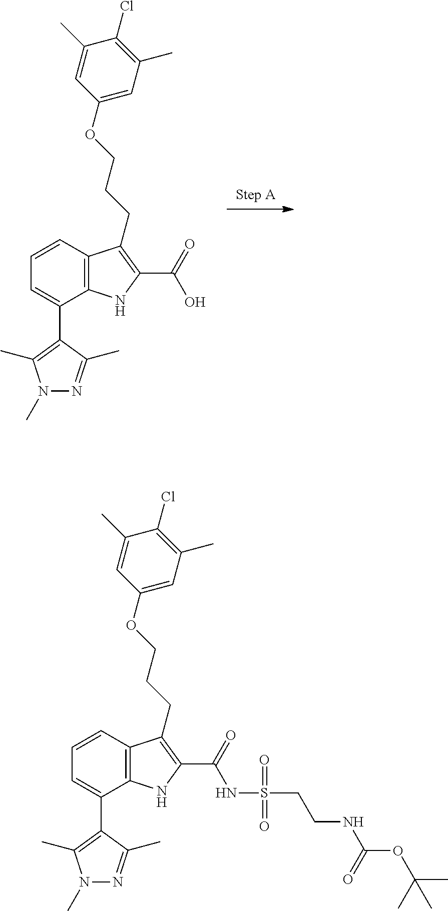 [SMILES: Cc1cc(OCCCc2c(C(=O)O)[nH]c3c(-c4c(C)nn(C)c4C)cccc23)cc(C)c1Cl ; Cc1cc(OCCCc2c(C(=O)NS(=O)(=O)CCNC(=O)OC(C)(C)C)[nH]c3c(-c4c(C)nn(C)c4C)cccc23)cc(C)c1Cl]

3-(3-(4-chloro-3,5-dimethylphenoxy)propyl)-7-(1,3,5-trimethyl-1H-pyrazol-4-yl)-1H-indole-2-carboxylic acid (6.6 g, 14.2 mmol), Et₃N (5 mL, 28.1 mmol), DMAP (0.86 g, 7.1 mmol) and HATU (8.6 g, 22.6 mmol) were dissolved in DMF (100 mL) and reaction mixture was stirred at room temperature for 15 min. Followed by tert-butyl (2-sulfamoylethyl)carbamate (4.75 g, 21.2 mmol) was added in one portion and the reaction mixture was stirred at 50° C. for 24 h. The mixture was poured into water and the aqueous solution was extracted with DCM. The combined organic extracts were washed with H₂O and dried over Na₂SO₄. The solvent was removed under vacuum and the residue was purified by flash chromatography to afford tert-butyl (2-(N-(3-(3-(4-chloro-3,5-dimethylphenoxy)propyl)-7-(1,3,5-trimethyl-1H-pyrazol-4-yl)-1H-indole-2-carbonyl)sulfamoyl)ethyl)carbamate (3.4 g, 5.06 mmol, 36%).

### Step B

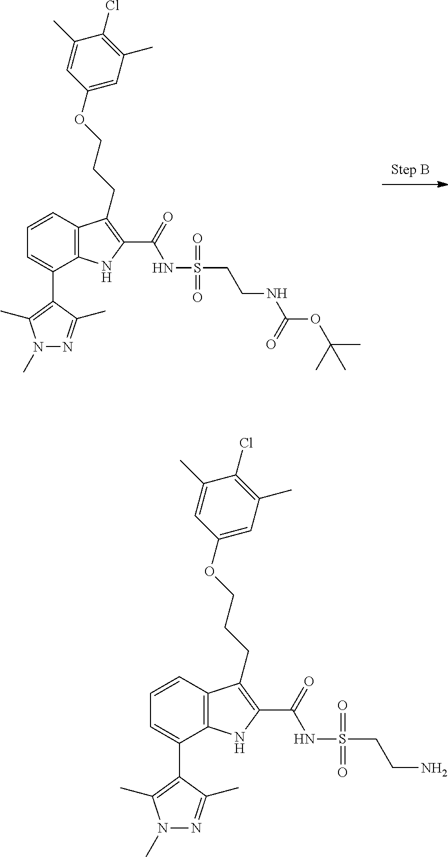

Tert-butyl (2-(N-(3-(3-(4-chloro-3,5-dimethylphenoxy)propyl)-7-(1,3,5-trimethyl-1H-pyrazol-4-yl)-1H-indole-2-carbonyl)sulfamoyl)ethyl)carbamate (3.4 g, 5.06 mmol) was dissolved in DCM (50 mL) and TFA (10 mL) was added dropwise to the mixture. The reaction was stirred at room temperature for 24 h and then evaporated under reduced pressure to give crude N-((2-aminoethyl)sulfonyl)-3-(3-(4-chloro-3,5-dimethylphenoxy)propyl)-7-(1,3,5-trimethyl-1H-pyrazol-4-yl)-1H-indole-2-carboxamide trifluoroacetate (4.10 g of crude), which was used into the next step without further purification.

### Step C

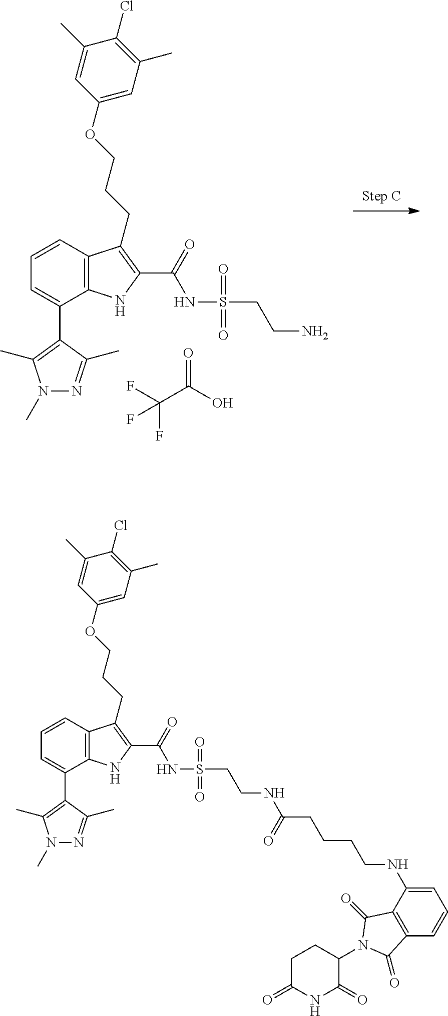

5-((2-(2,6-dioxopiperidin-3-yl)-1,3-dioxoisoindolin-4-yl)amino)pentanoic acid (0.45 g, 1.21 mmol), DIPEA (0.61 mL, 3.50 mmol), and HATU (0.50 g, 1.30 mmol) were dissolved in DMF (6 mL) and the reaction mixture were stirred at room temperature for 15 min. Followed by N-((2-aminoethyl)sulfonyl)-3-(3-(4-chloro-3,5-dimethylphenoxy)propyl)-7-(1,3,5-trimethyl-1H-pyrazol-4-yl)-1H-indole-2-carboxamide trifluoroacetate (0.60 g, 0.87 mmol) in DMF (5 mL) was added in one portion and the reaction mixture were stirred at 50° C. for 24 h. The mixture was poured into water and the aqueous solution was extracted with DCM. The combined organic extracts were washed with H₂O and dried over Na₂SO₄. The solvent was removed under vacuum and the residue was purified by preparative HPLC (H₂O:MeCN+0.1% FA) to afford 3-(3-(4-chloro-3,5-dimethylphenoxy)propyl)-N-((2-(5-((2-(2,6-dioxopiperidin-3-yl)-1,3-dioxoisoindolin-4-yl)amino)pentanamido)ethyl)sulfonyl)-7-(1,3,5-trimethyl-1H-pyrazol-4-yl)-1H-indole-2-carboxamide (64 mg, 0.069 mmol, 8% over two steps) as yellow solid.

LCMS (ESI+): m/z 927.7 [M+H]⁺

¹H NMR (400 MHz, CD₃OD) δ 7.64-7.55 (m, 1H), 7.52-7.42 (m, 1H), 7.05 (t, J=7.5 Hz, 1H), 7.02-6.96 (m, 2H), 6.87 (d, J=8.6 Hz, 1H), 6.71-6.60 (m, 2H), 5.04 (dd, J=12.5, 5.4 Hz, 1H), 3.98 (t, J=6.4 Hz, 2H), 3.82 (s, 3H), 3.63 (t, J=6.3 Hz, 2H), 3.46 (t, J=6.3 Hz, 2H), 3.39-3.34 (m, 2H), 3.08 (t, J=6.9 Hz, 1H), 2.86-2.70 (m, 2H), 2.68-2.64 (m, 4H), 2.29 (s, 6H), 2.20-2.03 (m, 11H), 1.58-1.39 (m, 4H).

### Example 37. 3-(3-(4-chloro-3,5-dimethylphenoxypropyl)-N-((2-(4-(2-((2-(2,6-dioxopiperidin-3-yl)-1,3-dioxoisoindolin-4-yl)amino)ethoxy)butanamido)ethyl)sulfonyl)-7-(1,3,5-trimethyl-1H-pyrazol-4-yl)-1H-indole-2-carboxamide (243)

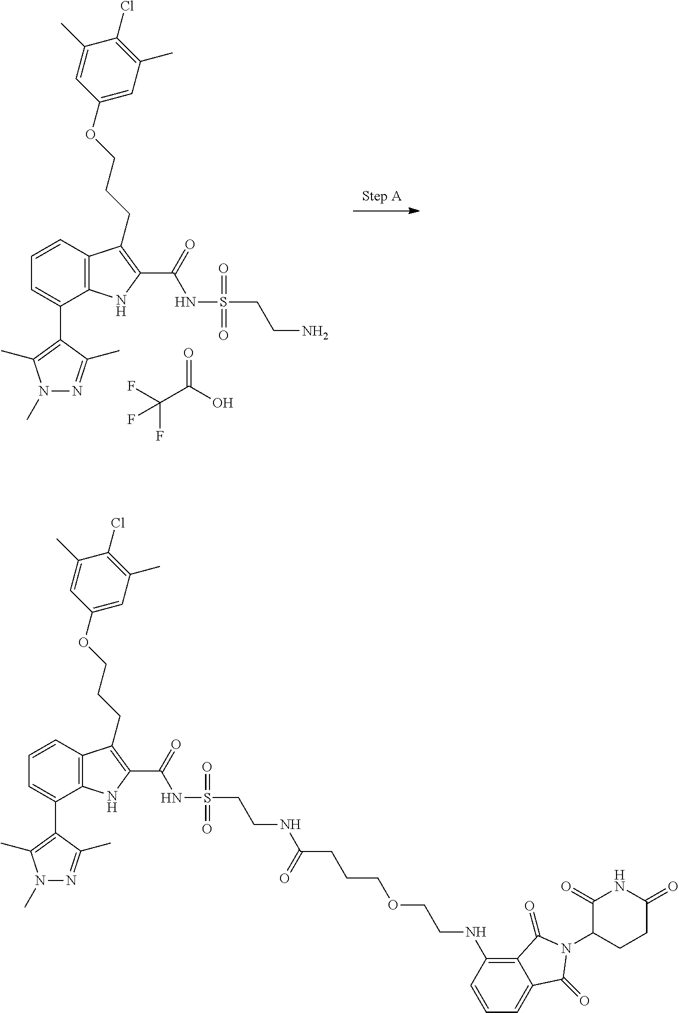

### Step A

4-(2-((2-(2,6-dioxopiperidin-3-yl)-1,3-dioxoisoindolin-4-yl)amino)ethoxy)butanoic acid (0.49 g, 1.21 mmol), DIPEA (0.61 mL, 3.50 mmol) and HATU (0.50 g, 1.30 mmol) were dissolved in DMF (6 mL) and the reaction mixture were stirred at room temperature for 15 min. Followed N-((2-aminoethyl)sulfonyl)-3-(3-(4-chloro-3,5-dimethylphenoxy)propyl)-7-(1,3,5-trimethyl-1H-pyrazol-4-yl)-1H-indole-2-carboxamide trifluoroacetate (0.60 g, 0.87 mmol) in DMF (5 mL) was added in one portion and the reaction mixture were stirred at 50° C. for 24 h. The mixture was poured into water and the aqueous solution was extracted with DCM. The combined organic extracts were washed with H₂O and dried over Na₂SO₄. The solvent was removed under vacuum and the residue was purified by preparative HPLC to afford 3-(3-(4-chloro-3,5-dimethylphenoxy)propyl)-N-((2-(4-(2-((2-(2,6-dioxopiperidin-3-yl)-1,3-dioxoisoindolin-4-yl)amino)ethoxy)butanamido)ethyl)sulfonyl)-7-(1,3,5-trimethyl-1H-pyrazol-4-yl)-1H-indole-2-carboxamide (0.06 g, 0.063 mmol, 7%) as a yellow solid.

LCMS (ESI+): m/z 957.2 [M+H]⁺

¹H NMR (400 MHz, CDCl₃) δ 7.67 (d, J=8.0 Hz, 1H), 7.49 (t, J=7.8 Hz, 1H), 7.20-7.03 (m, 3H), 6.94-6.80 (m, 2H), 6.66 (s, 2H), 6.56-6.47 (m, 1H), 5.06-4.93 (m, 1H), 3.96 (t, J=6.0 Hz, 2H), 3.84-3.66 (m, 7H), 3.66-3.54 (m, 2H), 3.49-3.27 (m, 6H), 2.92-2.67 (m, 3H), 2.38-2.25 (m, 8H), 2.24-2.10 (m, 6H), 2.05 (s, 3H), 1.83 (s, 2H).

### Example 38. 3-(3-(4-chloro-3,5-dimethylphenoxy)propyl)-N-((2-(3-(2-(2-((2-(2,6-dioxopiperidin-3-yl)-1,3-dioxoisoindolin-4-yl)amino)ethoxy)ethoxy)propanamido)ethyl)sulfonyl)-7-(1,3,5-trimethyl-1H-pyrazol-4-yl)-1H-indole-2-carboxamide (244)

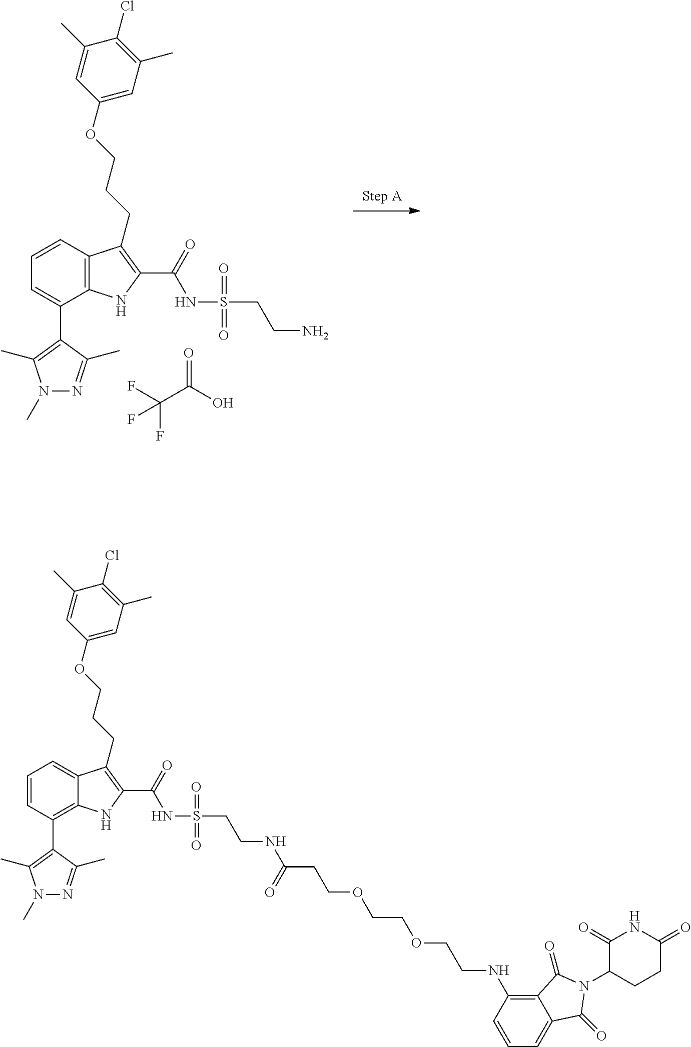

### Step A

3-(2-(2-((2-(2,6-dioxopiperidin-3-yl)-1,3-dioxoisoindolin-4-yl)amino)ethoxy)ethoxy)propanoic acid (0.53 g, 1.22 mmol), DIPEA (0.61 mL, 3.50 mmol), and HATU (0.50 g, 1.30 mmol) were dissolved in DMF (6 mL) and the reaction mixture were stirred at room temperature for 15 min. Followed by N-((2-aminoethyl)sulfonyl)-3-(3-(4-chloro-3,5-dimethylphenoxy)propyl)-7-(1,3,5-trimethyl-1H-pyrazol-4-yl)-1H-indole-2-carboxamide trifluoroacetate (0.60 g, 0.87 mmol) in DMF (5 mL) was added in one portion and the reaction mixture were stirred at 50° C. for 24 h. The mixture was poured into water and the aqueous solution was extracted with DCM. The combined organic extracts were washed with H₂O and dried over Na₂SO₄. The solvent was removed under vacuum and the residue was purified by preparative HPLC (H₂O:MeCN+0.1% FA) to afford 3-(3-(4-chloro-3,5-dimethylphenoxy)propyl)-N-((2-(3-(2-(2-((2-(2,6-dioxopiperidin-3-yl)-1,3-dioxoisoindolin-4-yl)amino)ethoxy)ethoxy)propanamido)ethyl)sulfonyl)-7-(1,3,5-trimethyl-1H-pyrazol-4-yl)-1H-indole-2-carboxamide (92 mg, 0.093 mmol, 11%) as a yellow solid.

LCMS (ESI+): m/z 987.7 [M+H]⁺

¹H NMR (600 MHz, DMSO) δ 11.70 (s, 1H), 11.09 (s, 1H), 10.78 (s, 1H), 8.10-8.00 (m, 1H), 7.64 (d, J=8.0 Hz, 1H), 7.55 (dd, J=8.6, 7.0 Hz, 1H), 7.16-7.05 (m, 3H), 7.02 (d, J=7.0 Hz, 1H), 6.76 (s, 2H), 6.57 (t, J=5.8 Hz, 1H), 5.04 (dd, J=12.9, 5.5 Hz, 1H), 3.97 (t, J=6.4 Hz, 2H), 3.77 (s, 3H), 3.71-3.62 (m, 2H), 3.56 (t, J=5.5 Hz, 2H), 3.49-3.40 (m, 8H), 3.38-3.34 (m, 2H), 3.23 (t, J=8.5, 6.5 Hz, 2H), 2.87 (ddd, J=17.0, 13.8, 5.4 Hz, 1H), 2.61-2.51 (m, 2H), 2.26 (s, 6H), 2.20 (t, J=6.6 Hz, 2H), 2.09 (s, 3H), 2.06-1.98 (m, 6H).

### Example 39. 7-(5-((4-(4-(N,N-dimethylsulfamoyl)piperazin-1-yl)phenoxy)methyl)-1,3-dimethyl-1H-pyrazol-4-yl)-1-(2-(4-((2-(2,6-dioxopiperidin-3-yl)-1,3-dioxoisoindolin-4-yl)glycyl)piperazin-1-yl)ethyl)-3-(3-(naphthalen-1-yloxy)propyl)-1H-indole-2-carboxylic acid (245)

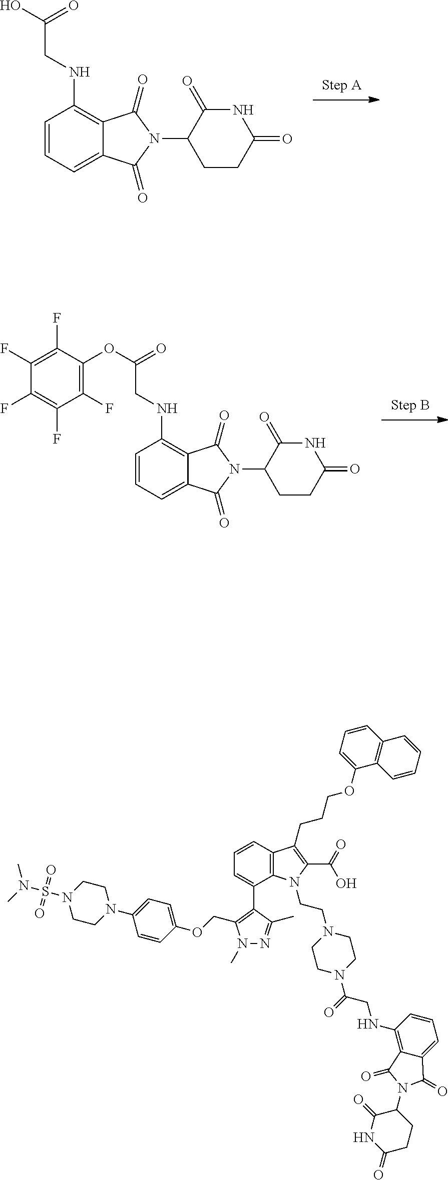 [SMILES: O=C(O)CNc1cccc2c1C(=O)N(C1CCC(=O)NC1=O)C2=O ; O=C1CCC(N2C(=O)c3cccc(NCC(=O)Oc4c(F)c(F)c(F)c(F)c4F)c3C2=O)C(=O)N1 ; Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(C(=O)CNc4cccc5c4C(=O)N(C4CCC(=O)NC4=O)C5=O)CC3)c12]

### Step A

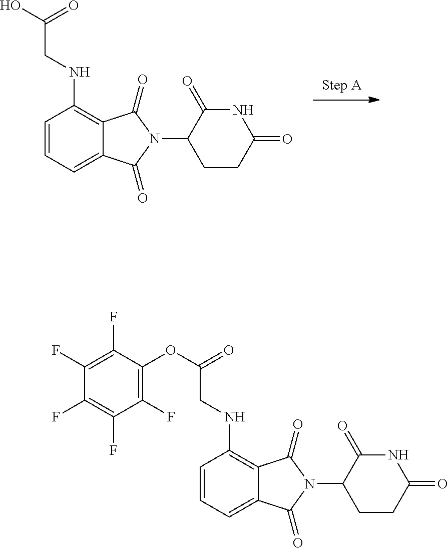 [SMILES: O=C(O)CNc1cccc2c1C(=O)N(C1CCC(=O)NC1=O)C2=O ; O=C1CCC(N2C(=O)c3cccc(NCC(=O)Oc4c(F)c(F)c(F)c(F)c4F)c3C2=O)C(=O)N1]

To a solution of (2-(2,6-dioxopiperidin-3-yl)-1,3-dioxoisoindolin-4-yl)glycine (200 mg, 0.60 mmol) in DMF (2 ml), DIPEA (1 ml, 6 mmol) was added and the reaction mixture was cooled down to 0° C. Pentafluorophenyl trifluoroacetate (843 mg, 3.0 mmol) was added and the mixture was allowed to stir at ambient temperature for 2 h under nitrogen. After complete consumption of the starting material (monitored by LCMS) the volatiles were evaporated under reduced pressure to obtain 200 mg of crude perfluorophenyl (2-(2,6-dioxopiperidin-3-yl)-1,3-dioxoisoindolin-4-yl)glycinate as brown sticky solid which was used for the next step without further purification.

### Step B

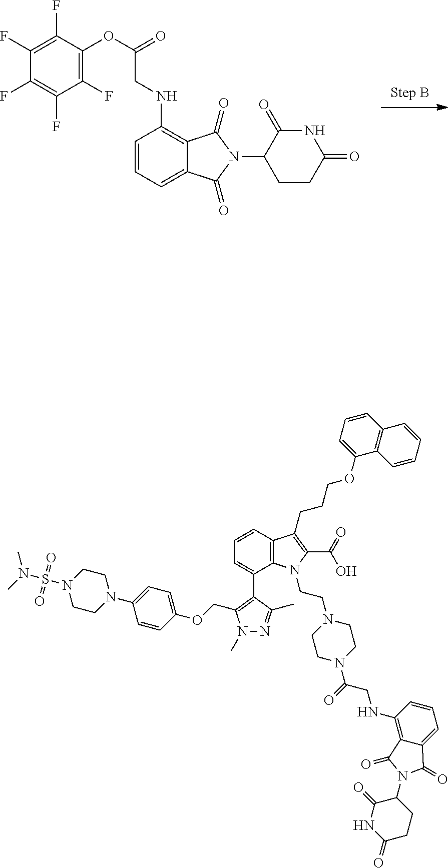 [SMILES: O=C1CCC(N2C(=O)c3cccc(NCC(=O)Oc4c(F)c(F)c(F)c(F)c4F)c3C2=O)C(=O)N1 ; Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(C(=O)CNc4cccc5c4C(=O)N(C4CCC(=O)NC4=O)C5=O)CC3)c12]

To a solution of 200 mg crude perfluorophenyl (2-(2,6-dioxopiperidin-3-yl)-1,3-dioxoisoindolin-4-yl)glycinate in DMF (2 mL), DIPEA (85 μL, 0.7 mmol) was added, followed by a solution of 7-(5-((4-(4-(N,N-dimethylsulfamoyl)piperazin-1-yl)phenoxy)methyl)-1,3-dimethyl-1H-pyrazol-4-yl)-3-(3-(naphthalen-1-yloxy)propyl)-1-(2-(piperazin-1-yl)ethyl)-1H-indole-2-carboxylic acid (120 mg, 0.14 mmol) in DMF (1 ml) at 0° C. under nitrogen. The reaction mixture was allowed to stir at ambient temperature for 16 h. After complete consumption of the starting material (monitored by LCMS), the reaction mixture was concentrated in vacuo and purified by preparative HPLC to get 7-(5-((4-(4-(N,N-dimethylsulfamoyl)piperazin-1-yl)phenoxy)methyl)-1,3-dimethyl-1H-pyrazol-4-yl)-1-(2-(4-((2-(2,6-dioxopiperidin-3-yl)-1,3-dioxoisoindolin-4-yl)glycyl)piperazin-1-yl)ethyl)-3-(3-(naphthalen-1-yloxy)propyl)-1H-indole-2-carboxylic acid (20 mg, 0.017 mmol, 12% over two steps) as white solid.

LCMS (ESI+): m/z 1162.8 [M+H]⁺

¹H NMR (400 MHz, DMSO) δ 13.25 (s, 1H), 11.09 (s, 1H), 8.25-8.18 (m, 1H), 7.88-7.81 (m, 1H), 7.73 (dd, J=8.0, 1.2 Hz, 1H), 7.63-7.54 (m, 1H), 7.54-7.46 (m, 2H), 7.44 (d, J=8.2 Hz, 1H), 7.36 (t, J=7.9 Hz, 1H), 7.10-7.02 (m, 3H), 6.93-6.84 (m, 2H), 6.79 (s, 4H), 5.06 (dd, J=12.9, 5.4 Hz, 1H), 4.93-4.78 (m, 2H), 4.66-4.48 (m, 1H), 4.20 (t, J=6.2 Hz, 3H), 4.08 (s, 2H), 3.88 (s, 3H), 3.47-3.32 (m, 6H), 3.24-3.16 (m, 4H), 2.99-2.91 (m, 4H), 2.91-2.83 (m, 1H), 2.77 (s, 6H), 2.63-2.53 (m, 2H), 2.27-2.16 (m, 2H), 2.15-2.00 (m, 7H), 1.97 (s, 3H).

### Example 40. 7-(5-((4-(4-(N,N-dimethylsulfamoyl)piperazin-1-yl)phenoxy)methyl)-1,3-dimethyl-1H-pyrazol-4-yl)-1-(2-(4-(3-(2-(2-((2-(2,6-dioxopiperidin-3-yl)-1,3-dioxoisoindolin-4-yl)amino)ethoxy)ethoxy)propyl)piperazin-1-yl)ethyl)-3-(3-(naphthalen-1-yloxy)propyl)-1H-indole-2-carboxylic acid trifluoroacetate (247)

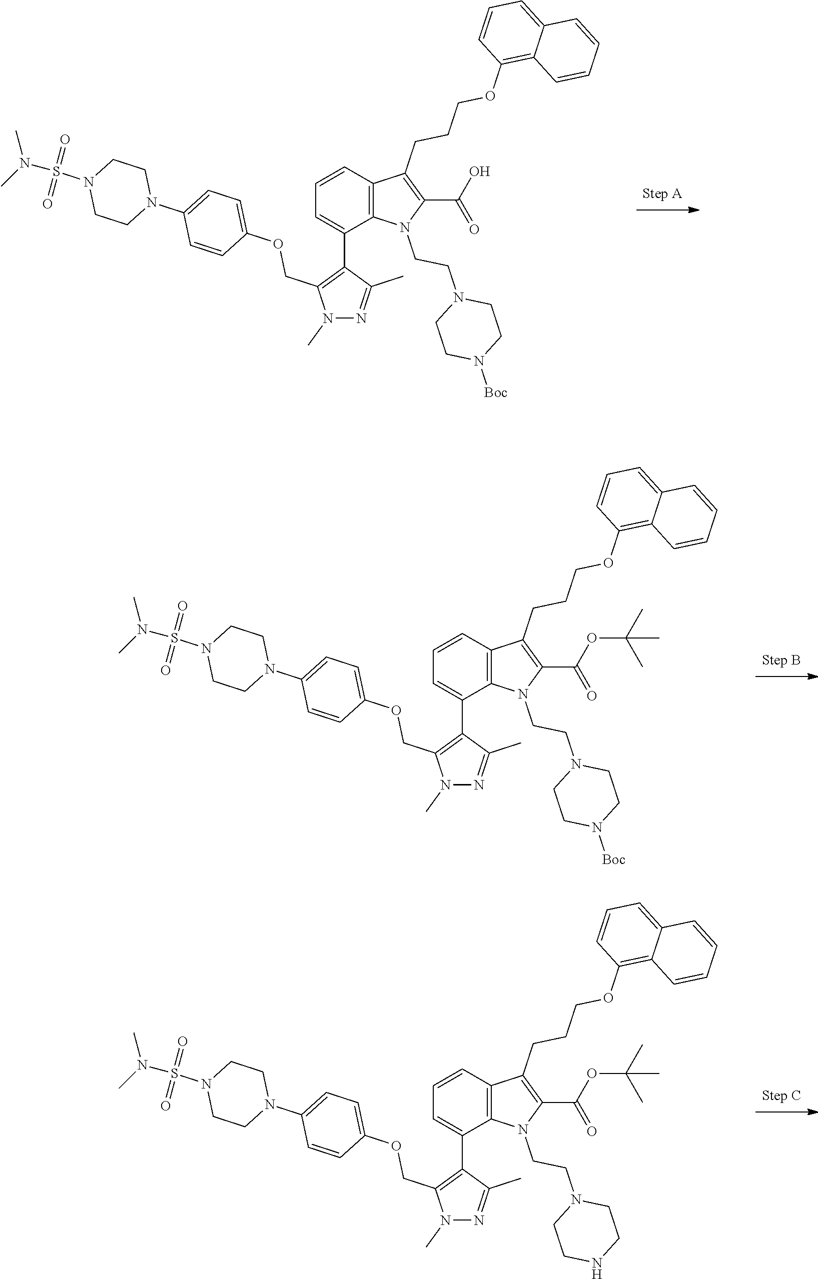 [SMILES: Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(C(=O)OC(C)(C)C)CC3)c12 ; Cc1nn(C)c(COc2ccc(N3CCN(S(=O)(=O)N(C)C)CC3)cc2)c1-c1cccc2c(CCCOc3cccc4ccccc34)c(C(=O)OC(C)(C)C)n(CCN3CCN(C(=O)OC(C)(C)C)CC3)c12 ; Cc1nn(C)c(COc2ccc(N3CCN(S(=O)(=O)N(C)C)CC3)cc2)c1-c1cccc2c(CCCOc3cccc4ccccc34)c(C(=O)OC(C)(C)C)n(CCN3CCNCC3)c12]

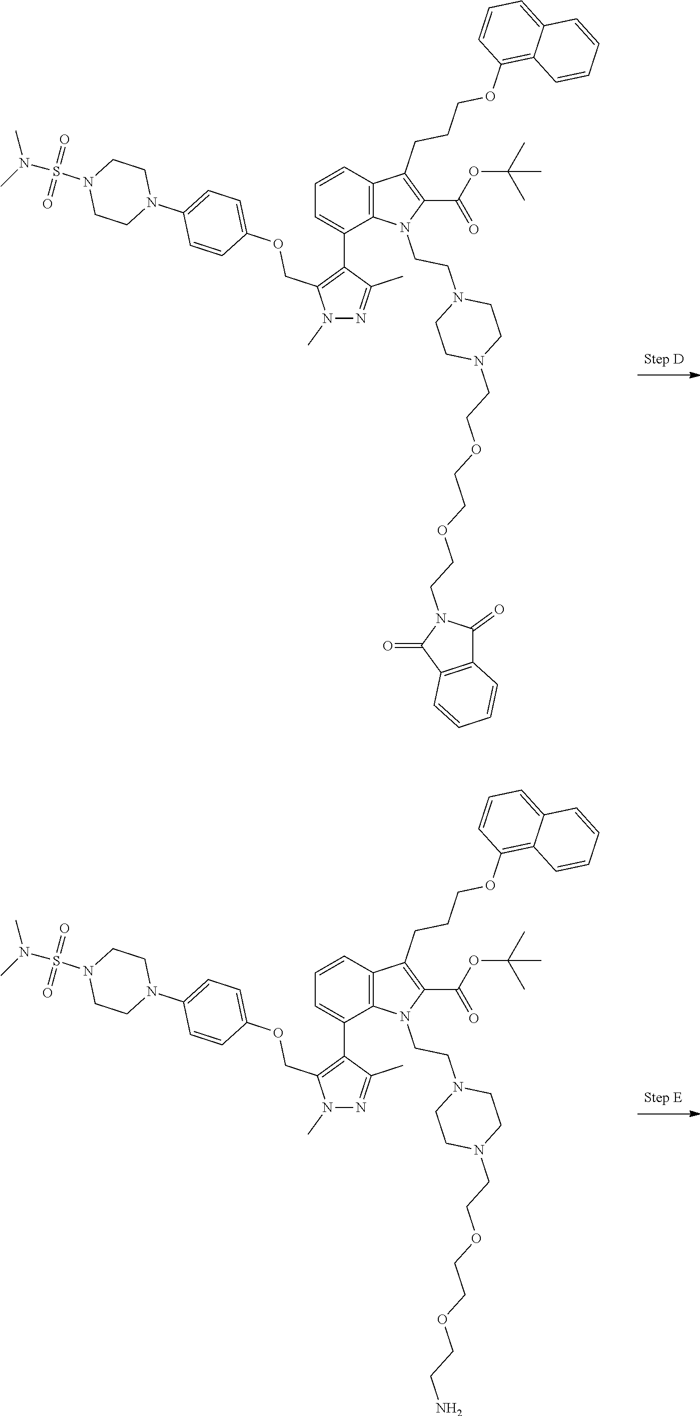 [SMILES: Cc1nn(C)c(COc2ccc(N3CCN(S(=O)(=O)N(C)C)CC3)cc2)c1-c1cccc2c(CCCOc3cccc4ccccc34)c(C(=O)OC(C)(C)C)n(CCN3CCN(CCOCCOCCN4C(=O)c5ccccc5C4=O)CC3)c12 ; Cc1nn(C)c(COc2ccc(N3CCN(S(=O)(=O)N(C)C)CC3)cc2)c1-c1cccc2c(CCCOc3cccc4ccccc34)c(C(=O)OC(C)(C)C)n(CCN3CCN(CCOCCOCCN)CC3)c12]

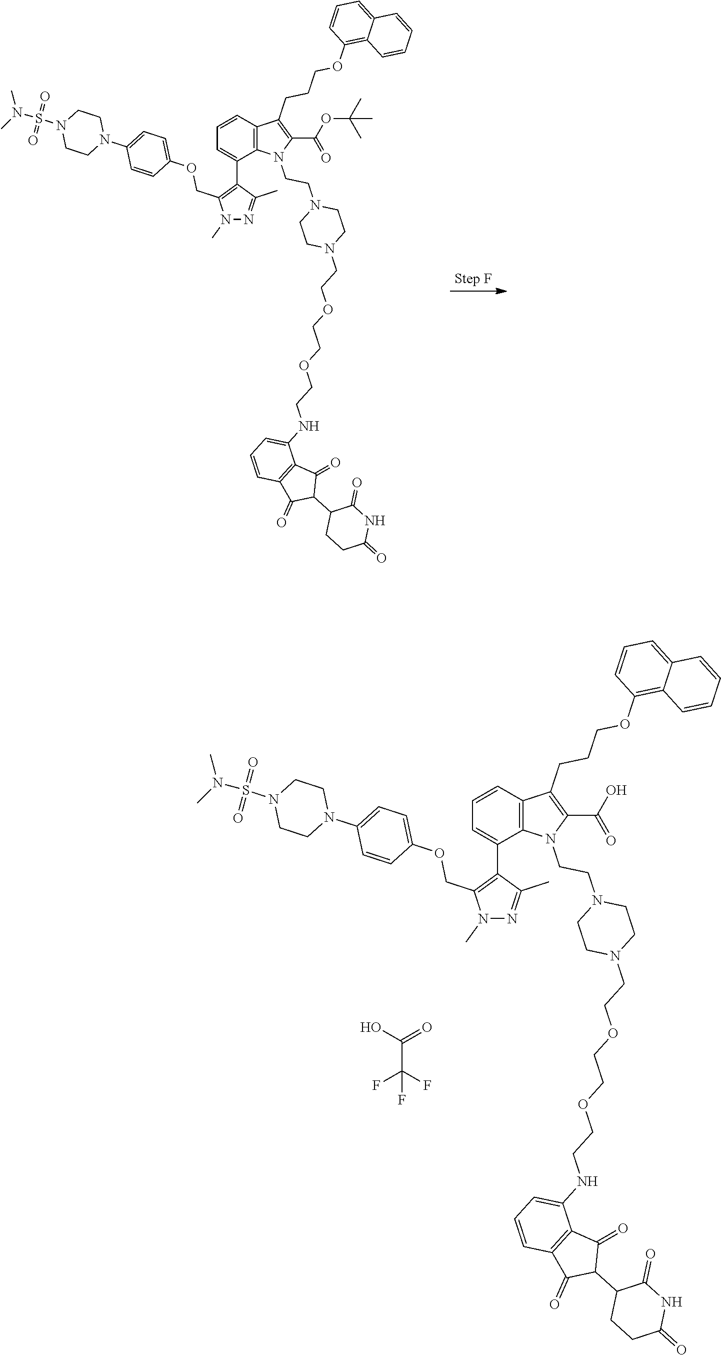 [SMILES: Cc1nn(C)c(COc2ccc(N3CCN(S(=O)(=O)N(C)C)CC3)cc2)c1-c1cccc2c(CCCOc3cccc4ccccc34)c(C(=O)OC(C)(C)C)n(CCN3CCN(CCOCCOCCNc4cccc5c4C(=O)C(C4CCC(=O)NC4=O)C5=O)CC3)c12 ; Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(CCOCCOCCNc4cccc5c4C(=O)C(C4CCC(=O)NC4=O)C5=O)CC3)c12 ; O=C(O)C(F)(F)F]

### Step A

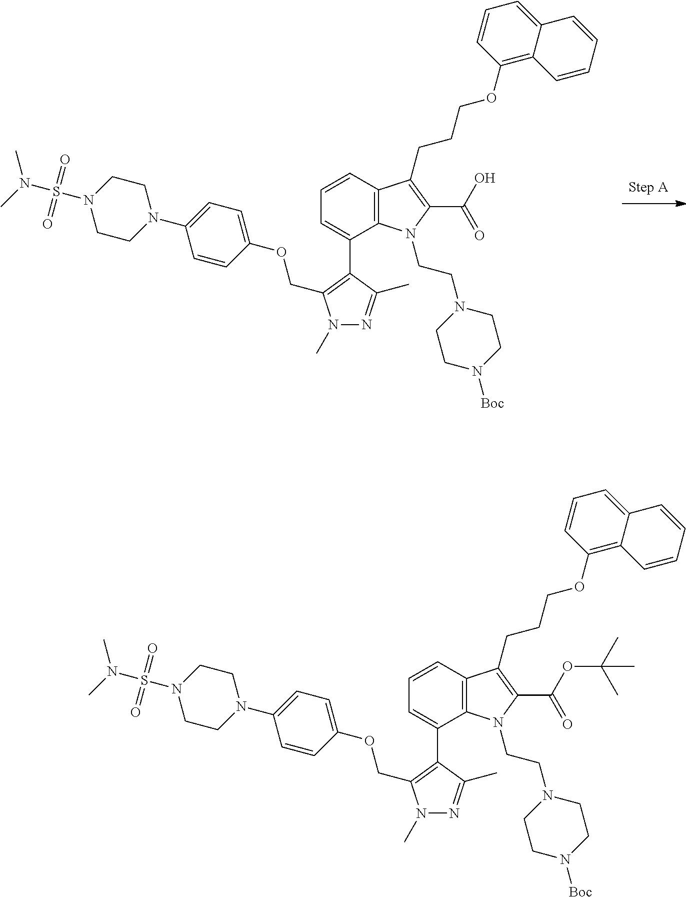 [SMILES: Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(C(=O)OC(C)(C)C)CC3)c12 ; Cc1nn(C)c(COc2ccc(N3CCN(S(=O)(=O)N(C)C)CC3)cc2)c1-c1cccc2c(CCCOc3cccc4ccccc34)c(C(=O)OC(C)(C)C)n(CCN3CCN(C(=O)OC(C)(C)C)CC3)c12]

1-(2-(4-(tert-butoxycarbonyl)piperazin-1-yl)ethyl)-7-(5-((4-(4-(N,N-dimethylsulfamoyl)piperazin-1-yl)phenoxy)methyl)-1,3-dimethyl-1H-pyrazol-4-yl)-3-(3-(naphthalen-1-yloxy)propyl)-1H-indole-2-carboxylic acid (2.0 g, 2.1 mmol) was suspended in toluene (20 ml) and the mixture was heated to reflux under nitrogen. N,N-dimethylformamide di-tert-butyl acetal (2.5 ml, 10.5 mmol) was added drop wise to the refluxing mixture. Refluxing was continued for an additional 12 h under nitrogen. After 16 h another 1.3 ml (5.3 mmol) of N,N-dimethylformamide di-tert-butyl acetal was added to it and the reaction was continued for another 12 h. Reaction mixture was then diluted with EtOAc, washed successively with NaHCO₃ (aqueous saturated solution), water and brine. Organic layer was dried over Na₂SO₄ and evaporated under reduced pressure to get the crude compound, which was then purified by flash chromatography (SiO₂, 50% EtOAc in DCM) to obtain of tert-butyl 1-(2-(4-(tert-butoxycarbonyl)piperazin-1-yl)ethyl)-7-(5-((4-(4-(N,N-dimethylsulfamoyl)piperazin-1-yl)phenoxy)methyl)-1,3-dimethyl-1H-pyrazol-4-yl)-3-(3-(naphthalen-1-yloxy)propyl)-1H-indole-2-carboxylate (1.6 g, 1.6 mmol, 76%) as yellow sticky solid.

LCMS (ESI+): m/z 1005.6 [M+H]⁺

### Step B

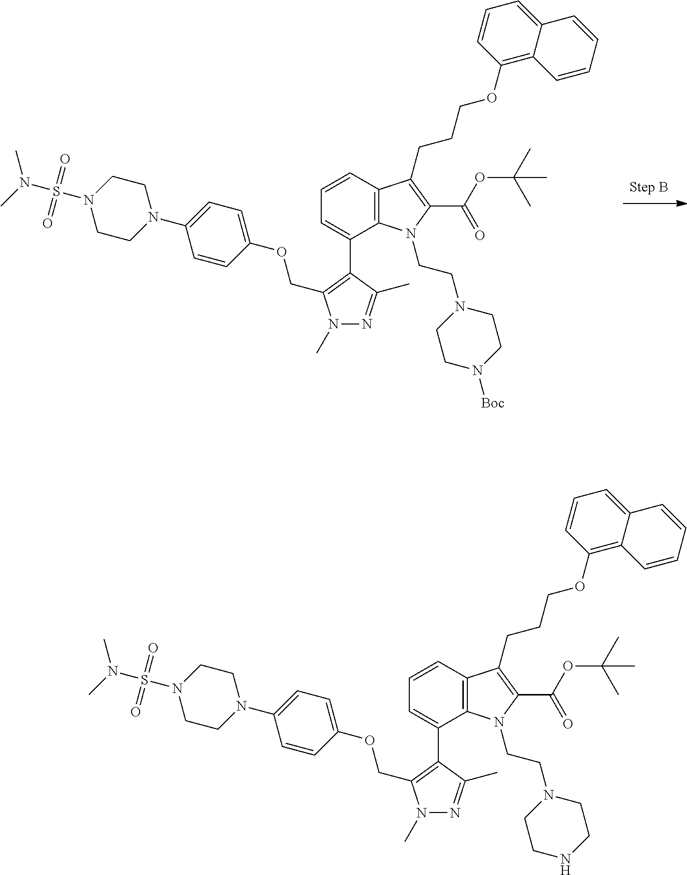 [SMILES: Cc1nn(C)c(COc2ccc(N3CCN(S(=O)(=O)N(C)C)CC3)cc2)c1-c1cccc2c(CCCOc3cccc4ccccc34)c(C(=O)OC(C)(C)C)n(CCN3CCN(C(=O)OC(C)(C)C)CC3)c12 ; Cc1nn(C)c(COc2ccc(N3CCN(S(=O)(=O)N(C)C)CC3)cc2)c1-c1cccc2c(CCCOc3cccc4ccccc34)c(C(=O)OC(C)(C)C)n(CCN3CCNCC3)c12]

To a stirred solution of compound tert-butyl 1-(2-(4-(tert-butoxycarbonyl)piperazin-1-yl)ethyl)-7-(5-((4-(4-(N,N-dimethylsulfamoyl)piperazin-1-yl)phenoxy)methyl)-1,3-dimethyl-1H-pyrazol-4-yl)-3-(3-(naphthalen-1-yloxy)propyl)-1H-indole-2-carboxylate (500 mg, 0.5 mmol) in dioxane (2 mL) was added 4M HCl in dioxane at 0° C. under nitrogen atmosphere. The reaction mixture was allowed to stir at room temperature for 1 h. The reaction mixture was quenched by slow addition of aqueous 1M NaOH at 0° C. adjusting the pH to 7. Reaction product was extracted with 3×DCM, dried over Na₂SO₄, filtered, and concentrated in vacuo to afford the crude material which was further purified by triturating it with ether and pentane to get tert-butyl 7-(5-((4-(4-(N,N-dimethylsulfamoyl)piperazin-1-yl)phenoxy)methyl)-1,3-dimethyl-1H-pyrazol-4-yl)-3-(3-(naphthalen-1-yloxy)propyl)-1-(2-(piperazin-1-yl)ethyl)-1H-indole-2-carboxylate (300 mg, 1.52 mmol, 68%) as white solid.

LCMS (ESI+): m/z 905.7 [M+H]⁺

### Step C

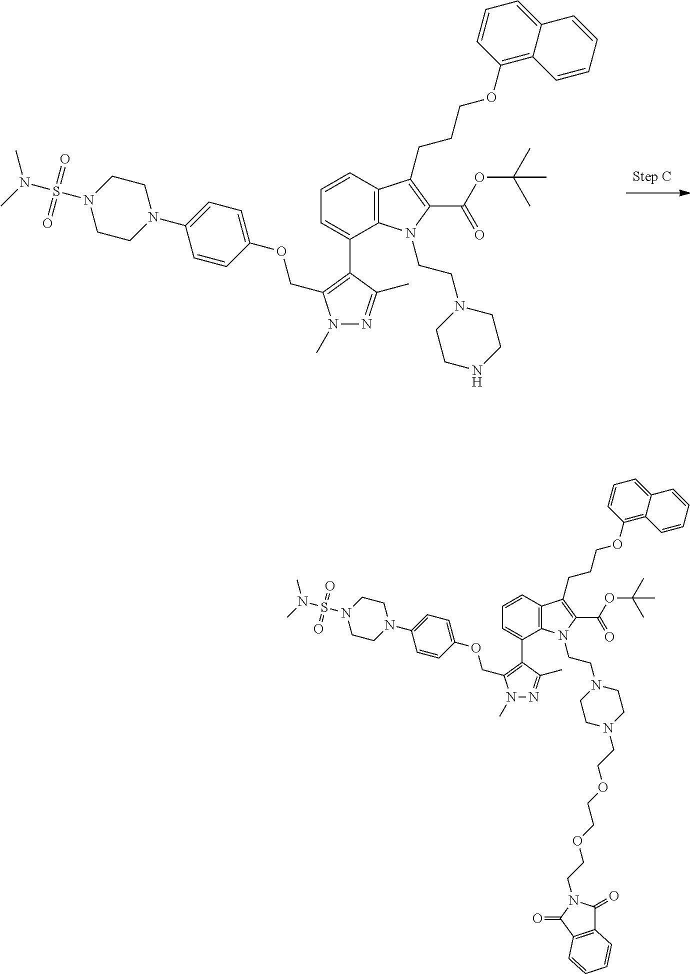 [SMILES: Cc1nn(C)c(COc2ccc(N3CCN(S(=O)(=O)N(C)C)CC3)cc2)c1-c1cccc2c(CCCOc3cccc4ccccc34)c(C(=O)OC(C)(C)C)n(CCN3CCNCC3)c12 ; Cc1nn(C)c(COc2ccc(N3CCN(S(=O)(=O)N(C)C)CC3)cc2)c1-c1cccc2c(CCCOc3cccc4ccccc34)c(C(=O)OC(C)(C)C)n(CCN3CCN(CCOCCOCCN4C(=O)c5ccccc5C4=O)CC3)c12]

To a solution of tert-butyl 7-(5-((4-(4-(N,N-dimethylsulfamoyl)piperazin-1-yl)phenoxy)methyl)-1,3-dimethyl-1H-pyrazol-4-yl)-3-(3-(naphthalen-1-yloxy)propyl)-1-(2-(piperazin-1-yl)ethyl)-1H-indole-2-carboxylate (600 mg, 0.66 mmol), Et₃N (0.465 ml, 3.32 mmol) and KI (198 mg, 1.19 mmol) in DMF (12 ml) 2-(2-(2-(3-bromopropoxy)ethoxy)ethyl)isoindoline-1,3-dione (401.2 mg, 1.13 mmol) was added and the reaction mixture was allowed to stir at 60° C. for 16 h under nitrogen. The reaction mixture was evaporated under reduced pressure to get the crude compound, which was then purified by flash chromatography (SiO2, 5% MeOH in DCM) to get tert-butyl 7-(5-((4-(4-(N,N-dimethylsulfamoyl)piperazin-1-yl)phenoxy)methyl)-1,3-dimethyl-1H-pyrazol-4-yl)-1-(2-(4-(3-(2-(2-(1,3-dioxoisoindolin-2-yl)ethoxy)ethoxy)propyl)piperazin-1-yl)ethyl)-3-(3-(naphthalen-1-yloxy)propyl)-1H-indole-2-carboxylate (720 mg, 0.61 mmol, 92%) as yellowish liquid.

LCMS (ESI+): m/z 1181.4 [M+H]⁺

### Step D

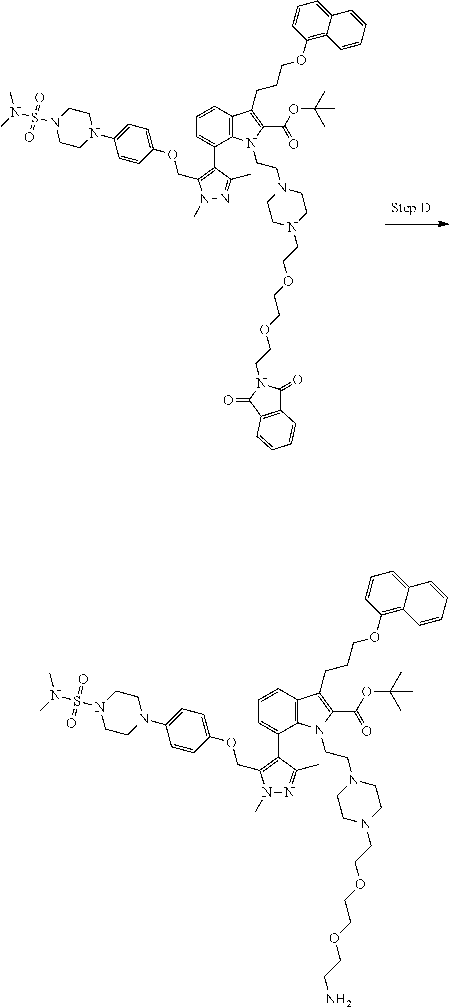 [SMILES: Cc1nn(C)c(COc2ccc(N3CCN(S(=O)(=O)N(C)C)CC3)cc2)c1-c1cccc2c(CCCOc3cccc4ccccc34)c(C(=O)OC(C)(C)C)n(CCN3CCN(CCOCCOCCN4C(=O)c5ccccc5C4=O)CC3)c12 ; Cc1nn(C)c(COc2ccc(N3CCN(S(=O)(=O)N(C)C)CC3)cc2)c1-c1cccc2c(CCCOc3cccc4ccccc34)c(C(=O)OC(C)(C)C)n(CCN3CCN(CCOCCOCCN)CC3)c12]

To a stirred solution of tert-butyl 7-(5-((4-(4-(N,N-dimethylsulfamoyl)piperazin-1-yl)phenoxy)methyl)-1,3-dimethyl-1H-pyrazol-4-yl)-1-(2-(4-(3-(2-(2-(1,3-dioxoisoindolin-2-yl)ethoxy)ethoxy)propyl)piperazin-1-yl)ethyl)-3-(3-(naphthalen-1-yloxy)propyl)-1H-indole-2-carboxylate (720 mg, 0.610 mmol) in tert-butanol (30 mL), hydrazine hydrate (5.0 mL) was added. Reaction mixture was heated to 90° C. for 16 h in a sealed tube. The reaction mixture was evaporated under reduced pressure in order to obtain the crude compound, which was then purified by preparative HPLC to afford tert-butyl 1-(2-(4-(3-(2-(2-aminoethoxy)ethoxy)propyl)piperazin-1-yl)ethyl)-7-(5-((4-(4-(N,N-dimethylsulfamoyl)piperazin-1-yl)phenoxy)methyl)-1,3-dimethyl-1H-pyrazol-4-yl)-3-(3-(naphthalen-1-yloxy)propyl)-1H-indole-2-carboxylate (240 mg, 0.23 mmol, 37%) as white solid.

LCMS (ESI+): m/z 1050.9 [M+H]⁺

### Step E

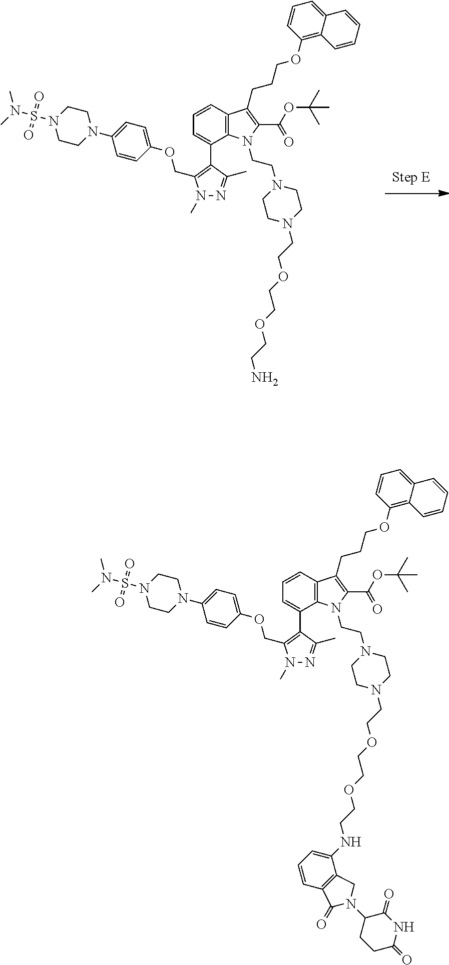 [SMILES: Cc1nn(C)c(COc2ccc(N3CCN(S(=O)(=O)N(C)C)CC3)cc2)c1-c1cccc2c(CCCOc3cccc4ccccc34)c(C(=O)OC(C)(C)C)n(CCN3CCN(CCOCCOCCN)CC3)c12 ; Cc1nn(C)c(COc2ccc(N3CCN(S(=O)(=O)N(C)C)CC3)cc2)c1-c1cccc2c(CCCOc3cccc4ccccc34)c(C(=O)OC(C)(C)C)n(CCN3CCN(CCOCCOCCNc4cccc5c4CN(C4CCC(=O)NC4=O)C5=O)CC3)c12]

To a well stirred solution of tert-butyl 1-(2-(4-(3-(2-(2-aminoethoxy)ethoxy)propyl)piperazin-1-yl)ethyl)-7-(5-((4-(4-(N,N-dimethylsulfamoyl)piperazin-1-yl)phenoxy)methyl)-1,3-dimethyl-1H-pyrazol-4-yl)-3-(3-(naphthalen-1-yloxy)propyl)-1H-indole-2-carboxylate (130 mg, 0.12 mmol) in DMSO (10.0 ml) was added 2-(2,6-dioxopiperidin-3-yl)-4-fluoroisoindoline-1,3-dione (78.6 mg, 0.28 mmol), followed by DIPEA (0.11 mL, 0.62 mmol) and the mixture was allowed to stir at 90° C. for 16 h under nitrogen. The reaction mixture was diluted with EtOAc, washed successively with cold water and brine, the organic layer was dried over Na₂SO₄ and evaporated under reduced pressure to get the crude compound, which was then purified by preparative HPLC (H₂O:MeCN+0.1% FA) to afford tert-butyl 7-(5-((4-(4-(N,N-dimethylsulfamoyl)piperazin-1-yl)phenoxy)methyl)-1,3-dimethyl-1H-pyrazol-4-yl)-1-(2-(4-(3-(2-(2-((2-(2,6-dioxopiperidin-3-yl)-1,3-dioxoisoindolin-4-yl)amino)ethoxy)ethoxy)propyl)piperazin-1-yl)ethyl)-3-(3-(naphthalen-1-yloxy)propyl)-1H-indole-2-carboxylate (49 mg, 0.038 mmol, 31%) as yellow solid.

LCMS (ESI+): m/z 1306.9 [M+H]⁺

### Step F

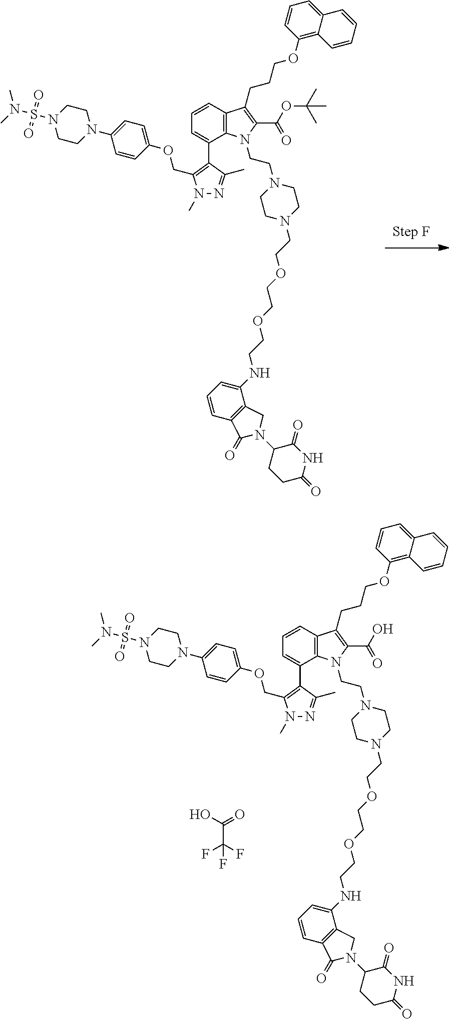 [SMILES: Cc1nn(C)c(COc2ccc(N3CCN(S(=O)(=O)N(C)C)CC3)cc2)c1-c1cccc2c(CCCOc3cccc4ccccc34)c(C(=O)OC(C)(C)C)n(CCN3CCN(CCOCCOCCNc4cccc5c4CN(C4CCC(=O)NC4=O)C5=O)CC3)c12 ; Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(CCOCCOCCNc4cccc5c4CN(C4CCC(=O)NC4=O)C5=O)CC3)c12 ; O=C(O)C(F)(F)F]

To a well stirred solution of tert-butyl 7-(5-((4-(4-(N,N-dimethylsulfamoyl)piperazin-1-yl)phenoxy)methyl)-1,3-dimethyl-1H-pyrazol-4-yl)-1-(2-(4-(3-(2-(2-((2-(2,6-dioxopiperidin-3-yl)-1,3-dioxoisoindolin-4-yl)amino)ethoxy)ethoxy)propyl)piperazin-1-yl)ethyl)-3-(3-(naphthalen-1-yloxy)propyl)-1H-indole-2-carboxylate (34 mg, 0.026 mmol) in DCM (4 mL) was added TFA (4 mL) and the mixture was allowed to stir at room temperature for 16 h under nitrogen. After complete consumption of the starting material (monitored by LCMS), the volatiles were evaporated under reduced pressure to get the crude compound which was triturated using diethyl ether to afford 7-(5-((4-(4-(N,N-dimethylsulfamoyl)piperazin-1-yl)phenoxy)methyl)-1,3-dimethyl-1H-pyrazol-4-yl)-1-(2-(4-(3-(2-(2-((2-(2,6-dioxopiperidin-3-yl)-1,3-dioxoisoindolin-4-yl)amino)ethoxy)ethoxy)propyl)piperazin-1-yl)ethyl)-3-(3-(naphthalen-1-yloxy)propyl)-1H-indole-2-carboxylic acid trifluoroacetate (30 mg, 0.024 mmol, 92%) as yellow solid.

LCMS (ESI+): m/z 1250.7 [M+H]⁺

¹H NMR (400 MHz, DMSO) δ 11.10 (s, 1H), 9.27 (s, 1H), 8.27-8.17 (m, 1H), 7.86 (dd, J=6.6, 2.7 Hz, 1H), 7.75 (d, J=8.0 Hz, 1H), 7.58 (dd, J=8.6, 7.1 Hz, 1H), 7.54-7.47 (m, 2H), 7.45 (d, J=8.2 Hz, 1H), 7.37 (t, J=7.9 Hz, 1H), 7.13 (d, J=8.5 Hz, 1H), 7.10-7.03 (m, 2H), 6.90 (dd, J=13.9, 7.4 Hz, 2H), 6.84-6.74 (m, 4H), 6.59 (t, J=5.8 Hz, 1H), 5.05 (dd, J=12.7, 5.4 Hz, 1H), 4.90-4.78 (m, 2H), 4.53 (s, 1H), 4.20 (t, J=6.2 Hz, 3H), 3.87 (s, 3H), 3.63-3.27 (m, 15H, overlaps with water), 3.24 (dd, J=6.4, 3.5 Hz, 4H), 3.07-2.96 (m, 6H), 2.92-2.79 (m, 2H), 2.77 (s, 6H), 2.63-2.53 (m, 2H), 2.26-2.08 (m, 6H), 2.05-1.92 (m, 4H), 1.88-1.77 (m, 2H).

### Example 41. 7-(5-((4-(4-(N,N-dimethylsulfamoyl)piperazin-1-yl)phenoxy)methyl)-1,3-dimethyl-1H-pyrazol-4-yl)-1-(2-(4-(2-((2-(2,6-dioxopiperidin-3-yl)-1,3-dioxoisoindolin-5-yl)oxy)acetyl)piperazin-1-yl)ethyl)-3-(3-(naphthalen-1-yloxy)propyl)-1H-indole-2-carboxylic acid (248)

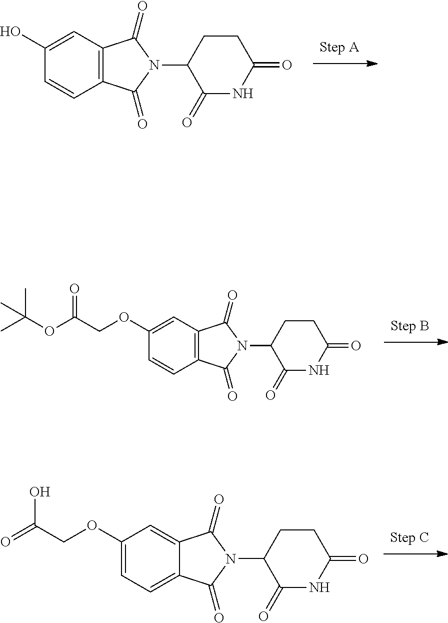 [SMILES: O=C1CCC(N2C(=O)c3ccc(O)cc3C2=O)C(=O)N1 ; CC(C)(C)OC(=O)COc1ccc2c(c1)C(=O)N(C1CCC(=O)NC1=O)C2=O ; O=C(O)COc1ccc2c(c1)C(=O)N(C1CCC(=O)NC1=O)C2=O]

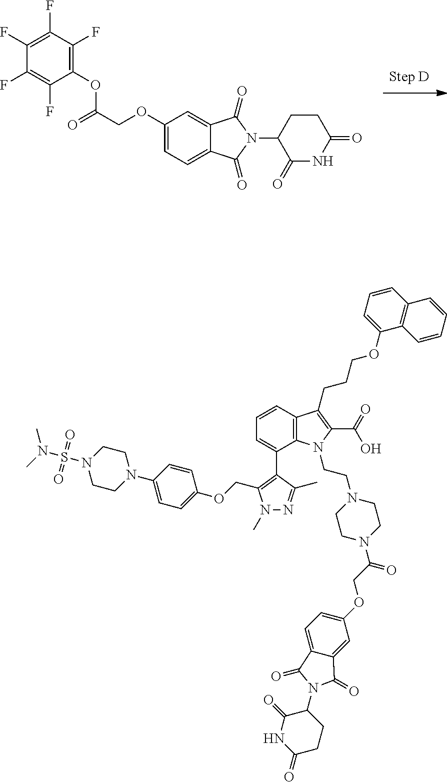 [SMILES: O=C1CCC(N2C(=O)c3ccc(OCC(=O)Oc4c(F)c(F)c(F)c(F)c4F)cc3C2=O)C(=O)N1 ; Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(C(=O)COc4ccc5c(c4)C(=O)N(C4CCC(=O)NC4=O)C5=O)CC3)c12]

### Step A

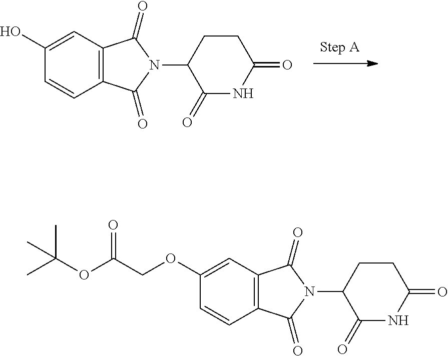 [SMILES: O=C1CCC(N2C(=O)c3ccc(O)cc3C2=O)C(=O)N1 ; CC(C)(C)OC(=O)COc1ccc2c(c1)C(=O)N(C1CCC(=O)NC1=O)C2=O]

To a solution of 2-(2,6-dioxopiperidin-3-yl)-5-hydroxyisoindoline-1,3-dione (0.2 g, 0.73 mmol) in DMF (2 ml) was added potassium iodide (60.584 mg, 0.365 mmol) and potassium bicarbonate (145.985 mg, 1.46 mmol) followed by tert-butyl bromo acetate (140.0 mg, 0.73 mmol) and the resulting reaction mixture was allowed to stir at 60° C. for 16 h under nitrogen. After complete consumption of the starting material the reaction mixture was diluted with ethyl acetate and washed successively with cold water and brine. Organic layer was dried over sodium sulphate and evaporated under reduced pressure to give the crude compound which was then triturated with diethyl ether to afford tert-butyl 2-((2-(2,6-dioxopiperidin-3-yl)-1,3-dioxoisoindolin-5-yl)oxy)acetate (260 mg, 0.670 mmol, 91.72%) as white solid.

LCMS (ESI+): m/z 388.8 [M+H]⁺

### Step B

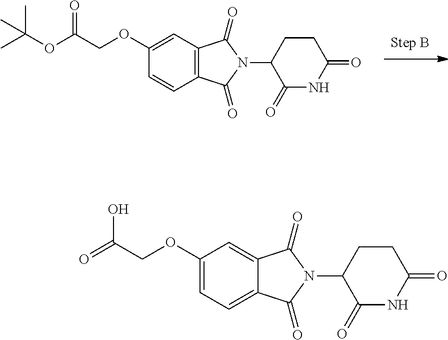 [SMILES: CC(C)(C)OC(=O)COc1ccc2c(c1)C(=O)N(C1CCC(=O)NC1=O)C2=O ; O=C(O)COc1ccc2c(c1)C(=O)N(C1CCC(=O)NC1=O)C2=O]

To a suspension of tert-butyl 2-((2-(2,6-dioxopiperidin-3-yl)-1,3-dioxoisoindolin-5-yl)oxy)acetate (0.1 g, 0.258 mmol) in DCM (3 ml) was added TFA (1 ml) dropwise at 0° C. under nitrogen. The mixture was allowed to stir at RT for 16 h. After complete consumption of the starting material the volatiles were evaporated under reduced pressure to get the crude compound which was triturated with diethyl ether to afford 2-((2-(2,6-dioxopiperidin-3-yl)-1,3-dioxoisoindolin-5-yl)oxy)acetic acid (58 mg, 0.174 mmol, 67.73%) as off white solid.

LCMS (ESI−): m/z 330.9 [M−H]⁻

### Step C

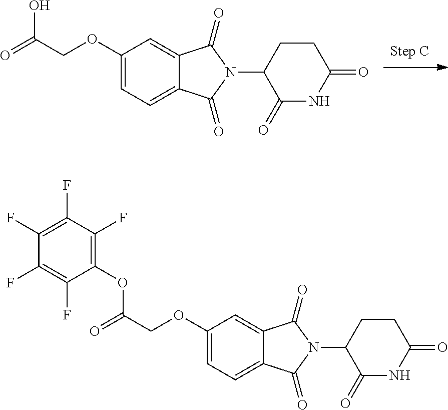 [SMILES: O=C(O)COc1ccc2c(c1)C(=O)N(C1CCC(=O)NC1=O)C2=O ; O=C1CCC(N2C(=O)c3ccc(OCC(=O)Oc4c(F)c(F)c(F)c(F)c4F)cc3C2=O)C(=O)N1]

To a well stirred solution of 2-((2-(2,6-dioxopiperidin-3-yl)-1,3-dioxoisoindolin-5-yl)oxy)acetic acid (200 mg, 0.6 mmol) in DMF (2 ml) was added DIPEA (1 ml, 6 mmol) and the reaction mixture was cooled to 0° C. Pentafluorophenyl trifluoroacetate (843 mg, 3 mmol) was added and the mixture was allowed to stir at RT for 2 h under nitrogen. After complete consumption of the starting material (monitored by LCMS) the volatiles were evaporated under reduced pressure to obtain 200 mg of crude perfluorophenyl 2-((2-(2,6-dioxopiperidin-3-yl)-1,3-dioxoisoindolin-5-yl)oxy)acetate as brown sticky solid which was used for the next step without further purification.

### Step D

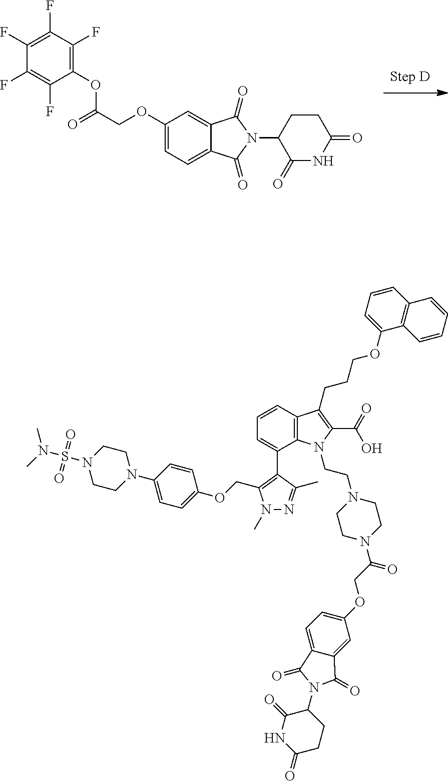 [SMILES: O=C1CCC(N2C(=O)c3ccc(OCC(=O)Oc4c(F)c(F)c(F)c(F)c4F)cc3C2=O)C(=O)N1 ; Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(C(=O)COc4ccc5c(c4)C(=O)N(C4CCC(=O)NC4=O)C5=O)CC3)c12]

To a solution of perfluorophenyl 2-((2-(2,6-dioxopiperidin-3-yl)-1,3-dioxoisoindolin-5-yl)oxy)acetate (200 mg, crude) in DMF (2 mL) were added DIPEA (85 μL, 0.7 mmol) and a solution of 7-(5-((4-(4-(N,N-dimethylsulfamoyl)piperazin-1-yl)phenoxy)methyl)-1,3-dimethyl-1H-pyrazol-4-yl)-3-(3-(naphthalen-1-yloxy)propyl)-1-(2-(piperazin-1-yl)ethyl)-1H-indole-2-carboxylic acid (120 mg, 0.14 mmol) in DMF (1 ml) at 0° C. under nitrogen. The reaction mixture was allowed to stir at ambient temperature for 16 h. After complete consumption of starting material (monitored by LCMS), the reaction mixture was concentrated in vacuo and purified by reverse phase preparative HPLC (H₂O:MeCN+0.1% FA) to obtain 7-(5-((4-(4-(N,N-dimethylsulfamoyl)piperazin-1-yl)phenoxy)methyl)-1,3-dimethyl-1H-pyrazol-4-yl)-1-(2-(4-(2-((2-(2,6-dioxopiperidin-3-yl)-1,3-dioxoisoindolin-5-yl)oxy)acetyl)piperazin-1-yl)ethyl)-3-(3-(naphthalen-1-yloxy)propyl)-1H-indole-2-carboxylic acid (40 mg, 0.034 mmol, 24%) as off white solid.

LCMS (ESI+): m/z 1163.7 [M+H]⁺

¹H NMR (400 MHz, DMSO) δ 13.21 (bs, 1H), 11.10 (s, 1H), 8.27-8.17 (m, 1H), 7.88-7.83 (m, 1H), 7.81 (d, J=8.3 Hz, 1H), 7.73 (d, J=8.0 Hz, 1H), 7.54-7.46 (m, 2H), 7.39 (dt, J=18.6, 8.1 Hz, 3H), 7.30 (dd, J=8.3, 2.1 Hz, 1H), 7.06 (t, J=7.6 Hz, 1H), 6.89 (dd, J=13.1, 7.3 Hz, 2H), 6.85-6.71 (m, 4H), 5.11 (dd, J=12.8, 5.4 Hz, 1H), 5.01 (s, 2H), 4.86 (q, J=11.9 Hz, 2H), 4.63-4.48 (m, 1H), 4.20 (t, J=6.1 Hz, 3H), 3.87 (s, 3H), 3.29-3.25 (m, 5H), 3.24-3.21 (m, 4H), 3.01-2.95 (m, 4H), 2.94-2.83 (m, 1H), 2.75 (s, 6H), 2.64-2.52 (m, 2H), 2.21 (p, J=7.3, 6.9 Hz, 2H), 2.17-1.98 (m, 8H), 1.97 (s, 3H).

### Example 42. 7-(5-((4-(4-(N,N-dimethylsulfamoyl)piperazin-1-yl)phenoxy)methyl)-1,3-dimethyl-1H-pyrazol-4-yl)-1-(2-(4-(2-((2-(2,6-dioxopiperidin-3-yl)-1-oxoisoindolin-4-yl)oxy)acetyl)piperazin-1-yl)ethyl)-3-(3-(naphthalen-1-yloxy)propyl)-1H-indole-2-carboxylic acid (249)

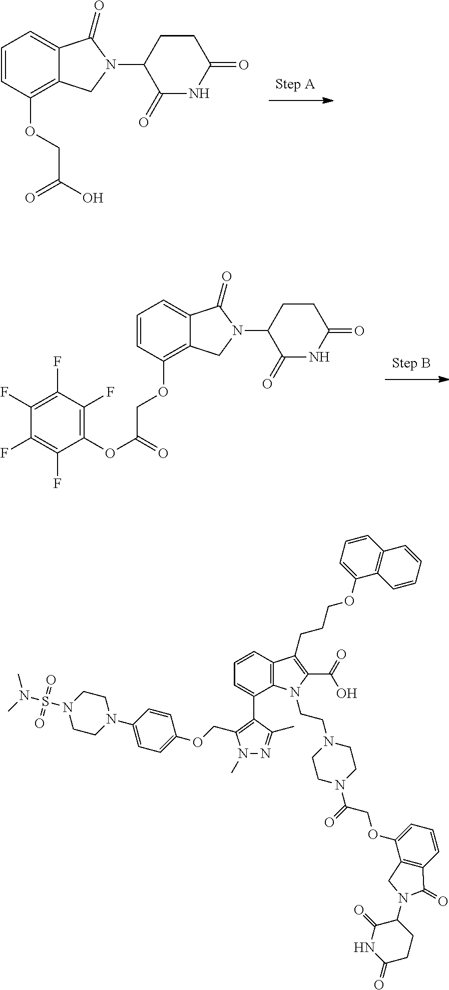 [SMILES: O=C(O)COc1cccc2c1CN(C1CCC(=O)NC1=O)C2=O ; O=C1CCC(N2Cc3c(OCC(=O)Oc4c(F)c(F)c(F)c(F)c4F)cccc3C2=O)C(=O)N1 ; Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(C(=O)COc4cccc5c4CN(C4CCC(=O)NC4=O)C5=O)CC3)c12]

### Step A

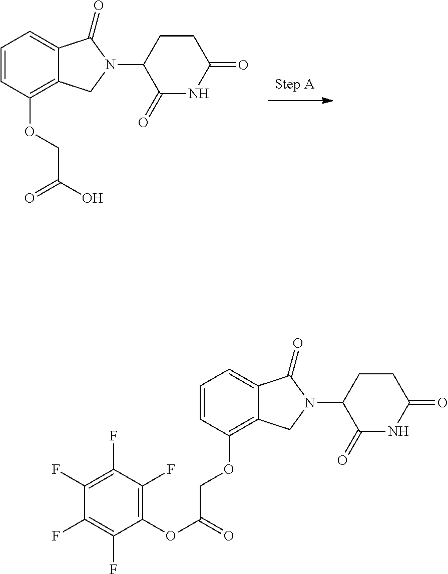 [SMILES: O=C(O)COc1cccc2c1CN(C1CCC(=O)NC1=O)C2=O ; O=C1CCC(N2Cc3c(OCC(=O)Oc4c(F)c(F)c(F)c(F)c4F)cccc3C2=O)C(=O)N1]

To a solution of 2-((2-(2,6-dioxopiperidin-3-yl)-1-oxoisoindolin-4-yl)oxy)acetic acid (200 mg, 0.63 mmol) in DMF (2 ml) DIPEA (1 ml, 6 mmol) was added. The reaction mixture was cooled to 0° C., to it was added pentafluorophenyl trifluoroacetate (843 mg, 3 mmol) and the mixture was allowed to stir at ambient temperature for 2 h under nitrogen. After complete consumption of the starting material (monitored by LCMS) the volatiles are evaporated under reduced pressure to get 200 mg of crude perfluorophenyl 2-((2-(2,6-dioxopiperidin-3-yl)-1-oxoisoindolin-4-yl)oxy)acetate as brown sticky solid which was used for the next step without further purification.

### Step B

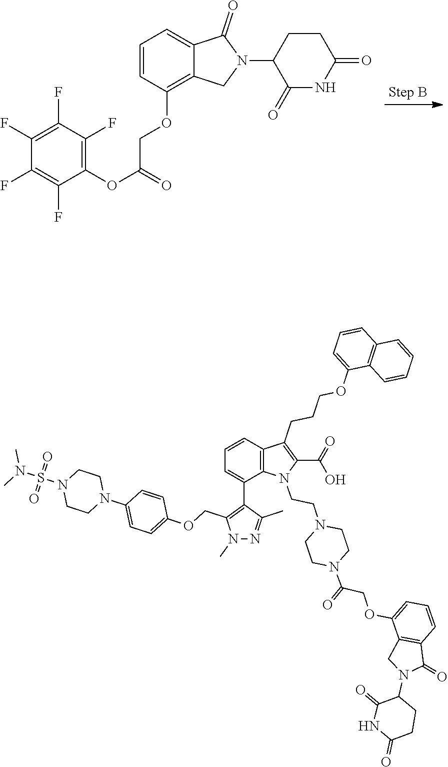 [SMILES: O=C1CCC(N2Cc3c(OCC(=O)Oc4c(F)c(F)c(F)c(F)c4F)cccc3C2=O)C(=O)N1 ; Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(C(=O)COc4cccc5c4CN(C4CCC(=O)NC4=O)C5=O)CC3)c12]

To a solution of perfluorophenyl 2-((2-(2,6-dioxopiperidin-3-yl)-1-oxoisoindolin-4-yl)oxy)acetate (200 mg, crude) in DMF (2 ml) were added DIPEA (85 μL, 0.7 mmol) and a solution of 7-(5-((4-(4-(N,N-dimethylsulfamoyl)piperazin-1-yl)phenoxy)methyl)-1,3-dimethyl-1H-pyrazol-4-yl)-3-(3-(naphthalen-1-yloxy)propyl)-1-(2-(piperazin-1-yl)ethyl)-1H-indole-2-carboxylic acid (120 mg, 0.142 mmol) in DMF (1 ml) successively at 0° C. under nitrogen. The reaction mixture was allowed to stir at ambient temperature for 16 h. The reaction mixture was concentrated in vacuo and purified by preparative HPLC (H₂O:MeCN+0.1% FA) to get 7-(5-((4-(4-(N,N-dimethylsulfamoyl)piperazin-1-yl)phenoxy)methyl)-1,3-dimethyl-1H-pyrazol-4-yl)-1-(2-(4-(2-((2-(2,6-dioxopiperidin-3-yl)-1-oxoisoindolin-4-yl)oxy)acetyl)piperazin-1-yl)ethyl)-3-(3-(naphthalen-1-yloxy)propyl)-1H-indole-2-carboxylic acid (45 mg, 0.039 mmol, 28%) as off white solid.

LCMS (ESI+): m/z 1149.7 [M+H]⁺

¹H NMR (400 MHz, DMSO) δ 13.27 (s, 1H), 10.97 (s, 1H), 8.26-8.17 (m, 1H), 7.90-7.80 (m, 1H), 7.72 (d, J=8.0 Hz, 1H), 7.56-7.46 (m, 2H), 7.46-7.33 (m, 3H), 7.31 (d, J=7.5 Hz, 1H), 7.14-7.01 (m, 2H), 6.96-6.85 (m, 2H), 6.85-6.75 (m, 4H), 5.10 (dd, J=13.1, 4.9 Hz, 1H), 4.97-4.79 (m, 4H), 4.62-4.48 (m, 1H), 4.42-4.31 (m, 1H), 4.28-4.13 (m, 4H), 3.85 (d, J=4.0 Hz, 3H), 3.30-3.29 (m, 8H), 3.24-3.20 (m, 4H), 3.02-2.93 (m, 5H), 2.93-2.84 (m, 1H), 2.62-2.52 (m, 2H), 2.45-2.36 (m, 1H), 2.36-2.30 (m, 1H), 2.26-2.15 (m, 2H), 2.15-1.97 (m, 8H), 1.96 (s, 3H).

### Example 43. 7-(3-((4-Bromo-3-formylphenoxy)methyl)-1,5-dimethyl-1H-pyrazol-4-yl)-1-(2-(4-(2-((2-(2,6-dioxopiperidin-3-yl)-1,3-dioxoisoindolin-4-yl)oxy)acetyl)piperazin-1-yl)ethyl)-3-(3-(naphthalen-1-yloxy)propyl)-1H-indole-2-carboxylic acid (251)

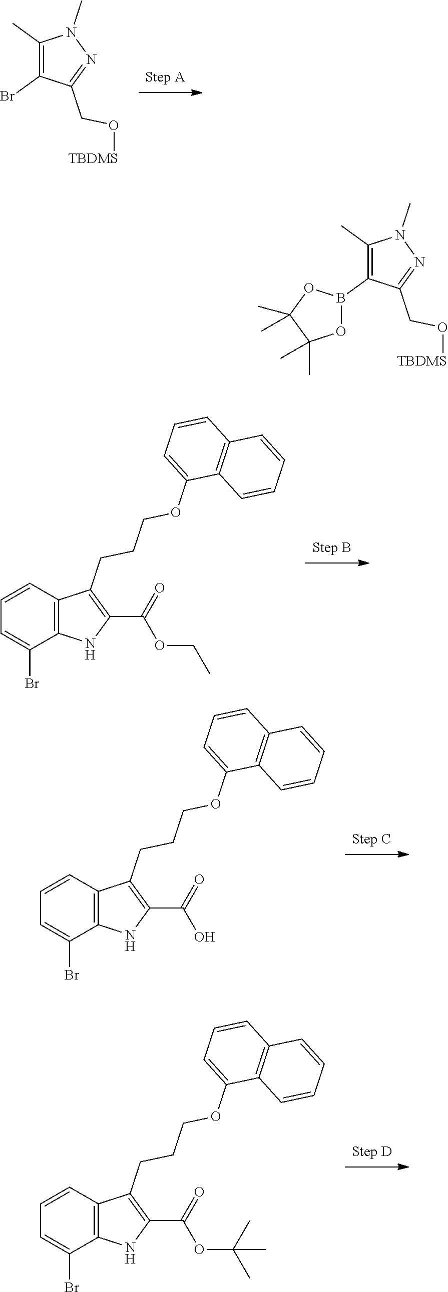

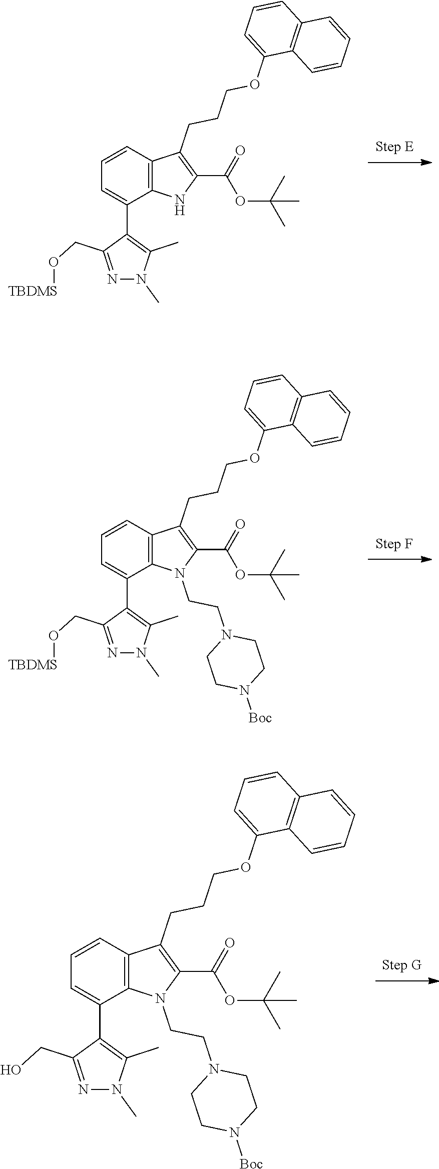

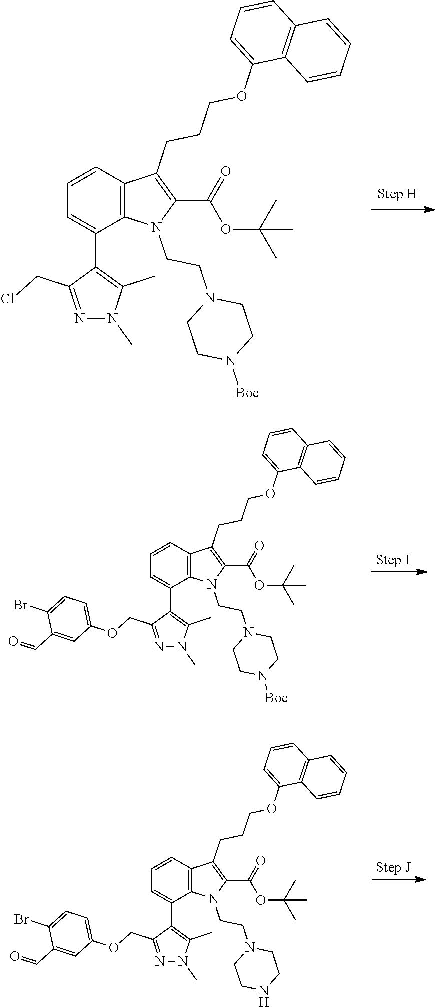 [SMILES: Cc1c(-c2cccc3c(CCCOc4cccc5ccccc45)c(C(=O)OC(C)(C)C)n(CCN4CCN(C(=O)OC(C)(C)C)CC4)c23)c(CCl)nn1C ; Cc1c(-c2cccc3c(CCCOc4cccc5ccccc45)c(C(=O)OC(C)(C)C)n(CCN4CCN(C(=O)OC(C)(C)C)CC4)c23)c(COc2ccc(Br)c(C=O)c2)nn1C ; Cc1c(-c2cccc3c(CCCOc4cccc5ccccc45)c(C(=O)OC(C)(C)C)n(CCN4CCNCC4)c23)c(COc2ccc(Br)c(C=O)c2)nn1C]

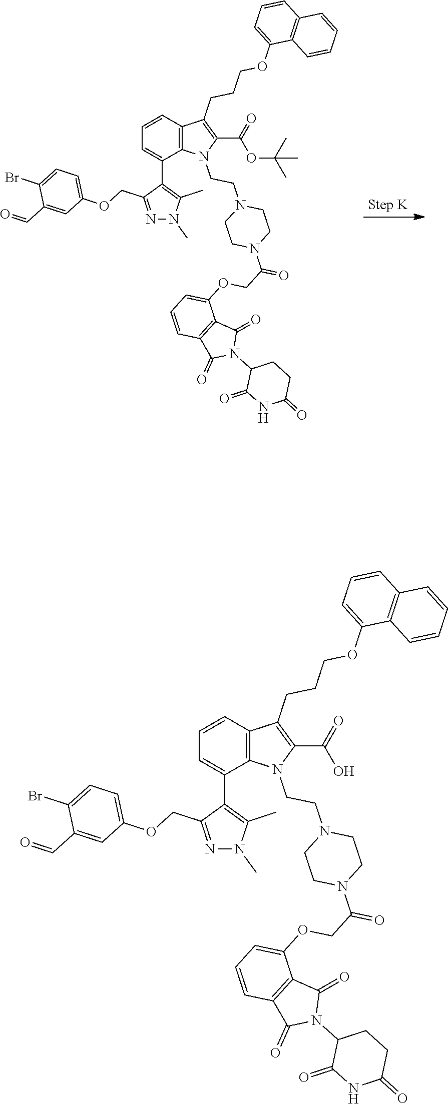 [SMILES: Cc1c(-c2cccc3c(CCCOc4cccc5ccccc45)c(C(=O)OC(C)(C)C)n(CCN4CCN(C(=O)COc5cccc6c5C(=O)N(C5CCC(=O)NC5=O)C6=O)CC4)c23)c(COc2ccc(Br)c(C=O)c2)nn1C ; Cc1c(-c2cccc3c(CCCOc4cccc5ccccc45)c(C(=O)O)n(CCN4CCN(C(=O)COc5cccc6c5C(=O)N(C5CCC(=O)NC5=O)C6=O)CC4)c23)c(COc2ccc(Br)c(C=O)c2)nn1C]

### Step A

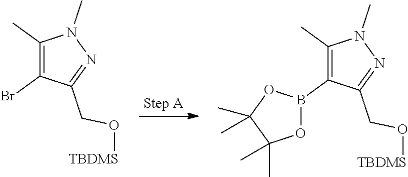

To a well stirred solution of 4-bromo-3-(((tert-butyldimethylsilyl)oxy)methyl)-1,5-dimethyl-1H-pyrazole (5 g, 15.674 mmol) in THE (120 ml) was added butyllithium (10.7 ml, 17.241 mmol, 1.6 M in hexane) at −78° C. under argon. The mixture was stirred at −78° C. for 50 min. 2-Isopropoxy-4,4,5,5-tetramethyl-1,3,2-dioxaborolane (3.5 ml, 17.241 mmol) was added to the reaction mixture. The dry ice bath was removed. The mixture was slowly warmed to RT and was stirred for 1 h. After complete consumption of the starting material (monitored by TLC and LCMS) the excess butyl lithium was quenched by addition of saturated ammonium chloride. It was then diluted with EtOAc, washed successively with water and brine, dried over Na₂SO₄ and evaporated under reduced pressure to afford 5.2 g of 3-(((tert-butyldimethylsilyl)oxy)methyl)-1,5-dimethyl-4-(4,4,5,5-tetramethyl-1,3,2-dioxaborolan-2-yl)-1H-pyrazole as white gummy solid which was used for the next step without further purification.

LCMS (ESI+): m/z 366.3 [M+H]⁺

### Step B

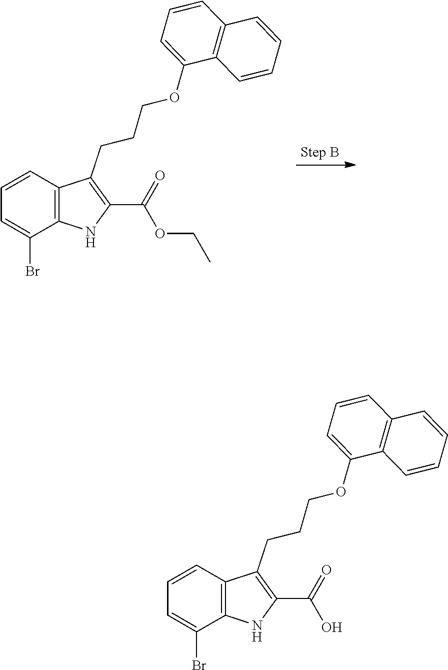 [SMILES: CCOC(=O)c1[nH]c2c(Br)cccc2c1CCCOc1cccc2ccccc12 ; O=C(O)c1[nH]c2c(Br)cccc2c1CCCOc1cccc2ccccc12]

Ethyl 7-bromo-3-(3-(naphthalen-1-yloxy)propyl)-1H-indole-2-carboxylate (7 g, 15.52 mmol) was dissolved in EtOH (220 mL) and a solution of NaOH (2.5 g, 62.08 mmol) in water (12 mL) was added to it. The mixture was heated under reflux for 16 h. After complete consumption of the starting material (monitored by TLC and LCMS) the reaction mixture was cooled down to room temperature, solvents were evaporated under reduced pressure to get the crude reaction mixture. It was then diluted with water and washed with EtOAc. Aqueous layer was carefully acidified using 1M HCl to pH=3, extracted with dichloromethane (3×50 ml), dried over Na₂SO₄, filtered, and concentrated in vacuo to afford 7-bromo-3-(3-(naphthalen-1-yloxy)propyl)-1H-indole-2-carboxylic acid (5.7 g, 13.47 mmol, 86.56%) as brown solid.

### Step C

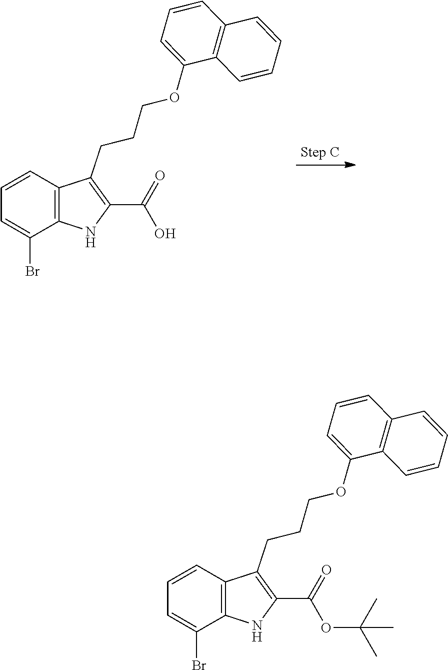 [SMILES: O=C(O)c1[nH]c2c(Br)cccc2c1CCCOc1cccc2ccccc12 ; CC(C)(C)OC(=O)c1[nH]c2c(Br)cccc2c1CCCOc1cccc2ccccc12]

7-Bromo-3-(3-(naphthalen-1-yloxy)propyl)-1H-indole-2-carboxylic acid (3 g, 7.092 mmol) was suspended in toluene (45 mL) and the mixture was heated to reflux under nitrogen. N,N-dimethylformamide di-tert-butyl acetal (4.3 ml, 21.277 mmol) was added drop wise to the refluxing reaction mixture and the reaction was continued for an additional 16 h under nitrogen. After 16 h another 2.8 mL (14.2 mmol) of N,N-dimethylformamide di-tert-butyl acetal was added to it and the reaction was continued for another 8 h. Reaction mixture was then diluted with EtOAc, washed successively with sodium bicarbonate (saturated), water and brine. Organic layer was dried over anhydrous Na₂SO₄ and evaporated under reduced pressure to get the crude compound, which was then purified by column chromatography (SiO₂, DCM:MeOH 2%) to get tert-butyl 7-bromo-3-(3-(naphthalen-1-yloxy)propyl)-1H-indole-2-carboxylate (2 g, 4.17 mmol, 58.7%) as brown solid.

LCMS (ESI−): m/z 480.3 [M−H]⁻

### Step D

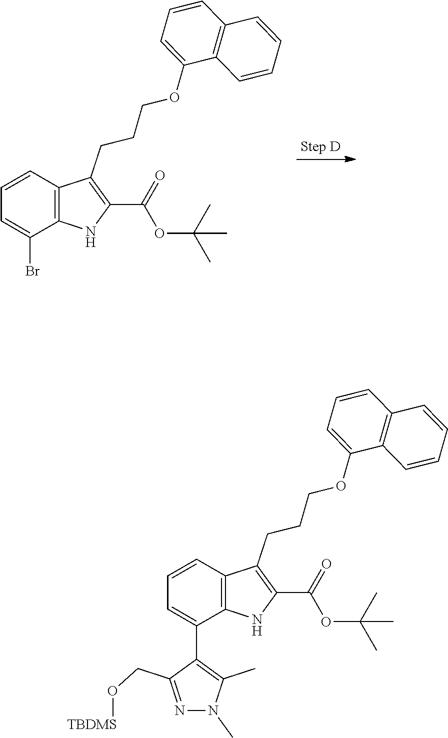

To a solution of tert-butyl 7-bromo-3-(3-(naphthalen-1-yloxy)propyl)-1H-indole-2-carboxylate (1.8 g, 3.758 mmol) in dioxane (30 ml) and water (6 ml) were added 3-(((tert-butyldimethylsilyl)oxy)methyl)-1,5-dimethyl-4-(4,4,5,5-tetramethyl-1,3,2-dioxaborolan-2-yl)-1H-pyrazole (2.75 g, 7.516 mmol) and K₂CO₃ (2 g, 15.031 mmol). The mixture was deoxygenated with argon and to it was added Pd(dppf)Cl₂ (412 mg, 0.564 mmol) under argon atmosphere. Then the reaction mixture was heated under reflux for 16 h. After complete consumption of the starting material (monitored by TLC and LCMS) the reaction mixture was filtered through celite and the solvent was evaporated under reduced pressure get the crude material. It was then diluted with EtOAc, washed successively with water and brine, the organic layer was dried over Na₂SO₄ and evaporated under reduced pressure to get tert-butyl 7-(3-(((tert-butyldimethylsilyl)oxy)methyl)-1,5-dimethyl-1H-pyrazol-4-yl)-3-(3-(naphthalen-1-yloxy)propyl)-1H-indole-2-carboxylate (2 μg, crude) as brown gummy liquid. The crude product was used for the next step without further purification.

LCMS (ESI+): m/z 640.7 [M+H]⁺.

### Step E

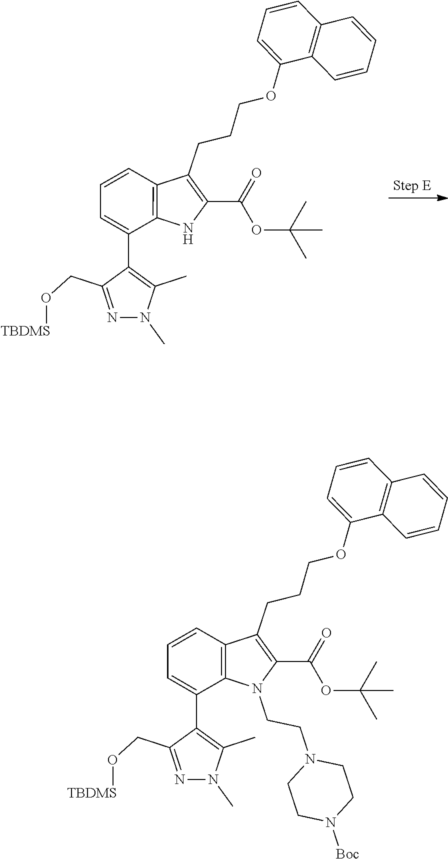

To a solution of tert-butyl 7-(3-(((tert-butyldimethylsilyl)oxy)methyl)-1,5-dimethyl-1H-pyrazol-4-yl)-3-(3-(naphthalen-1-yloxy)propyl)-1H-indole-2-carboxylate (2 g, crude) in DMF (20 ml) was added tert-butyl 4-(2-chloroethyl)piperazine-1-carboxylate (1.6 g, 6.463 mmol) followed by cesium carbonate (4.2 g, 12.926 mmol) in DMF and the mixture was allowed to stir at 90° C. for 16 h under nitrogen. After complete consumption of the starting material (monitored by TLC and LCMS) the reaction mixture was diluted with EtOAc, washed successively with water and brine, the organic layer was dried over sodium sulphate and evaporated under reduced pressure to get the crude compound, which was then purified by column chromatography (SiO₂, 50% EtOAc in DCM) to get tert-butyl 1-(2-(4-(tert-butoxycarbonyl)piperazin-1-yl)ethyl)-7-(3-(((tert-butyldimethylsilyl)oxy)methyl)-1,5-dimethyl-1H-pyrazol-4-yl)-3-(3-(naphthalen-1-yloxy)propyl)-1H-indole-2-carboxylate (1.4 g, 1.64 mmol, 44% over two steps) as gummy solid.

### Step F

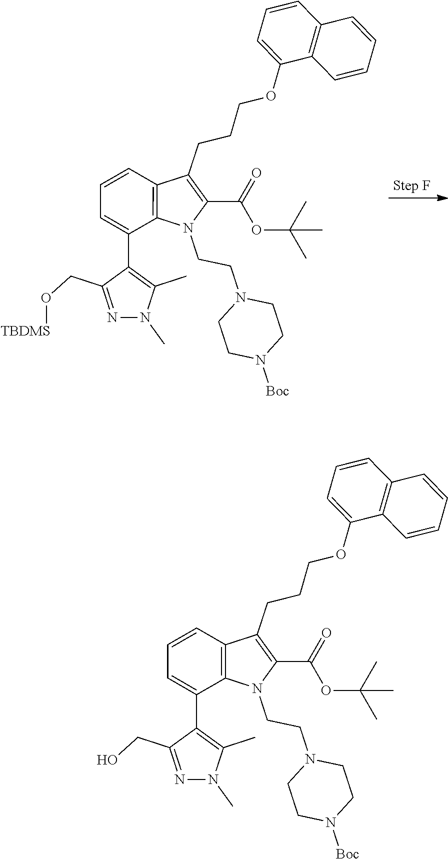

To a stirred solution of tert-butyl 1-(2-(4-(tert-butoxycarbonyl)piperazin-1-yl)ethyl)-7-(3-(((tert-butyldimethylsilyl)oxy)methyl)-1,5-dimethyl-1H-pyrazol-4-yl)-3-(3-(naphthalen-1-yloxy)propyl)-1H-indole-2-carboxylate (1.4 g, 1.64 mmol) in THE (20 mL) was added TBAF (8 mL) drop-wise at 0° C. under nitrogen and the reaction mixture was allowed to stir at ambient temperature for 2 h. After complete consumption of the starting material (monitored by TLC and LCMS) the excess TBAF of the reaction mixture was quenched by addition of an aqueous solution of NaHCO₃. The reaction mixture was then diluted with EtOAc and washed successively with water and brine, the organic layer was dried over sodium sulphate and evaporated under reduced pressure to get the crude compound, which was then purified by column chromatography (SiO₂, 5% MeOH in DCM) to get tert-butyl 1-(2-(4-(tert-butoxycarbonyl)piperazin-1-yl)ethyl)-7-(3-(hydroxymethyl)-1,5-dimethyl-1H-pyrazol-4-yl)-3-(3-(naphthalen-1-yloxy)propyl)-1H-indole-2-carboxylate (500 mg, 0.678 mmol, 42%) as gummy solid.

LCMS (ESI+): m/z 738.5 [M+H]⁺

### Step G

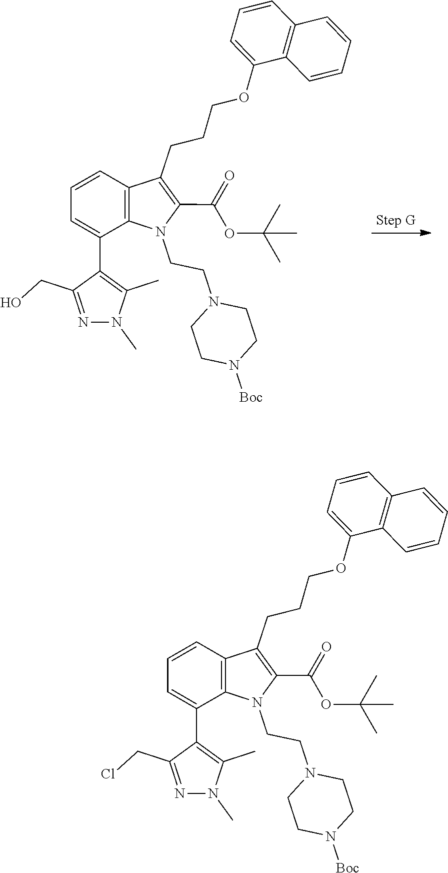 [SMILES: Cc1c(-c2cccc3c(CCCOc4cccc5ccccc45)c(C(=O)OC(C)(C)C)n(CCN4CCN(C(=O)OC(C)(C)C)CC4)c23)c(CO)nn1C ; Cc1c(-c2cccc3c(CCCOc4cccc5ccccc45)c(C(=O)OC(C)(C)C)n(CCN4CCN(C(=O)OC(C)(C)C)CC4)c23)c(CCl)nn1C]

To a solution of tert-butyl 1-(2-(4-(tert-butoxycarbonyl)piperazin-1-yl)ethyl)-7-(3-(hydroxymethyl)-1,5-dimethyl-1H-pyrazol-4-yl)-3-(3-(naphthalen-1-yloxy)propyl)-1H-indole-2-carboxylate (200 mg, 0.271 mmol) in DCM (10 mL) was added thionyl chloride (0.1 mL, 0.543 mmol) drop-wise at 0° C. The reaction mixture was allowed to reach room temperature and was stirred for 1 h. After complete consumption of the starting material (monitored by TLC and LCMS) the reaction mixture was then poured into an ice-cold solution of saturated aqueous NaHCO₃ (20 mL) and DCM (20 mL). The phases were separated and the aqueous phase was re-extracted with DCM. The combined organic phases were washed with water (1×50 ml), dried over MgSO₄ and concentrated under reduced pressure to give 150 mg crude of tert-butyl 1-(2-(4-(tert-butoxycarbonyl)piperazin-1-yl)ethyl)-7-(3-(chloromethyl)-1,5-dimethyl-1H-pyrazol-4-yl)-3-(3-(naphthalen-1-yloxy)propyl)-1H-indole-2-carboxylate which was used for the next step without further purification.

LCMS (ESI+): m/z 756.5 [M+H]⁺

### Step H

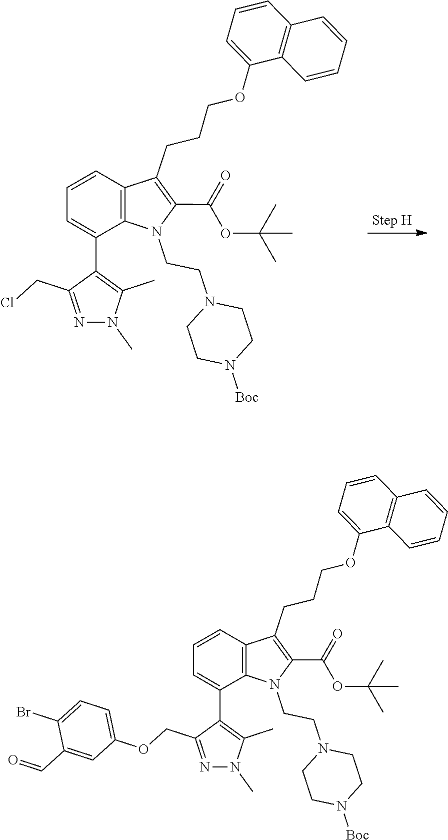 [SMILES: Cc1c(-c2cccc3c(CCCOc4cccc5ccccc45)c(C(=O)OC(C)(C)C)n(CCN4CCN(C(=O)OC(C)(C)C)CC4)c23)c(CCl)nn1C ; Cc1c(-c2cccc3c(CCCOc4cccc5ccccc45)c(C(=O)OC(C)(C)C)n(CCN4CCN(C(=O)OC(C)(C)C)CC4)c23)c(COc2ccc(Br)c(C=O)c2)nn1C]

2-Bromo-5-hydroxybenzaldehyde (60 mg, 0.298 mmol) and potassium tert-butoxide (0.4 ml, 0.397 mmol) were dissolved in DMF (3 mL), followed by the addition of tert-butyl 1-(2-(4-(tert-butoxycarbonyl)piperazin-1-yl)ethyl)-7-(3-(chloromethyl)-1,5-dimethyl-1H-pyrazol-4-yl)-3-(3-(naphthalen-1-yloxy)propyl)-1H-indole-2-carboxylate (150 mg, crude). The resulting mixture was stirred at 60° C. for 2 h under nitrogen. After complete consumption of the starting material (monitored by TLC and LCMS) the reaction mixture was then cooled down to room temperature, diluted with ethyl acetate and washed successively with water and brine. The organic layer was dried over sodium sulphate and evaporated under reduced pressure to get the crude compound, which was then purified by preparative HPLC to afford tert-butyl 7-(3-((4-bromo-3-formylphenoxy)methyl)-1,5-dimethyl-1H-pyrazol-4-yl)-1-(2-(4-(tert-butoxycarbonyl)piperazin-1-yl)ethyl)-3-(3-(naphthalen-1-yloxy)propyl)-1H-indole-2-carboxylate (15 mg, 0.016 mmol, 5% over two steps) as off white solid.

LCMS (ESI+): m/z 920.2 [M+H]⁺

### Step I

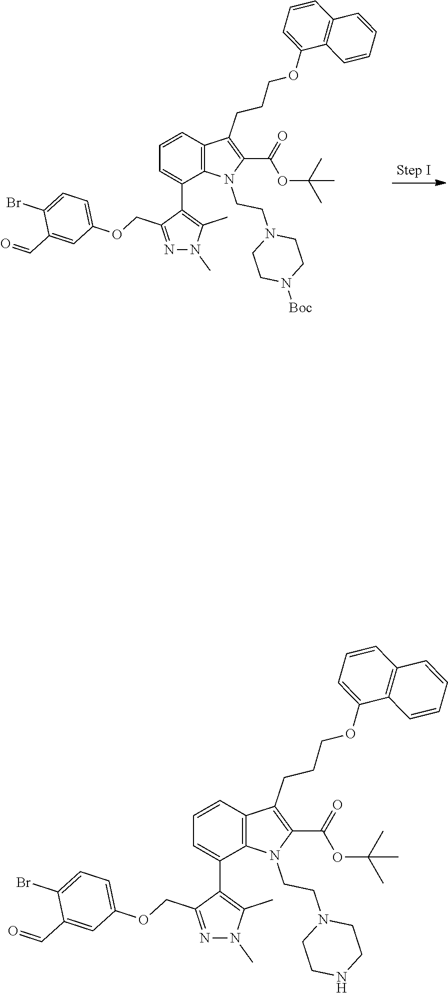 [SMILES: Cc1c(-c2cccc3c(CCCOc4cccc5ccccc45)c(C(=O)OC(C)(C)C)n(CCN4CCN(C(=O)OC(C)(C)C)CC4)c23)c(COc2ccc(Br)c(C=O)c2)nn1C ; Cc1c(-c2cccc3c(CCCOc4cccc5ccccc45)c(C(=O)OC(C)(C)C)n(CCN4CCNCC4)c23)c(COc2ccc(Br)c(C=O)c2)nn1C]

To a stirred solution of tert-butyl 7-(3-((4-bromo-3-formylphenoxy)methyl)-1,5-dimethyl-1H-pyrazol-4-yl)-1-(2-(4-(tert-butoxycarbonyl)piperazin-1-yl)ethyl)-3-(3-(naphthalen-1-yloxy)propyl)-1H-indole-2-carboxylate (200 mg, 0.218 mmol) in DCM (2 mL) was added 2 ml of 4M HCl in dioxan at 0° C. under nitrogen and the reaction mixture was allowed to stir at room temperature for 1 h. After consumption of the starting material (monitored by TLC and LCMS) the reaction mixture was quenched by the slow addition of aq. 1M NaOH at 0° C. to the pH=7. Then it was extracted with DCM (3×50 mL), dried over Na₂SO₄, filtered, and concentrated in vacuo to afford 180 mg of crude tert-butyl 7-(3-((4-bromo-3-formylphenoxy)methyl)-1,5-dimethyl-1H-pyrazol-4-yl)-3-(3-(naphthalen-1-yloxy)propyl)-1-(2-(piperazin-1-yl)ethyl)-1H-indole-2-carboxylate as brown solid.

LCMS (ESI+): m/z 822.8 [M+H]⁺

### Step J

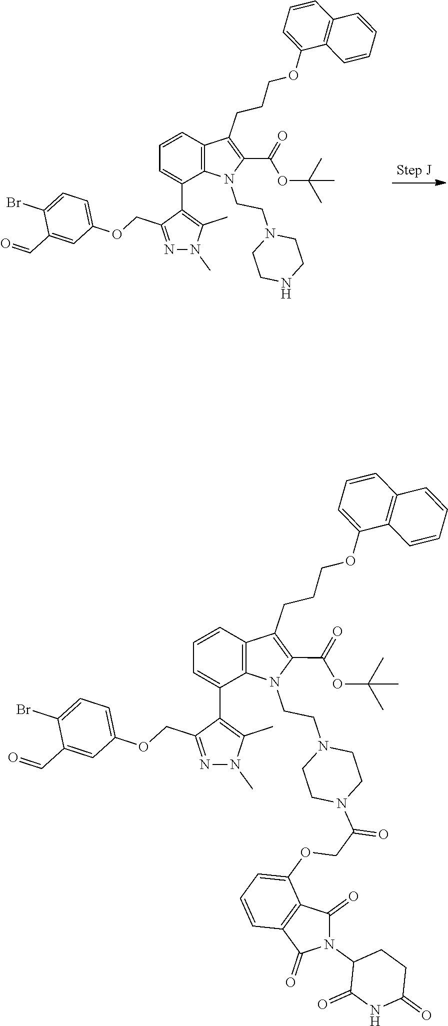

2-((2-(2,6-Dioxopiperidin-3-yl)-1,3-dioxoisoindolin-4-yl)oxy)acetic acid (82 mg, 0.249 mmol) was added to a mixture of tert-butyl 7-(3-((4-bromo-3-formylphenoxy)methyl)-1,5-dimethyl-1H-pyrazol-4-yl)-3-(3-(naphthalen-1-yloxy)propyl)-1-(2-(piperazin-1-yl)ethyl)-1H-indole-2-carboxylate (170 mg, crude) in DMF (3 ml). The mixture was stirred in room temperature under nitrogen atmosphere and to it was added DIPEA (0.2 ml, 0.83 mmol), followed by HATU (236 mg, 0.623 mmol) and the mixture was stirred for additional 16 h. After complete consumption of the starting material (monitored by TLC and LCMS) the reaction mixture diluted with ethyl acetate, washed successively with cold water (3 times) and brine. Organic layer was dried over Na₂SO₄ and evaporated under reduced pressure to get the 180 mg of crude tert-butyl 7-(3-((4-bromo-3-formylphenoxy)methyl)-1,5-dimethyl-1H-pyrazol-4-yl)-1-(2-(4-(2-((2-(2,6-dioxopiperidin-3-yl)-1,3-dioxoisoindolin-4-yl)oxy)acetyl)piperazin-1-yl)ethyl)-3-(3-(naphthalen-1-yloxy)propyl)-1H-indole-2-carboxylate as black solid which was used for next step without further purification.

LCMS (ESI+): m/z 1135.25 [M+H]⁺

### Step K

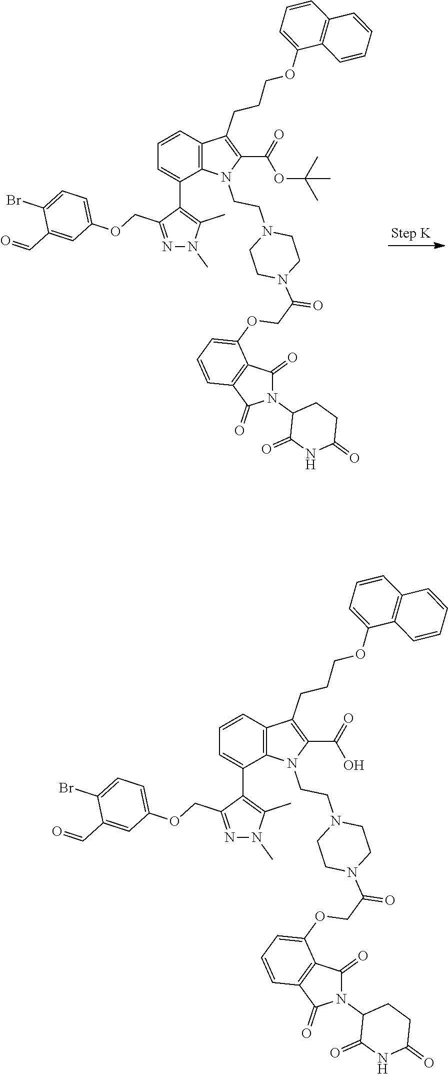

Tert-butyl 7-(3-((4-bromo-3-formylphenoxy)methyl)-1,5-dimethyl-1H-pyrazol-4-yl)-1-(2-(4-(2-((2-(2,6-dioxopiperidin-3-yl)-1,3-dioxoisoindolin-4-yl)oxy)acetyl)piperazin-1-yl)ethyl)-3-(3-(naphthalen-1-yloxy)propyl)-1H-indole-2-carboxylate (180 mg, crude) was suspended in 1,4-dioxane (1.0 mL) and the reaction mixture was cooled to 0° C. then added 2 ml of 4M HCl in dioxane drop wise to the reaction mixture, then the reaction mixture was allowed to stir at ambient temperature for 16 h under nitrogen. After complete consumption of the starting material (monitored by TLC and LCMS) the volatiles were evaporated under reduced pressure to afford the crude reaction mass which was purified by reverse phase preparatory HPLC (H₂O:MeCN+0.1% FA) to afford 7-(3-((4-bromo-3-formylphenoxy)methyl)-1,5-dimethyl-1H-pyrazol-4-yl)-1-(2-(4-(2-((2-(2,6-dioxopiperidin-3-yl)-1,3-dioxoisoindolin-4-yl)oxy)acetyl)piperazin-1-yl)ethyl)-3-(3-(naphthalen-1-yloxy)propyl)-1H-indole-2-carboxylic acid (5.5 mg, 0.0051 mmol, 2.3% over three steps) as white solid.

LCMS (ESI+): m/z 1078.7 [M+H]⁺

¹H NMR (400 MHz, DMSO) δ 11.09 (s, 1H), 10.06 (s, 1H), 8.28-8.17 (m, 1H), 7.90-7.80 (m, 1H), 7.78-7.70 (m, 1H), 7.67 (d, J=7.7 Hz, 1H), 7.56-7.47 (m, 3H), 7.47-7.40 (m, 2H), 7.35 (t, J=7.9 Hz, 1H), 7.28 (d, J=8.6 Hz, 1H), 7.21 (d, J=3.2 Hz, 1H), 7.09-6.98 (m, 2H), 6.96 (d, J=6.8 Hz, 1H), 6.89-6.80 (m, 1H), 5.15-5.04 (m, 3H), 4.98-4.84 (m, 2H), 4.43-4.25 (m, 2H), 4.24-4.10 (m, 2H), 3.83 (s, 3H), 3.29 (s, 6H), 2.95-2.80 (m, 1H), 2.61 (s, 2H), 2.24-2.15 (m, 2H), 2.10 (s, 6H), 2.05-1.96 (m, 4H).

### Example 44. 7-(4-((5-((2-(2,6-dioxopiperidin-3-yl)-1,3-dioxoisoindolin-4-yl)amino)pentyl)carbamoyl)-2,6-dimethylphenyl)-3-(3-(naphthalen-1-yloxy)propyl)pyrazolo[1,5-a]pyridine-2-carboxylic acid (253)

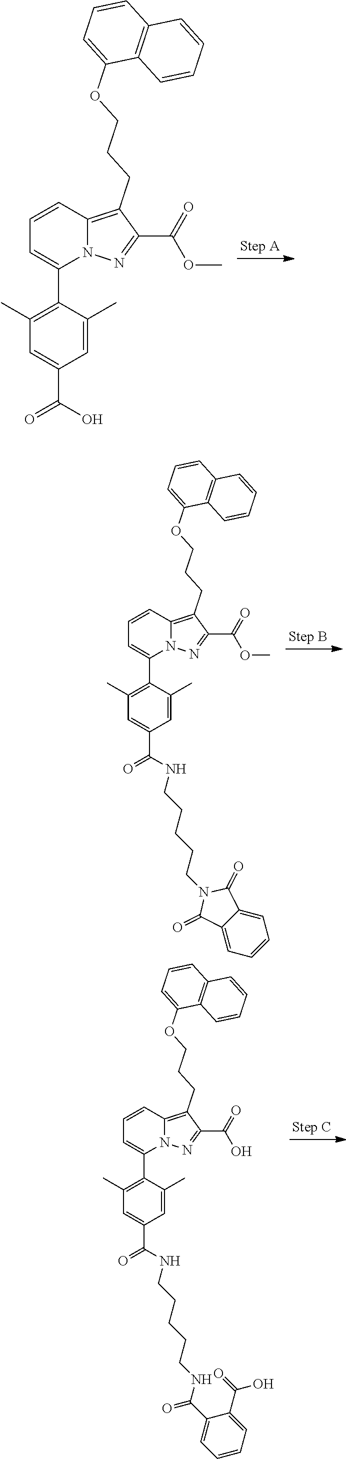

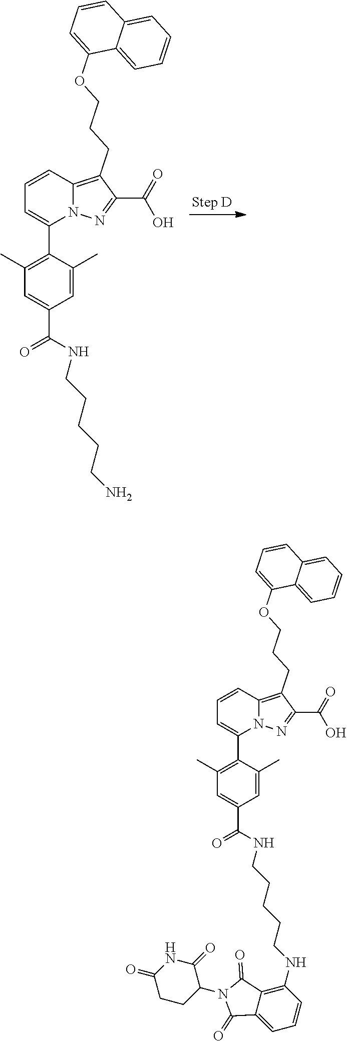 [SMILES: Cc1cc(C(=O)NCCCCCN)cc(C)c1-c1cccc2c(CCCOc3cccc4ccccc34)c(C(=O)O)nn12 ; Cc1cc(C(=O)NCCCCCNc2cccc3c2C(=O)N(C2CCC(=O)NC2=O)C3=O)cc(C)c1-c1cccc2c(CCCOc3cccc4ccccc34)c(C(=O)O)nn12]

### Step A

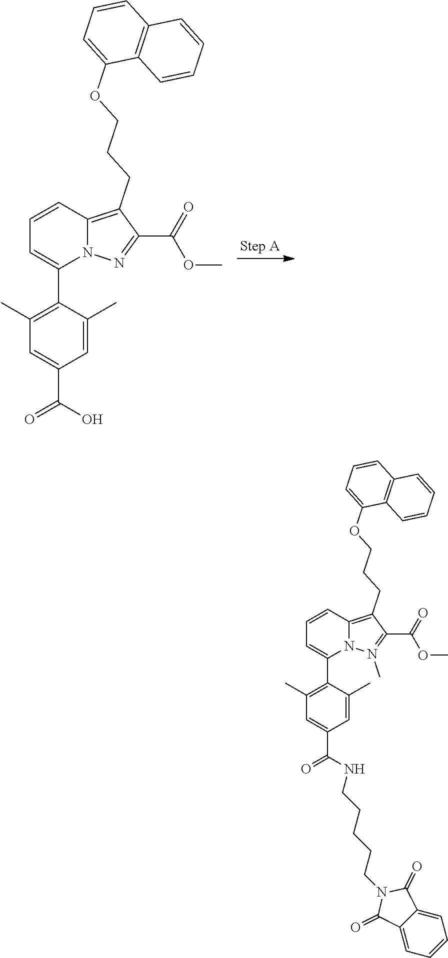

4-(2-(Methoxycarbonyl)-3-(3-(naphthalen-1-yloxy)propyl)pyrazolo[1,5-a]pyridin-7-yl)-3,5-dimethylbenzoic acid (100 mg, 0.20 mmol) was dissolved in anhydrous DMF (1.26 mL). DIPEA (103 L, 0.59 mmol) and HATU (90 mg, 0.24 mmol) were added under inert atmosphere, and mixture was stirred in RT for 1 h. Then 2-(5-(12-azaneyl)pentyl)isoindoline-1,3-dione (55 mg, 0.24 mmol) was added in solution of DMF (1.26 mL). Vial was sealed, and mixture was stirred at RT for overnight. Solvent was removed under reduced pressure, brine and DCM were added and product was extracted with DCM to give methyl 7-(4-((5-(1,3-dioxoisoindolin-2-yl)pentyl)carbamoyl)-2,6-dimethylphenyl)-3-(3-(naphthalen-1-yloxy)propyl)pyrazolo[1,5-a]pyridine-2-carboxylate (142.0 mg of crude) which was used without further purification to the next step.

LCMS (ESI+): m/z 723.2 [M+H]⁺

### Step B

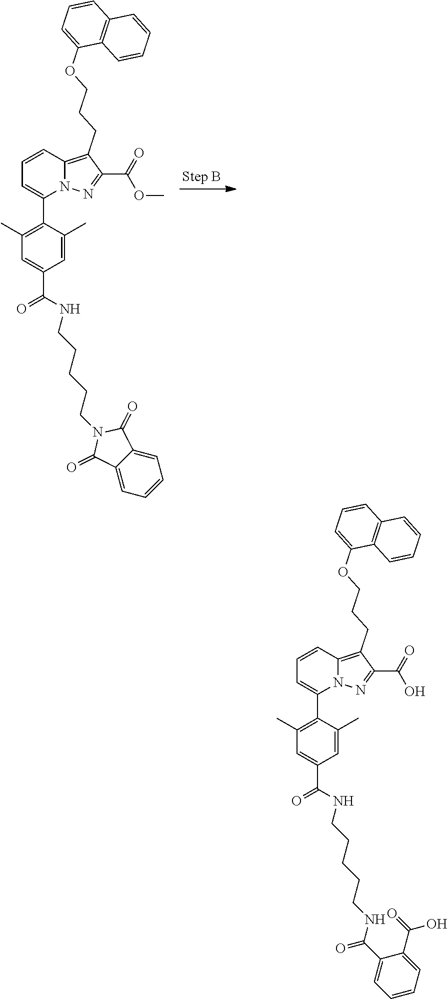

To solution of methyl 7-(4-((5-(1,3-dioxoisoindolin-2-yl)pentyl)carbamoyl)-2,6-dimethylphenyl)-3-(3-(naphthalen-1-yloxy)propyl)pyrazolo[1,5-a]pyridine-2-carboxylate (145 mg of crude) in a mixture MeOH:H₂O:THF 1:1:1 (2.16 mL) was added NaOH dissolved in 0.5 mL of H₂O. Mixture was stirred at 70° C. for 18 h. Solvents were removed under reduced pressure, mixture was extracted with DCM, then aqueous phase was acidified until pH=1 and product was extracted with DCM:MeOH 9:1 to give 7-(4-((5-(2-carboxybenzamido)pentyl)carbamoyl)-2,6-dimethylphenyl)-3-(3-(naphthalen-1-yloxy)propyl)pyrazolo[1,5-a]pyridine-2-carboxylic acid (123 mg, 0.174 mmol, 87% over two steps) as orange foam.

LCMS (ESI+): m/z 727.4 [M+H]⁺

### Step C

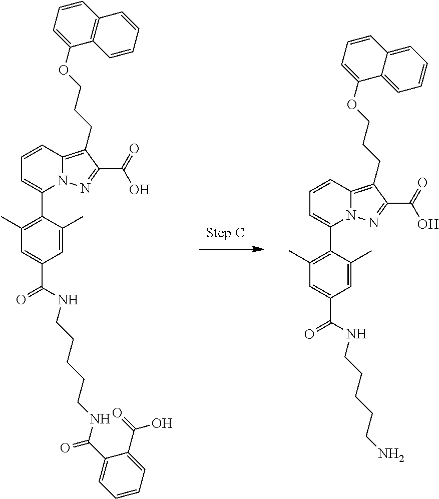 [SMILES: Cc1cc(C(=O)NCCCCCNC(=O)c2ccccc2C(=O)O)cc(C)c1-c1cccc2c(CCCOc3cccc4ccccc34)c(C(=O)O)nn12 ; Cc1cc(C(=O)NCCCCCN)cc(C)c1-c1cccc2c(CCCOc3cccc4ccccc34)c(C(=O)O)nn12]

Hydrazine hydrate 50% (30 μL, 0.47 mmol) was added to solution of 7-(4-((5-(2-carboxybenzamido)pentyl)carbamoyl)-2,6-dimethylphenyl)-3-(3-(naphthalen-1-yloxy)propyl)pyrazolo[1,5-a]pyridine-2-carboxylic acid (115 mg, 0.16 mmol) in MeOH (0.63 mL) and mixture was heated in 60° C. for 18 h. Precipitate was observed which was filtered off. 7-(4-((5-aminopentyl)carbamoyl)-2,6-dimethylphenyl)-3-(3-(naphthalen-1-yloxy)propyl)pyrazolo[1,5-a]pyridine-2-carboxylic acid (45.00 mg, 0.078 mmol, 60%) was obtained as white solid.

LCMS (ESI+): m/z 579.3 [M+H]⁺

### Step D

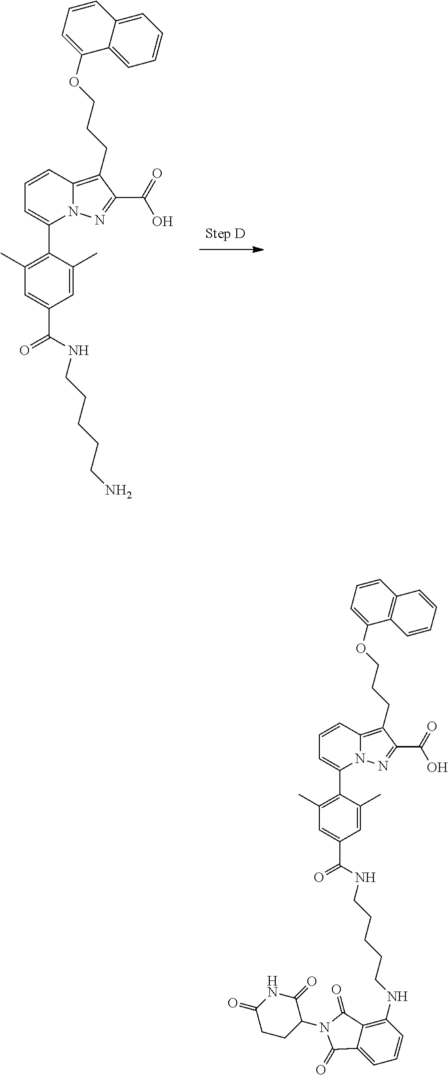 [SMILES: Cc1cc(C(=O)NCCCCCN)cc(C)c1-c1cccc2c(CCCOc3cccc4ccccc34)c(C(=O)O)nn12 ; Cc1cc(C(=O)NCCCCCNc2cccc3c2C(=O)N(C2CCC(=O)NC2=O)C3=O)cc(C)c1-c1cccc2c(CCCOc3cccc4ccccc34)c(C(=O)O)nn12]

DIPEA (18 μL, 0.10 mmol) was added to a solution of 7-(4-((5-aminopentyl)carbamoyl)-2,6-dimethylphenyl)-3-(3-(naphthalen-1-yloxy)propyl)pyrazolo[1,5-a]pyridine-2-carboxylic acid (20 mg, 0.03 mmol) and 2-(2,6-dioxopiperidin-3-yl)-4-fluoroisoindoline-1,3-dione (29 mg, 0.10 mmol) in DMSO (0.24 mL). The reaction mixture was heated at 90° C. for 20 h. Crude was purified by flash chromatography (SiO₂, 5% MeOH in DCM). 7-(4-((5-((2-(2,6-Dioxopiperidin-3-yl)-1,3-dioxoisoindolin-4-yl)amino)pentyl)carbamoyl)-2,6-dimethylphenyl)-3-(3-(naphthalen-1-yloxy)propyl)pyrazolo[1,5-a]pyridine-2-carboxylic acid (7.50 mg, 0.009 mmol, 30%) was isolated as yellow solid.

LCMS (ESI+): m/z 835.2 [M+H]⁺

¹H NMR (600 MHz, DMSO) δ 12.99 (s, 1H), 11.08 (s, 1H), 8.48 (t, J=5.7 Hz, 1H), 8.25 (dd, J=8.2, 1.7 Hz, 1H), 7.90-7.85 (m, 1H), 7.85-7.76 (m, 1H), 7.64 (s, 2H), 7.58 (dd, J=8.5, 7.1 Hz, 1H), 7.56-7.48 (m, 2H), 7.45 (d, J=8.3 Hz, 1H), 7.38 (t, J=7.9 Hz, 1H), 7.29-7.20 (m, 1H), 7.12 (d, J=8.6 Hz, 1H), 7.02 (d, J=7.0 Hz, 1H), 6.89 (d, J=7.5 Hz, 2H), 6.56 (t, J=5.9 Hz, 1H), 5.04 (dd, J=12.8, 5.5 Hz, 1H), 4.17 (t, J=6.2 Hz, 2H), 3.30-3.27 (m, 3H), 2.87 (ddd, J=16.9, 13.8, 5.5 Hz, 1H), 2.60-2.57 (m, 1H), 2.59-2.54 (m, 1H), 2.54-2.50 (m, 1H), 2.47-2.41 (m, 1H), 2.26-2.19 (m, 2H), 2.05-1.99 (m, 1H), 1.94 (s, 7H), 1.66-1.57 (m, 4H), 1.42 (t, J=7.6 Hz, 2H).

### Example 45. 7-(4-((2-(2-((2-(2,6-dioxopiperidin-3-yl)-1,3-dioxoisoindolin-4-yl)amino)ethoxy)ethyl)carbamoyl)-2,6-dimethylphenyl)-3-(3-(naphthalen-1-yloxy)propyl)pyrazolo[1,5-a]pyridine-2-carboxylic acid (254)

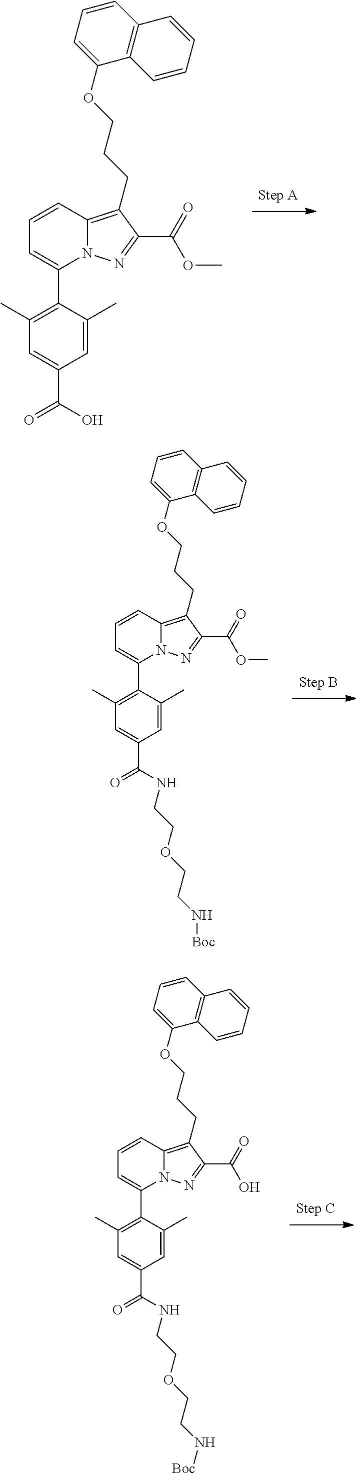

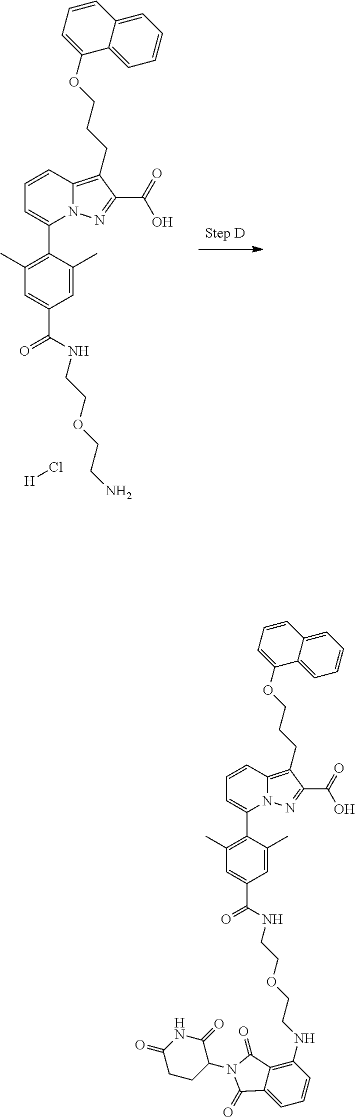 [SMILES: Cc1cc(C(=O)NCCOCCN)cc(C)c1-c1cccc2c(CCCOc3cccc4ccccc34)c(C(=O)O)nn12 ; Cl ; Cc1cc(C(=O)NCCOCCNc2cccc3c2C(=O)N(C2CCC(=O)NC2=O)C3=O)cc(C)c1-c1cccc2c(CCCOc3cccc4ccccc34)c(C(=O)O)nn12]

### Step A

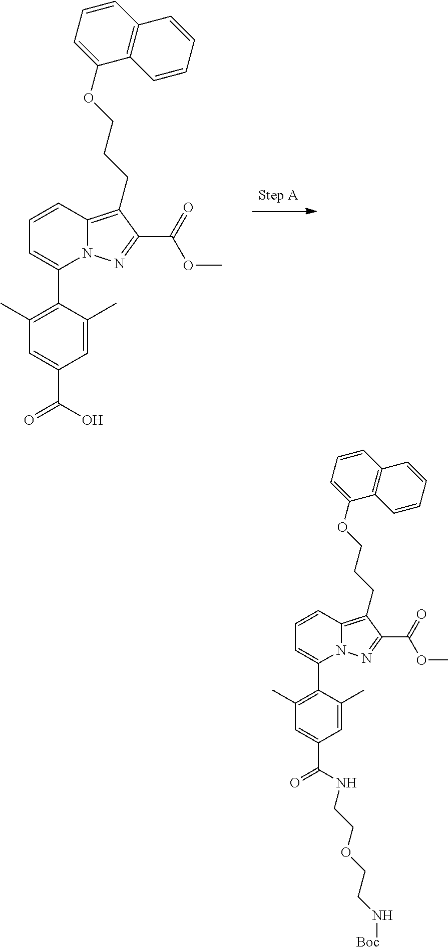

4-(2-(Methoxycarbonyl)-3-(3-(naphthalen-1-yloxy)propyl)pyrazolo[1,5-a]pyridin-7-yl)-3,5-dimethylbenzoic acid (100 mg, 0.20 mmol) and HATU (90 mg, 0.24 mmol) were dissolved in anhydrous DMF (1.5 mL) and DIPEA (76 mg, 0.59 mmol) was added. The solution was stirred in an inert atmosphere at RT for 1 h. In a separate vial tert-butyl (2-(2-aminoethoxy)ethyl)carbamate (48 mg, 0.24 mmol) was dissolved in anhydrous DMF (0.5 mL) and added to the main solution. The mixture was stirred at RT overnight. DMF was evaporated and the residue was dissolved in DCM and brine was added followed by extraction by DCM. The solvent was removed on rotavapor and the crude methyl 7-(4-((2-(2-((tert-butoxycarbonyl)amino)ethoxy)ethyl)carbamoyl)-2,6-dimethylphenyl)-3-(3-(naphthalen-1-yloxy)propyl)pyrazolo[1,5-a]pyridine-2-carboxylate (100.0 mg, crude) was used in the next step.

LCMS (ESI+): m/z 595.5 [M+H−Boc]⁺

### Step B

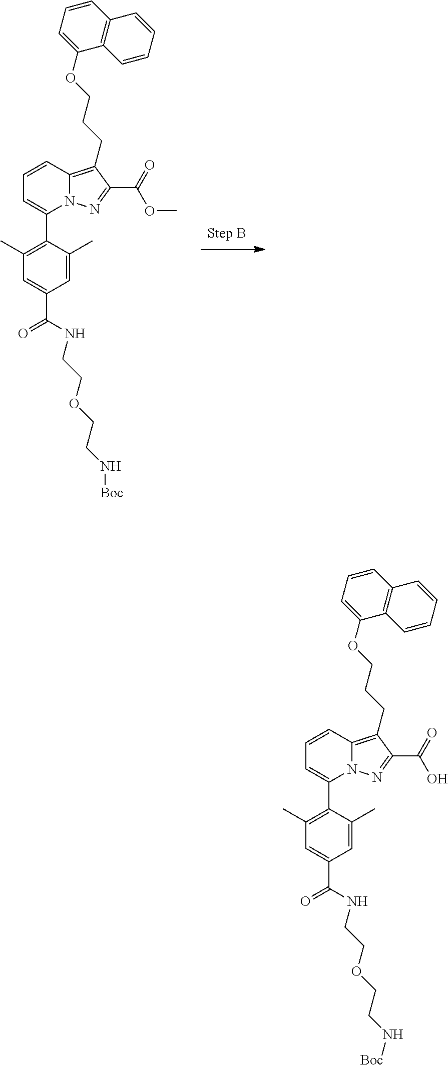

Methyl 7-(4-((2-(2-((tert-butoxycarbonyl)amino)ethoxy)ethyl)carbamoyl)-2,6-dimethylphenyl)-3-(3-(naphthalen-1-yloxy)propyl)pyrazolo[1,5-a]pyridine-2-carboxylate (100 mg, crude) was dissolved in THE (6 mL) and the solution of NaOH (90 mg, 2.25 mmol) in 2 mL of water was added. The solution was stirred at 50° C. for 18 h. The reaction was cooled to RT, water was added and extracted 4 times by EtOAc. The organic phase was evaporated to dryness and the crude 7-(4-((2-(2-((tert-butoxycarbonyl)amino)ethoxy)ethyl)carbamoyl)-2,6-dimethylphenyl)-3-(3-(naphthalen-1-yloxy)propyl)pyrazolo[1,5-a]pyridine-2-carboxylic acid (100.0 mg, crude) was used directly for the next step.

LCMS (ESI+): m/z 581.5 [M+H−Boc]⁺

### Step C

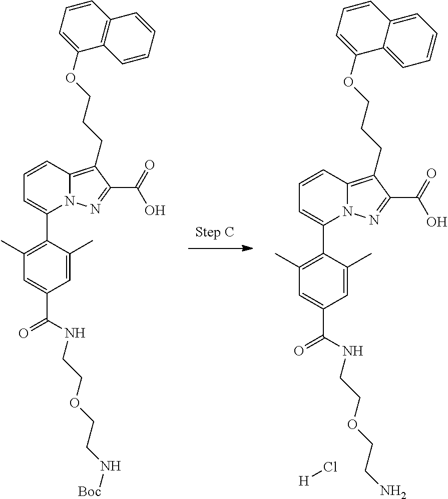

7-(4-((2-(2-((tert-butoxycarbonyl)amino)ethoxy)ethyl)carbamoyl)-2,6-dimethylphenyl)-3-(3-(naphthalen-1-yloxy)propyl)pyrazolo[1,5-a]pyridine-2-carboxylic acid (100 mg, crude) was dissolved in dry THE (1.6 mL) and 4M HCl in dioxane (0.75 mL) was added. The mixture was stirred overnight at RT. Solvents were evaporated to give 7-(4-((2-(2-aminoethoxy)ethyl)carbamoyl)-2,6-dimethylphenyl)-3-(3-(naphthalen-1-yloxy)propyl)pyrazolo[1,5-a]pyridine-2-carboxylic acid hydrochloride (40.0 mg, crude) as yellow sticky gum. The obtained crude was used directly for the next step.

LCMS (ESI+): m/z 581.5 [M+H]⁺

### Step D

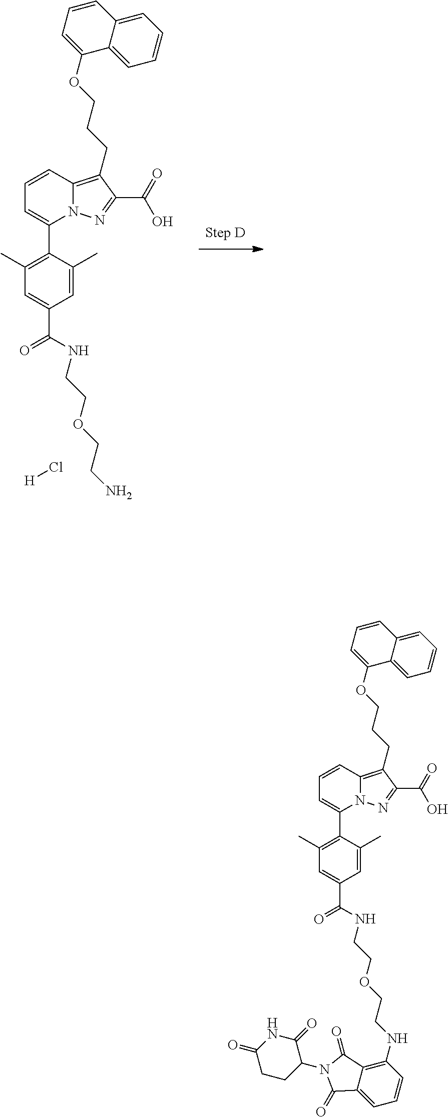 [SMILES: Cc1cc(C(=O)NCCOCCN)cc(C)c1-c1cccc2c(CCCOc3cccc4ccccc34)c(C(=O)O)nn12 ; Cl ; Cc1cc(C(=O)NCCOCCNc2cccc3c2C(=O)N(C2CCC(=O)NC2=O)C3=O)cc(C)c1-c1cccc2c(CCCOc3cccc4ccccc34)c(C(=O)O)nn12]

7-(4-((2-(2-aminoethoxy)ethyl)carbamoyl)-2,6-dimethylphenyl)-3-(3-(naphthalen-1-yloxy)propyl)pyrazolo[1,5-a]pyridine-2-carboxylic acid hydrochloride (9.0 mg, crude) and 2-(2,6-dioxopiperidin-3-yl)-4-fluoroisoindoline-1,3-dione (41 mg, 0.15 mmol) were dissolved in DMSO (0.21 mL) and DIPEA (6 mg, 0.05 mmol) was added to the mixture. Reaction was stirred in 90° C. for 20 h. The solvent was evaporated and the residue was purified by flash chromatography (SiO₂, 5% MeOH in DCM) to obtain 7-(4-((2-(2-((2-(2,6-dioxopiperidin-3-yl)-1,3-dioxoisoindolin-4-yl)amino)ethoxy)ethyl)carbamoyl)-2,6-dimethylphenyl)-3-(3-(naphthalen-1-yloxy)propyl)pyrazolo[1,5-a]pyridine-2-carboxylic acid (2.10 mg, 0.003 mmol, 6% over 4 steps) as yellow solid.

LCMS (ESI+): m/z 837.6 [M+H]⁺

1H NMR (500 MHz, CDCl₃) δ 8.34-8.27 (m, 1H), 7.78-7.70 (m, 1H), 7.53-7.36 (m, 5H), 7.33 (d, J=8.2 Hz, 1H), 7.29-7.20 (m, 1H), 7.04-6.96 (m, 2H), 6.86 (d, J=8.5 Hz, 1H), 6.67 (d, J=7.6 Hz, 1H), 6.65-6.57 (m, 2H), 4.80-4.69 (m, 1H), 4.15-4.02 (m, 2H), 3.75-3.46 (m, 7H), 3.45-3.36 (m, 2H), 3.36-3.26 (m, 2H), 2.73-2.56 (m, 2H), 2.36-2.25 (m, 2H), 1.99-1.80 (m, 7H).

### Example 46. 7-(4-((2-(2-(2-((2-(2,6-dioxopiperidin-3-yl)-1,3-dioxoisoindolin-4-yl)amino)ethoxy)ethoxy)ethyl)carbamoyl)-2,6-dimethylphenyl)-3-(3-(naphthalen-1-yloxy)propyl)pyrazolo[1,5-a]pyridine-2-carboxylic acid (255)

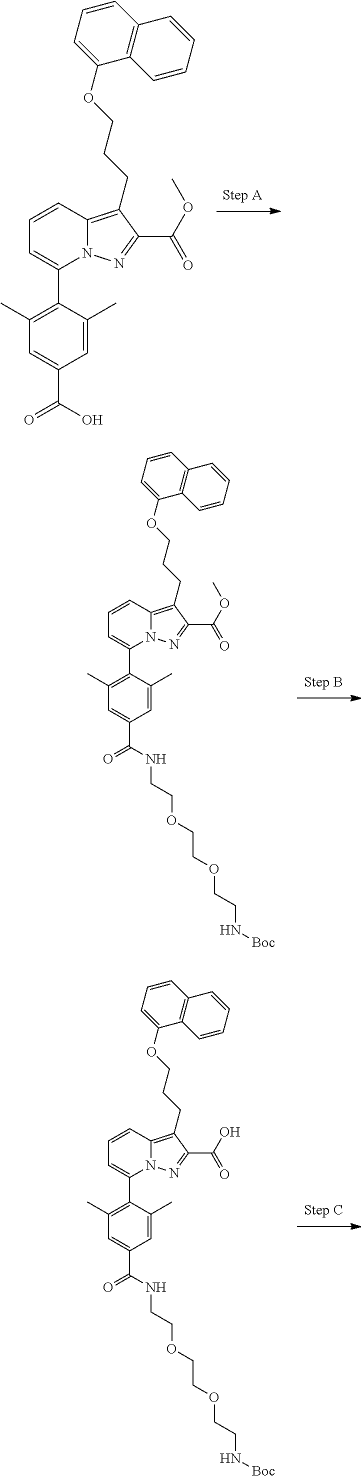 [SMILES: COC(=O)c1nn2c(-c3c(C)cc(C(=O)O)cc3C)cccc2c1CCCOc1cccc2ccccc12 ; COC(=O)c1nn2c(-c3c(C)cc(C(=O)NCCOCCOCCNC(=O)OC(C)(C)C)cc3C)cccc2c1CCCOc1cccc2ccccc12 ; Cc1cc(C(=O)NCCOCCOCCNC(=O)OC(C)(C)C)cc(C)c1-c1cccc2c(CCCOc3cccc4ccccc34)c(C(=O)O)nn12]

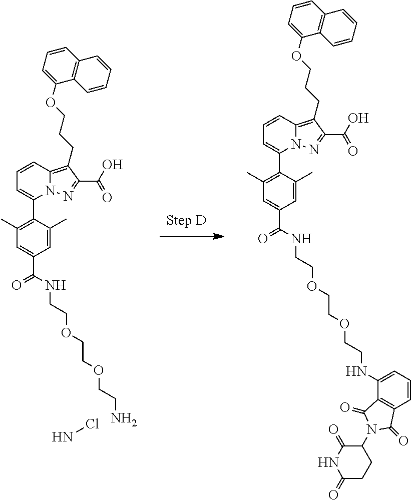

### Step A

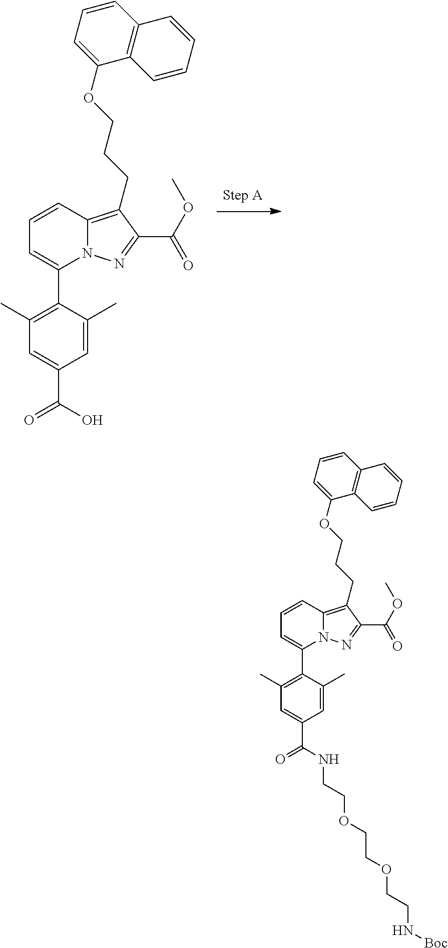 [SMILES: COC(=O)c1nn2c(-c3c(C)cc(C(=O)O)cc3C)cccc2c1CCCOc1cccc2ccccc12 ; COC(=O)c1nn2c(-c3c(C)cc(C(=O)NCCOCCOCCNC(=O)OC(C)(C)C)cc3C)cccc2c1CCCOc1cccc2ccccc12]

4-(2-(methoxycarbonyl)-3-(3-(naphthalen-1-yloxy)propyl)pyrazolo[1,5-a]pyridin-7-yl)-3,5-dimethylbenzoic acid (100 mg, 0.20 mmol) and HATU (90 mg, 0.24 mmol) was dissolved in anhydrous DMF (1.5 mL) and DIPEA (103 μL, 0.59 mmol) was added to the mixture. The reaction was stirred in an inert atmosphere at RT for 1 h. The solution of tert-butyl (2-(2-(2-aminoethoxy)ethoxy)ethyl)carbamate (59 mg, 0.24 mmol) in anhydrous DMF (0.5 mL) was added and the resulting solution was stirred at RT overnight. DMF was evaporated and the residue was taken up in DCM and brine was added followed by extraction by DCM. The solvent was concentrated in vacuo and the crude product of methyl 7-(4-((2,2-dimethyl-4-oxo-3,8,11-trioxa-5-azatridecan-13-yl)carbamoyl)-2,6-dimethylphenyl)-3-(3-(naphthalen-1-yloxy)propyl)pyrazolo[1,5-a]pyridine-2-carboxylate (105 mg, crude) was used without purification in the next step.

LCMS (ESI+): m/z 639.6 [M−Boc+H]⁺

### Step B

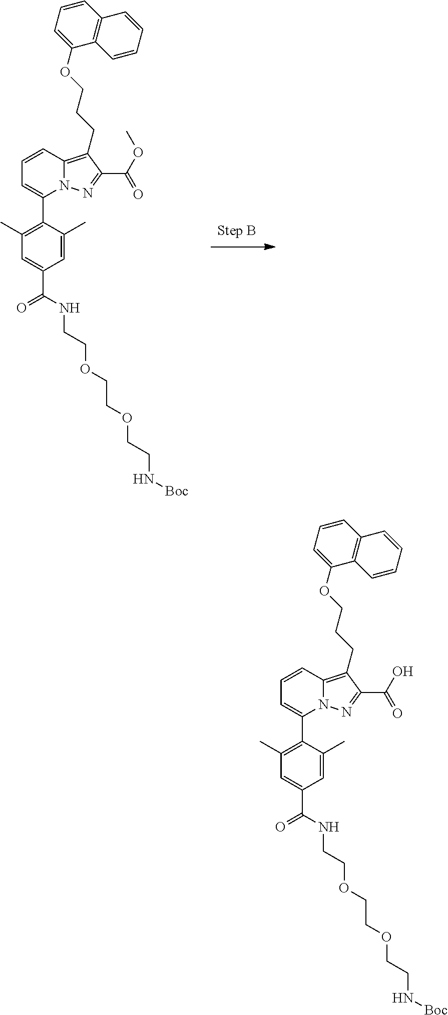 [SMILES: COC(=O)c1nn2c(-c3c(C)cc(C(=O)NCCOCCOCCNC(=O)OC(C)(C)C)cc3C)cccc2c1CCCOc1cccc2ccccc12 ; Cc1cc(C(=O)NCCOCCOCCNC(=O)OC(C)(C)C)cc(C)c1-c1cccc2c(CCCOc3cccc4ccccc34)c(C(=O)O)nn12]

The residue of methyl 7-(4-((2,2-dimethyl-4-oxo-3,8,11-trioxa-5-azatridecan-13-yl)carbamoyl)-2,6-dimethylphenyl)-3-(3-(naphthalen-1-yloxy)propyl)pyrazolo[1,5-a]pyridine-2-carboxylate (105 mg, of crude) was dissolved in THE (6 mL) and the solution of NaOH (90 mg, 2.25 mmol) in 2 mL of H₂O was added. The solution was stirred at 50° C. for 18 h. The reaction was cooled to RT, water was added and the mixture was extracted with EtOAc. The organic phase was evaporated and to give 143 mg crude of 7-(4-((2,2-dimethyl-4-oxo-3,8,11-trioxa-5-azatridecan-13-yl)carbamoyl)-2,6-dimethylphenyl)-3-(3-(naphthalen-1-yloxy)propyl)pyrazolo[1,5-a]pyridine-2-carboxylic acid was used directly for the next step.

LCMS (ESI+): m/z 723.7 [M−H]⁻

### Step C

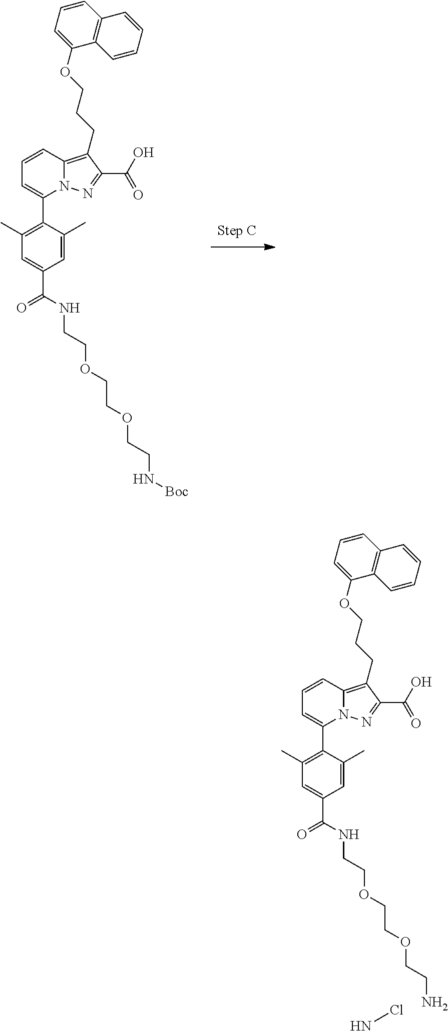

The residue of 7-(4-((2,2-dimethyl-4-oxo-3,8,11-trioxa-5-azatridecan-13-yl)carbamoyl)-2,6-dimethylphenyl)-3-(3-(naphthalen-1-yloxy)propyl)pyrazolo[1,5-a]pyridine-2-carboxylic acid (143 mg, crude) was dissolved in dry THE (2 mL) and 4M HCl in dioxane (344 μL, 1.38 mmol) was added. The resulting solution was mixed overnight at RT. After this time additional portion of 4M HCl in dioxane (885 μL, 3.54 mmol) was added and left stirring overnight. The solvents were evaporated to give 7-(4-((2-(2-(2-aminoethoxy)ethoxy)ethyl)carbamoyl)-2,6-dimethylphenyl)-3-(3-(naphthalen-1-yloxy)propyl)pyrazolo[1,5-a]pyridine-2-carboxylic acid hydrochloride (74.0 mg, crude) which was used directly for the next step.

LCMS (ESI+): m/z 625.6 [M+H]⁺

### Step D

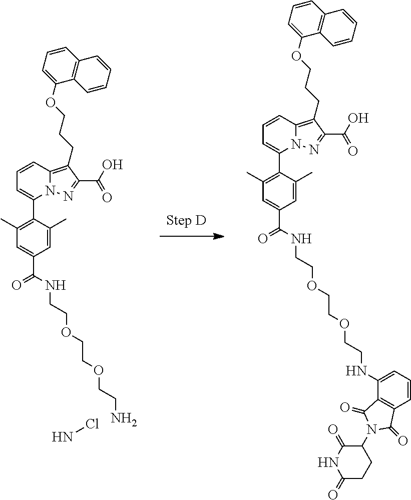

To 7-(4-((2-(2-(2-aminoethoxy)ethoxy)ethyl)carbamoyl)-2,6-dimethylphenyl)-3-(3-(naphthalen-1-yloxy)propyl)pyrazolo[1,5-a]pyridine-2-carboxylic acid hydrochloride (74 mg, crude) and 2-(2,6-dioxopiperidin-3-yl)-4-fluoroisoindoline-1,3-dione (266 mg, 0.96 mmol) suspended in DMSO was added DIPEA (55 uL, 0.29 mmol). The mixture was stirred at 90° C. for 18 h. Then the reaction was cooled, the solvents were evaporated. The residue was purified using flash chromatography (SiO₂, 5% MeOH in DCM) and by preparative TLC (SiO₂, 5% MeOH in DCM). 7-(4-((2-(2-(2-((2-(2,6-dioxopiperidin-3-yl)-1,3-dioxoisoindolin-4-yl)amino)ethoxy)ethoxy)ethyl)carbamoyl)-2,6-dimethylphenyl)-3-(3-(naphthalen-1-yloxy)propyl)pyrazolo[1,5-a]pyridine-2-carboxylic acid (8.50 mg, 0.010 mmol, 10%) was obtained as yellow solid.

LCMS (ESI+): m/z 881.6 [M+H]⁺

1H NMR (500 MHz, CDCl₃) δ 8.34-8.28 (m, 1H), 7.77-7.70 (m, 1H), 7.59 (s, 1H), 7.53-7.46 (m, 2H), 7.46-7.36 (m, 3H), 7.33 (d, J=8.3 Hz, 1H), 7.28-7.23 (m, 1H), 7.03-6.94 (m, 2H), 6.79 (d, J=8.5 Hz, 1H), 6.67 (d, J=7.7 Hz, 1H), 6.65-6.56 (m, 1H), 4.72-4.63 (m, 1H), 4.15-4.04 (m, 2H), 3.74-3.49 (m, 11H), 3.41-3.36 (m, 2H), 3.33 (t, J=7.2 Hz, 2H), 2.71-2.50 (m, 3H), 2.36-2.26 (m, 2H), 1.98-1.83 (m, 6H).

### Example 47. 7-(4-((2-(2-(2-(2-((2-(2,6-dioxopiperidin-3-yl)-1,3-dioxoisoindolin-4-yl)amino)ethoxy)ethoxy)ethoxy)ethyl)carbamoyl)-2,6-dimethylphenyl)-3-(3-(naphthalen-1-yloxy)propyl)pyrazolo[1,5-a]pyridine-2-carboxylic acid (256)

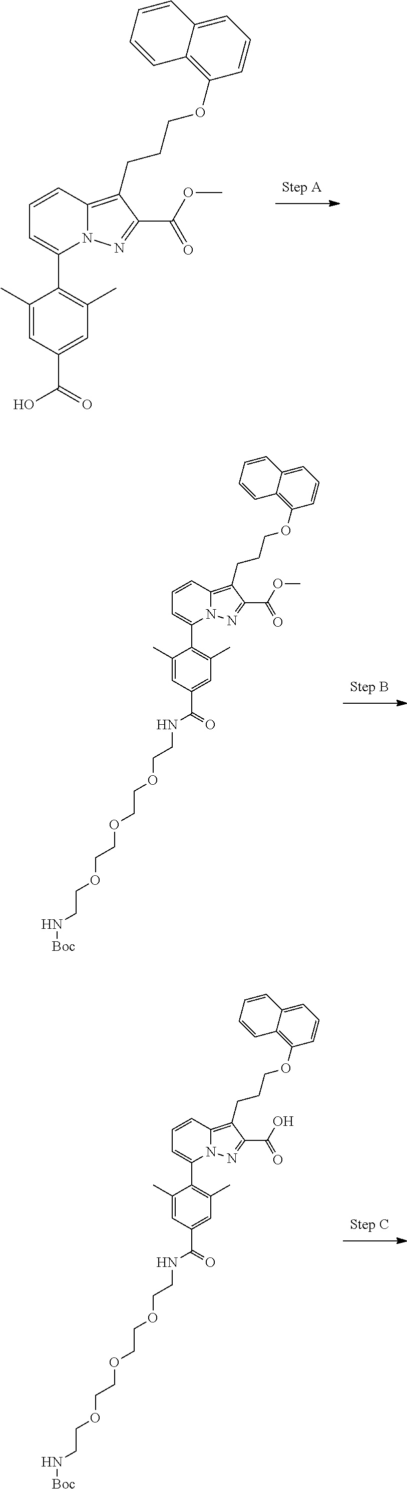 [SMILES: COC(=O)c1nn2c(-c3c(C)cc(C(=O)O)cc3C)cccc2c1CCCOc1cccc2ccccc12 ; COC(=O)c1nn2c(-c3c(C)cc(C(=O)NCCOCCOCCOCCNC(=O)OC(C)(C)C)cc3C)cccc2c1CCCOc1cccc2ccccc12 ; Cc1cc(C(=O)NCCOCCOCCOCCNC(=O)OC(C)(C)C)cc(C)c1-c1cccc2c(CCCOc3cccc4ccccc34)c(C(=O)O)nn12]

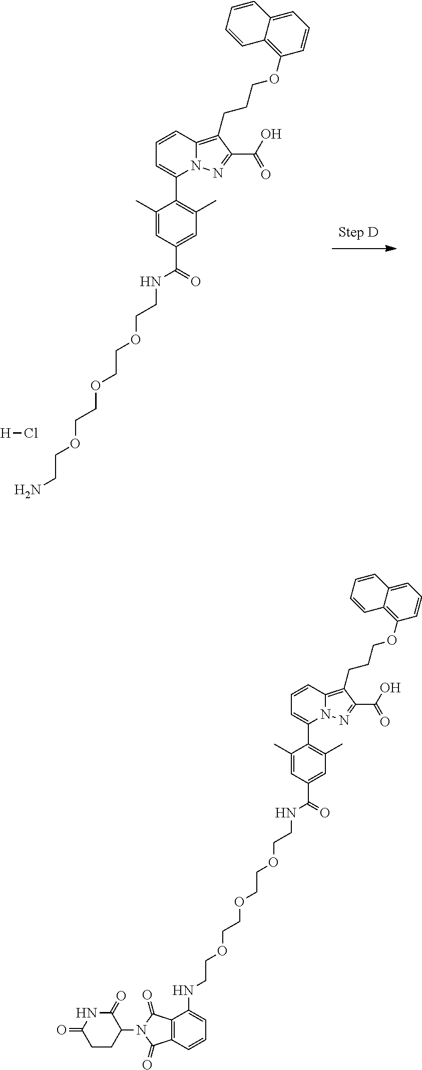 [SMILES: Cl ; Cc1cc(C(=O)NCCOCCOCCOCCN)cc(C)c1-c1cccc2c(CCCOc3cccc4ccccc34)c(C(=O)O)nn12 ; Cc1cc(C(=O)NCCOCCOCCOCCNc2cccc3c2C(=O)N(C2CCC(=O)NC2=O)C3=O)cc(C)c1-c1cccc2c(CCCOc3cccc4ccccc34)c(C(=O)O)nn12]

### Step A

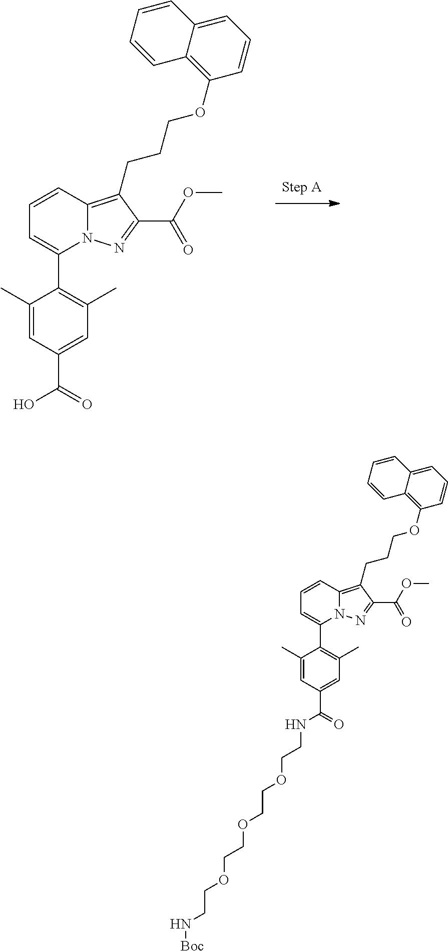 [SMILES: COC(=O)c1nn2c(-c3c(C)cc(C(=O)O)cc3C)cccc2c1CCCOc1cccc2ccccc12 ; COC(=O)c1nn2c(-c3c(C)cc(C(=O)NCCOCCOCCOCCNC(=O)OC(C)(C)C)cc3C)cccc2c1CCCOc1cccc2ccccc12]

4-(2-(methoxycarbonyl)-3-(3-(naphthalen-1-yloxy)propyl)pyrazolo[1,5-a]pyridin-7-yl)-3,5-dimethylbenzoic acid (100 mg, 0.20 mmol) was dissolved in anhydrous DMF (1.26 mL). DIPEA (103 L, 0.59 mmol) and HATU (90 mg, 0.24 mmol) were added under an inert atmosphere, and mixture was stirred in RT for 1 h. Then tert-butyl N-(2-{2-[2-(2-aminoethoxy)ethoxy]ethoxy}ethyl)carbamate (69 mg, 0.24 mmol) was added in solution of DMF (1.26 mL). The mixture was stirred at RT for overnight. Solvent was removed under reduced pressure, brine and DCM were added and product was extracted with DCM to give methyl 7-(4-((2,2-dimethyl-4-oxo-3,8,11,14-tetraoxa-5-azahexadecan-16-yl)carbamoyl)-2,6-dimethylphenyl)-3-(3-(naphthalen-1-yloxy)propyl)pyrazolo[1,5-a]pyridine-2-carboxylate (151 mg, crude) which was used without further purification to the next step.

LCMS (ESI+): m/z 683.3 [M+H−Boc]⁺

### Step B

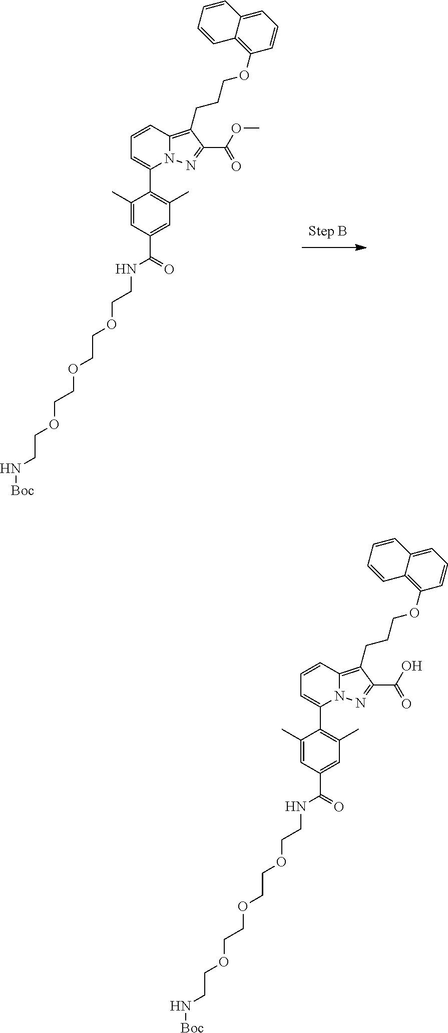 [SMILES: COC(=O)c1nn2c(-c3c(C)cc(C(=O)NCCOCCOCCOCCNC(=O)OC(C)(C)C)cc3C)cccc2c1CCCOc1cccc2ccccc12 ; Cc1cc(C(=O)NCCOCCOCCOCCNC(=O)OC(C)(C)C)cc(C)c1-c1cccc2c(CCCOc3cccc4ccccc34)c(C(=O)O)nn12]

To solution of methyl 7-(4-((2,2-dimethyl-4-oxo-3,8,11,14-tetraoxa-5-azahexadecan-16-yl)carbamoyl)-2,6-dimethylphenyl)-3-(3-(naphthalen-1-yloxy)propyl)pyrazolo[1,5-a]pyridine-2-carboxylate (151 mg, crude) in MeOH and THE (1:1, 2.12 mL) was added NaOH (77 mg, 1.93 mmol) dissolved in 0.5 mL of water. Mixture was stirred at 70° C. for 18. Organic solvents were removed under reduced pressure, and product was extracted from aqueous phase with DCM. 7-(4-((2,2-dimethyl-4-oxo-3,8,11,14-tetraoxa-5-azahexadecan-16-yl)carbamoyl)-2,6-dimethylphenyl)-3-(3-(naphthalen-1-yloxy)propyl)pyrazolo[1,5-a]pyridine-2-carboxylic acid (150 mg, crude) was used for next step without further purification.

LCMS (ESI−): m/z 787.7 [M−H]⁻

### Step C

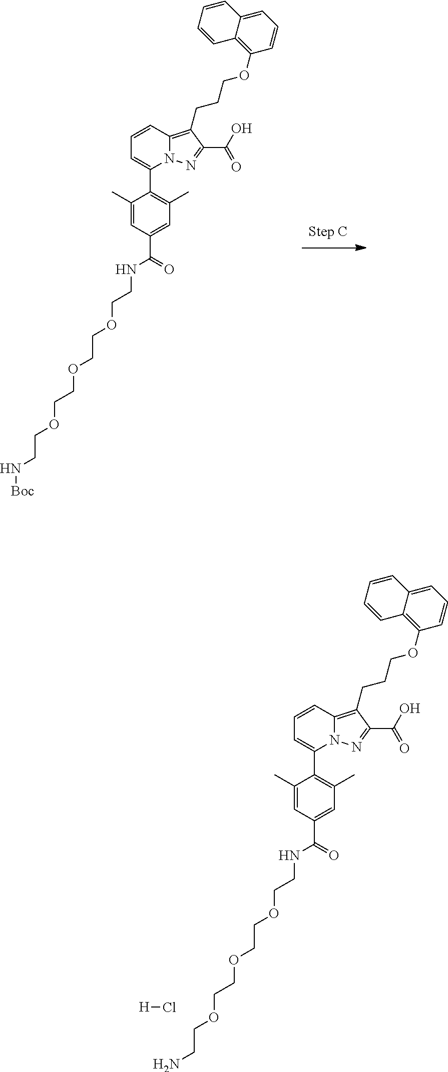 [SMILES: Cc1cc(C(=O)NCCOCCOCCOCCNC(=O)OC(C)(C)C)cc(C)c1-c1cccc2c(CCCOc3cccc4ccccc34)c(C(=O)O)nn12 ; Cl ; Cc1cc(C(=O)NCCOCCOCCOCCN)cc(C)c1-c1cccc2c(CCCOc3cccc4ccccc34)c(C(=O)O)nn12]

7-(4-((2,2-dimethyl-4-oxo-3,8,11,14-tetraoxa-5-azahexadecan-16-yl)carbamoyl)-2,6-dimethylphenyl)-3-(3-(naphthalen-1-yloxy)propyl)pyrazolo[1,5-a]pyridine-2-carboxylic acid (154 mg, crude) was dissolved in THE (0.98 mL) and 4M HCl in dioxane (197 μL, 0.79 mmol) was added. Mixture was stirred at RT overnight. Solvents were removed under reduced pressure, and crude co-evaporated three times with Et₂O. 7-{4-[(2-{2-[2-(2-aminoethoxy)ethoxy]ethoxy}ethyl)carbamoyl]-2,6-dimethylphenyl}-3-[3-(naphthalen-1-yloxy)propyl]pyrazolo[1,5-a]pyridine-2-carboxylic acid hydrochloride (140 mg, crude) was used for next step without further purification.

LCMS (ESI+): m/z 669.4 [M+H]⁺

### Step D

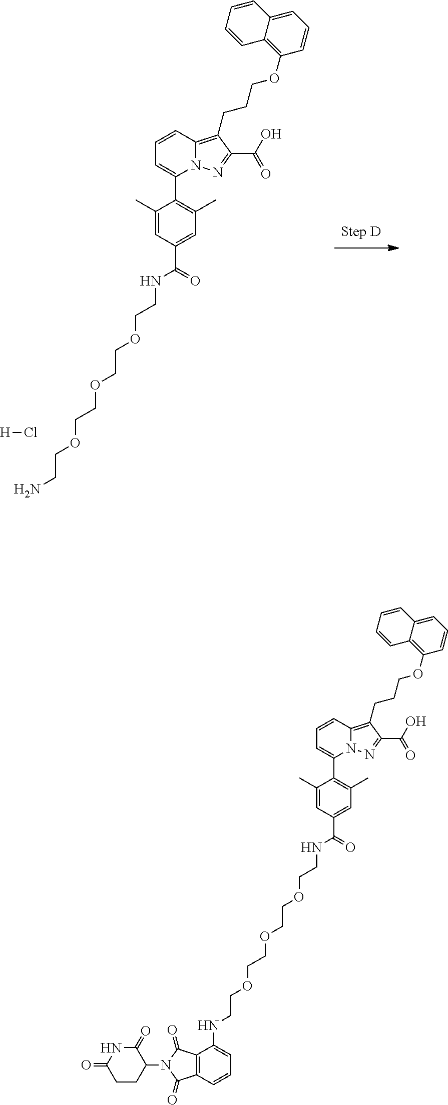 [SMILES: Cl ; Cc1cc(C(=O)NCCOCCOCCOCCN)cc(C)c1-c1cccc2c(CCCOc3cccc4ccccc34)c(C(=O)O)nn12 ; Cc1cc(C(=O)NCCOCCOCCOCCNc2cccc3c2C(=O)N(C2CCC(=O)NC2=O)C3=O)cc(C)c1-c1cccc2c(CCCOc3cccc4ccccc34)c(C(=O)O)nn12]

DIPEA (20 μL, 0.11 mmol) was added to a solution of 7-{4-[(2-{2-[2-(2-aminoethoxy)ethoxy]ethoxy}ethyl)carbamoyl]-2,6-dimethylphenyl}-3-[3-(naphthalen-1-yloxy)propyl]pyrazolo[1,5-a]pyridine-2-carboxylic acid hydrochloride (25 mg crude) and 2-(2,6-dioxopiperidin-3-yl)-4-fluoroisoindoline-1,3-dione (31 mg, 0.11 mmol) in DMSO (0.26 mL). The reaction mixture was heated at 90° C. for 20 h. Crude was purified by flash chromatography (SiO₂, 5% MeOH in DCM). 7-(4-((2-(2-(2-(2-((2-(2,6-dioxopiperidin-3-yl)-1,3-dioxoisoindolin-4-yl)amino)ethoxy)ethoxy)ethoxy)ethyl)carbamoyl)-2,6-dimethylphenyl)-3-(3-(naphthalen-1-yloxy)propyl)pyrazolo[1,5-a]pyridine-2-carboxylic acid (6.50 mg, 0.007 mmol, 4% over four steps) was isolated as yellow solid.

LCMS (ESI+): m/z 925.9 [M+H]⁺

¹H NMR (601 MHz, DMSO) δ 12.99 (s, 1H), 11.08 (s, 1H), 8.52 (t, J=5.6 Hz, 1H), 8.27-8.22 (m, 1H), 7.89-7.83 (m, 1H), 7.78 (d, J=8.5 Hz, 1H), 7.65 (s, 2H), 7.59-7.47 (m, 3H), 7.45 (d, J=8.3 Hz, 1H), 7.38 (t, J=7.9 Hz, 1H), 7.26-7.20 (m, 1H), 7.13 (d, J=8.6 Hz, 1H), 7.03 (d, J=7.0 Hz, 1H), 6.88 (d, J=7.6 Hz, 1H), 6.87-6.80 (m, 1H), 6.60 (t, J=5.8 Hz, 1H), 5.05 (dd, J=12.8, 5.5 Hz, 1H), 4.17 (t, J=6.1 Hz, 2H), 3.61 (t, J=5.6 Hz, 2H), 3.58-3.52 (m, 9H), 3.47-3.41 (m, 4H), 3.29-3.23 (m, 3H), 2.88 (ddd, J=17.0, 13.8, 5.4 Hz, 1H), 2.62-2.55 (m, 1H), 2.25-2.19 (m, 2H), 2.05-1.97 (m, 2H), 1.94 (s, 6H).

### Example 48. (2R)-2-[(5-{3-chloro-2-methyl-4-[2-(4-methylpiperazin-1-yl)ethoxy]phenyl}-6-(5-fluorofuran-2-yl)thieno[2,3-d]pyrimidin-4-yl)oxy]-N-(2-{3-[2-(2-{[2-(2,6-dioxopiperidin-3-yl)-1,3-dioxo-2,3-dihydro-1H-isoindol-4-yl]amino}ethoxy)ethoxy]propanamido}ethanesulfonyl)-3-(2-{[1-(2,2,2-trifluoroethyl)-1H-pyrazol-5-yl]methoxy}phenyl)propanamide (260)

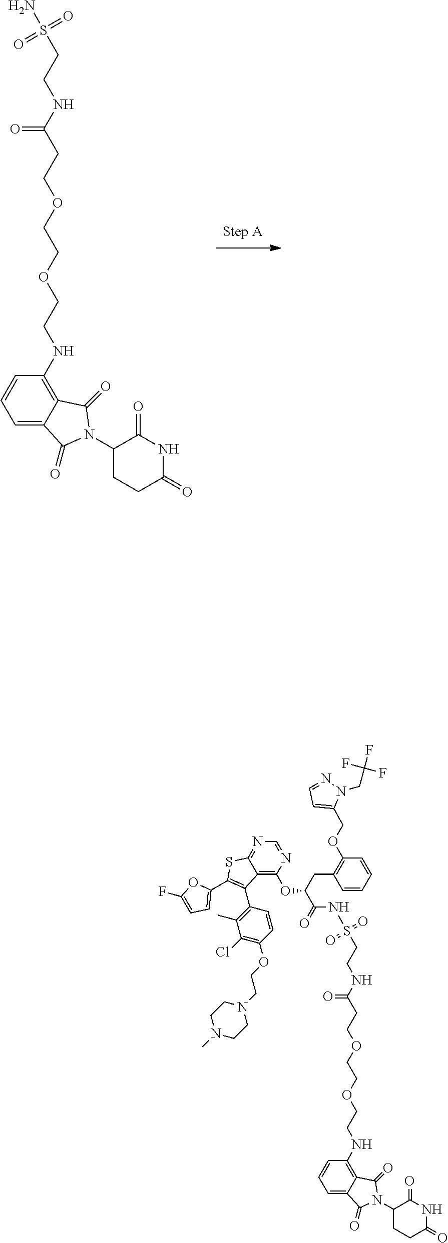 [SMILES: NS(=O)(=O)CCNC(=O)CCOCCOCCNc1cccc2c1C(=O)N(C1CCC(=O)NC1=O)C2=O ; Cc1c(-c2c(-c3ccc(F)o3)sc3ncnc(O[C@H](Cc4ccccc4OCc4ccnn4CC(F)(F)F)C(=O)NS(=O)(=O)CCNC(=O)CCOCCOCCNc4cccc5c4C(=O)N(C4CCC(=O)NC4=O)C5=O)c23)ccc(OCCN2CCN(C)CC2)c1Cl]

### Step A

To a stirred solution of (R)-2-((5-(3-chloro-2-methyl-4-(2-(4-methylpiperazin-1-yl)ethoxy)phenyl)-6-(5-fluorofuran-2-yl)thieno[2,3-d]pyrimidin-4-yl)oxy)-3-(2-((1-(2,2,2-trifluoroethyl)-1H-pyrazol-5-yl)methoxy)phenyl)propanoic acid (15.0 mg, 0.018 mmol) in DCM (0.181 mL) were added EDC (4.2 mg, 0.022 mmol), DMAP (4.4 mg, 0.036 mmol), triethylamine (0.013 mL, 0.090 mmol), and 3-[2-(2-{[2-(2,6-dioxopiperidin-3-yl)-1,3-dioxo-2,3-dihydro-1H-isoindol-4-yl]amino}ethoxy)ethoxy]-N-(2-sulfamoylethyl)propanamide (12.7 mg, 0.024 mmol). The reaction mixture was stirred for 15 h at ambient temperature then for overnight in 60° C. Solvents were removed under reduced pressure. Desired product was purified using flash chromatography (SiO₂, DCM:MeOH, 0-10%), then obtained residue was purified using preparative TLC (SiO₂ 10% MeOH in DCM). Final purification by reverse phase preparative HPLC (H₂O:MeCN+0.1% FA) resulted (2R)-2-[(5-{3-chloro-2-methyl-4-[2-(4-methylpiperazin-1-yl)ethoxy]phenyl}-6-(5-fluorofuran-2-yl)thieno[2,3-d]pyrimidin-4-yl)oxy]-N-(2-{3-[2-(2-{[2-(2,6-dioxopiperidin-3-yl)-1,3-dioxo-2,3-dihydro-1H-isoindol-4-yl]amino}ethoxy)ethoxy]propanamido}ethanesulfonyl)-3-(2-{[1-(2,2,2-trifluoroethyl)-1H-pyrazol-5-yl]methoxy}phenyl)propanamide (1.0 mg, 0.001 mmol, 4.1%) as a yellow solid.

LCMS (ESI+): m/z 1349.6 [M+H]⁺

¹H NMR (500 MHz, DMSO) δ 11.08 (s, 1H), 8.45 (s, 1H), 7.82-7.64 (m, 1H), 7.56-7.52 (m, 2H), 7.25-7.16 (m, 1H), 7.16-7.07 (m, 4H), 7.07-6.97 (m, 3H), 6.82 (s, 1H), 6.70 (t, J=7.4 Hz, 1H), 6.60 (t, J=5.8 Hz, 1H), 5.84 (dd, J=6.8, 3.7 Hz, 2H), 5.67 (t, J=3.5 Hz, 1H), 5.35-5.14 (m, 7H), 5.06 (dd, J=12.7, 5.4 Hz, 1H), 4.37-4.26 (m, 1H), 4.26-4.18 (m, 1H), 3.60 (q, J=6.6, 6.0 Hz, 6H), 3.57-3.53 (m, 3H), 3.53-3.48 (m, 3H), 3.46 (q, J=5.7 Hz, 3H), 3.13-3.08 (m, 1H), 2.96-2.81 (m, 6H), 2.62-2.60 (m, 2H), 2.40-2.35 (m, 4H), 2.27 (t, J=6.5 Hz, 2H), 2.15-1.98 (m, 2H).

### Example 49. N-{2-[({17-chloro-5,13,14,22-tetramethyl-28-oxa-2,9-dithia-5,6,12,13,22-pentaazaheptacyclo[27.7.1.1⁴,⁷.0¹¹,¹⁵.0,¹⁶,²¹.0²⁰,²⁴.0³⁰,³⁵]octatriaconta-1(37),4(38),6,11,14,16,18,20,23,29,31,33,35-tridecaen-23-yl}formamido)sulfonyl]ethyl}-3-[2-(2-{[2-(2,6-dioxopiperidin-3-yl)-1,3-dioxo-2,3-dihydro-1H-isoindol-4-yl]amino}ethoxy)ethoxy]propanamide (261)

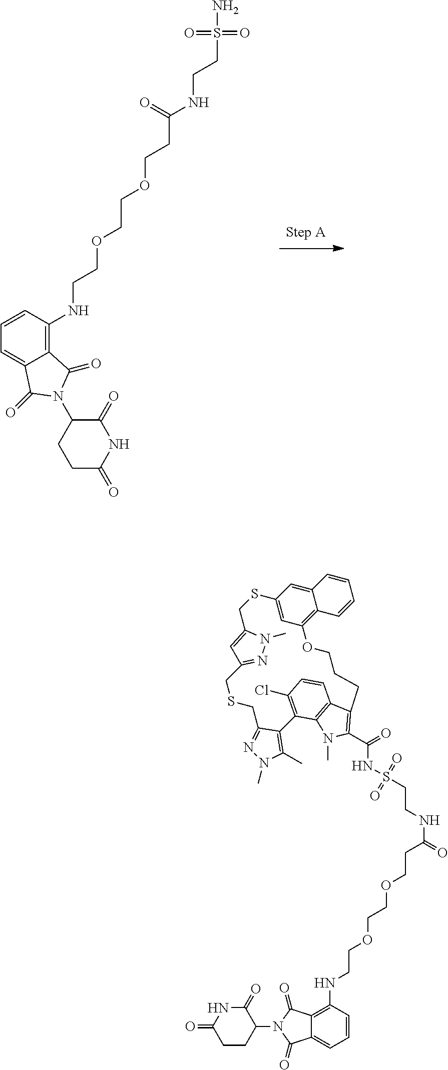 [SMILES: NS(=O)(=O)CCNC(=O)CCOCCOCCNc1cccc2c1C(=O)N(C1CCC(=O)NC1=O)C2=O ; Cc1c2c(nn1C)CSCc1cc(n(C)n1)CSc1cc(c3ccccc3c1)OCCCc1c(C(=O)NS(=O)(=O)CCNC(=O)CCOCCOCCNc3cccc4c3C(=O)N(C3CCC(=O)NC3=O)C4=O)n(C)c3c-2c(Cl)ccc13]

### Step A

To a stirred solution of 17-chloro-5,13,14,22-tetramethyl-28-oxa-2,9-dithia-5,6,12,13,22-pentaazaheptacyclo[27.7.1.1⁴,⁷.0¹¹,¹⁵.0¹⁶,²¹.0²⁰,²⁴.0³⁰,³⁵]octatriaconta-1(37),4(38),6,11,14,16,18,20,23,29,31,33,35-tridecaene-23-carboxylic acid (15.0 mg, 0.022 mmol) in DCM (0.223 mL) were added EDC (4.3 mg, 0.022 mmol), DMAP (5.5 mg, 0.045 mmol), triethylamine (0.016 mL, 0.112 mmol) and 3-[2-(2-{[2-(2,6-dioxopiperidin-3-yl)-1,3-dioxo-2,3-dihydro-1H-isoindol-4-yl]amino}ethoxy)ethoxy]-N-(2-sulfamoylethyl)propanamide (15.7 mg, 0.029 mmol) The reaction mixture was stirred for 15 h at ambient temperature then for overnight in 60° C. The crude was concentrated in vacuo and the residue was purified by reverse phase preparative HPLC (H₂O:CH₃CN+0.1% FA) to give a corresponding N-{2-[({17-chloro-5,13,14,22-tetramethyl-28-oxa-2,9-dithia-5,6,12,13,22-pentaazaheptacyclo[27.7.1.1⁴,⁷.0¹¹,¹⁵.0¹⁶,²¹.0²⁰,²⁴.0³⁰,35]octatriaconta-1(37),4(38),6,11,14,16,18,20,23,29,31,33,35-tridecaen-23-yl}formamido)sulfonyl]ethyl}-3-[2-(2-{[2-(2,6-dioxopiperidin-3-yl)-1,3-dioxo-2,3-dihydro-1H-isoindol-4-yl]amino}ethoxy)ethoxy]propanamide (4.7 mg, 0.004 mmol, 17.3%) as yellow solid.

LCMS (ESI+): m/z 1193.1 [M+H]⁺

¹H NMR (500 MHz, DMSO) δ 12.40 (s, 1H), 11.07 (s, 1H), 8.19-7.97 (m, 2H), 7.81 (s, 1H), 7.68 (d, J=8.1 Hz, 1H), 7.56 (dd, J=8.6, 7.1 Hz, 1H), 7.44 (dt, J=19.5, 7.2 Hz, 2H), 7.33 (s, 1H), 7.25-7.09 (m, 2H), 7.02 (d, J=7.0 Hz, 1H), 6.73 (s, 1H), 6.59 (t, J=5.8 Hz, 1H), 5.13-4.87 (m, 2H), 4.39 (s, 1H), 4.20 (d, J=15.4 Hz, 1H), 3.97 (s, 2H), 3.76 (s, 3H), 3.71 (s, 3H), 3.62-3.56 (m, 5H), 3.55-3.37 (m, 12H), 3.15 (d, J=13.0 Hz, 2H), 3.10 (s, 1H), 2.97 (d, J=13.7 Hz, 1H), 2.87 (ddd, J=16.7, 13.7, 5.4 Hz, 1H), 2.62-2.52 (m, 2H), 2.30 (t, J=6.4 Hz, 4H), 2.06-1.96 (m, 5H).

### Example 50. 2-((4-(3-(4-(3-((2-(2,6-dioxopiperidin-3-yl)-1,3-dioxoisoindolin-4-yl)amino)propyl)piperazin-1-yl)propoxy)phenyl)sulfonamido)-4-(4-methylnaphthalen-1-yl)benzoic acid (257)

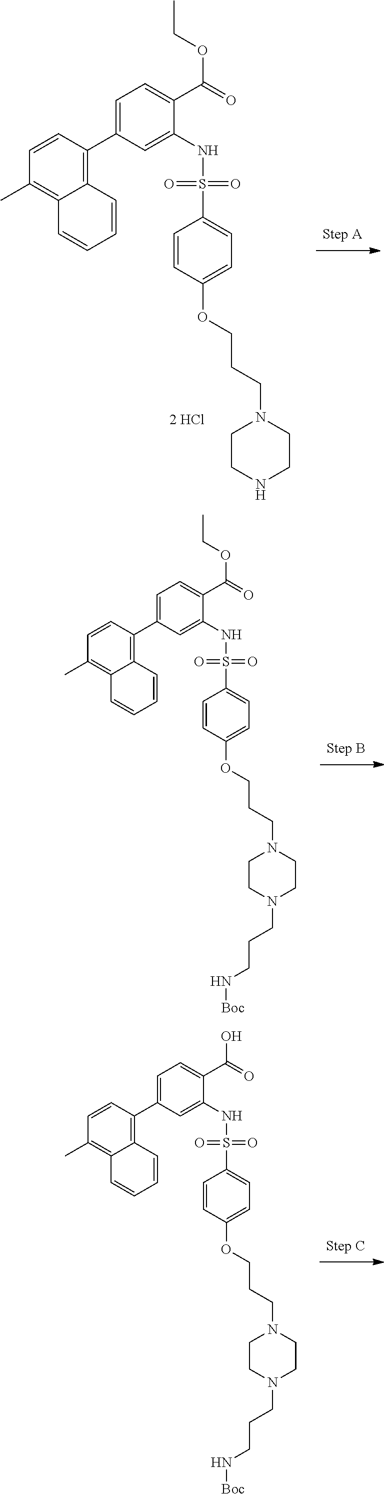

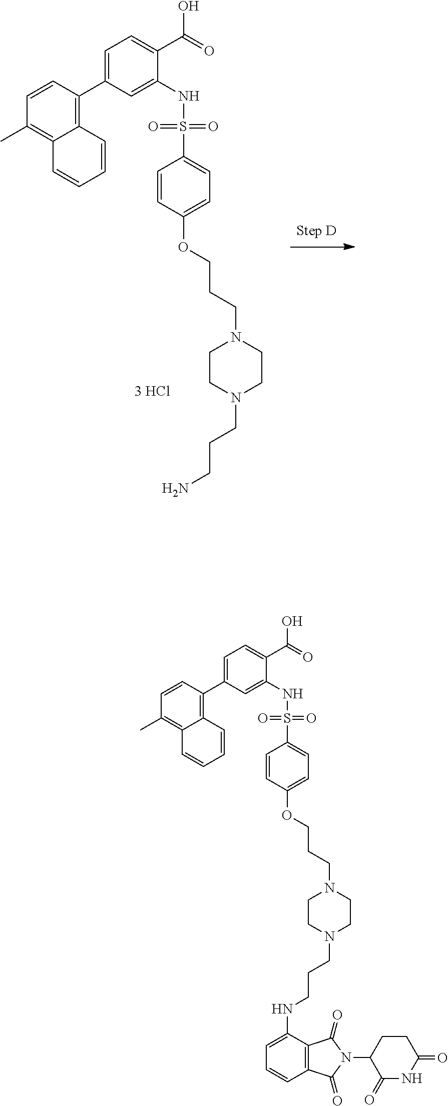 [SMILES: Cc1ccc(-c2ccc(C(=O)O)c(NS(=O)(=O)c3ccc(OCCCN4CCN(CCCN)CC4)cc3)c2)c2ccccc12 ; Cc1ccc(-c2ccc(C(=O)O)c(NS(=O)(=O)c3ccc(OCCCN4CCN(CCCNc5cccc6c5C(=O)N(C5CCC(=O)NC5=O)C6=O)CC4)cc3)c2)c2ccccc12]

### Step A

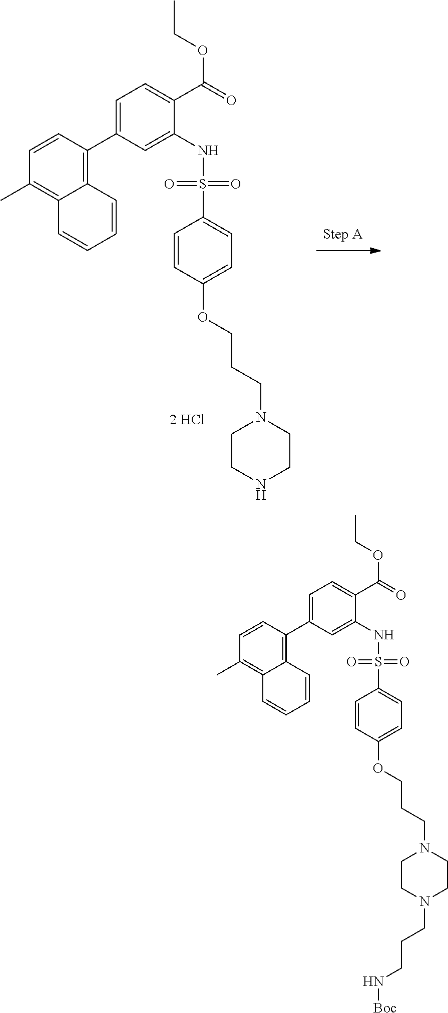 [SMILES: CCOC(=O)c1ccc(-c2ccc(C)c3ccccc23)cc1NS(=O)(=O)c1ccc(OCCCN2CCNCC2)cc1 ; CCOC(=O)c1ccc(-c2ccc(C)c3ccccc23)cc1NS(=O)(=O)c1ccc(OCCCN2CCN(CCCNC(=O)OC(C)(C)C)CC2)cc1]

Ethyl 4-(4-methylnaphthalen-1-yl)-2-((4-(3-(piperazin-1-yl)propoxy)phenyl)sulfonamido)benzoate (dihydrochloride salt) (100 mg, 0.151 mmol) and tert-butyl (3-oxopropyl)carbamate (59 mg, 0.340 mmol) were dissolved in DCM (1.5 mL). NaBH(OAc)₃ (72 mg 0.340 mmol) was added in one portion and the mixture was stirred for 16 h, whereupon LCMS and TLC indicated the reaction was complete. The reaction mixture was diluted with DCM and washed with saturated NaHCO₃. The aqueous phase was extracted with DCM, the organic phases were combined and dried over Na₂SO₄. Solvent was evaporated and crude product purified with flash column chromatography (SiO₂, MeOH:DCM, 0-10%) to afford ethyl 2-((4-(3-(4-(3-((tert-butoxycarbonyl)amino)propyl)piperazin-1-yl)propoxy)phenyl)sulfonamido)-4-(4-methylnaphthalen-1-yl)benzoate as an opaque oil (103 mg, 91%).

### Step B

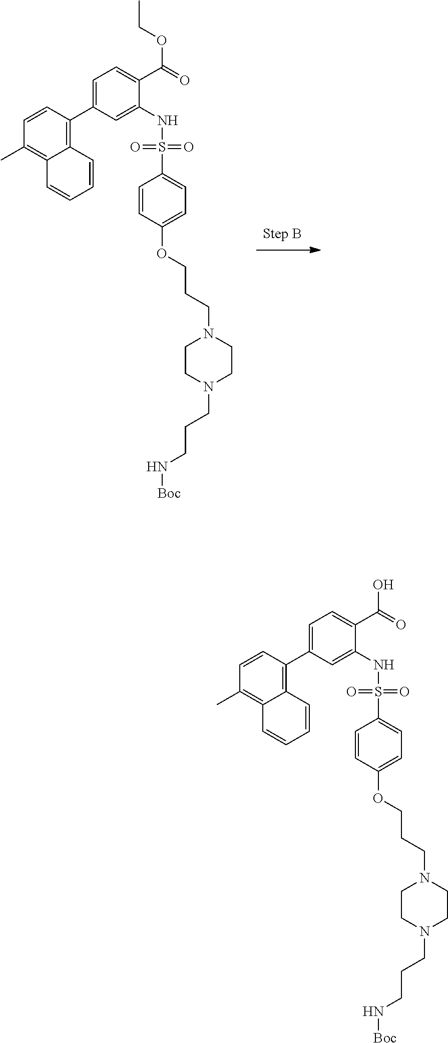 [SMILES: CCOC(=O)c1ccc(-c2ccc(C)c3ccccc23)cc1NS(=O)(=O)c1ccc(OCCCN2CCN(CCCNC(=O)OC(C)(C)C)CC2)cc1 ; Cc1ccc(-c2ccc(C(=O)O)c(NS(=O)(=O)c3ccc(OCCCN4CCN(CCCNC(=O)OC(C)(C)C)CC4)cc3)c2)c2ccccc12]

Ethyl 2-((4-(3-(4-(3-((tert-butoxycarbonyl)amino)propyl)piperazin-1-yl)propoxy)phenyl)sulfonamido)-4-(4-methylnaphthalen-1-yl)benzoate (90 mg, 0.121 mmol) was dissolved in THE/water (1 mL, 1:1). NaOH (24 mg, 0.604 mmol) was added in one portion and the mixture stirred for 18 h. The reaction mixture was concentrated in vacuo. The resulting solid was dissolved in a small amount of H₂O and the mixture neutralized with 1M HCl solution. The aqueous phase was extracted several times with 10% MeOH in CH₂Cl₂, the organics combined and dried over Na₂SO₄. Crude product was purified with flash column chromatography (SiO₂, MeOH:DCM, 5-50%) to afford 2-((4-(3-(4-(3-((tert-butoxycarbonyl)amino)propyl)piperazin-1-yl)propoxy)phenyl)sulfonamido)-4-(4-methylnaphthalen-1-yl)benzoic acid as an off-white solid (42 mg, 0.058, 49%).

### Step C

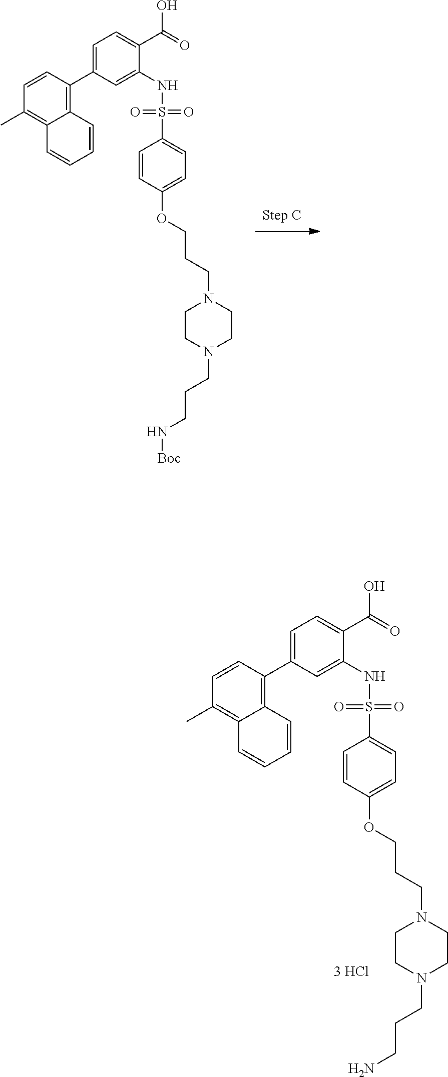 [SMILES: Cc1ccc(-c2ccc(C(=O)O)c(NS(=O)(=O)c3ccc(OCCCN4CCN(CCCNC(=O)OC(C)(C)C)CC4)cc3)c2)c2ccccc12 ; Cc1ccc(-c2ccc(C(=O)O)c(NS(=O)(=O)c3ccc(OCCCN4CCN(CCCN)CC4)cc3)c2)c2ccccc12]

2-((4-(3-(4-(3-((tert-butoxycarbonyl)amino)propyl)piperazin-1-yl)propoxy)phenyl)sulfonamido)-4-(4-methylnaphthalen-1-yl)benzoic acid (38 mg, 0.053 mmol) was dissolved in 4M HCl in dioxane (1 mL) at 0° C. and the mixture was stirred for 2 h, allowing for slow warming to room temperature, whereupon LCMS indicated the reaction was complete. The reaction mixture was concentrated in vacuo, and triturated with Et₂O to afford 2-((4-(3-(4-(3-aminopropyl)piperazin-1-yl)propoxy)phenyl)sulfonamido)-4-(4-methylnaphthalen-1-yl)benzoic acid trihydrochloride as a beige solid (38 mg, 0.052 mmol, 99%).

### Step D

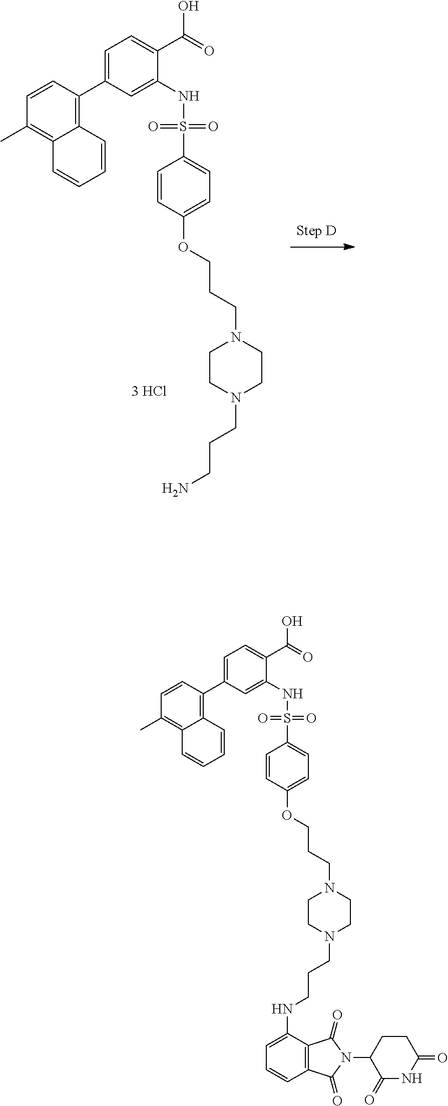 [SMILES: Cc1ccc(-c2ccc(C(=O)O)c(NS(=O)(=O)c3ccc(OCCCN4CCN(CCCN)CC4)cc3)c2)c2ccccc12 ; Cc1ccc(-c2ccc(C(=O)O)c(NS(=O)(=O)c3ccc(OCCCN4CCN(CCCNc5cccc6c5C(=O)N(C5CCC(=O)NC5=O)C6=O)CC4)cc3)c2)c2ccccc12]

2-((4-(3-(4-(3-aminopropyl)piperazin-1-yl)propoxy)phenyl)sulfonamido)-4-(4-methylnaphthalen-1-yl)benzoic acid trihydrochloride (38 mg, 0.052 mmol) and 2-(2,6-dioxopiperidin-3-yl)-4-fluoroisoindoline-1,3-dione (14.5 mg, 0.052 mmol) were dissolved in DMSO (0.2 mL). DIPEA (36.5 L, 0.209 mmol) was added and the mixture was stirred for until full conversion (monitored with LCMS) at 90° C. under argon. The reaction mixture was concentrated in vacuo and the crude product purified with flash column chromatography to afford 2-((4-(3-(4-(3-((2-(2,6-dioxopiperidin-3-yl)-1,3-dioxoisoindolin-4-yl)amino)propyl)piperazin-1-yl)propoxy)phenyl)sulfonamido)-4-(4-methylnaphthalen-1-yl)benzoic acid as a yellow solid (1.98 mg, 0.002 mmol. 4.3% yield).

LCMS (ESI+): m/z 873.9 [M+H]⁺

¹H NMR (500 MHz, DMSO) δ 11.10 (s, 1H), 8.10 (dd, J=8.8, 1.3 Hz, 1H), 7.96 (d, J=7.9 Hz, 1H), 7.68 (d, J=9.1 Hz, 2H), 7.64-7.54 (m, 3H), 7.52-7.39 (m, 3H), 7.24 (d, J=7.1 Hz, 1H), 7.12 (d, J=8.6 Hz, 1H), 7.09-7.01 (m, 3H), 6.94 (d, J=7.7 Hz, 1H), 6.89-6.80 (m, 1H), 5.05 (dd, J=12.9, 6.2 Hz, 1H), 4.10 (t, J=6.2 Hz, 2H), 3.41-3.33 (m, 4H), 3.30-3.27 (m, 6H), 2.88 (ddd, J=16.9, 13.8, 5.4 Hz, 1H), 2.70 (s, 3H), 2.63-2.58 (m, 1H), 2.58-2.54 (m, 1H), 2.54-2.51 (m, 2H), 2.49-2.41 (m, 2H), 2.11-1.92 (m, 3H), 1.83 (bs, 2H).

### Example 51. 2-((4-(3-(4-((2-(2,6-dioxopiperidin-3-yl)-1,3-dioxoisoindolin-4-ylglycyl)piperazin-1-yl)propoxy)phenyl)sulfonamido)-4-(4-methylnaphthalen-1-yl)benzoic acid (258)

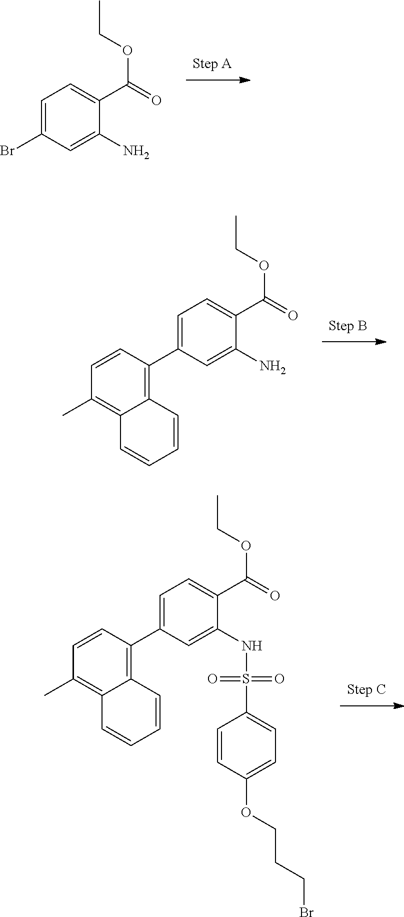 [SMILES: CCOC(=O)c1ccc(Br)cc1N ; CCOC(=O)c1ccc(-c2ccc(C)c3ccccc23)cc1N ; CCOC(=O)c1ccc(-c2ccc(C)c3ccccc23)cc1NS(=O)(=O)c1ccc(OCCCBr)cc1]

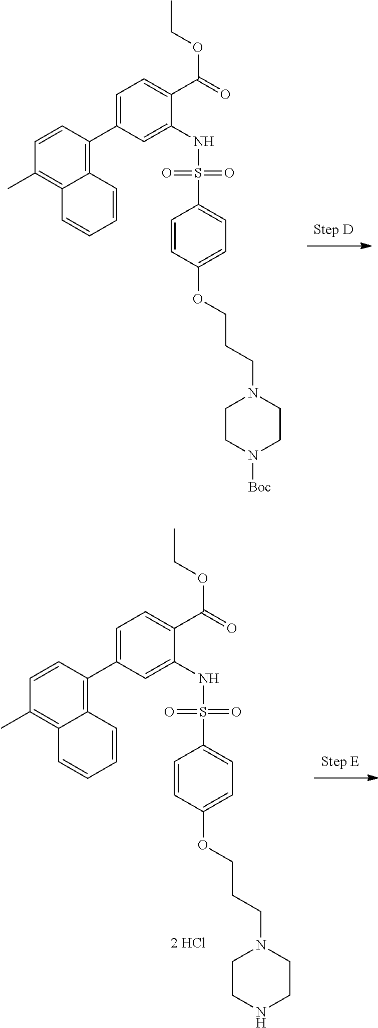 [SMILES: CCOC(=O)c1ccc(-c2ccc(C)c3ccccc23)cc1NS(=O)(=O)c1ccc(OCCCN2CCN(C(=O)OC(C)(C)C)CC2)cc1 ; CCOC(=O)c1ccc(-c2ccc(C)c3ccccc23)cc1NS(=O)(=O)c1ccc(OCCCN2CCNCC2)cc1]

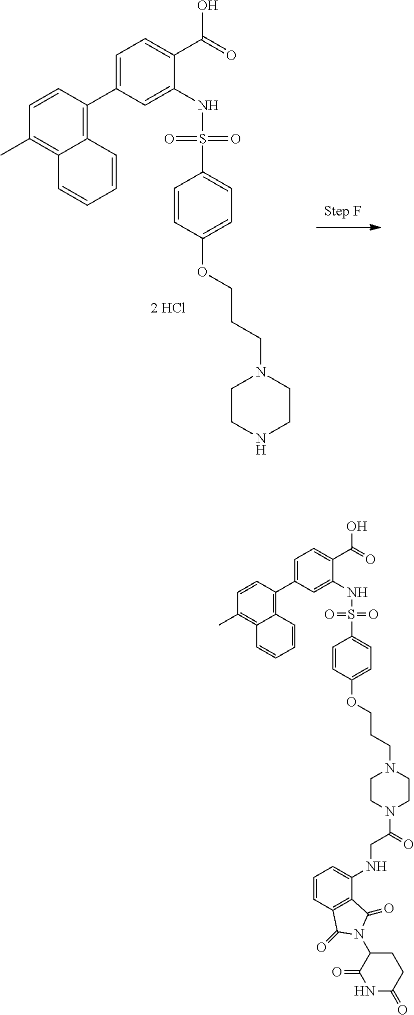 [SMILES: Cc1ccc(-c2ccc(C(=O)O)c(NS(=O)(=O)c3ccc(OCCCN4CCNCC4)cc3)c2)c2ccccc12 ; Cc1ccc(-c2ccc(C(=O)O)c(NS(=O)(=O)c3ccc(OCCCN4CCN(C(=O)CNc5cccc6c5C(=O)N(C5CCC(=O)NC5=O)C6=O)CC4)cc3)c2)c2ccccc12]

### Step A

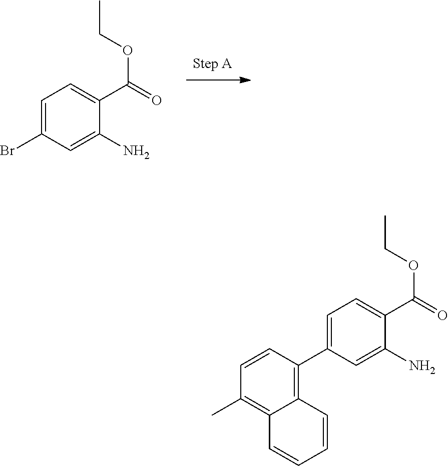 [SMILES: CCOC(=O)c1ccc(Br)cc1N ; CCOC(=O)c1ccc(-c2ccc(C)c3ccccc23)cc1N]

To a solution of ethyl 2-amino-4-bromobenzoate (14.1 g, 57.8 mmol) in mixture of dioxane and H₂O (4:1) was added (4-methylnaphthalen-1-yl)boronic acid (16.2 g, 87.1 mmol), Na₂CO₃ (18.4 g, 174 mmol), and Pd(PPh₃)₄ (2 g, 1.73 mmol) under argon atmosphere. The mixture was heated at 90° C. for 24 h. Then it was cooled down to room temperature, poured in H₂O (500 mL), and extracted with EtOAc (3×200 mL). The combined organic phases were washed with H₂O and brine, and dried over anhydrous sodium sulfate. The organic phase was concentrated and purified by column chromatography to give ethyl 2-amino-4-(4-methylnaphthalen-1-yl)benzoate (13.7 g, 44.9 mmol, 78% yield).

### Step B

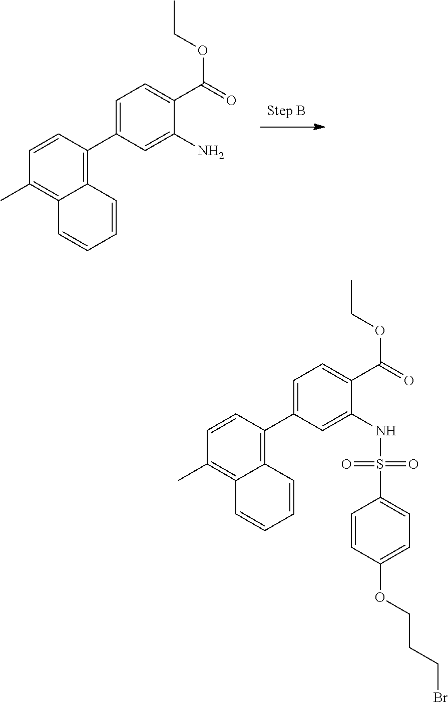 [SMILES: CCOC(=O)c1ccc(-c2ccc(C)c3ccccc23)cc1N ; CCOC(=O)c1ccc(-c2ccc(C)c3ccccc23)cc1NS(=O)(=O)c1ccc(OCCCBr)cc1]

A solution of 4-(3-bromopropoxy)benzenesulfonyl chloride (16.7 g, 53.3 mmol) in anhydrous THE (50 mL) was slowly added to a solution of ethyl 2-amino-4-(4-methylnaphthalen-1-yl)benzoate (11.2 g, 36.7 mmol) in the mixture of pyridine and THE (1:2, 100 mL) maintaining temperature below −5° C. The mixture was slowly warmed to room temperature and stirred overnight. Then, it was poured in H₂O and extracted with EtOAc (3×). The combined organic layers were washed with 10% HCl, H₂O, aq. solution of NaHCO₃, brine and dried over Na₂SO₄. The solution was evaporated under reduced pressure to give ethyl 2-((4-(3-bromopropoxy)phenyl)sulfonamido)-4-(4-methylnaphthalen-1-yl)benzoate (11.2 g, 19.2 mmol, 57.6% yield).

### Step C

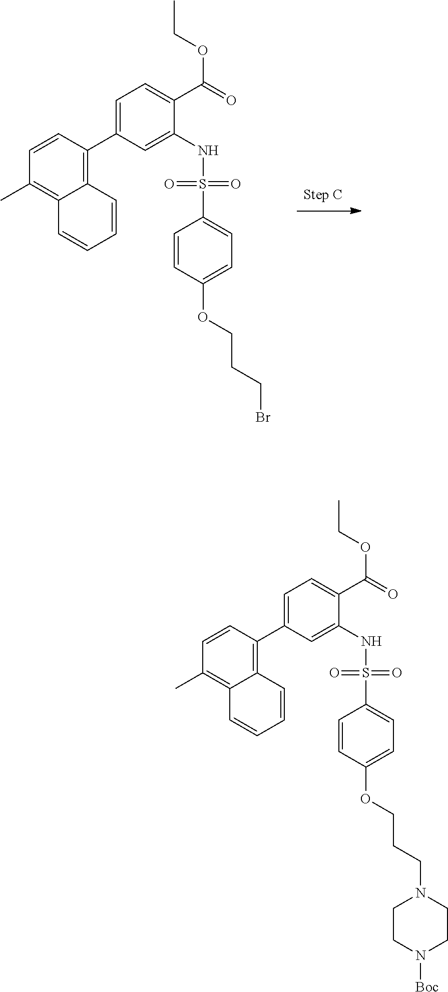 [SMILES: CCOC(=O)c1ccc(-c2ccc(C)c3ccccc23)cc1NS(=O)(=O)c1ccc(OCCCBr)cc1 ; CCOC(=O)c1ccc(-c2ccc(C)c3ccccc23)cc1NS(=O)(=O)c1ccc(OCCCN2CCN(C(=O)OC(C)(C)C)CC2)cc1]

A mixture of ethyl 2-((4-(3-bromopropoxy)phenyl)sulfonamido)-4-(4-methylnaphthalen-1-yl)benzoate (11.2 g, 19.2 mmol), tert-butyl piperazine-1-carboxylate (10.7 g, 57.4 mmol), and K₂CO₃ (2.8 g, 20.3 mmol) in N-methyl pyrrolidone (100 mL) was heated at 100° C. overnight. Then, it was cooled down to room temperature, poured into H₂O (200 mL), and extracted with EtOAc (3×). The combined organic layers were washed with H₂O and brine, dried over Na₂SO₄, and evaporated under reduced pressure to give tert-butyl 4-(3-(4-(N-(2-(ethoxycarbonyl)-5-(4-methylnaphthalen-1-yl)phenyl)sulfamoyl)phenoxy)propyl)piperazine-1-carboxylate (9.8 g, 14.2 mmol, 74% yield).

### Step D

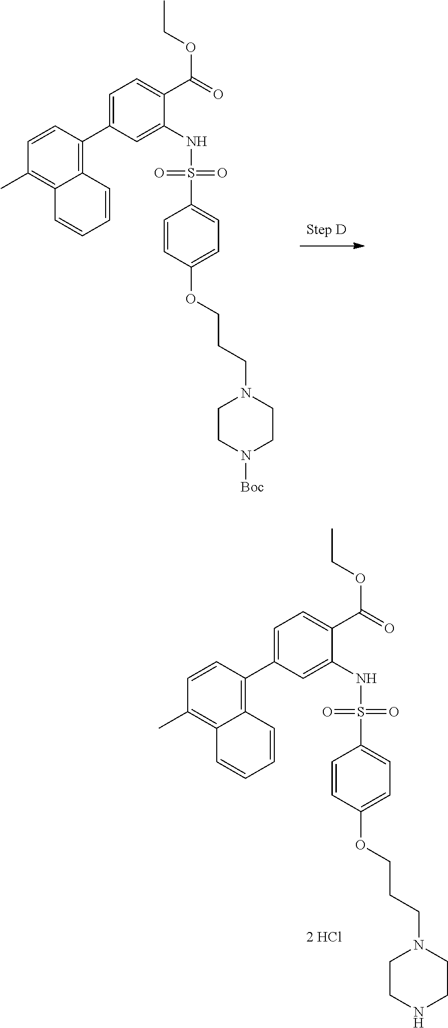 [SMILES: CCOC(=O)c1ccc(-c2ccc(C)c3ccccc23)cc1NS(=O)(=O)c1ccc(OCCCN2CCN(C(=O)OC(C)(C)C)CC2)cc1 ; CCOC(=O)c1ccc(-c2ccc(C)c3ccccc23)cc1NS(=O)(=O)c1ccc(OCCCN2CCNCC2)cc1]

Dry HCl (gas) was slowly bubbled through a solution of tert-butyl 4-(3-(4-(N-(2-(ethoxycarbonyl)-5-(4-methylnaphthalen-1-yl)phenyl)sulfamoyl)phenoxy)propyl)piperazine-1-carboxylate (9.8 g, 14.2 mmol) in dry CHCl₃ at 0° C. The mixture was then slowly warmed to room temperature and left stirring overnight. The precipitate was filtered, washed with CHCl₃ and hexane, and dried under reduced pressure to give ethyl 4-(4-methylnaphthalen-1-yl)-2-((4-(3-(piperazin-1-yl)propoxy)phenyl)sulfonamido)benzoate (5.5 g, 8.24 mmol, 58% yields) as dihydrochloride salt.

LCMS (ESI+): m/z 588.4 [M+H]⁺

### Step E

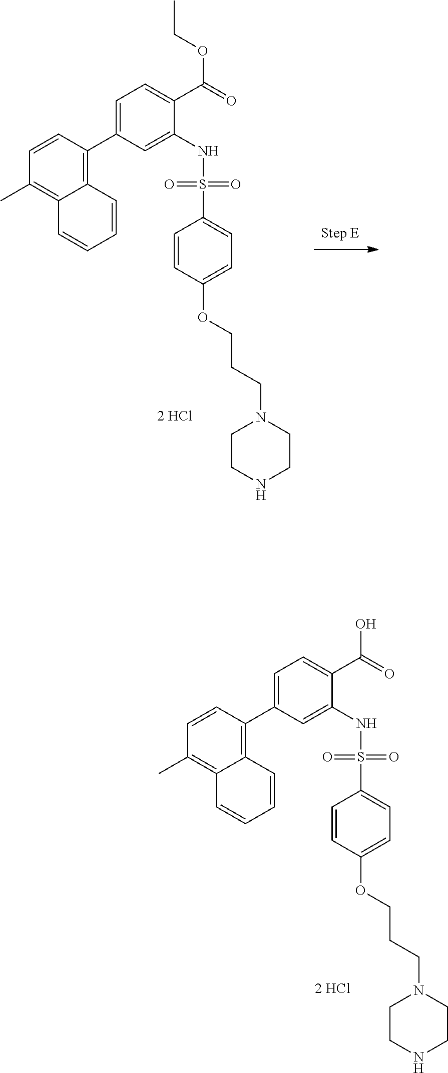 [SMILES: CCOC(=O)c1ccc(-c2ccc(C)c3ccccc23)cc1NS(=O)(=O)c1ccc(OCCCN2CCNCC2)cc1 ; Cc1ccc(-c2ccc(C(=O)O)c(NS(=O)(=O)c3ccc(OCCCN4CCNCC4)cc3)c2)c2ccccc12]

Ethyl 4-(4-methylnaphthalen-1-yl)-2-((4-(3-(piperazin-1-yl)propoxy)phenyl)sulfonamido)benzoate (dichloride salt) (25.0 mg, 0.0378 mmol) and LiOH (5.1 mg, 0.213 mmol) were dissolved in THE (0.500 mL) and water (0.500 mL) and the mixture was stirred at 60° C. for 16 h, whereupon LCMS indicated the reaction was complete. THE was removed under reduced pressure and the aqueous solution washed with DCM (2×). The aqueous phase was neutralized by the addition of aq. 1M HCl and concentrated under reduced pressure to afford 4-(4-methylnaphthalen-1-yl)-2-((4-(3-(piperazin-1-yl)propoxy)phenyl)sulfonamido)benzoic acid (23.8 mg, crude) as dihydrochloride salt as a white solid which was used without further purification.

LCMS (ESI+): m/z 560.9 [M+H]⁺

### Step F

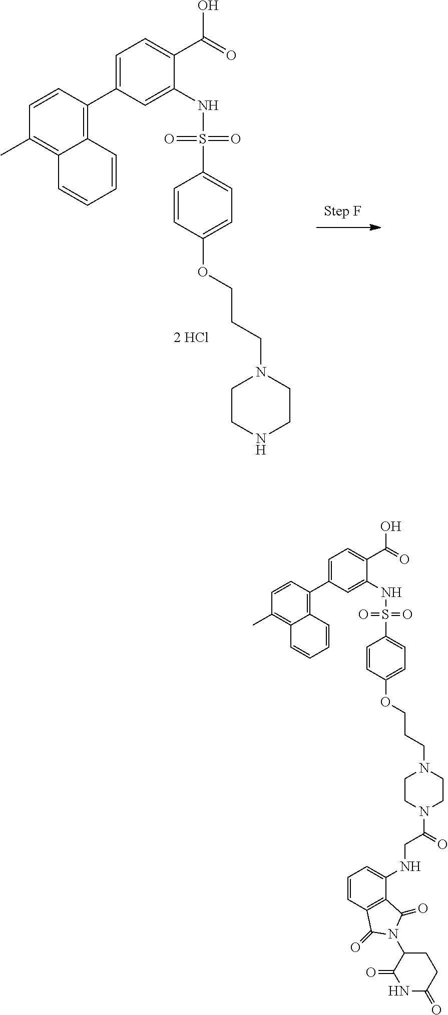 [SMILES: Cc1ccc(-c2ccc(C(=O)O)c(NS(=O)(=O)c3ccc(OCCCN4CCNCC4)cc3)c2)c2ccccc12 ; Cc1ccc(-c2ccc(C(=O)O)c(NS(=O)(=O)c3ccc(OCCCN4CCN(C(=O)CNc5cccc6c5C(=O)N(C5CCC(=O)NC5=O)C6=O)CC4)cc3)c2)c2ccccc12]

(2-(2,6-Dioxopiperidin-3-yl)-1,3-dioxoisoindolin-4-yl)glycine hydrochloride (17.2 mg, 0.047 mmol) and HATU (19.4 mg, 0.510 mmol) were dissolved in DMF (0.500 mL) and the mixture stirred for 16 h. LCMS (morpholine quench) indicated incomplete consumption of the starting material. Another portion of HATU (19.4 mg, 0.510 mmol) was added and the mixture stirred for 1 h, whereupon LCMS (morpholine quench) indicated the formation of the active ester was complete. 4-(4-Methylnaphthalen-1-yl)-2-((4-(3-(piperazin-1-yl)propoxy)phenyl)sulfonamido)benzoic acid dihydrochloride salt (23.8 mg of crude) was added and the reaction mixture was stirred for 16 h. The reaction mixture was concentrated in vacuo and purified by flash chromatography (SiO₂, MeOH:DCM, 4-20%) to afford 2-((4-(3-(4-((2-(2,6-dioxopiperidin-3-yl)-1,3-dioxoisoindolin-4-yl)glycyl)piperazin-1-yl)propoxy)phenyl)sulfonamido)-4-(4-methylnaphthalen-1-yl)benzoic acid (24.0 mg, 0.027 mmol, 73% over 2 steps) as a yellow solid.

LCMS (ESI+): m/z 874.4 [M+H]⁺

¹H NMR (500 MHz, DMSO) δ 12.88 (s, 1H), 11.09 (s, 1H), 8.12 (d, J=8.4 Hz, 1H), 8.02 (d, J=8.0 Hz, 1H), 7.73 (d, J=8.8 Hz, 2H), 7.66-7.58 (m, 2H), 7.56 (d, J=8.1 Hz, 1H), 7.52-7.42 (m, 3H), 7.26 (d, J=7.1 Hz, 1H), 7.16-7.06 (m, 5H), 7.03 (t, J=4.4 Hz, 1H), 5.07 (dd, J=12.8, 5.4 Hz, 1H), 4.22 (d, J=4.2 Hz, 2H), 4.13 (t, J=6.0 Hz, 2H), 3.90-3.53 (m, 4H), 3.04-2.83 (m, 6H), 2.70 (s, 3H), 2.62-2.51 (m, 3H), 2.10-2.00 (m, 3H).

### Example 52. 2-((4-(3-(4-(5-(2-(2,6-dioxopiperidin-3-yl)-1-oxoisoindolin-4-yl)pentyl)piperazin-1-yl)propoxy)phenyl)sulfonamido)-4-(4-methylnaphthalen-1-yl)benzoic acid (259)

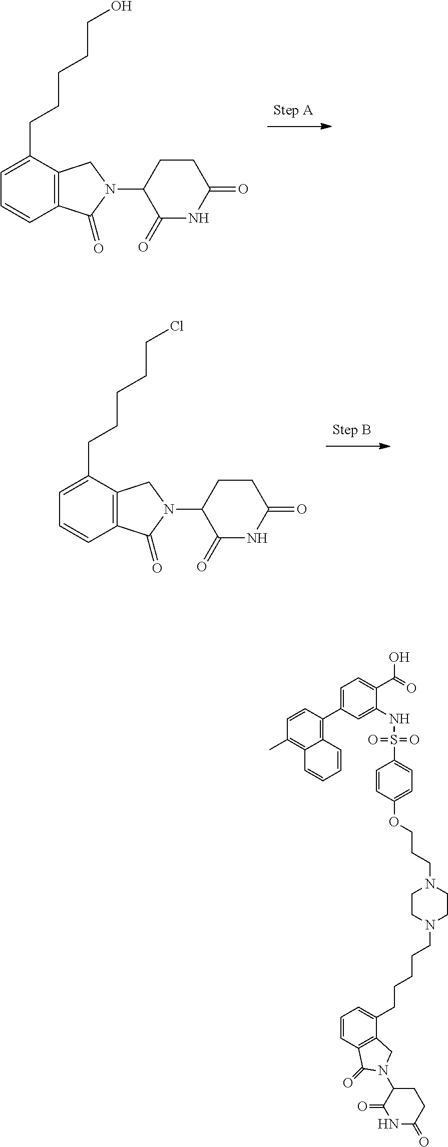 [SMILES: O=C1CCC(N2Cc3c(CCCCCO)cccc3C2=O)C(=O)N1 ; O=C1CCC(N2Cc3c(CCCCCCl)cccc3C2=O)C(=O)N1 ; Cc1ccc(-c2ccc(C(=O)O)c(NS(=O)(=O)c3ccc(OCCCN4CCN(CCCCCc5cccc6c5CN(C5CCC(=O)NC5=O)C6=O)CC4)cc3)c2)c2ccccc12]

### Step A

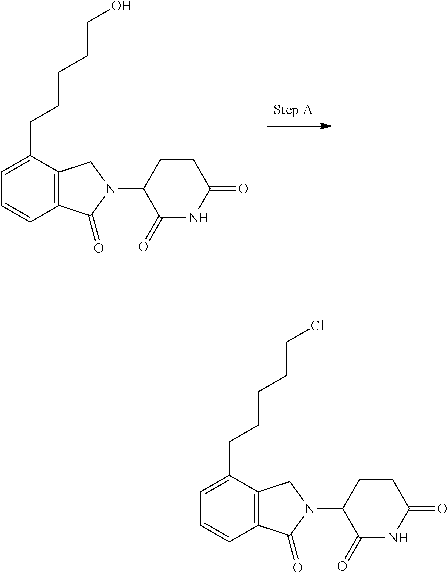 [SMILES: O=C1CCC(N2Cc3c(CCCCCO)cccc3C2=O)C(=O)N1 ; O=C1CCC(N2Cc3c(CCCCCCl)cccc3C2=O)C(=O)N1]

To a suspension of 3-(4-(5-hydroxypentyl)-1-oxoisoindolin-2-yl)piperidine-2,6-dione (1.65 g, 5 mmol) in DCM (60 mL) was added thionyl chloride (0.72 g, 6 mmol). The mixture was heated to 60° C. for 18 hours, giving a clear orange liquid. The mixture was cooled and concentrated under argon atmosphere giving a thick solid precipitate which was filtered, washed with ethyl ether and dried in vacuo, to give 3-(4-(5-chloropentyl)-1-oxoisoindolin-2-yl)piperidine-2,6-dione as a beige solid (0.842 g, 2.4 mmol, 48%).

LCMS (ESI+): m/z 349.2 [M+H]⁺

### Step B

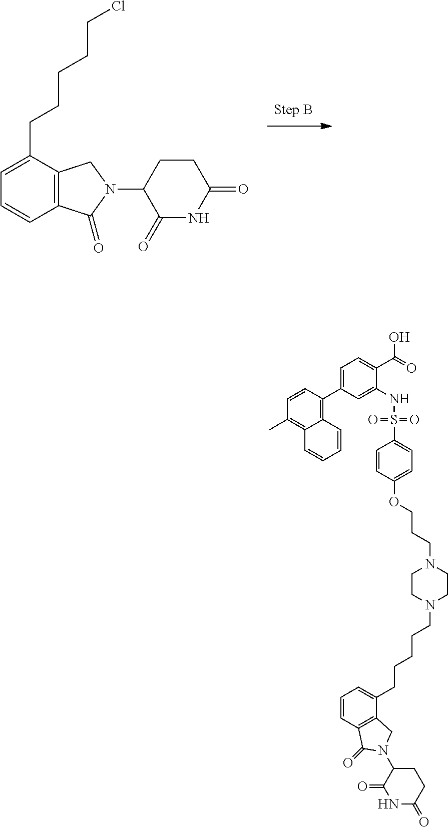 [SMILES: O=C1CCC(N2Cc3c(CCCCCCl)cccc3C2=O)C(=O)N1 ; Cc1ccc(-c2ccc(C(=O)O)c(NS(=O)(=O)c3ccc(OCCCN4CCN(CCCCCc5cccc6c5CN(C5CCC(=O)NC5=O)C6=O)CC4)cc3)c2)c2ccccc12]

4-(4-methylnaphthalen-1-yl)-2-((4-(3-(piperazin-1-yl)propoxy)phenyl)sulfonamido)benzoic acid dihydrochloride (25.0 mg, 0.045 mmol), 3-(4-(5-chloropentyl)-1-oxoisoindolin-2-yl)piperidine-2,6-dione (15.6 mg, 0.045 mmol), KI (7.4 mg, 0.045 mmol) and KHCO₃ (17.9 mg, 0.179 mmol) were dissolved in DMF (0.500 mL), the mixture warmed to 60° C. and stirred for 2 h. After that time LCMS second portion of 3-(4-(5-chloropentyl)-1-oxoisoindolin-2-yl)piperidine-2,6-dione (7.0 mg, 0.020 mmol) was added and the mixture stirred at 60° C. for 1.5 h, whereupon LCMS indicated full consumption of the starting material. The reaction mixture was concentrated in vacuo and purified by flash column chromatography (SiO2, MeOH:DCM, 10-30%) to give 2-((4-(3-(4-(5-(2-(2,6-dioxopiperidin-3-yl)-1-oxoisoindolin-4-yl)pentyl)piperazin-1-yl)propoxy)phenyl)sulfonamido)-4-(4-methylnaphthalen-1-yl)benzoic acid (23.6 mg, 0.027 mmol, 61%) as a yellow solid.

LCMS (ESI+): m/z 872.9 [M+H]⁺

¹H NMR (500 MHz, DMSO) δ 10.99 (s, 1H), 8.11 (d, J=8.5 Hz, 1H), 7.99 (d, J=8.0 Hz, 1H), 7.70 (d, J=8.8 Hz, 2H), 7.66-7.52 (m, 3H), 7.52-7.38 (m, 5H), 7.25 (d, J=7.1 Hz, 1H), 7.06 (d, J=8.9 Hz, 3H), 5.14 (dd, J=13.3, 5.1 Hz, 1H), 4.46 (d, J=17.1 Hz, 1H), 4.30 (d, J=17.1 Hz, 1H), 4.09 (t, J=6.1 Hz, 2H), 3.48-3.31 (m, 6H), 2.98-2.87 (m, 1H), 2.70 (s, 3H), 2.68-2.57 (m, 3H), 2.54-2.51 (m, 1H), 2.47-2.33 (m, 2H), 2.06-1.98 (m, 1H), 1.98-1.86 (m, 2H), 1.71-1.53 (m, 4H), 1.37-1.24 (m, 6H).

### Example 53. 6-chloro-1-(2-(4-(2-((2-(2,6-dioxopiperidin-3-yl)-1,3-dioxoisoindolin-4-yl)oxy)acetyl)piperazin-1-yl)ethyl)-3-(3-((3-methoxynaphthalen-1-yl)oxy)propyl)-7-(1,3,5-trimethyl-1H-pyrazol-4-yl)-1H-indole-2-carboxylic acid (218)

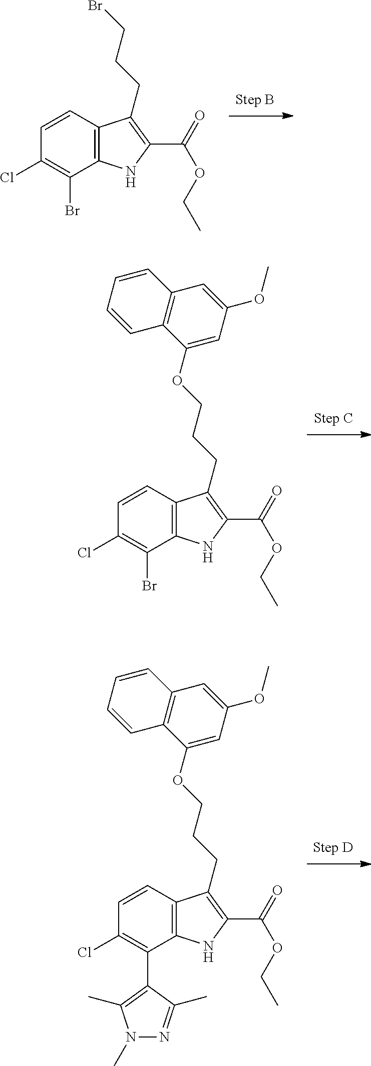

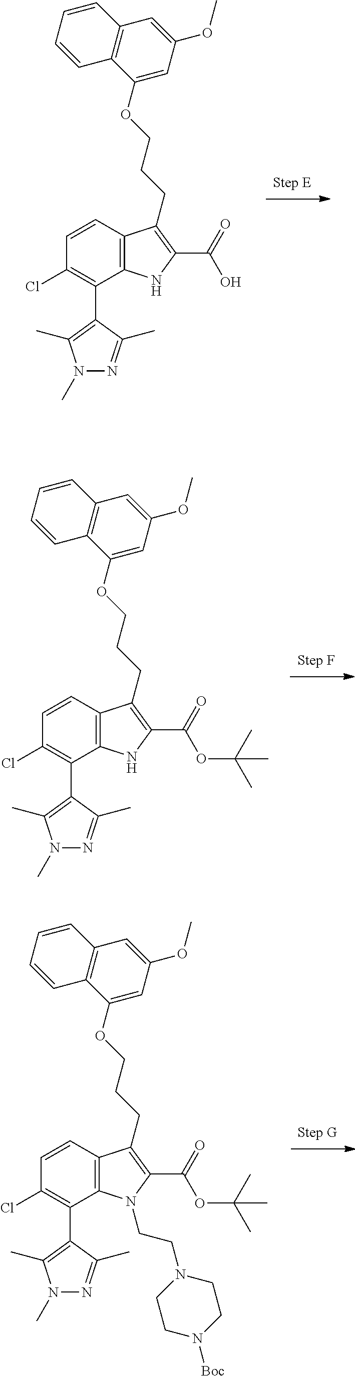

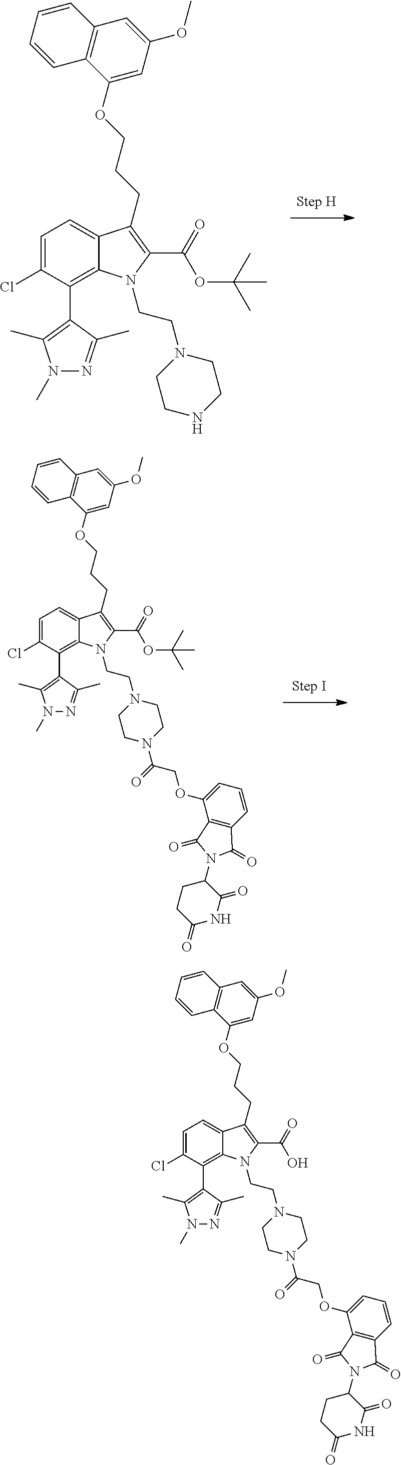

### Step A

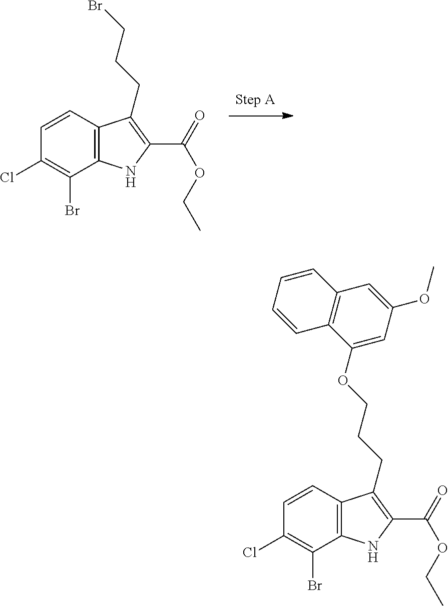 [SMILES: CCOC(=O)c1[nH]c2c(Br)c(Cl)ccc2c1CCCBr ; CCOC(=O)c1[nH]c2c(Br)c(Cl)ccc2c1CCCOc1cc(OC)cc2ccccc12]

To a well stirred solution of crude ethyl 7-bromo-3-(3-bromopropyl)-6-chloro-1H-indole-2-carboxylate (3 g, 7.09 mmol) in DMF (35 mL) was added 3-methoxynaphthalen-1-ol, (0.987 g, 5.674 mmol), KI (1.2 g, 7.096 mmol), Cs₂CO₃ (6.95 g, 21.27 mmol) successively at RT under nitrogen. The reaction mixture was allowed to stir at 60° C. for 16 h. After complete consumption of the starting material (monitored by TLC and LCMS) the reaction mixture was diluted with EtOAc and washed successively with water and brine. The organic layer was dried over Na₂SO₄ and evaporated under reduced pressure to get the crude compound, which was then purified by triturating with Et₂O to get 2 g (3.875 mmol, 45% over two steps) of as off white solid.

### Step B

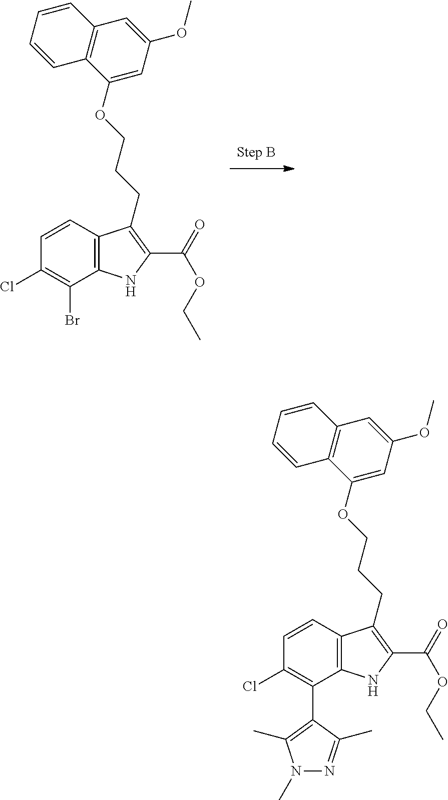 [SMILES: CCOC(=O)c1[nH]c2c(Br)c(Cl)ccc2c1CCCOc1cc(OC)cc2ccccc12 ; CCOC(=O)c1[nH]c2c(-c3c(C)nn(C)c3C)c(Cl)ccc2c1CCCOc1cc(OC)cc2ccccc12]

To a stirred solution of ethyl 7-bromo-6-chloro-3-(3-((3-methoxynaphthalen-1-yl)oxy)propyl)-1H-indole-2-carboxylate (1.5 g, 2.907 mmol) in dioxane (15 mL) and water (3 mL) were added 1,3,5-trimethyl-4-(4,4,5,5-tetramethyl-1,3,2-dioxaborolan-2-yl)-1H-pyrazole (2.058 g, 8.72 mmol) and K₂CO₃ (1.60 g, 11.62 mmol) successively at RT. The mixture was deoxygenated with argon and to it was added Pd(dppf)Cl₂ (0.319 g, 0.436 mmol) under argon atmosphere. Then the reaction mixture was heated under reflux for 16 h. After complete consumption of the starting material (monitored by TLC and LCMS) the reaction mixture was filtered through celite pad and the filtrate was evaporated under reduced pressure get the crude material. It was then diluted with EtOAc and washed successively with water and brine The organic layer was dried over Na₂SO₄ and evaporated under reduced pressure to get the crude compound, which was then purified by column chromatography (SiO2, 20% EtOAc in DCM) to get 1.2 g (2.2 mmol, 75%) of ethyl 6-chloro-3-(3-((3-methoxynaphthalen-1-yl)oxy)propyl)-7-(1,3,5-trimethyl-1H-pyrazol-4-yl)-1H-indole-2-carboxylate as brown solid.

LCMS (ESI+): m/z 545.9 [M+H]⁺

### Step C

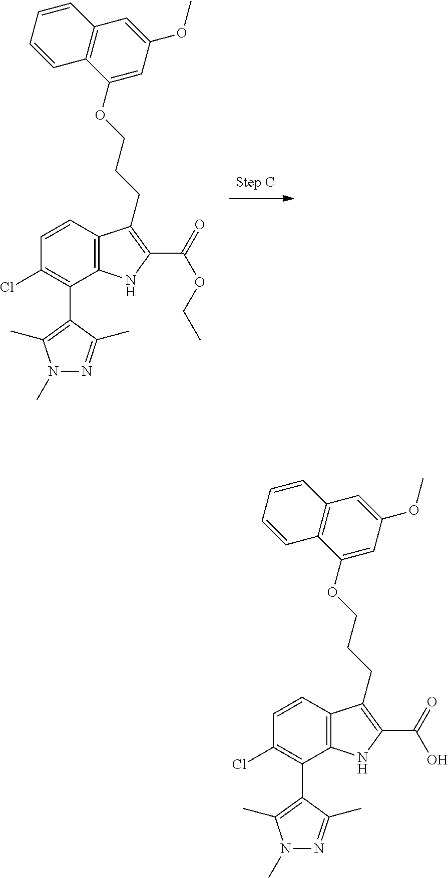 [SMILES: CCOC(=O)c1[nH]c2c(-c3c(C)nn(C)c3C)c(Cl)ccc2c1CCCOc1cc(OC)cc2ccccc12 ; COc1cc(OCCCc2c(C(=O)O)[nH]c3c(-c4c(C)nn(C)c4C)c(Cl)ccc23)c2ccccc2c1]

Ethyl 6-chloro-3-(3-((3-methoxynaphthalen-1-yl)oxy)propyl)-7-(1,3,5-trimethyl-1H-pyrazol-4-yl)-1H-indole-2-carboxylate (1.2 g, 2.2 mmol) was dissolved in EtOH (20 mL) and a solution of NaOH (0.35 g, 8.8 mmol) in water (4 mL) was added to it. The mixture was heated under reflux for 16 h. After complete consumption of the starting material (monitored by TLC and LCMS) the reaction mixture was cooled down to room temperature, solvents were evaporated under reduced pressure to get the crude reaction mixture. It was then diluted with water and extracted with EtOAc. Aqueous layer was carefully acidified using 1M HCl to maintain pH=3, extracted with EtOAc (3×50 ml), dried over Na₂SO₄, filtered, and concentrated in vacuum to afford 0.9 g (1.73 mmol, 79%) of 6-chloro-3-(3-((3-methoxynaphthalen-1-yl)oxy)propyl)-7-(1,3,5-trimethyl-1H-pyrazol-4-yl)-1H-indole-2-carboxylic acid as off white solid

LCMS (ESI+): m/z 518.0 [M+H]⁺

### Step D

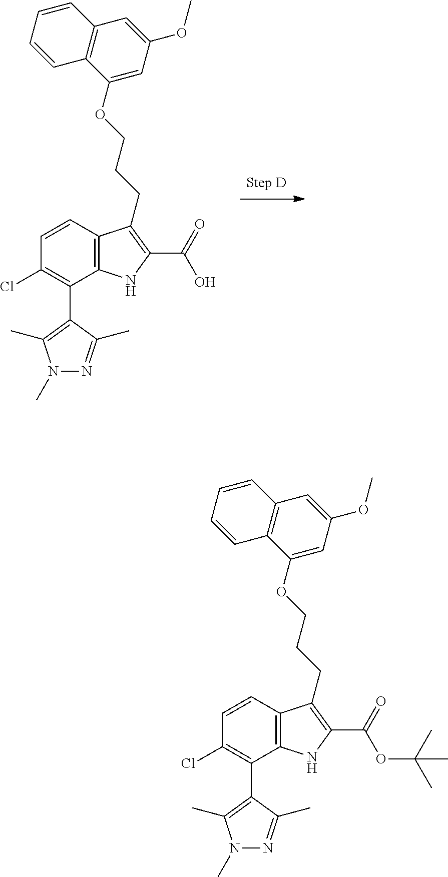 [SMILES: COc1cc(OCCCc2c(C(=O)O)[nH]c3c(-c4c(C)nn(C)c4C)c(Cl)ccc23)c2ccccc2c1 ; COc1cc(OCCCc2c(C(=O)OC(C)(C)C)[nH]c3c(-c4c(C)nn(C)c4C)c(Cl)ccc23)c2ccccc2c1]

6-chloro-3-(3-((3-methoxynaphthalen-1-yl)oxy)propyl)-7-(1,3,5-trimethyl-1H-pyrazol-4-yl)-1H-indole-2-carboxylic acid (0.9 g, 1.73 mmol,) was suspended in toluene (9 mL) and the mixture was heated to reflux under nitrogen. N,N-dimethylformamide di-tert-butyl acetal (5 ml, 20.85 mmol) was added dropwise to the refluxing mixture. The mixture was heated under reflux for 16 h under nitrogen. After complete consumption of the starting material (monitored by TLC and LCMS) reaction mixture was then diluted with EtOAc, washed successively with NaHCO₃ (sat.), water and brine. Organic layer was dried over Na₂SO₄ and evaporated under reduced pressure to get the crude compound, which was then purified by column chromatography (SiO2, 30% EtOAc in DCM) to get tert-butyl 6-chloro-3-(3-((3-methoxynaphthalen-1-yl)oxy)propyl)-7-(1,3,5-trimethyl-1H-pyrazol-4-yl)-1H-indole-2-carboxylate (700 mg, 1.22 mmol, 70%) as off white solid.

LCMS (ESI+): m/z 574.4 [M+H]⁺

### Step E

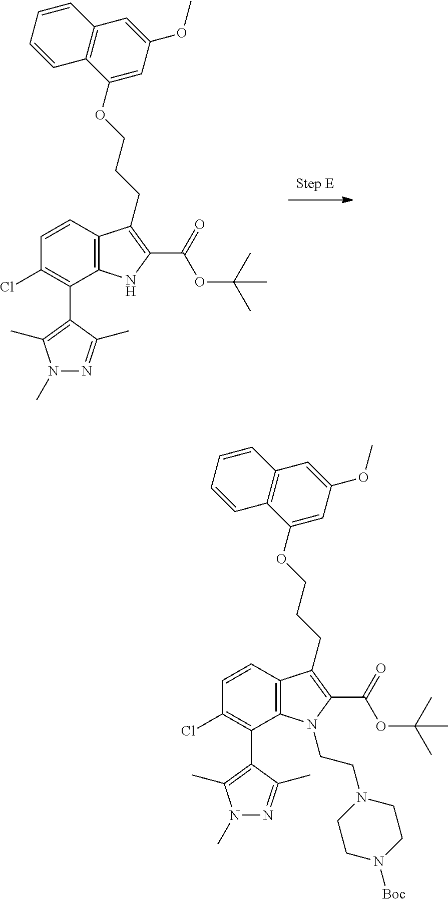 [SMILES: COc1cc(OCCCc2c(C(=O)OC(C)(C)C)[nH]c3c(-c4c(C)nn(C)c4C)c(Cl)ccc23)c2ccccc2c1 ; COc1cc(OCCCc2c(C(=O)OC(C)(C)C)n(CCN3CCN(C(=O)OC(C)(C)C)CC3)c3c(-c4c(C)nn(C)c4C)c(Cl)ccc23)c2ccccc2c1]

To a well stirred solution of tert-butyl 6-chloro-3-(3-((3-methoxynaphthalen-1-yl)oxy)propyl)-7-(1,3,5-trimethyl-1H-pyrazol-4-yl)-1H-indole-2-carboxylate (0.70 g, 1.22 mmol) in DMF (6 mL) was added tert-butyl 4-(2-chloroethyl)piperazine-1-carboxylate (0.606 g, 2.44 mmol) followed by Cs₂CO₃ (1.98 g, 6.098 mmol) at RT under nitrogen. The reaction mixture was stirred at 90° C. for 16 h under nitrogen. After complete consumption of the starting material (monitored by TLC and LCMS) the reaction mixture was diluted with EtOAc and washed successively with water and brine. The organic layer was dried over Na₂SO₄ and evaporated under reduced pressure to get the crude compound, which was then purified by column chromatography (SiO₂, 50% EtOAc in hexane) to get tert-butyl 1-(2-(4-(tert-butoxycarbonyl)piperazin-1-yl)ethyl)-6-chloro-3-(3-((3-methoxynaphthalen-1-yl)oxy)propyl)-7-(1,3,5-trimethyl-1H-pyrazol-4-yl)-1H-indole-2-carboxylate (650 mg, 0.827 mmol, 67.7%) as off white solid.

LCMS (ESI+): m/z 786.4 [M+H]⁺

### Step F

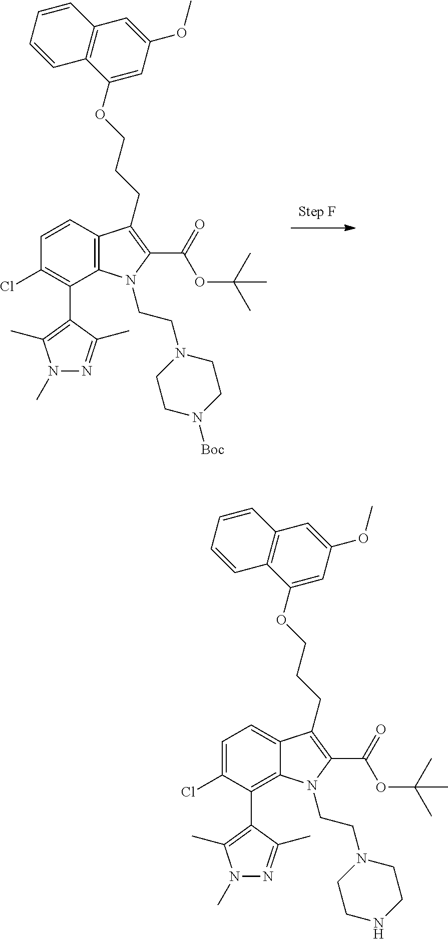 [SMILES: COc1cc(OCCCc2c(C(=O)OC(C)(C)C)n(CCN3CCN(C(=O)OC(C)(C)C)CC3)c3c(-c4c(C)nn(C)c4C)c(Cl)ccc23)c2ccccc2c1 ; COc1cc(OCCCc2c(C(=O)OC(C)(C)C)n(CCN3CCNCC3)c3c(-c4c(C)nn(C)c4C)c(Cl)ccc23)c2ccccc2c1]

tert-butyl 1-(2-(4-(tert-butoxycarbonyl)piperazin-1-yl)ethyl)-6-chloro-3-(3-((3-methoxynaphthalen-1-yl)oxy)propyl)-7-(1,3,5-trimethyl-1H-pyrazol-4-yl)-1H-indole-2-carboxylate (0.65 g, 0.827 mmol) was dissolved in 4M HCl in dioxane (20 mL) at 0° C. and the mixture was stirred for 2 h under nitrogen at same temperature. After complete consumption of the starting material (monitored by LCMS) to the reaction mixture was added drop wise cold 1M NaOH solution at 0° C. to maintain the pH˜5 to 6. The aqueous layer was extracted 2-3 times with DCM. The combined organics were dried over Na₂SO₄ and concentrated in vacuo to get the crude compound which was purified by column chromatography (amine SiO₂, MeOH:DCM, 5-10%) to get tert-butyl 6-chloro-3-(3-((3-methoxynaphthalen-1-yl)oxy)propyl)-1-(2-(piperazin-1-yl)ethyl)-7-(1,3,5-trimethyl-1H-pyrazol-4-yl)-1H-indole-2-carboxylate (360 mg, 0.52 mmol, 63%) as off white solid.

LCMS (ESI+): m/z 686.4 [M+H]⁺

### Step G

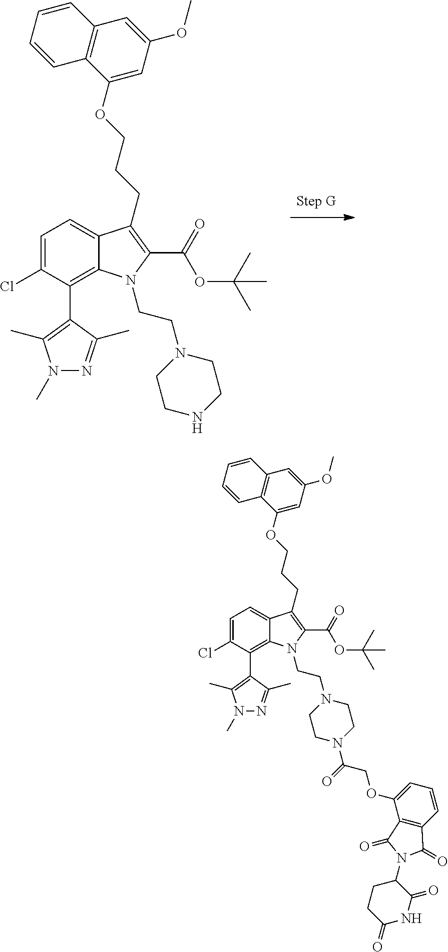 [SMILES: COc1cc(OCCCc2c(C(=O)OC(C)(C)C)n(CCN3CCNCC3)c3c(-c4c(C)nn(C)c4C)c(Cl)ccc23)c2ccccc2c1 ; COc1cc(OCCCc2c(C(=O)OC(C)(C)C)n(CCN3CCN(C(=O)COc4cccc5c4C(=O)N(C4CCC(=O)NC4=O)C5=O)CC3)c3c(-c4c(C)nn(C)c4C)c(Cl)ccc23)c2ccccc2c1]

Tert-butyl 6-chloro-3-{3-[(3-methoxynaphthalen-1-yl)oxy]propyl}-1-[2-(piperazin-1-yl)ethyl]-7-(1,3,5-trimethyl-1H-pyrazol-4-yl)-1H-indole-2-carboxylate (50.0 mg, 0.073 mmol), 2-{[2-(2,6-dioxopiperidin-3-yl)-1,3-dioxo-2,3-dihydro-1H-isoindol-4-yl]oxy}acetic acid (29.0 mg, 0.087 mmol) and HATU (33.2 mg, 0.087 mmol) were dissolved in dry DMF (2.0 mL) and to the mixture was added DIPEA (0.063 mL, 0.364 mmol). The reaction was stirred at RT for 2 h. The solvent was evaporated, resulting residues were dissolved in DCM and washed with brine. The organic layer was dried over Na₂SO₄, filtered and concentrated in vacuo. The crude tert-butyl 6-chloro-1-(2-(4-(2-((2-(2,6-dioxopiperidin-3-yl)-1,3-dioxoisoindolin-4-yl)oxy)acetyl)piperazin-1-yl)ethyl)-3-(3-((3-methoxynaphthalen-1-yl)oxy)propyl)-7-(1,3,5-trimethyl-1H-pyrazol-4-yl)-1H-indole-2-carboxylate (85.0 mg, crude) was used in the next step without further purification.

LCMS (ESI+): m/z 1000.3 [M+H]⁺

### Step H

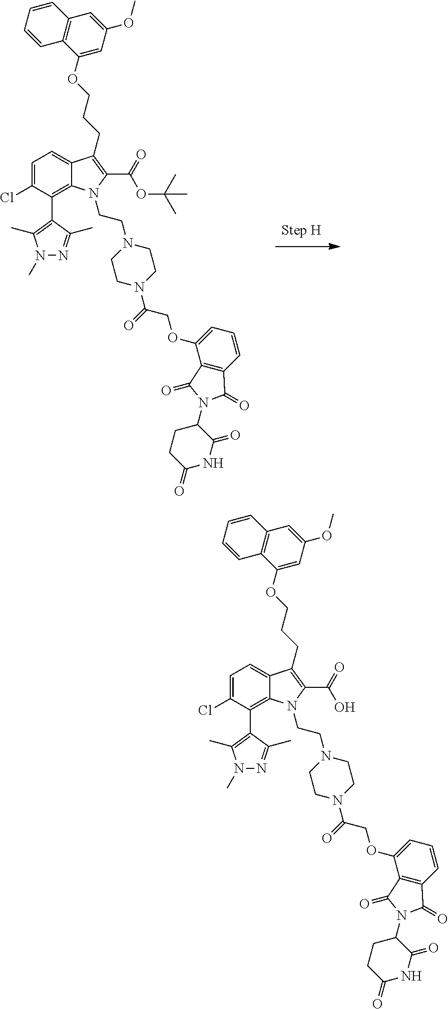 [SMILES: COc1cc(OCCCc2c(C(=O)OC(C)(C)C)n(CCN3CCN(C(=O)COc4cccc5c4C(=O)N(C4CCC(=O)NC4=O)C5=O)CC3)c3c(-c4c(C)nn(C)c4C)c(Cl)ccc23)c2ccccc2c1 ; COc1cc(OCCCc2c(C(=O)O)n(CCN3CCN(C(=O)COc4cccc5c4C(=O)N(C4CCC(=O)NC4=O)C5=O)CC3)c3c(-c4c(C)nn(C)c4C)c(Cl)ccc23)c2ccccc2c1]

To tert-butyl 6-chloro-1-(2-(4-(2-((2-(2,6-dioxopiperidin-3-yl)-1,3-dioxoisoindolin-4-yl)oxy)acetyl)piperazin-1-yl)ethyl)-3-(3-((3-methoxynaphthalen-1-yl)oxy)propyl)-7-(1,3,5-trimethyl-1H-pyrazol-4-yl)-1H-indole-2-carboxylate (85.0 mg, crude) a product in DCM (1.0 mL) was added TFA (1.0 mL, 13.059 mmol) and the mixture was stirred for 18 h at RT. After the reaction was completed (monitored by LCMS), the crude mixture was concentrated in vacuo and purified by preparative HPLC (H₂O:MeCN+0.1% FA). The isolated product, 6-chloro-1-(2-(4-(2-((2-(2,6-dioxopiperidin-3-yl)-1,3-dioxoisoindolin-4-yl)oxy)acetyl)piperazin-1-yl)ethyl)-3-(3-((3-methoxynaphthalen-1-yl)oxy)propyl)-7-(1,3,5-trimethyl-1H-pyrazol-4-yl)-1H-indole-2-carboxylic acid (41.6 mg, 0.044 mmol, 60% over 2 steps) was obtained as light yellow solid.

LCMS (ESI+): m/z 944.3 [M+H]⁺

¹H NMR (500 MHz, DMSO) δ 13.07 (bs, J=333.2 Hz, 1H), 11.09 (s, 1H), 8.06 (d, J=8.3 Hz, 1H), 7.78-7.72 (m, 3H), 7.49-7.42 (m, 2H), 7.33-7.26 (m, 2H), 7.22 (d, J=8.5 Hz, 1H), 6.87 (d, J=2.1 Hz, 1H), 6.52 (d, J=2.2 Hz, 1H), 5.13-5.06 (m, 3H), 4.32-4.25 (m, 1H), 4.17 (t, J=6.1 Hz, 3H), 3.84 (s, 3H), 3.75 (s, 3H), 3.29-3.23 (m, 4H), 2.88 (ddd, J=16.9, 13.7, 5.4 Hz, 1H), 2.62-2.52 (m, 2H), 2.48-2.46 (m, 1H), 2.20 (p, J=6.5 Hz, 2H), 2.16-1.96 (m, 11H), 1.87 (s, 3H).

### Example 54. 6-chloro-1-(2-(4-(2-((2-(2,6-dioxopiperidin-3-yl)-1-oxoisoindolin-4-yl)oxy)acetyl)piperazin-1-yl)ethyl)-3-(3-((5,6,7,8-tetrahydronaphthalen-1-yl)oxy)propyl)-7-(1,3,5-trimethyl-1H-pyrazol-4-yl)-1H-indole-2-carboxylic acid (219)

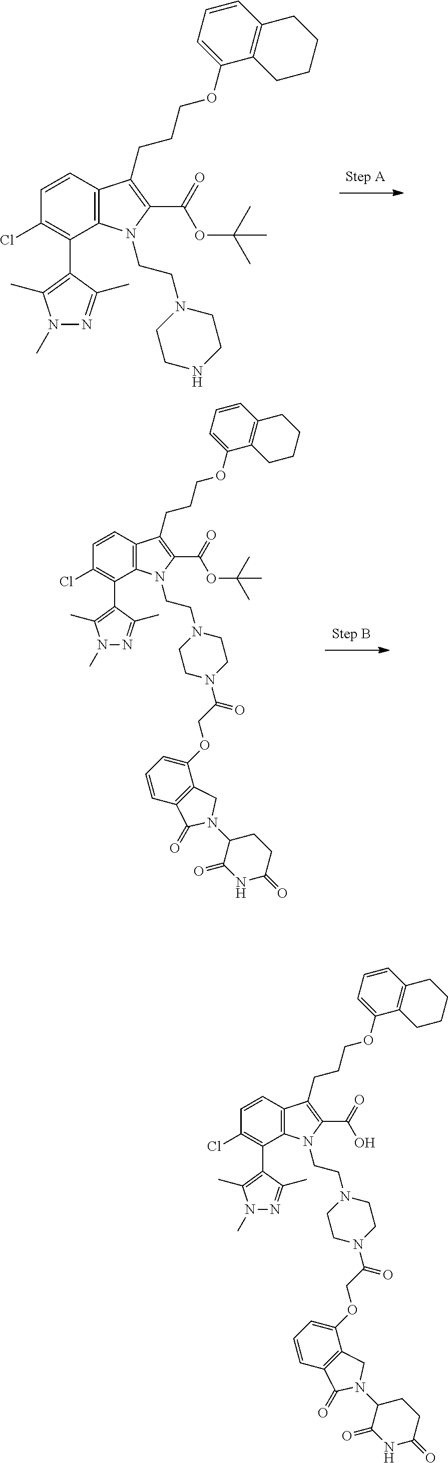 [SMILES: Cc1nn(C)c(C)c1-c1c(Cl)ccc2c(CCCOc3cccc4c3CCCC4)c(C(=O)OC(C)(C)C)n(CCN3CCNCC3)c12 ; Cc1nn(C)c(C)c1-c1c(Cl)ccc2c(CCCOc3cccc4c3CCCC4)c(C(=O)OC(C)(C)C)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4c3CCCC4)c(C(=O)O)n(CCN3CCN(C(=O)COc4cccc5c4CN(C4CCC(=O)NC4=O)C5=O)CC3)c12]

### Step A

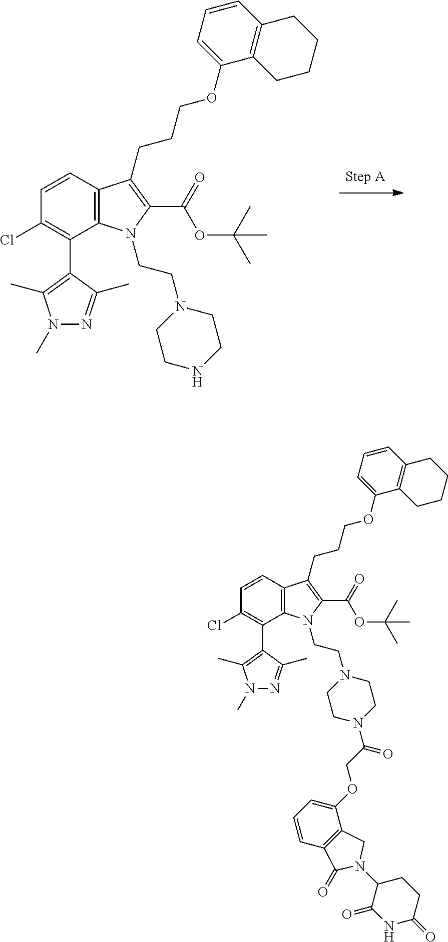 [SMILES: Cc1nn(C)c(C)c1-c1c(Cl)ccc2c(CCCOc3cccc4c3CCCC4)c(C(=O)OC(C)(C)C)n(CCN3CCNCC3)c12 ; Cc1nn(C)c(C)c1-c1c(Cl)ccc2c(CCCOc3cccc4c3CCCC4)c(C(=O)OC(C)(C)C)n(CCN3CCN(C(=O)COc4cccc5c4CN(C4CCC(=O)NC4=O)C5=O)CC3)c12]

2-{[2-(2,6-dioxopiperidin-3-yl)-1-oxo-2,3-dihydro-1H-isoindol-4-yl]oxy}acetic acid (26.5 mg, 0.083 mmol) was dissolved in dry DMF (1.5 mL) under argon atmosphere and DIPEA (0.040 mL, 0.227 mmol), followed by tert-butyl 6-chloro-1-(2-(piperazin-1-yl)ethyl)-3-(3-((5,6,7,8-tetrahydronaphthalen-1-yl)oxy)propyl)-7-(1,3,5-trimethyl-1H-pyrazol-4-yl)-1H-indole-2-carboxylate (50.0 mg, 0.076 mmol) was added. Reaction mixture was cooled down to 0° C. and HATU (34.6 mg, 0.091 mmol) was added as a solution in dry DMF (0.2 mL). The reaction was stirred under argon at RT for 20 min. The solution was diluted with DCM and washed with brine, NaHCO₃ (2×), again brine and water. Organic layer was dried over anhydrous MgSO₄, filtered, concentrated and dried under reduced pressure to give tert-butyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((5,6,7,8-tetrahydronaphthalen-1-yl)oxy)propyl)-7-(1,3,5-trimethyl-1H-pyrazol-4-yl)-1H-indole-2-carboxylate (70.7 mg, crude) as orange oil. The product was used in the next step without further purification.

LCMS (ESI+): m/z 960.2 [M+H]⁺

### Step B

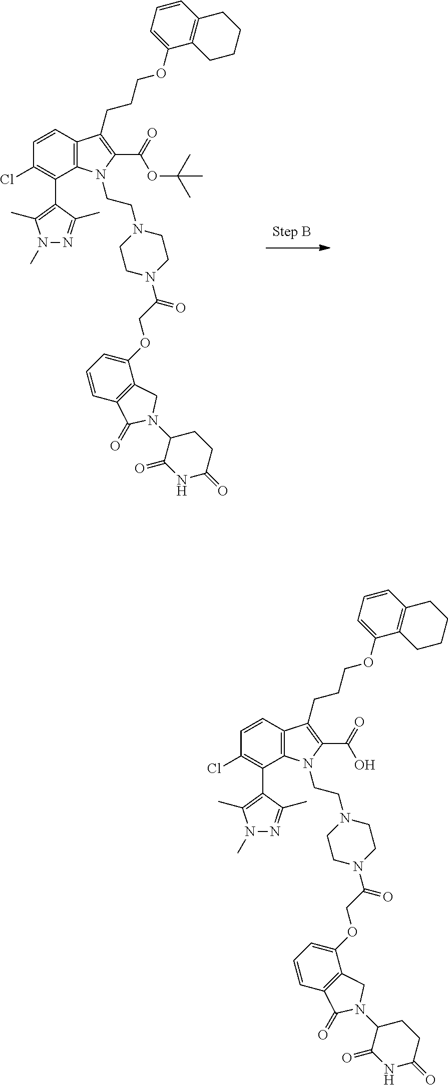 [SMILES: Cc1nn(C)c(C)c1-c1c(Cl)ccc2c(CCCOc3cccc4c3CCCC4)c(C(=O)OC(C)(C)C)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4c3CCCC4)c(C(=O)O)n(CCN3CCN(C(=O)COc4cccc5c4CN(C4CCC(=O)NC4=O)C5=O)CC3)c12]

Tert-butyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((5,6,7,8-tetrahydronaphthalen-1-yl)oxy)propyl)-7-(1,3,5-trimethyl-1H-pyrazol-4-yl)-1H-indole-2-carboxylate (70.7 mg, crude) was dissolved in dry DCM (0.56 mL) under argon atmosphere. TFA (0.56 mL, 7.358 mmol) was added and the reaction was stirred for 18 h at RT. After that time LCMS indicated complete consumption of the starting material. DCM and TFA were evaporated under reduced pressure and the resulting residue dissolved in DMSO (4 mL). The solution was passed through a syringe filter and purified by preparative HPLC (H₂O:MeCN+0.1% FA) to give 6-chloro-1-(2-(4-(2-((2-(2,6-dioxopiperidin-3-yl)-1-oxoisoindolin-4-yl)oxy)acetyl)piperazin-1-yl)ethyl)-3-(3-((5,6,7,8-tetrahydronaphthalen-1-yl)oxy)propyl)-7-(1,3,5-trimethyl-1H-pyrazol-4-yl)-1H-indole-2-carboxylic acid (16.7 mg, 0.018 mmol, 24% over two steps) as white solid.

LCMS (ESI+): m/z 903.7 [M+H]⁺

¹H NMR (500 MHz, DMSO) δ 13.35 (s, 1H), 10.98 (s, 1H), 7.71 (d, J=8.5 Hz, 1H), 7.46 (t, J=7.8 Hz, 1H), 7.33 (d, J=7.5 Hz, 1H), 7.27 (d, J=8.5 Hz, 1H), 7.13 (d, J=8.2 Hz, 1H), 7.00 (t, J=7.9 Hz, 1H), 6.65 (t, J=7.5 Hz, 2H), 5.12 (dd, J=13.3, 5.1 Hz, 1H), 4.97 (s, 2H), 4.40 (d, J=17.3 Hz, 1H), 4.27 (d, J=17.4 Hz, 2H), 4.22-4.14 (m, 1H), 4.00 (t, J=6.3 Hz, 2H), 3.77 (d, J=1.8 Hz, 3H), 3.38-3.36 (m, 1H), 3.30-3.28 (m, 1H), 3.18 (t, J=7.5 Hz, 2H), 2.93 (ddd, J=17.2, 13.5, 5.3 Hz, 1H), 2.69 (t, J=6.1 Hz, 2H), 2.64-2.59 (m, 3H), 2.57-2.54 (m, 2H), 2.48-2.41 (m, 1H), 2.17-2.04 (m, 8H), 2.03 (s, 3H), 2.02-1.98 (m, 1H), 1.90 (s, 3H), 1.80-1.65 (m, 4H).

### Example 55. 6-chloro-1-{2-[4-(2-{[2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (220)

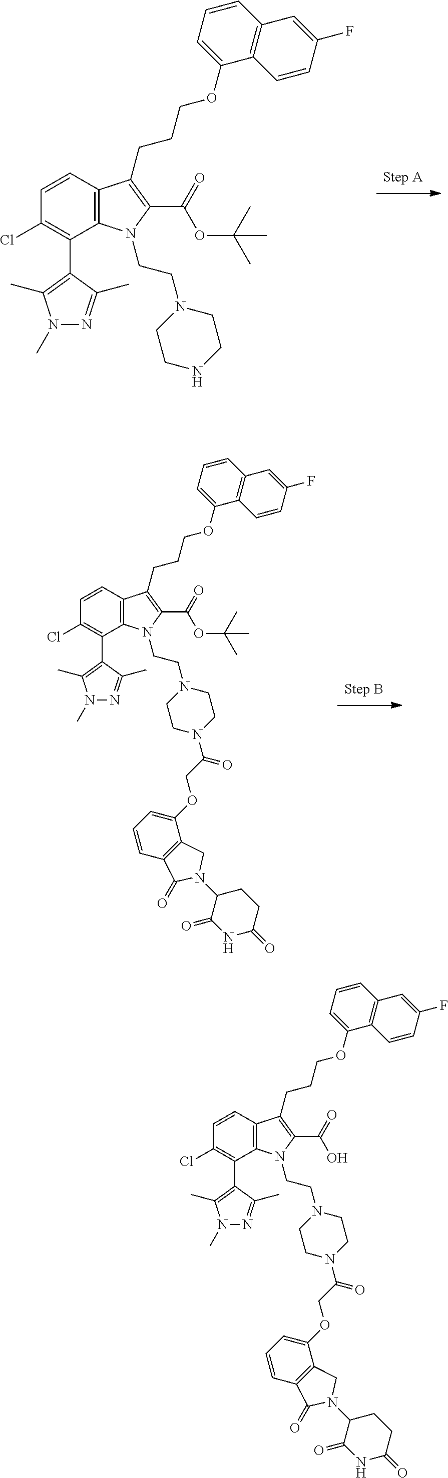 [SMILES: Cc1nn(C)c(C)c1-c1c(Cl)ccc2c(CCCOc3cccc4cc(F)ccc34)c(C(=O)OC(C)(C)C)n(CCN3CCNCC3)c12 ; Cc1nn(C)c(C)c1-c1c(Cl)ccc2c(CCCOc3cccc4cc(F)ccc34)c(C(=O)OC(C)(C)C)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4cccc5c4CN(C4CCC(=O)NC4=O)C5=O)CC3)c12]

### Step A

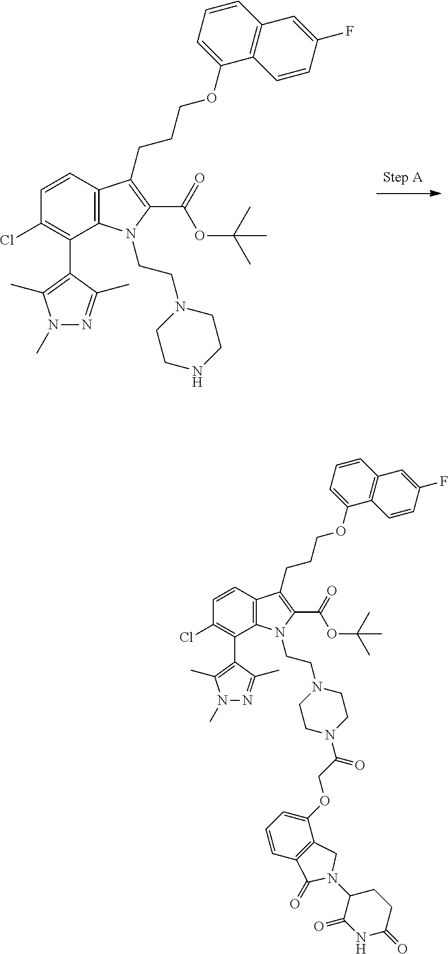 [SMILES: Cc1nn(C)c(C)c1-c1c(Cl)ccc2c(CCCOc3cccc4cc(F)ccc34)c(C(=O)OC(C)(C)C)n(CCN3CCNCC3)c12 ; Cc1nn(C)c(C)c1-c1c(Cl)ccc2c(CCCOc3cccc4cc(F)ccc34)c(C(=O)OC(C)(C)C)n(CCN3CCN(C(=O)COc4cccc5c4CN(C4CCC(=O)NC4=O)C5=O)CC3)c12]

2-{[2-(2,6-Dioxopiperidin-3-yl)-1-oxo-2,3-dihydro-1H-isoindol-4-yl]oxy}acetic acid (18.3 mg, 0.058 mmol) was dissolved in dry DMF (1.0 mL) under argon atmosphere and DIPEA (0.025 mL, 0.144 mmol) was added, followed by tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (32.3 mg, 0.048 mmol). The solution was cooled down to 0° C. and HATU (21.9 mg, 0.058 mmol) was added dropwise as a solution in 0.2 mL of dry DMF. The reaction was stirred under argon atmosphere, allowing to warm to room temperature. After 15 min full conversion was observed (monitored with LCMS). The solution was diluted with DCM and washed with brine, NaHCO₃ (sat.), again brine and water. Organic layer was dried over anhydrous MgSO₄, filtered, concentrated and dried under reduced pressure to give tert-butyl 6-chloro-1-{2-[4-(2-{[2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (44.7 mg, crude) as orange oil. The product was used in the next step without further purification.

LCMS (ESI+): 974.6 [M+H]⁺

### Step B

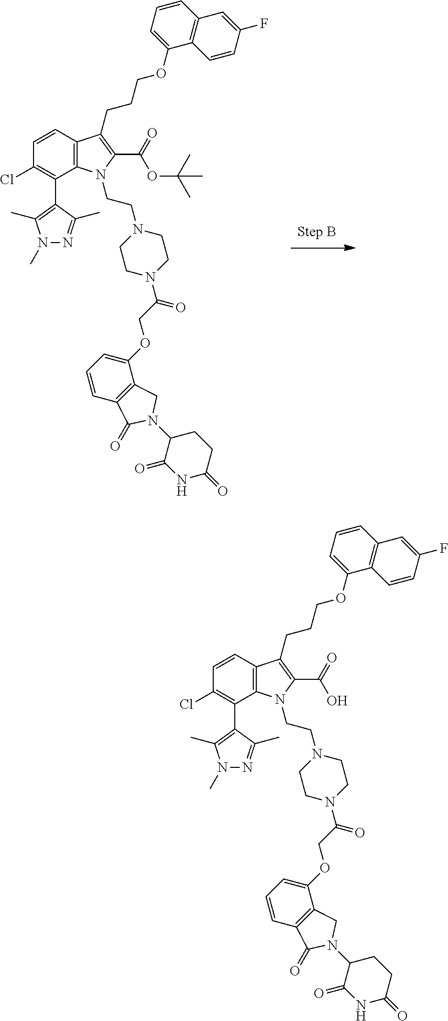 [SMILES: Cc1nn(C)c(C)c1-c1c(Cl)ccc2c(CCCOc3cccc4cc(F)ccc34)c(C(=O)OC(C)(C)C)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4cccc5c4CN(C4CCC(=O)NC4=O)C5=O)CC3)c12]

Tert-butyl 6-chloro-1-{2-[4-(2-{[2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (44.7 mg, crude) was dissolved in dry DCM (0.351 mL) under argon atmosphere. TFA (0.351 mL, 4.584 mmol) was added and the reaction was stirred for 18 h at room temperature. DCM and TFA were evaporated under reduced pressure and the resulting residue dissolved in DMSO and purified with preparative HPLC (H₂O:MeCN+0.1% FA) to give 6-chloro-1-{2-[4-(2-{[2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (12.6 mg, 0.014 mmol, 29% over two steps) as white solid.

LCMS (ESI+): m/z 917.6 [M+H]⁺

¹H NMR (500 MHz, DMSO, 353 K) δ 10.64 (s, 1H), 8.24 (dd, J=9.3, 5.9 Hz, 1H), 7.71 (d, J=8.5 Hz, 1H), 7.58 (dd, J=10.4, 2.6 Hz, 1H), 7.48-7.38 (m, 3H), 7.36-7.28 (m, 2H), 7.22 (d, J=8.5 Hz, 1H), 7.14 (d, J=8.1 Hz, 1H), 6.87 (dd, J=5.8, 2.8 Hz, 1H), 5.05 (dd, J=13.1, 5.2 Hz, 1H), 4.89 (s, 2H), 4.40 (d, J=17.1 Hz, 1H), 4.32 (d, J=17.2 Hz, 1H), 4.30-4.23 (m, 3H), 4.23-4.15 (m, 1H), 3.76 (d, J=1.8 Hz, 3H), 3.40-3.32 (m, 4H), 3.32-3.26 (m, 2H), 2.88 (ddd, J=17.2, 13.4, 5.5 Hz, 1H), 2.64-2.53 (m, 1H), 2.48-2.39 (m, 1H), 2.28-2.19 (m, 2H), 2.17-2.02 (m, 7H), 2.01 (s, 3H), 1.89 (s, 3H).

### Example 56. 6-chloro-1-{2-[4-(2-{[2-(2,6-dioxopiperidin-3-yl)-1-oxo-2,3-dihydro-1H-isoindol-4-yl]oxy}acetamido)piperidin-1-yl]ethyl}-3-[3-(naphthalen-1-yloxy)propyl]-7-(1,3,5-trimethyl-1H-pyrazol-4-yl)-1H-indole-2-carboxylic acid (221)

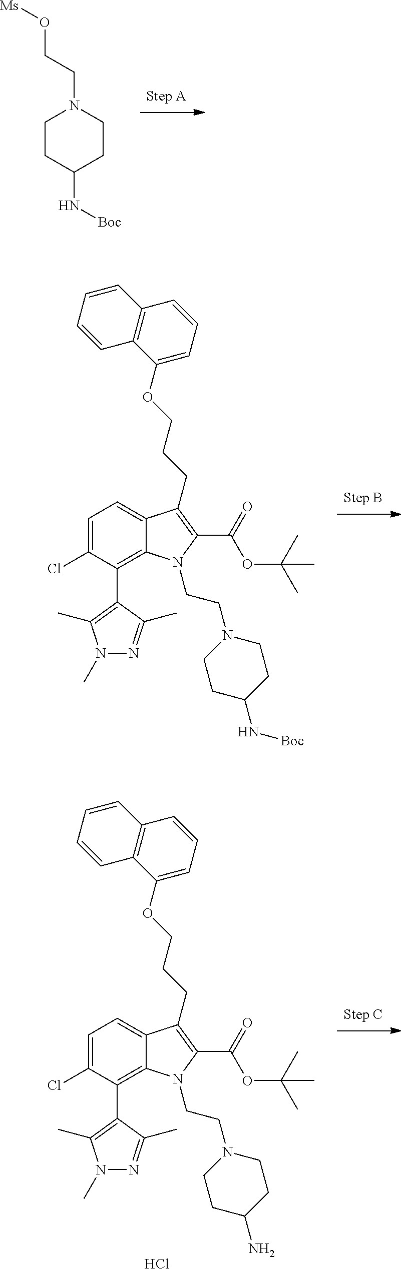

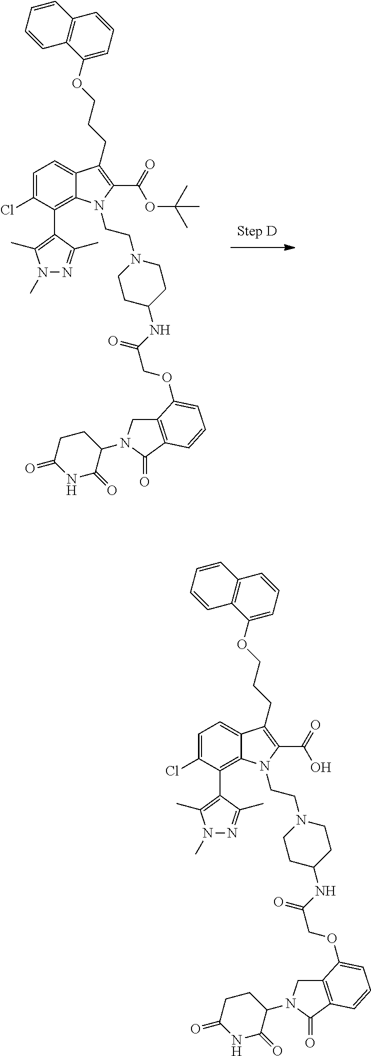 [SMILES: Cc1nn(C)c(C)c1-c1c(Cl)ccc2c(CCCOc3cccc4ccccc34)c(C(=O)OC(C)(C)C)n(CCN3CCC(NC(=O)COc4cccc5c4CN(C4CCC(=O)NC4=O)C5=O)CC3)c12 ; Cc1nn(C)c(C)c1-c1c(Cl)ccc2c(CCCOc3cccc4ccccc34)c(C(=O)O)n(CCN3CCC(NC(=O)COc4cccc5c4CN(C4CCC(=O)NC4=O)C5=O)CC3)c12]

### Step A

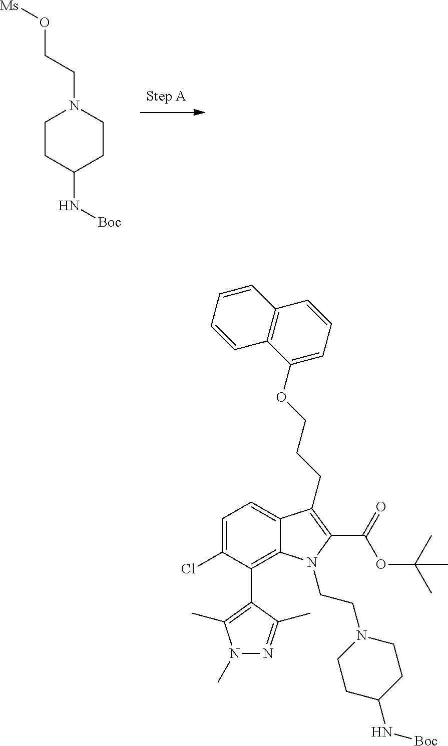 [SMILES: CC(C)(C)OC(=O)NC1CCN(CCOS(C)(=O)=O)CC1 ; Cc1nn(C)c(C)c1-c1c(Cl)ccc2c(CCCOc3cccc4ccccc34)c(C(=O)OC(C)(C)C)n(CCN3CCC(NC(=O)OC(C)(C)C)CC3)c12]

Tert-butyl N-{1-[2-(methanesulfonyloxy)ethyl]piperidin-4-yl}carbamate (40.0 mg, 0.124 mmol), tert-butyl 6-chloro-3-[3-(naphthalen-1-yloxy)propyl]-7-(1,3,5-trimethyl-1H-pyrazol-4-yl)-1H-indole-2-carboxylate (45.0 mg, 0.083 mmol) and Cs₂CO₃ (80.8 mg, 0.248 mmol) were placed in a vial, dissolved in dry DMF (2.0 mL) and stirred at RT for 4 days. After complete consumption of the starting material (monitored by LCMS), the solvent was evaporated under reduced pressure. The residues were dissolved in DCM and washed with H₂O and brine. The organic layer was dried over Na₂SO₄, filtered and concentrated in vacuo. The product tert-butyl 1-[2-(4-{[(tert-butoxy)carbonyl]amino}piperidin-1-yl)ethyl]-6-chloro-3-[3-(naphthalen-1-yloxy)propyl]-7-(1,3,5-trimethyl-1H-pyrazol-4-yl)-1H-indole-2-carboxylate (49.1 mg, crude) was used to next step without further purification.

LCMS (ESI+): m/z 769.9 [M+H]⁺

### Step B

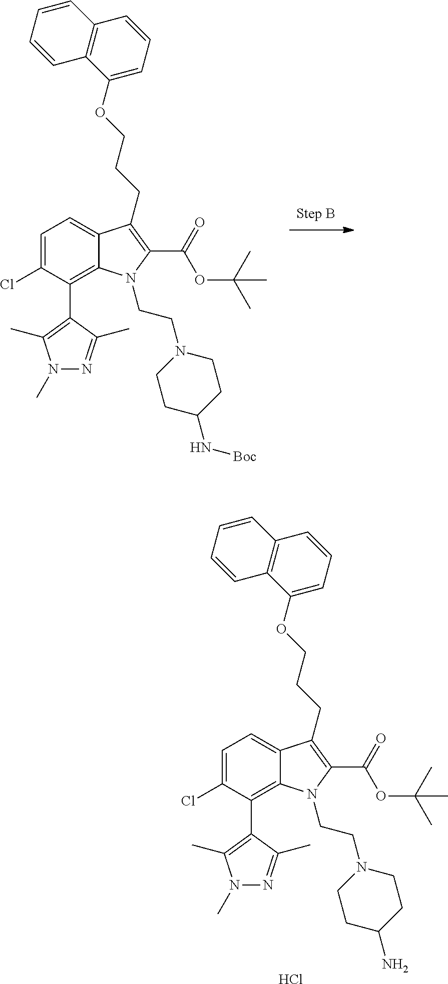 [SMILES: Cc1nn(C)c(C)c1-c1c(Cl)ccc2c(CCCOc3cccc4ccccc34)c(C(=O)OC(C)(C)C)n(CCN3CCC(NC(=O)OC(C)(C)C)CC3)c12 ; Cc1nn(C)c(C)c1-c1c(Cl)ccc2c(CCCOc3cccc4ccccc34)c(C(=O)OC(C)(C)C)n(CCN3CCC(N)CC3)c12 ; Cl]

Tert-butyl 1-[2-(4-{[(tert-butoxy)carbonyl]amino}piperidin-1-yl)ethyl]-6-chloro-3-[3-(naphthalen-1-yloxy)propyl]-7-(1,3,5-trimethyl-1H-pyrazol-4-yl)-1H-indole-2-carboxylate (49.1 mg, crude) was dissolved in THE (5.0 mL) and cooled to 0° C. To the mixture was added 4M HCl in dioxane (1.0 mL, 4.000 mmol) and a reaction was stirred at room temperature for 18 h. The crude was concentrated in vacuo, dissolved in water and freeze dried. The product tert-butyl 1-[2-(4-aminopiperidin-1-yl)ethyl]-6-chloro-3-[3-(naphthalen-1-yloxy)propyl]-7-(1,3,5-trimethyl-1H-pyrazol-4-yl)-1H-indole-2-carboxylate hydrochloride (52.3 mg, crude) was a yellowish solid which was used to next step without further purification.

LCMS (ESI+): m/z 669.9 [M+H]⁺

### Step C

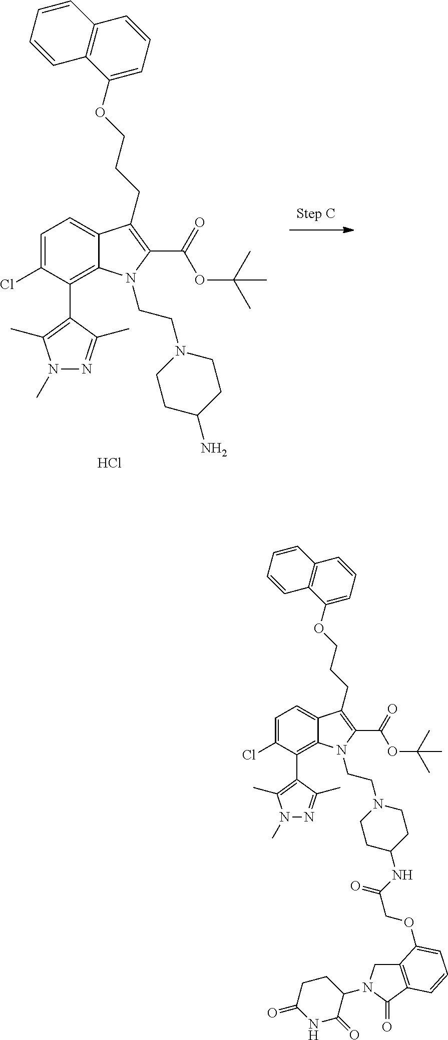 [SMILES: Cc1nn(C)c(C)c1-c1c(Cl)ccc2c(CCCOc3cccc4ccccc34)c(C(=O)OC(C)(C)C)n(CCN3CCC(N)CC3)c12 ; Cl ; Cc1nn(C)c(C)c1-c1c(Cl)ccc2c(CCCOc3cccc4ccccc34)c(C(=O)OC(C)(C)C)n(CCN3CCC(NC(=O)COc4cccc5c4CN(C4CCC(=O)NC4=O)C5=O)CC3)c12]

Tert-butyl 1-[2-(4-aminopiperidin-1-yl)ethyl]-6-chloro-3-[3-(naphthalen-1-yloxy)propyl]-7-(1,3,5-trimethyl-1H-pyrazol-4-yl)-1H-indole-2-carboxylate hydrochloride (42.2 mg, crude), 2-{[2-(2,6-dioxopiperidin-3-yl)-1-oxo-2,3-dihydro-1H-isoindol-4-yl]oxy}acetic acid (22.8 mg, 0.072 mmol) and HATU (45.4 mg, 0.119 mmol) were dissolved in dry DMF (2.0 mL) and to the mixture was added DIPEA (0.052 mL, 0.298 mmol). The reaction was stirred at RT for 2 h. The solvent was evaporated. Residues were dissolved in DCM and washed with brine. The organic layer was dried over Na₂SO₄, filtered and concentrated in vacuo. The tert-butyl 6-chloro-1-{2-[4-(2-{[2-(2,6-dioxopiperidin-3-yl)-1-oxo-2,3-dihydro-1H-isoindol-4-yl]oxy}acetamido)piperidin-1-yl]ethyl}-3-[3-(naphthalen-1-yloxy)propyl]-7-(1,3,5-trimethyl-1H-pyrazol-4-yl)-1H-indole-2-carboxylate (55 mg, crude) was a brown oil and was used without a further purification to next step.

LCMS (ESI+): m/z 969.7 [M+H]⁺

### Step D

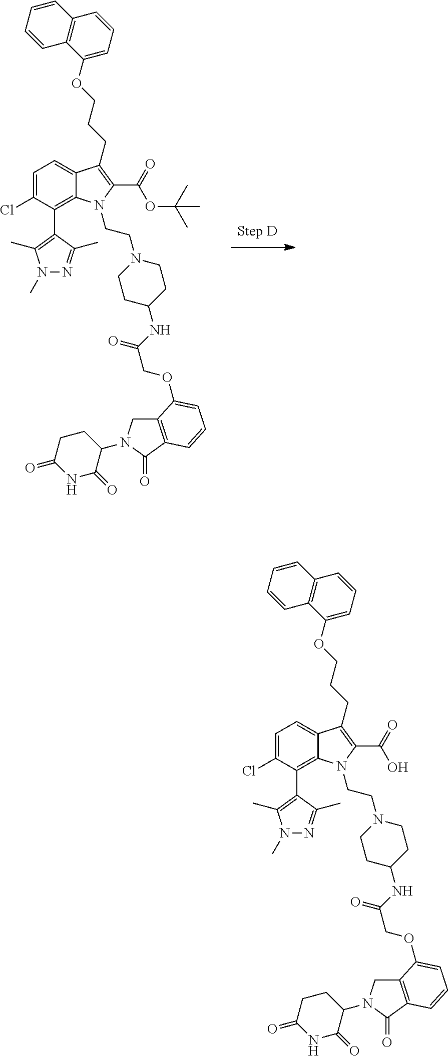 [SMILES: Cc1nn(C)c(C)c1-c1c(Cl)ccc2c(CCCOc3cccc4ccccc34)c(C(=O)OC(C)(C)C)n(CCN3CCC(NC(=O)COc4cccc5c4CN(C4CCC(=O)NC4=O)C5=O)CC3)c12 ; Cc1nn(C)c(C)c1-c1c(Cl)ccc2c(CCCOc3cccc4ccccc34)c(C(=O)O)n(CCN3CCC(NC(=O)COc4cccc5c4CN(C4CCC(=O)NC4=O)C5=O)CC3)c12]

To a solution of tert-butyl 6-chloro-1-{2-[4-(2-{[2-(2,6-dioxopiperidin-3-yl)-1-oxo-2,3-dihydro-1H-isoindol-4-yl]oxy}acetamido)piperidin-1-yl]ethyl}-3-[3-(naphthalen-1-yloxy)propyl]-7-(1,3,5-trimethyl-1H-pyrazol-4-yl)-1H-indole-2-carboxylate (55.0 mg, crude) in DCM (1.0 mL) was added TFA (0.500 mL, 6.529 mmol). The mixture was stirred for 18 h at RT. The crude was concentrated in vacuo, the residues were dissolved in DMSO and purified by preparative HPLC (H₂O:MeCN+0.1% FA) to give 6-chloro-1-{2-[4-(2-{[2-(2,6-dioxopiperidin-3-yl)-1-oxo-2,3-dihydro-1H-isoindol-4-yl]oxy}acetamido)piperidin-1-yl]ethyl}-3-[3-(naphthalen-1-yloxy)propyl]-7-(1,3,5-trimethyl-1H-pyrazol-4-yl)-1H-indole-2-carboxylic acid (8.3 mg, 0.009 mmol, 10% over 4 steps) as a white solid.

LCMS (ESI+): m/z 914.3 [M+H]⁺

¹H NMR (500 MHz, DMSO) δ 11.00 (s, 1H), 8.23 (dd, J=7.3, 2.0 Hz, 1H), 8.07 (d, J=7.8 Hz, 1H), 7.88 (dd, J=7.4, 2.0 Hz, 1H), 7.68 (d, J=8.5 Hz, 1H), 7.57-7.50 (m, 2H), 7.46 (dt, J=7.8, 3.7 Hz, 2H), 7.43-7.39 (m, 1H), 7.39-7.33 (m, 1H), 7.19 (d, J=8.5 Hz, 1H), 7.11 (d, J=8.2 Hz, 1H), 6.92 (d, J=7.5 Hz, 1H), 5.13 (dd, J=13.3, 5.1 Hz, 1H), 4.61 (d, J=3.4 Hz, 2H), 4.44 (d, J=17.5 Hz, 1H), 4.33 (d, J=17.4 Hz, 1H), 4.30-4.23 (m, 1H), 4.20 (t, J=6.3 Hz, 3H), 3.76 (s, 3H), 3.68-3.59 (m, 1H), 3.23 (dd, J=8.6, 6.5 Hz, 2H), 2.97-2.86 (m, 1H), 2.65-2.56 (m, 1H), 2.49-2.37 (m, 2H), 2.34-2.25 (m, 4H), 2.21 (p, J=6.6 Hz, 2H), 2.05-1.97 (m, 5H), 1.89 (s, 3H), 1.69-1.62 (m, 2H), 1.57-1.45 (m, 2H).

### Example 57. 6-chloro-1-{2-[5-(2-{[2-(2,6-dioxopiperidin-3-yl)-1-oxo-2,3-dihydro-1H-isoindol-4-yl]oxy}acetyl)-2,5-diazabicyclo[4.1.0]heptan-2-yl]ethyl}-3-[3-(naphthalen-1-yloxy)propyl]-7-(1,3,5-trimethyl-1H-pyrazol-4-yl)-1H-indole-2-carboxylic acid (222)

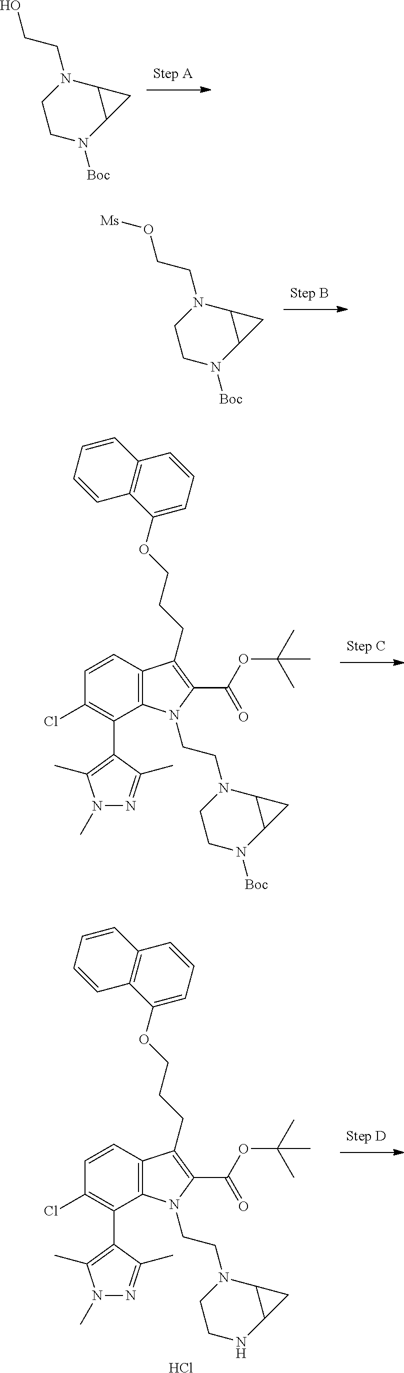

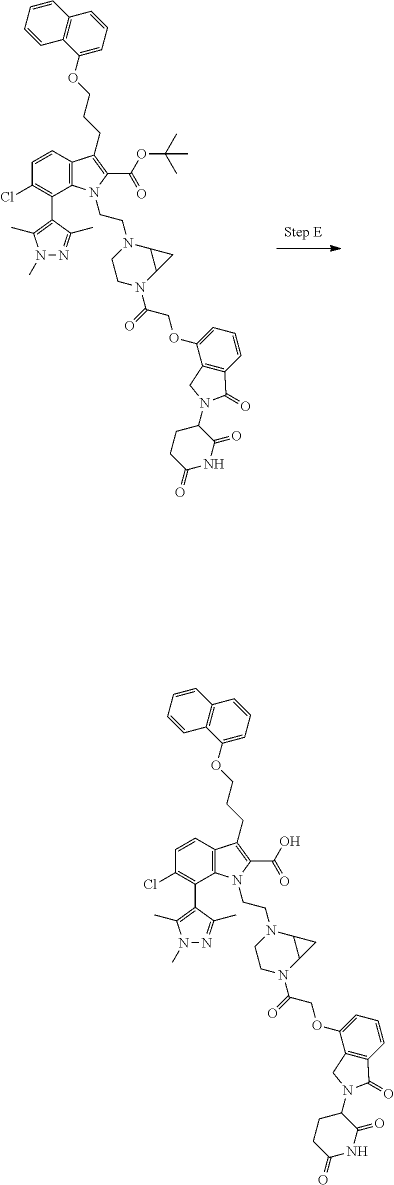 [SMILES: Cc1nn(C)c(C)c1-c1c(Cl)ccc2c(CCCOc3cccc4ccccc34)c(C(=O)OC(C)(C)C)n(CCN3CCN(C(=O)COc4cccc5c4CN(C4CCC(=O)NC4=O)C5=O)C4CC43)c12 ; Cc1nn(C)c(C)c1-c1c(Cl)ccc2c(CCCOc3cccc4ccccc34)c(C(=O)O)n(CCN3CCN(C(=O)COc4cccc5c4CN(C4CCC(=O)NC4=O)C5=O)C4CC43)c12]

### Step A

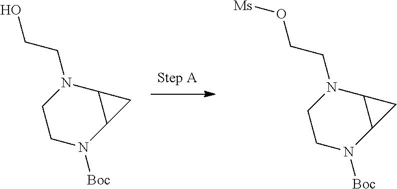 [SMILES: CC(C)(C)OC(=O)N1CCN(CCO)C2CC21 ; CC(C)(C)OC(=O)N1CCN(CCOS(C)(=O)=O)C2CC21]

Tert-butyl 5-(2-hydroxyethyl)-2,5-diazabicyclo[4.1.0]heptane-2-carboxylate (55.7 mg, 0.230 mmol) was dissolved in DCM (3.0 mL), Et₃N (0.048 mL, 0.345 mmol) and DMAP (2.8 mg, 0.023 mmol) were added and reaction mixture was cooled down to 0° C. Then, MsCl (0.021 mL, 0.276 mmol) was added dropwise and reaction mixture was let to stir at RT for 18 h. The crude was extracted with brine, dried over Na₂SO₄, filtered and concentrated in vacuo. The product tert-butyl 5-[2-(methanesulfonyloxy)ethyl]-2,5-diazabicyclo[4.1.0]heptane-2-carboxylate (78.7 mg, crude) was an orange oil and was used in the next step without further purification (TLC, 5% MeOH in DCM, R_{f}=0.7).

### Step B

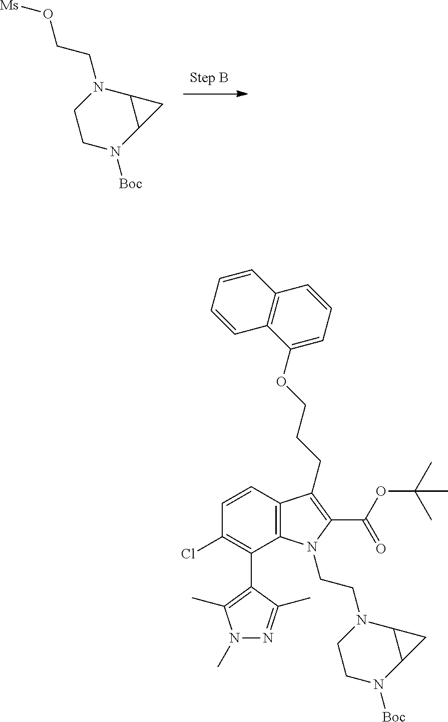 [SMILES: CC(C)(C)OC(=O)N1CCN(CCOS(C)(=O)=O)C2CC21 ; Cc1nn(C)c(C)c1-c1c(Cl)ccc2c(CCCOc3cccc4ccccc34)c(C(=O)OC(C)(C)C)n(CCN3CCN(C(=O)OC(C)(C)C)C4CC43)c12]

To a solution of tert-butyl 5-[2-(methanesulfonyloxy)ethyl]-2,5-diazabicyclo[4.1.0]heptane-2-carboxylate (88.3 mg, 0.276 mmol) and tert-butyl 6-chloro-3-[3-(naphthalen-1-yloxy)propyl]-7-(1,3,5-trimethyl-1H-pyrazol-4-yl)-1H-indole-2-carboxylate (50.0 mg, crude) in dry DMF (1.0 mL) was added Cs₂CO₃ (89.8 mg, 0.276 mmol). The mixture was stirred for 6 days at RT. To the mixture was added DCM and washed with brine. The organic layer was dried over Na₂SO₄, filtered and concentrated in vacuo. The product tert-butyl 1-(2-{5-[(tert-butoxy)carbonyl]-2,5-diazabicyclo[4.1.0]heptan-2-yl}ethyl)-6-chloro-3-[3-(naphthalen-1-yloxy)propyl]-7-(1,3,5-trimethyl-1H-pyrazol-4-yl)-1H-indole-2-carboxylate (88.7 mg, crude) was a light yellow oil, which was used in next step without further purification.

LCMS (ESI+): m/z 768.3 [M+H]⁺

### Step C

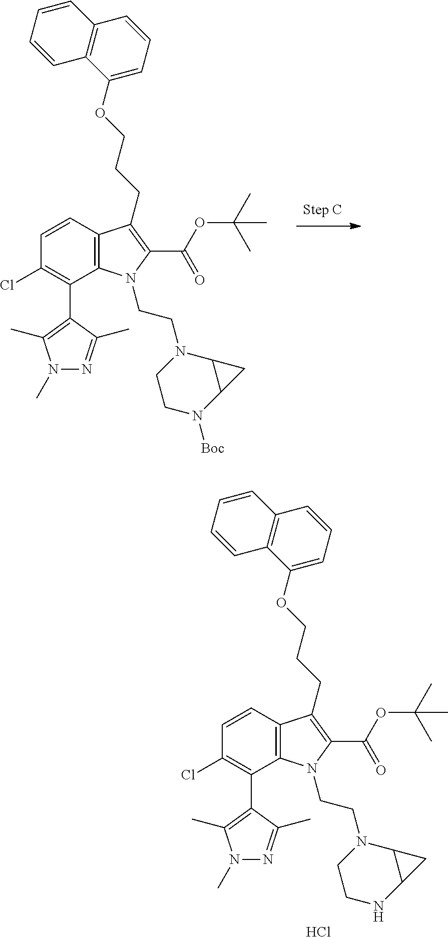 [SMILES: Cc1nn(C)c(C)c1-c1c(Cl)ccc2c(CCCOc3cccc4ccccc34)c(C(=O)OC(C)(C)C)n(CCN3CCN(C(=O)OC(C)(C)C)C4CC43)c12 ; Cc1nn(C)c(C)c1-c1c(Cl)ccc2c(CCCOc3cccc4ccccc34)c(C(=O)OC(C)(C)C)n(CCN3CCNC4CC43)c12 ; Cl]

tert-butyl 1-(2-{5-[(tert-butoxy)carbonyl]-2,5-diazabicyclo[4.1.0]heptan-2-yl}ethyl)-6-chloro-3-[3-(naphthalen-1-yloxy)propyl]-7-(1,3,5-trimethyl-1H-pyrazol-4-yl)-1H-indole-2-carboxylate (102.0 mg, crude) was dissolved in THE (2.0 mL) and cooled to 0° C. To the mixture was added 4M HCl in dioxane (0.184 mL, 5.310 mmol) and a reaction was stirred at room temperature for 18 h. The crude was concentrated in vacuo. The product tert-butyl 6-chloro-1-(2-{2,5-diazabicyclo[4.1.0]heptan-2-yl}ethyl)-3-[3-(naphthalen-1-yloxy)propyl]-7-(1,3,5-trimethyl-1H-pyrazol-4-yl)-1H-indole-2-carboxylate hydrochloride (90.0 mg, crude) was a yellowish solid which was used in next step without further purification.

LCMS (ESI+): m/z 668.3 [M+H]⁺

### Step D

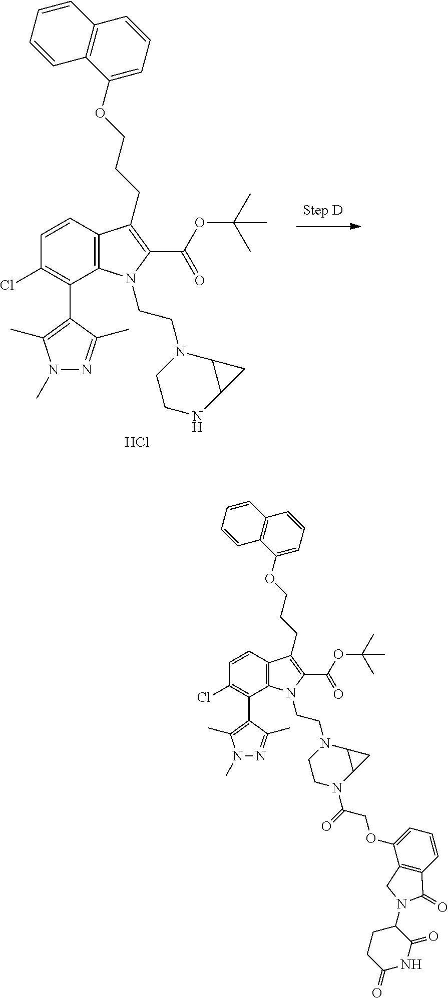 [SMILES: Cc1nn(C)c(C)c1-c1c(Cl)ccc2c(CCCOc3cccc4ccccc34)c(C(=O)OC(C)(C)C)n(CCN3CCNC4CC43)c12 ; Cl ; Cc1nn(C)c(C)c1-c1c(Cl)ccc2c(CCCOc3cccc4ccccc34)c(C(=O)OC(C)(C)C)n(CCN3CCN(C(=O)COc4cccc5c4CN(C4CCC(=O)NC4=O)C5=O)C4CC43)c12]

Tert-butyl 6-chloro-1-(2-{2,5-diazabicyclo[4.1.0]heptan-2-yl}ethyl)-3-[3-(naphthalen-1-yloxy)propyl]-7-(1,3,5-trimethyl-1H-pyrazol-4-yl)-1H-indole-2-carboxylate hydrochloride (35.0 mg, crude), 2-{[2-(2,6-dioxopiperidin-3-yl)-1-oxo-2,3-dihydro-1H-isoindol-4-yl]oxy}acetic acid (19.0 mg, 0.060 mmol) and HATU (37.8 mg, 0.099 mmol) was dissolved in dry DMF (2.0 mL) and to the mixture was added DIPEA (0.043 mL, 0.248 mmol). The reaction was stirred at RT for 2 h. The solvent was evaporated. Residues were dissolved in DCM and washed with brine. The organic layer was dried over Na₂SO₄, filtered and concentrated in vacuo. The product tert-butyl 6-chloro-1-{2-[5-(2-{[2-(2,6-dioxopiperidin-3-yl)-1-oxo-2,3-dihydro-1H-isoindol-4-yl]oxy}acetyl)-2,5-diazabicyclo[4.1.0]heptan-2-yl]ethyl}-3-[3-(naphthalen-1-yloxy)propyl]-7-(1,3,5-trimethyl-1H-pyrazol-4-yl)-1H-indole-2-carboxylate (50.0 mg, crude) was used in next step without further purification.

LCMS (ESI+): m/z 967.9 [M+H]⁺

### Step E

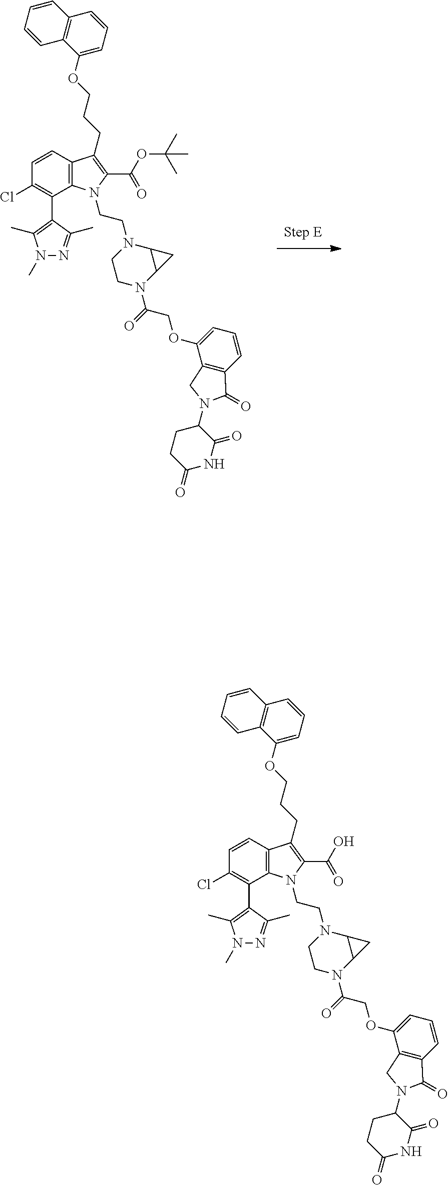 [SMILES: Cc1nn(C)c(C)c1-c1c(Cl)ccc2c(CCCOc3cccc4ccccc34)c(C(=O)OC(C)(C)C)n(CCN3CCN(C(=O)COc4cccc5c4CN(C4CCC(=O)NC4=O)C5=O)C4CC43)c12 ; Cc1nn(C)c(C)c1-c1c(Cl)ccc2c(CCCOc3cccc4ccccc34)c(C(=O)O)n(CCN3CCN(C(=O)COc4cccc5c4CN(C4CCC(=O)NC4=O)C5=O)C4CC43)c12]

To a solution of tert-butyl 6-chloro-1-{2-[5-(2-{[2-(2,6-dioxopiperidin-3-yl)-1-oxo-2,3-dihydro-1H-isoindol-4-yl]oxy}acetyl)-2,5-diazabicyclo[4.1.0]heptan-2-yl]ethyl}-3-[3-(naphthalen-1-yloxy)propyl]-7-(1,3,5-trimethyl-1H-pyrazol-4-yl)-1H-indole-2-carboxylate (50.0 mg, crude) in DCM (1.0 mL) was added TFA (0.500 mL, 6.529 mmol). The mixture was stirred for 18 h at RT. The crude was concentrated in vacuo and purified by preparative HPLC (H₂O:MeCN+0.1% FA) to give 6-chloro-1-{2-[5-(2-{[2-(2,6-dioxopiperidin-3-yl)-1-oxo-2,3-dihydro-1H-isoindo-4-yl]oxy}acetyl)-2,5-diazabicyclo[4.1.0]heptan-2-yl]ethyl}-3-[3-(naphthalen-1-yloxy)propyl]-7-(1,3,5-trimethyl-1H-pyrazol-4-yl)-1H-indole-2-carboxylic acid (3.9 mg, 0.004 mmol, 8% over 5 steps) as a white solid.

LCMS (ESI+): m/z 912.3 [M+H]⁺

¹H NMR (500 MHz, DMSO) δ 13.24 (s, 1H), 10.97 (s, 1H), 8.20 (d, J=7.2 Hz, 1H), 7.86 (dd, J=8.1, 1.5 Hz, 1H), 7.71 (d, J=9.0 Hz, 1H), 7.54-7.47 (m, 2H), 7.48-7.42 (m, 2H), 7.40-7.36 (m, 1H), 7.31 (d, J=7.4 Hz, 1H), 7.20 (d, J=8.4 Hz, 1H), 7.10 (d, J=8.1 Hz, 1H), 6.90 (d, J=7.5 Hz, 1H), 5.19-4.93 (m, 3H), 4.45-4.15 (m, 7H), 3.75-3.67 (m, 4H), 2.96-2.84 (m, 2H), 2.77-2.65 (m, 1H), 2.62-2.27 (m, 3H), 2.22 (p, J=7.0 Hz, 2H), 2.14-2.07 (m, 1H), 2.05-1.96 (m, 6H), 1.89 (d, J=8.4 Hz, 3H), 0.62-0.50 (m, 2H).

2H in aliphatic area overlaps with water

### Example 58. 6-chloro-1-{2-[1-(2-{[2-(2,6-dioxopiperidin-3-yl)-1-oxo-2,3-dihydro-1H-isoindol-4-yl]oxy}acetyl)piperidin-4-yl]ethyl}-3-[3-(naphthalen-1-yloxy)propyl]-7-(1,3,5-trimethyl-1H-pyrazol-4-yl)-1H-indole-2-carboxylic acid (223)

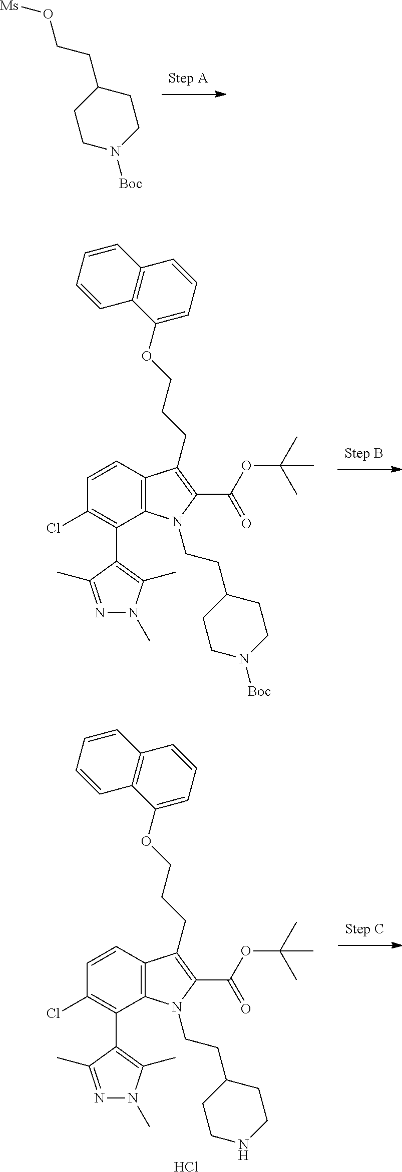

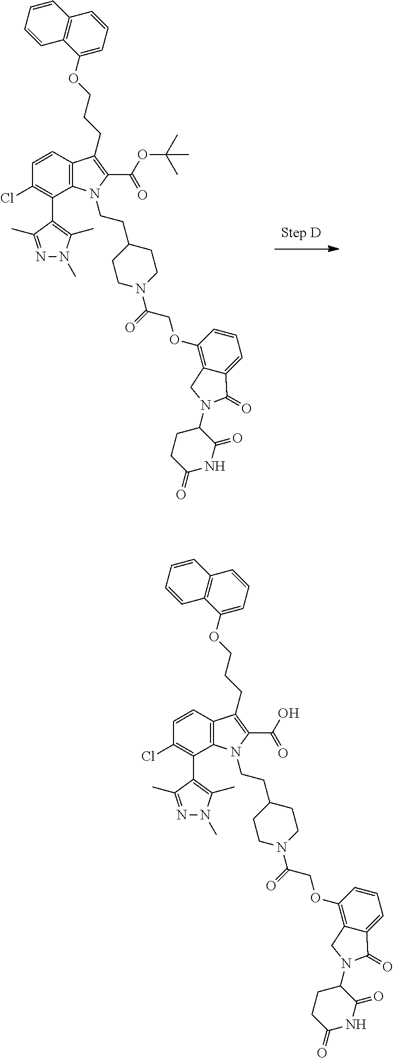 [SMILES: Cc1nn(C)c(C)c1-c1c(Cl)ccc2c(CCCOc3cccc4ccccc34)c(C(=O)OC(C)(C)C)n(CCC3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4ccccc34)c(C(=O)O)n(CCC3CCN(C(=O)COc4cccc5c4CN(C4CCC(=O)NC4=O)C5=O)CC3)c12]

### Step A

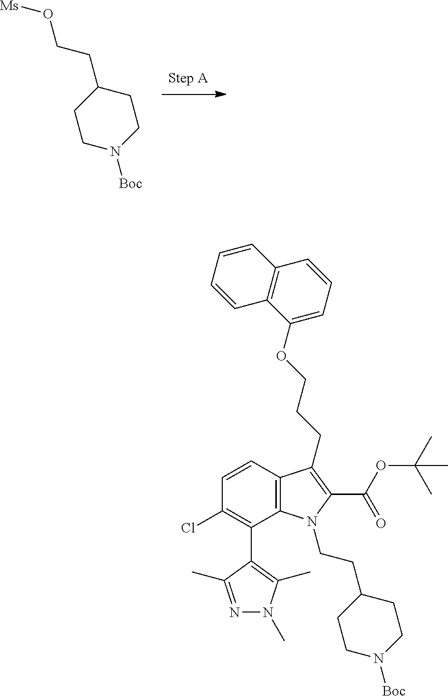 [SMILES: CC(C)(C)OC(=O)N1CCC(CCOS(C)(=O)=O)CC1 ; Cc1nn(C)c(C)c1-c1c(Cl)ccc2c(CCCOc3cccc4ccccc34)c(C(=O)OC(C)(C)C)n(CCC3CCN(C(=O)OC(C)(C)C)CC3)c12]

N-Boc-4-(2-bromo-ethyl)-piperidine (25.8 mg, 0.088 mmol), tert-butyl 6-chloro-3-[3-(naphthalen-1-yloxy)propyl]-7-(1,3,5-trimethyl-1H-pyrazol-4-yl)-1H-indole-2-carboxylate (40.0 mg, 0.074 mmol) and Cs₂CO₃ (71.9 mg, 0.221 mmol) were placed in a vial, dissolved in dry DMF (2.0 mL) and stirred at RT for 4 days. After complete consumption of the starting material (monitored by LCMS), the solvent was evaporated under reduced pressure, the residues were dissolved in DCM and washed with H₂O and brine. The organic layer was dried over Na₂SO₄, filtered and concentrated in vacuo. The product tert-butyl 1-(2-{1-[(tert-butoxy)carbonyl]piperidin-4-yl}ethyl)-6-chloro-3-[3-(naphthalen-1-yloxy)propyl]-7-(1,3,5-trimethyl-1H-pyrazol-4-yl)-1H-indole-2-carboxylate (49.1 mg, crude) was used in the next step without further purification.

LCMS (ESI+): m/z 754.9 [M+H]⁺

### Step B

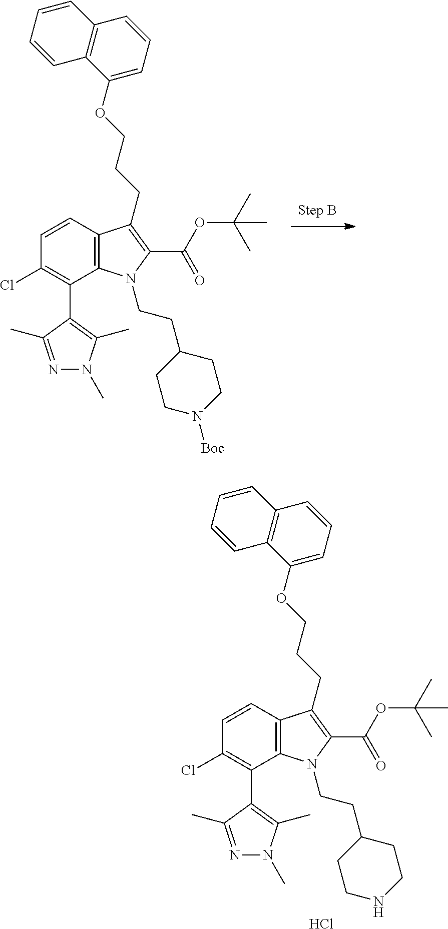 [SMILES: Cc1nn(C)c(C)c1-c1c(Cl)ccc2c(CCCOc3cccc4ccccc34)c(C(=O)OC(C)(C)C)n(CCC3CCN(C(=O)OC(C)(C)C)CC3)c12 ; Cc1nn(C)c(C)c1-c1c(Cl)ccc2c(CCCOc3cccc4ccccc34)c(C(=O)OC(C)(C)C)n(CCC3CCNCC3)c12 ; Cl]

To a solution of tert-butyl 1-(2-{1-[(tert-butoxy)carbonyl]piperidin-4-yl}ethyl)-6-chloro-3-[3-(naphthalen-1-yloxy)propyl]-7-(1,3,5-trimethyl-1H-pyrazol-4-yl)-1H-indole-2-carboxylate (49.1 mg, crude) in THE (5.0 mL) at 0° C. was added 4M HCl in dioxane (2.0 mL, 8.000 mmol). The mixture was stirred for next 36 h at RT. After complete consumption of the substrate (monitored by LCMS), the crude was concentrated in vacuo and tert-butyl 6-chloro-3-[3-(naphthalen-1-yloxy)propyl]-1-[2-(piperidin-4-yl)ethyl]-7-(1,3,5-trimethyl-1H-pyrazol-4-yl)-1H-indole-2-carboxylate hydrochloride (52.3 mg, crude) was used in the next step without further purification.

LCMS (ESI+): m/z 655.1 [M+H]⁺

### Step C

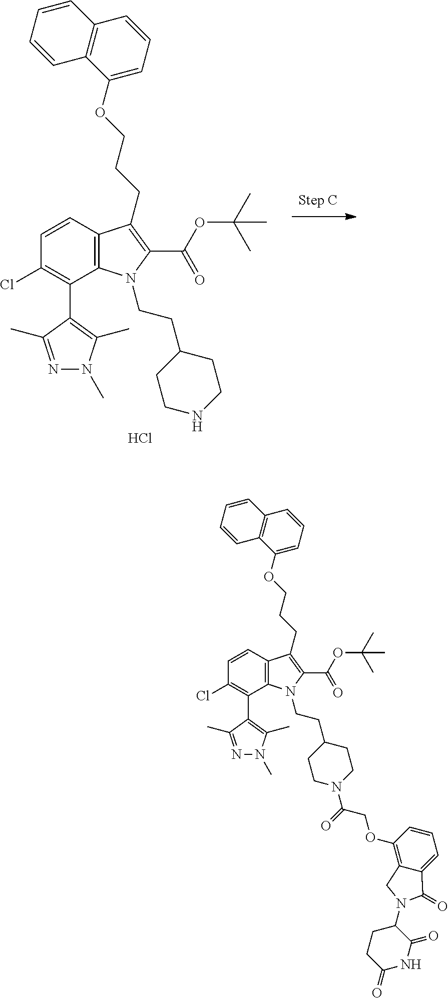 [SMILES: Cc1nn(C)c(C)c1-c1c(Cl)ccc2c(CCCOc3cccc4ccccc34)c(C(=O)OC(C)(C)C)n(CCC3CCNCC3)c12 ; Cl ; Cc1nn(C)c(C)c1-c1c(Cl)ccc2c(CCCOc3cccc4ccccc34)c(C(=O)OC(C)(C)C)n(CCC3CCN(C(=O)COc4cccc5c4CN(C4CCC(=O)NC4=O)C5=O)CC3)c12]

2-{4-[(2,6-Dioxopiperidin-3-yl)carbamoyl]-2-methyl-1H-1,3-benzodiazol-1-yl}acetic acid (14.6 mg, 0.046 mmol), tert-butyl 6-chloro-3-[3-(naphthalen-1-yloxy)propyl]-1-[2-(piperidin-4-yl)ethyl]-7-(1,3,5-trimethyl-1H-pyrazol-4-yl)-1H-indole-2-carboxylate hydrochloride (25.0 mg, crude) and HATU (29.0 mg, 0.076 mmol) were dissolved in dry DMF (2.0 mL) and to the mixture was added DIPEA (0.033 mL, 0.191 mmol). The reaction was stirred at RT for 30 min. The solvent was evaporated. Residues were dissolved in DCM and washed with brine. The organic layer was dried over Na₂SO₄, filtered and concentrated in vacuo. The product tert-butyl 6-chloro-1-{2-[1-(2-{[2-(2,6-dioxopiperidin-3-yl)-1-oxo-2,3-dihydro-1H-isoindol-4-yl]oxy}acetyl)piperidin-4-yl]ethyl}-3-[3-(naphthalen-1-yloxy)propyl]-7-(1,3,5-trimethyl-1H-pyrazol-4-yl)-1H-indole-2-carboxylate (44 mg, crude) was used without further purification in the next step.

LCMS (ESI+): m/z 954.7 [M+H]⁺

### Step D

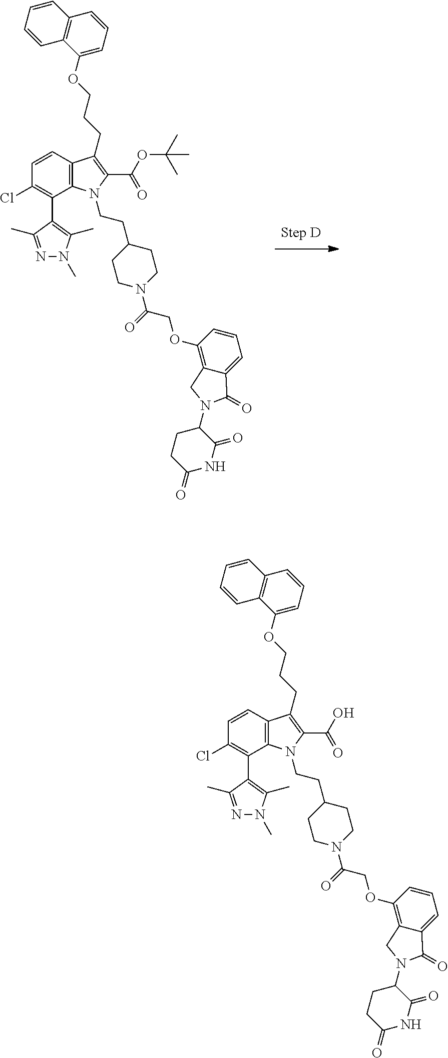 [SMILES: Cc1nn(C)c(C)c1-c1c(Cl)ccc2c(CCCOc3cccc4ccccc34)c(C(=O)OC(C)(C)C)n(CCC3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4ccccc34)c(C(=O)O)n(CCC3CCN(C(=O)COc4cccc5c4CN(C4CCC(=O)NC4=O)C5=O)CC3)c12]

To a solution of tert-butyl 6-chloro-1-{2-[1-(2-{[2-(2,6-dioxopiperidin-3-yl)-1-oxo-2,3-dihydro-1H-isoindol-4-yl]oxy}acetyl)piperidin-4-yl]ethyl}-3-[3-(naphthalen-1-yloxy)propyl]-7-(1,3,5-trimethyl-1H-pyrazol-4-yl)-1H-indole-2-carboxylate (44.0 mg, crude) in DCM (1.0 mL) was added TFA (0.500 mL, 6.529 mmol). The mixture was stirred for 18 h at RT. The crude was concentrated in vacuo. The residues were dissolved in DMSO and purified by preparative HPLC (H₂O:MeCN+0.1% FA) to give 6-chloro-1-{2-[1-(2-{[2-(2,6-dioxopiperidin-3-yl)-1-oxo-2,3-dihydro-1H-isoindol-4-yl]oxy}acetyl)piperidin-4-yl]ethyl}-3-[3-(naphthalen-1-yloxy)propyl]-7-(1,3,5-trimethyl-1H-pyrazol-4-yl)-1H-indole-2-carboxylic acid (7.0 mg, 0.008 mmol, 11% over 4 steps) as a white solid.

LCMS (ESI+): m/z 899.4 [M+H]⁺

¹H NMR (500 MHz, DMSO) δ 12.52 (s, 1H), 10.63 (s, 1H), 8.20 (dd, J=7.9, 1.8 Hz, 1H), 7.84 (dd, J=7.6, 1.8 Hz, 1H), 7.70 (d, J=8.5 Hz, 1H), 7.53-7.46 (m, 2H), 7.46-7.42 (m, 2H), 7.37 (t, J=7.9 Hz, 1H), 7.33 (d, J=7.5 Hz, 1H), 7.18 (dd, J=11.6, 8.3 Hz, 2H), 6.90 (d, J=7.7 Hz, 1H), 5.05 (dd, J=13.0, 5.2 Hz, 1H), 4.90 (s, 2H), 4.45-4.38 (m, 1H), 4.38-4.31 (m, 1H), 4.24 (t, J=6.3 Hz, 2H), 4.22-4.17 (m, 1H), 4.14-4.07 (m, 1H), 3.75 (d, J=3.5 Hz, 3H), 3.32-3.26 (m, 2H), 2.93-2.82 (m, 4H), 2.67-2.60 (m, 1H), 2.48-2.42 (m, 2H), 2.27-2.20 (m, 2H), 2.09-2.01 (m, 2H), 2.00 (s, 3H), 1.88 (s, 3H), 1.46-1.36 (m, 2H), 1.25-1.19 (m, 1H), 1.17-1.07 (m, 2H).

### Example 59. 1-(2-(2-carboxy-6-chloro-3-(3-(naphthalen-1-yloxy)propyl)-7-(1,3,5-trimethyl-1H-pyrazol-4-yl)-1H-indol-1-yl)ethyl)-4-(2-((2-(2,6-dioxopiperidin-3-yl)-1-oxoisoindolin-4-yl)oxy)acetyl)piperazine 1-oxide (224)

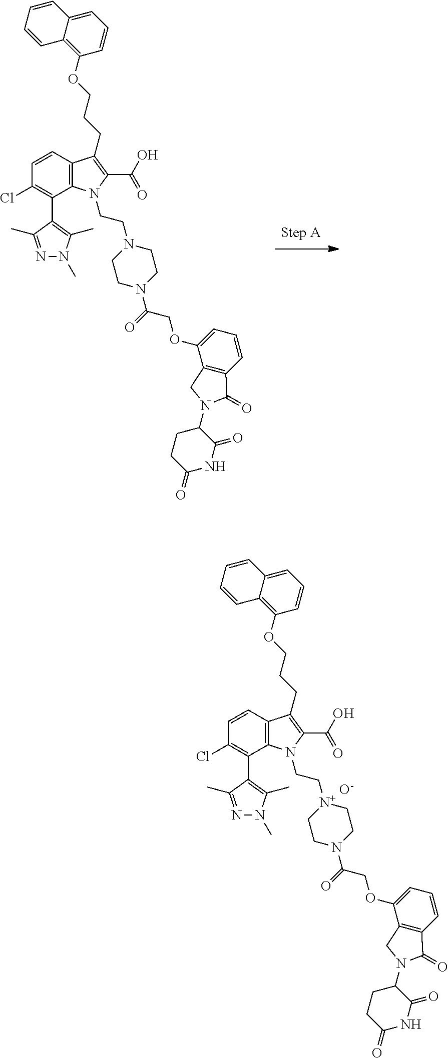

### Step A

6-chloro-1-(2-(4-(2-((2-(2,6-dioxopiperidin-3-yl)-1-oxoisoindolin-4-yl)oxy)acetyl)piperazin-1-yl)ethyl)-3-(3-(naphthalen-1-yloxy)propyl)-7-(1,3,5-trimethyl-1H-pyrazol-4-yl)-1H-indole-2-carboxylic acid (5.0 mg, 0.006 mmol) was dissolved in ethyl acetate (222 μL) and the mixture was cooled down to 0° C. m-CPBA (1.5 mg, 0.006 mmol) was added and after 5 min mixture was filtrated through SiO₂ pad. Reaction product was washed from silica with MeOH and crude product was purified using preparative HPLC (H₂O:MeCN+0.1% FA). 1-(2-(2-carboxy-6-chloro-3-(3-(naphthalen-1-yloxy)propyl)-7-(1,3,5-trimethyl-1H-pyrazol-4-yl)-1H-indol-1-yl)ethyl)-4-(2-((2-(2,6-dioxopiperidin-3-yl)-1-oxoisoindolin-4-yl)oxy)acetyl)piperazine 1-oxide (1.3 mg, 0.001 mmol, 24.6%) was isolated as white solid.

LCMS (ESI+): m/z 916.3 [M+H]⁺

¹H NMR (500 MHz, DMSO) δ 10.98 (s, 1H), 8.25-8.15 (m, 2H), 7.90-7.81 (m, 1H), 7.55-7.47 (m, 2H), 7.47-7.40 (m, 2H), 7.40-7.28 (m, 2H), 7.21-7.06 (m, 2H), 6.95-6.85 (m, 1H), 5.11 (dd, J=13.2, 5.1 Hz, 1H), 4.95 (s, 2H), 4.49-4.07 (m, 4H), 3.77-3.72 (m, 3H), 3.40-3.36 (m, 3H), 2.97-2.85 (m, 1H), 2.61-2.52 (m, 2H), 2.46-2.45 (m, 2H), 2.24-2.15 (m, 2H), 2.15-2.08 (m, 2H), 2.07-1.92 (m, 9H), 1.87 (d, J=3.0 Hz, 3H).

### Example 60. 1-(2-(4-(2-((2-(2,6-dioxopiperidin-3-yl)-1-oxoisoindolin-4-yl)oxy)acetyl)piperazin-1-yl)ethyl)-6-fluoro-3-(3-((6-fluoronaphthalen-1-yl)oxy)propyl)-7-(2-methyl-5,6-dihydro-4H-pyrrolo[1,2-b]pyrazol-3-yl)-1H-indole-2-carboxylic acid (225)

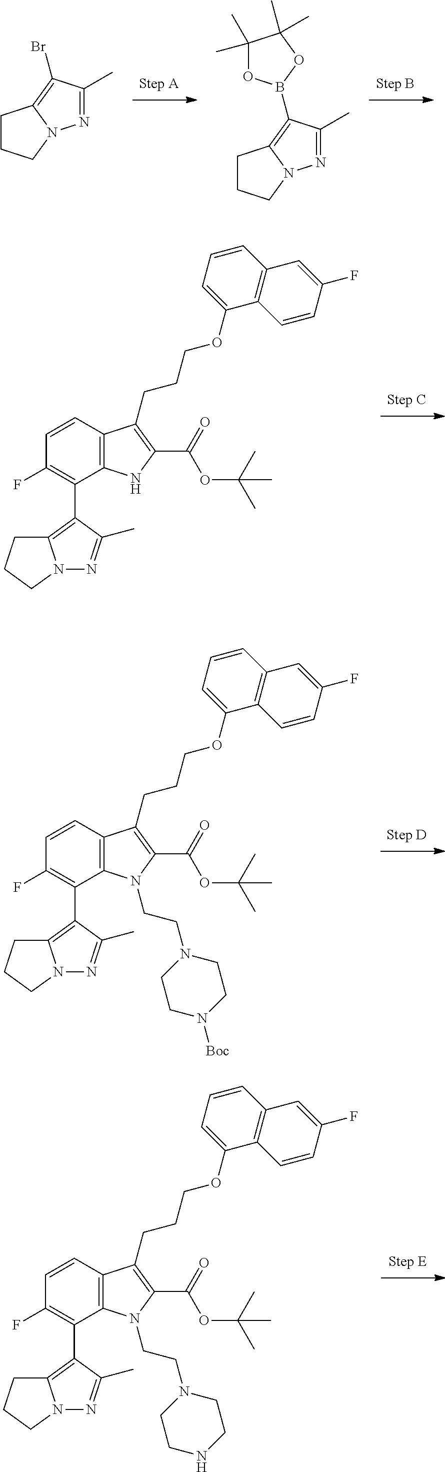

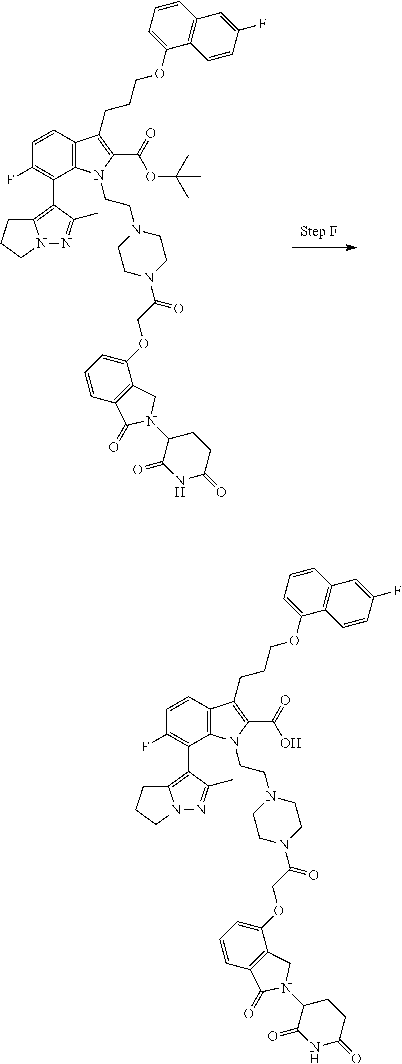 [SMILES: Cc1nn2c(c1-c1c(F)ccc3c(CCCOc4cccc5cc(F)ccc45)c(C(=O)OC(C)(C)C)n(CCN4CCN(C(=O)COc5cccc6c5CN(C5CCC(=O)NC5=O)C6=O)CC4)c13)CCC2 ; Cc1nn2c(c1-c1c(F)ccc3c(CCCOc4cccc5cc(F)ccc45)c(C(=O)O)n(CCN4CCN(C(=O)COc5cccc6c5CN(C5CCC(=O)NC5=O)C6=O)CC4)c13)CCC2]

### Step A

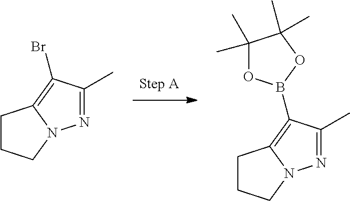

To a well stirred solution of 3-bromo-2-methyl-5,6-dihydro-4H-pyrrolo[1,2-b]pyrazole (1.2 g, 6.03 mmol) and 2-isopropoxy-4,4,5,5-tetramethyl-1,3,2-dioxaborolane (2.4 mL, 18.09 mmol) in THE (25 mL), was added n-butyllithium (1.8 M, 8 mL, 14.472 mmol) at −78° C. under argon. The resulting mixture was then allowed to stir at −78° C. for 2 h. The mixture was slowly warmed to RT and was stirred for another 30 minutes. After complete consumption of the starting material (monitored by TLC and LCMS) the excess n-butyllithium was quenched by slow addition of saturated ammonium chloride solution. It was then diluted with EtOAc, washed successively with water and brine. The organic layer was dried over Na₂SO₄ and evaporated under reduced pressure to get the crude compound, which was then purified by column chromatography (SiO₂, EtOAc:hexane, 30-40%) to get 2-methyl-3-(4,4,5,5-tetramethyl-1,3,2-dioxaborolan-2-yl)-5,6-dihydro-4H-pyrrolo[1,2-b]pyrazole (650 mg, 2.62 mmol, 44%) as white sticky solid.

LCMS (ESI+): m/z 247.8 [M+H]⁺

### Step B

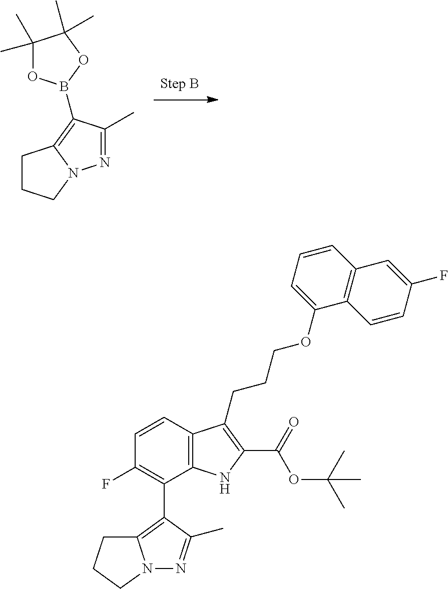

To a stirred solution of tert-butyl 7-bromo-6-fluoro-3-(3-((6-fluoronaphthalen-1-yl)oxy)propyl)-1H-indole-2-carboxylate (700 mg, 1.357 mmol) in dioxane (12 mL) and water (3 mL) were added 2-methyl-3-(4,4,5,5-tetramethyl-1,3,2-dioxaborolan-2-yl)-5,6-dihydro-4H-pyrrolo[1,2-b]pyrazole (672 mg, 2.713 mmol) followed by potassium phosphate (862 mg, 4.07 mmol). The mixture was deoxygenated with argon and to it was added [1,1′-Bis(di-tert-butylphosphino)ferrocene]dichloro palladium(II) (176 mg, 0.0.271 mmol) under argon atmosphere. Then the reaction mixture was heated under reflux for 16 h. After complete consumption of the starting material (monitored by TLC and LCMS) the reaction mixture was filtered through celite pad and the filtrate was evaporated under reduced pressure get the crude material. It was then diluted with EtOAc, washed successively with water and brine, the organic layer was dried over Na₂SO₄ and evaporated under reduced pressure to get the crude compound, which was then purified by column chromatography (SiO2, 30% EtOAc in DCM) to get tert-butyl 6-fluoro-3-(3-((6-fluoronaphthalen-1-yl)oxy)propyl)-7-(2-methyl-5,6-dihydro-4H-pyrrolo[1,2-b]pyrazol-3-yl)-1H-indole-2-carboxylate (650 mg, 1.166 mmol, 86%) as brown solid.

LCMS (ESI+): m/z 558.0 [M+H]⁺

### Step C

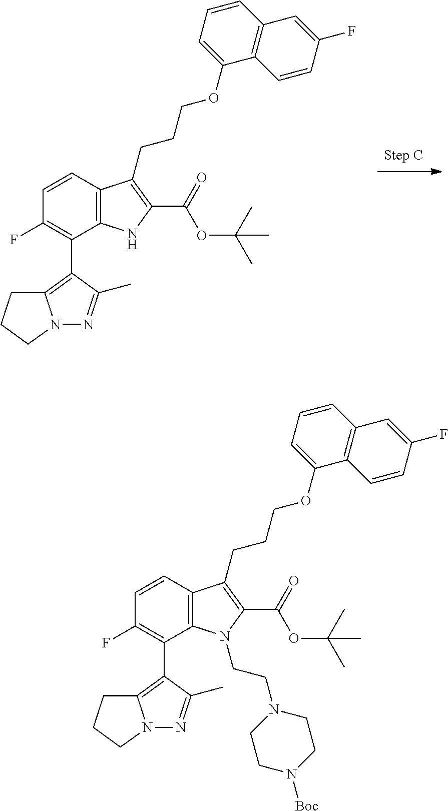 [SMILES: Cc1nn2c(c1-c1c(F)ccc3c(CCCOc4cccc5cc(F)ccc45)c(C(=O)OC(C)(C)C)[nH]c13)CCC2 ; Cc1nn2c(c1-c1c(F)ccc3c(CCCOc4cccc5cc(F)ccc45)c(C(=O)OC(C)(C)C)n(CCN4CCN(C(=O)OC(C)(C)C)CC4)c13)CCC2]

To a well stirred solution of tert-butyl 6-fluoro-3-(3-((6-fluoronaphthalen-1-yl)oxy)propyl)-7-(2-methyl-5,6-dihydro-4H-pyrrolo[1,2-b]pyrazol-3-yl)-1H-indole-2-carboxylate (650 mg, 1.167 mmol) in DMF (8 mL) was added tert-butyl 4-(2-chloroethyl)piperazine-1-carboxylate (377 mg, 1.517 mmol), followed by Cs₂CO₃ (568 mg, 1.75 mmol) in DMF and the mixture was allowed to stir at 90° C. for 16 h under nitrogen. After complete consumption of the starting material (monitored by TLC and LCMS) the reaction mixture was diluted with EtOAc and washed successively with water and brine. The organic layer was dried over Na₂SO₄ and evaporated under reduced pressure to get the crude compound, which was then purified by column chromatography (SiO₂, 40% EtOAc in DCM) to get tert-butyl 1-(2-(4-(tert-butoxycarbonyl)piperazin-1-yl)ethyl)-6-fluoro-3-(3-((6-fluoronaphthalen-1-yl)oxy)propyl)-7-(2-methyl-5,6-dihydro-4H-pyrrolo[1,2-b]pyrazol-3-yl)-1H-indole-2-carboxylate (635 mg, 0.825 mmol, 71%) as white solid.

LCMS (ESI+): m/z 770.4 [M+H]⁺

### Step D

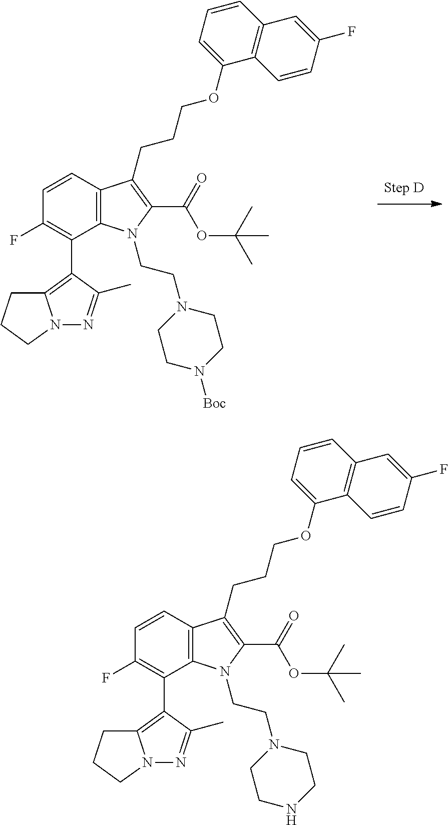 [SMILES: Cc1nn2c(c1-c1c(F)ccc3c(CCCOc4cccc5cc(F)ccc45)c(C(=O)OC(C)(C)C)n(CCN4CCN(C(=O)OC(C)(C)C)CC4)c13)CCC2 ; Cc1nn2c(c1-c1c(F)ccc3c(CCCOc4cccc5cc(F)ccc45)c(C(=O)OC(C)(C)C)n(CCN4CCNCC4)c13)CCC2]

tert-butyl 1-(2-(4-(tert-butoxycarbonyl)piperazin-1-yl)ethyl)-6-fluoro-3-(3-((6-fluoronaphthalen-1-yl)oxy)propyl)-7-(2-methyl-5,6-dihydro-4H-pyrrolo[1,2-b]pyrazol-3-yl)-1H-indole-2-carboxylate (630 mg, 0.819 mmol) was dissolved in dioxane (20 mL). To it was added 4M HCl in dioxane (10 mL) drop wise at 0° C. under nitrogen and the mixture was allowed to stir for 2 h at same temperature. Whereupon LCMS indicated the reaction was complete, the reaction mixture was quenched by dropwise addition of cold 1M NaOH solution at 0° C. to maintain the pH˜7 to 8. The aqueous layer was extracted 2-3 times with DCM. The combined organics were dried over Na₂SO₄ and concentrated in vacuum to get the crude compound which was then purified by column chromatography (amine SiO₂, 70% EtOAc in DCM) to get tert-butyl 6-fluoro-3-(3-((6-fluoronaphthalen-1-yl)oxy)propyl)-7-(2-methyl-5,6-dihydro-4H-pyrrolo[1,2-b]pyrazol-3-yl)-1-(2-(piperazin-1-yl)ethyl)-1H-indole-2-carboxylate (343 mg, 0.512 mmol, 62%) as white solid.

LCMS (ESI+): m/z 670.5 [M+H]⁺

### Step E

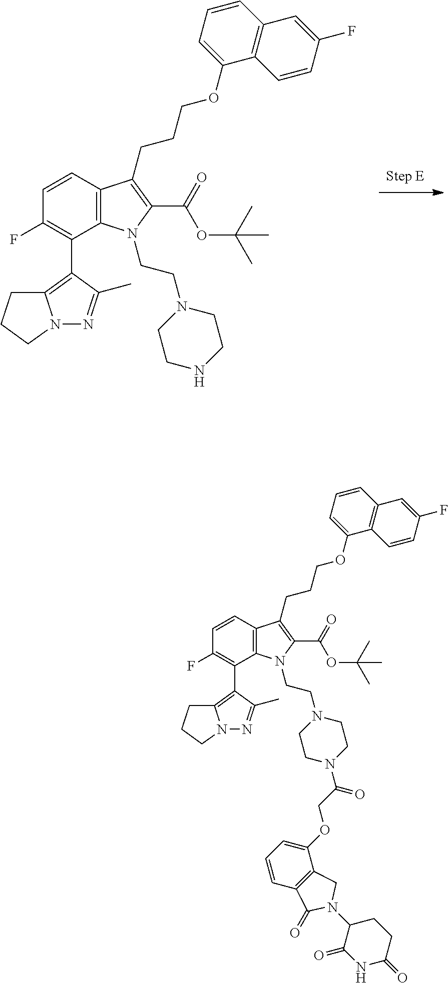 [SMILES: Cc1nn2c(c1-c1c(F)ccc3c(CCCOc4cccc5cc(F)ccc45)c(C(=O)OC(C)(C)C)n(CCN4CCNCC4)c13)CCC2 ; Cc1nn2c(c1-c1c(F)ccc3c(CCCOc4cccc5cc(F)ccc45)c(C(=O)OC(C)(C)C)n(CCN4CCN(C(=O)COc5cccc6c5CN(C5CCC(=O)NC5=O)C6=O)CC4)c13)CCC2]

2-{[2-(2,6-dioxopiperidin-3-yl)-1-oxo-2,3-dihydro-1H-isoindol-4-yl]oxy}acetic acid (26.1 mg, 0.082 mmol) was dissolved in dry DMF under argon atmosphere and DIPEA (0.039 mL, 0.224 mmol) was added along with, tert-butyl 6-fluoro-3-(3-((6-fluoronaphthalen-1-yl)oxy)propyl)-7-(2-methyl-5,6-dihydro-4H-pyrrolo[1,2-b]pyrazol-3-yl)-1-(2-(piperazin-1-yl)ethyl)-1H-indole-2-carboxylate (50.0 mg, 0.075 mmol) as a solution in DMF (1 mL). Reaction mixture was cooled down to 0° C. and HATU (31.2 mg, 0.082 mmol) in DMF (1 mL) was added dropwise. The reaction was allowed to slowly warm to room temperature and mixed for 15 min (monitored with LCMS) under argon atmosphere. After complete consumption of the starting material the reaction was quenched with aqueous saturated solution of NaHCO₃, diluted with DCM and brine. Organic layer was again washed with aqueous NaHCO₃, brine and water, collected and dried over anhydrous MgSO₄. The solution was filtrated, concentrated and dried under reduced pressure to give crude tert-butyl 1-(2-(4-(2-((2-(2,6-dioxopiperidin-3-yl)-1-oxoisoindolin-4-yl)oxy)acetyl)piperazin-1-yl)ethyl)-6-fluoro-3-(3-((6-fluoronaphthalen-1-yl)oxy)propyl)-7-(2-methyl-5,6-dihydro-4H-pyrrolo[1,2-b]pyrazol-3-yl)-1H-indole-2-carboxylate (72.5 mg) as yellow oil which was used in the next step without further purification.

LCMS (ESI+): m/z 969.7 [M+H]⁺

### Step F

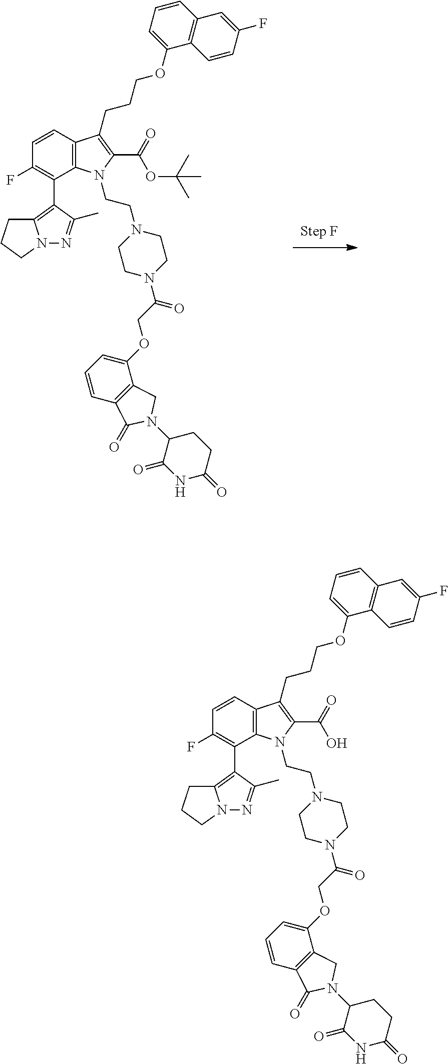 [SMILES: Cc1nn2c(c1-c1c(F)ccc3c(CCCOc4cccc5cc(F)ccc45)c(C(=O)OC(C)(C)C)n(CCN4CCN(C(=O)COc5cccc6c5CN(C5CCC(=O)NC5=O)C6=O)CC4)c13)CCC2 ; Cc1nn2c(c1-c1c(F)ccc3c(CCCOc4cccc5cc(F)ccc45)c(C(=O)O)n(CCN4CCN(C(=O)COc5cccc6c5CN(C5CCC(=O)NC5=O)C6=O)CC4)c13)CCC2]

tert-butyl 1-(2-(4-(2-((2-(2,6-dioxopiperidin-3-yl)-1-oxoisoindolin-4-yl)oxy)acetyl)piperazin-1-yl)ethyl)-6-fluoro-3-(3-((6-fluoronaphthalen-1-yl)oxy)propyl)-7-(2-methyl-5,6-dihydro-4H-pyrrolo[1,2-b]pyrazol-3-yl)-1H-indole-2-carboxylate (72.0 mg, crude) was dissolved in dry DCM (0.40 mL) under argon atmosphere and TFA (0.40 mL, 5.224 mmol) was added. The reaction (monitored with LCMS) was stirred for 18 h in room temperature under argon. After complete consumption of the starting material, reaction mixture was diluted with DCM and washed with brine and water. Solvent was evaporated under reduced pressure and dissolved in DMSO and purified with preparative HPLC (H₂O:MeCN+0.1% FA) to give 1-(2-(4-(2-((2-(2,6-dioxopiperidin-3-yl)-1-oxoisoindolin-4-yl)oxy)acetyl)piperazin-1-yl)ethyl)-6-fluoro-3-(3-((6-fluoronaphthalen-1-yl)oxy)propyl)-7-(2-methyl-5,6-dihydro-4H-pyrrolo[1,2-b]pyrazol-3-yl)-1H-indole-2-carboxylic acid (36.3 mg, 0.040 mmol, 53% over two steps) as white powder.

LCMS (ESI+): m/z 914.08 [M+H]⁺

¹H NMR (500 MHz, DMSO, 353K) δ 12.80 (s, 1H), 10.65 (s, 1H), 8.25 (dd, J=9.2, 5.8 Hz, 1H), 7.72 (dd, J=8.7, 5.4 Hz, 1H), 7.60 (dd, J=10.4, 2.6 Hz, 1H), 7.51-7.39 (m, 3H), 7.39-7.29 (m, 2H), 7.15 (d, J=8.1 Hz, 1H), 7.04-6.95 (m, 1H), 6.93-6.84 (m, 1H), 5.06 (dd, J=13.0, 5.2 Hz, 1H), 4.89 (s, 2H), 4.47-4.30 (m, 4H), 4.27 (t, J=6.3 Hz, 2H), 4.19-4.04 (m, 2H), 3.39-3.27 (m, 6H), 2.89 (ddd, J=17.2, 13.4, 5.5 Hz, 1H), 2.80-2.69 (m, 2H), 2.64-2.55 (m, 2H), 2.48-2.40 (m, 2H), 2.31-2.21 (m, 2H), 2.16-2.04 (m, 7H), 2.00 (s, 3H).

### Example 61. 1-{2-[4-(2-{[2-(2,6-dioxopiperidin-3-yl)-1-oxo-2,3-dihydro-1H-isoindol-4-yl]oxy}acetyl)piperazin-1-yl]ethyl}-6-fluoro-3-{3-[(6-fluoronaphthalen-1-yl)oxy]propyl}-7-{2-methyl-4H,6H,7H-pyrazolo[3,2-c][1,4]oxazin-3-yl}-1H-indole-2-carboxylic acid (226)

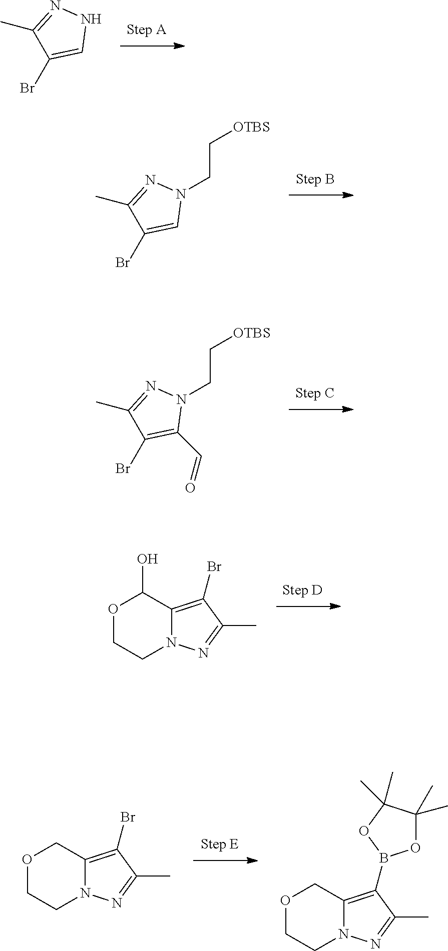

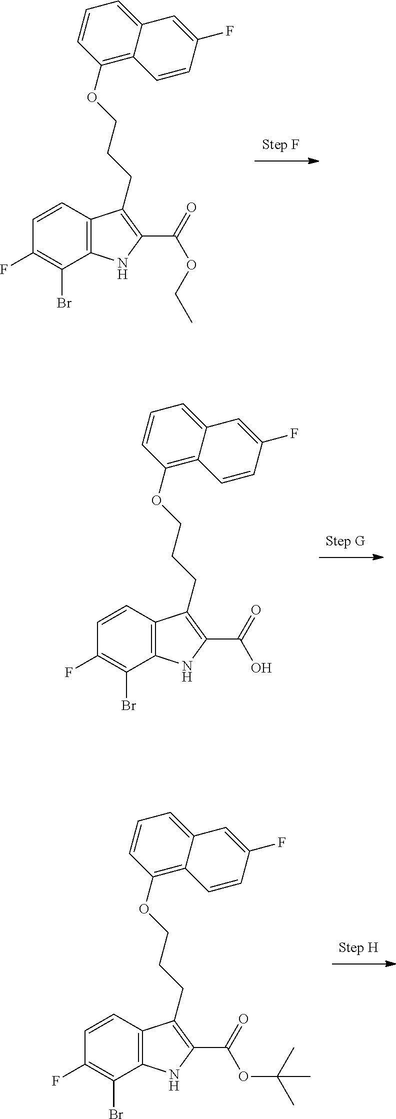 [SMILES: CCOC(=O)c1[nH]c2c(Br)c(F)ccc2c1CCCOc1cccc2cc(F)ccc12 ; O=C(O)c1[nH]c2c(Br)c(F)ccc2c1CCCOc1cccc2cc(F)ccc12 ; CC(C)(C)OC(=O)c1[nH]c2c(Br)c(F)ccc2c1CCCOc1cccc2cc(F)ccc12]

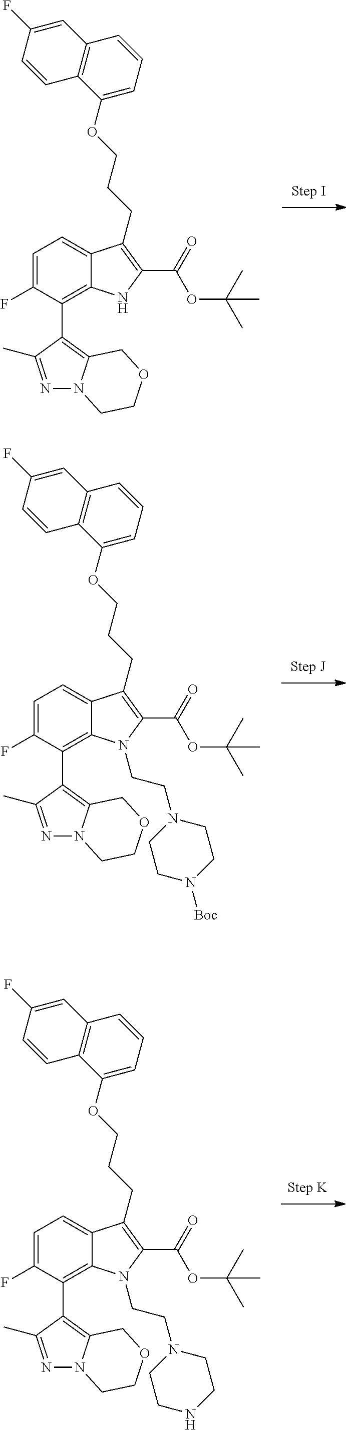

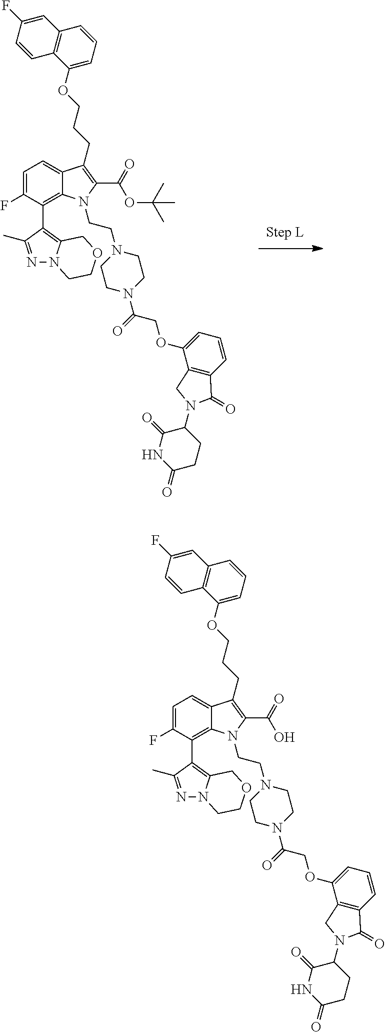 [SMILES: Cc1nn2c(c1-c1c(F)ccc3c(CCCOc4cccc5cc(F)ccc45)c(C(=O)OC(C)(C)C)n(CCN4CCN(C(=O)COc5cccc6c5CN(C5CCC(=O)NC5=O)C6=O)CC4)c13)COCC2 ; Cc1nn2c(c1-c1c(F)ccc3c(CCCOc4cccc5cc(F)ccc45)c(C(=O)O)n(CCN4CCN(C(=O)COc5cccc6c5CN(C5CCC(=O)NC5=O)C6=O)CC4)c13)COCC2]

### Step A

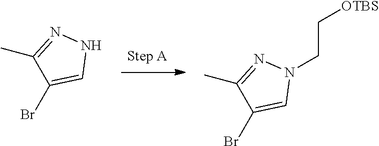

To a stirred solution of 4-bromo-3-methyl-1H-pyrazole (5 g, 31.1 mmol) in acetonitrile (120 mL) was added Cs₂CO₃ (15.2 g, 5.10 mmol), followed by a solution of (2-bromoethoxy)-tert-butyldimethylsilane (0.562 mL, 3.57 mmol) in acetonitrile (30 mL) drop wise at ambient temperature under nitrogen. The reaction mixture was stirred at RT for 16 h, and then reaction mixture was filtered through Celite bed, washing with Et₂O (50 mL). The filtrate was concentrated and the resultant residue was purified by column chromatography (SiO₂, EtOAc:hexane, 0-10%) to get 4-bromo-1-(2-((tert-butyldimethylsilyl)oxy)ethyl)-3-methyl-1H-pyrazole (6 g, 18.8 mmol, 60.5%) as colorless oil.

LCMS (ESI+): m/z 319.1 [M+H]⁺

### Step B

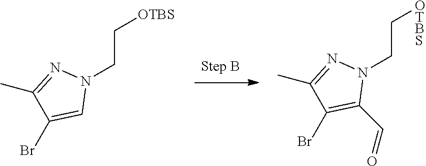 [SMILES: Cc1nn(CCO[Si](C)(C)C(C)(C)C)cc1Br ; Cc1nn(CCO[Si](C)(C)C(C)(C)C)c(C=O)c1Br]

To a well stirred solution of 2M LDA (18.8 mL, 37.6 mmol) in THE was added a solution of 4-bromo-1-(2-((tert-butyldimethylsilyl)oxy)ethyl)-3-methyl-1H-pyrazole (6 g, 18.8 mmol) in THE (12 mL) drop wise at −78° C. under nitrogen. The reaction mixture was allowed to stir at same temperature for 30 min. To the mixture was added a solution of DMF (2.6 ml, 33.85 mmol) in THE (10 ml) drop wise at −78° C. and the resulting reaction mixture was stirred for another 1 h at same temperature. After complete consumption of the starting material the excess LDA was quenched by saturated NH₄Cl solution and the reaction mixture was diluted with EtOAc, washed successively with water and brine, the organic layer was dried over Na₂SO₄ and evaporated under reduced pressure to get the crude compound, which was then purified by column chromatography (SiO₂, EtOAc:hexane, 5-10%) to get 4-bromo-1-(2-((tert-butyldimethylsilyl)oxy)ethyl)-3-methyl-1H-pyrazole-5-carbaldehyde (3 g, 8.64 mmol, 46%) as white sticky solid.

LCMS (ESI+): m/z 349.0 [M+H]⁺

### Step C

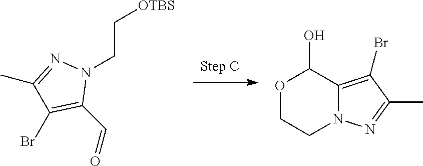 [SMILES: Cc1nn(CCO[Si](C)(C)C(C)(C)C)c(C=O)c1Br ; Cc1nn2c(c1Br)C(O)OCC2]

To a stirred solution of 4-bromo-1-(2-((tert-butyldimethylsilyl)oxy)ethyl)-3-methyl-1H-pyrazole-5-carbaldehyde (3 g, 8.64 mmol) in 2-methyl THE (15 mL) and water (15 mL) was added TFA (30 mL) dropwise at RT under nitrogen. The reaction mixture was allowed to stir at RT for 1 h. After complete consumption of the starting material the volatiles were evaporated under reduced pressure, crude mixture was diluted with EtOAc, washed successively with saturated NaHCO₃ solution, water and brine, the organic layer was dried over Na₂SO₄ and evaporated under reduced pressure to get 4 g of crude 3-bromo-2-methyl-6,7-dihydro-4H-pyrazolo[5,1-c][1,4]oxazin-4-ol as white solid which was then directly used for next step without further purification.

LCMS (ESI+): m/z 234.8 [M+H]⁺

### Step D

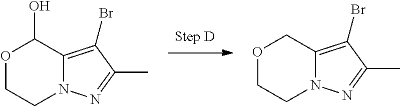

To a well stirred solution of crude 3-bromo-2-methyl-6,7-dihydro-4H-pyrazolo[5,1-c][1,4]oxazin-4-ol (3 g, 12.8 mmol) in DCM (50 mL) was added TFA (8.8 mL, 77.2 mmol), and Et₃SiH (4.48 mL, 38.6 mmol) successively at 0° C. under nitrogen. The reaction mixture was allowed to stir at 0° C. for 1 h. After that again TFA (4.4 mL, 38.6 mmol), and Et₃SiH (2.3 mL, 19.4 mmol) were added successively and it was stirred for 16 h at RT. After complete consumption of starting material (monitored by TLC and LCMS) the reaction mixture was evaporated under reduced pressure to get the crude, which was diluted with DCM, washed successively with saturated NaHCO₃ solution and brine. The organic layer was dried over Na₂SO₄ and evaporated under reduced pressure to get the crude compound, which was then purified by column chromatography (SiO2, EtOAc:hexane, 30-40%) to get 3-bromo-2-methyl-6,7-dihydro-4H-pyrazolo[5,1-c][1,4]oxazine (800 mg, 3.68 mmol, 42% over two steps) as off white sticky solid.

### Step E

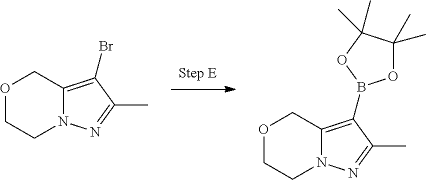 [SMILES: Cc1nn2c(c1Br)COCC2 ; Cc1nn2c(c1B1OC(C)(C)C(C)(C)O1)COCC2]

To a well stirred solution of 3-bromo-2-methyl-6,7-dihydro-4H-pyrazolo[5,1-c][1,4]oxazine (1.2 g, 5.6 mmol) and 2-isopropoxy-4,4,5,5-tetramethyl-1,3,2-dioxaborolane (2.2 mL, 16.7 mmol) in THE (120 mL), was added n-butyllithium (1.7 M, 7.9 mL, 13.4 mmol) at −78° C. under argon. The resulting mixture was then allowed to stir at −78° C. for 2 h. The mixture was slowly warmed to RT and was stirred for another 30 min. After complete consumption of the starting material (monitored by TLC and LCMS) the excess n-butyllithium was quenched by addition of saturated NH₄Cl and it was diluted with EtOAc, washed successively with water and brine. The organic layer was dried over Na₂SO₄ and evaporated under reduced pressure to get the crude compound, which was then purified by column chromatography (SiO2, EtOAc:hexane, 10-20%) to get 2-methyl-3-(4,4,5,5-tetramethyl-1,3,2-dioxaborolan-2-yl)-6,7-dihydro-4H-pyrazolo[5,1-c][1,4]oxazine (610 mg, 2.31 mmol, 41%) as white sticky solid.

LCMS (ESI+): m/z 265.1 [M+H]⁺

### Step F

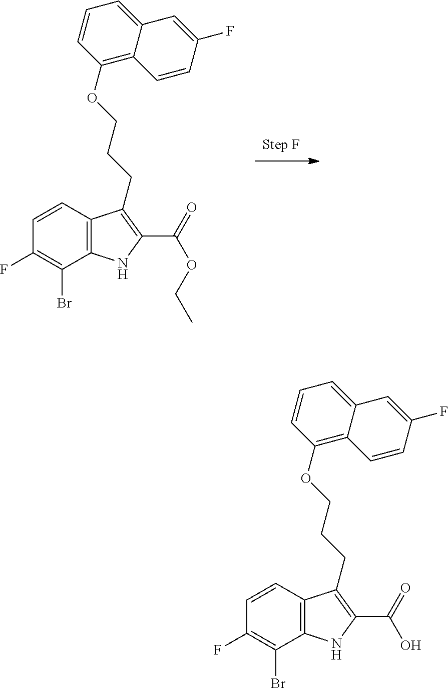 [SMILES: CCOC(=O)c1[nH]c2c(Br)c(F)ccc2c1CCCOc1cccc2cc(F)ccc12 ; O=C(O)c1[nH]c2c(Br)c(F)ccc2c1CCCOc1cccc2cc(F)ccc12]

Ethyl 7-bromo-6-fluoro-3-(3-((6-fluoronaphthalen-1-yl)oxy)propyl)-1H-indole-2-carboxylate (3.3 g, 6.8 mmol) was dissolved in EtOH (40 mL) and a solution of NaOH (1.3 g, 33.9 mmol) in water (6 mL) was added to it. The mixture was heated under reflux for 16 h. After complete consumption of the starting material (monitored by TLC and LCMS) the reaction mixture was cooled down to room temperature. The solvents were evaporated under reduced pressure to get the crude reaction mixture. It was then diluted with water, extracted with EtOAc. Aqueous layer was carefully acidified using 1 (N) HCl to pH=3, extracted with DCM (3×), dried over Na₂SO₄, filtered, and concentrated in vacuo to afford 7-bromo-6-fluoro-3-(3-((6-fluoronaphthalen-1-yl)oxy)propyl)-1H-indole-2-carboxylic acid (2.8 g, 6.1 mmol, 90%) as brown solid.

LCMS (ESI+): m/z 458.2 [M+H]⁺

### Step G

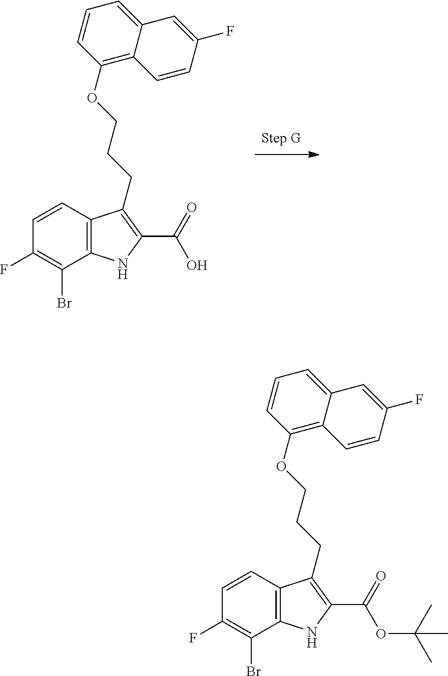 [SMILES: O=C(O)c1[nH]c2c(Br)c(F)ccc2c1CCCOc1cccc2cc(F)ccc12 ; CC(C)(C)OC(=O)c1[nH]c2c(Br)c(F)ccc2c1CCCOc1cccc2cc(F)ccc12]

7-bromo-6-fluoro-3-(3-((6-fluoronaphthalen-1-yl)oxy)propyl)-1H-indole-2-carboxylic acid (2.8 g, 6.1 mmol) was suspended in toluene (40 mL) and the mixture was heated to reflux under nitrogen. N,N-dimethylformamide di-tert-butyl acetal (9.9 mL, 48.8 mmol) was added drop wise to the refluxing mixture and stirred for an additional 16 h under nitrogen. After 16 h next 4.9 mL (24.4 mmol) of N,N-dimethylformamide di-tert-butyl acetal was added to it and the reaction was continued for another 8 h. After complete consumption of the stating material (monitored by TLC and LCMS) reaction mixture was diluted with EtOAc, washed successively with NaHCO₃ (sat), water and brine. The organic layer was dried over Na₂SO₄ and evaporated under reduced pressure to get the crude compound, which was then purified by column chromatography (SiO₂, 10% EtOAc in DCM) to get tert-butyl 7-bromo-6-fluoro-3-(3-((6-fluoronaphthalen-1-yl)oxy)propyl)-1H-indole-2-carboxylate (2.4 g, 4.65 mmol, 76.19%) as brown gummy solid.

### Step H

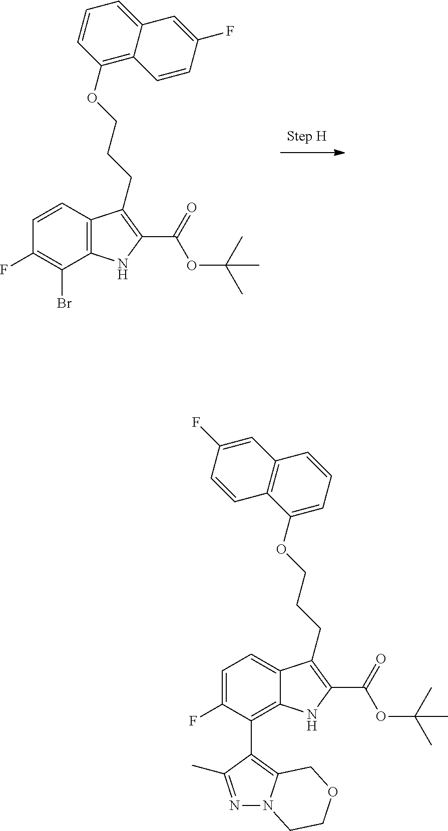 [SMILES: CC(C)(C)OC(=O)c1[nH]c2c(Br)c(F)ccc2c1CCCOc1cccc2cc(F)ccc12 ; Cc1nn2c(c1-c1c(F)ccc3c(CCCOc4cccc5cc(F)ccc45)c(C(=O)OC(C)(C)C)[nH]c13)COCC2]

To a stirred solution of tert-butyl 7-bromo-6-fluoro-3-(3-((6-fluoronaphthalen-1-yl)oxy)propyl)-1H-indole-2-carboxylate (600 mg, 1.2 mmol) in dioxane (8 mL) and water (2 mL) were added 2-methyl-3-(4,4,5,5-tetramethyl-1,3,2-dioxaborolan-2-yl)-6,7-dihydro-4H-pyrazolo[5,1-c][1,4]oxazine (613 mg, 2.3 mmol) followed by potassium phosphate (739 mg, 3.5 mmol). The mixture was deoxygenated with argon and to it was added [1,1′-bis(di-tert-butylphosphino)ferrocene]dichloropalladium(II) (151 mg, 0.233 mmol) under argon atmosphere. Then the reaction mixture was heated under reflux for 16 h. After complete consumption of the starting material (monitored by TLC and LCMS) the reaction mixture was filtered over Celite pad and the solvents were evaporated under reduced pressure. The residues were diluted with EtOAc, washed successively with water and brine. The organic layer was dried over Na₂SO₄ and evaporated under reduced pressure to get the crude compound, which was then purified by column chromatography (SiO₂, 30% EtOAc in DCM) to get tert-butyl 6-fluoro-3-(3-((6-fluoronaphthalen-1-yl)oxy)propyl)-7-(2-methyl-6,7-dihydro-4H-pyrazolo[5,1-c][1,4]oxazin-3-yl)-1H-indole-2-carboxylate (550 mg, 0.958 mmol, 82%) as brown solid.

LCMS (ESI+): m/z 574.0 [M+H]⁺

### Step I

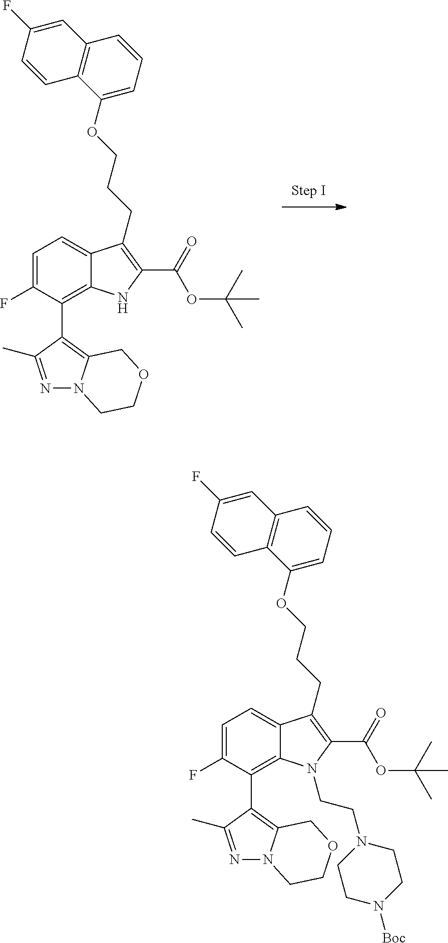 [SMILES: Cc1nn2c(c1-c1c(F)ccc3c(CCCOc4cccc5cc(F)ccc45)c(C(=O)OC(C)(C)C)[nH]c13)COCC2 ; Cc1nn2c(c1-c1c(F)ccc3c(CCCOc4cccc5cc(F)ccc45)c(C(=O)OC(C)(C)C)n(CCN4CCN(C(=O)OC(C)(C)C)CC4)c13)COCC2]

To a well stirred solution of get tert-butyl 6-fluoro-3-(3-((6-fluoronaphthalen-1-yl)oxy)propyl)-7-(2-methyl-6,7-dihydro-4H-pyrazolo[5,1-c][1,4]oxazin-3-yl)-1H-indole-2-carboxylate (550 mg, 0.96 mmol) in DMF (6 mL) was added tert-butyl 4-(2-chloroethyl)piperazine-1-carboxylate (309 mg, 1.25 mmol) followed by Cs₂CO₃ (467 mg, 1.4 mmol) in DMF (5 mL) and the mixture was allowed to stir at 90° C. for 16 h under nitrogen. After complete consumption of the starting material (monitored by TLC and LCMS) the reaction mixture was diluted with EtOAc, washed successively with water and brine. The organic layer was dried over Na₂SO₄ and evaporated under reduced pressure to get the crude compound, which was then purified by column chromatography (SiO₂, 40% EtOAc in DCM) to get tert-butyl 1-(2-(4-(tert-butoxycarbonyl)piperazin-1-yl)ethyl)-6-fluoro-3-(3-((6-fluoronaphthalen-1-yl)oxy)propyl)-7-(2-methyl-6,7-dihydro-4H-pyrazolo[5,1-c][1,4]oxazin-3-yl)-1H-indole-2-carboxylate (520 mg, 0.662 mmol, 69%) as white solid.

LCMS (ESI+): m/z 786.4 [M+H]⁺

### Step J

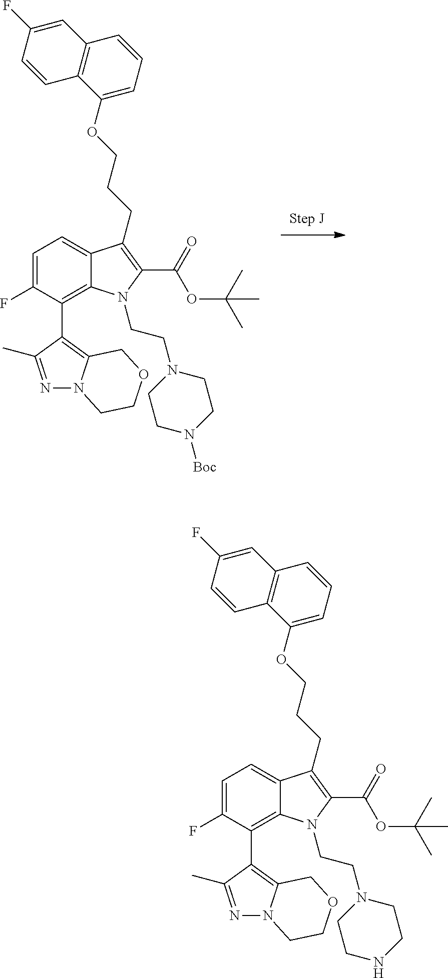 [SMILES: Cc1nn2c(c1-c1c(F)ccc3c(CCCOc4cccc5cc(F)ccc45)c(C(=O)OC(C)(C)C)n(CCN4CCN(C(=O)OC(C)(C)C)CC4)c13)COCC2 ; Cc1nn2c(c1-c1c(F)ccc3c(CCCOc4cccc5cc(F)ccc45)c(C(=O)OC(C)(C)C)n(CCN4CCNCC4)c13)COCC2]

To a solution of tert-butyl 1-(2-(4-(tert-butoxycarbonyl)piperazin-1-yl)ethyl)-6-fluoro-3-(3-((6-fluoronaphthalen-1-yl)oxy)propyl)-7-(2-methyl-6,7-dihydro-4H-pyrazolo[5,1-c][1,4]oxazin-3-yl)-1H-indole-2-carboxylate (520 mg, 0.662 mmol) in DCM (20 mL) was added 4 (M) HCl in dioxane (10 mL) drop wise at 0° C. and the mixture was allowed to stir for 2 h under nitrogen at same temperature. After complete consumption of the starting material (monitored by TLC and LCMS), the reaction mixture was quenched by drop wise addition of cold 1M NaOH solution at 0° C. to maintain the pH˜7 to 8. The aqueous layer was extracted with DCM (3×). The combined organics layers were dried over Na₂SO₄ and concentrated in vacuo to get the crude compound which was then purified by column chromatography (amine SiO2, 70% EtOAc in DCM) to get tert-butyl 6-fluoro-3-(3-((6-fluoronaphthalen-1-yl)oxy)propyl)-7-(2-methyl-6,7-dihydro-4H-pyrazolo[5,1-c][1,4]oxazin-3-yl)-1-(2-(piperazin-1-yl)ethyl)-1H-indole-2-carboxylate (190 mg, 0.277 mmol, 42.92%) as white solid.

LCMS (ESI+): m/z 686.5 [M+H]⁺

### Step K

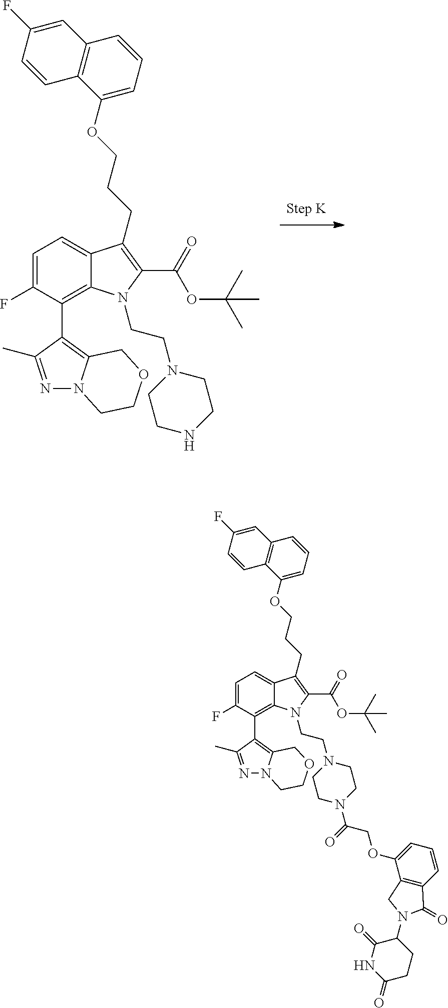 [SMILES: Cc1nn2c(c1-c1c(F)ccc3c(CCCOc4cccc5cc(F)ccc45)c(C(=O)OC(C)(C)C)n(CCN4CCNCC4)c13)COCC2 ; Cc1nn2c(c1-c1c(F)ccc3c(CCCOc4cccc5cc(F)ccc45)c(C(=O)OC(C)(C)C)n(CCN4CCN(C(=O)COc5cccc6c5CN(C5CCC(=O)NC5=O)C6=O)CC4)c13)COCC2]

To a well stirred solution of 2-{[2-(2,6-dioxopiperidin-3-yl)-1-oxo-2,3-dihydro-1H-isoindol-4-yl]oxy}acetic acid (27.8 mg, 0.087 mmol) and tert-butyl 6-fluoro-3-(3-((6-fluoronaphthalen-1-yl)oxy)propyl)-7-(2-methyl-6,7-dihydro-4H-pyrazolo[5,1-c][1,4]oxazin-3-yl)-1-(2-(piperazin-1-yl)ethyl)-1H-indole-2-carboxylate (50.0 mg, 0.073 mmol) in DMF (2.0 mL) was added DIPEA (0.063 mL, 0.365 mmol) and HATU (55.4 mg, 0.146 mmol) and the mixture was allowed to stir under nitrogen for 1 h at RT. After complete consumption of the starting material (monitored by LCMS), then the reaction mixture diluted with DCM and washed twice with brine. Organic layer was dried over MgSO₄ and evaporated under reduced pressure. The product tert-butyl 1-(2-(4-(2-((2-(2,6-dioxopiperidin-3-yl)-1-oxoisoindolin-4-yl)oxy)acetyl)piperazin-1-yl)ethyl)-6-fluoro-3-(3-((6-fluoronaphthalen-1-yl)oxy)propyl)-7-(2-methyl-6,7-dihydro-4H-pyrazolo[5,1-c][1,4]oxazin-3-yl)-1H-indole-2-carboxylate (55.0 mg, crude) was obtained as brown oil, which was used for the next step without further purification.

LCMS (ESI+): m/z 985.6 [M+H]⁺

### Step L

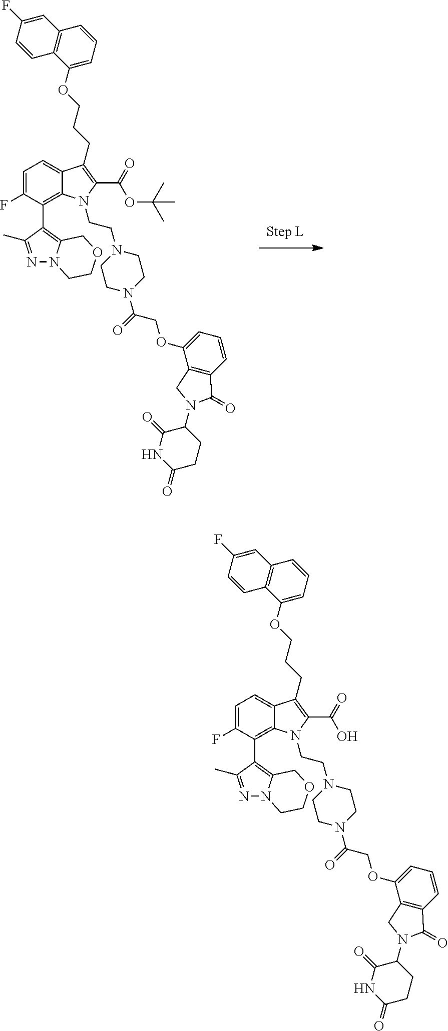 [SMILES: Cc1nn2c(c1-c1c(F)ccc3c(CCCOc4cccc5cc(F)ccc45)c(C(=O)OC(C)(C)C)n(CCN4CCN(C(=O)COc5cccc6c5CN(C5CCC(=O)NC5=O)C6=O)CC4)c13)COCC2 ; Cc1nn2c(c1-c1c(F)ccc3c(CCCOc4cccc5cc(F)ccc45)c(C(=O)O)n(CCN4CCN(C(=O)COc5cccc6c5CN(C5CCC(=O)NC5=O)C6=O)CC4)c13)COCC2]

To a 55.0 mg crude tert-butyl 1-(2-(4-(2-((2-(2,6-dioxopiperidin-3-yl)-1-oxoisoindolin-4-yl)oxy)acetyl)piperazin-1-yl)ethyl)-6-fluoro-3-(3-((6-fluoronaphthalen-1-yl)oxy)propyl)-7-(2-methyl-6,7-dihydro-4H-pyrazolo[5,1-c][1,4]oxazin-3-yl)-1H-indole-2-carboxylate was added dry DCM (0.500 mL) followed by TFA (0.5 mL, 6.529 mmol) and a mixture was stirred for 18 h at RT. After a reaction was completed (monitored by LCMS), solvents were evaporated and residues were dissolved in DMSO and purified by preparative HPLC (H₂O:MeCN+0.1% FA) to give 1-(2-(4-(2-((2-(2,6-dioxopiperidin-3-yl)-1-oxoisoindolin-4-yl)oxy)acetyl)piperazin-1-yl)ethyl)-6-fluoro-3-(3-((6-fluoronaphthalen-1-yl)oxy)propyl)-7-(2-methyl-6,7-dihydro-4H-pyrazolo[5,1-c][1,4]oxazin-3-yl)-1H-indole-2-carboxylic acid (26.2 mg, 0.028 mmol, 50.5%) as a white solid.

LCMS (ESI+): m/z 930.3 [M+H]⁺

¹H NMR (500 MHz, DMSO) δ 13.27 (s, 1H), 10.96 (s, 1H), 8.23 (dd, J=9.2, 5.9 Hz, 1H), 7.76 (dd, J=8.7, 5.4 Hz, 1H), 7.64 (dd, J=10.4, 2.7 Hz, 1H), 7.46-7.40 (m, 3H), 7.36 (td, J=8.9, 2.7 Hz, 1H), 7.31 (d, J=7.4 Hz, 1H), 7.10 (d, J=8.2 Hz, 1H), 7.02 (t, J=9.2 Hz, 1H), 6.87 (p, J=4.8 Hz, 1H), 5.10 (dd, J=13.3, 5.1 Hz, 1H), 4.98-4.88 (m, 2H), 4.56 (s, 2H), 4.46-4.33 (m, 2H), 4.29-4.04 (m, 8H), 3.29-3.24 (m, 4H), 2.90 (ddd, J=17.3, 13.6, 5.5 Hz, 1H), 2.62-2.52 (m, 2H), 2.47-2.39 (m, 1H), 2.20 (p, J=6.3 Hz, 2H), 2.13-1.93 (m, 11H).

### Example 62. 6-chloro-1-{2-[4-(2-{4-[(2,6-dioxopiperidin-3-yl)carbamoyl]-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (2681

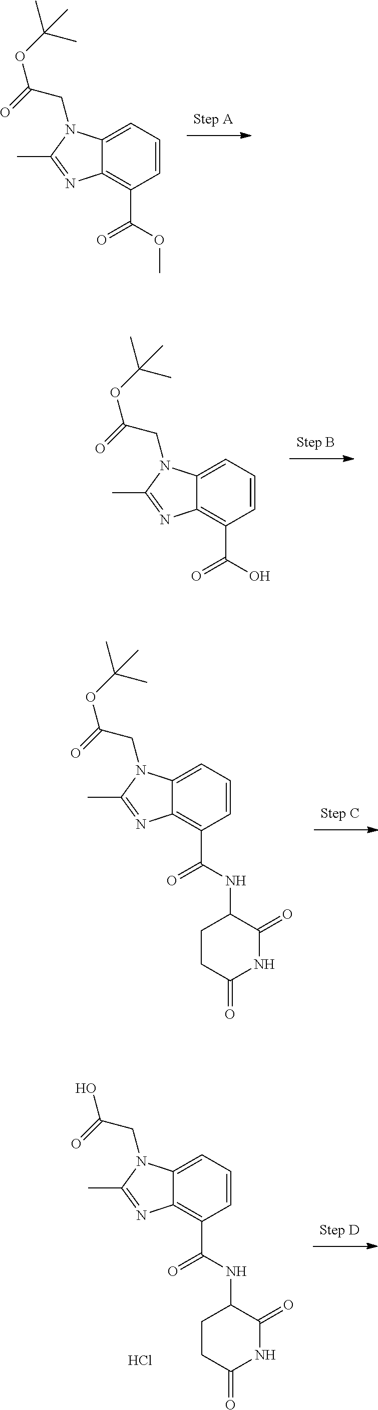 [SMILES: COC(=O)c1cccc2c1nc(C)n2CC(=O)OC(C)(C)C ; Cc1nc2c(C(=O)O)cccc2n1CC(=O)OC(C)(C)C ; Cc1nc2c(C(=O)NC3CCC(=O)NC3=O)cccc2n1CC(=O)OC(C)(C)C ; Cc1nc2c(C(=O)NC3CCC(=O)NC3=O)cccc2n1CC(=O)O ; Cl]

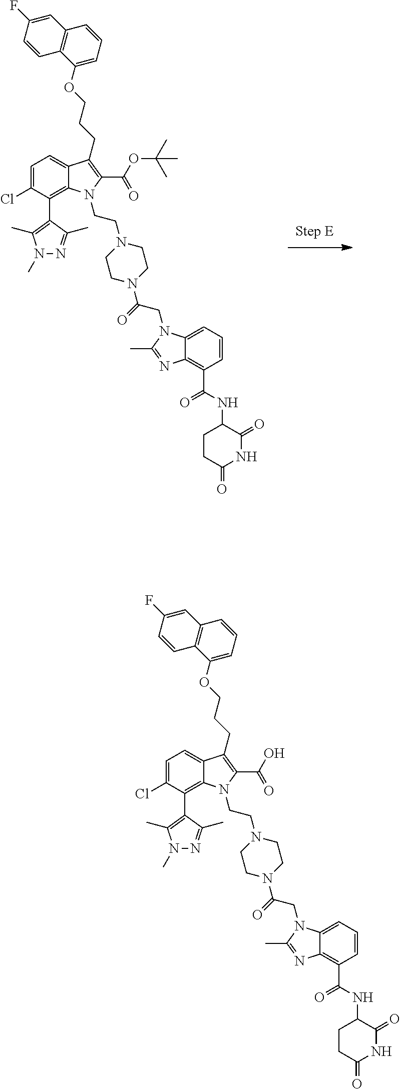 [SMILES: Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)cccc54)CC3)c12 ; Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cccc54)CC3)c12]

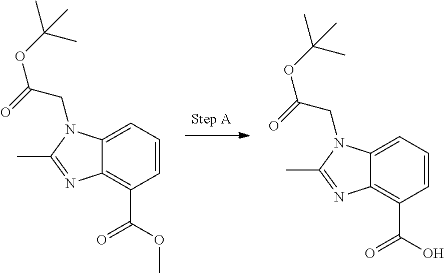 [SMILES: COC(=O)c1cccc2c1nc(C)n2CC(=O)OC(C)(C)C ; Cc1nc2c(C(=O)O)cccc2n1CC(=O)OC(C)(C)C]

To a solution of methyl 1-[2-(tert-butoxy)-2-oxoethyl]-2-methyl-1H-1,3-benzodiazole-4-carboxylate (152.0 mg, 0.499 mmol) in H₂O (0.500 mL) and MeCN (5.0 mL) was added LiBr (867.4 mg, 9.989 mmol) and Et₃N (0.695 mL, 4.994 mmol). The mixture was stirred for 4 days at RT. The crude was concentrated in vacuo and purified by reverse phase flash chromatography (H₂O:MeCN+0.1% FA) to get 1-[2-(tert-butoxy)-2-oxoethyl]-2-methyl-1H-1,3-benzodiazole-4-carboxylic acid (75.0 mg, 0.233 mmol, 47%) as a yellow solid.

LCMS (ESI+): m/z 291.0 [M+H]⁺

### Step B

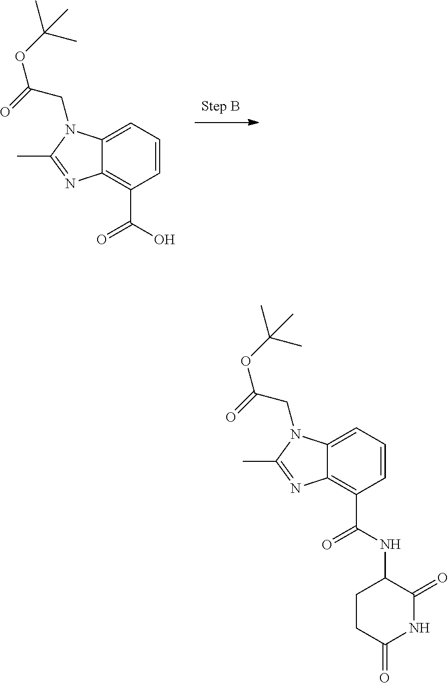 [SMILES: Cc1nc2c(C(=O)O)cccc2n1CC(=O)OC(C)(C)C ; Cc1nc2c(C(=O)NC3CCC(=O)NC3=O)cccc2n1CC(=O)OC(C)(C)C]

To a solution of 1-[2-(tert-butoxy)-2-oxoethyl]-2-methyl-1H-1,3-benzodiazole-4-carboxylic acid (100.0 mg, 0.344 mmol) and 3-aminopiperidine-2,6-dione hydrochloride (68.0 mg, 0.413 mmol) in dry DMF (2.0 mL) was added DIPEA (0.300 mL, 1.722 mmol) and HATU (196.5 mg, 0.517 mmol). The mixture was stirred for 18 h at RT. After this time, the crude was concentrated in vacuo and the residues were dissolved in DCM and purified by flash chromatography (SiO₂, MeOH:DCM, 0-50%) to isolate tert-butyl 2-{4-[(2,6-dioxopiperidin-3-yl)carbamoyl]-2-methyl-1H-1,3-benzodiazol-1-yl}acetate (74.0 mg, 0.185 mmol, 54%) as a yellow solid.

LCMS (ESI+): m/z 401.1 [M+H]⁺

### Step C

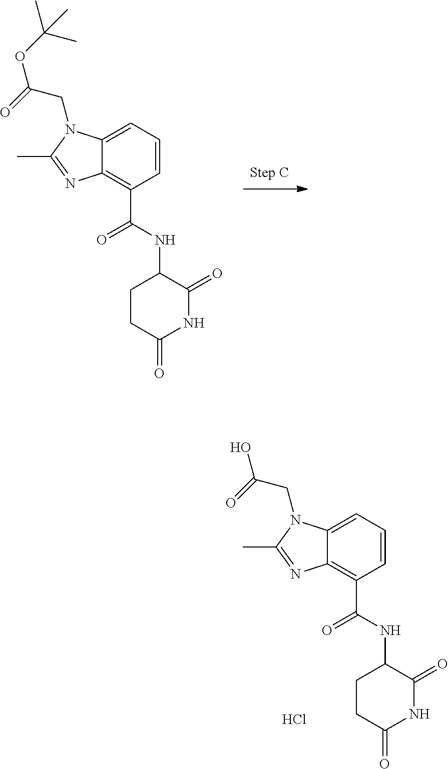 [SMILES: Cc1nc2c(C(=O)NC3CCC(=O)NC3=O)cccc2n1CC(=O)OC(C)(C)C ; Cc1nc2c(C(=O)NC3CCC(=O)NC3=O)cccc2n1CC(=O)O ; Cl]

To a solution of tert-butyl 2-{4-[(2,6-dioxopiperidin-3-yl)carbamoyl]-2-methyl-1H-1,3-benzodiazol-1-yl}acetate (64.5 mg, 0.161 mmol) in DCM (2.0 mL) was added TFA (1.0 mL, 13.059 mmol). The mixture was stirred for 18 h at RT. The crude was concentrated in vacuo and dissolved in H₂O. To the solution was added 1M HCl in water and it was evaporated. The product, 2-{4-[(2,6-dioxopiperidin-3-yl)carbamoyl]-2-methyl-1H-1,3-benzodiazol-1-yl}acetic acid hydrochloride (58.8 mg, 0.154 mmol, 96%) was isolated as beige solid.

LCMS (ESI+): m/z 344.8 [M+H]⁺

### Step D

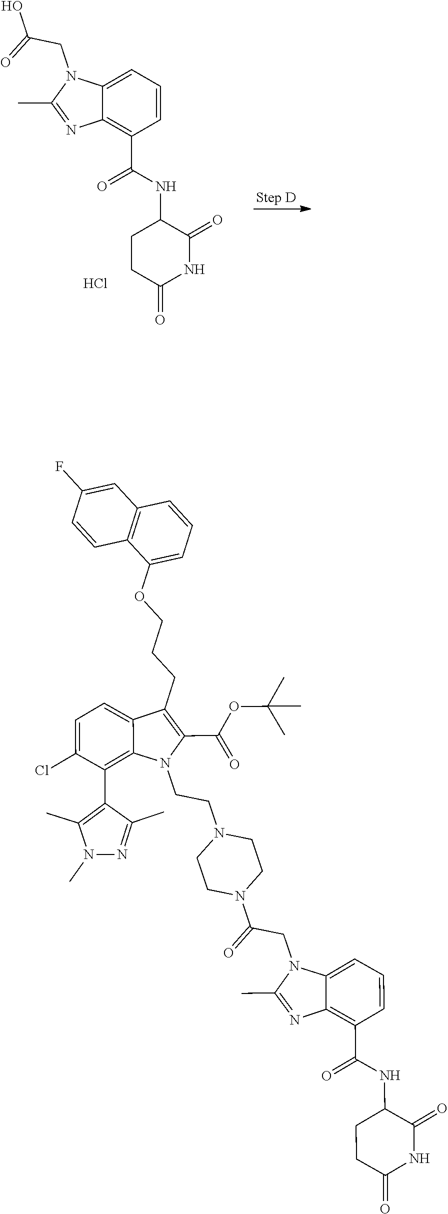 [SMILES: Cc1nc2c(C(=O)NC3CCC(=O)NC3=O)cccc2n1CC(=O)O ; Cl ; Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)cccc54)CC3)c12]

2-{4-[(2,6-Dioxopiperidin-3-yl)carbamoyl]-2-methyl-1H-1,3-benzodiazol-1-yl}acetic acid hydrochloride (37.3 mg, 0.098 mmol), tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (55.0 mg, 0.082 mmol) and HATU (62.0 mg, 0.163 mmol) were dissolved in dry DMF (2.0 mL) and to the mixture was added DIPEA (0.071 mL, 0.408 mmol). The reaction was stirred at RT for 2 h. The solvent was evaporated. Residues were dissolved in DCM and washed with brine. The organic layer was dried over Na₂SO₄, filtered and concentrated in vacuo to get tert-butyl 6-chloro-1-{2-[4-(2-{4-[(2,6-dioxopiperidin-3-yl)carbamoyl]-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (102.2 mg, crude) which was used without a further purification to next step.

LCMS (ESI+): m/z 1001.3 [M+H]⁺

### Step E

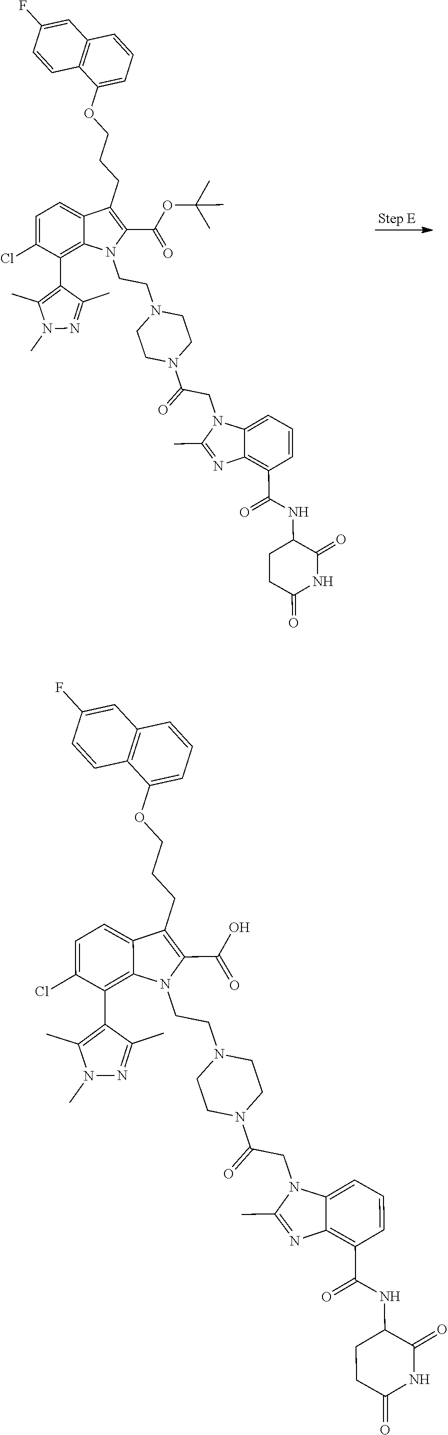 [SMILES: Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)cccc54)CC3)c12 ; Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cccc54)CC3)c12]

To a solution of tert-butyl 6-chloro-1-{2-[4-(2-{4-[(2,6-dioxopiperidin-3-yl)carbamoyl]-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (102.2 mg, crude) in DCM (1.0 mL) was added TFA (0.500 mL, 6.529 mmol). The mixture was stirred for 18 h at RT. The crude was concentrated in vacuo and purified by preparative HPLC (H₂O:MeCN+0.1% FA) to give 6-chloro-1-{2-[4-(2-{4-[(2,6-dioxopiperidin-3-yl)carbamoyl]-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (32.3 mg, 0.034 mmol, 35%) as a white solid.

LCMS (ESI+): m/z 944.3 [M+H]⁺

¹H NMR (500 MHz, DMSO, 353 K) δ 10.57 (s, 1H), 10.17 (d, J=7.2 Hz, 1H), 8.25 (dd, J=9.3, 5.9 Hz, 1H), 7.86 (dd, J=7.6, 1.1 Hz, 1H), 7.73 (d, J=8.5 Hz, 1H), 7.65-7.52 (m, 2H), 7.50-7.37 (m, 2H), 7.33 (td, J=8.9, 2.7 Hz, 1H), 7.28 (t, J=7.8 Hz, 1H), 7.23 (d, J=8.5 Hz, 1H), 6.88 (dd, J=5.5, 3.1 Hz, 1H), 5.21 (s, 2H), 4.86 (ddd, J=12.3, 7.2, 5.2 Hz, 1H), 4.37-4.14 (m, 4H), 3.78 (s, 3H), 3.45 (bs, 4H), 3.35-3.26 (m, 2H), 2.81 (ddd, J=18.1, 13.0, 5.5 Hz, 1H), 2.65-2.58 (m, 1H), 2.35-2.28 (m, 1H), 2.28-2.23 (m, 2H), 2.23-2.07 (m, 7H), 2.04 (d, J=5.2 Hz, 3H), 1.91 (s, 3H).

3 protons overlap with solvents in aliphatic area.

### Example 63. 6-Chloro-1-(2-(7-(2-((2-(2,6-dioxopiperidin-3-yl)-1-oxoisoindolin-4-yl)oxy)acetyl)-4,7-diazaspiro[2.5]octan-4-yl)ethyl)-3-(3-(naphthalen-1-yloxy)propyl)-7-(1,3,5-trimethyl-1H-pyrazol-4-yl)-1H-indole-2-carboxylic acid (292)

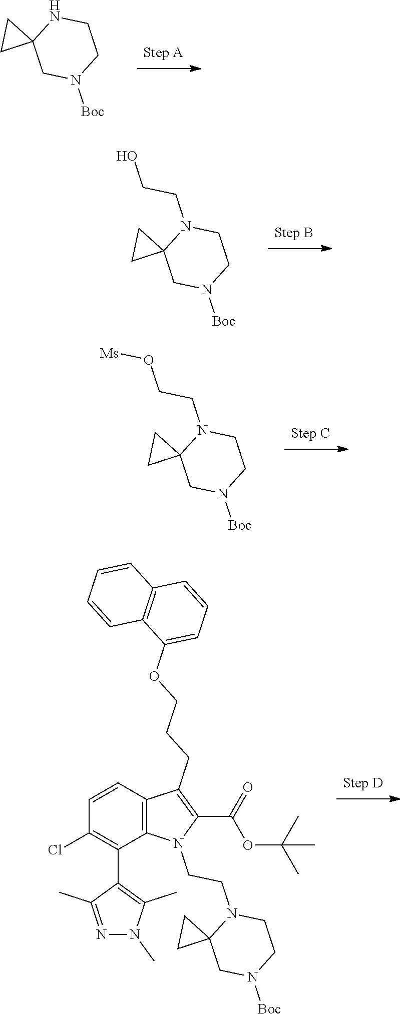 [SMILES: CC(C)(C)OC(=O)N1CCNC2(CC2)C1 ; CC(C)(C)OC(=O)N1CCN(CCO)C2(CC2)C1 ; CC(C)(C)OC(=O)N1CCN(CCOS(C)(=O)=O)C2(CC2)C1 ; Cc1nn(C)c(C)c1-c1c(Cl)ccc2c(CCCOc3cccc4ccccc34)c(C(=O)OC(C)(C)C)n(CCN3CCN(C(=O)OC(C)(C)C)CC34CC4)c12]

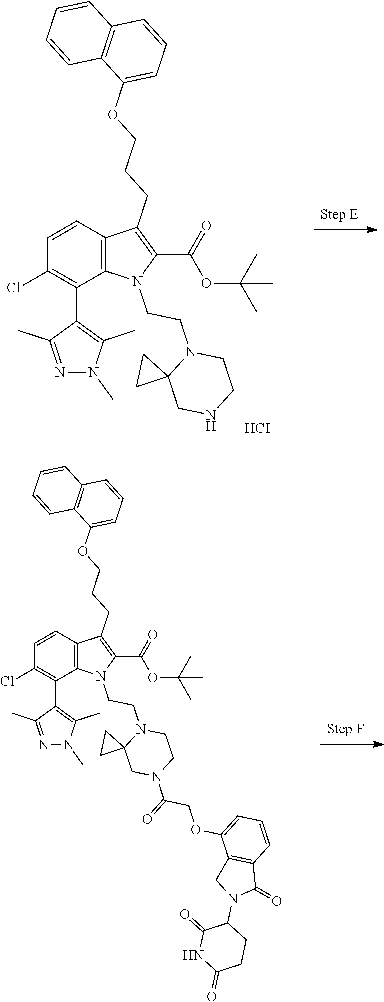

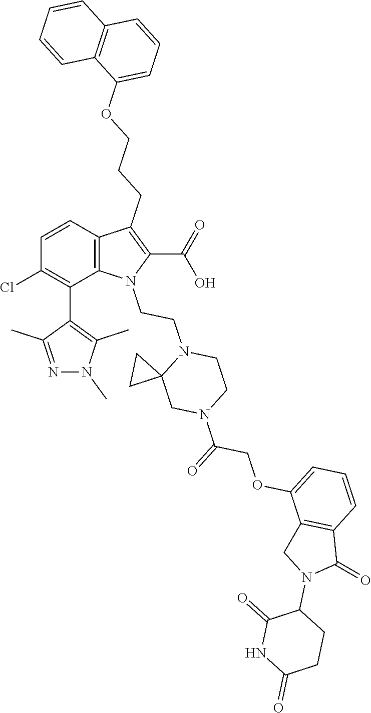 [SMILES: Cc1nn(C)c(C)c1-c1c(Cl)ccc2c(CCCOc3cccc4ccccc34)c(C(=O)O)n(CCN3CCN(C(=O)COc4cccc5c4CN(C4CCC(=O)NC4=O)C5=O)CC34CC4)c12]

### Step A

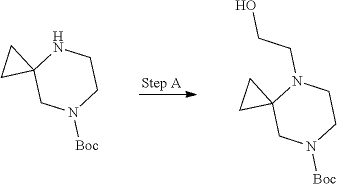 [SMILES: CC(C)(C)OC(=O)N1CCNC2(CC2)C1 ; CC(C)(C)OC(=O)N1CCN(CCO)C2(CC2)C1]

tert-Butyl 4,7-diazaspiro[2.5]octane-7-carboxylate (500.0 mg, 2.355 mmol), K₂CO₃ (976.5 mg, 7.066 mmol) were dissolved dry DMF (15.0 mL) and then 2-bromoethanol (0.835 mL, 11.776 mmol) was added. Reaction was stirred at 80° C. for 3 days. The reaction mixture was diluted in EtOAc and washed with brine. The organic layer was dried over Na₂SO₄, filtered and concentrated in vacuo to give tert-butyl 4-(2-hydroxyethyl)-4,7-diazaspiro[2.5]octane-7-carboxylate (695 mg, crude) as yellow oil, which was used directly in the next step without further purification.

LCMS (ESI+): m/z 257.1 [M+H]⁺

### Step B

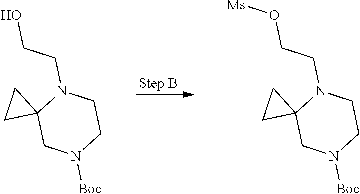 [SMILES: CC(C)(C)OC(=O)N1CCN(CCO)C2(CC2)C1 ; CC(C)(C)OC(=O)N1CCN(CCOS(C)(=O)=O)C2(CC2)C1]

tert-Butyl 4-(2-hydroxyethyl)-4,7-diazaspiro[2.5]octane-7-carboxylate (695.0 mg, 2.711 mmol) was dissolved in DCM (27.1 mL), Et₃N (0.566 mL, 4.067 mmol) and DMAP (33.1 mg, 0.271 mmol) were added and reaction mixture cooled to 0° C. Then MsCl (0.252 mL, 3.253 mmol) was added dropwise and reaction mixture was let to stir at room temperature for 2 h. The reaction mixture was diluted in EtOAc and washed with brine. The organic layer was dried over MgSO₄, filtered and concentrated in vacuo. The product tert-butyl 4-[2-(methanesulfonyloxy)ethyl]-4,7-diazaspiro[2.5]octane-7-carboxylate (649.0 mg, crude) was used directly in the next step without further purification.

LCMS (ESI+): m/z 335.3 [M+H]⁺

### Step C

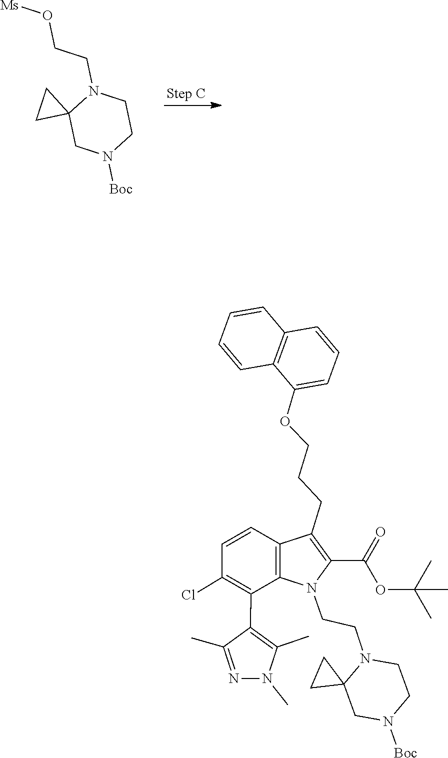 [SMILES: CC(C)(C)OC(=O)N1CCN(CCOS(C)(=O)=O)C2(CC2)C1 ; Cc1nn(C)c(C)c1-c1c(Cl)ccc2c(CCCOc3cccc4ccccc34)c(C(=O)OC(C)(C)C)n(CCN3CCN(C(=O)OC(C)(C)C)CC34CC4)c12]

tert-Butyl 4-[2-(methanesulfonyloxy)ethyl]-4,7-diazaspiro[2.5]octane-7-carboxylate (73.8 mg, crude), tert-butyl 6-chloro-3-[3-(naphthalen-1-yloxy)propyl]-7-(1,3,5-trimethyl-1H-pyrazol-4-yl)-1H-indole-2-carboxylate (100.0 mg, 0.184 mmol) and Cs₂CO₃ (179.6 mg, 0.551 mmol) was dissolved in dry DMF (4 mL) and stirred at 60° C. for 18 h. The reaction mixture was diluted in EtOAc and washed with brine. The organic layer was dried over Na₂SO₄, filtered and concentrated in vacuo to get tert-butyl 1-(2-{7-[(tert-butoxy)carbonyl]-4,7-diazaspiro[2.5]octan-4-yl}ethyl)-6-chloro-3-[3-(naphthalen-1-yloxy)propyl]-7-(1,3,5-trimethyl-1H-pyrazol-4-yl)-1H-indole-2-carboxylate (246.0 mg, crude) as a yellow oil, which was used to next step without further purification.

LCMS (ESI+): m/z 782.3 [M+H]⁺

### Step D

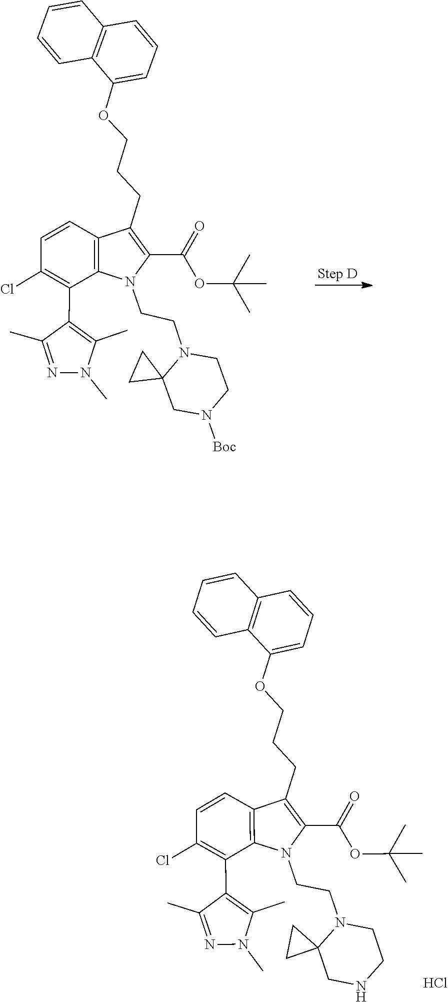

tert-Butyl 1-(2-{7-[(tert-butoxy)carbonyl]-4,7-diazaspiro[2.5]octan-4-yl}ethyl)-6-chloro-3-[3-(naphthalen-1-yloxy)propyl]-7-(1,3,5-trimethyl-1H-pyrazol-4-yl)-1H-indole-2-carboxylate (246.0 mg, 0.160 mmol) was dissolved in THE (3.2 mL), cooled to 0° C. and then 4M HCl in dioxane (1.0 mL, 4.009 mmol) was added dropwise. Reaction was stirred at room temperature for 18 h. Solvent was evaporated, dried under reduced pressure and 224 mg crude mixture of tert-butyl 6-chloro-1-(2-{4,7-diazaspiro[2.5]octan-4-yl}ethyl)-3-[3-(naphthalen-1-yloxy)propyl]-7-(1,3,5-trimethyl-1H-pyrazol-4-yl)-1H-indole-2-carboxylate hydrochloride was used directly in the next step.

LCMS (ESI+): m/z 682.33 [M+H]⁺

### Step E

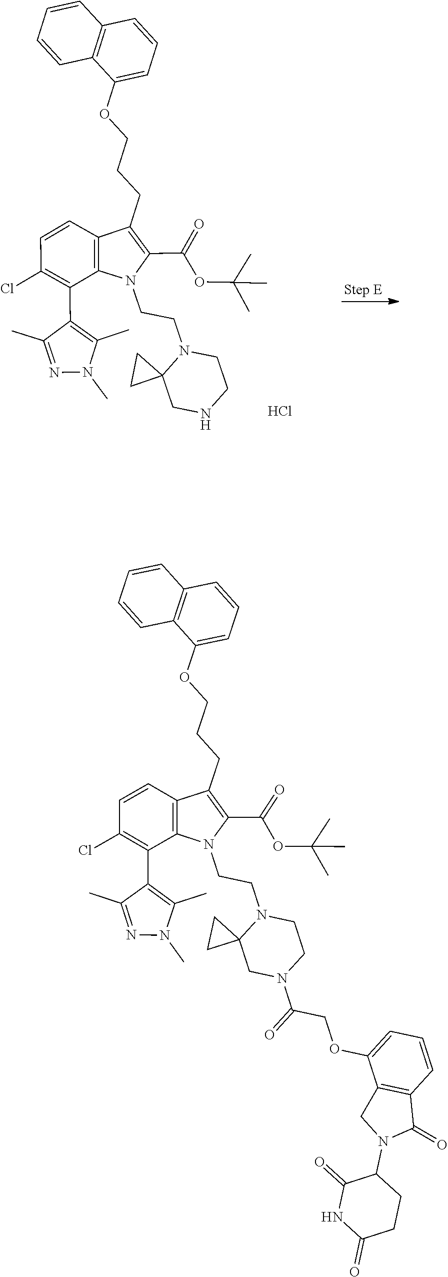 [SMILES: Cc1nn(C)c(C)c1-c1c(Cl)ccc2c(CCCOc3cccc4ccccc34)c(C(=O)OC(C)(C)C)n(CCN3CCNCC34CC4)c12 ; Cl ; Cc1nn(C)c(C)c1-c1c(Cl)ccc2c(CCCOc3cccc4ccccc34)c(C(=O)OC(C)(C)C)n(CCN3CCN(C(=O)COc4cccc5c4CN(C4CCC(=O)NC4=O)C5=O)CC34CC4)c12]

tert-Butyl 6-chloro-1-(2-{4,7-diazaspiro[2.5]octan-4-yl}ethyl)-3-[3-(naphthalen-1-yloxy)propyl]-7-(1,3,5-trimethyl-1H-pyrazol-4-yl)-1H-indole-2-carboxylate hydrochloride (75.0 mg, 0.104 mmol), 2-{[2-(2,6-dioxopiperidin-3-yl)-1-oxo-2,3-dihydro-1H-isoindol-4-yl]oxy}acetic acid (39.9 mg, 0.125 mmol) and HATU (79.4 mg, 0.209 mmol) was dissolved in dry DMF (1.0 mL) and to the mixture was added DIPEA (0.091 mL, 0.522 mmol). The reaction was stirred at room temperature for 30 min. The solvent was evaporated. Residues were dissolved in DCM and washed with brine. The organic layer was dried over Na₂SO₄, filtered and concentrated in vacuo to give a tert-butyl 6-chloro-1-{2-[7-(2-{[2-(2,6-dioxopiperidin-3-yl)-1-oxo-2,3-dihydro-1H-isoindol-4-yl]oxy}acetyl)-4,7-diazaspiro[2.5]octan-4-yl]ethyl}-3-[3-(naphthalen-1-yloxy)propyl]-7-(1,3,5-trimethyl-1H-pyrazol-4-yl)-1H-indole-2-carboxylate (85.0 mg, crude), which was used without a further purification to next step.

LCMS (ESI+): m/z 982.07 [M+H]⁺

### Step F

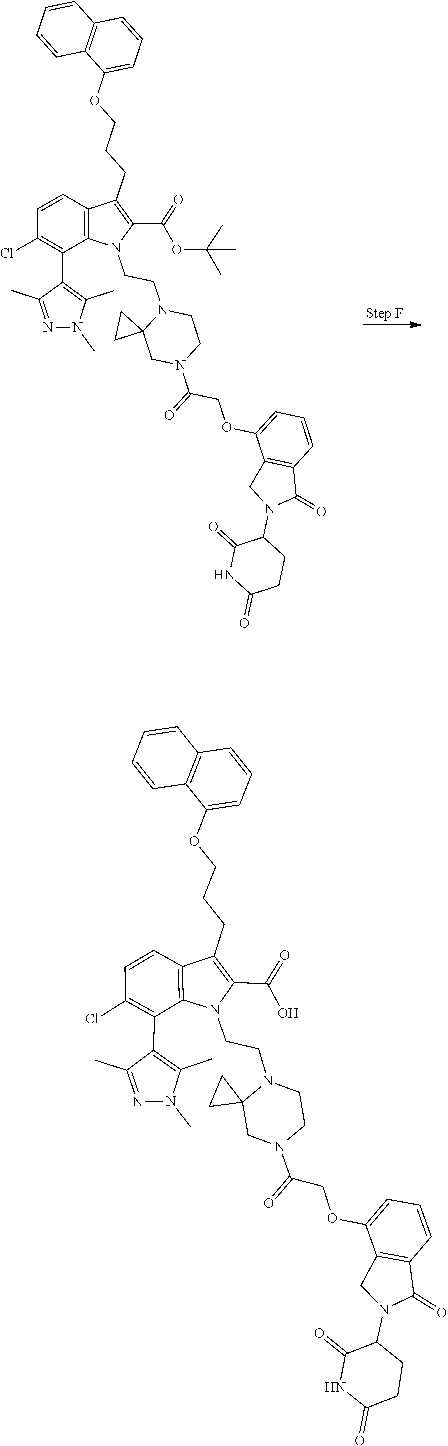 [SMILES: Cc1nn(C)c(C)c1-c1c(Cl)ccc2c(CCCOc3cccc4ccccc34)c(C(=O)OC(C)(C)C)n(CCN3CCN(C(=O)COc4cccc5c4CN(C4CCC(=O)NC4=O)C5=O)CC34CC4)c12 ; Cc1nn(C)c(C)c1-c1c(Cl)ccc2c(CCCOc3cccc4ccccc34)c(C(=O)O)n(CCN3CCN(C(=O)COc4cccc5c4CN(C4CCC(=O)NC4=O)C5=O)CC34CC4)c12]

To a solution of tert-butyl 6-chloro-1-{2-[7-(2-{[2-(2,6-dioxopiperidin-3-yl)-1-oxo-2,3-dihydro-1H-isoindol-4-yl]oxy}acetyl)-4,7-diazaspiro[2.5]octan-4-yl]ethyl}-3-[3-(naphthalen-1-yloxy)propyl]-7-(1,3,5-trimethyl-1H-pyrazol-4-yl)-1H-indole-2-carboxylate (85.0 mg, 0.087 mmol) in DCM (1.0 mL) was added TFA (1.0 mL, 13.059 mmol). The mixture was stirred for 18 h at RT. The crude was concentrated in vacuo and purified by preparative HPLC (C18, H₂O:MeCN+0.1% FA) to give 6-chloro-1-{2-[7-(2-{[2-(2,6-dioxopiperidin-3-yl)-1-oxo-2,3-dihydro-1H-isoindol-4-yl]oxy}acetyl)-4,7-diazaspiro[2.5]octan-4-yl]ethyl}-3-[3-(naphthalen-1-yloxy)propyl]-7-(1,3,5-trimethyl-1H-pyrazol-4-yl)-1H-indole-2-carboxylic acid (14.5 mg, 0.016 mmol, yield over 6 steps: 8.7%) as a white solid.

LCMS (ESI+): m/z 926.3 [M+H]⁺

¹H NMR (500 MHz, DMSO, 353K) δ 10.64 (s, 1H), 8.26-8.21 (m, 1H), 7.87-7.82 (m, 1H), 7.68-7.63 (m, 1H), 7.54-7.47 (m, 2H), 7.47-7.41 (m, 2H), 7.38 (t, J=7.9 Hz, 1H), 7.32 (d, J=7.5 Hz, 1H), 7.15 (dd, J=16.5, 8.4 Hz, 2H), 6.91 (d, J=7.5 Hz, 1H), 5.04 (dd, J=13.1, 5.2 Hz, 1H), 4.85 (bs, 2H), 4.40 (d, J=17.1 Hz, 1H), 4.32 (d, J=17.4 Hz, 1H), 4.25 (t, J=6.4 Hz, 2H), 4.12 (q, J=6.6 Hz, 2H), 3.76 (d, J=1.2 Hz, 3H), 3.36 (t, J=5.4 Hz, 2H), 3.27-3.21 (m, 2H), 3.12-3.04 (m, 2H), 2.93-2.83 (m, 1H), 2.62-2.57 (m, 1H), 2.48-2.38 (m, 1H), 2.24 (p, J=6.5 Hz, 2H), 2.09-1.98 (m, 5H), 1.91 (s, 3H), 0.29 (bs, 2H), 0.16 (bs, 2H).

4 protons in aliphatic area overlap with water.

### Example 64. 6-chloro-1-(2-(4-((2-(2,6-dioxopiperidin-3-yl)-1-oxoisoindolin-4-yl)carbamoyl)piperidin-1-yl)ethyl)-3-(3-(naphthalen-1-yloxy)propyl)-7-(1,3,5-trimethyl-1H-pyrazol-4-yl)-1H-indole-2-carboxylic acid (293)

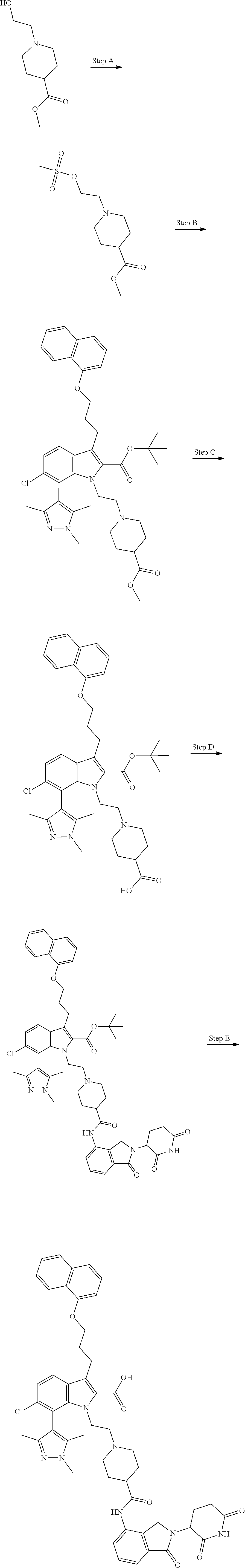

### Step A

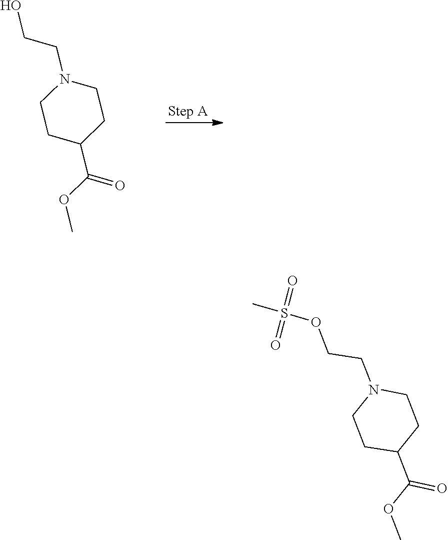 [SMILES: COC(=O)C1CCN(CCO)CC1 ; COC(=O)C1CCN(CCOS(C)(=O)=O)CC1]

Methyl 1-(2-hydroxyethyl)piperidine-4-carboxylate (584.5 mg, 3.122 mmol) was dissolved under argon atmosphere in dry DCM (31.2 mL) and Et₃N (0.651 mL, 4.683 mmol) along with DMAP (38.1 mg, 0.312 mmol) was added. Reaction mixture was cooled down to 0° C. and MsCl (0.290 mL, 3.746 mmol) was added dropwise. Reaction was allowed to stir in ambient temperature for 16 h. After that time TLC (SiO₂, 10% MeOH in DCM, visualization with ninhydrin) showed full conversion of the starting material. The solution was diluted with DCM (10 mL) and washed with brine and water. Organic layer was dried over anhydrous MgSO₄, filtered through a Schott funnel, concentrated and dried under reduced pressure to give crude methyl 1-[2-(methanesulfonyloxy)ethyl]piperidine-4-carboxylate (346.9 mg). The resulting product was used in the next step without further purification.

LCMS (ESI+): m/z 354.0 [M+H]⁺

### Step B

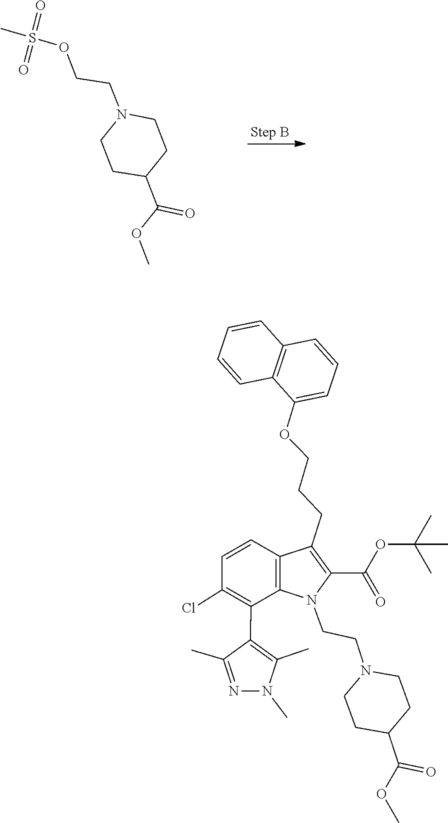 [SMILES: COC(=O)C1CCN(CCOS(C)(=O)=O)CC1 ; COC(=O)C1CCN(CCn2c(C(=O)OC(C)(C)C)c(CCCOc3cccc4ccccc34)c3ccc(Cl)c(-c4c(C)nn(C)c4C)c32)CC1]

tert-Butyl 6-chloro-3-[3-(naphthalen-1-yloxy)propyl]-7-(1,3,5-trimethyl-1H-pyrazol-4-yl)-1H-indole-2-carboxylate (50.0 mg, 0.092 mmol) was dissolved in DMF (2.2 mL) and Cs₂CO₃ (89.8 mg, 0.276 mmol) was added, followed by crude methyl 1-[2-(methanesulfonyloxy)ethyl]piperidine-4-carboxylate (53.6 mg, 0.202 mmol). Reaction (monitored with LCMS) was stirred in room temperature for 24 h in a sealed vial in room temperature. After complete conversion of the starting material the solution was diluted with DCM successfully washed with brine and water. Organic layer was dried over anhydrous MgSO₄, concentrated and dried under reduced pressure to give crude tert-butyl 6-chloro-1-{2-[4-(methoxycarbonyl)piperidin-1-yl]ethyl}-3-[3-(naphthalen-1-yloxy)propyl]-7-(1,3,5-trimethyl-1H-pyrazol-4-yl)-1H-indole-2-carboxylate (76.5 mg) as yellow oil.

LCMS (ESI+): m/z 713.90 [M+H]⁺

### Step C

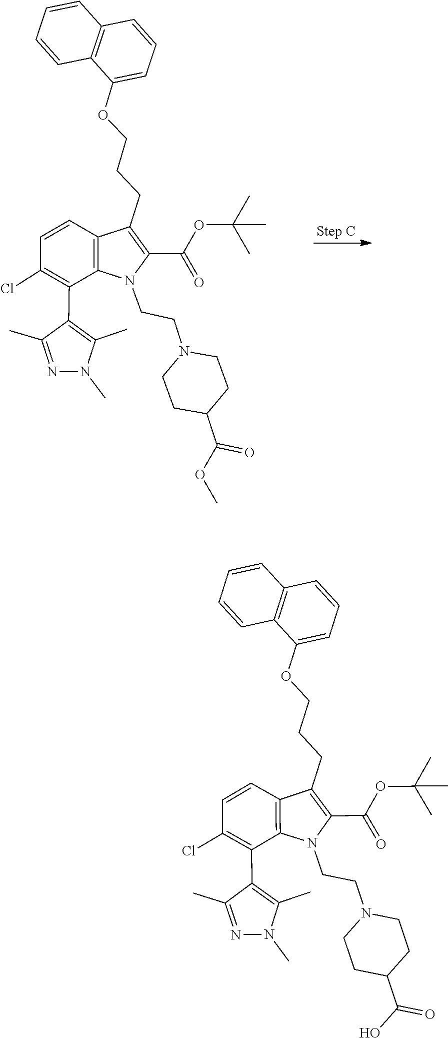 [SMILES: COC(=O)C1CCN(CCn2c(C(=O)OC(C)(C)C)c(CCCOc3cccc4ccccc34)c3ccc(Cl)c(-c4c(C)nn(C)c4C)c32)CC1 ; Cc1nn(C)c(C)c1-c1c(Cl)ccc2c(CCCOc3cccc4ccccc34)c(C(=O)OC(C)(C)C)n(CCN3CCC(C(=O)O)CC3)c12]

Crude tert-butyl 6-chloro-1-{2-[4-(methoxycarbonyl)piperidin-1-yl]ethyl}-3-[3-(naphthalen-1-yloxy)propyl]-7-(1,3,5-trimethyl-1H-pyrazol-4-yl)-1H-indole-2-carboxylate (76.5 mg, 0.107 mmol) was dissolved in THE (2.1 mL) and aqueous 1M LiOH (0.536 mL, 0.536 mmol) was added. Reaction, (monitored with LCMS), was stirred for 16 h in room temperature. After complete conversion of the starting material the solution pH was tuned to ˜6, resulting mixture was concentrated under reduced pressure and reaction product was partitioned between n-BuOH and water. Organic layer was dried over anhydrous MgSO₄, concentrated and dried under reduced pressure to get crude 1-(2-{2-[(tert-butoxy)carbonyl]-6-chloro-3-[3-(naphthalen-1-yloxy)propyl]-7-(1,3,5-trimethyl-1H-pyrazol-4-yl)-1H-indol-1-yl}ethyl)piperidine-4-carboxylic acid (50.0 mg, 0.072 mmol, 66.7%) which was used in the next step without further purification.

LCMS (ESI+): m/z 699.40 [M+H]⁺

### Step D

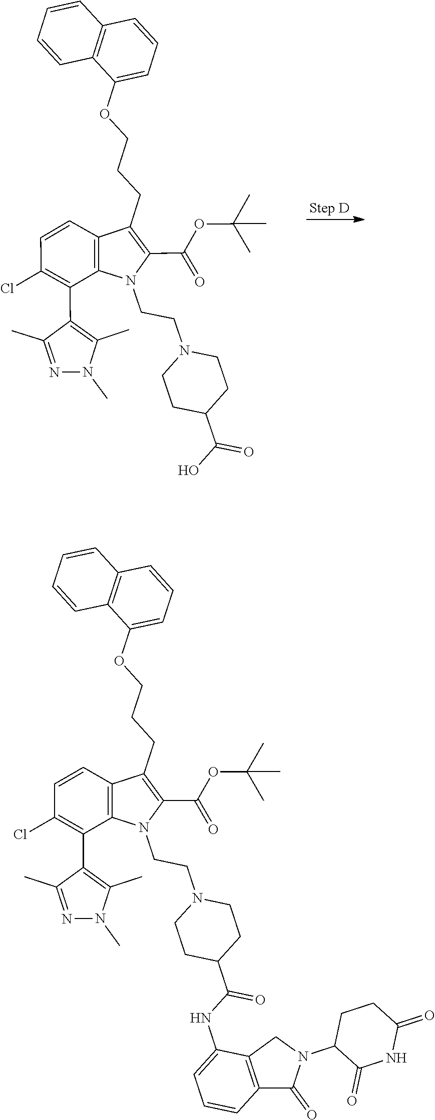

Crude 1-(2-{2-[(tert-butoxy)carbonyl]-6-chloro-3-[3-(naphthalen-1-yloxy)propyl]-7-(1,3,5-trimethyl-1H-pyrazol-4-yl)-1H-indol-1-yl}ethyl)piperidine-4-carboxylic acid (40.0 mg, 0.057 mmol), 3-(4-amino-1-oxo-2,3-dihydro-1H-isoindol-2-yl)piperidine-2,6-dione (16.3 mg, 0.063 mmol) and pyridine (0.009 mL, 0.114 mmol) were dissolved in dry DMF (1.1 mL), cooled down to 0° C. and T₃P (0.347 mL, 0.573 mmol, 50% in DMF) was added over next 6 days, maintaining the reaction in room temperature. DMF was evaporated and the resulting residue dissolved in dry THE (1.1 mL) and additional portions of T₃P (0.347 mL, 0.573 mmol, 50% in THF) were added over 24 h until no further reaction progress was observed (reaction monitored with LCMS). The mixture was concentrated and the resulting residue dissolved in DCM and washed with brine and water. Organic layer was dried over anhydrous MgSO₄, filtered, concentrated and dried under reduced pressure to give crude tert-butyl 6-chloro-1-[2-(4-{[2-(2,6-dioxopiperidin-3-yl)-1-oxo-2,3-dihydro-1H-isoindol-4-yl]carbamoyl}piperidin-1-yl)ethyl]-3-[3-(naphthalen-1-yloxy)propyl]-7-(1,3,5-trimethyl-1H-pyrazol-4-yl)-1H-indole-2-carboxylate as yellow oil, that was used directly in the next step.

LCMS (ESI+): m/z 940.35 [M+H]⁺

### Step E

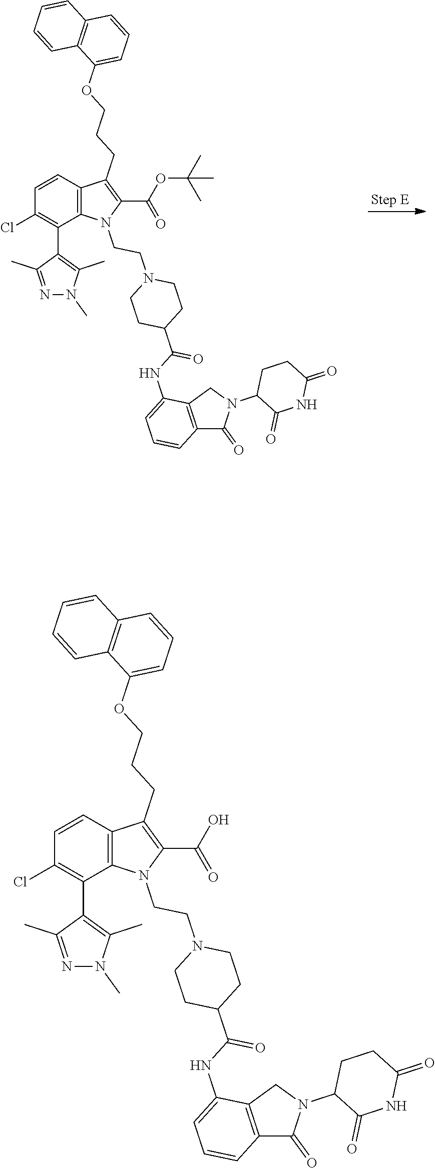 [SMILES: Cc1nn(C)c(C)c1-c1c(Cl)ccc2c(CCCOc3cccc4ccccc34)c(C(=O)OC(C)(C)C)n(CCN3CCC(C(=O)Nc4cccc5c4CN(C4CCC(=O)NC4=O)C5=O)CC3)c12 ; Cc1nn(C)c(C)c1-c1c(Cl)ccc2c(CCCOc3cccc4ccccc34)c(C(=O)O)n(CCN3CCC(C(=O)Nc4cccc5c4CN(C4CCC(=O)NC4=O)C5=O)CC3)c12]

Crude tert-butyl 6-chloro-1-[2-(4-{[2-(2,6-dioxopiperidin-3-yl)-1-oxo-2,3-dihydro-1H-isoindol-4-yl]carbamoyl}piperidin-1-yl)ethyl]-3-[3-(naphthalen-1-yloxy)propyl]-7-(1,3,5-trimethyl-1H-pyrazol-4-yl)-1H-indole-2-carboxylate (40.0 mg, 0.043 mmol) was dissolved in dry DCM (0.400 mL) under argon atmosphere and TFA (0.400 mL, 5.224 mmol) was added. Reaction was stirred in room temperature for 16 h. After complete consumption of the starting material (reaction monitored with LCMS), the mixture was concentrated with a strong stream of argon and dried under vacuum. The resulting residue was dissolved in DMSO, passed through a syringe filter and purified with preparative HPLC (H₂O:MeCN+0.1% FA) to give 6-chloro-1-[2-(4-{[2-(2,6-dioxopiperidin-3-yl)-1-oxo-2,3-dihydro-1H-isoindol-4-yl]carbamoyl}piperidin-1-yl)ethyl]-3-[3-(naphthalen-1-yloxy)propyl]-7-(1,3,5-trimethyl-1H-pyrazol-4-yl)-1H-indole-2-carboxylic acid (7.1 mg, 0.008 mmol, 8.69% yield over 4 steps) as white powder.

LCMS (ESI+): m/z 884.30 [M+H]⁺.

¹H NMR (500 MHz, DMSO, 353 K) δ 10.67 (s, 1H), 9.40 (s, 1H), 8.24-8.19 (m, 1H), 7.84 (dd, J=7.6, 1.7 Hz, 1H), 7.76 (dd, J=7.7, 1.2 Hz, 1H), 7.69 (d, J=8.5 Hz, 1H), 7.54-7.42 (m, 5H), 7.40-7.34 (m, 1H), 7.19 (d, J=8.5 Hz, 1H), 6.90 (dd, J=7.6, 1.1 Hz, 1H), 5.07 (dd, J=13.0, 5.2 Hz, 1H), 4.37 (s, 2H), 4.33-4.16 (m, 4H), 3.77 (s, 3H), 3.29-3.25 (m, 2H), 2.90-2.83 (m, 1H), 2.67-2.63 (m, 1H), 2.59-2.53 (m, 2H), 2.39 (dd, J=9.4, 4.5 Hz, 1H), 2.29-2.16 (m, 4H), 2.11-1.97 (m, 7H), 1.90 (s, 3H), 1.79-1.59 (m, 4H).

### Example 65: 294 and 295

### Example 65a: (Ra)-6-Chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (Intermediate-3A)

### Example 65b: (Sa)-6-Chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (Intermediate-3B)

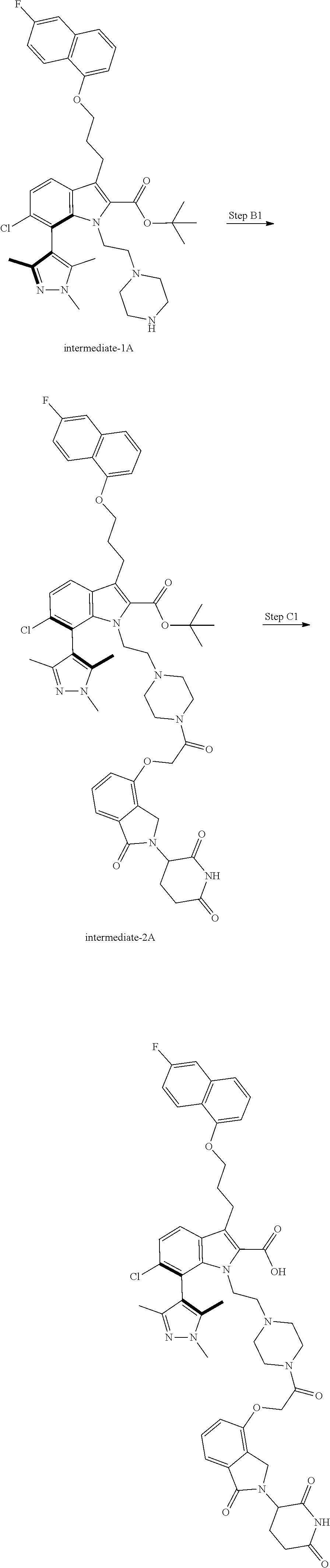

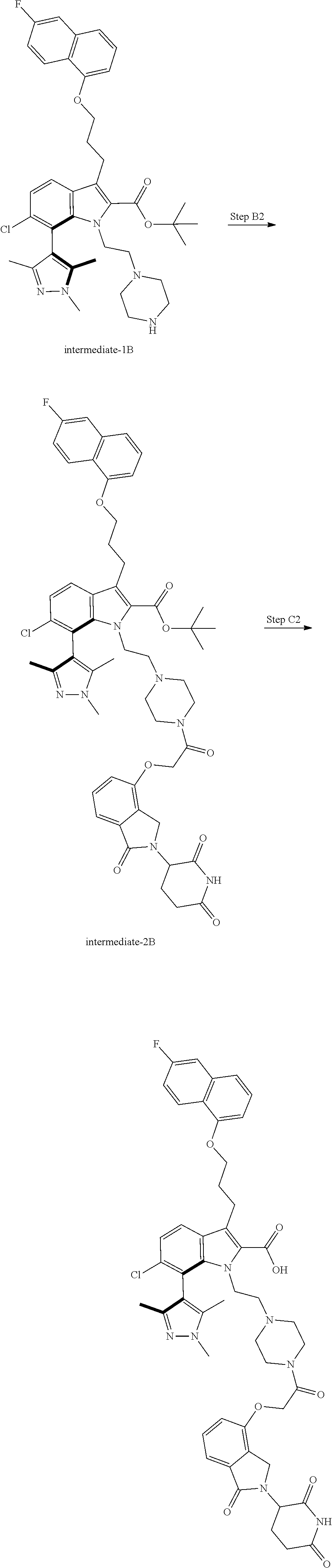

### Step A

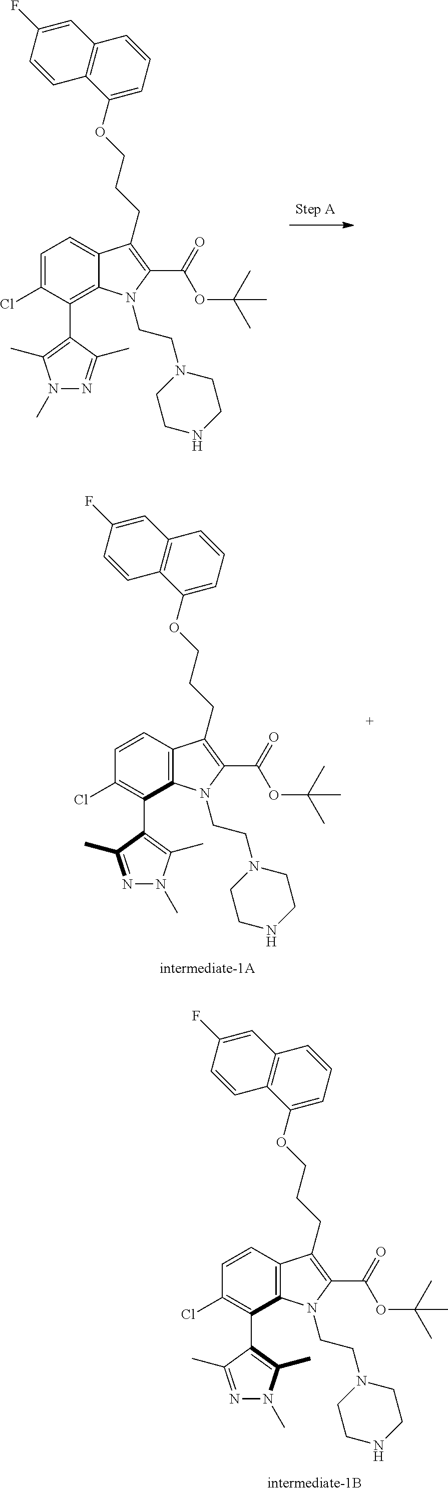

tert-Butyl 6-chloro-3-(3-((6-fluoronaphthalen-1-yl)oxy)propyl)-1-(2-(piperazin-1-yl)ethyl)-7-(1,3,5-trimethyl-1H-pyrazol-4-yl)-1H-indole-2-carboxylate (150 mg) was purified by chiral separation of afford of intermediate-1A (50 mg) as a white solid with 99.5% of LCMS purity and intermediate-1B (40 mg) as a white solid with 99.7% of LCMS purity. The two peaks were separately used to the next step. Absolute stereochemistry of compound intermediate-1A and intermediate-1B is arbitrarily assigned.

intermediate-1A LCMS (ESI+): m/z 674.49 [M+H]⁺

intermediate-1B LCMS (ESI+): m/z 674.49 [M+H]⁺

### Step B1

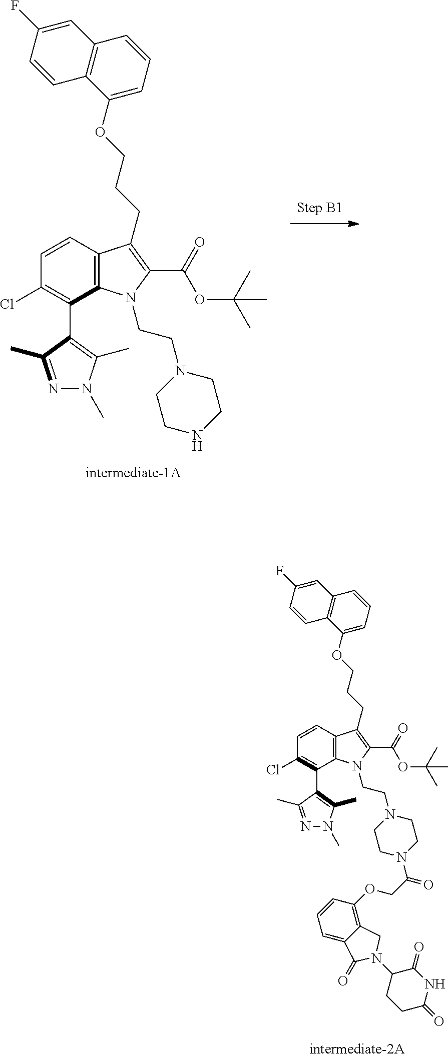

To a stirred suspension of 2-((2-(2,6-dioxopiperidin-3-yl)-1-oxoisoindolin-4-yl)oxy)acetic acid (17 mg, 0.053 mmol) in DMF (4 mL) was added HATU (33 mg, 0.089 mmol) and DIPEA (0.023 mL, 0.134 mmol) dropwise at 0° C. under nitrogen. The reaction mixture was then allowed to stir at ambient temperature for 15 minutes, after 15 min was added Intermediate-1A (30 mg, 0.045 mmol) in DMF (1 mL) to the reaction mixture and the mixture was stirred for 2 hours under nitrogen. After complete consumption of the starting material the reaction mixture was diluted with EtOAc, washed successively with water and brine, the organic layer was dried over Na₂SO₄ and evaporated under reduced pressure to get 40 mg crude intermediate-2A which was used next step without purification.

LCMS (ESI+): m/z 975.7 [M+H]⁺

### Step C1

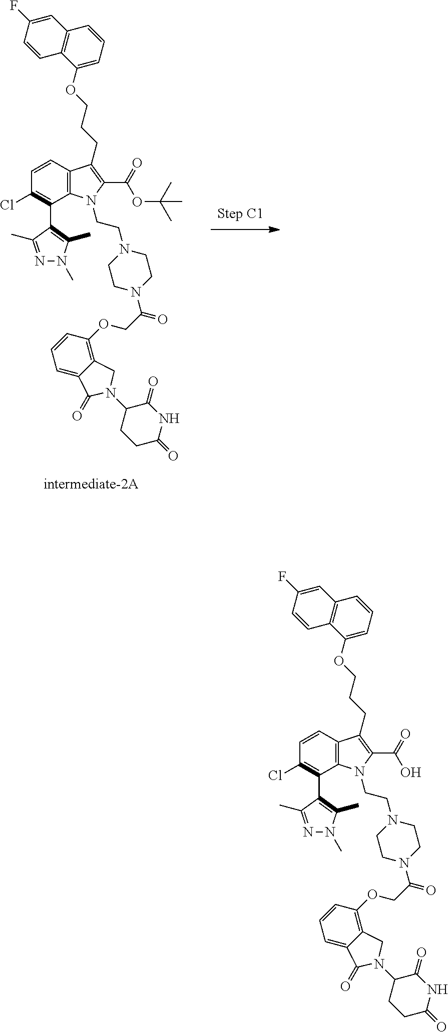

To a stirred suspension of intermediate-2A (40 mg, crude) in DCM (2 mL) was added TFA (2 mL) drop wise at 0° C. under nitrogen. The mixture was allowed to stir at room temperature for 16 h. After complete consumption of starting material, the volatiles were evaporated under reduced pressure to get the crude compound, which was then purified by reverse phase preparative HPLC (10 mM ammonium acetate in H₂O:MeCN) to get (Ra)-6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (16 mg, 0.017 mmol, 38% over two steps) as white solid.

LCMS (ESI+): m/z 918.6 [M+H]⁺

1H NMR (400 MHz, DMSO) δ=10.98 (s, 1H), 8.23 (dd, J=9.3, 5.8, 1H), 7.74 (d, J=8.6, 1H), 7.65 (dd, J=10.4, 2.7, 1H), 7.47-7.33 (m, 4H), 7.31 (d, J=7.4, 1H), 7.23 (d, J=8.5, 1H), 7.10 (d, J=8.2, 1H), 6.88 (dd, J=5.1, 3.5, 1H), 5.10 (dd, J=13.3, 5.1, 1H), 4.95 (s, 2H), 4.37 (d, J=17.4, 1H), 4.32-4.09 (m, 5H), 3.75 (d, J=1.5, 3H), 2.99-2.84 (m, 1H), 2.63-2.53 (m, 1H), 2.47-2.37 (m, 1H), 2.20 (p, J=6.7, 2H), 2.15-1.90 (m, 11H), 1.87 (s, 3H).

### Step B2

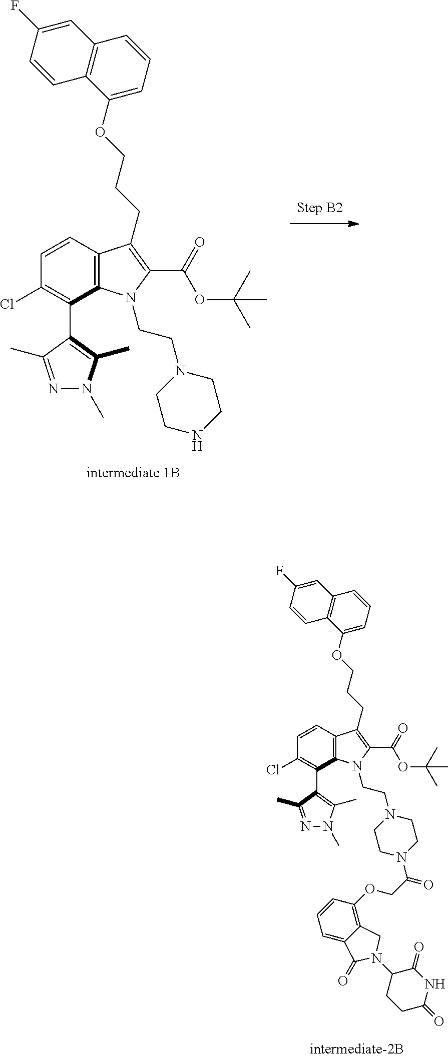

To a stirred suspension of 2-((2-(2,6-dioxopiperidin-3-yl)-1-oxoisoindolin-4-yl)oxy)acetic acid (17 mg, 0.053 mmol) in DMF (4 mL) was added HATU (33 mg, 0.089 mmol) and DIPEA (0.023 mL, 0.134 mmol) dropwise at 0° C. under nitrogen. The reaction mixture was then allowed to stir at ambient temperature for 15 minutes, after 15 min was added Intermediate-1B (30 mg, 0.045 mmol) in DMF (1 mL) to the reaction mixture and the mixture was stirred for 2 hours under nitrogen. After complete consumption of the starting material the reaction mixture was diluted with EtOAc, washed successively with water and brine, the organic layer was dried over Na₂SO₄ and evaporated under reduced pressure to get 40 mg crude intermediate-2B which was used next step without purification.

LCMS (ESI+): m/z 975.7 [M+H]⁺

### Step C2

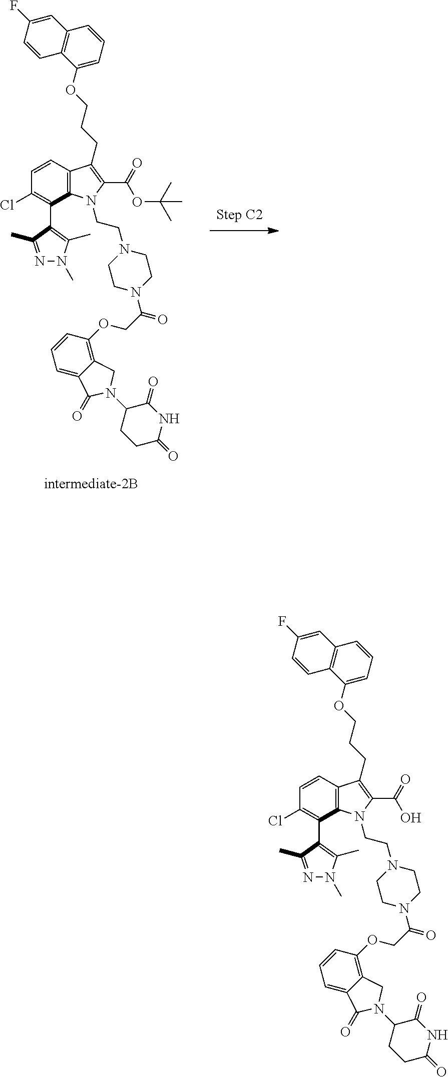

To a stirred suspension of intermediate-2B (40 mg, crude) in DCM (2 mL) was added TFA (2 mL) drop wise at 0° C. under nitrogen. The mixture was allowed to stir at room temperature for 16 h. After complete consumption of SM the volatiles were evaporated under reduced pressure to get the crude compound, which was then purified by reverse phase preparative HPLC (10 mM ammonium acetate in H₂O:MeCN) to get (Sa)-6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (13 mg, 0.014 mmol, 31% over two steps) as white solid.

LCMS (ESI+): m/z 918.6 [M+H]⁺

¹H NMR (400 MHz, DMSO) δ 10.98 (s, 1H), 8.23 (dd, J=9.2, 5.9 Hz, 1H), 7.74 (d, J=8.6 Hz, 1H), 7.65 (dd, J=10.4, 2.7 Hz, 1H), 7.47-7.39 (m, 3H), 7.36 (td, J=8.9, 2.7 Hz, 1H), 7.31 (d, J=7.5 Hz, 1H), 7.23 (d, J=8.5 Hz, 1H), 7.10 (d, J=8.2 Hz, 1H), 6.92-6.80 (m, 1H), 5.10 (dd, J=13.3, 5.0 Hz, 1H), 4.95 (s, 2H), 4.37 (d, J=17.4 Hz, 1H), 4.32-4.07 (m, 5H), 3.75 (d, 3H), 3.31-3.19 (m, 6H), 2.97-2.85 (m, 1H), 2.63-2.53 (m, 1H), 2.46-2.36 (m, 1H), 2.20 (p, J=7.4 Hz, 2H), 2.15-1.89 (m, 10H), 1.87 (s, 3H).

### Example 66: 296 and 297

### (S)-6-Chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (296)

### (R)-6-Chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (297)

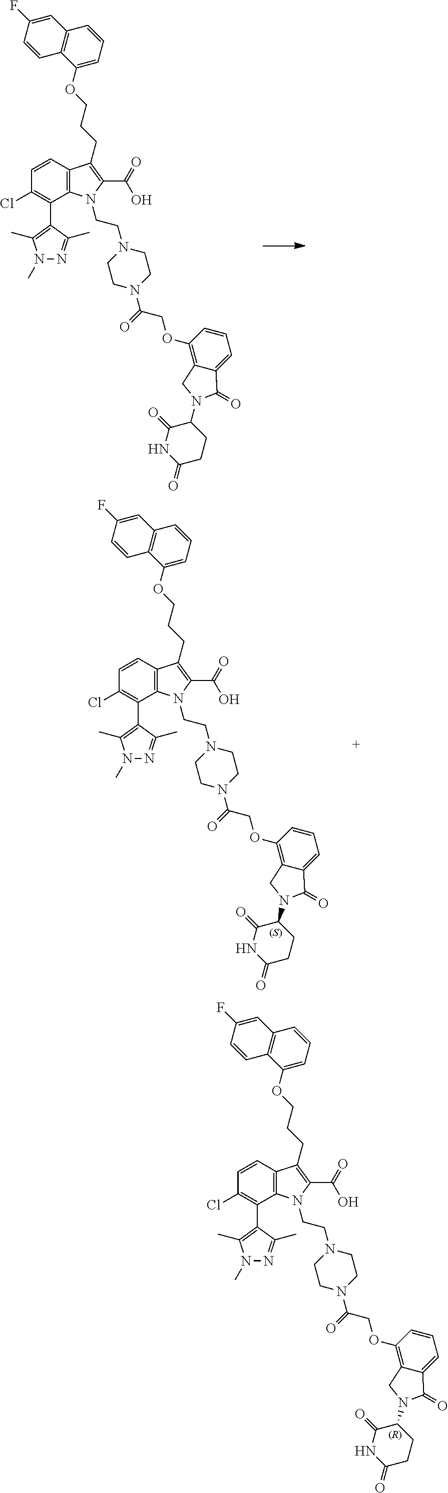

6-Chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (450 mg, 0.49 mmol)) was purified by chiral separation (Chiralpack IC, 0.1% TFA in the mixture of 20% hexane, 40% DCM and 40% isopropanol) to afford: (S)-6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (160 mg, 0.17 mmol) as white solid.

LCMS (ESI+): m/z 918.5 [M+H]⁺

¹H NMR (500 MHz, DMSO, 353 K) δ=10.66 (s, 1H), 8.26 (dd, J=9.2, 5.9, 1H), 7.76 (d, J=8.6, 1H), 7.61 (dd, J=10.4, 2.6, 1H), 7.50-7.40 (m, 3H), 7.38-7.30 (m, 2H), 7.26 (d, J=8.5, 1H), 7.18 (d, J=8.2, 0.7, 1H), 6.90 (dd, J=5.7, 2.9, 1H), 5.07 (dd, J=13.0, 5.2, 1H), 4.94 (s, 2H), 4.43 (d, J=17.2, 1H), 4.39-4.25 (m, 5H), 3.78 (d, J=2.1, 3H), 3.50-3.47 (m, 4H), 3.34-3.31 (m, 2H), 2.90 (ddd, J=17.4, 13.4, 5.5, 1H), 2.68-2.62 (m, 1H), 2.55-2.53 (m, 1H), 2.46-2.34 (m, 6H), 2.29-2.22 (m, 2H), 2.11-2.02 (m, 4H), 1.92 (s, 3H).

and (R)-6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (165 mg, 0.18 mmol) as a white solid

LCMS (ESI+): m/z 918.5 [M+H]⁺

¹H NMR (500 MHz, DMSO, 353 K) δ 10.65 (s, 1H), 8.26 (dd, J=9.2, 5.9 Hz, 1H), 7.75 (d, J=8.6 Hz, 1H), 7.60 (dd, J=10.4, 2.6 Hz, 1H), 7.48-7.42 (m, 3H), 7.38-7.31 (m, 2H), 7.25 (d, J=8.6 Hz, 1H), 7.17 (d, J=8.1 Hz, 1H), 6.89 (dd, J=5.7, 2.9 Hz, 1H), 5.07 (dd, J=13.0, 5.2 Hz, 1H), 4.93 (s, 2H), 4.42 (d, J=17.1 Hz, 1H), 4.37-4.22 (m, 5H), 3.78 (d, J=1.9 Hz, 3H), 3.46-3.40 (m, 4H), 3.34-3.29 (m, 2H), 2.90 (ddd, J=17.3, 13.4, 5.5 Hz, 1H), 2.69-2.62 (m, 1H), 2.55-2.53 (m, 1H), 2.48-2.42 (m, 1H), 2.32-2.22 (m, 7H), 2.11-2.00 (m, 4H), 1.91 (s, 3H).

Absolute stereochemistry of compounds is arbitrarily assigned.

### Example 67. 1-(2-(2-Carboxy-6-chloro-3-(3-((6-fluoronaphthalen-1-yl)oxy)propyl)-7-(1,3,5-trimethyl-1H-pyrazol-4-yl)-1H-indol-1-yl)ethyl)-4-(2-((2-(2,6-dioxopiperidin-3-yl)-1-oxoisoindolin-4-yl)oxy)acetyl)piperazine 1-oxide (298)

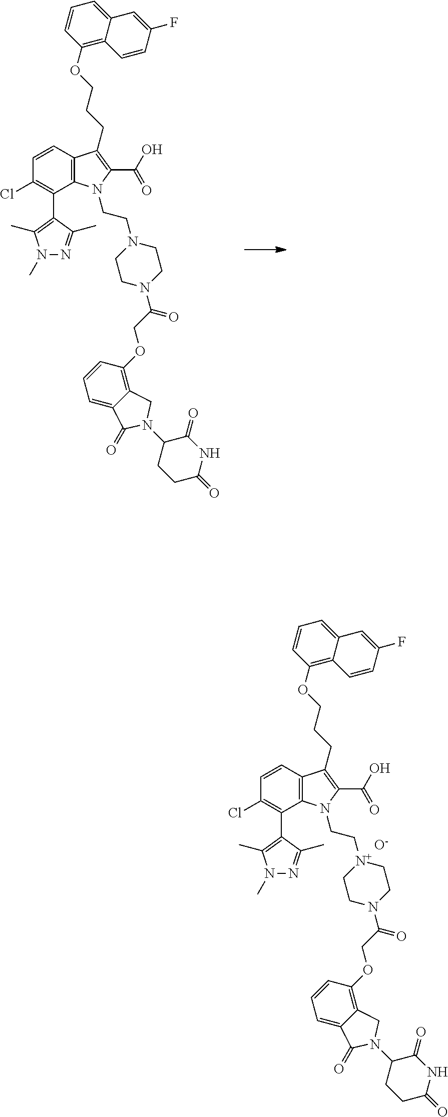

6-Chloro-1-{2-[4-(2-{[2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (50.0 mg, 0.054 mmol) was dissolved in EtOAc (2.0 mL) and m-CPBA (13.4 mg, 0.060 mmol) as EtOAc solution (0.7 mL) was added. The reaction was stirred in room temperature and monitored with LCMS. After 15 min complete conversion was noted. The solution was filtered through silica pad and washed with EtOAc. Reaction product was removed from silica with MeOH and the solution was concentrated under reduced pressure. The resulting residue was dissolved in DMSO, passed through syringe filter and purified with preparative HPLC (H₂O:MeCN+0.1% FA) to give 1-(2-(2-carboxy-6-chloro-3-(3-((6-fluoronaphthalen-1-yl)oxy)propyl)-7-(1,3,5-trimethyl-1H-pyrazol-4-yl)-1H-indol-1-yl)ethyl)-4-(2-((2-(2,6-dioxopiperidin-3-yl)-1-oxoisoindolin-4-yl)oxy)acetyl)piperazine 1-oxide (22.6 mg, 0.024 mmol, 44.4%) as white solid.

LCMS (ESI+): m/z 934.25 [M+H]⁺

¹H NMR (500 MHz, DMSO) δ 13.03 (s, 1H), 10.98 (s, 1H), 8.27 (dd, J=9.3, 5.9 Hz, 1H), 7.68-7.62 (m, 2H), 7.46 (d, J=7.8 Hz, 1H), 7.44-7.42 (m, 2H), 7.38 (td, J=8.9, 2.7 Hz, 1H), 7.33 (d, J=7.4 Hz, 1H), 7.23 (d, J=8.5 Hz, 1H), 7.17 (d, J=8.2 Hz, 1H), 6.88 (p, J=4.7 Hz, 1H), 5.20-4.97 (m, 3H), 4.70-4.44 (m, 1H), 4.43-4.18 (m, 4H), 4.15 (t, J=6.5 Hz, 2H), 4.01-3.82 (m, 1H), 3.82-3.74 (m, 3H), 3.62 (s, 5H), 3.15-3.06 (m, 4H), 2.92 (ddd, J=17.2, 13.5, 5.4 Hz, 1H), 2.62-2.54 (m, 1H), 2.47-2.40 (m, 1H), 2.24-2.12 (m, 2H), 2.12-1.93 (m, 5H), 1.92-1.79 (m, 3H).

### Example 68. 6-Chloro-1-(2-(1-(2-((2-(2,6-dioxopiperidin-3-yl)-1-oxoisoindolin-4-yl)oxy)acetyl)azetidin-3-yl)ethyl)-3-(3-((6-fluoronaphthalen-1-yl)oxy)propyl)-7-(1,3,5-trimethyl-1H-pyrazol-4-yl)-1H-indole-2-carboxylic acid (299)

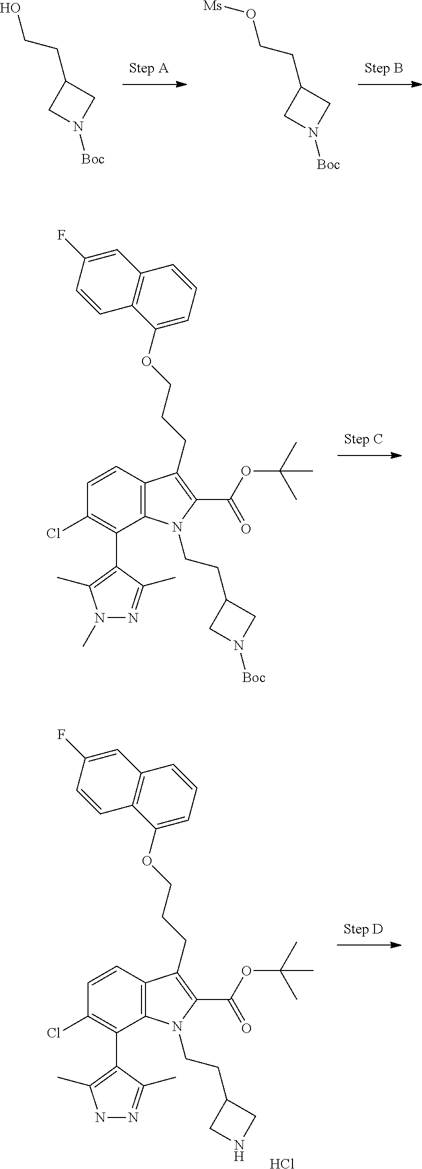

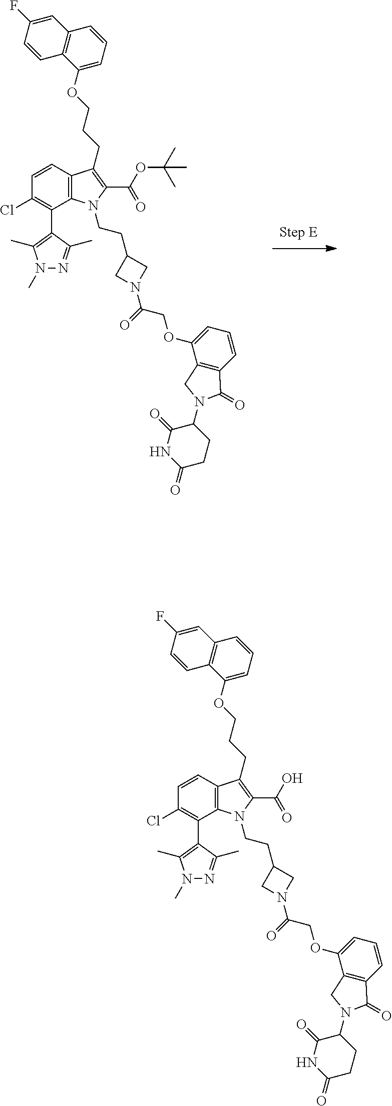 [SMILES: Cc1nn(C)c(C)c1-c1c(Cl)ccc2c(CCCOc3cccc4cc(F)ccc34)c(C(=O)OC(C)(C)C)n(CCC3CN(C(=O)COc4cccc5c4CN(C4CCC(=O)NC4=O)C5=O)C3)c12 ; Cc1nn(C)c(C)c1-c1c(Cl)ccc2c(CCCOc3cccc4cc(F)ccc34)c(C(=O)O)n(CCC3CN(C(=O)COc4cccc5c4CN(C4CCC(=O)NC4=O)C5=O)C3)c12]

### Step A

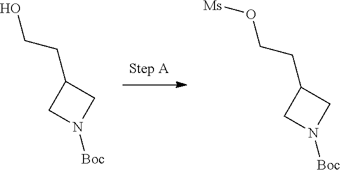 [SMILES: CC(C)(C)OC(=O)N1CC(CCO)C1 ; CC(C)(C)OC(=O)N1CC(CCOS(C)(=O)=O)C1]

tert-Butyl 3-(2-hydroxyethyl)azetidine-1-carboxylate (100.0 mg, 0.497 mmol) was dissolved in DCM (5.0 mL), Et₃N (0.104 mL, 0.745 mmol) and DMAP (6.1 mg, 0.050 mmol) were added and reaction mixture cooled to 0° C. Then MsCl (0.046 mL, 0.596 mmol) was added dropwise and reaction mixture was let to stir at room temperature for 1 h. After completed consumption of staring material (monitored by TLC, 5% MeOH in DCM), the crude was washed with brine, dried over Na₂SO₄, filtered and concentrated in vacuo. The isolated product tert-butyl 3-[2-(methanesulfonyloxy)ethyl]azetidine-1-carboxylate (137.9 mg, crude) was a yellow oil.

### Step B

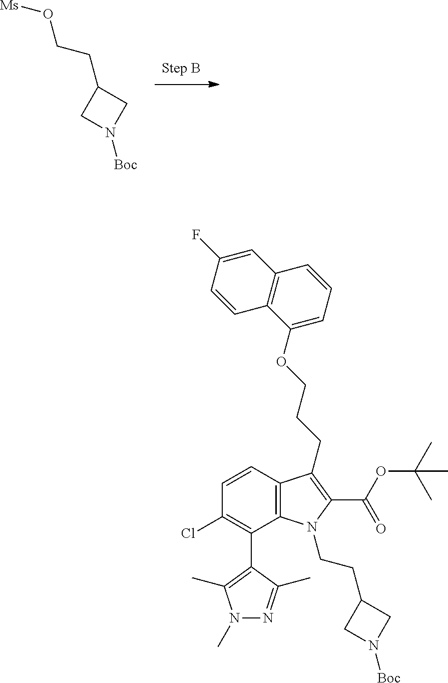 [SMILES: CC(C)(C)OC(=O)N1CC(CCOS(C)(=O)=O)C1 ; Cc1nn(C)c(C)c1-c1c(Cl)ccc2c(CCCOc3cccc4cc(F)ccc34)c(C(=O)OC(C)(C)C)n(CCC3CN(C(=O)OC(C)(C)C)C3)c12]

tert-Butyl 6-chloro-3-{3-[(6-fluoronaphthalen-1-yl)oxy]propyl}-7-(1,3,5-trimethyl-1H-pyrazol-4-yl)-1H-indole-2-carboxylate (30.0 mg, 0.053 mmol), tert-butyl 3-[2-(methanesulfonyloxy)ethyl]azetidine-1-carboxylate (22.4 mg, 0.080 mmol) and Cs₂CO₃ (52.2 mg, 0.160 mmol) were dissolved in dry DMF (2.0 mL) in an inert atmosphere and stirred at 60° C. for 18 h. After complete consumption of the starting material (monitored by LCMS), the residues were dissolved in DCM and washed with brine. The organic layer was dried over Na₂SO₄, filtered and concentrated in vacuo to give 32.3 mg of crude product tert-butyl 1-(2-{1-[(tert-butoxy)carbonyl]azetidin-3-yl}ethyl)-6-chloro-3-{3-[(6-fluoronaphthalen-1-yl)oxy]propyl}-7-(1,3,5-trimethyl-1H-pyrazol-4-yl)-1H-indole-2-carboxylate which was used to next step without further purification.

LCMS (ESI+): m/z 745.7 [M+H]⁺

### Step C

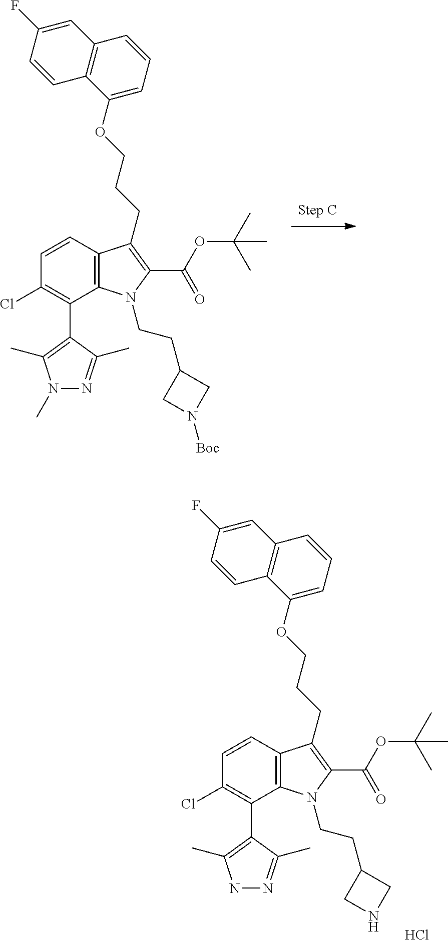 [SMILES: Cc1nn(C)c(C)c1-c1c(Cl)ccc2c(CCCOc3cccc4cc(F)ccc34)c(C(=O)OC(C)(C)C)n(CCC3CN(C(=O)OC(C)(C)C)C3)c12 ; Cc1n[nH]c(C)c1-c1c(Cl)ccc2c(CCCOc3cccc4cc(F)ccc34)c(C(=O)OC(C)(C)C)n(CCC3CNC3)c12 ; Cl]

tert-Butyl 1-(2-{1-[(tert-butoxy)carbonyl]azetidin-3-yl}ethyl)-6-chloro-3-{3-[(6-fluoronaphthalen-1-yl)oxy]propyl}-7-(1,3,5-trimethyl-1H-pyrazol-4-yl)-1H-indole-2-carboxylate (32.3 mg crude) was suspended in dry THE (2 mL), cooled to 0° C. and to the mixture was added 4M HCl in dioxane (1 mL, 28.799 mmol). The reaction was stirred at room temperature for 18 h. The crude was concentrated in vacuo to get 30 mg crude of tert-butyl 1-[2-(azetidin-3-yl)ethyl]-6-chloro-3-{3-[(6-fluoronaphthalen-1-yl)oxy]propyl}-7-(1,3,5-trimethyl-1H-pyrazol-4-yl)-1H-indole-2-carboxylate hydrochloride as a yellowish solid, which was used to next step without further purification.

### Step D

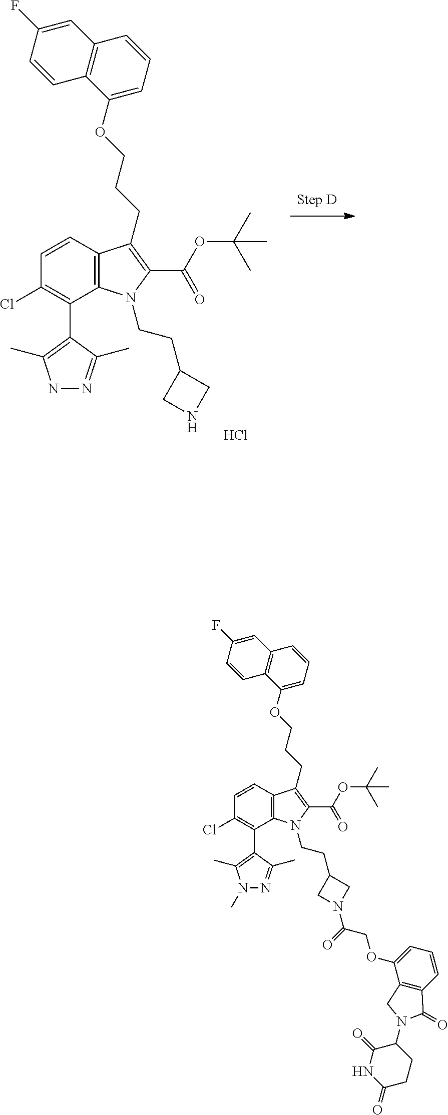 [SMILES: Cc1n[nH]c(C)c1-c1c(Cl)ccc2c(CCCOc3cccc4cc(F)ccc34)c(C(=O)OC(C)(C)C)n(CCC3CNC3)c12 ; Cl ; Cc1nn(C)c(C)c1-c1c(Cl)ccc2c(CCCOc3cccc4cc(F)ccc34)c(C(=O)OC(C)(C)C)n(CCC3CN(C(=O)COc4cccc5c4CN(C4CCC(=O)NC4=O)C5=O)C3)c12]

To a solution of tert-butyl 1-[2-(azetidin-3-yl)ethyl]-6-chloro-3-{3-[(6-fluoronaphthalen-1-yl)oxy]propyl}-7-(1,3,5-trimethyl-1H-pyrazol-4-yl)-1H-indole-2-carboxylate hydrochloride (20 mg, crude), 2-{[2-(2,6-dioxopiperidin-3-yl)-1-oxo-2,3-dihydro-1H-isoindol-4-yl]oxy}acetic acid (11.2 mg, 0.035 mmol) and HATU (22.3 mg, 0.059 mmol) in dry DMF (2.0 mL) was added DIPEA (0.015 mL, 0.088 mmol). The mixture was stirred at room temperature for 30 min. The crude was diluted with DCM and washed with brine. The organic layer was dried over MgSO₄, filtered and concentrated in vacuo to get 41 mg crude with tert-butyl 6-chloro-1-{2-[1-(2-{[2-(2,6-dioxopiperidin-3-yl)-1-oxo-2,3-dihydro-1H-isoindol-4-yl]oxy}acetyl)azetidin-3-yl]ethyl}-3-{3-[(6-fluoronaphthalen-1-yl)oxy]propyl}-7-(1,3,5-trimethyl-1H-pyrazol-4-yl)-1H-indole-2-carboxylate as yellow oil which was used to next step without further purification.

LCMS (ESI+): m/z 945.35 [M+H]⁺

### Step E

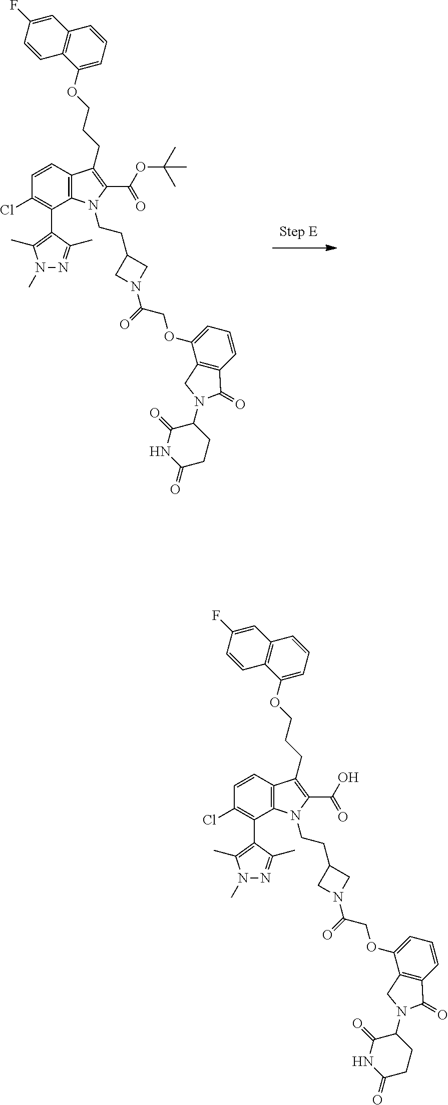 [SMILES: Cc1nn(C)c(C)c1-c1c(Cl)ccc2c(CCCOc3cccc4cc(F)ccc34)c(C(=O)OC(C)(C)C)n(CCC3CN(C(=O)COc4cccc5c4CN(C4CCC(=O)NC4=O)C5=O)C3)c12 ; Cc1nn(C)c(C)c1-c1c(Cl)ccc2c(CCCOc3cccc4cc(F)ccc34)c(C(=O)O)n(CCC3CN(C(=O)COc4cccc5c4CN(C4CCC(=O)NC4=O)C5=O)C3)c12]

To a solution of tert-butyl 6-chloro-1-{2-[1-(2-{[2-(2,6-dioxopiperidin-3-yl)-1-oxo-2,3-dihydro-1H-isoindol-4-yl]oxy}acetyl)azetidin-3-yl]ethyl}-3-{3-[(6-fluoronaphthalen-1-yl)oxy]propyl}-7-(1,3,5-trimethyl-1H-pyrazol-4-yl)-1H-indole-2-carboxylate (41 mg, crude) in DCM (1.0 mL) was added TFA (1.0 mL, 13.059 mmol). The mixture was stirred for 18 h at RT. The crude was concentrated in vacuo and purified by preparative HPLC (C18, H₂O:MeCN+0.1% FA) to obtain 6-chloro-1-{2-[1-(2-{[2-(2,6-dioxopiperidin-3-yl)-1-oxo-2,3-dihydro-1H-isoindol-4-yl]oxy}acetyl)azetidin-3-yl]ethyl}-3-{3-[(6-fluoronaphthalen-1-yl)oxy]propyl}-7-(1,3,5-trimethyl-1H-pyrazol-4-yl)-1H-indole-2-carboxylic acid (1.0 mg, 0.001 mmol, yield over 5 steps 3%) as a white solid.

LCMS (ESI+): m/z 889.25 [M+H]⁺

¹H NMR (500 MHz, DMSO) δ 10.64 (s, 1H), 8.24 (dd, J=9.3, 5.9 Hz, 1H), 7.72-7.68 (m, 1H), 7.58 (dd, J=10.3, 2.6 Hz, 1H), 7.46 (t, J=7.7 Hz, 1H), 7.43-7.41 (m, 2H), 7.35-7.30 (m, 2H), 7.20 (d, J=8.9 Hz, 1H), 7.15 (d, J=8.1 Hz, 1H), 6.88 (dd, J=5.5, 3.3 Hz, 1H), 5.05 (dd, J=13.3, 5.3 Hz, 1H), 4.66 (s, 2H), 4.45-4.31 (m, 2H), 4.24 (t, J=6.2 Hz, 2H), 4.19-4.10 (m, 1H), 4.10-4.02 (m, 1H), 3.75 (d, J=4.8 Hz, 3H), 3.28 (t, J=7.6 Hz, 2H), 2.93-2.84 (m, 1H), 2.63-2.57 (m, 2H), 2.26-2.20 (m, 2H), 2.07-1.98 (m, 9H), 1.88 (s, 3H), 1.52-1.48 (m, 2H).

### Example 69. 6-Chloro-1-(2-(5-((2-(2,6-dioxopiperidin-3-yl)-1,3-dioxoisoindolin-4-yl)glycyl)hexahydropyrrolo[3,4-b]pyrrol-1(2H)-yl)ethyl)-3-(3-((6-fluoronaphthalen-1-yl)oxy)propyl)-7-(1,3,5-trimethyl-1H-pyrazol-4-yl)-1H-indole-2-carboxylic acid (300)

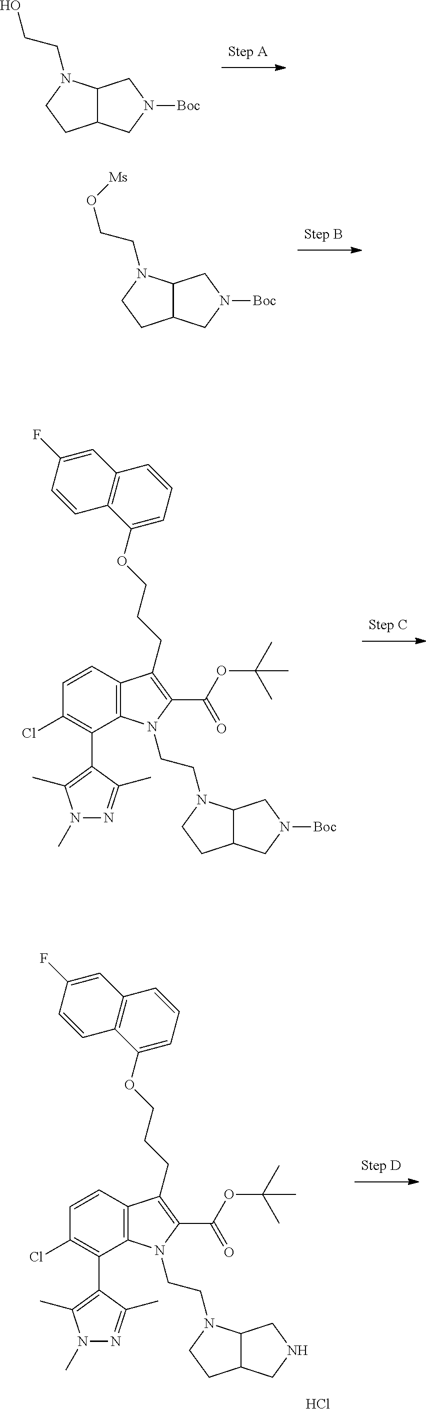

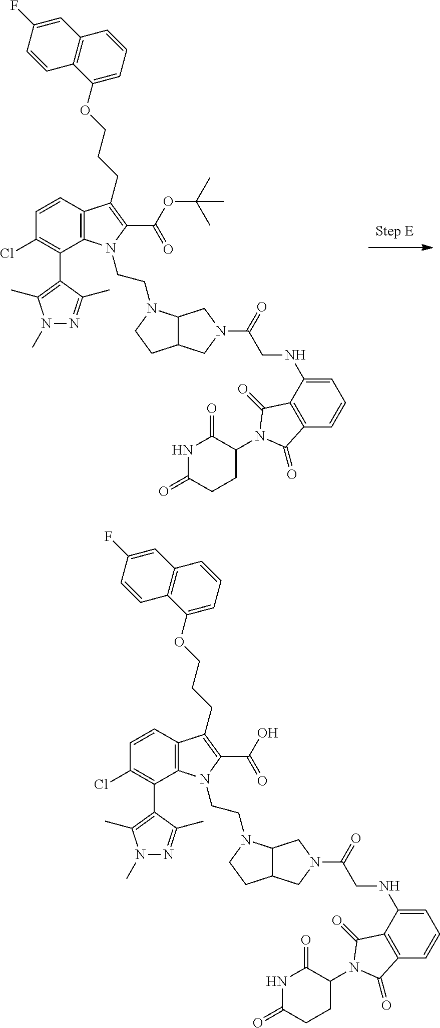 [SMILES: Cc1nn(C)c(C)c1-c1c(Cl)ccc2c(CCCOc3cccc4cc(F)ccc34)c(C(=O)OC(C)(C)C)n(CCN3CCC4CN(C(=O)CNc5cccc6c5C(=O)N(C5CCC(=O)NC5=O)C6=O)CC43)c12 ; Cc1nn(C)c(C)c1-c1c(Cl)ccc2c(CCCOc3cccc4cc(F)ccc34)c(C(=O)O)n(CCN3CCC4CN(C(=O)CNc5cccc6c5C(=O)N(C5CCC(=O)NC5=O)C6=O)CC43)c12]

### Step A

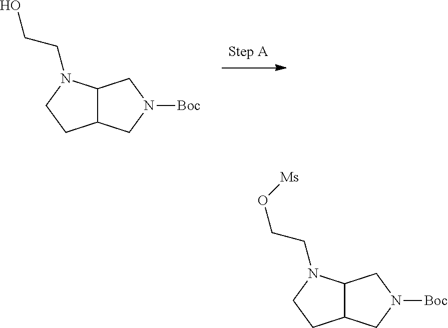 [SMILES: CC(C)(C)OC(=O)N1CC2CCN(CCO)C2C1 ; CC(C)(C)OC(=O)N1CC2CCN(CCOS(C)(=O)=O)C2C1]

tert-Butyl 1-(2-hydroxyethyl)-octahydropyrrolo[2,3-c]pyrrole-5-carboxylate (332.0 mg, 1.295 mmol) was dissolved in DCM (5.0 mL) and Et₃N (0.270 mL, 1.943 mmol) was added to the mixture and reaction was cooled to 0° C. Then MsCl (0.150 mL, 1.943 mmol) was added dropwise and reaction mixture was let to stir at room temperature for 18 h. The crude was extracted with brine, dried over Na₂SO₄, filtered and concentrated in vacuo. The presence of product was checked by TLC (5% MeOH in DCM). tert-Butyl 1-[2-(methanesulfonyloxy)ethyl]-octahydropyrrolo[2,3-c]pyrrole-5-carboxylate (292.0 mg, crude) was an orange solid and was used to next step without further purification.

### Step B

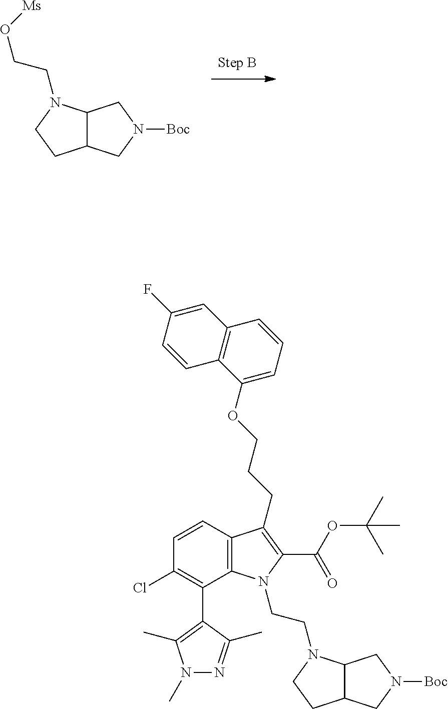 [SMILES: CC(C)(C)OC(=O)N1CC2CCN(CCOS(C)(=O)=O)C2C1 ; Cc1nn(C)c(C)c1-c1c(Cl)ccc2c(CCCOc3cccc4cc(F)ccc34)c(C(=O)OC(C)(C)C)n(CCN3CCC4CN(C(=O)OC(C)(C)C)CC43)c12]

tert-Butyl 6-chloro-3-{3-[(6-fluoronaphthalen-1-yl)oxy]propyl}-7-(1,3,5-trimethyl-1H-pyrazol-4-yl)-1H-indole-2-carboxylate (60.0 mg, 0.107 mmol), tert-butyl 1-[2-(methanesulfonyloxy)ethyl]-octahydropyrrolo[2,3-c]pyrrole-5-carboxylate (53.5 mg, crude) and Cs₂CO₃ (104.3 mg, 0.320 mmol) were dissolved in dry DMF (2.0 mL) in an inert atmosphere and was stirred and stirred at 60° C. for 18 h. After complete consumption of the starting material (monitored by LCMS), the residues were dissolved in DCM and washed with brine. The organic layer was dried over Na₂SO₄, filtered and concentrated in vacuo to give a 175 mg of crude tert-butyl 1-(2-{5-[(tert-butoxy)carbonyl]-octahydropyrrolo[2,3-c]pyrrol-1-yl}ethyl)-6-chloro-3-{3-[(6-fluoronaphthalen-1-yl)oxy]propyl}-7-(1,3,5-trimethyl-1H-pyrazol-4-yl)-3a,7a-dihydro-1H-indole-2-carboxylate which was used to next step without further purification.

LCMS (ESI+): m/z 802.1 [M+H]⁺

### Step C

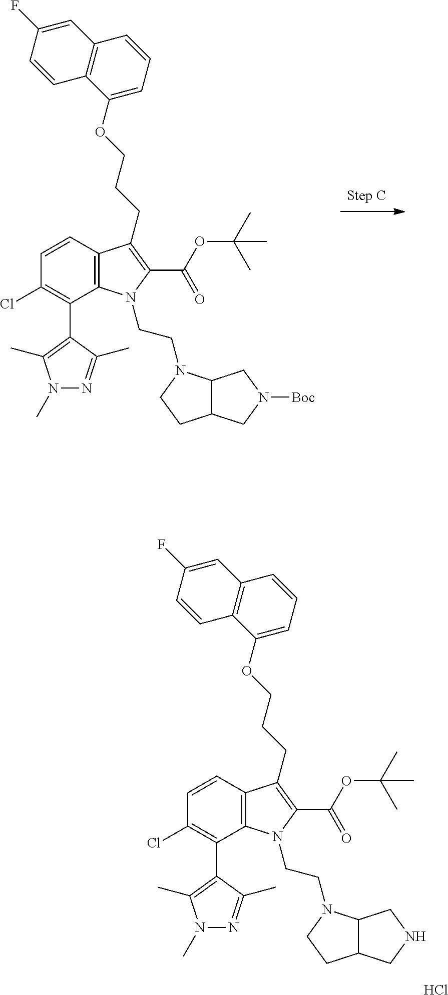 [SMILES: Cc1nn(C)c(C)c1-c1c(Cl)ccc2c(CCCOc3cccc4cc(F)ccc34)c(C(=O)OC(C)(C)C)n(CCN3CCC4CN(C(=O)OC(C)(C)C)CC43)c12 ; Cc1nn(C)c(C)c1-c1c(Cl)ccc2c(CCCOc3cccc4cc(F)ccc34)c(C(=O)OC(C)(C)C)n(CCN3CCC4CNCC43)c12 ; Cl]

tert-Butyl 1-(2-{5-[(tert-butoxy)carbonyl]-octahydropyrrolo[2,3-c]pyrrol-1-yl}ethyl)-6-chloro-3-{3-[(6-fluoronaphthalen-1-yl)oxy]propyl}-7-(1,3,5-trimethyl-1H-pyrazol-4-yl)-1H-indole-2-carboxylate (175.0 mg, crude) was dissolved in THE (3.0 mL) and cooled to 0° C. To the mixture was added 4M HCl in dioxane (1.0 mL, 28.799 mmol) and the reaction was stirred at room temperature for 18 h. The crude was concentrated in vacuo to get tert-butyl 6-chloro-3-{3-[(6-fluoronaphthalen-1-yl)oxy]propyl}-1-(2-{octahydropyrrolo[2,3-c]pyrrol-1-yl}ethyl)-7-(1,3,5-trimethyl-1H-pyrazol-4-yl)-1H-indole-2-carboxylate hydrochloride (106 mg, crude) was a yellowish solid which was used to next step without further purification.

LCMS (ESI+): m/z 700.8 [M+H]⁺

### Step D

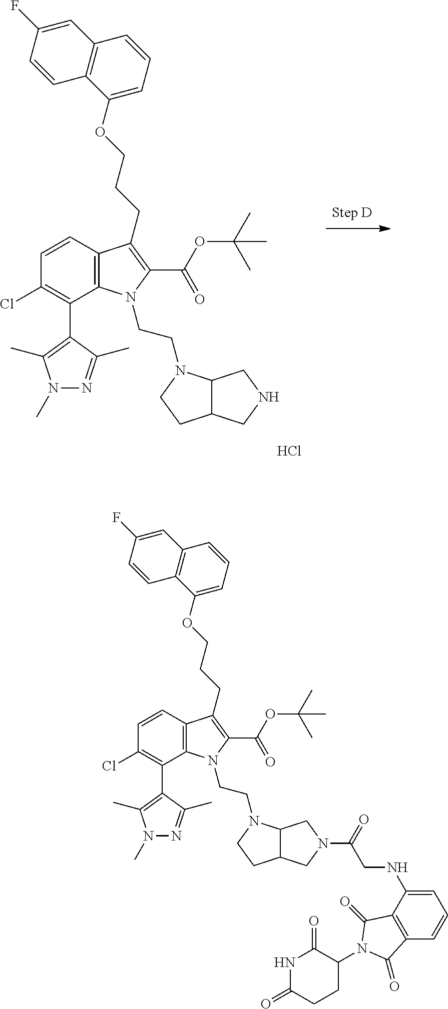 [SMILES: Cc1nn(C)c(C)c1-c1c(Cl)ccc2c(CCCOc3cccc4cc(F)ccc34)c(C(=O)OC(C)(C)C)n(CCN3CCC4CNCC43)c12 ; Cl ; Cc1nn(C)c(C)c1-c1c(Cl)ccc2c(CCCOc3cccc4cc(F)ccc34)c(C(=O)OC(C)(C)C)n(CCN3CCC4CN(C(=O)CNc5cccc6c5C(=O)N(C5CCC(=O)NC5=O)C6=O)CC43)c12]

To a solution of tert-butyl 6-chloro-3-{3-[(6-fluoronaphthalen-1-yl)oxy]propyl}-1-(2-{octahydropyrrolo[2,3-c]pyrrol-1-yl}ethyl)-7-(1,3,5-trimethyl-1H-pyrazol-4-yl)-1H-indole-2-carboxylate hydrochloride (50.0 mg, crude), 2-{[2-(2,6-dioxopiperidin-3-yl)-1,3-dioxo-2,3-dihydro-1H-isoindol-4-yl]amino}acetic acid (26.9 mg, 0.081 mmol) and HATU (51.5 mg, 0.135 mmol) in dry DMF (2.0 mL) was added DIPEA (0.035 mL, 0.203 mmol). The mixture was stirred at room temperature for 30 min. The crude was diluted with DCM and washed with brine. The organic layer was dried over Na₂SO₄, filtered and concentrated in vacuo to get 58 mg crude with tert-butyl 6-chloro-1-{2-[5-(2-{[2-(2,6-dioxopiperidin-3-yl)-1,3-dioxo-2,3-dihydro-1H-isoindol-4-yl]amino}acetyl)-octahydropyrrolo[2,3-c]pyrrol-1-yl]ethyl}-3-{3-[(6-fluoronaphthalen-1-yl)oxy]propyl}-7-(1,3,5-trimethyl-1H-pyrazol-4-yl)-1H-indole-2-carboxylate as yellow oil which was used in the next step without further purification.

LCMS (ESI+): m/z 1013.5 [M+H]⁺

### Step E

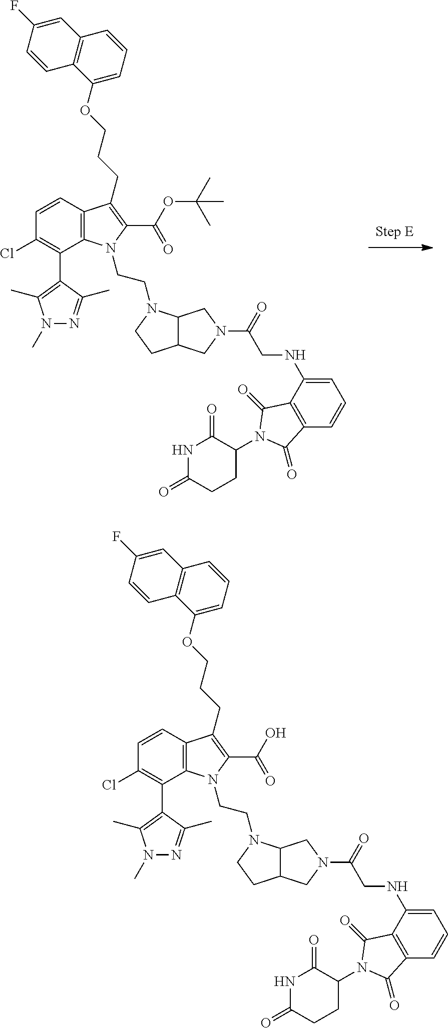 [SMILES: Cc1nn(C)c(C)c1-c1c(Cl)ccc2c(CCCOc3cccc4cc(F)ccc34)c(C(=O)OC(C)(C)C)n(CCN3CCC4CN(C(=O)CNc5cccc6c5C(=O)N(C5CCC(=O)NC5=O)C6=O)CC43)c12 ; Cc1nn(C)c(C)c1-c1c(Cl)ccc2c(CCCOc3cccc4cc(F)ccc34)c(C(=O)O)n(CCN3CCC4CN(C(=O)CNc5cccc6c5C(=O)N(C5CCC(=O)NC5=O)C6=O)CC43)c12]

To a solution of tert-butyl 6-chloro-1-{2-[5-(2-{[2-(2,6-dioxopiperidin-3-yl)-1,3-dioxo-2,3-dihydro-1H-isoindol-4-yl]amino}acetyl)-octahydropyrrolo[2,3-c]pyrrol-1-yl]ethyl}-3-{3-[(6-fluoronaphthalen-1-yl)oxy]propyl}-7-(1,3,5-trimethyl-1H-pyrazol-4-yl)-1H-indole-2-carboxylate (58 mg crude) in DCM (1.0 mL) was added TFA (1.0 mL, 13.059 mmol). The mixture was stirred for 18 h at RT. The crude was concentrated in vacuo and purified by preparative HPLC (C18, H₂O:MeCN+0.1% FA) to obtain 6-chloro-1-{2-[5-(2-{[2-(2,6-dioxopiperidin-3-yl)-1,3-dioxo-2,3-dihydro-1H-isoindol-4-yl]amino}acetyl)-octahydropyrrolo[2,3-c]pyrrol-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (27.2 mg, 0.028 mmol, yield over 51%) as an yellow solid.

LCMS (ESI+): m/z 957.4 [M+H]⁺

¹H NMR (500 MHz, DMSO) δ 10.76 (s, 1H), 8.22 (dd, J=9.2, 5.9 Hz, 1H), 7.66 (s, 1H), 7.62-7.49 (m, 2H), 7.45-7.36 (m, 2H), 7.34-7.28 (m, 1H), 7.19 (s, 1H), 7.09-6.94 (m, 3H), 6.83 (s, 1H), 5.02 (dd, J=12.7, 5.3 Hz, 1H), 4.40-4.30 (m, 1H), 4.20 (t, J=6.1 Hz, 2H), 4.16-4.06 (m, 1H), 4.05-3.94 (m, 2H), 3.78-3.73 (m, 3H), 3.71-3.60 (m, 1H), 3.58-3.47 (m, 1H), 3.35-3.18 (m, 4H), 2.67-2.52 (m, 2H), 2.19 (p, J=7.2 Hz, 2H), 2.13-1.86 (m, 11H), 1.51 (s, 1H), 1.38-1.25 (m, 1H).

2 protons in aliphatic area overlap with water and 1 proton with DMSO.

### Example 70. 6-Chloro-1-{2-[5-(2-{[2-(2,6-dioxopiperidin-3-yl)-1-oxo-2,3-dihydro-1H-isoindol-4-yl]oxy}acetyl)-octahydropyrrolo[2,3-c]pyrrol-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (301)

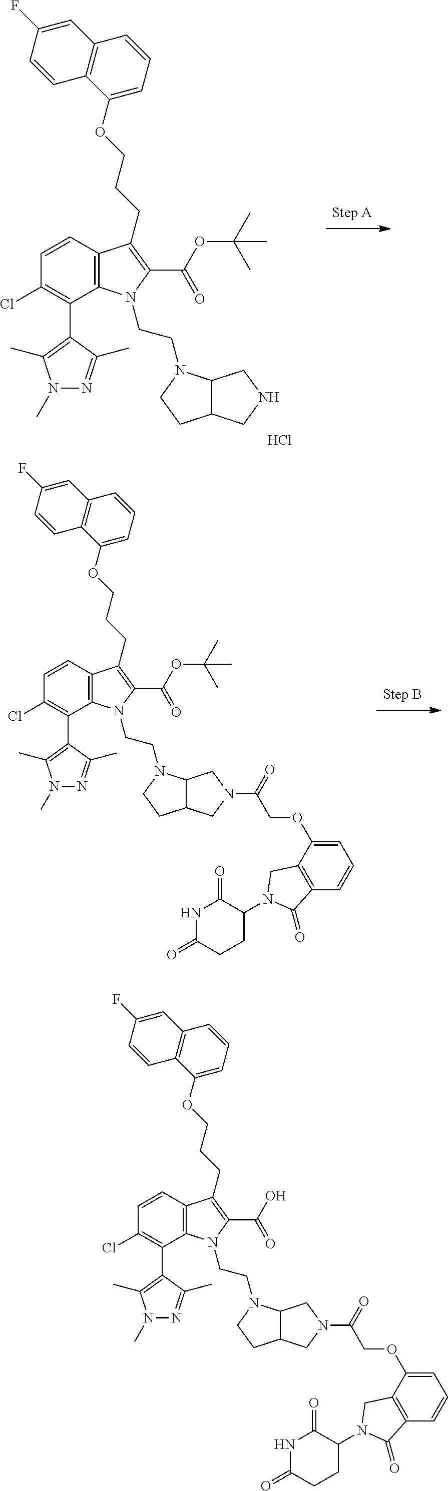 [SMILES: Cc1nn(C)c(C)c1-c1c(Cl)ccc2c(CCCOc3cccc4cc(F)ccc34)c(C(=O)OC(C)(C)C)n(CCN3CCC4CNCC43)c12 ; Cl ; Cc1nn(C)c(C)c1-c1c(Cl)ccc2c(CCCOc3cccc4cc(F)ccc34)c(C(=O)OC(C)(C)C)n(CCN3CCC4CN(C(=O)COc5cccc6c5CN(C5CCC(=O)NC5=O)C6=O)CC43)c12 ; Cc1nn(C)c(C)c1-c1c(Cl)ccc2c(CCCOc3cccc4cc(F)ccc34)c(C(=O)O)n(CCN3CCC4CN(C(=O)COc5cccc6c5CN(C5CCC(=O)NC5=O)C6=O)CC43)c12]

### Step A

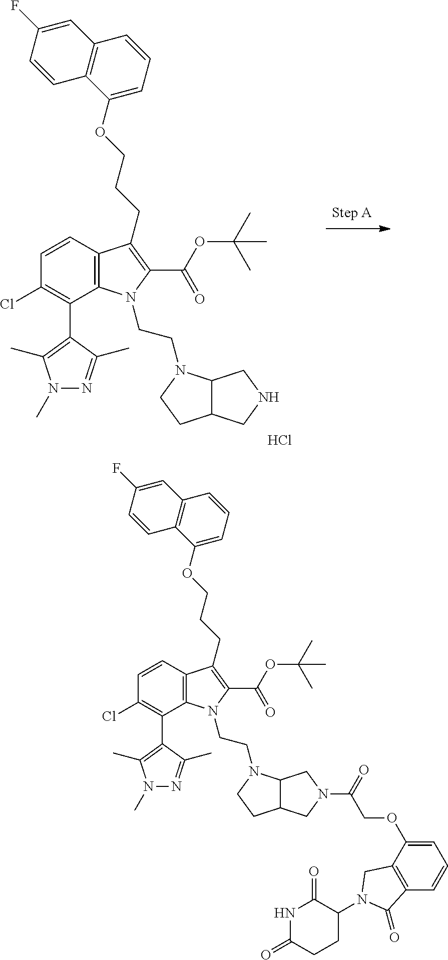 [SMILES: Cc1nn(C)c(C)c1-c1c(Cl)ccc2c(CCCOc3cccc4cc(F)ccc34)c(C(=O)OC(C)(C)C)n(CCN3CCC4CNCC43)c12 ; Cl ; Cc1nn(C)c(C)c1-c1c(Cl)ccc2c(CCCOc3cccc4cc(F)ccc34)c(C(=O)OC(C)(C)C)n(CCN3CCC4CN(C(=O)COc5cccc6c5CN(C5CCC(=O)NC5=O)C6=O)CC43)c12]

To a solution of tert-butyl 6-chloro-3-{3-[(6-fluoronaphthalen-1-yl)oxy]propyl}-1-(2-{octahydropyrrolo[2,3-c]pyrrol-1-yl}ethyl)-7-(1,3,5-trimethyl-1H-pyrazol-4-yl)-1H-indole-2-carboxylate hydrochloride (20.0 mg, 0.027 mmol), 2-{[2-(2,6-dioxopiperidin-3-yl)-1-oxo-2,3-dihydro-1H-isoindol-4-yl]oxy}acetic acid (10.3 mg, 0.032 mmol) and HATU (20.6 mg, 0.054 mmol) in dry DMF (2.0 mL) was added DIPEA (0.014 mL, 0.081 mmol). The mixture was stirred at room temperature for 30 min. The crude was diluted with DCM and washed with brine. The organic layer was dried over Na₂SO₄, filtered and concentrated in vacuo to get 33 mg crude with tert-butyl 6-chloro-1-{2-[5-(2-{[2-(2,6-dioxopiperidin-3-yl)-1-oxo-2,3-dihydro-1H-isoindol-4-yl]oxy}acetyl)-octahydropyrrolo[2,3-c]pyrrol-1-yl]ethyl}-3-{3-[(6-fluoronaphthalen-1-yl)oxy]propyl}-7-(1,3,5-trimethyl-1H-pyrazol-4-yl)-1H-indole-2-carboxylate as yellow oil which was used in the next step without further purification.

LCMS (ESI+): m/z 1000.5 [M+H]⁺

### Step B

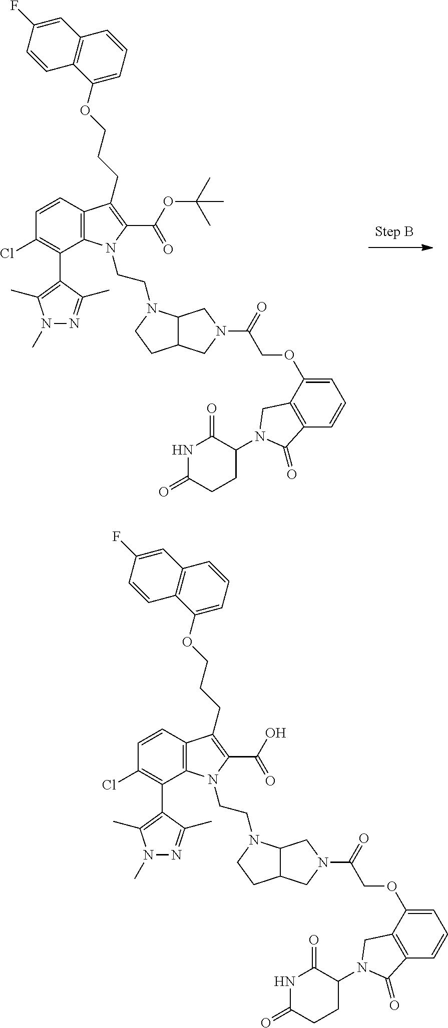 [SMILES: Cc1nn(C)c(C)c1-c1c(Cl)ccc2c(CCCOc3cccc4cc(F)ccc34)c(C(=O)OC(C)(C)C)n(CCN3CCC4CN(C(=O)COc5cccc6c5CN(C5CCC(=O)NC5=O)C6=O)CC43)c12 ; Cc1nn(C)c(C)c1-c1c(Cl)ccc2c(CCCOc3cccc4cc(F)ccc34)c(C(=O)O)n(CCN3CCC4CN(C(=O)COc5cccc6c5CN(C5CCC(=O)NC5=O)C6=O)CC43)c12]

To a solution of tert-butyl 6-chloro-1-{2-[5-(2-{[2-(2,6-dioxopiperidin-3-yl)-1-oxo-2,3-dihydro-1H-isoindol-4-yl]oxy}acetyl)-octahydropyrrolo[2,3-c]pyrrol-1-yl]ethyl}-3-{3-[(6-fluoronaphthalen-1-yl)oxy]propyl}-7-(1,3,5-trimethyl-1H-pyrazol-4-yl)-1H-indole-2-carboxylate (33 mg, crude) in DCM (0.500 mL) was added TFA (0.500 mL, 6.529 mmol). The mixture was stirred for 18 h at RT. The crude was concentrated in vacuo and purified by preparative HPLC (C18, H₂O:MeCN+0.1% FA) to obtain 6-chloro-1-{2-[5-(2-{[2-(2,6-dioxopiperidin-3-yl)-1-oxo-2,3-dihydro-1H-isoindol-4-yl]oxy}acetyl)-octahydropyrrolo[2,3-c]pyrrol-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (10.5 mg, 0.011 mmol, 40% over 2 steps) as a white solid.

LCMS (ESI+): m/z 942.25 [M+H]⁺

¹H NMR (500 MHz, DMSO, 353 K) δ 10.65 (s, 1H), 8.26 (dd, J=9.3, 5.9 Hz, 1H), 7.66 (d, J=8.6 Hz, 1H), 7.59 (dd, J=10.4, 2.6 Hz, 1H), 7.49-7.40 (m, 3H), 7.36-7.30 (m, 2H), 7.24-7.09 (m, 2H), 6.88 (dd, J=5.7, 3.0 Hz, 1H), 5.06 (dd, J=13.0, 5.2 Hz, 1H), 4.89-4.73 (m, 2H), 4.47-4.28 (m, 3H), 4.24 (t, J=6.3 Hz, 2H), 4.20-4.09 (m, 1H), 3.76 (d, J=4.0 Hz, 3H), 3.32-3.21 (m, 4H), 2.89 (ddd, J=17.3, 13.4, 5.5 Hz, 1H), 2.82-2.70 (m, 2H), 2.67-2.58 (m, 1H), 2.50-2.40 (m, 2H), 2.23 (p, J=6.9 Hz, 2H), 2.17-2.10 (m, 1H), 2.09-1.99 (m, 5H), 1.97-1.88 (m, 4H), 1.54-1.44 (m, 1H), 1.38-1.28 (m, 1H).

2 protons in aliphatic area overlap with water

### Example 71: 6-Chloro-1-(2-(7-(2-((2-(2,6-dioxopiperidin-3-yl)-1-oxoisoindolin-4-yl)oxy)acetyl)-2,7-diazaspiro[3.5]nonan-2-yl)ethyl)-3-(3-((6-fluoronaphthalen-1-yl)oxy)propyl)-7-(1,3,5-trimethyl-1H-pyrazol-4-yl)-1H-indole-2-carboxylic acid (302)

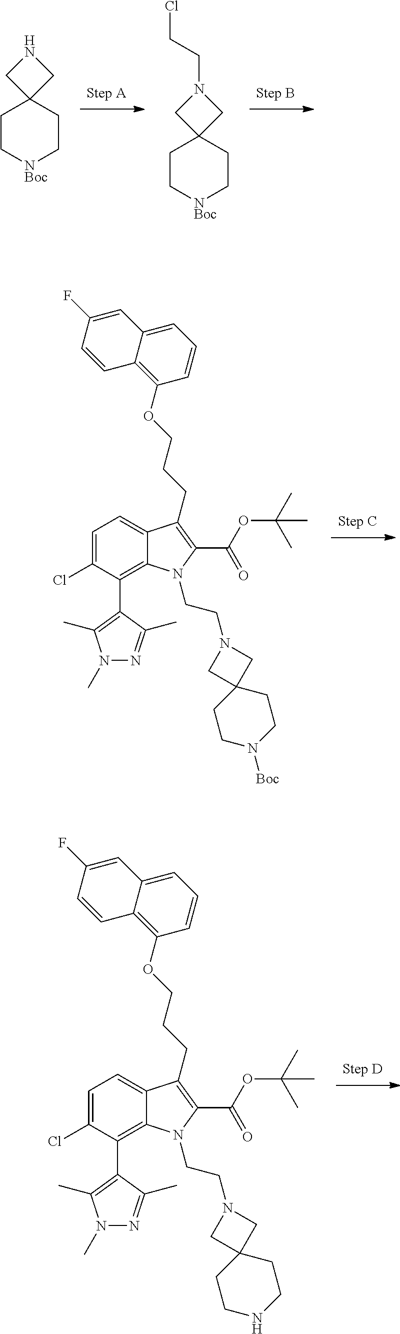

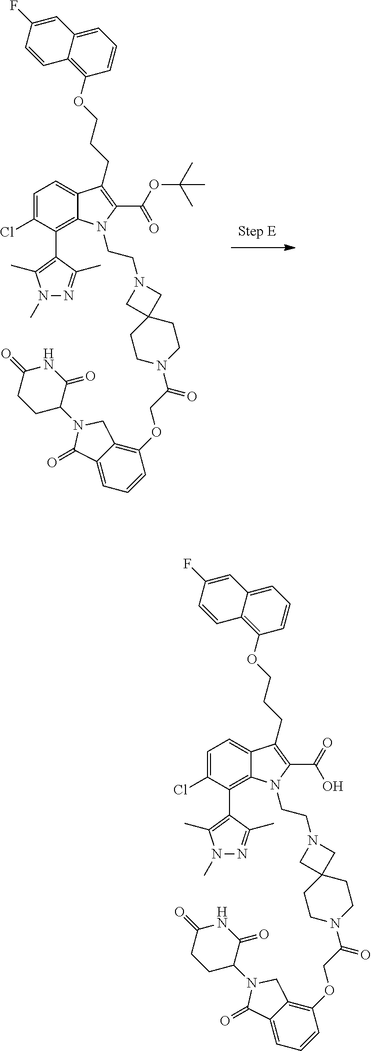 [SMILES: Cc1nn(C)c(C)c1-c1c(Cl)ccc2c(CCCOc3cccc4cc(F)ccc34)c(C(=O)OC(C)(C)C)n(CCN3CC4(CCN(C(=O)COc5cccc6c5CN(C5CCC(=O)NC5=O)C6=O)CC4)C3)c12 ; Cc1nn(C)c(C)c1-c1c(Cl)ccc2c(CCCOc3cccc4cc(F)ccc34)c(C(=O)O)n(CCN3CC4(CCN(C(=O)COc5cccc6c5CN(C5CCC(=O)NC5=O)C6=O)CC4)C3)c12]

### Step A

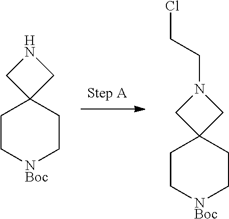 [SMILES: CC(C)(C)OC(=O)N1CCC2(CC1)CNC2 ; CC(C)(C)OC(=O)N1CCC2(CC1)CN(CCCl)C2]

To a suspension of K₂CO₃ (1.53 g, 11.05 mmol) in dry MeCN (4.5 mL) was added 1-bromo-2-chloroethane (0.368 mL, 4.41 mmol). The mixture was allowed to warm up to 70° C. and then, tert-butyl 2,7-diazaspiro[3.5]nonane-7-carboxylate (100.0 mg, 0.441 mmol) in suspension in N,N-dimethylformamide (0.5 mL) was added slowly. The reaction was stirred for 1 hour and then, water was added. Aqueous layer was extracted twice with EtOAc. Organics were washed with an aqueous solution of lithium chloride (10%) and brine, dried over anhydrous Na₂SO₄, and concentrated under reduced pressure. Crude was purified by column chromatography (SiO₂, 0-15% MeOH in EtOAc) to obtain tert-butyl 2-(2-chloroethyl)-2,7-diazaspiro[3.5]nonane-7-carboxylate (75 mg, 0.260 mmol, 59%) as a colorless oil.

### Step B

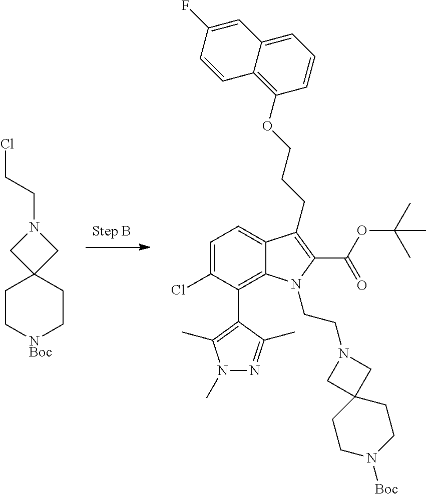 [SMILES: CC(C)(C)OC(=O)N1CCC2(CC1)CN(CCCl)C2 ; Cc1nn(C)c(C)c1-c1c(Cl)ccc2c(CCCOc3cccc4cc(F)ccc34)c(C(=O)OC(C)(C)C)n(CCN3CC4(CCN(C(=O)OC(C)(C)C)CC4)C3)c12]

tert-Butyl 6-chloro-3-(3-((6-fluoronaphthalen-1-yl)oxy)propyl)-7-(1,3,5-trimethyl-1H-pyrazol-4-yl)-1H-indole-2-carboxylate (100 mg, 0.178 mmol) and tert-butyl 2-(2-chloroethyl)-2,7-diazaspiro[3.5]nonane-7-carboxylate (51.4 mg, 0.178 mmol) were dissolved in N,N-dimethylformamide (4 mL). Then, KI (29.5 mg, 0.178 mmol) was added followed by Cs₂CO₃ (174.0 mg, 0.534 mol). The mixture was stirred at 70° C. for 12 hours. The reaction was quenched with water and aqueous layer was extracted once with EtOAc. Combined organics were washed three times with brine and water, dried over Na₂SO₄ and concentrated under vacuum. Crude was purified by column chromatography (SiO₂, 0-15% MeOH in EtOAc) to obtain tert-butyl 2-(2-(2-(tert-butoxycarbonyl)-6-chloro-3-(3-((6-fluoronaphthalen-1-yl)oxy)propyl)-7-(1,3,5-trimethyl-1H-pyrazol-4-yl)-1H-indol-1-yl)ethyl)-2,7-diazaspiro[3.5]nonane-7-carboxylate (140 mg, 0.172 mmol, 97%) as a white solid.

LCMS (ESI⁺): 814.1 m/z [M+H]⁺

### Step C

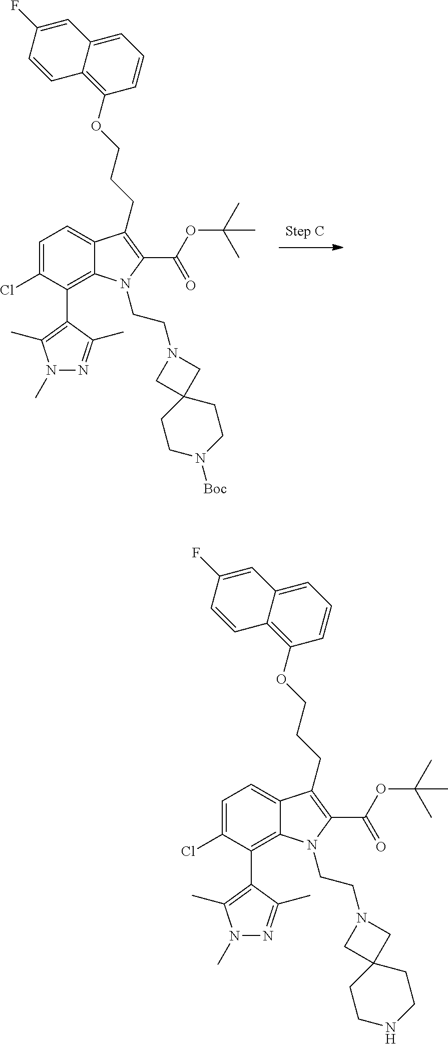 [SMILES: Cc1nn(C)c(C)c1-c1c(Cl)ccc2c(CCCOc3cccc4cc(F)ccc34)c(C(=O)OC(C)(C)C)n(CCN3CC4(CCN(C(=O)OC(C)(C)C)CC4)C3)c12 ; Cc1nn(C)c(C)c1-c1c(Cl)ccc2c(CCCOc3cccc4cc(F)ccc34)c(C(=O)OC(C)(C)C)n(CCN3CC4(CCNCC4)C3)c12]

tert-Butyl 2-(2-(2-(tert-butoxycarbonyl)-6-chloro-3-(3-((6-fluoronaphthalen-1-yl)oxy)propyl)-7-(1,3,5-trimethyl-1H-pyrazol-4-yl)-1H-indol-1-yl)ethyl)-2,7-diazaspiro[3.5]nonane-7-carboxylate (45.00 mg, 0.055 mmol) was dissolved in DCM (1 mL) and TFA (63.85 μL, 0.829 mmol) was added at 0° C. and the reaction was stirred for 3 hours. Upon completion the reaction mixture was concentrated under vacuum and crude (tert-butyl 1-(2-(2,7-diazaspiro[3.5]nonan-2-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate) was used without further purification.

LCMS (ESI⁺): 714.2 m/z [M+H]⁺

### Step D

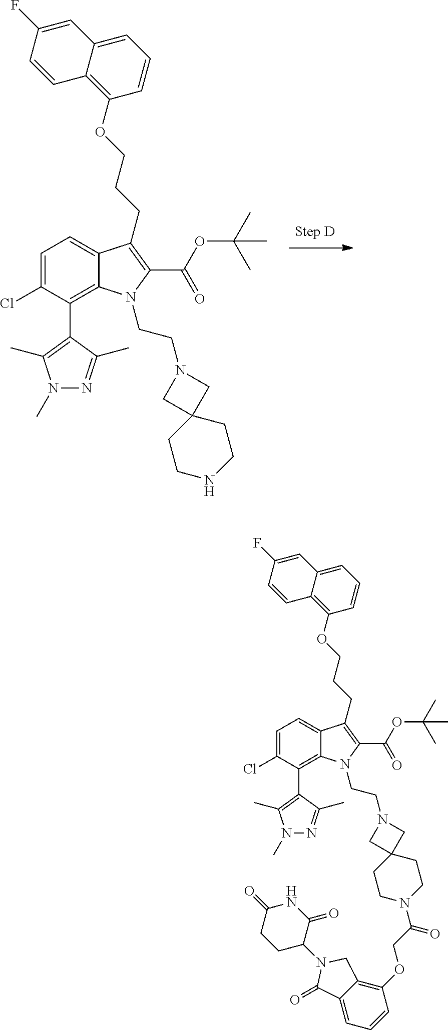 [SMILES: Cc1nn(C)c(C)c1-c1c(Cl)ccc2c(CCCOc3cccc4cc(F)ccc34)c(C(=O)OC(C)(C)C)n(CCN3CC4(CCNCC4)C3)c12 ; Cc1nn(C)c(C)c1-c1c(Cl)ccc2c(CCCOc3cccc4cc(F)ccc34)c(C(=O)OC(C)(C)C)n(CCN3CC4(CCN(C(=O)COc5cccc6c5CN(C5CCC(=O)NC5=O)C6=O)CC4)C3)c12]

To a solution of 2-((2-(2,6-dioxopiperidin-3-yl)-1-oxoisoindolin-4-yl)oxy)acetic acid (15.99 mg, 0.050 mmol) in dichloromethane (1 mL) was added HATU (22.92 mg, 0.060 mmol), tert-butyl 1-(2-(2,7-diazaspiro[3.5]nonan-2-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate (39.47 mg, 0.055 mmol), then, DIPEA (42.9 μL, 0.251 mmol) and the reaction mixture was stirred 16 hours at room temperature. Celite® was added, the reaction mixture was concentrated and purified by reverse phase chromatography (C18, H₂O:MeCN+0.1% FA) to afford tert-butyl 6-chloro-1-(2-(7-(2-((2-(2,6-dioxopiperidin-3-yl)-1-oxoisoindolin-4-yl)oxy)acetyl)-2,7-diazaspiro[3.5]nonan-2-yl)ethyl)-3-(3-((6-fluoronaphthalen-1-yl)oxy)propyl)-7-(1,3,5-trimethyl-1H-pyrazol-4-yl)-1H-indole-2-carboxylate (33.1 mg, 0.032 mmol, 65%) as a white solid.

LCMS (ESI+): 1012.3 m/z [M+H]⁺

### Step E

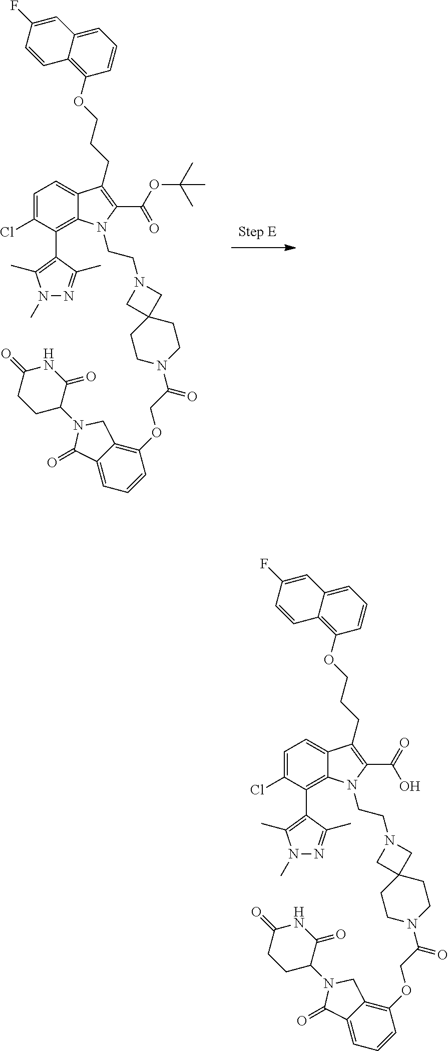 [SMILES: Cc1nn(C)c(C)c1-c1c(Cl)ccc2c(CCCOc3cccc4cc(F)ccc34)c(C(=O)OC(C)(C)C)n(CCN3CC4(CCN(C(=O)COc5cccc6c5CN(C5CCC(=O)NC5=O)C6=O)CC4)C3)c12 ; Cc1nn(C)c(C)c1-c1c(Cl)ccc2c(CCCOc3cccc4cc(F)ccc34)c(C(=O)O)n(CCN3CC4(CCN(C(=O)COc5cccc6c5CN(C5CCC(=O)NC5=O)C6=O)CC4)C3)c12]

To a solution of tert-butyl 6-chloro-1-(2-(7-(2-((2-(2,6-dioxopiperidin-3-yl)-1-oxoisoindolin-4-yl)oxy)acetyl)-2,7-diazaspiro[3.5]nonan-2-yl)ethyl)-3-(3-((6-fluoronaphthalen-1-yl)oxy)propyl)-7-(1,3,5-trimethyl-1H-pyrazol-4-yl)-1H-indole-2-carboxylate (30.00 mg, 0.030 mmol) in DCM (0.591 mL) at 0° C. was added TFA (0.068 mL). The mixture was allowed to warm up to room temperature and stirred overnight. Then, more TFA (0.230 mL) was added and mixture was stirred for 2 hours. The mixture was concentrated under vacuum and crude was purified by reverse phase (C18, H₂O:MeCN+0.1% FA) to obtain 6-chloro-1-(2-(7-(2-((2-(2,6-dioxopiperidin-3-yl)-1-oxoisoindolin-4-yl)oxy)acetyl)-2,7-diazaspiro[3.5]nonan-2-yl)ethyl)-3-(3-((6-fluoronaphthalen-1-yl)oxy)propyl)-7-(1,3,5-trimethyl-1H-pyrazol-4-yl)-1H-indole-2-carboxylic acid (7.5 mg, 0.008 mmol, 26%) as a white solid.

LCMS (ESI+): 958 m/z [M+H]⁺

¹H NMR (400 MHz, DMSO) δ 10.98 (s, 1H), 8.23 (dd, J=9.2, 5.9 Hz, 1H), 7.67 (d, J=8.5 Hz, 1H), 7.63 (dd, J=10.4, 2.7 Hz, 1H), 7.44-7.39 (m, 3H), 7.36 (td, J=8.9, 2.6 Hz, 1H), 7.30 (d, J=7.4 Hz, 1H), 7.21 (d, J=8.5 Hz, 1H), 7.06 (d, J=8.2 Hz, 1H), 6.91-6.83 (m, 1H), 5.10 (dd, J=13.3, 5.1 Hz, 1H), 4.89 (s, 2H), 4.36 (d, J=17.4 Hz, 1H), 4.28-4.19 (m, 3H), 4.16-4.06 (m, 2H), 3.78 (s, 3H), 3.26-3.14 (m, 6H), 3.11-2.97 (m, 4H), 2.91 (ddd, J=18.0, 13.5, 5.4 Hz, 1H), 2.63-2.55 (m, 3H), 2.42 (td, J=13.1, 4.4 Hz, 1H), 2.19 (p, J=7.0 Hz, 2H), 2.04 (s, 3H), 2.02-1.95 (m, 1H), 1.92 (s, 3H), 1.69-1.59 (m, 2H), 1.58-1.46 (m, 2H).

### Example 72: 6-Chloro-1-(2-(6-(2-((2-(2,6-dioxopiperidin-3-yl)-1-oxoisoindolin-4-yl)oxy)acetyl)-2,6-diazaspiro[3.3]heptan-2-yl)ethyl)-3-(3-((6-fluoronaphthalen-1-yl)oxypropyl)-7-(1,3,5-trimethyl-1H-pyrazol-4-yl)-1H-indole-2-carboxylic acid (303)

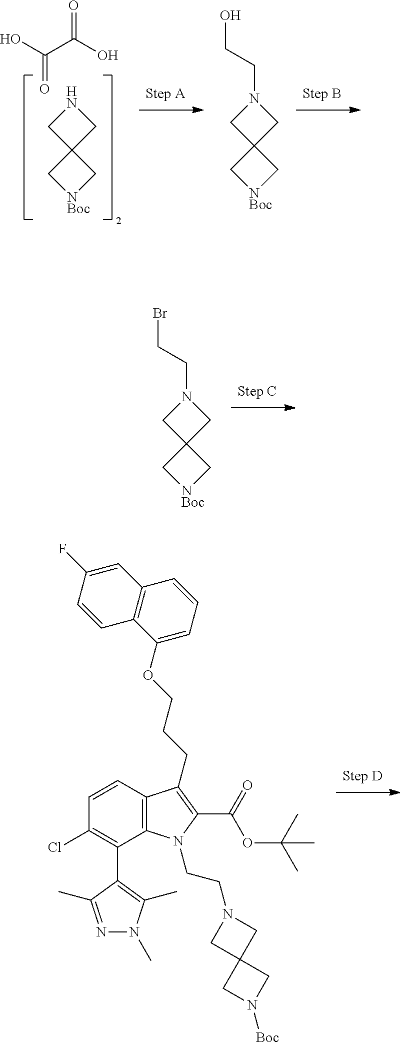

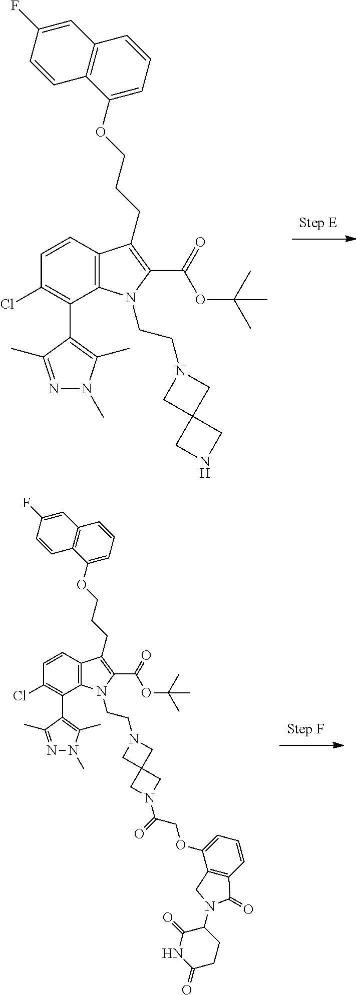

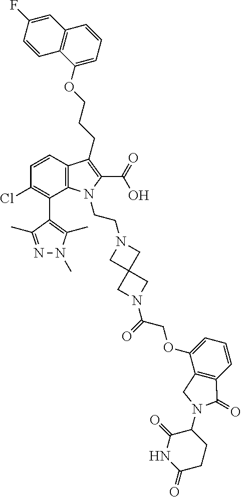 [SMILES: Cc1nn(C)c(C)c1-c1c(Cl)ccc2c(CCCOc3cccc4cc(F)ccc34)c(C(=O)O)n(CCN3CC4(C3)CN(C(=O)COc3cccc5c3CN(C3CCC(=O)NC3=O)C5=O)C4)c12]

### Step A

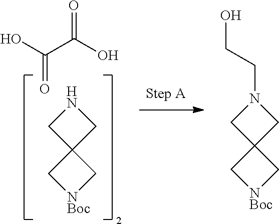

To a stirred solution of tert-butyl 2,6-diazaspiro[3.3]heptane-2-carboxylate hemioxalate (500 mg, 1.03 mmol) in MeCN (10.3 mL) were added K₂CO₃ (710 mg, 5.14 mmol) and ethylene bromohydrin (0.146 mL, 2.06 mmol), then the resultant reaction mixture was refluxed for 16 h. After complete conversion of the starting material, the reaction mixture was cooled to room temperature; filtered and concentrated under reduce pressure. The crude was purified by column chromatography (SiO₂, 0-40% MeOH in DCM) to afford tert-butyl 6-(2-hydroxyethyl)-2,6-diazaspiro[3.3]heptane-2-carboxylate (370 mg, 1.53 mmol, 61%) as a colorless sticky oil.

### Step B

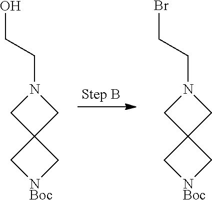 [SMILES: CC(C)(C)OC(=O)N1CC2(CN(CCO)C2)C1 ; CC(C)(C)OC(=O)N1CC2(CN(CCBr)C2)C1]

To a stirred solution of tert-butyl 6-(2-hydroxyethyl)-2,6-diazaspiro[3.3]heptane-2-carboxylate (400 mg, 1.65 mmol) in DCM (8.25 mL) were added triphenylphosphine (476 mg, 1.82 mmol) followed by the addition of carbon tetrabromide (614 mg, 1.82 mmol) and then the resultant reaction mixture was stirred at room temperature for 16 hours. After complete conversion of the starting material, the reaction mixture was concentrated under reduced pressure. The crude was purified by column chromatography (SiO₂, 0-25% MeOH in DCM) to afford the desired compound tert-butyl 6-(2-bromoethyl)-2,6-diazaspiro[3.3]heptane-2-carboxylate (150 mg, 0.491 mmol, 30%) as an off-white solid.

### Step C

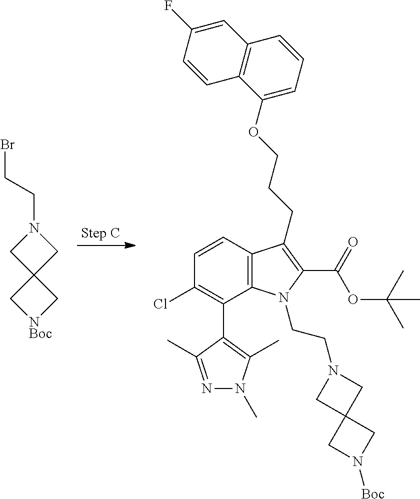 [SMILES: CC(C)(C)OC(=O)N1CC2(CN(CCBr)C2)C1 ; Cc1nn(C)c(C)c1-c1c(Cl)ccc2c(CCCOc3cccc4cc(F)ccc34)c(C(=O)OC(C)(C)C)n(CCN3CC4(C3)CN(C(=O)OC(C)(C)C)C4)c12]

To a stirred solution of tert-butyl 6-chloro-3-(3-((6-fluoronaphthalen-1-yl)oxy)propyl)-7-(1,3,5-trimethyl-1H-pyrazol-4-yl)-1H-indole-2-carboxylate (150 mg, 0.267 mmol) in DMF (5.34 mL) were added Cs₂CO₃ (261 mg, 0.801 mmol) and KI (44.3 mg, 0.267 mmol) followed by the addition of tert-butyl 6-(2-bromoethyl)-2,6-diazaspiro[3.3]heptane-2-carboxylate (89.6 mg, 0.294 mmol) and the resultant reaction mixture was stirred for 16 hours at room temperature. After complete consumption of the starting material, the reaction mixture was diluted with water and extracted twice with EtOAc. The combine organic layer was dried over Na₂SO₄ and concentrated under reduced pressure. The crude was purified by column chromatography (SiO₂, 0-10% MeOH in DCM) to afford tert-butyl 1-(2-(6-(tert-butoxycarbonyl)-2,6-diazaspiro[3.3]heptan-2-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate (100 mg, 0.127 mmol, 48%) as a sticky off-white solid.

LCMS (ESI+): m/z 786 [M+H]⁺

### Step D

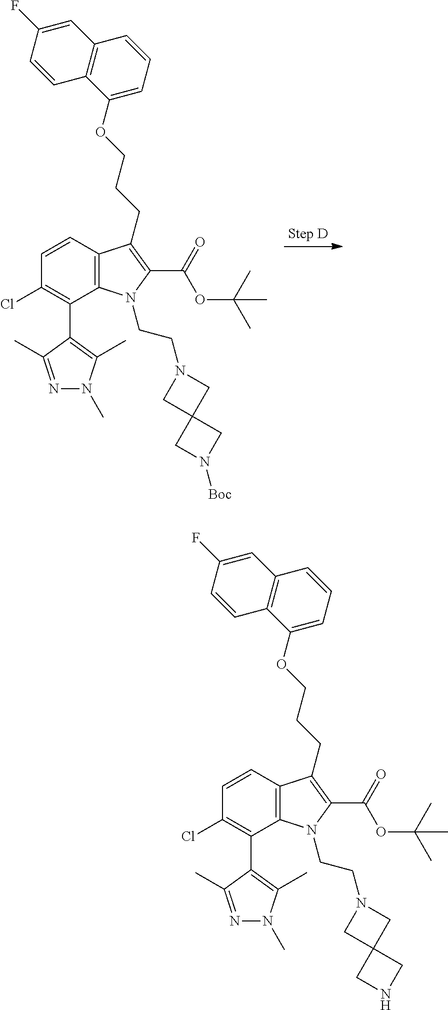 [SMILES: Cc1nn(C)c(C)c1-c1c(Cl)ccc2c(CCCOc3cccc4cc(F)ccc34)c(C(=O)OC(C)(C)C)n(CCN3CC4(C3)CN(C(=O)OC(C)(C)C)C4)c12 ; Cc1nn(C)c(C)c1-c1c(Cl)ccc2c(CCCOc3cccc4cc(F)ccc34)c(C(=O)OC(C)(C)C)n(CCN3CC4(CNC4)C3)c12]

To a stirred solution of tert-butyl 1-(2-(6-(tert-butoxycarbonyl)-2,6-diazaspiro[3.3]heptan-2-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate (150 mg, 0.19 mmol) in DCM (3.81 mL) at 0° C., TFA (0.22 mL, 2.86 mmol) was added dropwise and the resultant reaction mixture was stirred for 3 h at RT. After complete consumption of the starting material, the reaction mixture concentrated under reduced pressure to obtain the crude. The crude was triturated with cyclohexane (3 times) to afford the desired compound tert-butyl 1-(2-(2,6-diazaspiro[3.3]heptan-2-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate (140 mg, crude) as a sticky off white solid, which was used in the next step without further purification.

LCMS (ESI+): m/z 686 [M+H]⁺

### Step E

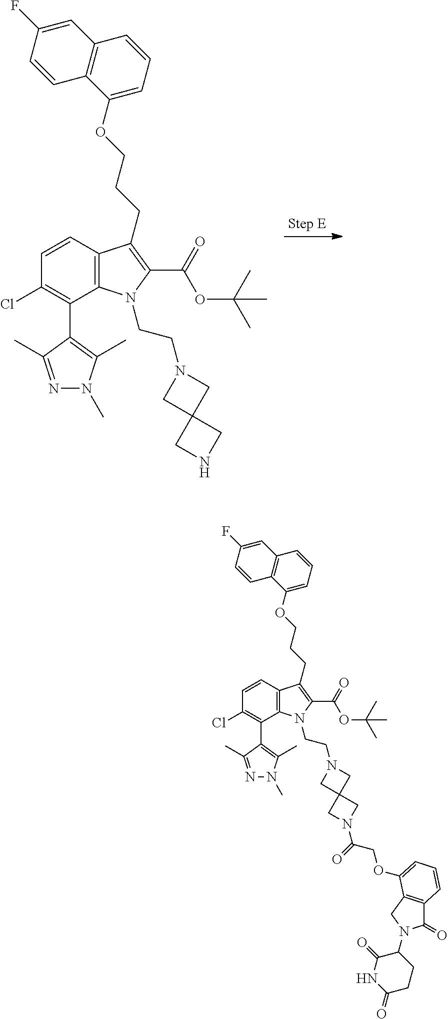 [SMILES: Cc1nn(C)c(C)c1-c1c(Cl)ccc2c(CCCOc3cccc4cc(F)ccc34)c(C(=O)OC(C)(C)C)n(CCN3CC4(CNC4)C3)c12 ; Cc1nn(C)c(C)c1-c1c(Cl)ccc2c(CCCOc3cccc4cc(F)ccc34)c(C(=O)OC(C)(C)C)n(CCN3CC4(C3)CN(C(=O)COc3cccc5c3CN(C3CCC(=O)NC3=O)C5=O)C4)c12]

To a solution of 2-((2-(2,6-dioxopiperidin-3-yl)-1-oxoisoindolin-4-yl)oxy)acetic acid (30.0 mg, 0.094 mmol) in DCM (1.9 mL) were added HATU (43.0 mg, 0.113 mmol) and tert-butyl 1-(2-(2,6-diazaspiro[3.3]heptan-2-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate (81.3 mg, 0.104 mmol) followed by the addition of DIPEA (0.082 mL, 0.471 mmol) and the reaction mixture was stirred for 16 h at RT. After complete consumption of the starting material the reaction mixture was concentrated under reduced pressure. Then the crude was purified by reverse phase column chromatography (C18, H₂O:MeCN+0.1% FA) to afford tert-butyl 6-chloro-1-(2-(6-(2-((2-(2,6-dioxopiperidin-3-yl)-1-oxoisoindolin-4-yl)oxy)acetyl)-2,6-diazaspiro[3.3]heptan-2-yl)ethyl)-3-(3-((6-fluoronaphthalen-1-yl)oxy)propyl)-7-(1,3,5-trimethyl-1H-pyrazol-4-yl)-1H-indole-2-carboxylate as a white solid (12.0 mg, 0.012 mmol, 11%).

LCMS (ESI+): m/z 986 [M+H]⁺

### Step F

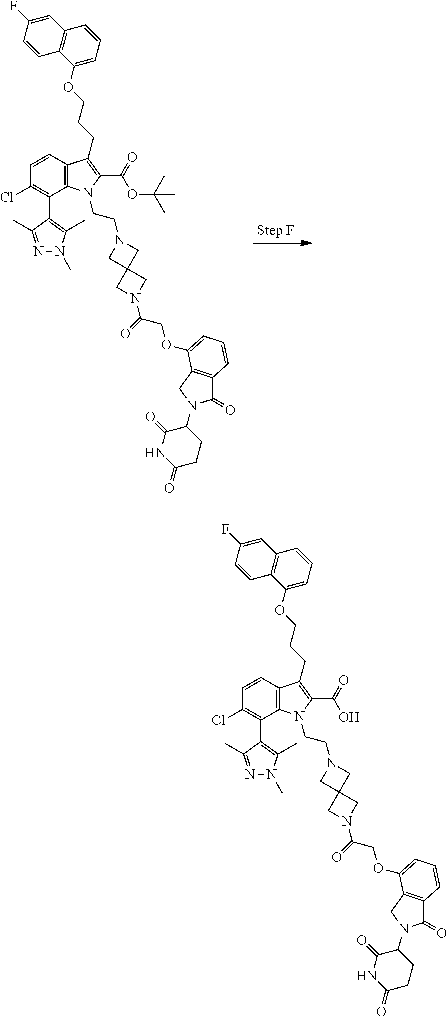 [SMILES: Cc1nn(C)c(C)c1-c1c(Cl)ccc2c(CCCOc3cccc4cc(F)ccc34)c(C(=O)OC(C)(C)C)n(CCN3CC4(C3)CN(C(=O)COc3cccc5c3CN(C3CCC(=O)NC3=O)C5=O)C4)c12 ; Cc1nn(C)c(C)c1-c1c(Cl)ccc2c(CCCOc3cccc4cc(F)ccc34)c(C(=O)O)n(CCN3CC4(C3)CN(C(=O)COc3cccc5c3CN(C3CCC(=O)NC3=O)C5=O)C4)c12]

To a solution of tert-butyl 6-chloro-1-(2-(6-(2-((2-(2,6-dioxopiperidin-3-yl)-1-oxoisoindolin-4-yl)oxy)acetyl)-2,6-diazaspiro[3.3]heptan-2-yl)ethyl)-3-(3-((6-fluoronaphthalen-1-yl)oxy)propyl)-7-(1,3,5-trimethyl-1H-pyrazol-4-yl)-1H-indole-2-carboxylate (22.0 mg, 0.022 mmol) in DCM (0.791 mL) at 0° C. was added FA (0.492 mL, 13.0 mmol). The resultant reaction mixture was stirred overnight at 80° C. After that it was concentrated under reduced pressure and the crude was purified by reverse phase (C18, H₂O:MeCN+0.1% FA) to obtain 6-chloro-1-(2-(6-(2-((2-(2,6-dioxopiperidin-3-yl)-1-oxoisoindolin-4-yl)oxy)acetyl)-2,6-diazaspiro[3.3]heptan-2-yl)ethyl)-3-(3-((6-fluoronaphthalen-1-yl)oxy)propyl)-7-(1,3,5-trimethyl-1H-pyrazol-4-yl)-1H-indole-2-carboxylic acid (4.0 mg, 0.004 mmol, 18%) as a white solid.

LCMS (ESI+): m/z 930 [M+H]⁺

¹H NMR (400 MHz, DMSO) δ 10.99 (s, 1H), 8.21 (dd, J=9.3, 5.9 Hz, 1H), 7.70 (d, J=8.5 Hz, 1H), 7.63 (dd, J=10.3, 2.7 Hz, 1H), 7.48-7.37 (m, 3H), 7.39-7.29 (m, 2H), 7.22 (d, J=8.5 Hz, 1H), 7.09 (d, J=8.2 Hz, 1H), 6.90-6.83 (m, 1H), 5.11 (dd, J=13.3, 5.1 Hz, 1H), 4.66 (s, 2H), 4.39 (d, J=17.4 Hz, 1H), 4.25 (d, J=17.4 Hz, 1H), 4.23-4.16 (m, 5H), 4.13-3.96 (m, 1H), 3.88 (s, 2H), 3.76 (s, 3H), 3.27-3.18 (m, 2H), 3.15-3.05 (m, 4H), 2.98-2.80 (m, 1H), 2.63-2.53 (m, 1H), 2.45-2.38 (m, 1H), 2.30-2.13 (m, 4H), 2.04-1.96 (m, 4H), 1.88 (s, 3H).

### Example 73: 6-Chloro-1-(2-(6-(2-((2-(2,6-dioxopiperidin-3-yl)-1-oxoisoindolin-4-yl)oxy)acetyl)-2,6-diazaspiro[3.5]nonan-2-yl)ethyl)-3-(3-((6-fluoronaphthalen-1-yl)oxy)propyl)-7-(1,3,5-trimethyl-1H-pyrazol-4-yl)-1H-indole-2-carboxylic acid (304)

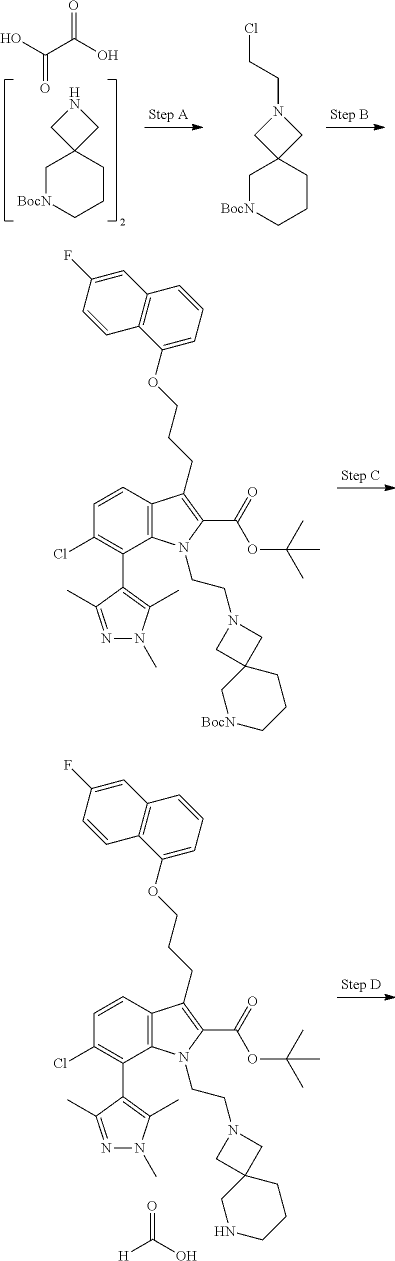

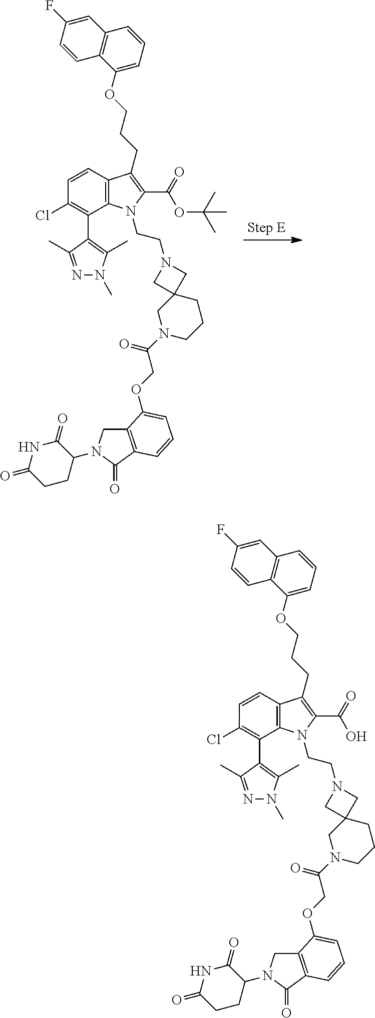 [SMILES: Cc1nn(C)c(C)c1-c1c(Cl)ccc2c(CCCOc3cccc4cc(F)ccc34)c(C(=O)OC(C)(C)C)n(CCN3CC4(CCCN(C(=O)COc5cccc6c5CN(C5CCC(=O)NC5=O)C6=O)C4)C3)c12 ; Cc1nn(C)c(C)c1-c1c(Cl)ccc2c(CCCOc3cccc4cc(F)ccc34)c(C(=O)O)n(CCN3CC4(CCCN(C(=O)COc5cccc6c5CN(C5CCC(=O)NC5=O)C6=O)C4)C3)c12]

### Step A

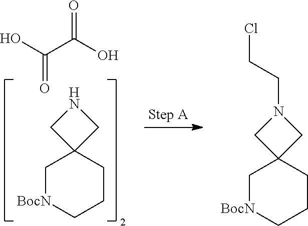

To a suspension of K₂CO₃ (1.39 g, 10.0 mmol) in dry MeCN (4.0 mL) was added 1-bromo-2-chloroethane (0.500 mL, 6.03 mmol). The mixture was allowed to warm up to 70° C. and then, tert-butyl 2,6-diazaspiro[3.5]nonane-6-carboxylate hemioxalate (218.0 mg, 0.40 mmol) was added portionwise over a period of 15 minutes. The reaction was stirred for 1 hour and the solid was filtered off washed with EtOAc and the filtrate was concentrated to dryness. Crude was purified by column chromatography (SiO₂, 0-10% of MeOH in EtOAc) to obtain tert-butyl 2-(2-chloroethyl)-2,6-diazaspiro[3.5]nonane-6-carboxylate (114 mg, 0.395 mmol, 98%) as a colorless oil.

### Step B

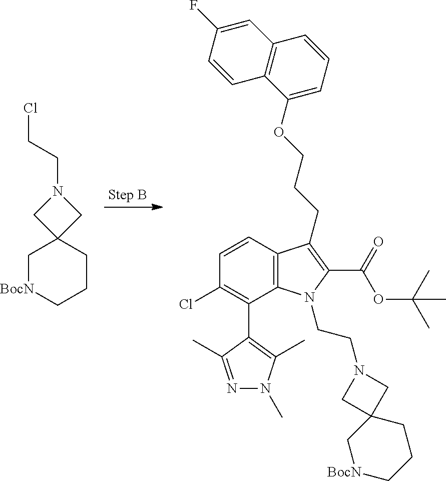 [SMILES: CC(C)(C)OC(=O)N1CCCC2(CN(CCCl)C2)C1 ; Cc1nn(C)c(C)c1-c1c(Cl)ccc2c(CCCOc3cccc4cc(F)ccc34)c(C(=O)OC(C)(C)C)n(CCN3CC4(CCCN(C(=O)OC(C)(C)C)C4)C3)c12]

tert-Butyl 6-chloro-3-(3-((6-fluoronaphthalen-1-yl)oxy)propyl)-7-(1,3,5-trimethyl-1H-pyrazol-4-yl)-1H-indole-2-carboxylate (100 mg, 0.178 mmol) and tert-butyl 2-(2-chloroethyl)-2,6-diazaspiro[3.5]nonane-6-carboxylate (56.5 mg, 0.195 mmol) were dissolved in DMF (4 mL). Then, KI (29.5 mg, 178.0 μmol) was added followed by Cs₂CO₃ (174.0 mg, 0.534 mmol). The mixture was stirred at 70° C. for 12 hours. The reaction was quenched with water and aqueous layer was extracted once with EtOAc. Combined organics were washed three times with brine and water, dried over Na₂SO₄ and concentrated under vacuum. Crude was purified by column chromatography (SiO₂, 0-15% MeOH in EtOAc) to obtain pure tert-butyl 2-(2-(2-(tert-butoxycarbonyl)-6-chloro-3-(3-((6-fluoronaphthalen-1-yl)oxy)propyl)-7-(1,3,5-trimethyl-1H-pyrazol-4-yl)-1H-indol-1-yl)ethyl)-2,6-diazaspiro[3.5]nonane-6-carboxylate (142 mg, 0.174 mol, 98%) as a white solid.

LCMS (ESI⁺): m/z 814 [M+H]⁺

### Step C

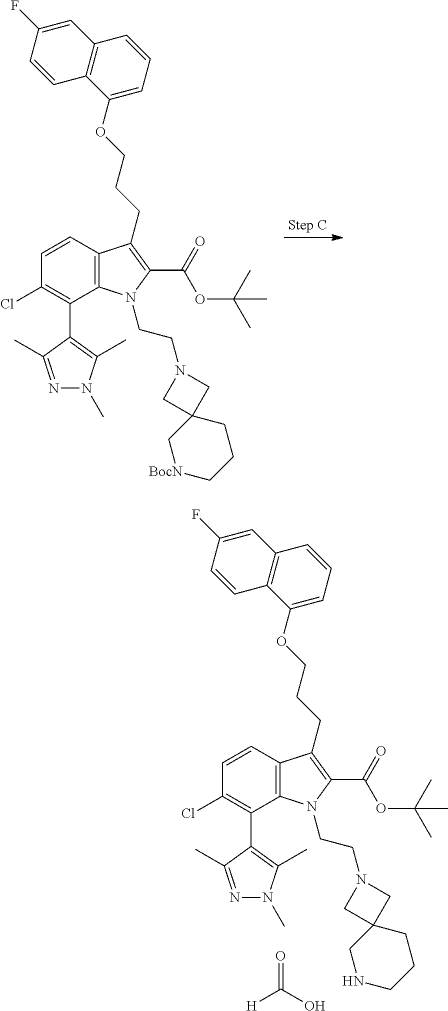

To a solution of tert-butyl 2-(2-(2-(tert-butoxycarbonyl)-6-chloro-3-(3-((6-fluoronaphthalen-1-yl)oxy)propyl)-7-(1,3,5-trimethyl-1H-pyrazol-4-yl)-1H-indol-1-yl)ethyl)-2,6-diazaspiro[3.5]nonane-6-carboxylate (350.0 mg, 430 mmol) in DCM (10 mL) at 0° C. was added formic acid (10.0 mL, 258 mmol). The resultant reaction mixture was stirred overnight at room temperature. The volatiles were removed in vacuo and the crude residue was triturated three times with Et₂O to afford tert-butyl 1-(2-(2,6-diazaspiro[3.5]nonan-2-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate formate (280 mg, 368 mmol, 85%) as a white solid.

LCMS (ESI⁺): m/z 714.1 [M+H]⁺

### Step D

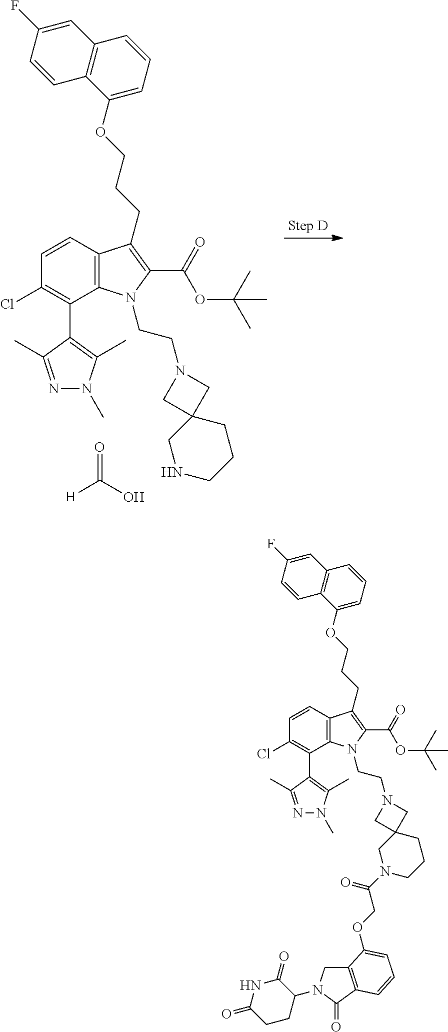

To a solution of 2-((2-(2,6-dioxopiperidin-3-yl)-1-oxoisoindolin-4-yl)oxy)acetic acid (13.2 mg, 0.042 mmol) in DCM (0.6 mL) was added HATU (22.92 mg, 0.060 mmol), tert-butyl 1-(2-(2,6-diazaspiro[3.5]nonan-2-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate formate (20.0 mg, 0.026 mmol), DIPEA (0.015 mL, 0.084 mmol) and the reaction mixture was stirred 1 hour at room temperature. Sat. aq. NaHCO₃ solution was added followed by EtOAc. Layers were separated and aqueous layer was extracted twice with EtOAc. Combined organic layers were washed with brine, dried over Na₂SO₄ then concentrated. The crude of tert-butyl 6-chloro-1-(2-(6-(2-((2-(2,6-dioxopiperidin-3-yl)-1-oxoisoindolin-4-yl)oxy)acetyl)-2,6-diazaspiro[3.5]nonan-2-yl)ethyl)-3-(3-((6-fluoronaphthalen-1-yl)oxy)propyl)-7-(1,3,5-trimethyl-1H-pyrazol-4-yl)-1H-indole-2-carboxylate was directly engaged in the next step without further purification.

### Step E

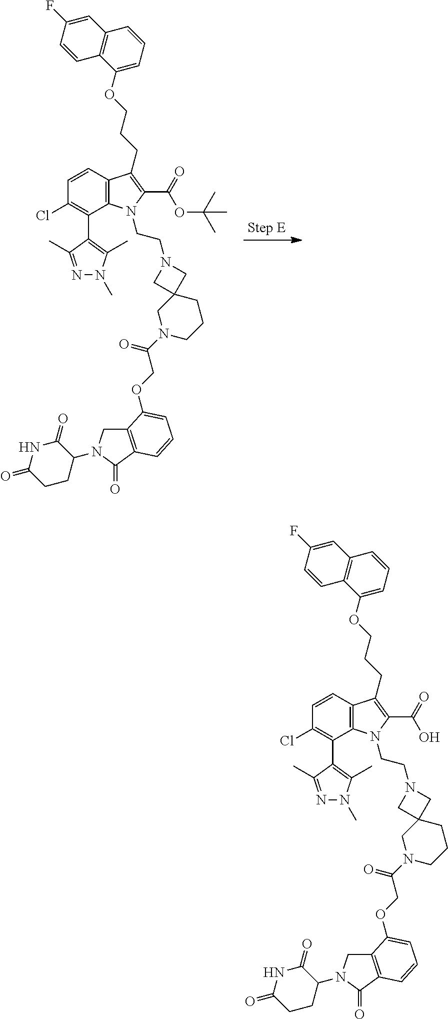 [SMILES: Cc1nn(C)c(C)c1-c1c(Cl)ccc2c(CCCOc3cccc4cc(F)ccc34)c(C(=O)OC(C)(C)C)n(CCN3CC4(CCCN(C(=O)COc5cccc6c5CN(C5CCC(=O)NC5=O)C6=O)C4)C3)c12 ; Cc1nn(C)c(C)c1-c1c(Cl)ccc2c(CCCOc3cccc4cc(F)ccc34)c(C(=O)O)n(CCN3CC4(CCCN(C(=O)COc5cccc6c5CN(C5CCC(=O)NC5=O)C6=O)C4)C3)c12]

To a solution of crude tert-butyl 6-chloro-1-(2-(6-(2-((2-(2,6-dioxopiperidin-3-yl)-1-oxoisoindolin-4-yl)oxy)acetyl)-2,6-diazaspiro[3.5]nonan-2-yl)ethyl)-3-(3-((6-fluoronaphthalen-1-yl)oxy)propyl)-7-(1,3,5-trimethyl-1H-pyrazol-4-yl)-1H-indole-2-carboxylate in DCM (0.500 mL) at 0° C. was added TFA (0.113 mL, 1.48 mmol). The mixture was allowed to warm up to room temperature and stirred overnight. The mixture was concentrated under vacuum and crude was purified by reverse phase (C18, H₂O:MeCN+0.1% FA) to obtain 6-chloro-1-(2-(6-(2-((2-(2,6-dioxopiperidin-3-yl)-1-oxoisoindolin-4-yl)oxy)acetyl)-2,6-diazaspiro[3.5]nonan-2-yl)ethyl)-3-(3-((6-fluoronaphthalen-1-yl)oxy)propyl)-7-(1,3,5-trimethyl-1H-pyrazol-4-yl)-1H-indole-2-carboxylic acid (14 mg, 0.014 mmol, 53% over 2 steps) as a white solid.

LCMS (ESI⁺): m/z 958.1 [M+H]⁺

¹H NMR (400 MHz, DMSO) 5=10.98 (s, 1H), 8.23 (dd, J=9.3, 5.9, 1H), 7.78-7.68 (m, 1H), 7.65 (dd, J=10.6, 2.7, 1H), 7.48-7.19 (m, 6H), 7.18-7.03 (m, 1H), 6.93-6.77 (m, 1H), 5.18-4.87 (m, 3H), 4.44-4.03 (m, 6H), 3.75 (s, 3H), 2.96-2.86 (m, 1H), 2.62-2.53 (m, 2H), 2.45-2.35 (m, 1H), 2.26-2.10 (m, 2H), 2.06-1.85 (m, 7H), 1.77-1.57 (m, 2H), 1.49-1.39 (m, 1H), 1.39-1.26 (m, 1H).

11 protons in aliphatic area overlap with water.

### Example 74: 6-Chloro-1-(2-(6-(2-((2-(2,6-dioxopiperidin-3-yl)-1-oxoisoindolin-4-yl)oxy)acetyl)-2,6-diazaspiro[3.4]octan-2-yl)ethyl)-3-(3-((6-fluoronaphthalen-1-yl)oxy)propyl)-7-(1,3,5-trimethyl-1H-pyrazol-4-yl)-1H-indole-2-carboxylic acid (305)

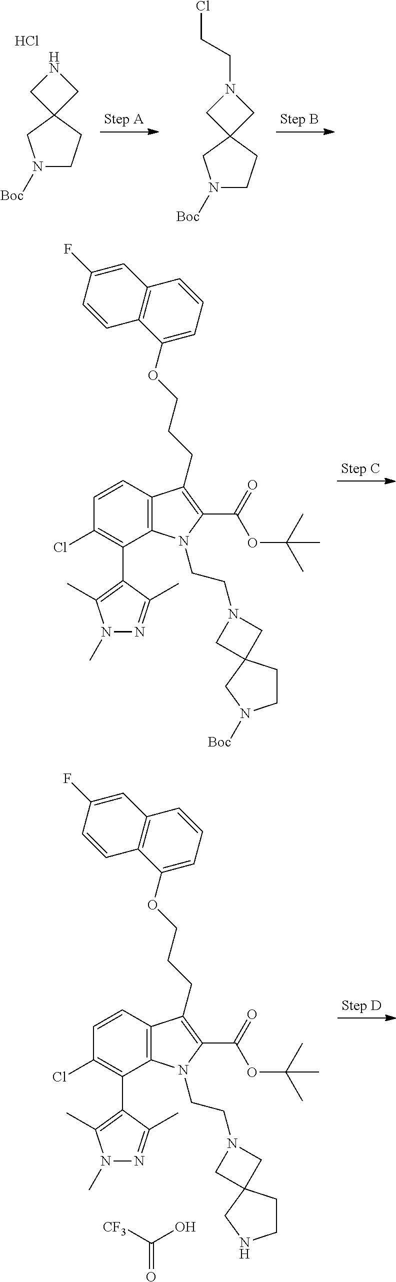

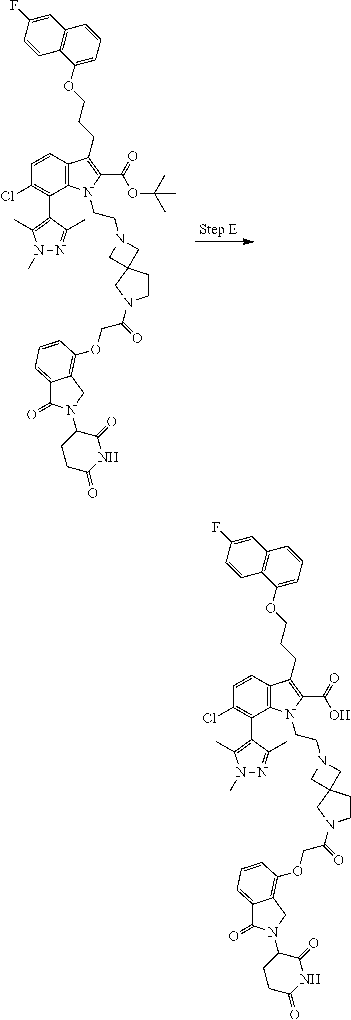 [SMILES: Cc1nn(C)c(C)c1-c1c(Cl)ccc2c(CCCOc3cccc4cc(F)ccc34)c(C(=O)OC(C)(C)C)n(CCN3CC4(CCN(C(=O)COc5cccc6c5CN(C5CCC(=O)NC5=O)C6=O)C4)C3)c12 ; Cc1nn(C)c(C)c1-c1c(Cl)ccc2c(CCCOc3cccc4cc(F)ccc34)c(C(=O)O)n(CCN3CC4(CCN(C(=O)COc5cccc6c5CN(C5CCC(=O)NC5=O)C6=O)C4)C3)c12]

### Step A

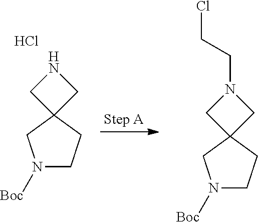 [SMILES: Cl ; CC(C)(C)OC(=O)N1CCC2(CNC2)C1 ; CC(C)(C)OC(=O)N1CCC2(CN(CCCl)C2)C1]

1-Bromo-2-chloroethane (400 mg, 2.80 mmol) and K₂CO₃ (960 mg, 6.95 mmol) were suspended in MeCN (2.8 mL). The suspension was heated to 70° C. under nitrogen atmosphere and tert-butyl 2,6-diazaspiro[3.4]octane-6-carboxylate hydrochloride (70.0 mg, 0.28 mmol) in DMF (1 mL) was added dropwise over 10 minutes. The reaction mixture was further stirred for 30 minutes at 70° C. After the reaction was cooled down to room temperature, the solid was filtered off, rinsed thoroughly with EtOAc and the volatiles were removed in vacuo. Purification by column chromatography (SiO₂, 0-10% MeOH in EtOAc) afforded tert-butyl 2-(2-chloroethyl)-2,6-diazaspiro[3.4]octane-6-carboxylate (63.0 mg, 0.23 mmol, 81%) as a colorless oil.

### Step B

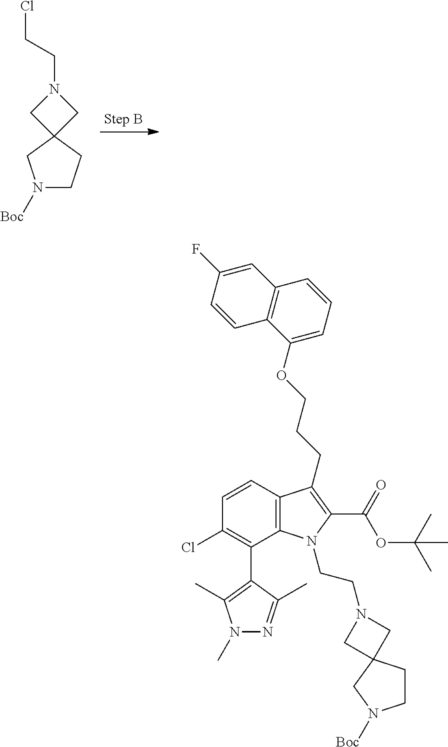 [SMILES: CC(C)(C)OC(=O)N1CCC2(CN(CCCl)C2)C1 ; Cc1nn(C)c(C)c1-c1c(Cl)ccc2c(CCCOc3cccc4cc(F)ccc34)c(C(=O)OC(C)(C)C)n(CCN3CC4(CCN(C(=O)OC(C)(C)C)C4)C3)c12]

tert-Butyl 6-chloro-3-(3-((6-fluoronaphthalen-1-yl)oxy)propyl)-7-(1,3,5-trimethyl-1H-pyrazol-4-yl)-1H-indole-2-carboxylate (100 mg, 0.178 mmol) and tert-butyl 2-(2-chloroethyl)-2,6-diazaspiro[3.4]octane-6-carboxylate (53.7 mg, 0.196 mmol) were dissolved in DMF (4 mL). Then, KI (29.5 mg, 178.0 μmol) was added followed by Cs₂CO₃ (174.0 mg, 0.534 mmol). The mixture was stirred at 70° C. for 12 hours. The reaction was quenched with water and aqueous layer was extracted once with EtOAc. Combined organics were washed three times with brine and water, dried over Na₂SO₄ and concentrated under vacuum. The crude was purified by column chromatography (SiO₂, 0-15% MeOH in EtOAc) to obtain tert-butyl 1-(2-(6-(tert-butoxycarbonyl)-2,6-diazaspiro[3.4]octan-2-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate (142 mg, 0.177 mmol, 99%) as a white solid.

LCMS (ESI+): m/z 800.1 [M+H]⁺

### Step C

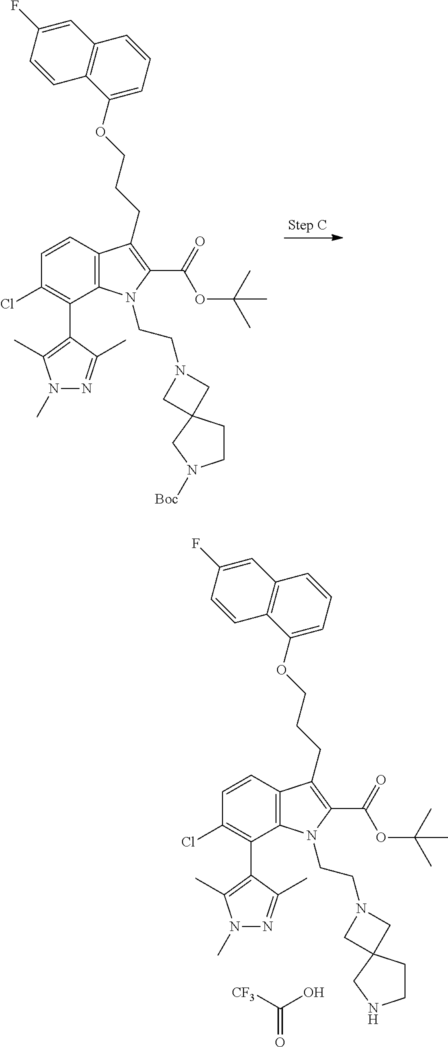

To a stirred solution of tert-butyl 1-(2-(6-(tert-butoxycarbonyl)-2,6-diazaspiro[3.4]octan-2-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate (100 mg, 0.125 mmol) in DCM (2.50 mL) at 0° C., TFA (0.144 mL) was added drop-wise and the resultant reaction mixture was stirred for 3 hours at room temperature. After complete consumption of the starting material, the reaction mixture was concentrated under reduced pressure. The crude residue was triturated with cyclohexane (3 times) and lyophilized to afford the desired compound 2-(2-(2-(tert-butoxycarbonyl)-6-chloro-3-(3-((6-fluoronaphthalen-1-yl)oxy)propyl)-7-(1,3,5-trimethyl-1H-pyrazol-4-yl)-1H-indol-1-yl)ethyl)-2,6-diazaspiro[3.4]octan-6-ium 2,2,2-trifluoroacetate (80 mg, 0.098 mmol, 78%) as an off white solid.

LCMS (ESI⁺): m/z 700.2 [M+H]⁺

### Step D

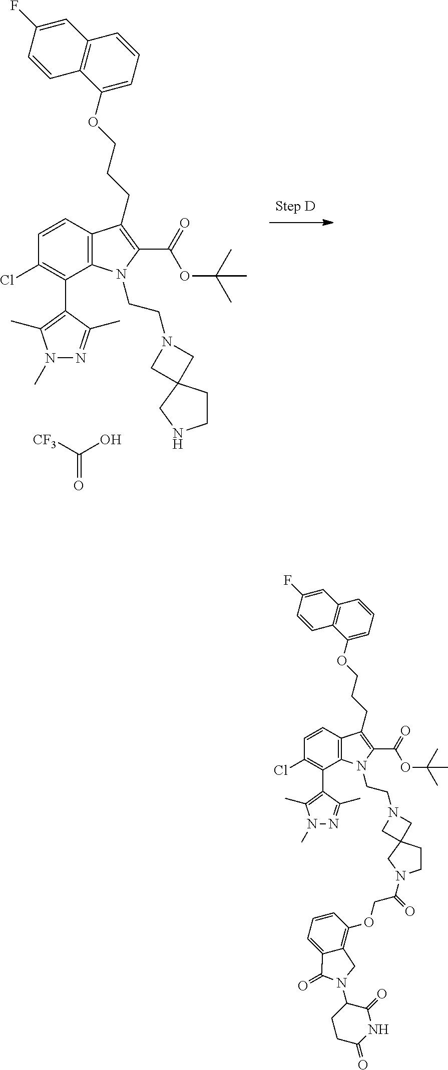 [SMILES: Cc1nn(C)c(C)c1-c1c(Cl)ccc2c(CCCOc3cccc4cc(F)ccc34)c(C(=O)OC(C)(C)C)n(CCN3CC4(CCNC4)C3)c12 ; O=C(O)C(F)(F)F ; Cc1nn(C)c(C)c1-c1c(Cl)ccc2c(CCCOc3cccc4cc(F)ccc34)c(C(=O)OC(C)(C)C)n(CCN3CC4(CCN(C(=O)COc5cccc6c5CN(C5CCC(=O)NC5=O)C6=O)C4)C3)c12]

To a solution of 2-((2-(2,6-dioxopiperidin-3-yl)-1-oxoisoindolin-4-yl)oxy)acetic acid (30.0 mg, 0.094 mmol) in DCM (1.9 mL) were added HATU (43.0 mg, 0.113 mmol) and 2-(2-(2-(tert-butoxycarbonyl)-6-chloro-3-(3-((6-fluoronaphthalen-1-yl)oxy)propyl)-7-(1,3,5-trimethyl-1H-pyrazol-4-yl)-1H-indol-1-yl)ethyl)-2,6-diazaspiro[3.4]octan-6-ium 2,2,2-trifluoroacetate (82.7 mg, 0.101 mmol) followed by the addition of DIPEA (0.084 mL, 0.471 mmol) and the reaction mixture was stirred for 16 h at room temperature. After complete consumption of the starting material, the reaction mixture was concentrated under reduced Then, the crude was purified by reverse phase column chromatography (C18, H₂O:MeCN+0.1% FA) to afford the desired product tert-butyl 6-chloro-1-(2-(6-(2-((2-(2,6-dioxopiperidin-3-yl)-1-oxoisoindolin-4-yl)oxy)acetyl)-2,6-diazaspiro[3.4]octan-2-yl)ethyl)-3-(3-((6-fluoronaphthalen-1-yl)oxy)propyl)-7-(1,3,5-trimethyl-1H-pyrazol-4-yl)-1H-indole-2-carboxylate (33.0 mg, 0.033 mmol, 35%) as a white solid.

LCMS (ESI+): m/z 1000 [M+H]⁺

### Step E

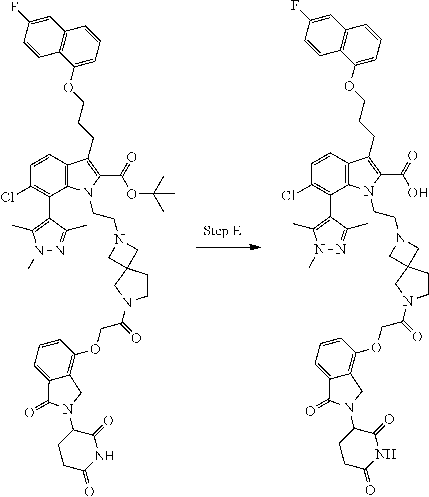 [SMILES: Cc1nn(C)c(C)c1-c1c(Cl)ccc2c(CCCOc3cccc4cc(F)ccc34)c(C(=O)OC(C)(C)C)n(CCN3CC4(CCN(C(=O)COc5cccc6c5CN(C5CCC(=O)NC5=O)C6=O)C4)C3)c12 ; Cc1nn(C)c(C)c1-c1c(Cl)ccc2c(CCCOc3cccc4cc(F)ccc34)c(C(=O)O)n(CCN3CC4(CCN(C(=O)COc5cccc6c5CN(C5CCC(=O)NC5=O)C6=O)C4)C3)c12]

To a stirred solution of tert-butyl 6-chloro-1-(2-(6-(2-((2-(2,6-dioxopiperidin-3-yl)-1-oxoisoindolin-4-yl)oxy)acetyl)-2,6-diazaspiro[3.4]octan-2-yl)ethyl)-3-(3-((6-fluoronaphthalen-1-yl)oxy)propyl)-7-(1,3,5-trimethyl-1H-pyrazol-4-yl)-1H-indole-2-carboxylate (30.0 mg, 0.030 mmol) in DCM (1.8 mL) at 0° C., TFA (1 mL) was added dropwise and the resultant reaction mixture was stirred for 4 days at room. After complete consumption of the starting material, the reaction mixture concentrated under reduced. Then the crude was purified by reverse phase column chromatography (C18, H₂O:MeCN+0.1% FA) to afford the desired product 6-chloro-1-(2-(6-(2-((2-(2,6-dioxopiperidin-3-yl)-1-oxoisoindolin-4-yl)oxy)acetyl)-2,6-diazaspiro[3.4]octan-2-yl)ethyl)-3-(3-((6-fluoronaphthalen-1-yl)oxy)propyl)-7-(1,3,5-trimethyl-1H-pyrazol-4-yl)-1H-indole-2-carboxylic acid (5.5 mg, 0.005 mmol, 19%) as a white solid.

LCMS (ESI⁺): m/z 944 [M+H]⁺

¹H NMR (400 MHz, DMSO) δ 10.98 (s, 1H), 8.23 (dd, J=9.3, 5.9 Hz, 1H), 7.69 (dd, J=8.5, 5.7 Hz, 1H), 7.64 (dt, J=10.4, 2.4 Hz, 1H), 7.48-7.31 (m, 4H), 7.30 (d, J=7.4 Hz, 1H), 7.21 (dd, J=8.5, 2.5 Hz, 1H), 7.09 (dd, J=8.2, 3.0 Hz, 1H), 6.93-6.78 (m, 1H), 5.10 (dd, J=13.2, 5.1 Hz, 1H), 4.81 (d, J=7.6 Hz, 2H), 4.36 (dd, J=17.4, 2.6 Hz, 1H), 4.28-3.97 (m, 5H), 3.75 (s, 3H), 3.26-3.14 (m, 6H), 3.13-2.96 (m, 2H), 2.98-2.84 (m, 1H), 2.62-2.57 (m, 1H), 2.43-2.38 (m, 1H), 2.25-2.13 (m, 2H), 2.10-1.93 (m, 5H), 1.93-1.81 (m, 4H).

4 protons in aliphatic area overlap with water.

### Example 75. 6-Chloro-1-{2-[5-(2-{[2-(2,6-dioxopiperidin-3-yl)-1-oxo-2,3-dihydro-1H-isoindol-4-yl]oxy}acetamido)-1-methylpiperidin-2-yl]ethyl}-3-{3-[(6-fluoronaphthalen-1-yl)oxy]propyl}-7-(1,3,5-trimethyl-1H-pyrazol-4-yl)-1H-indole-2-carboxylic acid (306)

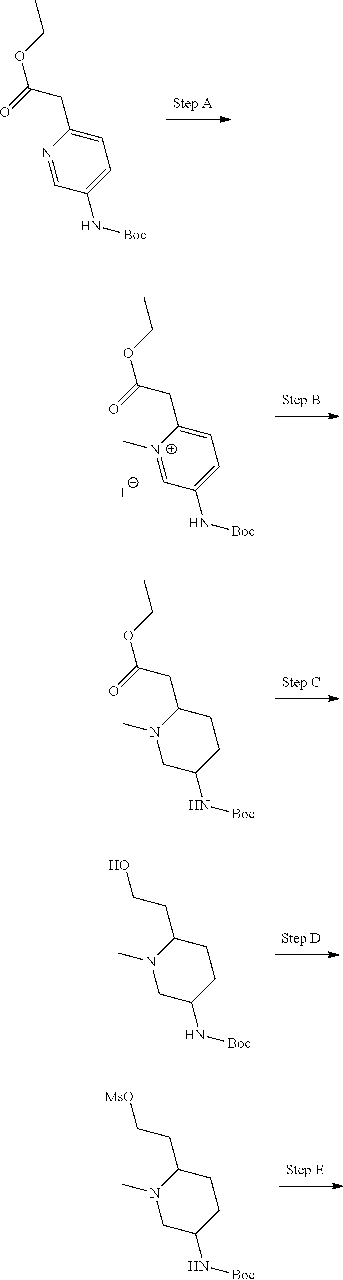 [SMILES: CCOC(=O)Cc1ccc(NC(=O)OC(C)(C)C)cn1 ; CCOC(=O)Cc1ccc(NC(=O)OC(C)(C)C)c[n+]1C ; [I-] ; CCOC(=O)CC1CCC(NC(=O)OC(C)(C)C)CN1C ; CN1CC(NC(=O)OC(C)(C)C)CCC1CCO ; CN1CC(NC(=O)OC(C)(C)C)CCC1CCOS(C)(=O)=O]

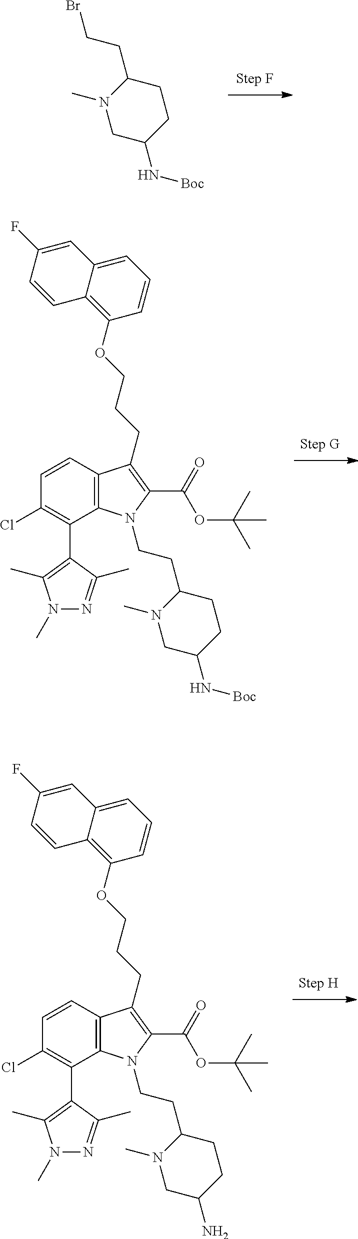

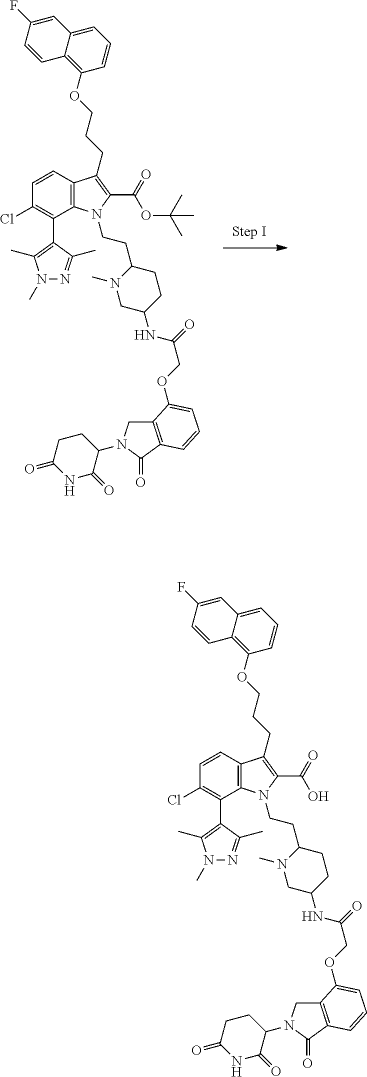 [SMILES: Cc1nn(C)c(C)c1-c1c(Cl)ccc2c(CCCOc3cccc4cc(F)ccc34)c(C(=O)OC(C)(C)C)n(CCC3CCC(NC(=O)COc4cccc5c4CN(C4CCC(=O)NC4=O)C5=O)CN3C)c12 ; Cc1nn(C)c(C)c1-c1c(Cl)ccc2c(CCCOc3cccc4cc(F)ccc34)c(C(=O)O)n(CCC3CCC(NC(=O)COc4cccc5c4CN(C4CCC(=O)NC4=O)C5=O)CN3C)c12]

### Step A

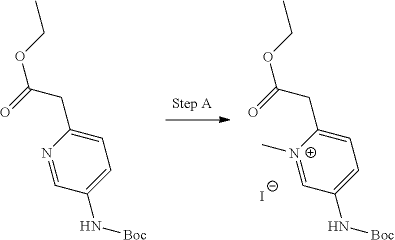 [SMILES: CCOC(=O)Cc1ccc(NC(=O)OC(C)(C)C)cn1 ; CCOC(=O)Cc1ccc(NC(=O)OC(C)(C)C)c[n+]1C ; [I-]]

To a solution of ethyl 2-(5-((tert-butoxycarbonyl)amino)pyridin-2-yl)acetate (23.0 g, 82.0 mmol) in THE (200 mL) was added methyl iodide (60.0 mL, 820.4 mmol). The reaction mixture was allowed to stir at room temperature for 16 h. After complete consumption of the starting material, reaction mixture was diluted thoroughly with Et₂O and pentane solvent. The residues were dried under vacuum to get of 5-((tert-butoxycarbonyl)amino)-2-(2-ethoxy-2-oxoethyl)-1-methylpyridin-1-ium iodide (27.0 g, 63.9 mmol, 78%) as pale yellow solid.

LCMS (ESI+): m/z 294.9 [M+H]⁺

### Step B

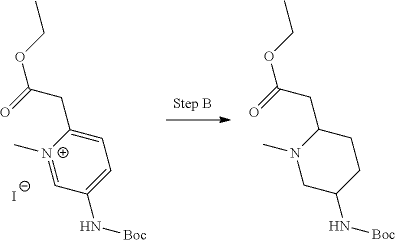 [SMILES: CCOC(=O)Cc1ccc(NC(=O)OC(C)(C)C)c[n+]1C ; [I-] ; CCOC(=O)CC1CCC(NC(=O)OC(C)(C)C)CN1C]

A well stirred solution of 5-((tert-butoxycarbonyl)amino)-2-(2-ethoxy-2-oxoethyl)-1-methylpyridin-1-ium iodide (27.0 g, 63.9 mmol) in EtOH (500 mL) was deoxygenated using nitrogen for 15 minutes in a 1 L autoclave vessel. After that, PtO₂ (6.235 g, 27.5 mmol) was added and the reaction vessel was backfilled with hydrogen. The reaction mixture was then allowed to stir for 72 h at 70° C. under 80 PSI pressure. After complete consumption of the starting material, the reaction mixture was filtered through sintered funnel through Celite®® pad and filtrate was concentrated to get crude 20.0 g of ethyl 2-(5-((tert-butoxycarbonyl)amino)-1-methylpiperidin-2-yl)acetate as reddish brown sticky solid.

LCMS (ESI+): m/z 301.1 [M+H]⁺

### Step C

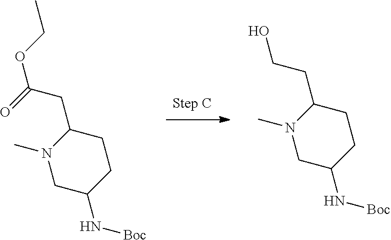 [SMILES: CCOC(=O)CC1CCC(NC(=O)OC(C)(C)C)CN1C ; CN1CC(NC(=O)OC(C)(C)C)CCC1CCO]

To a stirred solution of ethyl 2-(5-((tert-butoxycarbonyl)amino)-1-methylpiperidin-2-yl)acetate (6.5 g, crude) in dry THE (120 mL) was added LiAlH₄ (1 M in THF; 45.0 mL) dropwise at 0° C. for 15 minutes under nitrogen. Reaction mixture was allowed to stir at the same temperature for 8 h under nitrogen. After complete consumption of the starting material, the reaction mixture was quenched with saturated Na₂SO₄ solution. The reaction mixture was filtered and the filtrate was evaporated to get crude product which was purified by reverse phase preparative HPLC (C18, 20 mM ammonium bicarbonate in H₂O:MeCN) to get tert-butyl (6-(2-hydroxyethyl)-1-methylpiperidin-3-yl)carbamate (1.0 g, 3.87 mmol, 18.7% over 2 steps) as yellow solid.

LCMS (ESI+): m/z 259.0 [M+H]⁺

### Step D

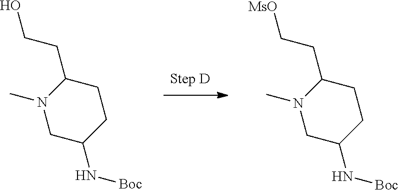 [SMILES: CN1CC(NC(=O)OC(C)(C)C)CCC1CCO ; CN1CC(NC(=O)OC(C)(C)C)CCC1CCOS(C)(=O)=O]

To a well stirred solution of tert-butyl (6-(2-hydroxyethyl)-1-methylpiperidin-3-yl)carbamate (1.0 g, 3.87 mmol) in DCM (30 mL) was added MsCl (0.3 mL, 3.871 mmol) and Et₃N (0.65 mL, 4.64 mmol) at 0° C. under nitrogen. The reaction mixture was then allowed to stir at ambient temperature for 2 h. After complete consumption of the starting material, the reaction mixture was evaporated under reduced pressure to get 1.3 g of crude 2-(5-((tert-butoxycarbonyl)amino)-1-methylpiperidin-2-yl)ethyl methanesulfonate which was used in the next step without further purification.

### Step E

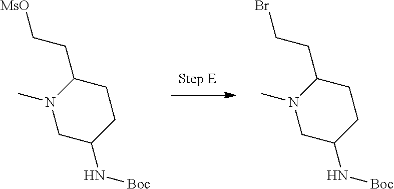 [SMILES: CN1CC(NC(=O)OC(C)(C)C)CCC1CCOS(C)(=O)=O ; CN1CC(NC(=O)OC(C)(C)C)CCC1CCBr]

To a stirred solution of 2-(5-((tert-butoxycarbonyl)amino)-1-methylpiperidin-2-yl)ethyl methanesulfonate (1.3 g, crude) in THE (50.0 mL) was added LiBr (1.007 g, 11.592 mmol) at room temperature under nitrogen. The reaction mixture was allowed to stir at 70° C. for 6 h. After complete consumption of the starting material, the reaction mixture was concentrated, diluted with water and extracted with 5% MeOH in DCM solvent system. After that, the organic layer was dried over anhydrous Na₂SO₄ and evaporated under reduced pressure to get 400 mg of crude tert-butyl (6-(2-bromoethyl)-1-methylpiperidin-3-yl)carbamate as reddish brown sticky liquid which was used in the next step without further purification.

### Step F

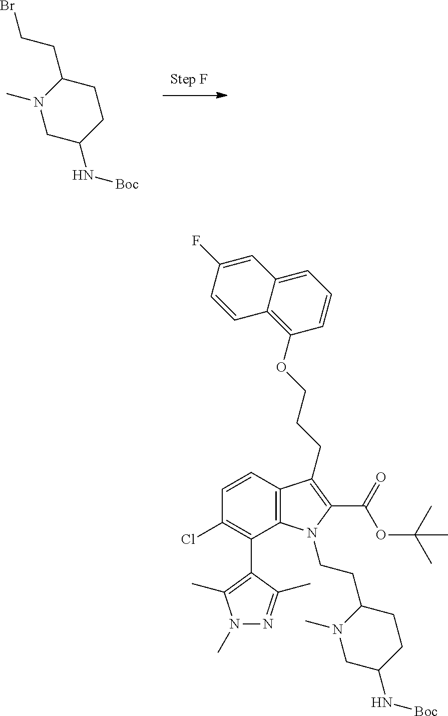 [SMILES: CN1CC(NC(=O)OC(C)(C)C)CCC1CCBr ; Cc1nn(C)c(C)c1-c1c(Cl)ccc2c(CCCOc3cccc4cc(F)ccc34)c(C(=O)OC(C)(C)C)n(CCC3CCC(NC(=O)OC(C)(C)C)CN3C)c12]

To a well stirred solution of tert-butyl 6-chloro-3-(3-((6-fluoronaphthalen-1-yl)oxy)propyl)-7-(1,3,5-trimethyl-1H-pyrazol-4-yl)-1H-indole-2-carboxylate (230.0 mg, 0.41 mmol) in DMF (10.0 mL) was added 400 mg crude of tert-butyl (6-(2-bromoethyl)-1-methylpiperidin-3-yl)carbamate followed by Cs₂CO₃ (300 mg, 0.82 mmol) and KI (68.06 mg, 0.41 mmol) and the mixture was allowed to stir at 90° C. for 16 h under nitrogen. After complete consumption of the starting material, the reaction mixture was diluted with EtOAc, washed thoroughly with cold water followed and brine. Then the organic layer was evaporated to dryness to get crude which was purified by column chromatography (SiO₂, 30% EtOAc in DCM) to afford tert-butyl 1-(2-(5-((tert-butoxycarbonyl)amino)-1-methylpiperidin-2-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate (300 mg, 0.374 mmol, 91%) as white solid.

LCMS (ESI+): m/z 802.5 [M+H]⁺

### Step G

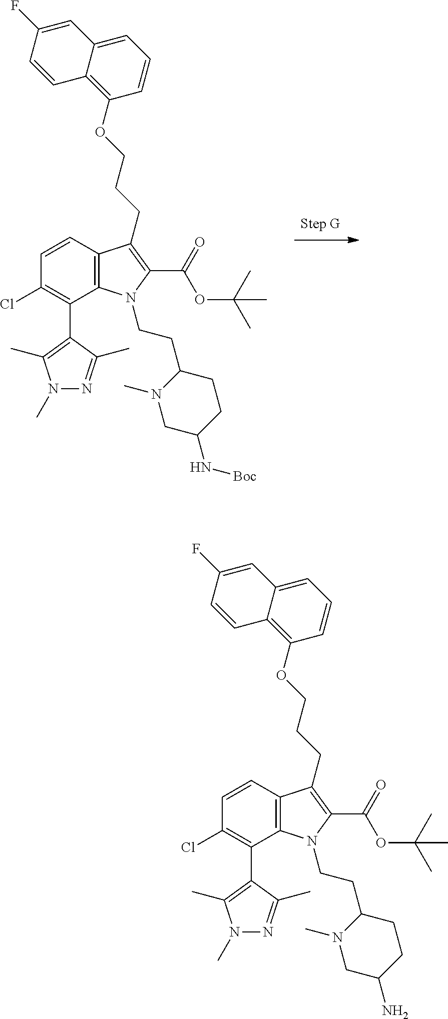 [SMILES: Cc1nn(C)c(C)c1-c1c(Cl)ccc2c(CCCOc3cccc4cc(F)ccc34)c(C(=O)OC(C)(C)C)n(CCC3CCC(NC(=O)OC(C)(C)C)CN3C)c12 ; Cc1nn(C)c(C)c1-c1c(Cl)ccc2c(CCCOc3cccc4cc(F)ccc34)c(C(=O)OC(C)(C)C)n(CCC3CCC(N)CN3C)c12]

To a stirred solution of tert-butyl 1-(2-(5-((tert-butoxycarbonyl)amino)-1-methylpiperidin-2-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate (300.0 mg, 0.374 mmol) in DCM (30.0 mL) was added 4M dioxane in HCl (6.0 mL) at 0° C. dropwise to the reaction mixture which was allowed to stir at 0° C. for 4 h under nitrogen. After complete consumption of the starting material, the reaction mixture was poured in to cold 1M NaOH solution and extracted several times with DCM. The combined organics were dried over anhydrous Na₂SO₄ and concentrated in vacuo to get the crude compound which was then purified by column chromatography (amine silica, 2% MeOH in DCM) to get tert-butyl 1-(2-(5-amino-1-methylpiperidin-2-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate (200.0 mg, 0.285 mmol, 76%) of as off white solid.

LCMS (ESI+): m/z 702.5 [M+H]⁺

### Step H

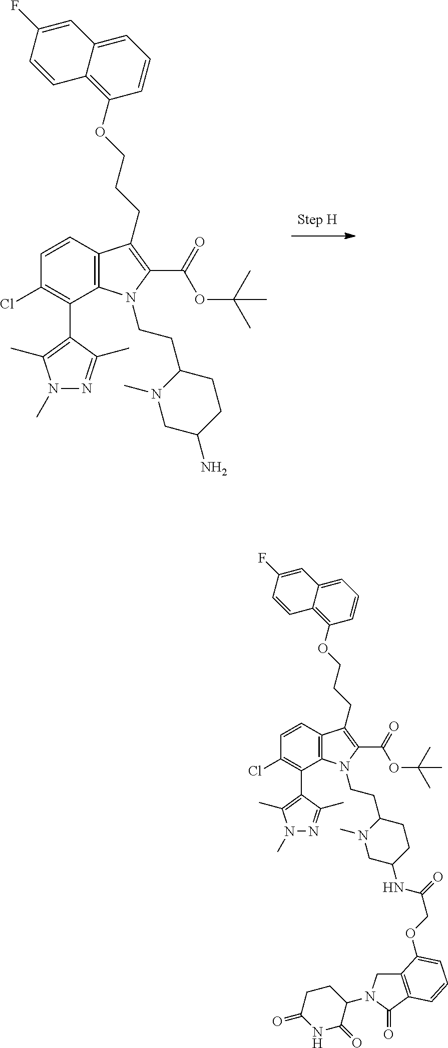 [SMILES: Cc1nn(C)c(C)c1-c1c(Cl)ccc2c(CCCOc3cccc4cc(F)ccc34)c(C(=O)OC(C)(C)C)n(CCC3CCC(N)CN3C)c12 ; Cc1nn(C)c(C)c1-c1c(Cl)ccc2c(CCCOc3cccc4cc(F)ccc34)c(C(=O)OC(C)(C)C)n(CCC3CCC(NC(=O)COc4cccc5c4CN(C4CCC(=O)NC4=O)C5=O)CN3C)c12]

2-{[2-(2,6-Dioxopiperidin-3-yl)-1-oxo-2,3-dihydro-1H-isoindol-4-yl]oxy}acetic acid (13.6 mg, 0.043 mmol) and HATU (14.9 mg, 0.039 mmol) were dissolved in dry DMF (1.0 mL) under argon atmosphere and DIPEA (0.019 mL, 0.107 mmol) was added. Reaction was stirred for 10 min at room temperature and to it tert-butyl 1-[2-(5-amino-1-methylpiperidin-2-yl)ethyl]-6-chloro-3-{3-[(6-fluoronaphthalen-1-yl)oxy]propyl}-7-(1,3,5-trimethyl-1H-pyrazol-4-yl)-1H-indole-2-carboxylate (25.0 mg, 0.036 mmol) was added as a solution in DMF (0.5 mL). Reaction was stirred at room temperature under argon atmosphere. After 1 h additional portion of 2-{[2-(2,6-dioxopiperidin-3-yl)-1-oxo-2,3-dihydro-1H-isoindol-4-yl]oxy}acetic acid (6.8 mg, 0.021 mmol), HATU (7.4 mg, 0.020 mmol) and DIPEA (0.009 mL, 0.053 mmol) were mixed alongside in a separate vial for 10 min in 0.5 mL of DMF at room temperature. Then, the solutions were combined and the reaction was continued for 30 min in 45° C. After complete consumption of the starting material, the solution was diluted with DCM and washed with brine and water. The organic layer was dried with anhydrous MgSO₄, filtered, concentrated and dried under reduced pressure. The crude tert-butyl 6-chloro-1-{2-[5-(2-{[2-(2,6-dioxopiperidin-3-yl)-1-oxo-2,3-dihydro-1H-isoindol-4-yl]oxy}acetamido)-1-methylpiperidin-2-yl]ethyl}-3-{3-[(6-fluoronaphthalen-1-yl)oxy]propyl}-7-(1,3,5-trimethyl-1H-pyrazol-4-yl)-1H-indole-2-carboxylate (41.3 mg) was obtained as yellow oil and was used in the next step without further purification.

LCMS (ESI+): m/z 1002.7 [M+H]⁺

### Step I

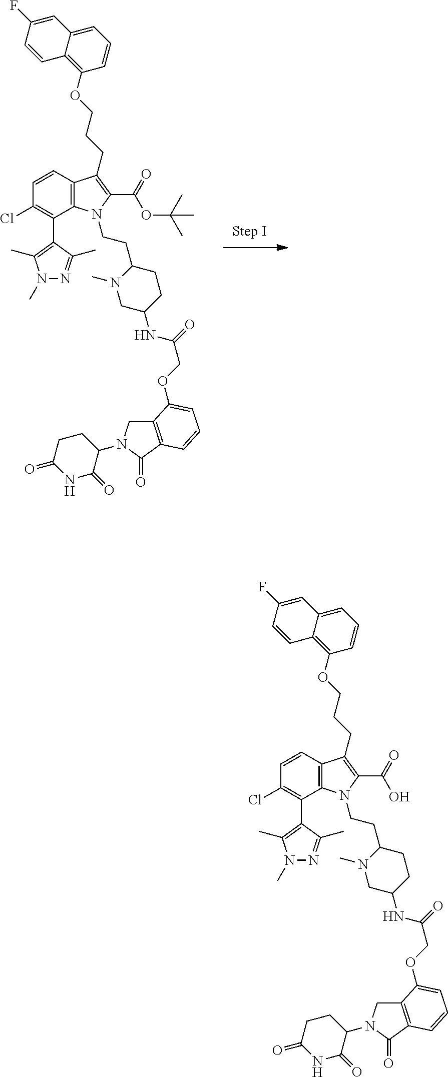 [SMILES: Cc1nn(C)c(C)c1-c1c(Cl)ccc2c(CCCOc3cccc4cc(F)ccc34)c(C(=O)OC(C)(C)C)n(CCC3CCC(NC(=O)COc4cccc5c4CN(C4CCC(=O)NC4=O)C5=O)CN3C)c12 ; Cc1nn(C)c(C)c1-c1c(Cl)ccc2c(CCCOc3cccc4cc(F)ccc34)c(C(=O)O)n(CCC3CCC(NC(=O)COc4cccc5c4CN(C4CCC(=O)NC4=O)C5=O)CN3C)c12]

Crude tert-butyl 6-chloro-1-{2-[5-(2-{[2-(2,6-dioxopiperidin-3-yl)-1-oxo-2,3-dihydro-1H-isoindol-4-yl]oxy}acetamido)-1-methylpiperidin-2-yl]ethyl}-3-{3-[(6-fluoronaphthalen-1-yl)oxy]propyl}-7-(1,3,5-trimethyl-1H-pyrazol-4-yl)-1H-indole-2-carboxylate (41.3 mg, 0.041 mmol) was dissolved in dry DCM (0.315 mL) under argon atmosphere and TFA (0.315 mL, 4.119 mmol) was added. Reaction was stirred for 16 h at room temperature under argon atmosphere. After complete consumption of the starting material, the mixture was concentrated and dried under reduced pressure. The resulting residue was dissolved in DMSO and purified with preparative HPLC (C18, H₂O:MeCN+0.1% FA) to give 6-chloro-1-{2-[5-(2-{[2-(2,6-dioxopiperidin-3-yl)-1-oxo-2,3-dihydro-1H-isoindol-4-yl]oxy}acetamido)-1-methylpiperidin-2-yl]ethyl}-3-{3-[(6-fluoronaphthalen-1-yl)oxy]propyl}-7-(1,3,5-trimethyl-1H-pyrazol-4-yl)-1H-indole-2-carboxylic acid (11.7 mg, 0.012 mmol, 33% over 2 steps) as white solid.

LCMS (ESI+): m/z 946.2 [M+H]⁺

¹H NMR (500 MHz, DMSO, 353 K) δ 10.62 (s, 1H), 8.27 (ddd, J=9.2, 5.9, 1.5 Hz, 1H), 7.71 (dt, J=8.5, 1.4 Hz, 1H), 7.60 (dd, J=10.4, 2.6 Hz, 1H), 7.54-7.42 (m, 4H), 7.40-7.32 (m, 2H), 7.26-7.18 (m, 2H), 6.90 (dd, J=5.9, 2.8 Hz, 1H), 5.07-4.99 (m, 1H), 4.80-4.62 (m, 2H), 4.53-4.33 (m, 2H), 4.26 (t, J=6.2 Hz, 2H), 4.24-4.07 (m, 2H), 3.91-3.82 (m, 1H), 3.74 (dd, J=10.4, 3.0 Hz, 3H), 3.29 (t, J=7.5 Hz, 2H), 2.89-2.81 (m, 1H), 2.63-2.54 (m, 1H), 2.50-2.32 (m, 2H), 2.31-2.20 (m, 3H), 2.07 (dd, J=7.4, 3.7 Hz, 3H), 2.03 (s, 2H), 1.99 (d, J=6.3 Hz, 2H), 1.88 (dd, J=23.7, 3.6 Hz, 3H), 1.83-1.74 (m, 1H), 1.61-1.49 (m, 2H), 1.49-1.34 (m, 2H), 1.28-1.23 (m, 1H), 1.15-1.03 (m, 1H)

### Example 76. 6-chloro-1-(2-(5-((2-(2,6-dioxopiperidin-3-yl)-1,3-dioxoisoindolin-4-yl)amino)-1-methylpiperidin-2-yl)ethyl)-3-(3-((6-fluoronaphthalen-1-yl)oxy)propyl)-7-(1,3,5-trimethyl-1H-pyrazol-4-yl)-1H-indole-2-carboxylic acid (307)

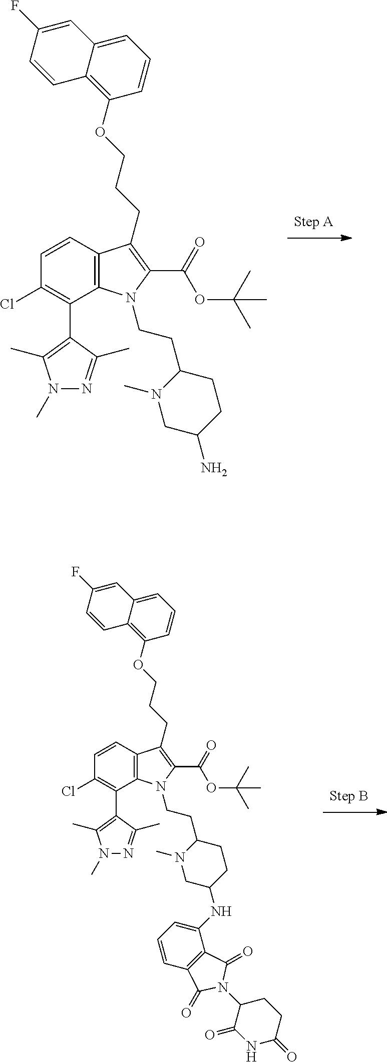

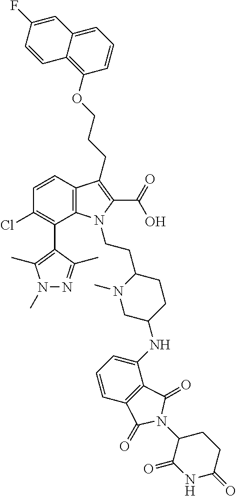 [SMILES: Cc1nn(C)c(C)c1-c1c(Cl)ccc2c(CCCOc3cccc4cc(F)ccc34)c(C(=O)O)n(CCC3CCC(Nc4cccc5c4C(=O)N(C4CCC(=O)NC4=O)C5=O)CN3C)c12]

### Step A

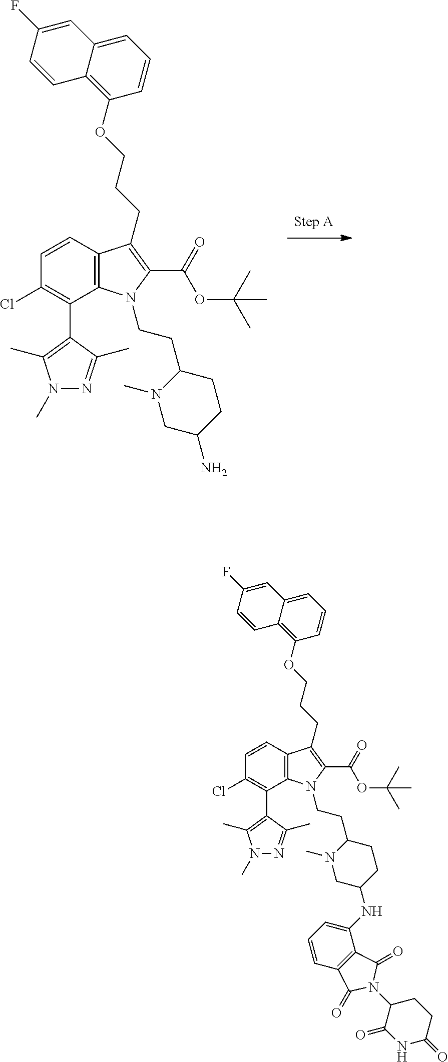 [SMILES: Cc1nn(C)c(C)c1-c1c(Cl)ccc2c(CCCOc3cccc4cc(F)ccc34)c(C(=O)OC(C)(C)C)n(CCC3CCC(N)CN3C)c12 ; Cc1nn(C)c(C)c1-c1c(Cl)ccc2c(CCCOc3cccc4cc(F)ccc34)c(C(=O)OC(C)(C)C)n(CCC3CCC(Nc4cccc5c4C(=O)N(C4CCC(=O)NC4=O)C5=O)CN3C)c12]

tert-Butyl 1-[2-(5-amino-1-methylpiperidin-2-yl)ethyl]-6-chloro-3-{3-[(6-fluoronaphthalen-1-yl)oxy]propyl}-7-(1,3,5-trimethyl-1H-pyrazol-4-yl)-1H-indole-2-carboxylate (15.0 mg, 0.021 mmol) and 2-(2,6-dioxopiperidin-3-yl)-4-fluoro-2,3-dihydro-1H-isoindole-1,3-dione (8.8 mg, 0.032 mmol) were dissolved in dry DMSO (1.1 mL) and DIPEA (0.019 mL, 0.107 mmol) was added. The reaction was stirred at 90° C. for 20 h. After complete consumption of the starting material, the solution was cooled down to room temperature and purified with preparative HPLC (C18, H₂O:MeCN+0.1% FA) to give tert-butyl 6-chloro-1-[2-(5-{[2-(2,6-dioxopiperidin-3-yl)-1,3-dioxo-2,3-dihydro-1H-isoindol-4-yl]amino}-1-methylpiperidin-2-yl)ethyl]-3-{3-[(6-fluoronaphthalen-1-yl)oxy]propyl}-7-(1,3,5-trimethyl-1H-pyrazol-4-yl)-1H-indole-2-carboxylate (11.2 mg, 0.012 mmol, 54.7%) as yellow solid.

LCMS (ESI+): m/z 959 [M+H]⁺

### Step B

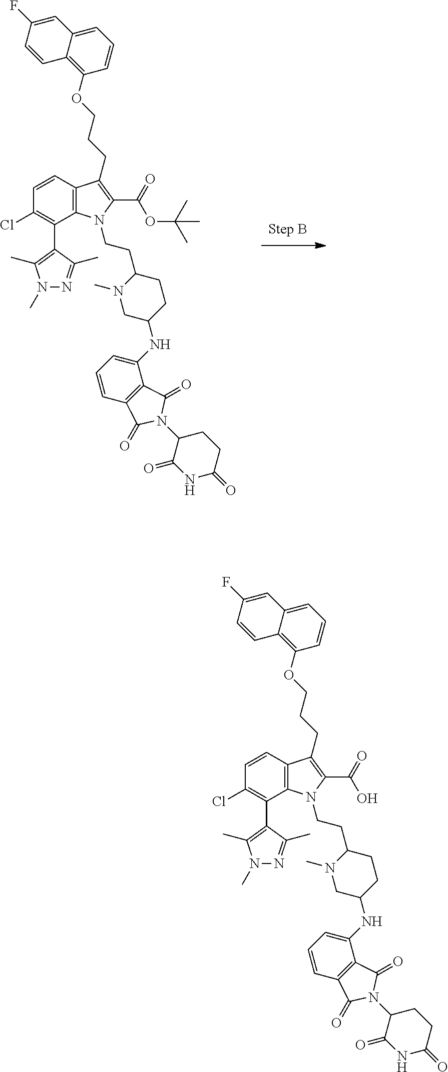 [SMILES: Cc1nn(C)c(C)c1-c1c(Cl)ccc2c(CCCOc3cccc4cc(F)ccc34)c(C(=O)OC(C)(C)C)n(CCC3CCC(Nc4cccc5c4C(=O)N(C4CCC(=O)NC4=O)C5=O)CN3C)c12 ; Cc1nn(C)c(C)c1-c1c(Cl)ccc2c(CCCOc3cccc4cc(F)ccc34)c(C(=O)O)n(CCC3CCC(Nc4cccc5c4C(=O)N(C4CCC(=O)NC4=O)C5=O)CN3C)c12]

tert-Butyl 6-chloro-1-[2-(5-{[2-(2,6-dioxopiperidin-3-yl)-1,3-dioxo-2,3-dihydro-1H-isoindol-4-yl]amino}-1-methylpiperidin-2-yl)ethyl]-3-{3-[(6-fluoronaphthalen-1-yl)oxy]propyl}-7-(1,3,5-trimethyl-1H-pyrazol-4-yl)-1H-indole-2-carboxylate (10.7 mg, 0.011 mmol) was dissolved in FA (2.1 mL, 50.076 mmol) and the reaction was stirred in a sealed vial for 72 h at room temperature and then at 60° C. for 4 h. After consumption of the starting material, reaction was concentrated under reduced pressure, suspended in water and dry-freezed to give 6-chloro-1-[2-(5-{[2-(2,6-dioxopiperidin-3-yl)-1,3-dioxo-2,3-dihydro-1H-isoindol-4-yl]amino}-1-methylpiperidin-2-yl)ethyl]-3-{3-[(6-fluoronaphthalen-1-yl)oxy]propyl}-7-(1,3,5-trimethyl-1H-pyrazol-4-yl)-1H-indole-2-carboxylic acid (8.1 mg, 0.009 mmol, 80.4%) as yellow solid.

LCMS (ESI+): m/z 902.2 [M+H]⁺

¹H NMR (500 MHz, DMSO, 353 K) δ 10.88-10.65 (m, 1H), 8.30-8.20 (m, 1H), 7.76-7.66 (m, 1H), 7.62-7.54 (m, 2H), 7.46-7.39 (m, 2H), 7.37-7.29 (m, 1H), 7.24-7.18 (m, 1H), 7.11-6.99 (m, 2H), 6.91-6.85 (m, 1H), 6.84-6.72 (m, 1H), 5.07-4.96 (m, 1H), 4.37-4.09 (m, 4H), 3.88-3.73 (m, 1H), 3.66-3.47 (m, 3H), 3.34-3.24 (m, 2H), 2.89-2.80 (m, 1H), 2.75-2.54 (m, 3H), 2.36-2.28 (m, 1H), 2.28-2.17 (m, 2H), 2.11 (d, J=10.2 Hz, 3H), 2.08-2.02 (m, 1H), 2.02-1.96 (m, 3H), 1.86 (d, J=2.3 Hz, 3H), 1.76-1.64 (m, 1H), 1.63-1.35 (m, 3H), 1.23-1.02 (m, 2H), 0.99-0.88 (m, 1H).

### Example 77. 6-Chloro-1-(2-(4-((2-(2,6-dioxopiperidin-3-yl)-1,3-dioxoisoindolin-4-yl)amino)piperidin-1-yl)ethyl)-3-(3-((6-fluoronaphthalen-1-yl)oxy)propyl)-7-(1,3,5-trimethyl-1H-pyrazol-4-yl)-3a,7a-dihydro-1H-indole-2-carboxylic acid (308)

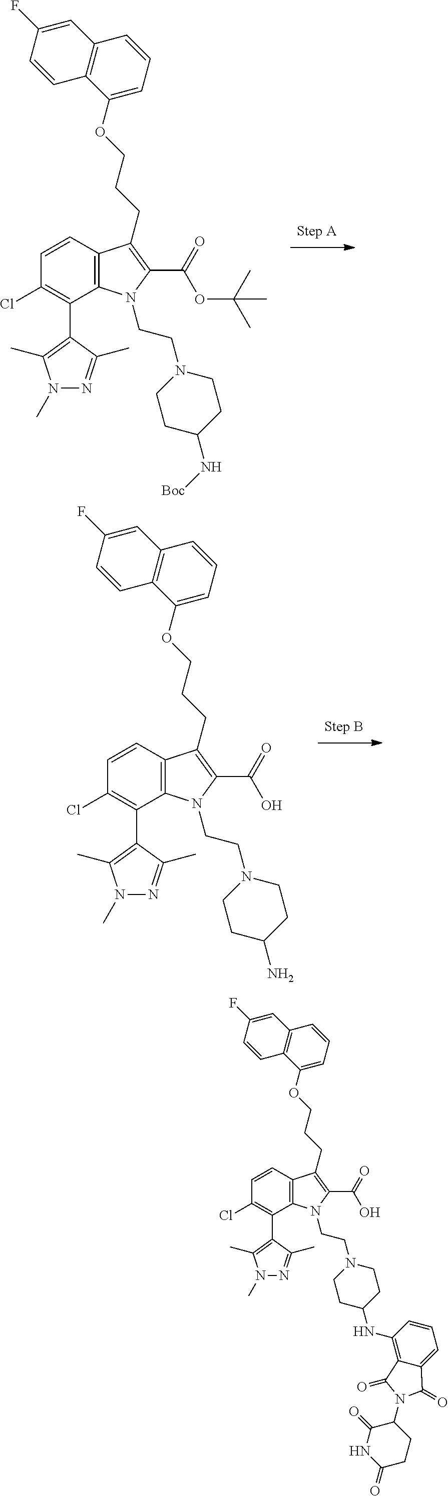

### Step A

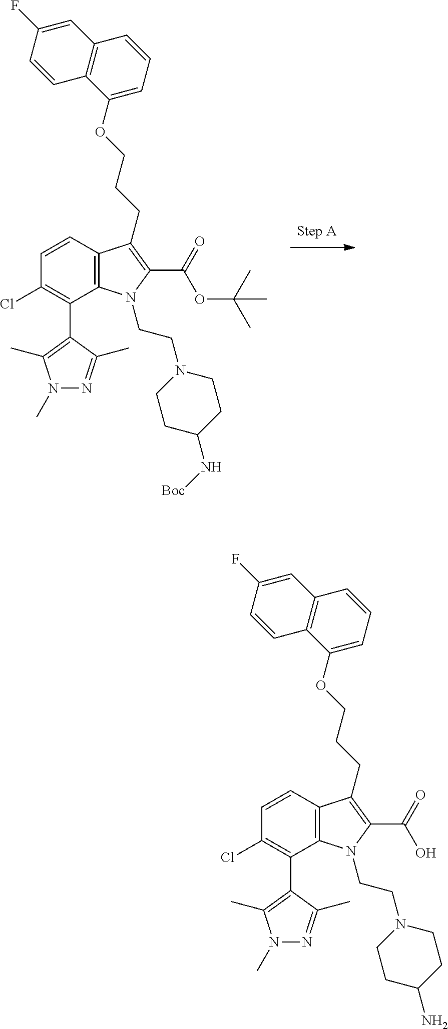 [SMILES: Cc1nn(C)c(C)c1-c1c(Cl)ccc2c(CCCOc3cccc4cc(F)ccc34)c(C(=O)OC(C)(C)C)n(CCN3CCC(NC(=O)OC(C)(C)C)CC3)c12 ; Cc1nn(C)c(C)c1-c1c(Cl)ccc2c(CCCOc3cccc4cc(F)ccc34)c(C(=O)O)n(CCN3CCC(N)CC3)c12]

To a solution of tert-butyl 1-[2-(4-{[(tert-butoxy)carbonyl]amino}piperidin-1-yl)ethyl]-6-chloro-3-{3-[(6-fluoronaphthalen-1-yl)oxy]propyl}-7-(1,3,5-trimethyl-1H-pyrazol-4-yl)-1H-indole-2-carboxylate (45.0 mg, 0.058 mmol) in DCM (1.0 mL) was added TFA (1.0 mL, 13.059 mmol). The mixture was stirred for 18 h at RT. The crude was concentrated in vacuo and purified by reverse phase flash chromatography (C18, H₂O:MeCN+0.1% FA) to obtain 1-[2-(4-aminopiperidin-1-yl)ethyl]-6-chloro-3-{3-[(6-fluoronaphthalen-1-yl)oxy]propyl}-7-(1,3,5-trimethyl-1H-pyrazol-4-yl)-1H-indole-2-carboxylic acid (33.0 mg, 0.052 mmol, 89%) as a white solid.

LCMS (ESI+): m/z 632.25 [M+H]⁺

### Step B

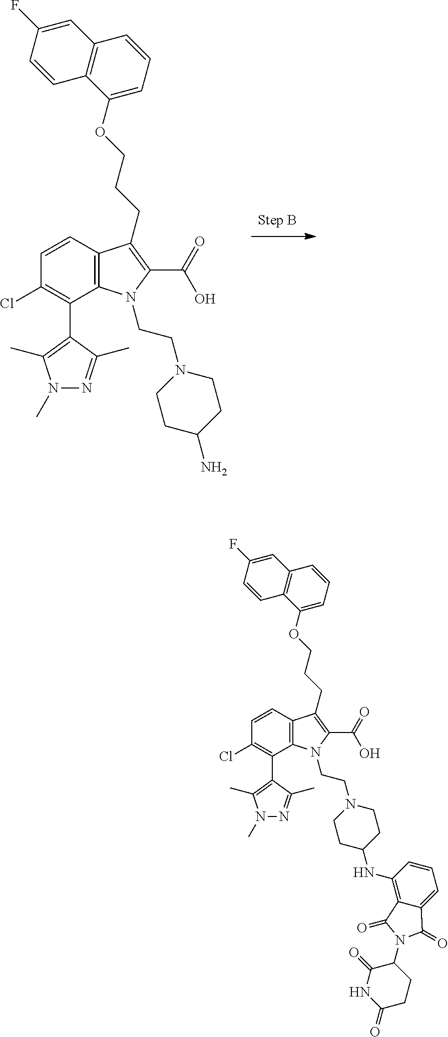 [SMILES: Cc1nn(C)c(C)c1-c1c(Cl)ccc2c(CCCOc3cccc4cc(F)ccc34)c(C(=O)O)n(CCN3CCC(N)CC3)c12 ; Cc1nn(C)c(C)c1-c1c(Cl)ccc2c(CCCOc3cccc4cc(F)ccc34)c(C(=O)O)n(CCN3CCC(Nc4cccc5c4C(=O)N(C4CCC(=O)NC4=O)C5=O)CC3)c12]

To a solution of 1-[2-(4-aminopiperidin-1-yl)ethyl]-6-chloro-3-{3-[(6-fluoronaphthalen-1-yl)oxy]propyl}-7-(1,3,5-trimethyl-1H-pyrazol-4-yl)-1H-indole-2-carboxylic acid (33.0 mg, 0.052 mmol) and 2-(2,6-dioxopiperidin-3-yl)-4-fluoro-2,3-dihydro-1H-isoindole-1,3-dione (21.6 mg, 0.078 mmol) in DMSO (1.0 mL) was added DIPEA (0.045 mL, 0.261 mmol). The mixture was stirred for 18 h at 90° C. The crude was purified by preparative HPLC (C18, H₂O:MeCN+0.1% FA) to obtain 6-chloro-1-[2-(4-{[2-(2,6-dioxopiperidin-3-yl)-1,3-dioxo-2,3-dihydro-1H-isoindol-4-yl]amino}piperidin-1-yl)ethyl]-3-{3-[(6-fluoronaphthalen-1-yl)oxy]propyl}-7-(1,3,5-trimethyl-1H-pyrazol-4-yl)-1H-indole-2-carboxylic acid (5.8 mg, 0.007 mmol, 12.5%) as a yellow solid.

LCMS (ESI+): m/z 888.25 [M+H]⁺

¹H NMR (500 MHz, DMSO, 353 K) δ 10.76 (s, 1H), 8.24 (dd, J=9.2, 5.9 Hz, 1H), 7.66 (d, J=8.5 Hz, 1H), 7.60-7.52 (m, 2H), 7.44-7.36 (m, 2H), 7.31 (td, J=8.9, 2.7 Hz, 1H), 7.19 (d, J=8.5 Hz, 1H), 7.07 (d, J=8.6 Hz, 1H), 7.05-7.00 (m, 1H), 6.89-6.84 (m, 1H), 6.18 (d, J=8.1 Hz, 1H), 5.00 (dd, J=12.4, 5.5 Hz, 1H), 4.34-4.21 (m, 3H), 4.22-4.13 (m, 1H), 3.75 (d, J=0.8 Hz, 3H), 3.53 (m, 1H), 3.25 (dd, J=8.3, 6.6 Hz, 2H), 2.92-2.81 (m, 1H), 2.66-2.50 (m, 2H), 2.40 (m, 2H), 2.29-2.10 (m, 6H), 2.09-2.00 (m, 4H), 1.89 (s, 3H), 1.87-1.81 (m, 2H), 1.50-1.40 (m, 2H).

### Example 78: 6-Chloro-1-(2-{4-[2-(2,6-dioxopiperidin-3-yl)-1,3-dioxo-2,3-dihydro-1H-isoindol-4-yl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylic acid (309)

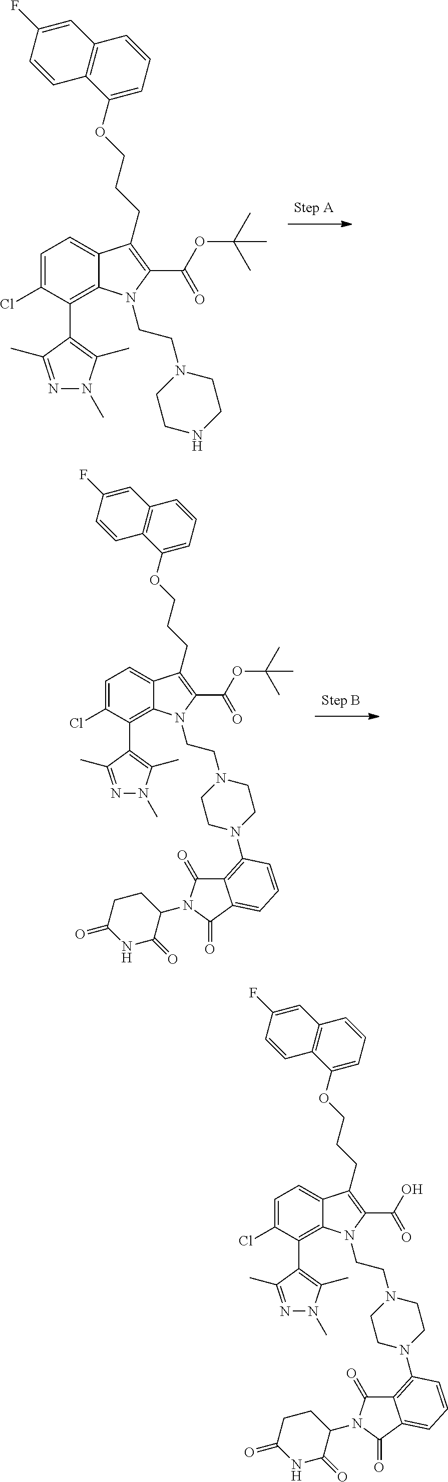 [SMILES: Cc1nn(C)c(C)c1-c1c(Cl)ccc2c(CCCOc3cccc4cc(F)ccc34)c(C(=O)OC(C)(C)C)n(CCN3CCNCC3)c12 ; Cc1nn(C)c(C)c1-c1c(Cl)ccc2c(CCCOc3cccc4cc(F)ccc34)c(C(=O)OC(C)(C)C)n(CCN3CCN(c4cccc5c4C(=O)N(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(c4cccc5c4C(=O)N(C4CCC(=O)NC4=O)C5=O)CC3)c12]

### Step A

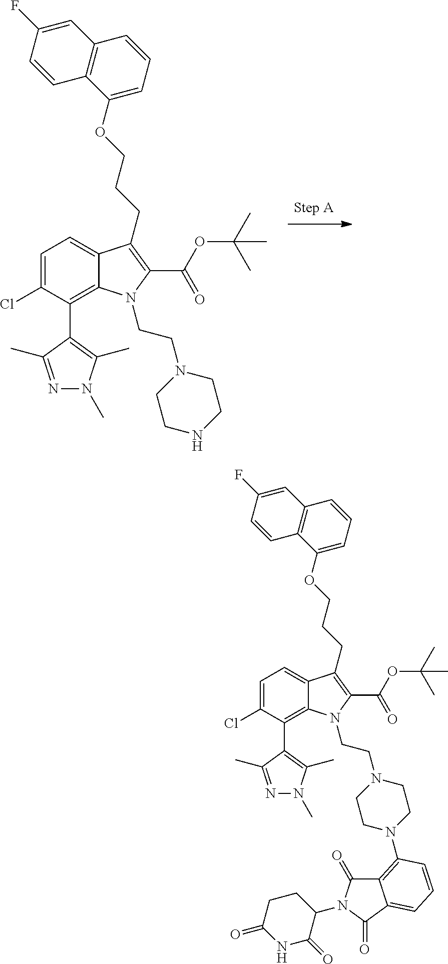 [SMILES: Cc1nn(C)c(C)c1-c1c(Cl)ccc2c(CCCOc3cccc4cc(F)ccc34)c(C(=O)OC(C)(C)C)n(CCN3CCNCC3)c12 ; Cc1nn(C)c(C)c1-c1c(Cl)ccc2c(CCCOc3cccc4cc(F)ccc34)c(C(=O)OC(C)(C)C)n(CCN3CCN(c4cccc5c4C(=O)N(C4CCC(=O)NC4=O)C5=O)CC3)c12]

tert-Butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (25.0 mg, 0.037 mmol) and 2-(2,6-dioxopiperidin-3-yl)-4-fluoro-2,3-dihydro-1H-isoindole-1,3-dione (15.4 mg, 0.056 mmol) were dissolved in DMSO (64 L) and DIPEA (26 μL, 0.148 mmol) was added. Mixture was heated in 90° C. for overnight. Then reaction was concentrated under reduced pressure, and the mixture was diluted with DCM and brine was added. The aqueous layer was extracted with DCM. The combined organic layers were dried with Na₂SO₄ and concentrated under vacuum. The crude (13.5 mg) of tert-butyl 6-chloro-1-(2-{4-[2-(2,6-dioxopiperidin-3-yl)-1,3-dioxo-2,3-dihydro-1H-isoindol-4-yl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate was used for the next step without further purification.

### Step B

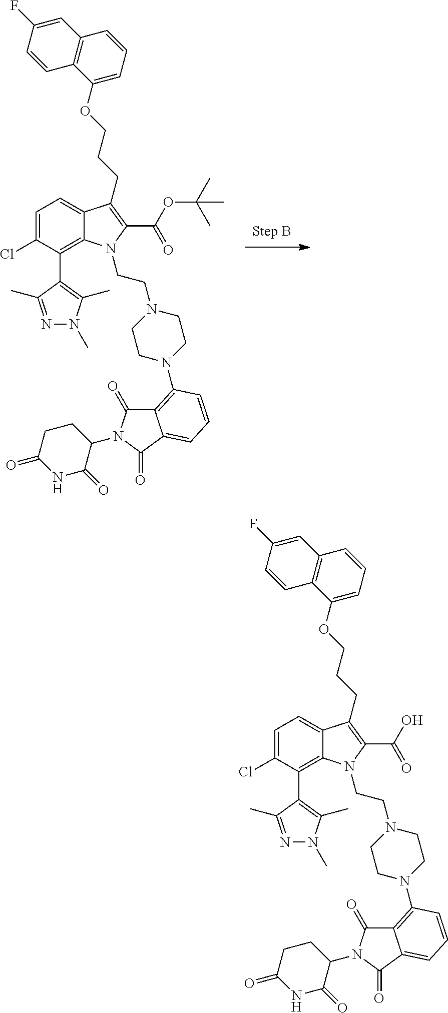 [SMILES: Cc1nn(C)c(C)c1-c1c(Cl)ccc2c(CCCOc3cccc4cc(F)ccc34)c(C(=O)OC(C)(C)C)n(CCN3CCN(c4cccc5c4C(=O)N(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(c4cccc5c4C(=O)N(C4CCC(=O)NC4=O)C5=O)CC3)c12]

To solution of tert-butyl 6-chloro-1-(2-{4-[2-(2,6-dioxopiperidin-3-yl)-1,3-dioxo-2,3-dihydro-1H-isoindol-4-yl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate (13.5 mg, crude) in DCM (0.108 mL) was added TFA (0.108 mL), and mixture was stirred for overnight at room temperature. Then the mixture was diluted with DCM and water was added, and organic solvent was removed under vacuum. Crude was purified by preparative HPLC (C18, H₂O:MeCN+0.1% FA) to give a corresponding 6-chloro-1-(2-{4-[2-(2,6-dioxopiperidin-3-yl)-1,3-dioxo-2,3-dihydro-1H-isoindol-4-yl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylic acid (5.4 mg, 0.006 mmol, 16% over 2 steps) as a yellow solid.

LCMS (ESI+): m/z 874.3 [M+H]⁺

¹H NMR (500 MHz, DMSO) δ=11.07 (d, J=2.5, 1H), 8.22 (dd, J=9.3, 5.9, 1H), 7.74 (d, J=8.5, 1H), 7.68-7.60 (m, 2H), 7.44-7.38 (m, 2H), 7.38-7.31 (m, 2H), 7.28-7.18 (m, 2H), 6.86 (dd, J=6.5, 2.2, 1H), 5.07 (dd, J=12.8, 5.5, 1H), 4.42-4.29 (m, 1H), 4.25-4.10 (m, 3H), 3.77 (s, 3H), 3.20-3.13 (m, 4H), 2.90-2.81 (m, 1H), 2.62-2.52 (m, 2H), 2.46-2.39 (m, 1H), 2.33-2.12 (m, 8H), 2.12-1.96 (m, 5H), 1.88 (s, 3H).

### Example 79. 6-Chloro-1-(2-(4-(2-(2,6-dioxopiperidin-3-yl)-1,3-dioxoisoindolin-4-yl)-2-oxopiperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (310)

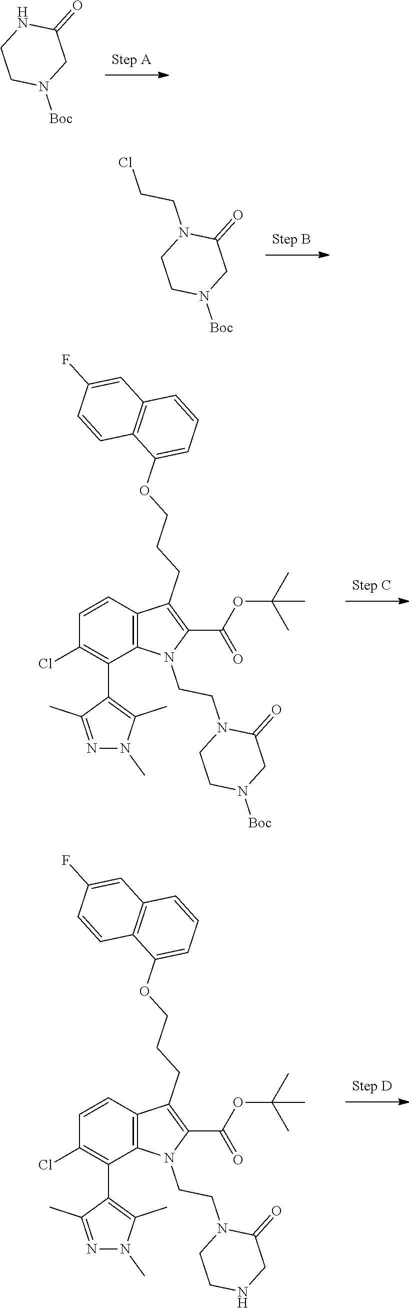

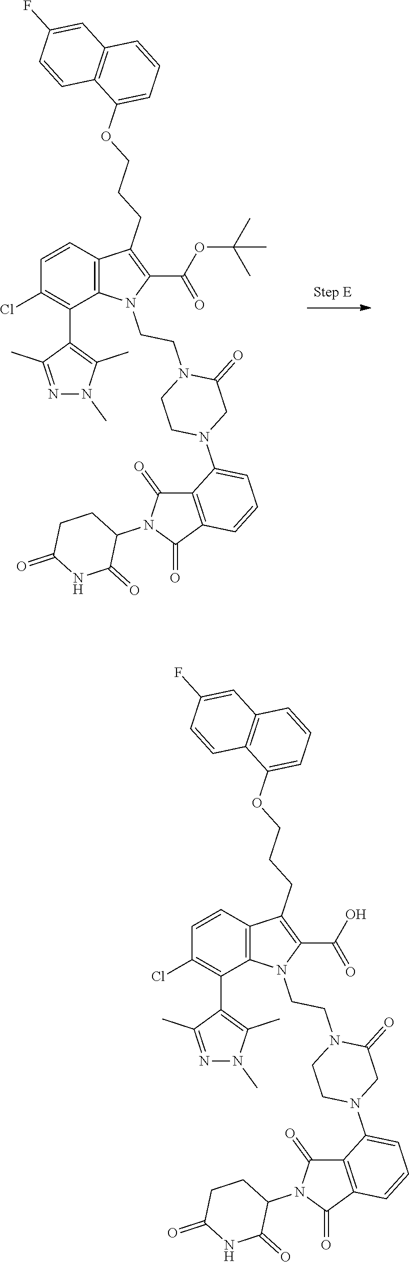

### Step A

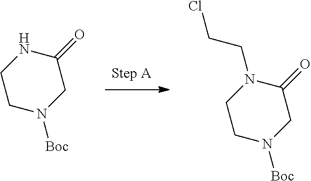 [SMILES: CC(C)(C)OC(=O)N1CCNC(=O)C1 ; CC(C)(C)OC(=O)N1CCN(CCCl)C(=O)C1]

To a stirred solution of tert-butyl 3-oxopiperazine-1-carboxylate (1.0 g, 5.0 mmol) in DMF (20 mL) was added dry (washed by n-hexane) sodium hydride (0.24 g, 10 mmol) portion wise at 0° C. under nitrogen. The reaction mixture was allowed to stir at room temperature for 30 minutes. After that, the reaction mixture was again cooled down to 0° C. and was added 1-bromo-2-chloroethane (0.613 mL, 7.5 mmol) dropwise. The resulting reaction mixture was allowed to stir at room temperature under nitrogen for 3 h. The excess sodium hydride was quenched by addition of ice water and diluted with EtOAc. The organic layer was washed with ice water and brine successively and dried over Na₂SO₄ and then evaporated under reduced pressure to get the crude compound which was then purified by column chromatography (SiO₂, 30-40% EtOAc in hexane) to afford 300 mg (1.14 mmol, 23%) of tert-butyl 4-(2-chloroethyl)-3-oxopiperazine-1-carboxylate as colorless sticky solid.

LCMS (ESI+): m/z 263.2 [M+H]⁺.

### Step B

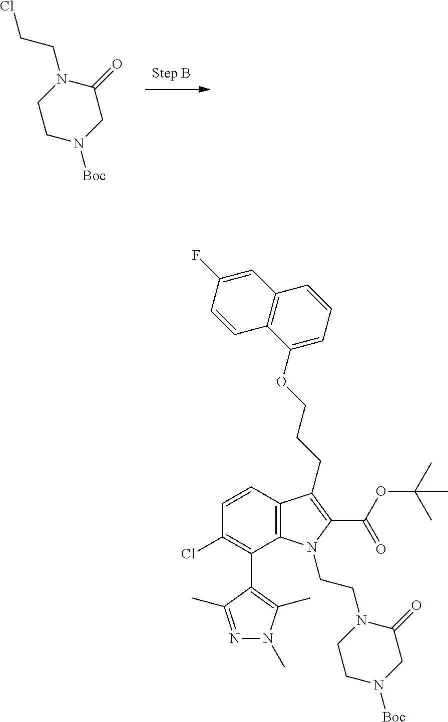 [SMILES: CC(C)(C)OC(=O)N1CCN(CCCl)C(=O)C1 ; Cc1nn(C)c(C)c1-c1c(Cl)ccc2c(CCCOc3cccc4cc(F)ccc34)c(C(=O)OC(C)(C)C)n(CCN3CCN(C(=O)OC(C)(C)C)CC3=O)c12]

To a well stirred solution of tert-butyl 6-chloro-3-(3-((6-fluoronaphthalen-1-yl)oxy)propyl)-7-(1,3,5-trimethyl-1H-pyrazol-4-yl)-1H-indole-2-carboxylate (200 mg, 0.357 mmol) in toluene (1 mL) was added tert-butyl 4-(2-chloroethyl)-3-oxopiperazine-1-carboxylate (467 mg, 1.8 mmol) followed by Cs₂CO₃ (350 mg, 1.07 mmol) and the mixture was allowed to stir at 100° C. for 6 h under nitrogen. After complete consumption of the starting material (monitored by TLC and LCMS) the reaction mixture was diluted with EtOAc, washed successively with water and brine, the organic layer was dried over Na₂SO₄ and evaporated under reduced pressure to get the crude compound, which was then purified by column chromatography over silica gel (50-60% EtOAc in dichloromethane) to get 160 mg (0.2 mmol, 57%) of tert-butyl 1-(2-(4-(tert-butoxycarbonyl)-2-oxopiperazin-1-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate as light brown gummy solid.

LCMS (ESI+): m/z 788.6 [M+H]⁺.

### Step C

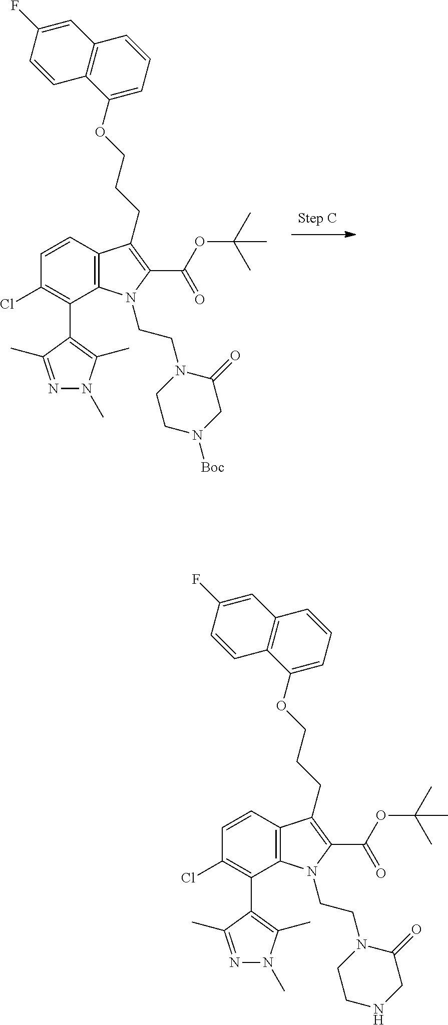 [SMILES: Cc1nn(C)c(C)c1-c1c(Cl)ccc2c(CCCOc3cccc4cc(F)ccc34)c(C(=O)OC(C)(C)C)n(CCN3CCN(C(=O)OC(C)(C)C)CC3=O)c12 ; Cc1nn(C)c(C)c1-c1c(Cl)ccc2c(CCCOc3cccc4cc(F)ccc34)c(C(=O)OC(C)(C)C)n(CCN3CCNCC3=O)c12]

To a well stirred solution of tert-butyl 1-(2-(4-(tert-butoxycarbonyl)-2-oxopiperazin-1-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate (160 mg, 0.2 mmol,) in dioxane (2 mL), was added 6 mL of 4M HCl in dioxane at 0° C. and the mixture stirred for 2 h under nitrogen at the same temperature. Where upon LCMS indicated the reaction was completed, to the reaction mixture was added dropwise cold 1M NaOH solution at 0° C. to maintain the pH˜5 to 6. The aqueous layer was extracted 2-3 times with dichloromethane. The combined organics were dried over Na₂SO₄ and concentrated in vacuo to get the crude compound which was purified by column chromatography (SiO₂, −3-5% MeOH in DCM) to get 55 mg (0.08 mmol, 40%) of tert-butyl 6-chloro-3-(3-((6-fluoronaphthalen-1-yl)oxy)propyl)-1-(2-(2-oxopiperazin-1-yl)ethyl)-7-(1,3,5-trimethyl-1H-pyrazol-4-yl)-1H-indole-2-carboxylate as off white solid.

LCMS (ESI+): m/z 688.5 [M+H]⁺

### Step D

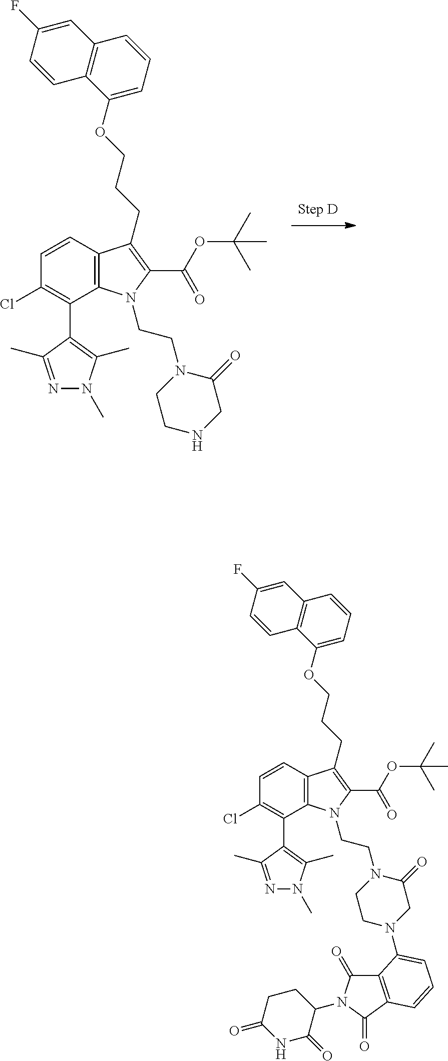 [SMILES: Cc1nn(C)c(C)c1-c1c(Cl)ccc2c(CCCOc3cccc4cc(F)ccc34)c(C(=O)OC(C)(C)C)n(CCN3CCNCC3=O)c12 ; Cc1nn(C)c(C)c1-c1c(Cl)ccc2c(CCCOc3cccc4cc(F)ccc34)c(C(=O)OC(C)(C)C)n(CCN3CCN(c4cccc5c4C(=O)N(C4CCC(=O)NC4=O)C5=O)CC3=O)c12]

tert-Butyl 6-chloro-3-{3-[(6-fluoronaphthalen-1-yl)oxy]propyl}-1-[2-(2-oxopiperazin-1-yl)ethyl]-7-(1,3,5-trimethyl-1H-pyrazol-4-yl)-1H-indole-2-carboxylate (25.0 mg, 0.036 mmol) and 2-(2,6-dioxopiperidin-3-yl)-4-fluoro-2,3-dihydro-1H-isoindole-1,3-dione (15.1 mg, 0.054 mmol) were dissolved in dry DMSO (0.726 mL) and DIPEA (0.025 mL, 0.145 mmol) was added. Reaction, (monitored with LCMS) was stirred at 90° C. for 48 h. After complete conversion of the starting material the solution was diluted with DMSO and purified with reverse phase flash chromatography (C18, H₂O:MeCN+0.1% FA) to give tert-butyl 6-chloro-1-(2-{4-[2-(2,6-dioxopiperidin-3-yl)-1,3-dioxo-2,3-dihydro-1H-isoindol-4-yl]-2-oxopiperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate (13.2 mg, 0.014 mmol, 38.5%) as yellow solid.

LCMS (ESI+): m/z 943.6 [M+H]⁺.

### Step E

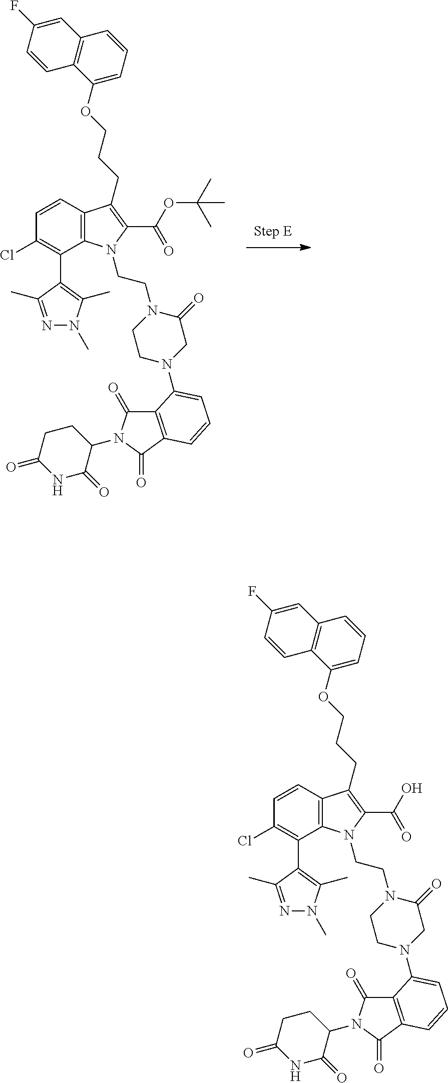 [SMILES: Cc1nn(C)c(C)c1-c1c(Cl)ccc2c(CCCOc3cccc4cc(F)ccc34)c(C(=O)OC(C)(C)C)n(CCN3CCN(c4cccc5c4C(=O)N(C4CCC(=O)NC4=O)C5=O)CC3=O)c12 ; Cc1nn(C)c(C)c1-c1c(Cl)ccc2c(CCCOc3cccc4cc(F)ccc34)c(C(=O)O)n(CCN3CCN(c4cccc5c4C(=O)N(C4CCC(=O)NC4=O)C5=O)CC3=O)c12]

tert-Butyl 6-chloro-1-(2-{4-[2-(2,6-dioxopiperidin-3-yl)-1,3-dioxo-2,3-dihydro-1H-isoindol-4-yl]-2-oxopiperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate (13.2 mg, 0.014 mmol) was dissolved in dry DCM (0.50 mL) under argon atmosphere and TFA (0.500 mL, 6.529 mmol) was added. Reaction (monitored with LCMS) was stirred for 16 h in room temperature. After complete consumption of the starting material the solution was concentrated under reduced pressure and the resulting residue dissolved in DMSO. Crude product was purified with preparative HPLC (H₂O:MeCN+0.1% FA) to give 6-chloro-1-(2-(4-(2-(2,6-dioxopiperidin-3-yl)-1,3-dioxoisoindolin-4-yl)-2-oxopiperazin-1-yl)ethyl)-3-(3-((6-fluoronaphthalen-1-yl)oxy)propyl)-7-(1,3,5-trimethyl-1H-pyrazol-4-yl)-1H-indole-2-carboxylicacid (6.9 mg, 0.008 mmol, 55.3%) as yellow solid.

LCMS (ESI+): m/z 888.3 [M+H]⁺.

¹H NMR (500 MHz, DMSO) δ 13.31 (s, 1H), 11.07 (s, 1H), 8.24 (dd, J=9.3, 5.8 Hz, 1H), 7.75 (d, J=8.5 Hz, 1H), 7.68-7.61 (m, 2H), 7.47-7.38 (m, 2H), 7.38-7.30 (m, 2H), 7.25 (d, J=8.5 Hz, 1H), 7.22 (d, J=8.5 Hz, 1H), 6.87 (dd, J=5.4, 3.3 Hz, 1H), 5.06 (dd, J=12.8, 5.5 Hz, 1H), 4.50-4.31 (m, 1H), 4.31-4.12 (m, 3H), 3.79 (s, 2H), 3.77 (s, 3H), 3.49-3.42 (m, 2H), 3.26-3.20 (m, 2H), 3.14-3.04 (m, 1H), 2.98-2.79 (m, 4H), 2.61-2.54 (m, 1H), 2.48-2.43 (m, 1H), 2.26-2.14 (m, 2H), 2.06 (d, J=3.9 Hz, 3H), 2.03-1.95 (m, 1H), 1.88 (d, J=3.7 Hz, 3H).

### Example 80. 6-Chloro-1-{2-[4-(2-{[2-(2,6-dioxopiperidin-3-yl)-1-oxo-2,3-dihydro-1H-isoindol-4-yl]oxy}-2-methylpropanoyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (311)

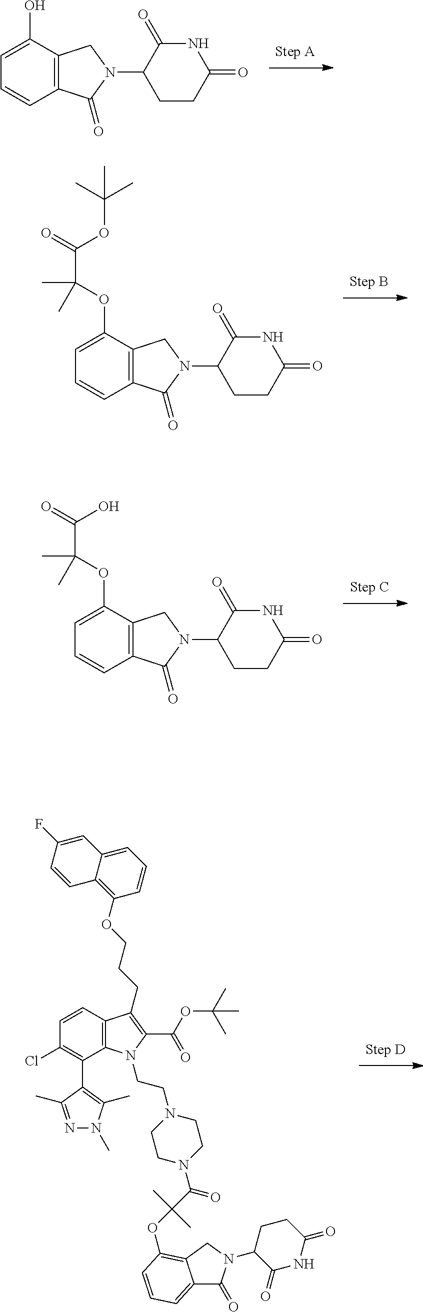

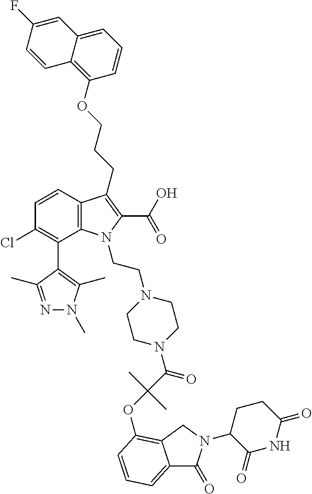 [SMILES: Cc1nn(C)c(C)c1-c1c(Cl)ccc2c(CCCOc3cccc4cc(F)ccc34)c(C(=O)O)n(CCN3CCN(C(=O)C(C)(C)Oc4cccc5c4CN(C4CCC(=O)NC4=O)C5=O)CC3)c12]

### Step A

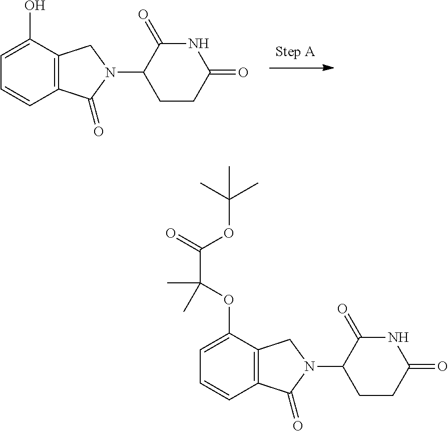 [SMILES: O=C1CCC(N2Cc3c(O)cccc3C2=O)C(=O)N1 ; CC(C)(C)OC(=O)C(C)(C)Oc1cccc2c1CN(C1CCC(=O)NC1=O)C2=O]

To the mixture of 3-(4-hydroxy-1-oxoisoindolin-2-yl)piperidine-2,6-dione (150 mg, 0.576 mmol), K₂CO₃ (319 mg, 0.002 mmol) and MgSO₄ (71 mg, 0.576 mmol), DMF (1 mL) followed by tert-butyl-2-bromoisobutyrate (325 μL, 1.7 mmol) were added under nitrogen. The resulting mixture was heated at 80° C. for 3 hours. The reaction was cool down to room temperature then quenched with water and brine and extracted with EtOAc. The crude was purified by flash column chromatography (SiO₂, 0-100% EtOAc in cyclohexane) to obtain tert-butyl 2-((2-(2,6-dioxopiperidin-3-yl)-1-oxoisoindolin-4-yl)oxy)-2-methylpropanoate (108 mg, 0.268 mmol, 47%) as a colorless oil.

LCMS (ESI+): m/z 403.2 [M+H]⁺

### Step B

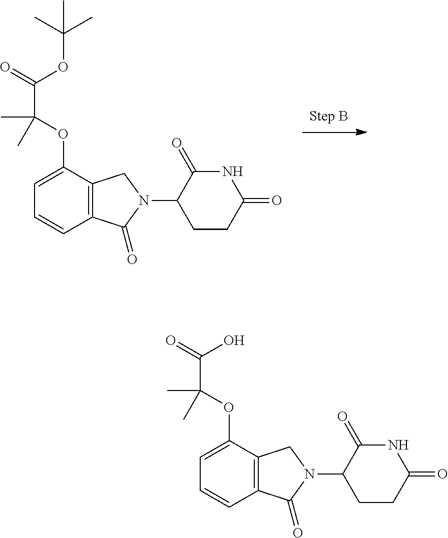 [SMILES: CC(C)(C)OC(=O)C(C)(C)Oc1cccc2c1CN(C1CCC(=O)NC1=O)C2=O ; CC(C)(Oc1cccc2c1CN(C1CCC(=O)NC1=O)C2=O)C(=O)O]

To a solution of tert-butyl 2-((2-(2,6-dioxopiperidin-3-yl)-1-oxoisoindolin-4-yl)oxy)-2-methylpropanoate (150 mg, 0.373 mmol) in DCM (7.5 mL) at 0° C. was added TFA (1.15 mL). The mixture was allowed to warm up to room temperature and stirred for 5 hours. The volatiles were removed in vacuo. The crude residue was dissolved in MeOH and concentrated in vacuo. The operation was repeated three times to remove remaining TFA. The product, 2-((2-(2,6-dioxopiperidin-3-yl)-1-oxoisoindolin-4-yl)oxy)-2-methylpropanoic acid, was isolated (121 mg, 0.349 mmol, 94%) as a white solid.

LCMS (ESI+): m/z 347.0 [M+H]⁺

### Step C

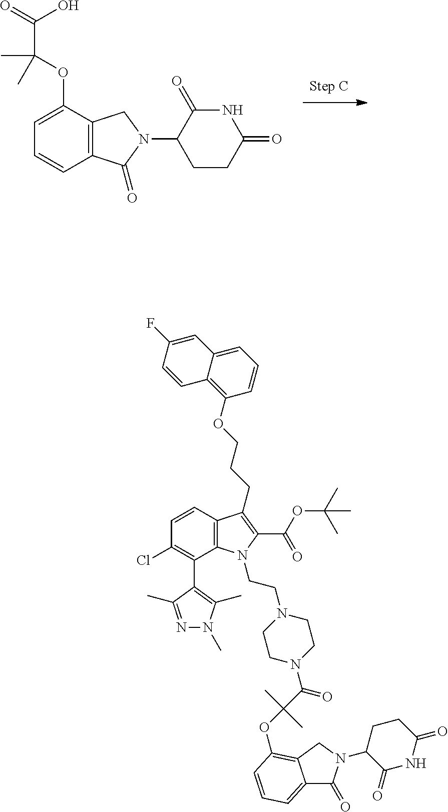 [SMILES: CC(C)(Oc1cccc2c1CN(C1CCC(=O)NC1=O)C2=O)C(=O)O ; Cc1nn(C)c(C)c1-c1c(Cl)ccc2c(CCCOc3cccc4cc(F)ccc34)c(C(=O)OC(C)(C)C)n(CCN3CCN(C(=O)C(C)(C)Oc4cccc5c4CN(C4CCC(=O)NC4=O)C5=O)CC3)c12]

To a solution of tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (20.0 mg, 0.030 mmol) and 2-((2-(2,6-dioxopiperidin-3-yl)-1-oxoisoindolin-4-yl)oxy)-2-methylpropanoic acid (15.4 mg, 0.044 mmol) in dry DMF (2.0 mL) were added HATU (22.6 mg, 0.059 mmol) and DIPEA (0.016 mL, 0.089 mmol). The reaction was stirred at room temperature for 15 min. The crude was diluted in DCM and washed with brine. The organic layer was dried over MgSO₄, filtered and concentrated in vacuo to obtain tert-butyl 6-chloro-1-{2-[4-(2-{[2-(2,6-dioxopiperidin-3-yl)-1-oxo-2,3-dihydro-1H-isoindol-4-yl]oxy}-2-methylpropanoyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (49 mg, crude) as an yellow oil, which was used to next step without further purification.

LCMS (ESI+): m/z 1003.2 [M+H]⁺

### Step D

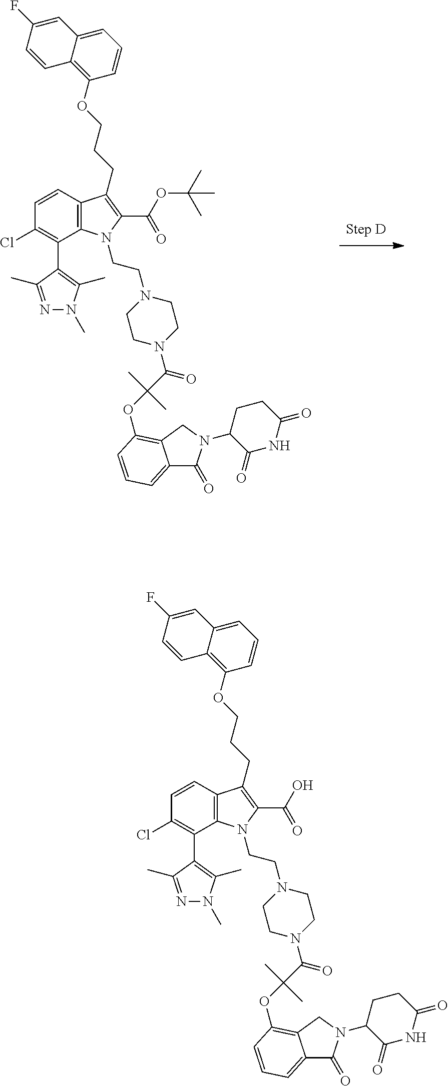 [SMILES: Cc1nn(C)c(C)c1-c1c(Cl)ccc2c(CCCOc3cccc4cc(F)ccc34)c(C(=O)OC(C)(C)C)n(CCN3CCN(C(=O)C(C)(C)Oc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)C(C)(C)Oc4cccc5c4CN(C4CCC(=O)NC4=O)C5=O)CC3)c12]

To a solution of tert-butyl 6-chloro-1-{2-[4-(2-{[2-(2,6-dioxopiperidin-3-yl)-1-oxo-2,3-dihydro-1H-isoindol-4-yl]oxy}-2-methylpropanoyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (49 mg, crude) in DCM (1 mL) was added TFA (1.0 mL) and the mixture was stirred for next 18 h at RT. The crude was concentrated in vacuo and purified by preparative HPLC (C18, H₂O:MeCN+0.1% FA) to obtain 6-chloro-1-{2-[4-(2-{[2-(2,6-dioxopiperidin-3-yl)-1-oxo-2,3-dihydro-1H-isoindol-4-yl]oxy}-2-methylpropanoyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (16.0 mg, 0.017 mmol, 56.7% over two steps) as a white solid.

LCMS (ESI+): m/z 946.15 [M+H]⁺

¹H NMR (500 MHz, DMSO, 353 K) δ 10.65 (s, 1H), 8.22 (dd, J=9.2, 5.9 Hz, 1H), 7.67 (d, J=8.5 Hz, 1H), 7.58 (dd, J=10.4, 2.6 Hz, 1H), 7.43-7.38 (m, 3H), 7.35-7.29 (m, 2H), 7.20-7.16 (m, 1H), 6.90-6.87 (m, 1H), 6.85 (dd, J=6.4, 2.2 Hz, 1H), 5.06 (ddd, J=12.9, 7.6, 5.2 Hz, 1H), 4.37 (dd, J=17.2, 7.7 Hz, 1H), 4.28 (dd, J=17.2, 3.8 Hz, 1H), 4.23-4.17 (m, 3H), 4.12-4.06 (m, 1H), 3.68 (s, 3H), 3.52 (bs, 4H), 3.24 (dd, J=8.5, 6.5 Hz, 2H), 2.93-2.84 (m, 1H), 2.66-2.60 (m, 1H), 2.47-2.38 (m, 1H), 2.24-2.17 (m, 2H), 2.08-2.02 (m, 1H), 1.99-1.88 (m, 9H), 1.82 (d, J=6.6 Hz, 3H), 1.58 (d, J=2.5 Hz, 3H), 1.57 (s, 3H).

### Example 81. 6-Chloro-1-(2-{4-[(2S)-2-({2-[(3S)-2,6-dioxopiperidin-3-yl]-1-oxo-2,3-dihydro-1H-isoindol-4-yl}oxy)propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylic acid (313)

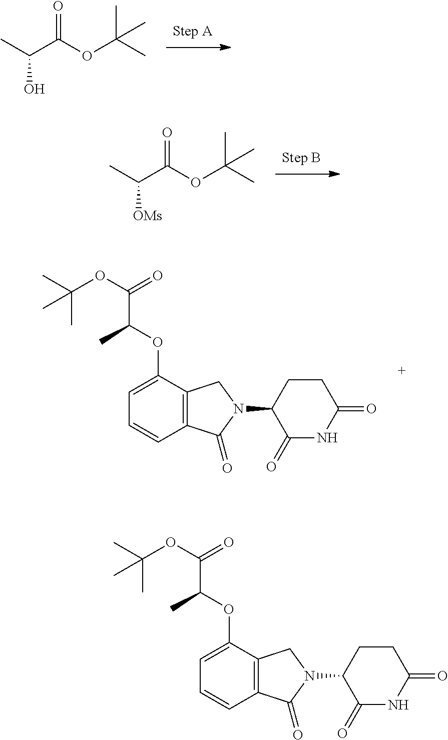

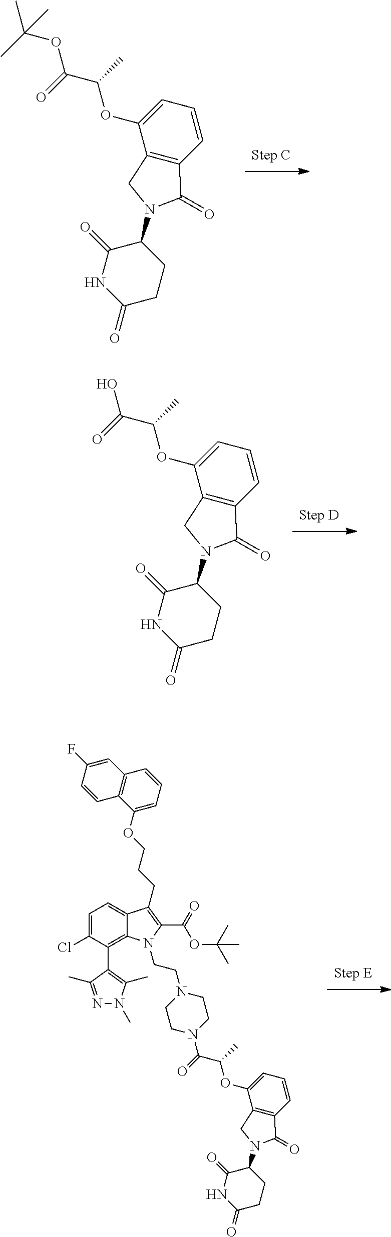 [SMILES: C[C@H](Oc1cccc2c1CN([C@H]1CCC(=O)NC1=O)C2=O)C(=O)OC(C)(C)C ; C[C@H](Oc1cccc2c1CN([C@H]1CCC(=O)NC1=O)C2=O)C(=O)O ; Cc1nn(C)c(C)c1-c1c(Cl)ccc2c(CCCOc3cccc4cc(F)ccc34)c(C(=O)OC(C)(C)C)n(CCN3CCN(C(=O)[C@H](C)Oc4cccc5c4CN([C@H]4CCC(=O)NC4=O)C5=O)CC3)c12]

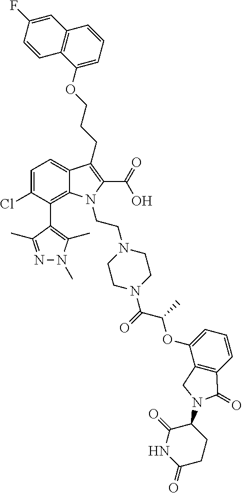 [SMILES: Cc1nn(C)c(C)c1-c1c(Cl)ccc2c(CCCOc3cccc4cc(F)ccc34)c(C(=O)O)n(CCN3CCN(C(=O)[C@H](C)Oc4cccc5c4CN([C@H]4CCC(=O)NC4=O)C5=O)CC3)c12]

### Step A

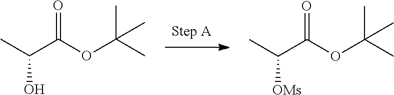

To a stirred solution of tert-butyl (R)-2-hydroxypropanoate (500 mg, 3.42 mmol) in DCM (15 mL) was added MsCl (0.530 mL, 6.5 mmol) followed by Et₃N (1.04 mL, 10.27 mmol) at 0° C. under nitrogen and the reaction mixture was allowed to stir at same temperature for 1 h. After complete consumption of the starting material (monitored by TLC) the reaction mixture was diluted with EtOAc, washed successively with water and brine, the organic layer was dried over Na₂SO₄ and evaporated under reduced pressure to get the crude compound, which was then purified by column chromatography (SiO₂, 10-20% EtOAc in hexane) to obtain tert-butyl (R)-2-((methylsulfonyl)oxy)propanoate (500 mg, 2.23 mmol, 65%) as white solid.

### Step B

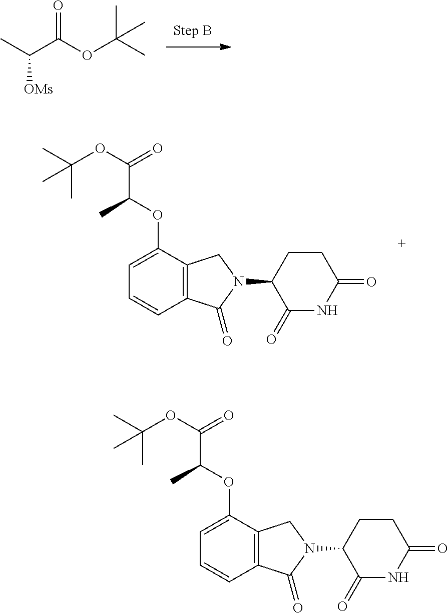 [SMILES: C[C@@H](OS(C)(=O)=O)C(=O)OC(C)(C)C ; C[C@H](Oc1cccc2c1CN([C@H]1CCC(=O)NC1=O)C2=O)C(=O)OC(C)(C)C ; C[C@H](Oc1cccc2c1CN([C@@H]1CCC(=O)NC1=O)C2=O)C(=O)OC(C)(C)C]

To a well stirred solution of tert-butyl (R)-2-((methylsulfonyl)oxy)propanoate (500 mg, 2.23 mmol) in DMF (8 mL) was added KHCO₃ (184.6 mg, 1.846 mmol) and 2-(2,6-dioxopiperidin-3-yl)-4-hydroxyisoindoline-1,3-dione (400 mg, 1.54 mmol) successively at room temperature under nitrogen. The reaction mixture was allowed to stir at 60° C. for 3 h. After complete consumption of the starting material (monitored by LCMS) the reaction mixture was diluted with EtOAc, washed successively with water and brine. The organic layer was dried over Na₂SO₄ and evaporated under reduced pressure to get the crude compound, which was then purified by column chromatography (SiO₂, 50-60% EtOAc in DCM) to get 300 mg tert-butyl (2S)-2-((2-(2,6-dioxopiperidin-3-yl)-1-oxoisoindolin-4-yl)oxy)propanoate. This mixture of enantiomers was purified by preparative chiral HPLC for separation tert-butyl (S)-2-((2-((S)-2,6-dioxopiperidin-3-yl)-1-oxoisoindolin-4-yl)oxy)propanoate (150 mg, 0.39 mmol, 25%)

LCMS (ESI+): m/z 389.1 [M+H]⁺

and tert-butyl (S)-2-((2-(R)-2,6-dioxopiperidin-3-yl)-1-oxoisoindolin-4-yl)oxy)propanoate (100 mg, 0.26 mmol, 17%).

LCMS (ESI+): m/z 389.2 [M+H]⁺

Absolute stereochemistry of compounds is arbitrarily assigned.

### Step C

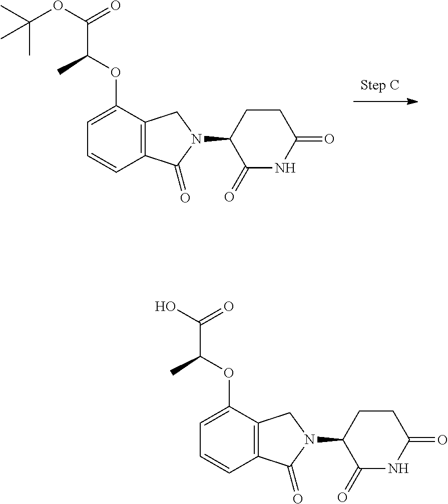 [SMILES: C[C@H](Oc1cccc2c1CN([C@H]1CCC(=O)NC1=O)C2=O)C(=O)OC(C)(C)C ; C[C@H](Oc1cccc2c1CN([C@H]1CCC(=O)NC1=O)C2=O)C(=O)O]

To a stirred solution of tert-butyl (S)-2-((2-((S)-2,6-dioxopiperidin-3-yl)-1-oxoisoindolin-4-yl)oxy)propanoate (150 mg, 0.39 mmol) in DCM (4 mL) was added TFA (2 mL) drop wise at 0° C. under nitrogen and the reaction mixture was allowed to stir at room temperature for 16 h. After consuming of starting material (monitored by LCMS) the volatiles were evaporated under reduced pressure to get the crude compound, which was then purified by triturating it by Et₂O and pentane to afford (5)-2-((2-((S)-2,6-dioxopiperidin-3-yl)-1-oxoisoindolin-4-yl)oxy)propanoic acid (70 mg, 0.21 mmol, 54%) as brown solid.

LCMS (ESI+): m/z 333.1 [M+H]⁺

### Step D

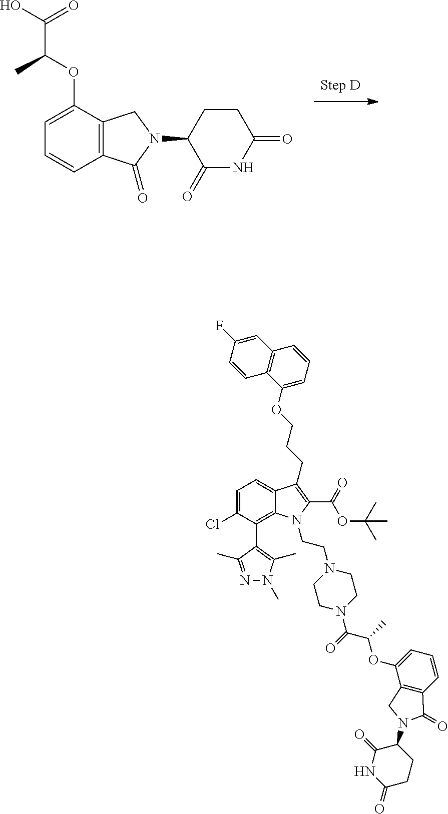 [SMILES: C[C@H](Oc1cccc2c1CN([C@H]1CCC(=O)NC1=O)C2=O)C(=O)O ; Cc1nn(C)c(C)c1-c1c(Cl)ccc2c(CCCOc3cccc4cc(F)ccc34)c(C(=O)OC(C)(C)C)n(CCN3CCN(C(=O)[C@H](C)Oc4cccc5c4CN([C@H]4CCC(=O)NC4=O)C5=O)CC3)c12]

To a solution of tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (15.0 mg, 0.022 mmol) and (5)-2-((2-((S)-2,6-dioxopiperidin-3-yl)-1-oxoisoindolin-4-yl)oxy)propanoic acid (8.9 mg, 0.027 mmol) in dry DMF (2.0 mL) were added HATU (16.9 mg, 0.044 mmol) and DIPEA (0.019 mL, 0.111 mmol). The reaction was stirred at room temperature for 15 min. The crude was diluted in DCM and washed with brine. The organic layer was dried over MgSO₄, filtered and concentrated in vacuo to obtain tert-butyl 6-chloro-1-(2-{4-[(2S)-2-({2-[(3S)-2,6-dioxopiperidin-3-yl]-1-oxo-2,3-dihydro-1H-isoindol-4-yl}oxy)propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate (29 mg, crude) as an yellow oil, which was used to next step without further purification.

LCMS (ESI+): m/z 988.7 [M+H]⁺

### Step E

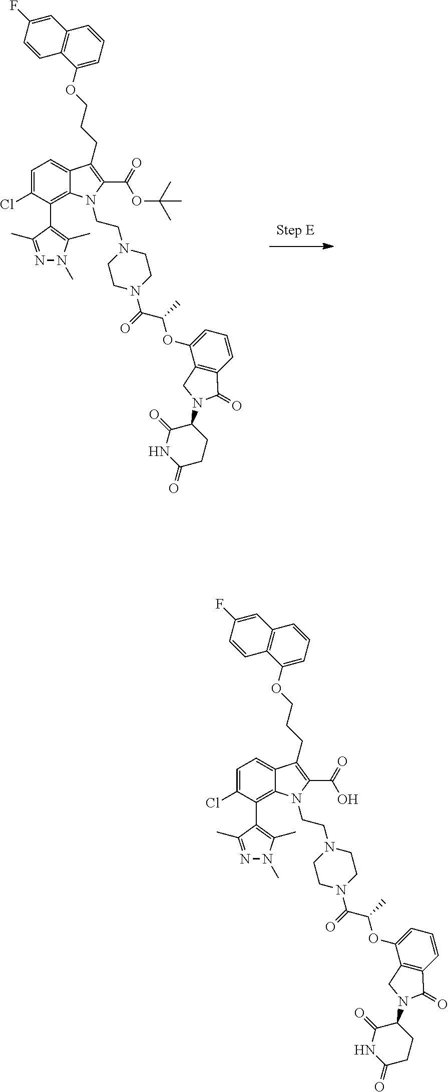 [SMILES: Cc1nn(C)c(C)c1-c1c(Cl)ccc2c(CCCOc3cccc4cc(F)ccc34)c(C(=O)OC(C)(C)C)n(CCN3CCN(C(=O)[C@H](C)Oc4cccc5c4CN([C@H]4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)[C@H](C)Oc4cccc5c4CN([C@H]4CCC(=O)NC4=O)C5=O)CC3)c12]

To a solution of tert-butyl 6-chloro-1-(2-{4-[(2S)-2-({2-[(3S)-2,6-dioxopiperidin-3-yl]-1-oxo-2,3-dihydro-1H-isoindol-4-yl}oxy)propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate (29 mg, crude) in DCM (1.0 mL) was added TFA (1.0 mL, 13.059 mmol). The reaction mixture was stirred for 18 h at RT. The crude was concentrated in vacuo and purified by preparative HPLC (C18, H₂O:MeCN+0.1% FA) to obtain 6-chloro-1-(2-{4-[(2S)-2-({2-[(3S)-2,6-dioxopiperidin-3-yl]-1-oxo-2,3-dihydro-1H-isoindol-4-yl}oxy)propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylic acid (16.5 mg, 0.018 mmol, 82% over two steps) as a white solid.

LCMS (ESI+): m/z 932.15 [M+H]⁺

¹H NMR (500 MHz, DMSO, 353 K) δ 10.64 (s, 1H), 8.23 (dd, J=9.3, 5.8 Hz, 1H), 7.71 (d, J=8.5 Hz, 1H), 7.58 (dd, J=10.4, 2.6 Hz, 1H), 7.47-7.37 (m, 3H), 7.36-7.28 (m, 2H), 7.22 (d, J=8.5 Hz, 1H), 7.05 (d, J=8.1 Hz, 1H), 6.86 (dd, J=6.1, 2.6 Hz, 1H), 5.31 (qd, J=6.5, 3.0 Hz, 1H), 5.05 (dd, J=13.1, 5.2 Hz, 1H), 4.38 (d, J=17.2 Hz, 1H), 4.32-4.21 (m, 4H), 4.22-4.12 (m, 1H), 3.73 (s, 3H), 3.44-3.37 (m, 4H), 3.28 (dd, J=8.5, 6.5 Hz, 2H), 2.88 (ddd, J=17.3, 13.5, 5.6 Hz, 1H), 2.66-2.58 (m, 1H), 2.48-2.38 (m, 1H), 2.28-2.19 (m, 2H), 2.13-2.00 (m, 7H), 1.99 (d, J=2.9 Hz, 3H), 1.87 (d, J=1.9 Hz, 3H), 1.45 (d, J=6.5 Hz, 3H).

### Example 82. 6-Chloro-1-(2-{4-[(2S)-2-({2-[(3R)-2,6-dioxopiperidin-3-yl]-1-oxo-2,3-dihydro-1H-isoindol-4-yl}oxy)propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylic acid (314)

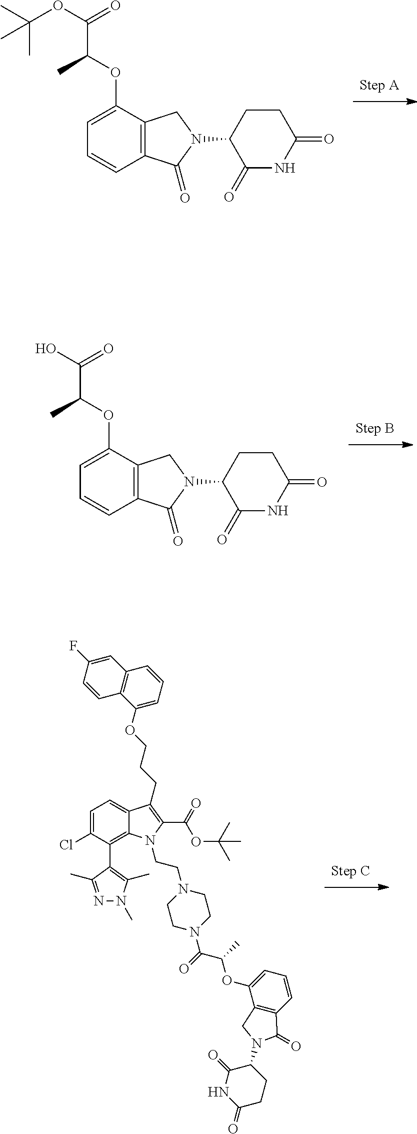 [SMILES: C[C@H](Oc1cccc2c1CN([C@@H]1CCC(=O)NC1=O)C2=O)C(=O)OC(C)(C)C ; C[C@H](Oc1cccc2c1CN([C@@H]1CCC(=O)NC1=O)C2=O)C(=O)O ; Cc1nn(C)c(C)c1-c1c(Cl)ccc2c(CCCOc3cccc4cc(F)ccc34)c(C(=O)OC(C)(C)C)n(CCN3CCN(C(=O)[C@H](C)Oc4cccc5c4CN([C@@H]4CCC(=O)NC4=O)C5=O)CC3)c12]

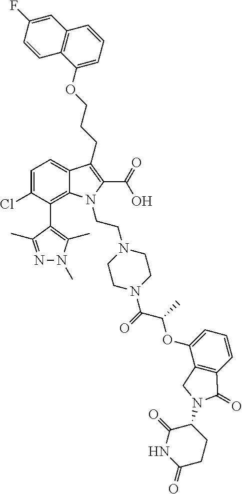 [SMILES: Cc1nn(C)c(C)c1-c1c(Cl)ccc2c(CCCOc3cccc4cc(F)ccc34)c(C(=O)O)n(CCN3CCN(C(=O)[C@H](C)Oc4cccc5c4CN([C@@H]4CCC(=O)NC4=O)C5=O)CC3)c12]

### Step A

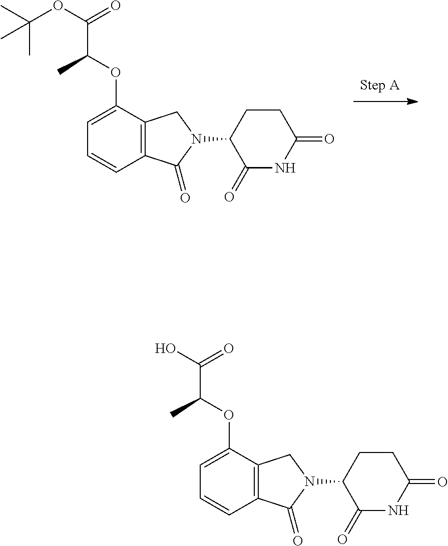 [SMILES: C[C@H](Oc1cccc2c1CN([C@@H]1CCC(=O)NC1=O)C2=O)C(=O)OC(C)(C)C ; C[C@H](Oc1cccc2c1CN([C@@H]1CCC(=O)NC1=O)C2=O)C(=O)O]

To a stirred solution of tert-butyl (S)-2-((2-(R)-2,6-dioxopiperidin-3-yl)-1-oxoisoindolin-4-yl)oxy)propanoate (150 mg, 0.39 mmol) in DCM (4 mL) was added TFA (2 mL) drop wise at 0° C. under nitrogen and the reaction mixture was allowed to stir at room temperature for 16 h. After consuming of starting material (monitored by LCMS) the volatiles were evaporated under reduced pressure to get the crude compound, which was then purified by triturating it by Et₂O and pentane to afford (5)-2-((2-((R)-2,6-dioxopiperidin-3-yl)-1-oxoisoindolin-4-yl)oxy)propanoic acid (63.9 mg, 0.19 mmol, 75%) as brown solid.

LCMS (ESI+): m/z 333.1 [M+H]⁺

### Step B

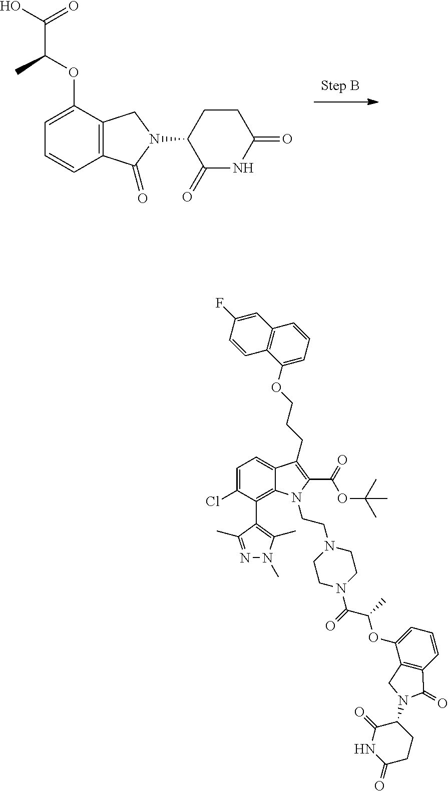 [SMILES: C[C@H](Oc1cccc2c1CN([C@@H]1CCC(=O)NC1=O)C2=O)C(=O)O ; Cc1nn(C)c(C)c1-c1c(Cl)ccc2c(CCCOc3cccc4cc(F)ccc34)c(C(=O)OC(C)(C)C)n(CCN3CCN(C(=O)[C@H](C)Oc4cccc5c4CN([C@@H]4CCC(=O)NC4=O)C5=O)CC3)c12]

To a solution of tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (15.0 mg, 0.022 mmol) and (5)-2-((2-((R)-2,6-dioxopiperidin-3-yl)-1-oxoisoindolin-4-yl)oxy)propanoic acid (8.9 mg, 0.027 mmol) in dry DMF (2.0 mL) were added HATU (16.9 mg, 0.044 mmol) and DIPEA (0.019 mL, 0.111 mmol). The reaction was stirred at room temperature for 15 min. The crude was diluted in DCM and washed with brine. The organic layer was dried over MgSO₄, filtered and concentrated in vacuo to obtain tert-butyl 6-chloro-1-(2-{4-[(2S)-2-({2-[(3R)-2,6-dioxopiperidin-3-yl]-1-oxo-2,3-dihydro-1H-isoindol-4-yl}oxy)propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate (47 mg, crude) as an yellow oil, which was used to next step without further purification.

LCMS (ESI+): m/z 988.6 [M+H]⁺

### Step C

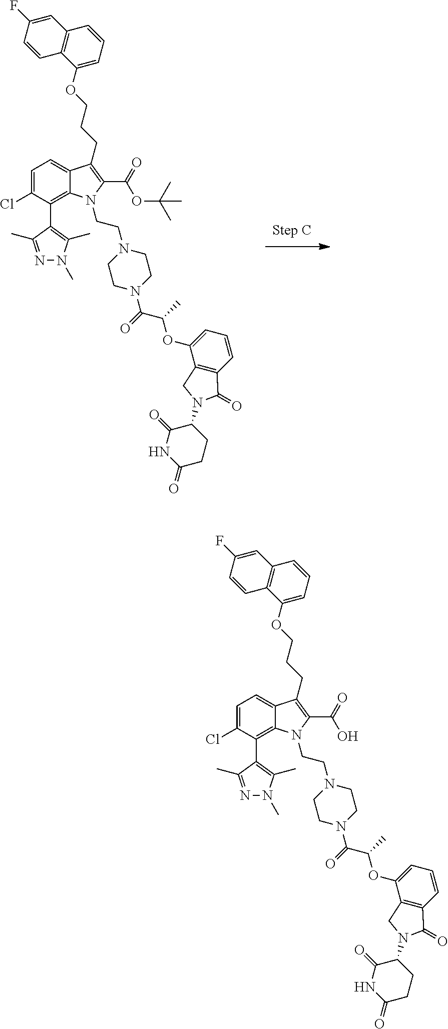 [SMILES: Cc1nn(C)c(C)c1-c1c(Cl)ccc2c(CCCOc3cccc4cc(F)ccc34)c(C(=O)OC(C)(C)C)n(CCN3CCN(C(=O)[C@H](C)Oc4cccc5c4CN([C@@H]4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)[C@H](C)Oc4cccc5c4CN([C@@H]4CCC(=O)NC4=O)C5=O)CC3)c12]

To a solution of tert-butyl 6-chloro-1-(2-{4-[(2S)-2-({2-[(3R)-2,6-dioxopiperidin-3-yl]-1-oxo-2,3-dihydro-1H-isoindol-4-yl}oxy)propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate (47 mg, crude) in DCM (1.0 mL) was added TFA (1.0 mL). The reaction mixture was stirred for 18 h at RT. The crude was concentrated in vacuo and purified by preparative HPLC (C18, H₂O:MeCN+0.1% FA) to obtain 6-chloro-1-(2-{4-[(2S)-2-({2-[(3R)-2,6-dioxopiperidin-3-yl]-1-oxo-2,3-dihydro-1H-isoindol-4-yl}oxy)propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylic acid (18.5 mg, 0.020 mmol, 91% over two steps) as a white solid.

LCMS (ESI+): m/z 932.15 [M+H]⁺

¹H NMR (500 MHz, DMSO, 353 K) δ 10.64 (s, 1H), 8.23 (dd, J=9.2, 5.9 Hz, 1H), 7.71 (d, J=8.6 Hz, 1H), 7.58 (dd, J=10.4, 2.6 Hz, 1H), 7.46-7.38 (m, 3H), 7.36-7.28 (m, 2H), 7.22 (d, J=8.5 Hz, 1H), 7.05 (dd, J=8.0, 3.8 Hz, 1H), 6.87 (dd, J=6.2, 2.5 Hz, 1H), 5.32 (q, J=6.5 Hz, 1H), 5.04 (dd, J=13.0, 5.2 Hz, 1H), 4.38 (d, J=17.2 Hz, 1H), 4.35-4.21 (m, 4H), 4.21-4.12 (m, 1H), 3.73 (d, J=9.2 Hz, 3H), 3.46-3.35 (m, 4H), 3.31-3.25 (m, 2H), 2.87 (ddd, J=17.3, 13.4, 5.5 Hz, 1H), 2.67-2.59 (m, 1H), 2.49-2.39 (m, 1H), 2.28-2.19 (m, 2H), 2.12-1.99 (m, 7H), 1.99 (d, J=0.8 Hz, 3H), 1.88 (d, J=1.0 Hz, 3H), 1.48-1.37 (m, 3H).

### Example 83. 6-Chloro-1-(2-(4-((2S)-2-((2-(2,6-dioxopiperidin-3-yl)-1-oxoisoindolin-4-yl)oxy)propanoyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (312)

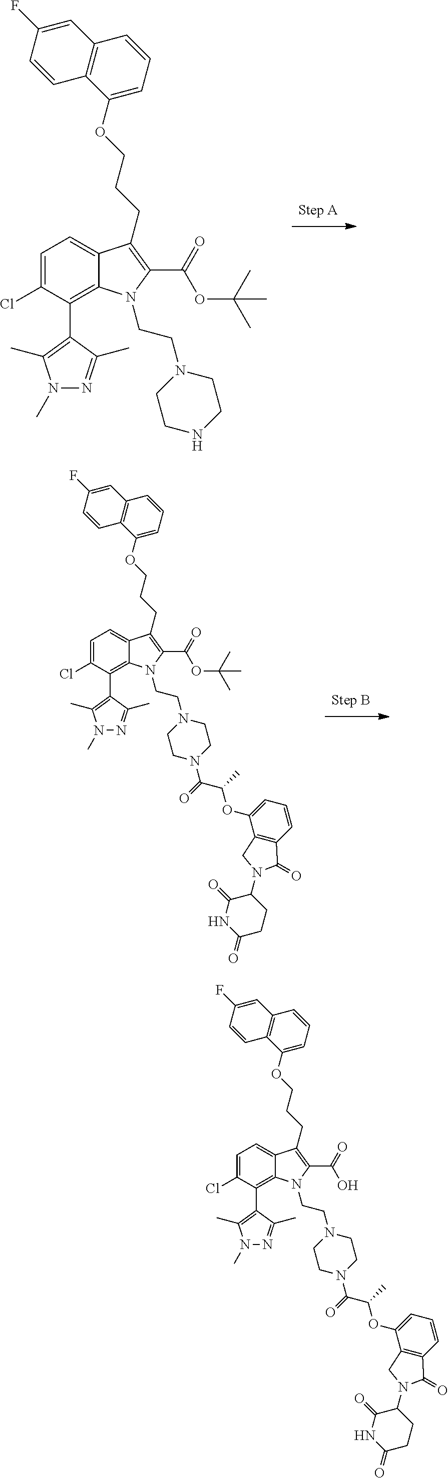

### Step A

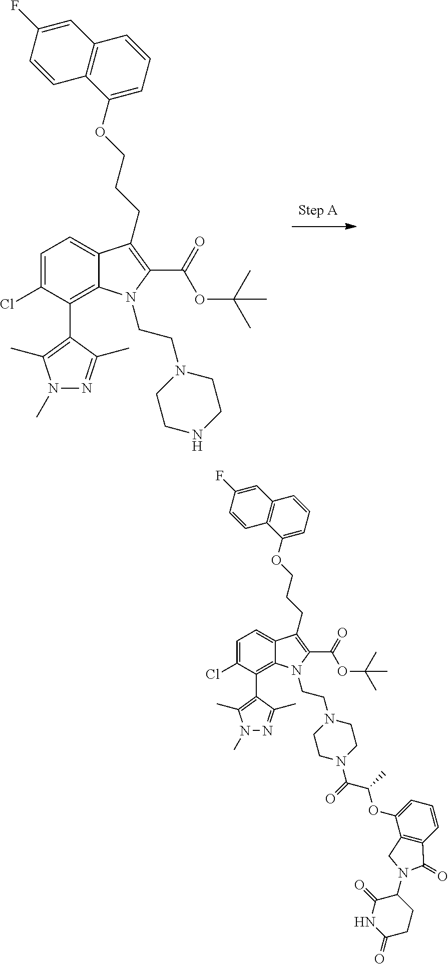

An equimolar mixture of (S)-2-((2-((S)-2,6-dioxopiperidin-3-yl)-1-oxoisoindolin-4-yl)oxy)propanoic acid and (S)-2-((2-((R)-2,6-dioxopiperidin-3-yl)-1-oxoisoindolin-4-yl)oxy)propanoic acid (9.8 mg, 0.029 mmol) was dissolved in dry DMF (0.979 mL) and DIPEA (0.013 mL, 0.073 mmol), followed by HATU (9.8 mg, 0.026 mmol) was added and the solution was stirred for 15 min in room temperature under argon. To it, tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (16.5 mg, 0.024 mmol) was added and the reaction (monitored with LCMS) was continued under argon at room temperature for 15 min. After complete consumption of the starting material, the mixture was diluted with DCM and washed with water and brine. Organic layer was dried over anhydrous MgSO₄, filtrated and concentrated under reduced pressure. Crude tert-butyl 6-chloro-1-(2-{4-[(2S)-2-{[2-(2,6-dioxopiperidin-3-yl)-1-oxo-2,3-dihydro-1H-isoindol-4-yl]oxy}propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate (25.0 mg) was obtained as yellow oil and used in the next step without further purification.

LCMS (ESI+): m/z 988.2 [M+H]⁺.

### Step B

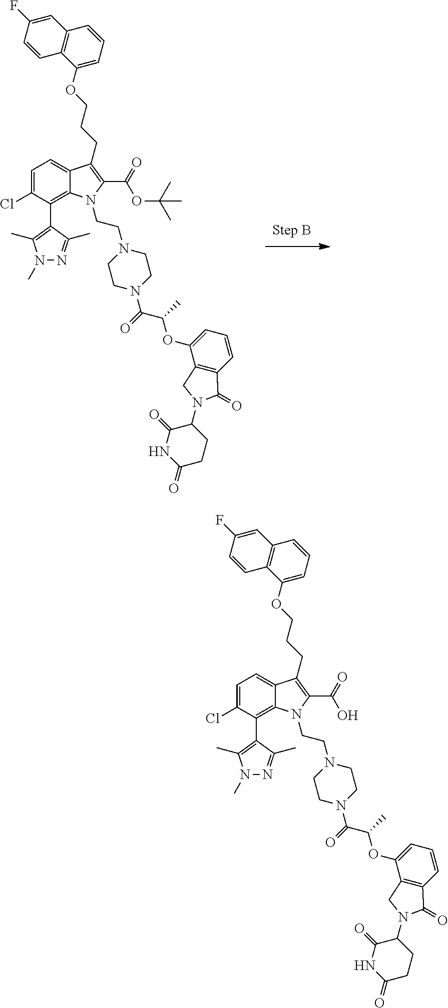 [SMILES: Cc1nn(C)c(C)c1-c1c(Cl)ccc2c(CCCOc3cccc4cc(F)ccc34)c(C(=O)OC(C)(C)C)n(CCN3CCN(C(=O)[C@H](C)Oc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)[C@H](C)Oc4cccc5c4CN(C4CCC(=O)NC4=O)C5=O)CC3)c12]

Crude tert-butyl 6-chloro-1-(2-{4-[(2S)-2-{[2-(2,6-dioxopiperidin-3-yl)-1-oxo-2,3-dihydro-1H-isoindol-4-yl]oxy}propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate (25.0 mg) was dissolved in dry DCM (0.194 mL) under argon and TFA (0.194 mL, 2.529 mmol) was added. Reaction (monitored with LCMS) was stirred at room temperature under argon. After complete consumption of the starting material the mixture was concentrated under reduced pressure to dryness. The resulting residue was dissolved in DMSO, passed through a syringe filter and purified with preparative HPLC (C18, H₂O:MeCN+0.1% FA) to give 6-chloro-1-(2-(4-((2S)-2-((2-(2,6-dioxopiperidin-3-yl)-1-oxoisoindolin-4-yl)oxy)propanoyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (11.4 mg, 0.012 mmol, 50.0% over 2 steps) as white solid.

LCMS (ESI+): m/z 932.2 [M+H]⁺.

¹H NMR (500 MHz, DMSO, 353 K) δ 10.64 (s, 1H), 8.23 (ddd, J=9.2, 6.0, 1.4 Hz, 1H), 7.71 (d, J=8.5 Hz, 1H), 7.58 (dd, J=10.4, 2.7 Hz, 1H), 7.47-7.38 (m, 3H), 7.36-7.28 (m, 2H), 7.21 (d, J=8.5 Hz, 1H), 7.09-7.02 (m, 1H), 6.91-6.82 (m, 1H), 5.37-5.27 (m, 1H), 5.05 (ddd, J=13.0, 5.3, 4.0 Hz, 1H), 4.38 (d, J=17.2 Hz, 1H), 4.34-4.29 (m, 1H), 4.29-4.21 (m, 3H), 4.21-4.13 (m, 1H), 3.77-3.70 (m, 3H), 3.40 (bs, 4H), 3.28 (dd, J=8.3, 6.7 Hz, 2H), 2.92-2.83 (m, 1H), 2.66-2.59 (m, 1H), 2.47-2.40 (m, 1H), 2.27-2.20 (m, 2H), 2.13-2.01 (m, 7H), 2.01-1.97 (m, 3H), 1.90-1.84 (m, 3H), 1.45 (dd, J=6.6, 1.3 Hz, 3H).

### Example 84. 6-Chloro-1-(2-{4-[(2R)-2-({2-[(3R)-2,6-dioxopiperidin-3-yl]-1,3-dioxo-2,3-dihydro-1H-isoindol-4-yl}oxy)propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylic acid (316)

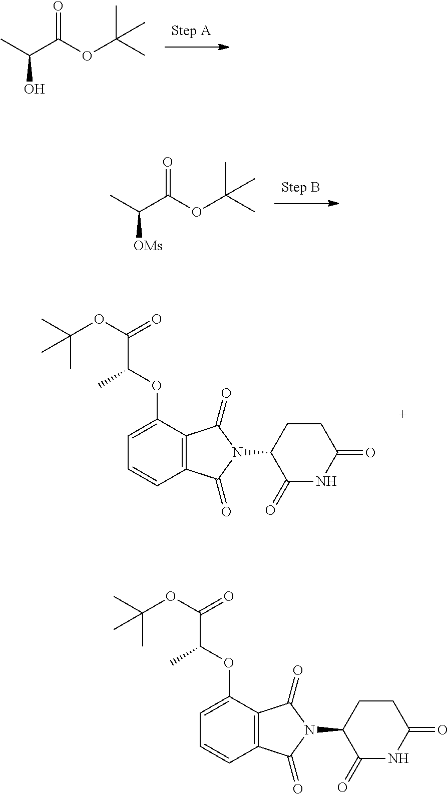

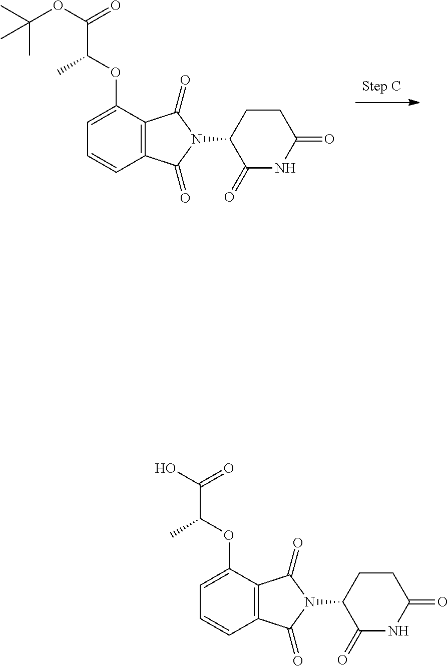 [SMILES: C[C@@H](Oc1cccc2c1C(=O)N([C@@H]1CCC(=O)NC1=O)C2=O)C(=O)OC(C)(C)C ; C[C@@H](Oc1cccc2c1C(=O)N([C@@H]1CCC(=O)NC1=O)C2=O)C(=O)O]

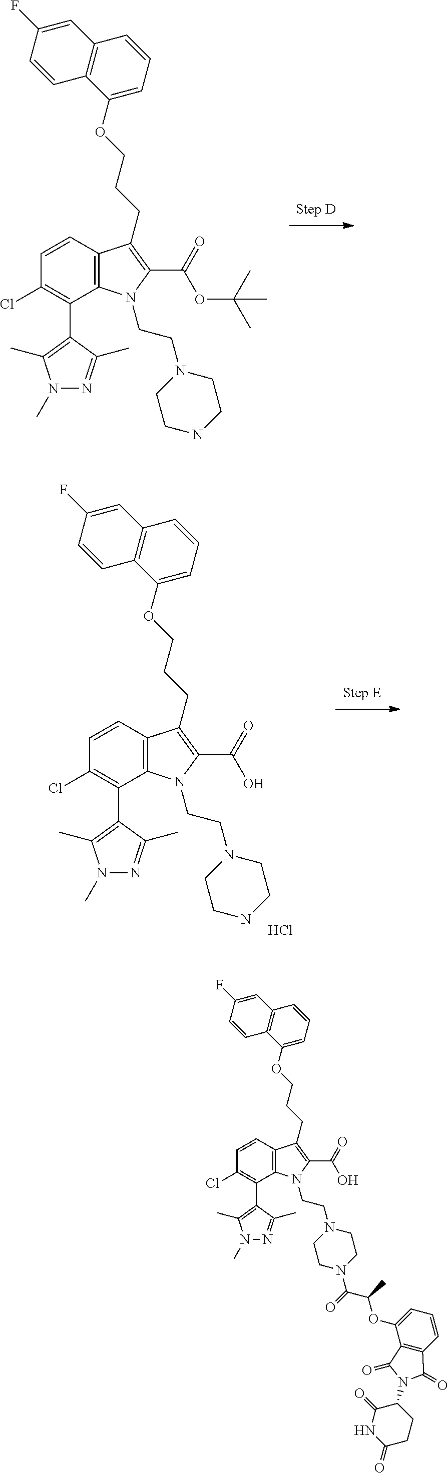

### Step A

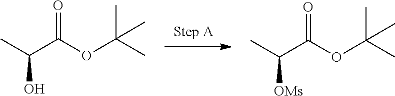

To a stirred solution of tert-butyl (S)-2-hydroxypropanoate (500 mg, 3.42 mmol) in DCM (15 mL) was added MsCl (0.530 mL, 6.5 mmol) followed by Et₃N (1.04 mL, 10.27 mmol) at 0° C. under nitrogen and the reaction mixture was allowed to stir at same temperature for 1 h. After complete consumption of the starting material (monitored by TLC) the reaction mixture was diluted with EtOAc, washed successively with water and brine, the organic layer was dried over Na₂SO₄ and evaporated under reduced pressure to get the crude compound, which was then purified by column chromatography (SiO₂, 10-20% EtOAc in hexane) to obtain tert-butyl (S)-2-((methylsulfonyl)oxy)propanoate (500 mg, 2.23 mmol, 65%) as white solid.

### Step B

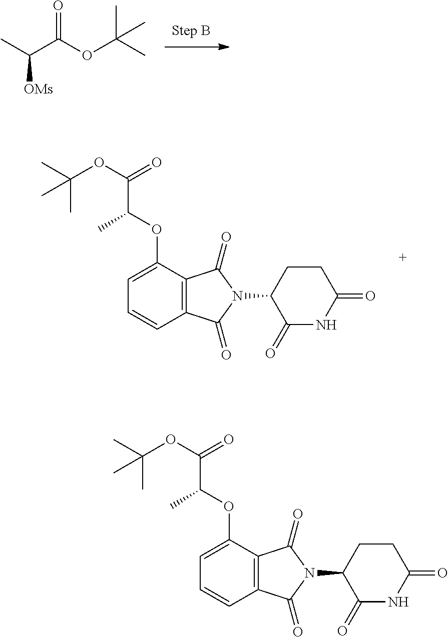 [SMILES: C[C@H](OS(C)(=O)=O)C(=O)OC(C)(C)C ; C[C@@H](Oc1cccc2c1C(=O)N([C@@H]1CCC(=O)NC1=O)C2=O)C(=O)OC(C)(C)C ; C[C@@H](Oc1cccc2c1C(=O)N([C@H]1CCC(=O)NC1=O)C2=O)C(=O)OC(C)(C)C]

To a well stirred solution of tert-butyl (S)-2-((methylsulfonyl)oxy)propanoate (250 mg, 1.16 mmol) in DMF (8 mL) was added KHCO₃ (0.70 mg, 7 mmol) and 2-(2,6-dioxopiperidin-3-yl)-4-hydroxyisoindoline-1,3-dione (367 mg, 1.33 mmol) successively at room temperature under nitrogen. The reaction mixture was allowed to stir at 60° C. for 3 h. After complete consumption of the starting material (monitored by LCMS) the reaction mixture was diluted with EtOAc, washed successively with water and brine. The organic layer was dried over Na₂SO₄ and evaporated under reduced pressure to get the crude compound, which was then purified by column chromatography (SiO₂, 30-40% EtOAc in DCM) to get 250 mg tert-butyl (2R)-2-((2-(2,6-dioxopiperidin-3-yl)-1,3-dioxoisoindolin-4-yl)oxy)propanoate. This mixture of enantiomers was separated by preparative chiral HPLC to obtain tert-butyl (R)-2-((2-((R)-2,6-dioxopiperidin-3-yl)-1,3-dioxoisoindolin-4-yl)oxy)propanoate (100 mg, 0.25 mmol, 22.7%)

LCMS (ESI+): m/z 401.2 [M+H]⁺

and tert-butyl (R)-2-((2-((S)-2,6-dioxopiperidin-3-yl)-1,3-dioxoisoindolin-4-yl)oxy)propanoate (120 mg, 0.3 mmol, 26.7%)

LCMS (ESI+): m/z 401.2 [M+H]⁺

Absolute stereochemistry of compounds is arbitrarily assigned.

### Step C

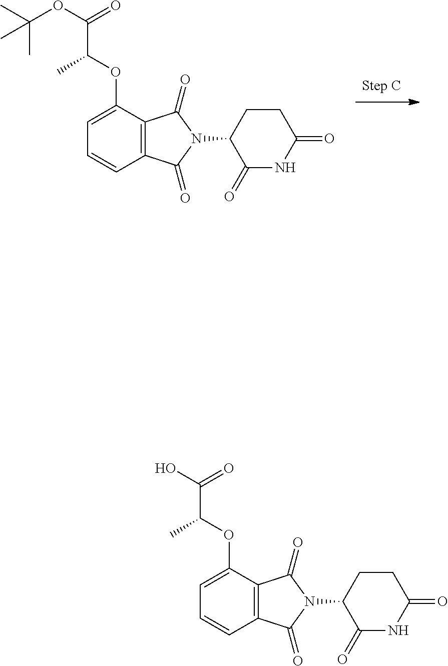 [SMILES: C[C@@H](Oc1cccc2c1C(=O)N([C@@H]1CCC(=O)NC1=O)C2=O)C(=O)OC(C)(C)C ; C[C@@H](Oc1cccc2c1C(=O)N([C@@H]1CCC(=O)NC1=O)C2=O)C(=O)O]

To a stirred solution of tert-butyl (R)-2-((2-((R)-2,6-dioxopiperidin-3-yl)-1,3-dioxoisoindolin-4-yl)oxy)propanoate (100 mg, 0.25 mmol) in DCM (3 mL) was added TFA (3 mL) drop wise at 0° C. under nitrogen and the reaction mixture was allowed to stir at room temperature for 16 h. After consuming of starting material (monitored by LCMS) the volatiles were evaporated under reduced pressure to get the crude compound, which was then purified by triturating it by Et₂O and pentane to afford (R)-2-((2-((R)-2,6-dioxopiperidin-3-yl)-1,3-dioxoisoindolin-4-yl)oxy)propanoic acid (41 mg, 0.115 mmol, 47.6%) as brown solid.

LCMS (ESI+): m/z 347.2 [M+H]⁺

### Step D

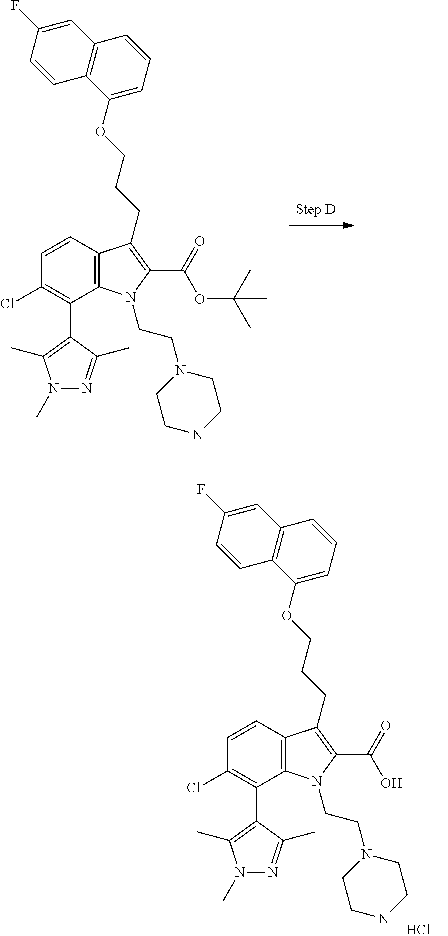

To a solution of tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (106.0 mg, 0.157 mmol) in THE (1.0 mL) was added 4M HCl in dioxane (2.1 mL, 61.053 mmol). The mixture was stirred for 18 h at room temperature and next 18 h at 40° C. The crude was concentrated in vacuo and dissolved in 1M HCl to obtain 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylic acid hydrochloride (107.0 mg, 0.163 mmol, quantitative) as a white solid.

LCMS (ESI+): m/z 618.3 [M+H]⁺

### Step E

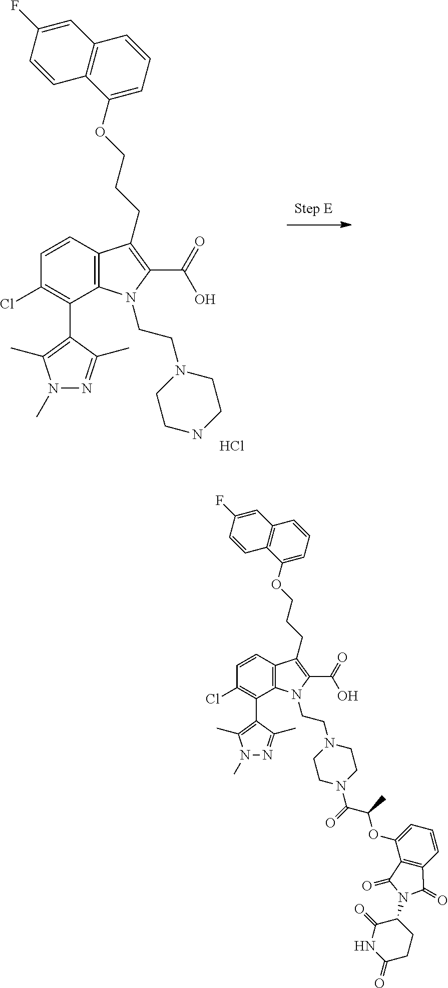

To the mixture (R)-2-((2-((R)-2,6-dioxopiperidin-3-yl)-1,3-dioxoisoindolin-4-yl)oxy)propanoic acid (11.9 mg, 0.034 mmol) and HATU (12.2 mg, 0.032 mmol) in dry DMF (2.4 mL) was added DIPEA (0.020 mL, 0.115 mmol) and was stirred for 1 h at RT. To the mixture was added a solution of 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylic acid hydrochloride (15 mg, 0.023 mmol). The reaction was stirred next 30 min at RT. The crude was purified by preparative HPLC (C18, H₂O:MeCN+0.1% FA) to obtain 6-chloro-1-(2-{4-[(2R)-2-({2-[(3R)-2,6-dioxopiperidin-3-yl]-1,3-dioxo-2,3-dihydro-1H-isoindol-4-yl}oxy)propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylic acid (5.4 mg, 0.006 mmol, 24.9%) as an off white solid.

LCMS (ESI+): m/z 946.35 [M+H]⁺

¹H NMR (500 MHz, DMSO, 353 K) δ 10.79 (s, 1H), 8.24 (dd, J=9.2, 5.9 Hz, 1H), 7.76 (dd, J=8.5, 7.3 Hz, 1H), 7.67 (d, J=8.5 Hz, 1H), 7.58 (dd, J=10.4, 2.6 Hz, 1H), 7.45 (d, J=7.1 Hz, 1H), 7.43-7.39 (m, 2H), 7.32 (td, J=8.9, 2.6 Hz, 1H), 7.23 (d, J=8.5 Hz, 1H), 7.19 (d, J=8.5 Hz, 1H), 6.87 (dd, J=6.0, 2.7 Hz, 1H), 5.47-5.42 (m, 1H), 5.04 (dd, J=12.5, 5.5 Hz, 1H), 4.31-4.22 (m, 3H), 4.21-4.14 (m, 1H), 3.74 (d, J=5.3 Hz, 3H), 3.42 (s, 4H), 3.26 (t, J=7.5 Hz, 2H), 2.87 (ddd, J=16.8, 13.4, 5.2 Hz, 1H), 2.65-2.53 (m, 2H), 2.23 (p, J=6.6 Hz, 2H), 2.13-2.05 (m, 7H), 2.00-1.98 (m, 3H), 1.87 (s, 3H), 1.49 (dd, J=6.5, 1.2 Hz, 3H).

### Example 85. 6-Chloro-1-(2-{4-[(2R)-2-({2-[(3S)-2,6-dioxopiperidin-3-yl]-1,3-dioxo-2,3-dihydro-1H-isoindol-4-yl}oxy)propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylic acid (317)

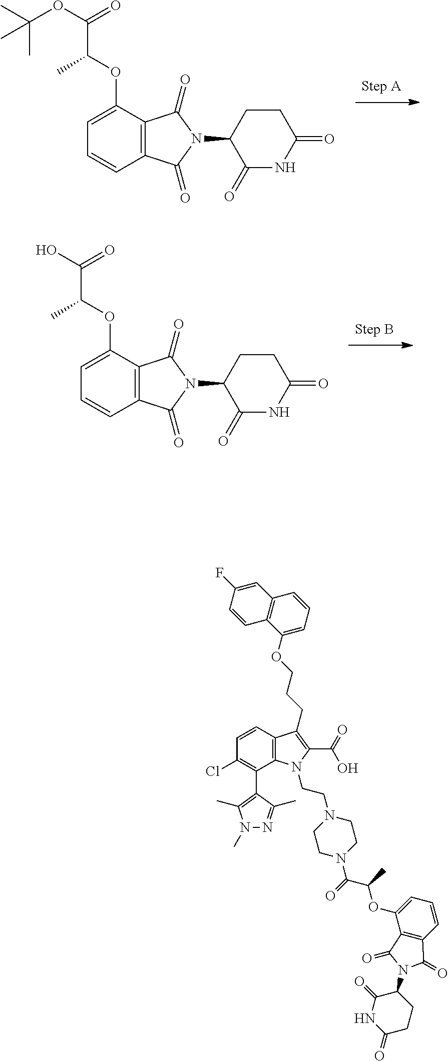 [SMILES: C[C@@H](Oc1cccc2c1C(=O)N([C@H]1CCC(=O)NC1=O)C2=O)C(=O)OC(C)(C)C ; C[C@@H](Oc1cccc2c1C(=O)N([C@H]1CCC(=O)NC1=O)C2=O)C(=O)O ; Cc1nn(C)c(C)c1-c1c(Cl)ccc2c(CCCOc3cccc4cc(F)ccc34)c(C(=O)O)n(CCN3CCN(C(=O)[C@@H](C)Oc4cccc5c4C(=O)N([C@H]4CCC(=O)NC4=O)C5=O)CC3)c12]

### Step A

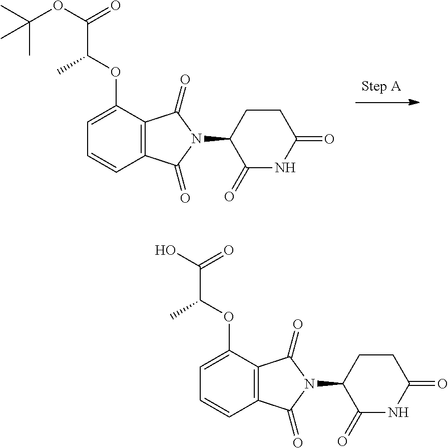 [SMILES: C[C@@H](Oc1cccc2c1C(=O)N([C@H]1CCC(=O)NC1=O)C2=O)C(=O)OC(C)(C)C ; C[C@@H](Oc1cccc2c1C(=O)N([C@H]1CCC(=O)NC1=O)C2=O)C(=O)O]

To a stirred solution of tert-butyl (R)-2-((2-((S)-2,6-dioxopiperidin-3-yl)-1,3-dioxoisoindolin-4-yl)oxy)propanoate (120 mg, 0.3 mmol) in DCM (3 mL) was added TFA (3 mL) drop wise at 0° C. under nitrogen and the reaction mixture was allowed to stir at room temperature for 16 h. After consuming of starting material (monitored by LCMS) the volatiles were evaporated under reduced pressure to get the crude compound which was then purified by triturating it by Et₂O and pentane to afford (R)-2-((2-((S)-2,6-dioxopiperidin-3-yl)-1,3-dioxoisoindolin-4-yl)oxy)propanoic acid (40 mg, 0.115 mmol, 38.7%) of as light brown solid.

LCMS (ESI+): m/z 347.15 [M+H]⁺

### Step B

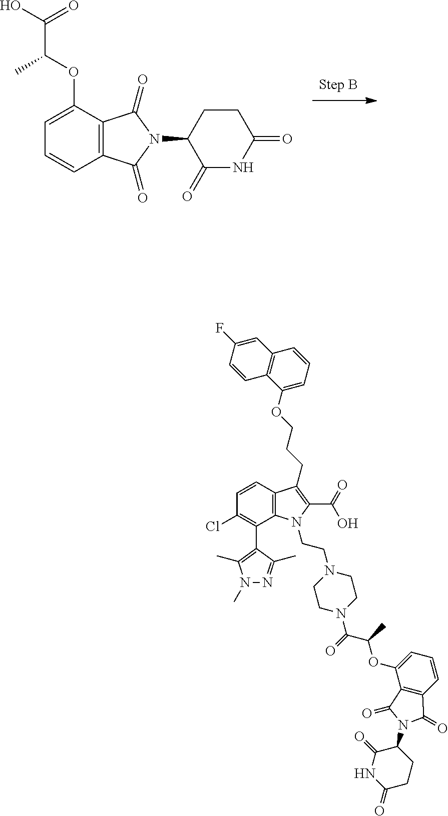 [SMILES: C[C@@H](Oc1cccc2c1C(=O)N([C@H]1CCC(=O)NC1=O)C2=O)C(=O)O ; Cc1nn(C)c(C)c1-c1c(Cl)ccc2c(CCCOc3cccc4cc(F)ccc34)c(C(=O)O)n(CCN3CCN(C(=O)[C@@H](C)Oc4cccc5c4C(=O)N([C@H]4CCC(=O)NC4=O)C5=O)CC3)c12]

To the mixture (R)-2-((2-((S)-2,6-dioxopiperidin-3-yl)-1,3-dioxoisoindolin-4-yl)oxy)propanoic acid (11.9 mg, 0.034 mmol) and HATU (12.2 mg, 0.032 mmol) in dry DMF (2.4 mL) was added DIPEA (0.020 mL, 0.115 mmol) and was stirred for 1 h at RT. To the mixture was added a solution of 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylic acid hydrochloride (15 mg, 0.23 mmol). The reaction was stirred next 30 min at RT. The crude was purified by preparative HPLC (C18, H₂O:MeCN+0.1% FA) to obtain 6-chloro-1-(2-{4-[(2R)-2-({2-[(3S)-2,6-dioxopiperidin-3-yl]-1,3-dioxo-2,3-dihydro-1H-isoindol-4-yl}oxy)propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylic acid (6.5 mg, 0.007 mmol, 30.0%) as an off white solid.

LCMS (ESI+): m/z 946.25 [M+H]⁺

¹H NMR (500 MHz, DMSO, 353 K) δ 10.79 (s, 1H), 8.24 (dd, J=9.3, 5.9 Hz, 1H), 7.76 (dd, J=8.5, 7.3 Hz, 1H), 7.69 (d, J=8.5 Hz, 1H), 7.58 (dd, J=10.4, 2.6 Hz, 1H), 7.45 (dt, J=7.3, 0.7 Hz, 1H), 7.43-7.39 (m, 2H), 7.32 (td, J=8.9, 2.6 Hz, 1H), 7.21 (dd, J=14.8, 8.5 Hz, 2H), 6.87 (dd, J=6.0, 2.6 Hz, 1H), 5.47-5.41 (m, 1H), 5.04 (dd, J=12.4, 5.5 Hz, 1H), 4.30-4.22 (m, 3H), 4.20-4.14 (m, 1H), 3.74 (d, J=4.2 Hz, 3H), 3.42 (s, 4H), 3.27 (t, J=7.5 Hz, 2H), 2.87 (ddd, J=17.0, 13.4, 5.2 Hz, 1H), 2.65-2.53 (m, 2H), 2.23 (p, J=6.5 Hz, 2H), 2.14-2.03 (m, 7H), 2.00 (d, J=3.4 Hz, 3H), 1.87 (s, 3H), 1.49 (d, J=6.5 Hz, 3H).

### Example 86. 6-Chloro-1-(2-{4-[(2R)-2-{[2-(2,6-dioxopiperidin-3-yl)-1,3-dioxo-2,3-dihydro-1H-isoindol-4-yl]oxy}propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylic acid (315)

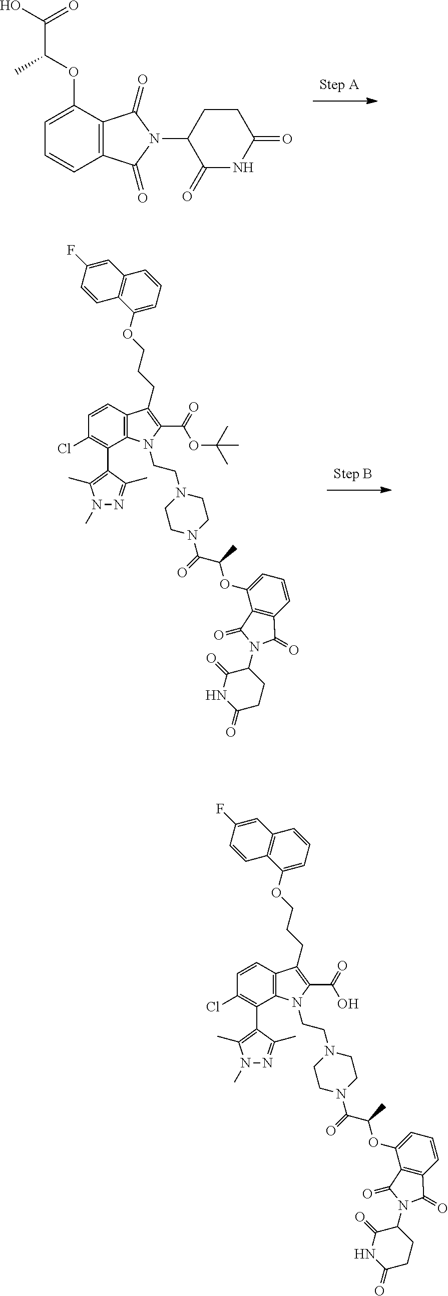 [SMILES: C[C@@H](Oc1cccc2c1C(=O)N(C1CCC(=O)NC1=O)C2=O)C(=O)O ; Cc1nn(C)c(C)c1-c1c(Cl)ccc2c(CCCOc3cccc4cc(F)ccc34)c(C(=O)OC(C)(C)C)n(CCN3CCN(C(=O)[C@@H](C)Oc4cccc5c4C(=O)N(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)[C@@H](C)Oc4cccc5c4C(=O)N(C4CCC(=O)NC4=O)C5=O)CC3)c12]

### Step A

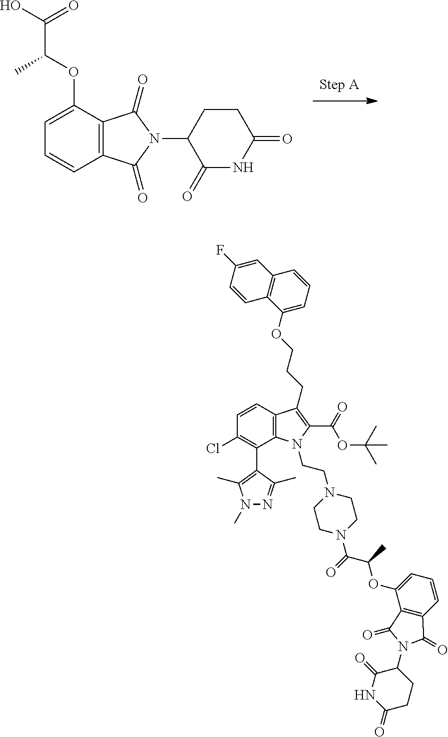 [SMILES: C[C@@H](Oc1cccc2c1C(=O)N(C1CCC(=O)NC1=O)C2=O)C(=O)O ; Cc1nn(C)c(C)c1-c1c(Cl)ccc2c(CCCOc3cccc4cc(F)ccc34)c(C(=O)OC(C)(C)C)n(CCN3CCN(C(=O)[C@@H](C)Oc4cccc5c4C(=O)N(C4CCC(=O)NC4=O)C5=O)CC3)c12]

To a solution of tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (25.0 mg, 0.037 mmol), (2R)-2-({2-[(3S)-2,6-dioxopiperidin-3-yl]-1,3-dioxo-2,3-dihydro-1H-isoindol-4-yl}oxy)propanoic acid (7.7 mg, 0.022 mmol), (2R)-2-({2-[(3R)-2,6-dioxopiperidin-3-yl]-1,3-dioxo-2,3-dihydro-1H-isoindol-4-yl}oxy)propanoic acid (7.7 mg, 0.022 mmol) and HATU (28.2 mg, 0.074 mmol) in dry DMF (2.0 mL) was added DIPEA (0.019 mL, 0.111 mmol). The mixture was stirred at room temperature for 30 min.

The crude was diluted with DCM and washed with brine. The organic layer was dried over MgSO₄, filtered and concentrated in vacuo to get 59 mg crude with tert-butyl 6-chloro-1-(2-{4-[(2R)-2-{[2-(2,6-dioxopiperidin-3-yl)-1,3-dioxo-2,3-dihydro-1H-isoindol-4-yl]oxy}propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate as orange oil which was used in the next step without further purification.

LCMS (ESI+): m/z 618.3 [M+H]⁺

### Step B

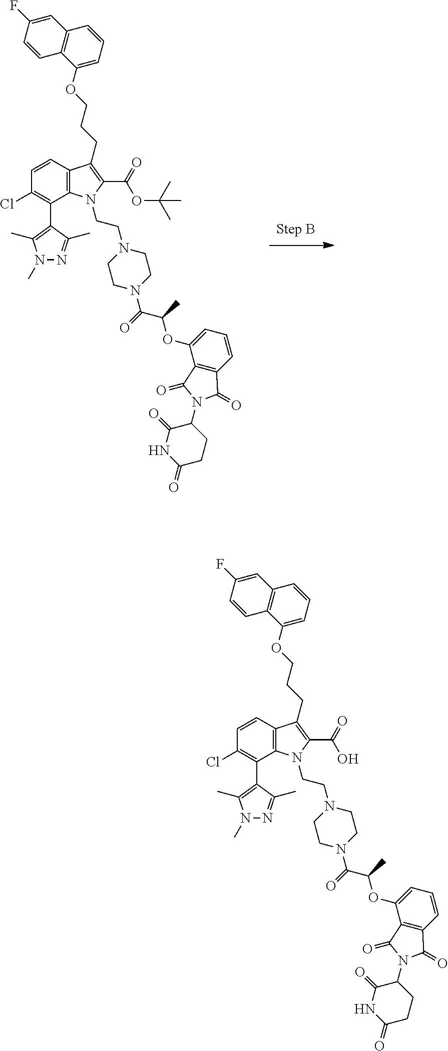 [SMILES: Cc1nn(C)c(C)c1-c1c(Cl)ccc2c(CCCOc3cccc4cc(F)ccc34)c(C(=O)OC(C)(C)C)n(CCN3CCN(C(=O)[C@@H](C)Oc4cccc5c4C(=O)N(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)[C@@H](C)Oc4cccc5c4C(=O)N(C4CCC(=O)NC4=O)C5=O)CC3)c12]

To a solution of tert-butyl 6-chloro-1-(2-{4-[(2R)-2-{[2-(2,6-dioxopiperidin-3-yl)-1,3-dioxo-2,3-dihydro-1H-isoindol-4-yl]oxy}propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate (59 mg, crude) in DCM (1.0 mL) was added TFA (1.0 mL). The mixture was stirred for 18 h at RT. The crude was concentrated in vacuo and purified by preparative HPLC (C18, H₂O:MeCN+0.1% FA) to obtain 6-chloro-1-(2-{4-[(2R)-2-{[2-(2,6-dioxopiperidin-3-yl)-1,3-dioxo-2,3-dihydro-1H-isoindol-4-yl]oxy}propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylic acid (22.7 mg, 0.024 mmol, 64.9% over two steps) as a white solid.

LCMS (ESI+): m/z 946.14 [M+H]⁺

¹H NMR (500 MHz, DMSO, 353 K) δ 10.81 (s, 1H), 8.23 (dd, J=9.2, 5.9 Hz, 1H), 7.76 (dd, J=8.5, 7.3 Hz, 1H), 7.71 (d, J=8.5 Hz, 1H), 7.59 (dd, J=10.4, 2.7 Hz, 1H), 7.47-7.39 (m, 3H), 7.35-7.30 (m, 1H), 7.22 (dd, J=8.5, 3.5 Hz, 2H), 6.87 (dd, J=6.0, 2.6 Hz, 1H), 5.48-5.41 (m, 1H), 5.04 (dd, J=12.5, 5.5 Hz, 1H), 4.30-4.22 (m, 3H), 4.17 (q, J=7.2 Hz, 1H), 3.74 (d, J=4.5 Hz, 3H), 3.42 (s, 4H), 3.32-3.25 (m, 2H), 2.87 (ddd, J=17.6, 13.3, 5.3 Hz, 1H), 2.65-2.52 (m, 2H), 2.27-2.20 (m, 2H), 2.12-2.03 (m, 7H), 2.02-1.98 (m, 3H), 1.87 (s, 3H), 1.49 (d, J=6.5 Hz, 3H).

### Example 87. 6-Chloro-1-(2-(4-((S)-2-((2-((R)-2,6-dioxopiperidin-3-yl)-1,3-dioxoisoindolin-4-yl)oxy)propanoyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (320)

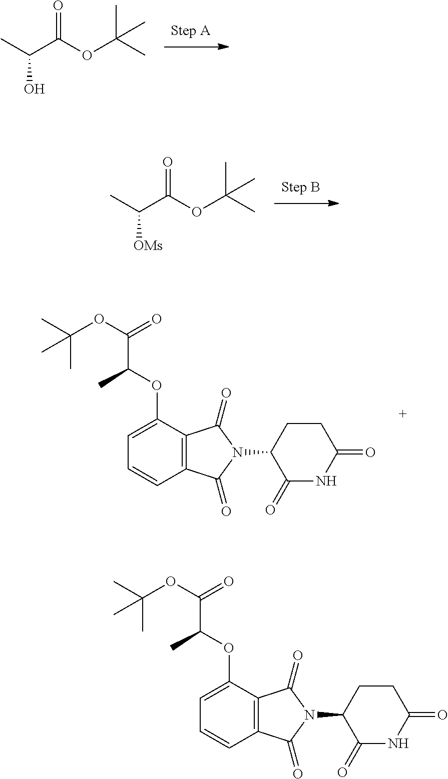

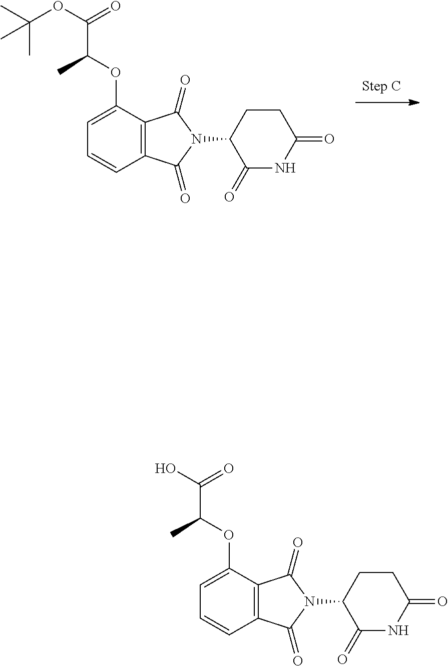 [SMILES: C[C@H](Oc1cccc2c1C(=O)N([C@@H]1CCC(=O)NC1=O)C2=O)C(=O)OC(C)(C)C ; C[C@H](Oc1cccc2c1C(=O)N([C@@H]1CCC(=O)NC1=O)C2=O)C(=O)O]

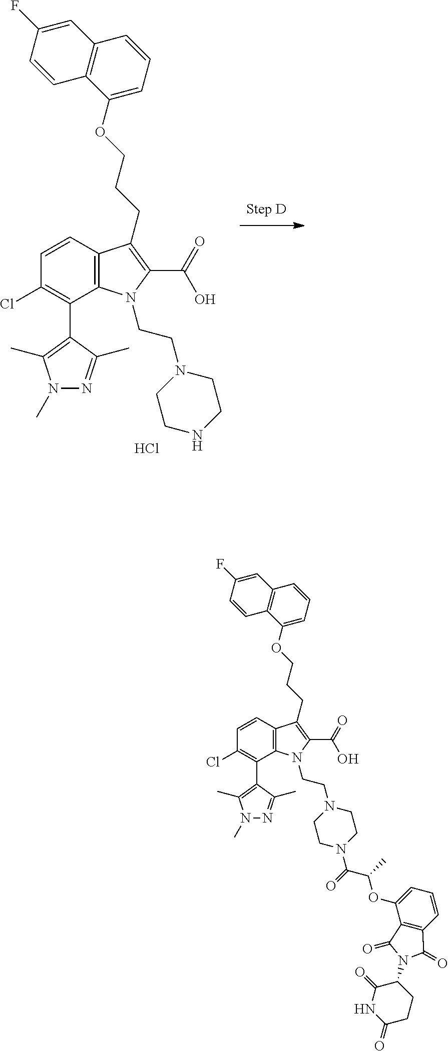 [SMILES: Cc1nn(C)c(C)c1-c1c(Cl)ccc2c(CCCOc3cccc4cc(F)ccc34)c(C(=O)O)n(CCN3CCNCC3)c12 ; Cl ; Cc1nn(C)c(C)c1-c1c(Cl)ccc2c(CCCOc3cccc4cc(F)ccc34)c(C(=O)O)n(CCN3CCN(C(=O)[C@H](C)Oc4cccc5c4C(=O)N([C@@H]4CCC(=O)NC4=O)C5=O)CC3)c12]

### Step A

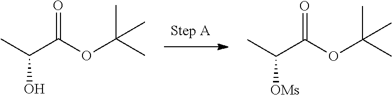

To a solution of tert-butyl (R)-2-hydroxypropanoate (500 mg, 3.42 mmol) in dichloromethane (15 mL) was added mesyl chloride (0.530 mL, 6.5 mmol) followed by triethyl amine (1.04 mL, 10.27 mmol) at 0° C. under nitrogen and the reaction mixture was allowed to stir at same temperature for 1 h. After complete consumption of the starting material (monitored by TLC) the reaction mixture was diluted with EtOAc and washed successively with water and brine. The organic layer was dried over Na₂SO₄ and evaporated under reduced pressure to get the crude compound, which was then purified with column chromatography (SiO₂, 10-20% EtOAc in hexane) to get tert-butyl (R)-2-((methylsulfonyl)oxy)propanoate (500 mg 2.23 mmol, 65%) as white solid.

### Step B

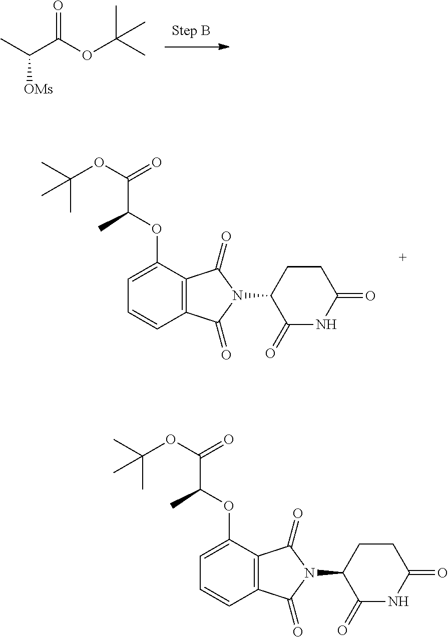 [SMILES: C[C@@H](OS(C)(=O)=O)C(=O)OC(C)(C)C ; C[C@H](Oc1cccc2c1C(=O)N([C@@H]1CCC(=O)NC1=O)C2=O)C(=O)OC(C)(C)C ; C[C@H](Oc1cccc2c1C(=O)N([C@H]1CCC(=O)NC1=O)C2=O)C(=O)OC(C)(C)C]

To a well stirred solution of tert-butyl (R)-2-((methylsulfonyl)oxy)propanoate (250 mg, 1.16 mmol) in DMF (8 mL) was added KHCO₃ (0.70 mg, 7.0 mmol) and 2-(2,6-dioxopiperidin-3-yl)-4-hydroxyisoindoline-1,3-dione (0.367 g, 1.33 mmol) at room temperature under nitrogen. The reaction mixture was allowed to stir at 60° C. for 3 h. After complete consumption of the starting material the reaction mixture was diluted with EtOAc and washed successively with water and brine. The organic layer was dried over Na₂SO₄ and evaporated under reduced pressure to get the crude compound, which was then purified by column chromatography (SiO₂, 30-40% EtOAc in DCM) to get 250 mg of crude tert-butyl (2S)-2-((2-(2,6-dioxopiperidin-3-yl)-1,3-dioxoisoindolin-4-yl)oxy)propanoate. This mixture of enantiomers was separated by preparative chiral HPLC to obtain tert-butyl (S)-2-((2-((S)-2,6-dioxopiperidin-3-yl)-1,3-dioxoisoindolin-4-yl)oxy)propanoate (80 mg, 0.2 mmol, 17.2%) as off white solid

LCMS (ESI−): m/z 401.2 [M−H]⁻

and tert-butyl (S)-2-((2-((R)-2,6-dioxopiperidin-3-yl)-1,3-dioxoisoindolin-4-yl)oxy)propanoate (100 mg, 0.248 mmol, 22.72%) of off white solid.

LCMS (ESI−): m/z 401.2 [M−H]⁻

Absolute stereochemistry of compounds is arbitrarily assigned.

### Step C

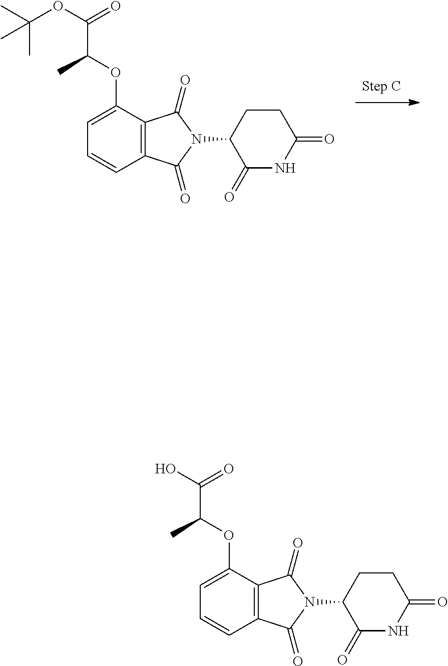 [SMILES: C[C@H](Oc1cccc2c1C(=O)N([C@@H]1CCC(=O)NC1=O)C2=O)C(=O)OC(C)(C)C ; C[C@H](Oc1cccc2c1C(=O)N([C@@H]1CCC(=O)NC1=O)C2=O)C(=O)O]

To a stirred solution of tert-butyl (S)-2-((2-((R)-2,6-dioxopiperidin-3-yl)-1,3-dioxoisoindolin-4-yl)oxy)propanoate (100 mg, 0.25 mmol) in DCM (3 mL) was added TFA (3 mL) drop wise at 0° C. under nitrogen and the reaction mixture was allowed to stir at room temperature for 16 h. After consuming of starting material the volatiles were evaporated under reduced pressure to get the crude compound which was then purified by triturating it by diethyl ether and pentane to afford (S)-2-((2-((R)-2,6-dioxopiperidin-3-yl)-1,3-dioxoisoindolin-4-yl)oxy)propanoate (60 mg, 0.1734 mmol, 69%) of as brown solid.

LCMS (ESI+): m/z 347.3 [M+H]⁺

### Step D

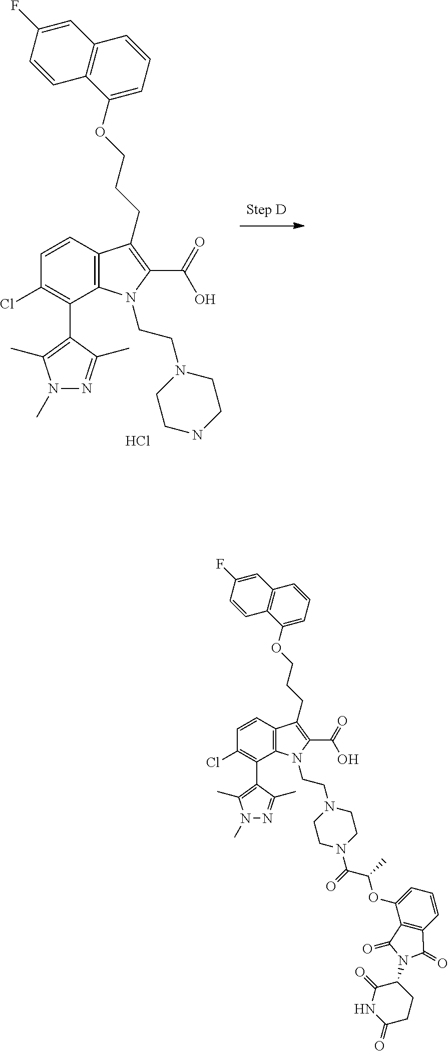 [SMILES: Cc1nn(C)c(C)c1-c1c(Cl)ccc2c(CCCOc3cccc4cc(F)ccc34)c(C(=O)O)n(CCN3CCNCC3)c12 ; Cl ; Cc1nn(C)c(C)c1-c1c(Cl)ccc2c(CCCOc3cccc4cc(F)ccc34)c(C(=O)O)n(CCN3CCN(C(=O)[C@H](C)Oc4cccc5c4C(=O)N([C@@H]4CCC(=O)NC4=O)C5=O)CC3)c12]

(2S)-2-({2-[(3R)-2,6-Dioxopiperidin-3-yl]-1,3-dioxo-2,3-dihydro-1H-isoindol-4-yl}oxy)propanoic acid (10.1 mg, 0.029 mmol) and HATU (10.3 mg, 0.027 mmol) were dissolved in dry DMF (2.4 mL) under argon atmosphere and DIPEA (0.010 mL, 0.058 mmol) was added. Reaction was stirred in room temperature for 1 h. 6-Chloro-3-(3-((6-fluoronaphthalen-1-yl)oxy)propyl)-1-(2-(piperazin-1-yl)ethyl)-7-(1,3,5-trimethyl-1H-pyrazol-4-yl)-1H-indole-2-carboxylic acid hydrochloride (12.7 mg, 0.019 mmol) was dissolved in DMSO (0.600 mL) with DIPEA (0.010 mL, 0.058 mmol) was slowly added dropwise to the reaction which was then stirred under argon atmosphere in room temperature for 15 min. After complete consumption of the starting material the solution was diluted with DMSO to 5 mL and crude product was purified with preparative HPLC (H₂O:MeCN+0.1% FA) to give 6-chloro-1-(2-(4-((S)-2-((2-((R)-2,6-dioxopiperidin-3-yl)-1,3-dioxoisoindolin-4-yl)oxy)propanoyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (6.7 mg, 0.007 mmol, 36.2%) as white powder.

LCMS (ESI+): m/z 946 [M+H]⁺

¹H NMR (500 MHz, DMSO, 353 K) δ 10.77 (s, 1H), 8.23 (dd, J=9.1, 5.7 Hz, 1H), 7.76 (dd, J=8.5, 7.2 Hz, 1H), 7.71 (d, J=8.5 Hz, 1H), 7.58 (dd, J=10.4, 2.6 Hz, 1H), 7.48-7.38 (m, 3H), 7.32 (td, J=8.9, 2.7 Hz, 1H), 7.22 (dd, J=8.5, 7.0 Hz, 2H), 6.87 (dd, J=6.0, 2.6 Hz, 1H), 5.51-5.38 (m, 1H), 5.04 (dd, J=12.5, 5.5 Hz, 1H), 4.34-4.09 (m, 4H), 3.74 (d, J=4.0 Hz, 3H), 3.42 (bs, 4H), 3.33-3.23 (m, 2H), 2.87 (ddd, J=16.9, 13.4, 5.3 Hz, 1H), 2.65-2.52 (m, 2H), 2.28-2.19 (m, 2H), 2.16-2.02 (m, 7H), 2.02-1.97 (m, 3H), 1.87 (d, J=1.0 Hz, 3H), 1.49 (d, J=6.6 Hz, 3H).

### Example 88. 6-Chloro-1-(2-(4-((S)-2-((2-((S)-2,6-dioxopiperidin-3-yl)-1,3-dioxoisoindolin-4-yl)oxy)propanoyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (319)

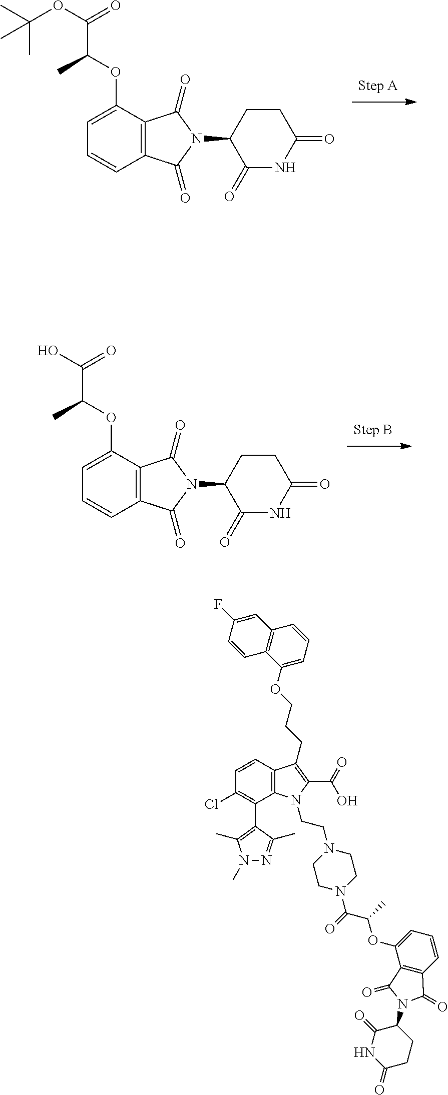 [SMILES: C[C@H](Oc1cccc2c1C(=O)N([C@H]1CCC(=O)NC1=O)C2=O)C(=O)OC(C)(C)C ; C[C@H](Oc1cccc2c1C(=O)N([C@H]1CCC(=O)NC1=O)C2=O)C(=O)O ; Cc1nn(C)c(C)c1-c1c(Cl)ccc2c(CCCOc3cccc4cc(F)ccc34)c(C(=O)O)n(CCN3CCN(C(=O)[C@H](C)Oc4cccc5c4C(=O)N([C@H]4CCC(=O)NC4=O)C5=O)CC3)c12]

### Step A

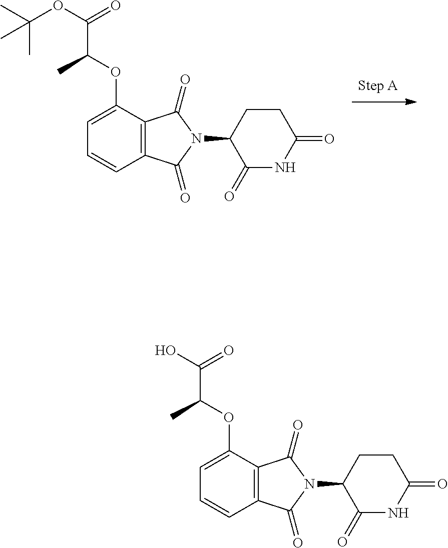 [SMILES: C[C@H](Oc1cccc2c1C(=O)N([C@H]1CCC(=O)NC1=O)C2=O)C(=O)OC(C)(C)C ; C[C@H](Oc1cccc2c1C(=O)N([C@H]1CCC(=O)NC1=O)C2=O)C(=O)O]

To a solution of tert-butyl (S)-2-((2-((S)-2,6-dioxopiperidin-3-yl)-1,3-dioxoisoindolin-4-yl)oxy)propanoate (80 mg, 0.2 mmol) in DCM (3 mL) was added TEA (3 mL) dropwise at 0° C. under nitrogen and the reaction mixture was allowed to stir at room temperature for 16 h. After complete consumption of starting material the volatiles were evaporated under reduced pressure to get the crude compound which was then purified by triturating it by diethyl ether and pentane to afford (S)-2-((2-((S)-2,6-dioxopiperidin-3-yl)-1,3-dioxoisoindolin-4-yl)oxy)propanoic acid (26 mg, 0.075 mmol, 37.5%) of as light brown solid.

LCMS (ESI+): m/z 347.3 [M+H]⁺.

### Step B

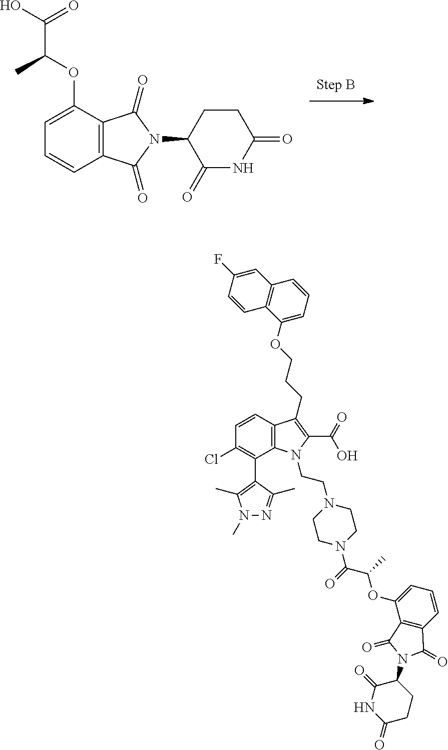 [SMILES: C[C@H](Oc1cccc2c1C(=O)N([C@H]1CCC(=O)NC1=O)C2=O)C(=O)O ; Cc1nn(C)c(C)c1-c1c(Cl)ccc2c(CCCOc3cccc4cc(F)ccc34)c(C(=O)O)n(CCN3CCN(C(=O)[C@H](C)Oc4cccc5c4C(=O)N([C@H]4CCC(=O)NC4=O)C5=O)CC3)c12]

(2S)-2-({2-[(3S)-2,6-Dioxopiperidin-3-yl]-1,3-dioxo-2,3-dihydro-1H-isoindol-4-yl}oxy)propanoic acid (10.1 mg, 0.029 mmol) and HATU (10.3 mg, 0.027 mmol) were dissolved in dry DMF (2.4 mL) under argon atmosphere and DIPEA (0.010 mL, 0.058 mmol) was added. Reaction was stirred in room temperature for 1 h. 6-chloro-3-(3-((6-fluoronaphthalen-1-yl)oxy)propyl)-1-(2-(piperazin-1-yl)ethyl)-7-(1,3,5-trimethyl-1H-pyrazol-4-yl)-1H-indole-2-carboxylic acid hydrochloride (12.7 mg, 0.019 mmol) was dissolved in DMSO (0.600 mL) with DIPEA (0.010 mL, 0.058 mmol) and the solution was slowly added dropwise to the reaction which was then stirred under argon atmosphere in room temperature for 15 min. After complete consumption of the starting material, the solution was diluted with DMSO to 5 mL and crude product was purified with preparative HPLC (H₂O:MeCN+0.1% FA) to give 6-chloro-1-(2-(4-((S)-2-((2-((S)-2,6-dioxopiperidin-3-yl)-1,3-dioxoisoindolin-4-yl)oxy)propanoyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (6.6 mg, 0.007 mmol, 36.0%) as white powder.

LCMS (ESI+): m/z 946 [M+H]⁺

¹H NMR (500 MHz, DMSO, 353 K) w 10.78 (s, 1H), 8.24 (dd, J=9.2, 5.9 Hz, 1H), 7.76 (dd, J=8.5, 7.3 Hz, 1H), 7.69 (d, J=8.5 Hz, 1H), 7.58 (dd, J=10.4, 2.6 Hz, 1H), 7.48-7.37 (m, 3H), 7.32 (td, J=8.9, 2.6 Hz, 1H), 7.22 (dd, J=13.4, 8.5 Hz, 2H), 6.87 (dd, J=5.9, 2.7 Hz, 1H), 5.44 (qd, J=7.0, 6.5, 1.3 Hz, ¹H), 5.04 (dd, J=12.5, 5.5 Hz, 1H), 4.32-4.11 (m, 4H), 3.74 (d, J=4.8 Hz, 3H), 3.42 (bs, 4H), 3.32-3.22 (m, 2H), 2.87 (ddd, J=16.6, 13.4, 5.4 Hz, 1H), 2.65-2.53 (m, 2H), 2.28-2.19 (m, 2H), 2.16-2.02 (m, 7H), 2.02-1.96 (m, 3H), 1.87 (s, 3H), 1.49 (dd, J=6.6, 1.2 Hz, 3H).

### Example 89. 6-Chloro-1-(2-(4-((2S)-2-((2-(2,6-dioxopiperidin-3-yl)-1,3-dioxoisoindolin-4-yl)oxy)propanoyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (318)

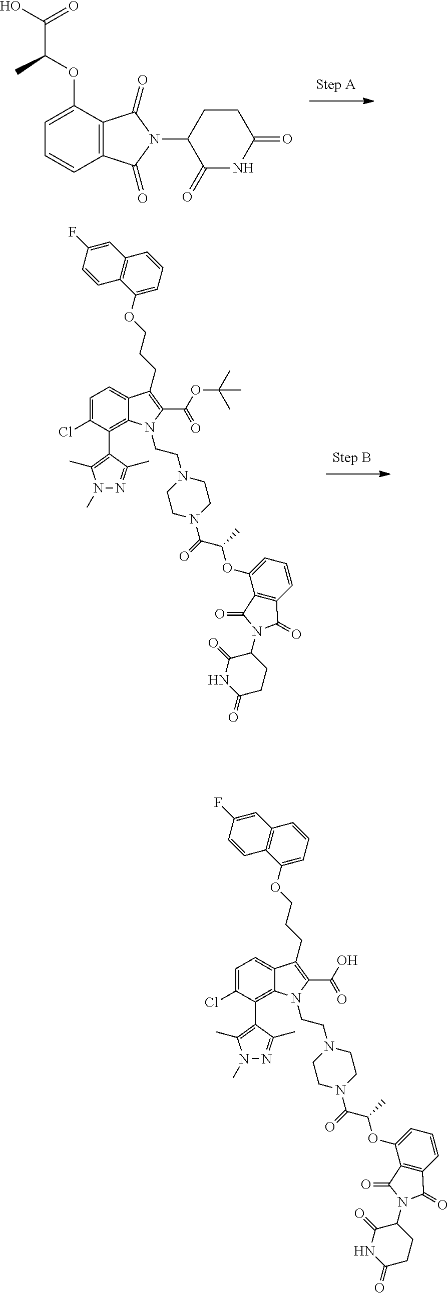 [SMILES: C[C@H](Oc1cccc2c1C(=O)N(C1CCC(=O)NC1=O)C2=O)C(=O)O ; Cc1nn(C)c(C)c1-c1c(Cl)ccc2c(CCCOc3cccc4cc(F)ccc34)c(C(=O)OC(C)(C)C)n(CCN3CCN(C(=O)[C@H](C)Oc4cccc5c4C(=O)N(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)[C@H](C)Oc4cccc5c4C(=O)N(C4CCC(=O)NC4=O)C5=O)CC3)c12]

### Step A

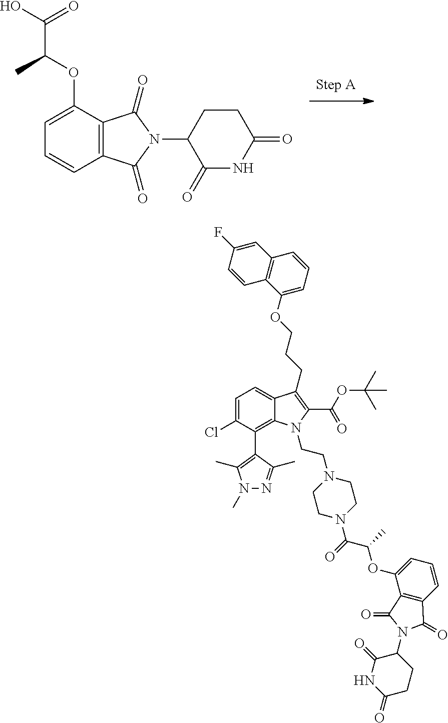 [SMILES: C[C@H](Oc1cccc2c1C(=O)N(C1CCC(=O)NC1=O)C2=O)C(=O)O ; Cc1nn(C)c(C)c1-c1c(Cl)ccc2c(CCCOc3cccc4cc(F)ccc34)c(C(=O)OC(C)(C)C)n(CCN3CCN(C(=O)[C@H](C)Oc4cccc5c4C(=O)N(C4CCC(=O)NC4=O)C5=O)CC3)c12]

An equimolar mixture of (S)-2-((2-((S)-2,6-dioxopiperidin-3-yl)-1,3-dioxoisoindolin-4-yl)oxy)propanoic acid and (S)-2-((2-((R)-2,6-dioxopiperidin-3-yl)-1,3-dioxoisoindolin-4-yl)oxy)propanoic acid (10.0 mg, 0.029 mmol) was dissolved in dry DMF (1.30 mL) and HATU (10.5 mg, 0.028 mmol), followed by DIPEA (0.014 mL, 0.079 mmol) were added. The solution was stirred in room temperature for 15 min and to it, was added tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (17.7 mg, 0.026 mmol). After 75 min of mixing in room temperature LCMS indicated incomplete conversion. Additional portion of HATU (5.25 mg, 0.024 mmol) was added and the reaction was continued for the next 10 min. After complete consumption of the starting material, the reaction was diluted with DCM, washed with brine and water and dried over anhydrous MgSO₄. The solution was filtered and dried under reduced pressure to give crude tert-butyl 6-chloro-1-(2-(4-((2S)-2-((2-(2,6-dioxopiperidin-3-yl)-1,3-dioxoisoindolin-4-yl)oxy)propanoyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (29.2 mg) as green-yellow oil which was used in the next step without further purification.

LCMS (ESI+): m/z 1002.5 [M+H]⁺.

### Step B

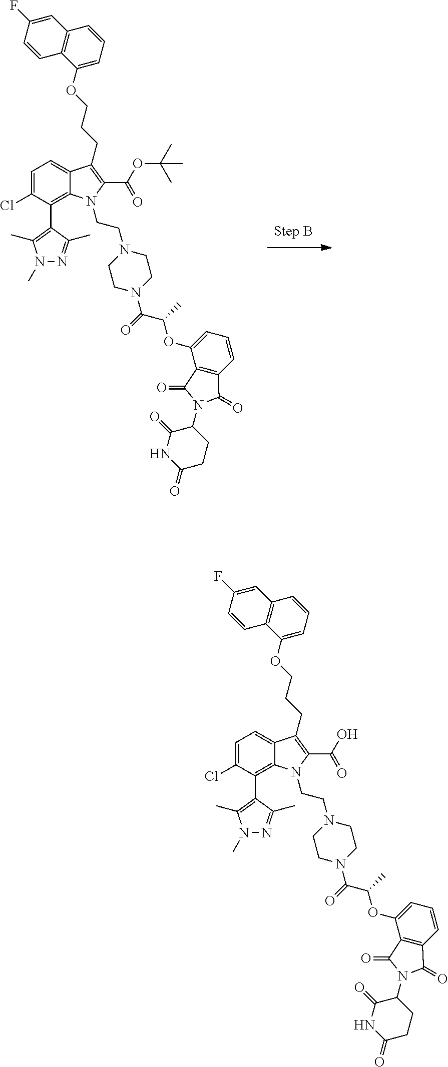 [SMILES: Cc1nn(C)c(C)c1-c1c(Cl)ccc2c(CCCOc3cccc4cc(F)ccc34)c(C(=O)OC(C)(C)C)n(CCN3CCN(C(=O)[C@H](C)Oc4cccc5c4C(=O)N(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)[C@H](C)Oc4cccc5c4C(=O)N(C4CCC(=O)NC4=O)C5=O)CC3)c12]

Crude tert-butyl 6-chloro-1-(2-(4-((2S)-2-((2-(2,6-dioxopiperidin-3-yl)-1,3-dioxoisoindolin-4-yl)oxy)propanoyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (29.2 mg, 0.029 mmol) was dissolved in dry DCM under argon atmosphere and TFA (0.300 mL, 3.918 mmol) was added. Reaction was stirred in room temperature under argon atmosphere for 16 h. After complete consumption of the starting material the solution was concentrated under reduced pressure and the resulting residue was dissolved in DMSO and passed through a syringe filter. Crude product was purified with preparative HPLC (H₂O:MeCN+0.1% FA) to give 6-chloro-1-(2-(4-((2S)-2-((2-(2,6-dioxopiperidin-3-yl)-1,3-dioxoisoindolin-4-yl)oxy)propanoyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (12.4 mg, 0.013 mmol, 50% over 2 steps) as white powder.

LCMS (ESI+): m/z 946 [M+H]⁺

¹H NMR (500 MHz, DMSO, 353 K) δ 10.78 (s, 1H), 8.23 (dd, J=9.2, 5.8 Hz, 1H), 7.76 (dd, J=8.5, 7.3 Hz, 1H), 7.70 (d, J=8.5 Hz, 1H), 7.58 (dd, J=10.4, 2.6 Hz, 1H), 7.48-7.38 (m, 3H), 7.32 (td, J=8.9, 2.6 Hz, 1H), 7.22 (dd, J=9.7, 8.5 Hz, 2H), 6.87 (dd, J=6.0, 2.6 Hz, 1H), 5.50-5.39 (m, 1H), 5.04 (dd, J=12.5, 5.5 Hz, 1H), 4.32-4.21 (m, 3H), 4.21-4.13 (m, 1H), 3.74 (d, J=4.1 Hz, 3H), 3.42 (s, 4H), 3.32-3.23 (m, 2H), 2.87 (ddd, J=17.3, 13.5, 5.3 Hz, 1H), 2.63-2.51 (m, 2H), 2.28-2.19 (m, 2H), 2.16-2.02 (m, 7H), 2.02-1.97 (m, 3H), 1.87 (s, 3H), 1.49 (d, J=6.5 Hz, 3H).

### Example 90. 6-Chloro-1-(2-(4-(2-((2-(2,6-dioxopiperidin-3-yl)-1,3-dioxoisoindolin-4-yl)oxy)ethyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (321)

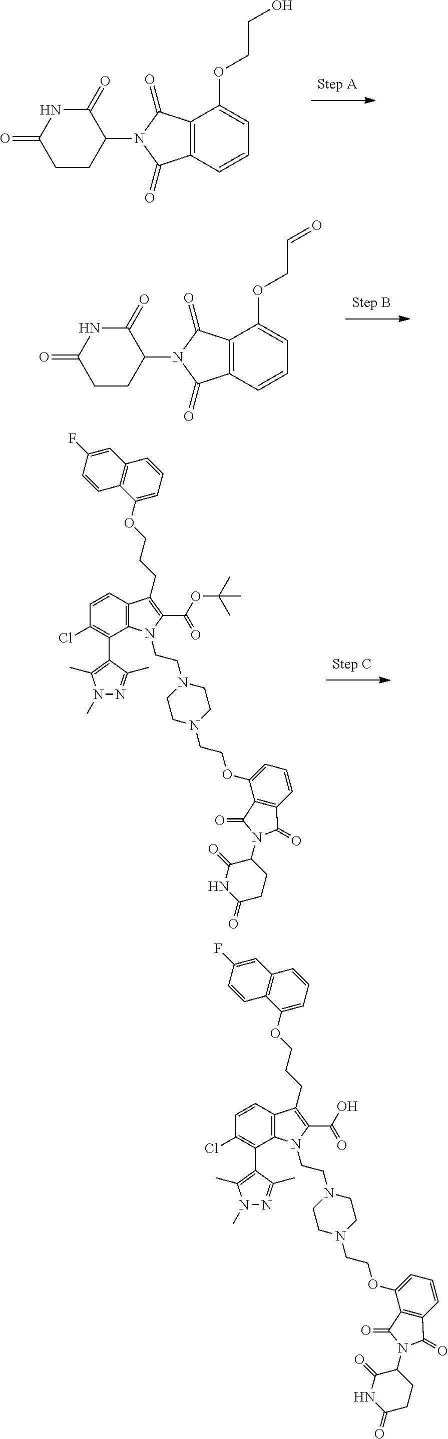

### Step C

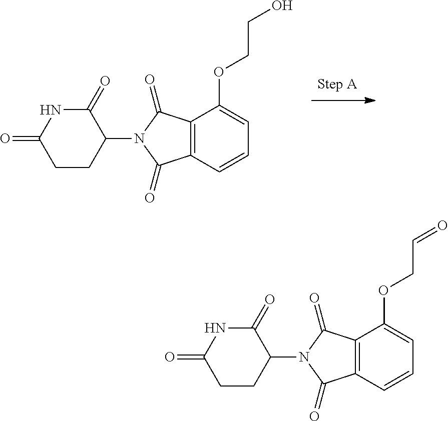 [SMILES: O=C1CCC(N2C(=O)c3cccc(OCCO)c3C2=O)C(=O)N1 ; O=CCOc1cccc2c1C(=O)N(C1CCC(=O)NC1=O)C2=O]

To a solution of 2-(2,6-dioxopiperidin-3-yl)-4-(2-hydroxyethoxy)-2,3-dihydro-1H-isoindole-1,3-dione (116.0 mg, 0.364 mmol) in dry DCM (3.6 mL) was added DMSO (0.518 mL, 7.289 mmol) and DIPEA (0.317 mL, 1.822 mmol). The solution was cooled to 0° C. and SO₃-pyridine complex (232.0 mg, 1.458 mmol) was added. The reaction mixture was stirred at 0° C. for next 1 h. The resulting solution was warmed to room temperature over 30 min. The crude was extracted with 3N HCl and brine. The organic layer was dried over Na₂SO₄, filtered and concentrated in vacuo to give a 2-{[2-(2,6-dioxopiperidin-3-yl)-1,3-dioxo-2,3-dihydro-1H-isoindol-4-yl]oxy}acetaldehyde (65.0 mg, crude).

### Step B

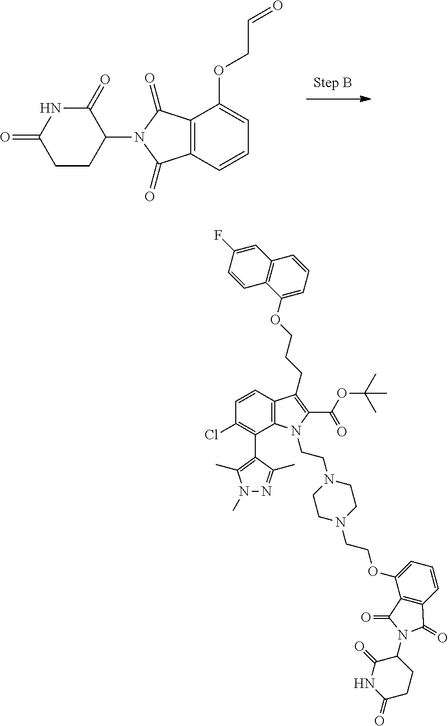 [SMILES: O=CCOc1cccc2c1C(=O)N(C1CCC(=O)NC1=O)C2=O ; Cc1nn(C)c(C)c1-c1c(Cl)ccc2c(CCCOc3cccc4cc(F)ccc34)c(C(=O)OC(C)(C)C)n(CCN3CCN(CCOc4cccc5c4C(=O)N(C4CCC(=O)NC4=O)C5=O)CC3)c12]

tert-Butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (60.0 mg, 0.089 mmol) and 2-{[2-(2,6-dioxopiperidin-3-yl)-1,3-dioxo-2,3-dihydro-1H-isoindol-4-yl]oxy}acetaldehyde (56.3 mg, 0.178 mmol) were dissolved in dry DCM (0.890 mL) and the mixture was stirred for 30 min. Subsequently, the mixture was cooled to 0° C. and NaBH(OAc)₃ (226.3 mg, 1.068 mmol) was added and the mixture was stirred at room temperature for 96 h, after which time full conversion was observed. Reaction mixture was quenched with water, extracted with DCM, washed with saturated NaHCO₃ solution and brine, dried over anhydrous Na₂SO₄ and concentrated in vacuo. Then, crude mixture was purified by preparative TLC (twice 5% MeOH in DCM) to give a tert-butyl 6-chloro-1-{2-[4-(2-{[2-(2,6-dioxopiperidin-3-yl)-1,3-dioxo-2,3-dihydro-1H-isoindol-4-yl]oxy}ethyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (26.0 mg, 0.027 mmol, 30.0%) as a white solid.

LCMS (ESI+): m/z 974.3 [M+H]⁺

### Step C

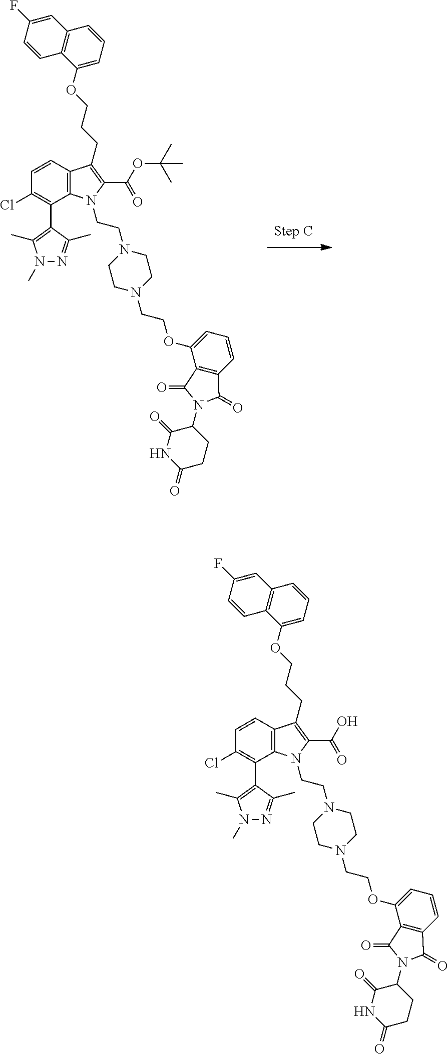 [SMILES: Cc1nn(C)c(C)c1-c1c(Cl)ccc2c(CCCOc3cccc4cc(F)ccc34)c(C(=O)OC(C)(C)C)n(CCN3CCN(CCOc4cccc5c4C(=O)N(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(CCOc4cccc5c4C(=O)N(C4CCC(=O)NC4=O)C5=O)CC3)c12]

To a solution of tert-butyl 6-chloro-1-{2-[4-(2-{[2-(2,6-dioxopiperidin-3-yl)-1,3-dioxo-2,3-dihydro-1H-isoindol-4-yl]oxy}ethyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (26.0 mg, 0.027 mmol) in DCM (0.500 mL) was added TFA (0.300 mL, 3.918 mmol). The mixture was stirred for 36 h at RT. The crude was concentrated in vacuo and purified by preparative HPLC (C18, H₂O:MeCN+0.1% FA) to give 6-chloro-1-{2-[4-(2-{[2-(2,6-dioxopiperidin-3-yl)-1,3-dioxo-2,3-dihydro-1H-isoindol-4-yl]oxy}ethyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (11.5 mg, 0.013 mmol, 46.9%) as a white solid.

LCMS (ESI+): m/z 918.3 [M+H]⁺

¹H NMR (500 MHz, DMSO) δ 10.78 (s, 1H), 8.24 (dd, J=9.2, 5.9 Hz, 1H), 7.78 (dd, J=8.5, 7.2 Hz, 1H), 7.69 (d, J=8.5 Hz, 1H), 7.58 (dd, J=10.4, 2.6 Hz, 1H), 7.47 (d, J=8.5 Hz, 1H), 7.45-7.40 (m, 3H), 7.32 (td, J=8.9, 2.6 Hz, 1H), 7.21 (d, J=8.6 Hz, 1H), 6.86 (dd, J=5.3, 3.3 Hz, 1H), 5.03 (dd, J=12.4, 5.5 Hz, 1H), 4.31-4.20 (m, 5H), 4.19-4.10 (m, 1H), 3.74 (s, 3H), 3.30-3.23 (m, 2H), 2.93-2.82 (m, 1H), 2.74-2.68 (m, 2H), 2.66-2.54 (m, 3H), 2.28-2.19 (m, 2H), 2.21-2.15 (m, 4H), 2.17-2.08 (m, 2H), 2.10-2.02 (m, 1H), 2.01 (s, 3H), 1.88 (s, 3H).

3 protons in aliphatic area overlap with DMSO.

### Example 91. 6-Chloro-1-(2-(4-(2-((2-(2,6-dioxopiperidin-3-yl)-1-oxoisoindolin-4-yl)thio)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (322)

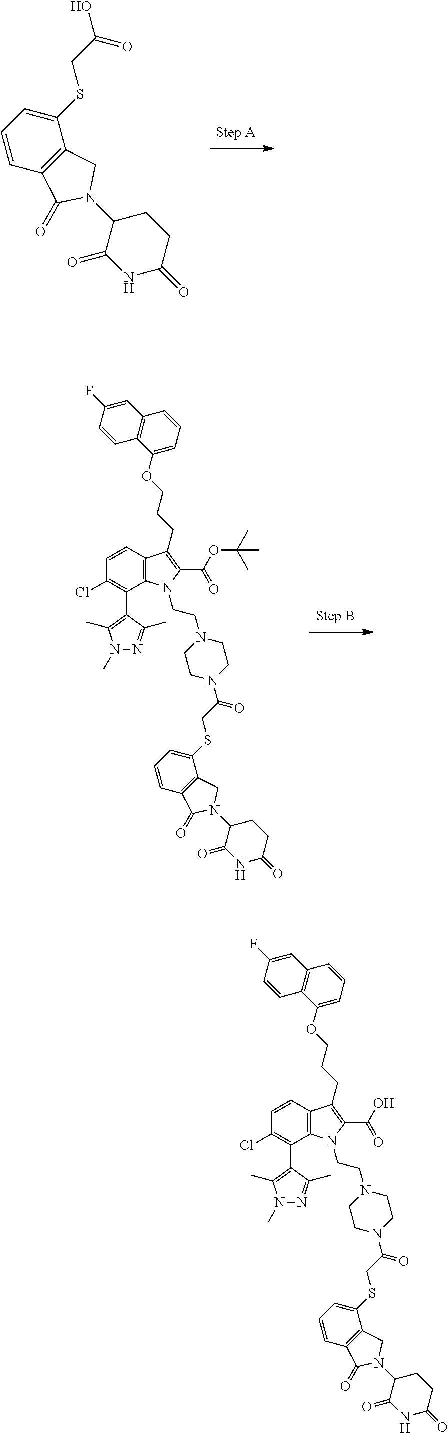 [SMILES: O=C(O)CSc1cccc2c1CN(C1CCC(=O)NC1=O)C2=O ; Cc1nn(C)c(C)c1-c1c(Cl)ccc2c(CCCOc3cccc4cc(F)ccc34)c(C(=O)OC(C)(C)C)n(CCN3CCN(C(=O)CSc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)CSc4cccc5c4CN(C4CCC(=O)NC4=O)C5=O)CC3)c12]

### Step A

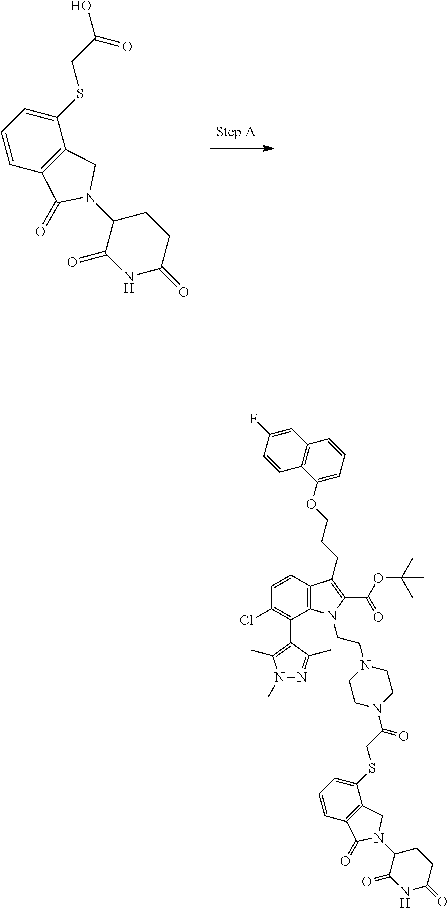 [SMILES: O=C(O)CSc1cccc2c1CN(C1CCC(=O)NC1=O)C2=O ; Cc1nn(C)c(C)c1-c1c(Cl)ccc2c(CCCOc3cccc4cc(F)ccc34)c(C(=O)OC(C)(C)C)n(CCN3CCN(C(=O)CSc4cccc5c4CN(C4CCC(=O)NC4=O)C5=O)CC3)c12]

2-{[2-(2,6-Dioxopiperidin-3-yl)-1-oxo-2,3-dihydro-1H-isoindol-4-yl]sulfanyl}acetic acid (13.6 mg, 0.041 mmol) was dissolved under argon atmosphere in dry DMF (0.742 mL) and DIPEA (0.019 mL, 0.111 mmol) along with HATU (14.8 mg, 0.039 mmol) were added. The solution was stirred at room temperature for 15 min under argon. To it, tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (25.0 mg, 0.037 mmol) was added and the reaction was continued at room temperature. After 90 min additional portion of 2-{[2-(2,6-dioxopiperidin-3-yl)-1-oxo-2,3-dihydro-1H-isoindol-4-yl]sulfanyl}acetic acid (6.8 mg, 0.020 mmol), HATU (7.4 mg, 0.019 mmol) and DIPEA (0.009 mL, 0.055 mmol) were added and the reaction was heated to 60° C. for next 30 min. The solution was diluted with DCM and washed with brine and water. Organic layer was collected, dried over anhydrous MgSO₄, filtered and dried under reduced pressure to give 59.9 mg crude tert-butyl 6-chloro-1-(2-(4-(2-((2-(2,6-dioxopiperidin-3-yl)-1-oxoisoindolin-4-yl)thio)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate, that was used in the next step without further purification.

LCMS (ESI+): m/z 990.15 [M+H]⁺

### Step B

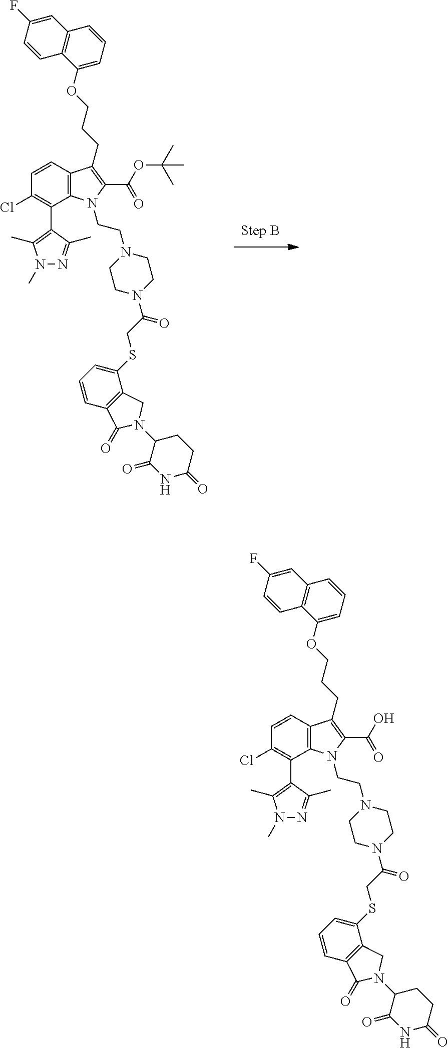 [SMILES: Cc1nn(C)c(C)c1-c1c(Cl)ccc2c(CCCOc3cccc4cc(F)ccc34)c(C(=O)OC(C)(C)C)n(CCN3CCN(C(=O)CSc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)CSc4cccc5c4CN(C4CCC(=O)NC4=O)C5=O)CC3)c12]

tert-Butyl 6-chloro-1-(2-(4-(2-((2-(2,6-dioxopiperidin-3-yl)-1-oxoisoindolin-4-yl)thio)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (59.9 mg, crude) was dissolved in dry DCM (0.600 mL) under argon atmosphere and TFA (0.600 mL) was added. The reaction mixture was stirred at room temperature for 16 h. After complete consumption of the starting material, the solution was concentrated under reduced pressure and purified with preparative HPLC (C18, H₂O:MeCN+0.1% FA) and preparative TLC (SiO₂, 40% MeOH in DCM) to give 6-chloro-1-(2-(4-(2-((2-(2,6-dioxopiperidin-3-yl)-1-oxoisoindolin-4-yl)thio)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (10.8 mg, 0.012 mmol, 32% over 2 steps) as white solid.

LCMS (ESI+): m/z 934.1 [M+H]⁺

¹H NMR (500 MHz, DMSO, 353 K) δ 10.66 (s, 1H), 8.24 (dd, J=9.2, 5.9 Hz, 1H), 7.75-7.66 (m, 2H), 7.63-7.55 (m, 2H), 7.50 (t, J=7.6 Hz, 1H), 7.45-7.38 (m, 2H), 7.32 (ddd, J=9.3, 8.6, 2.7 Hz, 1H), 7.21 (d, J=8.5 Hz, 1H), 6.87 (dd, J=5.8, 2.8 Hz, 1H), 5.07 (dd, J=13.0, 5.2 Hz, 1H), 4.42 (d, J=17.2 Hz, 1H), 4.33 (d, J=17.2 Hz, 1H), 4.30-4.22 (m, 3H), 4.17 (ddd, J=14.7, 8.2, 6.4 Hz, 1H), 3.96 (s, 2H), 3.76 (d, J=1.2 Hz, 3H), 3.40-3.32 (m, 4H), 3.32-3.24 (m, 2H), 2.88 (ddd, J=17.3, 13.4, 5.5 Hz, 1H), 2.63 (ddd, J=17.3, 4.6, 2.5 Hz, 1H), 2.44 (td, J=13.1, 4.6 Hz, 1H), 2.28-2.20 (m, 2H), 2.16-2.02 (m, 7H), 2.01 (s, 3H), 1.88 (s, 3H).

### Example 92. 6-Chloro-1-(2-(4-(3-(2-(2,6-dioxopiperidin-3-yl)-1-oxoisoindolin-4-yl)propanoyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (323)

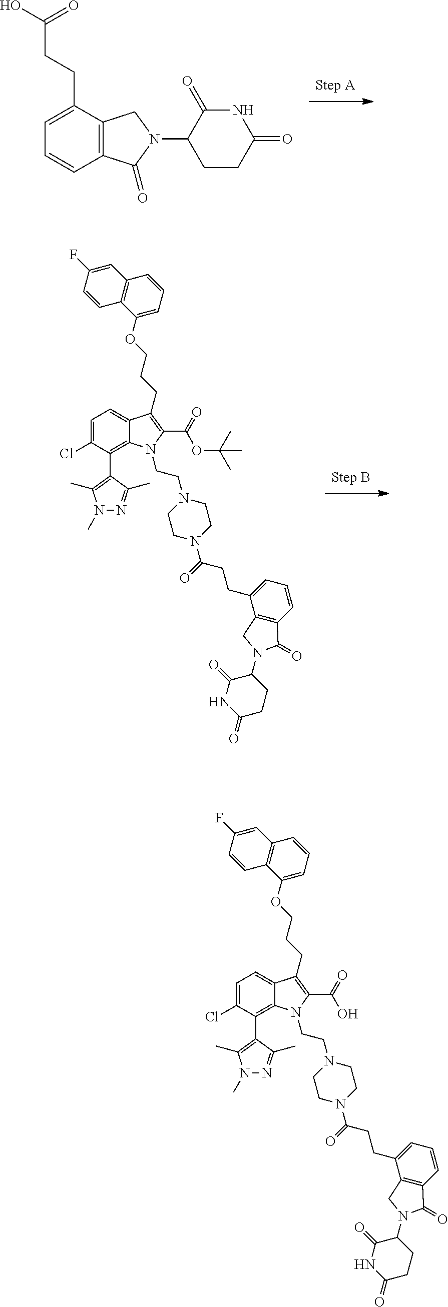 [SMILES: O=C(O)CCc1cccc2c1CN(C1CCC(=O)NC1=O)C2=O ; Cc1nn(C)c(C)c1-c1c(Cl)ccc2c(CCCOc3cccc4cc(F)ccc34)c(C(=O)OC(C)(C)C)n(CCN3CCN(C(=O)CCc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)CCc4cccc5c4CN(C4CCC(=O)NC4=O)C5=O)CC3)c12]

### Step A

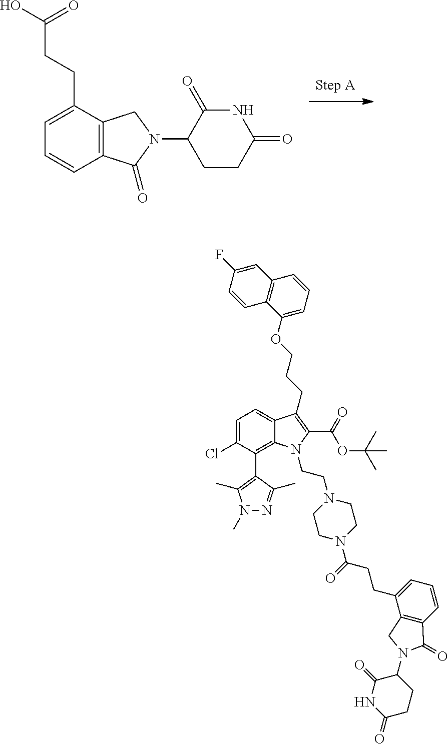 [SMILES: O=C(O)CCc1cccc2c1CN(C1CCC(=O)NC1=O)C2=O ; Cc1nn(C)c(C)c1-c1c(Cl)ccc2c(CCCOc3cccc4cc(F)ccc34)c(C(=O)OC(C)(C)C)n(CCN3CCN(C(=O)CCc4cccc5c4CN(C4CCC(=O)NC4=O)C5=O)CC3)c12]

tert-Butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (25.0 mg, 0.037 mmol) and 3-[2-(2,6-dioxopiperidin-3-yl)-1-oxo-2,3-dihydro-1H-isoindol-4-yl]propanoic acid (12.9 mg, 0.041 mmol) were dissolved in dry DMF (0.742 mL) under argon atmosphere. Then DIPEA (0.019 mL, 0.111 mmol) followed by HATU (14.8 mg, 0.039 mmol) were added and the reaction was stirred under argon at room temperature for 30 min. After complete consumption of the starting material, the solution was diluted with DCM and washed with brine and water. Organic layer was dried over anhydrous MgSO₄, filtered and dried under reduced pressure to give 39.8 mg crude of tert-butyl 6-chloro-1-[2-(4-{3-[2-(2,6-dioxopiperidin-3-yl)-1-oxo-2,3-dihydro-1H-isoindol-4-yl]propanoyl}piperazin-1-yl)ethyl]-3-{3-[(6-fluoronaphthalen-1-yl)oxy]propyl}-7-(1,3,5-trimethyl-1H-pyrazol-4-yl)-1H-indole-2-carboxylate as yellow oil, which was used in the next step without further purification.

LCMS (ESI+): m/z 973.0 [M+H]⁺

### Step B

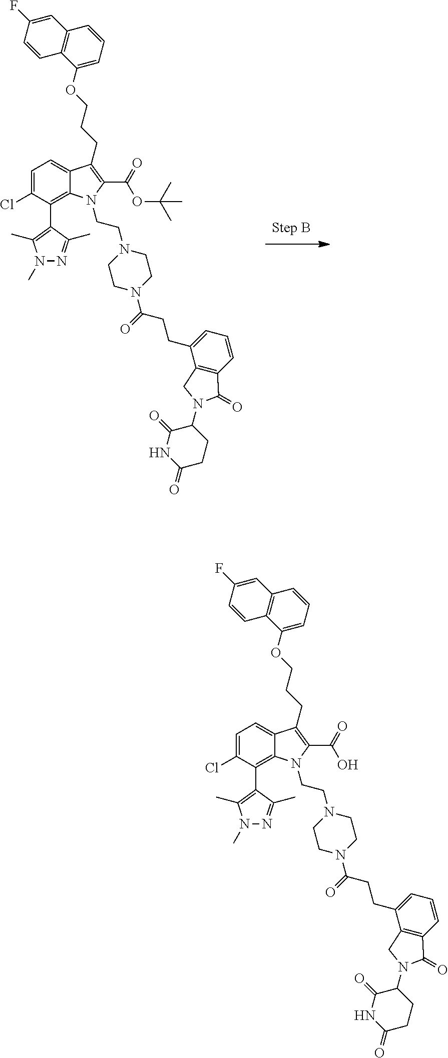 [SMILES: Cc1nn(C)c(C)c1-c1c(Cl)ccc2c(CCCOc3cccc4cc(F)ccc34)c(C(=O)OC(C)(C)C)n(CCN3CCN(C(=O)CCc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)CCc4cccc5c4CN(C4CCC(=O)NC4=O)C5=O)CC3)c12]

Crude tert-butyl 6-chloro-1-[2-(4-{3-[2-(2,6-dioxopiperidin-3-yl)-1-oxo-2,3-dihydro-1H-isoindol-4-yl]propanoyl}piperazin-1-yl)ethyl]-3-{3-[(6-fluoronaphthalen-1-yl)oxy]propyl}-7-(1,3,5-trimethyl-1H-pyrazol-4-yl)-1H-indole-2-carboxylate (39.8 mg) was dissolved in dry DCM (0.400 mL) under argon atmosphere and TFA (0.400 mL, 5.224 mmol) was added. Reaction was stirred under argon for 16 h at room temperature. After complete consumption of the starting material, the solution was concentrated and the resulting residue dissolved in DMSO. Crude product was purified with preparative HPLC (C18, H₂O:MeCN+0.1% FA) to give 6-chloro-1-(2-(4-(3-(2-(2,6-dioxopiperidin-3-yl)-1-oxoisoindolin-4-yl)propanoyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (17.2 mg, 0.019 mmol, 51% over 2 steps) as white solid.

LCMS (ESI+): m/z 916.15 [M+H]⁺

¹H NMR (500 MHz, DMSO, 353 K) δ 10.64 (s, 1H), 8.24 (dd, J=9.2, 5.9 Hz, 1H), 7.70 (d, J=8.5 Hz, 1H), 7.58 (dd, J=10.4, 2.6 Hz, 1H), 7.55 (dd, J=7.1, 1.5 Hz, 1H), 7.48-7.37 (m, 4H), 7.35-7.29 (m, 1H), 7.24-7.19 (m, 1H), 6.87 (dd, J=6.0, 2.6 Hz, 1H), 5.06 (dd, J=13.0, 5.3 Hz, 1H), 4.48 (dd, J=16.9, 1.6 Hz, 1H), 4.38 (dd, J=16.9, 1.9 Hz, 1H), 4.30-4.20 (m, 3H), 4.20-4.12 (m, 1H), 3.75 (s, 3H), 3.33-3.25 (m, 6H), 2.93-2.85 (m, 3H), 2.68-2.60 (m, 3H), 2.44 (qd, J=13.1, 4.7 Hz, 1H), 2.28-2.19 (m, 2H), 2.13-2.01 (m, 7H), 2.00 (s, 3H), 1.88 (s, 3H).

### Example 93. 6-Chloro-1-{2-[4-(3-{[2-(2,6-dioxopiperidin-3-yl)-1-oxo-2,3-dihydro-1H-isoindol-4-yl]oxy}propanoyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (324)

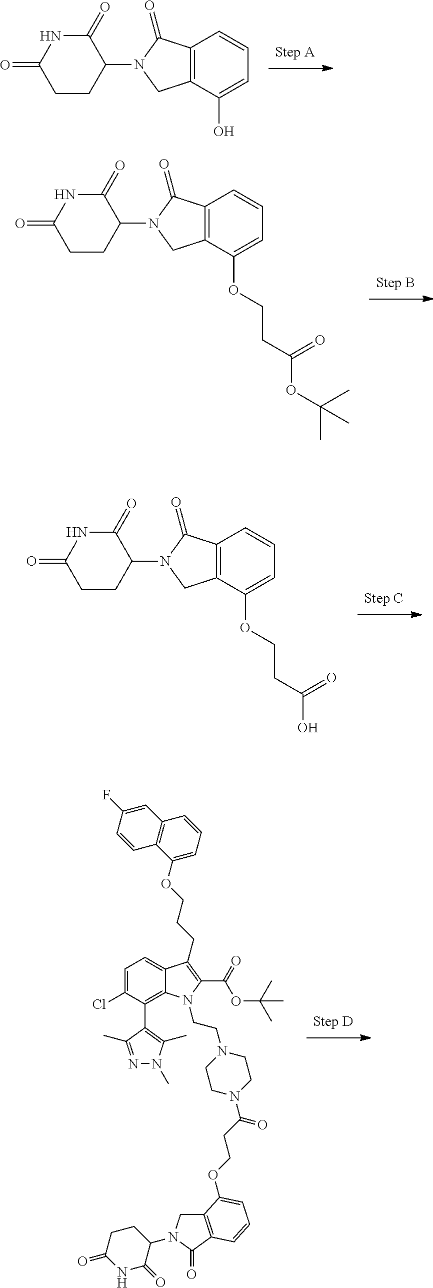 [SMILES: O=C1CCC(N2Cc3c(O)cccc3C2=O)C(=O)N1 ; CC(C)(C)OC(=O)CCOc1cccc2c1CN(C1CCC(=O)NC1=O)C2=O ; O=C(O)CCOc1cccc2c1CN(C1CCC(=O)NC1=O)C2=O ; Cc1nn(C)c(C)c1-c1c(Cl)ccc2c(CCCOc3cccc4cc(F)ccc34)c(C(=O)OC(C)(C)C)n(CCN3CCN(C(=O)CCOc4cccc5c4CN(C4CCC(=O)NC4=O)C5=O)CC3)c12]

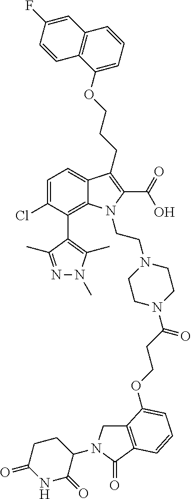 [SMILES: Cc1nn(C)c(C)c1-c1c(Cl)ccc2c(CCCOc3cccc4cc(F)ccc34)c(C(=O)O)n(CCN3CCN(C(=O)CCOc4cccc5c4CN(C4CCC(=O)NC4=O)C5=O)CC3)c12]

### Step A

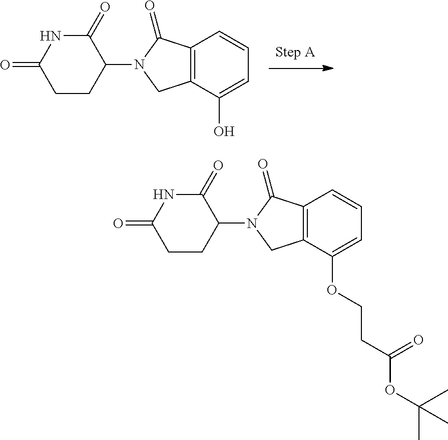

To a stirred solution of 3-(4-hydroxy-1-oxo-2,3-dihydro-1H-isoindol-2-yl)piperidine-2,6-dione (100.0 mg, 0.384 mmol) and tert-butyl 3-bromopropionate (160.7 mg, 0.768 mmol) in dry DMF (2.0 mL) were added K₂CO₃ (159.3 mg, 1.153 mmol) and KI (63.8 mg, 0.384 mmol). The reaction mixture was stirred at 60° C. for 2 days. The crude was diluted in DCM and washed with water and brine. The organic layer was dried over MgSO₄, filtered and concentrated in vacuo to obtain tert-butyl 3-{[2-(2,6-dioxopiperidin-3-yl)-1-oxo-2,3-dihydro-1H-isoindol-4-yl]oxy}propanoate (98 mg, crude) as an yellow oil which was used in the next step without further purification.

### Step B

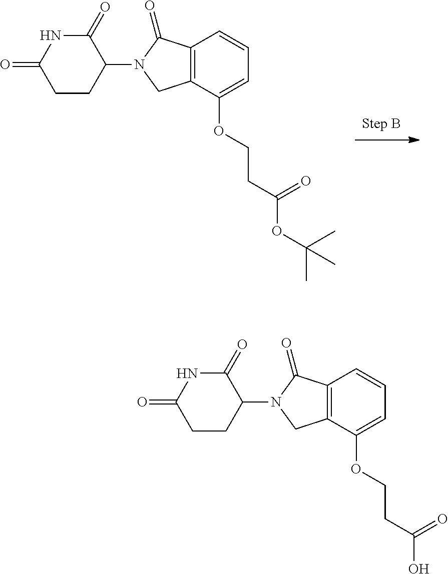 [SMILES: CC(C)(C)OC(=O)CCOc1cccc2c1CN(C1CCC(=O)NC1=O)C2=O ; O=C(O)CCOc1cccc2c1CN(C1CCC(=O)NC1=O)C2=O]

To a solution of tert-butyl 3-{[2-(2,6-dioxopiperidin-3-yl)-1-oxo-2,3-dihydro-1H-isoindol-4-yl]oxy}propanoate (98.0 mg, crude) in DCM (2.0 mL) was added TFA (1.0 mL, 13.059 mmol) and the mixture was stirred for next 18 h at RT. The crude was concentrated in vacuo and purified by reverse phase flash chromatography (C18, H₂O:MeCN+0.1% FA) to obtain 3-{[2-(2,6-dioxopiperidin-3-yl)-1-oxo-2,3-dihydro-1H-isoindol-4-yl]oxy}propanoic acid (68.0 mg, 0.205 mmol, 53.4% yield over two steps) as a white solid.

LCMS (ESI+): m/z 333.1 [M+H]⁺

### Step C

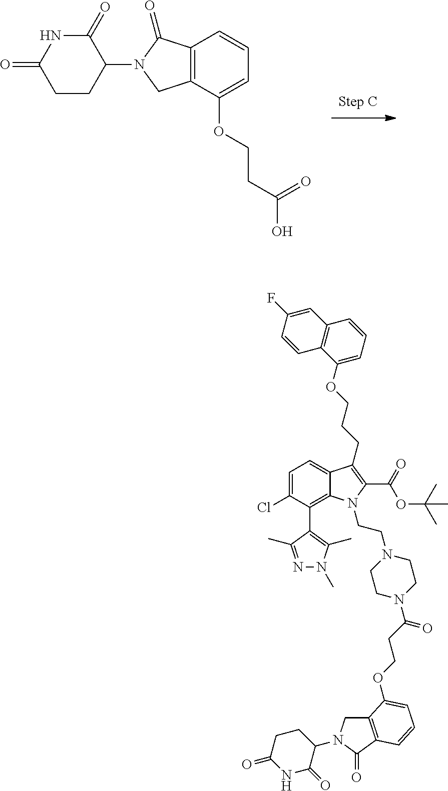 [SMILES: O=C(O)CCOc1cccc2c1CN(C1CCC(=O)NC1=O)C2=O ; Cc1nn(C)c(C)c1-c1c(Cl)ccc2c(CCCOc3cccc4cc(F)ccc34)c(C(=O)OC(C)(C)C)n(CCN3CCN(C(=O)CCOc4cccc5c4CN(C4CCC(=O)NC4=O)C5=O)CC3)c12]

To a solution of tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (17.0 mg, 0.025 mmol) and 3-{[2-(2,6-dioxopiperidin-3-yl)-1-oxo-2,3-dihydro-1-isoindol-4-yl]oxy}propanoic acid (12.6 mg, 0.038 mmol) in dry DMF (2.0 mL) were added HATU (19.2 mg, 0.050 mmol) and DIPEA (0.022 mL, 0.126 mmol). The reaction was stirred at room temperature for 30 min. The crude was diluted in DCM and washed with brine. The organic layer was dried over MgSO₄, filtered and concentrated in vacuo. The residues were dissolved in DMSO and purified by preparative HPLC (H₂O:MeCN+0.1% FA) to obtain tert-butyl 6-chloro-1-{2-[4-(3-{[2-(2,6-dioxopiperidin-3-yl)-1-oxo-2,3-dihydro-1H-isoindol-4-yl]oxy}propanoyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (15.0 mg, 0.015 mmol, 60.2%) as a white solid.

LCMS (ESI+): m/z 988.25 [M+H]⁺

### Step D

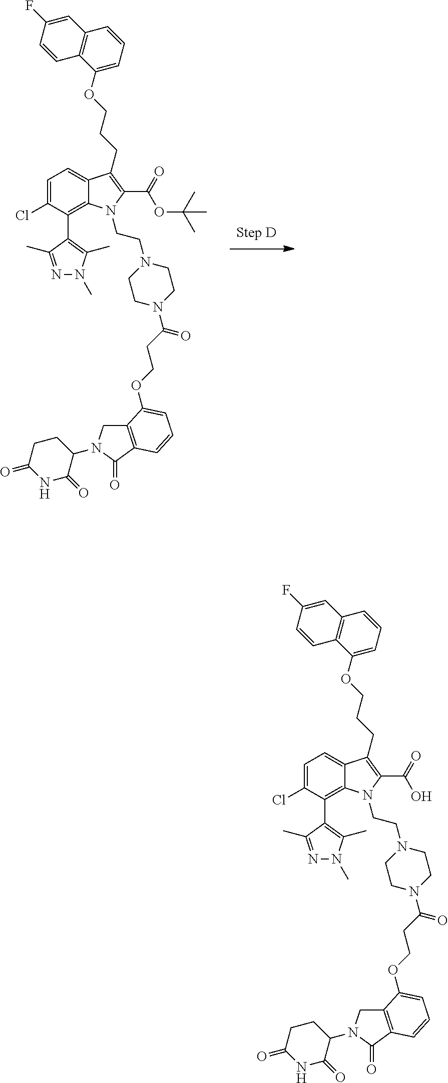 [SMILES: Cc1nn(C)c(C)c1-c1c(Cl)ccc2c(CCCOc3cccc4cc(F)ccc34)c(C(=O)OC(C)(C)C)n(CCN3CCN(C(=O)CCOc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)CCOc4cccc5c4CN(C4CCC(=O)NC4=O)C5=O)CC3)c12]

To a solution of tert-butyl 6-chloro-1-{2-[4-(3-{[2-(2,6-dioxopiperidin-3-yl)-1-oxo-2,3-dihydro-1H-isoindol-4-yl]oxy}propanoyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (15.0 mg, 0.015 mmol) in DCM (1.0 mL) was added TFA (1.0 mL, 13.059 mmol) and the mixture was stirred for next 18 h at RT. The crude was concentrated in vacuo and purified by reverse phase flash chromatography (C18, H₂O:MeCN+0.1% FA) to obtain 6-chloro-1-{2-[4-(3-{[2-(2,6-dioxopiperidin-3-yl)-1-oxo-2,3-dihydro-1H-isoindol-4-yl]oxy}propanoyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (6.8 mg, 0.007 mmol, 48.1%) as a white solid.

LCMS (ESI+): m/z 932.2 [M+H]⁺

¹H NMR (500 MHz, DMSO, 353 K) δ 8.24 (dd, J=9.2, 5.9 Hz, 1H), 7.68 (d, J=8.5 Hz, 1H), 7.58 (dd, J=10.4, 2.6 Hz, 1H), 7.43-7.39 (m, 2H), 7.34-7.30 (m, 2H), 7.21-7.17 (m, 2H), 7.03 (dt, J=8.0, 0.8 Hz, 1H), 6.86 (dd, J=6.3, 2.4 Hz, 1H), 5.10 (ddd, J=13.1, 5.2, 1.2 Hz, 1H), 4.35 (d, J=16.9 Hz, 1H), 4.32-4.23 (m, 4H), 4.16 (ddd, J=13.9, 8.1, 5.8 Hz, 1H), 3.92-3.80 (m, 2H), 3.74 (d, J=1.8 Hz, 3H), 3.31 (t, J=4.9 Hz, 4H), 3.28-3.24 (m, 2H), 2.77 (dt, J=17.3, 3.9 Hz, 1H), 2.48-2.33 (m, 3H), 2.26-2.20 (m, 2H), 2.15-2.03 (m, 7H), 1.99 (d, J=3.1 Hz, 3H), 1.87 (s, 3H).

1 proton in aliphatic area overlaps with water.

### Example 94. 6-Chloro-1-[4-(2-{[2-(2,6-dioxopiperidin-3-yl)-1,3-dioxo-2,3-dihydro-1H-isoindol-4-yl]amino}acetamido)butyl]-3-{3-[(6-fluoronaphthalen-1-yl)oxy]propyl}-7-(1,3,5-trimethyl-1H-pyrazol-4-yl)-1H-indole-2-carboxylic acid (325)

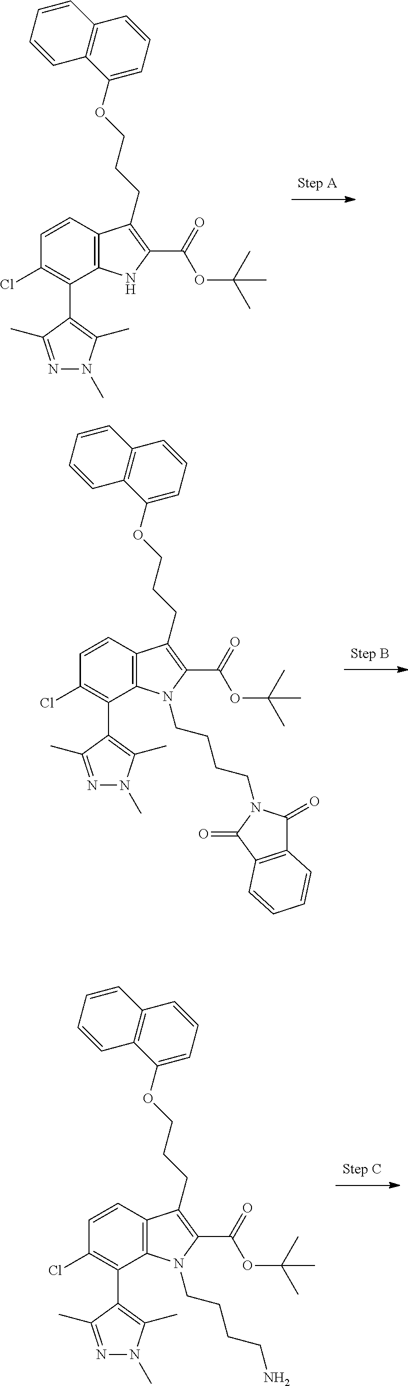

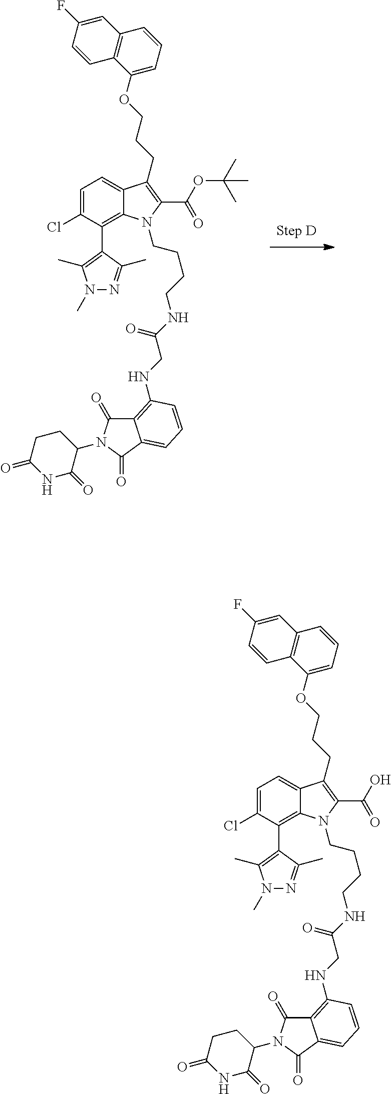 [SMILES: Cc1nn(C)c(C)c1-c1c(Cl)ccc2c(CCCOc3cccc4cc(F)ccc34)c(C(=O)OC(C)(C)C)n(CCCCNC(=O)CNc3cccc4c3C(=O)N(C3CCC(=O)NC3=O)C4=O)c12 ; Cc1nn(C)c(C)c1-c1c(Cl)ccc2c(CCCOc3cccc4cc(F)ccc34)c(C(=O)O)n(CCCCNC(=O)CNc3cccc4c3C(=O)N(C3CCC(=O)NC3=O)C4=O)c12]

### Step A

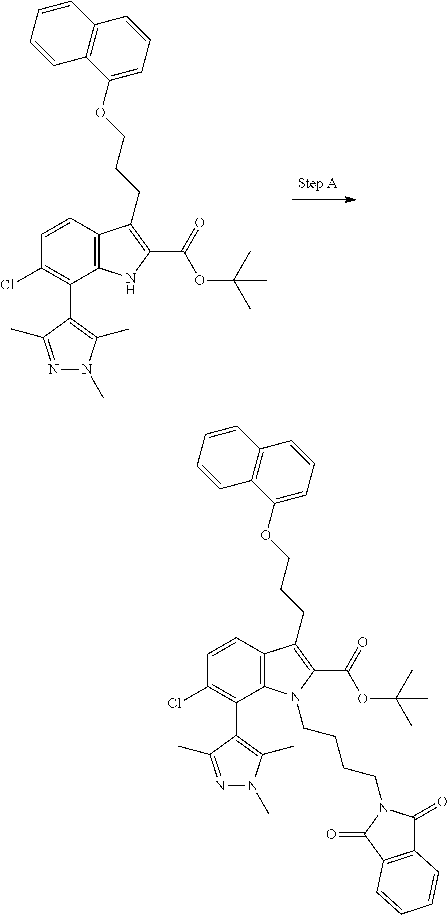 [SMILES: Cc1nn(C)c(C)c1-c1c(Cl)ccc2c(CCCOc3cccc4ccccc34)c(C(=O)OC(C)(C)C)[nH]c12 ; Cc1nn(C)c(C)c1-c1c(Cl)ccc2c(CCCOc3cccc4ccccc34)c(C(=O)OC(C)(C)C)n(CCCCN3C(=O)c4ccccc4C3=O)c12]

To a well stirred solution of tert-butyl 6-chloro-3-(3-(naphthalen-1-yloxy)propyl)-7-(1,3,5-trimethyl-1H-pyrazol-4-yl)-1H-indole-2-carboxylate (200 mg, 0.357 mmol) in DMF (5 mL) was added 2-(4-bromobutyl)isoindoline-1,3-dione (200 mg, 0.713 mmol) followed by Cs₂CO₃ (347 mg, 1.07 mmol) in DMF and the mixture was allowed to stir at 90° C. for 16 h under nitrogen. After complete consumption of the starting material the reaction mixture was diluted with EtOAc, washed successively with water and brine, the organic layer was dried over Na₂SO₄ and evaporated under reduced pressure to get 250 mg the crude compound tert-butyl 6-chloro-1-(4-(1,3-dioxoisoindolin-2-yl)butyl)-3-(3-(naphthalen-1-yloxy)propyl)-7-(1,3,5-trimethyl-1H-pyrazol-4-yl)-1H-indole-2-carboxylate, which was used for the next step without further purification.

LCMS (ESI+): m/z 781.4 [M+H]⁺

### Step B

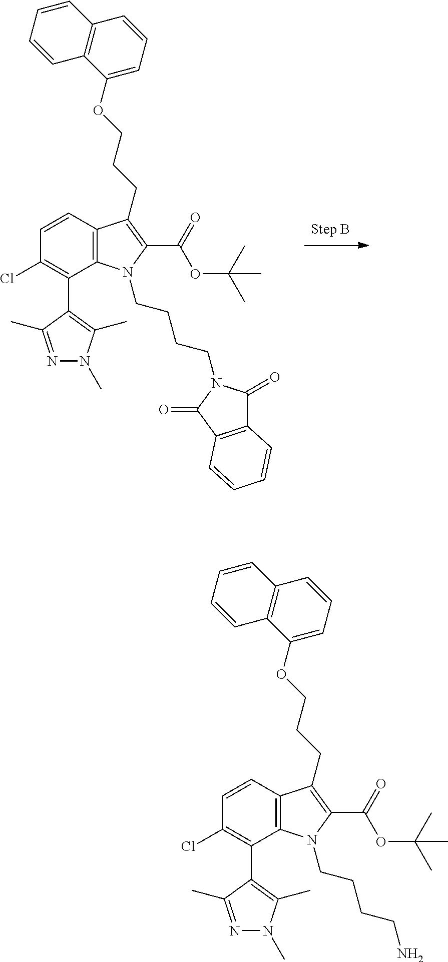 [SMILES: Cc1nn(C)c(C)c1-c1c(Cl)ccc2c(CCCOc3cccc4ccccc34)c(C(=O)OC(C)(C)C)n(CCCCN3C(=O)c4ccccc4C3=O)c12 ; Cc1nn(C)c(C)c1-c1c(Cl)ccc2c(CCCOc3cccc4ccccc34)c(C(=O)OC(C)(C)C)n(CCCCN)c12]

A mixture of tert-butyl 6-chloro-1-(4-(1,3-dioxoisoindolin-2-yl)butyl)-3-(3-(naphthalen-1-yloxy)propyl)-7-(1,3,5-trimethyl-1H-pyrazol-4-yl)-1H-indole-2-carboxylate (250 mg, crude) and hydrazine hydrate (75 mg, 1.64 mmol) in dry tert-butanol (3 mL) was refluxed for 2 h at 140° C. under nitrogen. Then it was allowed to cool down to room temperature. The volatiles were evaporated under reduced pressure. The residue was purified by preparative HPLC (C18, 20 mM ammonium bicarbonate in H₂O:MeCN) to afford tert-butyl 1-(4-aminobutyl)-6-chloro-3-(3-(naphthalen-1-yloxy)propyl)-7-(1,3,5-trimethyl-1H-pyrazol-4-yl)-1H-indole-2-carboxylate (55 mg, 0.086 mmol, 24% over 2 steps) as white solid.

LCMS (ESI+): m/z 633.4 [M+H]⁺

### Step C

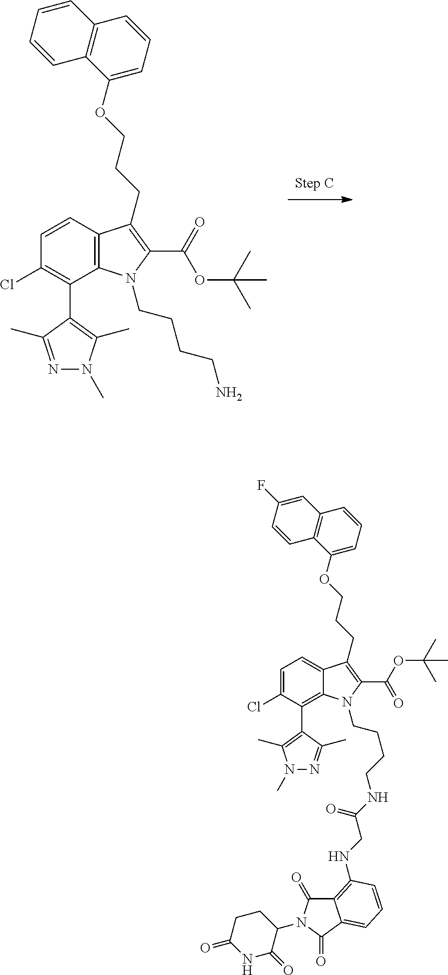 [SMILES: Cc1nn(C)c(C)c1-c1c(Cl)ccc2c(CCCOc3cccc4ccccc34)c(C(=O)OC(C)(C)C)n(CCCCN)c12 ; Cc1nn(C)c(C)c1-c1c(Cl)ccc2c(CCCOc3cccc4cc(F)ccc34)c(C(=O)OC(C)(C)C)n(CCCCNC(=O)CNc3cccc4c3C(=O)N(C3CCC(=O)NC3=O)C4=O)c12]

To a solution of tert-butyl 1-(4-aminobutyl)-6-chloro-3-(3-(naphthalen-1-yloxy)propyl)-7-(1,3,5-trimethyl-1H-pyrazol-4-yl)-1H-indole-2-carboxylate (20.0 mg, 0.032 mmol), 2-{[2-(2,6-dioxopiperidin-3-yl)-1,3-dioxo-2,3-dihydro-1H-isoindol-4-yl]amino}acetic acid (12.6 mg, 0.038 mmol) and HATU (24.0 mg, 0.063 mmol) in dry DMF (2.0 mL) was added DIPEA (0.017 mL, 0.095 mmol). The mixture was stirred at room temperature for 30 min. The crude was diluted with DCM and washed with brine. The organic layer was dried over Na₂SO₄, filtered and concentrated in vacuo to get 45 mg crude of tert-butyl 6-chloro-1-[4-(2-{[2-(2,6-dioxopiperidin-3-yl)-1,3-dioxo-2,3-dihydro-1H-isoindol-4-yl]amino}acetamido)butyl]-3-{3-[(6-fluoronaphthalen-1-yl)oxy]propyl}-7-(1,3,5-trimethyl-1H-pyrazol-4-yl)-1H-indole-2-carboxylate as yellow solid, which was used in the next step without further purification.

LCMS (ESI+): m/z 946.3 [M+H]⁺

### Step D

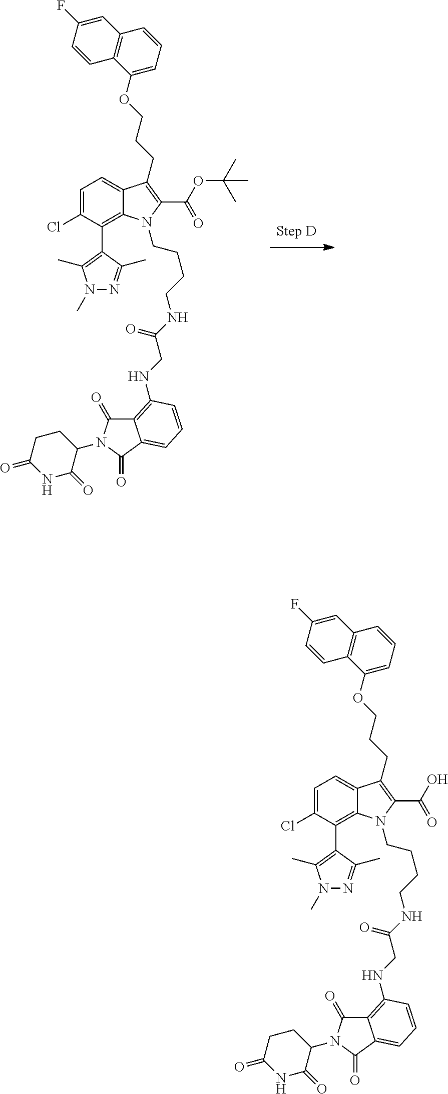 [SMILES: Cc1nn(C)c(C)c1-c1c(Cl)ccc2c(CCCOc3cccc4cc(F)ccc34)c(C(=O)OC(C)(C)C)n(CCCCNC(=O)CNc3cccc4c3C(=O)N(C3CCC(=O)NC3=O)C4=O)c12 ; Cc1nn(C)c(C)c1-c1c(Cl)ccc2c(CCCOc3cccc4cc(F)ccc34)c(C(=O)O)n(CCCCNC(=O)CNc3cccc4c3C(=O)N(C3CCC(=O)NC3=O)C4=O)c12]

To a solution of tert-butyl 6-chloro-1-[4-(2-{[2-(2,6-dioxopiperidin-3-yl)-1,3-dioxo-2,3-dihydro-1H-isoindol-4-yl]amino}acetamido)butyl]-3-{3-[(6-fluoronaphthalen-1-yl)oxy]propyl}-7-(1,3,5-trimethyl-1H-pyrazol-4-yl)-1H-indole-2-carboxylate (45 mg, crude) in DCM (1.0 mL) was added TFA (1.0 mL, 13.059 mmol). The mixture was stirred for 18 h at RT. The crude was concentrated in vacuo and purified by preparative HPLC (H₂O:MeCN+0.1% FA) to obtain 6-chloro-1-[4-(2-{[2-(2,6-dioxopiperidin-3-yl)-1,3-dioxo-2,3-dihydro-1H-isoindol-4-yl]amino}acetamido)butyl]-3-{3-[(6-fluoronaphthalen-1-yl)oxy]propyl}-7-(1,3,5-trimethyl-1H-pyrazol-4-yl)-1H-indole-2-carboxylic acid (12.7 mg, 0.014 mmol, 44% over two steps) as a white solid.

LCMS (ESI+): m/z 890.3 [M+H]⁺

¹H NMR (500 MHz, DMSO, 353 K) δ 10.77 (s, 1H), 8.25 (dd, J=9.3, 5.9 Hz, 1H), 7.76-7.69 (m, 2H), 7.58 (dd, J=10.3, 2.6 Hz, 1H), 7.54 (dd, J=8.5, 7.1 Hz, 1H), 7.44-7.41 (m, 2H), 7.33 (td, J=8.9, 2.6 Hz, 1H), 7.20 (d, J=8.5 Hz, 1H), 7.05 (d, J=7.1 Hz, 1H), 6.90-6.81 (m, 3H), 5.02 (dd, J=12.6, 5.5 Hz, 1H), 4.24 (t, J=6.2 Hz, 2H), 4.15 (ddd, J=15.3, 10.0, 5.6 Hz, 1H), 3.99 (ddd, J=14.2, 9.9, 5.2 Hz, 1H), 3.88 (d, J=5.6 Hz, 2H), 3.75 (s, 3H), 3.29 (dd, J=8.6, 6.5 Hz, 2H), 2.66-2.52 (m, 2H), 2.26-2.19 (m, 2H), 2.09-2.03 (m, 1H), 1.99 (s, 3H), 1.87 (s, 3H), 1.26-1.19 (m, 1H), 1.19-1.09 (m, 1H), 1.02-0.94 (m, 2H).

3 protons in aliphatic area overlaps with water.

### Example 95. 6-Chloro-1-[4-(2-{[2-(2,6-dioxopiperidin-3-yl)-1-oxo-2,3-dihydro-1H-isoindol-4-yl]oxy}-N-methylacetamido)butyl]-3-{3-[(6-fluoronaphthalen-1-yl)oxy]propyl}-7-(1,3,5-trimethyl-1H-pyrazol-4-yl)-1H-indole-2-carboxylic acid (326)

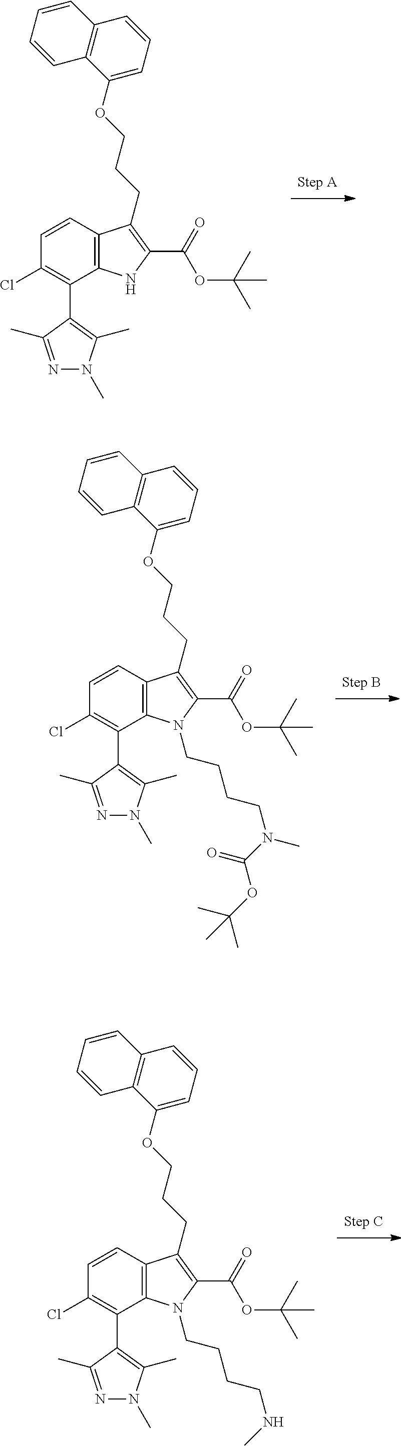

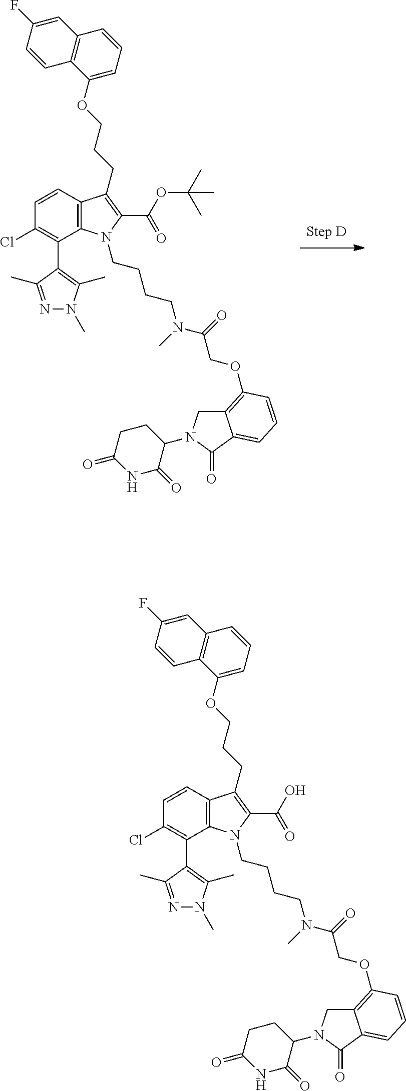 [SMILES: Cc1nn(C)c(C)c1-c1c(Cl)ccc2c(CCCOc3cccc4cc(F)ccc34)c(C(=O)OC(C)(C)C)n(CCCCN(C)C(=O)COc3cccc4c3CN(C3CCC(=O)NC3=O)C4=O)c12 ; Cc1nn(C)c(C)c1-c1c(Cl)ccc2c(CCCOc3cccc4cc(F)ccc34)c(C(=O)O)n(CCCCN(C)C(=O)COc3cccc4c3CN(C3CCC(=O)NC3=O)C4=O)c12]

### Step A

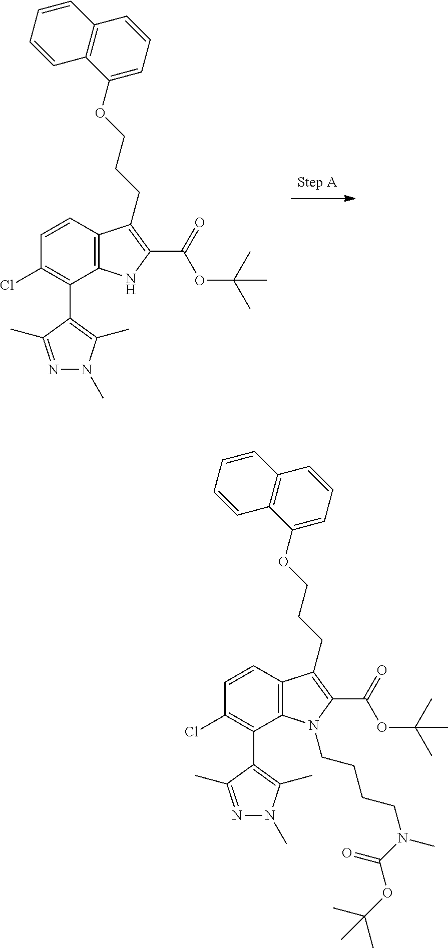 [SMILES: Cc1nn(C)c(C)c1-c1c(Cl)ccc2c(CCCOc3cccc4ccccc34)c(C(=O)OC(C)(C)C)[nH]c12 ; Cc1nn(C)c(C)c1-c1c(Cl)ccc2c(CCCOc3cccc4ccccc34)c(C(=O)OC(C)(C)C)n(CCCCN(C)C(=O)OC(C)(C)C)c12]

To a well stirred solution of tert-butyl 6-chloro-3-(3-(naphthalen-1-yloxy)propyl)-7-(1,3,5-trimethyl-1H-pyrazol-4-yl)-1H-indole-2-carboxylate (200 mg, 0.357 mmol) in DMF (3 mL) was added tert-butyl (4-bromobutyl)(methyl)carbamate (283 mg, 1.07 mmol) followed by Cs₂CO₃ (289 mg, 0.891 mmol) and the mixture was allowed to stir at 90° C. for 16 h under nitrogen. After complete consumption of the starting material the reaction mixture was diluted with EtOAc, washed successively with water and brine, the organic layer was dried over Na₂SO₄ and evaporated under reduced pressure to get the crude compound, which was then purified by column chromatography (SiO₂, 60% EtOAc in DCM) to get tert-butyl 1-(4-((tert-butoxycarbonyl)(methyl)amino)butyl)-6-chloro-3-(3-((6-fluoronaphthalen-1-yl)oxy)propyl)-7-(1,3,5-trimethyl-1H-pyrazol-4-yl)-1H-indole-2-carboxylate (160 mg, 0.214 mmol, 60%) of as gummy solid.

### Step B

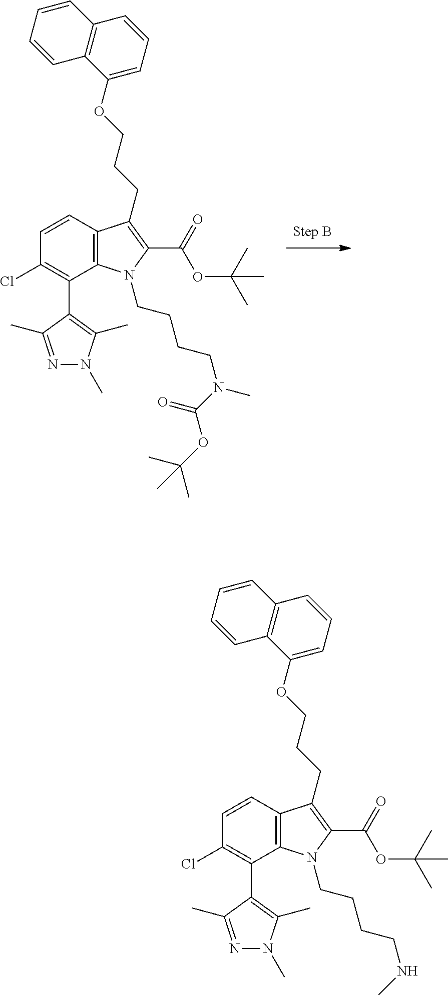 [SMILES: Cc1nn(C)c(C)c1-c1c(Cl)ccc2c(CCCOc3cccc4ccccc34)c(C(=O)OC(C)(C)C)n(CCCCN(C)C(=O)OC(C)(C)C)c12 ; CNCCCCn1c(C(=O)OC(C)(C)C)c(CCCOc2cccc3ccccc23)c2ccc(Cl)c(-c3c(C)nn(C)c3C)c21]

tert-Butyl 1-(4-((tert-butoxycarbonyl)(methyl)amino)butyl)-6-chloro-3-(3-((6-fluoronaphthalen-1-yl)oxy)propyl)-7-(1,3,5-trimethyl-1H-pyrazol-4-yl)-1H-indole-2-carboxylate (160 mg, 0.214 mmol) dissolved in DCM followed by addition of 4 M HCl in dioxane (5 mL) at 0° C. and the mixture stirred for 2 h under nitrogen at same temperature. Where upon LCMS indicated the reaction was completed, to the reaction mixture was added dropwise cold 1M NaOH solution at 0° C. to maintain the pH˜5 to 6. The aqueous layer was extracted 2 times with DCM. The combined organics were dried over Na₂SO₄ and concentrated in vacuo to get the crude compound which was purified by preparative HPLC to get tert-butyl 6-chloro-1-(4-(methylamino)butyl)-3-(3-(naphthalen-1-yloxy)propyl)-7-(1,3,5-trimethyl-1H-pyrazol-4-yl)-1H-indole-2-carboxylate (26 mg, 0.04 mmol, 18.7%).

LCMS (ESI+): m/z 647.5 [M+H]⁺

### Step C

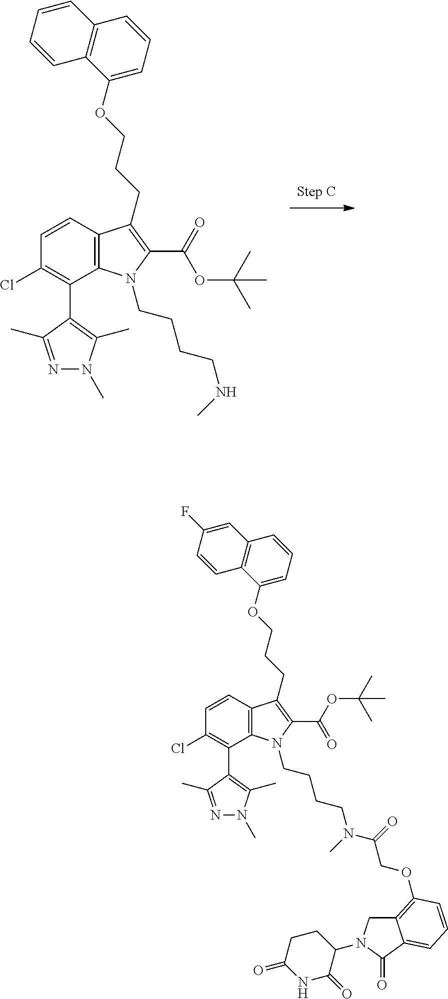 [SMILES: CNCCCCn1c(C(=O)OC(C)(C)C)c(CCCOc2cccc3ccccc23)c2ccc(Cl)c(-c3c(C)nn(C)c3C)c21 ; Cc1nn(C)c(C)c1-c1c(Cl)ccc2c(CCCOc3cccc4cc(F)ccc34)c(C(=O)OC(C)(C)C)n(CCCCN(C)C(=O)COc3cccc4c3CN(C3CCC(=O)NC3=O)C4=O)c12]

To a solution of tert-butyl 6-chloro-3-{3-[(6-fluoronaphthalen-1-yl)oxy]propyl}-1-[4-(methylamino)butyl]-7-(1,3,5-trimethyl-1H-pyrazol-4-yl)-1H-indole-2-carboxylate (23.5 mg, 0.036 mmol), 2-{[2-(2,6-dioxopiperidin-3-yl)-1-oxo-2,3-dihydro-1H-isoindol-4-yl]oxy}acetic acid (13.9 mg, 0.044 mmol) and HATU (27.6 mg, 0.073 mmol) in dry DMF (2.0 mL) was added DIPEA (0.019 mL, 0.109 mmol). The mixture was stirred at room temperature for 30 min. The crude was diluted with DCM and washed with brine. The organic layer was dried over Na₂SO₄, filtered and concentrated in vacuo to get 68 mg crude of tert-butyl 6-chloro-1-[4-(2-{[2-(2,6-dioxopiperidin-3-yl)-1-oxo-2,3-dihydro-1H-isoindol-4-yl]oxy}-N-methylacetamido)butyl]-3-{3-[(6-fluoronaphthalen-1-yl)oxy]propyl}-7-(1,3,5-trimethyl-1H-pyrazol-4-yl)-1H-indole-2-carboxylate as orange oil which was used in the next step without further purification.

LCMS (ESI+): m/z 947.3 [M+H]⁺

### Step D

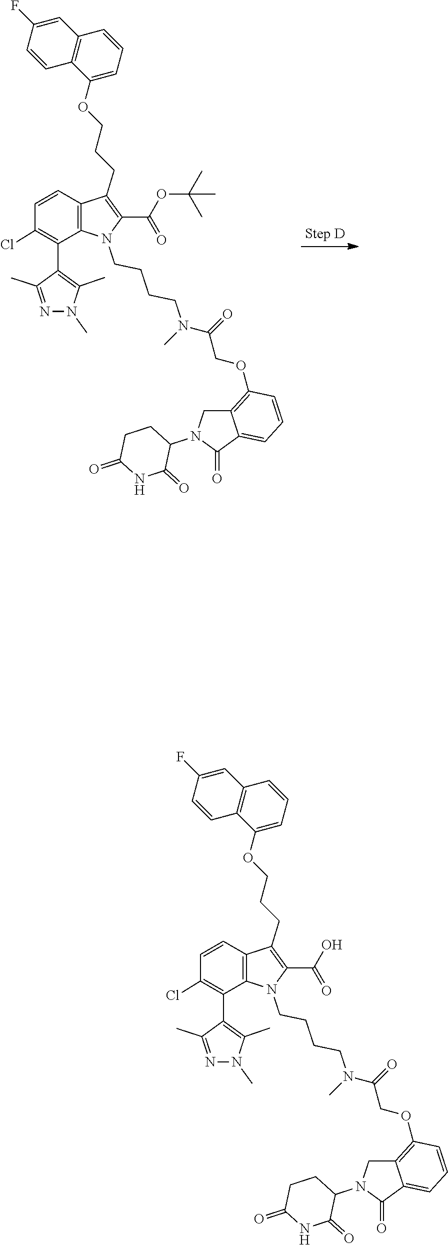 [SMILES: Cc1nn(C)c(C)c1-c1c(Cl)ccc2c(CCCOc3cccc4cc(F)ccc34)c(C(=O)OC(C)(C)C)n(CCCCN(C)C(=O)COc3cccc4c3CN(C3CCC(=O)NC3=O)C4=O)c12 ; Cc1nn(C)c(C)c1-c1c(Cl)ccc2c(CCCOc3cccc4cc(F)ccc34)c(C(=O)O)n(CCCCN(C)C(=O)COc3cccc4c3CN(C3CCC(=O)NC3=O)C4=O)c12]

To a solution of tert-butyl 6-chloro-1-[4-(2-{[2-(2,6-dioxopiperidin-3-yl)-1-oxo-2,3-dihydro-1H-isoindol-4-yl]oxy}-N-methylacetamido)butyl]-3-{3-[(6-fluoronaphthalen-1-yl)oxy]propyl}-7-(1,3,5-trimethyl-1H-pyrazol-4-yl)-1H-indole-2-carboxylate (68 mg, crude) in DCM (1.0 mL) was added TFA (1.0 mL, 13.059 mmol). The mixture was stirred for 18 h at RT. The crude was concentrated in vacuo and purified by preparative HPLC (C18, H₂O:MeCN+0.1% FA) to obtain 6-chloro-1-[4-(2-{[2-(2,6-dioxopiperidin-3-yl)-1-oxo-2,3-dihydro-1H-isoindol-4-yl]oxy}-N-methylacetamido)butyl]-3-{3-[(6-fluoronaphthalen-1-yl)oxy]propyl}-7-(1,3,5-trimethyl-1H-pyrazol-4-yl)-1H-indole-2-carboxylic acid (16.7 mg, 0.019 mmol, 53% over two steps) as a white solid.

LCMS (ESI+): m/z 891.3 [M+H]⁺

¹H NMR (500 MHz, DMSO) δ 13.28 (s, 1H), 10.96 (s, 1H), 8.25 (dd, J=9.2, 5.9 Hz, 1H), 7.73 (d, J=8.5 Hz, 1H), 7.65 (dd, J=10.4, 2.6 Hz, 1H), 7.47-7.40 (m, 2H), 7.38 (tdd, J=9.2, 6.2, 2.7 Hz, 2H), 7.29 (dt, J=7.6, 2.1 Hz, 1H), 7.21 (dd, J=8.6, 4.3 Hz, 1H), 7.06 (dt, J=8.4, 3.2 Hz, 1H), 6.88 (dd, J=5.3, 3.4 Hz, 1H), 5.14-5.06 (m, 1H), 5.00-4.85 (m, 2H), 4.38 (dd, J=17.5, 2.7, 1H), 4.25 (dd, J=17.5, 3.6, 1H), 4.23-4.12 (m, 3H), 4.10-3.87 (m, 1H), 3.77-3.66 (m, 3H), 3.16-3.03 (m, 2H), 3.01-2.85 (m, 4H), 2.62-2.50 (m, 1H), 2.43 (td, J=13.3, 4.6 Hz, 1H), 2.19 (p, J=6.3 Hz, 2H), 2.05-1.93 (m, 4H), 1.90-1.82 (m, 3H), 1.22-1.09 (m, 2H), 1.08-0.94 (m, 2H).

2 protons in aliphatic area overlap with water.

### Example 96. 6-Chloro-1-(2-((2-(2-((2-(2,6-dioxopiperidin-3-yl)-1-oxoisoindolin-4-yl)oxy)-N-methylacetamido)ethyl)(methyl)amino)ethyl)-3-(3-((6-fluoronaphthalen-1-yl)oxy)propyl)-7-(1,3,5-trimethyl-1H-pyrazol-4-yl)-1H-indole-2-carboxylic acid (327)

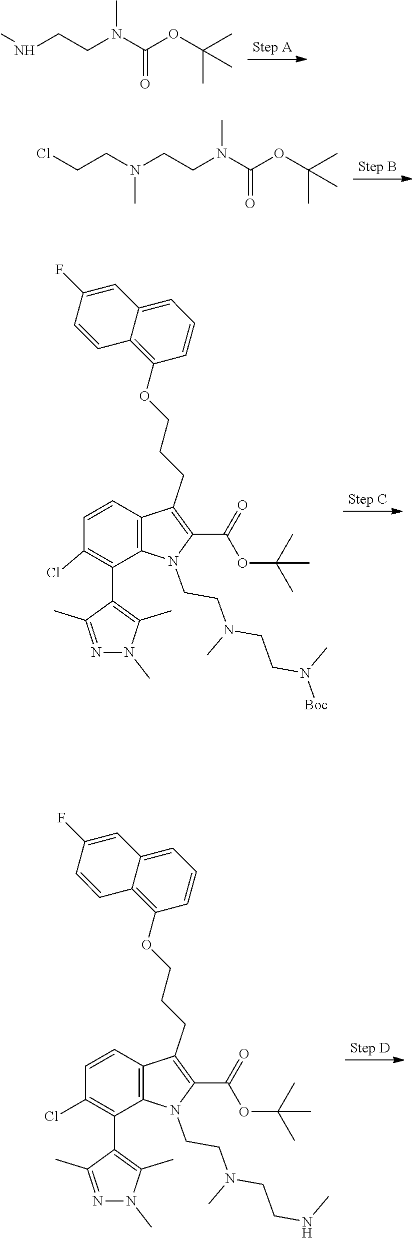

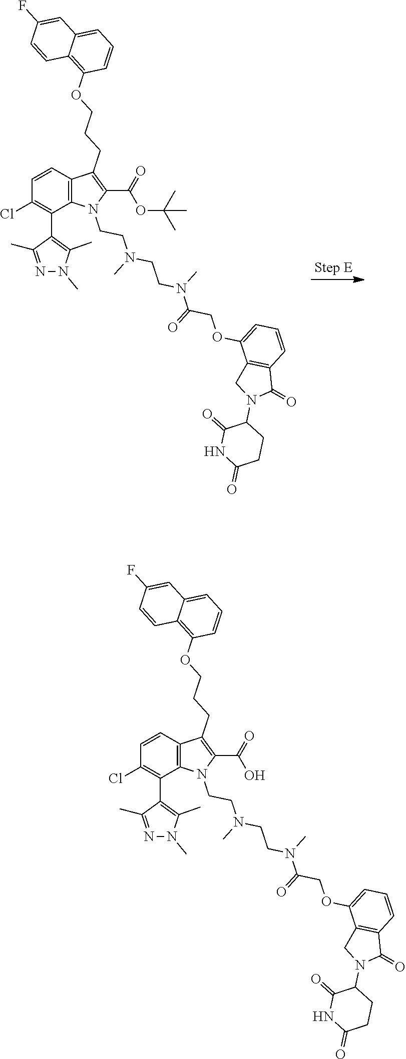 [SMILES: Cc1nn(C)c(C)c1-c1c(Cl)ccc2c(CCCOc3cccc4cc(F)ccc34)c(C(=O)OC(C)(C)C)n(CCN(C)CCN(C)C(=O)COc3cccc4c3CN(C3CCC(=O)NC3=O)C4=O)c12 ; Cc1nn(C)c(C)c1-c1c(Cl)ccc2c(CCCOc3cccc4cc(F)ccc34)c(C(=O)O)n(CCN(C)CCN(C)C(=O)COc3cccc4c3CN(C3CCC(=O)NC3=O)C4=O)c12]

### Step A

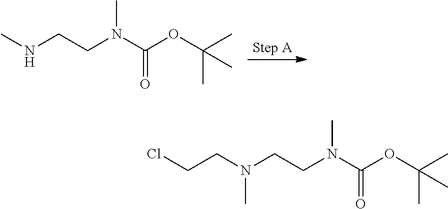 [SMILES: CNCCN(C)C(=O)OC(C)(C)C ; CN(CCCl)CCN(C)C(=O)OC(C)(C)C]

To a solution of tert-butyl methyl[2-(methylamino)ethyl]carbamate (700 mg, 3.72 mmol) in DMF (15 mL) at 0° C. was added NaH (144 mg, 6.00 mmol) at 0° C. portion wise under nitrogen and the reaction mixture was allowed to stir at the same temperature for 1 h. After that time, to the reaction mixture was added solution of 1-bromo-2-chloroethane (2.1 g, 14.6 mmol) in DMF (5 mL) dropwise at 0° C. and the reaction mixture was allowed to stir at room temperature for 3 h under nitrogen. After complete consumption of the starting material, the reaction mixture was again cooled to 0° C. and the excess of NaH was quenched with NH₄Cl solution. The reaction mass was then diluted with EtOAc, washed successively with water and brine. The organic layer was dried over Na₂SO₄ and evaporated under reduced pressure. The crude was purified by column chromatography (SiO₂, 30% EtOAc in hexane) to get tert-butyl (2-((2-chloroethyl)(methyl)amino)ethyl)(methyl)carbamate (400 mg, 1.6 mmol, 43%) as light yellow dense liquid.

LCMS (ESI+): m/z 251.2 [M+H]⁺

### Step B

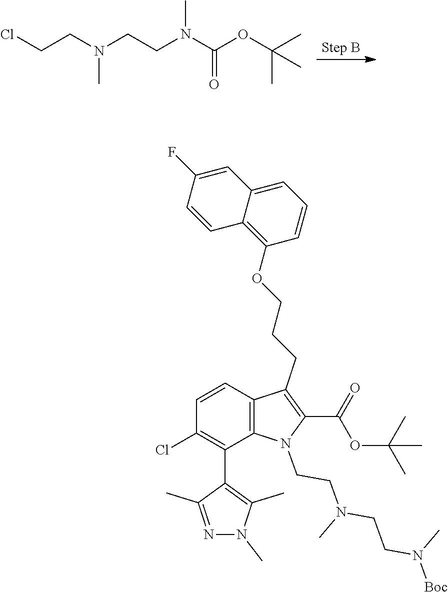

To a well stirred solution of tert-butyl 6-chloro-3-(3-((6-fluoronaphthalen-1-yl)oxy)propyl)-7-(1,3,5-trimethyl-1H-pyrazol-4-yl)-1H-indole-2-carboxylate (300 mg, 0.53 mmol) in DMF (3 mL) was added tert-butyl (2-((2-chloroethyl)(methyl)amino)ethyl)(methyl)carbamate (267 mg, 1.07 mmol) followed by Cs₂CO₃ (292 mg, 0.90 mmol) and the mixture was allowed to stir at 90° C. for 16 h under nitrogen. After complete consumption of the starting material, the reaction mixture was diluted with EtOAc and washed successively with water and brine. The organic layer was dried over Na₂SO₄ and evaporated under reduced pressure to get the crude compound, which was then purified by column chromatography (SiO₂, 60% EtOAc in DCM) to get of tert-butyl 1-(2-((2-((tert-butoxycarbonyl)(methyl)amino)ethyl)(methyl)amino)ethyl)-6-chloro-3-(3-((6-fluoronaphthalen-1-yl)oxy)propyl)-7-(1,3,5-trimethyl-1H-pyrazol-4-yl)-1H-indole-2-carboxylate (350 mg, 0.45 mmol, 84%) as gummy solid.

LCMS (ESI+): m/z 776.2 [M+H]⁺

### Step C

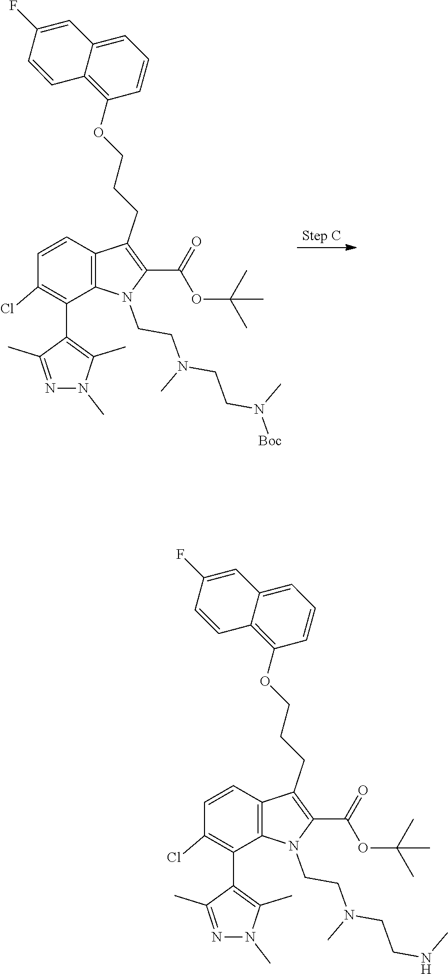

tert-Butyl 1-(2-((2-((tert-butoxycarbonyl)(methyl)amino)ethyl)(methyl)amino)ethyl)-6-chloro-3-(3-((6-fluoronaphthalen-1-yl)oxy)propyl)-7-(1,3,5-trimethyl-1H-pyrazol-4-yl)-1H-indole-2-carboxylate (350 mg, 0.45 mmol) was dissolved in DCM (10 mL), followed by the addition of 4M HCl in dioxane (5 mL) at 0° C. and the mixture was stirred for 2 h under nitrogen at same temperature. After complete consumption of the starting material, to the reaction mixture was added dropwise cold 1M NaOH solution at 0° C. to maintain the pH ˜5 to 6. The aqueous layer was extracted 2-3 times with DCM. The combined organics were dried over Na₂SO₄ and concentrated in vacuo to get the crude compound which was purified by reverse phase preparative HPLC (C18, 20 mM ammonium bicarbonate buffer in H₂O:MeCN) to get tert-butyl 6-chloro-3-(3-((6-fluoronaphthalen-1-yl)oxy)propyl)-1-(2-(methyl(2-(methylamino)ethyl)amino)ethyl)-7-(1,3,5-trimethyl-1H-pyrazol-4-yl)-1H-indole-2-carboxylate (180 mg, 0.266 mmol, 59%).

LCMS (ESI+): m/z 676.3 [M+H]⁺

### Step D

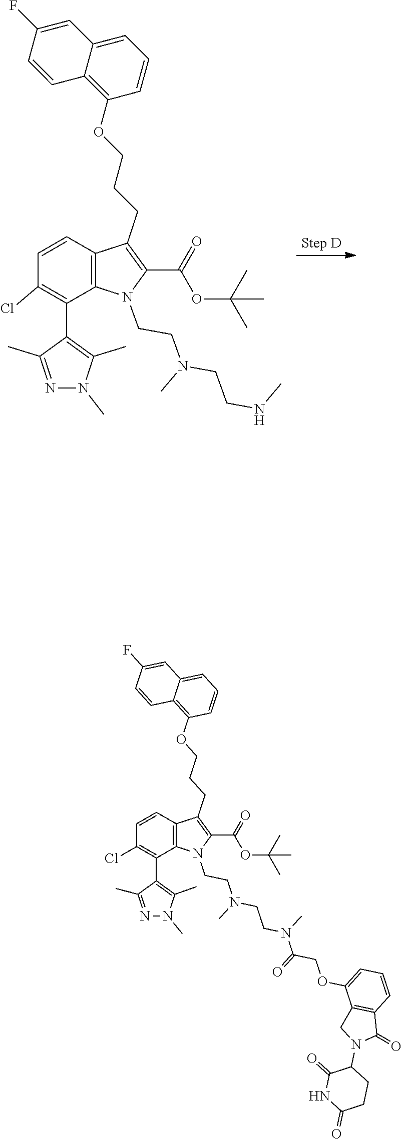

2-{[2-(2,6-Dioxopiperidin-3-yl)-1-oxo-2,3-dihydro-1H-isoindol-4-yl]oxy}acetic acid (20.7 mg, 0.065 mmol) was dissolved in dry DMF (2.4 mL) under argon atmosphere and HATU (23.6 mg, 0.062 mmol), followed by DIPEA (0.031 mL, 0.177 mmol) were added. The solution was stirred for 15 min at room temperature and to it, tert-butyl 6-chloro-3-{3-[(6-fluoronaphthalen-1-yl)oxy]propyl}-1-(2-{methyl[2-(methylamino)ethyl]amino}ethyl)-7-(1,3,5-trimethyl-1H-pyrazol-4-yl)-1H-indole-2-carboxylate (40.0 mg, 0.059 mmol) was added. The reaction was stirred for 20 min. After complete consumption of the starting material, the solution was diluted with DCM and washed with brine and water. The organic layer was dried over anhydrous MgSO₄, filtered and dried under reduced pressure to obtain tert-butyl 6-chloro-1-(2-((2-(2-((2-(2,6-dioxopiperidin-3-yl)-1-oxoisoindolin-4-yl)oxy)-N-methylacetamido)ethyl)(methyl)amino)ethyl)-3-(3-((6-fluoronaphthalen-1-yl)oxy)propyl)-7-(1,3,5-trimethyl-1H-pyrazol-4-yl)-1H-indole-2-carboxylate (74.8 mg, crude) was obtained as yellow oil and used in the next step without further purification.

LCMS (ESI+): m/z 975.8 [M+H]⁺

### Step E

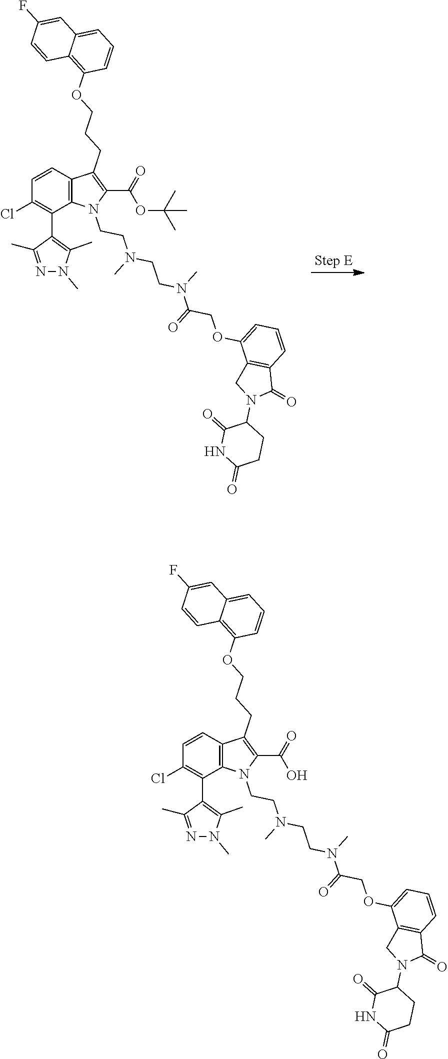 [SMILES: Cc1nn(C)c(C)c1-c1c(Cl)ccc2c(CCCOc3cccc4cc(F)ccc34)c(C(=O)OC(C)(C)C)n(CCN(C)CCN(C)C(=O)COc3cccc4c3CN(C3CCC(=O)NC3=O)C4=O)c12 ; Cc1nn(C)c(C)c1-c1c(Cl)ccc2c(CCCOc3cccc4cc(F)ccc34)c(C(=O)O)n(CCN(C)CCN(C)C(=O)COc3cccc4c3CN(C3CCC(=O)NC3=O)C4=O)c12]

tert-Butyl 6-chloro-1-(2-((2-(2-((2-(2,6-dioxopiperidin-3-yl)-1-oxoisoindolin-4-yl)oxy)-N-methylacetamido)ethyl)(methyl)amino)ethyl)-3-(3-((6-fluoronaphthalen-1-yl)oxy)propyl)-7-(1,3,5-trimethyl-1H-pyrazol-4-yl)-1H-indole-2-carboxylate (74.8 mg, crude) was dissolved in dry DCM (0.75 mL) under argon atmosphere and TFA (0.748 mL) was added. The reaction mixture was stirred for 16 h at room temperature. After complete consumption of the starting material, the reaction mixture was dried under reduced pressure. The resulting residue was dissolved in DMSO and purified with preparative HPLC (C18, H₂O:MeCN+0.1% FA) to give 6-chloro-1-(2-((2-(2-((2-(2,6-dioxopiperidin-3-yl)-1-oxoisoindolin-4-yl)oxy)-N-methylacetamido)ethyl)(methyl)amino)ethyl)-3-(3-((6-fluoronaphthalen-1-yl)oxy)propyl)-7-(1,3,5-trimethyl-1H-pyrazol-4-yl)-1H-indole-2-carboxylic acid (36.9 mg, 0.040 mmol, 67.8% over 2 steps) as white solid.

LCMS (ESI+): m/z 920.7 [M+H]⁺

¹H NMR (500 MHz, DMSO, 353 K) δ 10.65 (s, 1H), 8.24 (dd, J=9.3, 5.9 Hz, 1H), 7.70 (d, J=8.5 Hz, 1H), 7.58 (dd, J=10.4, 2.6 Hz, 1H), 7.46-7.37 (m, 3H), 7.36-7.29 (m, 2H), 7.21 (d, J=8.5 Hz, 1H), 7.12 (d, J=8.1 Hz, 1H), 6.87 (dd, J=5.8, 2.9 Hz, 1H), 5.05 (dd, J=13.1, 5.2 Hz, 1H), 4.89 (s, 2H), 4.40 (d, J=17.1 Hz, 1H), 4.32 (d, J=17.1 Hz, 1H), 4.29-4.15 (m, 4H), 3.73 (s, 3H), 3.30-3.26 (m, 2H), 3.25-3.21 (m, 2H), 2.90-2.85 (m, 3H), 2.79-2.71 (m, 1H), 2.65-2.59 (m, 1H), 2.43 (qd, J=13.0, 4.6 Hz, 1H), 2.38-2.29 (m, 2H), 2.29-2.15 (m, 4H), 2.08-1.97 (m, 7H), 1.89 (s, 3H).

### Example 97. 6-Chloro-1-(5-(2-((2-(2,6-dioxopiperidin-3-yl)-1-oxoisoindolin-4-yl)oxy)-N-methylacetamido)pentyl)-3-(3-((6-fluoronaphthalen-1-yl)oxy)propyl)-7-(1,3,5-trimethyl-1H-pyrazol-4-yl)-1H-indole-2-carboxylic acid (328)

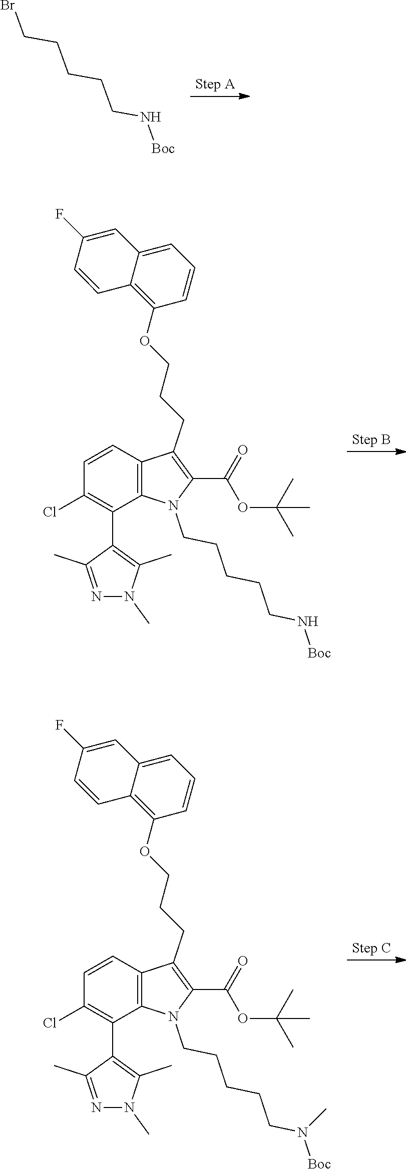

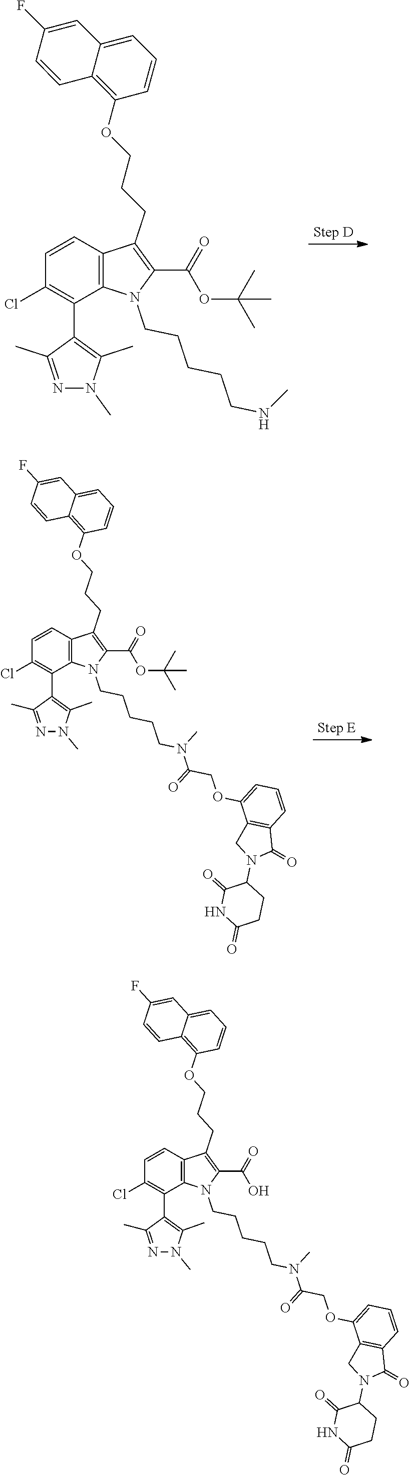

### Step A

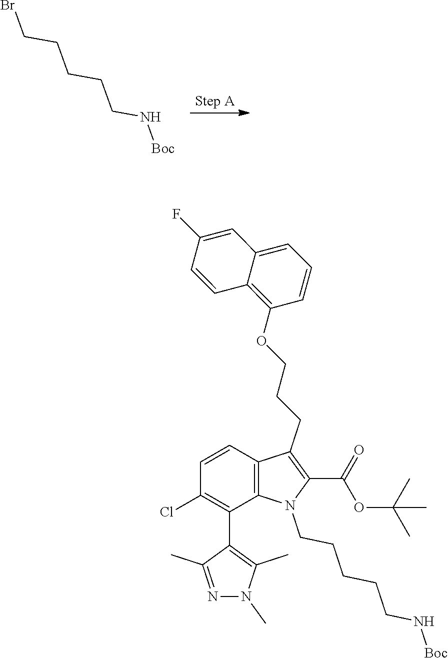 [SMILES: CC(C)(C)OC(=O)NCCCCCBr ; Cc1nn(C)c(C)c1-c1c(Cl)ccc2c(CCCOc3cccc4cc(F)ccc34)c(C(=O)OC(C)(C)C)n(CCCCCNC(=O)OC(C)(C)C)c12]

To a solution of tert-butyl 6-chloro-3-(3-((6-fluoronaphthalen-1-yl)oxy)propyl)-7-(1,3,5-trimethyl-1H-pyrazol-4-yl)-1H-indole-2-carboxylate (400 mg, 0.71 mmol) in DMF (12 mL) was added tert-butyl (5-bromopentyl)carbamate (566 mg, 2.13 mmol) followed by Cs₂CO₃ (695 mg, 2.13 mmol) and the mixture was allowed to stir at 90° C. for 16 h under nitrogen. After complete consumption of the starting material, the reaction mixture was diluted with EtOAc, washed successively with water and brine. The organic layer was dried over Na₂SO₄ and evaporated under reduced pressure to get tert-butyl 1-(5-((tert-butoxycarbonyl)amino)pentyl)-6-chloro-3-(3-((6-fluoronaphthalen-1-yl)oxy)propyl)-7-(1,3,5-trimethyl-1H-pyrazol-4-yl)-1H-indole-2-carboxylate (350 mg, crude), which was then directly used in the next step without further purification.

LCMS (ESI+): m/z 747.4 [M+H]⁺

### Step B

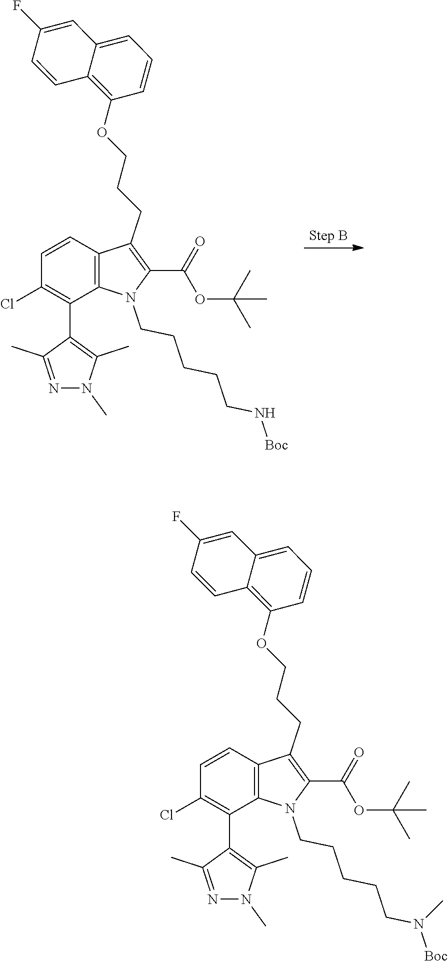

To a stirred solution of tert-butyl 1-(5-((tert-butoxycarbonyl)amino)pentyl)-6-chloro-3-(3-((6-fluoronaphthalen-1-yl)oxy)propyl)-7-(1,3,5-trimethyl-1H-pyrazol-4-yl)-1H-indole-2-carboxylate (350 mg, crude) in DMF (3 mL) was added dry NaH (22.5 mg, 0.94 mmol) portion wise at 0° C. under nitrogen. The reaction mixture was allowed to stir at room temperature for 30 minutes after that the reaction mixture was again cooled to 0° C. and was added Mel (0.30 mL, 4.7 mmol) dropwise. The resulting reaction mixture was allowed to stir at room temperature under nitrogen for 3 h. After complete consumption of the starting material, the excess NaH was quenched by addition of ice water and diluted with EtOAc. The organic layer was washed with ice water and brine, dried over Na₂SO₄ and then evaporated under reduced pressure to get the crude tert-butyl 1-(5-((tert-butoxycarbonyl)(methyl)amino)pentyl)-6-chloro-3-(3-((6-fluoronaphthalen-1-yl)oxy)propyl)-7-(1,3,5-trimethyl-1H-pyrazol-4-yl)-1H-indole-2-carboxylate (300 mg), which was then directly used in the next step without further purification.

LCMS (ESI+): m/z 761.4 [M+H]⁺

### Step C

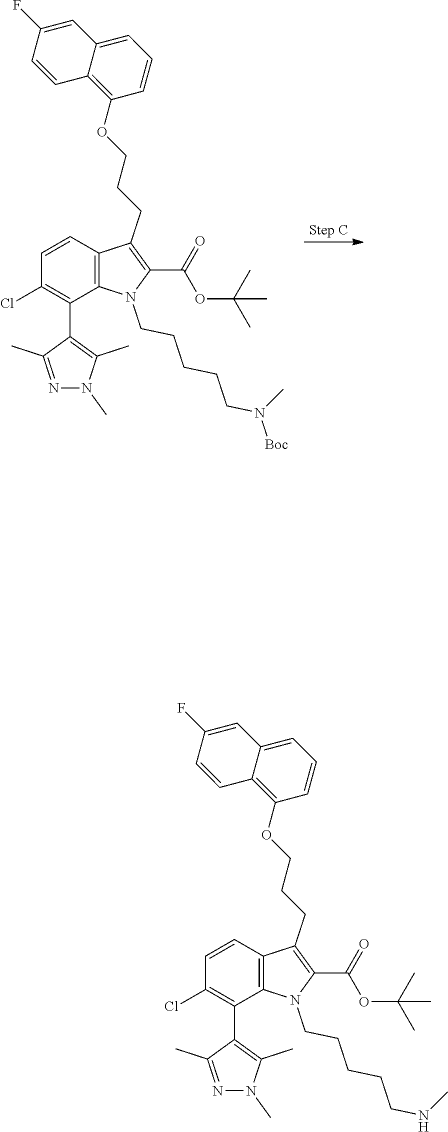

To a solution of tert-butyl 1-(5-((tert-butoxycarbonyl)(methyl)amino)pentyl)-6-chloro-3-(3-((6-fluoronaphthalen-1-yl)oxy)propyl)-7-(1,3,5-trimethyl-1H-pyrazol-4-yl)-1H-indole-2-carboxylate (300 mg, crude) in dioxane (3 mL), 4M HCl in dioxane (12 mL) was added at 0° C. and the mixture was stirred for 2 h under nitrogen at same temperature. Where upon LCMS indicated the reaction was completed, to the reaction mixture was added dropwise cold 1M NaOH solution at 0° C. to pH ˜5 to 6. The aqueous layer was extracted 2-3 times with DCM. The combined organics were dried over Na₂SO₄ and concentrated in vacuo. The crude compound was purified by reverse phase column chromatography (10 mM ammonium acetate aqueous buffer:MeCN) to get tert-butyl 6-chloro-3-(3-((6-fluoronaphthalen-1-yl)oxy)propyl)-1-(5-(methylamino)pentyl)-7-(1,3,5-trimethyl-1H-pyrazol-4-yl)-1H-indole-2-carboxylate (85 mg, 0.128 mmol, 18% over 3 steps) as light brown solid.

LCMS (ESI+): m/z 661.45 [M+H]⁺

### Step D

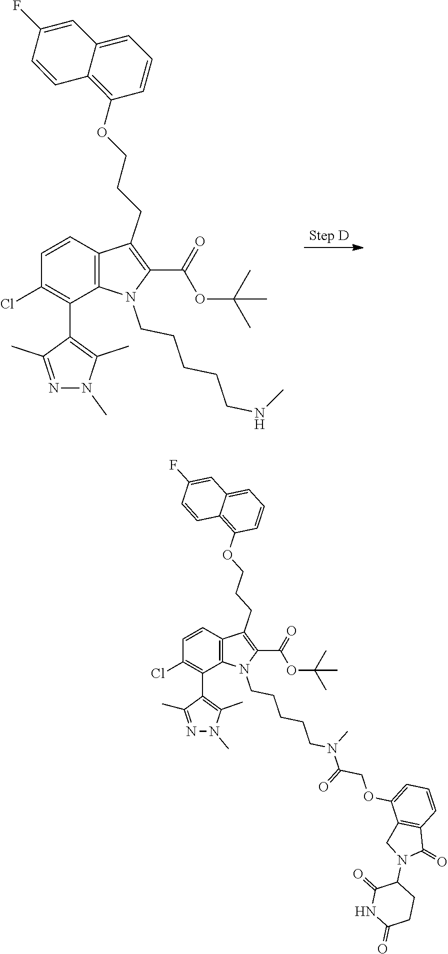

2-{[2-(2,6-Dioxopiperidin-3-yl)-1-oxo-2,3-dihydro-1H-isoindol-4-yl]oxy}acetic acid (5.3 mg, 0.017 mmol) was dissolved in dry DMF (0.756 mL) under argon atmosphere and HATU (6.0 mg, 0.016 mmol), followed by DIPEA (0.008 mL, 0.045 mmol) was added. Reaction was stirred for 15 min at room temperature and tert-butyl 6-chloro-3-(3-((6-fluoronaphthalen-1-yl)oxy)propyl)-1-(5-(methylamino)pentyl)-7-(1,3,5-trimethyl-1H-pyrazol-4-yl)-1H-indole-2-carboxylate (10.0 mg, 0.015 mmol) was added. Reaction was stirred under argon at room temperature for next 20 min. After complete consumption of the starting material, the mixture was dissolved with DCM and washed with brine and water. Organic layer was dried over anhydrous MgSO₄, filtered and dried under reduced pressure to obtain 14.5 mg crude tert-butyl 6-chloro-1-(5-(2-((2-(2,6-dioxopiperidin-3-yl)-1-oxoisoindolin-4-yl)oxy)-N-methylacetamido)pentyl)-3-(3-((6-fluoronaphthalen-1-yl)oxy)propyl)-7-(1,3,5-trimethyl-1H-pyrazol-4-yl)-1H-indole-2-carboxylate as yellow oil and used in the following step without further purification.

LCMS (ESI+): m/z 961.0 [M+H]⁺

### Step E

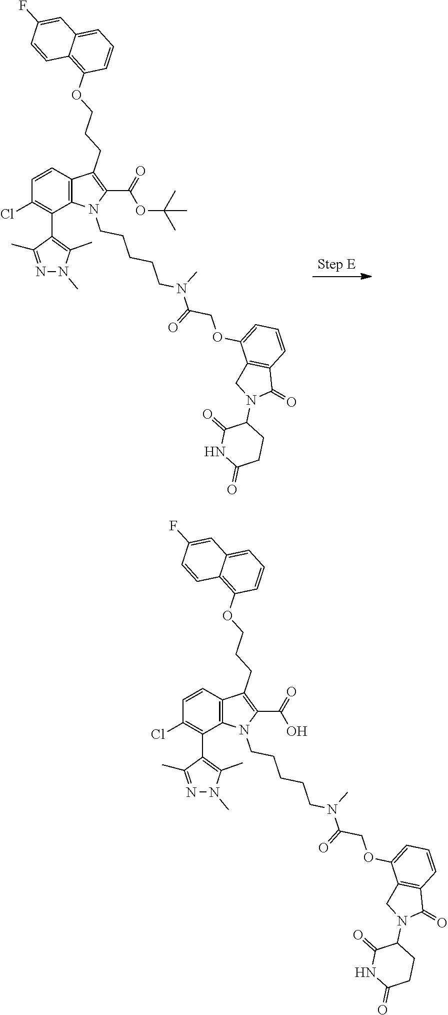

tert-Butyl 6-chloro-1-(5-(2-((2-(2,6-dioxopiperidin-3-yl)-1-oxoisoindolin-4-yl)oxy)-N-methylacetamido)pentyl)-3-(3-((6-fluoronaphthalen-1-yl)oxy)propyl)-7-(1,3,5-trimethyl-1H-pyrazol-4-yl)-1H-indole-2-carboxylate (14.5 mg, crude) was dissolved in dry DCM (0.2 mL) under argon atmosphere and TFA (0.200 mL) was added. Reaction was stirred for 16 h at room temperature. After complete consumption of the starting material, reaction mixture was concentrated under reduced pressure. Resulting residue was dissolved in DMSO (4.0 mL) and purified with preparative HPLC (C18, H₂O:MeCN+0.1% FA) to give 6-chloro-1-(5-(2-((2-(2,6-dioxopiperidin-3-yl)-1-oxoisoindolin-4-yl)oxy)-N-methylacetamido)pentyl)-3-(3-((6-fluoronaphthalen-1-yl)oxy)propyl)-7-(1,3,5-trimethyl-1H-pyrazol-4-yl)-1H-indole-2-carboxylic acid (6.1 mg, 0.007 mmol, 45% over 2 steps) as a white powder.

LCMS (ESI+): m/z 905.8 [M+H]⁺

¹H NMR (500 MHz, DMSO) δ 13.29 (s, 1H), 10.97 (s, 1H), 8.24 (dd, J=9.2, 5.9 Hz, 1H), 7.72 (d, J=8.5 Hz, 1H), 7.65 (dd, J=10.4, 2.6 Hz, 1H), 7.50-7.40 (m, 3H), 7.37 (td, J=8.9, 2.7 Hz, 1H), 7.31 (d, J=7.5 Hz, 1H), 7.20 (d, J=8.5 Hz, 1H), 7.10 (d, J=8.2 Hz, 1H), 6.88 (dd, J=5.3, 3.4 Hz, 1H), 5.11 (dd, J=13.3, 5.1 Hz, 1H), 4.96 (d, J=14.1 Hz, 2H), 4.38 (dd, J=17.2, 9.6 Hz, 1H), 4.31-4.23 (m, 1H), 4.23-4.17 (m, 2H), 4.17-3.92 (m, 2H), 3.73 (d, J=10.1 Hz, 3H), 3.29-3.25 (m, 2H), 3.20-3.11 (m, 2H), 2.94 (s, 2H), 2.93-2.86 (m, 1H), 2.78 (s, 1H), 2.62-2.55 (m, 1H), 2.47-2.41 (m, 1H), 2.24-2.13 (m, 2H), 2.04-1.92 (m, 4H), 1.89-1.81 (m, 3H), 1.36-1.07 (m, 4H), 0.91-0.64 (m, 2H).

### Example 98. 6-Chloro-1-[3-(2-{[2-(2,6-dioxopiperidin-3-yl)-1-oxo-2,3-dihydro-1H-isoindol-4-yl]oxy}ethoxy)propyl]-3-{3-[(6-fluoronaphthalen-1-yl)oxy]propyl}-7-(1,3,5-trimethyl-1H-pyrazol-4-yl)-1H-indole-2-carboxylic acid (329)

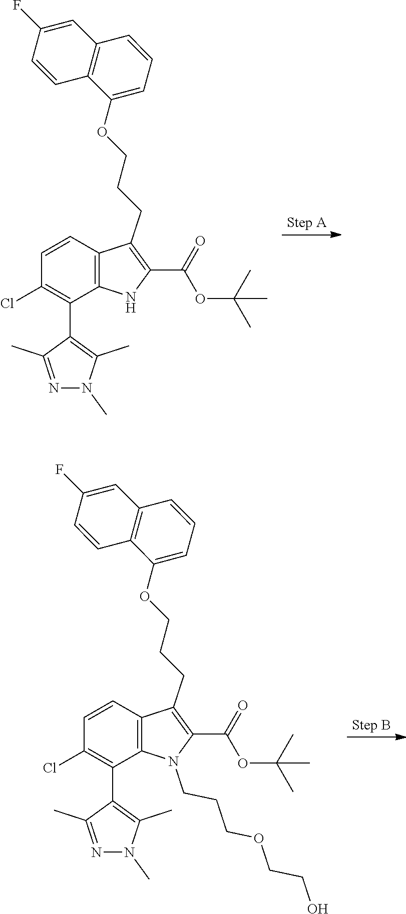

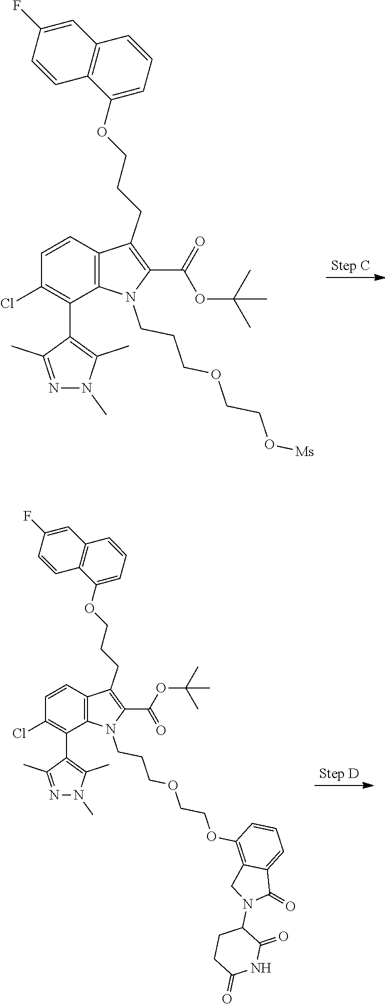 [SMILES: Cc1nn(C)c(C)c1-c1c(Cl)ccc2c(CCCOc3cccc4cc(F)ccc34)c(C(=O)OC(C)(C)C)n(CCCOCCOS(C)(=O)=O)c12 ; Cc1nn(C)c(C)c1-c1c(Cl)ccc2c(CCCOc3cccc4cc(F)ccc34)c(C(=O)OC(C)(C)C)n(CCCOCCOc3cccc4c3CN(C3CCC(=O)NC3=O)C4=O)c12]

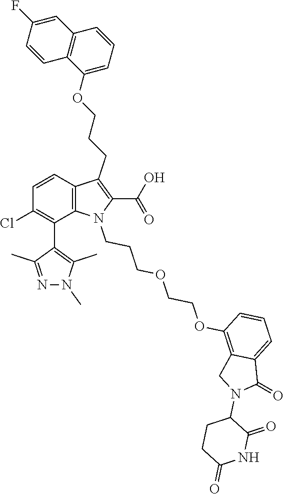 [SMILES: Cc1nn(C)c(C)c1-c1c(Cl)ccc2c(CCCOc3cccc4cc(F)ccc34)c(C(=O)O)n(CCCOCCOc3cccc4c3CN(C3CCC(=O)NC3=O)C4=O)c12]

### Step A

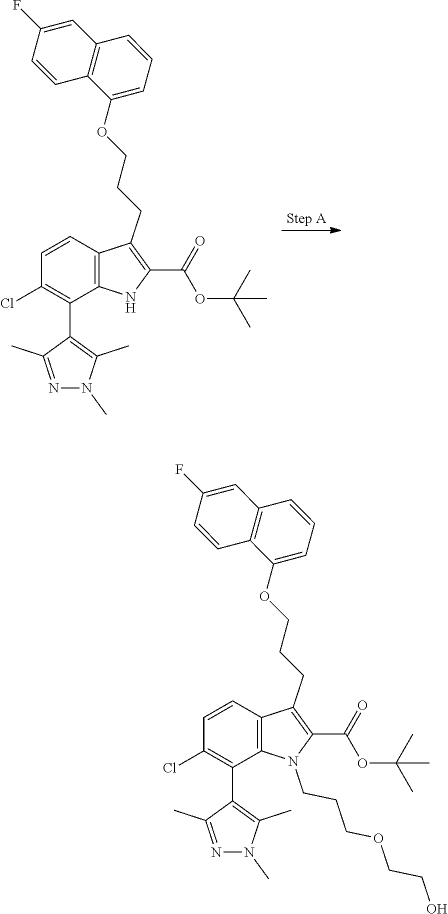

tert-Butyl 6-chloro-3-{3-[(6-fluoronaphthalen-1-yl)oxy]propyl}-7-(1,3,5-trimethyl-1H-pyrazol-4-yl)-1H-indole-2-carboxylate (50.0 mg, 0.089 mmol), 2-(3-chloropropoxy)ethanol (14.8 mg, 0.107 mmol), KI (14.8 mg, 0.089 mmol) and Cs₂CO₃ (86.9 mg, 0.267 mmol) were dissolved in dry DMF (2.0 mL) in an inert atmosphere and was stirred and stirred at 60° c. for weekend. After complete consumption of the starting material, the residues were dissolved in DCM and washed with water and brine. The organic layer was dried over MgSO₄, filtered and concentrated in vacuo to give a 53 mg of crude yellow oil tert-butyl 6-chloro-3-{3-[(6-fluoronaphthalen-1-yl)oxy]propyl}-1-[3-(2-hydroxyethoxy)propyl]-7-(1,3,5-trimethyl-1H-pyrazol-4-yl)-1H-indole-2-carboxylate, which was used to next step without further purification.

LCMS (ESI+): m/z 663.4 [M+H]⁺

### Step B

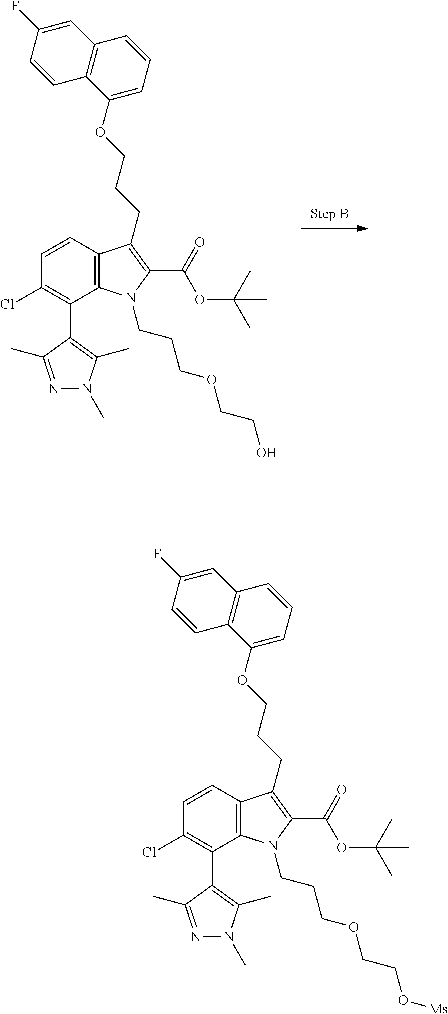

tert-Butyl 6-chloro-3-{3-[(6-fluoronaphthalen-1-yl)oxy]propyl}-1-[3-(2-hydroxyethoxy)propyl]-7-(1,3,5-trimethyl-1H-pyrazol-4-yl)-1H-indole-2-carboxylate (52.0 mg, 0.078 mmol) was dissolved in DCM (5.0 mL), Et₃N (0.033 mL, 0.235 mmol) and DMAP (1.0 mg, 0.008 mmol) were added to the mixture and reaction was cooled to 0° C. Then MsCl (0.012 mL, 0.157 mmol) was added dropwise and reaction mixture was let to stir at room temperature for 18 h. The crude was extracted with NaHCO₃, water and brine, dried over MgSO₄, filtered and concentrated in vacuo. tert-butyl 6-chloro-3-{3-[(6-fluoronaphthalen-1-yl)oxy]propyl}-1-{3-[2-(methanesulfonyloxy)ethoxy]propyl}-7-(1,3,5-trimethyl-1H-pyrazol-4-yl)-1H-indole-2-carboxylate (45 mg, crude) was a light yellow oil and was used to next step without further purification.

LCMS (ESI+): m/z 741.5 [M+H]⁺

### Step C

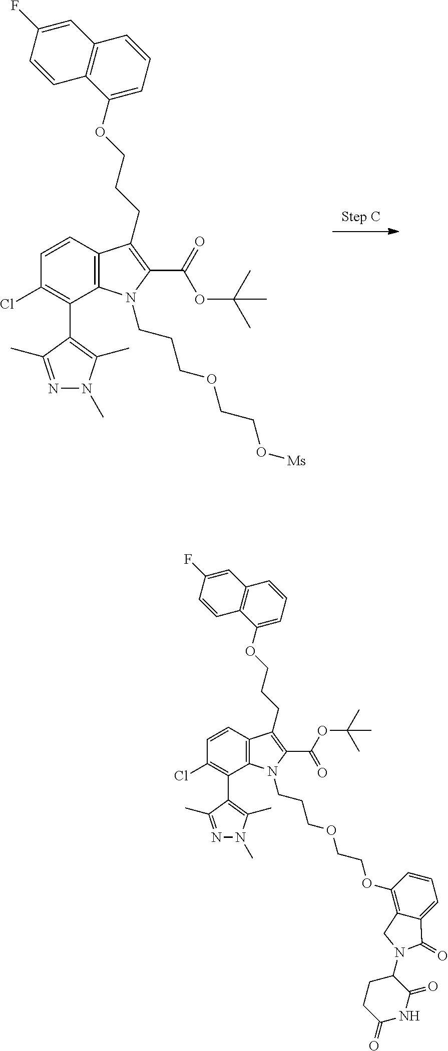 [SMILES: Cc1nn(C)c(C)c1-c1c(Cl)ccc2c(CCCOc3cccc4cc(F)ccc34)c(C(=O)OC(C)(C)C)n(CCCOCCOS(C)(=O)=O)c12 ; Cc1nn(C)c(C)c1-c1c(Cl)ccc2c(CCCOc3cccc4cc(F)ccc34)c(C(=O)OC(C)(C)C)n(CCCOCCOc3cccc4c3CN(C3CCC(=O)NC3=O)C4=O)c12]

To a solution of tert-butyl 6-chloro-3-{3-[(6-fluoronaphthalen-1-yl)oxy]propyl}-1-{3-[2-(methanesulfonyloxy)ethoxy]propyl}-7-(1,3,5-trimethyl-1H-pyrazol-4-yl)-1H-indole-2-carboxylate (45 mg crude) and 3-(4-hydroxy-1-oxo-2,3-dihydro-1H-isoindol-2-yl)piperidine-2,6-dione (23.7 mg, 0.091 mmol) in dry DMF (2.0 mL) in an inert atmosphere were added KHCO₃ (18.2 mg, 0.182 mmol) and KI (10.1 mg, 0.061 mmol). The mixture was stirred for 18 h at 60° C. Then, to the crude were added next portion of KHCO₃ (18.2 mg, 0.182 mmol) and KI (10.1 mg, 0.061 mmol) and let to stir for 48 h at 60° C. The mixture was suspended in DCM and washed with water and brine. The organic phase was dried over MgSO₄, filtered and concentrated in vacuo to obtain tert-butyl 6-chloro-1-[3-(2-{[2-(2,6-dioxopiperidin-3-yl)-1-oxo-2,3-dihydro-1H-isoindol-4-yl]oxy}ethoxy)propyl]-3-{3-[(6-fluoronaphthalen-1-yl)oxy]propyl}-7-(1,3,5-trimethyl-1H-pyrazol-4-yl)-1H-indole-2-carboxylate (38 mg, crude) as a yellow oil which was used to next step without purification.

LCMS (ESI+): m/z 906.6 [M+H]⁺

### Step D

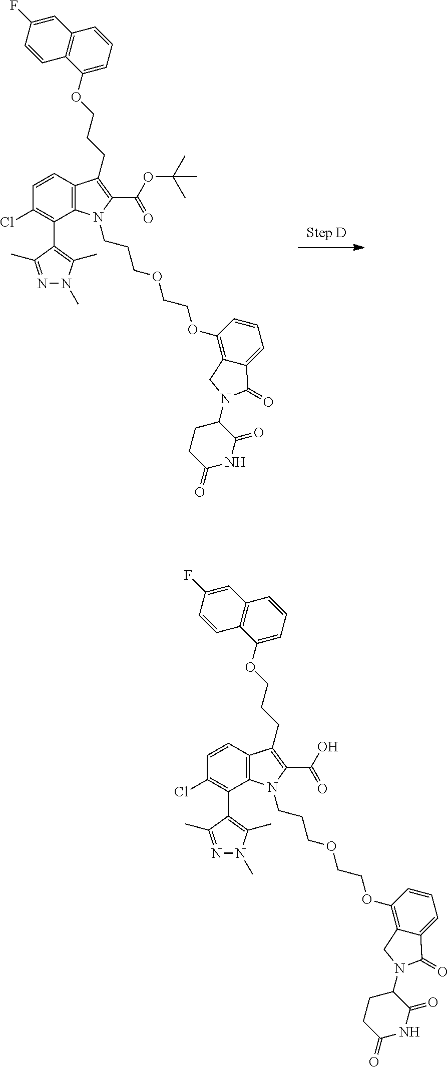 [SMILES: Cc1nn(C)c(C)c1-c1c(Cl)ccc2c(CCCOc3cccc4cc(F)ccc34)c(C(=O)OC(C)(C)C)n(CCCOCCOc3cccc4c3CN(C3CCC(=O)NC3=O)C4=O)c12 ; Cc1nn(C)c(C)c1-c1c(Cl)ccc2c(CCCOc3cccc4cc(F)ccc34)c(C(=O)O)n(CCCOCCOc3cccc4c3CN(C3CCC(=O)NC3=O)C4=O)c12]

To a solution of tert-butyl 6-chloro-1-[3-(2-{[2-(2,6-dioxopiperidin-3-yl)-1-oxo-2,3-dihydro-1H-isoindol-4-yl]oxy}ethoxy)propyl]-3-{3-[(6-fluoronaphthalen-1-yl)oxy]propyl}-7-(1,3,5-trimethyl-1H-pyrazol-4-yl)-1H-indole-2-carboxylate (38 mg, crude) was added TFA (1.0 mL, 13.059 mmol). The mixture was stirred for 18 h at RT. The crude was concentrated in vacuo and purified by preparative HPLC (C18, H₂O:MeCN+0.1% FA) and preparative TLC (7% MeOH in DCM) to obtain 6-chloro-1-[3-(2-{[2-(2,6-dioxopiperidin-3-yl)-1-oxo-2,3-dihydro-1H-isoindol-4-yl]oxy}ethoxy)propyl]-3-{3-[(6-fluoronaphthalen-1-yl)oxy]propyl}-7-(1,3,5-trimethyl-1H-pyrazol-4-yl)-1H-indole-2-carboxylic acid (6.3 mg, 0.007 mmol, 7.8% over four steps) as a white solid.

LCMS (ESI+): m/z 850.2 [M+H]⁺

¹H NMR (500 MHz, DMSO) δ 13.27 (s, 1H), 10.95 (s, 1H), 8.23 (dd, J=9.3, 5.9 Hz, 1H), 7.71 (d, J=8.5 Hz, 1H), 7.64 (dd, J=10.4, 2.7 Hz, 1H), 7.47 (td, J=7.8, 2.0 Hz, 1H), 7.44-7.41 (m, 2H), 7.36 (td, J=8.9, 2.7 Hz, 1H), 7.31 (dd, J=7.4, 1.7 Hz, 1H), 7.24-7.18 (m, 2H), 6.87 (dd, J=5.1, 3.7 Hz, 1H), 5.09 (ddd, J=13.3, 5.1, 2.2 Hz, 1H), 4.35 (dd, J=17.4, 4.7 Hz, 1H), 4.23-4.15 (m, 6H), 4.07-4.01 (m, 1H), 3.70 (d, J=11.6 Hz, 3H), 3.59-3.54 (m, 2H), 3.25 (t, J=7.6 Hz, 2H), 2.98-2.85 (m, 3H), 2.22-2.16 (m, 2H), 2.00-1.94 (m, 5H), 1.83 (d, J=4.0 Hz, 3H).

3 protons in aliphatic area overlaps with DMSO.

### Example 99. 6-Chloro-1-[3-(2-{[2-(2,6-dioxopiperidin-3-yl)-1-oxo-2,3-dihydro-1H-isoindol-4-yl]oxy}acetamido)propyl]-3-{3-[(6-fluoronaphthalen-1-yl)oxy]propyl}-7-(1,3,5-trimethyl-1H-pyrazol-4-yl)-1H-indole-2-carboxylic acid (330)

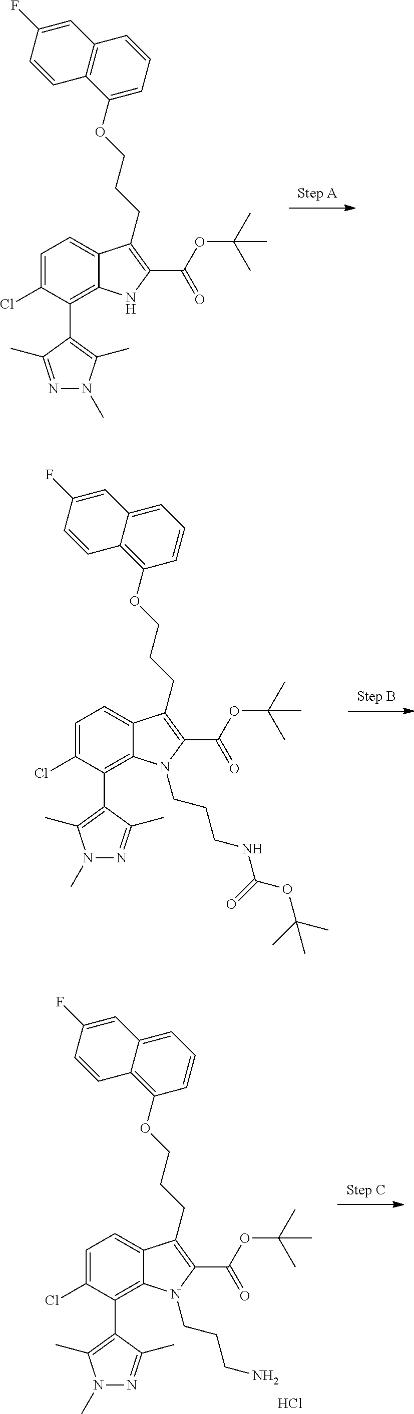

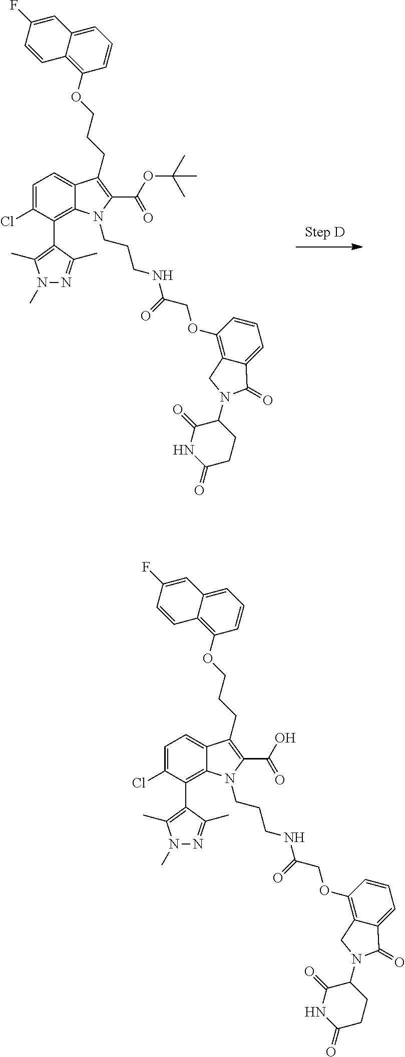 [SMILES: Cc1nn(C)c(C)c1-c1c(Cl)ccc2c(CCCOc3cccc4cc(F)ccc34)c(C(=O)OC(C)(C)C)n(CCCNC(=O)COc3cccc4c3CN(C3CCC(=O)NC3=O)C4=O)c12 ; Cc1nn(C)c(C)c1-c1c(Cl)ccc2c(CCCOc3cccc4cc(F)ccc34)c(C(=O)O)n(CCCNC(=O)COc3cccc4c3CN(C3CCC(=O)NC3=O)C4=O)c12]

### Step A

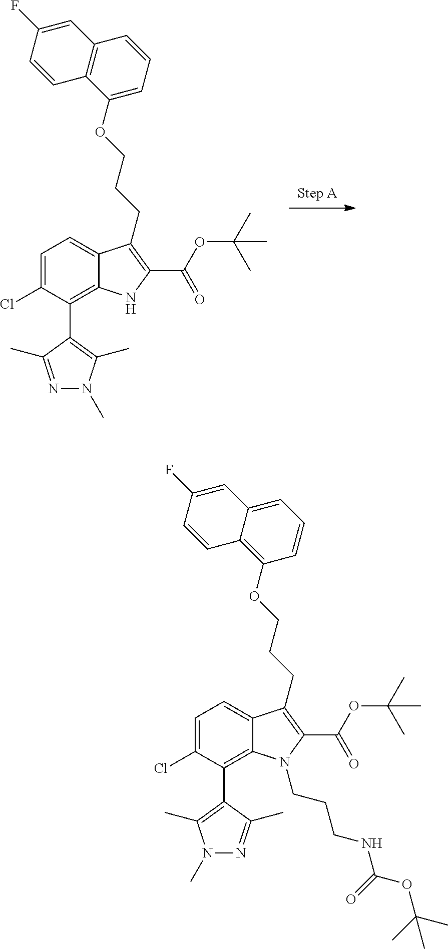 [SMILES: Cc1nn(C)c(C)c1-c1c(Cl)ccc2c(CCCOc3cccc4cc(F)ccc34)c(C(=O)OC(C)(C)C)[nH]c12 ; Cc1nn(C)c(C)c1-c1c(Cl)ccc2c(CCCOc3cccc4cc(F)ccc34)c(C(=O)OC(C)(C)C)n(CCCNC(=O)OC(C)(C)C)c12]

tert-Butyl 6-chloro-3-{3-[(6-fluoronaphthalen-1-yl)oxy]propyl}-7-(1,3,5-trimethyl-1H-pyrazol-4-yl)-1H-indole-2-carboxylate (50.0 mg, 0.089 mmol), tert-butyl N-(3-bromopropyl)carbamate (25.4 mg, 0.107 mmol), KI (14.8 mg, 0.089 mmol) and Cs₂CO₃ (86.9 mg, 0.267 mmol) were dissolved in dry DMF (2.0 mL) in an inert atmosphere and was stirred and stirred at 60° C. for 18 h. After complete consumption of the starting material (monitored by LCMS), the residues were dissolved in DCM and washed with brine. The organic layer was dried over MgSO₄, filtered and concentrated in vacuo to give a 93.2 mg of crude yellow oil of tert-butyl 1-(3-{[(tert-butoxy)carbonyl]amino}propyl)-6-chloro-3-{3-[(6-fluoronaphthalen-1-yl)oxy]propyl}-7-(1,3,5-trimethyl-1H-pyrazol-4-yl)-1H-indole-2-carboxylate which was used to next step without further purification.

LCMS (ESI+): m/z 720.4 [M+H]⁺

### Step B

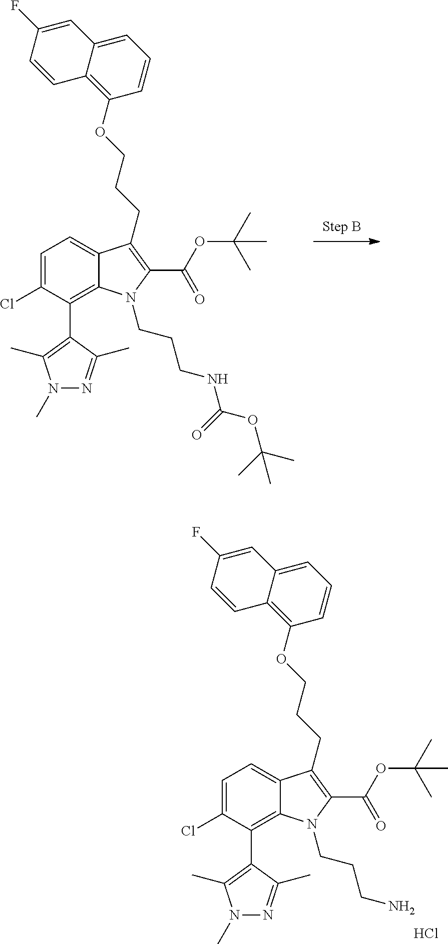

tert-Butyl 1-(3-{[(tert-butoxy)carbonyl]amino}propyl)-6-chloro-3-{3-[(6-fluoronaphthalen-1-yl)oxy]propyl}-7-(1,3,5-trimethyl-1H-pyrazol-4-yl)-1H-indole-2-carboxylate (93 mg, crude) was suspended in dry THE (4.7 mL), cooled to 0′ and to the mixture was added 4M HCl in dioxane (1.6 mL, 45.492 mmol). The reaction was stirred at room temperature for 18 h. The crude was concentrated in vacuo to get 83 mg crude of tert-butyl 1-(3-aminopropyl)-6-chloro-3-{3-[(6-fluoronaphthalen-1-yl)oxy]propyl}-7-(1,3,5-trimethyl-1H-pyrazol-4-yl)-1H-indole-2-carboxylate hydrochloride as a yellowish solid, which was used to next step without further purification.

LCMS (ESI+): m/z 619.1 [M+H]⁺

### Step C

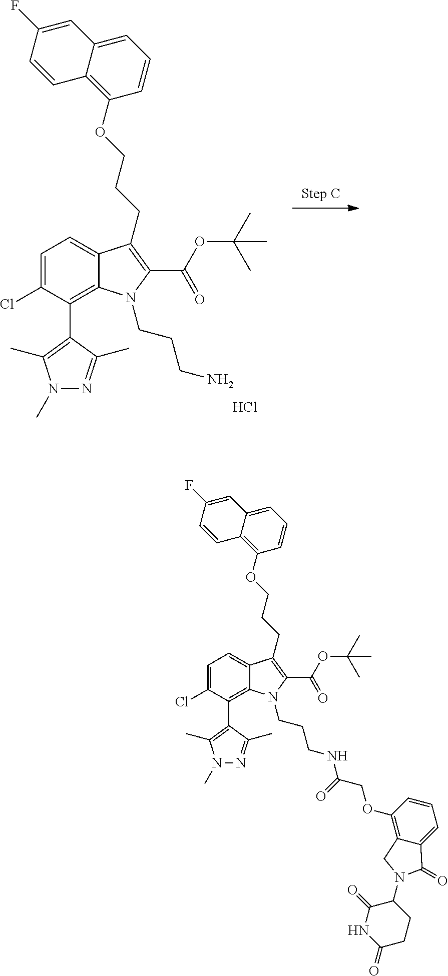 [SMILES: Cc1nn(C)c(C)c1-c1c(Cl)ccc2c(CCCOc3cccc4cc(F)ccc34)c(C(=O)OC(C)(C)C)n(CCCN)c12 ; Cl ; Cc1nn(C)c(C)c1-c1c(Cl)ccc2c(CCCOc3cccc4cc(F)ccc34)c(C(=O)OC(C)(C)C)n(CCCNC(=O)COc3cccc4c3CN(C3CCC(=O)NC3=O)C4=O)c12]

To a solution of tert-butyl 1-(3-aminopropyl)-6-chloro-3-{3-[(6-fluoronaphthalen-1-yl)oxy]propyl}-7-(1,3,5-trimethyl-1H-pyrazol-4-yl)-1H-indole-2-carboxylate hydrochloride (10.0 mg, crude), 2-{[2-(2,6-dioxopiperidin-3-yl)-1-oxo-2,3-dihydro-1H-isoindol-4-yl]oxy}acetic acid (5.8 mg, 0.018 mmol) and HATU (11.6 mg, 0.031 mmol) in dry DMF (1.3 mL) was added DIPEA (0.013 mL, 0.076 mmol). The mixture was stirred at room temperature for 30 min. The crude was diluted with DCM and washed with brine. The organic layer was dried over MgSO₄, filtered and concentrated in vacuo to get 30 mg crude with tert-butyl 6-chloro-1-[3-(2-{[2-(2,6-dioxopiperidin-3-yl)-1-oxo-2,3-dihydro-1H-isoindol-4-yl]oxy}acetamido)propyl]-3-{3-[(6-fluoronaphthalen-1-yl)oxy]propyl}-7-(1,3,5-trimethyl-1H-pyrazol-4-yl)-1H-indole-2-carboxylate as orange oil which was used in the next step without further purification.

LCMS (ESI+): m/z 919.1 [M+H]⁺

### Step D

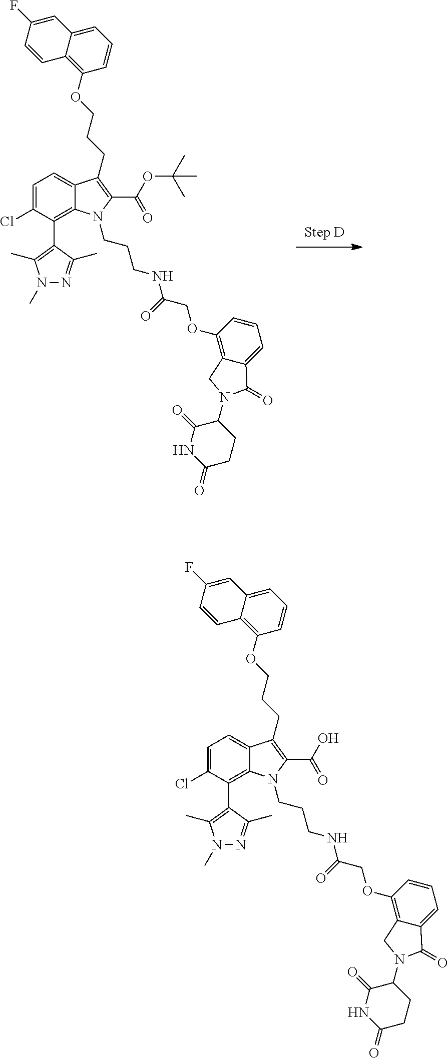 [SMILES: Cc1nn(C)c(C)c1-c1c(Cl)ccc2c(CCCOc3cccc4cc(F)ccc34)c(C(=O)OC(C)(C)C)n(CCCNC(=O)COc3cccc4c3CN(C3CCC(=O)NC3=O)C4=O)c12 ; Cc1nn(C)c(C)c1-c1c(Cl)ccc2c(CCCOc3cccc4cc(F)ccc34)c(C(=O)O)n(CCCNC(=O)COc3cccc4c3CN(C3CCC(=O)NC3=O)C4=O)c12]

To a solution of tert-butyl 6-chloro-1-[3-(2-{[2-(2,6-dioxopiperidin-3-yl)-1-oxo-2,3-dihydro-1H-isoindol-4-yl]oxy}acetamido)propyl]-3-{3-[(6-fluoronaphthalen-1-yl)oxy]propyl}-7-(1,3,5-trimethyl-1H-pyrazol-4-yl)-1H-indole-2-carboxylate (30 mg, crude) in DCM (1.0 mL) was added TFA (1.0 mL, 13.059 mmol). The mixture was stirred for 18 h at RT. The crude was concentrated in vacuo and purified by preparative HPLC (C18, H₂O:MeCN+0.1% FA) to obtain 6-chloro-1-[3-(2-{[2-(2,6-dioxopiperidin-3-yl)-1-oxo-2,3-dihydro-1H-isoindol-4-yl]oxy}acetamido)propyl]-3-{3-[(6-fluoronaphthalen-1-yl)oxy]propyl}-7-(1,3,5-trimethyl-1H-pyrazol-4-yl)-1H-indole-2-carboxylic acid (5.2 mg, 0.006 mmol, 60% over four steps) as a white solid.

LCMS (ESI+): m/z 863.2 [M+H]⁺

¹H NMR (500 MHz, DMSO, 353K) δ 12.77 (s, 1H), 10.64 (s, 1H), 8.26 (dd, J=9.2, 5.9 Hz, 1H), 7.85 (s, 1H), 7.65 (d, J=8.5 Hz, 1H), 7.58 (dd, J=10.4, 2.6 Hz, 1H), 7.46-7.39 (m, 3H), 7.37-7.31 (m, 2H), 7.16 (d, J=8.5 Hz, 1H), 7.13 (dt, J=8.2, 1.0 Hz, 1H), 6.87 (dd, J=5.9, 2.7 Hz, 1H), 5.06 (ddd, J=13.1, 5.3, 1.0 Hz, 1H), 4.59-4.51 (m, 3H), 4.42 (d, J=17.3 Hz, 1H), 4.25-4.17 (m, 3H), 4.05 (ddd, J=14.3, 9.1, 5.8 Hz, 1H), 3.76 (d, J=2.9 Hz, 3H), 3.25 (t, J=7.5 Hz, 2H), 2.88 (ddd, J=17.0, 13.4, 5.4 Hz, 1H), 2.79 (ddq, J=10.2, 6.7, 3.4 Hz, 1H), 2.71 (dq, J=13.1, 6.5 Hz, 1H), 2.65-2.59 (m, 1H), 2.25-2.19 (m, 2H), 2.07-2.02 (m, 1H), 1.97 (d, J=2.7 Hz, 3H), 1.87 (s, 3H), 1.44-1.32 (m, 2H).

1 proton in aliphatic area overlaps with DMSO.

### Example 100. 6-Chloro-1-(2-(4-((4-(2-(2,6-dioxopiperidin-3-yl)-6-fluoro-1,3-dioxoisoindolin-5-yl)piperazin-1-yl)methyl)piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (331)

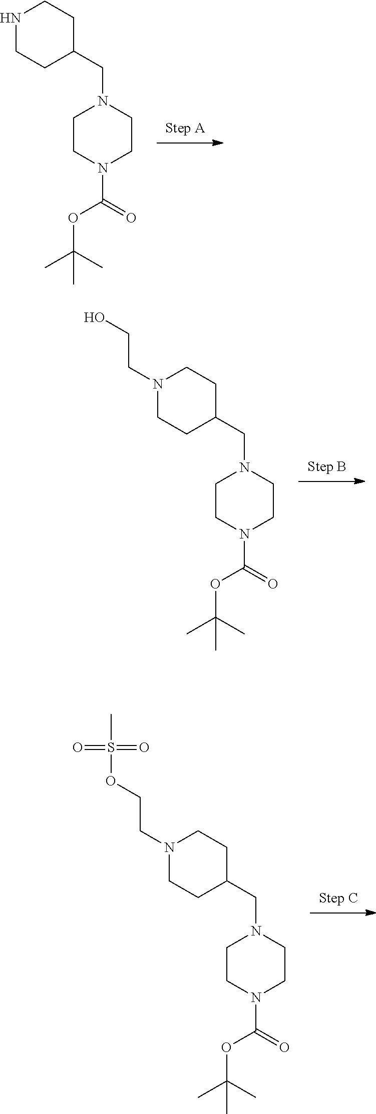

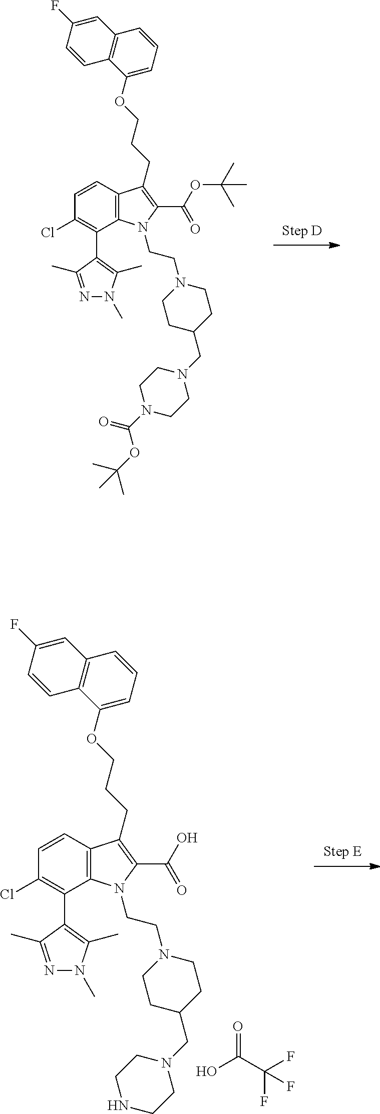

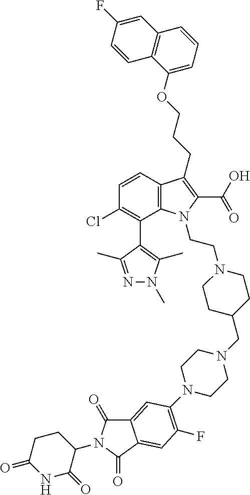 [SMILES: Cc1nn(C)c(C)c1-c1c(Cl)ccc2c(CCCOc3cccc4cc(F)ccc34)c(C(=O)O)n(CCN3CCC(CN4CCN(c5cc6c(cc5F)C(=O)N(C5CCC(=O)NC5=O)C6=O)CC4)CC3)c12]

### Step A

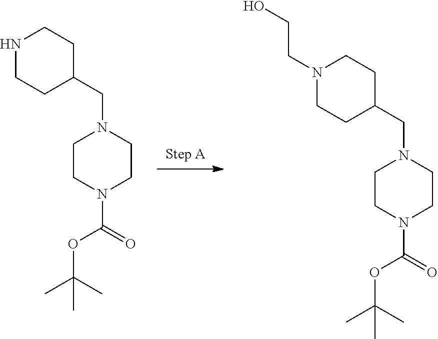 [SMILES: CC(C)(C)OC(=O)N1CCN(CC2CCNCC2)CC1 ; CC(C)(C)OC(=O)N1CCN(CC2CCN(CCO)CC2)CC1]

tert-Butyl 4-(piperidin-4-ylmethyl)piperazine-1-carboxylate (100.0 mg, 0.353 mmoL) and Cs₂CO₃ were dissolved in dry DMF (1.4 mL), stirred together at room temperature for 10 min and 2-bromoethanol (0.125 mL, 1.764 mmol) was added dropwise. Reaction was stirred in a closed flask under argon atmosphere for 2 h at 80° C. After that time, DMF was evaporated, resulting residue diluted with DCM and washed with brine and water. Organic phase was concentrated and dried under reduced pressure to give tert-butyl 4-((1-(2-hydroxyethyl)piperidin-4-yl)methyl)piperazine-1-carboxylate (95.4 mg, crude) which was used in the next step without further purification.

LCMS (ESI+): m/z 328.2 [M+H]⁺

### Step B

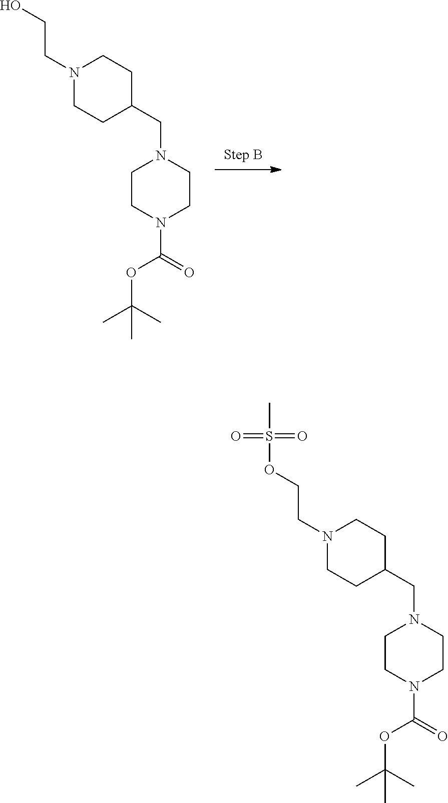 [SMILES: CC(C)(C)OC(=O)N1CCN(CC2CCN(CCO)CC2)CC1 ; CC(C)(C)OC(=O)N1CCN(CC2CCN(CCOS(C)(=O)=O)CC2)CC1]

tert-Butyl 4-((1-(2-hydroxyethyl)piperidin-4-yl)methyl)piperazine-1-carboxylate (95.4 mg, crude) was dissolved under argon atmosphere in dry DCM (2.9 mL) and Et₃N (0.061 mL, 0.437 mmol) along with DMAP (0.004 g, 0.029 mmol) were added. Reaction mixture was cooled down to 0° C. and MsCl (0.027 mL, 0.350 mmol) was added dropwise. Reaction was allowed to slowly reach room temperature and was stirred for 2 h. After that time, the solution was diluted with DCM and washed with brine and water. Organic layer was dried over anhydrous MgSO₄, filtered and dried under reduced pressure to give tert-butyl 4-((1-(2-((methylsulfonyl)oxy)ethyl)piperidin-4-yl)methyl)piperazine-1-carboxylate (89.8 mg, crude) as yellow oil. The resulting product was used in the next step without additional purification.

LCMS (ESI+): m/z 406.0 [M+H]⁺

### Step C

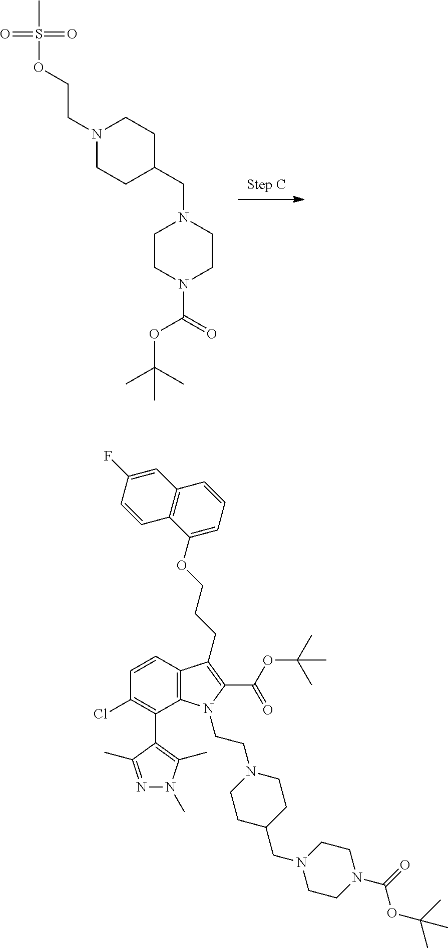 [SMILES: CC(C)(C)OC(=O)N1CCN(CC2CCN(CCOS(C)(=O)=O)CC2)CC1 ; Cc1nn(C)c(C)c1-c1c(Cl)ccc2c(CCCOc3cccc4cc(F)ccc34)c(C(=O)OC(C)(C)C)n(CCN3CCC(CN4CCN(C(=O)OC(C)(C)C)CC4)CC3)c12]

tert-Butyl 6-chloro-3-(3-((6-fluoronaphthalen-1-yl)oxy)propyl)-7-(1,3,5-trimethyl-1H-pyrazol-4-yl)-1H-indole-2-carboxylate (40.0 mg, 0.071 mmol) and Cs₂CO₃ (69.6 mg, 0.213 mmol) were dissolved in DMF (0.70 mL) under argon atmosphere and stirred for 5 min at room temperature. To it, crude tert-butyl 4-((1-(2-((methylsulfonyl)oxy)ethyl)piperidin-4-yl)methyl)piperazine-1-carboxylate (63.5 mg, 0.157 mmol) was added as a solution in DMF (0.70 mL) and the mixture stirred at room temperature under argon for 17 h. LCMS indicated complete conversion of the starting material. The mixture was diluted with DCM and washed with brine and water. Organic layer was dried over anhydrous Na₂SO₄, filtered and concentrated under reduced pressure to give tert-butyl 1-(2-(4-((4-(tert-butoxycarbonyl)piperazin-1-yl)methyl)piperidin-1-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate (90.5 mg, crude) as yellow oil. Reaction product was used in the next step without further purification.

LCMS (ESI+): m/z 871.2 [M+H]⁺

### Step D

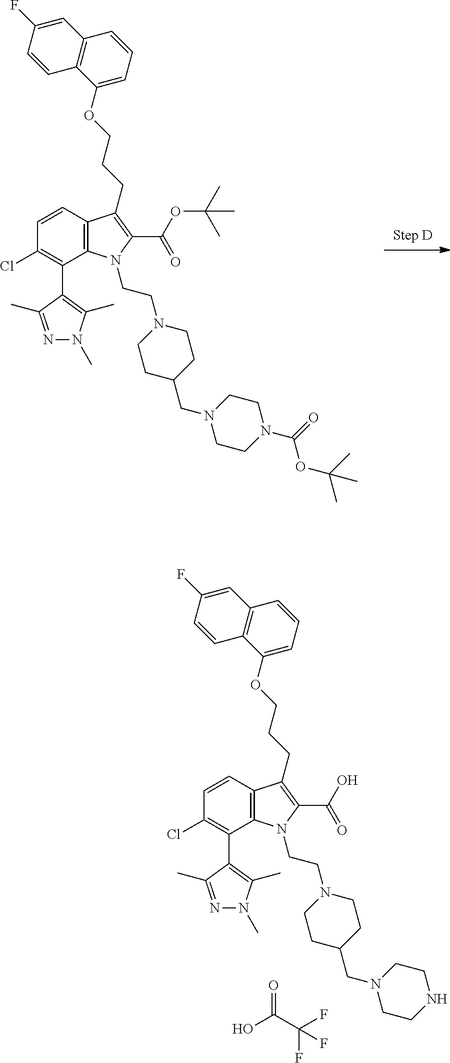

tert-Butyl 1-(2-(4-((4-(tert-butoxycarbonyl)piperazin-1-yl)methyl)piperidin-1-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate (44.8 mg, crude) was dissolved in dry DCM (0.500 mL) and TFA (0.500 mL, 6.529 mmol) was added. Reaction was stirred at room temperature under argon atmosphere for 16 h. Then, second portion of TFA (0.500 mL, 6.529 mmol) was added and the reaction was continued for 4 h. After complete conversion of the starting material, the solution was concentrated and dried under reduced pressure. 6-chloro-3-{3-[(6-fluoronaphthalen-1-yl)oxy]propyl}-1-(2-{4-[(piperazin-1-yl)methyl]piperidin-1-yl}ethyl)-7-(1,3,5-trimethyl-1H-pyrazol-4-yl)-1H-indole-2-carboxylic acid (112.5 mg, crude) as TFA salt was used directly in the next step without further purification.

LCMS (ESI+): m/z 715.1 [M+H]⁺

### Step E

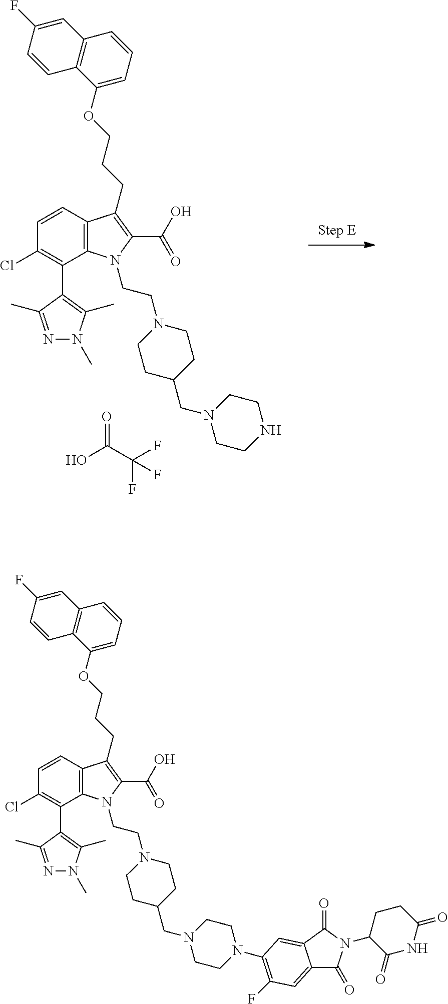 [SMILES: Cc1nn(C)c(C)c1-c1c(Cl)ccc2c(CCCOc3cccc4cc(F)ccc34)c(C(=O)O)n(CCN3CCC(CN4CCNCC4)CC3)c12 ; O=C(O)C(F)(F)F ; Cc1nn(C)c(C)c1-c1c(Cl)ccc2c(CCCOc3cccc4cc(F)ccc34)c(C(=O)O)n(CCN3CCC(CN4CCN(c5cc6c(cc5F)C(=O)N(C5CCC(=O)NC5=O)C6=O)CC4)CC3)c12]

6-Chloro-3-{3-[(6-fluoronaphthalen-1-yl)oxy]propyl}-1-(2-{4-[(piperazin-1-yl)methyl]piperidin-1-yl}ethyl)-7-(1,3,5-trimethyl-1H-pyrazol-4-yl)-1H-indole-2-carboxylic acid (TFA salt, 112.5 mg, crude) was dissolved in DMSO (1.6 mL) and DIPEA (0.274 mL, 1.573 mmol) along with 2-(2,6-dioxopiperidin-3-yl)-5,6-difluoroisoindoline-1,3-dione (46.3 mg, 0.157 mmol) was added. Reaction was stirred in a sealed vial at 90° C. for 15 Reaction mixture was cooled down and the crude product was purified with preparative HPLC (C18, H₂O:MeCN+0.1% FA) to 6-chloro-1-(2-(4-((4-(2-(2,6-dioxopiperidin-3-yl)-6-fluoro-1,3-dioxoisoindolin-5-yl)piperazin-1-yl)methyl)piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (11.4 mg, 0.012 mmol, 34% over 3 steps) as yellow solid

LCMS (ESI+): m/z 989.2 [M+H]⁺

¹H NMR (500 MHz, DMSO, 353 K) δ 10.77 (s, 1H), 8.25 (dd, J=9.3, 5.9 Hz, 1H), 7.67 (d, J=8.5 Hz, 1H), 7.63-7.54 (m, 2H), 7.45-7.40 (m, 2H), 7.39 (d, J=7.5 Hz, 1H), 7.35-7.29 (m, 1H), 7.21 (d, J=8.5 Hz, 1H), 6.87 (dd, J=5.2, 3.4 Hz, 1H), 5.05 (dd, J=12.5, 5.5 Hz, 1H), 4.31-4.13 (m, 4H), 3.77 (s, 3H), 3.26-3.21 (m, 6H), 2.88 (ddd, J=16.9, 13.5, 5.5 Hz, 1H), 2.66-2.52 (m, 4H), 2.49-2.45 (m, 4H), 2.35-2.28 (m, 2H), 2.28-2.16 (m, 4H), 2.13 (d, J=7.1 Hz, 2H), 2.10-2.04 (m, 1H), 2.02 (s, 3H), 1.89 (s, 3H), 1.69-1.57 (m, 2H), 1.57-1.40 (m, 1H), 1.22-1.09 (m, 2H).

### Example 101. 6-Chloro-1-(2-(4-((4-(2-(2,6-dioxopiperidin-3-yl)-1,3-dioxoisoindolin-4-yl)piperazin-1-yl)methyl)piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (332)

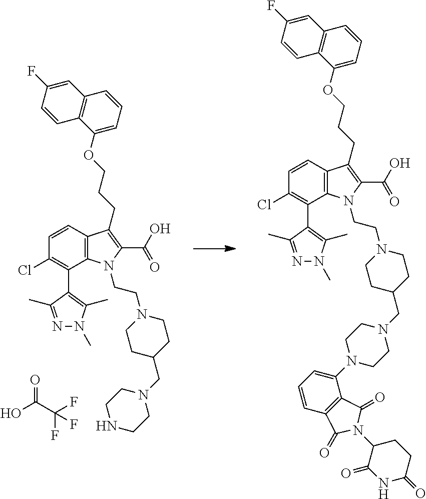 [SMILES: O=C(O)C(F)(F)F ; Cc1nn(C)c(C)c1-c1c(Cl)ccc2c(CCCOc3cccc4cc(F)ccc34)c(C(=O)O)n(CCN3CCC(CN4CCNCC4)CC3)c12 ; Cc1nn(C)c(C)c1-c1c(Cl)ccc2c(CCCOc3cccc4cc(F)ccc34)c(C(=O)O)n(CCN3CCC(CN4CCN(c5cccc6c5C(=O)N(C5CCC(=O)NC5=O)C6=O)CC4)CC3)c12]

6-Chloro-3-(3-((6-fluoronaphthalen-1-yl)oxy)propyl)-1-(2-(4-(piperazin-1-ylmethyl)piperidin-1-yl)ethyl)-7-(1,3,5-trimethyl-1H-pyrazol-4-yl)-1H-indole-2-carboxylic acid (TFA salt; 112.5 mg, crude) was dissolved in DMSO (1.6 mL) and DIPEA (0.274 mL, 1.573 mmol) along with 2-(2,6-dioxopiperidin-3-yl)-4-fluoro-2,3-dihydro-1H-isoindole-1,3-dione (43.4 mg, 0.157 mmol) was added. Reaction was stirred in a sealed vial at 90° C. for 15 h. Reaction mixture was cooled down and purified with preparative HPLC (C18, H₂O:MeCN+0.1% FA) to give 6-chloro-1-(2-(4-((4-(2-(2,6-dioxopiperidin-3-yl)-1,3-dioxoisoindolin-4-yl)piperazin-1-yl)methyl)piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (12.3 mg, 0.013 mmol, 37.1% over 3 steps—starting from 6-chloro-3-(3-((6-fluoronaphthalen-1-yl)oxy)propyl)-7-(1,3,5-trimethyl-1H-pyrazol-4-yl)-1H-indole-2-carboxylic acid).

LCMS (ESI+): m/z 971.2 [M+H]⁺

¹H NMR (500 MHz, DMSO) δ 11.06 (s, 1H), 8.25 (dd, J=9.3, 5.9 Hz, 1H), 7.73-7.58 (m, 3H), 7.47-7.40 (m, 2H), 7.40-7.32 (m, 2H), 7.27 (d, J=8.5 Hz, 1H), 7.20 (d, J=8.5 Hz, 1H), 6.92-6.83 (m, 1H), 5.08 (dd, J=12.8, 5.5 Hz, 1H), 4.32-4.07 (m, 4H), 3.76 (s, 3H), 3.22-3.14 (m, 6H), 2.87 (ddd, J=16.8, 13.9, 5.4 Hz, 1H), 2.61-2.52 (m, 3H), 2.48-2.35 (m, 9H), 2.23-2.15 (m, 2H), 2.07-1.95 (m, 6H), 1.88 (s, 3H), 1.67-1.57 (m, 2H), 1.57-1.49 (m, 1H), 1.18-1.07 (m, 2H).

### Example 102. 6-Chloro-1-[2-(4-{4-[2-(2,6-dioxopiperidin-3-yl)-6-fluoro-1,3-dioxo-2,3-dihydro-1H-isoindol-5-yl]piperazine-1-carbonyl}piperidin-1-yl)ethyl]-3-{3-[(6-fluoronaphthalen-1-yl)oxy]propyl}-7-(1,3,5-trimethyl-1H-pyrazol-4-yl)-1H-indole-2-carboxylic acid (333)

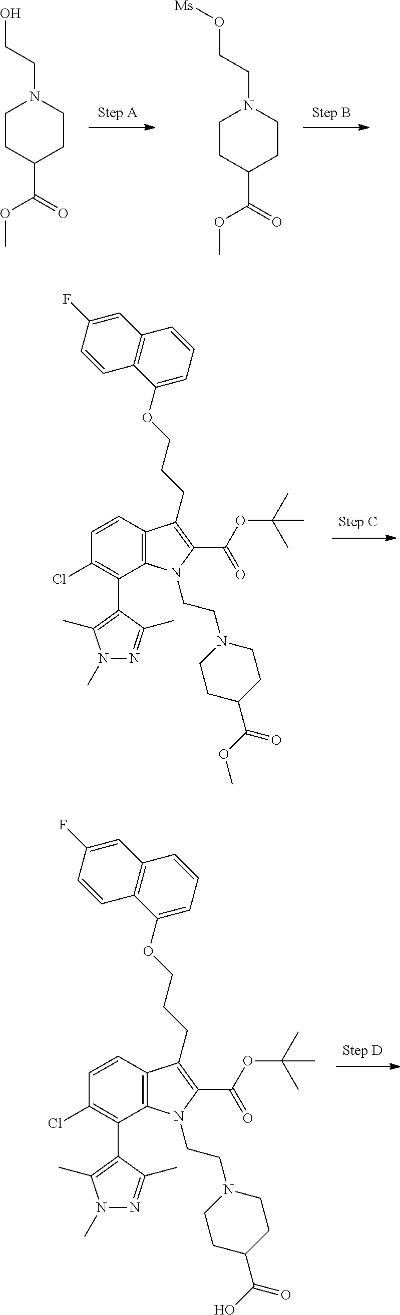

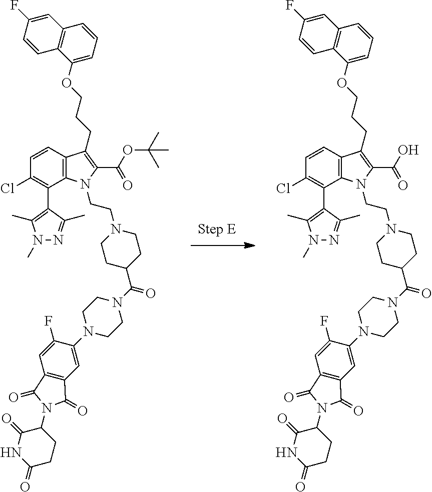 [SMILES: Cc1nn(C)c(C)c1-c1c(Cl)ccc2c(CCCOc3cccc4cc(F)ccc34)c(C(=O)OC(C)(C)C)n(CCN3CCC(C(=O)N4CCN(c5cc6c(cc5F)C(=O)N(C5CCC(=O)NC5=O)C6=O)CC4)CC3)c12 ; Cc1nn(C)c(C)c1-c1c(Cl)ccc2c(CCCOc3cccc4cc(F)ccc34)c(C(=O)O)n(CCN3CCC(C(=O)N4CCN(c5cc6c(cc5F)C(=O)N(C5CCC(=O)NC5=O)C6=O)CC4)CC3)c12]

### Step A

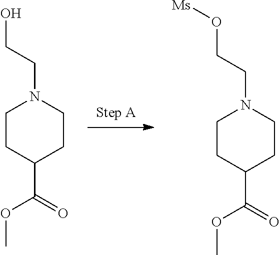 [SMILES: COC(=O)C1CCN(CCO)CC1 ; COC(=O)C1CCN(CCOS(C)(=O)=O)CC1]

Methyl 1-(2-hydroxyethyl)piperidine-4-carboxylate (130.7 mg, 0.698 mmol) was dissolved in DCM (5.0 mL), Et₃N (0.291 mL, 2.094 mmol) and DMAP (8.5 mg, 0.070 mmol) were added and reaction mixture cooled to 0° C. Then MsCl (0.081 mL, 1.047 mmol) was added dropwise and reaction mixture was let to stir at room temperature for 2 h. The crude was washed with brine, dried over MgSO₄, filtered and concentrated in vacuo. The isolated product methyl 1-[2-(methanesulfonyloxy)ethyl]piperidine-4-carboxylate (180 mg, crude) was an orange oil.

LCMS (ESI+): m/z 266.0 [M+H]⁺

### Step B

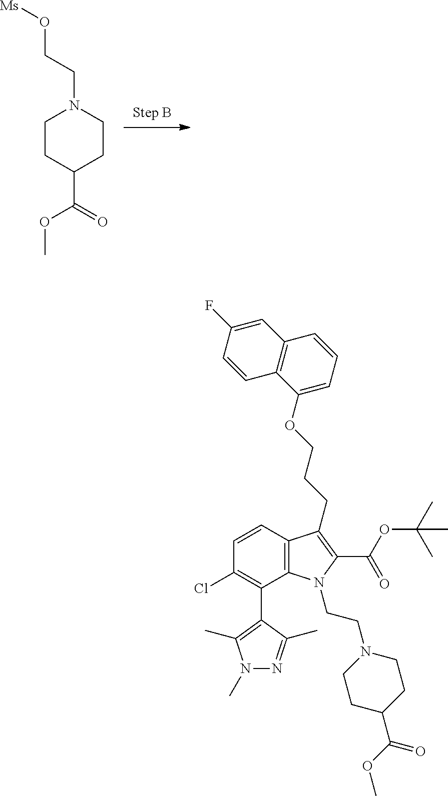 [SMILES: COC(=O)C1CCN(CCOS(C)(=O)=O)CC1 ; COC(=O)C1CCN(CCn2c(C(=O)OC(C)(C)C)c(CCCOc3cccc4cc(F)ccc34)c3ccc(Cl)c(-c4c(C)nn(C)c4C)c32)CC1]

To tert-butyl 6-chloro-3-{3-[(6-fluoronaphthalen-1-yl)oxy]propyl}-7-(1,3,5-trimethyl-1H-pyrazol-4-yl)-1H-indole-2-carboxylate (30.0 mg, 0.044 mmol) and Cs₂CO₃ (145.0 mg, 0.445 mmol) was added a solution of methyl 1-[2-(methanesulfonyloxy)ethyl]piperidine-4-carboxylate (60 mg, crude) in dry DMF (5.0 mL). The reaction was stirred for 36 h at 80° C. The crude was diluted in DCM and washed with brine. The organic layer was dried over MgSO₄, filtered and concentrated in vacuo to obtain tert-butyl 6-chloro-3-{3-[(6-fluoronaphthalen-1-yl)oxy]propyl}-1-{2-[4-(methoxycarbonyl)piperidin-1-yl]ethyl}-7-(1,3,5-trimethyl-1H-pyrazol-4-yl)-1H-indole-2-carboxylate (23 mg, crude) as an yellow oil, which was used to next step without further purification.

LCMS (ESI+): m/z 731.5 [M+H]⁺

### Step C

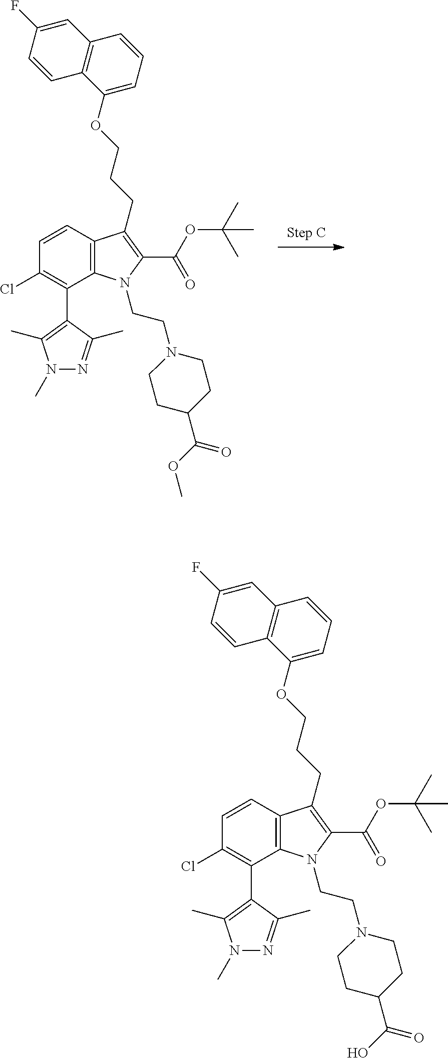 [SMILES: COC(=O)C1CCN(CCn2c(C(=O)OC(C)(C)C)c(CCCOc3cccc4cc(F)ccc34)c3ccc(Cl)c(-c4c(C)nn(C)c4C)c32)CC1 ; Cc1nn(C)c(C)c1-c1c(Cl)ccc2c(CCCOc3cccc4cc(F)ccc34)c(C(=O)OC(C)(C)C)n(CCN3CCC(C(=O)O)CC3)c12]

tert-Butyl 6-chloro-3-{3-[(6-fluoronaphthalen-1-yl)oxy]propyl}-1-{2-[4-(methoxycarbonyl)piperidin-1-yl]ethyl}-7-(1,3,5-trimethyl-1H-pyrazol-4-yl)-1H-indole-2-carboxylate (23 mg, crude) was dissolved in THE (1.0 mL) and aqueous 1M LiOH (0.315 mL). Reaction was stirred for 16 h at RT. After complete conversion of the starting material (monitored by LCMS) the solution pH was tuned to ˜6, resulting mixture was concentrated under reduced pressure and crude partitioned between 1-butanol and brine. Organic layer was dried over anhydrous MgSO₄, filtered and concentrated in vacuo to get 1-(2-{2-[(tert-butoxy)carbonyl]-6-chloro-3-{3-[(6-fluoronaphthalen-1-yl)oxy]propyl}-7-(1,3,5-trimethyl-1H-pyrazol-4-yl)-1H-indol-1-yl}ethyl)piperidine-4-carboxylic acid (21 mg, crude) as an yellow oil.

LCMS (ESI+): m/z 717.8 [M+H]⁺

### Step D

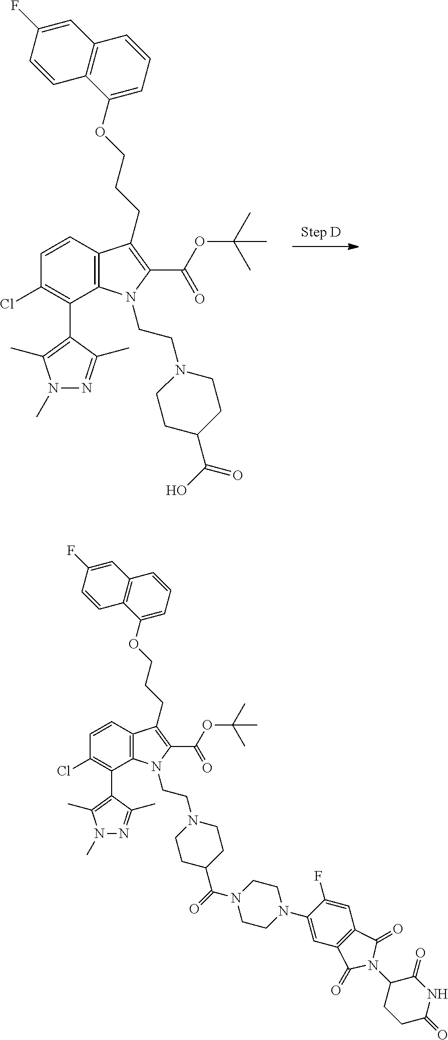 [SMILES: Cc1nn(C)c(C)c1-c1c(Cl)ccc2c(CCCOc3cccc4cc(F)ccc34)c(C(=O)OC(C)(C)C)n(CCN3CCC(C(=O)O)CC3)c12 ; Cc1nn(C)c(C)c1-c1c(Cl)ccc2c(CCCOc3cccc4cc(F)ccc34)c(C(=O)OC(C)(C)C)n(CCN3CCC(C(=O)N4CCN(c5cc6c(cc5F)C(=O)N(C5CCC(=O)NC5=O)C6=O)CC4)CC3)c12]

To a solution of 1-(2-{2-[(tert-butoxy)carbonyl]-6-chloro-3-{3-[(6-fluoronaphthalen-1-yl)oxy]propyl}-7-(1,3,5-trimethyl-1H-pyrazol-4-yl)-1H-indol-1-yl}ethyl)piperidine-4-carboxylic acid (21 mg, crude) and 2-(2,6-dioxopiperidin-3-yl)-5-fluoro-6-(piperazin-1-yl)-2,3-dihydro-1H-isoindole-1,3-dione (15.8 mg, 0.044 mmol) in dry DMF (2.0 mL) were added HATU (22.3 mg, 0.059 mmol) and DIPEA (0.025 mL, 0.146 mmol). The reaction was stirred at room temperature for 1 h. The crude was diluted in DCM and washed with brine. The organic layer was dried over MgSO₄, filtered and concentrated in vacuo to obtain tert-butyl 6-chloro-1-[2-(4-{4-[2-(2,6-dioxopiperidin-3-yl)-6-fluoro-1,3-dioxo-2,3-dihydro-1H-isoindol-5-yl]piperazine-1-carbonyl}piperidin-1-yl)ethyl]-3-[3-(naphthalen-1-yloxy)propyl]-7-(1,3,5-trimethyl-1H-pyrazol-4-yl)-1H-indole-2-carboxylate (68 mg, crude) as an yellow oil, which was used to next step without further purification.

LCMS (ESI+): m/z 1059.3 [M+H]⁺

### Step E

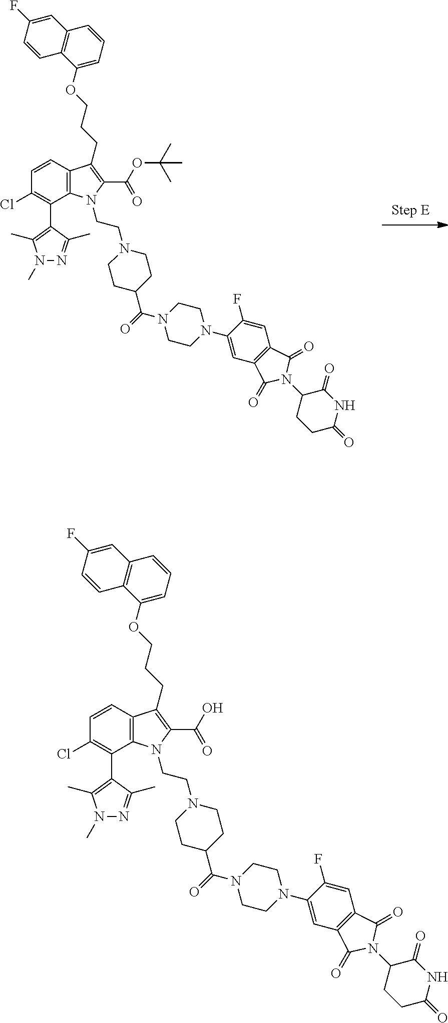 [SMILES: Cc1nn(C)c(C)c1-c1c(Cl)ccc2c(CCCOc3cccc4cc(F)ccc34)c(C(=O)OC(C)(C)C)n(CCN3CCC(C(=O)N4CCN(c5cc6c(cc5F)C(=O)N(C5CCC(=O)NC5=O)C6=O)CC4)CC3)c12 ; Cc1nn(C)c(C)c1-c1c(Cl)ccc2c(CCCOc3cccc4cc(F)ccc34)c(C(=O)O)n(CCN3CCC(C(=O)N4CCN(c5cc6c(cc5F)C(=O)N(C5CCC(=O)NC5=O)C6=O)CC4)CC3)c12]

To a solution of tert-butyl 6-chloro-1-[2-(4-{4-[2-(2,6-dioxopiperidin-3-yl)-6-fluoro-1,3-dioxo-2,3-dihydro-1H-isoindol-5-yl]piperazine-1-carbonyl}piperidin-1-yl)ethyl]-3-{3-[(6-fluoronaphthalen-1-yl)oxy]propyl}-7-(1,3,5-trimethyl-1H-pyrazol-4-yl)-1H-indole-2-carboxylate (68 mg, crude) in DCM (1 mL) was added TFA (1 mL). The reaction was stirred for 18 h at RT. The crude was concentrated in vacuo, dissolved in DMSO and purified by preparative HPLC (C18, H₂O:MeCN+0.1% FA) to obtain 6-chloro-1-[2-(4-{4-[2-(2,6-dioxopiperidin-3-yl)-6-fluoro-1,3-dioxo-2,3-dihydro-1H-isoindol-5-yl]piperazine-1-carbonyl}piperidin-1-yl)ethyl]-3-{3-[(6-fluoronaphthalen-1-yl)oxy]propyl}-7-(1,3,5-trimethyl-1H-pyrazol-4-yl)-1H-indole-2-carboxylic acid (9.0 mg, 0.009 mmol, 20.5% over four steps) as a light yellow solid.

LCMS (ESI+): m/z 1003.2 [M+H]⁺

¹H NMR (500 MHz, DMSO) δ 11.09 (s, 1H), 8.25 (dd, J=9.3, 5.9 Hz, 1H), 7.75 (d, J=11.2 Hz, 1H), 7.67 (d, J=8.6 Hz, 1H), 7.62 (dd, J=10.4, 2.6 Hz, 1H), 7.45-7.41 (m, 3H), 7.36 (td, J=8.9, 2.7 Hz, 1H), 7.20 (d, J=8.5 Hz, 1H), 6.87 (t, J=4.3 Hz, 1H), 5.10 (dd, J=12.9, 5.4 Hz, 1H), 4.29-4.21 (m, 1H), 4.20-4.13 (m, 3H), 3.76 (s, 3H), 3.61-3.54 (m, 4H), 3.23-3.13 (m, 8H), 2.88 (ddd, J=17.0, 13.8, 5.4 Hz, 1H), 2.65-2.51 (m, 3H), 2.35-2.25 (m, 4H), 2.19 (p, J=6.6 Hz, 2H), 2.06-2.00 (m, 4H), 1.88 (s, 3H), 1.63-1.52 (m, 4H).

### Example 103. 6-Chloro-1-(2-(4-((1-(2-(2,6-dioxopiperidin-3-yl)-6-fluoro-1,3-dioxoisoindolin-5-yl)azetidin-3-yl)methyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (334)

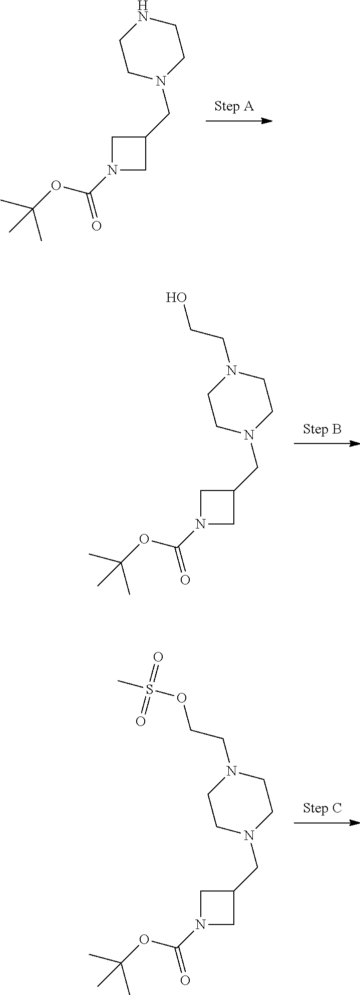 [SMILES: CC(C)(C)OC(=O)N1CC(CN2CCNCC2)C1 ; CC(C)(C)OC(=O)N1CC(CN2CCN(CCO)CC2)C1 ; CC(C)(C)OC(=O)N1CC(CN2CCN(CCOS(C)(=O)=O)CC2)C1]

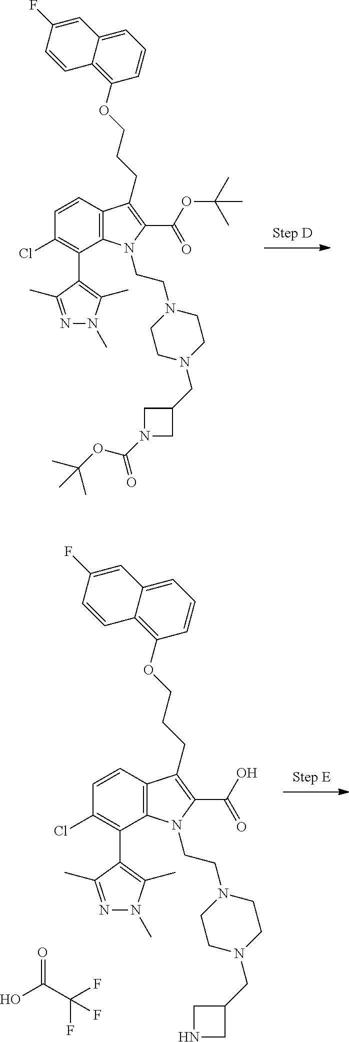

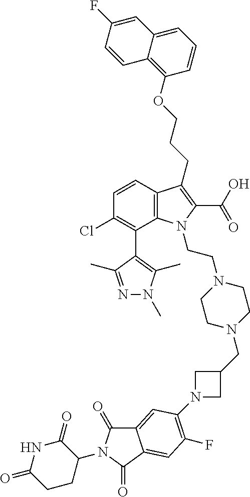 [SMILES: Cc1nn(C)c(C)c1-c1c(Cl)ccc2c(CCCOc3cccc4cc(F)ccc34)c(C(=O)O)n(CCN3CCN(CC4CN(c5cc6c(cc5F)C(=O)N(C5CCC(=O)NC5=O)C6=O)C4)CC3)c12]

### Step A

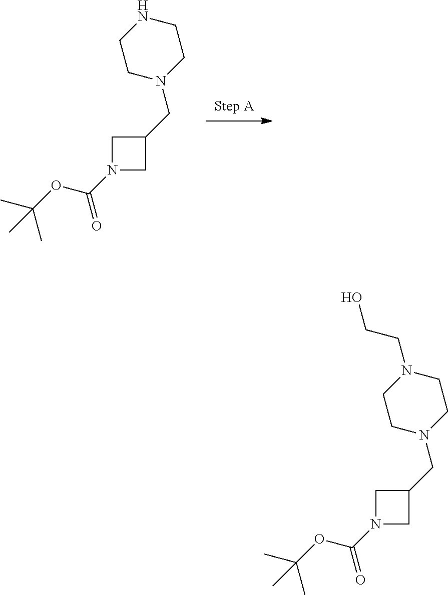 [SMILES: CC(C)(C)OC(=O)N1CC(CN2CCNCC2)C1 ; CC(C)(C)OC(=O)N1CC(CN2CCN(CCO)CC2)C1]

tert-Butyl 3-[(piperazin-1-yl)methyl]azetidine-1-carboxylate (66.9 mg, 0.262 mmol) was dissolved in dry DMF (2.6 mL) and Cs₂CO₃ (256.1 mg, 0.786 mmol) was added. The suspension was mixed for 15 min at room temperature and 2-bromoethanol (0.104 mL, 1.310 mmol) was added. The suspension was heated to 80° C. and the reaction was stirred for 2 h. The mixture was diluted with DCM and washed with brine and water. Organic layer was collected, dried over anhydrous MgSO₄, filtered and concentrated. The resulting residue was dried under reduced pressure to give tert-butyl 3-{[4-(2-hydroxyethyl)piperazin-1-yl]methyl}azetidine-1-carboxylate (64.5 mg, crude) as yellow oil that was directly used in the next step without further purification.

LCMS (ESI+): m/z 300.2 [M+H]⁺

### Step B

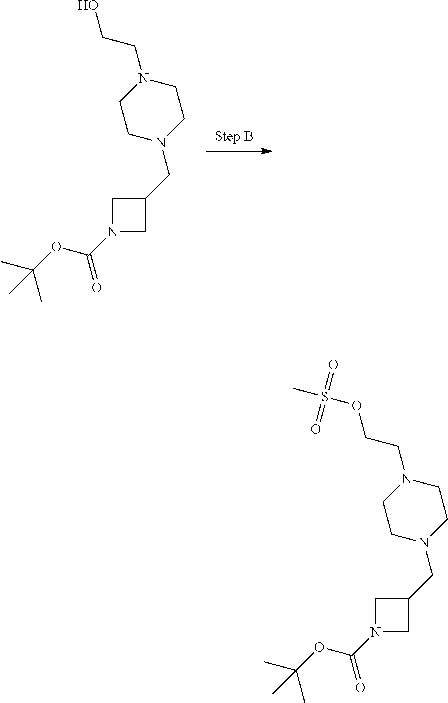 [SMILES: CC(C)(C)OC(=O)N1CC(CN2CCN(CCO)CC2)C1 ; CC(C)(C)OC(=O)N1CC(CN2CCN(CCOS(C)(=O)=O)CC2)C1]

tert-Butyl 3-{[4-(2-hydroxyethyl)piperazin-1-yl]methyl}azetidine-1-carboxylate (64.5 mg, crude) was dissolved in dry DCM (2.2 mL) under argon atmosphere and Et₃N (0.090 mL, 0.647 mmol) along with DMAP (2.6 mg, 0.022 mmol) were added. The solution was cooled down to 0° C. To it, MsCl (0.025 mL, 0.323 mmol) was added and the reaction was allowed to slowly reach ambient temperature. After 1 h, the solution was concentrated under reduced pressure, and the resulting residue dissolved in DCM. The organic layer was washed with brine and water, dried over anhydrous MgSO₄, filtered, concentrated and dried under reduced pressure to give 66.4 mg crude of tert-butyl 3-({4-[2-(methanesulfonyloxy)ethyl]piperazin-1-yl}methyl)azetidine-1-carboxylate as yellow oil which was used in the next step without further purification.

LCMS (ESI+): m/z 378.1 [M+H]⁺

### Step C

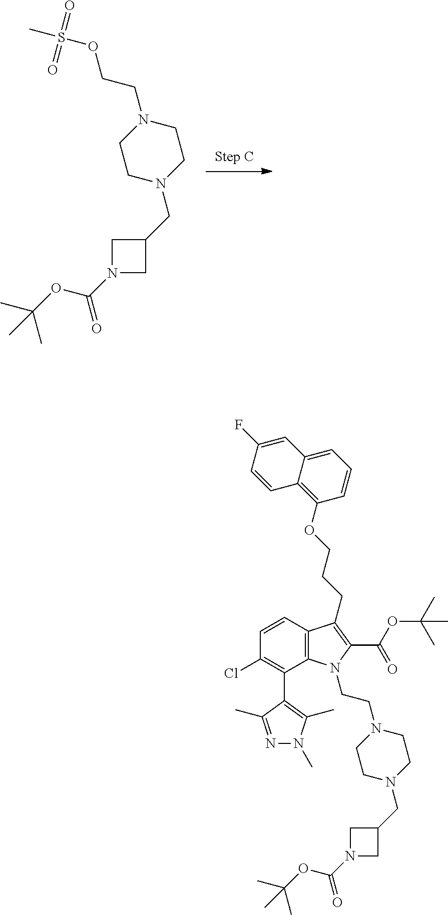 [SMILES: CC(C)(C)OC(=O)N1CC(CN2CCN(CCOS(C)(=O)=O)CC2)C1 ; Cc1nn(C)c(C)c1-c1c(Cl)ccc2c(CCCOc3cccc4cc(F)ccc34)c(C(=O)OC(C)(C)C)n(CCN3CCN(CC4CN(C(=O)OC(C)(C)C)C4)CC3)c12]

tert-Butyl 6-chloro-3-{3-[(6-fluoronaphthalen-1-yl)oxy]propyl}-7-(1,3,5-trimethyl-1H-pyrazol-4-yl)-1H-indole-2-carboxylate (40.0 mg, 0.071 mmol) was dissolved in dry DMF (2.8 mL) and Cs₂CO₃ (69.6 mg, 0.213 mmol) was added. The suspension was stirred for 15 min at room temperature and the crude tert-butyl 3-({4-[2-(methanesulfonyloxy)ethyl]piperazin-1-yl}methyl)azetidine-1-carboxylate (67.2 mg, 0.178 mmol) was added. Reaction was stirred for 16 h at room temperature in a sealed vial. After that, the mixture was heated to 80° C. and stirred at that temperature for 5 h. Additional portion of Cs₂CO₃ (46.4 mg, 0.142 mmol) was added and the reaction continued at 80° c. for 3 h. After LCMS indicated no further conversion, reaction mixture was cooled down to room temperature, diluted with DCM and washed with brine and water. Organic layer was collected, dried over anhydrous MgSO₄, filtered and concentrated. The resulting residue was dried under reduced pressure to give 81.3 mg crude of tert-butyl 1-{2-[4-({1-[(tert-butoxy)carbonyl]azetidin-3-yl}methyl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylate as yellow oil that was used in the next step without further purification.

LCMS (ESI+): m/z 843.5 [M+H]⁺

### Step D

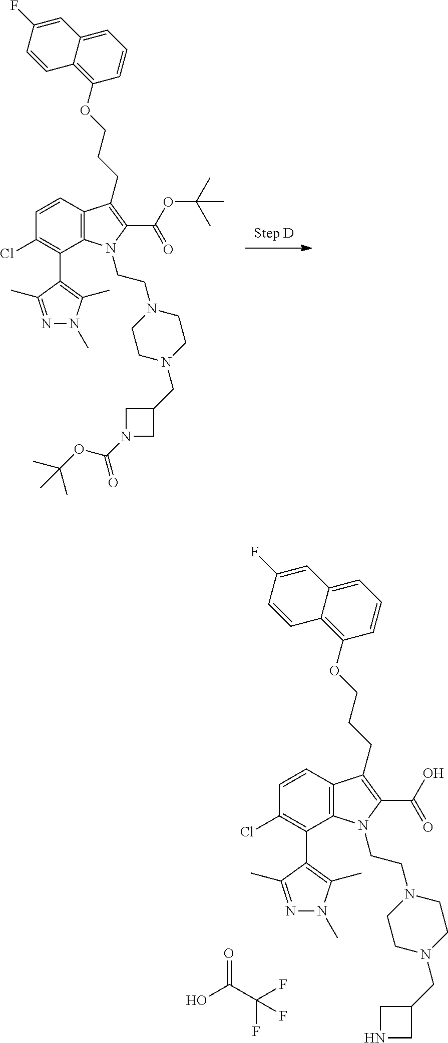 [SMILES: Cc1nn(C)c(C)c1-c1c(Cl)ccc2c(CCCOc3cccc4cc(F)ccc34)c(C(=O)OC(C)(C)C)n(CCN3CCN(CC4CN(C(=O)OC(C)(C)C)C4)CC3)c12 ; O=C(O)C(F)(F)F ; Cc1nn(C)c(C)c1-c1c(Cl)ccc2c(CCCOc3cccc4cc(F)ccc34)c(C(=O)O)n(CCN3CCN(CC4CNC4)CC3)c12]

tert-Butyl 1-{2-[4-({1-[(tert-butoxy)carbonyl]azetidin-3-yl}methyl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylate (81.3 mg, crude) was dissolved in dry DCM (0.740 mL) under argon atmosphere and TFA (0.740 mL, 9.663 mmol) was added. Reaction was stirred at room temperature under argon atmosphere for 16 h. After complete conversion of the starting material, the solution was concentrated and dried under reduced pressure to give 1-(2-{4-[(azetidin-3-yl)methyl]piperazin-1-yl}ethyl)-6-chloro-3-{3-[(6-fluoronaphthalen-1-yl)oxy]propyl}-7-(1,3,5-trimethyl-1H-pyrazol-4-yl)-1H-indole-2-carboxylic acid (TFA salt; 149.8 mg, crude) as dark oil.

LCMS (ESI+): m/z 687.3 [M+H]⁺

### Step E

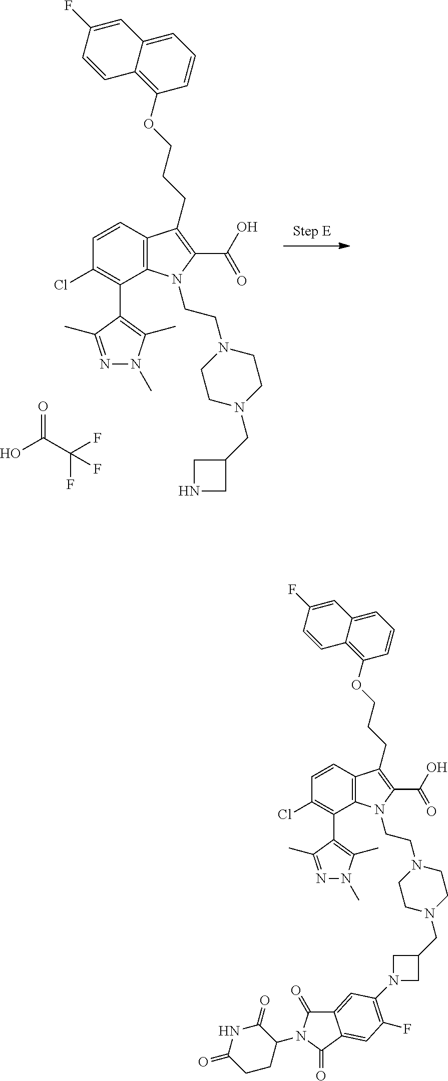 [SMILES: O=C(O)C(F)(F)F ; Cc1nn(C)c(C)c1-c1c(Cl)ccc2c(CCCOc3cccc4cc(F)ccc34)c(C(=O)O)n(CCN3CCN(CC4CNC4)CC3)c12 ; Cc1nn(C)c(C)c1-c1c(Cl)ccc2c(CCCOc3cccc4cc(F)ccc34)c(C(=O)O)n(CCN3CCN(CC4CN(c5cc6c(cc5F)C(=O)N(C5CCC(=O)NC5=O)C6=O)C4)CC3)c12]

1-(2-{4-[(Azetidin-3-yl)methyl]piperazin-1-yl}ethyl)-6-chloro-3-{3-[(6-fluoronaphthalen-1-yl)oxy]propyl}-7-(1,3,5-trimethyl-1H-pyrazol-4-yl)-1H-indole-2-carboxylic acid (TFA salt; 149.8 mg, crude) was dissolved in dry DMSO (2.0 mL) and 2-(2,6-dioxopiperidin-3-yl)-5,6-difluoro-2,3-dihydro-1H-isoindole-1,3-dione (60.5 mg, 0.206 mmol) was added, followed by DIPEA (0.326 mL, 1.869 mmol). Reaction was stirred at 90° C. for 16 h. After complete consumption of the starting material, the solution was cooled down to room temperature and purified with preparative HPLC (C18, H₂O:MeCN+0.1% FA). 6-chloro-1-(2-(4-((1-(2-(2,6-dioxopiperidin-3-yl)-6-fluoro-1,3-dioxoisoindolin-5-yl)azetidin-3-yl)methyl)piperazin-1-yl)ethyl)-3-(3-((6-fluoronaphthalen-1-yl)oxy)propy)-7-(1,3,5-trimethyl-1H-pyrazol-4-yl)-1H-indole-2-carboxylic acid (3.6 mg, 0.004 mmol, 5.6% over 3 steps) was obtained as yellow solid.

LCMS (ESI+): m/z 961.25 [M+H]⁺

¹H NMR (500 MHz, DMSO, 353 K) δ 10.75 (s, 1H), 8.25 (dd, J=9.2, 5.9 Hz, 1H), 7.69 (d, J=8.5 Hz, 1H), 7.57 (dd, J=10.3, 2.6 Hz, 1H), 7.46 (d, J=11.3 Hz, 1H), 7.42 (d, J=4.3 Hz, 2H), 7.32 (ddd, J=9.2, 8.6, 2.6 Hz, 1H), 7.21 (d, J=8.5 Hz, 1H), 6.87 (p, J=4.7 Hz, 1H), 6.83 (d, J=7.7 Hz, 1H), 5.01 (dd, J=12.5, 5.5 Hz, 1H), 4.32-4.10 (m, 6H), 3.81-3.76 (m, 2H), 3.76 (s, 3H), 3.29-3.23 (m, 2H), 2.91-2.81 (m, 2H), 2.61-2.55 (m, 1H), 2.55-2.51 (m, 3H), 2.34-2.28 (m, 4H), 2.28-2.21 (m, 2H), 2.20-2.07 (m, 6H), 2.07-2.02 (m, 1H), 2.02 (s, 3H), 1.89 (s, 3H).

### Example 104. 6-Chloro-1-(2-(4-(1-(2-(2,6-dioxopiperidin-3-yl)-6-fluoro-1,3-dioxoisoindolin-5-yl)azetidin-3-yl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (335)

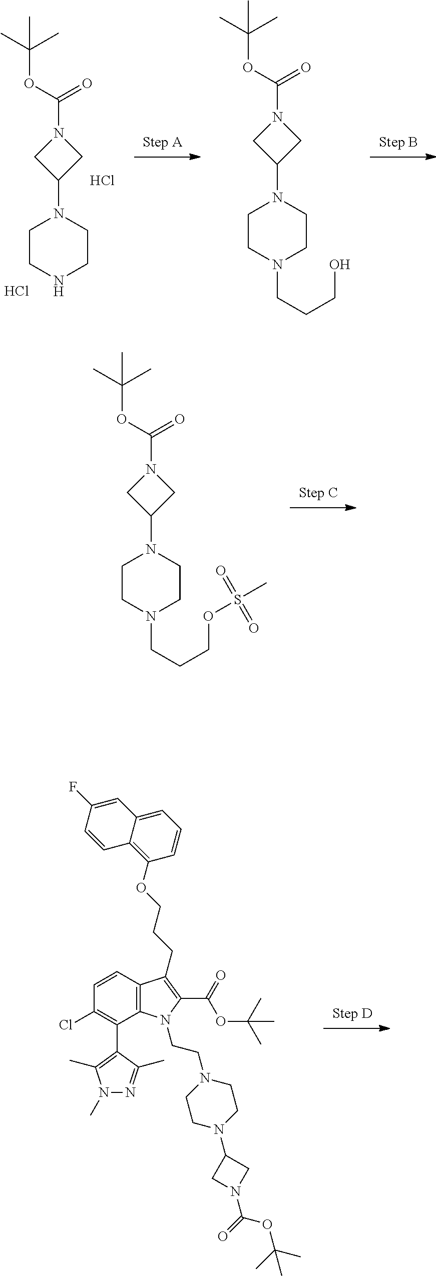 [SMILES: CC(C)(C)OC(=O)N1CC(N2CCNCC2)C1 ; Cl ; Cl ; CC(C)(C)OC(=O)N1CC(N2CCN(CCCO)CC2)C1 ; CC(C)(C)OC(=O)N1CC(N2CCN(CCCOS(C)(=O)=O)CC2)C1 ; Cc1nn(C)c(C)c1-c1c(Cl)ccc2c(CCCOc3cccc4cc(F)ccc34)c(C(=O)OC(C)(C)C)n(CCN3CCN(C4CN(C(=O)OC(C)(C)C)C4)CC3)c12]

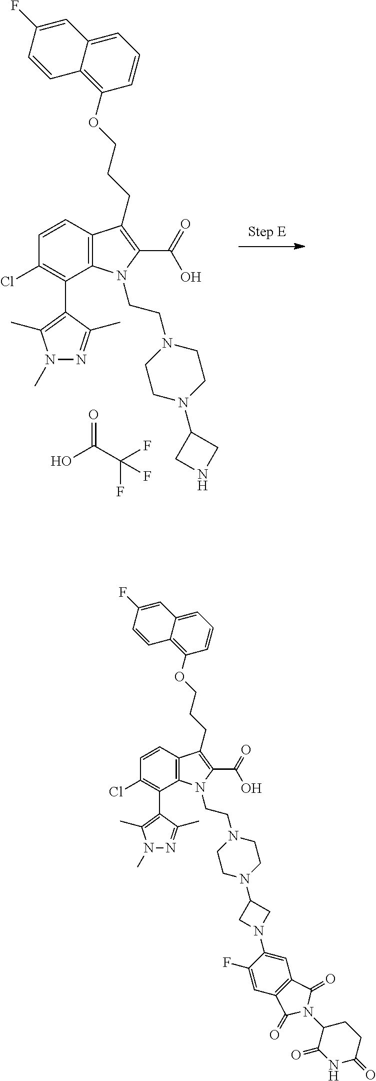 [SMILES: Cc1nn(C)c(C)c1-c1c(Cl)ccc2c(CCCOc3cccc4cc(F)ccc34)c(C(=O)O)n(CCN3CCN(C4CNC4)CC3)c12 ; O=C(O)C(F)(F)F ; Cc1nn(C)c(C)c1-c1c(Cl)ccc2c(CCCOc3cccc4cc(F)ccc34)c(C(=O)O)n(CCN3CCN(C4CN(c5cc6c(cc5F)C(=O)N(C5CCC(=O)NC5=O)C6=O)C4)CC3)c12]

### Step A

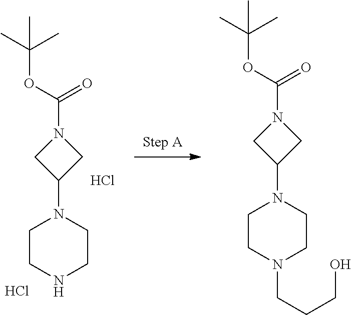 [SMILES: CC(C)(C)OC(=O)N1CC(N2CCNCC2)C1 ; Cl ; Cl ; CC(C)(C)OC(=O)N1CC(N2CCN(CCCO)CC2)C1]

tert-Butyl 3-(piperazin-1-yl)azetidine-1-carboxylate dihydrochloride (75.0 mg, 0.239 mmol) was dissolved in dry DMF (1.2 mL) under argon atmosphere and Cs₂CO₃ (311.0 mg, 0.955 mmol) was added. The suspension was stirred for 10 min and 2-bromoethan-1-ol (0.085 mL, 1.193 mmol) was added. Reaction was stirred for 3 h at 80° C. After complete consumption of the starting material the solution was diluted with DCM and then washed with brine and water. Organic layer was dried over anhydrous Na₂SO₄, filtered and dried under reduced pressure to give crude tert-butyl 3-[4-(2-hydroxyethyl)piperazin-1-yl]azetidine-1-carboxylate (73.8 mg) as yellow oil that was used in the next step without further purification.

LCMS (ESI+): m/z 286.2 [M+H]⁺

### Step B

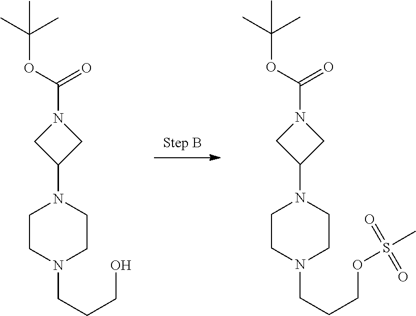 [SMILES: CC(C)(C)OC(=O)N1CC(N2CCN(CCCO)CC2)C1 ; CC(C)(C)OC(=O)N1CC(N2CCN(CCCOS(C)(=O)=O)CC2)C1]

Crude tert-butyl 3-[4-(2-hydroxyethyl)piperazin-1-yl]azetidine-1-carboxylate (73.8 mg) was dissolved in dry DCM (2.6 mL) under argon atmosphere and Et₃N (0.054 mL, 0.388 mmol) followed by pyridine (0.002 mL, 0.026 mmol) was added. The solution was cooled down to 0° C. and MsCl (0.024 mL, 0.311 mmol) was added. Reaction was allowed to reach room temperature and was continued for 2 h. After complete conversion of the starting material, the solution was diluted with DCM and washed with brine and water. Organic layer was dried over anhydrous MgSO₄, filtered and dried under reduced pressure to give crude tert-butyl 3-{4-[2-(methanesulfonyloxy)ethyl]piperazin-1-yl}azetidine-1-carboxylate (81.4 mg). Reaction product was obtained as yellow oil and was used in the following step without further purification.

### Step C

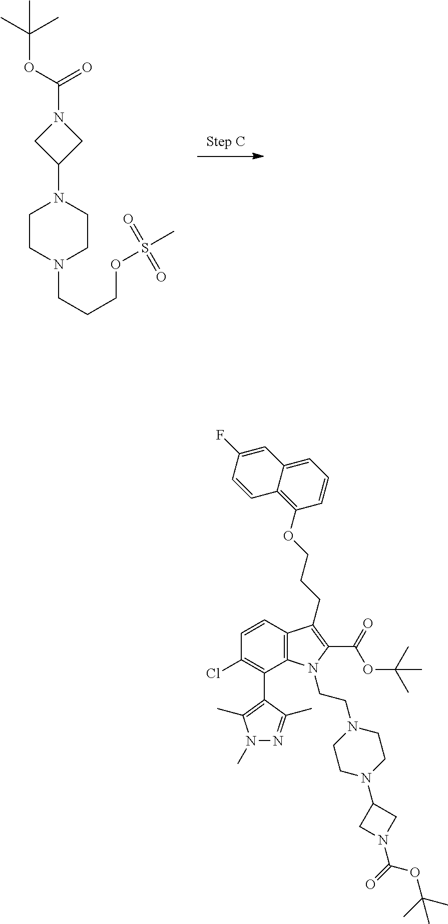 [SMILES: CC(C)(C)OC(=O)N1CC(N2CCN(CCCOS(C)(=O)=O)CC2)C1 ; Cc1nn(C)c(C)c1-c1c(Cl)ccc2c(CCCOc3cccc4cc(F)ccc34)c(C(=O)OC(C)(C)C)n(CCN3CCN(C4CN(C(=O)OC(C)(C)C)C4)CC3)c12]

tert-Butyl 6-chloro-3-{3-[(6-fluoronaphthalen-1-yl)oxy]propyl}-7-(1,3,5-trimethyl-1H-pyrazol-4-yl)-1H-indole-2-carboxylate (40.0 mg, 0.071 mmol) and Cs₂CO₃ (69.6 mg, 0.213 mmol) were dissolved in DMF (2.0 mL) under argon atmosphere and stirred for 15 min at room temperature. To it, crude tert-butyl 3-{4-[2-(methanesulfonyloxy)ethyl]piperazin-1-yl}azetidine-1-carboxylate (20.7 mg) was added as a solution in DMF (0.40 mL) and the mixture was stirred at room temperature under argon for 16 h. Then, additional portion of Cs₂CO₃ (23.2 mg, 0.071 mmol) was added and the mixture was heated to 60° C. over the next 40 h. Reaction was then cooled to ambient temperature, diluted with DCM and washed with brine and water. Organic layer was dried over anhydrous MgSO₄, filtered and concentrated under reduced pressure to give crude tert-butyl 1-[2-(4-{1-[(tert-butoxy)carbonyl]azetidin-3-yl}piperazin-1-yl)ethyl]-6-chloro-3-{3-[(6-fluoronaphthalen-1-yl)oxy]propyl}-7-(1,3,5-trimethyl-1H-pyrazol-4-yl)-1H-indole-2-carboxylate (66.0 mg) as yellow oil which was used in the next step without further purification.

LCMS (ESI+): m/z 829.1 [M+H]⁺

### Step D

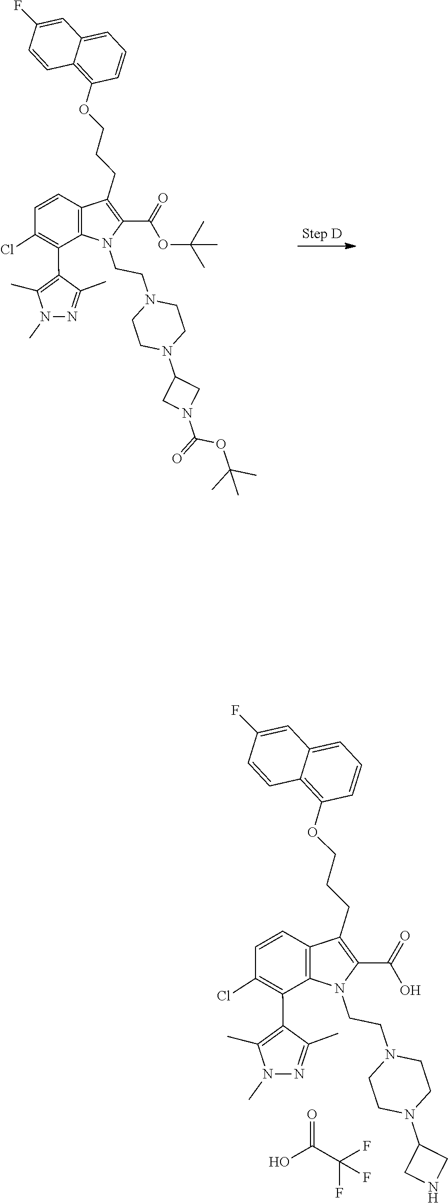 [SMILES: Cc1nn(C)c(C)c1-c1c(Cl)ccc2c(CCCOc3cccc4cc(F)ccc34)c(C(=O)OC(C)(C)C)n(CCN3CCN(C4CN(C(=O)OC(C)(C)C)C4)CC3)c12 ; Cc1nn(C)c(C)c1-c1c(Cl)ccc2c(CCCOc3cccc4cc(F)ccc34)c(C(=O)O)n(CCN3CCN(C4CNC4)CC3)c12 ; O=C(O)C(F)(F)F]

tert-Butyl 1-[2-(4-{1-[(tert-butoxy)carbonyl]azetidin-3-yl}piperazin-1-yl)ethyl]-6-chloro-3-{3-[(6-fluoronaphthalen-1-yl)oxy]propyl}-7-(1,3,5-trimethyl-1H-pyrazol-4-yl)-1H-indole-2-carboxylate (66.0 mg, crude) was dissolved in dry DCM (0.800 mL) under argon atmosphere and TFA (0.800 mL) was added. Reaction was stirred for 16 h at room temperature until complete consumption of the starting material. The solution was concentrated and dried under reduced pressure to give crude 1-{2-[4-(azetidin-3-yl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylic acid (TFA salt) as dark oil which was used in the next step without further purification.

LCMS (ESI+): m/z 673.7 [M+H]⁺

### Step E

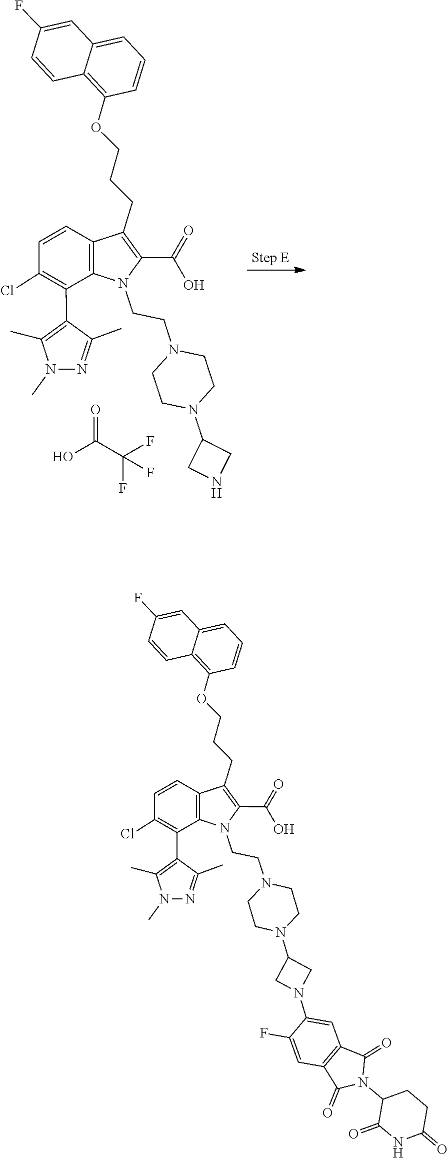 [SMILES: Cc1nn(C)c(C)c1-c1c(Cl)ccc2c(CCCOc3cccc4cc(F)ccc34)c(C(=O)O)n(CCN3CCN(C4CNC4)CC3)c12 ; O=C(O)C(F)(F)F ; Cc1nn(C)c(C)c1-c1c(Cl)ccc2c(CCCOc3cccc4cc(F)ccc34)c(C(=O)O)n(CCN3CCN(C4CN(c5cc6c(cc5F)C(=O)N(C5CCC(=O)NC5=O)C6=O)C4)CC3)c12]

Crude 1-{2-[4-(azetidin-3-yl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylic acid (TFA salt) was dissolved in DMSO (4.0 mL) and 2-(2,6-dioxopiperidin-3-yl)-5,6-difluoro-2,3-dihydro-1H-isoindole-1,3-dione (28.2 mg, 0.096 mmol), followed by DIPEA (0.139 mL, 0.800 mmol) were added. Reaction was conducted in a sealed vial for 16 h at 90° C. After complete conversion of the starting material, the solution purified with preparative HPLC (C18, H₂O:MeCN+0.1% FA) and preparative TLC (SiO₂, 10% MeOH in DCM) to give 6-chloro-1-(2-(4-(1-(2-(2,6-dioxopiperidin-3-yl)-6-fluoro-1,3-dioxoisoindolin-5-yl)azetidin-3-yl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (6.5 mg, 0.007 mmol, 9.9% over 3 steps) as pale yellow solid.

LCMS (ESI+): m/z 947.1 [M+H]⁺

¹H NMR (500 MHz, DMSO) δ 13.94 (s, 1H), 11.07 (s, 1H), 8.23 (dd, J=9.3, 5.9 Hz, 1H), 7.72-7.66 (m, 1H), 7.64 (dd, J=10.4, 2.7 Hz, 1H), 7.57 (d, J=11.1 Hz, 1H), 7.45-7.41 (m, 2H), 7.36 (td, J=8.9, 2.7 Hz, 1H), 7.19 (d, J=8.4 Hz, 1H), 6.93-6.80 (m, 2H), 5.06 (dd, J=12.9, 5.4 Hz, 1H), 4.37-4.25 (m, 1H), 4.19 (t, J=6.3 Hz, 2H), 4.17-4.08 (m, 3H), 3.95-3.85 (m, 2H), 3.74 (s, 3H), 3.24-3.15 (m, 3H), 2.87 (ddd, J=17.0, 13.9, 5.5 Hz, 1H), 2.62-2.53 (m, 2H), 2.31-1.96 (m, 16H), 1.86 (s, 3H).

### Example 105. 6-Chloro-1-[2-(4-{4-[2-(2,6-dioxopiperidin-3-yl)-6-fluoro-1,3-dioxo-2,3-dihydro-1H-isoindol-5-yl]piperazin-1-yl}piperidin-1-yl)ethyl]-3-{3-[(6-fluoronaphthalen-1-yl)oxy]propyl}-7-(1,3,5-trimethyl-1H-pyrazol-4-yl)-1H-indole-2-carboxylic acid (336)

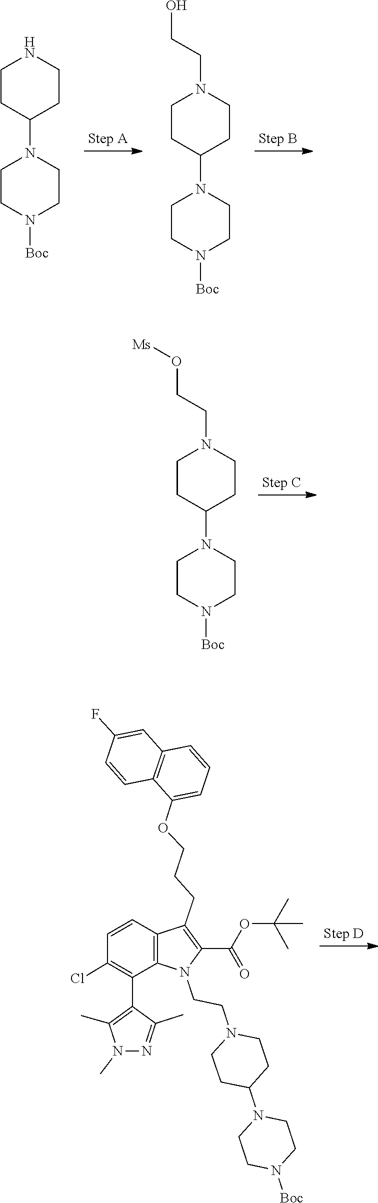 [SMILES: CC(C)(C)OC(=O)N1CCN(C2CCNCC2)CC1 ; CC(C)(C)OC(=O)N1CCN(C2CCN(CCO)CC2)CC1 ; CC(C)(C)OC(=O)N1CCN(C2CCN(CCOS(C)(=O)=O)CC2)CC1 ; Cc1nn(C)c(C)c1-c1c(Cl)ccc2c(CCCOc3cccc4cc(F)ccc34)c(C(=O)OC(C)(C)C)n(CCN3CCC(N4CCN(C(=O)OC(C)(C)C)CC4)CC3)c12]

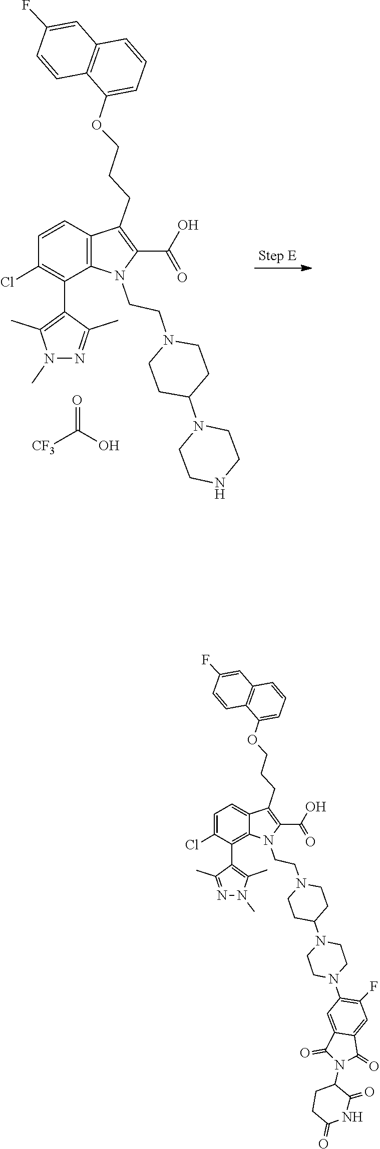 [SMILES: Cc1nn(C)c(C)c1-c1c(Cl)ccc2c(CCCOc3cccc4cc(F)ccc34)c(C(=O)O)n(CCN3CCC(N4CCNCC4)CC3)c12 ; O=C(O)C(F)(F)F ; Cc1nn(C)c(C)c1-c1c(Cl)ccc2c(CCCOc3cccc4cc(F)ccc34)c(C(=O)O)n(CCN3CCC(N4CCN(c5cc6c(cc5F)C(=O)N(C5CCC(=O)NC5=O)C6=O)CC4)CC3)c12]

### Step A

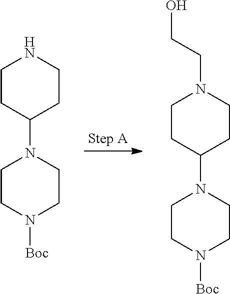 [SMILES: CC(C)(C)OC(=O)N1CCN(C2CCNCC2)CC1 ; CC(C)(C)OC(=O)N1CCN(C2CCN(CCO)CC2)CC1]

To a solution of tert-butyl 4-(piperidin-4-yl)piperazine-1-carboxylate (100.0 mg, 0.371 mmol) in DMF (2.0 mL) in an inert atmosphere were added Cs₂CO₃ (362.8 mg, 1.114 mmol) and 2-bromoethanol (0.132 mL, 1.856 mmol). The mixture was stirred at 60° C. for 18 h. The crude was diluted in DCM and washed with brine. The mixture with tert-butyl 4-[1-(2-hydroxyethyl)piperidin-4-yl]piperazine-1-carboxylate (91 mg, crude) was used to the next step without further purification.

### Step B

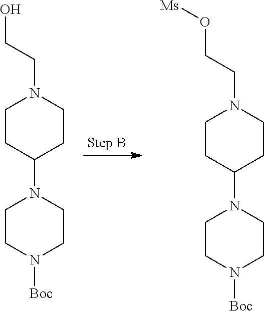 [SMILES: CC(C)(C)OC(=O)N1CCN(C2CCN(CCO)CC2)CC1 ; CC(C)(C)OC(=O)N1CCN(C2CCN(CCOS(C)(=O)=O)CC2)CC1]

tert-Butyl 4-[1-(2-hydroxyethyl)piperidin-4-yl]piperazine-1-carboxylate (91 mg, crude) was dissolved in DCM (5.0 mL), Et₃N (0.081 mL, 0.581 mmol) and DMAP (3.5 mg, 0.029 mmol) were added and reaction mixture cooled to 0° C. Then MsCl (0.027 mL, 0.348 mmol) was added dropwise and reaction mixture was let to stir at room temperature for 2 h. The crude was washed with brine, dried over MgSO₄, filtered and concentrated in vacuo. The isolated product tert-butyl 4-{1-[2-(methanesulfonyloxy)ethyl]piperidin-4-yl}piperazine-1-carboxylate (46.4 mg, crude) was an orange oil.

### Step C

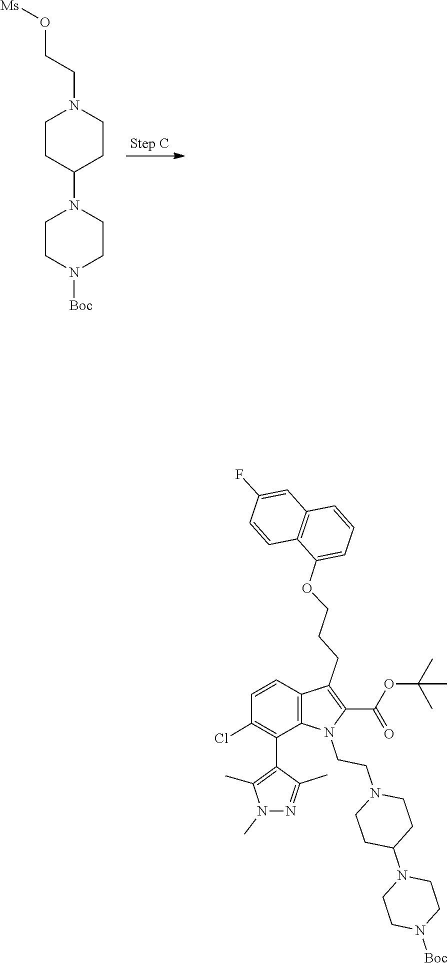 [SMILES: CC(C)(C)OC(=O)N1CCN(C2CCN(CCOS(C)(=O)=O)CC2)CC1 ; Cc1nn(C)c(C)c1-c1c(Cl)ccc2c(CCCOc3cccc4cc(F)ccc34)c(C(=O)OC(C)(C)C)n(CCN3CCC(N4CCN(C(=O)OC(C)(C)C)CC4)CC3)c12]

To tert-butyl 6-chloro-3-{3-[(6-fluoronaphthalen-1-yl)oxy]propyl}-7-(1,3,5-trimethyl-1H-pyrazol-4-yl)-1H-indole-2-carboxylate (30.0 mg, 0.053 mmol) and Cs₂CO₃ (86.9 mg, 0.267 mmol) was added tert-butyl 4-{1-[2-(methanesulfonyloxy)ethyl]piperidin-4-yl}piperazine-1-carboxylate (46 mg, crude) in dry DMF (2.0 mL). The mixture was stirred in an inert atmosphere for 18 h at 80° C. The crude was diluted in DCM and washed with brine. The organic layer was dried over MgSO₄, filtered and concentrated in vacuo to obtain tert-butyl 1-[2-(4-{4-[(tert-butoxy)carbonyl]piperazin-1-yl}piperidin-1-yl)ethyl]-6-chloro-3-{3-[(6-fluoronaphthalen-1-yl)oxy]propyl}-7-(1,3,5-trimethyl-1H-pyrazol-4-yl)-1H-indole-2-carboxylate (46.4 mg, crude) as a brown oil, which was used in the next step without further purification.

LCMS (ESI+): m/z 858.0 [M+H]⁺

### Step D

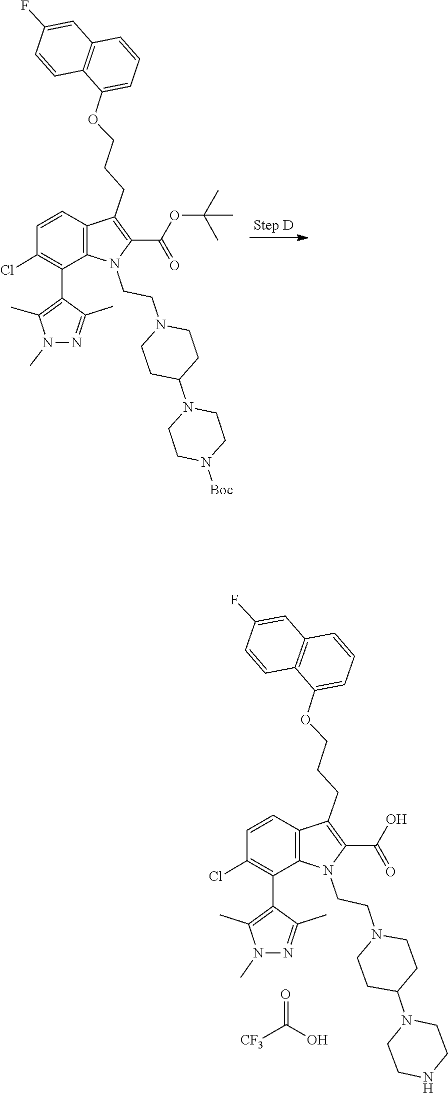 [SMILES: Cc1nn(C)c(C)c1-c1c(Cl)ccc2c(CCCOc3cccc4cc(F)ccc34)c(C(=O)OC(C)(C)C)n(CCN3CCC(N4CCN(C(=O)OC(C)(C)C)CC4)CC3)c12 ; Cc1nn(C)c(C)c1-c1c(Cl)ccc2c(CCCOc3cccc4cc(F)ccc34)c(C(=O)O)n(CCN3CCC(N4CCNCC4)CC3)c12 ; O=C(O)C(F)(F)F]

tert-Butyl 1-[2-(4-{4-[(tert-butoxy)carbonyl]piperazin-1-yl}piperidin-1-yl)ethyl]-6-chloro-3-{3-[(6-fluoronaphthalen-1-yl)oxy]propyl}-7-(1,3,5-trimethyl-1H-pyrazol-4-yl)-1H-indole-2-carboxylate (46.4 mg crude) in DCM (1.0 mL) was added TFA (1.0 mL, 13.059 mmol). The reaction was stirred at room temperature for 18 h. The crude was concentrated in vacuo to get 69 mg crude of 6-chloro-3-{3-[(6-fluoronaphthalen-1-yl)oxy]propyl}-1-{2-[4-(piperazin-1-yl)piperidin-1-yl]ethyl}-7-(1,3,5-trimethyl-1H-pyrazol-4-yl)-1H-indole-2-carboxylic acid trifluoroacetate as a brown oil, which was used to next step without further purification.

LCMS (ESI+): m/z 701.7 [M+H]⁺

### Step E

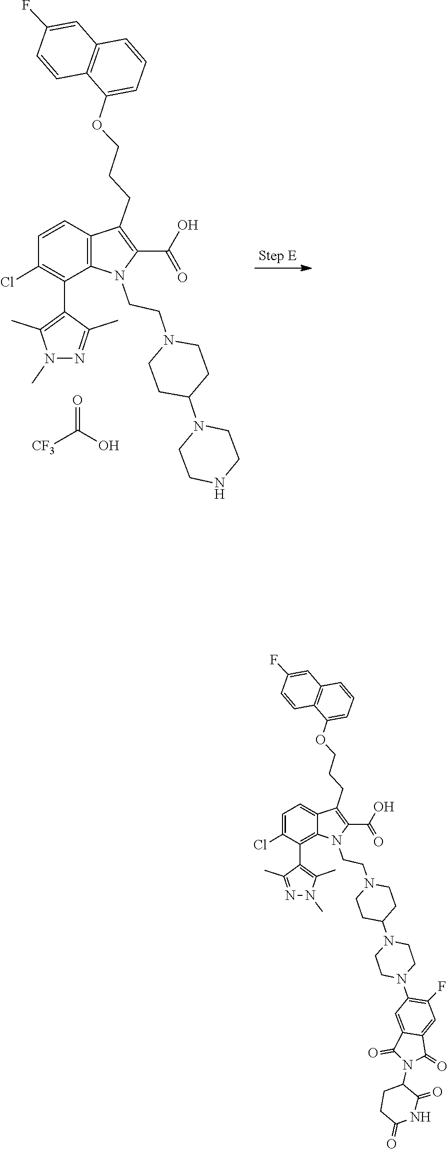 [SMILES: Cc1nn(C)c(C)c1-c1c(Cl)ccc2c(CCCOc3cccc4cc(F)ccc34)c(C(=O)O)n(CCN3CCC(N4CCNCC4)CC3)c12 ; O=C(O)C(F)(F)F ; Cc1nn(C)c(C)c1-c1c(Cl)ccc2c(CCCOc3cccc4cc(F)ccc34)c(C(=O)O)n(CCN3CCC(N4CCN(c5cc6c(cc5F)C(=O)N(C5CCC(=O)NC5=O)C6=O)CC4)CC3)c12]

To a solution of 2-(2,6-dioxopiperidin-3-yl)-5,6-difluoro-2,3-dihydro-1H-isoindole-1,3-dione (29.9 mg, 0.102 mmol) and 6-chloro-3-{3-[(6-fluoronaphthalen-1-yl)oxy]propyl}-1-{2-[4-(piperazin-1-yl)piperidin-1-yl]ethyl}-7-(1,3,5-trimethyl-1H-pyrazol-4-yl)-1H-indole-2-carboxylic acid trifluoroacetate (69 mg crude) in DMSO (2.0 mL) was added DIPEA (0.295 mL, 1.693 mmol) and the mixture was stirred at 90° C. for 18 h. The crude was purified by preparative HPLC (C18, H₂O:MeCN+0.1% FA) to obtain 6-chloro-1-[2-(4-{4-[2-(2,6-dioxopiperidin-3-yl)-6-fluoro-1,3-dioxo-2,3-dihydro-1H-isoindol-5-yl]piperazin-1-yl}piperidin-1-yl)ethyl]-3-{3-[(6-fluoronaphthalen-1-yl)oxy]propyl}-7-(1,3,5-trimethyl-1H-pyrazol-4-yl)-1H-indole-2-carboxylic acid (18.7 mg, 0.019 mmol, 35.8% overthree steps) as a yellowish solid.

LCMS (ESI+): m/z 975.20 [M+H]⁺

¹H NMR (500 MHz, DMSO, 353 K) δ 10.77 (s, 1H), 8.25 (dd, J=9.3, 5.9 Hz, 1H), 7.68 (d, J=8.5 Hz, 1H), 7.61-7.55 (m, 2H), 7.44-7.40 (m, 2H), 7.37 (d, J=7.5 Hz, 1H), 7.31 (ddd, J=9.3, 8.6, 2.6 Hz, 1H), 7.21 (d, J=8.5 Hz, 1H), 6.87 (m, 1H), 5.05 (dd, J=12.5, 5.5 Hz, 1H), 4.28-4.22 (m, 3H), 4.19-4.13 (m, 1H), 3.77 (s, 3H), 3.27-3.22 (m, 2H), 3.22-3.19 (m, 4H), 2.88 (ddd, J=16.7, 13.4, 5.3 Hz, 1H), 2.66-2.51 (m, 8H), 2.28-2.17 (m, 5H), 2.12-2.04 (m, 3H), 2.02 (s, 3H), 1.89 (s, 3H), 1.69-1.61 (m, 2H), 1.48-1.38 (m, 2H).

### Example 106. 6-Chloro-1-(2-(3-(4-(2-(2,6-dioxopiperidin-3-yl)-6-fluoro-1,3-dioxoisoindolin-5-yl)piperazin-1-yl)azetidin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (337)

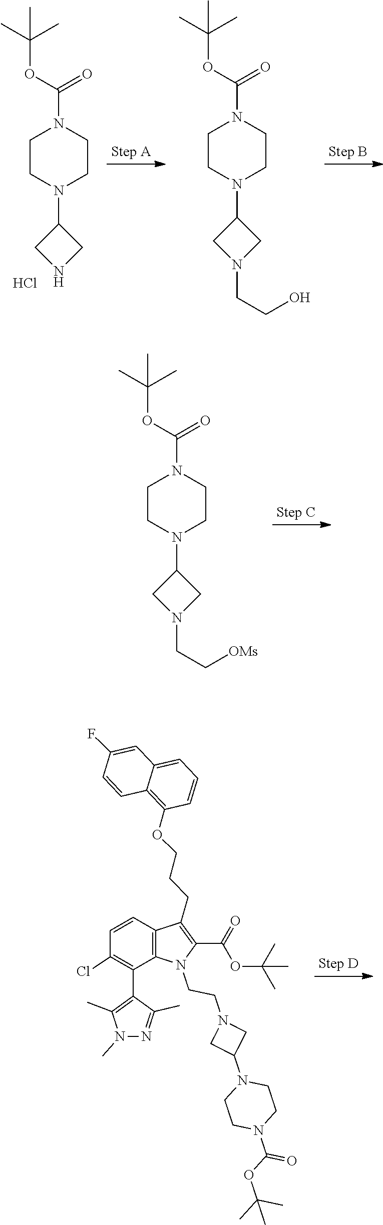 [SMILES: CC(C)(C)OC(=O)N1CCN(C2CNC2)CC1 ; Cl ; CC(C)(C)OC(=O)N1CCN(C2CN(CCO)C2)CC1 ; CC(C)(C)OC(=O)N1CCN(C2CN(CCOS(C)(=O)=O)C2)CC1 ; Cc1nn(C)c(C)c1-c1c(Cl)ccc2c(CCCOc3cccc4cc(F)ccc34)c(C(=O)OC(C)(C)C)n(CCN3CC(N4CCN(C(=O)OC(C)(C)C)CC4)C3)c12]

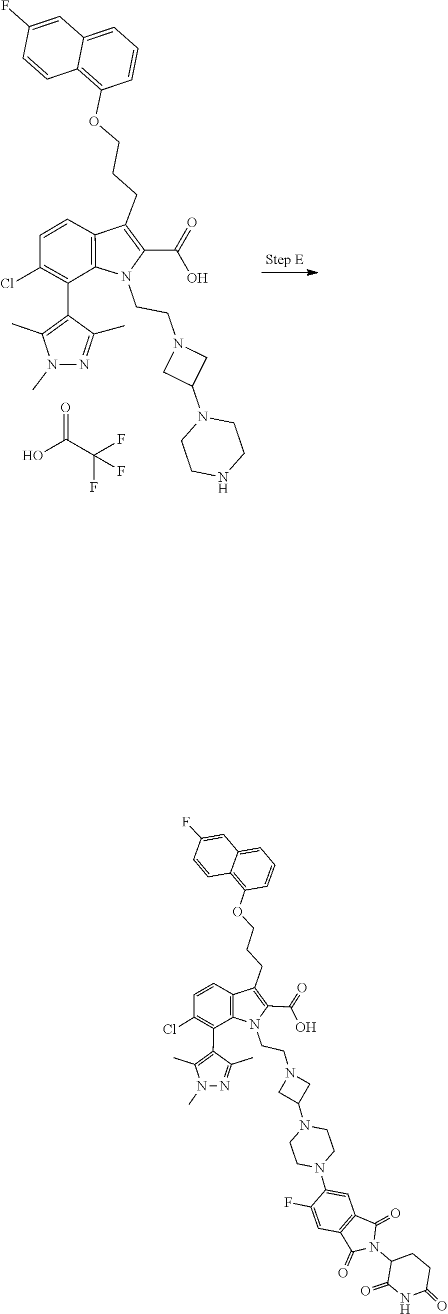 [SMILES: Cc1nn(C)c(C)c1-c1c(Cl)ccc2c(CCCOc3cccc4cc(F)ccc34)c(C(=O)O)n(CCN3CC(N4CCNCC4)C3)c12 ; O=C(O)C(F)(F)F ; Cc1nn(C)c(C)c1-c1c(Cl)ccc2c(CCCOc3cccc4cc(F)ccc34)c(C(=O)O)n(CCN3CC(N4CCN(c5cc6c(cc5F)C(=O)N(C5CCC(=O)NC5=O)C6=O)CC4)C3)c12]

### Step A

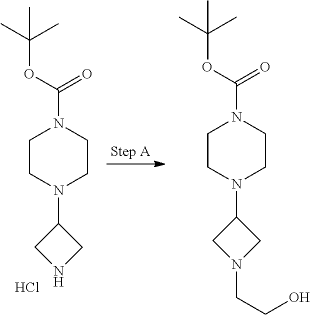 [SMILES: CC(C)(C)OC(=O)N1CCN(C2CNC2)CC1 ; Cl ; CC(C)(C)OC(=O)N1CCN(C2CN(CCO)C2)CC1]

tert-Butyl 4-(azetidin-3-yl)piperazine-1-carboxylate hydrochloride (100.0 mg, 0.360 mmol) and Cs₂CO₃ (469.2 mg, 1.440 mmol) were dissolved in dry DMF (1.8 mL) under argon atmosphere and 2-bromoethanol (0.128 mL, 1.800 mmol) was added. Reaction was stirred at 80° C. for 2 h. After complete consumption of the starting, the mixture was concentrated and the resulting residue dissolved in DCM. Organic phase was washed with brine and water, dried over anhydrous MgSO₄, filtered and concentrated to get crude tert-butyl 4-[1-(2-hydroxyethyl)azetidin-3-yl]piperazine-1-carboxylate (106.2 mg). Obtained product was used in the next step without further purification.

LCMS (ESI+): m/z 286.2 [M+H]⁺

### Step B

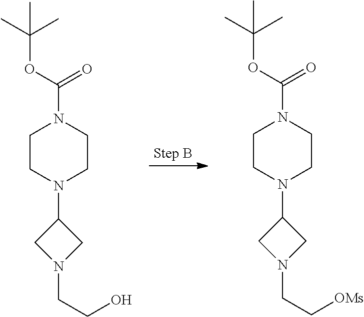 [SMILES: CC(C)(C)OC(=O)N1CCN(C2CN(CCO)C2)CC1 ; CC(C)(C)OC(=O)N1CCN(C2CN(CCOS(C)(=O)=O)C2)CC1]

tert-Butyl 4-[1-(2-hydroxyethyl)azetidin-3-yl]piperazine-1-carboxylate (106.2 mg, crude) was dissolved in dry DCM (3.7 mL) under argon atmosphere and Et₃N (0.078 mL, 0.558 mmol) along with pyridine (0.003 mL) was added. The solution was cooled down to 0° C. and MsCl (0.035 mL, 0.446 mmol) was added. The reaction was allowed to reach room temperature. After 3 h, the solution was diluted with DCM and washed with brine and water. Organic layer was dried over anhydrous MgSO₄, filtered, concentrated and dried under reduced pressure. tert-Butyl 4-{1-[2-(methanesulfonyloxy)ethyl]azetidin-3-yl}piperazine-1-carboxylate (73.2 mg, crude) was obtained as yellow oil and submitted to the next step without further purification.

### Step C

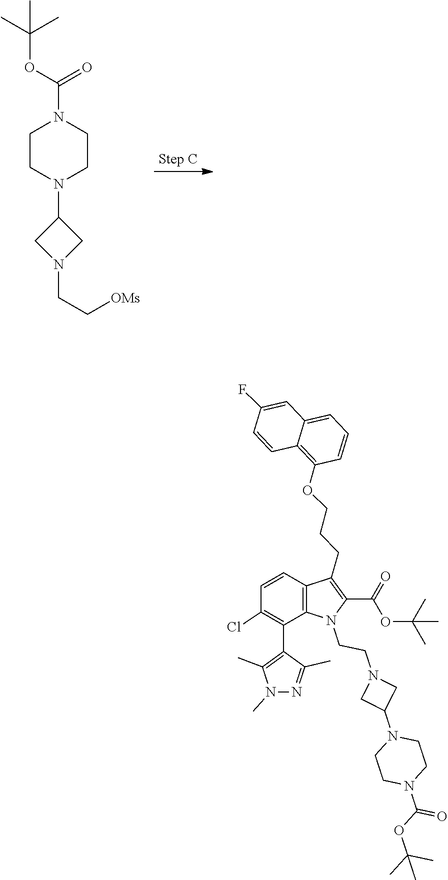 [SMILES: CC(C)(C)OC(=O)N1CCN(C2CN(CCOS(C)(=O)=O)C2)CC1 ; Cc1nn(C)c(C)c1-c1c(Cl)ccc2c(CCCOc3cccc4cc(F)ccc34)c(C(=O)OC(C)(C)C)n(CCN3CC(N4CCN(C(=O)OC(C)(C)C)CC4)C3)c12]

tert-Butyl 6-chloro-3-{3-[(6-fluoronaphthalen-1-yl)oxy]propyl}-7-(1,3,5-trimethyl-1H-pyrazol-4-yl)-1H-indole-2-carboxylate (50.0 mg, 0.089 mmol) was dissolved in dry DMF (1.8 mL) under argon atmosphere and Cs₂CO₃ (86.9 mg, 0.267 mmol) was added. The suspension was stirred for 15 min and tert-butyl 4-{1-[2-(methanesulfonyloxy)ethyl]azetidin-3-yl}piperazine-1-carboxylate (71.1 mg, crude) was added. Reaction was mixed for 96 h at 60° C. under argon atmosphere. Then, additional portion of Cs₂CO₃ (29.0 mg, 0.089 mmol) was added and the reaction stirred at 60° C. for the next 20 h. After no further reaction progress was noted, the mixture was diluted with DCM and washed with brine and water. Organic layer was collected, dried over anhydrous MgSO₄, filtered, concentrated and dried under reduced pressure. The resulting crude tert-butyl 1-[2-(3-{4-[(tert-butoxy)carbonyl]piperazin-1-yl}azetidin-1-yl)ethyl]-6-chloro-3-{3-[(6-fluoronaphthalen-1-yl)oxy]propyl}-7-(1,3,5-trimethyl-1H-pyrazol-4-yl)-1H-indole-2-carboxylate (101.1 mg) was obtained as yellow oil and used in the next step without further purification.

LCMS (ESI+): m/z 829.7 [M+H]⁺

### Step D

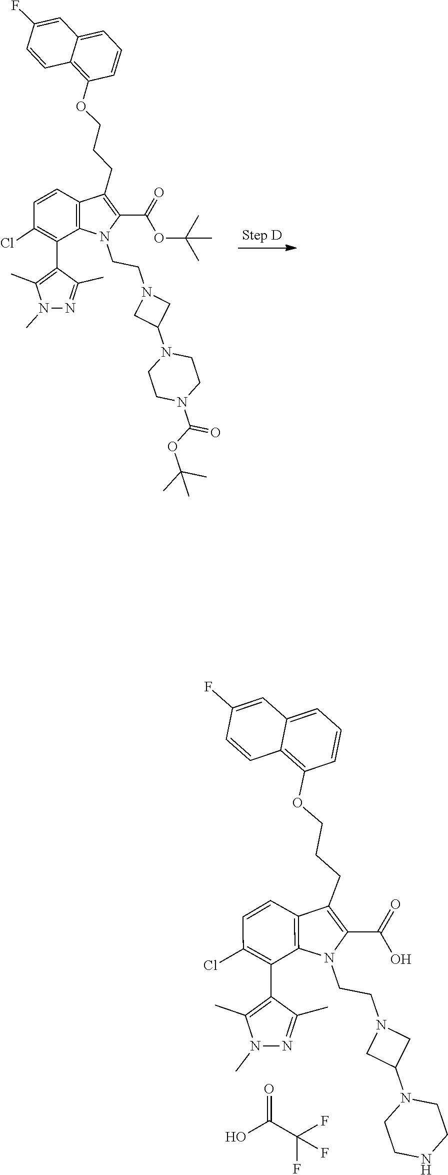 [SMILES: Cc1nn(C)c(C)c1-c1c(Cl)ccc2c(CCCOc3cccc4cc(F)ccc34)c(C(=O)OC(C)(C)C)n(CCN3CC(N4CCN(C(=O)OC(C)(C)C)CC4)C3)c12 ; Cc1nn(C)c(C)c1-c1c(Cl)ccc2c(CCCOc3cccc4cc(F)ccc34)c(C(=O)O)n(CCN3CC(N4CCNCC4)C3)c12 ; O=C(O)C(F)(F)F]

tert-Butyl 1-[2-(3-{4-[(tert-butoxy)carbonyl]piperazin-1-yl}azetidin-1-yl)ethyl]-6-chloro-3-{3-[(6-fluoronaphthalen-1-yl)oxy]propyl}-7-(1,3,5-trimethyl-1H-pyrazol-4-yl)-1H-indole-2-carboxylate (101.1 mg, crude) was dissolved in dry DCM (0.800 mL) under argon atmosphere and TFA (0.800 mL, 10.447 mmol) was added. The reaction was stirred at room temperature for 16 h. After complete conversion of the starting material, the solution was concentrated and dried under reduced pressure. Crude 6-chloro-3-{3-[(6-fluoronaphthalen-1-yl)oxy]propyl}-1-{2-[3-(piperazin-1-yl)azetidin-1-yl]ethyl}-7-(1,3,5-trimethyl-1H-pyrazol-4-yl)-1H-indole-2-carboxylic acid (as TFA salt; 248.2 mg) was used in the next step without further purification.

LCMS (ESI+): m/z 673.8 [M+H]⁺

### Step E

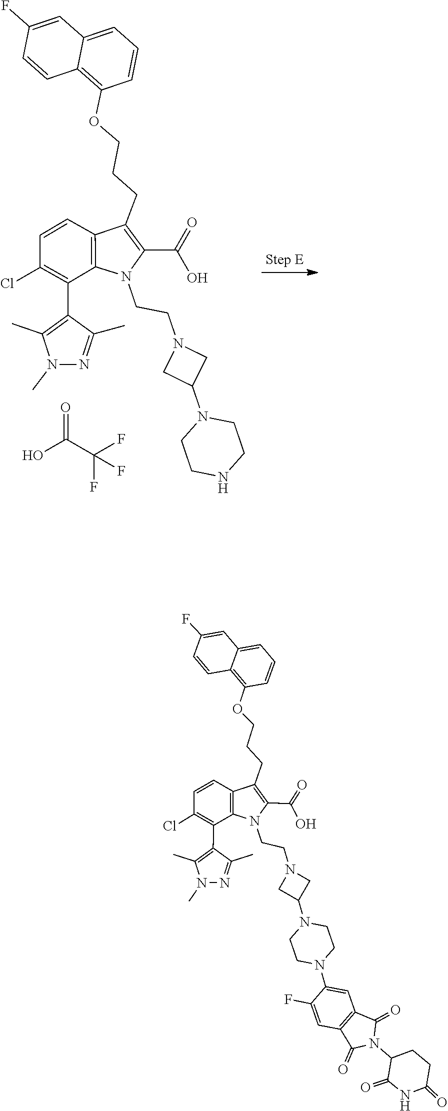 [SMILES: Cc1nn(C)c(C)c1-c1c(Cl)ccc2c(CCCOc3cccc4cc(F)ccc34)c(C(=O)O)n(CCN3CC(N4CCNCC4)C3)c12 ; O=C(O)C(F)(F)F ; Cc1nn(C)c(C)c1-c1c(Cl)ccc2c(CCCOc3cccc4cc(F)ccc34)c(C(=O)O)n(CCN3CC(N4CCN(c5cc6c(cc5F)C(=O)N(C5CCC(=O)NC5=O)C6=O)CC4)C3)c12]

6-Chloro-3-{3-[(6-fluoronaphthalen-1-yl)oxy]propyl}-1-{2-[3-(piperazin-1-yl)azetidin-1-yl]ethyl}-7-(1,3,5-trimethyl-1H-pyrazol-4-yl)-1H-indole-2-carboxylic acid (as TFA salt; 248.8 mg, crude) and 2-(2,6-dioxopiperidin-3-yl)-5,6-difluoro-2,3-dihydro-1H-isoindole-1,3-dione (102.3 mg, 0.348 mmol) were dissolved in dry DMSO (3.2 mL) under argon atmosphere. To it, DIPEA (0.551 mL, 3.161 mmol) was added and the reaction was stirred at 90° C. for 16 h. The solution was cooled down to room temperature and purified with preparative HPLC (C18, H₂O:MeCN+0.1% FA) to give 6-chloro-1-(2-(3-(4-(2-(2,6-dioxopiperidin-3-yl)-6-fluoro-1,3-dioxoisoindolin-5-yl)piperazin-1-yl)azetidin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (6.1 mg, 0.006 mmol, 6.7% over 3 steps) as dark yellow solid.

LCMS (ESI+): m/z 947.3 [M+H]⁺

¹H NMR (500 MHz, DMSO, 353 K) δ 10.78 (s, 1H), 8.23 (dd, J=9.3, 5.9 Hz, 1H), 7.71 (d, J=8.5 Hz, 1H), 7.64-7.52 (m, 2H), 7.45-7.40 (m, 2H), 7.39 (d, J=7.5 Hz, 1H), 7.34-7.27 (m, 1H), 7.23 (d, J=8.5 Hz, 1H), 6.87 (p, J=4.7 Hz, 1H), 5.05 (dd, J=12.5, 5.5 Hz, 1H), 4.25 (t, J=6.4 Hz, 2H), 4.20-4.04 (m, 1H), 3.78 (s, 3H), 3.29-3.25 (m, 2H), 3.24-3.20 (m, 4H), 2.88 (ddd, J=16.7, 13.4, 5.2 Hz, 1H), 2.66-2.54 (m, 3H), 2.48-2.45 (m, 1H), 2.44-2.32 (m, 4H), 2.29-2.20 (m, 2H), 2.14-1.94 (m, 6H), 1.91 (s, 3H).

4 protons in aliphatic area overlap with water.

### Example 107. 6-Chloro-1-[2-(4-{1-[2-(2,6-dioxopiperidin-3-yl)-6-fluoro-1,3-dioxo-2,3-dihydro-1H-isoindol-5-yl]piperidin-4-yl}piperazin-1-yl)ethyl]-3-{3-[(6-fluoronaphthalen-1-yl)oxy]propyl}-7-(1,3,5-trimethyl-1H-pyrazol-4-yl)-1H-indole-2-carboxylic acid (338)

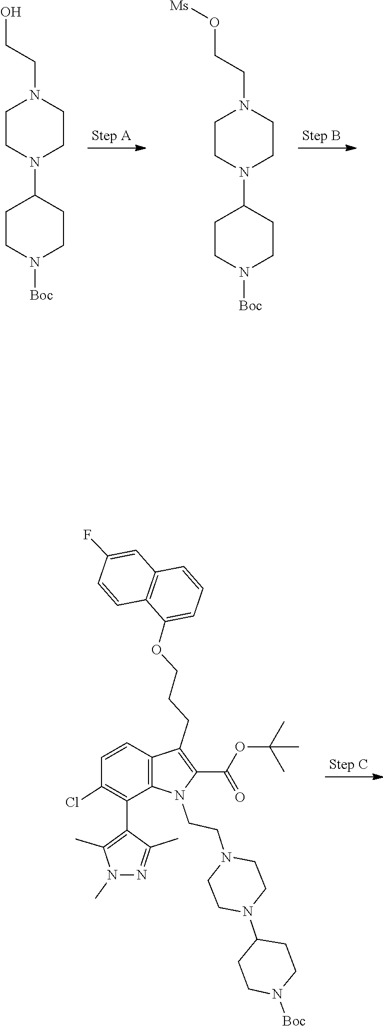 [SMILES: CC(C)(C)OC(=O)N1CCC(N2CCN(CCO)CC2)CC1 ; CC(C)(C)OC(=O)N1CCC(N2CCN(CCOS(C)(=O)=O)CC2)CC1 ; Cc1nn(C)c(C)c1-c1c(Cl)ccc2c(CCCOc3cccc4cc(F)ccc34)c(C(=O)OC(C)(C)C)n(CCN3CCN(C4CCN(C(=O)OC(C)(C)C)CC4)CC3)c12]

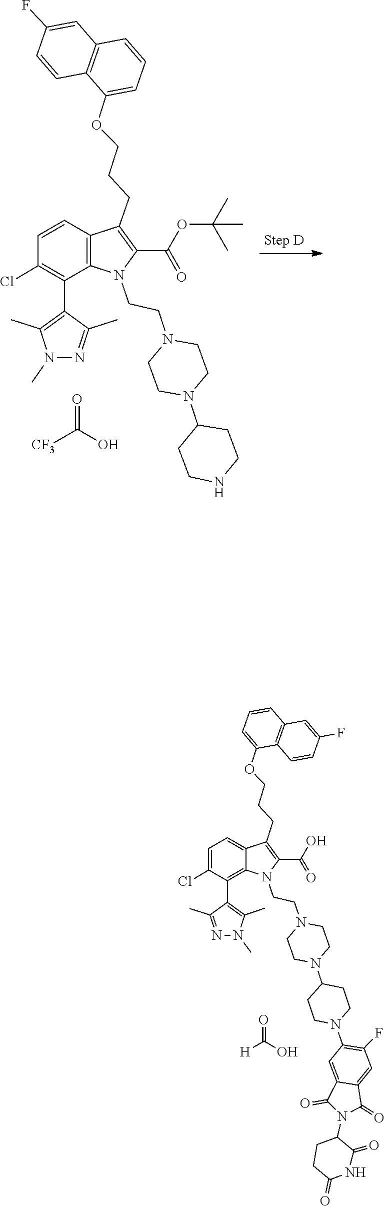

### Step A

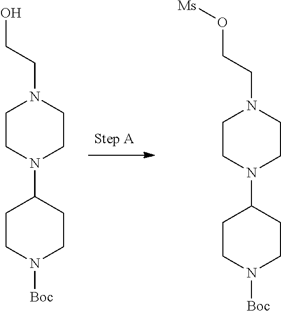 [SMILES: CC(C)(C)OC(=O)N1CCC(N2CCN(CCO)CC2)CC1 ; CC(C)(C)OC(=O)N1CCC(N2CCN(CCOS(C)(=O)=O)CC2)CC1]

tert-Butyl 4-[4-(2-hydroxyethyl)piperazin-1-yl]piperidine-1-carboxylate (87.2 mg, 0.278 mmol) was dissolved in DCM (5.0 mL), Et₃N (0.189 mL, 1.356 mmol) and DMAP (3.3 mg, 0.027 mmol) were added and reaction mixture cooled to 0° C. Then MsCl (0.042 mL, 0.542 mmol) was added dropwise and reaction mixture was let to stir at room temperature for 18 h. The crude was washed with NaHCO₃, water and brine, dried over MgSO₄, filtered and concentrated in vacuo. The isolated product tert-butyl 4-{4-[2-(methanesulfonyloxy)ethyl]piperazin-1-yl}piperidine-1-carboxylate (30 mg, crude) was a yellow oil.

### Step B

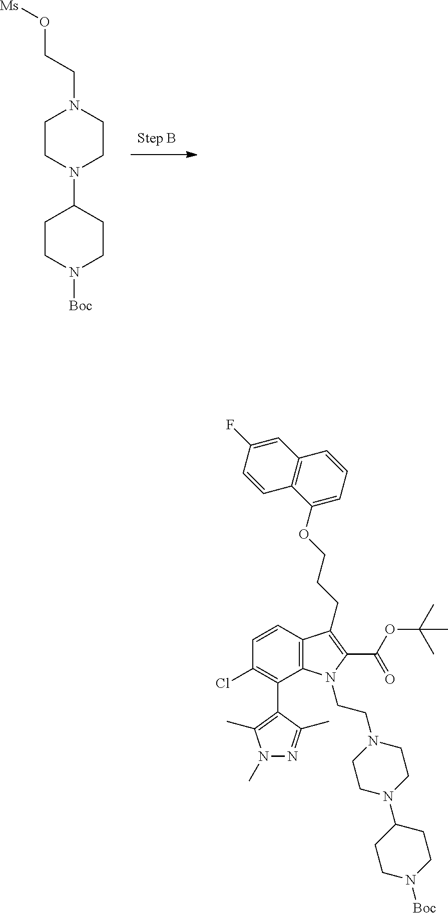 [SMILES: CC(C)(C)OC(=O)N1CCC(N2CCN(CCOS(C)(=O)=O)CC2)CC1 ; Cc1nn(C)c(C)c1-c1c(Cl)ccc2c(CCCOc3cccc4cc(F)ccc34)c(C(=O)OC(C)(C)C)n(CCN3CCN(C4CCN(C(=O)OC(C)(C)C)CC4)CC3)c12]

tert-Butyl 6-chloro-3-{3-[(6-fluoronaphthalen-1-yl)oxy]propyl}-7-(1,3,5-trimethyl-1H-pyrazol-4-yl)-1H-indole-2-carboxylate (25.0 mg, 0.044 mmol), tert-butyl 4-{4-[2-(methanesulfonyloxy)ethyl]piperazin-1-yl}piperidine-1-carboxylate (30 mg, crude) and Cs₂CO₃ (43.5 mg, 0.133 mmol) were dissolved in dry DMF (1.7 mL) in an inert atmosphere and was stirred and stirred at 60° c. for 18 h. After complete consumption of the starting material (monitored by LCMS), the residues were dissolved in DCM and washed with brine. The organic layer was dried over MgSO₄, filtered and concentrated in vacuo to give 40 mg of crude product tert-butyl 1-[2-(4-{1-[(tert-butoxy)carbonyl]piperidin-4-yl}piperazin-1-yl)ethyl]-6-chloro-3-{3-[(6-fluoronaphthalen-1-yl)oxy]propyl}-7-(1,3,5-trimethyl-1H-pyrazol-4-yl)-1H-indole-2-carboxylate, which was used to next step without further purification.

LCMS (ESI+): m/z 857.2 [M+H]⁺

### Step C

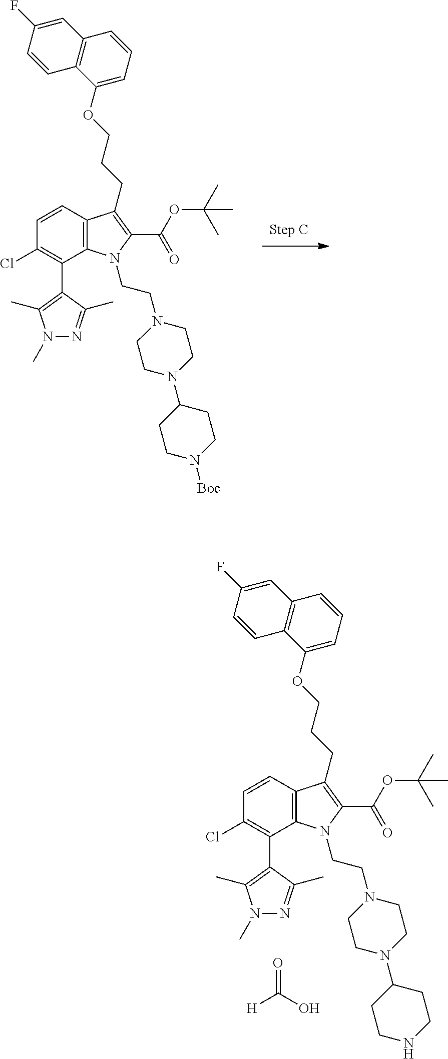 [SMILES: Cc1nn(C)c(C)c1-c1c(Cl)ccc2c(CCCOc3cccc4cc(F)ccc34)c(C(=O)OC(C)(C)C)n(CCN3CCN(C4CCN(C(=O)OC(C)(C)C)CC4)CC3)c12 ; Cc1nn(C)c(C)c1-c1c(Cl)ccc2c(CCCOc3cccc4cc(F)ccc34)c(C(=O)OC(C)(C)C)n(CCN3CCN(C4CCNCC4)CC3)c12 ; O=CO]

tert-Butyl 1-[2-(4-{1-[(tert-butoxy)carbonyl]piperidin-4-yl}piperazin-1-yl)ethyl]-6-chloro-3-{3-[(6-fluoronaphthalen-1-yl)oxy]propyl}-7-(1,3,5-trimethyl-1H-pyrazol-4-yl)-1H-indole-2-carboxylate (40 mg crude) in DCM (1.0 mL) was added TFA (1.5 mL, 19.588 mmol). The reaction was stirred at room temperature for 18 h. The crude was concentrated in vacuo to get 120 mg crude of 6-chloro-3-{3-[(6-fluoronaphthalen-1-yl)oxy]propyl}-1-{2-[4-(piperidin-4-yl)piperazin-1-yl]ethyl}-7-(1,3,5-trimethyl-1H-pyrazol-4-yl)-1H-indole-2-carboxylic acid trifluoroacetate as a yellowish solid, which was used to next step without further purification.

LCMS (ESI+): m/z 701.4 [M+H]⁺

### Step D

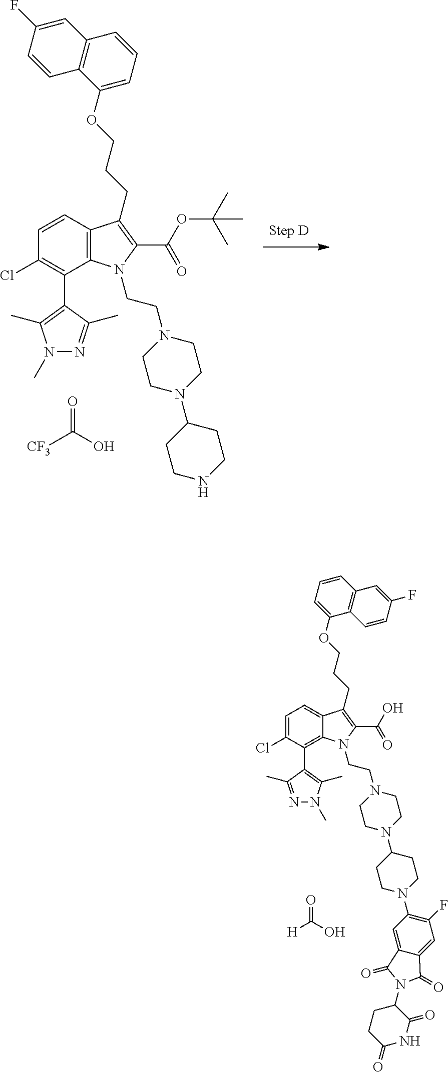 [SMILES: Cc1nn(C)c(C)c1-c1c(Cl)ccc2c(CCCOc3cccc4cc(F)ccc34)c(C(=O)OC(C)(C)C)n(CCN3CCN(C4CCNCC4)CC3)c12 ; O=C(O)C(F)(F)F ; Cc1nn(C)c(C)c1-c1c(Cl)ccc2c(CCCOc3cccc4cc(F)ccc34)c(C(=O)O)n(CCN3CCN(C4CCN(c5cc6c(cc5F)C(=O)N(C5CCC(=O)NC5=O)C6=O)CC4)CC3)c12 ; O=CO]

To a solution of 2-(2,6-dioxopiperidin-3-yl)-5,6-difluoro-2,3-dihydro-1H-isoindole-1,3-dione (8.7 mg, 0.029 mmol) and 6-chloro-3-{3-[(6-fluoronaphthalen-1-yl)oxy]propyl}-1-{2-[4-(piperidin-4-yl)piperazin-1-yl]ethyl}-7-(1,3,5-trimethyl-1H-pyrazol-4-yl)-1H-indole-2-carboxylic acid trifluoroacetate (20 mg crude) in DMSO (2.0 mL) was added DIPEA (0.200 mL, 1.148 mmol) and the mixture was stirred at 90° C. for 18 h. The crude was purified by preparative HPLC (C18, H₂O:MeCN+0.1% FA) to obtain 6-chloro-1-[2-(4-{1-[2-(2,6-dioxopiperidin-3-yl)-6-fluoro-1,3-dioxo-2,3-dihydro-1H-isoindol-5-yl]piperidin-4-yl}piperazin-1-yl)ethyl]-3-{3-[(6-fluoronaphthalen-1-yl)oxy]propyl}-7-(1,3,5-trimethyl-1H-pyrazol-4-yl)-1H-indole-2-carboxylic acid (3.0 mg, 0.003 mmol, 41% yield over 3 steps) as a brownish solid.

LCMS (ESI+): m/z 975.2 [M+H]⁺

¹H NMR (500 MHz, DMSO, 353K) δ 10.77 (s, 1H), 8.25 (dd, J=9.2, 5.9 Hz, 1H), 7.65 (d, J=8.5 Hz, 1H), 7.60-7.56 (m, 2H), 7.43-7.38 (m, 3H), 7.32 (td, J=8.9, 2.7 Hz, 1H), 7.18 (d, J=8.5 Hz, 1H), 6.86 (dd, J=5.4, 3.2 Hz, 1H), 5.05 (dd, J=12.5, 5.5 Hz, 1H), 4.32-4.22 (m, 3H), 4.19-4.12 (m, 1H), 3.76 (s, 3H), 3.62 (d, J=12.4 Hz, 2H), 3.24 (t, J=7.4 Hz, 2H), 2.65-2.52 (m, 2H), 2.42 (t, J=4.8 Hz, 4H), 2.29-1.99 (m, 12H), 1.89 (s, 3H), 1.84-1.76 (m, 2H), 1.59-1.47 (m, 3H).

3 protons in aliphatic area overlap with water.

### Example 108. (S)-6-Chloro-1-(2-(4-((4-(2-(2,6-dioxopiperidin-3-yl)-1-oxoisoindolin-5-yl)piperazin-1-yl)methyl)piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (339)

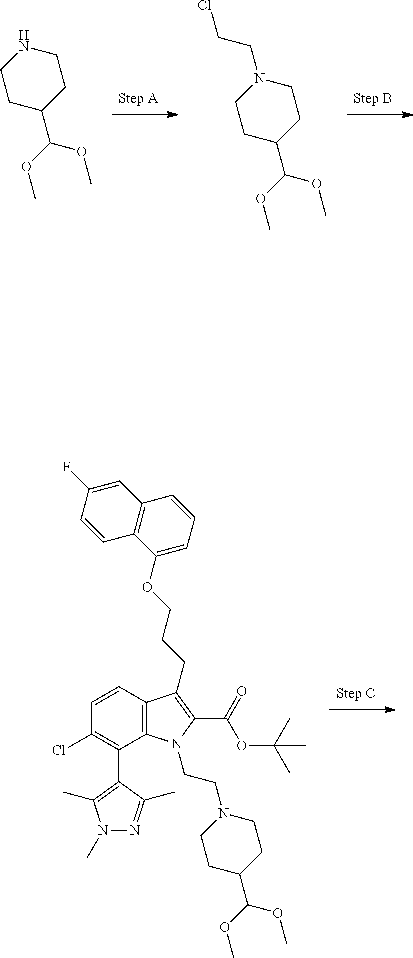 [SMILES: COC(OC)C1CCNCC1 ; COC(OC)C1CCN(CCCl)CC1 ; COC(OC)C1CCN(CCn2c(C(=O)OC(C)(C)C)c(CCCOc3cccc4cc(F)ccc34)c3ccc(Cl)c(-c4c(C)nn(C)c4C)c32)CC1]

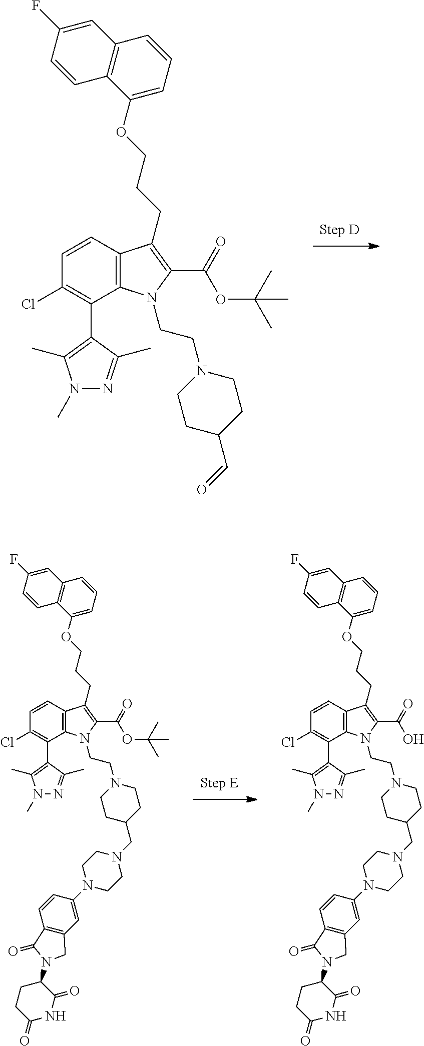

### Step A

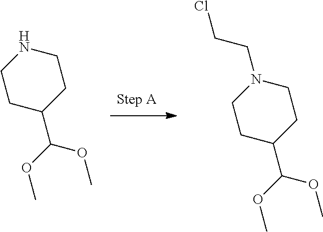

To a stirred solution of 4-(dimethoxymethyl)piperidine (0.4 g, 2.51 mmol) in DMF (10 mL) was added dry (washed with n-hexane) sodium hydride (0.18 g, 7.5 mmol) portion wise at 0° C. under nitrogen. The reaction mixture was allowed to stir at room temperature for 30 minutes after that the reaction mixture was again cooled to 0° C. and was added 1-bromo-2-chloroethane (2.08 mL, 25.1 mmol) dropwise. The resulting reaction mixture was allowed to stir at room temperature under nitrogen for 4 h. The excess sodium hydride was then quenched by addition of ice water and diluted with EtOAc. The organic layer was washed with ice water and brine, dried over Na₂SO₄ and evaporated under reduced pressure. Crude compound was purified with column chromatography (SiO₂, 30-40% EtOAc in hexane) to afford 1-(2-chloroethyl)-4-(dimethoxymethyl)piperidine (300 mg, 1.35 mmol, 54%) as light brown sticky solid.

LCMS (ESI+): m/z 222.2 [M+H]⁺

### Step B

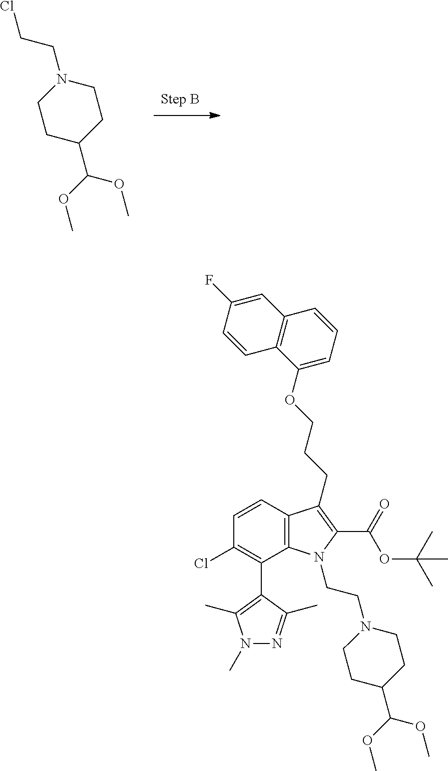 [SMILES: COC(OC)C1CCN(CCCl)CC1 ; COC(OC)C1CCN(CCn2c(C(=O)OC(C)(C)C)c(CCCOc3cccc4cc(F)ccc34)c3ccc(Cl)c(-c4c(C)nn(C)c4C)c32)CC1]

To a well stirred solution of tert-butyl 6-chloro-3-(3-((6-fluoronaphthalen-1-yl)oxy)propyl)-7-(1,3,5-trimethyl-1H-pyrazol-4-yl)-1H-indole-2-carboxylate (350 mg, 0.62 mmol) in DMF (3 mL) was added 1-(2-chloroethyl)-4-(dimethoxymethyl)piperidine (276 mg, 1.24 mmol) followed by Cs₂CO₃ (304 mg, 0.93 mmol) and the mixture was allowed to stir at 100° C. for 16 h under nitrogen. After complete consumption of the starting material (monitored by TLC and LCMS), the reaction mixture was diluted with EtOAc and washed successively with water and brine. The organic layer was dried over Na₂SO₄ and evaporated under reduced pressure to get the crude compound, which was then purified by column chromatography (SiO₂, 60-70% EtOAc in DCM) to get tert-butyl 6-chloro-1-(2-(4-(dimethoxymethyl)piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylate (120 mg, 0.16 mmol, 26%) as light pink solid.

LCMS (ESI+): m/z 747.6 [M+H]⁺

### Step C

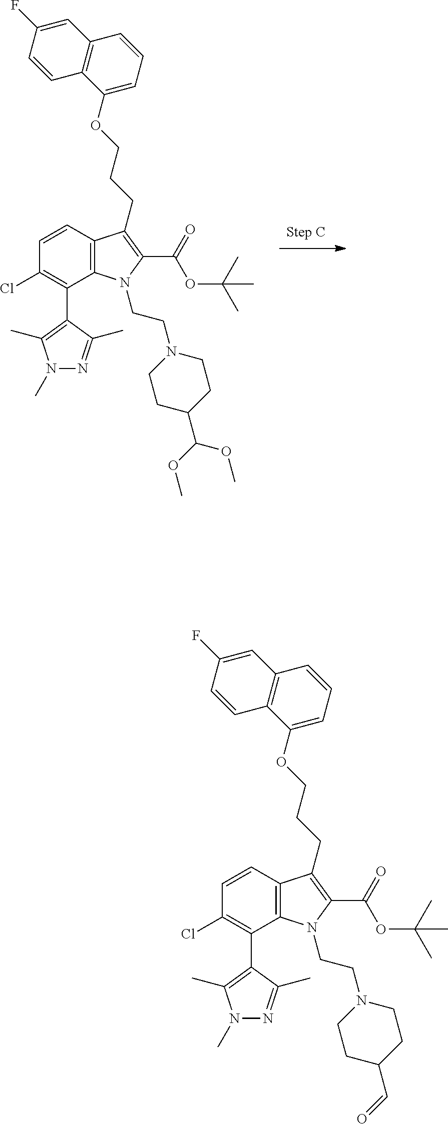 [SMILES: COC(OC)C1CCN(CCn2c(C(=O)OC(C)(C)C)c(CCCOc3cccc4cc(F)ccc34)c3ccc(Cl)c(-c4c(C)nn(C)c4C)c32)CC1 ; Cc1nn(C)c(C)c1-c1c(Cl)ccc2c(CCCOc3cccc4cc(F)ccc34)c(C(=O)OC(C)(C)C)n(CCN3CCC(C=O)CC3)c12]

tert-Butyl 6-chloro-1-{2-[4-(dimethoxymethyl)piperidin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (10.0 mg, 0.013 mmol) was dissolved in THE (0.535 mL) and a 2M aqueous solution of H₂SO₄ (0.268 mL, 0.535 mmol) was added. Reaction was stirred in a sealed vial at 70° C. for 1 h. After complete conversion of the starting material, a solution of 1M NaOH_{aq} was added to pH=7 and reaction product was extracted with DCM. Organic layer was washed with brine, dried over anhydrous MgSO₄, filtered and dried under reduced pressure. Crude tert-butyl 6-chloro-3-{3-[(6-fluoronaphthalen-1-yl)oxy]propyl}-1-[2-(4-formylpiperidin-1-yl)ethyl]-7-(1,3,5-trimethyl-1H-pyrazol-4-yl)-1H-indole-2-carboxylate (7.5 mg) was obtained as off white solid and used directly in the next step.

LCMS (ESI+): m/z 701.4 [M+H]⁺

### Step D

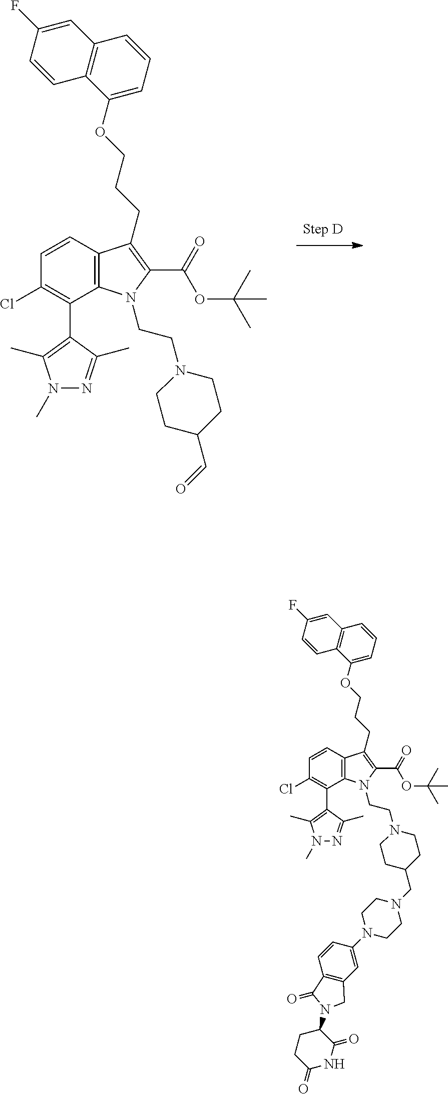 [SMILES: Cc1nn(C)c(C)c1-c1c(Cl)ccc2c(CCCOc3cccc4cc(F)ccc34)c(C(=O)OC(C)(C)C)n(CCN3CCC(C=O)CC3)c12 ; Cc1nn(C)c(C)c1-c1c(Cl)ccc2c(CCCOc3cccc4cc(F)ccc34)c(C(=O)OC(C)(C)C)n(CCN3CCC(CN4CCN(c5ccc6c(c5)CN([C@@H]5CCC(=O)NC5=O)C6=O)CC4)CC3)c12]

Crude tert-butyl 6-chloro-3-{3-[(6-fluoronaphthalen-1-yl)oxy]propyl}-1-[2-(4-formylpiperidin-1-yl)ethyl]-7-(1,3,5-trimethyl-1H-pyrazol-4-yl)-1H-indole-2-carboxylate (7.5 mg) and (3S)-3-[1-oxo-5-(piperazin-1-yl)-2,3-dihydro-1H-isoindol-2-yl]piperidine-2,6-dione (4.8 mg, 0.015 mmol) were dissolved in dry DCM (0.531 mL) and MeOH (0.133 mL) under argon atmosphere. To it, STAB (14.2 mg, 0.067 mmol) and AcOH (0.001 mL, 0.017 mmol) were added, and the reaction was stirred for 16 h under argon at room temperature. After complete consumption of the starting material, the mixture was diluted with DCM and washed with saturated aqueous NaHCO₃, brine and water. Organic layer was dried over anhydrous MgSO₄, filtered and dried under reduced pressure to give tert-butyl (S)-6-chloro-1-(2-(4-((4-(2-(2,6-dioxopiperidin-3-yl)-1-oxoisoindolin-5-yl)piperazin-1-yl)methyl)piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylate (8.4 mg, crude) as beige film. Crude product was submitted to the subsequent step without further purification.

LCMS (ESI+): m/z 1013.3 [M+H]⁺

### Step E

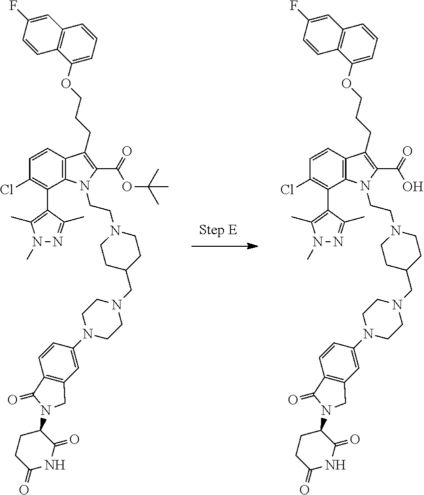 [SMILES: Cc1nn(C)c(C)c1-c1c(Cl)ccc2c(CCCOc3cccc4cc(F)ccc34)c(C(=O)OC(C)(C)C)n(CCN3CCC(CN4CCN(c5ccc6c(c5)CN([C@@H]5CCC(=O)NC5=O)C6=O)CC4)CC3)c12 ; Cc1nn(C)c(C)c1-c1c(Cl)ccc2c(CCCOc3cccc4cc(F)ccc34)c(C(=O)O)n(CCN3CCC(CN4CCN(c5ccc6c(c5)CN([C@@H]5CCC(=O)NC5=O)C6=O)CC4)CC3)c12]

tert-Butyl (S)-6-chloro-1-(2-(4-((4-(2-(2,6-dioxopiperidin-3-yl)-1-oxoisoindolin-5-yl)piperazin-1-yl)methyl)piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylate (8.4 mg, crude) was dissolved under argon atmosphere in dry DCM (0.300 mL) and TFA (0.300 mL, 3.918 mmol) was added. Reaction was stirred for 16 h at room temperature under argon. After complete consumption of the starting material, the solution was concentrated under reduced pressure to dryness and dissolved in DMSO. Crude product was purified with preparative HPLC (C18, H₂O:MeCN+0.1% FA) to get (S)-6-chloro-1-(2-(4-((4-(2-(2,6-dioxopiperidin-3-yl)-1-oxoisoindolin-5-yl)piperazin-1-yl)methyl)piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (2.8 mg, 0.003 mmol, 23% over 3 steps) as white solid.

LCMS (ESI+): m/z 957.25 [M+H]⁺

¹H NMR (500 MHz, DMSO) δ 10.92 (s, 1H), 8.25 (dd, J=9.3, 5.8 Hz, 1H), 7.71-7.59 (m, 2H), 7.50 (d, J=8.6 Hz, 1H), 7.46-7.40 (m, 2H), 7.36 (td, J=8.9, 2.7 Hz, 1H), 7.20 (d, J=8.4 Hz, 1H), 7.05-6.95 (m, 2H), 6.86 (p, J=4.7 Hz, 1H), 5.03 (dd, J=13.3, 5.1 Hz, 1H), 4.31 (d, J=16.8 Hz, 1H), 4.28-4.09 (m, 5H), 3.75 (s, 3H), 3.20-3.15 (m, 6H), 2.89 (ddd, J=18.0, 13.4, 5.4 Hz, 1H), 2.60-2.52 (m, 2H), 2.48-2.30 (m, 10H), 2.19 (p, J=7.0 Hz, 2H), 2.06-1.98 (m, 5H), 1.98-1.92 (m, 1H), 1.88 (s, 3H), 1.65-1.57 (m, 2H), 1.57-1.48 (m, 1H), 1.18-1.06 (m, 2H).

### Example 109: 6-Chloro-1-(2-(4-(4-(2-(2,6-dioxopiperidin-3-yl)-1,3-dioxoisoindolin-4-yl)-2-oxopiperazin-1-yl)piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (340)

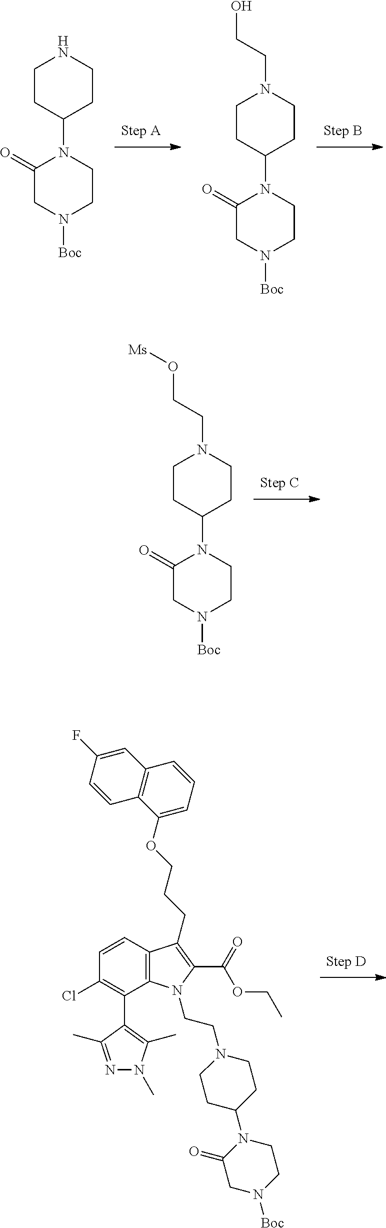 [SMILES: CC(C)(C)OC(=O)N1CCN(C2CCNCC2)C(=O)C1 ; CC(C)(C)OC(=O)N1CCN(C2CCN(CCO)CC2)C(=O)C1 ; CC(C)(C)OC(=O)N1CCN(C2CCN(CCOS(C)(=O)=O)CC2)C(=O)C1 ; CCOC(=O)c1c(CCCOc2cccc3cc(F)ccc23)c2ccc(Cl)c(-c3c(C)nn(C)c3C)c2n1CCN1CCC(N2CCN(C(=O)OC(C)(C)C)CC2=O)CC1]

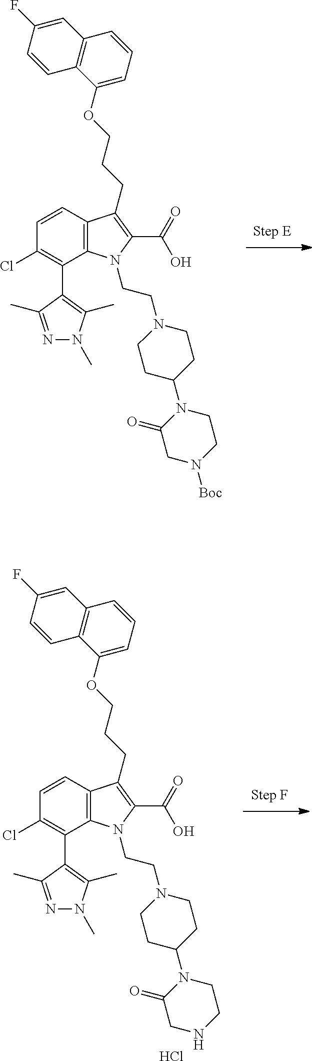 [SMILES: Cc1nn(C)c(C)c1-c1c(Cl)ccc2c(CCCOc3cccc4cc(F)ccc34)c(C(=O)O)n(CCN3CCC(N4CCN(C(=O)OC(C)(C)C)CC4=O)CC3)c12 ; Cc1nn(C)c(C)c1-c1c(Cl)ccc2c(CCCOc3cccc4cc(F)ccc34)c(C(=O)O)n(CCN3CCC(N4CCNCC4=O)CC3)c12 ; Cl]

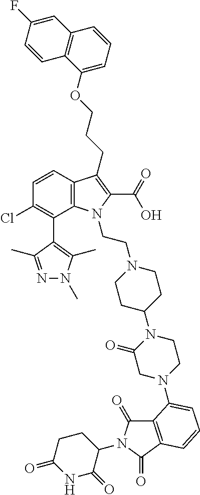 [SMILES: Cc1nn(C)c(C)c1-c1c(Cl)ccc2c(CCCOc3cccc4cc(F)ccc34)c(C(=O)O)n(CCN3CCC(N4CCN(c5cccc6c5C(=O)N(C5CCC(=O)NC5=O)C6=O)CC4=O)CC3)c12]

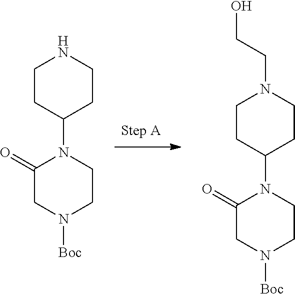 [SMILES: CC(C)(C)OC(=O)N1CCN(C2CCNCC2)C(=O)C1 ; CC(C)(C)OC(=O)N1CCN(C2CCN(CCO)CC2)C(=O)C1]

A mixture of tert-butyl 3-oxo-4-(piperidin-4-yl)piperazine-1-carboxylate (200.0 mg, 0.706 mmol) 2-bromoethanol (250 μL, 3.529 mmol), and K₂CO₃ (390.2 mg, 2.823 mmol) in MeCN (2.0 mL) was stirred at 90° C. under nitrogen atmosphere in a sealed vial for 2 days. Solid was removed by filtration and the filtrate was concentrated under vacuum. To it, a solution of 1M HCl in water was added and the aqueous layer was washed with DCM. To aqueous layer was added 1 M NaOH solution, until pH˜8, and the aqueous layer was extracted with DCM. The organic layer was dried with sodium sulphate and concentrated under vacuum to give tert-butyl 4-[1-(2-hydroxyethyl)piperidin-4-yl]-3-oxopiperazine-1-carboxylate (98.1 mg, 0.300 mmol, 42.5%) as orange oil.

LCMS: (ESI+) m/z 328.2 [M+H]⁺

### Step B

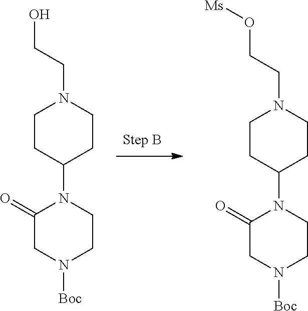 [SMILES: CC(C)(C)OC(=O)N1CCN(C2CCN(CCO)CC2)C(=O)C1 ; CC(C)(C)OC(=O)N1CCN(C2CCN(CCOS(C)(=O)=O)CC2)C(=O)C1]

To a stirred solution of tert-butyl 4-[1-(2-hydroxyethyl)piperidin-4-yl]-3-oxopiperazine-1-carboxylate (95.0 mg, 0.290 mmol) in DCM (1.204 mL) was added DIPEA (3.7 mL, 0.021 mol), followed by mesyl chloride (28 μL, 0.363 mmol) at 0° C. under nitrogen and the reaction mixture was allowed to stir at room temperature for 1 h. After complete consumption of the starting material the reaction mixture was diluted with DCM and washed successively with water and brine. The organic layer was dried over sodium sulphate and evaporated to give tert-butyl 4-{1-[2-(methanesulfonyloxy)ethyl]piperidin-4-yl}-3-oxopiperazine-1-carboxylate (112.0 mg, 0.276 mmol, 95.2%) as orange oil.

LCMS: (ESI+) m/z 406.1 [M+H]⁺

### Step C

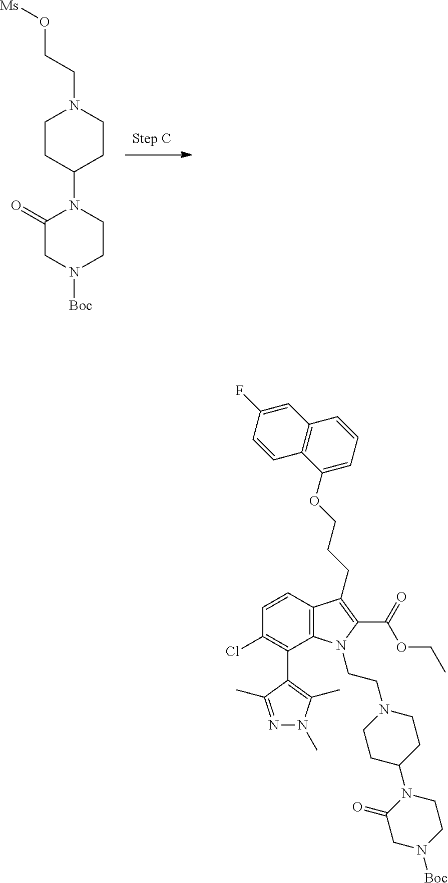 [SMILES: CC(C)(C)OC(=O)N1CCN(C2CCN(CCOS(C)(=O)=O)CC2)C(=O)C1 ; CCOC(=O)c1c(CCCOc2cccc3cc(F)ccc23)c2ccc(Cl)c(-c3c(C)nn(C)c3C)c2n1CCN1CCC(N2CCN(C(=O)OC(C)(C)C)CC2=O)CC1]

Ethyl 6-chloro-3-{3-[(6-fluoronaphthalen-1-yl)oxy]propyl}-7-(1,3,5-trimethyl-1H-pyrazol-4-yl)-1H-indole-2-carboxylate (50.0 mg, 0.094 mmol) was dissolved in MeCN (0.100 mL). Cs₂CO₃ (45.8 mg, 0.140 mmol), tert-butyl 4-{1-[2-(methanesulfonyloxy)ethyl]piperidin-4-yl}-3-oxopiperazine-1-carboxylate (112.0 mg, 0.276 mmol) were added and the reaction was stirred in 70° C. for overnight. Solvent was evaporated, and the residues were dissolved in DCM. Organic layer was washed with water and brine, dried over anhydrous Na₂SO₄, filtered and the solvent was evaporated. The reaction product was dried under reduced pressure, to afford 110 mg of crude ethyl 1-[2-(4-{4-[(tert-butoxy)carbonyl]-2-oxopiperazin-1-yl}piperidin-1-yl)ethyl]-6-chloro-3-{3-[(6-fluoronaphthalen-1-yl)oxy]propyl}-7-(1,3,5-trimethyl-1H-pyrazol-4-yl)-1H-indole-2-carboxylate as yellow oil, which was used for the next step without further purification.

LCMS: (ESI+) m/z 843.7 [M+H]⁺

### Step D

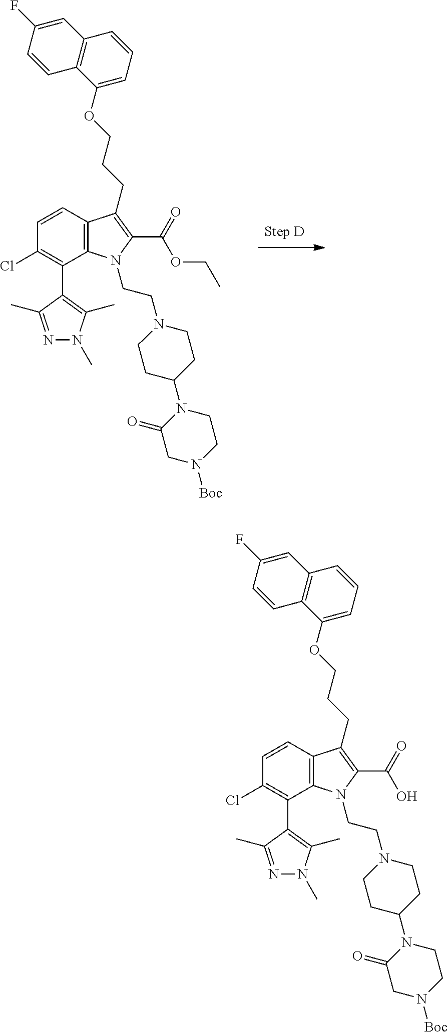 [SMILES: CCOC(=O)c1c(CCCOc2cccc3cc(F)ccc23)c2ccc(Cl)c(-c3c(C)nn(C)c3C)c2n1CCN1CCC(N2CCN(C(=O)OC(C)(C)C)CC2=O)CC1 ; Cc1nn(C)c(C)c1-c1c(Cl)ccc2c(CCCOc3cccc4cc(F)ccc34)c(C(=O)O)n(CCN3CCC(N4CCN(C(=O)OC(C)(C)C)CC4=O)CC3)c12]

To a flask with 110 mg crude of ethyl 1-[2-(4-{4-[(tert-butoxy)carbonyl]-2-oxopiperazin-1-yl}piperidin-1-yl)ethyl]-6-chloro-3-{3-[(6-fluoronaphthalen-1-yl)oxy]propyl}-7-(1,3,5-trimethyl-1H-pyrazol-4-yl)-1H-indole-2-carboxylate in MeOH (1.0 mL) was added solution of 1M LiOH (1.3 mL, 1.304 mmol) and mixture was stirred in room temperature for 24 h. Solvents were removed under reduced pressure. To mixture 1M HCl and DCM were added. The aqueous layer was extracted with DCM. The combined organic layers were dried with sodium sulphate and concentrated under vacuum to afford crude 1-[2-(4-{4-[(tert-butoxy)carbonyl]-2-oxopiperazin-1-yl}piperidin-1-yl)ethyl]-6-chloro-3-{3-[(6-fluoronaphthalen-1-yl)oxy]propyl}-7-(1,3,5-trimethyl-1H-pyrazol-4-yl)-1H-indole-2-carboxylic acid (105 mg) as orange solid, which was used for next step without further purification.

LCMS: (ESI+) m/z 815.9 [M+H]⁺

### Step E

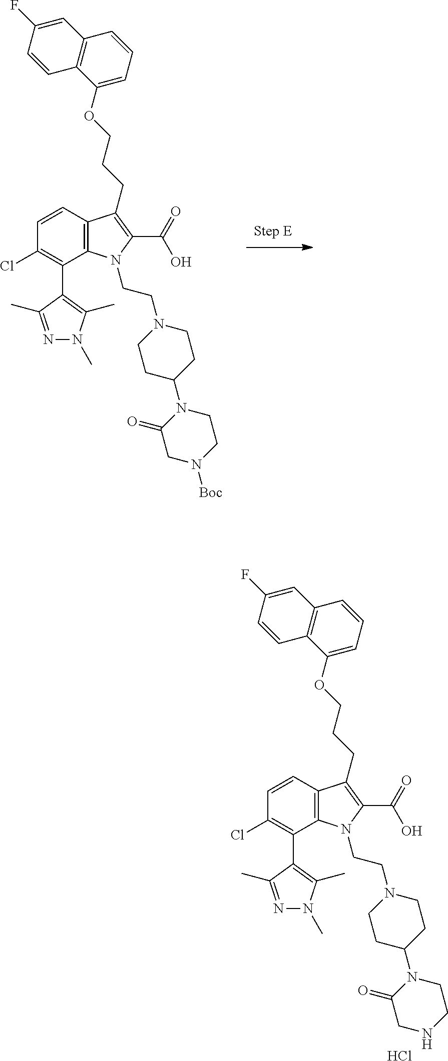 [SMILES: Cc1nn(C)c(C)c1-c1c(Cl)ccc2c(CCCOc3cccc4cc(F)ccc34)c(C(=O)O)n(CCN3CCC(N4CCN(C(=O)OC(C)(C)C)CC4=O)CC3)c12 ; Cc1nn(C)c(C)c1-c1c(Cl)ccc2c(CCCOc3cccc4cc(F)ccc34)c(C(=O)O)n(CCN3CCC(N4CCNCC4=O)CC3)c12 ; Cl]

To a solution of 105 mg crude 1-[2-(4-{4-[(tert-butoxy)carbonyl]-2-oxopiperazin-1-yl}piperidin-1-yl)ethyl]-6-chloro-3-{3-[(6-fluoronaphthalen-1-yl)oxy]propyl}-7-(1,3,5-trimethyl-1H-pyrazol-4-yl)-1H-indole-2-carboxylic acid in THE (1.0 mL) was added 4M HCl in dioxane (0.447 mL, 12.877 mmol). Mixture was stirred at room temperature overnight. After this time MeOH (1.0 mL) was added for solubility, followed by additional portion of 4 M HCl in dioxane (0.447 mL, 12.877 mmol). Mixture was stirred for 2 h at room temperature. Solvents were removed under reduced pressure and crude 6-chloro-3-{3-[(6-fluoronaphthalen-1-yl)oxy]propyl}-1-{2-[4-(2-oxopiperazin-1-yl)piperidin-1-yl]ethyl}-7-(1,3,5-trimethyl-1H-pyrazol-4-yl)-1H-indole-2-carboxylic acid hydrochloride (110 mg) was used for next step without further purification.

LCMS: (ESI+) m/z 715.7 [M+H]⁺

### Step F

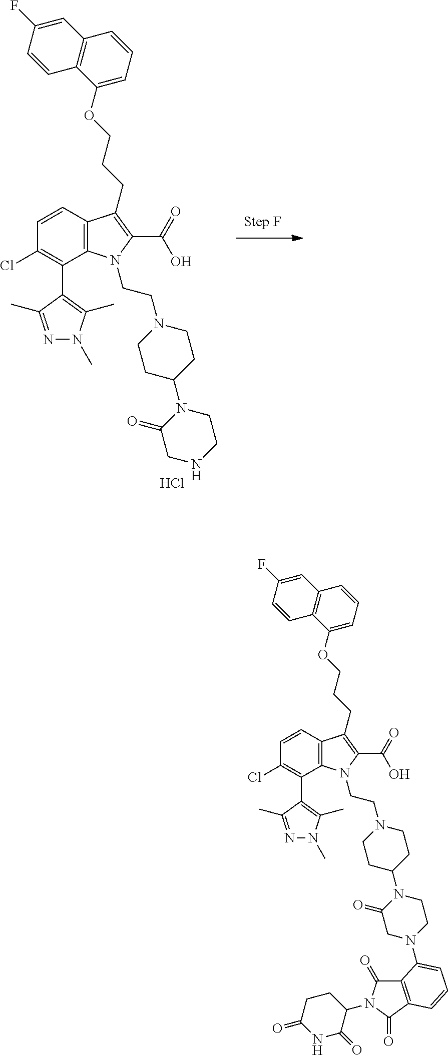 [SMILES: Cc1nn(C)c(C)c1-c1c(Cl)ccc2c(CCCOc3cccc4cc(F)ccc34)c(C(=O)O)n(CCN3CCC(N4CCNCC4=O)CC3)c12 ; Cl ; Cc1nn(C)c(C)c1-c1c(Cl)ccc2c(CCCOc3cccc4cc(F)ccc34)c(C(=O)O)n(CCN3CCC(N4CCN(c5cccc6c5C(=O)N(C5CCC(=O)NC5=O)C6=O)CC4=O)CC3)c12]

To a solution of 2-(2,6-dioxopiperidin-3-yl)-4-fluoro-2,3-dihydro-1H-isoindole-1,3-dione (80.8 mg, 0.293 mmol) in DMSO (3.0 mL) was added 110.0 mg of crude 6-chloro-3-{3-[(6-fluoronaphthalen-1-yl)oxy]propyl}-1-{2-[4-(2-oxopiperazin-1-yl)piperidin-1-yl]ethyl}-7-(1,3,5-trimethyl-1H-pyrazol-4-yl)-1H-indole-2-carboxylic acid hydrochloride followed by DIPEA (0.127 mL, 0.732 mmol). Mixture was heated to 90° C. overnight. After complete consumption of the starting material the solution was cooled down to room temperature, passed through a syringe filter and purified with preparative HPLC (C18, H₂O:MeCN+0.1% FA) to give 6-chloro-1-[2-(4-{4-[2-(2,6-dioxopiperidin-3-yl)-1,3-dioxo-2,3-dihydro-1H-isoindol-4-yl]-2-oxopiperazin-1-yl}piperidin-1-yl)ethyl]-3-{3-[(6-fluoronaphthalen-1-yl)oxy]propyl}-7-(1,3,5-trimethyl-1H-pyrazol-4-yl)-1H-indole-2-carboxylic acid (17.2 mg, 0.018 mmol, yield 18.9% over 4 steps), as yellow solid.

LCMS: (ESI+) m/z 971.2 [M+H]⁺

¹H NMR (500 MHz, DMSO, 353 K) δ 10.77 (s, 1H), 8.24 (dd, J=9.2, 5.9 Hz, 1H), 7.75-7.66 (m, 2H), 7.57 (dd, J=10.4, 2.6 Hz, 1H), 7.45-7.39 (m, 2H), 7.37 (dd, J=7.2, 0.7 Hz, 1H), 7.34-7.27 (m, 2H), 7.21 (d, J=8.5 Hz, 1H), 6.90-6.84 (m, 1H), 5.06 (dd, J=12.6, 5.5 Hz, 1H), 4.30-4.22 (m, 3H), 4.22-4.11 (m, 2H), 3.98 (s, 2H), 3.76 (s, 3H), 3.59 (dd, J=6.3, 4.4 Hz, 2H), 3.39 (dd, J=6.3, 4.4 Hz, 2H), 3.29-3.25 (m, 2H), 2.87 (ddd, J=17.1, 13.6, 5.0 Hz, 1H), 2.66-2.54 (m, 2H), 2.54-2.52 (m, 1H), 2.29-2.21 (m, 2H), 2.20-2.11 (m, 2H), 2.11-2.04 (m, 3H), 2.04-1.99 (m, 4H), 1.89 (s, 3H), 1.75-1.63 (m, 2H), 1.48-1.36 (m, 2H).

### Example 110: 6-Chloro-1-{2-[4-(2-{[2-(2,6-dioxopiperidin-3-yl)-1-oxo-2,3-dihydro-1H-isoindol-5-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (341)

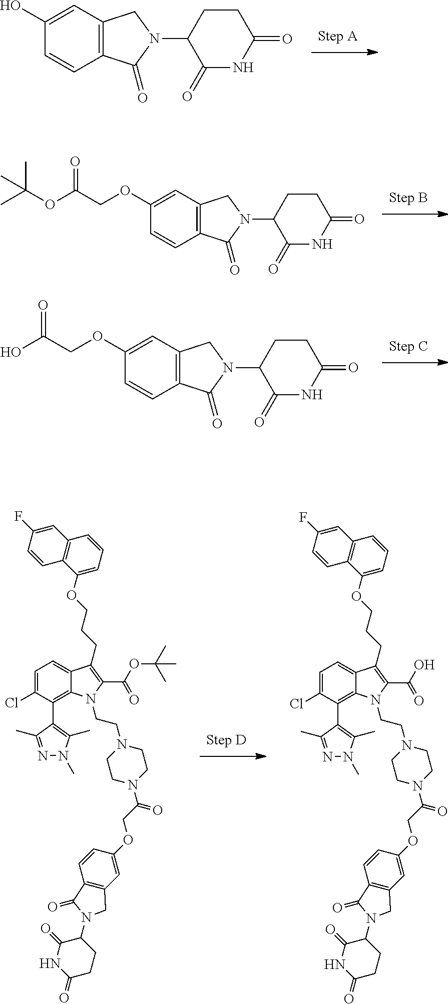 [SMILES: O=C1CCC(N2Cc3cc(O)ccc3C2=O)C(=O)N1 ; CC(C)(C)OC(=O)COc1ccc2c(c1)CN(C1CCC(=O)NC1=O)C2=O ; O=C(O)COc1ccc2c(c1)CN(C1CCC(=O)NC1=O)C2=O ; Cc1nn(C)c(C)c1-c1c(Cl)ccc2c(CCCOc3cccc4cc(F)ccc34)c(C(=O)OC(C)(C)C)n(CCN3CCN(C(=O)COc4ccc5c(c4)CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4ccc5c(c4)CN(C4CCC(=O)NC4=O)C5=O)CC3)c12]

### Step A

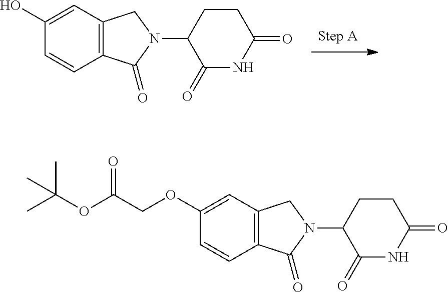 [SMILES: O=C1CCC(N2Cc3cc(O)ccc3C2=O)C(=O)N1 ; CC(C)(C)OC(=O)COc1ccc2c(c1)CN(C1CCC(=O)NC1=O)C2=O]

3-(5-Hydroxy-1-oxo-2,3-dihydro-1H-isoindol-2-yl)piperidine-2,6-dione (100.0 mg, 0.384 mmol) was dissolved in DMF (1.054 mL) and KI (63.8 mg, 0.384 mmol) and KHCO₃ (115.4 mg, 1.153 mmol) were added, followed by dropwise addition of tert-butyl bromoacetate_(57 μL, 0.384 mmol) in DMF (1.054 mL). Reaction was stirred in room temperature for 2 days. Reaction was injected directly to reverse phase flash chromatography column (C18, H₂O:MeCN+0.1% FA) to give a corresponding tert-butyl 2-{[2-(2,6-dioxopiperidin-3-yl)-1-oxo-2,3-dihydro-1H-isoindol-5-yl]oxy}acetate (48.7 mg, 0.130 mmol, 34%), as a white solid.

LCMS (ESI+): m/z 375.1 [M+H]⁺

### Step B

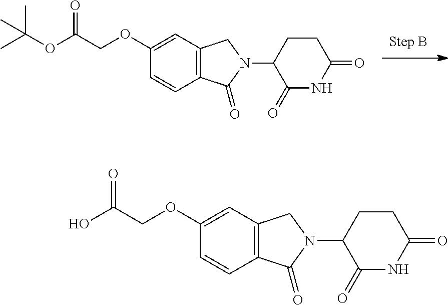 [SMILES: CC(C)(C)OC(=O)COc1ccc2c(c1)CN(C1CCC(=O)NC1=O)C2=O ; O=C(O)COc1ccc2c(c1)CN(C1CCC(=O)NC1=O)C2=O]

tert-Butyl 2-{[2-(2,6-dioxopiperidin-3-yl)-1-oxo-2,3-dihydro-1H-isoindol-5-yl]oxy}acetate (35.1 mg, 0.094 mmol) was dissolved in DCM (0.718 mL), and MeCN (0.800 mL) and TFA (0.717 mL) was added. Mixture was stirred at room temperature for 4 h. Solvents were removed under reduced pressure, and product was co-evaporated with aqueous 1M HCl. 2-{[2-(2,6-dioxopiperidin-3-yl)-1-oxo-2,3-dihydro-1H-isoindol-5-yl]oxy}acetic acid (23.9 mg, 0.075 mmol, 80%) was isolated as white solid.

LCMS (ESI+): m/z 319.2 [M+H]⁺

### Step C

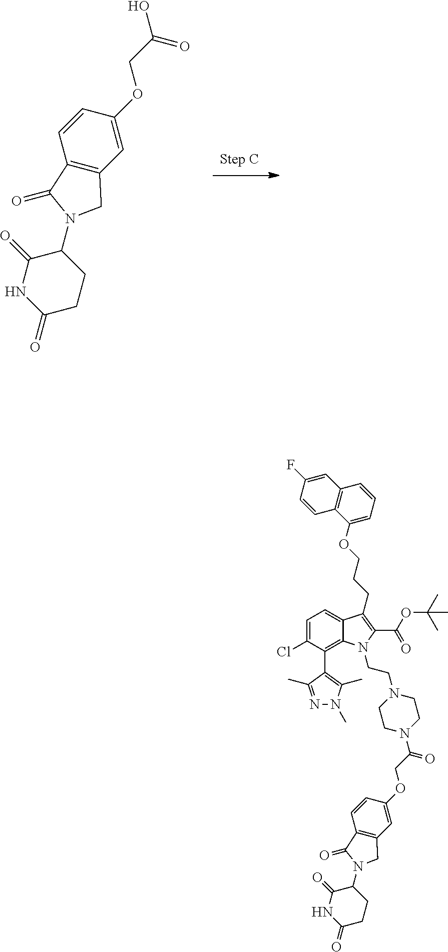 [SMILES: O=C(O)COc1ccc2c(c1)CN(C1CCC(=O)NC1=O)C2=O ; Cc1nn(C)c(C)c1-c1c(Cl)ccc2c(CCCOc3cccc4cc(F)ccc34)c(C(=O)OC(C)(C)C)n(CCN3CCN(C(=O)COc4ccc5c(c4)CN(C4CCC(=O)NC4=O)C5=O)CC3)c12]

2-{[2-(2,6-Dioxopiperidin-3-yl)-1-oxo-2,3-dihydro-1H-isoindol-5-yl]oxy}acetic acid (14.2 mg, 0.044 mmol), tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (25.0 mg, 0.037 mmol) and HATU (18.3 mg, 0.048 mmol) were dissolved in dry DMF (0.882 mL) and to the mixture was added DIPEA (32 μL, 0.185 mmol). The reaction was stirred at room temperature for 18 h, the mixture was diluted with DCM and brine (saturated) was added. The aqueous layer was extracted with DCM. The combined organic layers were dried with Na₂SO₄ and concentrated under vacuum to give tert-butyl 6-chloro-1-{2-[4-(2-{[2-(2,6-dioxopiperidin-3-yl)-1-oxo-2,3-dihydro-1H-isoindol-5-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (20.0 mg, crude) which was used directly to next step.

LCMS (ESI+): m/z 975.0 [M+H]⁺

### Step D

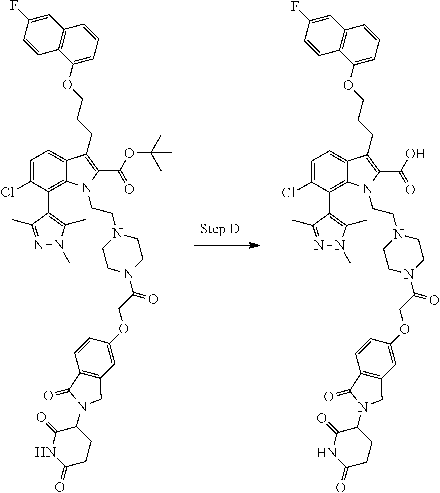 [SMILES: Cc1nn(C)c(C)c1-c1c(Cl)ccc2c(CCCOc3cccc4cc(F)ccc34)c(C(=O)OC(C)(C)C)n(CCN3CCN(C(=O)COc4ccc5c(c4)CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4ccc5c(c4)CN(C4CCC(=O)NC4=O)C5=O)CC3)c12]

To solution of tert-butyl 6-chloro-1-{2-[4-(2-{[2-(2,6-dioxopiperidin-3-yl)-1-oxo-2,3-dihydro-1H-isoindol-5-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (20.0 mg, crude) in DCM (0.676 mL) was added TFA (0.676 mL, 8.831 mmol) and mixture was stirred for 2 days at room temperature. Upon completion solvents were removed under reduced pressure and crude was purified by preparative HPLC (C18, H₂O:MeCN+0.1% FA) to give a corresponding 6-chloro-1-{2-[4-(2-{[2-(2,6-dioxopiperidin-3-yl)-1-oxo-2,3-dihydro-1H-isoindol-5-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (12.0 mg, 0.013 mmol, 35% over two steps) as a white solid.

LCMS (ESI+): m/z 918.15 [M+H]⁺

¹H NMR (500 MHz, DMSO, 353K) δ=10.62 (s, 1H), 8.24 (dd, J=9.3, 5.8, 1H), 7.71 (d, J=8.5, 1H), 7.62 (d, J=8.4, 1H), 7.58 (dd, J=10.4, 2.7, 1H), 7.45-7.37 (m, 2H), 7.32 (td, J=8.9, 2.6, 1H), 7.22 (d, J=8.5, 1H), 7.11 (d, J=2.3, 1H), 7.04 (dd, J=8.4, 2.3, 1H), 6.87 (dd, J=5.9, 2.8, 1H), 5.01 (dd, J=13.0, 5.2, 1H), 4.83 (s, 2H), 4.39 (d, J=16.9, 1H), 4.36-4.14 (m, 5H), 3.76 (s, 3H), 3.42-3.32 (m, 4H), 3.33-3.23 (m, 2H), 2.87 (ddd, J=17.3, 13.5, 5.5, 1H), 2.63 (ddd, J=17.3, 4.5, 2.5, 1H), 2.38 (qd, J=13.1, 4.6, 1H), 2.24 (dq, J=8.6, 6.4, 2H), 2.11 (dt, J=17.6, 6.0, 6H), 2.08-1.98 (m, 4H), 1.89 (s, 3H).

### Example 111. 6-Chloro-1-(2-(4-(2-((2-(2,6-dioxopiperidin-3-yl)-5-fluoro-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (342)

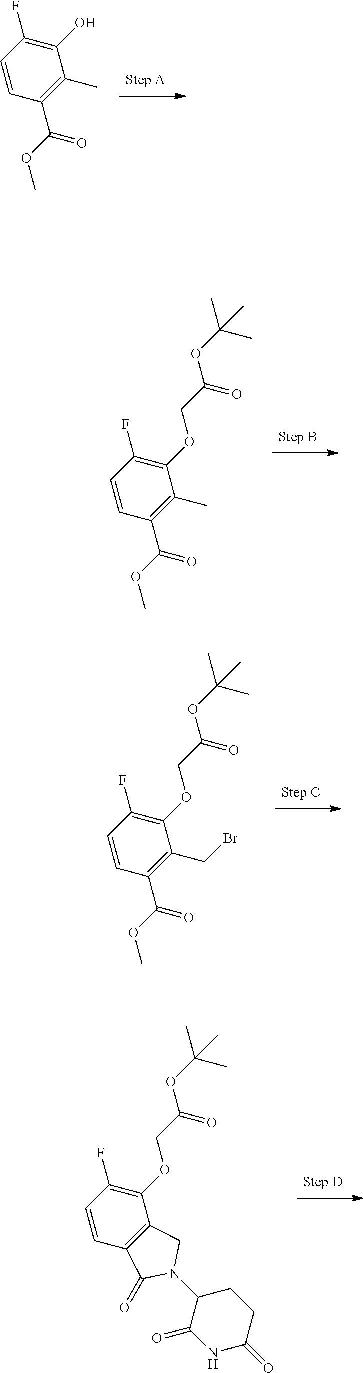 [SMILES: COC(=O)c1ccc(F)c(O)c1C ; COC(=O)c1ccc(F)c(OCC(=O)OC(C)(C)C)c1C ; COC(=O)c1ccc(F)c(OCC(=O)OC(C)(C)C)c1CBr ; CC(C)(C)OC(=O)COc1c(F)ccc2c1CN(C1CCC(=O)NC1=O)C2=O]

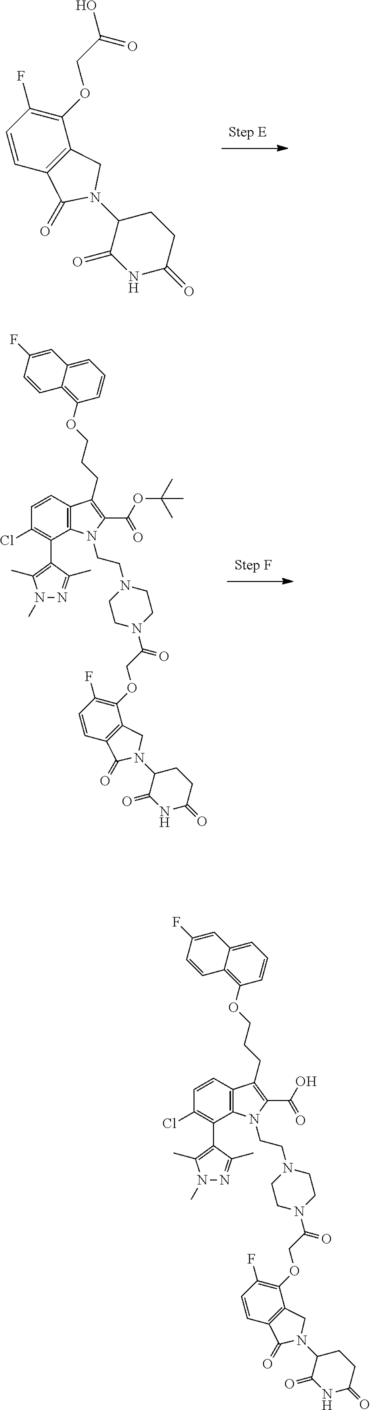 [SMILES: O=C(O)COc1c(F)ccc2c1CN(C1CCC(=O)NC1=O)C2=O ; Cc1nn(C)c(C)c1-c1c(Cl)ccc2c(CCCOc3cccc4cc(F)ccc34)c(C(=O)OC(C)(C)C)n(CCN3CCN(C(=O)COc4c(F)ccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4c(F)ccc5c4CN(C4CCC(=O)NC4=O)C5=O)CC3)c12]

### Step A

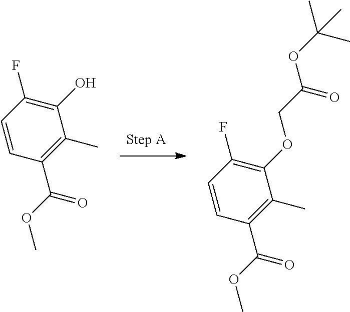 [SMILES: COC(=O)c1ccc(F)c(O)c1C ; COC(=O)c1ccc(F)c(OCC(=O)OC(C)(C)C)c1C]

To a well stirred solution of methyl 4-fluoro-3-hydroxy-2-methylbenzoate (900 mg, 4.891 mmol) in DMF (40 mL) were added KI (405.978 mg, 2.446 mmol) followed by KHCO₃ (978.261 mg, 9.783 mmol). tert-Butyl bromoacetate (1.444 mL, 9.783 mmol) was then added dropwise to the reaction mixture and the resulting reaction mixture was allowed to stir at 60° C. for 24 h under nitrogen. After complete consumption of the starting material (monitored by TLC and LCMS), the reaction mixture was diluted with EtOAc, washed successively with water and brine, the organic layer was dried over anhydrous Na₂SO₄ and evaporated under reduced pressure to get crude compound which was purified by flash column chromatography (SiO₂, 3% EtOAc in hexane) to get methyl 3-(2-(tert-butoxy)-2-oxoethoxy)-4-fluoro-2-methylbenzoate (1.035 g, 3.47 mmol, 70%) as white solid.

GCMS (+): m/z 298.1 [M+H]⁺

### Step B

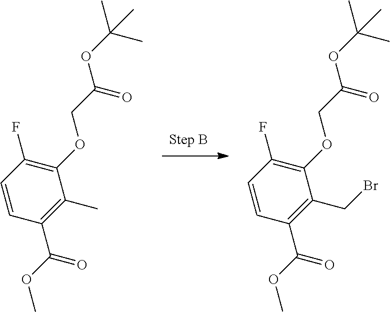 [SMILES: COC(=O)c1ccc(F)c(OCC(=O)OC(C)(C)C)c1C ; COC(=O)c1ccc(F)c(OCC(=O)OC(C)(C)C)c1CBr]

To a stirred solution of methyl 3-(2-(tert-butoxy)-2-oxoethoxy)-4-fluoro-2-methylbenzoate (1.035 g, 3.47 mmol) in dry CCl₄ (40 mL) were added NBS (0.988 g, 5.551 mmol), followed by AIBN (0.114 g, 0.694 mmol) at 0° C. under nitrogen. The reaction mixture was then allowed to stir at 70° C. for 16 h. After complete consumption of the starting material (monitored by TLC and LCMS), the reaction mixture was diluted with EtOAc, washed successively with water and brine, the organic layer was dried over anhydrous Na₂SO₄ and evaporated under reduced pressure to get crude which was purified by flash column chromatography (SiO₂, 3% EtOAc in hexane) to get methyl 2-(bromomethyl)-3-(2-(tert-butoxy)-2-oxoethoxy)-4-fluorobenzoate (1.165 g, 3.09 mmol, 89%) as gummy liquid.

### Step C

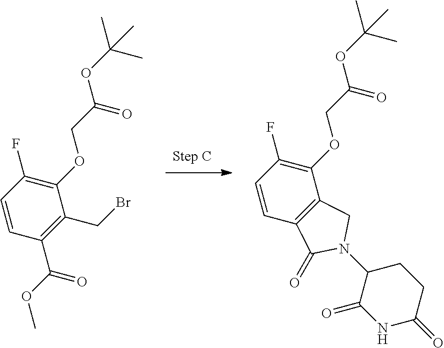 [SMILES: COC(=O)c1ccc(F)c(OCC(=O)OC(C)(C)C)c1CBr ; CC(C)(C)OC(=O)COc1c(F)ccc2c1CN(C1CCC(=O)NC1=O)C2=O]

To a stirred solution of methyl 2-(bromomethyl)-3-(2-(tert-butoxy)-2-oxoethoxy)-4-fluorobenzoate (1.165 g, 3.088 mmol) in MeCN (40.0 mL) were added 3-aminopiperidine-2,6-dione (0.763 g, 4.633 mmol) and DIPEA (1.224 mL, 9.265 mmol) under nitrogen atmosphere. The reaction mixture was stirred at 70° C. for 16 h and monitored by LCMS and TLC. After completion of the reaction, the mixture was concentrated and crude material was purified by flash column chromatography (SiO₂, 50% EtOAc in DCM) to get tert-butyl 2-((2-(2,6-dioxopiperidin-3-yl)-5-fluoro-1-oxoisoindolin-4-yl)oxy)acetate (450 mg, 1.15 mmol, 37%) as brown solid.

LCMS (ESI+): m/z 392.9 [M+H]⁺

### Step D

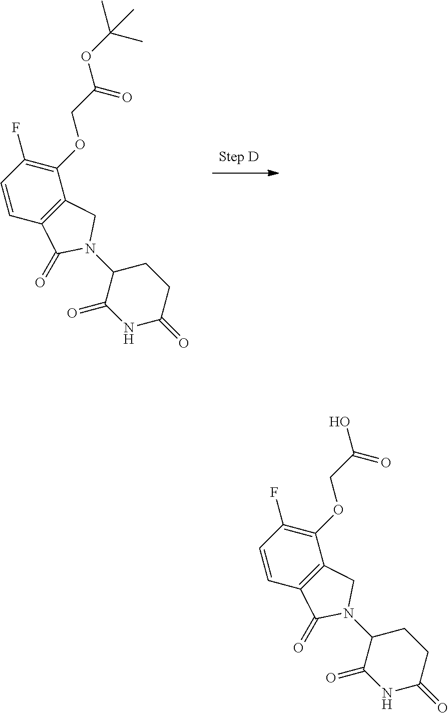 [SMILES: CC(C)(C)OC(=O)COc1c(F)ccc2c1CN(C1CCC(=O)NC1=O)C2=O ; O=C(O)COc1c(F)ccc2c1CN(C1CCC(=O)NC1=O)C2=O]

To a stirred solution of tert-butyl 2-((2-(2,6-dioxopiperidin-3-yl)-5-fluoro-1-oxoisoindolin-4-yl)oxy)acetate as brown solid (450.0 mg, 1.147 mmol) in DCM (12.0 mL) was added TFA (6.0 mL) at 0° C. dropwise to the reaction mixture. The reaction mixture was allowed to stir at ambient temperature for 16 h under nitrogen. After complete consumption of the starting material (monitored by TLC and LCMS) the volatiles were evaporated under reduced pressure. The crude compound was then purified by triturating it with diethyl ether and pentane to afford (350 mg, 90%, 1.04 mmol) of the target molecule as light purple solid.

LCMS (ESI+): m/z 337.2 [M+H]⁺

### Step E

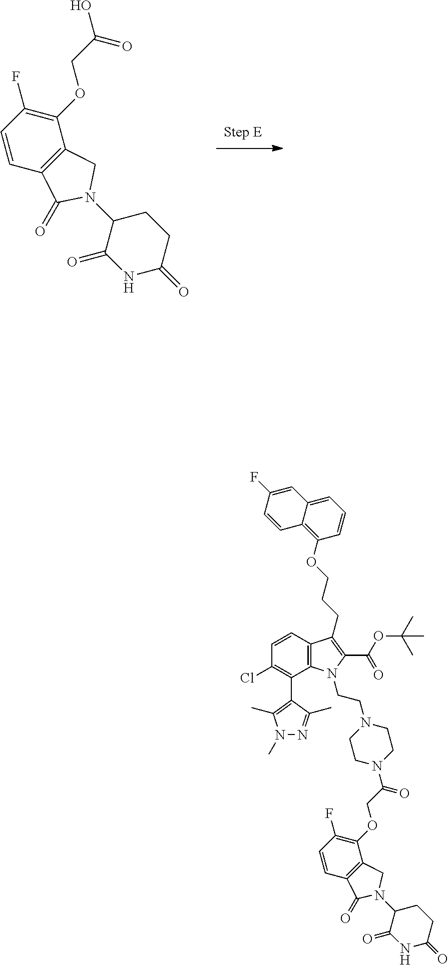 [SMILES: O=C(O)COc1c(F)ccc2c1CN(C1CCC(=O)NC1=O)C2=O ; Cc1nn(C)c(C)c1-c1c(Cl)ccc2c(CCCOc3cccc4cc(F)ccc34)c(C(=O)OC(C)(C)C)n(CCN3CCN(C(=O)COc4c(F)ccc5c4CN(C4CCC(=O)NC4=O)C5=O)CC3)c12]

2-{[2-(2,6-Dioxopiperidin-3-yl)-5-fluoro-1-oxo-2,3-dihydro-1H-isoindol-4-yl]oxy}acetic acid (29.9 mg, 0.089 mmol) was dissolved in dry DMF under argon atmosphere and HATU (29.6 mg, 0.078 mmol) along with DIPEA (0.039 mL, 0.222 mmol) were added and the reaction was stirred for 10 min. To it, tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (50.0 mg, 0.074 mmol) was added and the reaction (monitored with LCMS) was continued in room temperature under argon for 15 min. After complete consumption of the starting material, the solution was diluted with DCM and washed with brine and water. The organic layer was dried over anhydrous MgSO₄, filtered and dried under reduced pressure. Crude tert-butyl 6-chloro-1-(2-(4-(2-((2-(2,6-dioxopiperidin-3-yl)-5-fluoro-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (80.3 mg) was obtained as yellow oil and used in the next step without further purification.

LCMS (ESI+): m/z 992.6 [M+H]⁺

### Step F

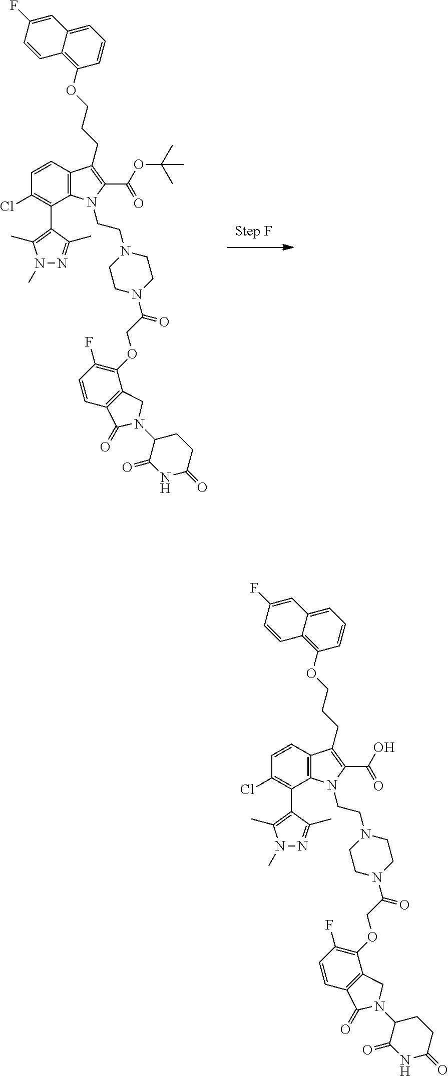 [SMILES: Cc1nn(C)c(C)c1-c1c(Cl)ccc2c(CCCOc3cccc4cc(F)ccc34)c(C(=O)OC(C)(C)C)n(CCN3CCN(C(=O)COc4c(F)ccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4c(F)ccc5c4CN(C4CCC(=O)NC4=O)C5=O)CC3)c12]

tert-Butyl 6-chloro-1-(2-(4-(2-((2-(2,6-dioxopiperidin-3-yl)-5-fluoro-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (80.3 mg, 0.081 mmol) was dissolved in dry DCM (0.700 mL) under argon atmosphere and TFA (0.700 mL, 9.141 mmol) was added. Reaction (monitored with LCMS) was stirred for 16 h in room temperature. After complete consumption of the starting material the mixture was concentrated under reduced pressure and the resulting residue was dissolved in DMSO and passed through a syringe filter. Crude product was purified with preparative HPLC (C18, H₂O:MeCN+0.1% FA) to give 6-chloro-1-(2-(4-(2-((2-(2,6-dioxopiperidin-3-yl)-5-fluoro-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (30.6 mg, 0.033 mmol, 44.6% over 2 steps) as white solid.

LCMS (ESI+): m/z 936.3 [M+H]⁺

¹H NMR (500 MHz, DMSO, 353K) δ 10.69 (s, 1H), 8.24 (dd, J=9.2, 5.9 Hz, 1H), 7.71 (d, J=8.6 Hz, 1H), 7.59 (dd, J=10.4, 2.6 Hz, 1H), 7.45-7.38 (m, 3H), 7.38-7.29 (m, 2H), 7.22 (d, J=8.5 Hz, 1H), 6.87 (dd, J=5.8, 2.8 Hz, 1H), 5.03 (dd, J=13.0, 5.2 Hz, 1H), 4.94 (s, 2H), 4.53 (d, J=16.9 Hz, 1H), 4.45 (dd, J=17.4, 2.1 Hz, 1H), 4.31-4.21 (m, 3H), 4.21-4.13 (m, 1H), 3.76 (s, 3H), 3.36-3.30 (m, 4H), 3.30-3.25 (m, 2H), 2.88 (ddd, J=17.7, 13.5, 5.5 Hz, 1H), 2.66-2.59 (m, 1H), 2.48-2.37 (m, 1H), 2.28-2.19 (m, 2H), 2.15-2.09 (m, 5H), 2.09-2.02 (m, 2H), 2.01 (s, 3H), 1.88 (s, 3H).

### Example 112. 6-Chloro-1-(2-(4-(2-((2-(2,6-dioxopiperidin-3-yl)-6-fluoro-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (343)

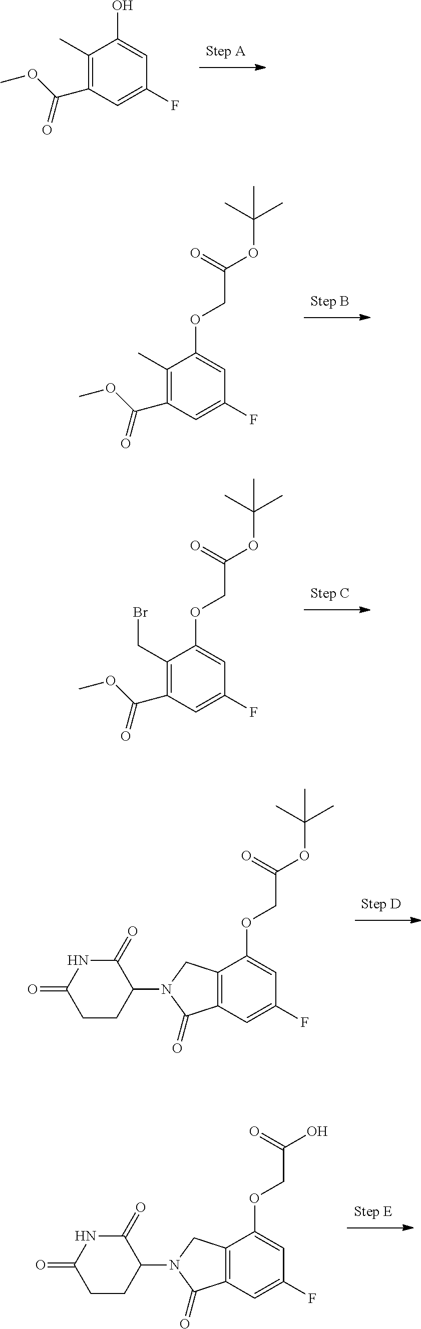

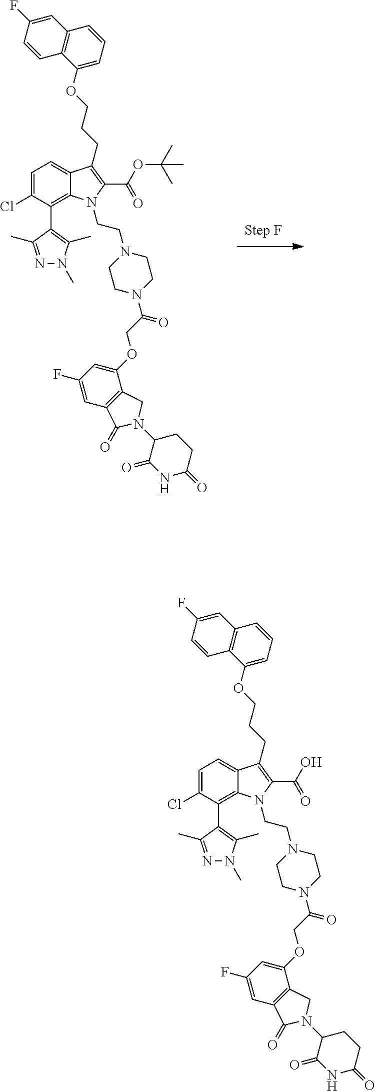 [SMILES: Cc1nn(C)c(C)c1-c1c(Cl)ccc2c(CCCOc3cccc4cc(F)ccc34)c(C(=O)OC(C)(C)C)n(CCN3CCN(C(=O)COc4cc(F)cc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4cc(F)cc5c4CN(C4CCC(=O)NC4=O)C5=O)CC3)c12]

### Step A

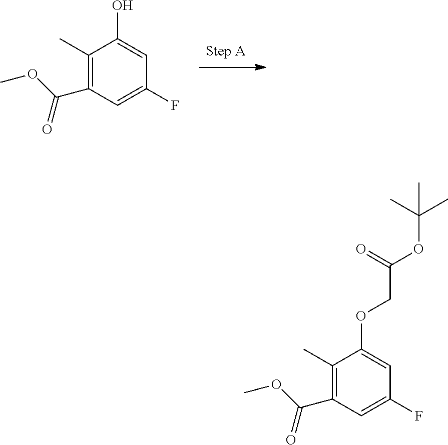 [SMILES: COC(=O)c1cc(F)cc(O)c1C ; COC(=O)c1cc(F)cc(OCC(=O)OC(C)(C)C)c1C]

To a well stirred solution of crude methyl 5-fluoro-3-hydroxy-2-methylbenzoate (4.0 g, 21.7 mmol) in DMF (40 mL) were added KI (1.13 g, 6.8 mmol) followed by KHCO₃ (2.72 g, 27.16 mmol). Tert-butyl bromoacetate (4.1 mL, 27.2 mmol) was then added dropwise to the reaction and the resulting mixture was allowed to stir at 60° C. for 24 h under nitrogen. After complete consumption of the starting material (monitored by TLC and LCMS), the reaction mixture was diluted with EtOAc, washed successively with cold water and brine. The organic layer was dried over anhydrous Na₂SO₄ and evaporated under reduced pressure to get crude material which was purified with flash column chromatography (SiO₂, 3% EtOAc in hexane) to get methyl 3-(2-(tert-butoxy)-2-oxoethoxy)-5-fluoro-2-methylbenzoate (2.3 g, 7.71 mmol, 35.5%) of as white solid.

LCMS (ESI+): m/z 298.2 [M+H]⁺

### Step B

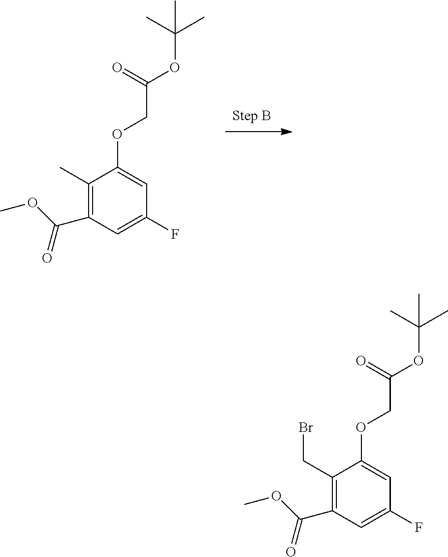 [SMILES: COC(=O)c1cc(F)cc(OCC(=O)OC(C)(C)C)c1C ; COC(=O)c1cc(F)cc(OCC(=O)OC(C)(C)C)c1CBr]

To a solution of methyl 3-(2-(tert-butoxy)-2-oxoethoxy)-5-fluoro-2-methylbenzoate (2.3 g, 7.71 mmol) in dry CCl₄ (100.0 mL) were added NBS (1.78 g, 10.02 mmol) followed by AIBN (0.255 g, 1.54 mmol) at 0° C. under nitrogen. The reaction mixture was then allowed to stir at 70° C. for 16 h. After complete consumption of the starting material (monitored by TLC and LCMS), the reaction mixture was diluted with EtOAc and washed successively with water and brine. The organic layer was dried over anhydrous Na₂SO₄ and evaporated under reduced pressure to get crude which was purified by flash column chromatography (SiO₂, 3% EtOAc in hexane) to get methyl 2-(bromomethyl)-3-(2-(tert-butoxy)-2-oxoethoxy)-5-fluorobenzoate (2.5 g, 6.63 mmol, 86%) as gummy liquid.

### Step C

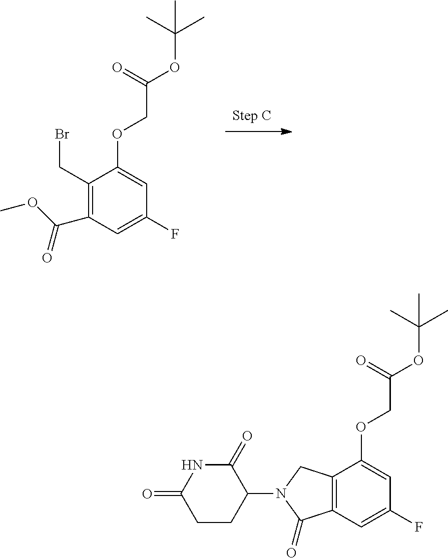 [SMILES: COC(=O)c1cc(F)cc(OCC(=O)OC(C)(C)C)c1CBr ; CC(C)(C)OC(=O)COc1cc(F)cc2c1CN(C1CCC(=O)NC1=O)C2=O]

To a stirred solution of methyl 2-(bromomethyl)-3-(2-(tert-butoxy)-2-oxoethoxy)-5-fluorobenzoate (2.5 g, 6.63 mmol) in MeCN (80.0 mL) were added 3-aminopiperidine-2,6-dione hydrochloride (1.53 g, 9.3 mmol) and DIPEA (2.7 mL, 19.9 mmol) under nitrogen atmosphere. The reaction mixture was stirred at 70° C. for 16 h. After reaction completion, the mixture was concentrated and crude material was purified with flash column chromatography (SiO₂, 50% EtOAc in DCM) to get tert-butyl 2-((2-(2,6-dioxopiperidin-3-yl)-6-fluoro-1-oxoisoindolin-4-yl)oxy)acetate (1.2 g, 3.06 mmol, 46%) as brown solid.

LCMS (ESI+): m/z 393.2 [M+H]⁺

### Step D

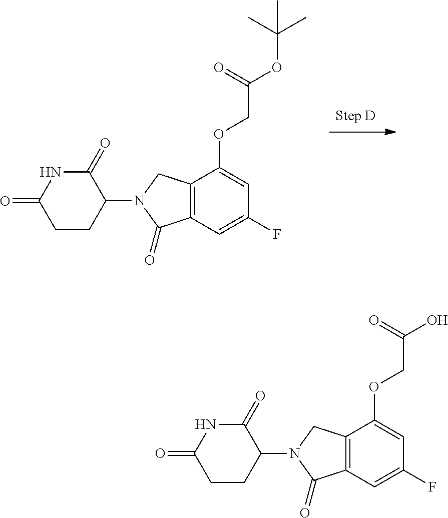 [SMILES: CC(C)(C)OC(=O)COc1cc(F)cc2c1CN(C1CCC(=O)NC1=O)C2=O ; O=C(O)COc1cc(F)cc2c1CN(C1CCC(=O)NC1=O)C2=O]

To a stirred solution of tert-butyl 2-((2-(2,6-dioxopiperidin-3-yl)-6-fluoro-1-oxoisoindolin-4-yl)oxy)acetate (1.2 g, 3.06 mmol) in DCM (42.0 mL) was added TFA (18.0 mL) at 0° C. dropwise and the reaction mixture was allowed to stir at ambient temperature for 16 h under nitrogen. After complete consumption of the starting material (monitored by TLC and LCMS), the volatiles were evaporated under reduced pressure to get the crude compound which was then purified by triturating the reaction mass with Et₂O and pentane to afford 2-((2-(2,6-dioxopiperidin-3-yl)-6-fluoro-1-oxoisoindolin-4-yl)oxy)acetic acid (1.0 g, 2.97 mmol, 97%) as off white solid.

LCMS (ESI+): m/z 336.95 [M+H]⁺

### Step E

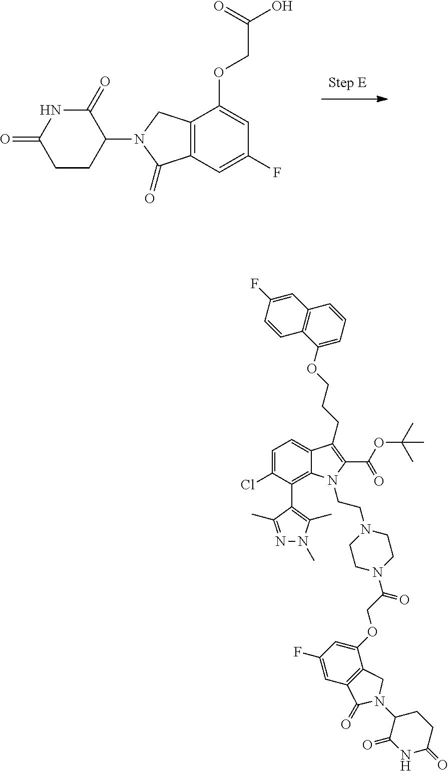 [SMILES: O=C(O)COc1cc(F)cc2c1CN(C1CCC(=O)NC1=O)C2=O ; Cc1nn(C)c(C)c1-c1c(Cl)ccc2c(CCCOc3cccc4cc(F)ccc34)c(C(=O)OC(C)(C)C)n(CCN3CCN(C(=O)COc4cc(F)cc5c4CN(C4CCC(=O)NC4=O)C5=O)CC3)c12]

2-{[2-(2,6-Dioxopiperidin-3-yl)-6-fluoro-1-oxo-2,3-dihydro-1H-isoindol-4-yl]oxy}acetic acid (13.7 mg, 0.041 mmol) and tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (25.0 mg, 0.037 mmol) were dissolved in dry DMF (1.0 mL) and DIPEA (0.019 mL, 0.111 mmol) was added under argon atmosphere. To it, HATU (14.8 mg, 0.039 mmol) was added dropwise in DMF (0.2 mL) and the reaction was stirred at room temperature under argon atmosphere for 20 min. After complete consumption of the starting material, the mixture was diluted with DCM and washed with brine and water. Organic layer was dried over anhydrous MgSO₄, filtered and concentrated under reduced pressure to give 45.4 mg crude of tert-butyl 6-chloro-1-{2-[4-(2-{[2-(2,6-dioxopiperidin-3-yl)-6-fluoro-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate as an yellow oil.

LCMS (ESI+): m/z 992.4 [M+H]⁺

### Step F

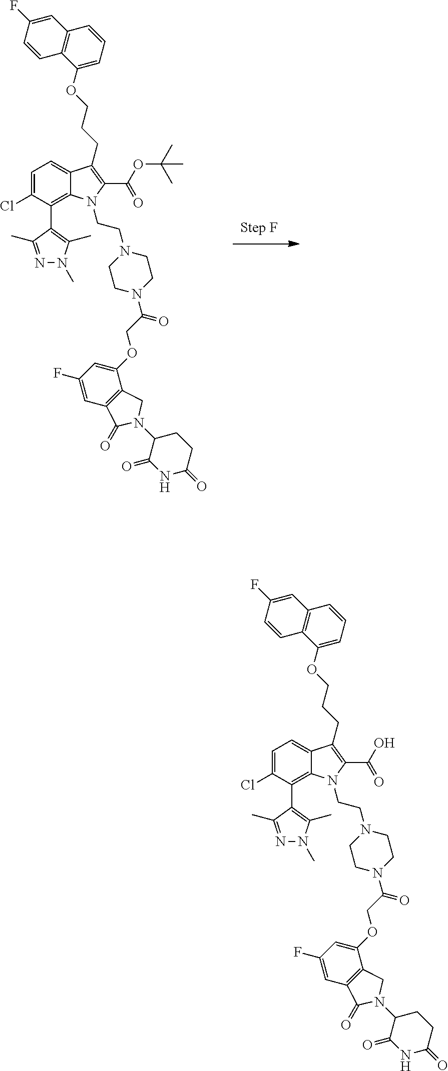 [SMILES: Cc1nn(C)c(C)c1-c1c(Cl)ccc2c(CCCOc3cccc4cc(F)ccc34)c(C(=O)OC(C)(C)C)n(CCN3CCN(C(=O)COc4cc(F)cc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4cc(F)cc5c4CN(C4CCC(=O)NC4=O)C5=O)CC3)c12]

tert-Butyl 6-chloro-1-{2-[4-(2-{[2-(2,6-dioxopiperidin-3-yl)-6-fluoro-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (45.4 mg, crude) was dissolved in dry DCM (0.500 mL) and TFA (0.500 mL, 6.529 mmol) was added. Reaction was stirred for 16 h under argon atmosphere at room temperature. After complete conversion of the starting material, the solution was concentrated to dryness under reduced pressure. The resulting residue was dissolved in DMSO and purified with preparative HPLC (C18, H₂O:MeCN+0.1% FA) to give 6-chloro-1-(2-(4-(2-((2-(2,6-dioxopiperidin-3-yl)-6-fluoro-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (13.2 mg, 0.014 mmol, 37.8% over 2 steps) as white solid.

LCMS (ESI+): m/z 936.2 [M+H]⁺

¹H NMR (500 MHz, DMSO, 353 K) δ 10.66 (s, 1H), 8.24 (dd, J=9.2, 5.8 Hz, 1H), 7.71 (d, J=8.6 Hz, 1H), 7.58 (dd, J=10.4, 2.6 Hz, 1H), 7.46-7.39 (m, 2H), 7.36-7.29 (m, 1H), 7.22 (d, J=8.5 Hz, 1H), 7.10-7.03 (m, 2H), 6.87 (dd, J=5.7, 3.0 Hz, 1H), 5.04 (dd, J=13.1, 5.2 Hz, 1H), 4.93 (s, 2H), 4.37 (d, J=16.5 Hz, 1H), 4.33-4.22 (m, 4H), 4.22-4.15 (m, 1H), 3.76 (d, J=1.4 Hz, 3H), 3.38-3.32 (m, 4H), 3.30-3.26 (m, 2H), 2.87 (ddd, J=17.4, 13.4, 5.5 Hz, 1H), 2.66-2.58 (m, 1H), 2.44 (qd, J=13.0, 4.6 Hz, 1H), 2.28-2.20 (m, 2H), 2.17-2.10 (m, 5H), 2.10-2.02 (m, 2H), 2.01 (s, 3H), 1.89 (s, 3H).

### Example 113. 6-Chloro-1-(2-(4-(2-((2-(2,6-dioxopiperidin-3-yl)-6-fluoro-1-oxoisoindolin-5-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (344)

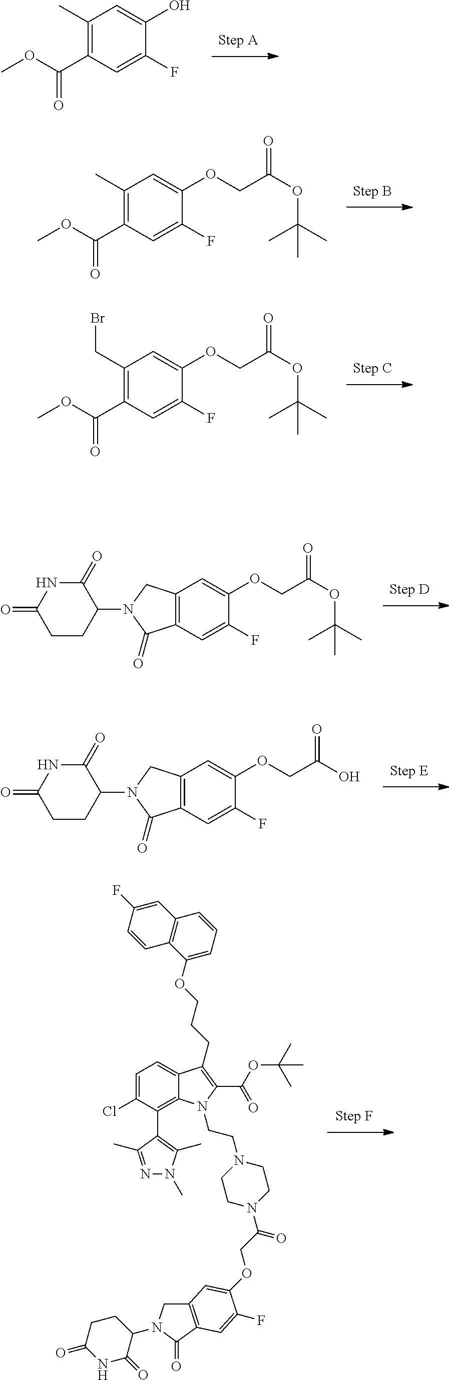

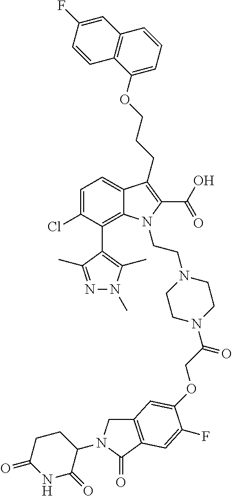 [SMILES: Cc1nn(C)c(C)c1-c1c(Cl)ccc2c(CCCOc3cccc4cc(F)ccc34)c(C(=O)O)n(CCN3CCN(C(=O)COc4cc5c(cc4F)C(=O)N(C4CCC(=O)NC4=O)C5)CC3)c12]

### Step A

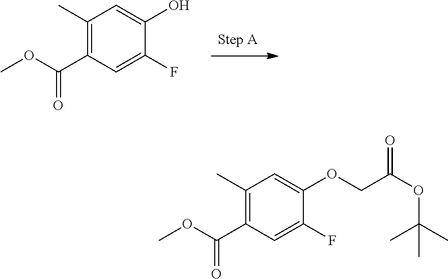 [SMILES: COC(=O)c1cc(F)c(O)cc1C ; COC(=O)c1cc(F)c(OCC(=O)OC(C)(C)C)cc1C]

To a well stirred solution of methyl 5-fluoro-4-hydroxy-2-methylbenzoate (3 g, 16.3 mmol) in DMF (70 mL) were added K₂CO₃ (2.5 g, 18.1 mmol). Tert-butyl bromoacetate (4.8 mL, 24.451 mmol) was then added dropwise and the resulting reaction mixture was allowed to stir at 60° C. for 24 h under nitrogen. After complete consumption of the starting material (monitored by TLC and LCMS), the reaction mixture was diluted with EtOAc and washed successively with cold water followed and brine. The organic layer was dried over anhydrous Na₂SO₄ and evaporated under reduced pressure to get crude which was purified with flash column chromatography (SiO₂, 10% EtOAc in hexane) to get methyl 4-(2-(tert-butoxy)-2-oxoethoxy)-5-fluoro-2-methylbenzoate (3 g, 10.06 mmol, 62%) of as white solid.

### Step B

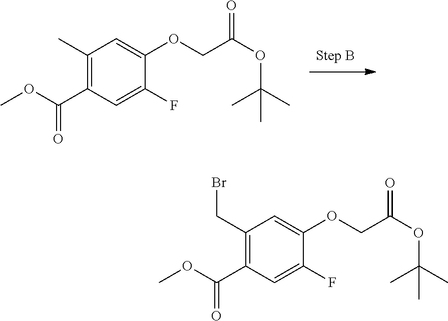

To a solution of methyl 4-(2-(tert-butoxy)-2-oxoethoxy)-5-fluoro-2-methylbenzoate (3 g, 10.06 mmol) in dry CCl₄ (100 mL) were added NBS (2.3 g, 13.07 mmol) followed by AIBN (0.33 g, 2.01 mmol) at 0° C. under nitrogen. The reaction mixture was then allowed to stir at 70° C. for 3 h. After complete consumption of the starting material (monitored by TLC and LCMS), the reaction mixture was diluted with EtOAc and washed successively with water and brine. The organic layer was dried over anhydrous Na₂SO₄ and evaporated under reduced pressure to get 2.5 g of crude methyl 2-(bromomethyl)-4-(2-(tert-butoxy)-2-oxoethoxy)-5-fluorobenzoate which was then used for the next step without further purification

LCMS (ESI+): m/z 321.0 [M−tBu+H]

### Step C

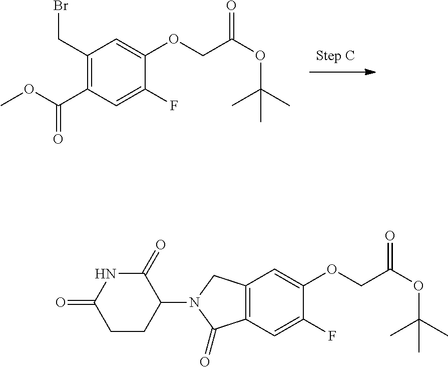 [SMILES: COC(=O)c1cc(F)c(OCC(=O)OC(C)(C)C)cc1CBr ; CC(C)(C)OC(=O)COc1cc2c(cc1F)C(=O)N(C1CCC(=O)NC1=O)C2]

To a solution of methyl 2-(bromomethyl)-4-(2-(tert-butoxy)-2-oxoethoxy)-5-fluorobenzoate (2.5 g, crude) in MeCN (80.0 mL) were added 3-aminopiperidine-2,6-dione hydrochloride (1.53 g, 9.29 mmol) followed by DIPEA (2.7 mL, 19.9 mmol) under nitrogen atmosphere. The reaction mixture was stirred at 70° C. for 16 h. After consumption of the starting material (monitored with LCMS), reaction mixture was concentrated and crude material was purified with flash column chromatography (SiO₂, 50% EtOAc in DCM) to get tert-butyl 2-((2-(2,6-dioxopiperidin-3-yl)-6-fluoro-1-oxoisoindolin-5-yl)oxy)acetate (1.8 g, 4.59 mmol, 45% over 2 steps) as brown solid.

LCMS (ESI+): m/z 393.0 [M+H]⁺

### Step D

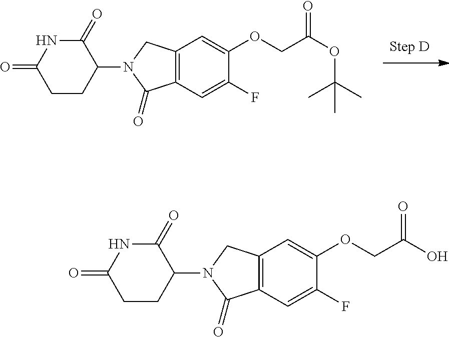 [SMILES: CC(C)(C)OC(=O)COc1cc2c(cc1F)C(=O)N(C1CCC(=O)NC1=O)C2 ; O=C(O)COc1cc2c(cc1F)C(=O)N(C1CCC(=O)NC1=O)C2]

To a solution of tert-butyl 2-((2-(2,6-dioxopiperidin-3-yl)-6-fluoro-1-oxoisoindolin-5-yl)oxy)acetate (1.8 g, 4.59 mmol) in DCM (20 mL) was added TFA (20 mL) at 0° C. dropwise to the reaction mixture and the reaction mixture was allowed to stir at ambient temperature for 16 h under nitrogen. After complete consumption of the starting material (monitored by TLC and LCMS), the volatiles were evaporated under reduced pressure to get the crude compound which was then purified by triturating it with Et₂O and pentane to afford (1.2 g, 3.57 mmol, 78%) of 2-((2-(2,6-dioxopiperidin-3-yl)-6-fluoro-1-oxoisoindolin-5-yl)oxy)acetic acid as white solid.

LCMS (ESI+): m/z 337.15 [M+H]⁺

### Step E

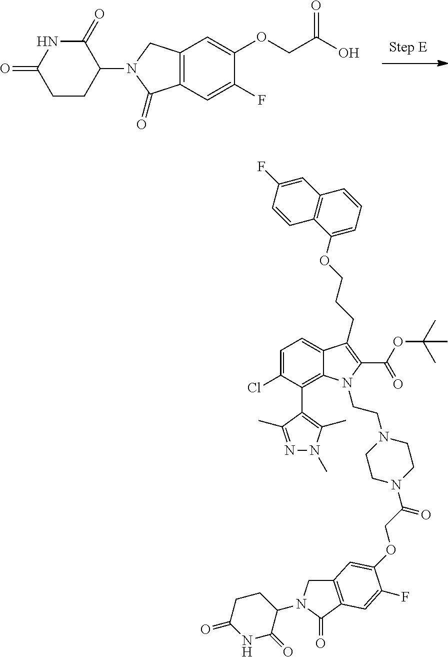 [SMILES: O=C(O)COc1cc2c(cc1F)C(=O)N(C1CCC(=O)NC1=O)C2 ; Cc1nn(C)c(C)c1-c1c(Cl)ccc2c(CCCOc3cccc4cc(F)ccc34)c(C(=O)OC(C)(C)C)n(CCN3CCN(C(=O)COc4cc5c(cc4F)C(=O)N(C4CCC(=O)NC4=O)C5)CC3)c12]

2-{[2-(2,6-Dioxopiperidin-3-yl)-6-fluoro-1-oxo-2,3-dihydro-1H-isoindol-5-yl]oxy}acetic acid (13.7 mg, 0.041 mmol) and tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (25.0 mg, 0.037 mmol) were dissolved in dry DMF (1.0 mL) under argon atmosphere and DIPEA (0.019 mL, 0.111 mmol) was added. To it, HATU (14.8 mg, 0.039 mmol) was added and the reaction (monitored with LCMS), was stirred for 10 min at room temperature under argon. After complete consumption of the starting material, the solution was diluted with DCM and washed with brine and water. The organic layer was dried over anhydrous MgSO₄, filtered and dried under reduced pressure to get crude tert-butyl 6-chloro-1-{2-[4-(2-{[2-(2,6-dioxopiperidin-3-yl)-6-fluoro-1-oxo-2,3-dihydro-1H-isoindol-5-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (41.1 mg) as yellow oil, which was used directly in the next step.

LCMS (ESI+): m/z 992.5 [M+H]⁺

### Step F

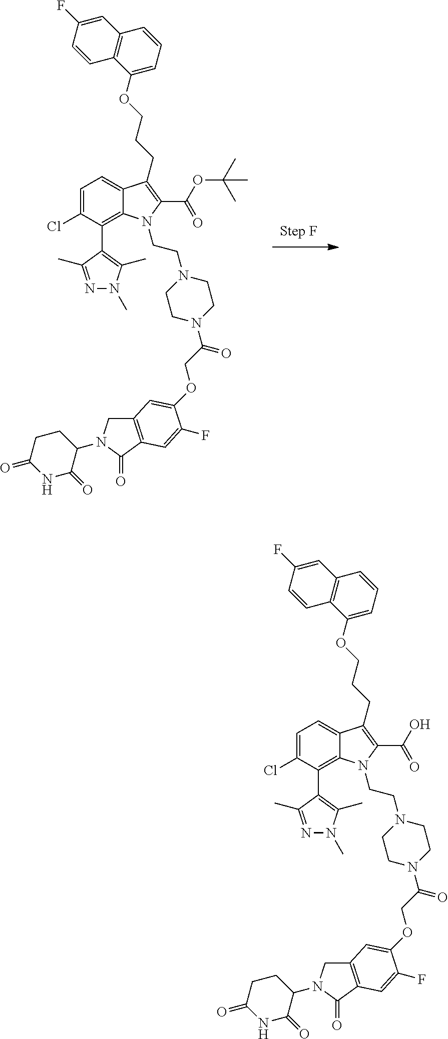 [SMILES: Cc1nn(C)c(C)c1-c1c(Cl)ccc2c(CCCOc3cccc4cc(F)ccc34)c(C(=O)OC(C)(C)C)n(CCN3CCN(C(=O)COc4cc5c(cc4F)C(=O)N(C4CCC(=O)NC4=O)C5)CC3)c12 ; Cc1nn(C)c(C)c1-c1c(Cl)ccc2c(CCCOc3cccc4cc(F)ccc34)c(C(=O)O)n(CCN3CCN(C(=O)COc4cc5c(cc4F)C(=O)N(C4CCC(=O)NC4=O)C5)CC3)c12]

tert-Butyl 6-chloro-1-{2-[4-(2-{[2-(2,6-dioxopiperidin-3-yl)-6-fluoro-1-oxo-2,3-dihydro-1H-isoindol-5-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (41.1 mg, crude) was dissolved under argon in dry DCM (0.400 mL) and TFA (0.400 mL, 5.224 mmol) was added. Reaction (monitored with LCMS) was stirred at room temperature under argon for 16 h. After complete conversion of the starting material, the solution was concentrated to dryness under reduced pressure. The resulting residue was dissolved in DMSO and purified with preparative HPLC (C18, H₂O:MeCN+0.1% FA) to get 6-chloro-1-(2-(4-(2-((2-(2,6-dioxopiperidin-3-yl)-6-fluoro-1-oxoisoindolin-5-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (16.2 mg, 0.017 mmol, 46% over 2 steps).

LCMS (ESI+): m/z 936.15 [M+H]⁺

¹H NMR (500 MHz, DMSO, 353 K) δ 10.64 (s, 1H), 8.24 (dd, J=9.2, 5.9 Hz, 1H), 7.71 (d, J=8.5 Hz, 1H), 7.58 (dd, J=10.4, 2.6 Hz, 1H), 7.47 (d, J=9.9 Hz, 1H), 7.44-7.38 (m, 2H), 7.32 (ddd, J=9.3, 8.6, 2.6 Hz, 1H), 7.29 (d, J=7.2 Hz, 1H), 7.22 (d, J=8.5 Hz, 1H), 6.87 (dd, J=5.7, 2.9 Hz, 1H), 5.01 (dd, J=13.1, 5.2 Hz, 1H), 4.93 (s, 2H), 4.38 (d, J=17.2 Hz, 1H), 4.34-4.22 (m, 4H), 4.22-4.15 (m, 1H), 3.76 (s, 3H), 3.41-3.32 (m, 4H), 3.32-3.25 (m, 2H), 2.87 (ddd, J=17.4, 13.4, 5.5 Hz, 1H), 2.66-2.60 (m, 1H), 2.38 (qd, J=13.1, 4.6 Hz, 1H), 2.28-2.20 (m, 2H), 2.16-2.12 (m, 4H), 2.12-2.02 (m, 3H), 2.01 (s, 3H), 1.89 (s, 3H).

### Example 114. 6-Chloro-1-{2-[4-(2-{[5-chloro-2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (345)

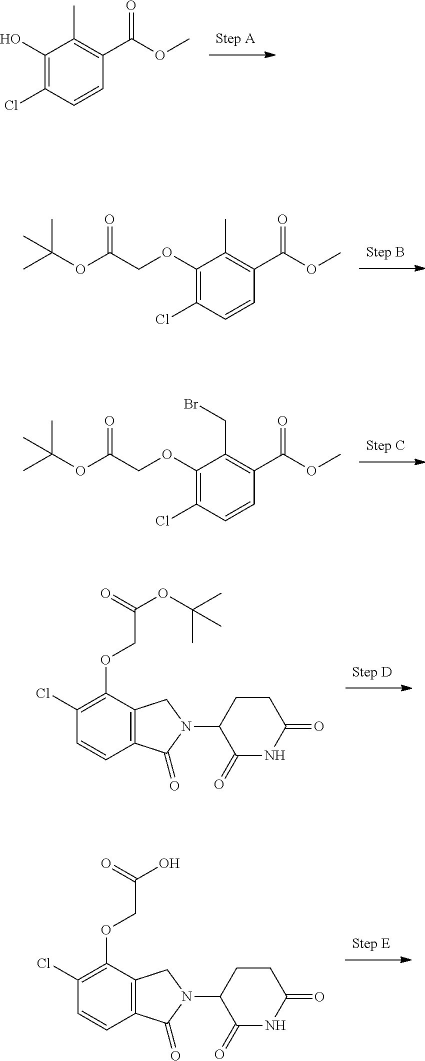

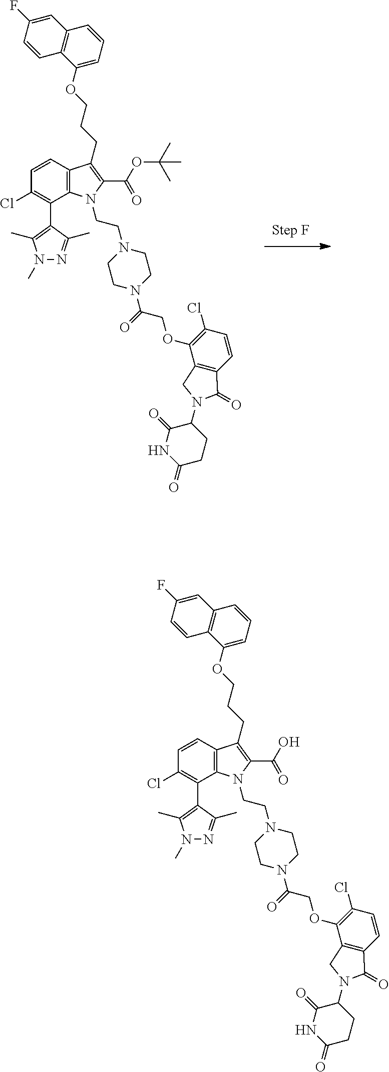 [SMILES: Cc1nn(C)c(C)c1-c1c(Cl)ccc2c(CCCOc3cccc4cc(F)ccc34)c(C(=O)OC(C)(C)C)n(CCN3CCN(C(=O)COc4c(Cl)ccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4c(Cl)ccc5c4CN(C4CCC(=O)NC4=O)C5=O)CC3)c12]

### Step A

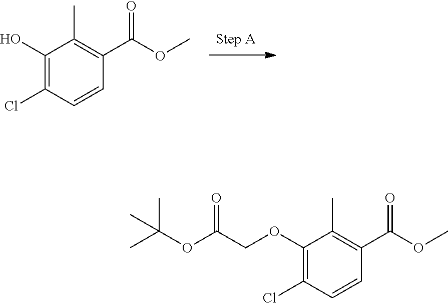 [SMILES: COC(=O)c1ccc(Cl)c(O)c1C ; COC(=O)c1ccc(Cl)c(OCC(=O)OC(C)(C)C)c1C]

To a well stirred solution of methyl 4-chloro-3-hydroxy-2-methylbenzoate (2 g, 10 mmol) in DMF (40 mL) was added KI (0.83 g, 5 mmol), KHCO₃ (2 g, 20 mmol) and tert-butyl bromo acetate (2.2 mL, 15 mmol) successively at room temperature under nitrogen. The reaction mixture was allowed to stir at 70° C. for 16 h. After complete consumption of the starting material (monitored by TLC and LCMS) the reaction mixture was diluted with EtOAc, washed successively with water and brine, the organic layer was dried over Na₂SO₄ and evaporated under reduced pressure. The crude was purified by column chromatography (SiO₂, 40% EtOAc in DCM) to get methyl 3-(2-(tert-butoxy)-2-oxoethoxy)-4-chloro-2-methylbenzoate (2 g, 6.36 mmol, 63%) of as off white solid.

### Step B

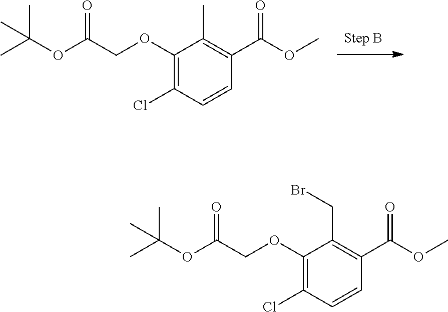

To a stirred solution of methyl 3-(2-(tert-butoxy)-2-oxoethoxy)-4-chloro-2-methylbenzoate (1.7 g, 5.41 mmol) in carbon tetrachloride (20 mL) were added NBS (0.97 g, 5.41 mmol) and AIBN (0.18 g, 1.08 mmol) successively at room temperature under nitrogen. The mixture was allowed to stir under reflux for 16 h. After complete consumption of the starting material (monitored by crude NMR) the reaction mixture was diluted with EtOAc, washed successively with water and brine, the organic layer was dried Na₂SO₄ and evaporated under reduced pressure. The crude was then purified by column chromatography (SiO₂, 5-10% EtOAc in hexane) to get methyl 2-(bromomethyl)-3-(2-(tert-butoxy)-2-oxoethoxy)-4-chlorobenzoate (1.3 g, 3.3 mmol, 61%) of as colorless dense liquid.

### Step C

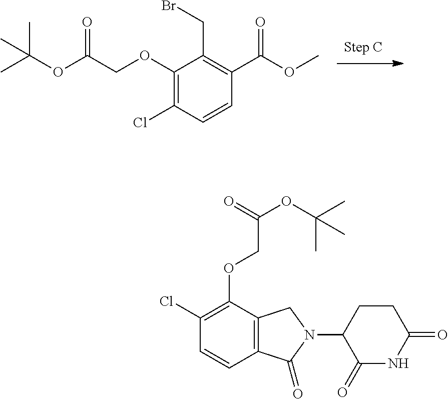 [SMILES: COC(=O)c1ccc(Cl)c(OCC(=O)OC(C)(C)C)c1CBr ; CC(C)(C)OC(=O)COc1c(Cl)ccc2c1CN(C1CCC(=O)NC1=O)C2=O]

To a stirred solution of methyl 2-(bromomethyl)-3-(2-(tert-butoxy)-2-oxoethoxy)-4-chlorobenzoate (1 g, 2.45 mmol) in MeCN (15 mL) was added 3-aminopiperidine-2,6-dione hydrochloride (0.55 g, 3.3 mmol) and DIPEA (1.4 mL, 7.7 mmol) successively under nitrogen at RT. It was then allowed to stir under reflux for 16 h under nitrogen. After complete consumption of the starting material (monitored by TLC and LCMS) the resulting reaction mixture was evaporated under reduced pressure to get the crude compound which was then washed with water and Et₂O successively through a sinter funnel to afford tert-butyl 2-((5-chloro-2-(2,6-dioxopiperidin-3-yl)-1-oxoisoindolin-4-yl)oxy)acetate (600 mg, 1.47 mmol, 57%) of as grey solid.

LCMS (ESI+): m/z 409.0 [M+H]⁺

### Step D

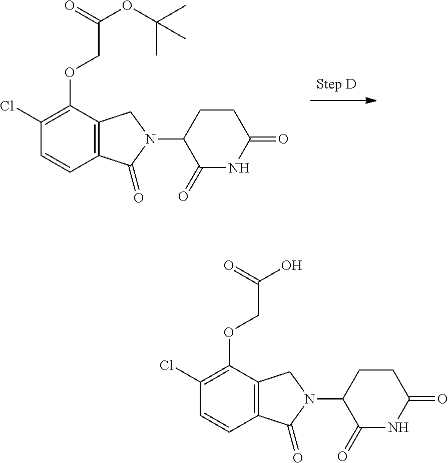 [SMILES: CC(C)(C)OC(=O)COc1c(Cl)ccc2c1CN(C1CCC(=O)NC1=O)C2=O ; O=C(O)COc1c(Cl)ccc2c1CN(C1CCC(=O)NC1=O)C2=O]

To a stirred solution of tert-butyl 2-((5-chloro-2-(2,6-dioxopiperidin-3-yl)-1-oxoisoindolin-4-yl)oxy)acetate (0.6 g, 1.47 mmol) in DCM (18 mL) was added TFA (6 mL) drop wise at 0° C. under nitrogen and the reaction mixture was allowed to stir at room temperature for 3 h. After consuming of starting material (monitored by TLC and LCMS) the volatiles were evaporated under reduced pressure to get the crude compound which was then purified by triturating by Et₂O and pentane to afford 2-((5-chloro-2-(2,6-dioxopiperidin-3-yl)-1-oxoisoindolin-4-yl)oxy)acetic acid (0.38 g, 1.09, 74%) of as off white solid.

LCMS (ESI+): m/z 353.1 [M+H]⁺

### Step E

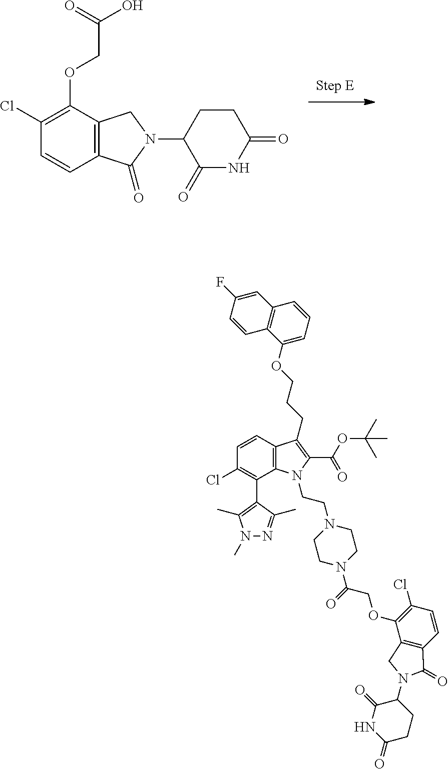 [SMILES: O=C(O)COc1c(Cl)ccc2c1CN(C1CCC(=O)NC1=O)C2=O ; Cc1nn(C)c(C)c1-c1c(Cl)ccc2c(CCCOc3cccc4cc(F)ccc34)c(C(=O)OC(C)(C)C)n(CCN3CCN(C(=O)COc4c(Cl)ccc5c4CN(C4CCC(=O)NC4=O)C5=O)CC3)c12]

To a solution of tert-butyl 6-chloro-3-{3-[(6-fluoronaphthalen-1-yl)oxy]propyl}-1-(2-{octahydropyrrolo[2,3-c]pyrrol-1-yl}ethyl)-7-(1,3,5-trimethyl-1H-pyrazol-4-yl)-1H-indole-2-carboxylate hydrochloride (30.0 mg, 0.044 mmol), 2-((5-chloro-2-(2,6-dioxopiperidin-3-yl)-1-oxoisoindolin-4-yl)oxy)acetic acid (18.8 mg, 0.053 mmol) and HATU (33.8 mg, 0.089 mmo) in dry DMF (2.0 mL) was added DIPEA (0.023 mL, 0.133 mmol). The mixture was stirred at room temperature for 30 min. The crude was diluted with DCM and washed with brine. The organic layer was dried over Na₂SO₄, filtered and concentrated in vacuo to get 52 mg of crude tert-butyl 6-chloro-1-{2-[4-(2-{[5-chloro-2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate as yellow oil which was used in the next step without further purification.

LCMS (ESI+): m/z 1008.25 [M+H]⁺

### Step F

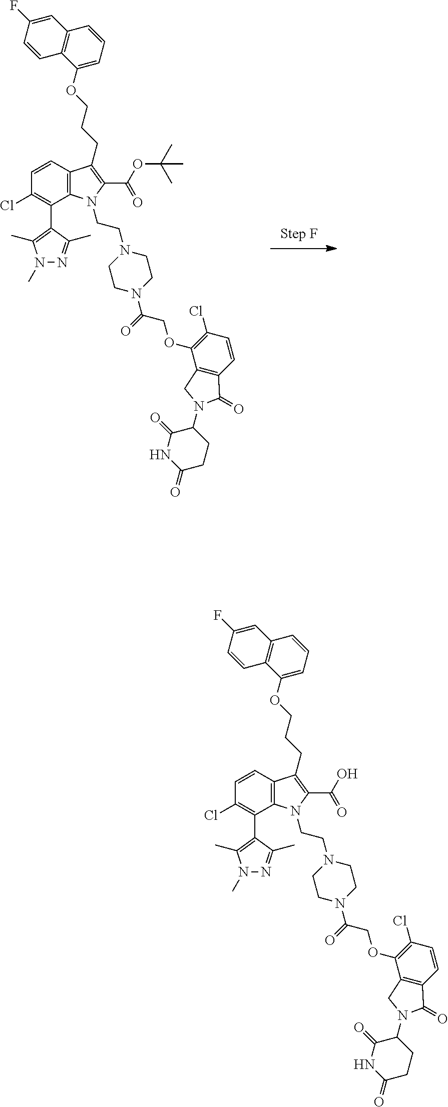 [SMILES: Cc1nn(C)c(C)c1-c1c(Cl)ccc2c(CCCOc3cccc4cc(F)ccc34)c(C(=O)OC(C)(C)C)n(CCN3CCN(C(=O)COc4c(Cl)ccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4c(Cl)ccc5c4CN(C4CCC(=O)NC4=O)C5=O)CC3)c12]

To a solution of tert-butyl 6-chloro-1-{2-[4-(2-{[5-chloro-2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (52 mg, crude) in DCM (1.0 mL) was added TFA (1.0 mL, 13.059 mmol). The mixture was stirred for 18 h at RT. The crude was concentrated in vacuo and purified by preparative HPLC (C18, H₂O:MeCN+0.1% FA) to obtain 6-chloro-1-{2-[4-(2-{[5-chloro-2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (22.0 mg, 0.023 mmol, yield over two steps: 52%) as a white solid.

LCMS (ESI+): m/z 952.2 [M+H]⁺

¹H NMR (500 MHz, DMSO) δ 10.68 (s, 1H), 8.24 (dd, J=9.2, 5.9 Hz, 1H), 7.71 (d, J=8.5 Hz, 1H), 7.63-7.54 (m, 2H), 7.47-7.38 (m, 3H), 7.32 (td, J=8.9, 2.6 Hz, 1H), 7.22 (d, J=8.5 Hz, 1H), 6.87 (dd, J=5.8, 2.9 Hz, 1H), 5.04 (dd, J=13.0, 5.2 Hz, 1H), 4.95-4.87 (m, 2H), 4.61 (d, J=17.2 Hz, 1H), 4.52 (dd, J=17.2, 2.7 Hz, 1H), 4.31-4.22 (m, 3H), 4.22-4.14 (m, 1H), 3.76 (s, 3H), 3.39-3.32 (m, 4H), 3.31-3.26 (m, 2H), 2.88 (ddd, J=17.4, 13.4, 5.5 Hz, 1H), 2.67-2.61 (m, 1H), 2.46-2.35 (m, 1H), 2.27-2.21 (m, 2H), 2.15-2.03 (m, 7H), 2.01 (s, 3H), 1.88 (s, 3H).

### Example 115. 1-(2-(4-(2-(7-Bromo-4-((2,6-dioxopiperidin-3-yl)carbamoyl)-2-methyl-1H-benzo[d]imidazol-1-yl)acetyl)piperazin-1-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylic acid (269)

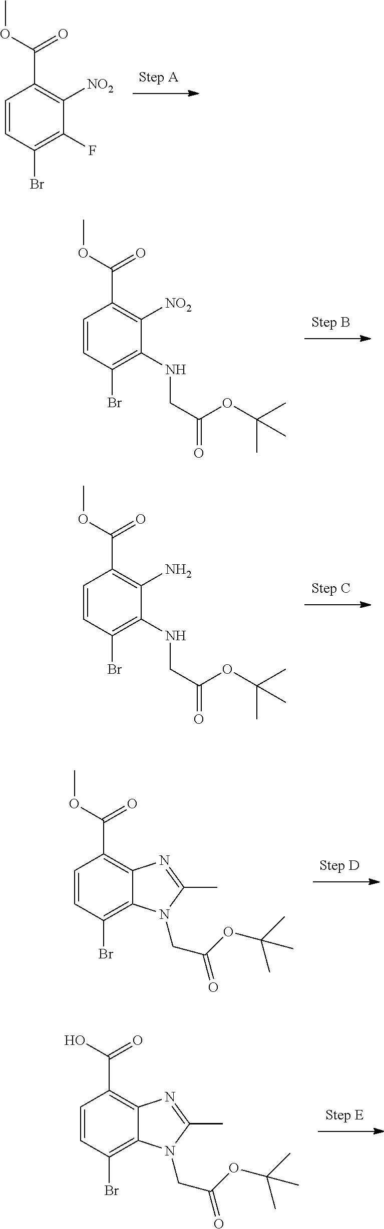 [SMILES: COC(=O)c1ccc(Br)c(F)c1[N+](=O)[O-] ; COC(=O)c1ccc(Br)c(NCC(=O)OC(C)(C)C)c1[N+](=O)[O-] ; COC(=O)c1ccc(Br)c(NCC(=O)OC(C)(C)C)c1N ; COC(=O)c1ccc(Br)c2c1nc(C)n2CC(=O)OC(C)(C)C ; Cc1nc2c(C(=O)O)ccc(Br)c2n1CC(=O)OC(C)(C)C]

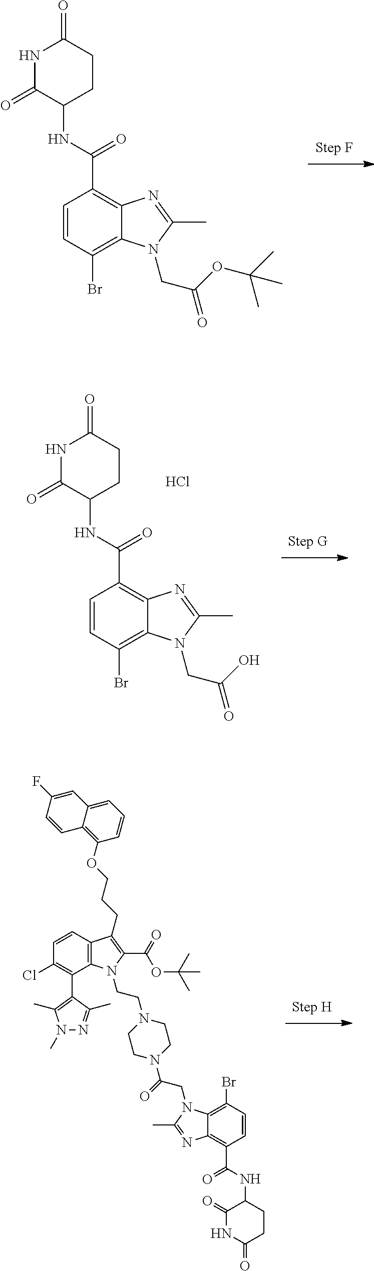

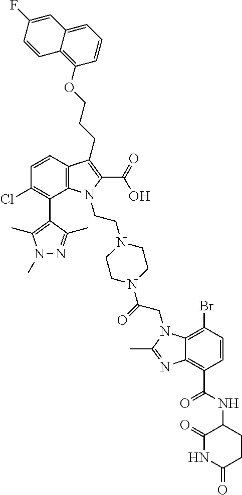 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)ccc(Br)c54)CC3)c12]

### Step A

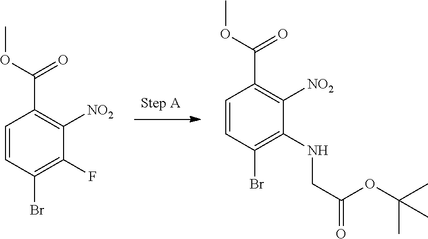 [SMILES: COC(=O)c1ccc(Br)c(F)c1[N+](=O)[O-] ; COC(=O)c1ccc(Br)c(NCC(=O)OC(C)(C)C)c1[N+](=O)[O-]]

To a solution of methyl 4-bromo-3-fluoro-2-nitrobenzoate (10.3 g, 37.0 mmol) in DMSO (120 mL) were added tert-butyl glycinate (10.1 mL, 74.1 mmol) and DIPEA (18.5 mL, 111.1 mmol). The reaction mixture was then stirred for 2 h at 70° C. After complete consumption of the starting, the reaction mixture was cooled to room temperature, diluted with EtOAc and washed successively by water and brine. The organic layer was dried over anhydrous Na₂SO₄, concentrated and purified by column chromatography (SiO₂, 20% EtOAc in hexane) to afford methyl 4-bromo-3-((2-(tert-butoxy)-2-oxoethyl)amino)-2-nitrobenzoate (7.5 g, 19.3 mmol, 52%) as yellow solid.

LCMS (ESI+): m/z 389.0 [M+H]⁺

### Step B

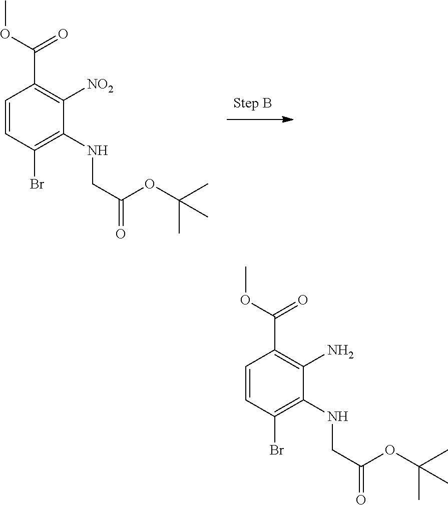 [SMILES: COC(=O)c1ccc(Br)c(NCC(=O)OC(C)(C)C)c1[N+](=O)[O-] ; COC(=O)c1ccc(Br)c(NCC(=O)OC(C)(C)C)c1N]

To a stirring solution of methyl 4-bromo-3-((2-(tert-butoxy)-2-oxoethyl)amino)-2-nitrobenzoate (3.2 g, 8.2 mmol) in EtOAc (100 mL) were added platinum sulfided on carbon (1.6 g). The reaction mixture was then stirred for 2 h at room temperature under hydrogen atmosphere. After complete consumption of the starting material, the reaction mixture was filtered through Celite® pad and washed with EtOAc. The filtrate was concentrated under reduced pressure to get the crude methyl 2-amino-4-bromo-3-((2-(tert-butoxy)-2-oxoethyl)amino)benzoate which was used for the next step without further purification.

### Step C

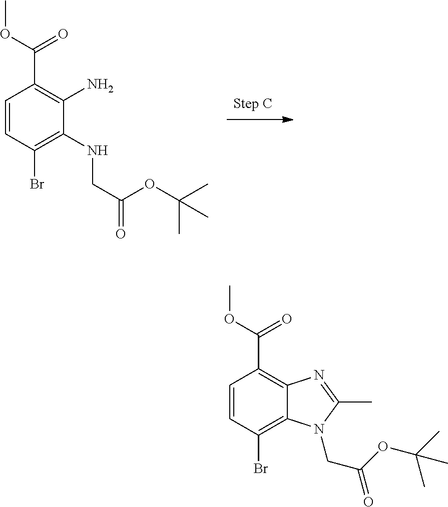 [SMILES: COC(=O)c1ccc(Br)c(NCC(=O)OC(C)(C)C)c1N ; COC(=O)c1ccc(Br)c2c1nc(C)n2CC(=O)OC(C)(C)C]

To a solution of methyl 2-amino-4-bromo-3-((2-(tert-butoxy)-2-oxoethyl)amino)benzoate (3.2 g, crude) in HFIP (40 mL) was added 1,1,1-triethoxyethane (6.525 g, 40.2 mmol) and the reaction mixture was then stirred for 16 h at RT. After complete consumption of the starting material, the volatiles were evaporated under reduced pressure to get the crude mixture which was then purified by column chromatography (SiO₂, 80% EtOAc in hexane) to afford methyl 7-bromo-1-(2-(tert-butoxy)-2-oxoethyl)-2-methyl-1H-benzo[d]imidazole-4-carboxylate (1.0 g, 2.6 mmol, 32% over 2 steps) as off white solid.

LCMS (ESI+): m/z 383.1 & 385.1 [M+H]⁺

### Step D

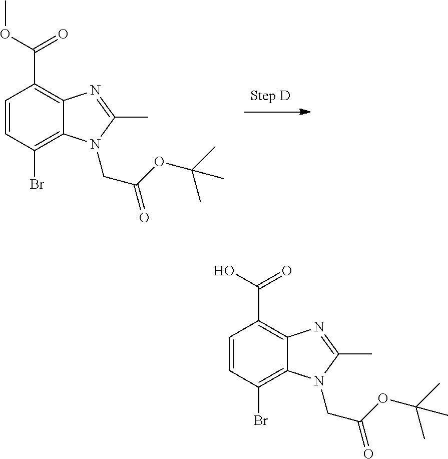 [SMILES: COC(=O)c1ccc(Br)c2c1nc(C)n2CC(=O)OC(C)(C)C ; Cc1nc2c(C(=O)O)ccc(Br)c2n1CC(=O)OC(C)(C)C]

Methyl 7-bromo-1-(2-(tert-butoxy)-2-oxoethyl)-2-methyl-1H-benzo[d]imidazole-4-carboxylate (75.0 mg, 0.196 mmol) was dissolved in dry MeCN (3.9 mL) and LiBr (339.9 mg, 3.914 mmol) was added, followed by Et₃N (0.327 mL, 2.348 mmol). Reaction was stirred at room temperature for 72 h. After complete consumption of the starting material, the mixture was concentrated under reduced pressure and purified with reverse phase flash chromatography (C18, H₂O:MeCN+0.1% FA) to give 7-bromo-1-[2-(tert-butoxy)-2-oxoethyl]-2-methyl-1H-1,3-benzodiazole-4-carboxylic acid as white solid.

LCMS (ESI+): m/z 369.0 & 370.9 [M+H]⁺

### Step E

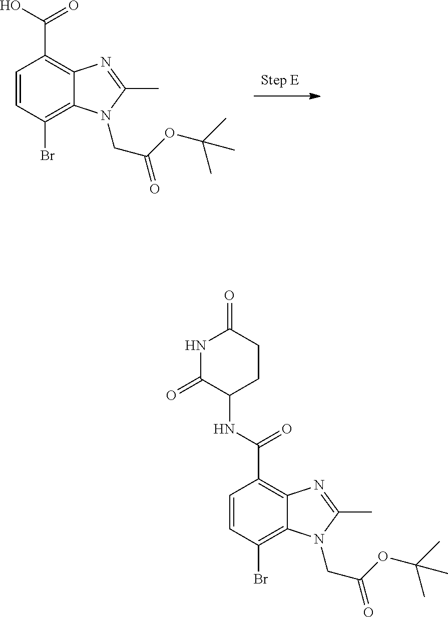 [SMILES: Cc1nc2c(C(=O)O)ccc(Br)c2n1CC(=O)OC(C)(C)C ; Cc1nc2c(C(=O)NC3CCC(=O)NC3=O)ccc(Br)c2n1CC(=O)OC(C)(C)C]

7-Bromo-1-[2-(tert-butoxy)-2-oxoethyl]-2-methyl-1H-1,3-benzodiazole-4-carboxylic acid obtained in the previous step was dissolved in dry DMF (2.0 mL) under argon atmosphere and DIPEA (0.104 mL, 0.595 mmol) was added, followed by HATU (90.5 mg, 0.238 mmol) and 3-aminopiperidine-2,6-dione hydrochloride (49.0 mg, 0.298 mmol). Reaction was stirred for 10 min at room temperature. After complete consumption of the starting material, the solvent was evaporated under reduced pressure, resulting residue dissolved in DMSO and purified with reverse phase flash chromatography (C18, H₂O:MeCN+0.1% FA) to give tert-butyl 2-{7-bromo-4-[(2,6-dioxopiperidin-3-yl)carbamoyl]-2-methyl-1H-1,3-benzodiazol-1-yl}acetate (54.7 mg, 0.114 mmol, 58% over 2 steps) as white solid.

LCMS (ESI+): m/z 479.0 & 480.95 [M+H]⁺

### Step F

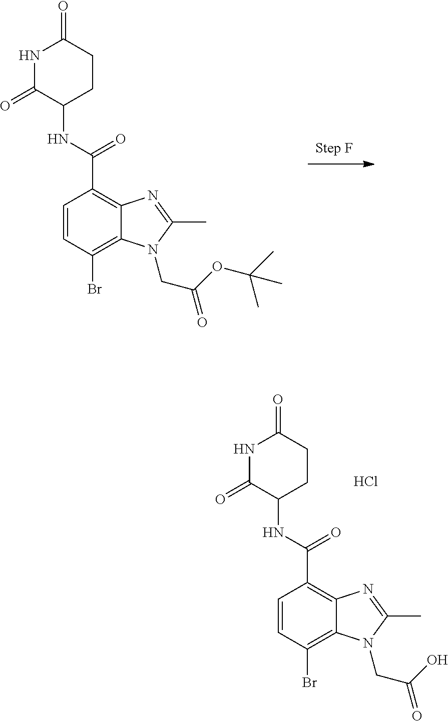 [SMILES: Cc1nc2c(C(=O)NC3CCC(=O)NC3=O)ccc(Br)c2n1CC(=O)OC(C)(C)C ; Cc1nc2c(C(=O)NC3CCC(=O)NC3=O)ccc(Br)c2n1CC(=O)O ; Cl]

tert-Butyl 2-{7-bromo-4-[(2,6-dioxopiperidin-3-yl)carbamoyl]-2-methyl-1H-1,3-benzodiazol-1-yl}acetate (20.0 mg, 0.042 mmol) was dissolved in dry DCM (0.300 mL) under argon atmosphere and TFA (0.320 mL, 4.172 mmol) was added. Reaction, was stirred for 16 h at room temperature. After complete consumption of the starting material, solvent along with TFA were evaporated under reduced pressure and an 1M aqueous solution of HCl was added. The solution was concentrated to dryness and the procedure was repeated twice. The resulting residue was dissolved in water and freeze dried to give 2-{7-bromo-4-[(2,6-dioxopiperidin-3-yl)carbamoyl]-2-methyl-1H-1,3-benzodiazol-1-yl}acetic acid hydrochloride (19.1 mg, 0.042 mmol, 99.6%) as yellowish solid.

LCMS (ESI+): m/z 422.9 [M+H]⁺

### Step G

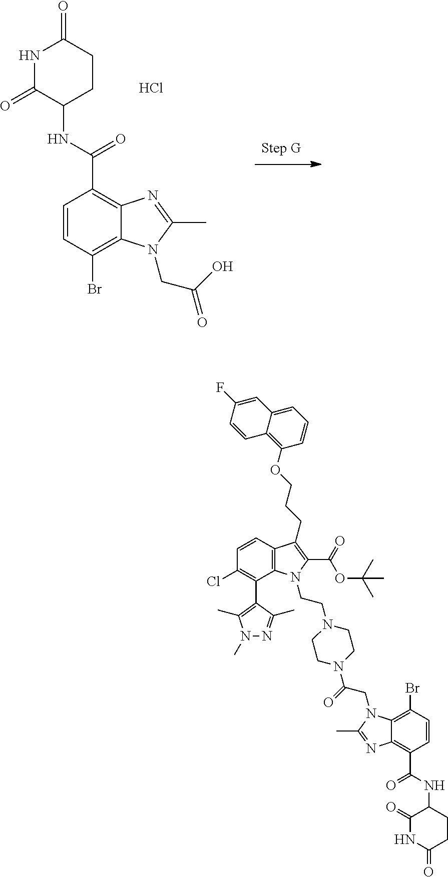 [SMILES: Cc1nc2c(C(=O)NC3CCC(=O)NC3=O)ccc(Br)c2n1CC(=O)O ; Cl ; Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)ccc(Br)c54)CC3)c12]

2-{7-Bromo-4-[(2,6-dioxopiperidin-3-yl)carbamoyl]-2-methyl-1H-1,3-benzodiazol-1-yl}acetic acid hydrochloride (18.0 mg, 0.039 mmol) was dissolved in dry DMF (1.4 mL) under argon atmosphere and DIPEA (0.019 mL, 0.107 mmol), followed by tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (24.0 mg, 0.036 mmol) was added. To it, HATU (14.2 mg, 0.037 mmol) was added dropwise as a solution in DMF (0.6 mL) and the reaction was stirred for 30 min. After complete consumption of the starting material, the reaction mixture was diluted with DCM and poured into brine. The aqueous layer was extracted with DCM and the combined organics were washed with aqueous saturated NaHCO₃, again brine and water. Organic layer was dried over anhydrous Na₂SO₄, filtrated and dried under reduced pressure. Crude tert-butyl 1-{2-[4-(2-{7-bromo-4-[(2,6-dioxopiperidin-3-yl)carbamoyl]-2-methyl-1H-1,3-benzodiazol-1-yl}acetyl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylate (30.1 mg) was used in the next step without further purification.

LCMS (ESI+): m/z 1080.15 [M+H]⁺

### Step H

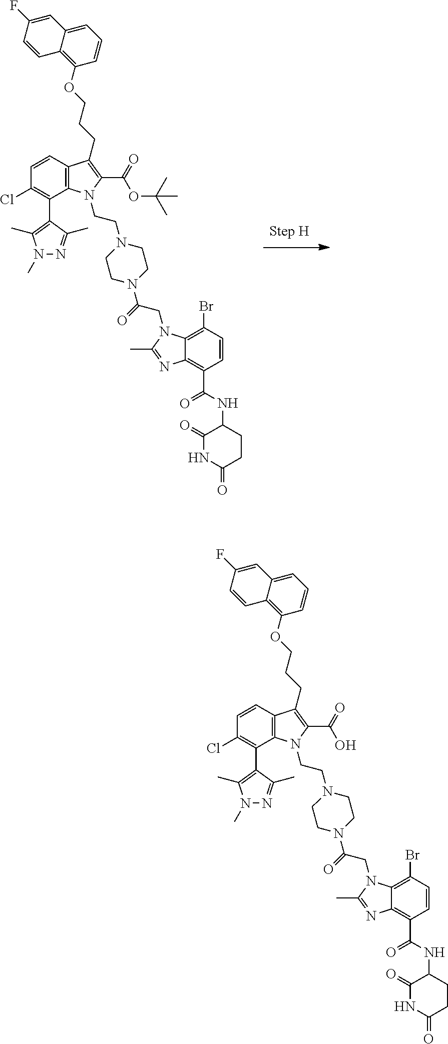 [SMILES: Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)ccc(Br)c54)CC3)c12 ; Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)ccc(Br)c54)CC3)c12]

Crude tert-butyl 1-{2-[4-(2-{7-bromo-4-[(2,6-dioxopiperidin-3-yl)carbamoyl]-2-methyl-1H-1,3-benzodiazol-1-yl}acetyl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylate (30.1 mg) was dissolved in dry DCM (0.201 mL) under argon atmosphere and TFA (0.214 mL, 2.788 mmol) was added. Reaction was stirred AT room temperature for 16 h. After complete consumption of the starting material, the reaction mixture was concentrated under reduced pressure. The resulting residue was dissolved in DMSO, passed through a syringe filter and the reaction product was purified with preparative HPLC (C18, H₂O:MeCN+0.1% FA) to give 1-{2-[4-(2-{7-bromo-4-[(2,6-dioxopiperidin-3-yl)carbamoyl]-2-methyl-1H-1,3-benzodiazol-1-yl}acetyl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylic acid (21.7 mg, 0.021 mmol, 58% over 2 steps) as white solid.

LCMS (ESI+): m/z 1024.1 [M+H]⁺

¹H NMR (500 MHz, DMSO, 353 K) δ 10.61 (s, 1H), 10.18 (d, J=7.1 Hz, 1H), 8.27 (dd, J=9.2, 5.9 Hz, 1H), 7.78 (d, J=8.2 Hz, 1H), 7.75 (d, J=8.5 Hz, 1H), 7.61 (dd, J=10.4, 2.6 Hz, 1H), 7.48 (d, J=8.2 Hz, 1H), 7.47-7.42 (m, 2H), 7.34 (td, J=8.9, 2.6 Hz, 1H), 7.25 (d, J=8.5 Hz, 1H), 6.90 (dd, J=5.3, 3.3 Hz, 1H), 5.51-5.41 (m, 2H), 4.87 (ddd, J=12.3, 7.1, 5.2 Hz, 1H), 4.38-4.20 (m, 4H), 3.80 (s, 3H), 3.54-3.40 (m, 4H), 3.32 (dd, J=8.5, 6.5 Hz, 2H), 2.83 (ddd, J=17.5, 13.1, 5.5 Hz, 1H), 2.66-2.60 (m, 1H), 2.54 (s, 3H), 2.39-2.01 (m, 13H), 1.93 (s, 3H).

### Example 116. 6-Chloro-1-{2-[4-(2-{4-[(2,6-dioxopiperidin-3-yl)carbamoyl]-2,7-dimethyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (270)

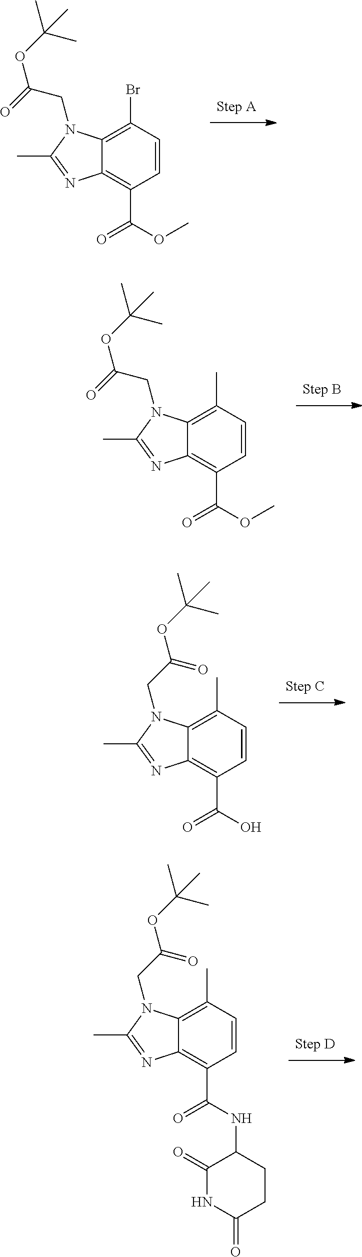

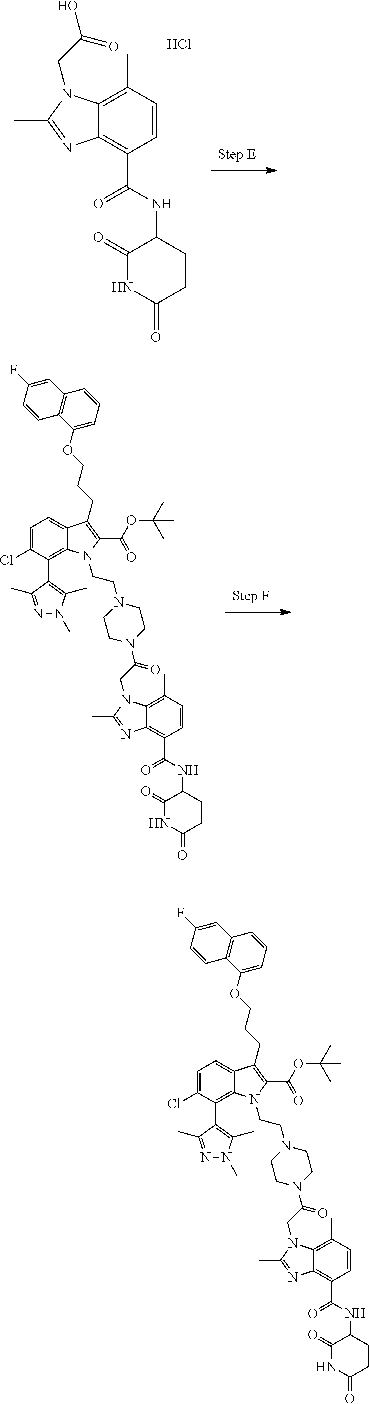 [SMILES: Cc1ccc(C(=O)NC2CCC(=O)NC2=O)c2nc(C)n(CC(=O)O)c12 ; Cl ; Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)ccc(C)c54)CC3)c12 ; Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)ccc(C)c54)CC3)c12]

### Step A

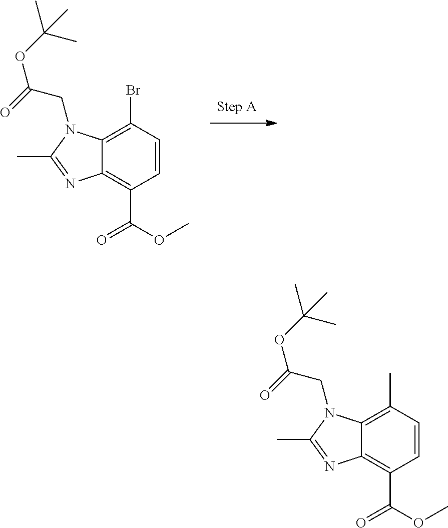 [SMILES: COC(=O)c1ccc(Br)c2c1nc(C)n2CC(=O)OC(C)(C)C ; COC(=O)c1ccc(C)c2c1nc(C)n2CC(=O)OC(C)(C)C]

To a solution of methyl 7-bromo-1-(2-(tert-butoxy)-2-oxoethyl)-2-methyl-1H-benzo[d]imidazole-4-carboxylate (2.2 g, 5.7 mmol) in dioxane (40 mL), were added methyl boronic acid (1.19 g, 20.1 mmol) followed by K₃PO₄ (3.6 g, 17.2 mmol). The reaction mixture was then deoxygenated by argon gas for 10 min. S—PHOS (0.7 g, 1.7 mmol) and Pd(OAc)₂ (193 mg, 0.86 mmol) were added successively to the reaction mixture under argon atmosphere and the reaction mixture was allowed to stir at 90° C. for 16 h. After complete consumption of the starting material the reaction was cooled to room temperature, filtered through Celite® pad and concentrated under reduced pressure to get the crude material. The residue was purified by column chromatography (SiO₂, 80% EtOAc in hexane) to get methyl 1-[2-(tert-butoxy)-2-oxoethyl]-2,7-dimethyl-1H-1,3-benzodiazole-4-carboxylate (950 mg, 2.98 mmol, 52%) as off white solid.

LCMS (ESI+): m/z 319.2 [M+H]⁺

### Step B

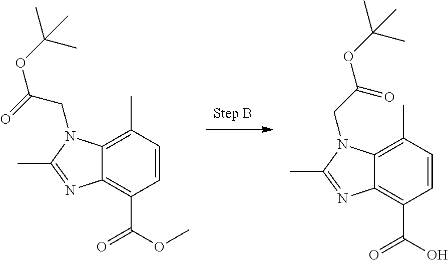 [SMILES: COC(=O)c1ccc(C)c2c1nc(C)n2CC(=O)OC(C)(C)C ; Cc1ccc(C(=O)O)c2nc(C)n(CC(=O)OC(C)(C)C)c12]

Methyl 1-[2-(tert-butoxy)-2-oxoethyl]-2,7-dimethyl-1H-1,3-benzodiazole-4-carboxylate (105.0 mg, 0.330 mmol) was dissolved in MeCN (5.0 mL) and Et₃N (0.459 mL, 3.298 mmol) was added. To this mixture LiBr (572.8 mg, 6.596 mmol) dissolved in water (1.0 mL) was added and mixture was stirred in room temperature for 5 days. The mixture was purified by reverse phase flash chromatography (C18, H₂O:MeCN+0.1% FA) to give a corresponding 1-[2-(tert-butoxy)-2-oxoethyl]-2,7-dimethyl-1H-1,3-benzodiazole-4-carboxylic acid (98.0 mg, 0.322 mmol, 97.6%) as off white solid.

LCMS (ESI+): m/z 619.1 [M+H]⁺

### Step C

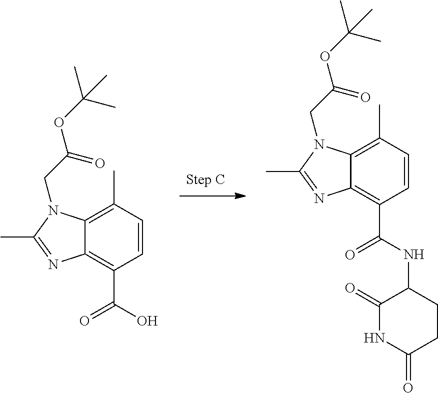 [SMILES: Cc1ccc(C(=O)O)c2nc(C)n(CC(=O)OC(C)(C)C)c12 ; Cc1ccc(C(=O)NC2CCC(=O)NC2=O)c2nc(C)n(CC(=O)OC(C)(C)C)c12]

To a solution of 1-[2-(tert-butoxy)-2-oxoethyl]-2,7-dimethyl-1H-1,3-benzodiazole-4-carboxylic acid (27.0 mg, 0.089 mmol), 3-aminopiperidine-2,6-dione hydrochloride (17.5 mg, 0.106 mmol) and HATU (67.5 mg, 0.177 mmol) in dry DMF (1.1 mL) was added DIPEA (0.046 mL, 0.266 mmol). The mixture was stirred at room temperature for 18 h. The crude was purified by reverse phase flash chromatography (C18, H₂O:MeCN+0.1% FA) to obtain tert-butyl 2-{4-[(2,6-dioxopiperidin-3-yl)carbamoyl]-2,7-dimethyl-1H-1,3-benzodiazol-1-yl}acetate (24.0 mg, 0.042 mmol, 47.0%) as a white solid.

LCMS (ESI+): m/z 415.1 [M+H]⁺

### Step D

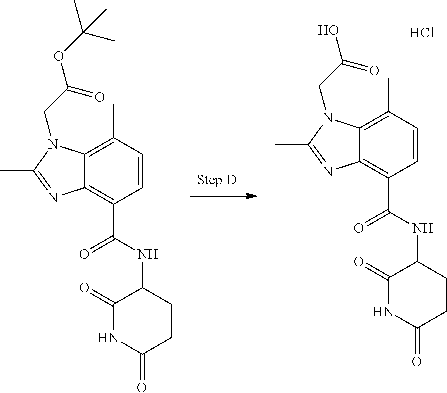 [SMILES: Cc1ccc(C(=O)NC2CCC(=O)NC2=O)c2nc(C)n(CC(=O)OC(C)(C)C)c12 ; Cc1ccc(C(=O)NC2CCC(=O)NC2=O)c2nc(C)n(CC(=O)O)c12 ; Cl]

To a solution of tert-butyl 2-{4-[(2,6-dioxopiperidin-3-yl)carbamoyl]-2,7-dimethyl-1H-1,3-benzodiazol-1-yl}acetate (65.0 mg, 0.157 mmol) in DCM (1.0 mL) was added TFA (1.0 mL, 13.059 mmol). The mixture was stirred for 18 h at RT. The crude was concentrated in vacuo and dissolved in H₂O. To the solution was added HCl and it was evaporated. The product, 2-{4-[(2,6-dioxopiperidin-3-yl)carbamoyl]-2,7-dimethyl-1H-1,3-benzodiazol-1-yl}acetic acid hydrochloride (62.0 mg, crude) was isolated as beige solid.

LCMS (ESI+): m/z 358.8 [M+H]⁺

### Step E

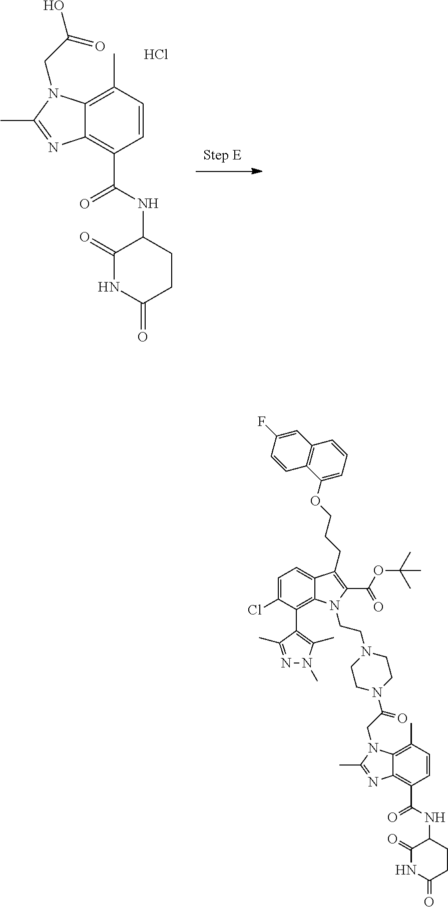 [SMILES: Cc1ccc(C(=O)NC2CCC(=O)NC2=O)c2nc(C)n(CC(=O)O)c12 ; Cl ; Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)ccc(C)c54)CC3)c12]

To a solution of tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (30.0 mg, 0.044 mmol), 2-{4-[(2,6-dioxopiperidin-3-yl)carbamoyl]-2,7-dimethyl-1H-1,3-benzodiazol-1-yl}acetic acid hydrochloride (21.1 mg, 0.053 mmol) and HATU (33.8 mg, 0.089 mmol) in dry DMF (1.7 mL) was added DIPEA (0.023 mL, 0.133 mmol). The mixture was stirred at room temperature for 30 min. The crude was diluted with DCM and washed with brine. The organic layer was dried over Na₂SO₄, filtered and concentrated in vacuo to get 51 mg crude with tert-butyl 6-chloro-1-{2-[4-(2-{4-[(2,6-dioxopiperidin-3-yl)carbamoyl]-2,7-dimethyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate as orange oil which was used in the next step without further purification.

LCMS (ESI+): m/z 1013.6 [M+H]⁺

### Step F

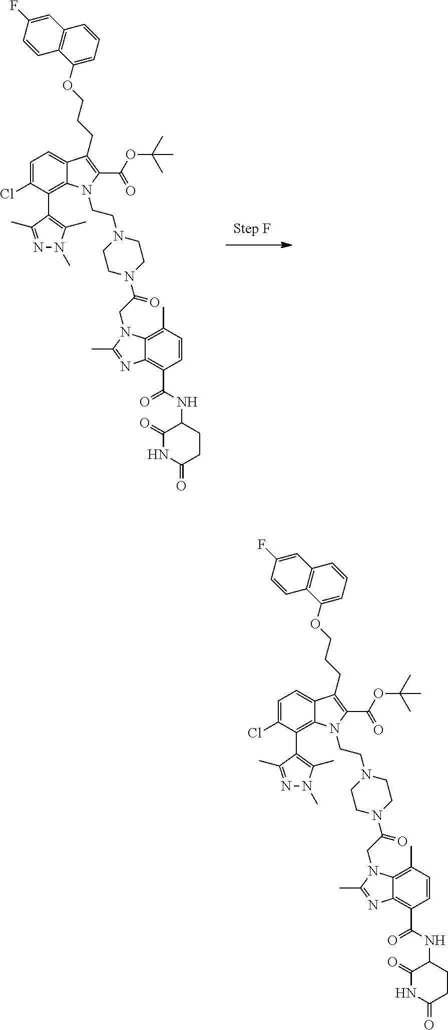 [SMILES: Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)ccc(C)c54)CC3)c12 ; Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)ccc(C)c54)CC3)c12]

To a solution of tert-butyl 6-chloro-1-{2-[4-(2-{4-[(2,6-dioxopiperidin-3-yl)carbamoyl]-2,7-dimethyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (51 mg, crude) in DCM (1.0 mL) was added TFA (1.0 mL, 13.059 mmol). The mixture was stirred for 18 h at RT. The crude was concentrated in vacuo and purified by preparative HPLC (C18, H₂O:MeCN+0.1% FA) to obtain 6-chloro-1-{2-[4-(2-{4-[(2,6-dioxopiperidin-3-yl)carbamoyl]-2,7-dimethyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (8.5 mg, 0.009 mmol, 20% over two steps) as a white solid.

LCMS (ESI+): m/z 958.2 [M+H]⁺

¹H NMR (500 MHz, DMSO, 353K) δ 10.54 (s, 1H), 10.27 (d, J=7.2 Hz, 1H), 8.25 (dd, J=9.3, 5.9 Hz, 1H), 7.75-7.72 (m, 2H), 7.59 (dd, J=10.4, 2.6 Hz, 1H), 7.45-7.40 (m, 2H), 7.35-7.30 (m, 1H), 7.24 (d, J=8.6 Hz, 1H), 7.00 (dd, J=7.7, 0.9 Hz, 1H), 6.88 (dd, J=5.6, 3.1 Hz, 1H), 5.30 (s, 2H), 4.83 (ddd, J=12.3, 7.2, 5.2 Hz, 1H), 4.33-4.19 (m, 4H), 3.78 (s, 3H), 3.50-3.41 (m, 4H), 3.32-3.28 (m, 2H), 2.80 (ddd, J=17.4, 12.9, 5.5 Hz, 1H), 2.64-2.57 (m, 1H), 2.54 (s, 3H), 2.34-2.06 (m, 10H), 2.03 (s, 3H), 1.91 (s, 3H).

3 protons in aliphatic area overlap with DMSO

### Example 117. 6-Chloro-1-(2-(4-(2-(7-chloro-4-((2,6-dioxopiperidin-3-yl)carbamoyl)-2-methyl-1H-benzo[d]imidazol-1-yl)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (271)

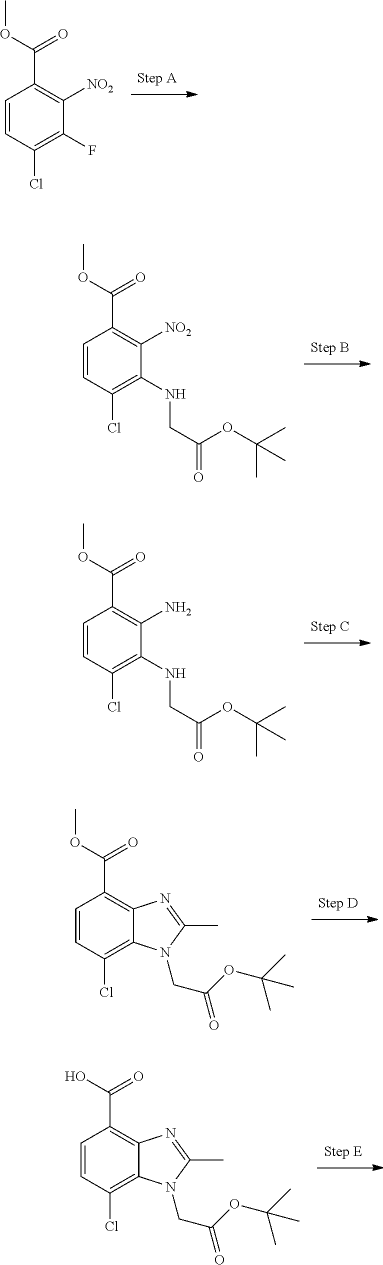 [SMILES: COC(=O)c1ccc(Cl)c(F)c1[N+](=O)[O-] ; COC(=O)c1ccc(Cl)c(NCC(=O)OC(C)(C)C)c1[N+](=O)[O-] ; COC(=O)c1ccc(Cl)c(NCC(=O)OC(C)(C)C)c1N ; COC(=O)c1ccc(Cl)c2c1nc(C)n2CC(=O)OC(C)(C)C ; Cc1nc2c(C(=O)O)ccc(Cl)c2n1CC(=O)OC(C)(C)C]

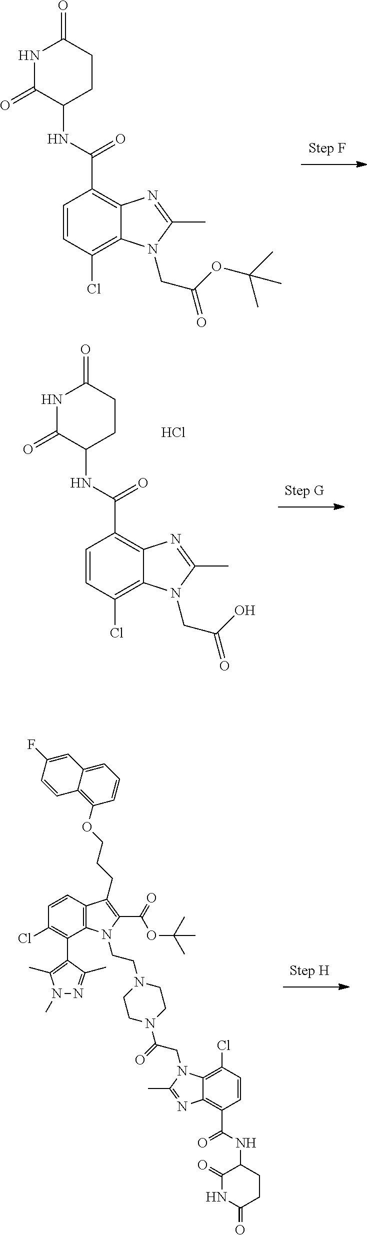

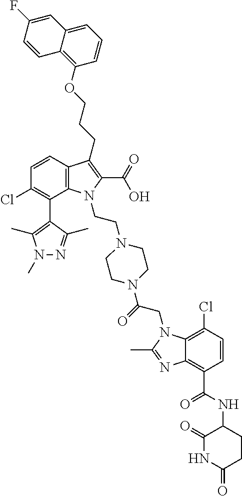 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)ccc(Cl)c54)CC3)c12]

### Step A

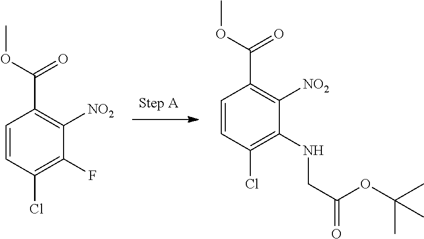 [SMILES: COC(=O)c1ccc(Cl)c(F)c1[N+](=O)[O-] ; COC(=O)c1ccc(Cl)c(NCC(=O)OC(C)(C)C)c1[N+](=O)[O-]]

To a stirring solution of methyl 4-chloro-3-fluoro-2-nitrobenzoate (1.7 g, 7.2 mmol) in DMSO (36 mL) were added tert-butyl glycinate (1.99 mL, 14.6 mmol) and DIPEA (3.643 mL, 21.888 mmol). The reaction mixture was then stirred for 2 h at 70° C. After complete consumption of the starting material, the reaction mixture was cooled to room temperature, diluted with EtOAc and washed with water followed and brine. The organic layer was dried over anhydrous Na₂SO₄, concentrated and purified by flash column chromatography (SiO₂, 20% EtOAc in hexane). Methyl 3-((2-(tert-butoxy)-2-oxoethyl)amino)-4-chloro-2-nitrobenzoate (1.4 g, 4.06 mmol, 56%) was obtained as yellow solid.

LCMS (ESI+): m/z 345.2 [M+H]⁺

### Step B

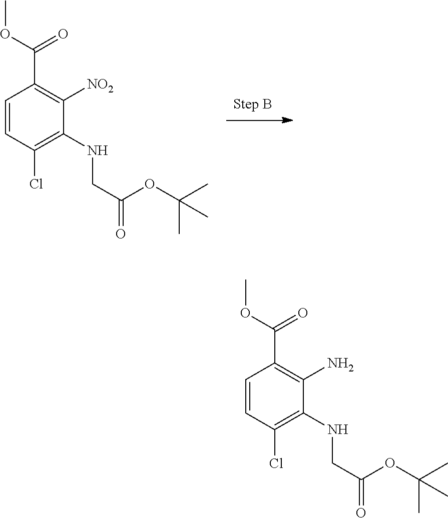 [SMILES: COC(=O)c1ccc(Cl)c(NCC(=O)OC(C)(C)C)c1[N+](=O)[O-] ; COC(=O)c1ccc(Cl)c(NCC(=O)OC(C)(C)C)c1N]

To a solution of methyl 3-((2-(tert-butoxy)-2-oxoethyl)amino)-4-chloro-2-nitrobenzoate (1.4 g, 4.06 mmol) in EtOAc (60 mL) was added platinum sulfided on carbon (700 mg). The reaction mixture was then stirred for 2 h at room temperature under hydrogen atmosphere. The reaction mixture was filtered through Celite® pad and washed with EtOAc. The filtrate was concentrated under reduced pressure to get methyl 2-amino-3-((2-(tert-butoxy)-2-oxoethyl)amino)-4-chlorobenzoate (1.0 g, crude) was used for the next step synthesis without further purification.

### Step C

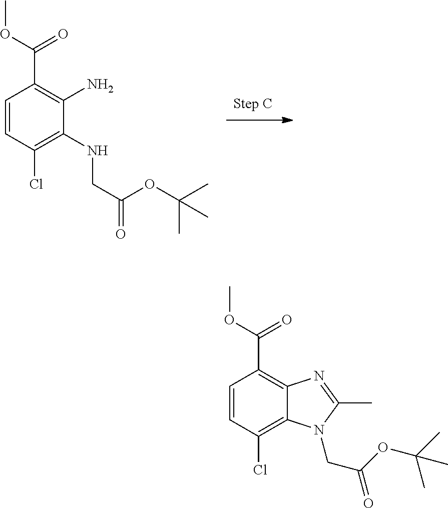 [SMILES: COC(=O)c1ccc(Cl)c(NCC(=O)OC(C)(C)C)c1N ; COC(=O)c1ccc(Cl)c2c1nc(C)n2CC(=O)OC(C)(C)C]

To a solution of crude methyl 2-amino-3-((2-(tert-butoxy)-2-oxoethyl)amino)-4-chlorobenzoate (1.0 g, crude) in HFIP (20 mL) was added 1,1,1-triethoxyethane (4.123 g, 25.415 mmol) the reaction mixture was then stirred for 16 h at RT. After complete consumption of the starting material, the volatiles were evaporated under reduced pressure to get the crude product which was then purified by column chromatography (SiO₂, 80% EtOAc in hexane) to afford methyl 1-(2-(tert-butoxy)-2-oxoethyl)-7-chloro-2-methyl-1H-benzo[d]imidazole-4-carboxylate (820 mg, 2.42 mmol, 59%) as off white solid.

LCMS (ESI+): m/z 339.15 [M+H]⁺

### Step D

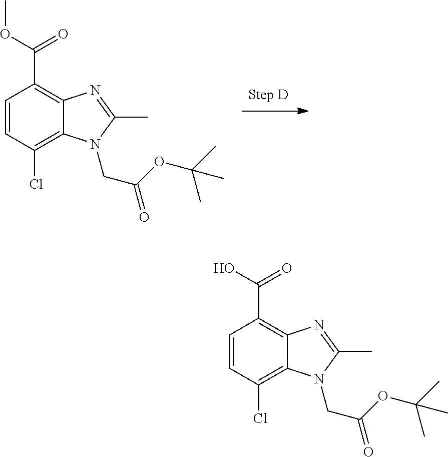 [SMILES: COC(=O)c1ccc(Cl)c2c1nc(C)n2CC(=O)OC(C)(C)C ; Cc1nc2c(C(=O)O)ccc(Cl)c2n1CC(=O)OC(C)(C)C]

Methyl 1-[2-(tert-butoxy)-2-oxoethyl]-7-chloro-2-methyl-1H-1,3-benzodiazole-4-carboxylate (40.0 mg, 0.118 mmol) was dissolved in MeCN (2.4 mL) and an aqueous solution of LiBr (0.59 mL, 205.1 mg, 2.361 mmol) was added, followed by Et₃N (0.197 mL, 1.417 mmol). Reaction, was stirred at room temperature for 72 h. After complete consumption of the starting material, the mixture was concentrated under reduced pressure, diluted with DMSO and purified with reverse phase flash chromatography (C18, H₂O:MeCN+0.1% FA) to give 1-[2-(tert-butoxy)-2-oxoethyl]-7-chloro-2-methyl-1H-1,3-benzodiazole-4-carboxylic acid (38.1 mg, 0.117 mmol, 99.4%) as white solid.

LCMS (ESI+): m/z 324.7 [M+H]⁺

### Step E

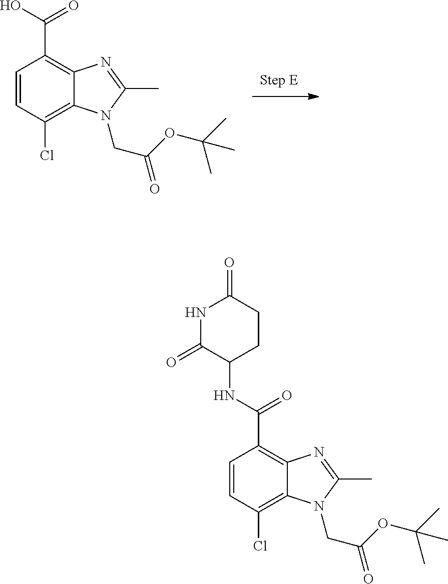 [SMILES: Cc1nc2c(C(=O)O)ccc(Cl)c2n1CC(=O)OC(C)(C)C ; Cc1nc2c(C(=O)NC3CCC(=O)NC3=O)ccc(Cl)c2n1CC(=O)OC(C)(C)C]

1-[2-(tert-Butoxy)-2-oxoethyl]-7-chloro-2-methyl-1H-1,3-benzodiazole-4-carboxylic acid (38.1 mg, 0.117 mmol) was dissolved in dry DMF (2.3 mL) under argon atmosphere and DIPEA (0.061 mL, 0.352 mmol) was added, followed by HATU (53.5 mg, 0.141 mmol) and 3-aminopiperidine-2,6-dione hydrochloride (29.0 mg, 0.176 mmol). Reaction was stirred for 1 h at room temperature. After complete consumption of the starting material, the solvent was evaporated under reduced pressure. The resulting residue was dissolved in DMSO and purified with reverse phase flash chromatography (C18, H₂O:MeCN+0.1% FA) to give tert-butyl 2-{7-chloro-4-[(2,6-dioxopiperidin-3-yl)carbamoyl]-2-methyl-1H-1,3-benzodiazol-1-yl}acetate (37.1 mg, 0.085 mmol, 72.7%) as white solid.

LCMS (ESI+): m/z 435.1 [M+H]⁺

### Step F

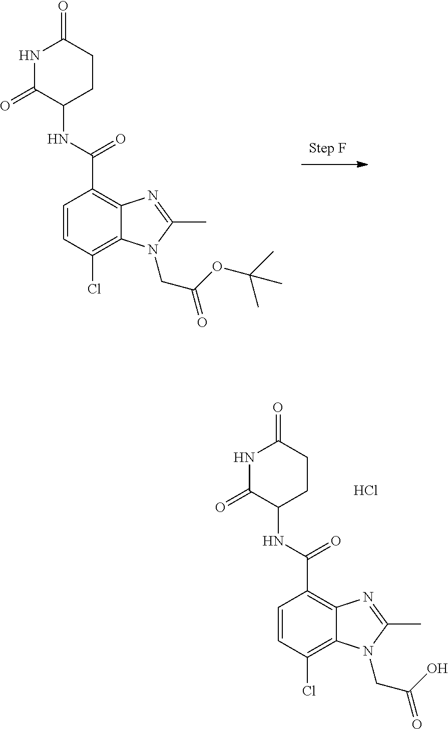 [SMILES: Cc1nc2c(C(=O)NC3CCC(=O)NC3=O)ccc(Cl)c2n1CC(=O)OC(C)(C)C ; Cc1nc2c(C(=O)NC3CCC(=O)NC3=O)ccc(Cl)c2n1CC(=O)O ; Cl]

tert-Butyl 2-{7-chloro-4-[(2,6-dioxopiperidin-3-yl)carbamoyl]-2-methyl-1H-1,3-benzodiazol-1-yl}acetate (33.3 mg, 0.077 mmol) was dissolved in dry DCM (1.0 mL) under argon atmosphere and TFA (1.0 mL, 13.059 mmol) was added. Reaction, was stirred for 16 h at room temperature. After complete consumption of the starting material, solvent along with TFA were evaporated under reduced pressure and an aqueous solution of 1M HCl (2 mL) was added. The solution was concentrated to dryness and the procedure was repeated twice. The resulting residue was dissolved in water and freeze-dried to give 2-{7-chloro-4-[(2,6-dioxopiperidin-3-yl)carbamoyl]-2-methyl-1H-1,3-benzodiazol-1-yl}acetic acid hydrochloride (31.6 mg, 0.076 mmol, 99.4%).

LCMS (ESI+): m/z 379.5

### Step G

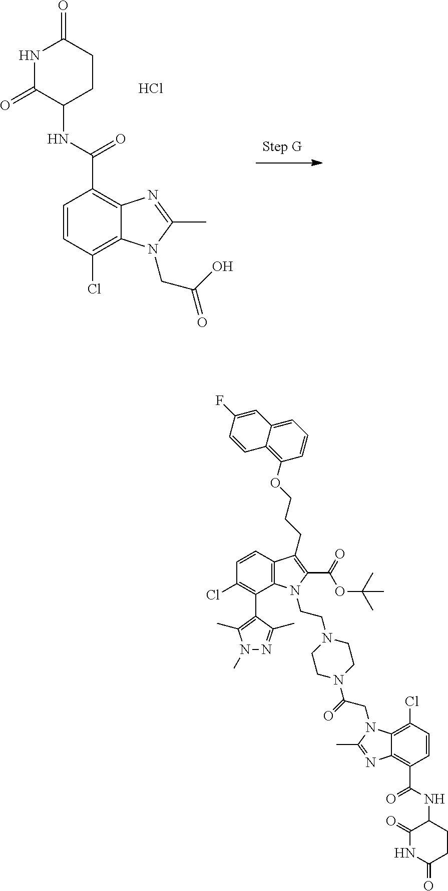 [SMILES: Cc1nc2c(C(=O)NC3CCC(=O)NC3=O)ccc(Cl)c2n1CC(=O)O ; Cl ; Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)ccc(Cl)c54)CC3)c12]

2-{7-Chloro-4-[(2,6-dioxopiperidin-3-yl)carbamoyl]-2-methyl-1H-1,3-benzodiazol-1-yl}acetic acid hydrochloride (27.1 mg, 0.065 mmol) was dissolved in dry DMF (0.6 mL) under argon atmosphere and DIPEA (0.031 mL, 0.178 mmol), followed by tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (40.0 mg, 0.059 mmol). To it, HATU (23.7 mg, 0.062 mmol) was added dropwise as a solution in DMF (0.6 mL) and the reaction, was stirred for 15 min. After complete consumption of the starting material, reaction mixture was diluted with DCM and poured into brine. The aqueous layer was extracted with DCM and the combined organics were washed with aqueous saturated NaHCO₃, again brine and water. Organic layer was dried over anhydrous Na₂SO₄, filtrated and dried under reduced pressure. Crude tert-butyl 6-chloro-1-{2-[4-(2-{7-chloro-4-[(2,6-dioxopiperidin-3-yl)carbamoyl]-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (54.7 mg) was used in the next step without further purification.

LCMS (ESI+): m/z 1034.9 m/z [M+H]⁺

### Step H

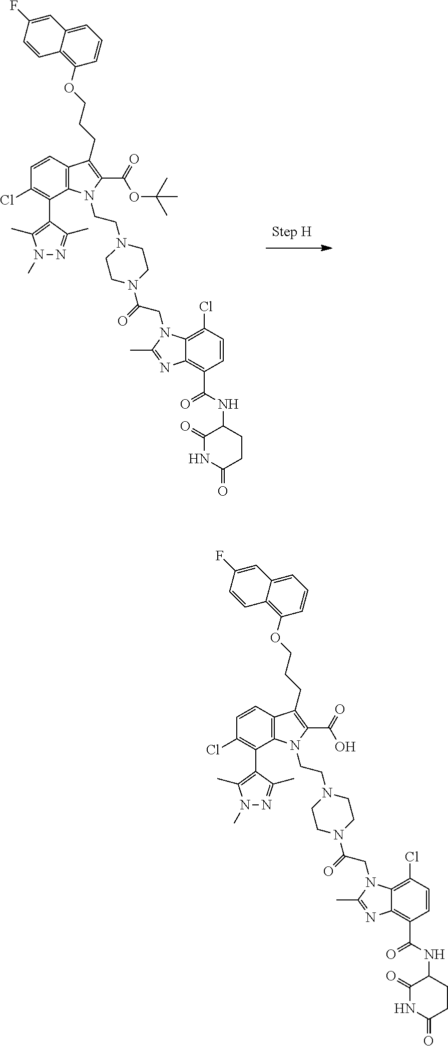 [SMILES: Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)ccc(Cl)c54)CC3)c12 ; Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)ccc(Cl)c54)CC3)c12]

tert-Butyl 6-chloro-1-{2-[4-(2-{7-chloro-4-[(2,6-dioxopiperidin-3-yl)carbamoyl]-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (54.7 mg, crude) was dissolved in dry DCM (0.500 mL) under argon atmosphere and TFA (0.500 mL, 6.529 mmol) was added. The reaction was stirred under argon atmosphere at room temperature for 16 h. After complete consumption of the starting material, mixture was concentrated with strong argon stream and dried under reduced pressure. The resulting residue was dissolved in DMSO and purified with preparative HPLC (C18, H₂O:MeCN+0.1% FA) to give 6-chloro-1-{2-[4-(2-{7-chloro-4-[(2,6-dioxopiperidin-3-yl)carbamoyl]-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (27.4 mg, 0.028 mmol, 47% over 2 steps) as white powder.

LCMS (ESI+): m/z 978.4 [M+H]⁺

¹H NMR (500 MHz, DMSO, 353 K) δ 10.60 (s, 1H), 10.14 (d, J=7.1 Hz, 1H), 8.26 (dd, J=9.3, 5.9 Hz, 1H), 7.85 (d, J=8.3 Hz, 1H), 7.75 (d, J=8.5 Hz, 1H), 7.60 (dd, J=10.4, 2.6 Hz, 1H), 7.49-7.39 (m, 2H), 7.34 (td, J=8.9, 2.6 Hz, 1H), 7.30 (d, J=8.3 Hz, 1H), 7.25 (d, J=8.5 Hz, 1H), 6.90 (dd, J=5.3, 3.3 Hz, 1H), 5.44 (s, 2H), 4.87 (ddd, J=12.3, 7.1, 5.2 Hz, 1H), 4.42-4.16 (m, 4H), 3.80 (s, 3H), 3.58-3.36 (m, 4H), 3.32 (dd, J=8.4, 6.5 Hz, 2H), 2.82 (ddd, J=17.4, 13.1, 5.5 Hz, 1H), 2.65-2.59 (m, 1H), 2.55 (s, 3H), 2.37-2.31 (m, 1H), 2.31-2.24 (m, 2H), 2.24-2.07 (m, 7H), 2.05 (s, 3H), 1.92 (s, 3H)

### Example 118. 6-Chloro-1-(2-(4-(2-(4-((2,6-dioxopiperidin-3-yl)carbamoyl)-7-methoxy-2-methyl-1H-benzo[d]imidazol-1-yl)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (272)

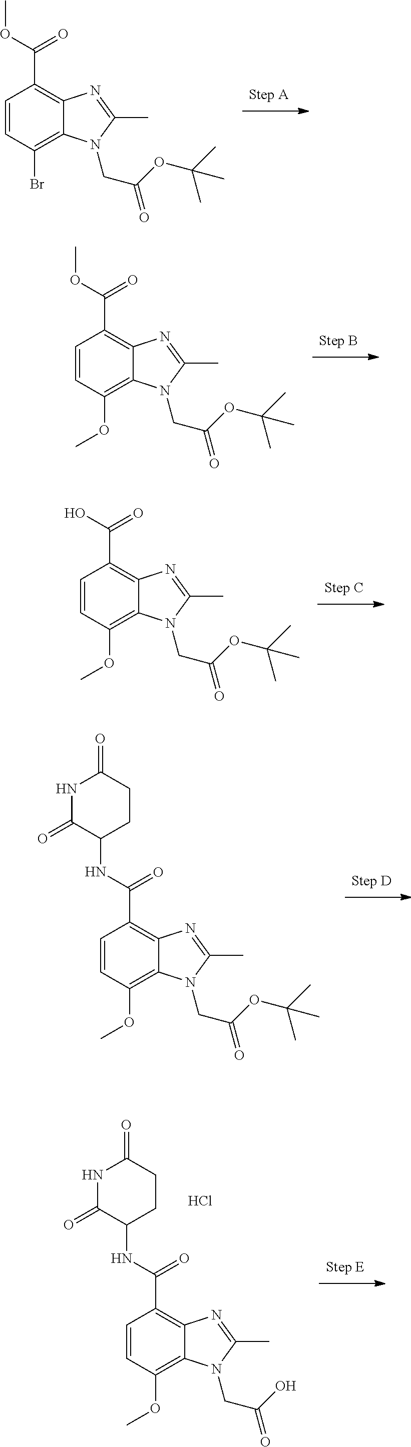 [SMILES: COC(=O)c1ccc(Br)c2c1nc(C)n2CC(=O)OC(C)(C)C ; COC(=O)c1ccc(OC)c2c1nc(C)n2CC(=O)OC(C)(C)C ; COc1ccc(C(=O)O)c2nc(C)n(CC(=O)OC(C)(C)C)c12 ; COc1ccc(C(=O)NC2CCC(=O)NC2=O)c2nc(C)n(CC(=O)OC(C)(C)C)c12 ; COc1ccc(C(=O)NC2CCC(=O)NC2=O)c2nc(C)n(CC(=O)O)c12 ; Cl]

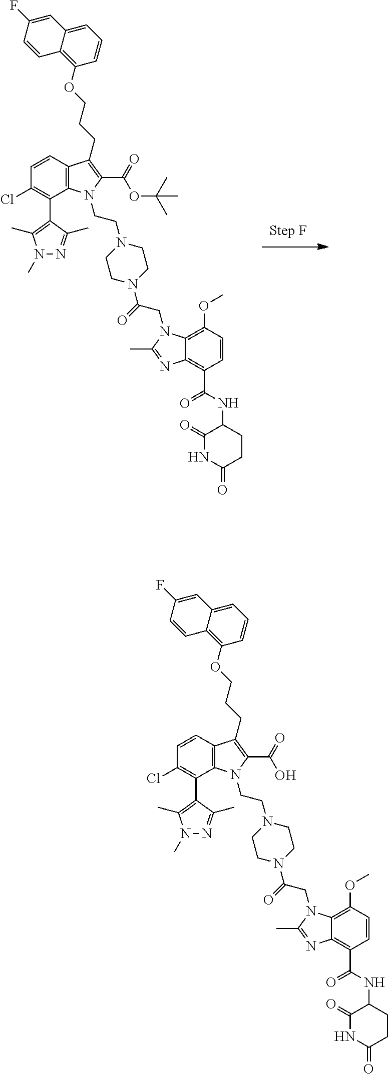 [SMILES: COc1ccc(C(=O)NC2CCC(=O)NC2=O)c2nc(C)n(CC(=O)N3CCN(CCn4c(C(=O)OC(C)(C)C)c(CCCOc5cccc6cc(F)ccc56)c5ccc(Cl)c(-c6c(C)nn(C)c6C)c54)CC3)c12 ; COc1ccc(C(=O)NC2CCC(=O)NC2=O)c2nc(C)n(CC(=O)N3CCN(CCn4c(C(=O)O)c(CCCOc5cccc6cc(F)ccc56)c5ccc(Cl)c(-c6c(C)nn(C)c6C)c54)CC3)c12]

### Step A

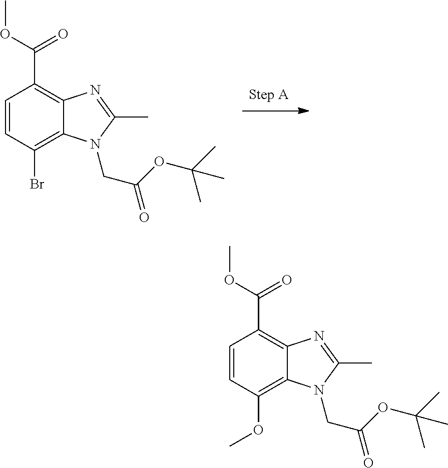 [SMILES: COC(=O)c1ccc(Br)c2c1nc(C)n2CC(=O)OC(C)(C)C ; COC(=O)c1ccc(OC)c2c1nc(C)n2CC(=O)OC(C)(C)C]

To a stirred solution of methyl 7-bromo-1-(2-(tert-butoxy)-2-oxoethyl)-2-methyl-1H-benzo[d]imidazole-4-carboxylate (2.0 g, 5.2 mmol) were added MeOH (0.635 mL) followed by Cs₂CO₃ (3.40 g, 10.47 mmol). The reaction mixture was then sparged with argon for 10 min. Adamantyl-BippyPhos (520 mg, 0.785 mmol) and Pd₂(dba)₃ (479 mg, 0.52 mmol) were then added and the reaction mixture again was sparged with argon for 5 min. Finally the reaction mixture was stirred at 100° C. for 20 h in a sealed tube. After complete consumption of the starting material, the reaction mixture was cooled to room temperature and then filtered through Celite® pad. The filtrate was concentrated under reduced pressure to get the crude material, which was purified by column chromatography (SiO₂, 3% MeOH in DCM) to afford methyl 1-[2-(tert-butoxy)-2-oxoethyl]-7-methoxy-2-methyl-1H-1,3-benzodiazole-4-carboxylate (564 mg, 1.7 mmol, 33%) as off white solid.

LCMS (ESI+): m/z 335.2 [M+H]⁺

### Step B

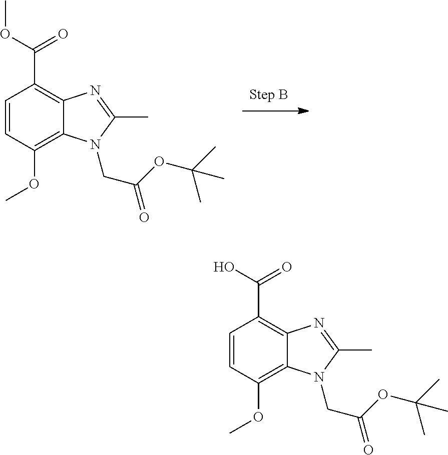 [SMILES: COC(=O)c1ccc(OC)c2c1nc(C)n2CC(=O)OC(C)(C)C ; COc1ccc(C(=O)O)c2nc(C)n(CC(=O)OC(C)(C)C)c12]

Methyl 1-[2-(tert-butoxy)-2-oxoethyl]-7-methoxy-2-methyl-1H-1,3-benzodiazole-4-carboxylate (75.0 mg, 0.224 mmol) was dissolved in MeCN (4.5 mL) and 1 mL of aqueous solution of LiBr (389.6 mg, 4.486 mmol) was added, followed by Et₃N (0.374 mL, 2.692 mmol). Reaction was stirred at room temperature for 6 days. Then, the mixture was evaporated under reduced pressure to dryness and the residue was dissolved in THE (4.5 mL). Again, aqueous LiBr (1.0 mL, 389.6 mg, 4.486 mmol) and Et₃N (0.374 mL, 2.692 mmol) were added and the reaction was continued for the next 48 h at room temperature. After no further reaction progress was noted, the mixture was concentrated under reduced pressure, diluted with DMSO and purified with reverse phase flash chromatography (C18, H₂O:MeCN+0.1% FA) to give 1-[2-(tert-butoxy)-2-oxoethyl]-7-methoxy-2-methyl-1H-1,3-benzodiazole-4-carboxylic acid (30.0 mg, 0.094 mmol, 41.8%) as white solid.

LCMS (ESI+): m/z 321.0 [M+H]⁺

### Step C

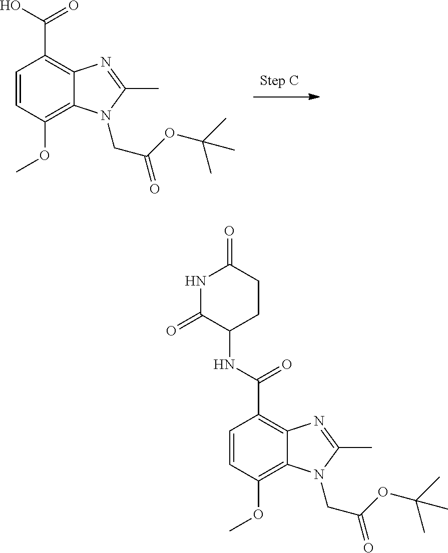 [SMILES: COc1ccc(C(=O)O)c2nc(C)n(CC(=O)OC(C)(C)C)c12 ; COc1ccc(C(=O)NC2CCC(=O)NC2=O)c2nc(C)n(CC(=O)OC(C)(C)C)c12]

1-[2-(tert-Butoxy)-2-oxoethyl]-7-methoxy-2-methyl-1H-1,3-benzodiazole-4-carboxylic acid (30.0 mg, 0.094 mmol) was dissolved in dry DMF (3.1 mL) under argon atmosphere and DIPEA (0.049 mL, 0.281 mmol) followed by HATU (42.7 mg, 0.112 mmol) were added. Reaction was stirred for 15 min at room temperature and 3-aminopiperidine-2,6-dione hydrochloride (23.1 mg, 0.140 mmol) was added. Reaction mixture was stirred at room temperature for 20 min. After complete consumption of the starting material, DMF was evaporated under reduced pressure. Resulting residue was dissolved in DMSO and reaction product was purified with reverse phase flash chromatography (C18, H₂O:MeCN+0.1% FA) to give tert-butyl 2-(4-((2,6-dioxopiperidin-3-yl)carbamoyl)-7-methoxy-2-methyl-1H-benzo[d]imidazol-1-yl)acetate (25.1 mg, 0.058 mmol, 62.3%) as white solid.

LCMS (ESI+): m/z 431.5 [M+H]⁺

### Step D

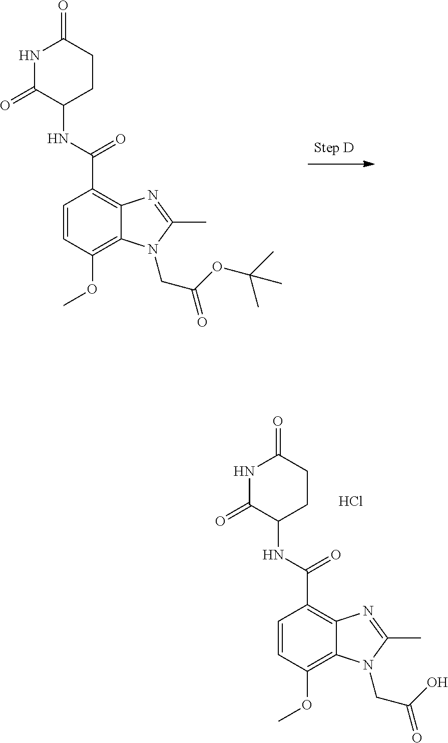 [SMILES: COc1ccc(C(=O)NC2CCC(=O)NC2=O)c2nc(C)n(CC(=O)OC(C)(C)C)c12 ; COc1ccc(C(=O)NC2CCC(=O)NC2=O)c2nc(C)n(CC(=O)O)c12 ; Cl]

tert-Butyl 2-(4-((2,6-dioxopiperidin-3-yl)carbamoyl)-7-methoxy-2-methyl-1H-benzo[d]imidazol-1-yl)acetate (23.1 mg, 0.054 mmol) was dissolved in dry DCM (0.5 mL) under argon atmosphere and TFA (0.500 mL, 6.529 mmol) was added. Reaction was stirred for 16 h at room temperature. After complete consumption of the starting material, solvent along with TFA were evaporated under reduced pressure and an aqueous solution of 1M HCl (2 mL) was added. The solution was concentrated to dryness and the procedure was repeated twice. The resulting residue was dissolved in water and freeze-dried to give 2-{4-[(2,6-dioxopiperidin-3-yl)carbamoyl]-7-methoxy-2-methyl-1H-1,3-benzodiazol-1-yl}acetic acid hydrochloride (22.0 mg, 0.054 mmol, 99.8%), which was used directly in the next step.

LCMS (ESI+): m/z 374.7 [M+H]⁺

### Step E

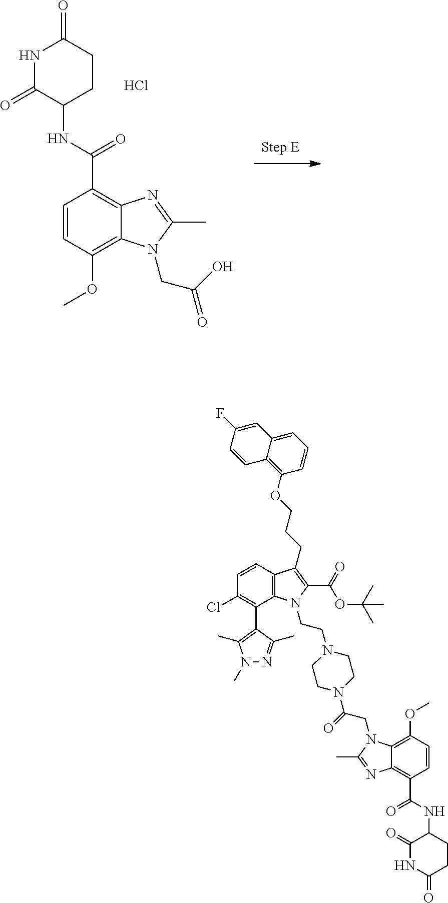 [SMILES: COc1ccc(C(=O)NC2CCC(=O)NC2=O)c2nc(C)n(CC(=O)O)c12 ; Cl ; COc1ccc(C(=O)NC2CCC(=O)NC2=O)c2nc(C)n(CC(=O)N3CCN(CCn4c(C(=O)OC(C)(C)C)c(CCCOc5cccc6cc(F)ccc56)c5ccc(Cl)c(-c6c(C)nn(C)c6C)c54)CC3)c12]

2-{4-[(2,6-Dioxopiperidin-3-yl)carbamoyl]-7-methoxy-2-methyl-1H-1,3-benzodiazol-1-yl}acetic acid hydrochloride (23.5 mg, 0.057 mmol) was dissolved in dry DMF (2.0 mL) under argon atmosphere and DIPEA (0.027 mL, 0.156 mmol), followed by tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (35.0 mg, 0.052 mmol). To it, HATU (20.7 mg, 0.055 mmol) was added dropwise as a solution in DMF (0.6 mL) and the reaction was stirred for 15 min. After complete consumption of the starting material, reaction mixture was diluted with DCM and poured into brine. The aqueous layer was extracted with DCM and the combined organics were washed with aqueous saturated NaHCO₃, again brine and water. Organic layer was dried over anhydrous Na₂SO₄, filtrated and dried under reduced pressure. Crude tert-butyl 6-chloro-1-{2-[4-(2-{4-[(2,6-dioxopiperidin-3-yl)carbamoyl]-7-methoxy-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate was used in the next step without further purification.

LCMS (ESI+): m/z 1029.7 [M+H]⁺

### Step F

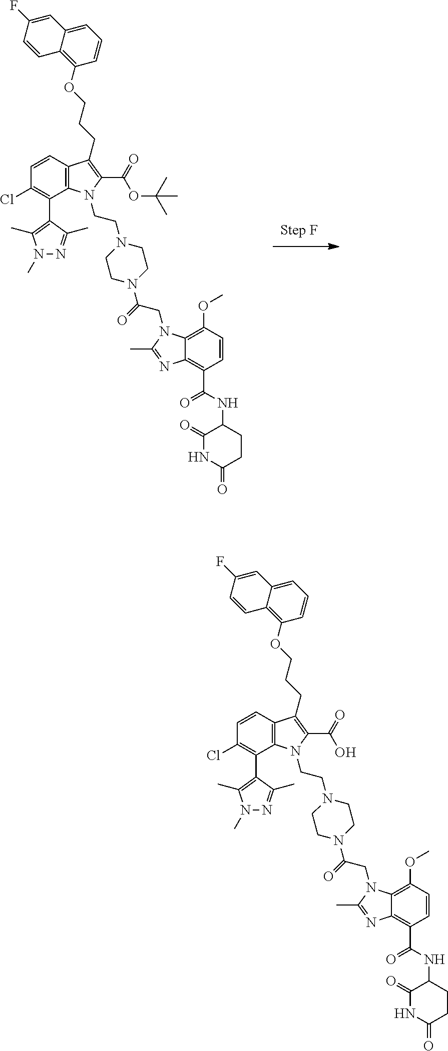 [SMILES: COc1ccc(C(=O)NC2CCC(=O)NC2=O)c2nc(C)n(CC(=O)N3CCN(CCn4c(C(=O)OC(C)(C)C)c(CCCOc5cccc6cc(F)ccc56)c5ccc(Cl)c(-c6c(C)nn(C)c6C)c54)CC3)c12 ; COc1ccc(C(=O)NC2CCC(=O)NC2=O)c2nc(C)n(CC(=O)N3CCN(CCn4c(C(=O)O)c(CCCOc5cccc6cc(F)ccc56)c5ccc(Cl)c(-c6c(C)nn(C)c6C)c54)CC3)c12]

Crude tert-butyl 6-chloro-1-{2-[4-(2-{4-[(2,6-dioxopiperidin-3-yl)carbamoyl]-7-methoxy-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (72.2 mg) was dissolved in dry DCM (0.537 mL) under argon atmosphere and TFA (0.537 mL, 7.010 mmol) was added. Reaction was stirred under argon atmosphere at room temperature for 16 h. After complete consumption of the starting material, mixture was concentrated with strong argon stream and dried under reduced pressure. The resulting residue was dissolved in DMSO and purified with preparative HPLC (C18, H₂O:MeCN+0.1% FA) to give 6-chloro-1-(2-(4-(2-(4-((2,6-dioxopiperidin-3-yl)carbamoyl)-7-methoxy-2-methyl-1H-benzo[d]imidazol-1-yl)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (12.0 mg, 0.012 mmol, 23% over 2 steps) as white powder.

LCMS (ESI+): m/z 974.6 [M+H]⁺

¹H NMR (500 MHz, DMSO, 353 K) δ 10.56 (s, 1H), 10.05 (d, J=7.2 Hz, 1H), 8.26 (dd, J=9.3, 5.9 Hz, 1H), 7.82 (d, J=8.4 Hz, 1H), 7.79-7.71 (m, 1H), 7.60 (dd, J=10.4, 2.6 Hz, 1H), 7.50-7.39 (m, 2H), 7.34 (td, J=8.9, 2.6 Hz, 1H), 7.29-7.22 (m, 1H), 6.90 (dd, J=5.4, 3.2 Hz, 1H), 6.86 (d, J=8.6 Hz, 1H), 5.35-5.24 (m, 2H), 4.84 (ddd, J=12.3, 7.2, 5.2 Hz, 1H), 4.38-4.19 (m, 4H), 3.92 (s, 3H), 3.80 (s, 3H), 3.55-3.36 (m, 4H), 3.35-3.26 (m, 2H), 2.81 (ddd, J=17.4, 13.0, 5.5 Hz, 1H), 2.65-2.59 (m, 1H), 2.48 (s, 3H), 2.36-2.24 (m, 3H), 2.24-2.06 (m, 7H), 2.05 (s, 3H), 1.93 (s, 3H).

### Example 119: 1-{2-[4-(2-{6-Bromo-4-[(2,6-dioxopiperidin-3-yl)carbamoyl]-2-methyl-1H-1,3-benzodiazol-1-yl}acetyl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylic acid (273)

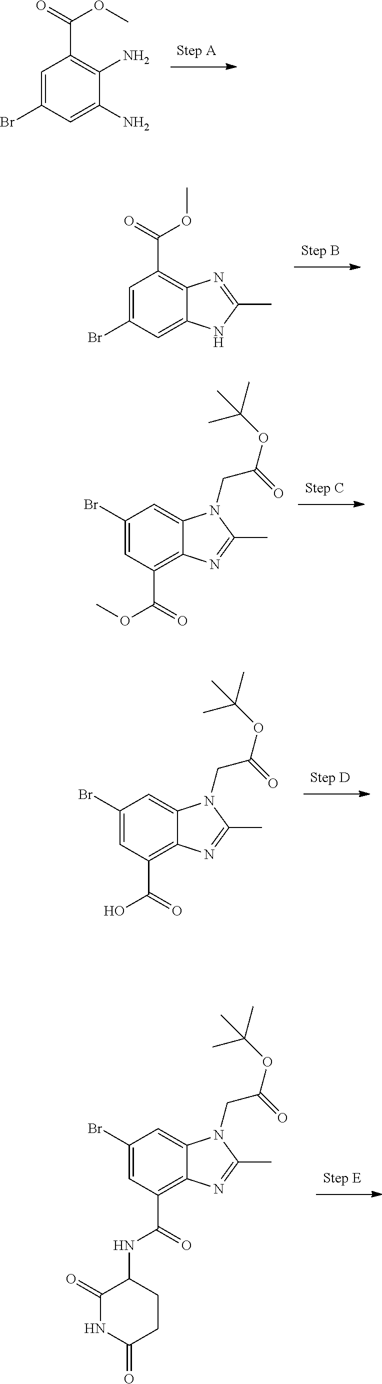 [SMILES: COC(=O)c1cc(Br)cc(N)c1N ; COC(=O)c1cc(Br)cc2[nH]c(C)nc12 ; COC(=O)c1cc(Br)cc2c1nc(C)n2CC(=O)OC(C)(C)C ; Cc1nc2c(C(=O)O)cc(Br)cc2n1CC(=O)OC(C)(C)C ; Cc1nc2c(C(=O)NC3CCC(=O)NC3=O)cc(Br)cc2n1CC(=O)OC(C)(C)C]

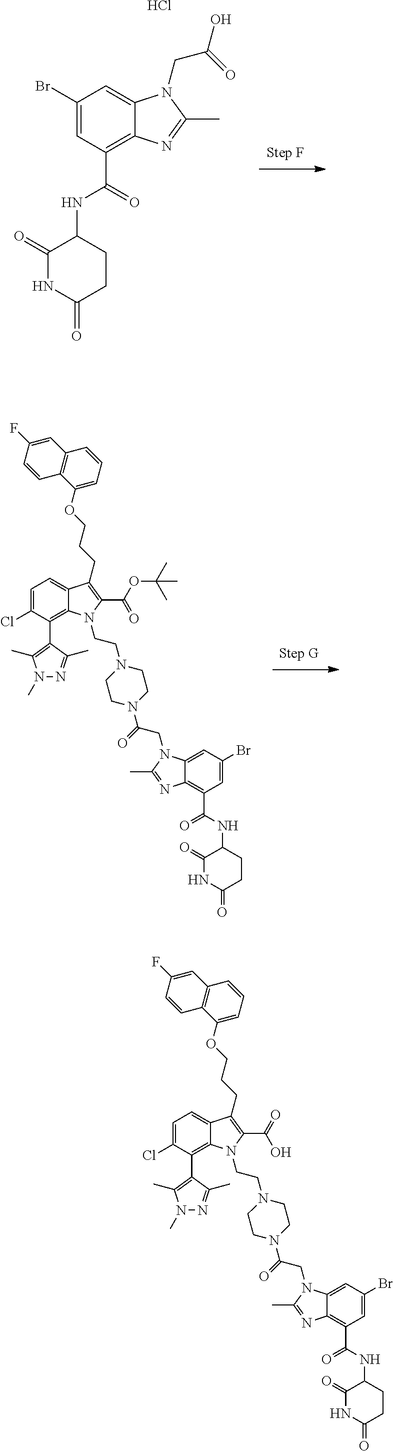 [SMILES: Cl ; Cc1nc2c(C(=O)NC3CCC(=O)NC3=O)cc(Br)cc2n1CC(=O)O ; Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)cc(Br)cc54)CC3)c12 ; Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cc(Br)cc54)CC3)c12]

### Step A

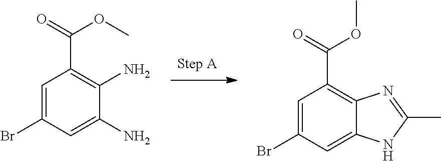

To a solution of methyl 2,3-diamino-5-bromobenzoate (4.0 g, 16.3 mmol) in AcOH (25 mL) was added Et₃N (1.33 mL, 8.16 mmol) at room temperature and the reaction mixture was heated to reflux for 16 h under nitrogen. After complete consumption of the starting material, the volatiles were evaporated under reduced pressure which was then dissolved in EtOAc and washed with water followed by brine solution. The organic layer was dried over anhydrous Na₂SO₄, concentrated and crude was purified by column chromatography (SiO₂, 80% EtOAc in hexane) to afford methyl 6-bromo-2-methyl-1H-benzo[d]imidazole-4-carboxylate (2.5 g, 9.29 mmol, 57%) as off white solid.

LCMS (ESI+): m/z 268.5 [M+H]⁺

### Step B

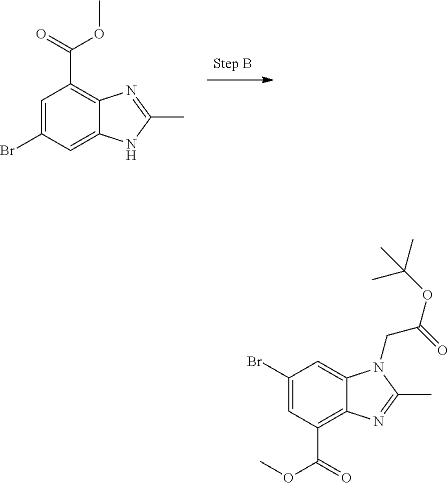 [SMILES: COC(=O)c1cc(Br)cc2[nH]c(C)nc12 ; COC(=O)c1cc(Br)cc2c1nc(C)n2CC(=O)OC(C)(C)C]

To a stirring solution of methyl 6-bromo-2-methyl-1H-benzo[d]imidazole-4-carboxylate (10.0 g, 37.2 mmol) in THE (250 mL) was added NaH (2.14 g, 55.7 mmol) portion wise at 0° C. under nitrogen. The reaction mixture was allowed to stir at the same temperature for 20 min. tert-Butyl bromoacetate (8.7 g, 44.6 mmol) was then added to the reaction mixture and stirred for 2 h at room temperature. After complete consumption of the starting material, the reaction mixture was quenched by using ice, it was then diluted with EtOAc and washed by water followed by brine. The organic layer was dried over anhydrous Na₂SO₄, concentrated and crude was purified by column chromatography (SiO₂, 80% EtOAc in hexane) to afford methyl 6-bromo-1-[2-(tert-butoxy)-2-oxoethyl]-2-methyl-1H-1,3-benzodiazole-4-carboxylate (9.0 g, 23.5 mmol, 63%) as off white solid.

LCMS (ESI+): m/z 383.2 & 385.2 [M+H]⁺

### Step C

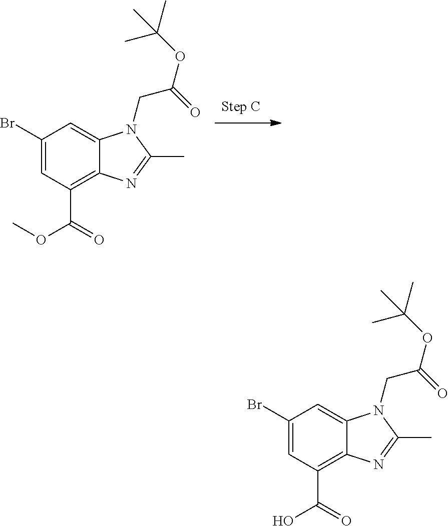 [SMILES: COC(=O)c1cc(Br)cc2c1nc(C)n2CC(=O)OC(C)(C)C ; Cc1nc2c(C(=O)O)cc(Br)cc2n1CC(=O)OC(C)(C)C]

Methyl 6-bromo-1-[2-(tert-butoxy)-2-oxoethyl]-2-methyl-1H-1,3-benzodiazole-4-carboxylate (105.0 mg, 0.274 mmol) was dissolved in MeCN (2.8 mL) and Et₃N (0.381 mL, 2.740 mmol) was added. To this mixture LiBr (475.8 mg, 5.480 mmol) dissolved in water (0.698 mL) was added and mixture was stirred at room temperature for 16 h. After this time, white precipitate was observed. Organic solvent was removed under reduced pressure, and precipitate was filtered, washed with water and dried under vacuum to give 6-bromo-1-[2-(tert-butoxy)-2-oxoethyl]-2-methyl-1H-1,3-benzodiazole-4-carboxylic acid (67.3 mg, 0.182 mmol, 66.5%) as off white solid.

LCMS (ESI+): m/z 368.95 & 370.6 [M+H]⁺

### Step D

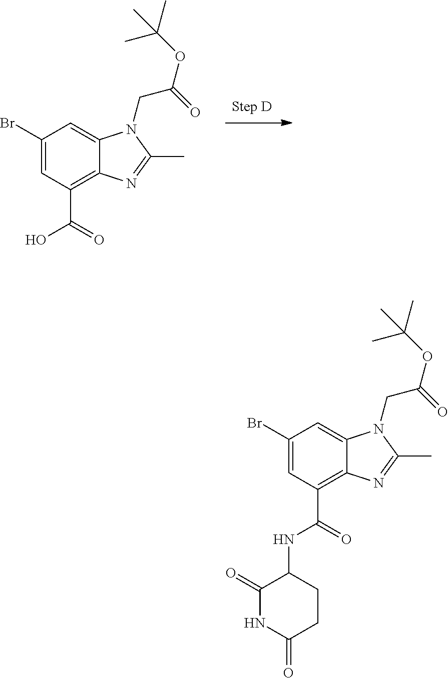 [SMILES: Cc1nc2c(C(=O)O)cc(Br)cc2n1CC(=O)OC(C)(C)C ; Cc1nc2c(C(=O)NC3CCC(=O)NC3=O)cc(Br)cc2n1CC(=O)OC(C)(C)C]

6-Bromo-1-[2-(tert-butoxy)-2-oxoethyl]-2-methyl-1H-1,3-benzodiazole-4-carboxylic acid (65.0 mg, 0.176 mmol) was dissolved in dry DMF (3.5 mL). DIPEA (0.092 mL, 0.528 mmol) followed by HATU (70.3 mg, 0.185 mmol) were added and the reaction mixture was stirred at room temperature for 15 min. 3-aminopiperidine-2,6-dione hydrochloride (58.0 mg, 0.352 mmol), was added and the reaction mixture was stirred for 30 min at RT. Reaction mixture was purified using reverse phase chromatography (C18, H₂O:MeCN+0.1% FA) to get tert-butyl 2-{6-bromo-4-[(2,6-dioxopiperidin-3-yl)carbamoyl]-2-methyl-1H-1,3-benzodiazol-1-yl}acetate (64.0 mg, 0.127 mmol, 72.3%) as white solid.

LCMS (ESI+): m/z 479.1 & 481.1 [M+H]⁺

### Step E

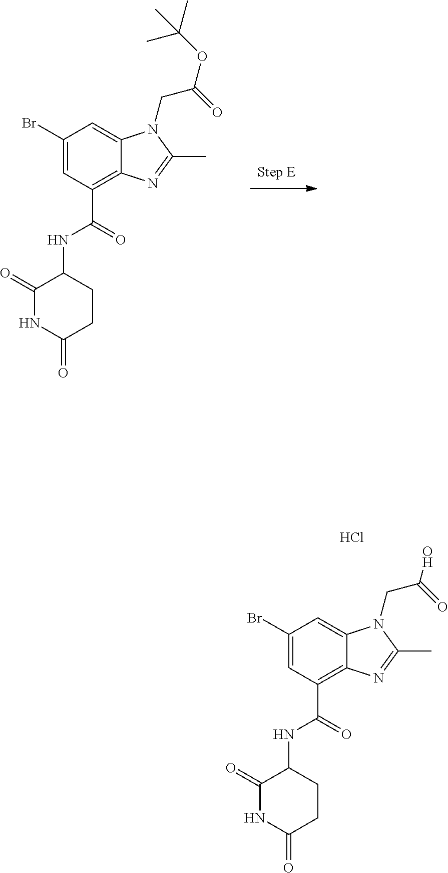 [SMILES: Cc1nc2c(C(=O)NC3CCC(=O)NC3=O)cc(Br)cc2n1CC(=O)OC(C)(C)C ; Cl ; Cc1nc2c(C(=O)NC3CCC(=O)NC3=O)cc(Br)cc2n1CC(=O)O]

To a solution of tert-butyl 2-{6-bromo-4-[(2,6-dioxopiperidin-3-yl)carbamoyl]-2-methyl-1H-1,3-benzodiazol-1-yl}acetate (58.0 mg, 0.121 mmol) in DCM (1.6 mL) was added TFA (0.162 mL, 2.109 mmol). The mixture was stirred for 72 h at RT. Solvents were removed under reduced pressure. The mixture was co-evaporated two times with 1M aqueous solution of HCl to give 2-{6-bromo-4-[(2,6-dioxopiperidin-3-yl)carbamoyl]-2-methyl-1H-1,3-benzodiazol-1-yl}acetic acid hydrochloride (49.5 mg, 0.108 mmol, 89.0%) as white solid.

### Step F

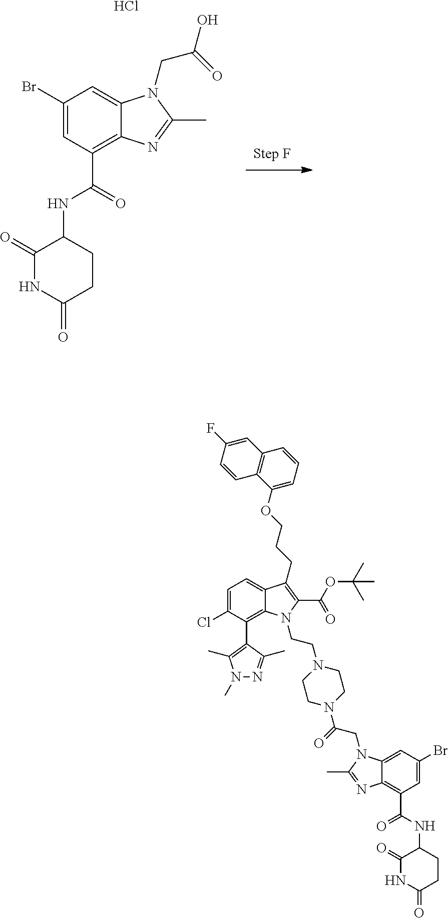 [SMILES: Cl ; Cc1nc2c(C(=O)NC3CCC(=O)NC3=O)cc(Br)cc2n1CC(=O)O ; Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)cc(Br)cc54)CC3)c12]

2-{6-Bromo-4-[(2,6-dioxopiperidin-3-yl)carbamoyl]-2-methyl-1H-1,3-benzodiazol-1-yl}acetic acid hydrochloride (22.5 mg, 0.049 mmol), tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (30.0 mg, 0.044 mmol) and HATU (33.8 mg, 0.089 mmol) were dissolved in dry DMF (1.1 mL) and to the mixture was added DIPEA (0.039 mL, 0.222 mmol). The reaction was stirred at room temperature for 2 h. The solvent was evaporated. Residues were dissolved in DCM and washed with brine. The organic layer was dried over Na₂SO₄, filtered and concentrated in vacuo. tert-Butyl 1-{2-[4-(2-{6-bromo-4-[(2,6-dioxopiperidin-3-yl)carbamoyl]-2-methyl-1H-1,3-benzodiazol-1-yl}acetyl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylate (35.0 mg, crude) was used without a further purification to next step.

LCMS (ESI+): m/z 1078.3 & 1080.25 [M+H]⁺

### Step G

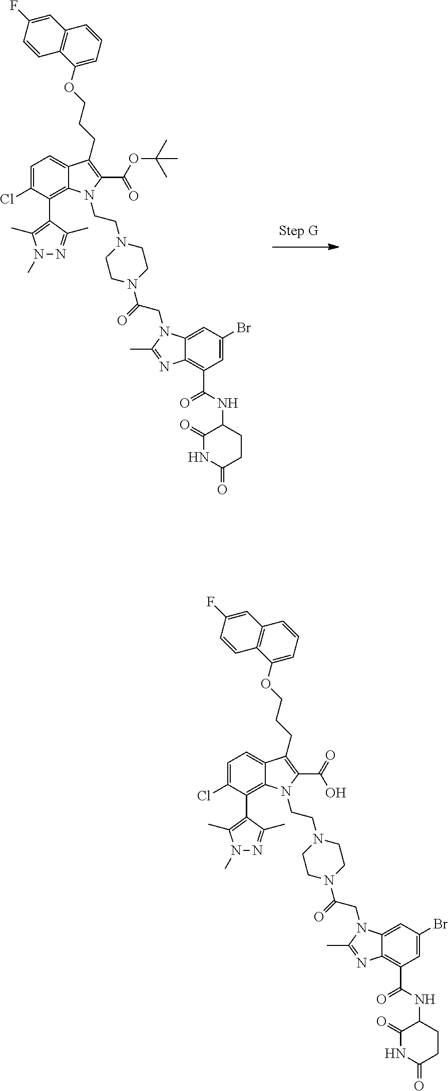 [SMILES: Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)cc(Br)cc54)CC3)c12 ; Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cc(Br)cc54)CC3)c12]

To solution of tert-butyl 1-{2-[4-(2-{6-bromo-4-[(2,6-dioxopiperidin-3-yl)carbamoyl]-2-methyl-1H-1,3-benzodiazol-1-yl}acetyl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylate (35.0 mg, crude) in DCM (107 μL) was added TFA (107 μL, 1.399 mmol), and mixture was stirred for 2 days at room temperature. Upon completion, the mixture was diluted with DCM and water was added, and organic solvent was removed under vacuum. Crude was purified by preparative HPLC (C18, H₂O:MeCN+0.1% FA) to give a corresponding 1-{2-[4-(2-{6-bromo-4-[(2,6-dioxopiperidin-3-yl)carbamoyl]-2-methyl-1H-1,3-benzodiazol-1-yl}acetyl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylic acid (8.8 mg, 0.009 mmol, 20% over 2 steps) as a white solid.

LCMS (ESI+): m/z 1022.2 [M+H]⁺

¹H NMR (500 MHz, DMSO, 353 K) δ=10.58 (s, 1H), 10.04 (d, J=7.2, 1H), 8.25 (dd, J=9.2, 5.9, 1H), 7.91 (q, J=1.9, 2H), 7.73 (d, J=8.5, 1H), 7.59 (dd, J=10.4, 2.6, 1H), 7.46-7.40 (m, 2H), 7.33 (td, J=8.9, 2.7, 1H), 7.23 (d, J=8.6, 1H), 6.88 (dd, J=5.4, 3.3, 1H), 5.24 (s, 2H), 4.86 (ddd, J=12.4, 7.2, 5.2, 1H), 4.35-4.17 (m, 4H), 3.78 (s, 3H), 3.43 (bs, 4H), 3.34-3.25 (m, 2H), 2.81 (ddd, J=17.5, 13.1, 5.5, 1H), 2.63-2.56 (m, 1H), 2.53-2.51 (m, 1H), 2.48 (s, 3H), 2.35-2.10 (m, 9H), 2.03 (s, 3H), 1.91 (s, 3H).

### Example 120. 6-Chloro-1-(2-(4-(2-(4-((2,6-dioxopiperidin-3-yl)carbamoyl)-2,6-dimethyl-1H-benzo[d]imidazol-1-yl)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (274)

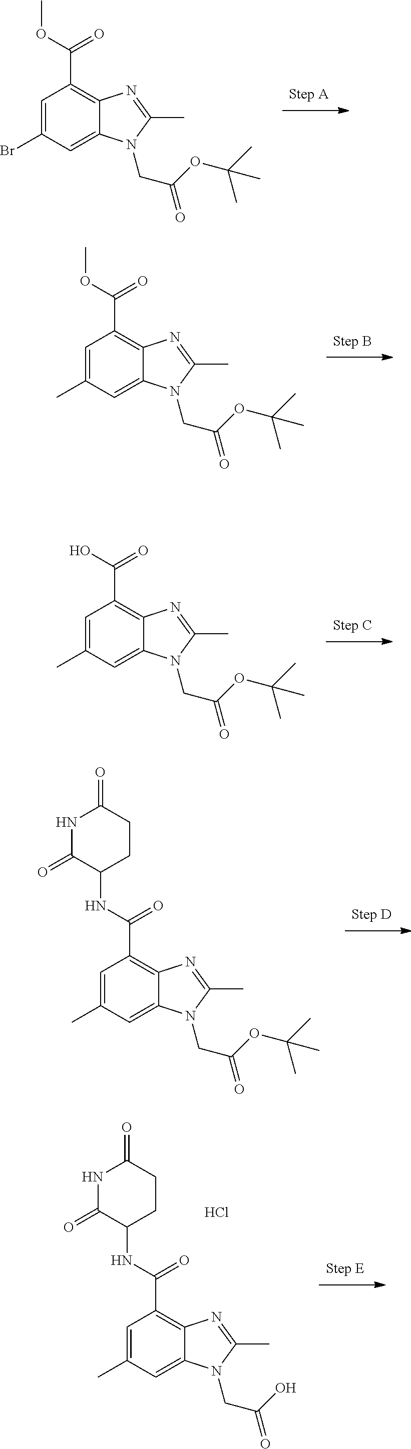

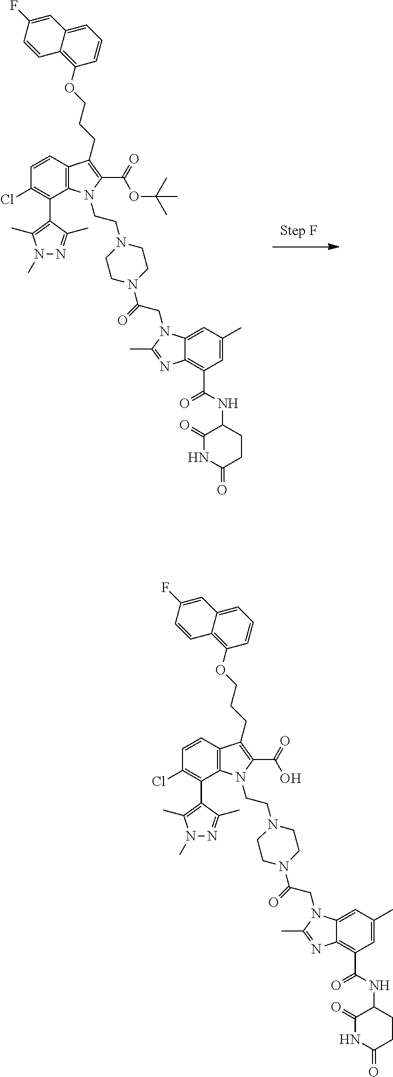

### Step A

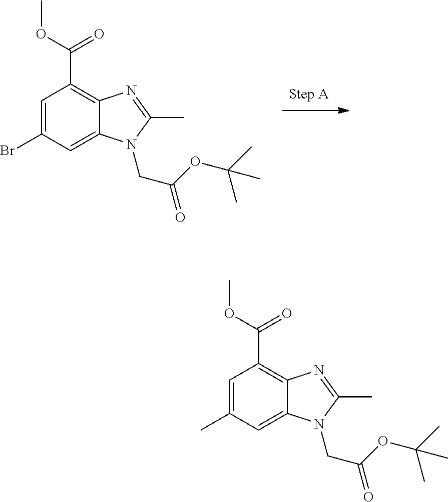 [SMILES: COC(=O)c1cc(Br)cc2c1nc(C)n2CC(=O)OC(C)(C)C ; COC(=O)c1cc(C)cc2c1nc(C)n2CC(=O)OC(C)(C)C]

To a stirred solution of methyl 6-bromo-1-(2-(tert-butoxy)-2-oxoethyl)-2-methyl-1H-benzo[d]imidazole-4-carboxylate (3.5 g, 9.13 mmol) in 1,4-dioxane (60 mL) were added methyl boronic acid (1.89 g, 31.98 mmol) and potassium phosphate (5.81 g, 27.42 mmol). The reaction mixture was then sparged with argon for 10 min. S—PHOS (1.12 g, 2.73 mmole) and Pd(OAc)₂ (307 mg, 1.37 mmol) were then added to the reaction mixture and the reaction vessel was again sparged with argon for 10 min. The reaction mixture was then stirred for 16 h at 90° C. After complete consumption of the starting material, the reaction mixture was then to room temperature and filtered through Celite®. The filtrate was concentrated under reduced pressure. Crude material was purified by column chromatography (SiO₂, 80% EtOAc in hexane) to afford 1.2 g of methyl 1-(2-(tert-butoxy)-2-oxoethyl)-2,6-dimethyl-1H-benzo[d]imidazole-4-carboxylate (3.77 mmol, 41%) as light brown sticky gum.

LCMS (ESI+): m/z 319.2 [M+H]⁺

### Step B

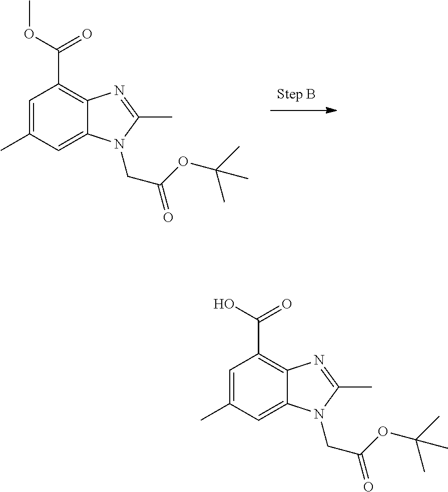 [SMILES: COC(=O)c1cc(C)cc2c1nc(C)n2CC(=O)OC(C)(C)C ; Cc1cc(C(=O)O)c2nc(C)n(CC(=O)OC(C)(C)C)c2c1]

Methyl 1-[2-(tert-butoxy)-2-oxoethyl]-2,6-dimethyl-1H-1,3-benzodiazole-4-carboxylate (50.0 mg, 0.157 mmol) was dissolved in MeCN (1.6 mL) and LiBr (272.8 mg, 3.141 mmol) was added as an aqueous solution (0.4 mL), followed by Et₃N (0.218 mL, 1.570 mmol). Reaction was stirred for 96 h. Solvents were evaporated, resulting residue dissolved in DMSO and purified with reverse phase flash chromatography (C18, H₂O:MeCN+0.1% FA) to give 1-[2-(tert-butoxy)-2-oxoethyl]-2,6-dimethyl-1H-1,3-benzodiazole-4-carboxylic acid (27.5 mg, 0.090 mmol, 57.5%) as white powder.

LCMS (ESI+): m/z 305.1 [M+H]⁺

### Step C

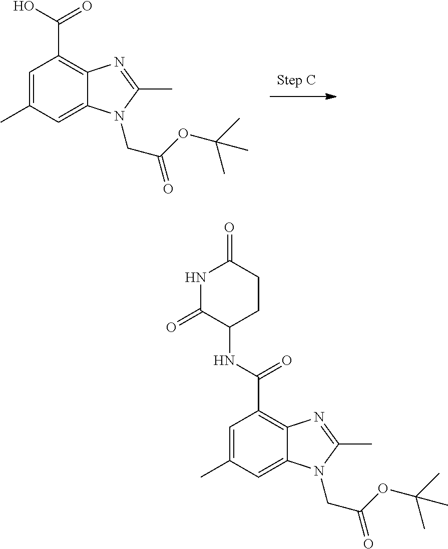 [SMILES: Cc1cc(C(=O)O)c2nc(C)n(CC(=O)OC(C)(C)C)c2c1 ; Cc1cc(C(=O)NC2CCC(=O)NC2=O)c2nc(C)n(CC(=O)OC(C)(C)C)c2c1]

1-[2-(tert-Butoxy)-2-oxoethyl]-2,6-dimethyl-1H-1,3-benzodiazole-4-carboxylic acid (27.5 mg, 0.090 mmol) and 3-aminopiperidine-2,6-dione hydrochloride (22.3 mg, 0.136 mmol) were dissolved in dry DMF (1.0 mL) and DIPEA (0.047 mL, 0.271 mmol) was added. To it, HATU (41.2 mg, 0.108 mmol) was added as a solution in DMF (0.8 mL) and the reaction was stirred at room temperature under argon atmosphere. After 1 h complete consumption of the starting material was noted. DMF was evaporated and resulting residue dissolved in DMSO. The solution was purified with reverse phase flash chromatography (C18, H₂O:MeCN+0.1% FA) to give tert-butyl 2-{4-[(2,6-dioxopiperidin-3-yl)carbamoyl]-2,6-dimethyl-1H-1,3-benzodiazol-1-yl}acetate (20.6 mg, 0.050 mmol, 55.0%) as white solid.

LCMS (ESI+): m/z 415.5 [M+H]⁺

### Step D

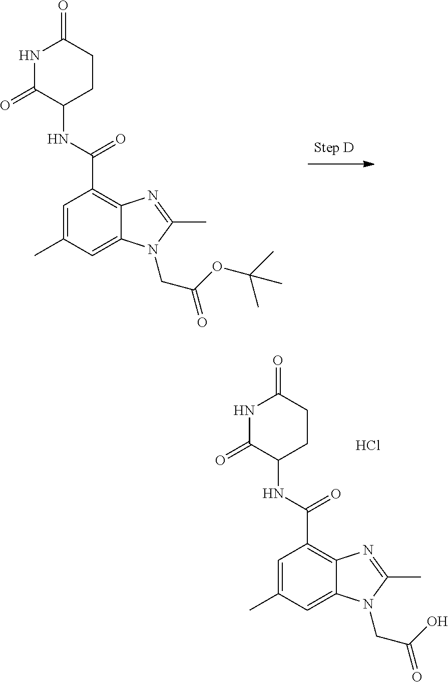 [SMILES: Cc1cc(C(=O)NC2CCC(=O)NC2=O)c2nc(C)n(CC(=O)OC(C)(C)C)c2c1 ; Cc1cc(C(=O)NC2CCC(=O)NC2=O)c2nc(C)n(CC(=O)O)c2c1 ; Cl]

tert-Butyl 2-{4-[(2,6-dioxopiperidin-3-yl)carbamoyl]-2,6-dimethyl-1H-1,3-benzodiazol-1-yl}acetate (20.6 mg, 0.050 mmol) was dissolved in DCM (0.381 mL) under argon atmosphere and TFA (0.381 mL, 4.970 mmol) was added. Reaction was stirred for 16 h at room temperature. After complete consumption of the starting material, the mixture was concentrated under reduced pressure to dryness and an aqueous solution of 1M HCl (1.0 mL, 1.000 mmol) was added. The solution was concentrated under reduced pressure and the procedure was repeated twice. The resulting residue was dissolved in water and freeze-dried to give 2-{4-[(2,6-dioxopiperidin-3-yl)carbamoyl]-2,6-dimethyl-1H-1,3-benzodiazol-1-yl}acetic acid hydrochloride (19.2 mg, 0.049 mmol, 97.6%).

LCMS (ESI+): m/z 359.05 [M+H]⁺

### Step E

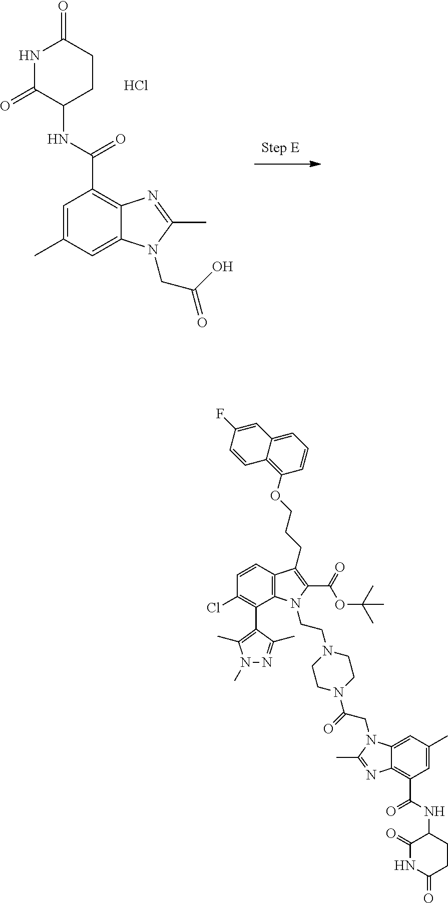 [SMILES: Cc1cc(C(=O)NC2CCC(=O)NC2=O)c2nc(C)n(CC(=O)O)c2c1 ; Cl ; Cc1cc(C(=O)NC2CCC(=O)NC2=O)c2nc(C)n(CC(=O)N3CCN(CCn4c(C(=O)OC(C)(C)C)c(CCCOc5cccc6cc(F)ccc56)c5ccc(Cl)c(-c6c(C)nn(C)c6C)c54)CC3)c2c1]

tert-Butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (22.5 mg, 0.033 mmol) and 2-{4-[(2,6-dioxopiperidin-3-yl)carbamoyl]-2,6-dimethyl-1H-1,3-benzodiazol-1-yl}acetic acid hydrochloride (14.5 mg, 0.037 mmol) were dissolved in dry DMF (1.0 mL) under argon atmosphere. To it, DIPEA (0.029 mL, 0.167 mmol) was added, followed by HATU (13.3 mg, 0.035 mmol) as a solution in DMF (0.7 mL). Reaction was stirred at room temperature for 30 min. After complete consumption of the starting material, the solution was diluted with DCM and poured into brine, separated and then washed with aqueous NaHCO₃ (saturated), again brine and water. Then, organic layer was collected, dried over anhydrous MgSO₄ and concentrated under reduced pressure to give crude tert-butyl 6-chloro-1-{2-[4-(2-{4-[(2,6-dioxopiperidin-3-yl)carbamoyl]-2,6-dimethyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (40.0 mg) that was used in the next step without further purification.

LCMS (ESI+): m/z 1014.5 [M+H]⁺

### Step F

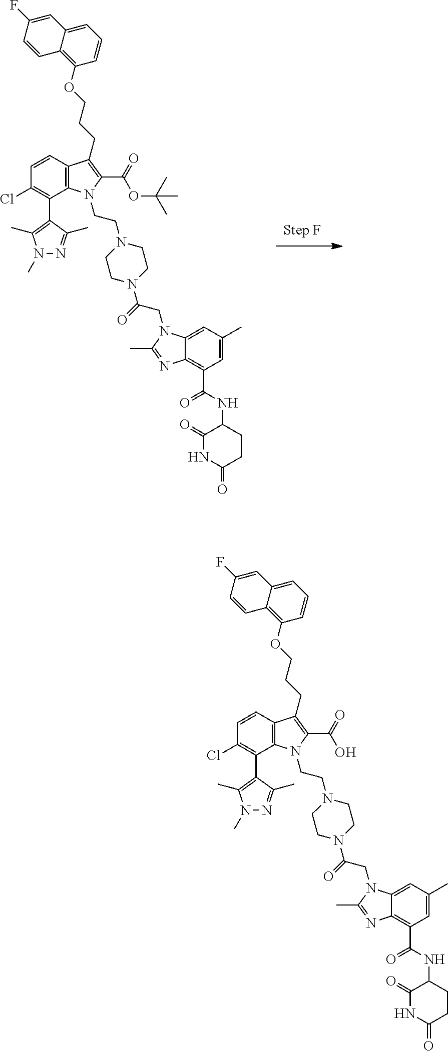 [SMILES: Cc1cc(C(=O)NC2CCC(=O)NC2=O)c2nc(C)n(CC(=O)N3CCN(CCn4c(C(=O)OC(C)(C)C)c(CCCOc5cccc6cc(F)ccc56)c5ccc(Cl)c(-c6c(C)nn(C)c6C)c54)CC3)c2c1 ; Cc1cc(C(=O)NC2CCC(=O)NC2=O)c2nc(C)n(CC(=O)N3CCN(CCn4c(C(=O)O)c(CCCOc5cccc6cc(F)ccc56)c5ccc(Cl)c(-c6c(C)nn(C)c6C)c54)CC3)c2c1]

tert-Butyl 6-chloro-1-{2-[4-(2-{4-[(2,6-dioxopiperidin-3-yl)carbamoyl]-2,6-dimethyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (40.9 mg, crude) was dissolved in dry DCM (0.40 mL) under argon atmosphere and TFA (0.40 mL) was added. The reaction was stirred at room temperature for 16 h. After complete consumption of the starting material, the solution was concentrated with a stream of argon and further dried under reduced pressure. The resulting residue was dissolved in DMSO and purified with preparative HPLC (C18, H₂O:MeCN+0.1% FA) to give 6-chloro-1-(2-(4-(2-(4-((2,6-dioxopiperidin-3-yl)carbamoyl)-2,6-dimethyl-1H-benzo[d]imidazol-1-yl)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (13.3 mg, 0.014 mmol, 42.4% over 2 steps) as off white solid.

LCMS (ESI+): m/z 958.3 [M+H]⁺

¹H NMR (500 MHz, DMSO, 353 K) δ 10.55 (s, 1H), 10.12 (d, J=7.2 Hz, 1H), 8.25 (dd, J=9.3, 5.9 Hz, 1H), 7.72 (d, J=8.5 Hz, 1H), 7.68 (dd, J=1.6, 0.7 Hz, 1H), 7.59 (dd, J=10.4, 2.6 Hz, 1H), 7.43 (s, 1H), 7.43-7.42 (m, 1H), 7.40-7.38 (m, 1H), 7.33 (ddd, J=9.3, 8.6, 2.6 Hz, 1H), 7.23 (d, J=8.5 Hz, 1H), 6.88 (dd, J=5.3, 3.4 Hz, 1H), 5.16 (s, 2H), 4.85 (ddd, J=12.3, 7.3, 5.2 Hz, 1H), 4.34-4.19 (m, 4H), 3.78 (s, 3H), 3.53-3.37 (m, 4H), 3.33-3.26 (m, 2H), 2.85-2.76 (m, 1H), 2.63-2.54 (m, 2H), 2.47-2.46 (m, 6H), 2.33-2.08 (m, 8H), 2.08-1.98 (m, 4H), 1.91 (s, 3H).

### Example 121. 1-{2-[4-(2-{6-Amino-4-[(2,6-dioxopiperidin-3-yl)carbamoyl]-2-methyl-1H-1,3-benzodiazol-1-yl}acetyl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylic acid (275)

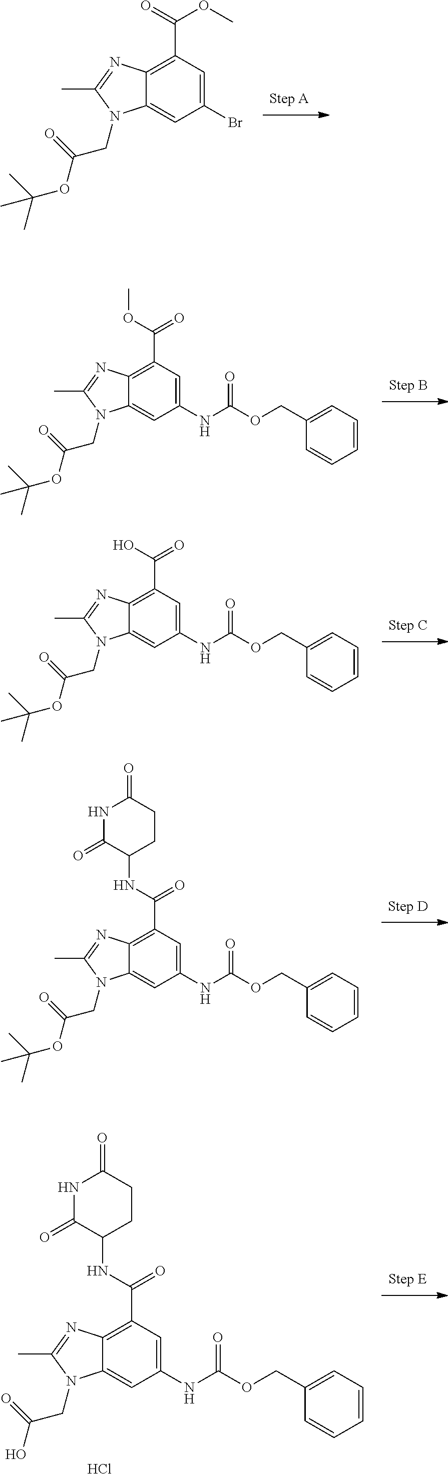 [SMILES: COC(=O)c1cc(Br)cc2c1nc(C)n2CC(=O)OC(C)(C)C ; COC(=O)c1cc(NC(=O)OCc2ccccc2)cc2c1nc(C)n2CC(=O)OC(C)(C)C ; Cc1nc2c(C(=O)O)cc(NC(=O)OCc3ccccc3)cc2n1CC(=O)OC(C)(C)C ; Cc1nc2c(C(=O)NC3CCC(=O)NC3=O)cc(NC(=O)OCc3ccccc3)cc2n1CC(=O)OC(C)(C)C ; Cc1nc2c(C(=O)NC3CCC(=O)NC3=O)cc(NC(=O)OCc3ccccc3)cc2n1CC(=O)O ; Cl]

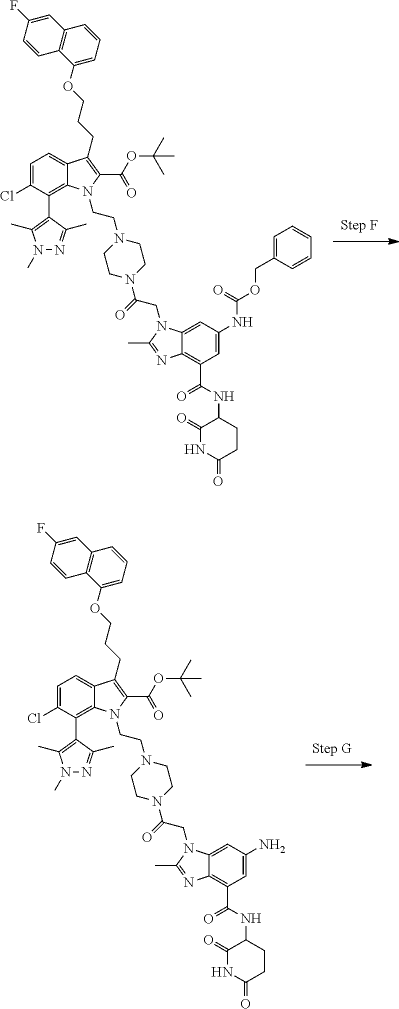 [SMILES: Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)cc(NC(=O)OCc6ccccc6)cc54)CC3)c12 ; Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)cc(N)cc54)CC3)c12]

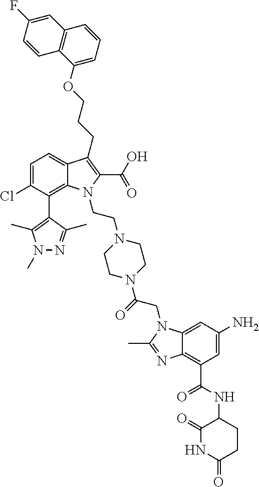 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cc(N)cc54)CC3)c12]

### Step A

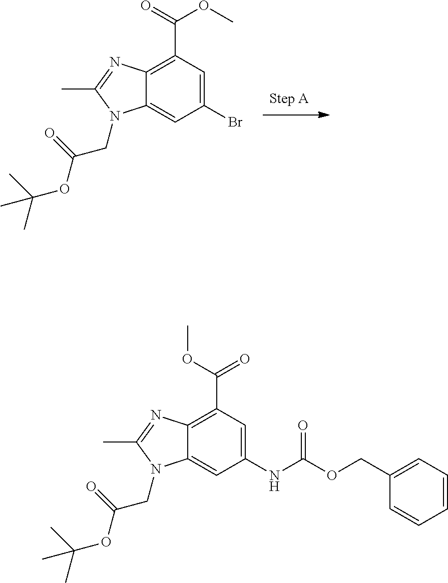 [SMILES: COC(=O)c1cc(Br)cc2c1nc(C)n2CC(=O)OC(C)(C)C ; COC(=O)c1cc(NC(=O)OCc2ccccc2)cc2c1nc(C)n2CC(=O)OC(C)(C)C]

A stirred solution of methyl 6-bromo-1-(2-(tert-butoxy)-2-oxoethyl)-2-methyl-1H-benzo[d]imidazole-4-carboxylate (4.0 g, 10.47 mmol), benzyl carbamate and Cs₂CO₃ (5.11 g, 15.70 mmol) in 1,4-dioxane (120 mL) was deoxygenated with argon for 5 min. Pd₂(dba)₃ (0.96 g, 1.05 mmol) and XPhos (0.75 g, 1.57 mmol) was then added to the reaction mixture and the reaction vessel was again deoxygenated with argon for 10 min. The reaction mixture was then stirred for 16 h at 90° C. After complete consumption of the starting material (monitored by LCMS) the reaction mixture was cooled to room temperature and filtered through Celite®. The filtrate was concentrated under reduced pressure. Crude material was purified by flash column chromatography (SiO₂, 80% EtOAc in hexane) to afford methyl 6-{[(benzyloxy)carbonyl]amino}-1-[2-(tert-butoxy)-2-oxoethyl]-2-methyl-1H-1,3-benzodiazole-4-carboxylate (2.5 g, 5.51 mmol, 52%) as off white solid.

LCMS (ESI+): m/z 454.3 [M+H]⁺

### Step B

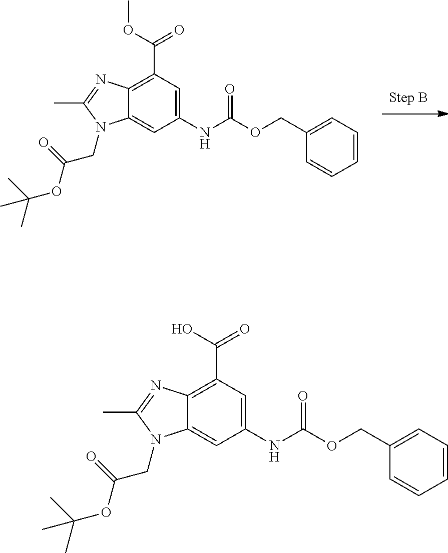 [SMILES: COC(=O)c1cc(NC(=O)OCc2ccccc2)cc2c1nc(C)n2CC(=O)OC(C)(C)C ; Cc1nc2c(C(=O)O)cc(NC(=O)OCc3ccccc3)cc2n1CC(=O)OC(C)(C)C]

To a solution of methyl 6-{[(benzyloxy)carbonyl]amino}-1-[2-(tert-butoxy)-2-oxoethyl]-2-methyl-1H-1,3-benzodiazole-4-carboxylate (200.0 mg, 0.441 mmol) in H₂O (1.0 mL) and MeCN (5.0 mL) was added LiBr (766.0 mg, 8.820 mmol) and Et₃N (0.613 mL, 4.410 mmol). The mixture was stirred for 3 days at RT. The crude was concentrated in vacuo and purified by reverse phase flash chromatography (C18, H₂O:MeCN+0.1% FA) to get 6-{[(benzyloxy)carbonyl]amino}-1-[2-(tert-butoxy)-2-oxoethyl]-2-methyl-1H-1,3-benzodiazole-4-carboxylic acid (113.0 mg, 0.257 mmol, 58.3%) as white solid.

LCMS (ESI+): m/z 440.15 [M+H]⁺

### Step C

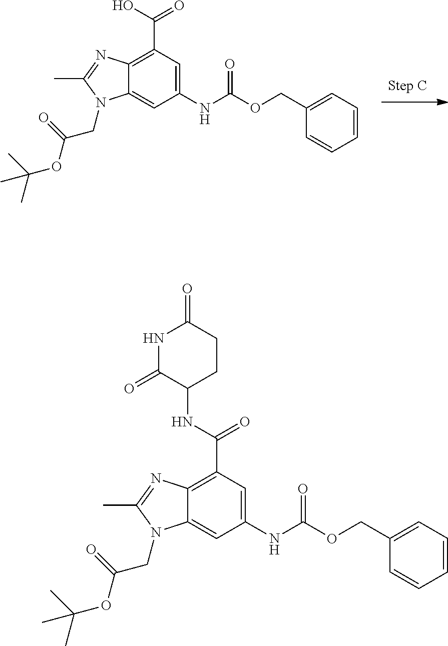 [SMILES: Cc1nc2c(C(=O)O)cc(NC(=O)OCc3ccccc3)cc2n1CC(=O)OC(C)(C)C ; Cc1nc2c(C(=O)NC3CCC(=O)NC3=O)cc(NC(=O)OCc3ccccc3)cc2n1CC(=O)OC(C)(C)C]

To a solution of 6-{[(benzyloxy)carbonyl]amino}-1-[2-(tert-butoxy)-2-oxoethyl]-2-methyl-1H-1,3-benzodiazole-4-carboxylic acid (113.0 mg, 0.257 mmol), 3-aminopiperidine-2,6-dione hydrochloride (50.8 mg, 0.309 mmol) and HATU (195.5 mg, 0.514 mmol) in dry DMF (2.0 mL) was added DIPEA (0.224 mL, 1.286 mmol). The mixture was stirred at room temperature for 30 min. The crude was purified by reverse phase flash chromatography (C18, H₂O:MeCN+0.1% FA) to obtain tert-butyl 2-(6-{[(benzyloxy)carbonyl]amino}-4-[(2,6-dioxopiperidin-3-yl)carbamoyl]-2-methyl-1H-1,3-benzodiazol-1-yl)acetate (87.0 mg, 0.158 mmol, 61.6%) as a white solid.

LCMS (ESI+): m/z 550.1 [M+H]⁺

### Step D

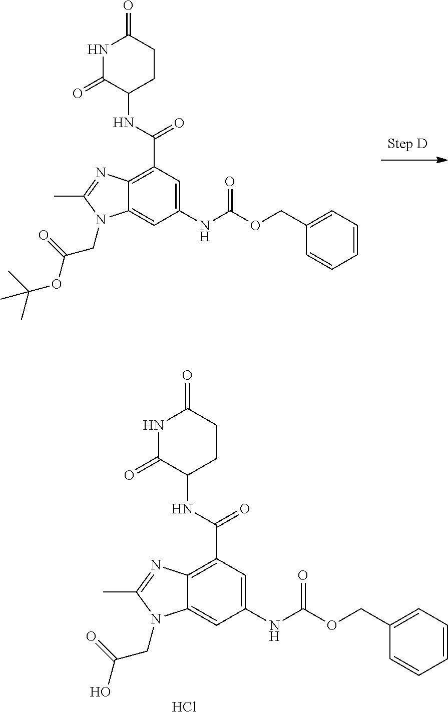 [SMILES: Cc1nc2c(C(=O)NC3CCC(=O)NC3=O)cc(NC(=O)OCc3ccccc3)cc2n1CC(=O)OC(C)(C)C ; Cc1nc2c(C(=O)NC3CCC(=O)NC3=O)cc(NC(=O)OCc3ccccc3)cc2n1CC(=O)O ; Cl]

tert-Butyl 2-(6-{[(benzyloxy)carbonyl]amino}-4-[(2,6-dioxopiperidin-3-yl)carbamoyl]-2-methyl-1H-1,3-benzodiazol-1-yl)acetate (72.0 mg, 0.131 mmol) was dissolved in DCM (1.0 mL) and to the mixture was added TFA (1.0 mL, 13.059 mmol). The reaction was stirred for 18 h at RT. The crude was concentrated in vacuo and To the resulting residue was added aqueous 1M HCl (3 mL) in water and evaporated under reduced pressure. The product 2-(6-{[(benzyloxy)carbonyl]amino}-4-[(2,6-dioxopiperidin-3-yl)carbamoyl]-2-methyl-1H-1,3-benzodiazol-1-yl)acetic acid hydrochloride (62.0 mg, 0.117 mmol, 89.3%) was obtained as off-white solid.

LCMS (ESI+): m/z 494.05 [M+H]⁺

### Step E

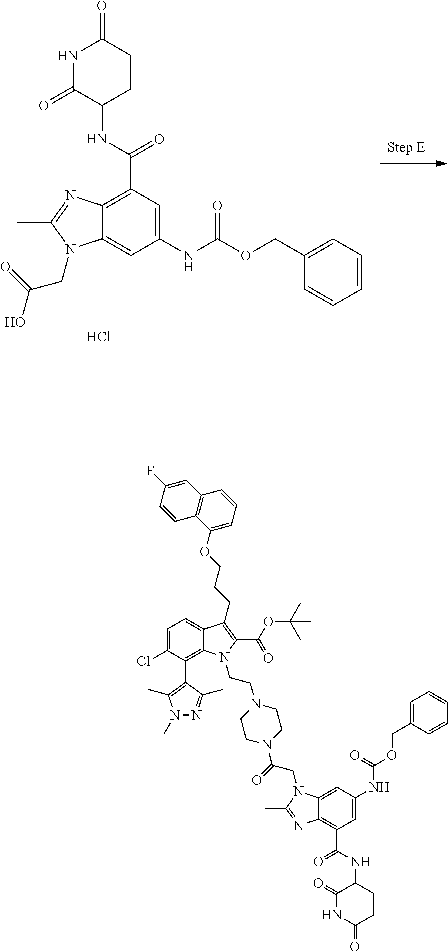 [SMILES: Cc1nc2c(C(=O)NC3CCC(=O)NC3=O)cc(NC(=O)OCc3ccccc3)cc2n1CC(=O)O ; Cl ; Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)cc(NC(=O)OCc6ccccc6)cc54)CC3)c12]

To a solution of tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (30.0 mg, 0.044 mmol), 2-(6-{[(benzyloxy)carbonyl]amino}-4-[(2,6-dioxopiperidin-3-yl)carbamoyl]-2-methyl-1H-1,3-benzodiazol-1-yl)acetic acid hydrochloride (28.3 mg, 0.053 mmol) and HATU (33.8 mg, 0.089 mmol) in dry DMF (2.0 mL) was added DIPEA (0.023 mL, 0.133 mmol) and the mixture was stirred at room temperature for 30 min. The crude was diluted with DCM and washed with brine. The organic layer was dried over Na₂SO₄, filtered and concentrated in vacuo to get 58 mg of crude tert-butyl 1-(2-{4-[2-(6-{[(benzyloxy)carbonyl]amino}-4-[(2,6-dioxopiperidin-3-yl)carbamoyl]-2-methyl-1H-1,3-benzodiazol-1-yl)acetyl]piperazin-1-yl}ethyl)-6-chloro-3-{3-[(6-fluoronaphthalen-1-yl)oxy]propyl}-7-(1,3,5-trimethyl-1H-pyrazol-4-yl)-1H-indole-2-carboxylate as yellow oil which was used in the next step without further purification.

LCMS (ESI+): m/z 1149.35 [M+H]⁺

### Step F

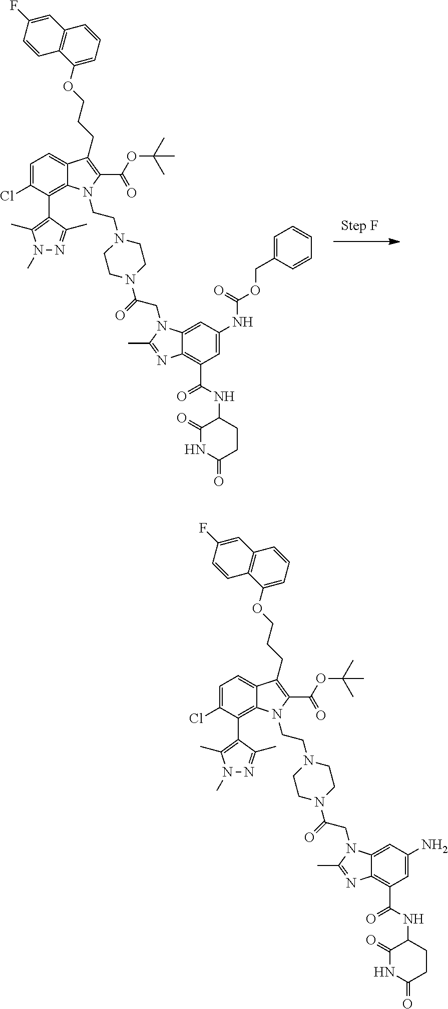 [SMILES: Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)cc(NC(=O)OCc6ccccc6)cc54)CC3)c12 ; Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)cc(N)cc54)CC3)c12]

To a solution of tert-butyl 1-(2-{4-[2-(6-{[(benzyloxy)carbonyl]amino}-4-[(2,6-dioxopiperidin-3-yl)carbamoyl]-2-methyl-1H-1,3-benzodiazol-1-yl)acetyl]piperazin-1-yl}ethyl)-6-chloro-3-{3-[(6-fluoronaphthalen-1-yl)oxy]propyl}-7-(1,3,5-trimethyl-1H-pyrazol-4-yl)-1H-indole-2-carboxylate (58 mg, crude) in EtOH (2.0 mL) and EtOAc (2.0 mL) was added Pd/C (10.0 mg) and the reaction mixture was degassed with argon for 15 min. Hydrogen was bubbled then through reaction mixture for 4 days at RT. The reaction mixture was filtered through Celite® pad and concentrated under reduced pressure to obtain crude tert-butyl 1-{2-[4-(2-{6-amino-4-[(2,6-dioxopiperidin-3-yl)carbamoyl]-2-methyl-1H-1,3-benzodiazol-1-yl}acetyl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylate (50 mg, crude), which was used in the next step without further purification.

LCMS (ESI+): m/z 1015.3 [M+H]⁺

### Step G

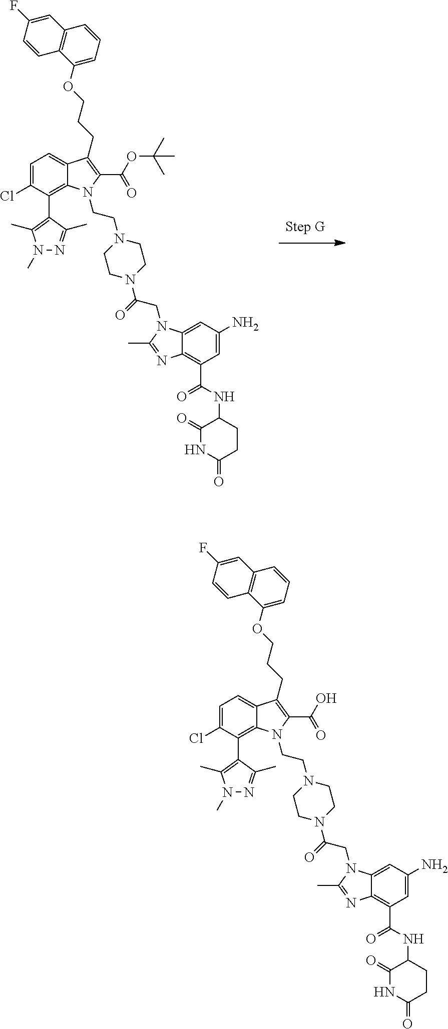 [SMILES: Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)cc(N)cc54)CC3)c12 ; Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cc(N)cc54)CC3)c12]

To a solution of tert-butyl 1-{2-[4-(2-{6-amino-4-[(2,6-dioxopiperidin-3-yl)carbamoyl]-2-methyl-1H-1,3-benzodiazol-1-yl}acetyl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylate (50 mg, crude) in DCM (1.0 mL) was added TFA (1.0 mL, 13.059 mmol). The mixture was stirred for 18 h at RT. The crude was concentrated in vacuo and purified with preparative HPLC (C18, H₂O:MeCN+0.1% FA) to obtain 1-{2-[4-(2-{6-amino-4-[(2,6-dioxopiperidin-3-yl)carbamoyl]-2-methyl-1H-1,3-benzodiazol-1-yl}acetyl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylic acid (6.5 mg, 0.007 mmol, 15.9% over 3 steps) as a white solid.

LCMS (ESI+): m/z 959.35 [M+H]⁺

¹H NMR (500 MHz, DMSO) δ 10.54 (s, 1H), 10.12 (d, J=7.3 Hz, 1H), 8.25 (dd, J=9.3, 5.9 Hz, 1H), 7.70 (d, J=8.5 Hz, 1H), 7.59 (dd, J=10.4, 2.6 Hz, 1H), 7.44-7.42 (m, 2H), 7.33 (td, J=8.9, 2.6 Hz, 1H), 7.24 (d, J=2.1 Hz, 1H), 7.21 (d, J=8.5 Hz, 1H), 6.88 (dd, J=5.6, 3.1 Hz, 1H), 6.66 (d, J=2.1 Hz, 1H), 5.00 (s, 2H), 4.82 (ddd, J=12.3, 7.3, 5.2 Hz, 1H), 4.34-4.28 (m, 1H), 4.27-4.19 (m, 3H), 3.78 (s, 3H), 3.44 (s, 4H), 3.32-3.26 (m, 2H), 2.85-2.75 (m, 1H), 2.65-2.57 (m, 1H), 2.39 (s, 3H), 2.31-2.00 (m, 15H), 1.91 (s, 3H).

### Example 122. 6-Chloro-1-(2-(4-(2-(6-chloro-4-((2,6-dioxopiperidin-3-yl)carbamoyl)-2-methyl-1H-benzo[d]imidazol-1-yl)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (276)

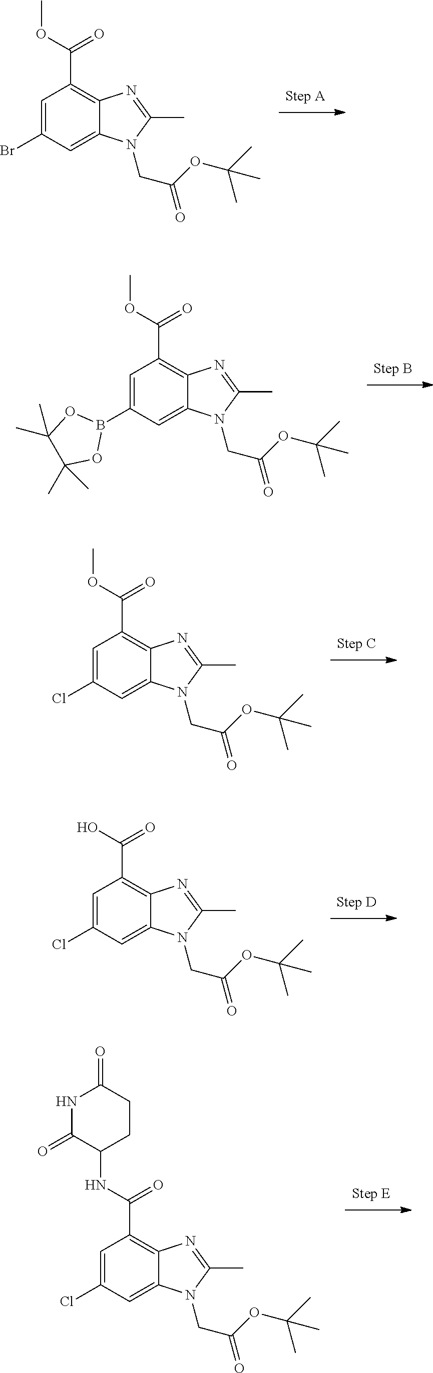

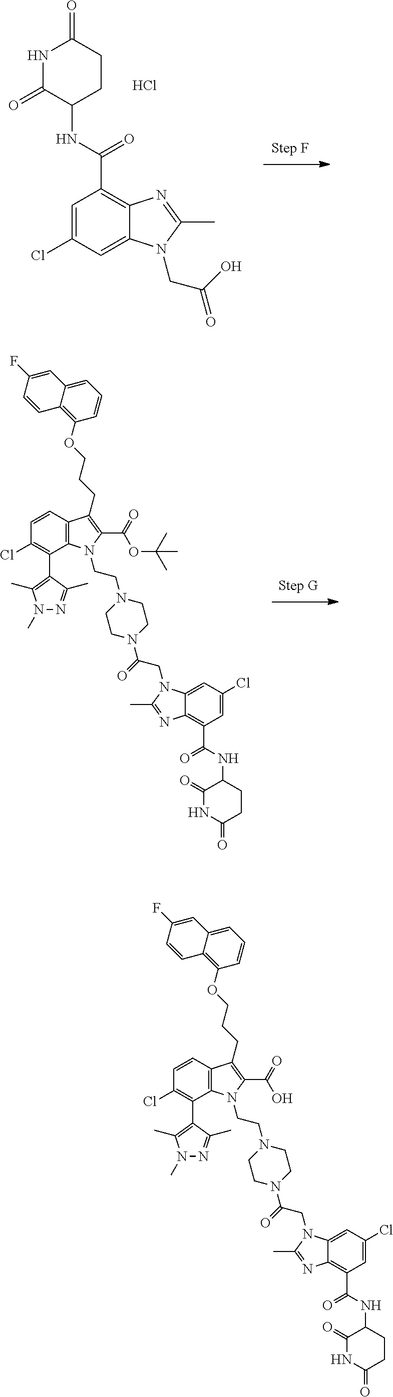 [SMILES: Cc1nc2c(C(=O)NC3CCC(=O)NC3=O)cc(Cl)cc2n1CC(=O)O ; Cl ; Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)cc(Cl)cc54)CC3)c12 ; Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cc(Cl)cc54)CC3)c12]

### Step A

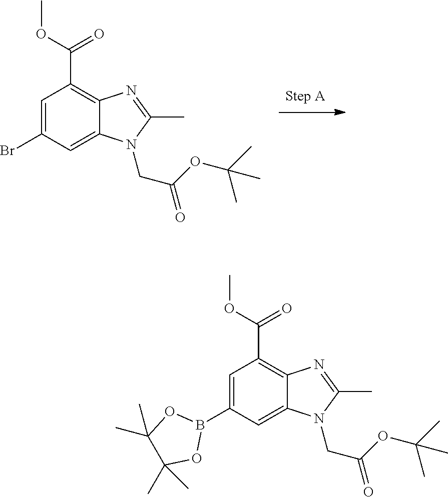 [SMILES: COC(=O)c1cc(Br)cc2c1nc(C)n2CC(=O)OC(C)(C)C ; COC(=O)c1cc(B2OC(C)(C)C(C)(C)O2)cc2c1nc(C)n2CC(=O)OC(C)(C)C]

A stirred suspension of methyl 6-bromo-1-(2-(tert-butoxy)-2-oxoethyl)-2-methyl-1H-benzo[d]imidazole-4-carboxylate (2.0 g, 5.22 mmol), BISPIN (1.46 g, 5.74 mmol) and potassium acetate (1.54 g, 15.67 mmol) in 1,4-dioxane (45 mL) was sparged with argon for 10 min. Pd(dppf)Cl2·DCM (0.213 g, 0.261 mmol) was added to the reaction mixture and again sparged with argon for 10 min. Finally, the reaction mixture was stirred for 15 h at 90° C. in a sealed tube. After complete consumption of the starting material, the reaction mixture was cooled to room temperature and filtered through Celite® pad. The filtrate was concentrated under reduced pressure. Crude material was purified by column chromatography (SiO₂, 80% EtOAc in hexane) to afford 1.5 g of methyl 1-(2-(tert-butoxy)-2-oxoethyl)-2-methyl-6-(4,4,5,5-tetramethyl-1,3,2-dioxaborolan-2-yl)-1H-benzo[d]imidazole-4-carboxylate (1.50 g, 3.48 mmol, 66%) as off white solid.

LCMS (ESI+): m/z 430.6 [M+H]⁺

### Step B

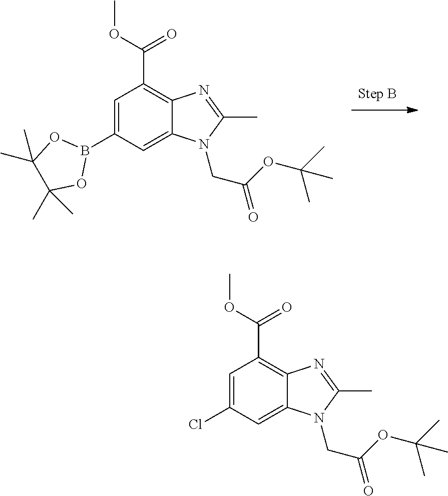 [SMILES: COC(=O)c1cc(B2OC(C)(C)C(C)(C)O2)cc2c1nc(C)n2CC(=O)OC(C)(C)C ; COC(=O)c1cc(Cl)cc2c1nc(C)n2CC(=O)OC(C)(C)C]

To a stirring solution of methyl 1-(2-(tert-butoxy)-2-oxoethyl)-2-methyl-6-(4,4,5,5-tetramethyl-1,3,2-dioxaborolan-2-yl)-1H-benzo[d]imidazole-4-carboxylate (2.2 g, 5.1 mmol) in a mixture of MeOH and H₂O (2:1) (90 mL), was added CuCl₂ (2.06 g, 15.35 mmol) at room temperature. The reaction mixture was then stirred for 4 h at 60° C. After complete consumption of the starting material, the volatiles were evaporated under reduced pressure and the residue was then dissolved in water and extracted by DCM. The combined organic layer was dried over anhydrous Na₂SO₄, concentrated under reduced pressure to get the crude material which was purified by column chromatography (SiO₂, 80% EtOAc in hexane) to afford methyl 1-(2-(tert-butoxy)-2-oxoethyl)-6-chloro-2-methyl-1H-benzo[d]imidazole-4-carboxylate (0.95 g, 2.8 mmol, 55%) as light brown sticky gum.

LCMS (ESI+): m/z 338.8 [M+H]⁺

### Step C

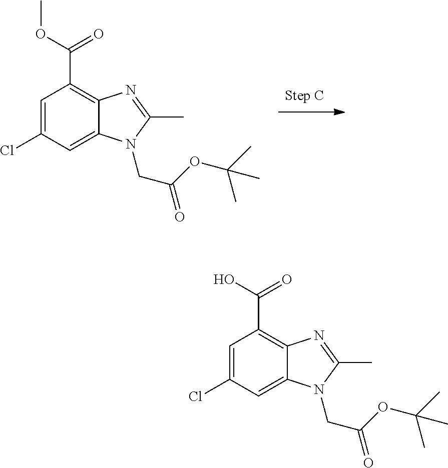 [SMILES: COC(=O)c1cc(Cl)cc2c1nc(C)n2CC(=O)OC(C)(C)C ; Cc1nc2c(C(=O)O)cc(Cl)cc2n1CC(=O)OC(C)(C)C]

To a solution of methyl 1-[2-(tert-butoxy)-2-oxoethyl]-6-chloro-2-methyl-1H-1,3-benzodiazole-4-carboxylate (100.0 mg, 0.295 mmol) in H₂O (1.0 mL) and MeCN (5.0 mL) was added LiBr (512.6 mg, 5.903 mmol) and Et₃N (0.411 mL, 2.952 mmol). The mixture was stirred for 3 days at RT. The crude was concentrated in vacuum and purified by reverse phase flash chromatography (C18, H₂O:MeCN+0.1% FA) to get a 1-[2-(tert-butoxy)-2-oxoethyl]-6-chloro-2-methyl-1H-1,3-benzodiazole-4-carboxylic acid (85.9 mg, 0.265 mmol, 89.6%) as a white solid.

LCMS (ESI+): m/z 325.1 [M+H]⁺

### Step D

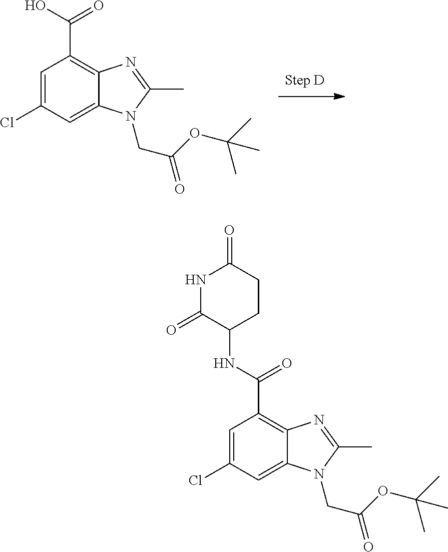 [SMILES: Cc1nc2c(C(=O)O)cc(Cl)cc2n1CC(=O)OC(C)(C)C ; Cc1nc2c(C(=O)NC3CCC(=O)NC3=O)cc(Cl)cc2n1CC(=O)OC(C)(C)C]

1-[2-(tert-Butoxy)-2-oxoethyl]-6-chloro-2-methyl-1H-1,3-benzodiazole-4-carboxylic acid (85.9 mg, 0.265 mmol) was dissolved in dry DMF (2.6 mL) under argon atmosphere. To it, DIPEA (0.138 mL, 0.794 mmol), followed by HATU (120.7 mg, 0.317 mmol) was added and the reaction was stirred for 15 min at room temperature. Next, 3-aminopiperidine-2,6-dione hydrochloride (65.3 mg, 0.397 mmol) was added and the reaction was continued for 20 min. After complete consumption of the starting material, DMF was evaporated, resulting residue dissolved in DMSO (4.0 mL) and the reaction product was purified with reverse phase flash chromatography (C18, H₂O:MeCN+0.1% FA). Samples containing product were merged and MeCN evaporated, white precipitate filtered off, washed with water and dried under reduced pressure to give tert-butyl 2-{6-chloro-4-[(2,6-dioxopiperidin-3-yl)carbamoyl]-2-methyl-1H-1,3-benzodiazol-1-yl}acetate (70.1 mg, 0.161 mmol, 61.0%) as white solid.

LCMS (ESI+): m/z 435.2 [M+H]⁺

### Step E

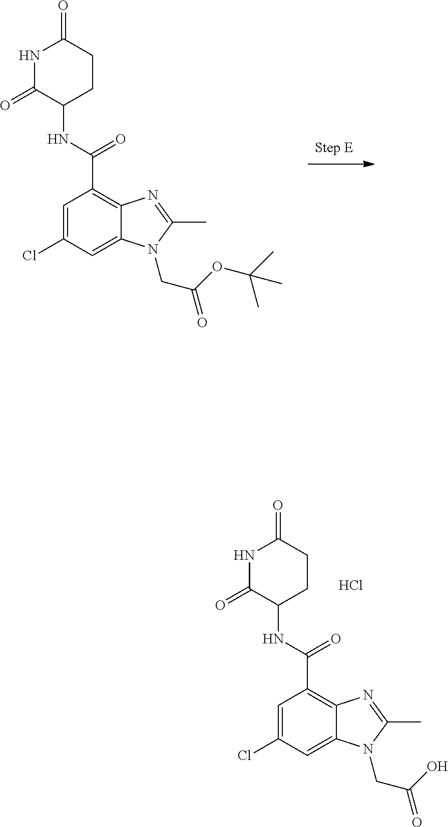 [SMILES: Cc1nc2c(C(=O)NC3CCC(=O)NC3=O)cc(Cl)cc2n1CC(=O)OC(C)(C)C ; Cc1nc2c(C(=O)NC3CCC(=O)NC3=O)cc(Cl)cc2n1CC(=O)O ; Cl]

tert-Butyl 2-{6-chloro-4-[(2,6-dioxopiperidin-3-yl)carbamoyl]-2-methyl-1H-1,3-benzodiazol-1-yl}acetate (66.0 mg, 0.152 mmol) was dissolved in dry DCM (1.2 mL) under argon atmosphere and TFA (1.2 mL, 15.177 mmol) was added. Reaction was stirred at room temperature for 16 h. After complete consumption of the starting material, the solution was concentrated with strong stream of argon and dried under reduced pressure. The resulting residue was three times dissolved in 1M aqueous solution of HCl and concentrated to dryness to give crude 2-{6-chloro-4-[(2,6-dioxopiperidin-3-yl)carbamoyl]-2-methyl-1H-1,3-benzodiazol-1-yl}acetic acid hydrochloride (61.1 mg) as white solid.

LCMS (ESI+): m/z 378.9 [M+H]⁺

### Step F

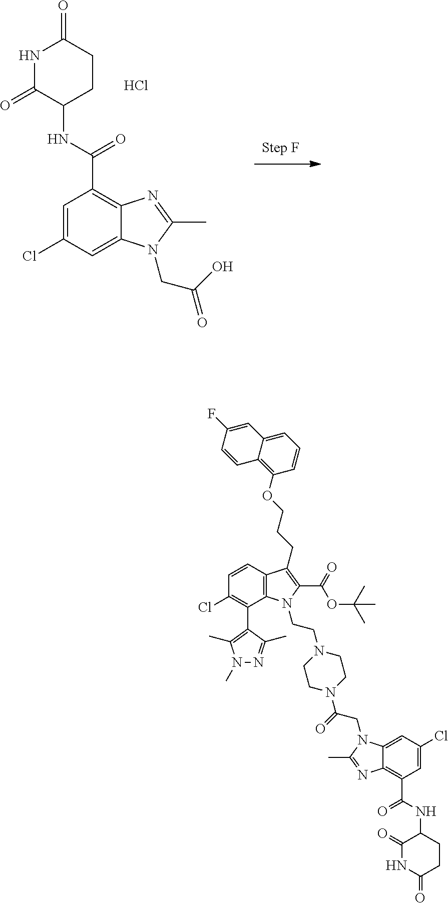 [SMILES: Cc1nc2c(C(=O)NC3CCC(=O)NC3=O)cc(Cl)cc2n1CC(=O)O ; Cl ; Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)cc(Cl)cc54)CC3)c12]

2-{6-Chloro-4-[(2,6-dioxopiperidin-3-yl)carbamoyl]-2-methyl-1H-1,3-benzodiazol-1-yl}acetic acid hydrochloride (20.3 mg, crude) was dissolved in dry DMF (2.0 mL) under argon atmosphere and DIPEA (0.039 mL, 0.222 mmol) was added. To it, tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (30 mg, 0.044 mmol) was added, followed by the dropwise addition of HATU (17.8 mg, 0.047 mmol) as a solution in DMF (2.0 mL). Reaction was stirred at room temperature under argon for 15 min. After complete consumption of the starting material, the solution was diluted with DCM and poured into brine. Organic layer was separated and washed with NaHCO₃, again brine and water. Organic layer was collected, dried over anhydrous MgSO₄, filtered and dried under reduced pressure to give tert-butyl 6-chloro-1-{2-[4-(2-{6-chloro-4-[(2,6-dioxopiperidin-3-yl)carbamoyl]-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (37.8 mg, crude) as orange oil that was used directly in the next step without further purification.

LCMS (ESI+): m/z 1034.3 [M+H]⁺

### Step G

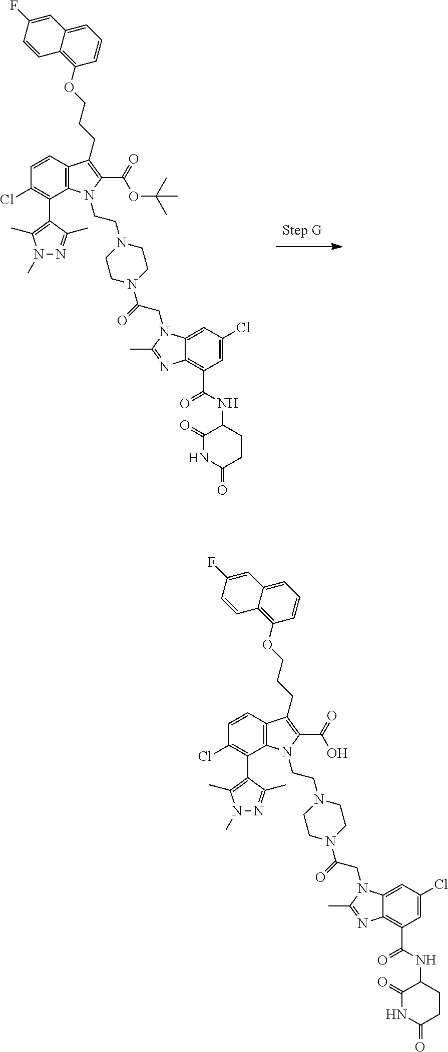 [SMILES: Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)cc(Cl)cc54)CC3)c12 ; Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cc(Cl)cc54)CC3)c12]

Crude tert-butyl 6-chloro-1-{2-[4-(2-{6-chloro-4-[(2,6-dioxopiperidin-3-yl)carbamoyl]-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (37.8 mg) was dissolved in dry DCM (0.300 mL) under argon atmosphere and TFA (0.296 mL, 3.87 mmol) was added. Reaction was stirred at room temperature for 16 h. After complete consumption of the starting material, the mixture was concentrated with a strong stream of argon and dried under vacuum. The resulting residue was dissolved in DMSO and purified with preparative HPLC (C18, H₂O:MeCN+0.1% FA) to give 6-chloro-1-(2-(4-(2-(6-chloro-4-((2,6-dioxopiperidin-3-yl)carbamoyl)-2-methyl-1H-benzo[d]imidazol-1-yl)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (19.7 mg, 0.020 mmol, 45% over 2 steps) as white powder.

LCMS (ESI+): m/z 978.20 [M+H]⁺

¹H NMR (500 MHz, DMSO, 353 K) δ 10.61 (s, 1H), 10.07 (d, J=7.2 Hz, 1H), 8.27 (dd, J=9.3, 5.9 Hz, 1H), 7.85-7.77 (m, 2H), 7.74 (d, J=8.5 Hz, 1H), 7.61 (dd, J=10.4, 2.6 Hz, 1H), 7.51-7.40 (m, 2H), 7.34 (td, J=8.9, 2.6 Hz, 1H), 7.24 (d, J=8.5 Hz, 1H), 6.90 (dd, J=5.3, 3.4 Hz, 1H), 5.25 (s, 2H), 4.88 (ddd, J=12.4, 7.2, 5.2 Hz, 1H), 4.38-4.18 (m, 4H), 3.80 (s, 3H), 3.57-3.36 (m, 4H), 3.31 (dd, J=8.5, 6.5 Hz, 2H), 2.83 (ddd, J=17.3, 13.1, 5.5 Hz, 1H), 2.67-2.60 (m, 1H), 2.51 (s, 3H), 2.37-2.09 (m, 10H), 2.05 (s, 3H), 1.93 (s, 3H).

### Example 123. 6-Chloro-1-{2-[4-(2-{4-[(2,6-dioxopiperidin-3-yl)carbamoyl]-6-methoxy-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (277)

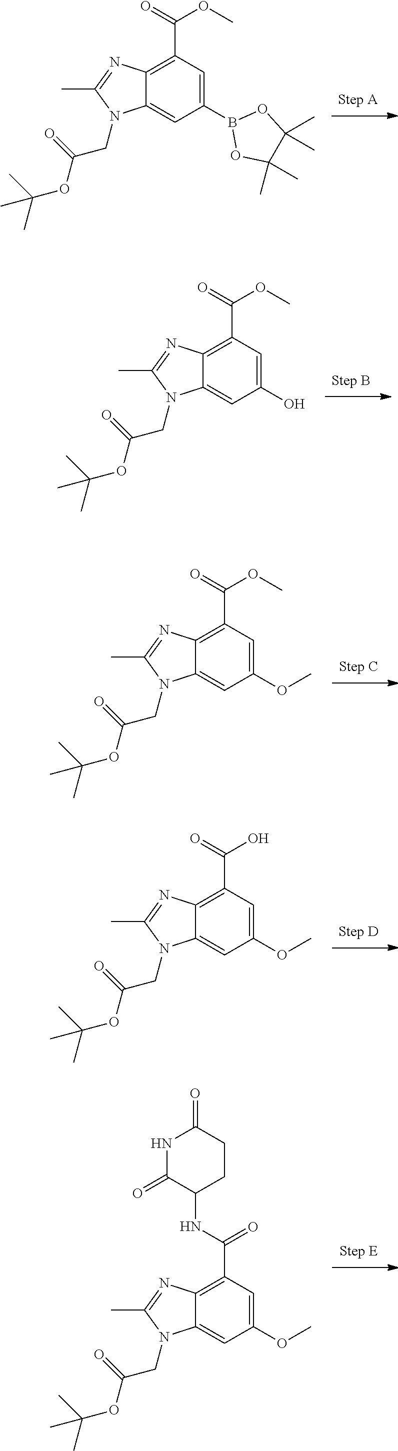

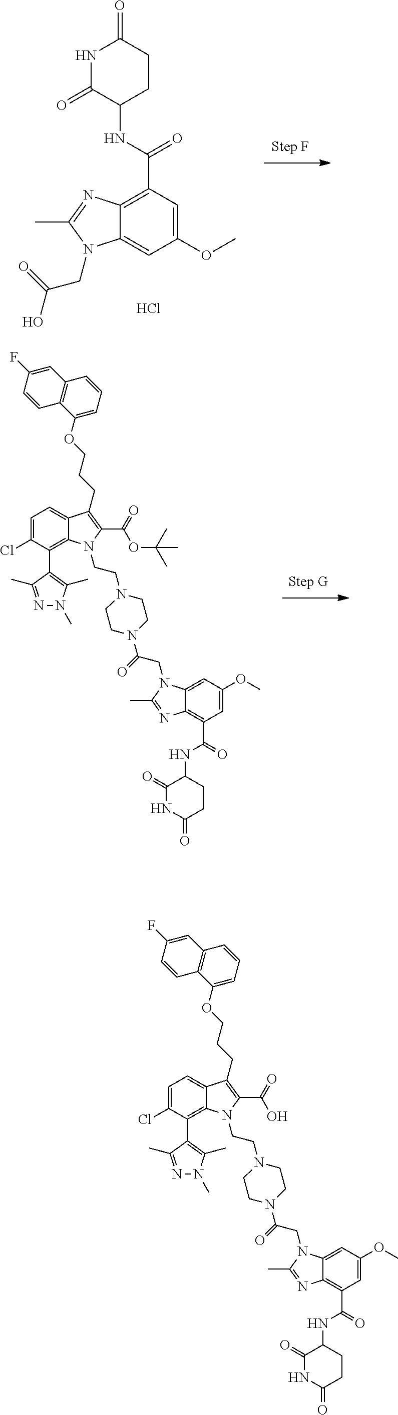

### Step A

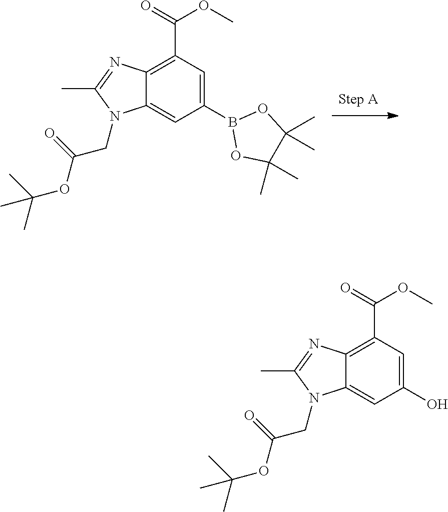

To a stirring solution of methyl 1-(2-(tert-butoxy)-2-oxoethyl)-2-methyl-6-(4,4,5,5-tetramethyl-1,3,2-dioxaborolan-2-yl)-1H-benzo[d]imidazole-4-carboxylate (1.5 g, 3.49 mmol) in a mixture of EtOH and H₂O (2:1) (60 mL), was added mCPBA (0.78 g, 4.53 mmol). The reaction mixture was stirred for 4 h at RT. After complete consumption of the starting material (monitored by LCMS), the volatiles were evaporated under reduced pressure. The residue was then dissolved in a mixture MeOH and DCM (1:10) and washed with saturated NaHCO₃ solution. The aqueous part was then extracted with mixture MeOH and DCM (1:10) and the combined organic layer was washed with brine. The organic layer was dried over anhydrous Na₂SO₄ and concentrated under reduced pressure. Crude material was purified by flash chromatography (SiO₂, 5% MeOH in DCM) to obtain methyl 1-(2-(tert-butoxy)-2-oxoethyl)-6-hydroxy-2-methyl-1H-benzo[d]imidazole-4-carboxylate (750 mg, 2.34 mmol, 67%) as off white solid

LCMS (ESI+): m/z 321.2 [M+H]⁺

### Step B

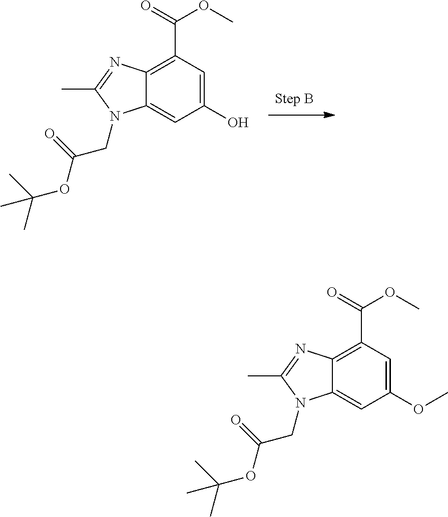 [SMILES: COC(=O)c1cc(O)cc2c1nc(C)n2CC(=O)OC(C)(C)C ; COC(=O)c1cc(OC)cc2c1nc(C)n2CC(=O)OC(C)(C)C]

To a stirring solution of methyl 1-(2-(tert-butoxy)-2-oxoethyl)-6-hydroxy-2-methyl-1H-benzo[d]imidazole-4-carboxylate (250 mg, 0.781 mmol) in DMF (7 mL) was added K₂CO₃ (323 mg, 2.34 mmol). The reaction mixture was then stirred for 1 h at RT. Iodomethane (0.243 mL, 3.91 mmol) was then added to the reaction mixture and stirred for 16 h at RT. After complete consumption of the starting material (monitored by LCMS), the reaction mixture was filtered through sintered funnel. The filtrate was diluted with EtOAc and washed twice with water and brine solution. The organic layer was dried over anhydrous Na₂SO₄ and concentrated under reduced pressure. Crude material was purified by flash chromatography (SiO₂, 5% MeOH in DCM) to obtain methyl 1-[2-(tert-butoxy)-2-oxoethyl]-6-methoxy-2-methyl-1H-1,3-benzodiazole-4-carboxylate (90 mg, 0.27 mmol, 34.5%) as off white solid.

LCMS (ESI+): m/z 335.2 [M+H]⁺

### Step C

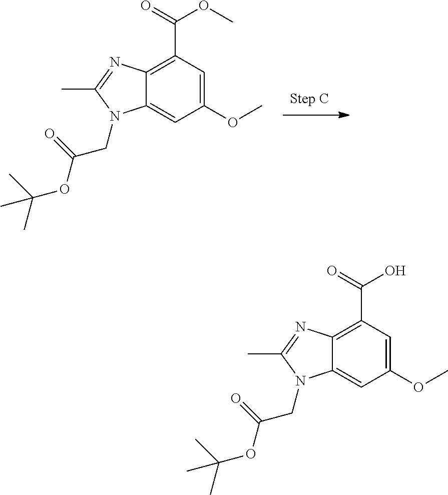 [SMILES: COC(=O)c1cc(OC)cc2c1nc(C)n2CC(=O)OC(C)(C)C ; COc1cc(C(=O)O)c2nc(C)n(CC(=O)OC(C)(C)C)c2c1]

To a solution of methyl 1-[2-(tert-butoxy)-2-oxoethyl]-6-methoxy-2-methyl-1H-1,3-benzodiazole-4-carboxylate (81.0 mg, 0.242 mmol) in H₂O (1.0 mL) and MeCN (5.0 mL) was added LiBr (420.7 mg, 4.845 mmol) and Et₃N (0.337 mL, 2.422 mmol). The mixture was stirred for 3 days at RT. The crude was concentrated in vacuum and purified by reverse phase flash chromatography (C18, H₂O:MeCN+0.1% FA) to obtain 1-[2-(tert-butoxy)-2-oxoethyl]-6-methoxy-2-methyl-1H-1,3-benzodiazole-4-carboxylic acid (73.0 mg, 0.228 mmol, 94.1%) as white solid.

LCMS (ESI+): m/z 321.0 [M+H]⁺

### Step D

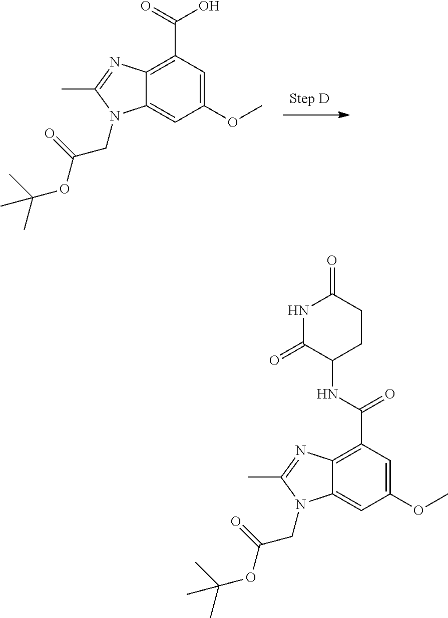 [SMILES: COc1cc(C(=O)O)c2nc(C)n(CC(=O)OC(C)(C)C)c2c1 ; COc1cc(C(=O)NC2CCC(=O)NC2=O)c2nc(C)n(CC(=O)OC(C)(C)C)c2c1]

To a solution of 1-[2-(tert-butoxy)-2-oxoethyl]-6-methoxy-2-methyl-1H-1,3-benzodiazole-4-carboxylic acid (73.0 mg, 0.228 mmol), 3-aminopiperidine-2,6-dione hydrochloride (45.0 mg, 0.273 mmol) and HATU (173.3 mg, 0.456 mmol) in dry DMF (2.0 mL) was added DIPEA (0.119 mL, 0.684 mmol). The mixture was stirred at room temperature for 30 min. The crude was purified by reverse phase flash chromatography (C18, H₂O:MeCN+0.1% FA) to obtain tert-butyl 2-{4-[(2,6-dioxopiperidin-3-yl)carbamoyl]-6-methoxy-2-methyl-1H-1,3-benzodiazol-1-yl}acetate (63.0 mg, 0.146 mmol, 64.2%) as a white solid.

LCMS (ESI+): m/z 431.15 [M+H]⁺

### Step E

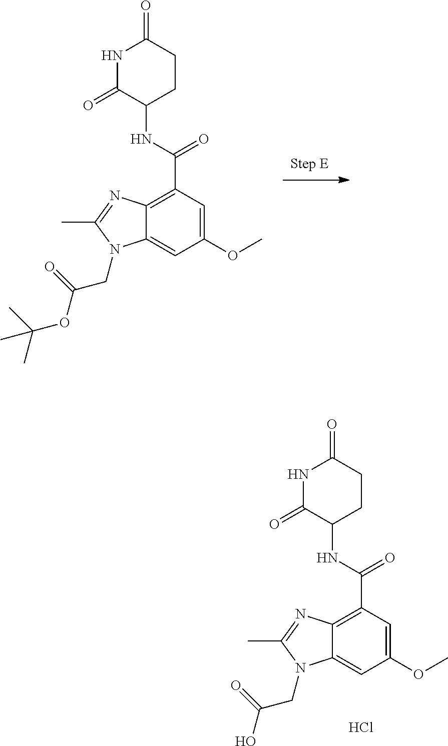 [SMILES: COc1cc(C(=O)NC2CCC(=O)NC2=O)c2nc(C)n(CC(=O)OC(C)(C)C)c2c1 ; COc1cc(C(=O)NC2CCC(=O)NC2=O)c2nc(C)n(CC(=O)O)c2c1 ; Cl]

tert-Butyl 2-{4-[(2,6-dioxopiperidin-3-yl)carbamoyl]-6-methoxy-2-methyl-1H-1,3-benzodiazol-1-yl}acetate (57.0 mg, 0.132 mmol) was dissolved in DCM (0.500 mL) and to the mixture was added TFA (0.500 mL). The reaction was stirred for 18 h at RT. The crude was concentrated in vacuo. To the resulting residue was added 1M HCl aqueous solution (3 mL) and concentrated to dryness under reduced pressure. Crude 2-{4-[(2,6-dioxopiperidin-3-yl)carbamoyl]-6-methoxy-2-methyl-1H-1,3-benzodiazol-1-yl}acetic acid hydrochloride (54.0 mg) was obtained as white solid.

LCMS (ESI+): m/z 375.1 [M+H]⁺

### Step F

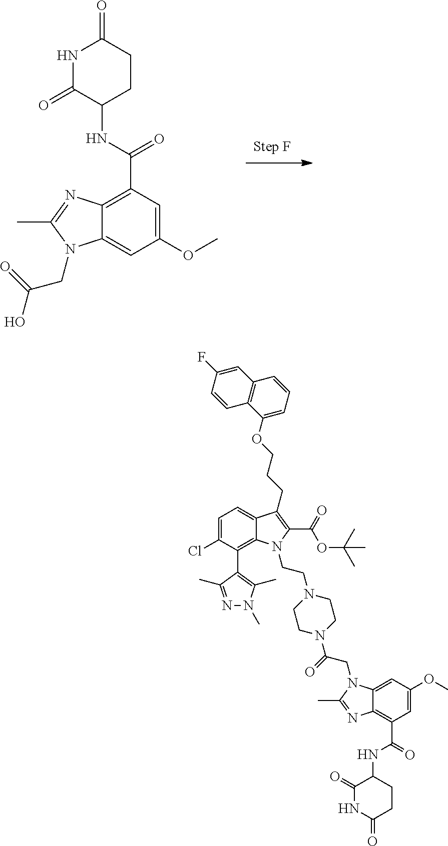

To a solution of crude 2-{4-[(2,6-dioxopiperidin-3-yl)carbamoyl]-6-methoxy-2-methyl-1H-1,3-benzodiazol-1-yl}acetic acid hydrochloride (21.9 mg), tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (30.0 mg, 0.044 mmol) and HATU (33.8 mg, 0.089 mmol) in dry DMF (2.0 mL) was added DIPEA (0.023 mL, 0.133 mmol). The mixture was stirred at room temperature for 30 min. The crude was diluted with DCM and washed with brine. The organic layer was dried over Na₂SO₄, filtered and concentrated in vacuo to get 58 mg crude tert-butyl 6-chloro-1-{2-[4-(2-{4-[(2,6-dioxopiperidin-3-yl)carbamoyl]-6-methoxy-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate as yellow oil which was used in the next step without further purification.

LCMS (ESI+): m/z 1030.3 [M+H]⁺

### Step G

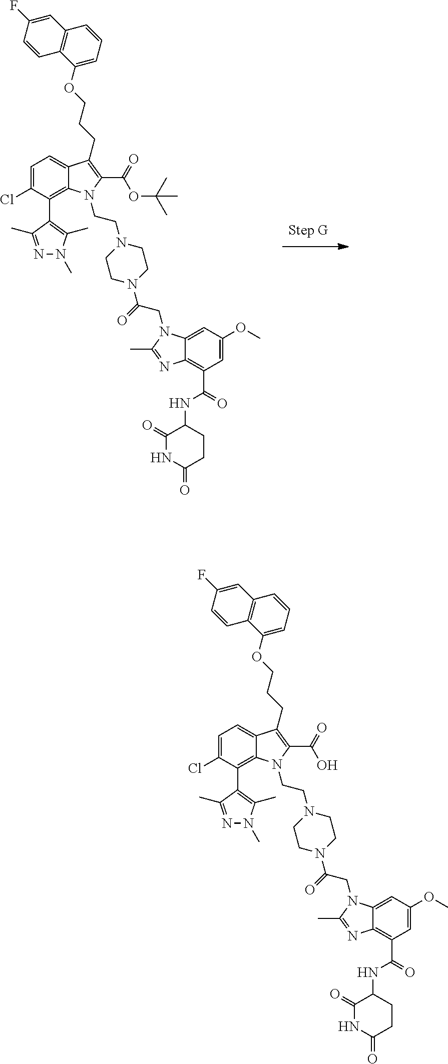

To a solution of tert-butyl 6-chloro-1-{2-[4-(2-{4-[(2,6-dioxopiperidin-3-yl)carbamoyl]-6-methoxy-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (58 mg, crude) in DCM (1.0 mL) was added TFA (1.0 mL). The mixture was stirred for 18 h at RT. The crude was concentrated in vacuo and purified with preparative HPLC (C18, H₂O:MeCN+0.1% FA) to obtain 6-chloro-1-{2-[4-(2-{4-[(2,6-dioxopiperidin-3-yl)carbamoyl]-6-methoxy-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (17.8 mg, 0.018 mmol, 40.9% over two steps) as a white solid.

LCMS (ESI+): m/z 974.4 [M+H]⁺

¹H NMR (500 MHz, DMSO, 353 K) δ 10.56 (s, 1H), 10.14 (d, J=7.2 Hz, 1H), 8.25 (dd, J=9.2, 5.8 Hz, 1H), 7.72 (d, J=8.5 Hz, 1H), 7.59 (dd, J=10.4, 2.6 Hz, 1H), 7.47-7.40 (m, 3H), 7.32 (td, J=8.9, 2.6 Hz, 1H), 7.22 (d, J=8.5 Hz, 1H), 7.19 (d, J=2.5 Hz, 1H), 6.88 (dd, J=5.5, 3.1 Hz, 1H), 5.16 (s, 2H), 4.84 (ddd, J=12.4, 7.3, 5.2 Hz, 1H), 4.34-4.18 (m, 4H), 3.84 (s, 3H), 3.78 (s, 3H), 3.55-3.36 (m, 4H), 3.32-3.27 (m, 2H), 2.80 (ddd, J=17.5, 13.0, 5.5 Hz, 1H), 2.65-2.57 (m, 1H), 2.44 (s, 3H), 2.34-2.06 (m, 10H), 2.03 (s, 3H), 1.91 (s, 3H).

### Example 124. 6-Chloro-1-{2-[4-(2-{4-[(2,6-dioxopiperidin-3-yl)carbamoyl]-6-fluoro-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (278)

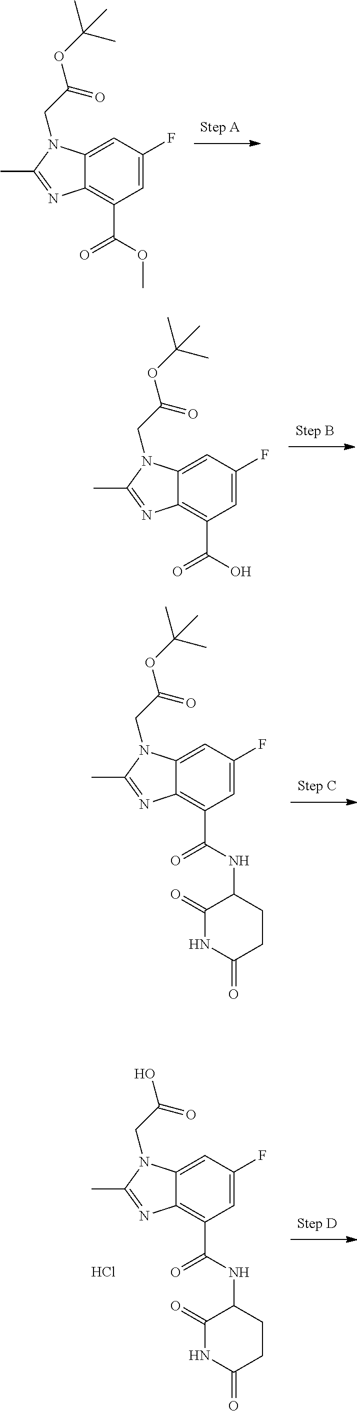

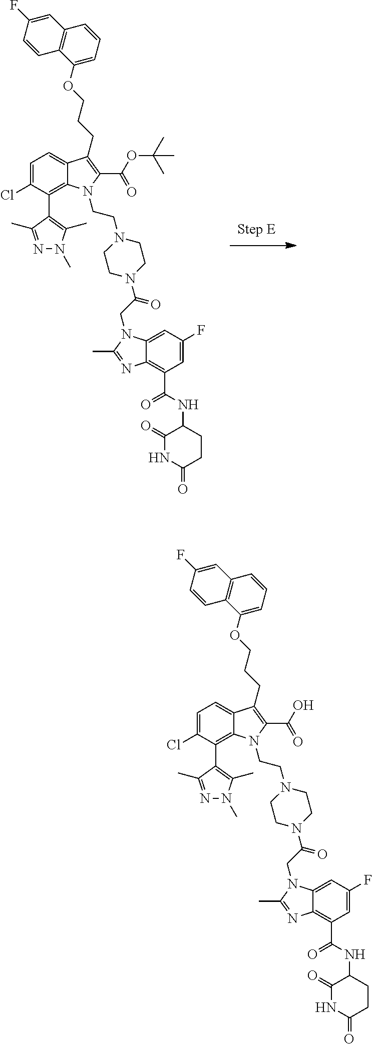 [SMILES: Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)cc(F)cc54)CC3)c12 ; Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cc(F)cc54)CC3)c12]

### Step A

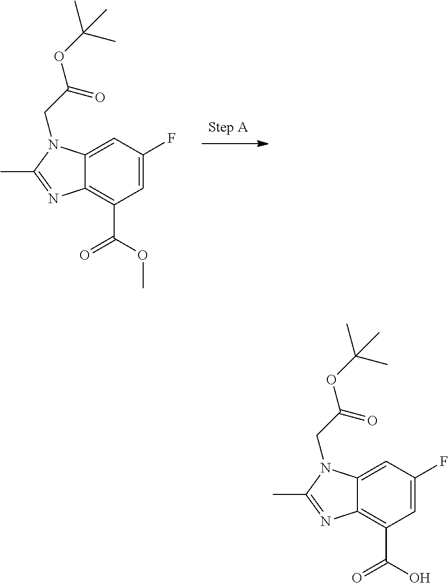 [SMILES: COC(=O)c1cc(F)cc2c1nc(C)n2CC(=O)OC(C)(C)C ; Cc1nc2c(C(=O)O)cc(F)cc2n1CC(=O)OC(C)(C)C]

To a stirred solution of methyl 1-(2-(tert-butoxy)-2-oxoethyl)-6-fluoro-2-methyl-1H-benzo[d]imidazole-4-carboxylate (500 mg, 1.55 mmol) in H₂O (1 mL) and MeCN (9 mL) was added LiBr (4 g, 46.0 mmol) and Et₃N (4.3 mL, 31.06 mmol) at room temperature. The mixture was stirred for 24 h at room temperature. After complete consumption of starting material (monitored by LCMS and TLC), the reaction mixture was concentrated in vacuo at low temperature. The residue was dissolved in water and washed with Et₂O. The aqueous layer was carefully acidified to pH=˜7 at 0° C. and extracted with EtOAc and washed with brine solution. The organic layer was dried over Na₂SO₄, filtered and evaporated under reduced pressure to get crude compound which was purified by triturating with Et₂O to afford 1-[2-(tert-butoxy)-2-oxoethyl]-6-fluoro-2-methyl-1H-1,3-benzodiazole-4-carboxylic acid (372 mg, 1.21 mmol, 78%) as off white solid.

LCMS (ESI+): m/z 309.1 [M+H]⁺

### Step B

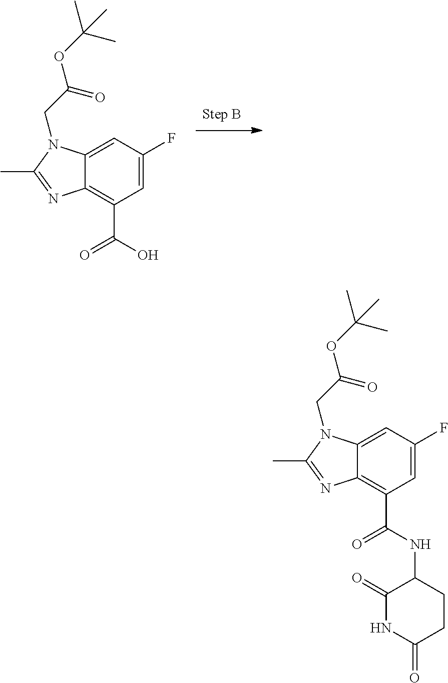 [SMILES: Cc1nc2c(C(=O)O)cc(F)cc2n1CC(=O)OC(C)(C)C ; Cc1nc2c(C(=O)NC3CCC(=O)NC3=O)cc(F)cc2n1CC(=O)OC(C)(C)C]

To a solution of 1-[2-(tert-butoxy)-2-oxoethyl]-6-fluoro-2-methyl-1H-1,3-benzodiazole-4-carboxylic acid (100.0 mg, 0.324 mmol) and 3-aminopiperidine-2,6-dione hydrochloride (64.1 mg, 0.389 mmol) in dry DMF (2.0 mL) was added DIPEA (0.169 mL, 0.973 mmol) and HATU (246.7 mg, 0.649 mmol). The mixture was stirred for 18 h at RT. After this time, the crude was purified by reverse phase chromatography (C18, H₂O:MeCN+0.1% FA) to isolate tert-butyl 2-{4-[(2,6-dioxopiperidin-3-yl)carbamoyl]-6-fluoro-2-methyl-1H-1,3-benzodiazol-1-yl}acetate (104.7 mg, 0.250 mmol, 77%) as off-white solid.

LCMS (ESI+): m/z 419.3 [M+H]⁺

### Step C

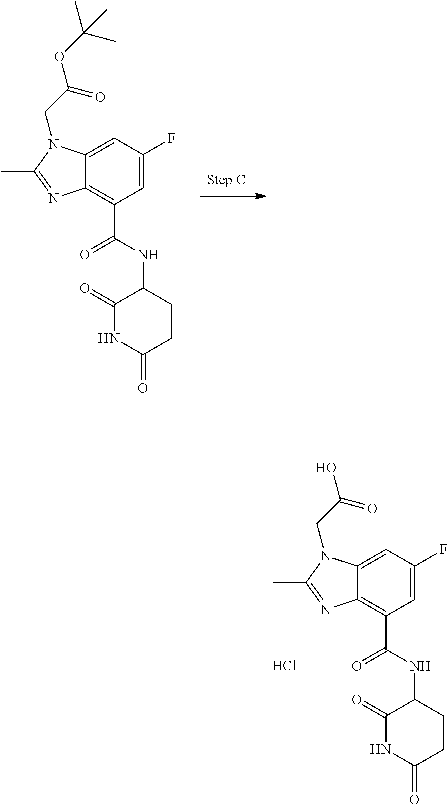 [SMILES: Cc1nc2c(C(=O)NC3CCC(=O)NC3=O)cc(F)cc2n1CC(=O)OC(C)(C)C ; Cc1nc2c(C(=O)NC3CCC(=O)NC3=O)cc(F)cc2n1CC(=O)O ; Cl]

To a solution of tert-butyl 2-{4-[(2,6-dioxopiperidin-3-yl)carbamoyl]-6-fluoro-2-methyl-1H-1,3-benzodiazol-1-yl}acetate (104.7 mg, 0.250 mmol) in DCM (1.0 mL) was added TFA (1.0 mL). The mixture was stirred for 5 h at RT. The crude was concentrated in vacuo and dissolved in H₂O. To the solution was added 36% HCl and it was evaporated. The product, 2-{4-[(2,6-dioxopiperidin-3-yl)carbamoyl]-6-fluoro-2-methyl-1H-1,3-benzodiazol-1-yl}acetic acid hydrochloride (86.0 mg, 0.216 mmol, 86%) was isolated as brownish solid.

LCMS (ESI+): m/z 363.0 [M+H]⁺

### Step D

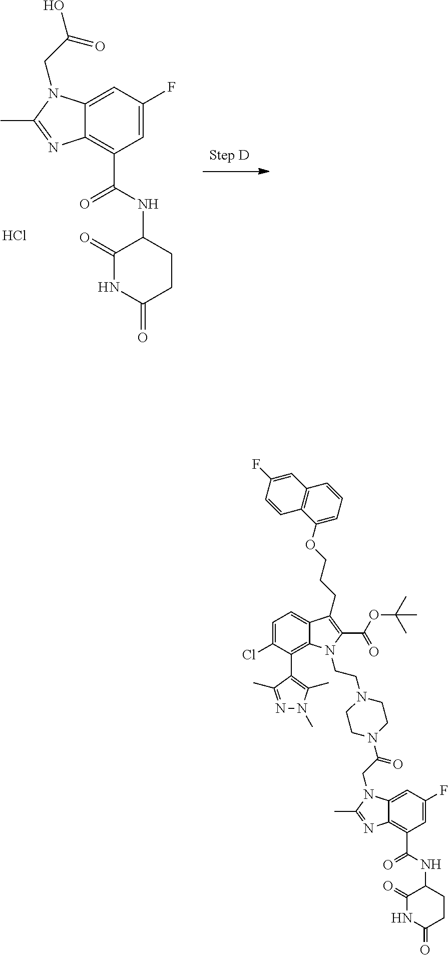 [SMILES: Cc1nc2c(C(=O)NC3CCC(=O)NC3=O)cc(F)cc2n1CC(=O)O ; Cl ; Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)cc(F)cc54)CC3)c12]

To a solution of 2-{4-[(2,6-dioxopiperidin-3-yl)carbamoyl]-6-fluoro-2-methyl-1H-1,3-benzodiazol-1-yl}acetic acid hydrochloride (12.9 mg, 0.032 mmol) and tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (20.0 mg, 0.030 mmol) in dry DMF (5.0 mL) were added HATU (22.6 mg, 0.059 mmol) and DIPEA (0.026 mL, 0.148 mmol). The reaction was stirred at room temperature for 15 min. The crude was diluted with DCM and washed with brine. The organic layer was dried over MgSO₄, filtered and concentrated in vacuo to obtain tert-butyl 6-chloro-1-{2-[4-(2-{4-[(2,6-dioxopiperidin-3-yl)carbamoyl]-6-fluoro-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (65 mg, crude) as an yellow oil, which was used in the next step without further purification.

LCMS (ESI+): m/z 510.4 [M+2H]²⁺

### Step E

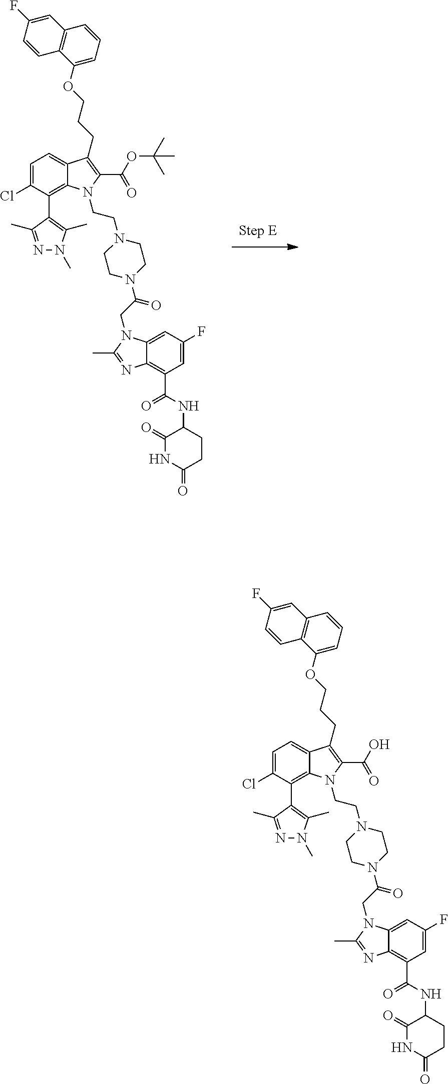 [SMILES: Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)cc(F)cc54)CC3)c12 ; Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cc(F)cc54)CC3)c12]

To a solution of tert-butyl 6-chloro-1-{2-[4-(2-{4-[(2,6-dioxopiperidin-3-yl)carbamoyl]-6-fluoro-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (65 mg, crude) in DCM (1.0 mL) was added TFA (1.0 mL, 13.059 mmol). The reaction mixture was stirred for 18 h at RT. The crude was concentrated in vacuo and purified by preparative HPLC (C18, H₂O:MeCN+0.1% FA) to obtain 6-chloro-1-{2-[4-(2-{4-[(2,6-dioxopiperidin-3-yl)carbamoyl]-6-fluoro-2-methyl-1H-1,3-benzodiazol-1-yl}acetyl)piperazin-1-yl]ethyl}-3-{3-[(6-fIuoronaphthalen-1-yl)oxy]propyl}-7-(1,3,5-trimethyl-1H-pyrazol-4-yl)-1H-indole-2-carboxylic acid (11.2 mg, 0.012 mmol, 40% over 2 steps) as a yellow solid.

LCMS (ESI+): m/z 962.1 [M+H]⁺

¹H NMR (500 MHz, DMSO, 353K) δ 10.59 (s, 1H), 10.12 (d, J=7.2 Hz, 1H), 8.25 (dd, J=9.2, 5.9 Hz, 1H), 7.70 (d, J=8.5 Hz, 1H), 7.60-7.53 (m, 3H), 7.45-7.40 (m, 2H), 7.33 (ddd, J=9.3, 8.7, 2.6 Hz, 1H), 7.21 (d, J=8.5 Hz, 1H), 6.88 (dd, J=5.3, 3.4 Hz, 1H), 5.20 (s, 2H), 4.86 (ddd, J=12.4, 7.2, 5.2 Hz, 1H), 4.34-4.19 (m, 4H), 3.78 (s, 3H), 3.51-3.36 (m, 4H), 3.30-3.26 (m, 2H), 2.81 (ddd, J=17.5, 13.1, 5.5 Hz, 1H), 2.64-2.58 (m, 1H), 2.48 (s, 3H), 2.35-2.08 (m, 10H), 2.03 (s, 3H), 1.91 (s, 3H).

### Example 125. 6-Chloro-1-(2-(4-(2-(4-((2,6-dioxopiperidin-3-yl)carbamoyl)-5-fluoro-2-methyl-1H-benzo[d]imidazol-1-yl)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (279)

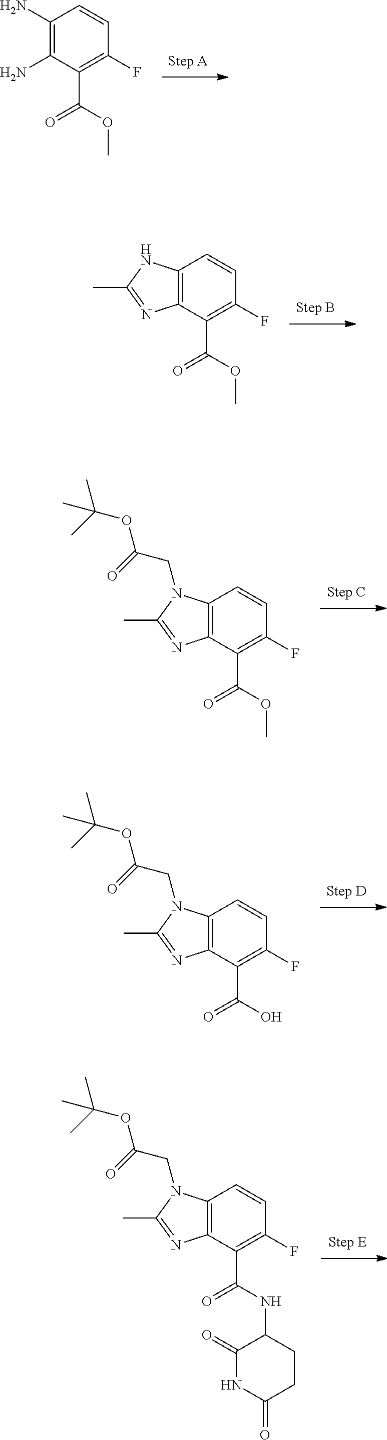 [SMILES: COC(=O)c1c(F)ccc(N)c1N ; COC(=O)c1c(F)ccc2[nH]c(C)nc12 ; COC(=O)c1c(F)ccc2c1nc(C)n2CC(=O)OC(C)(C)C ; Cc1nc2c(C(=O)O)c(F)ccc2n1CC(=O)OC(C)(C)C ; Cc1nc2c(C(=O)NC3CCC(=O)NC3=O)c(F)ccc2n1CC(=O)OC(C)(C)C]

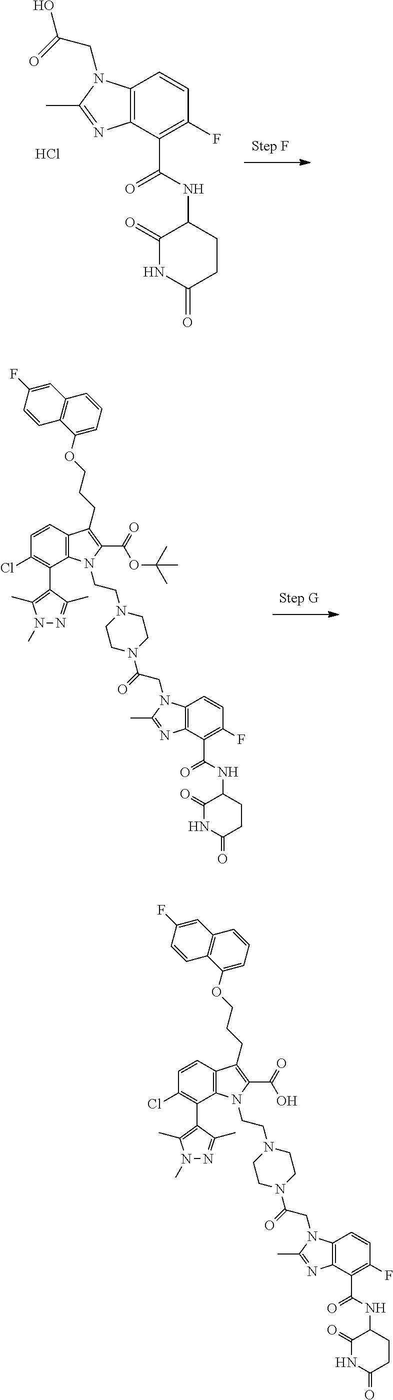 [SMILES: Cc1nc2c(C(=O)NC3CCC(=O)NC3=O)c(F)ccc2n1CC(=O)O ; Cl ; Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)c(F)ccc54)CC3)c12 ; Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)c(F)ccc54)CC3)c12]

### Step A

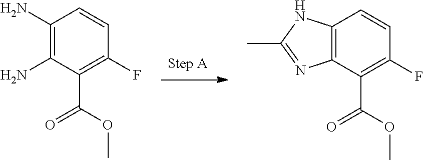

To a solution of methyl 2,3-diamino-6-fluorobenzoate (750 mg, 4.07 mmol) in acetic acid (4.5 mL), was added Et₃N (0.45 mL) at 0° C. and then the reaction mixture was allowed to stir for 12 h at 110° C. After consumption of the starting material (monitored by LCMS and TLC), the volatiles were evaporated under reduced pressure to get the crude compound which was purified by column chromatography (SiO₂, 50% EtOAc in DCM) to get 500 mg (2.40 mmol, 59%) of methyl 5-fluoro-2-methyl-1H-benzo[d]imidazole-4-carboxylate as brown solid powder.

LCMS (ESI+): m/z 208.8[M+H]⁺

### Step B

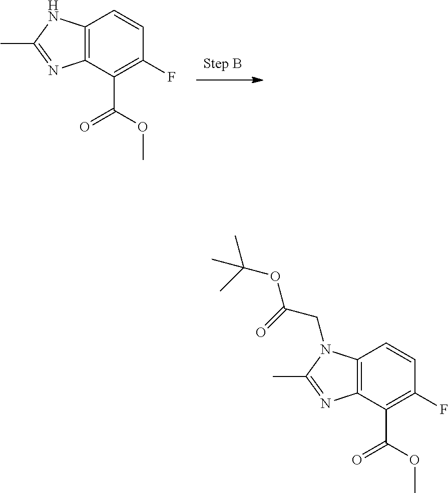 [SMILES: COC(=O)c1c(F)ccc2[nH]c(C)nc12 ; COC(=O)c1c(F)ccc2c1nc(C)n2CC(=O)OC(C)(C)C]

To a solution of methyl 5-fluoro-2-methyl-1H-benzo[d]imidazole-4-carboxylate (500 mg, 2.40 mmol) in THE (15.0 mL) was added sodium hydride (60%) (130 mg, 2.885 mmol) portion wise at 0° C. After stirring the reaction mixture for 30 min was added tert-butyl bromoacetate (0.426 mL, 3.125 mmol) at 0° C. and then the reaction mixture was allowed to slowly reach room temperature and was continued for 1 h under nitrogen. After complete consumption of starting material (monitored by LCMS and TLC), to the reaction was added 5 mL of cold water to quench the unreacted sodium hydride and then the reaction mixture was extracted with EtOAc. The combined organic layer was washed with distilled water and brine, dried over anhydrous Na₂SO₄ and filtered. The filtrate was evaporated under reduced pressure to get the crude compound which was then purified by column chromatography (SiO₂, 40% EtOAc in DCM) to get 500 mg (1.55 mmol, 64%) of methyl 1-(2-(tert-butoxy)-2-oxoethyl)-5-fluoro-2-methyl-1H-benzo[d]imidazole-4-carboxylate as brown solid.

LCMS (ESI+): m/z 323.2 [M+H]⁺

### Step C

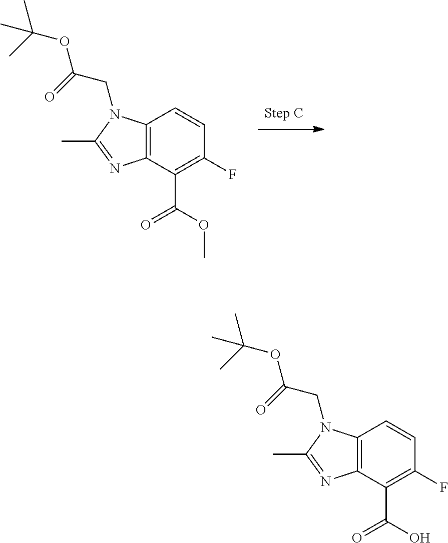 [SMILES: COC(=O)c1c(F)ccc2c1nc(C)n2CC(=O)OC(C)(C)C ; Cc1nc2c(C(=O)O)c(F)ccc2n1CC(=O)OC(C)(C)C]

To a stirred solution of methyl 1-(2-(tert-butoxy)-2-oxoethyl)-5-fluoro-2-methyl-1H-benzo[d]imidazole-4-carboxylate (1 g, 3.1 mmol) in MeCN (40 mL) and H₂O (4 mL), was added LiBr (8.08 g, 93.2 mmol) and Et₃N (8.6 mL, 62.08 mmol) at 0° C. Then, the resulting reaction mixture was warm to room temperature and stirred for 16 h. After complete consumption of starting material (monitored with LCMS), the reaction mixture was evaporated under vacuum at 25° C. The residue was dissolved in water and washed with Et₂O. The aqueous layer was carefully acidified to pH=˜3 at 0° C. and extracted with 10% MeOH in DCM. The organic layer was dried over Na₂SO₄, filtered and evaporated under reduced pressure to get the crude compound which was purified by triturating with Et₂O to afford 0.5 g (1.62 mmol, 52%) of 1-(2-(tert-butoxy)-2-oxoethyl)-5-fluoro-2-methyl-1H-benzo[d]imidazole-4-carboxylic acid as off white solid.

LCMS (ESI+): m/z 309.1 [M+H]⁺

### Step D

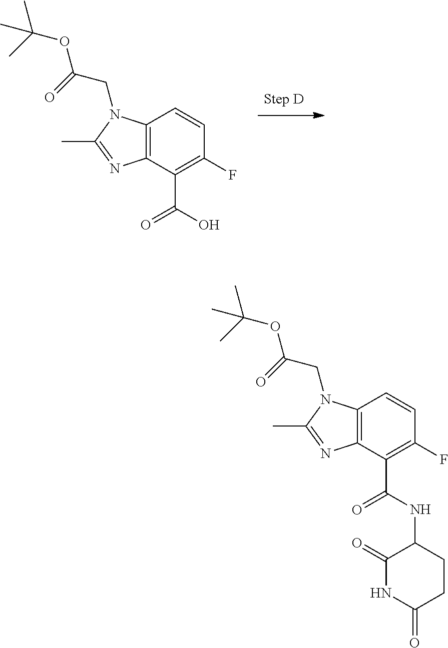 [SMILES: Cc1nc2c(C(=O)O)c(F)ccc2n1CC(=O)OC(C)(C)C ; Cc1nc2c(C(=O)NC3CCC(=O)NC3=O)c(F)ccc2n1CC(=O)OC(C)(C)C]

1-(2-(tert-Butoxy)-2-oxoethyl)-5-fluoro-2-methyl-1H-benzo[d]imidazole-4-carboxylic acid (30.0 mg, 0.097 mmol), 3-aminopiperidine-2,6-dione hydrochloride (19.2 mg, 0.117 mmol) and HATU (74.0 mg, 0.195 mmol) were dissolved in dry DMF (1.2 mL) under argon atmosphere. To it, DIPEA (0.051 mL, 0.292 mmol) was added and the reaction (monitored with LCMS) was stirred for 15 min under argon at room temperature. After complete conversion of the starting material, the reaction was concentrated under reduced pressure. The resulting residue was dissolved in DMSO and purified with reverse phase flash chromatography (C18, H₂O:MeCN+0.1% FA) to afford tert-butyl 2-{4-[(2,6-dioxopiperidin-3-yl)carbamoyl]-5-fluoro-2-methyl-1H-1,3-benzodiazol-1-yl}acetate (22.9 mg, 0.055 mmol, 56%) as off white solid.

LCMS (ESI+): m/z 419.3 [M+H]⁺

### Step E

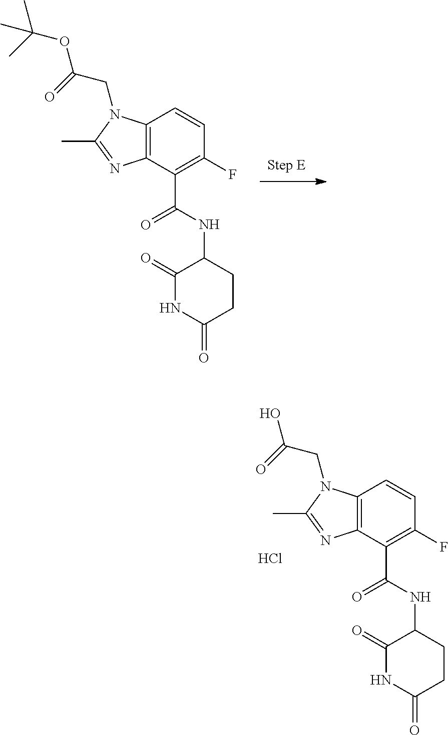 [SMILES: Cc1nc2c(C(=O)NC3CCC(=O)NC3=O)c(F)ccc2n1CC(=O)OC(C)(C)C ; Cc1nc2c(C(=O)NC3CCC(=O)NC3=O)c(F)ccc2n1CC(=O)O ; Cl]

tert-Butyl 2-{4-[(2,6-dioxopiperidin-3-yl)carbamoyl]-5-fluoro-2-methyl-1H-1,3-benzodiazol-1-yl}acetate (22.9 mg, 0.055 mmol) was dissolved in dry DCM (0.500 mL) under argon atmosphere and TFA (0.500 mL, 6.529 mmol) was added. Reaction (monitored with LCMS) was stirred for 4 h at room temperature. After complete conversion of the starting material, the volatiles were evaporated and to the resulting residue an aqueous solution of 1 M HCl (1 mL) was added, followed by concentrating the mixture do dryness under reduced pressure. The addition and evaporation of 1M HCl_{aq} was repeated twice. Resulting product was freeze-dried to give 2-{4-[(2,6-dioxopiperidin-3-yl)carbamoyl]-5-fluoro-2-methyl-1H-1,3-benzodiazol-1-yl}acetic acid hydrochloride (21.8 mg, 0.055 mmol, 99.7%) as pale yellow solid.

LCMS (ESI+): m/z 363.0 [M+H]⁺

### Step F

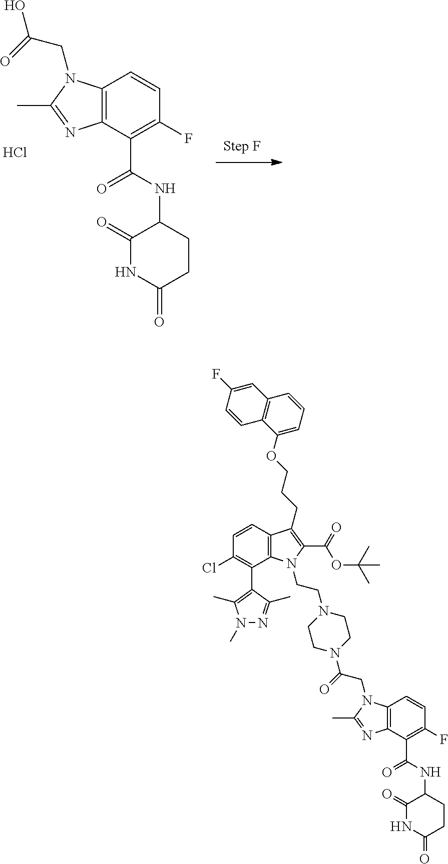 [SMILES: Cc1nc2c(C(=O)NC3CCC(=O)NC3=O)c(F)ccc2n1CC(=O)O ; Cl ; Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)c(F)ccc54)CC3)c12]

tert-Butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (25.0 mg, 0.037 mmol) and 2-{4-[(2,6-dioxopiperidin-3-yl)carbamoyl]-5-fluoro-2-methyl-1H-1,3-benzodiazol-1-yl}acetic acid hydrochloride (22.2 mg, 0.056 mmol) were dissolved in dry DMF (1.2 mL) under argon atmosphere and DIPEA (0.019 mL, 0.111 mmol) followed by HATU (14.8 mg, 0.039 mmol) were added. Reaction (monitored with LCMS) was stirred for 15 min under argon atmosphere at room temperature. After complete conversion of the starting material, the solution was diluted with DCM and washed with brine and water. The organic layer was dried over anhydrous MgSO₄, filtered and dried under reduced pressure to give crude tert-butyl 6-chloro-1-{2-[4-(2-{4-[(2,6-dioxopiperidin-3-yl)carbamoyl]-5-fluoro-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (45.5 mg) as yellow oil. Crude product was directly used in the subsequent step.

LCMS (ESI+): m/z 1018.2 [M+H]

### Step G

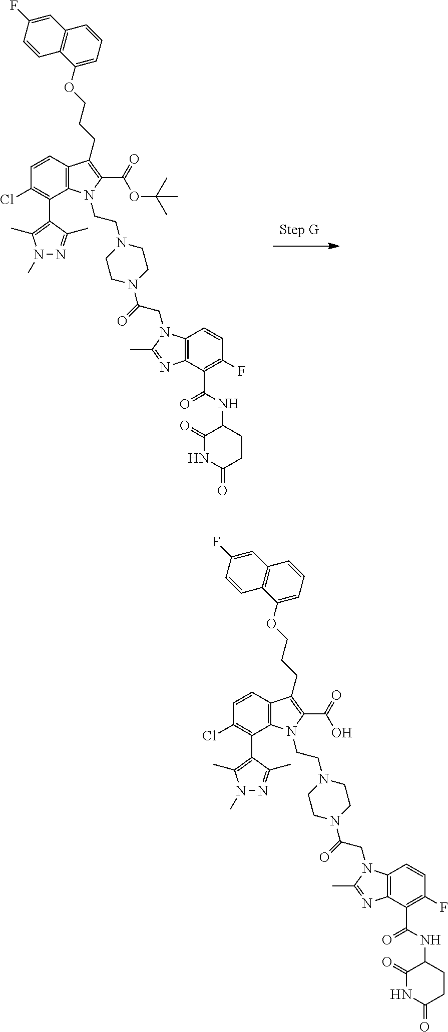 [SMILES: Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)c(F)ccc54)CC3)c12 ; Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)c(F)ccc54)CC3)c12]

tert-Butyl 6-chloro-1-{2-[4-(2-{4-[(2,6-dioxopiperidin-3-yl)carbamoyl]-5-fluoro-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (45.5 mg, crude) was dissolved in dry DCM (0.400 mL) under argon atmosphere and TFA (0.400 mL, 5.224 mmol) was added. Reaction (monitored with LCMS) was stirred under argon for 16 h at room temperature. After complete conversion of the starting material, the solution was concentrated under reduced pressure and dissolved in DMSO. Crude product was purified with preparative HPLC (C18, H₂O:MeCN+0.1% FA) to give 6-chloro-1-(2-(4-(2-(4-((2,6-dioxopiperidin-3-yl)carbamoyl)-5-fluoro-2-methyl-1H-benzo[d]imidazol-1-yl)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (9.8 mg, 0.010 mmol, 27% over 2 steps) as white solid.

LCMS (ESI+): m/z 962.2 [M+H]⁺

¹H NMR (500 MHz, DMSO, 353 K) δ 10.54 (s, 1H), 9.83 (d, J=7.2 Hz, 1H), 8.25 (dd, J=9.3, 5.9 Hz, 1H), 7.71 (d, J=8.6 Hz, 1H), 7.59 (dd, J=10.4, 2.6 Hz, 1H), 7.54 (dd, J=8.8, 3.9 Hz, 1H), 7.45-7.40 (m, 2H), 7.33 (td, J=8.9, 2.6 Hz, 1H), 7.22 (d, J=8.5 Hz, 1H), 7.06 (dd, J=11.8, 8.8 Hz, 1H), 6.88 (dd, J=5.5, 3.1 Hz, 1H), 5.19 (s, 2H), 4.80 (ddd, J=12.1, 7.3, 5.2 Hz, 1H), 4.35-4.17 (m, 4H), 3.78 (s, 3H), 3.43 (bs, 4H), 3.29 (dd, J=8.4, 6.5 Hz, 2H), 2.79 (ddd, J=17.9, 12.7, 5.5 Hz, 1H), 2.66-2.57 (m, 1H), 2.46 (s, 3H), 2.31-2.05 (m, 10H), 2.03 (s, 3H), 1.91 (s, 3H).

### Example 126. 6-Chloro-1-[3-(2-{4-[(2,6-dioxopiperidin-3-yl)carbamoyl]-2-methyl-1H-1,3-benzodiazol-1-yl}ethoxy)propyl]-3-{3-[(6-fluoronaphthalen-1-yl)oxy]propyl}-7-(1,3,5-trimethyl-1H-pyrazol-4-yl)-1H-indole-2-carboxylic acid (280)

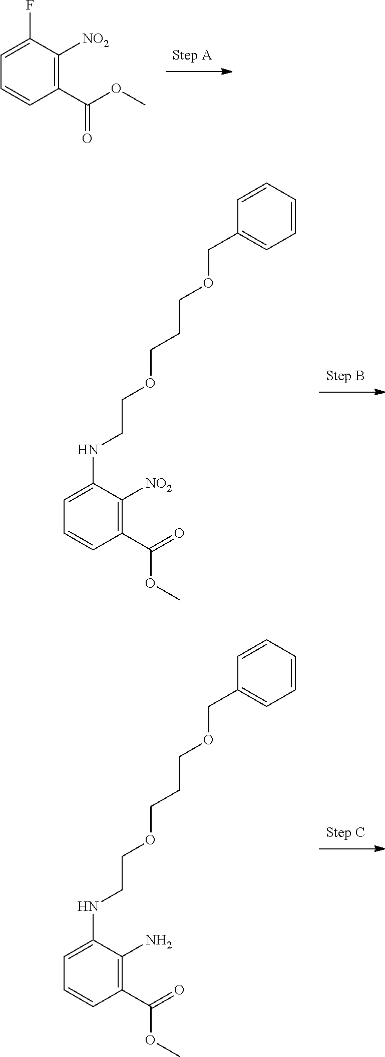 [SMILES: COC(=O)c1cccc(F)c1[N+](=O)[O-] ; COC(=O)c1cccc(NCCOCCCOCc2ccccc2)c1[N+](=O)[O-] ; COC(=O)c1cccc(NCCOCCCOCc2ccccc2)c1N]

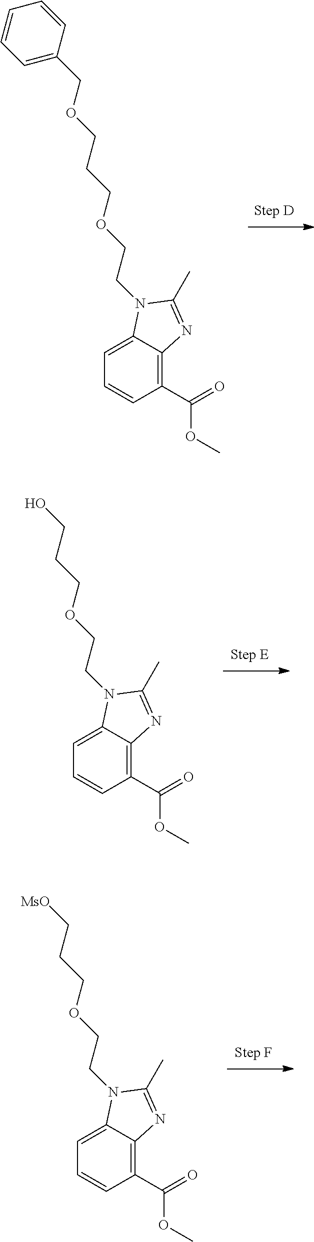 [SMILES: COC(=O)c1cccc2c1nc(C)n2CCOCCCOCc1ccccc1 ; COC(=O)c1cccc2c1nc(C)n2CCOCCCO ; COC(=O)c1cccc2c1nc(C)n2CCOCCCOS(C)(=O)=O]

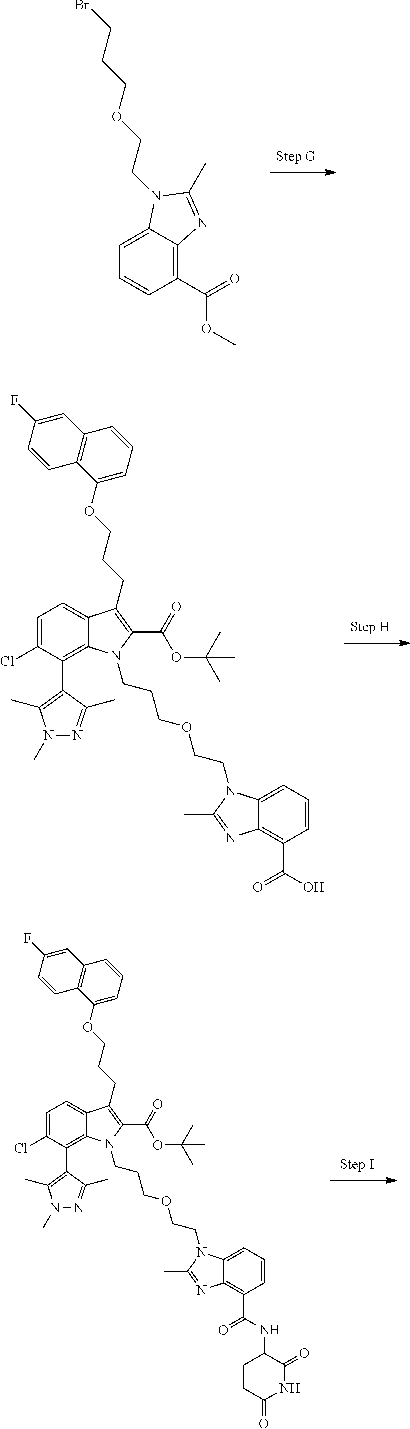 [SMILES: COC(=O)c1cccc2c1nc(C)n2CCOCCCBr ; Cc1nn(C)c(C)c1-c1c(Cl)ccc2c(CCCOc3cccc4cc(F)ccc34)c(C(=O)OC(C)(C)C)n(CCCOCCn3c(C)nc4c(C(=O)O)cccc43)c12 ; Cc1nn(C)c(C)c1-c1c(Cl)ccc2c(CCCOc3cccc4cc(F)ccc34)c(C(=O)OC(C)(C)C)n(CCCOCCn3c(C)nc4c(C(=O)NC5CCC(=O)NC5=O)cccc43)c12]

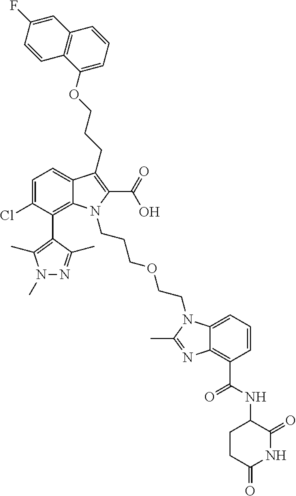 [SMILES: Cc1nn(C)c(C)c1-c1c(Cl)ccc2c(CCCOc3cccc4cc(F)ccc34)c(C(=O)O)n(CCCOCCn3c(C)nc4c(C(=O)NC5CCC(=O)NC5=O)cccc43)c12]

### Step A

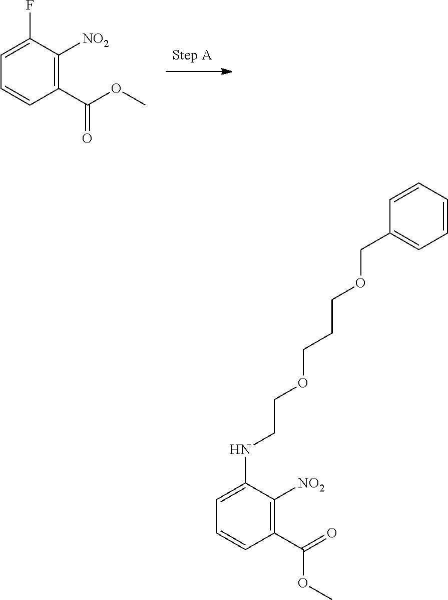 [SMILES: COC(=O)c1cccc(F)c1[N+](=O)[O-] ; COC(=O)c1cccc(NCCOCCCOCc2ccccc2)c1[N+](=O)[O-]]

To a well stirred solution of methyl 3-fluoro-2-nitrobenzoate (1.6 g, 8.04 mmol) in DMSO (30 mL) was added 2-(3-(benzyloxy)propoxy)ethan-1-amine (2.0 g, 9.56 mmol) and DIPEA (4 mL, 24.12 mmol) in sealed tube at room temperature under nitrogen. The reaction mixture was allowed to stir at 70° C. for 3 h. After complete consumption of the starting material, (monitored by LCMS), the reaction mixture was diluted with EtOAc and washed successively with water and brine. The organic layer was dried over Na₂SO₄ and evaporated under reduced pressure to get the crude compound, which was then purified by column chromatography (SiO₂, 20% EtOAc in hexane) to obtain methyl 3-((2-(3-(benzyloxy)propoxy)ethyl)amino)-2-nitrobenzoate (2.5 g, 6.44 mmol, 80%) as yellow solid.

LCMS (ESI+): m/z 389.2 [M+H]⁺

### Step B

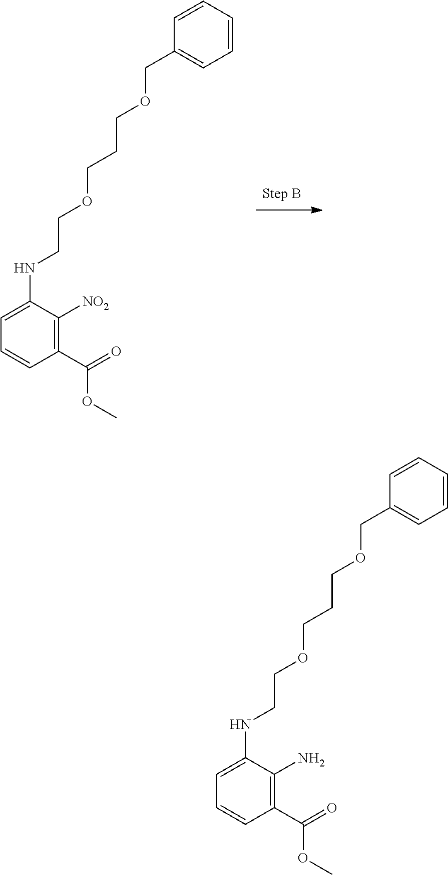 [SMILES: COC(=O)c1cccc(NCCOCCCOCc2ccccc2)c1[N+](=O)[O-] ; COC(=O)c1cccc(NCCOCCCOCc2ccccc2)c1N]

To a stirred solution of methyl 3-((2-(3-(benzyloxy)propoxy)ethyl)amino)-2-nitrobenzoate (2.5 g, 6.44 mmol) in MeOH (40 mL) was added Fe powder (3.6 g, 64.43 mmol) and a solution of NH₄Cl (3.4 g, 64.43 mmol) in water (10 mL) and the reaction was allowed to stir at 70° C. for 16 h. After complete consumption of the starting material (monitored by LCMS), the mixture was cooled to room temperature and filtered through Celite® pad, which was then washed with MeOH. The volatiles were evaporated under reduced pressure and the crude material was diluted with EtOAc. Organic layer was washed successively with water and brine, dried over Na₂SO₄ and evaporated under reduced pressure. Crude compound was purified with column chromatography (SiO₂, 30% EtOAc in hexane) to obtain methyl 2-amino-3-((2-(3-(benzyloxy)propoxy)ethyl)amino)benzoate (2.0 g, 5.58 mmol, 86.6%) as brown solid.

LCMS (ESI+): m/z 359.0 [M+H]⁺

### Step C

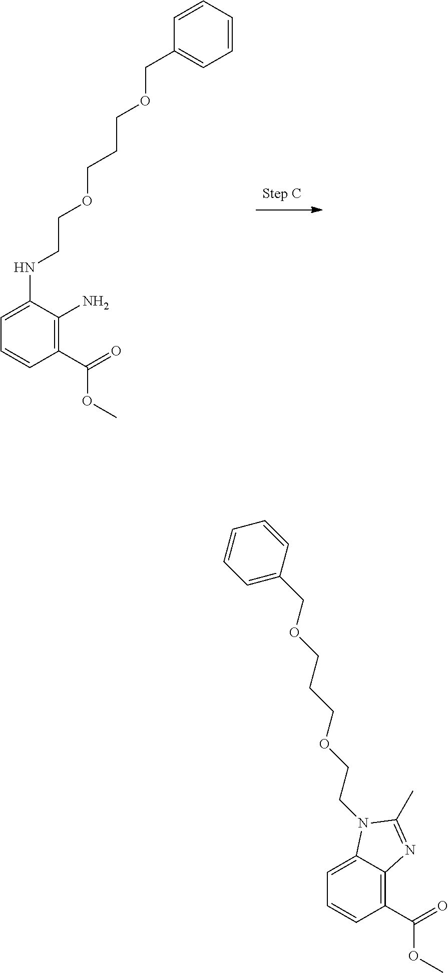 [SMILES: COC(=O)c1cccc(NCCOCCCOCc2ccccc2)c1N ; COC(=O)c1cccc2c1nc(C)n2CCOCCCOCc1ccccc1]

To a solution of methyl 2-amino-3-((2-(3-(benzyloxy)propoxy)ethyl)amino)benzoate (2.0 g, 5.58 mmol) in hexafluoro isopropanol (10 mL) was added trimethyl orthoacetate (7.5 mL, 44.70 mmol) at room temperature under nitrogen atmosphere. The resulting solution was stirred for 18 h at RT. After complete consumption of the starting material (monitored by LCMS) the reaction was evaporated under reduced pressure and the residue was dissolved in DCM. Crude product was purified with column chromatography (SiO₂, 2% MeOH in DCM) to obtain methyl 1-(2-(3-(benzyloxy)propoxy)ethyl)-2-methyl-1H-benzo[d]imidazole-4-carboxylate (1.9 g, 4.97 mmol, 89%) of as gummy liquid.

LCMS (ESI+): m/z 382.7 [M+H]⁺

### Step D

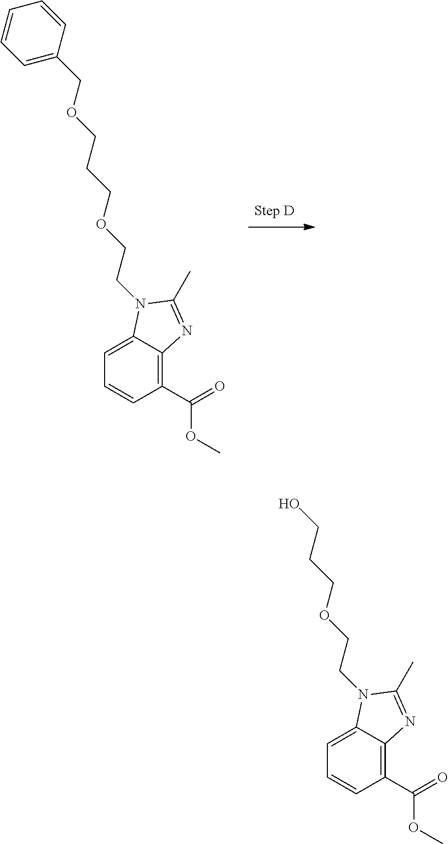 [SMILES: COC(=O)c1cccc2c1nc(C)n2CCOCCCOCc1ccccc1 ; COC(=O)c1cccc2c1nc(C)n2CCOCCCO]

Methyl 1-(2-(3-(benzyloxy)propoxy)ethyl)-2-methyl-1H-benzo[d]imidazole-4-carboxylate (1.9 g, 4.97 mmol) was placed in a 250 mL parr shaker flask and immersed in 50 mL MeOH, followed by Pd/C (20% wt). The reaction mixture was stirred at room temperature under hydrogen atmosphere at 65 psi pressure. After complete consumption of the starting material (monitored by LCMS) the reaction mixture was filtered through Celite® pad and the filtrate was concentrated under reduced pressure to get 750 mg crude of methyl 1-(2-(3-hydroxypropoxy)ethyl)-2-methyl-1H-benzo[d]imidazole-4-carboxylate which was then used for the next step without further purification.

LCMS (ESI+): m/z 293.5 [M+H]⁺

### Step E

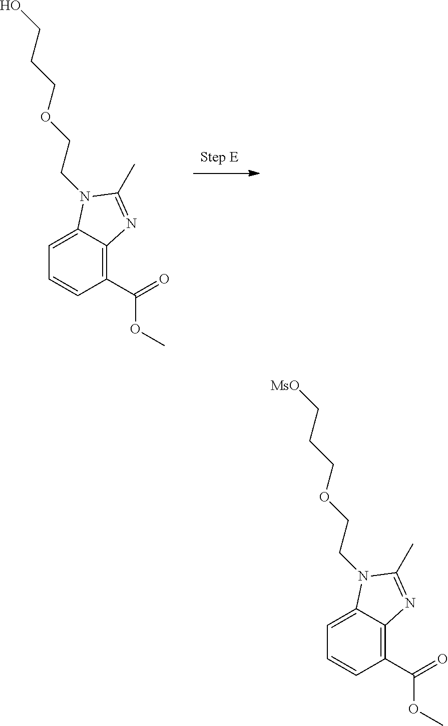 [SMILES: COC(=O)c1cccc2c1nc(C)n2CCOCCCO ; COC(=O)c1cccc2c1nc(C)n2CCOCCCOS(C)(=O)=O]

To a well stirred solution of methyl 1-(2-(3-hydroxypropoxy)ethyl)-2-methyl-1H-benzo[d]imidazole-4-carboxylate (750 mg, crude) in DCM (20 mL) was added MsCl (0.4 mL, 5.17 mmol) and Et₃N (1.1 mL, 7.70 mmol) at 0° C. under nitrogen. The reaction mixture was then allowed to stir at ambient temperature for 2 h. After complete consumption of the starting material (monitored by LCMS) the excess MsCl was quenched with saturated NaHCO₃ solution and extracted with DCM (2 times). Organic layer was washed with water and brine, dried over Na₂SO₄ and evaporated under reduced pressure to get 500 mg crude of methyl 2-methyl-1-(2-(3-((methylsulfonyl)oxy)propoxy)ethyl)-1H-benzo[d]imidazole-4-carboxylate, which was used for the next step without further purification.

LCMS (ESI+): m/z 370.6 [M+H]⁺

### Step F

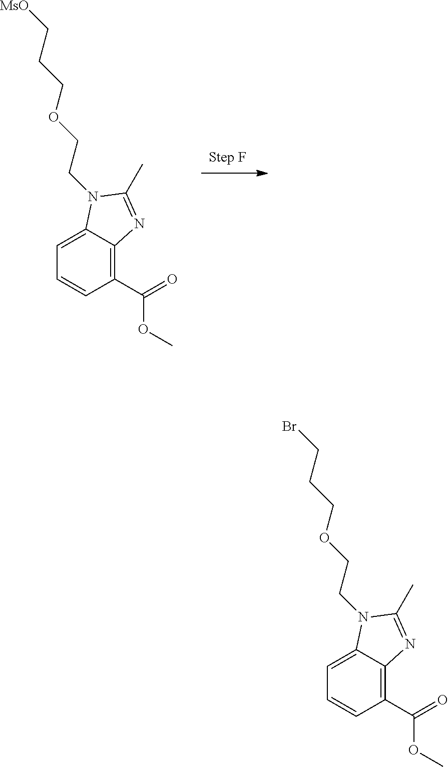 [SMILES: COC(=O)c1cccc2c1nc(C)n2CCOCCCOS(C)(=O)=O ; COC(=O)c1cccc2c1nc(C)n2CCOCCCBr]

To a stirred solution of methyl 2-methyl-1-(2-(3-((methylsulfonyl)oxy)propoxy)ethyl)-1H-benzo[d]imidazole-4-carboxylate (500 mg, crude) in DMF (5 mL) was added LiBr (586 mg, 6.76 mmol) at room temperature under nitrogen. The reaction mixture was allowed to stir at 80° C. for 2 h. After complete consumption of the starting material (monitored by LCMS) the reaction mixture was then diluted with EtOAc, washed successively with cold water (3 times) and brine solution. The organic layer was dried over Na₂SO₄ and evaporated under reduced pressure to get the crude material which was then purified by column chromatography (SiO₂, 4% MeOH in DCM) to obtain methyl 1-(2-(3-bromopropoxy)ethyl)-2-methyl-1H-benzo[d]imidazole-4-carboxylate (200 mg, 0.563 mmol, 11.3% over three steps) as brown solid.

LCMS (ESI+): m/z 355.3 & 357.3 [M+H]⁺

### Step G

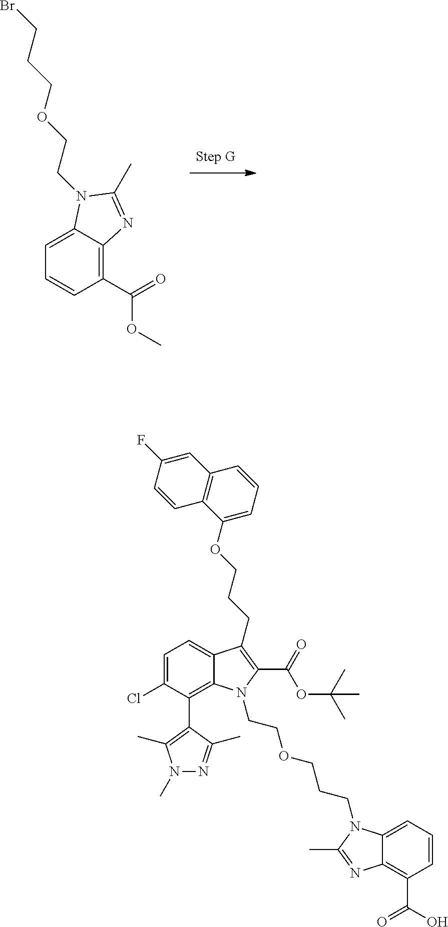 [SMILES: COC(=O)c1cccc2c1nc(C)n2CCOCCCBr ; Cc1nn(C)c(C)c1-c1c(Cl)ccc2c(CCCOc3cccc4cc(F)ccc34)c(C(=O)OC(C)(C)C)n(CCOCCCn3c(C)nc4c(C(=O)O)cccc43)c12]

To a well stirred solution of tert-butyl 6-chloro-3-(3-((6-fluoronaphthalen-1-yl)oxy)propyl)-7-(1,3,5-trimethyl-1H-pyrazol-4-yl)-1H-indole-2-carboxylate (200 mg, 0.36 mmol) in DMF (5 mL) was added methyl 1-(2-(3-bromopropoxy)ethyl)-2-methyl-1H-benzo[d]imidazole-4-carboxylate (164 mg, 0.46 mmol) followed by Cs₂CO₃ (357 mg, 1.07 mmol) and the mixture was allowed to stir at 90° C. for 16 h under nitrogen. After complete consumption of the starting material (monitored by LCMS), the reaction mixture was acidified with 1 (N) HCl and extracted with EtOAc to get the crude material which was purified by preparative HPLC (C18, H₂O:MeCN+0.1% FA) to afford 1-[2-(3-{2-[(tert-butoxy)carbonyl]-6-chloro-3-{3-[(6-fluoronaphthalen-1-yl)oxy]propyl}-7-(1,3,5-trimethyl-1H-pyrazol-4-yl)-1H-indol-1-yl}propoxy)ethyl]-2-methyl-1H-1,3-benzodiazole-4-carboxylic acid (105 mg, 0.127 mmol, 36%) as white solid.

LCMS (ESI+): m/z 822.7 [M+H]⁺

### Step H

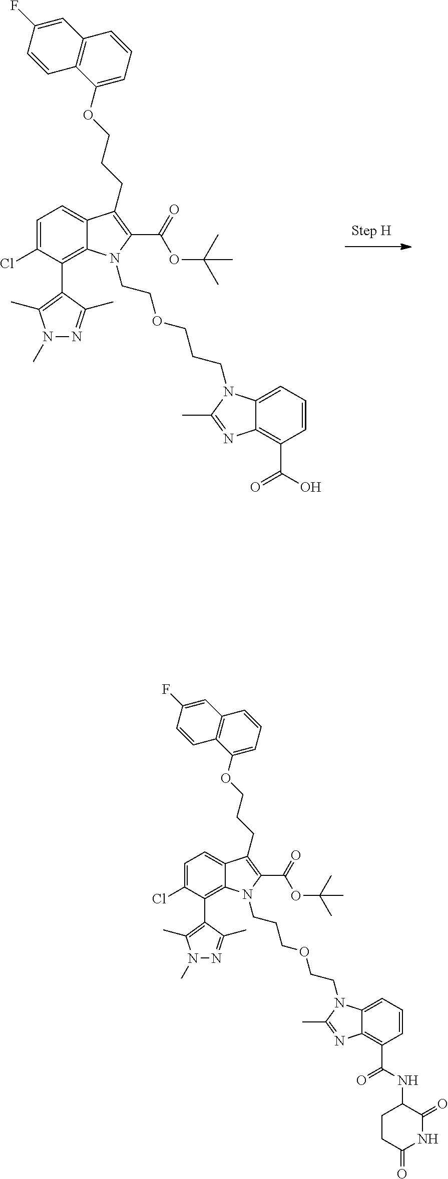 [SMILES: Cc1nn(C)c(C)c1-c1c(Cl)ccc2c(CCCOc3cccc4cc(F)ccc34)c(C(=O)OC(C)(C)C)n(CCOCCCn3c(C)nc4c(C(=O)O)cccc43)c12 ; Cc1nn(C)c(C)c1-c1c(Cl)ccc2c(CCCOc3cccc4cc(F)ccc34)c(C(=O)OC(C)(C)C)n(CCCOCCn3c(C)nc4c(C(=O)NC5CCC(=O)NC5=O)cccc43)c12]

To the mixture of 1-[2-(3-{2-[(tert-butoxy)carbonyl]-6-chloro-3-{3-[(6-fluoronaphthalen-1-yl)oxy]propyl}-7-(1,3,5-trimethyl-1H-pyrazol-4-yl)-1H-indol-1-yl}propoxy)ethyl]-2-methyl-1H-1,3-benzodiazole-4-carboxylic acid (50 mg, 0.061 mmol), 3-aminopiperidine-2,6-dione hydrochloride (12.0 mg, 0.073 mmol) and HATU (46.2 mg, 0.122 mmol) in DMF (2.0 mL) was added DIPEA (0.032 mL, 0.182 mmol). The reaction was stirred at room temperature for 30 min. The crude was diluted with DCM, washed with brine, dried over Na₂SO₄, filtered and concentrated in vacuo to get tert-butyl 6-chloro-1-[3-(2-{4-[(2,6-dioxopiperidin-3-yl)carbamoyl]-2-methyl-1H-1,3-benzodiazol-1-yl}ethoxy)propyl]-3-{3-[(6-fluoronaphthalen-1-yl)oxy]propyl}-7-(1,3,5-trimethyl-1H-pyrazol-4-yl)-1H-indole-2-carboxylate (84.0 mg, crude), which was used to next step without further purification.

LCMS (ESI+): m/z 932.5 [M+H]⁺

### Step I

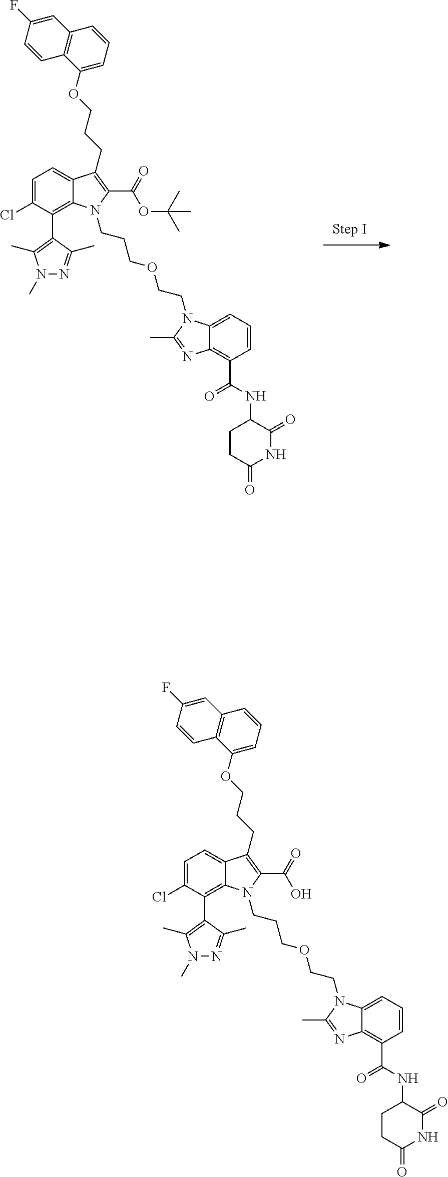 [SMILES: Cc1nn(C)c(C)c1-c1c(Cl)ccc2c(CCCOc3cccc4cc(F)ccc34)c(C(=O)OC(C)(C)C)n(CCCOCCn3c(C)nc4c(C(=O)NC5CCC(=O)NC5=O)cccc43)c12 ; Cc1nn(C)c(C)c1-c1c(Cl)ccc2c(CCCOc3cccc4cc(F)ccc34)c(C(=O)O)n(CCCOCCn3c(C)nc4c(C(=O)NC5CCC(=O)NC5=O)cccc43)c12]

To a solution of tert-butyl 6-chloro-1-[3-(2-{4-[(2,6-dioxopiperidin-3-yl)carbamoyl]-2-methyl-1H-1,3-benzodiazol-1-yl}ethoxy)propyl]-3-{3-[(6-fluoronaphthalen-1-yl)oxy]propyl}-7-(1,3,5-trimethyl-1H-pyrazol-4-yl)-1H-indole-2-carboxylate (84 mg, crude) in DCM (1.0 mL) was added TFA (1.0 mL). The mixture was stirred for 18 h at RT. The crude was concentrated in vacuo and purified with preparative HPLC (C18, H₂O:MeCN+0.1% FA) to give 6-chloro-1-[3-(2-{4-[(2,6-dioxopiperidin-3-yl)carbamoyl]-2-methyl-1H-1,3-benzodiazol-1-yl}ethoxy)propyl]-3-{3-[(6-fluoronaphthalen-1-yl)oxy]propyl}-7-(1,3,5-trimethyl-1H-pyrazol-4-yl)-1H-indole-2-carboxylic acid (35.5 mg, 0.041 mmol, 67% over two steps) as a white solid.

LCMS (ESI+): m/z 876.3 [M+H]⁺

¹H NMR (500 MHz, DMSO) δ 13.34 (s, 1H), 10.90 (s, 1H), 10.23 (d, J=7.2 Hz, 1H), 8.23 (dd, J=9.3, 5.9 Hz, 1H), 7.83 (d, J=7.6 Hz, 1H), 7.74 (d, J=8.5 Hz, 1H), 7.70 (d, J=8.0 Hz, 1H), 7.65 (dd, J=10.4, 2.7 Hz, 1H), 7.47-7.39 (m, 2H), 7.36 (td, J=8.9, 2.7 Hz, 1H), 7.29 (t, J=7.6 Hz, 1H), 7.22 (d, J=8.5 Hz, 1H), 6.88 (dd, J=5.7, 3.1 Hz, 1H), 4.87 (dt, J=12.8, 6.3 Hz, 1H), 4.41-4.33 (m, 2H), 4.21 (t, J=6.1 Hz, 2H), 4.14-4.04 (m, 1H), 3.98 (d, J=7.7 Hz, 1H), 3.56 (d, J=3.9 Hz, 3H), 3.55-3.45 (m, 2H), 2.86-2.75 (m, 2H), 2.70-2.62 (m, 1H), 2.59 (s, 3H), 2.57-2.52 (m, 1H), 2.25-2.15 (m, 3H), 2.16-2.05 (m, 1H), 1.85-1.75 (m, 6H), 1.38-1.25 (m, 2H).

2 protons in aliphatic area overlap with water.

### Example 127. 6-Chloro-1-[3-(2-{4-[(2,6-dioxopiperidin-3-yl)carbamoyl]-2-methyl-1H-1,3-benzodiazol-1-yl}acetamido)propyl]-3-{3-[(6-fluoronaphthalen-1-yl)oxy]propyl}-7-(1,3,5-trimethyl-1H-pyrazol-4-yl)-1H-indole-2-carboxylic acid (281)

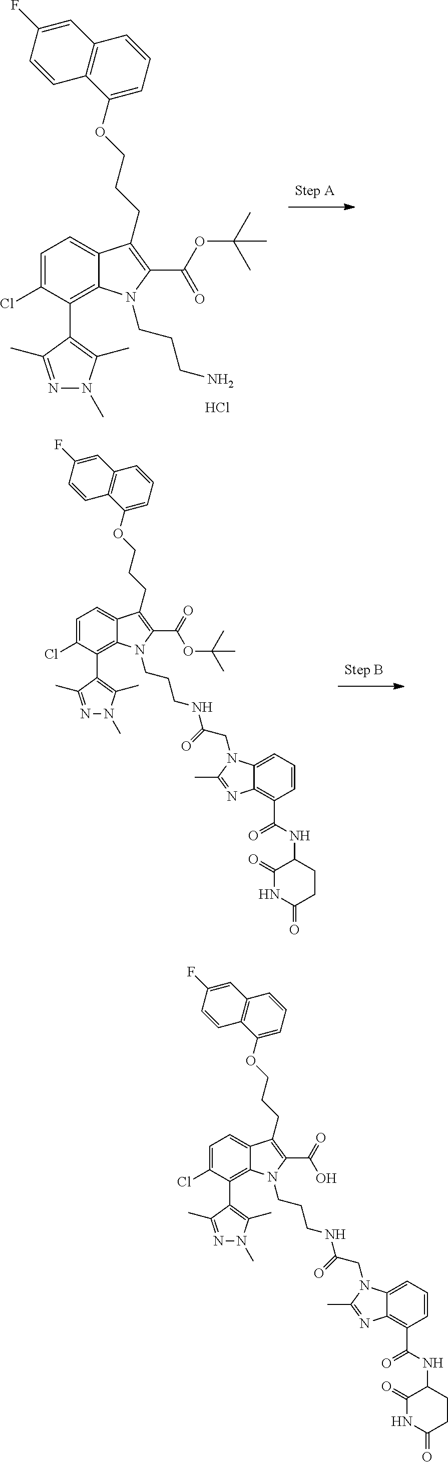 [SMILES: Cc1nn(C)c(C)c1-c1c(Cl)ccc2c(CCCOc3cccc4cc(F)ccc34)c(C(=O)OC(C)(C)C)n(CCCN)c12 ; Cl ; Cc1nn(C)c(C)c1-c1c(Cl)ccc2c(CCCOc3cccc4cc(F)ccc34)c(C(=O)OC(C)(C)C)n(CCCNC(=O)Cn3c(C)nc4c(C(=O)NC5CCC(=O)NC5=O)cccc43)c12 ; Cc1nn(C)c(C)c1-c1c(Cl)ccc2c(CCCOc3cccc4cc(F)ccc34)c(C(=O)O)n(CCCNC(=O)Cn3c(C)nc4c(C(=O)NC5CCC(=O)NC5=O)cccc43)c12]

### Step A

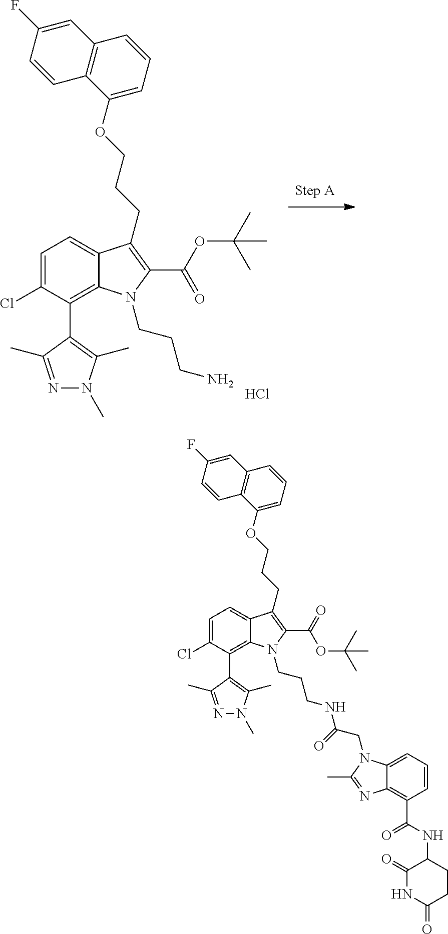 [SMILES: Cc1nn(C)c(C)c1-c1c(Cl)ccc2c(CCCOc3cccc4cc(F)ccc34)c(C(=O)OC(C)(C)C)n(CCCN)c12 ; Cl ; Cc1nn(C)c(C)c1-c1c(Cl)ccc2c(CCCOc3cccc4cc(F)ccc34)c(C(=O)OC(C)(C)C)n(CCCNC(=O)Cn3c(C)nc4c(C(=O)NC5CCC(=O)NC5=O)cccc43)c12]

To a solution of tert-butyl 1-(3-aminopropyl)-6-chloro-3-{3-[(6-fluoronaphthalen-1-yl)oxy]propyl}-7-(1,3,5-trimethyl-1H-pyrazol-4-yl)-1H-indole-2-carboxylate hydrochloride (15.0 mg, crude), 2-{4-[(2,6-dioxopiperidin-3-yl)carbamoyl]-2-methyl-1H-1,3-benzodiazol-1-yl}acetic acid hydrochloride (10.5 mg, 0.027 mmol) and HATU (17.4 mg, 0.046 mmol) in dry DMF (2.0 mL) was added DIPEA (0.012 mL, 0.069 mmol). The mixture was stirred at room temperature for 30 min. The crude was diluted with DCM and washed with brine. The organic layer was dried over MgSO₄, filtered and concentrated in vacuo to get 35 mg crude tert-butyl 6-chloro-1-[3-(2-{4-[(2,6-dioxopiperidin-3-yl)carbamoyl]-2-methyl-1H-1,3-benzodiazol-1-yl}acetamido)propyl]-3-{3-[(6-fluoronaphthalen-1-yl)oxy]propyl}-7-(1,3,5-trimethyl-1H-pyrazol-4-yl)-1H-indole-2-carboxylate as orange oil which was used in the next step without further purification.

LCMS (ESI+): m/z 945.1 [M+H]⁺

### Step B

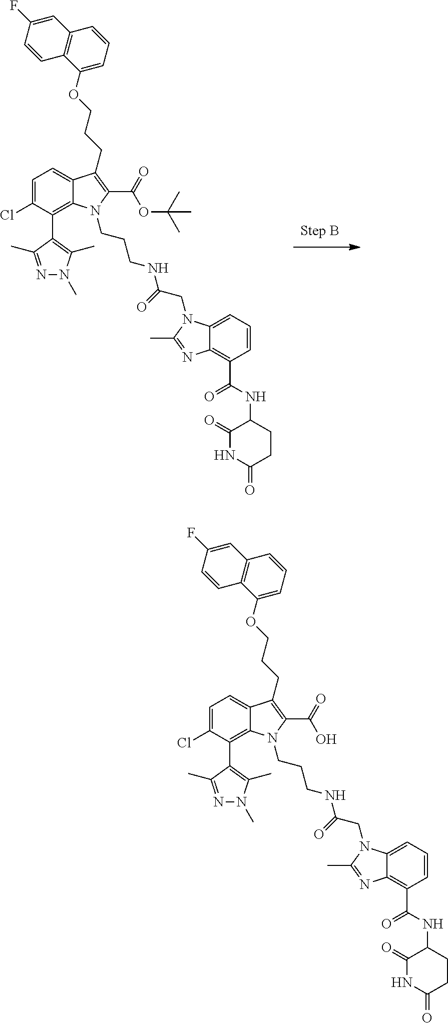 [SMILES: Cc1nn(C)c(C)c1-c1c(Cl)ccc2c(CCCOc3cccc4cc(F)ccc34)c(C(=O)OC(C)(C)C)n(CCCNC(=O)Cn3c(C)nc4c(C(=O)NC5CCC(=O)NC5=O)cccc43)c12 ; Cc1nn(C)c(C)c1-c1c(Cl)ccc2c(CCCOc3cccc4cc(F)ccc34)c(C(=O)O)n(CCCNC(=O)Cn3c(C)nc4c(C(=O)NC5CCC(=O)NC5=O)cccc43)c12]

To a solution of tert-butyl 6-chloro-1-[3-(2-{4-[(2,6-dioxopiperidin-3-yl)carbamoyl]-2-methyl-1H-1,3-benzodiazol-1-yl}acetamido)propyl]-3-{3-[(6-fluoronaphthalen-1-yl)oxy]propyl}-7-(1,3,5-trimethyl-1H-pyrazol-4-yl)-1H-indole-2-carboxylate (35 mg, crude) in DCM (1.0 mL) was added TFA (1.0 mL, 13.059 mmol). The mixture was stirred for 18 h at RT. The crude was concentrated in vacuo and purified by preparative HPLC (C18, H₂O:MeCN+0.1% FA) to obtain 6-chloro-1-[3-(2-{4-[(2,6-dioxopiperidin-3-yl)carbamoyl]-2-methyl-1H-1,3-benzodiazol-1-yl}acetamido)propyl]-3-{3-[(6-fluoronaphthalen-1-yl)oxy]propyl}-7-(1,3,5-trimethyl-1H-pyrazol-4-yl)-1H-indole-2-carboxylic acid (5.0 mg, 0.006 mmol, 40% over four steps—ligand 330) as a white solid.

LCMS (ESI+): m/z 889.2 [M+H]⁺

¹H NMR (500 MHz, DMSO, 353 K) δ 12.79 (s, 1H), 10.57 (s, 1H), 10.16 (d, J=7.2 Hz, 1H), 8.26 (dd, J=9.2, 5.9 Hz, 1H), 7.97-7.92 (m, 1H), 7.86 (dd, J=7.6, 1.1 Hz, 1H), 7.71 (d, J=8.5 Hz, 1H), 7.62-7.56 (m, 2H), 7.46-7.38 (m, 2H), 7.34 (td, J=8.9, 2.7 Hz, 1H), 7.29 (t, J=7.8 Hz, 1H), 7.21 (d, J=8.6 Hz, 1H), 6.87 (dd, J=6.5, 2.1 Hz, 1H), 4.91-4.80 (m, 3H), 4.24 (t, J=6.3 Hz, 2H), 4.18 (ddd, J=14.2, 9.8, 5.6 Hz, 2H), 4.05 (ddd, J=14.6, 9.7, 5.6 Hz, 1H), 3.75 (s, 3H), 3.33-3.26 (m, 2H), 2.86-2.71 (m, 2H), 2.68-2.57 (m, 2H), 2.56 (s, 3H), 2.31 (dtd, J=12.8, 5.5, 2.9 Hz, 1H), 2.27-2.18 (m, 2H), 2.12 (qd, J=12.8, 4.7 Hz, 1H), 1.96 (d, J=1.1 Hz, 3H), 1.88 (s, 3H), 1.46-1.39 (m, 1H), 1.39-1.31 (m, 1H).

### Example 128. 6-Chloro-1-[2-(3-{4-[(2,6-dioxopiperidin-3-yl)carbamoyl]-2-methyl-1H-1,3-benzodiazol-1-yl}propoxy)ethyl]-3-{3-[(6-fluoronaphthalen-1-yl)oxy]propyl}-7-(1,3,5-trimethyl-1H-pyrazol-4-yl)-1H-indole-2-carboxylic acid (282)

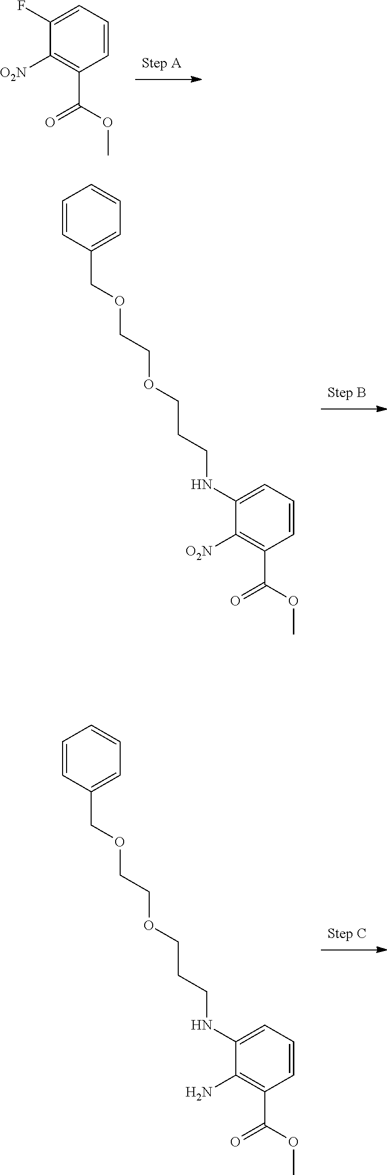

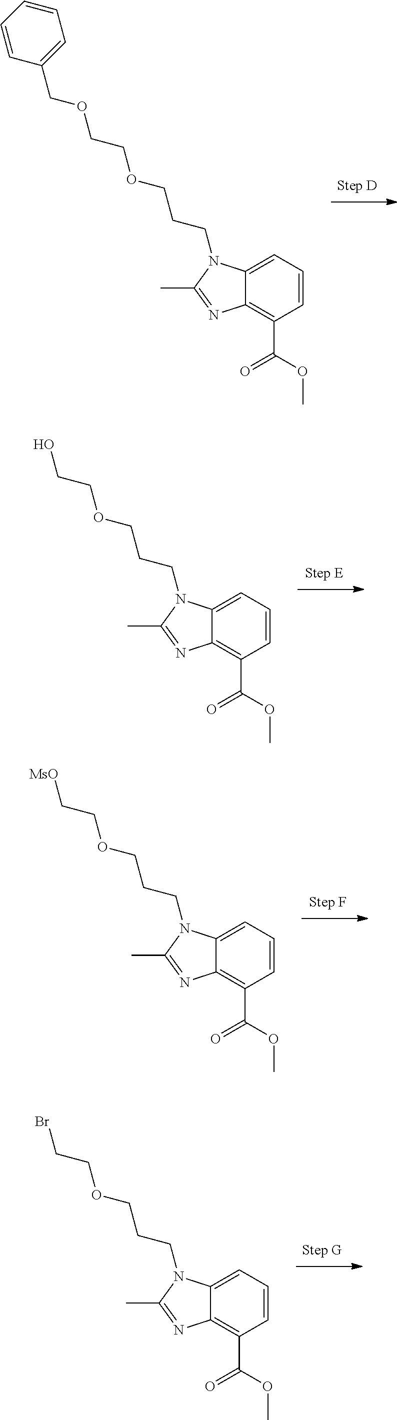 [SMILES: COC(=O)c1cccc2c1nc(C)n2CCCOCCOCc1ccccc1 ; COC(=O)c1cccc2c1nc(C)n2CCCOCCO ; COC(=O)c1cccc2c1nc(C)n2CCCOCCOS(C)(=O)=O ; COC(=O)c1cccc2c1nc(C)n2CCCOCCBr]

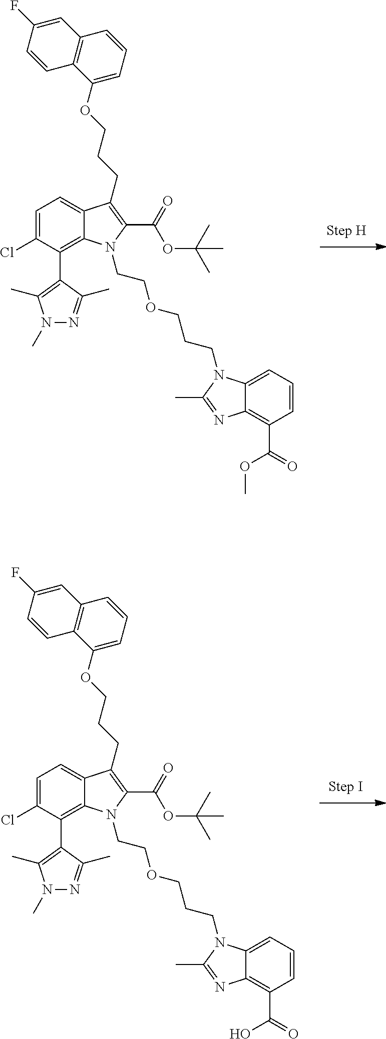 [SMILES: COC(=O)c1cccc2c1nc(C)n2CCCOCCn1c(C(=O)OC(C)(C)C)c(CCCOc2cccc3cc(F)ccc23)c2ccc(Cl)c(-c3c(C)nn(C)c3C)c21 ; Cc1nn(C)c(C)c1-c1c(Cl)ccc2c(CCCOc3cccc4cc(F)ccc34)c(C(=O)OC(C)(C)C)n(CCOCCCn3c(C)nc4c(C(=O)O)cccc43)c12]

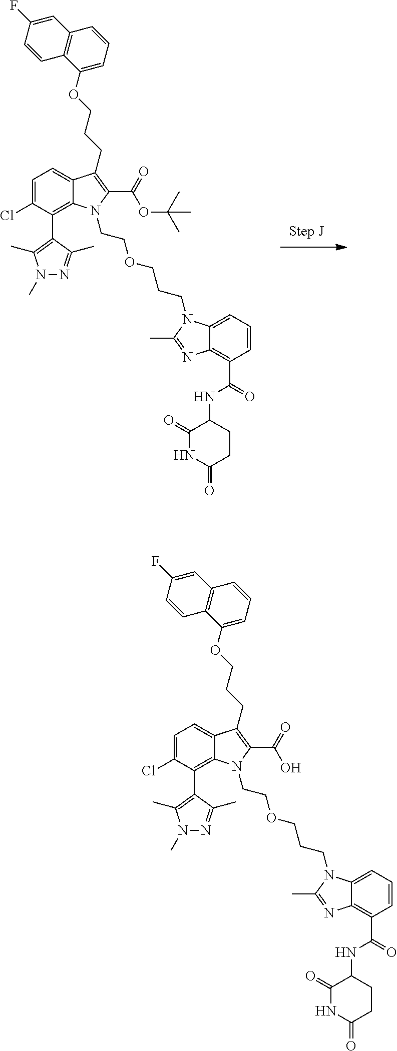 [SMILES: Cc1nn(C)c(C)c1-c1c(Cl)ccc2c(CCCOc3cccc4cc(F)ccc34)c(C(=O)OC(C)(C)C)n(CCOCCCn3c(C)nc4c(C(=O)NC5CCC(=O)NC5=O)cccc43)c12 ; Cc1nn(C)c(C)c1-c1c(Cl)ccc2c(CCCOc3cccc4cc(F)ccc34)c(C(=O)O)n(CCOCCCn3c(C)nc4c(C(=O)NC5CCC(=O)NC5=O)cccc43)c12]

### Step A

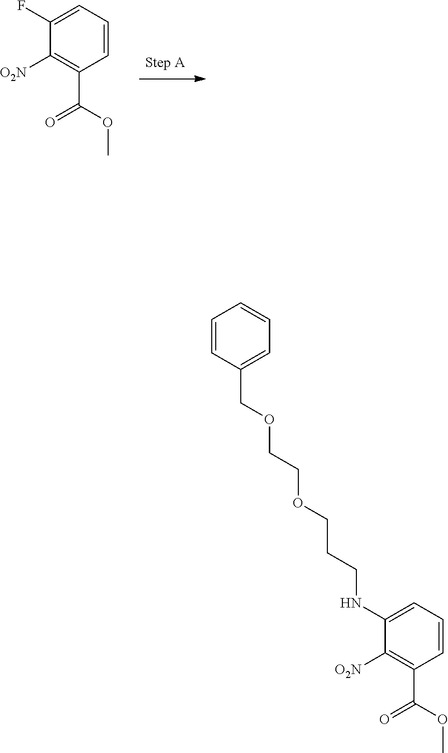 [SMILES: COC(=O)c1cccc(F)c1[N+](=O)[O-] ; COC(=O)c1cccc(NCCCOCCOCc2ccccc2)c1[N+](=O)[O-]]

To a solution of methyl 3-fluoro-2-nitrobenzoate (600.0 mg, 3.013 mmol) in DMSO (8.0 mL) was added 3-(2-(benzyloxy)ethoxy)propan-1-amine (662 mg, 3.164 mmol) and DIPEA (1.504 mL, 9.039 mmol) in sealed tube at room temperature under nitrogen. The reaction mixture was allowed to stir at 70° C. for 3 h. After complete consumption of the starting material (monitored by LCMS) the reaction mixture was diluted with EtOAc and washed successively with water and brine. The organic layer was dried over Na₂SO₄ and evaporated under reduced pressure to get the crude compound, which was then purified by column chromatography (SiO₂, 20% EtOAc in hexane) to obtain methyl 3-((3-(2-(benzyloxy)ethoxy)propyl)amino)-2-nitrobenzoate (1.1 g, 2.83 mmol, 94%) as yellow solid.

LCMS (ESI+): m/z 389.0 [M+H]⁺

### Step B

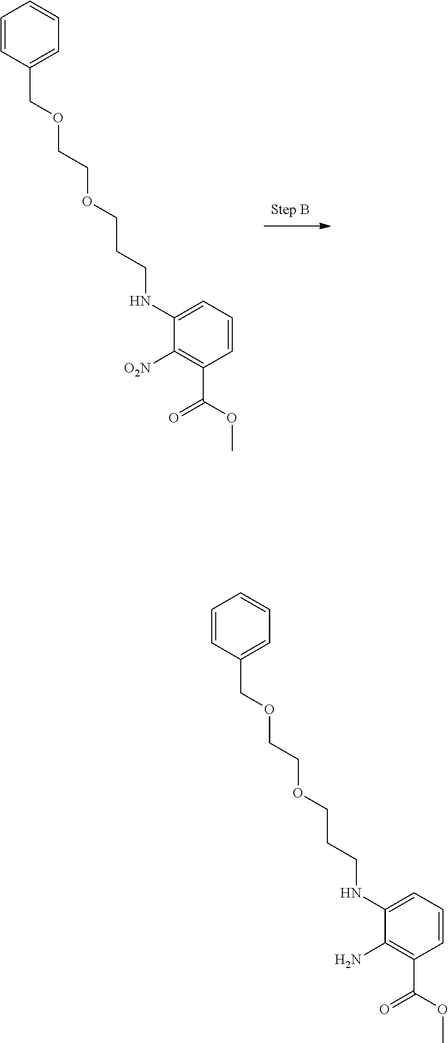 [SMILES: COC(=O)c1cccc(NCCCOCCOCc2ccccc2)c1[N+](=O)[O-] ; COC(=O)c1cccc(NCCCOCCOCc2ccccc2)c1N]

A solution of methyl 3-((3-(2-(benzyloxy)ethoxy)propyl)amino)-2-nitrobenzoate (750 mg, 1.93 mmol) in MeOH (50.0 mL) was deoxygenated using nitrogen for 15 minutes in a parr shaker vessel. After that 10% Pd/C (700.0 mg) was added and the reaction vessel was backfilled with hydrogen. The reaction mixture was then allowed to stir for 2 h at room temperature at 50 psi pressure. After complete consumption of the starting material, (monitored by TLC), the reaction mixture was filtered through Celite® pad and the filtrate was concentrated to get 650 mg of crude methyl 2-amino-3-((3-(2-(benzyloxy)ethoxy)propyl)amino)benzoate which was used for the next step without further purification.

LCMS (ESI+): m/z 359.0 [M+H]⁺

### Step C

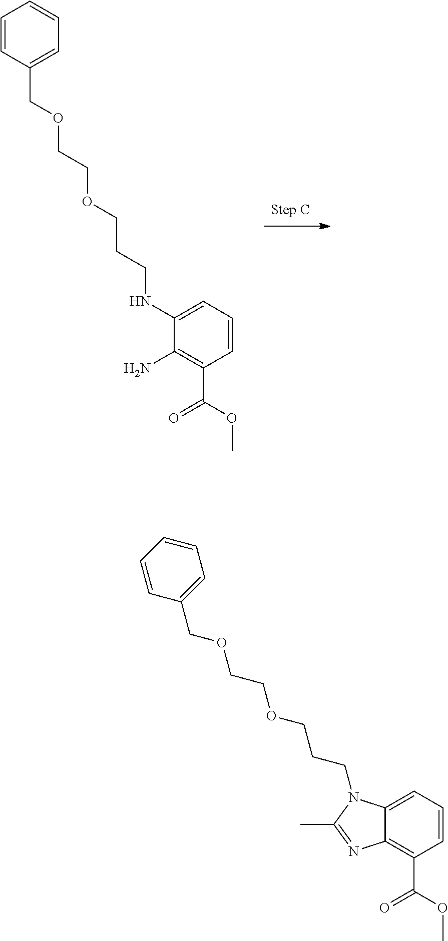 [SMILES: COC(=O)c1cccc(NCCCOCCOCc2ccccc2)c1N ; COC(=O)c1cccc2c1nc(C)n2CCCOCCOCc1ccccc1]

To a solution of methyl 2-amino-3-((3-(2-(benzyloxy)ethoxy)propyl)amino)benzoate (650 mg, crude) in HFIP (5.0 mL) was added trimethyl orthoacetate (1.818 mL, 14.525 mmol) at room temperature under nitrogen atmosphere. The resulting solution was stirred for 18 h at RT. After complete consumption of the starting material (monitored by LCMS) the reaction was evaporated under reduced pressure to get 800.0 mg of crude methyl 1-(3-(2-(benzyloxy)ethoxy)propyl)-2-methyl-1H-benzo[d]imidazole-4-carboxylate as gummy liquid which was used for the next step without further purification.

LCMS (ESI+): m/z 382.8 [M+H]⁺

### Step D

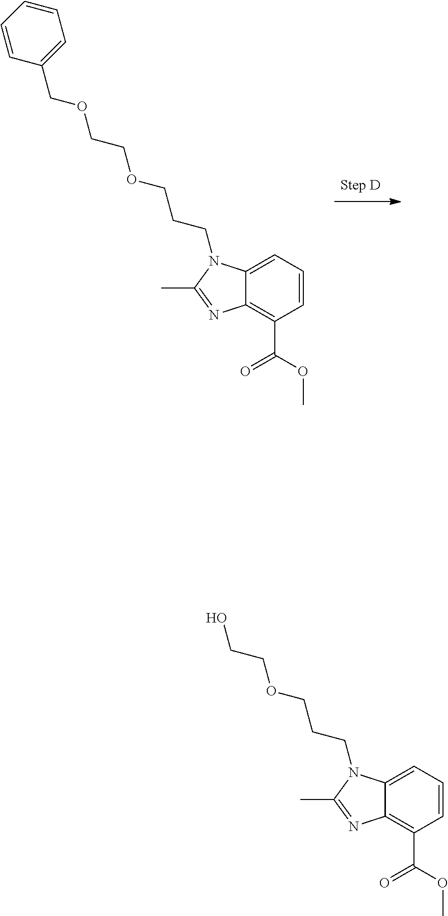 [SMILES: COC(=O)c1cccc2c1nc(C)n2CCCOCCOCc1ccccc1 ; COC(=O)c1cccc2c1nc(C)n2CCCOCCO]

Methyl 1-(3-(2-(benzyloxy)ethoxy)propyl)-2-methyl-1H-benzo[d]imidazole-4-carboxylate (800 mg, crude) was dissolved in MeOH (50 mL) and then deoxygenated using nitrogen for 15 minutes in a 250 mL parr shaker vessel. After that 10% Pd/C (700.0 mg) was added to the reaction vessel and the reaction vessel was backfilled with hydrogen. The reaction mixture was then allowed to stir for 16 h at room temperature under 50 psi pressure. After complete consumption of the starting material the reaction mixture was evaporated under reduced pressure to get 500.0 mg crude of methyl 1-(3-(2-hydroxyethoxy)propyl)-2-methyl-1H-benzo[d]imidazole-4-carboxylate which was then used for the next step without further purification.

### Step E

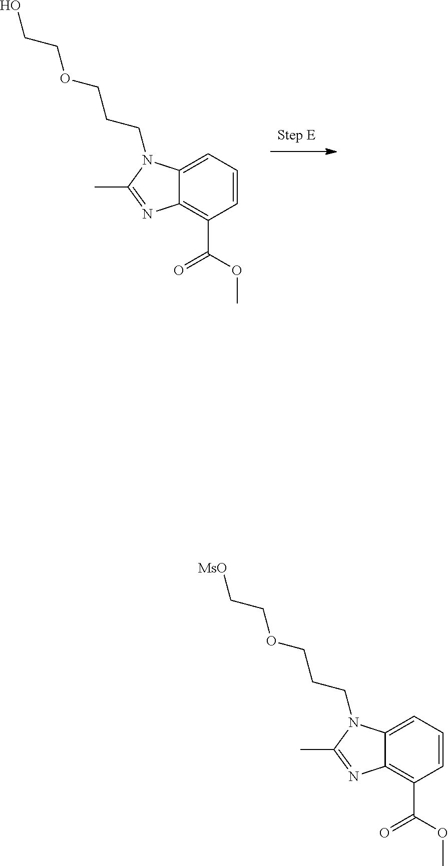 [SMILES: COC(=O)c1cccc2c1nc(C)n2CCCOCCO ; COC(=O)c1cccc2c1nc(C)n2CCCOCCOS(C)(=O)=O]

To a solution of methyl 1-(3-(2-hydroxyethoxy)propyl)-2-methyl-1H-benzo[d]imidazole-4-carboxylate (500 mg, crude) in DCM (10 mL) was added MsCl (0.132 mL, 1.71 mmol) and triethyl amine (0.286 mL, 2.052 mmol) at 0° C. under nitrogen. The reaction mixture was then allowed to stir at ambient temperature for 2 h. After complete consumption of the starting material (monitored by LCMS) the excess MsCl was quenched with saturated NaHCO₃ solution, extracted with DCM (2 times) and washed successively with water and brine. The combined organic layer was dried over Na₂SO₄ and evaporated under reduced pressure to get the crude compound which was purified by column chromatography (SiO₂, 3% MeOH in DCM) to obtain methyl 2-methyl-1-(3-(2-((methylsulfonyl)oxy)ethoxy)propyl)-1H-benzo[d]imidazole-4-carboxylate (230.0 mg, 0.621 mmol, 33% over 4 steps) as gummy liquid.

LCMS (ESI+): m/z 371.1 [M+H]⁺

### Step F

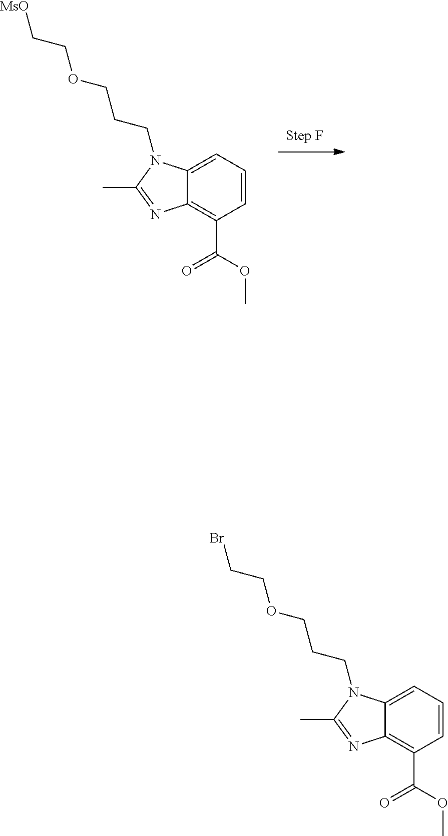 [SMILES: COC(=O)c1cccc2c1nc(C)n2CCCOCCOS(C)(=O)=O ; COC(=O)c1cccc2c1nc(C)n2CCCOCCBr]

To a solution of methyl 2-methyl-1-(3-(2-((methylsulfonyl)oxy)ethoxy)propyl)-1H-benzo[d]imidazole-4-carboxylate (230.0 mg, 0.621 mmol) in DMF (10.0 mL) was added LiBr (269 mg, 3.105 mmol) at room temperature under nitrogen. The reaction mixture was allowed to stir at 80° C. for 2 h. After complete consumption of the starting material (monitored by LCMS) the reaction mixture was then diluted with EtOAc, washed thoroughly with cold water and brine. The organic layer was dried over anhydrous Na₂SO₄ and evaporated under reduced pressure to get crude which was then purified by column chromatography (SiO₂, 2% MeOH in DCM) to get methyl 1-(3-(2-bromoethoxy)propyl)-2-methyl-1H-benzo[d]imidazole-4-carboxylate (120.0 mg, 0.338 mmol, 54%) as pale brown solid.

LCMS (ESI+): m/z 355.4 [M+H]⁺

### Step G

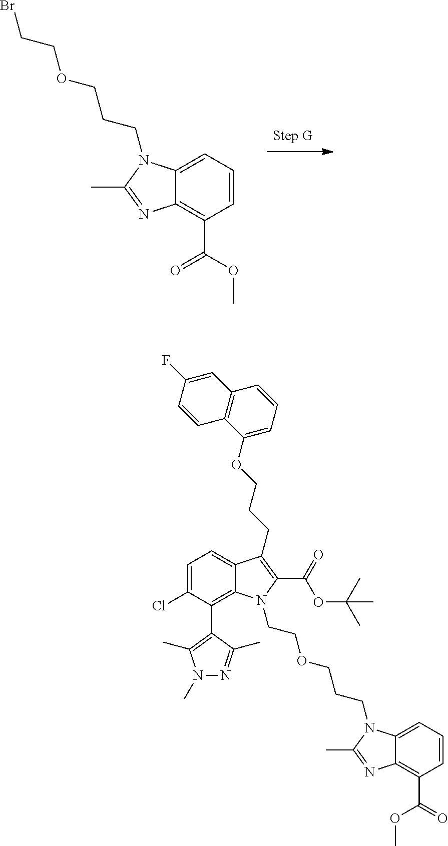 [SMILES: COC(=O)c1cccc2c1nc(C)n2CCCOCCBr ; COC(=O)c1cccc2c1nc(C)n2CCCOCCn1c(C(=O)OC(C)(C)C)c(CCCOc2cccc3cc(F)ccc23)c2ccc(Cl)c(-c3c(C)nn(C)c3C)c21]

To a solution of tert-butyl 6-chloro-3-(3-((6-fluoronaphthalen-1-yl)oxy)propyl)-7-(1,3,5-trimethyl-1H-pyrazol-4-yl)-1H-indole-2-carboxylate (125 mg, 0.223 mmol) in DMF (3 mL) was added methyl 1-(3-(2-bromoethoxy)propyl)-2-methyl-1H-benzo[d]imidazole-4-carboxylate (118.727 mg, 0.334 mmol) followed by Cs₂CO₃ (162.656 mg, 0.446 mmol) and KI (36.988 mg, 0.223 mmol) and the mixture was allowed to stir at 90° C. for 16 h under nitrogen. After complete consumption of the starting material (monitored by LCMS), the reaction mixture was diluted with EtOAc, washed thoroughly with cold water and brine. Then the organic layer was evaporated to dryness to get crude which was purified by column chromatography (SiO₂, 2% MeOH in DMC) to afford methyl 1-(3-(2-(2-(tert-butoxycarbonyl)-6-chloro-3-(3-((6-fluoronaphthalen-1-yl)oxy)propyl)-7-(1,3,5-trimethyl-1H-pyrazol-4-yl)-1H-indol-1-yl)ethoxy)propyl)-2-methyl-1H-benzo[d]imidazole-4-carboxylate (100 mg, 0.12 mmol, 54%) as white solid.

LCMS (ESI+): m/z 836.2 [M+H]⁺

### Step H

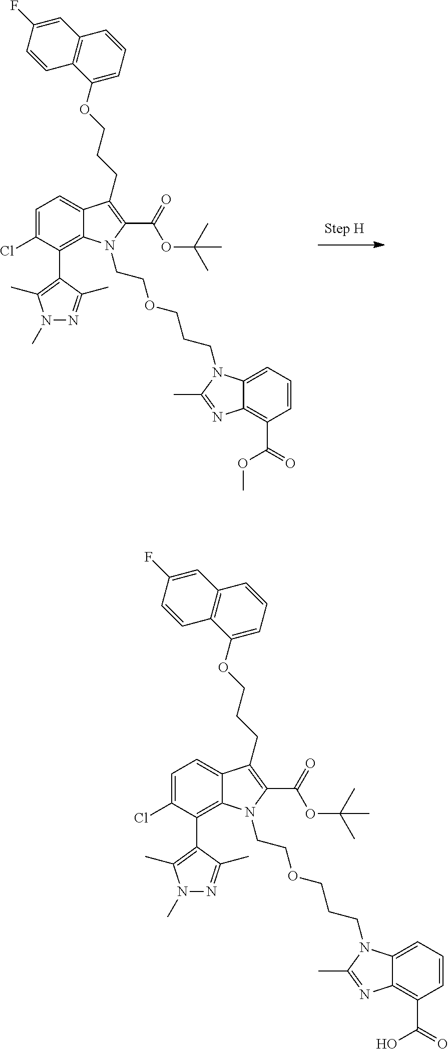 [SMILES: COC(=O)c1cccc2c1nc(C)n2CCCOCCn1c(C(=O)OC(C)(C)C)c(CCCOc2cccc3cc(F)ccc23)c2ccc(Cl)c(-c3c(C)nn(C)c3C)c21 ; Cc1nn(C)c(C)c1-c1c(Cl)ccc2c(CCCOc3cccc4cc(F)ccc34)c(C(=O)OC(C)(C)C)n(CCOCCCn3c(C)nc4c(C(=O)O)cccc43)c12]

Methyl 1-(3-(2-(2-(tert-butoxycarbonyl)-6-chloro-3-(3-((6-fluoronaphthalen-1-yl)oxy)propyl)-7-(1,3,5-trimethyl-1H-pyrazol-4-yl)-1H-indol-1-yl)ethoxy)propyl)-2-methyl-1H-benzo[d]imidazole-4-carboxylate (70.0 mg, 0.084 mmol) was dissolved in THF:H₂O (3:1; 1.5 mL) and LiOH (17.581 mg, 0.419 mmol) followed by drop of MeOH was added at 0° C. Then the reaction mass was stirred at room temperature for 16 h. After complete consumption of the starting material (monitored by LCMS) solvents were evaporated under reduced pressure to get the crude reaction mixture. It was then diluted with water and washed with EtOAc. Aqueous layer was carefully acidified using 1M HCl to pH=3, extracted with EtOAc, dried over Na₂SO₄ and organic layer was concentrated in vacuo to obtain 1-[3-(2-{2-[(tert-butoxy)carbonyl]-6-chloro-3-{3-[(6-fluoronaphthalen-1-yl)oxy]propyl}-7-(1,3,5-trimethyl-1H-pyrazol-4-yl)-1H-indol-1-yl}ethoxy)propyl]-2-methyl-1H-1,3-benzodiazole-4-carboxylic acid (35.0 mg, 0.043 mmol, 51%) as white solid.

LCMS (ESI+): m/z 822.4 [M+H]⁺

### Step I

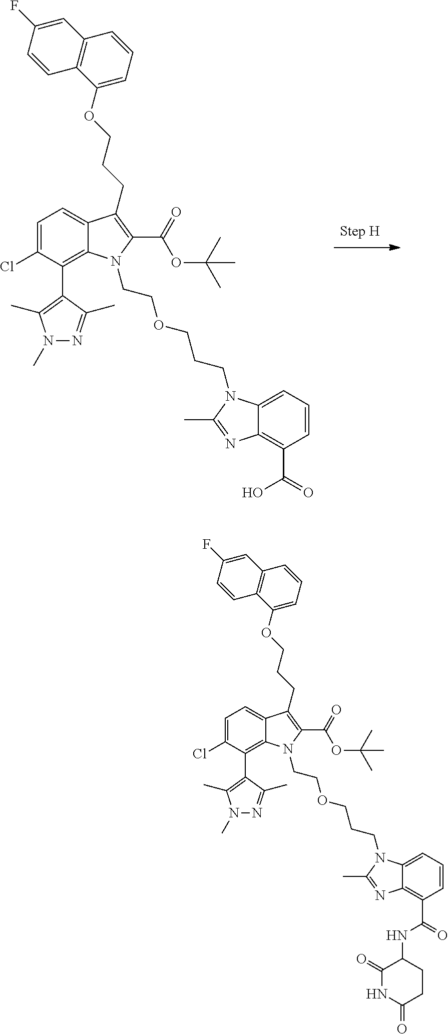 [SMILES: Cc1nn(C)c(C)c1-c1c(Cl)ccc2c(CCCOc3cccc4cc(F)ccc34)c(C(=O)OC(C)(C)C)n(CCOCCCn3c(C)nc4c(C(=O)O)cccc43)c12 ; Cc1nn(C)c(C)c1-c1c(Cl)ccc2c(CCCOc3cccc4cc(F)ccc34)c(C(=O)OC(C)(C)C)n(CCOCCCn3c(C)nc4c(C(=O)NC5CCC(=O)NC5=O)cccc43)c12]

To a solution of 1-[3-(2-{2-[(tert-butoxy)carbonyl]-6-chloro-3-{3-[(6-fluoronaphthalen-1-yl)oxy]propyl}-7-(1,3,5-trimethyl-1H-pyrazol-4-yl)-1H-indol-1-yl}ethoxy)propyl]-2-methyl-1H-1,3-benzodiazole-4-carboxylic acid (38.6 mg, 0.047 mmol), 3-aminopiperidine-2,6-dione hydrochloride (11.6 mg, 0.070 mmol) and HATU (35.6 mg, 0.094 mmol) in dry DMF (2.0 mL) was added DIPEA (0.024 mL, 0.141 mmol). The mixture was stirred at room temperature for 45 min. The crude was diluted in DCM and washed with brine. The organic layer was dried over MgSO₄, filtered and concentrated in vacuo to obtain tert-butyl 6-chloro-1-[2-(3-{4-[(2,6-dioxopiperidin-3-yl)carbamoyl]-2-methyl-1H-1,3-benzodiazol-1-yl}propoxy)ethyl]-3-{3-[(6-fluoronaphthalen-1-yl)oxy]propyl}-7-(1,3,5-trimethyl-1H-pyrazol-4-yl)-1H-indole-2-carboxylate (51 mg, crude), which was used to next step without further purification.

LCMS (ESI+): m/z 933.1 [M+H]⁺

### Step J

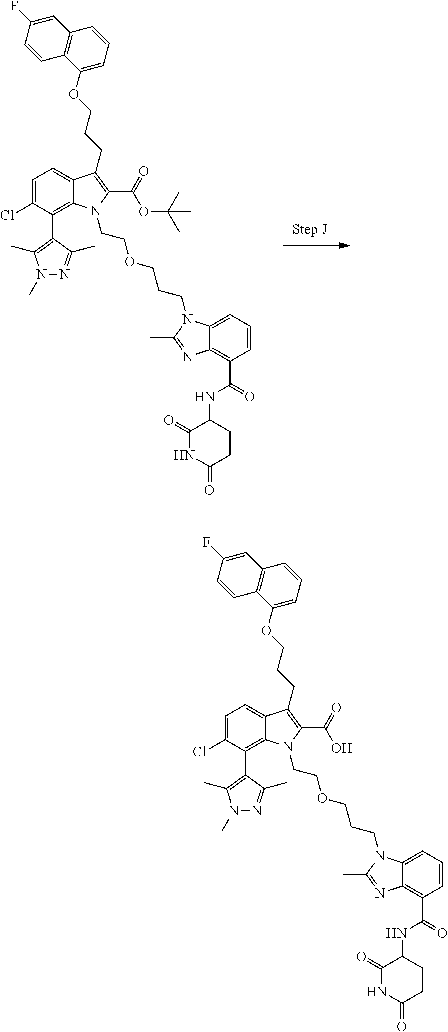 [SMILES: Cc1nn(C)c(C)c1-c1c(Cl)ccc2c(CCCOc3cccc4cc(F)ccc34)c(C(=O)OC(C)(C)C)n(CCOCCCn3c(C)nc4c(C(=O)NC5CCC(=O)NC5=O)cccc43)c12 ; Cc1nn(C)c(C)c1-c1c(Cl)ccc2c(CCCOc3cccc4cc(F)ccc34)c(C(=O)O)n(CCOCCCn3c(C)nc4c(C(=O)NC5CCC(=O)NC5=O)cccc43)c12]

To a solution of tert-butyl 6-chloro-1-[2-(3-{4-[(2,6-dioxopiperidin-3-yl)carbamoyl]-2-methyl-1H-1,3-benzodiazol-1-yl}propoxy)ethyl]-3-{3-[(6-fluoronaphthalen-1-yl)oxy]propyl}-7-(1,3,5-trimethyl-1H-pyrazol-4-yl)-1H-indole-2-carboxylate (51 mg, crude) in DCM (1.0 mL) was added TFA (1.0 mL). The mixture was stirred at room temperature for 18 h. The crude was purified with preparative HPLC (C18, H₂O:MeCN+0.1% FA) to obtain 6-chloro-1-[2-(3-{4-[(2,6-dioxopiperidin-3-yl)carbamoyl]-2-methyl-1H-1,3-benzodiazol-1-yl}propoxy)ethyl]-3-{3-[(6-fluoronaphthalen-1-yl)oxy]propyl}-7-(1,3,5-trimethyl-1H-pyrazol-4-yl)-1H-indole-2-carboxylic acid (20.0 mg, 0.023 mmol, 49% over 2 steps) as white solid.

LCMS (ESI+): m/z 876.15 [M+H]⁺

¹H NMR (500 MHz, DMSO) δ 13.35 (s, 1H), 10.90 (s, 1H), 10.17 (d, J=7.3 Hz, 1H), 8.17-8.10 (m, 1H), 7.77-7.71 (m, 2H), 7.61 (dd, J=10.4, 2.6 Hz, 1H), 7.42-7.25 (m, 4H), 7.10 (d, J=8.0 Hz, 1H), 6.89 (td, J=7.8, 1.8 Hz, 1H), 6.71 (d, J=7.5 Hz, 1H), 4.86 (ddd, J=12.6, 7.3, 5.3 Hz, 1H), 4.43-4.27 (m, 2H), 4.13 (t, J=6.3 Hz, 2H), 4.02 (t, J=6.7 Hz, 2H), 3.72 (s, 3H), 3.26-3.21 (m, 2H), 3.02 (t, J=5.4 Hz, 2H), 2.99-2.89 (m, 2H), 2.81 (ddd, J=17.3, 13.4, 5.5 Hz, 1H), 2.59-2.50 (m, 1H), 2.40 (s, 3H), 2.25-2.03 (m, 4H), 1.95 (d, J=1.5 Hz, 3H), 1.86 (s, 3H), 1.77-1.68 (m, 2H).

### Example 129. 6-Chloro-1-(2-(4-(2-((4-((2,6-dioxopiperidin-3-yl)carbamoyl)-1,2-dimethyl-1H-benzo[d]imidazol-6-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (283)

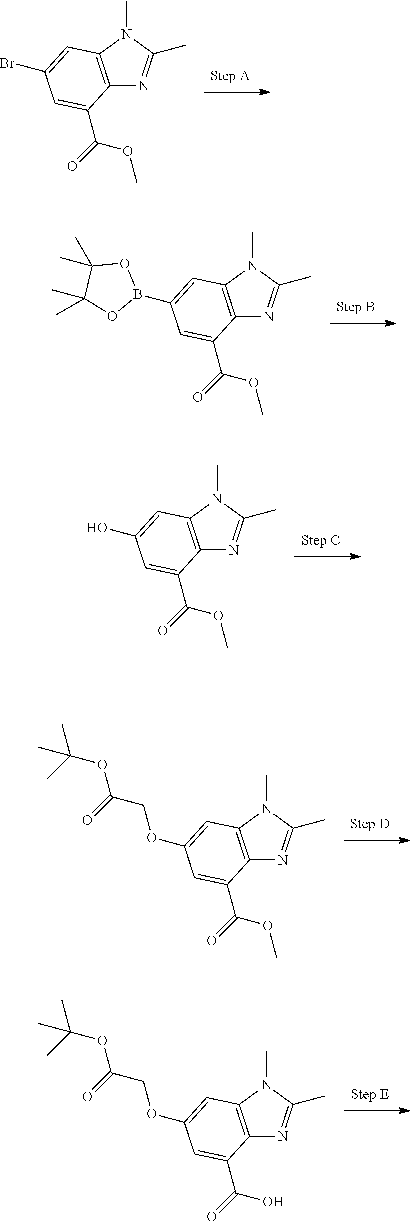

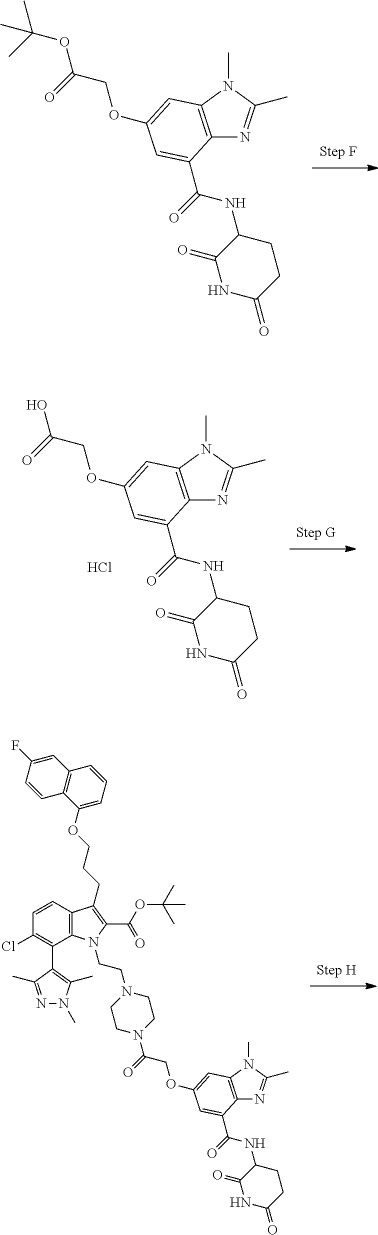

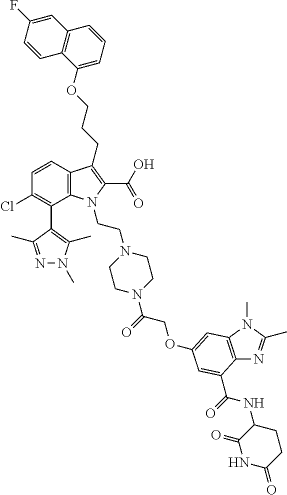

### Step A

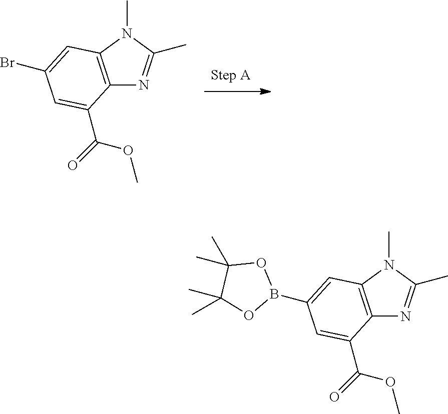

To a well solution of methyl 6-bromo-1,2-dimethyl-1H-benzo[d]imidazole-4-carboxylate (1.1 g, 3.9 mmol) in dioxane (20 mL) was added bis(pinacolato)diboron (2 g, 7.9 mmol) and KOAc (1.2 g, 12.2 mmol) successively at RT. The reaction mixture was deoxygenated with argon and to it was added Pd(dppf)Cl₂ (0.426 g, 0.58 mmol). The resulting mixture was then allowed to stir at 90° C. for 16 h. After complete consumption of the starting material (monitored by TLC and LCMS), the reaction mixture was diluted with EtOAc and washed with water and brine. The organic layer was dried over Na₂SO₄ and evaporated under reduced pressure to get the crude methyl 1,2-dimethyl-6-(4,4,5,5-tetramethyl-1,3,2-dioxaborolan-2-yl)-1H-benzo[d]imidazole-4-carboxylate (1.4 g) as light brown sticky solid which was then directly used in the next step without any purification.

LCMS (ESI+): m/z 331.3 [M+H]⁺

### Step B

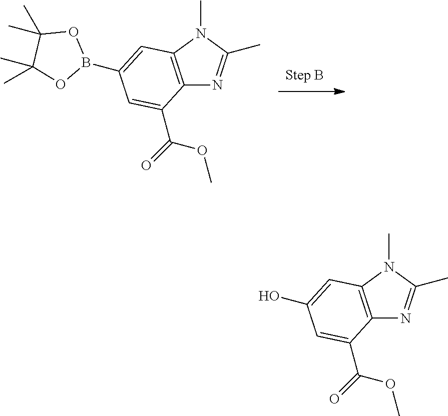

To a solution of crude methyl 1,2-dimethyl-6-(4,4,5,5-tetramethyl-1,3,2-dioxaborolan-2-yl)-1H-benzo[d]imidazole-4-carboxylate (1.4 g) in MeOH (10 mL) and water (5 mL) was added m-CPBA (1.12 g, 4.49 mmol) at 0° C. The resulting reaction mixture was stirred at room temperature for 6 h. After complete consumption of the starting material (monitored by TLC and LCMS), the reaction mixture was concentrated under reduced pressure. It was then diluted with EtOAc and washed with saturated NaHCO₃ solution, water and brine. The organic layer was dried over anhydrous Na₂SO₄ and evaporated under reduced pressure. Crude product was purified with column chromatography (SiO₂, 30-40% EtOAc in n-hexane) to get methyl 6-hydroxy-1,2-dimethyl-1H-benzo[d]imidazole-4-carboxylate (0.13 g, 0.59 mmol, 15% over 2 steps) as off white solid.

LCMS (ESI+): m/z 221.0 [M+H]⁺.

### Step C

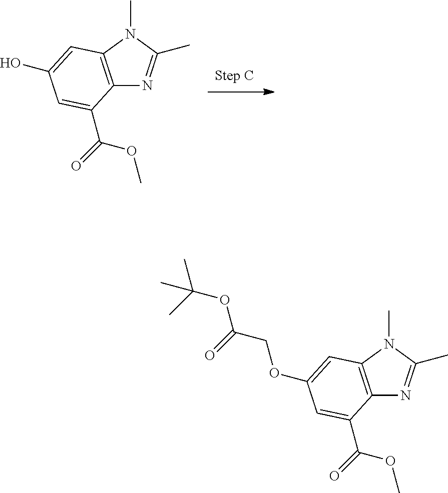 [SMILES: COC(=O)c1cc(O)cc2c1nc(C)n2C ; COC(=O)c1cc(OCC(=O)OC(C)(C)C)cc2c1nc(C)n2C]

To a well stirred solution of methyl 6-hydroxy-1,2-dimethyl-1H-benzo[d]imidazole-4-carboxylate (131 mg, 0.59 mmol) in DMF (3 mL) was added KI (30 mg, 0.18 mmol), K₂CO₃ (205 mg, 1.48 mmol) and tert-butyl 2-bromoacetate (0.096 mL, 0.654 mmol) at room temperature under nitrogen. The reaction mixture was allowed to stir at 60° C. for 1 h. After complete consumption of the starting material (monitored by TLC and LCMS), the reaction mixture was diluted with EtOAc and washed with water and brine. The organic layer was dried over Na₂SO₄ and evaporated under reduced pressure to get the crude compound, which was then purified by column chromatography (SiO₂, 40% EtOAc in DCM) to get methyl 6-(2-(tert-butoxy)-2-oxoethoxy)-1,2-dimethyl-1H-benzo[d]imidazole-4-carboxylate (170 mg, 0.51 mmol, 86%) as off white solid.

LCMS (ESI+): m/z 334.8 [M+H]⁺.

### Step D

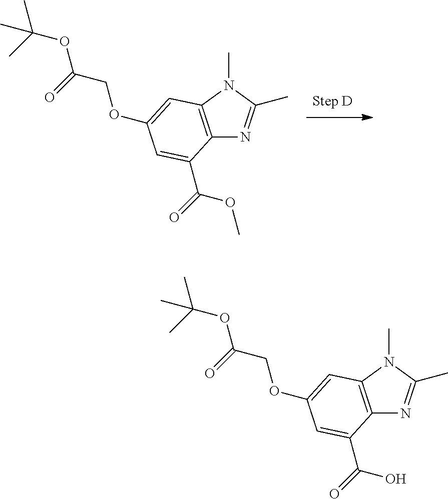

To a solution methyl 6-(2-(tert-butoxy)-2-oxoethoxy)-1,2-dimethyl-1H-benzo[d]imidazole-4-carboxylate (170 mg, 0.51 mol) in H₂O (0.5 mL) and MeCN (5 mL) were added LiBr (1.33 g, 15.3 mmol) and Et₃N (1.42 mL, 10.18 mmol) at room temperature and the mixture was stirred for 24 h. After complete consumption of the starting material (monitored by TLC and LCMS), the crude was concentrated under vacuum at ambient temperature. The residue was dissolved in water, carefully acidified to pH=˜6 at 0° C. and directly submitted to purification with reverse phase preparative HPLC (C18, 20 mM ammonium bicarbonate in H₂O:MeCN) to get 6-(2-(tert-butoxy)-2-oxoethoxy)-1,2-dimethyl-1H-benzo[d]imidazole-4-carboxylic acid (53 mg, 0.16 mmol, 31%) as off white solid.

LCMS (ESI+): m/z 321.2 [M+H]⁺.

### Step E

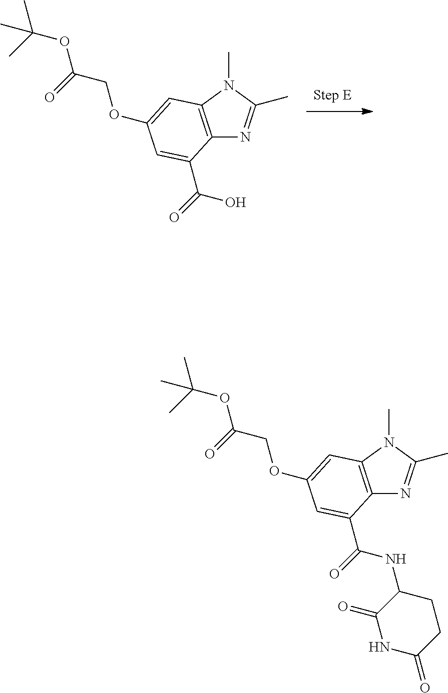

6-[2-(tert-Butoxy)-2-oxoethoxy]-1,2-dimethyl-1H-1,3-benzodiazole-4-carboxylic acid (25.0 mg, 0.078 mmol), 3-aminopiperidine-2,6-dione hydrochloride (15.4 mg, 0.094 mmol) and HATU (59.3 mg, 0.156 mmol) were dissolved in dry DMF (0.976 mL) under argon atmosphere. To it, DIPEA (0.041 mL, 0.234 mmol) was added and the reaction (monitored with LCMS) was stirred for 15 min under argon at room temperature. After complete conversion of the starting material, the reaction was concentrated under reduced pressure and the resulting residue dissolved in DMSO. Crude product was purified with reverse phase flash chromatography (C18, H₂O:MeCN+0.1% FA) to afford tert-butyl 2-({4-[(2,6-dioxopiperidin-3-yl)carbamoyl]-1,2-dimethyl-1H-1,3-benzodiazol-6-yl}oxy)acetate (15.2 mg, 0.035 mmol, 45.3%) as off white solid.

LCMS (ESI+): m/z 431.2 [M+H]⁺.

### Step F

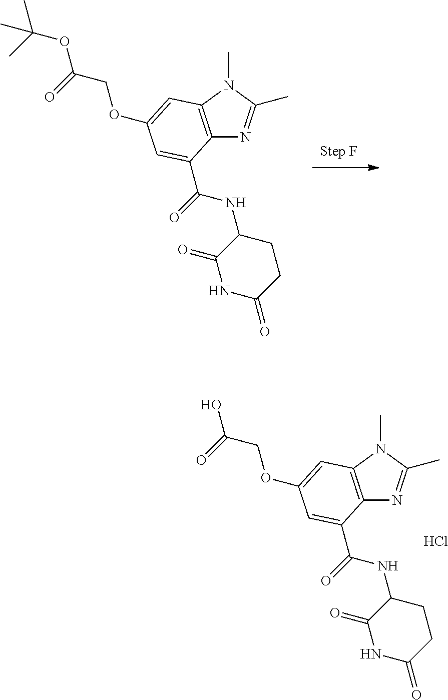 [SMILES: Cc1nc2c(C(=O)NC3CCC(=O)NC3=O)cc(OCC(=O)OC(C)(C)C)cc2n1C ; Cc1nc2c(C(=O)NC3CCC(=O)NC3=O)cc(OCC(=O)O)cc2n1C ; Cl]

tert-Butyl 2-({4-[(2,6-dioxopiperidin-3-yl)carbamoyl]-1,2-dimethyl-1H-1,3-benzodiazol-6-yl}oxy)acetate (15.2 mg, 0.035 mmol) was dissolved in dry DCM (0.400 mL) under argon atmosphere and TFA (0.400 mL, 5.224 mmol) was added. Reaction (monitored with LCMS), was stirred for 4 h at room temperature. After complete conversion of the starting material, the volatiles were evaporated and to the resulting residue an aqueous solution of 1 M HCl (1 mL) was added. The mixture was then concentrated to dryness under reduced pressure. The addition and evaporation of 1M HCl was repeated 2 times. Resulting product was freeze-dried to give 2-({4-[(2,6-dioxopiperidin-3-yl)carbamoyl]-1,2-dimethyl-1H-1,3-benzodiazol-6-yl}oxy)acetic acid hydrochloride (12.6 mg, 0.031 mmol, 86.9%) as pale white solid.

LCMS (ESI+): m/z 375.0 [M+H]⁺

### Step G

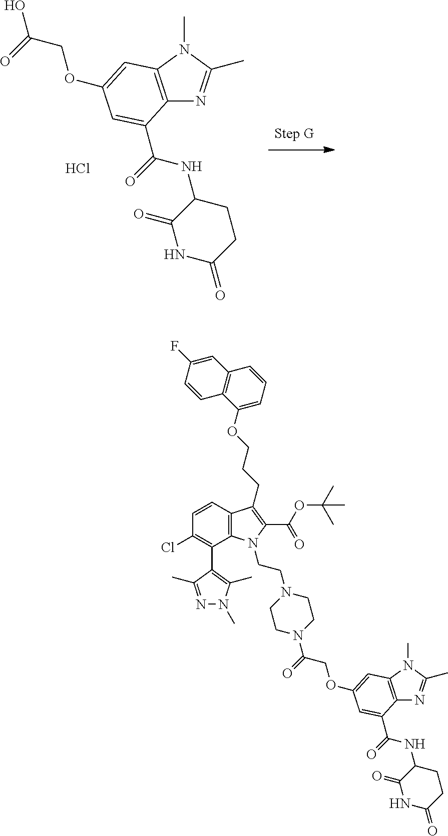

tert-Butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (15.0 mg, 0.022 mmol) and 2-({4-[(2,6-dioxopiperidin-3-yl)carbamoyl]-1,2-dimethyl-1H-1,3-benzodiazol-6-yl}oxy)acetic acid hydrochloride (11.0 mg, 0.027 mmol) were dissolved in dry DMF (1.1 mL) under argon atmosphere and DIPEA (0.012 mL, 0.067 mmol) followed by HATU (8.9 mg, 0.023 mmol) was added. Reaction (monitored with LCMS), was stirred for 15 min under argon at room temperature. After complete conversion of the starting material, the solution was diluted with DCM and washed with brine and water. The organic layer was dried over anhydrous MgSO₄, filtered and dried under reduced pressure to give crude tert-butyl 6-chloro-1-(2-{4-[2-({4-[(2,6-dioxopiperidin-3-yl)carbamoyl]-1,2-dimethyl-1H-1,3-benzodiazol-6-yl}oxy)acetyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate (29.1 mg) as yellow oil. Crude product was directly used in the next step.

LCMS (ESI+): m/z 1030.7 [M+H]⁺

### Step H

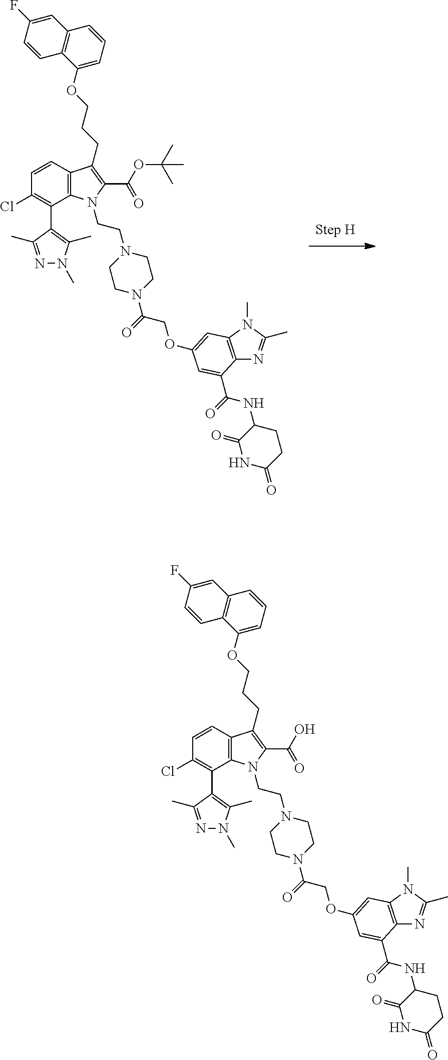

Crude tert-butyl 6-chloro-1-(2-{4-[2-({4-[(2,6-dioxopiperidin-3-yl)carbamoyl]-1,2-dimethyl-1H-1,3-benzodiazol-6-yl}oxy)acetyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate (29.1 mg) was dissolved in dry DCM (0.400 mL) under argon atmosphere and TFA (0.401 mL, 5.240 mmol) was added. Reaction (monitored with LCMS), was stirred under argon for 16 h at room temperature. After complete conversion of the starting material, the solution was concentrated under reduced pressure and dissolved in DMSO. Crude product was purified with preparative HPLC (C18, H₂O:MeCN+0.1% FA) to give 6-chloro-1-(2-(4-(2-((4-((2,6-dioxopiperidin-3-yl)carbamoyl)-1,2-dimethyl-1H-benzo[d]imidazol-6-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (9.7 mg, 0.010 mmol, 45% over 2 steps) as white solid.

LCMS (ESI+): m/z 974.2 [M+H]⁺

¹H NMR (500 MHz, DMSO, 353 K) δ 10.57 (s, 1H), 10.13 (d, J=7.2 Hz, 1H), 8.24 (dd, J=9.3, 5.8 Hz, 1H), 7.69 (d, J=8.5 Hz, 1H), 7.58 (dd, J=10.4, 2.6 Hz, 1H), 7.46 (d, J=2.5 Hz, 1H), 7.44-7.38 (m, 2H), 7.35-7.29 (m, 1H), 7.28 (d, J=2.5 Hz, 1H), 7.20 (d, J=8.5 Hz, 1H), 6.87 (dd, J=5.6, 3.0 Hz, 1H), 4.83 (ddd, J=12.3, 7.2, 5.2 Hz, 1H), 4.80 (s, 2H), 4.29 (ddd, J=13.9, 7.9, 5.9 Hz, 1H), 4.24 (t, J=6.3 Hz, 2H), 4.19 (ddd, J=13.9, 8.0, 5.8 Hz, 1H), 3.75 (s, 3H), 3.74 (s, 3H), 3.43-3.37 (m, 4H), 3.27 (dd, J=8.5, 6.6 Hz, 2H), 2.79 (ddd, J=17.5, 13.1, 5.6 Hz, 1H), 2.63-2.58 (m, 1H), 2.57 (s, 3H), 2.33-2.27 (m, 1H), 2.27-2.20 (m, 2H), 2.18-2.04 (m, 7H), 2.01 (s, 3H), 1.88 (s, 3H).

### Example 130: 6-Chloro-1-{2-[4-(2-{[3-(2,6-dioxopiperidin-3-yl)-2-methylquinolin-8-yl]amino}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (284)

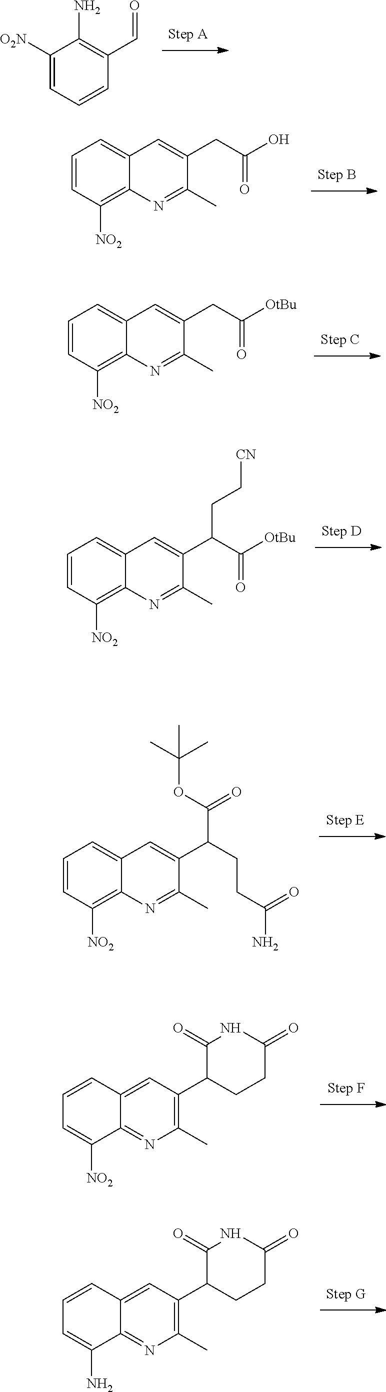

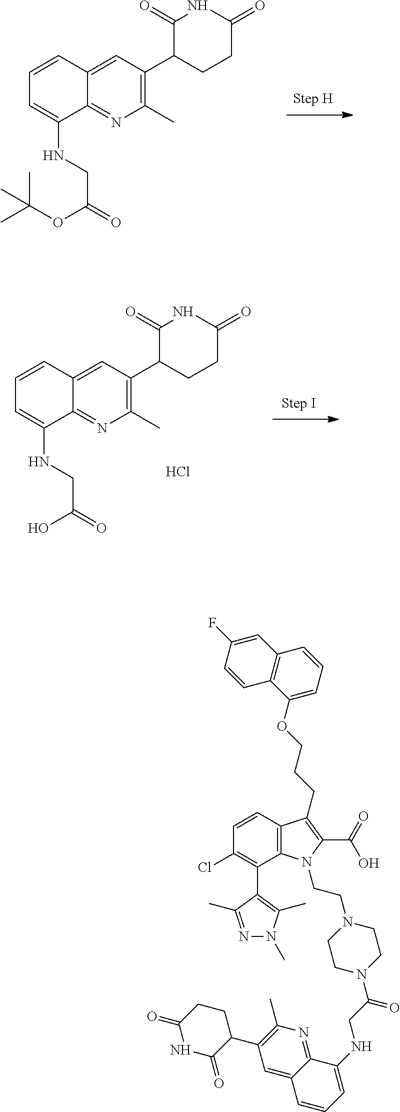 [SMILES: Cc1nc2c(NCC(=O)OC(C)(C)C)cccc2cc1C1CCC(=O)NC1=O ; Cc1nc2c(NCC(=O)O)cccc2cc1C1CCC(=O)NC1=O ; Cl ; Cc1nc2c(NCC(=O)N3CCN(CCn4c(C(=O)O)c(CCCOc5cccc6cc(F)ccc56)c5ccc(Cl)c(-c6c(C)nn(C)c6C)c54)CC3)cccc2cc1C1CCC(=O)NC1=O]

### Step A

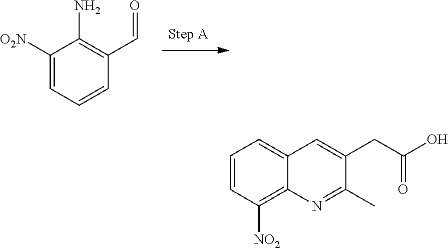 [SMILES: Nc1c(C=O)cccc1[N+](=O)[O-] ; Cc1nc2c([N+](=O)[O-])cccc2cc1CC(=O)O]

To a stirred solution of 2-amino-3-nitrobenzaldehyde (5 g, 30.041 mmol) in MeOH (50.0 mL) was added 4-oxopentanoic acid (3.4 mL, 33.045 mmol) followed by 2M aqueous sodium hydroxide solution (10.0 mL) dropwise at rt. Then the resulting reaction mixture was allowed to reflux for 16 h. After consumption of the starting material, the reaction mixture was concentrated under reduced pressure. Then it was neutralized with acetic acid and the obtained precipitate was filtered, and washed with ether and pentane to afford 2-(2-methyl-8-nitroquinolin-3-yl)acetic acid (3 g, 12.15 mmol, 40.53%).

LCMS (ESI+): m/z 247.0 [M+H]⁺

### Step B

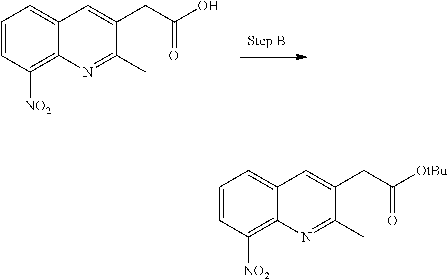 [SMILES: Cc1nc2c([N+](=O)[O-])cccc2cc1CC(=O)O ; Cc1nc2c([N+](=O)[O-])cccc2cc1CC(=O)OC(C)(C)C]

To a cooled solution of DCC (2.76 g, 13.36 mmol) in DCM (30.0 mL) was added DMAP (1.18 g, 9.72 mmol) at 0° C. followed by 2-(2-methyl-8-nitroquinolin-3-yl)acetic acid (3.0 g, 12.15 mmol) and the resulting mixture was stirred at 0° C. for 5 min. Then, tert-butanol (0.406 mL, 13.7 mmol) was added and the resulting reaction mixture was allowed to warm to room temperature for 12 h. After consumption of the starting material, the reaction mixture was diluted with EtOAc and ice-cold water. The product was extracted with EtOAc, dried over Na₂SO₄, filtered and concentrated. Crude product was purified with column chromatography (SiO₂, 25% EtOAc in hexane) to afford tert-butyl 2-(2-methyl-8-nitroquinolin-3-yl)acetate (450 mg, 1.48 mmol, 12.2%).

LCMS (ESI+): m/z 303.4 [M+H]⁺

### Step C

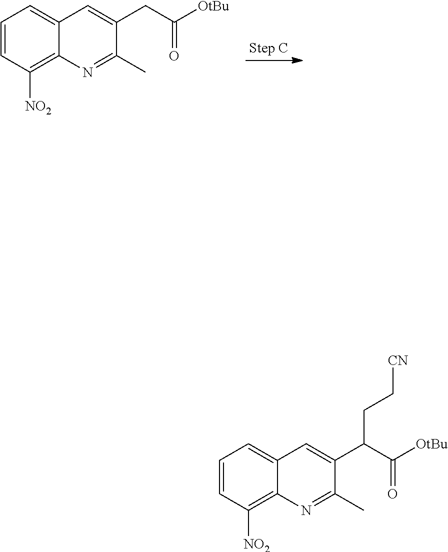 [SMILES: Cc1nc2c([N+](=O)[O-])cccc2cc1CC(=O)OC(C)(C)C ; Cc1nc2c([N+](=O)[O-])cccc2cc1C(CCC#N)C(=O)OC(C)(C)C]

To a stirred solution of tert-butyl 2-(2-methyl-8-nitroquinolin-3-yl)acetate (1.2 g, 3.97 mmol) in DMF (12 mL) were added K₂CO₃ (549 mg, 3.97 mmol) followed by addition of TEBAC (905 mg, 3.97 mmol) under nitrogen atmosphere at 0° C. Then acrylonitrile (0.264 mL, 3.974 mmol) was added into reaction mixture at rt. Then the resultant reaction mixture was allowed to stir at room temperature for 16 h. After completion of starting material, the reaction mixture was diluted with EtOAc and was added ice-cold water. Then the product was extracted EtOAc, combined organic layer was dried over Na₂SO₄, filtered and concentrated to get the crude which was purified by column chromatography (SiO₂, 15-25% EtOAc in hexane) to afford tert-butyl 4-cyano-2-(2-methyl-8-nitroquinolin-3-yl)butanoate (310.0 mg, 0.87 mmol, 22%).

LCMS (ESI+): m/z 356.2 [M+H]⁺

### Step D

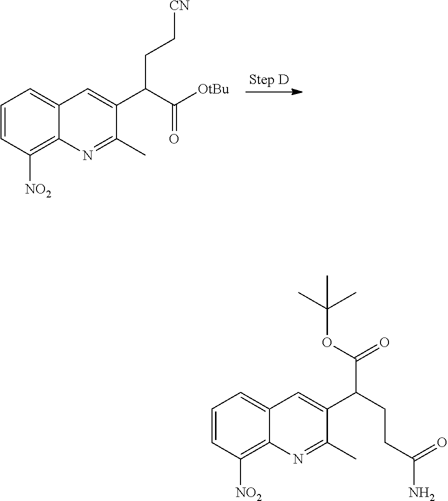 [SMILES: Cc1nc2c([N+](=O)[O-])cccc2cc1C(CCC#N)C(=O)OC(C)(C)C ; Cc1nc2c([N+](=O)[O-])cccc2cc1C(CCC(N)=O)C(=O)OC(C)(C)C]

To a stirred solution of tert-butyl 4-cyano-2-(2-methyl-8-nitroquinolin-3-yl)butanoate (310.0 mg, 0.871 mmol) in DMSO (4.0 mL) were added H₂O₂ (0.444 mL, 4.354 mmol) followed by K₂CO₃ (12.034 mg, 0.087 mmol) under nitrogen atmosphere at 0° C. Then the resultant reaction mixture was allowed stir at room temperature for 16 h. After complete conversion of the starting material, the reaction mixture was diluted with EtOAc and added ice-cold water and extracted with EtOAc. The organic layer was dried over Na₂SO₄, filtered and concentrated to get the crude product which was purified with column chromatography (SiO₂, 90% EtOAc in hexane) to afford tert-butyl 5-amino-2-(2-methyl-8-nitroquinolin-3-yl)-5-oxopentanoate (150 mg, 0.402 mmol, 46.1%).

LCMS (ESI+): m/z 374.3 [M+H]⁺

### Step E

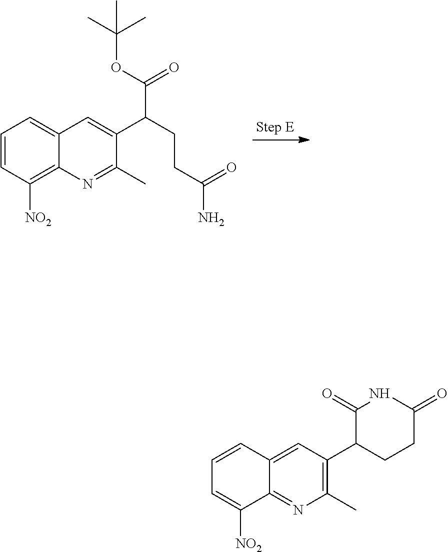 [SMILES: Cc1nc2c([N+](=O)[O-])cccc2cc1C(CCC(N)=O)C(=O)OC(C)(C)C ; Cc1nc2c([N+](=O)[O-])cccc2cc1C1CCC(=O)NC1=O]

To the reaction vial were added tert-butyl 5-amino-2-(2-methyl-8-nitroquinolin-3-yl)-5-oxopentanoate (106.0 mg, 0.284 mmol), MeCN (7.1 mL) and p-TsOH·H₂O (540.0 mg, 2.839 mmol). Vial was sealed and the reaction was carried out at 80° C. for 18 h. Crude was purified directly by reversed phase flash column chromatography (H₂O:MeCN+0.1% FA). Fractions were collected, concentrated and freeze-dried to afford 3-(2-methyl-8-nitroquinolin-3-yl)piperidine-2,6-dione (84.0 mg, 0.272 mmol, 95.9%) as yellow solid. Product was used directly in the next step.

LCMS (ESI+): m/z 300.26 [M+H]⁺

### Step F

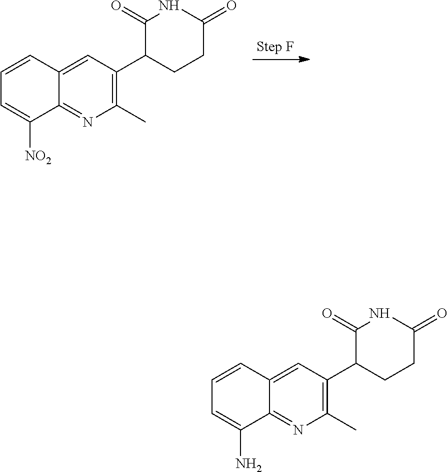 [SMILES: Cc1nc2c([N+](=O)[O-])cccc2cc1C1CCC(=O)NC1=O ; Cc1nc2c(N)cccc2cc1C1CCC(=O)NC1=O]

To the flask with 3-(2-methyl-8-nitroquinolin-3-yl)piperidine-2,6-dione (84.0 mg, 0.281 mmol) was added EtOH (9.8 mL) and the mixture was purged with argon, then Pd/C (3.0 mg) was added. Argon was displaced with H₂ and the reaction was carried out under hydrogen atmosphere for 4 h. Mixture was filtered and evaporated, then freeze-dried to give 3-(8-amino-2-methylquinolin-3-yl)piperidine-2,6-dione (79.0 mg, 0.279 mmol, 99.3%) as a yellow solid. Product was used directly in the next step.

LCMS: (ESI+) m/z 270.05 [M+H]⁺

### Step G

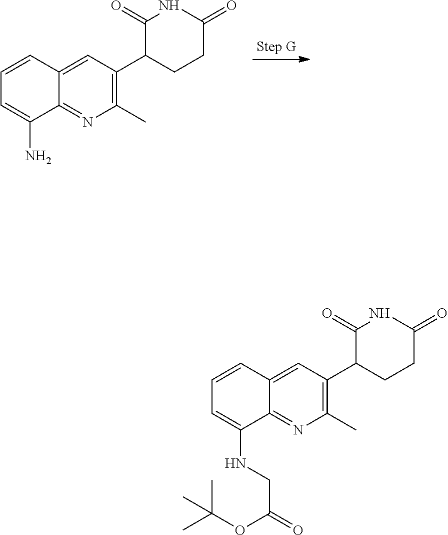 [SMILES: Cc1nc2c(N)cccc2cc1C1CCC(=O)NC1=O ; Cc1nc2c(NCC(=O)OC(C)(C)C)cccc2cc1C1CCC(=O)NC1=O]

3-(8-Amino-2-methylquinolin-3-yl)piperidine-2,6-dione (84.0 mg, 0.312 mmol) was dissolved in DMF (855 μL) and KI (51.8 mg, 0.312 mmol) and KHCO₃ (93.7 mg, 0.936 mmol) were added, followed by dropwise addition of tert-butyl bromoacetate_(46 μL, 0.312 mmol) in DMF (855 μL). Reaction was stirred in room temperature for 5 days. Reaction mixture was directly purified with reverse phase flash chromatography (C18, H₂O:MeCN+0.1% FA) to give a corresponding tert-butyl 2-{[3-(2,6-dioxopiperidin-3-yl)-2-methylquinolin-8-yl]amino}acetate (9.4 mg, 0.025 mmol, 7.9%) as a white solid. Product was used directly in the next step.

LCMS (ESI+): m/z 384.28 [M+H]⁺

### Step H

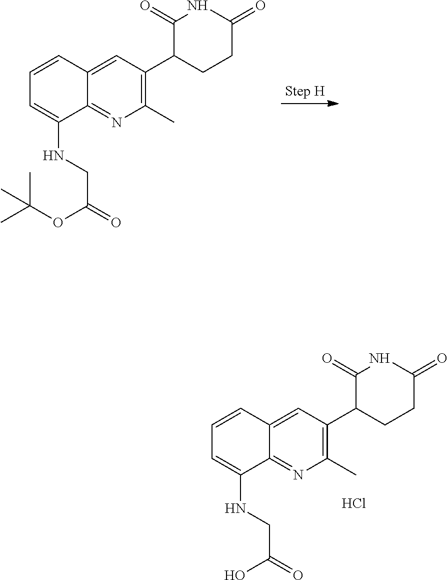 [SMILES: Cc1nc2c(NCC(=O)OC(C)(C)C)cccc2cc1C1CCC(=O)NC1=O ; Cc1nc2c(NCC(=O)O)cccc2cc1C1CCC(=O)NC1=O ; Cl]

tert-Butyl 2-{[3-(2,6-dioxopiperidin-3-yl)-2-methylquinolin-8-yl]amino}acetate (9.4 mg, 0.025 mmol) was dissolved in DCM (188 μL), and TFA (188 μL, 2.451 mmol) was added. Mixture was stirred in room temperature for overnight. Solvents were removed under reduced pressure, and product was co-evaporated 3× with 1M HCl. 2-{[3-(2,6-Dioxopiperidin-3-yl)-2-methylquinolin-8-yl]amino}acetic acid hydrochloride (8.9 mg, 0.024 mmol, 99.8%) was isolated as orange solid.

LCMS (ESI+): m/z 327.9 [M+H]⁺

### Step I

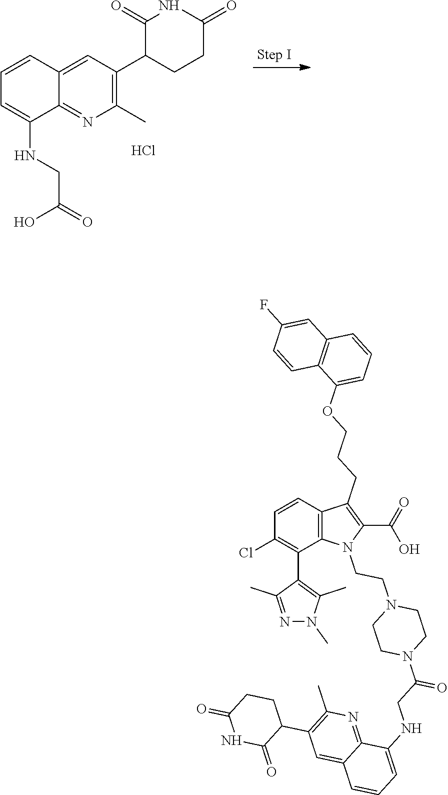 [SMILES: Cc1nc2c(NCC(=O)O)cccc2cc1C1CCC(=O)NC1=O ; Cl ; Cc1nc2c(NCC(=O)N3CCN(CCn4c(C(=O)O)c(CCCOc5cccc6cc(F)ccc56)c5ccc(Cl)c(-c6c(C)nn(C)c6C)c54)CC3)cccc2cc1C1CCC(=O)NC1=O]

2-{[3-(2,6-Dioxopiperidin-3-yl)-2-methylquinolin-8-yl]amino}acetic acid hydrochloride (8.9 mg, 0.024 mmol) was dissolved in DMF (3.1 mL), and DIPEA (0.017 mL, 0.098 mmol) was added, followed by HATU (8.7 mg, 0.023 mmol). Mixture was stirred in room temperature for 1 h then solution of 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylic acid (12.1 mg, 0.020 mmol) in DMSO (0.756 mL) was added dropwise and mixture stirred for 15 min. Crude mixture was purified directly with preparative HPLC (C18, H₂O:MeCN+0.1% FA) to give a corresponding 6-chloro-1-{2-[4-(2-{[3-(2,6-dioxopiperidin-3-yl)-2-methylquinolin-8-yl]amino}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (5.0 mg, 0.005 mmol, 22.0%) as a yellow solid.

LCMS (ESI−): m/z 925.35 [M−H]⁻

¹H NMR (500 MHz, DMSO, 353K) δ=10.56 (s, 1H), 8.24 (dd, J=9.2, 5.9, 1H), 7.92 (s, 1H), 7.72 (d, J=8.5, 1H), 7.58 (dd, J=10.4, 2.6, 1H), 7.45-7.39 (m, 2H), 7.32 (td, J=8.9, 2.6, 1H), 7.27 (t, J=7.8, 1H), 7.23 (d, J=8.5, 1H), 7.01 (dd, J=8.2, 1.2, 1H), 6.92-6.81 (m, 2H), 6.63 (dd, J=7.7, 1.2, 1H), 4.33-4.14 (m, 5H), 4.04 (s, 2H), 3.77 (s, 3H), 3.48-3.35 (m, 4H), 3.29 (dd, J=8.5, 6.5, 2H), 2.81 (ddd, J=17.6, 12.5, 5.4, 1H), 2.67 (s, 3H), 2.65-2.59 (m, 1H), 2.42-2.32 (m, 1H), 2.29-2.21 (m, 2H), 2.20-2.06 (m, 7H), 2.02 (s, 3H), 1.90 (s, 3H).

### Example 131. 6-Chloro-1-(2-(4-((3-(2,6-dioxopiperidin-3-yl)-2-methylquinolin-7-yl)glycyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (285)

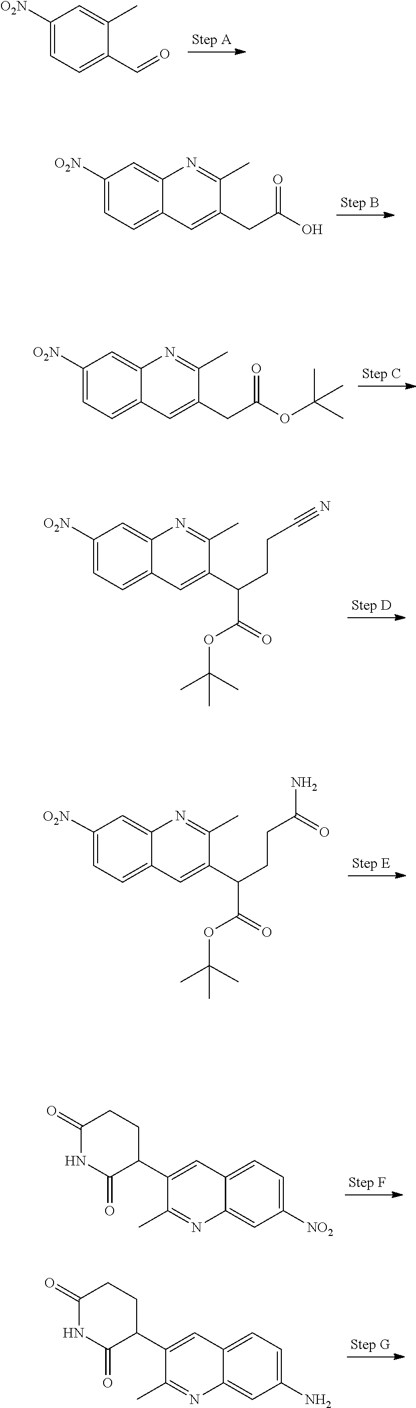

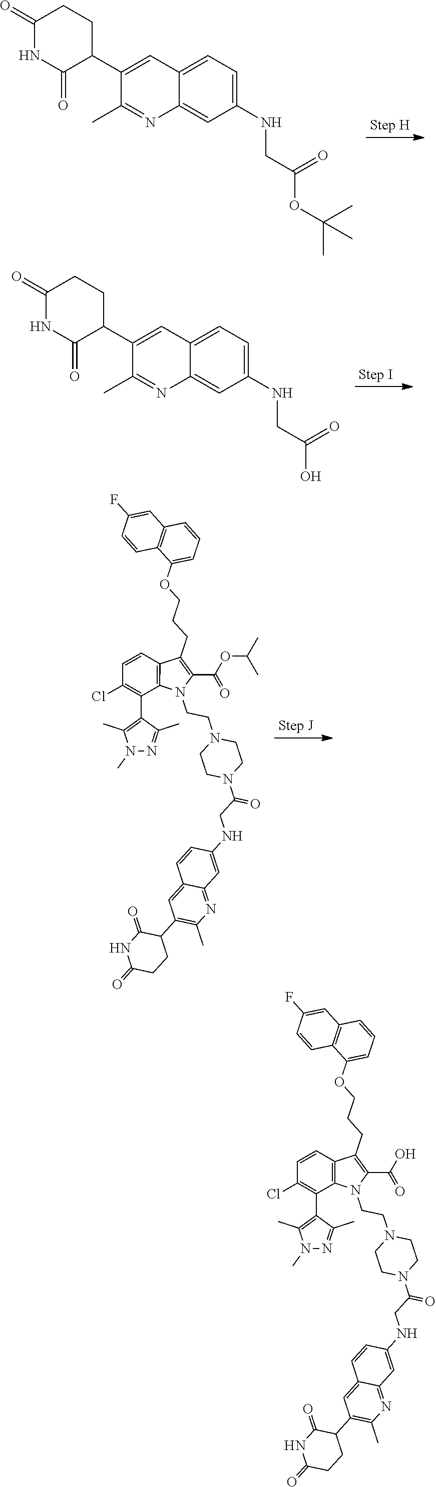

### Step A

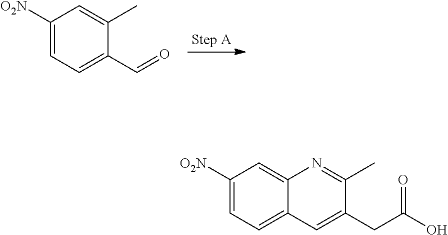 [SMILES: Cc1cc([N+](=O)[O-])ccc1C=O ; Cc1nc2cc([N+](=O)[O-])ccc2cc1CC(=O)O]

To a solution of 2-amino-4-nitrobenzaldehyde (560 mg, 3.37 mmol) in MeOH (4 mL) was added 4-oxopentanoic acid (0.35 mL, 3.37 mmol) followed by 2 M NaOH in H₂O (1 mL). The reaction mixture was refluxed for 18 h, concentrated under reduced pressure and neutralized with acetic acid. The solids were filtered and diluted with Et₂O/pentane to give 2-(2-methyl-7-nitroquinolin-3-yl)acetic acid (830 mg, 3.37 mmol, quantitative).

LCMS (ESI+): m/z 247.1 [M+H]⁺

### Step B

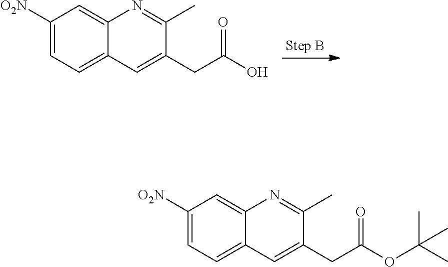 [SMILES: Cc1nc2cc([N+](=O)[O-])ccc2cc1CC(=O)O ; Cc1nc2cc([N+](=O)[O-])ccc2cc1CC(=O)OC(C)(C)C]

To a cooled solution of DCC (1.39 g, 6.72 mmol) in DCM (20 mL) was added DMAP (534 mg, 4.37 mmol) at 0° C., followed by 2-(2-methyl-7-nitroquinolin-3-yl)acetic acid (830 mg, 3.36 mmol) and the resulting mixture was stirred at 0° C. for 5 min. Then tert-butanol (0.5 mL, 16.8 mmol) was added and the reaction mixture was allowed to stir at room temperature for 16 h under nitrogen. After complete consumption of the starting material (monitored by LCMS) the solvent of reaction mixture was evaporated under reduced pressure to get the crude compound, which was diluted with EtOAc and washed successively with water and brine. Organic layer was dried over Na₂SO₄ and evaporated under reduced pressure to get the crude compound, which was then purified by column chromatography (SiO₂, 60% EtOAc in hexane) to obtain tert-butyl 2-(2-methyl-7-nitroquinolin-3-yl)acetate (460 mg, 1.52 mmol, 45%) as off white solid.

LCMS (ESI+): m/z 302.8 [M+H]⁺

### Step C

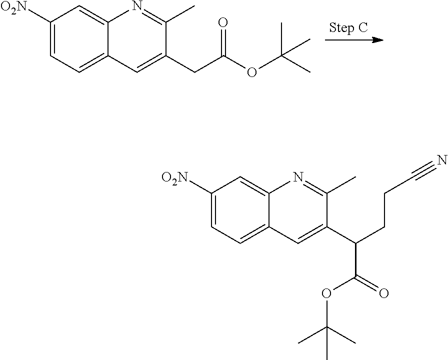 [SMILES: Cc1nc2cc([N+](=O)[O-])ccc2cc1CC(=O)OC(C)(C)C ; Cc1nc2cc([N+](=O)[O-])ccc2cc1C(CCC#N)C(=O)OC(C)(C)C]

To a stirred solution of tert-butyl 2-(2-methyl-7-nitroquinolin-3-yl)acetate (460 mg, 1.52 mmol) in DMF (10 mL) were added K₂CO₃ (210 mg, 1.52 mmol) followed by TEBAC (346.56 mg, 1.52 mmol) under nitrogen atmosphere at 0° C. Then acrylonitrile (0.1 mL, 1.52 mmol) was added into the reaction mixture at room temperature and the resultant reaction mixture was allowed to stir at room temperature for 16 h under nitrogen. After complete consumption of the starting material (monitored by TLC and LCMS) the reaction mixture was diluted with EtOAc and washed successively with cold water and brine. The organic layer was dried over Na₂SO₄ and evaporated under reduced pressure to get the crude compound, which was then purified by column chromatography (SiO₂, 50% EtOAc in hexane) to obtain tert-butyl 4-cyano-2-(2-methyl-7-nitroquinolin-3-yl)butanoate (255 mg, 0.72 mmol, 47%) as brownish liquid.

### Step D

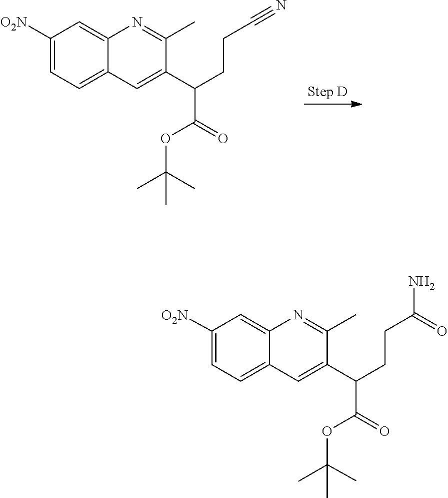 [SMILES: Cc1nc2cc([N+](=O)[O-])ccc2cc1C(CCC#N)C(=O)OC(C)(C)C ; Cc1nc2cc([N+](=O)[O-])ccc2cc1C(CCC(N)=O)C(=O)OC(C)(C)C]

To a stirred solution of tert-butyl 4-cyano-2-(2-methyl-7-nitroquinolin-3-yl)butanoate (255 mg, 0.72 mmol) in DMSO (5.0 mL) were added H₂O₂ (0.12 mL, 3.66 mmol) followed by K₂CO₃ (14 mg, 0.10 mmol) under nitrogen atmosphere at 0° C. Then the resultant reaction mixture was allowed stir at room temperature for 48 h under nitrogen. After complete consumption of the starting material (monitored by LCMS) the reaction mixture was diluted with EtOAc and washed successively with cold water and brine. The organic layer was dried over Na₂SO₄ and evaporated under reduced pressure to get the crude compound, which was then purified by column chromatography (SiO₂, 80% EtOAc in hexane) to obtain tert-butyl 5-amino-2-(2-methyl-7-nitroquinolin-3-yl)-5-oxopentanoate (95 mg, 0.254 mmol, 35%) as off white sticky solid.

LCMS (ESI+): m/z 374.2 [M+H]⁺

### Step E

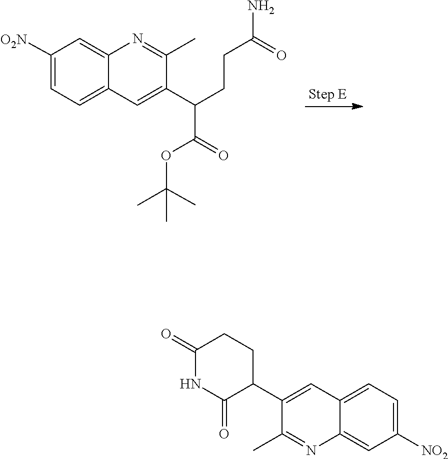 [SMILES: Cc1nc2cc([N+](=O)[O-])ccc2cc1C(CCC(N)=O)C(=O)OC(C)(C)C ; Cc1nc2cc([N+](=O)[O-])ccc2cc1C1CCC(=O)NC1=O]

To tert-butyl 5-amino-2-(2-methyl-7-nitroquinolin-3-yl)-5-oxopentanoate (65.0 mg, 0.174 mmol) dissolved in MeCN (4.3 mL) was added p-toluenesulfonic acid monohydrate (331.1 mg, 1.741 mmol). The mixture was stirred for 18 h at 80° C. in a sealed vial. To the crude was added Et₃N (0.242 mL, 1.741 mmol, the solvent was evaporated and the residues were suspended in DCM and purified by flash chromatography (SiO₂, DCM:MeOH 0 to 7%) to give corresponding 3-(2-methyl-7-nitroquinolin-3-yl)piperidine-2,6-dione (42.0 mg, 0.140 mmol, 80.4%) as a light orange solid.

LCMS (ESI+): m/z 299.9 [M+H]⁺

### Step F

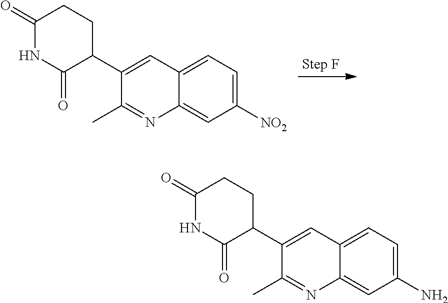 [SMILES: Cc1nc2cc([N+](=O)[O-])ccc2cc1C1CCC(=O)NC1=O ; Cc1nc2cc(N)ccc2cc1C1CCC(=O)NC1=O]

3-(2-Methyl-7-nitroquinolin-3-yl)piperidine-2,6-dione (80.0 mg, 0.267 mmol) was mixed with palladium on activated carbon (25.0 mg) and purged with argon. EtOH (15.0 mL) was added and the slurry was evacuated and backfilled with hydrogen three times. The slurry was then stirred at room temperature for 2 h under hydrogen atmosphere. LCMS indicated full conversion. The solid residues were filtered off, washed with EtOH and the collected organic filtrate was evaporated. The 3-(7-amino-2-methylquinolin-3-yl)piperidine-2,6-dione (71.4 mg, crude) was vacuum-dried and used directly in the next step.

LCMS (ESI+): m/z 270.0 [M+H]⁺

### Step G

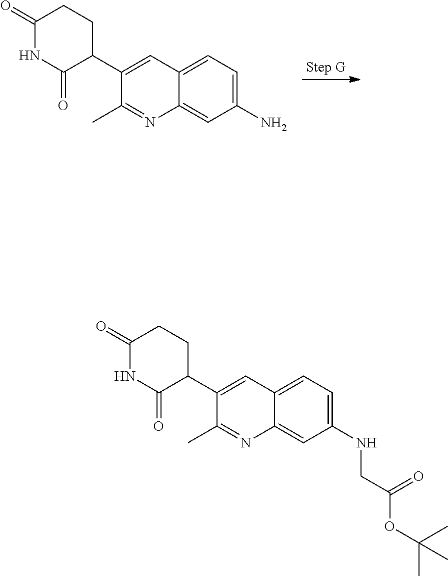 [SMILES: Cc1nc2cc(N)ccc2cc1C1CCC(=O)NC1=O ; Cc1nc2cc(NCC(=O)OC(C)(C)C)ccc2cc1C1CCC(=O)NC1=O]

The 3-(7-amino-2-methylquinolin-3-yl)piperidine-2,6-dione (71.4 mg, crude) and TBAl (0.265 mL, 0.265 mmol) were purged with argon and dissolved in dry DMF (15.0 mL). Then DIPEA (0.185 mL, 1.060 mmol) was added followed by 1M solution of tert-butyl bromoacetate in DMF (97.9 mg, 0.265 mmol). The reaction was stirred at 60° C. for 4 h and LCMS showed partial conversion. Additional portions of DIPEA (0.185 mL, 1.060 mmol) and 1M solution of tert-butyl bromoacetate in DMF (0.265 mL, 0.265 mmol) were added and stirring was prolonged for overnight at the same temperature. The crude was purified by reverse phase flash chromatography (C18, H₂O:MeCN+0.1% FA) to obtain tert-butyl (3-(2,6-dioxopiperidin-3-yl)-2-methylquinolin-7-yl)glycinate (14.0 mg, 0.037 mmol, 13.8% over two steps).

LCMS (ESI+): m/z 384.2 [M+H]⁺

### Step H

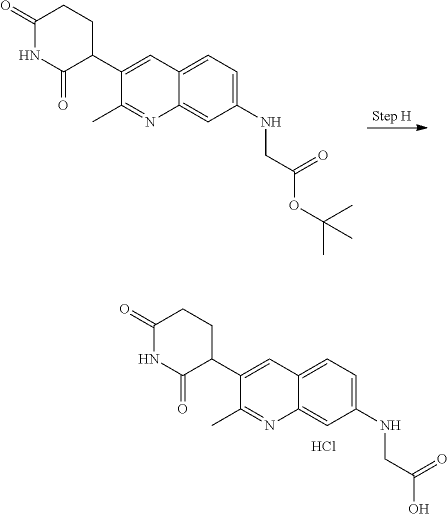 [SMILES: Cc1nc2cc(NCC(=O)OC(C)(C)C)ccc2cc1C1CCC(=O)NC1=O ; Cc1nc2cc(NCC(=O)O)ccc2cc1C1CCC(=O)NC1=O ; Cl]

The tert-butyl (3-(2,6-dioxopiperidin-3-yl)-2-methylquinolin-7-yl)glycinate (9.3 mg, 0.018 mmol) was solubilized in dioxane (1 mL) and 36% aqueous HCl solution (2.0 mL, 23.2 mmol) was added. The clear pale yellow solution was stirred at room temperature for 2 h. LCMS indicated full conversion. The solution was evaporated to obtain (3-(2,6-dioxopiperidin-3-yl)-2-methylquinolin-7-yl)glycine hydrochloride (8.0 mg, 0.024 mmol, quantitative) which was used directly for the next step.

LCMS (ESI+): m/z 328.0 [M+H]⁺

### Step I

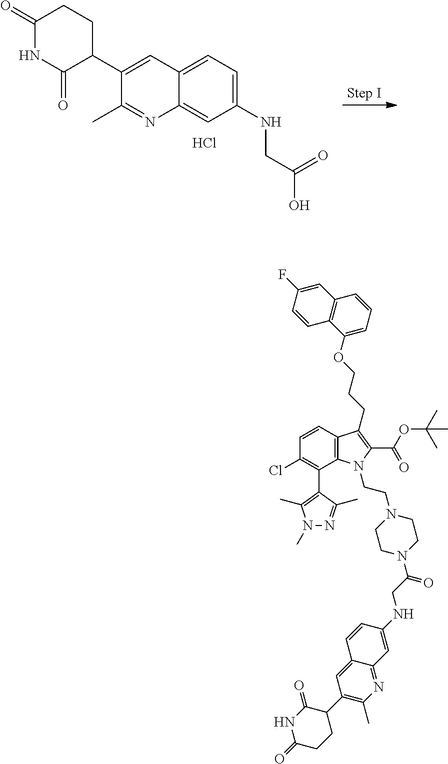 [SMILES: Cc1nc2cc(NCC(=O)O)ccc2cc1C1CCC(=O)NC1=O ; Cl ; Cc1nc2cc(NCC(=O)N3CCN(CCn4c(C(=O)OC(C)(C)C)c(CCCOc5cccc6cc(F)ccc56)c5ccc(Cl)c(-c6c(C)nn(C)c6C)c54)CC3)ccc2cc1C1CCC(=O)NC1=O]

To a solution of 2-{[3-(2,6-dioxopiperidin-3-yl)-2-methylquinolin-7-yl]amino}acetic acid hydrochloride (8.0 mg, 0.024 mmol), tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (16.3 mg, 0.024 mmol) and HATU (16.7 mg, 0.044 mmol) in dry DMF (2.0 mL) was added DIPEA (0.011 mL, 0.066 mmol). The mixture was stirred at room temperature for 30 min. The crude was diluted with DCM and washed with brine. The organic layer was dried over MgSO₄, filtered and concentrated in vacuo to get 31 mg crude tert-butyl 6-chloro-1-{2-[4-(2-{[3-(2,6-dioxopiperidin-3-yl)-2-methylquinolin-7-yl]amino}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate as yellow oil which was used to next step without further purification.

LCMS (ESI+): m/z 983.4 [M+H]⁺

### Step J

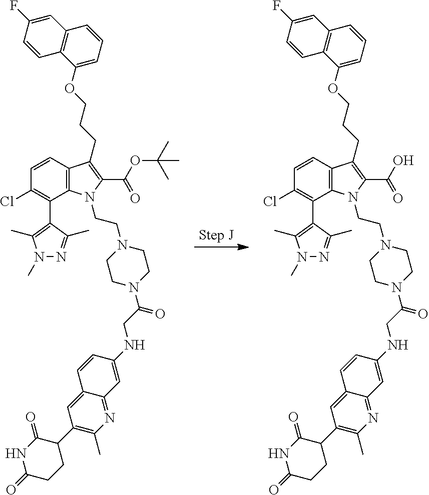 [SMILES: Cc1nc2cc(NCC(=O)N3CCN(CCn4c(C(=O)OC(C)(C)C)c(CCCOc5cccc6cc(F)ccc56)c5ccc(Cl)c(-c6c(C)nn(C)c6C)c54)CC3)ccc2cc1C1CCC(=O)NC1=O ; Cc1nc2cc(NCC(=O)N3CCN(CCn4c(C(=O)O)c(CCCOc5cccc6cc(F)ccc56)c5ccc(Cl)c(-c6c(C)nn(C)c6C)c54)CC3)ccc2cc1C1CCC(=O)NC1=O]

To a solution of tert-butyl 6-chloro-1-{2-[4-(2-{[3-(2,6-dioxopiperidin-3-yl)-2-methylquinolin-7-yl]amino}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (31 mg, crude) in DCM (1.0 mL) was added TFA (1.0 mL, 13.059 mmol). The mixture was stirred for 18 h at RT. The crude was concentrated in vacuo and purified with preparative HPLC (C18, H₂O:MeCN+0.1% FA) to obtain 6-chloro-1-{2-[4-(2-{[3-(2,6-dioxopiperidin-3-yl)-2-methylquinolin-7-yl]amino}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (7.0 mg, 0.008 mmol, 33% over two steps) as a yellow solid.

LCMS (ESI+): m/z 927.4 [M+H]⁺

¹H NMR (500 MHz, DMSO, 353 K) δ 10.51 (s, 1H), 8.24 (dd, J=9.2, 5.9 Hz, 1H), 7.75 (s, 1H), 7.71 (d, J=8.5 Hz, 1H), 7.58 (dd, J=10.4, 2.6 Hz, 1H), 7.53 (d, J=8.8 Hz, 1H), 7.44-7.40 (m, 2H), 7.36-7.28 (m, 1H), 7.22 (d, J=8.5 Hz, 1H), 7.07 (dd, J=8.8, 2.3 Hz, 1H), 6.87 (dd, J=5.4, 3.3 Hz, 2H), 6.85 (d, J=2.3 Hz, 1H), 5.92-5.83 (m, 1H), 4.33-4.28 (m, 1H), 4.25 (t, J=6.3 Hz, 2H), 4.24-4.15 (m, 1H), 4.12 (dd, J=11.8, 5.0 Hz, 1H), 3.98 (d, J=4.9 Hz, 2H), 3.77 (s, 3H), 3.45-3.39 (m, 4H), 3.32-3.25 (m, 2H), 2.78 (ddd, J=17.4, 12.3, 5.4 Hz, 1H), 2.63-2.57 (m, 1H), 2.56 (s, 3H), 2.36-2.20 (m, 3H), 2.18-2.03 (m, 7H), 2.01 (s, 3H), 1.89 (s, 3H).

### Example 132. 6-Chloro-1-(2-(4-((3-(2,6-dioxopiperidin-3-yl)-2-methylquinolin-6-yl)glycyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (286)

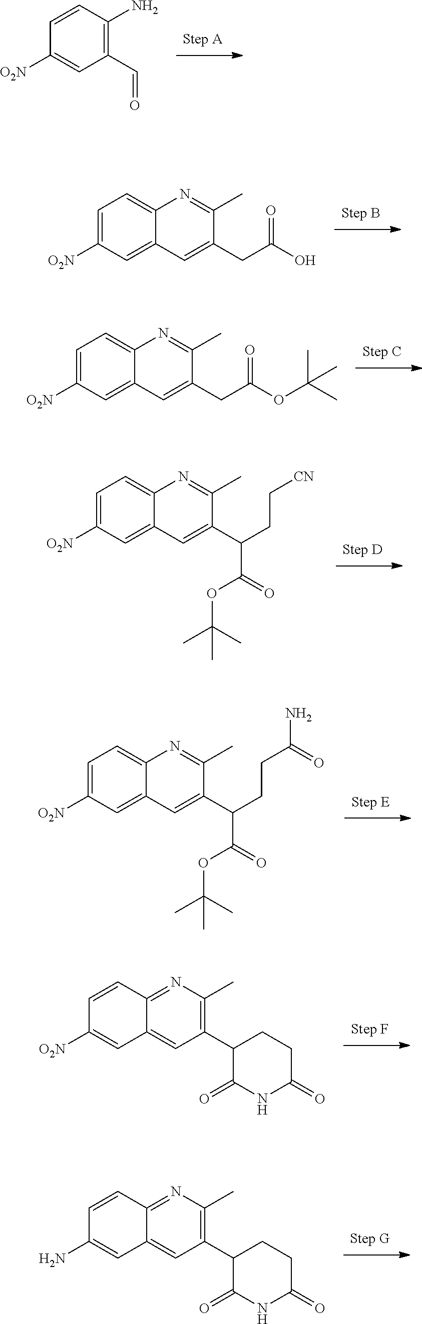

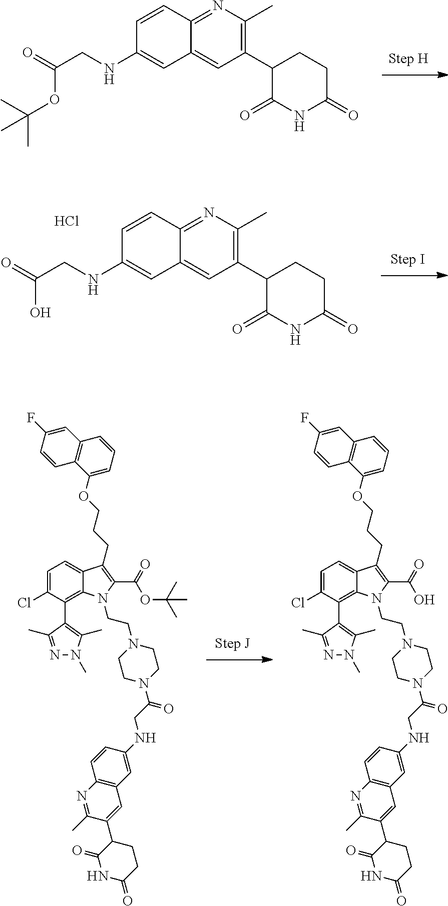 [SMILES: Cc1nc2ccc(NCC(=O)OC(C)(C)C)cc2cc1C1CCC(=O)NC1=O ; Cc1nc2ccc(NCC(=O)O)cc2cc1C1CCC(=O)NC1=O ; Cl ; Cc1nc2ccc(NCC(=O)N3CCN(CCn4c(C(=O)OC(C)(C)C)c(CCCOc5cccc6cc(F)ccc56)c5ccc(Cl)c(-c6c(C)nn(C)c6C)c54)CC3)cc2cc1C1CCC(=O)NC1=O ; Cc1nc2ccc(NCC(=O)N3CCN(CCn4c(C(=O)O)c(CCCOc5cccc6cc(F)ccc56)c5ccc(Cl)c(-c6c(C)nn(C)c6C)c54)CC3)cc2cc1C1CCC(=O)NC1=O]

### Step A

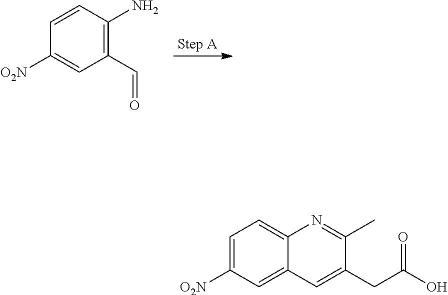 [SMILES: Nc1ccc([N+](=O)[O-])cc1C=O ; Cc1nc2ccc([N+](=O)[O-])cc2cc1CC(=O)O]

To a stirred solution of 2-amino-5-nitrobenzaldehyde (2.0 g, 12.0 mmol) in MeOH (32 mL) was added 4-oxopentanoic acid (1.2 mL, 12.0 mmol) followed by 2(N) aqueous sodium hydroxide solution (8 mL) drop-wise at RT. Then the resultant reaction mixture was allowed to reflux for 18 h. The crude was concentrated under reduced pressure. Then it was neutralized with acetic acid and the obtained precipitate was filtered, washed with ether and pentane to get 2-(2-methyl-6-nitroquinolin-3-yl)acetic acid (2.0 g, 8.1 mmol, 67%) as brown solid.

LCMS (ESI+): m/z 247.2 [M+H]⁺

### Step B

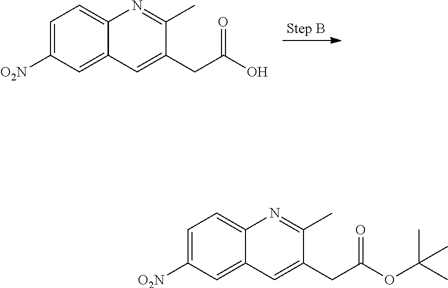 [SMILES: Cc1nc2ccc([N+](=O)[O-])cc2cc1CC(=O)O ; Cc1nc2ccc([N+](=O)[O-])cc2cc1CC(=O)OC(C)(C)C]

To a cooled solution of DCC (1.68 g, 8.13 mmol) in DCM (20 mL) was added DMAP (646 mg, 5.28 mmol) at 0° C. followed by 2-(2-methyl-6-nitroquinolin-3-yl)acetic acid (1.0 g, 4.05 mmol) and the resulting mixture was stirred at 0° C. for 5 min. Then tert-butanol (0.60 mL, 20.3 mmol) was added and the resulting reaction mixture was allowed to stir at room temperature for 16 h under nitrogen. After complete consumption of the starting material, the solvent was evaporated under reduced pressure to get the crude compound. It was then diluted with EtOAc and washed successively with water and brine. Organic layer was dried over anhydrous Na₂SO₄ and evaporated under reduced pressure to get the crude compound, which was then purified by column chromatography (SiO₂, 40% EtOAc in hexane) to obtain tert-butyl 2-(2-methyl-6-nitroquinolin-3-yl)acetate (500 mg, 1.65 mmol, 40.7%) as white solid.

LCMS (ESI+): m/z 302.8 [M+H]⁺

### Step C

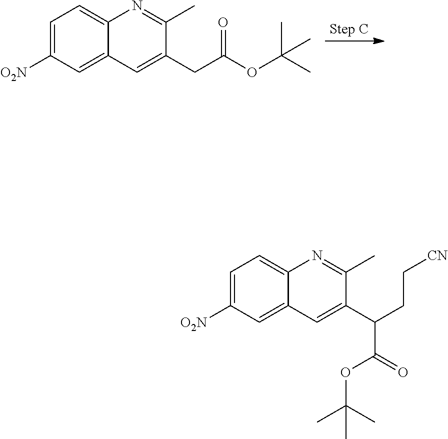 [SMILES: Cc1nc2ccc([N+](=O)[O-])cc2cc1CC(=O)OC(C)(C)C ; Cc1nc2ccc([N+](=O)[O-])cc2cc1C(CCC#N)C(=O)OC(C)(C)C]

To a stirred solution of tert-butyl 2-(2-methyl-6-nitroquinolin-3-yl)acetate (290 mg, 0.96 mmol) in DMF (250 mL) were added K₂CO₃ (132.7 mg, 0.96 mmol) followed by TEBAC (218.7 g, 0.96 mmol) under nitrogen atmosphere at 0° C. Then acrylonitrile (0.126 mL, 1.92 mmol) added into reaction mixture at RT. Then the resultant reaction mixture was allowed to stir at room temperature for 4 h under nitrogen. After complete consumption of the starting material, the reaction mixture was diluted with EtOAc, washed successively with cold water and brine, the organic layer was dried over anhydrous Na₂SO₄ and evaporated under reduced pressure. Crude compound was purified with column chromatography (SiO₂, 30% EtOAc in hexane) to get tert-butyl 4-cyano-2-(2-methyl-6-nitroquinolin-3-yl)butanoate (150 mg, 0.422 mmol, 44%) as brownish liquid.

LCMS (ESI+): m/z 356.2 [M+H]⁺

### Step D

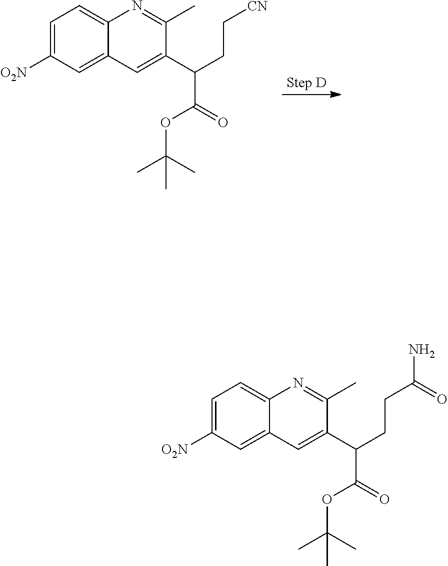 [SMILES: Cc1nc2ccc([N+](=O)[O-])cc2cc1C(CCC#N)C(=O)OC(C)(C)C ; Cc1nc2ccc([N+](=O)[O-])cc2cc1C(CCC(N)=O)C(=O)OC(C)(C)C]

To a stirred solution of tert-butyl 4-cyano-2-(2-methyl-6-nitroquinolin-3-yl)butanoate (150 mg, 0.422 mmol) in DMSO (5.0 mL) were added H₂O₂ (0.066 mL, 2.15 mmol) followed by K₂CO₃ (8.18 mg, 0.06 mmol) under nitrogen atmosphere at 0° C. Then the resultant reaction mixture was allowed stir at room temperature for 48 h under nitrogen. After complete consumption of the starting material, the reaction mixture was diluted with EtOAc, washed successively with cold water and brine. The organic layer was dried over Na₂SO₄ and evaporated under reduced pressure to get the crude compound, which was then purified by column chromatography (SiO₂, 80% EtOAc in hexane) to get tert-butyl 4-carbamoyl-2-(2-methyl-6-nitroquinolin-3-yl)butanoate (44 mg, 0.12 mmol, 28%) as yellow solid.

LCMS (ESI+): m/z 374.3 [M+H]⁺

### Step E

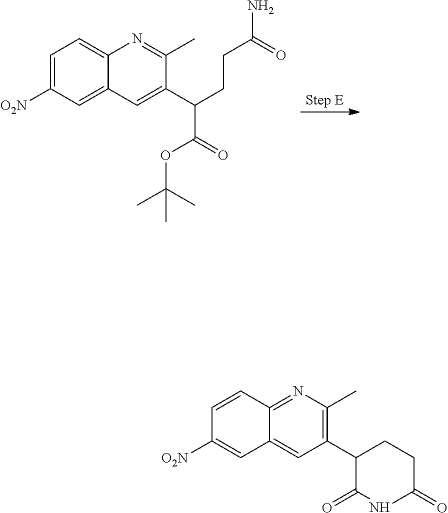 [SMILES: Cc1nc2ccc([N+](=O)[O-])cc2cc1C(CCC(N)=O)C(=O)OC(C)(C)C ; Cc1nc2ccc([N+](=O)[O-])cc2cc1C1CCC(=O)NC1=O]

tert-Butyl 4-carbamoyl-2-(2-methyl-6-nitroquinolin-3-yl)butanoate (100.0 mg, 0.268 mmol) and p-toluenesulfonic acid Monohydrate (509.4 mg, 2.678 mmol) were dissolved in MeCN (6.7 mL). To the solution was added Et₃N (0.372 mL, 2.678 mmol). The reaction mixture was stirred for next 3 days at 80° C. After complete consumption of the starting material, the solvent was evaporated and the crude was purified by reverse phase flash chromatography (C18, H₂O:MeCN+0.1% FA) to obtain 3-(2-methyl-6-nitroquinolin-3-yl)piperidine-2,6-dione (62.0 mg, 0.201 mmol, 75%) as a yellow solid.

LCMS (ESI+): m/z 300.3 [M+H]⁺

### Step F

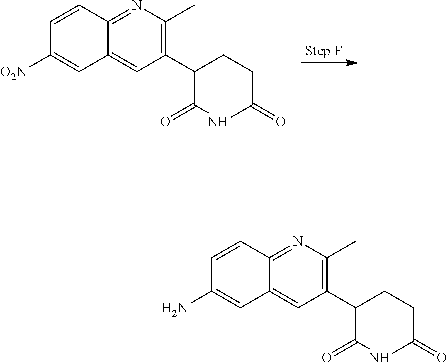 [SMILES: Cc1nc2ccc([N+](=O)[O-])cc2cc1C1CCC(=O)NC1=O ; Cc1nc2ccc(N)cc2cc1C1CCC(=O)NC1=O]

To a solution of 3-(2-methyl-6-nitroquinolin-3-yl)piperidine-2,6-dione (18.0 mg, 0.060 mmol) in EtOH (2.0 mL) was added Pd/C (10 mg) and the reaction mixture was degassed. Hydrogen was bubbled then through reaction mixture for 1 day at RT. The reaction mixture was filtered and concentrated under reduced pressure to obtain 3-(6-amino-2-methylquinolin-3-yl)piperidine-2,6-dione (11.0 mg, 0.041 mmol, 68%) as an yellow film.

LCMS (ESI+): m/z 270.1 [M+H]⁺

### Step G

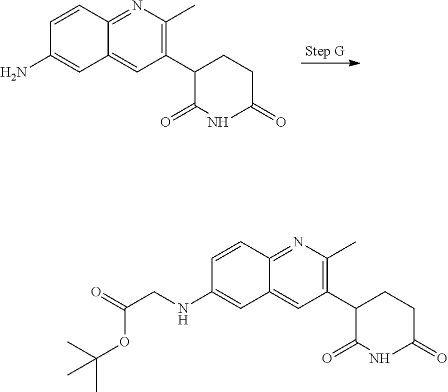 [SMILES: Cc1nc2ccc(N)cc2cc1C1CCC(=O)NC1=O ; Cc1nc2ccc(NCC(=O)OC(C)(C)C)cc2cc1C1CCC(=O)NC1=O]

3-(6-Amino-2-methylquinolin-3-yl)piperidine-2,6-dione (6.0 mg, 0.022 mmol) was dissolved in DMF (1.0 mL). KI (3.7 mg, 0.022 mmol) and KHCO₃ (6.7 mg, 0.067 mmol) were added to the solution, followed by addition of tert-butyl bromoacetate (0.003 mL, 0.022 mmol) in DMF (1.0 mL). Reaction was stirred at room temperature for 18 h. The crude was purified by reverse phase flash chromatography (C18, H₂O:MeCN+0.1% FA) to obtain tert-butyl 2-{[3-(2,6-dioxopiperidin-3-yl)-2-methylquinolin-6-yl]amino}acetate (5.0 mg, 0.013 mmol, 58%) as a yellow solid.

LCMS (ESI+): m/z 384.1 [M+H]⁺

### Step H

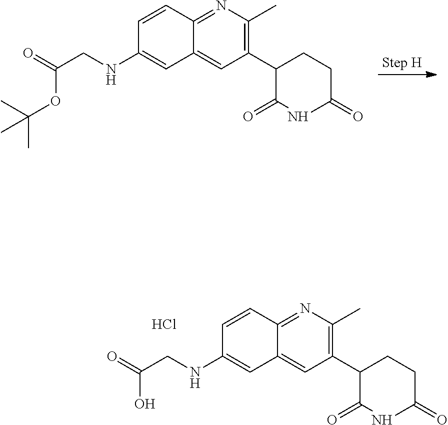 [SMILES: Cc1nc2ccc(NCC(=O)OC(C)(C)C)cc2cc1C1CCC(=O)NC1=O ; Cc1nc2ccc(NCC(=O)O)cc2cc1C1CCC(=O)NC1=O ; Cl]

The tert-butyl (3-(2,6-dioxopiperidin-3-yl)-2-methylquinolin-6-yl)glycinate (5.0 mg, 0.013 mmol) was solubilized in dioxane (2.0 mL) and the aqueous 36% HCl solution (2.0 mL) was added. The mixture was stirred for 18 h at RT. The volatiles were removed under reduced pressure and the residue was vacuum-dried. The crude (3-(2,6-dioxopiperidin-3-yl)-2-methylquinolin-6-yl)glycine hydrochloride (4.0 mg) was used directly for the next step.

LCMS (ESI+): m/z 328.2 [M+H]⁺

### Step I

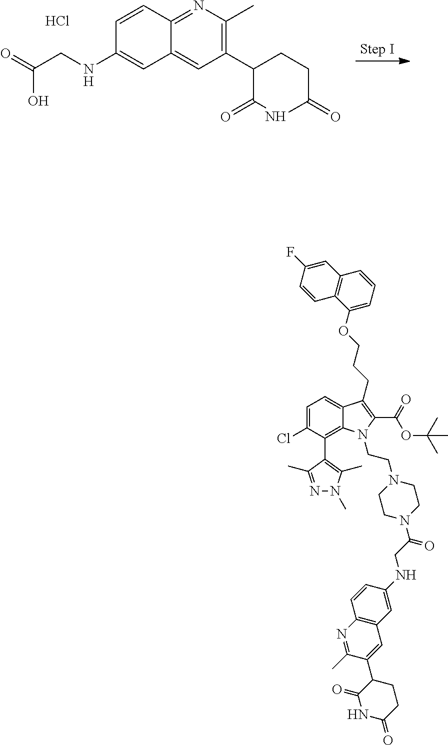

To a solution of 2-{[3-(2,6-dioxopiperidin-3-yl)-2-methylquinolin-6-yl]amino}acetic acid hydrochloride (5.8 mg, crude) and tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (10.0 mg, 0.015 mmol) in dry DMF (2.0 mL) were added HATU (11.3 mg, 0.030 mmol) and DIPEA (0.008 mL, 0.044 mmol). The reaction was stirred at room temperature for 15 min. The crude was diluted in DCM and washed with brine. The organic layer was dried over MgSO₄, filtered and concentrated in vacuo to obtain tert-butyl 6-chloro-1-{2-[4-(2-{[3-(2,6-dioxopiperidin-3-yl)-2-methylquinolin-6-yl]amino}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (26.2 mg, crude) as an yellow oil, which was used to next step without further purification.

LCMS (ESI+): m/z 983.7 [M+H]⁺

### Step J

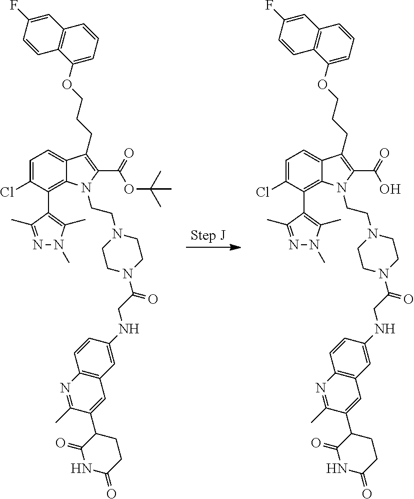

To a solution of tert-butyl 6-chloro-1-{2-[4-(2-{[3-(2,6-dioxopiperidin-3-yl)-2-methylquinolin-6-yl]amino}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (26.2 mg, crude) in DCM (1.0 mL) was added TFA (1.0 mL). The reaction mixture was stirred for 18 h at RT. The crude was concentrated in vacuo and purified by preparative HPLC (C18, H₂O:MeCN+0.1% FA) to obtain 6-chloro-1-{2-[4-(2-{[3-(2,6-dioxopiperidin-3-yl)-2-methylquinolin-6-yl]amino}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (3.8 mg, 0.004 mmol, 26% over 2 steps) as an yellow solid.

LCMS (ESI+): m/z 927.2 [M+H]⁺

¹H NMR (500 MHz, DMSO) δ 10.56 (s, 1H), 8.24 (dd, J=9.2, 5.9 Hz, 1H), 7.70-7.67 (m, 2H), 7.62 (d, J=9.0 Hz, 1H), 7.57 (dd, J=10.4, 2.6 Hz, 1H), 7.43-7.41 (m, 2H), 7.35-7.30 (m, 1H), 7.24 (dd, J=9.0, 2.6 Hz, 1H), 7.19 (d, J=8.5 Hz, 1H), 6.87 (dd, J=5.3, 3.4 Hz, 1H), 6.71 (d, J=2.6 Hz, 1H), 5.75-5.70 (m, 1H), 4.33-4.13 (m, 5H), 3.94 (d, J=4.6 Hz, 2H), 3.76 (s, 3H), 3.43-3.39 (m, 4H), 3.29-3.24 (m, 2H), 2.78 (ddd, J=17.3, 12.0, 5.3 Hz, 1H), 2.63-2.57 (m, 1H), 2.56 (s, 3H), 2.34-2.21 (m, 3H), 2.19-2.04 (m, 7H), 2.01 (d, J=1.7 Hz, 3H), 1.89 (s, 3H).

### Example 133: 6-Chloro-1-{2-[4-(2-{[3-(2,6-dioxopiperidin-3-yl)-2-methylquinolin-5-yl]amino}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (287)

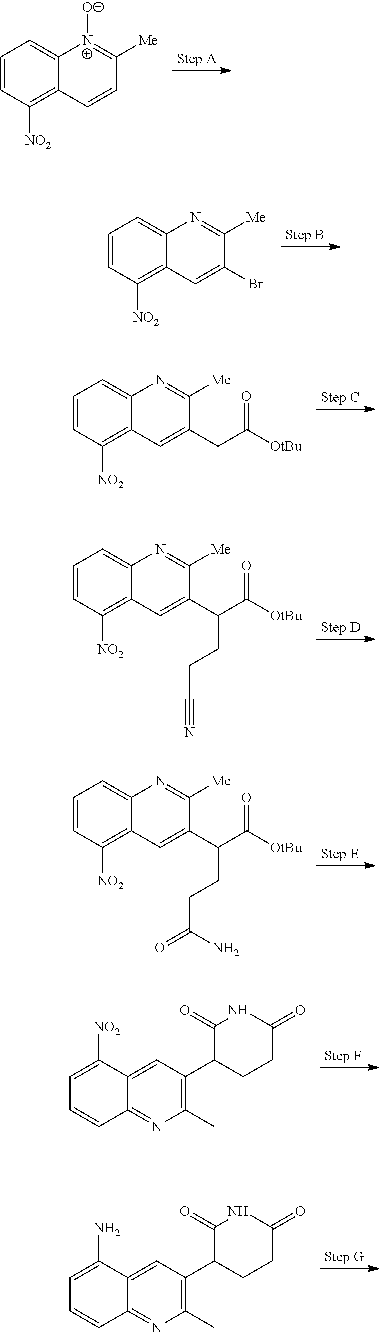

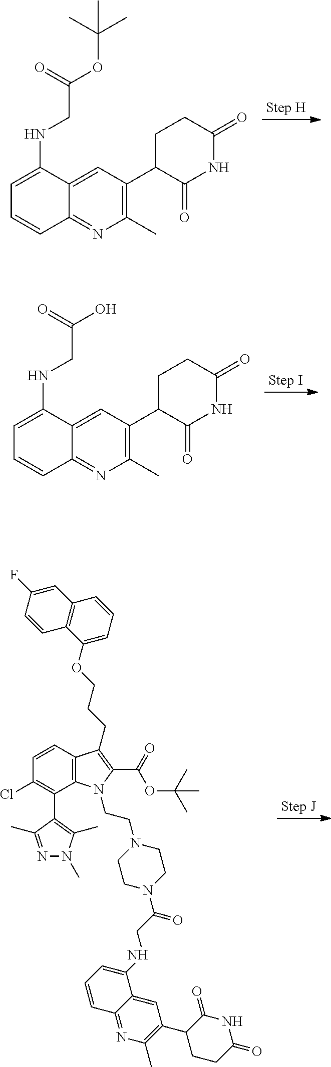 [SMILES: Cc1nc2cccc(NCC(=O)OC(C)(C)C)c2cc1C1CCC(=O)NC1=O ; Cc1nc2cccc(NCC(=O)O)c2cc1C1CCC(=O)NC1=O ; Cc1nc2cccc(NCC(=O)N3CCN(CCn4c(C(=O)OC(C)(C)C)c(CCCOc5cccc6cc(F)ccc56)c5ccc(Cl)c(-c6c(C)nn(C)c6C)c54)CC3)c2cc1C1CCC(=O)NC1=O]

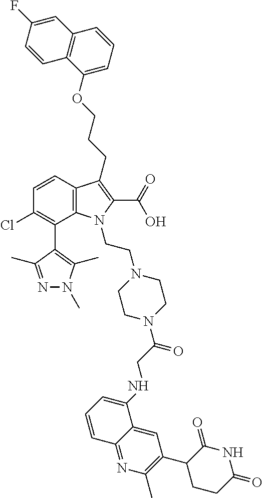 [SMILES: Cc1nc2cccc(NCC(=O)N3CCN(CCn4c(C(=O)O)c(CCCOc5cccc6cc(F)ccc56)c5ccc(Cl)c(-c6c(C)nn(C)c6C)c54)CC3)c2cc1C1CCC(=O)NC1=O]

### Step A

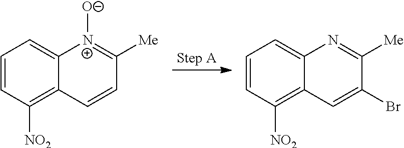 [SMILES: Cc1ccc2c([N+](=O)[O-])cccc2[n+]1[O-] ; Cc1nc2cccc([N+](=O)[O-])c2cc1Br]

To an ice cold solution of 2-methyl-5-nitroquinoline 1-oxide (500.0 mg, 2.44 mmol) in DCM (5 mL) was added POBr₃ (1.4 g, 4.9 mmol) in DCM (5 mL) portionwise at 0° C. Then the resulting reaction mixture was allowed to stir at room temperature for 48 h. After consumption of the starting material, ice water was added to the reaction mixture and then it was allowed to stir at room temperature for 1 h. Then it was neutralized with 10% aqueous NH₃ solution and extracted with DCM. Organic layer was washed with brine and dried over Na₂SO₄, filtered and concentrated to get crude which was purified with column chromatography (SiO₂, 10% EtOAc in hexane) to afford 2-methyl-3-bromo-5-nitroquinoline 1-oxide (100 mg, 0.37 mmol, 14.4%).

### Step B

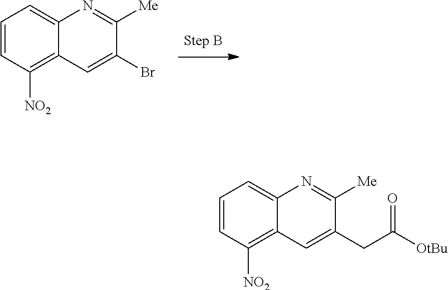 [SMILES: Cc1nc2cccc([N+](=O)[O-])c2cc1Br ; Cc1nc2cccc([N+](=O)[O-])c2cc1CC(=O)OC(C)(C)C]

To a stirred solution of 2-methyl-3-bromo-5-nitroquinoline 1-oxide (600 mg, 2.24 mmol) in dioxane (8.0 mL) was added KOAc (441 mg, 4.49 mmol) followed by the addition of ((1-(tert-butoxy)vinyl)oxy)(tert-butyl)dimethylsilane (2.07 g, 8.98 mmol) under nitrogen atmosphere at room temperature in a sealed tube, then reaction mixture was degassed for 15 min under nitrogen. Then was added Pd[(O-Tol)₃P]Cl₂ (353.2 mg, 0.449 mmol), and again degassed for 10 min at RT. Reaction mixture was heated to 130° C. and stirred in that temperature for 48 h. The mixture was diluted with EtOAc and filtered through Celite® pad and concentrated under reduced pressure to get crude which was purified with column chromatography (SiO₂, 10% EtOAc in hexane) to get tert-butyl 2-(2-methyl-5-nitroquinolin-3-yl)acetate (400 mg, 1.32 mmol, 58.89%).

LCMS (ESI+): m/z 303.2 [M+H]⁺

### Step C

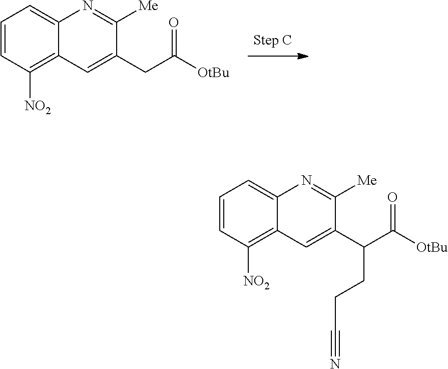 [SMILES: Cc1nc2cccc([N+](=O)[O-])c2cc1CC(=O)OC(C)(C)C ; Cc1nc2cccc([N+](=O)[O-])c2cc1C(CCC#N)C(=O)OC(C)(C)C]

To a stirred solution of tert-butyl 2-(2-methyl-5-nitroquinolin-3-yl)acetate (200 mg, 0.662 mmol) in DMF (10.0 mL) were added K₂CO₃ (150.6 mg, 0.662 mmol) followed by TEBAC (91.4 mg, 0.662 mmol) under nitrogen atmosphere at room temperature and stirred for 10 min. Then acrylonitrile was added (0.043 mL, 0.662 mmol) and the reaction was stirred at room temperature for 16 h. After completion, reaction mixture was partitioned between EtOAc and ice-cold water. Organic layer was dried over Na₂SO₄ and concentrated under reduced pressure to get crude which was purified with silica gel column chromatography (SiO₂, 55% EtOAc in hexane) to afford tert-butyl 4-cyano-2-(2-methyl-5-nitroquinolin-3-yl)butanoate (95 mg, 0.267 mmol, 40.41%).

LCMS (ESI+): m/z 356.2 [M+H]⁺

### Step D

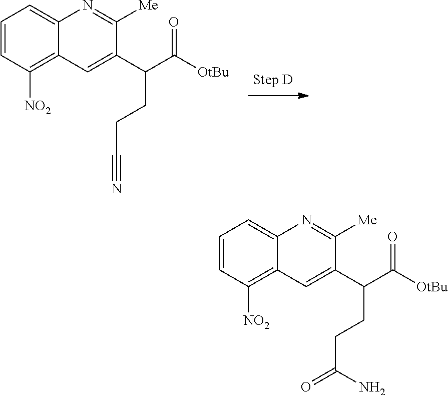 [SMILES: Cc1nc2cccc([N+](=O)[O-])c2cc1C(CCC#N)C(=O)OC(C)(C)C ; Cc1nc2cccc([N+](=O)[O-])c2cc1C(CCC(N)=O)C(=O)OC(C)(C)C]

To a solution of tert-butyl 4-cyano-2-(2-methyl-5-nitroquinolin-3-yl)butanoate (120.0 mg, 0.338 mmol) in DMSO (5.0 mL) was added H₂O₂ (0.052 mL, 1.688 mmol) followed by K₂CO₃ (6.533 mg, 0.047 mmol) under nitrogen atmosphere at 0° C. and then allowed to stir at room temperature for 16 h. After completion, reaction mixture was partitioned between EtOAc and added ice-cold water. Organic layer was dried over Na₂SO₄ and concentrated under reduced pressure to afford crude which was purified by SFC (60% CO2 in MeOH) to get tert-butyl 5-amino-2-(2-methyl-5-nitroquinolin-3-yl)-5-oxopentanoate (65 mg, 0.17 mmol, 51%) as yellow solid.

LCMS (ESI+): m/z 374.2 [M+H]⁺

### Step E

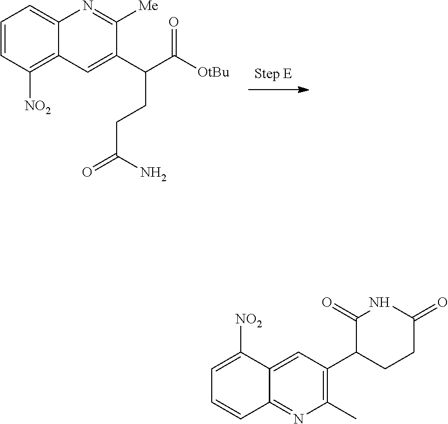 [SMILES: Cc1nc2cccc([N+](=O)[O-])c2cc1C(CCC(N)=O)C(=O)OC(C)(C)C ; Cc1nc2cccc([N+](=O)[O-])c2cc1C1CCC(=O)NC1=O]

In a vial was placed tert-butyl 5-amino-2-(2-methyl-5-nitroquinolin-3-yl)-5-oxopentanoate (250 mg, 0.67 mmol) and PTSA (1.273 g, 6.695 mmol), followed by MeCN (19.3 mL). The vial was sealed and heated to 80° C. for 18 h. The solvent was removed under reduced pressure, the residue was dissolved in DCM and washed with NaHCO₃aq, organic layer was concentrated and crude was purified using flash chromatography (SiO₂, DCM:MeOH gradient from 5 to 10% of MeOH) to give a corresponding 3-(2-methyl-5-nitroquinolin-3-yl)piperidine-2,6-dione (0.139 g, 0.464 mmol, 69.4%) as orange solid.

LCMS (ESI+): m/z 300.0 [M+H]⁺

### Step F

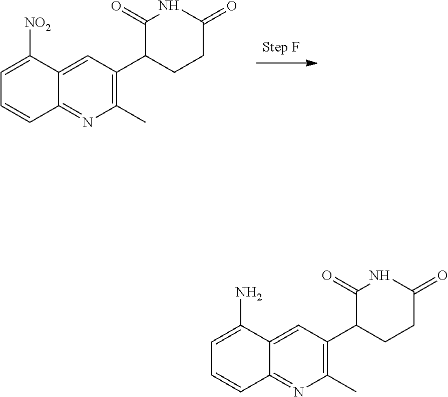 [SMILES: Cc1nc2cccc([N+](=O)[O-])c2cc1C1CCC(=O)NC1=O ; Cc1nc2cccc(N)c2cc1C1CCC(=O)NC1=O]

To the flask with 3-(2-methyl-5-nitroquinolin-3-yl)piperidine-2,6-dione (139.0 mg, 0.464 mmol) was added EtOH (14.8 mL) and the mixture was heated at 80° C. under condenser. After dissolution of the starting material the solution was purged with Argon, then Pd/C (14.0 mg) was added under Argon atmosphere. Then, Ar was displaced with H₂ and reaction was carried out under H₂ atmosphere for 18 h. Mixture was filtered and filtrate was evaporated and freeze-dried to give a 3-(5-amino-2-methylquinolin-3-yl)piperidine-2,6-dione (131 mg, 0.462 mmol, 99%) as yellow solid.

LCMS (ESI+): m/z 269.9 [M+H]⁺

### Step G

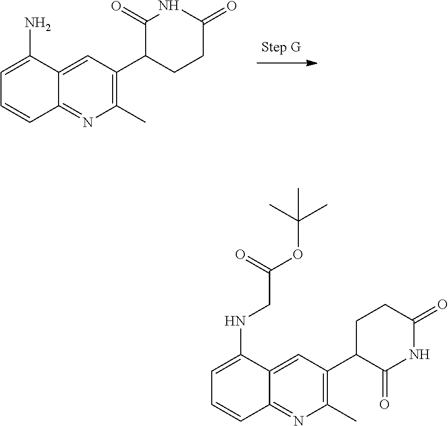 [SMILES: Cc1nc2cccc(N)c2cc1C1CCC(=O)NC1=O ; Cc1nc2cccc(NCC(=O)OC(C)(C)C)c2cc1C1CCC(=O)NC1=O]

In a vial 3-(5-amino-2-methylquinolin-3-yl)piperidine-2,6-dione (50.0 mg, 0.186 mmol) was dissolved in DMF (509 μL) and KI (30.8 mg, 0.186 mmol) along with KHCO₃ (55.8 mg, 0.557 mmol) were added, followed by dropwise addition of tert-butyl bromoacetate (27 μL, 0.186 mmol) in DMF (509 μL). Reaction was stirred for 60° C. for 5 h. Reaction mixture was directly purified with reverse phase flash chromatography (H₂O:MeCN+0.1% FA) to give a corresponding tert-butyl 2-{[3-(2,6-dioxopiperidin-3-yl)-2-methylquinolin-5-yl]amino}acetate (30.0 mg, 0.078 mmol, 42.1%) as a yellow solid.

LCMS (ESI+): m/z 384.1 [M+H]⁺

### Step H

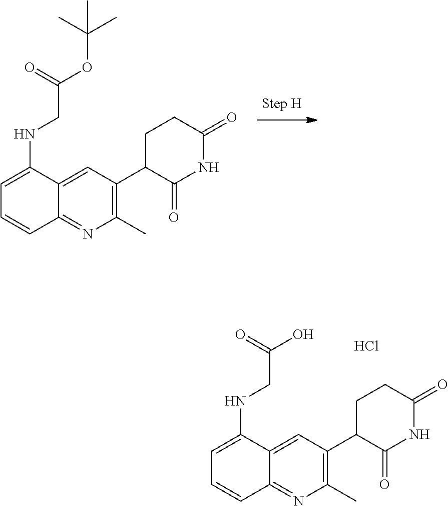 [SMILES: Cc1nc2cccc(NCC(=O)OC(C)(C)C)c2cc1C1CCC(=O)NC1=O ; Cc1nc2cccc(NCC(=O)O)c2cc1C1CCC(=O)NC1=O ; Cl]

tert-Butyl 2-{[3-(2,6-dioxopiperidin-3-yl)-2-methylquinolin-5-yl]amino}acetate (24.0 mg, 0.063 mmol) was dissolved in DCM (480 μL), and TFA (200 μL, 2.608 mmol) was added. Mixture was stirred at room temperature overnight. Solvents were removed under reduced pressure, and product was co-evaporated 3× with 1M HCl. 2-{[3-(2,6-Dioxopiperidin-3-yl)-2-methylquinolin-5-yl]amino}acetic acid hydrochloride (22.0 mg, 0.060 mmol, 96.6%,) was isolated as an orange solid.

LCMS (ESI+): m/z 328.0 [M+H]⁺

### Step I

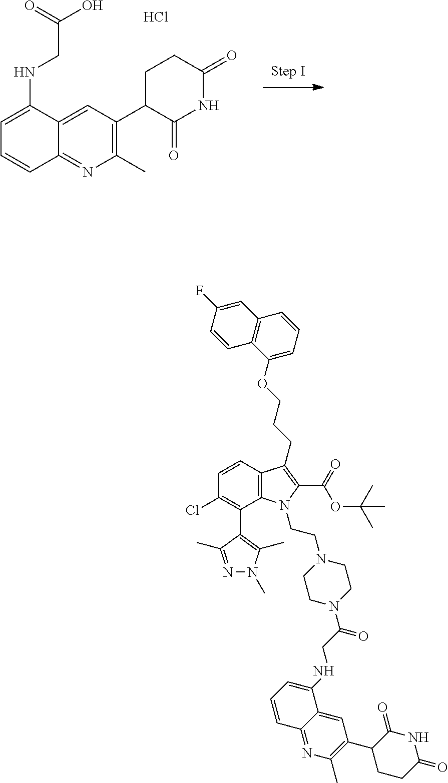 [SMILES: Cc1nc2cccc(NCC(=O)O)c2cc1C1CCC(=O)NC1=O ; Cl ; Cc1nc2cccc(NCC(=O)N3CCN(CCn4c(C(=O)OC(C)(C)C)c(CCCOc5cccc6cc(F)ccc56)c5ccc(Cl)c(-c6c(C)nn(C)c6C)c54)CC3)c2cc1C1CCC(=O)NC1=O]

2-{[3-(2,6-Dioxopiperidin-3-yl)-2-methylquinolin-5-yl]amino}acetic acid hydrochloride (19.4 mg, 0.053 mmol), tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (30.0 mg, 0.044 mmol) and HATU (22.0 mg, 0.058 mmol) were dissolved in dry DMF (1.1 mL) and to the mixture was added DIPEA (0.039 mL, 0.222 mmol). The reaction was stirred at room temperature for 2 h. The solvent was evaporated. Residues were dissolved in DCM and washed with brine. The organic layer was dried over Na₂SO₄, filtered and concentrated in vacuo. The crude tert-butyl 6-chloro-1-{2-[4-(2-{4-[(2,6-dioxopiperidin-3-yl)carbamoyl]-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (20.0 mg) was used without further purification in the next step.

LCMS (ESI+): m/z 983.95 [M+H]⁺

### Step J

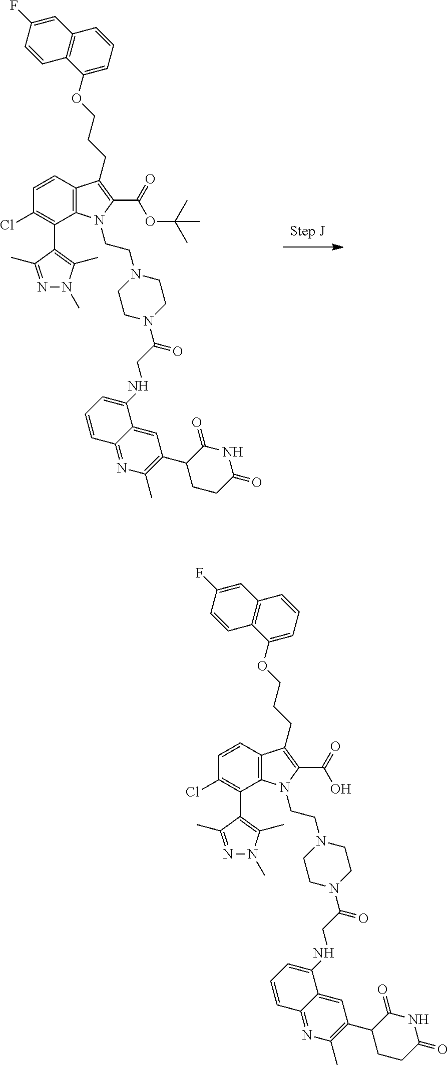 [SMILES: Cc1nc2cccc(NCC(=O)N3CCN(CCn4c(C(=O)OC(C)(C)C)c(CCCOc5cccc6cc(F)ccc56)c5ccc(Cl)c(-c6c(C)nn(C)c6C)c54)CC3)c2cc1C1CCC(=O)NC1=O ; Cc1nc2cccc(NCC(=O)N3CCN(CCn4c(C(=O)O)c(CCCOc5cccc6cc(F)ccc56)c5ccc(Cl)c(-c6c(C)nn(C)c6C)c54)CC3)c2cc1C1CCC(=O)NC1=O]

To the solution of crude tert-butyl 6-chloro-1-{2-[4-(2-{[3-(2,6-dioxopiperidin-3-yl)-2-methylquinolin-5-yl]amino}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (20.0 mg) in DCM (670 μL) was added TFA (670 μL, 8.749 mmol), and the mixture was stirred overnight at room temperature. Upon completion, solvents were removed under reduced pressure and crude was purified by preparative HPLC (C18, H₂O:MeCN+0.1% FA) to give a corresponding 6-chloro-1-{2-[4-(2-{[3-(2,6-dioxopiperidin-3-yl)-2-methylquinolin-5-yl]amino}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (12.1 mg, 0.013 mmol, 29.5% over 2 steps) as a white solid.

LCMS (ESI+): m/z 927.4 [M+H]⁺

¹H NMR (500 MHz, DMSO, 353 K) δ=10.57 (s, 1H), 8.24 (dd, J=9.2, 5.9, 1H), 8.19 (s, 1H), 7.71 (d, J=8.5, 1H), 7.58 (dd, J=10.4, 2.7, 1H), 7.45-7.38 (m, 3H), 7.32 (td, J=8.9, 2.6, 1H), 7.22 (d, J=8.5, 1H), 7.17 (d, J=8.3, 1H), 6.87 (dd, J=5.2, 3.4, 1H), 6.47 (d, J=7.7, 1H), 6.16-6.07 (m, 1H), 4.33-4.15 (m, 5H), 4.02 (d, J=4.9, 2H), 3.76 (s, 3H), 3.46-3.40 (m, 4H), 3.32-3.25 (m, 2H), 2.84 (ddd, J=17.1, 12.8, 5.4, 1H), 2.69-2.63 (m, 1H), 2.62 (s, 3H), 2.42 (qd, J=12.8, 4.3, 1H), 2.29-2.20 (m, 2H), 2.19-2.01 (m, 7H), 2.01 (d, J=1.4, 3H), 1.89 (s, 3H).

### Example 134: 6-Chloro-1-(2-(4-(2-((3-(2,6-dioxopiperidin-3-yl)-2-methylquinolin-7-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (288)

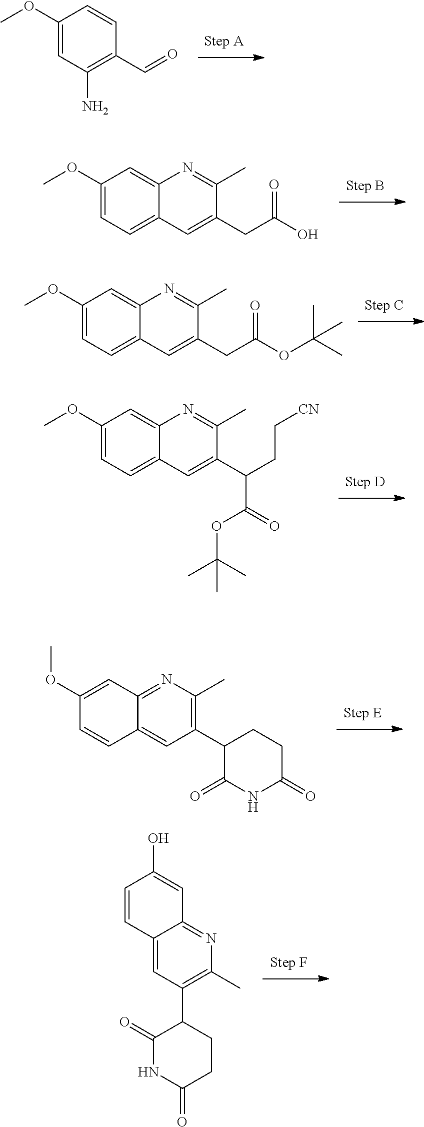

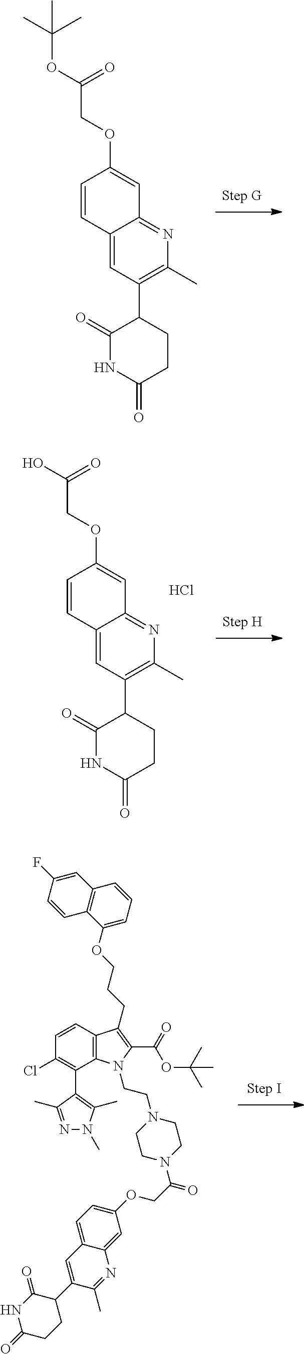 [SMILES: Cc1nc2cc(OCC(=O)OC(C)(C)C)ccc2cc1C1CCC(=O)NC1=O ; Cc1nc2cc(OCC(=O)O)ccc2cc1C1CCC(=O)NC1=O ; Cl ; Cc1nc2cc(OCC(=O)N3CCN(CCn4c(C(=O)OC(C)(C)C)c(CCCOc5cccc6cc(F)ccc56)c5ccc(Cl)c(-c6c(C)nn(C)c6C)c54)CC3)ccc2cc1C1CCC(=O)NC1=O]

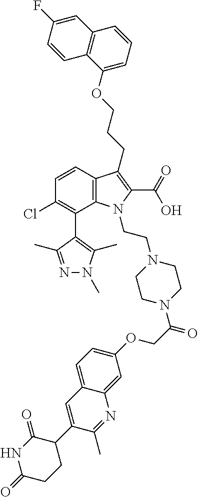 [SMILES: Cc1nc2cc(OCC(=O)N3CCN(CCn4c(C(=O)O)c(CCCOc5cccc6cc(F)ccc56)c5ccc(Cl)c(-c6c(C)nn(C)c6C)c54)CC3)ccc2cc1C1CCC(=O)NC1=O]

### Step A

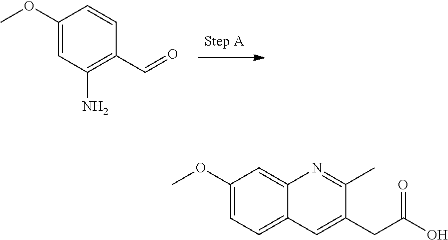 [SMILES: COc1ccc(C=O)c(N)c1 ; COc1ccc2cc(CC(=O)O)c(C)nc2c1]

To a stirred solution of 2-amino-4-methoxybenzaldehyde (5 g, 33.11 mmol) and 4-oxopentanoic acid (3.84 g, 33.11 mmol) in MeOH (60 mL) was added 2M NaOH Solution (20 mL) at 0° C. Then the resulting reaction mixture was allowed to stir at room temperature for 16 h. After consumption of the starting material, the reaction mixture was concentrated to get crude product, which was acidified with acetic acid (pH=6) and extracted with EtOAc. The obtained organic layers were evaporated under reduced pressure to get 2-(7-methoxy-2-methylquinolin-3-yl)acetic acid (3.6 g, 15.58 mmol, 47%) as an off-white solid.

LCMS (ESI+): m/z 232.0 [M+H]⁺

### Step B

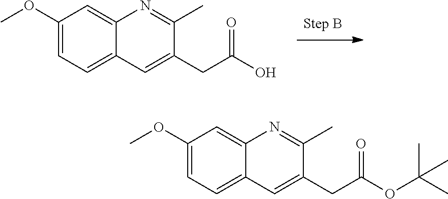 [SMILES: COc1ccc2cc(CC(=O)O)c(C)nc2c1 ; COc1ccc2cc(CC(=O)OC(C)(C)C)c(C)nc2c1]

To a cooled solution of DCC (786 mg, 3.81 mmol) in DCM (5 mL) was added DMAP (349.057 mg, 2.857 mmol) at 0° C. followed by 2-(7-methoxy-2-methylquinolin-3-yl)acetic acid (440.0 mg, 1.905 mmol) and the resulting mixture was stirred at 0° C. for 5 min. Then tert-butanol (0.9 mL, 0.905 mmol) was added and the resulting reaction mixture was allowed to stir at room temperature for 16 h under nitrogen. After complete consumption of the starting material the volatiles were evaporated under reduced pressure. Crude product was diluted with EtOAC, washed successively with water and brine, organic layer was dried over Na₂SO₄ and evaporated under reduced pressure. Crude product was purified with column chromatography (SiO₂, 50-60% EtOAc in hexane) to get tert-butyl 2-(7-methoxy-2-methylquinolin-3-yl)acetate (400 mg, 1.39 mmol, 73.08%) as an off white solid.

LCMS (ESI+): m/z 231.6 [M+H]⁺

### Step C

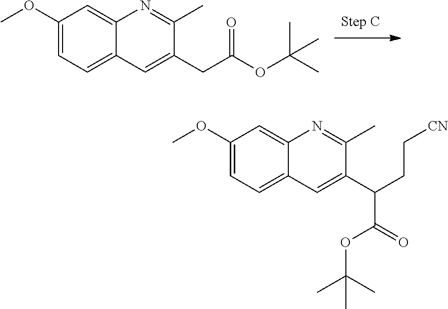

To a stirred solution of tert-butyl 2-(7-methoxy-2-methylquinolin-3-yl)acetate (250 mg, 0.871 mmol) in DMF (3 mL) was added TEBAC (198.3 mg, 0.871 mmol) and K₂CO₃ (120.3 mg, 0.871 mmol) at 0° C., then resultant reaction mixture was stirred at same temperature for 15 min. After that acrylonitrile (0.085 mL, 1.306 mmol) was added at 0° C. Resultant reaction mixture was stirred at room temperature for 16 h. Progress of the reaction was monitored by TLC. The reaction mixture was quenched with water and extracted with EtOAc, then organic layer was washed with chilled brine solution. The organic layer was evaporated under reduced pressure to get the crude compound, which was then purified by column chromatography (SiO₂, 50% EtOAc in hexane) to get tert-butyl 4-cyano-2-(7-methoxy-2-methylquinolin-3-yl)butanoate (100 mg, 0.293 mmol, 33.74%) as a pale yellow solid.

LCMS (ESI+): m/z 340.8 [M+H]⁺

### Step D

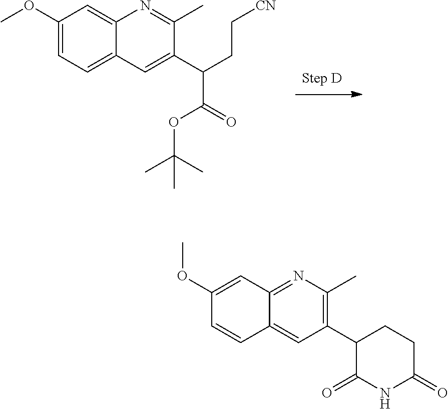 [SMILES: COc1ccc2cc(C(CCC#N)C(=O)OC(C)(C)C)c(C)nc2c1 ; COc1ccc2cc(C3CCC(=O)NC3=O)c(C)nc2c1]

To a cooled solution of tert-butyl 4-cyano-2-(7-methoxy-2-methylquinolin-3-yl)butanoate (100.0 mg, 0.293 mmol) in MeOH (5 mL) were added H₂O₂ (0.5 mL, 0.293 mmol) and K₂CO₃ (40.521 mg, 0.293 mmol) at 0° C. The resulting mixture was stirred at room temperature for 16 h. After complete consumption of the starting material reaction the mixture was distilled to get crude, which was dissolved in MeCN and then was added PTSA (100.976 mg, 0.586 mmol) at 0° C. Resulting reaction mixture was refluxed for 16 h. After complete consumption of the starting material volatiles were evaporated under reduced pressure to get crude which was diluted with EtOAc and washed successively with NaHCO₃ solution and brine. Organic layer was dried over Na₂SO₄ and evaporated under reduced pressure. Crude was then purified by column chromatography (SiO₂, 50-60% EtOAc in hexane) to get 3-(7-methoxy-2-methylquinolin-3-yl) piperidine-2,6-dione (10 mg, 0.025 mmol 30%) as off white solid.

LCMS (ESI+): m/z 285.43 [M+H]⁺

### Step E

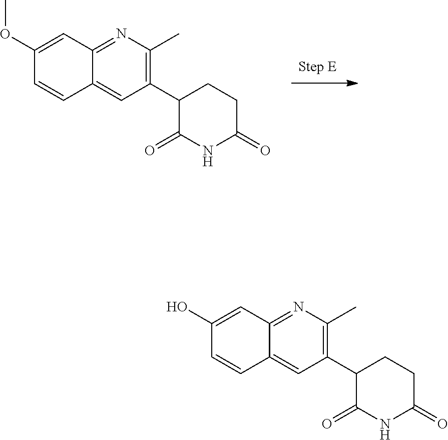 [SMILES: COc1ccc2cc(C3CCC(=O)NC3=O)c(C)nc2c1 ; Cc1nc2cc(O)ccc2cc1C1CCC(=O)NC1=O]

A mixture of 3-(7-methoxy-2-methylquinolin-3-yl) piperidine-2,6-dione (100 mg 0.246 mmol), thiophenol (0.25 mL, 2.46 mmol) and K₂CO₃ (17 mg, 0.123 mmol) in NMP (3 mL) was stirred under microwave irradiation at 190° C. for 30 minutes. After complete consumption of the starting material, the reaction mixture was filtered and the filtrate was purified by preparative HPLC (ammonium acetate buffer:MeCN) to get 3-(7-hydroxy-2-methylquinolin-3-yl)piperidine-2,6-dione (40 mg, 0.148 mmol, 60%) as off white solid.

LCMS (ESI+): m/z 271.1 [M+H]⁺

### Step F

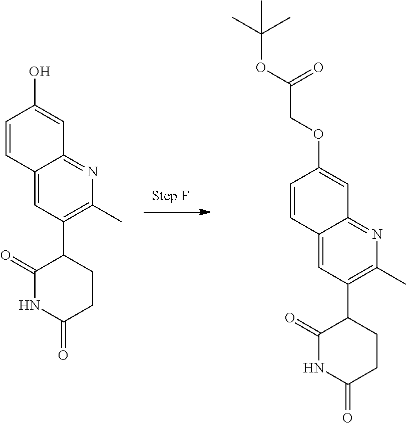 [SMILES: Cc1nc2cc(O)ccc2cc1C1CCC(=O)NC1=O ; Cc1nc2cc(OCC(=O)OC(C)(C)C)ccc2cc1C1CCC(=O)NC1=O]

In a vial 3-(7-hydroxy-2-methylquinolin-3-yl)piperidine-2,6-dione (50.0 mg, 0.185 mmol) was dissolved in DMF (507 μL) and KI (30.7 mg, 0.185 mmol) along with KHCO₃ (55.6 mg, 0.555 mmol) were added, followed by dropwise addition of tert-butyl bromoacetate (27 μL, 0.185 mmol) in DMF (507 μL). Reaction was stirred for 60° C. for 5 h. Reaction mixture was purified directly with reverse phase flash chromatography (H₂O:MeCN+0.1% FA) to give a corresponding tert-butyl 2-{[3-(2,6-dioxopiperidin-3-yl)-2-methylquinolin-7-yl]oxy}acetate (36.0 mg, 0.094 mmol, 50.6%,) as white solid.

LCMS (ESI+): m/z 385.11 [M+H]⁺

### Step G

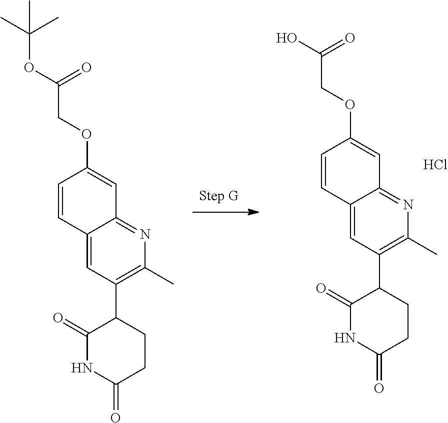 [SMILES: Cc1nc2cc(OCC(=O)OC(C)(C)C)ccc2cc1C1CCC(=O)NC1=O ; Cc1nc2cc(OCC(=O)O)ccc2cc1C1CCC(=O)NC1=O ; Cl]

tert-Butyl 2-{[3-(2,6-dioxopiperidin-3-yl)-2-methylquinolin-7-yl]oxy}acetate (36.0 mg, 0.094 mmol) was dissolved in DCM (718 μL), and TFA (200 μL, 2.608 mmol) was added. Mixture was stirred in room temperature for overnight. Solvents were removed under reduced pressure, and product was co-evaporated 3× with 1M HCl. 2-{[3-(2,6-dioxopiperidin-3-yl)-2-methylquinolin-7-yl]oxy}acetic acid hydrochloride (27.4 mg, 0.075 mmol, 79.8%) was isolated as green solid.

LCMS (ESI+): m/z 329.05 [M+H]⁺

### Step H

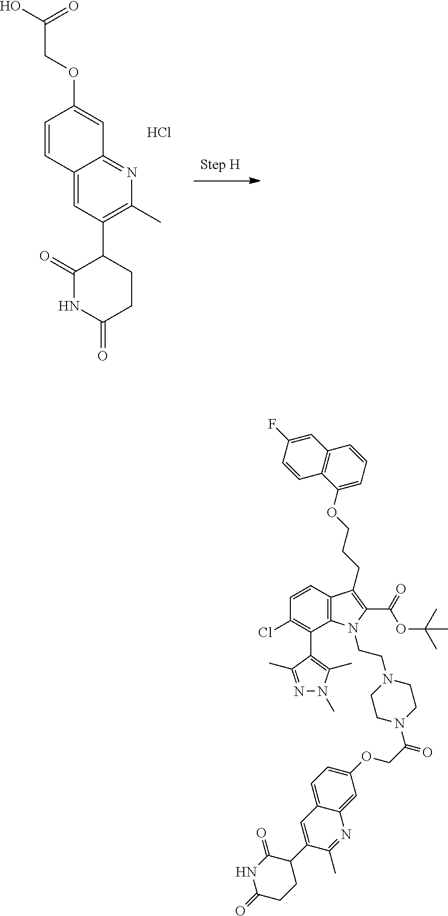 [SMILES: Cc1nc2cc(OCC(=O)O)ccc2cc1C1CCC(=O)NC1=O ; Cl ; Cc1nc2cc(OCC(=O)N3CCN(CCn4c(C(=O)OC(C)(C)C)c(CCCOc5cccc6cc(F)ccc56)c5ccc(Cl)c(-c6c(C)nn(C)c6C)c54)CC3)ccc2cc1C1CCC(=O)NC1=O]

2-{[3-(2,6-Dioxopiperidin-3-yl)-2-methylquinolin-7-yl]oxy}acetic acid hydrochloride (14.9 mg, 0.041 mmol), tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (25.0 mg, 0.037 mmol) and HATU (18.3 mg, 0.048 mmol) were dissolved in dry DMF (882 μL) and to the mixture was added DIPEA (32 μL, 0.185 mmol). The reaction was stirred at room temperature for 18 h. Crude mixture was purified by preparative HPLC (H₂O:MeCN+0.1% FA), followed by preparative TLC (SiO₂ 10% of MeOH in DCM). Desired product tert-butyl 6-chloro-1-{2-[4-(2-{4-[(2,6-dioxopiperidin-3-yl)carbamoyl]-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (13.9 mg, 0.014 mmol, 38.1%) was used directly to next step.

LCMS (ESI+): m/z 984.41 [M+H]⁺

### Step I

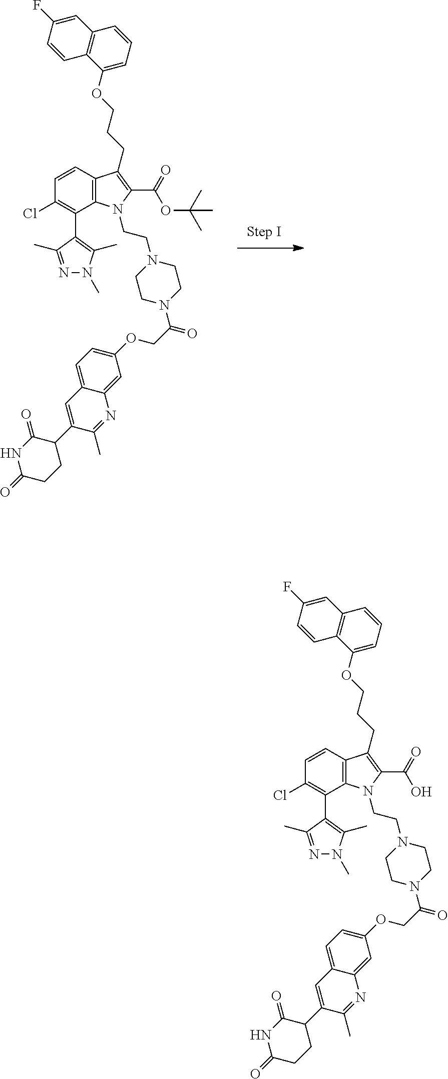 [SMILES: Cc1nc2cc(OCC(=O)N3CCN(CCn4c(C(=O)OC(C)(C)C)c(CCCOc5cccc6cc(F)ccc56)c5ccc(Cl)c(-c6c(C)nn(C)c6C)c54)CC3)ccc2cc1C1CCC(=O)NC1=O ; Cc1nc2cc(OCC(=O)N3CCN(CCn4c(C(=O)O)c(CCCOc5cccc6cc(F)ccc56)c5ccc(Cl)c(-c6c(C)nn(C)c6C)c54)CC3)ccc2cc1C1CCC(=O)NC1=O]

To solution of tert-butyl 6-chloro-1-{2-[4-(2-{[3-(2,6-dioxopiperidin-3-yl)-2-methylquinolin-7-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (13.0 mg, 0.013 mmol) in DCM (436 μL) was added TFA (436 μL, 5.687 mmol), and the mixture was stirred for overnight in room temperature. Upon completion, Solvents were removed under reduced pressure and crude was purified with preparative HPLC (H₂O:MeCN+0.1% FA) to give a corresponding 6-chloro-1-{2-[4-(2-{[3-(2,6-dioxopiperidin-3-yl)-2-methylquinolin-7-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (8.2 mg, 0.009 mmol, 66.5%) as a white solid.

LCMS (ESI+): m/z 928.2 [M+H]⁺

¹H NMR (500 MHz, DMSO, 353 K) δ=10.56 (s, 1H), 8.24 (dd, J=9.2, 5.9, 1H), 7.97 (s, 1H), 7.76 (d, J=8.9, 1H), 7.70 (d, J=8.5, 1H), 7.58 (dd, J=10.4, 2.6, 1H), 7.44-7.38 (m, 2H), 7.35-7.28 (m, 2H), 7.21 (d, J=8.5, 1H), 7.18 (dd, J=8.9, 2.6, 1H), 6.87 (dd, J=5.6, 3.0, 1H), 4.88 (s, 2H), 4.34-4.13 (m, 5H), 3.75 (s, 3H), 3.43-3.36 (m, 4H), 3.31-3.23 (m, 2H), 2.81 (ddd, J=17.6, 12.5, 5.4, 1H), 2.65-2.59 (m, 4H), 2.39-2.30 (m, 1H), 2.29-2.19 (m, 2H), 2.19-2.04 (m, 7H), 2.00 (s, 3H), 1.88 (s, 3H).

### Example 135. 6-Chloro-1-{2-[4-(2-{[3-(2,6-dioxopiperidin-3-yl)-2-methylquinolin-6-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (289)

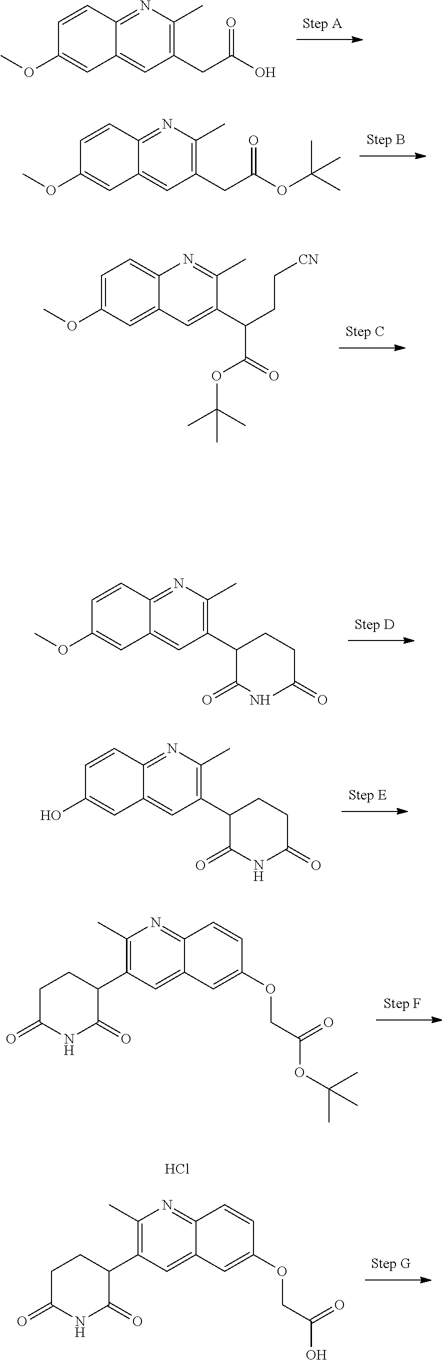

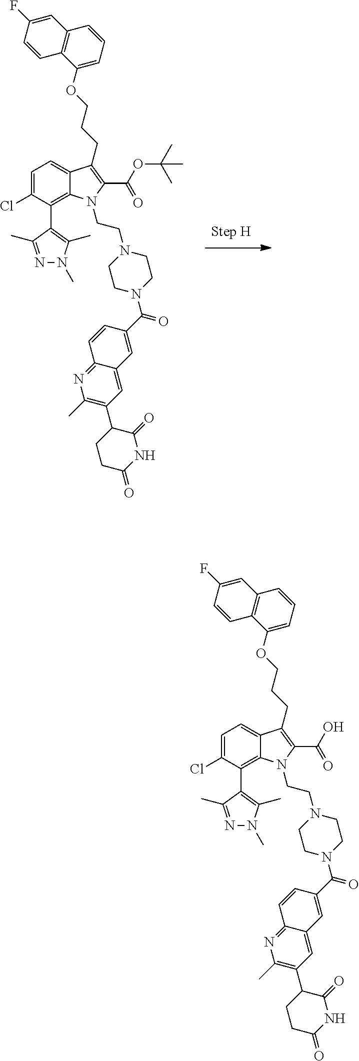 [SMILES: Cc1nc2ccc(C(=O)N3CCN(CCn4c(C(=O)OC(C)(C)C)c(CCCOc5cccc6cc(F)ccc56)c5ccc(Cl)c(-c6c(C)nn(C)c6C)c54)CC3)cc2cc1C1CCC(=O)NC1=O ; Cc1nc2ccc(C(=O)N3CCN(CCn4c(C(=O)O)c(CCCOc5cccc6cc(F)ccc56)c5ccc(Cl)c(-c6c(C)nn(C)c6C)c54)CC3)cc2cc1C1CCC(=O)NC1=O]

### Step A

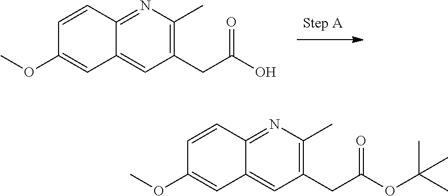 [SMILES: COc1ccc2nc(C)c(CC(=O)O)cc2c1 ; COc1ccc2nc(C)c(CC(=O)OC(C)(C)C)cc2c1]

To a cooled solution of DCC (3.5 g, 16.9 mmol) in DCM (24 mL) was added DMAP (1.34 g, 10.9 mmol) at 0° C. followed by 2-(6-methoxy-2-methylquinolin-3-yl)acetic acid (1.95 g, 8.43 mmol) and the resulting mixture was stirred at 0° C. for 5 min. Then tert-butanol (12.1 mL, 126.6 mmol) was added and the resulting reaction mixture was allowed to stir at room temperature for 12 h under nitrogen. After complete consumption of the starting material (monitored by TLC and LCMS), the solvent was evaporated under reduced pressure to get the crude compound. It was then diluted with EtOAc and washed successively with water and brine. Organic layer was dried over Na₂SO₄ and evaporated under reduced pressure to get the crude compound, which was then purified with column chromatography (SiO₂, 20-30% EtOAc in hexane) to get tert-butyl 2-(6-methoxy-2-methylquinolin-3-yl)acetate (1 g, 3.48 mmol, 41%) as off white solid.

LCMS (ESI+): m/z 287.8 [M+H]⁺

### Step B

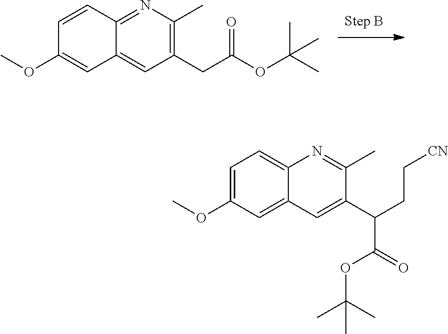

To a stirred solution of tert-butyl 2-(6-methoxy-2-methylquinolin-3-yl)acetate (1 g, 3.48 mmol) in DMF (15 mL) was added K₂CO₃ (0.48 g, 3.48 mmol) followed by TEBAC (0.792 g, 3.48 mmol) under nitrogen atmosphere at 0° C. Then acrylonitrile (0.185 mL, 3.82 mmol) was added into reaction mixture and the resulting reaction mixture was allowed to stir at room temperature for 6 h under nitrogen. After complete consumption of the starting material (monitored by TLC and LCMS), the reaction mixture was diluted with EtOAc, and washed successively with cold water and brine The organic layer was dried over Na₂SO₄ and evaporated under reduced pressure to get the crude compound, which was then purified with column chromatography (SiO₂, 30-40% EtOAc in hexane) to get tert-butyl 4-cyano-2-(6-methoxy-2-methylquinolin-3-yl)butanoate (0.75 g, 2.2 mmol, 63%) as light yellow sticky solid.

LCMS (ESI+): m/z 341.0 [M+H]⁺

### Step C

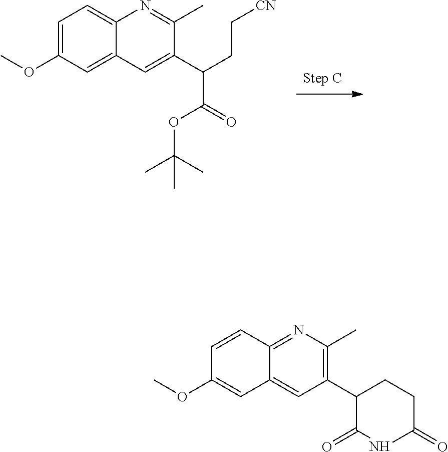 [SMILES: COc1ccc2nc(C)c(C(CCC#N)C(=O)OC(C)(C)C)cc2c1 ; COc1ccc2nc(C)c(C3CCC(=O)NC3=O)cc2c1]

To a stirred solution of tert-butyl 4-cyano-2-(6-methoxy-2-methylquinolin-3-yl)butanoate (0.6 g, 1.76 mmol) in MeOH (8 mL) was added H₂O₂ (0.3 mL, 8.8 mmol) followed by K₂CO₃ (242 mg, 1.75 mmol) under nitrogen atmosphere at 0° C. Then the resulting reaction mixture was allowed to stir at room temperature for 24 h under nitrogen. After complete consumption of the starting material (monitored by TLC and LCMS), the volatiles were evaporated under reduced pressure to get the crude compound which was suspended in MeCN (5 mL) and PTSA (0.42 g, 2.24 mmol) was added to it. The resulting reaction mixture was refluxed for 16 h. After complete consumption of starting material (monitored by LCMS), the acid was neutralized by Et₃N and evaporated to get the crude compound, which was then purified with column chromatography (SiO₂, 5-10% MeOH in DCM) to get 3-(6-methoxy-2-methylquinolin-3-yl)piperidine-2,6-dione (0.22 g, 0.774 mmol, 44%) as off white solid.

LCMS (ESI+): m/z 285.1 [M+H]⁺

### Step D

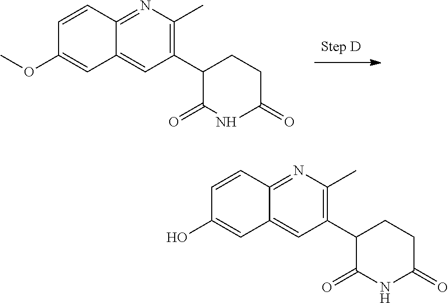 [SMILES: COc1ccc2nc(C)c(C3CCC(=O)NC3=O)cc2c1 ; Cc1nc2ccc(O)cc2cc1C1CCC(=O)NC1=O]

A mixture of 3-(6-methoxy-2-methylquinolin-3-yl)piperidine-2,6-dione (100 mg 0.35 mmol), thiophenol (0.38 mL, 3.5 mmol) and K₂CO₃ (24 mg, 0.17 mmol) in NMP (3 mL) was irradiated under microwave at 190° C. for 30 min. After complete consumption of the starting material (monitored by LCMS), the reaction mixture was filtered and the filtrate was purified by reverse phase preparative HPLC (C18, 20 mM ammonium acetate in H₂O:MeCN) to get 3-(6-hydroxy-2-methylquinolin-3-yl)piperidine-2,6-dione (30 mg, 0.111 mmol, 32%) as off white solid.

LCMS (ESI+): m/z 277.1 [M+H]⁺

### Step E

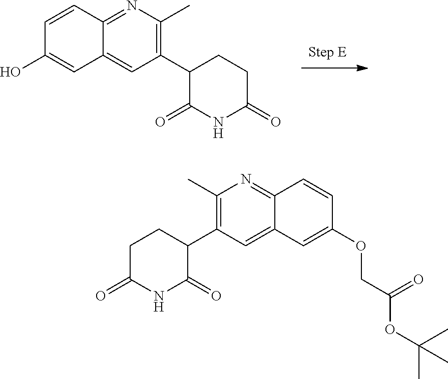 [SMILES: Cc1nc2ccc(O)cc2cc1C1CCC(=O)NC1=O ; Cc1nc2ccc(OCC(=O)OC(C)(C)C)cc2cc1C1CCC(=O)NC1=O]

3-(6-Hydroxy-2-methylquinolin-3-yl)piperidine-2,6-dione (35.0 mg, 0.129 mmol) was dissolved in DMF (2.0 mL) and KI (21.5 mg, 0.129 mmol) and KHCO₃ (38.9 mg, 0.388 mmol) were added, followed by addition of tert-butyl bromoacetate (0.023 mL, 0.155 mmol). Reaction was stirred for 60° C. for 6 h. The crude directly purified with reverse phase flash chromatography (C18, H₂O:MeCN+0.1% FA) to give a corresponding tert-butyl 2-{[3-(2,6-dioxopiperidin-3-yl)-2-methylquinolin-6-yl]oxy}acetate (23.5 mg, 0.061 mmol, 47%) as a white solid.

LCMS (ESI+): m/z 385.4 [M+H]⁺

### Step F

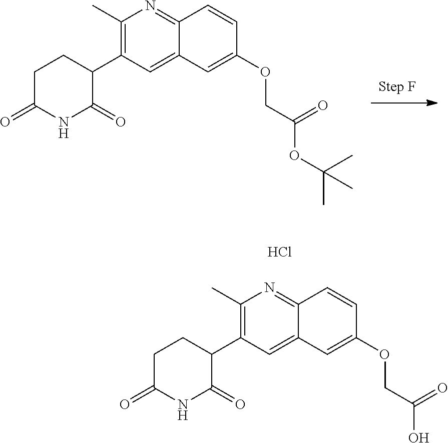 [SMILES: Cc1nc2ccc(OCC(=O)OC(C)(C)C)cc2cc1C1CCC(=O)NC1=O ; Cl ; Cc1nc2ccc(OCC(=O)O)cc2cc1C1CCC(=O)NC1=O]

To a solution of tert-butyl 2-{[3-(2,6-dioxopiperidin-3-yl)-2-methylquinolin-6-yl]oxy}acetate (23.5 mg, 0.061 mmol) in DCM (1.0 mL) was added TFA (1.0 mL, 13.059 mmol). The mixture was stirred for 5 h at RT. The crude was concentrated in vacuo and dissolved in H₂O. To the solution was added 36% HCl and it was evaporated. The product, 2-{[3-(2,6-dioxopiperidin-3-yl)-2-methylquinolin-6-yl]oxy}acetic acid hydrochloride (6.8 mg, 0.019 mmol, 31.1%) was isolated as brownish solid.

LCMS (ESI+): m/z 328.95 [M+H]⁺

### Step G

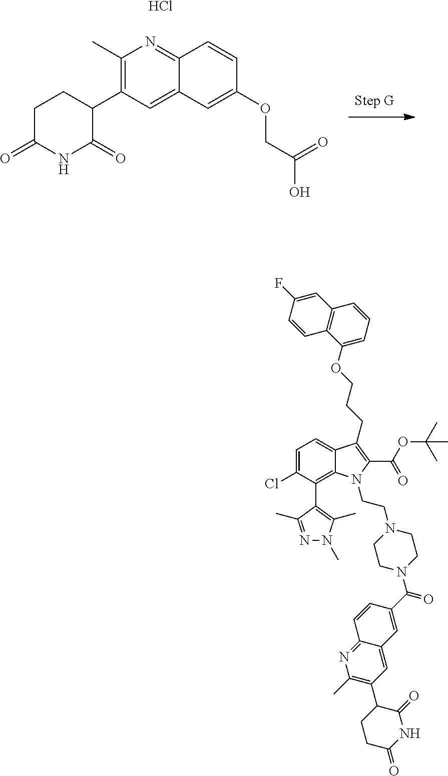 [SMILES: Cl ; Cc1nc2ccc(OCC(=O)O)cc2cc1C1CCC(=O)NC1=O ; Cc1nc2ccc(C(=O)N3CCN(CCn4c(C(=O)OC(C)(C)C)c(CCCOc5cccc6cc(F)ccc56)c5ccc(Cl)c(-c6c(C)nn(C)c6C)c54)CC3)cc2cc1C1CCC(=O)NC1=O]

To a solution of 2-{[3-(2,6-dioxopiperidin-3-yl)-2-methylquinolin-6-yl]oxy}acetic acid hydrochloride (5.8 mg, 0.016 mmol) and tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (10.0 mg, 0.015 mmol) in dry DMF (2.0 mL) were added HATU (11.3 mg, 0.030 mmol) and DIPEA (0.008 mL, 0.044 mmol). The reaction was stirred at room temperature for 15 min. The crude was diluted with DCM and washed with brine. The organic layer was dried over MgSO₄, filtered and concentrated in vacuo to obtain tert-butyl 6-chloro-1-{2-[4-(2-{[3-(2,6-dioxopiperidin-3-yl)-2-methylquinolin-6-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (45.9 mg, crude) as an yellow oil, which was used in the next step without further purification.

LCMS (ESI+): m/z 493.8 [M+2H]²⁺

### Step H

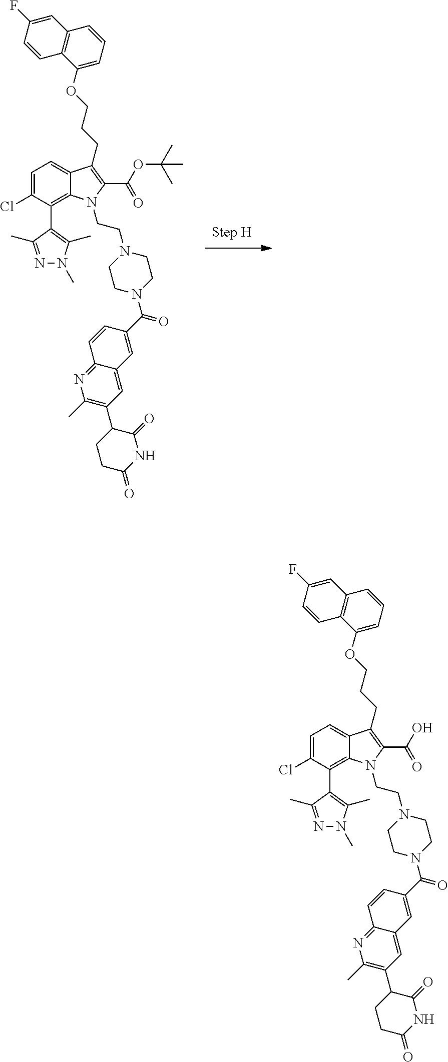 [SMILES: Cc1nc2ccc(C(=O)N3CCN(CCn4c(C(=O)OC(C)(C)C)c(CCCOc5cccc6cc(F)ccc56)c5ccc(Cl)c(-c6c(C)nn(C)c6C)c54)CC3)cc2cc1C1CCC(=O)NC1=O ; Cc1nc2ccc(C(=O)N3CCN(CCn4c(C(=O)O)c(CCCOc5cccc6cc(F)ccc56)c5ccc(Cl)c(-c6c(C)nn(C)c6C)c54)CC3)cc2cc1C1CCC(=O)NC1=O]

To a solution of tert-butyl 6-chloro-1-{2-[4-(2-{[3-(2,6-dioxopiperidin-3-yl)-2-methylquinolin-6-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (45.9 mg, crude) in DCM (1.0 mL) was added TFA (1.0 mL, 13.059 mmol). The reaction mixture was stirred for 18 h at RT. The crude was concentrated in vacuo and purified with preparative HPLC (C18, H₂O:MeCN+0.1% FA) to obtain 6-chloro-1-{2-[4-(2-{[3-(2,6-dioxopiperidin-3-yl)-2-methylquinolin-6-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (8.2 mg, 0.009 mmol, 56% over 2 steps) as a yellow solid.

LCMS (ESI+): m/z 928.15 [M+H]⁺

¹H NMR (500 MHz, DMSO, 353K) δ 10.58 (s, 1H), 8.24 (dd, J=9.2, 5.9 Hz, 1H), 7.92 (s, 1H), 7.82 (d, J=9.1 Hz, 1H), 7.67 (d, J=8.5 Hz, 1H), 7.58 (dd, J=10.4, 2.6 Hz, 1H), 7.43-7.39 (m, 2H), 7.36-7.29 (m, 2H), 7.22 (d, J=2.8 Hz, 1H), 7.19 (d, J=8.5 Hz, 1H), 6.87 (dd, J=5.6, 3.1 Hz, 1H), 4.84 (s, 2H), 4.32-4.16 (m, 5H), 3.74 (d, J=1.4 Hz, 3H), 3.41-3.36 (m, 4H), 3.28-3.24 (m, 2H), 2.80 (ddd, J=17.5, 12.3, 5.4 Hz, 1H), 2.64-2.59 (m, 4H), 2.39-2.30 (m, 1H), 2.26-2.21 (m, 2H), 2.18-2.04 (m, 7H), 2.00 (d, J=2.6 Hz, 3H), 1.88 (s, 3H).

### Example 136. 6-Chloro-1-(2-(4-(3-(2,6-dioxopiperidin-3-yl)-2-methylquinoline-6-carbonyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (290)

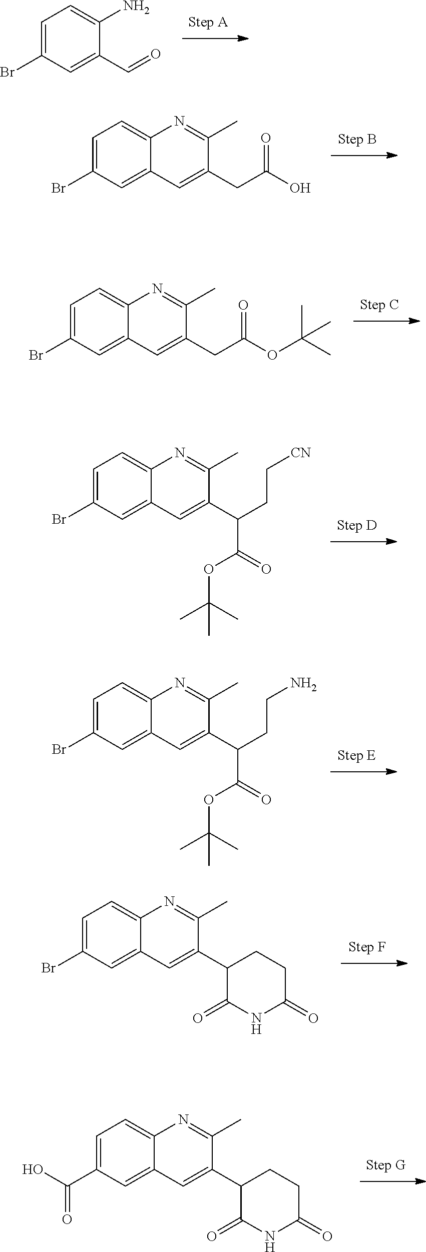

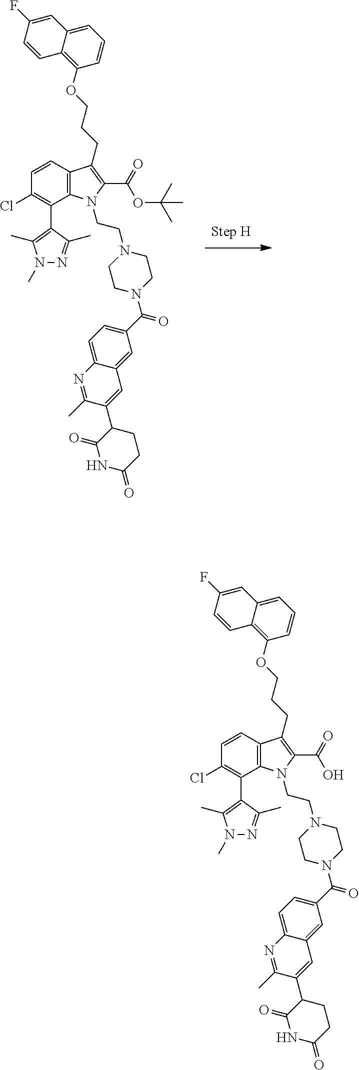 [SMILES: Cc1nc2ccc(C(=O)N3CCN(CCn4c(C(=O)OC(C)(C)C)c(CCCOc5cccc6cc(F)ccc56)c5ccc(Cl)c(-c6c(C)nn(C)c6C)c54)CC3)cc2cc1C1CCC(=O)NC1=O ; Cc1nc2ccc(C(=O)N3CCN(CCn4c(C(=O)O)c(CCCOc5cccc6cc(F)ccc56)c5ccc(Cl)c(-c6c(C)nn(C)c6C)c54)CC3)cc2cc1C1CCC(=O)NC1=O]

### Step A

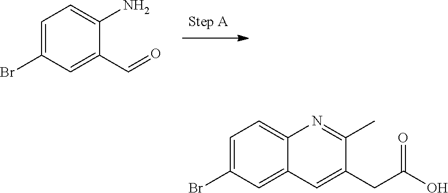 [SMILES: Nc1ccc(Br)cc1C=O ; Cc1nc2ccc(Br)cc2cc1CC(=O)O]

To a stirred solution of 2-amino-5-bromobenzaldehyde (5.8 g, 28.99 mmol) in MeOH (140 mL) was added 4-oxopentanoic acid (10.34 mL, 101.5 mmol) followed by 2M aqueous sodium hydroxide solution (20 mL) dropwise at 0° C. The resulting reaction mixture was allowed to reflux for 18 h. After complete consumption of the starting material, the reaction mixture was concentrated under reduced pressure. The residue was dissolved by minimum amount of water and it was washed with Et₂O (2-3 times). Then the aqueous layer was neutralized with acetic acid and the obtained precipitate was filtered, washed with ether and pentane to get 5 g of crude 2-(6-bromo-2-methylquinolin-3-yl)acetic acid as yellow solid which was directly used in the next step without further purification.

LCMS (ESI+): m/z 280.0 & 282.0 [M+H]⁺

### Step B

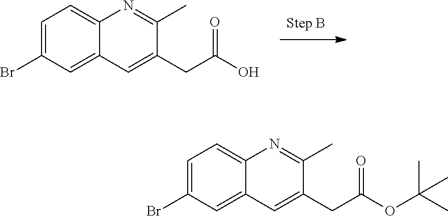 [SMILES: Cc1nc2ccc(Br)cc2cc1CC(=O)O ; Cc1nc2ccc(Br)cc2cc1CC(=O)OC(C)(C)C]

To a cooled solution of DCC (8.55 g, 41.43 mmol) in DCM (100 mL) was added DMAP (3.29 g, 26.93 mmol) at 0° C. followed by crude 2-(6-bromo-2-methylquinolin-3-yl)acetic acid (5 g) and the resulting mixture was stirred at 0° C. for 5 min. Then tert-butanol (3.071 mL, 103.57 mmol) was added to the reaction mixture and the resulting reaction mixture was allowed to stir at room temperature for 12 h under nitrogen. After complete consumption of the starting material, the solvent was evaporated under reduced pressure to get the crude compound. It was then diluted with EtOAc, washed successively with water and brine, organic layer was dried over Na₂SO₄ and evaporated under reduced pressure to get the crude compound which was purified with column chromatography (SiO₂, 30-40% EtOAc in hexane) to get tert-butyl 2-(6-bromo-2-methylquinolin-3-yl)acetate (4 g, 11.89 mmol, 41% over two steps) of as off white solid.

LCMS (ESI+): m/z 335.4 [M+H]⁺

### Step C

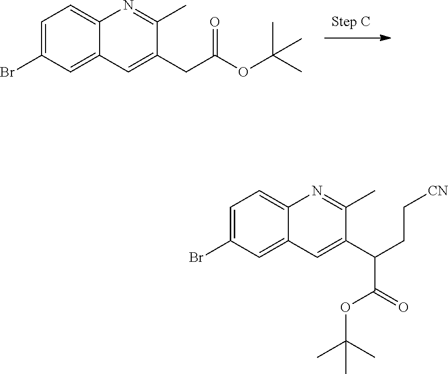 [SMILES: Cc1nc2ccc(Br)cc2cc1CC(=O)OC(C)(C)C ; Cc1nc2ccc(Br)cc2cc1C(CCC#N)C(=O)OC(C)(C)C]

To a stirred solution of tert-butyl 2-(6-bromo-2-methylquinolin-3-yl)acetate (4 g, 11.89 mmol) in DMF (50 mL) were added K₂CO₃ (1.81 g, 13.095 mmol) followed by TEBAC (2.712 g, 11.905 mmol) under nitrogen atmosphere at 0° C. Then acrylonitrile (0.858 mL, 13.095 mmol) was added into reaction mixture and the resultant reaction mixture was allowed to stir at room temperature for 3 h under nitrogen. After complete consumption of the starting material, the reaction mixture was diluted with EtOAc, washed successively with cold water and brine, the organic layer was dried over Na₂SO₄ and evaporated under reduced pressure to get the crude compound, which was then purified by column chromatography (SiO₂, 30-50% EtOAc in hexane) to get of tert-butyl 2-(6-bromo-2-methylquinolin-3-yl)-4-cyanobutanoate (1.4 g, 3.6 mmol, 30%) as light yellow sticky solid.

LCMS (ESI+): m/z 387.4 [M+H]⁺

### Step D

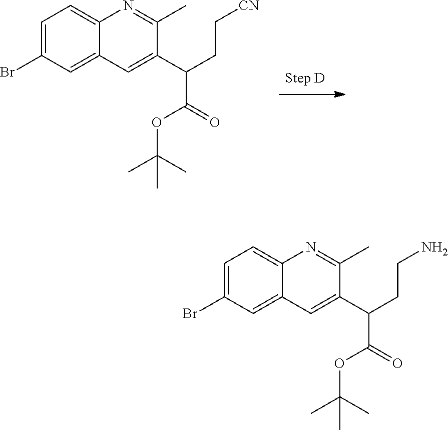 [SMILES: Cc1nc2ccc(Br)cc2cc1C(CCC#N)C(=O)OC(C)(C)C ; Cc1nc2ccc(Br)cc2cc1C(CCN)C(=O)OC(C)(C)C]

To a solution of tert-butyl 2-(6-bromo-2-methylquinolin-3-yl)-4-cyanobutanoate (1.4 g, 3.6 mmol) in MeOH (18 mL) were added H₂O₂ (1.5 mL, 3.6 mmol) followed by K₂CO₃ (995 mg, 7.209 mmol) under nitrogen atmosphere at 0° C. Then the resulting reaction mixture was allowed stir at room temperature for 16 h under nitrogen. After consumption of starting material, the reaction mixture was evaporated under vacuum to get the crude compound which was then purified by column chromatography (SiO₂, 2-3% MeOH in DCM) to get of tert-butyl 4-amino-2-(6-bromo-2-methylquinolin-3-yl)butanoate (700.0 mg, 1.85 mmol, 51%) as white solid.

### Step E

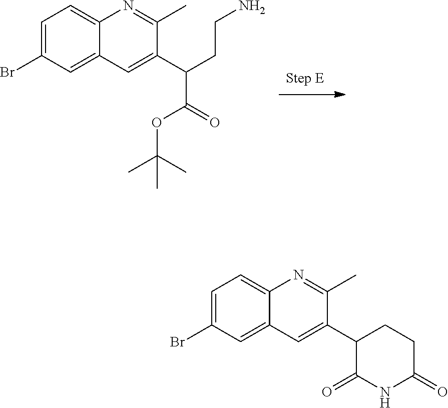 [SMILES: Cc1nc2ccc(Br)cc2cc1C(CCN)C(=O)OC(C)(C)C ; Cc1nc2ccc(Br)cc2cc1C1CCC(=O)NC1=O]

To stirred solution of tert-butyl 4-amino-2-(6-bromo-2-methylquinolin-3-yl)butanoate (700 mg, 1.85 mmol) in MeCN (10.0 mL) was added PTSA (980 g, 5.156 mmol) at room temperature and the reaction mixture was refluxed for 16 h. After complete consumption of the starting material, the reaction mixture was neutralized by adding Et₃N at 0° C. and then evaporated out to get crude compound which was then column chromatography (SiO₂, 5% MeOH in DCM) to get 3-(6-bromo-2-methylquinolin-3-yl)piperidine-2,6-dione (350 mg, 1.05 mmol, 57%) as a white solid.

LCMS (ESI+): m/z 333.1 & 335.0 [M+H]⁺

### Step F

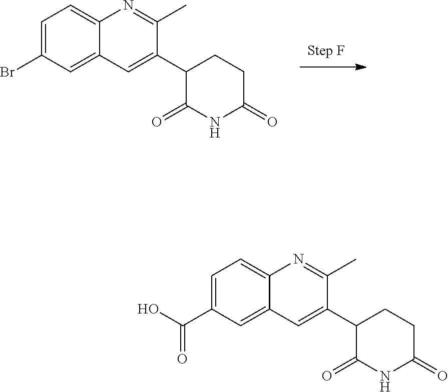 [SMILES: Cc1nc2ccc(Br)cc2cc1C1CCC(=O)NC1=O ; Cc1nc2ccc(C(=O)O)cc2cc1C1CCC(=O)NC1=O]

A solution of 3-(6-bromo-2-methylquinolin-3-yl)piperidine-2,6-dione (350.0 mg, 1.05 mmol) in DMF (5.0 mL), H₂O (3 mL), MeCN (5 mL), was added BINAP (65.34 mg, 0.105 mmol), Mo(CO)₆ (277.32 mg, 1.05 mmol) and CsF (160 mg, 1.05 mmol) successively at RT. The reaction mixture was de-oxygenated with argon for 10 minutes and then Pd(OAc)₂ (11.8 mg, 0.053 mmol) was added to it. The reaction mixture was allowed to stir at 90° C. for 16 hr. After complete consumption of the starting material, the reaction mixture was filtered through a Celite® pad and the filtrate was purified by reverse phase preparative HPLC (C18, H₂O:MeCN+0.1% FA) to get 3-(2,6-dioxopiperidin-3-yl)-2-methylquinoline-6-carboxylic acid (130 mg, 0.436 mmol, 41.5%) as white solid.

LCMS (ESI+): m/z 299.2 [M+H]⁺

### Step G

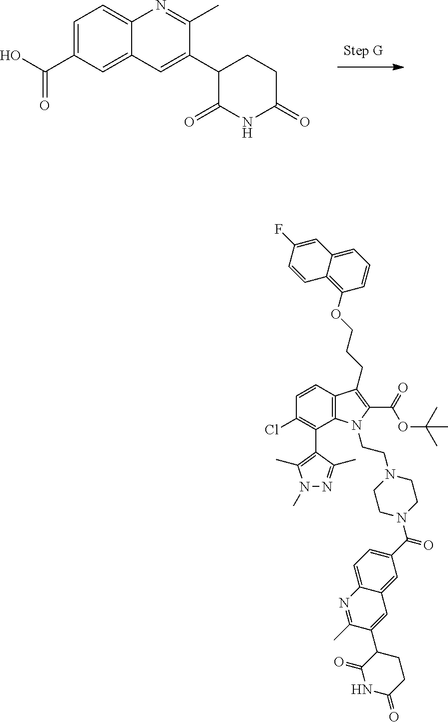 [SMILES: Cc1nc2ccc(C(=O)O)cc2cc1C1CCC(=O)NC1=O ; Cc1nc2ccc(C(=O)N3CCN(CCn4c(C(=O)OC(C)(C)C)c(CCCOc5cccc6cc(F)ccc56)c5ccc(Cl)c(-c6c(C)nn(C)c6C)c54)CC3)cc2cc1C1CCC(=O)NC1=O]

To a solution of 3-(2,6-dioxopiperidin-3-yl)-2-methylquinoline-6-carboxylic acid (25.0 mg, 0.037 mmol) in dry DMF (1.2 mL), was added DIPEA (0.019 mL, 0.111 mmol) and HATU (14.8 mg, 0.039 mmol). Reaction was stirred for 15 min under argon atmosphere and tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (25.0 mg, 0.037 mmol) was added. Reaction was stirred for 60 min. After complete consumption of the starting material, the solution was diluted with DCM and washed with brine and water. The organic layer was dried over anhydrous MgSO₄ and filtered. Resulting material was dried under reduced pressure to get crude tert-butyl 6-chloro-1-(2-{4-[3-(2,6-dioxopiperidin-3-yl)-2-methylquinoline-6-carbonyl]piperazin-1-yl}ethyl)-3-{3-[(6-fluoronaphthalen-1-yl)oxy]propyl}-7-(1,3,5-trimethyl-1H-pyrazol-4-yl)-1H-indene-2-carboxylate that was directly used in the next step.

LCMS (ESI+): m/z 954.4 [M+H]⁺

### Step H

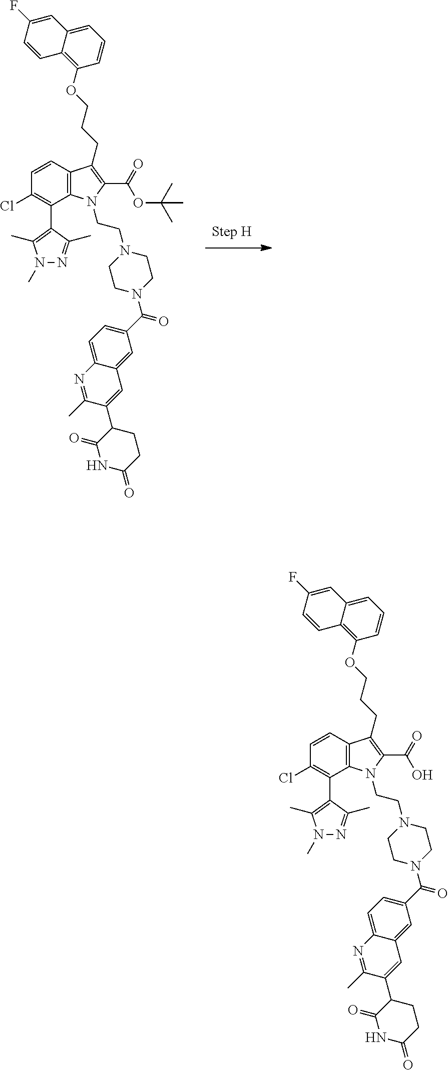 [SMILES: Cc1nc2ccc(C(=O)N3CCN(CCn4c(C(=O)OC(C)(C)C)c(CCCOc5cccc6cc(F)ccc56)c5ccc(Cl)c(-c6c(C)nn(C)c6C)c54)CC3)cc2cc1C1CCC(=O)NC1=O ; Cc1nc2ccc(C(=O)N3CCN(CCn4c(C(=O)O)c(CCCOc5cccc6cc(F)ccc56)c5ccc(Cl)c(-c6c(C)nn(C)c6C)c54)CC3)cc2cc1C1CCC(=O)NC1=O]

Crude tert-butyl 6-chloro-1-(2-{4-[3-(2,6-dioxopiperidin-3-yl)-2-methylquinoline-6-carbonyl]piperazin-1-yl}ethyl)-3-{3-[(6-fluoronaphthalen-1-yl)oxy]propyl}-7-(1,3,5-trimethyl-1H-pyrazol-4-yl)-1H-indene-2-carboxylate obtained in the previous step was dissolved in dry DCM (0.4 mL) under argon atmosphere and TFA (0.400 mL, 5.224 mmol) was added. Reaction was stirred at room temperature for 16 h. After complete consumption of the starting material, the solution was concentrated to dryness under reduced pressure, dissolved in DMSO and purified with preparative HPLC (C18, H₂O:MeCN+0.1% FA) to give 6-chloro-1-(2-{4-[3-(2,6-dioxopiperidin-3-yl)-2-methylquinoline-6-carbonyl]piperazin-1-yl}ethyl)-3-{3-[(6-fluoronaphthalen-1-yl)oxy]propyl}-7-(1,3,5-trimethyl-1H-pyrazol-4-yl)-1H-indene-2-carboxylic acid (18.2 mg, 0.020 mmol, 54% over 2 steps) as white solid.

LCMS (ESI+): m/z 898.15 [M+H]⁺

¹H NMR (500 MHz, DMSO, 353 K) δ 10.61 (s, 1H), 8.23 (dd, J=9.3, 5.9 Hz, 1H), 8.14-8.13 (m, 1H), 7.93 (d, J=8.6 Hz, 1H), 7.86 (d, J=1.9 Hz, 1H), 7.70 (d, J=8.5 Hz, 1H), 7.61 (dd, J=8.6, 1.9 Hz, 1H), 7.58 (dd, J=10.3, 2.6 Hz, 1H), 7.43-7.37 (m, 2H), 7.32 (td, J=8.9, 2.6 Hz, 1H), 7.21 (d, J=8.5 Hz, 1H), 6.86 (dd, J=6.4, 2.2 Hz, 1H), 4.34-4.14 (m, 5H), 3.73 (d, J=2.6 Hz, 3H), 3.49-3.34 (m, 4H), 3.32-3.25 (m, 2H), 2.83 (ddd, J=17.6, 12.5, 5.4 Hz, 1H), 2.69 (s, 3H), 2.68-2.61 (m, 1H), 2.44-2.32 (m, 1H), 2.27-2.20 (m, 2H), 2.20-2.07 (m, 7H), 2.01 (s, 3H), 1.88 (s, 3H).

### Example 137. 6-Chloro-1-(2-{4-[3-(2,6-dioxopiperidin-3-yl)-2-methylquinoline-7-carbonyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylic acid (291)

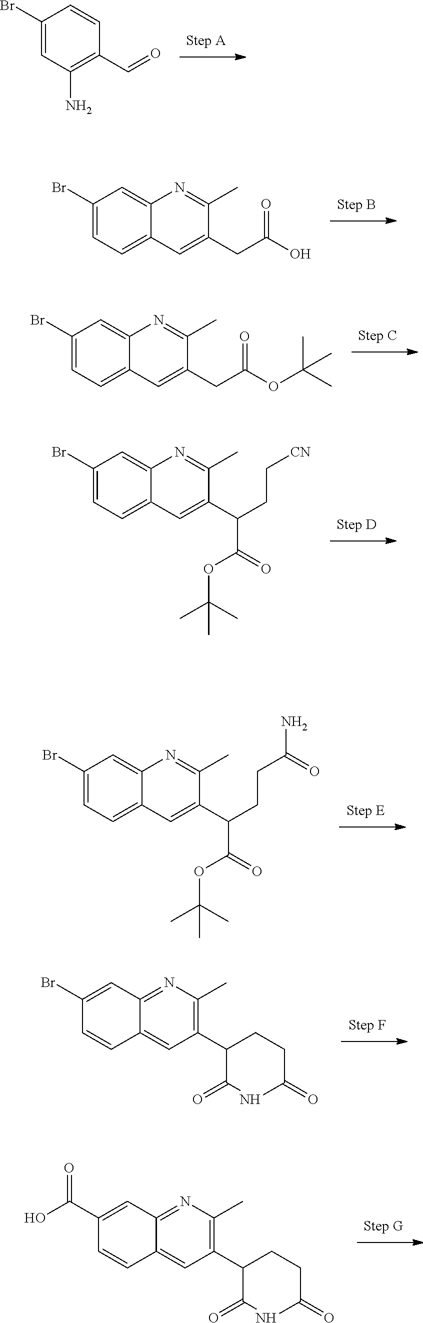

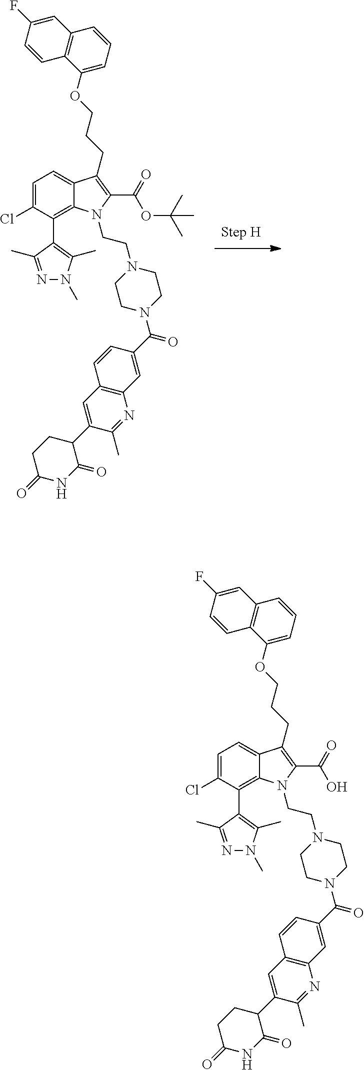 [SMILES: Cc1nc2cc(C(=O)N3CCN(CCn4c(C(=O)OC(C)(C)C)c(CCCOc5cccc6cc(F)ccc56)c5ccc(Cl)c(-c6c(C)nn(C)c6C)c54)CC3)ccc2cc1C1CCC(=O)NC1=O ; Cc1nc2cc(C(=O)N3CCN(CCn4c(C(=O)O)c(CCCOc5cccc6cc(F)ccc56)c5ccc(Cl)c(-c6c(C)nn(C)c6C)c54)CC3)ccc2cc1C1CCC(=O)NC1=O]

### Step A

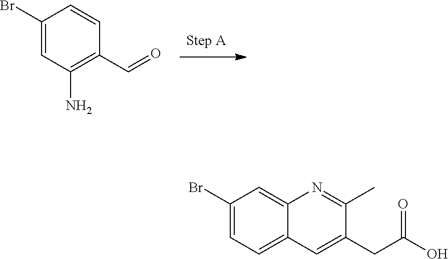 [SMILES: Nc1cc(Br)ccc1C=O ; Cc1nc2cc(Br)ccc2cc1CC(=O)O]

To a solution of 2-amino-4-bromobenzaldehyde (6.0 g, 29.99 mmol) in MeOH (100 mL) was added 4-oxopentanoic acid (5.24 g, 44.99 mmol) followed by 2 m aqueous sodium hydroxide solution (24 mL) dropwise at room temperature. Then, the resultant reaction mixture was allowed to reflux for 16 h. After complete consumption of the starting material (monitored by LCMS) the reaction mixture was concentrated under reduced pressure. Then it was neutralized with acetic acid and the obtained precipitate was filtered, washed with ether and pentane to get 2-(7-bromo-2-methylquinolin-3-yl)acetic acid (8.0 g, 28.57 mmol 95%) as pale yellow sticky solid.

LCMS (ESI+): m/z 280.0 [M+H]⁺

### Step B

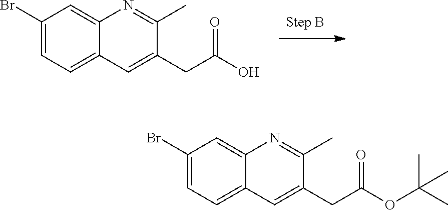 [SMILES: Cc1nc2cc(Br)ccc2cc1CC(=O)O ; Cc1nc2cc(Br)ccc2cc1CC(=O)OC(C)(C)C]

To a cooled solution of DCC (11.83 g, 57.35 mmol) in DCM (150 mL) was added DMAP (4.55 g, 37.27 mmol) at 0° C. followed by 2-(7-bromo-2-methylquinolin-3-yl)acetic acid (8.0 g, 28.67 mmol) and the resulting mixture was stirred at 0° C. for 5 min. Then tert-butanol (4.25 mL, 143.37 mmol) was added and the resulting reaction mixture was allowed to stir at room temperature for 16 h under nitrogen. After complete consumption of the starting material (monitored by LCMS), the solvent of was evaporated under reduced pressure to get the crude compound. It was then diluted with EtOAc and washed successively with water and brine. Organic layer was dried over anhydrous Na₂SO₄ and evaporated under reduced pressure to get the crude compound, which was then purified by column chromatography (SiO₂, 25% EtOAc in hexane) to obtain tert-butyl 2-(7-bromo-2-methylquinolin-3-yl)acetate (5.0 g, 14.88 mmol, 52%) as white sticky solid.

LCMS (ESI+): m/z 335.3 [M+H]⁺

### Step C

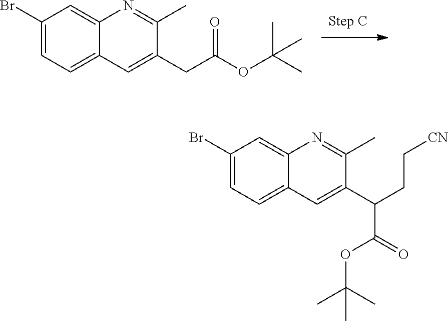 [SMILES: Cc1nc2cc(Br)ccc2cc1CC(=O)OC(C)(C)C ; Cc1nc2cc(Br)ccc2cc1C(CCC#N)C(=O)OC(C)(C)C]

To a solution of tert-butyl 2-(7-bromo-2-methylquinolin-3-yl)acetate (5.0 g, 14.88 mmol) in DMF (250 mL) was added K₂CO₃ (2 g, 14.88 mmol) followed by TEBAC (3.4 g, 14.88 mmol) under nitrogen atmosphere at 0° C. Then MeCN (0.8 mL, 14.88 mmol) added into reaction mixture at RT. Then the resulting reaction mixture was allowed to stir at room temperature for 4 h under nitrogen. After complete consumption of the starting material (monitored by LCMS) the reaction mixture was diluted with EtOAc and washed successively with cold water and brine. The organic layer was dried over anhydrous Na₂SO₄ and evaporated under reduced pressure to get the crude compound, which was then purified with column chromatography (SiO₂, 20-25% EtOAc in hexane) to get tert-butyl 2-(7-bromo-2-methylquinolin-3-yl)-4-cyanobutanoate (2.7 g, 6.94 mmol, 47%) as white sticky liquid.

LCMS (ESI+): m/z 390.8 [M+H]⁺

### Step D

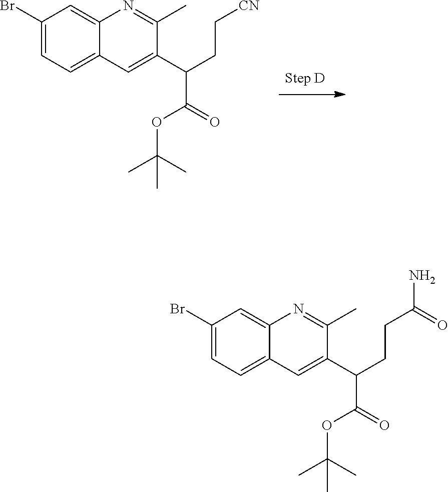 [SMILES: Cc1nc2cc(Br)ccc2cc1C(CCC#N)C(=O)OC(C)(C)C ; Cc1nc2cc(Br)ccc2cc1C(CCC(N)=O)C(=O)OC(C)(C)C]

To a stirred solution of tert-butyl 2-(7-bromo-2-methylquinolin-3-yl)-4-cyanobutanoate (2.7 g, 6.94 mmol) in MeOH (100 mL) was added H₂O₂ (1.061 mL, 31.21 mmol) followed by K₂CO₃ (0.191 g, 1.39 mmol) under nitrogen atmosphere at 0° C. Then the resulting reaction mixture was allowed stir at room temperature for 16 h under nitrogen. After complete consumption of the starting material, the solvent was removed under reduced pressure to obtain tert-butyl 5-amino-2-(7-bromo-2-methylquinolin-3-yl)-5-oxopentanoate (2.7 g, crude) which was directly used in the next step without further purification.

LCMS (ESI+): m/z 407.1 [M+H]⁺

### Step E

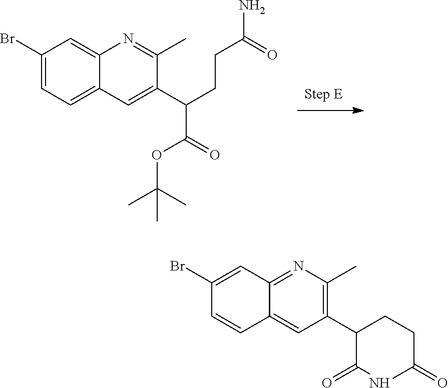 [SMILES: Cc1nc2cc(Br)ccc2cc1C(CCC(N)=O)C(=O)OC(C)(C)C ; Cc1nc2cc(Br)ccc2cc1C1CCC(=O)NC1=O]

Crude tert-butyl 5-amino-2-(7-bromo-2-methylquinolin-3-yl)-5-oxopentanoate (2.7 g, crude) was dissolved in 150.0 mL MeCN and p-toluene sulfonic acid monohydrate (4.56 mg, 26.515 mmol) was added and then the resulting reaction mixture was allowed to heat at 80° C. for 16 h. After reaction completion (monitored LCMS), reaction mixture was concentrated under reduced pressure and diluted with EtOAc and filtered. The obtained crude product was purified with column chromatography (SiO₂, 40-45% EtOAc in DCM) to afford 3-(7-bromo-2-methylquinolin-3-yl)piperidine-2,6-dione (1.5 g, 4.50 mmol, 64.8% over 2 steps) as white solid.

LCMS (ESI+): m/z 334.9 [M+H]⁺

### Step F

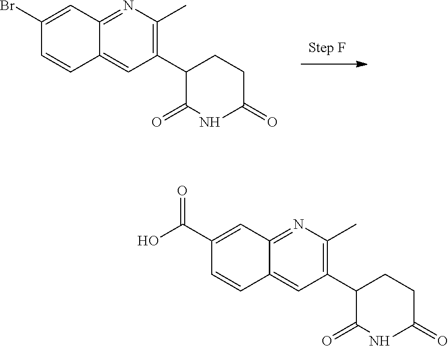 [SMILES: Cc1nc2cc(Br)ccc2cc1C1CCC(=O)NC1=O ; Cc1nc2cc(C(=O)O)ccc2cc1C1CCC(=O)NC1=O]

A solution of 3-(7-bromo-2-methylquinolin-3-yl)piperidine-2,6-dione (1.0 g, 3.01 mmol), Mo(CO)₆ (0.795 g, 3.01 mmol) in MeCN (10 mL), DMF (10 mL) and water (8 mL) was added CsF (0.46 g, 3.01 mmol). The reaction mixture was deoxygenated with argon stream for 10 min. BINAP (0.19 g, 0.30 mmol) followed by Pd(OAc)₂ (0.034 g, 0.15 mmol) were added to the reaction mixture and it was allowed to stir at 90° C. for 16 h. After complete consumption of the starting material, the reaction mixture was filtered through Celite® and washed with MeCN then concentrated under reduced pressure to get crude material which was purified with preparative HPLC (C18, 10 mM ammonium acetate buffer:MeCN) to obtain 3-(2,6-dioxopiperidin-3-yl)-2-methylquinoline-7-carboxylic acid (325 mg, 1.1 mmol, 36.5%) as off white solid.

LCMS (ESI+): m/z 299.0 [M+H]⁺

### Step G

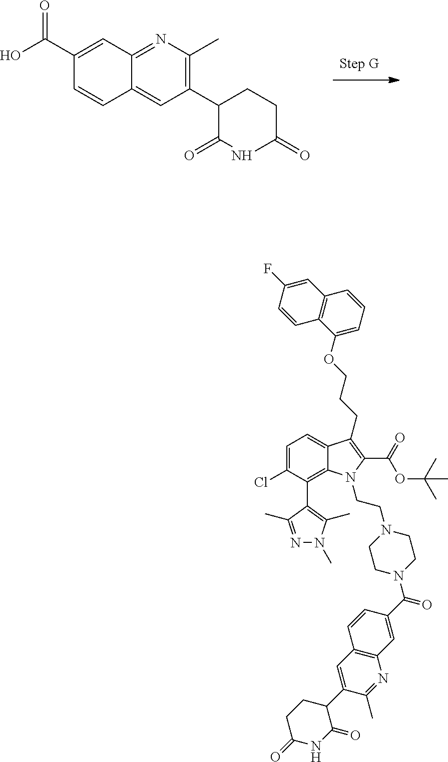 [SMILES: Cc1nc2cc(C(=O)O)ccc2cc1C1CCC(=O)NC1=O ; Cc1nc2cc(C(=O)N3CCN(CCn4c(C(=O)OC(C)(C)C)c(CCCOc5cccc6cc(F)ccc56)c5ccc(Cl)c(-c6c(C)nn(C)c6C)c54)CC3)ccc2cc1C1CCC(=O)NC1=O]

To a solution of tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (20.0 mg, 0.030 mmol) and 3-(2,6-dioxopiperidin-3-yl)-2-methylquinoline-7-carboxylic acid (10.6 mg, 0.036 mmol) in dry DMF (2.0 mL) were added HATU (22.6 mg, 0.059 mmol) and DIPEA (0.026 mL, 0.148 mmol). The reaction was stirred at room temperature for 30 min. The crude was diluted in DCM and washed with brine. The organic layer was dried over MgSO₄, filtered and concentrated in vacuo to obtain tert-butyl 6-chloro-1-(2-{4-[3-(2,6-dioxopiperidin-3-yl)-2-methylquinoline-7-carbonyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate (50 mg, crude) as yellow oil, which was used in the next step without further purification.

LCMS (ESI+): m/z 954.4 [M+H]⁺

### Step H

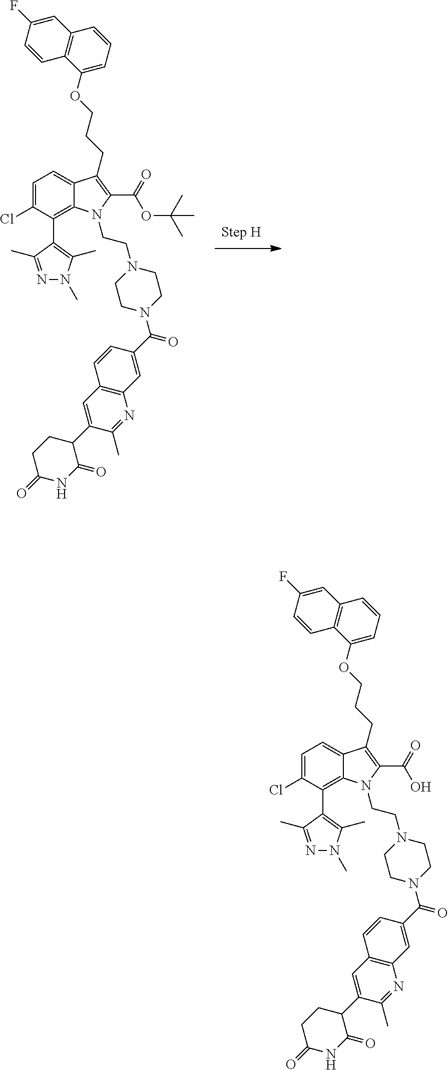 [SMILES: Cc1nc2cc(C(=O)N3CCN(CCn4c(C(=O)OC(C)(C)C)c(CCCOc5cccc6cc(F)ccc56)c5ccc(Cl)c(-c6c(C)nn(C)c6C)c54)CC3)ccc2cc1C1CCC(=O)NC1=O ; Cc1nc2cc(C(=O)N3CCN(CCn4c(C(=O)O)c(CCCOc5cccc6cc(F)ccc56)c5ccc(Cl)c(-c6c(C)nn(C)c6C)c54)CC3)ccc2cc1C1CCC(=O)NC1=O]

To a solution of tert-butyl 6-chloro-1-(2-{4-[3-(2,6-dioxopiperidin-3-yl)-2-methylquinoline-7-carbonyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate (50 mg, crude) in DCM (1.0 mL) was added TFA (1.0 mL) and the mixture was stirred for next 18 h at RT. The crude was concentrated in vacuo and purified with preparative HPLC (C18, H₂O:MeCN+0.1% FA) to obtain 6-chloro-1-(2-{4-[3-(2,6-dioxopiperidin-3-yl)-2-methylquinoline-7-carbonyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylic acid (12.75 mg, 0.014 mmol, 46.7% over 2 steps) as white solid.

LCMS (ESI+): m/z 898.25 [M+H]⁺

¹H NMR (500 MHz, DMSO, 353 K) δ 12.73 (s, 1H), 10.61 (s, 1H), 8.23 (dd, J=9.3, 5.9 Hz, 1H), 8.12 (s, 1H), 7.92 (d, J=8.3 Hz, 1H), 7.82 (s, 1H), 7.69 (d, J=8.5 Hz, 1H), 7.58 (dd, J=10.4, 2.7 Hz, 1H), 7.44 (dd, J=8.4, 1.6 Hz, 1H), 7.42-7.38 (m, 2H), 7.32 (td, J=8.9, 2.6 Hz, 1H), 7.20 (d, J=8.5 Hz, 1H), 6.86 (dd, J=6.4, 2.3 Hz, 1H), 4.32-4.26 (m, 2H), 4.24 (t, J=6.3 Hz, 2H), 4.22-4.16 (m, 1H), 3.75 (s, 3H), 3.41 (bs, 4H), 3.29-3.26 (m, 2H), 2.83 (ddd, J=17.6, 12.6, 5.4 Hz, 1H), 2.69 (s, 3H), 2.68-2.62 (m, 1H), 2.45-2.36 (m, 1H), 2.27-2.21 (m, 2H), 2.20-2.10 (m, 7H), 2.02 (s, 3H), 1.88 (s, 3H).

### Example 138. 6-Chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(2-methyl-6,7-dihydro-4H-pyrazolo[5,1-c][1,4]oxazin-3-yl)-1H-indole-2-carboxylic acid (346)

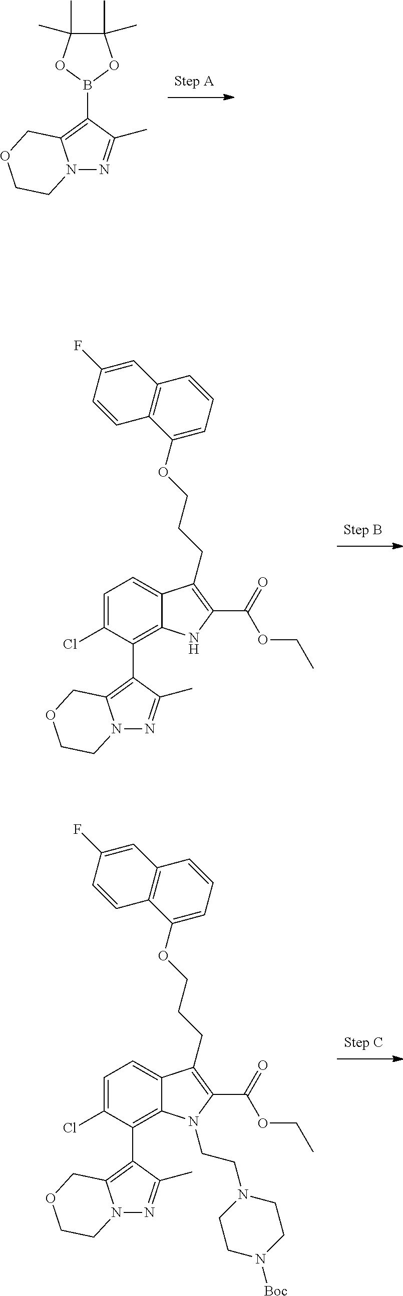 [SMILES: Cc1nn2c(c1B1OC(C)(C)C(C)(C)O1)COCC2 ; CCOC(=O)c1[nH]c2c(-c3c(C)nn4c3COCC4)c(Cl)ccc2c1CCCOc1cccc2cc(F)ccc12 ; CCOC(=O)c1c(CCCOc2cccc3cc(F)ccc23)c2ccc(Cl)c(-c3c(C)nn4c3COCC4)c2n1CCN1CCN(C(=O)OC(C)(C)C)CC1]

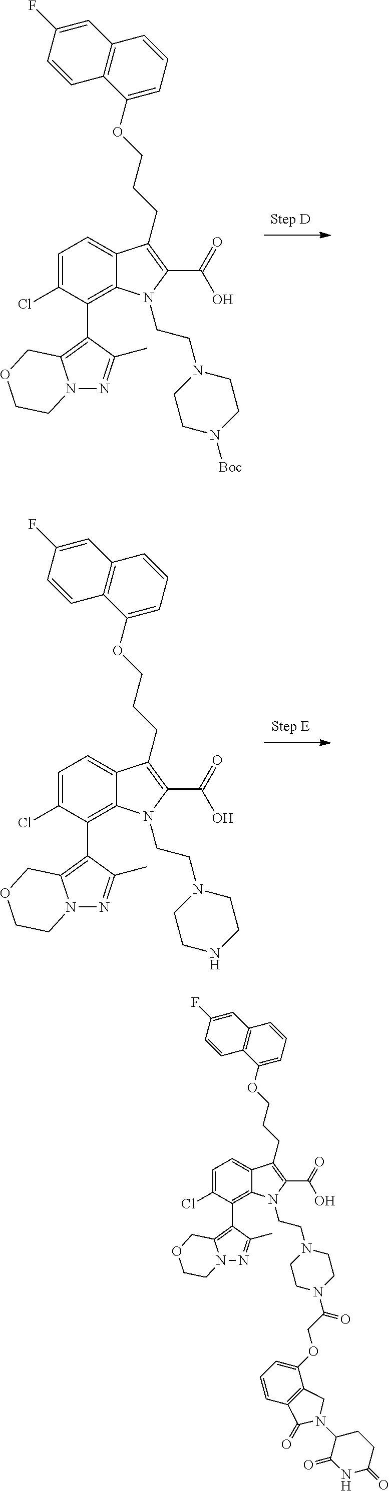 [SMILES: Cc1nn2c(c1-c1c(Cl)ccc3c(CCCOc4cccc5cc(F)ccc45)c(C(=O)O)n(CCN4CCN(C(=O)OC(C)(C)C)CC4)c13)COCC2 ; Cc1nn2c(c1-c1c(Cl)ccc3c(CCCOc4cccc5cc(F)ccc45)c(C(=O)O)n(CCN4CCNCC4)c13)COCC2 ; Cc1nn2c(c1-c1c(Cl)ccc3c(CCCOc4cccc5cc(F)ccc45)c(C(=O)O)n(CCN4CCN(C(=O)COc5cccc6c5CN(C5CCC(=O)NC5=O)C6=O)CC4)c13)COCC2]

### Step A

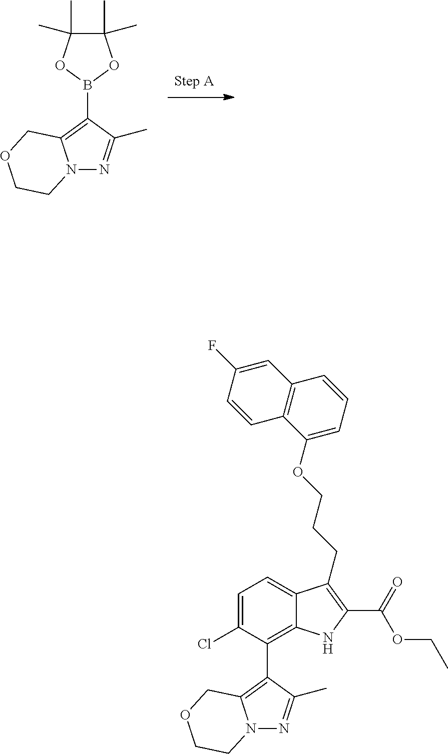 [SMILES: Cc1nn2c(c1B1OC(C)(C)C(C)(C)O1)COCC2 ; CCOC(=O)c1[nH]c2c(-c3c(C)nn4c3COCC4)c(Cl)ccc2c1CCCOc1cccc2cc(F)ccc12]

To a solution of ethyl 7-bromo-6-chloro-3-(3-((6-fluoronaphthalen-1-yl)oxy)propyl)-1H-indole-2-carboxylate (150 mg, 0.30 mmol) in dioxane (3.6 mL) and water (0.9 mL), were added 2-methyl-3-(4,4,5,5-tetramethyl-1,3,2-dioxaborolan-2-yl)-6,7-dihydro-4H-pyrazolo[5,1-c][1,4]oxazine (154 mg, 0.58 mmol) and K₃PO₄ (184 mg, 0.872 mmol) at room temperature under nitrogen. The mixture was deoxygenated with argon and to it was added PdCl₂(dtbpf) (38 mg, 0.058 mmol) under argon atmosphere. Then, the reaction mixture was heated under reflux for 16 h. After complete consumption of the starting material (monitored by TLC and LCMS), the reaction mixture was filtered through Celite® pad and the filtrate was evaporated under reduced pressure to get the residue. It was then diluted with EtOAc and washed successively with water and brine. The organic layer was dried over Na₂SO₄ and evaporated under reduced pressure to get the crude compound, which was then purified by column chromatography (SiO₂, 20-30% EtOAc in hexane) to get ethyl 6-chloro-3-(3-((6-fluoronaphthalen-1-yl)oxy)propyl)-7-(2-methyl-6,7-dihydro-4H-pyrazolo[5,1-c][1,4]oxazin-3-yl)-1H-indole-2-carboxylate (120 mg, 0.213 mmol, 71%) as brown solid.

LCMS (ESI+): m/z 562.3 [M+H]⁺

### Step B

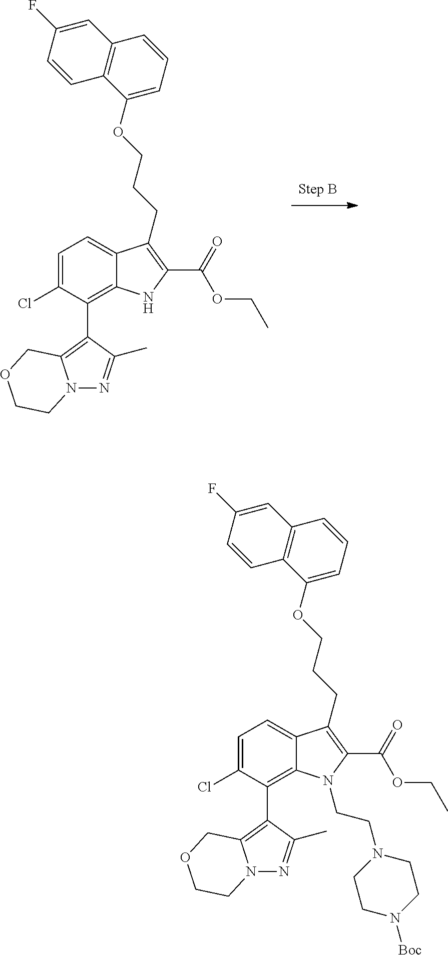 [SMILES: CCOC(=O)c1[nH]c2c(-c3c(C)nn4c3COCC4)c(Cl)ccc2c1CCCOc1cccc2cc(F)ccc12 ; CCOC(=O)c1c(CCCOc2cccc3cc(F)ccc23)c2ccc(Cl)c(-c3c(C)nn4c3COCC4)c2n1CCN1CCN(C(=O)OC(C)(C)C)CC1]

To a solution of ethyl 6-chloro-3-(3-((6-fluoronaphthalen-1-yl)oxy)propyl)-7-(2-methyl-6,7-dihydro-4H-pyrazolo[5,1-c][1,4]oxazin-3-yl)-1H-indole-2-carboxylate (120 mg, 0.213 mmol) in DMF (2 mL) was added tert-butyl 4-(2-chloroethyl)piperazine-1-carboxylate (70 mg, 0.281 mmol) followed by Cs₂CO₃ (104 mg, 0.32 mmol) at room temperature under nitrogen. The resulting mixture was allowed to stir at 90° C. for 16 h under nitrogen. After complete consumption of the starting material (monitored by TLC and LCMS), the reaction mixture was diluted with EtOAc and washed successively with water and brine. The organic layer was dried over Na₂SO₄ and evaporated under reduced pressure to get 150 mg of crude ethyl 1-(2-(4-(tert-butoxycarbonyl)piperazin-1-yl)ethyl)-6-chloro-3-(3-((6-fluoronaphthalen-1-yl)oxy)propyl)-7-(2-methyl-6,7-dihydro-4H-pyrazolo[5,1-c][1,4]oxazin-3-yl)-1H-indole-2-carboxylate as brown solid, which was then directly used in the next step without further purification.

LCMS (ESI+): m/z 774.0 [M+H]⁺

### Step C

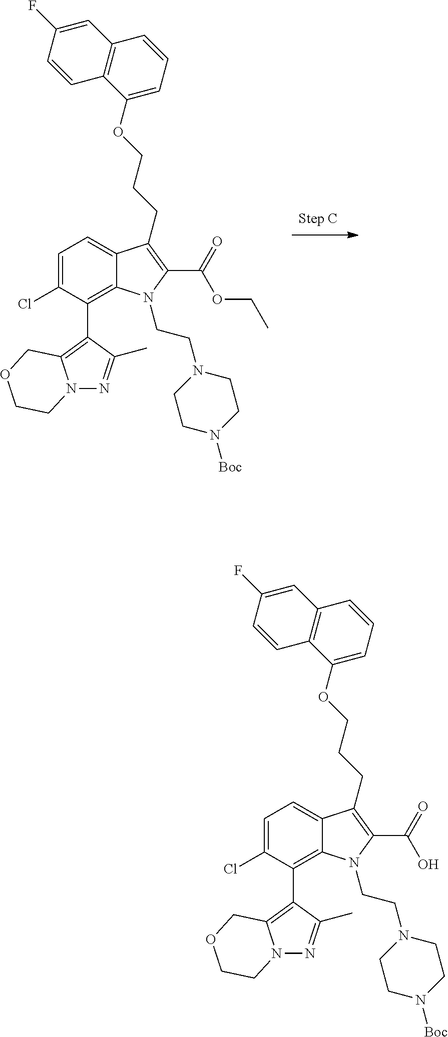 [SMILES: CCOC(=O)c1c(CCCOc2cccc3cc(F)ccc23)c2ccc(Cl)c(-c3c(C)nn4c3COCC4)c2n1CCN1CCN(C(=O)OC(C)(C)C)CC1 ; Cc1nn2c(c1-c1c(Cl)ccc3c(CCCOc4cccc5cc(F)ccc45)c(C(=O)O)n(CCN4CCN(C(=O)OC(C)(C)C)CC4)c13)COCC2]

Crude ethyl 1-(2-(4-(tert-butoxycarbonyl)piperazin-1-yl)ethyl)-6-chloro-3-(3-((6-fluoronaphthalen-1-yl)oxy)propyl)-7-(2-methyl-6,7-dihydro-4H-pyrazolo[5,1-c][1,4]oxazin-3-yl)-1H-indole-2-carboxylate (150 mg) was dissolved in EtOH (4 mL) and a solution of NaOH (30 mg, 0.78 mmol) in water (1 mL) was added to it. The mixture was heated under reflux for 16 h. After complete consumption of the starting material (monitored by TLC and LCMS), the reaction mixture was cooled down to room temperature and solvents were evaporated under reduced pressure. It was then diluted with water and washed with EtOAc. Aqueous layer was carefully acidified using aqueous 1M HCl to pH=3, extracted with EtOAc, dried over Na₂SO₄, filtered, and concentrated in vacuo. Crude 1-(2-(4-(tert-butoxycarbonyl)piperazin-1-yl)ethyl)-6-chloro-3-(3-((6-fluoronaphthalen-1-yl)oxy)propyl)-7-(2-methyl-6,7-dihydro-4H-pyrazolo[5,1-c][1,4]oxazin-3-yl)-1H-indole-2-carboxylic acid (70 mg) was obtained as dark brown gummy solid which was directly used in the next step without further purification

LCMS (ESI+): m/z 746.1 [M+H]⁺

### Step D

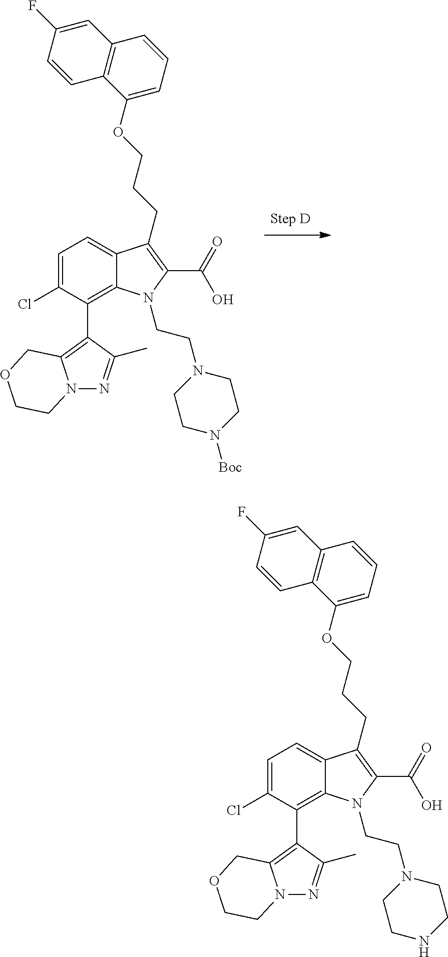 [SMILES: Cc1nn2c(c1-c1c(Cl)ccc3c(CCCOc4cccc5cc(F)ccc45)c(C(=O)O)n(CCN4CCN(C(=O)OC(C)(C)C)CC4)c13)COCC2 ; Cc1nn2c(c1-c1c(Cl)ccc3c(CCCOc4cccc5cc(F)ccc45)c(C(=O)O)n(CCN4CCNCC4)c13)COCC2]

Crude 1-(2-(4-(tert-butoxycarbonyl)piperazin-1-yl)ethyl)-6-chloro-3-(3-((6-fluoronaphthalen-1-yl)oxy)propyl)-7-(2-methyl-6,7-dihydro-4H-pyrazolo[5,1-c][1,4]oxazin-3-yl)-1H-indole-2-carboxylic acid (70 mg) was dissolved in 4M HCl in dioxane (2 mL) at 0° C. and the mixture stirred for 2 h under nitrogen at RT. Where upon LCMS indicated the reaction was complete, the volatiles were concentrated in vacuum to get the crude compound which was then purified by reverse phase preparative HPLC (H₂O (20 mM ammonium bicarbonate solution):MeCN) to get 6-chloro-3-(3-((6-fluoronaphthalen-1-yl)oxy)propyl)-7-(2-methyl-6,7-dihydro-4H-pyrazolo[5,1-c][1,4]oxazin-3-yl)-1-(2-(piperazin-1-yl)ethyl)-1H-indole-2-carboxylic acid (25 mg, 0.039 mmol, 18% over 3 steps) as white solid.

LCMS (ESI+): m/z 646.4 [M+H]⁺

### Step E

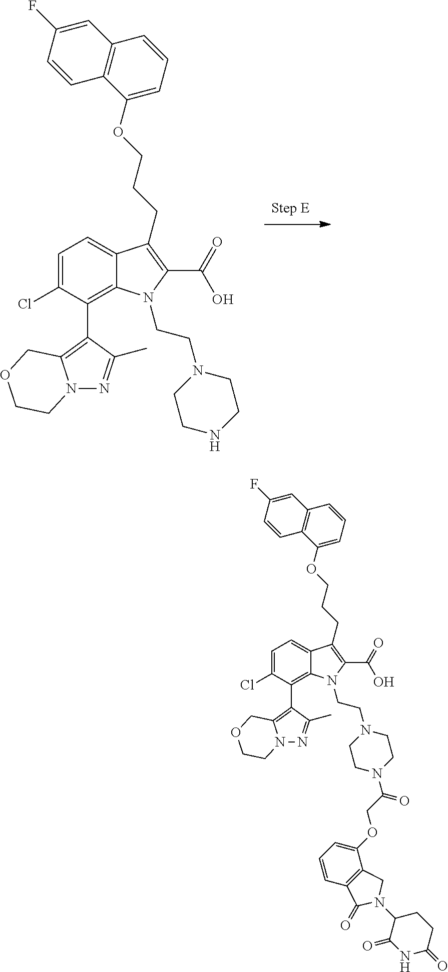 [SMILES: Cc1nn2c(c1-c1c(Cl)ccc3c(CCCOc4cccc5cc(F)ccc45)c(C(=O)O)n(CCN4CCNCC4)c13)COCC2 ; Cc1nn2c(c1-c1c(Cl)ccc3c(CCCOc4cccc5cc(F)ccc45)c(C(=O)O)n(CCN4CCN(C(=O)COc5cccc6c5CN(C5CCC(=O)NC5=O)C6=O)CC4)c13)COCC2]

2-{[2-(2,6-Dioxopiperidin-3-yl)-1-oxo-2,3-dihydro-1H-isoindol-4-yl]oxy}acetic acid (30.6 mg, 0.096 mmol) was dissolved in dry DMF (6.2 mL) under argon atmosphere and DIPEA (0.025 mL, 0.144 mmol), followed by HATU (32.9 mg, 0.087 mmol) were added. The solution was stirred at room temperature for 1 h under argon. 6-chloro-3-(3-((6-fluoronaphthalen-1-yl)oxy)propyl)-7-(2-methyl-6,7-dihydro-4H-pyrazolo[5,1-c][1,4]oxazin-3-yl)-1-(2-(piperazin-1-yl)ethyl)-1H-indole-2-carboxylic acid (31.1 mg, 0.048 mmol) and DIPEA (0.025 mL, 0.144 mmol) were dissolved in dry DMSO (1.6 mL) and added dropwise to the reaction, which was then continued for the next 20 min at room temperature under argon atmosphere and monitored with LCMS. After complete consumption of the starting material, the solution was concentrated under reduced pressure, diluted with DMSO and the crude product was purified with preparative HPLC (C18, H₂O:MeCN+0.1% FA) to get 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(2-methyl-6,7-dihydro-4H-pyrazolo[5,1-c][1,4]oxazin-3-yl)-1H-indole-2-carboxylic acid (33.7 mg, 0.036 mmol, 74.0%) as white powder.

LCMS (ESI+): m/z 946.15 [M+H]⁺

¹H NMR (500 MHz, DMSO, 353 K) δ 10.64 (s, 1H), 8.23 (dd, J=9.2, 5.9 Hz, 1H), 7.73 (d, J=8.5 Hz, 1H), 7.58 (dd, J=10.4, 2.6 Hz, 1H), 7.48-7.37 (m, 3H), 7.36-7.28 (m, 2H), 7.22 (d, J=8.6 Hz, 1H), 7.14 (dd, J=8.2, 0.8 Hz, 1H), 6.87 (dd, J=5.7, 3.0 Hz, 1H), 5.04 (dd, J=13.1, 5.2 Hz, 1H), 4.89 (s, 2H), 4.54 (d, J=15.0 Hz, 1H), 4.50 (d, J=15.2 Hz, 1H), 4.40 (d, J=17.1 Hz, 1H), 4.37-4.28 (m, 2H), 4.24 (t, J=6.2 Hz, 2H), 4.21-4.13 (m, 2H), 4.13-4.04 (m, 3H), 3.40-3.32 (m, 4H), 3.32-3.25 (m, 2H), 2.88 (ddd, J=17.4, 13.4, 5.5 Hz, 1H), 2.66-2.59 (m, 1H), 2.43 (td, J=13.1, 4.6 Hz, 1H), 2.28-2.20 (m, 2H), 2.20-2.09 (m, 6H), 2.05 (dtd, J=12.8, 5.4, 2.6 Hz, 1H), 1.93 (s, 3H).

### Example 139. 6-Chloro-7-(3,5-dimethylisoxazol-4-yl)-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-1H-indole-2-carboxylic acid (347)

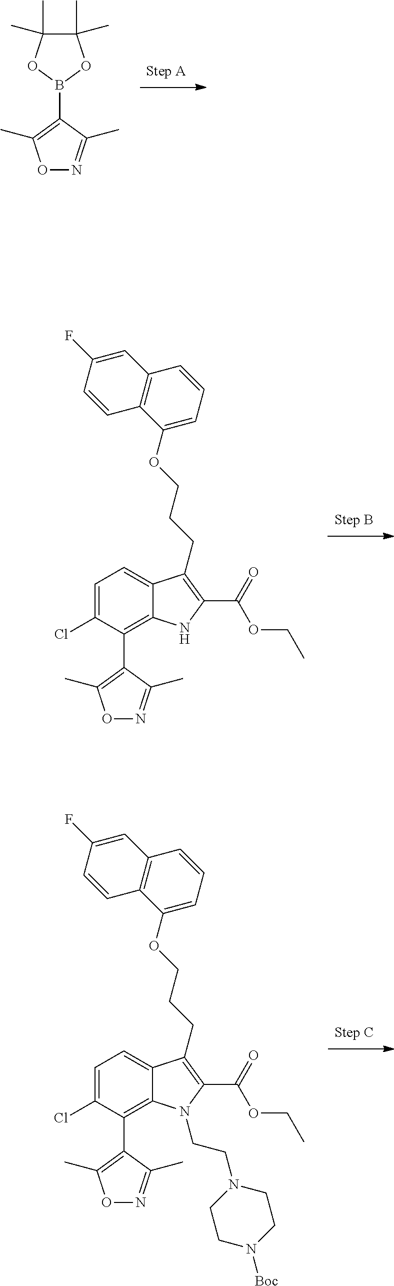

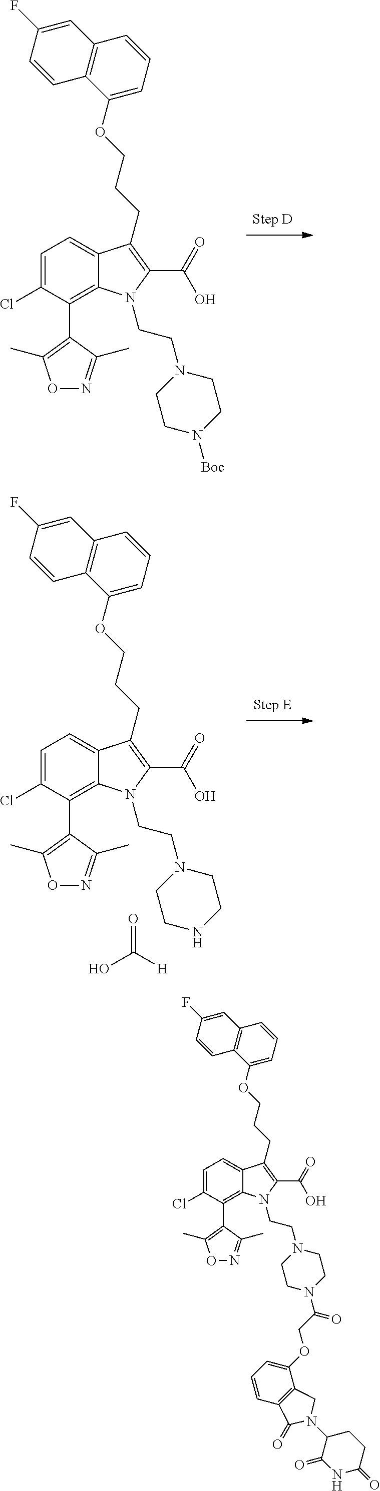

### Step A

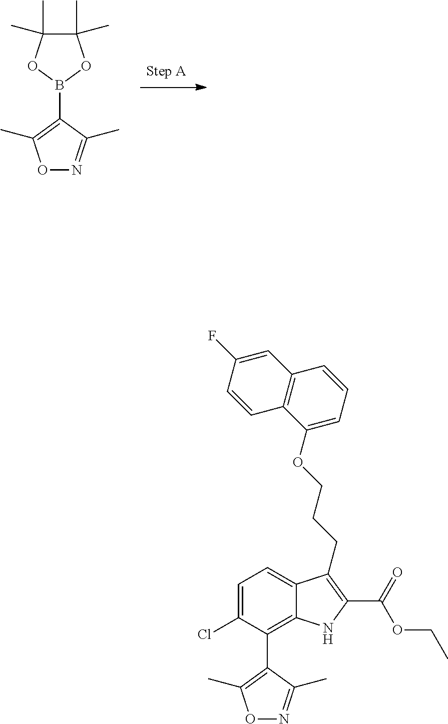

To a stirred solution of ethyl 7-bromo-6-chloro-3-(3-((6-fluoronaphthalen-1-yl)oxy)propyl)-1H-indole-2-carboxylate (250 mg, 0.495 mmol) in dioxane (3.6 mL) and water (0.9 mL), were added 3,5-dimethyl-4-(4,4,5,5-tetramethyl-1,3,2-dioxaborolan-2-yl)isoxazole (220 mg, 0.986 mmol) and K₃PO₄ (1.325 g, 1.482 mmol) at room temperature under nitrogen. The mixture was deoxygenated with argon and to it was added PdCl₂(dtbpf) (65 mg, 0.099 mmol) under argon atmosphere. Then the reaction mixture was heated at 100° C. for 16 h. After complete consumption of the starting material (monitored by TLC and LCMS), the reaction mixture was filtered through Celite® pad and the filtrate was evaporated under reduced pressure. It was then diluted with EtOAc and washed successively with water and brine. The organic layer was dried over Na₂SO₄ and evaporated under reduced pressure to get the crude compound, which was then purified by column chromatography (SiO₂, 30-50% EtOAc in DCM) to get of ethyl 6-chloro-7-(3,5-dimethylisoxazol-4-yl)-3-(3-((6-fluoronaphthalen-1-yl)oxy)propyl)-1H-indole-2-carboxylate (180 mg, 0.345 mmol, 70%) as brown solid.

LCMS (ESI+): m/z 521.0 [M+H]⁺

### Step B

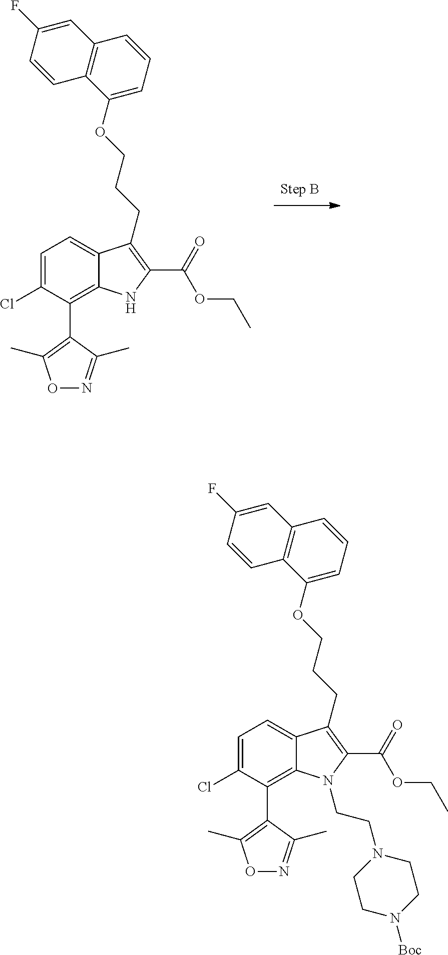 [SMILES: CCOC(=O)c1[nH]c2c(-c3c(C)noc3C)c(Cl)ccc2c1CCCOc1cccc2cc(F)ccc12 ; CCOC(=O)c1c(CCCOc2cccc3cc(F)ccc23)c2ccc(Cl)c(-c3c(C)noc3C)c2n1CCN1CCN(C(=O)OC(C)(C)C)CC1]

To a well stirred solution of ethyl 6-chloro-7-(3,5-dimethylisoxazol-4-yl)-3-(3-((6-fluoronaphthalen-1-yl)oxy)propyl)-1H-indole-2-carboxylate (180 mg, 0.345 mmol) in DMF (2 mL) was added tert-butyl 4-(2-chloroethyl)piperazine-1-carboxylate (130 mg, 0.52 mmol) followed by Cs₂CO₃ (169 mg, 0.519 mmol) at room temperature under nitrogen. The resulting mixture was allowed to stir at 90° C. for 16 h under nitrogen. After complete consumption of the starting material (monitored by TLC and LCMS), the reaction mixture was diluted with EtOAc and washed successively with water and brine. The organic layer was dried over Na₂SO₄ and evaporated under reduced pressure to get crude compound which was then purified by column chromatography (SiO₂, 50-60% EtOAc in DCM) to get ethyl 1-(2-(4-(tert-butoxycarbonyl)piperazin-1-yl)ethyl)-6-chloro-7-(3,5-dimethylisoxazol-4-yl)-3-(3-((6-fluoronaphthalen-1-yl)oxy)propyl)-1H-indole-2-carboxylate (150 mg, 0.205 mmol, 59%) as light brown solid.

LCMS (ESI+): m/z 733.0 [M+H]⁺

### Step C

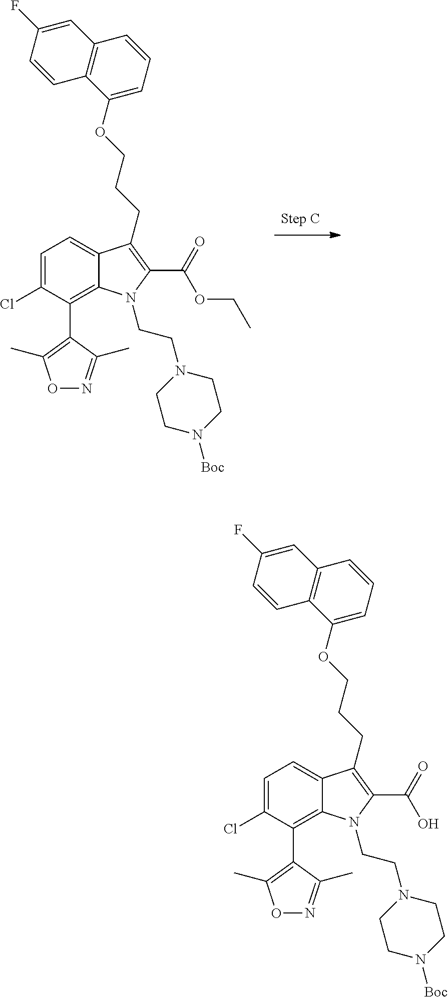 [SMILES: CCOC(=O)c1c(CCCOc2cccc3cc(F)ccc23)c2ccc(Cl)c(-c3c(C)noc3C)c2n1CCN1CCN(C(=O)OC(C)(C)C)CC1 ; Cc1noc(C)c1-c1c(Cl)ccc2c(CCCOc3cccc4cc(F)ccc34)c(C(=O)O)n(CCN3CCN(C(=O)OC(C)(C)C)CC3)c12]

Ethyl 1-(2-(4-(tert-butoxycarbonyl)piperazin-1-yl)ethyl)-6-chloro-7-(3,5-dimethylisoxazol-4-yl)-3-(3-((6-fluoronaphthalen-1-yl)oxy)propyl)-1H-indole-2-carboxylate (150 mg, 0.205 mmol) was dissolved in EtOH (4 mL) and solution of NaOH (33 mg, 0.82 mmol) in water (1 mL) was added to it. The mixture was heated under reflux for 16 h. After complete consumption of the starting material (monitored by TLC and LCMS), the reaction mixture was cooled down to room temperature and solvents were evaporated under reduced pressure. It was then diluted with EtOAc and washed with water. Aqueous layer was carefully acidified using 1M HCl to pH=3, extracted with EtOAc, dried over Na₂SO₄, filtered, and concentrated in vacuo. Crude 1-(2-(4-(tert-butoxycarbonyl)piperazin-1-yl)ethyl)-6-chloro-7-(3,5-dimethylisoxazol-4-yl)-3-(3-((6-fluoronaphthalen-1-yl)oxy)propyl)-1H-indole-2-carboxylic acid (100 mg) was obtained as brown gummy solid which was directly used in the next step without further purification.

LCMS (ESI+): m/z 705.4 [M+H]⁺

### Step D

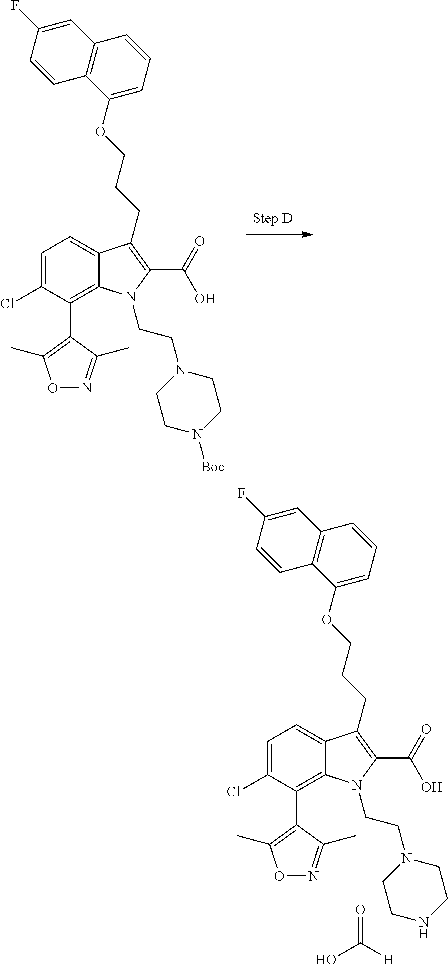

Crude 1-(2-(4-(tert-butoxycarbonyl)piperazin-1-yl)ethyl)-6-chloro-7-(3,5-dimethylisoxazo-4-yl)-3-(3-((6-fluoronaphthalen-1-yl)oxy)propyl)-1H-indole-2-carboxylic acid (100 mg) was dissolved in 4M HCl in dioxane (2 mL) at 0° C. and the mixture stirred for 2 h under nitrogen at room temperature. Where upon LCMS indicated the reaction was complete, the volatiles were concentrated in vacuum to get the crude compound which was then purified by reverse phase preparative HPLC (C18, H₂O:MeCN+0.1% FA) to get 4-{2-[2-carboxy-6-chloro-7-(3,5-dimethyl-1,2-oxazol-4-yl)-3-{3-[(6-fluoronaphthalen-1-yl)oxy]propyl}-1H-indol-1-yl]ethyl}piperazin-1-ium formate (22 mg, 0.036 mmol, 18% over 2 steps). Product was obtained as white solid.

LCMS (ESI+): m/z 605.4 [M+H]⁺

### Step E

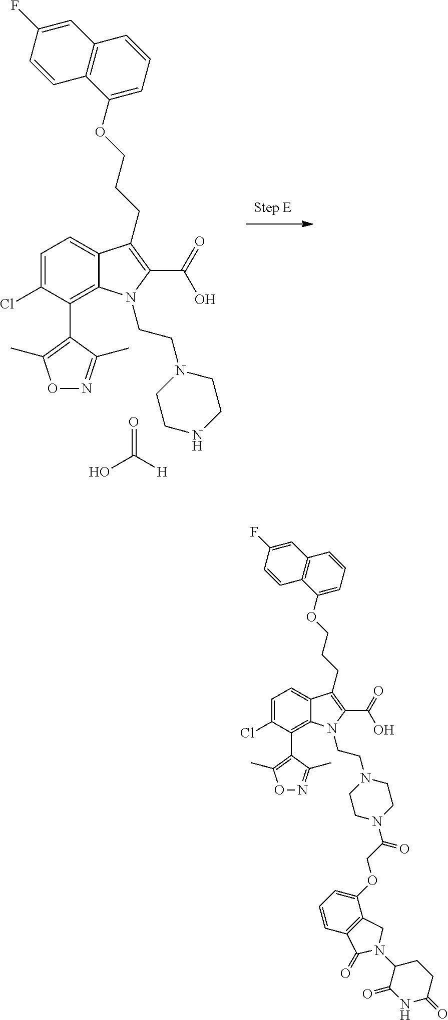

2-{[2-(2,6-Dioxopiperidin-3-yl)-1-oxo-2,3-dihydro-1H-isoindol-4-yl]oxy}acetic acid (18.7 mg, 0.059 mmol) and HATU (20.1 mg, 0.053 mmol) were dissolved in dry DMF (3.7 mL) and DIPEA (0.015 mL, 0.088 mmol) was added. Reaction was stirred at room temperature for 1 h. 4-{2-[2-carboxy-6-chloro-7-(3,5-dimethyl-1,2-oxazol-4-yl)-3-{3-[(6-fluoronaphthalen-1-yl)oxy]propyl}-1H-indol-1-yl]ethyl}piperazin-1-ium formate (17.3 mg, 0.027 mmol) was dissolved in aqueous 1M HCl (2 mL) and evaporated to dryness. The procedure was repeated twice and the resulting residue with added DIPEA (0.015 mL, 0.088 mmol) were dissolved in DMSO (0.916 mL) and added dropwise to the reaction. Reaction (monitored with LCMS) was continued at room temperature for 20 min. After complete consumption of the starting material, the mixture was concentrated under reduced pressure and diluted with DMSO and the product was purified with preparative HPLC (C18, H₂O:MeCN+0.1% FA) to get 6-chloro-7-(3,5-dimethylisoxazol-4-yl)-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-1H-indole-2-carboxylic acid (5.9 mg, 0.007 mmol, 22%) as white solid.

LCMS (ESI+): m/z 905.15 [M+H]⁺

¹H NMR (500 MHz, DMSO, 353 K) δ 10.64 (s, 1H), 8.23 (dd, J=9.2, 5.9 Hz, 1H), 7.81 (d, J=8.6 Hz, 1H), 7.58 (dd, J=10.4, 2.6 Hz, 1H), 7.49-7.37 (m, 3H), 7.36-7.30 (m, 2H), 7.28 (d, J=8.5 Hz, 1H), 7.14 (d, J=8.1 Hz, 1H), 6.87 (dd, J=5.7, 2.9 Hz, 1H), 5.04 (dd, J=13.0, 5.2 Hz, 1H), 4.89 (s, 2H), 4.40 (d, J=17.1 Hz, 1H), 4.37-4.28 (m, 2H), 4.25 (t, J=6.2 Hz, 2H), 4.22-4.14 (m, 1H), 3.41-3.33 (m, 4H), 3.32-3.26 (m, 2H), 2.88 (ddd, J=17.3, 13.4, 5.5 Hz, 1H), 2.66-2.58 (m, 1H), 2.48-2.40 (m, 2H), 2.28-2.21 (m, 5H), 2.21-2.13 (m, 6H), 2.08-2.02 (m, 1H), 2.01 (s, 3H).

### Example 140. 6-Chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(pyrazolo[1,5-a]pyrimidin-3-yl)-1H-indole-2-carboxylic acid (348)

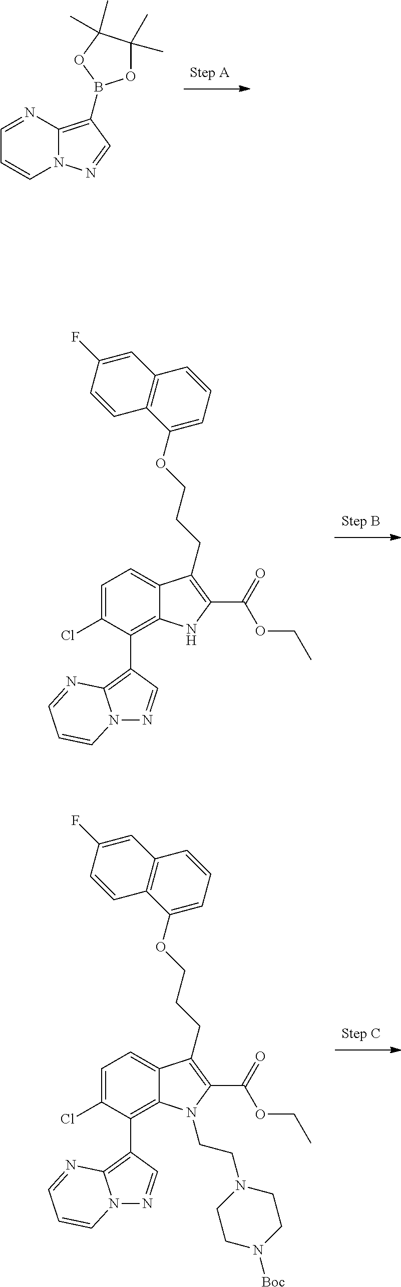 [SMILES: CC1(C)OB(c2cnn3cccnc23)OC1(C)C ; CCOC(=O)c1[nH]c2c(-c3cnn4cccnc34)c(Cl)ccc2c1CCCOc1cccc2cc(F)ccc12 ; CCOC(=O)c1c(CCCOc2cccc3cc(F)ccc23)c2ccc(Cl)c(-c3cnn4cccnc34)c2n1CCN1CCN(C(=O)OC(C)(C)C)CC1]

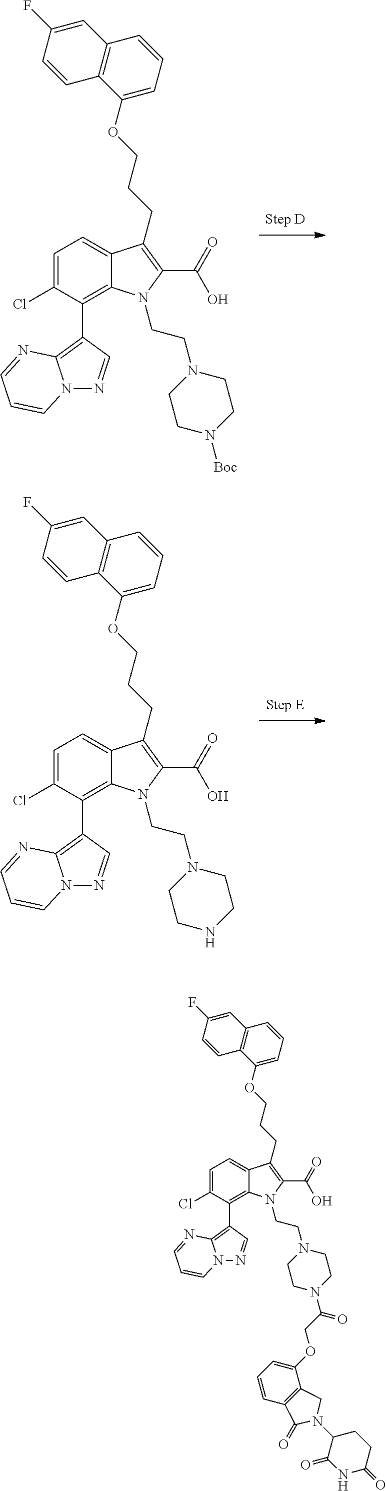 [SMILES: CC(C)(C)OC(=O)N1CCN(CCn2c(C(=O)O)c(CCCOc3cccc4cc(F)ccc34)c3ccc(Cl)c(-c4cnn5cccnc45)c32)CC1 ; O=C(O)c1c(CCCOc2cccc3cc(F)ccc23)c2ccc(Cl)c(-c3cnn4cccnc34)c2n1CCN1CCNCC1 ; O=C1CCC(N2Cc3c(OCC(=O)N4CCN(CCn5c(C(=O)O)c(CCCOc6cccc7cc(F)ccc67)c6ccc(Cl)c(-c7cnn8cccnc78)c65)CC4)cccc3C2=O)C(=O)N1]

### Step A

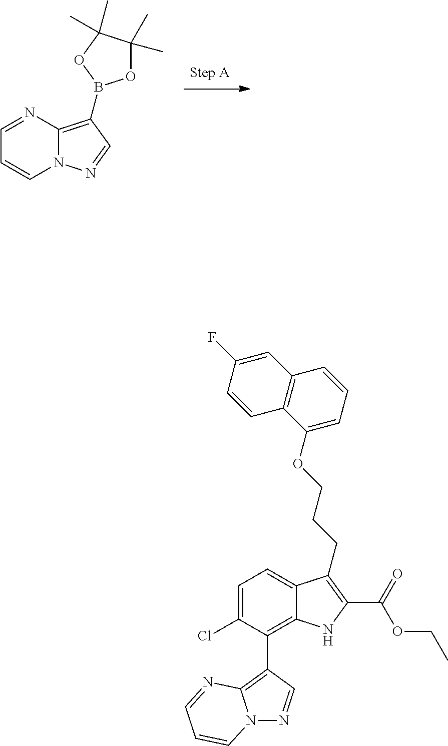 [SMILES: CC1(C)OB(c2cnn3cccnc23)OC1(C)C ; CCOC(=O)c1[nH]c2c(-c3cnn4cccnc34)c(Cl)ccc2c1CCCOc1cccc2cc(F)ccc12]

To a stirred solution of ethyl 7-bromo-6-chloro-3-(3-((6-fluoronaphthalen-1-yl)oxy)propyl)-1H-indole-2-carboxylate (300 mg, 0.594 mmol) in dioxane (3.6 mL) and water (0.9 mL), were added 3-(4,4,5,5-tetramethyl-1,3,2-dioxaborolan-2-yl)pyrazolo[1,5-a]pyrimidine (218 mg, 0.90 mmol) and K₃PO₄ (378 mg, 1.78 mmol) at room temperature under nitrogen. The mixture was deoxygenated with argon and to it was added PdCl₂(dtbpf) (77 mg, 0.118 mmol) under argon atmosphere. Then the reaction mixture was heated under reflux for 16 h. After complete consumption of the starting material (monitored by TLC and LCMS), the reaction mixture was filtered through Celite® pad and evaporated under reduced pressure. It was then diluted with EtOAc, washed successively with water and brine. The organic layer was dried over Na₂SO₄ and evaporated under reduced pressure to get the crude compound, which was then purified by column chromatography (SiO₂, 30-50% EtOAc in DCM) to get ethyl 6-chloro-3-(3-((6-fluoronaphthalen-1-yl)oxy)propyl)-7-(pyrazolo[1,5-a]pyrimidin-3-yl)-1H-indole-2-carboxylate (250 mg, 0.46 mmol, 77%) as brown solid.

LCMS (ESI+): m/z 543.2 [M+H]⁺

### Step B

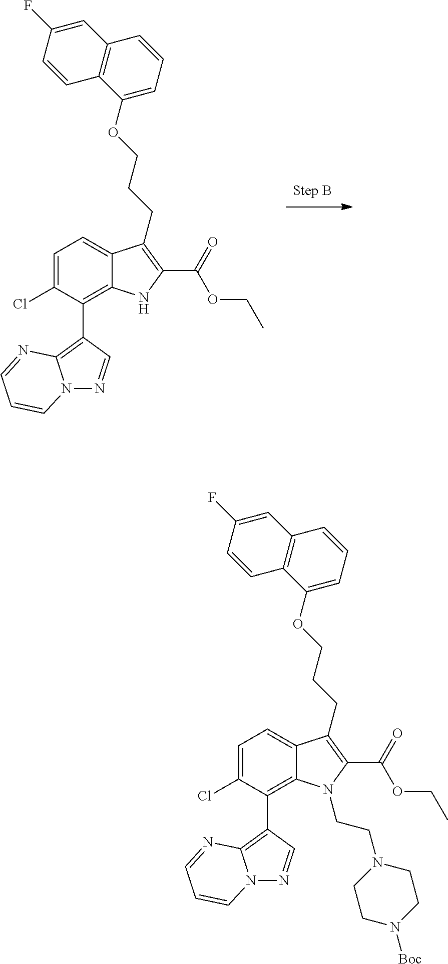 [SMILES: CCOC(=O)c1[nH]c2c(-c3cnn4cccnc34)c(Cl)ccc2c1CCCOc1cccc2cc(F)ccc12 ; CCOC(=O)c1c(CCCOc2cccc3cc(F)ccc23)c2ccc(Cl)c(-c3cnn4cccnc34)c2n1CCN1CCN(C(=O)OC(C)(C)C)CC1]

To a well stirred solution of ethyl 6-chloro-3-(3-((6-fluoronaphthalen-1-yl)oxy)propyl)-7-(pyrazolo[1,5-a]pyrimidin-3-yl)-1H-indole-2-carboxylate (250 mg, 0.46 mmol) in DMF (2 mL) was added tert-butyl 4-(2-chloroethyl)piperazine-1-carboxylate (103 mg, 0.414 mmol) followed by Cs₂CO₃ (225 mg, 0.69 mmol) at room temperature under nitrogen. The resulting mixture was allowed to stir at 90° C. for 16 h under nitrogen. After complete consumption of the starting material (monitored by TLC and LCMS), the reaction mixture was diluted with EtOAc, washed successively with water and brine, the organic layer was dried over Na₂SO₄ and evaporated under reduced pressure. Crude compound was purified by column chromatography (SiO₂, 2% MeOH in DCM) to get ethyl 1-(2-(4-(tert-butoxycarbonyl)piperazin-1-yl)ethyl)-6-chloro-3-(3-((6-fluoronaphthalen-1-yl)oxy)propyl)-7-(pyrazolo[1,5-a]pyrimidin-3-yl)-1H-indole-2-carboxylate (200 mg, 0.265 mmol, 57%) as brown solid.

LCMS (ESI+): m/z 755.5 [M+H]⁺

### Step C

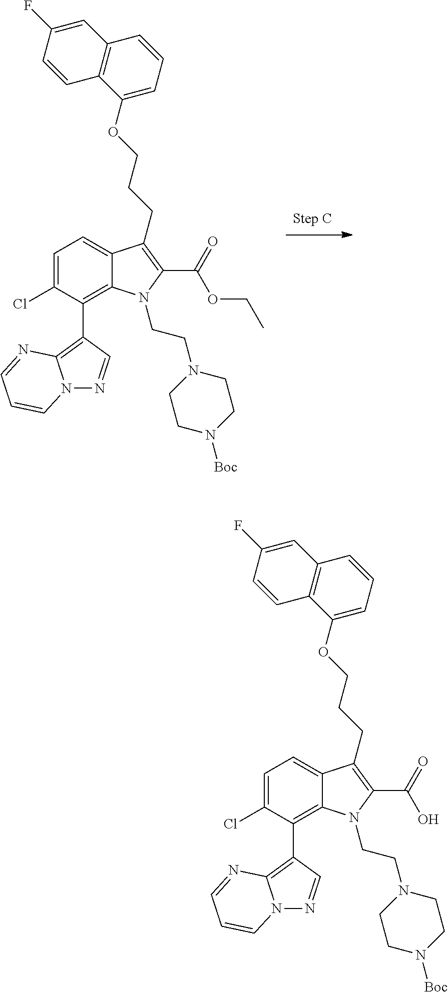 [SMILES: CCOC(=O)c1c(CCCOc2cccc3cc(F)ccc23)c2ccc(Cl)c(-c3cnn4cccnc34)c2n1CCN1CCN(C(=O)OC(C)(C)C)CC1 ; CC(C)(C)OC(=O)N1CCN(CCn2c(C(=O)O)c(CCCOc3cccc4cc(F)ccc34)c3ccc(Cl)c(-c4cnn5cccnc45)c32)CC1]

Ethyl 1-(2-(4-(tert-butoxycarbonyl)piperazin-1-yl)ethyl)-6-chloro-3-(3-((6-fluoronaphthalen-1-yl)oxy)propyl)-7-(pyrazolo[1,5-a]pyrimidin-3-yl)-1H-indole-2-carboxylate (200 mg, 0.265 mmol) was dissolved in EtOH (4 mL) and a solution of NaOH (44 mg, 1.1 mmol) in water (1 mL) was added to it. The mixture was heated under reflux for 16 h. After complete consumption of the starting material (monitored by TLC and LCMS), the reaction mixture was cooled down to room temperature and solvents were evaporated under reduced pressure. It was then diluted with EtOAc and washed with water. Aqueous layer was carefully acidified using aqueous 1M HCl to pH=3, extracted with EtOAc, dried over Na₂SO₄, filtered and concentrated in vacuo to afford 150 mg of crude 1-(2-(4-(tert-butoxycarbonyl)piperazin-1-yl)ethyl)-6-chloro-3-(3-((6-fluoronaphthalen-1-yl)oxy)propyl)-7-(pyrazolo[1,5-a]pyrimidin-3-yl)-1H-indole-2-carboxylic acid as dark brown gummy solid which was directly used in the next step without further purification.

LCMS (ESI+): m/z 727.6 [M+H]⁺

### Step D

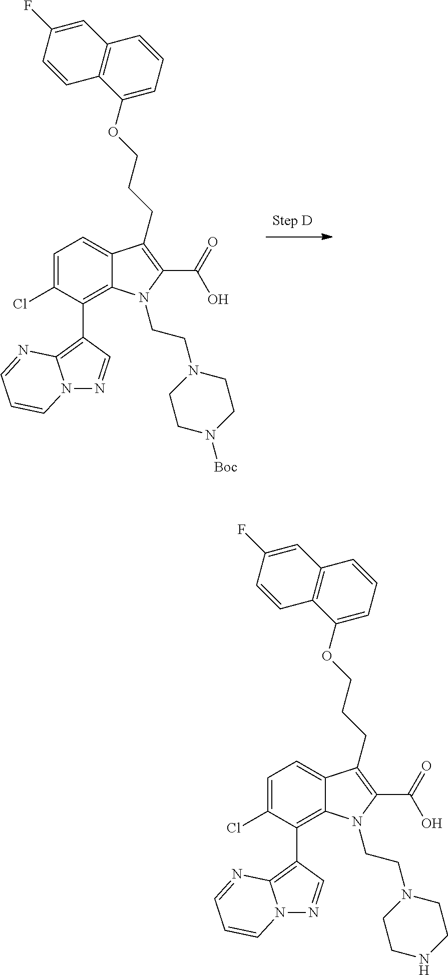 [SMILES: CC(C)(C)OC(=O)N1CCN(CCn2c(C(=O)O)c(CCCOc3cccc4cc(F)ccc34)c3ccc(Cl)c(-c4cnn5cccnc45)c32)CC1 ; O=C(O)c1c(CCCOc2cccc3cc(F)ccc23)c2ccc(Cl)c(-c3cnn4cccnc34)c2n1CCN1CCNCC1]

Crude 1-(2-(4-(tert-butoxycarbonyl)piperazin-1-yl)ethyl)-6-chloro-3-(3-((6-fluoronaphthalen-1-yl)oxy)propyl)-7-(pyrazolo[1,5-a]pyrimidin-3-yl)-1H-indole-2-carboxylic acid (150 mg) was dissolved in 4M HCl in dioxane (2 mL) at 0° C. and the mixture was stirred for 2 h under nitrogen at room temperature. Where upon LCMS indicated the reaction was complete, the volatiles were concentrated in vacuum to get the crude compound which was then purified by reverse phase preparative HPLC (C18, H₂O+20 mM ammonium bicarbonate:MeCN) to get 6-chloro-3-(3-((6-fluoronaphthalen-1-yl)oxy)propyl)-1-(2-(piperazin-1-yl)ethyl)-7-(pyrazolo[1,5-a]pyrimidin-3-yl)-1H-indole-2-carboxylic acid (65 mg, 0.103 mmol, 39% over 2 steps) as white solid.

LCMS (ESI+): m/z 627.4 [M+H]⁺

### Step E

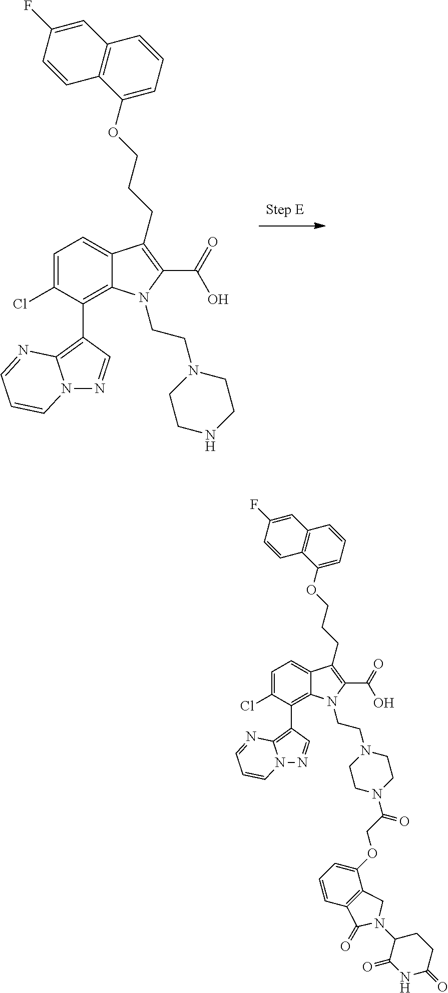 [SMILES: O=C(O)c1c(CCCOc2cccc3cc(F)ccc23)c2ccc(Cl)c(-c3cnn4cccnc34)c2n1CCN1CCNCC1 ; O=C1CCC(N2Cc3c(OCC(=O)N4CCN(CCn5c(C(=O)O)c(CCCOc6cccc7cc(F)ccc67)c6ccc(Cl)c(-c7cnn8cccnc78)c65)CC4)cccc3C2=O)C(=O)N1]

2-{[2-(2,6-Dioxopiperidin-3-yl)-1-oxo-2,3-dihydro-1H-isoindol-4-yl]oxy}acetic acid (13.7 mg, 0.043 mmol) and HATU (14.6 mg, 0.038 mmol) were dissolved in dry DMF (3.0 mL) under argon atmosphere and DIPEA (0.012 mL, 0.072 mmol) was added. The solution was stirred for 1 h at room temperature. To it, a suspension of 6-chloro-3-{3-[(6-fluoronaphthalen-1-yl)oxy]propyl}-1-[2-(piperazin-1-yl)ethyl]-7-{pyrazolo[1,5-a]pyrimidin-3-yl}-1H-indole-2-carboxylic acid (15.0 mg, 0.024 mmol) with DIPEA (0.012 mL, 0.072 mmol) in dry DMSO (0.747 mL) was added and the reaction (monitored with LCMS) was stirred for 30 min at room temperature under argon atmosphere. After complete conversion, crude product was directly purified with preparative HPLC (C18, H₂O:MeCN+0.1% FA). 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(pyrazolo[1,5-a]pyrimidin-3-yl)-1H-indole-2-carboxylic acid (15.9 mg, 0.017 mmol, 71.5%) was obtained as white solid.

LCMS (ESI+): m/z 927.15 [M+H]⁺

¹H NMR (500 MHz, DMSO, 353 K) δ 10.64 (s, 1H), 9.16 (dt, J=7.0, 1.6 Hz, 1H), 8.55 (dd, J=4.0, 1.7 Hz, 1H), 8.32 (s, 1H), 8.21 (dd, J=9.2, 5.9 Hz, 1H), 7.79 (d, J=8.5 Hz, 1H), 7.59 (dd, J=10.4, 2.6 Hz, 1H), 7.47-7.40 (m, 3H), 7.39-7.30 (m, 2H), 7.25 (d, J=8.5 Hz, 1H), 7.15-7.11 (m, 1H), 7.10 (dd, J=7.0, 4.0 Hz, 1H), 6.89 (p, J=4.7 Hz, 1H), 5.04 (dd, J=13.0, 5.2 Hz, 1H), 4.85 (s, 2H), 4.39 (d, J=17.1 Hz, 1H), 4.31 (d, J=17.1 Hz, 1H), 4.26 (t, J=6.2 Hz, 2H), 4.16-4.03 (m, 2H), 3.34-3.30 (m, 2H), 3.26-3.20 (m, 4H), 2.88 (ddd, J=17.3, 13.4, 5.5 Hz, 1H), 2.67-2.60 (m, 1H), 2.49-2.40 (m, 1H), 2.29-2.22 (m, 2H), 2.12-1.98 (m, 3H), 1.98-1.87 (m, 4H).

### Example 141. 6-Chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(5-(methoxymethyl)-1,3-dimethyl-1H-pyrazol-4-yl)-1H-indole-2-carboxylic acid (349)

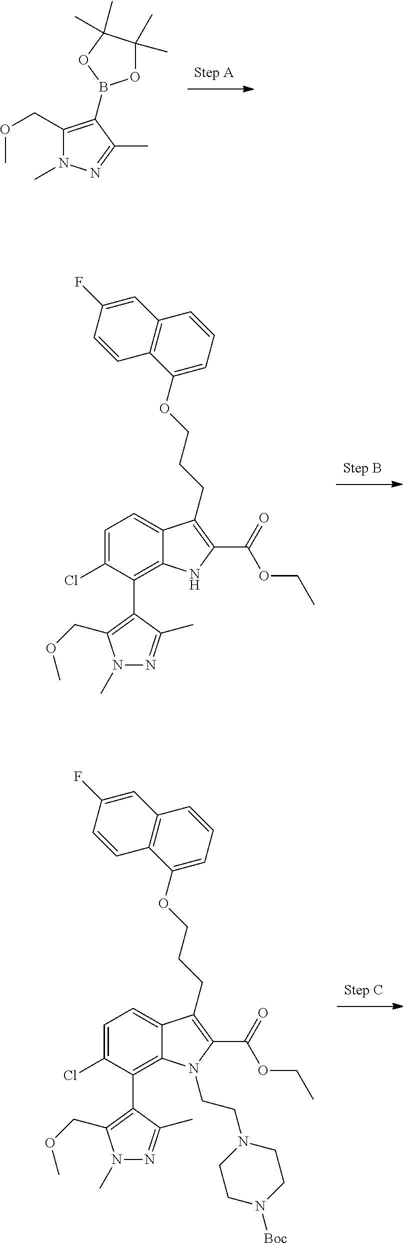 [SMILES: COCc1c(B2OC(C)(C)C(C)(C)O2)c(C)nn1C ; CCOC(=O)c1[nH]c2c(-c3c(C)nn(C)c3COC)c(Cl)ccc2c1CCCOc1cccc2cc(F)ccc12 ; CCOC(=O)c1c(CCCOc2cccc3cc(F)ccc23)c2ccc(Cl)c(-c3c(C)nn(C)c3COC)c2n1CCN1CCN(C(=O)OC(C)(C)C)CC1]

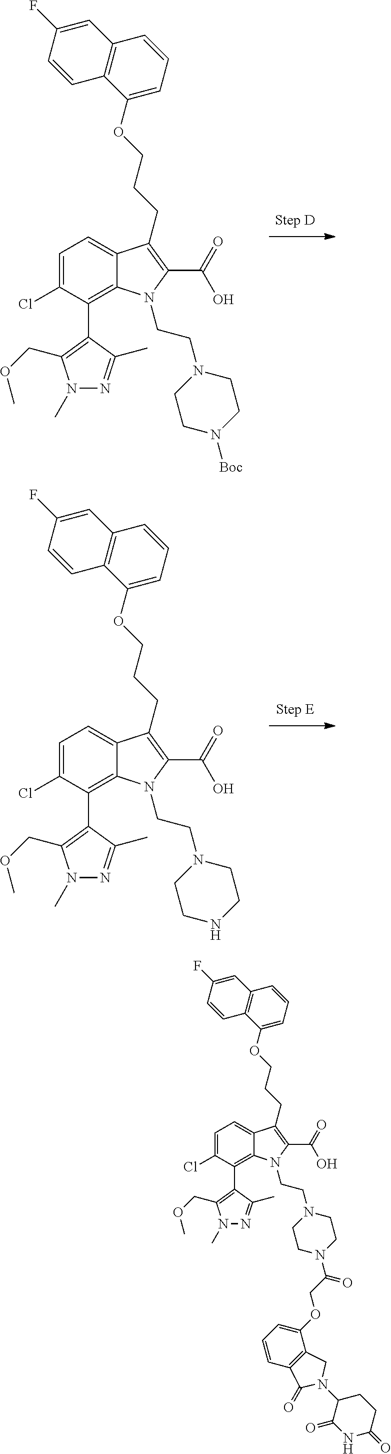

### Step A

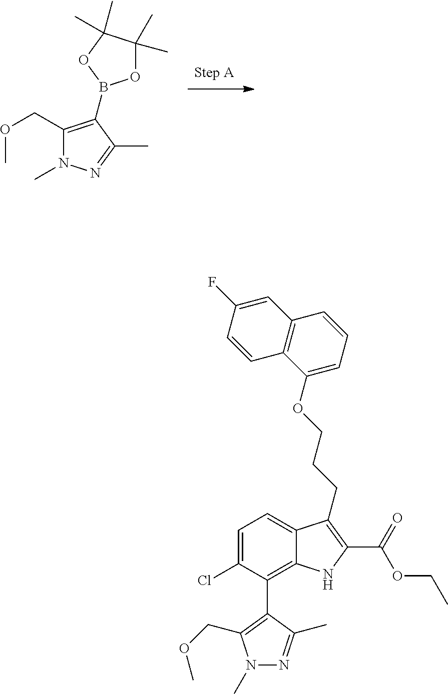 [SMILES: COCc1c(B2OC(C)(C)C(C)(C)O2)c(C)nn1C ; CCOC(=O)c1[nH]c2c(-c3c(C)nn(C)c3COC)c(Cl)ccc2c1CCCOc1cccc2cc(F)ccc12]

To a stirred solution of ethyl 7-bromo-6-chloro-3-(3-((6-fluoronaphthalen-1-yl)oxy)propyl)-1H-indole-2-carboxylate (250 mg, 0.495 mmol) in dioxane (3.6 mL) and water (0.9 mL), were added 5-(methoxymethyl)-1,3-dimethyl-4-(4,4,5,5-tetramethyl-1,3,2-dioxaborolan-2-yl)-1H-pyrazole (263 mg, 0.99 mmol) and K₃PO₄ (314 mg, 1.48 mmol) at room temperature under nitrogen. The mixture was deoxygenated with argon and to it was added PdCl₂(dtbpf) (64 mg, 0.099 mmol) under argon atmosphere. Then the reaction mixture was heated under reflux for 16 h. After complete consumption of the starting material (monitored by TLC and LCMS), the reaction mixture was filtered through Celite® pad and the filtrate was evaporated under reduced pressure. It was then diluted with EtOAc and washed successively with water and brine. The organic layer was dried over Na₂SO₄ and evaporated under reduced pressure to get 220 mg crude of ethyl 6-chloro-3-(3-((6-fluoronaphthalen-1-yl)oxy)propyl)-7-(5-(methoxymethyl)-1,3-dimethyl-1H-pyrazol-4-yl)-1H-indole-2-carboxylate as brown solid which was then directly used in the next step without any purification.

LCMS (ESI+): m/z 564.3 [M+H]⁺

### Step B

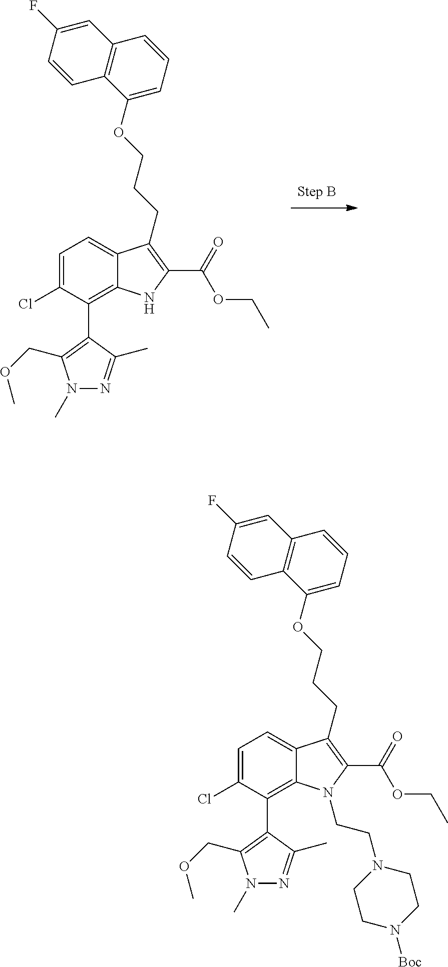 [SMILES: CCOC(=O)c1[nH]c2c(-c3c(C)nn(C)c3COC)c(Cl)ccc2c1CCCOc1cccc2cc(F)ccc12 ; CCOC(=O)c1c(CCCOc2cccc3cc(F)ccc23)c2ccc(Cl)c(-c3c(C)nn(C)c3COC)c2n1CCN1CCN(C(=O)OC(C)(C)C)CC1]

To a well stirred solution of ethyl 6-chloro-3-(3-((6-fluoronaphthalen-1-yl)oxy)propyl)-7-(5-(methoxymethyl)-1,3-dimethyl-1H-pyrazol-4-yl)-1H-indole-2-carboxylate (220 mg, crude) in DMF (2 mL) was added tert-butyl 4-(2-chloroethyl)piperazine-1-carboxylate (145 mg, 0.583 mmol) followed by Cs₂CO₃ (191 mg, 0.586 mmol) at room temperature under nitrogen. The resulting mixture was allowed to stir at 90° C. for 16 h under nitrogen. After complete consumption of the starting material (monitored by TLC and LCMS), the reaction mixture was diluted with EtOAc and washed successively with water and brine. The organic layer was dried over Na₂SO₄ and evaporated under reduced pressure to get 200 mg of crude ethyl 1-(2-(4-(tert-butoxycarbonyl)piperazin-1-yl)ethyl)-6-chloro-3-(3-((6-fluoronaphthalen-1-yl)oxy)propyl)-7-(5-(methoxymethyl)-1,3-dimethyl-1H-pyrazol-4-yl)-1H-indole-2-carboxylate as brown solid, which was then directly used in the next step without further purification.

LCMS (ESI+): m/z 776.4 [M+H]⁺

### Step C

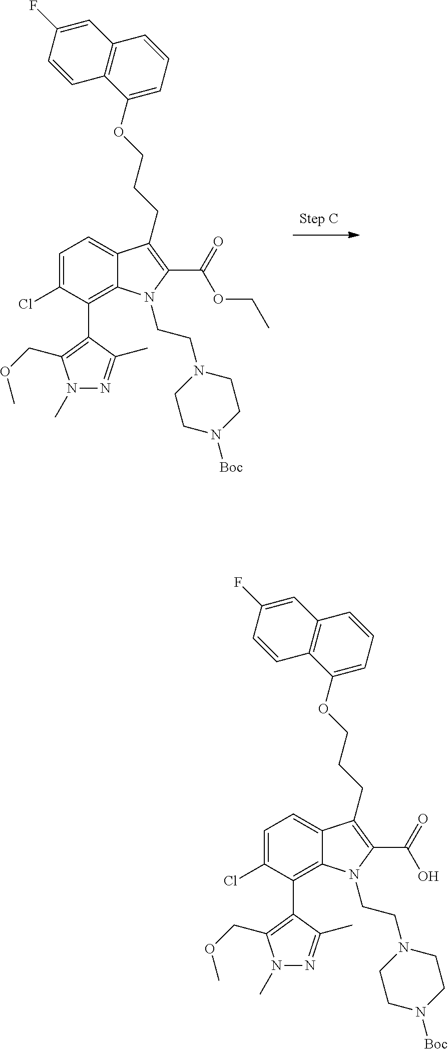 [SMILES: CCOC(=O)c1c(CCCOc2cccc3cc(F)ccc23)c2ccc(Cl)c(-c3c(C)nn(C)c3COC)c2n1CCN1CCN(C(=O)OC(C)(C)C)CC1 ; COCc1c(-c2c(Cl)ccc3c(CCCOc4cccc5cc(F)ccc45)c(C(=O)O)n(CCN4CCN(C(=O)OC(C)(C)C)CC4)c23)c(C)nn1C]

Ethyl 1-(2-(4-(tert-butoxycarbonyl)piperazin-1-yl)ethyl)-6-chloro-3-(3-((6-fluoronaphthalen-1-yl)oxy)propyl)-7-(5-(methoxymethyl)-1,3-dimethyl-1H-pyrazol-4-yl)-1H-indole-2-carboxylate (200 mg, crude) was dissolved in EtOH (4 mL) and solution of NaOH (41 mg, 1.03 mmol) in water (1 mL) was added to it. The mixture was heated under reflux for 16 h. After complete consumption of the starting material (monitored by TLC and LCMS), the reaction mixture was cooled down to room temperature, solvents were evaporated under reduced pressure. It was then diluted with EtOAc and washed with water. Aqueous layer was carefully acidified using 1M HCl to pH=3, extracted with EtOAc, dried over Na₂SO₄, filtered, and concentrated in vacuo. 1-(2-(4-(tert-butoxycarbonyl)piperazin-1-yl)ethyl)-6-chloro-3-(3-((6-fluoronaphthalen-1-yl)oxy)propyl)-7-(5-(methoxymethyl)-1,3-dimethyl-1H-pyrazol-4-yl)-1H-indole-2-carboxylic acid (150 mg, crude) was obtained as light brown gummy solid which was directly used in the next step without further purification.

LCMS (ESI+): m/z 748.2 [M+H]⁺

### Step D

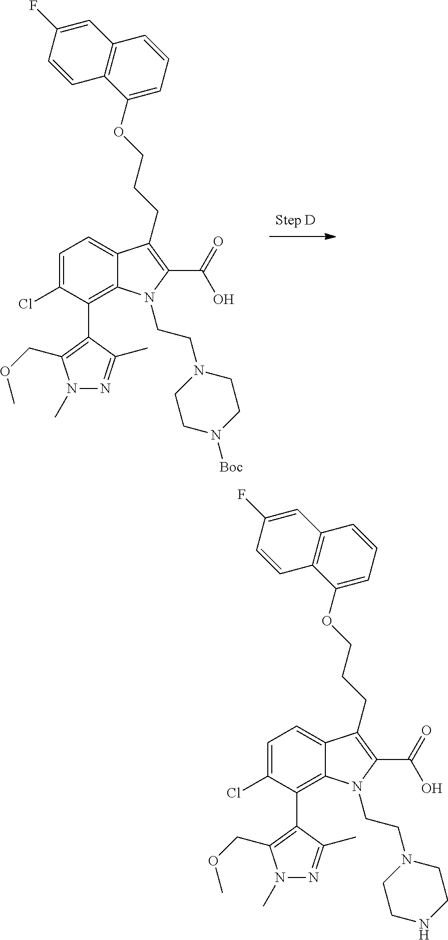

Crude 1-(2-(4-(tert-butoxycarbonyl)piperazin-1-yl)ethyl)-6-chloro-3-(3-((6-fluoronaphthalen-1-yl)oxy)propyl)-7-(5-(methoxymethyl)-1,3-dimethyl-1H-pyrazol-4-yl)-1H-indole-2-carboxylic acid (150 mg) was dissolved in 4M HCl in dioxane (2 mL) at 0° C. and the mixture stirred for 2 h under nitrogen at RT. Where upon LCMS indicated the reaction was complete, the volatiles were concentrated in vacuum to get the crude compound which was then purified by reverse phase preparative HPLC (C18, 20 mM ammonium bicarbonate in H₂O:MeCN) to get 6-chloro-3-(3-((6-fluoronaphthalen-1-yl)oxy)propyl)-7-(5-(methoxymethyl)-1,3-dimethyl-1H-pyrazol-4-yl)-1-(2-(piperazin-1-yl)ethyl)-1H-indole-2-carboxylic acid (50 mg, 0.077 mmol, 15% over 4 steps) as off white solid.

LCMS (ESI+): m/z 648.4 [M+H]⁺

### Step E

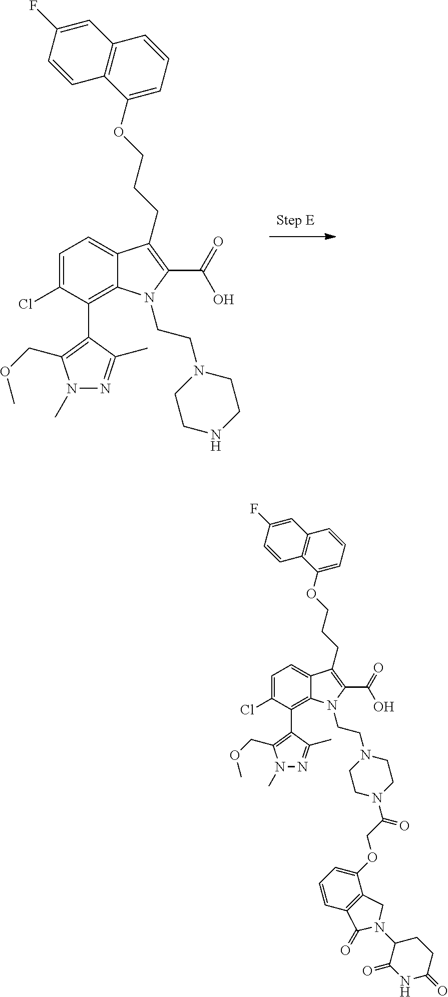 [SMILES: COCc1c(-c2c(Cl)ccc3c(CCCOc4cccc5cc(F)ccc45)c(C(=O)O)n(CCN4CCNCC4)c23)c(C)nn1C ; COCc1c(-c2c(Cl)ccc3c(CCCOc4cccc5cc(F)ccc45)c(C(=O)O)n(CCN4CCN(C(=O)COc5cccc6c5CN(C5CCC(=O)NC5=O)C6=O)CC4)c23)c(C)nn1C]

2-{[2-(2,6-dioxopiperidin-3-yl)-1-oxo-2,3-dihydro-1H-isoindol-4-yl]oxy}acetic acid (13.2 mg, 0.020 mmol) and HATU (13.9 mg, 0.037 mmol) were dissolved in dry DMF (2.5 mL) under argon atmosphere and DIPEA (0.011 mL, 0.061 mmol) was added. The solution was stirred for 1 h at room temperature. Next, the solution of 6-chloro-3-{3-[(6-fluoronaphthalen-1-yl)oxy]propyl}-7-[5-(methoxymethyl)-1,3-dimethyl-1H-pyrazol-4-yl]-1-[2-(piperazin-1-yl)ethyl]-1H-indole-2-carboxylic acid (13.2 mg, 0.020 mmol) and DIPEA (0.011 mL, 0.061 mmol) was slowly added dropwise and the reaction was stirred under argon atmosphere at room temperature for 30 min. LCMS indicated complete consumption of the starting material. The solution was diluted with DMSO and purified with preparative HPLC (C18, H₂O:MeCN+0.1% FA) to give 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(5-(methoxymethyl)-1,3-dimethyl-1H-pyrazol-4-yl)-1H-indole-2-carboxylic acid (12.4 mg, 0.013 mmol, 64.5%) as white solid.

LCMS (ESI+): m/z 948.2 [M+H]⁺

¹H NMR (500 MHz, DMSO, 353 K) δ 10.65 (s, 1H), 8.25 (dd, J=9.2, 5.9 Hz, 1H), 7.71 (d, J=8.5 Hz, 1H), 7.58 (dd, J=10.4, 2.6 Hz, 1H), 7.48-7.38 (m, 3H), 7.37-7.29 (m, 2H), 7.21 (d, J=8.5 Hz, 1H), 7.14 (d, J=8.1 Hz, 1H), 6.87 (dd, J=6.1, 2.6 Hz, 1H), 5.05 (dd, J=13.0, 5.2 Hz, 1H), 4.89 (s, 2H), 4.40 (d, J=17.1 Hz, 1H), 4.32 (d, J=17.1 Hz, 1H), 4.27-4.18 (m, 5H), 4.13 (d, J=12.4 Hz, 1H), 3.84 (d, J=1.5 Hz, 3H), 3.36 (t, J=5.1 Hz, 4H), 3.32-3.24 (m, 2H), 3.15 (s, 3H), 2.88 (ddd, J=17.4, 13.5, 5.5 Hz, 1H), 2.66-2.60 (m, 1H), 2.49-2.40 (m, 2H), 2.27-2.21 (m, 2H), 2.20-2.08 (m, 6H), 2.08-2.02 (m, 1H), 1.93 (s, 3H).

### Example 142. 6-Chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(pyrazolo[1,5-a]pyrazin-3-yl)-1H-indole-2-carboxylic acid (350)

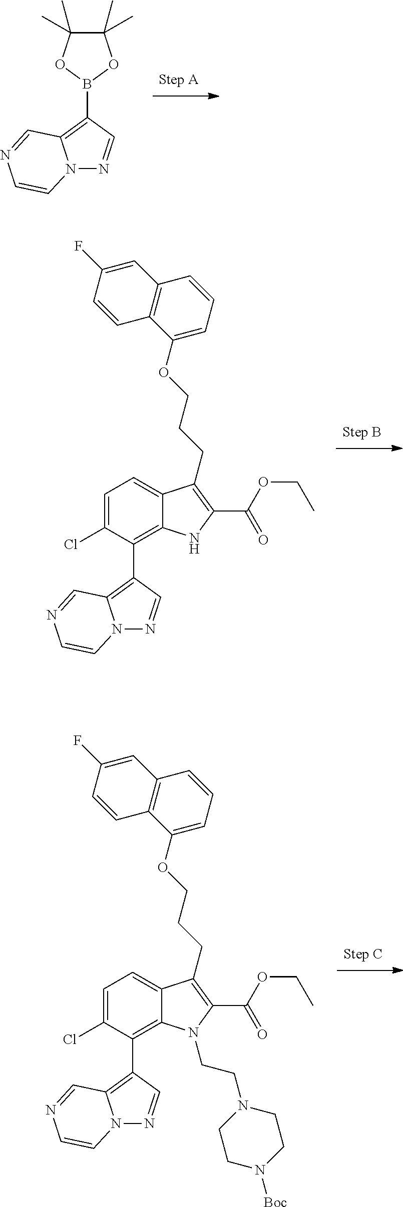 [SMILES: CC1(C)OB(c2cnn3ccncc23)OC1(C)C ; CCOC(=O)c1[nH]c2c(-c3cnn4ccncc34)c(Cl)ccc2c1CCCOc1cccc2cc(F)ccc12 ; CCOC(=O)c1c(CCCOc2cccc3cc(F)ccc23)c2ccc(Cl)c(-c3cnn4ccncc34)c2n1CCN1CCN(C(=O)OC(C)(C)C)CC1]

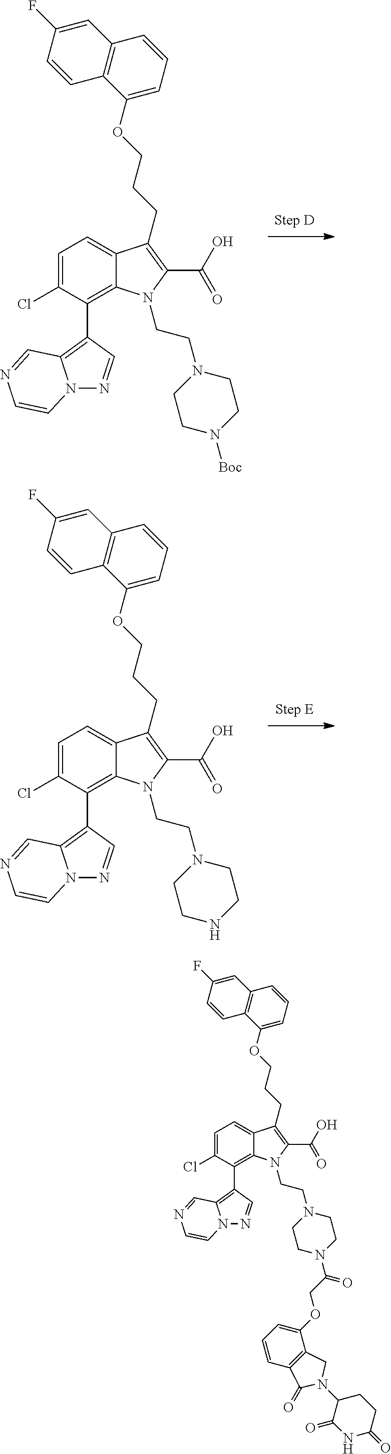

### Step A

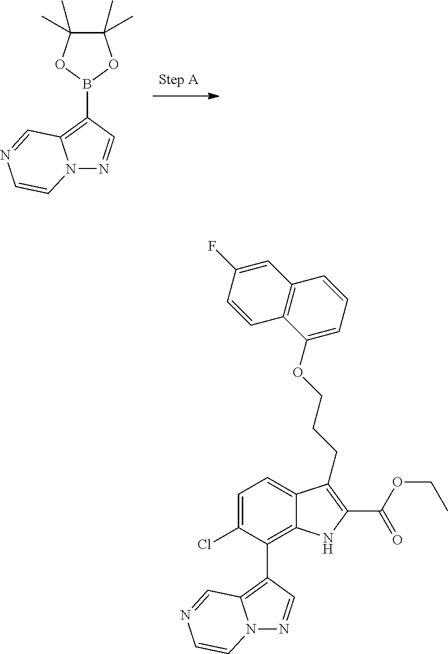 [SMILES: CC1(C)OB(c2cnn3ccncc23)OC1(C)C ; CCOC(=O)c1[nH]c2c(-c3cnn4ccncc34)c(Cl)ccc2c1CCCOc1cccc2cc(F)ccc12]

To a solution of ethyl 7-bromo-6-chloro-3-(3-((6-fluoronaphthalen-1-yl)oxy)propyl)-1H-indole-2-carboxylate (200 mg, 0.395 mmol) in dioxane (3.6 mL) and water (0.9 mL), were added 3-(4,4,5,5-tetramethyl-1,3,2-dioxaborolan-2-yl)pyrazolo[1,5-a]pyrazine (194 mg, 0.791 mmol) and K₃PO₄ (252 mg, 1.2 mmol) at room temperature under nitrogen. The mixture was deoxygenated with argon and to it was added PdCl₂(dtbpf) (51 mg, 0.079 mmol) under argon atmosphere. Then the reaction mixture was heated under reflux for 16 h. After complete consumption of the starting material (monitored by TLC and LCMS), the reaction mixture was filtered through Celite® pad and the filtrate was evaporated under reduced pressure. It was then diluted with EtOAc, washed successively with water and brine. The organic layer was dried over Na₂SO₄ and evaporated under reduced pressure to get crude ethyl 6-chloro-3-(3-((6-fluoronaphthalen-1-yl)oxy)propyl)-7-(pyrazolo[1,5-a]pyrazin-3-yl)-1H-indole-2-carboxylate (120 mg) as brown solid which was then directly used in the next step without any purification.

LCMS (ESI+): m/z 543.1 [M+H]⁺

### Step B

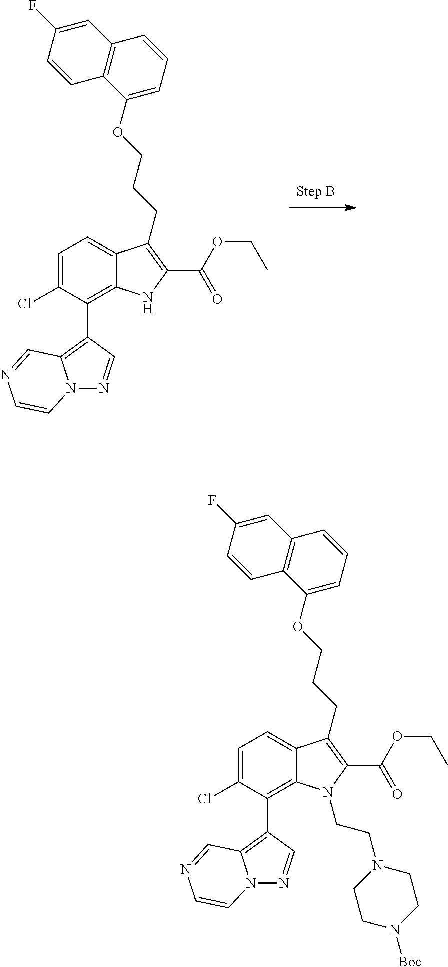 [SMILES: CCOC(=O)c1[nH]c2c(-c3cnn4ccncc34)c(Cl)ccc2c1CCCOc1cccc2cc(F)ccc12 ; CCOC(=O)c1c(CCCOc2cccc3cc(F)ccc23)c2ccc(Cl)c(-c3cnn4ccncc34)c2n1CCN1CCN(C(=O)OC(C)(C)C)CC1]

To a solution of crude ethyl 6-chloro-3-(3-((6-fluoronaphthalen-1-yl)oxy)propyl)-7-(pyrazolo[1,5-a]pyrazin-3-yl)-1H-indole-2-carboxylate (120 mg, crude) in DMF (2 mL) was added tert-butyl 4-(2-chloroethyl)piperazine-1-carboxylate (83 mg, 0.33 mmol) followed by Cs₂CO₃ (108 mg, 0.331 mmol) at room temperature under nitrogen. The resulting mixture was allowed to stir at 90° C. for 16 h under nitrogen. After complete consumption of the starting material (monitored by TLC and LCMS), the reaction mixture was diluted with EtOAc and washed successively with water and brine. The organic layer was dried over Na₂SO₄ and evaporated under reduced pressure to get crude compound which was purified with column chromatography (SiO₂, 30-40% EtOAc in DCM) to get ethyl 1-(2-(4-(tert-butoxycarbonyl)piperazin-1-yl)ethyl)-6-chloro-3-(3-((6-fluoronaphthalen-1-yl)oxy)propyl)-7-(pyrazolo[1,5-a]pyrazin-3-yl)-1H-indole-2-carboxylate (120 mg, 0.16 mmol, 40.5% over 2 steps) as light brown solid.

LCMS (ESI+): m/z 755.3 [M+H]⁺

### Step C

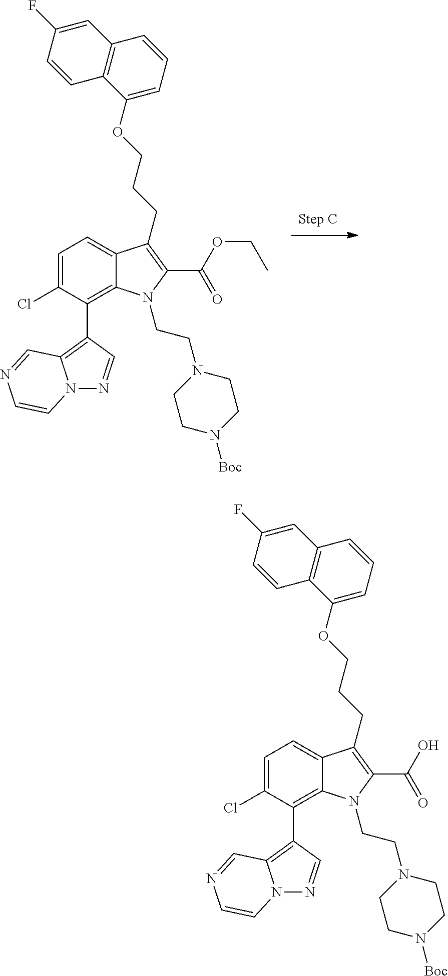

Ethyl 1-(2-(4-(tert-butoxycarbonyl)piperazin-1-yl)ethyl)-6-chloro-3-(3-((6-fluoronaphthalen-1-yl)oxy)propyl)-7-(pyrazolo[1,5-a]pyrazin-3-yl)-1H-indole-2-carboxylate (120 mg, 0.16 mmol) was dissolved in EtOH (4 mL) and solution of NaOH (41 mg, 1.03 mmol) in water (1 mL) was added to it. The mixture was heated under reflux for 16 h. After complete consumption of the starting material (monitored by TLC and LCMS), the reaction mixture was cooled down to room temperature, and solvents were evaporated under reduced pressure. The resulting residue was then diluted with water and washed with EtOAc. Aqueous layer was carefully acidified using 1 M HCl to pH=3, extracted with EtOAc, dried over Na₂SO₄, filtered, and concentrated in vacuo. Crude 1-(2-(4-(tert-butoxycarbonyl)piperazin-1-yl)ethyl)-6-chloro-3-(3-((6-fluoronaphthalen-1-yl)oxy)propyl)-7-(pyrazolo[1,5-a]pyrazin-3-yl)-1H-indole-2-carboxylic acid (80 mg) was obtained as brown gummy solid which was directly used in the next step without further purification.

LCMS (ESI+): m/z 727.4 [M+H]⁺.

### Step D

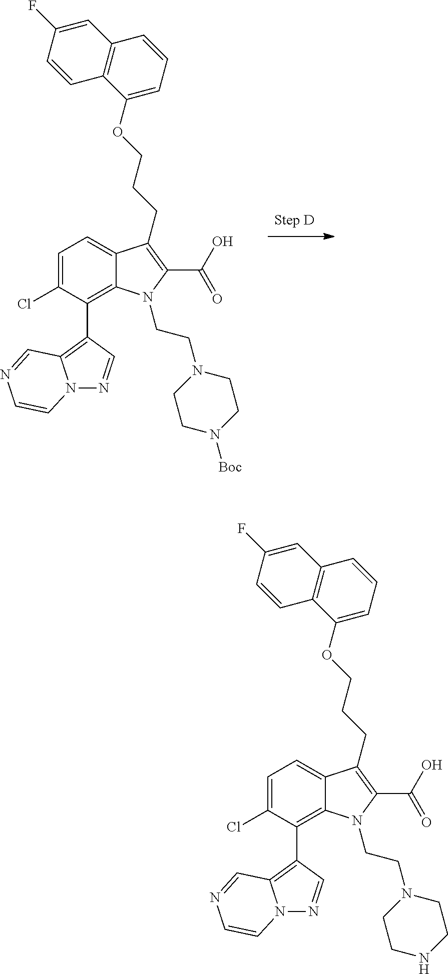 [SMILES: CC(C)(C)OC(=O)N1CCN(CCn2c(C(=O)O)c(CCCOc3cccc4cc(F)ccc34)c3ccc(Cl)c(-c4cnn5ccncc45)c32)CC1 ; O=C(O)c1c(CCCOc2cccc3cc(F)ccc23)c2ccc(Cl)c(-c3cnn4ccncc34)c2n1CCN1CCNCC1]

Crude 1-(2-(4-(tert-butoxycarbonyl)piperazin-1-yl)ethyl)-6-chloro-3-(3-((6-fluoronaphthalen-1-yl)oxy)propyl)-7-(pyrazolo[1,5-a]pyrazin-3-yl)-1H-indole-2-carboxylic acid (80 mg) was dissolved in 4M HCl in dioxane (2 mL) at 0° C. and the mixture was stirred for 2 h under nitrogen at room temperature. After complete consumption of the starting material (indicated by LCMS), the volatiles were concentrated under reduced pressure. Crude compound was purified by preparative HPLC (C18, 20 mM ammonium bicarbonate:MeCN) to get 6-chloro-3-(3-((6-fluoronaphthalen-1-yl)oxy)propyl)-1-(2-(piperazin-1-yl)ethyl)-7-(pyrazolo[1,5-a]pyrazin-3-yl)-1H-indole-2-carboxylic acid (10 mg, 0.016 mmol, 10% over 2 steps) as off white solid.

LCMS (ESI+): m/z 627.4 [M+H]⁺

### Step E

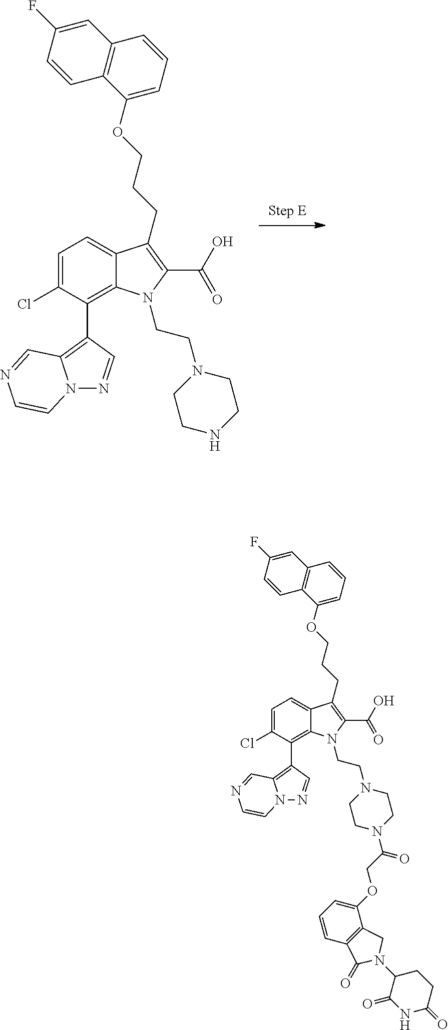 [SMILES: O=C(O)c1c(CCCOc2cccc3cc(F)ccc23)c2ccc(Cl)c(-c3cnn4ccncc34)c2n1CCN1CCNCC1 ; O=C1CCC(N2Cc3c(OCC(=O)N4CCN(CCn5c(C(=O)O)c(CCCOc6cccc7cc(F)ccc67)c6ccc(Cl)c(-c7cnn8ccncc78)c65)CC4)cccc3C2=O)C(=O)N1]

2-{[2-(2,6-Dioxopiperidin-3-yl)-1-oxo-2,3-dihydro-1H-isoindol-4-yl]oxy}acetic acid (7.4 mg, 0.023 mmol) was dissolved under argon atmosphere in dry DMF (1.6 mL) and DIPEA (0.007 mL, 0.039 mmol) followed by HATU (7.8 mg, 0.021 mmol) was added. The solution was stirred under argon for 1 h at room temperature. 6-chloro-3-{3-[(6-fluoronaphthalen-1-yl)oxy]propyl}-1-[2-(piperazin-1-yl)ethyl]-7-{pyrazolo[1,5-a]pyrazin-3-yl}-1H-indole-2-carboxylic acid (8.1 mg, 0.013 mmol) and DIPEA (0.007 mL, 0.039 mmol) were dissolved in dry DMSO (0.40 mL), added dropwise to the reaction and mixed at room temperature for the next 30 min. Then additional portion of HATU (7.8 mg, 0.021 mmol) with DIPEA (0.007 mL, 0.039 mmol) in DMF (1.0 mL) was added and the reaction was stirred for 4 h at 60° C. After complete consumption of the starting material (monitored with LCMS), the solution was concentrated under reduced pressure, diluted with DMSO and directly purified with preparative HPLC (C18, H₂O:MeCN+0.1% FA). 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(pyrazolo[1,5-a]pyrazin-3-yl)-1H-indole-2-carboxylic acid (4.4 mg, 0.005 mmol, 37%) was obtained as white solid.

LCMS (ESI+): m/z 927.1 [M+H]⁺

¹H NMR (500 MHz, DMSO, 353 K) δ 10.65 (s, 1H), 8.89-8.76 (m, 2H), 8.27 (s, 1H), 8.23 (dd, J=9.2, 5.8 Hz, 1H), 7.99 (d, J=4.8 Hz, 1H), 7.84 (d, J=8.6 Hz, 1H), 7.59 (dd, J=10.4, 2.7 Hz, 1H), 7.46-7.40 (m, 3H), 7.37-7.27 (m, 3H), 7.12 (d, J=8.1 Hz, 1H), 6.89 (p, J=4.7 Hz, 1H), 5.05 (dd, J=13.0, 5.2 Hz, 1H), 4.85 (s, 2H), 4.39 (d, J=17.1 Hz, 1H), 4.31 (d, J=17.1 Hz, 1H), 4.26 (t, J=6.2 Hz, 2H), 4.20-4.09 (m, 1H), 3.99-3.87 (m, 1H), 3.35-3.30 (m, 2H), 3.22 (t, J=5.1 Hz, 4H), 2.88 (ddd, J=18.2, 13.4, 5.5 Hz, 1H), 2.67-2.60 (m, 1H), 2.48-2.41 (m, 1H), 2.26 (p, J=6.9 Hz, 2H), 2.14-2.01 (m, 2H), 1.98-1.84 (m, 5H).

### Example 143: 1-{2-[4-(2-{[2-(2,6-Dioxopiperidin-3 yl)-oxo-2,3-dihydro-1H-isoindol-4-yl]oxy}acetyl)piperazin-1-yl]ethyl}-6-fluoro-3-{3-[(6-fluoronaphthalen-1-yl)oxy]propyl}-7-{6-methyl-2H,3H-pyrazolo[3,2-b][1,3]oxazol-7-yl}-1H-indole-2-carboxylic acid (351)

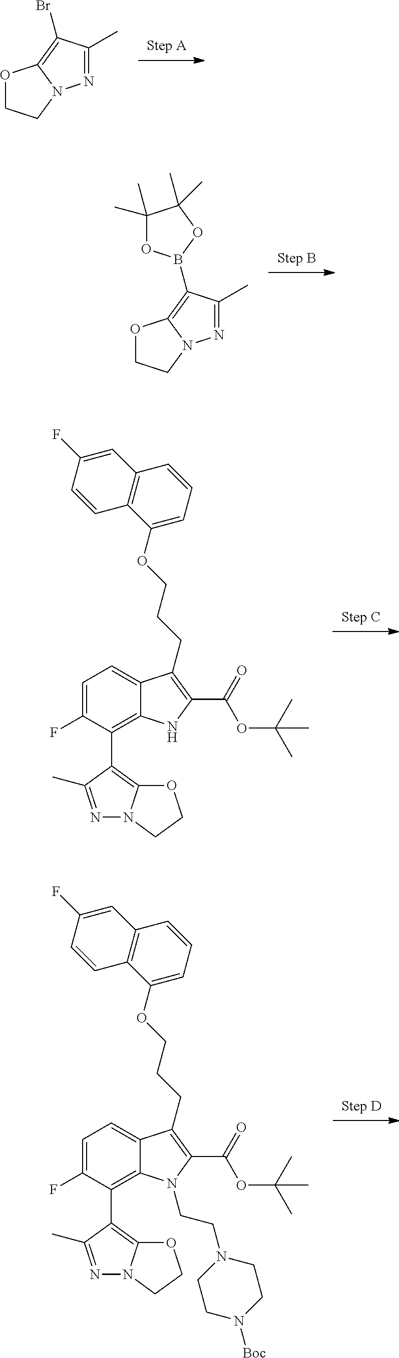

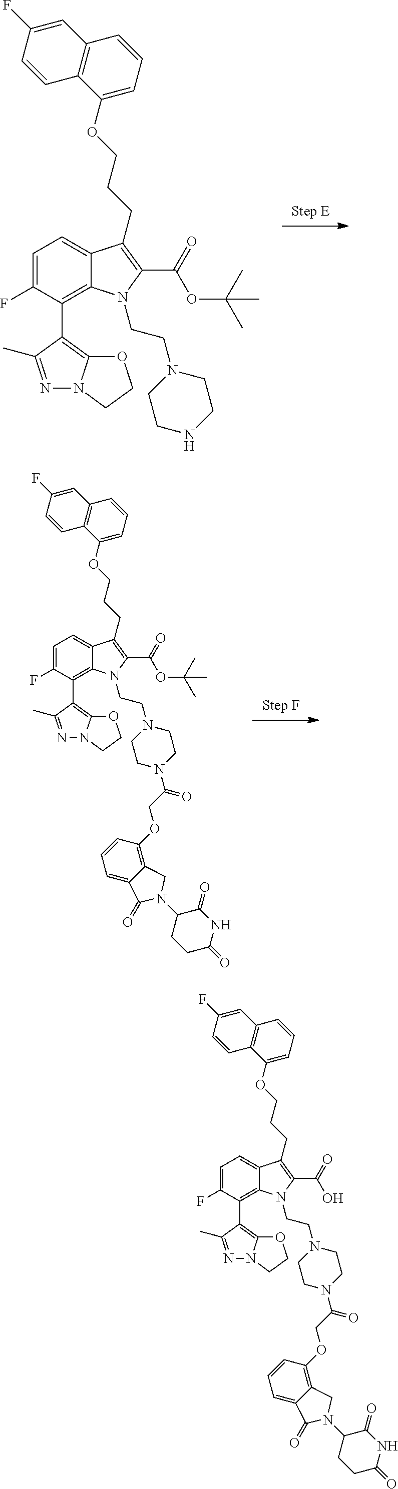

### Step A

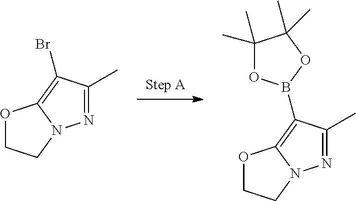

To a well stirred solution of 7-bromo-6-methyl-2,3-dihydropyrazolo[5,1-b]oxazole (2 g, 9.85 mmol) and 2-Isopropoxy-4,4,5,5-tetramethyl-1,3,2-dioxaborolane (10.04 mL, 49.26 mmol) in THE (60 mL), was added n-Butyllithium (1.8M, 16.4 mL, 29.55 mmol) at −78° C. under argon. The resulting mixture was then allowed to stir at −78° C. for 2 h. The mixture was slowly warmed to room temperature and was stirred for another 30 minutes. After complete consumption of the starting material the excess butyllithium was quenched by addition of saturated ammonium chloride and it was diluted with EtOAc and washed successively with water and brine. The organic layer was dried over Na₂SO₄ and evaporated under reduced pressure to get the crude compound, which was then purified using flash chromatography (SiO₂, 50-60% EtOAc in hexane) to get (750 mg, 3 mmol, 30%) of 6-methyl-7-(4,4,5,5-tetramethyl-1,3,2-dioxaborolan-2-yl)-2,3-dihydropyrazolo[5,1-b]oxazole as white sticky solid.

LCMS (ESI+): m/z 250.0 [M+H]⁺

### Step B

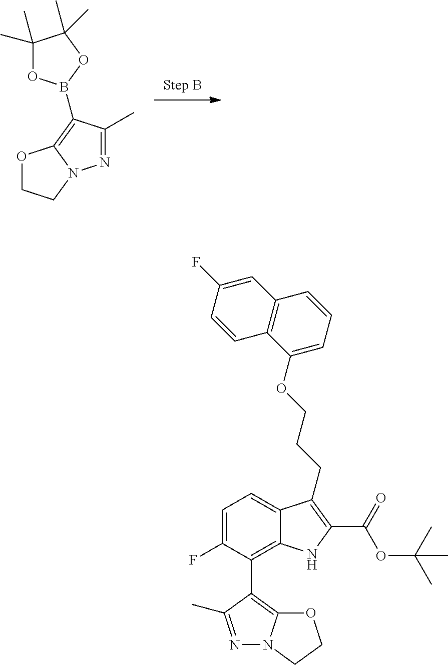

To a stirred solution of tert-butyl 7-bromo-6-fluoro-3-(3-((6-fluoronaphthalen-1-yl)oxy)propyl)-1H-indole-2-carboxylate (700 mg, 1.359 mmol) in dioxane (12 mL) and water (3 mL) were added 6-methyl-7-(4,4,5,5-tetramethyl-1,3,2-dioxaborolan-2-yl)-2,3-dihydropyrazolo[5,1-b]oxazole (679 mg, 2.718 mmol) and potassium phosphate (862 mg, 4.07 mmol). The mixture was deoxygenated with argon and to it was added [1,1′-Bis(di-tert-butylphosphino)ferrocene]dichloropalladium(II) (Pd118) (176 mg, 0.0.272 mmol) under argon atmosphere. The reaction mixture was then heated to reflux for 16 h. After complete consumption of the starting material (monitored by TLC and LCMS) the reaction mixture was filtered through Celite® pad and the solvents were evaporated under reduced pressure get the crude material. It was then diluted with EtOAc and washed successively with water and brine. The organic layer was dried over Na₂SO₄ and evaporated under reduced pressure to get the crude compound, which was then purified using flash chromatography (SiO₂, 30% EtOAc in DCM) to get tert-butyl 6-fluoro-3-(3-((6-fluoronaphthalen-1-yl)oxy)propyl)-7-(6-methyl-2,3-dihydropyrazolo[5,1-b]oxazol-7-yl)-1H-indole-2-carboxylate (650 mg, 1.162 mmol, 85.5%) as brown solid.

LCMS (ESI+): m/z 560.5[M+H]⁺

### Step C

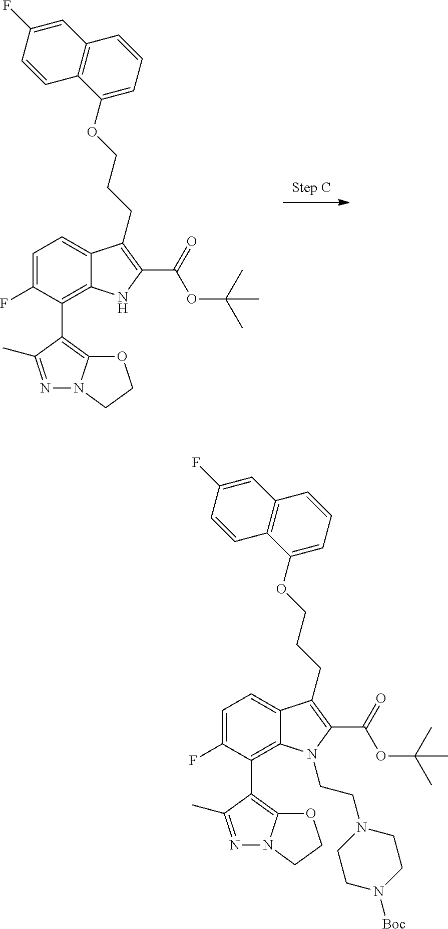 [SMILES: Cc1nn2c(c1-c1c(F)ccc3c(CCCOc4cccc5cc(F)ccc45)c(C(=O)OC(C)(C)C)[nH]c13)OCC2 ; Cc1nn2c(c1-c1c(F)ccc3c(CCCOc4cccc5cc(F)ccc45)c(C(=O)OC(C)(C)C)n(CCN4CCN(C(=O)OC(C)(C)C)CC4)c13)OCC2]

To a well stirred solution of tert-butyl 6-fluoro-3-(3-((6-fluoronaphthalen-1-yl)oxy)propyl)-7-(6-methyl-2,3-dihydropyrazolo[5,1-b]oxazol-7-yl)-1H-indole-2-carboxylate (650 mg, 1.162 mmol) in DMF (8 mL) was added tert-butyl 4-(2-chloroethyl)piperazine-1-carboxylate (377 mg, 1.512 mmol), followed by Cs₂CO₃ (566 mg, 1.74 mmol) in DMF and the mixture was allowed to stir at 90° C. for 16 h under nitrogen. After complete consumption of the starting material the reaction mixture was diluted with EtOAc and washed successively with water and brine. The organic layer was dried over Na₂SO₄ and evaporated under reduced pressure to get the crude compound, which was then purified with column chromatography (SiO₂, 40% EtOAc in DCM) to get tert-butyl 1-(2-(4-(tert-butoxycarbonyl)piperazin-1-yl)ethyl)-6-fluoro-3-(3-((6-fluoronaphthalen-1-yl)oxy)propyl)-7-(6-methyl-2,3-dihydropyrazolo[5,1-b]oxazol-7-yl)-1H-indole-2-carboxylate (630 mg, 0.82 mmol, 70.19%) as white solid.

LCMS (ESI+): m/z 772.5 [M+H]⁺

### Step D

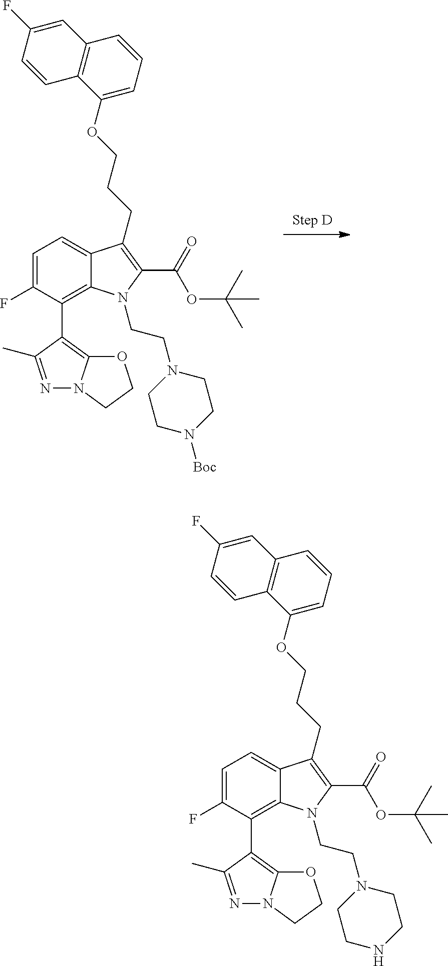 [SMILES: Cc1nn2c(c1-c1c(F)ccc3c(CCCOc4cccc5cc(F)ccc45)c(C(=O)OC(C)(C)C)n(CCN4CCN(C(=O)OC(C)(C)C)CC4)c13)OCC2 ; Cc1nn2c(c1-c1c(F)ccc3c(CCCOc4cccc5cc(F)ccc45)c(C(=O)OC(C)(C)C)n(CCN4CCNCC4)c13)OCC2]

To a stirred solution tert-butyl 1-(2-(4-(tert-butoxycarbonyl)piperazin-1-yl)ethyl)-6-fluoro-3-(3-((6-fluoronaphthalen-1-yl)oxy)propyl)-7-(6-methyl-2,3-dihydropyrazolo[5,1-b]oxazol-7-yl)-1H-indole-2-carboxylate (600 mg, 0.78 mmol) in dioxane (20 mL) was added 4M HCl in dioxane (10 mL) dropwise at 0° C. and the mixture was allowed to stir for 2 h under nitrogen at same temperature. Where upon LCMS indicated the reaction was complete, the reaction mixture was quenched by dropwise addition of cold 1M NaOH solution at 0° C. to tune the pH to 7-8. The aqueous layer was extracted 2-3 times with dichloromethane. The combined organics were dried over Na₂SO₄ and concentrated in vacuum to get the crude compound which was then purified with preparative HPLC (H₂O:MeCN+0.1% FA) to afford tert-butyl 6-fluoro-3-(3-((6-fluoronaphthalen-1-yl)oxy)propyl)-7-(6-methyl-2,3-dihydropyrazolo[5,1-b]oxazol-7-yl)-1-(2-(piperazin-1-yl)ethyl)-1H-indole-2-carboxylate (115 mg, 0.17 mmol, 22%) as white solid.

LCMS (ESI+): m/z 672.3 [M+H]⁺

### Step E

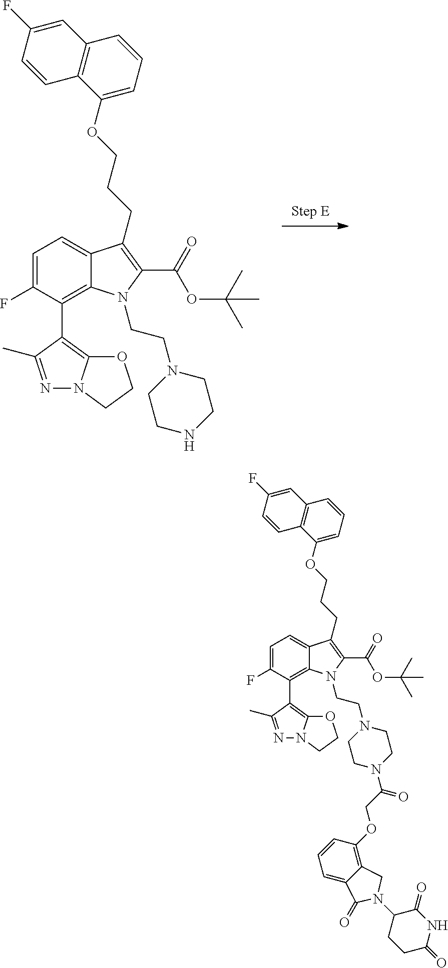 [SMILES: Cc1nn2c(c1-c1c(F)ccc3c(CCCOc4cccc5cc(F)ccc45)c(C(=O)OC(C)(C)C)n(CCN4CCNCC4)c13)OCC2 ; Cc1nn2c(c1-c1c(F)ccc3c(CCCOc4cccc5cc(F)ccc45)c(C(=O)OC(C)(C)C)n(CCN4CCN(C(=O)COc5cccc6c5CN(C5CCC(=O)NC5=O)C6=O)CC4)c13)OCC2]

To a well stirred solution of 2-{[2-(2,6-dioxopiperidin-3-yl)-1-oxo-2,3-dihydro-1H-isoindol-4-yl]oxy}acetic acid (26.1 mg, 0.082 mmol) and HATU (31.1 mg, 0.082 mmol) in DMF (0.744 mL) in 0° C. was added DIPEA (0.039 mL, 0.223 mmol) and after 15 minutes tert-butyl 6-fluoro-3-{3-[(6-fluoronaphthalen-1-yl)oxy]propyl}-7-{6-methyl-2H,3H-pyrazolo[3,2-b][1,3]oxazol-7-yl}-1-[2-(piperazin-1-yl)ethyl]-1H-indole-2-carboxylate (50.0 mg, 0.074 mmol). The mixture was allowed to stir under nitrogen for 15 minutes. After complete consumption of the starting material the reaction mixture diluted with DCM, washed successively with brine. Organic layer was dried over Na₂SO₄ and evaporated under reduced pressure. Desired product was purified using preparative TLC (SiO₂, 5% of MeOH in DCM). Tert-butyl 1-{2-[4-(2-{[2-(2,6-dioxopiperidin-3-yl)-1-oxo-2,3-dihydro-1H-isoindol-4-yl]oxy}acetyl)piperazin-1-yl]ethyl}-6-fluoro-3-{3-[(6-fluoronaphthalen-1-yl)oxy]propyl}-7-{6-methyl-2H,3H-pyrazolo[3,2-b][1,3]oxazol-7-yl}-1H-indole-2-carboxylate (13.7 mg, 0.014 mmol, 18.9%) was used directly to the next step.

LCMS (ESI+): m/z 972.4 [M+H]⁺

### Step F

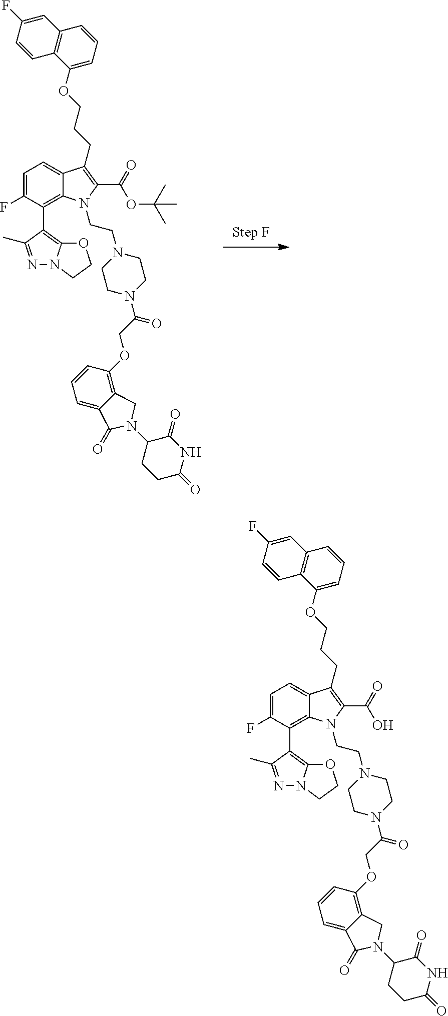 [SMILES: Cc1nn2c(c1-c1c(F)ccc3c(CCCOc4cccc5cc(F)ccc45)c(C(=O)OC(C)(C)C)n(CCN4CCN(C(=O)COc5cccc6c5CN(C5CCC(=O)NC5=O)C6=O)CC4)c13)OCC2 ; Cc1nn2c(c1-c1c(F)ccc3c(CCCOc4cccc5cc(F)ccc45)c(C(=O)O)n(CCN4CCN(C(=O)COc5cccc6c5CN(C5CCC(=O)NC5=O)C6=O)CC4)c13)OCC2]

To solution of tert-butyl 1-{2-[4-(2-{[2-(2,6-dioxopiperidin-3-yl)-1-oxo-2,3-dihydro-1H-isoindol-4-yl]oxy}acetyl)piperazin-1-yl]ethyl}-6-fluoro-3-{3-[(6-fluoronaphthalen-1-yl)oxy]propyl}-7-{6-methyl-2H,3H-pyrazolo[3,2-b][1,3]oxazol-7-yl}-1H-indole-2-carboxylate (13.7 mg, 0.014 mmol) in DCM (0.108 mL) was added TFA (0.108 mL, 1.409 mmol), and mixture was stirred for overnight at room temperature. Upon completion, the mixture was diluted with DCM and water was added, and organic solvent was removed under vacuum. Crude was purified by preparative HPLC (H₂O:MeCN with 0.1% FA) to give a corresponding 1-{2-[4-(2-{[2-(2,6-dioxopiperidin-3-yl)-1-oxo-2,3-dihydro-1H-isoindol-4-yl]oxy}acetyl)piperazin-1-yl]ethyl}-6-fluoro-3-{3-[(6-fluoronaphthalen-1-yl)oxy]propyl}-7-{6-methyl-2H,3H-pyrazolo[3,2-b][1,3]oxazol-7-yl}-1H-indole-2-carboxylic acid (5.7 mg, 0.006 mmol, 41.9%) as a white solid.

LCMS (ESI+): m/z 916.35 [M+H]⁺

¹H NMR (500 MHz, DMSO) δ=10.65 (s, 1H), 8.21 (dd, J=9.2, 5.8, 1H), 7.70 (dd, J=8.7, 5.4, 1H), 7.58 (dd, J=10.4, 2.6, 1H), 7.47-7.37 (m, 3H), 7.36-7.28 (m, 2H), 7.14 (dd, J=8.2, 0.8, 1H), 6.95 (dd, J=9.7, 8.7, 1H), 6.86 (dd, J=5.6, 3.0, 1H), 5.14-5.01 (m, 3H), 4.88 (s, 2H), 4.45 (td, J=7.0, 3.1, 2H), 4.40 (d, J=17.1, 1H), 4.36-4.29 (m, 2H), 4.29-4.20 (m, 3H), 3.38-3.32 (m, 4H), 3.32-3.24 (m, 2H), 2.88 (ddd, J=17.4, 13.5, 5.5, 1H), 2.64-2.57 (m, 1H), 2.48-2.38 (m, 1H), 2.28-2.10 (m, 8H), 2.09-2.00 (m, 1H), 1.95 (s, 3H).

### Example 144. 6-Chloro-1-(2-(4-(2-((2-(2,6-dioxopiperidin-3-yl)-1-oxoisoindolin-4-yl)oxy)acetyl)piperazin-1-yl)ethyl)-3-(3-((1,2,3,4-tetrahydro-1,4-methanonaphthalen-5-yl)oxy)propyl)-7-(1,3,5-trimethyl-1H-pyrazol-4-yl)-1H-indole-2-carboxylic acid (352)

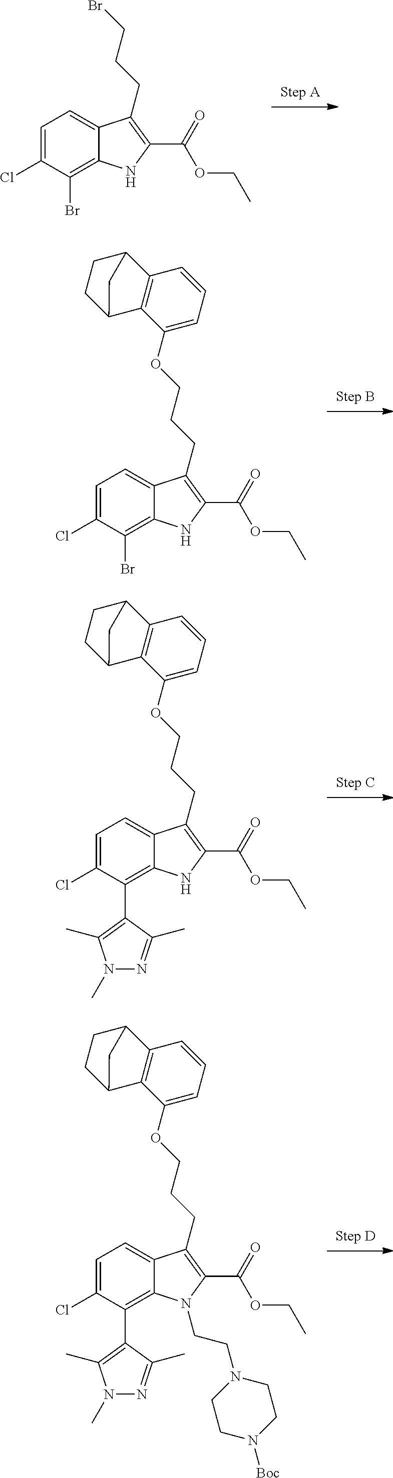

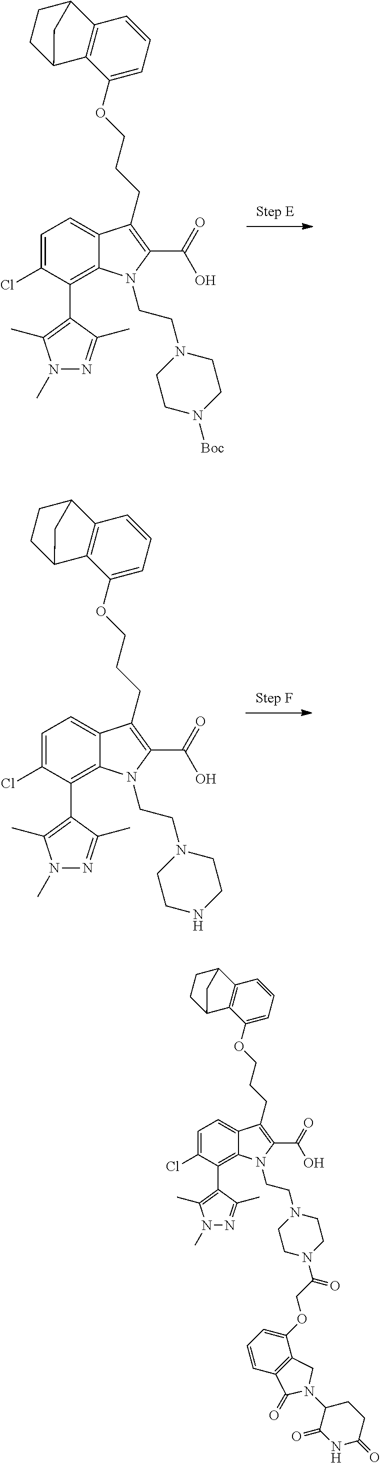 [SMILES: Cc1nn(C)c(C)c1-c1c(Cl)ccc2c(CCCOc3cccc4c3C3CCC4C3)c(C(=O)O)n(CCN3CCN(C(=O)OC(C)(C)C)CC3)c12 ; Cc1nn(C)c(C)c1-c1c(Cl)ccc2c(CCCOc3cccc4c3C3CCC4C3)c(C(=O)O)n(CCN3CCNCC3)c12 ; Cc1nn(C)c(C)c1-c1c(Cl)ccc2c(CCCOc3cccc4c3C3CCC4C3)c(C(=O)O)n(CCN3CCN(C(=O)COc4cccc5c4CN(C4CCC(=O)NC4=O)C5=O)CC3)c12]

### Step A

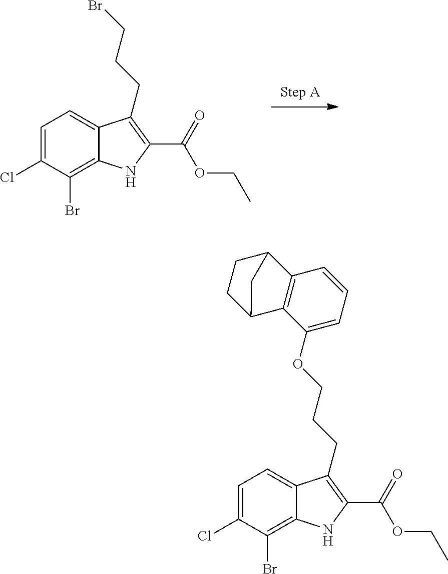 [SMILES: CCOC(=O)c1[nH]c2c(Br)c(Cl)ccc2c1CCCBr ; CCOC(=O)c1[nH]c2c(Br)c(Cl)ccc2c1CCCOc1cccc2c1C1CCC2C1]

To a solution of ethyl 7-bromo-3-(3-bromopropyl)-6-chloro-1H-indole-2-carboxylate (400 mg, 0.94 mmol) in DMF (3 mL) was added 1,2,3,4-tetrahydro-1,4-methanonaphthalen-5-ol (190 mg, 1.19 mmol), KI (197 mg, 1.19 mmol) and Cs₂CO₃ (1.15 g, 3.53 mmol) at room temperature under nitrogen. The reaction mixture was allowed to stir at 70° C. for 16 h. After complete consumption of the starting material (monitored by TLC and LCMS), the reaction mixture was diluted with EtOAc, washed successively with water and brine, the organic layer was dried over Na₂SO₄ and evaporated under reduced pressure to get the crude compound which was purified by column chromatography (SiO₂, 5% EtOAc in hexane) to get ethyl 7-bromo-6-chloro-3-(3-((1,2,3,4-tetrahydro-1,4-methanonaphthalen-5-yl)oxy)propyl)-1H-indole-2-carboxylate (300 mg, 0.597 mmol, 63%) as off brown solid.

LCMS (ESI+): m/z 500.0 & 502.0 [M+H]⁺

### Step B

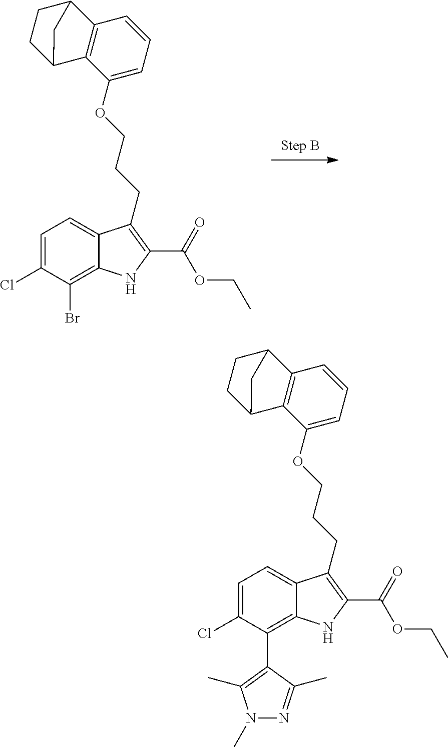

To a solution of ethyl 7-bromo-6-chloro-3-(3-((1,2,3,4-tetrahydro-1,4-methanonaphthalen-5-yl)oxy)propyl)-1H-indole-2-carboxylate (300 mg, 0.597 mmol) in dioxane (4 mL) and water (1 mL) were added 1,3,5-trimethyl-4-(4,4,5,5-tetramethyl-1,3,2-dioxaborolan-2-yl)-1H-pyrazole (424 mg, 1.796 mmol) and K₂CO₃ (331 mg, 2.395 mmol). The mixture was deoxygenated with argon and to it was added Pd(dppf)Cl₂ (73 mg, 0.09 mmol) under argon atmosphere. Then the reaction mixture was heated under reflux for 16 h. After complete consumption of the starting material (monitored by TLC and LCMS), the reaction mixture was filtered through a Celite® pad and the solvents were evaporated under reduced pressure. It was then diluted with EtOAc and washed successively with water and brine. The organic layer was dried over Na₂SO₄ and evaporated under reduced pressure to get the crude compound, which was then purified by column chromatography (SiO₂, 30% EtOAc in DCM) to get ethyl 6-chloro-3-(3-((1,2,3,4-tetrahydro-1,4-methanonaphthalen-5-yl)oxy)propyl)-7-(1,3,5-trimethyl-1H-pyrazol-4-yl)-1H-indole-2-carboxylate (220 mg, 0.413 mmol, 69%) as brown solid.

LCMS (ESI+): m/z 532.4 [M+H]⁺

### Step C

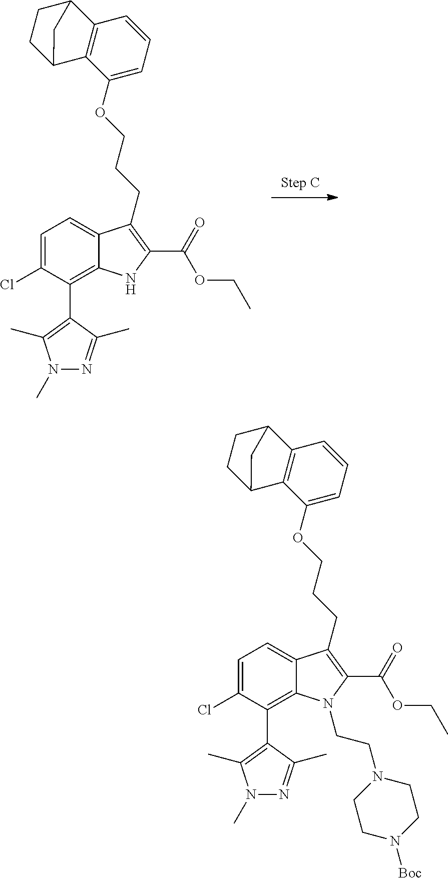 [SMILES: CCOC(=O)c1[nH]c2c(-c3c(C)nn(C)c3C)c(Cl)ccc2c1CCCOc1cccc2c1C1CCC2C1 ; CCOC(=O)c1c(CCCOc2cccc3c2C2CCC3C2)c2ccc(Cl)c(-c3c(C)nn(C)c3C)c2n1CCN1CCN(C(=O)OC(C)(C)C)CC1]

To a well stirred solution of ethyl 6-chloro-3-(3-((1,2,3,4-tetrahydro-1,4-methanonaphthalen-5-yl)oxy)propyl)-7-(1,3,5-trimethyl-1H-pyrazol-4-yl)-1H-indole-2-carboxylate (220 mg, 0.413 mmol) in DMF (3 mL) was added tert-butyl 4-(2-chloroethyl)piperazine-1-carboxylate (134 mg, 0.539 mmol) followed by Cs₂CO₃ (201 mg, 0.617 mmol) in DMF and the mixture was allowed to stir at 90° C. for 16 h under nitrogen. After complete consumption of the starting material (monitored by TLC and LCMS), the reaction mixture was diluted with EtOAc and washed successively with water and brine. The organic layer was dried over Na₂SO₄ and evaporated under reduced pressure to get the crude compound, which was then purified by column chromatography (SiO₂, 50% EtOAc in DCM) to get ethyl 1-(2-(4-(tert-butoxycarbonyl)piperazin-1-yl)ethyl)-6-chloro-3-(3-((1,2,3,4-tetrahydro-1,4-methanonaphthalen-5-yl)oxy)propyl)-7-(1,3,5-trimethyl-1H-pyrazol-4-yl)-1H-indole-2-carboxylate (200 mg, 0.269 mmol, 65%) as brown solid.

LCMS (ESI+): m/z 744.3 [M+H]⁺.

### Step D

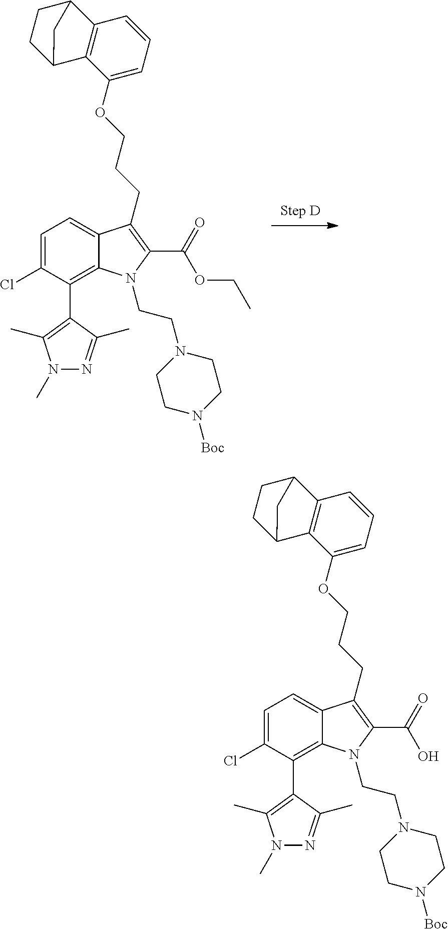 [SMILES: CCOC(=O)c1c(CCCOc2cccc3c2C2CCC3C2)c2ccc(Cl)c(-c3c(C)nn(C)c3C)c2n1CCN1CCN(C(=O)OC(C)(C)C)CC1 ; Cc1nn(C)c(C)c1-c1c(Cl)ccc2c(CCCOc3cccc4c3C3CCC4C3)c(C(=O)O)n(CCN3CCN(C(=O)OC(C)(C)C)CC3)c12]

Ethyl 1-(2-(4-(tert-butoxycarbonyl)piperazin-1-yl)ethyl)-6-chloro-3-(3-((1,2,3,4-tetrahydro-1,4-methanonaphthalen-5-yl)oxy)propyl)-7-(1,3,5-trimethyl-1H-pyrazol-4-yl)-1H-indole-2-carboxylate (200 mg, 0.269 mmol) was dissolved in EtOH (10 mL) and solution of NaOH (43 mg, 1.077 mmol) in water (2 mL) was added to it. The mixture was heated under reflux for 6 h. After complete consumption of the starting material (monitored by TLC and LCMS), the reaction mixture was cooled down to room temperature and solvents were evaporated under reduced pressure. It was then diluted with water and washed with EtOAc. Aqueous layer was carefully acidified using 1M HCl to pH=3, extracted with DCM, dried over Na₂SO₄, filtered, and concentrated in vacuo to afford 170 mg crude of 1-(2-(4-(tert-butoxycarbonyl)piperazin-1-yl)ethyl)-6-chloro-3-(3-((1,2,3,4-tetrahydro-1,4-methanonaphthalen-5-yl)oxy)propyl)-7-(1,3,5-trimethyl-1H-pyrazol-4-yl)-1H-indole-2-carboxylic acid as brown solid.

LCMS (ESI+): m/z 716.35 [M+H]⁺

### Step E

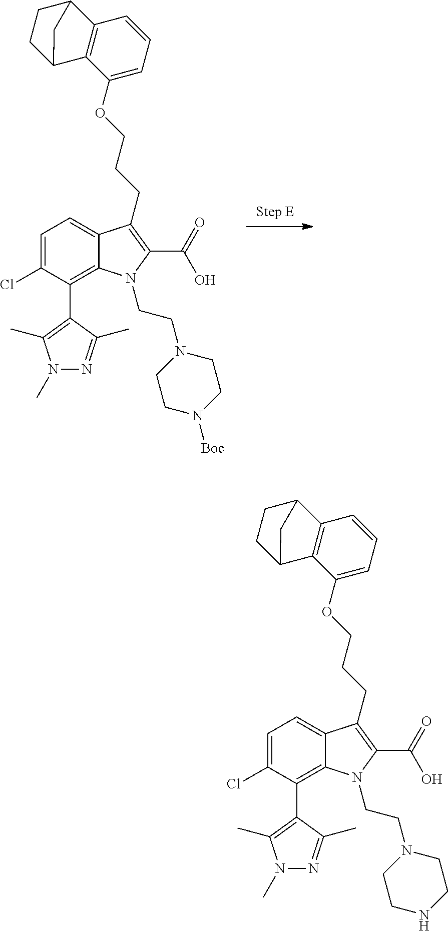 [SMILES: Cc1nn(C)c(C)c1-c1c(Cl)ccc2c(CCCOc3cccc4c3C3CCC4C3)c(C(=O)O)n(CCN3CCN(C(=O)OC(C)(C)C)CC3)c12 ; Cc1nn(C)c(C)c1-c1c(Cl)ccc2c(CCCOc3cccc4c3C3CCC4C3)c(C(=O)O)n(CCN3CCNCC3)c12]

To a solution of 1-(2-(4-(tert-butoxycarbonyl)piperazin-1-yl)ethyl)-6-chloro-3-(3-((1,2,3,4-tetrahydro-1,4-methanonaphthalen-5-yl)oxy)propyl)-7-(1,3,5-trimethyl-1H-pyrazol-4-yl)-1H-indole-2-carboxylic acid (170 mg, crude) in DCM (7 mL) was added TFA in DCM (25%, 3 mL) and the mixture was allowed to stir at room temperature for 16 h under nitrogen. After complete consumption of the starting material (monitored by TLC and LCMS), the reaction mixture was evaporated under reduced pressure to get the crude compound which was purified by reverse phase chromatography (C18, 20 mM ammonium acetate in H₂O:MeCN) to get 6-chloro-1-(2-(piperazin-1-yl)ethyl)-3-(3-((1,2,3,4-tetrahydro-1,4-methanonaphthalen-5-yl)oxy)propyl)-7-(1,3,5-trimethyl-1H-pyrazol-4-yl)-1H-indole-2-carboxylic acid (76 mg, 0.123 mmol, 46% over 2 steps) as white solid.

LCMS (ESI+): m/z 616.6 [M+H]⁺

### Step F

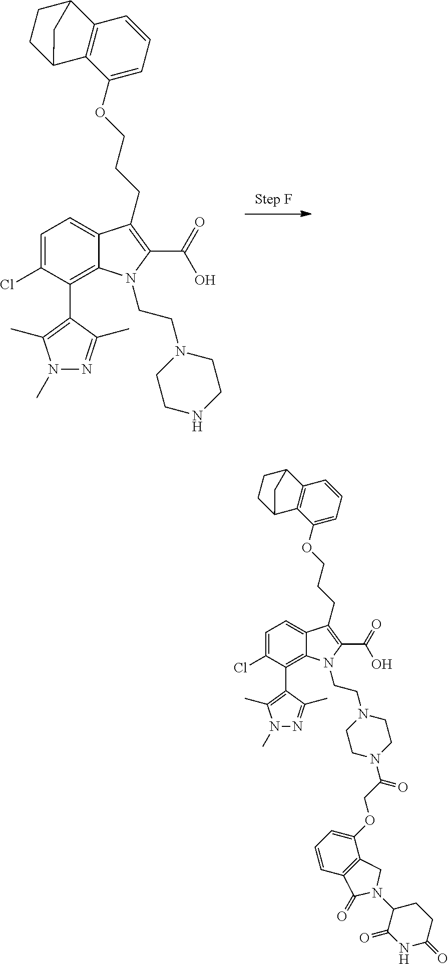 [SMILES: Cc1nn(C)c(C)c1-c1c(Cl)ccc2c(CCCOc3cccc4c3C3CCC4C3)c(C(=O)O)n(CCN3CCNCC3)c12 ; Cc1nn(C)c(C)c1-c1c(Cl)ccc2c(CCCOc3cccc4c3C3CCC4C3)c(C(=O)O)n(CCN3CCN(C(=O)COc4cccc5c4CN(C4CCC(=O)NC4=O)C5=O)CC3)c12]

2-{[2-(2,6-Dioxopiperidin-3-yl)-1-oxo-2,3-dihydro-1H-isoindol-4-yl]oxy}acetic acid (25.8 mg, 0.081 mmol) was dissolved in dry DMF (1.0 mL) and DIPEA (0.028 mL, 0.162 mmol) was added, followed by HATU (14.8 mg, 0.039 mmol). Reaction was stirred at room temperature for 1 h and 6-chloro-1-(2-(piperazin-1-yl)ethyl)-3-(3-((1,2,3,4-tetrahydro-1,4-methanonaphthalen-5-yl)oxy)propyl)-7-(1,3,5-trimethyl-1H-pyrazol-4-yl)-1H-indole-2-carboxylic acid (20.0 mg, 0.032 mmol) was added in DMSO (1.0 mL). Reaction (monitored with LCMS) was stirred for 1 h. After that time starting material was still present in the mixture. Second portion of 2-{[2-(2,6-dioxopiperidin-3-yl)-1-oxo-2,3-dihydro-1H-isoindol-4-yl]oxy}acetic acid (25.8 mg, 0.081 mmol), DIPEA (0.028 mL, 0.162 mmol) and HATU (14.8 mg, 0.039 mmol) in dry DMF (1.0 mL) was stirred at room temperature for 1 h and both solutions were combined. After next 1 h, full conversion was observed. The solution was purified with preparative HPLC (C18, H₂O:MeCN+0.1% FA) to give 6-chloro-1-(2-(4-(2-((2-(2,6-dioxopiperidin-3-yl)-1-oxoisoindolin-4-yl)oxy)acetyl)piperazin-1-yl)ethyl)-3-(3-((1,2,3,4-tetrahydro-1,4-methanonaphthalen-5-yl)oxy)propyl)-7-(1,3,5-trimethyl-1H-pyrazol-4-yl)-1H-indole-2-carboxylic acid (20.4 mg, 0.022 mmol, 68%) as white solid.

LCMS (ESI+): m/z 916.30 [M+H]⁺

¹H NMR (500 MHz, DMSO, 353 K) δ 10.64 (s, 1H), 7.66 (d, J=8.5 Hz, 1H), 7.44 (t, J=7.8 Hz, 1H), 7.33 (d, J=7.4 Hz, 1H), 7.21 (d, J=8.5 Hz, 1H), 7.15 (d, J=8.2 Hz, 1H), 6.94 (dd, J=8.3, 7.2 Hz, 1H), 6.77 (m, 1H), 6.62 (dd, J=8.3, 0.8 Hz, 1H), 5.05 (dd, J=13.1, 5.2 Hz, 1H), 4.89 (s, 2H), 4.40 (d, J=17.1 Hz, 1H), 4.32 (dd, J=17.2, 1.5 Hz, 1H), 4.30-4.23 (m, 1H), 4.23-4.15 (m, 1H), 4.04 (qt, J=9.9, 6.5 Hz, 2H), 3.77-3.74 (m, 3H), 3.56-3.52 (m, 1H), 3.39-3.34 (m, 4H), 3.33-3.31 (m, 1H), 3.18 (dd, J=8.4, 6.4 Hz, 2H), 2.88 (ddd, J=17.4, 13.4, 5.5 Hz, 1H), 2.66-2.59 (m, 1H), 2.48-2.40 (m, 1H), 2.19-2.12 (m, 5H), 2.11-2.03 (m, 4H), 2.02 (s, 3H), 1.90 (s, 3H), 1.89-1.82 (m, 2H), 1.59 (dp, J=8.3, 2.0 Hz, 1H), 1.45 (dt, J=8.6, 1.5 Hz, 1H), 1.08-1.00 (m, 2H).

### Example 145. 6-Chloro-1-(2-(4-(2-((2-(2,6-dioxopiperidin-3-yl)-1-oxoisoindolin-4-yl)oxy)acetyl)piperazin-1-yl)ethyl)-3-(3-(methyl(naphthalen-1-yl)amino)propyl)-7-(1,3,5-trimethyl-1H-pyrazol-4-yl)-1H-indole-2-carboxylic acid (353)

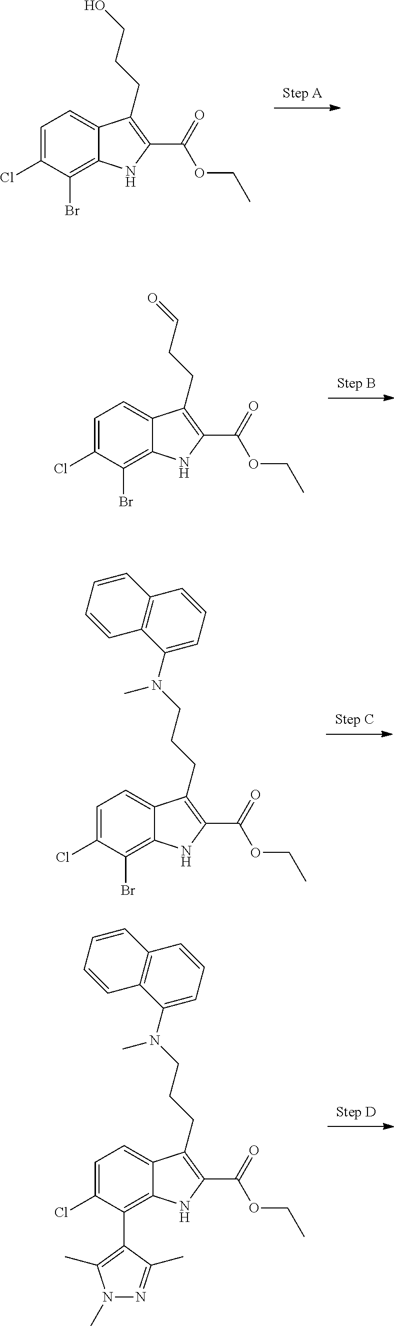

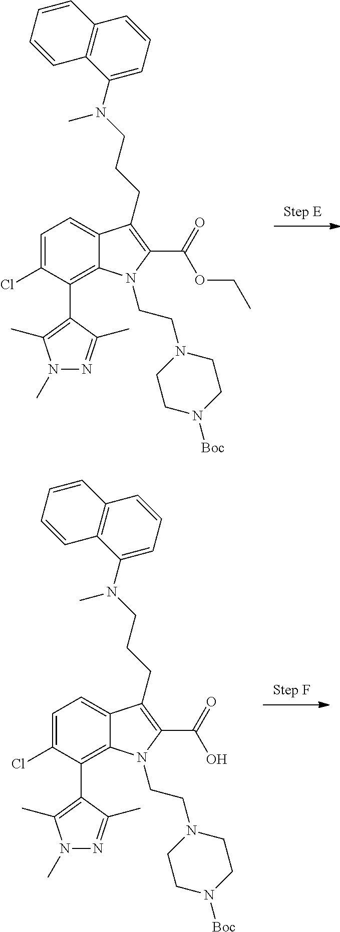

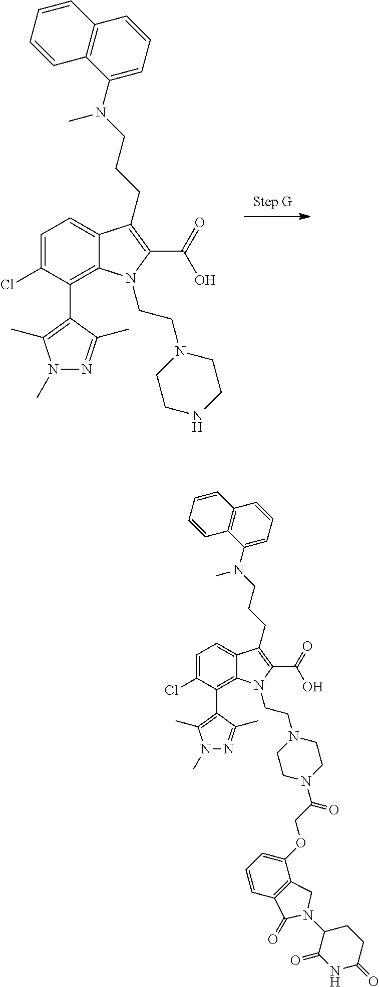 [SMILES: Cc1nn(C)c(C)c1-c1c(Cl)ccc2c(CCCN(C)c3cccc4ccccc34)c(C(=O)O)n(CCN3CCNCC3)c12 ; Cc1nn(C)c(C)c1-c1c(Cl)ccc2c(CCCN(C)c3cccc4ccccc34)c(C(=O)O)n(CCN3CCN(C(=O)COc4cccc5c4CN(C4CCC(=O)NC4=O)C5=O)CC3)c12]

### Step A

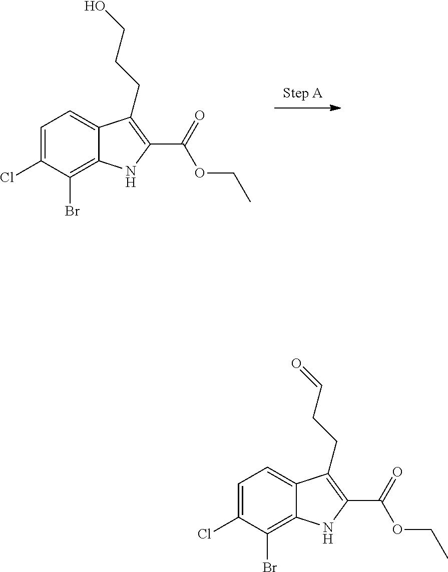 [SMILES: CCOC(=O)c1[nH]c2c(Br)c(Cl)ccc2c1CCCO ; CCOC(=O)c1[nH]c2c(Br)c(Cl)ccc2c1CCC=O]

To a stirred solution of ethyl 7-bromo-6-chloro-3-(3-hydroxypropyl)-1H-indole-2-carboxylate (1 g, 2.77 mmol) in DCM (20 mL), was added Dess-Martin Periodinane (2.3 g, 5.42 mmol) portion wise at 0° C. under nitrogen. The reaction mixture was allowed to stir at room temperature for 3 h. After complete consumption of the starting material (monitored by TLC), few drops of water were added, the solid was filtered through Celite® pad and washed with DCM thoroughly. The filtrate was evaporated under reduced pressure to get the crude compound which was purified by column chromatography (SiO₂, 20% EtOAc in DCM) to afford ethyl 7-bromo-6-chloro-3-(3-oxopropyl)-1H-indole-2-carboxylate (700 mg, 1.95 mmol, 70%) as white solid.

### Step B

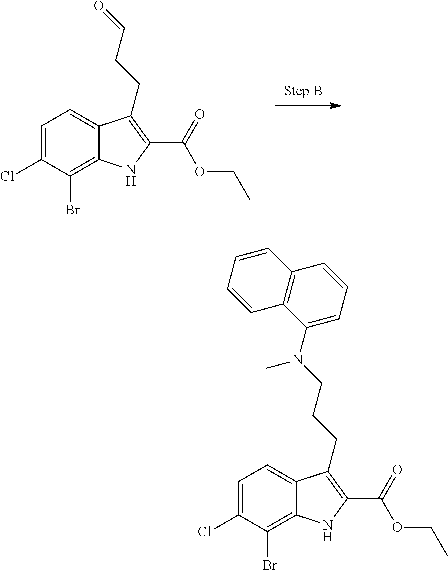 [SMILES: CCOC(=O)c1[nH]c2c(Br)c(Cl)ccc2c1CCC=O ; CCOC(=O)c1[nH]c2c(Br)c(Cl)ccc2c1CCCN(C)c1cccc2ccccc12]

To a stirred solution of ethyl 7-bromo-6-chloro-3-(3-oxopropyl)-1H-indole-2-carboxylate (700 mg, 1.95 mmol) and N-methylnaphthalen-1-amine (306 mg, 1.95 mmol) in THE (10 mL) was added dibutyl tindichloride (893 mg, 2.94 mmol) followed by DIPEA (0.7 mL, 3.933 mmol). pH of the reaction mixture was adjusted to −8˜9 and the reaction mixture was allowed to stir at ambient temperature for 30 min under nitrogen. Phenyl silane (6 mg, 0.055 mmol) was then added to the reaction mixture and it was allowed to stir at 85° C. for 16 h under nitrogen. After complete consumption of the starting material (monitored by TLC and LCMS), the volatiles were evaporated under reduced pressure. The reaction mixture was diluted with EtOAc and washed successively with water and brine. The organic layer was evaporated under reduced pressure to afford crude compound which was purified by column chromatography (SiO₂, 5% EtOAc in hexane) to afford ethyl 7-bromo-6-chloro-3-(3-(methyl(naphthalen-1-yl)amino)propyl)-1H-indole-2-carboxylate (300 mg, 0.60 mmol, 31%) as brown solid.

LCMS (ESI+): m/z 500.8 [M+H]⁺

### Step C

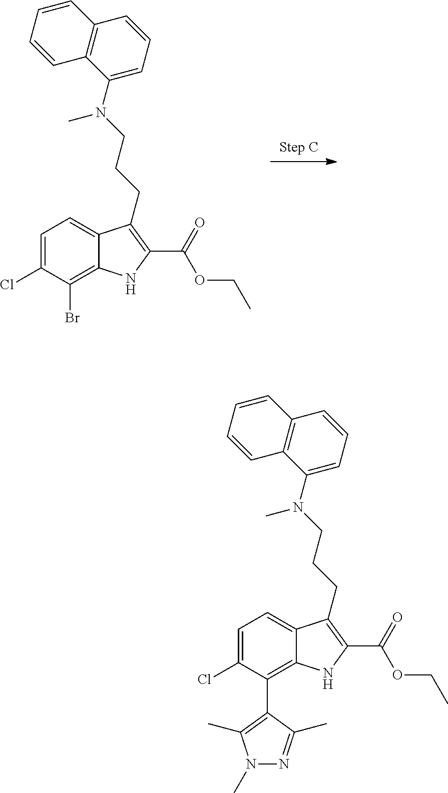 [SMILES: CCOC(=O)c1[nH]c2c(Br)c(Cl)ccc2c1CCCN(C)c1cccc2ccccc12 ; CCOC(=O)c1[nH]c2c(-c3c(C)nn(C)c3C)c(Cl)ccc2c1CCCN(C)c1cccc2ccccc12]

To a solution of ethyl 7-bromo-6-chloro-3-(3-(methyl(naphthalen-1-yl)amino)propyl)-1H-indole-2-carboxylate (300 mg, 0.60 mmol) in dioxane (10 mL) and water (2 mL) were added 1,3,5-trimethyl-4-(4,4,5,5-tetramethyl-1,3,2-dioxaborolan-2-yl)-1H-pyrazole (426 mg, 1.80 mmol) and K₃PO₄ (510 mg, 2.4 mmol). The mixture was deoxygenated with argon and to it was added PdCl₂(dtbpf) (58 mg, 0.089 mmol) under argon atmosphere. Then the reaction mixture was heated under reflux for 16 h. After complete consumption of the starting material (monitored by TLC and LCMS), the reaction mixture was filtered through Celite® pad and the solvents were evaporated under reduced pressure get the crude material. It was then diluted with EtOAc, washed successively with water and brine, the organic layer was dried over Na₂SO₄ and evaporated under reduced pressure to get the crude compound, which was then purified by column chromatography (SiO₂, 30% EtOAc in DCM) to get ethyl 6-chloro-3-(3-(methyl(naphthalen-1-yl)amino)propyl)-7-(1,3,5-trimethyl-1H-pyrazol-4-yl)-1H-indole-2-carboxylate (250 mg, 0.47 mmol, 78%) as brown solid.

LCMS (ESI+): m/z 529.2 [M+H]⁺

### Step D

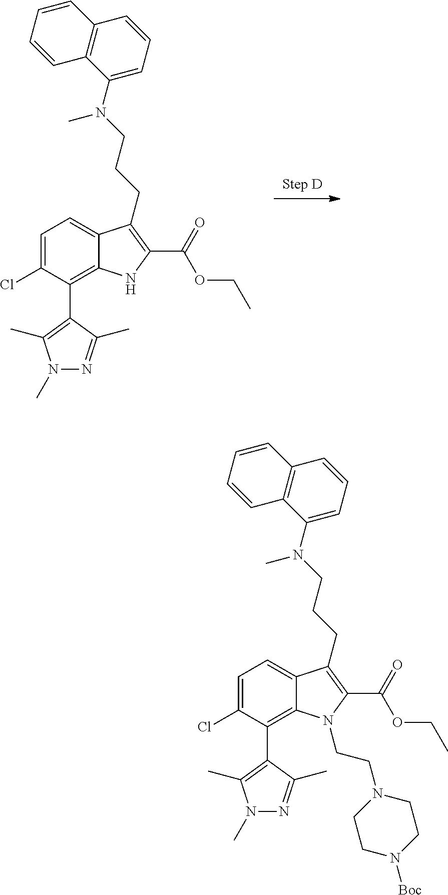 [SMILES: CCOC(=O)c1[nH]c2c(-c3c(C)nn(C)c3C)c(Cl)ccc2c1CCCN(C)c1cccc2ccccc12 ; CCOC(=O)c1c(CCCN(C)c2cccc3ccccc23)c2ccc(Cl)c(-c3c(C)nn(C)c3C)c2n1CCN1CCN(C(=O)OC(C)(C)C)CC1]

To a solution of ethyl 6-chloro-3-(3-(methyl(naphthalen-1-yl)amino)propyl)-7-(1,3,5-trimethyl-1H-pyrazol-4-yl)-1H-indole-2-carboxylate (250 mg, 0.47 mmol) in DMF (4 mL) was added tert-butyl 4-(2-chloroethyl)piperazine-1-carboxylate (176 mg, 0.71 mmol) followed by Cs₂CO₃ (230 mg, 0.71 mmol) in DMF and the mixture was allowed to stir at 90° C. for 16 h under nitrogen. After complete consumption of the starting material (monitored by TLC and LCMS), the reaction mixture was diluted with EtOAc and washed successively with water and brine. The organic layer was dried over Na₂SO₄ and evaporated under reduced pressure. Crude compound was purified by column chromatography (SiO₂, 50% EtOAc in DCM) to get ethyl 1-(2-(4-(tert-butoxycarbonyl)piperazin-1-yl)ethyl)-6-chloro-3-(3-(methyl(naphthalen-1-yl)amino)propyl)-7-(1,3,5-trimethyl-1H-pyrazol-4-yl)-1H-indole-2-carboxylate (200 mg, 0.27 mmol, 57%) as brown solid.

LCMS (ESI+): m/z 741.4 [M+H]⁺

### Step E

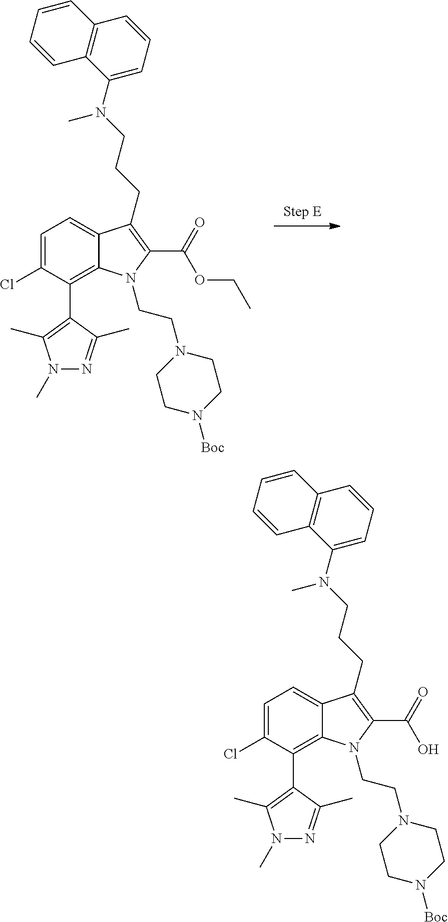 [SMILES: CCOC(=O)c1c(CCCN(C)c2cccc3ccccc23)c2ccc(Cl)c(-c3c(C)nn(C)c3C)c2n1CCN1CCN(C(=O)OC(C)(C)C)CC1 ; Cc1nn(C)c(C)c1-c1c(Cl)ccc2c(CCCN(C)c3cccc4ccccc34)c(C(=O)O)n(CCN3CCN(C(=O)OC(C)(C)C)CC3)c12]

Ethyl 1-(2-(4-(tert-butoxycarbonyl)piperazin-1-yl)ethyl)-6-chloro-3-(3-(methyl(naphthalen-1-yl)amino)propyl)-7-(1,3,5-trimethyl-1H-pyrazol-4-yl)-1H-indole-2-carboxylate (200 mg, 0.27 mmol) was dissolved in EtOH (10 mL) and solution of NaOH (43 mg, 1.08 mmol) in water (2 mL) was added to it. The mixture was heated under reflux for 6 h. After complete consumption of the starting material (monitored by TLC and LCMS), the reaction mixture was cooled down to room temperature and solvents were evaporated under reduced pressure. It was then diluted with EtOAc and washed with water. Aqueous layer was carefully acidified using 1M HCl to pH=3, extracted with DCM, dried over Na₂SO₄, filtered, and concentrated in vacuo to afford 160 mg crude of 1-(2-(4-(tert-butoxycarbonyl)piperazin-1-yl)ethyl)-6-chloro-3-(3-(methyl(naphthalen-1-yl)amino)propyl)-7-(1,3,5-trimethyl-1H-pyrazol-4-yl)-1H-indole-2-carboxylic acid as brown solid.

LCMS (ESI+): m/z 713.5 [M+H]⁺

### Step F

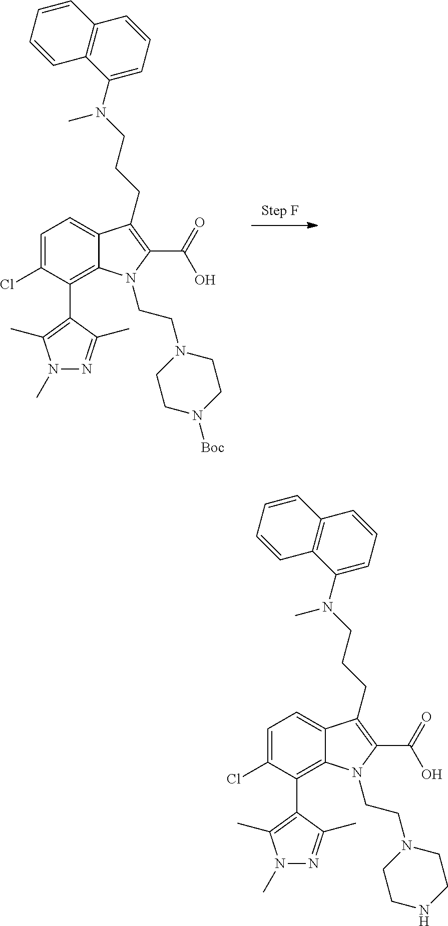 [SMILES: Cc1nn(C)c(C)c1-c1c(Cl)ccc2c(CCCN(C)c3cccc4ccccc34)c(C(=O)O)n(CCN3CCN(C(=O)OC(C)(C)C)CC3)c12 ; Cc1nn(C)c(C)c1-c1c(Cl)ccc2c(CCCN(C)c3cccc4ccccc34)c(C(=O)O)n(CCN3CCNCC3)c12]

To a solution of 1-(2-(4-(tert-butoxycarbonyl)piperazin-1-yl)ethyl)-6-chloro-3-(3-(methyl(naphthalen-1-yl)amino)propyl)-7-(1,3,5-trimethyl-1H-pyrazol-4-yl)-1H-indole-2-carboxylic acid (160 mg, crude) in DCM (7 mL) was added 4M HCl in dioxane (3 mL) and the mixture was allowed to stir at room temperature for 16 h under nitrogen. After complete consumption of the starting material (monitored by TLC and LCMS), the reaction mixture was evaporated under reduced pressure to get the crude compound which was purified by reverse phase chromatography (C18, 10 mM ammonium acetate in H₂O:MeCN) to get 6-chloro-3-(3-(methyl(naphthalen-1-yl)amino)propyl)-1-(2-(piperazin-1-yl)ethyl)-7-(1,3,5-trimethyl-1H-pyrazol-4-yl)-1H-indole-2-carboxylic acid (60 mg, 0.098 mmol, 36% over 2 steps) of as white solid.

LCMS (ESI+): m/z 613.45 [M+H]⁺

### Step G

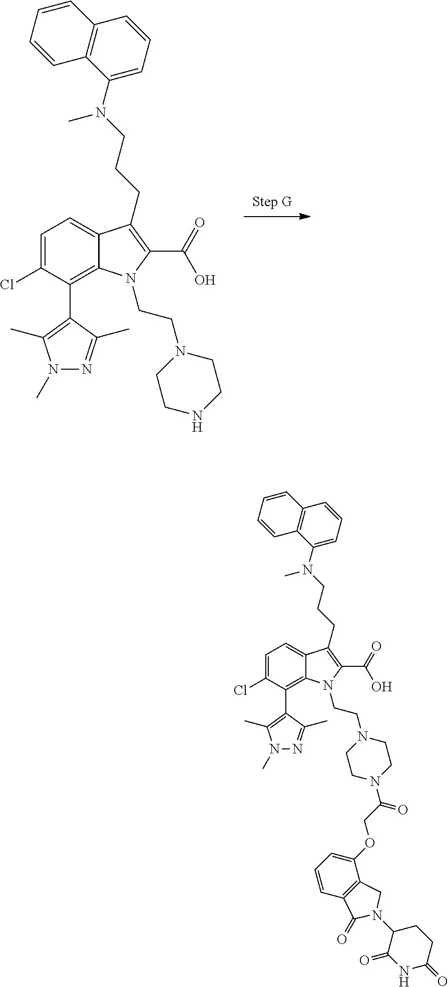 [SMILES: Cc1nn(C)c(C)c1-c1c(Cl)ccc2c(CCCN(C)c3cccc4ccccc34)c(C(=O)O)n(CCN3CCNCC3)c12 ; Cc1nn(C)c(C)c1-c1c(Cl)ccc2c(CCCN(C)c3cccc4ccccc34)c(C(=O)O)n(CCN3CCN(C(=O)COc4cccc5c4CN(C4CCC(=O)NC4=O)C5=O)CC3)c12]

2-{[2-(2,6-Dioxopiperidin-3-yl)-1-oxo-2,3-dihydro-1H-isoindol-4-yl]oxy}acetic acid (31.1 mg, 0.098 mmol) was dissolved in DMF (6.1 mL) under argon atmosphere and DIPEA (0.026 mL, 0.147 mmol) along with HATU (35.3 mg, 0.093 mmol) was added. Reaction was stirred at room temperature for 1 h under argon. To it, 6-chloro-3-(3-(methyl(naphthalen-1-yl)amino)propyl)-1-(2-(piperazin-1-yl)ethyl)-7-(1,3,5-trimethyl-1H-pyrazol-4-yl)-1H-indole-2-carboxylic acid (30.0 mg, 0.049 mmol) was added dropwise as a solution in DMSO (1.6 mL) and DIPEA (0.026 mL, 0.147 mmol). Reaction (monitored with LCMS) was stirred for 20 min at room temperature under argon. After complete consumption of the starting material, the mixture was concentrated under reduced pressure, diluted with DMSO and purified with preparative HPLC (C18, H₂O:MeCN+0.1% FA) to get 6-chloro-1-(2-(4-(2-((2-(2,6-dioxopiperidin-3-yl)-1-oxoisoindolin-4-yl)oxy)acetyl)piperazin-1-yl)ethyl)-3-(3-(methyl(naphthalen-1-yl)amino)propyl)-7-(1,3,5-trimethyl-1H-pyrazol-4-yl)-1H-indole-2-carboxylic acid (30.2 mg, 0.033 mmol, 67%) as white solid.

LCMS (ESI+): m/z 913.3 [M+H]⁺

¹H NMR (500 MHz, DMSO, 353 K) δ 10.64 (s, 1H), 8.15-8.09 (m, 1H), 7.86-7.79 (m, 1H), 7.56-7.49 (m, 2H), 7.47-7.40 (m, 2H), 7.39-7.31 (m, 3H), 7.16 (t, J=8.4 Hz, 2H), 7.11 (dd, J=7.5, 1.1 Hz, 1H), 5.05 (dd, J=13.0, 5.2 Hz, 1H), 4.87 (s, 2H), 4.40 (d, J=17.1 Hz, 1H), 4.36-4.29 (m, 1H), 4.27-4.18 (m, 1H), 4.18-4.10 (m, 1H), 3.75 (d, J=1.9 Hz, 3H), 3.33-3.27 (m, 4H), 3.16-3.12 (m, 2H), 3.08-3.06 (m, 2H), 2.88 (ddd, J=17.4, 13.4, 5.5 Hz, 1H), 2.83 (s, 3H), 2.66-2.59 (m, 1H), 2.48-2.40 (m, 1H), 2.09-2.01 (m, 7H), 2.00 (s, 3H), 2.00-1.95 (m, 2H), 1.88 (s, 3H).

### Example 146: 6-Chloro-1-(2-(4-(2-((2-(2,6-dioxopiperidin-3-yl)-1-oxoisoindolin-4-yl)oxy)acetyl)piperazin-1-yl)ethyl)-3-(3-((7-fluoronaphthalen-1-yl)oxy)propyl)-7-(1,3,5-trimethyl-1H-pyrazol-4-yl)-1H-indole-2-carboxylic acid (354)

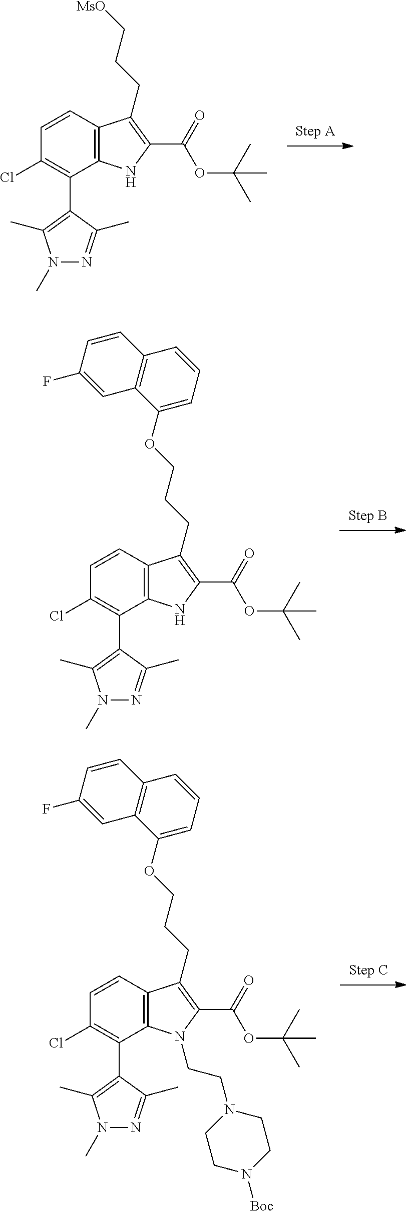

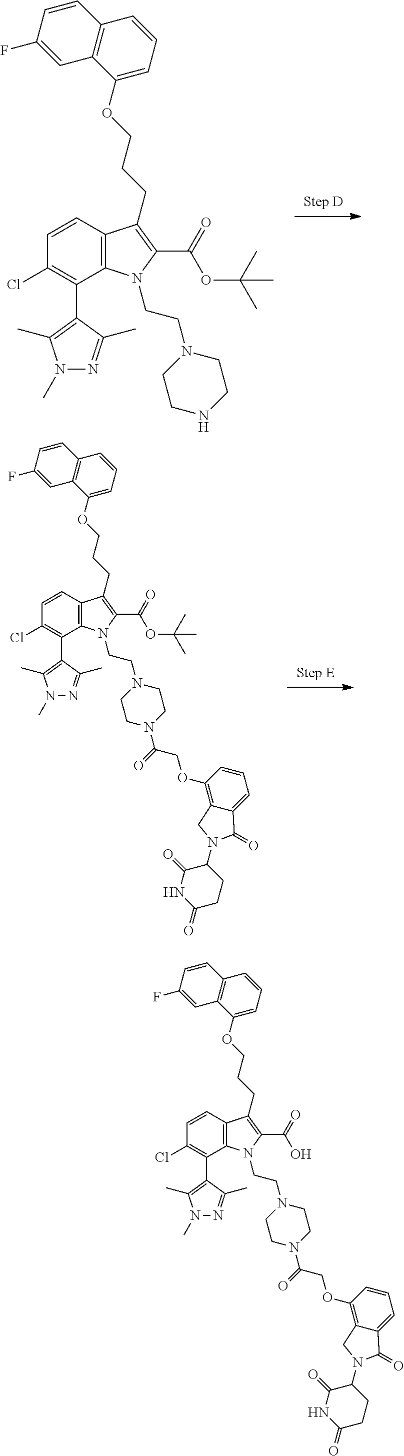 [SMILES: Cc1nn(C)c(C)c1-c1c(Cl)ccc2c(CCCOc3cccc4ccc(F)cc34)c(C(=O)OC(C)(C)C)n(CCN3CCNCC3)c12 ; Cc1nn(C)c(C)c1-c1c(Cl)ccc2c(CCCOc3cccc4ccc(F)cc34)c(C(=O)OC(C)(C)C)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4ccc(F)cc34)c(C(=O)O)n(CCN3CCN(C(=O)COc4cccc5c4CN(C4CCC(=O)NC4=O)C5=O)CC3)c12]

### Step A

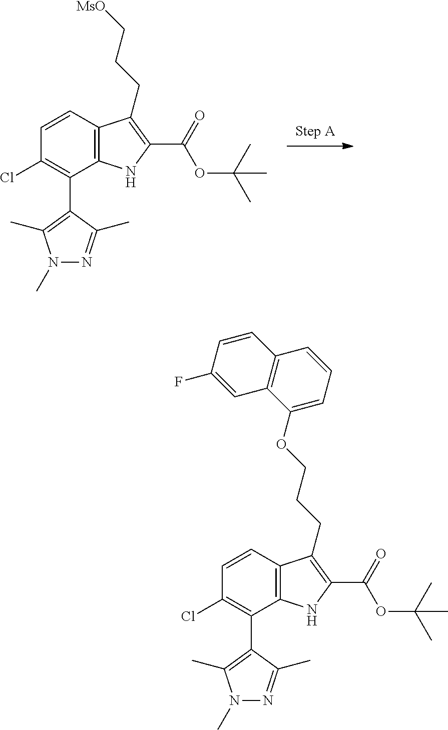 [SMILES: Cc1nn(C)c(C)c1-c1c(Cl)ccc2c(CCCOS(C)(=O)=O)c(C(=O)OC(C)(C)C)[nH]c12 ; Cc1nn(C)c(C)c1-c1c(Cl)ccc2c(CCCOc3cccc4ccc(F)cc34)c(C(=O)OC(C)(C)C)[nH]c12]

To a well stirred solution of tert-butyl 6-chloro-3-(3-((methylsulfonyl)oxy)propyl)-7-(1,3,5-trimethyl-1H-pyrazol-4-yl)-1H-indole-2-carboxylate (537 mg, 1.086 mmol) in DMF (6 mL) was added 7-fluoronaphthalen-1-ol (220 mg, 1.358 mmol) followed by Cs₂CO₃ (1.3 g, 4.074 mmol) and KI (225 mg, 1.358 mmol). The mixture was allowed to stir at 90° C. for 16 h under nitrogen. After complete consumption of the starting material the reaction mixture was diluted with EtOAc and washed successively with water and brine. The organic layer was dried over Na₂SO₄ and evaporated under reduced pressure to get the crude compound, which was then purified with column chromatography (SiO₂, 30% EtOAc in hexane) to get tert-butyl 6-chloro-3-(3-((7-fluoronaphthalen-1-yl)oxy)propyl)-7-(1,3,5-trimethyl-1H-pyrazol-4-yl)-1H-indole-2-carboxylate (400 mg, 0.711 mmol, 65.46%) of as off brown solid.

LCMS (ESI+): m/z 562.1 [M+H]⁺

### Step B

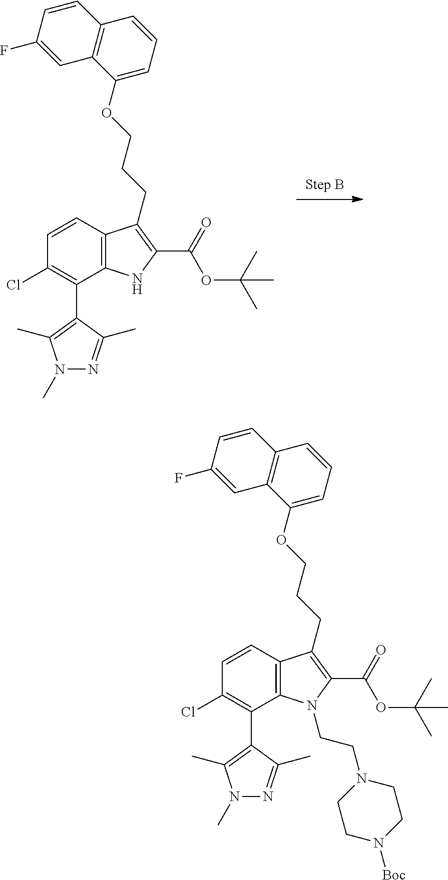 [SMILES: Cc1nn(C)c(C)c1-c1c(Cl)ccc2c(CCCOc3cccc4ccc(F)cc34)c(C(=O)OC(C)(C)C)[nH]c12 ; Cc1nn(C)c(C)c1-c1c(Cl)ccc2c(CCCOc3cccc4ccc(F)cc34)c(C(=O)OC(C)(C)C)n(CCN3CCN(C(=O)OC(C)(C)C)CC3)c12]

To a well stirred solution of tert-butyl 6-chloro-3-(3-((7-fluoronaphthalen-1-yl)oxy)propyl)-7-(1,3,5-trimethyl-1H-pyrazol-4-yl)-1H-indole-2-carboxylate (400 mg, 0.713 mmol) in DMF (5 mL) was added tert-butyl 4-(2-chloroethyl)piperazine-1-carboxylate (266 mg, 1.07 mmol) followed by Cs₂CO₃ (347 mg, 1.07 mmol) and the mixture was allowed to stir at 90° C. for 16 h under nitrogen. After complete consumption of the starting material the reaction mixture was diluted with EtOAc and washed successively with water and brine. The organic layer was dried over Na₂SO₄ and evaporated under reduced pressure to get the crude compound, which was then purified by column chromatography (SiO₂, 50% EtOAc in DCM) to get of tert-butyl 1-(2-(4-(tert-butoxycarbonyl)piperazin-1-yl)ethyl)-6-chloro-3-(3-((7-fluoronaphthalen-1-yl)oxy)propyl)-7-(1,3,5-trimethyl-1H-pyrazol-4-yl)-1H-indole-2-carboxylate (450 mg, 0.582 mmol, 81.5%) as white solid.

LCMS (ESI+): m/z 774.1 [M+H]⁺

### Step C

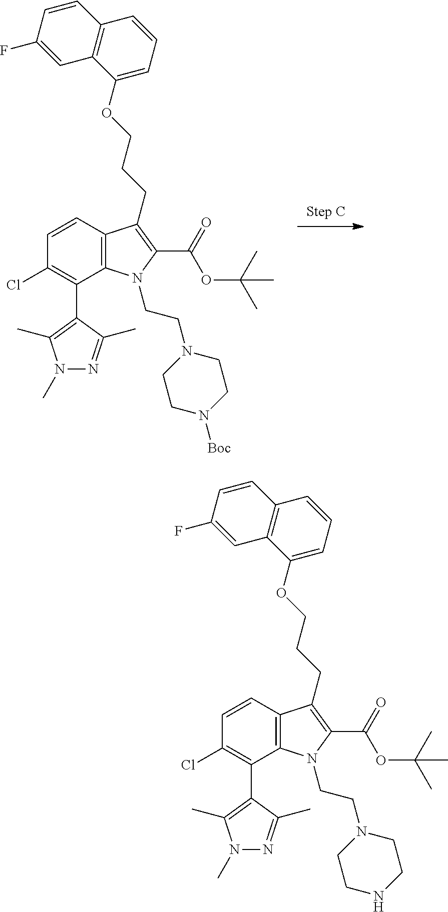

tert-Butyl 1-(2-(4-(tert-butoxycarbonyl)piperazin-1-yl)ethyl)-6-chloro-3-(3-((7-fluoronaphthalen-1-yl)oxy)propyl)-7-(1,3,5-trimethyl-1H-pyrazol-4-yl)-1H-indole-2-carboxylate (400 mg, 0.517 mmol) dissolved in DCM (8 mL) at 0° C. was added 4M HCl in dioxane (3 mL) and the mixture stirred for 2 h under nitrogen at same temperature. After complete consumption of the starting material the reaction mixture was poured in to cold 1M NaOH solution and extracted several times with dichloromethane. The combined organics were dried over Na₂SO₄ and concentrated in vacuo to get the crude compound which was then purified with column chromatography (amine SiO₂, DCM:MeOH, 10%) to get of tert-butyl 6-chloro-3-(3-((7-fluoronaphthalen-1-yl)oxy)propyl)-1-(2-(piperazin-1-yl)ethyl)-7-(1,3,5-trimethyl-1H-pyrazol-4-yl)-1H-indole-2-carboxylate (200 mg, 0.297 mmol, 57.4%) as white solid.

LCMS (ESI+): m/z 674.5 [M+H]⁺

### Step D

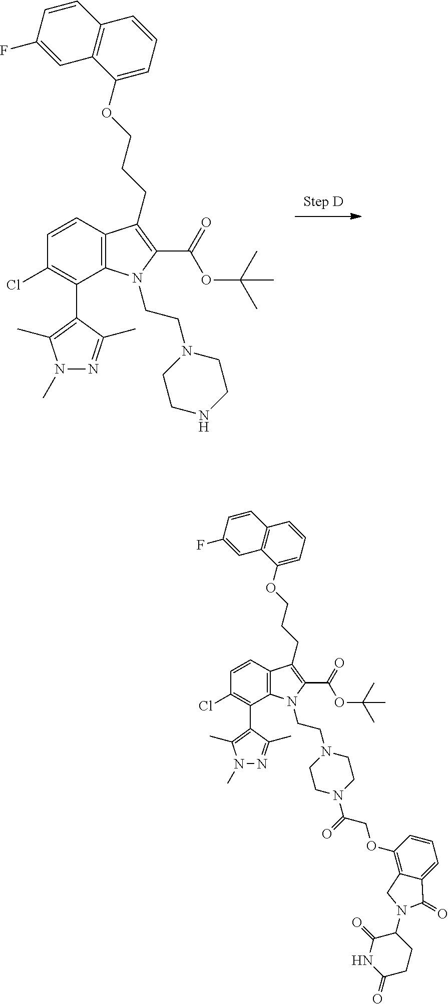 [SMILES: Cc1nn(C)c(C)c1-c1c(Cl)ccc2c(CCCOc3cccc4ccc(F)cc34)c(C(=O)OC(C)(C)C)n(CCN3CCNCC3)c12 ; Cc1nn(C)c(C)c1-c1c(Cl)ccc2c(CCCOc3cccc4ccc(F)cc34)c(C(=O)OC(C)(C)C)n(CCN3CCN(C(=O)COc4cccc5c4CN(C4CCC(=O)NC4=O)C5=O)CC3)c12]

To a stirred suspension of 2-((2-(2,6-dioxopiperidin-3-yl)-1-oxoisoindolin-4-yl)oxy)acetic acid (116 mg, 0.366 mmol) in DMF (3 mL) was added HATU (231 mg, 0.609 mmol) followed by DIPEA (0.15 mL, 0.914 mmol) at 0° C. under nitrogen. The reaction mixture was then allowed to stir at ambient temperature for 15 minutes, after 15 min to it was added tert-butyl 6-chloro-3-(3-((7-fluoronaphthalen-1-yl)oxy)propyl)-1-(2-(piperazin-1-yl)ethyl)-7-(1,3,5-trimethyl-1H-pyrazol-4-yl)-1H-indole-2-carboxylate (200 mg, 0.297 mmol) in DMF (1 mL) and the mixture was allowed to stir for 1 h under nitrogen. After complete consumption of the starting material the reaction mixture was diluted with EtOAc, washed successively with water and brine, the organic layer was dried over Na₂SO₄ and evaporated under reduced pressure to get the crude compound, which was then purified by column chromatography (SiO₂, 70% EtOAc in DCM) to get tert-butyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((7-fluoronaphthalen-1-yl)oxy)propyl)-7-(1,3,5-trimethyl-1H-pyrazol-4-yl)-1H-indole-2-carboxylate (150 mg, 0.154 mmol, 51.8%) as white solid.

LCMS (ESI+): m/z 974.5 [M+H]⁺

### Step E

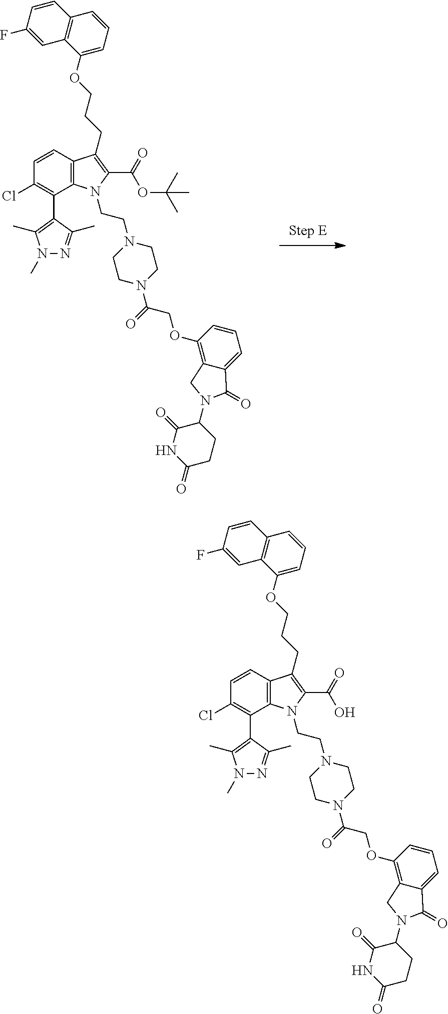 [SMILES: Cc1nn(C)c(C)c1-c1c(Cl)ccc2c(CCCOc3cccc4ccc(F)cc34)c(C(=O)OC(C)(C)C)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4ccc(F)cc34)c(C(=O)O)n(CCN3CCN(C(=O)COc4cccc5c4CN(C4CCC(=O)NC4=O)C5=O)CC3)c12]

To a stirred suspension of tert-butyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((7-fluoronaphthalen-1-yl)oxy)propyl)-7-(1,3,5-trimethyl-1H-pyrazol-4-yl)-1H-indole-2-carboxylate (150 mg, 0.154 mmol) in DCM (2 mL) was added TFA (2 mL) dropwise at 0° C. under nitrogen. The mixture was allowed to stir at room temperature for 16 h. After complete consumption of starting material the volatiles were evaporated under reduced pressure to get the crude compound, which was then purified with reverse phase preparative HPLC (20 mM ammonium bicarbonate in H₂O:MeCN) to get 6-chloro-1-(2-(4-(2-((2-(2,6-dioxopiperidin-3-yl)-1-oxoisoindolin-4-yl)oxy)acetyl)piperazin-1-yl)ethyl)-3-(3-((7-fluoronaphthalen-1-yl)oxy)propyl)-7-(1,3,5-trimethyl-1H-pyrazol-4-yl)-1H-indole-2-carboxylic acid (60 mg, 0.065 mmol, 42.2%) as off white solid.

LCMS (ESI+): m/z 918.6 [M+H]⁺

¹H NMR (400 MHz, DMSO) δ=10.98 (s, 1H), 7.96 (dd, J=9.1, 5.7, 1H), 7.76 (dd, J=10.9, 2.7, 1H), 7.73-7.65 (m, 1H), 7.49 (d, J=8.3, 1H), 7.47-7.39 (m, 2H), 7.37 (t, J=7.9, 1H), 7.31 (d, J=7.4, 1H), 7.19 (d, J=8.6, 1H), 7.11 (d, J=8.2, 1H), 6.95 (d, J=7.6, 1H), 5.11 (dd, J=13.3, 5.1, 1H), 4.95 (s, 2H), 4.38 (d, J=17.4, 1H), 4.33-4.10 (m, 5H), 3.74 (d, J=1.9, 3H), 3.29-3.19 (m, 6H), 2.98-2.84 (m, 1H), 2.63-2.52 (m, 1H), 2.46-2.38 (m, 1H), 2.26-2.16 (m, 2H), 2.14-1.94 (m, 10H), 1.86 (s, 3H).

### Synthesis of Ligase Ligand Moieties—Compound 111

### Example 147: 4-amino-2-(2,6-dioxopiperidin-3-yl)-6-(2-hydroxyethyl)isoindoline-1,3-dione (Compound 111)

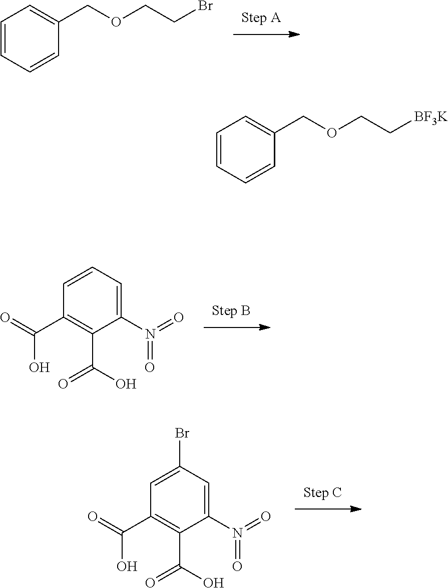

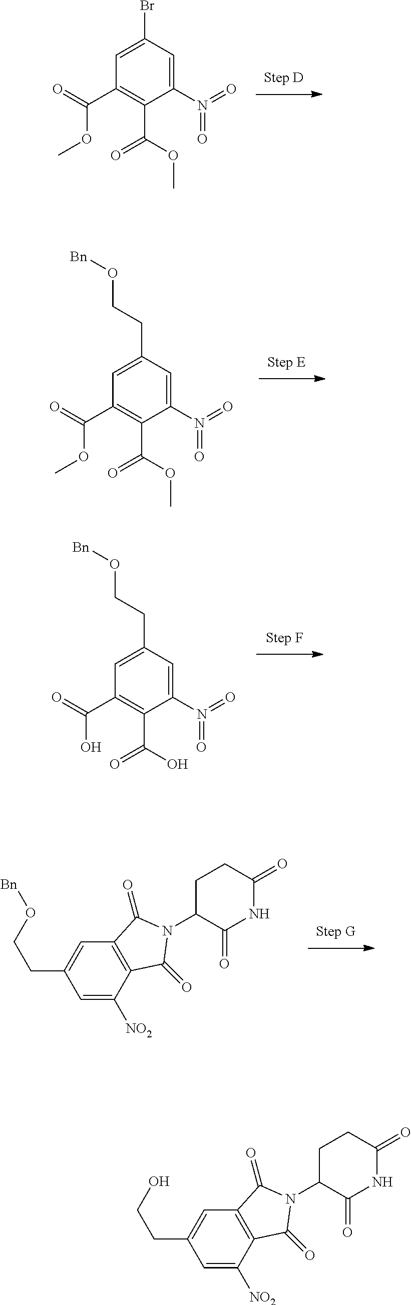 [SMILES: COC(=O)c1cc(Br)cc([N+](=O)[O-])c1C(=O)OC ; COC(=O)c1cc(CCOCc2ccccc2)cc([N+](=O)[O-])c1C(=O)OC ; O=C(O)c1cc(CCOCc2ccccc2)cc([N+](=O)[O-])c1C(=O)O ; O=C1CCC(N2C(=O)c3cc(CCOCc4ccccc4)cc([N+](=O)[O-])c3C2=O)C(=O)N1 ; O=C1CCC(N2C(=O)c3cc(CCO)cc([N+](=O)[O-])c3C2=O)C(=O)N1]

### Step A

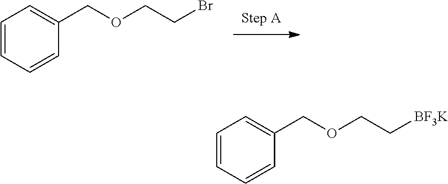

BISPIN (8.855 g, 34.869 mmol), LiOMe (1.766 g, 46.492 mmol), triphenylphosphine polymer-bound (2.3 g, 3.022 mmol) and CuI (0.433 g, 2.325 mmol) were weighed in a round-bottomed flask equipped with a stir bar, under argon atmosphere. The flask was closed with a septum, evacuated, and backfilled with nitrogen. A solution of ((2-bromoethoxy)methyl)benzene (5.0 g, 23.246 mmol) in DMF (116 ml) was added with a syringe and the resulting mixture was vigorously stirred at room temperature for 20 h. The reaction mixture was then diluted with dichloromethane and filtered through a Celite pad. The resulting solution was concentrated, poured into sat. aq NH₄Cl, and extracted with Et₂O (2×150 ml). The organic layers were washed successively with H₂O (3×100 ml) and brine (100 ml). The organic layer was dried over anhydrous Na₂SO₄, and concentrated to get the crude product which was dissolved in THE (100 ml). A saturated solution of KHF₂ (15 ml, 91.547 mmol) was added to it and the reaction mixture was stirred for 2 h in RT. The reaction mixture was dried, and resulting salt was extracted with hot acetone (2×100 ml). The organic part was concentrated and precipitation was achieved by dropwise addition of diethyl ether at 0° C. The resulting product was collected by filtration and dried to get potassium (2-benzyloxyethyl)trifluoroborate (5 g, 13.6 mmol, 59%) as white solid.

### Step B

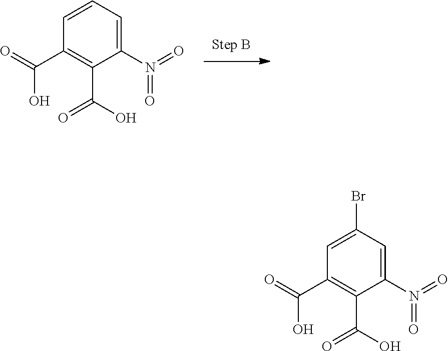 [SMILES: O=C(O)c1cccc([N+](=O)[O-])c1C(=O)O ; O=C(O)c1cc(Br)cc([N+](=O)[O-])c1C(=O)O]

To a well stirred solution of 3-Nitrophthalic acid (15 g, 71 mmol) in concentrated H2SO4 (60 ml) was added 1,3-Dibromo-5,5-dimethylhydantoin (11 g, 38.38 mmol) at 0° C. and the reaction mixture was allowed to stir at ambient temperature for 16 h. Reaction mixture was then poured into ice cold water to get the a white precipitate. The precipitate was filtered, the residue was washed with cold water several times then dried under vacuum to get 5-bromo-3-nitrophthalic acid (15 g, 51.7 mmol; 72%) as white solid.

### Step C

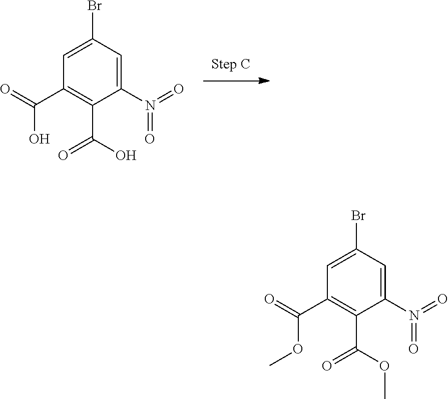 [SMILES: O=C(O)c1cc(Br)cc([N+](=O)[O-])c1C(=O)O ; COC(=O)c1cc(Br)cc([N+](=O)[O-])c1C(=O)OC]

To a solution of 5-bromo-3-nitrophthalic acid (10 g, 34.5 mmol) in DMF (5 ml) was added sodium bicarbonate (23.2 g, 276 mmol) followed by methyl iodide (12.9 ml, 206.9 mmol) and the reaction mixture was allowed to stir at 100° C. for 16 h under nitrogen. After complete consumption of the starting material the reaction mixture was diluted with ethyl acetate, washed successively with water and brine, the organic layer was dried over sodium sulphate and evaporated under reduced pressure to get the crude compound, which was then purified by column chromatography (SiO₂, 30% ethyl acetate in hexane) to get dimethyl 5-bromo-3-nitrophthalate (6 g, 21 mmol, 60%) as off white solid.

### Step D

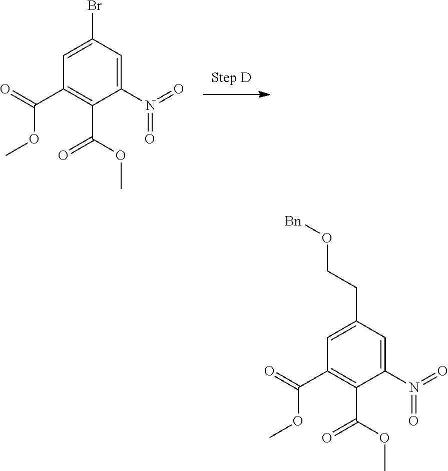 [SMILES: COC(=O)c1cc(Br)cc([N+](=O)[O-])c1C(=O)OC ; COC(=O)c1cc(CCOCc2ccccc2)cc([N+](=O)[O-])c1C(=O)OC]

A suspension of dimethyl 5-bromo-3-nitrophthalate (2.0 g, 6.289 mmol), potassium (2-benzyloxyethyl)trifluoroborate (3.805 g, 15.723 mmol) and Cs₂CO₃ (6.148 g, 18.868 mmol) in a toluene:water mixture (4:1, 30 ml) was deoxygenated using argon for 10 min. To the reaction mixture was added Pd(amphos)₂Cl₂ (0.891 g, 1.258 mmol) and it was allowed to stir at 100° C. for 16 h. After complete consumption of the starting material the volatiles were evaporated under reduced pressure to get the crude compound, which was diluted with ethyl acetate, washed successively with water and brine, the organic layer was dried over sodium sulphate and evaporated under reduced pressure to get the crude compound, which was then purified by column chromatography (SiO₂, 20% ethyl acetate in hexane) to get dimethyl 5-(2-(benzyloxy)ethyl)-3-nitrophthalate (1.6 g, 4.28 mmol, 68%) as gummy solid.

### Step E

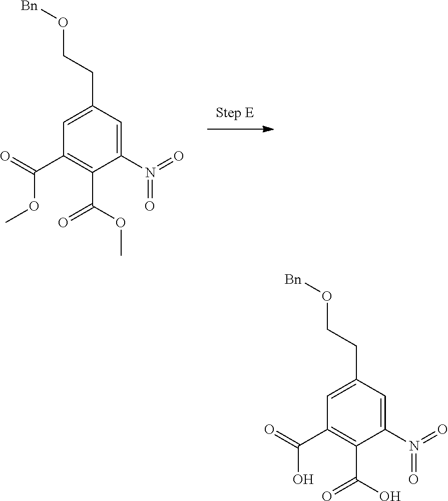 [SMILES: COC(=O)c1cc(CCOCc2ccccc2)cc([N+](=O)[O-])c1C(=O)OC ; O=C(O)c1cc(CCOCc2ccccc2)cc([N+](=O)[O-])c1C(=O)O]

To a solution of dimethyl 5-(2-(benzyloxy)ethyl)-3-nitrophthalate (1.6 g, 4.285 mmol) in methanol (30 ml) was added NaOH (1.714 g, 42.853 mmol) and the reaction mixture was then refluxed for 4 h. The reaction mixture was cooled to room temperature; volatiles were evaporated under reduced pressure. The residue was dissolved in water (100 ml) and acidified by 2M HCl solution. The aqueous part was extracted by ethyl acetate. Next, the combined organic layer was washed by water and brine, dried over anhydrous Na₂SO₄ and concentrated under reduced pressure to get 5-(2-(benzyloxy)ethyl)-3-nitrophthalic acid (1.4 g, 4.05 mmol, 94.6%) as off white solid.

### Step F

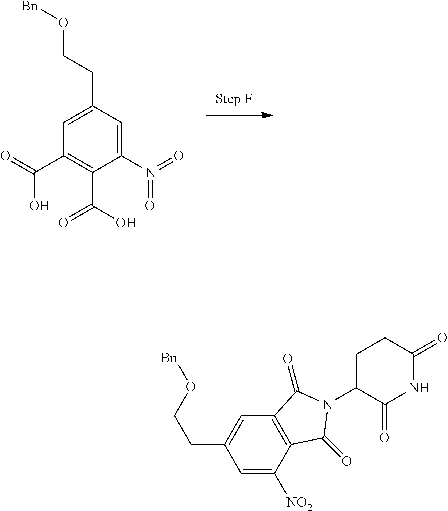 [SMILES: O=C(O)c1cc(CCOCc2ccccc2)cc([N+](=O)[O-])c1C(=O)O ; O=C1CCC(N2C(=O)c3cc(CCOCc4ccccc4)cc([N+](=O)[O-])c3C2=O)C(=O)N1]

To a solution of 5-(2-(benzyloxy)ethyl)-3-nitrophthalic acid (1.4 g, 4.06 mmol) in acetic acid (40 ml) and was added 3-aminopiperidine-2,6-dione hydrochloride (670 mg, 4.06 mmol) followed by sodium acetate (1 g, 12.17 mmol) and the reaction mixture was allowed to stir at 120° C. for 4 h under nitrogen. After complete consumption of the starting material the reaction mixture was evaporated under reduced pressure get the crude material which was then purified by column chromatography (SiO₂, 10% ethyl acetate in dichloromethane) to get 6-(2-(benzyloxy)ethyl)-2-(2,6-dioxopiperidin-3-yl)-4-nitroisoindoline-1,3-dione (1 g, 2.28 mmol, 56%) as light brown solid.

LCMS (ESI+): m/z 438.3 [M+H]⁺

### Step G

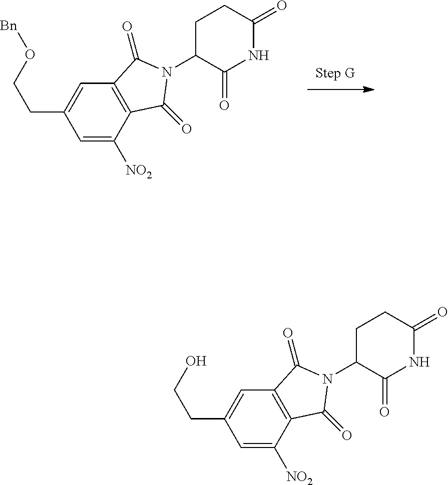 [SMILES: O=C1CCC(N2C(=O)c3cc(CCOCc4ccccc4)cc([N+](=O)[O-])c3C2=O)C(=O)N1 ; O=C1CCC(N2C(=O)c3cc(CCO)cc([N+](=O)[O-])c3C2=O)C(=O)N1]

6-(2-(benzyloxy)ethyl)-2-(2,6-dioxopiperidin-3-yl)-4-nitroisoindoline-1,3-dione (500 mg, 1.14 mmol) was dissolved in acetic acid (10 ml). To it was added 10% Pd/C (50 mg), the reaction vessel was then filled with hydrogen (using balloon) and the reaction mixture was allowed to stir at room temperature for 16 h. After complete consumption of the starting material the reaction mixture was filtered through Celite pad and evaporated under reduced pressure to get the crude compound which was purified by column chromatography (SiO₂, 5% methanol in dichloromethane) to get 4-amino-2-(2,6-dioxopiperidin-3-yl)-6-(2-hydroxyethyl)isoindoline-1,3-dione (200 mg, 0.63 mmol, 55%) as yellow solid.

LCMS (ESI+): m/z 318.2 [M+H]⁺

¹H NMR (400 MHz, DMSO) δ 11.07 (s, 1H), 6.90 (s, 1H), 6.84 (s, 1H), 6.42 (s, 2H), 5.02 (dd, J=12.9, 5.4 Hz, 1H), 4.69 (t, J=5.2 Hz, 1H), 3.61 (q, J=6.2 Hz, 2H), 2.94-2.80 (m, 1H), 2.72 (t, J=6.6 Hz, 2H), 2.62-2.52 (m, 2H), 2.06-1.94 (m, 1H).

### Synthesis of Ligase Ligand Moieties—Compounds of Formula (II) and Formula (III)

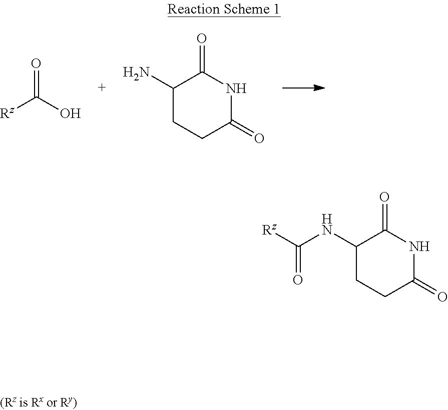

### Synthetic Conditions A

An appropriate acid (R^{z}COOH in the above Reaction Scheme 1) (1. eq), DMAP (0.04 eq), and EDC (1.2 eq) were added to a solution of 3-aminopiperidine-2,6-dione (1 eq) and N-hydroxybenzotriazole (1.2 eq) in DMF (0.5 M). The reaction mixture was stirred overnight at room temperature (20-25° C.). After removal of the solvent under reduced pressure, the crude product was purified by preparative HPLC, flash column chromatography or preparative TLC.

### Synthetic Conditions B

DIPEA (2-3 eq) was added to a solution of an appropriate acid (R^{z}COOH in the above Reaction Scheme 1), DMAP (0-0.1 eq), HATU (1.0-1.5 eq) and 3-aminopiperidine-2,6-dione hydrochloride (1.2-3.0 eq) in DMF (0.1-0.5 M). The reaction mixture was stirred overnight at room temperature (20-25° C.). After removal of the solvent under reduced pressure, the crude product was purified by preparative HPLC, flash column chromatography or preparative TLC.

### Synthetic Conditions C

CDI (1.2-2 eq) was added to a solution of an appropriate acid (R^{z}COOH in the above Reaction Scheme 1) in DMF (0.1-0.5 M) and stirred for 1 h at 50° C. After cooling to room temperature, 3-aminopiperidine-2,6-dione hydrochloride (1.2-1.5 equiv) was added and the reaction mixture was stirred overnight at room temperature (20-25° C.). After removal of the solvent under reduced pressure, the crude product was purified by preparative HPLC, flash column chromatography or preparative TLC.

TABLE 6

| Compound | | |
|---|---|---|
| number | Structure | Synthesis method |
| 1 | 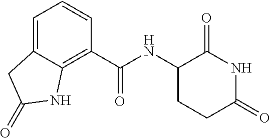 [SMILES: O=C1CCC(NC(=O)c2cccc3c2NC(=O)C3)C(=O)N1] | Synthetic Conditions A |
| 3 | 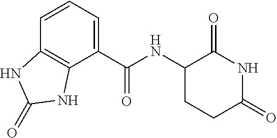 [SMILES: O=C1CCC(NC(=O)c2cccc3[nH]c(=O)[nH]c23)C(=O)N1] | Synthetic Conditions B |
| 4 | 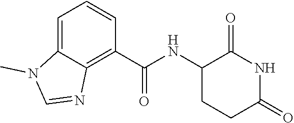 [SMILES: Cn1cnc2c(C(=O)NC3CCC(=O)NC3=O)cccc21] | Synthetic Conditions B |
| 5 | 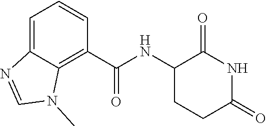 [SMILES: Cn1cnc2cccc(C(=O)NC3CCC(=O)NC3=O)c21] | Synthetic Conditions B (also described in detail below) |
| 7 | 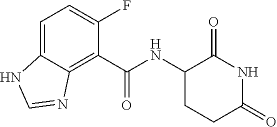 [SMILES: O=C1CCC(NC(=O)c2c(F)ccc3[nH]cnc23)C(=O)N1] | Synthetic Conditions C |
| 8 | 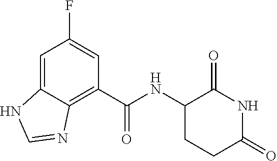 [SMILES: O=C1CCC(NC(=O)c2cc(F)cc3[nH]cnc23)C(=O)N1] | Synthetic Conditions B |
| 9 | 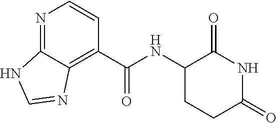 [SMILES: O=C1CCC(NC(=O)c2ccnc3[nH]cnc23)C(=O)N1] | Synthetic Conditions C |
| 10 | 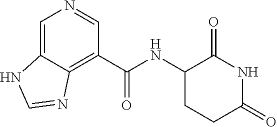 [SMILES: O=C1CCC(NC(=O)c2cncc3[nH]cnc23)C(=O)N1] | Synthetic Conditions C |
| 11 | 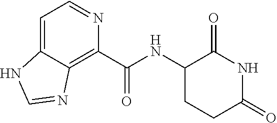 [SMILES: O=C1CCC(NC(=O)c2nccc3[nH]cnc23)C(=O)N1] | Synthetic Conditions C |
| 12 | 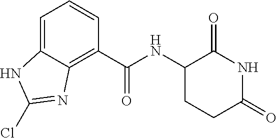 [SMILES: O=C1CCC(NC(=O)c2cccc3[nH]c(Cl)nc23)C(=O)N1] | Synthetic Conditions B |
| 14 | 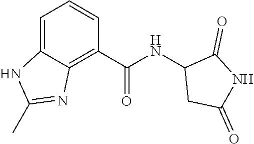 [SMILES: Cc1nc2c(C(=O)NC3CC(=O)NC3=O)cccc2[nH]1] | Synthetic Conditions C |
| 17 | 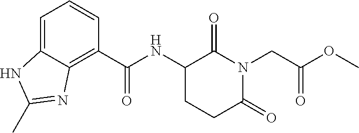 | Synthetic Conditions B |
| 22 | 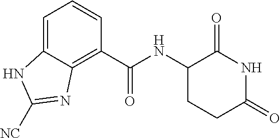 [SMILES: N#Cc1nc2c(C(=O)NC3CCC(=O)NC3=O)cccc2[nH]1] | Synthetic Conditions B |
| 23 | 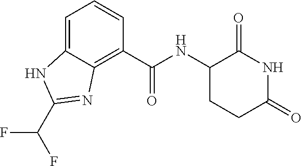 [SMILES: O=C1CCC(NC(=O)c2cccc3[nH]c(C(F)F)nc23)C(=O)N1] | Synthetic Conditions B |
| 24 | 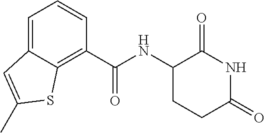 [SMILES: Cc1cc2cccc(C(=O)NC3CCC(=O)NC3=O)c2s1] | Synthetic Conditions B |
| 25 | 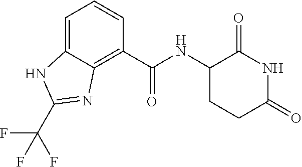 [SMILES: O=C1CCC(NC(=O)c2cccc3[nH]c(C(F)(F)F)nc23)C(=O)N1] | Synthetic Conditions B |
| 29 | 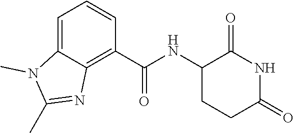 [SMILES: Cc1nc2c(C(=O)NC3CCC(=O)NC3=O)cccc2n1C] | Synthetic Conditions B |
| 30 | 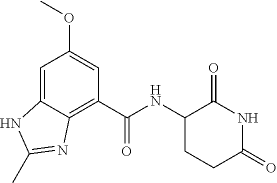 [SMILES: COc1cc(C(=O)NC2CCC(=O)NC2=O)c2nc(C)[nH]c2c1] | Synthetic Conditions B |
| 35 | 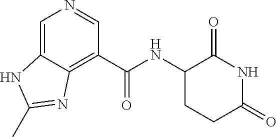 [SMILES: Cc1nc2c(C(=O)NC3CCC(=O)NC3=O)cncc2[nH]1] | Synthetic Conditions B |
| 36 | 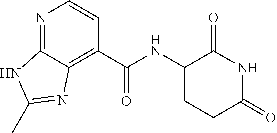 [SMILES: Cc1nc2c(C(=O)NC3CCC(=O)NC3=O)ccnc2[nH]1] | Synthetic Conditions B |
| 37 | 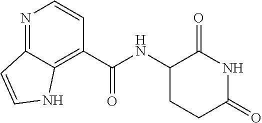 [SMILES: O=C1CCC(NC(=O)c2ccnc3cc[nH]c23)C(=O)N1] | Synthetic Conditions B |
| 38 | 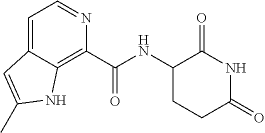 [SMILES: Cc1cc2ccnc(C(=O)NC3CCC(=O)NC3=O)c2[nH]1] | Synthetic Conditions B |
| 39 | 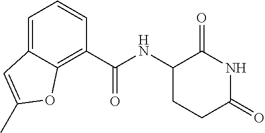 [SMILES: Cc1cc2cccc(C(=O)NC3CCC(=O)NC3=O)c2o1] | Synthetic Conditions B |
| 40 | 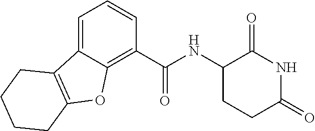 [SMILES: O=C1CCC(NC(=O)c2cccc3c4c(oc23)CCCC4)C(=O)N1] | Synthetic Conditions B |
| 41 | 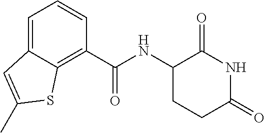 [SMILES: Cc1cc2cccc(C(=O)NC3CCC(=O)NC3=O)c2s1] | Synthetic Conditions B |
| 42 | 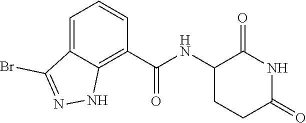 [SMILES: O=C1CCC(NC(=O)c2cccc3c(Br)n[nH]c23)C(=O)N1] | Synthetic Conditions B |
| 43 | 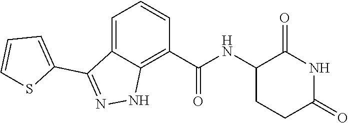 [SMILES: O=C1CCC(NC(=O)c2cccc3c(-c4cccs4)n[nH]c23)C(=O)N1] | Synthetic Conditions B |
| 45 | 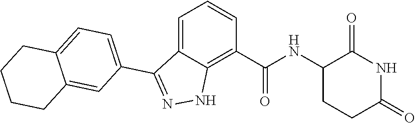 [SMILES: O=C1CCC(NC(=O)c2cccc3c(-c4ccc5c(c4)CCCC5)n[nH]c23)C(=O)N1] | Synthetic Conditions B |
| 46 | 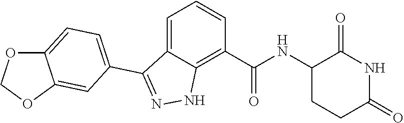 [SMILES: O=C1CCC(NC(=O)c2cccc3c(-c4ccc5c(c4)OCO5)n[nH]c23)C(=O)N1] | Synthetic Conditions B |
| 47 | 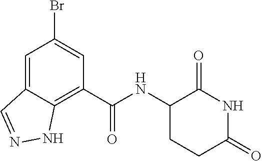 [SMILES: O=C1CCC(NC(=O)c2cc(Br)cc3cn[nH]c23)C(=O)N1] | Synthetic Conditions B |
| 48 | 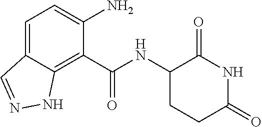 [SMILES: Nc1ccc2cn[nH]c2c1C(=O)NC1CCC(=O)NC1=O] | Synthetic Conditions B |
| 50 | 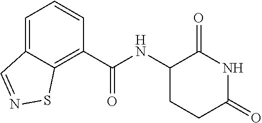 [SMILES: O=C1CCC(NC(=O)c2cccc3cnsc23)C(=O)N1] | Synthetic Conditions B |
| 51 | 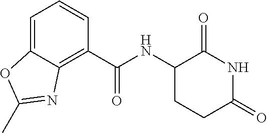 [SMILES: Cc1nc2c(C(=O)NC3CCC(=O)NC3=O)cccc2o1] | Synthetic Conditions B |
| 52 | 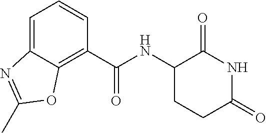 [SMILES: Cc1nc2cccc(C(=O)NC3CCC(=O)NC3=O)c2o1] | Synthetic Conditions B |
| 53 | 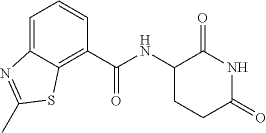 [SMILES: Cc1nc2cccc(C(=O)NC3CCC(=O)NC3=O)c2s1] | Synthetic Conditions B |
| 54 | 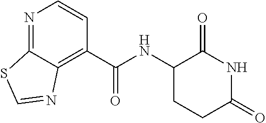 [SMILES: O=C1CCC(NC(=O)c2ccnc3scnc23)C(=O)N1] | Synthetic Conditions B |
| 55 | 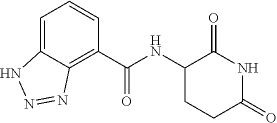 [SMILES: O=C1CCC(NC(=O)c2cccc3[nH]nnc23)C(=O)N1] | Synthetic Conditions C |
| 57 | 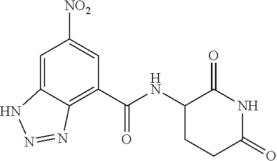 [SMILES: O=C1CCC(NC(=O)c2cc([N+](=O)[O-])cc3[nH]nnc23)C(=O)N1] | Synthetic Conditions C |
| 58 | 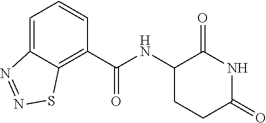 [SMILES: O=C1CCC(NC(=O)c2cccc3nnsc23)C(=O)N1] | Synthetic Conditions B |
| 62 | 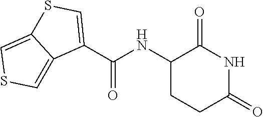 [SMILES: O=C1CCC(NC(=O)c2csc3cscc23)C(=O)N1] | Synthetic Conditions B |

### Example 148: Synthesis of N-(2,6-dioxopiperidin-3-yl)-1H-1,3-benzodiazole-7-carboxamide (2)

To a solution of 3-aminopiperidine-2,6-dione (0.96 g, 7.5 mmol) and N-hydroxybenzotriazole (1.22 g, 9.0 mmol) in DMF (15 mL) were added 1H-benzo[d]imidazole-7-carboxylic acid (8.25 g, 1.3 mmol), DMAP (37 mg, 0.30 mmol), and EDC (1.40 g, 9.0 mmol). The reaction mixture was stirred overnight at room temperature. Water (30 mL) was added and the obtained solution was extracted with dichloromethane (3×20 mL). The combined organic layers were washed with water, dried over Na₂SO₄, and concentrated under reduced pressure. The crude product was purified by preparative HPLC to obtain target compound (0.41 g, 20% yield).

¹H NMR: (400 MHz, DMSO-d₆) δ 10.49 (s, 1H), 9.67-9.52 (m, 1H), 9.45-9.28 (m, 1H), 8.12 (d, J=7.4 Hz, 1H) 8.01 (d, J=8.1 Hz, 1H), 7.64 (t, J=8.0 Hz, 1H), 4.90-4.78 (m, 1H), 3.85 (brs, 1H), 2.92-2.77 (m, 1H), 2.65-2.54 (m, 1H), 2.36-2.16 (m, 1H), 2.15-2.02 (m, 1H)

LCMS (m/z [M+H]⁺): 273.1

### Example 149: Synthesis of N-(2,6-dioxopiperidin-3-yl)-1-methyl-1H-benzo[d]imidazole-7-carboxamide (5)

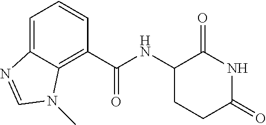 [SMILES: Cn1cnc2cccc(C(=O)NC3CCC(=O)NC3=O)c21]

This compound was synthesized using the general procedure shown in Reaction Scheme 1 and Synthetic Conditions B, above (4% yield), and 1-methyl-1H-benzo[d]imidazole-7-carboxylic acid (20 mg) as a starting material.

¹H NMR (500 MHz, DMSO) δ 10.87 (s, 1H), 8.94 (d, J=8.4 Hz, 1H), 8.29 (s, 1H), 7.84-7.71 (m, 1H), 7.37 (dt, J=7.4, 3.7 Hz, 1H), 7.28 (dd, J=8.0, 7.5 Hz, 1H), 4.80 (ddd, J=12.5, 8.4, 5.5 Hz, 1H), 3.87 (s, 3H), 2.83 (ddd, J=17.4, 13.1, 5.7 Hz, 1H), 2.56 (ddd, J=9.9, 5.2, 2.5 Hz, 1H), 2.15 (qd, J=12.9, 4.5 Hz, 1H), 2.07 (tdd, J=8.5, 5.6, 2.8 Hz, 1H).

LCMS (m/z [M+H]⁺): 286.7

### Example 150: Synthesis of N-(2,6-dioxopiperidin-3-yl)-5-hexanamido-1-methyl-1H-benzo[d]imidazole-7-carboxamide (6)

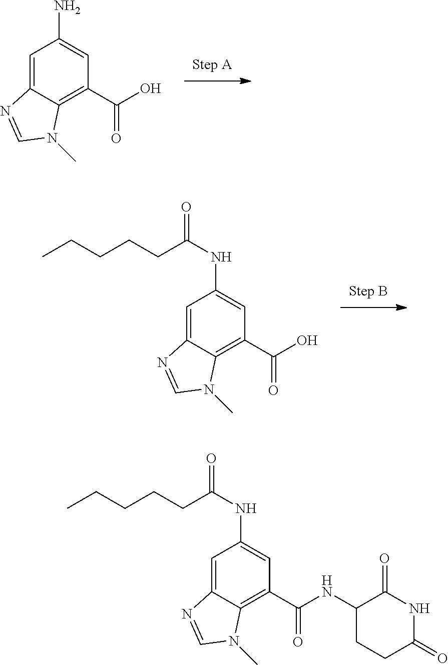 [SMILES: Cn1cnc2cc(N)cc(C(=O)O)c21 ; CCCCCC(=O)Nc1cc(C(=O)O)c2c(c1)ncn2C ; CCCCCC(=O)Nc1cc(C(=O)NC2CCC(=O)NC2=O)c2c(c1)ncn2C]

### Step A

5-amino-1-methyl-1H-benzo[d]imidazole-7-carboxylic acid dihydrochloride (20 mg, 0.076 mmol) and hexanoyl chloride (1.1eq.) were dissolved in 4 mL of dry DCM and cooled in water/ice bath. TEA (4 eq.) was slowly injected into the reaction mixture. The ice bath was removed and the reaction was allowed to warm up to ambient temperature. The reaction was completed in two hours, monitored by LCMS. The solution was diluted with DCM (10 mL) and washed with 7 mL 3% HCl water soln. The aqueous phase was then evaporated to yield off-white crystals and 5-hexanamido-1-methyl-1H-benzo[d]imidazole-7-carboxylic acid was used directly in the next step.

### Step B

This compound was synthesized using the general procedure shown in Reaction Scheme 1 and Synthetic Conditions B, above (29% yield), and 5-hexanamido-1-methyl-1H-benzo[d]imidazole-7-carboxylic acid (20 mg) as a starting material.

¹H NMR (500 MHz, DMSO) δ 10.87 (s, 1H), 10.00 (s, 1H), 8.97 (t, J=14.9 Hz, 1H), 8.21 (s, 1H), 8.16 (d, J=1.9 Hz, 1H), 7.51 (d, J=1.9 Hz, 1H), 4.79 (ddd, J=12.6, 8.4, 5.4 Hz, 1H), 3.82 (s, 3H), 2.82 (ddd, J=17.4, 13.1, 5.6 Hz, 1H), 2.57 (dt, J=16.6, 3.2 Hz, 1H), 2.31 (t, J=7.4 Hz, 2H), 2.20-2.09 (m, 1H), 2.09-2.01 (m, 1H), 1.67-1.56 (m, 2H), 1.37-1.25 (m, 4H), 0.87 (dt, J=7.1, 5.0 Hz, 3H).

LCMS (m/z [M+H]⁺): 400.2

### Example 151: Synthesis of N-(2,6-dioxopiperidin-3-yl)-2-methyl-1H-benzo[d]imidazole-4-carboxamide (15)

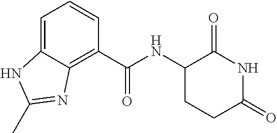 [SMILES: Cc1nc2c(C(=O)NC3CCC(=O)NC3=O)cccc2[nH]1]

This compound was synthesized using the general procedure shown in Reaction Scheme 1 and Synthetic Conditions B, above (20% yield), and 2-methyl-1H-benzo[d]imidazole-4-carboxylic acid (20 mg) as a starting material.

¹H NMR (500 MHz, DMSO) δ 12.73 (s, 1H), 10.90 (s, 1H), 10.29 (d, J=7.3 Hz, 1H), 7.82 (d, J=7.0 Hz, 1H), 7.63 (s, 1H), 7.32-7.23 (m, 1H), 4.87 (ddd, J=12.6, 7.1, 5.4 Hz, 1H), 2.89-2.76 (m, 1H), 2.58 (s, 3H), 2.55 (d, J=3.7 Hz, 1H), 2.28-2.19 (m, 1H), 2.18-2.07 (m, 1H).

LCMS (m/z [M+H]⁺): 286.5

### Example 152: Synthesis of 2-methyl-N-(2-oxoazepan-3-yl)-1H-1,3-benzodiazole-4-carboxamide (19)

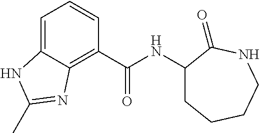 [SMILES: Cc1nc2c(C(=O)NC3CCCCNC3=O)cccc2[nH]1]

A vial was charged with 2-methyl-1H-1,3-benzodiazole-4-carboxylic acid (60.0 mg, 0.341 mmol, 1.000 eq), 3-aminoazepan-2-one hydrochloride (67.3 mg, 0.409 mmol, 1.200 eq), DMAP (4.2 mg, 0.034 mmol, 0.100 eq) and purged with Argon for 15 min. DMF (10 mL) added via syringe followed by DIPEA (0.119 mL, 0.681 mmol, 2.000 eq) and HATU (155.4 mg, 0.409 mmol, 1.200 eq) and the reaction mixture was stirred overnight. Solvent was evaporated under reduced pressure and the crude compound was purified by preparative TLC to provide 81 mg (82% yield) of the product.

¹H NMR (500 MHz, DMSO) δ 12.77 (s, 1H), 10.45 (s, 1H), 7.90-7.73 (m, 2H), 7.61 (dd, J=7.8, 0.7 Hz, 1H), 7.23 (t, J=7.8 Hz, 1H), 4.73 (ddd, J=10.9, 6.6, 1.3 Hz, 1H), 3.30-3.21 (m, 1H), 3.18-3.06 (m, 1H), 2.58 (s, 3H), 2.03-1.90 (m, 2H), 1.82-1.70 (m, 2H), 1.53 (dd, J=24.4, 11.9 Hz, 1H), 1.34-1.21 (m, 1H).

LCMS (m/z [M+H]⁺): 286.9

### Example 153: Synthesis of N-(2,7-dioxoazepan-3-yl)-2-methyl-1H-benzo[d]imidazole-4-carboxamide (20)

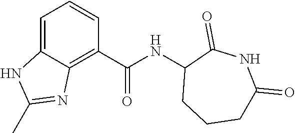 [SMILES: Cc1nc2c(C(=O)NC3CCCC(=O)NC3=O)cccc2[nH]1]

To a solution of 2-methyl-N-(2-oxoazepan-3-yl)-1H-1,3-benzodiazole-4-carboxamide (20.0 mg, 0.070 mmol, 1.000 eq) in MeCN (4.0 mL)/DMSO (0.085 mL)/water (0.010 mL) was added Dess-Martin periodinane (74.1 mg, 0.175 mmol, 2.500 eq). The suspension was heated at 80° C. for 1 h. Solvent was evaporated under reduced pressure and the crude product was purified by preparative TLC and HPLC to provide 16 mg (76%) of the product.

¹H NMR (500 MHz, DMSO) δ 12.73 (s, 1H), 10.67 (s, 1H), 10.38 (d, J=6.5 Hz, 1H), 7.81 (dd, J=7.6, 1.0 Hz, 1H), 7.64 (d, J=7.8 Hz, 1H), 7.27 (t, J=7.7 Hz, 1H), 5.19-5.06 (m, 1H), 3.08-2.95 (m, 1H), 2.65-2.61 (m, 1H), 2.60 (s, 3H), 2.35-2.22 (m, 1H), 2.08-1.94 (m, 1H), 1.89-1.69 (m, 2H).

LCMS (m/z [M+H]⁺): 301.1

### Example 154: Synthesis of 6-amino-N-(2,6-dioxopiperidin-3-yl)-2-(trifluoromethyl)-1H-1,3-benzodiazole-7-carboxamide (26)

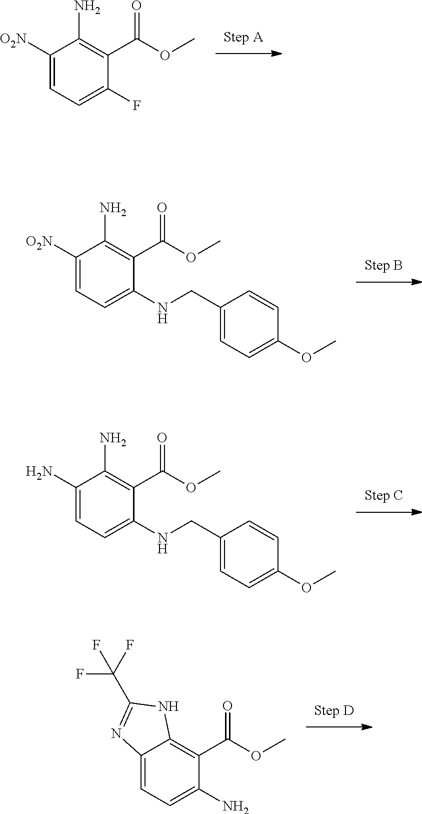 [SMILES: COC(=O)c1c(F)ccc([N+](=O)[O-])c1N ; COC(=O)c1c(NCc2ccc(OC)cc2)ccc([N+](=O)[O-])c1N ; COC(=O)c1c(NCc2ccc(OC)cc2)ccc(N)c1N ; COC(=O)c1c(N)ccc2nc(C(F)(F)F)[nH]c12]

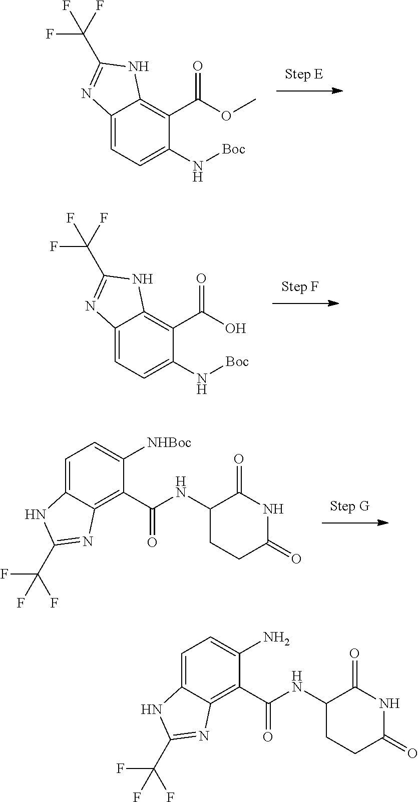 [SMILES: COC(=O)c1c(NC(=O)OC(C)(C)C)ccc2nc(C(F)(F)F)[nH]c12 ; CC(C)(C)OC(=O)Nc1ccc2nc(C(F)(F)F)[nH]c2c1C(=O)O ; CC(C)(C)OC(=O)Nc1ccc2[nH]c(C(F)(F)F)nc2c1C(=O)NC1CCC(=O)NC1=O ; Nc1ccc2[nH]c(C(F)(F)F)nc2c1C(=O)NC1CCC(=O)NC1=O]

### Step A

To a stirred solution of methyl 2-amino-6-fluoro-3-nitrobenzoate (2 g, 9.339 mmol) in DMSO (20 mL) was added K₂CO₃ (2.58 g, 18.67 mmol) followed by addition of (4-methoxyphenyl) methanamine (1.59 mL, 12.14 mmol). Then the reaction mixture was stirred at RT for 16 h. After completion of the reaction, quenched with ice water and precipitate was filtered and dried to give methyl 2-amino-6-((4-methoxybenzyl)amino)-3-nitrobenzoate 2.0 g (64% yield).

### Step B

To a stirred solution of methyl 2-amino-6-((4-methoxybenzyl)amino)-3-nitrobenzoate (550 mg, 1.66 mmol) in THE (16 ml) was added Zn (1.5 g, 21.6 mmol) followed by addition of NH₄Cl (1.15 g, 21.6 mmol) in water (3 ml) at 0° C. and stirred at RT for 1 h. After completion of the reaction, reaction mixture was filtered through celite, washed with ethyl acetate. Organic layer was washed with water, brine, dried over sodium sulphate and concentrated under reduced pressure to give methyl 2,3-diamino-6-((4-methoxybenzyl)amino)benzoate (250 mg, crude) as brownish solid.

### Step C

Methyl 2,3-diamino-6-((4-methoxybenzyl)amino)benzoate (2 g, 6.645 mmol) in TFA (20 mL) was stirred at rt for 16 h. After completion of the reaction, TFA was removed and quenched with aqueous NaHCO₃ and extracted with ethyl acetate. Organic layer washed with brine and dried over Na₂SO₄ and concentrated and purified by flash column chromatography to give methyl 6-amino-2-(trifluoromethyl)-1H-benzo[d]imidazole-7-carboxylate 200 mg (13% yield).

### Step D

To a stirred solution of methyl 6-amino-2-(trifluoromethyl)-1H-benzo[d]imidazole-7-carboxylate (600 mg, 2.317 mmol) in dioxane (5 mL) was added aq NaOH (1N) (15 mL) followed by addition of Boc₂O (3.2 mL, 13.9 mmol) at 0° C. and stirred at RT for 72 h. After completion of the reaction quenched with ice water and extracted with ethyl acetate, dried over sodium sulphate and concentrated. The crude product was purified by flash column chromatography to give methyl 6-((tert-butoxycarbonyl)amino)-2-(trifluoromethyl)-1H-benzo[d]imidazole-7-carboxylate 600 mg (72% yield).

### Step E

Solution of methyl 6-((tert-butoxycarbonyl)amino)-2-(trifluoromethyl)-1H-benzo[d]imidazole-7-carboxylate in 50% aq NaOH (13 mL) was stirred at 80° C. for 4 h. After completion of reaction, reaction mixture was acidified with 2M HCl and the precipitate was filtered to give 6-((tert-butoxycarbonyl)amino)-2-(trifluoromethyl)-1H-benzo[d]imidazole-7-carboxylic acid 300 mg (52% yield).

### Step F

tert-butyl N-{7-[(2,6-dioxopiperidin-3-yl)carbamoyl]-2-(trifluoromethyl)-1H-1,3-benzodiazol-6-yl}carbamate was synthesized using the general procedure shown in Reaction Scheme 1 and Synthetic Conditions B, above (36% yield) using 5-((tert-butoxycarbonyl)amino)-2-(trifluoromethyl)-1H-benzo[d]imidazole-4-carboxylic acid (30.0 mg) as a starting material.

### Step G

Tert-butyl (4-((2,6-dioxopiperidin-3-yl)carbamoyl)-2-(trifluoromethyl)-1H-benzo[d]imidazol-5-yl)carbamate (10.0 mg, 0.022 mmol, 1.000 eq) was dissolved in THE (0.220 mL) and 4M HCl in dioxane (0.038 mL, 1.098 mmol, 50.000 eq) was added. The mixture was stirring in RT for 4 h. Solvent was evaporated under reduced pressure to give 6-amino-N-(2,6-dioxopiperidin-3-yl)-2-(trifluoromethyl)-1H-1,3-benzodiazole-7-carboxamide hydrochloride 8.0 mg (88.0% yield).

1H NMR (500 MHz, DMSO) δ 14.15 (s, 1H), 10.91 (s, 1H), 10.19 (s, 1H), 7.54 (d, J=9.0 Hz, 1H), 6.94 (d, J=9.0 Hz, 1H), 4.86-4.77 (m, 1H), 2.88-2.75 (m, 1H), 2.63-2.54 (m, 1H), 2.33-2.22 (m, 1H), 2.10 (qd, J=12.9, 4.4 Hz, 1H).

LCMS (m/z [M+H]⁺): 356.3

### Example 155: Synthesis of 5-amino-N-(2,6-dioxopiperidin-3-yl)-2-(trifluoromethyl)-1H-benzo[d]imidazole-7-carboxamide (27)

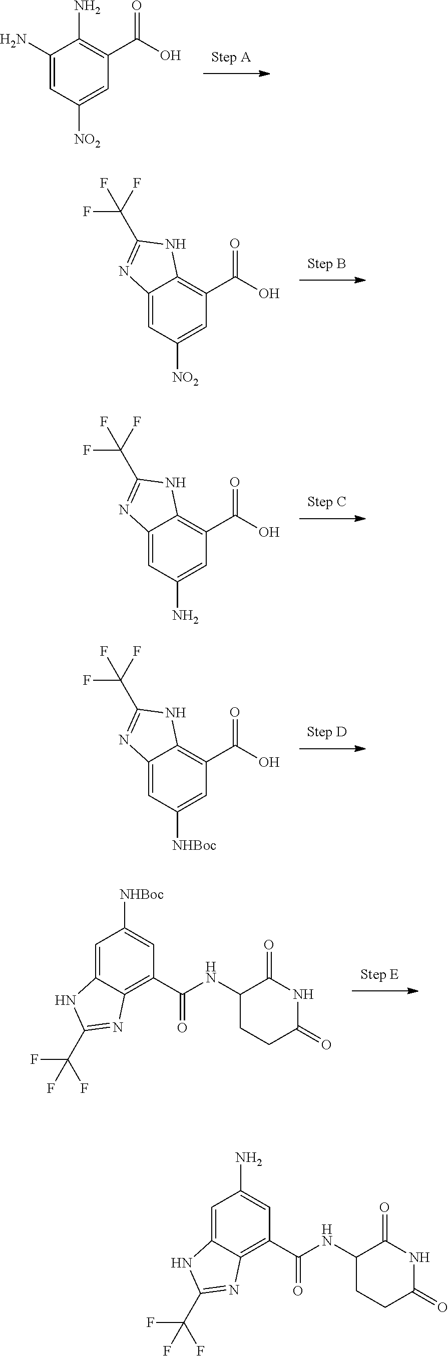 [SMILES: Nc1cc([N+](=O)[O-])cc(C(=O)O)c1N ; O=C(O)c1cc([N+](=O)[O-])cc2nc(C(F)(F)F)[nH]c12 ; Nc1cc(C(=O)O)c2[nH]c(C(F)(F)F)nc2c1 ; CC(C)(C)OC(=O)Nc1cc(C(=O)O)c2[nH]c(C(F)(F)F)nc2c1 ; CC(C)(C)OC(=O)Nc1cc(C(=O)NC2CCC(=O)NC2=O)c2nc(C(F)(F)F)[nH]c2c1 ; Nc1cc(C(=O)NC2CCC(=O)NC2=O)c2nc(C(F)(F)F)[nH]c2c1]

### Step A

TFA (2 mL) and 4(N) HCl (5 mL) were added to 2,3-diamino-5-nitrobenzoic acid (500 mg, 2.54 mmol). Then the resulting reaction mixture was allowed to reflux for 12 h. After completion of reaction, the reaction mixture was cooled to 0° C. and then carefully neutralized with 10M NaOH solution. Aqueous part was extracted by DCM (100 mL×3). Organic layer was washed with brine and dried over Na₂SO₄ and concentrated to get the crude. Finally the crude was triturated with pentane and ether to get crude compound of 5-nitro-2-(trifluoromethyl)-1H-benzo[d]imidazole-7-carboxylic acid (500 mg) as dark brown solid. Compound was used in next step without further purification

### Step B

To a stirred solution of 5-nitro-2-(trifluoromethyl)-1H-benzo[d]imidazole-7-carboxylic acid (500.0 mg, 1.82 mmol) in MeOH (10 mL) was added 10% Pd/C (193 mg). The reaction mixture was allowed to stir at rt for 4 h under hydrogen atmosphere. After completion of the reaction, the reaction mixture was filtered through celite and concentrated under reduced pressure to get methyl 5-amino-2-(trifluoromethyl)-1H-benzo[d]imidazole-7-carboxylic acid (500 mg) as crude which was used in next step without further purification.

### Step C

To an ice cooled solution of methyl 5-amino-2-(trifluoromethyl)-1H-benzo[d]imidazole-7-carboxylic acid (1.0 g, 4.1 mmol) in dioxane (5.0 mL) and H₂O (5.0 mL) was added TEA (0.85 mL, 6.1 mmol). The reaction mixture was allowed to stir at ice cool condition for 2-3 min. Boc₂O (1.0 mL, 4.49 mmol) was added and the reaction mixture was stirred at RT for 6 h. After completion of reaction, solvent was evaporated and the crude product was purified by preparative HPLC to give 5-((tert-butoxycarbonyl)amino)-2-(trifluoromethyl)-1H-benzo[d]imidazole-7-carboxylic acid (50 mg) as white solid (2.8% yield over 3 steps).

### Step D

Tert-butyl (7-((2,6-dioxopiperidin-3-yl)carbamoyl)-2-(trifluoromethyl)-1H-benzo[d]imidazol-5-yl)carbamate was synthesized using the general procedure shown in Reaction Scheme 1 and Synthetic Conditions B, above (37% yield) using 5-((tert-butoxycarbonyl)amino)-2-(trifluoromethyl)-1H-benzo[d]imidazole-7-carboxylic acid (30.0 mg) as a starting material.

### Step E

Tert-butyl (7-((2,6-dioxopiperidin-3-yl)carbamoyl)-2-(trifluoromethyl)-1H-benzo[d]imidazol-5-yl)carbamate (10.0 mg, 0.022 mmol, 1.000 eq) was dissolved in THE (0.220 mL) and 4 M HCl in dioxane_(0.038 mL, 1.098 mmol, 50.000 eq) was added. The mixture was stirring in RT for 4 h. Solvent was evaporated under reduced pressure to give 5-amino-N-(2,6-dioxopiperidin-3-yl)-2-(trifluoromethyl)-1H-benzo[d]imidazole-7-carboxamide hydrochloride.

¹H NMR (500 MHz, DMSO) δ 13.67 (s, 1H), 10.91 (s, 1H), 9.71 (s, 1H), 7.48-7.34 (m, 1H), 6.86 (d, J=2.1 Hz, 1H), 5.53 (s, 1H), 4.84 (ddd, J=12.4, 7.0, 5.2 Hz, 2H), 2.80 (ddd, J=17.3, 13.5, 5.5 Hz, 1H), 2.59-2.52 (m, 1H), 2.32-2.21 (m, 1H), 2.15-2.03 (m, 1H).

LCMS (m/z [M+H]⁺): 355.9

### Example 156: Synthesis of 7-amino-N-(2,6-dioxopiperidin-3-yl)-2-(trifluoromethyl)-1H-benzo[d]imidazole-4-carboxamide (28)

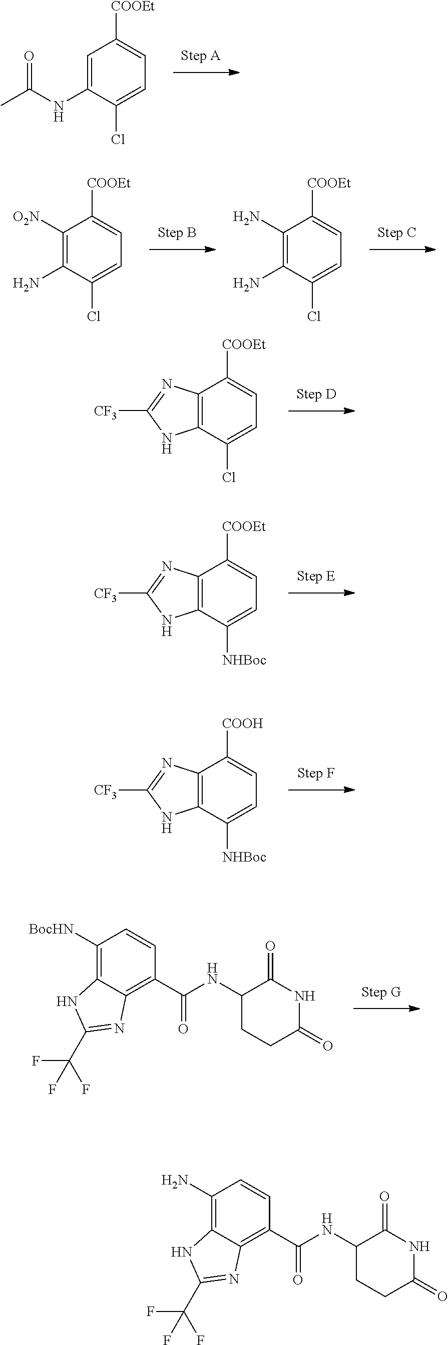

### Step A

To ethyl 3-acetamido-4-chlorobenzoate (20.0 g, 82.97 mmol) was dropwise added 40.0 mL of 100% HNO₃ at −15° C. and the resultant reaction mixture was stirred and warmed up slowly to 10° C. during 2 h and then stirred at RT for 12 h, poured into crashed ice, the solids were filtered, dried under reduced pressure and the mixture of nitro compounds (16 g) was used directly in the next step.

To a stirred solution of nitro compounds in 160 mL of ethanol was added 7.5 mL of conc. H₂SO₄. The reaction mixture was refluxed for 16 h, concentrated under reduced pressure and ice-cold water was added. The product was extracted into DCM, the combined organic layers were washed with brine, dried over Na₂SO₄ and concentrated. The crude product was purified by flash column chromatography to give ethyl 3-amino-4-chloro-2-nitrobenzoate (6.3 g, 30%).

### Step B

To a stirred solution of ethyl 3-amino-4-chloro-2-nitrobenzoate (6.3 g, 25.753 mmol) in ethanol (60.0 mL) and water (30.0 mL) was added Fe powder (10.78 g) followed by NH₄Cl (1.791 g). The reaction mixture was refluxed for 12 h, concentrated under reduced pressure, diluted with DCM, filtered through celite bed and concentrated under reduced pressure. The crude product was purified by flash column chromatography to give ethyl 2,3-diamino-4-chlorobenzoate (5 g, 90.45%).

### Step C

To ethyl 2,3-diamino-4-chlorobenzoate (2.0 g, 9.317 mmol, 1.0 eq) was added 15 ml of TFA and the reaction mixture was refluxed for 12 h and concentrated under reduced pressure. To the residue was added NaHCO₃ solution and the product was extracted with ethyl acetate, washed with brine, dried over Na₂SO₄ and concentrated. The crude product was purified by flash column chromatography to give ethyl 7-chloro-2-(trifluoromethyl)-1H-benzo[d]imidazole-4-carboxylate (2.4 g, 88% yield).

### Step D

A solution of ethyl 7-chloro-2-(trifluoromethyl)-1H-benzo[d]imidazole-4-carboxylate (1.0 g, 3.417 mmol) in dioxane (12 mL) was degassed under argon atmosphere for 10-15 min. Cs₂CO₃ (2.22 g, 6.834 mmol), NH₂Boc (1.60 g, 13.669 mmol), X-phos (326 mg, 0.683 mmol) and X-phosPdG3 (0.289 g, 0.342 mmol) were added and reaction mixture was stirred at 85° C. for 16 h. Reaction mixture was filtered through celite bed, concentrated and purified by flash column chromatography to give ethyl 7-((tert-butoxycarbonyl)amino)-2-(trifluoromethyl)-1H-benzo[d]imidazole-4-carboxylate (800 mg, 62% yield).

### Step E

A stirred solution of ethyl 7-((tert-butoxycarbonyl)amino)-2-(trifluoromethyl)-1H-benzo[d]imidazole-4-carboxylate (500.0 mg, 1.339 mmol) in MeOH (3.0 mL) and THE (3.0 mL) was added slowly 50% aqueous NaOH solution (6.0 mL) at ice cool condition. Then the resultant reaction mixture was allowed to stir at rt for 16 h. Reaction mixture was concentrated under reduced pressure and then it was diluted with water and washed with ethyl acetate. After that the aqueous part was gently neutralized with saturated aqueous citric acid solution in ice cool condition and then it was extracted with ethyl acetate. Then the combined organic layer was washed with brine and then dried over Na₂SO₄, filtered and concentrated to get the crude which was triturated with pentane and ether to get 7-((tert-butoxycarbonyl)amino)-2-(trifluoromethyl)-1H-benzo[d]imidazole-4-carboxylic acid (250 mg, 54.06% yield) as white solid.

### Step F

Tert-butyl (4-((2,6-dioxopiperidin-3-yl)carbamoyl)-2-(trifluoromethyl)-1H-benzo[d]imidazol-7-yl)carbamate was synthesized using the general procedure shown in Reaction Scheme 1 and Synthetic Conditions B, above (80% yield), and 7-((tert-butoxycarbonyl)amino)-2-(trifluoromethyl)-1H-benzo[d]imidazole-4-carboxylic acid (30 mg) as a starting material.

1H NMR (500 MHz, DMSO) δ 14.02 (s, 1H), 10.93 (s, 1H), 9.57 (s, 1H), 8.93 (s, 1H), 7.98 (s, 2H), 4.86 (dt, J=12.3, 5.9 Hz, 1H), 2.88-2.79 (m, 1H), 2.57 (s, 1H), 2.29 (d, J=12.4 Hz, 1H), 2.11 (td, J=13.1, 4.5 Hz, 1H), 1.53 (s, 9H).

LCMS (m/z [M+H]⁺): 456.5

### Step G

To the mixture of tert-butyl (4-((2,6-dioxopiperidin-3-yl)carbamoyl)-2-(trifluoromethyl)-1H-benzo[d]imidazol-7-yl)carbamate (8 mg, 0.018 mmol) in DCM (0.5 mL) was added TFA (0.1 mL) and the reaction mixture was stirred at RT for 18 h. The mixture was concentrated under reduced pressure and was purified by HPLC to give 7-amino-N-(2,6-dioxopiperidin-3-yl)-2-(trifluoromethyl)-1H-1,3-benzodiazole-4-carboxamide trifluoroacetate (44% yield).

¹H NMR (500 MHz, DMSO) δ 10.51 (s, 1H), 7.75 (d, J=8.3 Hz, 1H), 6.58 (s, 1H), 5.97 (d, J=72.1 Hz, 2H), 4.76 (d, J=10.7 Hz, 1H), 2.81-2.73 (m, 1H), 2.60 (dd, J=17.5, 3.9 Hz, 1H), 2.12 (d, J=26.4 Hz, 2H).

LCMS (m/z [M+H]⁺): 356.0

### Example 157: Synthesis of 6-(aminomethyl)-N-(2,6-dioxopiperidin-3-yl)-2-methyl-1H-benzo[d]imidazole-4-carboxamide (31)

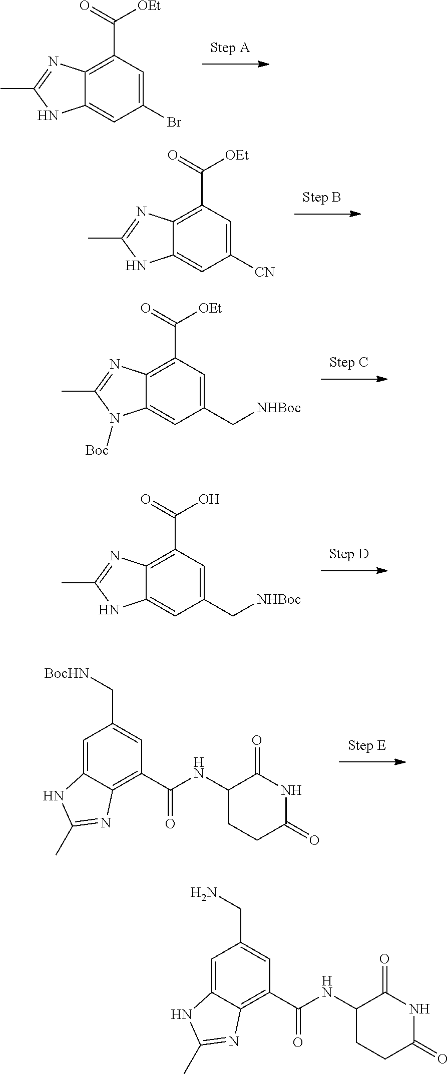

### Step A

To a degassed solution of ethyl 6-bromo-2-methyl-1H-benzo[d]imidazole-4-carboxylate (500 mg, 1.76 mmol) in DMF (12 mL) were added ZN(CN)₂ (518 mg, 4.41 mmol) and Pd(PPh₃)₄ (408 mg, 0.35 mmol) and the reaction mixture was at 120° C. for 16 h, quenched with ice water, extracted with ethyl acetate, dried over Na₂SO₄, concentrated under reduced pressure and purified by flash column chromatography to give ethyl 6-cyano-2-methyl-1H-benzo[d]imidazole-4-carboxylate (27% yield).

### Step B

To a solution of ethyl 6-cyano-2-methyl-1H-benzo[d]imidazole-4-carboxylate (400 mg, 1.747 mmol) in ethanol (13 ml) were added Raney-nickel and Boc₂O (2.1 ml, 8.734 mmol) and the reaction mixture was stirred under hydrogen (15 psi) for 16 h, filtered through celite bed, filtrates were concentrated under reduced pressure and purified by flash column chromatography to give 1-(tert-butyl) 4-ethyl 6-(((tert-butoxycarbonyl)amino)methyl)-2-methyl-1H-benzo[d]imidazole-1,4-dicarboxylate (47% yield).

### Step C

To a solution of 1-(tert-butyl) 4-ethyl 6-(((tert-butoxycarbonyl)amino)methyl)-2-methyl-1H-benzo[d]imidazole-1,4-dicarboxylate (430 mg, 0.993 mmol) in THF:MeOH 1:1 (10 mL) was added 50% aqueous NaOH (4 mL) and the reaction mixture was stirred at RT for 16 h, neutralized with 1M HCl, and filtered. The solids were dried to give 6-(((tert-butoxycarbonyl)amino)methyl)-2-methyl-1H-benzo[d]imidazole-4-carboxylic acid (62% yield).

### Step D

Tert-butyl ((4-((2,6-dioxopiperidin-3-yl)carbamoyl)-2-methyl-1H-benzo[d]imidazol-6-yl)methyl)carbamate was synthesized using the general procedure shown in Reaction Scheme 1 and Synthetic Conditions B, above (45% yield), and 6-(((tert-butoxycarbonyl)amino)methyl)-2-methyl-1H-benzo[d]imidazole-4-carboxylic acid (30 mg) as a starting material.

¹H NMR (500 MHz, DMSO) δ 12.64 (s, 1H), 10.89 (s, 1H), 10.24 (d, J=7.3 Hz, 1H), 8.16 (s, 1H), 7.74 (s, 1H), 7.49 (s, 1H), 7.45 (t, J=6.4 Hz, 1H), 4.88 (dt, J=12.6, 6.4 Hz, 1H), 4.24 (d, J=6.2 Hz, 2H), 2.82 (ddd, J=17.3, 13.3, 5.5 Hz, 1H), 2.61-2.52 (m, 4H), 2.27-2.20 (m, 1H), 2.11 (qd, J=12.9, 4.3 Hz, 1H), 1.40 (s, 9H).

LCMS (m/z [M+H]⁺): 416.0

### Step E

Tert-butyl ((4-((2,6-dioxopiperidin-3-yl)carbamoyl)-2-methyl-1H-benzo[d]imidazol-6-yl)methyl)carbamate was suspended in DCM (0.5 mL). To the mixture was added TFA (0.1 mL) and stirred for 2 h at RT. The crude was concentrated in vacuo, dissolved in water and freeze-dried to give 6-(aminomethyl)-N-(2,6-dioxopiperidin-3-yl)-2-methyl-1H-benzo[d]imidazole-4-carboxamide.

¹H NMR (500 MHz, DMSO) δ 10.93 (s, 1H), 10.12 (s, 1H), 8.14 (s, 3H), 7.97 (d, J=1.6 Hz, 1H), 7.79 (s, 1H), 4.88 (dt, J=13.0, 7.1 Hz, 1H), 4.20 (q, J=5.8 Hz, 2H), 2.84 (ddd, J=17.3, 13.0, 6.0 Hz, 1H), 2.67-2.53 (m, 4H), 2.25-2.09 (m, 2H).

LCMS (m/z [M+H]⁺): 315.8

### Example 158: Synthesis of 7-(aminomethyl)-N-(2,6-dioxopiperidin-3-yl)-2-methyl-1H-benzo[d]imidazole-4-carboxamide (32)

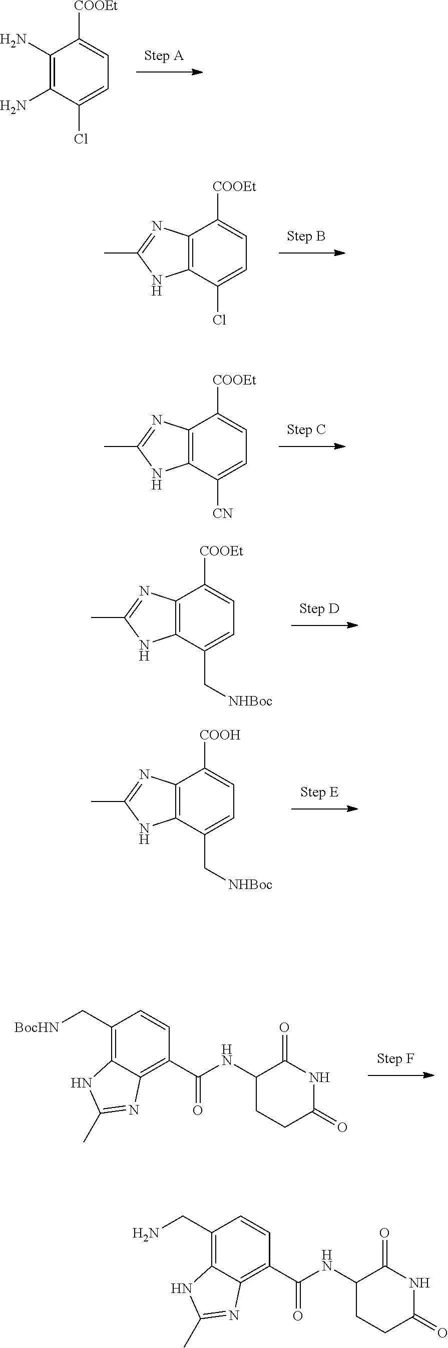

### Step A

To a stirred solution of ethyl 2,3-diamino-4-chlorobenzoate (1.5 g, 6.99 mmol) in toluene (20.0 mL) was added respectively triethyl orthoacetate (5.1 mL, 27.95 mmol) and PTSA (0.337 g, 1.957 mmol) and the reaction mixture was refluxed for 16 h, concentrated under reduced pressure and the crude product was purified by flash column chromatography to give ethyl 7-chloro-2-methyl-1H-benzo[d]imidazole-4-carboxylate 1.2 g (71% yield).

### Step B

A solution of ethyl 7-chloro-2-methyl-1H-benzo[d]imidazole-4-carboxylate (400 mg, 1.676 mmol) in DMF (10 mL) was degassed under argon atmosphere for 10-15 minutes. Zn(CN)₂ (492 mg, 4.19 mmol), X-phos (159.792 mg, 0.335 mmol) and X-phosPdG3 (0141.86 mg, 0.168 mmol) were added and the reaction mixture was heated to 110° C. for 16 h. The mixture was filtered through celite bed, diluted with water, the product was extracted with ethyl acetate, washed with brine, dried over Na₂SO₄ and concentrated under reduced pressure. The crude product was purified by flash column chromatography to give ethyl 7-cyano-2-methyl-1H-benzo[d]imidazole-4-carboxylate 251 mg (65% yield).

### Step C

The a stirred solution of ethyl 7-cyano-2-methyl-1H-benzo[d]imidazole-4-carboxylate (3) (375 mg, 1.636 mmol) in ethanol (10 mL) was added Boc₂O (0.564 mL, 2.454 mmol) and Raney-nickel (200 mg) and reaction mixture was stirred at RT under hydrogen atmosphere for 16 h, filtered through celite bed and concentrated under reduced pressure. The crude product was purified by flash column chromatography to give ethyl 7-(((tert-butoxycarbonyl)amino)methyl)-2-methyl-1H-benzo[d]imidazole-4-carboxylate 230 mg (42% yield).

### Step D

To a solution of ethyl 7-(((tert-butoxycarbonyl)amino)methyl)-2-methyl-1H-benzo[d]imidazole-4-carboxylate (200.0 mg, 0.6 mmol) in MeOH (1 mL) and THE (1 mL) was added 50% NaOH solution (2 mL) at 0° C. The reaction mixture was stirred at RTfor 16 h, concentrated under reduced pressure, diluted with water and washed with DCM. The aqueous phase was gently acidified by citric acid solution and the product was extracted with ethyl acetate, washed with brine, dried over Na₂SO₄ and concentrated under reduced pressure. The crude product was triturated with diethyl ether to give 7-(((tert-butoxycarbonyl)amino)methyl)-2-methyl-1H-benzo[d]imidazole-4-carboxylic acid 60 mg (32%).

### Step E

Tert-butyl ((4-((2,6-dioxopiperidin-3-yl)carbamoyl)-2-methyl-1H-benzo[d]imidazol-7-yl)methyl)carbamate was synthesized using the general procedure shown in Reaction Scheme 1 and Synthetic Conditions B, above (47% yield), and 7-(((tert-butoxycarbonyl)amino)methyl)-2-methyl-1H-benzo[d]imidazole-4-carboxylic acid (20 mg) as a starting material.

¹H NMR (500 MHz, DMSO) δ 12.66 (s, 1H), 10.89 (s, 1H), 10.24 (d, J=7.3 Hz, 1H), 8.15 (s, 1H), 7.79 (d, J=7.8 Hz, 1H), 7.47 (t, J=6.1 Hz, 1H), 7.13 (d, J=7.9 Hz, 1H), 4.86 (ddd, J=12.5, 7.2, 5.2 Hz, 1H), 4.42 (d, J=6.1 Hz, 2H), 2.81 (ddd, J=17.3, 13.5, 5.5 Hz, 1H), 2.61-2.51 (m, 4H), 2.26-2.20 (m, 1H), 2.16-2.07 (m, 1H), 1.40 (s, 9H).

LCMS (m/z [M+H]⁺): 416.0

### Step F

Tert-butyl ((4-((2,6-dioxopiperidin-3-yl)carbamoyl)-2-methyl-1H-benzo[d]imidazol-7-yl)methyl)carbamate was suspended in DCM (0.5 mL). To the mixture was added TFA (0.1 mL) and stirred for 2 h at RT. The crude was concentrated in vacuo, dissolved in water and freeze-dried to give 7-(aminomethyl)-N-(2,6-dioxopiperidin-3-yl)-2-methyl-1H-benzo[d]imidazole-4-carboxamide.

¹H NMR (500 MHz, DMSO) δ10.91 (s, 1H), 10.12 (s, 1H), 9.20 (s, 1H), 8.30 (s, 3H), 7.85 (d, J=7.8 Hz, 1H), 7.37 (d, J=7.9 Hz, 1H), 4.82 (d, J=10.7 Hz, 1H), 4.39 (d, J=5.7 Hz, 2H), 2.88-2.77 (m, 1H), 2.64 (s, 3H), 2.62-2.50 (m, 1H), 2.17 (s, 2H).

LCMS (m/z [M+H]⁺): 316.1

### Example 159: Synthesis of 5-(2,4-dimethoxyphenyl)-N-(2,6-dioxopiperidin-3-yl)-2-methyl-3H-imidazo[4,5-b]pyridine-7-carboxamide (33)

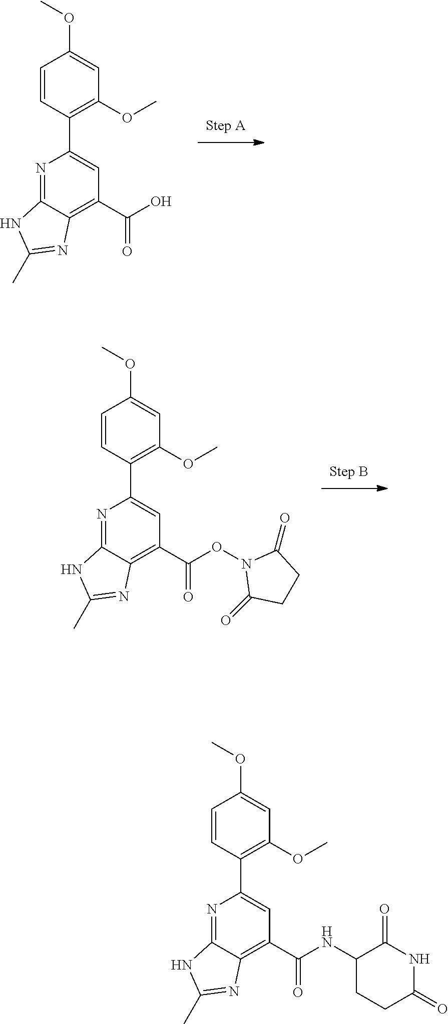 [SMILES: COc1ccc(-c2cc(C(=O)O)c3nc(C)[nH]c3n2)c(OC)c1 ; COc1ccc(-c2cc(C(=O)ON3C(=O)CCC3=O)c3nc(C)[nH]c3n2)c(OC)c1 ; COc1ccc(-c2cc(C(=O)NC3CCC(=O)NC3=O)c3nc(C)[nH]c3n2)c(OC)c1]

### Step A

To a suspension of 5-(2,4-dimethoxyphenyl)-2-methyl-1H-imidazo[4,5-b]pyridine-7-carboxylic acid (10.0 mg, 31.917 μmol, 1.000 eq) and HOSu (4.4 mg, 38.300 μmol, 1.200 eq) in DCM (1.0 mL) was added a solution of DCC (7.9 mg, 38.300 μmol, 1.200 eq) in DCM (0.500 mL). The reaction mixture was stirred at RT for 18 h. The reaction mixture was concentrated under reduced pressure and purified by preparative TLC to give 2,5-dioxopyrrolidin-1-yl 5-(2,4-dimethoxyphenyl)-2-methyl-1H-imidazo[4,5-b]pyridine-7-carboxylate (71% yield).

### Step B

To a solution of 3-aminopiperidine-2,6-dione hydrochloride (8.4 mg, 51.171 μmol, 3.000 eq) and DIPEA (9 μL, 51.171 μmol, 3.000 eq) in DMF (2.0 mL) was added 2,5-dioxopyrrolidin-1-yl 5-(2,4-dimethoxyphenyl)-2-methyl-1H-imidazo[4,5-b]pyridine-7-carboxylate (7.0 mg, 17.057 μmol, 1.000 eq) in one portion. The reaction mixture was stirred at RT for 18 h. The solvent was evaporated under reduced pressure and the residue was purified by preparative TLC to provide 4.1 mg (56%) of product.

¹H NMR (500 MHz, DMSO) δ 13.04 (s, 1H), 10.54 (s, 1H), 8.72 (d, J=8.0 Hz, 1H), 7.76 (d, J=8.5 Hz, 1H), 7.65 (s, 1H), 6.73 (d, J=2.4 Hz, 1H), 6.70 (dd, J=8.6, 2.4 Hz, 1H), 4.81 (q, J=8.2 Hz, 1H), 3.87 (s, 3H), 3.87 (s, 3H), 2.81 (dt, J=18.0, 9.5 Hz, 1H), 2.67-2.57 (m, 1H), 2.53 (s, 3H), 2.15 (dq, J=9.1, 5.2, 4.1 Hz, 2H).

LCMS (m/z [M+H]⁺): 423.9

### Example 160: Synthesis of N-(2,6-dioxopiperidin-3-yl)-2-methyl-1H-thieno[2,3-d]imidazole-6-carboxamide (59)

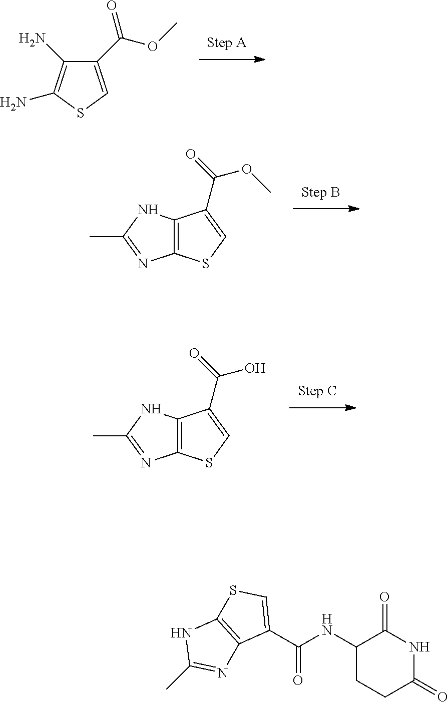

### Step A

A mixture of methyl 4,5-diaminothiophene-3-carboxylate (400 mg, 2.04 mmol) in dioxane (3 mL), triethyl orthoacetate (3 mL) and PTSA (102 mg, 0.40 mmol) was heated to reflux for 16 h, the reaction mixture was concentrated under reduced pressure and the crude material was purified by flash column chromatography to give methyl 2-methyl-1H-thieno[2,3-d]imidazole-6-carboxylate 200 mg (50% yield).

### Step B

To a stirred solution of methyl 2-methyl-1H-thieno[2,3-d]imidazole-6-carboxylate (0.13 g, 1.02 mmol) in methanol (0.5 mL) and THE (2 mL) was added NaOH (27 mg, 0.68 mmol) in water (0.5 mL) and the resulting solution was stirred at RT for 16 h. The reaction mixture was diluted with water and washed with ethyl acetate. The aqueous part was acidified with 6N HCl to pH˜5 and the resulting precipitate was filtered, washed with water and purified by HPLC to give 2-methyl-1H-thieno[2,3-d]imidazole-6-carboxylic acid 70 mg (37%).

### Step C

N-(2,6-dioxopiperidin-3-yl)-2-methyl-1H-thieno[2,3-d]imidazole-6-carboxamide was synthesized using the general procedure shown in Reaction Scheme 1 and Synthetic Conditions B, above (17% yield), and 2-methyl-3H-thieno[2,3-d]imidazole-6-carboxylic acid (20 mg) as a starting material.

¹H NMR (500 MHz, DMSO): δ 12.53 (s, 1H), 10.87 (s, 1H), 8.70 (d, J=8.0 Hz, 1H), 7.82 (s, 1H), 4.78-4.67 (m, 1H), 2.81 (ddd, J=17.4, 13.3, 5.5 Hz, 1H), 2.56 (ddd, J=17.1, 4.1, 2.9 Hz, 1H), 2.43 (s, 3H), 2.16 (qd, J=12.9, 4.5 Hz, 1H), 2.04-1.96 (m, 1H).

LCMS (m/z [M+H]⁺): 293.0

### Example 161: Synthesis of N-(2,6-dioxopiperidin-3-yl)-1H-thieno[2,3-d]imidazole-6-carboxamide 60)

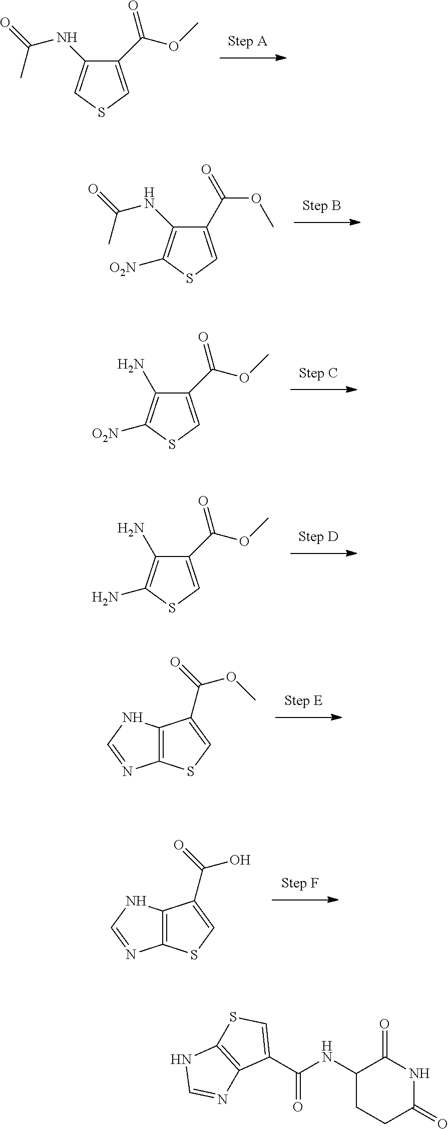

### Step A

A Solution of methyl 4-acetamidothiophene-3-carboxylate (3 g, 12.3 mmol) in acetic anhydride (40 mL) was cooled at −15° C. To it a precooled solution (at −15° C.) of concentrated nitric acid (6 mL) in 30 mL acetic anhydride was added drop wise very slowly with stirring. After 30 min the reaction mixture was poured into crushed ice and the resulting light yellow coloured solid was filtered. The solid was thoroughly washed with water and diethyl ether to give 2.4 g (81%) of methyl 4-acetamido-5-nitrothiophene-3-carboxylate.

### Step B

To a stirred solution of methyl 4-acetamido-5-nitrothiophene-3-carboxylate (2 g, 8.19 mmol) in 4N HCl-dioxane (20 mL), methanol (10 mL) was added and the resulting solution was heated at 100° C. for 16 h. After cooling, dioxane was removed under reduced pressure. The residue was diluted with water and extracted with ethyl acetate. The organic layer was washed with saturated sodium bicarbonate and brine and dried over Na₂SO₄. After concentration under reduced pressure, the crude methyl 4-amino-5-nitrothiophene-3-carboxylate 850 mg (51%) was used in the next step without further purification.

### Step C

To a stirred solution of methyl 4-amino-5-nitrothiophene-3-carboxylate (1 g, 4.95 mmol) in a mixture of dioxane-HCl (10 mL) and methanol (10 mL), SnCl₂ was added and the resulting solution was stirred at RT for 2 h. The reaction mixture was then poured on to a precooled solution of ammonium hydroxide and extracted with ethyl acetate. The organic layer was dried over anhydrous sodium sulfate, filtered and dried under reduced pressure. The crude methyl 4,5-diamino-thiophene-3-carboxylate 700 mg (82%) was used in the next step without further purification.

### Step D

To a stirred solution of methyl 4,5-diaminothiophene-3-carboxylate (650 mg, 3.78 mmol) in a mixture of trimethyl orthoformate (2.5 mL) and toluene (2.5 mL), a catalytic amount of PTSA (189 mg, 0.75 mmol) was added and the resulting solution was heated at 110° C. for 2 h. After that the volatiles were removed under reduced pressure, the crude material was purified by flash column chromatography to give 350 mg (50%) of methyl 1H-thieno[2,3-d]imidazole-6-carboxylate.

### Step E

To a stirred solution of methyl 1H-thieno[2,3-d]imidazole-6-carboxylate (400 mg, 2.2 mmol mmol) in methanol (3 mL) and THE (3 mL), NaOH (439 mg, 10.9 mmol) dissolved in water (1 mL) was added and the resulting solution was stirred for 16 h. The reaction mixture was diluted with water and washed with ethyl acetate. The aqueous part was acidified with 6N HCl to pH˜5 and the resulting brown coloured precipitate was filtered, washed with water and diethyl ether to obtain 1H-thieno[2,3-d]imidazole-6-carboxylic acid 230 mg (62%).

### Step F

N-(2,6-dioxopiperidin-3-yl)-1H-thieno[2,3-d]imidazole-6-carboxamide was synthesized using the general procedure shown in Reaction Scheme 1 and Synthetic Conditions B, above (40% yield), and 1H-thieno[2,3-d]imidazole-6-carboxylic acid (20 mg) as a starting material.

¹H NMR (500 MHz, DMSO) δ 12.79 (s, 1H), 10.88 (s, 1H), 8.74 (d, J=8.2 Hz, 1H), 7.99 (d, J=1.3 Hz, 1H), 7.90 (s, 1H), 4.74 (ddd, J=13.3, 8.1, 5.3 Hz, 1H), 2.81 (ddd, J=17.2, 13.3, 5.5 Hz, 1H), 2.57 (dt, J=18.0, 4.1 Hz, 1H), 2.16 (qd, J=12.9, 4.5 Hz, 1H), 2.01 (dtd, J=13.1, 5.4, 2.8 Hz, 1H).

LCMS (m/z [M+H]⁺): 279.0

### Example 162: Synthesis of N-(2,6-dioxopiperidin-3-yl)-2,5,6-trimethyl-4H-thieno[3,2-b]pyrrole-3-carboxamide (61)

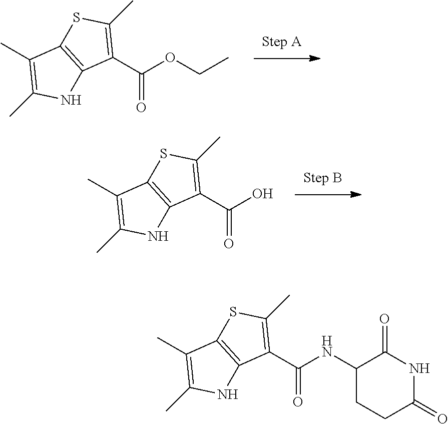 [SMILES: CCOC(=O)c1c(C)sc2c(C)c(C)[nH]c12 ; Cc1[nH]c2c(C(=O)O)c(C)sc2c1C ; Cc1[nH]c2c(C(=O)NC3CCC(=O)NC3=O)c(C)sc2c1C]

### Step A

To a solution of ethyl 2,5,6-trimethyl-4H-thieno[3,2-b]-pyrrole-3-carboxylate (10.0 mg, 0.042 mmol, 1.000 eq) in a mixture of H₂O (1.0 mL), THE (1.0 mL) and MeOH (1.0 mL) was added 1M LiOH (2.0 mL, 2.000 mmol, 17.702 eq). The reaction was stirred for 24 h at rt. After this time, to a mixture was added 1M HCl (2.0 mL, 2.000 mmol, 17.702 eq) to neutralize pH. The crude was concentrated in vacuo and used to the next step without further purification.

### Step B

N-(2,6-dioxopiperidin-3-yl)-2,5,6-trimethyl-4H-thieno[3,2-b]pyrrole-3-carboxamide was synthesized using the general procedure shown in Reaction Scheme 1 and Synthetic Conditions B, above (23% yield), and 2,5,6-trimethyl-4H-thieno[3,2-b]pyrrole-3-carboxylic acid (8.8 mg) as a starting material.

1H NMR (500 MHz, DMSO) δ 10.87 (s, 1H), 10.45 (s, 1H), 7.94 (d, J=8.2 Hz, 1H), 4.76 (ddd, J=12.3, 8.2, 5.4 Hz, 1H), 2.80 (ddd, J=17.3, 13.4, 5.6 Hz, 1H), 2.63 (s, 3H), 2.59-2.52 (m, 1H), 2.22 (s, 3H), 2.16 (qd, J=13.0, 4.5 Hz, 1H), 2.05 (qd, J=4.8, 2.3 Hz, 1H), 2.02 (s, 3H).

LCMS (m/z [M+H]⁺): 319.8

### Synthesis of Ligase Ligand Moieties—Compounds of Formula (IV)

### Example 163: Synthesis of 3-(5-amino-2-methylquinolin-3-yl)piperidine-2,6-dione (63)

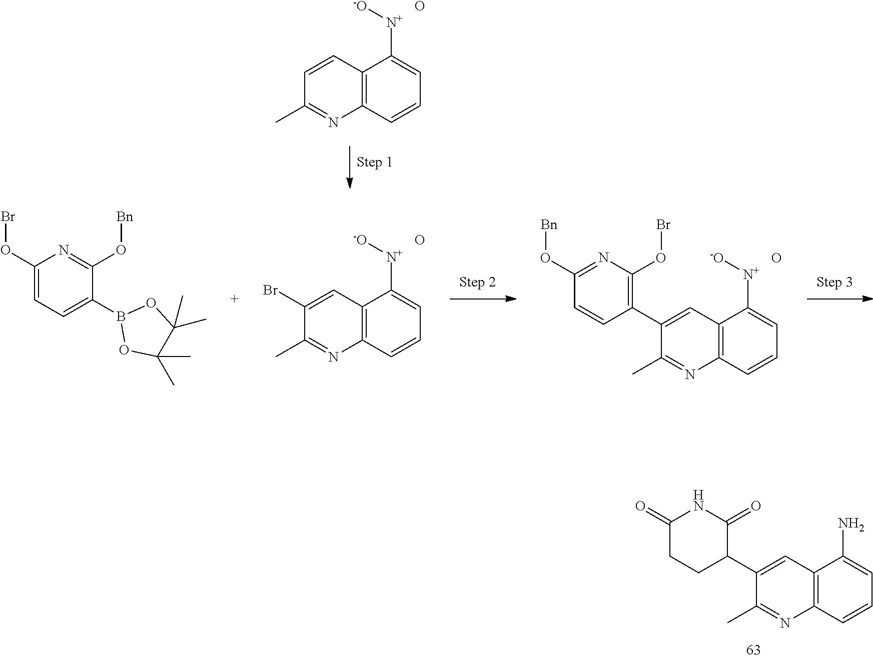

### Step 1: Synthesis of 3-bromo-2-methyl-5-nitro-8,8a-dihydroquinoline

2-Methyl-5-nitro-8,8a-dihydroquinoline (19.8 g, 105.3 mmol) was dissolved in dichloromethane (250 mL) and cooled to 5° C. in an ice bath. m-CPBA (32.9 g, 133.4 mmol, 70%) was added in portions thereto and the reaction mixture was stirred at room temperature (20-25° C.) for 12 hrs. The mixture was washed with 2M NaOH solution (2×150 mL), dried over anhydrous sodium sulfate, and evaporated under vacuum to afford a yellow solid (22 g). The solid was dissolved in CHCl₃ (200 mL), the obtained solution was cooled to 5° C. in the ice-bath, and phosphoryl bromide (62.6 g, 218.3 mmol) in CHCl₃ (300 mL) was added dropwise to the reaction mixture. The mixture was stirred at room temperature (20-25° C.) for 12 hrs, poured into ice-water, basified to pH=12 with solid potassium carbonate, and extracted with CHCl₃ (3×100 mL). The combined extracts were dried over anhydrous sodium sulfate and evaporated under vacuum. The crude product was purified by flash column chromatography (eluent Hexane-MTBE 0-100%) to afford 2.9 g of 3-bromo-2-methyl-5-nitro-8,8a-dihydroquinoline (10% yield) as a brown solid.

### Step 2: Synthesis of 3-[2,6-bis(benzyloxy)pyridin-3-yl]-2-methyl-5-nitro-8,8a-dihydroquinoline

2,6-Bis(benzyloxy)-3-(4,4,5,5-tetramethyl-1,3,2-dioxaborolan-2-yl)pyridine (4.55 μg, 10.9 mmol), tripotassium phosphate (4.8 g, 22.6 mmol), and Pd(dppf)Cl₂ CH₂Cl₂ (0.86 g, 1 mmol) were added sequentially to a solution of 3-bromo-2-methyl-5-nitro-8,8a-dihydroquinoline (2.9 g, 10.86 mmol) in 1,4-dioxane (50 mL) and water (5 mL). The obtained mixture was stirred at 100° C. for 12 hrs under an argon atmosphere. The solvents were removed under vacuum, the residue was diluted with EtOAc (100 mL) and filtered through a pad of silica gel. The filtrate was evaporated under vacuum and recrystallized from EtOAc to afford 2.05 g 3-[2,6-bis(benzyloxy)pyridin-3-yl]-2-methyl-5-nitro-8,8a-dihydroquinoline (4.3 mmol, 39% yield) as a pale yellow solid.

### Step 4: Synthesis of 3-(5-amino-2-methylquinolin-3-yl)piperidine-2,6-dione

Pd on activated charcoal (1.2 g) was added to a solution of 3-[2,6-bis(benzyloxy)pyridin-3-yl]-2-methyl-5-nitro-8,8a-dihydroquinoline (2.05 g, 4.29 mmol) in THF/methanol (5:1, 300 mL). The reaction mixture was stirred under H₂ atmosphere for 96 hrs. The catalyst was removed by filtration and the filtrate was evaporated under vacuum. The obtained crude product was purified by HPLC (eluent water-acetonitrile) to afford 0.05 g of the target compound 3-(5-amino-2-methylquinolin-3-yl)piperidine-2,6-dione (4% yield) as a white solid.

1H NMR: (500 MHz, DMSO-d₆) δ 10.92 (s, 1H), 8.25 (s, 1H), 7.33 (t, J=7.9 Hz, 1H) 7.07 (d, J=8.2 Hz, 1H), 6.61 (d, J=7.5 Hz, 1H), 5.86 (brs, 2H), 4.25-4.17 (m, 1H), 2.89-2.79 (m, 1H), 2.69-2.61 (m, 1H), 2.59 (s, 3H), 2.46-2.36 (m, 1H), 2.15-2.08 (m, 1H)

LCMS (m/z [M+H]⁺): 270.2

### Example 164: Synthesis of 3-(2-methyl-5-nitroquinolin-3-yl)piperidine-2,6-dione (64)

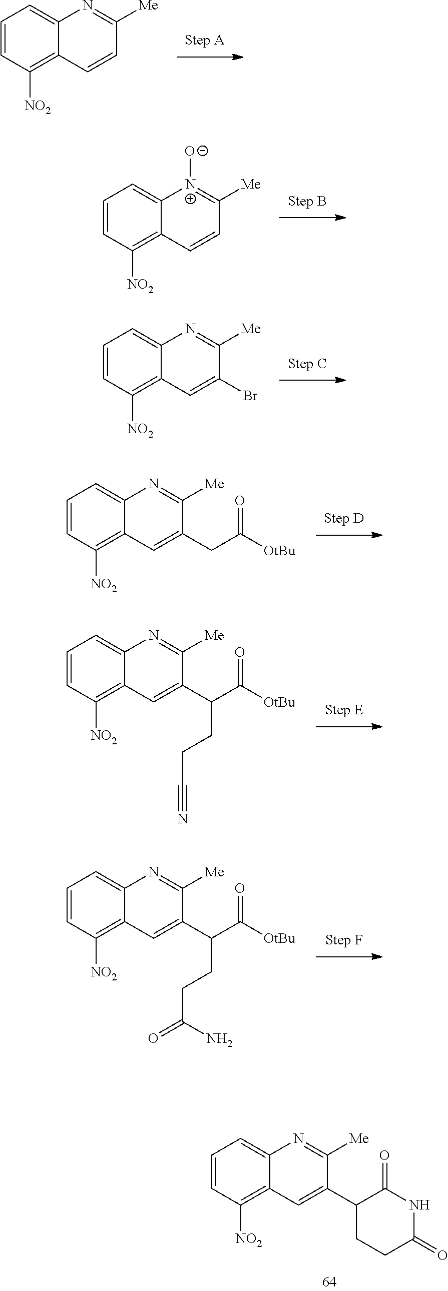

### Step A

To an ice cold solution of 5-nitro-2-methyl quinoline (2.3 g, 12.22 mmol) in DCM (25 mL) was added m-CPBA (2.3 g, 13.67 mmol). The reaction mixture was warmed to RT and stirred for 16 h. The mixture was filtered and filtrates were washed with 1 M KOH solution, dried over Na₂SO₄, and concentrated under reduced pressure to give 2-methyl-5-nitroquinoline 1-oxide (88% yield).

### Step B

To an ice cold solution of 2-methyl-5-nitroquinoline 1-oxide (500.0 mg, 2.44 mmol) in DCM (5 mL) was added POBr₃ (1.4 g, 4.9 mmol) in DCM (5 mL). The reaction mixture warmed to RT and stirred for 48 h. Ice water was added, the solution was neutralized with 10% NH₃ solution, extracted with DCM, dried over Na₂SO₄, concentrated under reduced pressure and purified by flash column chromatography to give 2-methyl-3-bromo-5-nitroquinoline (14% yield).

### Step C

To a solution of 2-methyl-3-bromo-5-nitroquinoline (600 mg, 2.24 mmol) in dioxane (8 mL) was added KOAc (441 mg, 4.49 mmol) followed by 1-(tert-butyldimethylsilyloxy)-1-tert-butoxyethylene (2.07 g, 8.98 mmol) and the reaction mixture was degassed for 15 min under N₂.

Pd[P(o-Tol)₃]2Cl₂ (353.2 mg, 0.449 mmol) was added and the reaction mixture was stirred at 130° C. for 48 h, diluted with ethyl acetate, filtered through celite bed, concentrated under reduced pressure and purified by flash column chromatography to give tert-butyl 2-(2-methyl-5-nitroquinolin-3-yl)acetate (58% yield).

### Step D

To a solution of tert-butyl 2-(2-methyl-5-nitroquinolin-3-yl)acetate (200 mg, 0.662 mmol) in DMF (10 mL) were added K₂CO₃ (150.6 mg, 0.662 mmol), benzyltriethylammonium chloride (91.4 mg, 0.662 mmol) and acrylonitrile (0.043 mL, 0.662 mmol) and the reaction mixture was stirred at RT for 16 h. The reaction mixture was diluted with water, extracted with ethyl acetate, dried over Na₂SO₄, concentrated under reduced pressure and purified by flash column chromatography to give tert-butyl 4-cyano-2-(2-methyl-5-nitroquinolin-3-yl)butanoate (40% yield).

### Step E

To an ice cold solution of tert-butyl 4-cyano-2-(2-methyl-5-nitroquinolin-3-yl)butanoate (120.0 mg, 0.338 mmol) in DMSO (5 mL) were added H₂O₂ (0.052 mL, 1.688 mmol) and K₂CO₃ (6.533 mg, 0.047 mmol). The reaction mixture warmed to RT and stirred for 16 h, diluted with water, extracted with ethyl acetate, dried over Na₂SO₄, concentrated under reduced pressure and purified by SFC to give tert-butyl 5-amino-2-(2-methyl-5-nitroquinolin-3-yl)-5-oxopentanoate (51% yield).

### Step F

In a vial were placed tert-butyl 5-amino-2-(2-methyl-5-nitroquinolin-3-yl)-5-oxopentanoate (5.0 mg, 0.013 mmol, 1.000 eq), p-toluenesulfonic acid (25.5 mg, 0.134 mmol, 10.000 eq) and acetonitrile (0.5 mL) and the reaction mixture was stirred at 80° C. for 2 h. The mixture was concentrated under reduced pressure and purified by HPLC to give 3-(2-methyl-5-nitroquinolin-3-yl)piperidine-2,6-dione (77% yield).

¹H NMR (500 MHz, DMSO) δ 10.98 (s, 1H), 8.60 (s, 1H), 8.40-8.30 (m, 2H), 7.89 (dd, J=8.5, 7.7 Hz, 1H), 4.42 (dd, J=12.5, 4.7 Hz, 1H), 2.82 (ddd, J=17.8, 12.8, 5.3 Hz, 1H), 2.71 (s, 3H), 2.66-2.61 (m, 1H), 2.44 (dd, J=12.8, 4.3 Hz, 1H), 2.14 (ddt, J=10.0, 7.8, 3.9 Hz, 1H).

LCMS (m/z [M+H]⁺): 299.9

### Example 165: Synthesis of 3-(5-fluoro-2-methylquinolin-3-yl)piperidine-2,6-dione (65)

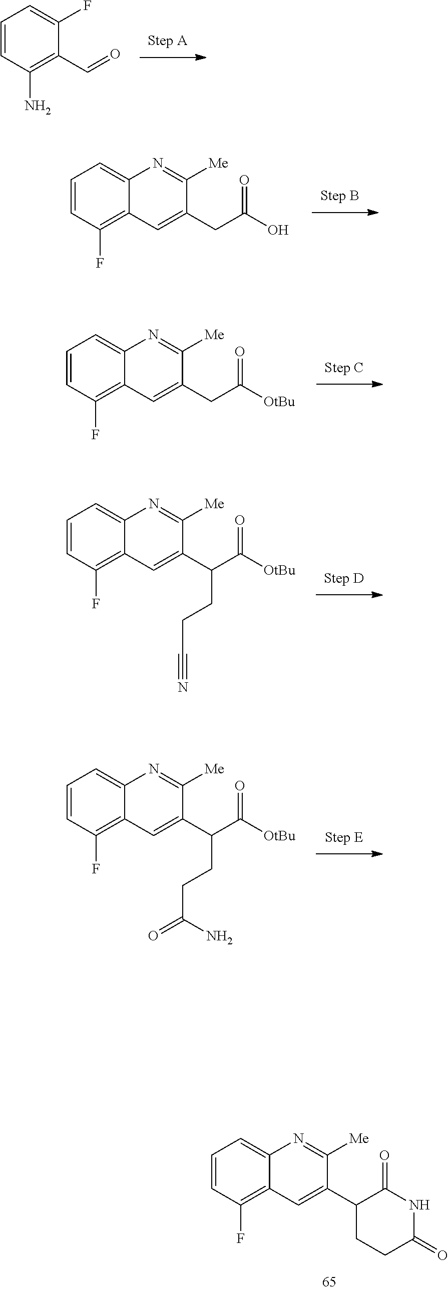 [SMILES: Nc1cccc(F)c1C=O ; Cc1nc2cccc(F)c2cc1CC(=O)O ; Cc1nc2cccc(F)c2cc1CC(=O)OC(C)(C)C ; Cc1nc2cccc(F)c2cc1C(CCC#N)C(=O)OC(C)(C)C ; Cc1nc2cccc(F)c2cc1C(CCC(N)=O)C(=O)OC(C)(C)C ; Cc1nc2cccc(F)c2cc1C1CCC(=O)NC1=O]

### Step A

To a solution of 2-amino-6-fluorobenzaldehyde (1.0 g, 7.19 mmol) in MeOH (20 mL) was added 4-oxopentanoic acid (0.739 mL, 7.194 mmol) followed by 2M NaOH (5.0 mL). The reaction mixture was refluxed for 18 h, concentrated under reduced pressure, neutralized with acetic acid, the solids were filtered and washed with ether and pentane to give 2-(5-fluoro-2-methylquinolin-3-yl)acetic acid (38%).

### Step B

To a solution of DCC (1.036 g, 5.023 mmol) in DCM (5.0 mL) were added DMAP (446 mg, 3.653 mmol) and 2-(5-fluoro-2-methylquinolin-3-yl)acetic acid (1.0 g, 4.566 mmol). Tert-butanol (0.406 mL, 13.7 mmol) was added and the reaction mixture was warmed to RT and stirred for 12 h. The reaction mixture was diluted water, extracted with ethyl acetate, dried over Na₂SO₄, concentrated under reduced pressure and purified by flash column chromatography to give tert-butyl 2-(5-fluoro-2-methylquinolin-3-yl)acetate (35% yield).

### Step C

To a solution of tert-butyl 2-(5-fluoro-2-methylquinolin-3-yl)acetate (500 mg, 1.816 mmol) in DMF (10 mL) were added K₂CO₃ (251 mg, 1.816 mmol), benzyltriethylammonium chloride (413.6 mg, 1.816 mmol) and acrylonitrile (0.119 mL, 1.816 mmol) and the reaction mixture was stirred at RT for 16 h. The reaction mixture was diluted with water, extracted with ethyl acetate, dried over Na₂SO₄, concentrated under reduced pressure and purified by flash column chromatography to give tert-butyl 4-cyano-2-(5-fluoro-2-methylquinolin-3-yl)butanoate (50% yield).

### Step D

To an ice cold solution of tert-butyl 4-cyano-2-(5-fluoro-2-methylquinolin-3-yl)butanoate (500 mg, 1.524 mmol) in DMSO (5 mL) were added H₂O₂ (0.238 mL, 7.77 mmol) and K₂CO₃ (29.5 mg, 0.14 mmol). The reaction mixture warmed to RT and stirred for 16 h, diluted with water, extracted with ethyl acetate, dried over Na₂SO₄, concentrated under reduced pressure and purified by flash column chromatography to give tert-butyl 5-amino-2-(5-fluoro-2-methylquinolin-3-yl)-5-oxopentanoate (45% yield).

### Step E

In a vial were placed tert-butyl 5-amino-2-(5-fluoro-2-methylquinolin-3-yl)-5-oxopentanoate (5.0 mg, 0.014 mmol, 1.000 eq), p-toluenesulfonic acid (27.5 mg, 0.144 mmol, 10.000 eq) and acetonitrile (0.5 mL) and the reaction mixture was stirred at 80° C. for 2 h. The mixture was concentrated under reduced pressure and purified by HPLC to give 3-(5-fluoro-2-methylquinolin-3-yl)piperidine-2,6-dione (84% yield).

¹H NMR (500 MHz, DMSO) δ 10.94 (s, 1H), 8.24 (s, 1H), 7.79 (d, J=8.5 Hz, 1H), 7.70 (td, J=8.2, 6.2 Hz, 1H), 7.37 (dd, J=10.0, 7.6 Hz, 1H), 4.36 (dd, J=12.7, 4.7 Hz, 1H), 2.82 (ddd, J=17.8, 13.2, 5.4 Hz, 1H), 2.68 (s, 3H), 2.61 (dd, J=17.4, 3.5 Hz, 1H), 2.57-2.51 (m, 1H), 2.12 (dtd, J=12.8, 5.1, 2.6 Hz, 1H).

LCMS (m/z [M+H]⁺): 272.9

### Synthesis of Ligase Ligand Moieties—Compounds of Formula (Va) and (Vb)

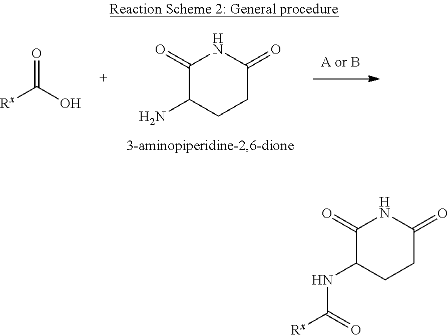

### Synthetic Conditions D

An appropriate acid (R^{x}COOH in the above reaction scheme) (1.1 eq), DMAP (0.04 eq), and EDC (1.2 eq) were added to a solution of 3-aminopiperidine-2,6-dione (1 eq) and N-hydroxybenzotriazole (1.2 eq) in DMF (0.5 M). The reaction mixture was stirred overnight at room temperature (20-25° C.).

Water (2×DMF volume) was added and the obtained solution was extracted with dichloromethane (3×DMF volume). The combined organic layers were washed with water, dried over Na₂SO₄, and concentrated under reduced pressure. The crude product was purified by preparative HPLC or by column chromatography.

### Synthetic Conditions E

An appropriate acid R^{x}COOH in the above reaction scheme) (1 eq) and EDC (1.2 eq) were added to a solution of 3-aminopiperidine-2,6-dione (hydrochloride salt, 1.1 eq), triethylamine (1.2 eq) and N-hydroxybenzotriazole (1.2 eq) in DMA (0.5 M). The reaction mixture was stirred overnight at rt. Water (2×DMA volume) was added and obtained mixture was extracted with dichloromethane (3×DMA volume). The combined organic layers were washed with water, dried over Na₂SO₄, and concentrated under reduced pressure. The crude product was isolated by preparative HPLC or by column chromatography.

### Synthetic Conditions F

To a solution of appropriate acid (R^{x}COOH in the above reaction scheme) (1 eq) and HATU (1.5 eq) in dry DMF were added 3-aminopiperidine-2,6-dione (hydrochloride salt, 1.2 eq) and DIPEA (3 eq). The reaction mixture was stirred overnight at rt. The crude product was purified by preparative HPLC or/and by preparative TLC.

### Synthetic Conditions G

To a solution of appropriate acid (R^{x}COOH in the above reaction scheme) (1 eq) 3-aminopiperidine-2,6-dione (hydrochloride salt, 1.2 eq) and DMAP (0.1 eq.) in an inert atmosphere in dry DMF were added DIPEA (2.2 eq.) and HATU (1.5 eq) in dry DMF. The reaction mixture was stirred overnight at rt. The crude product was purified by preparative HPLC or/and by preparative TLC.

### Example Method 1: Formation of Chlorinated R^{x} Group of R^{x}COOH (or its Ester R^{x}COOR′)

N-chlorosuccinimide (1.1 eq) was added to a solution of an appropriate starting material (1 eq) in DMF (0.5 M) and the reaction mixture was stirred for 2 h at room temperature (20-25° C.). The reaction mixture was poured into water (2×DMF volume) and occurred precipitate was filtered. The solids were washed with water and dried in vacuum to give the acid, R^{x}COOH.

### Example Method 2: Synthesis of R^{x}COOH from Corresponding Ester R^{x}COOR′)

LiOH (1.1 eq) was added to a solution of an appropriate ester (1 eq) in THF:water mixture (3:1 or 5:1, 85 mM) and the resulting mixture was stirred overnight at room temperature (20-25° C.). The mixture was concentrated under reduced pressure, diluted with water, and acidified with concentrated HCl to pH=2-3. The precipitate was filtered, washed with water, and dried in vacuum to give the target carboxylic acid.

### Example Method 3: Formation of Acetylated R^{x} Group of R^{x}COOR′

A mixture of an appropriate amine (1 eq.), Ac₂O (3 eq.), and DMAP (0.2 eq.) in dioxane (0.2 M) was heated to 80° C. for 2 h. Upon completion, the mixture was cooled down to room temperature (20-25° C.) and concentrated under reduced pressure. The residue was diluted with water (1× dioxane volume) and extracted with EtOAc (3× dioxane volume). The organic layers were washed with water, brine, dried over Na₂SO₄, and evaporated to dryness to afford an acylated product typically used without further purification.

TABLE 7

| Compound | | |
|---|---|---|
| number | Structure | Synthesis method |
| 67 | 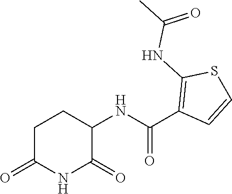 [SMILES: CC(=O)Nc1sccc1C(=O)NC1CCC(=O)NC1=O] | Synthetic Conditions E (2-acetamidothiophene-3-carboxylic acid starting material) |
| 69 | 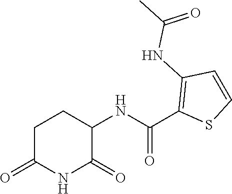 [SMILES: CC(=O)Nc1ccsc1C(=O)NC1CCC(=O)NC1=O] | Synthetic Conditions E (N-(2,6-dioxopiperidin-3-yl)-3- acetamidothiophene-2-carboxamide starting material) |
| 72 | 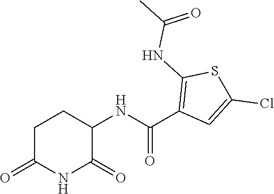 [SMILES: CC(=O)Nc1sc(Cl)cc1C(=O)NC1CCC(=O)NC1=O] | Synthetic Conditions E (5-chloro-2-acetamidothiophene-3- carboxylic acid starting material) |
| 73 | 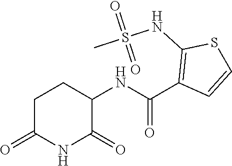 [SMILES: CS(=O)(=O)Nc1sccc1C(=O)NC1CCC(=O)NC1=O] | Synthetic Conditions E (2-(methylsulfonamido)thiophene-3- carboxylic acid starting material) |
| 75 | 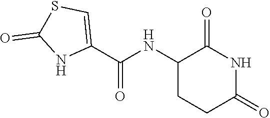 [SMILES: O=C1CCC(NC(=O)c2csc(=O)[nH]2)C(=O)N1] | Synthetic Conditions E (2-oxo-2,3-dihydrothiazole-4-carboxylic acid starting material) |
| 78 | 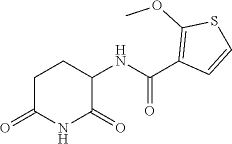 [SMILES: COc1sccc1C(=O)NC1CCC(=O)NC1=O] | Synthetic Conditions E (2-methoxythiophene-3-carboxylic acid starting material) |
| 79 | 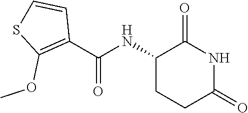 [SMILES: COc1sccc1C(=O)N[C@H]1CCC(=O)NC1=O] | Synthetic Conditions G (2-methoxythiophene-3-carboxylic acid (20 mg) and (S)-3-aminopiperidine-2,6- dione starting material) |
| 80 | 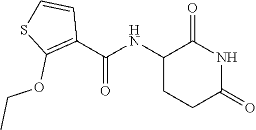 [SMILES: CCOc1sccc1C(=O)NC1CCC(=O)NC1=O] | Synthetic Conditions G (2-ethoxythiophene-3-carboxylic acid starting material) |
| 81 | 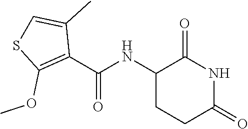 [SMILES: COc1scc(C)c1C(=O)NC1CCC(=O)NC1=O] | Synthetic Conditions F (2-methoxy-4-methylthiophene-3- carboxylic acid starting material) |
| 82 | 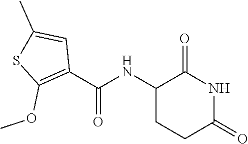 [SMILES: COc1sc(C)cc1C(=O)NC1CCC(=O)NC1=O] | Synthetic Conditions F (2-methoxy-5-methylthiophene-3- carboxylic acid a starting material) |
| 85 | 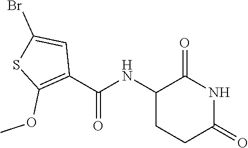 [SMILES: COc1sc(Br)cc1C(=O)NC1CCC(=O)NC1=O] | Synthetic Conditions G (5-bromo-2-methoxythiophene-3- carboxylic acid starting material) |
| 88 | 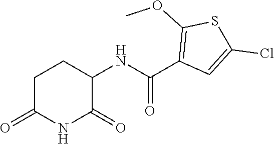 [SMILES: COc1sc(Cl)cc1C(=O)NC1CCC(=O)NC1=O] | Synthetic Conditions D (5-chloro-2-methoxythiophene-3- carboxylic acid starting material) |
| 93 | 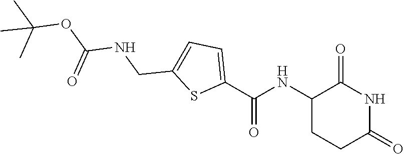 [SMILES: CC(C)(C)OC(=O)NCc1ccc(C(=O)NC2CCC(=O)NC2=O)s1] | Synthetic Conditions F (5-[(tert- butoxycarbonylamino)methyl]thiophene- 2-carboxylic acid starting material |
| 94 | 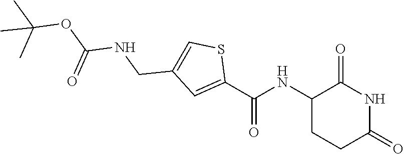 [SMILES: CC(C)(C)OC(=O)NCc1csc(C(=O)NC2CCC(=O)NC2=O)c1] | Synthetic Conditions F (4-((tert- butoxycarbonyl)aminomethyl)thiophene- 2-carboxylic acid starting material |
| 95 | 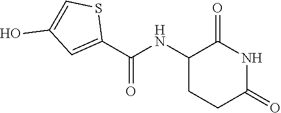 [SMILES: O=C1CCC(NC(=O)c2cc(O)cs2)C(=O)N1] | Synthetic Conditions G (4-hydroxythiophene-2-carboxylic acid starting material |
| 96 | 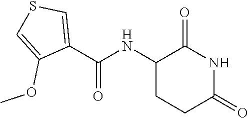 [SMILES: COc1cscc1C(=O)NC1CCC(=O)NC1=O] | Synthetic Conditions G (4-methoxythiophene-3-carboxylic acid starting material) |
| 97 | 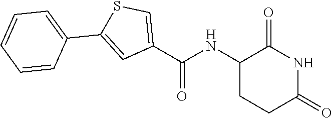 [SMILES: O=C1CCC(NC(=O)c2csc(-c3ccccc3)c2)C(=O)N1] | Synthetic Conditions G (5-phenylthiophene-3-carboxylic acid starting material) |
| 98 | 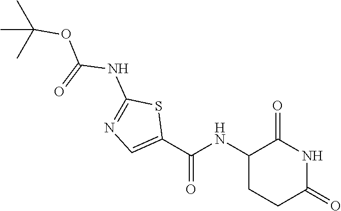 [SMILES: CC(C)(C)OC(=O)Nc1ncc(C(=O)NC2CCC(=O)NC2=O)s1] | Synthetic Conditions F (2-((tert- butoxycarbonyl)amino)thiazole-5- carboxylic acid starting material |
| 99 | 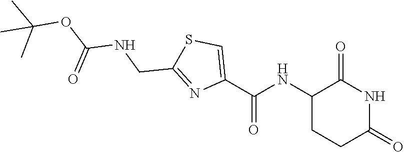 [SMILES: CC(C)(C)OC(=O)NCc1nc(C(=O)NC2CCC(=O)NC2=O)cs1] | Synthetic Conditions F (2-(((tert- butoxycarbonyl)amino)methyl)thiazole- 4-carboxylic acid starting material |
| 104 | 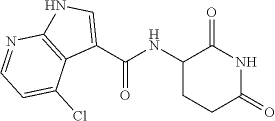 [SMILES: O=C1CCC(NC(=O)c2c[nH]c3nccc(Cl)c23)C(=O)N1] | Synthetic Conditions F (4-chloro-1H-pyrrolo[2,3-b]pyridine-3- carboxylic acid starting material |
| 105 | 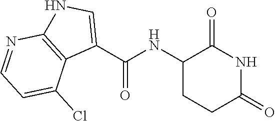 [SMILES: O=C1CCC(NC(=O)c2c[nH]c3nccc(Cl)c23)C(=O)N1] | Synthetic Conditions F (5-chloro-1H-pyrrolo[2,3-b]pyridine-3- carboxlic acid starting material. |

### Example 166: Synthesis of N-(2,6-dioxopiperidin-3-yl)-2-(N-methylacetamido)thiophene-3-carboxamide (66)

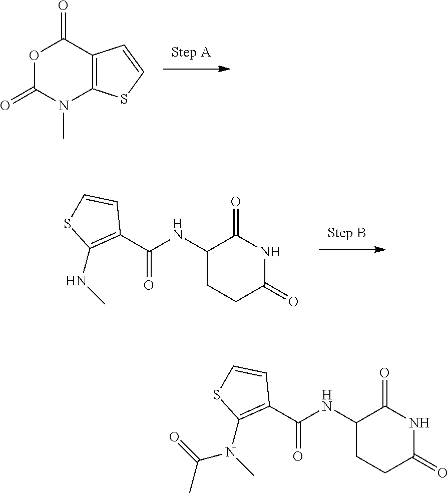 [SMILES: Cn1c(=O)oc(=O)c2ccsc21 ; CNc1sccc1C(=O)NC1CCC(=O)NC1=O ; CC(=O)N(C)c1sccc1C(=O)NC1CCC(=O)NC1=O]

### Step A

3-Aminopiperidine-2,6-dione (3.3 g, 25.8 mmol) and triethylamine (2.45 g, 24.2 mmol) were added to a solution of 1-methyl-1H,2H,4H-thieno[2,3-d][1,3]oxazine-2,4-dione (3.7 g, 20.2 mmol) in ethanol (20 mL). The reaction mixture was refluxed for 16 h and filtered. The precipitate was washed with water to give N-(2,6-dioxopiperidin-3-yl)-2-(methylamino)thiophene-3-carboxamide (19% yield).

### Step B

Acetic anhydride (0.265 g, 2.60 mmol) and DMAP (0.026 g, 0.213 mmol) were added to a solution of N-(2,6-dioxopiperidin-3-yl)-2-(methylamino)thiophene-3-carboxamide (0.579 g, 2.17 mmol) and triethylamine (0.263 g, 2.60 mmol) in dioxane (10 mL). The reaction mixture was stirred at 60° C. for 16 h, washed with water and extracted with EtOAc (3×10 mL), dried over Na₂SO₄, concentrated under reduced pressure and purified by HPLC to give N-(2,6-dioxopiperidin-3-yl)-2-(N-methylacetamido)thiophene-3-carboxamide (11% yield).

¹H NMR (500 MHz, DMSO) δ 10.86 (s, 1H), 8.53 (d, J=8.0 Hz, 1H), 7.56 (d, J=5.7 Hz, 1H), 7.34 (d, J=5.7 Hz, 1H), 4.72-4.62 (m, 1H), 3.09 (s, 3H), 2.83-2.71 (m, 1H), 2.58-2.53 (m, 1H), 2.19-2.02 (m, 1H), 1.99-1.90 (m, 1H), 1.81 (s, 3H).

LCMS (m/z [M+H]⁺): 310.2

### Example 167: Synthesis of 5-chloro-2-cyclopropaneamido-N-(2,6-dioxopiperidin-3-yl)thiophene-3-carboxamide (68)

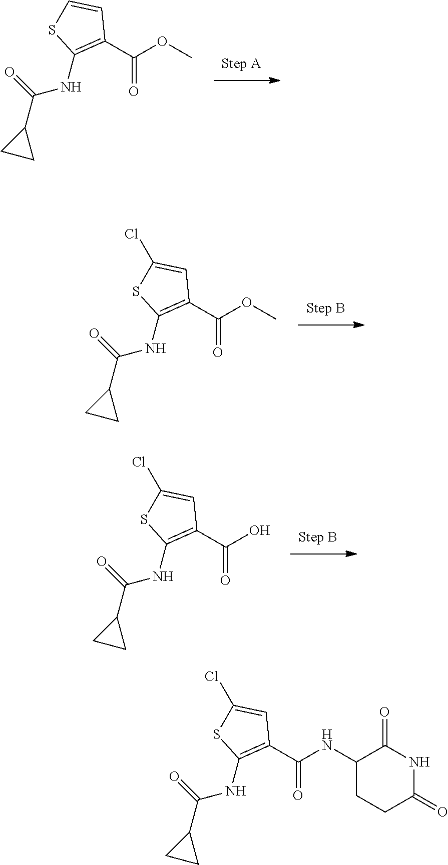 [SMILES: COC(=O)c1ccsc1NC(=O)C1CC1 ; COC(=O)c1cc(Cl)sc1NC(=O)C1CC1 ; O=C(O)c1cc(Cl)sc1NC(=O)C1CC1 ; O=C1CCC(NC(=O)c2cc(Cl)sc2NC(=O)C2CC2)C(=O)N1]

### Step A

Methyl 5-chloro-2-cyclopropaneamidothiophene-3-carboxylate was synthesized using Example Method 1, above (80% yield) using methyl 2-cyclopropaneamidothiophene-3-carboxylate as a starting material.

### Step B

5-chloro-2-cyclopropaneamidothiophene-3-carboxylic acid was synthesized using Example Method 2, above (86% yield) using methyl 5-chloro-2-cyclopropaneamidothiophene-3-carboxylate as a starting material.

### Step C

5-chloro-2-cyclopropaneamido-N-(2,6-dioxopiperidin-3-yl)thiophene-3-carboxamide was synthesized using the general procedure shown in Reaction Scheme 1 and Synthetic Conditions E, above (30% yield), using 5-chloro-2-cyclopropaneamidothiophene-3-carboxylic acid as a starting material.

¹H NMR (500 MHz, DMSO) δ 12.11 (s, 1H), 10.88 (s, 1H), 8.69-8.60 (m, 1H), 7.50 (s, 1H), 4.79-4.69 (m, 1H), 2.84-2.72 (m, 1H), 2.61-2.53 (m, 1H), 2.21-2.08 (m, 1H), 2.04-1.92 (m, 2H), 0.98-0.84 (m, 4H).

LCMS (m/z [M+H]⁺): 356.2

### Example 168: Synthesis of N-(2,6-dioxopiperidin-3-yl)-2-acetamido-4-methoxythiophene-3-carboxamide (70)

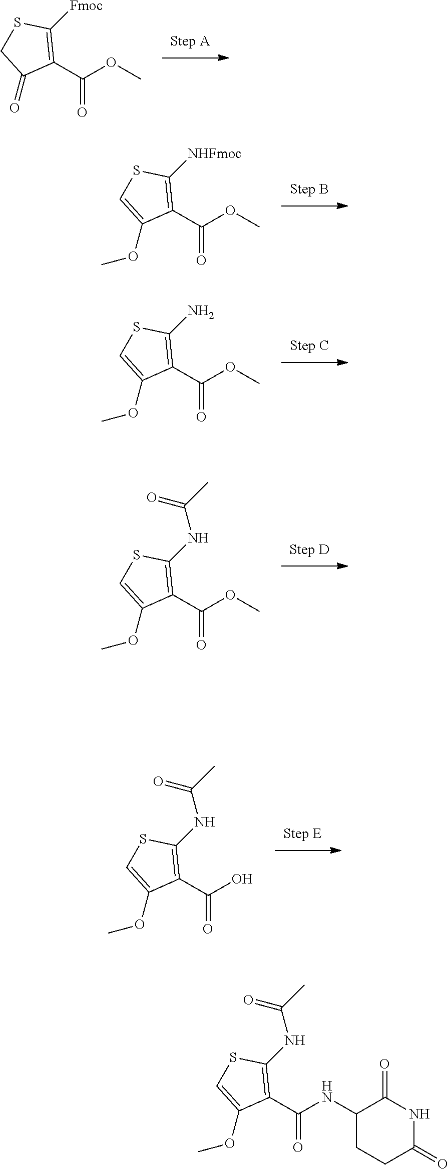

### Step A

H₂SO₄ (1 mL) was added dropwise to a stirred suspension of methyl 2-({[(9H-fluoren-9-yl)methoxy]carbonyl}amino)-4-oxo-4,5-dihydrothiophene-3-carboxylate (9.65 g, 24.4 mmol) in MeOH (200 mL). The reaction mixture was refluxed for 16 h, cooled to RT and filtered to give 2-({[(9H-fluoren-9-yl)methoxy]carbonyl}amino)-4-methoxythiophene-3-carboxylate (63% yield).

### Step B

Morpholine (13.5 g, 155 mmol) was added to a solution of methyl 2-({[(9H-fluoren-9-yl)methoxy]carbonyl}amino)-4-methoxythiophene-3-carboxylate (6.3 g, 15.4 mmol) in dichloromethane (100 mL) and the reaction mixture was stirred overnight at room temperature, concentrated under reduced pressure, diluted with MTBE, filtered, and rinsed with small amount of MTBE. The filtrate was evaporated in vacuo to give crude methyl 2-amino-4-methoxythiophene-3-carboxylate, which was used in the next step without further purification.

### Step C

methyl 2-acetamido-4-methoxythiophene-3-carboxylate was obtained in 73% yield using Example Method 3, above, with methyl 2-amino-4-methoxythiophene-3-carboxylate as a starting material.

### Step D

2-acetamido-4-methoxythiophene-3-carboxylic acid was obtained in 20% yield using Example Method 2, above, with methyl 2-acetamido-4-methoxythiophene-3-carboxylate as a starting material.

### Step E

N-(2,6-dioxopiperidin-3-yl)-2-acetamido-4-methoxythiophene-3-carboxamide was synthesized using the general procedure shown in Reaction Scheme 1 and Synthetic Conditions E, above (47% yield), and 2-acetamido-4-methoxythiophene-3-carboxylic acid as a starting material.

¹H NMR (500 MHz, DMSO) δ 12.05 (s, 1H), 10.92 (s, 1H), 8.30 (d, J=7.1 Hz, 1H), 6.14 (s, 1H), 4.76-4.66 (m, 1H), 3.83 (s, 3H), 2.82-2.70 (m, 1H), 2.58-2.52 (m, 1H), 2.19 (s, 3H), 2.16-2.06 (m, 2H)

LCMS (m/z [M+H]⁺): 326.2

### Example 169: Synthesis of 5-cyano-N-(2,6-dioxopiperidin-3-yl)-2-acetamidothiophene-3-carboxamide (71)

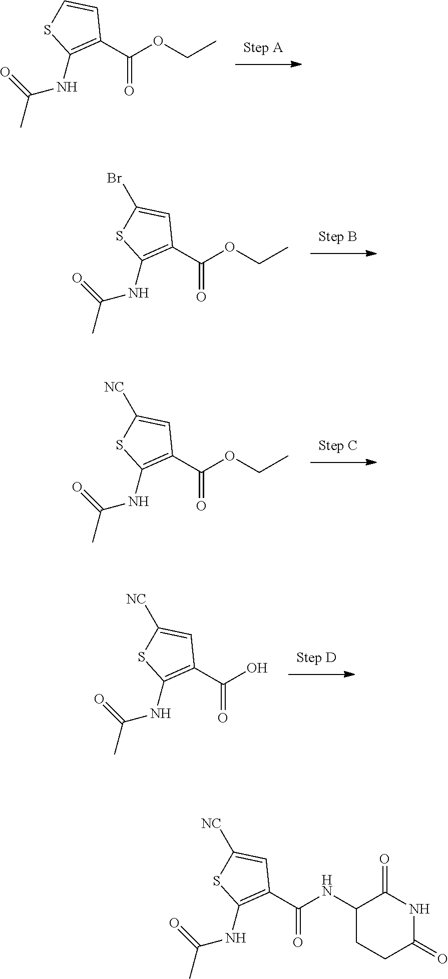 [SMILES: CCOC(=O)c1ccsc1NC(C)=O ; CCOC(=O)c1cc(Br)sc1NC(C)=O ; CCOC(=O)c1cc(C#N)sc1NC(C)=O ; CC(=O)Nc1sc(C#N)cc1C(=O)O ; CC(=O)Nc1sc(C#N)cc1C(=O)NC1CCC(=O)NC1=O]

### Step A

Ethyl 2-acetamidothiophene-3-carboxylate (11 g, 51.6 mmol) was dissolved in AcOH (110 mL) and solution of bromine (3.2 mL, 61.9 mmol) in AcOH (55 mL) was added dropwise over 15 min at RT. The reaction mixture was stirred at RT for 18 h, concentrated under reduced pressure and diluted water. The precipitate was filtered, washed with water and dried to give ethyl 5-bromo-2-acetamidothiophene-3-carboxylate (93% yield).

### Step B

Zn(CN)₂ (8.45 g, 72 mmol) and Pd(dppf)Cl₂-DCM (3.92 g, 4.8 mmol) were added to a solution of ethyl 5-bromo-2-acetamidothiophene-3-carboxylate (14 g, 48 mmol) in DMF (120 mL). Argon was bubbled through the reaction mixture for 10 min, then the reaction mixture was stirred at 150° C. for 16 h, cooled to RT, filtered and washed with EtOAc. The organic layer was dried over Na₂SO₄, concentrated under reduced pressure and purified by flash column chromatography to give 5-cyano-2-acetamidothiophene-3-carboxylate (83% yield).

### Step C

Ethyl 5-cyano-2-acetamidothiophene-3-carboxylate (9.45 g, 39.7 mmol) was dissolved in EtOH:THF solution (120 mL:360 mL), the solution was cooled to +5° C. and lithium hydroxide monohydrate (11.7 g, 278 mmol) in H₂O (120 mL) was added dropwise over 20 min. The reaction mixture was stirred at RT for 18 h, concentrated under reduced pressure and acidified with 15% citric acid. The product was extracted with EtOAc, dried over Na₂SO₄ and evaporated under reduced pressure to give 5-cyano-2-acetamidothiophene-3-carboxylic acid (57% yield).

### Step D

5-cyano-N-(2,6-dioxopiperidin-3-yl)-2-acetamidothiophene-3-carboxamide was synthesized using the general procedure shown in Reaction Scheme 1 and Synthetic Conditions D, above (35% yield), and 5-cyano-2-acetamidothiophene-3-carboxylic acid as a starting material.

¹H NMR (400 MHz, DMSO) δ 12.08 (s, 1H), 10.94 (s, 1H), 8.89 (d, J=7.9 Hz, 1H), 8.27 (s, 1H), 4.82-4.65 (m, 1H), 2.87-2.72 (m, 1H), 2.62-2.53 (m, 1H), 2.30 (s, 3H), 2.24-2.08 (m, 1H), 2.06-1.93 (m, 1H)

LCMS (m/z [M+H]⁺): 321.0

### Example 170: Synthesis of 5-acetamido-N⁴-(2,6-dioxopiperidin-3-yl)-5-acetamido-N²-methylthiophene-2,4-dicarboxamide (74)

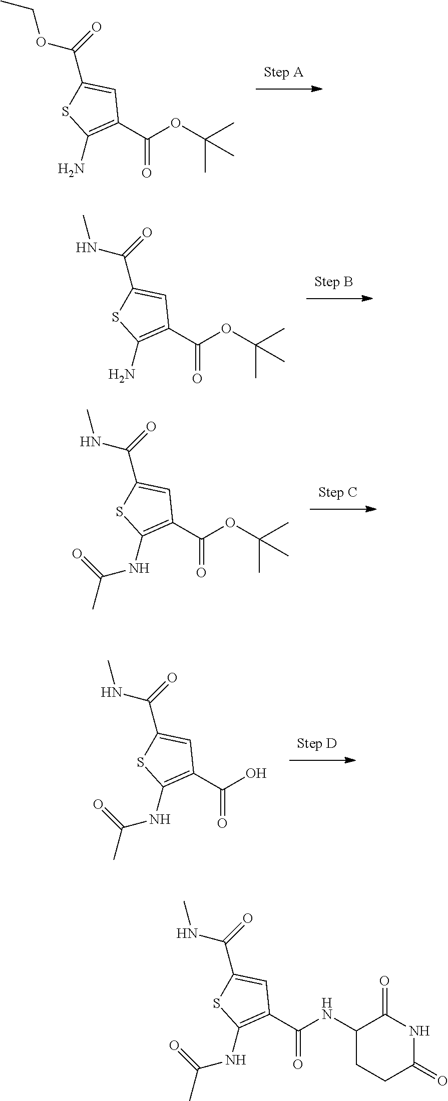 [SMILES: CCOC(=O)c1cc(C(=O)OC(C)(C)C)c(N)s1 ; CNC(=O)c1cc(C(=O)OC(C)(C)C)c(N)s1 ; CNC(=O)c1cc(C(=O)OC(C)(C)C)c(NC(C)=O)s1 ; CNC(=O)c1cc(C(=O)O)c(NC(C)=O)s1 ; CNC(=O)c1cc(C(=O)NC2CCC(=O)NC2=O)c(NC(C)=O)s1]

### Step A

4-tert-butyl 2-ethyl 5-aminothiophene-2,4-dicarboxylate (3.71 g, 13.7 mmol) was added to 20% solution of methylamine in methanol (20 mL) and the reaction mixture was stirred for 5 days at 70° C., concentrated under reduced pressure and triturated with isopropyl alcohol:hexane (1:1). The precipitate was filtered to give tert-butyl 2-amino-5-(methylcarbamoyl)thiophene-3-carboxylate (93% yield).

### Step B

Triethylamine (3.3 g, 32.6 mmol), DMAP (0.13 g, 1.06 mmol) and acetic acid (1.67 g, 27.8 mmol) were added to a solution of tert-butyl 2-amino-5-(methylcarbamoyl)thiophene-3-carboxylate (2.8 g, 10.9 mmol) in dry MeCN (30 mL). The reaction mixture was stirred overnight at 50° C., cooled to room temperature, diluted with water, extracted with DCM, dried over Na₂SO₄ and concentrated under reduced pressure to give tert-butyl 2-acetamido-5-(methylcarbamoyl)thiophene-3-carboxylate (95% yield).

### Step C

10% HCl in dioxane (20 mL) was added to a solution of tert-butyl 2-acetamido-5-(methylcarbamoyl)thiophene-3-carboxylate (3.1 g, 10.4 mmol) in DCM (20 mL) and the reaction mixture was stirred for 3 days at RT. The precipitate was filtered, washed with DCM and dried to give 2-acetamido-5-(methylcarbamoyl)thiophene-3-carboxylic acid (60% yield).

### Step D

N⁴-(2,6-dioxopiperidin-3-yl)-5-acetamido-N²-methylthiophene-2,4-dicarboxamide was synthesized using the general procedure shown in Reaction Scheme 1 and Synthetic Conditions E, above (44% yield), and 2-acetamido-5-(methylcarbamoyl)thiophene-3-carboxylic acid as a starting material.

¹H NMR (400 MHz, DMSO) δ 11.84 (s, 1H), 10.92 (s, 1H), 8.78 (d, J=8.1 Hz, 1H), 8.35-8.25 (m, 1H), 7.96 (s, 1H), 4.81-4.68 (m, 1H), 2.85-2.74 (m, 1H), 2.73 (d, J=4.4 Hz, 3H), 2.62-2.52 (m, 1H), 2.24 (s, 3H), 2.20-2.07 (m, 1H), 2.04-1.93 (m, 1H)

LCMS (m/z [M+H]⁺): 352.9

### Example 171: Synthesis of 5-chloro-N-(2,6-dioxopiperidin-3-yl)-2-(methylamino)thiophene-3-carboxamide (76)

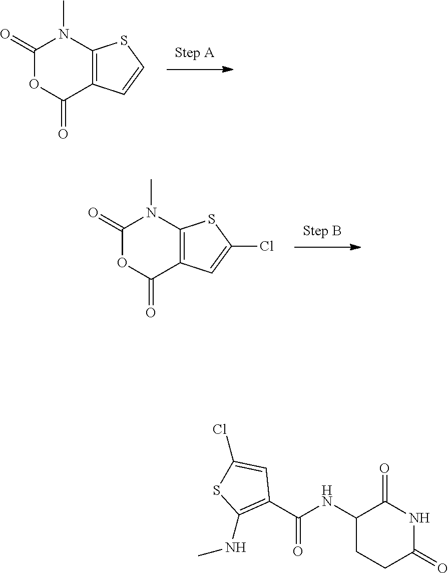 [SMILES: Cn1c(=O)oc(=O)c2ccsc21 ; Cn1c(=O)oc(=O)c2cc(Cl)sc21 ; CNc1sc(Cl)cc1C(=O)NC1CCC(=O)NC1=O]

### Step A

N-chlorosuccinimide (0.884 g, 6.62 mmol) was added to a solution of 1-methyl-1H,2H,4H-thieno[2,3-d][1,3]oxazine-2,4-dione (1 g, 5.46 mmol) in mixture of toluene (4 mL) and acetic acid (4 mL). The reaction mixture was stirred at 70° C. for 2 h, concentrated under reduced pressure, diluted with water and filtered. The solids were washed with water and dried 6-chloro-1-methyl-1H,2H,4H-thieno[2,3-d][1,3]oxazine-2,4-dione (72% yield).

### Step B

3-Aminopiperidine-2,6-dione hydrochloride (0.655 g, 3.98 mmol) and triethylamine (0.483 g, 4.77 mmol) were added to a solution of 6-chloro-1-methyl-1H,2H,4H-thieno[2,3-d][1,3]oxazine-2,4-dione (0.865 g, 3.97 mmol) in ethanol (20 mL) and the reaction mixture was refluxed for 18 h, concentrated under reduced pressure and diluted with water. The precipitate was filtered, washed with water and isopropyl alcohol, and dried to give 5-chloro-N-(2,6-dioxopiperidin-3-yl)-2-(methylamino)thiophene-3-carboxamide (44% yield).

¹H NMR (400 MHz, DMSO) δ 10.79 (s, 1H), 8.18-8.04 (m, 1H), 8.03-7.91 (m, 1H), 7.26 (s, 1H), 4.68-4.52 (m, 1H), 2.85 (s, 3H), 2.79-2.67 (m, 1H), 2.60-2.53 (m, 1H), 2.16-2.01 (m, 1H), 1.99-1.85 (m, 1H).

LCMS (m/z [M+H]⁺): 302.2

### Example 172: Synthesis of 4-chloro-5-cyclopropyl-N-(2,6-dioxopiperidin-3-yl)-2-acetamidothiophene-3-carboxamide (77)

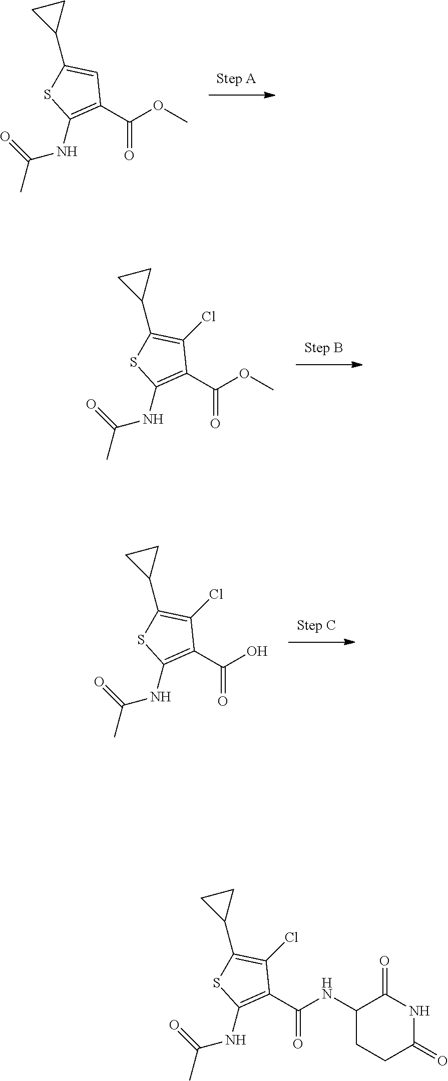 [SMILES: COC(=O)c1cc(C2CC2)sc1NC(C)=O ; COC(=O)c1c(NC(C)=O)sc(C2CC2)c1Cl ; CC(=O)Nc1sc(C2CC2)c(Cl)c1C(=O)O ; CC(=O)Nc1sc(C2CC2)c(Cl)c1C(=O)NC1CCC(=O)NC1=O]

### Step A

SO₂Cl₂ (0.207 g, 1.53 mmol) was added to a solution of methyl 5-cyclopropyl-2-acetamidothiophene-3-carboxylate (0.306 g, 1.28 mmol) in CHCl₃ (15 mL). The reaction mixture was refluxed for 2 h, concentrated under reduced pressure and diluted with water. The product was extracted with EtOAc, dried over Na₂SO₄ and concentrated under reduced pressure to give methyl 4-chloro-5-cyclopropyl-2-acetamidothiophene-3-carboxylate (81% yield).

### Step B

4-chloro-5-cyclopropyl-2-acetamidothiophene-3-carboxylic acid was obtained in 78% yield using Example Method 2, above, with methyl 4-chloro-5-cyclopropyl-2-acetamidothiophene-3-carboxylate as a starting material.

### Step C

HATU (0.370 g, 0.973 mmol) was added to the solution of 4-chloro-5-cyclopropyl-2-acetamidothiophene-3-carboxylic acid (0.211 g, 0.812 mmol), 3-aminopiperidine-2,6-dione (0.134 g, 1.05 mmol) and N-methylmorpholine (0.205 g, 2.03 mmol) in DMF (5 mL) at 0° C. The reaction mixture was stirred overnight at room temperature, diluted with water, extracted with AcOEt, dried over Na₂SO₄, concentrated under reduced pressure and purified by HPLC to give 4-chloro-5-cyclopropyl-N-(2,6-dioxopiperidin-3-yl)-2-acetamidothiophene-3-carboxamide (41% yield).

¹H NMR (400 MHz, DMSO) δ 11.07 (s, 1H), 11.04 (s, 1H), 8.54 (d, J=8.2 Hz, 1H), 4.90-4.78 (m, 1H), 2.89-2.72 (m, 1H), 2.65-2.52 (m, 2H), 2.16 (s, 3H), 2.12-1.98 (m, 2H), 1.08-0.98 (m, 2H), 0.71-0.58 (m, 2H).

LCMS (m/z [M+H]⁺): 369.8

### Example 173: Synthesis of 5-cyclopropyl-N-(2,6-dioxopiperidin-3-yl)-2-methoxythiophene-3-carboxamide (83)

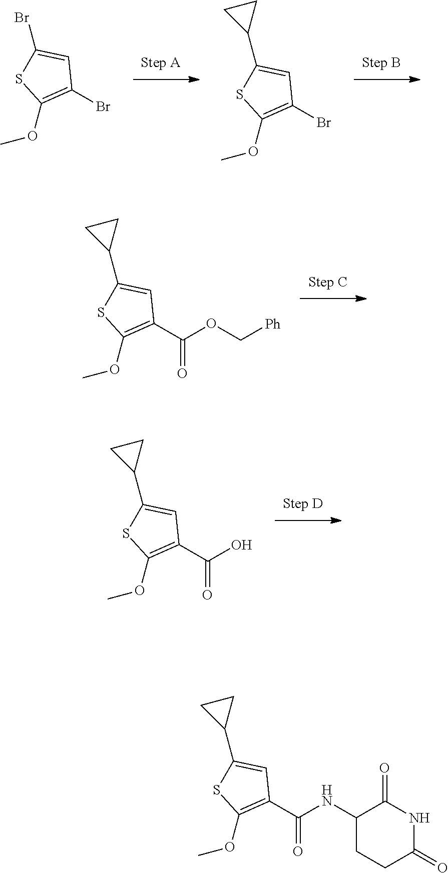

### Step A

To a stirred solution of 3,5-dibromo-2-methoxythiophene (500.0 mg, 1.845 mmol) in toluene (9 mL) was added cyclopropyl boronic acid (206 mg, 2.399 mmol) and K₃PO₄ (784 mg, 3.69 mmol) in water (3 ml), the reaction mixture was purged with argon for 15 min and then Pd(PPh₃)₄ (320 mg, 0.277 mmol) was added. The reaction was stirred at 90° C. for 20 h, filtered through celite bed, concentrated under reduced pressure and purified by flash column chromatography to give 3-bromo-5-cyclopropyl-2-methoxythiophene (34% yield).

### Step B

To a stirred solution of 3-bromo-5-cyclopropyl-2-methoxythiophene (700 mg, 3 mmol) in THE (20 mL) was added n-BuLi (1.8 M in THF) (3.4 mL, 6.005 mmol) dropwise at −78° C. Reaction mixture was stirred for 1 h at −78° C. and benzyl chloroformate (0.86 mL, 6 mmol) was added dropwise. The reaction was continued for 1 h, quenched with water, extracted with ethyl acetate and concentrated under reduced pressure. The product was purified by flash column chromatography to give benzyl 5-cyclopropyl-2-methoxythiophene-3-carboxylate (23% yield).

### Step C

To a stirred solution of benzyl 5-cyclopropyl-2-methoxythiophene-3-carboxylate (350 mg, 1.215 mmol) in THE (6 mL) and methanol (6 mL) at 5-10° C. was added 50% aq. NaOH (12 ml). The reaction mixture was stirred at RT for 16 h and acidified with 6 M HCl. The solids were filtered, washed with pentane and dried to give 5-cyclopropyl-2-methoxythiophene-3-carboxylic acid (76% yield).

### Step D

5-cyclopropyl-N-(2,6-dioxopiperidin-3-yl)-2-methoxythiophene-3-carboxamide was synthesized using the general procedure shown in Reaction Scheme 1 and Synthetic Conditions C, above, (76% yield) using 5-cyclopropyl-2-methoxythiophene-3-carboxylic acid (20 mg) as a starting material.

¹H NMR (500 MHz, DMSO) δ 10.85 (s, 1H), 7.74 (d, J=7.5 Hz, 1H), 6.78 (d, J=1.0 Hz, 1H), 4.67 (ddd, J=12.1, 7.5, 5.8 Hz, 1H), 4.00 (s, 3H), 2.77 (ddd, J=17.3, 13.1, 6.1 Hz, 1H), 2.60-2.51 (m, 1H), 2.15-1.95 (m, 3H), 0.95-0.88 (m, 2H), 0.65-0.59 (m, 2H).

LCMS (m/z [M+H]⁺): 309.0

### Example 174: Synthesis of N-(2,6-dioxopiperidin-3-yl)-2-methoxy-5-phenylthiophene-3-carboxamide (84)

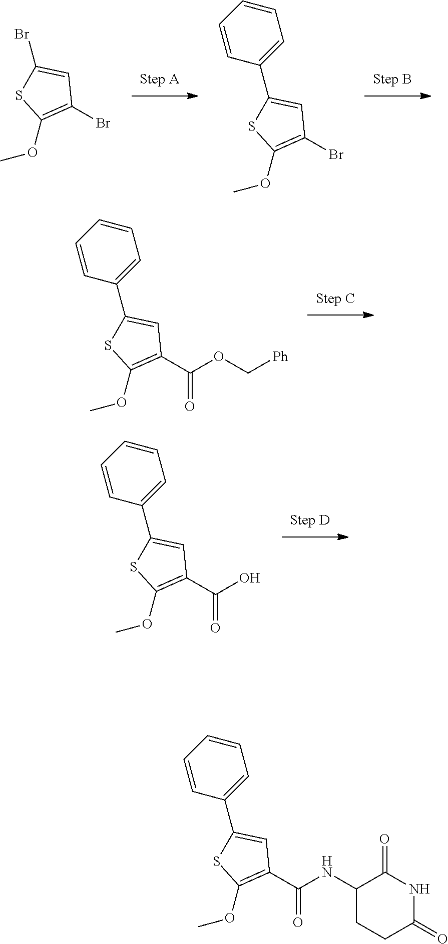

### Step A

To 3,5-dibromo-2-methoxythiophene (4.0 g, 14.71 mmol) in dry THE (30 mL) was added 2.5M n-BuLi hexane solution (6.47 mL, 16.2 mmol) at −78° C. under argon atmosphere and the solution was stirred for 1 h. Tri-n-butyl borate (8.35 mL, 29.42 mmol) was added to the reaction mixture, the mixture was stirred for 1.5 h and warmed to RT. 20% Na₂CO₃ (33.6 mL), iodobenzene (1.65 mL, 14.71 mmol), and Pd(PPh₃)₄ (0.85 g, 0.73 mmol) were added and the reaction mixture was refluxed for 16 h. The reaction mixture was extracted with ether, dried over MgSO₄, concentrated under reduced pressure and purified by flash column chromatography to give 3-bromo-2-methoxy-5-phenylthiophene (50% yield).

### Step B

3-Bromo-2-methoxy-5-phenylthiophene (900 mg, 3.34 mmol) was dissolved in THE (15 mL) and cooled to −78° C. 1.8M n-BuLi in hexane (3.7 mL, 6.68 mmol) was added dropwise at −78° C.

Reaction mixture was stirred for 1 h at −78° C. and benzyl chloroformate (0.95 mL, 6.68 mmol) was added dropwise. The reaction was continued for 1 h, quenched with water, extracted with ethyl acetate and concentrated under reduced pressure. The product was purified by flash column chromatography to give benzyl 2-methoxy-5-phenylthiophene-3-carboxylate (23% yield).

### Step C

Benzyl 2-methoxy-5-phenylthiophene-3-carboxylate (230 mg, 0.71 mmol) was dissolved in THE (5 mL). MeOH (5 mL) and 50% NaOH solution (10 mL) were added and the reaction mixture was stirred at RT for 16 h and acidified with 6 M HCl. The solids were filtered, washed with pentane and dried to give 2-methoxy-5-phenylthiophene-3-carboxylic acid (130 mg, 78%) as off white solid.

### Step D

N-(2,6-dioxopiperidin-3-yl)-2-methoxy-5-phenylthiophene-3-carboxamide was synthesized using the general procedure shown in Reaction Scheme 1 and Synthetic Conditions C, above, (71% yield) using 2-methoxy-5-phenylthiophene-3-carboxylic acid (20 mg) as a starting material.

¹H NMR (500 MHz, DMSO) δ 10.88 (s, 1H), 7.87 (d, J=7.6 Hz, 1H), 7.63-7.57 (m, 2H), 7.51 (s, 1H), 7.48-7.38 (m, 2H), 7.33-7.27 (m, 1H), 4.73 (ddd, J=12.7, 7.6, 5.6 Hz, 1H), 4.12 (s, 3H), 2.79 (ddd, J=17.3, 13.4, 5.8 Hz, 1H), 2.57-2.52 (m, 1H), 2.19-2.03 (m, 2H).

LCMS (m/z [M+H]⁺): 345.2

### Example 175: Synthesis of 5-(tert-butyl)-N-(2,6-dioxopiperidin-3-yl)-2-methoxythiophene-3-carboxamide (86)

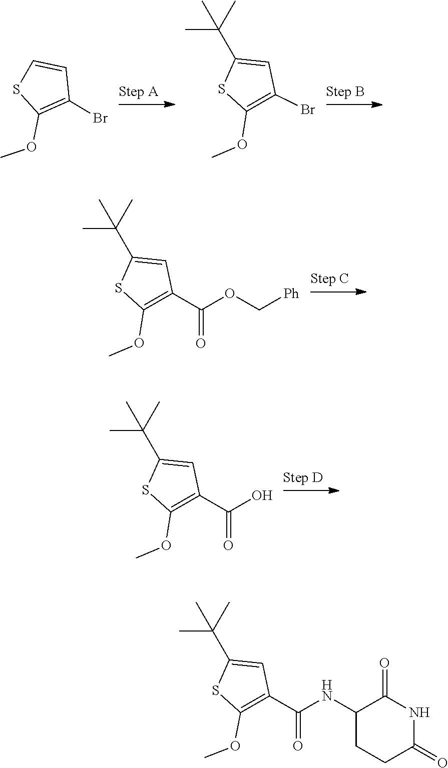

### Step A

To stirred solution of AlCl₃ (2.1 g, 15.544 mmol) in DCM (20 mL) at −78° C. was added tert-butyl bromide (1.9 g, 13.472 mmol) in DCM (10 mL) dropwise at −78° C. and stirred for 20 min. 3-bromo-2-methoxythiophene (2 g, 10.363 mmol) in DCM (10 mL) was added dropwise stirred for 2 h. The reaction mixture was warmed to RT and stirred for another 16 h. The reaction mixture was quenched with water and extracted with DCM, concentrated under reduced pressure and purified by flash column chromatography to give 3-bromo-5-(tert-butyl)-2-methoxythiophene (31% yield).

### Step B

To a stirred solution of 3-bromo-5-(tert-butyl)-2-methoxythiophene (900 mg, 3.614 mmol) in THE (22 mL) was added n-BuLi (1.8 M in THF) (4 ml, 7.229 mmol) dropwise at −78° C. Reaction mixture was stirred for 1 h at −78° C. and benzyl chloroformate (1.03 ml, 7.229 mmol) was added dropwise. The reaction was continued for 1 h, quenched with water, extracted with ethyl acetate and concentrated under reduced pressure. The product was purified by flash column chromatography to give benzyl 5-(tert-butyl)-2-methoxythiophene-3-carboxylate (220 mg, 20% yield) as light yellow oil.

### Step C

To a stirred solution of benzyl 5-(tert-butyl)-2-methoxythiophene-3-carboxylate (450 mg, 1.47 mmol) in THE (8 mL) and methanol (8 mL) at 5° C. was added 50% aq. NaOH (16 mL). The reaction mixture was stirred at RT for 16 h and acidified with 6 M HCl. The solids were filtered, washed with pentane and dried to give 5-(tert-butyl)-2-methoxythiophene-3-carboxylic acid (69% yield).

### Step D

5-(tert-butyl)-N-(2,6-dioxopiperidin-3-yl)-2-methoxythiophene-3-carboxamide was synthesized using the general procedure shown in Reaction Scheme 1 and Synthetic Conditions C, above, (75% yield) using 5-(tert-butyl)-2-methoxythiophene-3-carboxylic acid (20 mg) as a starting material.

¹H NMR (500 MHz, DMSO) δ 10.85 (s, 1H), 7.75 (d, J=7.5 Hz, 1H), 6.83 (s, 1H), 4.68 (ddd, J=12.2, 7.5, 5.7 Hz, 1H), 4.02 (s, 3H), 2.77 (ddd, J=17.3, 13.3, 6.0 Hz, 1H), 2.57-2.52 (m, 1H), 2.17-1.99 (m, 2H), 1.31 (s, 9H).

LCMS (m/z [M+H]⁺): 325.2

### Example 176: Synthesis of 2-amino-N-(2,6-dioxopiperidin-3-yl)thiophene-3-carboxamide (87)

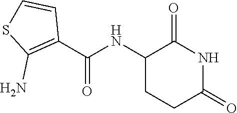 [SMILES: Nc1sccc1C(=O)NC1CCC(=O)NC1=O]

Tert-butyl (3-((2,6-dioxopiperidin-3-yl)carbamoyl)thiophen-2-yl)carbamate (1.0 g, 2.8 mmol) was dissolved in dichloromethane (10 mL) and a 10% solution of HCl in dioxane (3 mL) was added dropwise. The reaction mixture was stirred for 48 h at room temperature. The mixture was concentrated under reduced pressure and purified by preparative HPLC to give 2-amino-N-(2,6-dioxopiperidin-3-yl)thiophene-3-carboxamide (4% yield).

¹H NMR (500 MHz, DMSO) δ 10.78 (s, 1H), 7.96 (d, J=8.3 Hz, 1H), 7.22 (s, 2H), 7.07 (d, J=5.8 Hz, 1H), 6.28 (d, J=8.3 Hz, 1H), 4.69-4.61 (m, 1H), 2.82-2.68 (m, 1H), 2.57-2.52 (m, 1H), 2.15-2.04 (m, 1H), 1.96-1.84 (m, 1H).

LCMS (m/z [M+H]⁺): 254.0

### Example 177: Synthesis of 2-amino-5-chloro-N-(2,6-dioxopiperidin-3-yl)thiophene-3-carboxamide 89)

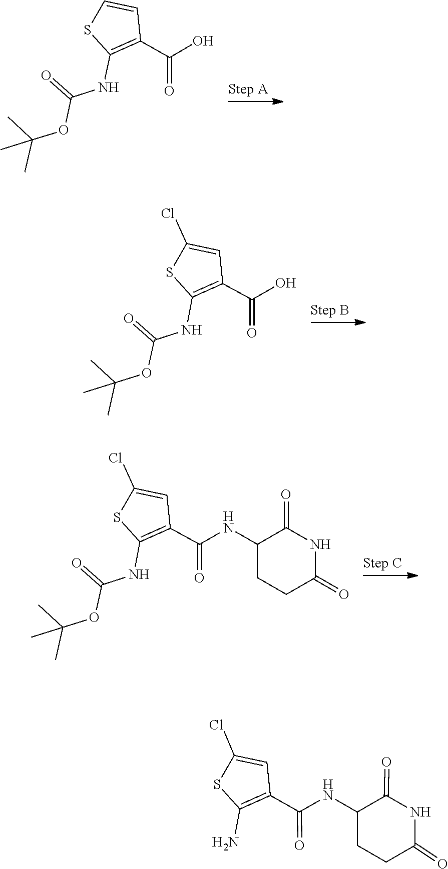

### Step A

N-chlorosuccinimide (2.2 g, 16.5 mmol) was added to a solution of 2-((tert-butoxycarbonyl)amino)thiophene-3-carboxylic acid (3.3 g, 13.6 mmol) in DMF (20 mL) and the reaction mixture was stirred at RT for 2 h. The mixture was diluted with water and filtered. The solids were washed with water and dried to give 2-{[(tert-butoxy)carbonyl]amino}-5-chlorothiophene-3-carboxylic acid (84% yield).

### Step B

Tert-butyl N-{5-chloro-3-[(2,6-dioxopiperidin-3-yl)carbamoyl]thiophen-2-yl}carbamate was synthesized using the general procedure shown in Reaction Scheme 1 and Synthetic Conditions D, above (81% yield), and 2-{[(tert-butoxy)carbonyl]amino}-5-chlorothiophene-3-carboxylic acid as a starting material.

### Step C

10% HCl in dioxane (2 mL) was added dropwise to a solution of the tert-butyl N-{5-chloro-3-[(2,6-dioxopiperidin-3-yl)carbamoyl]thiophen-2-yl}carbamate (2.0 g, 5.16 mmol) in dichloromethane (15 mL) and the mixture was stirred in ultrasonic bath for 8 h, concentrated under reduced pressure and purified by HPLC to give 2-amino-5-chloro-N-(2,6-dioxopiperidin-3-yl)thiophene-3-carboxamide (15% yield).

¹H NMR (400 MHz, DMSO) δ 10.81 (s, 1H), 7.97 (d, J=8.3 Hz, 1H), 7.41 (brs, 2H), 7.13 (s, 1H), 4.66-4.56 (m, 1H), 2.83-2.68 (m, 1H), 2.59-2.52 (m, 1H), 2.13-1.99 (m, 1H), 1.97-1.82 (m, 1H)

LCMS (m/z [M+H]⁺): 288.1

### Example 178: Synthesis of N-(2,6-dioxopiperidin-3-yl)-2-acetamido-5-(trifluoromethyl)thiophene-3-carboxamide (90)

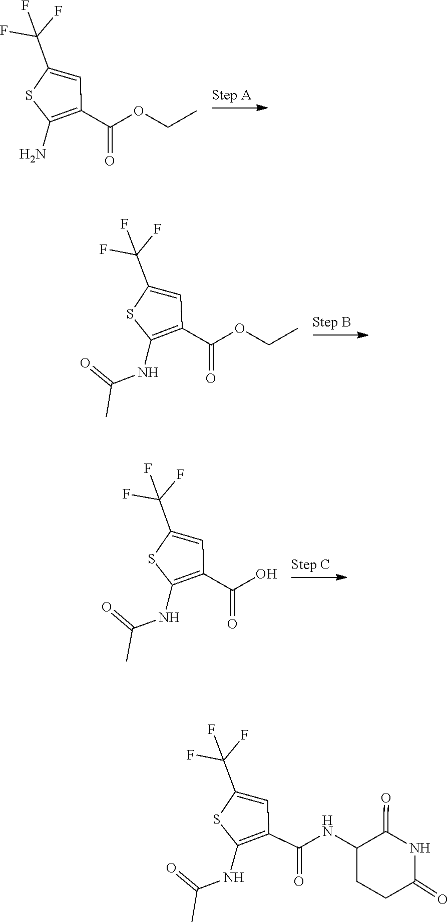 [SMILES: CCOC(=O)c1cc(C(F)(F)F)sc1N ; CCOC(=O)c1cc(C(F)(F)F)sc1NC(C)=O ; CC(=O)Nc1sc(C(F)(F)F)cc1C(=O)O ; CC(=O)Nc1sc(C(F)(F)F)cc1C(=O)NC1CCC(=O)NC1=O]

### Step A

Triethylamine (0.397 g, 3.92 mmol) and acetic anhydride (0.400 g, 3.92 mmol) were added to a solution of ethyl 2-amino-5-(trifluoromethyl)thiophene-3-carboxylate (0.852 g, 3.56 mmol) in MeCN (15 mL). The reaction mixture was stirred overnight at 50° C., cooled to rt, concentrated under reduced pressure, and extracted with DCM, dried over Na₂SO₄, and concentrated to give ethyl 2-acetamido-5-(trifluoromethyl)thiophene-3-carboxylate (91% yield).

### Step B

10% solution of LiOH (0.081 g, 3.4 mmol) was added to a solution of ethyl 2-acetamido-5-(trifluoromethyl)thiophene-3-carboxylate (0.911 g, 3.24 mmol) in THE (15 mL) and the resulting mixture was stirred for 5 days at RT. The solvents were evaporated under reduced pressure, the residue was diluted with water and washed with MTBE. The aqueous layer acidified by citric acid and the precipitate was filtered, washed with water, and dried to give 2-acetamido-5-(trifluoromethyl)thiophene-3-carboxylic acid (28% yield).

### Step C

N-(2,6-dioxopiperidin-3-yl)-2-acetamido-5-(trifluoromethyl)thiophene-3-carboxamide was synthesized using the general procedure shown in Reaction Scheme 1 and Synthetic Conditions D, above (26% yield), and 2-acetamido-5-(trifluoromethyl)thiophene-3-carboxylic acid as a starting material.

¹H NMR (400 MHz, DMSO) δ 11.98 (s, 1H), 10.93 (s, 1H), 9.00-8.81 (m, 1H), 8.13 (s, 1H), 4.87-4.62 (m, 1H), 2.98-2.65 (m, 2H), 2.28 (s, 3H), 2.20-2.08 (m, 1H), 2.07-1.88 (m, 1H).

LCMS (m/z [M+H]⁺): 364.2

### Example 179: Synthesis of 5-cyclopropyl-N-(2,6-dioxopiperidin-3-yl)-2-acetamidothiophene-3-carboxamide (91)

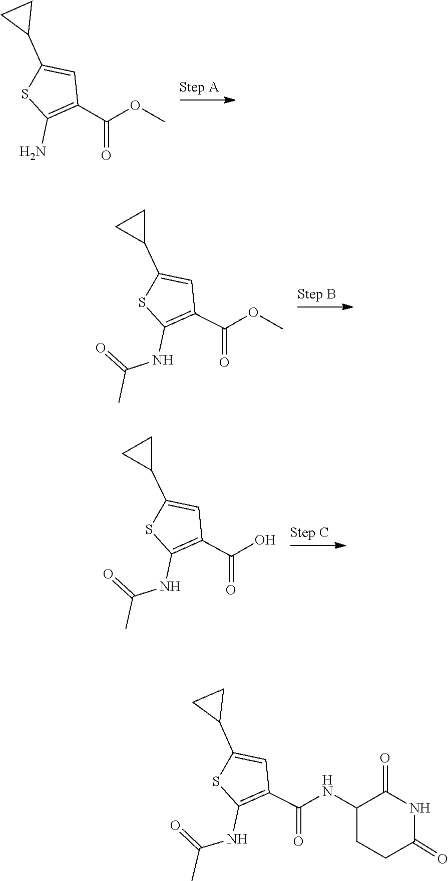 [SMILES: COC(=O)c1cc(C2CC2)sc1N ; COC(=O)c1cc(C2CC2)sc1NC(C)=O ; CC(=O)Nc1sc(C2CC2)cc1C(=O)O ; CC(=O)Nc1sc(C2CC2)cc1C(=O)NC1CCC(=O)NC1=O]

### Step A

methyl 5-cyclopropyl-2-acetamidothiophene-3-carboxylate was synthesized in 69% yield using Example Method 3, above, using 2-amino-5-cyclopropylthiophene-3-carboxylate as a starting material.

### Step B

5-cyclopropyl-2-acetamidothiophene-3-carboxylic acid was synthesized in 57% yield using Example Method 2, above, and methyl 5-cyclopropyl-2-acetamidothiophene-3-carboxylate as a starting material.

### Step C

5-cyclopropyl-N-(2,6-dioxopiperidin-3-yl)-2-acetamidothiophene-3-carboxamide was synthesized using the general procedure shown in Reaction Scheme 1 and Synthetic Conditions E, above (56% yield), and 5-cyclopropyl-2-acetamidothiophene-3-carboxylic acid as a starting material.

¹H NMR (400 MHz, DMSO) δ 11.73 (s, 1H), 10.89 (s, 1H), 8.52 (d, J=8.5 Hz, 1H), 7.09 (s, 1H), 4.76-4.64 (m, 1H), 2.85-2.71 (m, 1H), 2.62-2.53 (m, 1H), 2.22-2.04 (m, 4H), 2.04-1.89 (m, 2H), 1.00-0.87 (m, 2H), 0.68-0.57 (m, 2H)

LCMS (m/z [M+H]⁺): 336.2

### Example 180: Synthesis of 5-chloro-N-(2,6-dioxopiperidin-3-yl)-2-(2-phenylacetamido)thiophene-3-carboxamide (92)

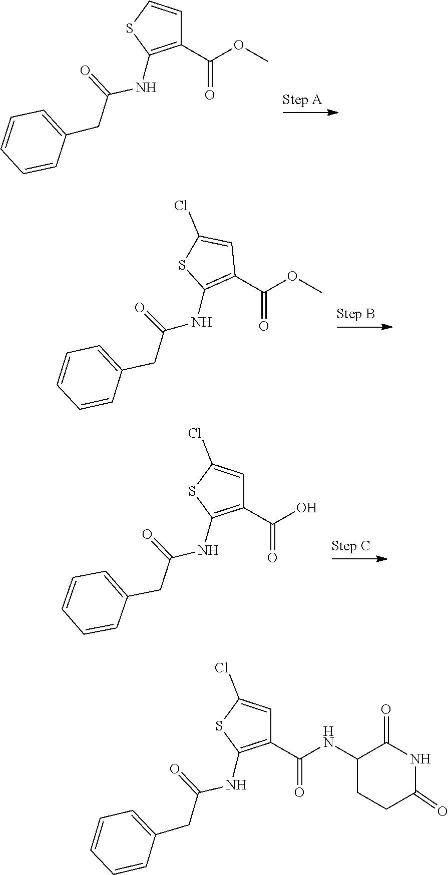 [SMILES: COC(=O)c1ccsc1NC(=O)Cc1ccccc1 ; COC(=O)c1cc(Cl)sc1NC(=O)Cc1ccccc1 ; O=C(Cc1ccccc1)Nc1sc(Cl)cc1C(=O)O ; O=C1CCC(NC(=O)c2cc(Cl)sc2NC(=O)Cc2ccccc2)C(=O)N1]

### Step A

Methyl 5-chloro-2-(2-phenylacetamido)thiophene-3-carboxylate was synthesized using Example Method 1, above (75% yield), using methyl 2-(2-phenylacetamido)thiophene-3-carboxylate as a starting material.

### Step B

5-chloro-2-(2-phenylacetamido)thiophene-3-carboxylic acid was synthesized using Example Method 2, above (82% yield), using methyl 5-chloro-2-(2-phenylacetamido)thiophene-3-carboxylate as a starting material.

### Step C

5-chloro-N-(2,6-dioxopiperidin-3-yl)-2-(2-phenylacetamido)thiophene-3-carboxamide was synthesized using the general procedure shown in Reaction Scheme 1 and Synthetic Conditions D, above (15% yield), with 5-chloro-2-(2-phenylacetamido)thiophene-3-carboxylic acid as a starting material.

¹H NMR (400 MHz, DMSO) δ 11.94 (s, 1H), 10.91 (s, 1H), 8.72-8.54 (m, 1H), 7.66-7.45 (m, 2H), 7.43-7.21 (m, 4H), 4.82-4.64 (m, 1H), 3.90 (s, 2H), 2.89-2.71 (m, 1H), 2.61-2.53 (m, 1H), 2.17-2.02 (m, 1H), 2.01-1.88 (m, 1H)

LCMS (m/z [M+H]⁺): 406.2

### Example 181: Synthesis of N-(2,6-dioxopiperidin-3-yl)-5-methoxythiazole-4-carboxamide (100)

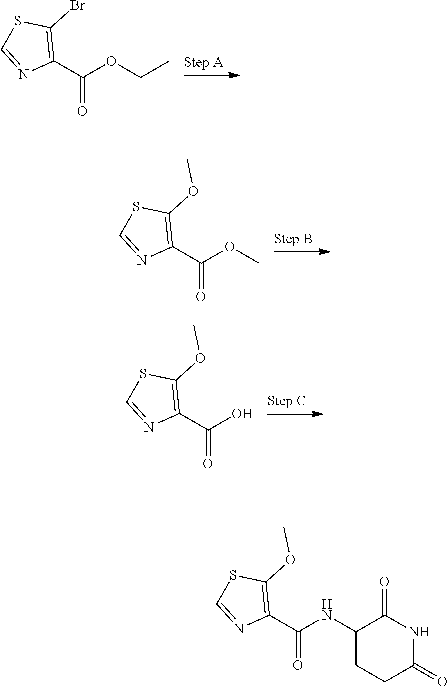

### Step A

To a stirred solution of ethyl 5-bromothiazole-4-carboxylate (2.0 g, 8.475 mmol, 1eq) in methanol (24 mL) was added NaOMe (25% in MeOH) (3.8 ml, 16.95 mmol, 2eq). The reaction mixture was refluxed for 2 h, cooled to RT and quenched by saturated ammonium chloride solution (10 mL).

The mixture was concentrated under reduced pressure and purified by flash column chromatography to give methyl 5-methoxythiazole-4-carboxylate (27% yield).

### Step B

To a stirring solution of methyl 5-methoxythiazole-4-carboxylate (100 mg, 0.578 mmol, 1eq) in a solution of THF, MeOH, H₂O (4:2:1) (7 mL) was added LiOH, H₂O (73 mg, 1.734 mmol, 3eq). The reaction mixture was stirred at RT for 16 h, evaporated, redissolved in water and washed with ethyl acetate. The aqueous layer was acidified by 0.5 M HCl, extracted with 10% MeOH in DCM, dried over Na₂SO₄, concentrated under reduced pressure and purified by flash column chromatography to give 5-methoxythiazole-4-carboxylic acid (32% yield).

### Step C

N-(2,6-dioxopiperidin-3-yl)-5-methoxythiazole-4-carboxamide was synthesized using the general procedure shown in Reaction Scheme 1 and Synthetic Conditions C, above, (9% yield) using 5-methoxythiazole-4-carboxylic acid (20 mg) as a starting material.

¹H NMR (500 MHz, DMSO) δ 10.81 (s, 1H), 8.51 (s, 1H), 8.23 (d, J=8.2 Hz, 1H), 4.67 (ddd, J=12.5, 8.2, 5.3 Hz, 1H), 4.04 (s, 3H), 2.83-2.73 (m, 1H), 2.52 (dt, J=3.9, 2.5 Hz, 1H), 2.19-2.09 (m, 1H), 1.97 (dtd, J=12.7, 5.5, 2.6 Hz, 1H).

LCMS (m/z [M+H]⁺): 269.8

### Example 182: Synthesis of 2-amino-N-(2,6-dioxopiperidin-3-yl)-5-methoxythiazole-4-carboxamide (101)

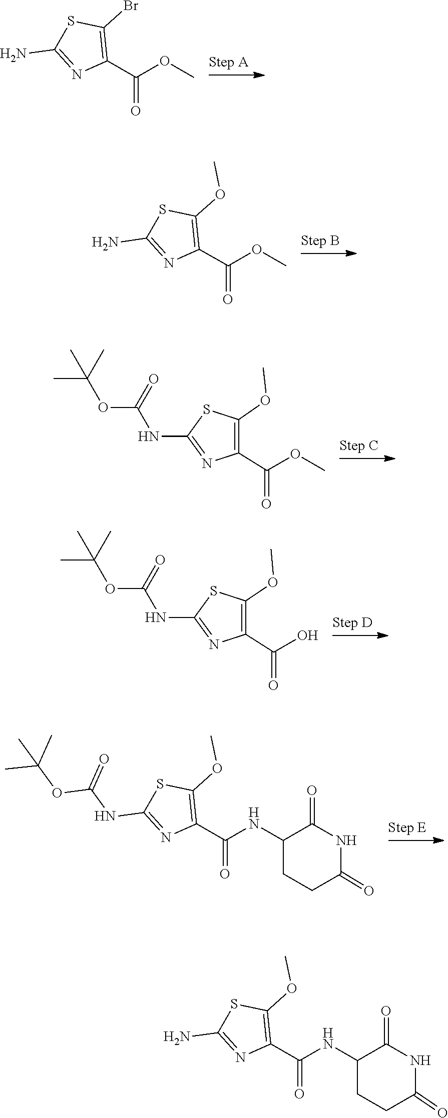

### Step A

To a stirring solution of methyl 2-amino-5-bromothiazole-4-carboxylate (1 g, 4.255 mmol, 1eq) in methanol (30 mL) was added NaOMe (25% in MeOH) (2.3 ml, 10.638 mmol, 2.5 eq). The reaction mixture was refluxed for 1.5 h, cooled to RT and quenched by saturated ammonium chloride solution (10 mL). The mixture was concentrated under reduced pressure and purified by flash column chromatography to give methyl 2-amino-5-methoxythiazole-4-carboxylate (50% yield).

### Step B

Methyl 2-amino-5-methoxythiazole-4-carboxylate (400 mg, 2.128 mmol. 1eq.) was dissolved in DCM then were added triethylamine (0.532 mmol, 2eq.) and Boc₂O (0.532 mmol, 2 eq). The reaction mixture was stirred at RTfor 18 h, diluted with DCM and washed successively with water and brine, dried over Na₂SO₄, concentrated under reduced pressure and purified by flash column chromatography to give methyl 2-((tert-butoxycarbonyl)amino)-5-methoxythiazole-4-carboxylate (49% yield).

### Step C

To a stirring solution of methyl 2-((tert-butoxycarbonyl)amino)-5-methoxythiazole-4-carboxylate (300 mg, 1.042 mmol, 1eq) in THF:MeOH:H₂O 3:2:1 (12 mL) was added LiOH·H₂O (131 mg, 3.125 mmol, 3eq). The reaction mixture was stirred at RT for 16 h, evaporated, redissolved in water and washed with ethyl acetate. The aqueous layer was acidified by 0.5 M HCl, extracted with 10% MeOH in DCM, dried over Na₂SO₄, concentrated under reduced pressure and triturated with ether and pentane to give 2-((tert-butoxycarbonyl)amino)-5-methoxythiazole-4-carboxylic acid (49% yield).

### Step D

Tert-butyl (4-((2,6-dioxopiperidin-3-yl)carbamoyl)-5-methoxythiazol-2-yl)carbamate was synthesized using the general procedure shown in Reaction Scheme 1 and Synthetic Conditions F, above (50% yield), and 2-((tert-butoxycarbonyl)amino)-5-methoxythiazole-4-carboxylic acid (20 mg) as a starting material.

### Step E

To a solution of tert-butyl (4-((2,6-dioxopiperidin-3-yl)carbamoyl)-5-methoxythiazol-2-yl)carbamate (19.6 mg, 0.051 mmol, 1 eq.) in water (3 mL) and dioxane (3 mL) was added 36% HCl (1.5 mL). The reaction was stirred at RT for 3 h and concentrated under reduced pressure to give 2-amino-N-(2,6-dioxopiperidin-3-yl)-5-methoxythiazole-4-carboxamide hydrochloride (100% yield).

¹H NMR (500 MHz, DMSO) δ 10.84 (s, 1H), 7.75 (d, J=7.8 Hz, 1H), 7.41-6.65 (m, 2H), 4.63 (ddd, J=12.0, 7.8, 5.8 Hz, 1H), 3.89 (s, 3H), 2.75 (ddd, J=17.3, 13.1, 6.2 Hz, 1H), 2.60-2.52 (m, 1H), 2.11-1.98 (m, 2H).

LCMS (m/z [M+H]⁺): 285.0

### Example 183: Synthesis of 2-amino-N-(2,6-dioxopiperidin-3-yl)thiazole-5-carboxamide (102)

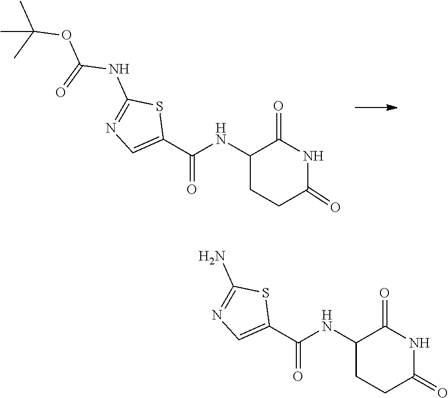 [SMILES: CC(C)(C)OC(=O)Nc1ncc(C(=O)NC2CCC(=O)NC2=O)s1 ; Nc1ncc(C(=O)NC2CCC(=O)NC2=O)s1]

To the suspension of tert-butyl (5-((2,6-dioxopiperidin-3-yl)carbamoyl)thiazol-2-yl)carbamate (71, 30 mg, 0.085 mmol, 1 eq) in DCM (1.5 mL) was added TFA (0.2 mL) and mixture was stirred for 18 h at RT, concentrated under reduced pressure and purified by HPLC to give 2-amino-N-(2,6-dioxopiperidin-3-yl)thiazole-5-carboxamide (yield 37%).

¹H NMR (500 MHz, DMSO) δ 10.81 (s, 1H), 8.35 (d, J=8.4 Hz, 1H), 7.64 (s, 1H), 7.49 (s, 2H), 4.64 (ddd, J=12.5, 8.4, 5.4 Hz, 1H), 2.76 (ddd, J=17.4, 13.3, 5.6 Hz, 1H), 2.53-2.51 (m, 2H), 2.05 (qd, J=12.8, 4.3 Hz, 1H), 1.93 (dddd, J=10.7, 8.1, 5.3, 2.9 Hz, 1H).

LCMS (m/z [M+H]⁺): 255.2

### Synthesis of Ligase Ligand Moieties—Compounds of Formula (IIa) and (IIb)

### Example 184: Synthesis of 7-bromo-N-(2,6-dioxopiperidin-3-yl)thieno[3,4-b]pyridine-5-carboxamide (103)

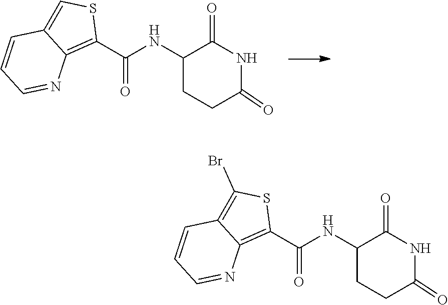 [SMILES: O=C1CCC(NC(=O)c2scc3cccnc23)C(=O)N1 ; O=C1CCC(NC(=O)c2sc(Br)c3cccnc23)C(=O)N1]

N-bromosuccinimide (96.8 mg, 0.544 mmol, 1.1 eq) was added to a suspension of N-(2,6-dioxopiperidin-3-yl)thieno[3,4-b]pyridine-7-carboxamide (143.0 mg, 0.494 mmol, 1.000 eq,) in DMF (4.9 mL) at ambient temperature. The reaction mixture was heated to 60° C. and stirred for 3 h. The obtained crude compound was purified by HPLC to give 5-bromo-N-(2,6-dioxopiperidin-3-yl)thieno[3,4-b]pyridine-7-carboxamide (15% yield).

¹H NMR (500 MHz, DMSO) δ 10.97 (s, 1H), 9.53 (d, J=7.4 Hz, 1H), 8.87 (dd, J=4.0, 1.5 Hz, 1H), 8.11 (dd, J=8.9, 1.5 Hz, 1H), 7.41 (dd, J=8.9, 4.0 Hz, 1H), 4.91 (ddd, J=12.8, 7.3, 5.6 Hz, 1H), 2.84 (ddd, J=17.5, 13.4, 5.7 Hz, 1H), 2.57 (ddd, J=17.4, 4.3, 2.3 Hz, 1H), 2.30-2.13 (m, 2H)

LCMS (m/z [M+H]⁺): 368.37

### Example 185: Synthesis of 4-chloro-N-(2,6-dioxopiperidin-3-yl)-1H-pyrrolo[2,3-b]pyridine-3-carboxamide (104)

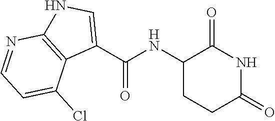 [SMILES: O=C1CCC(NC(=O)c2c[nH]c3nccc(Cl)c23)C(=O)N1]

This compound was synthesized using the general procedure shown in Reaction Scheme 1 and Synthetic Conditions C, above, (15% yield) using 4-chloro-1H-pyrrolo[2,3-b]pyridine-3-carboxylic acid (20 mg) as a starting material.

¹H NMR (500 MHz, DMSO) δ 12.40 (s, 1H), 10.82 (s, 1H), 8.38 (d, J=8.3 Hz, 1H), 8.22 (d, J=5.1 Hz, 1H), 7.96 (s, 1H), 7.25 (d, J=5.1 Hz, 1H), 4.79-4.72 (m, 1H), 2.79 (ddd, J=17.9, 9.7, 7.0 Hz, 1H), 2.59-2.52 (m, 1H), 2.12-2.01 (m, 2H).

LCMS (m/z [M+H]⁺): 306.9

### Example 186: Synthesis of 5-chloro-N-(2,6-dioxopiperidin-3-yl)-1H-pyrrolo[2,3-b]pyridine-3-carboxamide (105)

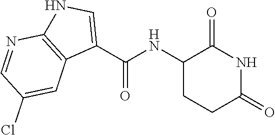 [SMILES: O=C1CCC(NC(=O)c2c[nH]c3ncc(Cl)cc23)C(=O)N1]

This compound was synthesized using the general procedure shown in Reaction Scheme 1 and Synthetic Conditions C, above, (31% yield) using 5-chloro-1H-pyrrolo[2,3-b]pyridine-3-carboxylic acid (20 mg) as a starting material.

¹H NMR (500 MHz, DMSO) δ 12.40 (s, 1H), 10.85 (s, 1H), 8.44 (d, J=2.5 Hz, 1H), 8.39 (d, J=8.4 Hz, 1H), 8.30 (d, J=2.4 Hz, 1H), 8.27 (d, J=2.9 Hz, 1H), 4.79 (ddd, J=12.2, 8.3, 5.3 Hz, 1H), 2.81 (ddd, J=17.3, 13.2, 5.5 Hz, 1H), 2.59-2.52 (m, 1H), 2.11 (qd, J=12.8, 4.4 Hz, 1H), 2.01 (dtd, J=13.0, 5.4, 2.9 Hz, 1H).

LCMS (m/z [M+H]⁺): 307.2

### Example 187: Synthesis of 5-chloro-N-(2,6-dioxopiperidin-3-yl)thieno[3,4-b]pyridine-7-carboxamide (106)

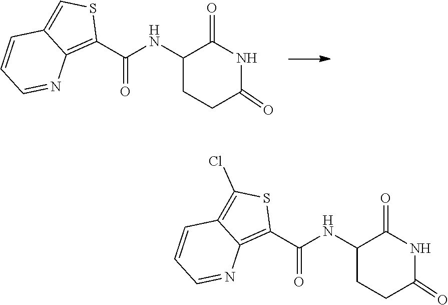 [SMILES: O=C1CCC(NC(=O)c2scc3cccnc23)C(=O)N1 ; O=C1CCC(NC(=O)c2sc(Cl)c3cccnc23)C(=O)N1]

N-chlorosuccinimide (0.059 g, 0.442 mmol, 1.1 eq) was added to a suspension of N-(2,6-dioxopiperidin-3-yl)thieno[3,4-b]pyridine-7-carboxamide (0.116 g, 0.401 mmol) in DMF (5 mL) at RT. The reaction mixture was heated to 60° C. and stirred for 3 h. The obtained crude compound was purified by HPLC to give 5-chloro-N-(2,6-dioxopiperidin-3-yl)thieno[3,4-b]pyridine-7-carboxamide (43% yield).

¹H NMR (400 MHz, DMSO) δ 10.97 (s, 1H), 9.51 (d, J=7.4 Hz, 1H), 8.91-8.83 (m, 1H), 8.19 (d, J=8.8 Hz, 1H), 7.41-7.33 (m, 1H), 4.95-4.84 (m, 1H), 2.89-2.74 (m, 1H), 2.62-2.55 (m, 1H), 2.28-2.11 (m, 2H)

LCMS (m/z [M+H]⁺): 323.8

### Example 188: Synthesis of N-(2,6-dioxopiperidin-3-yl)thieno[3,4-b]pyridine-7-carboxamide (107)

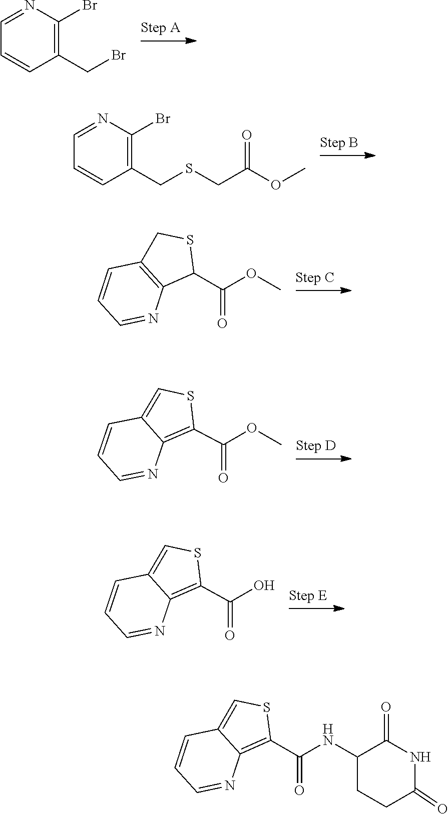 [SMILES: BrCc1cccnc1Br ; COC(=O)CSCc1cccnc1Br ; COC(=O)C1SCc2cccnc21 ; COC(=O)c1scc2cccnc12 ; O=C(O)c1scc2cccnc12 ; O=C1CCC(NC(=O)c2scc3cccnc23)C(=O)N1]

### Step A

To an ice-cold solution of 2-bromo-3-(bromomethyl)pyridine 2 (10.5 g, 42.0 mmol) in THE (100 mL) was added methyl thioglycolate (4.089 g, 18.124 mmol) followed by Et₃N under stirring. The mixture was warmed to RT and stirred for further 30 min. The reaction mixture was diluted with water and extracted with DCM, dried over Na₂SO₄, concentrated under reduced pressure and purified by flash column chromatography to give methyl 2-{[(2-bromopyridin-3-yl)methyl]sulfanyl}acetate (53% yield).

### Step B

A solution of methyl 2-{[(2-bromopyridin-3-yl)methyl]sulfanyl}acetate (4.5 g, 16.295 mmol) in THE (25 mL) was added slowly to a suspension of KH (1.307 g, 32.591 mmol) and stirred for 20 min at room temperature. The reaction mixture was then cooled to −78° C. and treated with saturated aqueous NH₄Cl solution, warmed to RT, extracted with DCM, dried over Na₂SO₄, concentrated under reduced pressure and purified by flash column chromatography to give methyl 5H,7H-thieno[3,4-b]pyridine-7-carboxylate (56% yield).

### Step C

To the stirred solution of methyl 5H,7H-thieno[3,4-b]pyridine-7-carboxylate (3 g, 15.385 mmol) in CHCl₃ (25 mL) was added activated MnO₂ (13.375 g, 153.846 mmol) and the reaction mixture was stirred at RT for 16 h, filtered through celite bed, concentrated under reduced pressure and purified by flash column chromatography to give methyl thieno[3,4-b]pyridine-7-carboxylate (46% yield).

### Step D

To a stirred solution of methyl thieno[3,4-b]pyridine-7-carboxylate (1.5 g, 7.772 mmol) in THF:MeOH:H₂O, 4:2:1 (14 mL) was added LiOH·H₂O (1.304 g, 31.088 mmol) at 0° C. and then ice-bath was removed and the mixture was stirred at RT for 2.5 h. Saturated aqueous citric acid solution was added and the product was extracted with 10% MeOH in DCM, dried over Na₂SO₄, concentrated under reduced pressure and purified by HPLC to give thieno[3,4-b]pyridine-7-carboxylic acid (72 mg, 5%).

### Step E

Synthesis of N-(2,6-dioxopiperidin-3-yl)thieno[3,4-b]pyridine-7-carboxamide was synthesized using the general procedure shown in Reaction Scheme 1 and Synthetic Conditions C, above (69% yield), and thieno[3,4-b]pyridine-7-carboxylic acid (25.0 mg) as a starting material.

¹H NMR (500 MHz, DMSO) δ 10.95 (s, 1H), 9.71 (d, J=7.4 Hz, 1H), 8.83 (dd, J=4.0, 1.6 Hz, 1H), 8.59 (s, 1H), 8.32 (dd, J=8.8, 1.6 Hz, 1H), 7.29 (dd, J=8.7, 4.0 Hz, 1H), 4.92 (ddd, J=12.7, 7.4, 5.4 Hz, 1H), 2.89-2.79 (m, 1H), 2.60-2.52 (m, 1H), 2.30-2.11 (m, 2H).

LCMS (m/z [M+H]⁺): 290.0

### Complex Formation and Protein Degradation Examples

### Example 189: Ternary Complex Formation Assay

The effect of the bifunctional compounds of the invention on the formation of a ternary complex composed of [MCL-1]-[compound of formula (I)]-[CRBN/DDB1] was investigated with two methods: AlphaLISA dose response assay or HTRF ternary complex assay.

### AlphaLISA Dose Response Assay:

Two types of protein solution were prepared:
40 nM biotinylated hMCL-1, 40 μg/ml AlphaScreen Streptavidin-coated Donor Beads in PBS buffer with 0.1% Tween-20 and 1 mM DTT,
200 nM 6×His-CRBN/Strep-DDB1, 40 μg/ml AlphaLISA Anti-6×His Acceptor beads in PBS buffer with 0.1% Tween-20 and 1 mM DTT.

The prepared solutions were incubated at room temperature for 45 min and then the solution containing the donor beads was mixed with the solution containing the acceptor beads.

The tested compounds were dispensed onto a white 384-well AlphaPlate 384 SW. DMSO was backfilled to all wells, resulting in a final DMSO content of 2%. Wells containing only DMSO served as background. Next, 10 μl of solution with donor and acceptor beads was added to the wells.

The plate was sealed with transparent film and shaken using a VibroTurbulator for 60 see at room temperature, level 3. The plate was then spun down shortly (10 s, 1000 rcf, room temperature) and incubated at 25° C. for 30 min.

The read-out was performed with PerkinElmer Enspire Multimode Plate Reader (method for AlphaLISA 384-well low volume, Filterset: λexc=680 nm, λem=615 nm).

The results were analyzed as follows:
1) an average of luminescence for background signal was calculated and used as a negative control;
2) average of the maximum measured luminescence for 233 was calculated and used as an internal positive control;
3) raw luminescence values were normalized against positive and negative controls;
4) concentration points above the hook point were excluded;
5) EC50 and pEC50 values were determined.

As illustrated in Table 8, the compounds of the present invention have the capability to induce the formation of the [MCL-1]-[compound of formula (I)]-[CRBN/DDB1] complex.

### HTRF Ternary Complex Assay:

The effect of the bifunctional compounds of the invention on the formation of a ternary complex composed of [MCL-1]-[compound of formula (I)]-[CRBN/DDB1] was investigated.

Mix of protein solution were prepared:
48 nM hMcl-1 and 105.6 nM CRBN was prepared in PPI Europium detection buffer with 2 mM DTT.
Acceptor and donor stock solutions were diluted 100 times in PPI Europium detection buffer. Equal volumes of both reagents were mixed. Finally giving 3 nM of Streptavidin-Eu cryptate (acceptor) and 6.67 nM of Anti-6× his-d2 (donor).

5 μl of protein mix was dispensed to all wells. The tested compounds in dose-response were dispensed onto a white 384-well low volume plate (Greiner, 784075). DMSO was backfilled to all wells, resulting in a final DMSO content of 0.5%. Wells containing only DMSO served as background. Next, 5 μl of solution with donor and acceptor was added to the wells.

The plate was sealed with transparent film and shaken using a VibroTurbulator for 60 see at level 3. The plate was then spun down shortly (10 s, 1000 rcf) and incubated at 25° C. for 180 min.

The read-out was performed with plate reader (Pherastar, BMG Labtech) in time resolved fluorescence mode. Filterset: TR 337 665 620.

The results were analyzed as follows:
1) an average of fluorescence for background signal was calculated and used as a negative control;
2) raw fluorescence values for tested compounds were normalized against negative controls;
3) bell shape curve was fitted to a gaussian distribution using a non-linear least squares Gauss-Newton algorithm;
4) EC50 and pEC50 values were determined.

As illustrated in Table 8, the compounds of the present invention have the capability to induce the formation of the [MCL-1]-[compound of formula (I)]-[CRBN/DDB1] complex.

TABLE 8
Ternary complex assay results for the compounds of the invention

| | AlphaLISA dose response | HTRF ternary complex |
|---|---|---|
| | Average pEC50 | Average pEC50 |
| Compound | by molecule | by molecule |
| 201 | >7 | |
| 203 | >7 | |
| 204 | >7 | |
| 206 | >7 | |
| 207 | >7 | |
| 208 | >7 | |
| 209 | >7 | |
| 210 | >7 | |
| 211 | >7 | |
| 213 | >7 | |
| 214 | >8 | |
| 215 | >7 | |
| 216 | >8 | >8 |
| 217 | >7 | |
| 218 | >7 | |
| 219 | >8 | |
| 220 | >7 | >7 |
| 221 | >7 | >7 |
| 222 | >7 | |
| 223 | >7 | |
| 224 | >7 | |
| 225 | >7 | |
| 226 | >7 | |
| 227 | >6 | |
| 232 | >6 | |
| 233 | >6 | |
| 241 | >7 | |
| 245 | >6 | |
| 247 | >6 | |
| 248 | >6 | |
| 249 | >7 | |
| 251 | >7 | |
| 252 | | >7 |
| 261 | >6 | |
| 263 | >7 | |
| 266 | >7 | |
| 267 | >7 | |
| 268 | | >8 |
| 292 | >8 | |
| 294 | >7 | |
| 295 | >7 | >7 |
| 296 | >7 | |
| 298 | >8 | |
| 299 | | >7 |
| 300 | >8 | |
| 301 | >8 | >8 |
| 302 | | >8 |
| 303 | | >7 |
| 304 | | >7 |
| 305 | | >7 |
| 306 | | >8 |
| 308 | >7 | >7 |
| 311 | | >7 |
| 315 | >7 | |
| 316 | >6 | |
| 317 | >7 | |
| 318 | >7 | |
| 319 | >7 | |
| 321 | >8 | >7 |
| 322 | | >7 |
| 325 | >8 | |
| 326 | >7 | >8 |
| 327 | >7 | >8 |
| 328 | >7 | >7 |
| 329 | >7 | |
| 330 | >7 | >7 |
| 331 | >7 | |
| 332 | >7 | |
| 333 | | >8 |
| 334 | | >8 |
| 335 | | >8 |
| 336 | | >8 |
| 337 | | >8 |
| 338 | | >8 |
| 342 | >7 | |
| 345 | >7 | |
| 269 | >7 | >8 |
| 270 | >7 | >8 |
| 271 | >7 | |
| 272 | >7 | |
| 273 | >8 | |
| 274 | >8 | |
| 275 | >7 | |
| 276 | >7 | |
| 277 | >7 | |
| 278 | >7 | |
| 280 | >8 | >8 |
| 281 | >7 | >8 |
| 282 | | >7 |
| 284 | >7 | |
| 285 | | >8 |
| 288 | | >7 |
| 290 | | >8 |
| 291 | | >7 |
| 346 | >7 | |
| 347 | | >7 |
| 348 | | >7 |
| 349 | | >7 |
| 351 | >8 | |
| 352 | >6 | |
| 353 | >6 | |
| 354 | >7 | >7 |

### Example 190: OPM-2 Cells: MCL-1Protein Degradation, Western Blot

The effect of various compounds of the invention and the reference compounds on MCL-1 protein degradation in the OPM-2 cell line was investigated, using the degradation assay protocol below.

OPM-2 cells were maintained in RPMI-1640 medium, supplemented with penicillin/streptomycin and 10% Fetal Bovine Serum (FBS). During the treatment, FBS concentration was reduced to 1%.

Compound stocks were pre-diluted in DMSO and added directly to the cells growth medium (final DMSO concentration: 0.5%).

OPM-2 cells were collected, centrifuged (250 rcf, 5 min), resuspended in a growth medium containing 1% FBS and counted. The cell suspension was then adjusted to 1×10⁶ cell/ml using a growth medium containing 1% FBS. Afterwards, 0.5×10⁶ cells were seeded on 24-well plates in 0.5 mL of medium per well. Immediately after seeding cells, compounds in appropriate concentrations were added to the cell culture medium.

Following the treatment with the compounds, cells were collected from the plates and suspended in a lysis buffer (2% SDS, 50 mM Tris, pH 8.0, 100 U/ml Pierce Universal Nuclease for Cell Lysis, complete EDTA-free Protease Inhibitor Cocktail) and incubated on ice for 45 min. The amount of protein was determined via BCA assay, and the equal quantity of each sample was loaded on the precast gel for the protein separation. Following the electrophoresis and transfer to the membrane, the membrane was stained with Ponceau (0.1% (w/v) Ponceau in 5% acetic acid). After total protein detection, the stain was washed out using TBST. After the antibody staining for MCL-1 and α-tubulin, the membranes were washed and signals developed.

Densitometric values for MCL-1 were normalized to total protein level (the Ponceau Staining). [%] of protein reduction is calculated in comparison to the cells treated with DMSO, which stand for 0%.

The results for the 6 h treatment with 100 nM compounds are shown in Table 9, using the following labels:
<30% for 0-29% of MCL-1 protein reduction,
≥30% for 30-49% of MCL-1 protein reduction,
≥50% for 50-100% of MCL-1 protein reduction.

As illustrated in Table 9, the compounds of the present invention have the capability to induce degradation of MCL-1 protein with improved potency as compared to the known compound dMCL1-2.

### Example 191: OPM-2 Cells: MCL-1 Protein Degradation, Simple Western

The effect of various compounds of the invention and the reference compounds on MCL-1protein degradation in the OPM-2 cell line was investigated, using the degradation assay protocol below.

OPM-2 cells were maintained in RPMI-1640 medium, supplemented with penicillin/streptomycin and 10% Fetal Bovine Serum (FBS). During the treatment, FBS concentration was reduced to 1%.

Compound stocks were pre-diluted in DMSO and added directly to the cells growth medium (final DMSO concentration: 0.5%).

OPM-2 cells were collected, centrifuged (250 rcf, 5 min), resuspended in a growth medium containing 1% FBS and counted. The cell suspension was then adjusted to 1×106 cell/ml using a growth medium containing 1% FBS. Afterwards, 0.5×106 cells were seeded on 24-well plates in 0.5 mL of medium per well. Immediately after seeding cells, compounds in appropriate concentrations were added to the cell culture medium.

Following the treatment with the compounds, cells were collected from the plates and suspended in a lysis buffer (2% SDS, 50 mM Tris, pH 8.0, 100 U/ml Pierce Universal Nuclease for Cell Lysis, cOmplete EDTA-free Protease Inhibitor Cocktail) and incubated on ice for 45 min.

The following steps, including sample loading, protein separation, immunoprobing or labelling, washes and detection analysis was performed using Jess from ProteinSimple, an automatic system for analyzing protein levels in cell lysates (12-230 kDa Jess or Wes separation module 8×25 capillaries, protein normalization assay, no. SM-PN01-1). The Protein of Interest was detected using MCL-1 (D35A5) Rabbit monoclonal antibody (Cell Signaling, #5453, LOT: 4). Antibody was diluted in a ratio of 1:100 in Antibody diluent.

[%] of protein reduction is calculated in comparison to the cells treated with DMSO, which stand for 0%.

The results for the 6 h treatment with 100 nM compounds are shown in Table 9, using the following labels:
<20% for 0-19% of MCL-1 protein reduction,
≥20% for 20-39% of MCL-1 protein reduction,
≥40% for 40-100% of MCL-1 protein reduction.

As illustrated in Table 9, the compounds of the present invention have the capability to induce degradation of MCL-1 protein with improved potency as compared to the known compound dMCL1-2.

TABLE 9
MCL-1 protein degradation results for the compounds
of the invention and the reference compound

OPM-2 cell line

| | Western blot, | Simple Western, |
|---|---|---|
| Compound | % of protein reduction | % of protein reduction |
| 201 | ≥50 | ≥20 |
| 204 | ≥50 | ≥40 |
| 206 | ≥50 | ≥40 |
| 213 | — | ≥40 |
| 214 | ≥50 | ≥40 |
| 215 | ≥50 | ≥20 |
| 217 | — | ≥40 |
| 220 | ≥50 | — |
| 241 | ≥50 | ≥20 |
| 245 | — | ≥20 |
| 249 | — | ≥20 |
| 260 | — | ≥20 |
| 263 | ≥50 | ≥40 |
| 264 | — | ≥20 |
| 266 | — | ≥40 |
| 268 | ≥50 | — |
| 346 | ≥50 | — |
| dMCL1-2 | <30% | <20% |

### Example 192: DMS 114 Cells: MCL-1 Protein Degradation, Western Blot

The effect of various compounds of the invention on MCL-1 protein degradation in the DMS 114 cell line was investigated, using the degradation assay protocol below.

DMS 114 cells were maintained in Waymouth's MB 752/1 medium, supplemented with penicillin/streptomycin and 10% Fetal Bovine Serum (FBS).

Compound stocks were pre-diluted in DMSO and added directly to the cells growth medium (final DMSO concentration: 0.5%).

DMS 114 cells were detached, collected, centrifuged (250 rcf, 5 min), resuspended in growth medium containing 10% FBS and counted. Cell suspension was then adjusted to 0.25×10⁶ cell/ml using growth medium containing 10% FBS. Afterwards, 0.5×10⁶ cells were seeded on 6-well plates in 2 mL of medium per well. Plates were then place in the incubator at 37° C. until treatment. Next day, compounds in appropriate concentrations were added to the wells and the plates incubated for additional 6 or 24 hours. DMSO was used as control (final concentration 0.5%).

Following the treatment with the compounds, cells were washed on the plates, lysed with the lysis buffer (2% SDS, 50 mM Tris, pH 8.0, 100 U/ml Pierce Universal Nuclease for Cell Lysis, complete EDTA-free Protease Inhibitor Cocktail) and incubated on ice for 45 min. The amount of protein was determined via BCA assay, and the equal quantity of each sample was loaded on the precast gel for the protein separation. Following the electrophoresis and transfer to the membrane, the membrane was stained with Ponceau (0.1% (w/v) Ponceau in 5% acetic acid). After total protein detection, the stain was washed out using TBST. After the antibody staining for MCL-1, PARP, cleaved Caspase 3 and α-tubulin, the membranes were washed and signals developed.

Densitometric values for MCL-1 were normalized to total protein level (the Ponceau Staining). [%] of protein reduction is calculated in comparison to the cells treated with DMSO, which stand for 0%.

The results for the 6 h treatment with 1 μM compounds are shown in Table 10, using the following labels:
<50% for 0-49% of MCL-1 protein reduction,
≥50% for 50-74% of MCL-1 protein reduction,
≥75% for 75-100% of MCL-1 protein reduction.

As illustrated in Table 10, the compounds of the present invention have the capability to induce degradation of MCL-1 protein in DMS 114 cell line.

TABLE 10
MCL-1 protein degradation results
for the compounds of the invention

| | Western blot, |
|---|---|
| Compound | % of protein reduction |
| 204 | ≥50 |
| 220 | ≥75 |
| 268 | ≥75 |
| 346 | ≥75 |

### Example 193: Dose-Dependent MCL-1 Protein Degradation and Induction of Apoptosis in Cancer Cells, Western Blot

The experiments were performed as described for the “OPM-2 cells: MCL-1 protein degradation, Western blot” and “DMS 114 cells: MCL-1 protein degradation, Western blot”.

In addition to MCL-1 and α-Tubulin, PARP and cleaved caspase-3 were detected to monitor apoptosis.

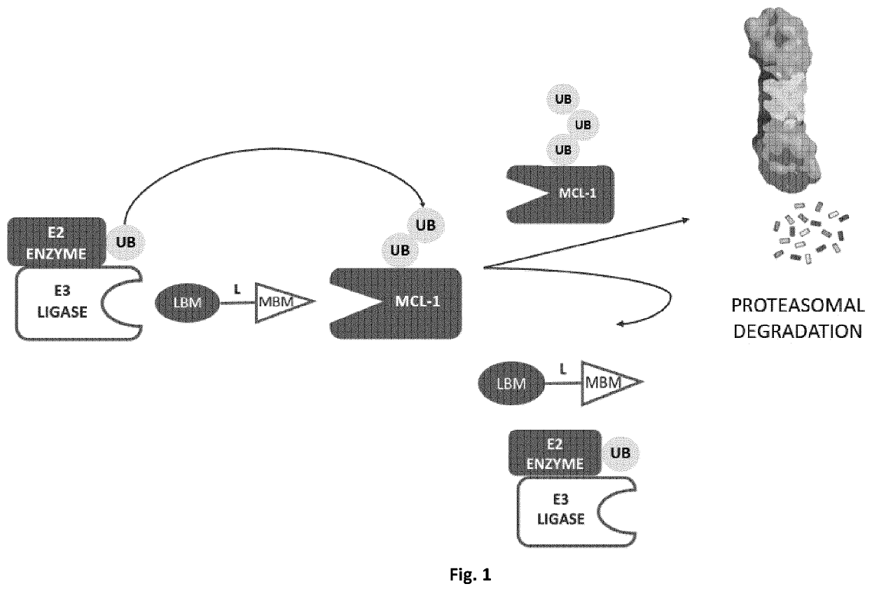

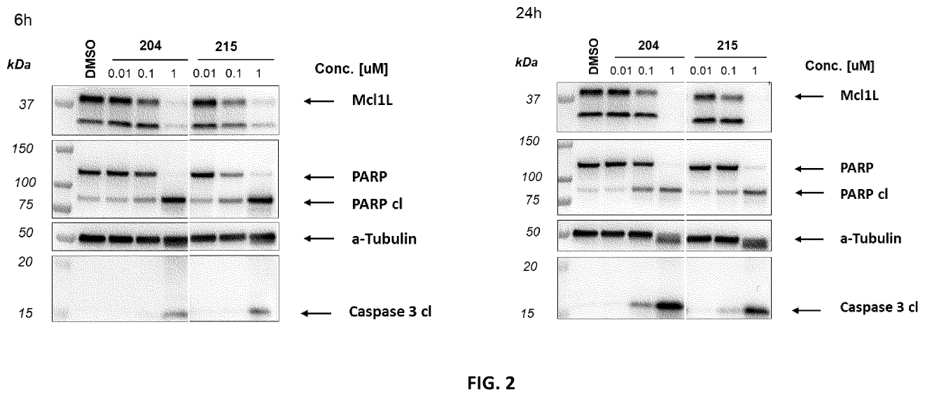

FIG. 2 presents the results of MCL-1 protein degradation and induction of apoptosis for the OPM-2 cells treated with 204 or 215 at 0.01, 0.1 or 1 μM concentration for 6 hours or 24 hours, as indicated.

All tested compounds decreased MCL-1 protein level in a dose-dependent manner. At 1 M concentration, all compounds degraded over 80% of Mcl1-L protein both at 6 h and 24 h-time point. All compounds induced apoptosis, observed by the appearance of cleaved caspase 3 and cleaved PARP, at 1 uM concentrations within 6 hours of treatment and 100 nM and 1 uM concentrations within 24 hours of treatment.

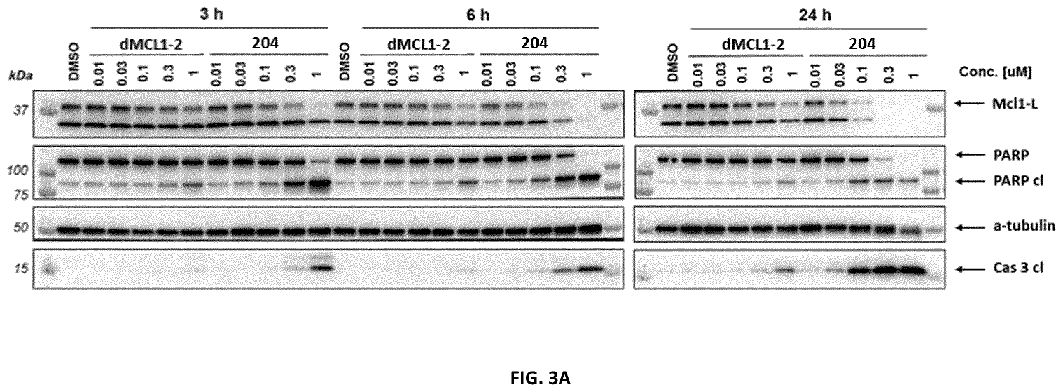

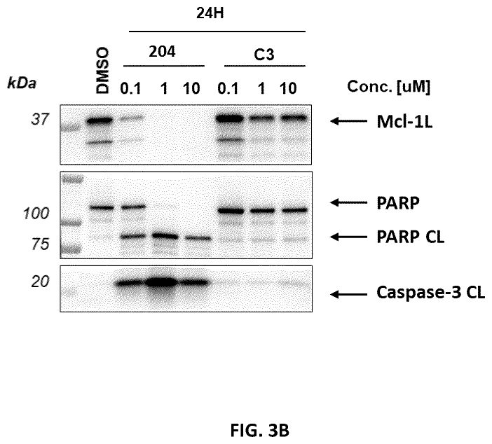

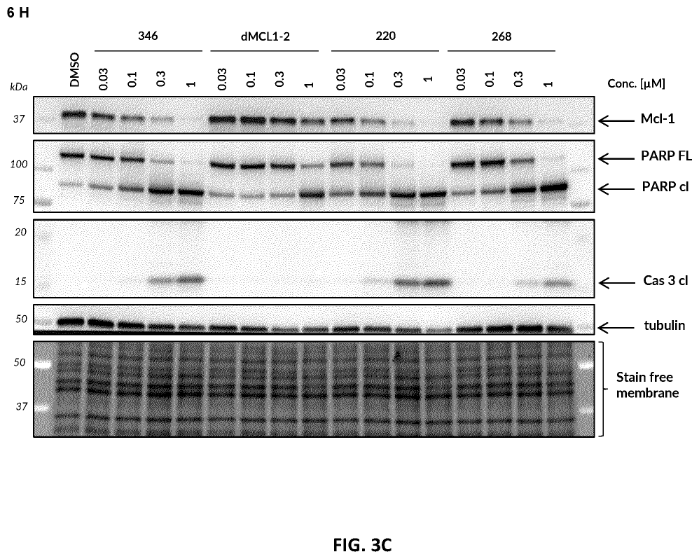

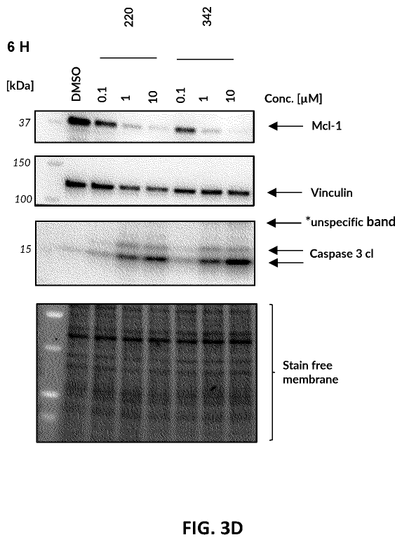

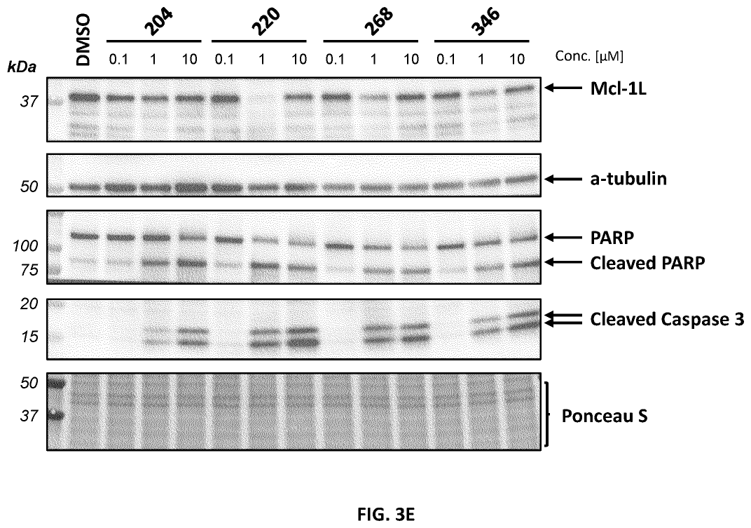

FIG. 3A presents the results of MCL-1 protein degradation and induction of apoptosis for the OPM-2 cells treated with 204 and control compounds dMCL1-2 (Papatzimas et al., J. Med. Chem. 2019, 62, 11, 5522-5540) at 0.01, 0.03, 0.1, 0.3 or 1 μM concentration for 3 hours, 6 hours or 24 hours, as indicated.

FIG. 3B presents the results of MCL-1 protein degradation and induction of apoptosis for the MV-4-11 cells treated with 204 and control compounds C3 (Wang Z et al., J. Med. Chem. 2019, 62, 17, 8152-8163) at 0.1, 1 or 10 μM concentration for 24 hours, as indicated.

The results show that the compounds of the invention, as exemplified by 204 are more potent in terms of MCL-1 protein degradation and induction of apoptosis.

FIG. 3C presents the results of MCL-1 protein degradation and induction of apoptosis for the OPM-2 cells treated with 220, 268, 346 and control compounds dMCL1-2 at 0.03, 0.1, 0.3, 1 μM concentration for 6 hours, as indicated.

FIG. 3D presents the results of MCL-1 protein degradation and induction of apoptosis for the OPM-2 cells treated with 220, 342 at 0.1, 1, 10 μM concentration for 6 hours, as indicated.

FIG. 3E presents the results of MCL-1 protein degradation and induction of apoptosis for the DMS 114 cells treated with 204, 220, 268, 346 at 0.1, 1, 10 μM concentration for 6 hours, as indicated.

As illustrated in FIG. 2 and FIG. 3, the compounds of the present invention, as exemplified by 204, 215, 220, 268 and 346 have the capability to induce degradation of MCL-1 protein and apoptosis of OPM2 cells with improved potency as compared to the known compound dMCL1-2 and C3. Moreover, the compounds of the present invention have the capability to induce degradation of MCL-1 protein and apoptosis of DMS 114 cells.

### Example 194: Cell Viability Assays

### OPM-2, MV-4-11, ARH-77 Cells:

The effect of various compounds of the invention on the viability of OPM-2, MV-4-11 and ARH-77 cell lines was investigated, using the CTG protocol described below.

OPM-2 and MV-4-11 cell lines are derived from multiple myeloma and acute myeloid leukaemia, respectively. Both cell lines are described in the literature as MCL-1 dependent lines, as opposed to lymphoblast-like ARH-77 cell line, which is described as independent of MCL-1 (Tron A E et al. Nat Commun. 2018; 9: 5341; Caenepeel S et al. Cancer Discov. 2018 December; 8(12):1582-1597).

Cells (OPM-2, MV-4-11 or ARH-77) were seeded on 348-well plates in a growth medium with FBS reduced to 1%. Next, compound stocks were pre-diluted in DMSO and added directly to the cells growth medium (final DMSO concentration: 0.5%). Compound concentrations used in this assay: 1/3 dilutions starting at 30 uM (9 to 12 points).

After 24 hours of incubation, cell viability was evaluated using CellTiter-Glo Luminescent Cell Viability Assay (Promega, cat #G7572). Luminescence signal was measured using a CLARIOstar Plus Multi-Mode Microplate Reader (574-590 nm).

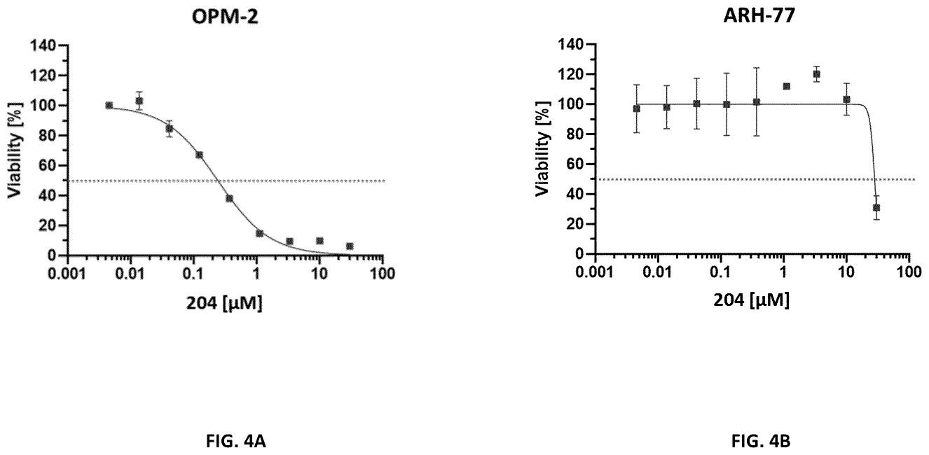

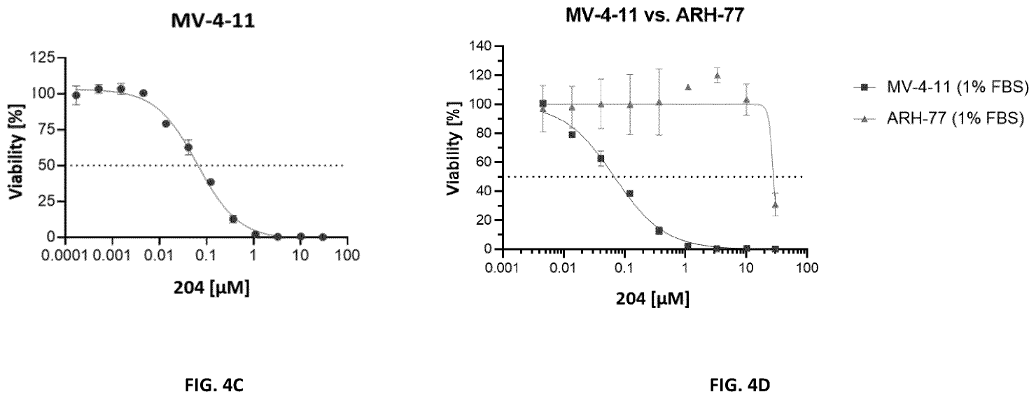

Results were normalized to DMSO control. Collected data were analyzed using GraphPad Prism to calculate pIC50 values, which are presented in Table 11. The representative dose-response graphs for 204 are shown in FIG. 4.

### DMS 114 Cells:

The effect of various compounds of the invention on the viability of DMS 114 cell line was investigated, using the CTG protocol described below.

DMS 114 cell line is derived from small-cell lung cancer, which is described in the literature as MCL-1 dependent lines [Yasuda Y et al. Cell Death & Disease 2020; 11: 177].

DMS 114 cells were detached with trypsin and then resuspended in growth medium containing 10% FBS and counted. Cell suspension was then adjusted to 5×10⁴ cells/ml using growth medium containing 10% FBS. Afterwards, 3000 cells/well were seeded on 384-well plates (50 μl). Plates were then placed in the incubator overnight until treated. Next day, compounds were added using ECHO 555 liquid handler. Compound concentrations used in this assay: 1/3 dilutions starting at 30 uM (9 to 12 points).

After 72 hours of incubation, cell viability was evaluated using CellTiter-Glo Luminescent Cell Viability Assay (Promega, cat #G7572). Luminescence signal was measured using a CLARIOstar Plus Multi-Mode Microplate Reader (574-590 nm).

TABLE 11
The effect of various compounds of the invention on the viability
of OPM-2, MV-4-11, DMS 114 and ARH-77 cell lines

| | Average | Average | Average | Average |
|---|---|---|---|---|
| | pIC50 by | pIC50 by | pIC50 by | pIC50 by |
| | molecule | molecule | molecule | molecule |
| Compound | OPM-2 | MV-4-11 | DMS 114 | ARH-77 |
| 201 | ≥6.0 | ≥7.0 | | |
| 203 | | ≥6.0 | | |
| 204 | ≥6.0 | ≥7.0 | | |
| 205 | | ≥6.5 | | |
| 206 | ≥6.5 | ≥7.0 | | |
| 207 | ≥6.0 | ≥6.5 | | <5.0 |
| 210 | ≥6.0 | ≥7.0 | | <5.0 |
| 211 | ≥5.5 | ≥6.5 | | <5.0 |
| 213 | | ≥6.5 | | <5.0 |
| 214 | ≥6.5 | ≥7.0 | | |
| 215 | ≥6.0 | ≥7.0 | | |
| 216 | ≥7.0 | ≥7.0 | ≥5.5 | <5.0 |
| 217 | | ≥6.0 | | |
| 218 | | ≥6.0 | | <5.0 |
| 220 | | ≥7.5 | ≥6.5 | <5.0 |
| 221 | | ≥5.0 | | <4.5 |
| 222 | | ≥6.5 | | <5.0 |
| 223 | | ≥6.5 | | <5.0 |
| 224 | | ≥6.5 | | <5.0 |
| 225 | | ≥6.5 | | <5.0 |
| 226 | | ≥7.0 | | <5.0 |
| 241 | | ≥6.0 | | |
| 249 | | ≥6.0 | | |
| 263 | ≥6.0 | ≥6.5 | | |
| 265 | | ≥6.0 | | |
| 266 | ≥6.5 | ≥7.0 | | |
| 292 | | ≥7.0 | | <5.0 |
| 294 | | ≥7.5 | ≥6.0 | <5.0 |
| 295 | | ≥7.5 | | <5.0 |
| 296 | | ≥7.5 | | <5.0 |
| 297 | | ≥7.0 | | <5.0 |
| 298 | | ≥7.0 | ≥6.0 | <5.0 |
| 299 | | ≥7.0 | | <5.0 |
| 300 | | ≥7.0 | | <5.5 |
| 301 | | ≥7.0 | | <5.0 |
| 306 | | ≥7.0 | | <5.0 |
| 308 | | ≥6.5 | | <5.5 |
| 315 | | ≥7.5 | | <5.5 |
| 316 | | ≥7.0 | | <5.5 |
| 317 | | ≥7.5 | | <5.5 |
| 318 | | ≥7.5 | | <5.0 |
| 319 | | ≥7.5 | ≥5.5 | <5.0 |
| 320 | | ≥7.0 | | <5.0 |
| 321 | | ≥6.5 | | <5.0 |
| 325 | | ≥7.0 | | <5.0 |
| 326 | | ≥6.5 | | <5.0 |
| 327 | | ≥7.0 | | <5.0 |
| 328 | | ≥7.5 | | <5.5 |
| 329 | | ≥6.5 | | <5.0 |
| 330 | | ≥7.0 | | <5.0 |
| 331 | | ≥6.5 | | <5.0 |
| 332 | | ≥6.0 | | <4.5 |
| 333 | | ≥6.0 | | |
| 334 | | ≥7.0 | | <5.5 |
| 335 | | ≥7.5 | | <5.5 |
| 336 | | ≥7.0 | | <5.0 |
| 337 | | ≥6.5 | | <5.0 |
| 338 | | ≥7.5 | ≥5.0 | <5.0 |
| 342 | | ≥7.5 | ≥6.0 | <5.0 |
| 345 | | ≥7.0 | | <5.0 |
| 269 | | ≥7.0 | ≥5.5 | <5.0 |
| 270 | | ≥7.0 | | <5.0 |
| 271 | | ≥7.0 | | <5.0 |
| 272 | | ≥7.0 | | <5.0 |
| 273 | | ≥6.5 | | <5.0 |
| 274 | | ≥7.0 | | <5.0 |
| 275 | | ≥7.0 | ≥5.5 | <5.0 |
| 276 | | ≥7.0 | | <5.0 |
| 277 | | ≥7.0 | ≥5.5 | <5.0 |
| 278 | | ≥7.0 | ≥5.5 | <5.0 |
| 280 | | ≥7.0 | | <5.5 |
| 281 | | ≥7.0 | | <5.0 |
| 282 | | ≥7.5 | | <5.5 |
| 284 | | ≥7.0 | | <5.5 |
| 285 | | ≥7.0 | | <5.0 |
| 287 | | ≥6.0 | | <5.5 |
| 346 | | ≥7.0 | ≥5.5 | <5.0 |
| 348 | | ≥6.5 | | <5.0 |
| 349 | | ≥7.5 | ≥6.0 | <5.0 |
| 351 | | ≥6.5 | | <4.5 |
| 354 | | ≥7.0 | | <5.0 |
| C3 | | 5.0 | | |

As illustrated in Table 11 and FIG. 4, the compounds of the present invention have the capability to decrease the viability of multiple myeloma and acute myeloid leukaemia cells, as well as small cell lung cancer cell line, thus can be useful in the treatment of cancer. Simultaneously, the compounds of the present invention have no/little impact on the viability of ARH-77, which further supports a specific, MCL-1 dependent activity of said compounds.

### Example 195: Assessment of Cardiac Safety; Caspase 3 and 7 Activation

The effect of various compounds of the invention and a reference compound AZD5991 on cardiac safety was investigated by the evaluation of caspase 3 and 7 activation using Caspase-Glo 3/7 Assay (Promega; G8092) in iCELL™ Cardiomyocytes from FUJIFILM Cellular Dynamics (Catalog number: 11713). In the Caspase-Glo® 3/7 Assay, the luminescence (measured as Relative Luciferase Units, RLU) is proportional to the amount of caspase activity present in each sample.

The assay is initiated by thawing and seeding iCell® Cardiomyocytes as specified in the User Guide. After 48 hours, the cells are washed to remove unattached cells and debris and immediately treated with compounds or DMSO diluted in the maintenance medium (FCDI; cat: M1003). Compound concentrations used in this assay: 7 points 1/3 dilution curve starting at 100 uM. After 24 hours of incubation with compounds, caspase activity is measured by the addition of the Caspase-Glo® 3/7 Reagent followed by the luminescence measurement.

The analysis of the results is performed by calculation of the fold change in caspase 3/7 activity at a given compound concentration. Fold change is calculated with the formula:

$$FOLD CHANGE = [ \text{} ( RLU Xi ) - ( RLU BG ) ] / \{ Average [ ( RLU C ) - ( RLU BG ) ] \}$$

where:
RLU Xi is the Relative Luciferase Units of sample I;
RLU BG is the Background Relative Luciferase Units;
RLU C is the Relative Luciferase Units of Control (DMSO).

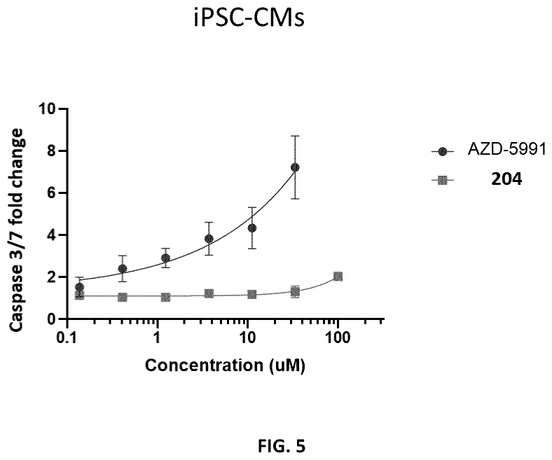

The experiments were performed in technical duplicates. The results are presented in Table 12. A representative dose-response curve is presented in FIG. 5.

TABLE 12
The effect of various compounds of the invention and the reference
compound on the caspase 3/7 activity in cardiomyocytes

| Compound | Fold change at 1.2 uM |
|---|---|
| 204 | ≤1.5 ± 0.2 |
| 206 | ≤1.5 ± 0.2 |
| 266 | ≤1.5 ± 0.2 |
| 214 | ≤1.5 ± 0.2 |
| AZD5991 | 2.9 ± 0.5 |

As illustrated in Table 12 and FIG. 5, the compounds of the present invention, as exemplified by 204, 206, 266, 214 show little to no activity in caspase activity assay in cardiomyocytes, as opposed to the reference compound AZD5991.

Together, the results described in Examples 192, 193 and 194 demonstrate a surprising technical effect of compounds of the present invention which is the ability to induce apoptosis in cancer cells combined with no/little activity in cardiomyocytes.

### Example 196: Assessment of Cardiac Safety: hiPSC-CM Cell Viability Assays

The effect of various compounds of the invention and a reference compounds AZD5991 and MIK665 on cardiac safety was investigated by the evaluation of viability with CTG assay (Promega; cat #G7572) in iCELL™ Cardiomyocytes from FUJIFILM Cellular Dynamics (Catalog number: 11713).

Cells were thawed, counted using an automated cell counter (TC20, Bio-Rad) and seeded in 96 well plates (Greiner-BioOne; 655098) previously coated with 0.1% gelatin (Sigma-Aldrich; G1890) using Plating Medium (#M1001) following the instructions of the iCELL cardiomyocytes User Guide. Forty ml of Plating Medium was used to equally distribute the cells in four 96 well plates. Cells were then placed in the incubator at 37° C., 5% CO₂ and left undisturbed for 48 hours. Compounds were serially diluted in DMSO and then further diluted to final assay concentration using maintenance medium (#M1003). Final DMSO concentration was 0.5%. Forty eight hours after plating, non-adherent cells and debris were washed as described in the User Guide. Plating medium was then replaced with maintenance medium containing diluted compounds or DMSO (as control). Medium was then changed every 2 days with fresh medium containing compounds or DMSO until the end of the treatment. On day 14, only medium without compounds was used immediately before the CTG assay. Luminescence signal was measured using a CLARIOstar Plus Multi-Mode Microplate Reader (574-590 nm).

TABLE 13
The effect of various compounds of the invention and
the reference compounds on the viability of hiPSC-CM

| Compound | Average pIC50 by molecule hiPSC-CM |
|---|---|
| 346 | 5.0 |
| 268 | <4.4 |
| 220 | 5.0 |
| AZD5991 | 6.2 |
| MIK665 | 6.9 |

As illustrated in Table 13, the compounds of the present invention, as exemplified by 220, 268, 346 show much lower activity in viability assay in cardiomyocytes, as opposed to the reference compounds MIK665 and AZD5991.

### Example 197: Induction of Apoptosis in Cancer Cells, Flow Cytometry

The effect of compound 204 of the invention MV-4-11 cells was investigated using the following protocol.

MV-4-11 cells were seeded on 24-well plates (1 ml) in growth medium containing 1% FBS. Immediately after seeding, compounds in appropriate concentrations were added directly to the cell culture medium. After 24 hours incubation, cells were washed twice with cold DPBS and the staining was performed using the FITC Annexin V Apoptosis Detection Kit I (BD Pharmingen; 556547). Samples were then analyzed by flow cytometry (LSR Fortessa X-20; BD Biosciences) within 1 hr. Results were evaluated using FlowJo (V10) software.

FIG. 6 presents the induction of apoptosis in MV-4-11 cells treated with 204 at 1, 3, 10, 30, and 100 nM concentration for 24 hours, as indicated.

As illustrated in FIG. 6, the compounds of the invention, as exemplified by 204, cause a concentration dependent increase of the population of late apoptotic/cell death (Annexin+/PI+) cells after 24 hours of treatment.

### Example 198: Monitoring Mitochondrial Potential Changes in Cancer Cells Using Flow Cytometry

The effect of compounds 220 and 268 of the invention in MV-4-11 cells was investigated using the following protocol.

MV-4-11 cells were seeded on 384-well plates (0.05 ml) in growth medium containing 1% FBS and 50 nM tetramethylrhodamine ethyl ester (TMRE). Immediately after seeding, compounds in appropriate concentrations were added directly to the cell culture medium. After 5 hours incubation, cells were analyzed by flow cytometry (LSR Fortessa X-20; BD Biosciences). Results were evaluated using FlowJo (V10) software.

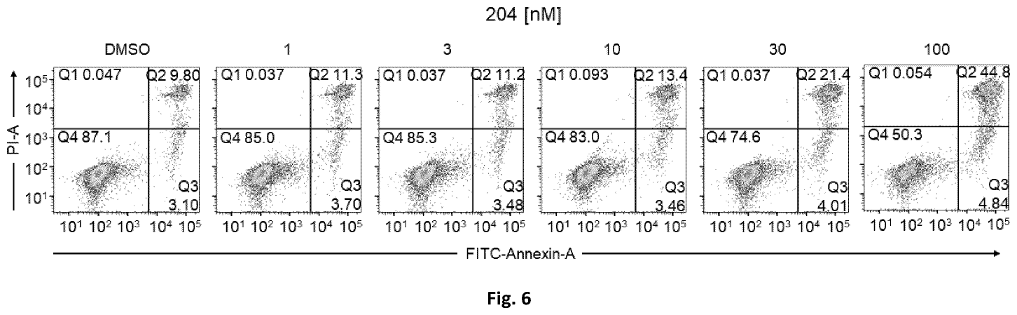

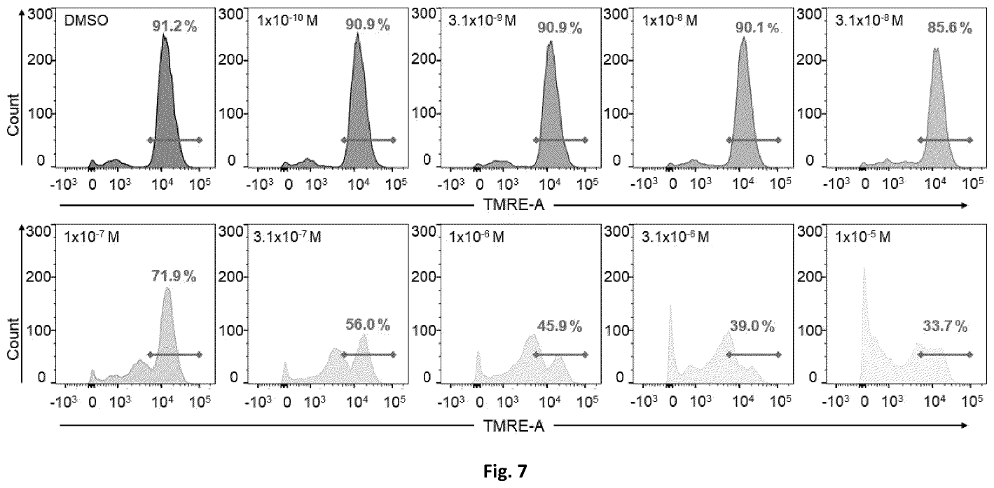

FIG. 7 presents the effect of the compound in the mitochondrial potential of MV-4-11 cells treated with 220 for 5 hours. Compound concentration is specified in the top-left corner of each histogram; Dimethyl sulfoxide (DMSO) was used as vehicle control. Population of interest and the percentage of cells included are highlighted with a red bar and shown in bold red ink, respectively.

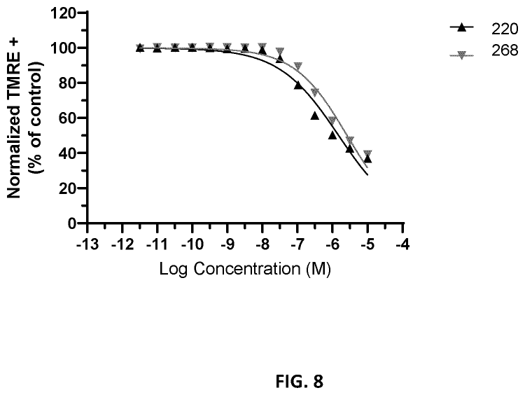

FIG. 8 presents the concentration dependent decrease (compared to DMSO control) of the population of cells with normal polarized mitochondria after treatment of MV-4-11 cells with the compounds 220 and 268 for 5 hours.

As illustrated in FIGS. 7 and 8, the compounds of the invention, as exemplified by 220 and 268, cause a concentration dependent depolarization of mitochondria clearly observed by the decrease in counted events of population of interest (peak at 1.5×10⁴ a.u.) indicative of the onset of apoptosis/cell death.

### ABBREVIATIONS AND DEFINITIONS

A list of the abbreviations used in the present application is shown in Table 14, below:

TABLE 14
Abbreviations

| Abbreviation | Meaning |
|---|---|
| CRBN | Cereblon |
| DDB1 | damaged DNA binding protein 1 |
| TFA | trifluoroacetic acid |
| CDI | carbonyldiimidazole |
| FA | formic acid |
| MsCl | methanesulfonyl chloride |
| DBAD | di-tert-butyl azodicarboxylate |
| Tris | Tris(hydroxymethyl)aminomethane |
| DMSO | Dimethylsulfoxide |
| THF | tetrahydrofuran |
| DMF | dimethylformamide |
| DMA | dimethylacetamide |
| DMAP | 4-dimethylaminopyridine |
| EDC | 1-ethyl-3-(3-dimethylaminopropyl)carbodiimide |
| HPLC | High performance liquid chromatography |
| NCS | N-chlorosuccinimide |
| DIPEA | N,N-diisopropylethylamine |
| MTBE | Methyl tert-butyl ether |
| HATU | Hexafluorophosphate Azabenzotriazole Tetramethyl |
| | Uronium (1-[Bis(dimethylamino)methylene]-1H-1,2,3- |
| | triazol[4,5-b]pyridinium-2-oxide hexafluorophosphate) |
| m-CPBA | meta-chloroperbenzoic acid |
| Pd(amphos)Cl₂ | Bis(di-tert-butyl(4- |
| | dimethylaminophenyl)phosphine)dichloropalladium(II) |
| Pd(dppf)Cl₂ | [1,1′- |
| CH₂Cl₂ | bis(diphenylphosphino)ferrocene]dichloropalladium(II), |
| | complex with dichloromethane |
| DTT | dithiothreitol |
| RT | Room temperature |

As used herein, the term “room temperature” means a temperature of between 20° C. and 25° C.

1. A compound of formula (I)
[MCL-1 ligand moiety]-linker-[ligase ligand moiety] (I)
or a salt, solvate, hydrate, isomer or prodrug thereof,
wherein [ligase ligand moiety] is: 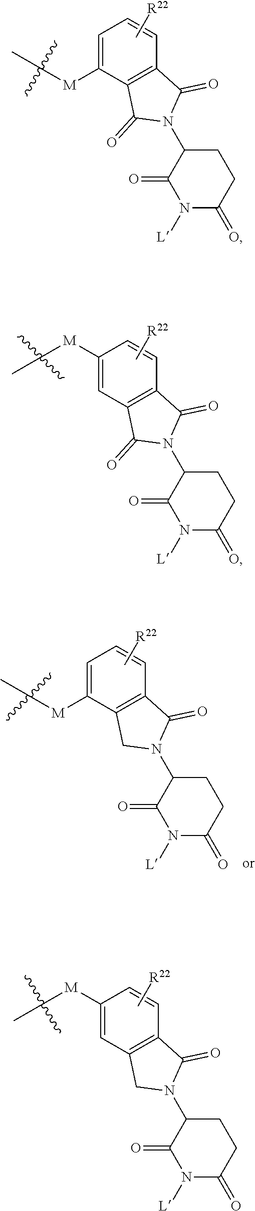
wherein
M is O, S or NH, or is absent; [symbol]
indicates attachment to R¹⁸ of the linker;
R²² is hydrogen, halogen or an amino group; and
L′ is hydrogen, alkyl, benzyl, acetyl or pivaloyl;
[MCL-1 ligand moiety] is a compound of Formula (A), Formula (B) or Formula (C)
wherein 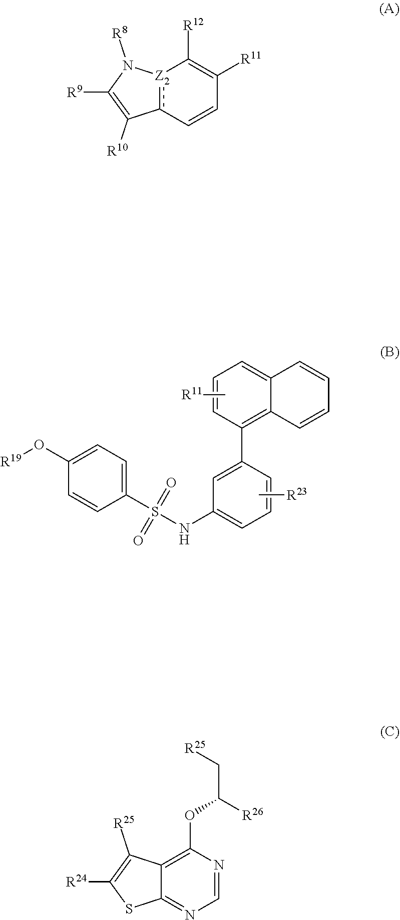 [symbol]
is a single bond or a double bond;
R⁸ is H, R¹⁹, or C₁-C₆ alkyl optionally substituted with morpholine;
R⁹ is —C(O)OH, —C(O)OC₁-C₆alkyl; —C(O)NH₂; —C(O)OR¹⁹ or —C(O)NHR¹⁹,
R¹⁰ is —C₂₋₅alkyl-O—R¹³ or —C₂₋₅alkyl-NMe-R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹¹ is H, halogen or C₁-C₆ alkyl,
R¹² is H, 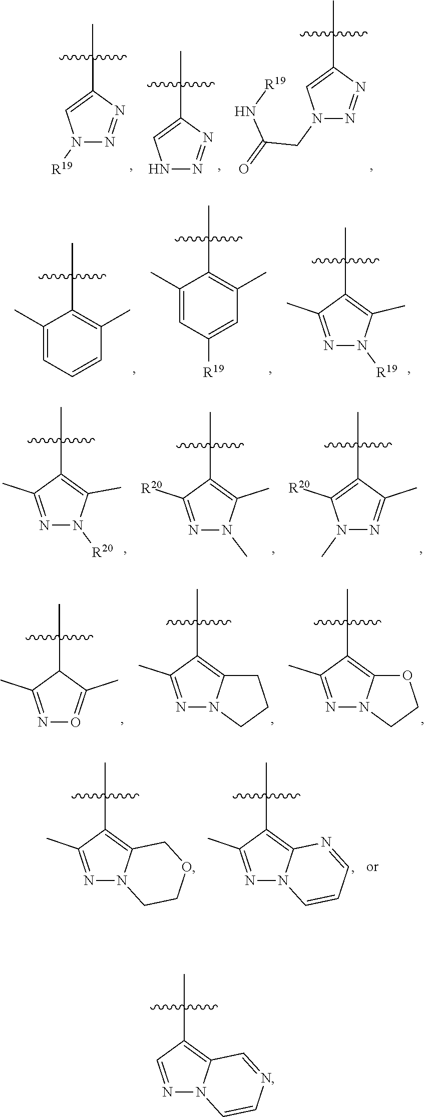
wherein R²⁰ is Me, —CH₂—OMe, —CH₂—O-bromobenzaldehyde, or 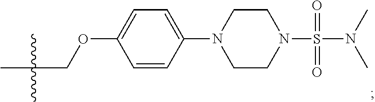 [SMILES: *COc1ccc(N2CCN(S(=O)(=O)N(C)C)CC2)cc1]
or when R¹² is 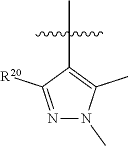
and R¹⁰ is —O-naphthyl substituted with —O— or —S—, then R²⁰ is 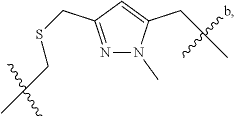
wherein [symbol] indicates attachment to —O— or —S— of R¹⁰;
and wherein
R¹⁹ is a bond connected to R¹⁴ of the linker;
R²³ is —C(O)OH or —C(O)OC₁-C₆alkyl;
Z₂ is N or C, wherein when Z₂ is N, then [symbol] is a single bond; and when Z₂ is C, then [symbol] is a double bond,
R²⁴ is furan optionally substituted with at least one halogen,
each R²⁵ is independently phenyl substituted with —OR²⁸ and optionally further substituted with at least one substituent selected from halogen and C₁-C₆ alkyl;
R²⁶ is —C(O)OR¹⁹ or —C(O)NHR¹⁹; and
each R²⁸ is independently —C₁₋₃alkyl-(N-alkyl piperazine) or —C₁₋₃alkyl-(N-haloalkylpyrazole)
and wherein each of Formula (A), Formula (B) and Formula (C) contains a single R¹⁹;
and wherein [linker] has the following formula
R¹⁴—R¹⁵—R¹⁶—R¹⁷—R¹⁸
wherein
R¹⁴ is —C₁₋₆alkyl, —C₂₋₆alkenyl, —C₂₋₆alkynyl, C₁₋₆ alkyl-N(C₁₋₆ alkyl)-, —C(O)—, —SO₂— or is absent
R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, (C₂H₄—O)ₓ, (C₃H₆—O)ₓ, or is absent
x is 1-10
y is 2-10
R¹⁸ is —C₁₋₆ alkyl, heterocycloalkyl, or is absent
wherein at least one of R¹⁴-R¹⁸ is present
with the proviso that:
when
R¹⁰ is —C₃H₆—O-naphthyl,
R¹² is 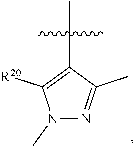
and
R²⁰ is 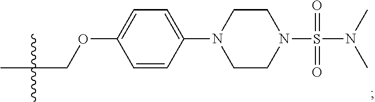 [SMILES: *COc1ccc(N2CCN(S(=O)(=O)N(C)C)CC2)cc1]
then R⁹ is —C(O)OH, —C(O)OC₁-C₆alkyl or —C(O)NH₂, and [ligase ligand moiety]

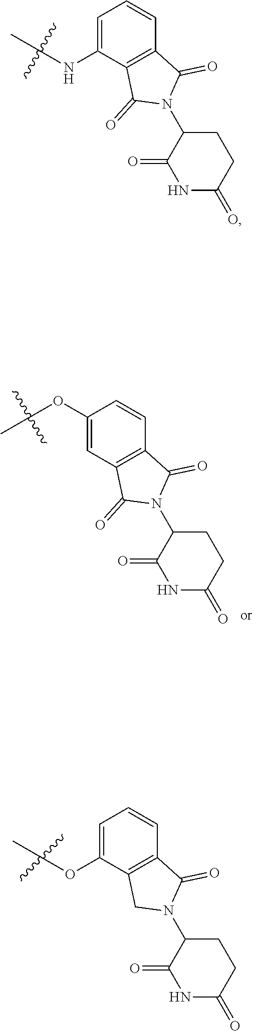 [SMILES: *Nc1cccc2c1C(=O)N(C1CCC(=O)NC1=O)C2=O ; *Oc1ccc2c(c1)C(=O)N(C1CCC(=O)NC1=O)C2=O ; *Oc1cccc2c1CN(C1CCC(=O)NC1=O)C2=O]

2. The compound of claim 1, wherein R²² is hydrogen or an amino group.

3. The compound of claim 2, wherein R²² is hydrogen.

4. The compound of any preceding claim, wherein L′ is hydrogen or methyl.

5. The compound of claim 4, wherein L′ is hydrogen.

6. The compound of any preceding claim, wherein M is O or NH, or is absent.

7. The compound of any preceding claim, wherein [ligase ligand moiety] is:

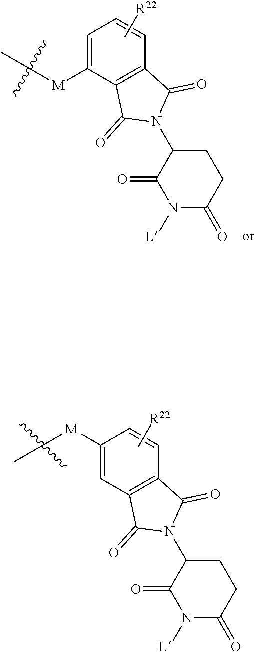

8. The compound of claim 7, wherein [ligase ligand moiety] is:

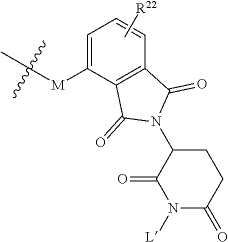

9. The compound of claim 7, wherein [ligase ligand moiety] is:

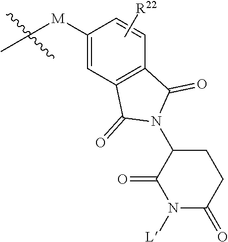

10. The compound of claim 7, wherein [ligase ligand moiety] is

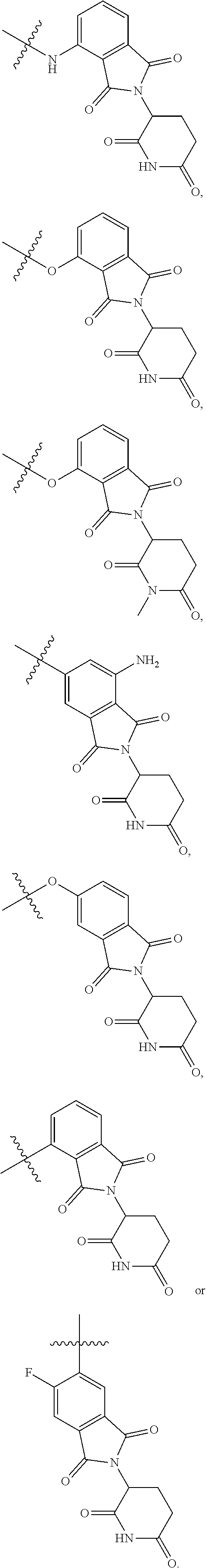

11. The compound of any one of claims 1-6, wherein [ligase ligand moiety] is:

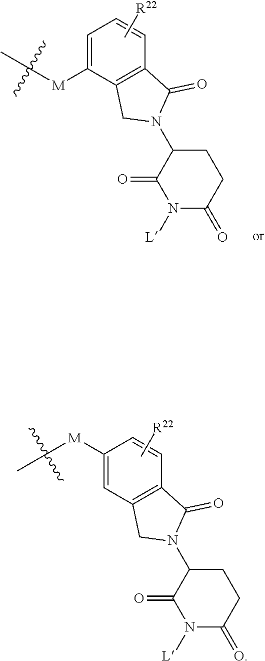

12. The compound of claim 11, wherein [ligase ligand moiety] is:

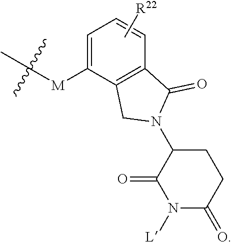

13. The compound of claim 11, wherein [ligase ligand moiety] is:

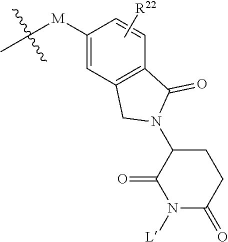

14. The compound of claim 11, wherein [ligase ligand moiety] is

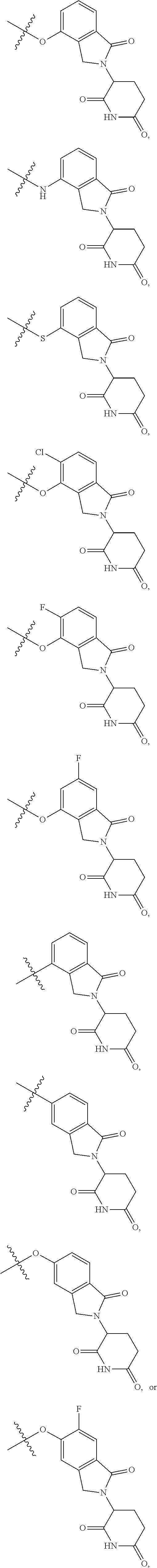

15. The compound of any preceding claim, wherein R¹⁴ is —C₁₋₆alkyl, —C₂₋₆alkenyl, —C₂₋₆alkynyl, —C(O)—, —SO₂— or is absent.

16. The compound of any preceding claim, wherein R¹⁵ is cycloalkyl, heterocycloalkyl, aryl, heteroaryl, —C₁₋₆ alkyl-NH—, -cycloalkyl-NH— or is absent.

17. The compound of any one of claims 1-14, wherein
R¹⁴ is —C₁₋₆ alkyl, —C₁₋₆ alkyl-N(Me)-, —SO₂— or is absent
R¹⁵ is piperazine, bridged piperazine, piperazine N-oxide, piperazine cation, —C₁₋₆ alkyl-NH—, —C₁₋₆ alkyl-N(Me)-, 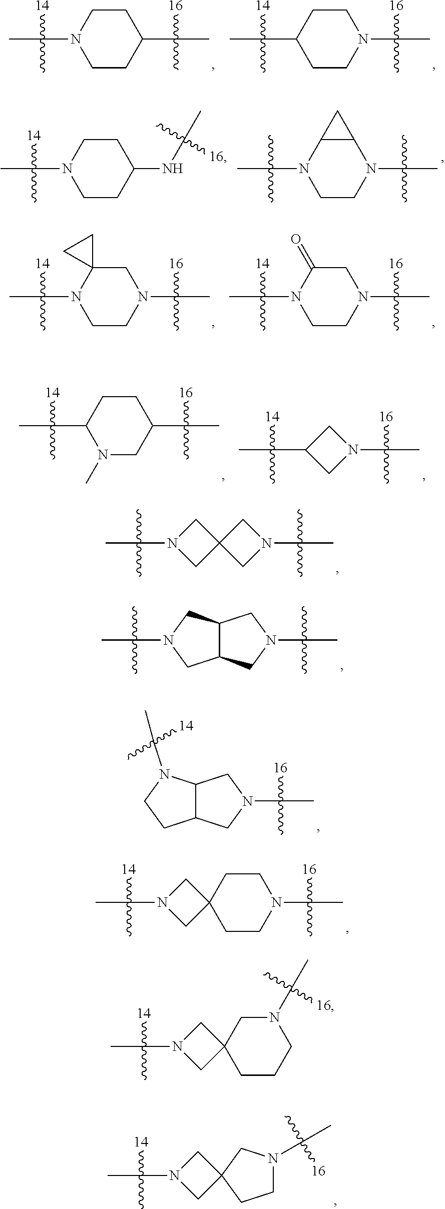
or is absent,
wherein [symbol] indicates attachment to R¹⁴ [symbol] and indicates attachment to R¹⁶,
R¹⁷ is —CH₂(C₂H₄—O)_{y}, (C₂H₄—O)ₓ, (C₃H₆—O)ₓ, or is absent
x is 1-6
y is 2-6
R¹⁸ is —C₁₋₆ alkyl, piperazine, 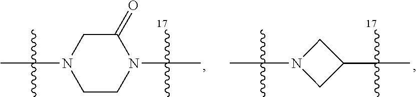 [SMILES: *N1CCN(*)C(=O)C1 ; *C1CN(*)C1]
or is absent,
wherein [symbol] indicates attachment to R¹⁷,
wherein at least one of R¹⁴-R¹⁸ is present.

18. The compound of any preceding claim, wherein
R¹⁴ is —C₁₋₆ alkyl, —SO₂— or is absent
R¹⁵ is piperazine, bridged piperazine, piperazine N-oxide, piperazine cation, —C₁₋₆ alkyl-NH—, 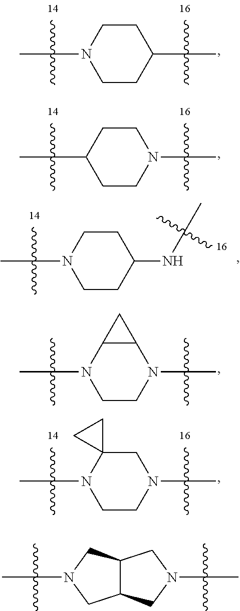
or is absent, wherein [symbol] indicates attachment to R¹⁴ and [symbol] indicates attachment to R¹⁶,
R¹⁶ is —C₁₋₆ alkyl, —C(O)—, —C(O)—NH—, —CH₂—C(O)—NH— or is absent
R¹⁷ is —CH₂(C₂H₄—O)_{y}, (C₂H₄—O)ₓ, (C₃H₆—O)ₓ, or is absent
x is 1-6
y is 2-6
R¹⁸ is —C₁₋₆ alkyl, piperazine, or is absent
wherein at least one of R¹⁴-R¹⁸ is present.

19. The compound of any preceding claim, wherein R¹⁸ is —C₁₋₆ alkyl or is absent.

20. The compound of any preceding claim, wherein when R¹⁴ is —SO₂—, at least two of R¹⁵-R¹⁸ are present, and at least one of R¹⁵—R¹⁸ is not C₁₋₆ alkyl.

21. The compound of any preceding claim, wherein
R¹⁴ is —SO₂—
R¹⁵ is —C₁₋₆ alkyl-NH—
R¹⁶ is —C(O)—
R¹⁷ is —CH₂(C₂H₄—O)_{y}, (C₂H₄—O)ₓ or is absent
R¹⁸ is —C₂₋₄ alkyl.

22. The compound of claim 22, wherein
R¹⁵ is —C₂alkyl-NH—
x is 1 or 2
y is 1.

23. The compound of any one of claims 1-19, wherein when R¹⁵ is piperazine, bridged piperazine, piperazine N-oxide, piperazine cation, —C₁₋₆ alkyl-NH—, 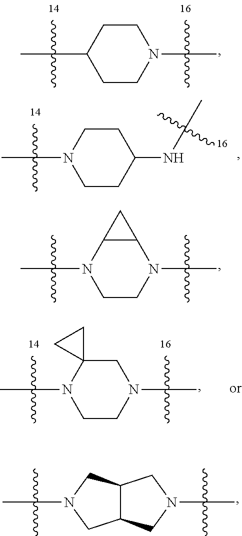
then R¹⁴ is —C₁₋₆ alkyl.

24. The compound of any one of claims 1-19, wherein
R¹⁴ is —C₁₋₆ alkyl,
R¹⁵ is piperazine, bridged piperazine, piperazine N-oxide, 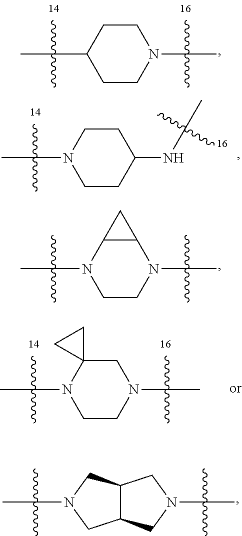
R¹⁶ is —C(O)—, —CH₂—C(O)—NH—, or is absent
R¹⁷ is —CH₂(C₂H₄—O)_{y}, (C₂H₄—O)ₓ, (C₃H₆—O)ₓ, or is absent
R¹⁸ is —C₁₋₆ alkyl.
wherein when R¹⁶ and R¹⁷ are absent, R¹⁸ is —C₃₋₆ alkyl.

25. The compound of claim 24, wherein
R¹⁴ is —C₂ alkyl,
x is 1, 2 or 6
y is 2.

26. The compound of any one of claims 1-19, wherein
R¹⁴ is absent
R¹⁵ is absent
R¹⁶ is —C(O)—NH—, or is absent
R¹⁷ is —CH₂(C₂H₄—O)_{y}, (C₂H₄—O)ₓ, (C₃H₆—O)ₓ, or is absent
R¹⁸ is —C₁₋₆ alkyl.

27. The compound of any preceding claim, wherein at least one of R¹⁴-R¹⁸ is not —C₁₋₆ alkyl.

28. The compound of claim 26 or 27, wherein
x is 1, 2 or 3
y is 2
R¹⁸ is —C₂₋₆ alkyl.

29. The compound of any preceding claim, wherein when R¹⁵ is —C₁₋₆ alkyl-NH—, at least one of R¹⁶—R¹⁸ is present.

30. The compound of any one of claims 1-28, wherein when R¹⁷ is —CH₂(C₂H₄—O)_{y}, (C₂H₄—O)ₓ or (C₃H₆—O)ₓ, at least one of R¹⁴—R¹⁶ and R¹⁸ is present, wherein at least one of R¹⁴ and R¹⁸ is not —C₁₋₆ alkyl.

31. The compound of any preceding claim, wherein [linker] is selected from 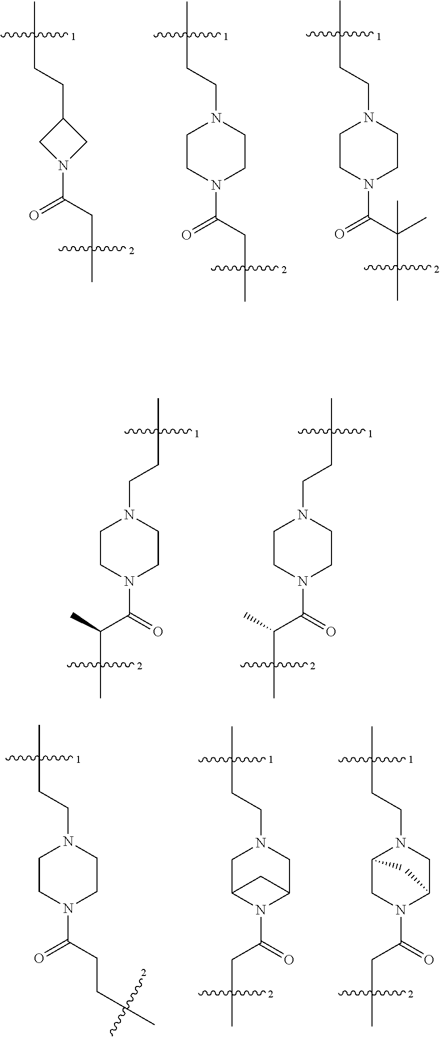 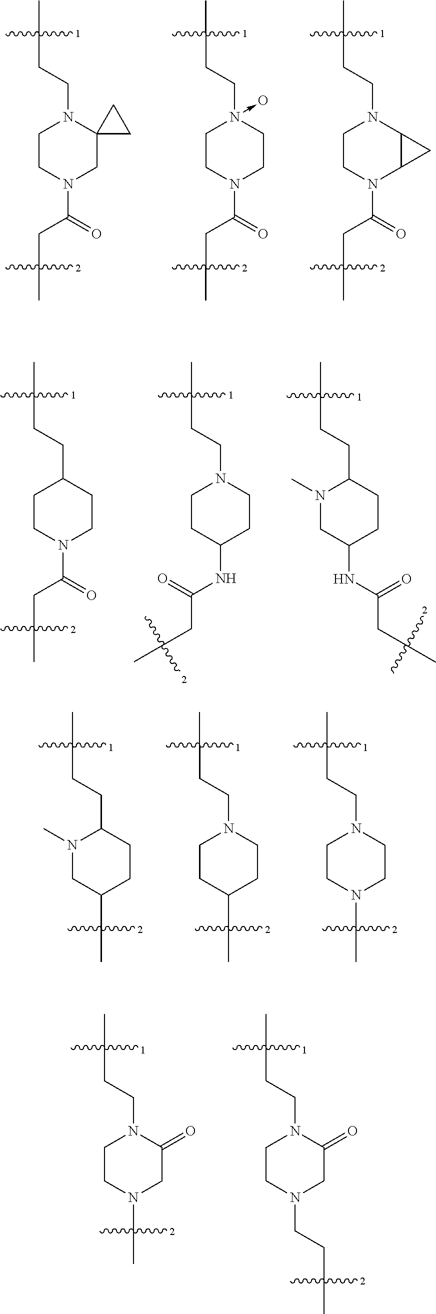 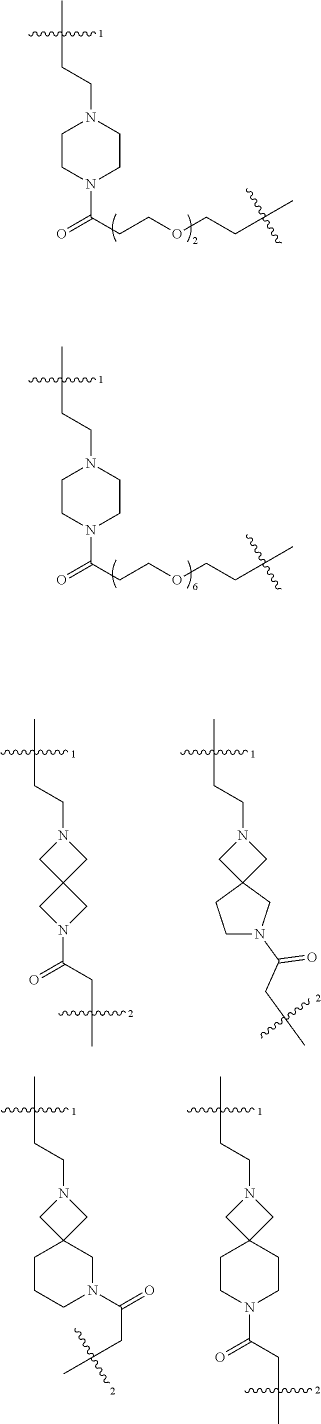 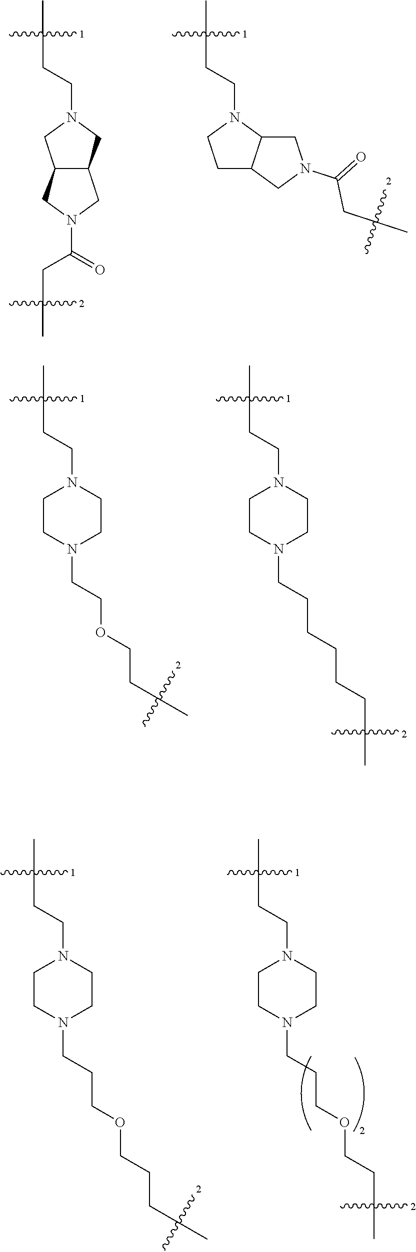 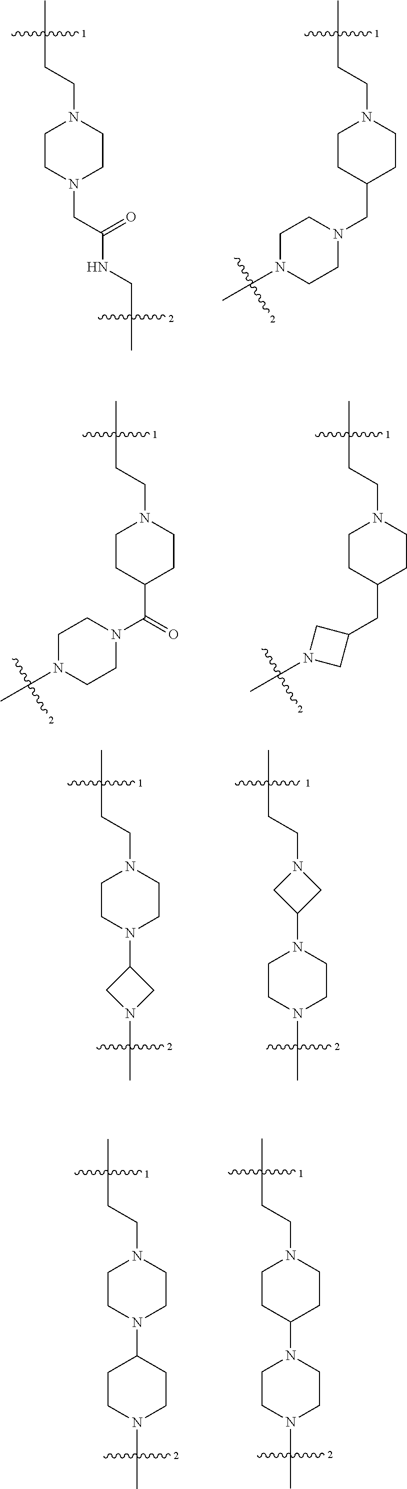 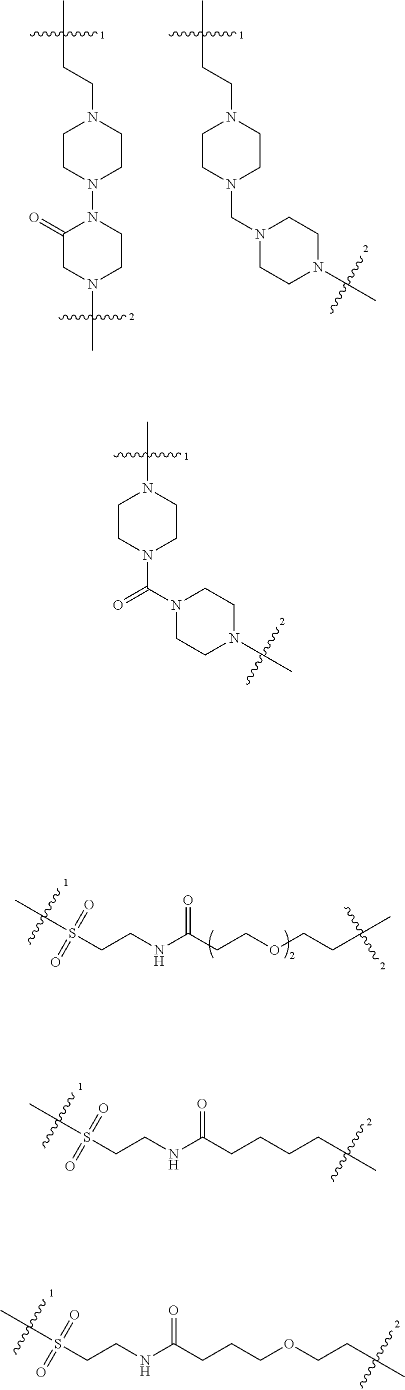 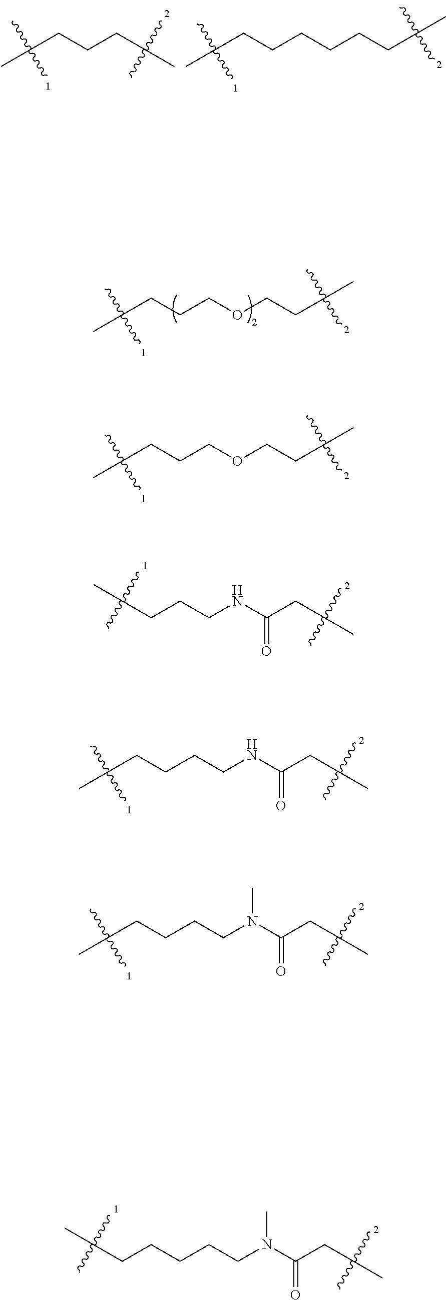 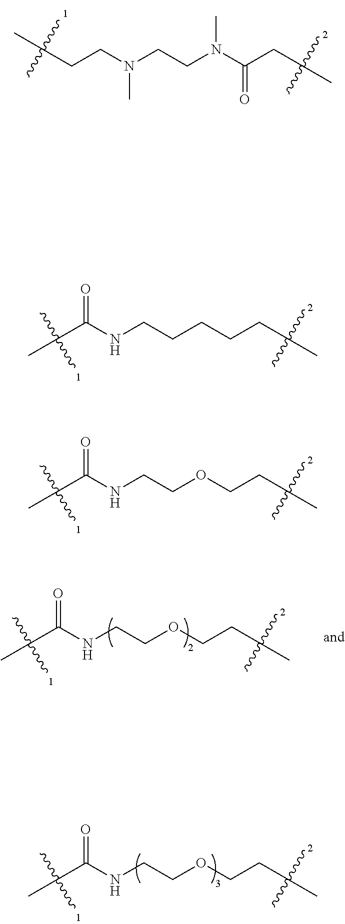
wherein [symbol]
indicates attachment to [MCL-1 ligand moiety] and [symbol]
indicates attachment to [ligase ligand moiety].

32. The compound of any preceding claim, wherein R¹⁰ is —C₂₋₅alkyl-O—R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the naphthyl is optionally substituted with —O— or —S—.

33. The compound of any preceding claim, wherein R¹² is H,

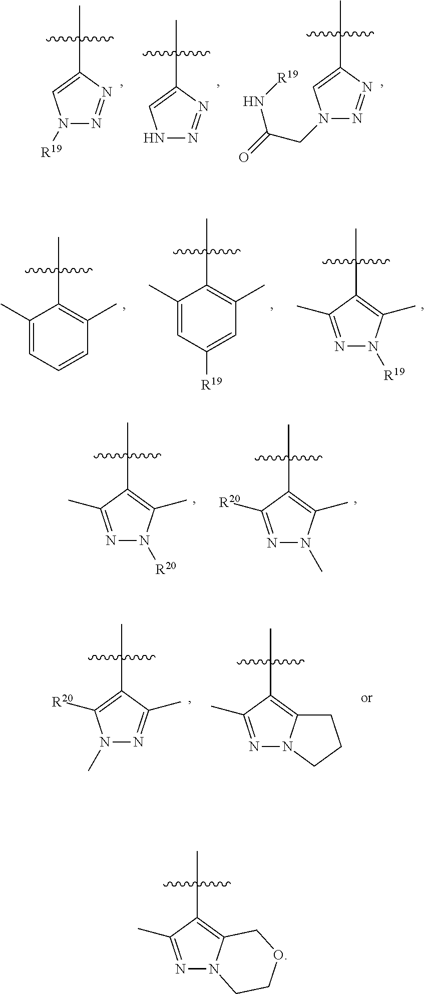

34. The compound of any preceding claim, wherein R²⁰ is Me, —CH₂—O-bromobenzaldehyde, or

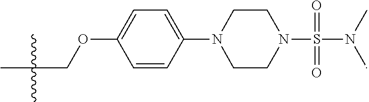

35. The compound of any preceding claim wherein when R⁸ is H, R¹³ is

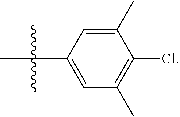 [SMILES: *c1cc(C)c(Cl)c(C)c1]

36. The compound of any preceding claim, wherein
R⁸ is H, R¹⁹, methyl, or —CH₂CH₂-morpholine;
R⁹ is —C(O)OH or —C(O)NHR¹⁹,
R¹⁰ is —C₃H₆O—R¹³,
wherein R¹³ is 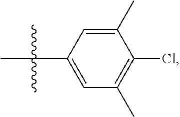 [SMILES: *c1cc(C)c(Cl)c(C)c1]
tetraline, or naphthyl optionally substituted with fluorine;
R¹¹ is H, Cl, F or methyl,
R¹² is 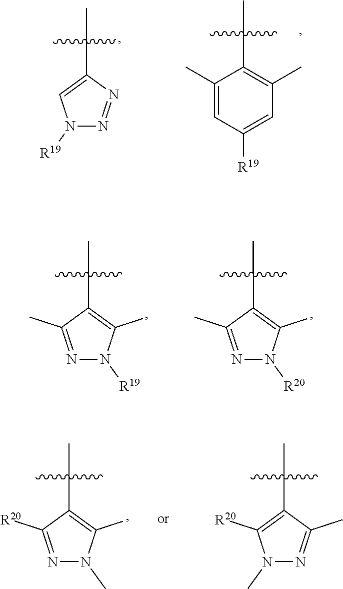
wherein R²⁰ is Me, —CH₂—O-bromobenzaldehyde, or

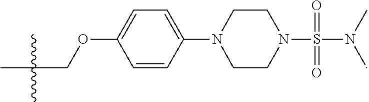

37. The compound of any preceding claim wherein Z₂ is N and [symbol] is a single bond.

38. The compound of any one of claims 1-36 wherein Z₂ is C and [symbol] is a double bond.

39. The compound of any preceding claim, wherein R¹¹ is hydrogen.

40. The compound of any one of claims 1-38, wherein R¹¹ is halogen or C₁-C₆ alkyl.

41. The compound of claim 40, wherein R¹¹ is halogen.

42. The compound of any preceding claim, wherein [MCL-1 ligand moiety] is selected from:

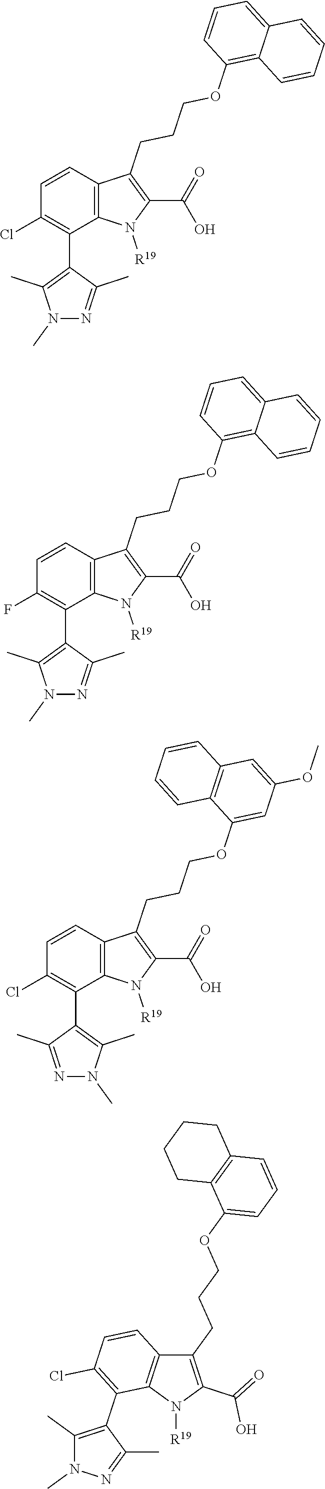

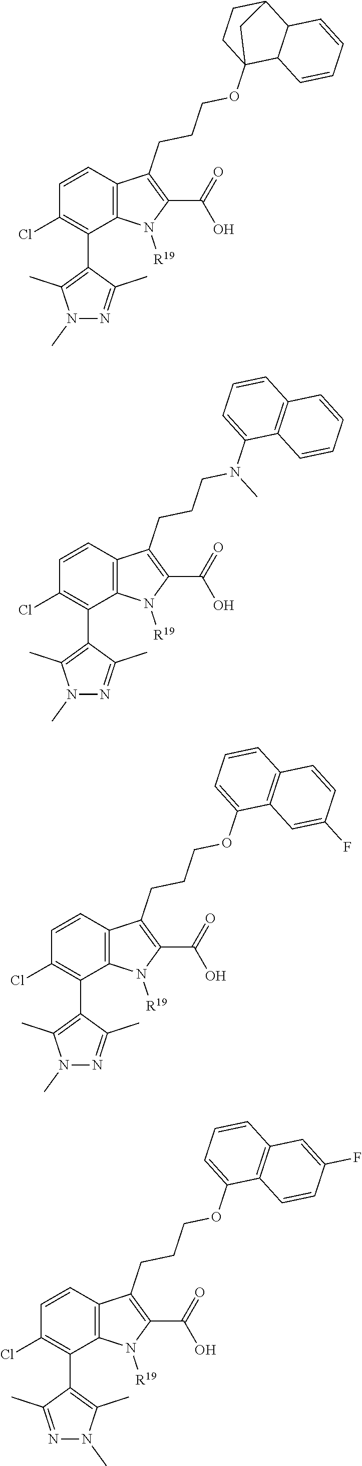

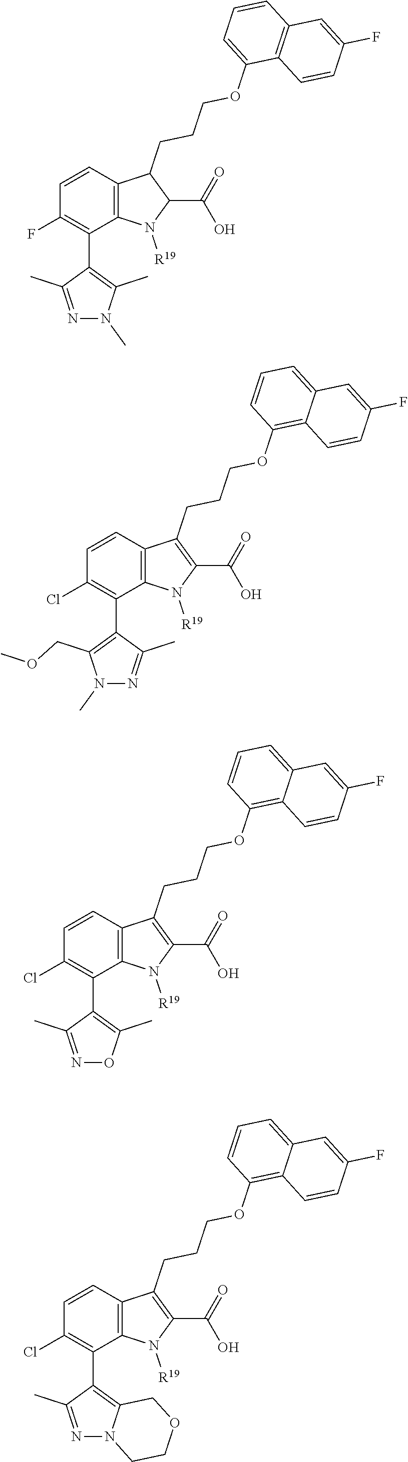

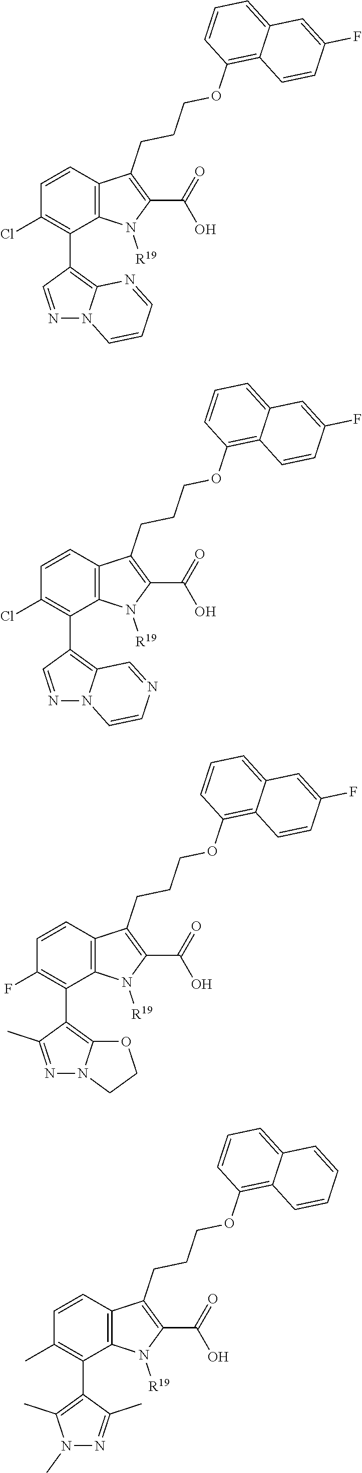

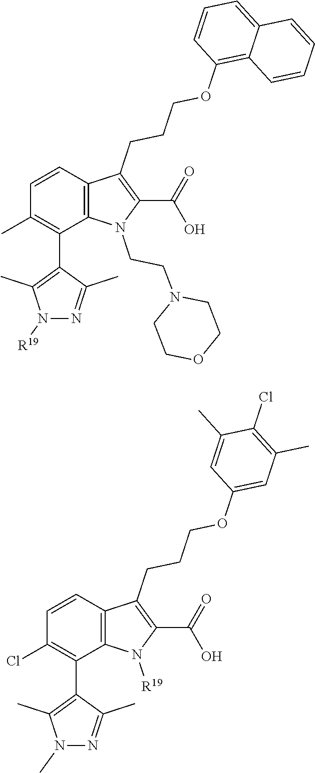

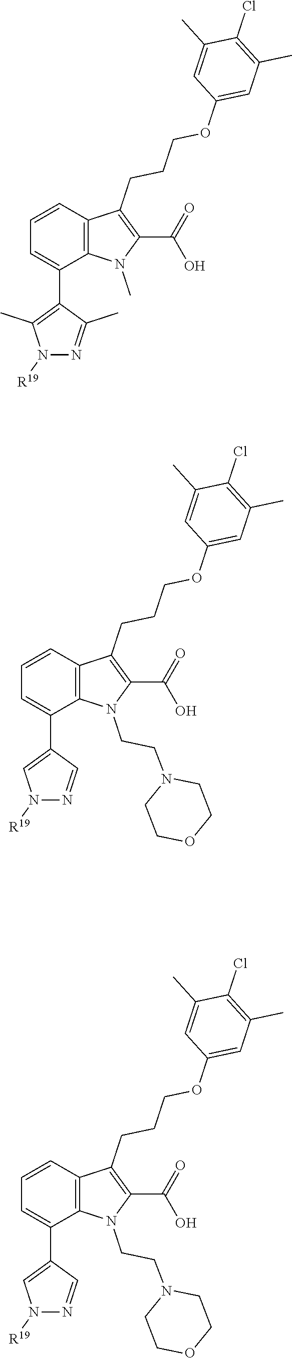

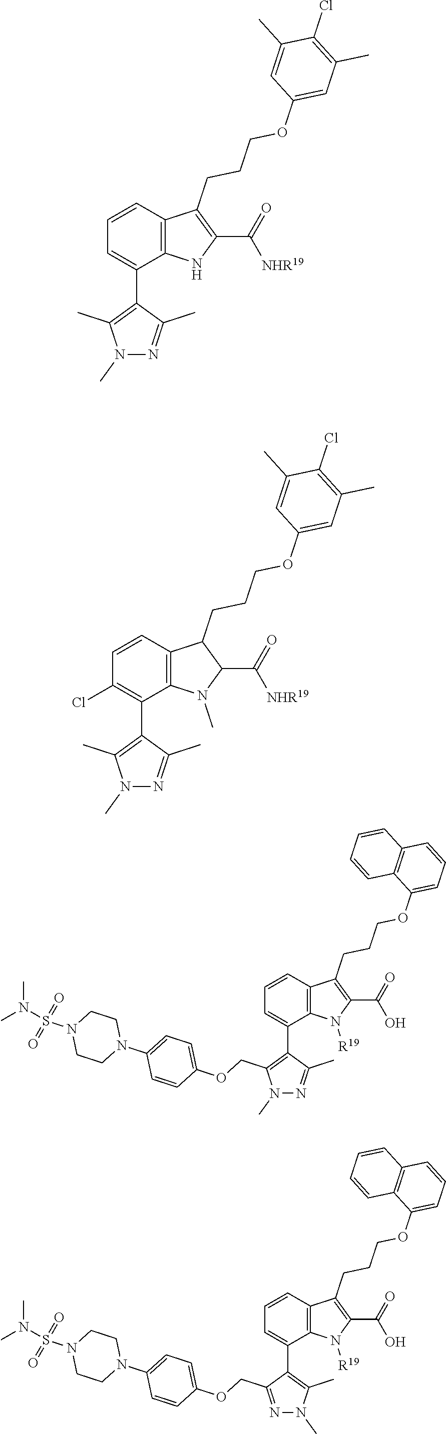

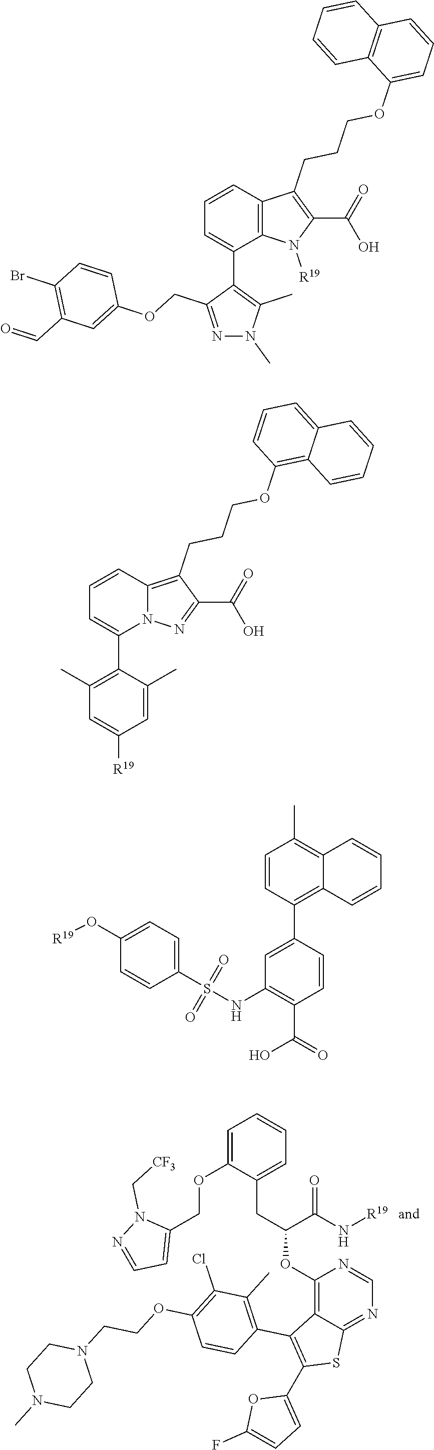

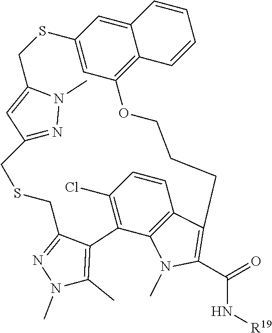

43. The compound of claim 1, which is selected from:

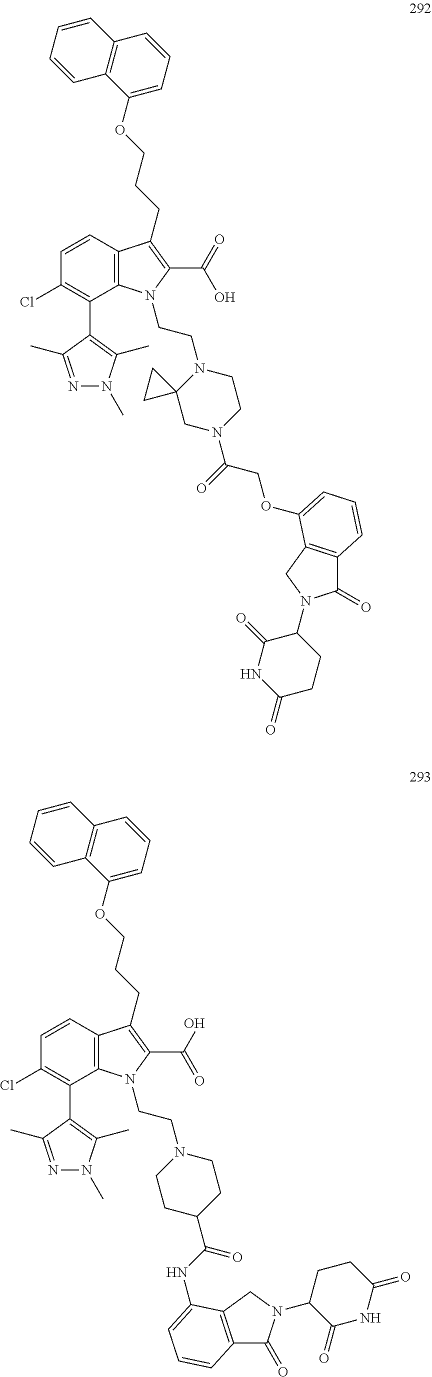 [SMILES: Cc1nn(C)c(C)c1-c1c(Cl)ccc2c(CCCOc3cccc4ccccc34)c(C(=O)O)n(CCN3CCN(C(=O)COc4cccc5c4CN(C4CCC(=O)NC4=O)C5=O)CC34CC4)c12 ; Cc1nn(C)c(C)c1-c1c(Cl)ccc2c(CCCOc3cccc4ccccc34)c(C(=O)O)n(CCN3CCC(C(=O)Nc4cccc5c4CN(C4CCC(=O)NC4=O)C5=O)CC3)c12]

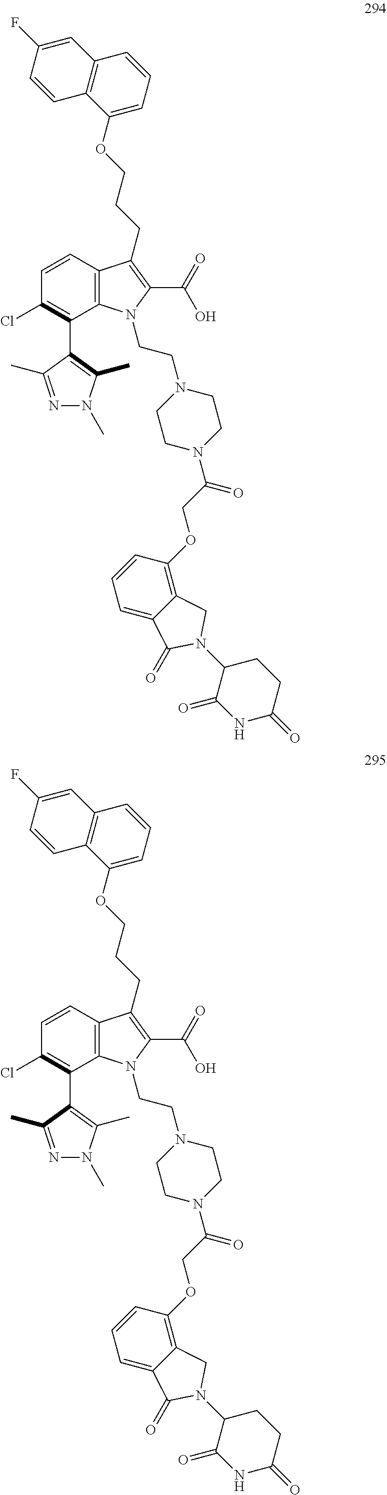

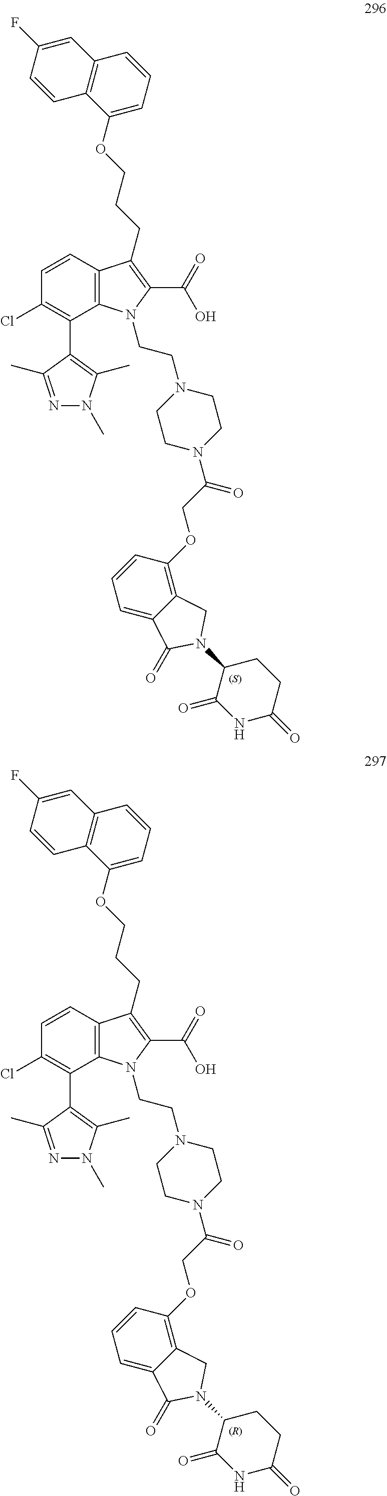

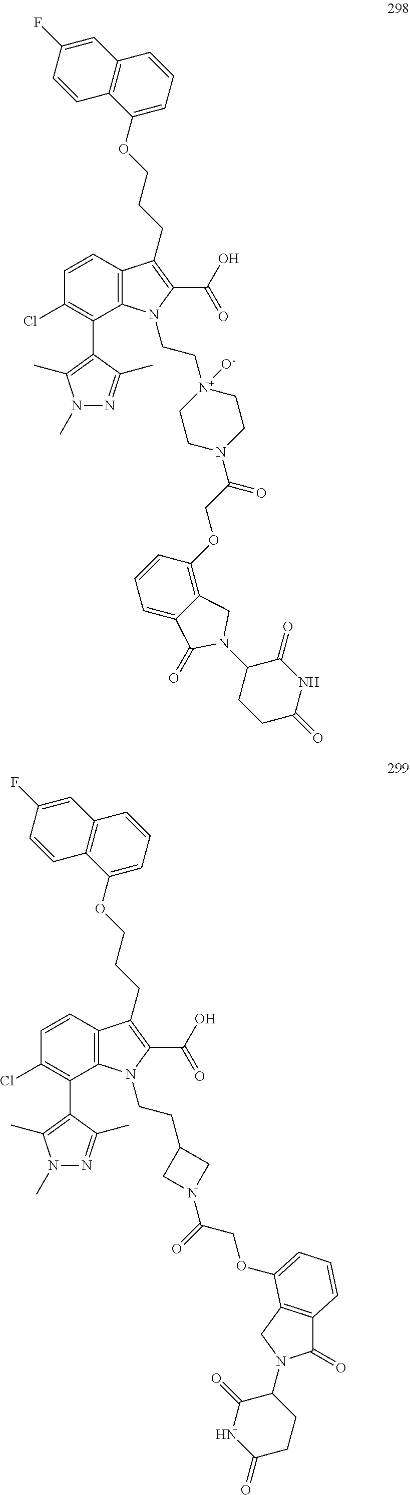 [SMILES: Cc1nn(C)c(C)c1-c1c(Cl)ccc2c(CCCOc3cccc4cc(F)ccc34)c(C(=O)O)n(CC[N+]3([O-])CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCC3CN(C(=O)COc4cccc5c4CN(C4CCC(=O)NC4=O)C5=O)C3)c12]

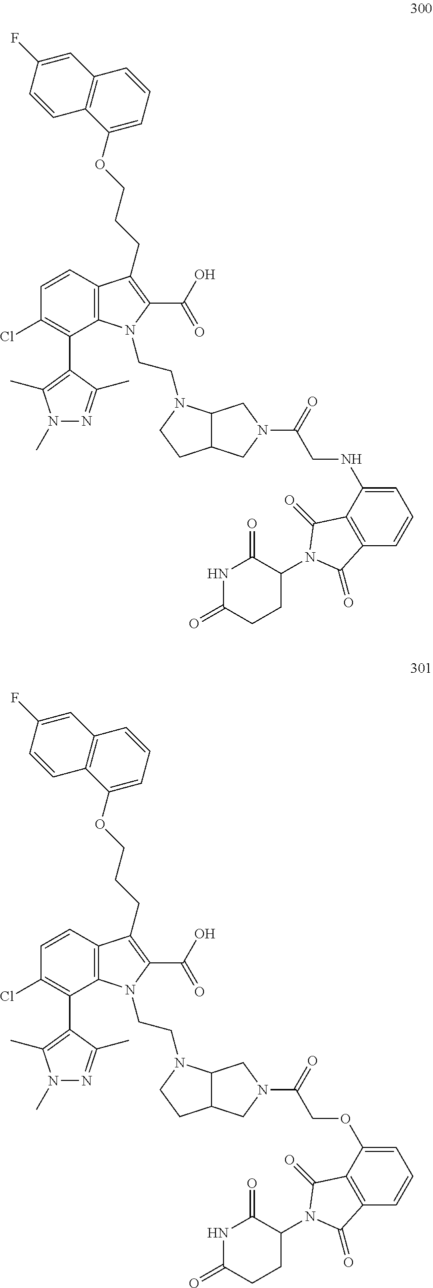 [SMILES: Cc1nn(C)c(C)c1-c1c(Cl)ccc2c(CCCOc3cccc4cc(F)ccc34)c(C(=O)O)n(CCN3CCC4CN(C(=O)CNc5cccc6c5C(=O)N(C5CCC(=O)NC5=O)C6=O)CC43)c12 ; Cc1nn(C)c(C)c1-c1c(Cl)ccc2c(CCCOc3cccc4cc(F)ccc34)c(C(=O)O)n(CCN3CCC4CN(C(=O)COc5cccc6c5C(=O)N(C5CCC(=O)NC5=O)C6=O)CC43)c12]

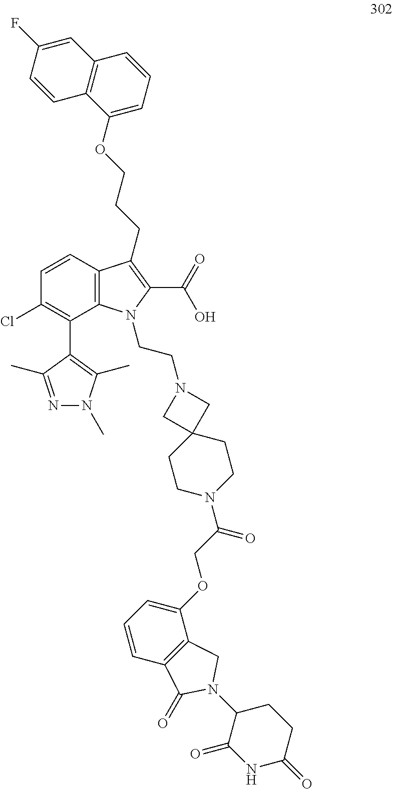 [SMILES: Cc1nn(C)c(C)c1-c1c(Cl)ccc2c(CCCOc3cccc4cc(F)ccc34)c(C(=O)O)n(CCN3CC4(CCN(C(=O)COc5cccc6c5CN(C5CCC(=O)NC5=O)C6=O)CC4)C3)c12]

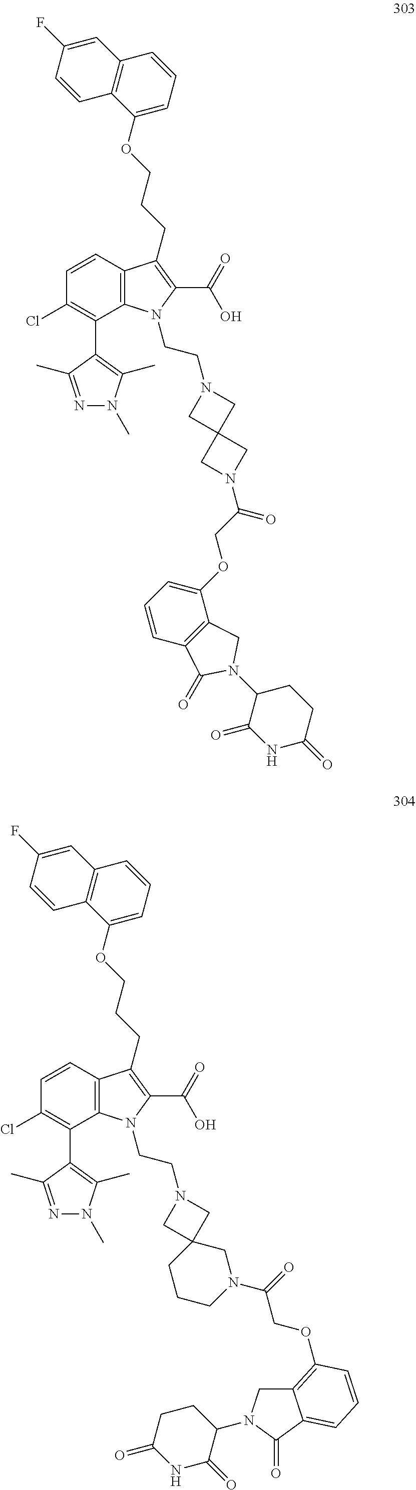 [SMILES: Cc1nn(C)c(C)c1-c1c(Cl)ccc2c(CCCOc3cccc4cc(F)ccc34)c(C(=O)O)n(CCN3CC4(C3)CN(C(=O)COc3cccc5c3CN(C3CCC(=O)NC3=O)C5=O)C4)c12 ; Cc1nn(C)c(C)c1-c1c(Cl)ccc2c(CCCOc3cccc4cc(F)ccc34)c(C(=O)O)n(CCN3CC4(CCCN(C(=O)COc5cccc6c5CN(C5CCC(=O)NC5=O)C6=O)C4)C3)c12]

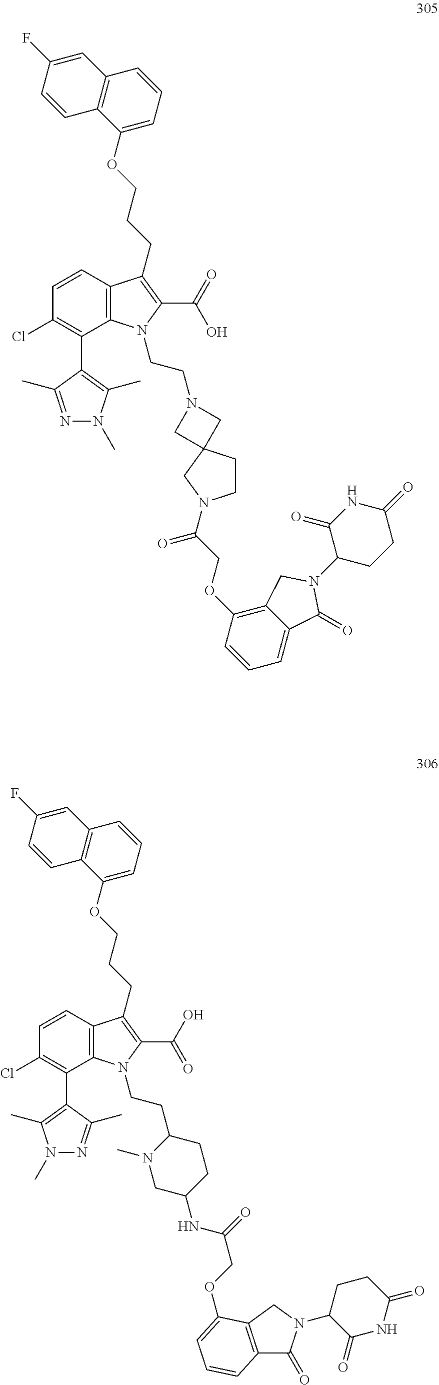 [SMILES: Cc1nn(C)c(C)c1-c1c(Cl)ccc2c(CCCOc3cccc4cc(F)ccc34)c(C(=O)O)n(CCN3CC4(CCN(C(=O)COc5cccc6c5CN(C5CCC(=O)NC5=O)C6=O)C4)C3)c12 ; Cc1nn(C)c(C)c1-c1c(Cl)ccc2c(CCCOc3cccc4cc(F)ccc34)c(C(=O)O)n(CCC3CCC(NC(=O)COc4cccc5c4CN(C4CCC(=O)NC4=O)C5=O)CN3C)c12]

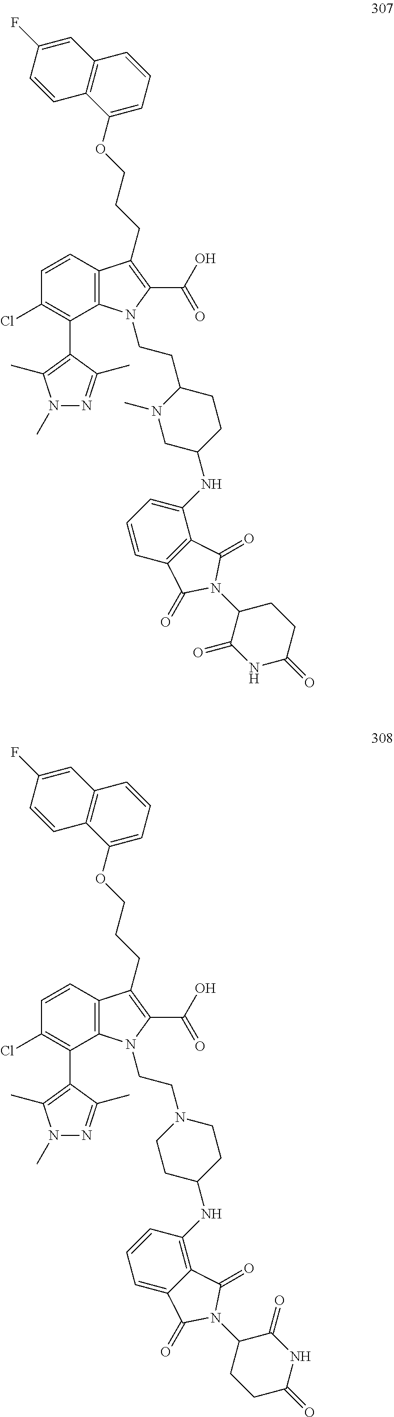 [SMILES: Cc1nn(C)c(C)c1-c1c(Cl)ccc2c(CCCOc3cccc4cc(F)ccc34)c(C(=O)O)n(CCC3CCC(Nc4cccc5c4C(=O)N(C4CCC(=O)NC4=O)C5=O)CN3C)c12 ; Cc1nn(C)c(C)c1-c1c(Cl)ccc2c(CCCOc3cccc4cc(F)ccc34)c(C(=O)O)n(CCN3CCC(Nc4cccc5c4C(=O)N(C4CCC(=O)NC4=O)C5=O)CC3)c12]

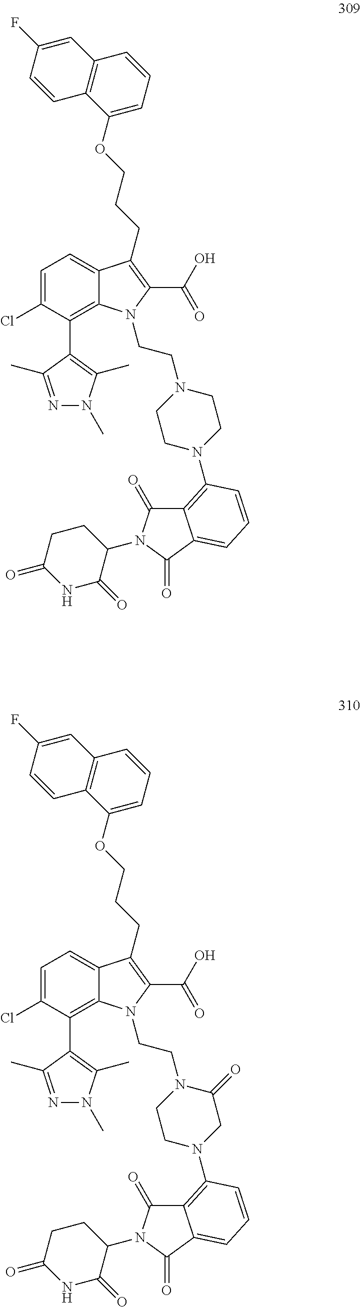 [SMILES: Cc1nn(C)c(C)c1-c1c(Cl)ccc2c(CCCOc3cccc4cc(F)ccc34)c(C(=O)O)n(CCN3CCN(c4cccc5c4C(=O)N(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(c4cccc5c4C(=O)N(C4CCC(=O)NC4=O)C5=O)CC3=O)c12]

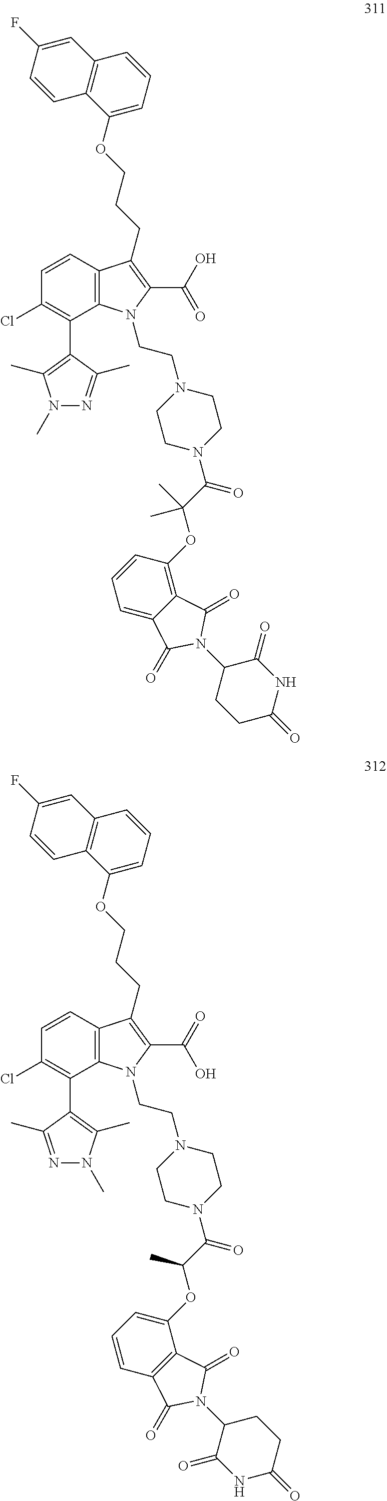 [SMILES: Cc1nn(C)c(C)c1-c1c(Cl)ccc2c(CCCOc3cccc4cc(F)ccc34)c(C(=O)O)n(CCN3CCN(C(=O)C(C)(C)Oc4cccc5c4C(=O)N(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)[C@H](C)Oc4cccc5c4C(=O)N(C4CCC(=O)NC4=O)C5=O)CC3)c12]

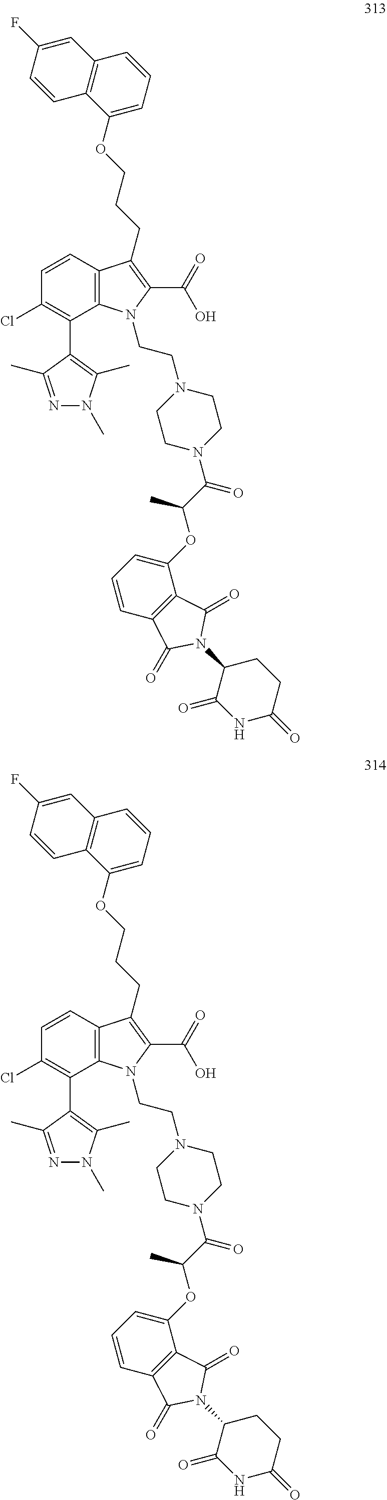 [SMILES: Cc1nn(C)c(C)c1-c1c(Cl)ccc2c(CCCOc3cccc4cc(F)ccc34)c(C(=O)O)n(CCN3CCN(C(=O)[C@H](C)Oc4cccc5c4C(=O)N([C@H]4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)[C@H](C)Oc4cccc5c4C(=O)N([C@@H]4CCC(=O)NC4=O)C5=O)CC3)c12]

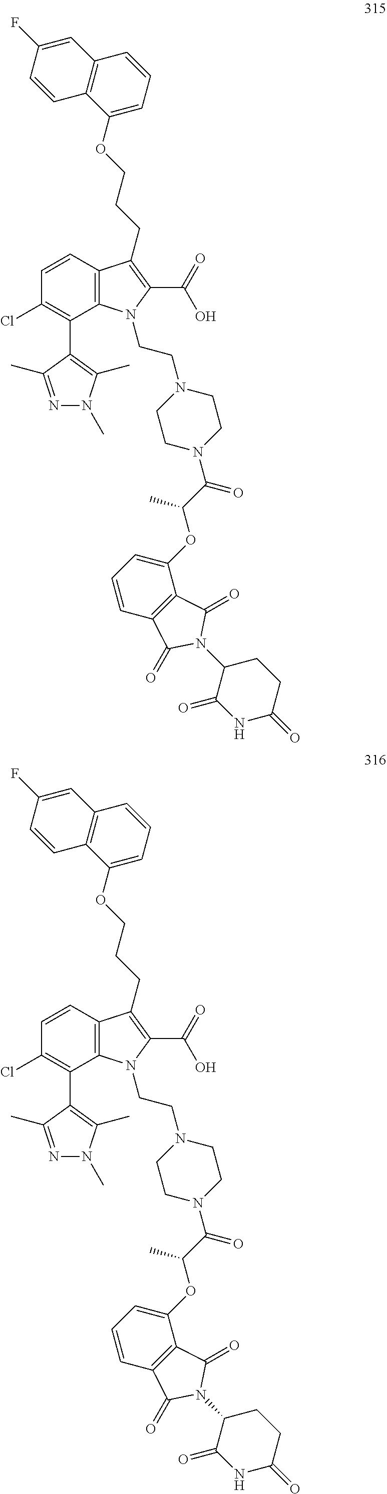 [SMILES: Cc1nn(C)c(C)c1-c1c(Cl)ccc2c(CCCOc3cccc4cc(F)ccc34)c(C(=O)O)n(CCN3CCN(C(=O)[C@@H](C)Oc4cccc5c4C(=O)N(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)[C@@H](C)Oc4cccc5c4C(=O)N([C@@H]4CCC(=O)NC4=O)C5=O)CC3)c12]

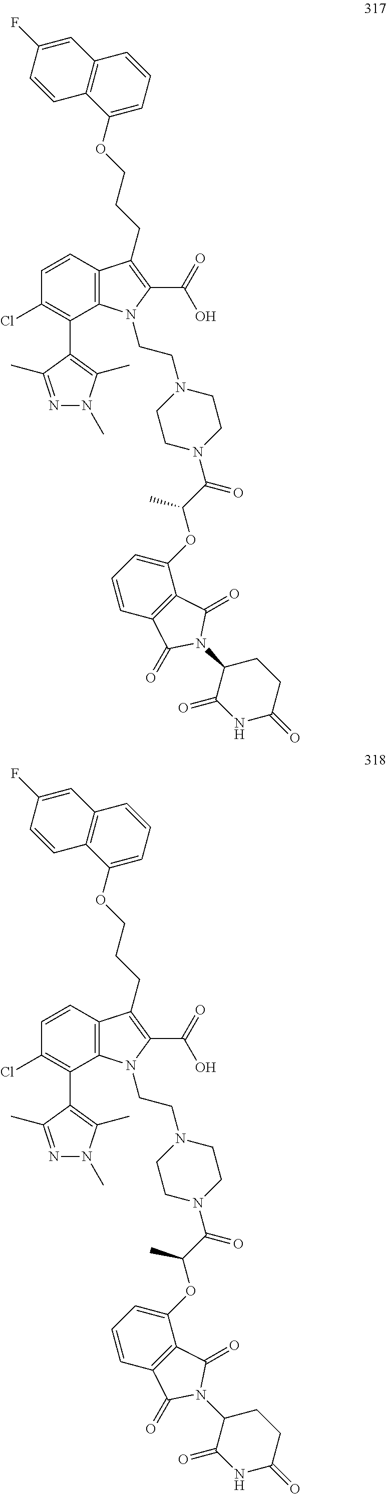 [SMILES: Cc1nn(C)c(C)c1-c1c(Cl)ccc2c(CCCOc3cccc4cc(F)ccc34)c(C(=O)O)n(CCN3CCN(C(=O)[C@@H](C)Oc4cccc5c4C(=O)N([C@H]4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)[C@H](C)Oc4cccc5c4C(=O)N(C4CCC(=O)NC4=O)C5=O)CC3)c12]

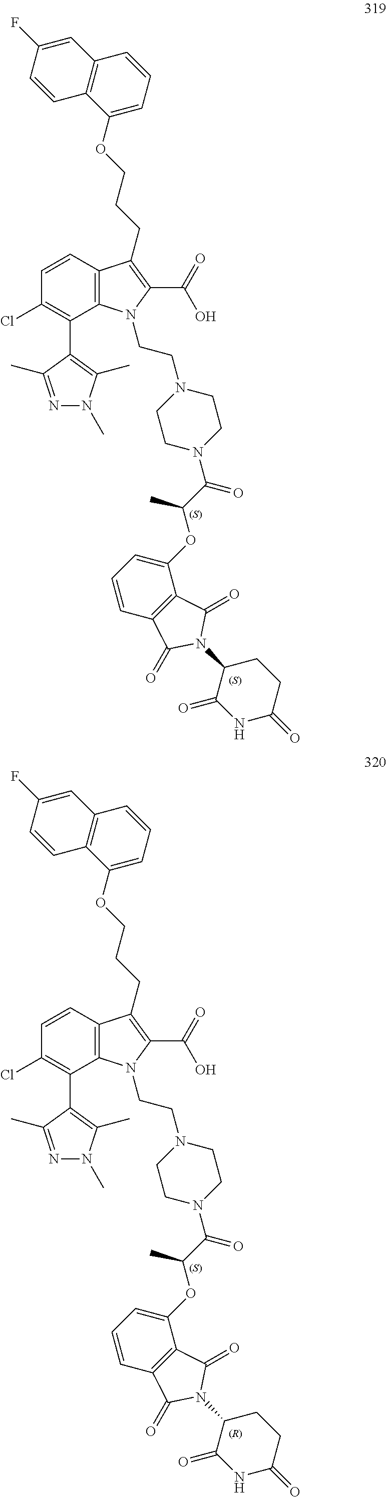

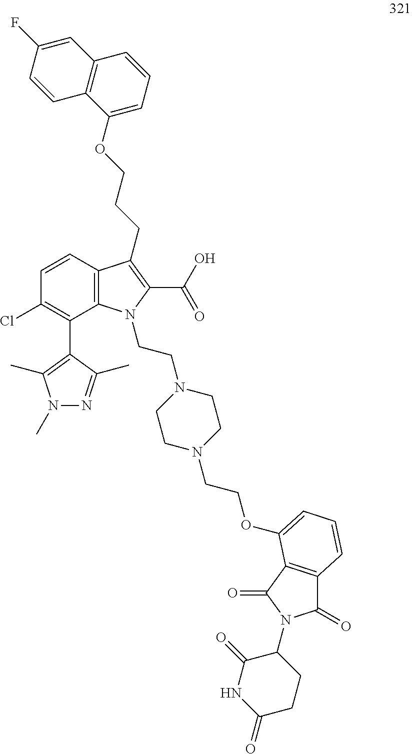 [SMILES: Cc1nn(C)c(C)c1-c1c(Cl)ccc2c(CCCOc3cccc4cc(F)ccc34)c(C(=O)O)n(CCN3CCN(CCOc4cccc5c4C(=O)N(C4CCC(=O)NC4=O)C5=O)CC3)c12]

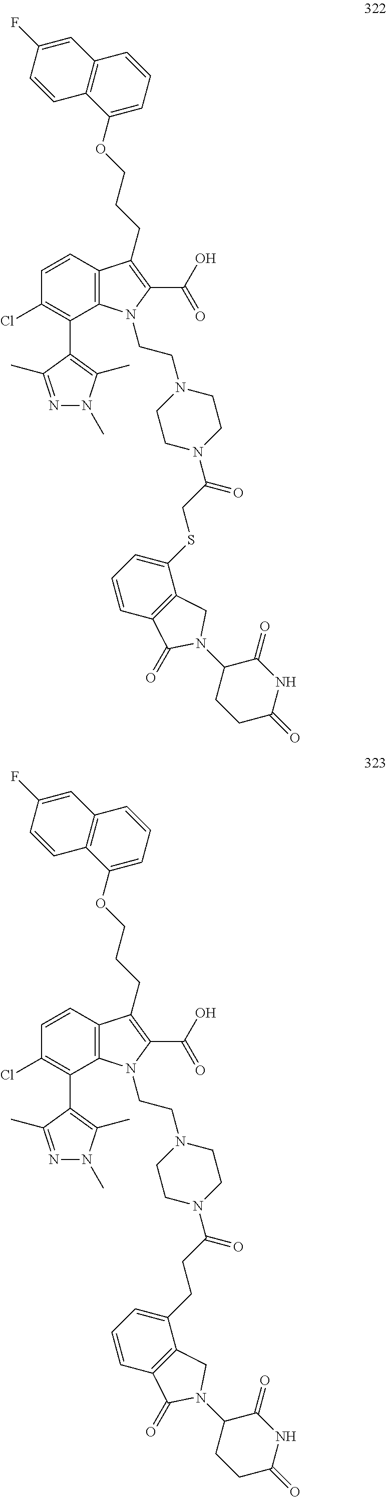 [SMILES: Cc1nn(C)c(C)c1-c1c(Cl)ccc2c(CCCOc3cccc4cc(F)ccc34)c(C(=O)O)n(CCN3CCN(C(=O)CSc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)CCc4cccc5c4CN(C4CCC(=O)NC4=O)C5=O)CC3)c12]

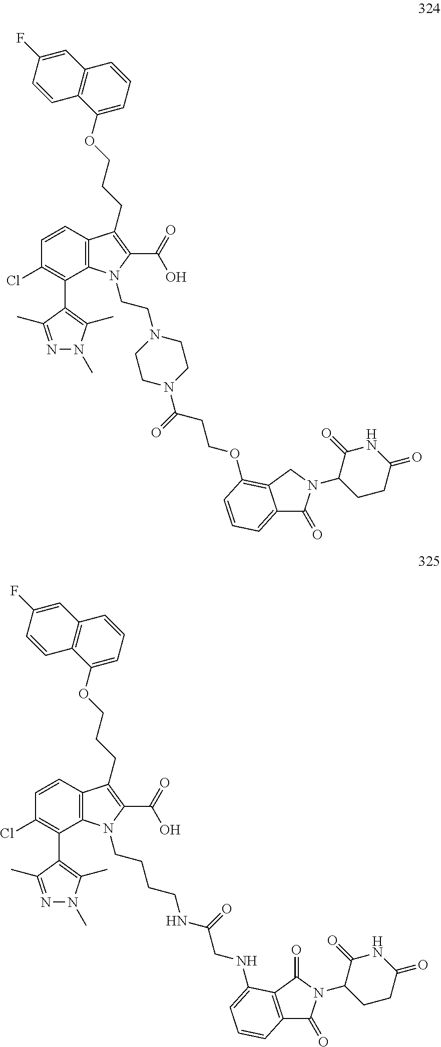 [SMILES: Cc1nn(C)c(C)c1-c1c(Cl)ccc2c(CCCOc3cccc4cc(F)ccc34)c(C(=O)O)n(CCN3CCN(C(=O)CCOc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCCCNC(=O)CNc3cccc4c3C(=O)N(C3CCC(=O)NC3=O)C4=O)c12]

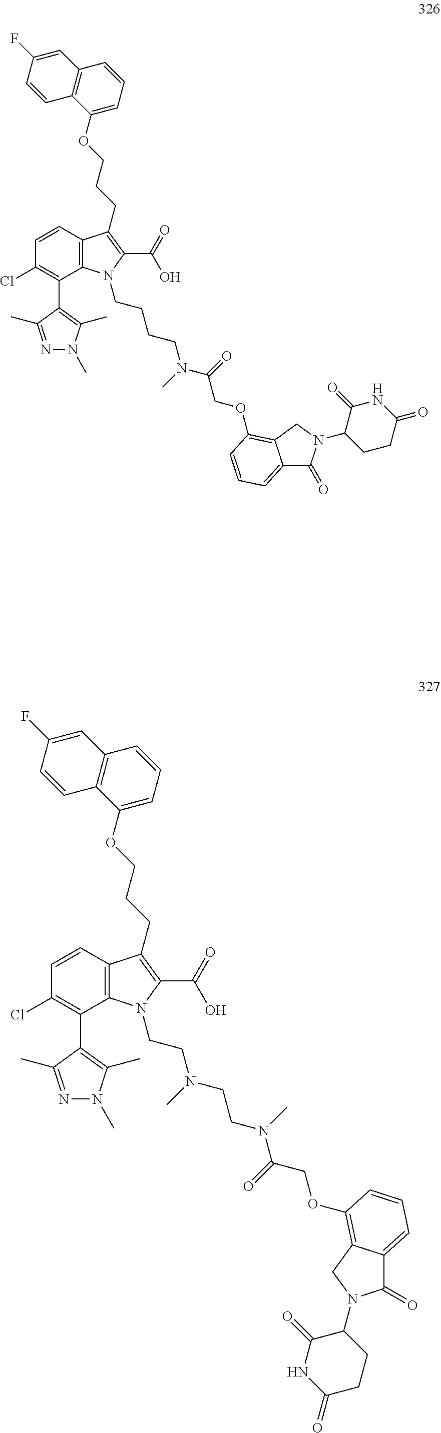 [SMILES: Cc1nn(C)c(C)c1-c1c(Cl)ccc2c(CCCOc3cccc4cc(F)ccc34)c(C(=O)O)n(CCCCN(C)C(=O)COc3cccc4c3CN(C3CCC(=O)NC3=O)C4=O)c12 ; Cc1nn(C)c(C)c1-c1c(Cl)ccc2c(CCCOc3cccc4cc(F)ccc34)c(C(=O)O)n(CCN(C)CCN(C)C(=O)COc3cccc4c3CN(C3CCC(=O)NC3=O)C4=O)c12]

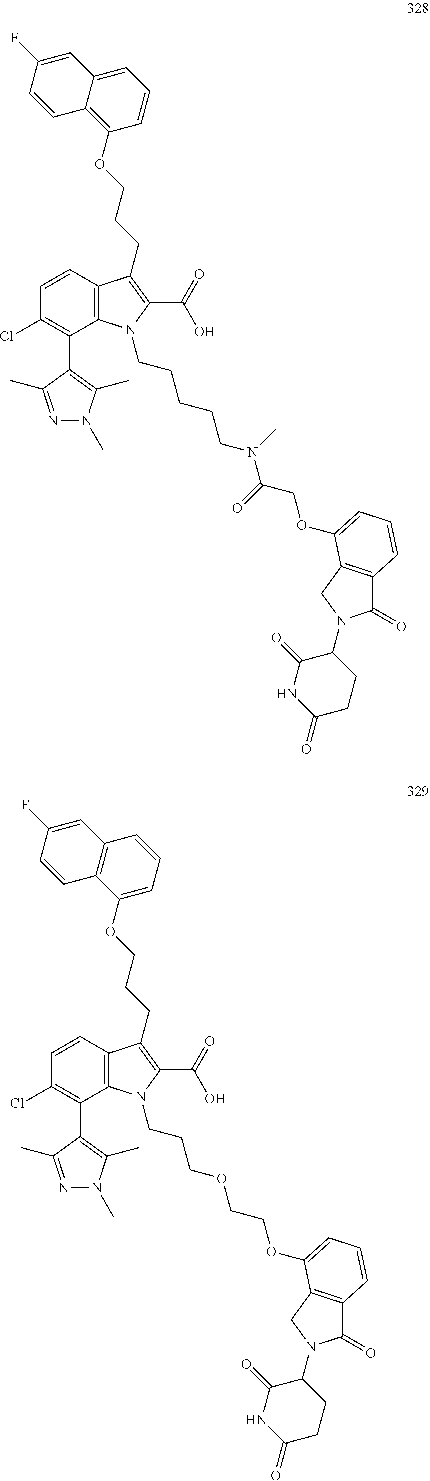 [SMILES: Cc1nn(C)c(C)c1-c1c(Cl)ccc2c(CCCOc3cccc4cc(F)ccc34)c(C(=O)O)n(CCCCCN(C)C(=O)COc3cccc4c3CN(C3CCC(=O)NC3=O)C4=O)c12 ; Cc1nn(C)c(C)c1-c1c(Cl)ccc2c(CCCOc3cccc4cc(F)ccc34)c(C(=O)O)n(CCCOCCOc3cccc4c3CN(C3CCC(=O)NC3=O)C4=O)c12]

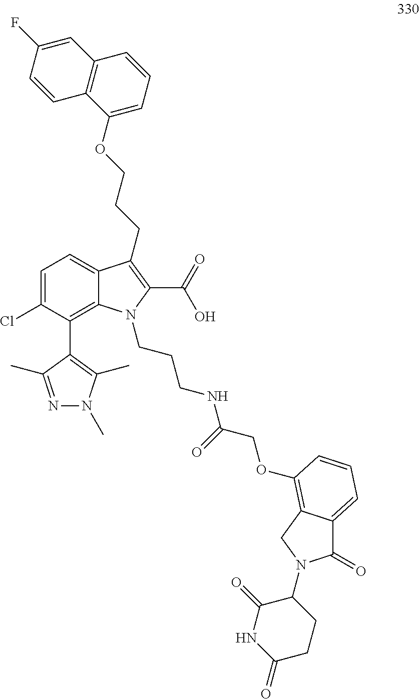 [SMILES: Cc1nn(C)c(C)c1-c1c(Cl)ccc2c(CCCOc3cccc4cc(F)ccc34)c(C(=O)O)n(CCCNC(=O)COc3cccc4c3CN(C3CCC(=O)NC3=O)C4=O)c12]

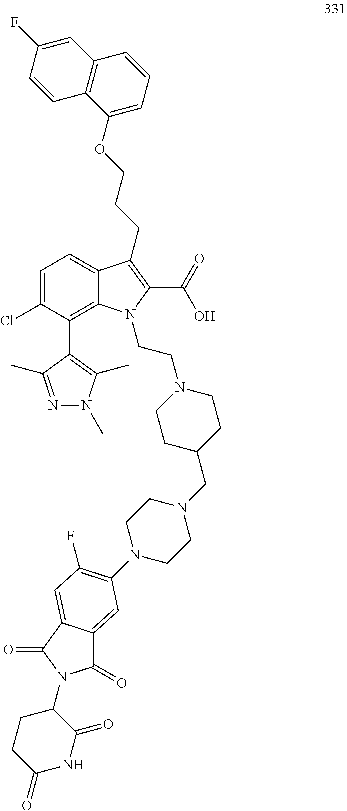 [SMILES: Cc1nn(C)c(C)c1-c1c(Cl)ccc2c(CCCOc3cccc4cc(F)ccc34)c(C(=O)O)n(CCN3CCC(CN4CCN(c5cc6c(cc5F)C(=O)N(C5CCC(=O)NC5=O)C6=O)CC4)CC3)c12]

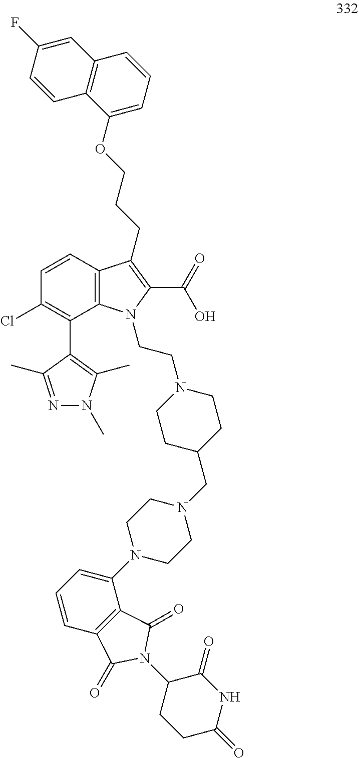 [SMILES: Cc1nn(C)c(C)c1-c1c(Cl)ccc2c(CCCOc3cccc4cc(F)ccc34)c(C(=O)O)n(CCN3CCC(CN4CCN(c5cccc6c5C(=O)N(C5CCC(=O)NC5=O)C6=O)CC4)CC3)c12]

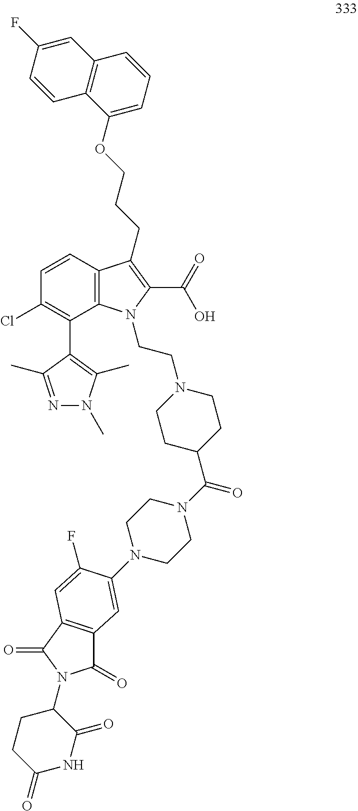 [SMILES: Cc1nn(C)c(C)c1-c1c(Cl)ccc2c(CCCOc3cccc4cc(F)ccc34)c(C(=O)O)n(CCN3CCC(C(=O)N4CCN(c5cc6c(cc5F)C(=O)N(C5CCC(=O)NC5=O)C6=O)CC4)CC3)c12]

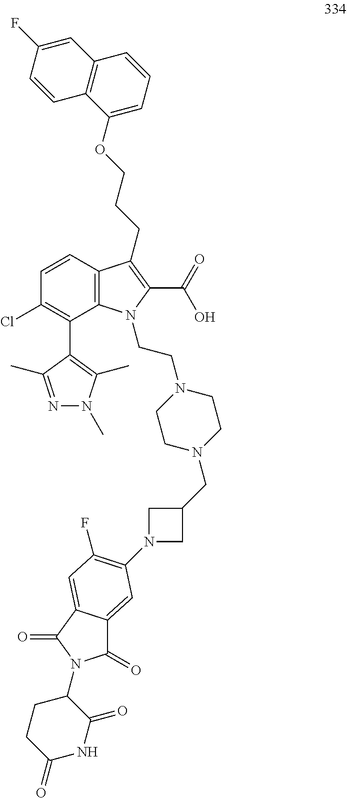 [SMILES: Cc1nn(C)c(C)c1-c1c(Cl)ccc2c(CCCOc3cccc4cc(F)ccc34)c(C(=O)O)n(CCN3CCN(CC4CN(c5cc6c(cc5F)C(=O)N(C5CCC(=O)NC5=O)C6=O)C4)CC3)c12]

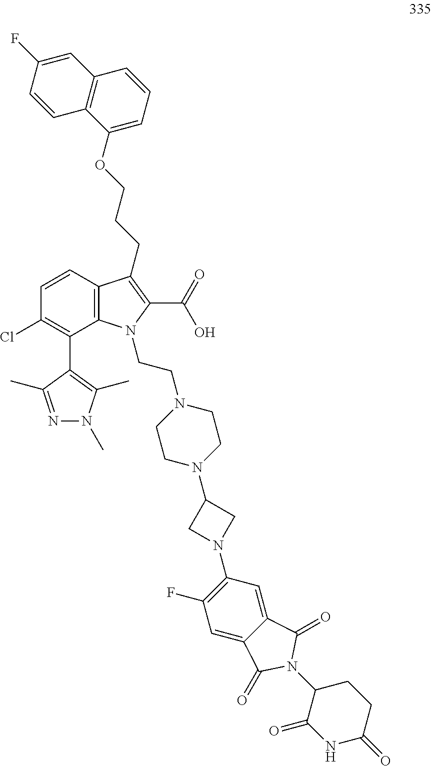 [SMILES: Cc1nn(C)c(C)c1-c1c(Cl)ccc2c(CCCOc3cccc4cc(F)ccc34)c(C(=O)O)n(CCN3CCN(C4CN(c5cc6c(cc5F)C(=O)N(C5CCC(=O)NC5=O)C6=O)C4)CC3)c12]

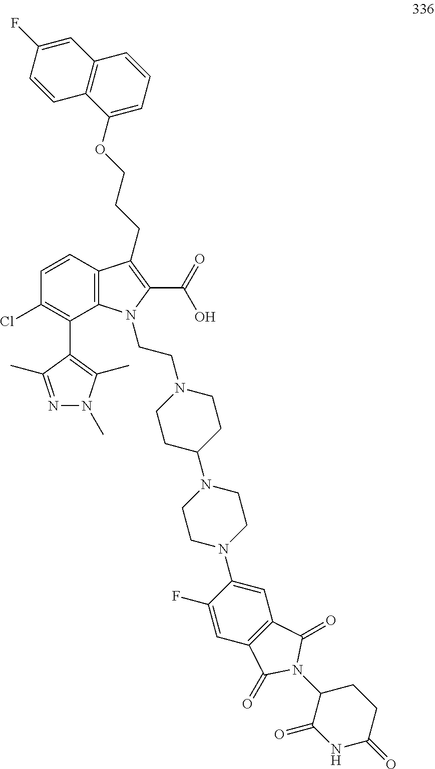 [SMILES: Cc1nn(C)c(C)c1-c1c(Cl)ccc2c(CCCOc3cccc4cc(F)ccc34)c(C(=O)O)n(CCN3CCC(N4CCN(c5cc6c(cc5F)C(=O)N(C5CCC(=O)NC5=O)C6=O)CC4)CC3)c12]

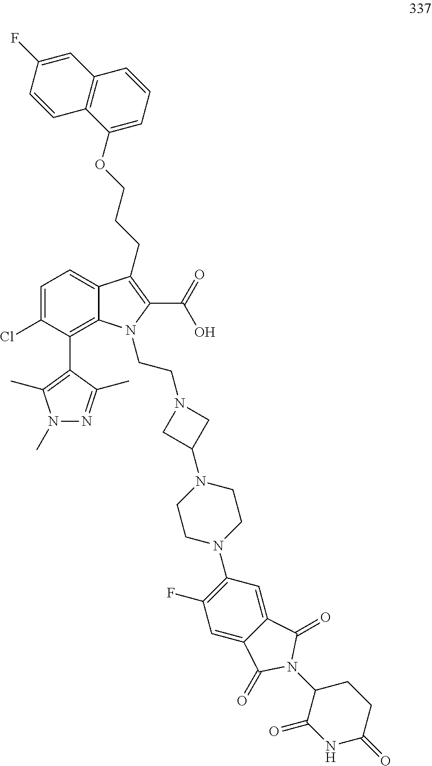 [SMILES: Cc1nn(C)c(C)c1-c1c(Cl)ccc2c(CCCOc3cccc4cc(F)ccc34)c(C(=O)O)n(CCN3CC(N4CCN(c5cc6c(cc5F)C(=O)N(C5CCC(=O)NC5=O)C6=O)CC4)C3)c12]

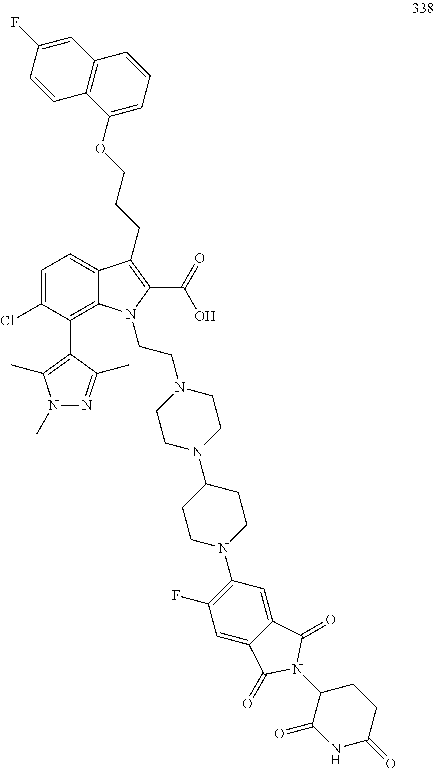 [SMILES: Cc1nn(C)c(C)c1-c1c(Cl)ccc2c(CCCOc3cccc4cc(F)ccc34)c(C(=O)O)n(CCN3CCN(C4CCN(c5cc6c(cc5F)C(=O)N(C5CCC(=O)NC5=O)C6=O)CC4)CC3)c12]

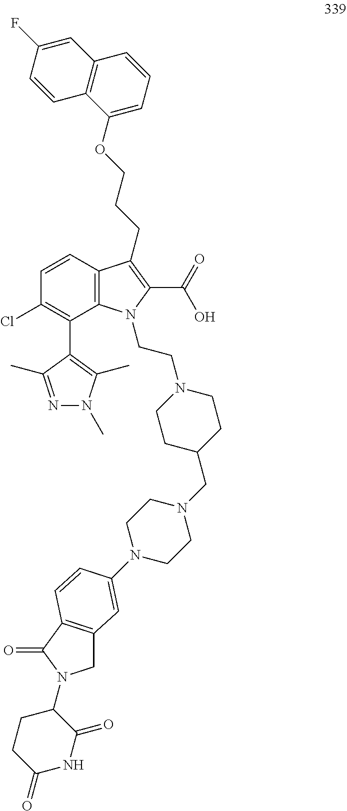 [SMILES: Cc1nn(C)c(C)c1-c1c(Cl)ccc2c(CCCOc3cccc4cc(F)ccc34)c(C(=O)O)n(CCN3CCC(CN4CCN(c5ccc6c(c5)CN(C5CCC(=O)NC5=O)C6=O)CC4)CC3)c12]

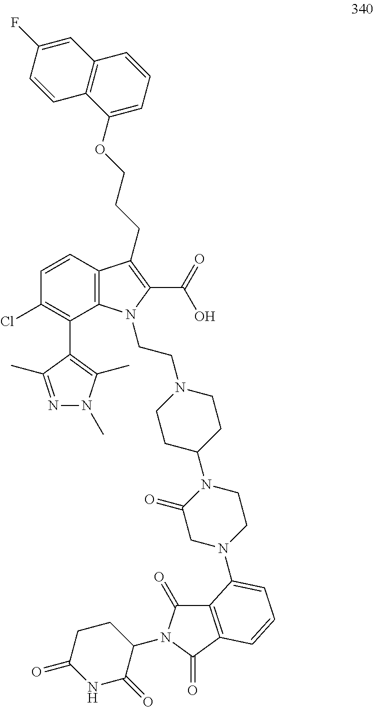 [SMILES: Cc1nn(C)c(C)c1-c1c(Cl)ccc2c(CCCOc3cccc4cc(F)ccc34)c(C(=O)O)n(CCN3CCC(N4CCN(c5cccc6c5C(=O)N(C5CCC(=O)NC5=O)C6=O)CC4=O)CC3)c12]

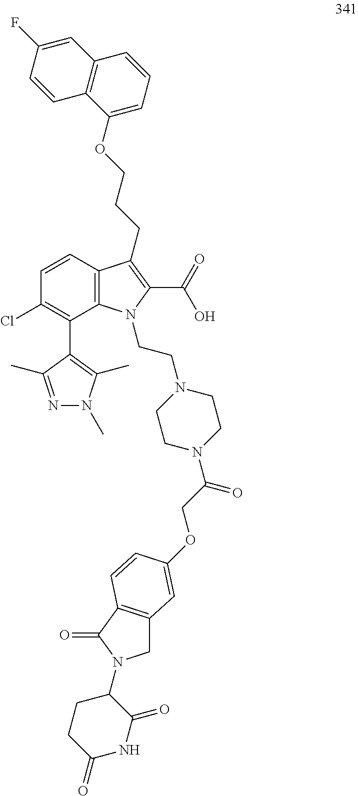 [SMILES: Cc1nn(C)c(C)c1-c1c(Cl)ccc2c(CCCOc3cccc4cc(F)ccc34)c(C(=O)O)n(CCN3CCN(C(=O)COc4ccc5c(c4)CN(C4CCC(=O)NC4=O)C5=O)CC3)c12]

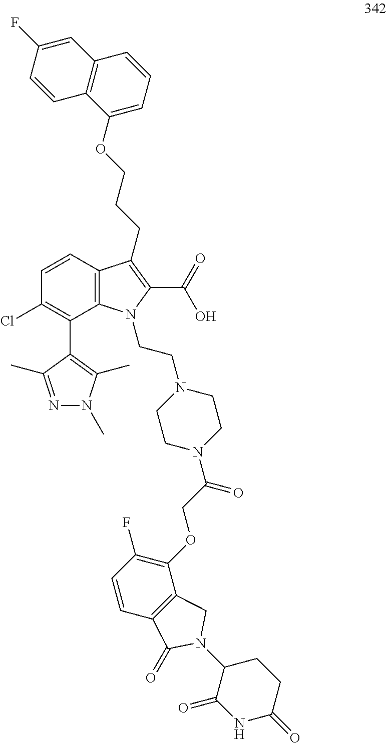 [SMILES: Cc1nn(C)c(C)c1-c1c(Cl)ccc2c(CCCOc3cccc4cc(F)ccc34)c(C(=O)O)n(CCN3CCN(C(=O)COc4c(F)ccc5c4CN(C4CCC(=O)NC4=O)C5=O)CC3)c12]

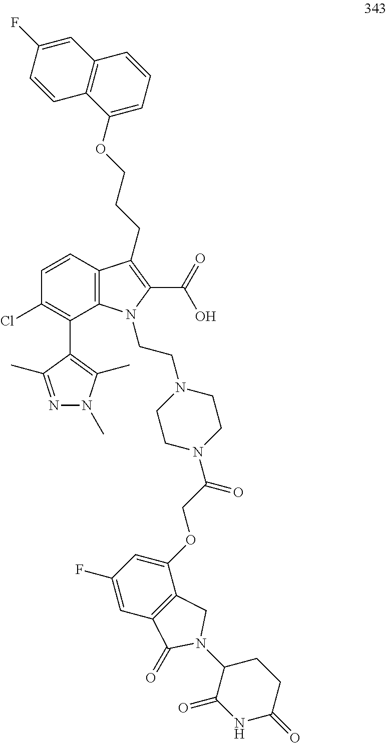 [SMILES: Cc1nn(C)c(C)c1-c1c(Cl)ccc2c(CCCOc3cccc4cc(F)ccc34)c(C(=O)O)n(CCN3CCN(C(=O)COc4cc(F)cc5c4CN(C4CCC(=O)NC4=O)C5=O)CC3)c12]

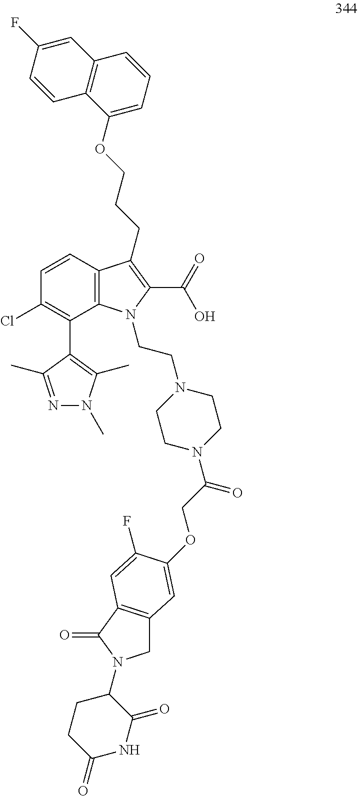 [SMILES: Cc1nn(C)c(C)c1-c1c(Cl)ccc2c(CCCOc3cccc4cc(F)ccc34)c(C(=O)O)n(CCN3CCN(C(=O)COc4cc5c(cc4F)C(=O)N(C4CCC(=O)NC4=O)C5)CC3)c12]

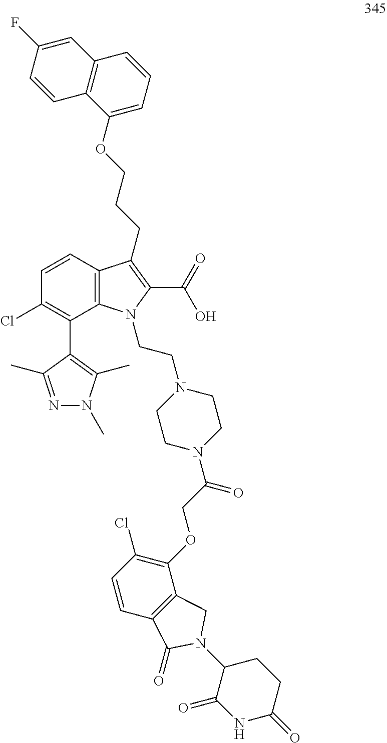 [SMILES: Cc1nn(C)c(C)c1-c1c(Cl)ccc2c(CCCOc3cccc4cc(F)ccc34)c(C(=O)O)n(CCN3CCN(C(=O)COc4c(Cl)ccc5c4CN(C4CCC(=O)NC4=O)C5=O)CC3)c12]

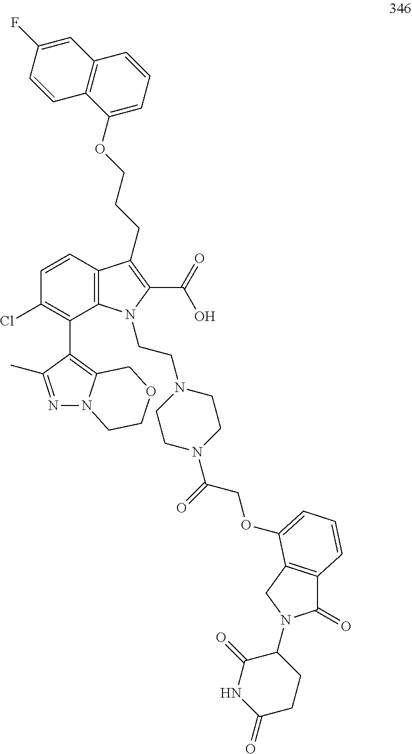 [SMILES: Cc1nn2c(c1-c1c(Cl)ccc3c(CCCOc4cccc5cc(F)ccc45)c(C(=O)O)n(CCN4CCN(C(=O)COc5cccc6c5CN(C5CCC(=O)NC5=O)C6=O)CC4)c13)COCC2]

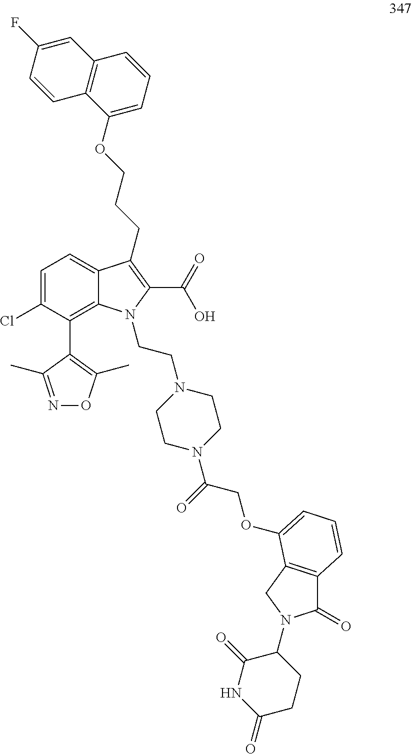 [SMILES: Cc1noc(C)c1-c1c(Cl)ccc2c(CCCOc3cccc4cc(F)ccc34)c(C(=O)O)n(CCN3CCN(C(=O)COc4cccc5c4CN(C4CCC(=O)NC4=O)C5=O)CC3)c12]

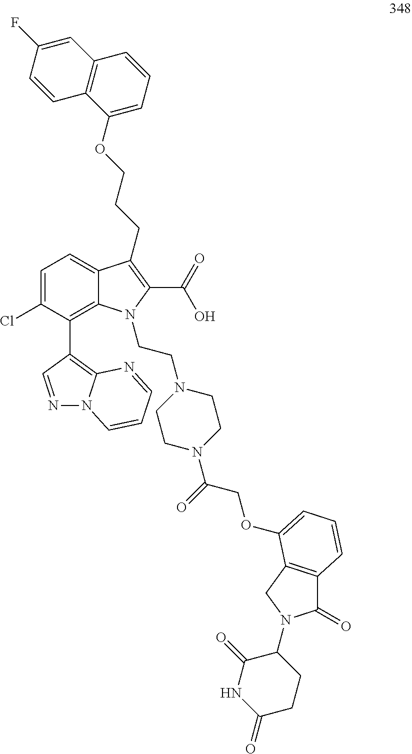 [SMILES: O=C1CCC(N2Cc3c(OCC(=O)N4CCN(CCn5c(C(=O)O)c(CCCOc6cccc7cc(F)ccc67)c6ccc(Cl)c(-c7cnn8cccnc78)c65)CC4)cccc3C2=O)C(=O)N1]

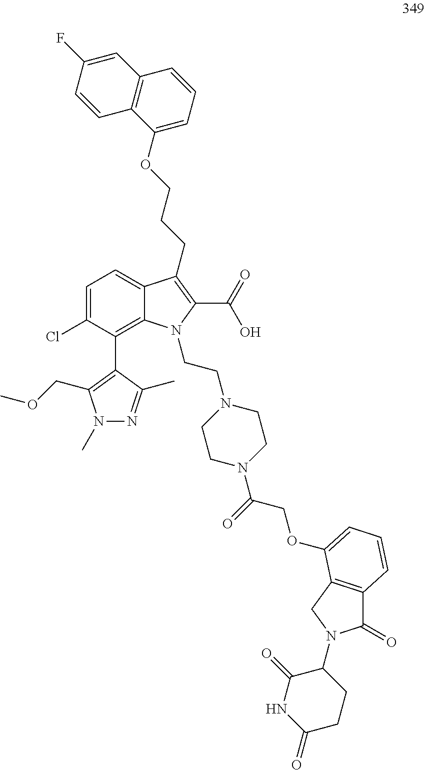 [SMILES: COCc1c(-c2c(Cl)ccc3c(CCCOc4cccc5cc(F)ccc45)c(C(=O)O)n(CCN4CCN(C(=O)COc5cccc6c5CN(C5CCC(=O)NC5=O)C6=O)CC4)c23)c(C)nn1C]

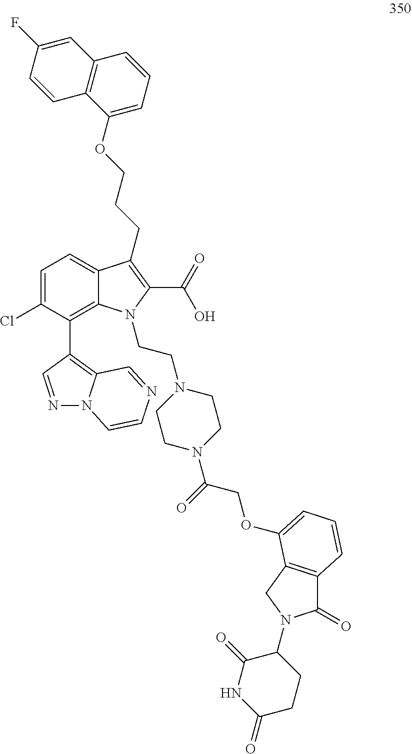 [SMILES: O=C1CCC(N2Cc3c(OCC(=O)N4CCN(CCn5c(C(=O)O)c(CCCOc6cccc7cc(F)ccc67)c6ccc(Cl)c(-c7cnn8ccncc78)c65)CC4)cccc3C2=O)C(=O)N1]

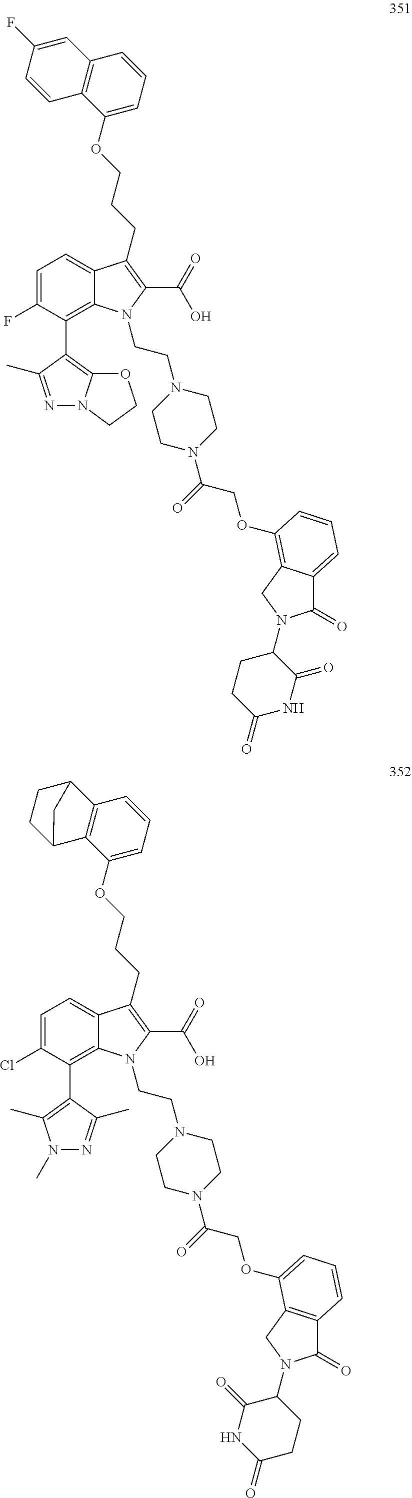 [SMILES: Cc1nn2c(c1-c1c(F)ccc3c(CCCOc4cccc5cc(F)ccc45)c(C(=O)O)n(CCN4CCN(C(=O)COc5cccc6c5CN(C5CCC(=O)NC5=O)C6=O)CC4)c13)OCC2 ; Cc1nn(C)c(C)c1-c1c(Cl)ccc2c(CCCOc3cccc4c3C3CCC4C3)c(C(=O)O)n(CCN3CCN(C(=O)COc4cccc5c4CN(C4CCC(=O)NC4=O)C5=O)CC3)c12]

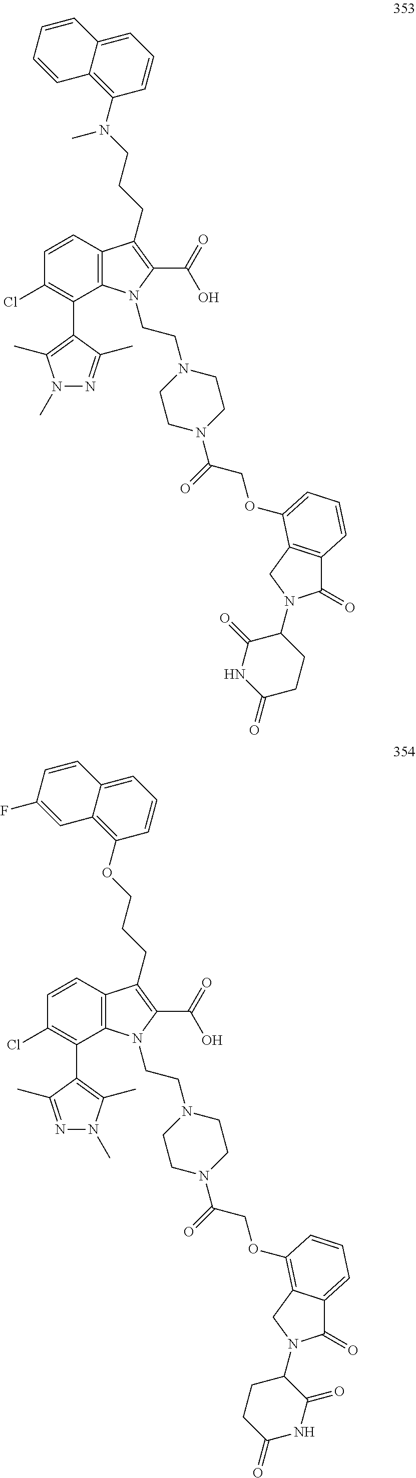 [SMILES: Cc1nn(C)c(C)c1-c1c(Cl)ccc2c(CCCN(C)c3cccc4ccccc34)c(C(=O)O)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4ccc(F)cc34)c(C(=O)O)n(CCN3CCN(C(=O)COc4cccc5c4CN(C4CCC(=O)NC4=O)C5=O)CC3)c12]

44. The compound of claim 1, which is selected from:

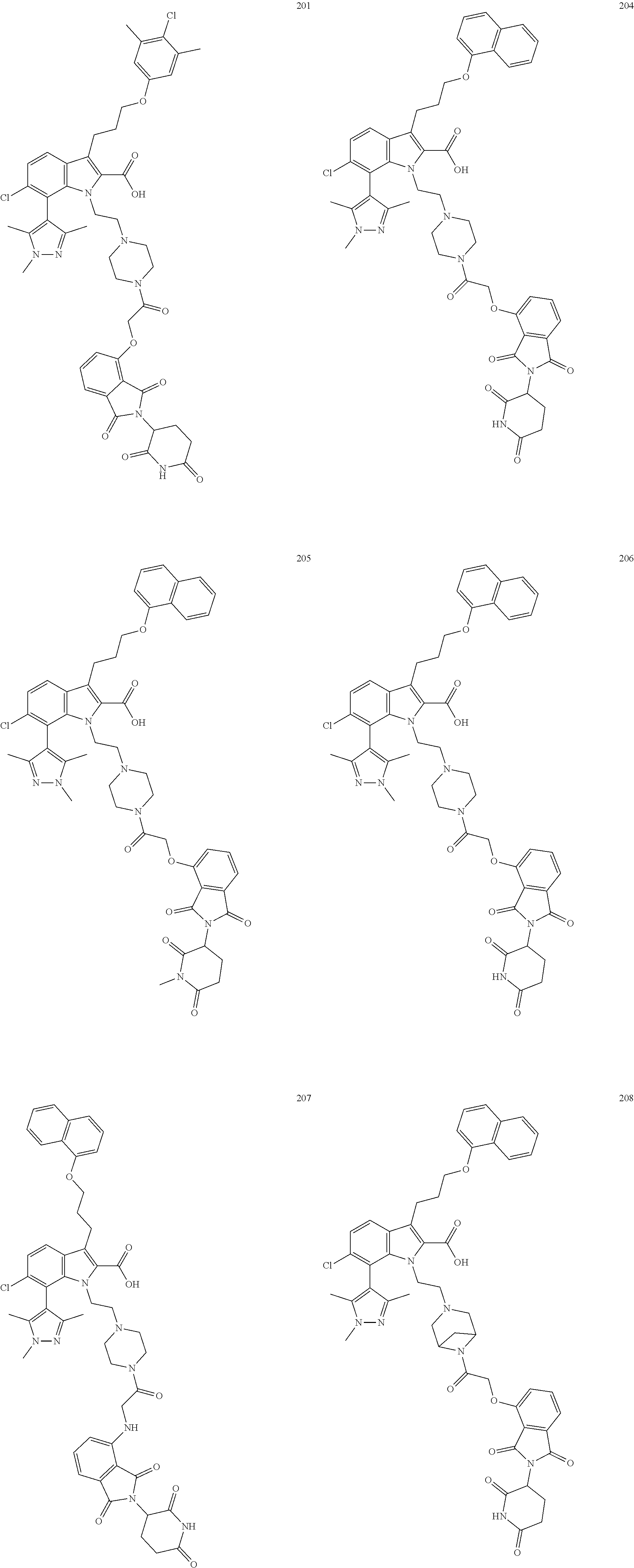

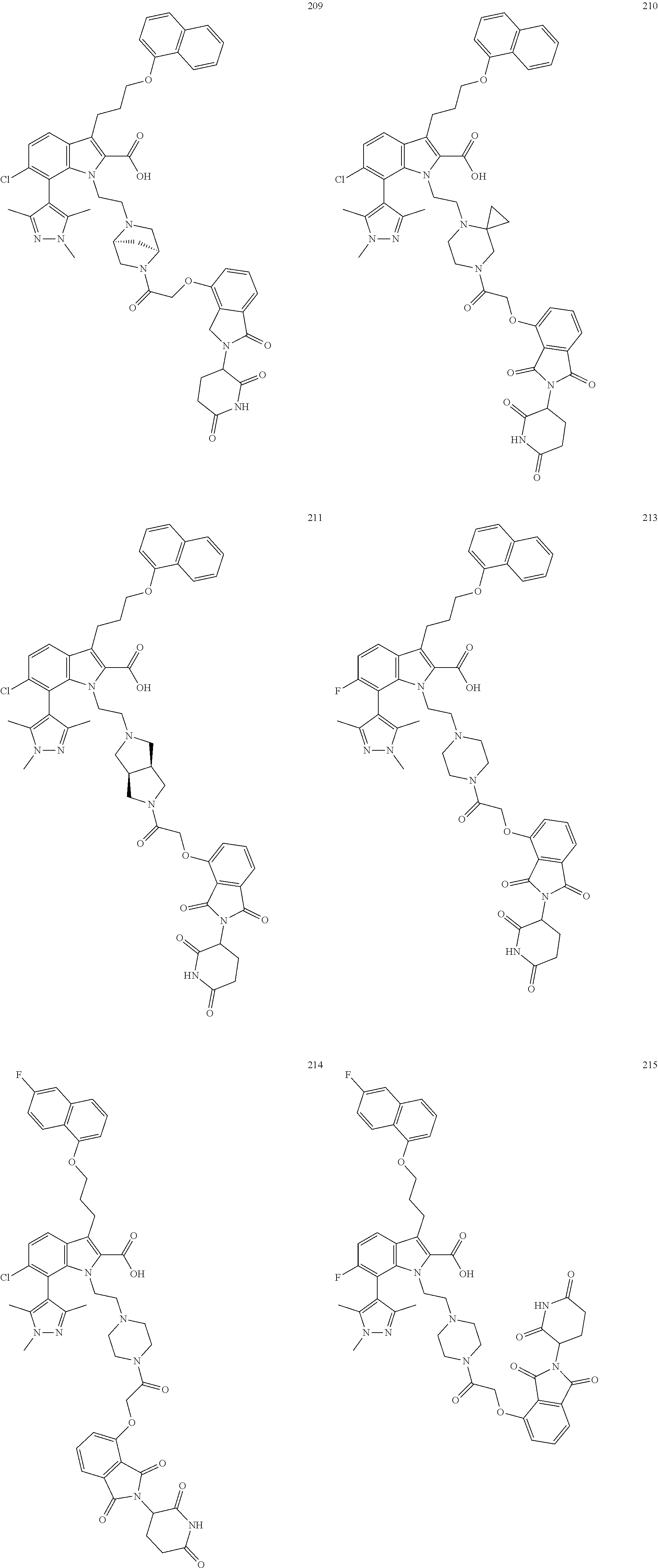

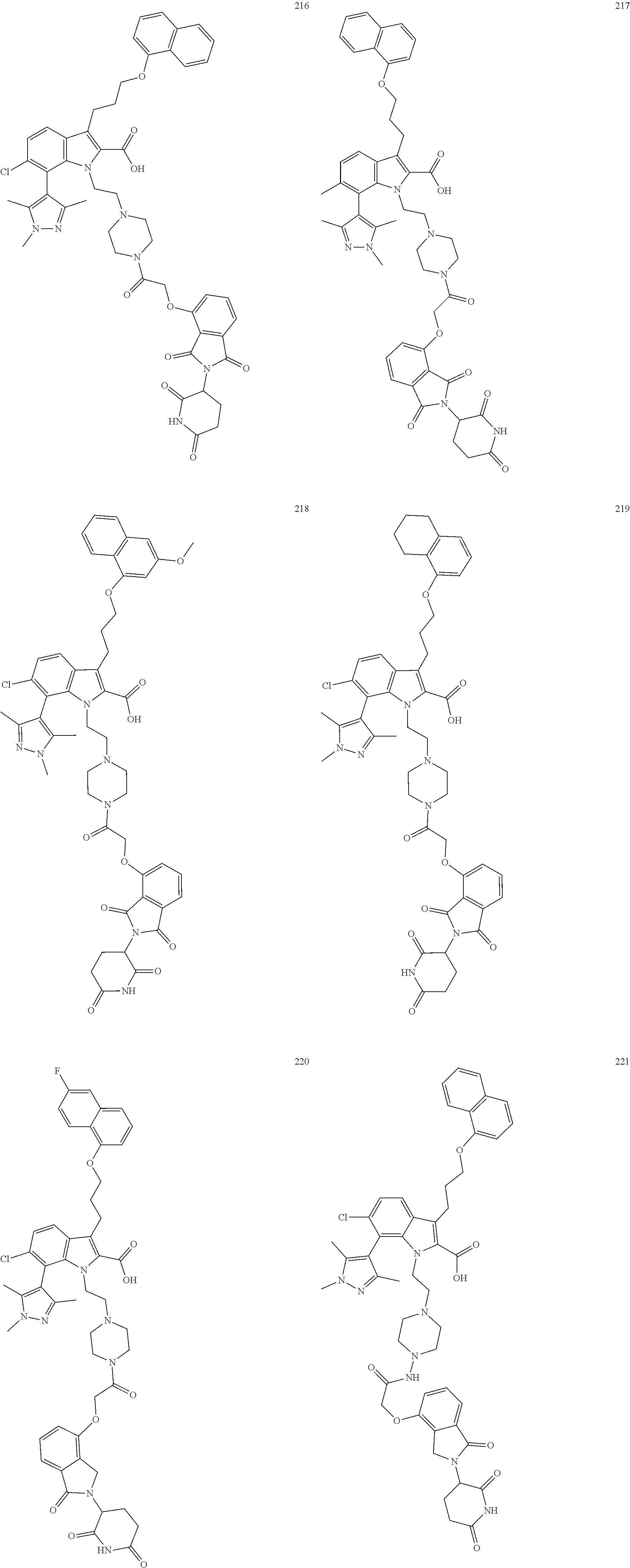

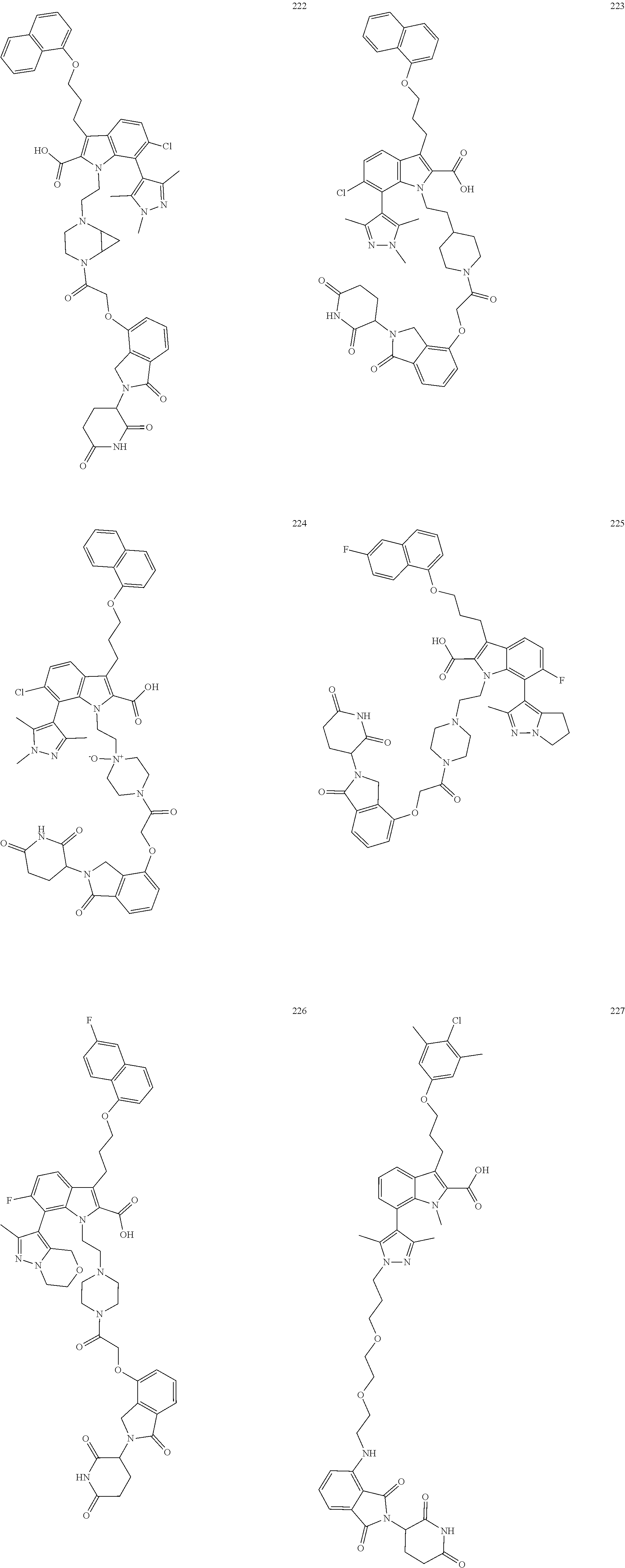

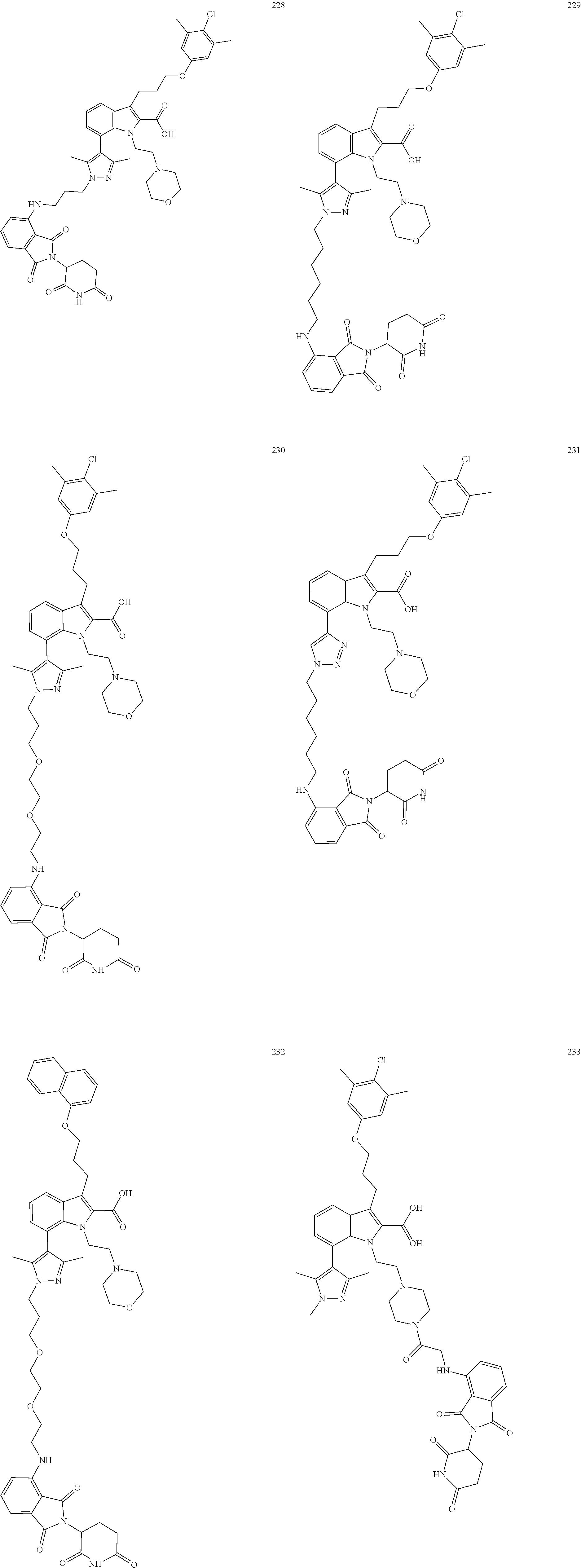

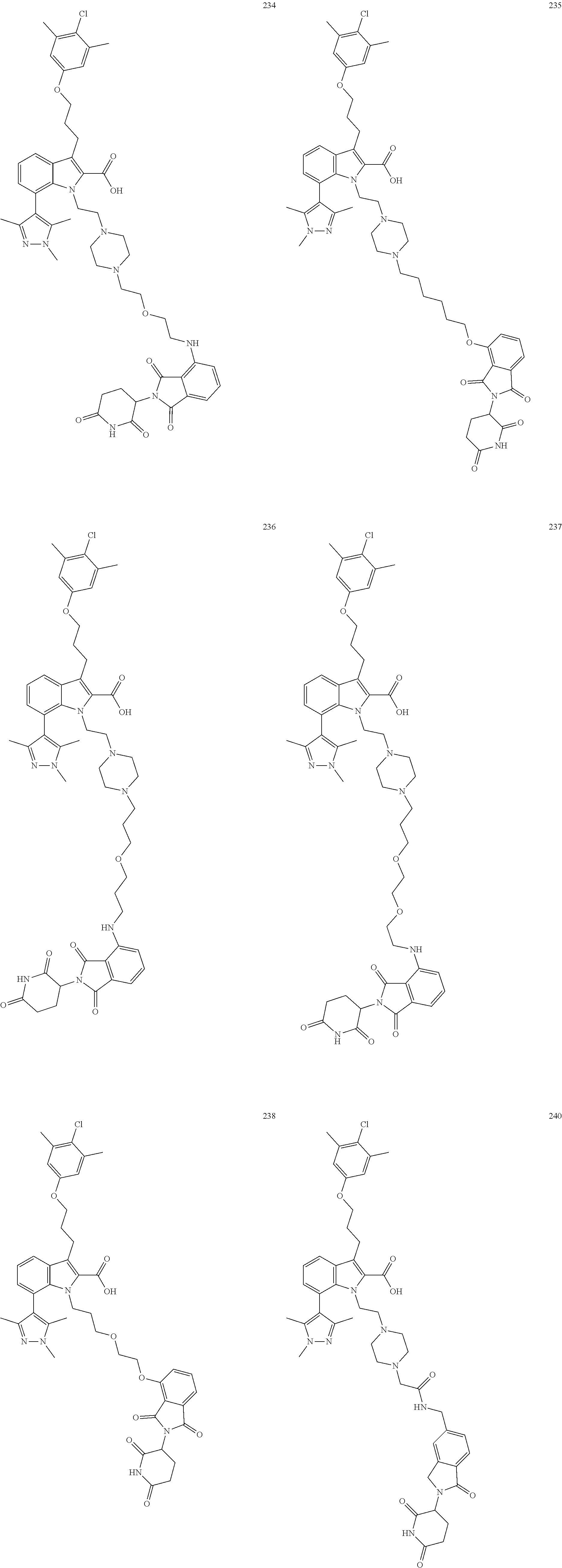

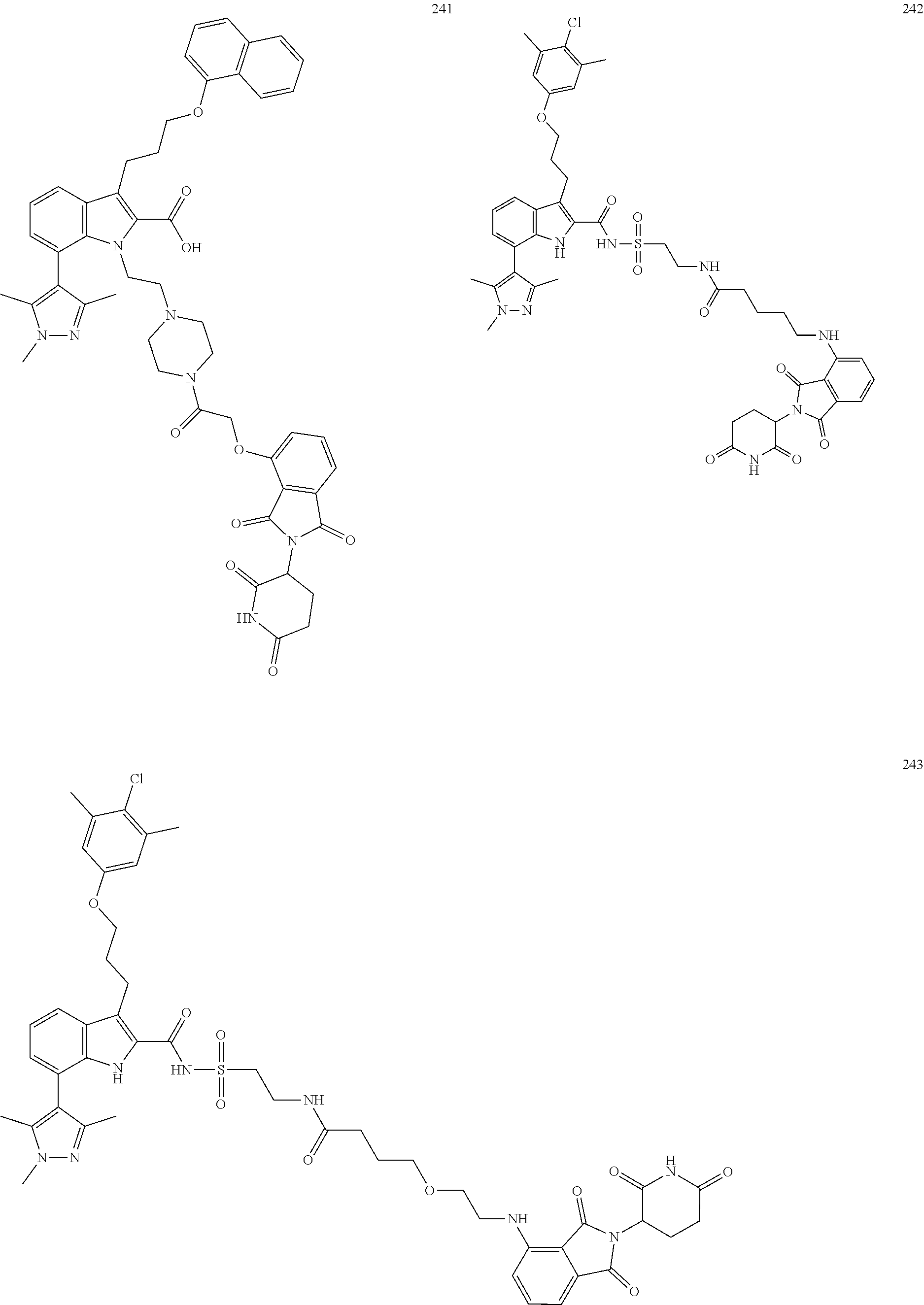 [SMILES: Cc1nn(C)c(C)c1-c1cccc2c(CCCOc3cccc4ccccc34)c(C(=O)O)n(CCN3CCN(C(=O)COc4cccc5c4C(=O)N(C4CCC(=O)NC4=O)C5=O)CC3)c12 ; Cc1cc(OCCCc2c(C(=O)NS(=O)(=O)CCNC(=O)CCCCNc3cccc4c3C(=O)N(C3CCC(=O)NC3=O)C4=O)[nH]c3c(-c4c(C)nn(C)c4C)cccc23)cc(C)c1Cl ; Cc1cc(OCCCc2c(C(=O)NS(=O)(=O)CCNC(=O)CCCOCCNc3cccc4c3C(=O)N(C3CCC(=O)NC3=O)C4=O)[nH]c3c(-c4c(C)nn(C)c4C)cccc23)cc(C)c1Cl]

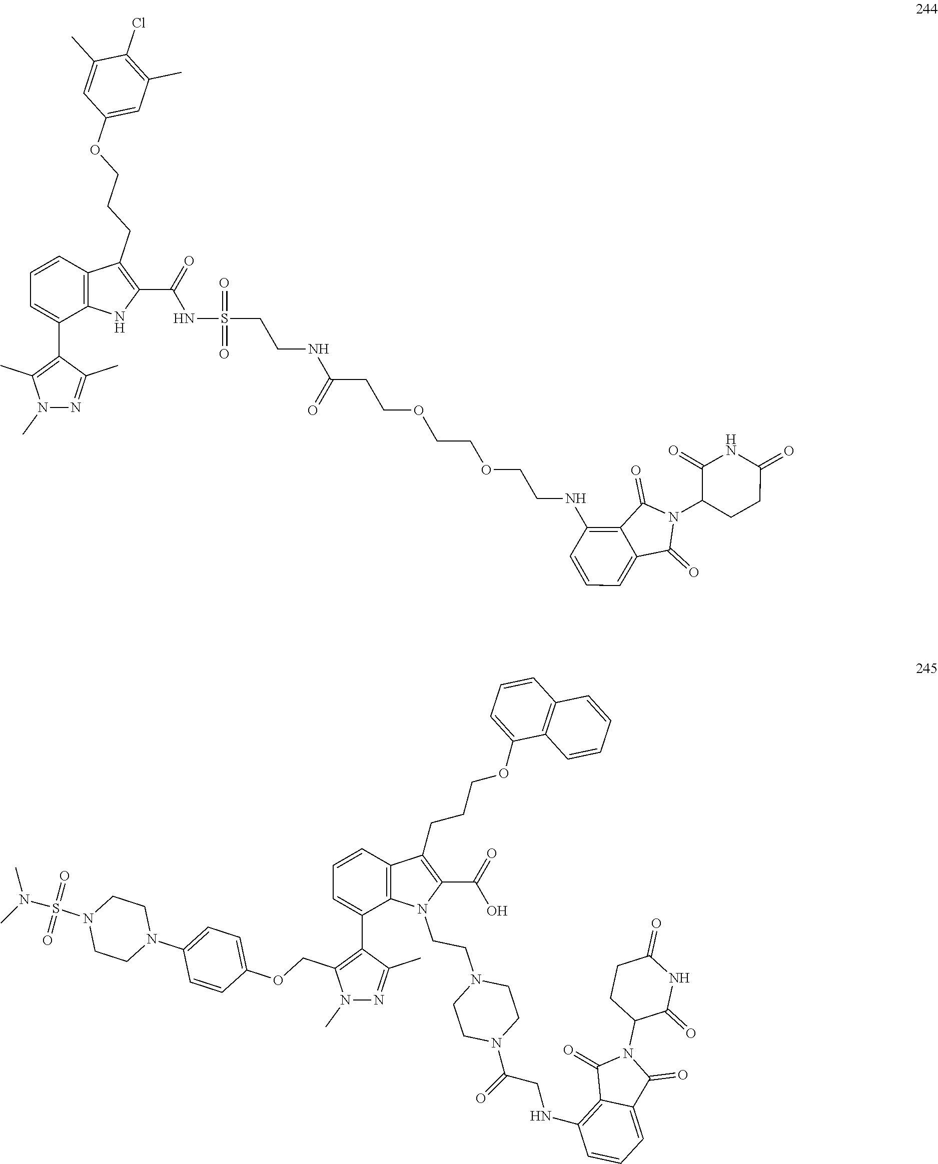 [SMILES: Cc1cc(OCCCc2c(C(=O)NS(=O)(=O)CCNC(=O)CCOCCOCCNc3cccc4c3C(=O)N(C3CCC(=O)NC3=O)C4=O)[nH]c3c(-c4c(C)nn(C)c4C)cccc23)cc(C)c1Cl ; Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(C(=O)CNc4cccc5c4C(=O)N(C4CCC(=O)NC4=O)C5=O)CC3)c12]

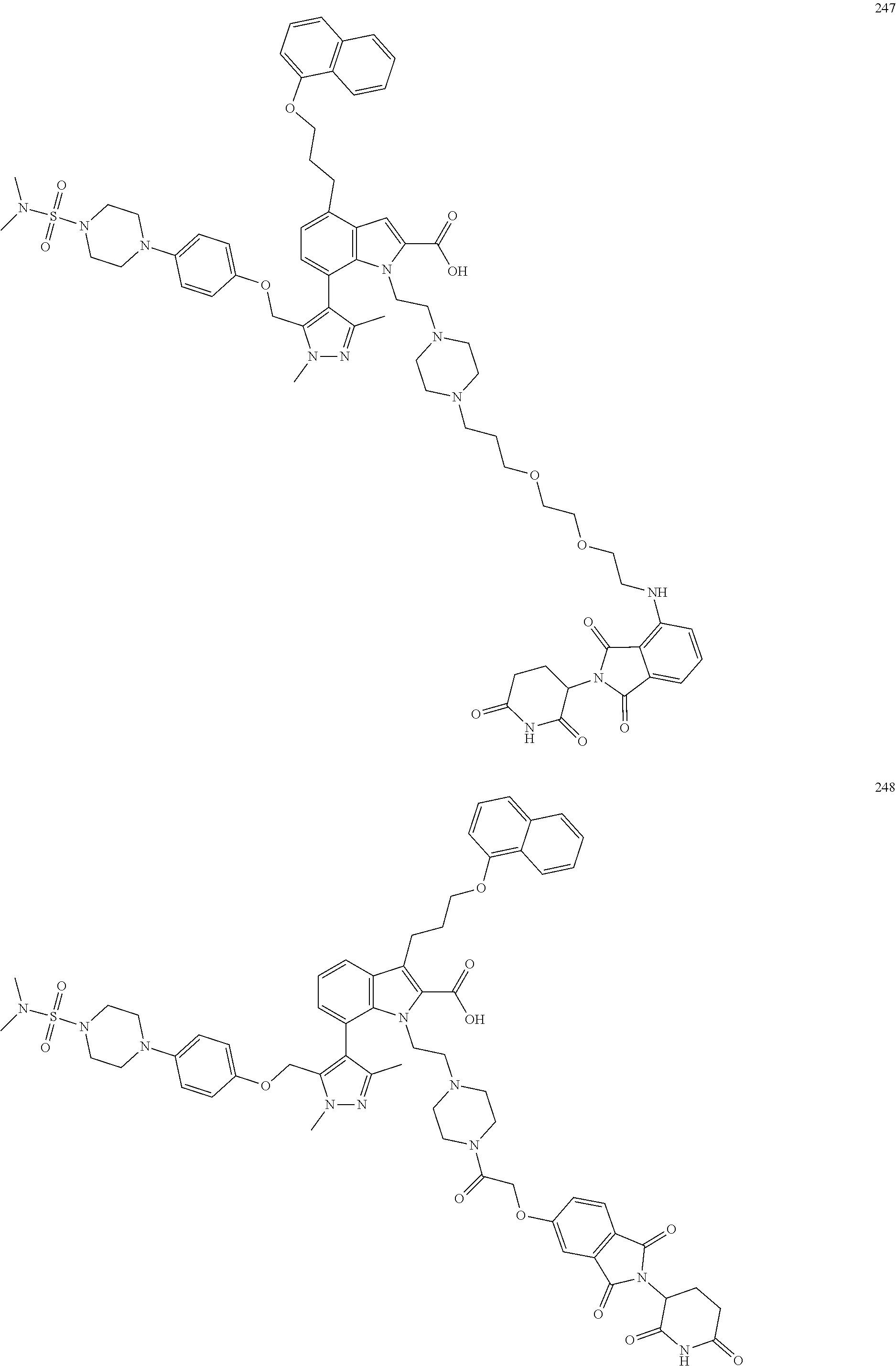 [SMILES: Cc1nn(C)c(COc2ccc(N3CCN(S(=O)(=O)N(C)C)CC3)cc2)c1-c1ccc(CCCOc2cccc3ccccc23)c2cc(C(=O)O)n(CCN3CCN(CCCOCCOCCNc4cccc5c4C(=O)N(C4CCC(=O)NC4=O)C5=O)CC3)c12 ; Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(C(=O)COc4ccc5c(c4)C(=O)N(C4CCC(=O)NC4=O)C5=O)CC3)c12]

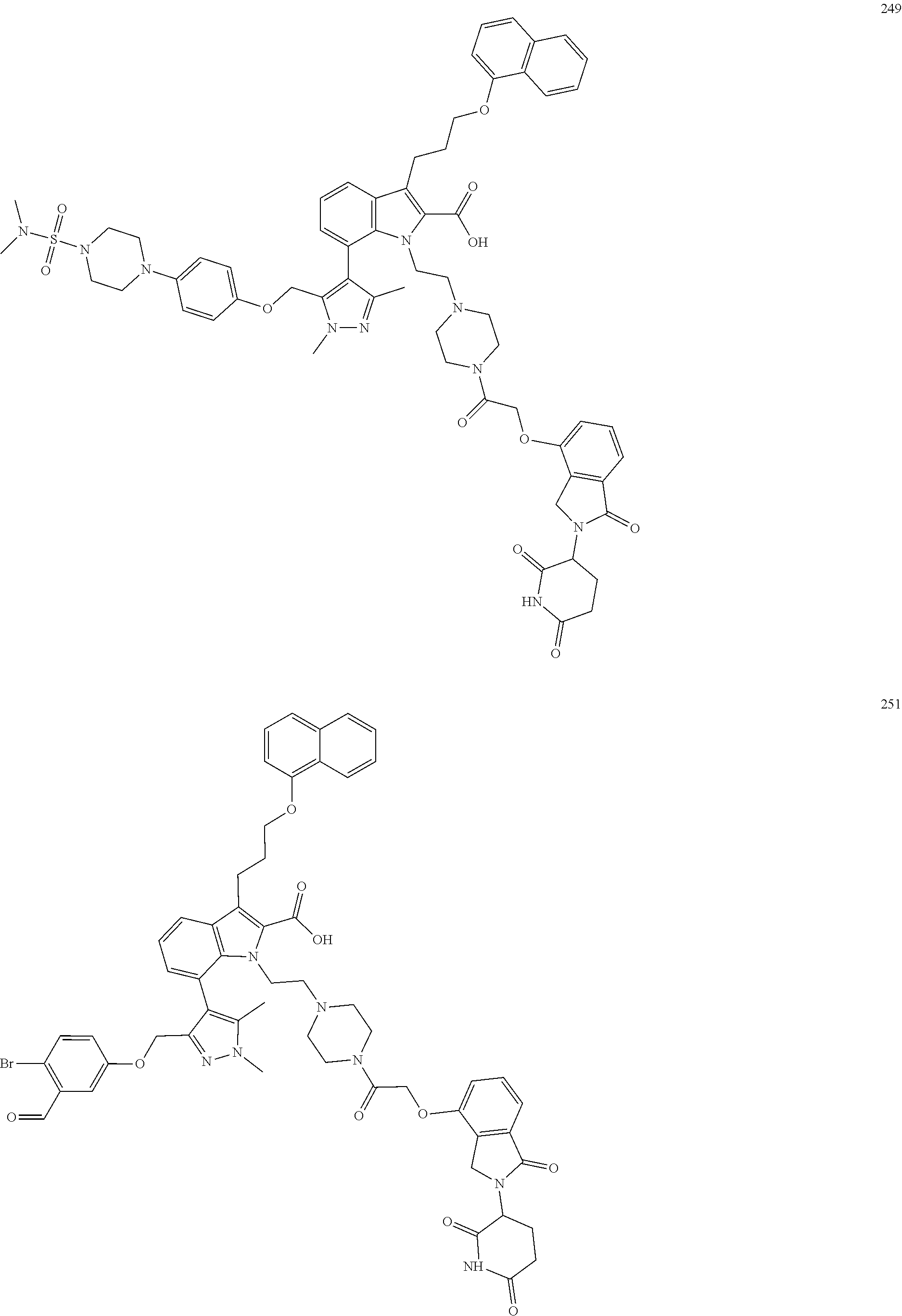 [SMILES: Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(C(=O)COc4cccc5c4CN(C4CCC(=O)NC4=O)C5=O)CC3)c12 ; Cc1c(-c2cccc3c(CCCOc4cccc5ccccc45)c(C(=O)O)n(CCN4CCN(C(=O)COc5cccc6c5CN(C5CCC(=O)NC5=O)C6=O)CC4)c23)c(COc2ccc(Br)c(C=O)c2)nn1C]

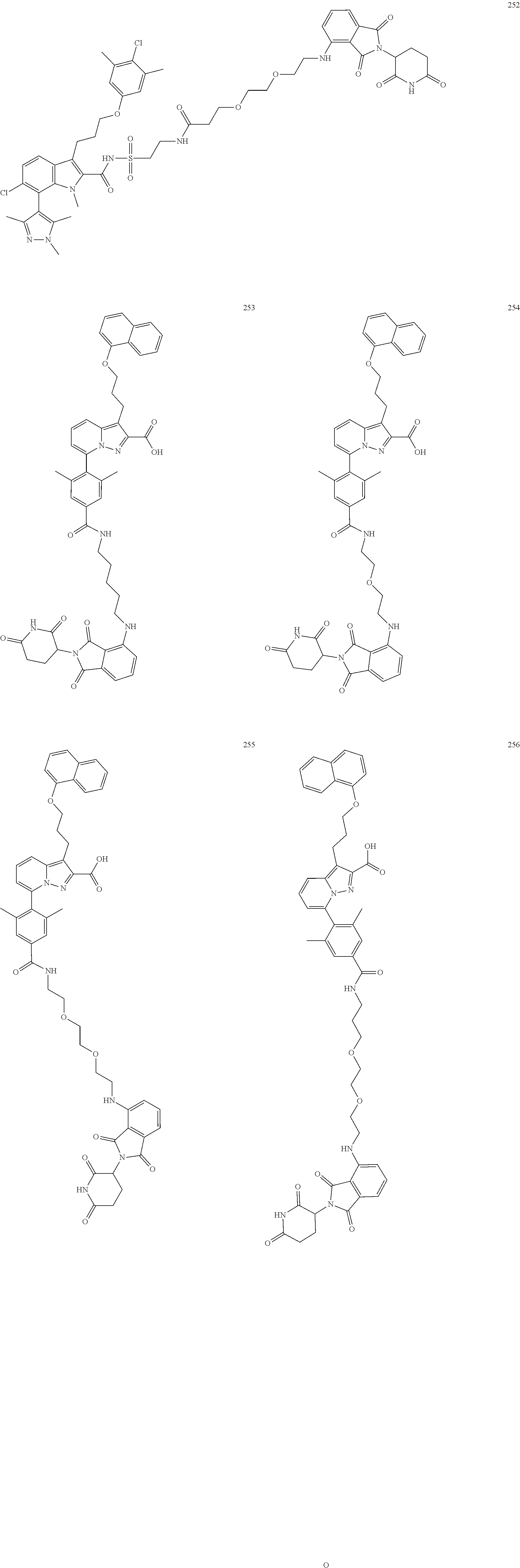

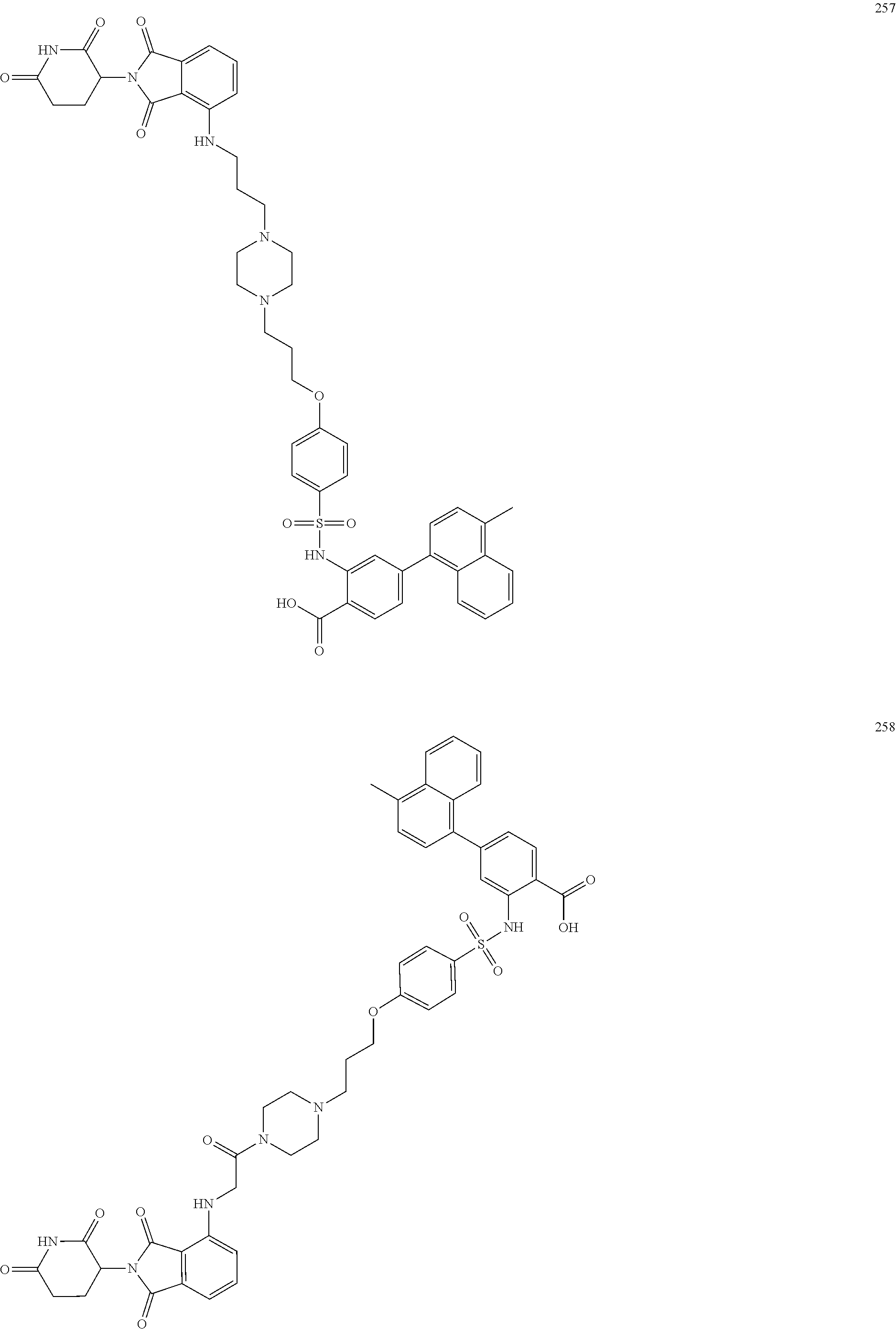 [SMILES: Cc1ccc(-c2ccc(C(=O)O)c(NS(=O)(=O)c3ccc(OCCCN4CCN(CCCNc5cccc6c5C(=O)N(C5CCC(=O)NC5=O)C6=O)CC4)cc3)c2)c2ccccc12 ; Cc1ccc(-c2ccc(C(=O)O)c(NS(=O)(=O)c3ccc(OCCCN4CCN(C(=O)CNc5cccc6c5C(=O)N(C5CCC(=O)NC5=O)C6=O)CC4)cc3)c2)c2ccccc12]

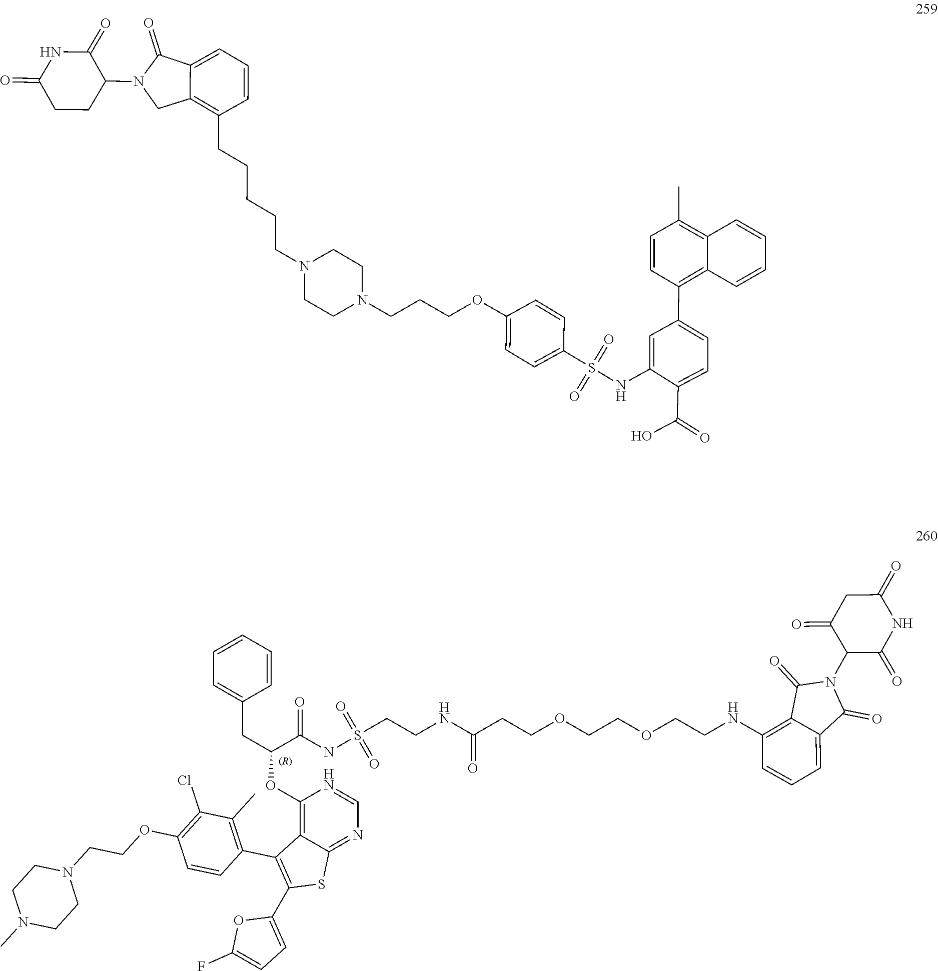

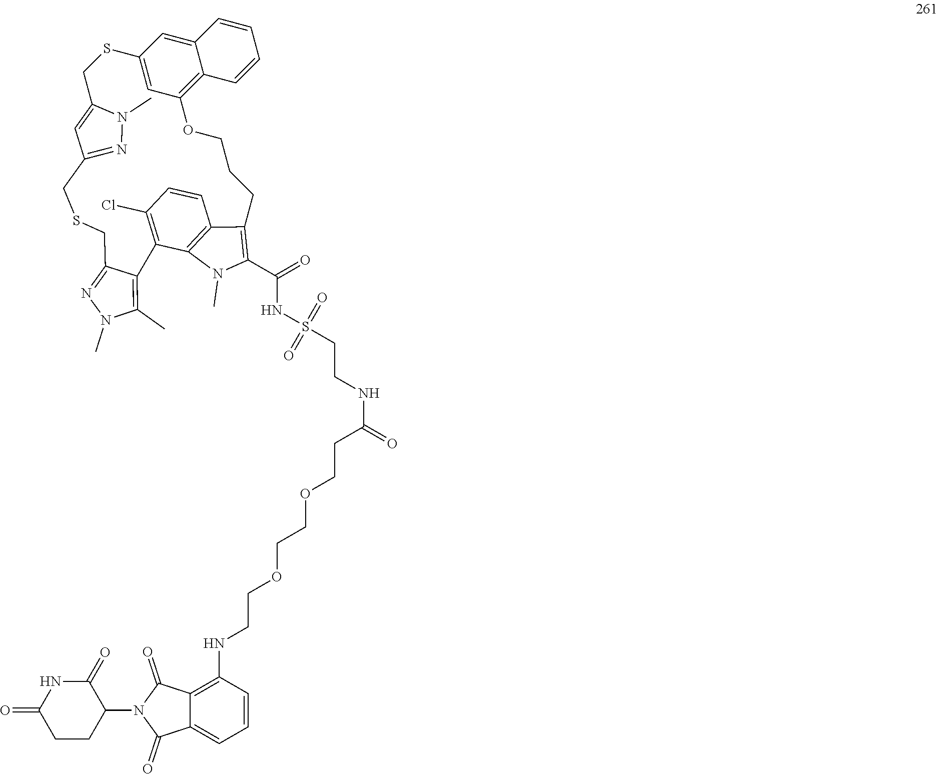 [SMILES: Cc1c2c(nn1C)CSCc1cc(n(C)n1)CSc1cc(c3ccccc3c1)OCCCc1c(C(=O)NS(=O)(=O)CCNC(=O)CCOCCOCCNc3cccc4c3C(=O)N(C3CCC(=O)NC3=O)C4=O)n(C)c3c-2c(Cl)ccc13]

45. The compound of claim 44, which is selected from:

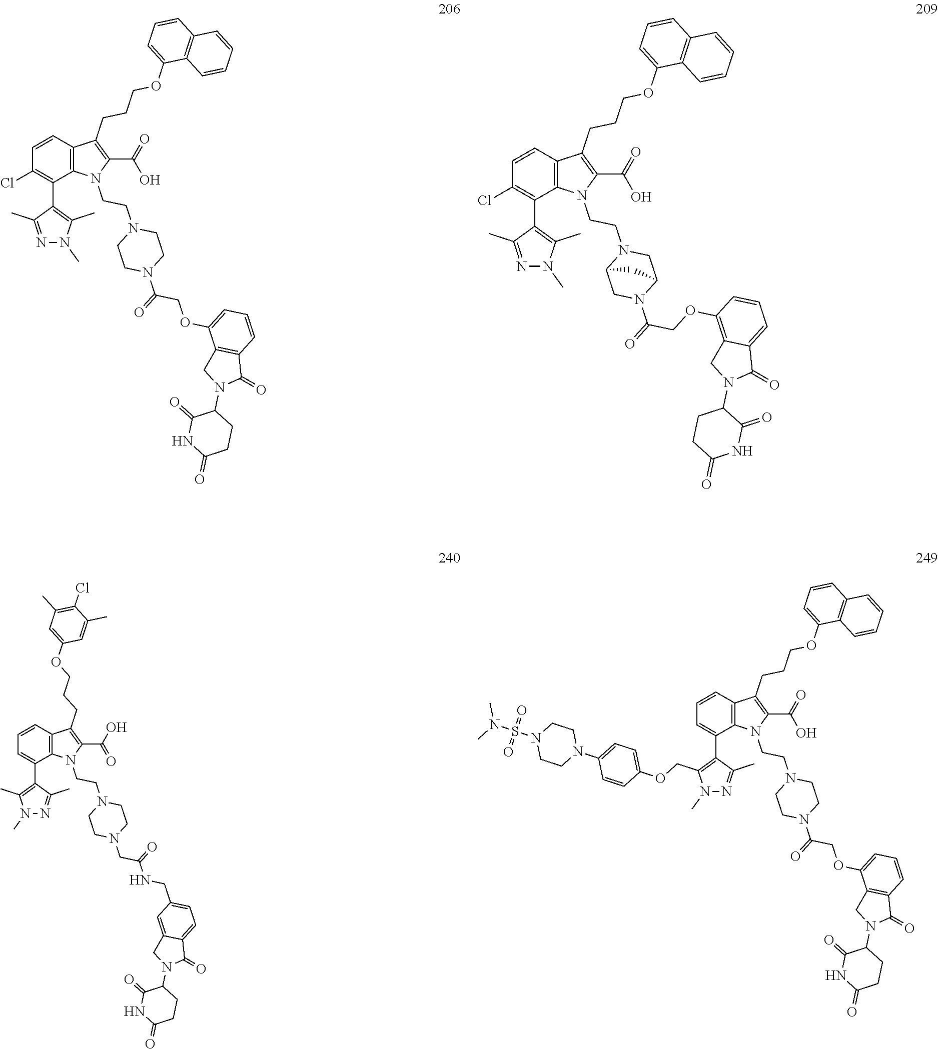 [SMILES: Cc1nn(C)c(C)c1-c1c(Cl)ccc2c(CCCOc3cccc4ccccc34)c(C(=O)O)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4ccccc34)c(C(=O)O)n(CCN3C[C@@H]4C[C@H]3CN4C(=O)COc3cccc4c3CN(C3CCC(=O)NC3=O)C4=O)c12 ; Cc1cc(OCCCc2c(C(=O)O)n(CCN3CCN(CC(=O)NCc4ccc5c(c4)CN(C4CCC(=O)NC4=O)C5=O)CC3)c3c(-c4c(C)nn(C)c4C)cccc23)cc(C)c1Cl ; Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(C(=O)COc4cccc5c4CN(C4CCC(=O)NC4=O)C5=O)CC3)c12]

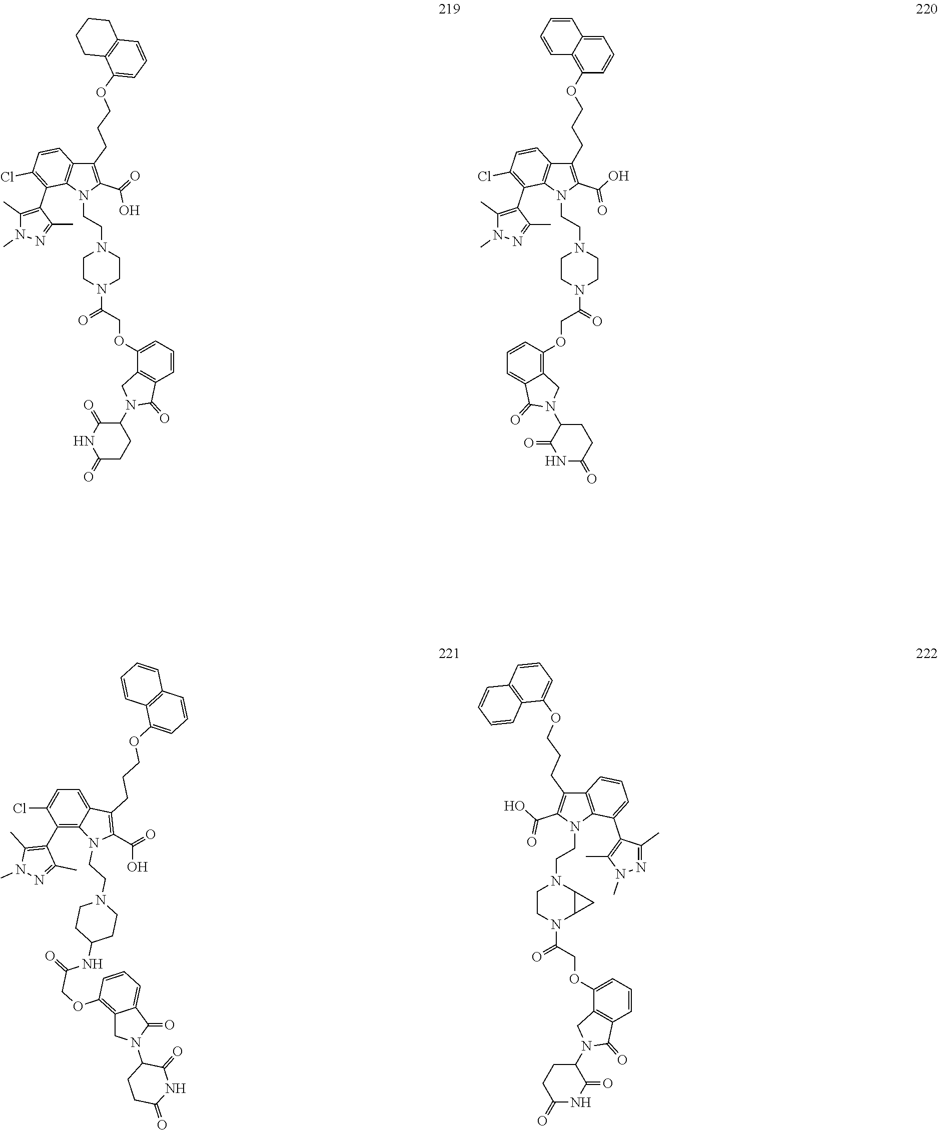 [SMILES: Cc1nn(C)c(C)c1-c1c(Cl)ccc2c(CCCOc3cccc4c3CCCC4)c(C(=O)O)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4ccccc34)c(C(=O)O)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4ccccc34)c(C(=O)O)n(CCN3CCC(NC(=O)COc4cccc5c4CN(C4CCC(=O)NC4=O)C5=O)CC3)c12 ; Cc1nn(C)c(C)c1-c1cccc2c(CCCOc3cccc4ccccc34)c(C(=O)O)n(CCN3CCN(C(=O)COc4cccc5c4CN(C4CCC(=O)NC4=O)C5=O)C4CC43)c12]

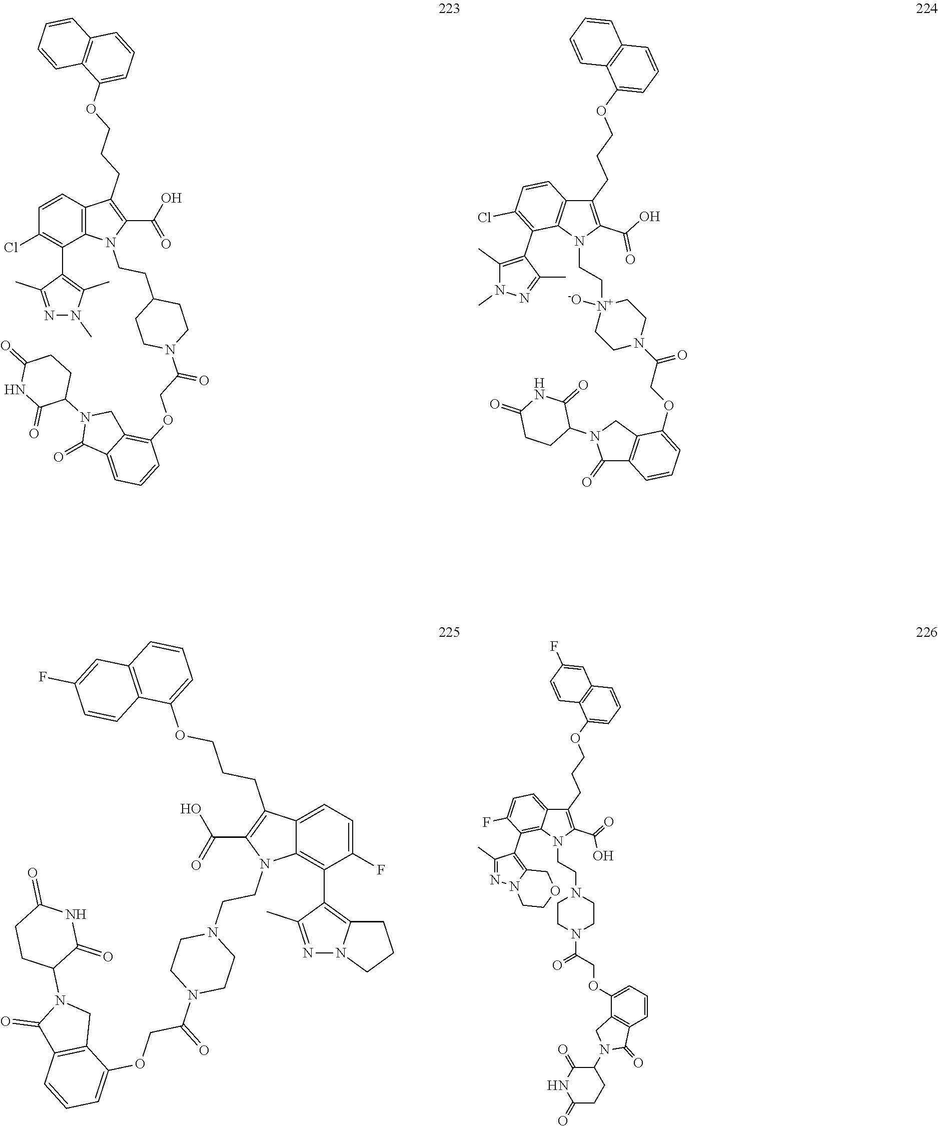

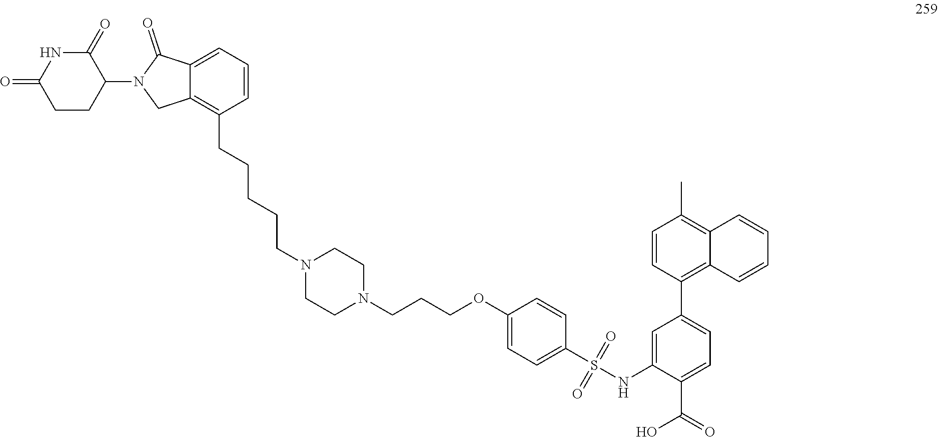 [SMILES: Cc1ccc(-c2ccc(C(=O)O)c(NS(=O)(=O)c3ccc(OCCCN4CCN(CCCCCc5cccc6c5CN(C5CCC(=O)NC5=O)C6=O)CC4)cc3)c2)c2ccccc12]

46. The compound of claim 44, which is selected from:

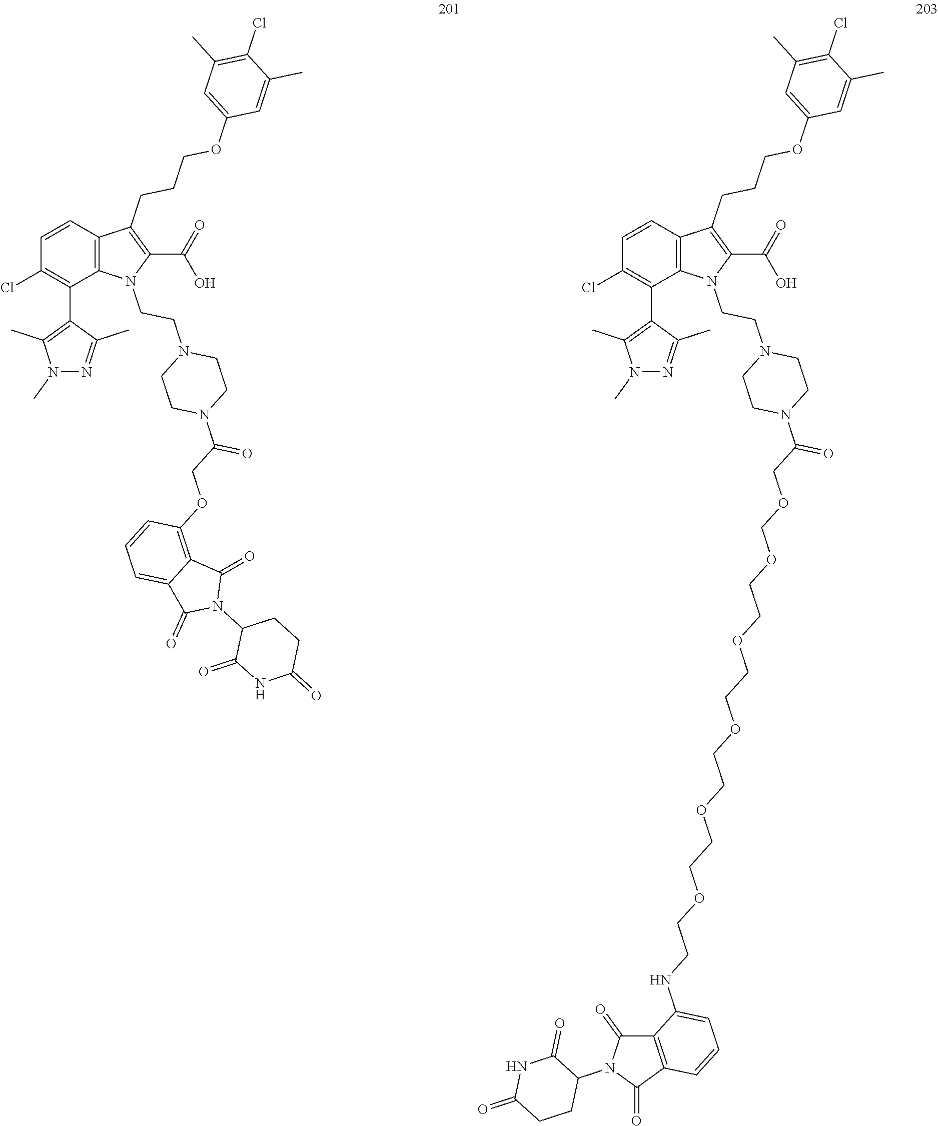 [SMILES: Cc1cc(OCCCc2c(C(=O)O)n(CCN3CCN(C(=O)COc4cccc5c4C(=O)N(C4CCC(=O)NC4=O)C5=O)CC3)c3c(-c4c(C)nn(C)c4C)c(Cl)ccc23)cc(C)c1Cl ; Cc1cc(OCCCc2c(C(=O)O)n(CCN3CCN(C(=O)COCOCCOCCOCCOCCOCCNc4cccc5c4C(=O)N(C4CCC(=O)NC4=O)C5=O)CC3)c3c(-c4c(C)nn(C)c4C)c(Cl)ccc23)cc(C)c1Cl]

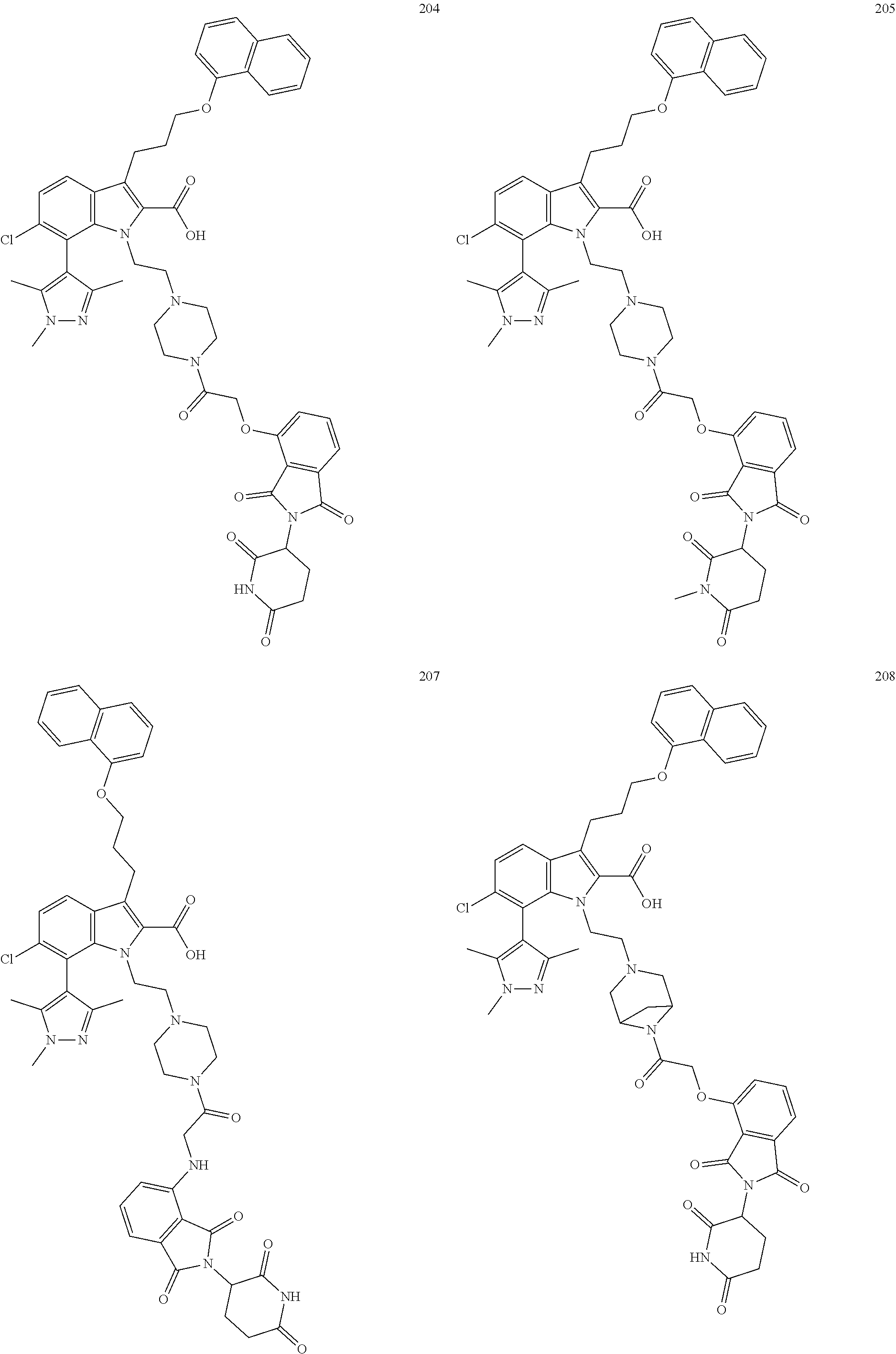 [SMILES: Cc1nn(C)c(C)c1-c1c(Cl)ccc2c(CCCOc3cccc4ccccc34)c(C(=O)O)n(CCN3CCN(C(=O)COc4cccc5c4C(=O)N(C4CCC(=O)NC4=O)C5=O)CC3)c12 ; Cc1nn(C)c(C)c1-c1c(Cl)ccc2c(CCCOc3cccc4ccccc34)c(C(=O)O)n(CCN3CCN(C(=O)COc4cccc5c4C(=O)N(C4CCC(=O)N(C)C4=O)C5=O)CC3)c12 ; Cc1nn(C)c(C)c1-c1c(Cl)ccc2c(CCCOc3cccc4ccccc34)c(C(=O)O)n(CCN3CCN(C(=O)CNc4cccc5c4C(=O)N(C4CCC(=O)NC4=O)C5=O)CC3)c12 ; Cc1nn(C)c(C)c1-c1c(Cl)ccc2c(CCCOc3cccc4ccccc34)c(C(=O)O)n(CCN3CC4CC(C3)N4C(=O)COc3cccc4c3C(=O)N(C3CCC(=O)NC3=O)C4=O)c12]

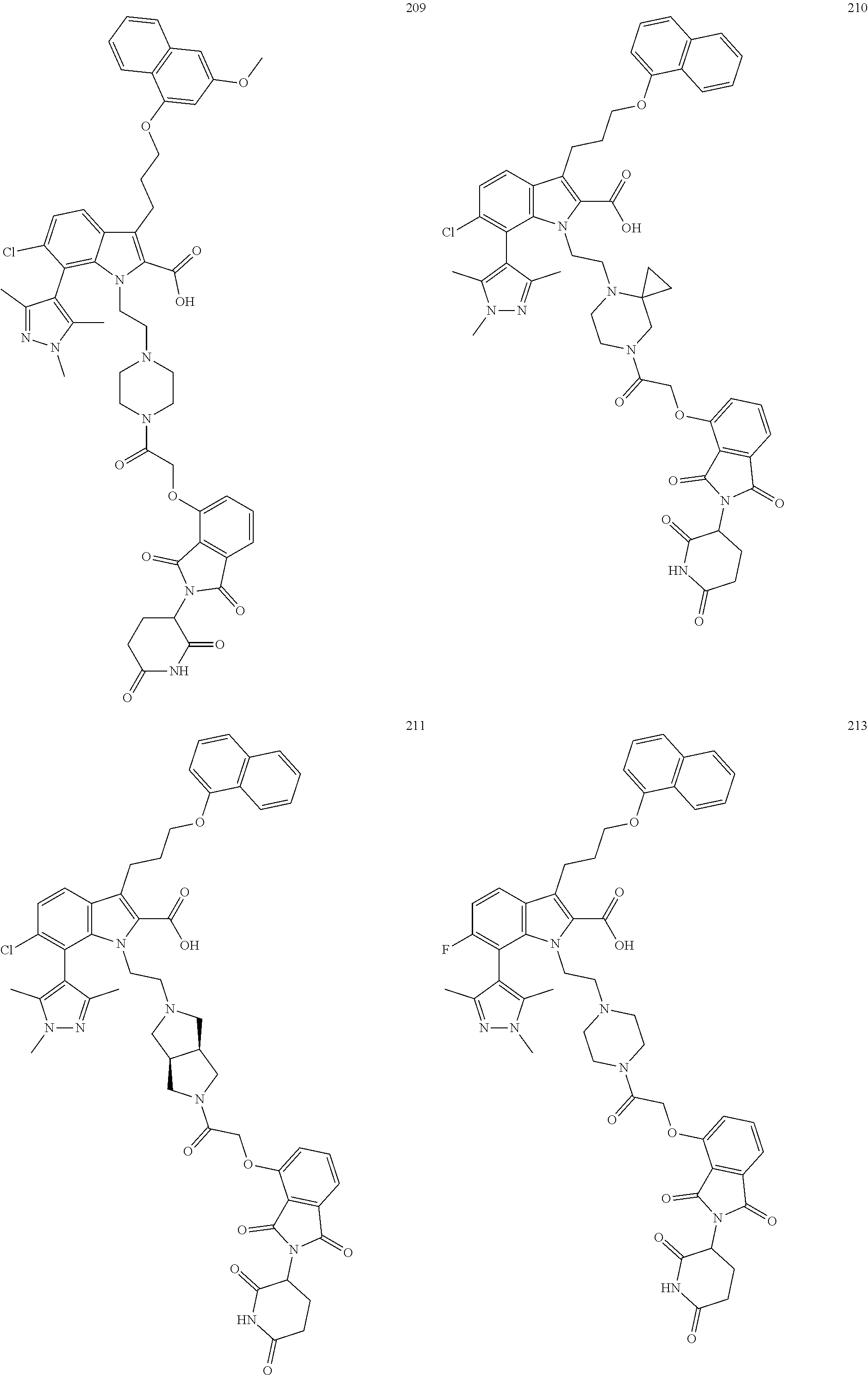

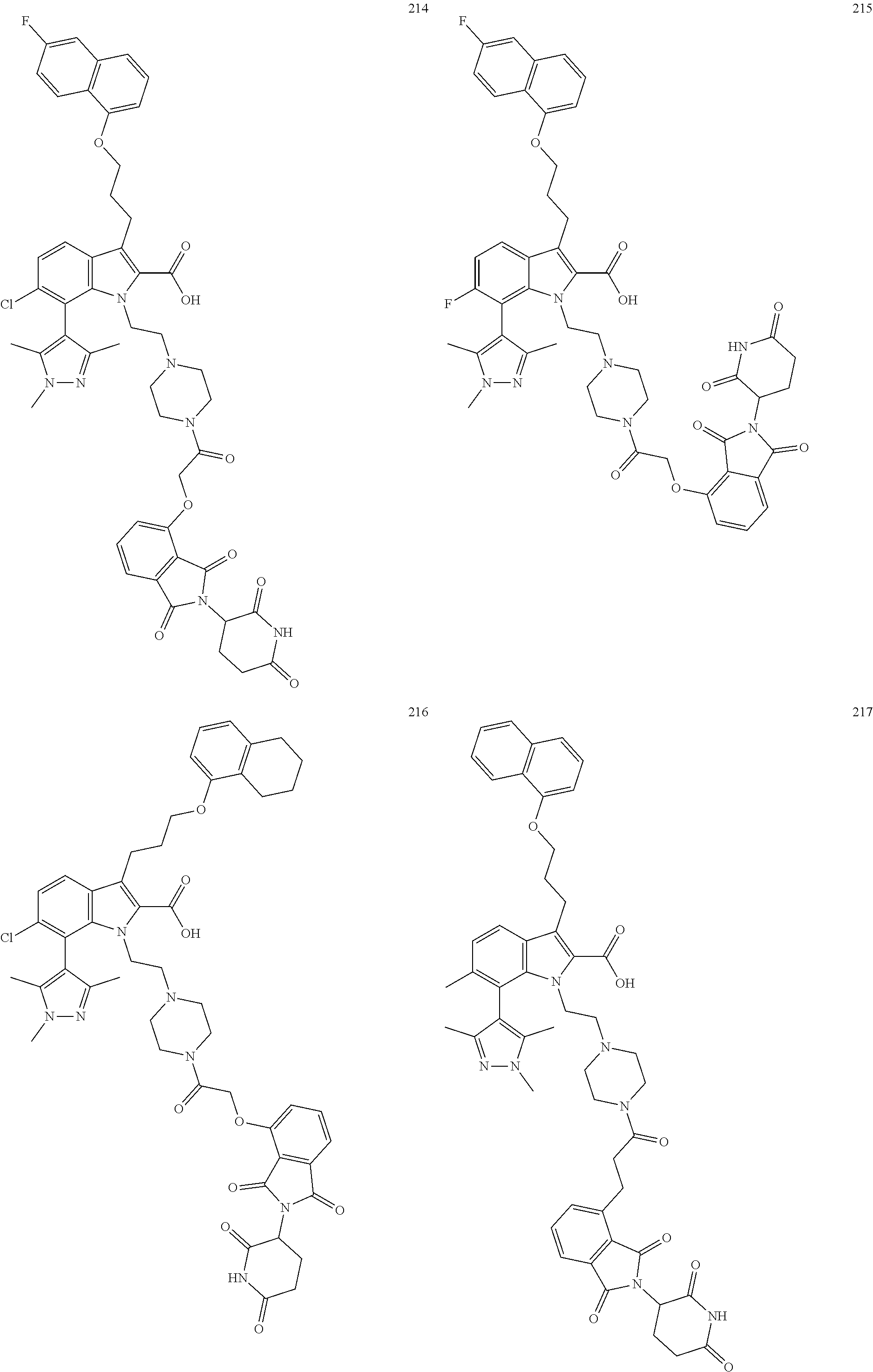 [SMILES: Cc1nn(C)c(C)c1-c1c(Cl)ccc2c(CCCOc3cccc4cc(F)ccc34)c(C(=O)O)n(CCN3CCN(C(=O)COc4cccc5c4C(=O)N(C4CCC(=O)NC4=O)C5=O)CC3)c12 ; Cc1nn(C)c(C)c1-c1c(F)ccc2c(CCCOc3cccc4cc(F)ccc34)c(C(=O)O)n(CCN3CCN(C(=O)COc4cccc5c4C(=O)N(C4CCC(=O)NC4=O)C5=O)CC3)c12 ; Cc1nn(C)c(C)c1-c1c(Cl)ccc2c(CCCOc3cccc4c3CCCC4)c(C(=O)O)n(CCN3CCN(C(=O)COc4cccc5c4C(=O)N(C4CCC(=O)NC4=O)C5=O)CC3)c12 ; Cc1ccc2c(CCCOc3cccc4ccccc34)c(C(=O)O)n(CCN3CCN(C(=O)CCc4cccc5c4C(=O)N(C4CCC(=O)NC4=O)C5=O)CC3)c2c1-c1c(C)nn(C)c1C]

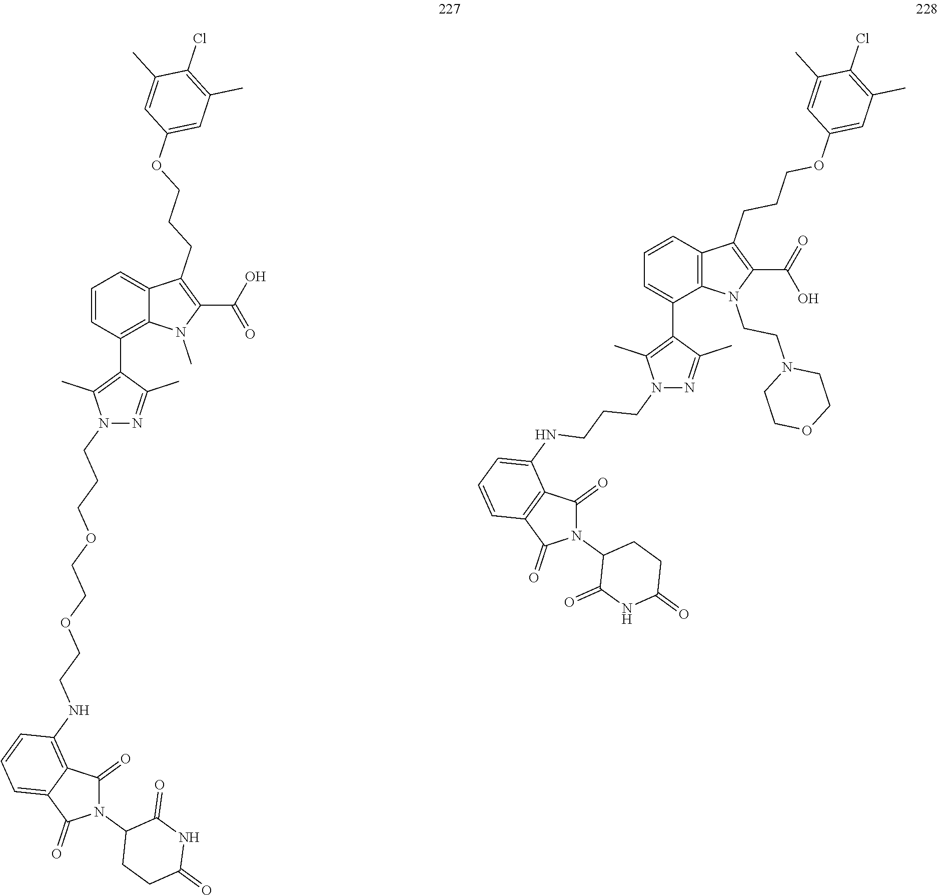 [SMILES: Cc1cc(OCCCc2c(C(=O)O)n(C)c3c(-c4c(C)nn(CCCOCCOCCNc5cccc6c5C(=O)N(C5CCC(=O)NC5=O)C6=O)c4C)cccc23)cc(C)c1Cl ; Cc1cc(OCCCc2c(C(=O)O)n(CCN3CCOCC3)c3c(-c4c(C)nn(CCCNc5cccc6c5C(=O)N(C5CCC(=O)NC5=O)C6=O)c4C)cccc23)cc(C)c1Cl]

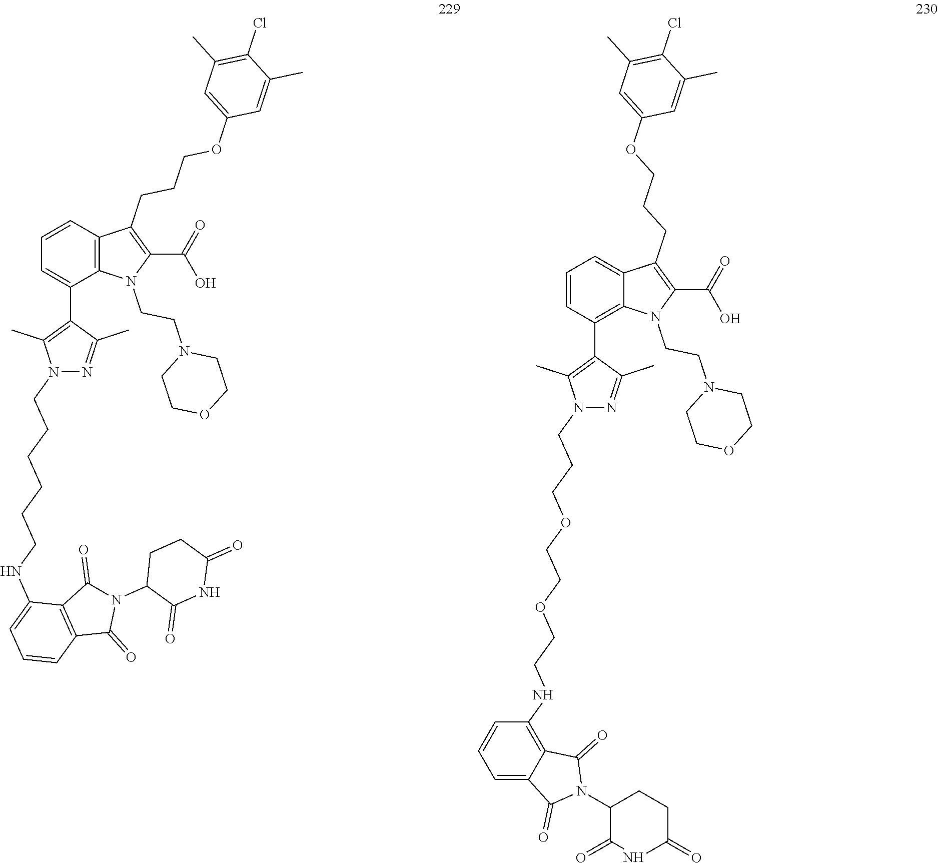 [SMILES: Cc1cc(OCCCc2c(C(=O)O)n(CCN3CCOCC3)c3c(-c4c(C)nn(CCCCCCNc5cccc6c5C(=O)N(C5CCC(=O)NC5=O)C6=O)c4C)cccc23)cc(C)c1Cl ; Cc1cc(OCCCc2c(C(=O)O)n(CCN3CCOCC3)c3c(-c4c(C)nn(CCCOCCOCCNc5cccc6c5C(=O)N(C5CCC(=O)NC5=O)C6=O)c4C)cccc23)cc(C)c1Cl]

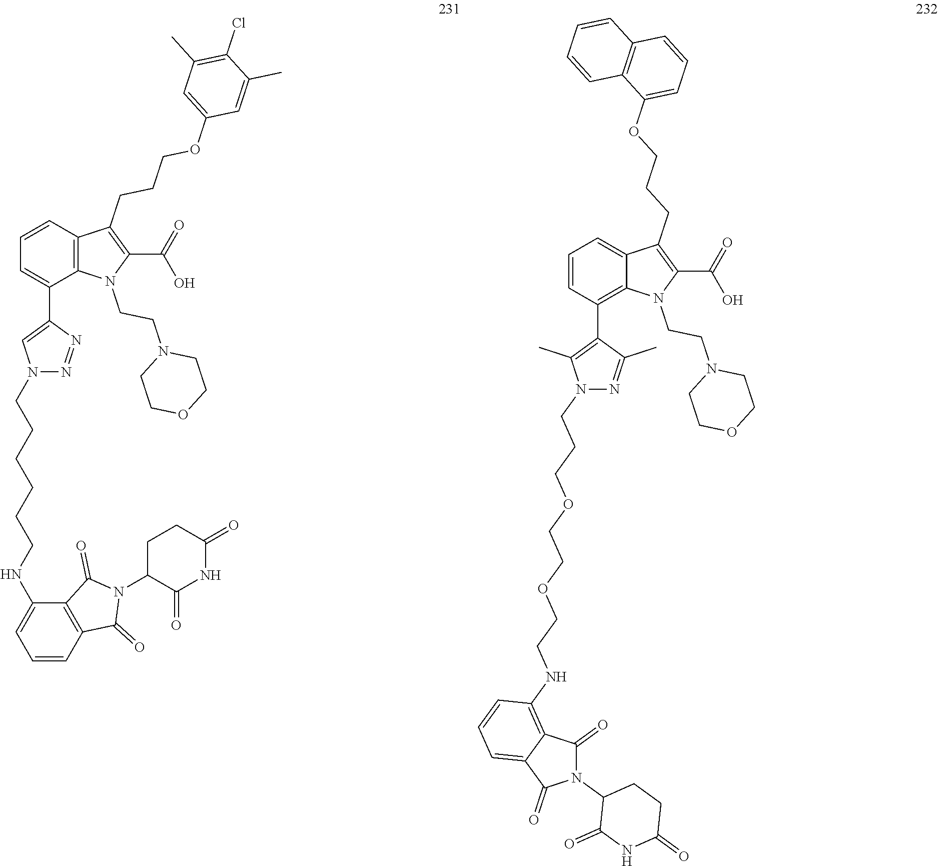 [SMILES: Cc1cc(OCCCc2c(C(=O)O)n(CCN3CCOCC3)c3c(-c4cn(CCCCCCNc5cccc6c5C(=O)N(C5CCC(=O)NC5=O)C6=O)nn4)cccc23)cc(C)c1Cl ; Cc1nn(CCCOCCOCCNc2cccc3c2C(=O)N(C2CCC(=O)NC2=O)C3=O)c(C)c1-c1cccc2c(CCCOc3cccc4ccccc34)c(C(=O)O)n(CCN3CCOCC3)c12]

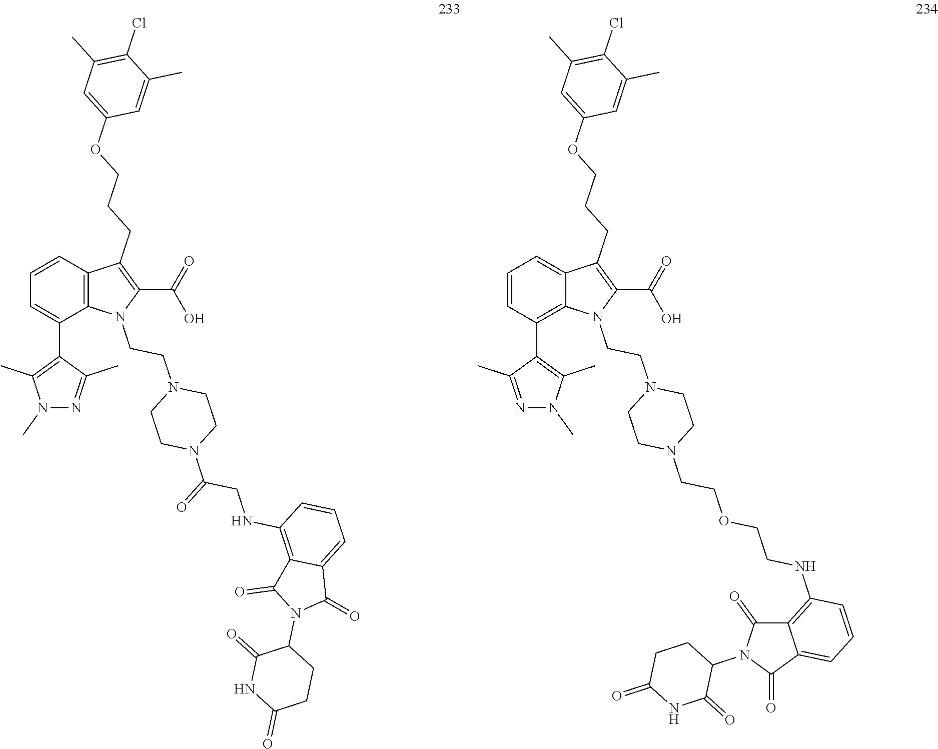 [SMILES: Cc1cc(OCCCc2c(C(=O)O)n(CCN3CCN(C(=O)CNc4cccc5c4C(=O)N(C4CCC(=O)NC4=O)C5=O)CC3)c3c(-c4c(C)nn(C)c4C)cccc23)cc(C)c1Cl ; Cc1cc(OCCCc2c(C(=O)O)n(CCN3CCN(CCOCCNc4cccc5c4C(=O)N(C4CCC(=O)NC4=O)C5=O)CC3)c3c(-c4c(C)nn(C)c4C)cccc23)cc(C)c1Cl]

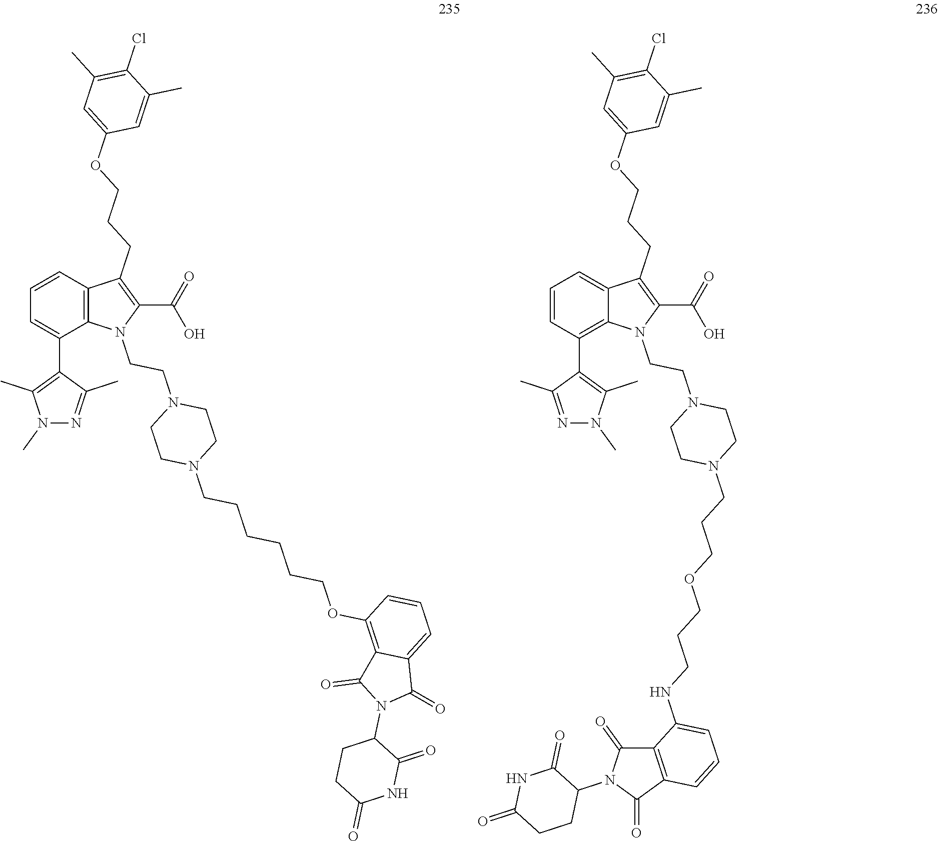 [SMILES: Cc1cc(OCCCc2c(C(=O)O)n(CCN3CCN(CCCCCCOc4cccc5c4C(=O)N(C4CCC(=O)NC4=O)C5=O)CC3)c3c(-c4c(C)nn(C)c4C)cccc23)cc(C)c1Cl ; Cc1cc(OCCCc2c(C(=O)O)n(CCN3CCN(CCCOCCCNc4cccc5c4C(=O)N(C4CCC(=O)NC4=O)C5=O)CC3)c3c(-c4c(C)nn(C)c4C)cccc23)cc(C)c1Cl]

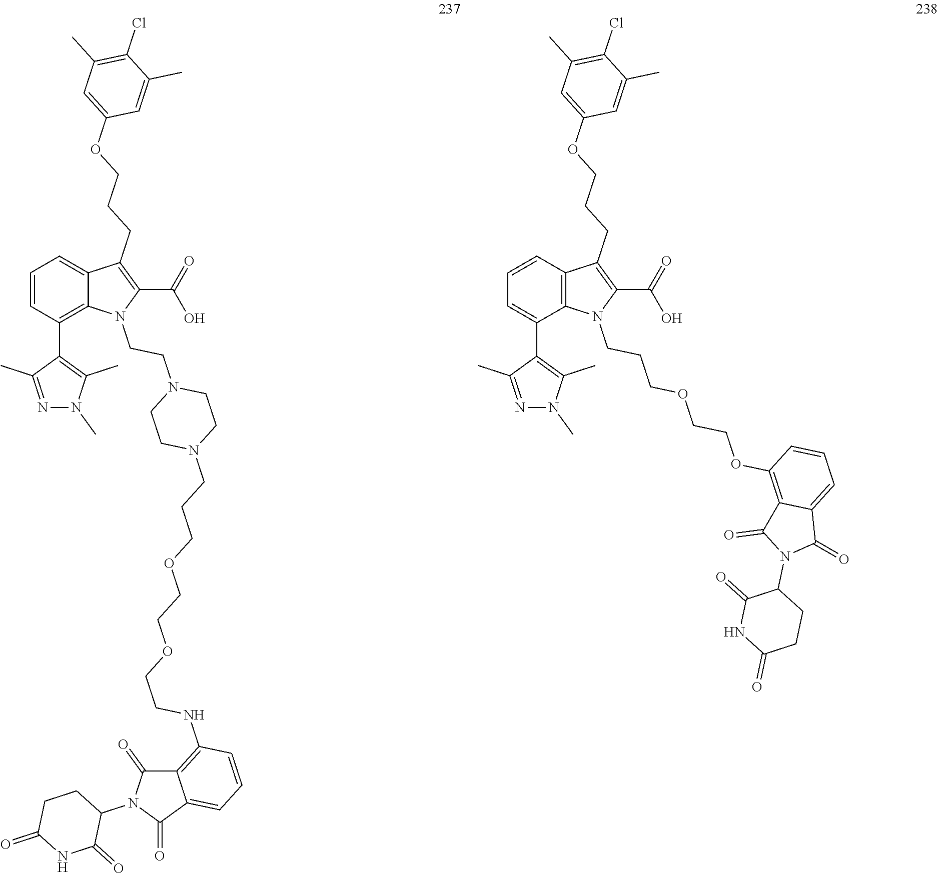 [SMILES: Cc1cc(OCCCc2c(C(=O)O)n(CCN3CCN(CCCOCCOCCNc4cccc5c4C(=O)N(C4CCC(=O)NC4=O)C5=O)CC3)c3c(-c4c(C)nn(C)c4C)cccc23)cc(C)c1Cl ; Cc1cc(OCCCc2c(C(=O)O)n(CCCOCCOc3cccc4c3C(=O)N(C3CCC(=O)NC3=O)C4=O)c3c(-c4c(C)nn(C)c4C)cccc23)cc(C)c1Cl]

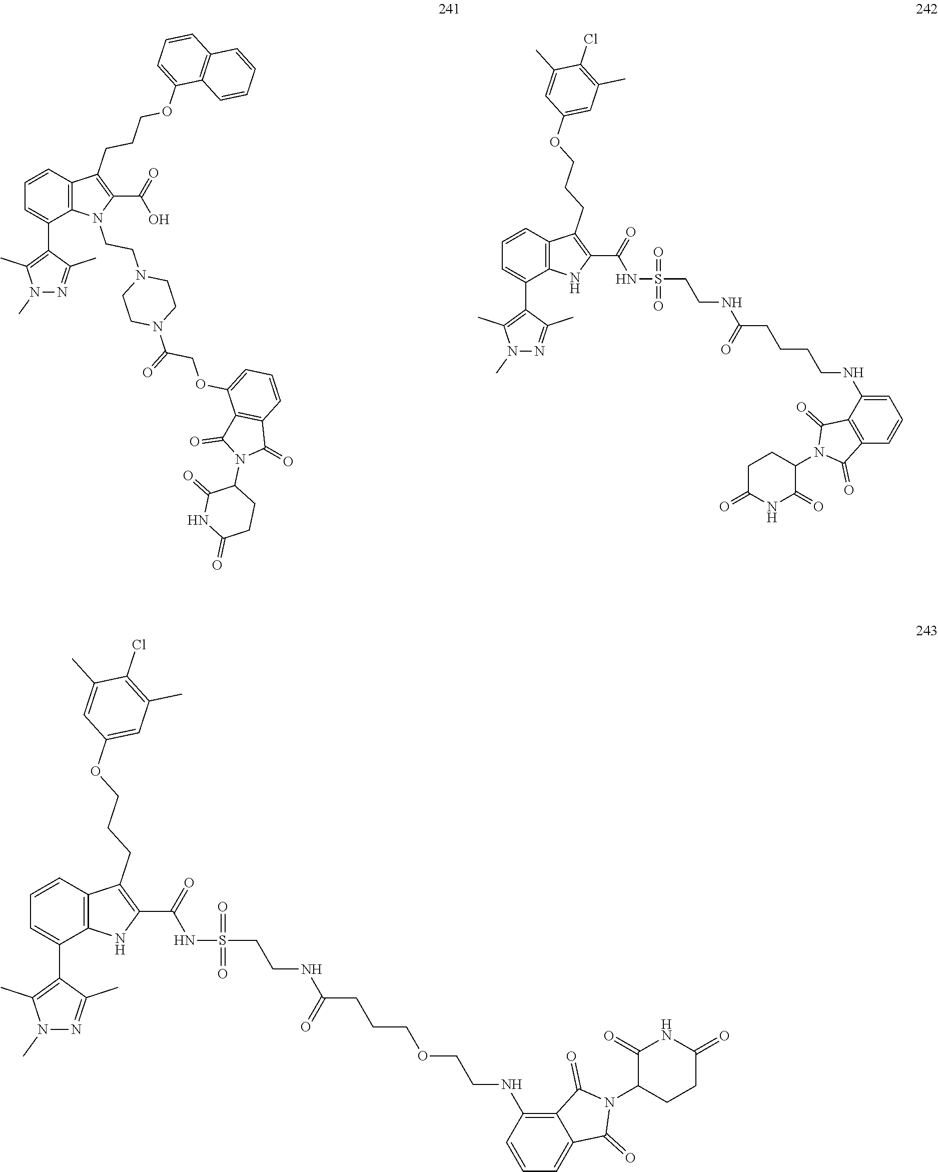 [SMILES: Cc1nn(C)c(C)c1-c1cccc2c(CCCOc3cccc4ccccc34)c(C(=O)O)n(CCN3CCN(C(=O)COc4cccc5c4C(=O)N(C4CCC(=O)NC4=O)C5=O)CC3)c12 ; Cc1cc(OCCCc2c(C(=O)NS(=O)(=O)CCNC(=O)CCCCNc3cccc4c3C(=O)N(C3CCC(=O)NC3=O)C4=O)[nH]c3c(-c4c(C)nn(C)c4C)cccc23)cc(C)c1Cl ; Cc1cc(OCCCc2c(C(=O)NS(=O)(=O)CCNC(=O)CCCOCCNc3cccc4c3C(=O)N(C3CCC(=O)NC3=O)C4=O)[nH]c3c(-c4c(C)nn(C)c4C)cccc23)cc(C)c1Cl]

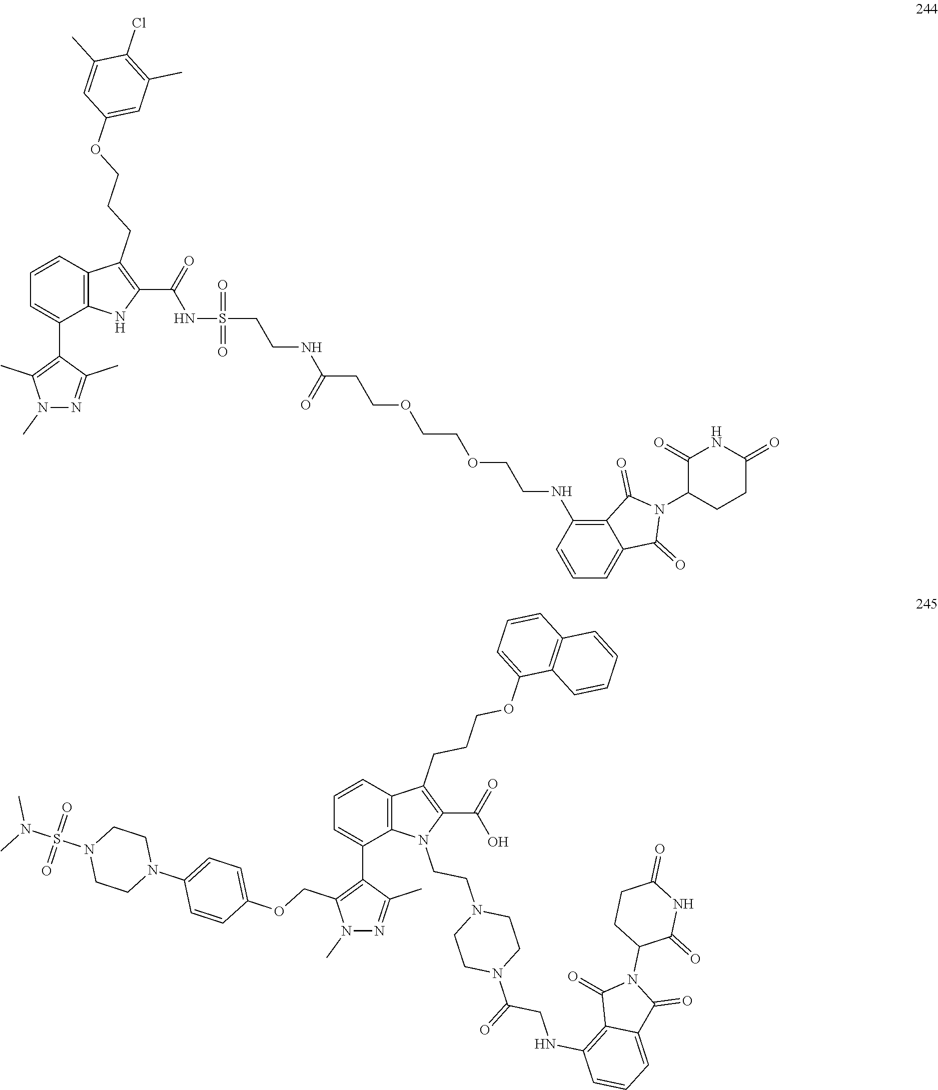 [SMILES: Cc1cc(OCCCc2c(C(=O)NS(=O)(=O)CCNC(=O)CCOCCOCCNc3cccc4c3C(=O)N(C3CCC(=O)NC3=O)C4=O)[nH]c3c(-c4c(C)nn(C)c4C)cccc23)cc(C)c1Cl ; Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(C(=O)CNc4cccc5c4C(=O)N(C4CCC(=O)NC4=O)C5=O)CC3)c12]

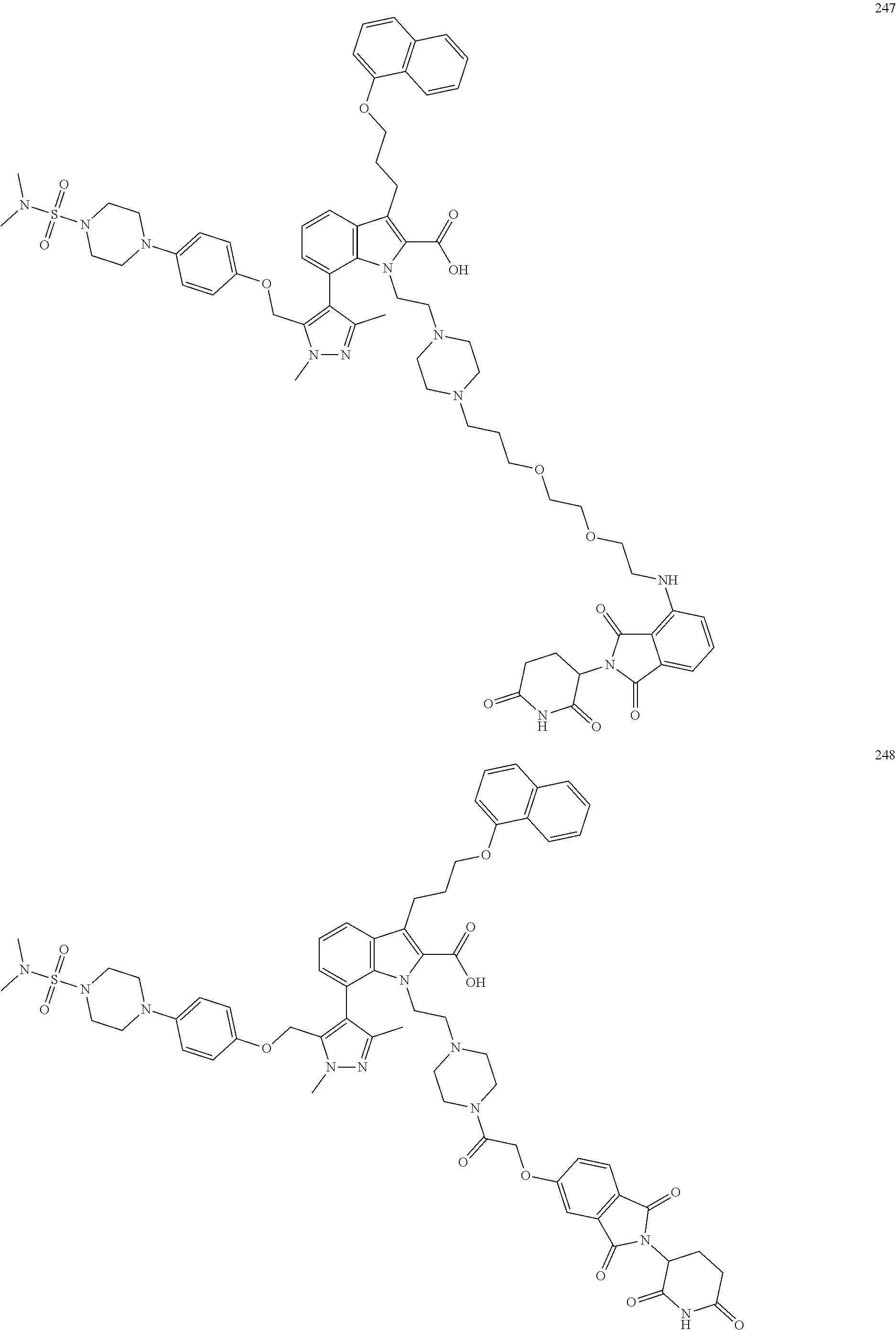 [SMILES: Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(CCCOCCOCCNc4cccc5c4C(=O)N(C4CCC(=O)NC4=O)C5=O)CC3)c12 ; Cc1nn(C)c(COc2ccc(N3CCN(S(=O)(=O)N(C)C)CC3)cc2)c1-c1cccc2c(CCCOc3cccc4ccccc34)c(C(=O)O)n(CCN3CCN(C(=O)COc4ccc5c(c4)C(=O)N(C4CCC(=O)NC4=O)C5=O)CC3)c12]

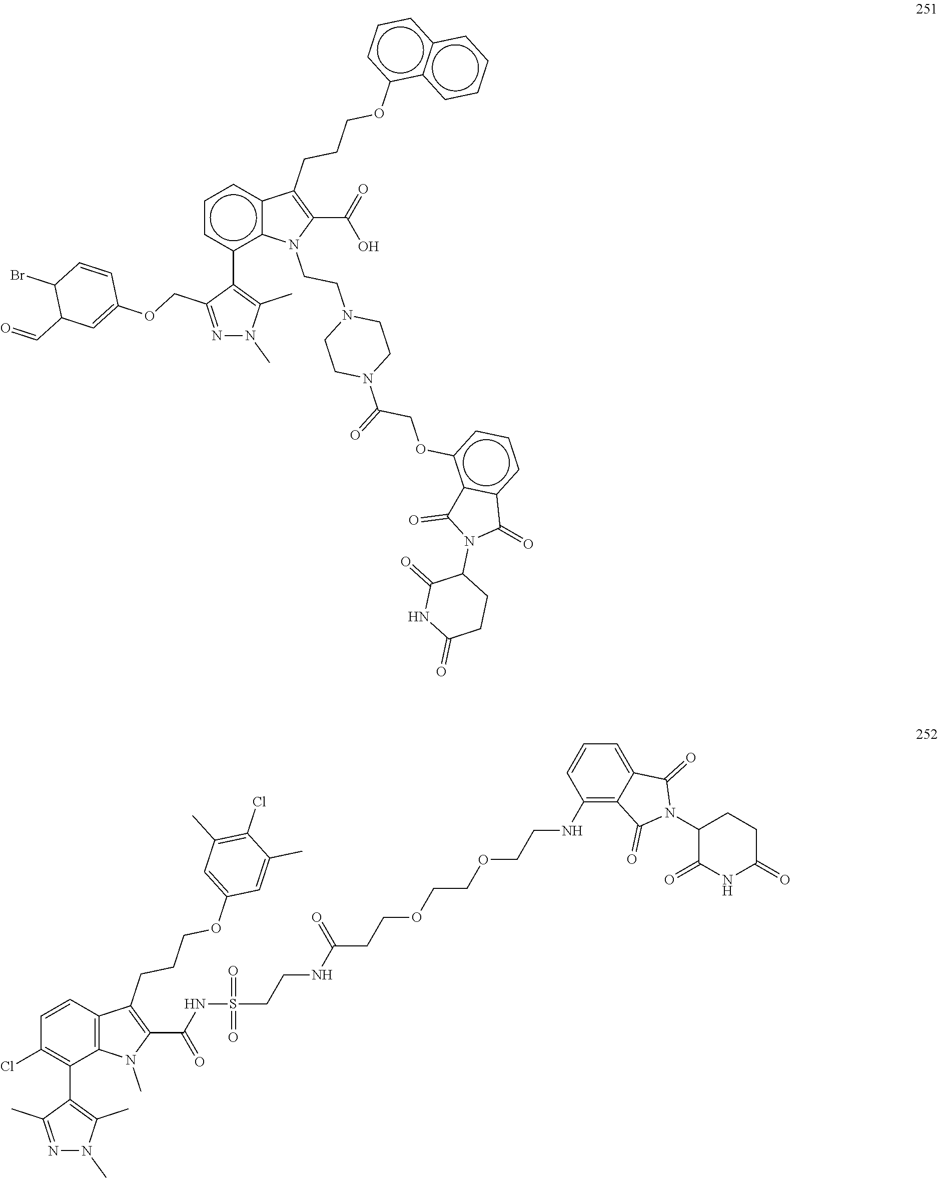

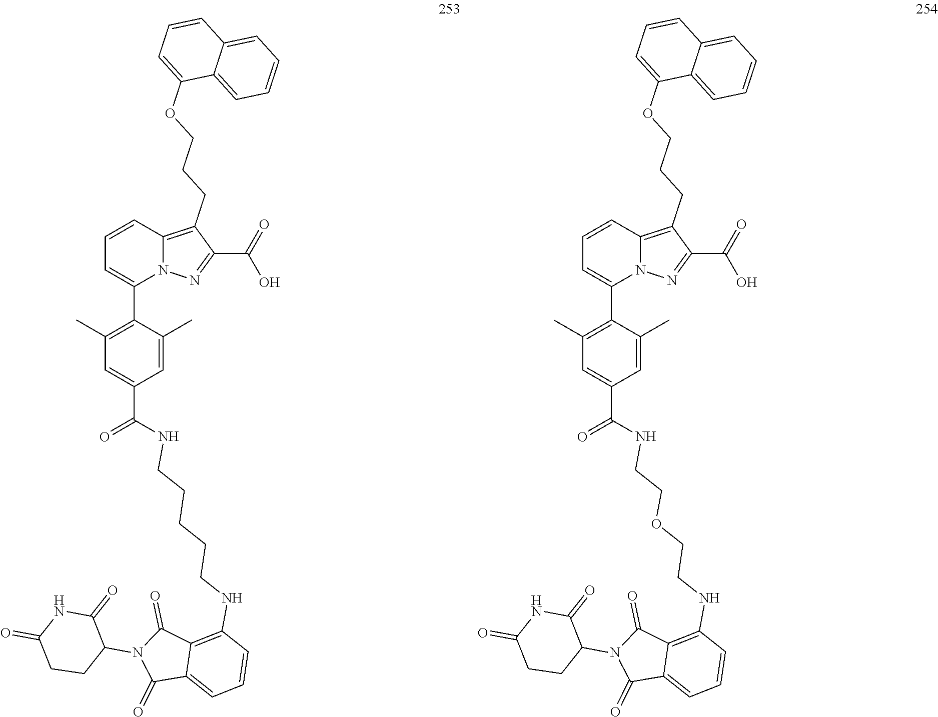 [SMILES: Cc1cc(C(=O)NCCCCCNc2cccc3c2C(=O)N(C2CCC(=O)NC2=O)C3=O)cc(C)c1-c1cccc2c(CCCOc3cccc4ccccc34)c(C(=O)O)nn12 ; Cc1cc(C(=O)NCCOCCNc2cccc3c2C(=O)N(C2CCC(=O)NC2=O)C3=O)cc(C)c1-c1cccc2c(CCCOc3cccc4ccccc34)c(C(=O)O)nn12]

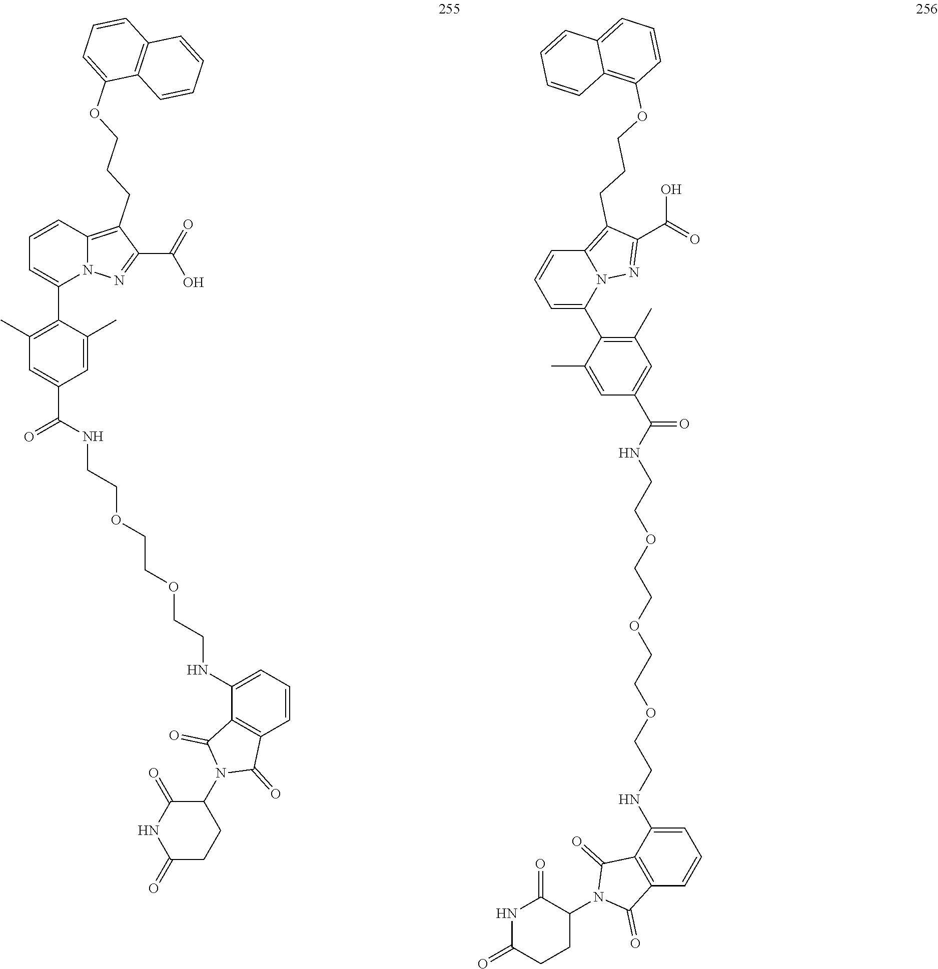 [SMILES: Cc1cc(C(=O)NCCOCCOCCNc2cccc3c2C(=O)N(C2CCC(=O)NC2=O)C3=O)cc(C)c1-c1cccc2c(CCCOc3cccc4ccccc34)c(C(=O)O)nn12 ; Cc1cc(C(=O)NCCOCCOCCOCCNc2cccc3c2C(=O)N(C2CCC(=O)NC2=O)C3=O)cc(C)c1-c1cccc2c(CCCOc3cccc4ccccc34)c(C(=O)O)nn12]

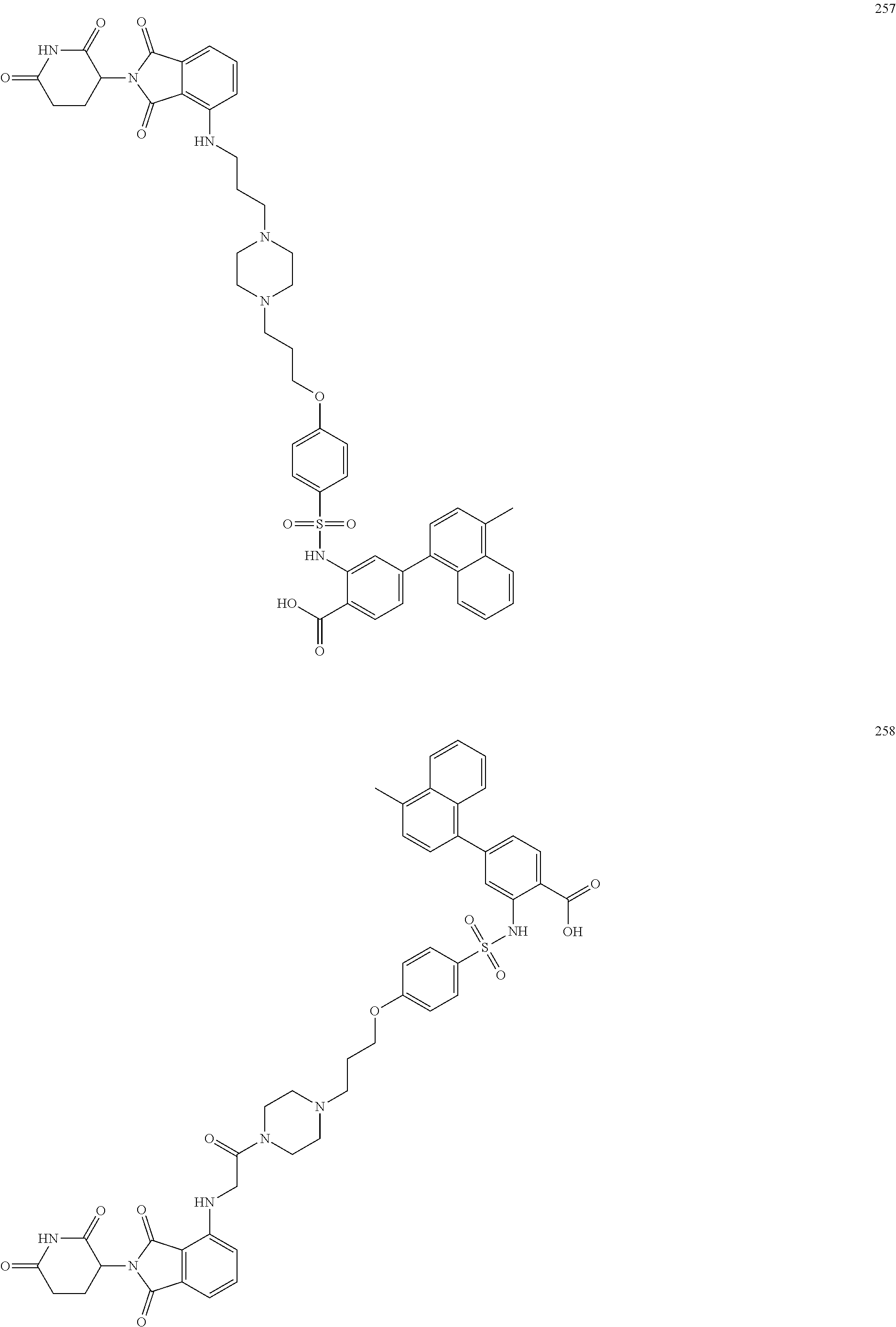 [SMILES: Cc1ccc(-c2ccc(C(=O)O)c(NS(=O)(=O)c3ccc(OCCCN4CCN(CCCNc5cccc6c5C(=O)N(C5CCC(=O)NC5=O)C6=O)CC4)cc3)c2)c2ccccc12 ; Cc1ccc(-c2ccc(C(=O)O)c(NS(=O)(=O)c3ccc(OCCCN4CCN(C(=O)CNc5cccc6c5C(=O)N(C5CCC(=O)NC5=O)C6=O)CC4)cc3)c2)c2ccccc12]

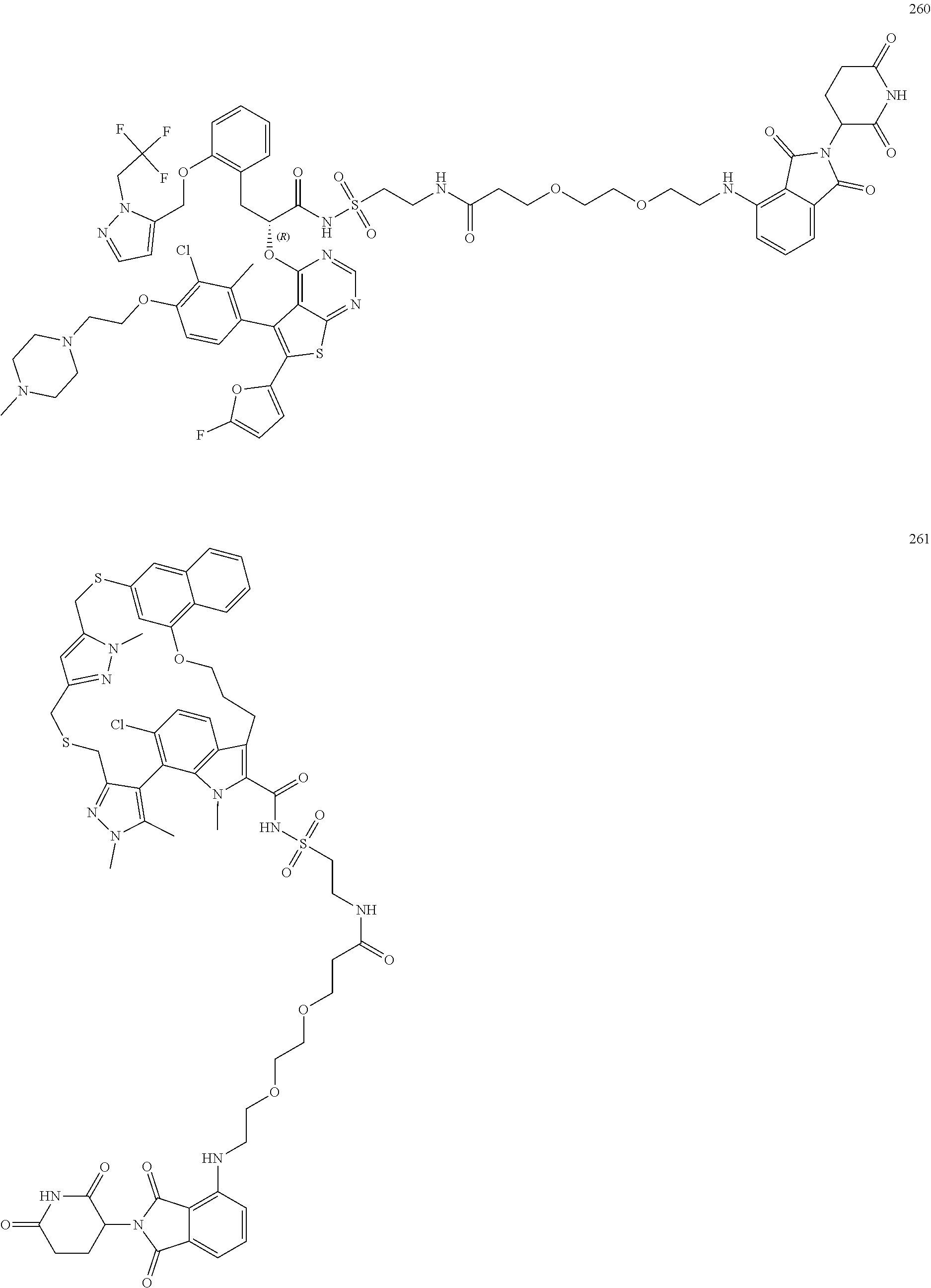

47. The compound of any preceding claim, wherein each alkyl, alkenyl, alkynyl, aryl, heteroaryl and benzyl is unsubstituted.

48. A compound of formula (I)
[MCL-1 ligand moiety]-[linker]-[ligase ligand moiety] (I)
or a salt, solvate, hydrate, isomer or prodrug thereof,
wherein [ligase ligand moiety] is:
(a) Formula (IV) 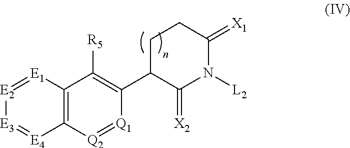
wherein:
each of X₁ and X₂ is independently O or S;
each of Q₁ and Q₂ is independently N or CR⁵, wherein at least one of Q₁ and Q₂ is N;
each of E₁, E₂, E₃ and E₄ is independently N or CR′;
n is 0, 1 or 2;
L₂ is hydrogen, alkyl, alkenyl, aryl, heteroaryl, benzyl, haloalkyl, haloalkenyl, —C(O)R′″, —C(O)OR′″, —C(O)NH₂, —C(O)NHR′″, —C(O)NR′″₂, —OR′″, —NR′″₂, or —S(O)₂R′;
each R⁵ is independently hydrogen, halogen, alkyl, alkenyl, alkynyl, aryl, heteroaryl, benzyl, haloalkyl, haloalkenyl, —NH₂, —NHR′″, —NR″₂, —NR′″C(O)R′″, —NR′″C(O)OR′″, —NO₂, —CN, —C(O)R′″, —C(O)OR′″, —C(O)NH₂, —C(O)NHR′″, —C(O)NR′″₂, —OR′″, —OC(O)R′″, —OC(O)OR′″, —OC(O)NH₂, —OC(O)NHR′″, —OC(O)NR′″₂, —SR′″, —S(O)₂R′″, —S(O)₂OR′″, —S(O)₂NH₂, —S(O)₂NHR′″, —S(O)₂NR′″₂; —O—R²¹, —NH—R²¹, —C(O)—NH—R²¹, or —CH₂—NH—C(O)—R²¹;
each R′ is independently hydrogen, halogen, alkyl, alkenyl, alkynyl, aryl, heteroaryl, benzyl, haloalkyl, haloalkenyl, —NH₂, —NHR′″, —NR′″₂, —NR′″C(O)R′″, —NR′″C(O)OR′″, —NO₂, —CN, —C(O)R′″, —C(O)OR′″, —C(O)NH₂, —C(O)NHR′″, —C(O)NR′″₂, —OR′″, —OC(O)R′″, —OC(O)OR′″, —OC(O)NH₂, —OC(O)NHR′″, —OC(O)NR′″₂, —SR′″, —S(O)₂R′″, —S(O)₂OR′″, S(O)₂NH₂, —S(O)₂NHR′″, —S(O)₂NR′″₂, —R²¹, —O—R²¹, —NH—R²¹, —C(O)—R²¹, —C(O)—NH—R²¹, or —CH₂—NH—C(O)—R²¹;
and
each R′″ is independently hydrogen, alkyl, alkenyl, aryl, heteroaryl, or benzyl;
wherein R²¹ is a bond connected to R¹⁸ of the linker, and wherein Formula (IV) contains a single R²¹; or
(b) Formula (Va) or (Vb): 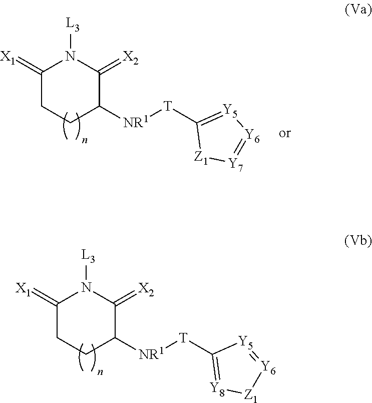
or a pharmaceutically acceptable salt or tautomer thereof,
wherein
each of X₁ and X₂ is independently O or S;
Z₁ is O, S or NR⁶;
T is is C═O or SO₂;
R¹ is hydrogen, alkyl, cycloalkyl, alkenyl, cycloalkenyl, aryl, heteroaryl, or benzyl;
each of Y₅, Y₆, Y₇, and Y₈ is independently N or CR⁷, wherein at least one of Y₅, Y₆ and Y₇ in Formula (Va) is CR⁷, and at least one of Y₅, Y₅ and Y₈ in Formula (Vb) is CR⁷;
n is 0, 1 or 2;
L₃ is hydrogen, alkyl, alkenyl, aryl, heteroaryl, benzyl, haloalkyl, haloalkenyl, —C(O)R″″, —CH₂C(O)OR″″, —C(O)OR″″, —C(O)NH₂, —C(O)NHR″″, —C(O)NR″″₂, —OR″″, —NR″″₂, or —S(O)₂R″″;
each R⁷ is independently hydrogen, halogen, alkyl, cycloalkyl, heterocycloalkyl, alkenyl, cycloalkenyl, alkynyl, aryl, heteroaryl, benzyl, haloalkyl, haloalkenyl, —NH₂, —NHR″″, —NR″″₂, —CH₂NR″″₂, —NR″″C(O)R″″, —NR″″C(O)CH₂NR″″₂, —NR″″C(O)CH₂-heterocycloalkyl, —NR″″C(O)CH(OH)R″″, —CH₂NR″″C(O)OR″″, —NR″″C(O)OR″″, —NR″″SO₂R″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —NHC(S)NHR″″, SR″″, or —S(O)₂R″″, —S(O)₂OR″″, —S(O)₂NH₂, —S(O)₂NHR″″, —S(O)₂NR″″₂, —O—R²¹, —NH—R²¹, —C(O)—NH—R²¹, or —CH₂—NH—C(O)—R²¹;
each R″″ is independently hydrogen, alkyl, cycloalkyl, alkenyl, cycloalkenyl, aryl, heteroaryl, or benzyl;
R⁶ is hydrogen, alkyl, cycloalkyl, alkenyl, cycloalkenyl, alkynyl, aryl, heteroaryl, benzyl, haloalkyl, haloalkenyl, —NH₂, —NHR″″, —NR″″₂, —NR″″C(O)R″″, —N[C(O)R″″]₂, —NR″″C(O)OR″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —SR″″, or —S(O)₂R″″, —S(O)₂OR″″, —S(O)₂NH₂, —S(O)₂NHR″″, —S(O)₂NR″″₂, —R²¹, —C(O)—NH—R²¹, or —CH₂—NH—C(O)—R²¹;
wherein R²¹ is a bond connected to R¹⁸ of the linker, and wherein formula (Va) and formula (Vb) each contain a single R²¹;
wherein when Z₁ is O, then Y₆ is CR⁷ and
wherein when the compound is of Formula (Va), then
(i) when each of Y₅, Y₆ and Y₇ is CR⁷, then at least one of R⁷ is not H;
(ii) when Z₁ is NR⁶, then Y₆ and Y₇ are CR⁷;
(iii) when Z₁ is S, then Y₅ is not C—OMe and Y₆ is not C—OMe;
(iv) when Z₁ is S and Y₅ is C—NHCOMe, then Y₇ is not C—CH₂NR″″C(O)OR″″;
(v) when Z₁ is S and Y₅ is N, then Y₆ is not C—H, C-aryl or C—C(O)OR″″; and
(vi) when Z₁ is S and Y₆ is N, then Y₇ is C—NH₂, C—NHR″″, C—NR″″₂, C—NR″″C(O)OR″″, C—CH₂NR″″C(O)OR″″, C-haloalkyl, C-^{t}Butyl, C—OR″″, C—COOR″″ or C—SR″″; wherein when Y₇ is C—NH₂, C—NHR″″ or C—NR″″₂, then Y₅ is C—H;
and when the compound is of Formula (Vb), then:
(vii) when each of Y₅, Y₆ and Y₈ is CR⁷, then at least one of R⁷ is not H;
(viii) when Z₁ is S, then Y₅ is not C—COOH or C—NHC(O)Me, and Y₈ is not C—Br;
(ix) when Z₁ is S and Y₆ is C—Br, then Y₈ is C—OR″″
(x) when Z₁ is S, Y₅ is N and Y₆ is C—H or C—NH₂, then Y₈ is not C—H
(xi) when Z₁ is S and Y₅ is N, then Y₆ is not C— halogen, C-alkyl, C-cycloalkyl, C-aryl, C-heteroaryl, C—CH₂NH₂, C—COOalkyl, or C—NHC(O)alkyl; (xii) when Z₁ is NR⁶, then Y₅, Y₆ and Y₈ are CR⁷, or
(c) Formula (IIa) or (IIb): 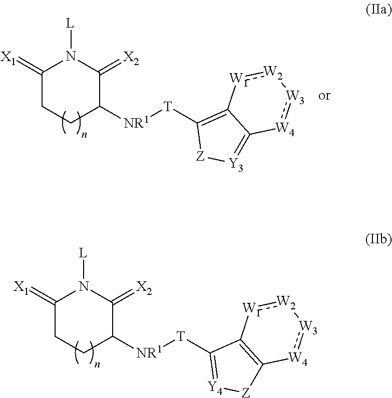
wherein
each of X₁ and X₂ is independently O or S;
Z is O, S or NR²;
T is C═O or SO₂;
Y₃ is N or CR;
Y₄ is N or CR; [symbol]
indicates a single or double bond, wherein
when each [symbol] is a double bond, each of W₁, W₂, W₃ and W₄ is independently N or CR^{a}, wherein at least one of W₁, W₂, W₃ and W₄ is N, and
when each [symbol] is a single bond, W₁, W₂, W₃ and W₄ are each CR^{a}₂ and Y₄ is CR;
n is 0, 1 or 2;
L is hydrogen, alkyl, alkenyl, aryl, heteroaryl, benzyl, haloalkyl, haloalkenyl, —C(O)R^{h}, —C(O)OR^{h}, —C(O)NH₂, —C(O)NHR^{h}, —C(O)NR^{h}₂, —OR^{h}, —NR^{h}₂, or —S(O)₂R^{h};
each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH₂R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, or —S(O)₂R^{h}, —S(O)₂OR^{h}, —S(O)₂NH₂, —S(O)₂NHR^{h}, or —S(O)₂NR^{h}₂;
each R^{a} is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, —S(O)₂NR^{h}₂, —O—R²¹, —NH—R²¹, —C(O)—NH—R²¹, or —CH₂—NH—C(O)—R²¹;
each R^{h} is independently hydrogen, alkyl, cycloalkyl, heterocycloalkyl, alkenyl, cycloalkenyl, aryl, heteroaryl, or benzyl;
R² is hydrogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —N[C(O)R^{h}]₂, —NR^{h}C(O)OR^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, or —S(O)₂NR^{h}₂; and
R¹ is hydrogen, alkyl, cycloalkyl, alkenyl, cycloalkenyl, aryl, heteroaryl, or benzyl;
R²¹ is a bond connected to R¹⁸ of the linker, and wherein formula (IIa) and formula (IIb) each contain a single R²¹;
wherein when each [symbol] is a double bond, Z is NR², R² is hydrogen, and each R^{a} is hydrogen, then W₄ is CR^{a};
wherein
[MCL-1 ligand moiety] is a compound of Formula (A), Formula (B) or Formula (C) 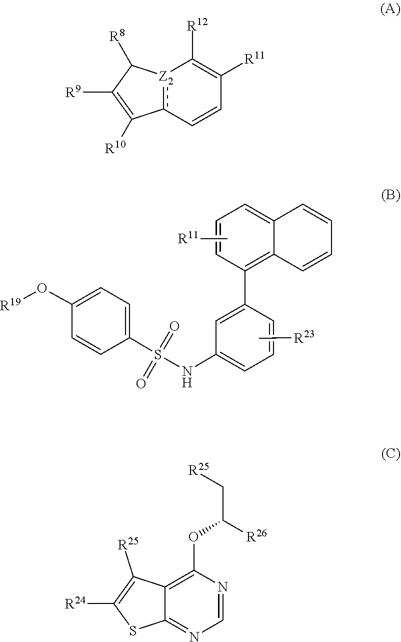
wherein [symbol]
is a single bond or a double bond;
R⁸ is H, R¹⁹, or C₁-C₆ alkyl optionally substituted with morpholine;
R⁹ is —C(O)OH, —C(O)OC₁-C₆alkyl, —C(O)NH₂, —C(O)OR¹⁹ or —C(O)NHR¹⁹,
R¹⁰ is —C₂₋₅alkyl-O—R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹¹ is H, halogen or C₁-C₆ alkyl,
R¹² is H, 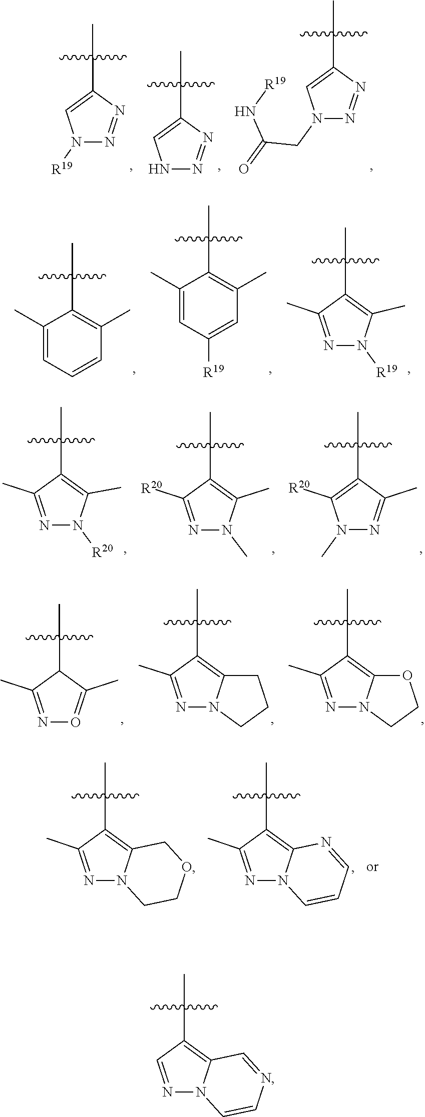
wherein R²⁰ is Me, —CH₂—OMe, —CH₂—O-bromobenzaldehyde, or 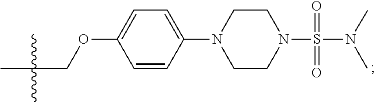 [SMILES: *COc1ccc(N2CCN(S(=O)(=O)N(C)C)CC2)cc1]
or when R¹² is 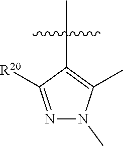
and R¹⁰ is —O-naphthyl substituted with —O— or —S—, then R²⁰ is 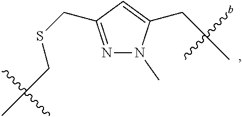 [SMILES: *CSCc1cc(C*)n(C)n1]
wherein [symbol] indicates attachment to —O— or —S— of R¹⁰;
and wherein
R¹⁹ is a bond connected to R¹⁴ of the linker;
R²³ is —C(O)OH or —C(O)OC₁-C₆alkyl;
Z₂ is N or C, wherein when Z₂ is N, then [symbol] is a single bond; and when Z₂ is C, then [symbol] is a double bond,
R²⁴ is furan optionally substituted with at least one halogen,
each R²⁵ is independently phenyl substituted with —OR²⁸ and optionally further substituted with at least one substituent selected from halogen and C₁-C₆ alkyl;
R²⁶ is —C(O)OR¹⁹ or —C(O)NHR¹⁹; and
each R²⁸ is independently —C₁₋₃alkyl-(N-alkyl piperazine) or —C₁₋₃alkyl-(N-haloalkylpyrazole)
and wherein each of Formula (A), Formula (B) and Formula (C) contains a single R¹⁹;
and wherein [linker] has the following formula
R¹⁴—R¹⁵—R¹⁶—R¹⁷—R¹⁸
wherein
R¹⁴ is —C₁₋₆alkyl, —C₂₋₆alkenyl, —C₂₋₆alkynyl, —C₁₋₆ alkyl-N(C₁₋₆ alkyl)-, —C(O)—, —SO₂— or is absent
R¹⁵ is cycloalkyl, heterocycloalkyl, aryl, heteroaryl, —C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, (C₂H₄—O)ₓ, (C₃H₆—O)ₓ, or is absent
x is 1-10
y is 2-10
R¹⁸ is —C₁₋₆ alkyl, heterocycloalkyl, or is absent
wherein at least one of R¹⁴-R¹⁸ is present.

49. The compound of claim 448, wherein each alkyl, alkenyl, alkynyl, aryl, heteroaryl and benzyl groups is unsubstituted.

50. The compound of any one of claims 48-49, wherein each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR″″, —NR″″₂, —NR″″C(O)R″″, —NR″″C(O)CH(OH)R″″, —NR″″C(O)OR″″, —NR″″SO₂R″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —SR″″, or —S(O)₂R″″, —S(O)₂OR″″, —S(O)₂NH₂, —S(O)₂NHR″″, or —S(O)₂NR″″₂, —O—R²¹, —NH—R²¹, —C(O)—NH—R²¹, or —CH₂—NH—C(O)—R²¹.

51. The compound of any one of claims 48-50, wherein each R′ is independently hydrogen, halogen, alkyl, alkenyl, alkynyl, aryl, heteroaryl, benzyl, haloalkyl, haloalkenyl, —NH₂, —NHR′″, —NR′″₂, —NR′″C(O)R′″, —NR′″C(O)OR′″, —NO₂, —CN, —C(O)R′″, —C(O)OR′″, —C(O)NH₂, —C(O)NHR′″, —C(O)NR′″₂, —OR′″, —OC(O)R′″, —OC(O)OR′″, —OC(O)NH₂, —OC(O)NHR′″, —OC(O)NR′″₂, —SR′″, —S(O)₂R′″, —S(O)₂OR′″, S(O)₂NH₂, —S(O)₂NHR′″, —S(O)₂NR′″₂, —O—R²¹, —NH—R²¹, —C(O)—NH—R²¹, or —CH₂—NH—C(O)—R²¹.

52. The compound of any one of claims 48-51, wherein R¹ is hydrogen.

53. The compound of any one of claims 48-52, wherein R⁶ is hydrogen.

54. The compound of any one of claims 48-53, wherein when Z₁ is S in Formula (Vb), then Y₅ is not C—NHC(O)R″″ or —C(O)OR″″.

55. The compound of any one of claims 48-54, wherein Z₁ is NR⁶.

56. The compound of any one of claims 48-55, wherein [ligase ligand moiety] is of Formula (Va) and Y₅, Y₆ and Y₇ are each CR⁷.

57. The compound of claim 56, wherein
Y₅ is —C—NHC(O)R″″,
Y₆ is CH, and
Y₇ is CH or CCl.

58. The compound of claim 57, wherein:
L₃ is hydrogen;
Z₁ is S;
R′ is hydrogen;
T is C═O; and
Y₇ is CH.

59. The compound of any one of claims 48-55, wherein the compound is of Formula (Vb) and Y₅, Y₆ and Y₈ are each CR⁷.

60. The compound of claim 59, wherein:
L₃ is hydrogen;
Z₁ is S;
R¹ is H;
T is C═O;
Y₅ is CH, C—OR″″, CCl, C—CN, or C—NHC(O)R″″;
Y₆ is CH, CCl, C-alkyl, C-cycloalkyl, or C-haloalkyl; and
Y₈ is CH, C—OR″″, C—NHC(O)R″″, C—NHC(O)OR″″, C—NHR″″, C—NH₂, or C—NHSO₂R″″;
wherein, when Y₅ is CCl, then Y₆ is CH, C-alkyl, C-cycloalkyl, or C-haloalkyl;
optionally wherein each R″″ is independently alkyl, cycloalkyl, aryl or benzyl.

61. The compound of claim 60, wherein:
Y₅ is CH;
Y₆ is CH or CCl; and
Y₈ is C—OR″″ or C—NH₂, optionally C—OMe or C—NH₂.

62. The compound of any one of claims 48-61, wherein Z is NR².

63. The compound of any one of claims 48-61, wherein Z is S.

64. The compound of any one of claims 48-62, wherein each [symbol] s a double bond.

65. The compound of any one of claims 48-64, wherein L is hydrogen.

66. The compound of claim 64 or 65, wherein one of W₁, W₂, W₃ and W₄ is N, and the remaining three of W₁, W₂, W₃ and W₄ are each CR^{a}; optionally wherein W₄ is CR^{a}.

67. The compound of claim 64 or 65, wherein two of W₁, W₂, W₃ and W₄ is N, and the remaining two of W₁, W₂, W₃ and W₄ are each CR^{a}.

68. The compound of claim 64 or 65, wherein one of W₁, W₂, W₃ and W₄ is CR^{a}, and the remaining three of W₁, W₂, W₃ and W₄ are each N.

69. The compound of any one of claims 48-68, wherein each R is independently hydrogen, halogen or —NR^{h}C(O)R^{h}.

70. The compound of any one of claims 48-69, wherein [ligase ligand moiety] is:

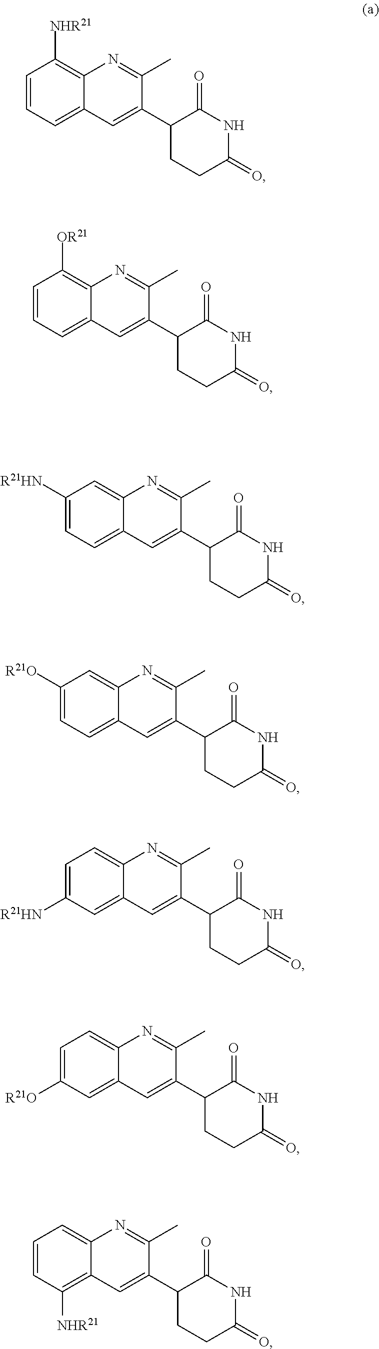

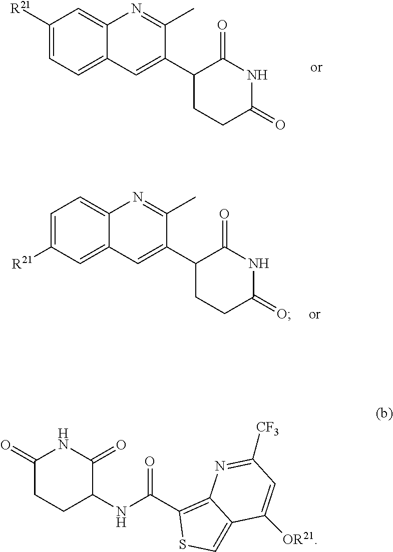

71. The compound of any one of claims 48-70, wherein E₁, E₂, E₃ and E₄ are each CR′.

72. The compound of any one of claims 48-71, wherein one of E₁, E₂, E₃ and E₄ is N and the remaining three of E₁, E₂, E₃ and E₄ are each CR′.

73. The compound of any one of claims 48-72, wherein Q₁ is CR⁵

74. The compound of any one of claims 48-72, wherein Q₂ is CR⁵

75. The compound of any one of claims 48-74, wherein R¹⁴ is —C₁₋₆alkyl, —C₂₋₆alkenyl, —C₂₋₆alkynyl, —C(O)—, —SO₂— or is absent.

76. The compound of any one of claims 48-75, wherein R¹⁵ is cycloalkyl, heterocycloalkyl, aryl, heteroaryl, —C₁₋₆ alkyl-NH—, -cycloalkyl-NH— or is absent.

77. The compound of any one of claims 48-76, wherein
R¹⁴ is —C₁₋₆ alkyl, —SO₂— or is absent
R¹⁵ is piperazine, bridged piperazine, piperazine N-oxide, piperazine cation, —C₁₋₆ alkyl-NH—, 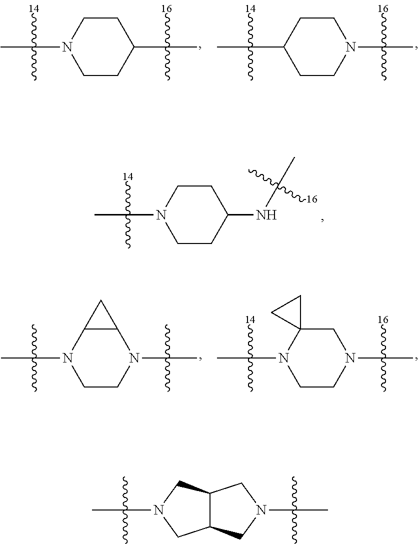
or is absent, wherein [symbol] indicates attachment to R¹⁴ and [symbol] indicates attachment to R¹⁶,
R¹⁶ is —C₁₋₆ alkyl, —C(O)—, —C(O)—NH—, —CH₂—C(O)—NH— or is absent
R¹⁷ is —CH₂(C₂H₄—O)_{y}, (C₂H₄—O)ₓ, (C₃H₆—O)ₓ, or is absent
x is 1-6
y is 2-6
R¹⁸ is —C₁₋₆ alkyl, piperazine, or is absent
wherein at least one of R¹⁴-R¹⁸ is present

78. The compound of any one of claims 48-77, wherein R¹⁸ is —C₁₋₆ alkyl or is absent.

79. The compound of any one of claims 48-78, wherein when R¹⁵ is piperazine, bridged piperazine, piperazine N-oxide, piperazine cation, —C₁₋₆ alkyl-NH—, 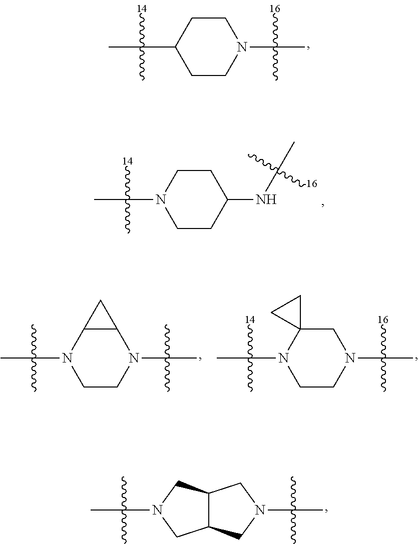
then R¹⁴ is —C₁₋₆ alkyl.

80. The compound of any one of claims 48-79, wherein
R¹⁴ is —C₁₋₆ alkyl,
R¹⁵ is piperazine, bridged piperazine, piperazine N-oxide, 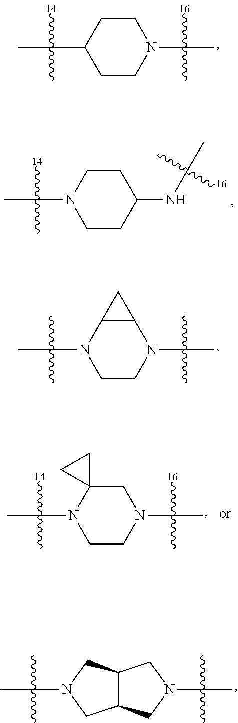
R¹⁶ is —C(O)—, —CH₂—C(O)—NH—, or is absent
R¹⁷ is —CH₂(C₂H₄—O)_{y}, (C₂H₄—O)ₓ, (C₃H₆—O)ₓ, or is absent
R¹⁸ is —C₁₋₆ alkyl,
wherein when R¹⁶ and R¹⁷ are absent, R¹⁸ is —C₃₋₆ alkyl.

81. The compound of claim 80, wherein
R¹⁴ is —C₂ alkyl,
x is 1, 2 or 6
y is 2.

82. The compound of claim 80, wherein
R¹⁵ is piperazine,
R¹⁶ is —C(O)—,
R¹⁷ is absent.

83. The compound of claim 82, wherein
R¹⁴ is —C₂ alkyl,
R¹⁸ is —C₁₋₂ alkyl.

84. The compound of any one of claims 48-78, wherein when R¹⁴ is —SO₂—, at least two of R¹⁵-R¹⁸ are present, and at least one of R¹⁵—R¹⁸ is not C₁₋₆ alkyl.

85. The compound of any one of claims 48-78, wherein
R¹⁴ is —SO₂—
R¹⁵ is —C₁₋₆ alkyl-NH—
R¹⁶ is —C(O)—
R¹⁷ is —CH₂(C₂H₄—O)_{y}, (C₂H₄—O)ₓ or is absent
R¹⁸ is —C₂₋₄ alkyl.

86. The compound of claim 85, wherein
R¹⁵ is —C₂alkyl-NH—
x is 1 or 2
y is 1
R¹⁸ is —C₂₋₄ alkyl

87. The compound of any one of claims 48-78 wherein
R¹⁴ is absent
R¹⁵ is absent
R¹⁶ is —C(O)—NH—, or is absent
R¹⁷ is —CH₂(C₂H₄—O)_{y}, (C₂H₄—O)ₓ, (C₃H₆—O)ₓ, or is absent
R¹⁸ is —C₁₋₆ alkyl.

88. The compound of any one of claims 48-87, wherein at least one of R¹⁴-R¹⁸ is not —C₁₋₆ alkyl.

89. The compound of claim 87 or claim 88, wherein
x is 1, 2 or 3
y is 2
R¹⁸ is —C₂₋₆ alkyl.

90. The compound of any one of claims 48-89, wherein when R¹⁵ is —C₁₋₆ alkyl-NH—, at least one of R¹⁶—R¹⁸ is present.

91. The compound of any one of claims 48-90 wherein when R¹⁷ is —CH₂(C₂H₄—O)_{y}, (C₂H₄—O)ₓ or (C₃H₆—O)ₓ, at least one of R¹⁴—R¹⁶ and R¹⁸ is present, wherein at least one of R¹⁴ and R¹⁸ is not —C₁₋₆ alkyl.

92. The compound of any one of claims 48-91, wherein [linker] is selected from 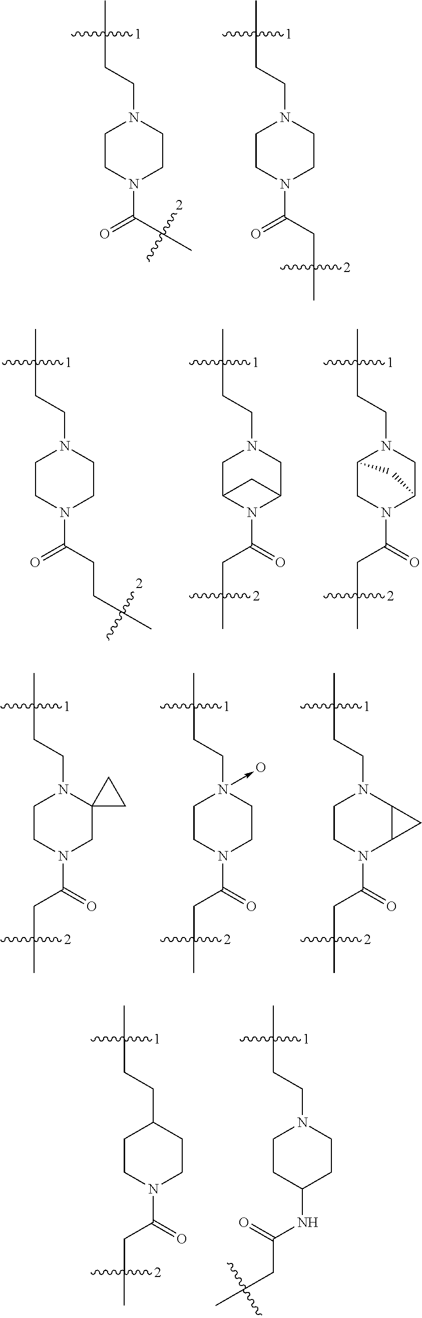 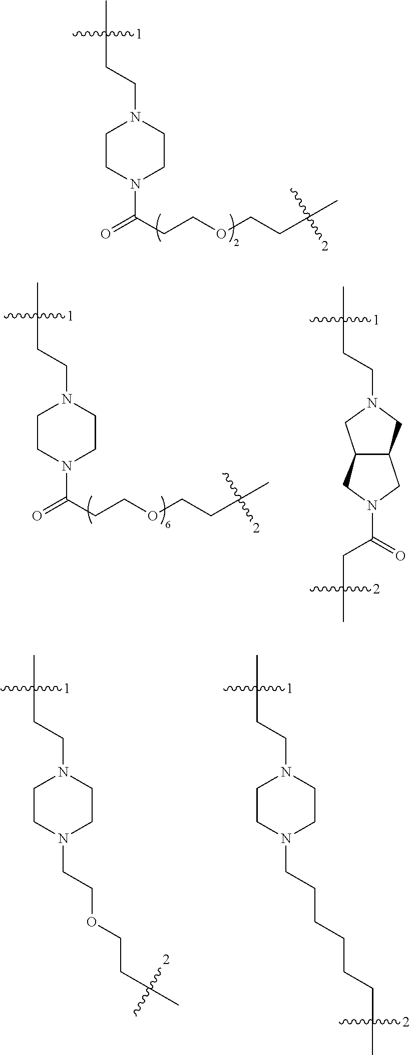 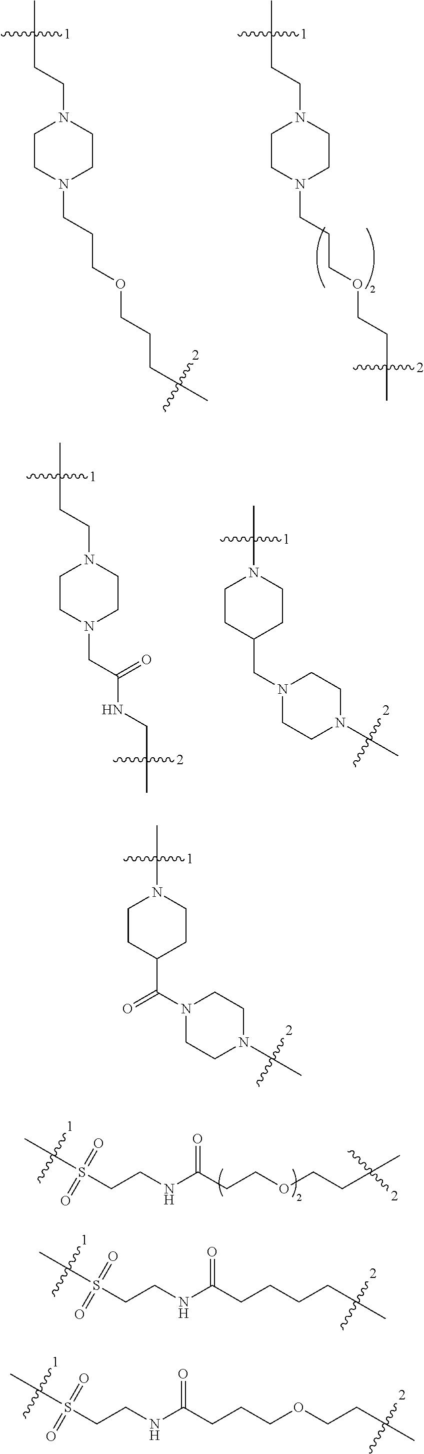 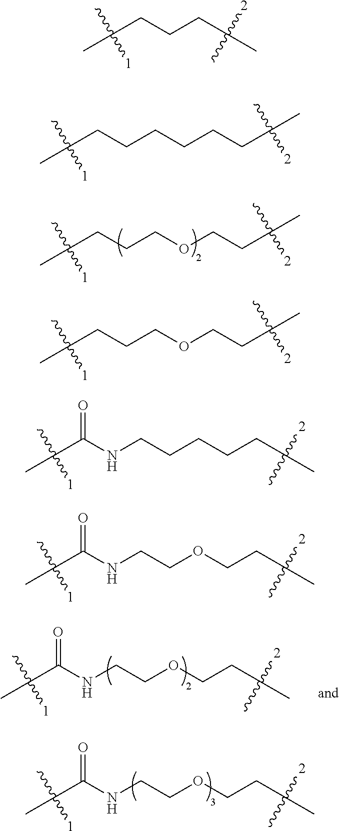
wherein [symbol]
indicates attachment to [MCL-1 ligand moiety] and [symbol]
indicates attachment to [ligase ligand moiety].

93. The compound of any one of claims 48-92, wherein [linker] is 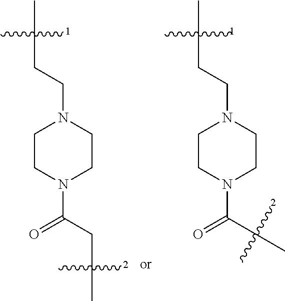
wherein [symbol]
indicates attachment to [MCL-1 ligand moiety] and [symbol]
indicates attachment to [ligase ligand moiety].

94. The compound of any one of claims 48-93, wherein R¹⁰ is —C₂₋₅alkyl-O—R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the naphthyl is optionally substituted with —O— or —S—.

95. The compound of any one of claims 48-94, wherein R¹² is H,

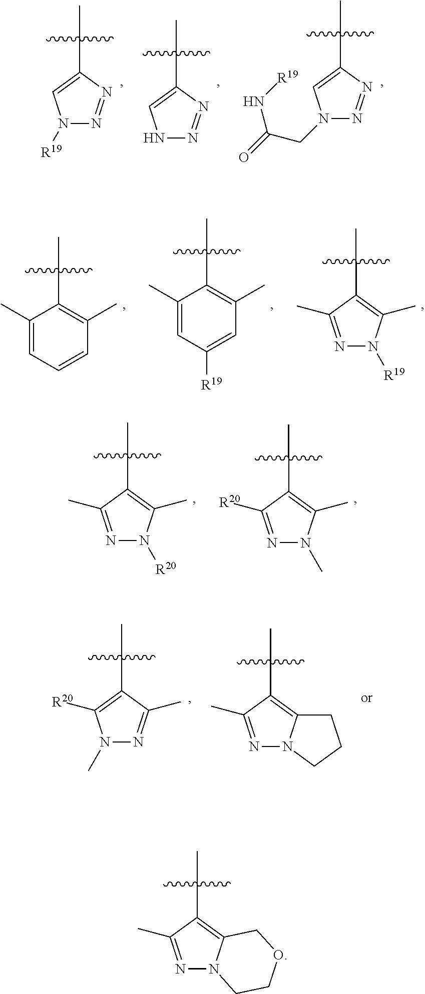

96. The compound of any one of claims 48-95, wherein R²⁰ is Me, —CH₂—O-bromobenzaldehyde, or

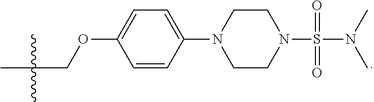 [SMILES: *COc1ccc(N2CCN(S(=O)(=O)N(C)C)CC2)cc1]

97. The compound of any one of claims 48-96 wherein when R⁸ is H, R¹³ is

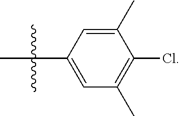 [SMILES: *c1cc(C)c(Cl)c(C)c1]

98. The compound of any one of claims 48-978, wherein
R⁸ is H, R¹⁹, methyl, or —CH₂CH₂-morpholine;
R⁹ is —C(O)OH or —C(O)NHR¹⁹,
R¹⁰ is —C₃H₆O—R¹³,
wherein R¹³ is 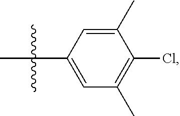 [SMILES: *c1cc(C)c(Cl)c(C)c1]
tetraline, or naphthyl optionally substituted with fluorine;
R¹¹ is H, Cl, F or methyl,
R¹² is 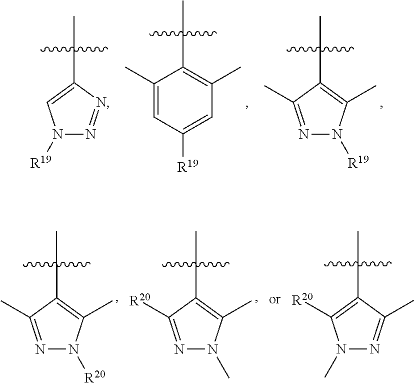
wherein R²⁰ is Me, —CH₂—O-bromobenzaldehyde, or

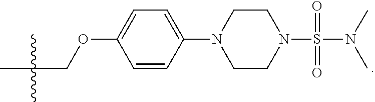 [SMILES: *COc1ccc(N2CCN(S(=O)(=O)N(C)C)CC2)cc1]

99. The compound of claim 98, wherein
R⁸ is R¹⁹ or methyl;
R¹⁰ is —C₃H₆O—R³, wherein R¹³ is naphthyl optionally substituted with fluorine;
R¹¹ is Cl or F,
R¹² is

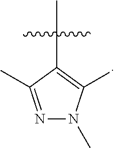

100. The compound of any one of claims 48-99 wherein Z₂ is C and [symbol] is a double bond.

101. The compound of any one of claims 48-100, wherein [MCL-1 ligand moiety] is

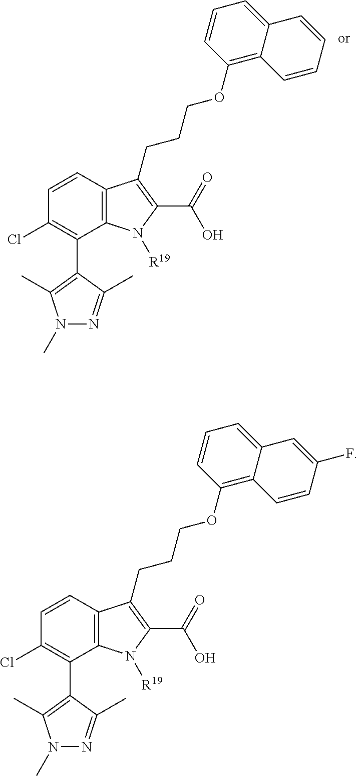

102. The compound of claim 48, wherein the compound is selected from:

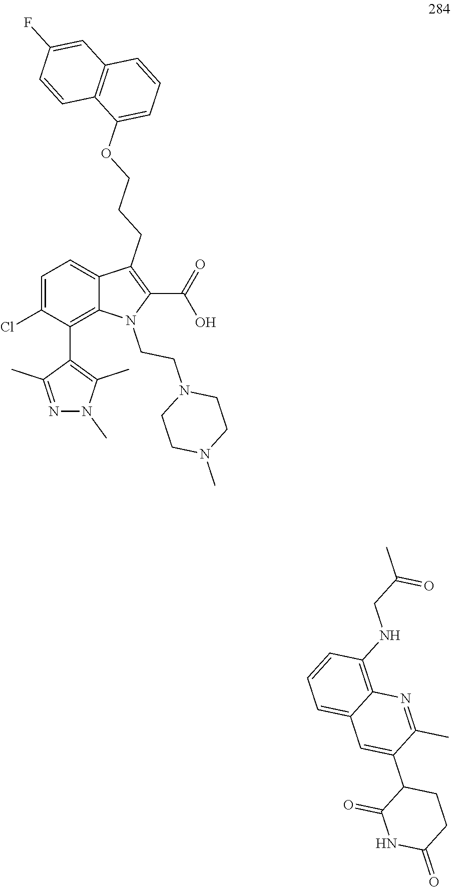 [SMILES: Cc1nn(C)c(C)c1-c1c(Cl)ccc2c(CCCOc3cccc4cc(F)ccc34)c(C(=O)O)n(CCN3CCN(C)CC3)c12 ; CC(=O)CNc1cccc2cc(C3CCC(=O)NC3=O)c(C)nc12]

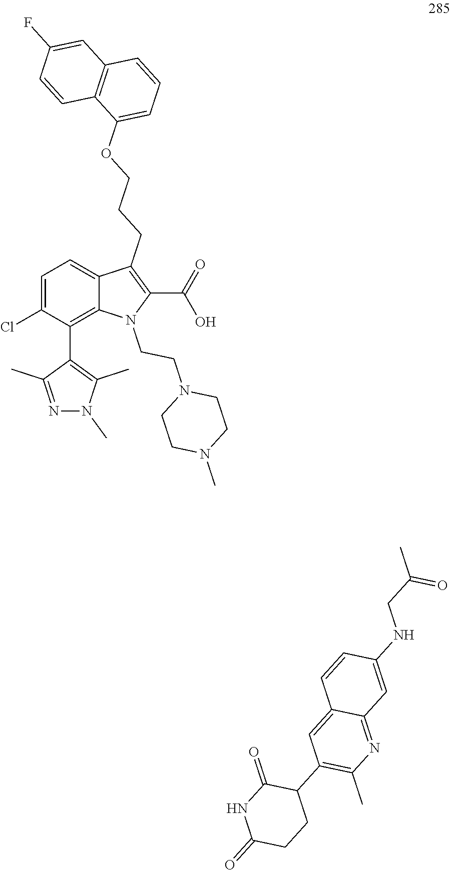 [SMILES: Cc1nn(C)c(C)c1-c1c(Cl)ccc2c(CCCOc3cccc4cc(F)ccc34)c(C(=O)O)n(CCN3CCN(C)CC3)c12 ; CC(=O)CNc1ccc2cc(C3CCC(=O)NC3=O)c(C)nc2c1]

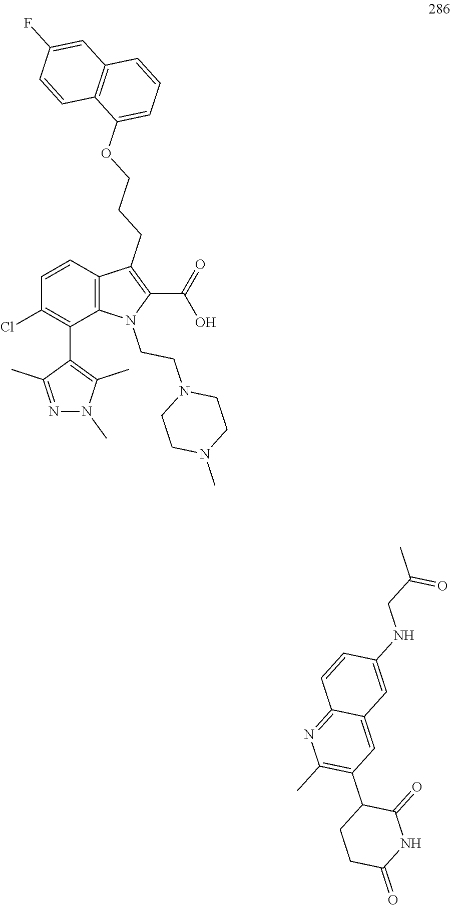 [SMILES: Cc1nn(C)c(C)c1-c1c(Cl)ccc2c(CCCOc3cccc4cc(F)ccc34)c(C(=O)O)n(CCN3CCN(C)CC3)c12 ; CC(=O)CNc1ccc2nc(C)c(C3CCC(=O)NC3=O)cc2c1]

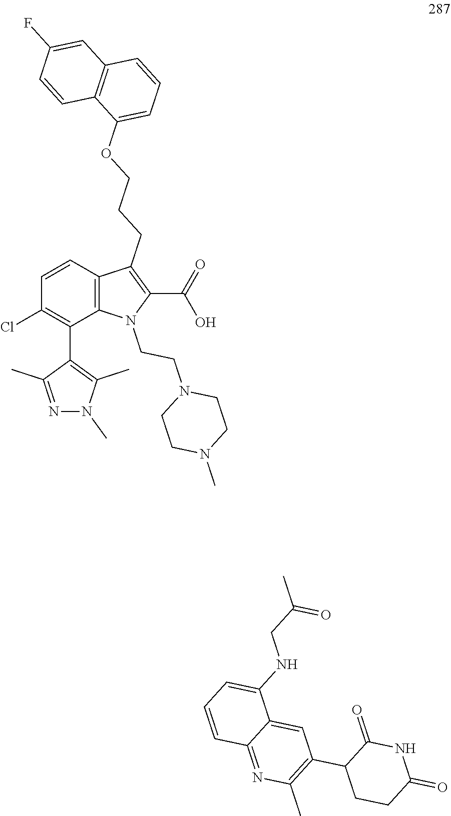 [SMILES: Cc1nn(C)c(C)c1-c1c(Cl)ccc2c(CCCOc3cccc4cc(F)ccc34)c(C(=O)O)n(CCN3CCN(C)CC3)c12 ; CC(=O)CNc1cccc2nc(C)c(C3CCC(=O)NC3=O)cc12]

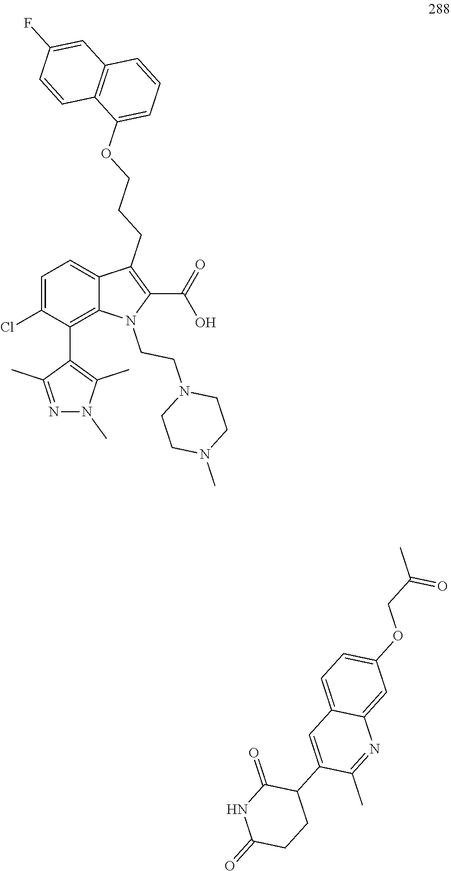 [SMILES: Cc1nn(C)c(C)c1-c1c(Cl)ccc2c(CCCOc3cccc4cc(F)ccc34)c(C(=O)O)n(CCN3CCN(C)CC3)c12 ; CC(=O)COc1ccc2cc(C3CCC(=O)NC3=O)c(C)nc2c1]

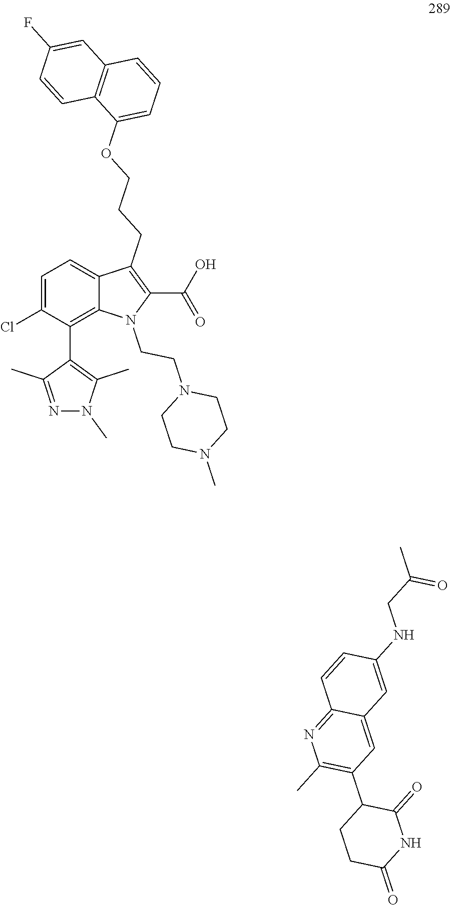 [SMILES: Cc1nn(C)c(C)c1-c1c(Cl)ccc2c(CCCOc3cccc4cc(F)ccc34)c(C(=O)O)n(CCN3CCN(C)CC3)c12 ; CC(=O)CNc1ccc2nc(C)c(C3CCC(=O)NC3=O)cc2c1]

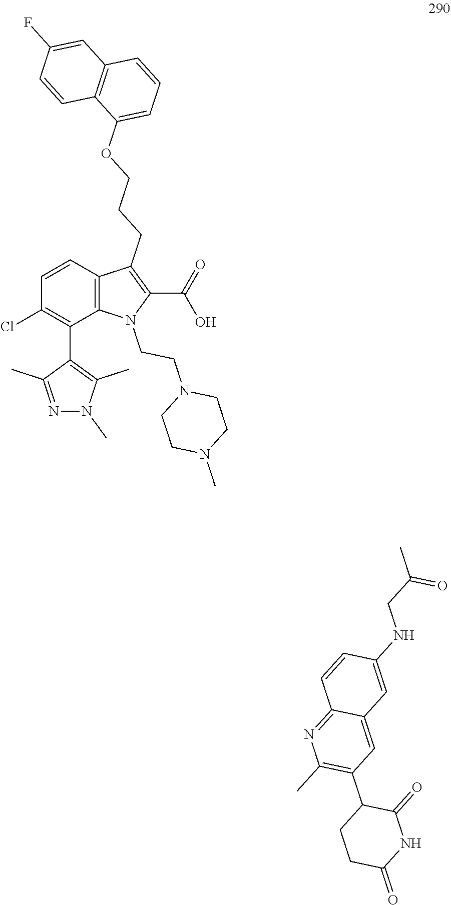 [SMILES: Cc1nn(C)c(C)c1-c1c(Cl)ccc2c(CCCOc3cccc4cc(F)ccc34)c(C(=O)O)n(CCN3CCN(C)CC3)c12 ; CC(=O)CNc1ccc2nc(C)c(C3CCC(=O)NC3=O)cc2c1]

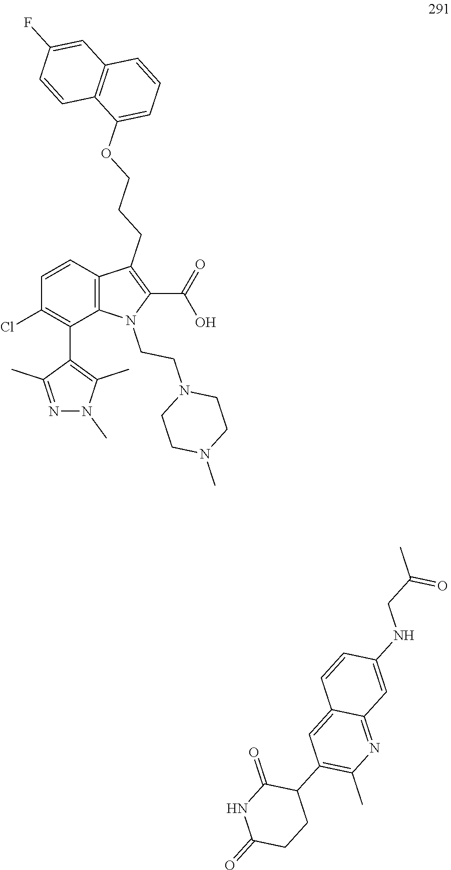 [SMILES: Cc1nn(C)c(C)c1-c1c(Cl)ccc2c(CCCOc3cccc4cc(F)ccc34)c(C(=O)O)n(CCN3CCN(C)CC3)c12 ; CC(=O)CNc1ccc2cc(C3CCC(=O)NC3=O)c(C)nc2c1]

103. The compound of claim 48, wherein the compound is:

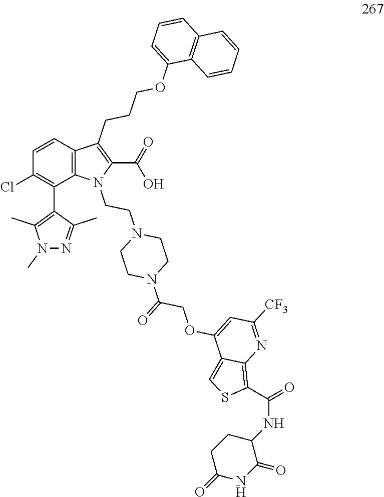 [SMILES: Cc1nn(C)c(C)c1-c1c(Cl)ccc2c(CCCOc3cccc4ccccc34)c(C(=O)O)n(CCN3CCN(C(=O)COc4cc(C(F)(F)F)nc5c(C(=O)NC6CCC(=O)NC6=O)scc45)CC3)c12]

104. A compound of formula (I)
[MCL-1 ligand moiety]-[linker]-[ligase ligand moiety] (I)
or a salt, solvate, hydrate, isomer or prodrug thereof,
wherein [ligase ligand moiety] is:
(a) Formula (11): 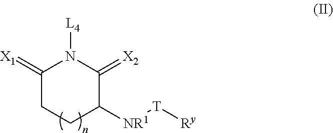
wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L₄ is hydrogen, alkyl, alkenyl, aryl, heteroaryl, benzyl, haloalkyl, haloalkenyl, —C(O)H, —C(O)R″, —C(O)OH, —C(O)OR″, —C(O)NH₂, —C(O)NHR″, —C(O)NR″₂, —OH, —OR″, —NH₂, —NHR″, —NR″₂, —S(O)₂H or —S(O)₂R″;
R^{y} is selected from 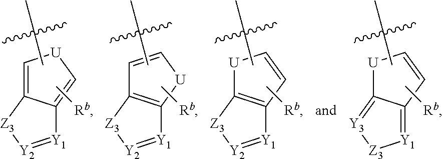
wherein [symbol] indicates attachment to T,
Z₃ is O, S or NR³;
U is O, S, NR^{b} or CR^{b}₂;
each of Y₁, Y₂ and Y₃ is independently N or CR^{d};
each R^{d} is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OH, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, —S(O)₂NR″₂, —O—R²¹, —NH—R²¹, —C(O)—NH—R²¹, or —CH₂—NH—C(O)—R²¹;
each R^{b} is independently hydrogen, halogen, alkyl, cycloalkyl, alkenyl, cycloalkenyl, alkynyl, aryl, heteroaryl, benzyl, haloalkyl, haloalkenyl, —NH₂, —NHR″, —NR″₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OH, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, or —S(O)₂NR″₂;
each R³ is independently hydrogen, alkyl, cycloalkyl, alkenyl, cycloalkenyl, alkynyl, aryl, heteroaryl, benzyl, haloalkyl, haloalkenyl, —NH₂, —NHR″, —NR″₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OH, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, —S(O)₂NR″₂, —R²¹, —C(O)—NH—R²¹, or —CH₂—NH—C(O)—R²¹;
each R″ is independently alkyl, cycloalkyl, alkenyl, cycloalkenyl, aryl, heteroaryl, or benzyl;
R²¹ is a bond connected to R¹⁸ of the linker, wherein Formula (II) contains a single R²¹;
wherein,
(i) when R^{v} is 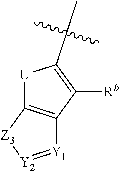
then Y₂ is CR^{d}; and
(ii) when R^{v} is 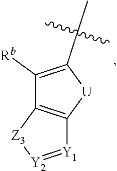
then R^{b} in CR^{b}₂ is not hydrogen
or
(b) Formula (III): 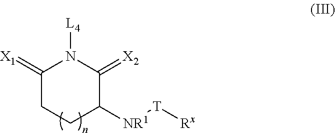
wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L₁ is hydrogen, alkenyl, aryl, heteroaryl, benzyl, haloalkyl, haloalkenyl, —C(O)H, —C(O)R″, —C(O)OH, —C(O)OR″, —CH₂C(O)OR″, —C(O)NH₂, —C(O)NHR″, —C(O)NR″₂, —OH, —OR″, —NH₂, —NHR″, —NR″₂, —S(O)₂H or —S(O)₂R″;
R^{x} is selected from 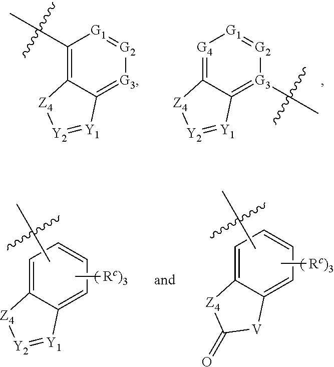
wherein [symbol] indicates attachment to T,
Z₄ is O, S or NR⁴;
V is CR^{f}₂, NR⁴ or S;
each of G₁, G₂, G₃ and G₄ is independently N or CR^{c},
each of Y₁ and Y₂ is independently N or CR^{f},
each R^{f} is independently hydrogen, halogen, alkyl, cycloalkyl, alkenyl, cycloalkenyl, alkynyl, aryl, fused aryl-cycloalkyl, fused aryl-heterocycloalkyl, heteroaryl, heteroaryl substituted with at least one aryl group, benzyl, haloalkyl, haloalkenyl, —NH₂, —NHR″, —NR″₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OH, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, —S(O)₂NR″₂, —R²¹, —O—R²¹, —NH—R²¹, —C(O)—NH—R²¹, or —CH₂—NH—C(O)—R²¹; or when Y₁ and Y₂ are CR^{f} then each R^{f}, together with the carbon atom to which it is attached, forms a 5- or 6-membered ring;
each R^{c} is independently hydrogen, halogen, alkyl, cycloalkyl, alkenyl, cycloalkenyl, alkynyl, aryl, aryl substituted with at least one —OR″, heteroaryl, benzyl, haloalkyl, haloalkenyl, —NH₂, —NHR″, —NR″₂, —CH₂NH₂, —NHC(O)R″, —NR″C(O)R″, NHC(O)CH(OH)R″, —NR″C(O)CH(OH)R″, —NHC(O)OR″, —NR″C(O)OR″, —NHSO₂R″, —NR″SO₂R″, —NO₂, —CN, —C(O)H, C(O)R″, —C(O)OR″, —C(O)NH₂, —C(O)NHR″, —C(O)NR″₂, —OH, —OR″, —OC(O)H, —OC(O)R″, —OC(O)OH, —OC(O)OR″, —OC(O)NH₂, —OC(O)NHR″, —OC(O)NR″₂, —SH, —SR″, —S(O)₂H, —S(O)₂R″, —S(O)₂OH, —S(O)₂OR″, —S(O)₂NH₂, —S(O)₂NHR″, —S(O)₂NR″₂, —O—R²¹, —NH—R²¹, —C(O)—NH—R²¹, or —CH₂—NH—C(O)—R²¹;
each R⁴ is independently hydrogen, halogen, alkyl, cycloalkyl, alkenyl, cycloalkenyl, alkynyl, aryl, heteroaryl, benzyl, haloalkyl, haloalkenyl, —C(O)H, C(O)R″, —C(O)OH, —C(O)OR″, —C(O)NH₂, —C(O)NHR″, —C(O)NR″₂, —OH, —OR″, —NH₂, —NHR″, —NR″₂, —S(O)₂H, —S(O)₂R″, —R²¹, —C(O)—NH—R²¹, or —CH₂—NH—C(O)—R²¹; and
each R″ is independently alkyl, cycloalkyl, alkenyl, cycloalkenyl, aryl, heteroaryl, or benzyl;
R²¹ is a bond connected to R¹⁸ of the linker, wherein Formula (III) contains a single R²¹;
wherein, when n=2, each R^{c} is hydrogen, and each of G₁, G₂, G₃ and G₄ is CR^{c}, then C═X₁ may be replaced by CH;
and wherein:
(i) when R^{x} is 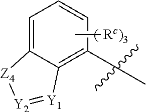
and Z₄ is NH, then L₁ is hydrogen, —CH₂C(O)OR″, or —OR″;
(ii) when R^{x} is 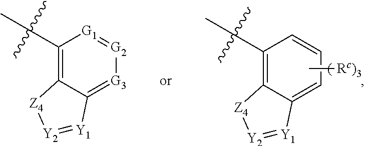
Z₄ is NR⁴, Y₁ is CR^{f}, and Y₂ is N, then R⁴ is not alkyl and at least one of R² and R is not H;
(iii) when R^{x} is 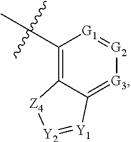
Z₄ is NR⁴, and Y₁ and Y₂ are CR^{f}, then at least one of G₁, G₂ and G₃ is N;
(iv) when Z₄ is NR⁴, and Y₁ and Y₂ are CR^{f}, then R^{x} is not 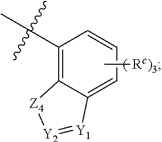
(v) when R^{x} is 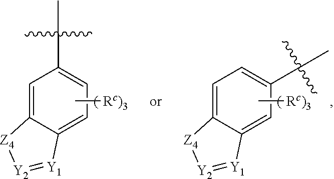
Z₄ is NR⁴, and Y₁ or Y₂ is N, then R⁴ is not alkyl;
(vi) when R^{x} is 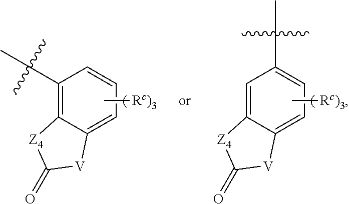
then n=1 or 2; and
(vii) when R^{x} is 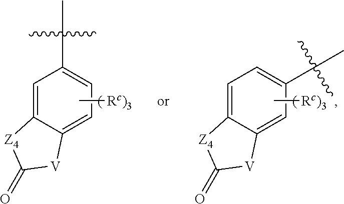
wherein
[MCL-1 ligand moiety] is a compound of Formula (A), Formula (B) or Formula (C) 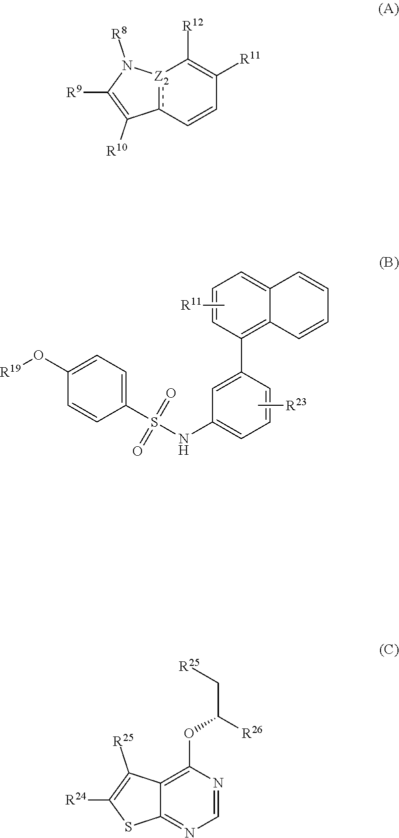
wherein [symbol]
is a single bond or a double bond;
R⁸ is H, R¹⁹, or C₁-C₆ alkyl optionally substituted with morpholine;
R⁹ is —C(O)OH, —C(O)OC₁-C₆alkyl; —C(O)NH₂; —C(O)OR¹⁹ or —C(O)NHR¹⁹,
R¹⁰ is —C₂₋₅alkyl-O—R¹³ or —C₂₋₅alkyl-NMe-R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹¹ is H, halogen or C₁-C₆ alkyl,
R¹² is H, 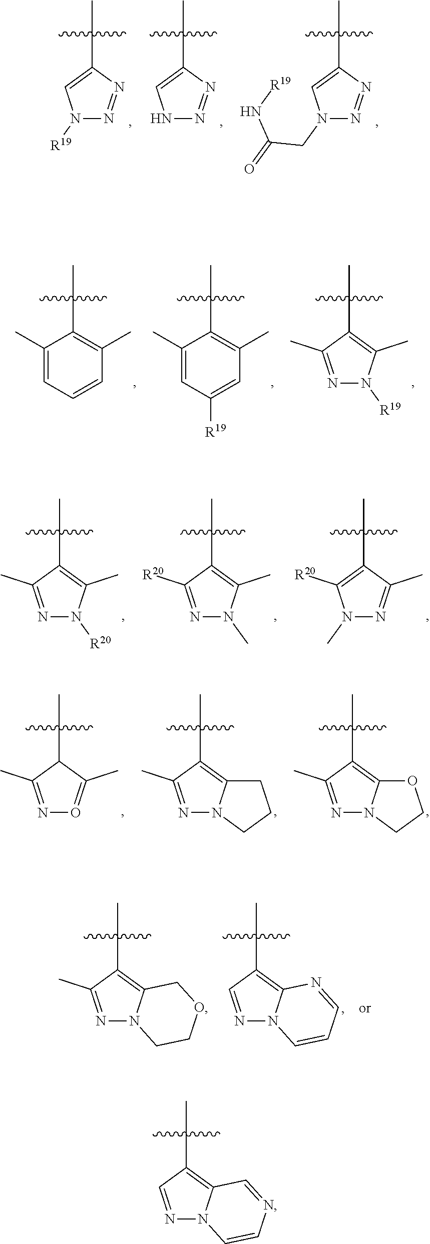
wherein R²⁰ is Me, —CH₂—OMe, —CH₂—O-bromobenzaldehyde, or 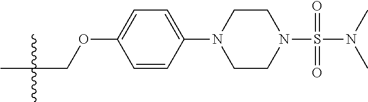 [SMILES: *COc1ccc(N2CCN(S(=O)(=O)N(C)C)CC2)cc1]
or when R¹² is 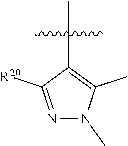
and R¹⁰ is —O-naphthyl substituted with —O— or —S—, then R²⁰ is 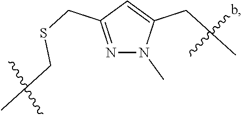
wherein [symbol] indicates attachment to —O— or —S— of R¹⁰;
and wherein
R¹⁹ is a bond connected to R¹⁴ of the linker;
R²³ is —C(O)OH or —C(O)OC₁-C₆alkyl;
Z₂ is N or C, wherein when Z₂ is N, then [symbol] is a single bond; and when Z₂ is C, then [symbol] is a double bond,
R²⁴ is furan optionally substituted with at least one halogen,
each R²⁵ is independently phenyl substituted with —OR²⁸ and optionally further substituted with at least one substituent selected from halogen and C₁-C₆ alkyl;
R²⁶ is —C(O)OR¹⁹ or —C(O)NHR¹⁹; and
each R²⁸ is independently —C₁₋₃alkyl-(N-alkyl piperazine) or —C₁₋₃alkyl-(N-haloalkylpyrazole)
and wherein each of Formula (A), Formula (B) and Formula (C) contains a single R¹⁹;
and wherein [linker] has the following formula
R¹⁴—R¹⁵—R¹⁶—R¹⁷—R¹⁸
wherein
R¹⁴ is —C₁₋₆alkyl, —C₂₋₆alkenyl, —C₂₋₆alkynyl, C₁₋₆ alkyl-N(C₁₋₆ alkyl)-, —C(O)—, —SO₂— or is absent
R¹⁵ is cycloalkyl, heterocycloalkyl, aryl, heteroaryl, —C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, (C₂H₄—O)ₓ, (C₃H₆—O)ₓ, or is absent
x is 1-10
y is 2-10
R¹⁸ is —C₁₋₆ alkyl, heterocycloalkyl, or is absent
wherein at least one of R¹⁴-R¹⁸ is present

105. The compound of claim 104, wherein each alkyl, alkenyl, alkynyl, aryl, heteroaryl and benzyl is unsubstituted.

106. The compound of any one of claims 104-105, wherein in Formula (III):
each of X₁ and X₂ is O;
T is C═O;
R¹ is hydrogen,
L₁ is hydrogen,
R^{x} is 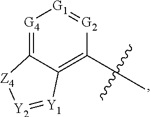
Z₄ is NR⁴;
each of G₁, G₂ and G₄ is CR^{c},
Y₁ is N, and
Y₂ is CR^{f}, wherein R^{f} is not hydrogen.

107. The compound of any one of claims 104-106 wherein [ligase ligand moiety] is Formula (III):

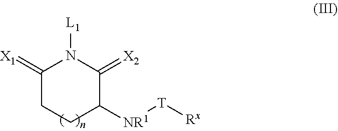

108. The compound of any one of claims 104-107, wherein one of R is —O—R²¹, —NH—R²¹, —C(O)—NH—R²¹, or —CH₂—NH—C(O)—R²¹.

109. The compound of any one of claims 104-108, wherein G₁ is C—O—R²¹, C—NH—R²¹, C—C(O)—NH—R²¹, or C—CH₂—NH—C(O)—R²¹.

110. The compound of any one of claims 104-108, wherein G₂ is C—O—R²¹, C—NH—R²¹, C—C(O)—NH—R²¹, or C—CH₂—NH—C(O)—R²¹.

111. The compound of any one of claims 104-107, wherein R⁴ is R²¹, —C(O)—NH—R²¹, or —CH₂—NH—C(O)—R²¹.

112. The compound of any one of claims 104-107, wherein one of R^{f} is —R²¹, —O—R²¹, —NH—R²¹, —C(O)—NH—R²¹, or —CH₂—NH—C(O)—R²¹.

113. The compound of claim 112, wherein Y₂ is C—R²¹, CO—R²¹, C—NH—R²¹, C—C(O)—NH—R²¹, or C—CH₂—NH—C(O)—R²¹.

114. The compound of any one of claims 104-113, wherein [ligase ligand moiety] is selected from

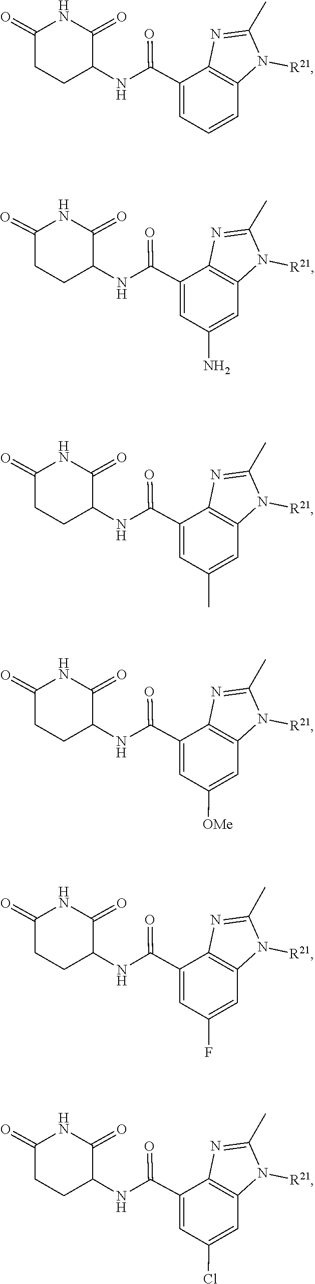

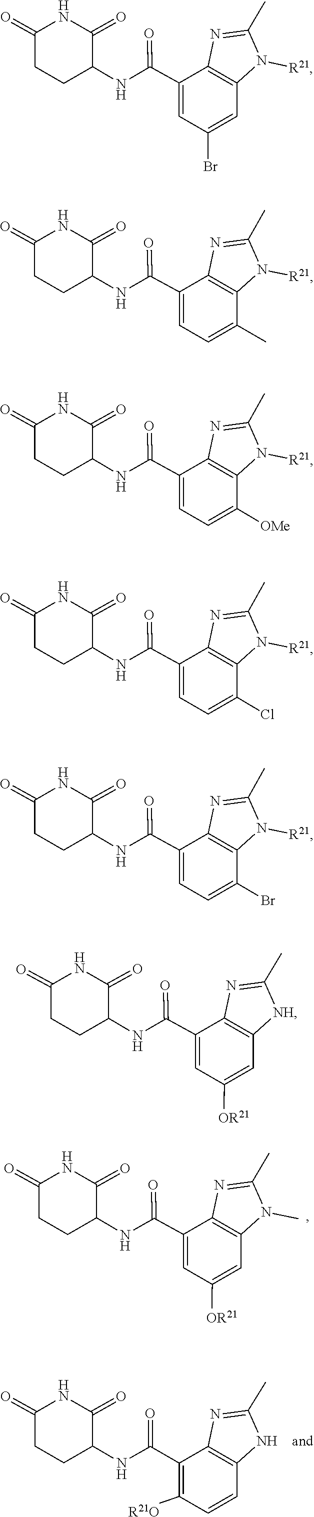

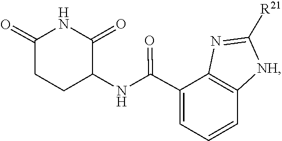

115. The compound of any one of claims 104-105, wherein [ligase ligand moiety] is of Formula (II):

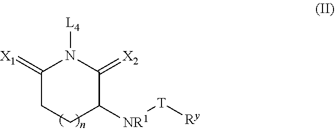

116. The compound of any one of claims 104-105 and 115, wherein R^{y} is selected from

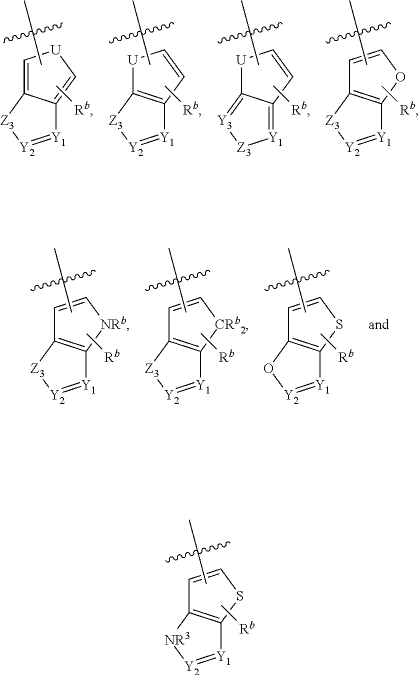

117. The compound of any one of claims 104-105 and 115-116, wherein
Z₃ is S or NR³;
U is O or S;
each of Y₁, Y₂ and Y₃ is independently N or CR^{d}.

118. The compound of any one of claims 104-105 and 115-117, wherein R^{b} is hydrogen or alkyl.

119. The compound of any one of claims 104-105 and 115-118, wherein R³ is hydrogen, alkyl, cycloalkyl, —R²¹, —C(O)—NH—R²¹, or —CH₂—NH—C(O)—R²¹.

120. The compound of any one of claims 104-105 and 115-119, wherein each R^{d} is independently hydrogen, alkyl, —O—R²¹, —NH—R²¹, —C(O)—NH—R²¹, or —CH₂—NH—C(O)—R²¹.

121. The compound of any one of claims 104-120, wherein R¹⁴ is —C₁₋₆ alkyl, —C₂₋₆ alkenyl, —C₂₋₆ alkynyl, —C(O)—, —SO₂— or is absent.

122. The compound of any one of claims 104-121, wherein R¹⁵ is cycloalkyl, heterocycloalkyl, aryl, heteroaryl, —C₁₋₆ alkyl-NH—, -cycloalkyl-NH— or is absent.

123. The compound of any one of claims 104-122, wherein
R¹⁴ is —C₁₋₆ alkyl, —SO₂— or is absent
R¹⁵ is piperazine, bridged piperazine, piperazine N-oxide, piperazine cation, —C₁₋₆ alkyl-NH—, 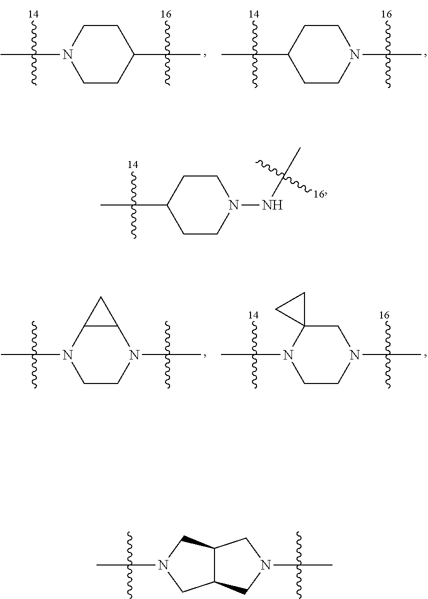 [SMILES: *C1CCN(*)CC1 ; *C1CCN(*)CC1 ; *NN1CCC(*)CC1 ; *N1CCN(*)C2CC21 ; *N1CCN(*)C2(CC2)C1 ; *N1C[C@H]2CN(*)C[C@H]2C1]
or is absent, wherein [symbol] indicates attachment to R¹⁴ and [symbol] indicates attachment to R¹⁶,
R¹⁶ is —C₁₋₆ alkyl, —C(O)—, —C(O)—NH—, —CH₂—C(O)—NH— or is absent
R¹⁷ is —CH₂(C₂H₄—O)_{y}, (C₂H₄—O)ₓ, (C₃H₆—O)ₓ, or is absent
x is 1-6
y is 2-6
R¹⁸ is —C₁₋₆ alkyl, piperazine, or is absent
wherein at least one of R¹⁴-R¹⁸ is present.

124. The compound of any one of claims 104-123, wherein R¹⁸ is —C₁₋₆ alkyl or is absent.

125. The compound of any one of claims 104-124, wherein when R¹⁵ is piperazine, bridged piperazine, piperazine N-oxide, piperazine cation, —C₁₋₆ alkyl-NH—, 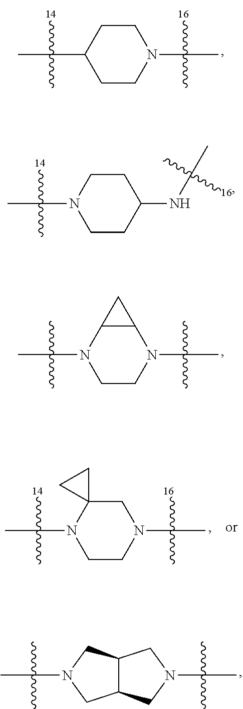 [SMILES: *C1CCN(*)CC1 ; *NC1CCN(*)CC1 ; *N1CCN(*)C2CC21 ; *N1CCN(*)C2(CC2)C1 ; *N1C[C@H]2CN(*)C[C@H]2C1]
then R¹⁴ is —C₁₋₆ alkyl.

126. The compound of any one of claims 104-125, wherein
R¹⁴ is —C₁₋₆ alkyl,
R¹⁵ is piperazine, bridged piperazine, piperazine N-oxide, 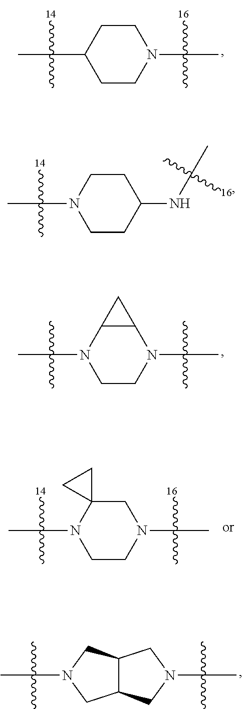 [SMILES: *C1CCN(*)CC1 ; *NC1CCN(*)CC1 ; *N1CCN(*)C2CC21 ; *N1CCN(*)C2(CC2)C1 ; *N1C[C@H]2CN(*)C[C@H]2C1]
R¹⁶ is —C(O)—, —CH₂—C(O)—NH—, or is absent
R¹⁷ is —CH₂(C₂H₄—O)_{y}, (C₂H₄—O)ₓ, (C₃H₆—O)ₓ, or is absent
R¹⁸ is —C₁₋₆ alkyl.
wherein when R¹⁶ and R¹⁷ are absent, R¹⁸ is —C₃₋₆ alkyl.

127. The compound of claim 126, wherein
R¹⁴ is —C₂ alkyl,
x is 1, 2 or 6
y is 2.

128. The compound of claim 126, wherein
R¹⁵ is piperazine,
R¹⁶ is —C(O)—,
R¹⁷ is - absent.

129. The compound of claim 128, wherein
R¹⁴ is —C₂ alkyl,
R¹⁸ is —C₁₋₂ alkyl.

130. The compound of any one of claims 104-125, wherein when R¹⁴ is —SO₂—, at least two of R¹⁵-R¹⁸ are present, and at least one of R¹⁵—R¹⁸ is not C₁₋₆ alkyl.

131. The compound of any one of claims 104-125, wherein
R¹⁴ is —SO₂—
R¹⁵ is —C₁₋₆ alkyl-NH—
R¹⁶ is —C(O)—
R¹⁷ is —CH₂(C₂H₄—O)_{y}, (C₂H₄—O)ₓ or is absent
R¹⁸ is —C₂₋₄ alkyl.

132. The compound of claim 131, wherein
R¹⁵ is —C₂alkyl-NH—
x is 1 or 2
y is 1
R¹⁸ is —C₂₋₄ alkyl

133. The compound of any one of claims 104-125, wherein
R¹⁴ is absent
R¹⁵ is absent
R¹⁶ is —C(O)—NH—, or is absent
R¹⁷ is —CH₂(C₂H₄—O)_{y}, (C₂H₄—O)ₓ, (C₃H₆—O)ₓ, or is absent
R¹⁸ is —C₁₋₆ alkyl.

134. The compound of any one of claims 104-133, wherein at least one of R¹⁴-R¹⁸ is not —C₁₋₆ alkyl.

135. The compound of claim 133 or 134, wherein
x is 1, 2 or 3
y is 2
R¹⁸ is —C₂₋₆ alkyl.

136. The compound of any one of claims 104-135, wherein when R¹⁵ is —C₁₋₆ alkyl-NH—, at least one of R¹⁶—R¹⁸ is present.

137. The compound of any one of claims 104-136 wherein when R¹⁷ is —CH₂(C₂H₄—O)_{y}, (C₂H₄—O)ₓ or (C₃H₆—O)ₓ, at least one of R¹⁴—R¹⁶ and R¹⁸ is present, wherein at least one of R¹⁴ and R¹⁸ is not —C₁₋₆ alkyl.

138. The compound of any one of claims 104-137, wherein [linker] is selected from 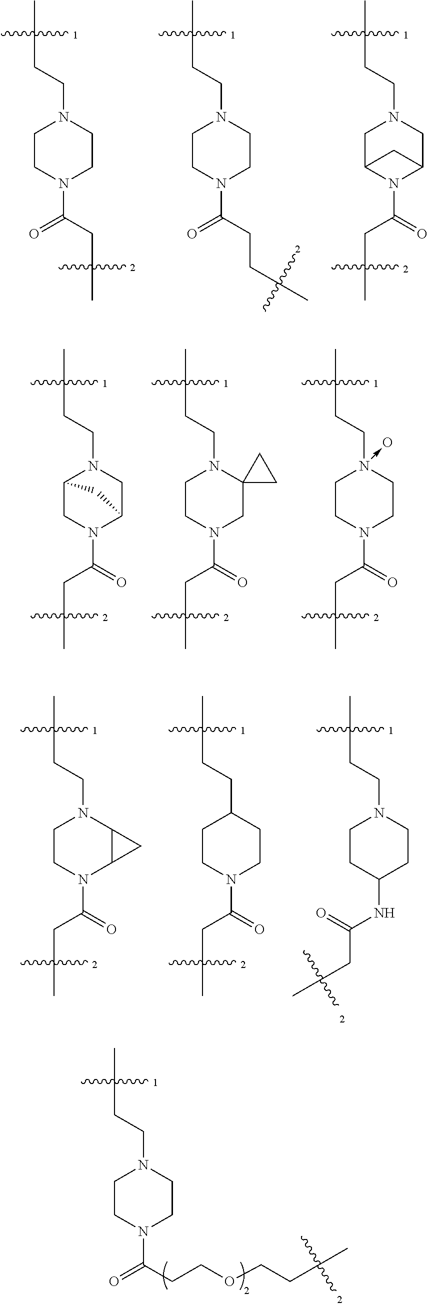 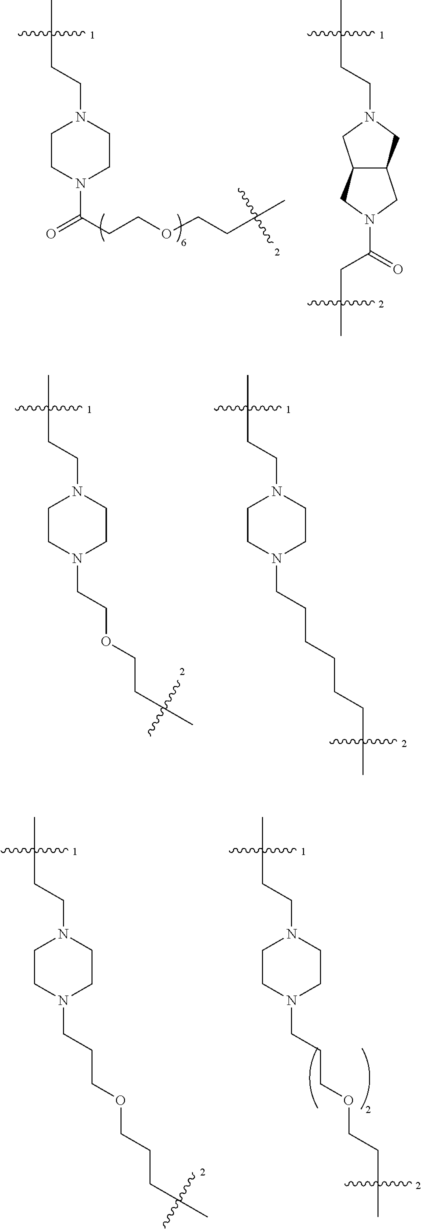 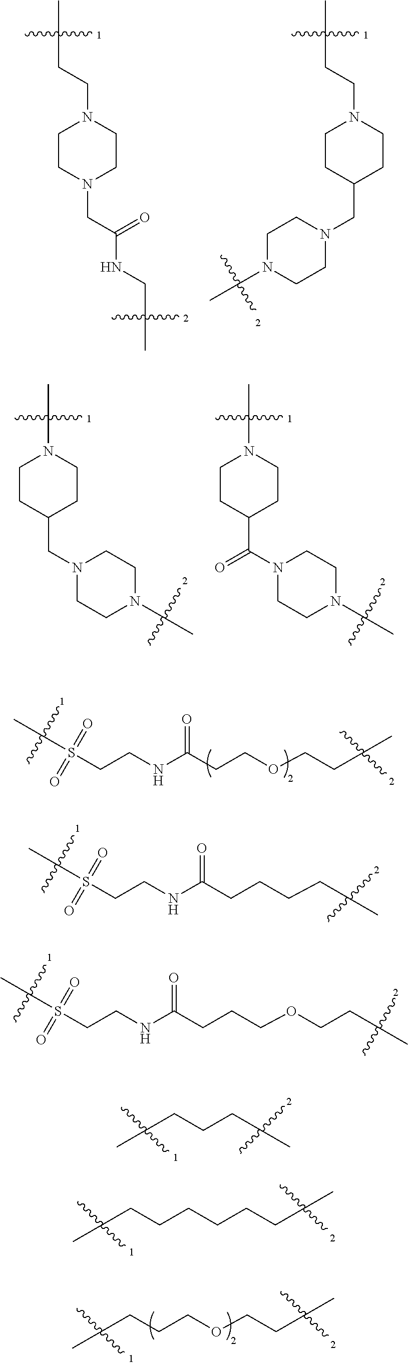 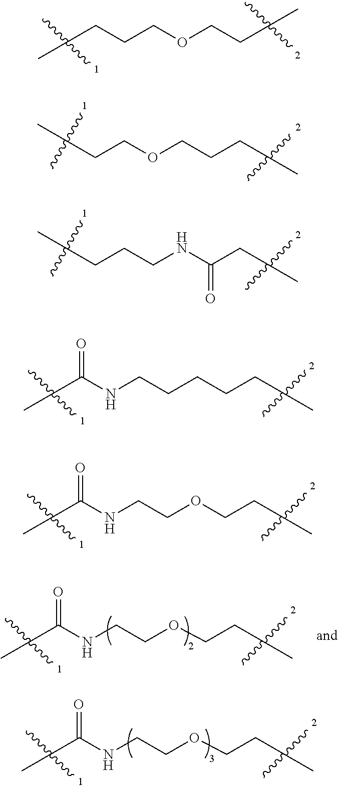
wherein [symbol]
indicates attachment to [MCL-1 ligand moiety] and [symbol]
indicates attachment to [ligase ligand moiety].

139. The compound of any one of claims 104-138, wherein [linker] is selected from
wherein 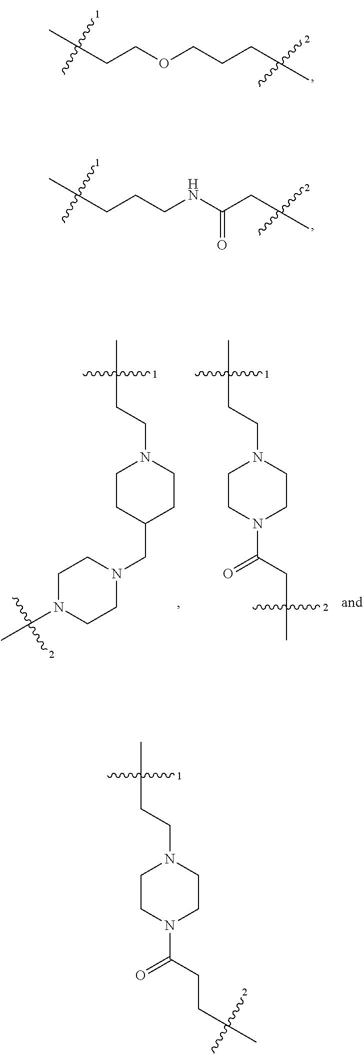 [SMILES: *CCCOCC* ; *CCCNC(=O)C* ; *CCN1CCC(CN2CCN(*)CC2)CC1 ; *CCN1CCN(C(=O)C*)CC1 ; *CCC(=O)N1CCN(CC*)CC1] [symbol]
indicates attachment to [MCL-1 ligand moiety] and [symbol]
indicates attachment to [ligase ligand moiety].

140. The compound of any one of claims 104-139, wherein R¹⁰ is —C₂₋₅alkyl-O—R¹³ wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the naphthyl is optionally substituted with —O— or —S—.

141. The compound of any one of claims 104-140, wherein R¹² is H,

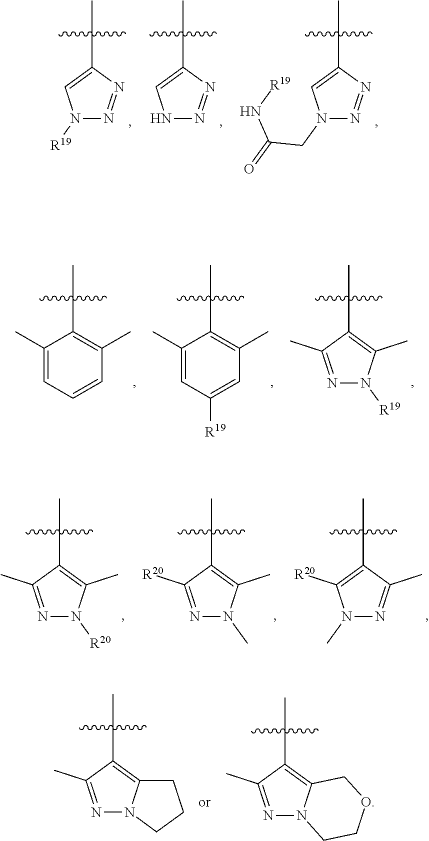

142. The compound of any one of claims 104-141, wherein R²⁰ is Me, —CH₂—O-bromobenzaldehyde, or

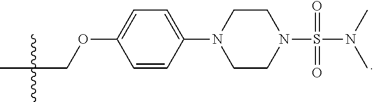 [SMILES: *COc1ccc(N2CCN(S(=O)(=O)N(C)C)CC2)cc1]

143. The compound of any one of claims 104-142 wherein when R⁸ is H, R¹³ is

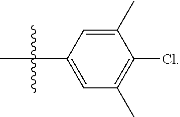 [SMILES: *c1cc(C)c(Cl)c(C)c1]

144. The compound of any one of claims 104-143, wherein
R⁸ is H, R¹⁹, methyl, or —CH₂CH₂-morpholine;
R⁹ is —C(O)OH or —C(O)NHR¹⁹,
R¹⁰ is —C₃H₆O—R¹³,
wherein R¹³ is 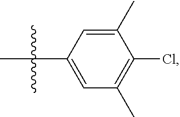 [SMILES: *c1cc(C)c(Cl)c(C)c1]
tetraline or naphthyl optionally substituted with fluorine;
R¹¹ is H, Cl, F or methyl,
R¹² is 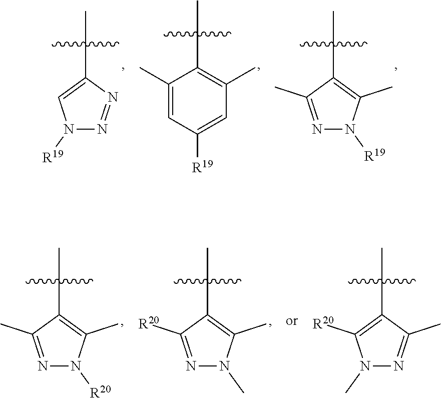
wherein R²⁰ is Me, —CH₂—O-bromobenzaldehyde, or

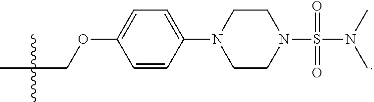 [SMILES: *COc1ccc(N2CCN(S(=O)(=O)N(C)C)CC2)cc1]

145. The compound of claim 144, wherein
R⁸ is R¹⁹ or methyl;
R¹⁰ is —C₃H₆O—R³, wherein R¹³ is naphthyl optionally substituted with fluorine;
R¹¹ is Cl or F,
R¹² is

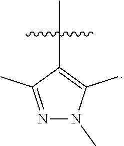

146. The compound of any one of claims 104-145 wherein Z₂ is C and [symbol] is a double bond.

147. The compound of any one of claims 104-146, wherein [MCL-1 ligand moiety] is

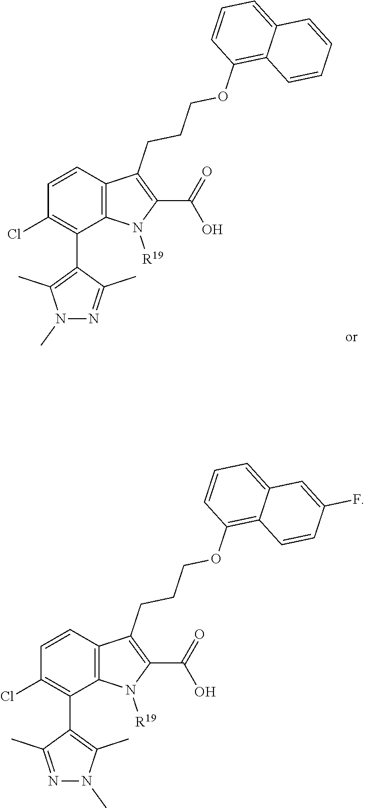

148. The compound of claim 147, wherein the compound is selected from

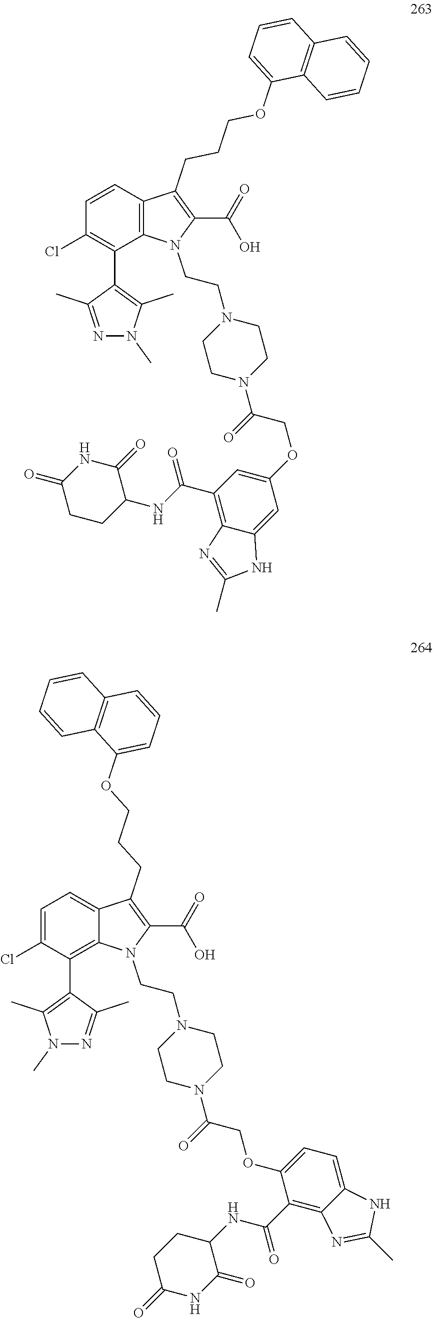

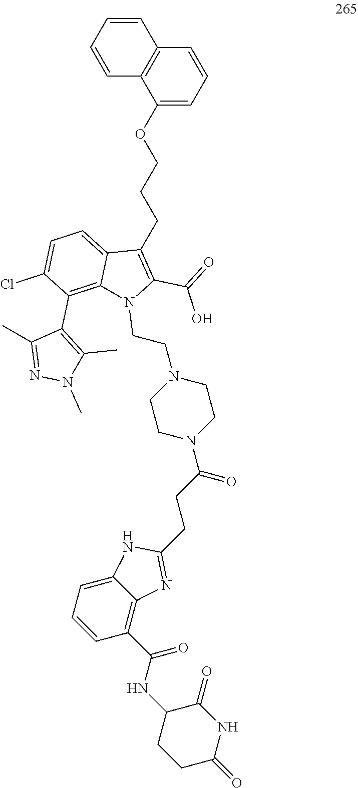 [SMILES: Cc1nn(C)c(C)c1-c1c(Cl)ccc2c(CCCOc3cccc4ccccc34)c(C(=O)O)n(CCN3CCN(C(=O)CCc4nc5c(C(=O)NC6CCC(=O)NC6=O)cccc5[nH]4)CC3)c12]

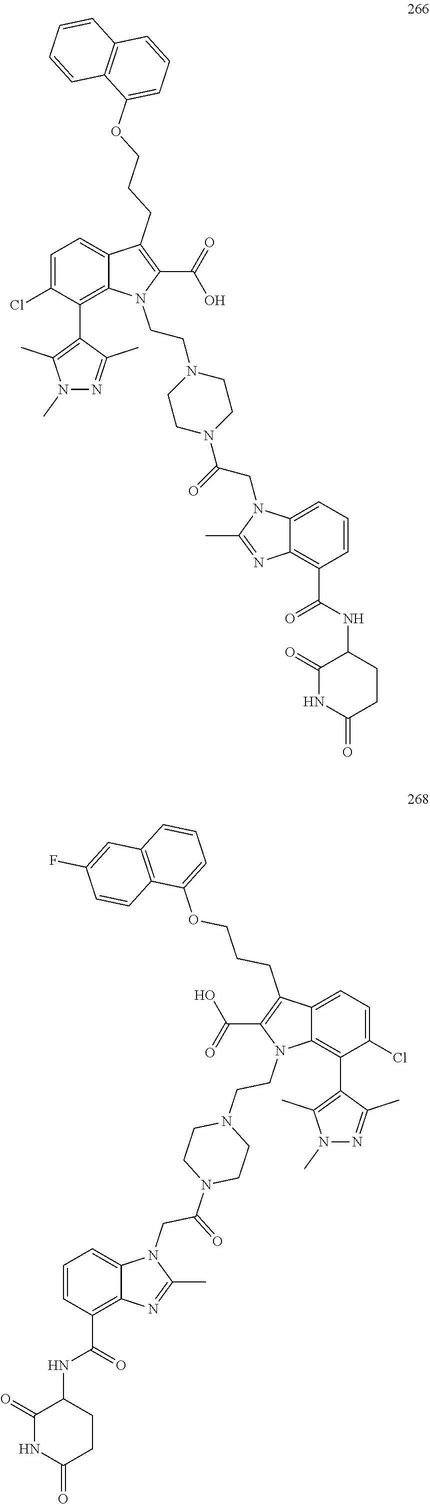 [SMILES: Cc1nn(C)c(C)c1-c1c(Cl)ccc2c(CCCOc3cccc4ccccc34)c(C(=O)O)n(CCN3CCN(C(=O)Cn4c(C)nc5c(C(=O)NC6CCC(=O)NC6=O)cccc54)CC3)c12 ; Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cccc54)CC3)c12]

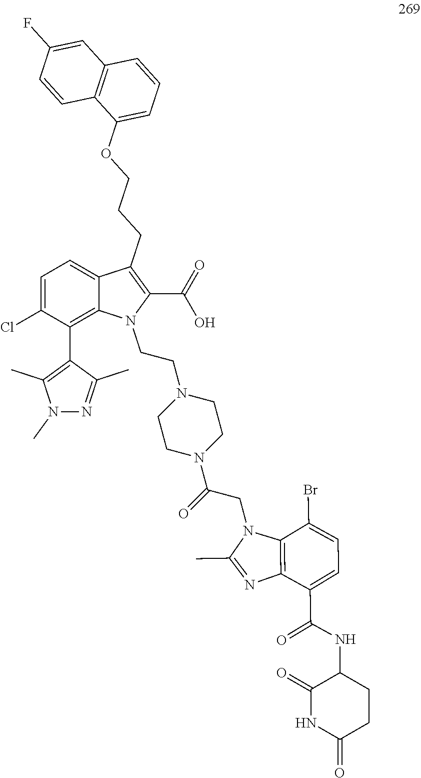 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)ccc(Br)c54)CC3)c12]

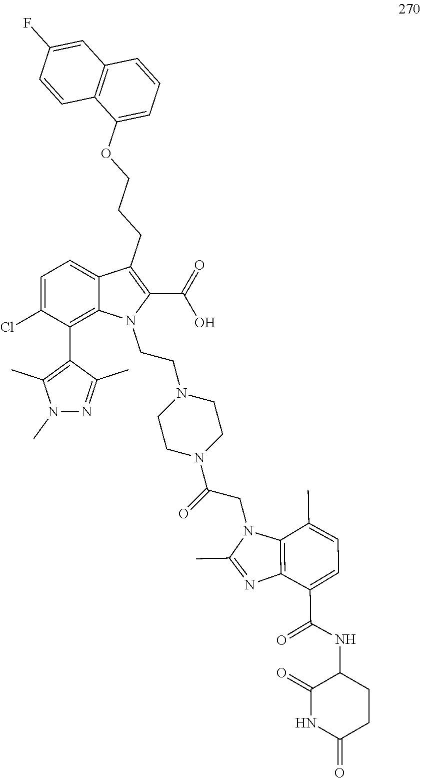 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)ccc(C)c54)CC3)c12]

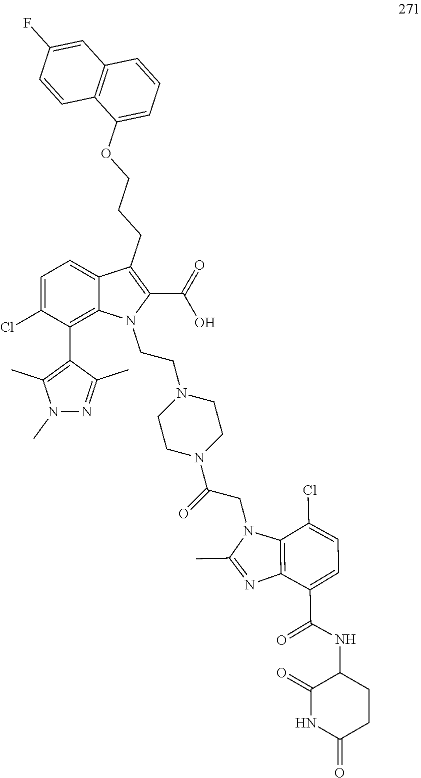 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)ccc(Cl)c54)CC3)c12]

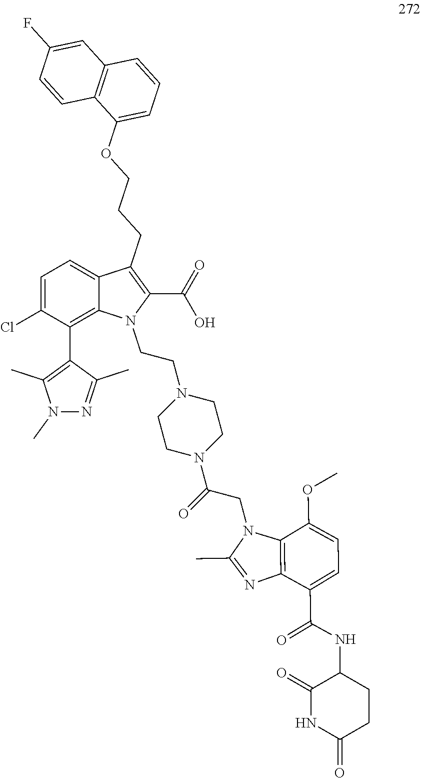 [SMILES: COc1ccc(C(=O)NC2CCC(=O)NC2=O)c2nc(C)n(CC(=O)N3CCN(CCn4c(C(=O)O)c(CCCOc5cccc6cc(F)ccc56)c5ccc(Cl)c(-c6c(C)nn(C)c6C)c54)CC3)c12]

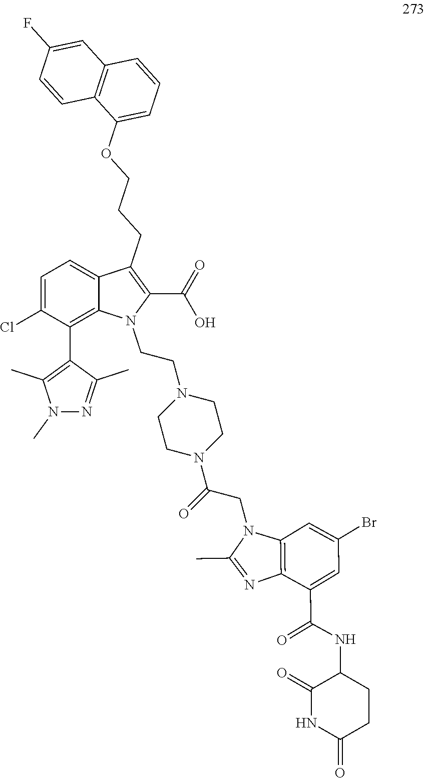 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cc(Br)cc54)CC3)c12]

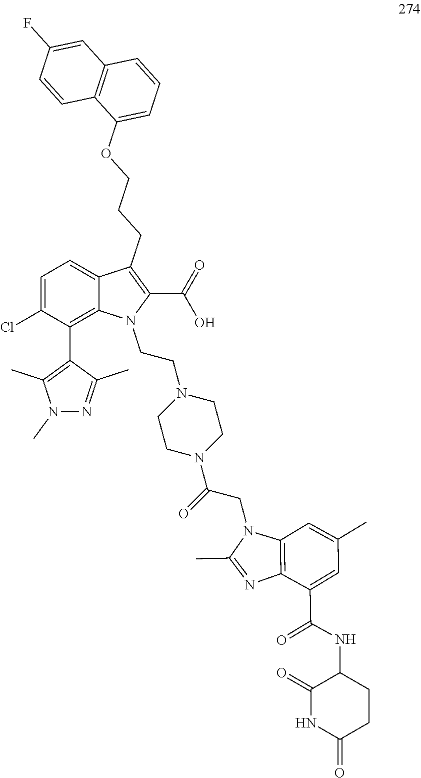 [SMILES: Cc1cc(C(=O)NC2CCC(=O)NC2=O)c2nc(C)n(CC(=O)N3CCN(CCn4c(C(=O)O)c(CCCOc5cccc6cc(F)ccc56)c5ccc(Cl)c(-c6c(C)nn(C)c6C)c54)CC3)c2c1]

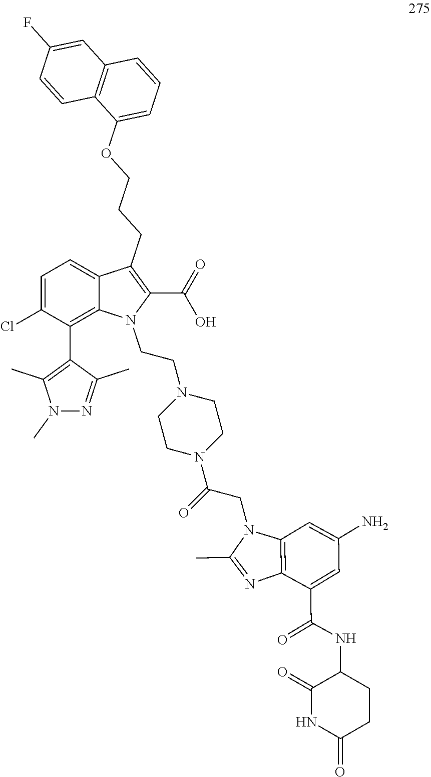 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cc(N)cc54)CC3)c12]

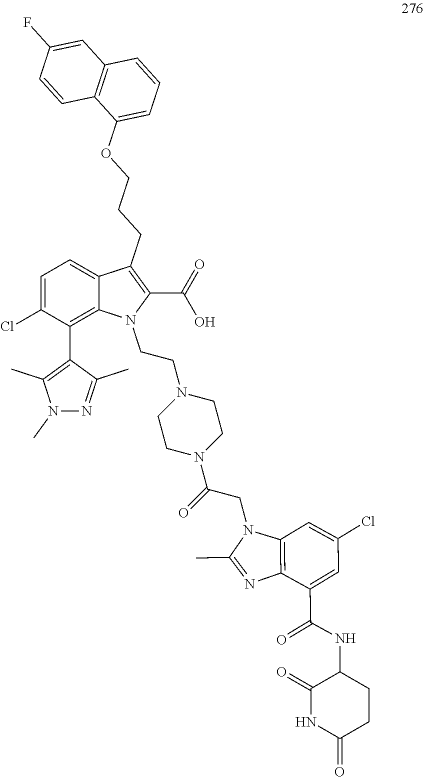 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cc(Cl)cc54)CC3)c12]

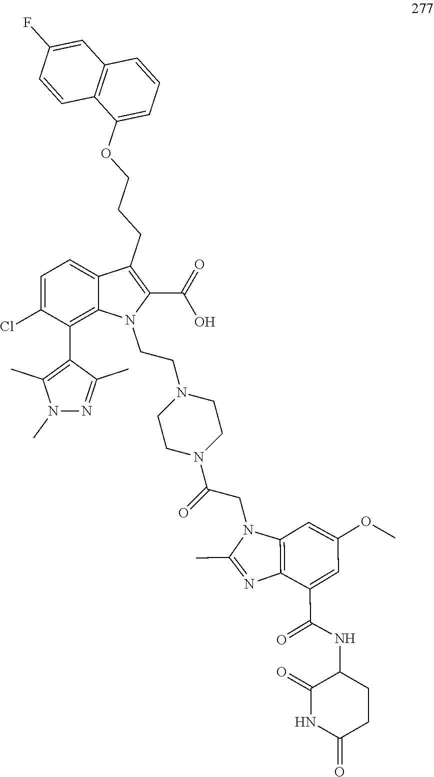

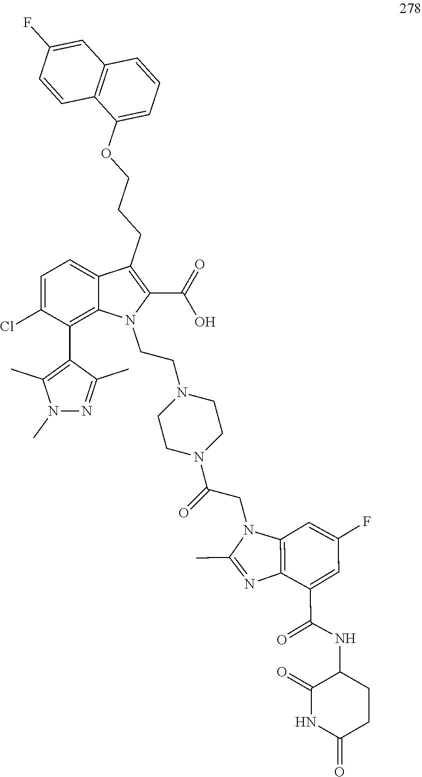 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cc(F)cc54)CC3)c12]

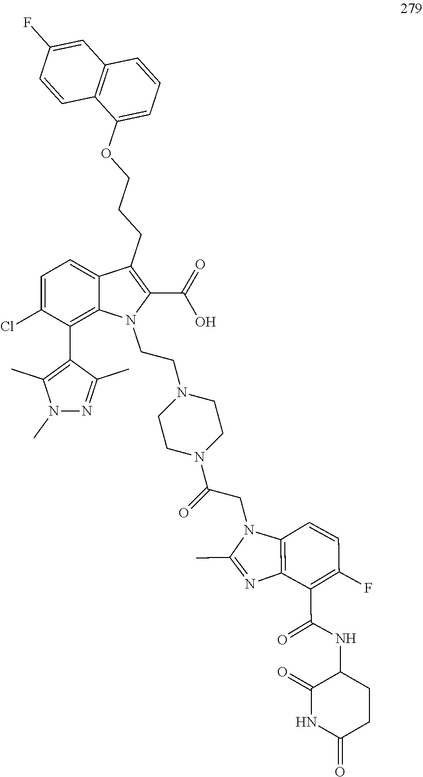 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)c(F)ccc54)CC3)c12]

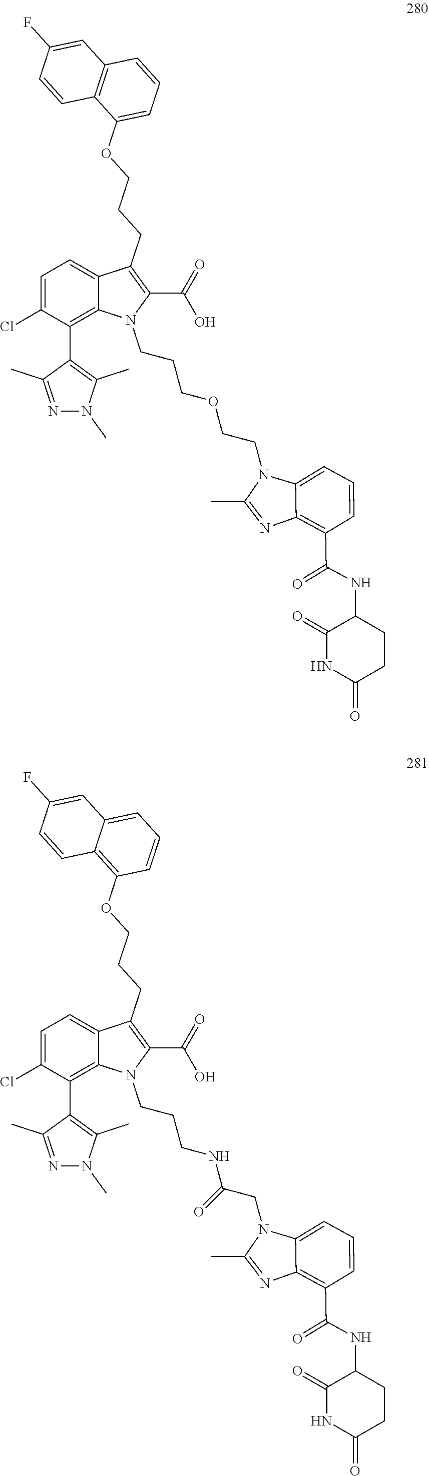 [SMILES: Cc1nn(C)c(C)c1-c1c(Cl)ccc2c(CCCOc3cccc4cc(F)ccc34)c(C(=O)O)n(CCCOCCn3c(C)nc4c(C(=O)NC5CCC(=O)NC5=O)cccc43)c12 ; Cc1nn(C)c(C)c1-c1c(Cl)ccc2c(CCCOc3cccc4cc(F)ccc34)c(C(=O)O)n(CCCNC(=O)Cn3c(C)nc4c(C(=O)NC5CCC(=O)NC5=O)cccc43)c12]

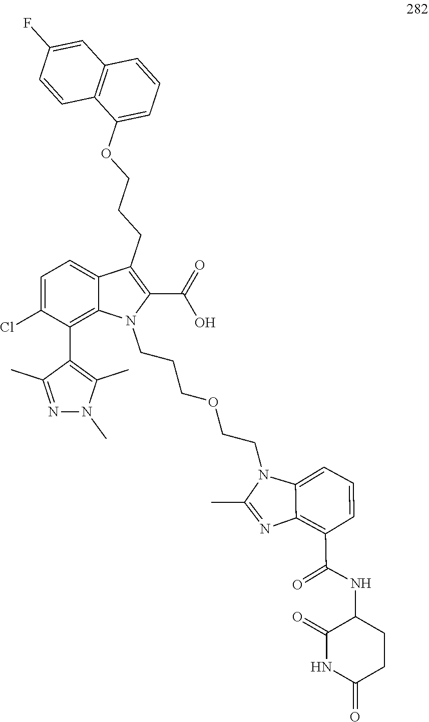 [SMILES: Cc1nn(C)c(C)c1-c1c(Cl)ccc2c(CCCOc3cccc4cc(F)ccc34)c(C(=O)O)n(CCCOCCn3c(C)nc4c(C(=O)NC5CCC(=O)NC5=O)cccc43)c12]

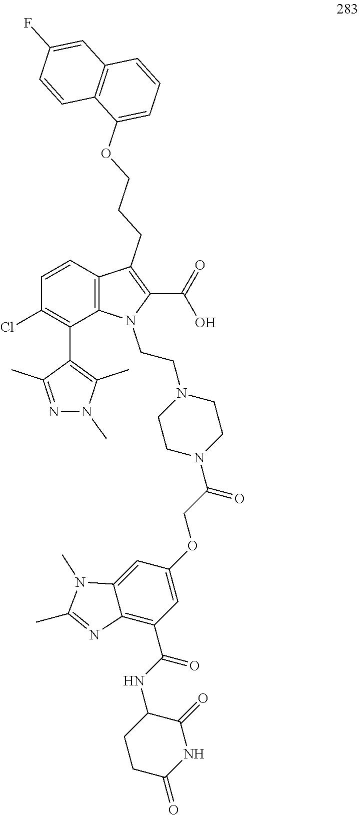 [SMILES: Cc1nn(C)c(C)c1-c1c(Cl)ccc2c(CCCOc3cccc4cc(F)ccc34)c(C(=O)O)n(CCN3CCN(C(=O)COc4cc(C(=O)NC5CCC(=O)NC5=O)c5nc(C)n(C)c5c4)CC3)c12]

149. The compound of claim 189, wherein the compound is selected from:

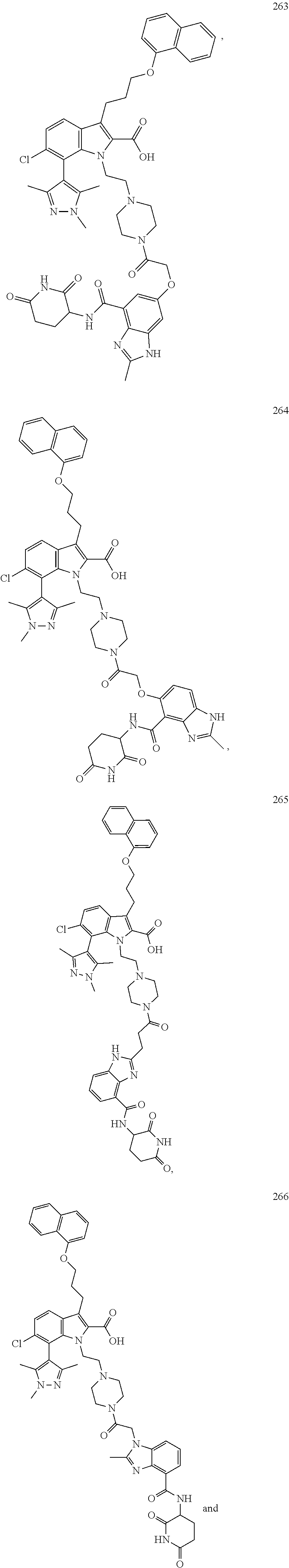

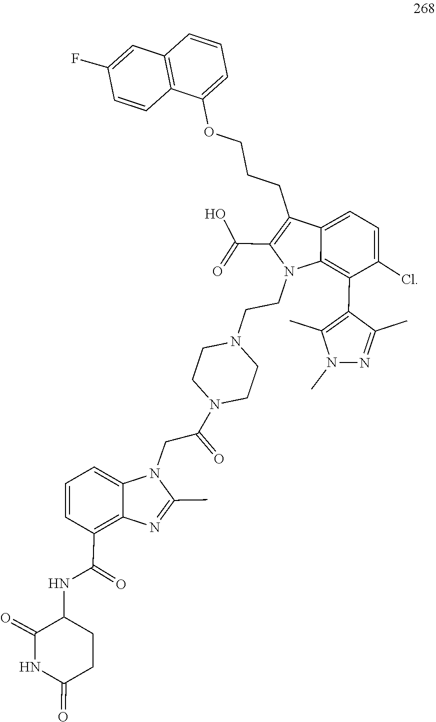 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cccc54)CC3)c12]

150. The compound of any preceding claim, wherein T is C═O.

151. The compound of any one of claims 1-149, wherein T is SO₂.

152. The compound of any preceding claim, wherein X₁ and X₂ are O.

153. The compound of any one of claims 1-151, wherein X₁ is O and X₂ is S.

154. The compound of any one of claims 1-151, wherein X₁ is S and X₂ is O.

155. The compound of any one of claims 1-151, wherein X₁ and X₂ are S.

156. The compound of any preceding claim, wherein n is 0.

157. The compound of any one of claims 1-155, wherein n is 1 or 2.

158. The compound of claim 157, wherein n is 1.

159. The compound of claim 157, wherein n is 2.

160. The compound of any preceding claim, wherein [MCL-1 ligand moiety] is a compound of Formula (A), and wherein R¹⁰ is —C₂₋₅alkyl-O—R¹³.

161. The compound of any preceding claim, wherein R¹⁰ is —C₃H₆—O—R¹³.

162. A pharmaceutical composition comprising a compound of any one of claims 1-161.

163. The compound of any one of claims 1-161 or the pharmaceutical composition of claim 162, for use in medicine.

164. The compound of any one of claims 1-161 or the pharmaceutical composition of claim 162, for use in the treatment of cancer.

165. The compound or composition for use of claim 164, wherein the cancer is selected from breast cancer, triple negative breast cancer, colorectal cancer, pancreatic cancer, skin cancer, melanoma, ovarian cancer, kidney cancer, lung cancer, small-cell lung cancer, non-small-cell lung cancer, lymphoma, non-Hodgkin's lymphoma, multiple myeloma, cervical cancer, leukaemia, chronic lymphocytic leukaemia (CLL), acute myeloid leukaemia (AML), chronic myelogenous leukaemia (CML), acute lymphoblastic leukaemia (ALL), bladder cancer, and prostate cancer.

166. The compound or composition for use of claim 165, wherein the cancer is multiple myeloma or acute myeloid leukaemia.

167. A method of treating cancer in a subject in need thereof, the method comprising administering to the subject an effective amount of a compound according to any one of claims 1-161, or a pharmaceutical composition according to claim 162.

168. The method of claim 167, wherein the cancer is selected from breast cancer, triple negative breast cancer, colorectal cancer, pancreatic cancer, skin cancer, melanoma, ovarian cancer, kidney cancer, lung cancer, small-cell lung cancer, non-small-cell lung cancer, lymphoma, non-Hodgkin's lymphoma, multiple myeloma, cervical cancer, leukaemia, chronic lymphocytic leukaemia (CLL), acute myeloid leukaemia (AML), chronic myelogenous leukaemia (CML), acute lymphoblastic leukaemia (ALL), bladder cancer, and prostate cancer.

169. The method of claim 168, wherein the cancer is multiple myeloma acute myeloid leukaemia.

170. The method of any one of claims 167-169, wherein the administration does not result in cytotoxicity in cardiomyocytes in the subject.

171. The method of any one of claims 167-170, further comprising administering at least one additional active agent to the subject.

172. The method of claim 171, wherein the at least one additional active agent is an anti-cancer agent selected from eribulin; fulvestrant; midostaurin; an immune checkpoint inhibitor selected from anti-pd-1 antibody, anti-pd-11 antibody, and anti pd-1/pd-11 interaction inhibitor; nivolumab; pembrolizumab; atezolizumab; pidilizumab; carfilzomib; venetoclax; cytarabine; anthracyclines; a taxane compound; and hypomethylating agents.

173. The compound of any one of claims 1-161 or the pharmaceutical composition of claim 162, for use in reversing resistance to chemotherapy or targeted cancer therapies.

174. A method of reversing resistance to chemotherapy or targeted cancer therapies in a subject in need thereof, the method comprising administering to the subject an effective amount of a compound according to any one of claims 1-161, or a pharmaceutical composition according to claim 162.

175. A combined preparation of a compound of any one of claims 1-161 and at least one additional active agent, for simultaneous, separate or sequential use in therapy.

176. The combined preparation of claim 175, wherein the at least one additional active agent is an anti-cancer agent selected from eribulin; fulvestrant; midostaurin; an immune checkpoint inhibitor selected from anti-pd-1 antibody, anti-pd-11 antibody, and anti pd-1/pd-11 interaction inhibitor; nivolumab; pembrolizumab; atezolizumab; pidilizumab; carfilzomib; venetoclax; cytarabine; anthracyclines; a taxane compound; and hypomethylating agents.

177. The combined preparation of any one of claims 175-176 wherein the therapy is the treatment of cancer.

178. A compound of formula (X):
[MCL-1 inhibitor]-L-[cereblon binding moiety] (X)
wherein L is a bond or a linker compound.

179. A method of reducing the cardiac cytotoxicity of an MCL-1 inhibitor, comprising coupling a cereblon binding moiety to the MCL-1 inhibitor.

180. The compound of claim 178 or the method of claim 179, wherein the cereblon binding moiety is a [ligase ligand moiety] as defined in any one of claims 1-159.

181. The compound or method of any one of claims 178-180, wherein the MCL-1 inhibitor is an [MCL-1 ligand moiety] as defined in any one of claims 1-159.

182. The compound or method of any one of claims 178-181, wherein the cereblon binding moiety is coupled to the MCL-1 inhibitor by a linker compound, wherein the linker compound is covalently attached to the cereblon binding moiety and the MCL-1 inhibitor.

183. The compound or method of any one of claims 178-182, wherein the linker compound is a [linker] as defined in any one of claims 1-159.
